(19) United States
(12) Patent Application Publication
(10) Pub. No.: US 2024/0150293 A1
(43) Pub. Date: May 9, 2024

(54) SPIROCYCLOHEXANE DERIVATIVES, PHARMACEUTICAL COMPOSITIONS CONTAINING THEM AND THEIR USES AS ANTI-APOPTOTIC INHIBITORS

(71) Applicants: LES LABORATOIRES SERVIER, SURESNES CEDEX (FR); VERNALIS (R&D) LTD, Cambridge (GB)

(72) Inventors: Stephen STOKES, Duns (GB); Simon BEDFORD, Harlow (GB); l-Jen CHEN, Cambridge (GB); James Edward Paul DAVIDSON, Great Shelford (GB); Nicholas DAVIES, Cambridge (GB); Christopher John GRAHAM, Newmarket (GB); Sean Martin MCKENNA, Duxford (GB); Johannes W.G. MEISSNER, Groningen (NL); James Brooke MURRAY, Linton (GB); Rachel Jane PARSONS, Royston Hertfordshire (GB); Stuart RAY, Cottenham (GB); Emma SANDERS, London (GB); Claire Louise WALMSLEY, Ely (GB); Paul Andrew BROUGH, Haverhill Suffolk (GB); András KOTSCHY, Törökbálint (HU); Ágnes PROSZENYÁK, Budapest (HU); Ádám SINAI, Alsózsolca (HU); Balázs Károly BÁLINT, Budapest (HU); Márton CSÉKEI, Dunakeszi (HU); Márton ZWILLINGER, Budapest (HU); Rita GARAMVÖLGYI, Budapest (HU); Szabolcs SIPOS, Budapest (HU); Vilibald KUN, Tárnok (HU); Zoltán SZABÓ, Budapest (HU); Maïa CHANRION, Tournefeuille (FR); Francesca ROCCHETTI, Vienna (AT); Frédéric COLLAND, Puiseux-en-France (FR); Ana Leticia MARAGNO, Saint Germain en Laye (FR); Laura BRESSON, Montrouge (FR)

(21) Appl. No.: 18/262,231

(22) PCT Filed: Jan. 11, 2022
(86) PCT No.: PCT/EP2022/050460

§ 371 Date: Jul. 20, 2023

(30) Foreign Application Priority Data
Jan. 12, 2021 (EP) 21305024.8

(51) Int. Cl. C07D 215/233; A61K 45/06; A61P 35/00; C07C 229/50; C07D 317/70; C07D 401/12; C07D 405/10; C07D 491/056

(52) U.S. Cl. CPC C07D 215/233; A61K 45/06; A61P 35/00; C07C 229/50; C07D 317/70; C07D 401/12; C07D 405/10; C07D 491/056

(57) ABSTRACT

Compounds of Formula (I):

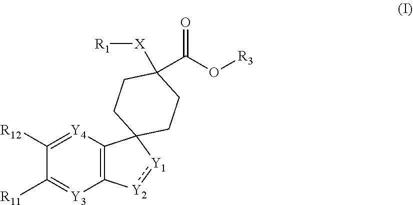

wherein R₁, R₃, R₁₁, R₁₂, X, Y₁, Y₂, Y₃, Y₄ and [symbol] are as defined in the description. Medicinal products containing the same which are useful in treating conditions requiring anti-apoptotic inhibitors.

### FIELD OF THE INVENTION

The present invention relates to new spirocyclohexane derivatives, to processes for their preparation, to pharmaceutical compositions containing them and to their uses as anti-apoptotic inhibitors. The compounds of the present invention inhibit the activity of the Mcl-1 protein and may be of interest in the treatment of cancer, immune and autoimmune diseases.

### BACKGROUND OF THE INVENTION

Apoptosis, or programmed cell death, is a physiological process that is crucial for embryonic development and maintenance of tissue homeostasis.

Apoptotic-type cell death involves morphological changes such as condensation of the nucleus and DNA fragmentation, but also biochemical phenomena such as caspases activation, which causes damage to key structural components of the cell, thus inducing its disassembly and death.

Regulation of apoptosis process is complex and involves the activation or repression of several intracellular signaling pathways (Singh et al, Nature Rev. Mol. Cell. Biol. 2019, 20, 175-193).

Apoptosis deregulation is involved in several pathologies. Increased apoptosis is associated with neurodegenerative disorders such as Parkinson's disease, Alzheimer's disease and ischemia. Conversely, deficits in apoptosis implementation play a significant role in the development of cancers and their chemoresistance, in auto-immune diseases, inflammatory diseases and viral infections. Accordingly, absence of apoptosis is one of the hallmarks of cancer (Hanahan and Weinberg, Cell 2011, 5, 646-674).

The anti-apoptotic proteins of the Bcl-2 family are associated with numerous pathologies. The involvement of proteins of the Bcl-2 family is described in numerous types of cancer, such a colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, pancreatic cancer etc. Overexpression of apoptotic proteins of the Bcl-2 family is involved in tumorigenesis, in resistance to chemotherapy and in the poorer clinical prognosis of patients affected by cancer. Notably, the gene encoding Mcl-1, an anti-apoptotic Bcl-2 family member, is located in one of the most frequently amplified chromosome regions in cancer (Beroukhim et al, Nature 2010, 463, 899-905; Zack et al, Nature Genetics 2013, 45, 1134-1140). In addition, an increasing body of evidences indicates that Mcl-1 is highly expressed in multiple cancer subtypes, including hematological malignancies (reviewed in Wei et al, Blood Rev. 2020, 44, 100672), melanoma (Sale et al, Nat. Commun. 2019, 10, 5167), hepatocellular carcinoma (Sieghart et al, J. Hepatol. 2006, 44, 151-157), breast cancer (Campbell et al, Cell Death Dis. 2018, 9, 19), pancreatic cancer (Castillo et al, Oncogene 2019, 39, 1821-1829), small-cell lung cancer (Yasuda et al, Cell Death Dis. 2020, 11, 177), non-small-cell lung cancer (Wen et al, Diagn. Pathol. 2019, 14, 108), prostate cancer (Reiner et al, Oncoscience 2015, 8, 703-715), urothelial carcinoma (Hong et al, Mol. Cancer Res. 2019, 17, 1294-1304), testicular germ cell tumors (Sano et al, Histopathology 2005, 46, 532-539), etc. In addition, upregulation of Mcl-1 has been implicated in inappropriate survival of virally or bacterially infected cells and in inflammatory conditions, suggesting that interfering with Mcl-1 might be therapeutically beneficial in many other disease settings such as in the diseases of the immune system and autoimmune diseases (Michels et al, Int. J. Biochem. Cell. Biol. 2005, 37, 267-271; Carrington et al, Immunol. Cell Biol. 2017, 95, 870-877; Cottier et al, Rheumatology 2014, 53, 1539-1546).

These findings indicated above motivated the discovery and development of a new class of drugs named BH3 mimetics. These molecules are able to disrupt the interaction between the pro-apoptotic and anti-apoptotic members of the Bcl-2 family and are potent inducers of apoptosis. Particularly, selective inhibitors of Mcl-1, such as A-1210477, S63845, S64315, AMG-176 or AZD-5991, have been discovered (Leverson et al, Cell Death Dis. 2015, 6, e1590; Kotschy et al, Nature 2016, 538, 477-482; Maragno et al, AACR 2019, Poster #4482; Kotschy et al, WO 2015/097123; Caenepeel et al, Cancer Discov. 2018, 8, 1582-1597; Tron et al, Nat. Commun. 2018, 9, 5341) and have shown promising in vivo activity in several types of hematological cell malignancies in preclinical models and three of them—S64315, AMG176 and AZD5991—are currently being investigated in clinical trials (Yang et al, Eur. J. Med. Chem. 2019, 177, 63-75). Consequently, BH3 mimetics represent a highly attractive approach for the development of novel therapies in oncology and in the field of immune and autoimmune diseases. There is, therefore, a high therapeutic need for compounds inhibiting the anti-apoptotic activity of the proteins of the Bcl-2 family and, particularly, there is a high therapeutic need for compounds inhibiting the anti-apoptotic activity of Mcl-1.

### SUMMARY OF THE INVENTION

The present invention provides potent selective Mcl-1 inhibitors of Formula (I) as defined below. We have shown that compounds of Formula (I) have a strong binding affinity on Mcl-1 receptor and are cytotoxic. Moreover, compounds of Formula (I) can induce apoptosis in in vivo cancer models, triggering tumor regression in mice. Based on their ability to induce the apoptosis, the compounds of the invention could be of interest for the treatment of pathologies involving a deregulation in apoptosis, such as, for example, cancer, auto-immune diseases and diseases of the immune system.

In a first aspect of the invention, the present invention relates to compounds of Formula (I):

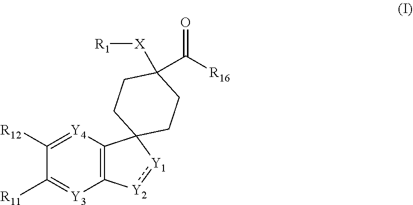

wherein:
X represents —S—, —O—, —CH₂— or —N(R₂)—,
Y₁ represents —C(R₄)(R₅)— or —N(R₆)—,
Y₂ represents —N(R₇)—, —C(R₈)(R₉)—, or —C(R₈)(R₉)—C(R₁₄)(R₁₅)—,
[symbol] means a single bond or a double bond,
Y₃ represents —C(R₁₀)— or —N—,
Y₄ represents —C(R₁₃)— or —N—,
R₁ represents an aryl group or a heteroaryl group,
R₂ represents a hydrogen atom or a linear or branched (C₁-C₆)alkyl group,
or the pair (R₁,R₂) together with the nitrogen atom to which they are attached forms a non-aromatic or aromatic mono- or bicyclic ring composed of from 5 to 12 ring members, which may contain in addition to the nitrogen a second heteroatom selected from oxygen, sulphur and nitrogen, wherein said ring may be substituted by from 1 to 2 groups representing a hydrogen atom, a halogen atom, or a linear or branched (C₁-C₆)alkyl group,
R₃ represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)(OR_{3A})₂, —W₁—SO₂—OR_{3A}, or —W₁—Cy₁, wherein:
   W₁ represents a bond or a linear or branched (C₁-C₄)alkylene group,
   R_{3A} and R_{3B} independently of one another, represent a hydrogen atom, a linear or branched (C₁-C₆)alkyl group, a linear or branched (C₁-C₆)alkoxy(C₁-C₆)alkyl group, or a cycloalkyl group,
   or the pair (R_{3A},R_{3B}) together with the nitrogen atom to which they are attached forms a non-aromatic ring composed of from 4 to 7 ring members, which may contain in addition to the nitrogen a second heteroatom selected from oxygen and nitrogen,
   Cy₁ represents an aryl group or a heteroaryl group,
R₄ represents a hydrogen atom, a halogen atom, a linear or branched (C₁-C₆)alkyl group, a linear or branched (C₁-C₆)alkyl group substituted by 2 linear or branched (C₁-C₆)alkoxy groups, a linear or branched (C₂-C₆)alkenyl group, a linear or branched (C₂-C₆)alkynyl group, a linear or branched (C₁-C₆)alkoxy group, a linear or branched (C₂-C₆)alkenyloxy group, a linear or branched (C₁-C₆)alkoxy(C₁-C₆)alkyl group, a linear or branched (C₁-C₆)alkoxy(C₂-C₆)alkenyl group, a linear or branched (C₁-C₆)alkoxy(C₁-C₆)haloalkyl group, a hydroxy group, a linear or branched (C₁-C₆)hydroxyalkyl group, a —W₂-Cy₂ group, a —W₃-L-Cy₃ group, a —W₄—NR_{4A}R_{4B} group, or a —CO—NR_{4C}R_{4D} group, wherein:
   W₂ represents a bond, a linear or branched (C₁-C₆)alkylene group, a linear or branched (C₂-C₆)alkenylene group, or a linear or branched (C₂-C₆)alkynylene group,
   W₃ represents a bond, a linear or branched (C₁-C₆)alkylene group, a linear or branched (C₂-C₆)alkenylene group, a linear or branched (C₂-C₈)alkynylene group, a linear or branched (C₁-C₆)alkoxylene group, a linear or branched (C₁-C₄)hydroxyalkylene group, a linear or branched (C₁-C₄)haloalkylene group, or a —CH₂—CH(R_{4E})—CH₂— group,
   W₄ represents a linear or branched (C₁-C₄)alkylene group,
   L represents —O—, —S—, or —SO₂—,
   R_{4A} and R_{4B} independently of one another represent a hydrogen atom, a linear or branched (C₁-C₆)alkyl group, an aryl group, a heteroaryl group, or an arylalkyl group,
R_{4C} and R_{4D} independently of one another represent a hydrogen atom, a linear or branched (C₁-C₆)alkyl group, an arylalkyl group, or a heteroarylalkyl group,
R_{4E} represents —Cy₄ or —CH₂—O—Cy₄,
   Cy₂ represents an aryl group, a heteroaryl group, a cycloalkyl group, a cycloalkenyl group, or a heterocycloalkyl group,
   Cy₃ represents an aryl group, a heteroaryl group, a cycloalkyl group, a heterocycloalkyl group, an arylalkyl group, or a heteroarylalkyl group,
   Cy₄ represents an aryl group, a heteroaryl group, an arylalkyl group, or a heterocycloalkylalkyl group,
R₅ represents a hydrogen atom or a linear or branched (C₁-C₆)alkyl group, or the pair (R₄,R₅) represents an oxo group, or a cycloalkylidene group, or the pair (R₄,R₅) together with carbon atoms to which they are attached forms a non-aromatic ring composed of from 3 to 6 ring members,
R₆ represents an aryl group, a —SO₂-aryl group, or a —W₅—O—Cy₅ group, wherein:
   W₅ represents a linear or branched (C₁-C₄)alkylene group,
   Cy₅ represents an aryl group, or a heteroaryl group,
R₇ represents a hydrogen atom, a linear or branched (C₁-C₆)alkyl group, an arylalkyl group, or a formyl group,
R₈ represents a hydrogen atom, or a linear or branched (C₁-C₆)alkyl group, or the pair (R₄,R₈) together with carbon atoms to which they are attached forms a non-aromatic or aromatic ring composed of from 3 to 7 ring members,
R₉ represents a hydrogen atom, or the pair (R₈,R₉) represents an oxo group,
R₁₀ represents a hydrogen atom, a halogen atom, or a linear or branched (C₁-C₆)alkyl group,
or the pair (R₇,R₁₀) together with the nitrogen atom to which they are attached forms a non-aromatic ring composed of from 4 to 7 ring members,
R₁₁ represents a hydrogen atom, a halogen atom, a linear or branched (C₁-C₆)alkyl group, or a linear or branched (C₁-C₆)alkoxy group,
R₁₂ represents a hydrogen atom, a halogen atom, a linear or branched (C₁-C₆)alkyl group, a linear or branched (C₁-C₆)alkenyl group, a linear or branched (C₁-C₆)alkynyl group, a linear or branched (C₁-C₆)alkoxy group, a linear or branched (C₁-C₆)alkenyloxy group, a linear or branched halo(C₁-C₆)alkyloxy group, a linear or branched (C₁-C₆)alkoxy(C₁-C₆)alkyl group, a linear or branched (C₁-C₆)alkoxy(C₁-C₆)alkoxy group, a hydroxy group, a linear or branched hydroxy(C₁-C₆)alkyl group, an acetyl group, a formyl group, a —CH₂—O-tetrahydrofuranyl group, —Cy₆, or —O—Cy₇, wherein:
   Cy₆ represents an aryl group, a heteroaryl group, a cycloalkyl group, an arylalkyl group, or an arylalkenyl group,
   Cy₇ represents an aryl group, cycloalkyl group, or a cycloalkylalkyl group,
or the pair (R₁₁,R₁₂) together with the carbon atoms to which they are attached forms a non-aromatic ring composed of from 5 to 8 ring members, which may contain 1 or 2 oxygen atoms wherein said ring may be substituted by R₁₈ and R₁₈′,
R₁₃ represents a hydrogen atom, a halogen atom, or a linear or branched (C₁-C₆)alkyl group,
R₁₄ and R₁₅ independently of one another, represent a hydrogen atom or a linear or branched (C₁-C₆)alkyl group,
R₁₆ represents a —O—R₃ group or a —NR₁₇R₁₇′ group,
R₁₇ and R₁₇′ independently of one another, represent a hydrogen atom, a linear or branched (C₁-C₆)alkyl group, a —SO₂—CF₃ group, or a —SO₂—CH₃ group,
R₁₈ and R₁₈′ independently of one another, represent a hydrogen atom, halogen atom, or a linear or branched (C₁-C₆)alkyl group,
or the pair (R₁₈,R₁₈′) together with the carbon atoms to which they are attached forms a non-aromatic ring composed of from 3 to 5 ring members,
   it being possible for the aryl, heteroaryl, cycloalkyl, cycloalkenyl, heterocycloalkyl, arylalkyl, arylalkenyl, heteroarylalkyl, cycloalkylalkyl, or heterocycloalkylalkyl groups so defined to be substituted by from 1 to 4 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, linear or branched (C₁-C₆)alkoxy(C₁-C₆)alkoxy, a linear or branched halo (C₁-C₆)alkyloxy group, hydroxy, a linear or branched hydroxy(C₁-C₆)alkyl group, cyano, oxo, —NR′R″, —C(O)—OR′, cyclopropyl, 2,2-dimethylcyclopropyl, phenyl, pyridinyl, benzyl, (2,3,6-trifluorophenyl)methyl, —CH₂-pyridinyl, —O-phenyl, —O-benzyl, —O-pyridinyl, —O—CH₂-cyclopropyl, —O—CH₂-pyridinyl, aryloxyalkyl or heteroaryloxyalkyl, wherein R′ and R″ independently of one another represent a hydrogen atom or linear or branched (C₁-C₆)alkyl,
   their enantiomers and diastereoisomers, and addition salts thereof with a pharmaceutically acceptable acid or base.

In another aspect, the invention provides compounds of Formula (I) as described herein, for use in the treatment of cancer, autoimmune diseases and the disease of immune system.

In a further aspect, the invention provides a pharmaceutical composition comprising the compounds of Formula (I) as described herein, and at least one pharmaceutically acceptable excipient.

### Definitions

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc.

Among the pharmaceutically acceptable bases there may be mentioned, without implying any limitation, sodium hydroxide, potassium hydroxide, triethylamine, tert-butylamine, etc.

“aryl” means a monocyclic or a fused bicyclic group composed of from 5 to 10 ring members, having at least one aromatic moiety. Among the aryl groups, there may be mentioned, without implying any limitation, phenyl, indanyl, naphthyl, etc. In a particular embodiment, an aryl group can be deuterated, more particularly a phenyl group can be tetradeuterated.

“heteroaryl” means a monocyclic, a fused bicyclic, or a bridged bicyclic group composed of from 5 to 12 ring members, having at least one aromatic moiety and containing from 1 to 3 heteroatoms selected from oxygen, sulphur and nitrogen. Among the heteroaryl groups, there may be mentioned, without implying any limitation, furyl, thienyl, thiazolyl, isoxazolyl, pyrazolyl, pyridinyl (also known as pyridyl), pyrimidinyl, pyridinonyl, indolyl, dihydroindolyl, indazolyl, tetrahydroindazolyl, benzofuranyl, dihydrobenzofuranyl, benzimidazolyl, benzopyranyl, benzodioxolyl, quinolinyl, dihydroquinolinyl, tetrahydroquinolinyl, tetrahydroquinazolinyl, pyrrolopyridinyl, thienopyrimidinyl, furopyridinyl, cyclopentapyridinyl, cyclopentapyrimidinyl, benzothiazolyl, hexahydropentalenopyridinyl, cycloheptapyridinyl, pyranopyridinyl, tetrahydronaphthyridinyl, tetrahydro-5,8-ethanoquinolinyl, pyrrolyl, isothiazolyl, oxazolyl, imidazolyl, pyrazinyl, pyridazinyl, dihydroisoindolyl, dihydrocyclopentathienyl, benzothienyl, tetrahydrobenzothienyl, imidazopyridinyl, benzotriazolyl, dihydrobenzodioxinyl, isoquinolinyl, tetrahydroisoquinolinyl, quinoxalinyl, dihydroquinoxalinyl, dihydrothienodioxinyl, quinazolinonyl, pyrrolopyridazinyl, dihydropyrrolizinyl, tetrahydroindolizinyl, etc.

“cycloalkyl” means a monocyclic, a fused bicyclic, or a bridged bicyclic non-aromatic carbocyclic group composed of from 3 to 7 ring members. Among the cycloalkyl groups, there may be mentioned, without implying any limitation, cyclopropyl, cyclobutyl, cyclopentyl, cyclohexyl, etc.

“cycloalkenyl” means a monocyclic, a fused bicyclic, or a bridged bicyclic non-aromatic carbocyclic group composed of from 3 to 7 ring members and having one or more double bonds. Among the cycloalkenyl groups, there may be mentioned, without implying any limitation, cyclohexenyl, bicyclo[2.2.1]heptenyl, cyclopentenyl, etc.

“heterocycloalkyl” means a monocyclic or fused bicyclic non-aromatic group composed of from 3 to 10 ring members, containing from 1 to 3 heteroatoms selected from oxygen, sulphur and nitrogen, and may have one double bond. Among the heterocycloalkyl groups, there may be mentioned, without implying any limitation, azetidinyl, tetrahydropyranyl, tetrahydropyridinyl, piperidinyl, piperazinyl, morpholinyl, pyrrolidinyl, etc.

“alkylene” or “(C₁-C₆)alkylene” means a divalent linear or branched, saturated hydrocarbon radical having from 1 to 6 carbon atoms. Among the alkylene radicals, there may be mentioned, without implying any limitation, —CH₂—, —(CH₂)₂—, —(CH₂)₃—, —(CH₂)₄—, —CH(CH₃)—, —CH₂—CH(CH₃)—, —CH(CH₃)—CH₂—, —CH₂—CH(CH₃)—CH₂—, —CH₂—CH(CH₂—CH₃)—CH₂—, —CH₂—CH[CH(CH₃)₂]—CH₂—, —CH₂—C(CH₃)₂—CH₂—, —CH₂—CH(CH₃)—CH(CH₃)—, etc.

“alkenylene” or “(C₂-C₆)alkenylene” means a divalent linear or branched, hydrocarbon radical having from 2 to 6 carbon atoms and one or more double bonds. More preferably, “alkenylene” refers to a divalent linear or branched hydrocarbon chain having 2 to 6 carbon atoms and one double bond. Among the alkenylene radicals, there may be mentioned, without implying any limitation, —CH═CH—, —CH═CH—CH₂—, —CH₂—CH═CH—, etc.

“alkynylene” or “(C₂-C₈)alkylene” means a divalent linear or branched, hydrocarbon radical having from 2 to 8 carbon atoms and one or more triple bonds. More preferably, “alkynylene” refers to a divalent linear or branched hydrocarbon chain having 2 to 8 carbon atoms and one triple bond. Among the alkynylene radicals, there may be mentioned, without implying any limitation, —C≡C—, —C≡C—CH₂—, —CH₂—C≡C—, —CH₂—CH(C≡C—(CH₂)₂—CH₃)—CH₂—, etc.

“hydroxyalkylene” or “(C₁-C₄)hydroxyalkylene” means a divalent linear or branched, saturated hydrocarbon radical having from 1 to 4 carbon atoms, and one or more hydroxy groups. More preferably, “hydroxyalkylene” refers to a divalent linear or branched hydrocarbon chain having 1 to 4 carbon atoms and one hydroxy group. Among the hydroxyalkylene radicals, there may be mentioned, without implying any limitation, —CH(OH)—, —CH₂—CH(OH)—, —CH(OH)—CH₂—, —CH₂—CH(CH₂—OH)—CH₂—, etc.

“haloalkylene” or “(C₁-C₄)haloalkylene” means a divalent linear or branched, saturated hydrocarbon radical having from 1 to 4 carbon atoms, and one or more halogens atoms. More preferably, “haloalkylene” refers to a divalent linear or branched hydrocarbon chain having 1 to 4 carbon atoms and one or more halogens atoms selected from fluorine, chlorine or bromine, more preferably fluorine. Among the haloalkylene radicals, there may be mentioned, without implying any limitation, —CHF—, —CF₂—, —CH₂—CHF—, —CHF—CH₂—, —CH₂—CF₂—CH₂—, —CH₂—CHF—CH₂—, —CH₂—CH(CH₂F)—CH₂—, —CH₂—CH(CHF₂)—CH₂—, —CH₂—CF(CH₃)—CH₂—, etc.

“alkoxylene” or “(C₁-C₆)alkoxylene” means a divalent linear or branched, saturated hydrocarbon radical having from 1 to 6 carbon atoms, and one or more oxygen atoms. More preferably, “alkoxylene” refers to a divalent linear or branched hydrocarbon chain having 1 to 6 carbon atoms and one oxygen atom. Among the alkoxylene radicals, there may be mentioned, without implying any limitation, —CH₂—O—, —CH₂OCH₂—, —O—(CH₂)₃—, —CH₂—CH(CH₂O—CH₃)—CH₂—, etc.

The term “cycloalkylalkyl” used herein refers to a linear or branched —(C₁-C₄)alkyl-Z₁ group, wherein “Z₁” is a cycloalkyl group, preferably a cyclopropyl group, which can be substituted by 0, 1, or 2 substituents independently selected from halogen, (C₁-C₆)alkyl, or (C₁-C₆)alkoxy, preferably fluorine, chlorine, methyl, or methoxy. Among the cycloalkylalkyl groups, there may be mentioned, without implying any limitation, —CH₂-cyclopropyl, —(CH₂)₂-cyclopropyl, etc.

The term “arylalkyl” used herein refers to a linear or branched —(C₁-C₄)alkyl-Z₂ group, wherein “Z₂” is an aryl group, preferably a phenyl group, which can be substituted by 0, 1, 2, or 3 substituents independently selected from halogen, (C₁-C₆)alkyl, or (C₁-C₆)alkoxy, preferably fluorine, chlorine, methyl, or methoxy. Among the arylalkyl groups, there may be mentioned, without implying any limitation, —CH₂-phenyl (also known as benzyl), —(CH₂)₂-phenyl, —(CH₂)₃-phenyl, —CH(CH₃)-phenyl, etc.

The term “arylalkenyl” used herein refers to a linear or branched —(C₂-C₄)alkenyl-Z₃ group, wherein “Z₃” is an aryl group, preferably a phenyl group, which can be substituted by 0, 1, or 2 substituents independently selected from halogen, (C₁-C₆)alkyl, or (C₁-C₆)alkoxy, preferably fluorine, chlorine, methyl, or methoxy. Among the arylalkenyl groups, there may be mentioned, without implying any limitation, —CH═CH-phenyl, —CH═CH—CH₂-phenyl, etc.

The term “heteroarylalkyl” used herein refers to a linear or branched —(C₁-C₄)alkyl-Z₄ group, wherein “Z₄” is a heteroaryl group, preferably a pyridinyl group, which can be substituted by 0, 1, or 2 substituents independently selected from halogen, (C₁-C₆)alkyl, or (C₁-C₆)alkoxy, preferably fluorine, chlorine, methyl, or methoxy. Among the heteroarylalkyl groups, there may be mentioned, without implying any limitation, —CH₂-pyridinyl, —(CH₂)₂-pyridinyl, —(CH₂)₃-pyridinyl, etc.

The term “heterocycloalkylalkyl” used herein refers to a linear or branched —(C₁-C₄)alkyl-Z₅ group, wherein “Z₅” is a heterocycloalkyl group, preferably a morpholinyl group, which can be substituted by 0, 1, or 2 substituents independently selected from halogen, (C₁-C₆)alkyl, or (C₁-C₆)alkoxy, preferably fluorine, chlorine, methyl, or methoxy. Among the heterocycloalkylalkyl groups, there may be mentioned, without implying any limitation, —CH₂-morpholinyl, —(CH₂)₂-morpholinyl, etc.

The term “aryloxyalkyl” used herein refers to a linear or branched —(C₁-C₄)alkyl-O—Z₆, wherein “Z₆” is an aryl group, preferably a phenyl group, which can be substituted by 0, 1, or 2 substituents independently selected from halogen, (C₁-C₆)alkyl, or (C₁-C₆)alkoxy, preferably fluorine, chlorine, methyl, or methoxy. Among the aryloxyalkyl groups, there may be mentioned, without implying any limitation, —CH₂—O-phenyl, —(CH₂)₂—O-phenyl, etc.

The term “heteroaryloxyalkyl” used herein refers to a linear or branched —(C₁-C₄)alkyl-O—Z₇, wherein “Z₇” is a heteroaryl group, preferably a pyridinyl group or a thienopyridinyl group, each can be substituted by 0, 1, or 2 substituents independently selected from halogen, (C₁-C₆)alkyl, or (C₁-C₆)alkoxy, preferably fluorine, chlorine, methyl, or methoxy. Among the heteroaryloxyalkyl groups, there may be mentioned, without implying any limitation, —CH₂—O-pyridinyl, —(CH₂)₂—O-pyridinyl, —CH₂—O-thienopyridinyl, —(CH₂)₂—O-thienopyridinyl, etc.

“spirocyclohexane compounds” or “spirocyclohexane derivatives” or “spirocyclohexane scaffolds” mean compounds having at least two molecular rings with only one common atom (Moss, Pure Appl. Chem. 1999, 71, 531-558). The common atom that connects the two rings is called the spiro atom which is a quaternary carbon in the present case. For compounds according to the invention, the 1,1,4,4-tetrasubstituted spirocyclohexane allows the formation of two diastereoisomers which are represented as follows:

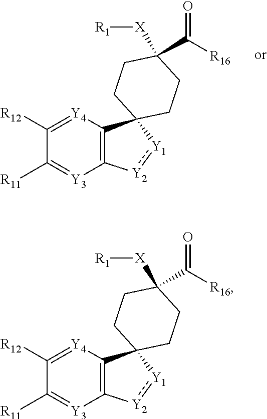

or represented as follows:

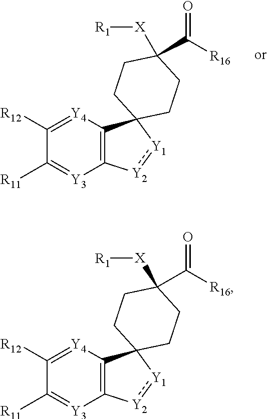

wherein the —C(═O)—R₁₆ group is located to the same side of the benzene-type ring (as shown above on the left), or wherein the —X—R₁ group is located to the same side of the benzene-type ring (as shown above on the right). Preferred diastereoisomer of spirocyclohexane derivatives according to the invention is represented as follows:

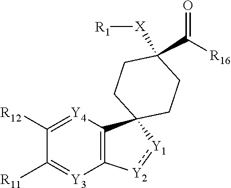

or represented as follows:

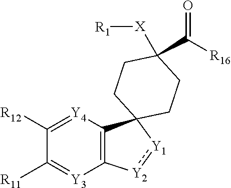

wherein the —C(═O)—R₁₆ group is located to the same side of the benzene-type ring.

The symbol “*” close to two substituted asymmetric carbon atoms (chiral centers) drawn on a molecule scheme means relative stereochemistry. The real configuration of these chiral centers can be either the one drawn or the one where all stereocenters with “*” have opposite configuration compared to the drawn. For example, rac-(5R,8S)-4-chloro-5-methyl-5,6,7,8-tetrahydroquinolin-8-ol

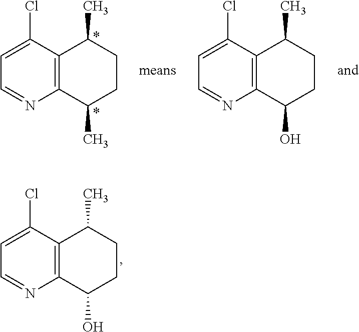

while (5R*,8S*)-4-chloro-5-methyl-5,6,7,8-tetrahydroquinolin-8-ol, enantiomer 1

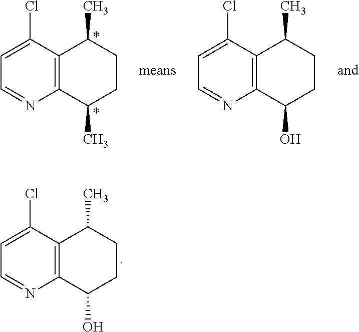

Among the pharmaceutical compositions according to the invention there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules. The pharmaceutical compositions according to the invention comprise one or more excipients or carriers selected from diluents (such as lactose, dextrose, sucrose, mannitol, sorbitol, cellulose, glycerol . . . ), lubricants (such as silica, talc, stearic acid and its magnesium and calcium salts, polyethylene glycol . . . ), binders (such as magnesium aluminum silicate, starch, gelatin, tragacanth, methylcellulose, sodium carboxymethylcellulose and polyvinylpyrrolidone . . . ), disintegration agents (such as agar, alginic acid and its sodium salt, effervescent mixtures . . . ), stabilizers, preservatives, absorbents, colorants, sweeteners, flavorings, etc. The administration route is preferably the oral route or the intravenous route, and the corresponding pharmaceutical compositions may allow the instantaneous or delayed release of the active ingredients.

Among the combinations of a compound of Formula (I) with an anticancer agent according to the invention, there may be mentioned more especially those that are suitable for a simultaneous administration or a sequential administration. The combinations according to the invention comprise a compound of Formula (I) combined to anti-cancer agents selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies. The compounds of the combination may moreover be administered in the form of two separate pharmaceutical compositions, each containing one of the active ingredients, or in the form of a single pharmaceutical composition, in which the active ingredients are in admixture.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both.

Among the cancer treatments envisaged there may be mentioned, without implying any limitation, the treatment of haematological malignancies and solid tumors. Haematological malignancies include myeloma, especially multiple myeloma, lymphoma, especially Non-Hodgkin Lymphoma (NHL) and Diffuse Large B-cell Lymphoma (DLBCL), and leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML). Solid tumors include the bladder, brain, breast, uterus, oesophagus and liver cancers, colorectal cancer, renal cancer, melanoma, ovarian cancer, prostate cancer, pancreatic cancer and lung cancer, especially non-small-cell lung cancer and small-cell lung cancer.

Among the treatments of autoimmune diseases envisaged there may be mentioned, without implying any limitation, the treatment of rheumatoid arthritis (RA) and systemic lupus erythematosus (SLE).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts. A suitable daily dose of a compound of the invention will depend upon the factors described above and may range from 0.01 mg to 2.5 g per day in one or more administration(s).

### DETAILED DESCRIPTION

Advantageously, X represents —O— or —N(R₂)—. Preferably, X represents —N(R₂)—. More preferably, X represents —NH—.

In one preferred embodiment, [symbol] represents a single bond.

In one another preferred embodiment, when [symbol] represents a single bond, independently of one another, Y₁ represents —C(R₄)(R₅)— or —N(R₆)— and Y₂ represents —N(R₇)—, —C(R₈)(R₉)—, or —C(R₈)(R₉)—C(R₁₄)(R₁₅)—.

Preferably, Y₁ represents —C(R₄)(R₅)—. More preferably, Y₁ represents —N(R₆)—.

In one preferred embodiment, Y₂ represents —N(R₇)—.

In one another preferred embodiment, Y₂ represents —C(R₈)(R₉)—.

In one preferred embodiment, Y₂ represents —C(R₈)(R₉)—C(R₁₄)(R₁₅)—.

Preferably, Y₁ represents —C(R₄)(R₅)— and Y₂ represents —C(R₈)(R₉)—.

In one another embodiment, Y₁ represents —C(R₄)(R₅)— and Y₂ represents —N(R₇)—.

In one another embodiment, Y₁ represents —N(R₆)— and Y₂ represents —C(R₈)(R₉)—.

In another embodiment, Y₁ represents —C(R₄)(R₅)— and Y₂ represents —C(R₈)(R₉)—C(R₁₄)(R₁₅)—.

Preferably, when [symbol] represents a double bond, independently of one another, Y₁ represents —C(R₄)— and Y₂ represents —N═ or —C(R₈)—.

In a preferred embodiment, Y₁-Y₂ represents —C(R₄)═N—.

In another embodiment, Y₁-Y₂ represents —C(R₄)═C(R₈)—.

In preferred embodiment, Y₃ represents —C(R₁₀)—.

Preferably, Y₄ represents —C(R₁₃)—.

Advantageously, Y₃ represents —C(R₁₀)— and Y₄ represents —C(R₁₃)—.

An advantageous possibility consists of compounds of Formula (I-a):

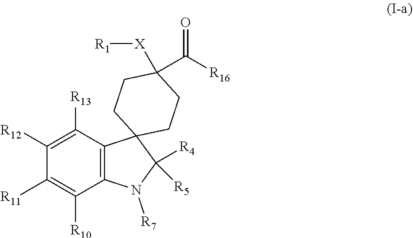

wherein R₁, R₄, R₅, R₇, R₁₀, R₁₁, R₁₂, R₁₃, R₁₆ and X are as defined for Formula (I).

Another advantageous possibility consists of compounds of Formula (I-a):

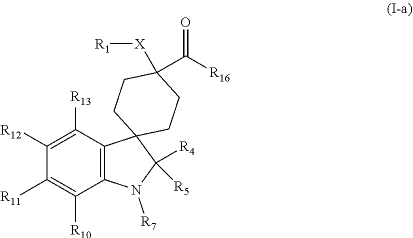

wherein:
X represents —N(R₂)—,
R₁ represents an aryl group,
R₂, R₃, R₁₁ and R₁₃ represent a hydrogen atom,
R₁₆ represents a —O—R₃ group,
R₄ represents a hydrogen atom, a linear or branched (C₁-C₆)alkyl group, or a —W₂—Cy₂ group, wherein:
   W₂ represents a linear or branched (C₁-C₆)alkylene group,
   Cy₂ represents an aryl group,
R₅ represents a hydrogen atom,
or the pair (R₄,R₅) represents an oxo group,
R₇ represents a hydrogen atom, a linear or branched (C₁-C₆)alkyl group, an arylalkyl group, or a formyl group,
R₁₀ represents a hydrogen atom, or a linear or branched (C₁-C₆)alkyl group,
or the pair (R₇,R₁₀) together with the nitrogen atom to which they are attached forms a non-aromatic ring composed of from 4 to 7 ring members,
R₁₂ represents a hydrogen atom, a halogen atom, a linear or branched (C₁-C₆)alkyl group, a linear or branched (C₁-C₆)alkenyl group, a linear or branched (C₁-C₆)alkynyl group, or —Cy₆, wherein:
   Cy₆ represents an aryl group, a cycloalkyl group, an arylalkyl group, or an arylalkenyl group.

Another advantageous possibility consists of compounds of Formula (I-b):

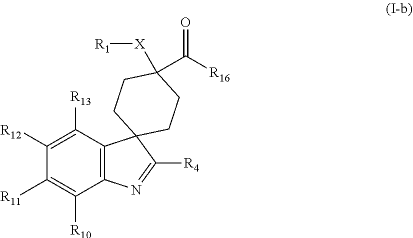

wherein R₁, R₄, R₁₀, R₁₁, R₁₂, R₁₃, R₁₆ and X are as defined for Formula (I).

Another advantageous possibility consists of compounds of Formula (I-b):

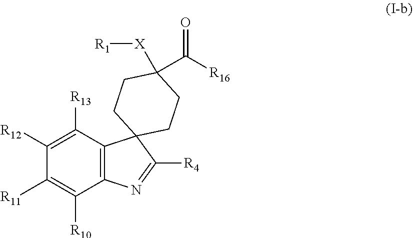

wherein:
X represents —N(R₂)—,
R₁ represents an aryl group,
R₂, R₃, R₁₀, R₁₁ and R₁₃ represent a hydrogen atom,
R₁₆ represents a —O—R₃ group,
R₄ represents a linear or branched (C₁-C₆)alkyl group,
R₁₂ represents a hydrogen atom or a halogen atom.

Another advantageous possibility consists of compounds of Formula (I-c):

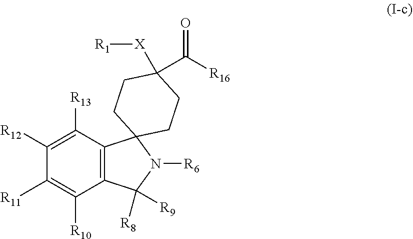

wherein R₁, R₆, R₈, R₉, R₁₀, R₁₁, R₁₂, R₁₃, R₁₆ and X are as defined for Formula (I).

Another advantageous possibility consists of compounds of Formula (I-c):

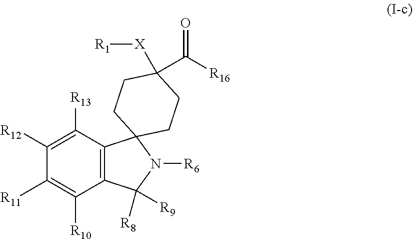

wherein:
X represents —N(R₂)—,
R₁ represents an aryl group,
R₁₆ represents a —O—R₃ group,
R₂, R₃, R₈, R₉, R₁₀ and R₁₃ represent a hydrogen atom,
or the pair (R₈,R₉) represents an oxo group,
R₆ represents an aryl group, a —SO₂-aryl group, or a —W₅—O—Cy₅ group, wherein:
   W₅ represents a linear or branched (C₁-C₄)alkylene group,
   Cy₅ represents an aryl group, or a heteroaryl group,
R₁₁ represents a hydrogen atom, a halogen atom, or a linear or branched (C₁-C₆)alkyl group,
R₁₂ represents a hydrogen atom, a linear or branched (C₁-C₆)alkoxy group, or a linear or branched (C₁-C₆)alkoxy(C₁-C₆)alkyl group,
or the pair (R₁₁,R₁₂) together with the carbon atoms to which they are attached forms a non-aromatic ring composed of from 5 to 8 ring members, which may contain 1 or 2 oxygen atoms.

Another advantageous possibility consists of compounds of Formula (I-d):

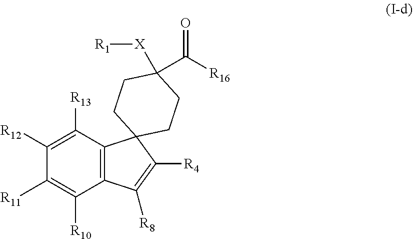

wherein R₁, R₄, R₈, R₁₀, R₁₁, R₁₂, R₁₃, R₁₆ and X are as defined for Formula (I).

Another advantageous possibility consists of compounds of Formula (I-d):

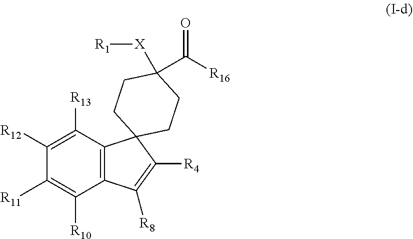

wherein:
X represents —N(R₂)—,
R₁ represents an aryl group or a heteroaryl group,
R₂ represents a hydrogen atom or a linear or branched (C₁-C₆)alkyl group,
R₁₆ represents a —O—R₃ group,
R₃ represents a hydrogen atom,
R₄ represents a hydrogen atom, a halogen atom, a linear or branched (C₁-C₆)alkyl group, a linear or branched (C₁-C₆)alkyl group substituted by 2 linear or branched (C₁-C₆)alkoxy groups, a linear or branched (C₂-C₆)alkenyl group, a linear or branched (C₁-C₆)alkoxy(C₂-C₆)alkenyl group, a —W₂-Cy₂ group, a —W₃-L-Cy₃ group, a —W₄—NR_{4A}R_{4B} group, or a —CO—NR_{4C}R_{4D} group, wherein:
   W₂ represents a bond, a linear or branched (C₁-C₆)alkylene group, a linear or branched (C₂-C₆)alkenylene group, or a linear or branched (C₂-C₆)alkynylene group,
   W₃ represents a bond, a linear or branched (C₁-C₆)alkylene group, a linear or branched (C₂-C₈)alkynylene group, a linear or branched (C₁-C₆)alkoxylene group, a linear or branched (C₁-C₄)hydroxyalkylene group, a linear or branched (C₁-C₄)haloalkylene group, or a —CH₂—CH(R_{4E})—CH₂— group,
   W₄ represents a linear or branched (C₁-C₄)alkylene group,
   L represents —O—,
   R_{4A} and R_{4B} independently of one another represent a hydrogen atom, a linear or branched (C₁-C₆)alkyl group, an aryl group, or an arylalkyl group,
   R_{4C} and R_{4D} independently of one another represent a hydrogen atom, a linear or branched (C₁-C₆)alkyl group, an arylalkyl group, or a heteroarylalkyl group,
   R_{4E} represents —Cy₄ or —CH₂—O—Cy₄,
   Cy₂ represents an aryl group, a heteroaryl group, a cycloalkyl group, a cycloalkenyl group, or a heterocycloalkyl group,
   Cy₃ represents an aryl group, a heteroaryl group, an arylalkyl group, or a heteroarylalkyl group,
   Cy₄ represents a heteroaryl group, or an arylalkyl group,
R₈ represents a hydrogen atom, or a linear or branched (C₁-C₆)alkyl group,
R₁₀ represents a hydrogen atom, a halogen atom, or a linear or branched (C₁-C₆)alkyl group,
R₁₁ represents a hydrogen atom, a halogen atom, a linear or branched (C₁-C₆)alkyl group, or a linear or branched (C₁-C₆)alkoxy group,
R₁₂ represents a hydrogen atom, a halogen atom, a linear or branched (C₁-C₆)alkyl group, a linear or branched (C₁-C₆)alkoxy group, a linear or branched hydroxy(C₁-C₆)alkyl group, or an acetyl group,
R₁₃ represents a hydrogen atom, a halogen atom, or a linear or branched (C₁-C₆)alkyl group.

Another advantageous possibility consists of compounds of Formula (I-e):

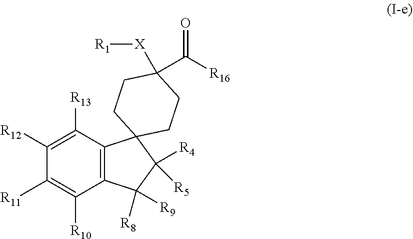

wherein R₁, R₄, R₅, R₈, R₉, R₁₀, R₁₁, R₁₂, R₁₃, R₁₆ and X are as defined for Formula (I).

Another advantageous possibility consists of compounds of Formula (I-e):

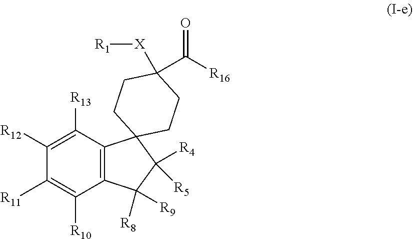

wherein:
X represents —S—, —O—, —CH₂— or —N(R₂)—,
R₁ represents an aryl group or a heteroaryl group,
R₂ represents a hydrogen atom,
or the pair (R₁,R₂) together with the nitrogen atom to which they are attached forms a non-aromatic or aromatic mono- or bicyclic ring composed of from 5 to 12 ring members, which may contain in addition to the nitrogen a second heteroatom selected from oxygen, sulphur and nitrogen, wherein said ring may be substituted by from 1 to 2 groups representing a hydrogen atom, a halogen atom, or a linear or branched (C₁-C₆)alkyl group,
R₃ represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)(OR_{3A})₂, —W₁—SO₂—OR_{3A}, or —W₁—Cy₁, wherein:
   W₁ represents a bond or a linear or branched (C₁-C₄)alkylene group,
   R_{3A} and R_{3B} independently of one another, represent a hydrogen atom, a linear or branched (C₁-C₆)alkyl group, a linear or branched (C₁-C₆)alkoxy(C₁-C₆)alkyl group, or a cycloalkyl group,
   or the pair (R_{3A},R_{3B}) together with the nitrogen atom to which they are attached forms a non-aromatic ring composed of from 4 to 7 ring members, which may contain in addition to the nitrogen a second heteroatom selected from oxygen and nitrogen,
   Cy₁ represents an aryl group or a heteroaryl group,
R₄ represents a hydrogen atom, a linear or branched (C₁-C₆)alkyl group, a linear or branched (C₂-C₆)alkenyloxy group, a linear or branched (C₁-C₆)alkoxy(C₁-C₆)alkyl group, a linear or branched (C₁-C₆)alkoxy(C₁-C₆)haloalkyl group, a —W₂—Cy₂ group, a —W₃-L-Cy₃ group, a —W₄—NR_{4A}R_{4B} group, or a —CO—NR_{4C}R_{4D} group, wherein:
   W₂ represents a bond, a linear or branched (C₁-C₆)alkylene group,
   W₃ represents a bond, a linear or branched (C₁-C₆)alkylene group, a linear or branched (C₁-C₆)alkoxylene group, a linear or branched (C₁-C₄)hydroxyalkylene group, a linear or branched (C₁-C₄)haloalkylene group, or a —CH₂—CH(R_{4E})—CH₂— group,
   W₄ represents a linear or branched (C₁-C₄)alkylene group,
   L represents —O—, —S—, or —SO₂—,
   R_{4A} and R_{4B} independently of one another represent a hydrogen atom, or a heteroaryl group,
   R_{4C} and R_{4D} independently of one another represent a hydrogen atom, a linear or branched (C₁-C₆)alkyl group, an arylalkyl group, or a heteroarylalkyl group,
   R_{4E} represents —Cy₄ or —CH₂—O—Cy₄,
   Cy₂ represents an aryl group, a heteroaryl group, a cycloalkyl group, or a heterocycloalkyl group,
   Cy₃ represents an aryl group, a heteroaryl group, a heterocycloalkyl group, an arylalkyl group, or a heteroarylalkyl group,
   Cy₄ represents an aryl group, a heteroaryl group, an arylalkyl group, or a heterocycloalkylalkyl group,
R₅ represents a hydrogen atom or a linear or branched (C₁-C₆)alkyl group,
or the pair (R₄,R₅) represents a cycloalkylidene group,
or the pair (R₄,R₅) together with carbon atoms to which they are attached forms a non-aromatic ring composed of from 3 to 7 ring members,
R₈ represents a hydrogen atom, or a linear or branched (C₁-C₆)alkyl group,
or the pair (R₄,R₈) together with carbon atoms to which they are attached forms a non-aromatic or aromatic ring composed of from 3 to 7 ring members,
R₉ represents a hydrogen atom,
R₁₀ represents a hydrogen atom, a halogen atom, or a linear or branched (C₁-C₆)alkyl group,
R₁₁ represents a hydrogen atom, a halogen atom, a linear or branched (C₁-C₆)alkyl group, or a linear or branched (C₁-C₆)alkoxy group,
R₁₂ represents a hydrogen atom, a halogen atom, a linear or branched (C₁-C₆)alkyl group, a linear or branched (C₁-C₆)alkoxy group, a linear or branched (C₁-C₆)alkenyloxy group, a linear or branched halo(C₁-C₆)alkyloxy group, a linear or branched (C₁-C₆)alkoxy(C₁-C₆)alkyl group, a linear or branched (C₁-C₆)alkoxy(C₁-C₆)alkoxy group, a hydroxy group, a linear or branched hydroxy(C₁-C₆)alkyl group, an acetyl group, a formyl group, a —CH₂—O-tetrahydrofuranyl group, —Cy₆, or —O—Cy₇, wherein:
   Cy₆ represents an aryl group, a heteroaryl group, a cycloalkyl group, or an arylalkyl group,
   Cy₇ represents an aryl group, a cycloalkyl group, or a cycloalkylalkyl group, or the pair (R₁₁,R₁₂) together with the carbon atoms to which they are attached forms a non-aromatic ring composed of from 5 to 8 ring members, which may contain 1 or 2 oxygen atoms wherein said ring may be substituted by R₁₈ and R₁₈′,
R₁₃ represents a hydrogen atom, a halogen atom, or a linear or branched (C₁-C₆)alkyl group,
R₁₆ represents a —O—R₃ group or a —NR₁₇R₁₇′ group,
R₁₇ represents a hydrogen atom, a linear or branched (C₁-C₆)alkyl group, a —SO₂—CF₃ group, or a —SO₂—CH₃ group,
R₁₇′ represents a hydrogen atom, or a linear or branched (C₁-C₆)alkyl group,
R₁₈ and R₁₈′ independently of one another, represent a hydrogen atom, a halogen atom, or a linear or branched (C₁-C₆)alkyl group,
or the pair (R₁₈,R₁₈′) together with the carbon atoms to which they are attached forms a cyclopropyl ring or a cyclobutyl ring.

Preferably, compounds of Formula (I-e) are:

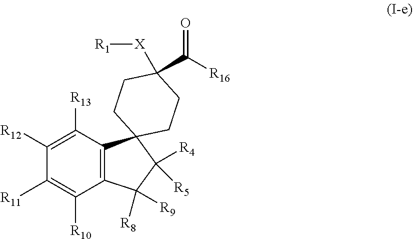

wherein R₁, R₄, R₅, R₈, R₉, R₁₀, R₁₁, R₁₂, R₁₃, R₁₆ and X are as defined previously.

More preferably, compounds of Formula (I-e) are:

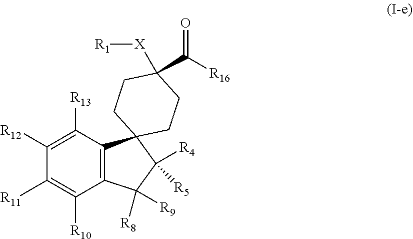

wherein R₁, R₄, R₅, R₈, R₉, R₁₀, R₁₁, R₁₂, R₁₃, R₁₆ and X are as defined previously.

Another advantageous possibility consists of compounds of Formula (I-f):

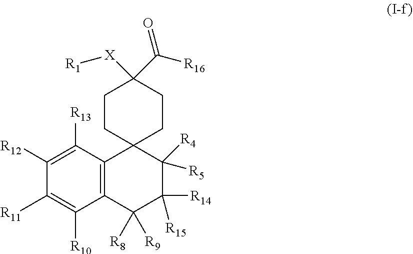

wherein R₁, R₄, R₅, R₈, R₉, R₁₀, R₁₁, R₁₂, R₁₃, R₁₄, R₁₅, R₁₆ and X are as defined for Formula (I).

Another advantageous possibility consists of compounds of Formula (I-f):

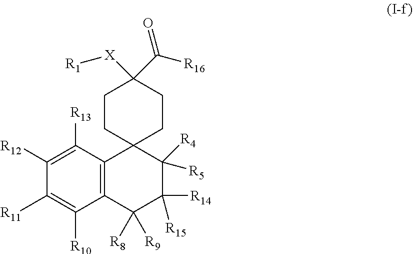

wherein:
X represents —N(R₂)—,
R₁ represents an aryl group,
R₁₆ represents a —O—R₃ group,
R₂, R₃, R₄, R₅, R₈, R₉, R₁₀, R₁₁, R₁₂, R₁₃, R₁₄ and R₁₅ represent a hydrogen atom.

Another advantageous possibility consists of compounds of Formula (I-g):

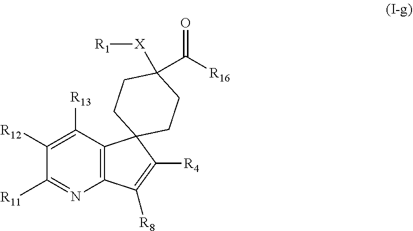

wherein R₁, R₄, R₈, R₁₁, R₁₂, R₁₃, R₁₆ and X are as defined for Formula (I).

Another advantageous possibility consists of compounds of Formula (I-g):

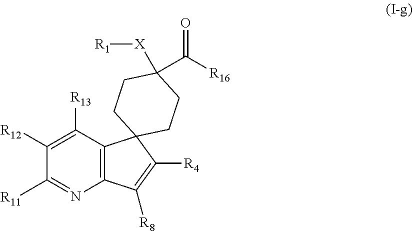

wherein:
X represents —N(R₂)—,
R₁ represents an aryl group,
R₁₆ represents a —O—R₃ group,
R₂, R₃, R₄, R₈, R₁₁, R₁₂ and R₁₃ represent a hydrogen atom.

Another advantageous possibility consists of compounds of Formula (I-h):

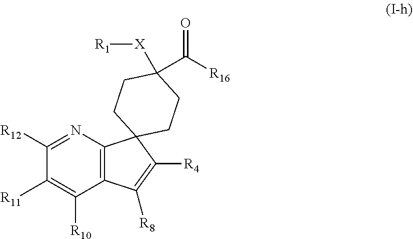

wherein R₁, R₄, R₈, R₉, R₁₀, R₁₁, R₁₂, R₁₆ and X are as defined for Formula (I).

Another advantageous possibility consists of compounds of Formula (I-h):

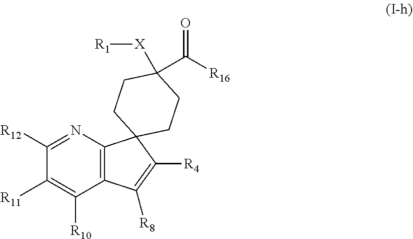

wherein:
X represents —N(R₂)—,
R₁ represents an aryl group,
R₁₆ represents a —O—R₃ group,
R₂, R₃, R₄, R₈, R₁₀, R₁₁ and R₁₂ represent a hydrogen atom.

In particular embodiments of compounds of Formulae (I-a), (I-b), (I-c), (I-d), (I-e), (I-f), (I-g) and (I-h) above-mentioned, it being possible for the aryl, heteroaryl, cycloalkyl, cycloalkenyl, heterocycloalkyl, arylalkyl, arylalkenyl, heteroarylalkyl, cycloalkylalkyl, or heterocycloalkylalkyl groups so defined to be substituted by from 1 to 4 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, linear or branched (C₁-C₆)alkoxy(C₁-C₆)alkoxy, a linear or branched halo(C₁-C₆)alkyloxy group, hydroxy, a linear or branched hydroxy(C₁-C₆)alkyl group, cyano, oxo, —NR′R″, —C(O)—OR′, cyclopropyl, 2,2-dimethylcyclopropyl, phenyl, pyridinyl, benzyl, (2,3,6-trifluorophenyl)methyl, —CH₂-pyridinyl, —O-phenyl, —O-benzyl, —O-pyridinyl, —O—CH₂-cyclopropyl, —O—CH₂-pyridinyl, aryloxyalkyl or heteroaryloxyalkyl, wherein R′ and R″ independently of one another represent a hydrogen atom or linear or branched (C₁-C₆)alkyl.

In a preferred embodiment of the invention, the present invention relates to compounds of Formula (I) wherein R₁ represents an aryl group, more preferably a phenyl group.

Preferably, R₁ represents an aryl group, more preferably a phenyl group, which is substituted by from 1 to 3 groups selected from halogen, linear or branched (C₁-C₆)alkyl, linear or branched halo(C₁-C₆)alkyl, linear or branched (C₁-C₆)alkoxy, cyano, or hydroxy.

More preferably, R₁ represents an aryl group, preferably a phenyl group, which is substituted by from 1 to 3 groups selected from fluorine, chlorine, bromine, methyl, difluoromethyl, trifluoromethyl, methoxy, cyano, or hydroxy.

Preferably, R₁ represents a 3-chloro-phenyl group, a 3-chloro-4-fluoro-phenyl group, a 3-chloro-2-fluoro-phenyl group, or a 3-chloro-2-methyl-phenyl group.

Preferably, R₁ represents a 3-chloro-phenyl group.

R₁ preferably represents a deuterated aryl group, preferably a deuterated phenyl group.

In a preferred embodiment, R₁ represents a heteroaryl group, preferably a pyridinyl group, more preferably a pyridin-2-yl group, a pyridin-3-yl group or a pyridin-4-yl group.

In another preferred embodiment, R₁ represents a heteroaryl group, preferably a pyridinyl group, which is substituted by from 1 to 2 groups selected from halogen or (C₁-C₆)alkoxy(C₁-C₆)alkoxy.

Preferably, R₁ represents a heteroaryl group, more preferably a pyridinyl group, which is substituted by from 1 to 2 groups selected from bromine, chlorine, or methoxyethoxy.

Advantageously, R₂ represents a hydrogen atom or a methyl group, preferably a hydrogen atom.

Preferably, the pair (R₁,R₂) together with the nitrogen atom to which they are attached forms an indolinyl group.

More preferably, the pair (R₁,R₂) together with the nitrogen atom to which they are attached forms an indolinyl group which is substituted by a halogen atom, preferably a chlorine atom.

Advantageously, R₁₆ represents a —O—R₃ group. In one embodiment, R₁₆ represents a —NR₁₇R₁₇′ group. In one preferred embodiment, R₁₆ represents a —NH₂ group, a —NH—CH₃ group, a —N(CH₃)₂ group, a —NH—SO₂—CF₃ group, or a —NH—SO₂—CH₃ group.

R₃ preferably represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)—(OR_{3A})₂, —W₁—SO₂—OR_{3A}, or —W₁—Cy₁, wherein:
W₁ represents a bond, a —CH₂— group, a —(CH₂)₂— group, a —(CH₂)₃— group, or a —CH(CH₃)— group,
R_{3A} and R_{3B} independently of one another, represent a hydrogen atom, a methyl group, an ethyl group, an isopropyl group, a tert-butyl group, a methoxymethyl group, a methoxyethyl group, or a cyclopentyl group,
or the pair (R_{3A},R_{3B}) together with the nitrogen atom to which they are attached forms a morpholinyl group,
Cy₁ represents an indanyl group or a 5-methyl-2-oxo-1,3-dioxol-4-yl group.

More preferably, R₃ represents a hydrogen atom, a linear or branched (C₁-C₆)alkyl group, a linear or branched halo(C₁-C₆)alkyl group, —(CH₂)₂—OR_{3A}, —CH(CH₃)—O—C(O)—R_{3A}, —CH₂—O—C(O)—R_{3A}, —(CH₂)₂—NR_{3A}R_{3B}, —CH₂—C(O)—NR_{3A}R_{3B}, —CH(CH₃)—O—C(O)—OR_{3A}, —CH₂—O—C(O)—OR_{3A}, —CH(CH₃)—O—C(O)—NR_{3A}R_{3B}, —CH₂—O—C(O)—NR_{3A}R_{3B}, —CH₂—O—P(O)—(OR_{3A})₂, —(CH₂)₃—SO₂—OR_{3A}, —Cy₁, or —CH₂—Cy₁.

Even more preferably, R₃ represents a hydrogen atom or a group selected from methyl, ethyl, isopropyl, 2,2,2-trifluoroethyl, methoxyethoxyethyl, methoxyethyl, N,N-dimethylaminoethyl, N,N-dimethylamidomethyl, indan-5-yl, (5-methyl-2-oxo-1,3-dioxol-4-yl)methyl, —CH(CH₃)—O—C(O)—CH₃, —CH(CH₃)—O—C(O)—CH₂CH₃, —CH(CH₃)—O—C(O)—CH₂—O—CH₃, —CH₂—O—C(O)—C(CH₃)₃, —CH(CH₃)—O—C(O)—O—CH₃, —CH(CH₃)—O—C(O)—O—CH(CH₃)₂, —CH(CH₃)—O—C(O)—O— cyclopentyl, —CH(CH₃)—O—C(O)—NH—(CH₂)₂O—CH₃, —CH₂—O—C(O)—O—CH₃, —CH(CH₃)—O—C(O)—N-morpholinyl, —CH(CH₃)—O—C(O)—N(CH₂CH₃)₂, —CH₂—O—C(O)—N(CH₂CH₃)₂, —CH₂—O—P(O)(O—C(CH₃)₃)₂, —CH₂—O—P(O)(OH)₂, or —(CH₂)₃—SO₂—OH.

Preferably, R₃ represents a hydrogen atom.

Advantageously, R₄ represents a hydrogen atom, a halogen atom, a linear or branched (C₁-C₆)alkyl group, a branched (C₁-C₆)alkyl group substituted by 2 linear (C₁-C₆)alkoxy groups, a linear or branched (C₂-C₆)alkenyl group, a linear or branched (C₂-C₆)alkenyloxy group, a linear or branched (C₁-C₆)alkoxy(C₁-C₆)alkyl group, a linear or branched (C₁-C₆)alkoxy(C₂-C₆)alkenyl group, a linear or branched (C₁-C₆)alkoxy(C₁-C₆)haloalkyl group, a —W₂—Cy₂ group, a —W₃-L-Cy₃ group, a —W₄—NR_{4A}R_{4B} group, or a —CO—NR_{4C}R_{4D} group, wherein
W₂ represents a bond, a linear or branched (C₁-C₄)alkylene group, a linear or branched (C₂-C₄)alkenylene group, or a linear or branched (C₂-C₄)alkynylene group,
W₃ represents a bond, a linear or branched (C₁-C₆)alkylene group, a linear or branched (C₂-C₈)alkynylene group, a linear or branched (C₁-C₆)alkoxylene group, a branched (C₁-C₄)hydroxyalkylene group, a linear or branched (C₁-C₄)haloalkylene, or
a —CH₂—CH(R_{4E})—CH₂— group,
L represents —O—, —S—, or —SO₂—,
Cy₂ represents an aryl group, a heteroaryl group, a cycloalkyl group, a cycloalkenyl group, or a heterocycloalkyl group,
Cy₃ represents an aryl group, a heteroaryl group, a heterocycloalkyl group, an arylalkyl group, or a heteroarylalkyl group.

Preferably, R₄ represents a hydrogen atom, a bromine atom, a iodine atom, a methyl group, an ethyl group, a propyl group, a 3-methoxy-2-(methoxymethyl)propyl, a prop-1-en-1-yl group, a (prop-2-en-1-yl)oxy group, a methoxypropyl group, an ethoxypropyl group, a 3-ethoxyprop-1-en-1yl group, a 2,2-difluoro-3-methoxypropyl group, a —W₂—Cy₂ group, a —W₃-L-Cy₃ group, a —W₄—NR_{4A}R_{4B} group, or a —CO—NR_{4C}R_{4D} group.

In one preferred embodiment, R₄ represents a —W₂-Cy₂ group.

Preferably, W₂ represents a bond, a linear or branched (C₁-C₄)alkylene group, a linear (C₂-C₄)alkenylene group, or a linear (C₂-C₄)alkynylene group.

More preferably, W₂ represents a bond, a —CH₂— group, a —(CH₂)₂— group, a —(CH₂)₃— group, a —(CH₂)₄— group, a —CH₂—CH(CH₃)— group, a —CH₂—CH(CH₃)—CH₂— group, a —CH═CH— group, a —CH═CH—CH₂— group, or a —C≡C— group.

Cy₂ preferably represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydroquinolinyl group, a tetrahydropyridinyl group, an azetidinyl group, a pyridinyl group, a pyrimidinyl group, a piperidinyl group, a morpholinyl group, a piperazinyl group, a cyclohexenyl group, a bicyclo[2.2.1]heptenyl group, a cyclopentenyl group, or a cyclohexyl group.

More preferably, Cy₂ represents an aryl group, a heteroaryl group, a cycloalkyl group, a cycloalkenyl group, or a heterocycloalkyl group which are substituted by 1, 2 or 3 groups selected from halogen, linear or branched (C₁-C₆)alkyl, linear or branched halo(C₁-C₆)alkyl, linear or branched (C₁-C₆)alkoxy, hydroxy, oxo, trifluoromethoxy, methoxyethoxy, —C(O)—OR′, phenyl, benzyl, (2,3,6-trifluorophenyl)methyl, pyridinyl, —CH₂-pyridinyl, —O-phenyl, —O— benzyl, —O—CH₂-cyclopropyl, —O-pyridinyl, —O—CH₂-pyridinyl, aryloxyalkyl, or heteroaryloxyalkyl, wherein R′ represents a linear or branched (C₁-C₆)alkyl group.

Even more preferably, Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydroquinolinyl group, a tetrahydropyridinyl group, an azetidinyl group, a pyridinyl group, a pyrimidinyl group, a piperidinyl group, a morpholinyl group, a piperazinyl group, a cyclohexenyl group, a bicyclo[2.2.1]heptenyl group, a cyclopentenyl group, or a cyclohexyl group which are substituted by 1, 2 or 3 groups selected from halogen, linear or branched (C₁-C₆)alkyl, linear or branched halo(C₁-C₆)alkyl, linear or branched (C₁-C₆)alkoxy, hydroxy, oxo, trifluoromethoxy, methoxyethoxy, —C(O)—OR′, phenyl, benzyl, (2,3,6-trifluorophenyl)methyl, pyridinyl, —CH₂-pyridinyl, —O-phenyl, —O-benzyl, —O—CH₂-cyclopropyl, —O-pyridinyl, —O—CH₂-pyridinyl, aryloxyalkyl, or heteroaryloxyalkyl, wherein R′ represents a linear or branched (C₁-C₆)alkyl group.

Advantageously, Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydroquinolinyl group, a tetrahydropyridinyl group, an azetidinyl group, a pyridinyl group, a pyrimidinyl group, a piperidinyl group, a morpholinyl group, a piperazinyl group, a cyclohexenyl group, a bicyclo[2.2.1]heptenyl group, a cyclopentenyl group, or a cyclohexyl group which are substituted by 1, 2 or 3 groups selected from chlorine, fluorine, methyl, —CF₃, —CH₂—CF₃, methoxy, ethoxy, hydroxy, oxo, trifluoromethoxy, methoxyethoxy, —CO—O—C(CH₃)₃, phenyl, benzyl, (2,3,6-trifluorophenyl)methyl, pyridinyl, —CH₂-pyridinyl, —O-phenyl, —O-benzyl, O—CH₂-cyclopropyl, —O-pyridinyl, —O—CH₂-pyridinyl, —CH₂—O-phenyl, —CH₂—O— pyridinyl, or —CH₂—O-thienopyridinyl.

Preferably, R₄ represents a —W₂-Cy₂ group wherein W₂ represents a bond and Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydropyridinyl group, an azetidinyl group, a cyclohexenyl group, a bicyclo[2.2.1]heptenyl group, or a cyclopentenyl group.

More preferably, R₄ represents a —W₂-Cy₂ group wherein W₂ represents a bond and Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydropyridinyl group, an azetidinyl group, a cyclohexenyl group, a bicyclo[2.2.1]heptenyl group, or a cyclopentenyl group which are substituted by 1, 2 or 3 groups selected from halogen, linear or branched (C₁-C₆)alkyl, linear or branched halo(C₁-C₆)alkyl, linear or branched (C₁-C₆)alkoxy, hydroxy, trifluoromethoxy, methoxyethoxy, phenyl, benzyl, (2,3,6-trifluorophenyl)methyl, pyridinyl, —CH₂-pyridinyl, —O-phenyl, —O-benzyl, —O—CH₂-cyclopropyl, —O-pyridinyl, —O—CH₂-pyridinyl, aryloxyalkyl, or heteroaryloxyalkyl.

Even more preferably, R₄ represents a —W₂-Cy₂ group wherein W₂ represents a linear or branched (C₁-C₄)alkylene group, a linear or branched (C₂-C₄)alkenylene group, or a linear or branched (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group.

Advantageously, R₄ represents a —W₂-Cy₂ group wherein W₂ represents a linear or branched (C₁-C₄)alkylene group, a linear (C₂-C₄)alkenylene group, or a linear (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group.

More advantageously, R₄ represents a —W₂-Cy₂ group wherein W₂ represents a linear or branched (C₁-C₄)alkylene group, a linear or branched (C₂-C₄)alkenylene group, or a linear or branched (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group which are substituted by 1, 2 or 3 groups selected from halogen, linear or branched (C₁-C₆)alkyl, linear or branched (C₁-C₆)alkoxy, oxo, —C(O)—OR′, or phenyl, wherein R′ represents a linear or branched (C₁-C₆)alkyl group.

In one preferred embodiment, R₄ represents a —W₂-Cy₂ group wherein W₂ represents a linear or branched (C₁-C₄)alkylene group, a linear (C₂-C₄)alkenylene group, or a linear (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group which are substituted by 1, 2 or 3 groups selected from halogen, linear or branched (C₁-C₆)alkyl, linear or branched (C₁-C₆)alkoxy, oxo, —C(O)—OR′, or phenyl, wherein R′ represents a linear or branched (C₁-C₆)alkyl group.

In another preferred embodiment, R₄ represents a —W₂-Cy₂ group wherein W₂ represents a linear or branched (C₁-C₄)alkylene group, a linear (C₂-C₄)alkenylene group, or a linear (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group which are substituted by 1, 2 or 3 groups selected from chlorine, methyl, methoxy, oxo, —CO—O—C(CH₃)₃, or phenyl.

In another preferred embodiment, R₄ represents a —W₃-L-Cy₃ group.

W₃ preferably represents a bond, a linear or branched (C₁-C₆)alkylene group, a linear or branched (C₂-C₈)alkynylene group, a linear or branched (C₁-C₆)alkoxylene group, a branched (C₁-C₄)hydroxyalkylene group, a linear or branched (C₁-C₄)haloalkylene group, or a —CH₂—CH(R_{4E})—CH₂— group.

More preferably, W₃ represents a bond, a —CH₂— group, a —(CH₂)₂— group, a —(CH₂)₃— group, a —(CH₂)₄— group, a —CH₂—CH(CH₃)—CH₂— group, a —CH₂—CH(CH₂—CH₃)—CH₂— group, a —CH₂—CH[CH(CH₃)₂]—CH₂— group, a —CH₂—CH(CH₃)—CH(CH₃)— group, a —CH₂—C(CH₃)₂—CH₂— group, a —O—(CH₂)₃— group, a —CH₂—CH(CH₂—OH)—CH₂— group, a —CH₂—CH(CH₂—OCH₃)—CH₂— group, a —CH₂—CF₂—CH₂— group, a —CH₂—CHF—CH₂— group, a —CH₂—CH(CH₂F)—CH₂— group, a —CH₂—CH(CHF₂)—CH₂— group, a —CH₂—CF(CH₃)—CH₂— group, a —CH₂—CH(C≡C—CH₂—CH₂—CH₃)—CH₂— group, a —C≡C—CH₂— group, a —C≡C—CH₂—CH₂— group, or a —CH₂—CH(R_{4E})—CH₂— group.

Even more preferably, W₃ represents a —CH₂—CH(CH₃)—CH₂— group.

Preferably, L represents —O—.

In a preferred embodiment, L represents —S— or —SO₂—.

Cy₃ preferably represents a phenyl group, a thienopyridinyl group, a pyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, a benzothiazolyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a cycloheptapyridinyl group, a pyranopyridinyl group, a tetrahydronaphthyridinyl group, a tetrahydro-5,8-ethanoquinolinyl group, a dihydroquinolinyl group, a benzyl group, a —(CH₂)₂-phenyl group, a —(CH₂)₃-phenyl group, or a —(CH₂)₃-pyridinyl group.

Advantageously, Cy₃ represents an aryl group, a heteroaryl group, a cycloalkyl group, a heterocycloalkyl group, an arylalkyl group, or a heteroarylalkyl group which are substituted by 1, 2 or 3 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, —NR′R″, cyclopropyl, 2,2-dimethylcyclopropyl, a linear or branched (C₁-C₆)hydroxyalkyl, hydroxy, oxo, or difluoromethoxy, wherein R′ and R″ independently of one another represent a hydrogen atom or a linear or branched (C₁-C₆)alkyl group.

More preferably, Cy₃ represents a phenyl group, a thienopyridinyl group, a pyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, a benzothiazolyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a cycloheptapyridinyl group, a pyranopyridinyl group, a tetrahydronaphthyridinyl group, a tetrahydro-5,8-ethanoquinolinyl group, a dihydroquinolinyl group, a benzyl group, a —(CH₂)₂-phenyl group, a —(CH₂)₃-phenyl group, or a —(CH₂)₃-pyridinyl group which are substituted by 1, 2 or 3 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, —NR′R″, cyclopropyl, 2,2-dimethylcyclopropyl, a linear or branched (C₁-C₆)hydroxyalkyl, hydroxy, oxo, or difluoromethoxy, wherein R′ and R″ independently of one another represent a hydrogen atom or a linear or branched (C₁-C₆)alkyl group.

Even more preferably, Cy₃ represents a phenyl group, a thienopyridinyl group, a pyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, a benzothiazolyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a cycloheptapyridinyl group, a pyranopyridinyl group, a tetrahydronaphthyridinyl group, a tetrahydro-5,8-ethanoquinolinyl group, a dihydroquinolinyl group, a benzyl group, a —(CH₂)₂-phenyl group, a —(CH₂)₃-phenyl group, or a —(CH₂)₃-pyridinyl group which are substituted by 1, 2 or 3 groups selected from fluorine, chlorine, methyl, ethyl, isopropyl, —CHF₂, —CF₃, —CH₂CF₃, ═CHF, methoxy, —NH₂, —NH(CH₃), cyclopropyl, 2,2-dimethylcyclopropyl, hydroxymethyl, hydroxy, oxo, or difluoromethoxy.

Preferably, R₄ represents a —W₃-L-Cy₃ group wherein W₃ represents a bond, L represents —O— and Cy₃ represents an arylalkyl group or a heteroarylalkyl group.

In a preferred embodiment, R₄ represents a —W₃-L-Cy₃ group wherein W₃ represents a bond, L represents —O— and Cy₃ represents a —(CH₂)₃-phenyl group or a —(CH₂)₃-pyridinyl group.

In another preferred embodiment, R₄ represents a —W₃-L-Cy₃ group wherein W₃ represents a linear or branched (C₁-C₆)alkylene group, L represents —O— and Cy₃ represents an aryl group, a heteroaryl group, a heterocycloalkyl group, or an arylalkyl group.

Advantageously, R₄ represents a —W₃-L-Cy₃ group wherein W₃ represents a linear or branched (C₁-C₆)alkylene group, L represents —O— and Cy₃ represents a phenyl group, a pyridinyl group, a thienopyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a benzothiazolyl group, a cycloheptapyridinyl group, a pyranopyridinyl group, a tetrahydronaphthyridinyl group, a tetrahydro-5,8-ethanoquinolinyl group, a dihydroquinolinyl group a benzyl group, or a —(CH₂)₂-phenyl group.

In another preferred embodiment, R₄ represents a —W₃-L-Cy₃ group wherein W₃ represents a —CH₂—CH(CH₃)—CH₂— group, L represents —O— and Cy₃ represents a tetrahydroquinolinyl group. Preferably, R₄ represents a —W₃-L-Cy₃ group wherein W₃ represents a —CH₂—CH(CH₃)—CH₂— group, L represents —O— and Cy₃ represents a tetrahydroquinolinyl group which is substituted by 1 or 2 groups selected from halogen, preferably a fluorine atom, linear or branched (C₁-C₆)alkyl, preferably a methyl group, or hydroxy.

Advantageously, R₄ represents

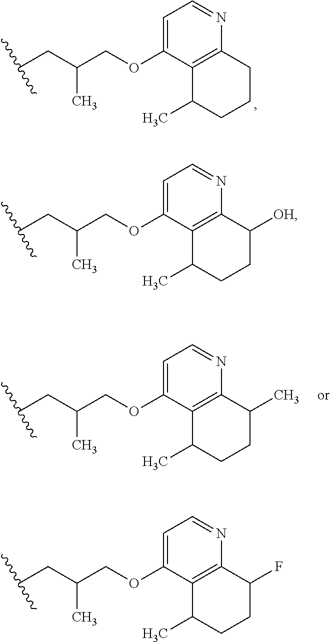 [SMILES: *CC(C)COc1ccnc2c1C(C)CCC2 ; *CC(C)COc1ccnc2c1C(C)CCC2O ; *CC(C)COc1ccnc2c1C(C)CCC2C ; *CC(C)COc1ccnc2c1C(C)CCC2F]

wherein the wavy line indicates the covalent attachment site to the spirocyclohexane scaffold.

Preferably, R₄ represents

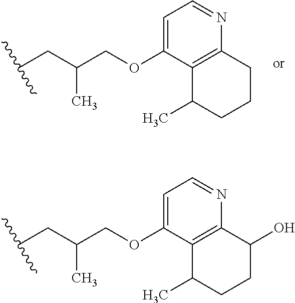 [SMILES: *CC(C)COc1ccnc2c1C(C)CCC2 ; *CC(C)COc1ccnc2c1C(C)CCC2O]

wherein the wavy line indicates the covalent attachment site to the spirocyclohexane scaffold.

More advantageously, R₄ represents

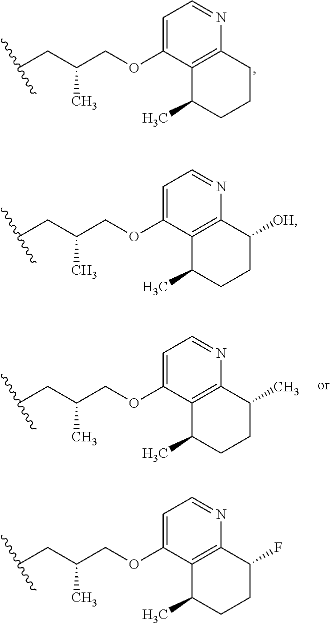 [SMILES: *C[C@@H](C)COc1ccnc2c1[C@H](C)CCC2 ; *C[C@@H](C)COc1ccnc2c1[C@H](C)CC[C@H]2O ; *C[C@@H](C)COc1ccnc2c1[C@H](C)CC[C@H]2C ; *C[C@@H](C)COc1ccnc2c1[C@H](C)CC[C@H]2F]

wherein the wavy line indicates the covalent attachment site to the spirocyclohexane scaffold.

Preferably, R₄ represents

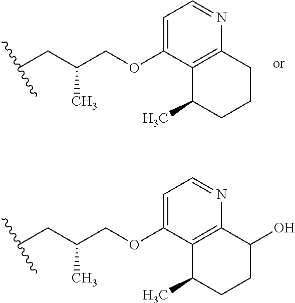 [SMILES: *C[C@@H](C)COc1ccnc2c1[C@H](C)CCC2 ; *C[C@@H](C)COc1ccnc2c1[C@H](C)CCC2O]

wherein the wavy line indicates the covalent attachment site to the spirocyclohexane scaffold.

Even more advantageously, R₄ represents

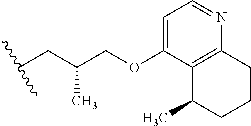 [SMILES: *C[C@@H](C)COc1ccnc2c1[C@H](C)CCC2]

wherein the wavy line indicates the covalent attachment site to the spirocyclohexane scaffold.

R₄ preferably represents a —W₃-L-Cy₃ group wherein W₃ represents a linear or branched (C₂-C₈)alkynylene group, L represents —O— and Cy₃ represents a phenyl group, or a thienopyridinyl group.

In another preferred embodiment, R₄ represents a —W₃-L-Cy₃ group wherein W₃ represents a linear or branched (C₁-C₄)alkoxylene group, L represents —O— and Cy₃ represents a pyridinyl group, or a cyclopentapyridinyl group.

In another preferred embodiment, R₄ represents a —W₃-L-Cy₃ group wherein W₃ represents a branched (C₁-C₄)hydroxyalkylene group, L represents —O— and Cy₃ represents a thienopyridinyl group, or a cyclopentapyridinyl group.

In another preferred embodiment, R₄ represents a —W₃-L-Cy₃ group wherein W₃ represents a linear or branched (C₁-C₄)haloalkylene group, L represents —O— and Cy₃ represents a cyclopentapyridinyl group, a tetrahydroquinolinyl group, or a thienopyridinyl group.

In another preferred embodiment, R₄ represents a —W₃-L-Cy₃ group wherein W₃ represents a —CH₂—CH(R_{4E})—CH₂— group, L represents —O— and Cy₃ represents a phenyl group, a thienopyridinyl group, a cyclopentapyridinyl group, a pyridinyl group, or a tetrahydroquinolinyl group.

Preferably, R_{4E} represents —Cy₄.

More preferably, R_{4E} represents —CH₂—O—Cy₄.

Preferably, Cy₄ represents a phenyl group, a benzyl group, a pyridinyl group, a thienopyridinyl group, a cyclopentapyridinyl group, or a —(CH₂)₂-morpholinyl group.

Advantageously, R_{4E} represents —Cy₄ wherein Cy₄ represents a phenyl group, a benzyl group, or a pyridinyl group.

Advantageously, R_{4E} represents —CH₂—O—Cy₄ wherein Cy₄ represents a thienopyridinyl group, a cyclopentapyridinyl group, or a —(CH₂)₂-morpholinyl group.

Preferably, R₄ represents a —W₄—NR_{4A}R_{4B} group.

Preferably, W₄ represents a —CH₂— group, a —(CH₂)₂— group, or a —CH₂—CH(CH₃)—CH₂— group.

Preferably, R_{4A} and R_{4B} independently of one another represent a hydrogen atom, a methyl group, a phenyl group, a pyridinyl group, a thienopyridinyl group, a tetrahydroquinolinyl group, or a benzyl group.

Preferably, R_{4A} represents a phenyl group, a pyridinyl group, a thienopyridinyl group, a tetrahydroquinolinyl group, or a benzyl group.

Preferably, R_{4B} represents a hydrogen atom, or a methyl group.

Preferably, R₄ represents a —CO—NR_{4C}R_{4D} group.

Preferably, R_{4C} and R_{4D} independently of one another represent a hydrogen atom, a methyl group, a benzyl group, a —(CH₂)₂-phenyl group, or a —(CH₂)₂-pyridinyl group.

Preferably, R_{4C} represents a benzyl group, a —(CH₂)₂-phenyl group, or a —(CH₂)₂-pyridinyl group.

Preferably, R_{4D} represents a hydrogen atom, or a methyl group.

Preferably, R₅ represents a hydrogen atom or a methyl group, more preferably a hydrogen atom.

Preferably, the pair (R₄,R₅) represents an oxo group, or a cyclopentylidene group.

More preferably, the pair (R₄,R₅) together with carbon atoms to which they are attached forms a cyclopentyl ring.

Preferably, R₆ represents a phenyl group, a —SO₂-phenyl group, or a —W₅—O—Cy₅ group.

Preferably, W₅ represents a —(CH₂)₃— group, or a —CH₂—CH(CH₃)—CH₂— group, more preferably a —CH₂—CH(CH₃)—CH₂— group.

Preferably, Cy₅ represents a phenyl group, a thienopyridinyl group, a pyridinyl group, an indolyl group, or a tetrahydroquinolinyl group, more preferably a tetrahydroquinolinyl group, even more preferably, a tetrahydroquinolinyl group which is substituted by 1 or 2 groups selected from linear or branched (C₁-C₆)alkyl, preferably a methyl group.

More preferably, R₆ represents

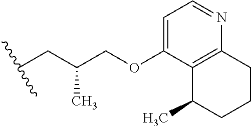 [SMILES: *C[C@@H](C)COc1ccnc2c1[C@H](C)CCC2]

wherein the wavy line indicates the covalent attachment site to the spirocyclohexane scaffold.

Preferably, R₇ represents a hydrogen atom, a methyl group, an ethyl group, a benzyl group, or a formyl group.

Preferably, R₈ represents a hydrogen atom, a methyl group, an ethyl group, or an isopropyl group. More preferably, R₈ represents a hydrogen atom.

Preferably, the pair (R₄,R₈) together with carbon atoms to which they are attached forms a cyclopropyl ring or a phenyl ring.

Preferably, R₉ represents a hydrogen atom.

Preferably, R₁₀ represents a hydrogen atom, a chlorine atom, a fluorine atom, a bromine atom, or a methyl group. More preferably, R₁₀ represents a hydrogen atom.

Preferably, the pair (R₇,R₁₀) together with the nitrogen atom to which they are attached forms a non-aromatic ring composed of 6 ring members.

Preferably, R₁ represents a hydrogen atom, a chlorine atom, a fluorine atom, a bromine atom, a methyl group, or a methoxy group.

More preferably, R₁ represents a hydrogen atom, a chlorine atom, a fluorine atom, or a methyl group. Even more preferably, Rn represents a hydrogen atom.

R₁₂ preferably represents a hydrogen atom, a fluorine atom, a bromine atom, a iodine atom, a chlorine atom, a methyl group, an ethyl group, a prop-1-enyl group, a —C≡CH group, a methoxy group, an ethoxy group, a propoxy group, an isopropoxy group, an isobutyloxy group, a 2-methoxypropan-2-yl group, a prop-2-en-1-yloxy group, a 2,2,2-trifluoroethoxy group, a methoxymethyl group, a methoxyethoxy group, a methoxypropoxy group, a hydroxy group, a hydroxymethyl group, a 1-hydroxyethyl group, an acetyl group, a formyl group, a —CH₂—O— tetrahydrofuranyl group, —Cy₆, or —O—Cy₇.

More preferably, R₁₂ represents a hydrogen atom, a methyl group, a methoxy group, an ethoxy group, an isopropoxy group, a 2-methoxypropan-2-yl group, a methoxymethyl group, a methoxyethoxy group or a 1-hydroxyethyl group.

Even more preferably, R₁₂ represents —Cy₆ which is selected from a phenyl group, a cyclopropyl group, a thienyl group, a pyrrolyl group, a —(CH₂)₂-phenyl group, a —CH(CH₃)-phenyl group, a —CH═CH-phenyl group, or a —CH═CH—CH₂-phenyl group.

Advantageously, R₁₂ represents —Cy₆ which is selected from a phenyl group, a cyclopropyl group, a thienyl group, a pyrrolyl group, a —(CH₂)₂-phenyl group, a —CH(CH₃)-phenyl group, a —CH═CH-phenyl group, or a —CH═CH—CH₂-phenyl group which are substituted by 1, 2 or 3 groups selected from linear or branched (C₁-C₆)alkyl, linear or branched (C₁-C₆)alkoxy, linear or branched (C₁-C₆)alkoxy(C₁-C₆)alkyl, or hydroxy.

More advantageously, R₁₂ represents —Cy₆ which is selected from a phenyl group, a cyclopropyl group, a thienyl group, a pyrrolyl group, a —(CH₂)₂-phenyl group, a —CH(CH₃)-phenyl group, a —CH═CH-phenyl group, or a —CH═CH—CH₂-phenyl group which are substituted by 1, 2 or 3 groups selected from methyl, methoxy, methoxymethyl, or hydroxy.

In a preferred embodiment, R₁₂ represents —O—Cy₇ wherein Cy₇ represents a phenyl group, a cyclopentyl group, or a —CH₂-cyclopropyl group.

Preferably, the pair (R₁₁,R₁₂) together with the carbon atoms to which they are attached forms a non-aromatic ring composed of from 5 to 8 ring members, which may contain 1 or 2 oxygen atoms, wherein said ring may be substituted by R₁₈ and R₁₈′.

More preferably, the pair (R₁₁,R₁₂) together with the carbon atoms to which they are attached forms a non-aromatic ring as follows:

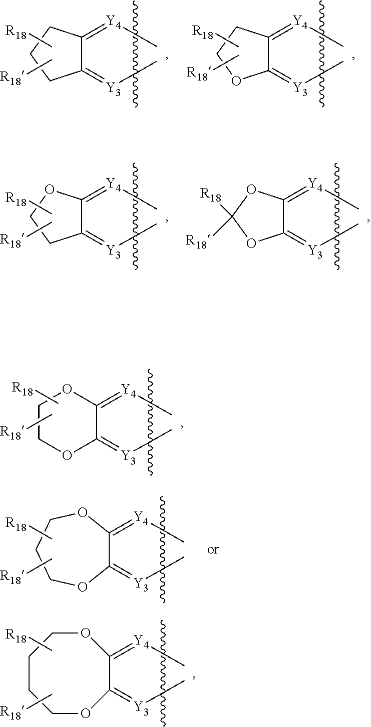

wherein R₁₈ and R₁₈′ are as defined for Formula (I).

Even more preferably, the pair (R₁₁,R₁₂) together with the carbon atoms to which they are attached forms a non-aromatic ring as follows:

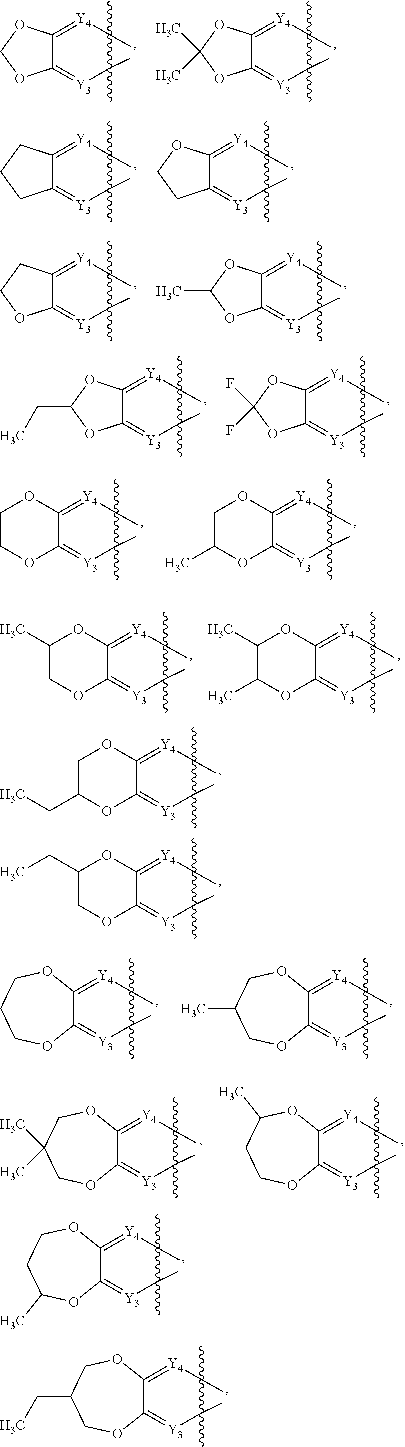

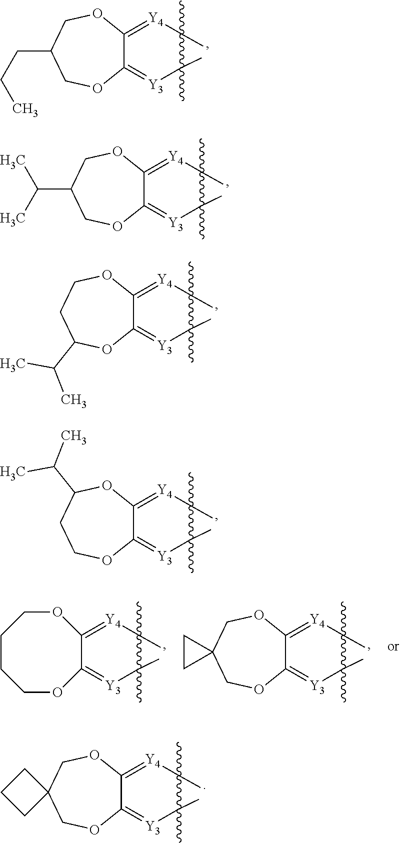

Advantageously, the pair (R₁₁,R₁₂) together with the carbon atoms to which they are attached forms a non-aromatic ring as follows:

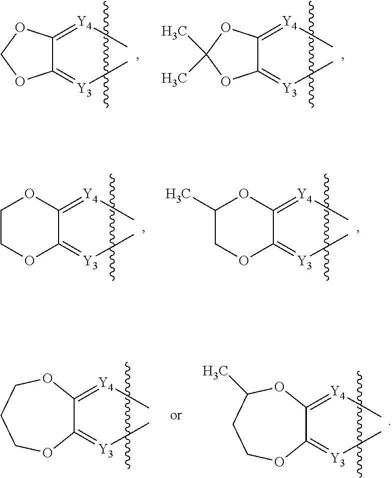

More advantageously, the pair (R₁₁,R₁₂) together with the carbon atoms to which they are attached forms a non-aromatic ring as follows:

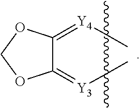

Preferably, R₁₃ represents a hydrogen atom, a fluorine atom, a bromine atom, or a methyl group. More preferably, R₁₃ represents a hydrogen atom.

Preferably, R₁₄ and R₁₅ represent a hydrogen atom.

Preferably, R₁₇ and R₁₇′ independently of one another, represent a hydrogen atom, a methyl group, a —SO₂—CF₃ group, or a —SO₂—CH₃ group. In one embodiment, R₁₇ represents a hydrogen atom, a methyl group, a —SO₂—CF₃ group, or a —SO₂—CH₃ group. In one embodiment, R₁₇′ represents a hydrogen atom or a methyl group.

Preferably, R₁₈ and R₁₈′ independently of one another, represent a hydrogen atom, a fluorine atom, a methyl group, an ethyl group, a n-propyl group or an isopropyl group. In a preferred embodiment, R₁₈ represents a hydrogen atom, a methyl group, an ethyl group, a n-propyl group or an isopropyl group and R₁₈′ represents a hydrogen atom. More preferably, R₁₈ and R₁₈′ represent both a hydrogen atom. In another embodiment, R₁₈ and R₁₈′ represent both a fluorine atom. In another embodiment, R₁₈ and R₁₈′ represent both a methyl group. In another embodiment, the pair (R₁₈,R₁₈′) together with the carbon atoms to which they are attached forms a cyclopropyl ring or a cyclobutyl ring.

Preferred compounds according to the invention are:
(1s,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid;
(1s,4S)-4-(3-chloro-4-fluoroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid;
(1s,4S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid;
(1s,4S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid;
(1r,2′S,4S)-4-(3-chloro-2-methylanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid;
(1r,2′S,4S)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-[(propan-2-yl)oxy]-2′,3′-dihydrospiro[cyclohexane-1,1′-indene]-4-carboxylic acid;
(1r,2′S,4S)-4-(3-chloroanilino)-6′-(2-methoxyethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-5′-chloro-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,4S,8'S)-4-(3-chloroanilino)-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid;
(1r,4S,7'S)-4-(3-chloroanilino)-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,4S,6'S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-2-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-2-methylanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloroanilino)-2′,2′-dimethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,2′S,4S)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′R,4R)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(2-methoxypropan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,4S,6'S)-4-(3-chloroanilino)-6′-[(2R)-3-{[(5R,8R)-8-hydroxy-5-methyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid;
(1r,4S,7'S)-4-(3-chloroanilino)-7′-[(2R)-3-{[(5R,8R)-8-hydroxy-5-methyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylic acid;
(1r,3'S,4S,7'S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,3′R,4S,7'S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,4S,4'S,8'S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid;
(1r,4S,4′R,8'S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid.

Another aspect of the invention concerns a compound of Formula (IIIA):

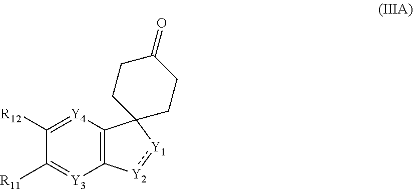

wherein R₁₁, R₁₂, Y₁, Y₂, Y₃, Y₄ and [symbol] are as defined in Formula (I).

Advantageously, compound of Formula (IIIA) can be used as synthesis intermediate for the preparation of compounds of Formula (I). Preferably, compound of Formula (IIIA) is 6′-bromo-2′H-spiro[cyclohexane-1,5′-indeno[5,6-d][1,3]dioxol]-4-one.

Another aspect of the invention concerns a compound of Formula (VA):

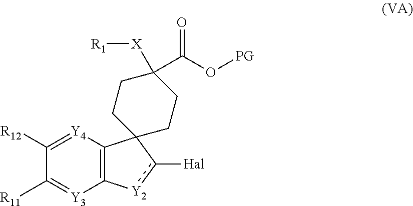

wherein R₁, R₁₁, R₁₂, X, Y₂, Y₃, Y₄ and [symbol] are as defined in E1, and Hal represents a halogen atom and PG represents a protecting group of the carboxylic acid function.

Advantageously, compound of Formula (VA) can be used as synthesis intermediate for the preparation of compounds of Formula (I). Preferably, compound of Formula (VA) is:
methyl (1s,4s)-2′-bromo-4-(3-chloroanilino)spiro[cyclohexane-1,1′-indene]-4-carboxylate;
methyl (1s,4s)-6′-bromo-4-(3-chloroanilino)-2′H-spiro[cyclohexane-1,5′-indeno[5,6-d][1,3]dioxole]-4-carboxylate.

Described below are a number of embodiments of the invention, where for convenience E1 is identical to the first aspect of the invention hereinabove. Further enumerated embodiments (E) of the invention are described herein. It will be recognized that features specified in each embodiment may be combined with other specified features to provide further embodiments of the present invention.
E2. Compounds according to E1 wherein X represents —O— or —N(R₂)—.
E3. Compounds according to E1 wherein X represents —N(R₂)—.
E4. Compounds according to E1 wherein X represents —NH—.
E5. Compounds according to E1 wherein [symbol] represents a single bond.
E6. Compounds according to E1 wherein, when [symbol] represents a single bond, independently of one another, Y₁ represents —C(R₄)(R₅)— or —N(R₆)— and Y₂ represents —N(R₇)—, —C(R₈)(R₉)—, or —C(R₈)(R₉)—C(R₁₄)(R₁₅)—.
E7. Compounds according to E1 wherein Y₁ represents —C(R₄)(R₅)—.
E8. Compounds according to E1 wherein Y₁ represents —N(R₆)—.
E9. Compounds according to E1 wherein Y₂ represents —N(R₇)—.
E10. Compounds according to E1 wherein Y₂ represents —C(R₈)(R₉)—.
E11. Compounds according to E1 wherein Y₂ represents —C(R₈)(R₉)—C(R₁₄)(R₁₅)—.
E12. Compounds according to E1 wherein Y₁ represents —C(R₄)(R₅)— and Y₂ represents —C(R₈)(R₉)—.
E13. Compounds according to E1 wherein Y₁ represents —C(R₄)(R₅)— and Y₂ represents —N(R₇)—.
E14. Compounds according to E1 wherein Y₁ represents —N(R₆)— and Y₂ represents —C(R₈)(R₉)—.
E15. Compounds according to E1 wherein Y₁ represents —C(R₄)(R₅)— and Y₂ represents —C(R₈)(R₉)—C(R₁₄)(R₁₅)—.
E16. Compounds according to E1 wherein, when [symbol] represents a double bond, independently of one another, Y₁ represents —C(R₄)— and Y₂ represents —N═ or —C(R₈)—.
E17. Compounds according to E1 wherein Y₁-Y₂ represents —C(R₄)═N—.
E18. Compounds according to E1 wherein Y₁-Y₂ represents —C(R₄)═C(R₈)—.
E19. Compounds according to E1 wherein Y₃ represents —C(R₁₀)—.
E20. Compounds according to E1 wherein Y₄ represents —C(R₁₃)—.
E21. Compounds according to E1 wherein Y₃ represents —C(R₁₀)— and Y₄ represents —C(R₁₃)—.
E22. Compounds according to E1 wherein an advantageous possibility consists of compounds of Formula (I-a):

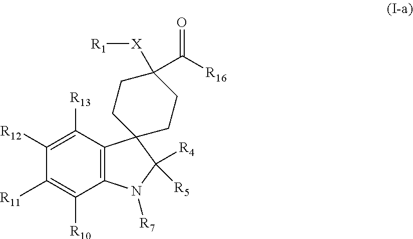

wherein R₁, R₄, R₅, R₇, R₁₀, R₁₁, R₁₂, R₁₃, R₁₆ and X are as defined for Formula (I).
E23. Compounds according to E1 wherein an advantageous possibility consists of compounds of Formula (I-b):

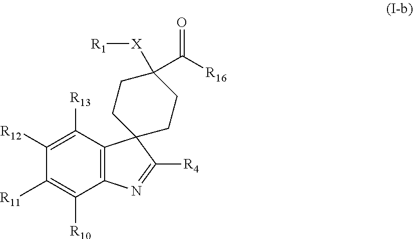

wherein R₁, R₄, R₁₀, R₁₁, R₁₂, R₁₃, R₁₆ and X are as defined for Formula (I).
E24. Compounds according to E1 wherein an advantageous possibility consists of compounds of Formula (I-c):

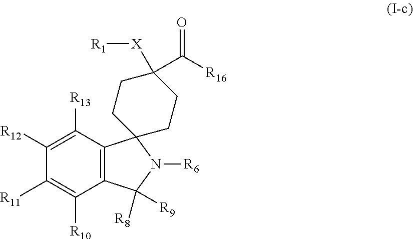

wherein R₁, R₆, R₈, R₉, R₁₀, R₁₁, R₁₂, R₁₃, R₁₆ and X are as defined for Formula (I).
E25. Compounds according to E1 wherein an advantageous possibility consists of compounds of Formula (I-d):

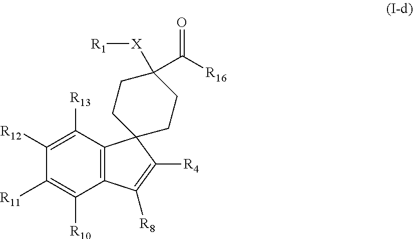

wherein R₁, R₄, R₈, R₁₀, R₁₁, R₁₂, R₁₃, R₁₆ and X are as defined for Formula (I).
E26. Compounds according to E1 wherein an advantageous possibility consists of compounds of Formula (I-e):

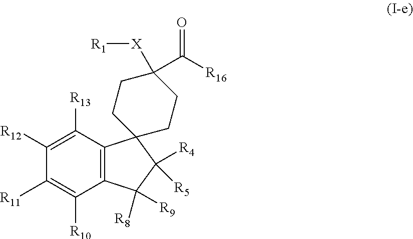

wherein R₁, R₄, R₅, R₈, R₉, R₁₀, R₁₁, R₁₂, R₁₃, R₁₆ and X are as defined for Formula (I).
E27. Compounds according to E1 wherein an advantageous possibility consists of compounds of Formula (I-f):

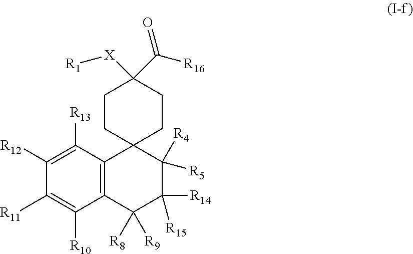

wherein R₁, R₄, R₅, R₈, R₉, R₁₀, R₁₁, R₁₂, R₁₃, R₁₄, R₁₅, R₁₆ and X are as defined for Formula (I).
E28. Compounds according to E1 wherein an advantageous possibility consists of compounds of Formula (I-g):

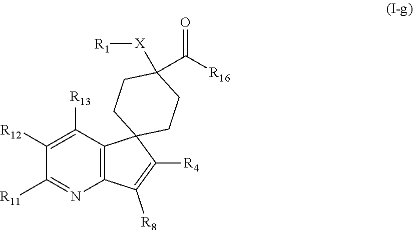

wherein R₁, R₄, R₈, R₁₁, R₁₂, R₁₃, R₁₆ and X are as defined for Formula (I).
E29. Compounds according to E1 wherein an advantageous possibility consists of compounds of Formula (I-h):

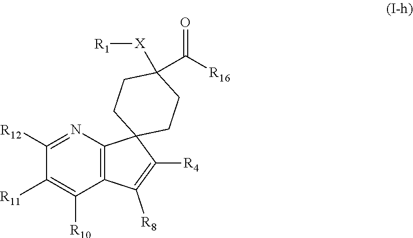

wherein R₁, R₄, R₈, R₉, R₁₀, R₁₁, R₁₂, R₁₆ and X are as defined for Formula (I).
E30. Compounds according to E1 wherein R₁ represents an aryl group, preferably a phenyl group.
E31. Compounds according to E1 wherein R₁ represents an aryl group, preferably a phenyl group, which is substituted by from 1 to 3 groups selected from halogen, linear or branched (C₁-C₆)alkyl, linear or branched halo(C₁-C₆)alkyl, linear or branched (C₁-C₆)alkoxy, cyano, or hydroxy.
E32. Compounds according to E1 wherein R₁ represents an aryl group, preferably a phenyl group, which is substituted by from 1 to 3 groups selected from fluorine, chlorine, bromine, methyl, difluoromethyl, trifluoromethyl, methoxy, cyano, or hydroxy.
E33. Compounds according to E1 wherein R₁ represents a 3-chloro-phenyl group, a 3-chloro-4-fluoro-phenyl group, a 3-chloro-2-fluoro-phenyl group, or a 3-chloro-2-methyl-phenyl group.
E34. Compounds according to E1 wherein R₁ represents a 3-chloro-phenyl group.
E35. Compounds according to E1 wherein R₁ represents a deuterated aryl group, preferably a deuterated phenyl group.
E36. Compounds according to E1 wherein R₁ represents a heteroaryl group, preferably a pyridinyl group, more preferably a pyridin-2-yl group, a pyridin-3-yl group or a pyridin-4-yl group.
E37. Compounds according to E1 wherein R₁ represents a heteroaryl group, preferably a pyridinyl group, which is substituted by from 1 to 2 groups selected from halogen or (C₁-C₆)alkoxy(C₁-C₆)alkoxy.
E38. Compounds according to E1 wherein R₁ represents a heteroaryl group, preferably a pyridinyl group, which is substituted by from 1 to 2 groups selected from bromine, chlorine, or methoxyethoxy.
E39. Compounds according to E1 wherein R₂ represents a hydrogen atom or a methyl group, preferably a hydrogen atom.
E40. Compounds according to E1 wherein the pair (R₁,R₂) together with the nitrogen atom to which they are attached forms an indolinyl group.
E41. Compounds according to E1 wherein the pair (R₁,R₂) together with the nitrogen atom to which they are attached forms an indolinyl group which is substituted by a halogen atom, preferably a chlorine atom.
E42. Compounds according to E1 wherein R₃ represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)—(OR_{3A})₂, —W₁—SO₂—OR_{3A}, or —W₁—Cy₁, wherein:
   W₁ represents a bond, a —CH₂— group, a —(CH₂)₂— group, a —(CH₂)₃— group, or a —CH(CH₃)— group,
   R_{3A} and R_{3B} independently of one another, represent a hydrogen atom, a methyl group, an ethyl group, an isopropyl group, a tert-butyl group, a methoxymethyl group, a methoxyethyl group, or a cyclopentyl group, or the pair (R_{3A},R_{3B}) together with the nitrogen atom to which they are attached forms a morpholinyl group,
   Cy₁ represents an indanyl group or a 5-methyl-2-oxo-1,3-dioxol-4-yl group.
E43. Compounds according to E1 wherein R₃ represents a hydrogen atom, a linear or branched (C₁-C₆)alkyl group, a linear or branched halo(C₁-C₆)alkyl group, —(CH₂)₂—OR_{3A}, —CH(CH₃)—O—C(O)—R_{3A}, —CH₂—O—C(O)—R_{3A}, —(CH₂)₂—NR_{3A}R_{3B}, —CH₂—C(O)—NR_{3A}R_{3B}, —CH(CH₃)—O—C(O)—OR_{3A}, —CH₂—O—C(O)—OR_{3A}, —CH(CH₃)—O—C(O)—NR_{3A}R_{3B}, —CH₂—O—C(O)—NR_{3A}R_{3B}, —CH₂—O—P(O)—(OR_{3A})₂, —(CH₂)₃—SO₂—OR_{3A}, —Cy₁, or —CH₂—Cy₁.
E44. Compounds according to E1 wherein R₃ represents a hydrogen atom or a group selected from methyl, ethyl, isopropyl, 2,2,2-trifluoroethyl, methoxyethoxyethyl, methoxyethyl, N,N-dimethylaminoethyl, N,N-dimethylamidomethyl, indan-5-yl, (5-methyl-2-oxo-1,3-dioxol-4-yl)methyl, —CH(CH₃)—O—C(O)—CH₃, —CH(CH₃)—O—C(O)—CH₂CH₃, —CH(CH₃)—O—C(O)—CH₂—O—CH₃, —CH₂—O—C(O)—C(CH₃)₃, —CH(CH₃)—O—C(O)—O—CH₃, —CH(CH₃)—O—C(O)—O—CH(CH₃)₂, —CH(CH₃)—O—C(O)—O-cyclopentyl, —CH(CH₃)—O—C(O)—NH—(CH₂)₂O—CH₃, —CH₂—O—C(O)—O—CH₃, —CH(CH₃)—O—C(O)—N-morpholinyl, —CH(CH₃)—O—C(O)—N(CH₂CH₃)₂, —CH₂—O—C(O)—N(CH₂CH₃)₂, —CH₂—O—P(O)(O—C(CH₃)₃)₂, —CH₂—O—P(O)(OH)₂, or —(CH₂)₃—SO₂—OH.
E45. Compounds according to E1 wherein R₃ represents a hydrogen atom.
E46. Compounds according to E1 wherein R₄ represents a hydrogen atom, a halogen atom, a linear or branched (C₁-C₆)alkyl group, a branched (C₁-C₆)alkyl group substituted by 2 linear (C₁-C₆)alkoxy groups, a linear or branched (C₂-C₆)alkenyl group, a linear or branched (C₂-C₆)alkenyloxy group, a linear or branched (C₁-C₆)alkoxy(C₁-C₆)alkyl group, a linear or branched (C₁-C₆)alkoxy(C₂-C₆)alkenyl group, a linear or branched (C₁-C₆)alkoxy(C₁-C₆)haloalkyl group, a —W₂-Cy₂ group, a —W₃-L-Cy₃ group, a —W₄—NR_{4A}R_{4B} group, or a —CO—NR_{4C}R_{4D} group, wherein
   W₂ represents a bond, a linear or branched (C₁-C₄)alkylene group, a linear or branched (C₂-C₄)alkenylene group, or a linear or branched (C₂-C₄)alkynylene group,
   W₃ represents a bond, a linear or branched (C₁-C₆)alkylene group, a linear or branched (C₂-C₈)alkynylene group, a linear or branched (C₁-C₆)alkoxylene group, a branched (C₁-C₄)hydroxyalkylene group, a linear or branched (C₁-C₄)haloalkylene, or a —CH₂—CH(R_{4E})—CH₂— group,
   L represents —O—, —S—, or —SO₂—,
   Cy₂ represents an aryl group, a heteroaryl group, a cycloalkyl group, a cycloalkenyl group, or a heterocycloalkyl group,
   Cy₃ represents an aryl group, a heteroaryl group, a heterocycloalkyl group, an arylalkyl group, or a heteroarylalkyl group.
E47. Compounds according to E1 wherein R₄ represents a hydrogen atom, a bromine atom, a iodine atom, a methyl group, an ethyl group, a propyl group, a 3-methoxy-2-(methoxymethyl)propyl, a prop-1-en-1-yl group, a (prop-2-en-1-yl)oxy group, a methoxypropyl group, an ethoxypropryl group, a 3-ethoxyprop-1-en-1yl group, a 2,2-difluoro-3-methoxypropyl group, a —W₂-Cy₂ group, a —W₃-L-Cy₃ group, a —W₄—NR_{4A}R_{4B} group, or a —CO—NR_{4C}R_{4D} group.
E48. Compounds according to E1 wherein R₄ represents a —W₂-Cy₂ group.
E49. Compounds according to E1 wherein W₂ represents a bond, a linear or branched (C₁-C₄)alkylene group, a linear (C₂-C₄)alkenylene group, or a linear (C₂-C₄)alkynylene group.
E50. Compounds according to E1 wherein W₂ represents a bond, a —CH₂— group, a —(CH₂)₂— group, a —(CH₂)₃— group, a —(CH₂)₄— group, a —CH₂—CH(CH₃)— group, a —CH₂—CH(CH₃)—CH₂— group, a —CH═CH— group, a —CH═CH—CH₂— group, or a —C≡C— group.
E51. Compounds according to E1 wherein Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydroquinolinyl group, a tetrahydropyridinyl group, an azetidinyl group, a pyridinyl group, a pyrimidinyl group, a piperidinyl group, a morpholinyl group, a piperazinyl group, a cyclohexenyl group, a bicyclo[2.2.1]heptenyl group, a cyclopentenyl group, or a cyclohexyl group.
E52. Compounds according to E1 wherein Cy₂ represents an aryl group, a heteroaryl group, a cycloalkyl group, a cycloalkenyl group, or a heterocycloalkyl group which are substituted by 1, 2 or 3 groups selected from halogen, linear or branched (C₁-C₆)alkyl, linear or branched halo(C₁-C₆)alkyl, linear or branched (C₁-C₆)alkoxy, hydroxy, oxo, trifluoromethoxy, methoxyethoxy, —C(O)—OR′, phenyl, benzyl, (2,3,6-trifluorophenyl)methyl, pyridinyl, —CH₂-pyridinyl, —O-phenyl, —O-benzyl, —O—CH₂-cyclopropyl, —O-pyridinyl, —O—CH₂-pyridinyl, aryloxyalkyl, or heteroaryloxyalkyl, wherein R′ represents a linear or branched (C₁-C₆)alkyl group.
E53. Compounds according to E1 wherein Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydroquinolinyl group, a tetrahydropyridinyl group, an azetidinyl group, a pyridinyl group, a pyrimidinyl group, a piperidinyl group, a morpholinyl group, a piperazinyl group, a cyclohexenyl group, a bicyclo[2.2.1]heptenyl group, a cyclopentenyl group, or a cyclohexyl group which are substituted by 1, 2 or 3 groups selected from halogen, linear or branched (C₁-C₆)alkyl, linear or branched halo(C₁-C₆)alkyl, linear or branched (C₁-C₆)alkoxy, hydroxy, oxo, trifluoromethoxy, methoxyethoxy, —C(O)—OR′, phenyl, benzyl, (2,3,6-trifluorophenyl)methyl, pyridinyl, —CH₂-pyridinyl, —O-phenyl, —O-benzyl, —O—CH₂-cyclopropyl, —O-pyridinyl, —O—CH₂-pyridinyl, aryloxyalkyl, or heteroaryloxyalkyl, wherein R′ represents a linear or branched (C₁-C₆)alkyl group.
E54. Compounds according to E1 wherein Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydroquinolinyl group, a tetrahydropyridinyl group, an azetidinyl group, a pyridinyl group, a pyrimidinyl group, a piperidinyl group, a morpholinyl group, a piperazinyl group, a cyclohexenyl group, a bicyclo[2.2.1]heptenyl group, a cyclopentenyl group, or a cyclohexyl group which are substituted by 1, 2 or 3 groups selected from chlorine, fluorine, methyl, —CF₃, —CH₂—CF₃, methoxy, ethoxy, hydroxy, oxo, trifluoromethoxy, methoxyethoxy, —CO—O—C(CH₃)₃, phenyl, benzyl, (2,3,6-trifluorophenyl)methyl, pyridinyl, —CH₂-pyridinyl, —O-phenyl, —O-benzyl, —O—CH₂-cyclopropyl, —O-pyridinyl, —O—CH₂-pyridinyl, —CH₂—O-phenyl, —CH₂—O-pyridinyl, or —CH₂—O-thienopyridinyl.
E55. Compounds according to E1 wherein R₄ represents a —W₂-Cy₂ group wherein W₂ represents a bond and Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydropyridinyl group, an azetidinyl group, a cyclohexenyl group, a bicyclo[2.2.1]heptenyl group, or a cyclopentenyl group.
E56. Compounds according to E1 wherein R₄ represents a —W₂-Cy₂ group wherein W₂ represents a bond and Cy₂ represents a cyclopropyl group, a cyclobutyl group, a cyclopentyl group, a phenyl group, a pyrazolyl group, a thiazolyl group, an isoxazolyl group, a thienyl group, a furyl group, a benzofuranyl group, an indolyl group, a dihydrobenzofuranyl group, a tetrahydropyridinyl group, an azetidinyl group, a cyclohexenyl group, a bicyclo[2.2.1]heptenyl group, or a cyclopentenyl group which are substituted by 1, 2 or 3 groups selected from halogen, linear or branched (C₁-C₆)alkyl, linear or branched halo(C₁-C₆)alkyl, linear or branched (C₁-C₆)alkoxy, hydroxy, trifluoromethoxy, methoxyethoxy, phenyl, benzyl, (2,3,6-trifluorophenyl)methyl, pyridinyl, —CH₂-pyridinyl, —O-phenyl, —O-benzyl, —O—CH₂-cyclopropyl, —O-pyridinyl, —O—CH₂-pyridinyl, aryloxyalkyl, or heteroaryloxyalkyl.
E57. Compounds according to E1 wherein R₄ represents a —W₂-Cy₂ group wherein W₂ represents a linear or branched (C₁-C₄)alkylene group, a linear or branched (C₂-C₄)alkenylene group, or a linear or branched (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group.
E58. Compounds according to E1 wherein R₄ represents a —W₂-Cy₂ group wherein W₂ represents a linear or branched (C₁-C₄)alkylene group, a linear (C₂-C₄)alkenylene group, or a linear (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group.
E59. Compounds according to E1 wherein R₄ represents a —W₂-Cy₂ group wherein W₂ represents a linear or branched (C₁-C₄)alkylene group, a linear or branched (C₂-C₄)alkenylene group, or a linear or branched (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group which are substituted by 1, 2 or 3 groups selected from halogen, linear or branched (C₁-C₆)alkyl, linear or branched (C₁-C₆)alkoxy, oxo, —C(O)—OR′, or phenyl, wherein R′ represents a linear or branched (C₁-C₆)alkyl group.
E60. Compounds according to E1 wherein R₄ represents a —W₂-Cy₂ group wherein W₂ represents a linear or branched (C₁-C₄)alkylene group, a linear (C₂-C₄)alkenylene group, or a linear (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group which are substituted by 1, 2 or 3 groups selected from halogen, linear or branched (C₁-C₆)alkyl, linear or branched (C₁-C₆)alkoxy, oxo, —C(O)—OR′, or phenyl, wherein R′ represents a linear or branched (C₁-C₆)alkyl group.
E61. Compounds according to E1 wherein R₄ represents a —W₂-Cy₂ group wherein W₂ represents a linear or branched (C₁-C₄)alkylene group, a linear (C₂-C₄)alkenylene group, or a linear (C₂-C₄)alkynylene group and Cy₂ represents a cyclopentyl group, a phenyl group, an indolyl group, a pyridinyl group, a pyrimidinyl group, a tetrahydroquinolinyl group, a piperidinyl group, a morpholinyl group, a piperazinyl group, or a cyclohexyl group which are substituted by 1, 2 or 3 groups selected from chlorine, methyl, methoxy, oxo, —CO—O—C(CH₃)₃, or phenyl.
E62. Compounds according to E1 wherein R₄ represents a —W₃-L-Cy₃ group.
E63. Compounds according to E1 wherein W₃ represents a bond, a linear or branched (C₁-C₆)alkylene group, a linear or branched (C₂-C₈)alkynylene group, a linear or branched (C₁-C₆)alkoxylene group, a branched (C₁-C₄)hydroxyalkylene group, a linear or branched (C₁-C₄)haloalkylene group, or a —CH₂—CH(R_{4E})—CH₂— group.
E64. Compounds according to E1 wherein W₃ represents a bond, a —CH₂— group, a —(CH₂)₂— group, a —(CH₂)₃— group, a —(CH₂)₄— group, a —CH₂—CH(CH₃)—CH₂— group, a —CH₂—CH(CH₂—CH₃)—CH₂— group, a —CH₂—CH[CH(CH₃)₂]—CH₂— group, a —CH₂—CH(CH₃)—CH(CH₃)— group, a —CH₂—C(CH₃)₂—CH₂— group, a —O—(CH₂)₃— group, a —CH₂—CH(CH₂—OH)—CH₂— group, a —CH₂—CH(CH₂—OCH₃)—CH₂— group, a —CH₂—CF₂—CH₂— group, a —CH₂—CHF—CH₂— group, a —CH₂—CH(CH₂F)—CH₂— group, a —CH₂—CH(CHF₂)—CH₂— group, a —CH₂—CF(CH₃)—CH₂— group, a —CH₂—CH(C≡C—CH₂—CH₂—CH₃)—CH₂— group, a —C≡C—CH₂— group, a —C≡C—CH₂—CH₂— group, or a —CH₂—CH(R_{4E})—CH₂— group.
E65. Compounds according to E1 wherein W₃ represents a —CH₂—CH(CH₃)—CH₂— group.
E66. Compounds according to E1 wherein L represents —O—.
E67. Compounds according to E1 wherein L represents —S— or —SO₂—.
E68. Compounds according to E1 wherein Cy₃ represents a phenyl group, a thienopyridinyl group, a pyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, a benzothiazolyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a cycloheptapyridinyl group, a pyranopyridinyl group, a tetrahydronaphthyridinyl group, a tetrahydro-5,8-ethanoquinolinyl group, a dihydroquinolinyl group, a benzyl group, a —(CH₂)₂-phenyl group, a —(CH₂)₃-phenyl group, or a —(CH₂)₃-pyridinyl group.
E69. Compounds according to E1 wherein Cy₃ represents an aryl group, a heteroaryl group, a cycloalkyl group, a heterocycloalkyl group, an arylalkyl group, or a heteroarylalkyl group which are substituted by 1, 2 or 3 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, —NR′R″, cyclopropyl, 2,2-dimethylcyclopropyl, a linear or branched (C₁-C₆)hydroxyalkyl, hydroxy, oxo, or difluoromethoxy, wherein R′ and R″ independently of one another represent a hydrogen atom or a linear or branched (C₁-C₆)alkyl group.
E70. Compounds according to E1 wherein Cy₃ represents a phenyl group, a thienopyridinyl group, a pyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, a benzothiazolyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a cycloheptapyridinyl group, a pyranopyridinyl group, a tetrahydronaphthyridinyl group, a tetrahydro-5,8-ethanoquinolinyl group, a dihydroquinolinyl group, a benzyl group, a —(CH₂)₂-phenyl group, a —(CH₂)₃-phenyl group, or a —(CH₂)₃-pyridinyl group which are substituted by 1, 2 or 3 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, —NR′R″, cyclopropyl, 2,2-dimethylcyclopropyl, a linear or branched (C₁-C₆)hydroxyalkyl, hydroxy, oxo, or difluoromethoxy, wherein R′ and R″ independently of one another represent a hydrogen atom or a linear or branched (C₁-C₆)alkyl group.
E71. Compounds according to E1 wherein Cy₃ represents a phenyl group, a thienopyridinyl group, a pyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, a benzothiazolyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a cycloheptapyridinyl group, a pyranopyridinyl group, a tetrahydronaphthyridinyl group, a tetrahydro-5,8-ethanoquinolinyl group, a dihydroquinolinyl group, a benzyl group, a —(CH₂)₂-phenyl group, a —(CH₂)₃-phenyl group, or a —(CH₂)₃-pyridinyl group which are substituted by 1, 2 or 3 groups selected from fluorine, chlorine, methyl, ethyl, isopropyl, —CHF₂, —CF₃, —CH₂CF₃, ═CHF, methoxy, —NH₂, —NH(CH₃), cyclopropyl, 2,2-dimethylcyclopropyl, hydroxymethyl, hydroxy, oxo, or difluoromethoxy.
E72. Compounds according to E1 wherein R₄ represents a —W₃-L-Cy₃ group wherein W₃ represents a bond, L represents —O— and Cy₃ represents an arylalkyl group or a heteroarylalkyl group.
E73. Compounds according to E1 wherein R₄ represents a —W₃-L-Cy₃ group wherein W₃ represents a bond, L represents —O— and Cy₃ represents a —(CH₂)₃-phenyl group or a —(CH₂)₃-pyridinyl group.
E74. Compounds according to E1 wherein R₄ represents a —W₃-L-Cy₃ group wherein W₃ represents a linear or branched (C₁-C₆)alkylene group, L represents —O— and Cy₃ represents an aryl group, a heteroaryl group, a heterocycloalkyl group, or an arylalkyl group.
E75. Compounds according to E1 wherein R₄ represents a —W₃-L-Cy₃ group wherein W₃ represents a linear or branched (C₁-C₆)alkylene group, L represents —O— and Cy₃ represents a phenyl group, a pyridinyl group, a thienopyridinyl group, an indolyl group, a benzodioxolyl group, a tetrahydroindazolyl group, an indanyl group, a thienopyrimidinyl group, a pyrimidinyl group, a quinolinyl group, a pyrrolopyridinyl group, a furopyridinyl group, a tetrahydroquinolinyl group, a cyclopentapyridinyl group, an indazolyl group, a cyclopentapyrimidinyl group, a tetrahydroquinazolinyl group, a tetrahydropyranyl group, a benzimidazolyl group, a dihydroindolyl group, a benzopyranyl group, a pyridinonyl group, a hexahydropentalenopyridinyl group, a benzothiazolyl group, a cycloheptapyridinyl group, a pyranopyridinyl group, a tetrahydronaphthyridinyl group, a tetrahydro-5,8-ethanoquinolinyl group, a dihydroquinolinyl group a benzyl group, or a —(CH₂)₂-phenyl group.
E76. Compounds according to E1 wherein R₄ represents a —W₃-L-Cy₃ group wherein W₃ represents a —CH₂—CH(CH₃)—CH₂— group, L represents —O— and Cy₃ represents a tetrahydroquinolinyl group.
E77. Compounds according to E1 wherein R₄ represents a —W₃-L-Cy₃ group wherein W₃ represents a —CH₂—CH(CH₃)—CH₂— group, L represents —O— and Cy₃ represents a tetrahydroquinolinyl group which is substituted by 1 or 2 groups selected from halogen, preferably a fluorine atom, linear or branched (C₁-C₆)alkyl, preferably a methyl group, or hydroxy.
E78. Compounds according to E1 wherein R₄ represents

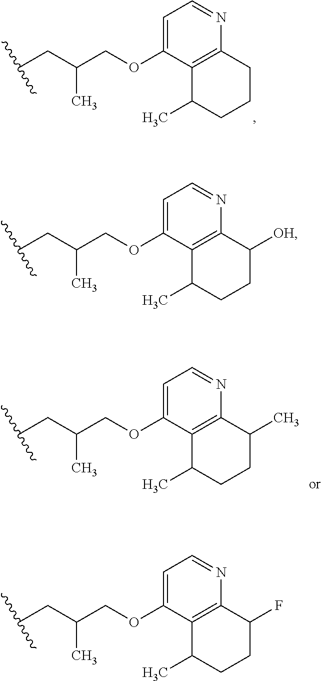 [SMILES: *CC(C)COc1ccnc2c1C(C)CCC2 ; *CC(C)COc1ccnc2c1C(C)CCC2O ; *CC(C)COc1ccnc2c1C(C)CCC2C ; *CC(C)COc1ccnc2c1C(C)CCC2F]

wherein the wavy line indicates the covalent attachment site to the spirocyclohexane scaffold.
E79. Compounds according to E1 wherein R₄ represents

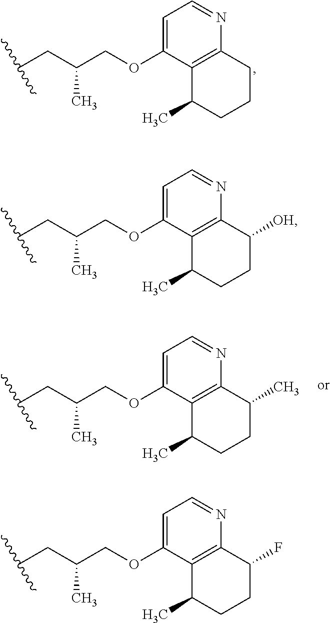 [SMILES: *C[C@@H](C)COc1ccnc2c1[C@H](C)CCC2 ; *C[C@@H](C)COc1ccnc2c1[C@H](C)CC[C@H]2O ; *C[C@@H](C)COc1ccnc2c1[C@H](C)CC[C@H]2C ; *C[C@@H](C)COc1ccnc2c1[C@H](C)CC[C@H]2F]

wherein the wavy line indicates the covalent attachment site to the spirocyclohexane scaffold.
E80. Compounds according to E1 wherein R₄ represents

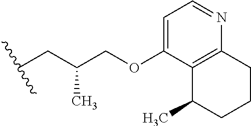 [SMILES: *C[C@@H](C)COc1ccnc2c1[C@H](C)CCC2]

wherein the wavy line indicates the covalent attachment site to the spirocyclohexane scaffold.
E81. Compounds according to E1 wherein R₄ represents a —W₃-L-Cy₃ group wherein W₃ represents a linear or branched (C₂-C₈)alkynylene group, L represents —O— and Cy₃ represents a phenyl group, or a thienopyridinyl group.
E82. Compounds according to E1 wherein R₄ represents a —W₃-L-Cy₃ group wherein W₃ represents a linear or branched (C₁-C₄)alkoxylene group, L represents —O— and Cy₃ represents a pyridinyl group, or a cyclopentapyridinyl group.
E83. Compounds according to E1 wherein R₄ represents a —W₃-L-Cy₃ group wherein W₃ represents a branched (C₁-C₄)hydroxyalkylene group, L represents —O— and Cy₃ represents a thienopyridinyl group, or a cyclopentapyridinyl group.
E84. Compounds according to E1 wherein R₄ represents a —W₃-L-Cy₃ group wherein W₃ represents a linear or branched (C₁-C₄)haloalkylene group, L represents —O— and Cy₃ represents a cyclopentapyridinyl group, a tetrahydroquinolinyl group, or a thienopyridinyl group.
E85. Compounds according to E1 wherein R₄ represents a —W₃-L-Cy₃ group wherein W₃ represents a —CH₂—CH(R_{4E})—CH₂— group, L represents —O— and Cy₃ represents a phenyl group, a thienopyridinyl group, a cyclopentapyridinyl group, a pyridinyl group, or a tetrahydroquinolinyl group.
E86. Compounds according to E1 wherein R_{4E} represents —Cy₄.
E87. Compounds according to E1 wherein R_{4E} represents —CH₂—O—Cy₄.
E88. Compounds according to E1 wherein Cy₄ represents a phenyl group, a benzyl group, a pyridinyl group, a thienopyridinyl group, a cyclopentapyridinyl group, or a —(CH₂)₂-morpholinyl group.
E89. Compounds according to E1 wherein R_{4E} represents —Cy₄ wherein Cy₄ represents a phenyl group, a benzyl group, or a pyridinyl group.
E90. Compounds according to E1 wherein R_{4E} represents —CH₂—O—Cy₄ wherein Cy₄ represents a thienopyridinyl group, a cyclopentapyridinyl group, or a —(CH₂)₂-morpholinyl group.
E91. Compounds according to E1 wherein R₄ represents a —W₄—NR_{4A}R_{4B} group.
E92. Compounds according to E1 wherein W₄ represents a —CH₂— group, a —(CH₂)₂— group, or a —CH₂—CH(CH₃)—CH₂— group.
E93. Compounds according to E1 wherein R_{4A} and R_{4B} independently of one another represent a hydrogen atom, a methyl group, a phenyl group, a pyridinyl group, a thienopyridinyl group, a tetrahydroquinolinyl group, or a benzyl group.
E94. Compounds according to E1 wherein R_{4A} represents a phenyl group, a pyridinyl group, a thienopyridinyl group, a tetrahydroquinolinyl group, or a benzyl group.
E95. Compounds according to E1 wherein R_{4B} represents a hydrogen atom, or a methyl group.
E96. Compounds according to E1 wherein R₄ represents a —CO—NR_{4C}R_{4D} group.
E97. Compounds according to E1 wherein R_{4C} and R_{4D} independently of one another represent a hydrogen atom, a methyl group, a benzyl group, a —(CH₂)₂-phenyl group, or a —(CH₂)₂-pyridinyl group.
E98. Compounds according to E1 wherein R_{4C} represents a benzyl group, a —(CH₂)₂-phenyl group, or a —(CH₂)₂-pyridinyl group.
E99. Compounds according to E1 wherein R_{4D} represents a hydrogen atom, or a methyl group.
E100. Compounds according to E1 wherein R₅ represents a hydrogen atom or a methyl group, preferably a hydrogen atom.
E101. Compounds according to E1 wherein the pair (R₄,R₅) represents an oxo group, or a cyclopentylidene group.
E102. Compounds according to E1 wherein the pair (R₄,R₅) together with carbon atoms to which they are attached forms a cyclopentyl ring.
E103. Compounds according to E1 wherein R₆ represents a phenyl group, a —SO₂-phenyl group, or a —W₅—O—Cy₅ group.
E104. Compounds according to E1 wherein W₅ represents a —(CH₂)₃— group, or a —CH₂—CH(CH₃)—CH₂— group, more preferably a —CH₂—CH(CH₃)—CH₂— group.
E105. Compounds according to E1 wherein Cy₅ represents a phenyl group, a thienopyridinyl group, a pyridinyl group, an indolyl group, or a tetrahydroquinolinyl group, more preferably a tetrahydroquinolinyl group, even more preferably, a tetrahydroquinolinyl group which is substituted by 1 or 2 groups selected from linear or branched (C₁-C₆)alkyl, preferably a methyl group.
E106. Compounds according to E1 wherein R₆ represents

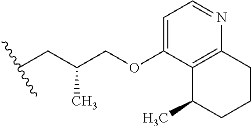 [SMILES: *C[C@@H](C)COc1ccnc2c1[C@H](C)CCC2]

wherein the wavy line indicates the covalent attachment site to the spirocyclohexane scaffold.
E107. Compounds according to E1 wherein R₇ represents a hydrogen atom, a methyl group, an ethyl group, a benzyl group, or a formyl group.
E108. Compounds according to E1 wherein R₈ represents a hydrogen atom, a methyl group, an ethyl group, or an isopropyl group, preferably a hydrogen atom.
E109. Compounds according to E1 wherein the pair (R₄,R₈) together with carbon atoms to which they are attached forms a cyclopropyl ring or a phenyl ring.
E110. Compounds according to E1 wherein R₉ represents a hydrogen atom.
E111. Compounds according to E1 wherein R₁₀ represents a hydrogen atom, a chlorine atom, a fluorine atom, a bromine atom, or a methyl group, preferably a hydrogen atom.
E112. Compounds according to E1 wherein the pair (R₇,R₁₀) together with the nitrogen atom to which they are attached forms a non-aromatic ring composed of 6 ring members.
E113. Compounds according to E1 wherein R₁₁ represents a hydrogen atom, a chlorine atom, a fluorine atom, a bromine atom, a methyl group, or a methoxy group.
E114. Compounds according to E1 wherein R₁₁ represents a hydrogen atom, a chlorine atom, a fluorine atom, or a methyl group, preferably a hydrogen atom.
E115. Compounds according to E1 wherein Ru represents a hydrogen atom, a fluorine atom, a bromine atom, a iodine atom, a chlorine atom, a methyl group, an ethyl group, a prop-1-enyl group, a —C≡CH group, a methoxy group, an ethoxy group, a propoxy group, an isopropoxy group, an isobutyloxy group, a 2-methoxypropan-2-yl group, a prop-2-en-1-yloxy group, a 2,2,2-trifluoroethoxy group, a methoxymethyl group, a methoxyethoxy group, a methoxypropoxy group, a hydroxy group, a hydroxymethyl group, a 1-hydroxyethyl group, an acetyl group, a formyl group, a —CH₂—O-tetrahydrofuranyl group, —Cy₆, or —O—Cy₇.
E116. Compounds according to E1 wherein R₁₂ represents a hydrogen atom, a methyl group, a methoxy group, an ethoxy group, an isopropoxy group, a 2-methoxypropan-2-yl group, a methoxymethyl group, a methoxyethoxy group or a 1-hydroxyethyl group.
E117. Compounds according to E1 wherein R₁₂ represents —Cy₆ which is selected from a phenyl group, a cyclopropyl group, a thienyl group, a pyrrolyl group, a —(CH₂)₂-phenyl group, a —CH(CH₃)-phenyl group, a —CH═CH-phenyl group, or a —CH═CH—CH₂-phenyl group.
E118. Compounds according to E1 wherein R₁₂ represents —Cy₆ which is selected from a phenyl group, a cyclopropyl group, a thienyl group, a pyrrolyl group, a —(CH₂)₂-phenyl group, a —CH(CH₃)-phenyl group, a —CH═CH-phenyl group, or a —CH═CH—CH₂-phenyl group which are substituted by 1, 2 or 3 groups selected from linear or branched (C₁-C₆)alkyl, linear or branched (C₁-C₆)alkoxy, linear or branched (C₁-C₆)alkoxy(C₁-C₆)alkyl, or hydroxy.
E119. Compounds according to E1 wherein R₁₂ represents —Cy₆ which is selected from a phenyl group, a cyclopropyl group, a thienyl group, a pyrrolyl group, a —(CH₂)₂-phenyl group, a —CH(CH₃)-phenyl group, a —CH═CH-phenyl group, or a —CH═CH—CH₂-phenyl group which are substituted by 1, 2 or 3 groups selected from methyl, methoxy, methoxymethyl, or hydroxy.
E120. Compounds according to E1 wherein R₁₂ represents —O—Cy₇ wherein Cy₇ represents a phenyl group, a cyclopentyl group, or a —CH₂-cyclopropyl group.
E121. Compounds according to E1 wherein the pair (R₁₁,R₁₂) together with the carbon atoms to which they are attached forms a non-aromatic ring composed of from 5 to 8 ring members, which may contain 1 or 2 oxygen atoms, wherein said ring may be substituted by R₁₈ and R₁₈′.
E122. Compounds according to E1 wherein the pair (R₁₁,R₁₂) together with the carbon atoms to which they are attached forms a non-aromatic ring as follows:

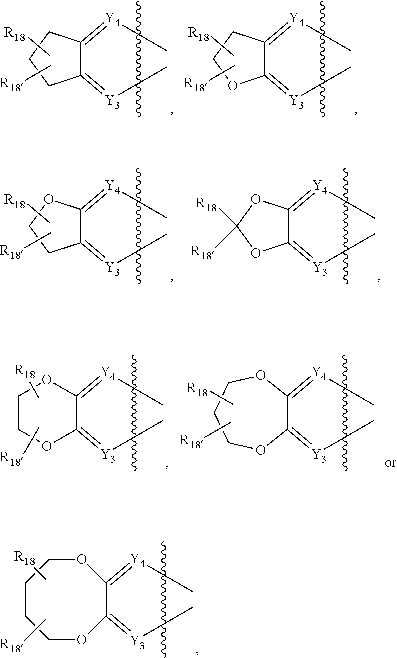

wherein R₁₈ and R₁₈′ are as defined for Formula (I).
E123. Compounds according to E1 wherein the pair (R₁₁,R₁₂) together with the carbon atoms to which they are attached forms a non-aromatic ring as follows:

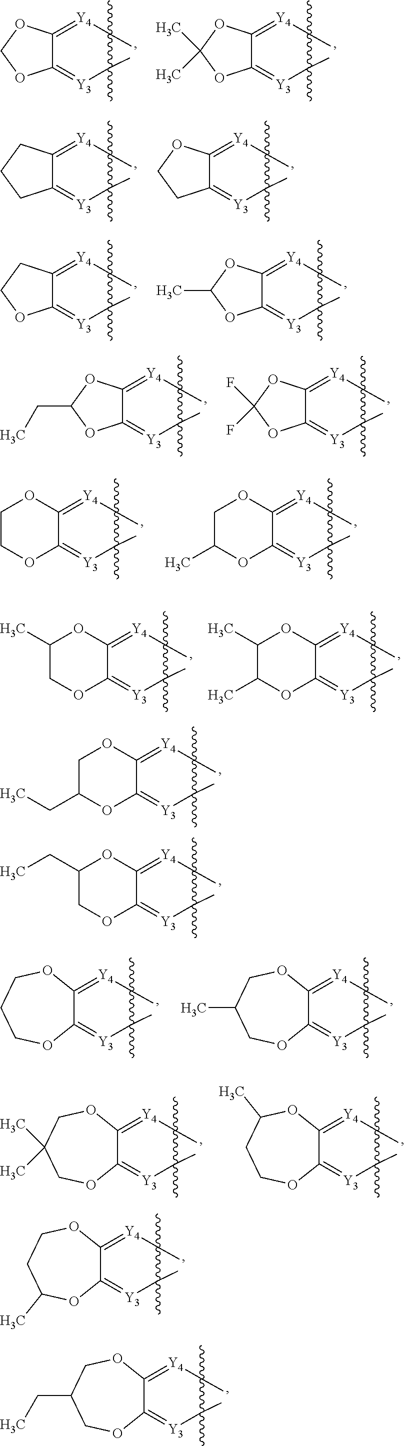

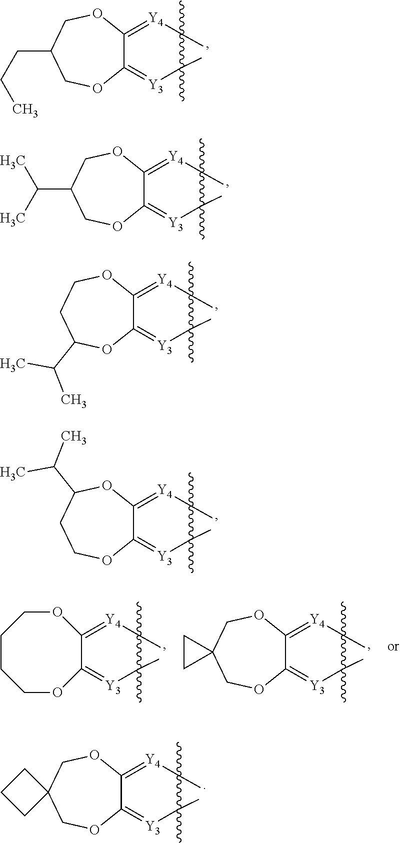

E124. Compounds according to E1 wherein the pair (R₁₁,R₁₂) together with the carbon atoms to which they are attached forms a non-aromatic ring as follows:

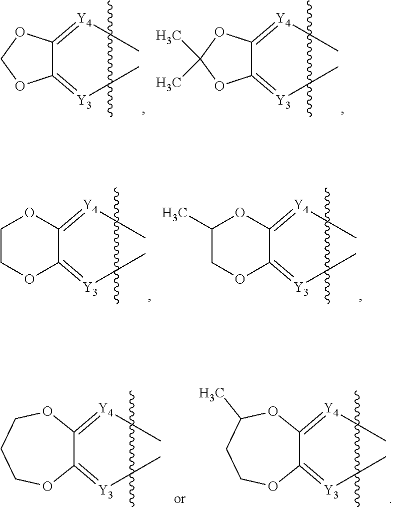

E125. Compounds according to E1 wherein the pair (R₁₁,R₁₂) together with the carbon atoms to which they are attached forms a non-aromatic ring as follows:

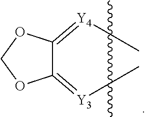

E126. Compounds according to E1 wherein R₁₃ represents a hydrogen atom, a fluorine atom, a bromine atom, or a methyl group, preferably a hydrogen atom.
E127. Compounds according to E1 wherein R₁₄ and R₁₅ represent a hydrogen atom.
E128. Compounds according to E1 wherein R₁₆ represents a —O—R₃ group.
E129. Compounds according to E1 wherein R₁₆ represents a —NR₁₇R₁₇′ group.
E130. Compounds according to E1 wherein R₁₆ represents a —NH₂ group, a —NH—CH₃ group, a —N(CH₃)₂ group, a —NH—SO₂—CF₃ group, or a —NH—SO₂—CH₃ group.
E131. Compounds according to E1 wherein R₁₇ and R₁₇′ independently of one another, represent a hydrogen atom, a methyl group, a —SO₂—CF₃ group, or a —SO₂—CH₃ group.
E132. Compounds according to E1 wherein R₁₇ represents a hydrogen atom, a methyl group, a —SO₂—CF₃ group, or a —SO₂—CH₃ group.
E133. Compounds according to E1 wherein R₁₇′ represents a hydrogen atom or a methyl group.
E134. Compounds according to E1 wherein R₁₈ and R₁₈′ independently of one another, represent a hydrogen atom, a fluorine atom, a methyl group, an ethyl group, a n-propyl group or an isopropyl group.
E135. Compounds according to E1 wherein R₁₈ represents a methyl group, an ethyl group, a n-propyl group or an isopropyl group and R₁₈′ represents a hydrogen atom.
E136. Compounds according to E1 wherein R₁₈ and R₁₈′ represent both a methyl group.
E137. Compounds according to E1 wherein R₁₈ and R₁₈′ represent both a fluorine atom.
E138. Compounds according to E1 wherein the pair (R₁₈,R₁₈′) together with the carbon atoms to which they are attached forms a cyclopropyl ring or a cyclobutyl ring.
E139. Compounds according to E24, wherein:
   X represents —N(R₂)—,
   R₁ represents an aryl group,
   R₂, R₈, R₉, R₁₀ and R₁₃ represent a hydrogen atom,
   or the pair (R₈,R₉) represents an oxo group,
   R₆ represents an aryl group, a —SO₂-aryl group, or a —W₅—O—Cy₅ group, wherein:
      W₅ represents a linear or branched (C₁-C₄)alkylene group,
      Cy₅ represents an aryl group, or a heteroaryl group,
   R₁₁ represents a hydrogen atom, a halogen atom, or a linear or branched (C₁-C₆)alkyl group,
   R₁₂ represents a hydrogen atom, a linear or branched (C₁-C₆)alkoxy group, or a linear or branched (C₁-C₆)alkoxy(C₁-C₆)alkyl group,
   or the pair (R₁₁,R₁₂) together with the carbon atoms to which they are attached forms a non-aromatic ring composed of from 5 to 8 ring members, which may contain 1 or 2 oxygen atoms
   R₁₆ represents a —O—R₃ group wherein R₃ represents a hydrogen atom.
E140. Compounds according to E26. wherein:
   X represents —S—, —O—, —CH₂— or —N(R₂)—,
   R₁ represents an aryl group or a heteroaryl group,
   R₂ represents a hydrogen atom,
   or the pair (R₁,R₂) together with the nitrogen atom to which they are attached forms a non-aromatic or aromatic mono- or bicyclic ring composed of from 5 to 12 ring members, which may contain in addition to the nitrogen a second heteroatom selected from oxygen, sulphur and nitrogen, wherein said ring may be substituted by from 1 to 2 groups representing a hydrogen atom, a halogen atom, or a linear or branched (C₁-C₆)alkyl group,
   R₁₆ represents a —O—R₃ group,
   R₃ represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)(OR_{3A})₂, —W₁—SO₂—OR_{3A}, —W₁—Cy₁, wherein:
      W₁ represents a bond or a linear or branched (C₁-C₄)alkylene group,
      R_{3A} and R_{3B} independently of one another, represent a hydrogen atom, a linear or branched (C₁-C₆)alkyl group, a linear or branched (C₁-C₆)alkoxy(C₁-C₆)alkyl group, a cycloalkyl group, or the pair (R_{3A},R_{3B}) together with the nitrogen atom to which they are attached forms a non-aromatic ring composed of from 4 to 7 ring members, which may contain in addition to the nitrogen a second heteroatom selected from oxygen and nitrogen,
      Cy₁ represents an aryl group or a heteroaryl group,
   R₄ represents a hydrogen atom, a linear or branched (C₁-C₆)alkyl group, a linear or branched (C₂-C₆)alkenyloxy group, a linear or branched (C₁-C₆)alkoxy(C₁-C₆)alkyl group, a linear or branched (C₁-C₆)alkoxy(C₁-C₆)haloalkyl group, a —W₂-Cy₂ group, a —W₃-L-Cy₃ group, a —W₄—NR_{4A}R_{4B} group, or a —CO—NR_{4C}R_{4D} group, wherein:
      W₂ represents a bond, a linear or branched (C₁-C₆)alkylene group,
      W₃ represents a bond, a linear or branched (C₁-C₆)alkylene group, a linear or branched (C₁-C₆)alkoxylene group, a linear or branched (C₁-C₄)hydroxyalkylene group, a linear or branched (C₁-C₄)haloalkylene group, or a —CH₂—CH(R_{4E})—CH₂— group,
      W₄ represents a linear or branched (C₁-C₄)alkylene group,
      L represents —O—, —S—, or —SO₂—,
      R_{4A} and R_{4B} independently of one another represent a hydrogen atom, or a heteroaryl group,
      R_{4C} and R_{4D} independently of one another represent a hydrogen atom, a linear or branched (C₁-C₆)alkyl group, an arylalkyl group, or a heteroarylalkyl group,
      R_{4E} represents —Cy₄ or —CH₂—O—Cy₄,
      Cy₂ represents an aryl group, a heteroaryl group, a cycloalkyl group, or a heterocycloalkyl group,
      Cy₃ represents an aryl group, a heteroaryl group, a heterocycloalkyl group, an arylalkyl group, or a heteroarylalkyl group,
      Cy₄ represents an aryl group, a heteroaryl group, an arylalkyl group, or a heterocycloalkylalkyl group,
   R₅ represents a hydrogen atom or a linear or branched (C₁-C₆)alkyl group,
   or the pair (R₄,R₅) represents a cycloalkylidene group
   or the pair (R₄,R₅) together with carbon atoms to which they are attached forms a non-aromatic ring composed of from 3 to 7 ring members,
   R₈ represents a hydrogen atom, a linear or branched (C₁-C₆)alkyl group,
   or the pair (R₄,R₈) together with carbon atoms to which they are attached forms a non-aromatic or aromatic ring composed of from 3 to 7 ring members,
   R₉ represents a hydrogen atom,
   R₁₀ represents a hydrogen atom, a halogen atom, or a linear or branched (C₁-C₆)alkyl group,
   R₁₁ represents a hydrogen atom, a halogen atom, or a linear or branched (C₁-C₆)alkyl group, or a linear or branched (C₁-C₆)alkoxy group,
   R₁₂ represents a hydrogen atom, a halogen atom, a linear or branched (C₁-C₆)alkyl group, a linear or branched (C₁-C₆)alkoxy group, a linear or branched (C₁-C₆)alkenyloxy group, a linear or branched halo(C₁-C₆)alkyloxy group, a linear or branched (C₁-C₆)alkoxy(C₁-C₆)alkyl group, a linear or branched (C₁-C₆)alkoxy(C₁-C₆)alkoxy group, a hydroxy group, a linear or branched hydroxy(C₁-C₆)alkyl group, an acetyl group, a formyl group, a —CH₂—O-tetrahydrofuranyl group, —Cy₆, or —O—Cy₇, wherein:
      Cy₆ represents an aryl group, a heteroaryl group, a cycloalkyl group, or an arylalkyl group,
      Cy₇ represents an aryl group, a cycloalkyl group, or a cycloalkylalkyl group,
   or the pair (R₁₁,R₁₂) together with the carbon atoms to which they are attached forms a non-aromatic ring composed of from 5 to 8 ring members, which may contain 1 or 2 oxygen atoms wherein said ring may be substituted by R₁₈ and R₁₈′,
   R₁₃ represents a hydrogen atom, a halogen atom, or a linear or branched (C₁-C₆)alkyl group;
   R₁₈ and R₁₈′ independently of one another, represent a hydrogen atom, a halogen atom, or a linear or branched (C₁-C₆)alkyl group,
   or the pair (R₁₈,R₁₈′) together with the carbon atoms to which they are attached forms a cyclopropyl ring or a cyclobutyl ring.
E141. Compounds according to E26. or E140. wherein compounds of Formula (I-e) are:

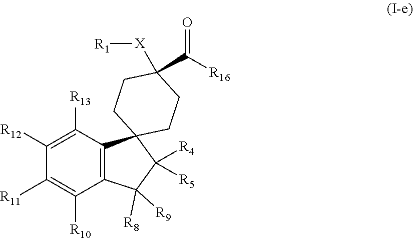

wherein R₁, R₄, R₅, R₈, R₉, R₁₀, R₁₁, R₁₂, R₁₃, R₁₆ and X are as defined in E26. or E140.
E142. Compounds according to E141. wherein compounds of Formula (I-e) are:

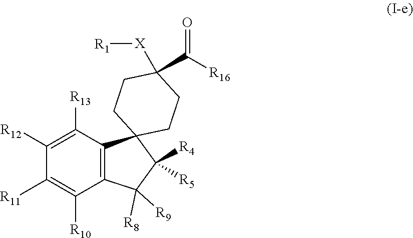

wherein R₁, R₄, R₅, R₈, R₉, R₁₀, R₁₁, R₁₂, R₁₃, R₁₆ and X are as defined in E141.
E143. Compounds according to E1 which are:
(1s,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid;
(1s,4S)-4-(3-chloro-4-fluoroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid;
(1s,4S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid;
(1s,4S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid;
(1r,2′S,4S)-4-(3-chloro-2-methylanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid;
(1r,2′S,4S)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-[(propan-2-yl)oxy]-2′,3′-dihydrospiro[cyclohexane-1,1′-indene]-4-carboxylic acid;
(1r,2′S,4S)-4-(3-chloroanilino)-6′-(2-methoxyethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-5′-chloro-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,4S,8'S)-4-(3-chloroanilino)-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid;
(1r,4S,7'S)-4-(3-chloroanilino)-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,4S,6'S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-2-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-2-methylanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloroanilino)-2′,2′-dimethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,2′S,4S)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′R,4R)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(2-methoxypropan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,4S,6'S)-4-(3-chloroanilino)-6′-[(2R)-3-{[(5R,8R)-8-hydroxy-5-methyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid;
(1r,4S,7'S)-4-(3-chloroanilino)-7′-[(2R)-3-{[(5R,8R)-8-hydroxy-5-methyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylic acid;
(1r,3'S,4S,7'S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,3′R,4S,7'S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,4S,4'S,8'S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid;
(1r,4S,4′R,8'S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid.
   E144. A compound of Formula (IIIA):

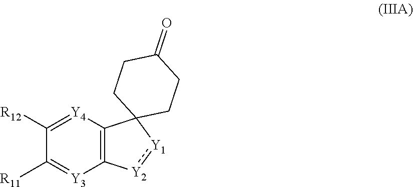

wherein R₁₁, R₁₂, Y₁, Y₂, Y₃, Y₄ and [symbol] are as defined in E1, as synthesis intermediate for the preparation of compounds of Formula (I) according to E1.
E145. A compound of Formula (IIIA) according to E144. which is 6′-bromo-2′H-spiro[cyclohexane-1,5′-indeno[5,6-d][1,3]dioxol]-4-one.
E146. A compound of Formula (IIIA) according to E144. or E145. for use as synthesis intermediate for the preparation of compounds of Formula (I) according to E1.
E147. A compound of Formula (VA):

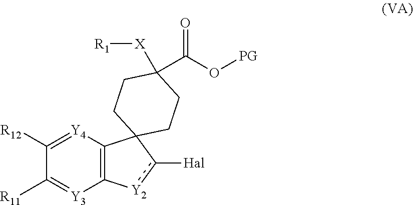

wherein R₁, R₁₁, R₁₂, X, Y₂, Y₃, Y₄ and [symbol] are as defined in E1, and Hal represents a halogen atom and PG represents a protecting group of the carboxylic acid function, as synthesis intermediate for the preparation of compounds of Formula (I) according to E1.
E148. A compound of Formula (VA) according to E147. which is:
methyl (1s,4s)-2′-bromo-4-(3-chloroanilino)spiro[cyclohexane-1,1′-indene]-4-carboxylate;
methyl (1s,4s)-6′-bromo-4-(3-chloroanilino)-2′H-spiro[cyclohexane-1,5′-indeno[5,6-d][1,3]dioxole]-4-carboxylate.
   E149. Pharmaceutical composition comprising a compound of Formula (I) according to any one of embodiments E1 to E143. or an addition salt thereof with a pharmaceutically acceptable acid or base in combination with one or more pharmaceutically acceptable excipients.
   E150. Pharmaceutical composition according to E149. for use as anti-apoptotic inhibitors.
   E151. Pharmaceutical composition according to E149. for use in the treatment of cancer and of auto-immune and immune system diseases.
   E152. Pharmaceutical composition according to E151. wherein the cancer is an haematological malignancy or a solid tumor.
   E153. Pharmaceutical composition according to E151. or E152. wherein the cancer is chemo-resistant or radio-resistant.
   E154. Pharmaceutical composition according to E152. wherein the haematological malignancy is myeloma, especially multiple myeloma, lymphoma, especially Non-Hodgkin Lymphoma (NHL) and Diffuse Large B-cell Lymphoma (DLBCL), and leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML).
   E155. Pharmaceutical composition according to E152. wherein the solid tumor is selected from bladder, brain, breast, uterus, oesophagus and liver cancers, colorectal cancer, renal cancer, melanoma, ovarian cancer, prostate cancer, pancreatic cancer and lung cancer, especially non-small-cell lung cancer and small-cell lung cancer.
   E156. Pharmaceutical composition according to E151. wherein the auto-immune and immune system diseases are rheumatoid arthritis (RA) and systemic lupus erythematosus (SLE).
   E157. Compound of Formula (I) according to any one of embodiments E1 to E143. or an addition salt thereof with a pharmaceutically acceptable acid or base, for use as anti-apoptotic inhibitors.
   E158. Compound of Formula (I) according to any one of embodiments E1 to E143. or an addition salt thereof with a pharmaceutically acceptable acid or base, for use in the treatment of cancer and of auto-immune and immune system diseases.
   E159. Compound of Formula (I) according to any one of embodiments E1 to E143., or an addition salt thereof with a pharmaceutically acceptable acid or base, for use according to E158. wherein the cancer is an haematological malignancy or a solid tumor.
   E160. Compound of Formula (I) according to any one of embodiments E1 to E143., or an addition salt thereof with a pharmaceutically acceptable acid or base, for use according to E158. wherein the cancer is chemo-resistant or radio-resistant.
   E161. Compound of Formula (I) according to any one of embodiments E1 to E143., or an addition salt thereof with a pharmaceutically acceptable acid or base, for use according to E159. wherein the haematological malignancy is myeloma, especially multiple myeloma, lymphoma, especially Non-Hodgkin Lymphoma (NHL) and Diffuse Large B-cell Lymphoma (DLBCL), and leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML).
   E162. Compound of Formula (I) according to any one of embodiments E1 to E143., or an addition salt thereof with a pharmaceutically acceptable acid or base, for use according to E159. wherein the solid tumor is selected from bladder, brain, breast, uterus, oesophagus and liver cancers, colorectal cancer, renal cancer, melanoma, ovarian cancer, prostate cancer, pancreatic cancer and lung cancer, especially non-small-cell lung cancer and small-cell lung cancer.
   E163. Compound of Formula (I) according to any one of embodiments E1 to E143., or an addition salt thereof with a pharmaceutically acceptable acid or base, for use according to E158. wherein the auto-immune and immune system diseases are rheumatoid arthritis (RA) and systemic lupus erythematosus (SLE).
   E164. Combination of a compound of Formula (I) according to any one of embodiments E1 to E143. with anti-cancer agents selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies.
   E165. Pharmaceutical composition comprising a combination according to E164. in combination with one or more pharmaceutically acceptable excipients.
   E166. Combination according to E165. for use in the treatment of cancer.
   E167. Combination according to E166. wherein the cancer is chemo-resistant or radio-resistant.
   E168. Combination according to E166. or E167. wherein the cancer is selected from myeloma, especially multiple myeloma, lymphoma, especially Non-Hodgkin Lymphoma (NHL) and Diffuse Large B-cell Lymphoma (DLBCL), leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML), bladder, brain, breast, uterus, oesophagus and liver cancers, colorectal cancer, renal cancer, melanoma, ovarian cancer, prostate cancer, pancreatic cancer and lung cancer, especially non-small-cell lung cancer and small-cell lung cancer.
   E169. Compound of Formula (I) according to any one of embodiments E1 to E143. for use in the treatment of cancer requiring radiotherapy.

Pharmacological studies of the compounds of the invention have shown that they have pro-apoptotic properties. The ability to reactivate the apoptotic process in cancerous cells is of major therapeutic interest in the treatment of cancer and of immune and auto-immune diseases.

The present invention relates also to pharmaceutical compositions comprising at least one compound of Formula (I) or an addition salt thereof with a pharmaceutically acceptable acid or base in combination with one or more pharmaceutically acceptable excipients. In particular, these pharmaceutical compositions are interesting for use as anti-apoptotic inhibitors, particularly, in the treatment of cancer (haematological malignancy and solid tumor) and of auto-immune and immune system diseases. Particularly, these pharmaceutical compositions are interesting for use as anti-apoptotic inhibitors in the treatment of cancer chemo-resistant or radio-resistant. Preferably, these pharmaceutical compositions can be used in the treatment of cancer (haematological malignancy and solid tumor) and of auto-immune and immune system diseases selected from myeloma, especially multiple myeloma, lymphoma, especially Non-Hodgkin Lymphoma (NHL) and Diffuse Large B-cell Lymphoma (DLBCL), leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML), bladder, brain, breast, uterus, oesophagus and liver cancers, colorectal cancer, renal cancer, melanoma, ovarian cancer, prostate cancer, pancreatic cancer, lung cancer, especially non-small-cell lung cancer and small-cell lung cancer, rheumatoid arthritis (RA) or systemic lupus erythematosus (SLE).

Furthermore, the present invention relates also to the combination of a compound of Formula (I) with an anticancer agent selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies, and also to pharmaceutical compositions comprising that type of combination and their use in the manufacture of medicaments for use in the treatment of cancer, particularly, haematological malignancy and solid tumors selected from myeloma, especially multiple myeloma, lymphoma, especially Non-Hodgkin Lymphoma (NHL) and Diffuse Large B-cell Lymphoma (DLBCL), leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML), bladder, brain, breast, uterus, osophagus and liver cancers, colorectal cancer, renal cancer, melanoma, ovarian cancer, prostate cancer, pancreatic cancer and lung cancer, especially non-small-cell lung cancer and small-cell lung cancer.

Alternatively, the compounds of the invention may be linked to monoclonal antibodies. Antibody Drug Conjugates (ADCs) represent a class of therapeutics that is formed by chemically linking a cytotoxic drug to a monoclonal antibody through a linker. The monoclonal antibody of an ADC selectively binds to a target antigen of a cell (e.g. cancer cell) and releases the drug into the cell or in the cell environment. ADCs have therapeutic potential because they combine the specificity of the antibody and the cytotoxic potential of the drug. Nonetheless, developing ADCs as therapeutic agents has thus far met with limited success owing to a variety of factors such as unfavorable toxicity profiles, low efficacies and poor pharmacological parameters. Accordingly, there is still a need for new ADCs that overcome these problems and can selectively deliver Mcl-1 inhibitors to target cancer cells.

In another aspect, the compounds of the invention may be linked to monoclonal antibodies or fragments thereof or linked to scaffold proteins that can be related or not to monoclonal antibodies. Antibody fragments must be understood as fragments of Fv, scFv, Fab, F(ab′)2, F(ab′), scFv-Fc type or diabodies, which generally have the same specificity of binding as the antibody from which they are descended. According to the present invention, antibody fragments of the invention can be obtained starting from antibodies by methods such as digestion by enzymes, such as pepsin or papain, and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc.

Scaffold proteins that can be related or not to monoclonal antibodies are understood to mean a protein that contains or not an immunoglobulin fold and that yields a binding capacity similar to a monoclonal antibody. The man skilled in the art knows how to select the protein scaffold.

More particularly, it is known that, to be selected, such a scaffold should display several features as follows (Skerra, J. Mol. Recogn. 2000, 13, 167-187): phylogenetically good conservation, robust architecture with a well-known three-dimensional molecular organization (such as, for example, crystallography or NMR), small size, no or only a low degree of post-translational modifications, easy to produce, express and purify. Such a protein scaffold can be, but without limitation, a structure selected from the group consisting in fibronectin and preferentially the tenth fibronectin type III domain (FNfn10), lipocalin, anticalin (Skerra, J. Biotechnol. 2001, 74, 257-75), the protein Z derivative from the domain B of staphylococcal protein A, thioredoxin A or any protein with a repeated domain such as an “ankyrin repeat” (Kohl et al, PNAS 2003, 100, 1700-1705), “armadillo repeat”, “leucine-rich repeat” or “tetratricopeptide repeat”. There could also be mentioned a scaffold derivative from toxins (such as, for example, scorpion, insect, plant or mollusc toxins) or protein inhibitors of neuronal nitric oxide synthase (PIN).

### EXAMPLES

The compounds of the present disclosure can be prepared in a number of ways well known to those skilled in the art of organic synthesis. By way of example, compounds of the invention can be synthesized using the methods described below, together with synthetic methods known in the art of synthetic organic chemistry, or variations thereon as appreciated by those skilled in the art. It is understood that at any moment considered appropriate during the processes described below, some groups (halogen, hydroxy, amino . . . ) of the starting reagents or of the synthesis intermediates can be protected, subsequently deprotected and functionalized, as required by the synthesis. Preferred methods include but are not limited to those methods described below. Compounds of the present invention can be synthesized by following the steps outlined in General Schemes 1, 2, 3, 4, 5, 6, 7 and 8 which comprise different sequences of preparing intermediates IIA, IIB, IIC, IIIA, IIIC, IVA, IVD, VA, VA′, VB, VIA, VIA′, VIB, VIB′, VIB″, VIIA and VIIA′. Starting materials IA, IB, IC and ID are either commercially available or made by known procedures in the reported literature or as illustrated.

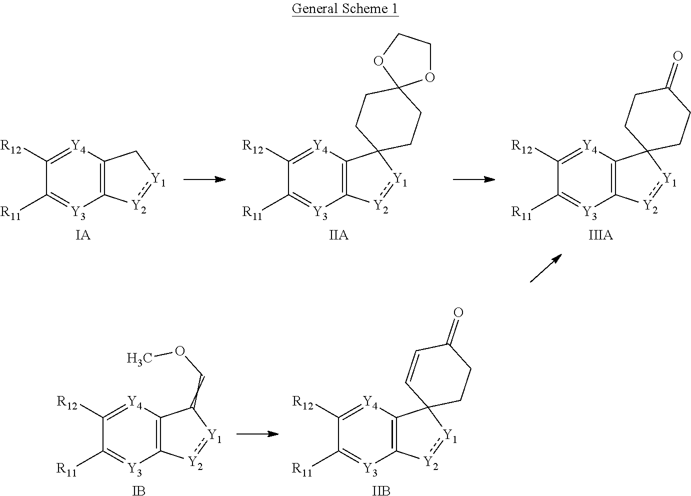

wherein Y₁, Y₂, Y₃, Y₄, R₁₁, R₁₂ and [symbol] are as defined in Formula (I).

The general way of preparing key-intermediate ketone IIIA containing spirocyclohexane scaffold by using intermediates IIA and IIB is outlined in General Scheme 1. Spirocyclization of starting material IA with (1,3-dioxolane-2,2-diyl)di(ethane-2,1-diyl)methanesulfonate or 2,2-bis(2-bromoethyl)-1,3-dioxolane using a strong base (such as NaH or LIHDMS) at low temperatures and ketal cleavage under acidic conditions provides intermediate ketone IIIA. Alternatively, preparation of key-intermediate ketone IIIA can be performed by spirocyclization of starting material IB with methyl vinyl ketone (also known as but-3-en-2-one) under acidic conditions (for example, using PTSA) at elevated temperatures providing intermediate IIB. Finally, hydrogenation of IIB using catalytic amount of Pd/C and dihydrogen (or other known reagents for hydrogenation reaction) provides IIIA.

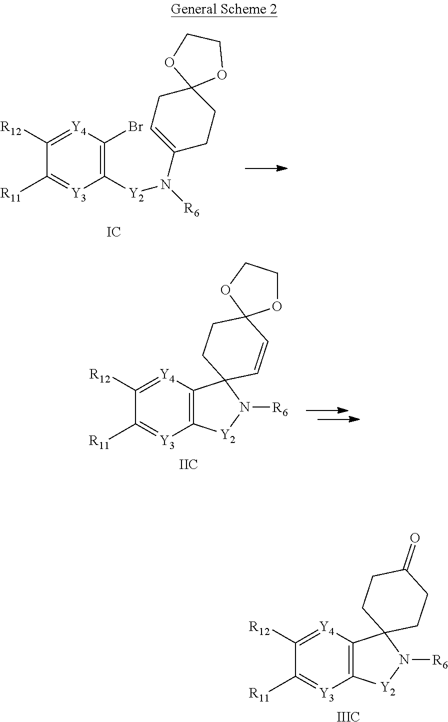

wherein Y₂, Y₃, Y₄, R₆, R₁₁ and R₁₂ are as defined in Formula (I).

Even if spirocyclohexane derivatives wherein Y₁ represents —N(R₆)— can be prepared through synthetic pathway as outlined in General Scheme 1 above, an alternative way for preparing these specific compounds is outlined in General Scheme 2. Spirocyclization of starting material IC using a Pd salt (such as Pd(OAc)₂) at high temperatures provides intermediate IIC. Finally, hydrogenation of IIC using catalytic amount of Pd/C in presence of ammonium formate followed by ketal cleavage under acidic conditions provides intermediate ketone IIIC.

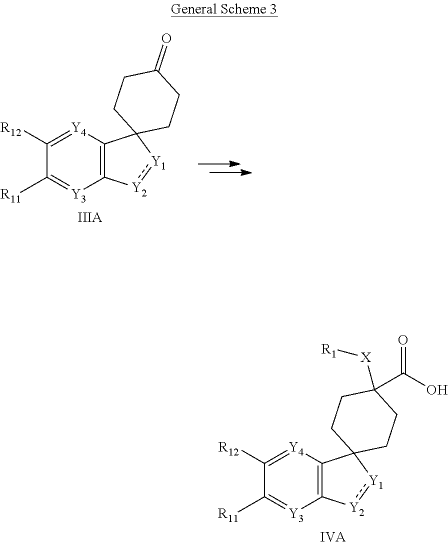

wherein X, Y₁, Y₂, Y₃, Y₄, R₁, R₁₁, R₁₂ and [symbol] are as defined in Formula (I).

The general way of preparing IVA containing spirocyclohexane scaffold by using intermediate ketone IIIA is outlined in General Scheme 3. In one embodiment, ketone's functionalization was performed via Bargellini reaction using appropriate reactant R₁—X—H in presence of NaOH and CHCl₃ at low temperatures providing IVA.

In a particular embodiment for the preparation of IVA wherein X represents —N(R₂)—, ketone IIIA is subjected to Strecker reaction using appropriate reactant R₁—NH₂ in presence of cyanide salt yielding a cyano intermediate which is transformed to the corresponding amide derivative and the latter is finally hydrolyzed to yield IVA.

In another embodiment for the preparation of IVA wherein X represents —N(R₂)—, ketone IIIA is subjected to Bucherer-Bergs reaction using ammonium carbonate and potassium cyanide at elevated temperatures yielding a hydantoin intermediate which is then hydrolyzed to provide an amino acid intermediate and the latter is finally subjected to Ullmann reaction in presence of copper and R₁—Z wherein Z is a halogen atom to yield IVA.

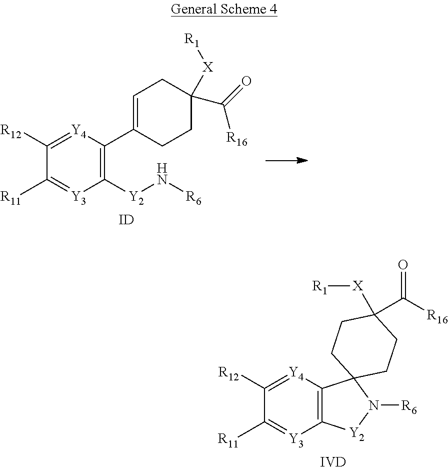

wherein X, Y₂, Y₃, Y₄, R₁, R₆, R₁₁, R₁₂ and R₁₆ are as defined in Formula (I).

Even if spirocyclohexane derivatives wherein Y₁ represents —N(R₆)— can be prepared through synthetic pathway as outlined in General Scheme 3 above, an alternative way for preparing these specific compounds is outlined in General Scheme 4. Spirocyclization of starting material ID under acidic conditions provides IVD.

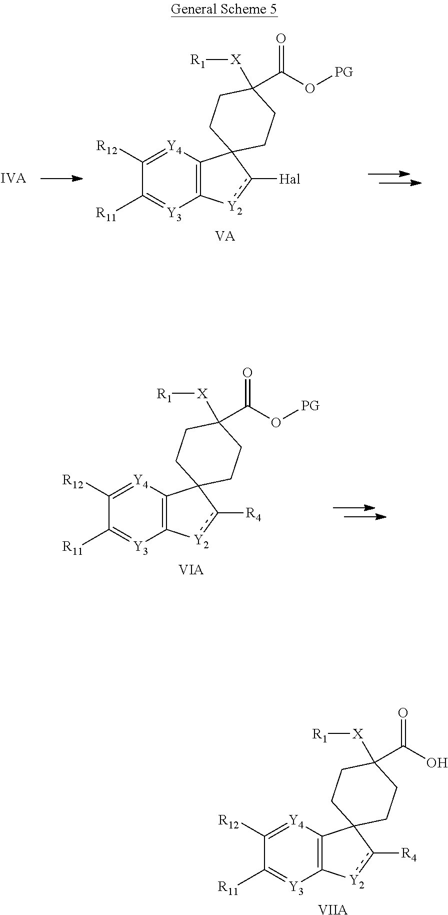

wherein X, Y₂, Y₃, Y₄, R₁, R₄, R₁₁, R₁₂ and [symbol] are as defined in Formula (I), and Hal represents a halogen atom and PG represents a protecting group of the carboxylic acid function.

In one preferred embodiment, a synthetic pathway for preparing VIIA is outlined in General Scheme 5. Starting material IVA wherein Y₁ represents —C(Hal)= or —C(Hal)(R₅)— and Hal represents a halogen atom, was esterified to provide key-intermediate VA wherein PG represents a protecting group of the carboxylic acid function such as methyl ester, ethyl ester, etc. Then, R₄ group was introduced according to classical chemical reactions using the corresponding reactants (for example, metal-catalyzed cross coupling using R₄—ZnBr reactant such as Negishi reaction) to provide VIA. In one embodiment, when [symbol] represents a double bond, an intermediate hydrogenation step of indene VIA can be performed to provide corresponding indane. Finally, VIIA is obtained after removal of carboxylic acid protecting group (for example, by ester hydrolysis).

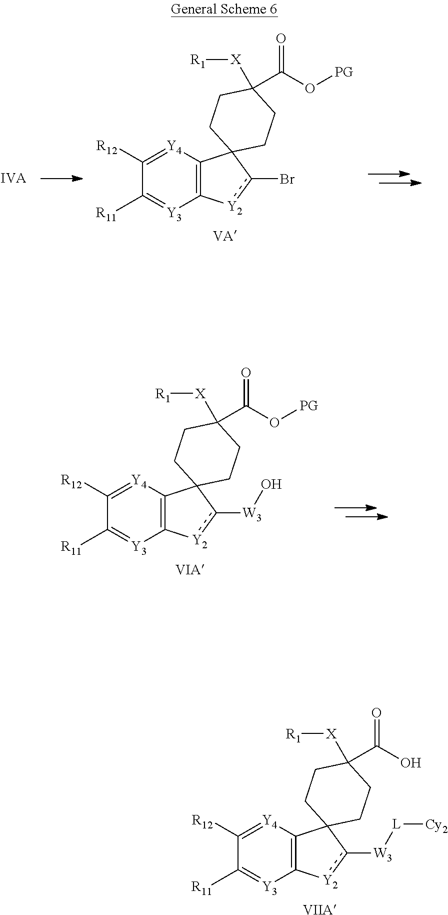

wherein Cy₃, L, W₃, X, Y₂, Y₃, Y₄, R₁, R₁₁, R₁₂ and [symbol] are as defined in Formula (I), and PG represents a protecting group of the carboxylic acid function.

In one preferred embodiment, a synthetic pathway for preparing VIIA′ is outlined in General Scheme 6. Starting material IVA wherein Y₁ represents —C(Br)═ or —C(Br)(R₅)— was esterified to provide VA′ wherein PG represents a protecting group of the carboxylic acid function such as methyl ester, ethyl ester, etc. Metal-catalyzed cross-coupling reaction in presence of W₃—OH reactant was performed to provide VIA′. In one embodiment, when [symbol] represents a double bond, an intermediate hydrogenation step of indene VIA′ is possible to provide corresponding indane. Finally, VIIA′ is obtained after coupling Cy₃-L-H on intermediate VIA′ (for example, through Mitsunobu reaction) and removal of carboxylic acid protecting group (for example, by ester hydrolysis).

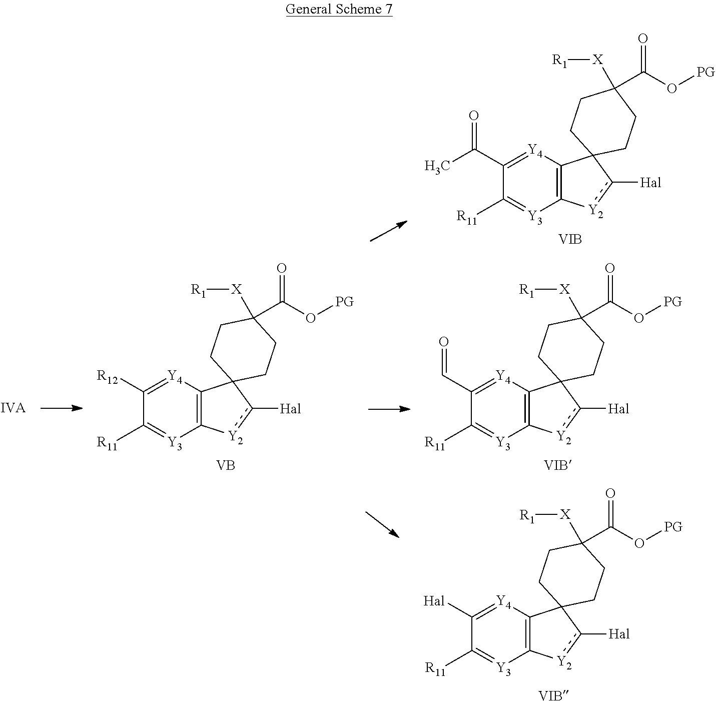

wherein X, Y₂, Y₃, Y₄, R₁, R₁₁ and [symbol] are as defined in Formula (I), and Hal represents a halogen atom and PG represents a protecting group of the carboxylic acid function.

In one another embodiment, a synthetic pathway for preparing VIB, VIB′ and VIB″ is outlined in General Scheme 7. Starting material IVA wherein Y₁ represents —C(Hal)= or —C(Hal)(R₅)—, R₁₂ represents a hydrogen atom and Hal represents a halogen atom, was esterified to provide VB wherein PG represents a protecting group of the carboxylic acid function such as methyl ester, ethyl ester, etc. From intermediate VB, among existing chemical reactions, there may be mentioned Friedel-Crafts acylation providing VIB, formylation reaction providing VIB′, or halogenation reaction providing VIB″. In one embodiment, intermediate VIB can be transformed through Baeyer-Villiger rearrangement which can further provide, after hydrolysis, hydroxylated analogues, ether analogues or aryl derivatives. Finally, group R₄ is introduced according to General Schemes 5 or 6. In one another embodiment, intermediate VIB′ can be transformed through a reduction reaction to provide hydroxymethyl analogues. Finally, group R₄ is introduced according to General Schemes 5 or 6. In one another embodiment, intermediate VIB″ can be transformed through an alkylation reaction on the benzene ring. Finally, group R₄ is introduced according to General Schemes 5 or 6.

It is understood that IVA, IVD, VA, VA′, VB, VIA, VIB, VIB′, VIB″, VIIA and VIIA′ can represent particular compounds of Formula (I) or can represent intermediates for the preparation of compounds of Formula (I). For example, as shown in General Scheme 8, transformation of carboxylic acid of VIIA can be performed for the preparation of VIIIA or for the preparation of IXA.

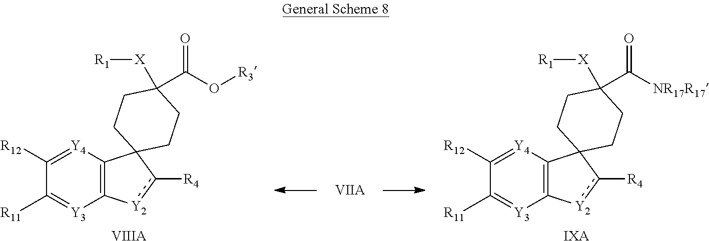

wherein X, Y₂, Y₃, Y₄, R₁, R₄, R₁₁, R₁₂, R₁₇, R₁₇′ and [symbol] are as defined in Formula (I), and R₃′ represents a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)(OR_{3A})₂, —W₁—SO₂—OR_{3A}, or —W₁—Cy₁.

A mixture of enantiomers, diastereoisomers resulting from the processes described above can be separated into their single components by chiral salt technique, chromatography using normal phase, reverse phase or chiral column, depending on the nature of the separation.

ABBREVIATIONS

| abbreviation | name |
|---|---|
| 2-Me-THF | 2-methyl-tetrahydrofuran |
| Ac | acetyl |
| AcCl | acetyl chloride |
| AcOH | acetic acid |
| AtaPhos | bis(di-tert-butyl(4- |
| | dimethylaminophenyl)phosphine)dichloropalladium(II) |
| aq. | aqueous |
| B₂pin₂ | bis(pinacolato)diboron |
| BBr₃ | boron tribromide |
| BF₃ × Et₂O | boron trifluoride diethyl etherate |
| BH₃ × SMe₂ | borane dimethyl sulfide complex |
| BH₃ × THF | borane tetrahydrofuran complex |
| Boc₂O | di-tert-butyl dicarbonate |
| Bn | benzyl |
| BnBr | benzyl bromide |
| BnOH | benzyl alcohol |
| cataCXium ® A | di(1-adamantyl)-n-butylphosphine |
| cc. | concentrated |
| CHCl₃ | chloroform |
| CuI | copper (I) iodide |
| Cu(OAc)₂ | copper(II) acetate |
| Cu(OTf)₂ | copper(II) trifluoromethanesulfonate |
| DAST | diethylaminosulfur trifluoride |
| DBU | 1,8-diazabicylco[5.4.0]undec-7-ene |
| DCM | methylene chloride |
| DDQ | 4,5-dichloro-3,6-dioxo-cyclohexa-1,4-diene-1,2-dicarbonitrile |
| DEA | diethylamine |
| DIAD | diisopropyl azodicarboxylate |
| DIBAL-H | diisobutyl aluminium hydride |
| DIPA | diisopropylamine |
| DIPE | diisopropyl ether |
| DIPEA | diisopropylethylamine |
| DMA | N,N-dimethylacetamide |
| DMAP | 4-dimethylaminopyridine |
| DME | 1,2-dimethoxyethane |
| DMF | N,N-dimethylformamide |
| DMP | Dess-Martin periodinane |
| DMSO | dimethyl sulfoxide |
| dppp | 1,3-bis(diphenylphosphino)propane |
| DTBAD | di-tert-butyl azodicarboxylate |
| eq. | equivalent |
| EDC × HCl | N-(3-dimethylaminopropyl)-N′-ethylcarbodiimide hydrochloride |
| Et | ethyl |
| Et₂O | diethyl ether |
| EtI | iodoethane |
| EtMgCl | ethyl magnesium chloride |
| EtOAc | ethyl acetate |
| EtOH | ethanol |
| EtSH | ethanethiol |
| h | hour(s) |
| HBTU | 2-(1H-benzotriazole-1-yl)-1,1,3,3-tetramethylaminium |
| | hexafluorophosphate |
| Herrmann's catalyst | trans-bis(acetato)bis[o-(di-o- |
| | tolylphosphino)benzyl]dipalladium(II) |
| HOBt | hydroxybenzotriazole |
| iPrMgCl | isopropyl magnesium chloride |
| iPrOH/IPA | isopropyl alcohol |
| Josiphos SL-J009 | (R)-1-[(SP)-2-(dicyclohexylphosphino) ferrocenyl]ethyldi-tert- |
| | butylphosphine |
| KOAc | potassium acetate |
| KOtBu | potassium tert-butoxide |
| LAH | lithium aluminiumhydride |
| LDA | lithium diisopropylamide |
| LiHMDS | [bis(trimethylsilyl)amino]lithium |
| mCPBA | 3-chloroperoxybenzoic acid |
| Me | methyl |
| MeCN | acetonitrile |
| MeI | iodomethane |
| MeLi | methyl lithium |
| MeMgCl | methyl magnesium chloride |
| MeMgBr | methyl magnesium bromide |
| MeOH | methanol |
| MeReO₃ | methyltrioxorhenium (VII) |
| MgSO₄ | magnesium sulfate |
| min | minute(s) |
| MnO₂ | manganese (IV) oxide |
| MOM-Cl | chloromethyl methyl ether |
| MsCl | methanesulphonyl chloride |
| MVK | methyl vinyl ketone |
| NaBH₄ | sodium borohydride |
| NaCN | sodium cyanide |
| NaH | sodium hydride |
| NaHCO₃ | sodium bicarbonate |
| NaOMe | sodium methoxide |
| Na₂SO₄ | sodium sulfate |
| NBS | N-bromosuccinimide |
| nBuLi | n-butyl lithium |
| nPrOH | propanol |
| NCS | N-chlorosuccinimide |
| NFSI | N-fluorobis(phenylsulfonyl)amine |
| NIS | N-iodosuccinimide |
| NH₃ | ammonia |
| NH₄Cl | ammonium chloride |
| NH₄HCO₃ | ammonium bicarbonate |
| NH₄HCO₂ | ammonium formate |
| NiCl₂ × glyme | nickel(II)chloride ethylene glycol dimethyl ether complex |
| Ni(dppp)Cl₂ | [1,3-bis(diphenylphosphino)propane]dichloronickel(II) |
| Pd/C | palladium on activated charcoal |
| Pd(dppf)Cl₂ | [1,1′-bis(diphenylphosphino)ferrocene] dichloropalladium (II) |
| Pd(dppf)Cl₂ × DCM | [1,1′-bis(diphenylphosphino)ferrocene] dichloropalladium (II), |
| | complex with dichloromethane |
| Pd₂(dba)₃ | tris(dibenzylideneacetone)dipalladium (0) |
| Pd(OAc)₂ | palladium (II) acetate |
| Pd(PPh₃)₂Cl₂ | bis(triphenylphosphine)palladium(II) dichloride |
| PE | petroleum ether |
| PhNTf₂ | bis(trifluoromethanesulfonyl)aniline |
| Ph₂O | diphenyl ether |
| PPh₃ | triphenylphosphine |
| PMB | 4-methoxybenzyl |
| PMB-Br | 4-methoxybenzyl bromide |
| PMB-Cl | 4-methoxybenzyl chloride |
| POCl₃ | phosphorus (V) oxychloride |
| PPA | polyphosphoric acid |
| PPTS | pyridinium p-toluenesulfonate |
| Pt/C | platinum on activated charcoal |
| PTFE | polytetrafluoroethylene |
| PtO₂ | platinum (IV) oxide |
| PTSA | p-toluenesulfonic acid monohydrate |
| Rh₂(OAc)₄ | rhodium(II) acetate dimer |
| rt | room temperature |
| RuPhos | 2-dicyclohexylphosphino-2′,6′-diisopropoxybiphenyl |
| RuPhos Pd G2 | chloro(2-dicyclohexylphosphino-2′,6′-diisopropoxy-1,1′- |
| | biphenyl)[2-(2′-amino-1,1′-biphenyl)]palladium(II) |
| sat. | saturated |
| SOCl₂ | thionyl chloride |
| STAB | sodium triacetoxyborohydride |
| TBAB | tetrabutyl ammonium bromide |
| TBACl | tetrabutyl ammonium chloride |
| TBAF | tetrabutyl ammonium fluoride |
| TBAI | tetrabutyl ammonium iodide |
| tBu | tert-butyl |
| tBuBr | tert-butylbromide |
| tBuOH | tert-butanol |
| BuXPhos | 2-di-tert-butylphosphino-2′,4′,6′-triisopropylbiphenyl |
| TBDMS-Cl | tert-butyldimethylsilyl chloride |
| TBDMS-OTf | tert-butyldimethylsilyl triflate |
| TBDPS-Cl | tert-butyldiphenylchlorosilane |
| TBTU | O-(benzotriazole-1-yl)-N,N,N′,N′-tetramethyluronium |
| | tetrafluoroborate |
| TEA | triethylamine |
| TFA | trifluoroacetic acid |
| TFAA | trifluoroacetic acid anhydride |
| Tf₂O | trifluoromethanesulphonic anhydride |
| THF | tetrahydrofuran |
| TMSCHNN | diazomethyl(trimethyl)silane |
| TMS-Cl | trimethylchorosilane |
| TMS-CN | trimethylsilylcyanide |
| TsCl | para-toluenesulfonyl chloride |
| Urotropin | 1,3,5,7-tetrazatricyclo[3.3.1.13,7]decane |

### General Synthetic Remarks

All reagents obtained from commercial sources were used without further purification. Anhydrous solvents were obtained from commercial sources and used without further drying. The reactions were monitored using LCMS and GCMS instruments and/or TLC.

Thin layer chromatography was conducted with 5 cm×10 cm plates coated with Merck Type 60 F₂₅₄ silica-gel.

Analytical LC-MS: The compounds of the present invention were characterized by high performance liquid chromatography-mass spectroscopy (HPLC-MS) using the following instruments:
Agilent HP1200 LC with Agilent MSD 6140 single quadrupole, operating in positive or negative ion electrospray ionisation mode. Molecular weight scan range is 100 to 1350 m/z. Parallel UV detection was done at 210 nm and 254 nm. Samples were supplied as a 1 mM solution in MeCN, or in THF/water (1:1) with 5 μL loop injection. LCMS analyses were performed on 2 instruments, one of which was operated with basic, and the other with acidic eluents.
Basic LCMS: Gemini-NX, 3 μm, C18, 50 mm×3.00 mm i.d. column at 23° C., at a flow rate of 1 mL min⁻¹ using 5 mM aq. NH₄HCO₃ solution (Solvent A) and MeCN (Solvent B) with a gradient starting from 100% Solvent A and finishing at 100% Solvent B over various duration of time.
Acidic LCMS: ZORBAX Eclipse XDB-C18, 1.8 μm, 50 mm×4.6 mm i.d. column at 40° C., at a flow rate of 1 mL min⁻¹ using 0.02% V/V aq. HCOOH solution (Solvent A) and 0.02% V/V HCOOH solution in MeCN (Solvent B) with a gradient starting from 100% Solvent A and finishing at 100% Solvent B over various duration of time.
Agilent 1200 SL series instrument linked to an Agilent MSD 6140 single quadrupole with an ESI-APCI multimode source or using an Agilent 1290 Infinity II series instrument connected to an Agilent TOF 6230 with an ESI-jet stream source; column: Thermo Accucore 2.6 μm, C18, 50 mm×2.1 mm at 55° C. or Agilent Zorbax Eclipse plus 3.5 μm, C18, 30 mm×2.1 mm at 35° C.; eluents: Solvent A: 10 mM aq. NH₄OAc solution+0.08% (v/v) HCOOH; Solvent B: MeCN+5% (v/v) Solvent A+0.08% (v/v) HCCOH, with a gradient starting from 95% Solvent A and finishing at 95% Solvent B or starting from 60% Solvent A and finishing at 98% Solvent B over various duration of time; ionisation is recorded in positive mode, negative mode, or positive-negative switching mode.

Combination gas chromatography and low-resolution mass spectrometry (LRMS) were performed on Agilent 6850 gas chromatograph and Agilent 5975C mass spectrometer using 15 m×0.25 mm column with 0.25 μm HP-5MS coating and helium as carrier gas. Ion source: EI⁺, 70 eV, 230° C., quadrupole: 150° C., interface: 300° C.

Microwave heating was performed in an Anton Parr MonoWave or CEM Discover® instrument.

Flash chromatography was performed on ISCO CombiFlash Rf 200, Rf 200i and Rf+ Lumen™ with pre-packed silica-gel cartridges (RediSep® Rf Normal-phase Silica Flash Columns (35-70 μm, 60 Å), RediSep Rf Gold© Normal-phase Silica High Performance Columns (20-40 μm, 60 Å), RediSep® Rf Reversed-phase C18 Columns (40-63 μm, 60 Å), or RediSep Rf Gold© Reversed-phase C18 High Performance Columns (20-40 μm, 100 Å).

Preparative HPLC purifications were performed on the following instruments:
1. Armen Spot Liquid Chromatography system with a Gemini-NX© 10 μM C18, 250 mm×50 mm i.d. column running at a flow rate of 118 mL min⁻¹ with UV diode array detection (210-400 nm) using 25 mM aq. NH₄HCO₃ solution and MeCN as eluents unless specified otherwise.
2. CombiFlash EZ Prep (Teledyne ISCO) system with a Gemini-NX© 10 μM C18, 250 mm×50 mm i.d. column running at a flow rate of 118 mL min⁻¹ with UV detection (210-400 nm) using 25 mM aq. NH₄HCO₃ solution and MeCN as eluents unless specified otherwise.
3. Waters FractionLynx MS autopurification system, with a Gemini® 5 μm C18(2), 100 mm×20 mm i.d. column from Phenomenex, running at a flow rate of 20 mL min⁻¹ with UV diode array detection (210-400 nm) and mass-directed collection. The mass spectrometer was a Waters Micromass ZQ2000 spectrometer, operating in positive or negative ion electrospray ionisation modes, with a molecular weight scan range of 150 to 1000. pH4 eluents: Solvent A: 10 mM aq. NH₄OAc solution+0.08% (v/v) HCOOH; Solvent B: MeCN+5% (v/v) Solvent A+0.08% (v/v) HCCOH. pH9 eluents: Solvent A: 10 mM aq. NH₄OAc solution+0.08% (v/v) cc. aq. NH₃ solution; Solvent B: MeCN+5% (v/v) Solvent A+0.08% (v/v) cc. aq. NH₃ solution.
4. AccQPrep HP125 (Teledyne ISCO) system, with a Gemini® NX 5 μm C18(2), 150 mm×21.2 mm i.d. column from Phenomenex, running at a flow rate of 20 mL min⁻¹ or Gemini® NX 5 μm C18(2), 250 mm×30 mm i.d. column from Phenomenex, running at a flow rate of 40 mL min⁻¹ with UV (214 and 254 nm) and ELS detection. pH4 eluents: Solvent A: water+0.08% (v/v) HCOOH; solvent B: MeCN+0.08% (v/v) HCOOH. pH9 eluents: Solvent A: water+0.08% (v/v) cc. aq. NH₃ solution; solvent B: MeCN+0.08% (v/v) cc. aq. NH₃ solution. Neutral eluents: Solvent A: water; Solvent B: MeCN.

¹H-NMR measurements were performed on Bruker Avance III 500 MHz spectrometer, Bruker Avance III 400 MHz spectrometer, Bruker DPX 400 MHz spectrometer and Bruker Avance NEO 400 MHz spectrometer, using DMSO-d₆ or CDCl₃ as solvent. ¹H NMR data is in the form of delta values, given in part per million (ppm), using the residual peak of the solvent (2.50 ppm for DMSO-d₆ and 7.26 ppm for CDCl₃) as internal standard. Splitting patterns are designated as: s (singlet), d (doublet), t (triplet), q (quartet), quint (quintet), sept (septet), m (multiplet), br (broad), br s (broad singlet), br d (broad doublet), br t (broad triplet), br m (broad multiplet), dd (doublet of doublets), td (triplet of doublets), dt (doublet of triplets), tt (triplet of triplets), tm (triplet of multiplets), qd (quartet of doublets), ddd (doublet of doublet of doublets), dm (doublet of multiplets).

HRMS were determined on a Shimadzu IT-TOF, ion source temperature 200° C., ESI+/−, ionization voltage: (+−)4.5 kV. Mass resolution min. 10000.

Chemical names were generated using ACD/Labs 2019.1.2. (File version: C05H41, Build: 111302, 27 Aug. 2019).

### General Procedures

### General Procedure 1: LAH Reduction of Esters

LAH (3 eq.) was added portionwise to dry THE (2 mL/mmol ester) under N₂ atmosphere. The mixture was stirred at 40-50° C. for 15 min. Then the appropriate ester (1 eq.) in dry THE (1 mL/mmol ester) was added dropwise while maintaining the temperature between 55 and 60° C. The mixture was stirred at reflux temperature for 3 h, then it was allowed to cool to rt and stirred overnight. The reaction mixture was cooled to 0° C. Water (2 mL/g LAH) was added dropwise, followed by the dropwise addition of 15% aq. NaOH solution (2 mL/g LAH) at 0-10° C. The mixture was stirred for 15 min, then water (6 mL/g LAH) was added and the mixture was stirred at rt for 1 h. The precipitate was filtered off. The filtrate was concentrated under reduced pressure, then DCM and water were added. The layers were separated and the aq. layer was extracted with DCM. The combined organic layers were dried over Na₂SO₄, filtered and the filtrate was concentrated under reduced pressure. The crude intermediate was used without further purification.

### General Procedure 2: Monoprotection of the Diols

The appropriate diol (1 eq.) was dissolved in MeCN (3 mL/mmol diol), then KI (1 eq.) and K₂CO₃ (1.1 eq.) were added, followed by the addition of 2-diphenylboranyloxyethanamine (0.08 eq.). The mixture was stirred for 5 min, then PMB-Cl (1.5 eq.) was added and the mixture was stirred at 60° C. until no further conversion was observed. The mixture was allowed to cool to rt, diluted with EtOAc and washed with water. The layers were separated and the aq. layer was extracted with EtOAc. The combined organic layers were dried over Na₂SO₄, filtered and the filtrate was concentrated under reduced pressure. The crude intermediate was purified via flash chromatography using heptane and EtOAc as eluents.

### General Procedure 3: Bromination of Alcohols

PPh₃ (1.1 eq.) was dissolved in DCM (0.25 mL/mmol alcohol) and cooled to 0° C. Br₂ (1.2 eq.) dissolved in DCM (0.25 mL/mmol alcohol) was added dropwise. The mixture was stirred at rt for 1 h then it was cooled to 0° C. The mixture of the appropriate alcohol (1 eq.) and TEA (1.25 eq.) in DCM (1.5 mL/mmol alcohol) was added dropwise at 0° C. After stirring at 0° C. for 30 min the mixture was allowed to warm to rt and stirred overnight. Then it was quenched with sat. aq. Na₂S₂O₃ solution and water, the layers were separated, the aq. layer was extracted with DCM. The combined organic layers were dried over Na₂SO₄, filtered and the filtrate was concentrated under reduced pressure. Heptane was added, and the mixture was stirred and sonicated. The precipitate was filtered and washed with heptane. The filtrate was concentrated under reduced pressure and purified via flash chromatography using heptane and EtOAc as eluents.

### General Procedure 4: Zn Reagent Formation

To an oven-dried flask zinc (1.4 eq.) was added and the vessel was heated at 160° C. for 1 h under vacuum then allowed to cool to rt and placed under N₂ atmosphere. DMA (0.7 mL/mmol bromo compound) was added followed by the addition of I₂ (0.05 eq.). The mixture was stirred at rt for 5 min, then the appropriate bromo compound (1 eq.) in DMA (0.6 mL/mmol bromo compound) was added and the mixture was stirred at 75° C. for 18 h, then it was allowed to cool to rt. Cannulation through a filter (cotton-wool/Celite/cotton-wool) into a dry Schlenk tube afforded the desired product as a solution (concentration determined by titration with a 0.5 M solution of 12) that was used without further characterization.

### General Procedure 5: Bromination of Indan-1-Ones

The appropriate indan-1-one (1 eq.) was dissolved in DCM (1.5 mL/mmol indan-1-one), then NBS (1.1 eq.) and PTSA (0.1 eq.) were added at rt. The mixture was stirred at reflux temperature until no further conversion was observed. The mixture was allowed to cool to rt. It was quenched with water and brine and extracted with DCM. The combined organic layers were dried over Na₂SO₄, filtered and the filtrate was concentrated under reduced pressure. The crude intermediate was purified via flash chromatography using heptane and EtOAc as eluents.

### General Procedure 6: Oxo Reduction of Bromo-Indan-1-Ones

The appropriate bromo-indan-1-one (1 eq.) was dissolved in DCM or MeOH (3.5 mL/mmol bromo-indan-1-one), cooled to 0° C., then NaBH₄ (1-2 eq.) was added portionwise. The mixture was stirred at rt until no further conversion was observed. The mixture was quenched with water and brine. The layers were separated and the aq. layer was extracted with DCM. The combined organic layers were dried over Na₂SO₄, filtered and the filtrate was concentrated under reduced pressure. The crude intermediate was used without further purification.

General Procedure 7: Water Elimination from Indanes

The appropriate indane (1 eq.) was dissolved in toluene (50 mL/mmol indane) in a flask equipped with a Dean Stark apparatus. PTSA (0.64 eq.) was added and the mixture was stirred at reflux temperature until no further conversion was observed. The mixture was allowed to cool to rt. Sat. aq. NaHCO₃ solution was added and the layers were separated. The organic layer was washed with brine, dried over Na₂SO₄, filtered and the filtrate was concentrated under reduced pressure. The crude intermediate was purified via flash chromatography using heptane and DCM or heptane and EtOAc as eluents.

General Procedure 8a: Spirocyclization with NaH in DMF

The appropriate indene (1 eq.) and Preparation 1b (or Preparation 1a, where noted, 1.1 eq.) were dissolved in dry DMF (4 mL/mmol indene) and cooled to 0° C. under N₂ atmosphere. NaH (2.2 eq., 60% dispersion in mineral oil) was added. After stirring for 1 h at 0° C., the mixture was allowed to warm to rt, and stirred until no further conversion was observed. Then it was quenched with sat. aq. NH₄Cl solution and stirred for 30 min. Sat. aq. NaHCO₃ solution was added and the mixture was extracted with EtOAc. The combined organic layers were dried over Na₂SO₄, filtered and the filtrate was concentrated under reduced pressure. The crude intermediate was purified via flash chromatography using heptane and EtOAc or DCM and MeOH as eluents.

General Procedure 8b: Spirocyclization with LiHMDS in THE

The appropriate indene or isoindolin-1-one (1 eq.) was dissolved in dry THE (10 mL/mmol indene or isoindolin-1-one) and cooled to −78° C. under N₂ atmosphere. LiHMDS (1 M solution in THF, 2.2 eq.) was added, and the mixture was stirred at −78° C. for 30 min under N₂ atmosphere. Preparation 1b (1.2 eq.) was dissolved in dry THE (1 mL/mmol indene or isoindolin-1-one) and was added dropwise at −78° C. Then it was allowed to warm to rt and stirred until no further conversion was observed. Then it was quenched with sat. aq. NH₄Cl solution and extracted with EtOAc. The combined organic layers were washed with brine, dried over Na₂SO₄, filtered and the filtrate was concentrated under reduced pressure. The crude intermediate was purified via flash chromatography using heptane and EtOAc as eluents.

### General Procedure 9: Ketal Cleavage

The appropriate ketal (or acetal, 1 eq.) was dissolved in acetone (6.2 mL/mmol ketal), then 2 M aq. HCl solution (4.4 mL/mmol ketal) was added. The mixture was stirred at 45° C. until no further conversion was observed. Then it was allowed to cool to rt. The pH was adjusted to 7 with sat. aq. NaHCO₃ solution and acetone was removed under reduced pressure. Then it was extracted with EtOAc. The combined organic layers were dried over Na₂SO₄, filtered and the filtrate was concentrated under reduced pressure. The crude intermediate was purified via flash chromatography using heptane and EtOAc as eluents or DCM and MeOH (1.2% NH₃) as eluents.

General Procedure 10: Bargellini Reaction with Ketones

The appropriate ketone (1 eq.) was dissolved in THE (5 mL/mmol ketone), then it was cooled to 0° C. The appropriate aniline or phenol or thiol (1-3 eq.) and solid NaOH (3-5 eq.) were added, followed by the dropwise addition of CHCl₃ (3-5 eq.). The mixture was stirred at 0° C. for 30 min, then it was allowed to warm to rt and it was stirred at rt until no further conversion was observed. Then it was quenched with water, and the pH was adjusted to 3-4 with 2 M aq. HCl solution. Then it was extracted with EtOAc. The combined organic layers were dried over Na₂SO₄, filtered and the filtrate was concentrated under reduced pressure. The crude intermediate was purified via flash chromatography using heptane and EtOAc as eluents or via prep RP-HPLC using 25 mM aq. NH₄HCO₃ solution and MeCN as eluents.

General Procedure 11: Strecker Reaction with Ketones

The appropriate ketone (1 eq.) and the appropriate aniline or pyridine-amine (1.2 eq.) were dissolved in AcOH (10 mL/mmol ketone), then TMS-CN (1.2 eq.) was added dropwise. The mixture was stirred at rt until no further conversion was observed. The pH was adjusted to 10 with 25% aq. NH₃ solution. Then it was extracted with EtOAc. The combined organic layers were dried over Na₂SO₄, filtered and the filtrate was concentrated under reduced pressure. The crude product was purified via flash chromatography using heptane and EtOAc as eluents.

General Procedure 12a: Hydrolysis of Nitriles with Acetaldoxime/InCl₃

The appropriate nitrile (1 eq.) was dissolved in dry toluene (4 mL/mmol nitrile), then acetaldoxime (4.5 eq.) and InCl₃ (0.07 eq.) were added. The mixture was stirred at 75° C. until no further conversion was observed. The mixture was allowed to cool to rt. Toluene was removed under reduced pressure. The crude intermediate was purified via flash chromatography using heptane and EtOAc or EtOAc and MeOH or DCM and MeOH as eluents.

General Procedure 12b: Hydrolysis of Nitriles with H₂O₂

The appropriate nitrile (1 eq.) was dissolved in MeOH (70 mL/mmol nitrile), then 1 M aq. NaOH solution (5 mL/mmol nitrile) was added. H₂O₂ solution (30%, 10 mL/mmol nitrile) was added at 10° C. in portions. After 30 min stirring at rt, the mixture was heated to 35° C.-50° C. and stirred until no further conversion was observed. Sat. aq. Na₂S₂O₃ solution was added under cooling, then MeOH was removed under reduced pressure. Water was added and it was extracted with EtOAc. The combined organic layers were dried over Na₂SO₄, filtered and the filtrate was concentrated under reduced pressure. The crude intermediate was purified via flash chromatography using heptane and EtOAc as eluents.

### General Procedure 13: Hydrolysis of Amides

The appropriate amide (1 eq.) was dissolved in 2-methoxy-ethanol (8 mL/mmol amide), then NaOH (15 eq.) and water (0.8 mL/mmol amide) were added. The mixture was stirred at 120° C.-200° C. with or without microwave irradiation until no further conversion was observed. The mixture was allowed to cool to rt. The pH was set to 2-3 with 2 M aq. HCl solution. The mixture was extracted with EtOAc and the combined organic layers were dried over Na₂SO₄, filtered and the filtrate was concentrated under reduced pressure. The crude intermediate was purified via flash chromatography using heptane and EtOAc or DCM and MeOH as eluents or via prep RP-HPLC using 25 mM aq. NH₄HCO₃ solution and MeCN as eluents.

General Procedure 14: Bucherer-Bergs Reaction with Ketones

A flask was charged with the appropriate ketone (1 eq.), (NH₄)₂CO₃ (4 eq.), KCN (or NaCN where noted, 2 eq.), EtOH (5 mL/mmol ketone) and water (6 mL/mmol ketone). The mixture was stirred at 60° C. until no further conversion was observed. The mixture was allowed to cool to rt. A mixture of water and ice was added and it was stirred for 15 min. The precipitation was filtered, washed with water and dried under reduced pressure.

### General Procedure 15: Hydrolysis of Hydantoins

A teflon flask was charged with the appropriate hydantoin (1 eq.), then LiOH×H₂O (10 eq.) and water (3 mL/mmol hydantoin) were added. The mixture was stirred in an oil bath heated to 140° C. until no further conversion was observed. Then it was allowed to cool to rt. The pH was set to 7 with cc. aq. HCl solution. The formed precipitation was filtered, washed with water and dried under reduced pressure.

### General Procedure 16: Ullmann Coupling

A flask was charged with the appropriate amino acid (1 eq.), the appropriate iodobenzene or bromobenzene (1.2 eq.), CuI (0.1 eq.), ethyl-2-oxocyclohexanecarboxylate (0.4 eq.), Cs₂CO₃ (2 eq.) and DMF (10 mL/mmol amino acid) under N₂ atmosphere. The mixture was stirred at 105° C. until no further conversion was observed. Then it was allowed to cool to rt. DMF was removed under reduced pressure. Water and brine were added and it was extracted with DCM or EtOAc. The combined organic layers were dried over Na₂SO₄, filtered and the filtrate was concentrated under reduced pressure. The crude intermediate was purified via flash chromatography using heptane and EtOAc as eluents or via prep RP-HPLC using 25 mM aq. NH₄HCO₃ solution and MeCN as eluents.

General Procedure 17a: Esterification of Acids with TMS-CHNN

The appropriate amino acid (1 eq.) was dissolved in DCM (5 mL/mmol amino acid) and MeOH (5 mL/mmol amino acid), then TMS-CHNN (2-4 eq.) was added. The mixture was stirred at rt until no further conversion was observed. The solvents were removed under reduced pressure and the crude intermediate was purified via flash chromatography using heptane and EtOAc or DCM and MeOH as eluents.

General Procedure 17b: Esterification of Acids with MeI

The appropriate amino acid (1 eq.) was dissolved in DMF (8 mL/mmol amino acid), then cooled to 0° C. Cs₂CO₃ (1 eq.) and MeI (1.3 eq.) were added. The mixture was stirred at 0° C. until no further conversion was observed. DMF was removed under reduced pressure, then water and brine were added and it was extracted with EtOAc. The combined organic layers were dried over Na₂SO₄, filtered and the filtrate was concentrated under reduced pressure. The crude intermediate was purified via flash chromatography using heptane and EtOAc as eluents.

General Procedure 18: Suzuki Coupling with 2-Bromo-Indenes

A microwave vial was charged with the appropriate 2-bromo-indene derivative (1 eq.), the appropriate boronic acid or ester (1.5-3 eq.), Cs₂CO₃ (3 eq.) and 1,4-dioxane (10 mL/mmol indene) and water (3 mL/mmol indene). The vial was purged with N₂, followed by the addition of Pd(PPh₃)₄ (0.1 eq.). The mixture was heated at 120° C. for 30 min under microwave irradiation. Then it was diluted with water, the pH was set to 3 with 2 M aq. HCl solution. It was extracted with EtOAc. The combined organic layers were dried over Na₂SO₄, filtered and the filtrate was concentrated under reduced pressure. The crude product was purified via flash chromatography using heptane and EtOAc as eluents or via prep RP-HPLC using 25 mM aq. NH₄HCO₃ solution and MeCN as eluents.

### General Procedure 19: Hydrogenation of Indenes

The appropriate indene (1 eq.) was dissolved in EtOAc (15 mL/mmol indene). 10% Pt/C (0.1 g catalyst/g indene) was added and the flask was evacuated and backfilled with N₂ (×3), then evacuated and filled with H₂. Then the mixture was stirred at rt until no further conversion was observed. Then it was filtered, washed with EtOAc, and the filtrate was concentrated under reduced pressure. When the reduction stopped at low conversion, the hydrogenation procedure was repeated using fresh catalyst. The crude product was purified via flash chromatography using heptane and EtOAc as eluents or via prep RP-HPLC using 25 mM aq. NH₄HCO₃ solution and MeCN as eluents.

General Procedure 20: Coupling with NaAuCl₄

Preparation 7b (1 eq.), the appropriate alcohol (5 eq.) and NaAuCl₄×2H₂O (0.05 eq.) were measured into a vial, sealed and stirred at 70° C. until no further conversion was observed. Then the mixture was diluted with DCM and MeOH, filtered and the filtrate was concentrated. The crude product was purified via flash chromatography using heptane and EtOAc as eluents, and then via prep RP-HPLC using 25 mM aq. NH₄HCO₃ solution and MeCN as eluents.

General Procedure 21: Amide Formation from Carboxylates

The appropriate carboxylic acid (1 eq.) was dissolved in pyridine (12 mL/mmol carboxylic acid). The appropriate amine (1.1 eq.) and EDC×HCl (3 eq.) were added and the mixture was stirred at rt under N₂ atmosphere until no further conversion was observed. Then it was concentrated under reduced pressure and purified via flash chromatography using heptane and EtOAc as eluents.

### General Procedure 22: TFA Amide Formation

The appropriate indene (1 eq.) was dissolved in 2-Me-THF (2.25 mL/mmol indene), then TEA (5 eq.) and DMAP (0.1 eq.) were added, then cooled to 0° C. TFAA (20 eq.) was added dropwise at 0° C. (keeping the temperature of the mixture below 10° C.), then it was stirred at 50° C. until no further conversion was observed. Then it was cooled to 0° C. and stirred for 2 h. The precipitate was filtered, taken up in DIPE and sonicated. The precipitate was filtered, washed with DIPE and dried.

### General Procedure 23: Friedel Crafts Acylation of Indenes

To a suspension of AlCl₃ (3 eq.) in DCM (6 mL/mmol indene) a solution of AcCl (2 eq.) in DCM (2 mL/mmol indene) was added at 0° C. under N₂ atmosphere and the mixture was stirred at 0° C. for 30 min. Then a solution of the appropriate indene (1 eq.) in DCM (2 mL/mmol indene) was added dropwise and the mixture was stirred at 0° C. until no further conversion was observed. Then it was poured onto ice-water and stirred for 15 min. The layers were separated and the aq. layer was extracted with DCM. The combined organic layers were dried over Na₂SO₄, filtered and the filtrate was concentrated under reduced pressure. The crude product was purified via flash chromatography using heptane and EtOAc as eluents.

### General Procedure 24: Baeyer-Villiger Oxidation of Acetyl-Indenes

The appropriate indene (1 eq.) was dissolved in DCM (10 mL/mmol), then Na₂HPO₄ (10 eq.) and mCPBA (2.05 eq.) were added. The mixture was stirred at rt until no further conversion was observed. Then it was diluted with water, and stirred for 20 min. The precipitation was filtered, and the filtrate was extracted with DCM. The combined organic layers were washed with water, dried over Na₂SO₄, filtered and the filtrate was concentrated under reduced pressure. The crude product was purified via flash chromatography using heptane and EtOAc as eluents.

### General Procedure 25: Acetyl Cleavage of Acetoxy-Indenes

The appropriate indene (1 eq.) was dissolved in MeOH (6 mL/mmol indene), then NaOMe (1.65 eq.) was added under N₂ atmosphere. The mixture was stirred at rt until no further conversion was observed. Then it was diluted with water, the pH was set to 6 with 2 M aq. HCl solution and it was extracted with DCM. The combined organic layers were dried over Na₂SO₄, filtered and the filtrate was concentrated under reduced pressure. The crude product was purified via flash chromatography using heptane and EtOAc as eluents.

### General Procedure 26: Formylation of Indenes

The appropriate indene (1 eq.) was dissolved in TFA (5 mL/mmol indene), then urotropine (3 eq.) was added. The mixture was stirred at reflux temperature until no further conversion was observed. The reaction mixture was concentrated under reduced pressure and purified via flash chromatography using heptane and EtOAc as eluents.

General Procedure 27a: Negishi Coupling with AtaPhos

An oven-dried round-bottom flask, equipped with a PTFE-coated magnetic stirring bar was charged with the appropriate 2-bromo-indene derivative (1 eq.) and AtaPhos (0.02 eq.), then dry THE (6 mL/mmol indene) was added under N₂ atmosphere. 1-Methylimidazole (1.7 eq.) and the appropriate Zn reagent (2 eq.) were added and the mixture was stirred at 50° C. or at 120° C. under microwave irradiation until no further conversion was observed. The mixture was allowed to cool to rt, then diluted with sat. aq. NH₄Cl solution and water, then extracted with EtOAc. The combined organic layers were washed with brine, then dried over Na₂SO₄, filtered and the filtrate was concentrated under reduced pressure. The crude intermediate was purified via flash chromatography using heptane and EtOAc as eluents or via prep RP-HPLC using 25 mM aq. NH₄HCO₃ solution and MeCN as eluents.

General Procedure 27b: Negishi Coupling with Pd(I)-I-Dimer

An oven-dried round-bottom flask, equipped with a PTFE-coated magnetic stirring bar was charged with the appropriate 2-bromo-indene derivative (1 eq.), then dry toluene (10 mL/mmol indene) was added under N₂ atmosphere. Di-μ-iodobis(tri-tert-butylphosphino)dipalladium (I) (0.02 eq.) was added under N₂ flow. The appropriate Zn reagent (1.25 eq.) was added at 50° C. and the mixture was stirred at 50-105° C. until no further conversion was observed. The mixture was allowed to cool to rt. The mixture was diluted with sat. aq. NH₄Cl solution and water, then filtered through a pad of Celite. The filtrate was extracted with EtOAc and the combined organic layers were washed with brine, then dried over Na₂SO₄, filtered and the filtrate was concentrated under reduced pressure. The crude intermediate was purified via flash chromatography using heptane and EtOAc as eluents.

General Procedure 28a: PMB Cleavage with DDQ

The appropriate PMB derivative (1 eq.) was dissolved in DCM (5 mL/mmol PMB derivative) and water (0.5 mL/mmol PMB derivative), then cooled to 0° C. DDQ (1.2 eq.) was added and the mixture was stirred at rt until no further conversion was observed. Then it was diluted with water and brine, then extracted with DCM. The combined organic layers were dried over Na₂SO₄, filtered and the filtrate was concentrated under reduced pressure. The crude intermediate was purified via flash chromatography using heptane and EtOAc as eluents.

General Procedure 28b: PMB Cleavage with TfOH

The appropriate PMB derivative (1 eq.) was dissolved in DCM (10 mL/mmol PMB derivative), then 1,3-dimethyoxybenzene (3 eq.) and TfOH (1.2 eq.) were added. The mixture was stirred at rt until no further conversion was observed. Then it was diluted with sat. aq. NaHCO₃ solution and water, then extracted with DCM. The combined organic layers were dried over Na₂SO₄, filtered and the filtrate was concentrated under reduced pressure. The crude intermediate was purified via flash chromatography using heptane and EtOAc or DCM and MeOH (1.2% NH₃) as eluents.

### General Procedure 29: Silyl Protecting Group Cleavage

The appropriate silyl derivative (1 eq.) was dissolved in THE (10 mL/mmol silyl derivative), then TBAF (1.1 eq.) was added. The mixture was stirred at rt until no further conversion was observed. Then it was diluted with sat. aq. NaHCO₃ solution and water, then extracted with EtOAc. The combined organic layers were dried over Na₂SO₄, filtered and the filtrate was concentrated under reduced pressure. The crude intermediate was purified via flash chromatography using heptane and EtOAc as eluents.

General Procedure 30a: Mitsunobu Coupling with DTABD

The appropriate indene or indane or isoindoline (1 eq.), PPh₃ (2-3 eq.) and the appropriate alcohol (2-3 eq.) were dissolved in THE or in toluene (10 mL/mmol indane). DTBAD (2-3 eq.) was added and the mixture was stirred at 50° C. until no further conversion was observed. The solvent was removed under reduced pressure and the crude intermediate was purified via flash chromatography using heptane and EtOAc as or EtOAc and MeOH as eluents or via prep RP-HPLC using 25 mM aq. NH₄HCO₃ solution and MeCN as eluents.

General Procedure 30b: Mitsunobu Coupling with DIAD

To a solution of the appropriate alcohol (1.5 eq.) and PPh₃ (2-3 eq.) dissolved in THE (5-10 mL/mmol alcohol) was added DIAD (1.5 eq.) dropwise at 0° C. or rt and the resulting solution was stirred for 5 min. The appropriate indene or indane or isoindoline (1 eq.) in THF (10 mL/mmol) was added and the mixture stirred at rt until no further conversion was observed. Then it was diluted with DCM, washed with sat. aq. NaHCO₃ solution and the organic phase was dried (PTFE phase separator) and concentrated in vacuo. The crude intermediate was purified via flash chromatography using heptane and EtOAc or MeOH and DCM as eluents.

General Procedure 31a: Coupling of Aryl Chlorides with Josiphos

The appropriate alcohol (1 eq.) was dissolved in toluene (5-10 mL/mmol alcohol), then Josiphos SL-J009 (0.1 eq.), the appropriate aryl chloride (1.2 eq.), Cs₂CO₃ (3 eq.) and allylpalladium(II) chloride dimer (0.05 eq.) were added. The mixture was sparged with N₂ and stirred at 90° C. until no further conversion was observed. The mixture was allowed to cool to rt. The mixture was partitioned between DCM and sat. aq. NaHCO₃ solution and the organic phase was washed with brine, dried (PTFE phase separator) and concentrated in vacuo. The crude intermediate was purified via flash chromatography using heptane and EtOAc or MeOH and DCM as eluents.

### General Procedure 31b: Alkylation of Aryl Chlorides

The appropriate alcohol (1 eq.) was dissolved in DMF (10 mL/mmol alcohol), then NaH (60% dispersion; 3 eq.) was added portionwise and the mixture was allowed to stir for 5 min at 0° C. or rt. The appropriate aryl chloride (1.5-2 eq.) in DMF (10 mL/mmol alcohol) was added. The mixture was stirred at 90° C. until no further conversion was observed, then it was allowed to cool to rt. It was quenched with water, then extracted with DCM. The combined organic extracts were washed with 1 M aq. HCl solution, brine, dried (PTFE phase separator) and concentrated in vacuo. The crude intermediate was purified via flash chromatography using heptane and EtOAc or MeOH and DCM as eluents or via prep RP-HPLC using MeCN and water as eluents.

### General Procedure 31c: Alkylation of Aryl Chlorides

To a solution of the appropriate alcohol (1 eq.) dissolved in DMSO (6 mL/mmol alcohol) was added KOtBu (3-4 eq.) and the mixture was stirred at rt for 5 min before the addition of the appropriate aryl chloride (1.3-2.3 eq.) in DMSO (2-5 mL/mmol aryl chloride). The mixture was stirred at rt or at 50° C. until no further conversion was observed. Then it was partitioned between DCM and water, and the organic phase was washed with 0.1 M aq. HCl solution, brine, dried (PTFE phase separator) and concentrated in vacuo. The crude intermediate was purified via flash chromatography using heptane and EtOAc or MeOH and DCM as eluents or via prep RP-HPLC using MeCN and water as eluents.

### General Procedure 32: Mitsunobu Coupling Followed by Hydrolysis

### Step A-Mistunobu Coupling

The appropriate indene or indane or isoindoline (1 eq.), PPh₃ (2-3 eq.) and the appropriate alcohol or amine (2-3 eq.) were dissolved in THE or in toluene (10 mL/mmol indane). DTBAD (2-3 eq.) was added and the mixture was stirred at 50° C. until no further conversion was observed. The solvent was removed under reduced pressure and the crude intermediate was purified via flash chromatography using heptane and EtOAc as eluents or via prep RP-HPLC using 25 mM aq. NH₄HCO₃ solution and MeCN as eluents.

### Step B-Hydrolysis

The obtained intermediate (1 eq.) was dissolved in 1,4-dioxane (10 mL/mmol ester), then water (10 mL/mmol ester) and LiOH×H₂O (10-20 eq.) were added and the mixture was stirred at 60° C. until no further conversion was observed. The mixture was allowed to cool to rt. The pH was set to 6-8 with 2 M aq. HCl solution, and then it was extracted with EtOAc. The combined organic layers were dried over Na₂SO₄, filtered and the filtrate was concentrated under reduced pressure. The crude product was purified via flash chromatography using heptane and EtOAc as eluents or via prep RP-HPLC using 25 mM aq. NH₄HCO₃ solution and MeCN as eluents.

### General Procedure 33a: Hydrolysis

The appropriate ester (1 eq.) was dissolved in 1,4-dioxane or MeOH (10 mL/mmol for the ester), then water (10 mL/mmol ester) and LiOH×H₂O (10-20 eq.) were added and the mixture was stirred at 60° C. until no further conversion was observed. The mixture was allowed to cool to rt. The pH was set to 6-8 with 2 M aq. HCl solution, and it was extracted with EtOAc. The combined organic layers were dried over Na₂SO₄, filtered and the filtrate was concentrated under reduced pressure. The crude product was purified via flash chromatography using heptane and EtOAc as eluents or via prep RP-HPLC using 25 mM aq. NH₄HCO₃ solution and MeCN as eluents.

### General Procedure 33b: Hydrolysis in Microwave Reactor

To the appropriate ester (1 eq.) dissolved in MeOH (10 mL/mmol for the ester) was added LiOH×H₂O (5-10 eq.) and the mixture was heated at 110-130° C. under microwave irradiation until no further conversion was observed. The mixture was allowed to cool to rt. Then it was diluted with water, acidified with 2 M aq. HCl solution and then extracted with DCM. The combined organic layers were dried over MgSO₄, filtered and the filtrate was concentrated under reduced pressure. The crude product was purified via flash chromatography using heptane and EtOAc, or MeOH and DCM as eluents or via prep RP-HPLC using MeCN and water as eluents.

General Procedure 34: Suzuki Coupling with Triflates

Preparation 16a (1 eq.), the appropriate boronic ester or acid (1.2-2 eq.), Cs₂CO₃ (2 eq.) and Pd(dppf)Cl₂ (0.1 eq.) were measured into a vial, the vial was purged with N₂. THE (5 mL/mmol triflate) and water (1.5 mL/mmol triflate) were added. The vial was sealed and the mixture was stirred at 85° C. until no further conversion was observed. Then the mixture was cooled to rt, and it was directly injected in the loop of the prep RP-HPLC and purified using 25 mM aq. NH₄HCO₃ solution and MeCN as eluents.

General Procedure 36: Carbonyl Reduction with NaBH₄

The appropriate acetyl or formyl derivative (1 eq.) was dissolved in MeOH (20 mL/mmol acetyl compound) and cooled to 0° C. NaBH₄ (2 eq.) was added portionwise, and the mixture was stirred at 0° C. until no further conversion was observed. Then it was quenched with sat. aq. NH₄Cl solution, and extracted with DCM. It was dried over Na₂SO₄, filtered and the filtrate was concentrated under reduced pressure. The crude intermediate was purified via flash chromatography using DCM and MeOH as eluents.

### General Procedure 37: Alkylation of Isoindolin-1-Ones

The appropriate isoindolin-1-one (1 eq.) was dissolved in dry DMF (2.5 mL/mmol isoindolin-1-one) and cooled to 0° C. under N₂ atmosphere. NaH (60% dispersion in mineral oil, 1.2 eq.) was added, then allowed to warm to rt and stirred at rt for 20 min. The appropriate alkyl bromide (1.5 eq.) was added dropwise and the mixture was stirred at rt until no further conversion was observed. Then it was quenched with sat. aq. NH₄Cl solution and extracted with EtOAc. The combined organic layers were washed with brine, dried over Na₂SO₄, filtered and the filtrate was concentrated under reduced pressure. The crude intermediate was purified via flash chromatography using heptane and EtOAc as eluents.

### General Procedure 38: Reduction of Isoindolin-1-Ones

The appropriate isoindolin-1-one (1 eq.) was dissolved in dry THE (3 mL/mmol isoindolin-1-one) and cooled to 0° C. under N₂ atmosphere. LAH in THE (1 M, 1.5-2.0 eq.) was added dropwise at 0° C. and the mixture was stirred at 0° C.-50° C. until no further conversion was observed. Then water (1 mL/g LAH) was added dropwise, followed by the dropwise addition of 15% aq. NaOH solution (1 mL/g LAH) at 0-10° C. The mixture was stirred for 15 min, then water (3 mL/g LAH) was added again and the mixture was stirred at rt for 1 h. Then it was filtered through a pad of Celite, washed with EtOAc. The filtrate was washed with brine, dried over Na₂SO₄, filtered and the filtrate was concentrated under reduced pressure. The crude product was purified via flash chromatography using heptane and EtOAc as eluents.

### General Procedure 39: Pd Catalysed Synthesis of Isoindolin-1-Ones

The appropriate benzamide (1 eq.) was dissolved in MeCN (10 mL/mmol benzamide). PPh₃ (0.5 eq.), K₂CO₃ (3 eq.), TBACl (1.5 eq.) and Pd(OAc)₂ (0.15 eq.) were added and the mixture was stirred under N₂ atmosphere at 80° C. until no further conversion was observed. Then it was diluted with sat. aq. NaHCO₃ solution, brine and extracted with EtOAc. The combined organic layers were washed with brine, dried over Na₂SO₄, filtered and the filtrate was concentrated under reduced pressure. The crude product was purified via flash chromatography using heptane and EtOAc as eluents.

General Procedure 40: Alkylation with Mesylates

To a solution of the appropriate mesylate (1 eq) in MeCN (10-20 mL/mmol) was added K₂CO₃ or Cs₂CO₃ (2-3 eq) and the appropriate alcohol or amine (1.2 eq). The mixture was heated at 70° C. until no further conversion was observed and then cooled to rt. The mixture was partitioned between DCM and water, and the organic phase was washed with brine, dried (MgSO₄) and concentrated in vacuo. The crude product was purified via flash chromatography using heptane and EtOAc or MeOH and DCM as eluents.

### General Procedure 41a: Silyl Protection of Alcohols, NaH Base

To a solution of the appropriate alcohol (1 eq.) in THE (5-10 mL/mmol) cooled to 0° C. under N₂ was added NaH (60% as a dispersion in mineral oil, 1-1.2 eq.) in portions and the mixture was stirred for 15 min. The appropriate silyl chloride (1-1.2 eq.) in THE (1-5 mL/mmol) was added dropwise, cooling removed, and the mixture was stirred at rt until no further conversion was observed. The mixture was quenched by the addition of sat. aq. NH₄Cl solution and partitioned between EtOAc and water. The organic phase was washed with brine, dried (MgSO₄), filtered and concentrated in vacuo. The crude material was purified via flash chromatography using heptane and EtOAc as eluents.

### General Procedure 41b: Silyl Protection of Alcohols, Imidazole Base

To a solution of the appropriate alcohol (1 eq.) and imidazole (2 eq.) in DMF (5-10 mL/mmol) the appropriate silyl chloride (1.1-1.4 eq.) was added dropwise and the mixture was stirred at rt until no further conversion was observed. The mixture was quenched by the addition of sat. aq. NH₄Cl solution and partitioned between EtOAc and water. The organic phase was washed with brine, dried (MgSO₄), filtered and concentrated in vacuo. The crude material was purified via flash chromatography using heptane and EtOAc as eluents.

### General Procedure 42: Tosyl Protection of Diols

To a solution of the appropriate alcohol (1 eq.) in DCM (1-2 mL/mmol) was added DMAP (0.1 eq), TEA (2.5-3.5 eq) and TsCl (2.5-3.5 eq). The mixture was stirred at 25-40° C. until no further conversion was observed. Then it was quenched with 2 M aq. HCl solution and the layers were separated. The organic layer was washed with sat. aq. NaHCO₃ solution, dried over Na₂SO₄, filtered and the filtrate was concentrated under reduced pressure. The crude product was purified via flash chromatography using heptane and EtOAc as eluents.

General Procedure 43: Double Alkylation with Bis-Tosylates

To a solution of the appropriate catechol derivative (1 eq) in DMF (15 mL/mmol) was added Cs₂CO₃ (2-3 eq) and the appropriate bis-tosylate (1-1.5 eq). The mixture was heated under N₂ at 80° C. until no further conversion was observed and then cooled to rt. The mixture was filtered, and the filtrate was purified via prep RP-HPLC using 25 mM aq. NH₄HCO₃ solution and MeCN and/or IPA as eluents.

### PREPARATIONS

### Preparation 1a (1,3-dioxolane-2,2-diyl)di(ethane-2,1-diyl) dimethanesulfonate

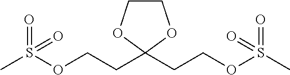

To a solution of 2-[2-(2-hydroxyethyl)-1,3-dioxolan-2-yl]ethan-1-ol (13.5 g, 83.2 mmol) in DCM (500 mL) was added TEA (35.4 mL, 25.6 g, 254 mmol) and cooled to −40° C. A solution of MsCl (16.1 mL, 23.8 g, 208.1 mmol) in DCM (500 mL) was added dropwise and stirring continued at −40° C. for 30 min. The reaction was warmed to 0° C. and quenched by the addition of sat. aq. NaHCO₃ solution. The organics were separated and the aq. phase was extracted with another portion of DCM. The combined organic extracts were washed with water, brine, dried (MgSO₄), filtered and the filtrate was concentrated in vacuo to give Preparation 1a as a white crystalline solid (25.5 g, 80.1 mmol, 96%). ¹H NMR (400 MHz, CDCl₃) δ ppm: 4.36 (t, J=6.8 Hz, 4H), 4.00 (s, 4H), 3.05 (s, 6H), 2.17 (t, J=6.8 Hz, 4H).

### Preparation 1b 2,2-bis(2-bromoethyl)-1,3-dioxolane

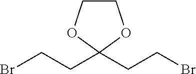 [SMILES: BrCCC1(CCBr)OCCO1]

Using General procedure 3 and 2-[2-(2-hydroxyethyl)-1,3-dioxolan-2-yl]ethanol as the appropriate alcohol, Preparation 1b was obtained. ¹H NMR (400 MHz, DMSO-d₆) δ ppm: 3.9 (s, 4H), 3.44 (m, 4H), 2.19 (m, 4H). LRMS calculated for C₇H₁₂Br₂O₂: 285.92; found 207.0 (M−HBr).

### Preparation 2a1 and Preparation 2a2

### Preparation 2aA 5-[(E)-2-(2-bromo-5-methyl-anilino)vinyl]-2,2-dimethyl-1,3-dioxane-4,6-dione

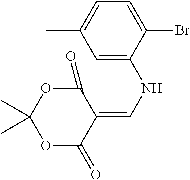 [SMILES: Cc1ccc(Br)c(NC=C2C(=O)OC(C)(C)OC2=O)c1]

To the solution of 2-bromo-5-methyl-aniline (24.4 g, 131 mmol) in EtOH (610 mL), 5-(methoxymethylene)-2,2-dimethyl-1,3-dioxane-4,6-dione (26.9 g, 144.0 mmol) was added at rt and the mixture was stirred at rt for 45 min. Then it was concentrated under reduced pressure. The residue was digerated with DIPE. The precipitate was filtered and washed with DIPE. The precipitate was dried under reduced pressure at 40° C. to give Preparation 2aA. ¹H NMR (400 MHz, DMSO-d₆) δ ppm: 11.51 (d, 1H), 8.76 (d, 1H), 7.76 (s, 1H), 7.61 (d, 1H), 7.04 (d, 1H), 2.33 (s, 3H), 1.69 (s, 6H).

### Preparation 2aB 8-bromo-5-methyl-quinolin-4-ol

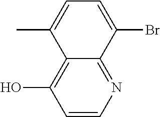

The solution of Preparation 2aA (78.5 g, 231.0 mmol) in Ph₂O (393 mL) in a 2 L 3-necked flask equipped with N₂ inlet, overhead stirrer and air cooled reflux condenser was put in a pre-heated bath, and it was stirred at 270° C. for 40 min. During the reaction slow N₂ stream was applied. The reaction mixture was allowed to cool to 100° C., and it was poured into 1.6 L well stirred heptane. The precipitate was filtered off, and taken up in the mixture of DIPE (320 mL) and heptane (160 mL). It was refluxed for 15 min, then it was allowed to cool to rt. The mixture was filtered and the precipitate was washed with DIPE. This reflux-crystallisation process was repeated. The solids were dried under reduced pressure to give Preparation 2aB. ¹H NMR (400 MHz, DMSO-d₆) δ ppm: 10.77 (br s, 1H), 7.80 (d, 1H), 7.73 (dd, 1H), 6.96 (d, 1H), 6.04 (d, 1H), 2.75 (s, 3H).

### Preparation 2a1 (5R)-5-methyl-5,6,7,8-tetrahydroquinolin-4-ol

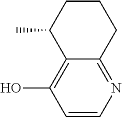

and

### Preparation 2a2 (5S)-5-methyl-5,6,7,8-tetrahydroquinolin-4-ol

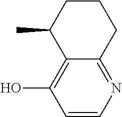

Preparation 2aB (120 g, 504 mmol) was dissolved in AcOH (1100 mL) and MeOH (500 mL). CH₃COONa×3H₂O (103 g, 756 mmol) and 10% Pd/C (12.0 g, 0.1 g/g quinolin-4-ol) was added to the mixture. The autoclave was evacuated and backfilled with N₂ (×3), then evacuated and filled with H₂. The reaction mixture was stirred under 10 bar H₂ at 50° C. for 1.5 h. The flask was evacuated and backfilled with N₂ and PtO₂ (12.0 g, 0.1 g/g quinolin-4-ol) was added in TFA (116 mL). The flask was evacuated and filled with H₂. The reaction mixture was stirred under 10 bar H₂ at 50° C. for 4 h. The reaction mixture was filtered through a pad of silica gel and washed with MeOH. The filtrate was concentrated under reduced pressure. MeOH was added and concentrated under reduced pressure to remove traces of AcOH and TFA. 6 M NH₃ solution in MeOH (90 mL) was added and the mixture was concentrated under reduced pressure. The residue was taken up in DCM-MeOH mixture (4:1) and evaporated onto silica gel. The crude product was purified via flash chromatography using NH₃/MeOH and EtOAc as eluents. The resulting intermediate was taken up in MeOH (240 mL) and iPrOH (640 mL) and stirred at 60° C. for 20 min, then heptane (250 mL) was added. The precipitate was filtered and washed with iPrOH (50 mL). The filtrate was allowed to cool to rt and the precipitate was filtered. The filtrate was concentrated under reduced pressure. The residue was taken up in DIPE (250 mL), stirred at 45° C. for 20 min, then heptane was added (250 mL) and the precipitate was filtered and dried to give a racemate. The enantiomers were separated by chiral chromatography. Column: AS-V, 100×500 mm, 20 μm, Eluents: 3:15:82 MeOH/iPrOH/heptane+0.05% DEA. The enantiomer eluting earlier was collected as Preparation 2a2. ¹H NMR (500 MHz, DMSO-d₆) δ ppm: 11.05 (br s, 1H), 7.43 (d, 1H), 5.90 (d, 1H), 2.83 (m, 1H), 2.54-2.42 (m, 2H), 1.79-1.63 (m, 2H), 1.64-1.49 (m, 2H), 1.04 (d, 3H). HRMS calculated for C₁₀H₁₃NO: 163.0997; found 164.1071 (M+H).

The enantiomer eluting later was collected and purified via flash chromatography using MeOH and EtOAc as eluents to give Preparation 2a1. HRMS calculated for C₁₀H₁₃NO: 163.0997; found 163.09939 (M+).

### Preparation 3a

### Preparation 3aA 2-(4-methoxyphenyl)-5-methyl-1,3-dioxane

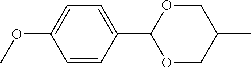

1-(dimethoxymethyl)-4-methoxy-benzene (10.0 g, 55.0 mmol) was dissolved in dry DCM (330 mL). 2-methylpropane-1,3-diol (4.07 mL, 46.2 mmol) and PPTS (1.38 g, 5.5 mmol) were added and the mixture was stirred at rt for 3 h. Then NaHCO₃ (924 mg, 11.0 mmol) was added and it was stirred at rt for 30 min. Then it was concentrated under reduced pressure. The crude product was purified via flash chromatography using heptane and EtOAc as eluents to obtain Preparation 3aA. ¹H NMR (400 MHz, DMSO-d₆) δ ppm: 7.35-7.31 (m, 2H), 6.93-6.88 (m, 2H), 5.43/5.37 (s, 1H), 4.11-4.02 (m, 2H), 3.80/3.46 (dm/t, 2H), 3.75 (s, 3H), 3.48-3.43 (m, 2H), 2.09-1.99/1.68-1.62 (m, 1H), 1.23/0.70 (d, 3H).

### Preparation 3aB (2R)-3-[(4-methoxyphenyl)methoxy]-2-methyl-propan-1-ol

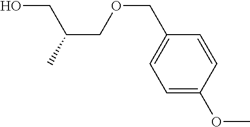

and

### Preparation 3aC (2S)-3-[(4-methoxyphenyl)methoxy]-2-methyl-propan-1-ol

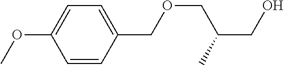

Preparation 3aA (78.0 g, 374 mmol) was dissolved in DCM (750 mL) and cooled to 0° C. 1 M DIBAL-H solution in DCM (800 mL) was added dropwise at 0° C., then it was allowed to warm to rt and stirred for 1 h. Then it was cooled to 0° C., MeOH (200 mL) was added dropwise at 0° C., then water (200 mL) was added. The mixture was stirred at rt for 1 h, then it was diluted with water (600 mL). The layers were separated. The organic layer was dried over Na₂SO₄, filtered and the filtrate was concentrated under reduced pressure. The crude product was purified via distillation (bp: 190° C., 0.45 mbar) to give a racemate. The enantiomers were separated by chiral chromatography. Column: AS-V, 10×500 mm, 20 μm, Eluents: 10:90 EtOH/heptane. The enantiomer eluting earlier was collected as Preparation 3aB. ¹H NMR (500 MHz, DMSO-d₆) δ ppm: 7.23 (m, 2H), 6.9 (m, 2H), 4.4 (t, 1H), 4.36 (s, 2H), 3.74 (s, 3H), 3.35/3.2 (dd+dd, 2H), 3.34/3.26 (t+t, 2H), 1.78 (m, 1H), 0.84 (d, 3H). HRMS calculated for Cl₂H₁₈O₃: 210.1256; found 210.12478 (M+).

The enantiomer eluting later was collected as Preparation 3aC. HRMS calculated for Cl₂H₁₈O₃: 210.1256; found 210.12489 (M+).

### Preparation 3aD tert-butyl-[(2R)-3-[(4-methoxyphenyl)methoxy]-2-methyl-propoxy]-diphenyl-silane

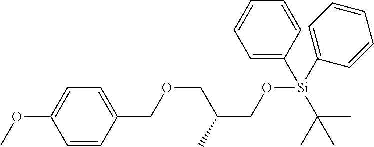 [SMILES: COc1ccc(COC[C@@H](C)CO[Si](c2ccccc2)(c2ccccc2)C(C)(C)C)cc1]

Preparation 3aC (420 mg, 2.0 mmol) was dissolved in dry THE (6 mL). Imidazole (143 mg, 2.1 mmol) and TBDPS-Cl (537 μL, 2.1 mmol) was added to the mixture and stirred at rt overnight. The reaction mixture was diluted with sat. aq. NaHCO₃ solution and extracted with EtOAc. The combined organic layers were washed with brine, dried over Na₂SO₄, filtered and the filtrate was concentrated under reduced pressure. The crude product was purified via flash chromatography using heptane and EtOAc as eluents to obtain Preparation 3aD. ¹H NMR (500 MHz, DMSO-d₆) δ ppm: 7.62-7.36 (m, 10H), 7.19 (m, 2H), 6.87 (m, 2H), 4.35/4.34 (d+d, 2H), 3.72 (s, 3H), 3.59/3.56 (dd+dd, 2H), 3.41/3.31 (dd+dd, 2H), 1.92 (m, 1H), 0.97 (s, 9H), 0.89 (d, 3H). HRMS calculated for C₂₈H₃₆O₃Si: 448.2434; found 471.23248 (M+Na).

### Preparation 3aE (2R)-3-[tert-butyl(diphenyl)silyl]oxy-2-methyl-propan-1-ol

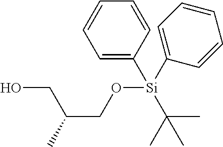 [SMILES: C[C@H](CO)CO[Si](c1ccccc1)(c1ccccc1)C(C)(C)C]

Using General procedure 28a and Preparation 3aD as the appropriate PMB derivative, Preparation 3aE was obtained. ¹H NMR (500 MHz, DMSO-d₆) δ ppm: 7.61 (m, 4H), 7.45 (dd, 2H), 7.43 (m, 4H), 4.42 (t, 1H), 3.62/3.5 (dd+dd, 2H), 3.41/3.31 (dd+dd, 2H), 1.77 (m, 1H), 0.99 (s, 9H), 0.87 (d, 3H). HRMS calculated for C₂₀H₂₈O₂Si: 328.1859; found 329.1927 (M+H).

### Preparation 3a [(2S)-3-bromo-2-methyl-propoxy]-tert-butyl-diphenyl-silane

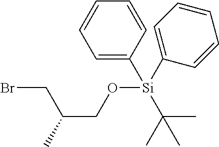 [SMILES: C[C@H](CBr)CO[Si](c1ccccc1)(c1ccccc1)C(C)(C)C]

Using General procedure 3 and Preparation 3aE as the appropriate alcohol, Preparation 3a was obtained. ¹H NMR (500 MHz, DMSO-d₆) δ ppm: 7.62 (m, 4H), 7.47 (dd, 2H), 7.45 (m, 4H), 3.62 (m, 2H), 3.57 (m, 2H), 2.03 (m, 1H), 1 (s, 9H), 0.95 (d, 3H). HRMS calculated for C₂₀H₂₇BrOSi: 390.1015; found 333.03048 (M-tBu).

### Preparation 3b bromo-[(2S)-3-[tert-butyl(diphenyl)silyl]oxy-2-methyl-propyl]zinc

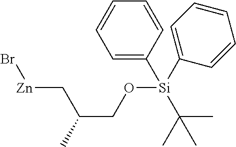

Using General procedure 4 and Preparation 3a as the appropriate bromo compound, Preparation 3b was obtained.

### Preparation 3c 1-[[(2S)-3-bromo-2-methyl-propoxy]methyl]-4-methoxy-benzene

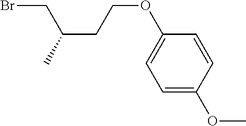

Using General procedure 3 and Preparation 3aB as the appropriate alcohol, Preparation 3c was obtained. ¹H NMR (500 MHz, DMSO-d₆) δ ppm: 7.25 (m, 2H), 6.9 (m, 2H), 4.39 (s, 2H), 3.74 (s, 3H), 3.54 (m, 2H), 3.31 (d, 2H), 2.05 (m, 1H), 0.94 (d, 3H). HRMS calculated for Cl₂H₁₇BrO₂: 272.0412; found 272.04064 (M+).

### Preparation 3d bromo-[(2S)-3-[(4-methoxyphenyl)methoxy]-2-methyl-propyl]zinc

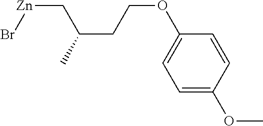

Using General procedure 4 and Preparation 3c as the appropriate bromo compound, Preparation 3d was obtained.

### Preparation 3e

### Preparation 3eA 2-[(2R)-3-[(4-methoxyphenyl)methoxy]-2-methyl-propyl]isoindoline-1,3-dione

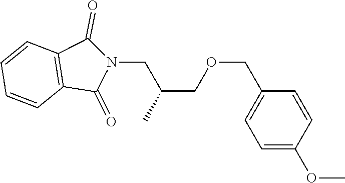 [SMILES: COc1ccc(COC[C@H](C)CN2C(=O)c3ccccc3C2=O)cc1]

Preparation 3c (54.6 g, 200 mmol) was dissolved in DMF (220 mL). Potassium phthalimide (42.6 g, 230 mmol) was added to the mixture and stirred at 50° C. for 3 h. The mixture was allowed to cool to rt. EtOAc was added to the mixture and stirred at rt for 10 min, then it was washed with water. The organic layer was dried over Na₂SO₄, filtered and the filtrate was concentrated under reduced pressure. The crude product was purified via flash chromatography using heptane and EtOAc as eluents to obtain Preparation 3eA. ¹H NMR (500 MHz, DMSO-d₆) δ ppm: 7.88-7.78 (m, 4H), 7.14 (m, 2H), 6.81 (m, 2H), 4.31/4.28 (d+d, 2H), 3.71 (s, 3H), 3.61/3.44 (dd+dd, 2H), 3.31/3.29 (dd+dd, 2H), 2.18 (m, 1H), 0.86 (d, 3H).

### Preparation 3e (2R)-3-[(4-methoxyphenyl)methoxy]-2-methyl-propan-1-amine

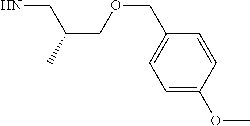

Preparation 3eA (688 mg, 2.03 mmol) was dissolved in EtOH (8 mL). Hydrazine hydrate (105 μL, 2.13 mmol) was added to the mixture and stirred under N₂ atmosphere at 75° C. for 3 h. The mixture was allowed to cool to rt and washed with sat. aq. NH₄Cl solution. The organic layer was dried over MgSO₄, filtered and the filtrate was concentrated under reduced pressure. The crude product was purified via flash chromatography using heptane and EtOAc as eluents to obtain Preparation 3e. ¹H NMR (500 MHz, DMSO-d₆) δ ppm: 7.23 (dm, 2H), 6.90 (dm, 2H), 4.35 (s, 2H), 3.74 (s, 3H), 3.32/3.20 (dd+dd, 2H), 2.52/2.37 (dd+dd, 2H), 1.65 (m, 1H), 1.32 (m, 2H), 0.83 (d, 3H). HRMS calculated for Cl₂H₁₉NO₂: 209.1416; found 210.1492 (M+H).

### Preparation 4a

### Preparation 4aA 2″-bromodispiro[[1,3]dioxolane-2,1′-cyclohexane-4′,1″-indene]

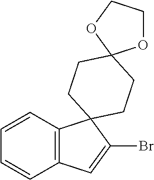 [SMILES: BrC1=Cc2ccccc2C12CCC1(CC2)OCCO1]

Using General procedure 8a and 2-bromo-1H-indene as the appropriate indene, Preparation 4aA was obtained. ¹H NMR (500 MHz, DMSO-d₆) δ ppm: 7.68 (dm, 1H), 7.36 (dm, 1H), 7.28 (m, 1H), 7.20 (m, 1H), 7.04 (s, 1H), 3.99-3.92 (m, 4H), 2.11/1.17 (m+m, 4H), 2.11/1.87 (m+m, 4H). HRMS calculated for C16H17 Br O2: 320.0412; found 321.0484 (M+H).

### Preparation 4a 2′-bromospiro[cyclohexane-1,1′-inden]-4-one

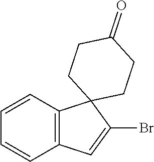 [SMILES: O=C1CCC2(CC1)C(Br)=Cc1ccccc12]

Using General procedure 9 and Preparation 4aA as the appropriate ketal, Preparation 4a was obtained. ¹H NMR (400 MHz, DMSO-d₆) δ ppm: 7.91 (dm, 1H), 7.40 (dm, 1H), 7.32 (m, 1H), 7.22 (m, 1H), 7.11 (s, 1H), 2.93/2.49 (m+m, 4H), 2.22/1.58 (m+m, 4H). LRMS calculated for C₁₄H₁₃BrO: 276.02; found 276.1 (M+).

### Preparation 5a

### Preparation 5aA (1′s,1″s)-2″-bromodispiro[imidazolidine-4,1′-cyclohexane-4′,1″-indene]-2,5-dione

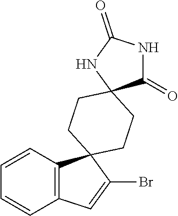 [SMILES: O=C1NC(=O)[C@]2(CC[C@@]3(CC2)C(Br)=Cc2ccccc23)N1]

Using General procedure 14 and Preparation 4a as the appropriate ketone, a 4:1 mixture of diastereoisomers was obtained. DME (7.5 mL/mmol) was added and the mixture was stirred at 82° C. for 2 h, then slowly cooled to 20° C. and stirred for 2 h. Then it was filtered and the filtrate was concentrated under reduced pressure. Heptane (1.4 mL/mmol) was added and it was stirred at rt for 1 h. The precipitate was filtered, washed with heptane to obtain Preparation 5aA as a single diastereoisomer. ¹H NMR (500 MHz, DMSO-d₆) δ ppm: 10.80 (br s, 1H), 8.96 (s, 1H), 7.72 (dm, 1H), 7.38 (m, 1H), 7.25 (m, 1H), 7.06 (m, 2H), 2.38/1.76 (m+m, 4H), 2.12/1.17 (m+m, 4H). HRMS calculated for C₁₆H₁₅BrN₂O₂: 346.0317; found 347.0392 (M+H).

### Preparation 5a (1s,4s)-4-amino-2′-bromospiro[cyclohexane-1,1′-indene]-4-carboxylic acid

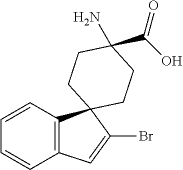 [SMILES: N[C@]1(C(=O)O)CC[C@@]2(CC1)C(Br)=Cc1ccccc12]

Using General procedure 15 and Preparation 5aA as the appropriate hydantoin, Preparation 5a was obtained as a single diastereoisomer. ¹H NMR (400 MHz, DMSO-d₆) δ ppm: 8.05 (d, J=7.60 Hz, 1H), 7.81 (br s, 2H), 7.37 (dd, J=7.4, 1.3 Hz, 1H), 7.30 (td, J=7.4, 1.0 Hz, 1H), 7.22 (td, J=7.5, 1.4 Hz, 1H), 7.05 (s, 1H), 2.65 (td, J=14.7, 5.0 Hz, 2H), 2.06 (td, J=14.1, 4.4 Hz, 2H), 1.82-1.72 (m, 2H), 1.11-1.02 (m, 2H). HRMS calculated for C₁₅H₁₆NO₂Br: 321.0364; found 322.0435 (M+H).

### Preparation 5b

### Preparation 5bA 6-bromo-6,7-dihydro-2H,5H-indeno[5,6-d][1,3]dioxol-5-one

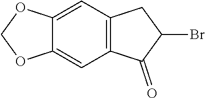 [SMILES: O=C1c2cc3c(cc2CC1Br)OCO3]

Using General procedure 5 and 5,6-dihydrocyclopenta[f][1,3]benzodioxol-7-one as the appropriate indan-1-one, Preparation 5bA was obtained. ¹H NMR (500 MHz, DMSO-d₆) δ ppm: 7.13 (s, 1H), 7.08 (d, 1H), 6.21/6.20 (d+d, 2H), 4.97 (dd, 1H), 3.76/3.19 (dd+dd, 2H). HRMS calculated for C₁₀H₇BrO₃: 253.9579; found 254.9645 (M+H).

### Preparation 5bB 6-bromo-6,7-dihydro-2H,5H-indeno[5,6-d][1,3]dioxol-5-ol

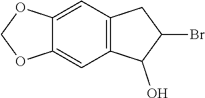 [SMILES: OC1c2cc3c(cc2CC1Br)OCO3]

Preparation 5bA (69.0 g, 271 mmol) was dissolved in MeOH (740 mL) and cooled with ice-bath (0-5° C.). NaBH₄ (10.2 g, 271 mmol) was added to the mixture portionwise, then the mixture was stirred at 0° C. for 30 min. The reaction mixture was diluted with water (800 mL). The precipitate was filtered, washed with water and dried to give Preparation 5bB. ¹H NMR (500 MHz, DMSO-d₆) δ ppm: 6.82 (s, 1H), 6.81 (s, 1H), 5.98 (d, 2H), 4.82 (dd, 1H), 4.79 (d, 1H), 3.28/3.08 (dd+dd, 2H). HRMS calculated for C₁₀H₉BrO₃: 255.9735; found 255.97248 (M+).

### Preparation 5bC 6-bromo-2H,5H-indeno[5,6-d][1,3]dioxole

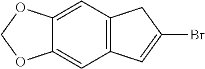 [SMILES: BrC1=Cc2cc3c(cc2C1)OCO3]

Using General procedure 7 and Preparation 5bB as the appropriate indane and dry CHCl₃ instead of toluene, Preparation 5bC was obtained. ¹H NMR (500 MHz, DMSO-d₆) δ ppm: 7.03 (t, 1H), 6.97 (t, 1H), 6.95 (s, 1H), 5.99 (s, 2H), 3.58 (d, 2H). HRMS calculated for C₁₀H₇BrO₂: 237.9629; found 237.95976 (M+).

### Preparation 5bD 6″-bromo-2″H-dispiro[[1,3]dioxolane-2,1′-cyclohexane-4′,5″-indeno[5,6-d][1,3]dioxole]

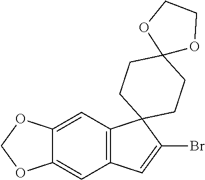 [SMILES: BrC1=Cc2cc3c(cc2C12CCC1(CC2)OCCO1)OCO3]

Using General procedure 8a and Preparation 5bC as the appropriate indene, Preparation 5bD was obtained. ¹H NMR (500 MHz, DMSO-d₆) δ ppm: 7.22 (s, 1H), 6.96 (s, 1H), 6.90 (s, 1H), 6.01 (s, 2H), 3.98-3.91 (m, 4H), 2.06/1.18 (m+m, 4H), 2.04/1.86 (m+m, 4H). HRMS calculated for C₁₇H₁₇BrO₄: 364.031; found 365.0383 (M+H).

### Preparation 5bE 6′-bromo-2′H-spiro[cyclohexane-1,5′-indeno[5,6-d][1,3]dioxol]-4-one

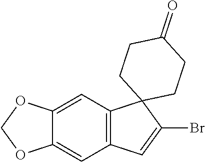 [SMILES: O=C1CCC2(CC1)C(Br)=Cc1cc3c(cc12)OCO3]

Using General procedure 9 and Preparation 5bD as the appropriate ketal, Preparation 5bE was obtained. ¹H NMR (500 MHz, DMSO-d₆) δ ppm: 7.63 (s, 1H), 6.99 (s, 1H), 6.96 (s, 1H), 6.02 (s, 2H), 2.90/2.47 (m+m, 4H), 2.15/1.59 (m+m, 4H). HRMS calculated for C₁₅H₁₃BrO₃: 320.0048; found 320.0024 (M+).

### Preparation 5bF 6″-bromo-2″H-dispiro[imidazolidine-4,1′-cyclohexane-4′,5″-indeno[5,6-d][1,3]dioxole]-2,5-dione

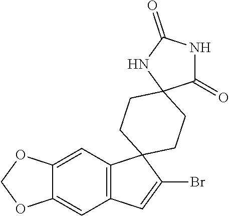 [SMILES: O=C1NC(=O)C2(CCC3(CC2)C(Br)=Cc2cc4c(cc23)OCO4)N1]

Using General procedure 14 and Preparation 5bE as the appropriate ketone, Preparation 5bF was obtained as a 4:1 mixture of diastereoisomers. ¹H NMR (500 MHz, DMSO-d₆) δ ppm: 10.81 (s, 1H), 8.95 (s, 1H), 7.23 (s, 1H), 6.99 (s, 1H), 6.92 (s, 1H), 6.03 (s, 2H), 2.3/1.75 (td+d, 4H), 2.07/1.16 (td+d, 4H). HRMS calculated for C₁₇H₁₅BrN₂O₄: 390.0215; found 391.0286 and 391.0258 (M+H).

### Preparation 5b 4-amino-6′-bromo-2′H-spiro[cyclohexane-1,5′-indeno[5,6-d][1,3]dioxole]-4-carboxylic acid

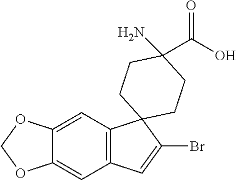 [SMILES: NC1(C(=O)O)CCC2(CC1)C(Br)=Cc1cc3c(cc12)OCO3]

Using General procedure 15 and Preparation 5bF as the appropriate hydantoin, Preparation 5b was obtained as a 4:1 mixture of diastereoisomers. ¹H NMR (500 MHz, DMSO-d₆) δ ppm: 7.70/7.69 (s/s, 1H), 6.96/6.92 (s/s, 1H), 6.89/6.85 (s/s, 1H), 6.01/6.00 (s/s, 2H), 2.63-0.97 (m, 8H). HRMS calculated for C₁₆H₁₆BrNO₄: 365.0263; found 366.0644 and 366.0337 (M+H).

### Preparation 6a and Preparation 6b and Preparation 6c

### Preparation 6aA (1s,4s)-2′-bromo-4-(3-chloroanilino)spiro[cyclohexane-1,1′-indene]-4-carbonitrile

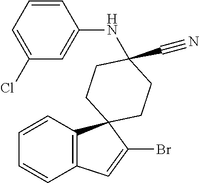 [SMILES: N#C[C@]1(Nc2cccc(Cl)c2)CC[C@]2(CC1)C(Br)=Cc1ccccc12]

and

### Preparation 6bA (1r,4r)-2′-bromo-4-(3-chloroanilino)spiro[cyclohexane-1,1′-indene]-4-carbonitrile

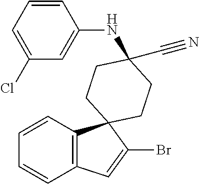 [SMILES: N#C[C@]1(Nc2cccc(Cl)c2)CC[C@@]2(CC1)C(Br)=Cc1ccccc12]

Using General procedure 11 and Preparation 4a as the appropriate ketone and 3-chloroaniline as the appropriate aniline, a mixture of diastereoisomers was obtained. The diastereoisomers were separated via flash chromatography using heptane and EtOAc as eluents. The diastereoisomer eluting earlier was collected as Preparation 6aA. ¹H NMR (500 MHz, DMSO-d₆) δ ppm: 7.82-7.17 (m, 4H), 7.24 (t, 1H), 7.12/7.06 (s, 1H), 6.96/6.94 (t, 1H), 6.92/6.90 (dm, 1H), 6.79 (dm, 1H), 6.59/6.57 (s, 1H), 2.64-1.19 (m, 8H). HRMS calculated for C₂₁H₁₈BrClN₂: 412.0342; found 413.0415 (M+H).

The diastereoisomer eluting later was collected as Preparation 6bA. ¹H NMR (500 MHz, DMSO-d₆) δ ppm: 7.82-7.17 (m, 4H), 7.24 (t, 1H), 7.12/7.06 (s, 1H), 6.96/6.94 (t, 1H), 6.92/6.90 (dm, 1H), 6.79 (dm, 1H), 6.59/6.57 (s, 1H), 2.64-1.19 (m, 8H). HRMS calculated for C₂₁H₁₈BrClN₂: 412.0342; found 413.0401 (M+H).

### Preparation 6aB (1s,4s)-2′-bromo-4-(3-chloroanilino)spiro[cyclohexane-1,1′-indene]-4-carboxamide

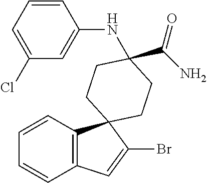 [SMILES: NC(=O)[C@]1(Nc2cccc(Cl)c2)CC[C@]2(CC1)C(Br)=Cc1ccccc12]

Using General procedure 12b and Preparation 6aA as the appropriate nitrile, Preparation 6aB was obtained. ¹H NMR (500 MHz, DMSO-d₆) δ ppm: 7.76 (d, 1H), 7.36 (dd, 1H), 7.33/7.25 (br+br, 2H), 7.29 (td, 1H), 7.23 (td, 1H), 7.12 (t, 1H), 7.02 (s, 1H), 6.70 (t, 1H), 6.62 (dm, 1H), 6.60 (dm, 1H), 2.46/2.10 (td+d, 4H), 2.13/0.94 (t+d, 4H). HRMS calculated for C₂₁H₂₀BrClN₂O: 430.0447; found 431.0517 (M+H).

### Preparation 6a (1s,4s)-2′-bromo-4-(3-chloroanilino)spiro[cyclohexane-1,1′-indene]-4-carboxylic acid

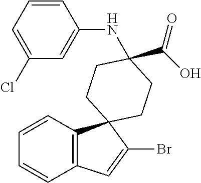 [SMILES: O=C(O)[C@]1(Nc2cccc(Cl)c2)CC[C@]2(CC1)C(Br)=Cc1ccccc12]

Using General procedure 13 and Preparation 6aB as the appropriate amide, Preparation 6a was obtained. ¹H NMR (500 MHz, DMSO-d₆) δ ppm: 7.77 (d, 1H), 7.35 (dd, 1H), 7.28 (t, 1H), 7.21 (td, 1H), 7.01 (t, 1H), 7.01 (s, 1H), 6.62 (t, 1H), 6.56 (dd, 1H), 6.46 (dd, 1H), 2.40/2.17 (t+d, 4H), 2.16/0.92 (t+d, 4H). HRMS calculated for C₂₁H₁₉BrClNO₂: 431.0288; found 432.0358 (M+H).

### Preparation 6bB (1r,4r)-2′-bromo-4-(3-chloroanilino)spiro[cyclohexane-1,1′-indene]-4-carboxamide

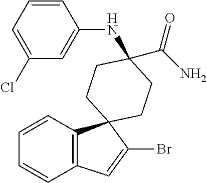 [SMILES: NC(=O)[C@]1(Nc2cccc(Cl)c2)CC[C@@]2(CC1)C(Br)=Cc1ccccc12]

Using General procedure 12b and Preparation 6bA as the appropriate nitrile, Preparation 6bB was obtained. ¹H NMR (500 MHz, DMSO-d₆) δ ppm: 7.77 (d, 1H), 7.49/7.16 (br+br, 2H), 7.33 (dd, 1H), 7.26 (td, 1H), 7.20 (td, 1H), 7.09 (t, 1H), 7.02 (s, 1H), 6.74 (t, 1H), 6.65 (dm, 1H), 6.60 (dm, 1H), 6.23 (s, 1H), 2.56/2.02 (m+m, 4H), 2.08/1.36 (m+m, 4H). HRMS calculated for C₂₁H₂₀BrClN₂O: 430.0447; found 431.0526 (M+H).

### Preparation 6b (1r,4r)-2′-bromo-4-(3-chloroanilino)spiro[cyclohexane-1,1′-indene]-4-carboxylic acid

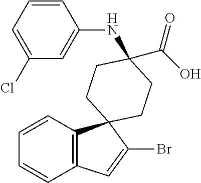 [SMILES: O=C(O)[C@]1(Nc2cccc(Cl)c2)CC[C@@]2(CC1)C(Br)=Cc1ccccc12]

Using General procedure 13 and Preparation 6bB as the appropriate amide, Preparation 6b was obtained. ¹H NMR (500 MHz, DMSO-d₆) δ ppm: 7.70 (d, 1H), 7.34 (dd, 1H), 7.27 (td, 1H), 7.21 (td, 1H), 7.09 (t, 1H), 7.03 (s, 1H), 6.75 (t, 1H), 6.66 (dd, 1H), 6.54 (dd, 1H), 6.39 (br, 1H), 2.62/2.06 (m+m, 4H), 2.04/1.32 (m+m, 4H). HRMS calculated for C₂₁H₁₉BrClNO₂: 431.0288; found 432.0363 (M+H).

### Preparation 6c methyl (1s,4s)-2′-bromo-4-(3-chloroanilino)spiro[cyclohexane-1,1′-indene]-4-carboxylate

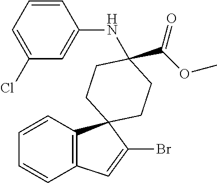 [SMILES: COC(=O)[C@]1(Nc2cccc(Cl)c2)CC[C@]2(CC1)C(Br)=Cc1ccccc12]

Using General procedure 17a and Preparation 6a as the appropriate amino acid, Preparation 6c was obtained. ¹H NMR (500 MHz, DMSO-d₆) δ ppm: 7.71 (d, 1H), 7.37 (dd, 1H), 7.30 (t, 1H), 7.23 (td, 1H), 7.10 (t, 1H), 7.04 (s, 1H), 6.61 (t, 1H), 6.60 (dm, 1H), 6.48 (dm, 1H), 3.69 (s, 3H), 2.40/2.27 (td+br d, 4H), 2.21/0.99 (td+br d, 4H). HRMS calculated for C₂₂H₂₁BrClNO₂: 445.0444; found 446.0506 (M+H).

### Preparation 7a (1s,4s)-4-(3-chloroanilino)-4-(methoxycarbonyl)spiro[cyclohexane-1,1′-indene]-2′-carboxylic acid

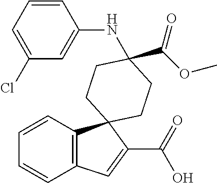 [SMILES: COC(=O)[C@]1(Nc2cccc(Cl)c2)CC[C@]2(CC1)C(C(=O)O)=Cc1ccccc12]

Preparation 6c (230 mg, 0.55 mmol) was dissolved in DMF (6 mL) and water (2 mL).

Pd(OAc)₂ (11.2 mg, 0.05 mmol), dppp (24.8 mg, 0.06 mmol) and TEA (210 μL, 1.5 mmol) were added. The mixture was placed into an autoclave. The autoclave was evacuated and filled with 10 bar CO. The mixture was stirred at 80° C. overnight. The reaction mixture was concentrated under reduced pressure. The crude product was purified via flash chromatography using heptane and EtOAc as eluents to obtain Preparation 7a. ¹H NMR (500 MHz, DMSO-d₆) δ ppm: 12.42 (br s, 1H), 7.79 (m, 1H), 7.64 (s, 1H), 7.59 (m, 1H), 7.37 (m, 2H), 7.08 (t, 1H), 6.62 (t, 1H), 6.58 (dd, 1H), 6.45 (dd, 1H), 6.32 (br s, 1H), 3.69 (s, 3H), 2.79/0.94 (td+d, 4H), 2.37/2.23 (td+d, 4H). HRMS calculated for C₂₃H₂₂ClNO₄: 411.1237; found 412.1312 (M+H).

### Preparation 7b methyl (1s,4s)-4-(3-chloroanilino)-2′-(hydroxymethyl)spiro[cyclohexane-1,1′-indene]-4-carboxylate

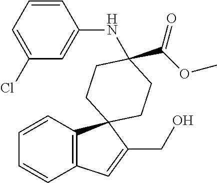 [SMILES: COC(=O)[C@]1(Nc2cccc(Cl)c2)CC[C@]2(CC1)C(CO)=Cc1ccccc12]

Preparation 7a (6.80 g, 16.5 mmol) was dissolved in THE (100 mL). BH₃×SMe₂ (3.90 mL, 41.3 mmol) was added to the mixture and stirred at rt for 80 min. The reaction was quenched by the addition of MeOH, water and AcOH. The mixture was concentrated under reduced pressure. The crude product was purified via flash chromatography using heptane and EtOAc as eluents to obtain Preparation 7b. ¹H NMR (500 MHz, DMSO-d₆) δ ppm: 7.63 (dm, 1H), 7.32 (dm, 1H), 7.23 (m, 1H), 7.12 (m, 1H), 7.09 (t, 1H), 6.63 (t, 1H), 6.61 (t, 1H), 6.60 (dm, 1H), 6.46 (dm, 1H), 6.39 (s, 1H), 5.00 (t, 1H), 4.27 (dd, 2H), 3.69 (s, 3H), 2.39/2.18 (m+m, 4H), 2.11/1.00 (m+m, 4H). HRMS calculated for C₂₃H₂₄ClNO₃: 397.1445; found 398.1532 (M+H).

### Preparation 8a

### Preparation 8aA methyl (1r,4r)-4-(3-chloroanilino)-2′-[(E)-2-ethoxyethenyl]spiro[cyclohexane-1,1′-indene]-4-carboxylate

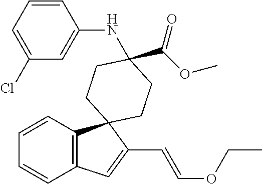 [SMILES: CCO/C=C/C1=Cc2ccccc2[C@]12CC[C@@](Nc1cccc(Cl)c1)(C(=O)OC)CC2]

To a solution of Preparation 6c (1.47 g, 3.29 mmol, 1 eq) in 1,4-dioxane (25 mL) and water (5 mL) was added 2-[(E)-2-ethoxyethenyl]-4,4,5,5-tetramethyl-1,3,2-dioxaborolane (0.91 mL, 4.28 mmol, 1.3 eq) and K₃PO₄ (2.79 g, 13.16 mmol, 4 eq). The mixture was sparged with N₂ (10 min), then Pd(dppf)Cl₂×DCM (134 mg, 0.16 mmol, 0.05 eq) was added and the mixture was heated at 80° C. for 2 h. Then the reaction was partitioned between EtOAc and brine. The organic phase was separated, and the aq. phase was extracted with another portion of EtOAc. The combined organic extracts were washed with brine, dried (MgSO₄) and concentrated in vacuo. Purification by automated flash chromatography (CombiFlash Rf, 24 g RediSep™ silica cartridge) eluting with a gradient of 0-40% EtOAc in heptane afforded Preparation 8aA as a pale yellow powder (1.25 g, 2.86 mmol, 87%). LRMS calculated for C₂₆H₂₈ClNO₃: 437; found 438 (M+H).

### Preparation 8a methyl (1r,4r)-4-(3-chloroanilino)-2′-(2-hydroxyethyl)spiro[cyclohexane-1,1′-indene]-4-carboxylate

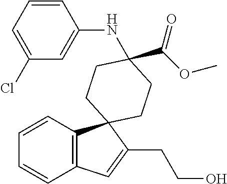 [SMILES: COC(=O)[C@]1(Nc2cccc(Cl)c2)CC[C@]2(CC1)C(CCO)=Cc1ccccc12]

To a solution of Preparation 8aA (470 mg, 1.07 mmol, 1 eq) in acetone (10 mL) was added 2 M aq. HCl solution (3 mL, 6 mmol, 5.6 eq) and the mixture was heated at 45° C. for 45 min. After cooling, the mixture was partitioned between DCM and 2 M aq. NaOH solution. The organic phase was separated and the aq. phase was extracted with further portions of DCM. The combined organic extracts were washed with brine, dried (MgSO₄) and concentrated in vacuo. The residue was dissolved in THE (10 mL), then NaBH₄ (56 mg, 1.49 mmol, 1.4 eq) was added and the mixture was stirred at rt for 2 h. After quenching with water, the mixture was partitioned between EtOAc and water. The organic phase was separated and the aq. phase was extracted with further portions of EtOAc. The combined organic extracts were washed with brine, dried (MgSO₄) and concentrated in vacuo. Purification by automated flash column chromatography (CombiFlash Rf, 12 g RediSep™ silica cartridge) eluting with a gradient of 0-60% EtOAc in heptane afforded Preparation 8a. LRMS calculated for C₂₄H₂₆ClNO₃: 411; found 412 (M+H). ¹H NMR (400 MHz, DMSO-d₆) δ ppm: 7.65 (d, J=7.5 Hz, 1H), 7.28 (dd, J=7.4, 1.3 Hz, 1H), 7.23 (td, J=7.4, 0.9 Hz, 1H), 7.14-7.08 (m, 2H), 6.65 (t, J=2.1 Hz, 1H), 6.60 (ddd, J=7.8, 2.0, 0.8 Hz, 1H), 6.51-6.45 (m, 3H), 4.71 (t, J=5.2 Hz, 1H), 3.73-3.65 (m, 5H), 2.46-2.35 (m, 4H), 2.27-2.06 (m, 4H), 0.93-0.86 (m, 2H).

### Preparation 8b

### Preparation 8bA methyl (1r,4r)-4-(3-chloroanilino)-2′-(2-hydroxyethyl)-2′,3′-dihydrospiro[cyclohexane-1,1′-indene]-4-carboxylate

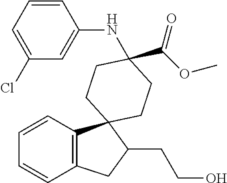

Using General Procedure 19 and Preparation 8a (150 mg, 0.36 mmol, 1 eq) as the appropriate indene and EtOH instead of EtOAc, Preparation 8bA was obtained as a racemate, isolated as a colourless gum (148 mg, 0.36 mmol, 98%). LRMS calculated for C₂₄H₂₈ClNO₃: 413; found 414 (M+H). ¹H NMR (400 MHz, DMSO-d₆) δ ppm: 7.43-7.35 (m, 1H), 7.24-7.11 (m, 3H), 7.07 (t, J=8.1 Hz, 1H), 6.62-6.55 (m, 2H), 6.48-6.43 (m, 1H), 6.34 (s, 1H), 4.49 (t, J=5.2 Hz, 1H), 3.66 (s, 3H), 3.59-3.50 (m, 1H), 3.50-3.40 (m, 1H), 2.95 (dd, J=15.7, 7.3 Hz, 1H), 2.58-2.38 (m, 2H), 2.15-1.86 (m, 5H), 1.78-1.64 (m, 2H), 1.49-1.40 (m, 1H), 1.37-1.22 (m, 2H).

### Preparation 8b methyl (1r,4r)-4-(3-chloroanilino)-2′-{2-[(methanesulfonyl)oxy]ethyl}-2′,3′-dihydrospiro[cyclohexane-1,1′-indene]-4-carboxylate

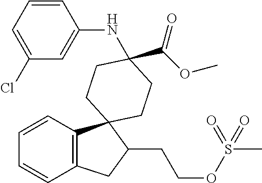

To a solution of Preparation 8bA (83 mg, 0.2 mmol, 1 eq) in DCM (3 mL) was added TEA (34 μL, 0.24 mmol, 1.2 eq) followed by the dropwise addition of MsCl (31 μL, 0.4 mmol, 2 eq) and the mixture was stirred at rt for 2 h. Then it was partitioned between DCM and water, and the organic phase was washed with with sat. aq. NaHCO₃ solution, brine, dried (PTFE phase separator) and concentrated in vacuo to afford Preparation 8b. LRMS calculated for C₂₅H₃₀ClNO₅S: 491; found 492 (M+H). ¹H NMR (400 MHz, DMSO-d₆) δ ppm: 7.46-7.39 (m, 1H), 7.25-7.14 (m, 3H), 7.08 (t, J=8.1 Hz, 1H), 6.63-6.55 (m, 2H), 6.49-6.43 (m, 1H), 6.34 (s, 1H), 4.38-4.25 (m, 2H), 3.66 (s, 3H), 3.20 (s, 3H), 3.00 (dd, J=15.7, 7.2 Hz, 1H), 2.62 (dd, J=15.7, 8.6 Hz, 1H), 2.49-2.39 (m, 1H), 2.15-1.89 (m, 6H), 1.75-1.64 (m, 1H), 1.64-1.44 (m, 2H), 1.38-1.29 (m, 1H).

### Preparation 9a

### Preparation 9aA bromido(3-{[tert-butyl(dimethyl)silyl]oxy}propyl)zinc

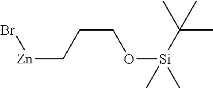

Using General procedure 4 with (3-bromopropoxy)(tert-butyl)dimethylsilane as the appropriate bromo compound, Preparation 9aA was obtained.

### Preparation 9aB methyl (1r,4r)-2′-(3-{[tert-butyl(dimethyl)silyl]oxy}propyl)-4-(3-chloroanilino)spiro[cyclohexane-1,1′-indene]-4-carboxylate

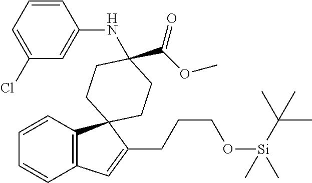 [SMILES: COC(=O)[C@]1(Nc2cccc(Cl)c2)CC[C@]2(CC1)C(CCCO[Si](C)(C)C(C)(C)C)=Cc1ccccc12]

Using General procedure 27a and Preparation 6c as the appropriate 2-bromo-indene and Preparation 9aA as the appropriate zinc reagent, Preparation 9aB was isolated as a colourless gum (152 mg, 0.28 mmol, 63%). LRMS calculated for C₃₁H₄₂ClNO₃Si: 539; found 540 (M+H).

### Preparation 9a methyl (1r,4r)-4-(3-chloroanilino)-2′-(3-hydroxypropyl)spiro[cyclohexane-1,1′-indene]-4-carboxylate

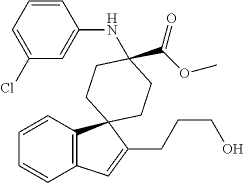 [SMILES: COC(=O)[C@]1(Nc2cccc(Cl)c2)CC[C@]2(CC1)C(CCCO)=Cc1ccccc12]

Using General procedure 29 and Preparation 9aB as the appropriate silyl derivative, Preparation 9a was isolated as a white foam (79 mg, 0.19 mmol, 63%). LRMS calculated for C₂₅H₂₈ClNO₃: 425; found 426 (M+H). ¹H NMR (400 MHz, DMSO-d₆) δ ppm: 7.65 (d, J=7.5 Hz, 1H), 7.29 (dd, J=7.4, 1.2 Hz, 1H), 7.23 (td, J=7.4, 0.9 Hz, 1H), 7.14-7.07 (m, 2H), 6.64 (t, J=2.1 Hz, 1H), 6.62-6.58 (m, 1H), 6.51-6.43 (m, 3H), 4.50 (t, J=5.1 Hz, 1H), 3.70 (s, 3H), 3.55-3.48 (m, 2H), 2.48-2.36 (m, 2H), 2.28-2.08 (m, 6H), 1.83-1.74 (m, 2H), 0.94-0.86 (m, 2H).

### Preparation 9b methyl (1r,4r)-4-(3-chloroanilino)-2′-(3-hydroxypropyl)-2′,3′-dihydrospiro[cyclohexane-1,1′-indene]-4-carboxylate

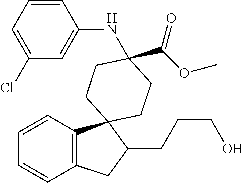

To a solution of Preparation 9a (675 mg, 1.58 mmol, 1 eq) in EtOH (30 mL) was added 5% Pt/C (300 mg, 0.08 mmol, 0.05 eq) under a N₂ atmosphere. The mixture was evacuated and backfilled with N₂ (×3), then evacuated and backfilled with H₂ and shaken for 7.5 h at rt under an atmosphere of H₂. The reaction was filtered through celite, washed with EtOH. The filtrate was concentrated under reduced pressure. Purification by automated flash chromatography (CombiFlash Rf, 12 g Gold RediSep™ silica cartridge) eluting with a gradient of 15-50% EtOAc in heptane afforded a racemate, Preparation 9b as a white foam (421 mg, 0.98 mmol, 62%). LRMS calculated for C₂₅H₃₀ClNO₃: 427; found 428 (M+H). ¹H NMR (400 MHz, DMSO-d₆) δ 7.40-7.33 (m, 1H), 7.24-7.11 (m, 3H), 7.07 (t, J=8.0 Hz, 1H), 6.62-6.55 (m, 2H), 6.49-6.44 (m, 1H), 6.32 (s, 1H), 4.40 (t, J=5.1 Hz, 1H), 3.66 (s, 3H), 3.48-3.37 (m, 2H), 2.97 (dd, J=15.7, 7.2 Hz, 1H), 2.58-2.50 (m, 1H), 2.49-2.38 (m, 1H), 2.19-2.08 (m, 1H), 2.04-1.84 (m, 4H), 1.80-1.69 (m, 1H), 1.64-1.31 (m, 5H), 1.22-1.07 (m, 1H).

### Preparation 9b1 methyl (1r,2′R,4R)-4-(3-chloroanilino)-2′-(3-hydroxypropyl)-2′,3′-dihydrospiro[cyclohexane-1,1′-indene]-4-carboxylate

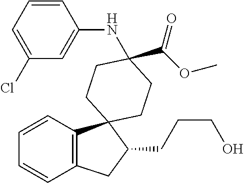 [SMILES: COC(=O)[C@]1(Nc2cccc(Cl)c2)CC[C@@]2(CC1)c1ccccc1C[C@H]2CCCO]

### Preparation 9b2 methyl (1r,2′S,4S)-4-(3-chloroanilino)-2′-(3-hydroxypropyl)-2′,3′-dihydrospiro[cyclohexane-1,1′-indene]-4-carboxylate

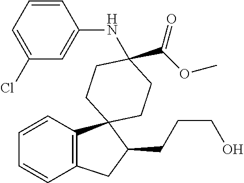 [SMILES: COC(=O)[C@]1(Nc2cccc(Cl)c2)CC[C@@]2(CC1)c1ccccc1C[C@@H]2CCCO]

The enantiomers of Preparation 9b were separated by chiral chromatography. Column: AD, 100×500 mm, 20 μm, Eluents: 10:90 EtOH/heptane. The enantiomer eluting earlier was collected as Preparation 9b1. HRMS calculated for C₂₅H₃₀ClNO₃: 427.1914; found 428.1990 (M+H).

The enantiomer eluting later was collected as Preparation 9b2. HRMS calculated for C₂₅H₃₀ClNO₃: 427.1914; found 428.1988 (M+H).

### Preparation 9c methyl (1r,4r)-4-(3-chloroanilino)-2′-{3-[(methanesulfonyl)oxy]propyl}-2′,3′-dihydrospiro[cyclohexane-1,1′-indene]-4-carboxylate

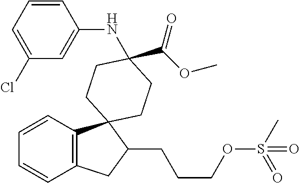

To a solution of Preparation 9b (390 mg, 0.91 mmol, 1 eq) in DCM (10 mL) was added TEA (253 μL, 1.82 mmol, 2 eq). The mixture was cooled to 0° C. before the dropwise addition of MsCl (92 μL, 1.18 mmol, 1.3 eq) and then stirred at rt for 1 h. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-50% EtOAc in heptane afforded a racemate, Preparation 9c. LRMS calculated for C₂₆H₃₂ClNO₅S: 505.17; found 506.18 (M+H). ¹H NMR (400 MHz, DMSO-d₆) δ ppm: 7.42-7.35 (m, 1H), 7.24-7.12 (m, 3H), 7.07 (t, J=8.0 Hz, 1H), 6.61-6.55 (m, 2H), 6.49-6.44 (m, 1H), 6.32 (s, 1H), 4.23 (t, J=6.4 Hz, 2H), 3.66 (s, 3H), 3.17 (s, 3H), 2.99 (dd, J=15.7, 7.2 Hz, 1H), 2.55 (dd, J=15.7, 7.8 Hz, 1H), 2.48-2.38 (m, 1H), 2.19-2.08 (m, 1H), 2.06-1.53 (m, 8H), 1.51-1.33 (m, 2H), 1.29-1.16 (m, 1H).

### Preparation 10a

### Preparation 10aA methyl (1r,4R)-4-(3-chloroanilino)-2′-{(2R)-3-[(4-methoxyphenyl)methoxy]-2-methylpropyl}spiro[cyclohexane-1,1′-indene]-4-carboxylate

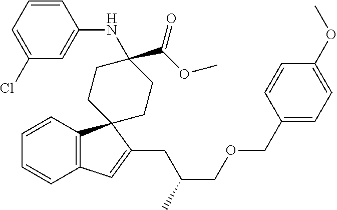 [SMILES: COc1ccc(COC[C@H](C)CC2=Cc3ccccc3[C@]23CC[C@@](Nc2cccc(Cl)c2)(C(=O)OC)CC3)cc1]

Using General procedure 27a and Preparation 6c as the appropriate 2-bromo-indene derivative and Preparation 3d as the appropriate Zn reagent, Preparation 10aA was obtained. ¹H NMR (500 MHz, DMSO-d₆) δ ppm: 7.70-7.03 (m, 4H), 7.22 (m, 2H), 7.09 (t, 1H), 6.85 (d, 2H), 6.64 (t, 1H), 6.59 (dd, 1H), 6.48 (dd, 1H), 6.43 (s, 1H), 6.43 (s, 1H), 4.40/4.37 (d, 2H), 3.72 (s, 3H), 3.69 (s, 3H), 3.33/3.30 (m, 2H), 2.47-0.78 (m, 8H), 2.35/1.99 (m, 2H), 2.17 (m, 1H), 0.94 (d, 3H). HRMS calculated for C₃₄H₃₈ClNO₄: 559.249; found 560.2557 (M+H).

### Preparation 10a methyl (1r,4R)-4-(3-chloroanilino)-2′-[(2R)-3-hydroxy-2-methylpropyl]spiro[cyclohexane-1,1′-indene]-4-carboxylate

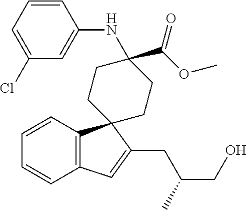 [SMILES: COC(=O)[C@]1(Nc2cccc(Cl)c2)CC[C@]2(CC1)C(C[C@@H](C)CO)=Cc1ccccc12]

Using General procedure 28b and Preparation 10aA as the appropriate PMB derivative, Preparation 10a was obtained. ¹H NMR (500 MHz, DMSO-d₆) δ ppm: 7.69-7.07 (m, 4H), 7.09 (t, 1H), 6.63 (t, 1H), 6.59 (dm, 1H), 6.47 (dm, 1H), 6.46 (s, 1H), 6.43 (s, 1H), 4.54 (t, 1H), 3.69 (s, 3H), 3.35/3.30 (m+m, 2H), 2.48-0.81 (m, 8H), 2.36/1.91 (dd+dd, 2H), 1.97 (m, 1H), 0.90 (t, 3H). HRMS calculated for C₂₆H₃₀ClNO₃: 439.1914; found 440.1983 (M+H).

### Preparation 10b methyl (1r,4R)-4-(3-chloroanilino)-2′-[(2R)-3-hydroxy-2-methylpropyl]-2′,3′-dihydrospiro[cyclohexane-1,1′-indene]-4-carboxylate

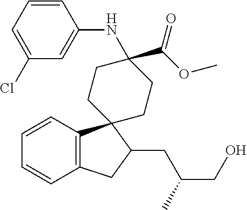

Using General procedure 19 and Preparation 10a as the appropriate indene, Preparation 10b was obtained as a mixture of diastereoisomers. HRMS calculated for C₂₆H₃₂ClNO₃: 441.2071; found 442.2147 and 442.2133 (M+H).

### Preparation 10b1 methyl (1r,2′S,4S)-4-(3-chloroanilino)-2′-[(2R)-3-hydroxy-2-methylpropyl]-2′,3′-dihydrospiro[cyclohexane-1,1′-indene]-4-carboxylate

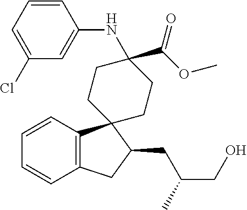 [SMILES: COC(=O)[C@]1(Nc2cccc(Cl)c2)CC[C@@]2(CC1)c1ccccc1C[C@@H]2C[C@@H](C)CO]

and

### Preparation 10b2 methyl (1r,2′R,4R)-4-(3-chloroanilino)-2′-[(2R)-3-hydroxy-2-methylpropyl]-2′,3′-dihydrospiro[cyclohexane-1,1′-indene]-4-carboxylate

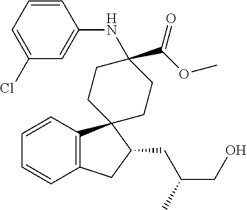 [SMILES: COC(=O)[C@]1(Nc2cccc(Cl)c2)CC[C@@]2(CC1)c1ccccc1C[C@H]2C[C@@H](C)CO]

The diastereoisomers of Preparation 10b were separated by chiral chromatography. Column: AD, 100×500 mm, 20 μm, Eluents: 15:85 iPrOH/heptane. The diastereoisomer eluting earlier was collected as Preparation 10b2. ¹H NMR (500 MHz, DMSO-d₆) δ ppm: 7.37 (d, 1H), 7.20 (d, 1H), 7.15 (t, 1H), 7.13 (t, 1H), 7.06 (t, 1H), 6.59 (dd, 1H), 6.56 (dd, 1H), 6.45 (dd, 1H), 6.30 (s, 1H), 4.39 (br s, 1H), 3.64 (s, 3H), 3.42/3.20 (dd+dd, 2H), 2.96/2.53 (dd+dd, 2H), 2.43/1.99/1.88/1.45 (td+dt/td+dt, 4H), 2.08/1.90/1.71/1.34 (t+d/t+t, 4H), 2.07 (m, 1H), 1.61 (m, 1H), 1.45/1.02 (t+t, 2H), 0.91 (d, 3H). HRMS calculated for C₂₆H₃₂ClNO₃: 441.2071; found 442.2143 (M+H).

The diastereoisomer eluting later was collected as Preparation 10b1. ¹H NMR (500 MHz, DMSO-d₆) δ ppm: 7.36 (d, 1H), 7.20 (d, 1H), 7.15 (t, 1H), 7.13 (t, 1H), 7.06 (t, 1H), 6.59 (dd, 1H), 6.56 (dd, 1H), 6.45 (dd, 1H), 6.31 (s, 1H), 4.45 (t, 1H), 3.64 (s, 3H), 3.22 (t, 2H), 2.93/2.51 (dd+dd, 2H), 2.44/1.97/1.88/1.44 (td+dt/td+dt, 4H), 2.12/1.89/1.71/1.36 (t+d/t+d, 4H), 2.07 (m, 1H), 1.56 (m, 1H), 1.35/1.07 (t+t, 2H), 0.85 (d, 3H). HRMS calculated for C₂₆H₃₂ClNO₃: 441.2071; found 442.2139 (M+H).

### Preparation 11a

### Preparation 11aA 5-(bromomethyl)-2,2-dimethyl-1,3-dioxane

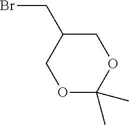

To a stirred solution of (2,2-dimethyl-1,3-dioxan-5-yl)methanol (3.07 g, 21 mmol, 1 eq) in DCM (4 mL) and pyridine (2 mL) was added CBr₄ (3.24 mL, 31.9 mmol, 1.52 eq), followed by PPh₃ (5.51 g, 21 mmol, 1 eq) in DCM (5 mL) over 1.5 h. The mixture was stirred at rt for 18 h, then filtered and concentrated in vacuo. Purification by automated flash column chromatography (CombiFlash Rf, 80 g RediSep™ silica cartridge) eluting with a gradient of 0-100% DCM in heptane afforded Preparation 11aA as a cream oil (1.96 g, 9.36 mmol, 45%). ¹H NMR (400 MHz, DMSO-d₆) δ ppm: 3.99-3.90 (m, 2H), 3.73-3.63 (m, 2H), 3.58 (d, J=7.2 Hz, 2H), 2.00-1.86 (m, 1H), 1.36-1.30 (m, 6H).

### Preparation 11aB bromido[(2,2-dimethyl-1,3-dioxan-5-yl)methyl]zinc

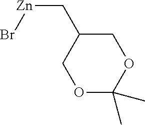

Using General procedure 4 and Preparation 11aA as the appropriate bromo compound, Preparation 11aB was obtained.

### Preparation 11a methyl (1r,4r)-4-(3-chloroanilino)-2′-[3-hydroxy-2-(hydroxymethyl)propyl]spiro[cyclohexane-1,1′-indene]-4-carboxylate

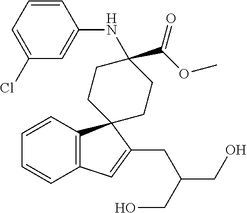 [SMILES: COC(=O)[C@]1(Nc2cccc(Cl)c2)CC[C@]2(CC1)C(CC(CO)CO)=Cc1ccccc12]

To an oven-dried flask was added Preparation 6c (112 mg, 0.25 mmol, 1 eq) and AtaPhos (4 mg, 5 μmol, 0.02 eq) in THE (3 mL). 1-methylimidazole (40 μL, 0.5 mmol, 2 eq) was added followed by Preparation 11aB (1 mL, 0.5 M, 0.5 mmol, 2 eq) and then stirred at rt for 18 h. The mixture was filtered through celite, washed with DCM and the organic phase was washed with 1 M aq. HCl solution, brine, dried (MgSO₄) and concentrated in vacuo. The residue was taken up in MeOH (10 mL) and PTSA (4 μL, 0.04 mmol, 0.2 eq) was added and the mixture was stirred at rt for 1 h, then neutralized with K₂CO₃. The mixture was partitioned between DCM and sat. aq. NaHCO₃ solution. The organic phase was washed with brine, dried (MgSO₄) and concentrated in vacuo. Purification by automated flash column chromatography (CombiFlash Rf, 12 g RediSep™ silica cartridge) eluting with a gradient of 0-10% MeOH in DCM afforded Preparation 11a as a white solid (63 mg, 0.14 mmol, 55%). LRMS calculated for C₂₆H₃₀ClNO₄: 455; found 456 (M+H). ¹H NMR (400 MHz, DMSO-d₆) δ ppm: 7.68-7.62 (m, 1H), 7.32-7.27 (m, 1H), 7.26-7.20 (m, 1H), 7.14-7.07 (m, 2H), 6.65-6.57 (m, 2H), 6.52-6.51 (m, 2H), 6.44 (s, 1H), 4.44 (t, J=5.1 Hz, 2H), 3.69 (s, 3H), 3.51-3.39 (m, 4H), 2.48-2.35 (m, 2H), 2.26-2.07 (m, 6H), 2.02-1.91 (m, 1H), 0.93-0.83 (m, 2H).

### Preparation 11b methyl (1r,4r)-4-(3-chloroanilino)-2′-[3-hydroxy-2-(hydroxymethyl)propyl]-2′,3′-dihydrospiro[cyclohexane-1,1′-indene]-4-carboxylate

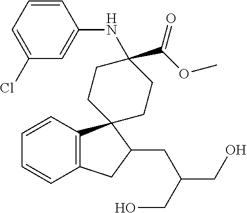

Using General procedure 19 and Preparation 11a as the appropriate indene, Preparation 11b was obtained. LRMS calculated for C₂₆H₃₂ClNO₄: 457; found 458 (M+H).

### Preparation 12a methyl (1s,4s)-4-(3-chloroanilino)-2′-(4-hydroxyphenyl)spiro[cyclohexane-1,1′-indene]-4-carboxylate

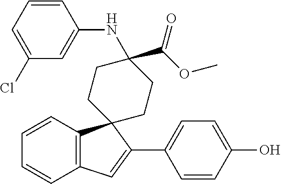 [SMILES: COC(=O)[C@]1(Nc2cccc(Cl)c2)CC[C@]2(CC1)C(c1ccc(O)cc1)=Cc1ccccc12]

Using General procedure 18 and Preparation 6c as the appropriate 2-bromo-indene and (4-hydroxyphenyl)boronic acid as the appropriate boronic acid, Preparation 12a was obtained. ¹H NMR (500 MHz, DMSO-d₆) δ ppm: 9.62 (s, 1H), 7.75 (dm, 1H), 7.68 (m, 2H), 7.38 (dm, 1H), 7.27 (m, 1H), 7.16 (m, 1H), 7.13 (t, 1H), 7.10 (s, 1H), 6.79 (m, 2H), 6.71 (t, 1H), 6.65 (s, 1H), 6.63 (dm, 1H), 6.55 (dm, 1H), 3.71 (s, 3H), 2.50/1.01 (m+m, 4H), 2.44/2.30 (m+m, 4H). HRMS calculated for C₂₈H₂₆ClNO₃: 459.1601; found 460.1667 (M+H).

### Preparation 12b methyl (1s,4s)-4-(3-chloroanilino)-2′-(3-hydroxyphenyl)spiro[cyclohexane-1,1′-indene]-4-carboxylate

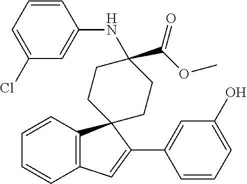 [SMILES: COC(=O)[C@]1(Nc2cccc(Cl)c2)CC[C@]2(CC1)C(c1cccc(O)c1)=Cc1ccccc12]

Using General procedure 18 and Preparation 6c as the appropriate 2-bromo-indene and (3-hydroxyphenyl)boronic acid as the appropriate boronic acid, Preparation 12b was obtained. ¹H NMR (500 MHz, DMSO-d₆) δ ppm: 9.41 (s, 1H), 7.80-7.16 (m, 4H), 7.19 (m, 1H), 7.18 (m, 1H), 7.10 (t, 1H), 7.10 (s, 1H), 7.10 (m, 1H), 6.75 (dm, 1H), 6.69 (t, 1H), 6.60 (dm, 1H), 6.56 (s, 1H), 6.51 (dm, 1H), 3.70 (s, 3H), 2.50/1.04 (m+m, 4H), 2.44/2.28 (m+m, 4H). HRMS calculated for C₂₈H₂₆ClNO₃: 459.1601; found 460.1675 (M+H).

### Preparation 12c methyl (1r,4r)-4-(3-chloroanilino)-2′-(3-hydroxyphenyl)-2′,3′-dihydrospiro[cyclohexane-1,1′-indene]-4-carboxylate, enantiomer 1

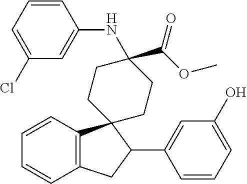

Using General procedure 19 and Preparation 12b as the appropriate indene and using AcOH instead of EtOAc, a racemate was obtained. The enantiomers were separated by chiral chromatography. Column: IA, 100×500 mm, 20 μm, eluents: heptane/THF/iPrOH. The enantiomer eluting earlier was collected as Preparation 12c. ¹H NMR (500 MHz, DMSO-d₆) δ ppm: 9.17 (s, 1H), 7.31-7.17 (m, 4H), 7.02 (t, 1H), 6.98 (t, 1H), 6.56 (dm, 1H), 6.54 (dm, 1H), 6.51 (t, 1H), 6.46 (dm, 1H), 6.40 (t, 1H), 6.39 (dm, 1H), 6.17 (s, 1H), 3.62 (s, 3H), 3.36/2.92 (dd, 2H), 3.32 (dd, 1H), 2.44-1.33 (m, 8H). HRMS calculated for C₂₈H₂₈ClNO₃: 461.1758; found 462.1833 (M+H).

### Preparation 13a

### Preparation 13aAA methyl (1s,4s)-2′-bromo-4-[(3-chlorophenyl)(trifluoroacetyl)amino]spiro[cyclohexane-1,1′-indene]-4-carboxylate

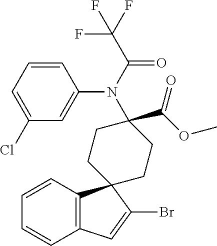 [SMILES: COC(=O)[C@]1(N(C(=O)C(F)(F)F)c2cccc(Cl)c2)CC[C@]2(CC1)C(Br)=Cc1ccccc12]

Preparation 6c (112 g, 251 mmol) was dissolved in 2-Me-THF (564 mL). TEA (175 mL, 1254 mmol) and DMAP (3.06 g, 25.1 mmol) were added to the mixture and cooled to 0° C. TFAA (697 mL, 5013 mmol) was added dropwise at 0° C. (keeping the temperature of the reaction mixture below 10° C.), then it was stirred at 50° C. for 18 h. Then it was cooled to 0° C. and stirred at 0° C. for 2 h. The precipitate was filtered, taken up in DIPE (200 mL) and sonicated. The precipitate was filtered, washed with DIPE and dried to obtain Preparation 13aAA. ¹H NMR (500 MHz, DMSO-d₆) δ ppm: 7.81 (m, 1H), 7.68 (m, 2H), 7.62 (t, 1H), 7.49 (dm, 1H), 7.32 (dm, 1H), 7.27 (m, 1H), 7.23 (m, 1H), 7.02 (s, 1H), 3.84 (s, 3H), 2.55-0.93 (m, 8H). HRMS calculated for C₂₄H₂₀BrClF₃NO₃: 541.0267; found 542.0328 (M+H).

### Preparation 13aA methyl (1s,4s)-2′-bromo-4-[(3-chlorophenyl)(trifluoroacetyl)amino]-6′-formylspiro[cyclohexane-1,1′-indene]-4-carboxylate

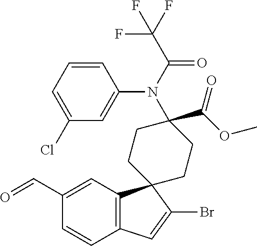 [SMILES: COC(=O)[C@]1(N(C(=O)C(F)(F)F)c2cccc(Cl)c2)CC[C@]2(CC1)C(Br)=Cc1ccc(C=O)cc12]

Using General procedure 26 and Preparation 13aAA as the appropriate indene, Preparation 13aA was obtained. ¹H NMR (500 MHz, DMSO-d₆) δ ppm: 10.00 (d, 1H), 7.97 (br s, 1H), 7.88 (dd, 1H), 7.82 (m, 1H), 7.73-7.6 (m, 3H), 7.55 (d, 1H), 7.19 (s, 1H), 3.87 (s, 3H), 2.58-1.40 (m, 8H). HRMS calculated for C₂₅H₂₀BrClF₃NO₄: 569.0216; found 587.0559 (M+NH₄).

### Preparation 13aB methyl (1r,4R)-4-[(3-chlorophenyl)(trifluoroacetyl)amino]-6′-formyl-2′-{(2R)-3-[(4-methoxyphenyl)methoxy]-2-methylpropyl}spiro[cyclohexane-1,1′-indene]-4-carboxylate

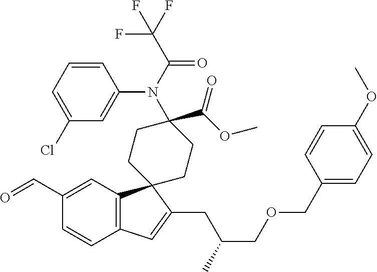 [SMILES: COc1ccc(COC[C@H](C)CC2=Cc3ccc(C=O)cc3[C@]23CC[C@](C(=O)OC)(N(C(=O)C(F)(F)F)c2cccc(Cl)c2)CC3)cc1]

Using General procedure 27b and Preparation 13aA as the appropriate 2-bromo-indene derivative and Preparation 3d as the appropriate Zn reagent, Preparation 13aB was obtained. ¹H NMR (500 MHz, DMSO-d₆) δ ppm: 9.95 (s, 1H), 7.99 (d, 1H), 7.93-7.48 (m, 4H), 7.81 (d, 1H), 7.45 (dd, 1H), 7.25/7.24 (m, 2H), 6.89 (m, 2H), 6.58/6.57 (s, 1H), 4.47-4.33 (d+d, 2H), 3.86 (s, 3H), 3.72 (s, 3H), 3.42-3.25 (m, 2H), 2.66-1.02 (m, 11H), 0.95/0.93 (d, 3H). HRMS calculated for C₃₇H₃₇ClF₃NO₆: 683.2261; found 706.21591 (M+Na).

### Preparation 13aC (1r,4R)-4-(3-chloroanilino)-6′-formyl-2′-{(2R)-3-[(4-methoxyphenyl)methoxy]-2-methylpropyl}spiro[cyclohexane-1,1′-indene]-4-carboxylic acid

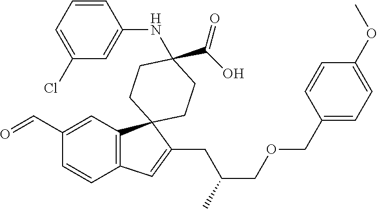 [SMILES: COc1ccc(COC[C@H](C)CC2=Cc3ccc(C=O)cc3[C@]23CC[C@@](Nc2cccc(Cl)c2)(C(=O)O)CC3)cc1]

Using General Procedure 33a and Preparation 13aB as the appropriate ester, Preparation 13aC was obtained. ¹H NMR (500 MHz, DMSO-d₆) δ ppm: 12.83 (br s, 1H), 9.97 (s, 1H), 8.12 (br s, 1H), 7.84 (dd, 1H), 7.49 (d, 1H), 7.22 (dm, 2H), 7.10 (t, 1H), 6.85 (dm, 2H), 6.66 (t, 1H), 6.61 (s, 1H), 6.58 (m, 2H), 6.36 (br s, 1H), 4.40/4.37 (d+d, 2H), 3.72 (s, 3H), 3.34/3.30 (dd+dd, 2H), 2.46-2.01 (m, 8H), 2.42/2.06 (dd+dd, 2H), 2.20 (m, 1H), 0.94 (d, 3H). HRMS calculated for C₃₄H₃₆ClNO₅: 573.2282; found 574.2344 (M+H).

### Preparation 13aD methyl (1r,4R)-4-(3-chloroanilino)-6′-formyl-2′-{(2R)-3-[(4-methoxyphenyl)methoxy]-2-methylpropyl}spiro[cyclohexane-1,1′-indene]-4-carboxylate

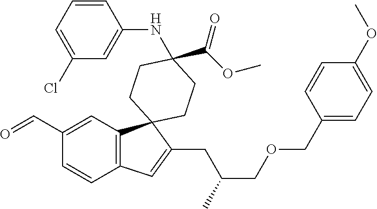 [SMILES: COc1ccc(COC[C@H](C)CC2=Cc3ccc(C=O)cc3[C@]23CC[C@@](Nc2cccc(Cl)c2)(C(=O)OC)CC3)cc1]

Using General procedure 17a and Preparation 13aC as the appropriate amino acid, Preparation 13aD was obtained. ¹H NMR (500 MHz, DMSO-d₆) δ ppm: 9.98 (s, 1H), 8.12 (s, 1H), 7.83 (dd, 1H), 7.48 (d, 1H), 7.21 (d, 2H), 7.10 (t, 1H), 6.85 (d, 2H), 6.66 (dd, 1H), 6.60 (dd, 1H), 6.60 (s, 1H), 6.49 (dd, 1H), 6.45 (s, 1H), 4.40/4.36 (d+d, 2H), 3.72 (s, 3H), 3.71 (s, 3H), 3.33/3.29 (dd+dd, 2H), 2.46-0.87 (m, 8H), 2.42/2.05 (dd+dd, 2H), 2.19 (m, 1H), 0.94 (d, 3H). HRMS calculated for C₃₅H₃₈ClNO₅: 587.2438; found 588.2521 (M+H).

### Preparation 13aE methyl (1r,4R)-4-(3-chloroanilino)-6′-formyl-2′-[(2R)-3-hydroxy-2-methylpropyl]spiro[cyclohexane-1,1′-indene]-4-carboxylate

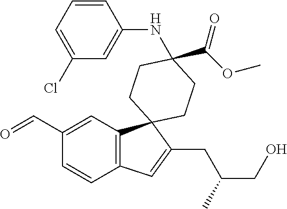 [SMILES: COC(=O)[C@]1(Nc2cccc(Cl)c2)CC[C@]2(CC1)C(C[C@@H](C)CO)=Cc1ccc(C=O)cc12]

Using General procedure 28a and Preparation 13aD as the appropriate PMB derivative, Preparation 13aE was obtained. ¹H NMR (500 MHz, DMSO-d₆) δ ppm: 9.98 (s, 1H), 8.13 (br s, 1H), 7.84 (dd, 1H), 7.51 (d, 1H), 7.10 (t, 1H), 6.65 (t, 1H), 6.63 (s, 1H), 6.60 (dm, 1H), 6.49 (dm, 1H), 6.45 (s, 1H), 4.59 (t, 1H), 3.71 (s, 3H), 3.33 (m, 2H), 2.47-0.87 (m, 8H), 2.43/1.97 (m+m, 2H), 1.99 (m, 1H), 0.90 (d, 3H). HRMS calculated for C₂₇H₃₀ClNO₄: 467.1863; found 468.1924 (M+H).

### Preparation 13aF methyl (1r,4R)-4-(3-chloroanilino)-6′-(1,3-dioxan-2-yl)-2′-[(2R)-3-hydroxy-2-methylpropyl]spiro[cyclohexane-1,1′-indene]-4-carboxylate

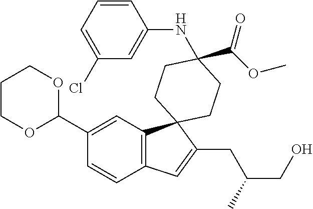 [SMILES: COC(=O)[C@]1(Nc2cccc(Cl)c2)CC[C@]2(CC1)C(C[C@@H](C)CO)=Cc1ccc(C3OCCCO3)cc12]

Preparation 13aE (1.39 g, 2.97 mmol) was dissolved in toluene (44.5 mL). Propane-1,3-diol (2.15 mL, 29.7 mmol) and PPTS (60 mg, 0.24 mmol) were added and the mixture was stirred at reflux temperature for 1 h using a Dean-Stark apparatus. Then it was concentrated under reduced pressure and purified via flash chromatography using heptane and EtOAc as eluents to obtain Preparation 13aF. ¹H NMR (500 MHz, DMSO-d₆) δ ppm: 7.73 (br s, 1H), 7.26 (dd, 1H), 7.24 (d, 1H), 7.10 (t, 1H), 6.63 (t, 1H), 6.59 (dm, 1H), 6.47 (dm, 1H), 6.46 (s, 1H), 6.41 (s, 1H), 5.52 (s, 1H), 4.54 (t, 1H), 4.15/3.96 (dm+tm, 4H), 3.71 (s, 3H), 3.34/3.29 (m+m, 2H), 2.44-0.80 (m, 8H), 2.35/1.90 (m+m, 2H), 2.00/1.45 (m+dm, 2H), 1.96 (m, 1H), 0.89 (d, 3H). HRMS calculated for C₃₀H₃₆ClNO₅: 525.2282; found 526.23491 (M+H).

### Preparation 13aG methyl (1r,2′R,4R)-4-(3-chloroanilino)-6′-(1,3-dioxan-2-yl)-2′-[(2R)-3-hydroxy-2-methylpropyl]-2′,3′-dihydrospiro[cyclohexane-1,1′-indene]-4-carboxylate

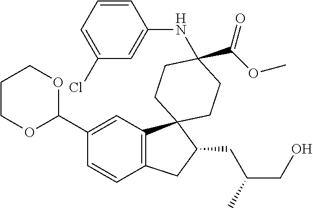 [SMILES: COC(=O)[C@]1(Nc2cccc(Cl)c2)CC[C@@]2(CC1)c1cc(C3OCCCO3)ccc1C[C@H]2C[C@@H](C)CO]

and

### Preparation 13aH methyl (1r,2′S,4S)-4-(3-chloroanilino)-6′-(1,3-dioxan-2-yl)-2′-[(2R)-3-hydroxy-2-methylpropyl]-2′,3′-dihydrospiro[cyclohexane-1,1′-indene]-4-carboxylate

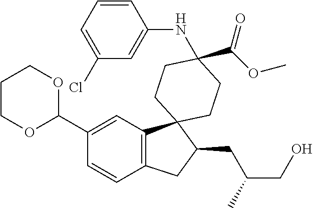 [SMILES: COC(=O)[C@]1(Nc2cccc(Cl)c2)CC[C@@]2(CC1)c1cc(C3OCCCO3)ccc1C[C@@H]2C[C@@H](C)CO]

Using General procedure 19 and Preparation 13aF as the appropriate indene, a mixture of diastereoisomers was obtained. The diastereoisomers were separated by chiral chromatography. Column: AD, 100×500 mm, 20 μm, Eluents: 15:85 EtOH/heptane. The diastereoisomer eluting earlier was collected as Preparation 13aG. ¹H NMR (500 MHz, DMSO-d₆) δ ppm: 7.35 (s, 1H), 7.17 (m, 2H), 7.07 (t, 1H), 6.60 (t, 1H), 6.57 (dd, 1H), 6.46 (dd, 1H), 6.29 (s, 1H), 5.48 (s, 1H), 4.39 (t, 1H), 4.13/3.93 (dd+dd, 4H), 3.65 (s, 3H), 3.42/3.19 (m+m, 2H), 2.95/2.53 (dd+dd, 2H), 2.41-1.36 (m, 8H), 2.14 (m, 1H), 1.99/1.44 (m, 2H), 1.60 (m, 1H), 1.41/0.94 (m+m, 2H), 0.89 (d, 3H). HRMS calculated for C₃₀H₃₈ClNO₅: 527.2438; found 528.2505 (M+H).

The diastereoisomer eluting later was collected as Preparation 13aH. ¹H NMR (500 MHz, DMSO-d₆) δ ppm: 7.34 (s, 1H), 7.17 (m, 2H), 7.07 (t, 1H), 6.60 (t, 1H), 6.56 (dd, 1H), 6.46 (dd, 1H), 6.31 (s, 1H), 5.48 (s, 1H), 4.43 (t, 1H), 4.14/3.93 (dd+dd, 4H), 3.66 (s, 3H), 3.20 (m, 2H), 2.94/2.49 (dd+dd, 2H), 2.42-1.37 (m, 8H), 2.13 (m, 1H), 1.99/1.44 (m+m, 2H), 1.56 (m, 1H), 1.25/1.03 (m+m, 2H), 0.85 (d, 3H). HRMS calculated for C₃₀H₃₈ClNO₅: 527.2438; found 528.2507 (M+H).

### Preparation 13aI methyl (1r,2′S,4S)-4-(3-chloroanilino)-6′-(1,3-dioxan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

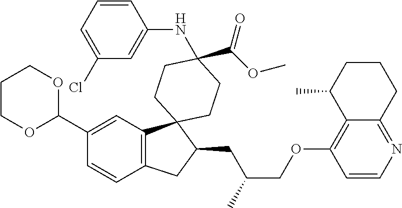 [SMILES: COC(=O)[C@]1(Nc2cccc(Cl)c2)CC[C@@]2(CC1)c1cc(C3OCCCO3)ccc1C[C@@H]2C[C@@H](C)COc1ccnc2c1[C@H](C)CCC2]

Using General procedure 30a and Preparation 13aH as the appropriate indane and Preparation 2a1 as the appropriate aryl-alcohol, Preparation 13aI was obtained. ¹H NMR (500 MHz, DMSO-d₆) δ ppm: 8.13 (d, 1H), 7.36 (d, 1H), 7.18 (dd, 1H), 7.17 (d, 1H), 7.05 (t, 1H), 6.75 (d, 1H), 6.59 (t, 1H), 6.56 (dm, 1H), 6.45 (dm, 1H), 6.33 (s, 1H), 5.48 (s, 1H), 4.17-3.88 (m, 4H), 3.89/3.83 (dd+dd, 2H), 3.66 (s, 3H), 3.03 (m, 1H), 3.01/2.53 (dd+dd, 2H), 2.76/2.64 (m+m, 2H), 2.50-1.36 (m, 14H), 2.20 (m, 1H), 1.99 (m, 1H), 1.42/1.31 (m+m, 2H), 1.05 (d, 3H), 1.02 (d, 3H). HRMS calculated for C₄₀H₄₉ClN₂O₅: 672.333; found 673.3389 (M+H).

### Preparation 13a methyl (1r,2′S,4S)-4-(3-chloroanilino)-6′-formyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

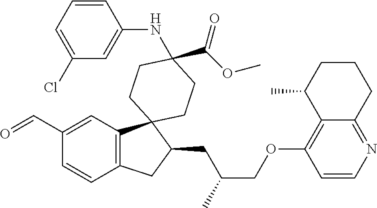 [SMILES: COC(=O)[C@]1(Nc2cccc(Cl)c2)CC[C@@]2(CC1)c1cc(C=O)ccc1C[C@@H]2C[C@@H](C)COc1ccnc2c1[C@H](C)CCC2]

Preparation 13aI (430 mg, 0.64 mmol) was dissolved in acetone (4.8 mL), then 2 M aq. HCl solution (3.2 mL) was added. The mixture was stirred at 45° C. until no further conversion was observed. The mixture was allowed to cool to rt. The pH was adjusted to 7 with sat. aq. NaHCO₃ solution and acetone was removed under reduced pressure. The mixture was extracted with EtOAc and the combined organic layers were dried over Na₂SO₄, filtered and the filtrate was concentrated under reduced pressure. The crude product was purified via flash chromatography using heptane and EtOAc as eluents to obtain Preparation 13a. ¹H NMR (500 MHz, DMSO-d₆) δ ppm: 9.98 (s, 1H), 8.14 (d, 1H), 7.85 (br, 1H), 7.75 (dd, 1H), 7.45 (d, 1H), 7.06 (t, 1H), 6.76 (d, 1H), 6.60 (t, 1H), 6.56 (dd, 1H), 6.46 (dd, 1H), 6.35 (s, 1H), 3.87 (m, 2H), 3.66 (s, 3H), 3.11/2.63 (dd+dd, 2H), 3.02 (m, 1H), 2.75/2.63 (m+m, 2H), 2.46-1.46 (m, 8H), 2.24 (m, 1H), 2.01 (m, 1H), 1.77/1.70 (m+m, 2H), 1.65/1.58 (m+m, 2H), 1.46/1.34 (m+m, 2H), 1.05 (d, 3H), 1.00 (d, 3H). HRMS calculated for C₃₇H₄₃ClN₂O₄: 614.2911; found 615.29814 (M+H).

### Preparation 13b

### Preparation 13bA methyl (1r,4R)-4-[(3-chlorophenyl)(trifluoroacetyl)amino]-6′-formyl-2′-[(2R)-3-hydroxy-2-methylpropyl]spiro[cyclohexane-1,1′-indene]-4-carboxylate

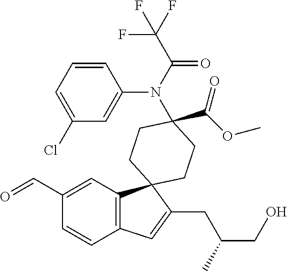 [SMILES: COC(=O)[C@]1(N(C(=O)C(F)(F)F)c2cccc(Cl)c2)CC[C@]2(CC1)C(C[C@@H](C)CO)=Cc1ccc(C=O)cc12]

Using General procedure 28a and Preparation 13aB as the appropriate PMB derivative, Preparation 13bA was obtained as a white solid. LRMS calculated for C₂₉H₂₉ClF₃NO₅: 563; found: 564 (M+H).

### Preparation 13bB methyl (1r,4R)-4-[(3-chlorophenyl)(trifluoroacetyl)amino]-6′-(1,3-dioxan-2-yl)-2′-[(2R)-3-hydroxy-2-methylpropyl]spiro[cyclohexane-1,1′-indene]-4-carboxylate

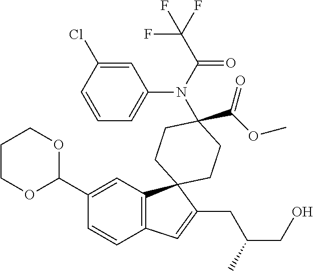 [SMILES: COC(=O)[C@]1(N(C(=O)C(F)(F)F)c2cccc(Cl)c2)CC[C@]2(CC1)C(C[C@@H](C)CO)=Cc1ccc(C3OCCCO3)cc12]

To a solution of Preparation 13bA (26.58 g, 47.13 mmol, 1 eq) in toluene (650 mL) was added propane-1,3-diol (34.2 mL, 471 mmol, 10 eq) and PPTS (0.95 g, 3.77 mmol, 0.08 eq). The mixture was heated at reflux for 1 h using Dean-Stark apparatus (pre-filled with toluene) and then allowed to cool to rt. The mixture was partitioned between DCM and water, and the organic phase was washed with brine, dried (PTFE phase separator) and concentrated in vacuo. Purification by automated flash chromatography (CombiFlash Rf, 330 g RediSep™ silica cartridge) eluting with a gradient of 0-50% EtOAc in heptane afforded Preparation 13bB as a white foam (26.4 g, 42.4 mmol, 90%). LRMS calculated for C₃₂H₃₅ClF₃NO₆: 621; found: 622 (M+H).

### Preparation 13bC methyl (1r,2′R,4R)-4-[(3-chlorophenyl)(trifluoroacetyl)amino]-6′-(1,3-dioxan-2-yl)-2′-[(2R)-3-hydroxy-2-methylpropyl]-2′,3′-dihydrospiro[cyclohexane-1,1′-indene]-4-carboxylate

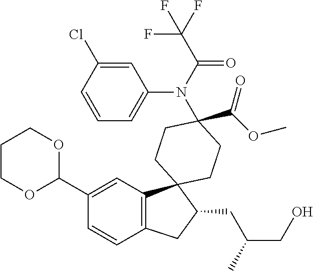 [SMILES: COC(=O)[C@]1(N(C(=O)C(F)(F)F)c2cccc(Cl)c2)CC[C@@]2(CC1)c1cc(C3OCCCO3)ccc1C[C@H]2C[C@@H](C)CO]

and

### Preparation 13bD methyl (1r,2′S,4S)-4-[(3-chlorophenyl)(trifluoroacetyl)amino]-6′-(1,3-dioxan-2-yl)-2′-[(2R)-3-hydroxy-2-methylpropyl]-2′,3′-dihydrospiro[cyclohexane-1,1′-indene]-4-carboxylate

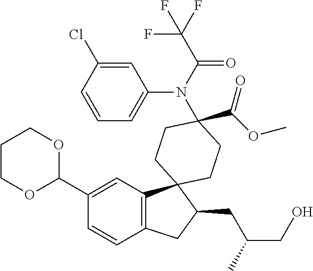 [SMILES: COC(=O)[C@]1(N(C(=O)C(F)(F)F)c2cccc(Cl)c2)CC[C@@]2(CC1)c1cc(C3OCCCO3)ccc1C[C@@H]2C[C@@H](C)CO]

Using General procedure 19 and Preparation 13bB as the appropriate indene, a mixture of distereoisomers was obtained. They were purified and separated by automated flash chromatography (CombiFlash Rf, 330 g RediSep™ silica cartridge) eluting with a gradient of 0-45% EtOAc in heptane. The diastereoisomer eluting earlier was collected as Preparation 13bC, isolated as a white solid. LRMS calculated for C₃₂H₃₇ClF₃NO₆: 623; found: 624 (M+H). ¹H NMR (400 MHz, DMSO-d₆) δ ppm: 7.70-7.50 (m, 4H), 7.17-7.05 (m, 3H), 5.46 (s, 1H), 4.43-4.35 (m, 1H), 4.18-4.09 (m, 2H), 3.97-3.87 (m, 2H), 3.79/3.78 (s, 3H), 3.44-3.36 (m, 1H), 3.15-2.92 (m, 2H), 2.54-2.46 (m, 1H), 2.30-1.93 (m, 5H), 1.73-1.40 (m, 7H), 1.16-1.04 (m, 1H), 0.85-0.77 (m, 3H), 0.68-0.57 (m, 1H).

The diastereoisomer eluting later was collected as Preparation 13bD, isolated as a white solid.

LRMS calculated for C₃₂H₃₇ClF₃NO₆: 623; found: 624 (M+H). ¹H NMR (400 MHz, DMSO-d₆) δ ppm: 7.76-7.44 (m, 4H), 7.16-7.00 (m, 3H), 5.46 (s, 1H), 4.39-4.32 (m, 1H), 4.17-4.09 (m, 2H), 3.97-3.87 (m, 2H), 3.79/3.79 (s, 3H), 3.17-2.90 (m, 3H), 2.54-1.36 (m, 13H), 1.02-0.52 (m, 5H).

### Preparation 13bE methyl (1r,2′S,4S)-4-[(3-chlorophenyl)(trifluoroacetyl)amino]-6′-(1,3-dioxan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

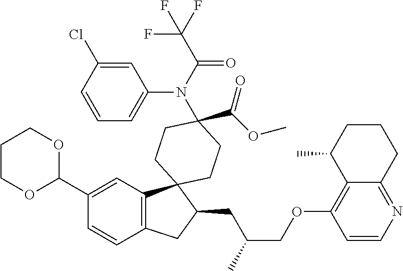 [SMILES: COC(=O)[C@]1(N(C(=O)C(F)(F)F)c2cccc(Cl)c2)CC[C@@]2(CC1)c1cc(C3OCCCO3)ccc1C[C@@H]2C[C@@H](C)COc1ccnc2c1[C@H](C)CCC2]

Using General procedure 30a and Preparation 13bD as the appropriate indene and Preparation 2a1 as the appropriate alcohol, Preparation 13bE was obtained as a white solid. LRMS calculated for C₄₂H₄₈ClF₃N₂O₆: 768; found: 769 (M+H).

### Preparation 13b methyl (1r,2′S,4S)-4-[(3-chlorophenyl)(trifluoroacetyl)amino]-6′-formyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

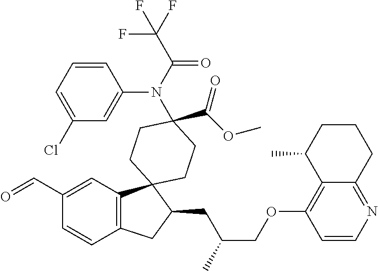 [SMILES: COC(=O)[C@]1(N(C(=O)C(F)(F)F)c2cccc(Cl)c2)CC[C@@]2(CC1)c1cc(C=O)ccc1C[C@@H]2C[C@@H](C)COc1ccnc2c1[C@H](C)CCC2]

A solution of Preparation 13bE (6.04 g, 7.85 mmol, 1 eq) in a mixture of AcOH (24.3 mL, 424 mmol, 54 eq) and water (25 mL) was heated at 90° C. for 1 h. The mixture was allowed to cool to rt and partitioned between EtOAc and water. The phases were separated, and the organic phase was washed with sat. aq. NaHCO₃ solution, brine, dried (MgSO₄) and concentrated in vacuo. Purification by automated flash chromatography (CombiFlash Rf, 120 g RediSep™ silica cartridge) eluting with a gradient of 0-100% EtOAc in heptane afforded Preparation 13b as a white foam (4.8 g, 6.76 mmol, 86%). LRMS calculated for C₃₉H₄₂ClF₃N₂O₅: 710; found: 711 (M+H).

### Preparation 13c methyl (1r,2′S,4S)-4-(3-chloroanilino)-6′-(hydroxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

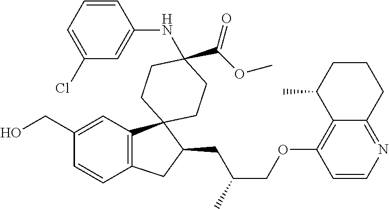 [SMILES: COC(=O)[C@]1(Nc2cccc(Cl)c2)CC[C@@]2(CC1)c1cc(CO)ccc1C[C@@H]2C[C@@H](C)COc1ccnc2c1[C@H](C)CCC2]

Using General procedure 36 and Preparation 13a as the appropriate formyl derivative, Preparation 13c was obtained. ¹H NMR (500 MHz, DMSO-d₆) δ ppm: 8.14 (d, 1H), 7.32 (br s, 1H), 7.13 (d, 1H), 7.08 (dd, 1H), 7.05 (t, 1H), 6.76 (d, 1H), 6.60 (t, 1H), 6.56 (dm, 1H), 6.46 (dm, 1H), 6.32 (s, 1H), 5.12 (t, 1H), 4.46 (d, 2H), 3.90/3.84 (dd+dd, 2H), 3.65 (s, 3H), 3.05 (m, 1H), 2.97/2.50 (dd+dd, 2H), 2.76/2.67 (m+m, 2H), 2.50-1.36 (m, 8H), 2.15 (m, 1H), 2.00 (m, 1H), 1.84-1.66 (m, 2H), 1.66/1.60 (m+m, 2H), 1.45/1.33 (m+m, 2H), 1.05 (d, 3H), 1.04 (d, 3H). HRMS calculated for C₃₇H₄₅ClN₂O₄: 616.3068; found: 617.3141 (M+H).

### Preparation 13d

### Preparation 13dA methyl (1r,2′R,4R)-4-(3-chloroanilino)-6′-(1,3-dioxan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

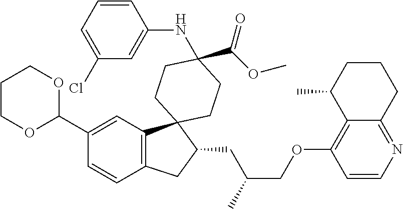 [SMILES: COC(=O)[C@]1(Nc2cccc(Cl)c2)CC[C@@]2(CC1)c1cc(C3OCCCO3)ccc1C[C@H]2C[C@@H](C)COc1ccnc2c1[C@H](C)CCC2]

Using General procedure 30a and Preparation 13aG as the appropriate indane and Preparation 2a1 as the appropriate alcohol, Preparation 13dA was obtained. ¹H NMR (500 MHz, DMSO-d₆) δ ppm: 8.13 (d, 1H), 7.39 (d, 1H), 7.18 (dd, 1H), 7.17 (d, 1H), 7.05 (t, 1H), 6.78 (d, 1H), 6.58 (t, 1H), 6.57 (dm, 1H), 6.41 (dm, 1H), 6.23 (s, 1H), 5.47 (s, 1H), 4.17-3.88 (m, 4H), 4.00/3.87 (dd+dd, 2H), 3.64 (s, 3H), 3.00 (m, 1H), 3.00/2.58 (dd+dd, 2H), 2.73/2.59 (m+m, 2H), 2.45-1.28 (m, 14H), 2.13 (m, 1H), 2.06 (m, 1H), 1.66/1.18 (m+m, 2H), 1.08 (d, 3H), 1.08 (d, 3H). HRMS calculated for C₄₀H₄₉ClN₂O₅: 672.3330; found: 673.3408 (M+H).

### Preparation 13 dB methyl (1r,2′R,4R)-4-(3-chloroanilino)-6′-formyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

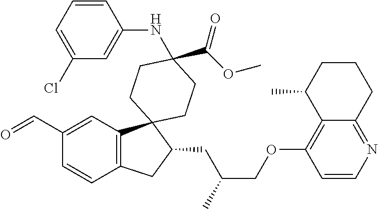 [SMILES: COC(=O)[C@]1(Nc2cccc(Cl)c2)CC[C@@]2(CC1)c1cc(C=O)ccc1C[C@H]2C[C@@H](C)COc1ccnc2c1[C@H](C)CCC2]

Using General procedure 9 and Preparation 13dA as the appropriate acetal, Preparation 13 dB was obtained. ¹H NMR (500 MHz, DMSO-d₆) δ ppm: 9.97 (s, 1H), 8.15 (d, 1H), 7.88 (d, 1H), 7.74 (dd, 1H), 7.45 (d, 1H), 7.06 (t, 1H), 6.77 (d, 1H), 6.59 (t, 1H), 6.57 (dd, 1H), 6.43 (dd, 1H), 6.28 (s, 1H), 4.01/3.88 (dd+dd, 2H), 3.66 (s, 3H), 3.12/2.70 (dd+dd, 2H), 3.07 (m, 1H), 2.72/2.60 (m+m, 2H), 2.44-1.40 (m, 8H), 2.15 (m, 1H), 2.07 (m, 1H), 1.76/1.64 (m+m, 2H), 1.70/1.23 (m+m, 2H), 1.48 (m, 2H), 1.10 (d, 3H), 1.08 (d, 3H). HRMS calculated for C₃₇H₄₃ClN₂O₄: 614.2911; found: 615.2981 (M+H).

### Preparation 13d methyl (1r,2′R,4R)-4-(3-chloroanilino)-6′-(hydroxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

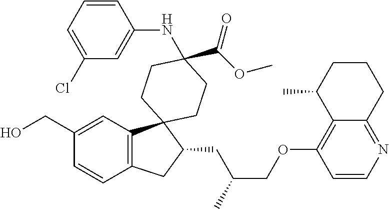 [SMILES: COC(=O)[C@]1(Nc2cccc(Cl)c2)CC[C@@]2(CC1)c1cc(CO)ccc1C[C@H]2C[C@@H](C)COc1ccnc2c1[C@H](C)CCC2]

Using General procedure 36 and Preparation 13 dB as the appropriate formyl derivative, Preparation 13d was obtained. ¹H NMR (500 MHz, DMSO-d₆) δ ppm: 8.13 (d, 1H), 7.34 (br s, 1H), 7.14 (d, 1H), 7.07 (br d, 1H), 7.05 (t, 1H), 6.78 (d, 1H), 6.59 (t, 1H), 6.56 (dm, 1H), 6.42 (dm, 1H), 6.23 (s, 1H), 5.11 (t, 1H), 4.45 (d, 2H), 4.00/3.87 (dd+dd, 2H), 3.64 (s, 3H), 3.00 (m, 1H), 2.97/2.56 (dd+dd, 2H), 2.73/2.60 (m+m, 2H), 2.48-1.32 (m, 8H), 2.10 (br, 1H), 2.07 (m, 1H), 1.76-1.66 (m, 2H), 1.68/1.21 (m+m, 2H), 1.52/1.47 (m+m, 2H), 1.09 (d, 3H), 1.09 (d, 3H). HRMS calculated for C₃₇H₄₅ClN₂O₄: 616.3068; found: 617.3140 (M+H).

### Preparation 14a and Preparation 14b Preparation 14aA methyl (1s,4s)-6′-acetyl-2′-bromo-4-[(3-chlorophenyl)(trifluoroacetyl)amino]spiro[cyclohexane-1,1′-indene]-4-carboxylate

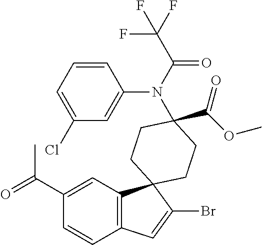 [SMILES: COC(=O)[C@]1(N(C(=O)C(F)(F)F)c2cccc(Cl)c2)CC[C@]2(CC1)C(Br)=Cc1ccc(C(C)=O)cc12]

Using General procedure 23 and Preparation 13aAA as the appropriate indene, Preparation 14aA was obtained. ¹H NMR (500 MHz, DMSO-d₆) δ ppm: 8.10 (d, 1H), 7.94 (dd, 1H), 7.83 (m, 1H), 7.73-7.60 (m, 3H), 7.46 (d, 1H), 7.15 (s, 1H), 3.86 (s, 3H), 2.65-1.28 (m, 8H), 2.60 (s, 3H). HRMS calculated for C₂₆H₂₂BrClF₃NO₄: 583.0373; found 584.0438 (M+H).

### Preparation 14aB methyl (1s,4s)-6′-(acetyloxy)-2′-bromo-4-[(3-chlorophenyl)(trifluoroacetyl)amino]spiro[cyclohexane-1,1′-indene]-4-carboxylate

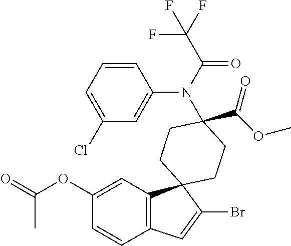 [SMILES: COC(=O)[C@]1(N(C(=O)C(F)(F)F)c2cccc(Cl)c2)CC[C@]2(CC1)C(Br)=Cc1ccc(OC(C)=O)cc12]

Using General procedure 24 and Preparation 14aA as the appropriate indene, Preparation 14aB was obtained. ¹H NMR (500 MHz, DMSO-d₆) δ ppm: 7.81-7.59 (m, 4H), 7.34 (d, 1H), 7.17 (d, 1H), 7.04 (dd, 1H), 7.03 (s, 1H), 3.82 (s, 3H), 2.45-1.44 (m, 8H), 2.30 (s, 3H). HRMS calculated for C₂₆H₂₂BrClF₃NO₅: 599.0322; found 617.0654 (M+NH₄).

### Preparation 14aC methyl (1s,4s)-2′-bromo-4-[(3-chlorophenyl)(trifluoroacetyl)amino]-6′-hydroxyspiro[cyclohexane-1,1′-indene]-4-carboxylate

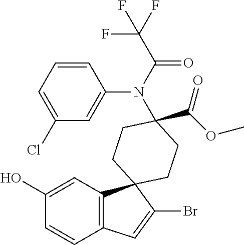 [SMILES: COC(=O)[C@]1(N(C(=O)C(F)(F)F)c2cccc(Cl)c2)CC[C@]2(CC1)C(Br)=Cc1ccc(O)cc12]

Using General procedure 25 and Preparation 14aB as the appropriate indene, Preparation 14aC was obtained. ¹H NMR (500 MHz, DMSO-d₆) δ ppm: 9.53 (s, 1H), 7.77 (br s, 1H), 7.68-7.59 (m, 3H), 7.08 (d, 1H), 6.92 (d, 1H), 6.85 (s, 1H), 6.65 (dd, 1H), 3.84 (s, 3H), 2.40-1.50 (m, 8H). HRMS calculated for C₂₄H₂₀BrClF₃NO₄: 557.0216; found 575.0545 (M+NH₄).

### Preparation 14aD methyl (1s,4s)-2′-bromo-4-[(3-chlorophenyl)(trifluoroacetyl)amino]-6′-(methoxymethoxy)spiro[cyclohexane-1,1′-indene]-4-carboxylate

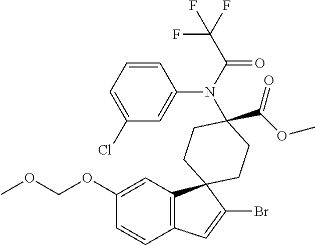

Preparation 14aC (111 g, 199 mmol) was dissolved in DCM (993 mL) and cooled to 0° C. under N₂ atmosphere. DIPEA (138 mL, 795 mmol) and MOM-Cl (60 mL, 795 mmol) were added at 0° C., then the mixture was allowed to warm to rt and stirred overnight. Then it was diluted with water and sat. aq. NaHCO₃ solution and extracted with DCM. The combined organic layers were dried over Na₂SO₄, filtered and the filtrate was concentrated under reduced pressure. The crude product was purified via flash chromatography using heptane and EtOAc as eluents to obtain Preparation 14aD. ¹H NMR (500 MHz, DMSO-d₆) δ ppm: 7.83-7.58 (m, 4H), 7.23 (d, 1H), 7.17 (d, 1H), 6.95 (dd, 1H), 6.94 (s, 1H), 5.19 (s, 2H), 3.83 (s, 3H), 3.40 (s, 3H), 2.55-1.30 (m, 8H). HRMS calculated for C₂₆H₂₄BrClF₃NO₅: 601.0479; found 619.0823 (M+NH₄).

### Preparation 14aE methyl (1r,4R)-4-[(3-chlorophenyl)(trifluoroacetyl)amino]-6′-(methoxymethoxy)-2′-{(2R)-3-[(4-methoxyphenyl)methoxy]-2-methylpropyl}spiro[cyclohexane-1,1′-indene]-4-carboxylate

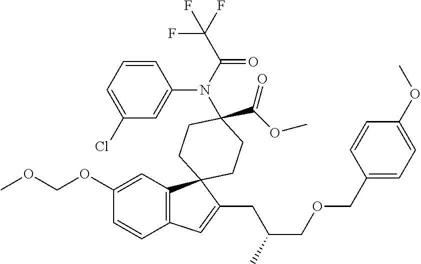 [SMILES: COCOc1ccc2c(c1)[C@]1(CC[C@](C(=O)OC)(N(C(=O)C(F)(F)F)c3cccc(Cl)c3)CC1)C(C[C@@H](C)COCc1ccc(OC)cc1)=C2]

Using General procedure 27b and Preparation 14aD as the appropriate 2-bromo-indene derivative and Preparation 3d as the appropriate Zn reagent, Preparation 14aE was obtained. ¹H NMR (500 MHz, DMSO-d₆) δ ppm: 7.85/7.78 (s/s, 1H), 7.70-7.63 (m, 2H), 7.55/7.50 (t/t, 1H), 7.25/7.23 (d/d, 2H), 7.20 (d, 1H), 7.13/7.12 (d/d, 1H), 6.89 (d, 2H), 6.88 (d, 1H), 6.34/6.33 (s/s, 1H), 5.16 (s, 2H), 4.43/4.41/4.38/4.35 (d+d/d+d, 2H), 3.82 (s, 3H), 3.73 (s, 3H), 3.40 (s, 3H), 3.33/3.28 (dd+dd, 2H), 2.60-1.00 (m, 8H), 2.27/2.17/1.89/1.80 (dd+dd/dd+dd, 2H), 2.10 (m, 1H), 0.93/0.91 (d/d, 3H). HRMS calculated for C₃₈H₄₁ClF₃NO₇: 715.2524; found 733.2882 (M+NH₄).

### Preparation 14aF methyl (1r,2′S,4S)-4-[(3-chlorophenyl)(trifluoroacetyl)amino]-6′-(methoxymethoxy)-2′-{(2R)-3-[(4-methoxyphenyl)methoxy]-2-methylpropyl}-2′,3′-dihydrospiro[cyclohexane-1,1′-indene]-4-carboxylate

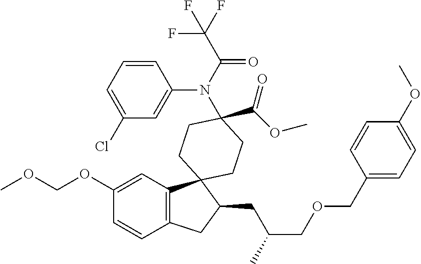 [SMILES: COCOc1ccc2c(c1)[C@]1(CC[C@](C(=O)OC)(N(C(=O)C(F)(F)F)c3cccc(Cl)c3)CC1)[C@@H](C[C@@H](C)COCc1ccc(OC)cc1)C2]

and

### Preparation 14bF methyl (1r,2′R,4R)-4-[(3-chlorophenyl)(trifluoroacetyl)amino]-6′-(methoxymethoxy)-2′-{(2R)-3-[(4-methoxyphenyl)methoxy]-2-methylpropyl}-2′,3′-dihydrospiro[cyclohexane-1,1′-indene]-4-carboxylate

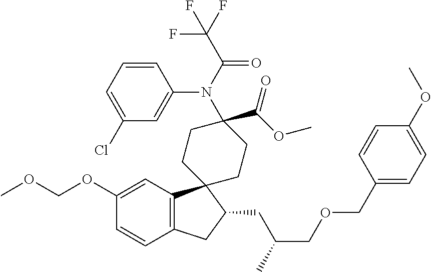 [SMILES: COCOc1ccc2c(c1)[C@]1(CC[C@](C(=O)OC)(N(C(=O)C(F)(F)F)c3cccc(Cl)c3)CC1)[C@H](C[C@@H](C)COCc1ccc(OC)cc1)C2]

Using General procedure 19 and Preparation 14aE as the appropriate indene and toluene instead of EtOAc, a mixture of diastereoisomers was obtained. The diastereoisomers were separated by flash chromatography using heptane and EtOAc as eluents. The diastereoisomer eluting earlier was collected as Preparation 14bF. ¹H NMR (500 MHz, DMSO-d₆) δ ppm: 7.67-7.41 (m, 4H), 7.22 (dm, 2H), 7.05 (d, 1H), 6.89 (dm, 2H), 6.77 (dm, 1H), 6.69 (d, 1H), 5.11 (s, 2H), 4.41/4.36 (d+d, 2H), 3.78 (s, 3H), 3.73 (s, 3H), 3.36 (s, 3H), 3.32/3.10/3.07 (m+dd/dd, 2H), 2.89/2.46 (dd+dd, 2H), 2.29-1.35 (m, 8H), 2.17 (m, 1H), 1.73 (m, 1H), 1.12/0.83 (m+m, 2H), 0.87/0.85 (d/d, 3H). HRMS calculated for C₃₈H₄₃ClF₃NO₇: 717.268; found 735.2976 (M+NH₄).

The diastereoisomer eluting later was collected as Preparation 14aF. ¹H NMR (500 MHz, DMSO-d₆) δ ppm: 7.74-7.40 (m, 4H), 7.16/7.14 (dm/dm, 2H), 7.05 (d, 1H), 6.86/6.85 (dm/dm, 2H), 6.77 (dm, 1H), 6.67/6.66 (d/d, 1H), 5.11 (s, 2H), 4.31/4.28 (s/s, 2H), 3.78 (s, 3H), 3.72 (s, 3H), 3.36 (s, 3H), 3.17-2.99 (m, 2H), 2.90/2.87/2.40 (dd/dd+d, 2H), 2.44-1.18 (m, 8H), 2.20/2.15 (m/m, 1H), 1.65 (m, 1H), 1.03/0.94/0.75/0.65 (m/m+m/m, 2H), 0.77/0.74 (d/d, 3H). HRMS calculated for C₃₈H₄₃ClF₃NO₇: 717.268; found 735.2977 (M+NH₄).

### Preparation 14aG methyl (1r,2′S,4S)-4-[(3-chlorophenyl)(trifluoroacetyl)amino]-2′-[(2R)-3-hydroxy-2-methylpropyl]-6′-(methoxymethoxy)-2′,3′-dihydrospiro[cyclohexane-1,1′-indene]-4-carboxylate

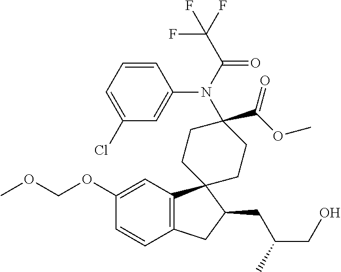 [SMILES: COCOc1ccc2c(c1)[C@]1(CC[C@](C(=O)OC)(N(C(=O)C(F)(F)F)c3cccc(Cl)c3)CC1)[C@@H](C[C@@H](C)CO)C2]

### Preparation 14bG methyl (1r,2′R,4R)-4-[(3-chlorophenyl)(trifluoroacetyl)amino]-2′-[(2R)-3-hydroxy-2-methylpropyl]-6′-(methoxymethoxy)-2′,3′-dihydrospiro[cyclohexane-1,1′-indene]-4-carboxylate

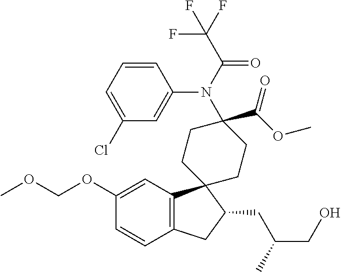 [SMILES: COCOc1ccc2c(c1)[C@]1(CC[C@](C(=O)OC)(N(C(=O)C(F)(F)F)c3cccc(Cl)c3)CC1)[C@H](C[C@@H](C)CO)C2]

Using General procedure 28a and Preparation 14aF as the appropriate PMB derivative, Preparation 14aG was obtained. ¹H NMR (500 MHz, DMSO-d₆) δ ppm: 7.74-7.42 (m, 4H), 7.05 (d, 1H), 6.77/6.76 (dd, 1H), 6.67/6.66 (d, 1H), 5.11 (s, 2H), 4.37/4.34 (br t, 1H), 3.79 (s, 3H), 3.36 (s, 3H), 3.19-2.96 (m, 2H), 2.88/2.40 (dd+dd, 2H), 2.47-1.17 (m, 8H), 2.21/2.16 (m, 1H), 1.45 (m, 1H), 1.04/0.95/0.70/0.59 (m+m, 2H), 0.73/0.70 (d, 3H). HRMS calculated for C₃₀H₃₅ClF₃NO₆: 597.2105; found 615.2434 (M+NH₄).

Using General procedure 28a and Preparation 14bF as the appropriate PMB derivative, Preparation 14bG was obtained. ¹H NMR (500 MHz, DMSO-d₆) δ ppm: 7.70-7.48 (m, 4H), 7.05 (d, 1H), 6.77 (dd, 1H), 6.68 (d, 1H), 5.11 (s, 2H), 4.38/4.36 (t/t, 1H), 3.78 (s, 3H), 3.38/3.07 (m+m, 2H), 3.36 (s, 3H), 2.90/2.43 (dm+d, 2H), 2.22-1.40 (m, 8H), 2.21 (m, 1H), 1.51 (m, 1H), 1.12/0.69 (m+m, 2H), 0.82 (d, 3H). HRMS calculated for C₃₀H₃₅ClF₃NO₆: 597.2105; found 615.2440 (M+NH₄).

### Preparation 14aH methyl (1r,2′S,4S)-4-[(3-chlorophenyl)(trifluoroacetyl)amino]-6′-(methoxymethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

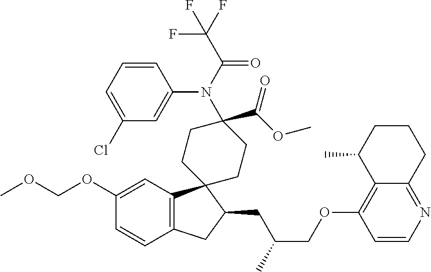 [SMILES: COCOc1ccc2c(c1)[C@]1(CC[C@](C(=O)OC)(N(C(=O)C(F)(F)F)c3cccc(Cl)c3)CC1)[C@@H](C[C@@H](C)COc1ccnc3c1[C@H](C)CCC3)C2]

### Preparation 14bH methyl (1r,2′S,4S)-4-[(3-chlorophenyl)(trifluoroacetyl)amino]-6′-(methoxymethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

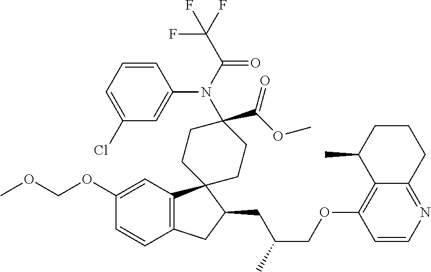 [SMILES: COCOc1ccc2c(c1)[C@]1(CC[C@](C(=O)OC)(N(C(=O)C(F)(F)F)c3cccc(Cl)c3)CC1)[C@@H](C[C@@H](C)COc1ccnc3c1[C@@H](C)CCC3)C2]

Using General procedure 30a and Preparation 14aG as the appropriate indane and Preparation 2a1 as the appropriate alcohol, Preparation 14aH was obtained. ¹H NMR (500 MHz, DMSO-d₆) δ ppm: 8.12/8.10 (d/d, 1H), 7.79-7.42 (m, 4H), 7.04 (d, 1H), 6.78/6.77 (dd/dd, 1H), 6.71/6.68 (d/d, 1H), 6.66 (d, 1H), 5.11 (s, 2H), 3.79 (s, 3H), 3.74 (m, 2H), 3.35 (s, 3H), 2.94/2.44 (m+m, 2H), 2.90 (m, 1H), 2.74/2.63 (m+m, 2H), 2.51-1.20 (m, 8H), 2.30/2.25 (m/m, 1H), 1.89 (m, 1H), 1.77/1.73 (m+m, 2H), 1.60 (m, 2H), 1.23-0.81 (m, 2H), 0.91/0.86 (d/d, 3H), 0.91/0.90 (d/d, 3H).

Using General procedure 30a and Preparation 14aG as the appropriate indane and Preparation 2a2 as the appropriate alcohol, Preparation 14bH was obtained. ¹H NMR (500 MHz, DMSO-d₆) δ ppm: 8.11/8.09 (d/d, 1H), 7.81-7.45 (m, 4H), 7.05 (d, 1H), 6.78/6.77 (dd/dd, 1H), 6.66 (d, 1H), 6.65/6.63 (d/d, 1H), 5.11/5.10 (s/s, 2H), 3.81/3.78/3.68/3.64 (dd+dd/dd+dd, 2H), 3.80 (s, 3H), 3.35/3.34 (s/s, 3H), 2.95/2.48 (m+m, 2H), 2.83/2.77 (m/m, 1H), 2.73/2.62 (m+m, 2H), 2.58-1.18 (m, 8H), 2.34/2.27 (m/m, 1H), 1.90 (m, 1H), 1.78/1.72 (m+m, 2H), 1.60 (m, 2H), 1.02/0.97 (d/d, 3H), 1.00/0.94 (m+m, 2H), 0.92 (d, 3H). HRMS calculated for C₄₀H₄₆N₂O₆F₃Cl: 742.2996; found: 743.3049 (M+H).

### Preparation 14a methyl (1r,2′S,4S)-4-[(3-chlorophenyl)(trifluoroacetyl)amino]-6′-hydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

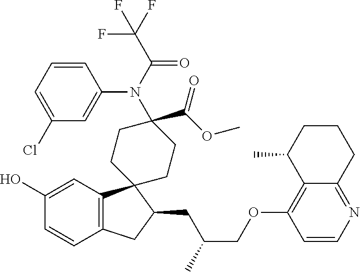 [SMILES: COC(=O)[C@]1(N(C(=O)C(F)(F)F)c2cccc(Cl)c2)CC[C@@]2(CC1)c1cc(O)ccc1C[C@@H]2C[C@@H](C)COc1ccnc2c1[C@H](C)CCC2]

Preparation 14aH (3.30 g, 4.44 mmol) was dissolved in DCM (44 mL). 1.25 M HCl solution in EtOH (10.6 mL, 13.3 mmol) was added and the mixture was stirred at rt overnight. Then it was diluted with water, sat. aq. NaHCO₃ solution and extracted with DCM. The combined organic layers were dried over Na₂SO₄, filtered and the filtrate was concentrated under reduced pressure. The crude product was purified via flash chromatography using MeOH and DCM as eluents to obtain Preparation 14a. ¹H NMR (500 MHz, DMSO-d₆) δ ppm: 9.04/9.03 (s/s, 1H), 8.12/8.10 (d/d, 1H), 7.80-7.40 (m, 4H), 6.90 (d, 1H), 6.71/6.68 (d/d, 1H), 6.49 (dd, 1H), 6.45/6.43 (d/d, 1H), 3.81-3.68 (m, 2H), 3.78 (s, 3H), 2.91 (m, 1H), 2.88/2.37 (m+d, 2H), 2.74/2.63 (m+m, 2H), 2.50-1.35 (m, 8H), 2.25/2.20 (m/m, 1H), 1.87 (m, 1H), 1.77/1.73 (m+m, 2H), 1.60 (m, 2H), 1.24-0.80 (m, 2H), 0.91/0.87 (d/d, 3H), 0.91/0.89 (d/d, 3H). HRMS calculated for C₃₈H₄₂ClF₃N₂O₅: 698.2734; found 699.2800 (M+H).

### Preparation 14b methyl (1r,2′S,4S)-4-[(3-chlorophenyl)(trifluoroacetyl)amino]-6′-hydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

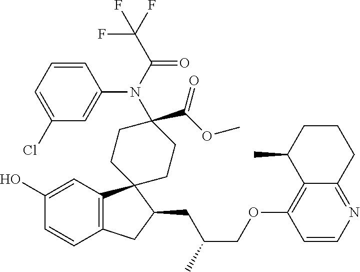 [SMILES: COC(=O)[C@]1(N(C(=O)C(F)(F)F)c2cccc(Cl)c2)CC[C@@]2(CC1)c1cc(O)ccc1C[C@@H]2C[C@@H](C)COc1ccnc2c1[C@@H](C)CCC2]

Preparation 14bH (2.294 g, 33.09 mmol) was dissolved in DCM (330 mL). 1.25 M HCl solution in EtOH (15.4 mL, 19.3 mmol) was added and the mixture was stirred at rt for 1 h. Then it was diluted with water, sat. aq. NaHCO₃ solution and extracted with DCM. The combined organic layers were dried over Na₂SO₄, filtered and the filtrate was concentrated under reduced pressure. The crude product was purified via flash chromatography using MeOH and DCM as eluents to obtain Preparation 14b. ¹H NMR (500 MHz, DMSO-d₆) δ ppm: 9.05/9.04 (s/s, 1H), 8.11/8.09 (d/d, 1H), 7.82-7.42 (m, 4H), 6.91 (d, 1H), 6.66/6.63 (d/d, 1H), 6.49 (dd, 1H), 6.44/6.42 (d/d, 1H), 3.85-3.60 (m, 2H), 3.79 (s, 3H), 2.90/2.42 (m+dd, 2H), 2.85/2.79 (m/m, 1H), 2.56-0.86 (m, 14H), 2.29/2.22 (br/br, 1H), 1.88 (m, 1H), 1.78/1.72 (m+m, 2H), 1.02/0.98 (d/d, 3H), 0.92 (d, 3H). HRMS calculated for C₃₈H₄₂N₂O₅F₃Cl: 698.2734; found: 699.2799 (M+H).

### Preparation 14c methyl (1r,2′R,4R)-4-[(3-chlorophenyl)(trifluoroacetyl)amino]-6′-hydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

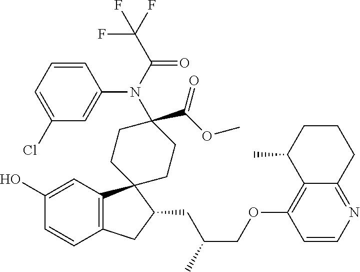 [SMILES: COC(=O)[C@]1(N(C(=O)C(F)(F)F)c2cccc(Cl)c2)CC[C@@]2(CC1)c1cc(O)ccc1C[C@H]2C[C@@H](C)COc1ccnc2c1[C@H](C)CCC2]

Using General procedure 30a and Preparation 14bG (4 g, 6.69 mmol, 1 eq) as the appropriate indane and Preparation 2a1 (1.64 g, 10 mmol, 1.5 eq) as the appropriate alcohol, an intermediate was obtained which was purified by loading onto a DCM-wet SCX cartridge (70 g), washing successively with DCM, MeOH and eluting with 10% NH₃/MeOH in DCM, then further purified by automated flash chromatography (CombiFlash Rf, 40 g RediSep™ silica cartridge) eluting with a gradient of 0-20% MeOH in EtOAc to obtain Preparation 14c as a white solid (2.48 g, 3.55 mmol, 53%). LRMS calculated for C₃₈H₄₂ClF₃N₂O₅: 698; found: 699 (M+H). ¹H NMR (400 MHz, DMSO-d₆) δ ppm: 9.09 (s, 1H), 8.26/8.21 (d, J=5.6 Hz, 1H), 7.62-6.83 (m, 6H), 6.53-6.47 (m, 2H), 4.11-3.98 (m, 1H), 3.87-3.70 (m, 4H), 3.09-2.95 (m, 1H), 2.94-2.73 (m, 2H), 2.73-2.57 (m, 1H), 2.49-2.41 (m, 1H), 2.36-1.36 (m, 15H), 1.14-1.08 (m, 3H), 1.06-0.83 (m, 4H).

### Preparation 15a

### Preparation 15aA methyl (1r,2′S,4S)-5′-chloro-4-[(3-chlorophenyl)(trifluoroacetyl)amino]-2′-[(2R)-3-hydroxy-2-methylpropyl]-6′-(methoxymethoxy)-2′,3′-dihydrospiro[cyclohexane-1,1′-indene]-4-carboxylate

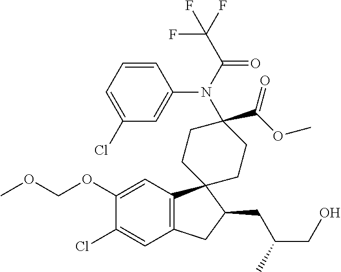 [SMILES: COCOc1cc2c(cc1Cl)C[C@H](C[C@@H](C)CO)[C@]21CC[C@@](C(=O)OC)(N(C(=O)C(F)(F)F)c2cccc(Cl)c2)CC1]

Preparation 14aG (5.0 g, 8.36 mmol) was dissolved in MeCN (100 mL). 1,3-Dichloro-5,5-dimethyl-imidazolidine-2,4-dione (873 mg, 4.43 mmol) was added and the mixture was stirred at rt for 2 days in the dark. Then it was diluted with sat. aq. NaHCO₃ solution and extracted with EtOAc. The combined organic layers were dried over MgSO₄, filtered and the filtrate was concentrated under reduced pressure. The crude product was purified via prep RP-HPLC using 25 mM aq. NH₄HCO₃ solution and MeCN as eluents to obtain Preparation 15aA. ¹H NMR (500 MHz, DMSO-d₆) δ ppm: 7.75-7.43 (m, 4H), 7.22 (s, 1H), 6.90/6.88 (s, 1H), 5.26-5.19 (d+d, 2H), 4.38/4.35 (t, 1H), 3.79/3.78 (s, 3H), 3.42/3.41 (s, 3H), 3.19-2.96 (m, 2H), 2.90/2.41 (dd+dd, 2H), 2.49-0.53 (m, 10H), 2.24/2.18 (m, 1H), 1.44 (m, 1H), 0.73/0.70 (d, 3H). HRMS calculated for C₃₀H₃₄Cl₂F₃NO₆: 631.1715; found 649.2039 (M+NH₄).

### Preparation 15aB methyl (1r,2′S,4S)-5′-chloro-4-[(3-chlorophenyl)(trifluoroacetyl)amino]-6′-(methoxymethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

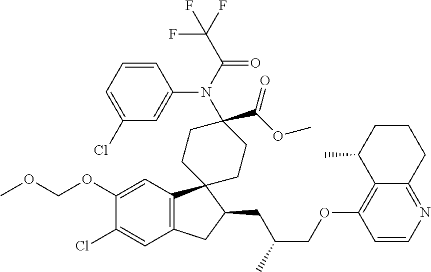 [SMILES: COCOc1cc2c(cc1Cl)C[C@H](C[C@@H](C)COc1ccnc3c1[C@H](C)CCC3)[C@]21CC[C@@](C(=O)OC)(N(C(=O)C(F)(F)F)c2cccc(Cl)c2)CC1]

Using General procedure 30a and Preparation 15aA as the appropriate indane and Preparation 2a1 as the appropriate alcohol, Preparation 15aB was obtained. ¹H NMR (500 MHz, DMSO-d₆) δ ppm: 8.12/8.10 (d/d, 1H), 7.81-7.42 (m, 4H), 7.20 (s, 1H), 6.89/6.88 (s/s, 1H), 6.70/6.68 (d/d, 1H), 5.22 (m, 2H), 3.82-3.64 (m, 2H), 3.79 (s, 3H), 3.40 (s, 3H), 2.96/2.45 (m+d, 2H), 2.89 (m, 1H), 2.74/2.64 (dm+m, 2H), 2.54-0.78 (m, 14H), 2.33/2.27 (m/m, 1H), 1.87 (m, 1H), 0.89 (d, 3H), 0.86/0.81 (d/d, 3H). HRMS calculated for C₄₀H₄₅Cl₂F₃N₂O₆: 776.2607; found 777.2665 (M+H).

### Preparation 15a methyl (1r,2′S,4S)-5′-chloro-4-[(3-chlorophenyl)(trifluoroacetyl)amino]-6′-hydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

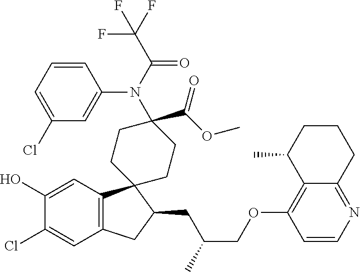 [SMILES: COC(=O)[C@]1(N(C(=O)C(F)(F)F)c2cccc(Cl)c2)CC[C@@]2(CC1)c1cc(O)c(Cl)cc1C[C@@H]2C[C@@H](C)COc1ccnc2c1[C@H](C)CCC2]

Preparation 15aB (2.12 g, 2.73 mmol) was dissolved in DCM (27 mL). 1.25 M HCl solution in EtOH (6.5 mL, 8.18 mmol) was added and the mixture was stirred at rt overnight. Then it was diluted with water and sat. aq. NaHCO₃ solution. It was extracted with DCM. The combined organic layers were dried over MgSO₄, filtered and the filtrate was concentrated under reduced pressure. The crude product was purified via flash chromatography using heptane and EtOAc as eluents to obtain Preparation 15a. ¹H NMR (500 MHz, DMSO-d₆) δ ppm: 9.80 (br s, 1H), 8.12/8.10 (d/d, 1H), 7.84-7.41 (m, 4H), 7.06 (s, 1H), 6.71/6.68 (d/d, 1H), 6.66/6.64 (s/s, 1H), 3.83-3.60 (m, 2H), 3.78 (s, 3H), 2.89 (m, 1H), 2.89/2.39 (m+d, 2H), 2.74/2.64 (dm+m, 2H), 2.50-0.76 (m, 14H), 2.28/2.22 (m/m, 1H), 1.86 (m, 1H), 0.90/0.88 (d/d, 3H), 0.87/0.82 (d/d, 3H). HRMS calculated for C₃₈H₄₁Cl₂F₃N₂O₅: 732.2344; found 733.2423 (M+H).

### Preparation 16a methyl (1r,2′S,4S)-4-[(3-chlorophenyl)(trifluoroacetyl)amino]-2′-[(2R)-2-methyl-3-{[(5R)-5-methyl-5,6,7,8-tetrahydroquinolin-4-yl]oxy}propyl]-6′-[(trifluoromethanesulfonyl)oxy]-2′,3′-dihydrospiro[cyclohexane-1,1′-indene]-4-carboxylate

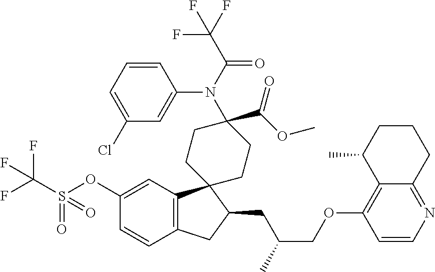 [SMILES: COC(=O)[C@]1(N(C(=O)C(F)(F)F)c2cccc(Cl)c2)CC[C@@]2(CC1)c1cc(OS(=O)(=O)C(F)(F)F)ccc1C[C@@H]2C[C@@H](C)COc1ccnc2c1[C@H](C)CCC2]

Preparation 14a (1.15 g, 1.64 mmol) was dissolved in DCM (16 mL). Pyridine (265 μL, 3.28 mmol) was added and the mixture was cooled to 0° C. 1 M Tf₂O solution in DCM (1.97 mL, 1.97 mmol) was added at 0° C., then it was allowed to warm to rt and stirred for 30 min. Then it was cooled to 0° C., the pH was set to 7 with 0.1 M aq. HCl solution and the layers were separated. The aq. layer was extracted with DCM. The combined organic layers were dried over MgSO₄, filtered and the filtrate was concentrated under reduced pressure. The crude product was purified via flash chromatography using EtOAc and MeOH as eluents to obtain Preparation 16a. ¹H NMR (500 MHz, DMSO-d₆) δ ppm: 8.34/8.33 (d/d, 1H), 7.82-7.44 (m, 4H), 7.34 (d, 1H), 7.23 (dd, 1H), 7.10/7.09 (d/d, 1H), 7.03/7.01 (d/d, 1H), 3.96-3.80 (m, 2H), 3.80/3.79 (s/s, 3H), 3.08/2.58 (m+d, 2H), 2.82/2.73 (m+m, 2H), 2.55-1.19 (m, 8H), 2.41/2.35 (br/br, 1H), 1.93 (m, 1H), 1.76 (m, 2H), 1.69-1.54 (m, 1H), 1.69-1.54 (m, 2H), 1.21-0.82 (m, 2H), 0.92/0.91 (d/d, 3H), 0.85/0.79 (d/d, 3H). HRMS calculated for C₃₉H₄₁ClF₆N₂O₇S: 830.2227; found 831.2292 (M+H).

### Preparation 16b methyl (1r,2′R,4R)-4-[(3-chlorophenyl)(trifluoroacetyl)amino]-2′-[(2R)-2-methyl-3-{[(5R)-5-methyl-5,6,7,8-tetrahydroquinolin-4-yl]oxy}propyl]-6′-[(trifluoromethanesulfonyl)oxy]-2′,3′-dihydrospiro[cyclohexane-1,1′-indene]-4-carboxylate

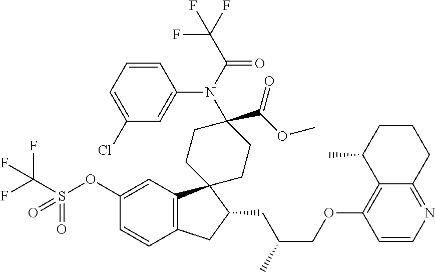 [SMILES: COC(=O)[C@]1(N(C(=O)C(F)(F)F)c2cccc(Cl)c2)CC[C@@]2(CC1)c1cc(OS(=O)(=O)C(F)(F)F)ccc1C[C@H]2C[C@@H](C)COc1ccnc2c1[C@H](C)CCC2]

To a solution of Preparation 14c (1.21 g, 1.73 mmol, 1 eq) in DCM (15 mL), cooled to 0° C., was added pyridine (279 μL, 3.46 mmol, 2 eq) followed by Tf₂O (341 μL, 2.08 mmol, 1.2 eq) and the mixture was stirred at rt for 2 h. The mixture was partitioned between DCM and 0.1 M aq. HCl solution, and the organic phase was washed with sat. aq. NaHCO₃ solution, brine, dried (MgSO₄) and concentrated in vacuo. Purification by automated flash chromatography (CombiFlash Rf, 24 g RediSep™ silica cartridge) eluting with a gradient of 0-4% MeOH in EtOAc afforded Preparation 16b as an off-white solid (731 mg, 0.88 mmol, 51%). LRMS calculated for C₃₉H₄₁ClF₆N₂O₇S: 830; found: 831 (M+H).

### Preparation 17a and Preparation 17b

### Preparation 17aA methyl (1s,4s)-6′-acetyl-2′-bromo-4-(3-chloroanilino)spiro[cyclohexane-1,1′-indene]-4-carboxylate

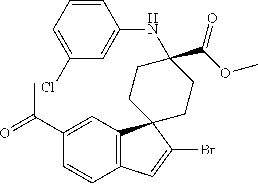 [SMILES: COC(=O)[C@]1(Nc2cccc(Cl)c2)CC[C@]2(CC1)C(Br)=Cc1ccc(C(C)=O)cc12]

Using General procedure 33a and Preparation 14aA as the appropriate ester, an intermediate was obtained which was treated as described in General procedure 17a to obtain Preparation 17aA. ¹H NMR (500 MHz, DMSO-d₆) δ ppm: 8.27 (br s, 1H), 7.99 (dd, 1H), 7.51 (d, 1H), 7.18 (s, 1H), 7.10 (t, 1H), 6.61 (m, 1H), 6.60 (dm, 1H), 6.53 (s, 1H), 6.47 (dm, 1H), 3.72 (s, 3H), 2.61 (s, 3H), 2.44/2.31 (td+br d, 4H), 2.22/1.03 (td+br d, 4H). HRMS calculated for C₂₄H₂₃BrClNO₃: 487.055; found 488.0618 (M+H).

### Preparation 17aB methyl (1r,4R)-6′-acetyl-4-(3-chloroanilino)-2′-{(2R)-3-[(4-methoxyphenyl)methoxy]-2-methylpropyl}spiro[cyclohexane-1,1′-indene]-4-carboxylate

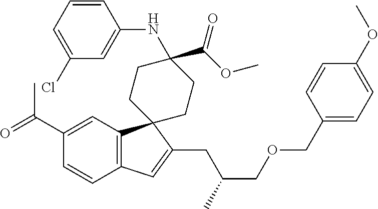 [SMILES: COc1ccc(COC[C@H](C)CC2=Cc3ccc(C(C)=O)cc3[C@]23CC[C@@](Nc2cccc(Cl)c2)(C(=O)OC)CC3)cc1]

Using General procedure 27b and Preparation 17aA as the appropriate 2-bromo-indene derivative and Preparation 3d as the appropriate Zn reagent, Preparation 17aB was obtained. ¹H NMR (400 MHz, DMSO-d₆) δ ppm: 8.24 (s, 1H), 7.91 (dd, 1H), 7.38 (d, 1H), 7.21 (d, 2H), 7.09 (t, 1H), 6.85 (d, 2H), 6.66 (t, 1H), 6.60 (dd, 1H), 6.56 (s, 1H), 6.48 (dd, 1H), 6.45 (s, 1H), 4.38 (m, 2H), 3.72 (s, 3H), 3.72 (s, 3H), 3.30 (m, 2H), 2.58 (s, 3H), 2.43/2.24 (m+m, 4H), 2.41/2.03 (m+m, 2H), 2.18 (m, 1H), 2.12/0.88 (m+m, 4H), 0.93 (d, 3H). HRMS calculated for C₃₆H40ClNO₅: 601.2595; found 602.26625 (M+H).

### Preparation 17aC methyl (1r,4R)-6′-acetyl-4-(3-chloroanilino)-2′-[(2R)-3-hydroxy-2-methylpropyl]spiro[cyclohexane-1,1′-indene]-4-carboxylate

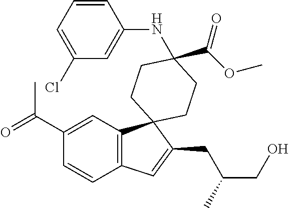

Using General procedure 28a and Preparation 17aB as the appropriate PMB derivative, Preparation 17aC was obtained. ¹H NMR (500 MHz, DMSO-d₆) δ ppm: 8.26 (br s, 1H), 7.93 (dd, 1H), 7.42 (d, 1H), 7.11 (t, 1H), 6.65 (t, 1H), 6.61 (dm, 1H), 6.60 (s, 1H), 6.48 (dm, 1H), 6.46 (s, 1H), 4.59 (t, 1H), 3.73 (s, 3H), 3.34 (m, 2H), 2.60 (s, 3H), 2.50-0.84 (m, 8H), 2.43/1.97 (m+m, 2H), 2.00 (m, 1H), 0.91 (d, 3H). HRMS calculated for C₂₈H₃₂ClNO₄: 481.202; found 482.2088 (M+H).

### Preparation 17a methyl (1r,2′S,4S)-6′-acetyl-4-(3-chloroanilino)-2′-[(2R)-3-hydroxy-2-methylpropyl]-2′,3′-dihydrospiro[cyclohexane-1,1′-indene]-4-carboxylate

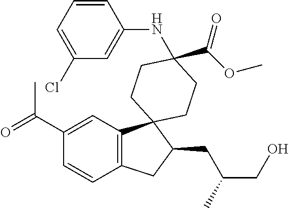 [SMILES: COC(=O)[C@]1(Nc2cccc(Cl)c2)CC[C@@]2(CC1)c1cc(C(C)=O)ccc1C[C@@H]2C[C@@H](C)CO]

and

### Preparation 17b methyl (1r,2′R,4R)-6′-acetyl-4-(3-chloroanilino)-2′-[(2R)-3-hydroxy-2-methylpropyl]-2′,3′-dihydrospiro[cyclohexane-1,1′-indene]-4-carboxylate

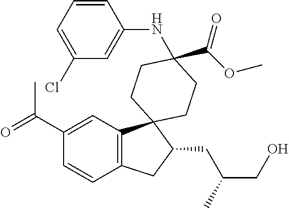 [SMILES: COC(=O)[C@]1(Nc2cccc(Cl)c2)CC[C@@]2(CC1)c1cc(C(C)=O)ccc1C[C@H]2C[C@@H](C)CO]

Using General procedure 19 and Preparation 17aC as the appropriate indene and toluene instead of EtOAc, a mixture of diastereoisomers was obtained. The diastereoisomers were separated by chiral chromatography. Column: AD, 100×500 mm, 20 μm. Eluents: 50:50 iPrOH/heptane. The diastereoisomer eluting earlier was collected as Preparation 17b. ¹H NMR (500 MHz, DMSO-d₆) δ ppm: 7.91 (br s, 1H), 7.81 (dd, 1H), 7.35 (d, 1H), 7.07 (t, 1H), 6.59 (t, 1H), 6.57 (dm, 1H), 6.45 (dm, 1H), 6.32 (s, 1H), 4.41 (t, 1H), 3.66 (s, 3H), 3.41/3.21 (m+m, 2H), 3.05/2.61 (dd+dd, 2H), 2.56 (s, 3H), 2.52-1.30 (m, 8H), 2.16 (m, 1H), 1.61 (m, 1H), 1.47/1.00 (m+td, 2H), 0.90 (d, 3H). HRMS calculated for C₂₈H₃₄ClNO₄: 483.2176; found 484.2245 (M+H).

The diastereoisomer eluting later was collected as Preparation 17a. ¹H NMR (500 MHz, DMSO-d₆) δ ppm: 7.90 (br s, 1H), 7.81 (dd, 1H), 7.35 (d, 1H), 7.07 (t, 1H), 6.60 (t, 1H), 6.57 (dm, 1H), 6.45 (dm, 1H), 6.34 (s, 1H), 4.46 (t, 1H), 3.66 (s, 3H), 3.24/3.20 (m+m, 2H), 3.02/2.57 (dd+dd, 2H), 2.56 (s, 3H), 2.52-1.36 (m, 8H), 2.16 (m, 1H), 1.57 (m, 1H), 1.32/1.07 (m+m, 2H), 0.86 (d, 3H). HRMS calculated for C₂₈H₃₄ClNO₄: 483.2176; found 484.2250 (M+H).

### Preparation 18a

### Preparation 18aA 6-methoxy-1H-indene

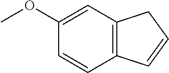

5-methoxy-2,3-dihydro-1H-inden-1-one (50.7 g, 313 mmol) was dissolved in MeOH (500 mL) and cooled to 0° C. NaBH₄ (24.8 g, 655 mmol) was added portionwise and then the mixture was allowed to warm to rt and stirred for 1 h. Then it was concentrated under reduced pressure. The residue was diluted with water and extracted with EtOAc. The combined organic layers were dried over Na₂SO₄, filtered and the filtrate was concentrated under reduced pressure. The residue was taken up in THE (300 mL). PTSA (3.0 g, 15.6 mmol) was added and the mixture was stirred at 75° C. overnight. Then it was washed with sat. aq. NaHCO₃ solution and brine, dried over Na₂SO₄, filtered and the filtrate was concentrated under reduced pressure. The crude product was purified via flash chromatography using heptane and EtOAc as eluents to obtain Preparation 18aA. ¹H NMR (500 MHz, DMSO-d₆) δ ppm: 7.30 (d, 1H), 7.11 (d, 1H), 6.84 (dt, 1H), 6.83 (dd, 1H), 6.44 (dt, 1H), 3.75 (s, 3H), 3.36 (t, 2H). HRMS calculated for C₁₀H₁₀O: 146.0732; found 146.07341 (M+).

### Preparation 18aB 2-bromo-6-methoxy-1H-indene

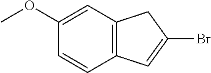

Preparation 18aA (12.0 g, 82.4 mmol) was dissolved in DMSO (100 mL) and cooled to 0° C. Water (2.8 mL) and then NBS (15.0 g, 84.4 mmol) were added portionwise. Then it was allowed to warm to rt and stirred for 30 min. Then it was poured onto ice and the precipitate was filtered. The precipitate was taken up in EtOAc, dried over Na₂SO₄, filtered and the filtrate was concentrated under reduced pressure. The residue was taken up in toluene (800 mL). PTSA (1.7 g, 8.9 mmol) was added and the mixture was stirred at 80° C. overnight. Then it was cooled to rt, washed with sat. aq. NaHCO₃ solution and brine. The aq. layer was extracted with toluene. The combined organic layers were dried over Na₂SO₄, filtered and the filtrate was concentrated under reduced pressure. The crude product was purified via flash chromatography using heptane and EtOAc as eluents to obtain Preparation 18aB. ¹H NMR (500 MHz, DMSO-d₆) δ ppm: 7.25 (d, 1H), 7.04 (m, 1H), 7.02 (td, 1H), 6.82 (dd, 1H), 3.75 (s, 3H), 3.64 (s, 2H). HRMS calculated for C₁₀H₉BrO: 223.9837; found 223.98418 (M+).

### Preparation 18aC 2″-bromo-6″-methoxydispiro[[1,3]dioxolane-2,1′-cyclohexane-4′,1″-indene]

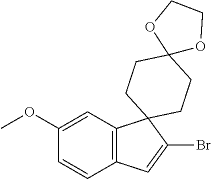 [SMILES: COc1ccc2c(c1)C1(CCC3(CC1)OCCO3)C(Br)=C2]

and

### Preparation 18aD 2″-bromo-5″-methoxydispiro[[1,3]dioxolane-2,1′-cyclohexane-4′,1″-indene]

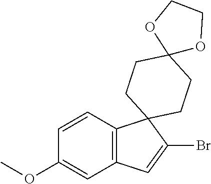 [SMILES: COc1ccc2c(c1)C=C(Br)C21CCC2(CC1)OCCO2]

Using General procedure 8a and Preparation 18aB as the appropriate indene, a mixture of regioisomers was obtained. The regioisomers were separated via flash chromatography using heptane and EtOAc as eluents. The regioisomer eluting earlier was collected as Preparation 18aD. ¹H NMR (500 MHz, DMSO-d₆) δ ppm: 7.56 (d, 1H), 6.99 (s, 1H), 6.95 (d, 1H), 6.75 (dd, 1H), 3.95 (m, 4H), 3.75 (s, 3H), 2.15-1.07 (m, 8H). HRMS calculated for C₁₇H₁₉BrO₃: 350.0518; found 351.0593 (M+H).

The regioisomer eluting later was collected as Preparation 18aC. ¹H NMR (500 MHz, DMSO-d₆) δ ppm: 7.27 (d, 1H), 7.15 (d, 1H), 6.95 (s, 1H), 6.88 (dd, 1H), 3.95 (m, 4H), 3.77 (s, 3H), 2.15-1.07 (m, 8H). HRMS calculated for C₁₇H₁₉BrO₃: 350.0518; found 351.0596 (M+H).

### Preparation 18aE 2′-bromo-6′-methoxyspiro[cyclohexane-1,1′-inden]-4-one

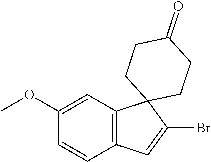 [SMILES: COc1ccc2c(c1)C1(CCC(=O)CC1)C(Br)=C2]

Using General procedure 9 and Preparation 18aC as the appropriate ketal, Preparation 18aE was obtained. ¹H NMR (400 MHz, DMSO-d₆) δ ppm: 7.44 (d, 1H), 7.29 (d, 1H), 7.01 (s, 1H), 6.88 (dd, 1H), 3.79 (s, 3H), 2.91/2.52 (m, 4H), 2.17/1.66 (m, 4H). LRMS calculated for C₁₅H₁₅BrO₂: 306.0; found 306.0 (M+).

### Preparation 18aF (1s,4s)-2′-bromo-4-(3-chloroanilino)-6′-methoxyspiro[cyclohexane-1,1′-indene]-4-carbonitrile

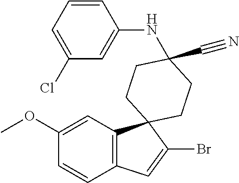 [SMILES: COc1ccc2c(c1)[C@]1(CC[C@](C#N)(Nc3cccc(Cl)c3)CC1)C(Br)=C2]

Using General procedure 11 and Preparation 18aE as the appropriate ketone and 3-chloroaniline as the appropriate aniline, a mixture of diastereoisomers was obtained. The diastereoisomers were separated via flash chromatography using heptane and EtOAc as eluents. The diastereoisomer eluting earlier was collected as Preparation 18aF. ¹H NMR (500 MHz, DMSO-d₆) δ ppm: 7.28 (d, 1H), 7.24 (t, 1H), 7.22 (d, 1H), 6.97 (s, 1H), 6.92 (t, 1H), 6.89 (m, 2H), 6.79 (dm, 1H), 6.59 (s, 1H), 3.80 (s, 3H), 2.55/2.47 (m+m, 4H), 2.06/1.35 (m+m, 4H). HRMS calculated for C₂₂H₂₀BrClN₂O: 442.0447; found 443.0526 (M+H).

### Preparation 18aG (1s,4s)-2′-bromo-4-(3-chloroanilino)-6′-methoxyspiro[cyclohexane-1,1′-indene]-4-carboxamide

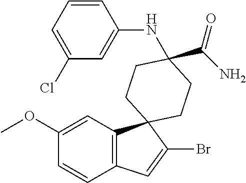 [SMILES: COc1ccc2c(c1)[C@]1(CC[C@@](Nc3cccc(Cl)c3)(C(N)=O)CC1)C(Br)=C2]

Using General procedure 12b and Preparation 18aF as the appropriate nitrile, Preparation 18aG was obtained. ¹H NMR (500 MHz, DMSO-d₆) δ ppm: 7.37 (d, 1H), 7.34/7.24 (d+d, 2H), 7.26 (d, 1H), 7.12 (t, 1H), 6.93 (s, 1H), 6.88 (dd, 1H), 6.69 (t, 1H), 6.62 (dm, 1H), 6.60 (dm, 1H), 6.23 (s, 1H), 3.78 (s, 3H), 2.47/2.09 (m+m, 4H), 2.09/0.95 (m+m, 4H). HRMS calculated for C₂₂H₂₂BrClN₂O₂: 460.0553; found 461.0639 (M+H).

### Preparation 18aH (1s,4s)-2′-bromo-4-(3-chloroanilino)-6′-methoxyspiro[cyclohexane-1,1′-indene]-4-carboxylic acid

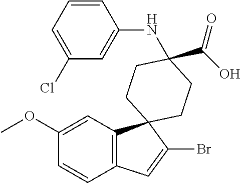 [SMILES: COc1ccc2c(c1)[C@]1(CC[C@@](Nc3cccc(Cl)c3)(C(=O)O)CC1)C(Br)=C2]

Using General procedure 13 and Preparation 18aG as the appropriate amide, Preparation 18aH was obtained. ¹H NMR (500 MHz, DMSO-d₆) δ ppm: 7.34 (d, 1H), 7.26 (d, 1H), 7.03 (t, 1H), 6.92 (s, 1H), 6.88 (dd, 1H), 6.60 (t, 1H), 6.54 (dm, 1H), 6.50 (dm, 1H), 6.26 (br s, 1H), 3.77 (s, 3H), 2.40/2.17 (m+m, 4H), 2.14/0.95 (m+m, 4H). HRMS calculated for C₂₂H₂₁BrClNO₃: 461.0393; found 462.0465 (M+H).

### Preparation 18aI methyl (1s,4s)-2′-bromo-4-(3-chloroanilino)-6′-methoxyspiro[cyclohexane-1,1′-indene]-4-carboxylate

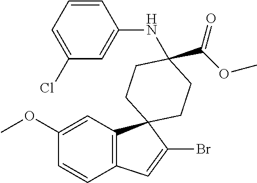

Using General procedure 17a and Preparation 18aH as the appropriate amino acid, Preparation 18aI was obtained. ¹H NMR (500 MHz, DMSO-d₆) δ ppm: 7.28 (d, 1H), 7.26 (s, 1H), 7.09 (t, 1H), 6.95 (s, 1H), 6.90 (dd, 1H), 6.60 (t, 1H), 6.59 (dm, 1H), 6.49 (s, 1H), 6.46 (dm, 1H), 3.79 (s, 3H), 3.69 (s, 3H), 2.40/2.24 (m+m, 4H), 2.18/1.00 (m+m, 4H). HRMS calculated for C₂₃H₂₃BrClNO₃: 475.055; found 476.0620 (M+H).

### Preparation 18aJ methyl (1r,4r)-4-(3-chloroanilino)-6′-methoxy-2′-(3-phenoxyphenyl)spiro[cyclohexane-1,1′-indene]-4-carboxylate

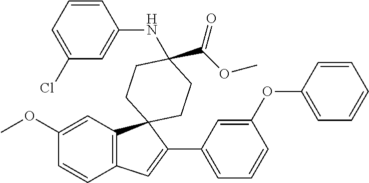 [SMILES: COc1ccc2c(c1)[C@]1(CC[C@@](Nc3cccc(Cl)c3)(C(=O)OC)CC1)C(c1cccc(Oc3ccccc3)c1)=C2]

Using General procedure 18 and Preparation 18aI as the appropriate 2-bromo-indene and (3-phenoxyphenyl)boronic acid as the appropriate boronic acid, Preparation 18aJ was obtained. ¹H NMR (500 MHz, DMSO-d₆) δ ppm: 7.54 (dm, 1H), 7.45 (t, 1H), 7.37 (t, 1H), 7.36 (m, 2H), 7.35 (d, 1H), 7.32 (d, 1H), 7.20 (s, 1H), 7.12 (m, 1H), 7.04 (t, 1H), 7.02 (m, 2H), 6.92 (dd, 1H), 6.89 (dm, 1H), 6.66 (t, 1H), 6.66 (s, 1H), 6.59 (dm, 1H), 6.52 (dm, 1H), 3.81 (s, 3H), 3.70 (s, 3H), 2.51/1.07 (m+m, 4H), 2.43/2.28 (m+m, 4H). HRMS calculated for C₃₅H₃₂ClNO₄: 565.202; found 566.2099 (M+H).

### Preparation 18aK methyl (1r,4r)-4-(3-chloroanilino)-6′-methoxy-2′-(3-phenoxyphenyl)-2′,3′-dihydrospiro[cyclohexane-1,1′-indene]-4-carboxylate, enantiomer 1

and

### Preparation 18aL methyl (1r,4r)-4-(3-chloroanilino)-6′-methoxy-2′-(3-phenoxyphenyl)-2′,3′-dihydrospiro[cyclohexane-1,1′-indene]-4-carboxylate, enantiomer 2

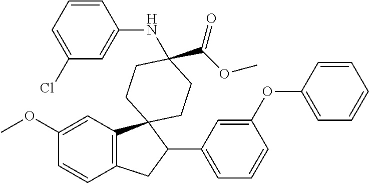

Using General procedure 19 and Preparation 18aJ as the appropriate indene and AcOH instead of EtOAc, a racemate was obtained. The enantiomers were separated by chiral chromatography. Column: AD, 100×500 mm, 20 μm. Eluents: 50:50 iPrOH/heptane. The enantiomer eluting earlier was collected as Preparation 18aK. ¹H NMR (500 MHz, DMSO-d₆) δ ppm: 7.24 (t, 1H), 7.23 (m, 2H), 7.15 (d, 1H), 7.04 (m, 1H), 7.04 (t, 1H), 6.88 (dm, 1H), 6.87 (m, 2H), 6.81 (dm, 1H), 6.81 (d, 1H), 6.76 (dd, 1H), 6.72 (br s, 1H), 6.56 (dm, 1H), 6.48 (t, 1H), 6.37 (dm, 1H), 6.13 (s, 1H), 3.74 (s, 3H), 3.63 (s, 3H), 3.40 (dd, 1H), 3.25/2.92 (dd+dd, 2H), 2.43-1.25 (m, 8H). HRMS calculated for C₃₅H₃₄ClNO₄: 567.2177; found 568.2242 (M+H).

The enantiomer eluting later was collected as Preparation 18aL. LRMS calculated for C₃₅H₃₄ClNO₄: 567.2; found 568.3 (M+H).

### Preparation 18a methyl (1r,4r)-4-(3-chloroanilino)-6′-hydroxy-2′-(3-phenoxyphenyl)-2′,3′-dihydrospiro[cyclohexane-1,1′-indene]-4-carboxylate, enantiomer 1

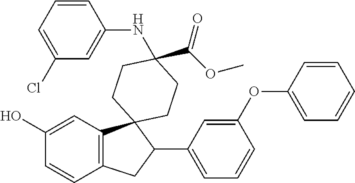

Preparation 18aK (97 mg, 0.17 mmol) was dissolved in DCM (2 mL). 1 M BBr₃ solution in DCM (340 μL, 0.34 mmol) was added and the mixture was stirred at rt for 30 min. Then it was diluted with water and extracted with DCM. The combined organic layers were dried over Na₂SO₄, filtered and the filtrate was concentrated under reduced pressure. The residue was taken up in DCM (1 mL) and MeOH (1 mL). 2 M TMS-CHNN solution in Et₂O (170 μL, 0.34 mmol) was added and the mixture was stirred at rt for 30 min. Then it was concentrated under reduced pressure and purified via flash chromatography using heptane and EtOAc as eluents to obtain Preparation 18a. ¹H NMR (500 MHz, DMSO-d₆) δ ppm: 9.17 (s, 1H), 7.23 (m, 3H), 7.04 (t, 1H), 7.03 (m, 1H), 7.01 (d, 1H), 6.87 (dm, 1H), 6.86 (m, 2H), 6.80 (dm, 1H), 6.74 (br s, 1H), 6.71 (d, 1H), 6.57 (dd, 1H), 6.56 (dm, 1H), 6.48 (t, 1H), 6.37 (dm, 1H), 6.12 (s, 1H), 3.63 (s, 3H), 3.37 (dd, 1H), 3.20/2.87 (dd+dd, 2H), 2.39-1.25 (m, 8H). HRMS calculated for C₃₄H₃₂ClNO₄: 553.202; found 554.2091 (M+H).

### Preparation 19a and Preparation 19b

### Preparation 19aA 5-(benzyloxy)-2,3-dihydro-1H-inden-1-one

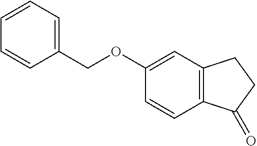 [SMILES: O=C1CCc2cc(OCc3ccccc3)ccc21]

5-hydroxyindan-1-one (444 mg, 3.0 mmol) was dissolved in MeCN (6 mL). K₂CO₃ (912 mg, 6.6 mmol) and bromomethylbenzene (392 μL, 3.3 mmol) was added and the mixture was stirred at rt for 5.5 h. Then it was diluted with water and extracted with EtOAc. The combined organic layers were dried over Na₂SO₄, filtered and the filtrate was concentrated under reduced pressure. The crude product was purified via flash chromatography using heptane and EtOAc as eluents to obtain Preparation 19aA. ¹H NMR (500 MHz, DMSO-d₆) δ ppm: 7.56 (d, 1H), 7.49-7.32 (m, 5H), 7.19 (d, 1H), 7.03 (dd, 1H), 5.22 (s, 2H), 3.04 (m, 2H), 2.58 (m, 2H). HRMS calculated for C₁₆H₁₄O₂: 238.0994; found 239.1071 (M+H).

### Preparation 19aB 5-(benzyloxy)-2-bromo-2,3-dihydro-1H-inden-1-one

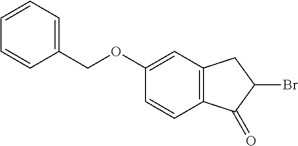 [SMILES: O=C1c2ccc(OCc3ccccc3)cc2CC1Br]

Preparation 19aA (119 mg, 0.5 mmol) was dissolved in CHCl₃ (2 mL) and EtOAc (2 mL).

CuBr₂ (223 mg, 1.0 mmol) was added portionwise and the mixture was stirred at 60° C. for 8 h. Then it was filtered through a pad of Celite, washed with EtOAc and the filtrate was concentrated under reduced pressure. The crude product was purified via flash chromatography using heptane and EtOAc as eluents to obtain Preparation 19aB. ¹H NMR (500 MHz, DMSO-d₆) δ ppm: 7.69 (d, 1H), 7.47 (d, 2H), 7.41 (t, 2H), 7.36 (t, 1H), 7.20 (d, 1H), 7.12 (dd, 1H), 5.25 (s, 2H), 4.97 (dd, 1H), 3.84/3.27 (dd+dd, 2H). HRMS calculated for C₁₆H₁₃BrO₂: 316.0099; found 317.0182 (M+H).

### Preparation 19aC 5-(benzyloxy)-2-bromo-2,3-dihydro-1H-inden-1-ol

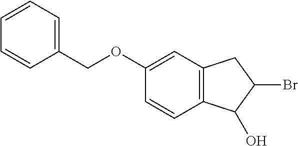 [SMILES: OC1c2ccc(OCc3ccccc3)cc2CC1Br]

Using General procedure 6 and Preparation 19aB as the appropriate bromo-indan-1-one and MeOH as solvent, Preparation 19C was obtained. ¹H NMR (500 MHz, DMSO-d₆) δ ppm: 7.44 (d, 2H), 7.39 (t, 2H), 7.32 (t, 1H), 7.21 (d, 1H), 6.91 (d, 1H), 6.87 (dd, 1H), 5.64 (br s, 1H), 5.10/5.07 (d+d, 2H), 4.84 (m, 1H), 4.83 (m, 1H), 3.36/3.15 (dd+dd, 2H). HRMS calculated for C₁₆H₁₅BrO₂: 318.0255; found 318.02499 (M+).

### Preparation 19aD 6-(benzyloxy)-2-bromo-1H-indene

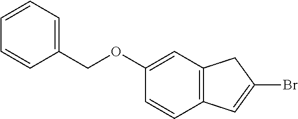 [SMILES: BrC1=Cc2ccc(OCc3ccccc3)cc2C1]

Using General procedure 7 and Preparation 19aC as the appropriate indane, Preparation 19aD was obtained. ¹H NMR (500 MHz, DMSO-d₆) δ ppm: 7.44 (d, 2H), 7.39 (t, 2H), 7.33 (t, 1H), 7.25 (d, 1H), 7.13 (d, 1H), 7.02 (dd, 1H), 6.90 (dd, 1H), 5.09 (s, 2H), 3.65 (dd, 2H). HRMS calculated for C₁₆H₁₃BrO: 300.0150; found 300.01360 (M+).

### Preparation 19aE 6″-(benzyloxy)-2″-bromodispiro[[1,3]dioxolane-2,1′-cyclohexane-4′,1″-indene]

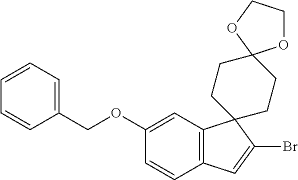 [SMILES: BrC1=Cc2ccc(OCc3ccccc3)cc2C12CCC1(CC2)OCCO1]

Using General procedure 8b and Preparation 19aD as the appropriate indane, a mixture of regioisomers was obtained. The regioisomers were separated via flash chromatography using heptane and EtOAc as eluents. The regioisomer eluting earlier was collected as Preparation 19aE. ¹H NMR (500 MHz, DMSO-d₆) δ ppm: 7.47 (d, 2H), 7.40 (t, 2H), 7.33 (t, 1H), 7.27 (d, 1H), 7.23 (d, 1H), 6.96 (dd, 1H), 6.95 (s, 1H), 5.12 (s, 2H), 3.95 (t, 4H), 2.08/1.19 (t+d, 4H), 2.03/1.85 (t+d, 4H). HRMS calculated for C₂₃H₂₃BrO₃: 426.0831; found 427.0900 (M+H).

### Preparation 19aF 6′-(benzyloxy)-2′-bromospiro[cyclohexane-1,1′-inden]-4-one

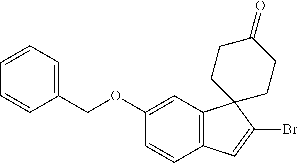 [SMILES: O=C1CCC2(CC1)C(Br)=Cc1ccc(OCc3ccccc3)cc12]

Using General procedure 9 and Preparation 19aE as the appropriate ketal, Preparation 19aF was obtained. ¹H NMR (500 MHz, DMSO-d₆) δ ppm: 7.54 (d, 1H), 7.47 (d, 2H), 7.39 (t, 2H), 7.33 (t, 1H), 7.29 (d, 1H), 7.01 (s, 1H), 6.97 (dd, 1H), 5.14 (s, 2H), 2.91/2.47 (dd+dt, 4H), 2.18/1.62 (td+dt, 4H). HRMS calculated for C₂₁H₁₉BrO₂: 382.0569; found 382.05629 (M+).

### Preparation 19aG (1s,4s)-6′-(benzyloxy)-2′-bromo-4-(3-chloroanilino)spiro[cyclohexane-1,1′-indene]-4-carbonitrile

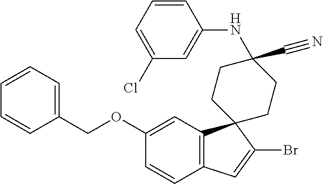 [SMILES: N#C[C@]1(Nc2cccc(Cl)c2)CC[C@]2(CC1)C(Br)=Cc1ccc(OCc3ccccc3)cc12]

Using General procedure 11 and Preparation 19aF as the appropriate ketone and 3-chloroaniline as the appropriate aniline, a mixture of diastereoisomers was obtained. The diastereoisomers were separated by flash chromatography using heptane and EtOAc as eluents. The diastereoisomer eluting earlier was collected as Preparation 19aG. ¹H NMR (500 MHz, DMSO-d₆) δ ppm: 7.50 (dm, 2H), 7.41 (tm, 2H), 7.35 (tm, 1H), 7.28 (d, 1H), 7.26 (d, 1H), 7.24 (t, 1H), 6.97 (dd, 1H), 6.96 (s, 1H), 6.92 (t, 1H), 6.88 (dm, 1H), 6.79 (dm, 1H), 6.57 (s, 1H), 5.17 (s, 2H), 2.51/2.41 (d+tm, 4H), 2.06/1.27 (td+d, 4H). HRMS calculated for C₂₈H₂₄BrClN₂O: 518.076; found 519.0821 (M+H).

### Preparation 19aH (1s,4s)-6′-(benzyloxy)-2′-bromo-4-(3-chloroanilino)spiro[cyclohexane-1,1′-indene]-4-carboxamide

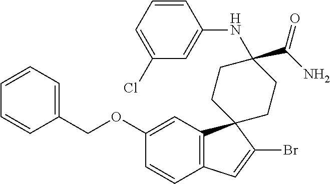 [SMILES: NC(=O)[C@]1(Nc2cccc(Cl)c2)CC[C@]2(CC1)C(Br)=Cc1ccc(OCc3ccccc3)cc12]

Using General procedure 12a and Preparation 19aG as the appropriate nitrile, Preparation 19aH was obtained. ¹H NMR (500 MHz, DMSO-d₆) δ ppm: 7.53-7.30 (m, 5H), 7.42 (d, 1H), 7.34/7.25 (br+br, 2H), 7.25 (d, 1H), 7.12 (t, 1H), 6.95 (dd, 1H), 6.92 (s, 1H), 6.69 (t, 1H), 6.62 (dm, 1H), 6.60 (dm, 1H), 6.22 (s, 1H), 5.12 (s, 2H), 2.45/2.07 (td+d, 4H), 2.10/0.94 (br t+d, 4H). HRMS calculated for C₂₈H₂₆BrClN₂O₂: 536.0866; found 537.0938 (M+H).

### Preparation 19aI (1s,4s)-6′-(benzyloxy)-2′-bromo-4-(3-chloroanilino)spiro[cyclohexane-1,1′-indene]-4-carboxylic acid

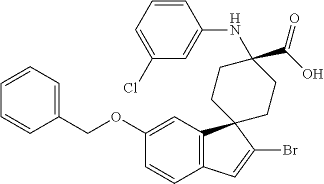 [SMILES: O=C(O)[C@]1(Nc2cccc(Cl)c2)CC[C@]2(CC1)C(Br)=Cc1ccc(OCc3ccccc3)cc12]

Using General procedure 13 and Preparation 19aH as the appropriate amide, Preparation 19aI was obtained. ¹H NMR (500 MHz, DMSO-d₆) δ ppm: 12.83 (br s, 1H), 7.48 (d, 2H), 7.40 (t, 2H), 7.34 (d, 1H), 7.33 (t, 1H), 7.26 (d, 1H), 7.07 (t, 1H), 6.96 (dd, 1H), 6.93 (s, 1H), 6.61 (dd, 1H), 6.56 (dd, 1H), 6.54 (dd, 1H), 6.38 (br s, 1H), 5.12 (s, 2H), 2.35/2.20 (t+d, 4H), 2.16/0.96 (t+d, 4H). HRMS calculated for C₂₈H₂₅BrClNO₃: 537.0706; found 538.0786 (M+H).

### Preparation 19aJ methyl (1s,4s)-6′-(benzyloxy)-2′-bromo-4-(3-chloroanilino)spiro[cyclohexane-1,1′-indene]-4-carboxylate

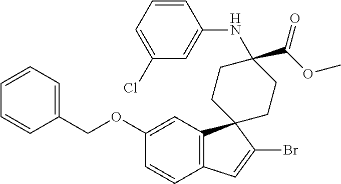 [SMILES: COC(=O)[C@]1(Nc2cccc(Cl)c2)CC[C@]2(CC1)C(Br)=Cc1ccc(OCc3ccccc3)cc12]

Using General procedure 17a and Preparation 19aI as the appropriate amino acid, Preparation 19aJ was obtained. ¹H NMR (500 MHz, DMSO-d₆) δ ppm: 7.50-7.32 (m, 5H), 7.30 (d, 1H), 7.27 (d, 1H), 7.09 (t, 1H), 6.97 (dd, 1H), 6.94 (s, 1H), 6.60 (t, 1H), 6.59 (dm, 1H), 6.48 (s, 1H), 6.46 (dm, 1H), 5.13 (s, 2H), 3.68 (s, 3H), 2.35/2.23 (m+m, 4H), 2.17/0.98 (m+m, 4H). HRMS calculated for C₂₉H₂₇BrClNO₃: 551.0863; found 552.0935 (M+H).

### Preparation 19aK methyl (1r,4R)-6′-(benzyloxy)-4-(3-chloroanilino)-2′-{(2R)-3-[(4-methoxyphenyl)methoxy]-2-methylpropyl}spiro[cyclohexane-1,1′-indene]-4-carboxylate

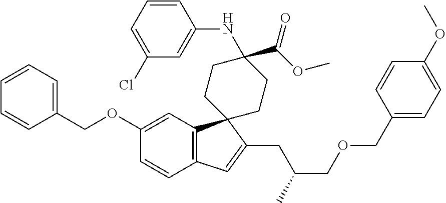 [SMILES: COc1ccc(COC[C@H](C)CC2=Cc3ccc(OCc4ccccc4)cc3[C@]23CC[C@@](Nc2cccc(Cl)c2)(C(=O)OC)CC3)cc1]

Using General procedure 27a and Preparation 19aJ as the appropriate 2-bromo-indene derivative and Preparation 3d as the appropriate Zn reagent, Preparation 19aK was obtained. LRMS calculated for C₄₁H₄₄ClNO₅: 665; found 666 (M+H). ¹H NMR (400 MHz, DMSO-d₆) δ ppm: 7.50-7.45 (m, 2H), 7.44-7.38 (m, 2H), 7.38-7.31 (m, 1H), 7.27 (d, J=2.2 Hz, 1H), 7.24-7.19 (m, 2H), 7.16 (d, J=8.2 Hz, 1H), 7.10 (t, J=8.1 Hz, 1H), 6.90 (dd, J=8.2, 2.2 Hz, 1H), 6.89-6.84 (m, 2H), 6.64 (t, J=2.1 Hz, 1H), 6.62-6.58 (m, 1H), 6.50-6.46 (m, 1H), 6.42 (s, 1H), 6.37-6.34 (m, 1H), 5.11 (s, 2H), 4.42-4.34 (m, 2H), 3.73 (s, 3H), 3.69 (s, 3H), 3.36-3.24 (m, 2H), 2.41-2.27 (m, 3H), 2.22-2.01 (m, 5H), 1.99-1.89 (m, 1H), 0.93 (d, J=6.6 Hz, 3H), 0.90-0.82 (m, 2H).

### Preparation 19aL methyl (1r,4R)-6′-(benzyloxy)-4-(3-chloroanilino)-2′-[(2R)-3-hydroxy-2-methylpropyl]spiro[cyclohexane-1,1′-indene]-4-carboxylate

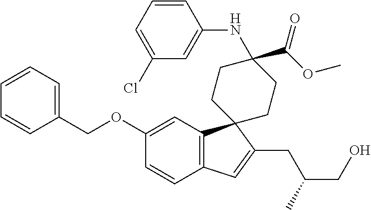 [SMILES: COC(=O)[C@]1(Nc2cccc(Cl)c2)CC[C@]2(CC1)C(C[C@@H](C)CO)=Cc1ccc(OCc3ccccc3)cc12]

Using General procedure 28b and Preparation 19aK as the appropriate PMB derivative, Preparation 19aL was obtained. LRMS calculated for C₃₃H₃₆ClNO₄: 545; found 546 (M+H). ¹H NMR (400 MHz, DMSO-d₆) δ ppm: 7.51-7.45 (m, 2H), 7.45-7.38 (m, 2H), 7.38-7.31 (m, 1H), 7.27 (d, J=2.2 Hz, 1H), 7.18 (d, J=8.2 Hz, 1H), 7.10 (t, J=8.1 Hz, 1H), 6.90 (dd, J=8.2, 2.2 Hz, 1H), 6.65-6.57 (m, 2H), 6.50-6.45 (m, 1H), 6.42 (s, 1H), 6.39-6.36 (m, 1H), 5.11 (s, 2H), 4.53 (t, J=5.2 Hz, 1H), 3.69 (s, 3H), 3.40-3.24 (m, 2H), 2.43-2.27 (m, 3H), 2.24-2.02 (m, 4H), 2.01-1.81 (m, 2H), 0.95-0.81 (m, 5H).

### Preparation 19aM methyl (1r,2′S,4S)-6′-(benzyloxy)-4-(3-chloroanilino)-2′-[(2R)-3-hydroxy-2-methylpropyl]-2′,3′-dihydrospiro[cyclohexane-1,1′-indene]-4-carboxylate

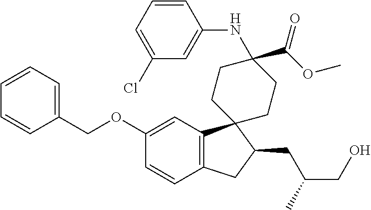 [SMILES: COC(=O)[C@]1(Nc2cccc(Cl)c2)CC[C@@]2(CC1)c1cc(OCc3ccccc3)ccc1C[C@@H]2C[C@@H](C)CO]

and

### Preparation 19bM methyl (1r,2′R,4R)-6′-(benzyloxy)-4-(3-chloroanilino)-2′-[(2R)-3-hydroxy-2-methylpropyl]-2′,3′-dihydrospiro[cyclohexane-1,1′-indene]-4-carboxylate

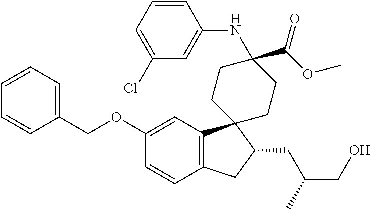 [SMILES: COC(=O)[C@]1(Nc2cccc(Cl)c2)CC[C@@]2(CC1)c1cc(OCc3ccccc3)ccc1C[C@H]2C[C@@H](C)CO]

Using General procedure 19 and Preparation 19aL as the appropriate indene, a mixture of diastereoisomers was obtained. The diastereoisomers were separated by chiral chromatography.

Column: AD, 100×500 mm, 20 μm. Eluents: 30:70 iPrOH/heptane. The diastereoisomer eluting earlier was collected as Preparation 19bM. ¹H NMR (400 MHz, DMSO-d₆) δ ppm: 7.47 (m, 2H), 7.42-7.39 (m, 2H), 7.36-7.32 (m, 1H), 7.11-7.05 (m, 2H), 6.96 (d, 1H), 6.80 (dd, 1H), 6.60-6.56 (m, 2H), 6.47-6.44 (m, 1H), 6.29 (s, 1H), 5.07 (s, 2H), 4.39 (br s, 1H), 3.65 (s, 3H), 3.41 (m, 1H), 3.19 (m, 1H), 2.89/2.36 (m+m, 2H), 2.09 (m, 1H), 2.46-1.40 (m, 8H), 1.59 (m, 1H), 1.27/1.00 (m+m, 2H), 0.91 (d, 3H). LRMS calculated for C₃₃H₃₈ClNO₄: 547.25; found 548.4 (M+H).

The diastereoisomer eluting later was collected as Preparation 19aM. ¹H NMR (500 MHz, DMSO-d₆) δ ppm: 7.45 (m, 2H), 7.39 (m, 2H), 7.33 (m, 1H), 7.09 (d, 1H), 7.06 (t, 1H), 6.95 (d, 1H), 6.79 (dd, 1H), 6.59 (t, 1H), 6.56 (dd, 1H), 6.54 (dd, 1H), 6.30 (s, 1H), 5.07 (s, 2H), 4.44 (br s, 1H), 3.65 (s, 3H), 3.21 (d, 2H), 2.86/2.41 (m+m, 2H), 2.40-1.32 (m, 8H), 2.08 (m, 1H), 1.54 (m, 1H), 1.33/1.04 (m+m, 2H), 0.84 (d, 3H). HRMS calculated for C₃₃H₃₈ClNO₄: 547.249; found 548.25578 (M+H).

### Preparation 19aN methyl (1r,2′S,4S)-6′-(benzyloxy)-4-(3-chloroanilino)-2′-{(2R)-2-methyl-3-[(thieno[3,2-b]pyridin-7-yl)oxy]propyl}-2′,3′-dihydrospiro[cyclohexane-1,1′-indene]-4-carboxylate

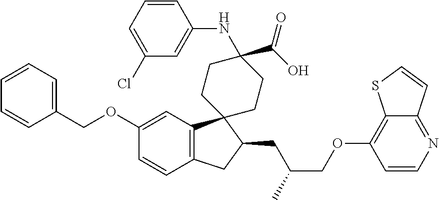 [SMILES: C[C@@H](COc1ccnc2ccsc12)C[C@H]1Cc2ccc(OCc3ccccc3)cc2[C@]12CC[C@@](Nc1cccc(Cl)c1)(C(=O)O)CC2]

Preparation 19aM (955 mg, 1.74 mmol), thieno[3,2-b]pyridin-7-ol (527 mg, 3.48 mmol) and PPh₃ (914 mg, 3.48 mmol) were dissolved in dry THE (17 mL) and cooled to 0° C. 40% DEAD solution in toluene (1.52 mL, 3.48 mmol) was added and the mixture was stirred at 0° C. for 2 h. Then it was diluted with water and sat. aq. NaHCO₃ solution. It was extracted with EtOAc. The combined organic layers were washed with brine, dried over Na₂SO₄, filtered and the filtrate was concentrated under reduced pressure. The crude product was purified via flash chromatography using heptane and EtOAc as eluents to obtain Preparation 19aN. ¹H NMR (500 MHz, DMSO-d₆) δ ppm: 8.50 (d, 1H), 8.00 (d, 1H), 7.50 (d, 1H), 7.48-6.42 (m, 12H), 6.99 (d, 1H), 6.32 (s, 1H), 4.16/4.10 (dd+dd, 2H), 3.65 (s, 3H), 2.92/2.47 (dd+dd, 2H), 2.48-1.28 (m, 8H), 2.14 (m, 1H), 2.05 (m, 1H), 1.46/1.35 (m+m, 2H), 1.06 (d, 3H). HRMS calculated for C₄₀H₄₁ClN₂O₄S: 680.2476; found 681.25477 (M+H).

### Preparation 19a methyl (1r,2′S,4S)-4-(3-chloroanilino)-6′-hydroxy-2′-{(2R)-2-methyl-3-[(thieno[3,2-b]pyridin-7-yl)oxy]propyl}-2′,3′-dihydrospiro[cyclohexane-1,1′-indene]-4-carboxylate

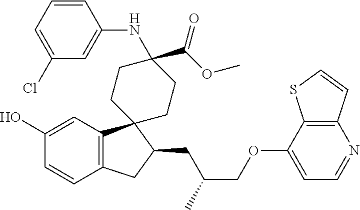 [SMILES: COC(=O)[C@]1(Nc2cccc(Cl)c2)CC[C@@]2(CC1)c1cc(O)ccc1C[C@@H]2C[C@@H](C)COc1ccnc2ccsc12]

Preparation 19aN (845 mg, 1.24 mmol) was dissolved in DCM (25 mL) and EtSH (25 mL). BF₃×Et₂O (3.8 mL, 30.5 mmol) was added and the mixture was stirred at rt overnight. Then it was diluted with sat. aq. NaHCO₃ solution and extracted with DCM. The combined organic layers were dried over Na₂SO₄, filtered and the filtrate was concentrated under reduced pressure. The crude product was purified via flash chromatography using DCM and MeOH as eluents to obtain Preparation 19a. ¹H NMR (500 MHz, DMSO-d₆) δ ppm: 9.09 (s, 1H), 8.51 (d, 1H), 8.01 (d, 1H), 7.50 (d, 1H), 7.05 (t, 1H), 6.99 (d, 1H), 6.96 (d, 1H), 6.82 (d, 1H), 6.60 (t, 1H), 6.56 (dm, 1H), 6.53 (dd, 1H), 6.45 (dm, 1H), 6.32 (s, 1H), 4.16/4.10 (dd+dd, 2H), 3.64 (s, 3H), 2.88/2.41 (dd+dd, 2H), 2.46-1.28 (m, 8H), 2.10 (m, 1H), 2.04 (m, 1H), 1.47/1.34 (m+m, 2H), 1.06 (d, 3H). HRMS calculated for C₃₃H₃₅ClN₂O₄S: 590.2006; found 591.2070 (M+H).

### Preparation 19bN methyl (1r,2′R,4R)-6′-(benzyloxy)-4-(3-chloroanilino)-2′-{(2R)-2-methyl-3-[(thieno[3,2-b]pyridin-7-yl)oxy]propyl}-2′,3′-dihydrospiro[cyclohexane-1,1′-indene]-4-carboxylate

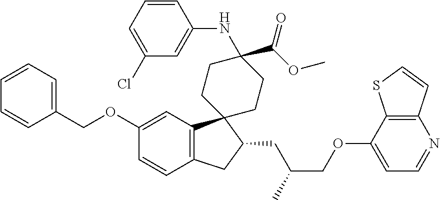 [SMILES: COC(=O)[C@]1(Nc2cccc(Cl)c2)CC[C@@]2(CC1)c1cc(OCc3ccccc3)ccc1C[C@H]2C[C@@H](C)COc1ccnc2ccsc12]

Preparation 19bM (990 mg, 1.81 mmol), thieno[3,2-b]pyridin-7-ol (546 mg, 3.61 mmol) and PPh₃ (947 mg, 3.61 mmol) were dissolved in dry THE (18 mL) and cooled to 0° C. 40% DEAD solution in toluene (1.57 mL, 3.61 mmol) was added and the mixture was stirred at 0° C. for 1 h. Then it was diluted with water and sat. aq. NaHCO₃ solution. It was extracted with EtOAc. The combined organic layers were washed with brine, dried over Na₂SO₄, filtered and the filtrate was concentrated under reduced pressure. The crude product was purified via flash chromatography using heptane and EtOAc as eluents to obtain Preparation 19bN. ¹H NMR (500 MHz, DMSO-d₆) δ ppm: 8.50 (d, 1H), 7.90 (d, 1H), 7.48 (d, 1H), 7.42 (d, 2H), 7.39 (t, 2H), 7.33 (t, 1H), 7.10 (d, 1H), 7.05 (t, 1H), 6.98 (d, 1H), 6.92 (d, 1H), 6.80 (dd, 1H), 6.57 (m, 2H), 6.42 (dd, 1H), 6.22 (s, 1H), 5.06 (s, 2H), 4.23/4.12 (dd+dd, 2H), 3.64 (s, 3H), 2.97/2.52 (dd+dd, 2H), 2.42-1.27 (m, 8H), 2.17 (dd, 1H), 2.12 (m, 1H), 1.67/1.28 (dd+dd, 2H), 1.09 (d, 3H). HRMS calculated for C₄₀H₄₁ClN₂O₄S: 680.2476; found 681.2549 (M+H).

### Preparation 19b methyl (1r,2′R,4R)-4-(3-chloroanilino)-6′-hydroxy-2′-{(2R)-2-methyl-3-[(thieno[3,2-b]pyridin-7-yl)oxy]propyl}-2′,3′-dihydrospiro[cyclohexane-1,1′-indene]-4-carboxylate

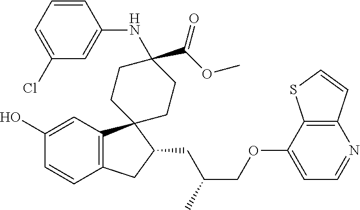 [SMILES: COC(=O)[C@]1(Nc2cccc(Cl)c2)CC[C@@]2(CC1)c1cc(O)ccc1C[C@H]2C[C@@H](C)COc1ccnc2ccsc12]

Preparation 19bN (864 mg, 1.27 mmol) was dissolved in DCM (25 mL) and EtSH (25 mL). BF₃×Et₂O (3.8 mL, 30.5 mmol) was added and the mixture was stirred at rt overnight. Then it was diluted with sat. aq. NaHCO₃ solution and extracted with DCM. The combined organic layers were dried over Na₂SO₄, filtered and the filtrate was concentrated under reduced pressure. The crude product was purified via flash chromatography using heptane and EtOAc as eluents to obtain Preparation 19b. ¹H NMR (500 MHz, DMSO-d₆) δ ppm: 9.09 (s, 1H), 8.50 (d, 1H), 7.90 (d, 1H), 7.47 (d, 1H), 7.04 (t, 1H), 6.99 (d, 1H), 6.96 (d, 1H), 6.83 (d, 1H), 6.57 (m, 2H), 6.53 (dd, 1H), 6.42 (dm, 1H), 6.22 (s, 1H), 4.23/4.12 (dd+dd, 2H), 3.64 (s, 3H), 2.91/2.47 (dd+dd, 2H), 2.42-1.24 (m, 8H), 2.14 (m, 1H), 2.12 (m, 1H), 1.67/1.28 (m+m, 2H), 1.09 (d, 3H). HRMS calculated for C₃₃H₃₅ClN₂O₄S: 590.2006; found 591.2072 (M+H).

### Preparation 20a methyl (1r,2′S,4S)-4-(3-chloroanilino)-5′,6′-dihydroxy-2′-[(2R)-3-hydroxy-2-methylpropyl]-2′,3′-dihydrospiro[cyclohexane-1,1′-indene]-4-carboxylate

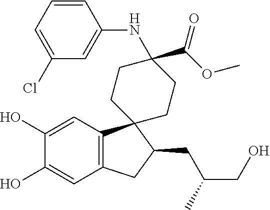 [SMILES: COC(=O)[C@]1(Nc2cccc(Cl)c2)CC[C@@]2(CC1)c1cc(O)c(O)cc1C[C@@H]2C[C@@H](C)CO]

Example 839E (5.00 g, 10.3 mmol, 1 eq) was dissolved in DCM (103 mL) and cooled to 0° C. BBr₃ (2.97 mL, 30.9 mmol, 3 eq) was added in one portion and the mixture was stirred at 0° C. for 30 min. Then MeOH was added and the mixture was concentrated under reduced pressure. MeOH was added again and the mixture was concentrated under reduced pressure. The residue was dissolved in THF and washed with brine. The organic layer was dried over Na₂SO₄, filtered and the filtrate was concentrated under reduced pressure to obtain catechol derivative Preparation 20a (4.84 g, 10.2 mmol, 99%). ¹H NMR (400 MHz, DMSO-d₆) δ ppm: 8.54 (s, 2H), 7.06 (t, 1H), 6.79 (s, 1H), 6.59 (t, 1H), 6.57 (dm, 1H), 6.55 (s, 1H), 6.44 (dm, 1H), 6.28 (s, 1H), 4.43 (t, 1H), 3.64 (s, 3H), 3.21 (m, 2H), 2.74/2.31 (dd+dd, 2H), 2.43-1.15 (m, 8H), 1.98 (m, 1H), 1.53 (m, 1H), 1.33/1.02 (m+m, 2H), 0.83 (d, 3H). HRMS calculated for C₂₆H₃₂ClNO₅: 473.1969; found: 474.2031 (M+H).

### Preparation 20b methyl (1r,2′S,4S)-4-(3-chloroanilino)-5′,6′-dihydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

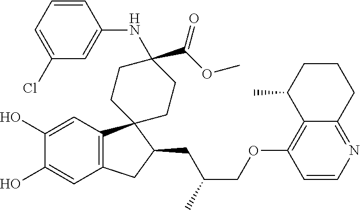 [SMILES: COC(=O)[C@]1(Nc2cccc(Cl)c2)CC[C@@]2(CC1)c1cc(O)c(O)cc1C[C@@H]2C[C@@H](C)COc1ccnc2c1[C@H](C)CCC2]

Example 839 (3.77 g, 5.97 mmol, 1 eq) was dissolved in DCM (60 mL) and cooled to 0° C. BBr₃ (1.72 mL, 17.9 mmol, 3 eq) was added in one portion and the mixture was stirred at 0° C. for 30 min. Partial hydrolysis of the carboxylate ester was also observed. MeOH was added and the mixture was concentrated under reduced pressure. MeOH was added again and the mixture was concentrated under reduced pressure. The residue was dissolved in THE and washed with brine. The organic layer was dried over Na₂SO₄, filtered and the filtrate was concentrated under reduced pressure. The residue was treated as described in General procedure 17a then, instead flash chromatography the crude product was purified via prep RP-HPLC using 25 mM aq. NH₄HCO₃ solution and MeCN as eluents to obtain catechol derivative Preparation 20b (3.25 g, 5.25 mmol, 88%). ¹H NMR (500 MHz, DMSO-d₆) δ ppm: 8.55 (s, 1H), 8.54 (s, 1H), 8.18 (d, 1H), 7.04 (t, 1H), 6.82 (d, 1H), 6.81 (s, 1H), 6.57 (t, 1H), 6.56 (s, 1H), 6.55 (dm, 1H), 6.43 (dm, 1H), 6.30 (s, 1H), 3.93/3.86 (dd+dd, 2H), 3.64 (s, 3H), 3.06 (m, 1H), 2.80/2.34 (dd+dd, 2H), 2.77/2.67 (dm+m, 2H), 2.41-1.20 (m, 14H), 2.02 (m, 1H), 1.98 (m, 1H), 1.07 (d, 3H), 1.03 (d, 3H). HRMS calculated for C₃₆H₄₃ClN₂O₅: 618.2861; found: 619.2909 (M+H).

### EXAMPLES

### Example 1 (1r,4r)-4-(3-bromoanilino)-2′-oxo-1′,2′-dihydrospiro[cyclohexane-1,3′-indole]-4-carboxylic acid

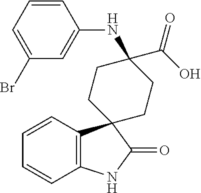 [SMILES: O=C(O)[C@]1(Nc2cccc(Br)c2)CC[C@@]2(CC1)C(=O)Nc1ccccc12]

A solution of 1′H-spiro[cyclohexane-1,3′-indole]-2′,4-dione (137 mg, 0.64 mmol, 1.5 eq) in THE (6 mL), cooled in an ice bath, was treated as described in General procedure 10 using 3-bromoaniline (46 μL, 0.42 mmol, 1 eq) and the mixture stirred at rt for 48 h. The mixture was diluted with water, acidified with 2 M aq. HCl solution and then extracted with DCM. The organic phase was loaded onto a DCM-wet PE-AX cartridge (10 g) and washed successively with DCM, MeOH and eluted with 5% HCOOH in DCM, and then concentrated in vacuo. The residue was suspended in DCM and the precipitate collected by filtration, washed with DCM and dried in vacuo to afford a single diastereoisomer, Example 1 as a white powder (10.7 mg, 0.03 mmol, 6%). LRMS calculated for C₂₀H₁₉N₂O₃Br: 414; found: 415 (M+H). ¹H NMR (400 MHz, DMSO-d₆) δ ppm: 12.69 (br s, 1H), 10.31 (s, 1H), 7.25 (d, J=7.3 Hz, 1H), 7.17 (td, J=7.7, 1.2 Hz, 1H), 7.03 (t, J=8.1 Hz, 1H), 6.97 (td, J=7.6, 1.1 Hz, 1H), 6.85 (t, J=2.1 Hz, 1H), 6.83 (d, J=7.6 Hz, 1H), 6.71 (ddd, J=7.9, 1.9, 0.8 Hz, 1H), 6.65-6.61 (m, 1H), 6.28 (br s, 1H), 2.62-2.54 (m, 2H), 2.03-1.87 (m, 4H), 1.66-1.57 (m, 2H).

### Example 2

### Example 2A 1′-ethylspiro[cyclohexane-1,3′-indole]-2′,4-dione

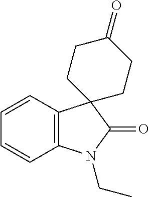 [SMILES: CCN1C(=O)C2(CCC(=O)CC2)c2ccccc21]

To a solution of 1′H-spiro[cyclohexane-1,3′-indole]-2′,4-dione (150 mg, 0.7 mmol, 1 eq) in MeCN (4 mL) was added Cs₂CO₃ (454 mg, 1.39 mmol, 2 eq) and EtI (62 μL, 0.77 mmol, 1.1 eq) and the mixture stirred at rt under a N₂ atmosphere for 18 h. The reaction was diluted with water, extracted with EtOAc and the organic phase dried (MgSO₄) and concentrated in vacuo to afford Example 2A as a yellow solid (97.4 mg, 0.4 mmol, 57%). ¹H NMR (400 MHz, DMSO-d₆) δ ppm: 7.53 (dd, J=7.5, 1.0 Hz, 1H), 7.31 (td, J=7.7, 1.2 Hz, 1H), 7.11 (d, J=7.7, 1H), 7.06 (td, J=7.5, 1.0 Hz, 1H), 3.74 (q, J=7.2 Hz, 2H), 2.88 (ddd, J=15.6, 10.2, 5.9 Hz, 2H), 2.43 (dt, J=14.9, 5.7 Hz, 2H), 2.16 (ddd, J=14.9, 10.1, 5.2 Hz, 2H), 1.98 (dt, J=12.6, 5.9 Hz, 2H), 1.18 (t, J=7.1 Hz, 3H).

### Example 2 (1r,4r)-4-(3-bromoanilino)-1′-ethyl-2′-oxo-1′,2′-dihydrospiro[cyclohexane-1,3′-indole]-4-carboxylic acid

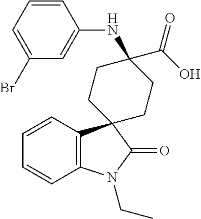 [SMILES: CCN1c2ccccc2[C@]2(CC[C@](Nc3cccc(Br)c3)(C(=O)O)CC2)C1=O]

A solution of Example 2A (97.4 mg, 0.4 mmol, 1 eq) in THE (4 mL) was treated as described in General procedure 10 using 3-bromoaniline (44 μL, 0.4 mmol, 1 eq) and the mixture stirred at rt for 18 h. The mixture was diluted with water, acidified with 2 M aq. HCl solution and then extracted with EtOAc. The organic phase was loaded onto a DCM-wet PE-AX cartridge (10 g) and washed successively with DCM, MeOH and eluted with 5% HCOOH in DCM, and then concentrated in vacuo. Purification by flash chromatography (5 g silica cartridge) eluting with a stepped gradient of 0-40% EtOAc in heptane, followed by trituration with DCM, afforded a single diastereoisomer, Example 2 as a white powder (9.5 mg, 0.02 mmol, 5%). LRMS calculated for C₂₂H₂₃N₂O₃Br: 442; found: 443 (M+H). ¹H NMR (400 MHz, DMSO-d₆) δ ppm: 12.71 (br s, 1H), 7.33-7.24 (m, 2H), 7.08-6.99 (m, 3H), 6.85 (t, J=2.1 Hz, 1H), 6.73-6.68 (m, 1H), 6.65-6.60 (m, 1H), 6.29 (br s, 1H), 3.69 (q, J=7.0 Hz, 2H), 2.64-2.54 (m, 2H), 2.05-1.91 (m, 4H), 1.62-1.53 (m, 2H), 1.14 (t, J=7.1 Hz, 3H).

### Example 3

### Example 3A 1″H-dispiro[1,3-dioxolane-2,1′-cyclohexane-4′,3″-indole]-2″-one

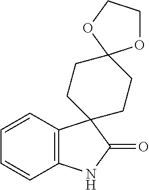 [SMILES: O=C1Nc2ccccc2C12CCC1(CC2)OCCO1]

To a suspension of 1′H-spiro[cyclohexane-1,3′-indole]-2′,4-dione (250 mg, 1.16 mmol, 1 eq) and 4Å molecular sieves in anhydrous toluene (10 mL), was added PTSA (11 mg, 0.06 mmol, 0.05 eq) and ethylene glycol (0.13 mL, 2.32 mmol, 2 eq) and the reaction refluxed for 18 h. The mixture was diluted with EtOAc, washed with sat. aq. NaHCO₃ solution, dried (MgSO₄) and concentrated in vacuo to afford Example 3A as a peach solid (263 mg, 1.01 mmol, 87%). ¹H NMR (400 MHz, DMSO-d₆) δ ppm: 10.35 (s, 1H), 7.31 (d, J=7.4 Hz, 1H), 7.18 (td, J=7.7, 1.2 Hz, 1H), 6.96 (td, J=7.6, 1.1 Hz, 1H), 6.85 (d, J=7.7 Hz, 1H), 3.96-3.91 (m, 4H), 2.13-2.04 (m, 2H), 1.87-1.68 (m, 6H).

### Example 3B 1″,2″-dihydrodispiro[1,3-dioxolane-2,1′-cyclohexane-4′,3″-indole]

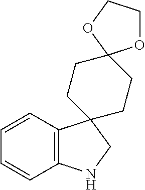 [SMILES: c1ccc2c(c1)NCC21CCC2(CC1)OCCO2]

A solution of Example 3A (160 mg, 0.62 mmol, 1 eq) in anhydrous THE (5 mL), cooled in an ice bath under a N₂ atmosphere, was treated by the dropwise addition of LAH in THE (1 M, 1.85 mL, 1.85 mmol, 3 eq). After stirring for 30 min, the reaction was allowed to warm to rt and then refluxed for 2 h. The mixture was allowed to cool to rt, carefully quenched with minimal water and then diluted with EtOAc. The mixture was dried (MgSO₄) and concentrated in vacuo to afford Example 3B as an off-white solid (135.6 mg, 0.55 mmol, 89%). ¹H NMR (400 MHz, DMSO-d₆) δ ppm: 6.95-6.87 (m, 2H), 6.53 (td, J=7.4, 1.1 Hz, 1H), 6.47 (d, J=7.7 Hz, 1H), 5.48 (app s, 1H), 3.94-3.85 (m, 4H), 3.32 (d, J=2.1 Hz, 2H), 1.84-1.72 (m, 2H), 1.71-1.57 (m, 6H).

### Example 3C 1′,2′-dihydrospiro[cyclohexane-1,3′-indole]-4-one

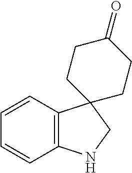 [SMILES: O=C1CCC2(CC1)CNc1ccccc12]

A solution of Example 3B (135 mg, 0.55 mmol, 1 eq) in acetone (5 mL) was treated with 2 M aq. HCl (1.38 mL, 2.75 mmol, 5 eq) and then stirred at 45° C. for 2 h. The resulting solid was collected by filtration and washed with acetone to afford Example 3C as a white powder (62.8 mg, 0.31 mmol, 56%). ¹H NMR (400 MHz, DMSO-d₆) δ ppm: 10.78 (br s, 1H), 7.48-7.44 (m, 1H), 7.37-7.24 (m, 3H), 3.82 (s, 2H), 2.68-2.56 (m, 2H), 2.32-2.23 (m, 2H), 2.17 (td, J=13.4, 4.6 Hz, 2H), 2.06-1.97 (m, 2H).

### Example 3 4-(3-bromoanilino)-1′,2′-dihydrospiro[cyclohexane-1,3′-indole]-4-carboxylic acid

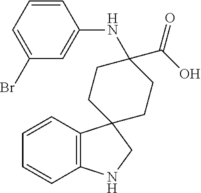 [SMILES: O=C(O)C1(Nc2cccc(Br)c2)CCC2(CC1)CNc1ccccc12]

A solution of Example 3C (62 mg, 0.31 mmol, 1 eq) in THE (3 mL) was treated as described in General procedure 10 using 3-bromoaniline (34 μL, 0.31 mmol, 1 eq) and stirred at rt for 18 h. The reaction was diluted with water, acidified with 2 M aq. HCl solution and extracted with EtOAc. The organic phase was concentrated, dissolved in minimal DCM and loaded onto a DCM-wet PE-AX cartridge (5 g), washing successively with DCM, MeOH and then eluted with 5% HCOOH in DCM and concentrated in vacuo. Further purification by flash chromatography (5 g silica cartridge) eluting with a stepped gradient of 0-2% MeOH in DCM afforded a single diastereoisomer, Example 3 as a white powder (3.3 mg, 0.01 mmol, 2%). LRMS calculated for C₂₀H₂₁N₂O₂Br: 400; found: 401 (M+H). ¹H NMR (400 MHz, DMSO-d₆) δ ppm: 12.75 (br s, 1H), 7.00 (t, J=8.1 Hz, 1H), 6.96-6.89 (m, 2H), 6.82 (t, J=2.1 Hz, 1H), 6.68 (d, J=7.9 Hz, 1H), 6.64-6.59 (m, 1H), 6.55 (td, J=7.4, 1.1 Hz, 1H), 6.48 (d, J=7.5 Hz, 1H), 6.14 (br s, 1H), 5.49 (s, 1H), 3.28 (s, 2H), 2.41-2.30 (m, 2H), 1.81-1.57 (m, 6H).

### Example 4

### Example 4A 1″-methyldispiro[1,3-dioxolane-2,1′-cyclohexane-4′,3″-indole]-2″-one

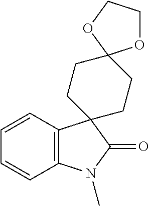 [SMILES: CN1C(=O)C2(CCC3(CC2)OCCO3)c2ccccc21]

To a solution of Example 3A (217 mg, 0.84 mmol, 1 eq) in MeCN (5 mL) was added Cs₂CO₃ (545 mg, 1.67 mmol, 2 eq) followed by MeI (57 μL, 0.92 mmol, 1.1 eq) and the reaction stirred at rt for 18 h. A further portion of MeI (25 μL, 0.40 mmol, 0.48 eq) was added and the mixture continued to stir at rt for 3 h. The reaction was diluted with water and extracted with EtOAc. The organic phase was washed with water and brine, dried (MgSO₄) and concentrated in vacuo to afford Example 4A as a yellow oil (228 mg, 0.83 mmol, 99%). LRMS calculated for C₁₆H₁₉NO₃: 273; found: 274 (M+H). ¹H NMR (400 MHz, DMSO-d₆) δ ppm: 7.36 (d, J=7.6 Hz, 1H), 7.29 (td, J=7.7, 1.2 Hz, 1H), 7.07-7.00 (m, 2H), 3.97-3.90 (m, 4H), 3.13 (s, 3H), 2.16-2.06 (m, 2H), 1.87-1.70 (m, 6H).

### Example 4B 1″-methyl-2″H-dispiro[1,3-dioxolane-2,1′-cyclohexane-4′,3″-indole]

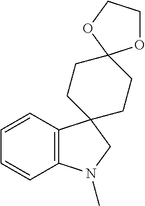 [SMILES: CN1CC2(CCC3(CC2)OCCO3)c2ccccc21]

A solution of Example 4A (228 mg, 0.83 mmol, 1 eq) in anhydrous THE (10 mL) cooled in an ice bath under a N₂ atmosphere, was treated by the dropwise addition of LAH in THE (1 M, 2.5 mL, 2.5 mmol, 3 eq). After stirring for 30 min, the reaction was allowed to warm to rt, refluxed for 2 h and then stirred at rt for 18 h. The reaction was carefully quenched with minimal water and then diluted with EtOAc. The mixture was dried (MgSO₄) and concentrated in vacuo. Purification by flash chromatography (5 g silica cartridge) eluting with a stepped gradient of 0-10% EtOAc in heptane afforded Example 4B as a white solid (78 mg, 0.3 mmol, 36%). LRMS calculated for C₁₆H₂₁NO₂: 259; found: 260 (M+H). ¹H NMR (400 MHz, DMSO-d₆) δ ppm: 7.01 (td, J=7.6, 1.3 Hz, 1H), 6.96 (dd, J=7.5, 1.3 Hz, 1H), 6.61 (td, J=7.4, 1.0 Hz, 1H), 6.49 (d, J=7.6 Hz, 1H), 3.94-3.85 (m, 4H), 3.18 (s, 2H), 2.70 (s, 3H), 1.85-1.74 (m, 2H), 1.73-1.57 (m, 6H).

### Example 4C: 1′-methyl-2′H-spiro[cyclohexane-1,3′-indole]-4-one

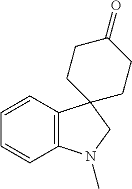 [SMILES: CN1CC2(CCC(=O)CC2)c2ccccc21]

A solution of Example 4B (78 mg, 0.3 mmol, 1 eq) in acetone (3 mL) was treated as described in General procedure 9 and stirred at 45° C. for 3 h. The reaction was cooled to rt, diluted with water and extracted with EtOAc. The organic phase was dried (MgSO₄) and concentrated in vacuo to afford Example 4C as a beige solid (43 mg, 0.2 mmol, 66%). LRMS calculated for C₁₄H₁₇NO: 215; found: 216 (M+H). ¹H NMR (400 MHz, DMSO-d₆) δ ppm: 7.10-7.02 (m, 2H), 6.63 (td, J=7.4, 1.0 Hz, 1H), 6.54 (d, J=7.6 Hz, 1H), 3.39 (s, 2H), 2.76 (s, 3H), 2.62-2.52 (m, 2H), 2.28-2.20 (m, 2H), 2.03 (td, J=13.1, 4.5 Hz, 2H), 1.96-1.88 (m, 2H).

### Example 4 4-(3-bromoanilino)-1′-methyl-1′,2′-dihydrospiro[cyclohexane-1,3′-indole]-4-carboxylic acid

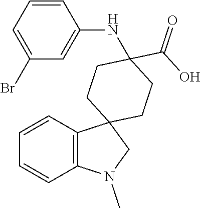 [SMILES: CN1CC2(CCC(Nc3cccc(Br)c3)(C(=O)O)CC2)c2ccccc21]

A solution of Example 4C (43 mg, 0.2 mmol, 1 eq) in anhydrous THE (4 mL) was treated as described in General procedure 10 using 3-bromoaniline (22 μL, 0.2 mmol, 1 eq) and then stirred at rt for 18 h. The reaction was diluted with water, acidified with 2 M aq. HCl solution and extracted into EtOAc. The organic phase was loaded onto a DCM-wet PE-AX cartridge (5 g) and washed successively with DCM, MeOH and then eluted with 5% HCOOH in DCM. Further purification by flash chromatography (10 g silica cartridge), eluting with a stepped gradient of 0-30% EtOAc in heptane, was followed by trituration with iPrOH and then flash chromatography (5 g silica cartridge) eluting with a gradient of 0-1% MeOH in DCM. The material was dissolved in DCM, loaded onto a DCM-wet PE-AX cartridge (5 g) and washed successively with DCM, MeOH and eluted with 5% HCOOH in DCM, and then concentrated in vacuo to afford a single diastereoisomer, Example 4 as a cream powder (6.9 mg, 0.02 mmol, 8%). LRMS calculated for C₂₁H₂₃N₂O₂Br: 414; found: 415 (M+H). ¹H NMR (400 MHz, DMSO-d₆) δ ppm: 7.06-6.95 (m, 3H), 6.82 (t, J=2.1 Hz, 1H), 6.69 (dd, J=7.4, 1.8 Hz, 1H), 6.65-6.60 (m, 2H), 6.50 (d, J=7.6 Hz, 1H), 3.14 (s, 2H), 2.70 (s, 3H), 2.41-2.33 (m, 2H), 1.83-1.61 (m, 6H).

### Example 5

### Example 5A 5″-iodo-1″H-dispiro[1,3-dioxolane-2,1′-cyclohexane-4′,3″-indole]-2″-one

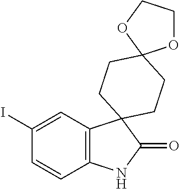 [SMILES: O=C1Nc2ccc(I)cc2C12CCC1(CC2)OCCO1]

To a solution of Example 3A (69 mg, 0.27 mmol, 1 eq) in glacial AcOH (0.27 mL), was added NIS (72 mg, 0.32 mmol, 1.2 eq) and the mixture stirred at rt for 2 h. A further portion of NIS (30 mg, 0.13 mmol, 0.5 eq) was added and the reaction stirred at rt for 2 h. The reaction was diluted with water (2 mL) and the precipitate was collected by filtration and washed with water. The solid was dissolved in DCM, dried (MgSO₄) and concentrated in vacuo. Purification by flash chromatography (5 g silica cartridge) eluting with a stepped gradient of 0-30% EtOAc in heptane, followed by trituration with Et₂O, afforded Example 5A as an off-white powder (47 mg, 0.12 mmol, 45%). LRMS calculated for C₁₅H₁₆NO₃I: 385; found: 386 (M+H). ¹H NMR (400 MHz, DMSO-d₆) δ ppm: 10.49 (s, 1H), 7.56-7.52 (m, 2H), 6.71 (d, J=8.0 Hz, 1H), 3.96-3.89 (m, 4H), 2.15-2.05 (m, 2H), 1.82-1.65 (m, 6H).

### Example 5B 5″-iodo-1″-methyldispiro[1,3-dioxolane-2,1′-cyclohexane-4′,3″-indole]-2″-one

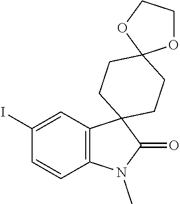 [SMILES: CN1C(=O)C2(CCC3(CC2)OCCO3)c2cc(I)ccc21]

To a suspension of Example 5A (47 mg, 0.12 mmol, 1 eq) and Cs₂CO₃ (80 mg, 0.24 mmol, 2 eq) in MeCN (2 mL) was added MeI (23 mg, 0.16 mmol, 1.3 eq) and the mixture stirred at rt for 5 h. The reaction was diluted with water and the precipitate collected by filtration, dissolved in DCM, dried (MgSO₄) and concentrated in vacuo to afford Example 5B as a beige powder (44.5 mg, 0.11 mmol, 91%). LRMS calculated for C₁₆H₁₈NO₃I: 399; found: 400 (M+H). ¹H NMR (400 MHz, DMSO-d₆) δ ppm: 7.65 (dd, J=8.2, 1.7 Hz, 1H), 7.60 (d, J=1.7 Hz, 1H), 6.90 (d, J=8.1 Hz, 1H), 3.97-3.90 (m, 4H), 3.10 (s, 3H), 2.17-2.07 (m, 2H), 1.79 (app t, J=6.2 Hz, 4H), 1.74-1.66 (m, 2H).

### Example 5C 5′-iodo-1′-methylspiro[cyclohexane-1,3′-indole]-2′,4-dione

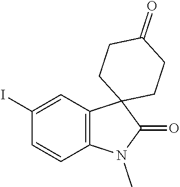 [SMILES: CN1C(=O)C2(CCC(=O)CC2)c2cc(I)ccc21]

A solution of Example 5B (44.5 mg, 0.11 mmol, 1 eq) in acetone (2 mL) was treated as described in General procedure 9 and stirred at 50° C. for 1 h. The reaction was cooled to rt, diluted with water and extracted with DCM. The organic phase was dried (MgSO₄) and concentrated in vacuo to afford Example 5C as a colourless oil which solidified on standing (39 mg, 0.11 mmol, 98%). LRMS calculated for C₁₄H₁₄NO₂I: 355; found: 356 (M+H). ¹H NMR (400 MHz, DMSO-d₆) δ ppm: 7.85 (d, J=1.7 Hz, 1H), 7.67 (dd, J=8.2, 1.7 Hz, 1H), 6.92 (d, J=8.2 Hz, 1H), 3.15 (s, 3H), 2.89 (ddd, J=16.0, 10.9, 5.9 Hz, 2H), 2.42-2.32 (m, 2H), 2.22 (ddd, J=15.8, 11.0, 5.1 Hz, 2H), 2.02-1.92 (m, 2H).

### Example 5 (1r,4r)-4-(3-bromoanilino)-5′-iodo-1′-methyl-2′-oxo-1′,2′-dihydrospiro[cyclohexane-1,3′-indole]-4-carboxylic acid

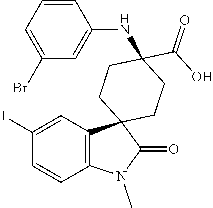 [SMILES: CN1c2ccc(I)cc2[C@]2(CC[C@](Nc3cccc(Br)c3)(C(=O)O)CC2)C1=O]

A solution of Example 5C (39 mg, 0.11 mmol, 1 eq) in anhydrous THE (3 mL) was treated as described in General procedure 10 using 3-bromoaniline (12 μL, 0.11 mmol, 1 eq) and the mixture stirred at rt for 18 h. The reaction was diluted with water, acidified with 2 M aq. HCl solution and extracted with DCM. The organic phase was concentrated, dissolved in minimal DCM and loaded onto a DCM-wet PE-AX cartridge (5 g), washing successively with DCM, MeOH and then eluted with 5% HCOOH in DCM and concentrated in vacuo. Further purification by flash chromatography (5 g silica cartridge) eluting with a stepped gradient of 0-5% MeOH in DCM, followed by preparative HPLC at pH 4 afforded a single diastereoisomer, Example 5 as a white powder (4 mg, 0.01 mmol, 6%). LRMS calculated for C₂₁H₂₀N₂O₃BrI: 554; found: 555 (M+H). ¹H NMR (400 MHz, DMSO-d₆) δ ppm: 12.73 (br s, 1H), 7.63 (dd, J=8.1, 1.7 Hz, 1H), 7.57 (d, J=1.7 Hz, 1H), 7.03 (t, J=8.0 Hz, 1H), 6.87 (d, J=8.2 Hz, 1H), 6.85 (t, J=2.0 Hz, 1H), 6.71 (d, J=7.8 Hz, 1H), 6.64 (dd, J=8.4 Hz, 1H), 6.21 (br s, 1H), 3.09 (s, 3H), 2.61-2.52 (m, 2H), 2.03-1.90 (m, 4H), 1.63-1.55 (m, 2H).

### Example 6

### Example 6A 5″-bromo-1″H-dispiro[1,3-dioxolane-2,1′-cyclohexane-4′,3″-indole]-2″-one

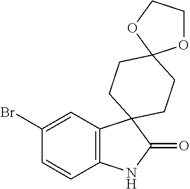 [SMILES: O=C1Nc2ccc(Br)cc2C12CCC1(CC2)OCCO1]

To a solution of Example 3A (597 mg, 2.3 mmol, 1 eq) in MeCN (10 mL), cooled in an ice bath, was added NBS (533 mg, 2.99 mmol, 1.3 eq) portionwise and then cooling maintained for 3 h. The reaction was diluted with water and extracted with DCM. The organic phase was dried (MgSO₄), concentrated in vacuo and triturated with Et₂O. The precipitate was collected by filtration and washed with Et₂O to afford Example 6A as a cream powder (288 mg, 0.85 mmol, 36%). LRMS calculated for C₁₅H₁₆NO₃Br: 337; found: 338 (M+H). ¹H NMR (400 MHz, DMSO-d₆) δ ppm: 10.51 (s, 1H), 7.43 (d, J=2.0 Hz, 1H), 7.38 (dd, J=8.2, 2.0 Hz, 1H), 6.82 (d, J=8.2 Hz, 1H), 4.00-3.89 (m, 4H), 2.15-2.06 (m, 2H), 1.87-1.65 (m, 6H).

### Example 6B 5″-bromo-1″-methyldispiro[1,3-dioxolane-2,1′-cyclohexane-4′,3″-indole]-2″-one

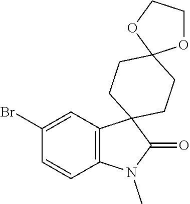 [SMILES: CN1C(=O)C2(CCC3(CC2)OCCO3)c2cc(Br)ccc21]

To a suspension of Example 6A (288 mg, 0.85 mmol, 1 eq) and Cs₂CO₃ (555 mg, 1.7 mmol, 2 eq) in MeCN (5 mL) was added MeI (69 μL, 1.11 mmol, 1.3 eq) and the mixture stirred at rt for 2 h. The reaction was diluted with water, extracted with DCM and the organic phase dried (MgSO₄) and concentrated in vacuo. Purification by flash chromatography (20 g silica cartridge) eluting with a gradient of 0-20% EtOAc in heptane afforded Example 6B as a white powder (212 mg, 0.6 mmol, 70%). LRMS calculated for C₁₆H₁₈NO₃Br: 351; found: 352 (M+H). ¹H NMR (400 MHz, DMSO-d₆) δ ppm: 7.52-7.47 (m, 2H), 7.01 (dd, J=7.9, 0.7 Hz, 1H), 3.97-3.91 (m, 4H), 3.12 (s, 3H), 2.18-2.09 (m, 2H), 1.80 (app t, J=6.2 Hz, 4H), 1.76-1.67 (m, 2H).

### Example 6C 5′-bromo-1′-methylspiro[cyclohexane-1,3′-indole]-2′,4-dione

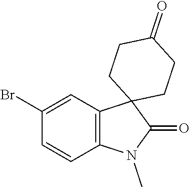 [SMILES: CN1C(=O)C2(CCC(=O)CC2)c2cc(Br)ccc21]

A solution of Example 6B (50 mg, 0.14 mmol, 1 eq) in acetone (3 mL) was treated as described in General procedure 9 and the mixture stirred at 45° C. for 3 h. The reaction was allowed to cool to rt and then partitioned between DCM and water. The organic phase was dried (MgSO₄) and concentrated in vacuo to afford Example 6C as a colourless oil which solidified on standing (36 mg, 0.12 mmol, 82%). ¹H NMR (400 MHz, DMSO-d₆) δ ppm: 7.75 (d, J=2.0 Hz, 1H), 7.51 (dd, J=8.3, 2.0 Hz, 1H), 7.04 (d, J=8.3 Hz, 1H), 3.16 (s, 3H), 2.90 (ddd, J=16.0, 10.8, 5.9 Hz, 2H), 2.39 (dt, J=15.2, 5.3 Hz, 2H), 2.23 (ddd, J=13.7, 10.9, 5.1 Hz, 2H), 2.03-1.94 (m, 2H).

### Example 6 (1r,4r)-5′-bromo-4-(3-bromoanilino)-1′-methyl-2′-oxo-1′,2′-dihydrospiro[cyclohexane-1,3′-indole]-4-carboxylic acid

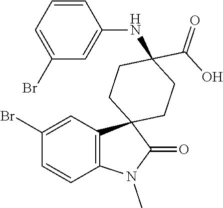 [SMILES: CN1c2ccc(Br)cc2[C@]2(CC[C@](Nc3cccc(Br)c3)(C(=O)O)CC2)C1=O]

A solution of Example 6C (36 mg, 0.12 mmol, 1 eq) in THE (2 mL) was treated as described in General procedure 10 using 3-bromoaniline (13 μL, 0.12 mmol, 1 eq) and stirred at rt for 18 h. The reaction was diluted with water, acidified with 2 M aq. HCl solution and extracted with EtOAc. The organic phase was loaded onto a DCM-wet PE-AX cartridge (5 g), washing successively with DCM, MeOH and then eluted with 5% HCOOH in DCM and concentrated in vacuo. Further purification by preparative HPLC at pH 9 afforded a single diastereoisomer, Example 6 as an off-white powder (8.1 mg, 0.02 mmol, 13%). LRMS calculated for C₂₁H₂₀N₂O₃Br₂: 506; found: 507 (M+H). ¹H NMR (400 MHz, DMSO-d₆) δ ppm: 7.49-7.46 (m, 2H), 7.02-6.96 (m, 2H), 6.88 (t, J=2.1 Hz, 1H), 6.69-6.63 (m, 2H), 6.11 (br s, 1H), 3.10 (s, 3H), 1.97-1.86 (m, 4H), 1.69-1.59 (m, 2H). 2H under DMSO.

### Example 7

### Example 7A 1″,5″-dimethyldispiro[1,3-dioxolane-2,1′-cyclohexane-4′,3″-indole]-2″-one

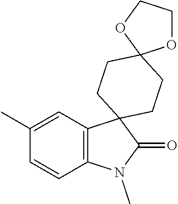 [SMILES: Cc1ccc2c(c1)C1(CCC3(CC1)OCCO3)C(=O)N2C]

To a microwave vial was added Example 6B (70 mg, 0.2 mmol, 1 eq), methyl boronic acid (24 mg, 0.4 mmol, 2 eq) and K₂CO₃ (82 mg, 0.6 mmol, 3 eq), followed by THE (2 mL) and water (0.2 mL). The mixture was sparged with N₂, followed by the addition of Pd(dppf)Cl₂ (15 mg, 0.02 mmol, 0.1 eq) and then heated at 120° C. for 1 h under microwave irradiation. The reaction was partitioned between EtOAc and water and the organic phase dried (MgSO₄) and concentrated in vacuo. Purification by flash chromatography (5 g silica cartridge) eluting with a stepped gradient of 0-30% EtOAc in heptane afforded Example 7A as a colourless gum (32 mg, 0.11 mmol, 56%). LRMS calculated for C₁₇H₂₁NO₃: 287; found: 288 (M+H). ¹H NMR (400 MHz, DMSO-d₆) δ ppm: 7.19 (app s, 1H), 7.11-7.08 (m, 1H), 6.90 (d, J=7.9 Hz, 1H), 3.98-3.90 (m, 4H), 3.10 (s, 3H), 2.30 (s, 3H), 2.16-2.06 (m, 2H), 1.84-1.67 (m, 6H).

### Example 7B 1′,5′-dimethylspiro[cyclohexane-1,3′-indole]-2′,4-dione

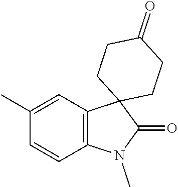 [SMILES: Cc1ccc2c(c1)C1(CCC(=O)CC1)C(=O)N2C]

A solution of Example 7A (32 mg, 0.11 mmol, 1 eq) in acetone (3 mL) was treated as described in General procedure 9 and the mixture heated at 50° C. for 1 h. The reaction was cooled to rt, diluted with water and then extracted into EtOAc, dried (MgSO₄) and concentrated in vacuo to afford Example 7B as a colourless solid (24 mg, 0.1 mmol, 88%) that was used directly in the subsequent step without further purification. LRMS calculated for C₁₅H₁₇NO₂: 243; found: 244 (M+H). ¹H NMR (400 MHz, DMSO-d₆) δ ppm: 7.37 (app s, 1H), 7.14-7.11 (m, 1H), 6.95 (d, J=7.9 Hz, 1H), 3.15 (s, 3H), 2.86 (ddd, J=15.5, 9.9, 5.9 Hz, 2H), 2.45 (dt, J=15.2, 5.9 Hz, 2H), 2.30 (s, 3H), 2.16-2.07 (m, 2H), 2.03-1.95 (m, 2H).

### Example 7 (1r,4r)-4-(3-bromoanilino)-1′,5′-dimethyl-2′-oxo-1′,2′-dihydrospiro[cyclohexane-1,3′-indole]-4-carboxylic acid

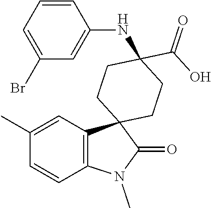 [SMILES: Cc1ccc2c(c1)[C@]1(CC[C@](Nc3cccc(Br)c3)(C(=O)O)CC1)C(=O)N2C]

A solution of Example 7B (24 mg, 0.1 mmol, 1 eq) in THE (2 mL) was treated as described in General Procedure 10 using 3-bromoaniline (11 μL, 0.1 mmol, 1 eq) and then stirred at rt for 18 h. The reaction was diluted with water, acidified with 2 M aq. HCl solution and extracted with DCM. The organic phase was loaded onto a DCM-wet PE-AX cartridge (5 g), washing successively with DCM, MeOH and then eluted with 5% HCOOH in DCM and concentrated in vacuo. Trituration with DCM afforded a single diastereoisomer, Example 7 as a white powder (3.2 mg, 0.01 mmol, 7%). Further purification of the washings by preparative HPLC at pH 9 afforded additional material, Example 7 as a cream solid (7.6 mg, 0.02 mmol, 17%). LRMS calculated for C₂₂H₂₃N₂O₃Br: 442; found: 443 (M+H). ¹H NMR (400 MHz, DMSO-d₆) δ ppm: 12.68 (br s, 1H), 7.12-7.06 (m, 2H), 7.03 (t, J=8.1 Hz, 1H), 6.88 (d, J=7.8 Hz, 1H), 6.85 (t, J=1.9 Hz, 1H), 6.71 (d, J=7.6 Hz, 1H), 6.64 (dd, J=9.5, 1.6 Hz, 1H), 6.28 (br s, 1H), 3.09 (s, 3H), 2.63-2.54 (m, 2H), 2.31 (s, 3H), 2.02-1.90 (m, 4H), 1.62-1.52 (m, 2H).

### Example 8

### Example 8A 5″-ethyl-1″-methyldispiro[1,3-dioxolane-2,1′-cyclohexane-4′,3″-indole]-2″-one

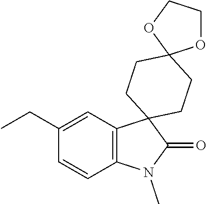 [SMILES: CCc1ccc2c(c1)C1(CCC3(CC1)OCCO3)C(=O)N2C]

To a microwave vial was added Example 6B (93 mg, 0.26 mmol, 1 eq), ethyl boronic acid (39 mg, 0.52 mmol, 2 eq) and K₂CO₃ (110 mg, 0.8 mmol, 3 eq), followed by THE (2 mL) and water (0.2 mL). The mixture was sparged with N₂, followed by the addition of Pd(dppf)Cl₂ (19 mg, 0.03 mmol, 0.1 eq) and then heated at 120° C. for 1 h under microwave irradiation. The reaction was partitioned between DCM and water, and the organic phase dried (MgSO₄) and concentrated in vacuo. Purification by flash chromatography (10 g silica cartridge) eluting with a stepped gradient of 0-20% EtOAc in heptane afforded Example 8A as a colourless gum (31 mg, 0.1 mmol, 39%). LRMS calculated for C₁₈H₂₃NO₃: 301; found: 302 (M+H). ¹H NMR (400 MHz, DMSO-d₆) δ ppm: 7.19 (d, J=1.7 Hz, 1H), 7.12 (dd, J=7.9, 1.7 Hz, 1H), 6.92 (d, J=7.9 Hz, 1H), 3.97-3.90 (m, 4H), 3.10 (s, 3H), 2.60 (q, J=7.6 Hz, 2H), 2.18-2.06 (m, 2H), 1.85-1.70 (m, 6H), 1.17 (t, J=7.6 Hz, 3H).

### Example 8B 5′-ethyl-1′-methylspiro[cyclohexane-1,3′-indole]-2′,4-dione

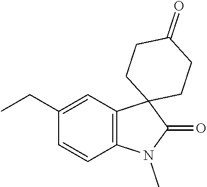 [SMILES: CCc1ccc2c(c1)C1(CCC(=O)CC1)C(=O)N2C]

A solution of Example 8A (31 mg, 0.1 mmol, 1 eq) in acetone (3 mL) was treated as described in General procedure 9 and stirred at 45° C. for 2 h. The reaction was allowed to cool to rt, diluted with water and extracted with EtOAc. The organic phase was dried (MgSO₄) and concentrated in vacuo to afford Example 8B as a colourless solid (21 mg, 0.08 mmol, 79%) that was used directly in the subsequent step without further purification. LRMS calculated for C₁₆H₁₉NO₂: 257; found: 258 (M+H). ¹H NMR (400 MHz, DMSO-d₆) δ ppm: 7.40 (d, J=1.7 Hz, 1H), 7.15 (dd, J=7.9, 1.7 Hz, 1H), 6.96 (d, J=7.9 Hz, 1H), 3.15 (s, 3H), 2.88 (ddd, J=15.6, 10.2, 5.7 Hz, 2H), 2.60 (q, J=7.6 Hz, 2H), 2.44 (dt, J=15.1, 5.7 Hz, 2H), 2.20-2.09 (m, 2H), 2.03-1.94 (m, 2H), 1.18 (t, J=7.6 Hz, 3H).

### Example 8 (1r,4r)-4-(3-bromoanilino)-5′-ethyl-1′-methyl-2′-oxo-1′,2′-dihydrospiro[cyclohexane-1,3′-indole]-4-carboxylic acid

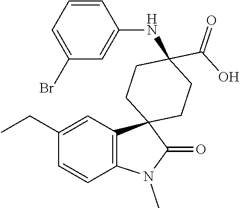 [SMILES: CCc1ccc2c(c1)[C@]1(CC[C@](Nc3cccc(Br)c3)(C(=O)O)CC1)C(=O)N2C]

A solution of Example 8B (21 mg, 0.08 mmol, 1 eq) in THE (2 mL) was treated as described in General procedure 10 using 3-bromoaniline (9 μL, 0.08 mmol, 1 eq) and then stirred at rt for 18 h. The reaction was diluted with water, acidified with 2 M aq. HCl solution and extracted with EtOAc. The organic phase was loaded onto a DCM-wet PE-AX cartridge (5 g), washing successively with DCM, MeOH and then eluted with 5% HCOOH in DCM and concentrated in vacuo. Further purification by preparative HPLC at pH 9 afforded a single diastereoisomer, Example 8 as a white powder (4.4 mg, 0.01 mmol, 11%). LRMS calculated for C₂₃H₂₅N₂O₃Br: 456; found: 457 (M+H). ¹H NMR (400 MHz, DMSO-d₆) δ ppm: 12.69 (br s, 1H), 7.15-7.08 (m, 2H), 7.03 (t, J=8.1 Hz, 1H), 6.90 (d, J=8.3 Hz, 1H), 6.86 (t, J=2.1 Hz, 1H), 6.70 (d, J=7.7 Hz, 1H), 6.65 (dd, J=8.3, 1.7 Hz, 1H), 6.27 (br s, 1H), 3.09 (s, 3H), 2.64-2.54 (m, 4H), 2.03-1.90 (m, 4H), 1.63-1.53 (m, 2H), 1.17 (t, J=7.6 Hz, 3H).

### Example 9

### Example 9A 1″-methyl-5″-[(E)-2-phenylethenyl]dispiro[1,3-dioxolane-2,1′-cyclohexane-4′,3″-indole]-2″-one

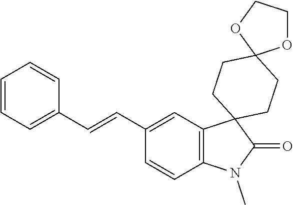 [SMILES: CN1C(=O)C2(CCC3(CC2)OCCO3)c2cc(/C=C/c3ccccc3)ccc21]

Example 6B (100 mg, 0.28 mmol, 1 eq), cis-B-styreneboronic acid (84 mg, 0.57 mmol, 2 eq) and K₂CO₃ (118 mg, 0.85 mmol, 3 eq) were suspended in THE (3 mL) and water (0.3 mL) and the mixture sparged with N₂. Pd(dppf)Cl₂ (21 mg, 0.03 mmol, 0.1 eq) was added and the reaction refluxed under a N₂ atmosphere for 5 h. The mixture was partitioned between DCM and water, and the organic phase dried (MgSO₄) and concentrated in vacuo. Purification by flash chromatography (10 g silica cartridge) eluting with a stepped gradient of 0-30% EtOAc in heptane afforded Example 9A as a white powder (106 mg, 0.28 mmol, 99%). LRMS calculated for C₂₄H₂₅NO₃: 375; found: 376 (M+H). ¹H NMR (400 MHz, DMSO-d₆) δ ppm: 7.64-7.58 (m, 3H), 7.54 (dd, J=8.2, 1.6 Hz, 1H), 7.37 (t, J=7.6 Hz, 2H), 7.31 (d, J=16.5 Hz, 1H), 7.27-7.18 (m, 2H), 7.04 (d, J=8.1 Hz, 1H), 4.00-3.92 (m, 4H), 3.15 (s, 3H), 2.24-2.13 (m, 2H), 1.95-1.73 (m, 6H).

### Example 9B 1′-methyl-5′-[(E)-2-phenylethenyl]spiro[cyclohexane-1,3′-indole]-2′,4-dione

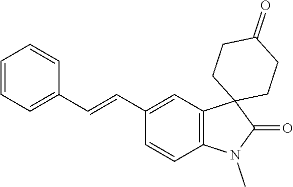 [SMILES: CN1C(=O)C2(CCC(=O)CC2)c2cc(/C=C/c3ccccc3)ccc21]

A solution of Example 9A (106 mg, 0.28 mmol, 1 eq) in acetone (3 mL) was treated as described in General procedure 9 and stirred at 45° C. for 3 h. The reaction was diluted with water, extracted with DCM and the organic phase dried (MgSO₄) and concentrated in vacuo to afford Example 9B as a colourless gum (64.4 mg, 0.19 mmol, 68%). LRMS calculated for C₂₂H₂₁NO₂: 331; found: 332 (M+H). ¹H NMR (400 MHz, DMSO-d₆) δ ppm: 7.90 (d, J=1.7 Hz, 1H), 7.59-7.55 (m, 2H), 7.53 (dd, J=8.1, 1.7 Hz, 1H), 7.38 (t, J=7.7 Hz, 2H), 7.28-7.22 (m, 3H), 7.08 (d, J=8.1 Hz, 1H), 3.20 (s, 3H), 2.89 (ddd, J=15.3, 9.8, 5.8 Hz, 2H), 2.21 (ddd, J=14.6, 9.9, 5.2 Hz, 2H), 2.05 (dt, J=13.0, 6.1 Hz, 2H). 2H under DMSO.

### Example 9 (1r,4r)-4-(3-bromoanilino)-1′-methyl-2′-oxo-5′-[(E)-2-phenylethenyl]-1′,2′-dihydrospiro[cyclohexane-1,3′-indole]-4-carboxylic acid

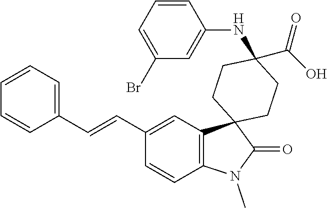 [SMILES: CN1c2ccc(/C=C/c3ccccc3)cc2[C@]2(CC[C@](Nc3cccc(Br)c3)(C(=O)O)CC2)C1=O]

A solution of Example 9B (64.4 mg, 0.19 mmol, 1 eq) in THE (3 mL) was treated as described in General procedure 10 using 3-bromoaniline (21 μL, 0.19 mmol, 1 eq) and then stirred at rt for 18 h. The reaction was diluted with water, acidified with 2 M aq. HCl solution and extracted with DCM. The organic phase was loaded onto a DCM-wet PE-AX cartridge (5 g), washing successively with DCM, MeOH and then eluted with 5% HCOOH in DCM and concentrated in vacuo. Trituration with Et₂O and then DCM afforded a single diastereoisomer, Example 9 as a white powder (6.9 mg, 0.01 mmol, 6%). LRMS calculated for C₂₉H₂₇N₂O₃Br: 530; found: 531 (M+H). ¹H NMR (400 MHz, DMSO-d₆) δ ppm: 12.71 (br s, 1H), 7.63-7.58 (m, 2H), 7.55 (dd, J=8.2, 1.6 Hz, 1H), 7.52 (d, J=1.7 Hz, 1H), 7.37 (t, J=7.7 Hz, 2H), 7.31 (d, J=16.4 Hz, 1H), 7.28-7.23 (m, 1H), 7.14 (d, J=16.4 Hz, 1H), 7.08-7.02 (m, 2H), 6.89 (t, J=2.1 Hz, 1H), 6.72 (dd, J=7.7, 1.8 Hz, 1H), 6.67 (dd, J=8.3, 1.8 Hz, 1H), 6.32 (br s, 1H), 3.14 (s, 3H), 2.66-2.58 (m, 2H), 2.13-1.98 (m, 4H), 1.66-1.56 (m, 2H).

### Example 10

### Example 10A 1″-methyl-5″-phenyldispiro[1,3-dioxolane-2,1′-cyclohexane-4′,3″-indole]-2″-one

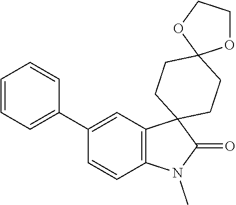 [SMILES: CN1C(=O)C2(CCC3(CC2)OCCO3)c2cc(-c3ccccc3)ccc21]

To a microwave vial was added Example 6B (80 mg, 0.23 mmol, 1 eq), phenylboronic acid (55 mg, 0.45 mmol, 2 eq) and K₂CO₃ (94 mg, 0.68 mmol, 3 eq) followed by THF (2 mL) and water (0.2 mL). The mixture was sparged with N₂, followed by the addition of Pd(dppf)Cl₂ (17 mg, 0.02 mmol, 0.1 eq) and then heated at 120° C. for 1 h under microwave irradiation. The reaction was partitioned between DCM and water and the organic phase dried (MgSO₄) and concentrated in vacuo. Purification by flash chromatography (10 g silica cartridge) eluting with a stepped gradient of 0-30% EtOAc in heptane afforded Example 10A as a cream powder (59 mg, 0.17 mmol, 74%) that was used directly in the subsequent step without further purification. LRMS calculated for C₂₂H₂₃NO₃: 349; found: 350 (M+H). ¹H NMR (400 MHz, DMSO-d₆) δ ppm: 7.69-7.63 (m, 2H), 7.60 (dd, J=8.1, 1.8 Hz, 1H), 7.57 (d, J=1.7 Hz, 1H), 7.48-7.42 (m, 2H), 7.36-7.31 (m, 1H), 7.12 (d, J=8.1 Hz, 1H), 3.97-3.93 (m, 4H), 3.17 (s, 3H), 2.24-2.13 (m, 2H), 1.96-1.74 (m, 6H).

### Example 10B 1′-methyl-5′-phenylspiro[cyclohexane-1,3′-indole]-2′,4-dione

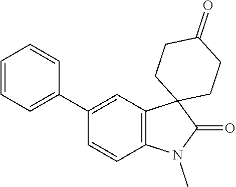 [SMILES: CN1C(=O)C2(CCC(=O)CC2)c2cc(-c3ccccc3)ccc21]

A solution of Example 10A (59 mg, 0.17 mmol, 1 eq) in acetone (3 mL) was treated as described in General procedure 9 and the mixture heated at 40° C. for 18 h. The reaction was cooled to rt and partitioned between DCM and water. The organic phase was dried (MgSO₄) and concentrated in vacuo to afford Example 10B as a cream oil (49 mg, 0.16 mmol, 95%) that was used directly in the subsequent step without further purification. LRMS calculated for C₂₀H₁₉NO₂: 305; found: 306 (M+H). ¹H NMR (400 MHz, DMSO-d₆) δ ppm: 7.85 (d, J=1.8 Hz, 1H), 7.71-7.66 (m, 2H), 7.63 (dd, J=8.1, 1.9 Hz, 1H), 7.48-7.42 (m, 2H), 7.36-7.31 (m, 1H), 7.15 (d, J=8.2 Hz, 1H), 3.22 (s, 3H), 2.99-2.87 (m, 2H), 2.49-2.41 (m, 2H), 2.35-2.24 (m, 2H), 2.07-1.99 (m, 2H).

### Example 10 (1r,4r)-4-(3-bromoanilino)-1′-methyl-2′-oxo-5′-phenyl-1′,2′-dihydrospiro[cyclohexane-1,3′-indole]-4-carboxylic acid

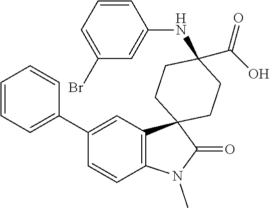 [SMILES: CN1c2ccc(-c3ccccc3)cc2[C@]2(CC[C@](Nc3cccc(Br)c3)(C(=O)O)CC2)C1=O]

A solution of Example 10B (49 mg, 0.16 mmol, 1 eq) in THE (3 mL) was treated as described in General procedure 10 using 3-bromoaniline (17 μL, 0.16 mmol, 1 eq) and then stirred at rt for 18 h. The reaction was diluted with water, acidified with 2 M aq. HCl solution and extracted with DCM. The organic phase was loaded onto a DCM-wet PE-AX cartridge (5 g), washing successively with DCM, MeOH and then eluted with 5% HCOOH in DCM and concentrated in vacuo. Purification by flash chromatography (5 g silica cartridge) eluting with a stepped gradient of 0-40% EtOAc in heptane followed by preparative HPLC at pH 9 afforded a single diastereoisomer, Example 10 as a yellow powder (4.5 mg, 0.01 mmol, 5%). LRMS calculated for C₂₇H₂₅N₂O₃Br: 504; found: 505 (M+H). ¹H NMR (400 MHz, DMSO-d₆) δ ppm: 7.64-7.58 (m, 2H), 7.58-7.53 (m, 2H), 7.50-7.43 (m, 2H), 7.37-7.30 (m, 1H), 7.10 (d, J=8.0 Hz, 1H), 6.99 (t, J=8.1 Hz, 1H), 6.89 (t, J=2.0 Hz, 1H), 6.70-6.63 (m, 2H), 6.15 (br s, 1H), 3.16 (s, 3H), 2.65-2.54 (m, 2H), 2.05-1.92 (m, 4H), 1.72-1.62 (m, 2H).

### Example 11

### Example 11A 1″-methyl-5″-(2-phenylethyl)dispiro[1,3-dioxolane-2,1′-cyclohexane-4′,3″-indole]-2″-one

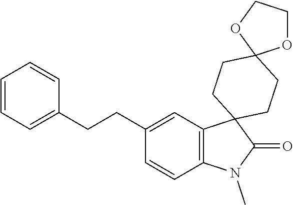 [SMILES: CN1C(=O)C2(CCC3(CC2)OCCO3)c2cc(CCc3ccccc3)ccc21]

To a partial solution of Example 9A (39 mg, 0.1 mmol, 1 eq) in EtOH (3 mL) and MeOH (3 mL) was added 10% Pd/C (catalytic) under a N₂ atmosphere. The mixture was evacuated and backfilled with N₂, then evacuated and backfilled with H₂ and shaken at rt under an atmosphere of H₂ for 2 h. The reaction was filtered through celite, eluted with MeOH and evaporated under reduced pressure to afford Example 11A as a grey solid (39 mg, 0.1 mmol, 99%) that was used directly in the subsequent step without further purification. LRMS calculated for C₂₄H₂₇NO₃: 377; found: 378 (M+H).

### Example 11B 1′-methyl-5′-(2-phenylethyl)spiro[cyclohexane-1,3′-indole]-2′,4-dione

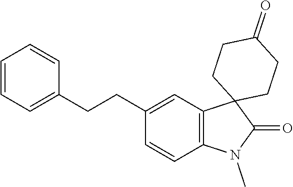 [SMILES: CN1C(=O)C2(CCC(=O)CC2)c2cc(CCc3ccccc3)ccc21]

A solution of Example 11A (39 mg, 0.1 mmol, 1 eq) in acetone (3 mL) was treated as described in General procedure 9 and stirred at 45° C. for 2 h. The reaction was diluted with water, extracted with DCM and the organic phase dried (MgSO₄) and concentrated in vacuo to afford Example 11B as a colourless oil (31 mg, 0.09 mmol, 89%) that was used directly in the subsequent step without further purification. LRMS calculated for C₂₂H₂₃NO₂: 333; found: 334 (M+H).

### Example 11 (1r,4r)-4-(3-bromoanilino)-1′-methyl-2′-oxo-5′-(2-phenylethyl)-1′,2′-dihydrospiro[cyclohexane-1,3′-indole]-4-carboxylic acid

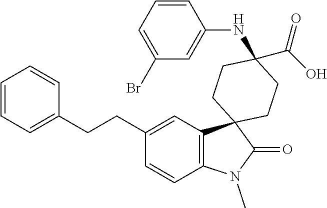 [SMILES: CN1c2ccc(CCc3ccccc3)cc2[C@]2(CC[C@](Nc3cccc(Br)c3)(C(=O)O)CC2)C1=O]

A solution of Example 11B (31 mg, 0.09 mmol, 1 eq) in THE (3 mL) was treated as described in General procedure 10 using 3-bromoaniline (10 μL, 0.09 mmol, 1 eq) and then stirred at rt for 18 h. The reaction was diluted with water, acidified with 2 M aq. HCl solution and extracted with DCM. The organic phase was loaded onto a DCM-wet PE-AX cartridge (5 g), washing successively with DCM, MeOH and then eluted with 5% HCOOH in DCM, and then further purified by preparative HPLC at pH 9. The material was partitioned between DCM and dilute aq. HCl solution, dried (MgSO₄) and concentrated in vacuo to afford a single diastereoisomer, Example 11 as a white powder (5.6 mg, 0.01 mmol, 11%). LRMS calculated for C₂₉H₂₉N₂O₃Br: 532; found: 533 (M+H). ¹H NMR (400 MHz, DMSO-d₆) δ ppm: 12.70 (br s, 1H), 7.29-7.18 (m, 4H), 7.17-7.00 (m, 4H), 6.93-6.84 (m, 2H), 6.72 (d, J=8.0 Hz, 1H), 6.64 (d, J=8.3 Hz, 1H), 6.26 (br s, 1H), 3.09 (s, 3H), 2.92-2.81 (m, 4H), 2.66-2.54 (m, 2H), 2.02-1.88 (m, 4H), 1.55 (app d, J=13.7 Hz, 2H).

### Example 12

### Example 12A 1″-methyl-5″-[(1E)-prop-1-en-1-yl]dispiro[1,3-dioxolane-2,1′-cyclohexane-4′,3″-indole]-2″-one

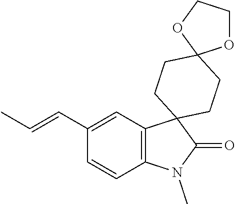 [SMILES: C/C=C/c1ccc2c(c1)C1(CCC3(CC1)OCCO3)C(=O)N2C]

Example 6B (100 mg, 0.28 mmol, 1 eq), trans-1-propen-1-ylboronic acid (49 mg, 0.57 mmol, 2 eq) and K₂CO₃ (118 mg, 0.85 mmol, 3 eq) were suspended in THE (3 mL) and water (0.3 mL) and the mixture sparged with N₂. Pd(dppf)Cl₂ (21 mg, 0.03 mmol, 0.1 eq) was added and the reaction refluxed under a N₂ atmosphere for 3 h. Additional trans-1-propen-1-ylboronic acid (49 mg, 0.57 mmol, 2 eq), K₂CO₃ (118 mg, 0.85 mmol, 3 eq) and Pd(dppf)Cl₂ (21 mg, 0.03 mmol, 0.1 eq) were added and the mixture refluxed under a N₂ atmosphere for 18 h. The mixture was partitioned between DCM and water, and the organic phase dried (MgSO₄) and concentrated in vacuo. Purification by flash chromatography (10 g silica cartridge) eluting with a stepped gradient of 0-20% EtOAc in heptane afforded Example 12A as a colourless gum (68.8 mg, 0.22 mmol, 77%). LRMS calculated for C₁₉H₂₃NO₃: 313; found: 314 (M+H). ¹H NMR (400 MHz, DMSO-d₆) δ ppm: 7.36 (d, J=1.6 Hz, 1H), 7.28 (dd, J=8.1, 1.6 Hz, 1H), 6.95 (d, J=8.1 Hz, 1H), 6.42 (dq, J=15.8, 1.8 Hz, 1H), 6.21 (dq, J=15.8, 6.5 Hz, 1H), 3.98-3.89 (m, 4H), 3.11 (s, 3H), 2.19-2.08 (m, 2H), 1.85-1.70 (m, 9H).

### Example 12B 1′-methyl-5′-[(1E)-prop-1-en-1-yl]spiro[cyclohexane-1,3′-indole]-2′,4-dione

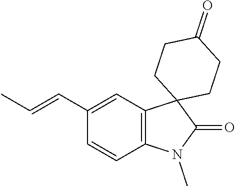 [SMILES: C/C=C/c1ccc2c(c1)C1(CCC(=O)CC1)C(=O)N2C]

A solution of Example 12A (68.8 mg, 0.22 mmol, 1 eq) in acetone (3 mL) was treated as described in General procedure 9 and stirred at 45° C. for 2 h. The reaction was diluted with water, extracted with DCM and the organic phase dried (MgSO₄) and concentrated in vacuo to afford Example 12B as a cream solid (55 mg, 0.2 mmol, 93%) that was used directly in the subsequent step without further purification. LRMS calculated for C₁₇H₁₉NO₂: 269; found: 270 (M+H). ¹H NMR (400 MHz, DMSO-d₆) δ ppm: 7.61 (d, J=1.7 Hz, 1H), 7.27 (dd, J=8.1, 1.7 Hz, 1H), 6.98 (d, J=8.0 Hz, 1H), 6.38 (dq, J=15.8, 1.7 Hz, 1H), 6.25 (dq, J=15.8, 6.4 Hz, 1H), 3.15 (s, 3H), 2.92-2.81 (m, 2H), 2.48-2.39 (m, 2H), 2.20-2.09 (m, 2H), 2.04-1.94 (m, 2H), 1.82 (dd, J=6.5, 1.5 Hz, 3H).

### Example 12 (1r,4r)-4-(3-bromoanilino)-1′-methyl-2′-oxo-5′-[(1E)-prop-1-en-1-yl]-1′,2′-dihydrospiro[cyclohexane-1,3′-indole]-4-carboxylic acid

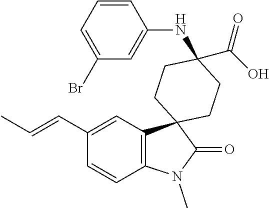 [SMILES: C/C=C/c1ccc2c(c1)[C@]1(CC[C@](Nc3cccc(Br)c3)(C(=O)O)CC1)C(=O)N2C]

A solution of Example 12B (55 mg, 0.2 mmol, 1 eq) in THF (3 mL) was treated as described in General procedure 10 using 3-bromoaniline (22 μL, 0.2 mmol, 1 eq) and then stirred at rt for 18 h. The reaction was diluted with water, acidified with 2 M aq. HCl solution and extracted with DCM. The organic phase was loaded onto a DCM-wet PE-AX cartridge (5 g), washing successively with DCM, MeOH and then eluted with 5% HCOOH in DCM and concentrated in vacuo. Purification by flash chromatography (10 g silica cartridge) eluting with a stepped gradient of 0-100% EtOAc in heptane was followed by preparative HPLC at pH 9. The material was dissolved in DCM and washed with dilute aq. HCl solution, dried (MgSO₄) and concentrated in vacuo to afford a single diastereoisomer, Example 12 as a white powder (4.1 mg, 0.01 mmol, 4%). LRMS calculated for C₂₄H₂₅N₂O₃Br: 468; found: 469 (M+H). ¹H NMR (399 MHz, DMSO-d6) δ ppm: 12.69 (br s, 1H), 7.30-7.24 (m, 2H), 7.03 (t, J=8.1 Hz, 1H), 6.93 (d, J=8.0 Hz, 1H), 6.85 (t, J=2.1 Hz, 1H), 6.70 (d, J=8.0 Hz, 1H), 6.66-6.62 (m, 1H), 6.42 (dq, J=15.9, 1.6 Hz, 1H), 6.26 (br s, 1H), 6.18 (dq, J=15.9, 6.7 Hz, 1H), 3.10 (s, 3H), 2.64-2.54 (m, 2H), 2.04-1.92 (m, 4H), 1.83 (dd, J=6.7, 1.6 Hz, 3H), 1.63-1.53 (m, 2H).

### Example 13 (1r,4r)-1′-benzyl-4-(3-bromoanilino)-2′-oxo-1′,2′-dihydrospiro[cyclohexane-1,3′-indole]-4-carboxylic acid

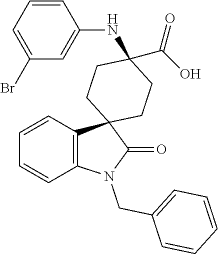 [SMILES: O=C(O)[C@]1(Nc2cccc(Br)c2)CC[C@@]2(CC1)C(=O)N(Cc1ccccc1)c1ccccc12]

To a solution of Example 1 (100 mg, 0.24 mmol, 1 eq) in DMF, cooled in an ice bath under a N₂ atmosphere, was added NaH (60% dispersion; 21 mg, 0.53 mmol, 2.2 eq) and the mixture stirred for 10 min. BnBr (72 μL, 0.6 mmol, 2.5 eq) was added and the reaction stirred at rt for 18 h. A further portion of BnBr (72 μL, 0.6 mmol, 2.5 eq) was added and the mixture stirred at rt for 48 h. The reaction was treated with a further portion of NaH (60% dispersion; 21 mg, 0.53 mmol, 2.2 eq) and stirred at rt for 2 h. The mixture was diluted with water and acidified with 2 M aq. HCl solution. The precipitate was collected by filtration and washed with water. Further purification by flash chromatography (5 g silica cartridge) eluting with a stepped gradient of 0-40% EtOAc in heptane afforded Example 13 as a white powder (30 mg, 0.06 mmol, 24%). LRMS calculated for C₂₇H₂₅N₂O₃Br: 504; found: 505 (M+H). ¹H NMR (400 MHz, DMSO-d₆) δ ppm: 12.73 (br s, 1H), 7.36-7.22 (m, 6H), 7.17 (td, J=7.7, 1.2 Hz, 1H), 7.03 (app t, J=8.0 Hz, 2H), 6.89-6.84 (m, 2H), 6.72-6.68 (m, 1H), 6.65-6.61 (m, 1H), 6.32 (br s, 1H), 4.88 (s, 2H), 2.70-2.56 (m, 2H), 2.10-1.96 (m, 4H), 1.71-1.60 (m, 2H).

### Example 14

### Example 14A 10″-bromodispiro[1,3-dioxolane-2,1′-cyclohexane-4′,2″-[4]azatricyclo[6.3.1.0\hat{ }{4,12}]dodecane]-1″(11″),8″(12″),9″-trien-3″-one

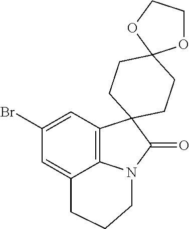 [SMILES: O=C1N2CCCc3cc(Br)cc(c32)C12CCC1(CC2)OCCO1]

6-Bromo-1-azatricyclo[6.3.1.0\hat{ }{4,12}]dodeca-4 (12),5,7-trien-2-one (50 mg, 0.2 mmol, 1 eq) and Preparation 1b (57 mg, 0.2 mmol, 1 eq) were treated as described according to General procedure 8a. The reaction was quenched with sat. aq. NH₄Cl solution, diluted with sat. aq. NaHCO₃ solution and then extracted with EtOAc. The organic phase was washed with brine, dried (MgSO₄) and concentrated in vacuo. Purification by flash chromatography (10 g silica cartridge) eluting with a stepped gradient of 0-30% EtOAc in heptane afforded Example 14A as a cream powder (35.3 mg, 0.09 mmol, 47%). LRMS calculated for C₁₈H₂₀NO₃Br: 377; found: 378 (M+H). ¹H NMR (400 MHz, DMSO-d₆) δ ppm: 7.34-7.30 (m, 2H), 3.96-3.90 (m, 4H), 3.62-3.55 (m, 2H), 2.74 (t, J=6.0 Hz, 2H), 2.10-2.01 (m, 2H), 1.94-1.82 (m, 4H), 1.79-1.67 (m, 4H).

### Example 14B 10′-bromo-4′-azaspiro[cyclohexane-1,2′-tricyclo[6.3.1.0\hat{ }{4,12}]dodecane]-1′(11′),8′(12′),9′-triene-3′,4-dione

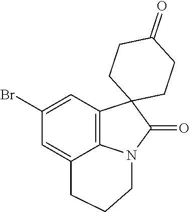 [SMILES: O=C1CCC2(CC1)C(=O)N1CCCc3cc(Br)cc2c31]

A solution of Example 14A (35 mg, 0.09 mmol, 1 eq) in acetone (3 mL) was treated as described according to General procedure 9 and stirred at 45° C. for 2 h. The mixture was diluted with water, extracted with DCM and the combined organic extracts dried (MgSO₄) and concentrated in vacuo to afford Example 14B as a cream powder (28.5 mg, 0.09 mmol, 92%). LRMS calculated for C₁₆H₁₆NO₂Br: 333; found: 334 (M+H). ¹H NMR (400 MHz, DMSO-d₆) δ ppm: 7.61 (d, J=1.8 Hz, 1H), 7.32 (d, J=1.7 Hz, 1H), 3.65-3.60 (m, 2H), 2.85-2.72 (m, 4H), 2.49-2.41 (m, 2H), 2.20-2.10 (m, 2H), 2.06-1.97 (m, 2H), 1.97-1.89 (m, 2H).

### Example 14 8′-bromo-4-(3-bromoanilino)-2′-oxo-5′,6′-dihydro-2′H,4′H-spiro[cyclohexane-1,1′-pyrrolo[3,2,1-ij]quinoline]-4-carboxylic acid, diastereoisomer 1

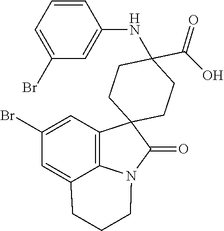 [SMILES: O=C(O)C1(Nc2cccc(Br)c2)CCC2(CC1)C(=O)N1CCCc3cc(Br)cc2c31]

A solution of Example 14B (28.5 mg, 0.09 mmol, 1 eq) in THF (3 mL) was treated as described according to General procedure 10 using 3-bromoaniline (9 μL, 0.09 mmol, 1 eq) and the mixture stirred at rt for 48 h. The reaction was diluted with water, acidified with 2 M aq. HCl solution and extracted with DCM. The organic extracts were loaded onto a DCM-wet PE-AX cartridge (5 g), washed successively with DCM, MeOH and eluted with 5% HCOOH in DCM, and concentrated in vacuo. Purification by flash chromatography (5 g silica cartridge) eluting with a stepped gradient of 0-2% MeOH in DCM was followed by preparative HPLC at pH 9.

The resulting material was dissolved in DCM, washed with dilute aq. HCl solution, and the organic phase dried (MgSO₄) and concentrated in vacuo to afford a single diastereoisomer, Example 14 as a white powder (4.7 mg, 0.01 mmol, 10%). LRMS calculated for C₂₃H₂₂N₂O₃Br₂: 532; found: 533 (M+H). 1H NMR (400 MHz, DMSO-d6) δ12.75 (br s, 1H), 7.33-7.27 (m, 2H), 7.03 (t, J=8.0 Hz, 1H), 6.85 (s, 1H), 6.74-6.69 (m, 1H), 6.67-6.62 (m, 1H), 6.21 (br s, 1H), 3.61-3.53 (m, 2H), 2.76-2.70 (m, 2H), 2.00-1.82 (m, 6H), 1.73-1.62 (m, 2H). 2H under DMSO.

### Example 15

### Example 15A 5″-cyclopropyl-1″-methyldispiro[1,3-dioxolane-2,1′-cyclohexane-4′,3″-indole]-2″-one

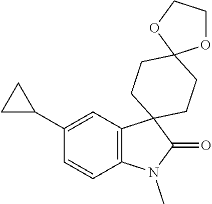 [SMILES: CN1C(=O)C2(CCC3(CC2)OCCO3)c2cc(C3CC3)ccc21]

To a microwave vial was added Example 6B (87 mg, 0.25 mmol, 1 eq), cyclopropylboronic acid (32 mg, 0.37 mmol, 1.5 eq) and K₂CO₃ (102 mg, 0.74 mmol, 3 eq) followed by THE (2 mL) and water (0.2 mL). The mixture was sparged with N₂, followed by the addition of Pd(dppf)Cl₂ (18 mg, 0.02 mmol, 0.1 eq) and heated at 120° C. for 1 h under microwave irradiation. Additional cyclopropylboronic acid (32 mg, 0.37 mmol, 1.5 eq), K₂CO₃ (102 mg, 0.74 mmol, 3 eq) and Pd(dppf)Cl₂ (18 mg, 0.02 mmol, 0.1 eq) were added and the mixture heated at 120° C. for 1 h under microwave irradiation. The reaction was partitioned between DCM and water, and the organic phase washed with brine, dried (MgSO₄) and concentrated in vacuo. Purification by flash chromatography (10 g silica cartridge) eluting with a stepped gradient of 0-30% EtOAc in heptane afforded Example 15A as a yellow oil (37 mg, 0.12 mmol, 47%) that was used directly in the subsequent step without further purification. LRMS calculated for C₁₉H₂₃NO₃: 313; found: 314 (M+H). ¹H NMR (400 MHz, DMSO-d₆) δ ppm: 7.05 (d, J=1.8 Hz, 1H), 6.98 (dd, J=8.1, 1.8 Hz, 1H), 6.89 (d, J=8.0 Hz, 1H), 3.98-3.90 (m, 4H), 3.09 (s, 3H), 2.18-2.09 (m, 2H), 1.98-1.89 (m, 1H), 1.80-1.68 (m, 6H), 0.93-0.88 (m, 2H), 0.64-0.60 (m, 2H).

### Example 15B 5′-cyclopropyl-1′-methylspiro[cyclohexane-1,3′-indole]-2′,4-dione

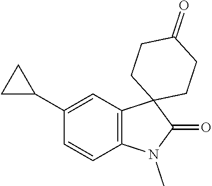 [SMILES: CN1C(=O)C2(CCC(=O)CC2)c2cc(C3CC3)ccc21]

A solution of Example 15A (37 mg, 0.12 mmol, 1 eq) in acetone (3 mL) was treated as described in General procedure 9 and the mixture heated at 45° C. for 1 h. The reaction was cooled to rt, diluted with water and extracted with DCM. The organic phase was dried (MgSO₄) and concentrated in vacuo to afford Example 15B as a yellow oil (30 mg, 0.11 mmol, 94%) that was used directly in the subsequent step without further purification. LRMS calculated for C₁₇H₁₉NO₂: 269; found: 270 (M+H).

### Example 15 (1r,4r)-4-(3-bromoanilino)-5′-cyclopropyl-1′-methyl-2′-oxo-1′,2′-dihydrospiro[cyclohexane-1,3′-indole]-4-carboxylic acid

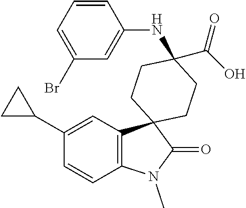 [SMILES: CN1c2ccc(C3CC3)cc2[C@]2(CC[C@](Nc3cccc(Br)c3)(C(=O)O)CC2)C1=O]

A solution of Example 15B (30 mg, 0.11 mmol, 1 eq) in THE (3 mL) was treated as described in General procedure 10 using 3-bromoaniline (12 μL, 0.11 mmol, 1 eq) and then stirred at rt for 48 h. The reaction was diluted with water, acidified with 2 M aq. HCl solution and extracted with DCM. The organic phase was loaded onto a DCM-wet PE-AX cartridge (5 g), washing successively with DCM, MeOH and then eluted with 5% HCOOH in DCM and concentrated in vacuo. Purification by flash chromatography (5 g silica cartridge) eluting with a stepped gradient of 0-50% EtOAc in heptane and then 10% MeOH in DCM was followed by preparative HPLC at pH 9. The material was dissolved in DCM, washed with dilute aq. HCl solution and the organic phase dried (MgSO₄) and concentrated in vacuo to afford a single diastereoisomer, Example 15 as a white powder (3.5 mg, 0.01 mmol, 6%). LRMS calculated for C₂₄H₂₅N₂O₃Br: 468; found: 469 (M+H). ¹H NMR (400 MHz, DMSO-d₆) δ ppm: 12.72 (br s, 1H), 7.01-6.99 (m, 2H), 6.96 (dd, J=8.0, 1.8 Hz, 1H), 6.89-6.85 (m, 2H), 6.73-6.68 (m, 1H), 6.68-6.63 (m, 1H), 6.25 (br s, 1H), 3.08 (s, 3H), 2.64-2.54 (m, 2H), 2.03-1.89 (m, 5H), 1.62-1.51 (m, 2H), 0.94-0.86 (m, 2H), 0.63-0.57 (m, 2H).

### Example 17

### Example 17A (1s,4s)-4-(3-bromoanilino)-2′-oxo-1′,2′-dihydrospiro[cyclohexane-1,3′-indole]-4-carbonitrile

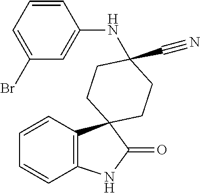 [SMILES: N#C[C@]1(Nc2cccc(Br)c2)CC[C@]2(CC1)C(=O)Nc1ccccc12]

A stirred solution of 1′H-spiro[cyclohexane-1,3′-indole]-2′,4-dione, (185 mg, 0.86 mmol, 1 eq) was treated as described in General procedure 11 using 3-bromoaniline (94 μL, 0.86 mmol, 1 eq) in AcOH (2 mL). TMS-CN (112.9 μL, 0.9 mmol, 1.05 eq) was added and the reaction stirred at rt for 18 h. The mixture was added to ice-cold 7 M aq. NH₃ solution (25 mL) and extracted with EtOAc. The organic phase was washed with water, dried (MgSO₄) and concentrated in vacuo to afford a single diastereoisomer, Example 17A as a solid (347 mg, 0.88 mmol, quant.). LRMS calculated for C₂₀H₁₈N₃OBr: 395; found: 396 (M+H). ¹H NMR (400 MHz, DMSO-d₆) δ ppm: 10.44 (s, 1H), 7.26 (d, J=7.4 Hz, 1H), 7.22 (td, J=7.7, 1.2 Hz, 1H), 7.16 (t, J=8.1 Hz, 1H), 7.06 (t, J=2.1 Hz, 1H), 7.02 (td, J=7.6, 1.1 Hz, 1H), 6.93-6.88 (m, 2H), 6.87 (d, J=7.6 Hz, 1H), 6.58 (s, 1H), 2.49-2.42 (m, 2H), 2.38-2.28 (m, 2H), 1.95-1.86 (m, 4H).

### Example 17B (1s,4s)-4-(3-bromoanilino)-2′-oxo-1′,2′-dihydrospiro[cyclohexane-1,3′-indole]-4-carboxamide

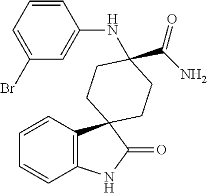 [SMILES: NC(=O)[C@]1(Nc2cccc(Br)c2)CC[C@]2(CC1)C(=O)Nc1ccccc12]

A solution of Example 17A (347 mg, 0.88 mmol, 1 eq) in MeOH (25 mL), cooled to 0° C., was treated as described in General procedure 12b, using 1 M aq. NaOH solution (4.38 mL, 4.38 mmol, 5 eq), followed by the dropwise addition of H₂O₂ (30 wt % solution in water; 2.58 mL, 10.2 M, 0.03 mol, 30 eq) and the mixture heated at 35° C. for 18 h and then at rt for 48 h. The mixture was cooled in an ice-water bath and acidified by the slow addition of 2 M aq. HCl solution. The MeOH was removed in vacuo and the residue partitioned between EtOAc and water. The organic phase was washed with brine, dried (MgSO₄) and concentrated in vacuo.

Purification by automated flash chromatography (CombiFlash Rf, 40 g RediSep™ silica cartridge) eluting with a gradient of 0-95% EtOAc in heptane afforded Example 17B as a white solid (171.4 mg, 0.41 mmol, 47%). LRMS calculated for C₂₀H₂₀N₃O₂Br: 413; found: 414 (M+H). ¹H NMR (400 MHz, DMSO-d₆) δ ppm: 10.38 (s, 1H), 7.45 (d, J=7.4 Hz, 1H), 7.36 (br s, 1H), 7.24-7.17 (m, 2H), 7.04 (t, J=8.1 Hz, 1H), 6.99 (td, J=7.6, 1.2 Hz, 1H), 6.90-6.86 (m, 2H), 6.73 (ddd, J=7.8, 1.9, 0.8 Hz, 1H), 6.62 (ddd, J=8.2, 2.4, 0.9 Hz, 1H), 6.18 (s, 1H), 2.35-2.25 (m, 2H), 2.12-1.93 (m, 4H), 1.42-1.34 (m, 2H).

### Example 17C (1s,4s)-4-(3-bromoanilino)-2′-oxo-1′,2′-dihydrospiro[cyclohexane-1,3′-indole]-4-carboxylic acid

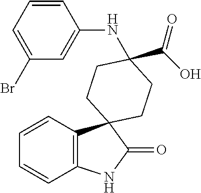 [SMILES: O=C(O)[C@]1(Nc2cccc(Br)c2)CC[C@]2(CC1)C(=O)Nc1ccccc12]

A solution of Example 17B (150 mg, 0.36 mmol, 1 eq) in 2-methoxyethanol (2 mL) was treated as described in General procedure 13, using 6 M NaOH solution (1.5 mL, 8.98 mmol, 24.8 eq) and the mixture heated at 200° C. for 30 min under microwave irradiation. The reaction was diluted with water, acidified by the addition of 2 M aq. HCl solution and extracted with EtOAc. The combined organic extracts were washed with brine, dried (MgSO₄) and concentrated in vacuo. The residue was dissolved in minimal MeOH with the addition of a few drops of DIPEA, loaded onto a MeOH-wet PE-AX cartridge (10 g) and washed successively with MeOH, DCM and eluted with 10% HCOOH in DCM, and then concentrated in vacuo to afford Example 17C as a beige solid (94 mg, 0.23 mmol, 62%). LRMS calculated for C₂₀H₁₉N₂O₃Br: 414; found: 415 (M+H). ¹H NMR (400 MHz, DMSO-d₆) δ ppm: 12.78 (br s, 1H), 10.41 (s, 1H), 7.40 (d, J=7.5 Hz, 1H), 7.22 (td, J=7.7, 1.1 Hz, 1H), 7.06-6.98 (m, 2H), 6.89 (dd, J=7.7, 1.0 Hz, 1H), 6.80 (t, J=2.1 Hz, 1H), 6.72-6.68 (m, 1H), 6.62-6.58 (m, 1H), 6.36 (br s, 1H), 2.31-2.16 (m, 4H), 2.07-1.95 (m, 2H), 1.48-1.38 (m, 2H).

### Example 17D methyl (1s,4s)-4-(3-bromoanilino)-1′-methyl-2′-oxo-1′,2′-dihydrospiro[cyclohexane-1,3′-indole]-4-carboxylate

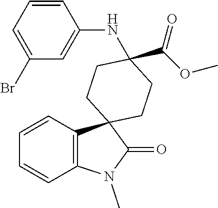 [SMILES: COC(=O)[C@]1(Nc2cccc(Br)c2)CC[C@]2(CC1)C(=O)N(C)c1ccccc12]

To a solution of Example 17C (80 mg, 0.19 mmol, 1 eq) in MeCN (10 mL) was added Cs₂CO₃ (188 mg, 0.58 mmol, 3 eq) and MeI (0.04 mL, 0.58 mmol, 3 eq) and the mixture stirred at rt for 18 h. The reaction was partitioned between DCM and water and the organic phase dried (MgSO₄) and concentrated in vacuo to afford Example 17D as a white solid (81 mg, 0.18 mmol, 94%). LRMS calculated for C₂₂H₂₃N₂O₃Br: 442; found: 443 (M+H). ¹H NMR (400 MHz, DMSO-d₆) δ ppm: 7.45 (d, J=7.4 Hz, 1H), 7.33 (t, 1H), 7.12-7.00 (m, 3H), 6.78 (t, J=2.1 Hz, 1H), 6.71 (dd, J=7.7, 1.8 Hz, 1H), 6.51 (dd, J=8.2, 2.2 Hz, 1H), 6.47 (s, 1H), 3.68 (s, 3H), 3.13 (s, 3H), 2.35-2.19 (m, 4H), 2.08-1.96 (m, 2H), 1.49-1.39 (m, 2H).

### Example 17 (1s,4s)-4-(3-bromoanilino)-1′-methyl-2′-oxo-1′,2′-dihydrospiro[cyclohexane-1,3′-indole]-4-carboxylic acid

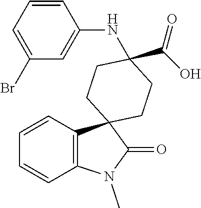 [SMILES: CN1c2ccccc2[C@]2(CC[C@@](Nc3cccc(Br)c3)(C(=O)O)CC2)C1=O]

To a solution of Example 17D (81 mg, 0.18 mmol, 1 eq) in MeOH (4 mL) was added 1 M aq. NaOH solution (0.91 mL, 0.91 mmol, 5 eq) and the mixture heated at 120° C. for 30 min under microwave irradiation. The reaction was concentrated in vacuo, dissolved in minimal water and acidified to pH 3 by the addition of 2 M aq. HCl solution. The aq. phase was extracted with DCM and the organic phase dried (PTFE phase separator) and concentrated in vacuo to afford Example 17 as a solid (76 mg, 0.18 mmol, 96%). LRMS calculated for C₂₁H₂₁N₂O₃Br: 428; found: 429 (M+H). ¹H NMR (400 MHz, DMSO-d₆) δ ppm: 12.88 (br s, 1H), 7.45 (d, J=7.3 Hz, 1H), 7.34 (td, J=7.7, 1.1 Hz, 1H), 7.13-7.00 (m, 3H), 6.80 (t, J=2.0 Hz, 1H), 6.70 (ddd, J=7.8, 1.9, 0.8 Hz, 1H), 6.61 (ddd, J=8.3, 2.3, 0.9 Hz, 1H), 6.39 (br s, 1H), 3.14 (s, 3H), 2.32-2.17 (m, 4H), 2.09-1.97 (m, 2H), 1.49-1.38 (m, 2H).

### Example 18

### Example 18A methyl (1r,4r)-4-(3-bromoanilino)-5′-iodo-1′-methyl-2′-oxo-1′,2′-dihydrospiro[cyclohexane-1,3′-indole]-4-carboxylate

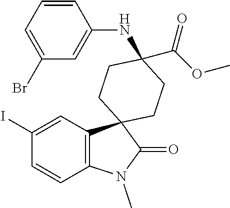 [SMILES: COC(=O)[C@]1(Nc2cccc(Br)c2)CC[C@@]2(CC1)C(=O)N(C)c1ccc(I)cc12]

To a solution of Example 5 (269 mg, 0.48 mmol, 1 eq) in DMF (5 mL), cooled in an ice bath under a N₂ atmosphere, was added NaH (60% dispersion; 21 mg, 0.53 mmol, 1.1 eq) and the mixture stirred for 5 min. MeI (45 μL, 0.73 mmol, 1.5 eq) was added and the reaction was slowly allowed to warm to rt and stirred for 5 h. The mixture was diluted with water, stirred for 5 min and the resulting precipitate collected by filtration. The solid was dissolved in DCM, washed with water and then dried (MgSO₄) and concentrated in vacuo. Purification by flash chromatography (10 g silica cartridge) eluting with a stepped gradient of 0-30% EtOAc in heptane afforded Example 18A as a cream powder (79 mg, 0.14 mmol, 28%). LRMS calculated for C₂₂H₂₂N₂O₃BrI: 568; found: 569 (M+H). ¹H NMR (400 MHz, DMSO-d₆) δ ppm: 7.63 (dd, J=8.1, 1.7 Hz, 1H), 7.57 (d, J=1.7 Hz, 1H), 7.04 (t, J=8.1 Hz, 1H), 6.88 (d, J=8.2 Hz, 1H), 6.83 (t, J=2.1 Hz, 1H), 6.75-6.71 (m, 1H), 6.54-6.50 (m, 1H), 6.32 (s, 1H), 3.65 (s, 3H), 3.09 (s, 3H), 2.64-2.54 (m, 2H), 2.08-1.93 (m, 4H), 1.62-1.52 (m, 2H).

### Example 18B methyl (1r,4r)-4-(3-bromoanilino)-5′-{[tert-butyl(dimethyl)silyl]ethynyl}-1′-methyl-2′-oxo-1′,2′-dihydrospiro[cyclohexane-1,3′-indole]-4-carboxylate

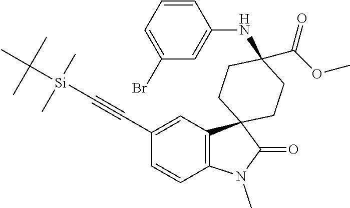 [SMILES: COC(=O)[C@]1(Nc2cccc(Br)c2)CC[C@@]2(CC1)C(=O)N(C)c1ccc(C#C[Si](C)(C)C(C)(C)C)cc12]

To a microwave vial was added Example 18A (40 mg, 0.07 mmol, 1 eq), 2-((tert-butyldimethylsilanyl)ethynyl) boronic acid pinacol ester (28 mg, 0.11 mmol, 1.5 eq) and K₂CO₃ (29 mg, 0.21 mmol, 3 eq) followed by THE (2 mL) and water (0.2 mL). The mixture was sparged with N₂, followed by the addition of Pd(dppf)Cl₂ (5 mg, 0.01 mmol, 0.1 eq) and then heated at 120° C. for 1 h under microwave irradiation. The reaction was partitioned between DCM and water, and the organic phase washed with brine, dried (MgSO₄) and concentrated in vacuo. Purification by flash chromatography (5 g silica cartridge) eluting with a stepped gradient of 0-30% EtOAc in heptane afforded Example 18B as a yellow gum (19.6 mg, 0.03 mmol, 47%) that was used directly in the subsequent step without further purification. LRMS calculated for C₃₀H₃₇N₂O₃SiBr: 580; found: 581 (M+H).

### Example 18 (1r,4r)-4-(3-bromoanilino)-5′-ethynyl-1′-methyl-2′-oxo-1′,2′-dihydrospiro[cyclohexane-1,3′-indole]-4-carboxylic acid

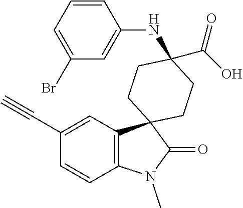 [SMILES: C#Cc1ccc2c(c1)[C@]1(CC[C@](Nc3cccc(Br)c3)(C(=O)O)CC1)C(=O)N2C]

To a solution of Example 18B (19.6 mg, 0.03 mmol, 1 eq) in MeOH (2 mL) was added 2 M aq. NaOH solution (0.5 mL) and the reaction stirred at 50° C. for 3 h. The reaction was cooled to rt, diluted with water and acidified with 2 M aq HCl solution. The mixture was extracted with DCM and the organic phase was loaded onto a DCM-wet PE-AX cartridge (5 g) and washed successively with DCM, MeOH and eluted with 5% HCOOH in DCM and concentrated in vacuo. Further purification by flash chromatography (5 g silica cartridge) eluting with a stepped gradient of 0-2% MeOH in DCM followed by preparative HPLC at pH 9 and subsequently at pH 4 afforded Example 18 as a white powder (2.5 mg, 0.01 mmol, 16%). LRMS calculated for C₂₃H₂₁N₂O₃Br: 452; found: 453 (M+H). ¹H NMR (400 MHz, DMSO-d₆) δ ppm: 7.44-7.40 (m, 2H), 7.04-6.98 (m, 2H), 6.87 (t, J=2.1 Hz, 1H), 6.71-6.64 (m, 2H), 6.17 (br s, 1H), 4.11 (s, 1H), 3.12 (s, 3H), 1.99-1.86 (m, 4H), 1.69-1.56 (m, 2H). 2H under DMSO.

### Example 19

### Example 19A methyl (1r,4r)-4-(3-bromoanilino)-1′-methyl-2′-oxo-5′-[(1E)-3-phenylprop-1-en-1-yl]-1′,2′-dihydrospiro[cyclohexane-1,3′-indole]-4-carboxylate

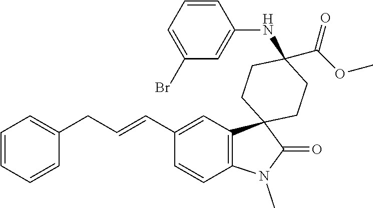 [SMILES: COC(=O)[C@]1(Nc2cccc(Br)c2)CC[C@@]2(CC1)C(=O)N(C)c1ccc(/C=C/Cc3ccccc3)cc12]

To a microwave vial was added Example 18A (33 mg, 0.06 mmol, 1 eq) trans-3-phenylpropen-1-yl-boronic acid (14 mg, 0.09 mmol, 1.5 eq.) and K₂CO₃ (24 mg, 0.17 mmol, 3 eq) followed by THF (1 mL) and water (0.1 mL). The mixture was sparged with N₂, followed by the addition of Pd(dppf)Cl₂ (4 mg, 0.01 mmol, 0.1 eq) and then heated at 120° C. for 1 h under microwave irradiation. The reaction was partitioned between DCM and water and the organic phase was washed with brine, dried (MgSO₄) and concentrated in vacuo. Purification by flash chromatography (5 g silica cartridge) eluting with a stepped gradient of 0-50% EtOAc in heptane, followed by trituration with heptane, afforded Example 19A as a cream powder (32 mg, 0.06 mmol, 98%) that was used directly in the subsequent step without further purification. LRMS calculated for C₃₁H₃₁N₂O₃Br: 558; found: 559 (M+H).

### Example 19 (1r,4r)-4-(3-bromoanilino)-1′-methyl-2′-oxo-5′-[(1E)-3-phenylprop-1-en-1-yl]-1′,2′-dihydrospiro[cyclohexane-1,3′-indole]-4-carboxylic acid

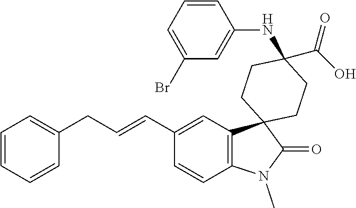 [SMILES: CN1c2ccc(/C=C/Cc3ccccc3)cc2[C@]2(CC[C@](Nc3cccc(Br)c3)(C(=O)O)CC2)C1=O]

To a solution of Example 19A (51 mg, 0.09 mmol, 1 eq) in MeOH (2 mL) was added 2 M aq. NaOH solution (0.5 mL) and then heated at 40° C. for 3 h. The reaction was partially concentrated in vacuo and then diluted with water and acidified with 2 M aq. HCl solution. The precipitate was collected by filtration, dissolved in DCM, dried (MgSO₄) and concentrated in vacuo. Purification by flash chromatography (5 g silica cartridge) eluting with a stepped gradient of 0-5% MeOH in DCM followed by preparative HPLC at pH 9 afforded Example 19 as a white powder (2.7 mg, 4.95 μmol, 5%). LRMS calculated for C₃₀H₂₉N₂O₃Br: 544; found: 545 (M+H). ¹H NMR (400 MHz, DMSO-d₆) δ ppm: 7.36-7.29 (m, 4H), 7.29-7.19 (m, 3H), 6.99 (t, J=8.0 Hz, 1H), 6.94 (d, J=8.0 Hz, 1H), 6.87 (t, 1H), 6.68-6.63 (m, 2H), 6.50 (d, J=15.8 Hz, 1H), 6.31 (dt, J=15.8, 6.9 Hz, 1H), 3.52 (d, J=6.9 Hz, 2H), 3.10 (s, 3H), 2.62-2.54 (m, 2H), 2.01-1.86 (m, 4H), 1.64-1.53 (m, 2H).

### Example 20

### Example 20A 5-bromo-1,7-dimethyl-3H-indol-2-one

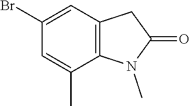

To a partial suspension of 5-bromo-7-methyl-2,3-dihydro-1H-indol-2-one (250 mg, 1.11 mmol, 1 eq) in THE (4 mL) was added MeOH (67 μL, 1.66 mmol, 1.5 eq) and PPh₃ (435 mg, 1.66 mmol, 1.5 eq) and then the mixture cooled in an ice bath. DIAD (327 μL, 1.66 mmol, 1.5 eq) was added dropwise and the reaction slowly allowed to warm to rt and stirred for 18 h. The mixture was partitioned between EtOAc and water and the organic phase dried (MgSO₄) and concentrated in vacuo. Purification by flash chromatography (10 g silica cartridge) eluting with a stepped gradient of 0-10% EtOAc in heptane, followed by flash chromatography (10 g silica cartridge) in DCM afforded Example 20A as a peach powder (49.5 mg, 0.21 mmol, 19%). ¹H NMR (400 MHz, DMSO-d₆) δ ppm: 7.28-7.24 (m, 2H), 3.55 (s, 2H), 3.38 (s, 3H), 2.53 (s, 3H).

### Example 20B 5″-bromo-1″,7″-dimethyldispiro[1,3-dioxolane-2,1′-cyclohexane-4′,3″-indole]-2″-one

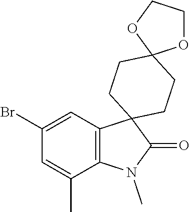 [SMILES: Cc1cc(Br)cc2c1N(C)C(=O)C21CCC2(CC1)OCCO2]

Example 20A (49.5 mg, 0.21 mmol, 1 eq) and Preparation 1b (59 mg, 0.21 mmol, 1 eq) in DMF (2 mL) was treated as described in General procedure 8a and stirred at rt for 18 h. The reaction was quenched with sat. aq. NH₄Cl solution, diluted with sat. aq. NaHCO₃ solution and extracted with EtOAc. The organic phase was washed with brine, dried (MgSO₄) and concentrated in vacuo. Purification by flash chromatography (5 g silica cartridge) eluting with a stepped gradient of 0-20% EtOAc in heptane afforded Example 20B as a brown solid (20 mg, 0.05 mmol, 26%) that was used directly in the subsequent step without further purification. LRMS calculated for C₁₇H₂₀NO₃Br: 365; found: 366 (M+H).

### Example 20C 5′-bromo-1′,7′-dimethylspiro[cyclohexane-1,3′-indole]-2′,4-dione

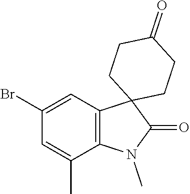 [SMILES: Cc1cc(Br)cc2c1N(C)C(=O)C21CCC(=O)CC1]

A solution of Example 20B (20 mg, 0.05 mmol, 1 eq) in acetone (2 mL) was treated as described in General procedure 9 and stirred at 45° C. for 2 h. The reaction was allowed to cool to rt and partitioned between DCM and water. The organic phase was washed with brine, dried (MgSO₄) and concentrated in vacuo to afford Example 20C as a brown gum (20 mg, 0.06 mmol, quant.) that was used directly in the subsequent step without further purification. LRMS calculated for C₁₅H₁₆NO₂Br: 321; found: 322 (M+H).

### Example 20 (1r,4r)-5′-bromo-4-(3-bromoanilino)-1′,7′-dimethyl-2′-oxo-1′,2′-dihydrospiro[cyclohexane-1,3′-indole]-4-carboxylic acid

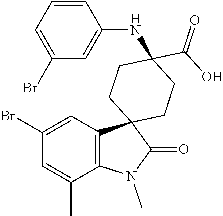 [SMILES: Cc1cc(Br)cc2c1N(C)C(=O)[C@]21CC[C@@](Nc2cccc(Br)c2)(C(=O)O)CC1]

A solution of Example 20C (20 mg, 0.06 mmol, 1 eq) in THE (3 mL) was treated as described in General procedure 10 using 3-bromoaniline (7 μL, 0.06 mmol, 1 eq) and then stirred at rt for 48 h. The reaction was diluted with water, acidified with 2 M aq. HCl solution and extracted with DCM. The organic phase was loaded onto a DCM-wet PE-AX cartridge (5 g), washed successively with DCM, MeOH and eluted with 5% HCOOH in DCM, and concentrated in vacuo. Purification by preparative HPLC at pH 4 afforded a single diastereoisomer, Example 20 as a white powder (2.9 mg, 0.01 mmol, 9%). LRMS calculated for C₂₂H₂₂N₂O₃Br₂: 520; found: 521 (M+H). ¹H NMR (400 MHz, DMSO-d₆) δ ppm: 12.72 (br s, 1H), 7.25 (s, 2H), 7.03 (t, J=8.1 Hz, 1H), 6.85 (t, J=2.1 Hz, 1H), 6.70 (d, J=7.8 Hz, 1H), 6.64 (dd, 1H), 6.19 (br s, 1H), 3.37 (s, 3H), 2.61-2.54 (m, 2H), 2.53 (s, 3H), 2.04-1.91 (m, 4H), 1.56-1.47 (m, 2H).

### Example 22

### Example 22A 1″H-dispiro[1,3-dioxolane-2,1′-cyclohexane-4′,3″-indole]-2″-one

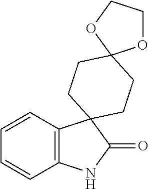 [SMILES: O=C1Nc2ccccc2C12CCC1(CC2)OCCO1]

To a suspension of 1″,2″-dihydrospiro[cyclohexane-1,3″-indole]-2″,4-dione (1 g, 4.65 mmol, 1 eq) in toluene (10 mL) was added PTSA (44 mg, 0.23 mmol, 0.05 eq) and ethylene glycol (0.39 mL, 6.97 mmol, 1.5 eq) and the reaction stirred at 90° C. for 3 h. The reaction was cooled to rt, diluted with EtOAc and washed with sat. aq. NaHCO₃ solution followed by brine. The organic phase was dried (MgSO₄) and concentrated in vacuo and then triturated with Et₂O. The precipitate was collected by filtration to afford Example 22A as a cream powder (904 mg, 3.49 mmol, 75%). LRMS calculated for C₁₅H₁₇NO₃: 259; found: 260 (M+H). ¹H NMR (400 MHz, DMSO-d₆) δ ppm: 10.36 (s, 1H), 7.31 (d, J=7.4 Hz, 1H), 7.18 (td, J=7.7, 1.2 Hz, 1H), 6.96 (td, J=7.5, 1.1 Hz, 1H), 6.85 (d, J=7.7 Hz, 1H), 3.94 (d, J=1.7 Hz, 4H), 2.14-2.04 (m, 2H), 1.88-1.67 (m, 6H).

### Example 22B tert-butyl 2″-oxodispiro[1,3-dioxolane-2,1′-cyclohexane-4′,3″-indole]-1″-carboxylate

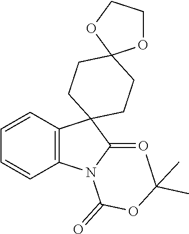 [SMILES: CC(C)(C)OC(=O)N1C(=O)C2(CCC3(CC2)OCCO3)c2ccccc21]

To a solution of Example 22A (904 mg, 3.49 mmol, 1 eq) in DCM (20 mL) was added TEA (0.97 mL, 6.97 mmol, 2 eq) and DMAP (21 mg, 0.17 mmol, 0.05 eq) followed by Boc₂O, (0.91 g, 4.18 mmol, 1.2 eq) and the reaction stirred at rt for 18 h. The reaction was diluted with water, extracted with DCM and the organic phase washed with sat. aq. NaHCO₃ solution, dried (MgSO₄) and concentrated in vacuo. Purification by flash chromatography (10 g silica cartridge) eluting with a stepped gradient of 0-20% EtOAc in heptane afforded Example 22B as a cream solid (1.15 g, 3.21 mmol, 92%). LRMS calculated for C₂₀H₂₅NO₅: 359; found 260 (M+H-Boc). ¹H NMR (400 MHz, DMSO-d₆) δ ppm: 7.75 (dd, J=8.3, 1.0 Hz, 1H), 7.39 (dd, J=7.4, 1.2 Hz, 1H), 7.36-7.31 (m, 1H), 7.19 (td, J=7.5, 1.1 Hz, 1H), 3.98-3.90 (m, 4H), 2.11 (dt, J=13.8, 7.2 Hz, 2H), 1.87 (dd, J=8.0, 4.5 Hz, 4H), 1.73 (dt, J=13.3, 5.0 Hz, 2H), 1.58 (s, 9H).

### Example 22C tert-butyl 2″-hydroxy-2″-methyldispiro[1,3-dioxolane-2,1′-cyclohexane-4′,3″-indole]-1″-carboxylate

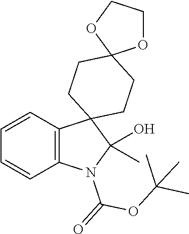 [SMILES: CC(C)(C)OC(=O)N1c2ccccc2C2(CCC3(CC2)OCCO3)C1(C)O]

A solution of Example 22B (500 mg, 1.39 mmol, 1 eq) in THE (10 mL) cooled to −78° C., was treated with bromo(methyl)magnesium (3 M, 0.7 mL, 2.09 mmol, 1.5 eq) and then slowly allowed to warm to rt and stirred for 1 h. The reaction was quenched with sat. aq. NH₄Cl solution and partitioned between EtOAc and water. The organic phase was dried (MgSO₄) and concentrated in vacuo. Purification by flash chromatography (10 g silica cartridge) eluting with a stepped gradient of 0-20% EtOAc in heptane afforded a racemic mixture, Example 22C as a white solid (320 mg, 0.85 mmol, 61%). ¹H NMR (400 MHz, DMSO-d₆) δ ppm: 7.54 (d, J=8.1 Hz, 1H), 7.30 (dd, J=7.6, 1.3 Hz, 1H), 7.15 (td, 1H), 6.95 (td, J=7.5, 1.1 Hz, 1H), 5.99 (s, 1H), 3.90 (dd, J=4.2, 2.1 Hz, 4H), 2.05-1.93 (m, 2H), 1.89-1.79 (m, 1H), 1.78-1.64 (m, 5H), 1.55 (s, 9H), 1.52 (s, 3H).

### Example 22D 2″-methyldispiro[1,3-dioxolane-2,1′-cyclohexane-4′,3″-indole]

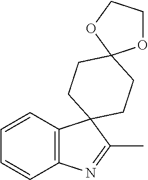 [SMILES: CC1=Nc2ccccc2C12CCC1(CC2)OCCO1]

A solution of Example 22C (320 mg, 0.85 mmol, 1 eq) in DCM (5 mL) was treated with TFA (0.5 mL) and then stirred at rt for 30 min. The reaction was diluted with water, basified with 2 M aq. NaOH solution and extracted with DCM. The organic extracts were dried (MgSO₄) and concentrated in vacuo to afford Example 22D as a colourless oil (233 mg, 0.91 mmol, quant.). LRMS calculated for C₁₆H₁₉NO₂: 257; found: 258 (M+H). ¹H NMR (400 MHz, DMSO-d₆) δ ppm: 7.66 (dt, J=7.4, 0.9 Hz, 1H), 7.48 (dd, J=7.7, 1.1 Hz, 1H), 7.34 (td, J=7.6, 1.1 Hz, 1H), 7.18 (td, J=7.4, 1.2 Hz, 1H), 3.98 (d, J=1.3 Hz, 4H), 2.22 (s, 3H), 2.08 (ddd, J=9.7, 5.4, 2.3 Hz, 4H), 1.83 (dd, J=10.2, 2.9 Hz, 2H), 1.18 (dd, J=9.2, 2.5 Hz, 2H).

### Example 22E 2′-methylspiro[cyclohexane-1,3′-indole]-4-one

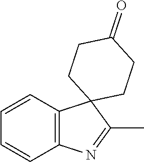 [SMILES: CC1=Nc2ccccc2C12CCC(=O)CC2]

A solution of Example 22D (127 mg, 0.49 mmol, 1 eq) in acetone (5 mL) was treated as described in General procedure 9 and stirred at rt for 18 h and then at 45° C. for a further 4 h. The reaction was allowed to cool to rt and partitioned between DCM and water. The organic phase was washed with brine, dried (MgSO₄) and concentrated in vacuo to afford Example 22E as a yellow oil (59 mg, 0.28 mmol, 56%) that was used directly in the subsequent step without further purification. LRMS calculated for C₁₄H₁₅NO: 213; found 214 (M+H). ¹H NMR (400 MHz, DMSO-d₆) δ ppm: 7.93 (d, 1H), 7.52 (d, J=7.4 Hz, 1H), 7.38 (td, J=7.6, 1.2 Hz, 1H), 7.21 (td, J=7.5, 1.3 Hz, 1H), 2.97-2.86 (m, 2H), 2.46-2.30 (m, 4H), 2.26 (s, 3H), 1.52-1.44 (m, 2H).

### Example 22 (1r,4r)-4-(3-bromoanilino)-2′-methylspiro[cyclohexane-1,3′-indole]-4-carboxylic acid

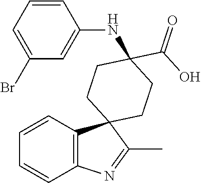 [SMILES: CC1=Nc2ccccc2[C@]12CC[C@](Nc1cccc(Br)c1)(C(=O)O)CC2]

A solution of Example 22E (59 mg, 0.28 mmol, 1 eq) in THE (3 mL) was treated as described in General procedure 10 using 3-bromoaniline (30 μL, 0.28 mmol, 1 eq) and then stirred at rt for 48 h. The reaction was diluted with water, acidified with 2 M aq. HCl solution and extracted with DCM. The organic phase was loaded onto a DCM-wet PE-AX cartridge (5 g), washed successively with DCM and MeOH and eluted with 5% HCOOH in DCM and concentrated in vacuo. Trituration with DCM afforded a single diastereoisomer, Example 22 as a cream powder (5.1 mg, 0.01 mmol, 4%). Purification of the filtrate by preparative HPLC at pH 9 afforded additional Example 22 as a cream powder (6 mg, 0.01 mmol, 5%). LRMS calculated for C₂₁H₂₁N₂O₂Br: 412; found: 413 (M+H). ¹H NMR (400 MHz, DMSO-d₆) δ ppm: 7.70 (d, J=7.3 Hz, 1H), 7.47 (d, J=7.2 Hz, 1H), 7.34 (td, J=7.5, 1.1 Hz, 1H), 7.20 (td, 1H), 7.04 (t, J=8.1 Hz, 1H), 6.87 (t, J=2.1 Hz, 1H), 6.73 (d, J=8.0 Hz, 1H), 6.67 (dd, J=8.2, 2.2 Hz, 1H), 2.22 (s, 3H), 2.10-1.96 (m, 4H), 1.35-1.26 (m, 2H).

### Example 23 and Example 24

### Example 23A 2″-methyl-1″,2″-dihydrodispiro[1,3-dioxolane-2,1′-cyclohexane-4′,3″-indole]

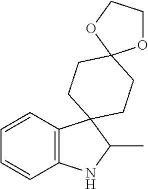 [SMILES: CC1Nc2ccccc2C12CCC1(CC2)OCCO1]

NaBH₄ (137 mg, 3.62 mmol, 4 eq) was added to a solution of Example 22D (233 mg, 0.91 mmol, 1 eq) in MeOH (5 mL) and then stirred at rt for 30 min. The reaction was concentrated in vacuo and the residue partitioned between DCM and water. The organic phase was washed with brine, dried (MgSO₄) and concentrated in vacuo to afford a racemic mixture, Example 23A as a colourless oil which solidified on standing (210 mg, 0.81 mmol, 89%). LRMS calculated for C₁₆H₂₁NO₂: 259; found: 260 (M+H). ¹H NMR (400 MHz, DMSO-d₆) δ ppm: 7.01 (dd, J=7.3, 1.2 Hz, 1H), 6.90 (td, J=7.6, 1.3 Hz, 1H), 6.53 (td, J=7.4, 1.1 Hz, 1H), 6.47 (dd, J=7.7, 0.9 Hz, 1H), 5.39 (s, 1H), 3.93-3.85 (m, 4H), 3.60 (q, J=6.3 Hz, 1H), 3.33 (s, 3H), 1.87-1.64 (m, 6H), 1.61-1.36 (m, 2H), 1.00 (d, J=6.3 Hz, 3H).

### Example 23B 1″,2″-dimethyl-2″H-dispiro[1,3-dioxolane-2,1′-cyclohexane-4′,3″-indole]

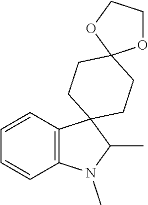 [SMILES: CC1N(C)c2ccccc2C12CCC1(CC2)OCCO1]

To a solution of the racemic mixture Example 23A (210 mg, 0.81 mmol, 1 eq) in DMF (3 mL), cooled in an ice bath under a N₂ atmosphere, was added NaH (60% dispersion; 49 mg, 1.21 mmol, 1.5 eq) and the mixture stirred for 5 min. MeI (101 μL, 1.62 mmol, 2 eq) was added and the reaction slowly allowed to warm to rt and stirred for 1.5 h. Further MeI (50 μL, 0.81 mmol, 1 eq) was added and stirring continued for 1 h. The reaction was quenched with water and extracted with EtOAc. The organic extracts were washed with brine, dried (MgSO₄) and concentrated in vacuo. Purification by flash chromatography (5 g silica cartridge) eluting with a stepped gradient of 0-10% EtOAc in heptane afforded a racemic mixture, Example 23B as a colourless oil (113 mg, 0.41 mmol, 51%). LRMS calculated for C₁₇H23NO₂: 273; found: 274 (M+H). ¹H NMR (400 MHz, DMSO-d₆) δ ppm: 7.11 (dd, J=7.3, 1.2 Hz, 1H), 7.02 (td, J=7.6, 1.3 Hz, 1H), 6.60 (td, J=7.4, 1.0 Hz, 1H), 6.44 (dd, J=7.8, 0.9 Hz, 1H), 3.93-3.86 (m, 4H), 3.24 (q, J=6.5 Hz, 1H), 2.66 (s, 3H), 1.90-1.81 (m, 1H), 1.80-1.61 (m, 5H), 1.60-1.50 (m, 2H), 1.02 (d, J=6.5 Hz, 3H).

### Example 23C 1′,2′-dimethyl-2′H-spiro[cyclohexane-1,3′-indole]-4-one

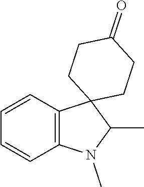 [SMILES: CC1N(C)c2ccccc2C12CCC(=O)CC2]

A solution of Example 23B (113 mg, 0.41 mmol, 1 eq) in acetone (3 mL) was treated as described in General procedure 9 and then stirred at 50° C. for 1 h. The reaction was cooled to rt, diluted with water and extracted with DCM. The organic phase was dried (MgSO₄) and concentrated in vacuo to afford a racemic mixture, Example 23C as a colourless oil (96.4 mg, 0.42 mmol, quant.) that was used directly in the subsequent step without further purification. LRMS calculated for C₁₅H₁₉NO: 229; found 230 (M+H). ¹H NMR (400 MHz, DMSO-d₆) δ ppm: 7.27 (dd, J=7.3, 1.2 Hz, 1H), 7.06 (td, J=7.6, 1.2 Hz, 1H), 6.63 (td, J=7.4, 1.0 Hz, 1H), 6.51 (dd, J=7.8, 0.9 Hz, 1H), 3.32 (q, J=6.4 Hz, 1H), 2.69 (s, 3H), 2.64-2.47 (m, 2H), 2.45-2.36 (m, 1H), 2.35-2.25 (m, 1H), 2.05-1.90 (m, 2H), 1.89-1.79 (m, 2H), 1.12 (d, J=6.4 Hz, 3H).

### Example 23 (1r,4r)-4-(3-bromoanilino)-1′,2′-dimethyl-1′,2′-dihydrospiro[cyclohexane-1,3′-indole]-4-carboxylic acid

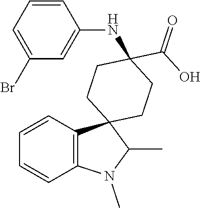

### Example 24 (1s,4s)-4-(3-bromoanilino)-1′,2′-dimethyl-1′,2′-dihydrospiro[cyclohexane-1,3′-indole]-4-carboxylic acid

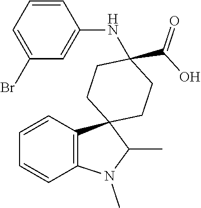

A solution of Example 23C (14 mg, 0.06 mmol, 1 eq) in THE (3 mL) was treated as described in General procedure 10 using 3-bromoaniline (7 μL, 0.06 mmol, 1 eq) and the mixture was stirred at rt for 18 h, to afford a 1:1 mixture of racemic diastereoisomers. The mixture was diluted with water and acidified with 2 M aq. HCl solution and extracted with DCM. The organic extract was loaded onto a DCM-wet PE-AX cartridge (5 g) and washed successively with DCM, MeOH and eluted with 10% HCOOH in DCM and then the diastereoisomers were separated by preparative HPLC at pH 4. The racemic diastereoisomer eluting earlier was collected as Example 23 as a cream powder (3.3 mg, 0.01 mmol, 13%). LRMS calculated for C₂₂H₂₅N₂O₂Br: 428; found: 429 (M+H). ¹H NMR (400 MHz, DMSO-d₆) δ ppm: 12.71 (br s, 1H), 7.25 (d, J=7.2 Hz, 1H), 7.05 (td, J=7.7, 1.2 Hz, 1H), 7.01 (t, J=8.1 Hz, 1H), 6.78 (t, J=2.1 Hz, 1H), 6.70-6.62 (m, 2H), 6.58-6.54 (m, 1H), 6.51 (app d, J=7.9 Hz, 1H), 6.20 (br s, 1H), 2.92 (q, J=6.5 Hz, 1H), 2.63 (s, 3H), 2.42-2.32 (m, 1H), 2.15-2.05 (m, 1H), 2.03-1.95 (m, 1H), 1.97-1.70 (m, 3H), 1.56-1.47 (m, 1H), 1.32-1.23 (m, 1H), 1.10 (d, J=6.5 Hz, 3H).

The racemic diastereoisomer eluting later was collected as Example 24 as a white powder (3 mg, 0.01 mmol, 11%). LRMS calculated for C₂₂H₂₅N₂O₂Br: 428; found: 429 (M+H). ¹H NMR (400 MHz, DMSO-d₆) δ ppm: 7.06-6.95 (m, 3H), 6.81 (t, J=2.1 Hz, 1H), 6.66-6.55 (m, 3H), 6.39 (d, J=7.8 Hz, 1H), 6.14 (br s, 1H), 2.68 (s, 3H), 2.19-2.09 (m, 1H), 2.04-1.92 (m, 2H), 1.91-1.78 (m, 2H), 1.71-1.50 (m, 3H), 1.00 (d, J=6.4 Hz, 3H).

### Example 25

### Example 25A tert-butyl 2″-hydroxy-2″-(2-phenylethyl)dispiro[1,3-dioxolane-2,1′-cyclohexane-4′,3″-indole]-1″-carboxylate

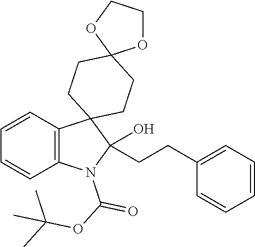 [SMILES: CC(C)(C)OC(=O)N1c2ccccc2C2(CCC3(CC2)OCCO3)C1(O)CCc1ccccc1]

To a solution of Example 22B (484 mg, 1.35 mmol, 1 eq) in THE (10 mL) cooled to −78° C. under a N₂ atmosphere, was added phenethylmagnesium bromide (0.5 M, 4.04 mL, 2.02 mmol, 1.5 eq) dropwise. The mixture was slowly allowed to warm to rt and stirred for 2 h. The reaction was quenched with sat. aq. NH₄Cl solution, diluted with water and extracted with EtOAc. The organic phase was dried (MgSO₄) and concentrated in vacuo. Purification by flash chromatography (20 g silica cartridge) eluting with a gradient of 0-10% EtOAc in heptane afforded a racemic mixture, Example 25A as a white solid (189 mg, 0.41 mmol, 30%) that was used directly in the subsequent step without further purification.

### Example 25B 2″-(2-phenylethyl)dispiro[1,3-dioxolane-2,1′-cyclohexane-4′,3″-indole]

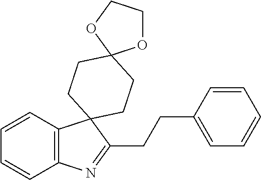 [SMILES: c1ccc(CCC2=Nc3ccccc3C23CCC2(CC3)OCCO2)cc1]

To a solution of Example 25A (241 mg, 0.52 mmol, 1 eq) in DCM (5 mL) was added TFA (0.5 mL) and the mixture stirred at rt for 1 h. The reaction was diluted with water, basified with 2 M aq. NaOH solution and extracted with DCM. The organic phase was dried (MgSO₄) and concentrated in vacuo. Purification by flash chromatography (5 g silica cartridge) eluting with a stepped gradient of 0-5% EtOAc in heptane afforded Example 25B as a yellow oil (126 mg, 0.36 mmol, 70%). LRMS calculated for C₂₃H₂₅NO₂: 347; found: 348 (M+H). ¹H NMR (400 MHz, DMSO-d₆) δ ppm: 7.65 (dd, J=7.4, 1.0 Hz, 1H), 7.56 (dd, J=7.7, 1.0 Hz, 1H), 7.36 (td, J=7.6, 1.1 Hz, 1H), 7.33-7.26 (m, 4H), 7.22-7.17 (m, 2H), 3.95 (s, 4H), 3.09 (dd, J=8.5, 7.0 Hz, 2H), 2.80 (dd, J=8.6, 7.0 Hz, 2H), 2.09-2.02 (m, 4H), 1.83-1.77 (m, 2H), 1.13-1.08 (m, 2H).

### Example 25C 2″-(2-phenylethyl)-1″,2″-dihydrodispiro[1,3-dioxolane-2,1′-cyclohexane-4′,3″-indole]

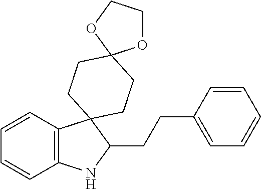 [SMILES: c1ccc(CCC2Nc3ccccc3C23CCC2(CC3)OCCO2)cc1]

NaBH₄ (93 mg, 2.44 mmol, 5 eq) was added to a solution of Example 25B (170 mg, 0.49 mmol, 1 eq) in MeOH (5 mL) and then stirred at rt for 5 h. The reaction was concentrated in vacuo and the residue dissolved in DCM and washed with water. The organic phase was dried (MgSO₄) and concentrated in vacuo. Purification by flash chromatography (5 g silica cartridge) eluting with a stepped gradient of 0-5% EtOAc in heptane afforded a racemic mixture, Example 25C as a colourless oil (115 mg, 0.33 mmol, 67%). LRMS calculated for C₂₃H27NO₂: 349; found: 350 (M+H). ¹H NMR (400 MHz, DMSO-d₆) δ ppm: 7.32-7.22 (m, 4H), 7.21-7.15 (m, 1H), 7.04 (d, J=7.6 Hz, 1H), 6.93 (td, J=7.6, 1.2 Hz, 1H), 6.57-6.52 (m, 2H), 5.87 (s, 1H), 3.91-3.83 (m, 4H), 2.89-2.79 (m, 1H), 2.65-2.55 (m, 1H), 1.81-1.42 (m, 10H).

### Example 25D 1″-methyl-2″-(2-phenylethyl)-2″H-dispiro[1,3-dioxolane-2,1′-cyclohexane-4′,3″-indole]

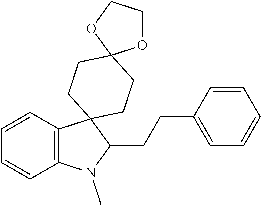 [SMILES: CN1c2ccccc2C2(CCC3(CC2)OCCO3)C1CCc1ccccc1]

To a solution of Example 25C (115 mg, 0.33 mmol, 1 eq) in DMF (2 mL) cooled in an ice bath under a N₂ atmosphere, was added NaH (60% dispersion; 12 mg, 0.49 mmol, 1.5 eq) and the mixture stirred for 30 min. MeI (23 μL, 0.36 mmol, 1.1 eq) was added and the reaction stirred at rt for 3 h. Additional NaH (12 mg, 0.49 mmol, 1.5 eq) followed by MeI (23 μL, 0.36 mmol, 1.1 eq) was added and stirring continued for 18 h. The reaction was diluted with water and extracted with DCM. The organic phase was dried (MgSO₄) and concentrated in vacuo. Purification by flash chromatography (5 g silica cartridge) eluting with a stepped gradient of 0-10% EtOAc in heptane afforded a racemic mixture, Example 25D as a colourless oil (54 mg, 0.15 mmol, 45%). LRMS calculated for C₂₄H₂₉NO₂: 363; found: 364 (M+H). ¹H NMR (400 MHz, DMSO-d₆) δ ppm: 7.30-7.25 (m, 2H), 7.21-7.16 (m, 3H), 7.11 (dd, J=7.3, 1.2 Hz, 1H), 7.02 (td, J=7.6, 1.2 Hz, 1H), 6.58 (td, J=7.5, 1.1 Hz, 1H), 6.46 (dd, J=7.8, 1.1 Hz, 1H), 3.92-3.88 (m, 4H), 3.24 (t, J=4.9 Hz, 1H), 2.83 (s, 3H), 2.60-2.46 (m, 2H), 1.97-1.47 (m, 10H).

### Example 25E 1′-methyl-2′-(2-phenylethyl)-2′H-spiro[cyclohexane-1,3′-indole]-4-one

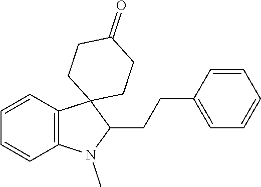 [SMILES: CN1c2ccccc2C2(CCC(=O)CC2)C1CCc1ccccc1]

A solution of Example 25D (54 mg, 0.15 mmol, 1 eq) in acetone (3 mL) was treated as described in General procedure 9 and then stirred at 45° C. for 4 h. The reaction was cooled to rt, diluted with water and extracted with DCM. The organic phase was dried (MgSO₄) and concentrated in vacuo to afford a racemic mixture, Example 25E as a colourless oil (47 mg, 0.15 mmol, 99%) that was used directly in the subsequent step without further purification. LRMS calculated for C₂₂H₂₅NO: 319; found: 320 (M+H).

### Example 25 4-(3-bromoanilino)-1′-methyl-2′-(2-phenylethyl)-1′,2′-dihydrospiro[cyclohexane-1,3′-indole]-4-carboxylic acid, diastereoisomer 1

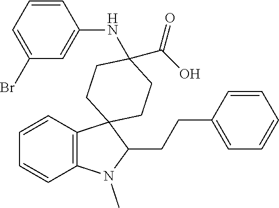 [SMILES: CN1c2ccccc2C2(CCC(Nc3cccc(Br)c3)(C(=O)O)CC2)C1CCc1ccccc1]

A solution of Example 25E (47 mg, 0.15 mmol, 1 eq) in THE (3 mL) was treated as described in General procedure 10 using 3-bromoaniline (16 μL, 0.15 mmol, 1 eq) and the mixture allowed to warm to rt and stirred for 18 h. A mixture of diastereoisomers was obtained. The mixture was diluted with water, acidified with 2 M aq. HCl solution and extracted with DCM. The organic phase was loaded onto a DCM-wet PE-AX cartridge (5 g) and washed successively with DCM and MeOH and eluted with 5% HCOOH in DCM. Purification by flash chromatography (5 g silica cartridge) eluting with a stepped gradient of 0-20% EtOAc in heptane was followed by preparative HPLC at pH 9 and subsequently pH 4 to separate the diastereoisomers. The diastereoisomer eluting earlier was dissolved in DCM and washed with dilute aq. HCl solution. The organic phase was dried (MgSO₄), concentrated and dried in vacuo to afford a single pair of enantiomers, Example 25 as a cream powder (4.9 mg, 0.01 mmol, 6%). LRMS calculated for C₂₉H₃₁N₂O₂Br: 518; found: 519 (M+H). ¹H NMR (400 MHz, DMSO-d₆) δ ppm: 12.71 (br s, 1H), 7.31-7.21 (m, 5H), 7.21-7.14 (m, 1H), 7.08-6.98 (m, 2H), 6.81 (t, J=2.0 Hz, 1H), 6.69 (d, J=8.1 Hz, 1H), 6.64 (td, J=7.4, 1.0 Hz, 1H), 6.57-6.50 (m, 2H), 6.33 (br s, 1H), 2.94-2.89 (m, 1H), 2.80-2.71 (m, 4H), 2.70-2.60 (m, 1H), 2.45-2.34 (m, 1H), 2.16-1.76 (m, 7H), 1.58-1.51 (m, 1H), 1.37-1.30 (m, 1H).

### Example 26 (1r,4r)-4-(3-bromoanilino)-1′-formyl-2′-methyl-1′,2′-dihydrospiro[cyclohexane-1,3′-indole]-4-carboxylic acid

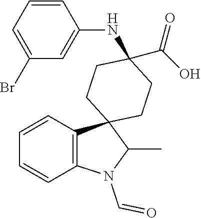

A solution of Example 22 (23 mg, 0.06 mmol, 1 eq) in MeOH (2 mL) was treated with NaBH₄ (11 mg, 0.28 mmol, 5 eq) and then stirred at rt for 1 h. The reaction was quenched with water and then washed with DCM. The aq. phase was extracted with EtOAc and the organic extracts dried (MgSO₄) and concentrated in vacuo. The residue was dissolved in DCM and loaded onto a DCM-wet PE-AX cartridge (5 g), washed successively with DCM, MeOH and eluted with 10% HCOOH in DCM, and concentrated in vacuo. Further purification by preparative HPLC at pH 4 afforded an enantiomer pair, Example 26 as a white powder (3.7 mg, 0.01 mmol, 15%). LRMS calculated for C₂₂H₂₃N₂O₃Br: 442; found: 443 (M+H). ¹H NMR (400 MHz, DMSO-d₆) δ ppm (4:6 mixture of amide rotamers): 12.75 (br s, 1H), 8.98 (s, 0.6H), 8.57 (s, 0.4H), 7.84 (d, J=7.7 Hz, 0.4H), 7.41 (d, J=7.8 Hz, 0.6H), 7.26-7.17 (m, 2H), 7.13-7.07 (m, 1H), 7.05-6.99 (m, 1H), 6.80 (t, J=2.1 Hz, 1H), 6.72-6.66 (m, 1H), 6.60-6.53 (m, 1H), 6.26 (br s, 1H), 4.61-4.49 (m, 1H), 2.30-2.13 (m, 2H), 2.07-1.87 (m, 2H), 1.83-1.72 (m, 1H), 1.71-1.62 (m, 1H), 1.58-1.47 (m, 1H), 1.45-1.30 (m, 1H), 1.19 (d, 6.6 Hz, 1.2H), 1.11 (d, J=6.6 Hz, 1.8H).

### Example 27

### Example 27A tert-butyl 5″-bromo-2″-oxodispiro[1,3-dioxolane-2,1′-cyclohexane-4′,3″-indole]-1″-carboxylate

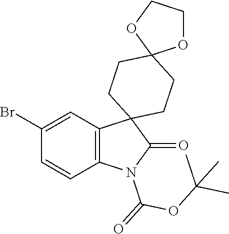 [SMILES: CC(C)(C)OC(=O)N1C(=O)C2(CCC3(CC2)OCCO3)c2cc(Br)ccc21]

To a solution of Example 6A (112 mg, 0.33 mmol, 1 eq), DMAP (2 mg, 0.02 mmol, 0.05 eq) and TEA (92 μL, 0.66 mmol, 2 eq) in DCM (3 mL) was added Boc₂O, (108 mg, 0.5 mmol, 1.5 eq) and the reaction stirred at rt for 6 h. The mixture was partitioned between DCM and water and the organic phase was washed with sat. aq. NaHCO₃ solution, brine, dried (MgSO₄) and concentrated in vacuo. Purification by flash chromatography (5 g silica cartridge) eluting with a stepped gradient of 0-1% MeOH in DCM followed by trituration in Et₂O afforded Example 27A as a cream powder (63 mg, 0.14 mmol, 43%). LRMS calculated for C₂₀H₂₄NO₅Br: 437; found: 338 (M+H-Boc). ¹H NMR (400 MHz, DMSO-d₆) δ ppm: 7.73-7.68 (m, 1H), 7.56-7.50 (m, 2H), 3.97-3.91 (m, 4H), 2.16-2.06 (m, 2H), 1.93-1.85 (m, 4H), 1.74-1.64 (m, 1H), 1.57 (s, 9H).

### Example 27B tert-butyl 5″-bromo-2″-hydroxy-2″-methyldispiro[1,3-dioxolane-2,1′-cyclohexane-4′,3″-indole]-1″-carboxylate

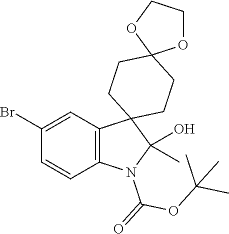 [SMILES: CC(C)(C)OC(=O)N1c2ccc(Br)cc2C2(CCC3(CC2)OCCO3)C1(C)O]

Example 27A (82 mg, 0.19 mmol, 1 eq) in THE (3 mL) was cooled to −78° C., treated with bromo(methyl)magnesium (3 M, 94 μL, 0.28 mmol, 1.5 eq) and then slowly allowed to warm to rt and stirred for 1 h. The reaction was quenched with sat. aq. NH₄Cl solution and partitioned between EtOAc and water. The organic phase was dried (MgSO₄) and concentrated in vacuo.

Purification by flash chromatography (5 g silica cartridge) eluting with a stepped gradient of 0-20% EtOAc in heptane afforded a racemic mixture, Example 27B as a colourless solid (63 mg, 0.14 mmol, 74%). ¹H NMR (400 MHz, DMSO-d₆) δ ppm: 7.51 (d, J=8.6 Hz, 1H), 7.38-7.31 (m, 2H), 6.12 (s, 1H), 3.93-3.86 (m, 4H), 2.06-1.92 (m, 2H), 1.90-1.79 (m, 1H), 1.74-1.62 (m, 4H), 1.58-1.48 (m, 13H).

### Example 27C 5″-bromo-2″-methyldispiro[1,3-dioxolane-2,1′-cyclohexane-4′,3″-indole]

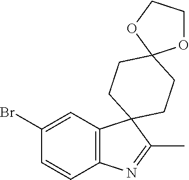 [SMILES: CC1=Nc2ccc(Br)cc2C12CCC1(CC2)OCCO1]

A solution of Example 27B (63 mg, 0.14 mmol, 1 eq) in DCM (3 mL) was treated with TFA (0.3 mL) and then stirred at rt for 2 h. The reaction was diluted with water, basified with 2 M aq. NaOH solution and extracted with DCM. The combined organic extracts were dried (MgSO₄) and concentrated in vacuo to afford Example 27C as a colourless gum (45 mg, 0.13 mmol, 97%) that was used directly in the subsequent step without further purification. LRMS calculated for C₁₆H₁₈NO₂Br: 335; found: 336 (M+H).

### Example 27D 5′-bromo-2′-methylspiro[cyclohexane-1,3′-indole]-4-one

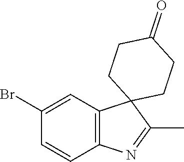 [SMILES: CC1=Nc2ccc(Br)cc2C12CCC(=O)CC2]

A solution of Example 27C (45 mg, 0.13 mmol, 1 eq) in acetone (3 mL) was treated as described in General procedure 9 and stirred at 50° C. for 3 h. The reaction was allowed to cool to rt, diluted with water and extracted with DCM. The organic phase was dried (MgSO₄) and concentrated in vacuo to afford Example 27D as a cream solid (29.4 mg, 0.1 mmol, 75%) that was used directly in the subsequent step without further purification. LRMS calculated for C₁₄H₁₄NOBr: 291; found: 292 (M+H).

### Example 27 (1r,4r)-5′-bromo-4-(3-bromoanilino)-2′-methylspiro[cyclohexane-1,3′-indole]-4-carboxylic acid

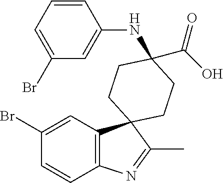 [SMILES: CC1=Nc2ccc(Br)cc2[C@]12CC[C@](Nc1cccc(Br)c1)(C(=O)O)CC2]

A solution of Example 27D (29.4 mg, 0.1 mmol, 1 eq) in THE (3 mL) was treated as described in General procedure 10 using 3-bromoaniline (11 μL, 0.1 mmol, 1 eq) and stirred at rt for 48 h. The reaction was diluted with water, acidified with 2 M aq. HCl solution and extracted with DCM. The organic phase was loaded onto a DCM-wet PE-AX cartridge (5 g), washed successively with DCM, MeOH and eluted with 5% HCOOH in DCM, and concentrated in vacuo. Purification by preparative HPLC at pH 4 afforded a single diastereoisomer, Example 27 as a white powder (2.8 mg, 0.01 mmol, 6%). LRMS calculated for C₂₁H₂₀N₂O₂Br₂: 490; found: 491 (M+H). ¹H NMR (400 MHz, DMSO-d₆) δ ppm: 7.80 (d, J=2.0 Hz, 1H), 7.54 (dd, J=8.2, 1.9 Hz, 1H), 7.43 (d, J=8.2 Hz, 1H), 7.03 (t, J=8.1 Hz, 1H), 6.87 (app s, 1H), 6.75-6.65 (m, 2H), 2.22 (s, 3H), 2.08-1.89 (m, 4H), 1.39-1.29 (m, 2H).

### Example 28 and Example 29

### Example 28A N-[(2-bromophenyl)methyl]-4-methyl-benzenesulfonamide

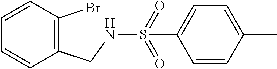 [SMILES: Cc1ccc(S(=O)(=O)NCc2ccccc2Br)cc1]

2-bromobenzylamine hydrochloride (4.33 g, 19.5 mmol) was dissolved in DCM (110 mL). TEA (6.0 mL, 42.8 mmol) and p-toluenesulfonyl chloride (4.1 g, 21.4 mmol) was added to the mixture and stirred at 0° C. for 30 min. The reaction mixture was washed with 1 M aq. HCl solution, 0.1 M aq. NaOH solution, and then with brine. The combined organic layers were dried over Na₂SO₄, filtered and the filtrate was concentrated under reduced pressure to give Example 28A. ¹H NMR (500 MHz, DMSO-d₆) δ ppm: 8.18 (t, 1H), 7.71 (m, 2H), 7.56 (dm, 1H), 7.43 (dm, 1H), 7.39 (m, 2H), 7.35 (m, 1H), 7.20 (m, 1H), 3.99 (d, 2H), 2.38 (s, 3H). HRMS calculated for C₁₄H₁₄BrNO₂S: 338.9929; found 361.9824 (M+Na).

### Example 28B 8-(3-chloroanilino)-1,4-dioxaspiro[4.5]decane-8-carboxylic acid

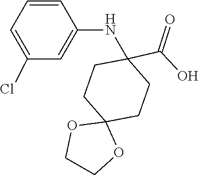 [SMILES: O=C(O)C1(Nc2cccc(Cl)c2)CCC2(CC1)OCCO2]

Using General procedure 10 and 1,4-dioxaspiro[4.5]decan-8-one as the appropriate ketone and 3-chloroaniline as the appropriate aniline Example 28B was obtained. ¹H NMR (500 MHz, DMSO-d₆) δ ppm: 12.66 (br s, 1H), 7.05 (t, 1H), 6.58-6.47 (m, 3H), 6.18 (br s, 1H), 3.88-3.82 (m, 4H), 2.03-1.95 (m, 4H), 1.71/1.55 (m+m, 4H). HRMS calculated for C₁₅H₁₈ClNO₄: 311.0924; found 312.1002 (M+H).

### Example 28C 1-(3-chloroanilino)-4-oxo-cyclohexanecarboxylic acid

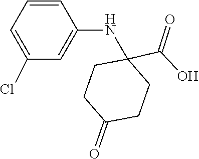 [SMILES: O=C1CCC(Nc2cccc(Cl)c2)(C(=O)O)CC1]

Using General procedure 9 and Example 28B as the appropriate ketone Example 28C was obtained. ¹H NMR (500 MHz, DMSO-d₆) δ ppm: 12.88 (br s, 1H), 7.09 (t, 1H), 6.65-6.51 (m, 3H), 6.45 (br s, 1H), 2.48/2.20 (m+m, 4H), 2.28/2.22 (m+m, 4H). HRMS calculated for C₁₃H₁₄ClNO₃: 267.0662; found 268.0731 (M+H).

### Example 28D ethyl 1-(3-chloroanilino)-4-oxo-cyclohexanecarboxylate

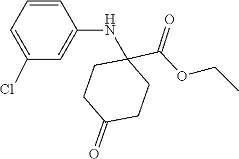 [SMILES: CCOC(=O)C1(Nc2cccc(Cl)c2)CCC(=O)CC1]

Example 28C (5.3 g, 20 mmol) was dissolved in DMF (100 mL). CsF (6.1 g, 40 mmol) and EtI (3.2 mL, 40 mmol) were added and the mixture was stirred at 35° C. overnight. The mixture was diluted with sat. aq. NaHCO₃ solution and extracted with EtOAc. The combined organic layers were dried over Na₂SO₄, filtered and the filtrate was concentrated under reduced pressure. The crude product was purified via flash chromatography using heptane and EtOAc as eluents to give Example 28D. ¹H NMR (500 MHz, DMSO-d₆) δ ppm: 7.10 (t, 1H), 6.64-6.46 (m, 3H), 6.51 (s, 1H), 4.12 (q, 2H), 2.49/2.21 (m+m, 4H), 2.30/2.23 (m+m, 4H), 1.09 (t, 3H). HRMS calculated for C₁₅H₁₈ClNO₃: 295.0975; found 296.1052 (M+H).

### Example 28E ethyl 1-(3-chloroanilino)-4-[(trifluoromethanesulfonyl)oxy]cyclohex-3-ene-1-carboxylate

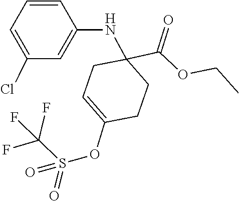 [SMILES: CCOC(=O)C1(Nc2cccc(Cl)c2)CC=C(OS(=O)(=O)C(F)(F)F)CC1]

Example 28D (825 mg, 2.8 mmol) was dissolved in dry THE (10 mL) and cooled to −78° C. under N₂. LiHMDS in THE (1 M, 8.3 mL, 8.3 mmol) was added to the mixture dropwise at −78° C. The mixture was stirred at −40° C. for 2 h. The mixture was cooled to −78° C. and a solution of 1,1,1-trifluoro-N-phenyl-N-(trifluoromethylsulfonyl)methanesulfonamide (1.99 g, 5.6 mmol) in dry THE (5 mL) was added dropwise. The resulting solution was allowed to warm to rt and stirred at rt overnight. It was quenched with sat. aq. NH₄Cl and extracted with EtOAc. The combined organic layers were washed with brine, dried over Na₂SO₄, filtered and the filtrate was concentrated under reduced pressure. The crude product was purified via flash chromatography using heptane and EtOAc as eluents to give Example 28E. ¹H NMR (500 MHz, DMSO-d₆) δ ppm: 7.08 (t, 1H), 6.60 (dm, 1H), 6.54 (dd, 1H), 6.46 (dm, 1H), 6.34 (br s, 1H), 5.85 (m, 1H), 4.11 (qm, 2H), 2.86/2.52 (br d+br d, 2H), 2.47/2.32 (m+m, 2H), 2.28/2.12 (m+m, 2H), 1.09 (t, 3H). HRMS calculated for C₁₆H₁₇ClF₃NO₅S: 427.0468; found 428.0543 (M+H).

### Example 28F ethyl 1-(3-chloroanilino)-4-(4,4,5,5-tetramethyl-1,3,2-dioxaborolan-2-yl)cyclohex-3-ene-1-carboxylate

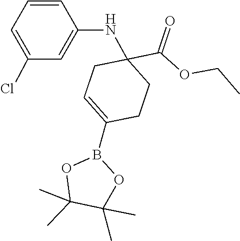 [SMILES: CCOC(=O)C1(Nc2cccc(Cl)c2)CC=C(B2OC(C)(C)C(C)(C)O2)CC1]

Example 28E (1.1 g. 2.5 mmol), 4,4,5,5-tetramethyl-2-(4,4,5,5-tetramethyl-1,3,2-dioxaborolan-2-yl)-1,3,2-dioxaborolane (635 mg, 2.5 mmol), KOAc (736 mg, 7.5 mmol) and Pd(dppf)Cl₂×DCM (408 mg, 0.5 mmol) were dissolved in 1,4-dioxane (10 mL) and stirred under N₂ at 40° C. for 2 h. The mixture was filtered through a pad of Celite and washed with EtOAc. The filtrate was washed with brine, dried over Na₂SO₄, filtered and the filtrate was concentrated under reduced pressure. The crude product was purified via flash chromatography using heptane and EtOAc as eluents to give Example 28F as a racemate. ¹H NMR (500 MHz, DMSO-d₆) δ ppm: 7.04 (t, 1H), 6.54 (dm, 1H), 6.51 (t, 1H), 6.42 (dm, 1H), 6.35 (m, 1H), 6.18 (s, 1H), 4.08 (q, 2H), 2.36/2.27 (m+m, 2H), 2.13-1.95 (m, 2H), 2.08/1.81 (m+m, 2H), 1.19 (s, 12H), 1.06 (t, 3H). HRMS calculated for C₂₁H₂₉BClNO₄: 405.1878; found 406.1956 (M+H).

### Example 28G ethyl 4-(3-chloroanilino)-2′-{[(4-methylbenzene-1-sulfonyl)amino]methyl}-2,3,4,5-tetrahydro[1,1′-biphenyl]-4-carboxylate

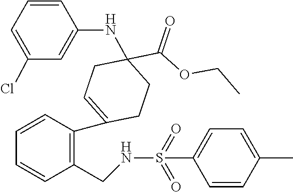 [SMILES: CCOC(=O)C1(Nc2cccc(Cl)c2)CC=C(c2ccccc2CNS(=O)(=O)c2ccc(C)cc2)CC1]

Example 28A (567 mg, 1.7 mmol), Example 28F (676 mg, 1.7 mmol), AtaPhos (118 mg, 0.17 mmol), and Cs₂CO₃ (1.36 g, 4.2 mmol) were dissolved in 1,4-dioxane (17 mL) and water (9 mL) and stirred under N₂ at 90° C. for 1 h. The mixture was diluted with water, brine and extracted with DCM. The combined organic layers were dried over Na₂SO₄, filtered and the filtrate was concentrated under reduced pressure. The crude product was purified via flash chromatography using heptane and EtOAc as eluents to give Example 28G. ¹H NMR (500 MHz, DMSO-d₆) δ ppm: 7.87 (br, 1H), 7.68 (dm, 2H), 7.37 (dm, 2H), 7.33 (m, 1H), 7.21 (m, 2H), 7.06 (t, 1H), 7.04 (m, 1H), 6.58 (dm, 1H), 6.56 (dd, 1H), 6.46 (dm, 1H), 6.21 (s, 1H), 5.37 (br, 1H), 4.09 (m, 2H), 3.89 (br, 2H), 2.70/2.32 (br d+br d, 2H), 2.38 (s, 3H), 2.22/2.05 (m+br d, 2H), 2.14/1.97 (m+m, 2H), 1.09 (t, 3H). HRMS calculated for C₂₉H₃₁ClN₂O₄S: 538.1693; found 539.177 (M+H).

### Example 28H ethyl 4-(3-chloroanilino)-2′-(4-methylbenzene-1-sulfonyl)-2′,3′-dihydrospiro[cyclohexane-1,1′-isoindole]-4-carboxylate

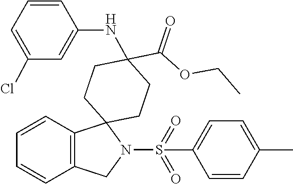 [SMILES: CCOC(=O)C1(Nc2cccc(Cl)c2)CCC2(CC1)c1ccccc1CN2S(=O)(=O)c1ccc(C)cc1]

Example 28G (320 mg, 0.6 mmol) was dissolved in DCM (20 mL). cc. H₂SO₄ (8 drops) was added to the mixture and stirred at 40° C. for 1 day. The mixture was diluted with sat. aq. NaHCO₃ solution, brine and extracted with DCM. The combined organic layers were dried over Na₂SO₄, filtered and the filtrate was concentrated under reduced pressure. The crude product was purified via flash chromatography using heptane and EtOAc as eluents to give Example 28H as a mixture of diastereoisomers. ¹H NMR (500 MHz, DMSO-d₆) δ ppm: 7.78/7.73 (d/d, 2H), 7.78/7.73 (d/d, 2H), 7.60-7.27 (m, 4H), 7.11/7.08 (t/t, 1H), 6.67/6.60 (dd/m, 1H), 6.63-6.47 (m, 2H), 6.39/6.36 (s/s, 1H), 4.63/4.56 (s/s, 2H), 4.21/4.16 (q/q, 2H), 2.93/2.71/1.73/1.53 (td+dm/td+br d, 4H), 2.56/2.28/2.21/2.00 (dm+td/td+br d, 4H), 2.37/2.35 (s/s, 3H), 1.23/1.14 (t/t, 3H). HRMS calculated for C₂₉H₃₁ClN₂O₄S: 538.1693; found 539.1773 and 539.1807 (M+H).

### Example 28 4-(3-chloroanilino)-2′-(4-methylbenzene-1-sulfonyl)-2′,3′-dihydrospiro[cyclohexane-1,1′-isoindole]-4-carboxylic acid, diastereoisomer 1

and

### Example 29 4-(3-chloroanilino)-2′-(4-methylbenzene-1-sulfonyl)-2′,3′-dihydrospiro[cyclohexane-1,1′-isoindole]-4-carboxylic acid, diastereoisomer 2

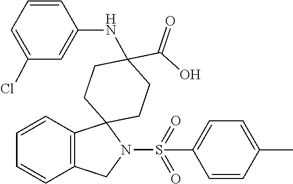 [SMILES: Cc1ccc(S(=O)(=O)N2Cc3ccccc3C23CCC(Nc2cccc(Cl)c2)(C(=O)O)CC3)cc1]

Using General procedure 33a and Example 28H as the appropriate ester a mixture of diastereoisomers was obtained. The diastereoisomers were separated by prep RP-HPLC using 25 mM aq. NH₄HCO₃ solution and MeCN as eluents. The diastereoisomer eluting earlier was collected as Example 28. ¹H NMR (500 MHz, DMSO-d₆) δ: 7.78 (m, 2H), 7.67-7.27 (m, 4H), 7.34 (m, 2H), 7.04 (t, 1H), 6.60 (t, 1H), 6.54 (dm, 1H), 6.51 (dm, 1H), 6.05 (br s, 1H), 4.55 (s, 2H), 2.86/1.48 (m+m, 4H), 2.34 (s, 3H), 2.27/2.11 (m+m, 4H). HRMS calculated for C₂₇H₂₇N₂O₄SCl: 510.1380; found: 511.1473 (M+H).

The diastereoisomer eluting later was collected as Example 29. ¹H NMR (500 MHz, DMSO-d₆) δ: 12.79 (br s, 1H), 7.74 (m, 2H), 7.58-7.26 (m, 4H), 7.40 (m, 2H), 7.06 (t, 1H), 6.71 (t, 1H), 6.63 (dm, 1H), 6.56 (dm, 1H), 6.28 (br s, 1H), 4.62 (s, 2H), 2.75/1.70 (m+m, 4H), 2.55/1.94 (m+m, 4H), 2.37 (s, 3H). HRMS calculated for C₂₇H₂₇N₂O₄SCl: 510.1380; found: 511.1471 (M+H).

### Example 30

### Example 30A 2-(3-methoxyphenyl)isoindolin-1-one

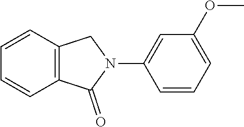 [SMILES: COc1cccc(N2Cc3ccccc3C2=O)c1]

Isoindolin-1-one (133 mg, 1.0 mmol), 1-bromo-3-methoxy-benzene (152 μL, 1.2 mmol), Pd₂(dba)₃ (46 mg, 0.05 mmol), XantPhos (87 mg, 0.15 mmol) and Cs₂CO₃ (488 mg, 1.5 mmol) were dissolved in 1,4-dioxane (5 mL) and stirred under N₂ at 100° C. for 3 h. The mixture was diluted with water, brine and extracted with DCM. The combined organic layers were dried over Na₂SO₄, filtered and the filtrate was concentrated under reduced pressure. The crude product was purified via flash chromatography using heptane and EtOAc as eluents to give Example 30A. ¹H NMR (500 MHz, DMSO-d₆) δ ppm: 7.78 (d, 1H), 7.69 (td, 1H), 7.66 (dm, 1H), 7.60 (t, 1H), 7.55 (td, 1H), 7.45 (dm, 1H), 7.35 (t, 1H), 6.77 (dm, 1H), 5.03 (s, 2H), 3.79 (s, 3H). HRMS calculated for C₁₅H₁₃NO₂: 239.0946; found 240.1021 (M+H).

### Example 30B 2″-(3-methoxyphenyl)dispiro[[1,3]dioxolane-2,1′-cyclohexane-4′,1″-isoindol]-3″(2″H)-one

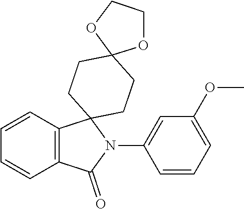 [SMILES: COc1cccc(N2C(=O)c3ccccc3C23CCC2(CC3)OCCO2)c1]

Using General procedure 8b and Example 30A as the appropriate isoindolin-1-one Example 30B was obtained. ¹H NMR (500 MHz, DMSO-d₆) δ ppm: 7.88 (dm, 1H), 7.79 (dm, 1H), 7.69 (m, 1H), 7.58 (m, 1H), 7.43 (t, 1H), 7.07 (dm, 1H), 6.84 (m, 1H), 6.84 (m, 1H), 3.88/3.80 (m+m, 4H), 3.79 (s, 3H), 2.16-1.65 (m, 8H). HRMS calculated for C₂₂H₂₃NO₄: 365.1627; found 366.1717 (M+H).

### Example 30C 2′-(3-methoxyphenyl)spiro[cyclohexane-4,3′-isoindoline]-1,1′-dione

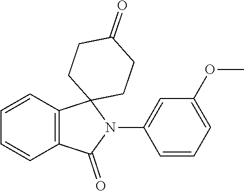 [SMILES: COc1cccc(N2C(=O)c3ccccc3C23CCC(=O)CC3)c1]

Using General procedure 9 and Example 30B as the appropriate ketal Example 30C was obtained. ¹H NMR (500 MHz, DMSO-d₆) δ ppm: 8.14 (dm, 1H), 7.82 (dm, 1H), 7.70 (m, 1H), 7.61 (m, 1H), 7.42 (t, 1H), 7.06 (dm, 1H), 6.90 (m, 1H), 6.89 (m, 1H), 3.78 (s, 3H), 2.85/2.29 (m+m, 4H), 2.31/2.10 (m+m, 4H). HRMS calculated for C₂₀H₁₉NO₃: 321.1365; found 322.1431 (M+H).

### Example 30D (1s,4s)-4-(3-bromoanilino)-2′-(3-methoxyphenyl)-3′-oxo-2′,3′-dihydrospiro[cyclohexane-1,1′-isoindole]-4-carbonitrile

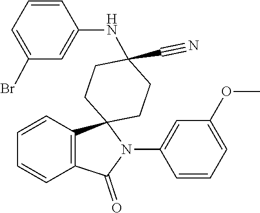 [SMILES: COc1cccc(N2C(=O)c3ccccc3[C@]23CC[C@](C#N)(Nc2cccc(Br)c2)CC3)c1]

Using General procedure 11 and Example 30C as the appropriate ketone and 3-bromoaniline as the appropriate aniline a mixture of diastereoisomers was obtained. The diastereoisomers were separated via flash chromatography using heptane and EtOAc as eluents. The diastereoisomer eluting earlier was collected as Example 30D. ¹H NMR (500 MHz, DMSO-d₆) δ ppm: 7.94 (dm, 1H), 7.78 (dm, 1H), 7.74 (m, 1H), 7.60 (m, 1H), 7.40 (t, 1H), 7.10 (t, 1H), 7.06 (dm, 1H), 6.90 (m, 1H), 6.88 (m, 1H), 6.87 (dm, 1H), 6.79 (t, 1H), 6.70 (dm, 1H), 6.24 (s, 1H), 3.77 (s, 3H), 2.44/2.16 (m+m, 4H), 2.24/1.83 (m+m, 4H). HRMS calculated for C₂₇H₂₄BrN₃O₂: 501.1052; found 502.1103 (M+H).

### Example 30E (1s,4s)-4-(3-bromoanilino)-2′-(3-methoxyphenyl)-3′-oxo-2′,3′-dihydrospiro[cyclohexane-1,1′-isoindole]-4-carboxamide

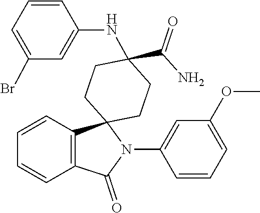 [SMILES: COc1cccc(N2C(=O)c3ccccc3[C@]23CC[C@@](Nc2cccc(Br)c2)(C(N)=O)CC3)c1]

Using General procedure 12b and Example 30D as the appropriate nitrile Example 30E was obtained. ¹H NMR (500 MHz, DMSO-d₆) δ ppm: 7.94 (dm, 1H), 7.79 (d, 1H), 7.73 (td, 1H), 7.59 (t, 1H), 7.40 (t, 1H), 7.06 (dm, 1H), 6.96 (t, 1H), 6.79 (dm, 1H), 6.78 (m, 1H), 6.71 (dm, 1H), 6.54 (t, 1H), 6.34 (dm, 1H), 5.92 (s, 1H), 3.78 (s, 3H), 2.47/2.04 (t+d, 4H), 2.10/1.48 (t+d, 4H). HRMS calculated for C₂₇H₂₆BrN₃O₃: 519.1157; found 520.1232 (M+H).

### Example 30 (1s,4s)-4-(3-bromoanilino)-2′-(3-methoxyphenyl)-3′-oxo-2′,3′-dihydrospiro[cyclohexane-1,1′-isoindole]-4-carboxylic acid

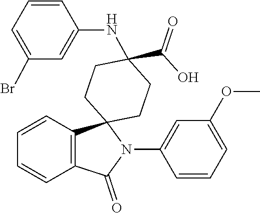 [SMILES: COc1cccc(N2C(=O)c3ccccc3[C@]23CC[C@@](Nc2cccc(Br)c2)(C(=O)O)CC3)c1]

Using General procedure 13 and Example 30E as the appropriate amide Example 30 was obtained. ¹H NMR (500 MHz, DMSO-d₆) δ: 7.90 (d, 1H), 7.80 (d, 1H), 7.73 (t, 1H), 7.59 (t, 1H), 7.39 (t, 1H), 7.05 (dm, 1H), 6.93 (t, 1H), 6.81 (dm, 1H), 6.80 (m, 1H), 6.64 (d, 1H), 6.53 (t, 1H), 6.36 (dd, 1H), 6.05 (br, 1H), 3.77 (s, 3H), 2.41/2.18 (t+d, 4H), 2.17/1.53 (t+d, 4H). HRMS calculated for C₂₇H₂₅N₂O₄Br: 520.0998; found: 521.1080 (M+H).

### Example 31

### Example 31A 2″-(3-methoxyphenyl)-2″,3″-dihydrodispiro[[1,3]dioxolane-2,1′-cyclohexane-4′,1″-isoindole]

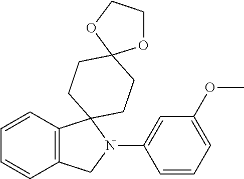 [SMILES: COc1cccc(N2Cc3ccccc3C23CCC2(CC3)OCCO2)c1]

Using General procedure 38 and Example 30B as the appropriate isoindolin-1-one Example 31A was obtained. ¹H NMR (500 MHz, DMSO-d₆) δ ppm: 7.70 (dd, 1H), 7.41 (dd, 1H), 7.35 (td, 1H), 7.32 (td, 1H), 7.11 (t, 1H), 6.68 (t, 1H), 6.57 (dd, 1H), 6.26 (dd, 1H), 4.60 (s, 2H), 4.03+3.97 (m+m. 4H), 3.78 (s, 3H), 3.05 (td, 2H), 2.13 (td, 2H), 1.82 (dd, 2H), 1.47 (dd, 2H).

### Example 31B 2′-(3-methoxyphenyl)spiro[cyclohexane-4,1′-isoindoline]-1-one

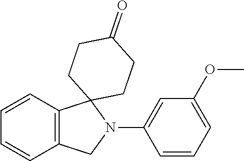 [SMILES: COc1cccc(N2Cc3ccccc3C23CCC(=O)CC3)c1]

Using General procedure 9 and Example 31A as the appropriate ketal Example 31B was obtained. ¹H NMR (400 MHz, DMSO-d₆) δ ppm: 7.45-7.30 (m, 4H), 7.17 (t, 1H), 6.52 (d, 1H), 6.36 (m, 1H), 6.34 (m, 1H), 4.60 (s, 2H), 3.76 (s, 3H), 3.00/1.83 (m+m, 4H), 2.64 (m, 4H). HRMS calculated for C₂₀H₂₁NO₂: 307.1572; found 308.1651 (M+H).

### Example 31 (1r,4r)-4-(3-bromoanilino)-2′-(3-methoxyphenyl)-2′,3′-dihydrospiro[cyclohexane-1,1′-isoindole]-4-carboxylic acid

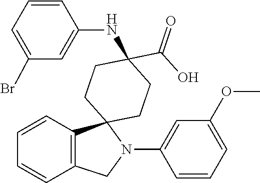 [SMILES: COc1cccc(N2Cc3ccccc3[C@]23CC[C@](Nc2cccc(Br)c2)(C(=O)O)CC3)c1]

Using General procedure 10 and Example 31B as the appropriate ketone and 3-bromoaniline as the appropriate aniline Example 31 was obtained. ¹H NMR (500 MHz, DMSO-d₆) δ ppm: 7.72 (dm, 1H), 7.41 (dm, 1H), 7.34 (m, 1H), 7.32 (m, 1H), 7.10 (t, 1H), 7.02 (t, 1H), 6.90 (t, 1H), 6.73 (dm, 1H), 6.72 (dm, 1H), 6.70 (dm, 1H), 6.53 (t, 1H), 6.36 (dm, 1H), 4.62 (s, 2H), 3.77 (s, 3H), 2.84/1.59 (m+m, 4H), 2.55/2.15 (m+m, 4H). HRMS calculated for C₂₇H₂₇BrN₂O₃: 506.1205; found 507.1277 (M+H).

### Example 32

### Example 32A 4-(3-bromoanilino)-2′-(3-methoxyphenyl)-2′,3′-dihydrospiro[cyclohexane-1,1′-isoindole]-4-carbonitrile

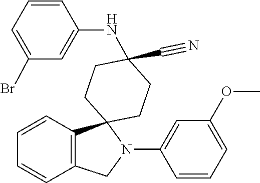 [SMILES: COc1cccc(N2Cc3ccccc3[C@]23CC[C@](C#N)(Nc2cccc(Br)c2)CC3)c1]

Using General procedure 11 and Example 31B as the appropriate ketone and 3-bromoaniline as the appropriate aniline Example 32A was obtained as a mixture of diastereoisomers. LRMS calculated for C₂₇H₂₆BrN₃O: 487.1259; found 488.2 (M+H).

### Example 32B (1s,4s)-4-(3-bromoanilino)-2′-(3-methoxyphenyl)-2′,3′-dihydrospiro[cyclohexane-1,1′-isoindole]-4-carboxamide

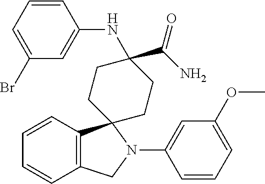 [SMILES: COc1cccc(N2Cc3ccccc3[C@]23CC[C@@](Nc2cccc(Br)c2)(C(N)=O)CC3)c1]

Using General procedure 12b and Example 32A as the appropriate nitrile a mixture of diastereoisomers was obtained. The diastereoisomers were separated via flash chromatography using heptane and EtOAc as eluents. The diastereoisomer eluting later was collected as Example 32B. ¹H NMR (500 MHz, DMSO-d₆) δ ppm: 7.75-7.33 (m, 4H), 7.26/7.18 (s+s, 2H), 7.12 (t, 1H), 7.10 (t, 1H), 7.01 (t, 1H), 6.78 (dm, 1H), 6.73 (dm, 1H), 6.67 (dm, 1H), 6.52 (t, 1H), 6.41 (s, 1H), 6.29 (dm, 1H), 4.60 (s, 2H), 3.77 (s, 3H), 3.03-1.20 (m, 8H). HRMS calculated for C₂₇H₂₈BrN₃O₂: 505.1365; found 506.1447 (M+H).

### Example 32 (1s,4s)-4-(3-bromoanilino)-2′-(3-methoxyphenyl)-2′,3′-dihydrospiro[cyclohexane-1,1′-isoindole]-4-carboxylic acid

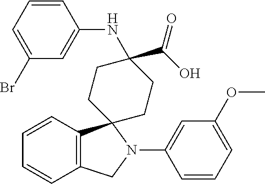 [SMILES: COc1cccc(N2Cc3ccccc3[C@]23CC[C@@](Nc2cccc(Br)c2)(C(=O)O)CC3)c1]

Using General procedure 13 and Example 32B as the appropriate amide Example 32 was obtained. ¹H NMR (500 MHz, DMSO-d₆) δ: 12.79 (br s, 1H), 7.69 (m, 1H), 7.45-7.33 (m, 3H), 7.08 (t, 1H), 7.06 (t, 1H), 6.97 (t, 1H), 6.76 (dm, 1H), 6.75 (dm, 1H), 6.67 (dm, 1H), 6.61 (s, 1H), 6.56 (t, 1H), 6.27 (dm, 1H), 4.60 (s, 2H), 3.75 (s, 3H), 2.98/1.32 (m+m, 4H), 2.37/2.31 (m+m, 4H). HRMS calculated for C₂₇H₂₇BrN₂O₃: 506.1205; found 507.1279 (M+H).

### Example 33

### Example 33A 2-(4-methoxyphenyl)isoindolin-1-one

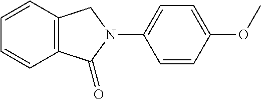 [SMILES: COc1ccc(N2Cc3ccccc3C2=O)cc1]

Isoindolin-1-one (999 mg, 7.5 mmol), 1-bromo-4-methoxy-benzene (1.13 mL, 9.0 mmol), Pd₂(dba)₃ (343 mg, 0.38 mmol), XantPhos (651 mg, 1.13 mmol) and Cs₂CO₃ (3.67 g, 11.25 mmol) were dissolved in 1,4-dioxane (37 mL) and stirred under N₂ at 100° C. for 17 h. The mixture was diluted with water, brine and extracted with DCM. The combined organic layers were dried over Na₂SO₄, filtered and the filtrate was concentrated under reduced pressure. The crude product was purified via flash chromatography using heptane and EtOAc as eluents to give Example 33A. ¹H NMR (500 MHz, DMSO-d₆) δ ppm: 7.8 (m, 2H), 7.77 (m, 1H), 7.66 (m, 1H), 7.66 (m, 1H), 7.53 (m, 1H), 7.02 (m, 2H), 4.98 (s, 2H), 3.77 (s, 3H). HRMS calculated for C₁₅H₁₃NO₂: 239.0946; found 240.1030 (M+H).

### Example 33B 2″-(4-methoxyphenyl)dispiro[[1,3]dioxolane-2,1′-cyclohexane-4′,1″-isoindol]-3″(2″H)-one

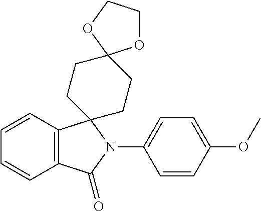 [SMILES: COc1ccc(N2C(=O)c3ccccc3C23CCC2(CC3)OCCO2)cc1]

Using General procedure 8b and Example 33A as the appropriate isoindolin-1-one Example 33B was obtained. ¹H NMR (500 MHz, DMSO-d₆) δ ppm: 7.87 (d, 1H), 7.78 (d, 1H), 7.68 (td, 1H), 7.57 (t, 1H), 7.17 (dm, 2H), 7.05 (dm, 2H), 3.87+3.80 (m+m, 4H), 3.83 (s, 3H), 2.16-1.61 (m, 8H). LRMS calculated for C₂₂H₂₃NO₄: 365.1627; found 366.2 (M+H).

### Example 33C 2′-(4-methoxyphenyl)spiro[cyclohexane-4,3′-isoindoline]-1,1′-dione

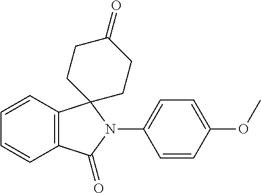 [SMILES: COc1ccc(N2C(=O)c3ccccc3C23CCC(=O)CC3)cc1]

Using General procedure 9 and Example 33B as the appropriate ketal Example 33C was obtained. ¹H NMR (500 MHz, DMSO-d₆) δ ppm: 8.13 (d, 1H), 7.81 (d, 1H), 7.69 (t, 1H), 7.60 (t, 1H), 7.23 (m, 2H), 7.05 (m, 2H), 3.81 (s, 3H), 2.85/2.28 (m, 4H), 2.25/2.06 (m, 4H). HRMS calculated for C₂₀H₁₉NO₃: 321.1365; found 322.1443 (M+H).

### Example 33D (1s,4s)-4-(3-bromoanilino)-2′-(4-methoxyphenyl)-3′-oxo-2′,3′-dihydrospiro[cyclohexane-1,1′-isoindole]-4-carbonitrile

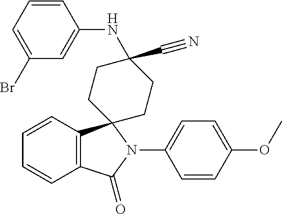 [SMILES: COc1ccc(N2C(=O)c3ccccc3[C@]23CC[C@](C#N)(Nc2cccc(Br)c2)CC3)cc1]

Using General procedure 11 and Example 33C as the appropriate ketone and 3-bromoaniline as the appropriate aniline a mixture of diastereoisomers was obtained. The diastereoisomers were separated via flash chromatography using heptane and EtOAc as eluents. The diastereoisomer eluting earlier was collected as Example 33D. ¹H NMR (500 MHz, DMSO-d₆) δ ppm: 7.93 (dm, 1H), 7.77 (dm, 1H), 7.73 (m, 1H), 7.59 (m, 1H), 7.21 (m, 2H), 7.10 (t, 1H), 7.03 (m, 2H), 6.88 (dm, 1H), 6.78 (t, 1H), 6.72 (dm, 1H), 6.21 (s, 1H), 3.82 (s, 3H), 2.43/2.15 (m+m, 4H), 2.20/1.81 (m+m, 4H). HRMS calculated for C₂₇H₂₄BrN₃O₂: 501.1052; found 502.1128 (M+H).

### Example 33E (1s,4s)-4-(3-bromoanilino)-2′-(4-methoxyphenyl)-3′-oxo-2′,3′-dihydrospiro[cyclohexane-1,1′-isoindole]-4-carboxamide

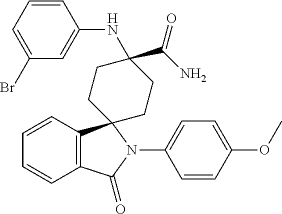 [SMILES: COc1ccc(N2C(=O)c3ccccc3[C@]23CC[C@@](Nc2cccc(Br)c2)(C(N)=O)CC3)cc1]

Using General procedure 12b and Example 33D as the appropriate nitrile Example 33E was obtained. ¹H NMR (500 MHz, DMSO-d₆) δ ppm: 7.93 (dm, 1H), 7.78 (dm, 1H), 7.72 (m, 1H), 7.58 (m, 1H), 7.27/7.25 (d+d, 2H), 7.13 (m, 2H), 7.03 (m, 2H), 6.97 (t, 1H), 6.71 (dm, 1H), 6.54 (t, 1H), 6.35 (dm, 1H), 5.87 (s, 1H), 3.84 (s, 3H), 2.46/2.04 (m+m, 4H), 2.06/1.45 (m+m, 4H). HRMS calculated for C₂₇H₂₆BrN₃O₃: 519.1157; found 520.1246 (M+H).

### Example 33 (1s,4s)-4-(3-bromoanilino)-2′-(4-methoxyphenyl)-3′-oxo-2′,3′-dihydrospiro[cyclohexane-1,1′-isoindole]-4-carboxylic acid

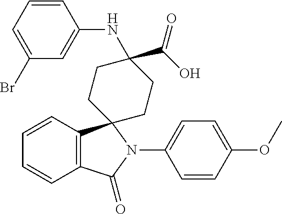 [SMILES: COc1ccc(N2C(=O)c3ccccc3[C@]23CC[C@@](Nc2cccc(Br)c2)(C(=O)O)CC3)cc1]

Using General procedure 13 and Example 33E as the appropriate amide Example 33 was obtained. ¹H NMR (500 MHz, DMSO-d₆) δ: 7.89 (d, 1H), 7.79 (d, 1H), 7.73 (t, 1H), 7.59 (t, 1H), 7.14 (dm, 2H), 7.03 (dm, 2H), 6.94 (t, 1H), 6.66 (dd, 1H), 6.54 (t, 1H), 6.36 (dd, 1H), 6.02 (br, 1H), 3.82 (s, 3H), 2.41/2.18 (t+d, 4H), 2.13/1.51 (t+d, 4H). HRMS calculated for C₂₇H₂₅BrN₂O₄: 520.0998; found 521.1062 (M+H).

### Example 34

### Example 34A 2-(3-phenoxypropyl)isoindolin-1-one

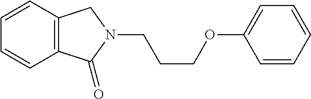 [SMILES: O=C1c2ccccc2CN1CCCOc1ccccc1]

Using General procedure 37 and isoindolin-1-one as the appropriate isoindolin-1-one and 3-bromopropoxybenzene as the appropriate alkyl bromide Example 34A was obtained. ¹H NMR (500 MHz, DMSO-d₆) δ ppm: 7.70-7.43 (m, 4H), 7.26 (m, 2H), 6.91 (tm, 1H), 6.89 (dm, 2H), 4.51 (s, 2H), 4.00 (t, 2H), 3.69 (t, 2H), 2.07 (quint, 2H). HRMS calculated for C₁₇H₁₇NO₂: 267.1259; found 268.1340 (M+H).

### Example 34B 2″-(3-phenoxypropyl)dispiro[[1,3]dioxolane-2,1′-cyclohexane-4′,1″-isoindol]-3″(2″H)-one

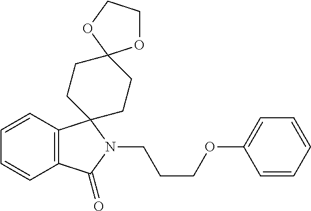 [SMILES: O=C1c2ccccc2C2(CCC3(CC2)OCCO3)N1CCCOc1ccccc1]

Using General procedure 8b and Example 34A as the appropriate isoindolin-1-one Example 34B was obtained. ¹H NMR (500 MHz, DMSO-d₆) δ ppm: 7.84 (dm, 1H), 7.71 (dm, 1H), 7.60 (m, 1H), 7.52 (m, 1H), 7.29 (m, 2H), 6.95 (m, 2H), 6.93 (m, 1H), 4.03 (t, 2H), 4.01-3.93 (m, 4H), 3.51 (m, 2H), 2.28/1.38 (m+m, 4H), 2.11 (m, 2H), 2.11/1.83 (m+m, 4H). HRMS calculated for C₂₄H₂₇NO₄: 393.194; found 394.2020 (M+H).

### Example 34C 2′-(3-phenoxypropyl)spiro[cyclohexane-4,3′-isoindoline]-1,1′-dione

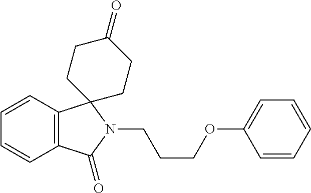 [SMILES: O=C1CCC2(CC1)c1ccccc1C(=O)N2CCCOc1ccccc1]

Using General procedure 9 and Example 34B as the appropriate ketal Example 34C was obtained. ¹H NMR (500 MHz, DMSO-d₆) δ ppm: 8.10 (d, 1H), 7.74 (d, 1H), 7.61 (td, 1H), 7.55 (td, 1H), 7.28 (m, 2H), 6.93 (m, 2H), 6.92 (m, 1H), 4.04 (t, 2H), 3.61 (t, 2H), 2.93/2.43 (m, 4H), 2.56/1.71 (m, 4H), 2.10 (quint, 2H). HRMS calculated for C₂₂H₂₃NO₃: 349.1678; found 350.1745 (M+H).

### Example 34D (1s,4s)-4-(3-chloroanilino)-3′-oxo-2′-(3-phenoxypropyl)-2′,3′-dihydrospiro[cyclohexane-1,1′-isoindole]-4-carbonitrile

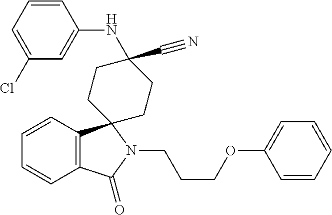 [SMILES: N#C[C@]1(Nc2cccc(Cl)c2)CC[C@@]2(CC1)c1ccccc1C(=O)N2CCCOc1ccccc1]

Using General procedure 11 and Example 34C as the appropriate ketone and 3-chloroaniline as the appropriate aniline a mixture of diastereoisomers was obtained. The diastereoisomers were separated via via flash chromatography using heptane and EtOAc as eluents. The diastereoisomer eluting earlier was collected as Example 34D. ¹H NMR (500 MHz, DMSO-d₆) δ ppm: 8.04 (d, 1H), 7.71 (d, 1H), 7.64 (td, 1H), 7.54 (t, 1H), 7.28 (t, 1H), 7.22 (m, 2H), 7.02 (t, 1H), 6.95 (dm, 1H), 6.90 (tm, 1H), 6.84 (dm, 1H), 6.80 (dm, 2H), 6.62 (s, 1H), 3.98 (t, 2H), 3.51 (m, 2H), 2.56/2.49 (d+td, 4H), 2.33/1.35 (td+d, 4H), 2.11 (m, 2H). HRMS calculated for C₂₉H₂₈ClN₃O₂: 485.187; found 486.1948 (M+H).

### Example 34E (1s,4s)-4-(3-chloroanilino)-3′-oxo-2′-(3-phenoxypropyl)-2′,3′-dihydrospiro[cyclohexane-1,1′-isoindole]-4-carboxamide

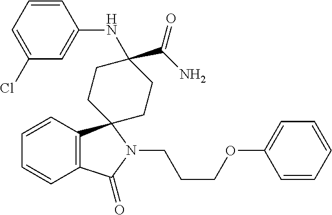 [SMILES: NC(=O)[C@]1(Nc2cccc(Cl)c2)CC[C@@]2(CC1)c1ccccc1C(=O)N2CCCOc1ccccc1]

Using General procedure 12b and Example 34D as the appropriate nitrile Example 34E was obtained. ¹H NMR (500 MHz, DMSO-d₆) δ ppm: 7.86-7.50 (m, 4H), 7.50/7.27 (s+s, 2H), 7.23 (m, 2H), 7.14 (t, 1H), 6.90 (m, 1H), 6.85 (m, 2H), 6.79 (t, 1H), 6.66 (dm, 1H), 6.64 (dm, 1H), 6.32 (s, 1H), 4.00 (t, 2H), 3.56 (m, 2H), 2.42/2.32 (m+m, 4H), 2.11/1.18 (m+m, 4H), 2.11 (m, 2H). HRMS calculated for C₂₉H₃₀ClN₃O₃: 503.1976; found 504.2059 (M+H).

### Example 34 (1s,4s)-4-(3-chloroanilino)-3′-oxo-2′-(3-phenoxypropyl)-2′,3′-dihydrospiro[cyclohexane-1,1′-isoindole]-4-carboxylic acid

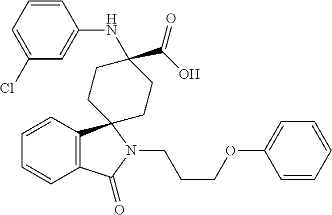 [SMILES: O=C1c2ccccc2[C@]2(CC[C@@](Nc3cccc(Cl)c3)(C(=O)O)CC2)N1CCCOc1ccccc1]

Using General procedure 13 and Example 34E as the appropriate amide Example 34 was obtained. ¹H NMR (500 MHz, DMSO-d₆) δ: 7.92-7.46 (m, 4H), 7.21 (m, 2H), 7.03 (t, 1H), 6.88 (m, 1H), 6.83 (m, 2H), 6.71 (t, 1H), 6.62 (dm, 1H), 6.49 (dm, 1H), 6.29 (br s, 1H), 3.98 (t, 2H), 3.53 (m, 2H), 2.35/2.17 (m+m, 4H), 2.35/1.13 (m+m, 4H), 2.11 (m, 2H). HRMS calculated for C₂₉H₂₉ClN₂O₄: 504.1816; found 505.1879 (M+H).

### Example 35

### Example 35A 2″-(3-phenoxypropyl)-2″,3″-dihydrodispiro[[1,3]dioxolane-2,1′-cyclohexane-4′,1″-isoindole]

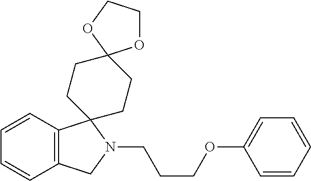 [SMILES: c1ccc(OCCCN2Cc3ccccc3C23CCC2(CC3)OCCO2)cc1]

Using General procedure 38 and Example 34B as the appropriate ketal Example 35A was obtained. ¹H NMR (500 MHz, DMSO-d₆) δ ppm: 7.51-7.16 (m, 4H), 7.26 (m, 2H), 6.92 (m, 2H), 6.90 (m, 1H), 4.03 (t, 2H), 3.95-3.85 (m, 4H), 3.91 (s, 2H), 2.76 (t, 2H), 1.91/1.45 (m+m, 4H), 1.91/1.71 (m+m, 4H), 1.90 (m, 2H). HRMS calculated for C₂₄H₂₉NO₃: 379.2148; found 380.2229 (M+H).

### Example 35B 2′-(3-phenoxypropyl)spiro[cyclohexane-4,1′-isoindoline]-1-one

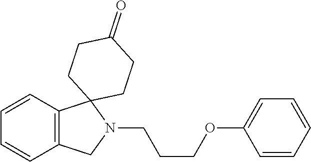 [SMILES: O=C1CCC2(CC1)c1ccccc1CN2CCCOc1ccccc1]

Using General procedure 9 and Example 35A as the appropriate ketal Example 35B was obtained. ¹H NMR (500 MHz, DMSO-d₆) δ ppm: 7.54 (d, 1H), 7.29 (d, 1H), 7.27-7.18 (m, 2H), 7.26 (m, 2H), 6.91 (m, 2H), 6.90 (m, 1H), 4.05 (t, 2H), 3.95 (s, 2H), 2.84 (t, 2H), 2.61/2.38 (m, 4H), 2.13/1.76 (m, 4H), 1.93 (quint, 2H). HRMS calculated for C₂₂H₂₅NO₂: 335.1885; found 336.1970 (M+H).

### Example 35C 4-(3-chloroanilino)-2′-(3-phenoxypropyl)-2′,3′-dihydrospiro[cyclohexane-1,1′-isoindole]-4-carbonitrile

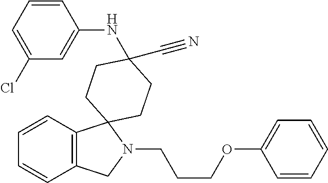 [SMILES: N#CC1(Nc2cccc(Cl)c2)CCC2(CC1)c1ccccc1CN2CCCOc1ccccc1]

Using General procedure 11 and Example 35B as the appropriate ketone and 3-chloroaniline as the appropriate aniline Example 35C was obtained as a mixture of diastereoisomers. ¹H NMR (500 MHz, DMSO-d₆) δ ppm: 7.58-6.74 (m, 13H), 6.48/6.45 (s, 1H), 4.06/4.01 (t, 2H), 3.96/3.93 (s, 2H), 2.87/2.76 (t, 2H), 2.46-1.37 (m, 10H). HRMS calculated for C₂₉H₃₀ClN₃O: 471.2077; found 472.2150 and 472.2145 (M+H).

### Example 35D (1s,4s)-4-(3-chloroanilino)-2′-(3-phenoxypropyl)-2′,3′-dihydrospiro[cyclohexane-1,1′-isoindole]-4-carboxamide

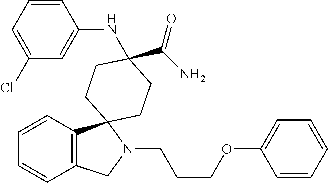 [SMILES: NC(=O)[C@]1(Nc2cccc(Cl)c2)CC[C@@]2(CC1)c1ccccc1CN2CCCOc1ccccc1]

Using General procedure 12a and Example 35C as the appropriate nitrile a mixture of diastereoisomers was obtained. The diastereoisomers were separated via via flash chromatography using heptane and EtOAc as eluents. The diastereoisomer eluting later was collected as Example 35D. ¹H NMR (500 MHz, DMSO-d₆) δ ppm: 7.54 (m, 1H), 7.33/7.15 (br+br, 2H), 7.26 (m, 1H), 7.22 (m, 2H), 7.21 (m, 2H), 7.10 (t, 1H), 6.87 (tm, 1H), 6.85 (dm, 2H), 6.72 (t, 1H), 6.62 (dm, 1H), 6.59 (dm, 1H), 6.10 (s, 1H), 4.02 (t, 2H), 3.91 (s, 2H), 2.84 (t, 2H), 2.25/1.97 (td+d, 4H), 1.95/1.24 (t+d, 4H), 1.91 (m, 2H). HRMS calculated for C₂₉H₃₂ClN₃O₂: 489.2183; found 490.2269 (M+H).

### Example 35 (1s,4s)-4-(3-chloroanilino)-2′-(3-phenoxypropyl)-2′,3′-dihydrospiro[cyclohexane-1,1′-isoindole]-4-carboxylic acid

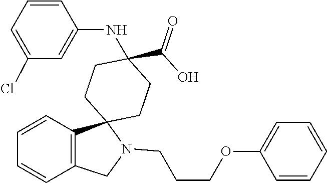 [SMILES: O=C(O)[C@]1(Nc2cccc(Cl)c2)CC[C@@]2(CC1)c1ccccc1CN2CCCOc1ccccc1]

Using General procedure 13 and Example 35D as the appropriate amide Example 35 was obtained. ¹H NMR (500 MHz, DMSO-d₆) δ: 12.71 (br s, 1H), 7.51 (m, 1H), 7.27 (m, 1H), 7.23 (m, 2H), 7.20 (m, 2H), 7.08 (t, 1H), 6.87 (tm, 1H), 6.85 (dm, 2H), 6.63 (t, 1H), 6.57 (dm, 1H), 6.53 (dm, 1H), 6.29 (br, 1H), 4.01 (t, 2H), 3.92 (s, 2H), 2.81 (t, 2H), 2.19/2.11 (td+d, 4H), 2.00/1.28 (td+d, 4H), 1.9 (m, 2H). HRMS calculated for C₂₉H₃₁ClN₂O₃: 490.2023; found 491.2102 (M+H).

### Example 36

### Example 36A 2-[3-[tert-butyl(dimethyl)silyl]oxypropyl]isoindolin-1-one

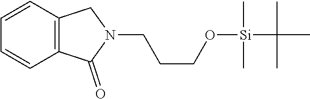 [SMILES: CC(C)(C)[Si](C)(C)OCCCN1Cc2ccccc2C1=O]

Using General procedure 37 and isoindolin-1-one as the appropriate isoindolin-1-one and 3-bromopropoxy-tert-butyl-dimethyl-silane as the appropriate alkyl bromide Example 36A was obtained. ¹H NMR (500 MHz, DMSO-d₆) δ ppm: 7.66 (d, 1H), 7.58 (m, 2H), 7.47 (m, 1H), 4.47 (s, 2H), 3.64 (t, 2H), 3.57 (t, 2H), 1.79 (quint, 2H), 0.85 (s, 9H), 0.01 (s, 6H). HRMS calculated for C₁₇H₂₇NO₂Si: 305.1811; found 306.1883 (M+H).

### Example 36B 2″-(3-{[tert-butyl(dimethyl)silyl]oxy}propyl)dispiro[[1,3]dioxolane-2,1′-cyclohexane-4′,1″-isoindol]-3″(2″H)-one

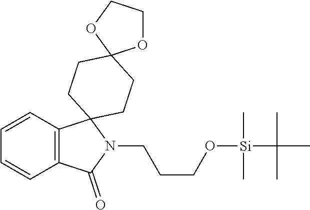 [SMILES: CC(C)(C)[Si](C)(C)OCCCN1C(=O)c2ccccc2C12CCC1(CC2)OCCO1]

Using General procedure 8b and Example 36A as the appropriate isoindolin-1-one Example 36B was obtained. ¹H NMR (500 MHz, DMSO-d₆) δ ppm: 7.84 (d, 1H), 7.69 (d, 1H), 7.60 (t, 1H), 7.51 (t, 1H), 3.94 (t, 2H), 3.94 (t, 2H), 3.65 (t, 2H), 3.40 (t, 2H), 2.28/1.37 (t+d, 4H), 2.11/1.84 (t+d, 4H), 1.84 (m, 2H), 0.91 (s, 9H), 0.06 (s, 6H). HRMS calculated for C₂₄H₃₇NO₄Si: 431.2492; found 432.2569 (M+H).

### Example 36C 2″-(3-{[tert-butyl(dimethyl)silyl]oxy}propyl)-2″,3″-dihydrodispiro[[1,3]dioxolane-2,1′-cyclohexane-4′,1″-isoindole]

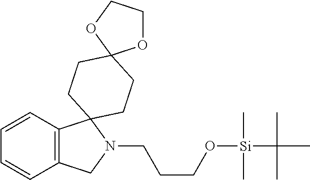 [SMILES: CC(C)(C)[Si](C)(C)OCCCN1Cc2ccccc2C12CCC1(CC2)OCCO1]

Using General procedure 38 and Example 36B as the appropriate ketal Example 36C was obtained. ¹H NMR (500 MHz, DMSO-d₆) δ ppm: 7.47 (m, 1H), 7.26 (m, 1H), 7.21 (m, 1H), 7.19 (m, 1H), 3.89 (s, 2H), 3.89 (s, 2H), 3.86 (s, 2H), 3.65 (t, 2H), 2.67 (t, 2H), 1.94/1.45 (t+d, 4H), 1.90/1.72 (t+d, 4H), 1.63 (quint, 2H), 0.88 (s, 9H), 0.03 (s, 6H). HRMS calculated for C₂₄H₃₉NO₃Si: 417.2699; found 418.2778 (M+H).

### Example 36D 2′-(3-hydroxypropyl)spiro[cyclohexane-4,1′-isoindoline]-1-one

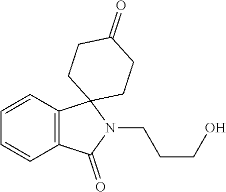 [SMILES: O=C1CCC2(CC1)c1ccccc1C(=O)N2CCCO]

Using General procedure 9 and Example 36C as the appropriate ketal Example 36D was obtained. ¹H NMR (500 MHz, DMSO-d₆) δ ppm: 7.56 (dm, 1H), 7.29 (dm, 1H), 7.26-7.19 (m, 2H), 4.43 (br, 1H), 3.91 (s, 2H), 3.49 (br t, 2H), 2.73 (t, 2H), 2.64/2.39 (m+dm, 4H), 2.14/1.79 (m+dm, 4H), 1.63 (m, 2H). HRMS calculated for C₁₆H₂₁NO₂: 259.1572; found 260.1635 (M+H).

### Example 36E (1s,4s)-4-(3-chloroanilino)-2′-(3-hydroxypropyl)-2′,3′-dihydrospiro[cyclohexane-1,1′-isoindole]-4-carbonitrile

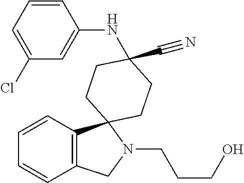

Using General procedure 11 and Example 36D as the appropriate ketone and 3-chloroaniline as the appropriate aniline a mixture of diastereoisomers was obtained. The diastereoisomers were separated via flash chromatography using heptane and EtOAc as eluents. The diastereoisomer eluting later was collected as Example 36E. ¹H NMR (500 MHz, DMSO-d₆) δ ppm: 7.53 (m, 1H), 7.31-7.19 (m, 3H), 7.22 (t, 1H), 6.92 (t, 1H), 6.87 (dm, 1H), 6.77 (dm, 1H), 6.46 (s, 1H), 4.42 (br, 1H), 3.88 (s, 2H), 3.48 (br, 2H), 2.63 (t, 2H), 2.39-2.26 (m, 4H), 2.00/1.47 (m+dm, 4H), 1.61 (m, 2H). HRMS calculated for C₂₃H₂₆ClN₃O: 395.1765; found 396.1828 (M+H).

### Example 36F (1s,4s)-4-(3-chloroanilino)-2′-(3-hydroxypropyl)-2′,3′-dihydrospiro[cyclohexane-1,1′-isoindole]-4-carboxamide

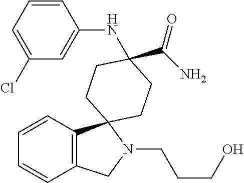 [SMILES: NC(=O)[C@]1(Nc2cccc(Cl)c2)CC[C@@]2(CC1)c1ccccc1CN2CCCO]

Using General procedure 12a and Example 36E as the appropriate nitrile Example 36F was obtained. ¹H NMR (500 MHz, DMSO-d₆) δ ppm: 7.53 (m, 1H), 7.34/7.15 (br s+br s, 2H), 7.30-7.19 (m, 3H), 7.09 (t, 1H), 6.69 (t, 1H), 6.60 (dm, 1H), 6.58 (dm, 1H), 6.12 (s, 1H), 4.46 (br s, 1H), 3.87 (s, 2H), 3.50 (t, 2H), 2.72 (t, 2H), 2.25/1.98 (m+m, 4H), 1.94/1.27 (m+m, 4H), 1.63 (m, 2H). HRMS calculated for C₂₃H₂₈ClN₃O₂: 413.187; found 414.1947 (M+H).

### Example 36G (1s,4s)-4-(3-chloroanilino)-2′-(3-hydroxypropyl)-2′,3′-dihydrospiro[cyclohexane-1,1′-isoindole]-4-carboxylic acid

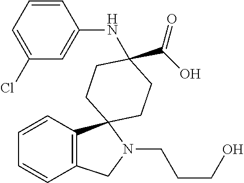 [SMILES: O=C(O)[C@]1(Nc2cccc(Cl)c2)CC[C@@]2(CC1)c1ccccc1CN2CCCO]

Using General procedure 13 and Example 36F as the appropriate amide Example 36G was obtained. ¹H NMR (500 MHz, DMSO-d₆) δ ppm: 7.53 (m, 1H), 7.31-7.17 (m, 3H), 7.04 (t, 1H), 6.60 (t, 1H), 6.53 (m, 2H), 6.24 (br, 1H), 3.88 (s, 2H), 3.49 (t, 2H), 2.69 (t, 2H), 2.21/2.11 (td+d, 4H), 1.98/1.30 (td+d, 4H), 1.63 (quint, 2H). HRMS calculated for C₂₃H₂₇ClN₂O₃: 414.171; found 415.1788 (M+H).

### Example 36H methyl (1s,4s)-4-(3-chloroanilino)-2′-(3-hydroxypropyl)-2′,3′-dihydrospiro[cyclohexane-1,1′-isoindole]-4-carboxylate

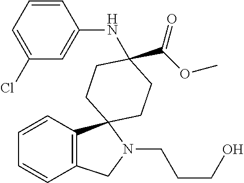 [SMILES: COC(=O)[C@]1(Nc2cccc(Cl)c2)CC[C@@]2(CC1)c1ccccc1CN2CCCO]

Using General procedure 17a and Example 36G as the appropriate amino acid Example 36H was obtained. ¹H NMR (500 MHz, DMSO-d₆) δ ppm: 7.51 (m, 1H), 7.32-7.19 (m, 3H), 7.07 (t, 1H), 6.60 (t, 1H), 6.58 (dm, 1H), 6.44 (dm, 1H), 6.38 (s, 1H), 4.45 (t, 1H), 3.88 (s, 2H), 3.65 (s, 3H), 3.49 (m, 2H), 2.69 (t, 2H), 2.23/2.15 (m+m, 4H), 2.00/1.32 (m+m, 4H), 1.63 (m, 2H). HRMS calculated for C₂₄H₂₉ClN₂O₃: 428.1867; found 429.1932 (M+H).

### Example 36 (1s,4s)-4-(3-chloroanilino)-2′-{3-[(thieno[3,2-b]pyridin-7-yl)oxy]propyl}-2′,3′-dihydrospiro[cyclohexane-1,1′-isoindole]-4-carboxylic acid

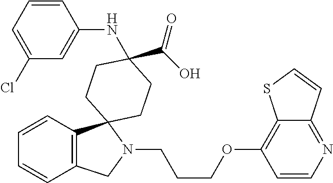 [SMILES: O=C(O)[C@]1(Nc2cccc(Cl)c2)CC[C@@]2(CC1)c1ccccc1CN2CCCOc1ccnc2ccsc12]

Using General procedure 32 and Example 36H as the appropriate isoindoline and thieno[3,2-b]pyridin-7-ol as the appropriate alcohol Example 36 was obtained. ¹H NMR (500 MHz, DMSO-d₆) δ: 8.46 (d, 1H), 7.79 (d, 1H), 7.53 (m, 1H), 7.45 (d, 1H), 7.27 (m, 1H), 7.24-7.18 (m, 2H), 7.03 (t, 1H), 6.94 (d, 1H), 6.61 (t, 1H), 6.53 (m, 2H), 6.13 (br, 1H), 4.33 (t, 2H), 3.94 (s, 2H), 2.87 (t, 2H), 2.18/2.02 (td+m, 4H), 2.02 (quint, 2H), 2.02/1.24 (m+d, 4H). HRMS calculated for C₃₀H₃₀ClN₃O₃S: 547.1696; found 548.1786 (M+H).

### Example 37 (1s,4s)-4-(3-chloroanilino)-2′-{3-[(3-methylpyridin-4-yl)oxy]propyl}-2′,3′-dihydrospiro[cyclohexane-1,1′-isoindole]-4-carboxylic acid

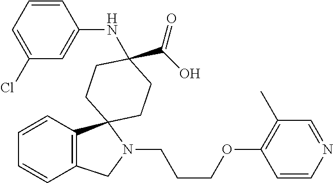 [SMILES: Cc1cnccc1OCCCN1Cc2ccccc2[C@]12CC[C@@](Nc1cccc(Cl)c1)(C(=O)O)CC2]

Using General procedure 32 and Example 36H as the appropriate isoindoline and 3-methylpyridin-4-ol as the appropriate alcohol Example 37 was obtained. ¹H NMR (500 MHz, DMSO-d₆) δ: 12.78 (br s, 1H), 8.22 (d, 1H), 8.10 (br s, 1H), 7.52 (m, 1H), 7.31-7.19 (m, 3H), 7.05 (t, 1H), 6.91 (d, 1H), 6.60 (t, 1H), 6.54 (dm, 1H), 6.49 (dm, 1H), 6.22 (br s, 1H), 4.11 (t, 2H), 3.93 (s, 2H), 2.82 (t, 2H), 2.19/2.08 (m+m, 4H), 1.98 (s, 3H), 1.97/1.27 (m+m, 4H), 1.95 (m, 2H). HRMS calculated for C₂₉H₃₂ClN₃O₃: 505.2132; found 506.2200 (M+H).

### Example 38

### Example 38A 2-[(2R)-3-[(4-methoxyphenyl)methoxy]-2-methyl-propyl]isoindolin-1-one

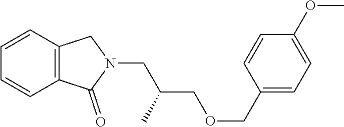 [SMILES: COc1ccc(COC[C@H](C)CN2Cc3ccccc3C2=O)cc1]

Using General procedure 37 and isoindolin-1-one as the appropriate isoindolin-1-one and Preparation 3c as the appropriate alkyl bromide Example 38A was obtained. ¹H NMR (500 MHz, DMSO-d₆) δ ppm: 7.68-7.46 (m, 4H), 7.22/6.87 (m+m, 4H), 4.43 (s, 2H), 4.36 (s, 2H), 3.73 (s, 3H), 3.46 (m, 2H), 3.30 (m, 2H), 2.18 (m, 1H), 0.86 (d, 3H). HRMS calculated for C₂₀H₂₃NO₃: 325.1678; found 326.1743 (M+H).

Example 38B 2-[(2R)-3-hydroxy-2-methyl-propyl]isoindolin-1-one

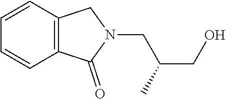

Using General procedure 28a and Example 38A as the appropriate PMB derivative Example 38B was obtained. ¹H NMR (500 MHz, DMSO-d₆) δ ppm: 7.67 (m, 1H), 7.59 (m, 2H), 7.48 (m, 1H), 4.56 (t, 1H), 4.48 (d, 2H), 3.43 (d, 2H), 3.30 (m, 2H), 1.98 (m, 1H), 0.83 (d, 3H). HRMS calculated for Cl₂H₁₅NO₂: 205.1103; found 206.1183 (M+H).

### Example 38C 2-[(2R)-3-[tert-butyl(dimethyl)silyl]oxy-2-methyl-propyl]isoindolin-1-one

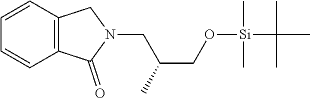 [SMILES: C[C@@H](CO[Si](C)(C)C(C)(C)C)CN1Cc2ccccc2C1=O]

Example 38B (3.4 g, 18.7 mmol) was dissolved in dry DCM (100 mL). DBU (4.2 mL, 28.1 mmol) and TBDMS-Cl (4.2 g, 18.7 mmol) were added to the mixture and stirred at rt until no further conversion was observed. The reaction mixture was diluted with sat. aq. NaHCO₃ solution, brine and extracted with DCM. The combined organic layers were washed with brine, dried over Na₂SO₄, filtered and the filtrate was concentrated under reduced pressure. The crude product was purified via flash chromatography using heptane and EtOAc as eluents to give Example 38C. ¹H NMR (500 MHz, DMSO-d₆) δ ppm: 7.66 (d, 1H), 7.58 (m, 2H), 7.47 (m, 1H), 4.46 (d, 2H), 3.50 (t, 2H), 3.43 (t, 2H), 2.05 (q, 1H), 0.86 (s, 9H), 0.84 (d, 3H), 0.01 (d, 6H). HRMS calculated for C₁₈H₂₉NO₂Si: 319.1967; found 320.2034 (M+H).

### Example 38D 2″-[(2R)-3-{[tert-butyl(dimethyl)silyl]oxy}-2-methylpropyl]dispiro[[1,3]dioxolane-2,1′-cyclohexane-4′,1″-isoindol]-3″(2″H)-one

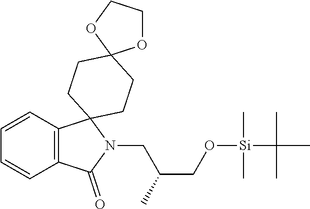 [SMILES: C[C@@H](CO[Si](C)(C)C(C)(C)C)CN1C(=O)c2ccccc2C12CCC1(CC2)OCCO1]

Using General procedure and Example 38C as the appropriate isoindolin-1-one Example 38D was obtained. ¹H NMR (500 MHz, DMSO-d₆) δ ppm: 7.84 (d, 1H), 7.69 (d, 1H), 7.59 (td, 1H), 7.51 (t, 1H), 3.99-3.89 (m, 4H), 3.50/3.47 (dd+dd, 2H), 3.30/3.20 (dd+dd, 2H), 2.40/1.25 (m, 8H), 2.36 (m, 1H), 0.89 (s, 9H), 0.86 (d, 3H), 0.03 (s, 6H). HRMS calculated for C₂₅H₃₉NO₄Si: 445.2648; found 446.2731 (M+H).

### Example 38E 2″-[(2R)-3-{[tert-butyl(dimethyl)silyl]oxy}-2-methylpropyl]-2″,3″-dihydrodispiro[[1,3]dioxolane-2,1′-cyclohexane-4′,1″-isoindole]

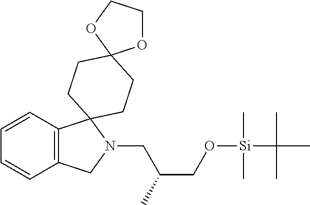 [SMILES: C[C@@H](CO[Si](C)(C)C(C)(C)C)CN1Cc2ccccc2C12CCC1(CC2)OCCO1]

Using General procedure 38 and Example 38D as the appropriate ketal Example 38E was obtained. ¹H NMR (500 MHz, DMSO-d₆) δ ppm: 7.48 (d, 1H), 7.25 (d, 1H), 7.22 (t, 1H), 7.20 (td, 1H), 3.93-3.85 (m, 4H), 3.87/3.85 (d+d, 2H), 3.53/3.51 (dd+dd, 2H), 2.62/2.31 (dd+dd, 2H), 1.94-1.42 (m, 8H), 1.77 (m, 1H), 0.89 (d, 3H), 0.88 (s, 9H), 0.02 (s, 6H). HRMS calculated for C₂₅H₄₁NO₃Si: 431.2856; found 432.2926 (M+H).

### Example 38F 2′-[(2R)-3-hydroxy-2-methyl-propyl]spiro[cyclohexane-4,1′-isoindoline]-1-one

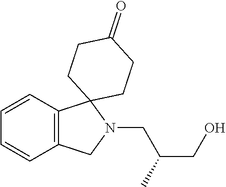 [SMILES: C[C@@H](CO)CN1Cc2ccccc2C12CCC(=O)CC2]

Using General procedure 9 and Example 38E as the appropriate ketal Example 38F was obtained. ¹H NMR (500 MHz, DMSO-d₆) δ ppm: 7.57 (d, 1H), 7.30 (d, 1H), 7.24/7.22 (t+t, 2H), 4.52 (t, 1H), 3.93/3.88 (d+d, 2H), 3.41/3.30 (m+m, 2H), 2.68/2.43 (m+m, 2H), 2.65/2.38 (m+m, 4H), 2.12/1.79 (m+m, 4H), 1.79 (m, 1H), 0.87 (d, 3H). HRMS calculated for C₁₇H₂₃NO₂: 273.1729; found 274.1796 (M+H).

### Example 38G 4-(3-chloroanilino)-2′-[(2R)-3-hydroxy-2-methylpropyl]-2′,3′-dihydrospiro[cyclohexane-1,1′-isoindole]-4-carbonitrile

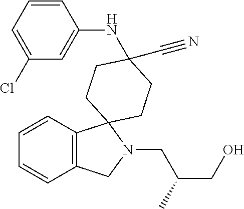 [SMILES: C[C@@H](CO)CN1Cc2ccccc2C12CCC(C#N)(Nc1cccc(Cl)c1)CC2]

Using General procedure 11 and Example 38F as the appropriate ketone and 3-chloroaniline as the appropriate aniline Example 38G was obtained as a mixture of diastereoisomers. ¹H NMR (500 MHz, DMSO-d₆) δ ppm: 7.58-7.18 (m, 4H), 7.21 (t, 1H), 6.92/6.90 (t, 1H), 6.87/6.86 (dm, 1H), 6.78/6.77 (dm, 1H), 6.50/6.45 (s, 1H), 4.56 (t, 1H), 3.93/3.91/3.89/3.87 (d+d, 2H), 3.74-3.27 (m, 2H), 2.68/2.56/2.44/2.34 (dd+dd, 2H), 2.52-1.41 (m, 8H), 1.81/1.77 (m, 1H), 0.89/0.86 (d, 3H). HRMS calculated for C₂₄H₂₈ClN₃O: 409.1921; found 410.1988 (M+H).

### Example 38H (1s,4S)-4-(3-chloroanilino)-2′-[(2R)-3-hydroxy-2-methylpropyl]-2′,3′-dihydrospiro[cyclohexane-1,1′-isoindole]-4-carboxamide

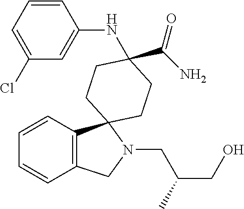 [SMILES: C[C@@H](CO)CN1Cc2ccccc2[C@]12CC[C@@](Nc1cccc(Cl)c1)(C(N)=O)CC2]

Using General procedure 12a and Example 38G as the appropriate nitrile a mixture of diastereoisomers was obtained. The diastereoisomers were separated via flash chromatography using DCM and MeOH as eluents. The diastereoisomer eluting earlier was collected as Example 38H. ¹H NMR (500 MHz, DMSO-d₆) δ ppm: 7.54 (m, 1H), 7.35/7.16 (s+s, 2H), 7.26 (m, 1H), 7.25-7.19 (m, 2H), 7.10 (t, 1H), 6.70 (t, 1H), 6.60 (dm, 1H), 6.59 (dm, 1H), 6.14 (s, 1H), 4.63 (br s, 1H), 3.92/3.83 (d+d, 2H), 3.45/3.28 (dd+dd, 2H), 2.67/2.44 (dd+dd, 2H), 2.28/2.23/1.98 (m+m, 4H), 1.96/1.89/1.30/1.25 (m+m, 4H), 1.79 (m, 1H), 0.86 (d, 3H). HRMS calculated for C₂₄H₃₀ClN₃O₂: 427.2027; found 428.2099 (M+H).

### Example 381 (1s,4S)-4-(3-chloroanilino)-2′-[(2R)-3-hydroxy-2-methylpropyl]-2′,3′-dihydrospiro[cyclohexane-1,1′-isoindole]-4-carboxylic acid

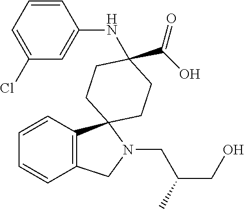 [SMILES: C[C@@H](CO)CN1Cc2ccccc2[C@]12CC[C@@](Nc1cccc(Cl)c1)(C(=O)O)CC2]

Using General procedure 13 and Example 38H as the appropriate amide Example 381 was obtained. ¹H NMR (500 MHz, DMSO-d₆) δ ppm: 7.60-7.26 (m, 4H), 7.08 (t, 1H), 6.69 (t, 1H), 6.59 (dm, 2H), 4.35-3.95 (br, 2H), 3.44/3.38 (dd+dd, 2H), 2.90/2.67 (br s+br s, 2H), 2.36-1.42 (br m, 8H), 1.93 (m, 1H), 0.94 (d, 3H). HRMS calculated for C₂₄H₂₉ClN₂O₃: 428.1867; found 429.1935 (M+H).

### Example 38J methyl (1s,4S)-4-(3-chloroanilino)-2′-[(2R)-3-hydroxy-2-methylpropyl]-2′,3′-dihydrospiro[cyclohexane-1,1′-isoindole]-4-carboxylate

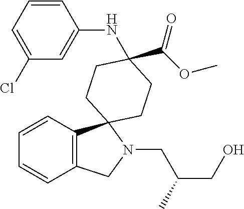 [SMILES: COC(=O)[C@]1(Nc2cccc(Cl)c2)CC[C@@]2(CC1)c1ccccc1CN2C[C@@H](C)CO]

Using General procedure 17a and Example 381 as the appropriate amino acid Example 38J was obtained. ¹H NMR (500 MHz, DMSO-d₆) δ ppm: 7.52/7.28/7.23 (m+m+m, 4H), 7.08 (t, 1H), 6.60 (m, 1H), 6.58 (m, 1H), 6.44 (dd, 1H), 6.36 (s, 1H), 4.59 (br, 1H), 3.91/3.86 (d+d, 2H), 3.65 (s, 3H), 3.43/3.27 (m+m, 2H), 2.62/2.41 (m+m, 2H), 2.28-2.10 (m, 4H), 1.97/1.32 (m+m, 4H), 1.79 (m, 1H), 0.86 (d, 3H). HRMS calculated for C₂₅H₃₁ClN₂O₃: 442.2023; found 443.2093 (M+H).

### Example 38 (1s,4S)-4-(3-chloroanilino)-2′-{(2R)-2-methyl-3-[(thieno[3,2-b]pyridin-7-yl)oxy]propyl}-2′,3′-dihydrospiro[cyclohexane-1,1′-isoindole]-4-carboxylic acid

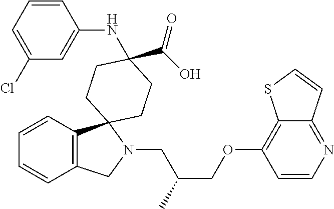 [SMILES: C[C@@H](COc1ccnc2ccsc12)CN1Cc2ccccc2[C@]12CC[C@@](Nc1cccc(Cl)c1)(C(=O)O)CC2]

Using General procedure 32 and Example 38J as the appropriate isoindoline and thieno[3,2-b]pyridin-7-ol as the appropriate alcohol Example 38 was obtained. ¹H NMR (500 MHz, DMSO-d₆) δ: 9.19 (br s, 1H), 8.46 (d, 1H), 7.83 (d, 1H), 7.58-7.17 (m, 4H), 7.46 (d, 1H), 7.02 (t, 1H), 6.93 (d, 1H), 6.61 (t, 1H), 6.54 (dm, 1H), 6.51 (dm, 1H), 6.04 (br s, 1H), 4.28/4.16 (dd+dd, 2H), 4/3.88 (d+d, 2H), 2.82/2.59 (dd+dd, 2H), 2.30-1.13 (m, 8H), 2.22 (m, 1H), 1.09 (d, 3H). HRMS calculated for C₃₁H₃₂ClN₃O₃S: 561.1853; found 562.1905 (M+H).

### Example 39 (1s,4S)-4-(3-chloroanilino)-2′-{(2R)-3-[(1H-indol-4-yl)oxy]-2-methylpropyl}-2′,3′-dihydrospiro[cyclohexane-1,1′-isoindole]-4-carboxylic acid

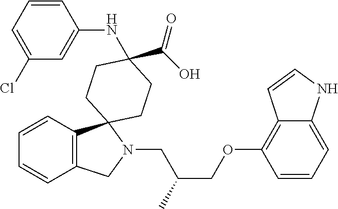 [SMILES: C[C@@H](COc1cccc2[nH]ccc12)CN1Cc2ccccc2[C@]12CC[C@@](Nc1cccc(Cl)c1)(C(=O)O)CC2]

Using General procedure 32 and Example 38J as the appropriate isoindoline and 1H-indol-4-ol as the appropriate alcohol Example 39 was obtained. ¹H NMR (500 MHz, DMSO-d₆) δ: 12.82 (br s, 1H), 11.00 (s, 1H), 7.53-7.22 (m, 4H), 7.09 (t, 1H), 7.05 (t, 1H), 6.94 (t, 1H), 6.92 (t, 1H), 6.61 (t, 1H), 6.56 (dd, 1H), 6.55 (dd, 1H), 6.43 (dd, 1H), 6.38 (t, 1H), 6.23 (br s, 1H), 4.11/3.90 (dd+dd, 2H), 3.97/3.92 (m+m, 2H), 2.80/2.63 (dd+dd, 2H), 2.30-1.20 (m, 8H), 2.19 (m, 1H), 1.09 (d, 3H). HRMS calculated for C₃₂H₃₄ClN₃O₃: 543.2289; found 544.2354 (M+H).

### Example 40

### Example 40A (1s,4S)-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxamide

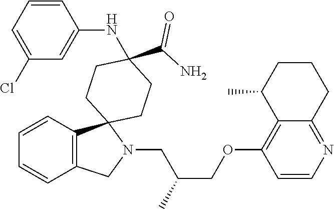 [SMILES: C[C@@H](COc1ccnc2c1[C@H](C)CCC2)CN1Cc2ccccc2[C@]12CC[C@@](Nc1cccc(Cl)c1)(C(N)=O)CC2]

Using General procedure 30a and Example 38H as the appropriate isoindoline and Preparation 2a1 as the appropriate alcohol Example 40A was obtained. ¹H NMR (500 MHz, DMSO-d₆) δ ppm: 8.11 (d, 1H), 7.56-7.18 (m, 4H), 7.32/7.14 (s+s, 2H), 7.09 (t, 1H), 6.73 (d, 1H), 6.68 (t, 1H), 6.6 (dm, 1H), 6.56 (dm, 1H), 6.08 (s, 1H), 4.02/3.94 (dd+dd, 2H), 3.95/3.88 (d+d, 2H), 3.06 (m, 1H), 2.79/2.59 (dd+dd, 2H), 2.74/2.63 (m+m, 2H), 2.16 (m, 1H), 2.06-1.14 (m, 12H), 1.13 (d, 3H), 1.08 (d, 3H). HRMS calculated for C₃₄H₄₁ClN₄O₂: 572.2918; found 573.3002 (M+H).

### Example 40 (1s,4S)-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

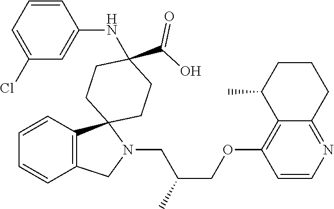 [SMILES: C[C@@H](COc1ccnc2c1[C@H](C)CCC2)CN1Cc2ccccc2[C@]12CC[C@@](Nc1cccc(Cl)c1)(C(=O)O)CC2]

Using General procedure 13 and Example 40A as the appropriate amide Example 40 was obtained. ¹H NMR (500 MHz, DMSO-d₆) δ: 12.79 (br, 1H), 8.1 (d, 1H), 7.52-7.22 (m, 4H), 7.04 (t, 1H), 6.73 (d, 1H), 6.6 (t, 1H), 6.53 (br d, 1H), 6.51 (dd, 1H), 6.2 (br, 1H), 4/3.95 (m+m, 2H), 3.95/3.89 (d+d, 2H), 3.03 (m, 1H), 2.77/2.57 (m+m, 2H), 2.72/2.62 (m+m, 2H), 2.25-1.21 (m, 8H), 2.16 (m, 1H), 1.76/1.65 (m+m, 2H), 1.53/1.46 (m+m, 2H), 1.11 (d, 3H), 1.08 (d, 3H). HRMS calculated for C₃₄H₄₀N₃O₃Cl: 573.2758; found: 574.2826 (M+H).

### Example 41

### Example 41A 6-fluoro-2,3-dihydro-1H-isoindol-1-one

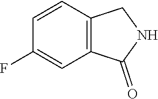 [SMILES: O=C1NCc2ccc(F)cc21]

To a solution of 7 M NH₃ in MeOH (250 mL) was added methyl 2-(bromomethyl)-5-fluorobenzoate (10.0 g, 40.5 mmol) and the mixture was stirred at rt for 18 h. The mixture was concentrated in vacuo and the residue was triturated with water, the solids were collected by filtration, washed with water and dried under vacuum to afford Example 41A as a solid (5.44 g, 36 mmol, 89%). LRMS calculated for C₈H₆FNO: 151; found: 152 (M+H). ¹H NMR (400 MHz, DMSO-d₆) δ ppm: 8.74 (br s, 1H), 7.65-7.60 (m, 1H), 7.50-7.40 (m, 2H), 4.36 (app s, 2H).

### Example 41B 2-[(2R)-3-{[tert-butyl(diphenyl)silyl]oxy}-2-methylpropyl]-6-fluoro-2,3-dihydro-1H-isoindol-1-one

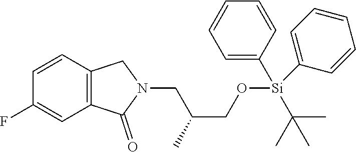 [SMILES: C[C@@H](CO[Si](c1ccccc1)(c1ccccc1)C(C)(C)C)CN1Cc2ccc(F)cc2C1=O]

To a solution of Example 41A (11.3 g, 75 mmol, 1 eq) in MeCN (400 mL) was added Preparation 3a (35.2 g, 90 mmol, 1.2 eq), 18-crown-6 ether (1.69 mL, 7.5 mmol, 0.1 eq) and Cs₂CO₃ (61.1 g, 188 mmol, 2.5 eq). The mixture was sparged with N₂ (5 min) and heated at reflux for 18 h. The mixture was concentrated in vacuo and the residue was partitioned between EtOAc and water. The organic phase was washed with brine, dried (MgSO₄) and concentrated in vacuo. Purification by automated flash chromatography (CombiFlash Rf, 330 g RediSep™ silica cartridge) eluting with a gradient of 0-18% EtOAc in heptane afforded Example 41B as a beige solid (18.3 g, 39.6 mmol, 53%). LRMS calculated for C₂₈H₃₂FNO₂Si: 461; found 462 (M+H). ¹H NMR (400 MHz, DMSO-d₆) δ ppm: 7.64-7.57 (m, 5H), 7.48-7.35 (m, 8H), 4.41 (d, J=17.6 Hz, 1H), 4.34 (d, J=17.6 Hz, 1H), 3.63-3.48 (m, 3H), 3.42 (dd, J=13.6, 7.4 Hz, 1H), 2.18-2.07 (m, 1H), 0.99 (s, 9H), 0.92 (d, J=6.7 Hz, 3H).

### Example 41C 2″-[(2R)-3-{[tert-butyl(diphenyl)silyl]oxy}-2-methylpropyl]-5″-fluorodispiro[[1,3]dioxolane-2,1′-cyclohexane-4′,1″-isoindol]-3″(2″H)-one

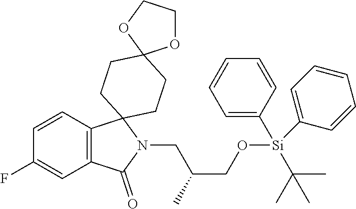 [SMILES: C[C@@H](CO[Si](c1ccccc1)(c1ccccc1)C(C)(C)C)CN1C(=O)c2cc(F)ccc2C12CCC1(CC2)OCCO1]

A solution of Example 41B (40.9 g, 88.5 mmol, 1 eq) in THE (950 mL) was sparged with N₂ (10 min) and cooled to −78° C. LiHMDS (195 mL, 1 M, 195 mmol, 2.2 eq) was added, the mixture was stirred for 30 min before the dropwise addition of Preparation 1b (30.6 g, 106 mmol, 1.2 eq) in THE (250 mL) and the mixture was stirred at rt for 15 h. The mixture was concentrated in vacuo and the residue was partitioned between EtOAc and sat. aq. NH₄Cl solution. The organic phase was washed with brine, dried (MgSO₄) and concentrated in vacuo. Purification by automated flash chromatography (CombiFlash Rf, 330 g RediSep™ silica cartridge) eluting with a gradient of 0-20% EtOAc in heptane afforded Example 41C as a yellow oil (37.5 g, 63.9 mmol, 72%). LRMS calculated for C₃₅H₄₂FNO₄Si: 587; found: 588 (M+H). ¹H NMR (400 MHz, DMSO-d₆) δ ppm: 7.90 (dd, J=8.5, 4.4 Hz, 1H), 7.66-7.54 (m, 4H), 7.49-7.33 (m, 8H), 4.00-3.88 (m, 4H), 3.58 (d, J=5.1 Hz, 2H), 3.39 (dd, J=13.9, 6.9 Hz, 1H), 3.25 (dd, J=13.9, 8.0 Hz, 1H), 2.41-2.19 (m, 3H), 2.13-2.01 (m, 2H), 1.88-1.76 (m, 2H), 1.33-1.22 (m, 2H), 1.04 (s, 9H), 0.89 (d, J=6.7 Hz, 3H).

### Example 41D 2″-[(2R)-3-{[tert-butyl(diphenyl)silyl]oxy}-2-methylpropyl]-5″-fluoro-2″,3″-dihydrodispiro[[1,3]dioxolane-2,1′-cyclohexane-4′,1″-isoindole]

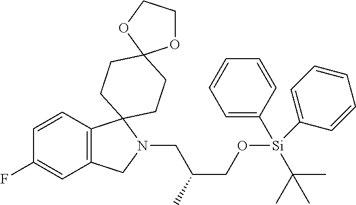 [SMILES: C[C@@H](CO[Si](c1ccccc1)(c1ccccc1)C(C)(C)C)CN1Cc2cc(F)ccc2C12CCC1(CC2)OCCO1]

To a solution of Example 41C (37.5 g, 63.9 mmol, 1 eq) in THF (600 mL), cooled to 0° C. was added LAH (95.8 mL, 1 M, 95.8 mmol, 1.5 eq) and the mixture was stirred at 0° C. for 1 h then warmed to rt for 2 h. The mixture was cooled to 0° C., water (3.65 mL) was added followed by 15% aq. NaOH solution (3.65 mL) then water (11 mL) and the mixture was stirred for 30 min. EtOAc and MgSO₄ were added to the mixture, stirred for 10 min, filtered through celite and concentrated in vacuo. Purification by automated flash chromatography (CombiFlash Rf, 330 g RediSep™ silica cartridge) eluting with a gradient of 0-10% EtOAc in heptane afforded Example 41D as a yellow oil (17.4 g, 30.3 mmol, 47%). LRMS calculated for C₃₅H₄₄FNO₃Si: 573; found: 574 (M+H). ¹H NMR (400 MHz, DMSO-d₆) δ ppm: 7.65-7.58 (m, 4H), 7.52-7.36 (m, 7H), 7.09 (dd, J=8.9, 2.6 Hz, 1H), 7.00 (td, J=8.8, 2.6 Hz, 1H), 3.95-3.84 (m, 4H), 3.77 (s, 2H), 3.61 (d, J=4.7 Hz, 2H), 2.66 (dd, J=11.8, 7.5 Hz, 1H), 2.37 (dd, J=12.0, 6.8 Hz, 1H), 1.97-1.78 (m, 5H), 1.74-1.65 (m, 2H), 1.47-1.33 (m, 2H), 1.03 (s, 9H), 0.95 (d, J=6.7 Hz, 3H).

### Example 41E 5′-fluoro-2′-[(2R)-3-hydroxy-2-methylpropyl]-2′,3′-dihydrospiro[cyclohexane-1,1′-isoindol]-4-one

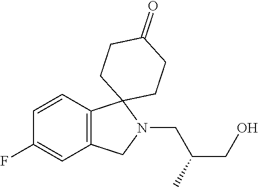 [SMILES: C[C@@H](CO)CN1Cc2cc(F)ccc2C12CCC(=O)CC2]

To a solution of Example 41D (17.4 g, 30.3 mmol, 1 eq) in acetone (105 mL) was added 2 M aq. HCl solution (106 mL, 2 M, 212 mmol, 7 eq) and the mixture was heated at 65° C. for 16 h. The mixture was allowed to cool to rt and partitioned between EtOAc and sat. aq. NaHCO₃ solution. The organic phase was washed with brine, dried (MgSO₄) and concentrated in vacuo. Purification by automated flash chromatography (CombiFlash Rf, 220 g RediSep™ silica cartridge) eluting with a gradient of 0-60% EtOAc in heptane afforded Example 41E as a yellow oil (7.11 g, 24.4 mmol, 81%). LRMS calculated for C₁₇H₂₂FNO₂: 291; found: 292 (M+H). ¹H NMR (400 MHz, DMSO-d₆) δ ppm: 7.61 (dd, J=8.4, 5.0 Hz, 1H), 7.16 (dd, J=8.9, 2.6 Hz, 1H), 7.03 (ddd, J=9.5, 8.4, 2.6 Hz, 1H), 4.51 (t, J=5.2 Hz, 1H), 3.95-3.86 (m, 2H), 3.44-3.37 (m, 1H), 3.33-3.26 (m, 1H), 2.72-2.57 (m, 3H), 2.46-2.32 (m, 3H), 2.17-2.06 (m, 2H), 1.84-1.71 (m, 3H), 0.86 (d, J=6.9 Hz, 3H).

### Example 41F (1'S,1″s)-5″-fluoro-2″-[(2R)-3-hydroxy-2-methylpropyl]-2″,3″-dihydrodispiro[imidazolidine-4,1′-cyclohexane-4′,1″-isoindole]-2,5-dione

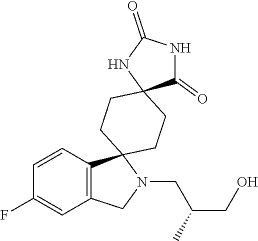 [SMILES: C[C@@H](CO)CN1Cc2cc(F)ccc2[C@]12CC[C@@]1(CC2)NC(=O)NC1=O]

To a solution of Example 41E (7.11 g, 24.4 mmol, 1 eq) in a mixture of EtOH (98 mL) and water (98 mL) was added NaCN (2.39 g, 48.8 mmol, 2 eq) and (NH₄)₂CO₃ (9.38 g, 97.6 mmol, 4 eq) and then heated at 60° C. for 16 h. A mixture of diastereoisomers was obtained. The mixture was cooled to rt, then cooled to 0° C. and a precipitate formed. The solids were collected by filtration, washed with water and dried under vacuum to afford a single diastereoisomer Example 41F as a white solid (4.33 g, 12 mmol, 49%). LRMS calculated for C₁₉H₂₄FN₃O₃: 361; found: 362 (M+H). ¹H NMR (400 MHz, DMSO-d₆) δ ppm: 10.75 (s, 1H), 8.91 (s, 1H), 7.50 (dd, J=8.5, 5.0 Hz, 1H), 7.19-7.07 (m, 2H), 4.58 (dd, J=5.8, 4.6 Hz, 1H), 3.95 (d, J=13.6 Hz, 1H), 3.88 (d, J=13.6 Hz, 1H), 3.49-3.41 (m, 1H), 3.29-3.21 (m, 1H), 2.83-2.76 (m, 1H), 2.57-2.52 (m, 1H), 2.22-2.07 (m, 2H), 1.94-1.81 (m, 2H), 1.81-1.71 (m, 1H), 1.69-1.59 (m, 2H), 1.51-1.41 (m, 2H), 0.88 (d, J=6.7 Hz, 3H).

### Example 41G (1s,4S)-4-amino-5′-fluoro-2′-[(2R)-3-hydroxy-2-methylpropyl]-2′,3′-dihydrospiro[cyclohexane-1,1′-isoindole]-4-carboxylic acid

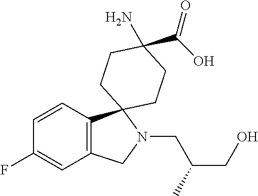 [SMILES: C[C@@H](CO)CN1Cc2cc(F)ccc2[C@]12CC[C@@](N)(C(=O)O)CC2]

To a solution of Example 41F (4.33 g, 12 mmol, 1 eq) in water (46 mL) was added LiOH×H₂O (5.03 g, 120 mmol, 10 eq) and the mixture was heated in a bomb calorimeter at 155° C. for 18 h. The mixture was cooled to 0° C., neutralised with 2 M aq. HCl solution (60 mL) and the solids were collected by filtration and washed with water and dried under vacuum to afford Example 41G as a white powder (3.32 g, 9.87 mmol, 82%). LRMS calculated for C₁₈H₂₅FN₂O₃: 336; found: 337 (M+H). ¹H NMR (400 MHz, DMSO-d₆) δ ppm: 7.76 (dd, J=8.5, 5.1 Hz, 1H), 7.71 (br s, 3H), 7.13 (dd, J=8.9, 2.6 Hz, 1H), 7.10-7.02 (m, 1H), 4.59 (br s, 1H), 3.94 (d, J=13.5 Hz, 1H), 3.87 (d, J=13.5 Hz, 1H), 3.48-3.41 (m, 1H), 3.29-3.21 (m, 1H), 2.77 (dd, J=12.1, 7.4 Hz, 1H), 2.56-2.49 (m, 1H), 2.49-2.37 (m, 2H), 1.86-1.71 (m, 3H), 1.71-1.62 (m, 2H), 1.39-1.31 (m, 2H), 0.87 (d, J=6.7 Hz, 3H).

### Example 41H (1s,4S)-4-(3-chloroanilino)-5′-fluoro-2′-[(2R)-3-hydroxy-2-methylpropyl]-2′,3′-dihydrospiro[cyclohexane-1,1′-isoindole]-4-carboxylic acid

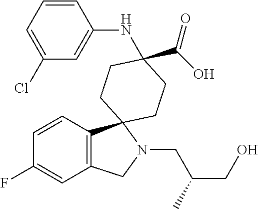 [SMILES: C[C@@H](CO)CN1Cc2cc(F)ccc2[C@]12CC[C@@](Nc1cccc(Cl)c1)(C(=O)O)CC2]

To a solution of Example 41G (3.32 g, 9.87 mmol, 1 eq) in DMF (90 mL) was added Cs₂CO₃ (6.43 g, 19.7 mmol, 2 eq), followed by 1-chloro-3-iodobenzene (1.34 mL, 10.9 mmol, 1.1 eq) and ethyl 2-cyclohexanonecarboxylate (632 μL, 3.95 mmol, 0.4 eq). The mixture was sparged with N₂ (10 min) before the addition of CuI (188 mg, 0.99 mmol, 0.1 eq) and the mixture was heated at 140° C. for 1 h in a sealed flask. The mixture was cooled to rt, filtered, the solids washed with DCM and the filtrate was concentrated in vacuo. The residue was dissolved in DCM, then loaded onto a MeOH-wet PE-AX cartridge (5×20 g), washed successively with DCM and MeOH, eluted with 15% HCOOH in DCM and concentrated in vacuo to afford Example 41H as a brown gum (4.47 g, 10 mmol, quant.). LRMS calculated for C₂₄H₂₈ClFN₂O₃: 446; found: 447 (M+H).

### Example 411 methyl (1s,4S)-4-(3-chloroanilino)-5′-fluoro-2′-[(2R)-3-hydroxy-2-methylpropyl]-2′,3′-dihydrospiro[cyclohexane-1,1′-isoindole]-4-carboxylate

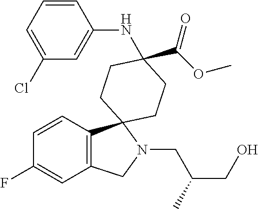 [SMILES: COC(=O)[C@]1(Nc2cccc(Cl)c2)CC[C@@]2(CC1)c1ccc(F)cc1CN2C[C@@H](C)CO]

To a solution of Example 41H (4.47 g, 10 mmol, 1 eq) in DCM (75 mL) and MeOH (75 mL), cooled to 0° C., was added TMS-CHNN (33.3 mL, 0.6 M, 20 mmol, 2 eq) dropwise. The mixture was stirred for 1 h, then further TMS-CHNN (33.3 mL, 0.6 M, 20 mmol, 2 eq) was added and stirred for 1 h at 0° C. and at rt for 1.5 h. The mixture was quenched with the slow addition of sat. aq. NaHCO₃ solution, partitioned between DCM and water, the aq. phase was extracted with DCM, and the combined organic extracts were washed with brine, dried (MgSO₄) and concentrated in vacuo. Purification by automated flash chromatography (CombiFlash Rf, 120 g RediSep™ silica cartridge) eluting with a gradient of 0-54% EtOAc in heptane afforded Example 411 as an off-white foam (2.08 g, 4.51 mmol, 45%). LRMS calculated for C₂₅H₃₀ClFN₂O₃: 460; found: 461 (M+H). ¹H NMR (400 MHz, DMSO-d₆) δ ppm: 7.52 (dd, J=8.5, 5.0 Hz, 1H), 7.15 (dd, J=8.9, 2.6 Hz, 1H), 7.12-7.03 (m, 2H), 6.62-6.57 (m, 2H), 6.47-6.42 (m, 1H), 6.38 (s, 1H), 4.58 (dd, J=5.8, 4.4 Hz, 1H), 3.93-3.83 (m, 2H), 3.65 (s, 3H), 3.47-3.38 (m, 1H), 3.31-3.22 (m, 1H), 2.61 (dd, J=11.9, 7.1 Hz, 1H), 2.40 (dd, J=11.9, 7.2 Hz, 1H), 2.25-2.11 (m, 4H), 2.03-1.90 (m, 2H), 1.82-1.71 (m, 1H), 1.36-1.26 (m, 2H), 0.86 (d, J=6.7 Hz, 3H).

### Example 41J methyl (1s,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

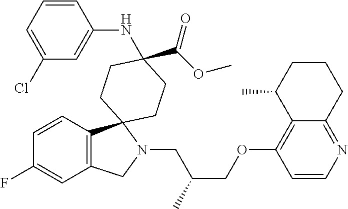 [SMILES: COC(=O)[C@]1(Nc2cccc(Cl)c2)CC[C@@]2(CC1)c1ccc(F)cc1CN2C[C@@H](C)COc1ccnc2c1[C@H](C)CCC2]

To a solution of Example 411 (2.08 g, 4.51 mmol, 1 eq) in THE (50 mL) was added Preparation 2a1 (1.1 g, 6.77 mmol, 1.5 eq) and PPh₃ (2.37 g, 9.02 mmol, 2 eq), followed by DTBAD (2.08 g, 9.02 mmol, 2 eq) and the mixture was stirred at 50° C. for 15 h. The mixture was partitioned between EtOAc and sat. aq. NaHCO₃ solution, and the organic phase was washed with brine, dried (MgSO₄) and concentrated in vacuo. Purification by automated flash chromatography (CombiFlash Rf, 120 g RediSep™ silica cartridge) eluting with a gradient of 0-9% MeOH in DCM afforded a solid. The solid was dissolved in MeOH, then loaded onto a MeOH-wet SCX cartridge (70 g), washed successively with DCM and MeOH, eluted with 7 M NH₃ in MeOH/DCM (1:5), and concentrated in vacuo to afford Example 41J as an off white foam (1.33 g, 2.19 mmol, 49%). LRMS calculated for C₃₅H₄₁ClFN₃O₃: 605; found: 606 (M+H). ¹H NMR (400 MHz, DMSO-d₆) δ ppm: 8.12 (d, J=5.6 Hz, 1H), 7.51 (dd, J=8.5, 5.0 Hz, 1H), 7.15 (dd, J=8.8, 2.6 Hz, 1H), 7.11-7.03 (m, 2H), 6.74 (d, J=5.7 Hz, 1H), 6.61-6.57 (m, 2H), 6.44-6.40 (m, 1H), 6.38 (s, 1H), 4.03-3.86 (m, 4H), 3.64 (s, 3H), 3.07-2.97 (m, 1H), 2.81-2.68 (m, 2H), 2.66-2.52 (m, 2H), 2.29-2.01 (m, 6H), 1.89 (td, J=13.5, 4.5 Hz, 1H), 1.83-1.70 (m, 1H), 1.71-1.60 (m, 1H), 1.55-1.41 (m, 2H), 1.39-1.31 (m, 1H), 1.28-1.19 (m, 1H), 1.11 (d, J=6.8 Hz, 3H), 1.08 (d, J=6.8 Hz, 3H).

### Example 41 (1s,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

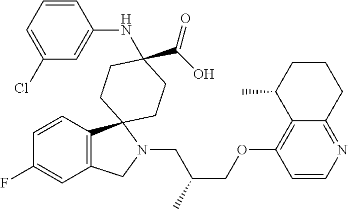 [SMILES: C[C@@H](COc1ccnc2c1[C@H](C)CCC2)CN1Cc2cc(F)ccc2[C@]12CC[C@@](Nc1cccc(Cl)c1)(C(=O)O)CC2]

To a solution of Example 41J (1.33 g, 2.19 mmol, 1 eq) in 1,4-dioxane (25 mL) and water (2.5 mL) was added LiOH×H₂O (368 mg, 8.78 mmol, 4 eq) and the mixture was heated at 110° C. for 16 h. The reaction was allowed to cool to rt and purification by reverse phase automated flash chromatography at pH 4 (CombiFlash Rf, C18 150 g Gold RediSep column) eluting with a gradient of 10-82% MeCN in water afforded Example 41 as a white solid (1.1 g, 1.86 mmol, 85%). HRMS calculated for C₃₄H₃₉N₃O₃FCl: 591.2664; found: 592.2720 (M+H). ¹H NMR (400 MHz, DMSO-d₆) δ ppm: 12.80 (br s, 1H), 8.11 (d, J=5.6 Hz, 1H), 7.51 (dd, J=8.5, 5.0 Hz, 1H), 7.15 (dd, J=8.9, 2.6 Hz, 1H), 7.11-7.04 (m, 2H), 6.74 (d, J=5.6 Hz, 1H), 6.60 (t, J=2.0 Hz, 1H), 6.59-6.55 (m, 1H), 6.53-6.49 (m, 1H), 6.29 (br s, 1H), 4.03-3.87 (m, 4H), 3.07-2.97 (m, 1H), 2.81-2.69 (m, 2H), 2.66-2.53 (m, 2H), 2.27-1.99 (m, 6H), 1.94-1.83 (m, 1H), 1.83-1.71 (m, 1H), 1.71-1.61 (m, 1H), 1.53-1.43 (m, 2H), 1.38-1.30 (m, 1H), 1.26-1.17 (m, 1H), 1.11 (d, J=6.8 Hz, 3H), 1.08 (d, J=6.8 Hz, 3H).

### Example 42

### Example 42A (1s,4S)-4-(3-chloro-4-fluoroanilino)-5′-fluoro-2′-[(2R)-3-hydroxy-2-methylpropyl]-2′,3′-dihydrospiro[cyclohexane-1,1′-isoindole]-4-carboxylic acid

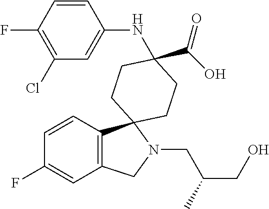 [SMILES: C[C@@H](CO)CN1Cc2cc(F)ccc2[C@]12CC[C@@](Nc1ccc(F)c(Cl)c1)(C(=O)O)CC2]

To a solution of Example 41G (1 g, 2.98 mmol, 1 eq) in DMF (18 mL) was added Cs₂CO₃ (1.94 g, 5.96 mmol, 2 eq), followed by 3-chloro-4-fluoroiodobenzene (416 μL, 3.28 mmol, 1.1 eq) and ethyl 2-cyclohexanonecarboxylate (191 μL, 1.19 mmol, 0.4 eq). The mixture was sparged with N₂ (10 min) before the addition of CuI (57 mg, 0.3 mmol, 0.1 eq) and the mixture was heated at 140° C. for 1 h under microwave irradiation. The mixture was cooled to rt, filtered through celite and the filtrate was concentrated in vacuo. The residue was dissolved in DCM, then loaded onto a DCM-wet PE-AX cartridge (20 g), washed successively with DCM and MeOH, eluted with 15% HCOOH in DCM, and concentrated in vacuo to afford Example 42A as a brown gum (456 mg, 0.98 mmol, 33%). LRMS calculated for C₂₄H₂₇ClF₂N₂O₃: 464; found: 465 (M+H).

### Example 42B methyl (1s,4S)-4-(3-chloro-4-fluoroanilino)-5′-fluoro-2′-[(2R)-3-hydroxy-2-methylpropyl]-2′,3′-dihydrospiro[cyclohexane-1,1′-isoindole]-4-carboxylate

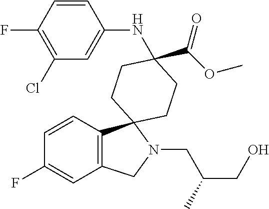 [SMILES: COC(=O)[C@]1(Nc2ccc(F)c(Cl)c2)CC[C@@]2(CC1)c1ccc(F)cc1CN2C[C@@H](C)CO]

To a solution of Example 42A (456 mg, 0.98 mmol, 1 eq) in DCM (7.5 mL) and MeOH (7.5 mL), cooled to 0° C., was added TMS-CHNN (4.9 mL, 0.6 M, 2.94 mmol, 3 eq) dropwise. The mixture was stirred for 1 h, then further TMS-CHNN (1.5 mL, 0.6 M) was added and stirred for 1.5 h at 0° C. The mixture was quenched with the slow addition of sat. aq. NaHCO₃ solution, partitioned between DCM and water, the aq. phase was extracted with DCM, and the combined organic extracts were washed with brine, dried (MgSO₄) and concentrated in vacuo. Purification by automated flash chromatography (CombiFlash Rf, 12 g Gold RediSep™ silica cartridge) eluting with a gradient of 0-70% EtOAc in heptane afforded Example 42B as a yellow gum (224 mg, 0.47 mmol, 48%). LRMS calculated for C₂₅H₂₉ClF₂N₂O₃: 478; found: 479 (M+H). ¹H NMR (400 MHz, DMSO-d₆) δ ppm: 7.52 (dd, J=8.4, 5.0 Hz, 1H), 7.18-7.02 (m, 3H), 6.68 (dd, J=6.3, 2.9 Hz, 1H), 6.46 (ddd, J=9.0, 3.8, 2.9 Hz, 1H), 6.24 (s, 1H), 4.55 (dd, J=5.7, 4.5 Hz, 1H), 3.93-3.83 (m, 2H), 3.65 (s, 3H), 3.47-3.37 (m, 1H), 3.31-3.23 (m, 1H), 2.61 (dd, J=11.9, 7.1 Hz, 1H), 2.40 (dd, J=11.9, 7.2 Hz, 1H), 2.25-2.09 (m, 4H), 2.00-1.87 (m, 2H), 1.82-1.72 (m, 1H), 1.36-1.27 (m, 2H), 0.86 (d, J=6.6 Hz, 3H).

### Example 42C methyl (1s,4S)-4-(3-chloro-4-fluoroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

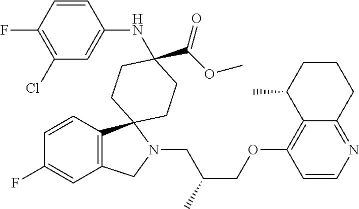 [SMILES: COC(=O)[C@]1(Nc2ccc(F)c(Cl)c2)CC[C@@]2(CC1)c1ccc(F)cc1CN2C[C@@H](C)COc1ccnc2c1[C@H](C)CCC2]

To a solution of Example 42B (224 mg, 0.47 mmol, 1 eq) in THE (6 mL) was added Preparation 2a1 (153 mg, 0.94 mmol, 2 eq) and PPh₃ (245 mg, 0.94 mmol, 2 eq), followed by DTBAD (215 mg, 0.94 mmol, 2 eq) and the mixture was stirred at 50° C. for 2.5 h. The mixture was partitioned between EtOAc and sat. aq. NaHCO₃ solution, and the organic phase was washed with brine, dried (MgSO₄) and concentrated in vacuo. Purification by automated flash chromatography (CombiFlash Rf, 24 g RediSep™ silica cartridge) eluting with a gradient of 0-100% EtOAc in heptane afforded Example 42C as a white solid (172 mg, 0.28 mmol, 59%). LRMS calculated for C₃₅H₄₀ClF₂N₃O₃: 623; found: 624 (M+H). ¹H NMR (400 MHz, DMSO-d₆) δ ppm: 8.11 (d, J=5.6 Hz, 1H), 7.50 (dd, J=8.5, 5.0 Hz, 1H), 7.17-7.09 (m, 2H), 7.09-7.03 (m, 1H), 6.74 (d, J=5.7 Hz, 1H), 6.68 (dd, J=6.3, 2.8 Hz, 1H), 6.43 (dt, J=9.0, 3.5 Hz, 1H), 6.25 (s, 1H), 4.02-3.93 (m, 3H), 3.89 (d, J=13.5 Hz, 1H), 3.64 (s, 3H), 3.05-2.94 (m, 1H), 2.81-2.69 (m, 2H), 2.67-2.53 (m, 2H), 2.29-1.99 (m, 6H), 1.93-1.71 (m, 2H), 1.70-1.58 (m, 1H), 1.54-1.40 (m, 2H), 1.39-1.32 (m, 1H), 1.26-1.18 (m, 1H), 1.11 (d, J=6.8 Hz, 3H), 1.08 (d, J=6.7 Hz, 3H).

### Example 42 (1s,4S)-4-(3-chloro-4-fluoroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

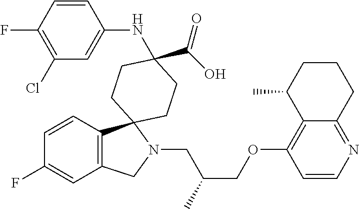 [SMILES: C[C@@H](COc1ccnc2c1[C@H](C)CCC2)CN1Cc2cc(F)ccc2[C@]12CC[C@@](Nc1ccc(F)c(Cl)c1)(C(=O)O)CC2]

To a solution of Example 42C (172 mg, 0.28 mmol, 1 eq) in 1,4-dioxane (4 mL) and water (0.4 mL) was added LiOH×H₂O (58 mg, 1.38 mmol, 5 eq) and the mixture was heated at 110° C. for 3 h. The reaction was allowed to cool to rt and purification by reverse phase automated flash chromatography at pH 4 (CombiFlash Rf, C18 50 g Gold RediSep column) eluting with a gradient of 10-52% MeCN in water afforded Example 42 as a white solid (123 mg, 0.2 mmol, 73%). HRMS calculated for C₃₄H₃₈N₃O₃F₂Cl: 609.2570; found: 610.2660 (M+H). ¹H NMR (400 MHz, DMSO-d₆) δ ppm: 8.11 (d, J=5.6 Hz, 1H), 7.50 (dd, J=8.5, 5.0 Hz, 1H), 7.17-7.02 (m, 3H), 6.74 (d, J=5.7 Hz, 1H), 6.67 (dd, J=6.2, 2.6 Hz, 1H), 6.54-6.48 (m, 1H), 6.13 (br s, 1H), 4.02-3.92 (m, 3H), 3.88 (d, J=13.4 Hz, 1H), 3.04-2.95 (m, 1H), 2.82-2.68 (m, 2H), 2.66-2.52 (m, 2H), 2.28-1.97 (m, 6H), 1.91-1.69 (m, 2H), 1.69-1.54 (m, 1H), 1.53-1.39 (m, 2H), 1.37-1.29 (m, 1H), 1.27-1.16 (m, 1H), 1.10 (d, J=6.9 Hz, 3H), 1.08 (d, J=6.8 Hz, 3H).

### Example 43

### Example 43A 4-bromo-2-fluoro-5-methylphenol

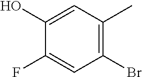

To a solution of 2-fluoro-5-methylphenol (50 g, 396.4 mmol, 1 eq) in AcOH (125 mL) was added Br₂ (20.31 mL, 396.4 mmol, 1 eq) dropwise at rt. The mixture was stirred at rt for 90 min and concentrated in vacuo. The residue was partitioned between EtOAc and water. The organic phase was washed with water, brine, dried (MgSO₄) and concentrated in vacuo to afford Example 43A as a pale, yellow oil (96 g, 374.6 mmol, 95%). ¹H NMR (400 MHz, CDCl₃) δ ppm: 7.27 (d, 1H), 6.92 (d, 1H), 2.34 (s, 3H).

### Example 43B 1-bromo-5-fluoro-4-methoxy-2-methylbenzene

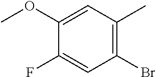

To a suspension of Example 43A (97 g, 378.5 mmol, 1 eq) and Cs₂CO₃ (123.3 g, 378.5 mmol, 1 eq) in acetone (750 mL) was added MeI (35.34 mL, 567.7 mmol, 1.5 eq). The mixture was heated at 50° C. for 4 h and the resulting suspension allowed to cool to rt. Water was added and extracted twice with EtOAc. The combined organic phase was washed with water, brine, dried (MgSO₄) and concentrated in vacuo. Purification by vacuum distillation afforded Example 43B as a colourless oil (76.6 g, 349.7 mmol, 92%). ¹H NMR (400 MHz, CDCl₃) δ ppm: 7.27 (d, 1H), 6.85 (d, 1H), 3.89 (s, 3H) 2.34 (s, 3H).

### Example 43C 5-fluoro-4-methoxy-2-methylbenzoic acid

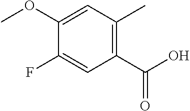

To a solution of Example 43B (70 g, 320 mmol, 1 eq) in THE (600 mL) at −78° C. under a N₂ atmosphere was added nBuLi, 2.5 M in hexanes, (200 mL, 480 mmol, 1.5 eq) dropwise. The resulting yellow solution was allowed to warm slowly to −25° C. to give a brown solution before being cooled again to −78° C. Solid CO₂ was added and the suspension was stirred for 1 h at −78° C. and allowed to warm to rt before concentrating in vacuo. Water was added, and the solution was washed with Et₂O twice and the Et₂O extracts were discarded. The aq. solution was acidified with AcOH to give a yellow suspension. The solids were removed by filtration, washed with water, dried in vacuo at 60° C. to afford Example 43C as a yellow solid (27.1 g, 147.2 mmol, 46%). ¹H NMR (400 MHz, CDCl₃) δ ppm: 7.84 (d, 1H), 6.82 (d, 1H), 3.97 (s, 3H) 2.66 (s, 3H).

### Example 43D methyl 5-fluoro-4-methoxy-2-methylbenzoate

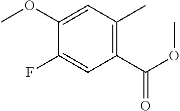

To a suspension of Example 43C (27.1 g, 132.4 mmol, 1 eq) in MeOH (400 mL) at 0° C. was added cc. H₂SO₄ (12 mL, 220.7 mmol, 1.5 eq) dropwise. The mixture was heated at 75° C. for 18 h and then cooled to rt. 10% aq. K₂CO₃ solution (800 mL) was added and the mixture was extracted with Et₂O twice. The combined organic phase was washed with water, brine, dried (MgSO₄) and concentrated in vacuo, which afforded Example 43D as a dark yellow oil that solidified on standing (23.4 g, 118 mmol, 89%). ¹H NMR (400 MHz, CDCl₃) δ ppm: 7.72 (d, 1H), 6.80 (d, 1H), 3.95 (s, 3H) 3.88 (s, 3H), 2.66 (s, 3H).

### Example 43E 6-fluoro-5-methoxy-2,3-dihydro-1H-isoindol-1-one

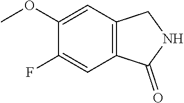

To a suspension of Example 43D (23.4 g, 118 mmol, 1 eq) and NBS (21.0 g, 118 mmol, 1 eq) in CCl₄ (300 mL) was added azobisbutyronitrile (194 mg, 1.18 mmol, 0.01 eq). The mixture was heated at 95° C. for 7 h and then cooled to rt. The solids were removed by filtration, washed with CCl₄ and the combined filtrate concentrated in vacuo. Then 7 M NH₃ solution in MeOH (400 mL, 2.79 mol, 25 eq) was added and the suspension was stirred at 0° C. for 15 min then at rt for 5 h. The mixture was concentrated in vacuo and water was added. The solids were removed by filtration and triturated in EtOAc. The solids were collected by filtration and dried under vacuum to afford Example 43E as an off white solid (12.2 g, 67.34 mmol, 60%). ¹H NMR (400 MHz, DMSO-d₆) δ ppm: 8.52 (s, br, 1H), 7.44 (d, 1H), 7.39 (d, 1H), 4.32 (q, 2H), 3.91 (s, 3H).

### Example 43F 6-fluoro-5-methoxy-2-{(2R)-3-[(4-methoxyphenyl)methoxy]-2-methylpropyl}-2,3-dihydro-1H-isoindol-1-one

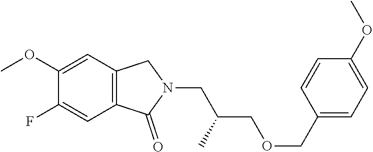 [SMILES: COc1ccc(COC[C@H](C)CN2Cc3cc(OC)c(F)cc3C2=O)cc1]

To a suspension of Example 43E (7.5 g, 41.4 mmol, 1 eq), and Cs₂CO₃ (13.49 g, 41.4 mmol, 1 eq) in MeCN (250 mL) was added Preparation 3c (16.96 g, 62.1 mmol, 1.5 eq). 18-Crown-6 ether (547 mg, 2.07 mmol, 0.05 eq) was added and the suspension was heated at 95° C. for 36 h. The mixture was allowed to cool to rt and EtOAc was added. The solids were removed by filtration and washed with EtOAc. The combined filtrate was washed with water, brine, dried (MgSO₄) and concentrated in vacuo. Purification by flash chromatography (100 g silica cartridge) eluting with 40% EtOAc in heptane afforded Example 43F as a brown gum (5.80 g, 15.53 mmol, 38%). ¹H NMR (400 MHz, CDCl₃) δ ppm: 7.53 (d, 1H), 7.24-7.19 (m, 2H), 6.96 (d, 1H), 6.89-6.83 (m, 2H), 4.42 (s, 2H), 4.26 (s, 2H), 3.96 (s, 3H), 3.82 (s, 3H), 3.60 (dd, 1H), 3.52 (dd, 1H), 3.43-3.34 (m, 2H), 2.25 (dtd, 1H), 0.99 (d, 3H).

### Example 43G 5″-fluoro-6″-methoxy-2″-{(2R)-3-[(4-methoxyphenyl)methoxy]-2-methylpropyl}dispiro[[1,3]dioxolane-2,1′-cyclohexane-4′,1″-isoindol]-3″(2″H)-one

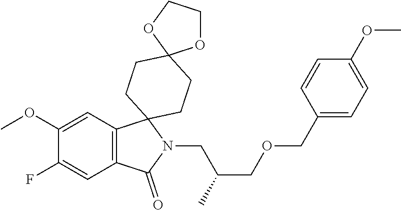

Using General procedure 8b and Example 43F as the appropriate isoindolin-1-one, Example 43G was obtained as an orange gum. ¹H NMR (400 MHz, CDCl₃) δ ppm: 7.55 (d, 1H), 7.29-7.26 (m, 3H), 6.87 (d, 2H), 4.43 (d, 2H), 4.07-4.01 (m, 4H), 3.99 (s, 3H), 3.82 (s, 3H), 3.50 (dd, 1H), 3.46-3.38 (m, 2H), 3.30 (dd, 1H), 2.62-2.52 (m, 1H), 2.40 (dtd, 2H), 2.21-2.11 (m, 2H), 1.94 (tt, 2H), 1.50 (dp, 2H), 1.02 (d, 3H).

### Example 43H 5″-fluoro-6″-methoxy-2″-{(2R)-3-[(4-methoxyphenyl)methoxy]-2-methylpropyl}-2″,3″-dihydrodispiro[[1,3]dioxolane-2,1′-cyclohexane-4′,1″-isoindole]

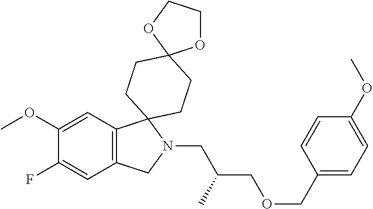 [SMILES: COc1ccc(COC[C@H](C)CN2Cc3cc(F)c(OC)cc3C23CCC2(CC3)OCCO2)cc1]

Using General procedure 38 and Example 43G as the appropriate isoindolin-1-one, Example 43H was obtained as an orange gum. H NMR (400 MHz, CDCl₃) δ ppm: 7.31-7.26 (m, 2H), 7.13 (d, 1H), 6.97 (d, 1H), 6.92-6.87 (m, 2H), 4.49-4.41 (m, 2H), 4.02 (s, 4H), 3.91 (s, 3H), 3.87 (d, 2H), 3.83 (s, 3H), 3.49 (dd, 1H), 3.34 (dd, 1H), 2.69 (dd, 1H), 2.43 (dd, 1H), 2.06-1.94 (m, 5H), 1.89-1.83 (m, 2H), 1.00 (d, 3H).

### Example 431 5′-fluoro-2′-[(2R)-3-hydroxy-2-methylpropyl]-6′-methoxy-2′,3′-dihydrospiro[cyclohexane-1,1′-isoindol]-4-one

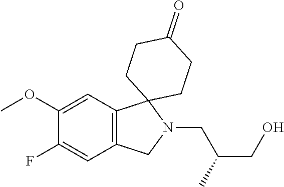 [SMILES: COc1cc2c(cc1F)CN(C[C@@H](C)CO)C21CCC(=O)CC1]

A solution of Example 43H (4.60 g, 9.47 mmol, 1 eq) in THE (100 mL) was added to a solution of PTSA (5.41 g, 28.42 mmol, 3 eq) in water (100 mL). The mixture was heated at 85° C. for 24 h and then cooled to rt. The mixture was neutralised by the addition sat. aq. NaHCO₃ solution and extracted with EtOAc twice. The combined organic phase was washed with water, brine, dried (MgSO₄) and concentrated in vacuo, which afforded Example 431 as a pale brown gum that solidified (2.80 g, 7.84 mmol, 83%). ¹H NMR (400 MHz, CDCl₃) δ ppm: 7.02 (d, 1H), 6.97 (d, 1H), 5.41 (bs, 1H), 4.39 (d, 1H), 3.91 (s, 3H), 3.77-3.69 (m, 2H), 3.57 (dd, 1H), 2.87-2.67 (m, 4H), 2.63-2.47 (m, 2H), 2.36 (ddd, 1H), 2.26-2.15 (m, 3H), 1.85 (dtd, 1H), 0.86 (d, 3H).

### Example 43J (1'S,1″s)-5″-fluoro-2″-[(2R)-3-hydroxy-2-methylpropyl]-6″-methoxy-2″,3″-dihydrodispiro[imidazolidine-4,1′-cyclohexane-4′,1″-isoindole]-2,5-dione

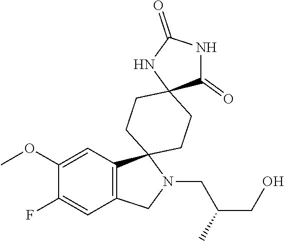 [SMILES: COc1cc2c(cc1F)CN(C[C@@H](C)CO)[C@]21CC[C@]2(CC1)NC(=O)NC2=O]

Using General procedure 14 and Example 431 as the appropriate ketone, a single diastereoisomer, Example 43J was obtained as a white solid (2.2 g, 5.62 mmol, 65%). LRMS calculated for C₂₀H₂₆FN₃O₄: 391; found 392 (M+H).

### Example 43K (1s,4S)-4-amino-5′-fluoro-2′-[(2R)-3-hydroxy-2-methylpropyl]-6′-methoxy-2′,3′-dihydrospiro[cyclohexane-1,1′-isoindole]-4-carboxylic acid

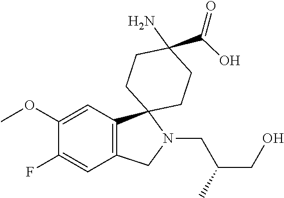 [SMILES: COc1cc2c(cc1F)CN(C[C@@H](C)CO)[C@]21CC[C@](N)(C(=O)O)CC1]

To a suspension of Example 43J (2.20 g, 5.62 mmol, 1 eq) in water (25 mL) was added LiOH×H₂O (2.12 g, 50.6 mmol, 9 eq). The mixture was heated in a PressureSyn reactor at 175° C. for 18 h and then cooled to rt. Water was added and the mixture was acidified to pH 6 using AcOH. The solution was concentrated in vacuo and EtOH (50 mL) was added. The solids were removed by filtration and the filtrate concentrated in vacuo. EtOH (25 mL) was added, the solids were removed by filtration and the filtrate concentrated in vacuo. The residue was triturated in EtOAc, the solids were collected by filtration and dried under vacuum at 60° C. to afford Example 43K as a pale brown solid (1.14 g, 3.1 mmol, 55%). LRMS calculated for C₁₉H₂₇FN₂O₄: 366; found 367 (M+H).

### Example 43L (1s,4S)-4-(3-chloroanilino)-5′-fluoro-2′-[(2R)-3-hydroxy-2-methylpropyl]-6′-methoxy-2′,3′-dihydrospiro[cyclohexane-1,1′-isoindole]-4-carboxylic acid

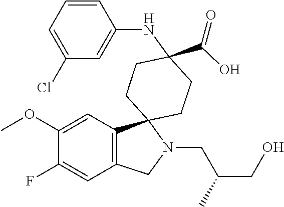 [SMILES: COc1cc2c(cc1F)CN(C[C@@H](C)CO)[C@]21CC[C@](Nc2cccc(Cl)c2)(C(=O)O)CC1]

To a suspension of Example 43K (2.2 g, 5.62 mmol, 1 eq) and Cs₂CO₃ (2 g, 6.14 mmol, 2 eq) in DMF (10 mL) was added 2-(2-methylpropanoyl)cyclohexan-1-one (0.1 mL, 0.61 mmol, 0.2 eq), 3-chloroiodobenzene (0.46 mL, 3.68 mmol, 1.2 eq) and finally, CuI (29 mg, 0.15 mmol, 0.05 eq). The mixture was heated at 110° C. for 18 h and then cooled to rt. Water was added, and the mixture was acidified to pH 5 with AcOH and extracted with EtOAc twice. The combined organic phase was washed with water, brine, dried (MgSO₄) and concentrated in vacuo. Purification by flash chromatography eluting with 10% MeOH in DCM afforded Example 43L as a dark brown gum (510 mg, 1.07 mmol, 35%). LRMS calculated for C₂₅H₃₀ClFN₂O₄: 476; found 477 (M+H).

### Example 43M methyl (1s,4S)-4-(3-chloroanilino)-5′-fluoro-2′-[(2R)-3-hydroxy-2-methylpropyl]-6′-methoxy-2′,3′-dihydrospiro[cyclohexane-1,1′-isoindole]-4-carboxylate

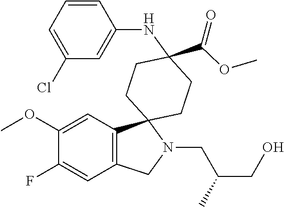 [SMILES: COc1cc2c(cc1F)CN(C[C@@H](C)CO)[C@]21CC[C@](Nc2cccc(Cl)c2)(C(=O)OC)CC1]

To a solution of Example 43L (510 mg, 1.07 mmol, 1 eq) in DCM (15 mL) and MeOH (15 mL) at 0° C., was added TMS-CHNN (5 mL, 0.6 M, 3 mmol, 2.81 eq) dropwise and the mixture was stirred for a further 0.5 h at 0° C. and 1 h at rt. The mixture was concentrated in vacuo and purification by flash chromatography eluting with 40% EtOAc in heptane afforded Example 43M as a pale orange solid (150 mg, 0.31 mmol, 29%). LRMS calculated for C₂₆H₃₂ClFN₂O₄: 490; found 491 (M+H).

### Example 43N methyl (1s,4S)-4-(3-chloroanilino)-5′-fluoro-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

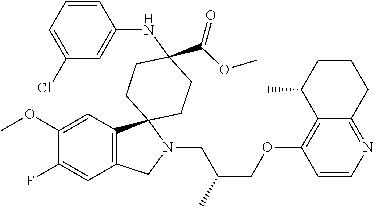 [SMILES: COc1cc2c(cc1F)CN(C[C@@H](C)COc1ccnc3c1[C@H](C)CCC3)[C@]21CC[C@](Nc2cccc(Cl)c2)(C(=O)OC)CC1]

To a solution of Example 43M (145 mg, 0.3 mmol, 1 eq) in toluene (15 mL) was added Preparation 2a1 (96 mg, 0.59 mmol, 2 eq), PPh₃ (155 mg, 0.59 mmol, 2 eq) and finally DTBAD (136 mg, 0.59 mmol, 2 eq). The mixture was stirred at rt for 3 h and then concentrated in vacuo. The mixture was purified by flash chromatography eluting with 50% EtOAc in heptane. Further purification was performed on an SCX cartridge pre-wetted with MeOH, washed successively with DCM, MeOH and eluted with 10% MeOH in DCM containing TEA (2.5%) and concentrated in vacuo. Finally, the solid obtained was triturated in heptane, the solids were collected by filtration and dried under vacuum to give Example 43N as a brown solid (75 mg, 0.12 mmol, 40%). LRMS calculated for C₃₆H₄₃ClFN₃O₄: 635; found 636 (M+H).

### Example 43 (1s,4S)-4-(3-chloroanilino)-5′-fluoro-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

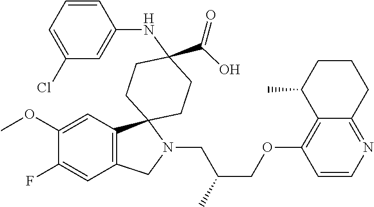 [SMILES: COc1cc2c(cc1F)CN(C[C@@H](C)COc1ccnc3c1[C@H](C)CCC3)[C@]21CC[C@](Nc2cccc(Cl)c2)(C(=O)O)CC1]

To a solution of Example 43N (70 mg, 0.11 mmol, 1 eq) in a mixture of MeOH (0.5 mL) and water (1.5 mL) was added LiOH×H₂O (18 mg, 0.44 mmol, 4 eq) and the mixture was stirred at 95° C. for 12 h. The mixture was cooled to rt and acidified to pH 5 using AcOH. The solids were collected by filtration, washed with water and dried under vacuum. Purification by flash chromatography eluting with 10% MeOH in DCM afforded Example 43 as a pale brown powder (62 mg, 0.1 mmol, 91%). LRMS calculated for C₃₅H₄₁ClFN₃O₄: 621; found 622 (M+H). ¹H NMR (400 MHz, DMSO-d₆) δ ppm: 8.11 (d, J=5.6 Hz, 1H), 7.32 (d, J=7.5 Hz, 1H), 7.15 (d, J=11.0 Hz, 1H), 7.02 (t, J=8.1 Hz, 1H), 6.73 (d, J=5.7 Hz, 1H), 6.60 (t, J=2.1 Hz, 1H), 6.56-6.47 (m, 2H), 6.13 (br s, 1H), 4.04-3.85 (m, 3H), 3.82 (s, 3H), 3.82 (d, J=12.4 Hz, 1H), 3.07-2.99 (m, 1H), 2.81-2.68 (m, 2H), 2.66-2.53 (m, 2H), 2.39-2.17 (m, 2H), 2.17-1.92 (m, 4H), 1.89-1.70 (m, 2H), 1.70-1.60 (m, 1H), 1.56-1.43 (m, 2H), 1.38-1.30 (m, 1H), 1.27-1.17 (m, 1H), 1.12 (d, J=6.9 Hz, 3H), 1.08 (d, J=6.8 Hz, 3H).

### Example 44 and Example 45

### Example 44A 6-bromo-N-(1,4-dioxaspiro[4.5]dec-7-en-8-yl)-N-{(2R)-3-[(4-methoxyphenyl)methoxy]-2-methylpropyl}-2H-1,3-benzodioxole-5-carboxamide

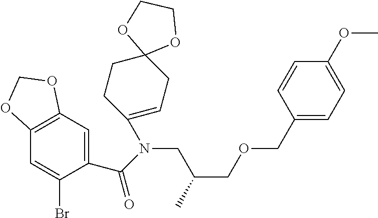 [SMILES: COc1ccc(COC[C@H](C)CN(C(=O)c2cc3c(cc2Br)OCO3)C2=CCC3(CC2)OCCO3)cc1]

Preparation 3e (1 eq.) was dissolved in dry toluene (10 mL/mmol amine) in a flask equipped with a Dean-Stark apparatus. 1,4-dioxaspiro[4.5]decan-8-one (1 eq.) was added to the mixture and stirred at reflux temperature until no further conversion was observed. The mixture was concentrated under reduced pressure and the residue was taken up in dry DCM (5 mL/mmol amine). TEA (1.9 eq.) and a solution of 6-bromo-1,3-benzodioxole-5-carbonyl chloride (1.1 eq.) in dry DCM (1 mL/mmol amine) were added to the mixture dropwise and stirred at rt until no further conversion was observed. The reaction mixture was diluted with water, sat. aq. NH₄Cl solution and extracted with DCM. The combined organic layers were dried over Na₂SO₄, filtered and the filtrate was concentrated under reduced pressure. The crude product was purified via flash chromatography using heptane and EtOAc as eluents to obtain Example 44A. ¹H NMR (500 MHz, DMSO-d₆) δ ppm: 7.24 (dm, 2H), 7.17 (s, 1H), 6.90 (dm, 2H), 6.89 (s, 1H), 6.07 (s, 2H), 5.40 (t, 1H), 4.41/4.35 (d+d, 2H), 3.79 (m, 4H), 3.74 (s, 3H), 3.62-3.31 (br, 2H), 3.39/3.26 (dd+dd, 2H), 2.20/1.97/1.51 (br+br+br, 6H), 1.98 (m, 1H), 0.93 (d, 3H). HRMS calculated for C₂₈H₃₂BrNO₇: 573.1362; found 574.1432 (M+H).

### Example 44B 6″-{(2R)-3-[(4-methoxyphenyl)methoxy]-2-methylpropyl}-2″H-dispiro[[1,3]dioxolane-2,1′-cyclohex[2]ene-4′,5″-[1,3]dioxolo[4,5-f]isoindol]-7″(6″H)-one

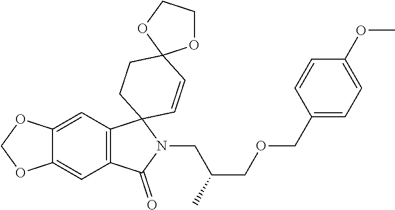 [SMILES: COc1ccc(COC[C@H](C)CN2C(=O)c3cc4c(cc3C23C=CC2(CC3)OCCO2)OCO4)cc1]

Using General procedure 39 and Example 44A as the appropriate benzamide Example 44B was obtained as a mixture of diastereoisomers. ¹H NMR (500 MHz, DMSO-d₆) δ ppm: 7.23/7.22 (dm/dm, 2H), 7.11/7.10 (s/s, 1H), 6.88/6.86 (dm/dm, 2H), 6.87 (s, 1H), 6.13 (m, 2H), 5.96/5.94 (d/d, 1H), 5.36/5.30 (d/d, 1H), 4.41-4.30 (m, 2H), 4.04-3.85 (m, 4H), 3.73 (s, 3H), 3.38-3.08 (m, 2H), 3.35-3.23 (m, 2H), 2.29 (m, 1H), 2.27/1.80 (m+m, 2H), 2.14/1.97 (tm+dm, 2H), 0.88/0.84 (d/d, 3H). HRMS calculated for C₂₈H₃₁NO₇: 493.2101; found 494.2168 (M+H).

### Example 44C 6″-{(2R)-3-[(4-methoxyphenyl)methoxy]-2-methylpropyl}-2″H-dispiro[[1,3]dioxolane-2,1′-cyclohexane-4′,5″-[1,3]dioxolo[4,5-f]isoindol]-7″(6″H)-one

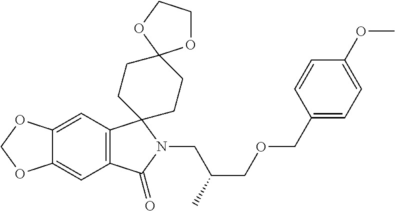 [SMILES: COc1ccc(COC[C@H](C)CN2C(=O)c3cc4c(cc3C23CCC2(CC3)OCCO2)OCO4)cc1]

Example 44B (1 eq.) was dissolved in EtOH (10-20 mL/mmol). 10% Pd/C (0.5 g catalyst/g isoindoline-1-one) and HCOONH₄ (20 eq.) were added and the mixture was stirred under N₂ atmosphere at 40-80° C. until no further conversion was observed. Then it was filtered through a pad of Celite, washed with EtOAc and the filtrate was concentrated under reduced pressure. The crude product was purified via flash chromatography using heptane and EtOAc as eluents to obtain Example 44C. ¹H NMR (500 MHz, DMSO-d₆) δ ppm: 7.31 (s, 1H), 7.24 (m, 2H), 7.11 (s, 1H), 6.89 (m, 2H), 6.14 (s, 2H), 4.36/4.34 (d+d, 2H), 4.01-3.87 (m, 4H), 3.73 (s, 3H), 3.31/3.29 (dd+dd, 2H), 3.28/3.13 (dd+dd, 2H), 2.44 (m, 1H), 2.32-1.25 (m, 8H), 0.88 (d, 3H). HRMS calculated for C₂₈H₃₃NO₇: 495.2257; found 496.232 (M+H).

### Example 44D 6″-{(2R)-3-[(4-methoxyphenyl)methoxy]-2-methylpropyl}-6″,7″-dihydro-2″H-dispiro[[1,3]dioxolane-2,1′-cyclohexane-4′,5″-[1,3]dioxolo[4,5-f]isoindole]

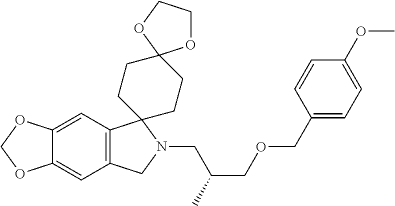 [SMILES: COc1ccc(COC[C@H](C)CN2Cc3cc4c(cc3C23CCC2(CC3)OCCO2)OCO4)cc1]

Using General procedure 38 and Example 44C as the appropriate ketal Example 44D was obtained. ¹H NMR (500 MHz, DMSO-d₆) δ ppm: 7.24 (d, 2H), 6.98 (s, 1H), 6.89 (d, 2H), 6.82 (s, 1H), 5.98 (s, 2H), 4.37/4.34 (d+d, 2H), 3.89 (m, 4H), 3.76/3.72 (d+d, 2H), 3.73 (s, 3H), 3.37/3.3 (dd+dd, 2H), 2.56/2.29 (dd+dd, 2H), 1.86-1.38 (m, 8H), 1.86 (m, 1H), 0.90 (d, 3H). HRMS calculated for C₂₈H₃₅NO₆: 481.2464; found 482.2525 (M+H).

### Example 44E (2R)-3-(2″H-dispiro[[1,3]dioxolane-2,1′-cyclohexane-4′,5″-[1,3]dioxolo[4,5-f]isoindol]-6″(7″H)-yl)-2-methylpropan-1-ol

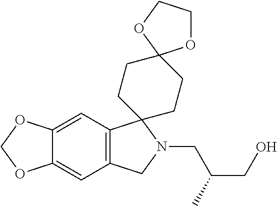 [SMILES: C[C@@H](CO)CN1Cc2cc3c(cc2C12CCC1(CC2)OCCO1)OCO3]

Using General procedure 28b and Example 44D as the appropriate PMB derivative Example 44E was obtained. ¹H NMR (500 MHz, DMSO-d₆) δ ppm: 6.98 (s, 1H), 6.84 (s, 1H), 5.97 (s, 2H), 4.58 (t, 1H), 3.89 (m, 4H), 3.79/3.74 (d+d, 2H), 3.40/3.28 (m+m, 2H), 2.54/2.32 (dd+dd, 2H), 1.91-1.40 (m, 8H), 1.73 (m, 1H), 0.84 (d, 3H). HRMS calculated for C₂₀H₂₇NO₅: 361.1889; found 362.1954 (M+H).

### Example 44F 6″-[(2R)-2-methyl-3-{[(5R)-5-methyl-5,6,7,8-tetrahydroquinolin-4-yl]oxy}propyl]-6″,7″-dihydro-2″H-dispiro[[1,3]dioxolane-2,1′-cyclohexane-4′,5″-[1,3]dioxolo[4,5-f]isoindole]

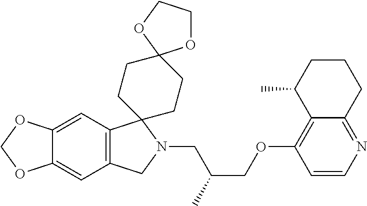 [SMILES: C[C@@H](COc1ccnc2c1[C@H](C)CCC2)CN1Cc2cc3c(cc2C12CCC1(CC2)OCCO1)OCO3]

Using General procedure 30a and Example 44E as the appropriate isoindoline and Preparation 2a1 as the appropriate alcohol Example 44F was obtained. ¹H NMR (500 MHz, DMSO-d₆) δ ppm: 8.12 (d, 1H), 6.96 (s, 1H), 6.83 (s, 1H), 6.74 (d, 1H), 5.96 (s, 2H), 3.98 (d, 2H), 3.94-3.83 (m, 4H), 3.87/3.73 (d+d, 2H), 3.17 (m, 1H), 2.77/2.67 (m+m, 2H), 2.75/2.45 (dd+dd, 2H), 2.09 (m, 1H), 1.94-1.25 (m, 12H), 1.18 (d, 3H), 1.07 (d, 3H). HRMS calculated for C₃₀H₃₈N₂O₅: 506.2781; found 507.2833 (M+H).

### Example 44G 6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindol]-4-one

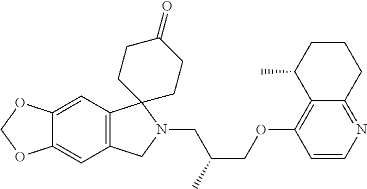 [SMILES: C[C@@H](COc1ccnc2c1[C@H](C)CCC2)CN1Cc2cc3c(cc2C12CCC(=O)CC2)OCO3]

Using General procedure 9 and Example 44F as the appropriate ketal Example 44G was obtained. ¹H NMR (500 MHz, DMSO-d₆) δ ppm: 8.13 (d, 1H), 7.16 (s, 1H), 6.84 (s, 1H), 6.74 (d, 1H), 5.97 (s, 2H), 4/3.97 (dd+dd, 2H), 3.9/3.78 (d+d, 2H), 3.1 (m, 1H), 2.81/2.54 (dd+dd, 2H), 2.77/2.66 (m+m, 2H), 2.63-1.62 (m, 8H), 2.13 (m, 1H), 1.83/1.75 (m+m, 2H), 1.69/1.64 (m+m, 2H), 1.15 (d, 3H), 1.07 (d, 3H). HRMS calculated for C₂₈H₃₄N₂O₄: 462.2519; found 463.2576 (M+H).

### Example 44H 6-[(2R)-2-methyl-3-{[(5R)-5-methyl-5,6,7,8-tetrahydroquinolin-4-yl]oxy}propyl]-6,7-dihydro-2H-dispiro[[1,3]dioxolo[4,5-f]isoindole-5,1′-cyclohexane-4′,4″-imidazolidine]-2″,5″-dione

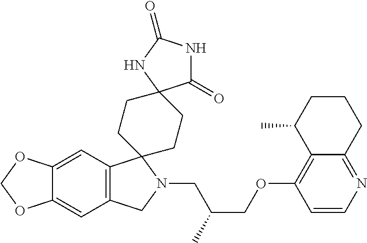 [SMILES: C[C@@H](COc1ccnc2c1[C@H](C)CCC2)CN1Cc2cc3c(cc2C12CCC1(CC2)NC(=O)NC1=O)OCO3]

Using General procedure 14 and Example 44G as the appropriate ketone a mixture of diastereoisomers was obtained. The crude intermediate was purified via flash chromatography using DCM and MeOH (1.2% NH₃) as eluents to give Example 44H as a mixture of diastereoisomers. ¹H NMR (500 MHz, DMSO-d₆) δ ppm: 10.72/10.55 (s/s, 1H), 8.86/8,18 (s/s, 1H), 8.12/8,11 (d/d, 1H), 7,10/6.96 (s/s, 1H), 6.86/6.81 (s, 1H), 6.68/6.73 (d/d, 1H), 6.02-5.93 (m, 2H), 4.07-3.70 (m, 4H), 3.31/3.22 (m, 1H), 3.32-2.54 (m, 4H), 2.21-1.25 (m, 13H), 1.18/1.15 (d/d, 3H), 1.09/1.08 (d/d, 3H).

### Example 441 4-amino-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

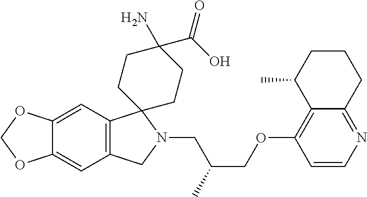 [SMILES: C[C@@H](COc1ccnc2c1[C@H](C)CCC2)CN1Cc2cc3c(cc2C12CCC(N)(C(=O)O)CC2)OCO3]

Using General procedure 15 and Example 44H as the appropriate hydantoin a mixture of diastereoisomers was obtained. The crude intermediate was purified via flash chromatography using DCM and MeOH (1.2% NH₃) as eluents to give Example 441 as a mixture of diastereoisomers. ¹H NMR (400 MHz, DMSO-d₆) δ ppm: 8.19/8.18 (d/d, 1H), 7.32/7.23 (s/s, 1H), 6.84 (m, 1H), 6.84 (m, 1H), 5.98/5.97 (s/s, 2H), 4.04/4.02/3.92 (m+m/br, 2H), 3.95-3.69 (m, 2H), 3.2 (m, 1H), 3-2.41 (m, 2H), 2.81/2.7 (dm+m, 2H), 2.51-1.23 (m, 12H), 2.14 (m, 1H), 1.17/1.15 (d/d, 3H), 1.08/1.07 (d/d, 3H). HRMS calculated for C₂₉H₃₇N₃O₅: 507.2733; found 508.2795 and 508.2798 (M+H).

### Example 44 (1s,4S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

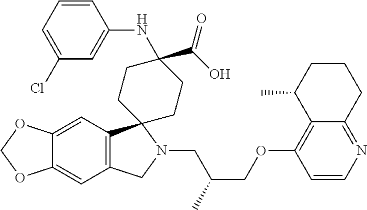 [SMILES: C[C@@H](COc1ccnc2c1[C@H](C)CCC2)CN1Cc2cc3c(cc2[C@]12CC[C@@](Nc1cccc(Cl)c1)(C(=O)O)CC2)OCO3]

and

### Example 45 (1r,4R)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

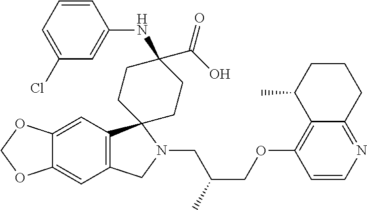 [SMILES: C[C@@H](COc1ccnc2c1[C@H](C)CCC2)CN1Cc2cc3c(cc2[C@]12CC[C@](Nc1cccc(Cl)c1)(C(=O)O)CC2)OCO3]

Using General procedure 16 and Example 441 as the appropriate amino acid and 1-chloro-3-iodo-benzene as the appropriate iodobenzene a mixture of diastereoisomers was obtained. The crude intermediate was purified via prep RP-HPLC using 25 mM aq. NH₄HCO₃ solution and MeCN as eluents. The diastereoisomer eluting earlier was collected as Example 44. HRMS calculated for C₃₅H₄₀ClN₃O₅: 617.2656; found 618.2708 (M+H).

The diastereoisomer eluting later was collected as Example 45. HRMS calculated for C₃₅H₄₀ClN₃O₅: 617.2656; found 618.2713 (M+H).

### Example 46

### Example 46A 5-chloro-4-methoxy-2-methylbenzoic acid

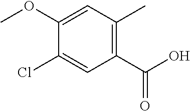

To an oven-dried flask was added 1-bromo-5-chloro-4-methoxy-2-methylbenzene (30 g, 127 mmol, 1 eq) in THE (300 mL). The solution was cooled to −78° C. and nBuLi (83.1 mL, 2.5 M, 191 mmol, 1.5 eq) was added dropwise. The solution was stirred at −78° C. for 1 h then solid CO₂ was added. The mixture was stirred at −78° C. for 1 h, then warmed to rt. The mixture was concentrated in vacuo and the residue was dissolved in water, acidified to pH 1 with 2 M aq. HCl solution and the resultant solids were collected by filtration, washed with water and dried under vacuum to afford Example 46A as a yellow solid (21 g, 105 mmol, 82%). LRMS calculated for C₉H₉ClO₃: 200; found: 199 (M−H). ¹H NMR (400 MHz, DMSO-d₆) δ ppm: 12.80 (br s, 1H), 7.85 (s, 1H), 7.08 (s, 1H), 3.91 (s, 3H), 2.55 (s, 3H).

### Example 46B methyl 5-chloro-4-methoxy-2-methylbenzoate

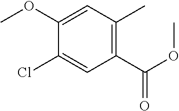

To a solution of Example 46A (26.8 g, 133.6 mmol, 1 eq) in MeOH (400 mL), cooled to 0° C., was added cc. H₂SO₄ (40 mL) and the mixture was heated at reflux for 16 h. The mixture was allowed to cool to rt, poured onto ice/water and stirred for 30 min. The resulting precipitate was collected by filtration and dried in vacuo to afford Example 46B as a beige solid (26.6 g, 123.9 mmol, 93%). ¹H NMR (400 MHz, DMSO-d₆) δ ppm: 7.86 (s, 1H), 7.13 (s, 1H), 3.92 (s, 3H), 3.80 (s, 3H), 2.55 (s, 3H).

### Example 46C methyl 2-(bromomethyl)-5-chloro-4-methoxybenzoate

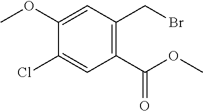

To a solution of Example 46B (8.25 g, 38.4 mmol, 1 eq) in CCl₄ (76 mL) was added NBS (7.18 g, 40.4 mmol, 1.05 eq), followed by benzoyl peroxide (0.4 mL, 1.92 mmol, 0.05 eq) and the mixture was heated at 80° C. for 24 h. The mixture was allowed to cool to rt, partitioned between DCM and water, and the organic phase was washed with brine, dried (MgSO₄) and concentrated in vacuo. Purification by automated flash chromatography (CombiFlash Rf, 220 g RediSep™ silica cartridge) eluting with a gradient of 0-25% EtOAc in heptane afforded Example 46C as a yellow solid (8.51 g, 29 mmol, 75%). ¹H NMR (400 MHz, DMSO-d₆) δ ppm: 7.93 (s, 1H), 7.44 (s, 1H), 5.04 (s, 2H), 3.95 (s, 3H), 3.85 (s, 3H).

### Example 46D 6-chloro-5-methoxy-2-{(2R)-3-[(4-methoxyphenyl)methoxy]-2-methylpropyl}1-2,3-dihydro-1H-isoindol-1l-one

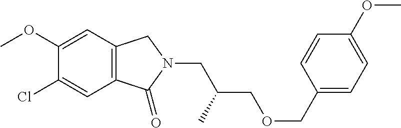

To a solution of Preparation 3e (6.9 g, 33 mmol, 1.5 eq) in MeCN (80 mL) was added DIPEA (7.66 mL, 44 mmol, 2 eq) and the mixture was heated at 80° C. for 30 min. A solution of Example 46C (6.45 g, 22 mmol, 1 eq) in MeCN (70 mL) was added dropwise and the mixture was heated at 80° C. for 12 h. The mixture was cooled to rt, concentrated in vacuo and the residue was partitioned between EtOAc and water. The organic phase was washed with brine, dried (MgSO₄) and concentrated in vacuo. Purification by automated flash chromatography (CombiFlash Rf, 120 g RediSep™ silica cartridge) eluting with a gradient of 0-75% EtOAc in heptane afforded Example 46D as a yellow oil (8.36 g, 20.2 mmol, 92%). LRMS calculated for C₂₁H₂₄ClNO₄: 389; found: 390 (M+H). ¹H NMR (400 MHz, CDCl₃) δ ppm: 7.82 (s, 1H), 7.22-7.17 (m, 2H), 6.91 (s, 1H), 6.86-6.82 (m, 2H), 4.44-4.37 (m, 2H), 4.31-4.21 (m, 2H), 3.97 (s, 3H), 3.81 (s, 3H), 3.60 (dd, J=13.9, 7.4 Hz, 1H), 3.51 (dd, J=13.9, 7.3 Hz, 1H), 3.43-3.33 (m, 2H), 2.31-2.18 (m, 1H), 0.98 (d, J=6.8 Hz, 3H).

### Example 46E 5″-chloro-6″-methoxy-2″-{(2R)-3-[(4-methoxyphenyl)methoxy]-2-methylpropyl}dispiro[[1,3]dioxolane-2,1′-cyclohexane-4′,1″-isoindol]-3″(2″H)-one

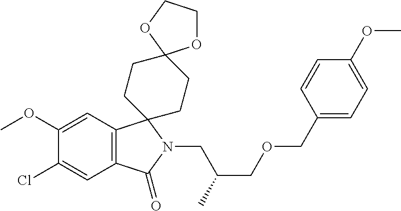

A solution of Example 46D (9.73 g, 25 mmol, 1 eq) in THF (125 mL) was sparged with N₂ (10 min) and cooled to −78° C. LiHMDS (56.2 mL, 1 M, 56.2 mmol, 2.25 eq) was added, the mixture was stirred for 1 h before the dropwise addition of Preparation 1b (8.98 g, 31.2 mmol, 1.25 eq) in THE (75 mL). The mixture was stirred at −78° C. for 1 h, then at rt for 72 h. The mixture was partitioned between EtOAc and sat. aq. NH₄Cl solution, and the organic phase was washed with brine, dried (MgSO₄) and concentrated in vacuo. Purification by automated flash chromatography (CombiFlash Rf, 120 g RediSep™ silica cartridge) eluting with a gradient of 0-100% EtOAc in heptane afforded Example 46E as a yellow gum (10.9 g, 18.9 mmol, 76%). LRMS calculated for C₂₈H₃₄ClNO₆: 515; found: 516 (M+H). ¹H NMR (400 MHz, CDCl₃) δ ppm: 7.86 (s, 1H), 7.30-7.25 (m, 2H), 7.23 (s, 1H), 6.90-6.85 (m, 2H), 4.48-4.40 (m, 2H), 4.10-3.98 (m, 7H), 3.82 (s, 3H), 3.50 (dd, J=14.1, 7.6 Hz, 1H), 3.46-3.38 (m, 2H), 3.29 (dd, J=14.1, 7.6 Hz, 1H), 2.63-2.50 (m, 1H), 2.48-2.32 (m, 2H), 2.21-2.10 (m, 2H), 1.99-1.88 (m, 2H), 1.54-1.47 (m, 2H), 1.02 (d, J=6.9 Hz, 3H).

### Example 46F 5″-chloro-6″-methoxy-2″-{(2R)-3-[(4-methoxyphenyl)methoxy]-2-methylpropyl}-2″,3″-dihydrodispiro[[1,3]dioxolane-2,1′-cyclohexane-4′,1″-isoindole]

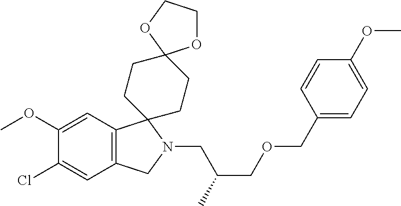

To a solution of Example 46E (9.76 g, 18.9 mmol, 1 eq) in THE (200 mL), cooled to 0° C. was added LAH (28.4 mL, 1 M in THF, 28.4 mmol, 1.5 eq) and the mixture was stirred at 0° C. for 2 h then warmed to rt for 18 h. The mixture was cooled to 0° C., water (1 mL) was added followed by 2 M aq. NaOH solution (2 mL) then water (5 mL) and the mixture was stirred for 30 min. EtOAc was added to the mixture, stirred for 10 min, filtered through celite, washed with EtOAc and the filtrate was concentrated in vacuo. Purification by automated flash chromatography (CombiFlash Rf, 120 g RediSep™ silica cartridge) eluting with a gradient of 0-40% EtOAc in heptane afforded Example 46F as an orange gum (7.91 g, 15.8 mmol, 83%). LRMS calculated for C₂₈H₃₆ClNO₅: 501; found: 502 (M+H). ¹H NMR (400 MHz, CDCl₃) δ ppm: 7.31-7.26 (m, 2H), 7.24 (s, 1H), 7.10 (s, 1H), 6.92-6.87 (m, 2H), 4.49-4.41 (m, 2H), 4.02 (app s, 4H), 3.92 (s, 3H), 3.90-3.83 (m, 2H), 3.83 (s, 3H), 3.49 (dd, J=9.0, 5.0 Hz, 1H), 3.34 (dd, J=9.0, 6.4 Hz, 1H), 2.69 (dd, J=12.0, 7.3 Hz, 1H), 2.42 (dd, J=12.0, 7.0 Hz, 1H), 2.07-1.83 (m, 7H), 1.72-1.58 (m, 2H), 1.01 (d, J=6.7 Hz, 3H).

### Example 46G 5′-chloro-2′-[(2R)-3-hydroxy-2-methylpropyl]-6′-methoxy-2′,3′-dihydrospiro[cyclohexane-1,1′-isoindol]-4-one

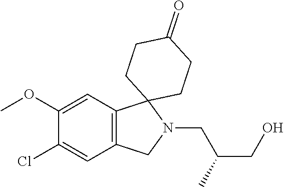 [SMILES: COc1cc2c(cc1Cl)CN(C[C@@H](C)CO)C21CCC(=O)CC1]

To a solution of Example 46F (6.4 g, 12.8 mmol, 1 eq) in THE (150 mL) and water (150 mL) was added PTSA (3.83 mL, 38.2 mmol, 3 eq) and the mixture was heated at 85° C. for 18 h. Further PTSA (3.83 mL, 38.2 mmol, 3 eq) was added and heated at 85° C. for 24 h. The mixture was allowed to cool to rt and partitioned between EtOAc and sat. aq. NaHCO₃ solution, and the combined organic extracts were washed with brine, dried (MgSO₄) and concentrated in vacuo. Purification by reverse phase automated flash chromatography (CombiFlash Rf, C18 150 g RediSep column) eluting with a gradient of 10-100% MeCN in water afforded Example 46G as a colourless oil (2.60 g, 7.71 mmol, 60%). LRMS calculated for C₁₈H₂₄ClNO₃: 337; found: 338 (M+H). ¹H NMR (400 MHz, CDCl₃) δ ppm: 7.30 (s, 1H), 6.92 (s, 1H), 5.32 (br s, 1H), 4.39 (d, J=13.0 Hz, 1H), 3.92 (s, 3H), 3.78-3.69 (m, 2H), 3.57 (dd, J=10.8, 9.6 Hz, 1H), 2.87-2.67 (m, 4H), 2.64-2.46 (m, 2H), 2.36 (ddd, J=14.3, 10.0, 6.9 Hz, 1H), 2.29-2.15 (m, 3H), 1.86 (dtd, J=13.4, 5.6, 1.8 Hz, 1H), 0.86 (d, J=6.9 Hz, 3H).

### Example 46H (1'S,1″s)-5″-chloro-2″-[(2R)-3-hydroxy-2-methylpropyl]-6″-methoxy-2″,3″-dihydrodispiro[imidazolidine-4,1′-cyclohexane-4′,1″-isoindole]-2,5-dione

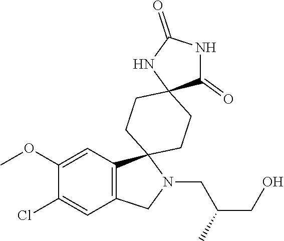 [SMILES: COc1cc2c(cc1Cl)CN(C[C@@H](C)CO)[C@]21CC[C@]2(CC1)NC(=O)NC2=O]

To a solution of Example 46G (2.58 g, 7.64 mmol, 1 eq) in a mixture of EtOH (60 mL) and water (60 mL) was added NaCN (749 mg, 15.3 mmol, 2 eq) and (NH₄)₂CO₃ (2.94 g, 30.6 mmol, 4 eq). The mixture was heated at 60° C. for 18 h. A mixture of diastereoisomers was obtained. The mixture was cooled to rt and a precipitate formed. The solids were collected by filtration, washed with water and dried under vacuum to afford a single diastereoisomer Example 46H as a white solid (776 mg, 1.9 mmol, 25%). LRMS calculated for C₂₀H₂₆ClN₃O₄: 407; found: 408 (M+H). ¹H NMR (400 MHz, DMSO-d₆) δ ppm: 10.74 (d, J=1.5 Hz, 1H), 8.90 (d, J=1.5 Hz, 1H), 7.37 (s, 1H), 7.24 (s, 1H), 4.57 (br t, J=4.8 Hz, 1H), 3.89 (d, J=12.8 Hz, 1H), 3.88 (s, 3H), 3.82 (d, J=12.8 Hz, 1H), 3.49-3.41 (m, 1H), 3.30-3.22 (m, 1H), 2.77 (dd, J=12.3, 7.4 Hz, 1H), 2.56-2.49 (m, 1H), 2.23 (qd, J=14.4, 4.3 Hz, 2H), 1.89 (tt, J=14.0, 5.1 Hz, 2H), 1.82-1.71 (m, 1H), 1.71-1.62 (m, 2H), 1.54-1.44 (m, 2H), 0.87 (d, J=6.6 Hz, 3H).

### Example 461 tert-butyl (1'S,1″s)-2″-{(2R)-3-[(tert-butoxycarbonyl)oxy]-2-methylpropyl}-5″-chloro-6″-methoxy-2,5-dioxo-2″,3″-dihydrodispiro[imidazolidine-4,1′-cyclohexane-4′,1″-isoindole]-1-carboxylate or tert-butyl (1'S,1″s)-2″-{(2R)-3-[(tert-butoxycarbonyl)oxy]-2-methylpropyl}-5″-chloro-6″-methoxy-2,5-dioxo-2″,3″-dihydrodispiro[imidazolidine-4,1′-cyclohexane-4′,1″-isoindole]-3-carboxylate (1/1)

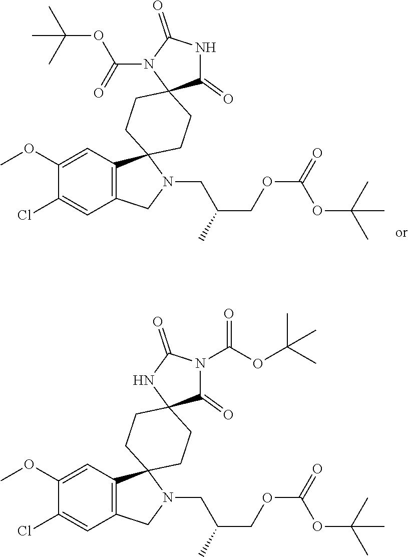

To a solution of Example 46H (206 mg, 0.51 mmol, 1 eq) in THE (10 mL) was added DMAP (2.5 mg, 0.02 mmol, 0.04 eq) and TEA (0.08 mL, 0.56 mmol, 1.1 eq). The mixture was stirred at rt for 5 min before the addition of Boc₂O (0.54 mL, 2.53 mmol, 5 eq) in THE (2 mL) and the mixture was stirred at rt for 16 h. The mixture was partitioned between EtOAc and water, and the organic phase was washed with brine, dried (MgSO₄) and concentrated in vacuo. Purification by automated flash chromatography (CombiFlash Rf, 24 g RediSep™ silica cartridge) eluting with a gradient of 0-100% EtOAc in heptane afforded one single regioisomer, Example 461 as a colourless oil (311 mg, 0.51 mmol, 39%). LRMS calculated for C₃₀H₄₂ClN₃O₈: 607; found: 608 (M+H).

### Example 46J (1s,4S)-4-amino-5′-chloro-2′-[(2R)-3-hydroxy-2-methylpropyl]-6′-methoxy-2′,3′-dihydrospiro[cyclohexane-1,1′-isoindole]-4-carboxylic acid

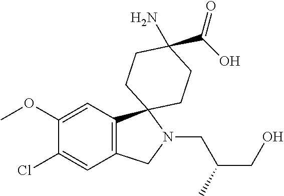 [SMILES: COc1cc2c(cc1Cl)CN(C[C@@H](C)CO)[C@]21CC[C@](N)(C(=O)O)CC1]

To a mixture of Example 461 (610 mg, 1.00 mmol, 1 eq) and water (20 mL) was added LiOH×H₂O (0.42 g, 10 mmol, 10 eq) and the mixture was heated in a sealed tube at 140° C. for 16 h. The mixture was allowed to cool to rt, filtered and the filtrate was neutralised with 2 M aq. HCl solution. The organics were extracted with EtOAc, and the combined organic extracts were washed with brine, dried (MgSO₄) and concentrated in vacuo. Purification by reverse phase automated flash chromatography (CombiFlash Rf, C18 50 g RediSep column) eluting with a gradient of 10-100% MeCN in water afforded Example 46J as a white solid (158 mg, 0.41 mmol, 41%). LRMS calculated for C₁₉H₂₇ClN₂O₄: 382; found: 383 (M+H). ¹H NMR (400 MHz, DMSO-d₆) δ ppm: 7.74 (s, 1H), 7.72 (br s, 3H), 7.32 (s, 1H), 4.59 (br s, 1H), 3.89 (s, 3H), 3.88 (d, J=12.8 Hz, 1H), 3.81 (d, J=12.8 Hz, 1H), 3.44 (dd, J=10.3, 5.7 Hz, 1H), 3.30-3.21 (m, 1H), 2.75 (dd, J=12.2, 7.4 Hz, 1H), 2.65-2.49 (m, 3H), 1.89-1.65 (m, 5H), 1.41-1.32 (m, 2H), 0.86 (d, J=6.6 Hz, 3H).

### Example 46K (1s,4S)-5′-chloro-4-(3-chloroanilino)-2′-[(2R)-3-hydroxy-2-methylpropyl]-6′-methoxy-2′,3′-dihydrospiro[cyclohexane-1,1′-isoindole]-4-carboxylic acid

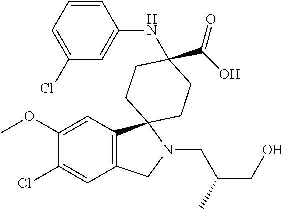 [SMILES: COc1cc2c(cc1Cl)CN(C[C@@H](C)CO)[C@]21CC[C@](Nc2cccc(Cl)c2)(C(=O)O)CC1]

To a solution of Example 46J (200 mg, 0.52 mmol, 1 eq) in DMF (6 mL) was added Cs₂CO₃ (340 mg, 1.04 mmol, 2 eq), followed by 3-chloroiodobenzene (0.08 mL, 0.63 mmol, 1.2 eq) and 2-isobutyrylcyclohexanone (18 mg, 0.1 mmol, 0.2 eq) in DMF (2 mL). The mixture was sparged with N₂ (10 min) before the addition of CuI (5 mg, 0.03 mmol, 0.05 eq) and the mixture was heated at 110° C. for 18 h. The mixture was cooled to rt, partitioned between EtOAc and water, and the organic phase was washed with brine, dried (MgSO₄) and concentrated in vacuo. Purification by automated flash chromatography (CombiFlash Rf, 4 g RediSep™ silica cartridge) eluting with a gradient of 0-10% MeOH in DCM afforded Example 46K as a beige solid (142 mg, 0.29 mmol, 55%). LRMS calculated for C₂₅H₃₀Cl₂N₂O₄: 492; found: 493 (M+H). ¹H NMR (400 MHz, DMSO-d₆) δ ppm: 7.41 (br s, 1H), 7.24 (s, 1H), 7.09 (t, J=8.0 Hz, 1H), 6.63-6.56 (m, 2H), 6.53 (dd, J=8.3, 2.2 Hz, 1H), 6.33 (br s, 1H), 4.01-3.80 (br m, 2H), 3.88 (s, 3H), 3.42 (dd, J=10.3, 5.9 Hz, 1H), 2.93-2.58 (br m, 1H), 2.43-2.21 (br m, 2H), 2.17 (d, J=13.6 Hz, 2H), 2.10-1.92 (br m, 2H), 1.90-1.73 (br m, 1H), 1.59-1.31 (br m, 2H), 0.89 (br d, J=6.4 Hz, 3H).

### Example 46L methyl (1s,4S)-5′-chloro-4-(3-chloroanilino)-2′-[(2R)-3-hydroxy-2-methylpropyl]-6′-methoxy-2′,3′-dihydrospiro[cyclohexane-1,1′-isoindole]-4-carboxylate

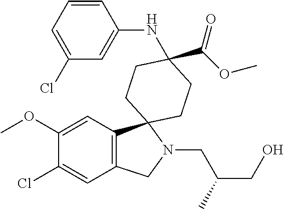 [SMILES: COc1cc2c(cc1Cl)CN(C[C@@H](C)CO)[C@]21CC[C@](Nc2cccc(Cl)c2)(C(=O)OC)CC1]

To a solution of Example 46K (714 mg, 1.45 mmol, 1 eq) in DCM (20 mL) and MeOH (20 mL), cooled to 0° C., was added TMS-CHNN (2.65 mL, 0.6 M, 1.59 mmol, 1.1 eq) dropwise. The mixture was stirred for 30 min at 0° C., then at rt for 1 h. Further TMS-CHNN (2.65 mL, 0.6 M, 1.59 mmol, 1.1 eq) was added and stirred at rt for 24 h. The mixture was concentrated in vacuo. Purification by automated flash chromatography (CombiFlash Rf, 24 g RediSep™ silica cartridge) eluting with a gradient of 0-10% MeOH in DCM afforded Example 46L as a beige foam (659 mg, 1.3 mmol, 90%). LRMS calculated for C₂₆H₃₂Cl₂N₂O₄: 506; found: 507 (M+H). ¹H NMR (400 MHz, DMSO-d₆) δ ppm: 7.36 (s, 1H), 7.21 (s, 1H), 7.09 (t, J=8.3 Hz, 1H), 6.63-6.55 (m, 2H), 6.44 (ddd, J=8.3, 2.2, 0.9 Hz, 1H), 6.37 (s, 1H), 4.56 (t, J=5.1 Hz, 1H), 3.89 (s, 3H), 3.87-3.78 (m, 2H), 3.67 (s, 3H), 3.46-3.38 (m, 1H), 3.31-3.23 (m, 1H), 2.60 (dd, J=11.9, 7.2 Hz, 1H), 2.39 (dd, J=11.9, 7.1 Hz, 1H), 2.36-2.24 (m, 2H), 2.21-2.12 (m, 2H), 2.01-1.89 (m, 2H), 1.81-1.71 (m, 1H), 1.40-1.32 (m, 2H), 0.86 (d, J=6.7 Hz, 3H).

### Example 46M methyl (1s,4S)-5′-chloro-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

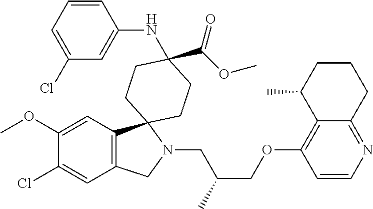 [SMILES: COc1cc2c(cc1Cl)CN(C[C@@H](C)COc1ccnc3c1[C@H](C)CCC3)[C@]21CC[C@](Nc2cccc(Cl)c2)(C(=O)OC)CC1]

To a solution of Example 46L (625 mg, 1.23 mmol, 1 eq) in toluene (25 mL) was added Preparation 2a1 (402 mg, 2.46 mmol, 2 eq) and PPh₃ (646 mg, 2.46 mmol, 2 eq), followed by DTBAD (567 mg, 2.46 mmol, 2 eq) and the mixture was stirred for 18 h at rt. The mixture was partitioned between EtOAc and water, and the organic phase was washed with brine, dried (MgSO₄) and concentrated in vacuo. Purification by automated flash chromatography (CombiFlash Rf, 24 g RediSep™ silica cartridge) eluting with a gradient of 0-100% EtOAc in heptane afforded a solid. The solid was dissolved in DCM, then loaded onto a DCM-wet SCX

cartridge (20 g), washed successively with DCM and MeOH, eluted with 10% 1.4 M NH₃/MeOH in DCM, and concentrated in vacuo to afford Example 46M as a beige foam (401 mg, 0.61 mmol, 50%). LRMS calculated for C₃₆H₄₃Cl₂N₃O₄: 651; found: 652 (M+H). ¹H NMR (400 MHz, CDCl₃) δ ppm: 8.16 (d, J=5.6 Hz, 1H), 7.21 (s, 1H), 7.17 (s, 1H), 7.04 (t, J=8.1 Hz, 1H), 6.68 (dd, J=7.9, 1.9 Hz, 1H), 6.59-6.52 (m, 2H), 6.41 (dd, J=7.9, 1.9 Hz, 1H), 4.02-3.94 (m, 3H), 3.91 (s, 3H), 3.81 (d, J=12.4 Hz, 1H), 3.69 (s, 3H), 3.17-3.08 (m, 1H), 2.94-2.66 (m, 3H), 2.53 (dd, J=12.1, 5.9 Hz, 1H), 2.49-2.27 (m, 2H), 2.23-2.11 (m, 3H), 2.04-1.69 (m, 4H), 1.63-1.55 (m, 2H), 1.53-1.39 (m, 2H), 1.19 (d, J=6.9 Hz, 3H), 1.13 (d, J=6.6 Hz, 3H).

### Example 46 (1s,4S)-5′-chloro-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

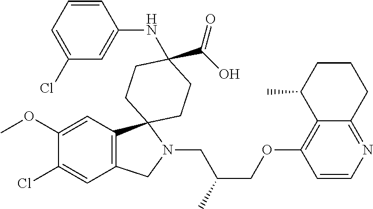 [SMILES: COc1cc2c(cc1Cl)CN(C[C@@H](C)COc1ccnc3c1[C@H](C)CCC3)[C@]21CC[C@](Nc2cccc(Cl)c2)(C(=O)O)CC1]

To a solution of Example 46M (200 mg, 0.31 mmol, 1 eq) in 1,4-dioxane (4.5 mL) and water (0.5 mL) was added LiOH×H₂O (64 mg, 1.53 mmol, 5 eq) and the mixture was heated at 110° C. for 1 h under microwave irradiation. The reaction was allowed to cool to rt, partitioned between EtOAc and water, and the aq. phase was acidified to pH 5 with 1 M aq. HCl solution. The organic phase was washed with brine, dried (MgSO₄) and concentrated in vacuo. Purification by reverse phase automated flash chromatography at pH 4 (CombiFlash Rf, C18 30 g Gold RediSep column) eluting with a gradient of 10-100% MeCN in water afforded Example 46 as a white solid (156 mg, 0.24 mmol, 80%). LRMS calculated for C₃₅H₄₁Cl₂N₃O₄: 637; found: 638 (M+H). ¹H NMR (400 MHz, DMSO-d₆) δ ppm: 12.76 (br s, 1H), 8.12 (d, J=5.6 Hz, 1H), 7.36 (s, 1H), 7.23 (s, 1H), 7.08 (t, J=7.9 Hz, 1H), 6.74 (d, J=5.6 Hz, 1H), 6.61-6.55 (m, 2H), 6.53-6.48 (m, 1H), 6.29 (br s, 1H), 4.04-3.93 (m, 2H), 3.91 (d, J=12.8 Hz, 1H), 3.85 (s, 3H), 3.83 (d, J=12.8 Hz, 1H), 3.07-2.98 (m, 1H), 2.80-2.70 (m, 2H), 2.65-2.53 (m, 2H), 2.38-1.98 (m, 6H), 1.92-1.71 (m, 2H), 1.70-1.60 (m, 1H), 1.55-1.42 (m, 2H), 1.43-1.35 (m, 1H), 1.29-1.21 (m, 1H), 1.11 (d, J=6.8 Hz, 3H), 1.08 (d, J=6.7 Hz, 3H).

### Example 47 (1s,4S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

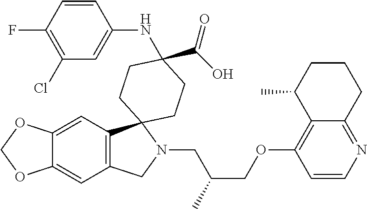 [SMILES: C[C@@H](COc1ccnc2c1[C@H](C)CCC2)CN1Cc2cc3c(cc2[C@]12CC[C@@](Nc1ccc(F)c(Cl)c1)(C(=O)O)CC2)OCO3]

and

### Example 48 (1r,4R)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

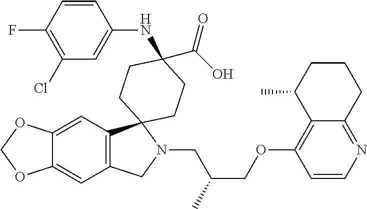 [SMILES: C[C@@H](COc1ccnc2c1[C@H](C)CCC2)CN1Cc2cc3c(cc2[C@]12CC[C@](Nc1ccc(F)c(Cl)c1)(C(=O)O)CC2)OCO3]

Using General procedure 16 and Example 441 as the appropriate amino acid and 2-chloro-1-fluoro-4-iodo-benzene as the appropriate iodobenzene a mixture of diastereoisomers was obtained. The crude intermediate was purified via flash chromatography using DCM and MeOH (1.2% NH₃) as eluents to give a mixture of diastereoisomers. The diastereoisomers were separated via prep RP-HPLC using 25 mM aq. NH₄HCO₃ solution and MeCN as eluents. The diastereoisomer eluting earlier was collected as Example 47. ¹H NMR (500 MHz, DMSO-d₆) δ: 8.1 (d, 1H), 7.06 (t, 1H), 7.02 (s, 1H), 6.84 (s, 1H), 6.72 (d, 1H), 6.64 (dd, 1H), 6.49 (ddd, 1H), 6.02 (br s, 1H), 5.98 (s, 2H), 3.98/3.94 (dd+dd, 2H), 3.85/3.77 (d+d, 2H), 3 (m, 1H), 2.74/2.52 (dd+dd, 2H), 2.72/2.6 (m+m, 2H), 2.24-1.14 (m, 8H), 2.11 (m, 1H), 1.76/1.64 (m+m, 2H), 1.5/1.44 (m+m, 2H), 1.1 (d, 3H), 1.06 (d, 3H). HRMS calculated for C₃₅H₃₉ClFN₃O₅: 635.2562; found 636.2616 (M+H).

The diastereoisomer eluting later was collected as Example 48. ¹H NMR (500 MHz, DMSO-d₆) δ: 8.11 (d, 1H), 7.11 (t, 1H), 6.95 (s, 1H), 6.83 (s, 1H), 6.74 (d, 1H), 6.68 (dd, 1H), 6.56 (dt, 1H), 6.11 (br, 1H), 5.96 (m, 2H), 4/3.96 (dd+dd, 2H), 3.89/3.71 (d+d, 2H), 3.27 (m, 1H), 2.77/2.66 (dm+m, 2H), 2.72/2.42 (dd+dd, 2H), 2.36-1.3 (m, 12H), 2.1 (br, 1H), 1.17 (d, 3H), 1.07 (d, 3H). HRMS calculated for C₃₅H₃₉ClFN₃O₅: 635.2562; found 636.2640 (M+H).

### Example 49 (1s,4S)-4-(3-chloro-2-methylanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

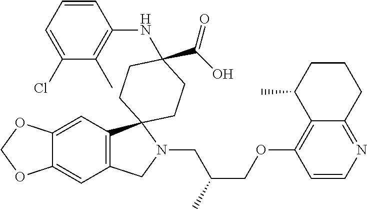 [SMILES: Cc1c(Cl)cccc1N[C@]1(C(=O)O)CC[C@]2(CC1)c1cc3c(cc1CN2C[C@@H](C)COc1ccnc2c1[C@H](C)CCC2)OCO3]

### Example 50 (1r,4R)-4-(3-chloro-2-methylanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid

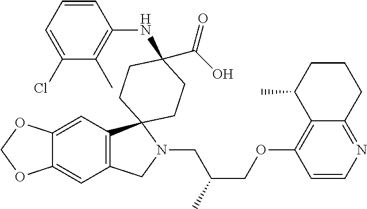 [SMILES: Cc1c(Cl)cccc1N[C@]1(C(=O)O)CC[C@@]2(CC1)c1cc3c(cc1CN2C[C@@H](C)COc1ccnc2c1[C@H](C)CCC2)OCO3]

Using General procedure 16 and Example 441 as the appropriate amino acid and 1-chloro-3-iodo-2-methyl-benzene as the appropriate iodobenzene a mixture of diastereoisomers was obtained. The crude intermediate was purified via flash chromatography using DCM and MeOH (1.2% NH₃) as eluents to give a mixture of diastereoisomers. The diastereoisomers were separated via prep RP-HPLC using 25 mM aq. NH₄HCO₃ solution and MeCN as eluents. The diastereoisomer eluting earlier was collected as Example 49. ¹H NMR (500 MHz, DMSO-d₆) δ: 8.09 (d, 1H), 7.01 (s, 1H), 6.96 (t, 1H), 6.85 (s, 1H), 6.73 (d, 1H), 6.72 (d, 1H), 6.3 (d, 1H), 5.98 (m, 2H), 4.96 (br, 1H), 4.02/3.89 (dd+dd, 2H), 3.89/3.72 (d+d, 2H), 2.92 (m, 1H), 2.77/2.49 (dd+dd, 2H), 2.71/2.59 (dm+m, 2H), 2.31 (s, 3H), 2.27-1.09 (m, 12H), 2.12 (br, 1H), 1.09 (d, 3H), 1.07 (d, 3H). HRMS calculated for C₃₆H₄₂ClN₃O₅: 631.2813; found 632.2878 (M+H).

The diastereoisomer eluting later was collected as Example 50. ¹H NMR (500 MHz, DMSO-d₆) δ: 8.12 (d, 1H), 6.98 (s, 1H), 6.93 (t, 1H), 6.82 (s, 1H), 6.75 (d, 1H), 6.68 (d, 1H), 6.51 (br d, 1H), 5.96 (m, 2H), 4.98 (br, 1H), 3.99 (m, 2H), 3.88/3.73 (d+d, 2H), 3.24 (m, 1H), 2.77/2.66 (dm+m, 2H), 2.73/2.44 (dd+dd, 2H), 2.39-1.34 (m, 12H), 2.25 (s, 3H), 2.11 (m, 1H), 1.17 (d, 3H), 1.08 (d, 3H). HRMS calculated for C₃₆H₄₂ClN₃O₅: 631.2813; found 632.2877 (M+H).

### Example 51

### Example 51A methyl 2-bromo-5-fluoro-4-methylbenzoate

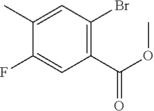

To a solution of 2-bromo-5-fluoro-4-methylbenzoic acid (12.5 g, 53.6 mmol, 1 eq) in MeOH (250 mL) was added cc. H₂SO₄ (20 mL) and the mixture was heated at reflux for 18 h. The mixture was concentrated in vacuo, the residue was cooled to 0° C., diluted with EtOAc and 2 M aq. NaOH solution (200 mL) with stirring. The organic phase was washed with brine, dried (MgSO₄) and concentrated in vacuo to afford Example 51A as a clear oil (13 g, 52.7 mmol, 98%). ¹H NMR (400 MHz, DMSO-d₆) δ ppm: 7.76-7.73 (m, 1H), 7.60 (d, J=9.8 Hz, 1H), 3.85 (s, 3H), 2.28 (dd, J=2.1, 0.8 Hz, 3H).

### Example 51B methyl 2-bromo-4-(bromomethyl)-5-fluorobenzoate

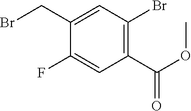

To a solution of Example 51A (13 g, 52.7 mmol, 1 eq) in CCl₄ (190 mL) was added NBS (9.39 g, 52.7 mmol, 1 eq), followed by benzoyl peroxide (0.55 mL, 2.64 mmol, 0.05 eq) and the mixture was heated at 80° C. for 5 h. The mixture was allowed to cool to rt, partitioned between DCM and 2 N aq. NaOH solution. The organic phase was washed with brine, dried (MgSO₄) and concentrated in vacuo. Purification by automated flash chromatography (CombiFlash Rf, 330 g RediSep™ silica cartridge) eluting with a gradient of 0-5% EtOAc in heptane afforded Example 51B as a clear oil (16.6 g, 50.8 mmol, 96%). ¹H NMR (400 MHz, DMSO-d₆) δ ppm: 8.01 (d, J=6.8 Hz, 1H), 7.71 (d, J=10.0 Hz, 1H), 4.71 (s, 2H), 3.87 (s, 3H).

### Example 51C methyl 2-bromo-5-fluoro-4-(methoxymethyl)benzoate

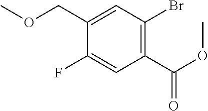

Na (1.63 g, 71.1 mmol, 1.4 eq) was dissolved in MeOH (40 mL), then Example 51B (16.6 g, 50.8 mmol, 1 eq) in MeOH (80 mL) was added and the mixture was heated at 50° C. for 1 h. The mixture was partitioned between EtOAc and water, and the organic phase was washed with brine, dried (MgSO₄) and concentrated in vacuo. Purification by automated flash chromatography (CombiFlash Rf, 330 g RediSep™ silica cartridge) eluting with a gradient of 0-5% EtOAc in heptane afforded Example 51C as colourless crystals (3.1 g, 11.2 mmol, 22%). ¹H NMR (400 MHz, DMSO-d₆) δ ppm: 7.80-7.77 (m, 1H), 7.67 (d, J=10.0 Hz, 1H), 4.51 (t, J=1.0 Hz, 2H), 3.87 (s, 3H), 3.34 (s, 3H).

### Example 51D 2-bromo-5-fluoro-4-(methoxymethyl)benzoic acid

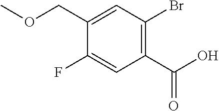

To a solution of Example 51C (3.1 g, 11.2 mmol, 1 eq) in THE (35 mL) was added 2 M aq. NaOH solution (16.8 mL, 33.6 mmol, 3 eq) and the mixture was heated at 50° C. for 18 h. The mixture was concentrated in vacuo, acidified with 2 N aq. HCl solution (20 mL) and the resultant solid was collected by filtration, washed with water and dried under vacuum to afford Example 51D as a white solid (2.78 g, 10.6 mmol, 95%). ¹H NMR (400 MHz, DMSO-d₆) δ ppm: 13.70 (s, 1H), 7.74 (d, J=6.5 Hz, 1H), 7.61 (d, J=10.0 Hz, 1H), 4.50 (t, J=0.9 Hz, 2H), 3.34 (s, 3H).

### Example 51E 2-bromo-5-fluoro-4-(methoxymethyl)benzoyl chloride

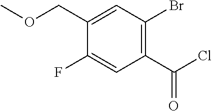

To a solution of Example 51D (2.78 g, 10.6 mmol, 1 eq) in DCM (50 mL) and DMF (20 μL), cooled to 0° C. was added oxalyl chloride (26.4 mL, 2 M in DCM, 52.8 mmol, 5 eq) and the mixture was stirred for 1 h. The mixture was concentrated in vacuo to afford Example 51E as a yellow solid (3.03 g, 10.8 mmol, quant.) that was used directly in the subsequent step without further purification. ¹H NMR (400 MHz, DMSO-d₆) δ ppm: 7.74 (d, J=6.6 Hz, 1H), 7.62 (d, J=10.0 Hz, 1H), 4.50 (s, 2H), 3.34 (s, 3H).

### Example 51F 2-bromo-N-(1,4-dioxaspiro[4.5]dec-7-en-8-yl)-5-fluoro-4-(methoxymethyl)-N-{(2R)-3-[(4-methoxyphenyl)methoxy]-2-methylpropyl}benzamide

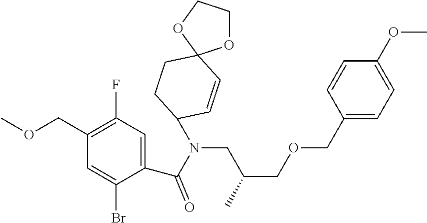

To a solution of Preparation 3e (2.05 g, 9.78 mmol, 1 eq) in toluene (200 mL) was added 1,4-dioxaspiro[4.5]decan-8-one (1.53 g, 9.78 mmol, 1 eq) and the mixture was heated at 125° C. under Dean-Stark reflux for 2.5 h. The mixture was allowed to cool to rt and concentrated in vacuo. The residue was dissolved in DCM (50 mL), TEA (2.59 mL, 18.6 mmol, 1.9 eq) was added followed by Example 51E (3.03 g, 10.8 mmol, 1.1 eq) in DCM (18 mL). The mixture was stirred at rt for 30 min, then partitioned between DCM and sat. aq. NH₄Cl solution, and the organic phase was washed with brine, dried (PTFE phase separator) and concentrated in vacuo. Purification by automated flash chromatography (CombiFlash Rf, 80 g RediSep™ silica cartridge) eluting with a gradient of 0-48% EtOAc in heptane afforded Example 51F as a yellow oil (3.77 g, 6.36 mmol, 65%). LRMS calculated for C₂₉H₃₅BrFNO₆: 591; found: 592 (M+H). ¹H NMR (400 MHz, DMSO-d₆) δ ppm: 7.61 (d, J=6.5 Hz, 1H), 7.29-7.24 (m, 3H), 6.94-6.89 (m, 2H), 5.44-5.40 (m, 1H), 4.45 (s, 2H), 4.43 (d, J=11.8 Hz, 1H), 4.37 (d, J=11.8 Hz, 1H), 3.82-3.71 (m, 7H), 3.62-3.36 (m, 3H), 3.33-3.25 (m, 4H), 2.29-2.16 (m, 2H), 2.05-1.95 (m, 1H), 1.96-1.85 (m, 2H), 1.55-1.48 (m, 2H), 0.95 (d, J=6.7 Hz, 3H).

### Example 51G 5″-fluoro-6″-(methoxymethyl)-2″-{(2R)-3-[(4-methoxyphenyl)methoxy]-2-methylpropyl}dispiro[[1,3]dioxolane-2,1′-cyclohex[2]ene-4′,1″-isoindol]-3″(2″H)-one

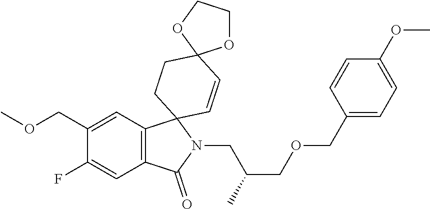

To an oven-dried sealed as was added Example 51F (4.03 g, 6.8 mmol, 1 eq) in MeCN (70 mL) followed by PPh₃ (892 mg, 3.4 mmol, 0.5 eq), TBACl (1.49 mL, 10.2 mmol, 1.5 eq), K₂CO₃ (2.82 g, 20.4 mmol, 3 eq) and Pd(OAc)₂ (229 mg, 1.02 mmol, 0.15 eq). The mixture was heated at 120° C. in a sealed flask for 24 h. The mixture was allowed to cool to rt, filtered through celite and partitioned between EtOAc and sat. aq. NaHCO₃ solution, and the organic phase was washed with brine, dried (MgSO₄) and concentrated in vacuo. Purification by automated flash chromatography (CombiFlash Rf, 80 g RediSep™ silica cartridge) eluting with a gradient of 0-60% EtOAc in heptane afforded a 1:1 mixture of diastereoisomers, Example 51G as a yellow oil (2.87 g, 5.61 mmol, 83%). LRMS calculated for C₂₉H₃₄FNO₆: 511; found: 512 (M+H). ¹H NMR (400 MHz, DMSO-d₆) δ ppm: 7.52-7.44 (m, 2H), 7.28-7.19 (m, 2H), 6.91-6.84 (m, 2H), 6.01/5.98 (d, 1H), 5.43/5.38 (d, 1H), 4.56 (s, 2H), 4.44-4.29 (m, 2H), 4.07-3.87 (m, 4H), 3.74/3.73 (s, 3H), 3.44-3.13 (m, 7H), 2.39-2.27 (m, 2H), 2.21-2.11 (m, 1H), 2.07-1.99 (m, 1H), 1.87-1.75 (m, 1H), 0.90/0.86 (d, 3H).

### Example 51H 5″-fluoro-6″-(methoxymethyl)-2″-{(2R)-3-[(4-methoxyphenyl)methoxy]-2-methylpropyl}dispiro[[1,3]dioxolane-2,1′-cyclohexane-4′,1″-isoindol]-3″(2″H)-one

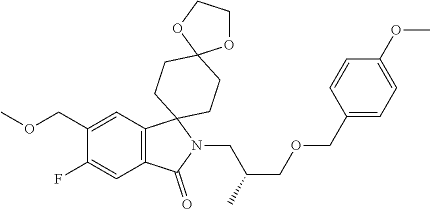

To a solution of Example 51G (2.87 g, 5.61 mmol, 1 eq) in EtOH (75 mL) was added HCO₂NH₄ (1.77 g, 28.1 mmol, 5 eq) and the mixture was sparged with N₂ (10 min). Pd/C (597 mg, 0.56 mmol, 0.1 eq) was added, the mixture was sparged with N₂ (10 min) and heated at reflux for 2 h. The mixture was diluted with EtOAc, filtered through celite and the filtrate was partitioned between EtOAc and water, and the organic phase was washed with brine, dried (MgSO₄) and concentrated in vacuo. Purification by automated flash chromatography (CombiFlash Rf, 40 g RediSep™ silica cartridge) eluting with a gradient of 0-60% EtOAc in heptane afforded Example 51H as a yellow oil (2.27 g, 4.42 mmol, 79%). LRMS calculated for C₂₉H₃₆FNO₆: 513; found: 514 (M+H). ¹H NMR (400 MHz, DMSO-d₆) δ ppm: 7.87 (d, J=5.9 Hz, 1H), 7.48 (d, J=8.7 Hz, 1H), 7.27-7.21 (m, 2H), 6.92-6.86 (m, 2H), 4.60 (s, 2H), 4.40-4.32 (m, 2H), 4.01-3.91 (m, 4H), 3.74 (s, 3H), 3.38 (s, 3H), 3.37-3.29 (m, 3H), 3.20 (dd, J=14.1, 7.7 Hz, 1H), 2.50-2.44 (m, 1H), 2.33-2.18 (m, 2H), 2.14-2.02 (m, 2H), 1.91-1.81 (m, 2H), 1.44-1.31 (m, 2H), 0.90 (d, J=6.8 Hz, 3H).

### Example 511 5″-fluoro-6″-(methoxymethyl)-2″-{(2R)-3-[(4-methoxyphenyl)methoxy]-2-methylpropyl}-2″,3″-dihydrodispiro[[1,3]dioxolane-2,1′-cyclohexane-4′,1″-isoindole]

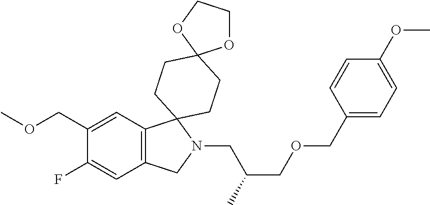

To a solution of Example 51H (2.27 g, 4.42 mmol, 1 eq) in THE (45 mL), cooled to 0° C. was added LAH (6.63 mL, 1 M, 6.63 mmol, 1.5 eq) and the mixture was stirred at 0° C. for 1 h then warmed to rt for 2 h. The mixture was cooled to 0° C., water (252 μL) was added followed by 15% aq. NaOH solution (252 μL) then water (746 μL) and the mixture was stirred for 30 min. EtOAc and MgSO₄ were added to the mixture, stirred for 10 min, filtered through celite and concentrated in vacuo. Purification by automated flash chromatography (CombiFlash Rf, 40 g RediSep™ silica cartridge) eluting with a gradient of 0-40% EtOAc in heptane afforded Example 511 as a clear oil (2.02 g, 4.04 mmol, 92%). LRMS calculated for C₂₉H₃₈FNO₅: 499; found: 500 (M+H). ¹H NMR (400 MHz, DMSO-d₆) δ ppm: 7.50 (d, J=6.6 Hz, 1H), 7.27-7.22 (m, 2H), 7.11 (d, J=9.6 Hz, 1H), 6.93-6.87 (m, 2H), 4.46 (s, 2H), 4.40-4.33 (m, 2H), 3.99-3.87 (m, 4H), 3.88-3.80 (m, 2H), 3.75 (s, 3H), 3.38 (dd, J=9.0, 4.9 Hz, 1H), 3.33-3.28 (m, 4H), 2.60 (dd, J=11.9, 7.3 Hz, 1H), 2.34 (dd, J=12.2, 6.9 Hz, 1H), 1.97-1.81 (m, 5H), 1.79-1.67 (m, 2H), 1.52-1.40 (m, 2H), 0.91 (d, J=6.8 Hz, 3H).

### Example 51J=5′-fluoro-2′-[(2R)-3-hydroxy-2-methylpropyl]-6′-(methoxymethyl)-2′,3′-dihydrospiro[cyclohexane-1,1′-isoindol]-4-one

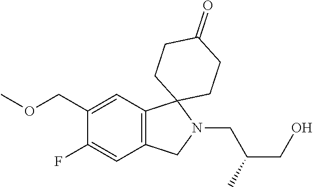 [SMILES: COCc1cc2c(cc1F)CN(C[C@@H](C)CO)C21CCC(=O)CC1]

To a solution of Example 511 (2.02 g, 4.04 mmol, 1 eq) in THE (45 mL) was added PTSA (1.21 mL, 12.1 mmol, 3 eq) in water (45 mL) and the mixture was heated at 85° C. for 72 h. The mixture was allowed to cool to rt and partitioned between EtOAc and sat. aq. NaHCO₃ solution. The combined organic phase was washed with brine, dried (MgSO₄) and concentrated in vacuo. Purification by automated flash chromatography (CombiFlash Rf, 40 g RediSep™ silica cartridge) eluting with a gradient of 0-90% EtOAc in heptane afforded Example 51J as a peach gum (915 mg, 2.73 mmol, 68%). LRMS calculated for C₁₉H₂₆FNO₃: 335; found: 336 (M+H). ¹H NMR (400 MHz, DMSO-d₆) δ ppm: 7.60 (d, J=6.6 Hz, 1H), 7.16 (d, J=9.8 Hz, 1H), 4.49 (dd, J=5.7, 4.7 Hz, 1H), 4.45 (s, 2H), 3.96-3.86 (m, 2H), 3.41 (dt, J=10.2, 5.0 Hz, 1H), 3.34-3.27 (m, 1H), 3.29 (s, 3H), 2.72-2.58 (m, 3H), 2.46-2.35 (m, 3H), 2.18-2.07 (m, 2H), 1.84-1.71 (m, 3H), 0.87 (d, J=6.7 Hz, 3H).

### Example 51K (1'S,1″s)-5″-fluoro-2″-[(2R)-3-hydroxy-2-methylpropyl]-6″-(methoxymethyl)-2″,3″-dihydrodispiro[imidazolidine-4,1′-cyclohexane-4′,1″-isoindole]-2,5-dione

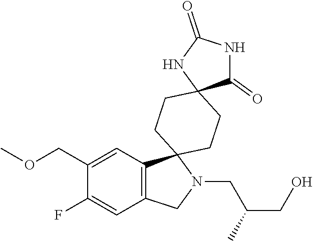 [SMILES: COCc1cc2c(cc1F)CN(C[C@@H](C)CO)[C@]21CC[C@]2(CC1)NC(=O)NC2=O]

To a solution of Example 51J (915 mg, 2.73 mmol, 1 eq) in a mixture of EtOH (11 mL) and water (11 mL) was added NaCN (267 mg, 5.46 mmol, 2 eq) and (NH₄)₂CO₃ (1.05 g, 10.9 mmol, 4 eq). The mixture was heated at 60° C. for 24 h. A mixture of diastereoisomers was obtained. The mixture was cooled to rt, then cooled to 0° C. and a precipitate formed. The solids were collected by filtration, washed with water and dried under vacuum to afford a single diastereoisomer Example 51K as a white solid (535 mg, 1.32 mmol, 48%). LRMS calculated for C₂₁H₂₈FN₃O₄: 405; found: 406 (M+H). ¹H NMR (400 MHz, DMSO-d₆) δ ppm: 10.71 (s, 1H), 8.86 (s, 1H), 7.54 (d, J=6.7 Hz, 1H), 7.16 (d, J=9.7 Hz, 1H), 4.55 (dd, J=5.9, 4.4 Hz, 1H), 4.46 (s, 2H), 3.94 (d, J=13.6 Hz, 1H), 3.87 (d, J=13.6 Hz, 1H), 3.45 (dt, J=10.2, 5.2 Hz, 1H), 3.32 (s, 3H), 3.26 (dt, J=10.2, 6.2 Hz, 1H), 2.80 (dd, J=12.4, 7.4 Hz, 1H), 2.59-2.52 (m, 1H), 2.24-2.09 (m, 2H), 1.94-1.82 (m, 2H), 1.82-1.71 (m, 1H), 1.69-1.59 (m, 2H), 1.52-1.42 (m, 2H), 0.88 (d, J=6.7 Hz, 3H).

### Example 51L (1s,4S)-4-amino-5′-fluoro-2′-[(2R)-3-hydroxy-2-methylpropyl]-6′-(methoxymethyl)-2′,3′-dihydrospiro[cyclohexane-1,1′-isoindole]-4-carboxylic acid

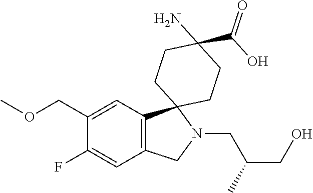 [SMILES: COCc1cc2c(cc1F)CN(C[C@@H](C)CO)[C@]21CC[C@](N)(C(=O)O)CC1]

To a solution of Example 51K (535 mg, 1.32 mmol, 1 eq) in water (5.2 mL) was added LiOH×H₂O (554 mg, 13.2 mmol, 10 eq) and the mixture was heated in a sealed tube at 140° C. for 16 h. The mixture was allowed to cool to 0° C., diluted with water, neutralised with 2 M aq. HCl solution (6.5 mL) and partitioned between iPrOH/DCM (1:4) and water. The organic phase was washed with brine, dried (MgSO₄) and concentrated in vacuo to afford Example 51L as an off-white solid (352 mg, 0.93 mmol, 70%). LRMS calculated for C₂₀H₂₉FN₂O₄: 380; found: 381 (M+H). ¹H NMR (400 MHz, DMSO-d₆) δ ppm: 7.78 (d, J=6.7 Hz, 1H), 7.69 (br s, 2H), 7.13 (d, J=9.7 Hz, 1H), 4.57 (br s, 1H), 4.41 (s, 2H), 3.94 (d, J=13.6 Hz, 1H), 3.87 (d, J=13.6 Hz, 1H), 3.44 (dd, J=10.4, 5.9 Hz, 1H), 3.30 (s, 3H), 3.30-3.22 (m, 1H), 2.77 (dd, J=12.2, 7.4 Hz, 1H), 2.51-2.38 (m, 3H), 1.86-1.62 (m, 5H), 1.43-1.33 (m, 2H), 0.87 (d, J=6.6 Hz, 3H).

### Example 51M (1s,4S)-4-(3-chloroanilino)-5′-fluoro-2′-[(2R)-3-hydroxy-2-methylpropyl]-6′-(methoxymethyl)-2′,3′-dihydrospiro[cyclohexane-1,1′-isoindole]-4-carboxylic acid

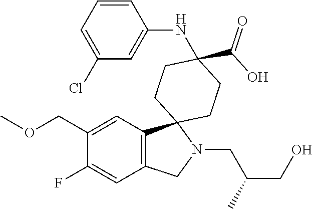 [SMILES: COCc1cc2c(cc1F)CN(C[C@@H](C)CO)[C@]21CC[C@](Nc2cccc(Cl)c2)(C(=O)O)CC1]

To a solution of Example 51L (352 mg, 0.93 mmol, 1 eq) in DMF (7 mL) was added Cs₂CO₃ (603 mg, 1.85 mmol, 2 eq), followed by 3-chloroiodobenzene (126 μL, 1.02 mmol, 1.1 eq) and ethyl 2-cyclohexanonecarboxylate (59 μL, 0.37 mmol, 0.4 eq). The mixture was sparged with N₂ (10 min) before the addition of CuI (18 mg, 0.09 mmol, 0.1 eq) and the mixture was heated at 140° C. for 1 h under microwave irradiation. The mixture was cooled to rt, filtered through celite, the solids were washed with DCM and the filtrate was concentrated in vacuo. The residue was dissolved in DCM, then loaded onto a DCM-wet PE-AX cartridge (10 g), washed successively with DCM and MeOH, eluted with 15% HCOOH in DCM, and concentrated in vacuo to afford Example 51M as a brown gum (179 mg, 0.36 mmol, 39%). LRMS calculated for C₂₆H₃₂ClFN₂O₄: 490; found: 491 (M+H). ¹H NMR (400 MHz, DMSO-d₆) δ ppm: 7.54 (d, J=6.6 Hz, 1H), 7.14 (d, J=9.7 Hz, 1H), 7.08 (t, J=8.1 Hz, 1H), 6.62 (t, J=2.1 Hz, 1H), 6.59-6.52 (m, 2H), 6.27 (br s, 1H), 4.47 (s, 2H), 3.93-3.83 (m, 2H), 3.43 (dd, J=10.3, 5.7 Hz, 1H), 3.32 (s, 3H), 3.27 (dd, J=10.3, 6.0 Hz, 1H), 2.61 (dd, J=12.2, 7.5 Hz, 1H), 2.41 (dd, J=11.8, 7.1 Hz, 1H), 2.23-2.07 (m, 4H), 2.05-1.89 (m, 2H), 1.82-1.72 (m, 1H), 1.37-1.25 (m, 2H), 0.86 (d, J=6.7 Hz, 3H).

### Example 51N methyl (1s,4S)-4-(3-chloroanilino)-5′-fluoro-2′-[(2R)-3-hydroxy-2-methylpropyl]-6′-(methoxymethyl)-2′,3′-dihydrospiro[cyclohexane-1,1′-isoindole]-4-carboxylate

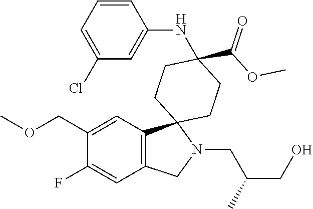 [SMILES: COCc1cc2c(cc1F)CN(C[C@@H](C)CO)[C@]21CC[C@](Nc2cccc(Cl)c2)(C(=O)OC)CC1]

To a solution of Example 51M (179 mg, 0.36 mmol, 1 eq) in DCM (3 mL) and MeOH (3 mL), cooled to 0° C., was added TMS-CHNN (1.82 mL, 0.6 M, 1.09 mmol, 3 eq) dropwise. The mixture was stirred for 1 h, then further TMS-CHNN (33.3 mL, 0.6 M, 20 mmol, 2 eq) was added and stirred for 1 h at 0° C. and at rt for 1.5 h. The mixture was quenched with the slow addition of sat. aq. NaHCO₃ solution, partitioned between DCM and water, the aq. phase was extracted with DCM, and the combined organic extracts were washed with brine, dried (PTFE phase separator) and concentrated in vacuo. Purification by automated flash chromatography (CombiFlash Rf, 40 g RediSep™ silica cartridge) eluting with a gradient of 0-60% EtOAc in heptane afforded Example 51N as a clear gum (67 mg, 0.13 mmol, 36%). LRMS calculated for C₂₇H₃₄ClFN₂O₄: 504; found: 505 (M+H). ¹H NMR (400 MHz, DMSO-d₆) δ ppm: 7.54 (d, J=6.5 Hz, 1H), 7.15 (d, J=9.7 Hz, 1H), 7.09 (t, J=8.0 Hz, 1H), 6.63-6.57 (m, 2H), 6.45 (dd, J=7.9, 2.2 Hz, 1H), 6.35 (s, 1H), 4.55 (dd, J=5.8, 4.4 Hz, 1H), 4.48 (s, 2H), 3.92-3.83 (m, 2H), 3.66 (s, 3H), 3.46-3.37 (m, 1H), 3.31-3.22 (m, 1H), 2.61 (dd, J=11.9, 7.2 Hz, 1H), 2.40 (dd, J=12.0, 7.2 Hz, 1H), 2.25-2.10 (m, 4H), 2.03-1.90 (m, 2H), 1.82-1.72 (m, 1H), 1.37-1.28 (m, 2H), 0.86 (d, J=6.7 Hz, 3H).

### Example 510 methyl (1s,4S)-4-(3-chloroanilino)-5′-fluoro-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

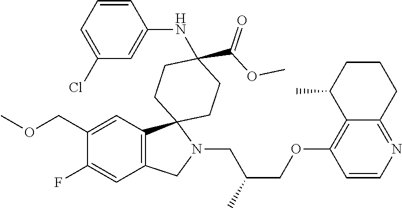 [SMILES: COCc1cc2c(cc1F)CN(C[C@@H](C)COc1ccnc3c1[C@H](C)CCC3)[C@]21CC[C@](Nc2cccc(Cl)c2)(C(=O)OC)CC1]

To a solution of Example 51N (67 mg, 0.13 mmol, 1 eq) in THE (4 mL) was added Preparation 2a1 (43 mg, 0.27 mmol, 2 eq) and PPh₃ (70 mg, 0.27 mmol, 2 eq), followed by DTBAD (61 mg, 0.27 mmol, 2 eq) and the mixture was stirred at 50° C. for 6 h. The mixture was partitioned between EtOAc and sat. aq. NaHCO₃ solution, and the organic phase was washed with brine, dried (MgSO₄) and concentrated in vacuo. Purification by automated flash chromatography (CombiFlash Rf, 4 g RediSep™ silica cartridge) eluting with a gradient of 0-90% EtOAc in heptane afforded a solid. The solid was dissolved in MeOH, then loaded onto a MeOH-wet SCX cartridge (5 g), washed successively with DCM and MeOH, eluted with 7 M NH₃ in MeOH/MeOH (1:4), and concentrated in vacuo to afford Example 510 as a white solid (44 mg, 0.07 mmol, 51%). LRMS calculated for C₃₇H₄₅ClFN₃O₄: 649; found: 650 (M+H). ¹H NMR (400 MHz, DMSO-d₆) δ ppm: 8.12 (d, J=5.6 Hz, 1H), 7.54 (d, J=6.7 Hz, 1H), 7.15 (d, J=9.7 Hz, 1H), 7.11-7.04 (m, 1H), 6.74 (d, J=5.7 Hz, 1H), 6.63-6.56 (m, 2H), 6.46-6.39 (m, 1H), 6.35 (s, 1H), 4.47 (s, 2H), 4.04-3.87 (m, 4H), 3.65 (s, 3H), 3.33 (s, 3H), 3.08-2.98 (m, 1H), 2.81-2.69 (m, 2H), 2.67-2.53 (m, 2H), 2.31-1.99 (m, 6H), 1.95-1.60 (m, 3H), 1.57-1.43 (m, 2H), 1.41-1.32 (m, 1H), 1.29-1.21 (m, 1H), 1.12 (d, J=6.9 Hz, 3H), 1.08 (d, J=6.7 Hz, 3H).

### Example 51 (1s,4S)-4-(3-chloroanilino)-5′-fluoro-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

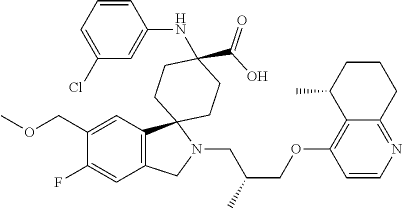 [SMILES: COCc1cc2c(cc1F)CN(C[C@@H](C)COc1ccnc3c1[C@H](C)CCC3)[C@]21CC[C@](Nc2cccc(Cl)c2)(C(=O)O)CC1]

To a solution of Example 510 (66 mg, 0.1 mmol, 1 eq) in 1,4-dioxane (3 mL) and water (0.3 mL) was added LiOH×H₂O (43 mg, 1.02 mmol, 10 eq) and the mixture was heated at reflux for 3 h. The reaction was allowed to cool to rt and purification by reverse phase automated flash chromatography at pH 4 (CombiFlash Rf, C18 13 g RediSep column) eluting with a gradient of 10-100% MeCN in water afforded Example 51 as a white solid (62.1 mg, 0.1 mmol, 96%). HRMS calculated for C₃₆H₄₃N₃O₄FCl: 635.2926; found: 636.2989 (M+H). ¹H NMR (400 MHz, DMSO-d₆) δ ppm: 8.12 (d, J=5.6 Hz, 1H), 7.54 (d, J=6.6 Hz, 1H), 7.14 (d, J=9.7 Hz, 1H), 7.05 (t, J=8.1 Hz, 1H), 6.74 (d, J=5.6 Hz, 1H), 6.61 (t, J=2.1 Hz, 1H), 6.57-6.50 (m, 2H), 6.23 (br s, 1H), 4.45 (s, 2H), 4.05-3.85 (m, 4H), 3.31 (s, 3H), 3.08-2.99 (m, 1H), 2.81-2.69 (m, 2H), 2.67-2.52 (m, 2H), 2.28-1.97 (m, 6H), 1.94-1.62 (m, 3H), 1.57-1.44 (m, 2H), 1.38-1.28 (m, 1H), 1.27-1.18 (m, 1H), 1.12 (d, J=6.8 Hz, 3H), 1.08 (d, J=6.7 Hz, 3H).

### Example 52

### Example 52A (1s,4S)-4-(3-chloro-2-methylanilino)-5′-fluoro-2′-[(2R)-3-hydroxy-2-methylpropyl]-2′,3′-dihydrospiro[cyclohexane-1,1′-isoindole]-4-carboxylic acid

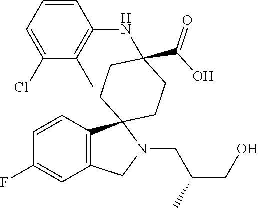 [SMILES: Cc1c(Cl)cccc1N[C@]1(C(=O)O)CC[C@]2(CC1)c1ccc(F)cc1CN2C[C@@H](C)CO]

To a solution of Example 41G (175 mg, 0.52 mmol, 1 eq) in DMF (5 mL) was added Cs₂CO₃ (339 mg, 1.04 mmol, 2 eq), 2-(2-methylpropanoyl)cyclohexan-1-one (0.02 mL, 0.1 mmol, 0.2 eq) and 1-chloro-3-iodo-2-methylbenzene (0.09 mL, 0.62 mmol, 1.2 eq). The mixture was sparged with N₂ (10 min) before the addition of CuI (5 mg, 0.03 mmol, 0.05 eq) and the mixture was heated at 115° C. for 18 h. The mixture was cooled to rt, water (75 mL) was added and the mixture was neutralised with AcOH, then partitioned between EtOAc and water, and the combined organic extracts were washed with brine, dried (MgSO₄) and concentrated in vacuo to afford Example 52A as a dark brown gum (200 mg, 0.43 mmol, 83%) that was used directly in the subsequent step without further purification. LRMS calculated for C₂₅H30N₂O₃FCl: 460; found: 461 (M+H).

### Example 52B methyl (1s,4S)-4-(3-chloro-2-methylanilino)-5′-fluoro-2′-[(2R)-3-hydroxy-2-methylpropyl]-2′,3′-dihydrospiro[cyclohexane-1,1′-isoindole]-4-carboxylate

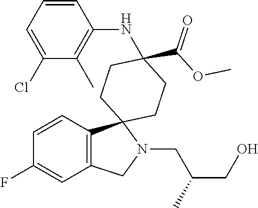 [SMILES: COC(=O)[C@]1(Nc2cccc(Cl)c2C)CC[C@@]2(CC1)c1ccc(F)cc1CN2C[C@@H](C)CO]

To a solution of Example 52A (200 mg, 0.43 mmol, 1 eq) in DCM (5 mL) and MeOH (5 mL), cooled to 0° C., was added TMS-CHNN (1.45 mL, 0.6 M, 0.87 mmol, 2 eq) dropwise and the mixture was stirred for 30 min at 0° C., then warmed to rt for 1.5 h. The mixture was concentrated in vacuo. Purification by flash chromatography (isolute flash silica, 20 g) eluting with a gradient of 0-30% EtOAc in heptane afforded Example 52B as a dark yellow gum (55 mg, 0.12 mmol, 27%). LRMS calculated for C₂₆H₃₂ClFN₂O₃: 474; found: 475 (M+H). ¹H NMR (400 MHz, CDCl₃) δ ppm: 7.51 (dd, J=9.2, 4.8 Hz, 1H), 7.00-6.92 (m, 3H), 6.85 (dd, J=8.0, 1.0 Hz, 1H), 6.31 (dd, J=8.1, 1.1 Hz, 1H), 3.72 (s, 3H), 3.49 (t, J=10.3 Hz, 1H), 2.84 (s, 1H), 2.74 (d, J=11.2 Hz, 1H), 2.55 (td, J=14.2, 5.0 Hz, 1H), 2.36 (s, 3H), 2.33-2.13 (m, 4H), 2.00 (s, 1H), 1.84 (s, 1H), 1.36-1.25 (m, 2H), 0.89 (d, 3H), 0.87-0.78 (m, 3H).

### Example 52C methyl (1s,4S)-4-(3-chloro-2-methylanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

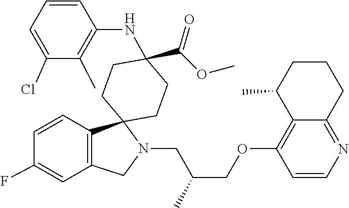 [SMILES: COC(=O)[C@]1(Nc2cccc(Cl)c2C)CC[C@@]2(CC1)c1ccc(F)cc1CN2C[C@@H](C)COc1ccnc2c1[C@H](C)CCC2]

To a solution of Example 52B (50 mg, 0.11 mmol, 1 eq) in toluene (5 mL) was added Preparation 2a1 (34 mg, 0.21 mmol, 2 eq) and PPh₃ (55 mg, 0.21 mmol, 2 eq), followed by DIAD (49 mg, 0.21 mmol, 2 eq). The mixture was stirred at rt for 2 h. The mixture was concentrated in vacuo. Purification by flash chromatography (isolute flash silica, 20 g) eluting with a gradient of 0-40% EtOAc in heptane afforded Example 52C as a pale brown gum (40 mg, 0.06 mmol, 61%). LRMS calculated for C₃₆H₄₃ClFN₃O₃: 619; found: 620 (M+H).

### Example 52 (1s,4S)-4-(3-chloro-2-methylanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

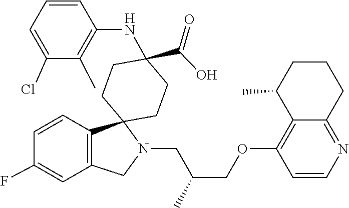 [SMILES: Cc1c(Cl)cccc1N[C@]1(C(=O)O)CC[C@]2(CC1)c1ccc(F)cc1CN2C[C@@H](C)COc1ccnc2c1[C@H](C)CCC2]

To a solution of Example 52C (40 mg, 0.06 mmol, 1 eq) in MeOH (0.75 mL) and water (0.75 mL) was added LiOH×H₂O (8 mg, 0.19 mmol, 3 eq) and the mixture was stirred at 85° C. for 18 h. The mixture was allowed to cool to rt, then acidified to pH 5 with AcOH and the resulting suspension was concentrated in vacuo. Purification by flash chromatography (isolute flash silica, 5 g) eluting with a gradient of 0-5% MeOH in DCM afforded Example 52 as a white solid (9.8 mg, 0.02 mmol, 25%). LRMS calculated for C₃₅H₄₁ClFN₃O₃: 605; found: 606 (M+H). ¹H NMR (400 MHz, DMSO-d₆) δ ppm: 11.97 (br s, 1H), 8.10 (d, J=5.5 Hz, 1H), 7.53 (dd, J=8.7, 5.1 Hz, 1H), 7.14 (dd, J=8.9, 2.5 Hz, 1H), 7.06 (t, J=9.2 Hz, 1H), 6.95 (t, J=8.2 Hz, 1H), 6.77-6.66 (m, 2H), 6.38 (d, J=8.0 Hz, 1H), 4.85 (br s, 1H), 4.07-3.96 (m, 2H), 3.90 (dd, J=9.4, 3.5 Hz, 1H), 3.83 (d, J=13.4 Hz, 1H), 2.98-2.88 (m, 1H), 2.87-2.66 (m, 2H), 2.65-2.53 (m, 2H), 2.32 (s, 3H), 2.31-1.95 (m, 6H), 1.88-1.54 (m, 3H), 1.48-1.35 (m, 2H), 1.32-1.22 (m, 1H), 1.17-1.05 (m, 7H).

### Example 53

### Example 53A 2-bromo-5-methylbenzoyl chloride

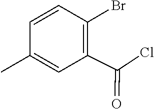

To a solution of 2-bromo-5-methylbenzoic acid (45.0 g, 209 mmol, 1 eq) in DCM (900 mL) and DMF (2 mL), cooled to 0° C., was added oxalyl chloride (125 mL, 2 M in DCM, 250 mmol, 1.2 eq) dropwise. The mixture was stirred at rt for 48 h. The mixture was concentrated in vacuo and to the residue was added anhydrous DCM (100 mL) and this was also removed in vacuo (repeated twice) to give Example 53A as a yellow solid (48.9 g, 209 mmol, 100%) that was used directly in the subsequent step without further purification.

### Example 53B 2-bromo-N-(1,4-dioxaspiro[4.5]dec-7-en-8-yl)-N-{(2R)-3-{(4-methoxyphenyl)methoxyl-2-methylpropyl}-5-methylbenzamide

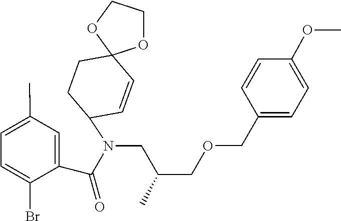 [SMILES: COc1ccc(COC[C@H](C)CN(C(=O)c2cc(C)ccc2Br)C2C=CC3(CC2)OCCO3)cc1]

A solution of 1,4-dioxaspiro[4.5]decan-8-one (8.94 g, 57.2 mmol, 1.09 eq) and Preparation 3e (11 g, 52.6 mmol, 1.0 eq) in CHCl₃ (250 mL) was stirred for 4 h with 4\acute{Å} molecular sieves (10 g). The solids were collected by filtration, washed with DCM and the filtrate was concentrated in vacuo. To a solution of the residue in DCM (250 mL) was added TEA (14.9 mL, 107 mmol, 2.04 eq), cooled to 0° C., followed by the dropwise addition of Example 53A (14.1 g, 60.5 mmol, 1.15 eq) in DCM (50 mL). After complete addition the mixture was stirred at rt for 18 h. The mixture was partitioned between DCM and water, and the organic phase was washed with brine, dried (MgSO₄) and concentrated in vacuo. Purification by automated flash chromatography (CombiFlash Rf, 220 g Gold RediSep™ silica cartridge) eluting with a gradient of 0-35% EtOAc in heptane afforded Example 53B as a yellow oil (17.5 g, 32.2 mmol, 61%). LRMS calculated for C₂₈H₃₄BrNO₅: 543; found: 544 (M+H). ¹H NMR (400 MHz, DMSO-d₆) δ ppm: 7.46-7.42 (m, 1H), 7.30-7.22 (m, 2H), 7.11-7.05 (m, 2H), 6.94-6.89 (m, 2H), 5.38-5.33 (m, 1H), 4.43 (d, J=11.7 Hz, 1H), 4.37 (d, J=11.7 Hz, 1H), 3.82-3.75 (m, 4H), 3.75 (s, 3H), 3.64-3.34 (m, 3H), 3.28 (dd, J=9.5, 6.4 Hz, 1H), 2.29-2.15 (br m, 5H), 2.06-1.93 (m, 1H), 1.97-1.79 (br m, 2H), 1.53-1.41 (br m, 2H), 0.95 (d, J=6.7 Hz, 3H).

### Example 53C 2″-{(2R)-3-[(4-methoxyphenyl)methoxy]-2-methylpropyl}-5″-methyldispiro[[1,3]dioxolane-2,1′-cyclohex[2]ene-4′,1″-isoindol]-3″(2″H)-one

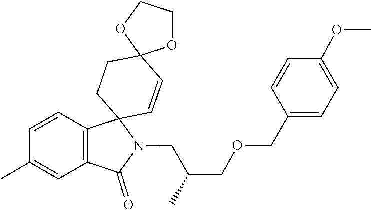

To a solution of Example 53B (15.8 g, 29.1 mmol, 1 eq) in MeCN (250 mL) was added PPh₃ (3.82 g, 14.6 mmol, 0.5 eq), TBACl (12.1 g, 43.6 mmol, 1.5 eq) and K₂CO₃ (12.1 g, 87.3 mmol, 3 eq). The mixture was sparged with N₂ (10 min) then Pd(OAc)₂ (980 mg, 4.36 mmol, 0.15 eq) was added and the mixture was heated in a sealed flask at 120° C. for 24 h. The mixture was allowed to cool to rt, filtered through celite (10 g column) and the filtrate was partitioned between EtOAc and water, and the organic phase was washed with brine, dried (MgSO₄) and concentrated in vacuo. Purification by automated flash chromatography (CombiFlash Rf, 220 g RediSep™ silica cartridge) eluting with a gradient of 0-50% EtOAc in heptane afforded a mixture of diastereoisomers, Example 53C as a yellow gum (10.1 g, 21.9 mmol, 75%). LRMS calculated for C₂₈H₃₃NO₅: 463; found: 464 (M+H). ¹H NMR (400 MHz, DMSO-d₆) δ ppm: 7.50 (t, J=1.9 Hz, 1H), 7.45-7.40 (m, 1H), 7.37-7.33 (m, 1H), 7.27-7.19 (m, 2H), 6.92-6.84 (m, 2H), 5.99-5.92 (m, 1H), 5.42-5.32 (m, 1H), 4.43-4.31 (m, 2H), 4.07-3.87 (m, 4H), 3.74/3.73 (s, 3H), 3.44-3.10 (m, 4H), 2.40 (s, 3H), 2.38-2.26 (m, 2H), 2.25-2.14 (m, 1H), 2.05-1.96 (m, 1H), 1.81-1.72 (m, 1H), 0.90/0.85 (d, J=6.8 Hz, 3H).

### Example 53D 2″-{(2R)-3-[(4-methoxyphenyl)methoxy]-2-methylpropyl}-5″-methyldispiro[[1,3]dioxolane-2,1′-cyclohexane-4′,1″-isoindol]-3″(2″H)-one

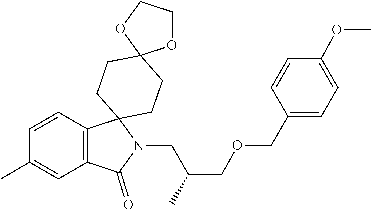

To a solution of Example 53C (14.9 g, 32.2 mmol, 1 eq) and HCOONH₄ (40.6 g, 645 mmol, 20 eq) in EtOH (700 mL) was added a catalytic amount of Pd/C (1.03 g, 9.67 mmol, 0.3 eq).

The mixture was stirred for 22 h at rt. The reaction was filtered through celite, eluted with MeOH and evaporated under reduced pressure. The residue was partitioned between EtOAc and water, and the organic phase was washed with brine, dried (MgSO₄) and concentrated in vacuo to afford Example 53D as a colourless gum (12.1 g, 26 mmol, 81%). LRMS calculated for C₂₈H₃₅NO₅: 465; found: 466 (M+H). ¹H NMR (400 MHz, DMSO-d₆) δ ppm: 7.73 (d, J=7.9 Hz, 1H), 7.53-7.50 (m, 1H), 7.44-7.39 (m, 1H), 7.28-7.22 (m, 2H), 6.92-6.87 (m, 2H), 4.40-4.33 (m, 2H), 4.00-3.91 (m, 4H), 3.74 (s, 3H), 3.37-3.27 (m, 3H), 3.18 (dd, J=14.0, 7.7 Hz, 1H), 2.49-2.42 (m, 1H), 2.40 (s, 3H), 2.34-2.17 (m, 2H), 2.17-2.05 (m, 2H), 1.88-1.78 (m, 2H), 1.39-1.25 (m, 2H), 0.90 (d, J=6.8 Hz, 3H).

### Example 53E 2″-{(2R)-3-[(4-methoxyphenyl)methoxy]-2-methylpropyl}-5″-methyl-2″,3″-dihydrodispiro[[1,3]dioxolane-2,1′-cyclohexane-4′,1″-isoindole]

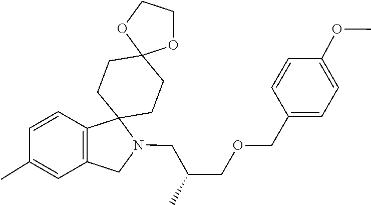

To a solution of Example 53D (12.1 g, 26 mmol, 1 eq) in THE (200 mL), cooled to 0° C. was added LAH (39 mL, 1 M, 39 mmol, 1.5 eq) and the mixture was stirred at 0° C. for 15 min, then at rt for 2 h. The mixture was cooled to 0° C., water (1.46 mL) was added followed by 15% aq. NaOH solution (1.46 mL) then water (4.4 mL) and the mixture was stirred for 30 min. The mixture was filtered through celite, washed with EtOAc and concentrated in vacuo. Purification by automated flash chromatography (CombiFlash Rf, 120 g RediSep™ silica cartridge) eluting with a gradient of 0-40% EtOAc in heptane afforded Example 53E as a pink gum (10 g, 22.2 mmol, 86%). LRMS calculated for C₂₈H₃₇NO₄: 451; found: 452 (M+H). ¹H NMR (400 MHz, DMSO-d₆) δ ppm: 7.37 (d, J=7.8 Hz, 1H), 7.27-7.22 (m, 2H), 7.08-7.04 (m, 1H), 7.01 (dd, J=7.8, 1.7 Hz, 1H), 6.92-6.87 (m, 2H), 4.40-4.32 (m, 2H), 3.96-3.86 (m, 4H), 3.86-3.76 (m, 2H), 3.74 (s, 3H), 3.38 (dd, J=9.0, 4.8 Hz, 1H), 3.31 (dd, J=9.0, 5.8 Hz, 1H), 2.59 (dd, J=11.9, 7.3 Hz, 1H), 2.33 (dd, J=11.9, 7.0 Hz, 1H), 2.28 (s, 3H), 1.97-1.79 (m, 5H), 1.76-1.68 (m, 2H), 1.45-1.37 (m, 2H), 0.91 (d, J=6.7 Hz, 3H).

### Example 53F 2′-[(2R)-3-hydroxy-2-methylpropyl]-5′-methyl-2′,3′-dihydrospiro[cyclohexane-1,1′-isoindol]-4-one

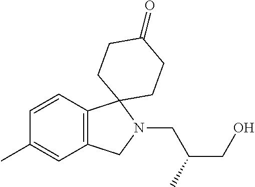 [SMILES: Cc1ccc2c(c1)CN(C[C@@H](C)CO)C21CCC(=O)CC1]

To a solution of Example 53E (9.5 g, 21.0 mmol, 1.0 eq) in THF/water (1:1, 150 mL) was added PTSA (20.8 g, 109 mmol, 5.2 eq) and the mixture was heated at 85° C. for 18 h. The mixture was allowed to cool to rt and partitioned between EtOAc and sat. aq. NaHCO₃ solution, and the combined organic extracts were washed with brine, dried (MgSO₄) and concentrated in vacuo. Purification by automated flash column chromatography (CombiFlash Rf, 330 g Gold RediSep™ silica cartridge) eluting with a gradient of 0-60% EtOAc in heptane afforded Example 53F as an oil (4.9 g, 17.1 mmol, 81%). ¹H NMR (400 MHz, DMSO-d₆) δ ppm: 7.45 (d, J=7.8 Hz, 1H), 7.12-7.08 (m, 1H), 7.06-7.00 (m, 1H), 4.53 (dd, J=5.8, 4.6 Hz, 1H), 3.90 (d, J=13.1 Hz, 1H), 3.84 (d, J=13.1 Hz, 1H), 3.45-3.37 (m, 1H), 3.33-3.27 (m, 1H), 2.72-2.59 (m, 3H), 2.46-2.32 (m, 3H), 2.30 (s, 3H), 2.17-2.04 (m, 2H), 1.84-1.71 (m, 3H), 0.86 (d, J=6.7 Hz, 3H).

### Example 53G (1'S,1″s)-2″-[(2R)-3-hydroxy-2-methylpropyl]-5″-methyl-2″,3″-dihydrodispiro[imidazolidine-4,1′-cyclohexane-4′,1″-isoindole]-2,5-dione

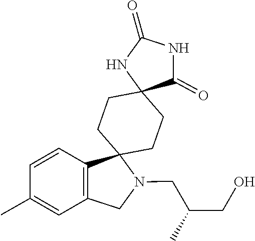 [SMILES: Cc1ccc2c(c1)CN(C[C@@H](C)CO)[C@]21CC[C@]2(CC1)NC(=O)NC2=O]

To a solution of Example 53F (4.8 g, 16.7 mmol, 1.0 eq) in EtOH (110 mL) and water (110 mL) was added NaCN (1.64 g, 33.4 mmol, 2.0 eq) and (NH₄)₂CO₃ (6.42 g, 66.8 mmol, 4.0 eq). The mixture was heated at 60° C. for 18 h. A mixture of diastereoisomers was obtained. The mixture was cooled to rt and the precipitate was collected by filtration, washed with ice cold water and dried under vacuum to afford a single diastereoisomer Example 53G as a white solid (3.8 g, 10.6 mmol, 64%). LRMS calculated for C₂₀H₂₇N₃O₃: 357; found: 358 (M+H). ¹H NMR (400 MHz, DMSO-d₆) δ ppm: 10.71 (s, 1H), 8.87 (s, 1H), 7.40 (d, J=7.8 Hz, 1H), 7.11-7.08 (m, 1H), 7.08-7.03 (m, 1H), 4.61 (t, J=5.0 Hz, 1H), 3.93 (d, J=13.0 Hz, 1H), 3.83 (d, J=13.0 Hz, 1H), 3.46 (ddd, J=9.4, 5.6, 3.2 Hz, 1H), 3.30-3.22 (m, 1H), 2.81 (dd, J=12.3, 7.6 Hz, 1H), 2.59-2.52 (m, 1H), 2.29 (s, 3H), 2.27-2.10 (m, 2H), 1.95-1.73 (m, 3H), 1.67-1.56 (m, 2H), 1.49-1.37 (m, 2H), 0.87 (d, J=6.6 Hz, 3H).

### Example 53H (1s,4S)-4-amino-2′-[(2R)-3-hydroxy-2-methylpropyl]-5′-methyl-2′,3′-dihydrospiro[cyclohexane-1,1′-isoindole]-4-carboxylic acid

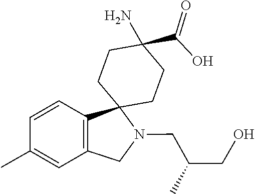 [SMILES: Cc1ccc2c(c1)CN(C[C@@H](C)CO)[C@]21CC[C@](N)(C(=O)O)CC1]

To a solution of Example 53G (3.80 g, 10.6 mmol, 1.0 eq) in water (50 mL) was added LiOH×H₂O (4.46 g, 106 mmol, 10.0 eq) and the mixture was heated in a sealed flask at 150° C. for 18 h. The reaction was allowed to cool to 0° C., adjusted to pH 7 with 2 M aq. HCl solution and the solids were collected by filtration, washed with water and dried under vacuum to afford Example 53H as a white solid (4.29 g, overweight). LRMS calculated for C₁₉H₂₈N₂O₃: 332; found: 333 (M+H). ¹H NMR (400 MHz, DMSO-d₆) δ ppm: 7.70 (br s, 2H), 7.63 (d, J=7.9 Hz, 1H), 7.09-7.05 (m, 1H), 7.05-6.99 (m, 1H), 4.64 (br s, 1H), 3.92 (d, J=12.9 Hz, 1H), 3.82 (d, J=12.9 Hz, 1H), 3.45 (dd, J=10.3, 5.9 Hz, 1H), 3.27 (dd, J=10.3, 6.2 Hz, 1H), 2.79 (dd, J=12.2, 7.6 Hz, 1H), 2.56-2.40 (m, 3H), 2.29 (s, 3H), 1.87-1.71 (m, 3H), 1.71-1.60 (m, 2H), 1.39-1.27 (m, 2H), 0.86 (d, J=6.6 Hz, 3H).

### Example 531 (1s,4S)-4-(3-chloro-2-methylanilino)-2′-[(2R)-3-hydroxy-2-methylpropyl]-5′-methyl-2′,3′-dihydrospiro[cyclohexane-1,1′-isoindole]-4-carboxylic acid

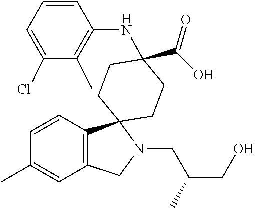 [SMILES: Cc1ccc2c(c1)CN(C[C@@H](C)CO)[C@]21CC[C@](Nc2cccc(Cl)c2C)(C(=O)O)CC1]

To a solution of Example 53H (1.23 g, 3.06 mmol, 0.83 eq) in DMF (25 mL) was added Cs₂CO₃ (2.4 g, 7.37 mmol, 2 eq), ethyl 2-cyclohexanonecarboxylate (0.24 mL, 1.47 mmol, 0.4 eq) and 1-chloro-3-iodo-2-methylbenzene (0.57 mL, 4.05 mmol, 1.1 eq). The mixture was sparged with N₂ (10 min) before the addition of CuI (70 mg, 0.37 mmol, 0.1 eq) and the mixture was heated in a sealed flask at 150° C. for 2.5 h. The mixture was cooled to rt, MeOH was added and the solids were collected by filtration. The filtrate was concentrated in vacuo and purification by automated flash column chromatography (CombiFlash Rf, 40 g Gold RediSep™ silica cartridge) eluting with a gradient of 0-10% MeOH in DCM afforded Example 531 as a brown gum (801 mg, 1.75 mmol, 57%). LRMS calculated for C₂₆H₃₃ClN₂O₃: 456; found: 457 (M+H). ¹H NMR (400 MHz, DMSO-d₆) δ ppm: 7.43 (d, J=7.8 Hz, 1H), 7.09-7.07 (m, 1H), 7.06-7.02 (m, 1H), 6.94 (t, J=8.1 Hz, 1H), 6.70 (dd, J=8.0, 1.0 Hz, 1H), 6.36 (dd, J=8.3, 1.1 Hz, 1H), 4.96 (br s, 1H), 3.87 (d, J=12.9 Hz, 1H), 3.81 (d, J=12.9 Hz, 1H), 3.40 (dd, J=10.3, 5.8 Hz, 1H), 3.26 (dd, J=10.3, 5.7 Hz, 1H), 2.62 (dd, J=11.9, 7.4 Hz, 1H), 2.42 (dd, J=11.9, 7.0 Hz, 1H), 2.32 (s, 3H), 2.29 (s, 3H), 2.26-2.10 (m, 4H), 1.96-1.83 (m, 2H), 1.83-1.72 (m, 1H), 1.29-1.17 (m, 2H), 0.84 (d, J=6.7 Hz, 3H).

### Example 53J methyl (1s,4S)-4-(3-chloro-2-methylanilino)-2′-[(2R)-3-hydroxy-2-methylpropyl]-5′-methyl-2′,3′-dihydrospiro[cyclohexane-1,1′-isoindole]-4-carboxylate

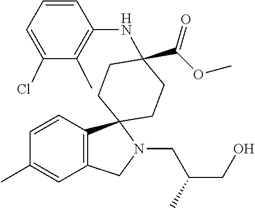 [SMILES: COC(=O)[C@]1(Nc2cccc(Cl)c2C)CC[C@@]2(CC1)c1ccc(C)cc1CN2C[C@@H](C)CO]

To a solution of Example 531 (853 mg, 1.87 mmol, 1 eq) in DCM (10 mL) and MeOH (10 mL), cooled to 0° C., was added TMS-CHNN (9.33 mL, 0.6 M, 5.6 mmol, 3 eq) dropwise and the mixture was stirred for 1.5 h. AcOH (0.5 mL) was added dropwise and the mixture was concentrated in vacuo. The residue was partitioned between EtOAc and sat. aq. NaHCO₃ solution, and the organic phase was washed with brine, dried (MgSO₄) and concentrated in vacuo. Purification by automated flash chromatography (CombiFlash Rf, 40 g Gold RediSep™ silica cartridge) eluting with a gradient of 0-54% EtOAc in heptane afforded Example 53J as a white foam (573 mg, 1.22 mmol, 65%). LRMS calculated for C₂₇H₃₅ClN₂O₃: 470; found: 471 (M+H). ¹H NMR (400 MHz, DMSO-d₆) δ ppm: 7.42 (d, J=7.8 Hz, 1H), 7.10-7.07 (m, 1H), 7.07-7.02 (m, 1H), 6.97 (t, J=8.1 Hz, 1H), 6.75 (dd, J=8.0, 1.0 Hz, 1H), 6.18 (dd, J=8.2, 1.1 Hz, 1H), 5.21 (s, 1H), 4.58 (dd, J=5.9, 4.2 Hz, 1H), 3.90-3.79 (m, 2H), 3.69 (s, 3H), 3.40 (ddd, J=10.0, 5.6, 4.1 Hz, 1H), 3.26 (dt, J=10.2, 5.8 Hz, 1H), 2.62 (dd, J=11.9, 7.2 Hz, 1H), 2.42 (dd, J=11.9, 7.2 Hz, 1H), 2.35 (s, 3H), 2.29 (s, 3H), 2.28-2.11 (m, 4H), 1.98-1.85 (m, 2H), 1.83-1.71 (m, 1H), 1.30-1.21 (m, 2H), 0.84 (d, J=6.6 Hz, 3H).

### Example 53K methyl (1s,4S)-4-(3-chloro-2-methylanilino)-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

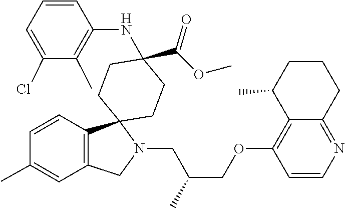 [SMILES: COC(=O)[C@]1(Nc2cccc(Cl)c2C)CC[C@@]2(CC1)c1ccc(C)cc1CN2C[C@@H](C)COc1ccnc2c1[C@H](C)CCC2]

To a solution of Example 53J (573 mg, 1.22 mmol, 1 eq) in THE (15 mL) was added Preparation 2a1 (397 mg, 2.43 mmol, 2 eq) and PPh₃ (638 mg, 2.43 mmol, 2 eq), followed by DIAD (560 mg, 2.43 mmol, 2 eq). The mixture was stirred at 50° C. for 2 h. The mixture was partitioned between EtOAc and sat. aq. NaHCO₃ solution, and the organic phase was washed with brine, dried (MgSO₄) and concentrated in vacuo. The residue was dissolved in MeOH, then loaded onto a MeOH-wet SCX cartridge (10 g), washed successively with DCM, MeOH and eluted with 7 M NH₃ solution in MeOH/MeOH (1:5) and concentrated in vacuo. Purification by automated flash chromatography (CombiFlash Rf, 24 g Gold RediSep™ silica cartridge) eluting with a gradient of 0-100% EtOAc in heptane afforded Example 53K as a white foam (432 mg, 0.7 mmol, 58%). LRMS calculated for C₃₇H₄₆ClN₃O₃: 615; found: 616 (M+H). ¹H NMR (400 MHz, DMSO-d₆) δ ppm: 8.09 (d, J=5.6 Hz, 1H), 7.39 (d, J=7.8 Hz, 1H), 7.10-7.07 (m, 1H), 7.06-7.01 (m, 1H), 6.98 (t, J=8.1 Hz, 1H), 6.76 (dd, J=8.1, 1.0 Hz, 1H), 6.71 (d, J=5.6 Hz, 1H), 6.16 (dd, J=8.2, 1.1 Hz, 1H), 5.17 (s, 1H), 4.03 (dd, J=9.1, 4.3 Hz, 1H), 3.96 (d, J=12.8 Hz, 1H), 3.90 (dd, J=9.1, 3.6 Hz, 1H), 3.79 (d, J=12.8 Hz, 1H), 3.67 (s, 3H), 2.97-2.87 (m, 1H), 2.84-2.66 (m, 2H), 2.65-2.52 (m, 2H), 2.35 (s, 3H), 2.29 (s, 3H), 2.28-2.10 (m, 4H), 2.09-2.02 (m, 2H), 1.88-1.68 (m, 2H), 1.67-1.57 (m, 1H), 1.48-1.35 (m, 2H), 1.30-1.22 (m, 1H), 1.16-1.10 (m, 1H), 1.09 (app d, J=6.8 Hz, 6H).

### Example 53 (1s,4S)-4-(3-chloro-2-methylanilino)-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

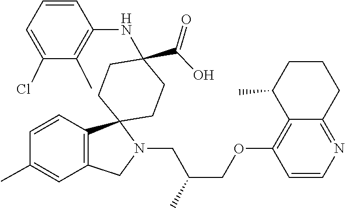 [SMILES: Cc1ccc2c(c1)CN(C[C@@H](C)COc1ccnc3c1[C@H](C)CCC3)[C@]21CC[C@](Nc2cccc(Cl)c2C)(C(=O)O)CC1]

To a solution of Example 53K (432 mg, 0.7 mmol, 1 eq) in 1,4 dioxane (10 mL) and water (2 mL) was added LiOH×H₂O (147 mg, 3.51 mmol, 5 eq) and the mixture was stirred at 80° C. for 6 h. The mixture was concentrated in vacuo. The residue was triturated in MeOH and the solids were collected by filtration, washed with MeOH and dried. Purification of the solid material by reverse phase automated flash chromatography at pH 4 (CombiFlash Rf, C18 30 g Gold RediSep column) eluting with a gradient of 10-100% MeCN in water afforded a solid. The solid was triturated in Et₂O, the solids were collected by filtration and dried under vacuum to afford Example 53 as a white solid (292 mg, 0.48 mmol, 69%). LRMS calculated for C₃₆H₄₄ClN₃O₃: 601; found: 602 (M+H). ¹H NMR (400 MHz, DMSO-d₆) δ ppm: 12.71 (br s, 1H), 8.09 (d, J=5.6 Hz, 1H), 7.40 (d, J=7.8 Hz, 1H), 7.10-7.07 (m, 1H), 7.06-7.01 (m, 1H), 6.98 (t, J=8.1 Hz, 1H), 6.77-6.69 (m, 2H), 6.30 (dd, J=8.2, 1.0 Hz, 1H), 4.99 (br s, 1H), 4.03 (dd, J=9.1, 4.1 Hz, 1H), 3.96 (d, J=12.8 Hz, 1H), 3.90 (dd, J=9.1, 3.6 Hz, 1H), 3.79 (d, J=12.8 Hz, 1H), 2.98-2.88 (m, 1H), 2.84-2.66 (m, 2H), 2.65-2.52 (m, 2H), 2.34 (s, 3H), 2.29 (s, 3H), 2.27-1.98 (m, 6H), 1.87-1.68 (m, 2H), 1.68-1.58 (m, 1H), 1.47-1.34 (m, 2H), 1.29-1.23 (m, 1H), 1.16-1.09 (m, 1H), 1.09 (app d, J=6.9 Hz, 6H).

### Example 54

### Example 54A (1s,4S)-4-(3-chloro-4-fluoroanilino)-2′-[(2R)-3-hydroxy-2-methylpropyl]-5′-methyl-2′,3′-dihydrospiro[cyclohexane-1,1′-isoindole]-4-carboxylic acid

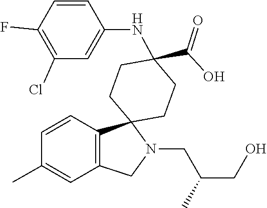 [SMILES: Cc1ccc2c(c1)CN(C[C@@H](C)CO)[C@]21CC[C@](Nc2ccc(F)c(Cl)c2)(C(=O)O)CC1]

To a solution of Example 53H (1.2 g, 2.98 mmol, 1.0 eq) in DMF (24 mL) was added Cs₂CO₃ (1.94 g, 5.95 mmol, 2.0 eq), ethyl 2-cyclohexanonecarboxylate (0.20 g, 0.19 mL, 1.19 mmol, 0.4 eq), 3-chloro-4-fluoroiodobenzene, (0.84 g, 3.28 mmol, 1.1 eq) and the mixture was sparged with N₂ (10 min). CuI (60 mg, 0.30 mmol, 0.1 eq) was added and then the flask sealed and heated at 150° C. for 2.5 h. The mixture was cooled to rt, diluted with MeOH and the solids were collected by filtration. The filtrate was concentrated in vacuo and purification by automated flash column chromatography (CombiFlash Rf, 40 g Gold RediSep™ silica cartridge) eluting with a gradient of 0-10% MeOH in DCM afforded Example 54A as an orange solid (667 mg, 1.45 mmol, 49%). LRMS calculated for C₂₅H₃₀ClFN₂O₃: 460; found 461 (M+H).

### Example 54B methyl (1s,4S)-4-(3-chloro-4-fluoroanilino)-2′-[(2R)-3-hydroxy-2-methylpropyl]-5′-methyl-2′,3′-dihydrospiro[cyclohexane-1,1′-isoindole]-4-carboxylate

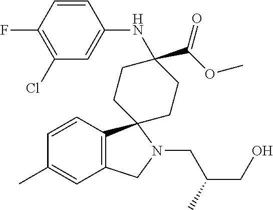 [SMILES: COC(=O)[C@]1(Nc2ccc(F)c(Cl)c2)CC[C@@]2(CC1)c1ccc(C)cc1CN2C[C@@H](C)CO]

To a solution of Example 54A (667 mg, 1.45 mmol, 1.0 eq) in DCM (9 mL) and MeOH (9 mL) at 0° C., was added TMS-CHNN (7.25 mL, 0.6 M, 4.35 mmol, 3 eq) dropwise and the mixture was stirred for a further 1 h at 0° C. AcOH (0.5 mL) was added dropwise and after stirring for 30 min the mixture was concentrated in vacuo. The residue was partitioned between EtOAc and sat. aq. NaHCO₃ solution, and the organic phase was washed with brine, dried (MgSO₄) and concentrated in vacuo. Purification by automated flash chromatography (CombiFlash Rf, 24 g Gold RediSep™ silica cartridge) eluting with a gradient of 0-50% EtOAc in heptane afforded Example 54B as a white foam (492 mg, 1.04 mmol, 72%). LRMS calculated for C₂₅H₃₀ClFN₂O₃: 474; found 475 (M+H).

### Example 54 (1s,4S)-4-(3-chloro-4-fluoroanilino)-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

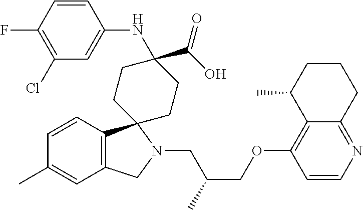 [SMILES: Cc1ccc2c(c1)CN(C[C@@H](C)COc1ccnc3c1[C@H](C)CCC3)[C@]21CC[C@](Nc2ccc(F)c(Cl)c2)(C(=O)O)CC1]

Using General procedure 32 and Example 54B as the appropriate isoindoline (492 mg, 1.04 mmol, 1 eq) and Preparation 2a1 (339 mg, 2.08 mmol, 2 eq) as the appropriate alcohol, Example 54 was obtained as a white solid (315 mg, 0.52 mmol, 77%). HRMS calculated for C₃₅H₄₁N₃O₃FCl: 605.2820; found: 606.2895 (M+H). ¹H NMR (400 MHz, DMSO-d₆) δ ppm: 8.11 (d, J=5.6 Hz, 1H), 7.38 (d, J=7.8 Hz, 1H), 7.16-7.06 (m, 2H), 7.03 (d, J=7.9 Hz, 1H), 6.73 (d, J=5.6 Hz, 1H), 6.67 (dd, J=6.3, 2.8 Hz, 1H), 6.51 (ddd, J=9.0, 3.8, 2.8 Hz, 1H), 6.12 (bs, 1H), 4.02-3.88 (m, 3H), 3.84 (d, J=12.9 Hz, 1H), 3.05-2.96 (m, 1H), 2.82-2.68 (m, 2H), 2.67-2.52 (m, 2H), 2.29 (s, 3H), 2.28-1.97 (m, 6H), 1.91-1.71 (m, 2H), 1.69-1.59 (m, 1H), 1.54-1.40 (m, 2H), 1.36-1.27 (m, 1H), 1.22-1.14 (m, 1H), 1.11 (d, J=6.8 Hz, 3H), 1.08 (d, J=6.7 Hz, 3H).

### Example 55

### Example 55A (1s,4S)-4-(3-chloroanilino)-2′-[(2R)-3-hydroxy-2-methylpropyl]-5′-methyl-2′,3′-dihydrospiro[cyclohexane-1,1′-isoindole]-4-carboxylic acid

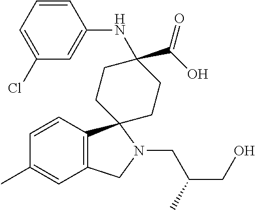 [SMILES: Cc1ccc2c(c1)CN(C[C@@H](C)CO)[C@]21CC[C@](Nc2cccc(Cl)c2)(C(=O)O)CC1]

To a solution of Example 53H (1.0 g, 2.98 mmol, 1.0 eq) in DMF (20 mL) was added Cs₂CO₃ (1.62 g, 4.96 mmol, 2.0 eq), ethyl 2-cyclohexanonecarboxylate (0.16 mL, 0.99 mmol, 0.4 eq), 3-chloroiodobenzene, (0.34 mL, 2.73 mmol, 1.1 eq) and the mixture was sparged with N₂ (10 min). CuI (50 mg, 0.25 mmol, 0.1 eq) was added and then the flask sealed and heated at 150° C. for 2.5 h. The mixture was cooled to rt, diluted with MeOH and the solids were collected by filtration. The filtrate was concentrated in vacuo and purification by automated flash column chromatography (CombiFlash Rf, 40 g Gold RediSep™ silica cartridge) eluting with a gradient of 0-10% MeOH in DCM afforded Example 55A as an orange solid (580 mg, 1.31 mmol, 53%). LRMS calculated for C₂₅H₃₁ClN₂O₃: 442; found 443 (M+H).

### Example 55B methyl (1s,4S)-4-(3-chloroanilino)-2′-[(2R)-3-hydroxy-2-methylpropyl]-5′-methyl-2′,3′-dihydrospiro[cyclohexane-1,1′-isoindole]-4-carboxylate

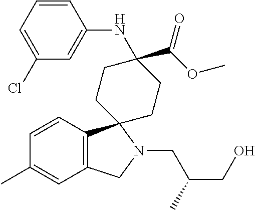 [SMILES: COC(=O)[C@]1(Nc2cccc(Cl)c2)CC[C@@]2(CC1)c1ccc(C)cc1CN2C[C@@H](C)CO]

To a solution of Example 55A (580 mg, 1.31 mmol, 1.0 eq) in DCM (8 mL) and MeOH (8 mL) at 0° C., was added TMS-CHNN (6.55 mL, 0.6 M, 3.93 mmol, 3 eq) dropwise and the mixture was stirred for a further 1 h at 0° C. AcOH (0.4 mL) was added dropwise and after stirring 30 min the mixture was concentrated in vacuo. The residue was partitioned between EtOAc and sat. aq. NaHCO₃ solution, and the organic phase was washed with brine, dried (MgSO₄) and concentrated in vacuo. Purification by automated flash chromatography (CombiFlash Rf, 24 g Gold RediSep™ silica cartridge) eluting with a gradient of 0-60% EtOAc in heptane afforded Example 55B as a white foam (402 mg, 0.88 mmol, 67%). LRMS calculated for C₂₆H₃₃ClN₂O₃: 456; found 457 (M+H).

### Example 55 (1s,4S)-4-(3-chloroanilino)-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

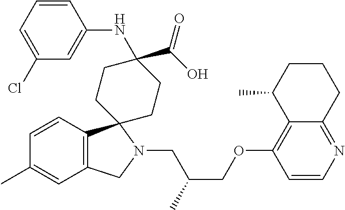 [SMILES: Cc1ccc2c(c1)CN(C[C@@H](C)COc1ccnc3c1[C@H](C)CCC3)[C@]21CC[C@](Nc2cccc(Cl)c2)(C(=O)O)CC1]

Using General procedure 32 and Example 55B as the appropriate isoindoline (200 mg, 0.44 mmol, 1 eq) and Preparation 2a1 (144 mg, 0.88 mmol, 2 eq) as the appropriate alcohol, Example 55 was obtained as a white solid (66 mg, 0.11 mmol, 38%). HRMS calculated for C₃₅H₄₂N₃O₃Cl: 587.2914; found: 588.2994 (M+H). ¹H NMR (400 MHz, DMSO-d₆) δ ppm: 12.56 (br s, 1H), 8.13 (d, J=5.7 Hz, 1H), 7.39 (d, J=7.9 Hz, 1H), 7.11-7.00 (m, 3H), 6.76 (d, J=5.7 Hz, 1H), 6.61 (t, J=2.1 Hz, 1H), 6.57 (ddd, J=7.8, 2.0, 0.8 Hz, 1H), 6.52 (ddd, J=8.3, 2.3, 0.9 Hz, 1H), 6.24 (br s, 1H), 4.02 (dd, J=9.4, 3.9 Hz, 1H), 3.99-3.89 (m, 2H), 3.86 (d, J=12.9 Hz, 1H), 3.08-2.98 (m, 1H), 2.81-2.69 (m, 2H), 2.68-2.52 (m, 2H), 2.29 (s, 3H), 2.29-1.97 (m, 6H), 1.93-1.71 (m, 2H), 1.71-1.60 (m, 1H), 1.56-1.42 (m, 2H), 1.37-1.27 (m, 1H), 1.24-1.15 (m, 1H), 1.12 (d, J=6.8 Hz, 3H), 1.08 (d, J=6.7 Hz, 3H).

### Example 56

### Example 56A 2-bromo-5-chlorobenzoyl chloride

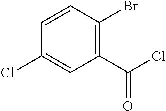 [SMILES: O=C(Cl)c1cc(Cl)ccc1Br]

To a solution of 2-bromo-5-chlorobenzoic acid, (42 g, 178.37 mmol, 1 eq) in DCM (750 mL) was added oxalyl chloride, 2.OM in DCM (178.37 mL, 356.75 mmol, 2 eq) dropwise and cooled to 0° C. DMF (1 mL) was added dropwise and after addition was stirred for a further 10 min at 0° C. and rt for 2 h. The mixture was concentrated in vacuo and to the residue was added anhydrous DCM (100 mL) and this was also removed in vacuo (repeated twice) to give Example 56A as a brown solid (45.9 g, 178.37 mmol, 100%) that was used without further purification.

### Example 56B 2-bromo-5-chloro-N-(1,4-dioxaspiro[4.5]dec-7-en-8-yl)-N-{(2R)-3-[(4-methoxyphenyl)methoxy]-2-methylpropyl}benzamide

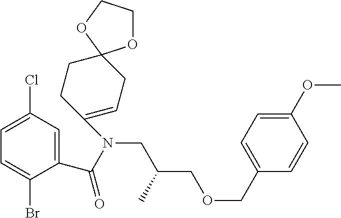 [SMILES: COc1ccc(COC[C@H](C)CN(C(=O)c2cc(Cl)ccc2Br)C2=CCC3(CC2)OCCO3)cc1]

A solution of 1,4-dioxaspiro[4.5]decan-8-one (17.07 g, 109.32 mmol, 1 eq) and Preparation 3e (28.6 g, 109.32 mmol, 1.0 eq) in CHCl₃ (100 mL) was stirred for 18 h with 4\acute{Å} molecular sieves (6 g). The solids were collected by filtration, washed with DCM and the filtrate was concentrated in vacuo. To a solution of the residue in DCM (100 mL) was added TEA (34.82 mL, 250.27 mmol, 2.3 eq), cooled to 0° C., followed by the dropwise addition of Example 56A (33.49 g, 0.13 mol, 1.2 eq) in DCM (150 mL). After complete addition the mixture was stirred at rt for 18 h. The mixture was partitioned between DCM sat. aq. NaHCO₃ solution, and the organic phase was washed with brine, dried (MgSO₄) and concentrated in vacuo. Purification by automated flash chromatography (CombiFlash Rf, 330 g Gold RediSep™ silica cartridge) eluting with a gradient of 0-25% EtOAc in heptane afforded Example 56B as a brown oil (38.53 g, 68.21 mmol, 62%). LRMS calculated for C₂₇H₃₁BrClNO₅: 563; found 564 (M+H).

### Example 56C 5″-chloro-2″-{(2R)-3-[(4-methoxyphenyl)methoxy]-2-methylpropyl}dispiro[[1,3]dioxolane-2,1′-cyclohex[2]ene-4′,1″-isoindol]-3″(2″H)-one

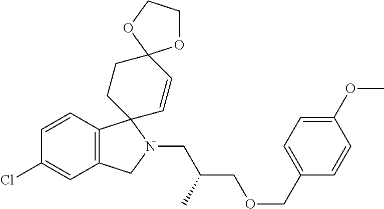 [SMILES: COc1ccc(COC[C@H](C)CN2Cc3cc(Cl)ccc3C23C=CC2(CC3)OCCO2)cc1]

Using General procedure 39 and Example 56B as the appropriate benzamide, a mixture of diastereoisomers, Example 56C was obtained as a brown oil. LRMS calculated for C₂₇H₃₀ClNO₅: 483; found 484 (M+H).

### Example 56D 5″-chloro-2″-{(2R)-3-[(4-methoxyphenyl)methoxy]-2-methylpropyl}-2″,3″-dihydrodispiro[[1,3]dioxolane-2,1′-cyclohex[2]ene-4′,1″-isoindole]

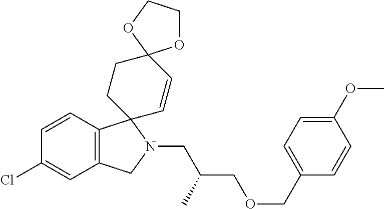 [SMILES: COc1ccc(COC[C@H](C)CN2Cc3cc(Cl)ccc3C23C=CC2(CC3)OCCO2)cc1]

Using General procedure 38 and Example 56C as the appropriate isoindolin-1-one, Example 56D was obtained as a mixture of diastereoisomers. LRMS calculated for C₂₇H₃₂ClNO₄: 469; found 470 (M+H).

### Example 56E 5′-chloro-2′-{(2R)-3-[(4-methoxyphenyl)methoxy]-2-methylpropyl}-2′,3′-dihydrospiro[cyclohex-2-ene-1,1′-isoindol]-4-one

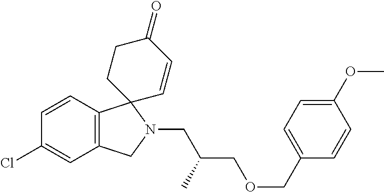 [SMILES: COc1ccc(COC[C@H](C)CN2Cc3cc(Cl)ccc3C23C=CC(=O)CC3)cc1]

To a solution of Example 56D (20.82 g, 44.30 mmol, 1.0 eq) in THE (160 mL) and water (160 mL) was added PTSA (25.28 g, 142.69 mmol, 3.22 eq) and the mixture was heated at 85° C. for 1 h. The mixture was cooled to rt and partitioned between EtOAc and sat. aq. NaHCO₃ solution, and the combined organic extracts were washed with brine, dried (MgSO₄) and concentrated in vacuo. Purification by automated flash column chromatography (CombiFlash Rf, 330 g Gold RediSep™ silica cartridge) eluting with a gradient of 0-25% EtOAc in heptane afforded a mixture of diastereoisomers, Example 56E as a colourless oil (7.06 g, 16.57 mmol, 37%). LRMS calculated for C₂₅H₂₈ClNO₃: 425; found 426 (M+H).

### Example 56F 5′-chloro-2′-{(2R)-3-[(4-methoxyphenyl)methoxy]-2-methylpropyl}-4-[(triethylsilyl)oxy]-2′,3′-dihydrospiro[cyclohex-3-ene-1,1′-isoindole]

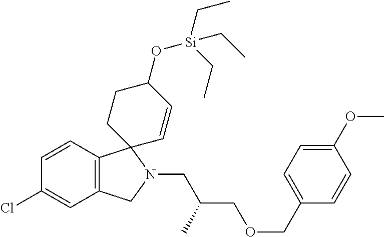 [SMILES: CC[Si](CC)(CC)OC1C=CC2(CC1)c1ccc(Cl)cc1CN2C[C@@H](C)COCc1ccc(OC)cc1]

To a solution of Example 56E (7.06 g, 16.57 mmol, 1 eq) in triethyl silane (24 mL, 150.26 mmol, 9.07 eq) was added Wilkinson's catalyst (383 mg, 0.41 mmol, 0.03 eq) and the mixture was heated at 60° C. for 2 h. Wilkinson's catalyst was added (80 mg, 0.065 mmol) and the mixture was heated at 60° C. for a further 15 min. The mixture was cooled to rt and concentrated in vacuo. The residue was purified by automated flash column chromatography (CombiFlash Rf, 220 g RediSep™ silica cartridge) eluting with a gradient of 0-30% EtOAc in heptane. Further purification by automated flash column chromatography (CombiFlash Rf, 120 g RediSep™ silica cartridge) eluting with a gradient of 0-25% EtOAc in heptane afforded a mixture of diastereoisomers, Example 56F as a colourless oil (6.4 g, 11.8 mmol, 71%). LRMS calculated for C₃₁H₄₄ClNO₃Si: 541; found 542 (M+H).

### Example 56G 5′-chloro-2′-{(2R)-3-[(4-methoxyphenyl)methoxy]-2-methylpropyl}-2′,3′-dihydrospiro[cyclohexane-1,1′-isoindol]-4-one

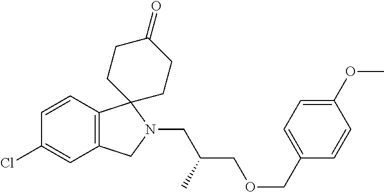 [SMILES: COc1ccc(COC[C@H](C)CN2Cc3cc(Cl)ccc3C23CCC(=O)CC3)cc1]

To a solution of Example 56F (6.38 g, 11.77 mmol, 1 eq) in THE (100 mL) at 0° C. was added TBAF dropwise (14.12 mL, 1 M in THF, 14.12 mmol, 1.2 eq). The mixture was stirred for a further 1 h at 0° C. and then partitioned between EtOAc and sat. NaHCO₃ solution, and the combined organic extracts were washed with brine, dried (MgSO₄) and concentrated in vacuo. The residue was purified by automated flash column chromatography (CombiFlash Rf, 120 g RediSep™ silica cartridge) eluting with a gradient of 0-30% EtOAc in heptane. Further purification by automated flash column chromatography (CombiFlash Rf, Gold 40 g RediSep™ silica cartridge) eluting with a gradient of 0-30% EtOAc in heptane afforded Example 56G as a colourless oil (2.84 g, 6.64 mmol, 56%). LRMS calculated for C₂₅H₃₀ClNO₃: 427; found 428 (M+H).

### Example 56H 5′-chloro-2′-[(2R)-3-hydroxy-2-methylpropyl]-2′,3′-dihydrospiro[cyclohexane-1,1′-isoindol]-4-one

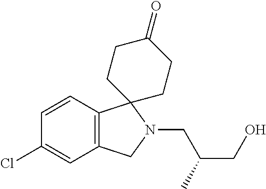 [SMILES: C[C@@H](CO)CN1Cc2cc(Cl)ccc2C12CCC(=O)CC2]

To a solution of Example 56G (2.84 g, 6.64 mmol, 1 eq) in DCM (60 mL) at 0° C. was added 1,3-dimethoxybenzene (2.61 mL, 19.91 mmol, 3 eq) followed by trifluoromethanesulfonic acid (0.58 mL, 6.64 mmol, 1 eq). The mixture was stirred for a further 1 h at 0° C. and trifluoromethanesulfonic acid (0.58 mL, 6.64 mmol, 1 eq) was added and stirring continued. NaHCO₃ solution was added and the mixture was extracted with DCM. The organic extract was washed with brine, dried (MgSO₄) and concentrated in vacuo. Purification by automated flash column chromatography (CombiFlash Rf, 120 g RediSep™ silica cartridge) eluting with a gradient of 0-52% EtOAc in heptane afforded Example 56H as an oil (960 mg, 3.12 mmol, 47%). LRMS calculated for C₁₇H₂₂ClNO₂: 307; found 308 (M+H).

### Example 56I (1'S,1″s)-5″-chloro-2″-[(2R)-3-hydroxy-2-methylpropyl]-2″,3″-dihydrodispiro[imidazolidine-4,1′-cyclohexane-4′,1″-isoindole]-2,5-dione

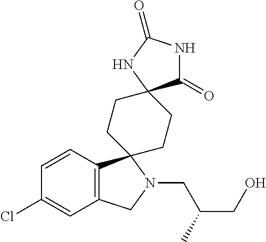 [SMILES: C[C@@H](CO)CN1Cc2cc(Cl)ccc2[C@]12CC[C@@]1(CC2)NC(=O)NC1=O]

To a suspension of Example 56H (960 mg, 3.12 mmol, 1 eq) in EtOH (13 mL) was added (NH₄)₂CO₃ (1.2 g, 12.48 mmol, 4 eq) and NaCN (306 mg, 6.24 mmol, 2 eq) and the mixture was heated at 60° C. for 18 h. The mixture was allowed to cool to rt and cold water and ice was added and it was stirred for 15 min. The solids were separated by filtration and taken up in DCM. The solvent was removed in vacuo and azeotroped twice with toluene. The resultant precipitate was triturated with Et₂O, the solids were separated by filtration, washed with further portions of cold Et₂O and dried in vacuo to give a single diastereoisomer, Example 561 as a white solid (650 mg 1.72 mmol, 55%). ¹H NMR (400 MHz, DMSO-d₆) δ ppm: 10.72 (s, 1H), 8.88 (s, 1H), 7.50 (d, J=8.2 Hz, 1H), 7.39 (d, J=2.1 Hz, 1H), 7.34 (dd, J=8.2, 2.1 Hz, 1H), 4.53 (br t, J=5.0 Hz, 1H), 3.94 (d, J=13.5 Hz, 1H), 3.88 (d, J=13.5 Hz, 1H), 3.49-3.40 (m, 1H), 3.30-3.21 (m, 1H), 2.79 (dd, J=12.4, 7.4 Hz, 1H), 2.59-2.51 (m, 1H), 2.14 (qd, J=14.2, 4.0 Hz, 2H), 1.88 (tt, J=14.1, 4.3 Hz, 2H), 1.81-1.70 (m, 1H), 1.69-1.59 (m, 2H), 1.51-1.42 (m, 2H), 0.88 (d, J=6.6 Hz, 3H).

### Example 56J (1s,4S)-4-amino-5′-chloro-2′-[(2R)-3-hydroxy-2-methylpropyl]-2′,3′-dihydrospiro[cyclohexane-1,1′-isoindole]-4-carboxylic acid

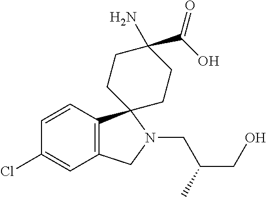 [SMILES: C[C@@H](CO)CN1Cc2cc(Cl)ccc2[C@]12CC[C@@](N)(C(=O)O)CC2]

To a suspension of Example 561 (680 mg, 1.80 mmol, 1 eq) in water (9 mL) was added LiOH×H₂O (755 mg, 18 mmol, 10 eq). The mixture was heated in a sealed tube at 150° C. for 18 h and then cooled to rt. Water was added, and the pH was adjusted to 7 by the addition of 1.2 M aq. HCl solution. The solids were removed by filtration, washed well with water and azeotroped twice with toluene to afford Example 56J as a beige solid (390 mg, 1.11 mmol, 61%). LRMS calculated for C₁₈H₂₅ClN₂O₃: 352; found 353 (M+H).

### Example 56K (1s,4S)-5′-chloro-4-(3-chloroanilino)-2′-[(2R)-3-hydroxy-2-methylpropyl]-2′,3′-dihydrospiro[cyclohexane-1,1′-isoindole]-4-carboxylic acid

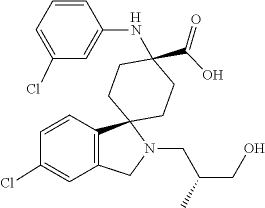

To a solution of Example 56J (390 mg, 1.11 mmol, 1 eq) in DMF (10 mL) was added 1-chloro-3-iodobenzene (0.14 mL, 1.11 mmol, 1 eq) and ethyl 2-cyclohexanonecarboxylate, (0.07 mL, 0.44 mmol, 0.4 eq). The mixture was degassed then Cs₂CO₃ (720 mg, 2.21 mmol, 2 eq) and CuI (21 mg, 0.11 mmol, 0.1 eq) were added. The mixture was degassed again and then heated in a sealed tube at 150° C. for 2.5 h. The reaction was cooled to rt and concentrated in vacuo.

Purification by flash chromatography (CombiFlash Rf, 40 g RediSep™ silica cartridge) eluting with a gradient of 0-10% MeOH in DCM afforded Example 56K as a brown solid (55 mg, 0.12 mmol, 11%). LRMS calculated for C₂₄H₂₈Cl₂N₂O₃: 462; found 463 (M+H).

### Example 56L methyl (1s,4S)-5′-chloro-4-(3-chloroanilino)-2′-[(2R)-3-hydroxy-2-methylpropyl]-2′,3′-dihydrospiro[cyclohexane-1,1′-isoindole]-4-carboxylate

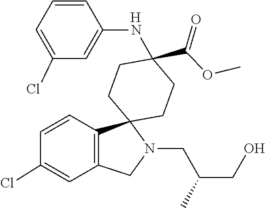

Using General procedure 17a and Example 56K as the appropriate amino acid (55 mg, 0.12 mmol, 1 eq), Example 56L was obtained as a beige foam (30 mg, 0.06 mmol, 53%). LRMS calculated for C₂₅H₃₀Cl₂N₂O₃: 476; found 477 (M+H).

### Example 56M methyl (1s,4S)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylate

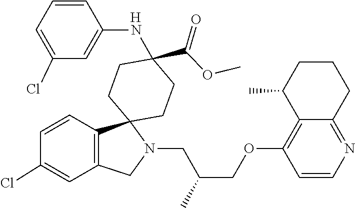

Using General procedure 30a and Example 56L as the appropriate isoindoline (28 mg, 0.06 mmol, 1 eq) and Preparation 2a1 (19 mg, 0.12 mmol, 2 eq) as the appropriate alcohol, Example 56M was obtained as a brown oil (18 mg, 0.03 mmol, 49%). LRMS calculated for C₃₅H₄₁Cl₂N₃O₃: 621; found 622 (M+H).

### Example 56 (1s,4S)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid

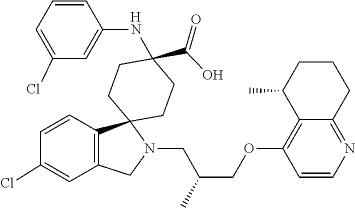

To a solution of Example 56M (70 mg, 0.11 mmol, 1 eq) in a mixture of 1,4-dioxane (0.8 mL) and water (0.2 mL) was added LiOH×H₂O (6 mg, 0.14 mmol, 5 eq) and the mixture was stirred at 90° C. for 10 h. The mixture was cooled to rt and concentrated in vacuo. Water was added and acidified to pH 5 using 1.2 M aq. HCl solution. The mixture was extracted with iPrOH:DCM (1:3) three times and the combined organic phase was washed with brine, dried (MgSO₄) and concentrated in vacuo. Purification by preparative HPLC automated flash chromatography (ISCO ACCQ, Prep HPLC Column: Gemini pH4 Dimensions: 21.2 mm×150 mm 5 μMSample) elution of A/B (95/5) to A/B (5/95), (A: water/0.1% HCOOH; B: MeCN/0.1% HCOOH) afforded Example 56 as an off-white solid (8.8 mg, 0.01 mmol, 50%). LRMS calculated for C₃₄H₃₉Cl₂N₃O₃: 607; found 608 (M+H). ¹H NMR (400 MHz, DMSO-d₆) δ ppm: δ 8.11 (d, J=5.6 Hz, 1H), 7.51 (d, J=8.2 Hz, 1H), 7.37 (d, J=2.1 Hz, 1H), 7.31 (dd, J=8.1, 2.1 Hz, 1H), 7.06 (t, J=8.1 Hz, 1H), 6.73 (d, J=5.6 Hz, 1H), 6.61 (t, J=2.1 Hz, 1H), 6.56 (dd, J=7.9, 1.7 Hz, 1H), 6.52 (dd, J=8.2, 1.8 Hz, 1H), 6.23 (br s, 1H), 4.03-3.87 (m, 4H), 3.07-2.98 (m, 1H), 2.80-2.69 (m, 2H), 2.66-2.52 (m, 2H), 2.26-1.98 (m, 6H), 1.94-1.61 (m, 3H), 1.57-1.43 (m, 2H), 1.39-1.30 (m, 1H), 1.27-1.18 (m, 1H), 1.11 (d, J=6.9 Hz, 3H), 1.08 (d, J=6.7 Hz, 3H).

### Example 57 4-(3-bromoanilino)-2′,3′-dihydrospiro[cyclohexane-1,1′-indene]-4-carboxylic acid

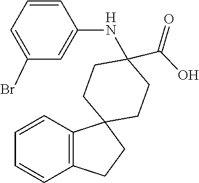

Using General procedure 10 and 2′,3′-dihydrospiro[cyclohexane-1,1′-indene]-4-one (100 mg, 0.5 mmol, 1.5 eq) as the appropriate ketone and 3-bromoaniline (36 μL, 0.33 mmol, 1 eq) as the appropriate aniline, a mixture of diastereoisomers was formed. After workup a single diastereoisomer, Example 57 was obtained as a white powder (6.5 mg, 0.02 mmol, 5%). LRMS calculated for C₂₁H₂₂NO₂Br: 399; found: 400 (M+H). ¹H NMR (400 MHz, DMSO-d₆) δ ppm: 7.23-7.07 (m, 4H), 7.01 (t, J=8.0 Hz, 1H), 6.83 (t, J=2.1 Hz, 1H), 6.69 (ddd, J=7.8, 1.9, 0.8 Hz, 1H), 6.63 (ddd, J=8.3, 2.3, 0.9 Hz, 1H), 2.85 (t, J=7.3 Hz, 2H), 2.45-2.37 (m, 2H), 1.98 (t, J=7.3 Hz, 2H), 1.84-1.51 (m, 6H).

### Example 58

### Example 58A dispiro[[1,3]dioxolane-2,1′-cyclohexane-4′,1″-indene]

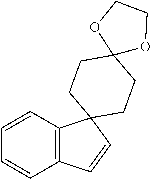 [SMILES: C1=CC2(CCC3(CC2)OCCO3)c2ccccc21]

NaH (60% dispersion; 362 mg, 9.04 mmol, 2.1 eq) was added to a solution of indene (500 mg, 4.3 mmol, 1 eq) and Preparation 1a (1.37 g, 4.3 mmol, 1 eq) in DMF (16 mL) cooled to 0° C., and then slowly allowed to warm to rt and stirred for 16 h. The reaction was quenched with sat. aq. NH₄Cl solution, stirred for 30 min and then concentrated in vacuo. The residue was diluted with sat. aq. NaHCO₃ solution and extracted with EtOAc. The combined organic extracts were washed with brine, dried (MgSO₄) and concentrated in vacuo. Purification by flash chromatography (25 g silica cartridge) eluting with a gradient of 0-10% EtOAc in heptane afforded Example 58A as a yellow solid (591 mg, 2.44 mmol, 57%). ¹H NMR (400 MHz, DMSO-d₆) δ ppm: 7.35-7.31 (m, 2H), 7.24-7.14 (m, 2H), 7.01 (d, J=5.6 Hz, 1H), 6.79 (d, J=5.7 Hz, 1H), 3.99-3.91 (m, 4H), 2.15-2.01 (m, 2H), 1.90-1.80 (m, 4H), 1.26-1.16 (m, 2H).

### Example 58B spiro[cyclohexane-1,1′-inden]-4-one

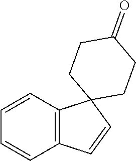 [SMILES: O=C1CCC2(C=Cc3ccccc32)CC1]

Using General procedure 9 and Example 58A (100 mg, 0.41 mmol, 1 eq) as the appropriate ketal in THE (3 mL), Example 58B was obtained as a yellow solid (80.9 mg, 0.41 mmol, 99%) that was used directly in the subsequent step without further purification. ¹H NMR (400 MHz, DMSO-d₆) δ ppm: 7.49-7.45 (m, 1H), 7.37-7.34 (m, 1H), 7.26-7.15 (m, 3H), 6.89 (d, J=5.6 Hz, 1H), 2.80-2.69 (m, 2H), 2.43-2.29 (m, 4H), 1.53-1.45 (m, 2H).

### Example 58 4-(3-bromoanilino)spiro[cyclohexane-1,1′-indene]-4-carboxylic acid, diastereoisomer 1

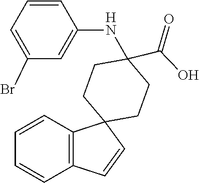

Using General procedure 10 and Example 58B (81 mg, 0.41 mmol, 1 eq) as the appropriate ketone and 3-bromoaniline (44 μL, 0.41 mmol, 1 eq) as the appropriate aniline, single diastereoisomer Example 58 was obtained as a cream foam (44.3 mg, 0.11 mmol, 27%). LRMS calculated for C₂₁H₂₀NO₂Br: 397; found: 398 (M+H). ¹H NMR (400 MHz, DMSO-d₆) δ ppm: 7.37-7.32 (m, 2H), 7.27-7.16 (m, 2H), 7.03 (t, J=8.0 Hz, 1H), 6.85 (t, J=2.1 Hz, 1H), 6.82-6.76 (m, 2H), 6.73-6.69 (m, 1H), 6.67-6.63 (m, 1H), 1.96-1.86 (m, 2H), 1.86-1.74 (m, 2H), 1.57-1.46 (m, 2H).

### Example 59

### Example 59A 7″-bromodispiro[[1,3]dioxolane-2,1′-cyclohexane-4′,1″-indene]

and

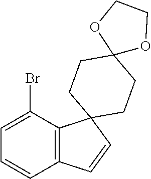 [SMILES: Brc1cccc2c1C1(C=C2)CCC2(CC1)OCCO2]

### Example 59B 4″-bromodispiro[[1,3]dioxolane-2,1′-cyclohexane-4′,1″-indene]

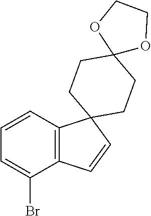 [SMILES: Brc1cccc2c1C=CC21CCC2(CC1)OCCO2]

To a solution of Preparation 1a (1.27 g, 4.00 mmol, 1.07 eq) and 4-bromoindene (0.73 g, 0.5 mL, 3.75 mmol. 1 eq) in DMF (16 mL), cooled to 0° C., under N₂, was added NaH (60% dispersion; 316 mg, 7.90 mmol, 2.1 eq) in one portion and the mixture allowed to warm to rt and stirred for 16 h. A mixture of regioisomers was obtained. The reaction was quenched with sat. aq. NH₄Cl solution and stirred for 30 min. The mixture was partitioned between EtOAc and sat. aq. NaHCO₃ solution. The organic phase was washed with brine, dried (MgSO₄), concentrated in vacuo and purified by automated flash chromatography (CombiFlash Rf, 40 g RediSep™ silica cartridge) eluting with a gradient of 0-10% EtOAc in heptane. The compound eluting earlier was collected as Example 59A (150 mg, 0.47 mmol, 13%). ¹H NMR (400 MHz, DMSO-d₆) δ ppm: 7.36-7.31 (m, 2H), 7.22-7.13 (m, 2H), 6.75 (d, J=5.7 Hz, 1H), 3.99-3.88 (m, 4H), 2.97 (td, J=13.1, 5.3 Hz, 2H), 1.91-1.75 (m, 4H), 1.07-0.99 (m, 2H).

The compound eluting later was collected as Example 59B (100 mg, 0.31 mmol, 8%). ¹H NMR (400 MHz, CD₃OD) δ ppm: 7.38-7.31 (m, 2H), 7.10 (t, J=7.7 Hz, 1H), 7.05 (d, J=5.7 Hz, 1H), 6.84 (d, J=5.7 Hz, 1H), 4.08-3.98 (m, 4H), 2.21 (td, J=12.6, 5.6 Hz, 2H), 1.98-1.84 (m, 4H), 1.40-1.30 (m, 2H).

### Example 59C 7′-bromospiro[cyclohexane-1,1′-inden]-4-one

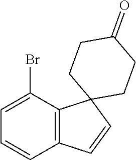 [SMILES: O=C1CCC2(C=Cc3cccc(Br)c32)CC1]

Using General procedure 9 and Example 59A (150 mg, 0.47 mmol, 1 eq) as the appropriate ketal in THF (3 mL), Example 59C was isolated as a yellow solid (130 mg, 0.47 mmol, 100%). ¹H NMR (400 MHz, DMSO-d₆) δ ppm: 7.46 (d, J=5.7 Hz, 1H), 7.40 (dd, J=7.4, 1.0 Hz, 1H), 7.38-7.34 (m, 1H), 7.20 (dd, J=8.0, 7.4 Hz, 1H), 6.88 (d, J=5.7 Hz, 1H), 3.04 (td, J=13.6, 4.5 Hz, 2H), 2.81 (td, J=14.6, 6.2 Hz, 2H), 2.43-2.35 (m, 2H), 1.46-1.38 (m, 2H).

### Example 59 7′-bromo-4-(3-bromoanilino)spiro[cyclohexane-1,1′-indene]-4-carboxylic acid, diastereoisomer 1

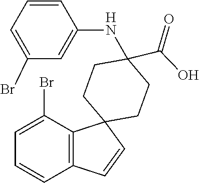

Using General procedure 10 and Example 59C (130 mg, 0.47 mmol 2 eq) as the appropriate ketone and 3-bromoaniline (40 mg, 26 uL, 0.24 mmol, 1 eq) as the appropriate aniline, a single diastereoisomer, Example 59 was isolated as a white solid (7 mg, 0.01 mmol, 6%). LRMS calculated for C₂₁H₁₉Br₂NO₂: 475; found: 476 (M+H). ¹H NMR (400 MHz, DMSO-d₆) δ ppm: 12.93 (br s, 1H), 7.38-7.31 (m, 2H), 7.20-7.14 (m, 2H), 7.01 (t, J=8.1 Hz, 1H), 6.91 (t, J=2.1 Hz, 1H), 6.77 (d, J=5.7 Hz, 1H), 6.74-6.67 (m, 2H), 6.19 (br s, 1H), 3.05-2.92 (m, 2H), 2.67-2.58 (m, 2H), 1.84-1.71 (m, 2H), 1.12-1.02 (m, 2H).

### Example 60

### Example 60A 4′-bromospiro[cyclohexane-1,1′-inden]-4-one

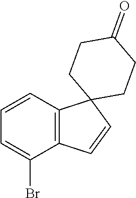 [SMILES: O=C1CCC2(C=Cc3c(Br)cccc32)CC1]

Using General procedure 9 and Example 59B (100 mg, 0.31 mmol, 1 eq) as the appropriate ketal in THE (4 mL), Example 60A was isolated as a yellow solid (86 mg, 0.31 mmol, 100%). ¹H NMR (400 MHz, DMSO-d₆) δ ppm: 7.52 (dt, J=7.5, 0.8 Hz, 1H), 7.46-7.41 (m, 2H), 7.15 (t, J=7.7 Hz, 1H), 6.88 (d, J=5.7 Hz, 1H), 2.82-2.70 (m, 2H), 2.44-2.30 (m, 4H), 1.57-1.47 (m, 2H).

### Example 60 (1r,4r)-4′-bromo-4-(3-bromoanilino)spiro[cyclohexane-1,1′-indene]-4-carboxylic acid

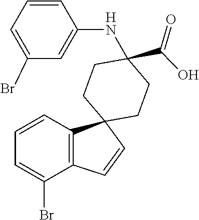

Using General procedure 10 and Example 60A (90 mg, 0.32 mmol, 1 eq) as the appropriate ketone and 3-bromoaniline (55 mg, 35 uL, 0.32 mmol, 1 eq) as the appropriate aniline, a single diastereoisomer, Example 60 was isolated as a white solid (20 mg, 0.04 mmol, 13%). LRMS calculated for C₂₁H₁₉Br₂NO₂: 475; found: 476 (M+H). ¹H NMR (400 MHz, DMSO-d₆) δ ppm: 12.88 (br s, 1H), 7.44 (d, J=8.0 Hz, 1H), 7.35 (d, J=7.4 Hz, 1H), 7.16 (t, J=7.8 Hz, 1H), 7.02 (t, J=8.1 Hz, 1H), 6.99 (d, J=5.7 Hz, 1H), 6.84 (t, J=2.1 Hz, 1H), 6.77 (d, J=5.7 Hz, 1H), 6.74-6.69 (m, 1H), 6.67-6.62 (m, 1H), 6.28 (br s, 1H), 1.97-1.76 (m, 4H), 1.59-1.47 (m, 2H).

### Example 61 and Example 62

### Example 61A 6-methoxy-1-(methoxymethylidene)-2,3-dihydro-1H-indene

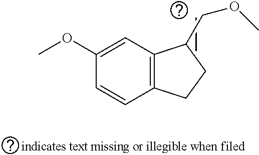

To a stirred suspension of KOtBu (1.59 mg, 14.2 mmol, 2.3 eq) in 1,4-dioxane (40 mL) was added (methoxymethyl)triphenylphosphonium chloride (10.1 g, 29.4 mmol, 2.3 eq) under N₂ and the mixture stirred for 2 h at rt. A solution of 6-methoxy-1-indanone (2.08 g, 12.8 mmol, 1 eq) in 1,4-dioxane (40 mL) was added and the mixture was stirred at rt for 18 h. The reaction mixture was diluted with water and extracted with EtOAc. The combined organic extracts were washed with water, brine, dried (MgSO₄) and concentrated in vacuo. Purification by automated flash column chromatography (CombiFlash Rf, 120 g RediSep™ silica cartridge) eluting with a gradient of 0-10% EtOAc in heptane afforded a mixture of E/Z isomers, Example 61A as a colourless liquid (1.4 g, 7.35 mmol, 57%). LRMS calculated for Cl₂H₁₄O₂: 190; found: 191 (M+H). ¹H NMR (400 MHz, DMSO-d₆) δ ppm: 7.22/6.94 (d, 1H), 7.10/7.06 (d, 1H), 6.92/6.29 (t, 1H), 6.68/6.62 (dd, 1H), 3.72/3.71 (s, 3H), 3.69/3.68 (s, 3H), 2.85-2.79 (m, 2H), 2.66-2.59 (m, 2H).

### Example 61B 6′-methoxy-2′,3′-dihydrospiro[cyclohex-2-ene-1,1′-inden]-4-one

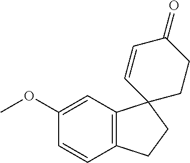 [SMILES: COc1ccc2c(c1)C1(C=CC(=O)CC1)CC2]

To a solution of Example 61A (1.4 g, 7.35 mmol, 1 eq) in toluene (13 mL) was added MVK (0.71 mL, 8.08 mmol, 1.1 eq) and PTSA (140 mg, 0.73 mmol, 0.1 eq) and the mixture was stirred at 85° C. for 18 h under N₂. The mixture was allowed to cool to rt and poured into sat. aq. NaHCO₃ solution. The organic layer was separated, and the aq. phase was extracted with EtOAc. The combined organic extracts were washed with water, brine, dried (MgSO₄) and concentrated in vacuo. Purification by automated flash column chromatography (CombiFlash Rf, 40 g RediSep™ silica cartridge) eluting with a gradient of 0-10% EtOAc in heptane afforded racemate Example 61B as a yellow oil (1 g, 4.37 mmol, 59%). ¹H NMR (400 MHz, DMSO-d₆) δ ppm: 7.19 (d, J=8.3 Hz, 1H), 6.87 (d, J=10.1 Hz, 1H), 6.79 (dd, J=8.2, 2.5 Hz, 1H), 6.65 (d, J=2.4 Hz, 1H), 5.99 (d, J=10.1 Hz, 1H), 3.72 (s, 3H), 3.00-2.80 (m, 2H), 2.43-2.26 (m, 2H), 2.15-2.01 (m, 3H).

### Example 61C 6′-methoxy-2′,3′-dihydrospiro[cyclohexane-1,1′-inden]-4-one

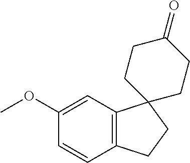 [SMILES: COc1ccc2c(c1)C1(CCC(=O)CC1)CC2]

To a solution of Example 61B (1 g, 4.37 mmol, 1 eq) in EtOH (50 mL) was added 10% Pd/C (100 mg). The flask was evacuated and backfilled with N₂ (×3), then evacuated and filled with H₂. The reaction mixture was shaken at rt under an atmosphere of H₂ for 18 h. The reaction mixture was filtered, concentrated under vacuum and the residue was taken up in AcOH (50 mL). 10% Pd/C (100 mg) was added and the flask was evacuated and backfilled with N₂ (×3), then evacuated and filled with H₂. The reaction mixture was shaken at rt under an atmosphere of H₂ for 4 h after which time no more conversion was observed. The reaction mixture was filtered, concentrated and dried in vacuo to afford Example 61C as a white solid (1.01 g, 4.37 mmol, 100%). ¹H NMR (400 MHz, DMSO-d₆) δ ppm: 7.13-7.09 (m, 1H), 6.83 (d, J=2.4 Hz, 1H), 6.72 (dd, J=8.2, 2.4 Hz, 1H), 3.71 (s, 3H), 2.85 (t, J=7.3 Hz 2H), 2.61 (td, J=14.3, 6.0 Hz, 2H), 2.26-2.18 (m, 4H), 2.10-2.00 (m, 2H), 1.83-1.74 (m, 2H).

### Example 61 (1r,4s)-4-(3-bromoanilino)-6′-methoxy-2′,3′-dihydrospiro[cyclohexane-1,1′-indene]-4-carboxylic acid

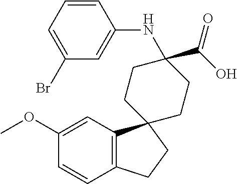 [SMILES: COc1ccc2c(c1)[C@]1(CC2)CC[C@](Nc2cccc(Br)c2)(C(=O)O)CC1]

and

### Example 62 (1s,4r)-4-(3-bromoanilino)-6′-methoxy-2′,3′-dihydrospiro[cyclohexane-1,1′-indene]-4-carboxylic acid

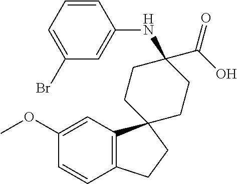 [SMILES: COc1ccc2c(c1)[C@]1(CC2)CC[C@@](Nc2cccc(Br)c2)(C(=O)O)CC1]

Using General procedure 10 and Example 61C (98 mg, 0.38 mmol, 1 eq) as the appropriate ketone and 3-bromoaniline (42 μL, 0.38 mmol, 1 eq) as the appropriate aniline, a mixture of diastereoisomers was obtained. The diastereoisomers were separated by automated flash column chromatography (CombiFlash Rf, 24 g RediSep™ silica cartridge) eluting with a gradient of 0-65% EtOAc in heptane. The diastereoisomer eluting earlier was collected as Example 61 (white solid, 45 mg, 0.1 mmol, 27%). LRMS calculated for C₂₂H₂₄BrNO₃: 429; found: 430 (M+H). ¹H NMR (400 MHz, DMSO-d₆) δ ppm: 7.10 (d, J=8.2 Hz, 1H), 7.00 (t, J=8.1 Hz, 1H), 6.83 (t, J=2.0 Hz, 1H), 6.74-6.66 (m, 2H), 6.65-6.58 (m, 2H), 3.72 (s, 3H), 2.77 (t, J=7.3 Hz, 2H), 2.45-2.36 (m, 2H), 1.98 (t, J=7.3 Hz, 2H), 1.82-1.72 (m, 2H), 1.71-1.61 (m, 2H), 1.57-1.49 (m, 2H).

The diastereoisomer eluting later was collected as Example 62 (white solid, 5.5 mg, 0.01 mmol, 3.3%). LRMS calculated for C₂₂H₂₄BrNO₃: 429; found: 430 (M+H). ¹H NMR (400 MHz, DMSO-d₆) δ ppm: 12.63 (br s, 1H), 7.09 (d, J=8.1 Hz, 1H), 7.02 (t, J=8.1 Hz, 1H), 6.81 (t, J=2.1 Hz, 1H), 6.74-6.65 (m, 3H), 6.62-6.57 (m, 1H), 6.18 (br s, 1H), 3.72 (s, 3H), 2.77 (t, J=7.3 Hz, 2H), 2.13-2.05 (m, 2H), 2.02-1.84 (m, 6H), 1.37-1.28 (m, 2H).

### Example 63 (1r,4s)-4-(3-bromoanilino)-6′-hydroxy-2′,3′-dihydrospiro[cyclohexane-1,1′-indene]-4-carboxylic acid

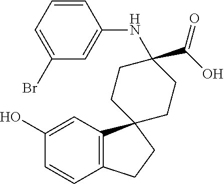 [SMILES: O=C(O)[C@]1(Nc2cccc(Br)c2)CC[C@@]2(CCc3ccc(O)cc32)CC1]

To a solution of Example 61 in DCM (2 mL), cooled to −78° C., was added BBr₃ (1 M in DCM, 312 μL, 0.31 mmol, 4 eq). The mixture was stirred at rt for 18 h and then cooled to −78° C., quenched with MeOH and then stirred at rt. The solvent was removed in vacuo and the residue dissolved in MeOH (containing a few drops of TEA), loaded onto a MeOH-wet PE-AX cartridge (5 g) and the column was washed successively with MeOH, DCM and eluted with 10% HCOOH in DCM to afford Example 63 as a white solid (12.3 mg, 0.03 mmol, 38%). LRMS calculated for C₂₁H₂₂BrNO₃: 415; found: 416 (M+H). ¹H NMR (400 MHz, DMSO-d₆) δ ppm: 6.98-6.92 (m, 2H), 6.87 (t, J=2.1 Hz, 1H), 6.68-6.60 (m, 2H), 6.54-6.50 (m, 2H), 2.71 (t, J=7.2 Hz, 2H), 2.42-2.32 (m, 2H), 1.94 (t, J=7.2 Hz, 2H), 1.85-1.74 (m, 2H), 1.63-1.52 (m, 2H), 1.50-1.41 (m, 2H).

### Example 64 and Example 65 Example 64A dispiro[[1,3]dioxolane-2,1′-cyclohexane-4′,1″-indene]

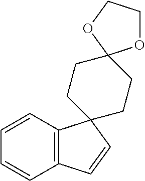 [SMILES: C1=CC2(CCC3(CC2)OCCO3)c2ccccc21]

Using General procedure 8a and 1H-indene as the appropriate indene Example 64A was obtained. LRMS calculated for C₁₆H₁₈O₂: 242.1; found 243.0 (M+H).

### Example 64B spiro[cyclohexane-1,1′-inden]-4-one

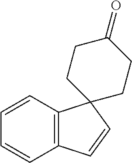 [SMILES: O=C1CCC2(C=Cc3ccccc32)CC1]

Using General procedure 9 and Example 64A as the appropriate ketal Example 64B was obtained. ¹H NMR (500 MHz, DMSO-d₆) δ ppm: 7.50 (d, 1H), 7.47 (d, 1H), 7.36 (d, 1H), 7.24 (td, 1H), 7.17 (td, 1H), 6.89 (d, 1H), 2.74/2.36 (td+m, 4H), 2.34/1.48 (td+m, 4H). HRMS calculated for C₁₄H₁₄O: 198.1045; found 199.1124 (M+H).

### Example 64 (1r,4r)-4-(3-chloroanilino)spiro[cyclohexane-1,1′-indene]-4-carboxylic acid

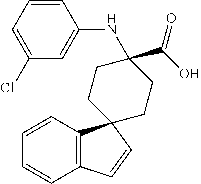 [SMILES: O=C(O)[C@]1(Nc2cccc(Cl)c2)CC[C@@]2(C=Cc3ccccc32)CC1]

and

### Example 65 (1s,4s)-4-(3-chloroanilino)spiro[cyclohexane-1,1′-indene]-4-carboxylic acid

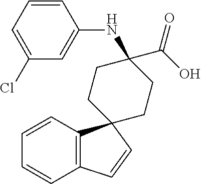 [SMILES: O=C(O)[C@]1(Nc2cccc(Cl)c2)CC[C@]2(C=Cc3ccccc32)CC1]

Using General procedure 10 and Example 64B as the appropriate ketone a mixture of diastereoisomers was obtained. The diastereoisomers were separated via flash chromatography using heptane and EtOAc as eluents. The diastereoisomer eluting earlier was collected as Example 64. HRMS calculated for C₂₁H₂₀NO₂Cl: 353.1183; found: 354.1260 (M+H). The diastereoisomer eluting later was collected as Example 65. HRMS calculated for C₂₁H₂₀NO₂Cl: 353.1183; found: 354.1250 (M+H).

### Example 67

### Example 67A 2-methoxyethyl (1r,4s)-4-(3-bromoanilino)-6′-(2-methoxyethoxy)-2′,3′-dihydrospiro[cyclohexane-1,1′-indene]-4-carboxylate

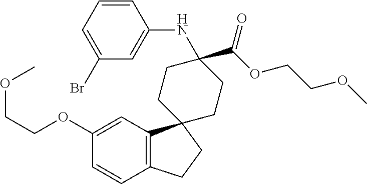 [SMILES: COCCOc1ccc2c(c1)[C@]1(CC2)CC[C@](Nc2cccc(Br)c2)(C(=O)OCCOC)CC1]

To a solution of Example 63 (107 mg, 0.26 mmol, 1 eq) in a mixture of acetone (5 mL) and THE (4 mL) was added NaH (60% dispersion; 26 mg, 0.64 mmol, 2.5 eq) with rapid stirring. 2-bromoethyl methyl ether (61 μL, 0.64 mmol, 2.5 eq) and KI (68 mg, 0.41 mmol, 1.6 eq) were added and the reaction was heated at 70° C. for 18 h. After cooling, the mixture was partitioned between DCM and brine. The organic phase was separated, dried (MgSO₄) and concentrated in vacuo. Purification by automated flash chromatography (CombiFlash Rf, 24 g RediSep™ silica cartridge) eluting with a gradient of 0-10% EtOAc in DCM afforded Example 67A as a colourless oil (30 mg, 0.06 mmol, 22%). LRMS calculated for C₂₇H₃₄BrNO₅: 531; found: 532 (M+H). ¹H NMR (400 MHz, DMSO-d₆) δ ppm: 7.09 (d, J=8.2 Hz, 1H), 7.00 (t, J=8.1 Hz, 1H), 6.80 (t, J=2.1 Hz, 1H), 6.75-6.66 (m, 3H), 6.61-6.56 (m, 1H), 6.24 (s, 1H), 4.32-4.27 (m, 2H), 4.07-4.01 (m, 2H), 3.67-3.62 (m, 2H), 3.55-3.49 (m, 2H), 3.31 (s, 3H), 3.22 (s, 3H), 2.77 (t, J=7.2 Hz, 2H), 2.47-2.40 (m, 2H), 1.98 (t, J=7.2 Hz, 2H), 1.82-1.64 (m, 4H), 1.57-1.48 (m, 2H).

### Example 67 (1r,4s)-4-(3-bromoanilino)-6′-(2-methoxyethoxy)-2′,3′-dihydrospiro[cyclohexane-1,1′-indene]-4-carboxylic acid

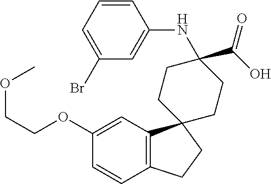 [SMILES: COCCOc1ccc2c(c1)[C@]1(CC2)CC[C@](Nc2cccc(Br)c2)(C(=O)O)CC1]

To a solution of Example 67A (30 mg, 0.6 mmol, 1 eq) in MeOH (3 mL) was added 1 M aq. NaOH solution (0.28 mL, 0.28 mmol, 5.0 eq) and the mixture heated under microwave irradiation at 120° C. for 1.5 h. The mixture was concentrated in vacuo and the residue was suspended in minimal water. The pH was adjusted to 6 by the careful addition of 2 M aq. HCl solution and extracted with DCM. The organic phase was separated, washed with brine, dried (MgSO₄) and concentrated in vacuo. The residue was dissolved in MeOH containing a few drops of TEA and then loaded onto a MeOH-wet PE-AX cartridge (10 g). The column was washed successively with MeOH, DCM and eluted with 10% HCOOH in DCM to afford Example 67 as a white solid (23 mg, 0.05 mmol, 86%). LRMS calculated for C₂₄H₂₈BrNO₄: 473; found: 474 (M+H). ¹H NMR (400 MHz, DMSO-d₆) δ ppm: 7.09 (d, J=8.3 Hz, 1H), 7.00 (t, J=8.1 Hz, 1H), 6.83 (t, J=2.1 Hz, 1H), 6.75-6.66 (m, 2H), 6.66-6.59 (m, 2H), 4.07-4.01 (m, 2H), 3.67-3.62 (m, 2H), 3.31 (s, 3H), 2.77 (t, J=7.2 Hz, 2H), 2.44-2.36 (m, 2H), 1.98 (t, J=7.2 Hz, 2H), 1.83-1.72 (m, 2H), 1.72-1.61 (m, 2H), 1.58-1.49 (m, 2H).

### Example 68

### Example 68A prop-2-en-1-yl (1r,4s)-4-(3-bromoanilino)-6′-[(prop-2-en-1-yl)oxy]-2′,3′-dihydrospiro[cyclohexane-1,1′-indene]-4-carboxylate

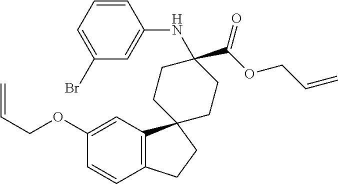 [SMILES: C=CCOc1ccc2c(c1)[C@]1(CC2)CC[C@](Nc2cccc(Br)c2)(C(=O)OCC=C)CC1]

To a solution of Example 63 (190 mg, 0.46 mmol, 1 eq) in DMF (5 mL), cooled to 0° C., was added NaH (60% dispersion; 26 mg, 1 mmol, 2.2 eq) and stirring continued at 0° C. for 30 min. Allyl bromide (87 μL, 1 mmol, 2.2 eq) was added and the reaction was stirred at rt for 18 h. The mixture was partitioned between DCM and water and the aq. phase was extracted with DCM. The combined organic extracts were dried (MgSO₄) and concentrated in vacuo. Purification by automated flash chromatography (CombiFlash Rf, 40 g RediSep™ silica cartridge) eluting with a gradient of 0-10% EtOAc in DCM afforded Example 68A as a solid (125 mg, 0.25 mmol, 55%). ¹H NMR (400 MHz, DMSO-d₆) δ ppm: 7.09 (d, J=8.2 Hz, 1H), 7.00 (t, J=8.1 Hz, 1H), 6.80 (t, J=2.1 Hz, 1H), 6.76-6.69 (m, 2H), 6.65 (d, J=2.4 Hz, 1H), 6.61-6.56 (m, 1H), 6.26 (s, 1H), 6.09-5.99 (m, 1H), 5.94-5.83 (m, 1H), 5.43-5.37 (m, 1H), 5.34-5.24 (m, 2H), 5.22-5.18 (m, 1H), 4.67 (dt, J=5.5, 1.4 Hz, 2H), 4.52 (dt, J=5.2, 1.6 Hz, 2H), 2.77 (t, J=7.2 Hz, 2H), 2.47-2.40 (m, 2H), 1.98 (t, J=7.2 Hz, 2H), 1.78-1.66 (m, 4H), 1.61-1.49 (m, 2H).

### Example 68 (1r,4s)-4-(3-bromoanilino)-6′-[(prop-2-en-1-yl)oxy]-2′,3′-dihydrospiro[cyclohexane-1,1′-indene]-4-carboxylic acid

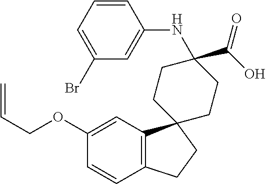 [SMILES: C=CCOc1ccc2c(c1)[C@]1(CC2)CC[C@](Nc2cccc(Br)c2)(C(=O)O)CC1]

To a solution of Example 68A (125 mg, 0.25 mmol, 1 eq) in MeOH (15 mL) was added 1 M aq. NaOH solution (2.52 mL, 2.52 mmol, 10 eq) and the mixture stirred at rt for 64 h. Water (1 mL) was added and the reaction was heated at 50° C. for 72 h. After cooling to rt, the mixture was concentrated in vacuo and the residue was suspended in minimal water. The pH was adjusted to 6 by the careful addition of 2 M aq. HCl solution and extracted with DCM. The organic phase was separated, washed with brine, dried (MgSO₄) and concentrated in vacuo to afford Example 68 as a white solid (114 mg, 0.25 mmol, 99%). LRMS calculated for C₂₄H₂₆BrNO₃: 455; found: 456 (M+H). ¹H NMR (400 MHz, DMSO-d₆) δ ppm: 7.08 (d, J=8.2 Hz, 1H), 6.97 (t, J=8.0 Hz, 1H), 6.88-6.84 (m, 1H), 6.73 (dd, J=8.2, 2.4 Hz, 1H), 6.68-6.61 (m, 3H), 6.08-5.99 (m, 1H), 5.39 (dq, J=17.3, 1.7 Hz, 1H), 5.25 (dq, J=10.5, 1.5 Hz, 1H), 4.52 (dt, J=5.2, 1.6 Hz, 2H), 2.76 (t, J=7.2 Hz, 2H), 2.43-2.35 (m, 2H), 1.97 (t, J=7.3 Hz, 2H), 1.87-1.74 (m, 2H), 1.69-1.55 (m, 2H), 1.55-1.42 (m, 2H).

### Example 69 (1r,4s)-4-(3-chloroanilino)-2′,3′-dihydrospiro[cyclohexane-1,1′-indene]-4-carboxylic acid

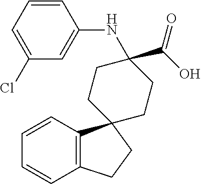 [SMILES: O=C(O)[C@]1(Nc2cccc(Cl)c2)CC[C@@]2(CCc3ccccc32)CC1]

Example 75 (86 mg, 0.2 mmol, 1 eq) was dissolved in EtOH (20 mL) and hydrogenated using an H-Cube Pro at 5 bar and 30° C. using a 5% Pt/C cartridge. After completion of the cycle the solvent was concentrated in vacuo. Purification by automated flash chromatography (CombiFlash Rf, 4 g RediSep™ silica cartridge) eluting with a gradient of 0-50% EtOAc in heptane afforded Example 69 as a solid (19.8 mg, 0.06 mmol, 28%). LRMS calculated for C₂₁H₂₂ClNO₂: 355; found: 356 (M+H). ¹H NMR (400 MHz, DMSO-d₆) δ ppm: 7.23-7.03 (m, 5H), 6.68 (t, J=2.1 Hz, 1H), 6.62-6.58 (m, 1H), 6.58-6.54 (m, 1H), 2.85 (t, J=7.3 Hz, 2H), 2.46-2.37 (m, 2H), 1.98 (t, J=7.3 Hz, 2H), 1.85-1.74 (m, 2H), 1.73-1.62 (m, 2H), 1.59-1.50 (m, 2H).

### Example 70

### Example 70A 6,7-dihydro-5H-cyclopenta[b]pyridin-7-ol

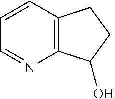

To a stirred solution of 5,6-dihydro-7H-cyclopenta[b]pyridin-7-one (1.05 g, 7.9 mmol) in MeOH (25 mL) was added NaBH₄ (0.30 g, 7.9 mmol) portionwise at 10° C. and then stirring continued at rt for 1 h. The solvent was removed in vacuo and purification by automated flash chromatography (CombiFlash Rf, 40 g RediSep™ silica cartridge) eluting with a gradient of 0-5% MeOH in DCM afforded racemate Example 70A as a pink oil that solidified on further drying in vacuo (910 mg, 6.73 mmol, 85%). LRMS calculated for C₈H₉NO: 135; found: 136 (M+H). ¹H NMR (400 MHz, DMSO-d₆) δ ppm: 8.42-8.36 (m, 1H), 7.67-7.62 (m, 1H), 7.20 (dd, J=7.6, 4.9 Hz, 1H), 5.34 (d, J=5.6 Hz, 1H), 4.97-4.90 (m, 1H), 2.99-2.90 (m, 1H), 2.78-2.69 (m, 1H), 2.39-2.29 (m, 1H), 1.87-1.78 (m, 1H).

### Example 70B 5H-cyclopenta[b]pyridine

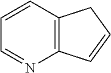

Example 70A (900 mg, 6.66 mmol) was added to a mixture of cc. H₂SO₄ (10 mL) and water (10 mL) and then heated at 140° C. for 18 h and then a further 96 h at rt. The mixture was carefully added to rapidly stirring ice/water and then adjusted to pH 10 by the careful addition of 4 M aq. NaOH solution. The aq. phase was extracted with EtOAc and the combined organic extracts were washed with water and brine, dried (MgSO₄) and concentrated in vacuo to afford Example 70B as a volatile oil (780 mg, 6.66 mmol, 100%) that was used immediately without any further purification or drying. LRMS calculated for C₈H₇N: 117; found: 118 (M+H).

### Example 70C dispiro[cyclopenta[b]pyridine-5,1′-cyclohexane-4′,2″-[1,3]dioxolane]

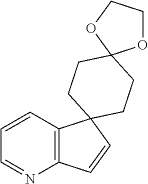 [SMILES: C1=CC2(CCC3(CC2)OCCO3)c2cccnc21]

and

### Example 70D dispiro[cyclopenta[b]pyridine-7,1′-cyclohexane-4′,2″-[1,3]dioxolane]

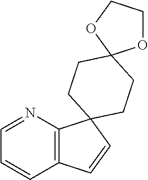 [SMILES: C1=CC2(CCC3(CC2)OCCO3)c2ncccc21]

Using General procedure 8a and Example 70B (0.78 g, 6.66 mmol) as the appropriate indene, a mixture of regioisomers was obtained, which were separated by automated flash chromatography (CombiFlash Rf, 40 g RediSep™ silica cartridge) eluting with a gradient of 0-40% EtOAc in heptane. The regioisomer eluting later was collected as Example 70C as a pink solid (254 mg, 0.25 mmol, 13%). LRMS calculated for C₁₅H₁₇NO₂: 243; found: 244 (M+H). ¹H NMR (400 MHz, DMSO-d₆) δ ppm: 8.35 (dd, J=5.0, 1.5 Hz, 1H), 7.75 (dd, J=7.5, 1.5 Hz, 1H), 7.40 (d, J=5.8 Hz, 1H), 7.12 (dd, J=7.5, 5.0 Hz, 1H), 6.86 (d, J=5.7 Hz, 1H), 4.02-3.90 (m, 4H), 2.13-2.01 (m, 2H), 1.93-1.78 (m, 4H), 1.35-1.25 (m, 2H).

The regioisomer eluting earlier was collected as Example 70D as a pink solid (680 mg, 0.58 mmol, 31%). LRMS calculated for C₁₅H₁₇NO₂: 243; found: 244 (M+H). ¹H NMR (400 MHz, DMSO-d6) δ ppm: 8.29 (dd, J=5.0, 1.5 Hz, 1H), 7.69 (dd, J=7.5, 1.5 Hz, 1H), 7.20 (dd, J=7.5, 5.0 Hz, 1H), 7.01 (d, J=6.0 Hz, 1H), 6.85 (d, J=6.0 Hz, 1H), 4.03-3.87 (m, 4H), 2.12-1.94 (m, 4H), 1.85-1.74 (m, 2H), 1.41-1.28 (m, 2H).

### Example 70E spiro[cyclohexane-1,5′-cyclopenta[b]pyridin]-4-one

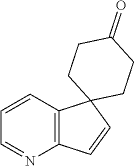 [SMILES: O=C1CCC2(C=Cc3ncccc32)CC1]

Using General procedure 9 and Example 70C (50 mg, 0.21 mmol) as the appropriate ketal, Example 70E was obtained as a pink solid (150 mg, 0.75 mmol, 72%). LRMS calculated for C₁₃H₁₃NO: 199; found: 200 (M+H). ¹H NMR (400 MHz, DMSO-d₆) δ ppm: 8.39 (dd, J=5.0, 1.5 Hz, 1H), 7.90 (ddd, J=7.5, 1.5, 0.6 Hz, 1H), 7.63 (d, J=5.7 Hz, 1H), 7.15 (dd, J=7.5, 5.0 Hz, 1H), 6.96 (d, J=5.8 Hz, 1H), 2.82-2.69 (m, 2H), 2.47-2.31 (m, 4H), 1.62-1.52 (m, 2H).

### Example 70 (1r,4r)-4-(3-chloroanilino)spiro[cyclohexane-1,5′-cyclopenta[b]pyridine]-4-carboxylic acid

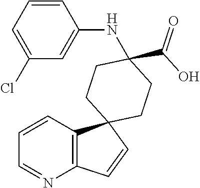 [SMILES: O=C(O)[C@]1(Nc2cccc(Cl)c2)CC[C@@]2(C=Cc3ncccc32)CC1]

Using General procedure 10 and Example 70E (140 mg, 0.70 mmol) as the appropriate ketone and 3-chloroaniline (73 uL, 0.70 mmol, 1 eq) as the appropriate aniline, a mixture of diastereoisomers was obtained as a solid (30 mg, 84.55 μmol, 12%). The diastereoisomers were separated by chiral chromatography. Column: AD, 50×500 mm, 20 μm. Eluents: 30:70 iPrOH/Heptane+0.05% HCOOH. The diastereoisomer eluting earlier was collected as Example 70 (7.3 mg, 0.02 mmol, 24%). ¹H NMR (500 MHz, DMSO-d₆) δ ppm: 8.39 (d, 1H), 7.73 (d, 1H), 7.16 (dd, 1H), 7.14 (d, 1H), 7.06 (t, 1H), 6.82 (d, 1H), 6.72 (m, 1H), 6.63 (m, 1H), 6.55 (m, 1H), 2.42/1.94 (m, 4H), 1.70/1.63 (m, 4H). HRMS calculated for C₂₀H₁₉N₂O₂Cl: 354.1135; found: 355.1211 (M+H).

### Example 71

### Example 71A spiro[cyclohexane-1,7′-cyclopenta[b]pyridin]-4-one

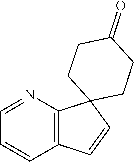 [SMILES: O=C1CCC2(C=Cc3cccnc32)CC1]

Using General procedure 9 and Example 70D (680 mg, 2.79 mmol) as the appropriate ketal, Example 71A was obtained as an indigo solid (474 mg, 2.38 mmol, 85%). LRMS calculated for C₁₃H13NO: 199; found: 200 (M+H). ¹H NMR (400 MHz, DMSO-d₆) δ ppm: 8.32 (dd, J=5.0, 1.6 Hz, 1H), 7.75 (dd, J=7.6, 1.5 Hz, 1H), 7.26 (dd, J=7.6, 5.0 Hz, 1H), 7.12 (d, J=6.0 Hz, 1H), 6.96 (d, J=6.0 Hz, 1H), 2.77-2.67 (m, 2H), 2.67-2.56 (m, 2H), 2.17-2.06 (m, 2H), 1.81-1.71 (m, 2H).

### Example 71B 4-(3-chloroanilino)spiro[cyclohexane-1,7′-cyclopenta[b]pyridine]-4-carbonitrile

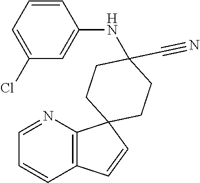 [SMILES: N#CC1(Nc2cccc(Cl)c2)CCC2(C=Cc3cccnc32)CC1]

Using General procedure 11 and Example 71A (300 mg, 1.51 mmol, 1 eq) as the appropriate ketone and 3-chloroaniline (158 μL, 1.51 mmol, 1 eq) as the appropriate aniline, a 2:1 mixture of diastereoisomers, Example 71B was obtained as a white solid (350 mg, 1.04 mmol, 69%). LRMS calculated for C₂₀H₁₈N₃Cl: 335; found: 336 (M+H). ¹H NMR (400 MHz, DMSO-d₆) δ ppm: 8.38-8.27 (m, 1H), 7.75-7.69 (m, 1H), 7.28-7.20 (m, 2H), 7.06-6.85 (m, 4H), 6.80-6.74 (m, 1H), 6.62/6.52 (s, 1H), 2.73-2.59 (m, 2H), 2.41-1.37 (m, 6H).

### Example 71C 4-(3-chloroanilino)spiro[cyclohexane-1,7′-cyclopenta[b]pyridine]-4-carboxamide, diastereoisomer 1

### Example 71D 4-(3-chloroanilino)spiro[cyclohexane-1,7′-cyclopenta[b]pyridine]-4-carboxamide, diastereoisomer 2

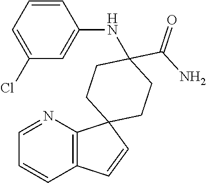 [SMILES: NC(=O)C1(Nc2cccc(Cl)c2)CCC2(C=Cc3cccnc32)CC1]

Using General procedure 12b and Example 71B (210 mg, 0.31 mmol, 1 eq) as the appropriate nitrile, a mixture of diastereoisomers was obtained, which were separated by automated flash chromatography (CombiFlash Rf, 24 g RediSep™ silica cartridge) eluting with a gradient of 0-50% EtOAc in DCM. The diastereoisomer eluting earlier was collected as Example 71C (80 mg, 0.23 mmol, 36%). LRMS calculated for C₂₀H₂₀N₃OCl: 353; found: 354 (M+H). ¹H NMR (400 MHz, DMSO-d₆) δ ppm: 8.29 (dd, J=5.0, 1.6 Hz, 1H), 7.68 (dd, J=7.6, 1.6 Hz, 1H), 7.34 (s, 1H), 7.21 (dd, J=7.5, 5.0 Hz, 1H), 7.11 (s, 1H), 7.08 (t, J=8.1 Hz, 1H), 6.82 (d, J=5.9 Hz, 1H), 6.75-6.70 (m, 2H), 6.66-6.57 (m, 2H), 5.97 (s, 1H), 2.78-2.66 (m, 2H), 1.91-1.79 (m, 2H), 1.75-1.60 (m, 4H).

The diastereoisomer eluting later was collected as Example 71D (145 mg, 0.41 mmol, 66%). LRMS calculated for C₂₀H₂₀N₃0Cl: 353; found: 354 (M+H). ¹H NMR (400 MHz, DMSO-d₆) δ ppm: 8.30 (dd, J=5.0, 1.5 Hz, 1H), 7.69 (dd, J=7.5, 1.5 Hz, 1H), 7.30 (s, 1H), 7.21 (dd, J=7.5, 5.0 Hz, 1H), 7.13 (s, 1H), 7.10 (t, J=8.1 Hz, 1H), 7.05 (d, J=6.1 Hz, 1H), 6.88 (d, J=6.1 Hz, 1H), 6.74 (t, J=2.1 Hz, 1H), 6.66-6.57 (m, 2H), 6.16 (s, 1H), 2.36-2.24 (m, 2H), 2.22-2.04 (m, 4H), 1.08-0.99 (m, 2H).

### Example 71 4-(3-chloroanilino)spiro[cyclohexane-1,7′-cyclopenta[b]pyridine]-4-carboxylic acid, diastereoisomer 1

and

### Example 72 4-(3-chloroanilino)spiro[cyclohexane-1,7′-cyclopenta[b]pyridine]-4-carboxylic acid, diastereoisomer 2

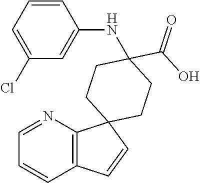 [SMILES: O=C(O)C1(Nc2cccc(Cl)c2)CCC2(C=Cc3cccnc32)CC1]

Using General procedure 33b and Example 71C (100 mg, 0.29 mmol), single diastereoisomer Example 71 was obtained as a white solid (17 mg, 0.05 mmol, 17%). LRMS calculated for C₂₀H₁₉N₂O₂Cl: 354; found: 355 (M+H). ¹H NMR (400 MHz, DMSO-d₆) δ ppm: 12.73 (br s, 1H), 8.30 (dd, J=5.0, 1.5 Hz, 1H), 7.69 (dd, J=7.6, 1.6 Hz, 1H), 7.22 (dd, J=7.5, 5.0 Hz, 1H), 7.08 (t, J=8.1 Hz, 1H), 6.84 (d, J=5.9 Hz, 1H), 6.79 (d, J=6.0 Hz, 1H), 6.70 (t, J=2.1 Hz, 1H), 6.65-6.54 (m, 2H), 6.25 (br s, 1H), 2.80-2.69 (m, 2H), 1.99-1.88 (m, 2H), 1.77-1.60 (m, 4H).

Using General procedure 33b and Example 71D (125 mg, 0.35 mmol), single diastereoisomer Example 72 was obtained as a white solid (60 mg, 0.16 mmol, 48%). LRMS calculated for C₂₀H₁₉N₂O₂Cl: 354; found: 355 (M+H). ¹H NMR (400 MHz, DMSO-d₆) δ ppm: 12.70 (br s, 1H), 8.29 (dd, J=5.0, 1.5 Hz, 1H), 7.69 (dd, J=7.6, 1.5 Hz, 1H), 7.21 (dd, J=7.5, 5.0 Hz, 1H), 7.12-7.06 (m, 2H), 6.88 (d, J=6.1 Hz, 1H), 6.66 (t, J=2.1 Hz, 1H), 6.63-6.55 (m, 2H), 6.37 (br s, 1H), 2.38-2.24 (m, 4H), 2.15-2.03 (m, 2H), 1.13-1.02 (m, 2H).

### Example 73

### Example 73A methyl (1r,4r)-4-(3-chloroanilino)spiro[cyclohexane-1,1′-indene]-4-carboxylate

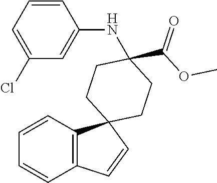 [SMILES: COC(=O)[C@]1(Nc2cccc(Cl)c2)CC[C@@]2(C=Cc3ccccc32)CC1]

Using General procedure 17a and Example 64 as the appropriate amino acid Example 73A was obtained. ¹H NMR (500 MHz, DMSO-d₆) δ ppm: 7.4 (d, 1H), 7.36 (d, 1H), 7.26 (t, 1H), 7.21 (t, 1H), 7.11 (t, 1H), 6.83 (d, 1H), 6.8 (d, 1H), 6.68 (t, 1H), 6.62 (dd, 1H), 6.56 (dd, 1H), 6.38 (s, 1H), 3.74 (s, 3H), 2.56/1.97 (d+t, 4H), 1.81/1.53 (t+d, 4H). HRMS calculated for C₂₂H₂₂ClNO₂: 367.1339; found 368.1416 (M+H).

### Example 73B methyl (1r,4r)-4-[(3-chlorophenyl)(methyl)amino]spiro[cyclohexane-1,1′-indene]-4-carboxylate

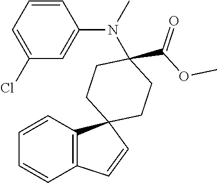 [SMILES: COC(=O)[C@]1(N(C)c2cccc(Cl)c2)CC[C@@]2(C=Cc3ccccc32)CC1]

To an oven-dried flask DCM (0.5 mL) was added and cooled to 0° C. under N₂. Et₂Zn in DCM (1 M, 0.8 mL, 0.8 mmol) was added, and then TFA (62 μL, 0.8 mmol) in DCM (0.5 mL) was added to the mixture and stirred at 0° C. for 20 min. CH₂I₂ (65 μL, 0.8 mmol) in DCM (0.5 mL) was added to the mixture and stirred at 0° C. for 20 min. Example 73A (147 mg, 0.4 mmol) was dissolved in DCM (1 mL), added to the mixture and stirred at rt until no further conversion was observed. The reaction was quenched with water and extracted with DCM. The combined organic layers were dried over Na₂SO₄, filtered and the filtrate was concentrated under reduced pressure. The crude product was purified via flash chromatography using heptane and EtOAc as eluents to give Example 73B. ¹H NMR (500 MHz, DMSO-d₆) δ ppm: 7.32 (d, 1H), 7.29 (t, 1H), 7.25 (d, 1H), 7.2 (td, 1H), 7.14 (td, 1H), 7.13 (dm, 1H), 7.12 (m, 1H), 7.05 (dm, 1H), 7 (d, 1H), 6.79 (d, 1H), 3.74 (s, 3H), 2.84 (s, 3H), 2.32/1.89 (d+t, 4H), 1.77/1.27 (t+d, 4H). HRMS calculated for C₂₃H₂₄ClNO₂: 381.1496; found 382.1567 (M+H).

### Example 73 (1r,4r)-4-[(3-chlorophenyl)(methyl)amino]spiro[cyclohexane-1,1′-indene]-4-carboxylic acid

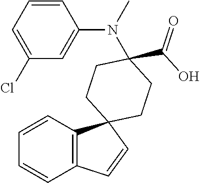 [SMILES: CN(c1cccc(Cl)c1)[C@]1(C(=O)O)CC[C@]2(C=Cc3ccccc32)CC1]

Using General procedure 33a and Example 73B as the appropriate ester Example 73 was obtained. ¹H NMR (500 MHz, DMSO-d₆) δ ppm: 12.88 (br s, 1H), 7.35-7 (m, 8H), 6.95 (d, 1H), 6.77 (d, 1H), 2.91 (s, 3H), 2.32/1.85 (m, 4H), 1.85/1.3 (m, 4H). HRMS calculated for C₂₂H₂₂NO₂Cl: 367.1339; found: 368.1414 (M+H).

### Example 74 (1r,4r)-2′-bromo-4-(3-chloroanilino)spiro[cyclohexane-1,1′-indene]-4-carboxylic acid

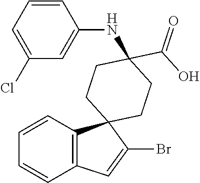 [SMILES: O=C(O)[C@]1(Nc2cccc(Cl)c2)CC[C@@]2(CC1)C(Br)=Cc1ccccc12]

Preparation 6b was purified via prep RP-HPLC using 25 mM aq. NH₄HCO₃ solution and MeCN as eluents resulting Example 74. ¹H NMR (500 MHz, DMSO-d₆) δ ppm: 7.7 (d, 1H), 7.35 (d, 1H), 7.27 (t, 1H), 7.21 (t, 1H), 7.06 (t, 1H), 7.03 (s, 1H), 6.74 (m, 1H), 6.65 (dd, 1H), 6.54 (dd, 1H), 2.62/2.07 (m+m, 4H), 2.03/1.33 (m+m, 4H). HRMS calculated for C₂₁H₁₉BrClNO₂: 431.0288; found 432.0360 (M+H).

### Example 75 (1s,4s)-2′-bromo-4-(3-chloroanilino)spiro[cyclohexane-1,1′-indene]-4-carboxylic acid

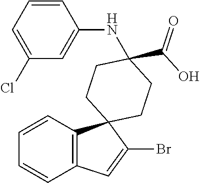 [SMILES: O=C(O)[C@]1(Nc2cccc(Cl)c2)CC[C@]2(CC1)C(Br)=Cc1ccccc12]

Preparation 6a was purified via prep RP-HPLC using 25 mM aq. NH₄HCO₃ solution and MeCN as eluents resulting Example 75. ¹H NMR (500 MHz, DMSO-d₆) δ ppm: 7.77 (d, 1H), 7.35 (dd, 1H), 7.28 (t, 1H), 7.21 (td, 1H), 7.01 (t, 1H), 7.01 (s, 1H), 6.62 (t, 1H), 6.56 (dd, 1H), 6.46 (dd, 1H), 2.4/2.17 (t+d, 4H), 2.16/0.92 (t+d, 4H). HRMS calculated for C₂₁H₁₉BrClNO₂: 431.0288; found 432.0358 (M+H).

### Example 76

### Example 76A 2″-iododispiro[[1,3]dioxolane-2,1′-cyclohexane-4′,1″-indene]

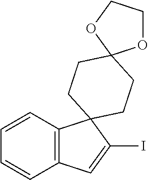 [SMILES: IC1=Cc2ccccc2C12CCC1(CC2)OCCO1]

Preparation 4aA (1.61 g, 5.0 mmol) was dissolved in dry THE (20 mL) and cooled to −78° C. under N₂. nBuLi in hexanes (2.5M, 2.2 mL, 5.5 mmol) was added to the mixture dropwise and stirred at −78° C. for 15 min. I₂ (1.52 g, 6.0 mmol) was added to the mixture, stirred at −78° C. for 20 min, then allowed to warm to rt and stirred at rt until no further conversion was observed. Sat. aq. NH₄Cl solution and sat. aq. Na₂S₂O₃ solution were added to the mixture and extracted with THF. The combined organic layers were dried over Na₂SO₄, filtered and the filtrate was concentrated under reduced pressure. The crude intermediate was purified via flash chromatography using heptane and EtOAc as eluents to obtain Example 76A. ¹H NMR (500 MHz, DMSO-d₆) δ ppm: 7.71 (d, 1H), 7.34 (dd, 1H), 7.27 (td, 1H), 7.24 (s, 1H), 7.16 (td, 1H), 3.96 (m, 4H), 2.14/1.86 (td+d, 4H), 2.05/1.05 (td+d, 4H). HRMS calculated for C₁₆H₁₇IO₂: 368.0273; found 369.0342 (M+H).

### Example 76B 2′-iodospiro[cyclohexane-4,1′-indene]-1-one

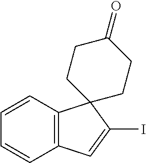 [SMILES: O=C1CCC2(CC1)C(I)=Cc1ccccc12]

Using General procedure 9 and Example 76A as the appropriate ketal Example 76B was obtained. ¹H NMR (500 MHz, DMSO-d₆) δ ppm: 7.96 (d, 1H), 7.38 (d, 1H), 7.31 (s, 1H), 7.31 (t, 1H), 7.18 (t, 1H), 2.98/2.47 (m, 4H), 2.14/1.44 (m, 4H). HRMS calculated for C₁₄H₁₃IO: 324.0011; found 342.0344 (M+NH₄).

### Example 76C (1s,4s)-4-(3-chloroanilino)-2′-iodospiro[cyclohexane-1,1′-indene]-4-carbonitrile

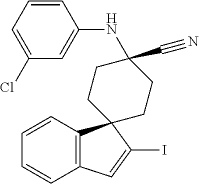 [SMILES: N#C[C@]1(Nc2cccc(Cl)c2)CC[C@]2(CC1)C(I)=Cc1ccccc12]

Using General procedure 11 and Example 76B as the appropriate ketone and 3-chloroaniline as the appropriate aniline a mixture of diastereoisomers was obtained. The diastereoisomers were separated via flash chromatography using heptane and EtOAc as eluents. The diastereoisomer eluting earlier was collected as Example 76C. ¹H NMR (500 MHz, DMSO-d₆) δ ppm: 7.86 (dm, 1H), 7.34 (dm, 1H), 7.29 (m, 1H), 7.25 (s, 1H), 7.25 (t, 1H), 7.21 (m, 1H), 6.94 (t, 1H), 6.9 (dm, 1H), 6.8 (dm, 1H), 6.61 (s, 1H), 2.6/2.51 (m+m, 4H), 2.06/1.06 (m+m, 4H). HRMS calculated for C₂₁H₁₈ClIN₂: 460.0203; found 461.0272 (M+H).

### Example 76D (1s,4s)-4-(3-chloroanilino)-2′-iodospiro[cyclohexane-1,1′-indene]-4-carboxamide

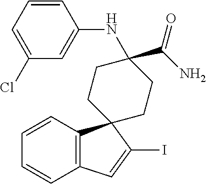 [SMILES: NC(=O)[C@]1(Nc2cccc(Cl)c2)CC[C@]2(CC1)C(I)=Cc1ccccc12]

Using General procedure 12b and Example 76C as the appropriate nitrile Example 76D was obtained. ¹H NMR (500 MHz, DMSO-d₆) δ ppm: 7.8 (dm, 1H), 7.34 (dm, 1H), 7.29/7.25 (br s+br s, 2H), 7.28 (m, 1H), 7.22 (s, 1H), 7.19 (m, 1H), 7.12 (t, 1H), 6.7 (t, 1H), 6.63 (dm, 1H), 6.61 (dm, 1H), 6.22 (s, 1H), 2.5/2.11 (m+m, 4H), 2.04/0.85 (m+m, 4H). HRMS calculated for C₂₁H₂₀ClIN₂O: 478.0309; found 479.0381 (M+H).

### Example 76 (1s,4s)-4-(3-chloroanilino)-2′-iodospiro[cyclohexane-1,1′-indene]-4-carboxylic acid

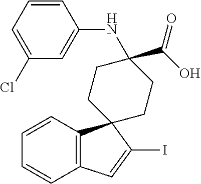 [SMILES: O=C(O)[C@]1(Nc2cccc(Cl)c2)CC[C@]2(CC1)C(I)=Cc1ccccc12]

Using General procedure 13 and Example 76D as the appropriate amide Example 76 was obtained. ¹H NMR (500 MHz, DMSO-d₆) δ ppm: 7.8 (dm, 1H), 7.33 (dm, 1H), 7.26 (m, 1H), 7.2 (s, 1H), 7.17 (m, 1H), 7.01 (t, 1H), 6.62 (t, 1H), 6.56 (dm, 1H), 6.47 (dm, 1H), 6.21 (br s, 1H), 2.42/2.19 (m+m, 4H), 2.07/0.81 (m+m, 4H). HRMS calculated for C₂₁H₁₉ClINO₂: 479.0149; found 480.0245 (M+H).

### Example 77

### Example 77A (1s,4s)-4-(3-bromoanilino)-2′-iodospiro[cyclohexane-1,1′-indene]-4-carbonitrile

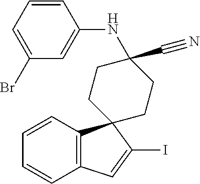 [SMILES: N#C[C@]1(Nc2cccc(Br)c2)CC[C@]2(CC1)C(I)=Cc1ccccc12]

Using General procedure 11 and Example 76B as the appropriate ketone and 3-bromoaniline as the appropriate aniline a mixture of diastereoisomers was obtained. The diastereoisomers were separated via flash chromatography using heptane and EtOAc as eluents. The diastereoisomer eluting earlier was collected as Example 77A. ¹H NMR (500 MHz, DMSO-d₆) δ ppm: 7.85 (dm, 1H), 7.34 (dm, 1H), 7.29 (m, 1H), 7.25 (s, 1H), 7.21 (m, 1H), 7.19 (t, 1H), 7.09 (t, 1H), 6.94 (dm, 1H), 6.93 (dm, 1H), 6.59 (s, 1H), 2.59/2.5 (m+m, 4H), 2.06/1.05 (m+m, 4H). HRMS calculated for C₂₁H₁₈BrIN₂: 503.9698; found 504.9762 (M+H).

### Example 77B (1s,4s)-4-(3-bromoanilino)-2′-iodospiro[cyclohexane-1,1′-indene]-4-carboxamide

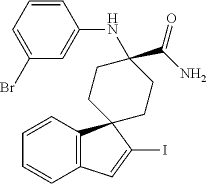 [SMILES: NC(=O)[C@]1(Nc2cccc(Br)c2)CC[C@]2(CC1)C(I)=Cc1ccccc12]

Using General procedure 12b and Example 77A as the appropriate nitrile Example 77B was obtained. ¹H NMR (500 MHz, DMSO-d₆) δ ppm: 7.79 (dm, 1H), 7.34 (dm, 1H), 7.3/7.26 (br s+br s, 2H), 7.27 (m, 1H), 7.22 (s, 1H), 7.19 (m, 1H), 7.06 (t, 1H), 6.86 (t, 1H), 6.76 (dm, 1H), 6.64 (dm, 1H), 6.21 (s, 1H), 2.5/2.1 (m+m, 4H), 2.04/0.84 (m+m, 4H). HRMS calculated for C₂₁H₂₀BrIN₂O: 521.9803; found 522.9887 (M+H).

### Example 77 (1s,4s)-4-(3-bromoanilino)-2′-iodospiro[cyclohexane-1,1′-indene]-4-carboxylic acid

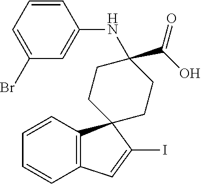 [SMILES: O=C(O)[C@]1(Nc2cccc(Br)c2)CC[C@]2(CC1)C(I)=Cc1ccccc12]

Using General procedure 13 and Example 77B as the appropriate amide Example 77 was obtained. ¹H NMR (500 MHz, DMSO-d₆) δ ppm: 7.79 (dm, 1H), 7.33 (dm, 1H), 7.27 (m, 1H), 7.21 (s, 1H), 7.18 (m, 1H), 6.97 (t, 1H), 6.78 (t, 1H), 6.62 (dm, 1H), 6.59 (dm, 1H), 6.22 (br s, 1H), 2.42/2.18 (m+m, 4H), 2.07/0.82 (m+m, 4H). HRMS calculated for C₂₁H₁₉BrINO₂: 522.9644; found 523.9726 (M+H).

### Example 78

### Example 78A (1s,4s)-2′-bromo-4-(3-bromoanilino)spiro[cyclohexane-1,1′-indene]-4-carbonitrile

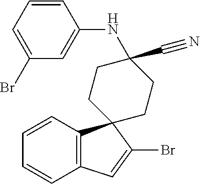 [SMILES: N#C[C@]1(Nc2cccc(Br)c2)CC[C@]2(CC1)C(Br)=Cc1ccccc12]

Using General procedure 11 and Preparation 4a as the appropriate ketone and 3-bromoaniline as the appropriate aniline a mixture of diastereoisomers was obtained. The diastereoisomers were separated via flash chromatography using heptane and EtOAc as eluents. The diastereoisomer eluting earlier was collected as Example 78A. ¹H NMR (500 MHz, DMSO-d₆) δ ppm: 7.77 (dm, 1H), 7.36 (dm, 1H), 7.3 (m, 1H), 7.25 (m, 1H), 7.18 (t, 1H), 7.09 (t, 1H), 7.06 (s, 1H), 6.94 (dm, 1H), 6.92 (dm, 1H), 6.58 (s, 1H), 2.57/2.49 (m+m, 4H), 2.1/1.26 (m+m, 4H). HRMS calculated for C₂₁H₁₈Br₂N₂: 455.9837; found 456.9921 (M+H).

### Example 78B (1s,4s)-2′-bromo-4-(3-bromoanilino)spiro[cyclohexane-1,1′-indene]-4-carboxamide

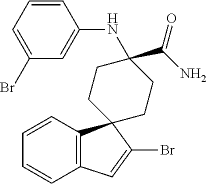 [SMILES: NC(=O)[C@]1(Nc2cccc(Br)c2)CC[C@]2(CC1)C(Br)=Cc1ccccc12]

Using General procedure 12b and Example 78A as the appropriate nitrile Example 78B was obtained. ¹H NMR (500 MHz, DMSO-d₆) δ ppm: 7.76 (dm, 1H), 7.36 (dm, 1H), 7.34/7.25 (d+d, 2H), 7.29 (m, 1H), 7.22 (m, 1H), 7.06 (t, 1H), 7.02 (s, 1H), 6.86 (t, 1H), 6.75 (dm, 1H), 6.63 (dm, 1H), 6.22 (s, 1H), 2.46/2.1 (m+m, 4H), 2.13/0.94 (m+m, 4H). HRMS calculated for C₂₁H₂₀Br₂N₂O: 473.9942; found 475.0015 (M+H).

### Example 78 (1s,4s)-2′-bromo-4-(3-bromoanilino)spiro[cyclohexane-1,1′-indene]-4-carboxylic acid

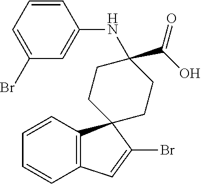 [SMILES: O=C(O)[C@]1(Nc2cccc(Br)c2)CC[C@]2(CC1)C(Br)=Cc1ccccc12]

Using General procedure 13 and Example 78B as the appropriate amide Example 78 was obtained. ¹H NMR (500 MHz, DMSO-d₆) δ ppm: 7.72 (dm, 1H), 7.37 (dd, 1H), 7.29 (td, 1H), 7.23 (td, 1H), 7.03 (s, 1H), 7.02 (t, 1H), 6.78 (t, 1H), 6.69 (dm, 1H), 6.59 (dm, 1H), 6.39 (br, 1H), 2.39/2.24 (tm+d, 4H), 2.19/0.97 (tm+d, 4H). HRMS calculated for C₂₁H₁₉NO₂Br₂: 474.9782; found: 475.9851 (M+H).

### Example 79

### Example 79A (1s,4s)-2′-bromo-4-[(5-bromopyridin-3-yl)amino]spiro[cyclohexane-1,1′-indene]-4-carbonitrile

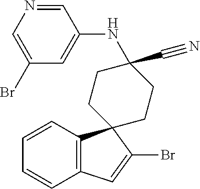 [SMILES: N#C[C@]1(Nc2cncc(Br)c2)CC[C@]2(CC1)C(Br)=Cc1ccccc12]

Using General procedure 11 and Preparation 4a as the appropriate ketone and 5-bromopyridin-3-amine as the appropriate pyridin-amine a mixture of diastereoisomers was obtained. The diastereoisomers were separated via flash chromatography using heptane and EtOAc as eluents. The diastereoisomer eluting earlier was collected as Example 79A. ¹H NMR (500 MHz, DMSO-d₆) δ ppm: 8.27 (d, 1H), 8.09 (d, 1H), 7.78 (d, 1H), 7.45 (t, 1H), 7.36 (dd, 1H), 7.31 (td, 1H), 7.25 (td, 1H), 7.07 (s, 1H), 6.91 (s, 1H), 2.57/2.53 (tm+d, 4H), 2.09/1.32 (tm+d, 4H). HRMS calculated for C₂₀H₁₇Br₂N₃: 456.9789; found 457.9864 (M+H).

### Example 79B (1s,4s)-2′-bromo-4-[(5-bromopyridin-3-yl)amino]spiro[cyclohexane-1,1′-indene]-4-carboxamide

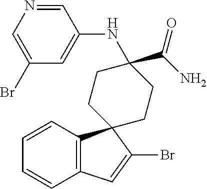 [SMILES: NC(=O)[C@]1(Nc2cncc(Br)c2)CC[C@]2(CC1)C(Br)=Cc1ccccc12]

Using General procedure 12a and Example 79A as the appropriate nitrile Example 79B was obtained. ¹H NMR (500 MHz, DMSO-d₆) δ ppm: 8.05 (d, 1H), 7.89 (d, 1H), 7.78-7.19 (m, 4H), 7.53/7.31 (s+s, 2H), 7.08 (t, 1H), 7.03 (s, 1H), 6.57 (s, 1H), 2.5/2.08 (m+m, 4H), 2.14/0.97 (m+m, 4H). HRMS calculated for C₂₀H₁₉Br₂N₃O: 474.9895; found 475.9964 (M+H).

### Example 79 (1s,4s)-2′-bromo-4-[(5-bromopyridin-3-yl)amino]spiro[cyclohexane-1,1′-indene]-4-carboxylic acid

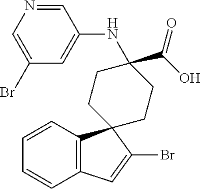 [SMILES: O=C(O)[C@]1(Nc2cncc(Br)c2)CC[C@]2(CC1)C(Br)=Cc1ccccc12]

Using General procedure 13 and Example 79B as the appropriate amide Example 79 was obtained. ¹H NMR (500 MHz, DMSO-d₆) δ ppm: 13.15 (br, 1H), 8.01 (d, 1H), 7.86 (d, 1H), 7.73 (d, 1H), 7.37 (dd, 1H), 7.3 (td, 1H), 7.24 (td, 1H), 7.04 (s, 1H), 7.04 (t, 1H), 6.69 (br s, 1H), 2.44/2.23 (td+d, 4H), 2.17/1 (td+d, 4H). HRMS calculated for C₂₀H₁₈N₂O₂Br₂: 475.9735; found: 476.9841 (M+H).

### Example 80

### Example 80A (1s,4s)-2′-bromo-4-[(6-bromopyridin-2-yl)amino]spiro[cyclohexane-1,1′-indene]-4-carbonitrile

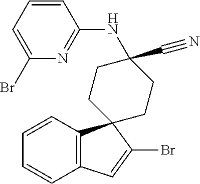 [SMILES: N#C[C@]1(Nc2cccc(Br)n2)CC[C@]2(CC1)C(Br)=Cc1ccccc12]

Using General procedure 11 and Preparation 4a as the appropriate ketone and 6-bromopyridin-2-amine as the appropriate pyridin-amine a mixture of diastereoisomers was obtained. The diastereoisomers were separated via flash chromatography using heptane and EtOAc as eluents. The diastereoisomer eluting earlier was collected as Example 80A. ¹H NMR (500 MHz, DMSO-d₆) δ ppm: 7.77 (dm, 1H), 7.58 (s, 1H), 7.46 (dd, 1H), 7.36 (dm, 1H), 7.3 (m, 1H), 7.25 (m, 1H), 7.07 (s, 1H), 6.9 (d, 1H), 6.77 (d, 1H), 2.8/2.49 (m+m, 4H), 2.08/1.27 (m+m, 4H). HRMS calculated for C₂₀H₁₇Br₂N₃: 456.9789; found 457.9851 (M+H).

### Example 80B (1s,4s)-2′-bromo-4-[(6-bromopyridin-2-yl)amino]spiro[cyclohexane-1,1′-indene]-4-carboxamide

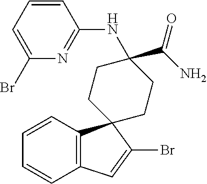 [SMILES: NC(=O)[C@]1(Nc2cccc(Br)n2)CC[C@]2(CC1)C(Br)=Cc1ccccc12]

Using General procedure 12a and Example 80A as the appropriate nitrile Example 80B was obtained. ¹H NMR (500 MHz, DMSO-d₆) δ ppm: 7.83-7.19 (m, 4H), 7.35 (t, 1H), 7.11 (s, 1H), 7.09/7 (s+s, 2H), 7.02 (s, 1H), 6.71 (dd, 1H), 6.71 (dd, 1H), 2.5-0.9 (m, 8H). HRMS calculated for C₂₀H₁₉Br₂N₃O: 474.9895; found 475.9972 (M+H).

### Example 80 (1s,4s)-2′-bromo-4-{[6-(2-methoxyethoxy)pyridin-2-yl]amino}spiro[cyclohexane-1,1′-indene]-4-carboxylic acid

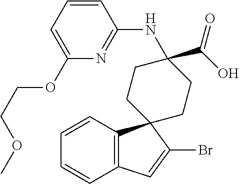 [SMILES: COCCOc1cccc(N[C@]2(C(=O)O)CC[C@@]3(CC2)C(Br)=Cc2ccccc23)n1]

Using General procedure 13 and Example 80B as the appropriate amide Example 80 was obtained. ¹H NMR (400 MHz, DMSO-d₆) δ ppm: 9.83 (br s, 1H), 7.73 (dm, 1H), 7.37 (dm, 1H), 7.3 (t, 1H), 7.29 (m, 1H), 7.22 (m, 1H), 7.03 (s, 1H), 6.99 (br s, 1H), 6.24 (d, 1H), 5.86 (d, 1H), 4.19 (m, 2H), 3.55 (m, 2H), 3.26 (s, 3H), 2.5-2.3 (m, 4H), 2.21-0.97 (m+m, 4H). HRMS calculated for C₂₃H₂₅N₂O₄Br: 472.0998; found: 473.1068 (M+H).

### Example 81

### Example 81A (1s,4s)-2′-bromo-4-[(2-bromopyridin-4-yl)amino]spiro[cyclohexane-1,1′-indene]-4-carbonitrile

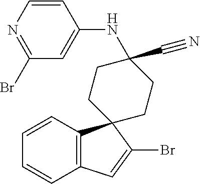 [SMILES: N#C[C@]1(Nc2ccnc(Br)c2)CC[C@]2(CC1)C(Br)=Cc1ccccc12]

Using General procedure 11 and Preparation 4a as the appropriate ketone and 2-bromopyridin-4-amine as the appropriate pyridin-amine a mixture of diastereoisomers was obtained. The diastereoisomers were separated via flash chromatography using heptane and EtOAc as eluents. The diastereoisomer eluting earlier was collected as Example 81A. ¹H NMR (500 MHz, DMSO-d₆) δ ppm: 8.02 (d, 1H), 7.77 (d, 1H), 7.52 (s, 1H), 7.36 (dd, 1H), 7.3 (td, 1H), 7.25 (td, 1H), 7.07 (s, 1H), 6.99 (d, 1H), 6.91 (dd, 1H), 2.58/2.52 (dm+td, 4H), 2.05/1.35 (td+d, 4H). HRMS calculated for C₂₀H₁₇Br₂N₃: 456.9789; found 457.9855 (M+H).

### Example 81B (1s,4s)-2′-bromo-4-[(2-bromopyridin-4-yl)amino]spiro[cyclohexane-1,1′-indene]-4-carboxamide

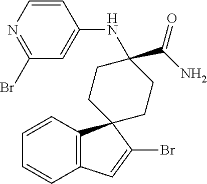 [SMILES: NC(=O)[C@]1(Nc2ccnc(Br)c2)CC[C@]2(CC1)C(Br)=Cc1ccccc12]

Using General procedure 12a and Example 81A as the appropriate nitrile Example 81B was obtained. ¹H NMR (500 MHz, DMSO-d₆) δ ppm: 7.86 (d, 1H), 7.72 (dm, 1H), 7.48/7.31 (s+s, 2H), 7.36 (dm, 1H), 7.3 (m, 1H), 7.23 (m, 1H), 7.18 (s, 1H), 7.04 (s, 1H), 6.73 (br s, 1H), 6.59 (br d, 1H), 2.54-0.93 (m, 8H). HRMS calculated for C₂₀H₁₉Br₂N₃O: 474.9895; found 475.9971 (M+H).

### Example 81 (1s,4s)-2′-bromo-4-{[2-(2-methoxyethoxy)pyridin-4-yl]amino}spiro[cyclohexane-1,1′-indene]-4-carboxylic acid

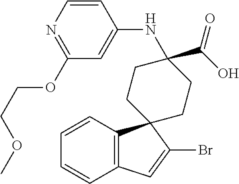 [SMILES: COCCOc1cc(N[C@]2(C(=O)O)CC[C@@]3(CC2)C(Br)=Cc2ccccc23)ccn1]

Using General procedure 13 and Example 81B as the appropriate amide Example 81 was obtained. ¹H NMR (500 MHz, DMSO-d₆) δ ppm: 7.71 (d, 1H), 7.65 (d, 1H), 7.37 (dd, 1H), 7.29 (td, 1H), 7.23 (td, 1H), 7.04 (s, 1H), 6.86 (br s, 1H), 6.35 (dd, 1H), 5.72 (d, 1H), 4.24 (m, 2H), 3.58 (m, 2H), 3.27 (s, 3H), 2.39/2.25 (td+d, 4H), 2.17/1 (td+d, 4H). HRMS calculated for C₂₃H₂₅N₂O₄Br: 472.0998; found: 473.1070 (M+H).

### Example 82 (1s,4s)-2′-bromo-4-(3-cyanoanilino)spiro[cyclohexane-1,1′-indene]-4-carboxylic acid

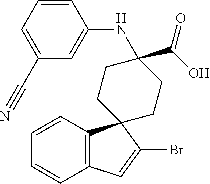 [SMILES: N#Cc1cccc(N[C@]2(C(=O)O)CC[C@@]3(CC2)C(Br)=Cc2ccccc23)c1]

A solution of Preparation 5a (120 mg, 0.37 mmol, 1 eq) in DMF (2 mL) was treated as described in General procedure 16 using 3-iodobenzonitrile (102 mg, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase was washed with a 1:1 mixture of 0.1 M aq. HCl solution and brine, dried (PTFE phase separator) and concentrated in vacuo. Purification by automated flash chromatography (Combiflash Rf, 12 g RediSep™ silica cartridge) eluting with a gradient of 0-80% EtOAc in heptane afforded Example 82 as a yellow glass (65.3 mg, 0.15 mmol, 41%). LRMS calculated for C₂₂H₁₉N₂O₂Br: 422; found: 423 (M+H). ¹H NMR (400 MHz, DMSO-d₆) δ 12.96 (s, 1H), 7.73 (d, J=7.4 Hz, 1H), 7.38 (dd, J=7.3, 1.3 Hz, 1H), 7.34-7.21 (m, 3H), 7.05 (s, 1H), 7.00-6.97 (m, 1H), 6.96-6.92 (m, 1H), 6.87 (dd, J=2.5, 1.5 Hz, 1H), 6.64 (s, 1H), 2.43 (td, J=14.0, 3.8 Hz, 2H), 2.32-2.15 (m, 4H), 1.05-1.97 (m, 2H).

### Example 83 (1s,4s)-2′-bromo-4-[3-(difluoromethyl)anilino]spiro[cyclohexane-1,1′-indene]-4-carboxylic acid

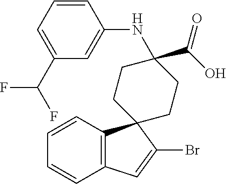 [SMILES: O=C(O)[C@]1(Nc2cccc(C(F)F)c2)CC[C@]2(CC1)C(Br)=Cc1ccccc12]

A solution of Preparation 5a (120 mg, 0.37 mmol, 1 eq) in DMF (2 mL) was treated as described in General procedure 16 using 1-(difluoromethyl)-3-iodobenzene (114 mg, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase was washed with a 1:1 mixture of 0.1 M aq. HCl solution and brine, dried (PTFE phase separator) and concentrated in vacuo. Purification by automated flash chromatography (Combiflash Rf, 12 g, RediSep™ silica cartridge) eluting with a gradient of 0-70% EtOAc in heptane was followed by trituration with Et₂O. The solid was collected by filtration and dried in vacuo at 45° C. to afford Example 83 as a white powder (64.4 mg, 0.14 mmol, 39%). LRMS calculated for C₂₂H₂₀NO₂F₂Br: 447; found: 448 (M+H). ¹H NMR (400 MHz, DMSO-d₆) δ 12.79 (br s, 1H), 7.73 (d, J=7.5 Hz, 1H), 7.38 (dd, J=7.3, 1.4 Hz, 1H), 7.31 (td, J=7.5, 1.1 Hz, 1H), 7.28-7.18 (m, 2H), 7.04 (s, 1H), 6.90 (t, J=56.3 Hz, 1H), 6.82 (app s, 1H), 6.77-6.72 (m, 2H), 6.42 (br s, 1H), 2.48-2.37 (m, 2H), 2.33-2.17 (m, 4H), 1.03-0.95 (m, 2H).

### Example 84 (1s,4s)-2′-bromo-4-(3-hydroxyanilino)spiro[cyclohexane-1,1′-indene]-4-carboxylic acid

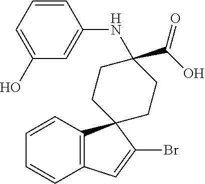 [SMILES: O=C(O)[C@]1(Nc2cccc(O)c2)CC[C@]2(CC1)C(Br)=Cc1ccccc12]

A solution of Preparation 5a (120 mg, 0.37 mmol, 1 eq) in DMF (2 mL) was treated as described in General procedure 16 using 3-iodophenol (98 mg, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase was washed with a 1:1 mixture of 0.1 M aq. HCl solution and brine, dried (PTFE phase separator) and concentrated in vacuo. Purification by automated flash chromatography (Combiflash Rf, 12 g, RediSep™ silica cartridge) eluting with a gradient of 0-100% EtOAc in heptane, followed by preparative HPLC in a gradient of 10-100% MeCN in water at pH 4 afforded Example 84 as a white powder (41 mg, 0.1 mmol, 27%). LRMS calculated for C₂₁H₂₀NO₃Br: 413; found: 414 (M+H). ¹H NMR (400 MHz, DMSO-d₆) δ ppm: 8.94 (br s, 1H), 7.73 (d, J=7.5 Hz, 1H), 7.37 (dd, J=7.3, 1.3 Hz, 1H), 7.30 (td, J=7.5, 0.7 Hz, 1H), 7.23 (td, J=7.5, 1.2 Hz, 1H), 7.03 (s, 1H), 6.83 (t, J=7.9 Hz, 1H), 6.12-6.06 (m, 2H), 5.99 (dd, J=7.5, 2.0 Hz, 1H), 2.42-2.16 (m, 6H), 0.99-0.90 (m, 2H).

### Example 85 (1s,4s)-2′-bromo-4-[3-(trifluoromethyl)anilino]spiro[cyclohexane-1,1′-indene]-4-carboxylic acid

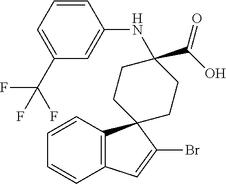 [SMILES: O=C(O)[C@]1(Nc2cccc(C(F)(F)F)c2)CC[C@]2(CC1)C(Br)=Cc1ccccc12]

A solution of Preparation 5a (120 mg, 0.37 mmol, 1 eq) in DMF (2 mL) was treated as described in General procedure 16 using 1-iodo-3-(trifluoromethyl)benzene (122 mg, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase was washed with a 1:1 mixture of 0.1 M aq. HCl solution and brine, dried (PTFE phase separator) and concentrated in vacuo. Purification by reverse phase automated flash chromatography (Combiflash Rf, C18 13 g, RediSep™ column) eluting with a gradient of 25-100% MeCN in water afforded Example 85 as a white powder (51.9 mg, 0.11 mmol, 30%). LRMS calculated for C₂₂H₁₉NO₂F₃Br: 465; found: 466 (M+H). ¹H NMR (400 MHz, DMSO-d₆) δ ppm: 12.92 (br s, 1H), 7.73 (d, J=7.4 Hz, 1H), 7.38 (dd, J=7.3, 1.3 Hz, 1H), 7.33-7.28 (m, 2H), 7.25 (td, J=7.5, 1.5 Hz, 1H), 7.05 (s, 1H), 6.93 (app s, 1H), 6.89-6.82 (m, 2H), 6.61 (br s, 1H), 2.44 (td, J=15.1, 14.5, 4.0 Hz, 2H), 2.32-2.17 (m, 4H), 1.05-0.95 (m, 2H).

### Example 86 (1s,4s)-2′-bromo-4-(3-methoxyanilino)spiro[cyclohexane-1,1′-indene]-4-carboxylic acid

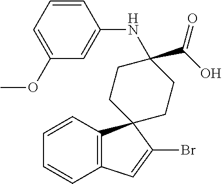 [SMILES: COc1cccc(N[C@]2(C(=O)O)CC[C@@]3(CC2)C(Br)=Cc2ccccc23)c1]

A solution of Preparation 5a (120 mg, 0.37 mmol, 1 eq) in DMF (2 mL) was treated as described in General procedure 16 using 3-iodoanisole (53 μL, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase washed with a 1:1 mixture of 0.1 M aq. HCl solution and brine, dried (PTFE phase separator) and concentrated in vacuo. Purification by reverse phase automated flash chromatography (Combiflash Rf, C18 13 g, RediSep™ column) eluting with a gradient of 25-100% MeCN in water was followed by automated flash chromatography (Combiflash Rf, 4 g RediSep™ silica cartridge) eluting with a gradient of 0-75% EtOAc in heptane. The resultant solids were triturated with heptane, collected by filtration and dried in vacuo to afford Example 86 as a beige powder (17.7 mg, 0.04 mmol, 11%). LRMS calculated for C₂₂H₂₂NO₃Br: 427; found: 428 (M+H). ¹H NMR (400 MHz, DMSO-d₆) δ ppm: 7.72 (d, J=7.5 Hz, 1H), 7.37 (dd, J=7.4, 1.3 Hz, 1H), 7.30 (td, J=7.5, 1.0 Hz, 1H), 7.23 (td, J=7.5, 1.4 Hz, 1H), 7.04 (s, 1H), 6.97 (t, J=8.1 Hz, 1H), 6.24 (dd, J=8.4, 2.1 Hz, 1H), 6.20-6.14 (m, 2H), 3.67 (s, 3H), 2.43-2.17 (m, 6H), 1.00-0.92 (m, 2H).

### Example 87 (1s,4s)-2′-bromo-4-[(pyridin-3-yl)amino]spiro[cyclohexane-1,1′-indene]-4-carboxylic acid

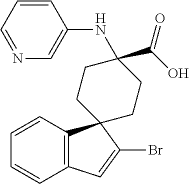 [SMILES: O=C(O)[C@]1(Nc2cccnc2)CC[C@]2(CC1)C(Br)=Cc1ccccc12]

A solution of Preparation 5a (120 mg, 0.37 mmol, 1 eq) in DMF (3 mL) was treated as described in General procedure 16 using 3-iodopyridine (92 mg, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase washed with a 1:1 mixture of 0.1 M aq. HCl solution and brine, dried (PTFE phase separator) and concentrated in vacuo. Purification by reverse phase automated flash chromatography (Combiflash Rf, C18 13 g, RediSep™ column) eluting with a gradient of 20-100% MeCN in water afforded Example 87 as a yellow powder (31.8 mg, 0.08 mmol, 21%). LRMS calculated for C₂₀H₁₉N₂O₂Br: 398; found: 399 (M+H). ¹H NMR (400 MHz, DMSO-d₆) δ ppm: 12.88 (br s, 1H), 8.06 (br s, 1H), 7.81 (br s, 1H), 7.74 (d, J=7.5 Hz, 1H), 7.38 (dd, J=7.3, 1.3 Hz, 1H), 7.31 (td, J=7.5, 0.7 Hz, 1H), 7.25 (td, J=7.5, 1.2 Hz, 1H), 7.12 (br s, 1H), 7.05 (s, 1H), 6.92 (d, J=8.3 Hz, 1H), 6.35 (br s, 1H), 2.48-2.38 (m, 2H), 2.32-2.17 (m, 4H), 1.03-0.95 (m, 2H).

### Example 88 (1s,4s)-2′-bromo-4-(3-methylanilino)spiro[cyclohexane-1,1′-indene]-4-carboxylic acid

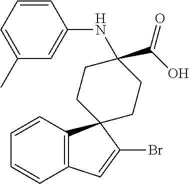 [SMILES: Cc1cccc(N[C@]2(C(=O)O)CC[C@@]3(CC2)C(Br)=Cc2ccccc23)c1]

A solution of Preparation 5a (120 mg, 0.37 mmol, 1 eq) in DMF (3 mL) was treated as described in General procedure 16 using 3-iodotoluene (57 μL, 0.45 mmol, 1.2 eq) and 2-(2-methylpropanoyl)cyclohexan-1-one (12 μL, 0.07 mmol, 0.2 eq) and the reaction was heated at 120° C. for 1 h under microwave irradiation. The reaction was diluted with DCM and the organic phase was washed with a 1:1 mixture of 0.1 M aq. HCl solution and brine, dried (PTFE phase separator) and concentrated in vacuo. Purification by automated flash chromatography (Combiflash Rf, 12 g RediSep™ silica cartridge) eluting with a gradient of 0-72% EtOAc in heptane was followed by trituration with minimal Et₂O. The solids were collected by filtration and dried in vacuo to afford Example 88 as a white powder (31.1 mg, 0.08 mmol, 20%). LRMS calculated for C₂₂H₂₂NO₂Br: 411; found: 412 (M+H). ¹H NMR (400 MHz, DMSO-d₆) δ ppm: 7.72 (d, J=7.5 Hz, 1H), 7.37 (dd, J=7.4, 1.3 Hz, 1H), 7.30 (td, J=7.4, 1.0 Hz, 1H), 7.24 (td, J=7.5, 1.5 Hz, 1H), 7.04 (s, 1H), 6.96 (t, J=7.7 Hz, 1H), 6.46 (app s, 1H), 6.44-6.38 (m, 2H), 2.38 (td, J=14.0, 3.6 Hz, 2H), 2.33-2.17 (m, 4H), 2.19 (s, 3H), 1.00-0.92 (m, 2H).

### Example 89

### Example 89A 6′-methoxyspiro[cyclopentane-1,2′-inden]-1′(3′H)-one

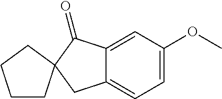 [SMILES: COc1ccc2c(c1)C(=O)C1(CCCC1)C2]

To a solution of 6-methoxy-1-indanone (2.08 g, 12.8 mmol, 1 eq) and 1,4-diiodobutane (2.11 mL, 16 mmol, 1.25 eq) in DMF (35 mL) was added NaH (60% dispersion; 1.28 g, 32.1 mmol, 2.5 eq) portionwise. The mixture was allowed to stir at rt for 30 min and then warmed to 50° C. for 18 h. The mixture was quenched with water and extracted with EtOAc and the combined organic extracts were washed successively with water and brine, dried (MgSO₄) and concentrated in vacuo. Purification by automated flash column chromatography (CombiFlash Rf, 80 g RediSep™ silica cartridge) eluting with a gradient of 0-100% EtOAc in heptane afforded Example 89A as a colourless oil (1.76 g, 8.14 mmol, 64%). LRMS calculated for C₁₄H₁₆O₂: 216; found: 217 (M+H). ¹H NMR (400 MHz, DMSO-d₆) δ ppm: 7.45 (d, J=8.4 Hz, 1H), 7.28 (dd, J=8.4, 2.6 Hz, 1H), 7.11 (d, J=2.5 Hz, 1H), 3.81 (s, 3H), 2.97 (s, 2H), 1.89-1.68 (m, 6H), 1.66-1.54 (m, 2H).

### Example 89B 6′-methoxy-1′-(methoxymethylidene)-1′,3′-dihydrospiro[cyclopentane-1,2′-indene]

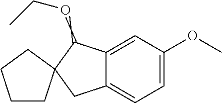

(Methoxymethyl)triphenylphosphonium chloride (6.42 g, 18.7 mmol, 2.3 eq) was added to a suspension of KOtBu (2.1 g, 18.7 mmol, 2.3 eq) in 1,4-dioxane (60 mL) and then stirred at rt for 2 h. A solution of Example 89A (1.76 g, 8.14 mmol, 1 eq) in 1,4-dioxane (25 mL) was added and the mixture was stirred at rt for 18 h. The mixture was quenched with water (50 mL) and extracted with EtOAc and the combined organic extracts were washed successively with water and brine, dried (MgSO₄) and concentrated in vacuo. Purification by automated flash column chromatography (CombiFlash Rf, 40 g RediSep™ silica cartridge) eluting with a gradient of 0-70% EtOAc in heptane afforded a 1.3:1 mixture of isomers, Example 89B as a colourless oil (1.71 g, 7 mmol, 86%). LRMS calculated for C₁₆H₂₀O₂: 244; found: 245 (M+H). ¹H NMR (400 MHz, DMSO-d₆) δ ppm: 7.24/6.89 (d, 1H), 7.06/6.99 (d, 1H), 6.88/6.28 (s, 1H), 6.69/6.62 (dd, 1H), 3.72/3.72 (s, 3H), 3.71/3.67 (s, 3H), 2.71/2.68 (s, 2H), 2.20-2.08 (m, 1H), 1.81-1.53 (m, 6H), 1.50-1.42 (m, 1H).

### Example 89C 6′-methoxy-3′H-dispiro[cyclohex-2-ene-1,1′-indene-2′,1″-cyclopentan]-4-one

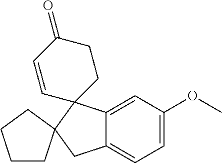 [SMILES: COc1ccc2c(c1)C1(C=CC(=O)CC1)C1(CCCC1)C2]

To a solution of Example 89B (1.71 g, 7 mmol, 1 eq) in toluene (35 mL) was added 3-buten-2-one (0.64 mL, 7.7 mmol, 1.1 eq) and PTSA (0.07 mL, 0.7 mmol, 0.1 eq). The mixture was stirred at rt for 30 min, then heated at 85° C. for 3 h. The reaction was allowed to cool to rt then partitioned between EtOAc and sat. aq. NaHCO₃ solution, and the organic phase was washed with brine, dried (MgSO₄) and concentrated in vacuo. Purification by automated flash column chromatography (CombiFlash Rf, 80 g RediSep™ silica cartridge) eluting with a gradient of 30-100% DCM in heptane afforded Example 89C as a racemic white solid (1.06 g, 3.75 mmol, 54%). LRMS calculated for C₁₉H₂₂O₂: 282; found: 283 (M+H). ¹H NMR (400 MHz, DMSO-d₆) δ ppm: 7.16 (d, J=8.2 Hz, 1H), 6.94 (d, J=10.3 Hz, 1H), 6.76 (dd, J=8.2, 2.5 Hz, 1H), 6.68 (d, J=2.4 Hz, 1H), 6.04 (d, J=10.3 Hz, 1H), 3.71 (s, 3H), 2.82 (d, J=15.4 Hz, 1H), 2.75 (d, J=15.4 Hz, 1H), 2.55-2.37 (m, 2H), 2.27-2.17 (m, 1H), 2.08-1.98 (m, 1H), 1.73-1.50 (m, 7H), 1.40-1.32 (m, 1H).

### Example 89D 6′-methoxy-3′H-dispiro[cyclohexane-1,1′-indene-2′,1″-cyclopentan]-4-one

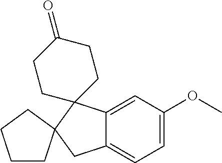 [SMILES: COc1ccc2c(c1)C1(CCC(=O)CC1)C1(CCCC1)C2]

To a solution of Example 89C (1.06 g, 3.75 mmol, 1 eq) in AcOH (200 mL) was added catalytic Pd/C under a N₂ atmosphere. The mixture was evacuated and backfilled with N₂, then evacuated and backfilled with H₂ and shaken for 18 h at rt under an atmosphere of H₂. The reaction was filtered through celite, eluted with DCM and evaporated under reduced pressure to afford Example 89D as a white solid (1.1 g, 3.87 mmol, quant.). LRMS calculated for C₁₉H₂₄O₂: 284; found: 285 (M+H). ¹H NMR (400 MHz, DMSO-d₆) δ ppm: 7.11 (d, J=8.1 Hz, 1H), 6.95 (d, J=2.4 Hz, 1H), 6.72 (dd, J=8.1, 2.4 Hz, 1H), 3.74 (s, 3H), 2.67 (s, 2H), 2.62-2.51 (m, 2H), 2.35-2.26 (m, 2H), 2.01-1.85 (m, 4H), 1.68-1.50 (m, 6H), 1.36-1.27 (m, 2H).

### Example 89 (1r,4r)-4-(3-chloroanilino)-6′-methoxy-3′H-dispiro[cyclohexane-1,1′-indene-2′,1″-cyclopentane]-4-carboxylic acid

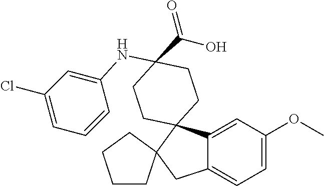 [SMILES: COc1ccc2c(c1)[C@]1(CC[C@@](Nc3cccc(Cl)c3)(C(=O)O)CC1)C1(CCCC1)C2]

A solution of Example 89D (204 mg, 0.72 mmol, 1 eq) in THE (14 mL) was treated as described in General procedure 10 using 3-chloroaniline (83 μL, 0.79 mmol, 1.1 eq) and the mixture was stirred at rt for 18 h. A mixture of diastereoisomers was obtained. The mixture was partitioned between DCM and water, the pH adjusted to 4 with 1 M aq. HCl solution and extracted with DCM. The organic phase was washed with brine, dried (PTFE phase separator) and concentrated in vacuo and purified by automated flash column chromatography (CombiFlash Rf, 40 g RediSep™ silica cartridge) eluting with a gradient of 0-40% EtOAc in heptane. The diastereoisomer eluting later was collected as Example 89 as an off-white solid (7.7 mg, 0.02 mmol, 2%). LRMS calculated for C₂₆H₃₀ClNO₃: 439; found: 440 (M+H). ¹H NMR (400 MHz, DMSO-d₆) δ ppm: 12.64 (br s, 1H), 7.12-7.03 (m, 2H), 7.02-6.96 (m, 1H), 6.72-6.67 (m, 1H), 6.60-6.47 (m, 3H), 6.19 (br s, 1H), 3.76-3.66 (m, 3H), 2.60 (s, 2H), 2.15-2.00 (m, 4H), 1.84-1.72 (m, 2H), 1.69-1.41 (m, 8H), 1.31-1.18 (m, 2H).

### Example 90 (1r,4r)-4-(3-chloroanilino)-2′-cyclopropylspiro[cyclohexane-1,1′-indene]-4-carboxylic acid

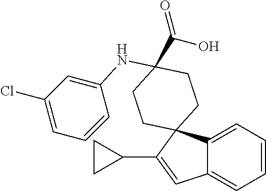 [SMILES: O=C(O)[C@]1(Nc2cccc(Cl)c2)CC[C@]2(CC1)C(C1CC1)=Cc1ccccc12]

A microwave vial was charged with Preparation 6c (0.152 g, 0.34 mmol), 2-cyclopropyl-4,4,5,5-tetramethyl-1,3,2-dioxaborolane (0.170 g, 1.01 mmol), Cs₂CO₃ (0.330 g, 1.01 mmol) and 1,4-dioxane (3 mL) and water (1 mL). The vial was purged with N₂, followed by the addition of Pd(PPh₃)₄ (0.039 g, 0.034 mmol). The mixture was heated at 120° C. for 30 min under microwave irradiation. To the crude mixture LiOH×H₂O (450 mg, 10.7 mmol) and water (2 mL) were added and the mixture was stirred at 65° C. until no further conversion was observed. The mixture was allowed to cool to rt. The pH was set to 3-4 with 2 M aq. HCl solution, and the mixture was extracted with DCM. The combined organic layers were dried over Na₂SO₄, filtered and the filtrate was concentrated under reduced pressure. The crude product was purified via prep RP-HPLC using 25 mM aq. NH₄HCO₃ solution and MeCN as eluents resulting Example 90. HRMS calculated for C₂₄H₂₄NO₂Cl: 393.1496; found: 394.1580 (M+H).

### Example 91 (1r,4r)-4-(3-chloroanilino)-2′-cyclopentyl-2′,3′-dihydrospiro[cyclohexane-1,1′-indene]-4-carboxylic acid, enantiomer 1

and

### Example 92 (1r,4r)-4-(3-chloroanilino)-2′-cyclopentyl-2′,3′-dihydrospiro[cyclohexane-1,1′-indene]-4-carboxylic acid, enantiomer 2

and

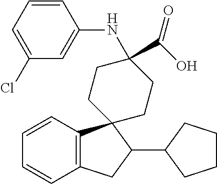

### Example 93 (1r,4r)-4-(3-chloroanilino)-2′-cyclopentylidene-2′,3′-dihydrospiro[cyclohexane-1,1′-indene]-4-carboxylic acid

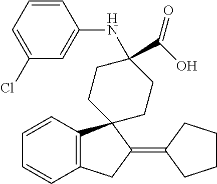 [SMILES: O=C(O)[C@]1(Nc2cccc(Cl)c2)CC[C@]2(CC1)C(=C1CCCC1)Cc1ccccc12]

Using General procedure 19 and Example 277 in AcOH a mixture of 3 compounds was obtained. It was purified and partly separated via prep RP-HPLC using 25 mM aq. NH₄HCO₃ solution and MeCN as eluents. The fractions containing the fully saturated enantiomers were separated by chiral chromatography. Column: AD, 50×500 mm, 20 μm, eluents: 15:85 EtOH/heptane+0.05% HCOOH. The enantiomer eluting earlier was collected as Example 91. HRMS calculated for C₂₆H₃₀NO₂Cl: 423.1965; found: 424.2042 (M+H).

The enantiomer eluting later was collected as Example 92. HRMS calculated for C₂₆H₃₀NO₂Cl: 423.1965; found: 424.2039 (M+H).

The fractions containing the partially saturated compound from prep RP-HPLC were further purified by chiral chromatography, column: AD, 50×500 mm, 20 μm, eluents: EtOH/heptane+0.05% HCOOH to obtain Example 93. HRMS calculated for C₂₆H₂₈NO₂Cl: 421.1808; found: 422.1875 (M+H).

### Example 94 (1s,4s)-4-(3-bromoanilino)-6′-methoxy-3′H-dispiro[cyclohexane-1,1′-indene-2′,1″-cyclopentane]-4-carboxylic acid

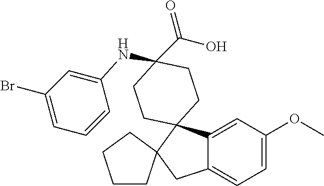 [SMILES: COc1ccc2c(c1)[C@]1(CC[C@](Nc3cccc(Br)c3)(C(=O)O)CC1)C1(CCCC1)C2]

and

### Example 95 (1r,4r)-4-(3-bromoanilino)-6′-methoxy-3′H-dispiro[cyclohexane-1,1′-indene-2′,1″-cyclopentane]-4-carboxylic acid

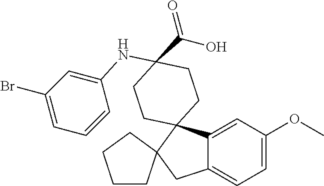 [SMILES: COc1ccc2c(c1)[C@]1(CC[C@@](Nc3cccc(Br)c3)(C(=O)O)CC1)C1(CCCC1)C2]

A solution of Example 89D (864 mg, 3.04 mmol, 1 eq) in THE (50 mL) was treated as described in General procedure 10 using 3-bromoaniline (364 μL, 3.34 mmol, 1.1 eq) and the mixture stirred at rt for 18 h. A mixture of diastereoisomers was obtained. The mixture was partitioned between DCM and water, the pH adjusted to 4 with 1 M aq. HCl solution and extracted with DCM. The organic phase was washed with brine, dried (MgSO₄) and concentrated in vacuo. Purification by automated flash column chromatography (CombiFlash Rf, 40 g RediSep™ silica cartridge) eluting with a gradient of 0-40% EtOAc in heptane was followed by preparative HPLC at pH 9. The diastereoisomer eluting earlier was collected as Example 94 as a white solid (2.8 mg, 0.01 mmol). LRMS calculated for C₂₆H30BrNO₃: 483; found: 484 (M+H). ¹H NMR (400 MHz, DMSO-d₆) δ ppm: 7.08 (d, J=8.1 Hz, 1H), 6.99 (t, J=8.1 Hz, 1H), 6.92 (d, J=2.3 Hz, 1H), 6.81 (t, J=2.0 Hz, 1H), 6.72-6.59 (m, 3H), 3.72 (s, 3H), 2.61 (s, 2H), 2.38-2.28 (m, 2H), 1.86-1.69 (m, 4H), 1.69-1.45 (m, 8H), 1.32-1.22 (m, 2H).

The diastereoisomer eluting later was collected as Example 95 as a white solid (3.7 mg, 0.01 mmol). LRMS calculated for C₂₆H30BrNO₃: 483; found: 484 (M+H). ¹H NMR (400 MHz, DMSO-d₆) δ ppm: 7.08 (d, J=8.2 Hz, 1H), 7.02-6.96 (m, 2H), 6.74 (t, J=2.0 Hz, 1H), 6.69 (dd, J=8.1, 2.2 Hz, 1H), 6.65 (dd, J=7.9, 1.4 Hz, 1H), 6.53 (dd, J=8.3, 2.2 Hz, 1H), 6.20 (br s, 1H), 3.73 (s, 3H), 2.60 (s, 2H), 2.14-1.98 (m, 4H), 1.79 (dt, J=13.7, 7.0 Hz, 2H), 1.69-1.40 (m, 8H), 1.28-1.18 (m, 2H).

### Example 96 (1r,4r)-4-(3-chloroanilino)-2′-cyclobutylspiro[cyclohexane-1,1′-indene]-4-carboxylic acid

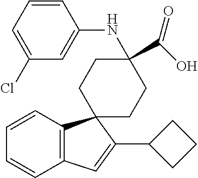 [SMILES: O=C(O)[C@]1(Nc2cccc(Cl)c2)CC[C@]2(CC1)C(C1CCC1)=Cc1ccccc12]

To an oven-dried microwave vial was added Preparation 6a (260 mg, 0.6 mmol, 1 eq) and AtaPhos (9 mg, 0.01 mmol, 0.02 eq) in THE (2 mL), followed by 1-methylimidazole (96 μL, 1.2 mmol, 2 eq) and cyclobutylzinc bromide (2.86 mL, 0.5 M in THF, 1.2 mmol, 2 eq). The mixture was sparged with N₂ (10 min) and heated at 120° C. for 1 h under microwave irradiation. The reaction was partitioned between DCM and water, and the organic phase was washed with 1 M aq. HCl solution, brine, dried (PTFE phase separator) and concentrated in vacuo. Purification by reverse phase automated flash chromatography (CombiFlash Rf, C18 13 g RediSep column) eluting with a gradient of 35-100% MeCN in water afforded Example 96 as a white glass (83 mg, 0.2 mmol, 34%). LRMS calculated for C₂₅H₂₆ClNO₂: 407; found: 408 (M+H). ¹H NMR (400 MHz, DMSO-d₆) δ ppm: 12.77 (br s, 1H), 7.64 (d, J=7.5 Hz, 1H), 7.30 (dd, J=7.4, 1.2 Hz, 1H), 7.23 (td, J=7.4, 0.9 Hz, 1H), 7.15-7.07 (m, 2H), 6.65 (t, J=2.1 Hz, 1H), 6.61-6.55 (m, 3H), 6.35 (br s, 1H), 3.27-3.16 (m, 1H), 2.43-2.30 (m, 2H), 2.29-1.92 (m, 9H), 1.85-1.76 (m, 1H), 0.93-0.80 (m, 2H).

### Example 97 (1r,4r)-2′-[3-(benzyloxy)cyclobutyl]-4-(3-chloroanilino)spiro[cyclohexane-1,1′-indene]-4-carboxylic acid

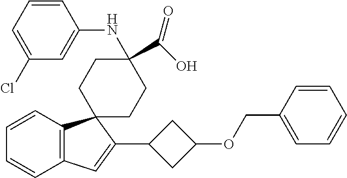 [SMILES: O=C(O)[C@]1(Nc2cccc(Cl)c2)CC[C@]2(CC1)C(C1CC(OCc3ccccc3)C1)=Cc1ccccc12]

To an oven-dried Schlenk flask was added zinc (1.02 g, 15.6 mmol, 1.5 eq) and LiCl (659 mg, 15.6 mmol, 1.5 eq) and the vessel was heated at 160° C. for 20 min under vacuum then allowed to cool to rt and placed under a N₂ atmosphere. 1,2-dibromoethane (36 μL, 0.41 mmol, 0.04 eq) was added and the mixture was heated at 60° C. for 15 min. The flask was removed from the heat and TMS-Cl (11 μL, 0.08 mmol, 0.01 eq) was added, followed by 0.5 M I₂ in THF (0.08 mL). The mixture was heated at 60° C. for 10 min then allowed to cool to rt before the addition of ({[(1r,3r)-3-bromocyclobutyl]oxy}methyl)benzene (2.5 g, 10.4 mmol, 1 eq) in THE (4 mL). The mixture was heated at 50° C. for 18 h. The resultant mixture was allowed to cool to rt and cannulation through a filter (cotton-wool/celite/cotton-wool) into a dry Schlenk tube afforded [(1r,3r)-3-(benzyloxy)cyclobutyl](bromido)zinc as a 0.2 M solution (as determined by titration with a 0.5 M solution of iodine). Then to an oven-dried microwave vial was added Preparation 6a (100 mg, 0.23 mmol, 1 eq) and AtaPhos (8 mg, 11.6 μmol, 0.05 eq) in THE (1 mL), followed by 1-methylimidazole (37 μL, 0.46 mmol, 2 eq) and the previously obtained [(1r,3r)-3-(benzyloxy)cyclobutyl](bromido)zinc solution (2.31 mL, 0.2 M, 0.46 mmol, 2 eq). The mixture was sparged with N₂ (10 min) and heated at 130° C. for 2 h under microwave irradiation. The reaction was partitioned between DCM and water, and the organic phase was washed with 1 M aq. HCl solution, brine, dried (PTFE phase separator) and concentrated in vacuo. Purification by reverse phase automated flash chromatography (CombiFlash Rf, C18 4.3 g RediSep column) eluting with a gradient of 40-100% MeCN in water afforded a 6:1 mixture of trans/cis isomers Example 97 as a white powder (10 mg, 0.02 mmol, 8%). LRMS calculated for C₃₂H₃₂ClNO₃: 513; found: 514 (M+H). ¹H NMR (400 MHz, DMSO-d₆) δ ppm: 12.86 (br s, 1H), 7.65 (d, J=7.5 Hz, 1H), 7.40-7.26 (m, 6H), 7.25-7.19 (m, 1H), 7.15-7.05 (m, 2H), 6.68-6.54 (m, 4H), 6.34 (br s, 1H), 4.42 (s, 2H), 4.33-4.25 (m, 1H), 2.44-2.31 (m, 4H), 2.29-2.02 (m, 6H), 0.92-0.83 (m, 2H).

### Example 98

### Example 98A [1-(tert-butoxycarbonyl)azetidin-3-yl](iodido)zinc

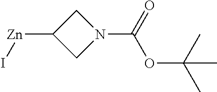

To an oven-dried Schlenk flask was added zinc (1.4 g, 21.4 mmol, 1.25 eq) and the vessel was then heated under vacuum with a heat gun for 5 min, allowed to cool to rt and purged with N₂. DMA (10 mL) was added and the suspension gently stirred, followed by the addition of a 7:5 (v/v) mixture of TMS-Cl and dibromoethane (0.43 mL), which produced an exotherm gas evolution. The mixture was stirred at rt for 10 min and then a solution of tert-butyl 3-iodoazetidine-1-carboxylate (4.85 g, 17.1 mmol, 1 eq) in DMA (7 mL) was added dropwise, maintaining the internal temperature below 45° C. The mixture was stirred at rt for 45 min and then allowed to stand for 18 h. The resultant mixture was transferred via cannulation through a filter (cotton-wool/celite/cotton-wool) into a dry Schlenk tube to afford Example 98A as a 0.34 M solution (as determined by titration with a 0.5 M solution of iodine) that was used without further characterisation.

### Example 98B tert-butyl 3-[(1r,4r)-4-(3-chloroanilino)-4-(methoxycarbonyl)spiro[cyclohexane-1,1′-inden]-2′-yl]azetidine-1-carboxylate

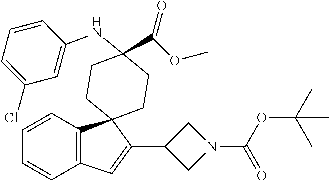 [SMILES: COC(=O)[C@]1(Nc2cccc(Cl)c2)CC[C@]2(CC1)C(C1CN(C(=O)OC(C)(C)C)C1)=Cc1ccccc12]

To an oven-dried sealed tube was added Preparation 6c (500 mg, 1.12 mmol, 1 eq) and AtaPhos (16 mg, 0.02 mmol, 0.02 eq) in THE (5 mL), followed by 1-methylimidazole (178 μL, 2.24 mmol, 2 eq) and Example 98A (6.58 mL, 0.34 M, 2.24 mmol, 2 eq). The mixture was sparged with N₂ (10 min) and heated at 75° C. for 18 h. The reaction was partitioned between EtOAc and water, and the organic phase was washed with 1 M aq. HCl solution, brine, dried (MgSO₄) and concentrated in vacuo. Purification by automated flash chromatography (CombiFlash Rf, 12 g RediSep™ silica cartridge) eluting with a gradient of 0-30% EtOAc in heptane afforded Example 98B as a colourless glass (300 mg, 0.57 mmol, 51%). LRMS calculated for C₃₀H₃₅ClN₂O₄: 522; found: 523 (M+H). ¹H NMR (400 MHz, DMSO-d₆) δ ppm: 7.66 (d, J=7.5 Hz, 1H), 7.36 (dd, J=7.5, 1.2 Hz, 1H), 7.27 (td, J=7.5, 0.9 Hz, 1H), 7.16 (td, J=7.5, 1.2 Hz, 1H), 7.11 (t, J=8.1 Hz, 1H), 6.80 (s, 1H), 6.67 (t, J=2.1 Hz, 1H), 6.61 (dd, J=7.9, 2.0 Hz, 1H), 6.49 (dd, J=8.3, 2.3 Hz, 1H), 6.39 (s, 1H), 4.24-4.08 (m, 2H), 3.82 (t, J=7.4 Hz, 2H), 3.71 (s, 3H), 3.58-3.48 (m, 1H), 2.38 (td, J=13.9, 3.8 Hz, 2H), 2.23-2.14 (m, 2H), 2.05-1.91 (m, 2H), 1.40 (s, 9H), 0.97-0.88 (m, 2H).

### Example 98C methyl (1r,4r)-2′-(azetidin-3-yl)-4-(3-chloroanilino)spiro[cyclohexane-1,1′-indene]-4-carboxylate

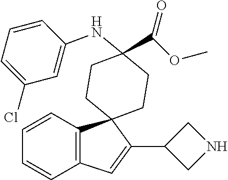 [SMILES: COC(=O)[C@]1(Nc2cccc(Cl)c2)CC[C@]2(CC1)C(C1CNC1)=Cc1ccccc12]

To a solution of Example 98B (300 mg, 0.57 mmol, 1 eq) in DCM (7 mL) was added TFA (1 mL) and the mixture was stirred at rt for 5 h. The reaction was partitioned between DCM and sat. aq. NaHCO₃ solution, and the organic phase was washed with brine, dried (MgSO₄) and concentrated in vacuo to afford Example 98C as a yellow glass (209 mg, 0.49 mmol, 86%) that was used directly in the subsequent step without further purification. LRMS calculated for C₂₅H₂₇ClN₂O₂: 422; found: 423 (M+H).

### Example 98D methyl (1r,4r)-4-(3-chloroanilino)-2′-{1-[(pyridin-4-yl)methyl]azetidin-3-yl}spiro[cyclohexane-1,1′-indene]-4-carboxylate

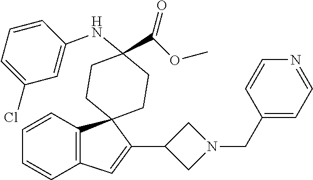 [SMILES: COC(=O)[C@]1(Nc2cccc(Cl)c2)CC[C@]2(CC1)C(C1CN(Cc3ccncc3)C1)=Cc1ccccc12]

To a solution of Example 98C (70 mg, 0.17 mmol, 1 eq) in THE (2 mL), with 4\acute{Å} molecular sieves, was added 4-formylpyridine (0.02 mL, 0.2 mmol, 1.2 eq) followed by NaBH(OAc)₃ (70 mg, 0.33 mmol, 2 eq) and the mixture was stirred at rt for 5 h. The mixture was partitioned between DCM and sat. aq. NaHCO₃ solution, the organic phase was washed with brine, dried (PTFE phase separator) and concentrated in vacuo. Purification by automated flash chromatography (CombiFlash Rf, 4 g RediSep™ silica cartridge) eluting with a gradient of 0-10% MeOH in DCM afforded Example 98D as a colourless glass (42 mg, 0.08 mmol, 49%). LRMS calculated for C₃₁H₃₂ClN₃O₂: 513; found: 514 (M+H). ¹H NMR (400 MHz, DMSO-d₆) δ ppm: 8.53-8.48 (m, 2H), 7.64 (d, J=7.6 Hz, 1H), 7.36-7.30 (m, 3H), 7.28-7.23 (m, 1H), 7.17-7.08 (m, 2H), 6.67 (s, 1H), 6.65 (t, J=2.1 Hz, 1H), 6.60 (dd, J=7.7, 1.9 Hz, 1H), 6.48 (dd, J=8.2, 2.2 Hz, 1H), 6.40 (s, 1H), 3.70 (s, 3H), 3.69-3.62 (m, 4H), 3.44-3.34 (m, 1H), 3.05 (t, J=7.3 Hz, 2H), 2.42-2.32 (m, 2H), 2.22-2.13 (m, 2H), 2.09-1.97 (m, 2H), 0.93-0.84 (m, 2H).

### Example 98 (1r,4r)-4-(3-chloroanilino)-2′-{1-[(pyridin-4-yl)methyl]azetidin-3-yl}spiro[cyclohexane-1,1′-indene]-4-carboxylic acid

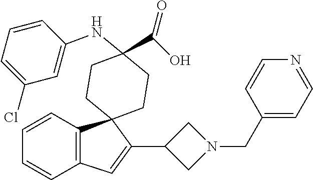 [SMILES: O=C(O)[C@]1(Nc2cccc(Cl)c2)CC[C@]2(CC1)C(C1CN(Cc3ccncc3)C1)=Cc1ccccc12]

To a solution of Example 98D (42 mg, 0.08 mmol, 1 eq) in MeOH (2 mL) was added LiOH×H₂O (24 mg, 0.57 mmol, 7 eq) and the mixture was heated at 120° C. for 40 min under microwave irradiation. Purification by reverse phase automated flash chromatography (CombiFlash Rf, C18 4.3 g RediSep column) eluting with a gradient of 10-70% MeCN in water afforded Example 98 as a white powder (22.2 mg, 0.04 mmol, 54%). LRMS calculated for C₃₀H₃₀ClN₃O₂: 499; found: 500 (M+H). ¹H NMR (400 MHz, DMSO-d₆) δ ppm: 8.52-8.47 (m, 2H), 7.77 (d, J=7.6 Hz, 1H), 7.35-7.27 (m, 3H), 7.21 (td, J=7.4, 0.9 Hz, 1H), 7.07 (td, J=7.5, 1.3 Hz, 1H), 6.94 (t, J=8.0 Hz, 1H), 6.66 (t, J=2.0 Hz, 1H), 6.64-6.59 (m, 2H), 6.40-6.35 (m, 1H), 5.86 (s, 1H), 3.70-3.64 (m, 2H), 3.63 (s, 2H), 3.47-3.37 (m, 1H), 3.07-3.00 (m, 2H), 2.42-2.29 (m, 2H), 2.06-1.89 (m, 4H), 0.81-0.72 (m, 2H).

### Example 99

### Example 99A methyl (1r,4r)-4-(3-chloroanilino)-2′-{1-[(2,3,6-trifluorophenyl)methyl]azetidin-3-yl}spiro[cyclohexane-1,1′-indene]-4-carboxylate

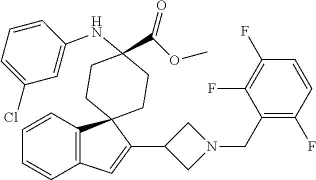 [SMILES: COC(=O)[C@]1(Nc2cccc(Cl)c2)CC[C@]2(CC1)C(C1CN(Cc3c(F)ccc(F)c3F)C1)=Cc1ccccc12]

To a solution of Example 98C (70 mg, 0.17 mmol, 1 eq) in THF (2 mL), with 4× molecular sieves, was added 2,3,6-trifluorobenzaldehyde (0.02 mL, 0.2 mmol, 1.2 eq) followed by NaBH(OAc)₃ (70 mg, 0.33 mmol, 2 eq) and the mixture was stirred at rt for 5 h. The mixture was partitioned between DCM and sat. aq. NaHCO₃ solution, the organic phase was washed with brine, dried (PTFE phase separator) and concentrated in vacuo. Purification by automated flash chromatography (CombiFlash Rf, 4 g RediSep™ silica cartridge) eluting with a gradient of 0-7% MeOH in DCM afforded Example 99A as a colourless glass (49 mg, 0.09 mmol, 52%). LRMS calculated for C₃₂H₃₀ClF₃N₂O₂: 566; found: 567 (M+H). ¹H NMR (400 MHz, DMSO-d₆) δ ppm: 7.63 (d, J=7.5 Hz, 1H), 7.52-7.42 (m, 1H), 7.34 (dd, J=7.5, 1.2 Hz, 1H), 7.25 (td, J=7.5, 0.9 Hz, 1H), 7.20-7.06 (m, 3H), 6.64-6.57 (m, 3H), 6.49-6.44 (m, 1H), 6.37 (s, 1H), 3.70 (s, 3H), 3.66 (s, 2H), 3.64-3.58 (m, 2H), 3.33-3.25 (m, 1H), 3.12-3.05 (m, 2H), 2.42-2.29 (m, 2H), 2.22-2.12 (m, 2H), 2.06-1.95 (m, 2H), 0.92-0.83 (m, 2H).

### Example 99 (1r,4r)-4-(3-chloroanilino)-2′-{1-[(2,3,6-trifluorophenyl)methyl]azetidin-3-yl}spiro[cyclohexane-1,1′-indene]-4-carboxylic acid

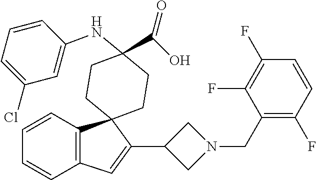 [SMILES: O=C(O)[C@]1(Nc2cccc(Cl)c2)CC[C@]2(CC1)C(C1CN(Cc3c(F)ccc(F)c3F)C1)=Cc1ccccc12]

To a solution of Example 99A (49 mg, 0.09 mmol, 1 eq) in MeOH (2 mL) was added LiOH×H₂O (25.4 mg, 0.6 mmol, 7 eq) and the mixture was heated at 120° C. for 40 min under microwave irradiation. Purification by reverse phase automated flash chromatography (CombiFlash Rf, C18 4.3 g RediSep column) eluting with a gradient of 10-70% MeCN in water afforded Example 99 as a white powder (21.7 mg, 0.04 mmol, 45%). LRMS calculated for C₃₁H₂₈ClF₃N₂O₂: 552; found: 553 (M+H). ¹H NMR (400 MHz, DMSO-d₆) δ ppm: 7.75 (d, J=7.5 Hz, 1H), 7.52-7.41 (m, 1H), 7.30 (dd, J=7.4, 1.2 Hz, 1H), 7.21 (td, J=7.5, 0.8 Hz, 1H), 7.19-7.12 (m, 1H), 7.08 (td, J=7.5, 1.2 Hz, 1H), 6.94 (t, J=8.0 Hz, 1H), 6.63 (t, J=2.1 Hz, 1H), 6.61-6.57 (m, 1H), 6.54 (s, 1H), 6.38 (dd, J=7.8, 1.9 Hz, 1H), 5.84 (br s, 1H), 3.66 (s, 2H), 3.64-3.58 (m, 2H), 3.10-3.03 (m, 2H), 2.40-2.28 (m, 2H), 2.05-1.86 (m, 4H), 0.80-0.70 (m, 2H).

### Example 100 (1r,2′S,4S)-4-(3-chloroanilino)-2′-cyclobutyl-2′,3′-dihydrospiro[cyclohexane-1,1′-indene]-4-carboxylic acid

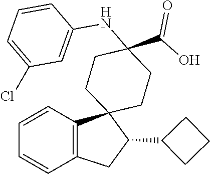 [SMILES: O=C(O)[C@]1(Nc2cccc(Cl)c2)CC[C@@]2(CC1)c1ccccc1C[C@H]2C1CCC1]

and

### Example 101 (1r,2′R,4R)-4-(3-chloroanilino)-2′-cyclobutyl-2′,3′-dihydrospiro[cyclohexane-1,1′-indene]-4-carboxylic acid

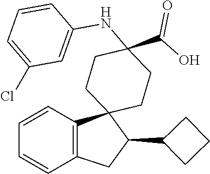 [SMILES: O=C(O)[C@]1(Nc2cccc(Cl)c2)CC[C@@]2(CC1)c1ccccc1C[C@@H]2C1CCC1]

To a solution of Example 96 (75 mg, 0.18 mmol, 1 eq) in EtOH (3 mL) and THE (3 mL) was added catalytic Pt/C (35 mg, 0.01 mmol, 0.05 eq) under a N₂ atmosphere. The mixture was evacuated and backfilled with N₂, then evacuated and backfilled with H₂ and shaken for 18 h at rt under an atmosphere of H₂. The reaction was filtered through celite, eluted with EtOH and evaporated under reduced pressure. Purification by reverse phase automated flash chromatography (CombiFlash Rf, C18 4.3 g RediSep column) eluting with a gradient of 35-100% MeCN in water afforded a racemate. The enantiomers were separated by chiral chromatography. Column: AD, 50×500 mm, 20 μm. Eluents: 15:85 EtOH/Heptane+0.05% HCOOH. The enantiomer eluting earlier was collected as Example 100. ¹H NMR (500 MHz, DMSO-d₆) δ ppm: 12.63 (br s, 1H), 7.35 (dm, 1H), 7.21-7.08 (m, 3H), 7.05 (t, 1H), 6.59 (t, 1H), 6.53 (dm, 1H), 6.53 (dm, 1H), 6.2 (br s, 1H), 2.85/2.49 (dd+dd, 2H), 2.39-1.34 (m, 14H), 2.36 (m, 1H), 2.02 (m, 1H). HRMS calculated for C₂₅H₂₈ClNO₂: 409.1808; found: 410.1879 (M+H).

The enantiomer eluting later was collected as Example 101. HRMS calculated for C₂₅H₂₈ClNO₂: 409.1808; found: 410.1876 (M+H).

### Example 102

### Example 102A methyl (1r,4r)-4-(3-chloroanilino)-2′-[1-(2,2,2-trifluoroethyl)azetidin-3-yl]spiro[cyclohexane-1,1′-indene]-4-carboxylate

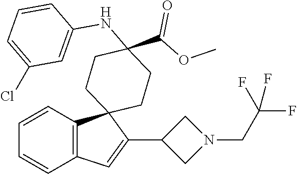 [SMILES: COC(=O)[C@]1(Nc2cccc(Cl)c2)CC[C@]2(CC1)C(C1CN(CC(F)(F)F)C1)=Cc1ccccc12]

To a solution of Example 98C (75 mg, 0.18 mmol, 1 eq) in DCM (3 mL) was added TEA (74 μL, 0.53 mmol, 3 eq) followed by 2,2,2-trifluoroethyl trifluoromethanesulphonate (36 μL, 0.27 mmol, 1.5 eq). The mixture was stirred at rt for 18 h. The reaction was partitioned between DCM and sat. aq. NaHCO₃ solution, and the organic phase was washed with brine, dried (PTFE phase separator) and concentrated in vacuo. Purification by automated flash chromatography (CombiFlash Rf, 4 g RediSep™ silica cartridge) eluting with a gradient of 0-80% EtOAc in heptane afforded Example 102A as a colourless glass (36 mg, 0.07 mmol, 40%). LRMS calculated for C₂₇H₂₈ClF₃N₂O₂: 504; found: 505 (M+H). ¹H NMR (400 MHz, DMSO-d₆) δ ppm: 7.65 (d, J=7.5 Hz, 1H), 7.36-7.32 (m, 1H), 7.25 (td, J=7.5, 0.9 Hz, 1H), 7.15 (td, J=7.5, 1.2 Hz, 1H), 7.11 (t, J=8.1 Hz, 1H), 6.68 (s, 1H), 6.66 (t, J=2.1 Hz, 1H), 6.63-6.59 (m, 1H), 6.52-6.47 (m, 1H), 6.42 (s, 1H), 3.79-3.73 (m, 2H), 3.71 (s, 3H), 3.48-3.37 (m, 1H), 3.28-3.18 (m, 4H), 2.43-2.32 (m, 2H), 2.24-2.14 (m, 2H), 2.08-1.96 (m, 2H), 0.93-0.85 (m, 2H).

### Example 102 (1r,4r)-4-(3-chloroanilino)-2′-[1-(2,2,2-trifluoroethyl)azetidin-3-yl]spiro[cyclohexane-1,1′-indene]-4-carboxylic acid

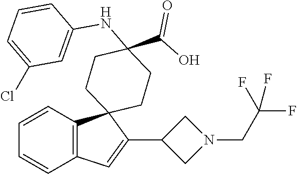 [SMILES: O=C(O)[C@]1(Nc2cccc(Cl)c2)CC[C@]2(CC1)C(C1CN(CC(F)(F)F)C1)=Cc1ccccc12]

To a solution of Example 102A (36 mg, 0.07 mmol, 1 eq) in MeOH (2 mL) was added LiOH×H₂O (18 mg, 0.43 mmol, 6 eq) and the mixture was heated at 110° C. for 30 min under microwave irradiation. The reaction was allowed to cool to rt and partitioned between DCM and water, and the organic phase was washed with 1 M aq. HCl solution, brine, dried (PTFE phase separator) and concentrated in vacuo. Purification by reverse phase automated flash chromatography (CombiFlash Rf, C18 4.3 g RediSep column) eluting with a gradient of 25-100% MeCN in water afforded Example 102 as a white powder (14.6 mg, 0.03 mmol, 42%). LRMS calculated for C₂₆H₂₆ClF₃N₂O₂: 490; found: 491 (M+H). ¹H NMR (400 MHz, DMSO-d₆) δ ppm: 12.87 (br s, 1H), 7.65 (d, J=7.5 Hz, 1H), 7.33 (dd, J=7.5, 1.2 Hz, 1H), 7.25 (td, J==7.5, 0.9 Hz, 1H), 7.14 (td, J=7.5, 1.2 Hz, 1H), 7.10 (t, J=8.1 Hz, 1H), 6.69-6.65 (m, 2H), 6.62-6.55 (m, 2H), 6.32 (br s, 1H), 3.76 (t, J=6.8 Hz, 2H), 3.49-3.38 (m, 1H), 3.28-3.18 (m, 4H), 2.42-2.30 (m, 2H), 2.21-2.12 (m, 2H), 2.07-1.94 (m, 2H), 0.92-0.82 (m, 2H).

### Example 103 and Example 104 and Example 105 and Example 106

### Example 103A tert-butyl(dimethyl){[rac-(1R,2R)-2-(4,4,5,5-tetramethyl-1,3,2-dioxaborolan-2-yl)cyclopropyl]methoxy}silane

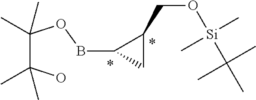

1 M 3-azabicyclo[2.2.2]oct-5-en-2-one solution in heptane (6.7 mL, 6.7 mmol) was dissolved in dry DCM (6.7 mL) and cooled to 0° C. TFA (513 μL, 6.7 mmol) in DCM (3.35 mL) was added to the mixture and stirred at 0° C. for 30 min. CH₂I₂ (1.8 g, 6.7 mmol) in DCM (3.35 mL) was added dropwise and stirred at 0° C. for 30 min. Tert-butyl-dimethyl-[(E)-3-(4,4,5,5-tetramethyl-1,3,2-dioxaborolan-2-yl)allyloxy]silane (1.0 g, 3.35 mmol) in DCM (6.7 mL) was added to the mixture and stirred at rt for 2 h. The reaction was quenched with water and extracted with DCM. The combined organic layers were washed with brine, dried over Na₂SO₄, filtered and the filtrate was concentrated under reduced pressure to give Example 103A as a racemic single diastereoisomer. ¹H NMR (400 MHz, CDCl₃) δ ppm: 3.61 (dd, 1H), 3.47 (dd, 1H), 1.30-1.24 (m, 2H), 1.23 (s, 12H), 0.90 (s, 9H), 0.73-0.68 (m, 1H), 0.59-0.54 (m, 1H), 0.06 (s, 6H).

### Example 103B rac-methyl (1r,4r)-2′-[(1R*,2R*)-2-({[tert-butyl(dimethyl)silyl]oxy}methyl)cyclopropyl]-4-(3-chloroanilino)spiro[cyclohexane-1,1′-indene]-4-carboxylate

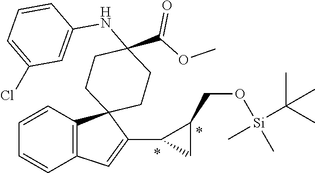 [SMILES: COC(=O)[C@]1(Nc2cccc(Cl)c2)CC[C@]2(CC1)C([C@H]1C[C@@H]1CO[Si](C)(C)C(C)(C)C)=Cc1ccccc12]

Preparation 6c (5.0 g, 11.2 mmol) was dissolved in THE (80 mL) and water (20 mL), then Example 103A (7.0 g, 22.4 mmol), Pd(PPh₃)₄ (646 mg, 0.56 mmol) and Na₂CO₃ (3.56 g, 33.6 mmol) were added and the mixture was stirred under N₂ atmosphere at 100° C. overnight. Then it was concentrated under reduced pressure and purified via flash chromatography using heptane and EtOAc as eluents to obtain Example 103B as a racemic single diastereoisomer. LRMS calculated for C₃₂H₄₂ClNO₃Si: 551.26; found 552.3 (M+H).

### Example 103C rac-methyl (1r,4r)-4-(3-chloroanilino)-2′-[(1R*,2S*)-2-(hydroxymethyl)cyclopropyl]-2′,3′-dihydrospiro[cyclohexane-1,1′-indene]-4-carboxylate, diastereoisomer 1

and

### Example 103D rac-methyl (1r,4r)-4-(3-chloroanilino)-2′-[(1R*,2S*)-2-(hydroxymethyl)cyclopropyl]-2′,3′-dihydrospiro[cyclohexane-1,1′-indene]-4-carboxylate, diastereoisomer 2

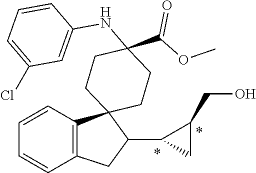

Example 103B (6.04 g, 11.0 mmol) was dissolved in EtOH (100 mL) and AcOH (10 mL). 10% Pt/C (200 mg, 0.03 g/g indene) was added to the mixture. The flask was evacuated and backfilled with N₂ (×3), then evacuated and filled with H₂ (1 bar) and the mixture was stirred at rt overnight. Then it was filtered, washed with EtOH, and the filtrate was concentrated under reduced pressure. The crude product was purified via flash chromatography using heptane and EtOAc as eluents. The racemic diastereoisomer eluting earlier was collected as Example 103C. LRMS calculated for C₂₆H₃₀ClNO₃: 439.19; found 440.2 (M+H).

The racemic diastereoisomer eluting later was collected as Example 103D. LRMS calculated for C₂₆H₃₀ClNO₃: 439.19; found 440.3 (M+H).

### Example 103 (1r,4r)-4-(3-chloroanilino)-2′-[(1R*,2S*)-2-{[(thieno[3,2-b]pyridin-7-yl)oxy]methyl}cyclopropyl]-2′,3′-dihydrospiro[cyclohexane-1,1′-indene]-4-carboxylic acid, diastereoisomer 1 enantiomer 1

### Example 104 (1r,4r)-4-(3-chloroanilino)-2′-[(1R*,2S*)-2-{[(thieno[3,2-b]pyridin-7-yl)oxy]methyl}cyclopropyl]-2′,3′-dihydrospiro[cyclohexane-1,1′-indene]-4-carboxylic acid, diastereoisomer 2 enantiomer 1

and

### Example 105 (1r,4r)-4-(3-chloroanilino)-2′-[(1R*,2S*)-2-{[(thieno[3,2-b]pyridin-7-yl)oxy]methyl}cyclopropyl]-2′,3′-dihydrospiro[cyclohexane-1,1′-indene]-4-carboxylic acid, diastereoisomer 1 enantiomer 2

and

### Example 106 (1r,4r)-4-(3-chloroanilino)-2′-[(1R*,2S*)-2-{[(thieno[3,2-b]pyridin-7-yl)oxy]methyl}cyclopropyl]-2′,3′-dihydrospiro[cyclohexane-1,1′-indene]-4-carboxylic acid, diastereoisomer 2 enantiomer 2

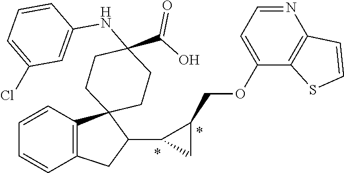

Using General procedure 32 and Example 103C as the appropriate indane and thieno[3,2-b]pyridin-7-ol as the appropriate alcohol, a racemic distereoisomer was obtained. The enantiomers were separated by chiral chromatography. Column: AD, 50×500 mm, 20 μm, eluents: EtOH+0.05% HCOOH. The enantiomer eluting earlier was collected and purified via prep RP-HPLC using 25 mM aq. NH₄HCO₃ solution and MeCN as eluents resulting Example 103. ¹H NMR (500 MHz, DMSO-d₆) δ ppm: 11.93 (br s, 1H), 8.46 (d, 1H), 8.04 (d, 1H), 7.50 (d, 1H), 7.31 (d, 1H), 7.22 (d, 1H), 7.16 (m, 1H), 7.13 (m, 1H), 7.01 (t, 1H), 6.87 (d, 1H), 6.66 (t, 1H), 6.55 (dd, 1H), 6.51 (dd, 1H), 6.26 (br s, 1H), 4.29/4.03 (m+m, 2H), 3.07/2.69 (dd+dd, 2H), 2.65-1.35 (m, 8H), 1.62 (m, 1H), 1.43 (m, 1H), 0.74 (m, 1H), 0.54/0.49 (m+m, 2H). HRMS calculated for C₃₂H₃₁N₂O₃SCl: 558.1744; found: 559.1813 (M+H).

The enantiomer eluting later was collected and further purified via prep RP-HPLC using 25 mM aq. NH₄HCO₃ solution and MeCN as eluents resulting Example 105. HRMS calculated for C₃₂H₃₁N₂O₃SCl: 558.1744; found: 559.1817 (M+H).

Using General procedure 32 and Example 103D as the appropriate indane and thieno[3,2-b]pyridin-7-ol as the appropriate alcohol, a racemic distereoisomer was obtained. The enantiomers were separated by chiral chromatography. Column: AD, 50×500 mm, 20 μm, eluents: EtOH+0.05% HCOOH. The enantiomer eluting earlier was collected and purified via prep RP-HPLC using 25 mM aq. NH₄HCO₃ solution and MeCN as eluents resulting Example 104. ¹H NMR (500 MHz, DMSO-d₆) δ ppm: 8.47 (d, 1H), 8.07 (d, 1H), 7.51 (d, 1H), 7.26 (d, 1H), 7.16 (d, 1H), 7.13 (t, 1H), 7.10 (t, 1H), 7.04 (t, 1H), 6.88 (d, 1H), 6.64 (t, 1H), 6.55 (dm, 1H), 6.53 (dm, 1H), 4.39/3.82 (dd+dd, 2H), 3.06/2.75 (dd+dd, 2H), 2.36-1.39 (m, 8H), 1.68 (m, 1H), 1.12 (m, 1H), 0.78/0.72 (m+m, 2H), 0.72 (m, 1H). HRMS calculated for C₃₂H₃₁N₂O₃SCl: 558.1744; found: 559.1812 (M+H).

The enantiomer eluting later was collected and purified via prep RP-HPLC using 25 mM aq. NH₄HCO₃ solution and MeCN as eluents resulting Example 106. HRMS calculated for C₃₂H₃₁N₂O₃SCl: 558.1744; found: 559.1816 (M+H).

### Example 107 and Example 108

### Example 107A dispiro[[1,3]dioxolane-2,1′-cyclohexane-4′,1″-indene]

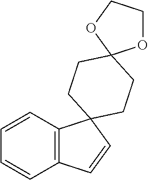 [SMILES: C1=CC2(CCC3(CC2)OCCO3)c2ccccc21]

Using General procedure 8a and 1H-indene as the appropriate indene Example 107A was obtained. MS (EI, 70 eV) m/z (% relative intensity, [ion]): 86 (35), 99 (38), 128 (100), 141 (44), 180 (46), 242 (15, [M+]).

### Example 107B rac-(1aR,6aS)-1a,6a-dihydro-1H-dispiro[cyclopropa[a]indene-6,1′-cyclohexane-4′,2″-[1,3]dioxolane]

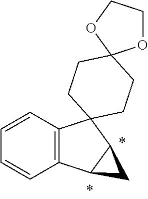 [SMILES: c1ccc2c(c1)[C@@H]1C[C@@H]1C21CCC2(CC1)OCCO2]

A dried flask was charged with DCM (8 mL) and Et₂Zn (1 M in heptane, 8.26 mL, 8.26 mmol) under N₂ atmosphere and cooled to 0° C. DCM (3 mL) solution of TFA (0.63 mL, 8.26 mmol) was added dropwise. After 20 min stirring DCM (2 mL) solution of CH₂I₂ (0.67 mL, 8.26 mmol) was added. After 20 min stirring at 0° C. Example 107A (1 g, 4.13 mmol) was added and the mixture was allowed to warm up to rt. After overnight stirring almost 60% conversion was observed. The reaction was quenched with sat. aq. NaHCO₃ and water. The precipitation was filtered off, and the filtrate was extracted with DCM. The combined organic layers were dried over Na₂SO₄, filtered and the filtrate was concentrated under reduced pressure to yield Example 107B, which was used in the next step without purification. MS (EI, 70 eV) m/z (% relative intensity, [ion]): 99 (81), 115 (34), 128 (30), 141 (100), 155 (29), 179 (22), 194 (25), 228 (23), 256 (19, [M+]).

### Example 107C rac-(1′aR,6′aS)-1′a,6′a-dihydro-1′H-spiro[cyclohexane-1,6′-cyclopropa[a]inden]-4-one

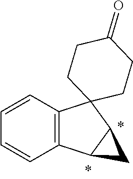 [SMILES: O=C1CCC2(CC1)c1ccccc1[C@@H]1C[C@@H]12]

Using General procedure 9 and Example 107B as the appropriate ketal, then purifying the crude product via prep RP-HPLC using 25 mM aq. NH₄HCO₃ solution and MeCN as eluents, Example 107C was obtained. ¹H NMR (500 MHz, DMSO-d₆) δ ppm: 7.28 (m, 1H), 7.16 (m, 1H), 7.10 (m, 1H), 7.07 (m, 1H), 2.88 (td, 1H), 2.70 (td, 1H), 2.46 (m, 1H), 2.36-2.23 (m, 2H), 2.23-2.00 (m, 3H), 1.83-1.67 (m, 2H), 1.06 (m, 1H), 0.11 (m, 1H).

### Example 107 (1r,1′aR*,4r,6′aS*)-4-(3-chloroanilino)-1′a,6′a-dihydro-1′H-spiro[cyclohexane-1,6′-cyclopropa[a]indene]-4-carboxylic acid, enantiomer 1

and

### Example 108 (1r,1′aR*,4r,6′aS*)-4-(3-chloroanilino)-1′a,6′a-dihydro-1′H-spiro[cyclohexane-1,6′-cyclopropa[a]indene]-4-carboxylic acid, enantiomer 2

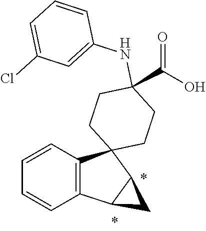 [SMILES: O=C(O)[C@]1(Nc2cccc(Cl)c2)CC[C@@]2(CC1)c1ccccc1[C@@H]1C[C@@H]12]

Using General procedure 10 and Example 107C as the appropriate ketone a racemic mixture of diastereoisomers was obtained. The diastereoisomers were separated via flash chromatography using heptane and EtOAc as eluents. The enantiomers of the firstly eluting diastereoisomer were separated by chiral chromatography. Column: AD, 50×500 mm, 20 μm, eluents: EtOH+0.05% HCOOH. The enantiomer eluting earlier was collected and purified via prep RP-HPLC using 25 mM aq. NH₄HCO₃ solution and MeCN as eluents resulting Example 107. HRMS calculated for C₂₂H₂₂NO₂Cl: 367.1339; found: 368.1412 (M+H).

The enantiomer eluting later was collected as Example 108. HRMS calculated for C₂₂H₂₂NO₂Cl: 367.1339; found: 368.1416 (M+H).

### Example 109 (1r,4r)-4-(3-chloroanilino)-2′-[(1R*,2S*)-2-(phenoxymethyl)cyclopropyl]-2′,3′-dihydrospiro[cyclohexane-1,1′-indene]-4-carboxylic acid, diastereoisomer 1 enantiomer 1

and

### Example 110 (1r,4r)-4-(3-chloroanilino)-2′-[(1R*,2S*)-2-(phenoxymethyl)cyclopropyl]-2′,3′-dihydrospiro[cyclohexane-1,1′-indene]-4-carboxylic acid, diastereoisomer 2 enantiomer 1

and

### Example 111 (1r,4r)-4-(3-chloroanilino)-2′-[(1R*,2S*)-2-(phenoxymethyl)cyclopropyl]-2′,3′-dihydrospiro[cyclohexane-1,1′-indene]-4-carboxylic acid, diastereoisomer 1 enantiomer 2

and

### Example 112 (1r,4r)-4-(3-chloroanilino)-2′-[(1R*,2S*)-2-(phenoxymethyl)cyclopropyl]-2′,3′-dihydrospiro[cyclohexane-1,1′-indene]-4-carboxylic acid, diastereoisomer 2 enantiomer 2

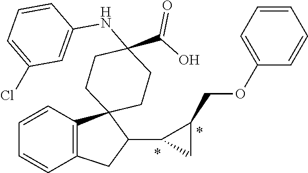

Using General procedure 32 and Example 103C as the appropriate indane and phenol as the appropriate alcohol, a racemic distereoisomer was obtained. The enantiomers were separated by chiral chromatography. Column: AD, 50×500 mm, 20 μm, eluents: heptane/EtOH+0.05% HCOOH. The enantiomer eluting earlier was collected as Example 109. HRMS calculated for C₃₁H₃₂NO₃Cl: 501.2071; found: 502.2167 (M+H).

The enantiomer eluting later was collected as Example 111. HRMS calculated for C₃₁H₃₂NO₃Cl: 501.2071; found: 502.2172 (M+H).

Using General procedure 32 and Example 103D as the appropriate indane and phenol as the appropriate alcohol, a racemic distereoisomer was obtained. The enantiomers were separated by chiral chromatography. Column: AD, 50×500 mm, 20 μm, eluents: heptane/EtOH+0.05% HCOOH. The enantiomer eluting earlier was collected as Example 110. HRMS calculated for C₃₁H₃₂NO₃Cl: 501.2071; found: 502.2144 (M+H).

The enantiomer eluting later was collected as Example 112. HRMS calculated for C₃₁H₃₂NO₃Cl: 501.2071; found: 502.2165 (M+H).

### Example 113 (1r,4r)-4-(3-chloroanilino)-2′-[(1R*,2S*)-2-{[(pyridin-3-yl)oxy]methyl}cyclopropyl]-2′,3′-dihydrospiro[cyclohexane-1,1′-indene]-4-carboxylic acid, diastereoisomer 1 enantiomer 2

and

### Example 114 (1r,4r)-4-(3-chloroanilino)-2′-[(1R*,2S*)-2-{[(pyridin-3-yl)oxy]methyl}cyclopropyl]-2′,3′-dihydrospiro[cyclohexane-1,1′-indene]-4-carboxylic acid, diastereoisomer 2 enantiomer 2

and

### Example 115 (1r,4r)-4-(3-chloroanilino)-2′-[(1R*,2S*)-2-{[(pyridin-3-yl)oxy]methyl}cyclopropyl]-2′,3′-dihydrospiro[cyclohexane-1,1′-indene]-4-carboxylic acid, diastereoisomer 1 enantiomer 1

and

### Example 116 (1r,4r)-4-(3-chloroanilino)-2′-[(1R*,2S*)-2-{[(pyridin-3-yl)oxy]methyl}cyclopropyl]-2′,3′-dihydrospiro[cyclohexane-1,1′-indene]-4-carboxylic acid, diastereoisomer 2 enantiomer 1

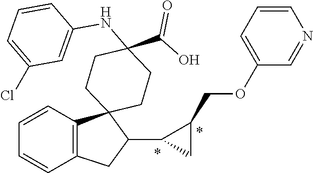

Using General procedure 32 and Example 103C as the appropriate indane and pyridin-3-ol as the appropriate alcohol, a racemic distereoisomer was obtained. The enantiomers were separated by chiral chromatography. Column: AD, 50×500 mm, 20 μm, eluents: heptane/EtOH+0.05% HCOOH. The enantiomer eluting later was collected as Example 113. HRMS calculated for C₃₀H₃₁N₂O₃Cl: 502.2023; found: 503.2087 (M+H).

The enantiomer eluting earlier was collected as Example 115. HRMS calculated for C₃₀H₃₁N₂O₃Cl: 502.2023; found: 503.2088 (M+H).

Using General procedure 32 and Example 103D as the appropriate indane and pyridin-3-ol as the appropriate alcohol, a racemic distereoisomer was obtained. The enantiomers were separated by chiral chromatography. Column: AD, 50×500 mm, 20 μm, eluents: heptane/EtOH+0.05% HCOOH. The enantiomer eluting later was collected as Example 114. HRMS calculated for C₃₀H₃₁N₂O₃Cl: 502.2023; found: 503.2087 (M+H).

The enantiomer eluting earlier was collected as Example 116. HRMS calculated for C₃₀H₃₁N₂O₃Cl: 502.2023; found: 503.2093 (M+H).

### Example 117

### Example 117A dispiro[[1,3]dioxolane-2,1′-cyclohexane-4′,9″-fluorene]

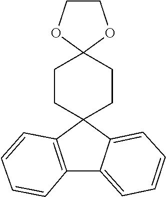 [SMILES: c1ccc2c(c1)-c1ccccc1C21CCC2(CC1)OCCO2]

Using General procedure 8a and 9H-fluorene as the appropriate indene Example 117A was obtained. ¹H NMR (400 MHz, DMSO-d₆) δ ppm: 7.89-7.86 (m, 2H), 7.64-7.61 (m, 2H), 7.39 (td, J=7.4, 1.2 Hz, 2H), 7.33 (td, J=7.4, 1.4 Hz, 2H), 4.01 (s, 4H), 2.06-1.99 (m, 4H), 1.87-1.78 (m, 4H).

### Example 117B spiro[cyclohexane-1,9′-fluoren]-4-one

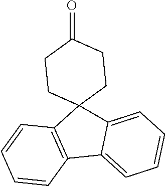 [SMILES: O=C1CCC2(CC1)c1ccccc1-c1ccccc12]

Using General procedure 9 and Example 117A as appropriate ketal Example 117B was obtained. ¹H NMR (400 MHz, DMSO-d₆) δ ppm: 7.92-7.89 (m, 2H), 7.86-7.83 (m, 2H), 7.42 (td, J=7.4, 1.1 Hz, 2H), 7.34 (td, J=7.5, 1.3 Hz, 2H), 2.74 (t, J=6.9 Hz, 4H), 2.11 (t, J=7.0 Hz, 4H).

### Example 117 4-[(3-bromophenyl)amino]spiro[cyclohexane-1,9′-fluorene]-4-carboxylic acid

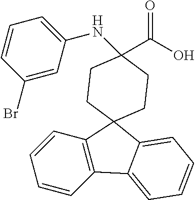 [SMILES: O=C(O)C1(Nc2cccc(Br)c2)CCC2(CC1)c1ccccc1-c1ccccc12]

A solution of Example 117B (47 mg, 0.19 mmol, 1 eq) in THE (1 mL) was treated as described in General procedure 10 using 3-bromoaniline (21 μL, 0.19 mmol, 1 eq) as the appropriate aniline to afford Example 117 as a cream solid (40.8 mg, 0.08 mmol, 44%). LRMS calculated for C₂₅H₂₂NO₂Br: 447; found: 448 (M+H). ¹H NMR (400 MHz, DMSO-d₆) δ ppm: 12.80 (br s, 1H), 7.89 (dd, J=7.4, 1.3 Hz, 1H), 7.88-7.83 (m, 1H), 7.72 (d, J=7.4 Hz, 1H), 7.60-7.54 (m, 1H), 7.43-7.33 (m, 4H), 7.05 (t, J=8.1 Hz, 1H), 6.89 (t, J=2.1 Hz, 1H), 6.72 (ddd, J=7.8, 1.8, 0.7 Hz, 1H), 6.66 (ddd, J=8.3, 2.3, 0.9 Hz, 1H), 6.47 (br s, 1H), 2.53-2.41 (m, 2H), 2.27-2.14 (m, 4H), 1.46-1.37 (m, 2H).

### Example 118 4-(3-chloroanilino)spiro[cyclohexane-1,9′-fluorene]-4-carboxylic acid

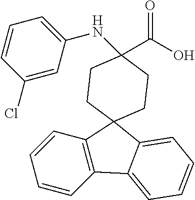 [SMILES: O=C(O)C1(Nc2cccc(Cl)c2)CCC2(CC1)c1ccccc1-c1ccccc12]

Using General procedure 10 and Example 117B as the appropriate ketone and 3-chloroaniline as the appropriate aniline Example 118 was obtained. ¹H NMR (500 MHz, DMSO-d₆) δ ppm: 7.87 (dm, 1H), 7.83 (m, 1H), 7.75 (dm, 1H), 7.58 (m, 1H), 7.42-7.29 (m, 4H), 7.02 (t, 1H), 6.75 (t, 1H), 6.66 (dm, 1H), 6.48 (dm, 1H), 6.28 (br s, 1H), 2.47/2.13 (m+m, 4H), 2.10/1.43 (m+m, 4H). HRMS calculated for C₂₅H₂₂NO₂Cl: 403.1339; found: 404.1422 (M+H).

### Example 119

### Example 119A 1-(methoxymethylidene)-1,2,3,4-tetrahydronaphthalene

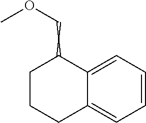

To a stirred suspension of K^{t}OBu (1.77 g, 15.76 mmol, 2.3 eq) in 1,4-dioxane (25 mL) was added (methoxymethyl)triphenylphosphonium chloride (5.40 g, 15.76 mmol, 2.3 eq) under N₂ and the mixture was stirred at rt for 2 h. A solution of 1,2,3,4-tetrahydronaphthalen-1-one (1.0 g, 6.84 mmol, 1 eq) in 1,4-dioxane (10 mL) was added and the mixture was stirred at rt for 18 h. The reaction mixture was diluted with water and extracted with EtOAc. The combined organic phase was washed with water, brine, dried (MgSO₄) and concentrated in vacuo. Purification by flash column chromatography (50 g silica cartridge) eluting with a gradient of 0-5% Et₂O in heptane afforded a mixture of E/Z isomers Example 119A (1.19 g, 6.84 mmol, 100%). ¹H NMR (400 MHz, DMSO-d₆) δ ppm: 8.05-8.01/7.47-7.43 (m, 1H), 7.12-6.97 (m, 3H), 6.87/6.24 (t, 1H), 3.70/3.69 (s, 3H), 2.77/2.66 (t, 2H), 2.43-2.38/2.28-2.23 (m, 2H), 1.79-1.63 (m, 2H).

### Example 119B 3′,4′-dihydro-2′H-spiro[cyclohex-2-ene-1,1′-naphthalen]-4-one

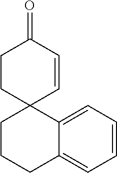 [SMILES: O=C1C=CC2(CCCc3ccccc32)CC1]

To a solution of Example 119A (1.3 g, 7.46 mmol, 1 eq) in toluene (15 mL) was added MVK (684 μL, 8.2 mmol, 1.1 eq) and PTSA (142 mg, 0.75 mmol, 0.1 eq) at rt. The mixture was stirred at 85° C. for 18 h under N₂ and allowed to cool to rt and then poured into sat. aq. NaHCO₃ solution. The organic layer was separated and the aq. layer was extracted with EtOAc. The combined organic phase was washed with water, brine, dried (MgSO₄) and concentrated in vacuo. Purification by flash column chromatography (20 g silica cartridge) eluting with a gradient of 0-25% Et₂O in heptane afforded Example 119B as a racemic yellow solid (640 mg, 3.01 mmol, 40%). LRMS calculated for C₁₅H₁₆O: 212; found 213 (M+H). ¹H NMR (400 MHz, DMSO-d₆) δ ppm: 7.19-7.06 (m, 4H), 6.79 (dd, J=10.1, 1.7 Hz, 1H), 5.99 (dd, J=10.1, 0.7 Hz, 1H), 2.86-2.74 (m, 2H), 2.63-2.52 (m, 1H), 2.32-2.16 (m, 2H), 2.11-2.02 (m, 2H), 1.90-1.78 (m, 2H), 1.74-1.64 (m, 1H).

### Example 119C 3′,4′-dihydro-2′H-spiro[cyclohexane-1,1′-naphthalen]-4-one

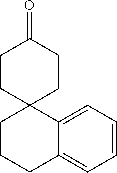 [SMILES: O=C1CCC2(CCCc3ccccc32)CC1]

To a solution of Example 119B (565 mg, 2.66 mmol, 1 eq) in AcOH (25 mL) was added 10% Pd/C (57 mg). The flask was evacuated and backfilled with N₂ (×3), then evacuated and filled with H₂. The reaction mixture was shaken at rt for 18 h. 10% Pd/C (283 mg) was added and the flask was evacuated and backfilled with N₂ (×3), then evacuated and filled with H₂. The reaction mixture was shaken at rt for 16 h after which time no more conversion was observed. The reaction mixture was filtered and poured onto cold 10% aq. NH₃ solution (100 mL) and stirring continued for 10 min. The mixture was extracted with EtOAc and the organic phase was separated, washed with brine, dried (MgSO₄) and concentrated in vacuo to afford Example 119C as a white solid (477 mg, 2.23 mmol, 84%). ¹H NMR (400 MHz, DMSO-d₆) δ ppm: 7.44 (dd, J=7.6, 1.3 Hz, 1H), 7.15-7.03 (m, 3H), 2.76 (t, J=6.3 Hz, 2H), 2.66-2.56 (m, 2H), 2.24-2.13 (m, 4H), 2.05-1.99 (m, 2H), 1.87-1.73 (m, 4H).

### Example 119 (1r,4s)-4-(3-bromoanilino)-3′,4′-dihydro-2′H-spiro[cyclohexane-1,1′-naphthalene]-4-carboxylic acid

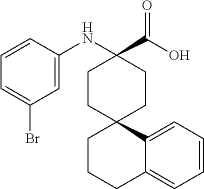 [SMILES: O=C(O)[C@]1(Nc2cccc(Br)c2)CC[C@@]2(CCCc3ccccc32)CC1]

Example 119C (100 mg, 0.47 mmol, 1 eq) was treated according to General Procedure 10 using 3-bromoaniline (51 μL, 0.47 mmol, 1 eq) to obtain a mixture of diastereoisomers.

Purification of the crude product by flash column chromatography (5 g silica cartridge) eluting with a gradient of 0-10% EtOAc in DCM afforded the major diastereoisomer Example 119 as a yellow foam (26.8 mg, 0.06 mmol, 14%). LRMS calculated for C₂₂H₂₄BrNO₂: 413; found: 414 (M+H). ¹H NMR (400 MHz, DMSO-d₆) δ ppm: 7.25-7.20 (m, 1H), 7.18-7.13 (m, 1H), 7.10-6.97 (m, 3H), 6.82 (t, J=2.1 Hz, 1H), 6.71-6.67 (m, 1H), 6.65-6.61 (m, 1H), 2.74-2.67 (m, 2H), 2.38-2.29 (m, 2H), 2.07-1.96 (m, 2H), 1.85-1.77 (m, 2H), 1.73-1.62 (m, 4H), 1.60-1.50 (m, 2H).

### Example 120 (1r,4s)-4-[(3,5-dichlorophenyl)amino]-3′,4′-dihydro-2′H-spiro[cyclohexane-1,1′-naphthalene]-4-carboxylic acid

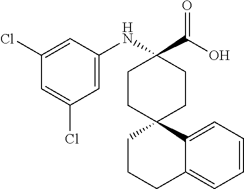 [SMILES: O=C(O)[C@]1(Nc2cc(Cl)cc(Cl)c2)CC[C@@]2(CCCc3ccccc32)CC1]

Example 119C (100 mg, 0.47 mmol) was treated as described in General procedure 10 using 3,5-dichloroaniline (76 mg, 0.47 mmol) to obtain a mixture of diastereoisomers. Purification by automated flash chromatography (CombiFlash Rf, 12 g RediSep™ silica cartridge) eluting with a gradient of 0-10% EtOAc in DCM followed by preparative HPLC at pH 9 and then pH 4 afforded the major diastereoisomer Example 120 as a white solid (4.4 mg, 0.01 mmol, 2%). LRMS calculated for C₂₂H₂₃NO₂Cl₂: 403; found: 404 (M+H). ¹H NMR (400 MHz, DMSO-d6) δ ppm: 12.98 (br s, 1H), 7.23 (d, J=7.8 Hz, 1H), 7.15 (td, J=7.9, 7.4, 1.9 Hz, 1H), 7.08-7.00 (m, 2H), 6.64 (s, 3H), 6.52 (br s, 1H), 2.74-2.67 (m, 2H), 2.36-2.27 (m, 2H), 2.08-1.96 (m, 2H), 1.84-1.77 (m, 2H), 1.73-1.60 (m, 4H), 1.59-1.50 (m, 2H).

### Example 121

### Example 121A dispiro[[1,3]dioxolane-2,1′-cyclohexane-4′,1″-indene]

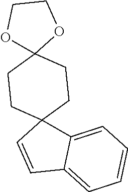 [SMILES: C1=CC2(CCC3(CC2)OCCO3)c2ccccc21]

To a solution of indene (1.5 g, 12.91 mmol, 1 eq) and Preparation 1a (4.11 g, 12.91 mmol, 1 eq) in anhydrous DMF (48 mL), cooled to 0° C. under a N₂ atmosphere, was added NaH (60% dispersion; 1.08 g, 27.12 mmol, 2.1 eq). The mixture was allowed to warm to rt and stirred for 16 h. The reaction was quenched with sat. aq. NH₄Cl solution, stirred for 15 min and then concentrated in vacuo. The crude material was diluted with sat. aq. NaHCO₃ solution and extracted with DCM. The combined organic extracts were washed with brine, dried (MgSO₄) and concentrated in vacuo. Purification by flash chromatography (50 g silica cartridge) eluting with a stepped gradient of 0-10% EtOAc in heptane afforded Example 121A as a yellow solid (2.15 g, 8.86 mmol, 69%). ¹H NMR (400 MHz, DMSO-d₆) δ ppm: 7.35-7.31 (m, 2H), 7.24-7.14 (m, 2H), 7.01 (d, J=5.6 Hz, 1H), 6.79 (d, J=5.6 Hz, 1H), 3.99-3.90 (m, 4H), 2.14-2.03 (m, 2H), 1.90-1.79 (m, 4H), 1.26-1.18 (m, 2H).

### Example 121B rac-(1″aR,6″aS)-1″a,6″a-dihydrodispiro[[1,3]dioxolane-2,1′-cyclohexane-4′,6″-indeno[1,2-b]oxirene]

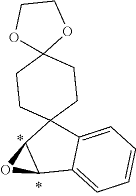 [SMILES: c1ccc2c(c1)[C@H]1O[C@H]1C21CCC2(CC1)OCCO2]

To a suspension of Example 121A (366 mg, 1.51 mmol, 1 eq) in DCM (5 mL) cooled to 0° C., was added mCPBA (0.79 mL, 1.96 mmol, 1.3 eq). The reaction was allowed to warm to rt and stirred for 18 h. The mixture was diluted with DCM, washed with sat. aq. Na₂S₂O₃ solution and the organic phase dried (MgSO₄) and concentrated in vacuo. Purification by automated flash chromatography (CombiFlash Rf, 24 g RediSep™ silica cartridge) eluting with a gradient of 0-35% EtOAc in heptane afforded a racemic diastereoisomer, Example 121B as a white solid (177 mg, 0.69 mmol, 45%). ¹H NMR (400 MHz, DMSO-d₆) δ ppm: 7.53-7.49 (m, 1H), 7.30-7.25 (m, 1H), 7.21-7.16 (m, 2H), 4.35 (d, J=2.8 Hz, 1H), 4.25 (d, J=2.9 Hz, 1H), 3.98-3.90 (m, 4H), 2.03-1.75 (m, 5H), 1.73-1.60 (m, 2H), 1.48-1.40 (m, 1H).

### Example 121C 2″,3″-dihydrodispiro[[1,3]dioxolane-2,1′-cyclohexane-4′,1″-inden]-2″-ol

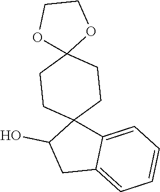 [SMILES: OC1Cc2ccccc2C12CCC1(CC2)OCCO1]

To a solution of Example 121B (177 mg, 0.69 mmol, 1 eq) in 1,4-dioxane (20 mL) was added Pd/C (50 mg) and NH₄HCO₃ (691 mg, 10.96 mmol, 16 eq) and the mixture was stirred at 80° C. for 2 h. The reaction was cooled to rt, filtered through celite, washed with DCM and concentrated in vacuo. Purification by automated flash chromatography (CombiFlash Rf, 25 g RediSep™ silica cartridge) eluting with a gradient of 0-50% EtOAc in heptane afforded a racemic mixture, Example 121C as a white solid (122 mg, 0.47 mmol, 68%). LRMS calculated for C₁₆H₂₀O₃: 260; found: 261 (M+H). ¹H NMR (400 MHz, DMSO-d₆) δ ppm: 7.20-7.09 (m, 4H), 4.83 (d, J=5.0 Hz, 1H), 4.32-4.28 (m, 1H), 3.95-3.85 (m, 4H), 3.16 (ddd, J=16.3, 5.5, 1.1 Hz, 1H), 2.70 (dd, 1H), 2.00-1.93 (m, 1H), 1.84-1.42 (m, 7H).

### Example 121D 2″-[(prop-2-en-1-yl)oxy]-2″,3″-dihydrodispiro[[1,3]dioxolane-2,1′-cyclohexane-4′,1″-indene]

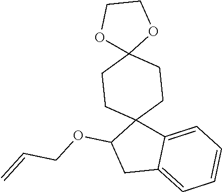 [SMILES: C=CCOC1Cc2ccccc2C12CCC1(CC2)OCCO1]

To a solution of Example 121C (81 mg, 0.31 mmol, 1 eq) in DMF (3 mL), cooled to 0° C., was added NaH (60% dispersion; 19 mg, 0.47 mmol, 1.5 eq) and the mixture stirred for 20 min at 0° C. Allyl bromide (40 μL, 0.47 mmol, 1.5 eq) was added and the mixture was stirred at rt for 18 h. The reaction was quenched by the careful addition of brine and then extracted with EtOAc. The combined organic extracts were washed with brine, dried (MgSO₄) and concentrated in vacuo. Purification by automated flash chromatography (CombiFlash Rf, 24 g RediSep™ silica cartridge) eluting with a gradient of 0-100% EtOAc in heptane afforded a racemic mixture, Example 121D as a colourless oil (62 mg, 0.21 mmol, 66%). LRMS calculated for C₁₉H₂₄O₃: 300; found: 301 (M+H). ¹H NMR (400 MHz, DMSO-d₆) δ ppm: 7.22-7.11 (m, 4H), 5.95-5.85 (m, 1H), 5.24 (dq, J=17.3, 1.8 Hz, 1H), 5.14-5.09 (m, 1H), 4.15-4.08 (m, 2H), 3.96-3.85 (m, 5H), 3.10 (dd, J=16.6, 5.2 Hz, 1H), 2.90 (dd, J=16.6, 2.4 Hz, 1H), 2.04-1.95 (m, 1H), 1.87-1.45 (m, 7H).

### Example 121E 2′-[(prop-2-en-1-yl)oxy]-2′,3′-dihydrospiro[cyclohexane-1,1′-inden]-4-one

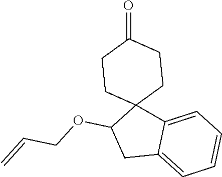 [SMILES: C=CCOC1Cc2ccccc2C12CCC(=O)CC2]

A solution of Example 121D (62 mg, 0.21 mmol, 1 eq) in acetone (4 mL) was treated with 2 M aq. HCl solution (1 mL, 2 mmol, 10 eq) and then stirred at rt for 3 h. The reaction was neutralised with sat. aq. NaHCO₃ solution and partitioned between EtOAc and water. The organic phase was washed with brine, dried (MgSO₄) and concentrated in vacuo to afford a racemic mixture, Example 121E as a solid (49 mg, 0.19 mmol, 93%). ¹H NMR (400 MHz, DMSO-d₆) δ ppm: 7.28-7.14 (m, 4H), 5.99-5.89 (m, 1H), 5.24 (dq, J=17.2, 1.8 Hz, 1H), 5.15-5.10 (m, 1H), 4.36 (dd, J=5.4, 3.2 Hz, 1H), 4.17 (ddt, J=13.0, 5.2, 1.5 Hz, 1H), 4.02 (ddt, J=13.0, 5.4, 1.6 Hz, 1H), 3.20 (dd, J=16.4, 5.4 Hz, 1H), 2.97 (dd, J=16.4, 3.2 Hz, 1H), 2.74-2.55 (m, 2H), 2.33-2.20 (m, 3H), 2.06-1.96 (m, 1H), 1.95-1.77 (m, 2H).

### Example 121 (1s,4s)-4-(3-bromoanilino)-2′-[(prop-2-en-1-yl)oxy]-2′,3′-dihydrospiro[cyclohexane-1,1′-indene]-4-carboxylic acid

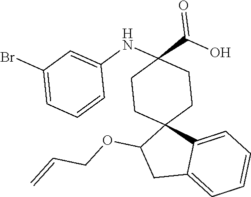

Example 121E (49 mg, 0.19 mmol, 1 eq) was treated as described in General procedure 10 using 3-bromoaniline (21 μL, 0.19 mmol, 1 eq). A mixture of diastereoisomers was formed. The mixture was diluted with water, acidified with 2 M aq. HCl solution and extracted with DCM. The combined organic extracts were dried (MgSO₄) and concentrated in vacuo and purified by automated flash chromatography (CombiFlash Rf, 12 g RediSep™ silica cartridge) eluting with a gradient of 0-100% EtOAc in heptane. The racemic diastereoisomer eluting earlier was collected as Example 121, a beige solid (18.7 mg, 0.04 mmol, 21%). LRMS calculated for C₂₄H₂₆NO₃Br: 455; found: 456 (M+H). ¹H NMR (400 MHz, DMSO-d₆) δ ppm: 12.67 (br s, 1H), 7.23-7.13 (m, 4H), 7.00 (t, J=8.0 Hz, 1H), 6.81 (t, J=2.1 Hz, 1H), 6.69 (dd, J=7.6, 1.7 Hz, 1H), 6.63-6.59 (m, 1H), 5.97-5.86 (m, 1H), 5.26 (dq, J=17.3, 1.9 Hz, 1H), 5.12 (dq, J=10.5, 1.6 Hz, 1H), 4.17-4.10 (m, 1H), 4.03-3.92 (m, 2H), 3.09 (dd, J=16.4, 5.4 Hz, 1H), 2.87 (dd, J=16.5, 3.2 Hz, 1H), 2.46-2.35 (m, 1H), 2.30-2.20 (m, 1H), 2.10-2.01 (m, 1H), 1.88-1.76 (m, 2H), 1.71-1.52 (m, 3H).

### Example 122 (1s,4s)-4-(3-chloroanilino)-2′-[(prop-2-en-1-yl)oxy]-2′,3′-dihydrospiro[cyclohexane-1,1′-indene]-4-carboxylic acid

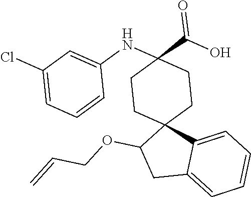

Example 121E (1.17 g, 4.56 mmol, 1 eq) was treated as described in General procedure 10 using 3-chloroaniline (478 μL, 4.56 mmol, 1 eq). A mixture of diastereoisomers was formed. The mixture was diluted with water, acidified with 2 M aq. HCl solution and extracted with DCM. The organic phase was dried (MgSO₄), concentrated in vacuo and purified by automated flash chromatography (CombiFlash Rf, 80 g RediSep™ silica cartridge) eluting with a gradient of 0-100% EtOAc in heptane. The racemic diastereoisomer eluting earlier was collected as Example 122, a beige solid (480 mg, 1.17 mmol, 26%). LRMS calculated for C₂₄H₂₆NO₃Cl: 411; found: 412 (M+H). ¹H NMR (400 MHz, DMSO-d₆) δ ppm: 7.23-7.13 (m, 4H), 7.06 (t, J=8.1 Hz, 1H), 6.65 (t, J=2.1 Hz, 1H), 6.60-6.53 (m, 2H), 5.97-5.86 (m, 1H), 5.26 (dq, J=17.2, 1.8 Hz, 1H), 5.12 (dq, J=10.5, 1.6 Hz, 1H), 4.13 (ddt, J=13.3, 5.0, 1.6 Hz, 1H), 4.03-3.92 (m, 2H), 3.10 (dd, J=16.3, 5.3 Hz, 1H), 2.87 (dd, J=16.7, 3.2 Hz, 1H), 2.45-2.36 (m, 1H), 2.30-2.20 (m, 1H), 2.10-2.01 (m, 1H), 1.88-1.76 (m, 2H), 1.71-1.51 (m, 3H).

### Example 123

### Example 123A 2″,4″,7″-trimethyldispiro[[1,3]dioxolane-2,1′-cyclohexane-4′,1″-indene]

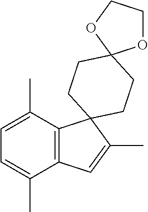 [SMILES: CC1=Cc2c(C)ccc(C)c2C12CCC1(CC2)OCCO1]

Using General procedure 8a with Preparation 1a and 2,4,7-trimethyl-1H-indene as the appropriate indene, Example 123A was obtained as a light brown solid (304 mg, 1.07 mmol, 67%). ¹H NMR (400 MHz, CDCl₃) δ ppm: 6.89 (d, J=7.7 Hz, 1H), 6.80 (d, J=7.7 Hz, 1H), 6.36 (s, 1H), 4.08-3.99 (m, 4H), 2.79 (td, J=14.3, 5.0 Hz, 2H), 2.53 (s, 3H), 2.30 (s, 6H), 2.13 (td, J=14.0, 5.1 Hz, 2H), 1.84-1.76 (m, 2H), 1.49-1.40 (m, 2H).

### Example 123B 2″,4″,7″-trimethyl-2″,3″-dihydrodispiro[[1,3]dioxolane-2,1′-cyclohexane-4′,1″-indene]

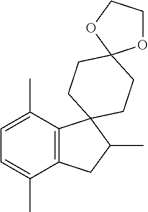 [SMILES: Cc1ccc(C)c2c1CC(C)C21CCC2(CC1)OCCO2]

Using General procedure 19 and Example 123A as the appropriate indene afforded a racemate, Example 123B as a colourless oil (150 mg, 0.52 mmol, 99%). ¹H NMR (400 MHz, CDCl₃) δ ppm: 6.87 (d, J=7.7 Hz, 1H), 6.83 (d, J=7.7 Hz, 1H), 4.06-3.93 (m, 4H), 3.04-2.95 (m, 1H), 2.67-2.53 (m, 2H), 2.44-2.35 (m, 4H), 2.19 (s, 3H), 1.93-1.80 (m, 3H), 1.78-1.61 (m, 3H), 1.59-1.50 (m, 1H), 0.93 (d, J=7.0 Hz, 3H).

### Example 123C 2′,4′,7′-trimethyl-2′,3′-dihydrospiro[cyclohexane-1,1′-inden]-4-one

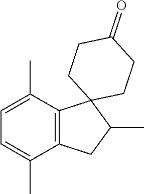 [SMILES: Cc1ccc(C)c2c1CC(C)C21CCC(=O)CC1]

Using General Procedure 9 and Example 123B as the appropriate ketal afforded a racemate, Example 123C as a white solid (120 mg, 0.50 mmol, 94%). ¹H NMR (400 MHz, CDCl₃) δ ppm: 6.90 (d, J=7.6 Hz, 1H), 6.85 (d, J=7.6 Hz, 1H), 3.07 (dd, J=15.9, 6.9 Hz, 1H), 2.80-2.71 (m, 1H), 2.71-2.58 (m, 2H), 2.54-2.37 (m, 4H), 2.35 (s, 3H), 2.26-2.16 (m, 4H), 2.03 (td, J=13.6, 5.0 Hz, 1H), 1.96-1.86 (m, 1H), 1.05 (d, J=6.9 Hz, 3H).

### Example 123 4-(3-bromoanilino)-2′,4′,7′-trimethyl-2′,3′-dihydrospiro[cyclohexane-1,1′-indene]-4-carboxylic acid

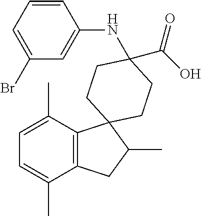 [SMILES: Cc1ccc(C)c2c1CC(C)C21CCC(Nc2cccc(Br)c2)(C(=O)O)CC1]

Using General procedure 10 and Example 123C as the appropriate ketone and 3-bromoaniline as the appropriate aniline, a racemic mixture of diastereoisomers, Example 123 was obtained as a white solid (8 mg, 0.02 mmol, 11%). LRMS calculated for C₂₄H₂₈BrNO₂: 441; found: 442 (M+H). ¹H NMR (400 MHz, DMSO-d₆) δ ppm: 12.80 (br s, 1H), 6.99 (t, J=8.1 Hz, 1H), 6.85-6.80 (m, 2H), 6.78 (d, J=7.6 Hz, 1H), 6.71-6.66 (m, 1H), 6.65-6.60 (m, 1H), 6.15 (br s, 1H), 2.97 (dd, J=16.0, 7.0 Hz, 1H), 2.62-2.43 (m, 3H), 2.40-2.31 (m, 2H), 2.29 (s, 3H), 2.13 (s, 3H), 1.91-1.80 (m, 1H), 1.78-1.59 (m, 2H), 1.59-1.49 (m, 1H), 1.48-1.39 (m, 1H), 0.86 (d, J=6.8 Hz, 3H).

### Example 124

### Example 124A 6-chloro-2-methyl-2,3-dihydro-1H-inden-1-ol

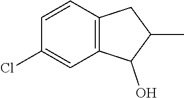

To a stirred solution of 6-chloro-2-methyl-2,3-dihydro-1H-inden-1-one (1.2 g, 6.67 mmol, 1 eq) in MeOH (20 mL) at 0° C. was added NaBH₄ (254 mg, 6.7 mmol, 1 eq) in portions. After addition, the reaction mixture was stirred at rt for 1 h and then quenched by the addition of water (5 mL). MeOH was removed in vacuo and the residue was diluted with water and extracted with EtOAc. The combined organic extracts were washed with brine, dried (MgSO₄) and concentrated in vacuo to afford Example 124A as a racemic mixture of diastereoisomers, isolated as a white solid (0.90 g, 4.94 mmol, 74%). ¹H NMR (400 MHz, CDCl₃) δ ppm: 7.40-7.33 (m, 1H), 7.24-7.08 (m, 2H), 5.03-4.97/4.74-4.66 (m, 1H), 3.11-3.01/2.96-2.88 (m, 1H), 2.68-2.20 (m, 2H), 1.81-1.73/1.55-1.48 (m, 1H), 1.26/1.12 (d, J=6.7 Hz, 3H).

### Example 124B 5-chloro-2-methyl-1H-indene

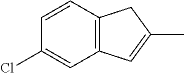

Using General procedure 7 and Example 124A as the appropriate indane, Example 124B was obtained as a white solid (0.70 g, 4.27 mmol, 86%). ¹H NMR (400 MHz, CDCl₃) δ ppm: 7.25 (d, J=7.9 Hz, 1H), 7.20 (d, J=1.9 Hz, 1H), 7.05 (dd, J=7.9, 1.9 Hz, 1H), 6.42 (s, 1H), 3.26 (s, 2H), 2.15 (s, 3H).

### Example 124C 5″-chloro-2″-methyldispiro[[1,3]dioxolane-2,1′-cyclohexane-4′,1″-indene]

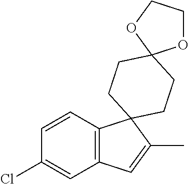 [SMILES: CC1=Cc2cc(Cl)ccc2C12CCC1(CC2)OCCO1]

Using General procedure 8a with Preparation 1a and Example 124B as the appropriate indene, a mixture of regioisomers was obtained. They were separated by flash chromatography using heptane and EtOAc as eluents. The regioisomer eluting later was collected as Example 124C, isolated as a brown solid (250 mg, 0.86 mmol, 20%). ¹H NMR (400 MHz, CDCl₃) δ ppm: 7.57 (d, J=8.1 Hz, 1H), 7.20 (d, J=2.0 Hz, 1H), 7.04 (dd, J=8.1, 2.0 Hz, 1H), 6.33 (s, 1H), 4.04 (s, 4H), 2.20 (td, J=13.5, 4.0 Hz, 2H), 2.08 (td, J=13.4, 3.7 Hz, 2H), 1.98 (s, 3H), 1.92-1.84 (m, 2H), 1.27-1.17 (m, 2H).

### Example 124D 5′-chloro-2′-methylspiro[cyclohexane-1,1′-inden]-4-one

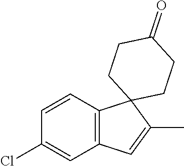 [SMILES: CC1=Cc2cc(Cl)ccc2C12CCC(=O)CC2]

Using General procedure 9 and Example 124C as the appropriate ketal, Example 124D was obtained as a brown solid (80 mg, 0.33 mmol, 94%). LRMS calculated for C₁₅H₁₅ClO: 246; found: 247 (M+H).

### Example 124 4-(3-bromoanilino)-5′-chloro-2′-methylspiro[cyclohexane-1,1′-indene]-4-carboxylic acid

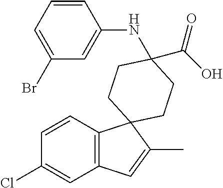 [SMILES: CC1=Cc2cc(Cl)ccc2C12CCC(Nc1cccc(Br)c1)(C(=O)O)CC2]

Using General procedure 10 and Example 124D as the appropriate ketone and 3-bromoaniline as the appropriate aniline, a mixture of diastereoisomers, Example 124 was obtained as a white solid (8 mg, 0.018 mmol, 9%). LRMS calculated for C₂₂H₂₁BrClNO₂: 445; found: 446 (M+H). ¹H NMR (400 MHz, DMSO-d₆) δ ppm: 7.62 (d, J=8.1 Hz, 1H), 7.31 (d, J=2.1 Hz, 1H), 7.14 (dd, J=8.1, 2.1 Hz, 1H), 7.03 (t, J=8.1 Hz, 1H), 6.86 (t, J=2.0 Hz, 1H), 6.75-6.63 (m, 2H), 6.46-6.41 (m, 1H), 2.52-2.43 (m, 2H), 2.12-1.82 (m, 7H), 1.34-1.23 (m, 2H).

### Example 125

### Example 125A 2,4-dimethyl-2,3-dihydro-1H-inden-1-ol

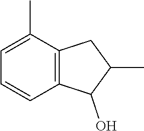

To a stirred solution of 2,4-dimethyl-2,3-dihydro-1H-inden-1-one (3.30 g, 20.6 mmol, 1 eq) in MeOH (200 mL) at 0° C. was added NaBH₄ (0.94 g, 24.8 mmol, 1.2 eq) in portions. After addition, the reaction mixture was stirred at rt for 1 h and then quenched by the addition of sat. aq. NH₄Cl solution (5 mL). The mixture was extracted with EtOAc and the combined organic extracts were washed with brine, dried (MgSO₄) and concentrated in vacuo to afford Example 125A as a racemic mixture of diastereoisomers, isolated as a yellow solid (3.24 g, 20 mmol, 97%). ¹H NMR (400 MHz, CDCl₃) δ ppm: 7.28-7.05 (m, 3H), 5.03-4.97/4.76-4.69 (m, 1H), 3.14-3.04/2.96-2.86 (m, 1H), 2.63-2.18 (m, 5H), 1.80/1.48 (br d, 1H), 1.27/1.17 (d, J=6.7 Hz, 3H).

### Example 125B 2,7-dimethyl-1H-indene

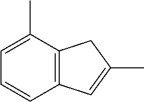

Using General procedure 7 and Example 125A as the appropriate indane, Example 125B was obtained as a colourless oil (2.37 g, 16.4 mmol, 90%). ¹H NMR (400 MHz, CDCl₃) δ ppm: 7.18-7.08 (m, 2H), 6.95-6.89 (m, 1H), 6.52-6.47 (m, 1H), 3.20 (s, 2H), 2.33 (s, 3H), 2.17 (s, 3H).

### Example 125C 2″,7″-dimethyldispiro[[1,3]dioxolane-2,1′-cyclohexane-4′,1″-indene]

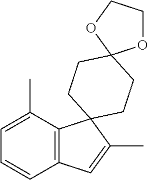 [SMILES: CC1=Cc2cccc(C)c2C12CCC1(CC2)OCCO1]

Using General procedure 8a with Preparation 1a and Example 125B as the appropriate indene, a mixture of regioisomers was obtained. They were separated by flash chromatography using heptane and EtOAc as eluents to obtain Example 125C as a yellow oil (238 mg, 0.88 mmol, 13%). ¹H NMR (400 MHz, CDCl₃) δ ppm: 7.11-7.04 (m, 1H), 7.01-6.96 (m, 1H), 6.91-6.86 (m, 1H), 6.28-6.23 (m, 1H), 4.08-3.99 (m, 4H), 2.79 (td, J=14.4, 5.0 Hz, 2H), 2.56 (s, 3H), 2.29 (d, J=1.5 Hz, 3H), 2.12 (td, J=14.0, 5.1 Hz, 2H), 1.85-1.76 (m, 2H), 1.50-1.41 (m, 2H).

### Example 125D 2′,7′-dimethylspiro[cyclohexane-1,1′-inden]-4-one

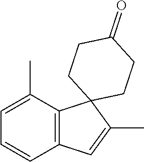 [SMILES: CC1=Cc2cccc(C)c2C12CCC(=O)CC2]

Using General procedure 9 and Example 125C as the appropriate ketal, Example 125D was obtained as a yellow solid (120 mg, 0.53 mmol, 81%). ¹H NMR (400 MHz, CDCl₃) δ ppm: 7.15-7.08 (m, 1H), 7.07-7.01 (m, 1H), 6.94-6.88 (m, 1H), 6.38-6.32 (m, 1H), 2.88-2.70 (m, 4H), 2.67-2.55 (m, 2H), 2.49 (s, 3H), 2.26 (d, J=1.4 Hz, 3H), 1.92-1.80 (m, 2H).

### Example 125E 4-(3-bromoanilino)-2′,7′-dimethylspiro[cyclohexane-1,1′-indene]-4-carboxamide

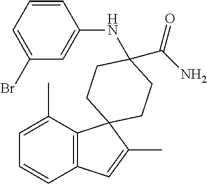 [SMILES: CC1=Cc2cccc(C)c2C12CCC(Nc1cccc(Br)c1)(C(N)=O)CC2]

Using General procedure 11 and Example 125D as the appropriate ketone and 3-bromoaniline as the appropriate aniline afforded an intermediate that was treated according to General Procedure 12b to obtain a mixture of diastereoisomers, Example 125E as a yellow solid (45 mg, 0.11 mmol, 20%). ¹H NMR (400 MHz, CDCl₃) δ ppm: 7.12-7.04 (m, 2H), 7.00 (d, J=7.3 Hz, 1H), 6.95 (d, J=8.0 Hz, 1H), 6.90-6.77 (m, 3H), 6.61 (d, J=8.2 Hz, 1H), 6.28 (s, 1H), 5.43 (br s, 1H), 4.29 (s, 1H), 2.71-2.59 (m, 2H), 2.57 (s, 3H), 2.51-2.40 (m, 2H), 2.37 (s, 3H), 2.20-2.11 (m, 2H), 1.44-1.34 (m, 2H).

### Example 125 4-(3-bromoanilino)-2′,7′-dimethylspiro[cyclohexane-1,1′-indene]-4-carboxylic acid

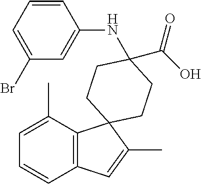 [SMILES: CC1=Cc2cccc(C)c2C12CCC(Nc1cccc(Br)c1)(C(=O)O)CC2]

Using General procedure 13 and Example 125E as the appropriate amide, Example 125 was obtained as a yellow solid (6 mg, 0.014 mmol, 41%) that was a mixture of diastereoisomers. LRMS calculated for C₂₃H₂₄BrNO₂: 425; found: 426 (M+H). ¹H NMR (400 MHz, DMSO-d₆) δ ppm: 12.71 (br s, 1H), 7.05 (t, J=8.0 Hz, 1H), 7.01 (t, J=7.4 Hz, 1H), 6.98-6.93 (m, 1H), 6.84-6.79 (m, 1H), 6.75 (t, J=2.0 Hz, 1H), 6.74-6.69 (m, 1H), 6.63-6.57 (m, 1H), 6.46 (br s, 1H), 6.30-6.26 (m, 1H), 2.71-2.58 (m, 2H), 2.48 (s, 3H), 2.33-2.16 (m, 7H), 1.21-1.12 (m, 2H).

### Example 126 and Example 127 Example 126A (2E)-2-benzylidene-6-methoxy-2,3-dihydro-1H-inden-1-one

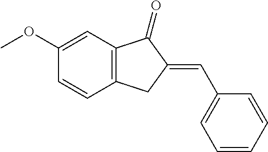 [SMILES: COc1ccc2c(c1)C(=O)/C(=C/c1ccccc1)C2]

To a solution of 6-methoxy-2,3-dihydro-1H-inden-1-one (1 g, 6.17 mmol, 1 eq) and benzaldehyde (823 μL, 6.47 mmol, 1.05 eq) in EtOH (6 mL) was added 6 M aq. NaOH solution (1.54 mL, 9.25 mmol, 1.5 eq) dropwise and the mixture was stirred at rt for 18 h. The resultant solid was collected by filtration and dried under vacuum to afford Example 126A as a single diastereoisomer, isolated as a white solid (1.48 g, 5.9 mmol, 96%). ¹H NMR (400 MHz, DMSO-d₆) δ ppm: 7.84-7.77 (m, 2H), 7.60 (d, J=8.3 Hz, 1H), 7.57-7.44 (m, 4H), 7.32 (dd, J=8.3, 2.6 Hz, 1H), 7.27 (d, J=2.5 Hz, 1H), 4.07 (d, J=2.1 Hz, 2H), 3.85 (s, 3H).

### Example 126B 2-benzyl-6-methoxy-2,3-dihydro-1H-inden-1-one

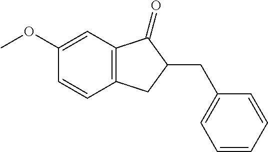 [SMILES: COc1ccc2c(c1)C(=O)C(Cc1ccccc1)C2]

To a solution of Example 126A (1.48 g, 5.92 mmol, 1 eq) in EtOAc (100 mL) was added 10% Pd/C (catalytic) under a N₂ atmosphere. The mixture was evacuated and backfilled with N₂ (×3), then evacuated and backfilled with H₂ and shaken for 5 days at rt under an atmosphere of H₂. The reaction was filtered through celite, eluted with EtOAc and the filtrate was concentrated under reduced pressure. Purification by automated flash column chromatography (CombiFlash Rf, 40 g RediSep™ silica cartridge) eluting with a gradient of 0-8% EtOAc in heptane afforded a racemate, Example 126B as a colourless oil (1.21 g, 4.78 mmol, 81%). ¹H NMR (400 MHz, DMSO-d₆) δ ppm: 7.43 (d, J=8.3 Hz, 1H), 7.32-7.23 (m, 5H), 7.23-7.18 (m, 1H), 7.13 (d, J=2.5 Hz, 1H), 3.81 (s, 3H), 3.23-3.13 (m, 1H), 3.12-3.00 (m, 2H), 2.79-2.64 (m, 2H).

### Example 126C (1E)-2-benzyl-6-methoxy-1-(methoxymethylidene)-2,3-dihydro-1H-indene and (1Z)-2-benzyl-6-methoxy-1-(methoxymethylidene)-2,3-dihydro-1H-indene

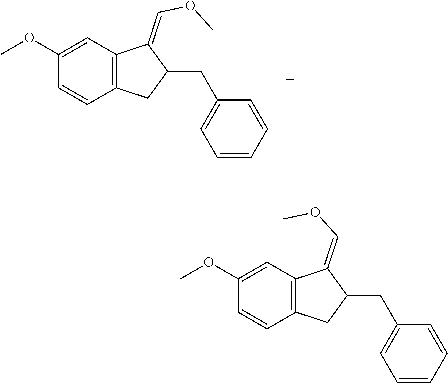 [SMILES: CO/C=C1/c2cc(OC)ccc2CC1Cc1ccccc1 ; CO/C=C1\c2cc(OC)ccc2CC1Cc1ccccc1]

To a suspension of KOtBu (1.23 g, 10.9 mmol, 2.3 eq) in 1,4-dioxane (25 mL) was added (methoxymethyl)triphenylphosphonium chloride (3.75 g, 10.9 mmol, 2.3 eq) and the mixture was stirred at rt for 2 h. Example 126B (1.2 g, 4.76 mmol, 1 eq) in 1,4-dioxane (25 mL) was added and the mixture was stirred at rt for 18 h, then the volatiles were evaporated under reduced pressure. Purification by automated flash column chromatography (CombiFlash Rf, 40 g RediSep™ silica cartridge) eluting with a gradient of 0-60% EtOAc in heptane afforded a racemic mixture of diastereoisomers, Example 126C (575 mg, 2.05 mmol, 43%). LRMS calculated for C₁₉H₂₀O₂: 280; found: 281 (M+H).

### Example 126D 2′-benzyl-6′-methoxy-2′,3′-dihydrospiro[cyclohex-2-ene-1,1′-inden]-4-one

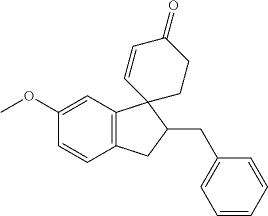 [SMILES: COc1ccc2c(c1)C1(C=CC(=O)CC1)C(Cc1ccccc1)C2]

To a solution of Example 126C (575 mg, 2.05 mmol, 1 eq) in toluene (10 mL) was added 3-buten-2-one (188 μL, 2.26 mmol, 1.1 eq) and PTSA (39 mg, 0.21 mmol, 0.1 eq) and the mixture was heated at 85° C. for 18 h. The reaction was allowed to cool to rt and partitioned between EtOAc and sat. aq. NaHCO₃ solution. The organic phase was washed with brine, dried (MgSO₄) and concentrated in vacuo. Purification by automated flash column chromatography (CombiFlash Rf, 40 g RediSep™ silica cartridge) eluting with a gradient of 0-30% EtOAc in heptane afforded a racemic mixture of diastereoisomers, Example 126D as a colourless oil (267 mg, 0.84 mmol, 41%). LRMS calculated for C₂₂H₂₂O₂: 318; found: 319 (M+H).

### Example 126E 2′-benzyl-6′-methoxy-2′,3′-dihydrospiro[cyclohexane-1,1′-inden]-4-one

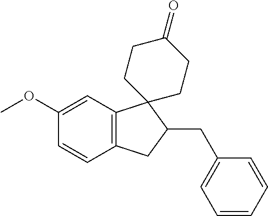 [SMILES: COc1ccc2c(c1)C1(CCC(=O)CC1)C(Cc1ccccc1)C2]

To a solution of Example 126D (267 mg, 0.84 mmol, 1 eq) in AcOH (50 mL) was added 10% Pd/C (catalytic) under a N₂ atmosphere. The mixture was evacuated and backfilled with N₂ (×3), then evacuated and backfilled with H₂ and shaken for 18 h at rt under an atmosphere of H₂. The reaction was filtered through celite, eluted with DCM, then the filtrate was concentrated under reduced pressure to afford a racemate, Example 126E as a colourless oil. LRMS calculated for C₂₂H₂₄O₂: 320; found: 321 (M+H).

### Example 126 (1s,4s)-2′-benzyl-4-(3-bromoanilino)-6′-methoxy-2′,3′-dihydrospiro[cyclohexane-1,1′-indene]-4-carboxylic acid

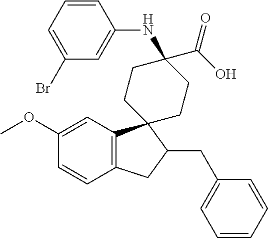

and

### Example 127 (1r,4r)-2′-benzyl-4-(3-bromoanilino)-6′-methoxy-2′,3′-dihydrospiro[cyclohexane-1,1′-indene]-4-carboxylic acid

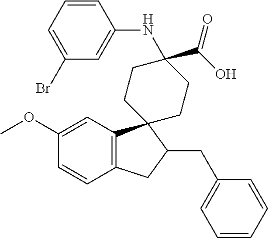

Using General Procedure 10 and Example 126E as the appropriate ketone and 3-bromoaniline as the appropriate aniline, a mixture of diastereoisomers was obtained. They were purified and separated by automated flash column chromatography (CombiFlash Rf, 40 g RediSep™ silica cartridge) eluting with a gradient of 0-20% EtOAc in heptane. The racemic diastereoisomer eluting earlier was collected as Example 126, isolated as a white solid (60.7 mg, 0.12 mmol, 14%). LRMS calculated for C₂₉H₃₀BrNO₃: 519; found: 520 (M+H). ¹H NMR (400 MHz, DMSO-d₆) δ ppm: 12.68 (br s, 1H), 7.35-7.28 (m, 2H), 7.25-7.15 (m, 3H), 7.07 (d, J=8.1 Hz, 1H), 7.03 (t, J=8.1 Hz, 1H), 6.83 (t, J=2.1 Hz, 1H), 6.75 (d, J=2.4 Hz, 1H), 6.74-6.68 (m, 2H), 6.64-6.59 (m, 1H), 6.26 (br s, 1H), 3.73 (s, 3H), 2.89-2.80 (m, 1H), 2.78-2.68 (m, 1H), 2.67-2.58 (m, 1H), 2.40-2.32 (m, 1H), 2.24-1.95 (m, 6H), 1.74-1.65 (m, 1H), 1.62-1.52 (m, 2H).

The racemic diastereoisomer eluting later was collected as Example 127, isolated as a white solid (8.5 mg, 0.02 mmol, 2%). LRMS calculated for C₂₉H₃₀BrNO₃: 519; found: 520 (M+H). ¹H NMR (400 MHz, DMSO-d₆) δ ppm: 7.34-7.28 (m, 2H), 7.24-7.17 (m, 3H), 7.06 (d, J=8.2 Hz, 1H), 7.02 (t, J=8.1 Hz, 1H), 6.90 (d, J=2.4 Hz, 1H), 6.82 (t, J=2.1 Hz, 1H), 6.74-6.68 (m, 2H), 6.63-6.58 (m, 1H), 3.73 (s, 3H), 2.93-2.83 (m, 1H), 2.63-2.54 (m, 1H), 2.50-2.38 (m, 2H), 2.36-2.16 (m, 3H), 2.06-1.81 (m, 4H), 1.59-1.48 (m, 2H).

### Example 128

### Example 128A 6-methoxy-2,2-dimethyl-2,3-dihydro-1H-inden-1-one

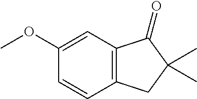

To a solution of 6-methoxy-2,3-dihydro-1H-inden-1-one (0.81 g, 5 mmol, 1 eq) in THE (25 mL) was added NaH (60% dispersion; 0.44 g, 11 mmol, 2.2 eq) in portions and the mixture was stirred for 45 min before the dropwise addition of MeI (0.68 mL, 11 mmol, 2.2 eq). The mixture was stirred at rt for 18 h, then quenched with sat. aq. NH₄Cl solution. The mixture was partitioned between EtOAc and dilute aq. HCl solution, then the combined organic extracts were washed with brine, dried (MgSO₄) and concentrated in vacuo. Purification by automated flash chromatography (CombiFlash Rf, 40 g RediSep™ silica cartridge) eluting with a gradient of 0-11% EtOAc in heptane afforded Example 128A as a yellow oil (0.84 g, 4.4 mmol, 88%). ¹H NMR (400 MHz, DMSO-d₆) δ ppm: 7.46 (dq, J=8.4, 0.7 Hz, 1H), 7.29 (dd, J=8.3, 2.6 Hz, 1H), 7.12 (d, J=2.5 Hz, 1H), 3.81 (s, 3H), 2.92 (d, J=0.9 Hz, 2H), 1.14 (s, 6H).

### Example 128B 6-methoxy-1-(methoxymethylidene)-2,2-dimethyl-2,3-dihydro-1H-indene

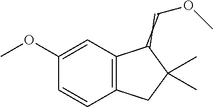

To a suspension of (methoxymethyl)triphenylphosphonium chloride (3.6 g, 28.8 mmol, 2.4 eq) in MeOH (30 mL), cooled to −5° C.-0° C., was added KOtBu (0.98 g, 8.7 mmol, 2 eq) and the mixture was stirred for 2 h. Example 128A (0.83 g, 4.37 mmol, 1 eq) in MeOH (5 mL) was added dropwise at −5° C.-0° C. and then stirred for a further 30 min, before warming to rt and stirring for 72 h. The mixture was partitioned between EtOAc and water, and the organic phase was washed with brine, dried (MgSO₄) and concentrated in vacuo. EtOAc/hexane (1:1) was added and the formed precipitate (PPh₃0) was filtered, washed with EtOAc/hexane (1:1). The filtrate was concentrated in vacuo. Purification by automated flash chromatography (CombiFlash Rf, 80 g RediSep™ silica cartridge) eluting with a gradient of 0-10% EtOAc in heptane afforded a 3:1 mixture of E and Z isomers, Example 128B as an oil (0.42 g, 1.93 mmol, 44%). ¹H NMR (400 MHz, DMSO-d₆) δ ppm: 7.23/6.88 (d, J=2.5 Hz, 1H), 7.09-6.98 (m, 1H), 6.86/6.24 (s, 1H), 6.69/6.62 (dd, J=8.2, 2.5 Hz, 1H), 3.72 (s, 3H), 3.71/3.67 (s, 3H), 2.69-2.66 (m, 2H), 1.27/1.17 (s, 6H).

### Example 128C 6′-methoxy-2′,2′-dimethyl-2′,3′-dihydrospiro[cyclohex-2-ene-1,1′-inden]-4-one

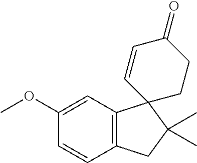

To a solution of Example 128B (422 mg, 1.93 mmol) in toluene (8 mL) was added MVK (298 mg, 354 uL, 4.24 mmol) and PTSA (33 mg, 0.19 mmol, 0.1 eq). The mixture was heated at 100° C. for 36 h. The mixture was allowed to cool tort and poured into sat. aq. NaHCO₃ solution. The reaction was partitioned between EtOAc and water, and the organic phase was washed with brine, dried (MgSO₄) and concentrated in vacuo. Purification by automated flash chromatography (CombiFlash Rf, 40 g RediSep™ silica cartridge) eluting with a gradient of 0-30% EtOAc in heptane afforded a racemate, Example 128C as a yellow oil (163 mg, 0.64 mmol, 33%). LRMS calculated for C₁₇H₂₀O₂: 256; found: 257 (M+H).

### Example 128D 6′-methoxy-2′,2′-dimethyl-2′,3′-dihydrospiro[cyclohexane-1,1′-inden]-4-one

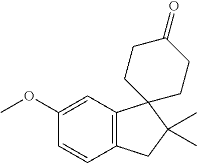

To a solution of Example 128C (150 mg, 0.59 mmol, 1 eq) in AcOH (5 mL) was added 10% Pd/C (25 mg) under a N₂ atmosphere. The mixture was evacuated and backfilled with N₂ (×3), then evacuated and backfilled with H₂ and shaken for 3 h at rt under an atmosphere of H₂. The reaction was filtered through celite, eluted with DCM and the volatiles were evaporated under reduced pressure to afford Example 128D as a yellow oil (140 mg, 0.54 mmol, 93%). LRMS calculated for C₁₇H₂₂O₂: 258; found: 259 (M+H).

### Example 128 (1s,4s)-4-(3-bromoanilino)-6′-methoxy-2′,2′-dimethyl-2′,3′-dihydrospiro[cyclohexane-1,1′-indene]-4-carboxylic acid

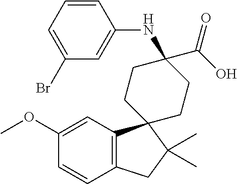 [SMILES: COc1ccc2c(c1)[C@]1(CC[C@](Nc3cccc(Br)c3)(C(=O)O)CC1)C(C)(C)C2]

Using General procedure 10 and Example 128D as the appropriate ketone and 3-bromoaniline as the appropriate aniline, a mixture of diastereoisomers was obtained.

Purification by automated flash chromatography (CombiFlash Rf, 24 g RediSep™ silica cartridge) eluting with a gradient of 0-20% EtOAc in heptane afforded the major single diastereoisomer, Example 128 as a white solid (12 mg, 0.03 mmol, 5%). LRMS calculated for C₂₄H₂₈BrNO₃: 457; found: 458 (M+H). ¹H NMR (400 MHz, DMSO-d₆) δ ppm: 12.73 (br s, 1H), 7.07 (d, J=8.1 Hz, 1H), 7.01 (t, J=8.1 Hz, 1H), 6.90 (d, J=2.4 Hz, 1H), 6.81 (t, J=2.1 Hz, 1H), 6.74-6.67 (m, 2H), 6.61 (dd, J=8.1, 2.2 Hz, 1H), 6.30 (br s, 1H), 3.72 (s, 3H), 2.60 (s, 2H), 2.33-2.25 (m, 2H), 1.86 (t, J=10.6 Hz, 2H), 1.72 (t, J=12.7 Hz, 2H), 1.62-1.53 (m, 2H), 0.96 (s, 6H).

### Example 129 (1s,4s)-2′-bromo-4-(3-chloroanilino)-6′-methoxyspiro[cyclohexane-1,1′-indene]-4-carboxylic acid

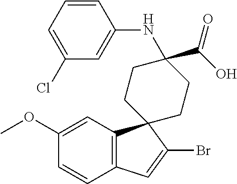 [SMILES: COc1ccc2c(c1)[C@]1(CC[C@@](Nc3cccc(Cl)c3)(C(=O)O)CC1)C(Br)=C2]

Preparation 18aH was purified via prep RP-HPLC using 25 mM aq. NH₄HCO₃ solution and MeCN as eluents to give Example 129. ¹H NMR (500 MHz, DMSO-d₆) δ ppm: 7.34 (d, 1H), 7.26 (d, 1H), 7.03 (t, 1H), 6.92 (s, 1H), 6.88 (dd, 1H), 6.60 (t, 1H), 6.54 (dm, 1H), 6.50 (dm, 1H), 6.26 (br s, 1H), 3.77 (s, 3H), 2.40/2.17 (m+m, 4H), 2.14/0.95 (m+m, 4H). HRMS calculated for C₂₂H₂₁NO₃ClBr: 461.0393; found: 464.0451 (M+H).

### Example 130

### Example 130A 2′-bromo-5′-methoxy-spiro[cyclohexane-4,1′-indene]-1-one

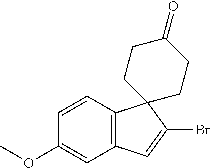 [SMILES: COc1ccc2c(c1)C=C(Br)C21CCC(=O)CC1]

Using General procedure 9 and Preparation 18aD as the appropriate ketal, Example 130A was obtained. ¹H NMR (500 MHz, DMSO-d₆) δ ppm: 7.82 (d, 1H), 7.06 (s, 1H), 6.99 (d, 1H), 6.75 (dd, 1H), 3.77 (s, 3H), 2.92/2.46 (dd+dd, 4H), 2.20/1.52 (td+m, 4H). HRMS calculated for C₁₅H₁₅BrO₂: 306.0255; found 306.0239 (M+H).

### Example 130B (1s,4s)-2′-bromo-4-(3-chloroanilino)-5′-methoxyspiro[cyclohexane-1,1′-indene]-4-carbonitrile

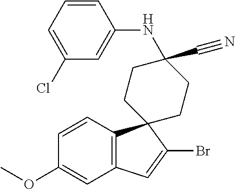 [SMILES: COc1ccc2c(c1)C=C(Br)[C@]21CC[C@@](C#N)(Nc2cccc(Cl)c2)CC1]

Using General procedure 11 and Example 130A as the appropriate ketone and 3-chloroaniline as the appropriate aniline, a mixture of diastereoisomers was obtained. The diastereoisomers were separated via flash chromatography using heptane and EtOAc as eluents. The diastereoisomer eluting later was collected as Example 130B. ¹H NMR (500 MHz, DMSO-d₆) δ ppm: 7.67 (d, 1H), 7.24 (t, 1H), 7.01 (s, 1H), 6.96 (d, 1H), 6.93 (t, 1H), 6.89 (dm, 1H), 6.79 (dm, 1H), 6.79 (dd, 1H), 6.58 (s, 1H), 3.76 (s, 3H), 2.56/2.45 (d+td, 4H), 2.09/1.20 (td+d, 4H). HRMS calculated for C₂₂H₂₀BrClN₂O: 442.0447; found 443.0524 (M+H).

### Example 130C (1s,4s)-2′-bromo-4-(3-chloroanilino)-5′-methoxyspiro[cyclohexane-1,1′-indene]-4-carboxamide

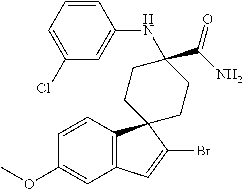 [SMILES: COc1ccc2c(c1)C=C(Br)[C@]21CC[C@](Nc2cccc(Cl)c2)(C(N)=O)CC1]

Using General procedure 12a and Example 130B as the appropriate nitrile, Example 130C was obtained. ¹H NMR (500 MHz, DMSO-d₆) δ ppm: 7.64 (d, 1H), 7.33/7.24 (s+s, 2H), 7.11 (t, 1H), 6.97 (s, 1H), 6.95 (d, 1H), 6.79 (dd, 1H), 6.69 (dd, 1H), 6.62 (dd, 1H), 6.60 (dd, 1H), 6.21 (s, 1H), 3.75 (s, 3H), 2.43/2.09 (t+d, 4H), 2.10/0.90 (t+d, 4H). HRMS calculated for C₂₂H₂₂BrClN₂O₂: 460.0553; found 461.0623 (M+H).

### Example 130 (1s,4s)-2′-bromo-4-(3-chloroanilino)-5′-methoxyspiro[cyclohexane-1,1′-indene]-4-carboxylic acid

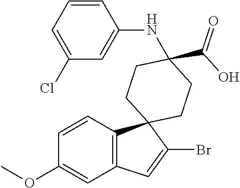 [SMILES: COc1ccc2c(c1)C=C(Br)[C@]21CC[C@](Nc2cccc(Cl)c2)(C(=O)O)CC1]

Using General procedure 13 and Example 130C as the appropriate amide, Example 130 was obtained. ¹H NMR (500 MHz, DMSO-d₆) δ ppm: 7.65 (d, 1H), 7.00 (t, 1H), 6.95 (s, 1H), 6.94 (d, 1H), 6.77 (dd, 1H), 6.61 (t, 1H), 6.55 (dm, 1H), 6.45 (dm, 1H), 6.15 (br, 1H), 3.75 (s, 3H), 2.36/2.14 (td+d, 4H), 2.12/0.88 (t+d, 4H). HRMS calculated for C₂₂H₂₁NO₃ClBr: 461.0393; found: 462.0465 (M+H).

### Example 132 (1s,4s)-6′-acetyl-2′-bromo-4-(3-chloroanilino)spiro[cyclohexane-1,1′-indene]-4-carboxylic acid

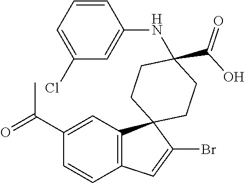 [SMILES: CC(=O)c1ccc2c(c1)[C@]1(CC[C@@](Nc3cccc(Cl)c3)(C(=O)O)CC1)C(Br)=C2]

Using General procedure 33a and Preparation 17aA as the appropriate ester, Example 132 was obtained. ¹H NMR (500 MHz, DMSO-d₆) δ ppm: 9.43 (s, 1H), 8.32 (s, 1H), 7.97 (dd, 1H), 7.50 (d, 1H), 7.16 (s, 1H), 7.06 (t, 1H), 6.63 (t, 1H), 6.55 (dd, 1H), 6.52 (dd, 1H), 6.40 (s, 1H), 2.59 (s, 3H), 2.43/2.26 (m, 4H), 2.2/1.00 (m, 4H). HRMS calculated for C₂₃H₂₁NO₃ClBr: 473.0393; found: 474.0468 (M+H).

### Example 133 (1s,4s)-2′-bromo-4-(3-chloroanilino)-6′-(1-hydroxyethyl)spiro[cyclohexane-1,1′-indene]-4-carboxylic acid

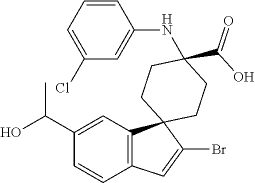 [SMILES: CC(O)c1ccc2c(c1)[C@]1(CC[C@@](Nc3cccc(Cl)c3)(C(=O)O)CC1)C(Br)=C2]

Using General procedure 36 and Preparation 17aA as the appropriate acetyl derivative, then hydrolyzing the obtained ester according to General procedure 33a resulted in Example 133 as a racemate. ¹H NMR (500 MHz, DMSO-d₆) δ ppm: 7.75 (d, 1H), 7.28 (d, 1H), 7.26 (dd, 1H), 7.06 (t, 1H), 6.99 (s, 1H), 6.63 (t, 1H), 6.57 (dd, 1H), 6.54 (dd, 1H), 6.35 (s, 1H), 4.72 (q, 1H), 2.43-0.96 (m, 8H), 1.35 (d, 3H). HRMS calculated for C₂₃H₂₃NO₃ClBr: 475.0550; found: 476.0619 (M+H).

### Example 134

### Example 134A 2-bromo-5-chloro-2,3-dihydro-1H-inden-1-one

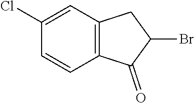 [SMILES: O=C1c2ccc(Cl)cc2CC1Br]

Using General procedure 5 and 5-chloroindan-1-one as the appropriate indan-1-one, Example 134A was obtained. ¹H NMR (500 MHz, DMSO-d₆) δ ppm: 7.76 (d, 1H), 7.71 (d, 1H), 7.56 (dd, 1H), 5.03 (dd, 1H), 3.88+3.33 (dd+dd, 2H).

### Example 134B rac-(1R,2S)-2-bromo-5-chloro-2,3-dihydro-1H-inden-1-ol

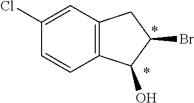 [SMILES: O[C@H]1c2ccc(Cl)cc2C[C@H]1Br]

Using General procedure 6 and Example 134A as the appropriate bromo-indan-1-one, Example 134B was obtained. ¹H NMR (500 MHz, DMSO-d₆) δ ppm: 7.40-7.22 (m, 3H), 5.90 (d, 1H), 4.93 (m, 1H), 4.89 (m, 1H), 3.44+3.19 (dd+dd, 2H).

### Example 134C 2-bromo-6-chloro-1H-indene

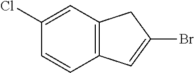 [SMILES: Clc1ccc2c(c1)CC(Br)=C2]

Using General procedure 7 and Example 134B as the appropriate indane, Example 134C was obtained. ¹H NMR (500 MHz, DMSO-d₆) δ ppm: 7.48 (br s, 1H), 7.37 (d, 1H), 7.31 (dd, 1H), 7.13 (s, 1H), 3.74 (s, 2H).

### Example 134D 2′-bromo-6′-chlorospiro[cyclohexane-1,1′-inden]-4-one

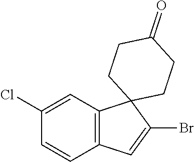 [SMILES: O=C1CCC2(CC1)C(Br)=Cc1ccc(Cl)cc12]

and

### Example 134E 2′-bromo-5′-chlorospiro[cyclohexane-1,1′-inden]-4-one

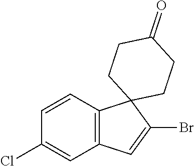 [SMILES: O=C1CCC2(CC1)C(Br)=Cc1cc(Cl)ccc12]

Using General procedure 8a and Example 134C as the appropriate indene, a mixture of regioisomers was obtained. Then the ketal was hydrolyzed according to General procedure 9. The regioisomers were separated via flash chromatography using hexane and acetone as eluents. The regioisomer eluting earlier was collected as Example 134D. ¹H NMR (500 MHz, DMSO-d₆) δ ppm: 8.00 (br s, 1H), 7.40 (d, 1H), 7.38 (dd, 1H), 7.11 (s, 1H), 2.89 (ddd, 2H), 2.51 (m, 2H), 2.18 (td, 2H), 1.67 (dm, 2H).

The regioisomer eluting later was collected as Example 134E. ¹H NMR (500 MHz, DMSO-d₆) δ ppm: 7.93 (d, 1H), 7.48 (d, 1H), 7.25 (dd, 1H), 7.10 (s, 1H), 2.88 (ddd, 2H), 2.50 (m, 2H), 2.21 (td, 2H), 1.59 (m, 2H).

### Example 134F 2″-bromo-6″-chlorodispiro[imidazolidine-4,1′-cyclohexane-4′,1″-indene]-2,5-dione

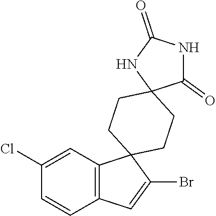 [SMILES: O=C1NC(=O)C2(CCC3(CC2)C(Br)=Cc2ccc(Cl)cc23)N1]

Using General procedure 14 and Example 134D as the appropriate ketone, Example 134F was obtained as a mixture of diastereoisomers. ¹H NMR (500 MHz, DMSO-d₆) δ ppm: 8.71/8.56 (br, 1H), 7.98/7.69 (s/s, 1H), 7.39/7.32 (s/s, 2H), 7.07/7.06 (s/s, 1H), 2.43/2.29 (br/td, 2H), 2.10/1.96 (td/br, 2H), 1.82/1.76 (m/d, 2H), 1.76/1.21 (m/d, 2H).

### Example 134G 4-amino-2′-bromo-6′-chlorospiro[cyclohexane-1,1′-indene]-4-carboxylic acid

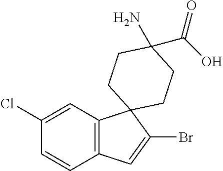 [SMILES: NC1(C(=O)O)CCC2(CC1)C(Br)=Cc1ccc(Cl)cc12]

Using General procedure 15 and Example 134F as the appropriate hydantoin, Example 134G was obtained as a mixture of diastereoisomers. ¹H NMR (500 MHz, DMSO-d₆) δ ppm: 8.08/8.05 (s/s, 1H), 8.00-6.80 (br, 2H), 7.36/7.33 (s/s, 1H), 7.04/7.01 (s/s, 1H), 2.55/2.40 (t/t, 2H), 2.29/1.80 (br d/d, 2H), 2.09/1.92 (t/t, 2H), 1.16/1.10 (d/d, 2H).

### Example 134 (1s,4s)-2′-bromo-6′-chloro-4-(3-chloroanilino)spiro[cyclohexane-1,1′-indene]-4-carboxylic acid

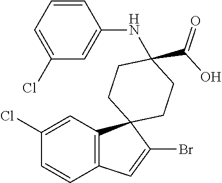 [SMILES: O=C(O)[C@]1(Nc2cccc(Cl)c2)CC[C@]2(CC1)C(Br)=Cc1ccc(Cl)cc12]

Using General procedure 16 and Example 134G as the appropriate amino acid and 1-chloro-3-iodo-benzene as the appropriate iodobenzene, a mixture of diastereoisomers was obtained. The diastereoisomers were separated by chiral chromatography. Column: AD, 50×500 mm, 20 μm, Eluents: iPrOH/heptane+HCOOH. The diastereoisomer eluting later was collected as Example 134. ¹H NMR (500 MHz, DMSO-d₆) δ ppm: 7.67 (s, 1H), 7.39 (s, 1H), 7.39 (s, 1H), 7.09 (t, 1H), 7.06 (s, 1H), 6.61 (t, 1H), 6.57 (dd, 1H), 6.55 (dd, 1H), 2.32/2.26 (td+d, 4H), 2.19/1.02 (td+d, 4H). HRMS calculated for C₂₁H₁₈NO₂Cl₂Br: 464.9898; found: 465.9970 (M+H).

### Example 135

### Example 135A 2″-bromo-5″-chlorodispiro[imidazolidine-4,1′-cyclohexane-4′,1″-indene]-2,5-dione

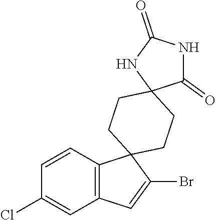 [SMILES: O=C1NC(=O)C2(CCC3(CC2)C(Br)=Cc2cc(Cl)ccc23)N1]

Using General procedure 14 and Example 134E as the appropriate ketone, Example 135A was obtained as a mixture of diastereoisomers. ¹H NMR (500 MHz, DMSO-d₆) δ ppm: 10.78/10.62 (br s/br s, 1H), 8.93/8.40 (s/s, 1H), 7.87/7.67 (d/d, 1H), 7.45/7.40 (d/d, 1H), 7.33/7.26 (dd/dd, 1H), 7.05/7.04 (s/s, 1H), 2.31 (td, 2H), 2.11 (td, 2H), 1.77 (d, 2H), 1.18 (d, 2H).

### Example 135B 4-amino-2′-bromo-5′-chlorospiro[cyclohexane-1,1′-indene]-4-carboxylic acid

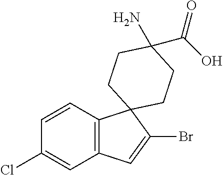 [SMILES: NC1(C(=O)O)CCC2(CC1)C(Br)=Cc1cc(Cl)ccc12]

Using General procedure 15 and Example 135A as the appropriate hydantoin, Example 135B was obtained as a mixture of diastereoisomers. ¹H NMR (500 MHz, DMSO-d₆) δ ppm: 8.05/8.01 (d, 1H), 7.43/7.40 (d, 1H), 7.25/7.20 (dd, 1H), 7.03/6.99 (s, 1H), 2.67-0.99 (m, 8H). HRMS calculated for C₁₅H₁₅BrClNO₂: 354.9975; found 356.0041 and 356.0039 (M+H).

### Example 135 (1s,4s)-2′-bromo-5′-chloro-4-(3-chloroanilino)spiro[cyclohexane-1,1′-indene]-4-carboxylic acid

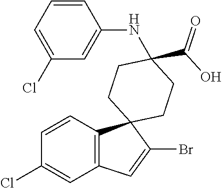 [SMILES: O=C(O)[C@]1(Nc2cccc(Cl)c2)CC[C@]2(CC1)C(Br)=Cc1cc(Cl)ccc12]

Using General procedure 16 and Example 135B as the appropriate amino acid and 1-chloro-3-iodo-benzene as the appropriate iodobenzene, a mixture of diastereoisomers was obtained. The diastereoisomers were separated by chiral chromatography. Column: AD, 50×500 mm, 20 μm, Eluents: 50:50 iPrOH/heptane+0.05% HCOOH. The diastereoisomer eluting earlier was collected and further purified via prep RP-HPLC using 25 mM aq. NH₄HCO₃ solution and MeCN as eluents to give Example 135. ¹H NMR (500 MHz, DMSO-d₆) δ ppm: 7.69 (d, 1H), 7.45 (d, 1H), 7.31 (dd, 1H), 7.09 (t, 1H), 7.04 (s, 1H), 6.62 (t, 1H), 6.56 (m, 1H), 6.56 (m, 1H), 2.37-0.95 (m+m, 8H). HRMS calculated for C₂₁H₁₈BrCl₂NO₂: 464.9898; found 465.9968 (M+H).

### Example 136

### Example 136A 2-bromo-5,6-dimethoxy-2,3-dihydro-1H-inden-1-one

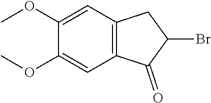

Using General procedure 5 and 5,6-dimethoxyindan-1-one as the appropriate indan-1-one Example 136A was obtained. ¹H NMR (500 MHz, DMSO-d₆) δ ppm: 7.15 (s, 1H), 7.13 (s, 1H), 4.95 (dd, 1H), 3.89 (s, 3H), 3.82 (s, 3H), 3.79 (dd, 1H), 3.21 (dd, 1H).

### Example 136B rac-(1R,2S)-2-bromo-5,6-dimethoxy-2,3-dihydro-1H-inden-1-ol

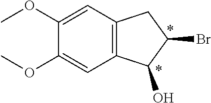

Using General procedure 6 and Example 136A as the appropriate bromo-indan-1-one, Example 136B was obtained as a racemic single diastereoisomer. ¹H NMR (500 MHz, CDCl₃) δ ppm: 6.96 (s, 1H), 6.75 (s, 1H), 4.94-4.83 (m, 2H), 3.87 (s, 3H), 3.86 (s, 3H), 3.35+3.28 (dd+dd, 2H).

### Example 136C 2-bromo-5,6-dimethoxy-1H-indene

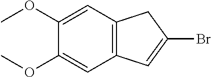

Using General procedure 7 and Example 136B as the appropriate indane, Example 136C was obtained. ¹H NMR (500 MHz, DMSO-d₆) δ ppm: 7.15-6.86 (m, 3H), 3.75 (s, 3H), 3.75 (s, 3H), 3.58 (s, 2H).

### Example 136D 2″-bromo-5″,6″-dimethoxydispiro[[1,3]dioxolane-2,1′-cyclohexane-4′,1″-indene]

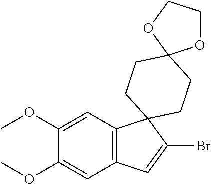 [SMILES: COc1cc2c(cc1OC)C1(CCC3(CC1)OCCO3)C(Br)=C2]

Using General procedure 8a and Example 136C as the appropriate indene, Example 136D was obtained. ¹H NMR (500 MHz, DMSO-d₆) δ ppm: 7.17 (s, 1H), 7.02 (s, 1H), 6.91 (s, 1H), 3.95 (m, 4H), 3.79 (s, 3H), 3.76 (s, 3H), 2.15-1.13 (m, 8H).

### Example 136E 2′-bromo-5′,6′-dimethoxyspiro[cyclohexane-1,1′-inden]-4-one

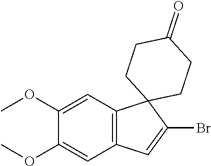 [SMILES: COc1cc2c(cc1OC)C1(CCC(=O)CC1)C(Br)=C2]

Using General procedure 9 and Example 136D as the appropriate ketal, Example 136E was obtained. ¹H NMR (500 MHz, DMSO-d₆) δ ppm: 7.43 (s, 1H), 7.04 (s, 1H), 6.97 (s, 1H), 3.81 (s, 3H), 3.77 (s, 3H), 2.94 (ddd, 2H), 2.52 (m, 2H), 2.16 (td, 2H), 1.64 (m, 2H). MS (EI, 70 eV) m/z (% relative intensity, [ion]): 115 (21), 144 (18), 201 (19), 215 (100), 257 (53), 336 (82, [M+]), 338 (80, [M+]).

### Example 136F (1′s,1″s)-2″-bromo-5″,6″-dimethoxydispiro[imidazolidine-4,1′-cyclohexane-4′,1″-indene]-2,5-dione

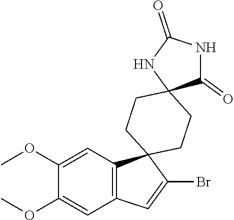 [SMILES: COc1cc2c(cc1OC)[C@]1(CC[C@@]3(CC1)NC(=O)NC3=O)C(Br)=C2]

Using General procedure 14 and Example 136E as the appropriate ketone, a mixture of diastereoisomers was obtained. The diastereoisomer mixture was recrystallized from CHCl₃ to give Example 136F. ¹H NMR (500 MHz, DMSO-d₆) δ ppm: 10.76 (br, 1H), 8.93 (s, 1H), 7.40 (s, 1H), 7.04 (s, 1H), 6.93 (s, 1H), 3.81 (s, 3H), 3.77 (s, 3H), 2.43 (td, 2H), 2.10 (td, 2H), 1.75 (d, 2H), 1.15 (d, 2H).

### Example 136G (1s,4s)-4-amino-2′-bromo-5′,6′-dimethoxyspiro[cyclohexane-1,1′-indene]-4-carboxylic acid

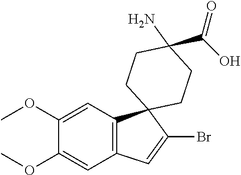 [SMILES: COc1cc2c(cc1OC)[C@]1(CC[C@@](N)(C(=O)O)CC1)C(Br)=C2]

Using General procedure 15 and Example 136F as the appropriate hydantoin, Example 136G was obtained. ¹H NMR (500 MHz, DMSO-d₆) δ ppm: 7.93 (s, 1H), 7.77 (br, 2H), 7.00 (s, 1H), 6.90 (s, 1H), 3.81 (s, 3H), 3.76 (s, 3H), 2.73 (td, 2H), 2.03 (td, 2H), 1.77 (d, 2H), 1.02 (d, 2H).

### Example 136 (1s,4s)-2′-bromo-4-(3-chloroanilino)-5′,6′-dimethoxyspiro[cyclohexane-1,1′-indene]-4-carboxylic acid

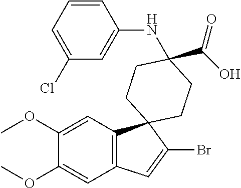 [SMILES: COc1cc2c(cc1OC)[C@]1(CC[C@@](Nc3cccc(Cl)c3)(C(=O)O)CC1)C(Br)=C2]

Using General procedure 16 and Example 136G as the appropriate amino acid and 1-chloro-3-iodo-benzene as the appropriate iodobenzene, Example 136 was obtained. ¹H NMR (500 MHz, DMSO-d₆) δ ppm: 7.38 (s, 1H), 7.07 (t, 1H), 7.03 (s, 1H), 6.91 (s, 1H), 6.59 (t, 1H), 6.55 (dt, 1H), 6.54 (dt, 1H), 6.38 (s, 1H), 3.77 (d, 6H), 2.46/2.21 (m, 4H), 2.14/0.95 (m, 4H). HRMS calculated for C₂₃H₂₃NO₄ClBr: 491.0499; found: 492.0571 (M+H).

### Example 137

### Example 137A 2-bromo-4-chloro-2,3-dihydro-1H-inden-1-one

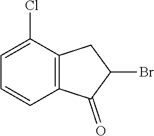 [SMILES: O=C1c2cccc(Cl)c2CC1Br]

Using General procedure 5 and 4-chloroindan-1-one as the appropriate indan-1-one, Example 137A was obtained. MS (EI, 70 eV) m/z (% relative intensity, [ion]): 75 (48), 102 (56), 137 (33), 165 (100), 167 (33), 244 (25, [M+]), 246 (31, [M+]), 248 (7, [M+]).

### Example 137B rac-(1R,2S)-2-bromo-4-chloro-2,3-dihydro-1H-inden-1-ol

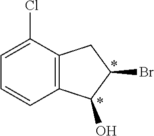 [SMILES: O[C@H]1c2cccc(Cl)c2C[C@H]1Br]

Using General procedure 6 and Example 137A as the appropriate bromo-indan-1-one, Example 137B was obtained as a racemic single diastereoisomer. MS (EI, 70 eV) m/z (% relative intensity, [ion]): 77 (38), 103 (72), 115 (49), 131 (45), 132 (45), 167 (100), 169 (32), 246 (21, [M+]), 248 (32, [M+]), 250 (8, [M+]).

### Example 137C 2-bromo-7-chloro-1H-indene

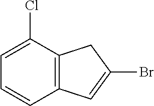 [SMILES: Clc1cccc2c1CC(Br)=C2]

Using General procedure 7 and Example 137B as the appropriate indane, Example 137C was obtained. MS (EI, 70 eV) m/z (% relative intensity, [ion]): 74 (21), 114 (26), 149 (100), 150 (31), 228 (17, [M+]), 230 (29, [M+]), 232 (5, [M+]).

### Example 137D 2″-bromo-4″-chlorodispiro[[1,3]dioxolane-2,1′-cyclohexane-4′,1″-indene] and 2″-bromo-7″-chlorodispiro[[1,3]dioxolane-2,1′-cyclohexane-4′,1″-indene]

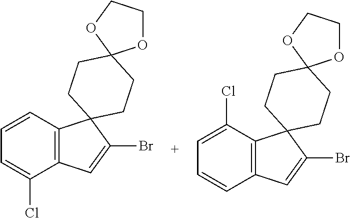 [SMILES: Clc1cccc2c1C=C(Br)C21CCC2(CC1)OCCO2 ; Clc1cccc2c1C1(CCC3(CC1)OCCO3)C(Br)=C2]

Using General procedure 8a and Example 137C as the appropriate indene, a mixture of regioisomers was obtained. ¹H NMR (500 MHz, DMSO-d₆) δ ppm: 7.69-7.15 (m, 3H), 7.08/7.04 (s/s, 1H), 3.99-3.89 (m, 4H), 3.06/2.53 (td/td, 2H), 2.12/2.07 (td/td, 2H), 1.88/1.71 (dm/dm, 2H), 1.40/1.22 (dm/dm, 2H).

### Example 137E 2′-bromo-4′-chlorospiro[cyclohexane-1,1′-inden]-4-one

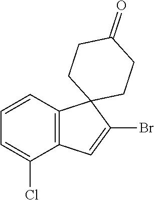 [SMILES: O=C1CCC2(CC1)C(Br)=Cc1c(Cl)cccc12]

Using General procedure 9 and Example 137D as the appropriate ketal, a mixture of regioisomers was obtained. The regioisomers were separated via flash chromatography using hexane and acetone as eluents. The regioisomer eluting later was collected as Example 137E. ¹H NMR (500 MHz, DMSO-d₆) δ ppm: 7.90 (d, 1H), 7.38 (d, 1H), 7.26 (t, 1H), 7.14 (s, 1H), 2.89 (ddd, 2H), 2.51 (dm, 2H), 2.22 (td, 2H), 1.64 (dm, 2H).

### Example 137F 2″-bromo-4″-chlorodispiro[imidazolidine-4,1′-cyclohexane-4′,1″-indene]-2,5-dione

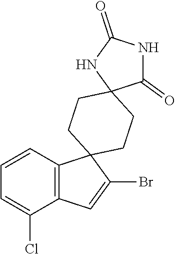 [SMILES: O=C1NC(=O)C2(CCC3(CC2)C(Br)=Cc2c(Cl)cccc23)N1]

Using General procedure 14 and Example 137E as the appropriate ketone, Example 137F was obtained as a mixture of diastereoisomers. LRMS calculated for C₁₆H₁₄BrClN₂O₂: 380.0; found 381.0 (M+H).

### Example 137G 4-amino-2′-bromo-4′-chlorospiro[cyclohexane-1,1′-indene]-4-carboxylic acid

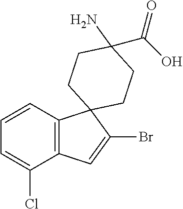 [SMILES: NC1(C(=O)O)CCC2(CC1)C(Br)=Cc1c(Cl)cccc12]

Using General procedure 15 and Example 137F as the appropriate hydantoin, Example 137G was obtained as a mixture of diastereoisomers. ¹H NMR (500 MHz, DMSO-d₆) δ ppm: 8.01/7.97 (d, 1H), 7.36/7.33 (d, 1H), 7.26/7.21 (t, 1H), 7.08/7.05 (s, 1H), 2.60/2.47 (td, 2H), 2.07/1.95 (td, 2H), 1.80/2.22 (d, 2H), 1.13 (d, 2H).

### Example 137 (1s,4s)-2′-bromo-4′-chloro-4-(3-chloroanilino)spiro[cyclohexane-1,1′-indene]-4-carboxylic acid

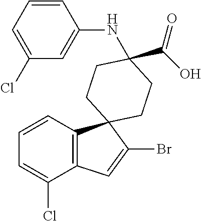 [SMILES: O=C(O)[C@]1(Nc2cccc(Cl)c2)CC[C@]2(CC1)C(Br)=Cc1c(Cl)cccc12]

Using General procedure 16 and Example 137G as the appropriate amino acid and 1-chloro-3-iodo-benzene as the appropriate iodobenzene, a mixture of diastereoisomers was obtained. The diastereoisomers were separated via chiral chromatography. Column: OJ, 100×500 mm, 20 μm, Eluents: EtOH/heptane+HCOOH. The diastereoisomer eluting earlier was collected as Example 137. ¹H NMR (500 MHz, DMSO-d₆) δ ppm: 12.89 (br s, 1H), 7.67 (d, 1H), 7.38 (d, 1H), 7.29 (t, 1H), 7.09 (s, 1H), 7.09 (t, 1H), 6.62 (t, 1H), 6.58 (dm, 1H), 6.56 (dm, 1H), 6.42 (br s, 1H), 2.35/2.26 (m+m, 4H), 2.21/1.04 (m+m, 4H). HRMS calculated for C₂₁H₁₈BrCl₂NO₂: 464.9898; found 465.9969 (M+H).

### Example 138

### Example 138A 2-bromo-5-fluoro-2,3-dihydro-1H-inden-1-one

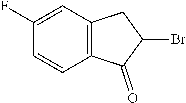 [SMILES: O=C1c2ccc(F)cc2CC1Br]

Using General procedure 5 and 5-fluoro-2,3-dihydro-1H-inden-1-one as the appropriate indan-1-one, Example 138A was obtained. ¹H NMR (500 MHz, DMSO-d₆) δ ppm: 7.83 (dd, 1H), 7.45 (dm, 1H), 7.35 (m, 1H), 5.03 (m, 1H), 3.89/3.33 (m+m, 2H). HRMS calculated for C₉H₈BrFO: 227.9586; found 227.95806 (M+).

### Example 138B rac-(1R,2S)-2-bromo-5-fluoro-2,3-dihydro-1H-inden-1-ol

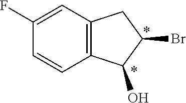 [SMILES: O[C@H]1c2ccc(F)cc2C[C@H]1Br]

Using General procedure 6 and Example 138A as the appropriate bromo-indan-1-one, Example 138B was obtained. ¹H NMR (500 MHz, DMSO-d₆) δ ppm: 7.32 (dd, 1H), 7.11 (dd, 1H), 7.05 (m, 1H), 5.86 (br s, 1H), 4.91 (m, 1H), 4.89 (m, 1H), 3.43/3.19 (dd+dd, 2H). HRMS calculated for C₉H₈BrFO: 229.9743; found 229.97360 (M+).

### Example 138C 2-bromo-6-fluoro-1H-indene

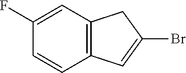 [SMILES: Fc1ccc2c(c1)CC(Br)=C2]

Using General procedure 7 and Example 138B as the appropriate indane, Example 138C was obtained. ¹H NMR (400 MHz, DMSO-d₆) δ ppm: 7.37 (dd, 1H), 7.29 (dd, 1H), 7.11 (dd, 1H), 7.10 (td, 1H), 3.73 (s, 2H). HRMS calculated for C₉H₈BrF: 211.9637; found 211.96294 (M+).

### Example 138D 2″-bromo-5″-fluorodispiro[[1,3]dioxolane-2,1′-cyclohexane-4′,1″-indene] and 2″-bromo-6″-fluorodispiro[[1,3]dioxolane-2,1′-cyclohexane-4′,1″-indene]

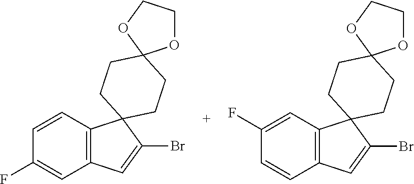 [SMILES: Fc1ccc2c(c1)C=C(Br)C21CCC2(CC1)OCCO2 ; Fc1ccc2c(c1)C1(CCC3(CC1)OCCO3)C(Br)=C2]

Using General procedure 8a and Example 138C as the appropriate indene, Example 138D was obtained as a mixture of regioisomers. ¹H NMR (400 MHz, DMSO-d₆) δ ppm: 7.69 (dd, 1H), 7.46 (dd, 1H), 7.14 (td, 1H), 7.04 (s, 1H), 3.96 (s, 4H), 2.09/1.88 (m, 4H), 2.09/1.2 (m, 4H) and 7.38 (dd, 1H), 7.21 (dd, 1H), 7.05 (s, 1H), 7.00 (td, 1H), 3.96 (s, 4H), 2.09/1.88 (m, 4H), 2.09/1.2 (m, 4H). HRMS calculated for C₁₆H₁₆BrFO₂: 338.0318; found 339.0388 and 339.0392 (M+H).

### Example 138E 2′-bromo-6′-fluorospiro[cyclohexane-1,1′-inden]-4-one

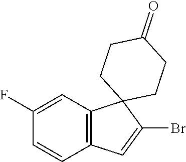 [SMILES: O=C1CCC2(CC1)C(Br)=Cc1ccc(F)cc12]

and

### Example 138F 2′-bromo-5′-fluorospiro[cyclohexane-1,1′-inden]-4-one

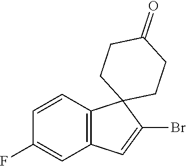 [SMILES: O=C1CCC2(CC1)C(Br)=Cc1cc(F)ccc12]

Using General procedure 9 and Example 138D as the appropriate ketal, a mixture of regioisomers was obtained. The regioisomers were separated via flash chromatography using hexane and acetone as eluents. The regioisomer eluting earlier was collected as Example 138E. ¹H NMR (400 MHz, DMSO-d₆) δ ppm: 7.88 (dd, 1H), 7.41 (dd, 1H), 7.17 (td, 1H), 7.11 (s, 1H), 2.94/2.49 (m, 4H), 2.20/1.64 (m, 4H). HRMS calculated for C₁₄H₁₂BrFO: 294.0056; found 294.00432 (M+).

The regioisomer eluting later was collected as Example 138F. ¹H NMR (500 MHz, DMSO-d₆) δ ppm: 7.95 (dd, 1H), 7.25 (dd, 1H), 7.11 (s, 1H), 7.02 (ddd, 1H), 2.90/2.48 (ddd+dm, 4H), 2.21/1.57 (td+dm, 4H). HRMS calculated for C₁₄H₁₂BrFO: 294.0056; found 294.00363 (M+).

### Example 138G 2″-bromo-5″-fluorodispiro[imidazolidine-4,1′-cyclohexane-4′,1″-indene]-2,5-dione

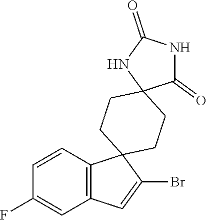 [SMILES: O=C1NC(=O)C2(CCC3(CC2)C(Br)=Cc2cc(F)ccc23)N1]

Using General procedure 14 and Example 138F as the appropriate ketone, Example 138G was obtained as a mixture of diastereoisomers. ¹H NMR (500 MHz, DMSO-d₆) δ ppm: 10.78/10.63 (br s/br s, 1H), 8.94/8.40 (s/s, 1H), 7.87/7.68 (dd/dd, 1H), 7.22/7.17 (dd/dd, 1H), 7.10/7.03 (td/td, 1H), 7.06/7.05 (s/s, 1H), 2.33 (td, 2H), 2.11 (td, 2H), 1.77 (d, 2H), 1.17 (d, 2H).

### Example 138H 4-amino-2′-bromo-5′-fluorospiro[cyclohexane-1,1′-indene]-4-carboxylic acid

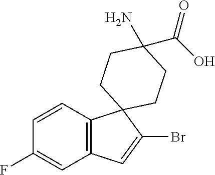 [SMILES: NC1(C(=O)O)CCC2(CC1)C(Br)=Cc1cc(F)ccc12]

Using General procedure 15 and Example 138G as the appropriate hydantoin, Example 138H was obtained as a mixture of diastereoisomers. ¹H NMR (400 MHz, DMSO-d₆) δ ppm: 8.05/8.02 (dd, 1H), 7.84 (br s, 2H), 7.22/7.18 (dd, 1H), 7.06/6.98 (td, 1H), 7.05/7.01 (s, 1H), 2.60/2.46/2.21/1.79 (m+m, 4H), 2.06/1.95/1.17/1.06 (m+m, 4H). HRMS calculated for C₁₅H₁₅BrFNO₂: 339.0270; found 340.0345 and 340.0344 (M+H).

### Example 138 (1s,4s)-2′-bromo-4-(3-chloroanilino)-5′-fluorospiro[cyclohexane-1,1′-indene]-4-carboxylic acid

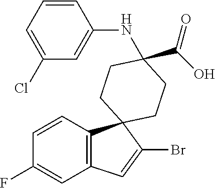 [SMILES: O=C(O)[C@]1(Nc2cccc(Cl)c2)CC[C@]2(CC1)C(Br)=Cc1cc(F)ccc12]

Using General procedure 16 and Example 138H as the appropriate amino acid and 1-chloro-3-iodo-benzene as the appropriate iodobenzene, a mixture of diastereoisomers was obtained. The diastereoisomers were separated by chiral chromatography. Column: AD, 50×500 mm, 20 μm, Eluents: 30:70 iPrOH/heptane+0.05% HCOOH. The diastereoisomer eluting earlier was further purified via prep RP-HPLC using 25 mM aq. NH₄HCO₃ solution and MeCN as eluents to give Example 138. ¹H NMR (500 MHz, DMSO-d₆) δ ppm: 12.87 (br s, 1H), 7.69 (dd, 1H), 7.22 (dd, 1H), 7.09 (t, 1H), 7.07 (m, 1H), 7.04 (s, 1H), 6.62 (t, 1H), 6.58 (dm, 1H), 6.55 (dm, 1H), 6.41 (br s, 1H), 2.35/2.25 (m+m, 4H), 2.19/0.97 (m+m, 4H). HRMS calculated for C₂₁H₁₈BrClFNO₂: 449.0193; found 450.0268 (M+H).

### Example 139

### Example 139A 2″-bromo-6″-fluorodispiro[imidazolidine-4,1′-cyclohexane-4′,1″-indene]-2,5-dione

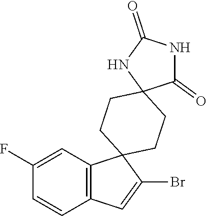 [SMILES: O=C1NC(=O)C2(CCC3(CC2)C(Br)=Cc2ccc(F)cc23)N1]

Using General procedure 14 and Example 138E as the appropriate ketone, Example 139A was obtained as a mixture of diastereoisomers. ¹H NMR (500 MHz, DMSO-d₆) δ ppm: 10.80/10.63 (br/br, 1H), 8.94/8.45 (s/s, 1H), 7.79/7.45 (dd/dd, 1H), 7.40/7.33 (dd/dd, 1H), 7.18/7.10 (td/td, 1H), 7.06/7.04 (s/s, 1H), 2.42-1.12 (m, 8H).

### Example 139B 4-amino-2′-bromo-6′-fluorospiro[cyclohexane-1,1′-indene]-4-carboxylic acid

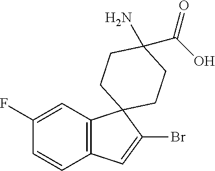 [SMILES: NC1(C(=O)O)CCC2(CC1)C(Br)=Cc1ccc(F)cc12]

Using General procedure 15 and Example 139A as the appropriate hydantoin, Example 139B was obtained as a mixture of diastereoisomers. ¹H NMR (500 MHz, DMSO-d₆) δ ppm: 8.02/7.90 (m/m, 1H), 7.37/7.33 (dd/dd, 1H), 7.14/7.10 (t/t, 1H), 7.02/6.98 (s/s, 1H), 2.68-1.00 (m, 8H).

### Example 139 (1s,4s)-2′-bromo-4-(3-chloroanilino)-6′-fluorospiro[cyclohexane-1,1′-indene]-4-carboxylic acid

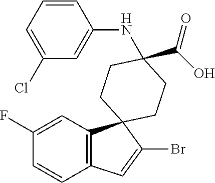 [SMILES: O=C(O)[C@]1(Nc2cccc(Cl)c2)CC[C@]2(CC1)C(Br)=Cc1ccc(F)cc12]

Using General procedure 16 and Example 139B as the appropriate amino acid and 1-chloro-3-iodo-benzene as the appropriate iodobenzene, a mixture of diastereoisomers was obtained. The diastereoisomers were separated via chiral chromatography. Column: AD, 100×500 mm, 20 μm, Eluents: iPrOH+HCOOH. The diastereoisomer eluting earlier was collected as Example 139. HRMS calculated for C₂₁H₁₈BrClFNO₂: 449.0193; found 450.0264 (M+H).

### Example 140

### Example 140A 2-bromo-5,6-dimethyl-2,3-dihydro-1H-inden-1-one

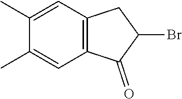

Using General procedure 5 and 5,6-dimethyl-2,3-dihydro-1H-inden-1-one as the appropriate indan-1-one, Example 140A was obtained. MS (EI, 70 eV) m/z (% relative intensity, [ion]): 91 (20), 115 (23), 131 (26), 159 (100), 238 (26, [M+]), 240 (25, [M+]).

### Example 140B rac-(1R,2S)-2-bromo-5,6-dimethyl-2,3-dihydro-1H-inden-1-ol

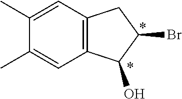

Using General procedure 6 and Example 140A as the appropriate bromo-indan-1-one, Example 140B was obtained. ¹H NMR (500 MHz, DMSO-d₆) δ ppm: 7.08 (s, 1H), 7.00 (s, 1H), 5.60 (d, 1H), 4.88-4.80 (m, 2H), 3.35-3.29 (dd, 1H), 3.11 (dd, 1H), 2.21 (s, 3H), 2.20 (s, 3H).

### Example 140C 2-bromo-5,6-dimethyl-1H-indene

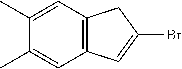

Using General procedure 7 and Example 140B as the appropriate indane, Example 140C was obtained. ¹H NMR (500 MHz, DMSO-d₆) δ ppm: 7.17 (s, 1H), 7.12 (s, 1H), 7.01 (m, 1H), 3.59 (s, 2H), 2.22 (s, 3H), 2.21 (s, 3H). MS (EI, 70 eV) m/z (% relative intensity, [ion]): 115 (23), 128 (77), 143 (100), 222 (18, [M+]), 224 (17, [M+]).

### Example 140D 2″-bromo-5″,6″-dimethyldispiro[[1,3]dioxolane-2,1′-cyclohexane-4′,1″-indene]

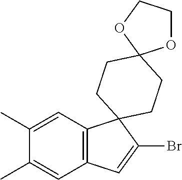 [SMILES: Cc1cc2c(cc1C)C1(CCC3(CC1)OCCO3)C(Br)=C2]

Using General procedure 8a and Example 140C as the appropriate indene, Example 140D was obtained. ¹H NMR (500 MHz, DMSO-d₆) δ ppm: 7.42 (s, 1H), 7.11 (s, 1H), 6.93 (s, 1H), 3.99-3.91 (m, 4H), 2.24 (s, 3H), 2.21 (s, 3H), 2.09/1.14 (m+m, 4H), 2.09/1.84 (m+m, 4H). HRMS calculated for C₁₈H₂₁BrO₂: 348.0725; found 349.0789 (M+H).

### Example 140E 2′-bromo-5′,6′-dimethylspiro[cyclohexane-1,1′-inden]-4-one

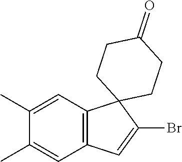 [SMILES: Cc1cc2c(cc1C)C1(CCC(=O)CC1)C(Br)=C2]

Using General procedure 9 and Example 140D as the appropriate ketal, Example 140E was obtained. MS (EI, 70 eV) m/z (% relative intensity, [ion]): 153 (27), 169 (38), 183 (100), 225 (50), 304 (40, [M+]), 306 (41, [M+]).

### Example 140F (1′s,1″s)-2″-bromo-5″,6″-dimethyldispiro[imidazolidine-4,1′-cyclohexane-4′,1″-indene]-2,5-dione

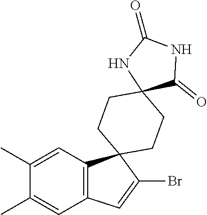 [SMILES: Cc1cc2c(cc1C)[C@]1(CC[C@@]3(CC1)NC(=O)NC3=O)C(Br)=C2]

Using General procedure 14 and Example 140E as the appropriate ketone, a mixture of diastereoisomers was obtained. Single diastereoisomer Example 140F was isolated via crystallization from CHCl₃. LRMS calculated for C₁₈H₁₉BrN₂O₂: 374.1; found 375.1 (M+H).

### Example 140G (1s,4s)-4-amino-2′-bromo-5′,6′-dimethylspiro[cyclohexane-1,1′-indene]-4-carboxylic acid

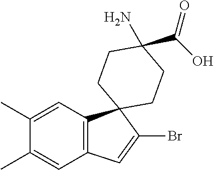 [SMILES: Cc1cc2c(cc1C)[C@]1(CC[C@@](N)(C(=O)O)CC1)C(Br)=C2]

Using General procedure 15 and Example 140F as the appropriate hydantoin, Example 140G was obtained. LRMS calculated for C₁₇H₂₀BrNO₂: 349.1; found 350.1 (M+H).

### Example 140 (1s,4s)-2′-bromo-4-(3-chloroanilino)-5′,6′-dimethylspiro[cyclohexane-1,1′-indene]-4-carboxylic acid

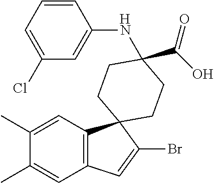

Using General procedure 16 and Example 140G as the appropriate amino acid and 1-chloro-3-iodo-benzene as the appropriate iodobenzene, Example 140 was obtained. ¹H NMR (500 MHz, DMSO-d₆) δ ppm: 7.48 (s, 1H), 7.13 (s, 1H), 7.07 (t, 1H), 6.93 (s, 1H), 6.62 (t, 1H), 6.55 (dm, 1H), 6.55 (dm, 1H), 6.38 (m, 1H), 2.39/2.22 (td+br d, 4H), 2.24 (s, 3H), 2.22 (s, 3H), 2.16/0.94 (td+br d, 4H). HRMS calculated for C₂₃H₂₃BrClNO₂: 459.0601; found 460.0675 (M+H).

### Example 141

### Example 141A 2-bromo-7-methyl-2,3-dihydro-1H-inden-1-one

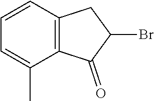

To a solution of 7-methyl-2,3-dihydro-1H-inden-1-one (1 g, 6.84 mmol, 1 eq) in DCM (12 mL) was added NBS (1.34 g, 7.52 mmol, 1.1 eq) and PTSA (130 mg, 0.68 mmol, 0.1 eq) and the mixture was heated at 55° C. for 2 h. The mixture was cooled to rt and partitioned between DCM and water. The organic phase was washed with brine, dried (MgSO₄) and concentrated in vacuo to afford a racemate, Example 141A, isolated as a yellow oil that was used directly in the subsequent step without further purification. LRMS calculated for C₁₀H₉BrO: 224; found: 225 (M+H). ¹H NMR (400 MHz, CDCl₃) δ ppm: 7.51 (t, J=7.5 Hz, 1H), 7.27-7.22 (m, 1H), 7.19-7.15 (m, 1H), 4.62 (dd, J=7.6, 3.3 Hz, 1H), 3.78 (dd, J=18.0, 7.6 Hz, 1H), 3.42-3.34 (m, 1H), 2.65 (s, 3H).

### Example 141B 2-bromo-7-methyl-2,3-dihydro-1H-inden-1-ol

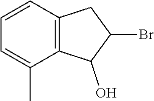

To a solution of Example 141A (1.54 g, 6.84 mmol, 1 eq) in MeOH (15 mL), cooled to 0° C., was added NaBH₄ (272 mg, 7.18 mmol, 1.05 eq) portionwise. The mixture was stirred at 0° C. for 1 h and then at rt for 1 h. The mixture was cooled to 0° C., quenched with the dropwise addition of water and then partitioned between DCM and water. The organic phase was washed with brine, dried (MgSO₄) and concentrated in vacuo to afford a single diastereoisomer, Example 141B, isolated as a cream solid (1.57 g) that was used directly in the subsequent step without further purification. ¹H NMR (400 MHz, DMSO-d₆) δ ppm: 7.16 (t, J=7.5 Hz, 1H), 7.07-7.00 (m, 2H), 5.45 (d, J=7.2 Hz, 1H), 4.93 (dd, J=7.2, 5.0 Hz, 1H), 4.71-4.64 (m, 1H), 3.33-3.16 (m, 2H), 2.34 (s, 3H).

### Example 141C 2-bromo-4-methyl-1H-indene

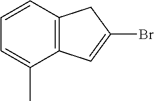

To a solution of Example 141B (1.57 g, 6.91 mmol, 1 eq) in toluene (100 mL) was added PTSA (723 mg, 3.8 mmol, 0.55 eq) portionwise and the mixture was refluxed for 3 h. The mixture was cooled to rt, quenched with sat. aq. NaHCO₃ solution and then partitioned between DCM and water. The organic phase was washed with brine, dried (MgSO₄) and concentrated in vacuo to afford Example 141C as a brown solid (1.34 g, 6.41 mmol, 93%) that was used directly in the subsequent step without further purification. ¹H NMR (400 MHz, CDCl₃) δ ppm: 7.23-7.18 (m, 1H), 7.10-7.02 (m, 3H), 3.63-3.59 (m, 2H), 2.38 (s, 3H).

### Example 141D 2″-bromo-4″-methyldispiro[[1,3]dioxolane-2,1′-cyclohexane-4′,1″-indene] and 2″-bromo-7″-methyldispiro[[1,3]dioxolane-2,1′-cyclohexane-4′,1″-indene]

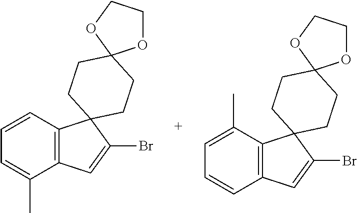 [SMILES: Cc1cccc2c1C=C(Br)C21CCC2(CC1)OCCO2 ; Cc1cccc2c1C1(CCC3(CC1)OCCO3)C(Br)=C2]

Using General procedure 8a and Example 141C (1.34 g, 6.41 mmol, 1 eq) as the appropriate indene, a 4:3 mixture of regioisomers, Example 141D, was isolated as a beige wax (1.6 g, 4.77 mmol, 75%). LRMS calculated for C₁₇H₁₉BrO₂: 334; found: 335 (M+H). ¹H NMR (400 MHz, CDCl₃) δ ppm: 7.55-6.74 (m, 4H), 4.08-4.00 (m, 4H), 2.87-2.63/2.29-2.16 (m, 4H), 2.56/2.37 (s, 3H), 2.02-1.91/1.78-1.70 (m, 2H), 1.61-1.53/1.40-1.28 (m, 2H).

### Example 141E 2′-bromo-4′-methylspiro[cyclohexane-1,1′-inden]-4-one

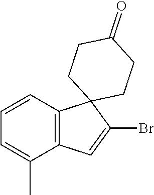 [SMILES: Cc1cccc2c1C=C(Br)C21CCC(=O)CC1]

and

### Example 141F 2′-bromo-7′-methylspiro[cyclohexane-1,1′-inden]-4-one

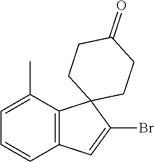 [SMILES: Cc1cccc2c1C1(CCC(=O)CC1)C(Br)=C2]

Using General procedure 9 and Example 141D (1.6 g, 4.77 mmol, 1 eq) as the appropriate ketal, a mixture of regioisomers were obtained. They were separated by automated flash chromatography (CombiFlash Rf, 40 g Gold RediSep™ silica cartridge) eluting with a gradient of 0-14% EtOAc in heptane. The regioisomer eluting earlier was collected as Example 141E, isolated as a white powder (622 mg, 2.14 mmol, 45%). LRMS calculated for C₁₅H₁₉BrO: 290; found: 291 (M+H). ¹H NMR (400 MHz, DMSO-d₆) δ ppm: 7.76-7.68 (m, 1H), 7.24 (d, J=0.6 Hz, 1H), 7.16-7.09 (m, 2H), 2.92 (ddd, J=15.7, 12.5, 6.1 Hz, 2H), 2.54-2.45 (m, 2H), 2.37 (s, 3H), 2.26-2.15 (m, 2H), 1.64-1.54 (m, 2H).

The regioisomer eluting later was collected as Example 141F, isolated as a beige powder (451 mg, 1.55 mmol, 32%). LRMS calculated for C₁₅H₁₉BrO: 290; found: 291 (M+H). ¹H NMR (400 MHz, DMSO-d₆) δ ppm: 7.18-7.12 (m, 2H), 7.04-6.97 (m, 2H), 3.04-2.93 (m, 2H), 2.78-2.68 (m, 2H), 2.59-2.50 (m, 2H), 2.48 (s, 3H), 1.85-1.76 (m, 2H).

### Example 141G 2″-bromo-4″-methyldispiro[imidazolidine-4,1′-cyclohexane-4′,1″-indene]-2,5-dione

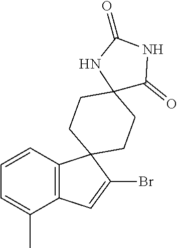 [SMILES: Cc1cccc2c1C=C(Br)C21CCC2(CC1)NC(=O)NC2=O]

To a solution of Example 141E (622 mg, 2.14 mmol, 1 eq) in EtOH (9 mL) and water (9 mL) was added NaCN (209 mg, 4.27 mmol, 2 eq) and (NH₄)₂CO₃ (821 mg, 8.54 mmol, 4 eq). The mixture was heated at 60° C. for 48 h. A mixture of diastereoisomers was formed. The mixture was cooled to rt and the precipitate was collected by filtration, washed with ice cold water (30 mL) and dried under vacuum to afford an 82:18 mixture of cis:trans isomers, Example 141G, isolated as a white solid (710 mg, 1.97 mmol, 92%). LRMS calculated for C₁₇H₁₇BrN₂O₂: 360; found: 361 (M+H). ¹H NMR (400 MHz, DMSO-d₆) δ ppm: 11.00-10.55 (br m, 1H), 8.96/8.46 (s, 1H), 7.72-7.66/7.58-7.49 (m, 1H), 7.22-7.05 (m, 3H), 2.45-2.26 (m, 5H), 2.18-2.04 (m, 2H), 2.02-1.91/1.81-1.70 (m, 2H), 1.63-1.52/1.22-1.11 (m, 2H).

### Example 141H (1s,4s)-4-amino-2′-bromo-4′-methylspiro[cyclohexane-1,1′-indene]-4-carboxylic acid

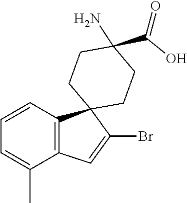 [SMILES: Cc1cccc2c1C=C(Br)[C@]21CC[C@](N)(C(=O)O)CC1]

Using General procedure 15 and Example 141G as the appropriate hydantoin, a single diastereoisomer, Example 141H was isolated as a cream powder. LRMS calculated for C₁₆H₁₈BrNO₂: 335; found: 336 (M+H). ¹H NMR (400 MHz, DMSO-d₆) δ ppm: 7.89-7.82 (m, 1H), 7.48 (br s, 3H), 7.15 (s, 1H), 7.14-7.05 (m, 2H), 2.69-2.57 (m, 2H), 2.35 (s, 3H), 2.12-2.00 (m, 2H), 1.76-1.66 (m, 2H), 1.09-0.99 (m, 2H).

### Example 141 (1s,4s)-2′-bromo-4-(3-chloroanilino)-4′-methylspiro[cyclohexane-1,1′-indene]-4-carboxylic acid

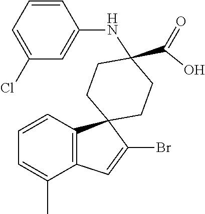

Using General procedure 16 and Example 141H as the appropriate amino acid and 3-chloroiodobenzene as the appropriate aryl iodide and 2-isobutyrylcyclohexanone instead of ethyl-2-oxocyclohexanecarboxylate afforded Example 141 as a beige powder. LRMS calculated for C₂₂H₂₁BrClNO₂: 445; found: 446 (M+H). ¹H NMR (400 MHz, DMSO-d₆) δ ppm: 12.85 (br s, 1H), 7.53 (d, J=7.0 Hz, 1H), 7.18-7.07 (m, 4H), 6.64-6.53 (m, 3H), 6.42 (br s, 1H), 2.46-2.32 (m, 5H), 2.29-2.14 (m, 4H), 1.03-0.94 (m, 2H).

### Example 142

### Example 142A 2″-methyldispiro[[1,3]dioxolane-2,1′-cyclohexane-4′,1″-indene]

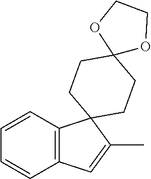 [SMILES: CC1=Cc2ccccc2C12CCC1(CC2)OCCO1]

Using General procedure 8a and 2-methyl-1H-indene as the appropriate indene and Preparation 1a, Example 142A was obtained as a brown oil. ¹H NMR (400 MHz, DMSO-d₆) δ ppm: 7.61 (d, J=7.6 Hz, 1H), 7.27-7.23 (m, 1H), 7.20 (td, J=7.3, 1.0 Hz, 1H), 7.08 (td, J=7.4, 1.5 Hz, 1H), 6.45-6.43 (m, 1H), 4.00-3.91 (m, 4H), 2.12 (td, J=13.2, 4.0 Hz, 2H), 2.01 (td, J=13.1, 3.8 Hz, 2H), 1.94 (d, J=1.5 Hz, 3H), 1.86-1.78 (m, 2H), 1.13-1.05 (m, 2H).

### Example 142B 2′-methylspiro[cyclohexane-1,1′-inden]-4-one

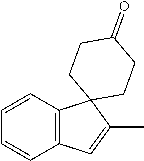 [SMILES: CC1=Cc2ccccc2C12CCC(=O)CC2]

Using General procedure 9 and Example 142A as the appropriate ketal, Example 142B was isolated as a beige solid. ¹H NMR (400 MHz, DMSO-d₆) δ ppm: 7.84 (d, J=7.6 Hz, 1H), 7.31-7.27 (m, 1H), 7.24 (td, J=7.3, 1.0 Hz, 1H), 7.11 (td, J=7.4, 1.5 Hz, 1H), 6.49-6.47 (m, 1H), 2.95 (ddd, J=15.6, 12.9, 6.1 Hz, 2H), 2.45-2.37 (m, 2H), 2.21 (td, J=13.3, 5.1 Hz, 2H), 1.97 (d, J=1.5 Hz, 3H), 1.47-1.39 (m, 2H).

### Example 142 (1r,4r)-4-(3-bromoanilino)-2′-methylspiro[cyclohexane-1,1′-indene]-4-carboxylic acid

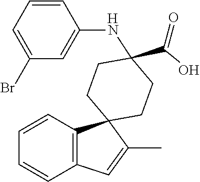

Using General Procedure 10 and Example 142B as the appropriate ketone and 3-bromoaniline as the appropriate aniline, a mixture of diastereoisomers was obtained. They were separated by chiral chromatography. Column: AD, 50 mm×500 mm, 20 m, Eluent: nPrOH+0.05% HCOOH. The diastereoisomer eluting earlier was collected as Example 142, isolated as a white solid. ¹H NMR (400 MHz, DMSO-d₆) δ ppm: 7.68 (d, 1H), 7.25 (dd, 1H), 7.20 (td, 1H), 7.09 (td, 1H), 7.00 (t, 1H), 6.80 (t, 1H), 6.65 (dm, 1H), 6.59 (dm, 1H), 6.42 (m, 1H), 6.26 (br s, 1H), 2.40/2.15 (td+d, 4H), 2.07/0.86 (td+d, 4H), 1.92 (d, 3H). HRMS calculated for C₂₂H₂₂BrNO₂: 411.0834; found: 412.0904 (M+H).

### Example 143

### Example 143A 2″-methyldispiro[[1,3]dioxolane-2,1′-cyclohexane-4′,1″-indene]

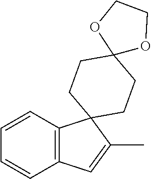 [SMILES: CC1=Cc2ccccc2C12CCC1(CC2)OCCO1]

Using General procedure 8a and 2-methyl-1H-indene as the appropriate indene, Example 143A was obtained. ¹H NMR (500 MHz, DMSO-d₆) δ ppm: 7.60 (d, 1H), 7.25 (d, 1H), 7.20 (td, 1H), 7.07 (td, 1H), 6.43 (m, 1H), 3.95 (s, 4H), 2.11/1.81 (td+dm, 4H), 2.00/1.08 (td+dm, 4H), 1.93 (d, 3H). HRMS calculated for C₁₇H₂₀O₂: 256.1463; found 256.14578 (M+).

### Example 143B 2′-methylspiro[cyclohexane-4,1′-indene]-1-one

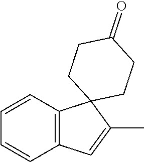 [SMILES: CC1=Cc2ccccc2C12CCC(=O)CC2]

Using General procedure 9 and Example 143A as the appropriate ketal, Example 143B was obtained. LRMS calculated for C₁₅H₁₆O: 212.1; found 213.0 (M+H).

### Example 143C 2″-methyldispiro[imidazolidine-4,1′-cyclohexane-4′,1″-indene]-2,5-dione

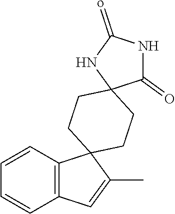

Using General procedure 14 and Example 143B as the appropriate ketone, Example 143C was obtained as a mixture of diastereoisomers. LRMS calculated for C₁₇H₁₈N₂O₂: 282.1; found 283.0 (M+H).

### Example 143D 4-amino-2′-methylspiro[cyclohexane-1,1′-indene]-4-carboxylic acid

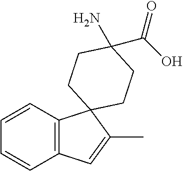 [SMILES: CC1=Cc2ccccc2C12CCC(N)(C(=O)O)CC2]

Using General procedure 15 and Example 143C as the appropriate hydantoin, Example 143D was obtained. ¹H NMR (500 MHz, DMSO-d₆) δ ppm: 7.95/7.90 (dm, 1H), 7.72 (br s, 3H), 7.27-6.99 (m, 3H), 6.40/6.39 (q, 1H), 2.71-0.81 (m, 8H), 1.96/1.94 (d, 3H). HRMS calculated for C₁₆H₁₉NO₂: 257.1416; found 258.1495 and 258.1493 (M+H).

### Example 143 (1r,4r)-4-(3-chloroanilino)-2′-methylspiro[cyclohexane-1,1′-indene]-4-carboxylic acid

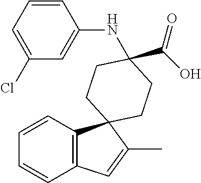 [SMILES: CC1=Cc2ccccc2[C@]12CC[C@@](Nc1cccc(Cl)c1)(C(=O)O)CC2]

Using General procedure 16 and Example 143D as the appropriate amino acid and 1-chloro-3-iodo-benzene as the appropriate iodobenzene, a mixture of diastereoisomers was obtained. The diastereoisomers were separated via chiral chromatography. Column: OJ, 100×500 mm, 20 μm, Eluents: EtOH/heptane+HCOOH. The diastereoisomer eluting earlier was collected as Example 143. ¹H NMR (500 MHz, DMSO-d₆) δ ppm: 12.83 (br s, 1H), 7.66 (dm, 1H), 7.25 (dm, 1H), 7.21 (m, 1H), 7.10 (m, 1H), 7.09 (t, 1H), 6.64 (t, 1H), 6.56 (dm, 1H), 6.56 (dm, 1H), 6.43 (q, 1H), 6.34 (br s, 1H), 2.40/2.19 (m+m, 4H), 2.09/0.88 (m+m, 4H), 1.92 (d, 3H). HRMS calculated for C₂₂H₂₂ClNO₂: 367.1339; found 368.1417 (M+H).

### Example 144 and Example 145

### Example 144A 2″-methyl-2″,3″-dihydrodispiro[[1,3]dioxolane-2,1′-cyclohexane-4′,1″-indene]

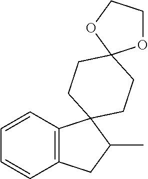 [SMILES: CC1Cc2ccccc2C12CCC1(CC2)OCCO1]

Using General procedure 19 and Example 142A as the appropriate indene afforded a racemate, Example 144A isolated as a colourless oil. ¹H NMR (400 MHz, DMSO-d₆) δ ppm: 7.23-7.09 (m, 4H), 3.98-3.84 (m, 4H), 3.12 (dd, J=15.5, 6.8 Hz, 1H), 2.49-2.36 (m, 2H), 1.95-1.84 (m, 1H), 1.80-1.51 (m, 6H), 1.48-1.39 (m, 1H), 0.86 (d, J=6.9 Hz, 3H).

### Example 144B 2′-methyl-2′,3′-dihydrospiro[cyclohexane-1,1′-inden]-4-one

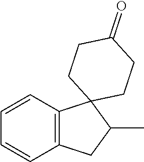 [SMILES: CC1Cc2ccccc2C12CCC(=O)CC2]

Using General procedure 9 and Example 144A as the appropriate ketal, a racemate, Example 144B was isolated as a colourless oil. ¹H NMR (400 MHz, DMSO-d₆) δ ppm: 7.33-7.26 (m, 1H), 7.26-7.11 (m, 3H), 3.18 (dd, J=15.7, 6.9 Hz, 1H), 2.70-2.57 (m, 2H), 2.51-2.27 (m, 4H), 2.12-2.03 (m, 1H), 1.97-1.87 (m, 2H), 1.75-1.64 (m, 1H), 0.97 (d, J=6.9 Hz, 3H).

### Example 144 (1r,2′R,4R)-4-(3-bromoanilino)-2′-methyl-2′,3′-dihydrospiro[cyclohexane-1,1′-indene]-4-carboxylic acid

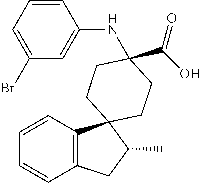 [SMILES: C[C@@H]1Cc2ccccc2[C@]12CC[C@@](Nc1cccc(Br)c1)(C(=O)O)CC2]

and

### Example 145 (1r,2′S,4S)-4-(3-bromoanilino)-2′-methyl-2′,3′-dihydrospiro[cyclohexane-1,1′-indene]-4-carboxylic acid

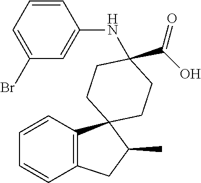 [SMILES: C[C@H]1Cc2ccccc2[C@]12CC[C@@](Nc1cccc(Br)c1)(C(=O)O)CC2]

Using General procedure 10 and Example 144B as the appropriate ketone and 3-bromoaniline as the appropriate aniline, a racemic mixture of diastereoisomers was obtained. They were separated by chiral chromatography. Column: Cellulose-1, 250 mm×21 mm. Eluent: 20 mM aq. NH₄HCO₂/0.1% TFA in MeCN. The diastereoisomer eluting firstly was collected as Example 144 and was obtained as an off-white foam. LRMS calculated for C₂₂H₂₄BrNO₂: 413; found: 414 (M+H). ¹H NMR (400 MHz, DMSO-d₆) δ ppm: 7.32-7.25 (m, 1H), 7.23-7.11 (m, 3H), 7.01 (t, J=8.1 Hz, 1H), 6.80 (t, J=2.1 Hz, 1H), 6.72-6.67 (m, 1H), 6.62-6.57 (m, 1H), 3.04 (dd, J=15.7, 7.0 Hz, 1H), 2.48-2.32 (m, 2H), 2.30-2.16 (m, 2H), 1.95-1.66 (m, 4H), 1.59-1.39 (m, 2H), 0.92 (d, J=6.9 Hz, 3H).

The diastereoisomer eluting fourthly was collected as Example 145 and was obtained as an off-white foam. LRMS calculated for C₂₂H₂₄BrNO₂: 413; found: 414 (M+H). ¹H NMR (400 MHz, DMSO-d₆) δ ppm: 7.32-7.25 (m, 1H), 7.23-7.11 (m, 3H), 7.01 (t, J=8.1 Hz, 1H), 6.80 (t, J=2.1 Hz, 1H), 6.72-6.67 (m, 1H), 6.62-6.57 (m, 1H), 3.04 (dd, J=15.7, 7.0 Hz, 1H), 2.48-2.32 (m, 2H), 2.30-2.16 (m, 2H), 1.95-1.66 (m, 4H), 1.59-1.39 (m, 2H), 0.92 (d, J=6.9 Hz, 3H).

### Example 146

### Example 146A 2″-ethyldispiro[[1,3]dioxolane-2,1′-cyclohexane-4′,1″-indene]

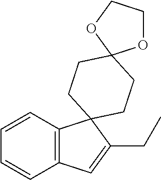 [SMILES: CCC1=Cc2ccccc2C12CCC1(CC2)OCCO1]

Using General procedure 8a and Preparation 1a and 2-ethyl-1H-indene as the appropriate indene, Example 146A was obtained as a brown oil. ¹H NMR (400 MHz, DMSO-d₆) δ ppm: 7.61 (dd, J=7.6, 0.9 Hz, 1H), 7.29 (dd, J=7.6, 1.2 Hz, 1H), 7.21 (td, J=7.4, 1.0 Hz, 1H), 7.08 (td, J=7.5, 1.3 Hz, 1H), 6.48-6.43 (m, 1H), 3.96 (s, 4H), 2.23 (qd, J=7.3, 1.9 Hz, 2H), 2.17-1.98 (m, 4H), 1.80 (d, J=12.2 Hz, 2H), 1.21 (t, J=7.3 Hz, 3H), 1.12-0.97 (m, 2H).

### Example 146B 2′-ethylspiro[cyclohexane-1,1′-inden]-4-one

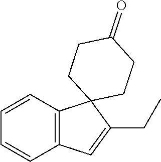 [SMILES: CCC1=Cc2ccccc2C12CCC(=O)CC2]

Using General procedure 9 and Example 146A as the appropriate ketal, Example 146B was isolated as a brown solid. ¹H NMR (400 MHz, DMSO-d₆) δ ppm: 7.83 (dd, J=7.7, 0.9 Hz, 1H), 7.33 (dt, J=7.3, 1.0 Hz, 1H), 7.25 (td, J=7.4, 1.0 Hz, 1H), 7.11 (td, J=7.5, 1.3 Hz, 1H), 6.53-6.42 (m, 1H), 2.95 (ddd, J=15.6, 13.1, 6.1 Hz, 2H), 2.45-2.34 (m, 2H), 2.32-2.17 (m, 4H), 1.48-1.34 (m, 2H), 1.23 (t, 3H).

### Example 146 (1s,4s)-4-(3-bromoanilino)-2′-ethylspiro[cyclohexane-1,1′-indene]-4-carboxylic acid

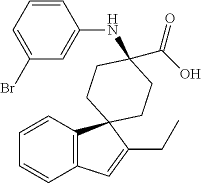 [SMILES: CCC1=Cc2ccccc2[C@]12CC[C@](Nc1cccc(Br)c1)(C(=O)O)CC2]

Using General procedure 10 and Example 146B as the appropriate ketone and 3-bromoaniline as the appropriate aniline, a single diastereoisomer, Example 146 was obtained as a cream solid. LRMS calculated for C₂₃H₂₄BrNO₂: 425; found: 426 (M+H). ¹H NMR (400 MHz, DMSO-d₆) δ ppm: 7.65 (d, J=7.5 Hz, 1H), 7.28 (dd, J=7.4, 1.2 Hz, 1H), 7.21 (td, J=7.4, 1.0 Hz, 1H), 7.10 (td, J=7.4, 1.4 Hz, 1H), 7.03 (t, J=8.0 Hz, 1H), 6.86 (t, J=2.1 Hz, 1H), 6.74-6.69 (m, 1H), 6.69-6.65 (m, 1H), 6.47-6.45 (m, 1H), 2.51-2.43 (m, 2H), 2.29-2.20 (m, 2H), 2.17-2.06 (m, 2H), 1.99-1.90 (m, 2H), 1.29-1.18 (m, 5H).

### Example 147

### Example 147A 2″-ethyl-2″,3″-dihydrodispiro[1,3-dioxolane-2,1′-cyclohexane-4′,1″-indene]

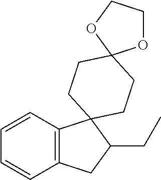 [SMILES: CCC1Cc2ccccc2C12CCC1(CC2)OCCO1]

Using General procedure 19 and Example 146A as the appropriate indene afforded a racemate, Example 147A isolated as a yellow oil. ¹H NMR (400 MHz, DMSO-d₆) δ ppm: 7.24-7.17 (m, 2H), 7.16-7.08 (m, 2H), 3.90 (m, 4H), 3.01 (dd, J=15.9, 7.0 Hz, 1H), 2.57 (dd, J=15.9, 4.4 Hz, 1H), 2.09 (m, 1H), 1.87-1.62 (m, 6H), 1.63-1.41 (m, 3H), 1.06-0.87 (m, 4H).

### Example 147B 2′-ethyl-2′,3′-dihydrospiro[cyclohexane-1,1′-inden]-4-one

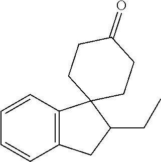 [SMILES: CCC1Cc2ccccc2C12CCC(=O)CC2]

Using General procedure 9 and Example 147A as the appropriate ketal afforded a racemate, Example 147B isolated as a yellow oil. ¹H NMR (400 MHz, DMSO-d₆) δ ppm: 7.38-7.32 (m, 1H), 7.26-7.20 (m, 1H), 7.19-7.11 (m, 2H), 3.07 (dd, J=15.8, 7.0 Hz, 1H), 2.68-2.51 (m, 3H), 2.39-2.30 (m, 2H), 2.25 (m, 1H), 2.03-1.88 (m, 3H), 1.75 (m, 1H), 1.66-1.54 (m, 1H), 1.15-1.03 (m, 1H), 0.96 (t, J=7.2 Hz, 3H).

### Example 147 4-(3-bromoanilino)-2′-ethyl-2′,3′-dihydrospiro[cyclohexane-1,1′-indene]-4-carboxylic acid

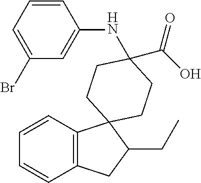 [SMILES: CCC1Cc2ccccc2C12CCC(Nc1cccc(Br)c1)(C(=O)O)CC2]

Using General procedure 10 and Example 147B as the appropriate ketone and 3-bromoaniline as the appropriate aniline, Example 147 was obtained as a racemic mixture of diastereoisomers, isolated as an orange foam. LRMS calculated for C₂₃H₂₆BrNO₂: 427; found: 428 (M+H). ¹H NMR (400 MHz, DMSO-d₆) δ ppm:12.67 (br s, 1H), 7.40-7.08 (m, 4H), 7.04-6.98 (m, 1H), 6.80/6.78 (t, J=2.1 Hz, 1H), 6.71-6.66 (m, 1H), 6.61-6.55 (m, 1H), 6.23 (br s, 1H), 3.07-2.93 (m, 1H), 2.64-2.49 (m, 1H), 2.46-1.31 (m, 10H), 1.20-0.87 (m, 4H).

### Example 148

### Example 148A 1H-inden-2-yl trifluoromethanesulfonate

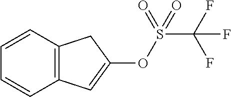 [SMILES: O=S(=O)(OC1=Cc2ccccc2C1)C(F)(F)F]

To a stirred solution of 2,3-dihydro-1H-inden-2-one (1.32 g, 10.0 mmol, 1 eq) in THE (30 mL) at −78° C. was added LDA (2.0 M, 6.5 mL, 13 mmol, 1.3 eq) dropwise. The reaction mixture was stirred at −78° C. for 2 h and then PhNTf₂ (5.36 g, 15 mmol, 1.5 eq) was added and the reaction was allowed to warm to rt and stirred for 18 h. The reaction mixture was diluted with EtOAc (10 mL) and the formed precipitate was removed by filtration. The filtrate was concentrated in vacuo. Purification by flash chromatography (40 g silica cartridge) eluting with PE afforded Example 148A as a colourless oil (2.0 g, 7.6 mmol, 75%). ¹H NMR (400 MHz, CDCl₃) δ ppm: 7.43-7.22 (m, 4H), 6.69 (s, 1H), 3.66 (s, 2H).

### Example 148B 2-[(1E)-prop-1-en-1-yl]-1H-indene

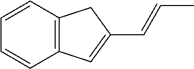

To a solution of Example 148A (950 mg, 3.60 mmol, 1.21 eq) in a mixture of 1,4-dioxane (20 mL) and water (5 mL) was added Na₂CO₃ (954 mg, 9.0 mmol, 3.0 eq), propen-1-ylboronic acid pinacol ester (500 mg, 2.98 mmol, 1 eq) and Pd(PPh₃)₄ (170 mg, 0.147 mmol, 0.05 eq). The reaction was heated at 95° C. for 18 h and then allowed to cool to rt before being diluted with EtOAc (30 mL). The solids were separated via filtration and the filtrate was concentrated in vacuo. Purification by flash chromatography (40 g silica cartridge) eluting with PE afforded Example 148B as a white solid (275 mg, 1.76 mmol, 59%). ¹H NMR (400 MHz, CDCl₃) δ ppm: 7.37 (d, J=7.4 Hz, 1H), 7.31-7.24 (m, 1H), 7.24-7.17 (m, 1H), 7.16-7.08 (m, 1H), 6.57 (s, 1H), 6.46 (d, J=15.7 Hz, 1H), 6.01-5.89 (m, 1H), 3.50 (s, 2H), 1.86 (d, J=6.8 Hz, 3H).

### Example 148C 2″-[(1E)-prop-1-en-1-yl]dispiro[[1,3]dioxolane-2,1′-cyclohexane-4′,1″-indene]

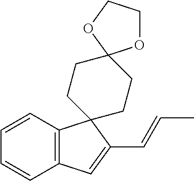 [SMILES: C/C=C/C1=Cc2ccccc2C12CCC1(CC2)OCCO1]

Using General procedure 8a with Preparation 1a and Example 148B as the appropriate indene, Example 148C was obtained as an orange oil. ¹H NMR (400 MHz, CDCl₃) δ ppm: 7.69 (d, J=7.6 Hz, 1H), 7.34-7.21 (m, 2H), 7.14-7.07 (m, 1H), 6.66 (s, 1H), 6.25-6.19 (m, 2H), 4.05 (s, 4H), 2.35-2.19 (m, 4H), 1.98-1.81 (m, 5H), 1.37-1.21 (m, 2H).

### Example 148D 2″-propyl-2″,3″-dihydrodispiro[[1,3]dioxolane-2,1′-cyclohexane-4′,1″-indene]

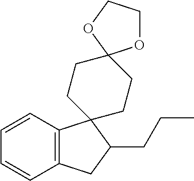 [SMILES: CCCC1Cc2ccccc2C12CCC1(CC2)OCCO1]

Using General procedure 19 and Example 148C as the appropriate indane, racemate Example 148D was obtained as a white solid. ¹H NMR (400 MHz, CDCl₃) δ ppm: 7.35-7.28 (m, 1H), 7.22-7.10 (m, 3H), 3.99 (s, 4H), 3.03 (dd, J=15.9, 7.1 Hz, 1H), 2.60 (dd, J=15.9, 5.1 Hz, 1H), 2.26-2.14 (m, 1H), 2.01-1.90 (m, 1H), 1.90-1.73 (m, 5H), 1.73-1.54 (m, 2H), 1.53-1.37 (m, 2H), 1.33-1.06 (m, 2H), 0.96-0.87 (m, 3H).

### Example 148E 2′-propyl-2′,3′-dihydrospiro[cyclohexane-1,1′-inden]-4-one

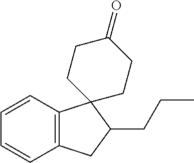 [SMILES: CCCC1Cc2ccccc2C12CCC(=O)CC2]

Using General procedure 9 and Example 148D as the appropriate ketal afforded a racemate, Example 148E isolated as an orange oil. ¹H NMR (400 MHz, CDCl₃) δ ppm: 7.34-7.12 (m, 4H), 3.09 (dd, J=15.8, 7.2 Hz, 1H), 2.73-2.47 (m, 4H), 2.44-2.25 (m, 2H), 2.14-1.99 (m, 2H), 1.99-1.87 (m, 2H), 1.58-1.41 (m, 2H), 1.40-1.20 (m, 2H), 0.99-0.91 (m, 3H).

### Example 148 (1r,4r)-4-(3-bromoanilino)-2′-propyl-2′,3′-dihydrospiro[cyclohexane-1,1′-indene]-4-carboxylic acid

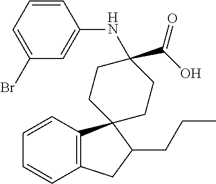

Using General procedure 10 and Example 148E as the appropriate ketone and 3-bromoaniline as the appropriate aniline afforded a racemic single diastereoisomer, Example 148 isolated as a white solid (60 mg, 0.14 mmol, 8%). LRMS calculated for C₂₄H₂₈BrNO₂: 441; found: 442 (M+H).

### Example 149

### Example 149A 2-(3-phenylpropyl)-1H-indene

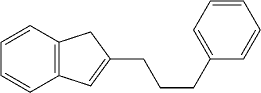 [SMILES: C1=C(CCCc2ccccc2)Cc2ccccc21]

To a solution of 2-bromo-1H-indene (200 mg, 1.03 mmol, 1 eq) and Ni(dppp)Cl₂ (5.6 mg, 0.01 mmol, 0.01 eq) in Et₂O (3 mL), cooled to −78° C., was added bromo(3-phenylpropyl)magnesium (2.26 mL, 0.5 M in THF, 1.13 mmol, 1.1 eq) slowly and the mixture was allowed to warm to rt and stirred for 2 h. The mixture was partitioned between Et₂O and 0.1 M aq. HCl solution and the organic phase was washed with brine, dried (MgSO₄) and concentrated in vacuo to afford Example 149A as a yellow oil (240 mg, 1.3 mmol, 100%). ¹H NMR (400 MHz, DMSO-d₆) δ ppm: 7.39-7.36 (m, 1H), 7.32-7.16 (m, 7H), 7.07 (td, J=7.4, 1.2 Hz, 1H), 6.58-6.56 (m, 1H), 3.35-3.33 (m, 2H), 2.67-2.62 (m, 2H), 2.51-2.46 (m, 2H), 1.94-1.85 (m, 2H).

### Example 149B 2″-(3-phenylpropyl)dispiro[[1,3]dioxolane-2,1′-cyclohexane-4′,1″-indene]

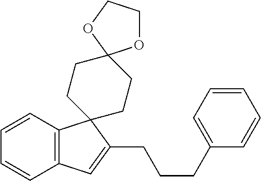 [SMILES: C1=C(CCCc2ccccc2)C2(CCC3(CC2)OCCO3)c2ccccc21]

Using General procedure 8a and Example 149A as the appropriate indene, Example 149B was obtained as a brown gum. ¹H NMR (400 MHz, DMSO-d₆) δ ppm: 7.61 (d, J=7.5 Hz, 1H), 7.34-7.16 (m, 7H), 7.08 (td, J=7.5, 1.3 Hz, 1H), 6.51 (d, J=1.7 Hz, 1H), 3.95 (s, 4H), 2.77-2.66 (m, 2H), 2.23 (ddd, J=8.3, 6.9, 1.8 Hz, 2H), 2.18-2.06 (m, 2H), 2.05-1.89 (m, 4H), 1.79 (d, J=12.8 Hz, 2H), 1.06 (d, J=12.8 Hz, 2H).

### Example 149C 2′-(3-phenylpropyl)spiro[cyclohexane-1,1′-inden]-4-one

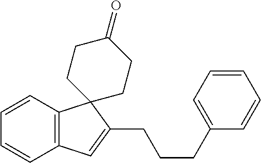 [SMILES: O=C1CCC2(CC1)C(CCCc1ccccc1)=Cc1ccccc12]

Using General procedure 9 and Example 149B as the appropriate ketone, Example 147B was isolated as a yellow oil. ¹H NMR (400 MHz, DMSO-d₆) δ ppm: 7.83 (d, J=7.5 Hz, 1H), 7.35-7.20 (m, 6H), 7.24-7.15 (m, 1H), 7.11 (td, J=7.5, 1.3 Hz, 1H), 6.55 (s, 1H), 2.94 (ddd, J=15.5, 13.2, 6.1 Hz, 2H), 2.75-2.66 (m, 2H), 2.38 (d, J=15.4 Hz, 2H), 2.33-2.14 (m, 4H), 2.01-1.88 (m, 2H), 1.46-1.34 (m, 2H).

### Example 149 (1s,4s)-4-(3-bromoanilino)-2′-(3-phenylpropyl)spiro[cyclohexane-1,1′-indene]-4-carboxylic acid

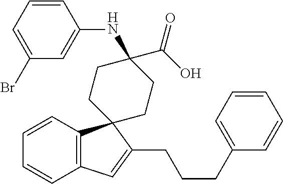 [SMILES: O=C(O)[C@]1(Nc2cccc(Br)c2)CC[C@@]2(CC1)C(CCCc1ccccc1)=Cc1ccccc12]

Using General Procedure 10 and Example 149C as the appropriate ketone and 3-bromoaniline as the appropriate aniline, a single diastereoisomer, Example 149 was obtained as a yellow solid. LRMS calculated for C₃₀H₃₀BrNO₂: 515; found: 516 (M+H). ¹H NMR (400 MHz, DMSO-d₆) δ ppm: 7.66 (d, J=7.5 Hz, 1H), 7.34-7.17 (m, 7H), 7.10 (td, J=7.4, 1.3 Hz, 1H), 7.03 (t, J=8.1 Hz, 1H), 6.86 (t, J=2.1 Hz, 1H), 6.74-6.70 (m, 1H), 6.70-6.65 (m, 1H), 6.52-6.50 (m, 1H), 2.74-2.67 (m, 2H), 2.51-2.44 (m, 2H), 2.29-2.22 (m, 2H), 2.15-2.05 (m, 2H), 2.01-1.90 (m, 4H), 1.27-1.18 (m, 2H).

### Example 150 and Example 151 Example 150A 2″-(3-phenylpropyl)-2″,3″-dihydrodispiro[[1,3]dioxolane-2,1′-cyclohexane-4′,1″-indene]

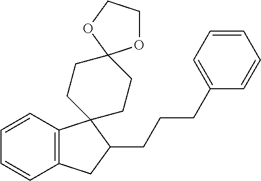 [SMILES: c1ccc(CCCC2Cc3ccccc3C23CCC2(CC3)OCCO2)cc1]

Using General procedure 19 and Example 149B as the appropriate indene in a mixture of THE and EtOH with 10% Pd/C, racemate Example 150A was obtained as a yellow gum (126 mg, 0.35 mmol, 90%). ¹H NMR (400 MHz, DMSO-d₆) δ ppm: 7.31-7.08 (m, 9H), 3.89 (s, 4H), 3.01 (dd, J=15.8, 7.0 Hz, 1H), 2.69-2.51 (m, 3H), 2.25-2.15 (m, 1H), 1.88-1.39 (m, 11H), 1.12-1.00 (m, 1H).

### Example 150B 2′-(3-phenylpropyl)-2′,3′-dihydrospiro[cyclohexane-1,1′-inden]-4-one

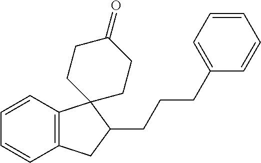 [SMILES: O=C1CCC2(CC1)c1ccccc1CC2CCCc1ccccc1]

To a solution of Example 150A (126 mg, 0.35 mmol, 1 eq) in THE (4 mL) was added 2 M aq. HCl solution (1.04 mL, 2.09 mmol, 6 eq) and the mixture was heated at 100° C. for 1 h under microwave irradiation. The mixture was cooled to rt and partitioned between EtOAc and sat. aq. NaHCO₃ solution. The aq. phase was extracted with further EtOAc and the combined organic extracts were washed with brine, dried (MgSO₄) and concentrated in vacuo to afford racemate Example 150B as a yellow oil (108 mg, 0.34 mmol, 98%). ¹H NMR (400 MHz, DMSO-d₆) δ ppm: 7.36-7.12 (m, 9H), 3.07 (dd, J=15.9, 7.0 Hz, 1H), 2.72-2.44 (m, 5H), 2.41-2.21 (m, 3H), 2.00-1.83 (m, 3H), 1.81-1.49 (m, 4H), 1.23-1.10 (m, 1H).

### Example 150 (1r,4r)-4-(3-bromoanilino)-2′-(3-phenylpropyl)-2′,3′-dihydrospiro[cyclohexane-1,1′-indene]-4-carboxylic acid, enantiomer 1

and

### Example 151 (1r,4r)-4-(3-bromoanilino)-2′-(3-phenylpropyl)-2′,3′-dihydrospiro[cyclohexane-1,1′-indene]-4-carboxylic acid, enantiomer 2

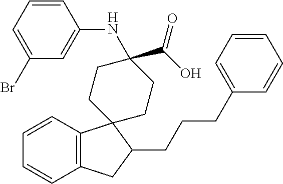

Using General Procedure 10 and Example 150B as the appropriate ketone and 3-bromoaniline as the appropriate amine, a racemic mixture of diastereoisomers was obtained. They were separated via chiral chromatography. Column: AD, 50×500 mm, 20 μm, Eluents: 20:80 EtOH/MeOH+0.05% HCOOH. The enantiomer eluting firstly was collected as Example 150. ¹H NMR (500 MHz, DMSO-d₆) δ ppm: 7.36 (d, 1H), 7.30-7.08 (m, 8H), 7.00 (t, 1H), 6.79 (br s, 1H), 6.67 (d, 1H), 6.56 (t, 1H), 2.97/2.52 (dd+dd, 2H), 2.64/2.57 (m+m, 2H), 2.45-1.10 (m, 12H), 2.00 (m, 1H). HRMS calculated for C₃₀H₃₂NO₂Br: 517.1616; found: 518.1697 (M+H).

The enantiomer eluting secondly was collected as Example 151. HRMS calculated for C₃₀H₃₂NO₂Br: 517.1616; found: 518.1695 (M+H).

### Example 152 (1r,4r)-4-(3-chloroanilino)-2′-[(1E)-3-cyclopentylprop-1-en-1-yl]spiro[cyclohexane-1,1′-indene]-4-carboxylic acid

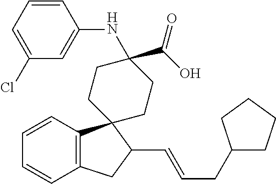

To an oven-dried microwave vial was added Example 75 (80 mg, 0.18 mmol, 1 eq), trans-3-(cyclopentyl)-1-propenylboronic acid pinacol ester (62 μL, 0.24 mmol, 1.3 eq) and K₃PO₄ (157 mg, 0.74 mmol, 4 eq) in 1,4-dioxane (2 mL) and water (0.4 mL). The mixture was sparged with N₂ before the addition of Pd(dppf)Cl₂×DCM (8 mg, 0.01 mmol, 0.05 eq), then the mixture was heated at 100° C. for 40 min under microwave irradiation. The reaction was partitioned between DCM and water, and the organic phase was washed with 1 M aq. HCl solution, brine, dried (PTFE phase separator) and concentrated in vacuo. Purification by automated flash chromatography (CombiFlash Rf, 4 g RediSep™ silica cartridge) eluting with a gradient of 0-60% EtOAc in heptane afforded Example 152 as a white powder (60 mg, 0.13 mmol, 70%). LRMS calculated for C₂₉H₃₂ClNO₂: 461; found: 462 (M+H). ¹H NMR (400 MHz, DMSO-d₆) δ ppm: 12.80 (br s, 1H), 7.70 (d, J=7.6 Hz, 1H), 7.34 (dd, J=7.5, 1.2 Hz, 1H), 7.25 (td, J=7.5, 0.9 Hz, 1H), 7.16 (td, J=7.5, 1.3 Hz, 1H), 7.09 (t, J=8.1 Hz, 1H), 6.73-6.70 (m, 2H), 6.61-6.54 (m, 2H), 6.45 (br s, 1H), 6.28-6.19 (m, 2H), 2.45-2.16 (m, 8H), 2.00-1.87 (m, 1H), 1.75-1.65 (m, 2H), 1.62-1.40 (m, 4H), 1.19-1.08 (m, 2H), 0.97-0.89 (m, 2H).

### Example 153 (1r,4r)-4-(3-chloroanilino)-2′-(3-cyclopentylpropyl)-2′,3′-dihydrospiro[cyclohexane-1,1′-indene]-4-carboxylic acid

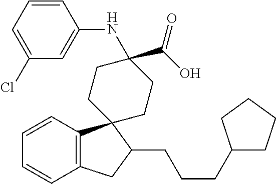

Using General procedure 19 and Example 152 as the appropriate indene afforded a racemate, Example 153, isolated as a white foam (11.8 mg, 0.03 mmol, 26%). LRMS calculated for C₂₉H₃₆ClNO₂: 465; found: 466 (M+H). ¹H NMR (400 MHz, DMSO-d₆) δ ppm: 12.75 (br s, 1H), 7.41-7.34 (m, 1H), 7.22-7.10 (m, 3H), 7.06 (t, J=8.1 Hz, 1H), 6.63 (t, J=2.1 Hz, 1H), 6.57-6.51 (m, 2H), 6.21 (br s, 1H), 2.96 (dd, J=15.7, 7.2 Hz, 1H), 2.58-2.49 (m, 1H), 2.46-2.36 (m, 1H), 2.15-2.04 (m, 1H), 2.01-1.00 (m, 22H).

### Example 154

### Example 154A (1r,4r)-2′-[(1E)-3-{[tert-butyl(dimethyl)silyl]oxy}prop-1-en-1-yl]-4-(3-chloroanilino)spiro[cyclohexane-1,1′-indene]-4-carboxylic acid

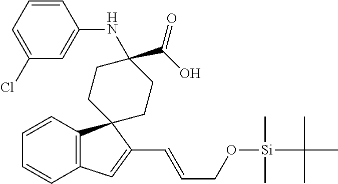 [SMILES: CC(C)(C)[Si](C)(C)OC/C=C/C1=Cc2ccccc2[C@]12CC[C@@](Nc1cccc(Cl)c1)(C(=O)O)CC2]

To an oven-dried microwave vial was added Example 75 (750 mg, 1.73 mmol, 1 eq), (E)-3-(tert-butyldimethylsilyloxy)propene-1-yl-boronic acid pinacol ester (682 μL, 2.08 mmol, 1.2 eq) and K₃PO₄ (1.47 g, 6.93 mmol, 4 eq) in 1,4-dioxane (15 mL) and water (4 mL). The mixture was sparged with N₂ before the addition of Pd(dppf)Cl₂×DCM (71 mg, 0.09 mmol, 0.05 eq), then heated at 110° C. for 40 min under microwave irradiation. The reaction was partitioned between DCM and water, and the organic phase was washed with 1 M aq. HCl solution, brine, dried (MgSO₄) and concentrated in vacuo. Purification by automated flash chromatography (CombiFlash Rf, 24 g RediSep™ silica cartridge) eluting with a gradient of 0-35% EtOAc in heptane afforded Example 154A as a white powder (613 mg, 1.17 mmol, 68%). ¹H NMR (400 MHz, DMSO-d₆) δ ppm: 12.79 (br s, 1H), 7.70 (d, J=7.5 Hz, 1H), 7.38-7.34 (m, 1H), 7.27 (td, J=7.4, 0.9 Hz, 1H), 7.17 (td, J=7.5, 1.3 Hz, 1H), 7.08 (t, J=8.1 Hz, 1H), 6.84 (s, 1H), 6.66 (t, J=2.1 Hz, 1H), 6.60-6.54 (m, 2H), 6.48-6.38 (m, 2H), 6.32 (dt, J=16.0, 4.4 Hz, 1H), 4.35-4.30 (m, 2H), 2.45-2.20 (m, 6H), 0.98-0.91 (m, 2H), 0.89 (s, 9H), 0.07 (s, 6H).

### Example 154 (1r,4r)-4-(3-chloroanilino)-2′-[(1E)-3-ethoxyprop-1-en-1-yl]spiro[cyclohexane-1,1′-indene]-4-carboxylic acid

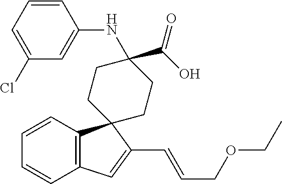 [SMILES: CCOC/C=C/C1=Cc2ccccc2[C@]12CC[C@@](Nc1cccc(Cl)c1)(C(=O)O)CC2]

To a solution of Example 154A (613 mg, 1.17 mmol, 1 eq) in EtOH (30 mL) was added cc. H₂SO₄ (0.75 mL) and the mixture was heated at reflux for 1.5 h. The mixture was cooled in an ice bath, neutralised with 2 M aq. NaOH solution and concentrated in vacuo. The residue was partitioned between EtOAc and water, the aq. phase was extracted with EtOAc, and the combined organic extract was washed with brine, dried (MgSO₄) and concentrated in vacuo. Purification by automated flash chromatography (CombiFlash Rf, 12 g RediSep™ silica cartridge) eluting with a gradient of 0-80% EtOAc in heptane afforded Example 154 as a cream powder (234 mg, 0.53 mmol, 46%). LRMS calculated for C₂₆H₂₈ClNO₃: 437; found: 438 (M+H). ¹H NMR (400 MHz, DMSO-d₆) δ ppm: 12.80 (br s, 1H), 7.71 (d, J=7.5 Hz, 1H), 7.37 (dd, J=7.4, 1.2 Hz, 1H), 7.27 (td, J=7.4, 0.9 Hz, 1H), 7.19 (td, J=7.5, 1.3 Hz, 1H), 7.10 (t, J=8.1 Hz, 1H), 6.85 (s, 1H), 6.68 (t, J=2.1 Hz, 1H), 6.62-6.56 (m, 2H), 6.52-6.38 (m, 2H), 6.30 (dt, J=16.3, 5.5 Hz, 1H), 4.11-4.06 (m, 2H), 3.48 (q, J=7.0 Hz, 2H), 2.45-2.21 (m, 6H), 1.15 (t, J=7.0 Hz, 3H), 0.99-0.90 (m, 2H).

### Example 155 (1r,4r)-4-(3-chloroanilino)-2′-[3-(4-methoxyphenyl)propyl]spiro[cyclohexane-1,1′-indene]-4-carboxylic acid

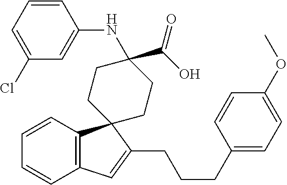 [SMILES: COc1ccc(CCCC2=Cc3ccccc3[C@]23CC[C@@](Nc2cccc(Cl)c2)(C(=O)O)CC3)cc1]

Using General procedure 27a and Example 75 as the appropriate 2-bromo-indene derivative and 3-(4-methoxyphenyl)propylzinc chloride as the appropriate zinc reagent, Example 155 was obtained as a white powder. LRMS calculated for C₃₁H₃₂ClNO₃: 501; found: 502 (M+H). ¹H NMR (400 MHz, DMSO-d₆) δ ppm: 12.82 (br s, 1H), 7.66 (d, J=7.6 Hz, 1H), 7.30-7.27 (m, 1H), 7.25-7.20 (m, 1H), 7.18-7.08 (m, 4H), 6.87-6.82 (m, 2H), 6.66 (t, J=2.1 Hz, 1H), 6.61-6.55 (m, 2H), 6.51-6.48 (m, 1H), 6.32 (br s, 1H), 3.72 (s, 3H), 2.63 (t, J=7.7 Hz, 2H), 2.45-2.34 (m, 2H), 2.28-2.05 (m, 6H), 1.95-1.85 (m, 2H), 0.93-0.84 (m, 2H).

### Example 156 (1r,4r)-4-(3-chloroanilino)-2′-(3-ethoxypropyl)-2′,3′-dihydrospiro[cyclohexane-1,1′-indene]-4-carboxylic acid

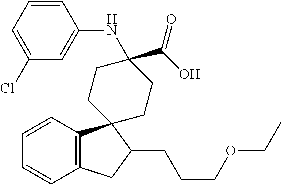

Using General procedure 19 and Example 154 as the appropriate indene afforded a racemate, Example 156 isolated as an off-white powder. LRMS calculated for C₂₆H₃₂ClNO₃: 465; found: 466 (M+H). ¹H NMR (400 MHz, DMSO-d₆) δ ppm: 7.40-7.33 (m, 1H), 7.23-7.10 (m, 3H), 7.06 (t, J=8.1 Hz, 1H), 6.63 (t, J=2.1 Hz, 1H), 6.58-6.51 (m, 2H), 6.23 (br s, 1H), 3.44-3.35 (m, 4H), 2.98 (dd, J=15.6, 7.2 Hz, 1H), 2.57-2.50 (m, 1H), 2.47-2.36 (m, 1H), 2.17-2.06 (m, 1H), 2.03-1.83 (m, 4H), 1.78-1.33 (m, 6H), 1.21-1.11 (m, 1H), 1.09 (t, J=7.0 Hz, 3H).

### Example 157 (1r,4r)-4-(3-chloroanilino)-2′-[3-(4-chlorophenyl)propyl]spiro[cyclohexane-1,1′-indene]-4-carboxylic acid

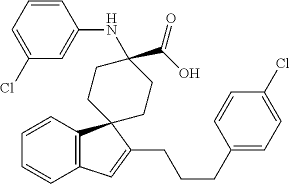 [SMILES: O=C(O)[C@]1(Nc2cccc(Cl)c2)CC[C@]2(CC1)C(CCCc1ccc(Cl)cc1)=Cc1ccccc12]

To an oven-dried microwave vial was added Example 75 (150 mg, 0.35 mmol, 1 eq) and AtaPhos (10 mg, 13.6 μmol, 0.04 eq) in THF (2 mL). 1-Methylimidazole (55 μL, 0.69 mmol, 2 eq) was added followed by 3-(4-chlorophenyl)propylzinc chloride solution (0.5 M in THF, 2.72 mL, 1.36 mmol, 4 eq) and then heated at 120° C. for 2 h under microwave irradiation. The reaction was partitioned between DCM and water, and the organic phase was washed with 1 M aq. HCl solution, brine, dried (PTFE phase separator) and concentrated in vacuo. Purification by reverse phase automated flash chromatography (CombiFlash Rf, C18 13 g RediSep column) eluting with a gradient of 15-100% MeCN in water afforded Example 157 as a white powder (54 mg, 0.11 mmol, 31%). LRMS calculated for C₃₀H₂₉Cl₂NO₂: 505; found: 506 (M+H). ¹H NMR (400 MHz, DMSO-d₆) δ ppm: 12.79 (br s, 1H), 7.66 (d, J=7.5 Hz, 1H), 7.36-7.31 (m, 2H), 7.31-7.25 (m, 3H), 7.25-7.20 (m, 1H), 7.16-7.06 (m, 2H), 6.65 (t, J=2.1 Hz, 1H), 6.61-6.53 (m, 2H), 6.51 (d, J=1.6 Hz, 1H), 6.33 (br s, 1H), 2.74-2.65 (m, 2H), 2.45-2.34 (m, 2H), 2.28-2.16 (m, 4H), 2.15-2.05 (m, 2H), 1.93 (q, J=7.6 Hz, 2H), 0.88 (d, J=13.4 Hz, 2H).

### Example 158 (1r,4r)-2′-[(E)-2-([1,1′-biphenyl]-4-yl)ethenyl]-4-(3-chloroanilino)spiro[cyclohexane-1,1′-indene]-4-carboxylic acid

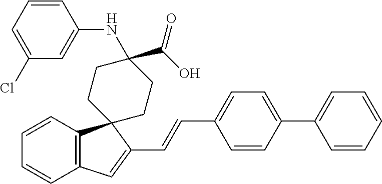 [SMILES: O=C(O)[C@]1(Nc2cccc(Cl)c2)CC[C@]2(CC1)C(/C=C/c1ccc(-c3ccccc3)cc1)=Cc1ccccc12]

Using General procedure 18 and Preparation 6a as the appropriate 2-bromo-indene derivative and [(E)-2-([1,1′-biphenyl]-4-yl)ethenyl]boronic acid as the appropriate boronic acid, Example 158 was obtained. ¹H NMR (500 MHz, DMSO-d₆) δ ppm: 7.77 (dm, 1H), 7.76-7.65 (m, 4H), 7.73 (m, 2H), 7.48 (m, 2H), 7.42 (dm, 1H), 7.37 (m, 1H), 7.29 (m, 1H), 7.21 (m, 1H), 7.19-7.14 (m, 2H), 7.12 (t, 1H), 7.03 (s, 1H), 6.84 (t, 1H), 6.70 (dm, 1H), 6.56 (dm, 1H), 2.47/1.00 (m+m, 4H), 2.45/2.27 (m+m, 4H). HRMS calculated for C₃₅H₃₀NO₂Cl: 531.1965; found: 532.2054 (M+H).

### Example 159 (1r,4r)-4-(3-chloroanilino)-2′-[(1E)-prop-1-en-1-yl]spiro[cyclohexane-1,1′-indene]-4-carboxylic acid

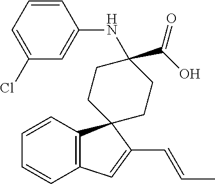 [SMILES: C/C=C/C1=Cc2ccccc2[C@]12CC[C@@](Nc1cccc(Cl)c1)(C(=O)O)CC2]

Using General procedure 18 and Preparation 6a as the appropriate 2-bromo-indene derivative and (1E)-prop-1-en-1-ylboronic acid as the appropriate boronic acid, Example 159 was obtained. ¹H NMR (500 MHz, DMSO-d₆) δ ppm: 12.82 (br s, 1H), 7.68 (dm, 1H), 7.32 (dm, 1H), 7.24 (m, 1H), 7.14 (m, 1H), 7.10 (t, 1H), 6.72 (s, 1H), 6.66 (t, 1H), 6.59 (dm, 1H), 6.58 (dm, 1H), 6.43 (br s, 1H), 6.31-6.16 (m, 2H), 2.38/2.22 (m+m, 4H), 2.28/0.92 (m+m, 4H), 1.87 (d, 3H). HRMS calculated for C₂₄H₂₄NO₂Cl: 393.1496; found: 394.1579 (M+H).

### Example 160 (1r,4r)-4-(3-chloroanilino)-2′-(4-phenoxybutyl)spiro[cyclohexane-1,1′-indene]-4-carboxylic acid

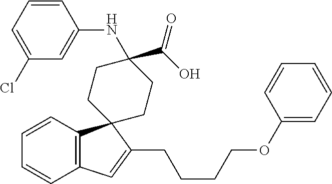 [SMILES: O=C(O)[C@]1(Nc2cccc(Cl)c2)CC[C@]2(CC1)C(CCCCOc1ccccc1)=Cc1ccccc12]

To an oven-dried microwave vial was added Example 75 (200 mg, 0.46 mmol, 1 eq) and Ataphos (7 mg, 0.01 mmol, 0.02 eq) in THE (3 mL), followed by 1-methylimidazole (74 μL, 0.92 mmol, 2 eq) and 4-phenoxybutylzinc bromide solution (1.85 mL, 0.5 M in MeOH, 0.92 mmol, 2 eq). The mixture was sparged with N₂ and heated at 120° C. for 1 h under microwave irradiation. The reaction was partitioned between DCM and water, and the organic phase was washed with 1 M aq. HCl solution, brine, dried (PTFE phase separator) and concentrated in vacuo. Purification by reverse phase automated flash chromatography (CombiFlash Rf, C18 13 g RediSep column) eluting with a gradient of 15-100% MeCN in water afforded Example 160 as an off-white powder (79 mg, 0.16 mmol, 34%). LRMS calculated for C₃₁H₃₂ClNO₃: 501; found: 502 (M+H). ¹H NMR (400 MHz, DMSO-d₆) δ ppm: 12.78 (br s, 1H), 7.66 (d, J=7.6 Hz, 1H), 7.33-7.18 (m, 4H), 7.16-7.05 (m, 2H), 6.98-6.85 (m, 3H), 6.66 (t, J=2.1 Hz, 1H), 6.58 (dd, J=8.1, 2.1 Hz, 2H), 6.48 (s, 1H), 6.36 (br s, 1H), 4.03 (t, J=5.8 Hz, 2H), 2.47-2.35 (m, 2H), 2.29 (t, J=6.8 Hz, 2H), 2.24-2.08 (m, 4H), 1.90-1.72 (m, 4H), 0.90 (d, J=13.5 Hz, 2H).

### Example 161 (1r,4r)-4-(3-chloroanilino)-2′-[3-(4-methoxyphenyl)propyl]-2′,3′-dihydrospiro[cyclohexane-1,1′-indene]-4-carboxylic acid

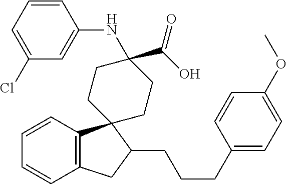

Using General procedure 19 and Example 155 as the appropriate indene afforded a racemate, Example 161, isolated as a white powder. LRMS calculated for C₃₁H₃₄ClNO₃: 503; found: 504 (M+H). ¹H NMR (400 MHz, DMSO-d₆) δ ppm: 7.39-7.32 (m, 1H), 7.21-7.08 (m, 5H), 7.03 (t, J=8.1 Hz, 1H), 6.84-6.79 (m, 2H), 6.66 (t, J=2.3 Hz, 1H), 6.59-6.54 (m, 1H), 6.52-6.48 (m, 1H), 6.09 (br s, 1H), 3.70 (s, 3H), 2.97 (dd, J=15.7, 7.3 Hz, 1H), 2.66-2.49 (m, 3H), 2.45-2.33 (m, 1H), 2.18-1.74 (m, 5H), 1.74-1.61 (m, 2H), 1.60-1.36 (m, 4H), 1.21-1.09 (m, 1H).

### Example 162 (1r,4r)-4-(3-chloroanilino)-2′-[3-(4-chlorophenyl)propyl]-2′,3′-dihydrospiro[cyclohexane-1,1′-indene]-4-carboxylic acid

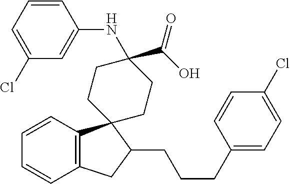

Using General procedure 19 and Example 157 as the appropriate indene afforded a racemate, Example 162 isolated as a white powder. LRMS calculated for C₃₀H₃₁Cl₂NO₂: 507; found: 508 (M+H). ¹H NMR 400 MHz, DMSO-d₆) δ ppm: 7.39-7.34 (m, 1H), 7.33-7.29 (m, 2H), 7.26-7.22 (m, 2H), 7.21-7.10 (m, 3H), 7.06 (t, J=8.1 Hz, 1H), 6.63 (t, J=2.1 Hz, 1H), 6.57-6.51 (m, 2H), 2.98 (dd, J=15.5, 7.2 Hz, 1H), 2.67-2.49 (m, 3H), 2.46-2.35 (m, 1H), 2.15-1.79 (m, 5H), 1.77-1.06 (m, 7H).

### Example 163

### Example 163A [(1H-inden-2-yl)ethynyl](trimethyl)silane

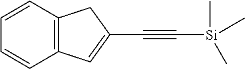

To an oven-dried flask was added 2-bromo-1H-indene (1.02 g, 5.23 mmol, 1 eq), DIPA (1.5 mL, 10.7 mmol, 2.04 eq), CuI (20 mg, 0.1 mmol, 0.02 eq), PPh₃ (55 mg, 0.21 mmol, 0.04 eq) and Pd(dppf)Cl₂ (73 mg, 0.1 mmol, 0.02 eq) in toluene (10 mL). The mixture was sparged with N₂, ethynyl(trimethyl)silane (0.87 mL, 6.28 mmol, 1.2 eq) was added and then heated at 80° C. for 18 h. The reaction was partitioned between EtOAc and water, and the organic phase was washed with brine, dried (MgSO₄) and concentrated in vacuo. Purification by flash chromatography (20 g silica cartridge) eluting with a gradient of 0-10% EtOAc in heptane afforded Example 163A as a cream solid (1.11 g, 5.23 mmol, 100%). ¹H NMR 400 MHz, DMSO-d₆) δ ppm: 7.47-7.39 (m, 2H), 7.32-7.20 (m, 3H), 3.60-3.52 (m, 2H), 0.22 (s, 9H).

### Example 163B 2″-ethynyldispiro[[1,3]dioxolane-2,1′-cyclohexane-4′,1″-indene]

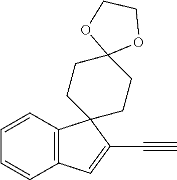 [SMILES: C#CC1=Cc2ccccc2C12CCC1(CC2)OCCO1]

Using General procedure 8a and Example 163A as the appropriate indene, Example 163B was obtained as a yellow solid. LRMS calculated for C₁₈H₁₈O₂: 266; found: 267 (M+H).

### Example 163C 4-[(dispiro[[1,3]dioxolane-2,1′-cyclohexane-4′,1″-inden]-2″-yl)ethynyl]-1H-indole

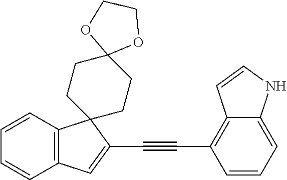 [SMILES: C(#Cc1cccc2[nH]ccc12)C1=Cc2ccccc2C12CCC1(CC2)OCCO1]

To an oven-dried flask was added Example 163B (200 mg, 0.75 mmol, 1 eq), 4-bromo-1H-indole (113 μL, 0.9 mmol, 1.2 eq), DIPA (228 μL, 1.62 mmol, 2.16 eq), CuI (3 mg, 0.02 mmol, 0.02 eq), PPh₃ (8 mg, 0.03 mmol, 0.04 eq) and Pd(PPh₃)₂Cl₂ (11 mg, 0.02 mmol, 0.02 eq) in toluene (6 mL). The mixture was sparged with N₂ and heated at 80° C. for 18 h. The reaction was partitioned between DCM and water, and the organic phase was washed with brine, dried (MgSO₄) and concentrated in vacuo. Purification by flash chromatography (10 g silica cartridge) eluting with a gradient of 0-20% EtOAc in heptane afforded Example 163C as a yellow powder (127 mg, 0.33 mmol, 44%). LRMS calculated for C₂₆H₂₃NO₂: 381; found: 382 (M+H).

### Example 163D 4-[2-(2″,3″-dihydrodispiro[[1,3]dioxolane-2,1′-cyclohexane-4′,1″-inden]-2″-yl)ethyl]-1H-indole

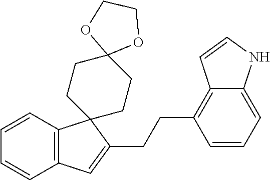 [SMILES: C1=C(CCc2cccc3[nH]ccc23)C2(CCC3(CC2)OCCO3)c2ccccc21]

Using General procedure 19 and Example 163C as the appropriate indene afforded a racemate, Example 163D isolated as a colourless solid. LRMS calculated for C₂₆H₂₉NO₂: 387; found: 388 (M+H).

### Example 163E 2′-[2-(1H-indol-4-yl)ethyl]-2′,3′-dihydrospiro[cyclohexane-1,1′-inden]-4-one

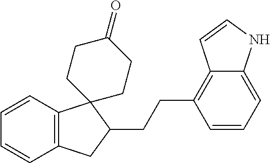 [SMILES: O=C1CCC2(CC1)c1ccccc1CC2CCc1cccc2[nH]ccc12]

Using General procedure 9 and Example 163D as the appropriate ketal afforded a racemate, Example 163E isolated as a pink gum. LRMS calculated for C₂₄H₂₅NO: 343; found: 344 (M+H).

### Example 163 (1r,4r)-4-(3-bromoanilino)-2′-[2-(1H-indol-4-yl)ethyl]-2′,3′-dihydrospiro[cyclohexane-1,1′-indene]-4-carboxylic acid

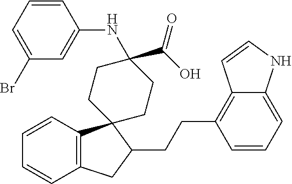

Using General procedure 10 and Example 163E as the appropriate ketone and 3-bromoaniline as the appropriate aniline, a racemic single diastereoisomer, Example 163 was isolated as an off-white powder. LRMS calculated for C₃₁H₃₁BrN₂O₂: 542; found: 543 (M+H). ¹H NMR (400 MHz, DMSO-d₆) δ ppm: 12.58 (br s, 1H), 11.03 (s, 1H), 7.31-7.11 (m, 6H), 7.03-6.95 (m, 2H), 6.83-6.77 (m, 2H), 6.70-6.65 (m, 1H), 6.60-6.56 (m, 1H), 6.43-6.40 (m, 1H), 6.22 (br s, 1H), 3.14 (dd, J=16.0, 7.0 Hz, 1H), 3.02-2.91 (m, 1H), 2.87-2.75 (m, 2H), 2.38-2.29 (m, 1H), 2.15-1.81 (m, 6H), 1.62-1.45 (m, 3H), 1.42-1.30 (m, 1H).

### Example 164

### Example 164A 3-(2-{dispiro[1,3-dioxolane-2,1′-cyclohexane-4′,1″-inden]-2″-yl}ethynyl)pyridine

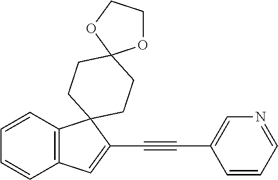 [SMILES: C(#Cc1cccnc1)C1=Cc2ccccc2C12CCC1(CC2)OCCO1]

To an oven-dried flask was added Example 163B (200 mg, 0.75 mmol, 1 eq), 3-bromopyridine (88 μL, 0.9 mmol, 1.2 eq), DIPA (0.24 mL, 1.71 mmol, 2.27 eq), CuI (3 mg, 0.02 mmol, 0.02 eq), PPh₃ (8 mg, 0.03 mmol, 0.04 eq) and Pd(PPh₃)₂Cl₂ (11 mg, 0.02 mmol, 0.02 eq) in toluene (6 mL). The mixture was sparged with N₂ and heated at 80° C. for 18 h. The reaction was partitioned between DCM and water, and the organic phase was washed with brine, dried (MgSO₄) and concentrated in vacuo. Purification by flash chromatography (10 g silica cartridge) eluting with a gradient of 0-20% EtOAc in heptane afforded Example 164A as a yellow solid (228 mg, 0.66 mmol, 88%). LRMS calculated for C₂₃H₂₁NO₂: 343; found: 344 (M+H).

### Example 164B 3-[2-(2″,3″-dihydrodispiro[[1,3]dioxolane-2,1′-cyclohexane-4′,1″-inden]-2″-yl)ethyl]pyridine

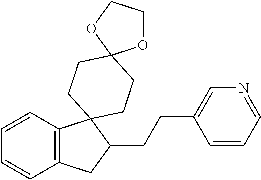 [SMILES: c1cncc(CCC2Cc3ccccc3C23CCC2(CC3)OCCO2)c1]

Using General procedure 19 and Example 164A as the appropriate indene afforded a racemate, Example 164B isolated as a yellow gum. LRMS calculated for C₂₃H₂₇NO₂: 349; found: 350 (M+H).

### Example 164C 2′-[2-(pyridin-3-yl)ethyl]-2′,3′-dihydrospiro[cyclohexane-1,1′-inden]-4-one

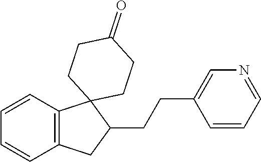 [SMILES: O=C1CCC2(CC1)c1ccccc1CC2CCc1cccnc1]

Using General procedure 9 and Example 164B as the appropriate ketal afforded a racemate, Example 164C isolated as a yellow gum. LRMS calculated for C₂₁H₂₃NO: 305; found: 306 (M+H).

### Example 164 (1r,4r)-4-(3-bromoanilino)-2′-[2-(pyridin-3-yl)ethyl]-2′,3′-dihydrospiro[cyclohexane-1,1′-indene]-4-carboxylic acid

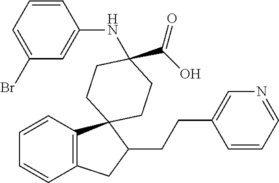

Using General procedure 10 and Example 164C as the appropriate ketone and 3-bromoaniline as the appropriate aniline, a racemic single diastereoisomer, Example 164 was isolated as a white powder. LRMS calculated for C₂₈H₂₉BrN₂O₂: 504; found: 505 (M+H). ¹H NMR (400 MHz, DMSO-d₆) δ ppm: 8.48 (d, J=2.2 Hz, 1H), 8.40 (dd, J=4.8, 1.7 Hz, 1H), 7.66 (dt, J=7.8, 2.1 Hz, 1H), 7.42-7.36 (m, 1H), 7.31 (dd, J=7.9, 4.7 Hz, 1H), 7.24-7.11 (m, 3H), 6.96 (t, J=8.0 Hz, 1H), 6.78 (t, J=2.0 Hz, 1H), 6.65-6.60 (m, 1H), 6.60-6.54 (m, 1H), 3.03 (dd, J=15.6, 7.2 Hz, 1H), 2.84-2.74 (m, 1H), 2.68-2.54 (m, 2H), 2.44-2.34 (m, 1H), 2.11-1.64 (m, 7H), 1.56-1.29 (m, 3H).

### Example 165

### Example 165A 2-(3-phenoxyprop-1-yn-1-yl)-1H-indene

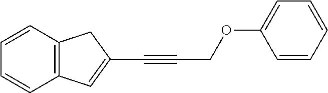 [SMILES: C(#CC1=Cc2ccccc2C1)COc1ccccc1]

To an oven-dried flask was added 2-bromo-1H-indene (308 mg, 1.58 mmol, 1 eq), DIPA (0.5 mL) CuI (6 mg, 0.03 mmol, 0.02 eq), PPh₃ (17 mg, 0.06 mmol, 0.04 eq) and Pd(PPh₃)₂Cl₂ (22 mg, 0.03 mmol, 0.02 eq) in toluene (5 mL). The mixture was sparged with N₂, [(prop-2-yn-1-yl)oxy]benzene (0.24 mL, 1.89 mmol, 1.2 eq) was added and the mixture was heated at 80° C. for 8 h. The reaction was partitioned between EtOAc and water, and the organic phase was washed with brine, dried (MgSO₄) and concentrated in vacuo. Purification by flash chromatography (10 g silica cartridge) eluting with a gradient of 0-5% EtOAc in heptane afforded Example 165A as an orange solid (292 mg, 1.19 mmol, 75%). ¹H NMR (400 MHz, DMSO-d₆) δ ppm: 7.48-7.41 (m, 2H), 7.38-7.28 (m, 3H), 7.28-7.19 (m, 2H), 7.06-7.02 (m, 2H), 6.99 (tt, J=7.3, 1.1 Hz, 1H), 5.05 (s, 2H), 3.57 (d, J=1.8 Hz, 2H).

### Example 165B 2″-(3-phenoxyprop-1-yn-1-yl)dispiro[[1,3]dioxolane-2,1′-cyclohexane-4′,1″-indene]

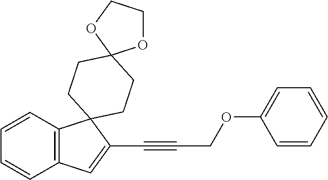 [SMILES: C(#CC1=Cc2ccccc2C12CCC1(CC2)OCCO1)COc1ccccc1]

Using General procedure 8a and Example 165A as the appropriate indene, Example 165B was isolated as a cream solid. LRMS calculated for C₂₅H₂₄O₃: 372; found: 373 (M+H).

### Example 165C 2′-(3-phenoxyprop-1-yn-1-yl)spiro[cyclohexane-1,1′-inden]-4-one

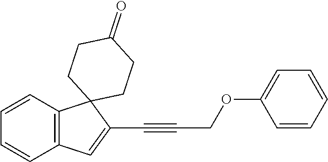 [SMILES: O=C1CCC2(CC1)C(C#CCOc1ccccc1)=Cc1ccccc12]

Using General procedure 9 and Example 165B as the appropriate ketal, Example 165C was isolated as a cream solid. ¹H NMR (400 MHz, DMSO-d₆) δ ppm: 7.74 (dd, J=7.4, 1.1 Hz, 1H), 7.46-7.39 (m, 1H), 7.36-7.21 (m, 4H), 7.16 (s, 1H), 7.07-6.91 (m, 3H), 5.11 (s, 2H), 2.74-2.57 (m, 4H), 2.05-1.92 (m, 2H), 1.92-1.82 (m, 2H).

### Example 165 (1r,4r)-4-(3-bromoanilino)-2′-(3-phenoxyprop-1-yn-1-yl)spiro[cyclohexane-1,1′-indene]-4-carboxylic acid

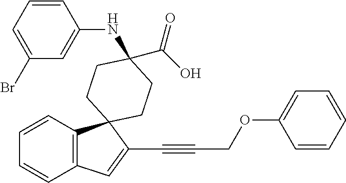 [SMILES: O=C(O)[C@]1(Nc2cccc(Br)c2)CC[C@@]2(CC1)C(C#CCOc1ccccc1)=Cc1ccccc12]

Using General procedure 10 and Example 165C as the appropriate ketone and 3-bromoaniline as the appropriate aniline, a single diastereoisomer, Example 165 was obtained as a cream foam. LRMS calculated for C₃₀H₂₆BrNO₃: 527; found: 528 (M+H). ¹H NMR (400 MHz, DMSO-d₆) δ ppm: 12.82 (br s, 1H), 7.49-7.43 (m, 1H), 7.39-7.24 (m, 5H), 7.12 (s, 1H), 7.08-6.97 (m, 4H), 6.87 (t, J=2.0 Hz, 1H), 6.74-6.66 (m, 1H), 6.68-6.63 (m, 1H), 6.37 (br s, 1H), 5.03 (s, 2H), 2.82-2.72 (m, 2H), 2.14-1.92 (m, 4H), 1.59-1.49 (m, 2H).

### Example 166

### Example 166A (1r,4r)-4-(3-chloroanilino)-2′-[(E)-2-ethoxyethenyl]spiro[cyclohexane-1,1′-indene]-4-carboxylic acid

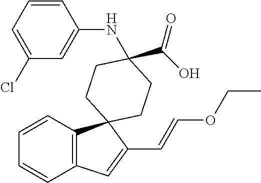 [SMILES: CCO/C=C/C1=Cc2ccccc2[C@]12CC[C@@](Nc1cccc(Cl)c1)(C(=O)O)CC2]

To a solution of Example 75 (450 mg, 1.04 mmol, 1 eq) in 1,4-dioxane (10 mL) and water (2 mL) was added 2-[(E)-2-ethoxyethenyl]-4,4,5,5-tetramethyl-1,3,2-dioxaborolane (0.26 mL, 1.25 mmol, 1.2 eq) and K₃PO₄ (0.88 g, 4.16 mmol, 4 eq). The mixture was sparged with N₂, Pd(dppf)Cl₂×DCM (43 mg, 0.05 mmol, 0.05 eq) was added and the mixture was heated at 110° C. for 1 h under microwave irradiation. The reaction was partitioned between DCM and brine, the aq. phase was extracted with DCM, and the combined organic extracts were washed with brine, dried (PTFE phase separator) and concentrated in vacuo. Purification by automated flash chromatography (CombiFlash Rf, 12 g RediSep™ silica cartridge) eluting with a gradient of 0-55% EtOAc in heptane afforded Example 166A as a yellow powder (315 mg, 0.74 mmol, 72%). LRMS calculated for C₂₅H₂₆ClNO₃: 423; found: 424 (M+H).

### Example 166B (1r,4r)-4-(3-chloroanilino)-2′-(2-oxoethyl)spiro[cyclohexane-1,1′-indene]-4-carboxylic acid

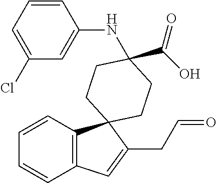 [SMILES: O=CCC1=Cc2ccccc2[C@]12CC[C@@](Nc1cccc(Cl)c1)(C(=O)O)CC2]

Using General procedure 9 and Example 166A, Example 166B was isolated as a yellow glass. LRMS calculated for C₂₃H₂₂ClNO₃: 395; found: 396 (M+H).

### Example 166 (1r,4r)-2′-(2-anilinoethyl)-4-(3-chloroanilino)spiro[cyclohexane-1,1′-indene]-4-carboxylic acid

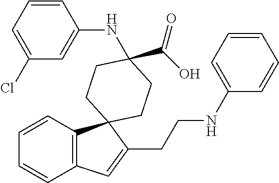 [SMILES: O=C(O)[C@]1(Nc2cccc(Cl)c2)CC[C@]2(CC1)C(CCNc1ccccc1)=Cc1ccccc12]

To a solution of Example 166B (100 mg, 0.25 mmol, 1 eq) in MeOH (1.5 mL), with 4\acute{Å} molecular sieves, was added aniline (28 μL, 0.3 mmol, 1.2 eq) followed by STAB (80 mg, 0.38 mmol, 1.5 eq) and the mixture was stirred at rt for 18 h. Further aniline (28 μL, 0.3 mmol, 1.2 eq) and STAB (80 mg, 0.38 mmol, 1.5 eq) were added and stirred for a further 18 h at rt. The mixture was partitioned between DCM and water, the organic phase was washed with 1 M aq. HCl solution, brine, dried (PTFE phase separator) and concentrated in vacuo. Purification by reverse phase automated flash chromatography (CombiFlash Rf, C18 4.3 g RediSep column) eluting with a gradient of 30-100% MeCN in water afforded Example 166 as a white powder (4 mg, 0.01 mmol, 3%). LRMS calculated for C₂₉H₂₉ClN₂O₂: 472; found: 473 (M+H). ¹H NMR (400 MHz, DMSO-d₆) δ ppm: 7.70 (d, J=7.5 Hz, 1H), 7.33-7.20 (m, 2H), 7.16-7.03 (m, 4H), 6.67-6.49 (m, 7H), 6.36 (br s, 1H), 5.71 (br s, 1H), 2.52-2.36 (m, 4H), 2.25-2.08 (m, 4H), 0.97-0.89 (m, 2H).

### Example 167 (1r,4r)-4-(3-chloroanilino)-2′-[3-(4-oxopyridin-1 (4H)-yl)propyl]-2′,3′-dihydrospiro[cyclohexane-1,1′-indene]-4-carboxylic acid

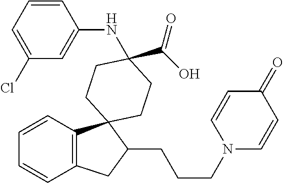

To a stirred solution of Preparation 9c (116 mg, 0.23 mmol, 1 eq.) in MeCN (5 mL) was added 4-hydroxypyridine (31 mg, 0.32 mmol, 1.4 eq) and K₂CO₃ (79 mg, 0.57 mmol, 2.5 eq). The mixture was heated at 75° C. for 18 h and then cooled to rt. The mixture was diluted with DCM and washed with water. The organic phase was dried (MgSO₄) and concentrated in vacuo.

Purification by automated flash chromatography (CombiFlash Rf, 4 g RediSep™ silica cartridge) eluting with a gradient of 0-10% MeOH in DCM afforded an intermediate which was hydrolysed according to General procedure 33b to obtain Example 167 as a racemate, isolated as an off-white powder (24 mg, 0.05 mmol, 21%). LRMS calculated for C₂₉H₃₁ClN₂O₃: 490; found: 491 (M+H). ¹H NMR 400 MHz, DMSO-d₆) δ ppm: 12.70 (br s, 1H), 7.72-7.62 (m, 2H), 7.37 (d, J=6.6 Hz, 1H), 7.24-7.11 (m, 3H), 7.08 (t, J=8.1 Hz, 1H), 6.62 (t, J=2.1 Hz, 1H), 6.55 (td, J=8.2, 2.0 Hz, 2H), 6.21 (br s, 1H), 6.09-6.03 (m, 2H), 3.86 (t, J=7.4 Hz, 2H), 2.99 (dd, J=15.6, 7.1 Hz, 1H), 2.46-2.33 (m, 1H), 2.16-2.06 (m, 1H), 2.05-1.93 (m, 2H), 1.92-1.76 (m, 3H), 1.74-1.62 (m, 2H), 1.52-1.32 (m, 4H), 1.19-1.07 (m, 1H).

### Example 168 (1r,4r)-2′-benzyl-4-(3-chloroanilino)spiro[cyclohexane-1,1′-indene]-4-carboxylic acid

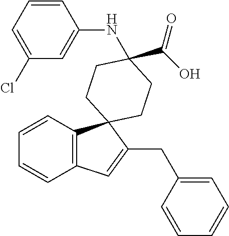 [SMILES: O=C(O)[C@]1(Nc2cccc(Cl)c2)CC[C@]2(CC1)C(Cc1ccccc1)=Cc1ccccc12]

Using General procedure 27a and Example 75 as the appropriate 2-bromo-indene derivative and benzylzinc bromide as the appropriate zinc reagent, Example 168 was obtained as an off-white powder. LRMS calculated for C₂₈H₂₆ClNO₂: 443; found: 444 (M+H). ¹H NMR (400 MHz, DMSO-d₆) δ ppm: 12.83 (br s, 1H), 7.68 (d, J=7.5 Hz, 1H), 7.35-7.18 (m, 7H), 7.16-7.07 (m, 2H), 6.69 (t, J=2.1 Hz, 1H), 6.61-6.56 (m, 2H), 6.37 (br s, 1H), 6.07-6.05 (m, 1H), 3.61-3.56 (m, 2H), 2.48-2.35 (m, 2H), 2.31-2.17 (m, 4H), 0.98-089 (m, 2H).

### Example 169 (1r,4r)-2′-benzyl-4-(3-chloroanilino)-2′,3′-dihydrospiro[cyclohexane-1,1′-indene]-4-carboxylic acid

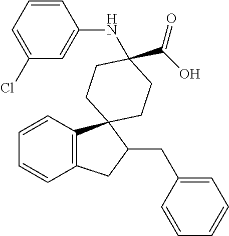

Using General procedure 19 and Example 168 as the appropriate indene afforded a racemate, Example 169, isolated as a colourless glass. LRMS calculated for C₂₈H₂₈ClNO₂: 445; found: 446 (M+H). ¹H NMR (400 MHz, DMSO-d₆) δ ppm: 12.76 (br s, 1H), 7.38 (d, J=7.2 Hz, 1H), 7.34-7.28 (m, 2H), 7.25-7.11 (m, 6H), 7.08 (t, J=8.1 Hz, 1H), 6.66 (t, J=2.1 Hz, 1H), 6.60-6.54 (m, 2H), 6.31 (br s, 1H), 2.95-2.85 (m, 1H), 2.70-2.62 (m, 1H), 2.58-2.40 (m, 2H), 2.33-2.15 (m, 3H), 2.06-1.93 (m, 3H), 1.93-1.82 (m, 1H), 1.60-1.49 (m, 2H).

### Example 170 (1s,4s)-4-(3-chloroanilino)-2′-[(pyridin-2-yl)ethynyl]spiro[cyclohexane-1,1′-indene]-4-carboxylic acid

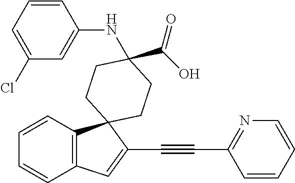 [SMILES: O=C(O)[C@]1(Nc2cccc(Cl)c2)CC[C@]2(CC1)C(C#Cc1ccccn1)=Cc1ccccc12]

To an oven-dried flask was added Example 76 (100 mg, 0.21 mmol, 1 eq), DIPA (0.15 mL, 1.04 mmol, 5 eq), CuI (2 mg, 0.01 mmol, 0.05 eq), PPh₃ (5 mg, 0.02 mmol, 0.1 eq) and Pd(PPh₃)₂Cl₂ (7 mg, 0.01 mmol, 0.05 eq) in toluene (3 mL). The mixture was sparged with N₂, 2-ethynylpyridine (0.02 mL, 0.21 mmol, 1 eq) was added and the mixture was heated at 80° C. for 48 h. The reaction was allowed to cool to rt, filtered through celite, washed with DCM and concentrated in vacuo. Purification using a pre-wetted (DCM) PE-AX cartridge (5 g), washed with DCM, MeOH and eluting in 5% HCOOH/DCM, followed by flash chromatography (4 g silica cartridge) eluting with a gradient of 0-5% MeOH in DCM afforded Example 170 as an off-white solid (14.4 mg, 0.03 mmol, 15%). LRMS calculated for C₂₈H₂₃ClN₂O₂: 454; found 455 (M+H). ¹H NMR (400 MHz, DMSO-d₆) δ ppm: 12.86 (br s, 1H), 8.64 (ddd, J=4.8, 1.8, 0.9 Hz, 1H), 7.88 (td, J=7.7, 1.8 Hz, 1H), 7.69 (dt, J=7.8, 1.1 Hz, 2H), 7.51-7.46 (m, 1H), 7.44 (ddd, J=7.6, 4.9, 1.2 Hz, 1H), 7.40-7.28 (m, 2H), 7.26 (s, 1H), 7.09 (t, J=8.1 Hz, 1H), 6.72 (t, J=2.1 Hz, 1H), 6.66-6.53 (m, 2H), 6.46 (br s, 1H), 2.47-2.27 (m, 6H), 1.23 (d, J=12.3 Hz, 2H).

### Example 171 (1r,4r)-4-(3-chloroanilino)-2′-(2-phenoxyethyl)spiro[cyclohexane-1,1′-indene]-4-carboxylic acid

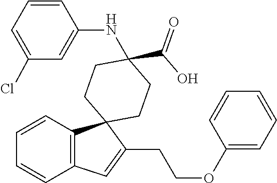 [SMILES: O=C(O)[C@]1(Nc2cccc(Cl)c2)CC[C@]2(CC1)C(CCOc1ccccc1)=Cc1ccccc12]

Using General procedure 30b and phenol as the appropriate alcohol and Preparation 8a as the appropriate indene, an intermediate was obtained which was hydrolysed according to General Procedure 33b to yield Example 171 as an off-white powder. LRMS calculated for C₂₉H₂₈ClNO₃: 473; found: 474 (M+H). ¹H NMR (400 MHz, DMSO-d₆) δ ppm: 12.83 (br s, 1H), 7.69 (d, J=7.5 Hz, 1H), 7.33-7.22 (m, 4H), 7.17-7.07 (m, 2H), 7.02-6.97 (m, 2H), 6.96-6.91 (m, 1H), 6.66 (t, J=2.1 Hz, 1H), 6.65-6.62 (m, 1H), 6.60-6.56 (m, 2H), 6.38 (br s, 1H), 4.28 (t, J=7.0 Hz, 2H), 2.70 (td, J=6.9, 1.6 Hz, 2H), 2.42 (td, J=14.3, 4.1 Hz, 2H), 2.26-2.12 (m, 4H), 0.99-0.90 (m, 2H).

### Example 172 (1s,4s)-4-(3-chloroanilino)-2′-(3-phenoxyprop-1-yn-1-yl)spiro[cyclohexane-1,1′-indene]-4-carboxylic acid

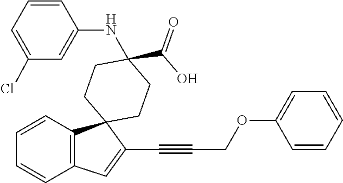 [SMILES: O=C(O)[C@]1(Nc2cccc(Cl)c2)CC[C@]2(CC1)C(C#CCOc1ccccc1)=Cc1ccccc12]

To an oven-dried flask was added Example 76 (100 mg, 0.21 mmol, 1 eq), DIPA (0.15 mL, 1.04 mmol, 5 eq), CuI (2 mg, 0.01 mmol, 0.05 eq), PPh₃ (5 mg, 0.02 mmol, 0.1 eq) and Pd(PPh₃)₂Cl₂ (7 mg, 0.01 mmol, 0.05 eq) in toluene (3 mL). The mixture was sparged with N₂, [(prop-2-yn-1-yl)oxy]benzene (27 μL, 0.21 mmol, 1 eq) was added and the mixture was heated at 80° C. for 48 h. The reaction was allowed to cool to rt, partitioned between DCM and water, and the organic phase was washed with brine, dried (PTFE phase separator) and concentrated in vacuo. Purification using a pre-wetted (DCM) PE-AX cartridge (5 g), washed with DCM, MeOH and eluting in 5% HCOOH/DCM, followed by flash chromatography (4 g silica cartridge) eluting with a gradient of 0-20% EtOAc in heptane afforded Example 172 as an off-white solid (9.1 mg, 0.02 mmol, 9%). LRMS calculated for C₃₀H₂₆ClNO₃: 483; found: 484 (M+H). ¹H NMR (400 MHz, DMSO-d₆) δ ppm:12.90 (br s, 1H), 7.70-7.56 (m, 1H), 7.47-7.35 (m, 1H), 7.35-7.23 (m, 2H), 7.23-7.13 (m, 2H), 7.13-7.03 (m, 2H), 7.03-6.96 (m, 2H), 6.91 (td, J=7.3, 1.1 Hz, 1H), 6.68 (t, J=2.1 Hz, 1H), 6.60 (dt, J=7.8, 2.7 Hz, 2H), 6.37 (br s, 1H), 5.08 (s, 2H), 2.45-2.10 (m, 6H), 1.12 (d, 2H).

### Example 173 (1s,4s)-4-(3-chloroanilino)-2′-[(pyridin-4-yl)ethynyl]spiro[cyclohexane-1,1′-indene]-4-carboxylic acid

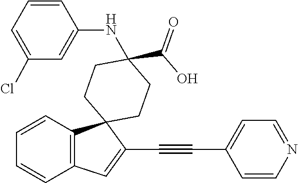 [SMILES: O=C(O)[C@]1(Nc2cccc(Cl)c2)CC[C@]2(CC1)C(C#Cc1ccncc1)=Cc1ccccc12]

To an oven-dried flask was added Example 76 (100 mg, 0.21 mmol, 1 eq), DIPA (0.15 mL, 1.04 mmol, 5 eq), CuI (2 mg, 0.01 mmol, 0.05 eq), PPh₃ (5 mg, 0.02 mmol, 0.1 eq) and Pd(PPh₃)₂Cl₂ (7 mg, 0.01 mmol, 0.05 eq) in toluene (3 mL). The mixture was sparged with N₂, 4-ethynylpyridine (22 mg, 0.21 mmol, 1 eq) was added and the mixture was heated at 80° C. for 48 h. The reaction was allowed to cool to rt, filtered through celite, washed with DCM and concentrated in vacuo. Purification using a pre-wetted (DCM) PE-AX cartridge (5 g), washed with DCM, MeOH and eluting in 5% HCOOH/DCM, followed by flash chromatography (4 g silica cartridge) eluting with a gradient of 0-5% MeOH in DCM afforded Example 173 as a cream powder (23.9 mg, 0.05 mmol, 25%). LRMS calculated for C₂₈H₂₃ClN₂O₂: 454; found: 455 (M+H). ¹H NMR (400 MHz, DMSO-d₆) δ ppm: 12.88 (br s, 1H), 8.69-8.62 (m, 2H), 7.70 (d, J=6.5 Hz, 1H), 7.62-7.44 (m, 3H), 7.40-7.29 (m, 2H), 7.27 (s, 1H), 7.11 (t, J=8.1 Hz, 1H), 6.73 (t, J=2.1 Hz, 1H), 6.61-6.55 (m, 2H), 6.53 (br s, 1H), 2.46-2.19 (m, 6H), 1.21 (d, 2H).

### Example 174

### Example 174A methyl (1r,4r)-4-(3-chloroanilino)-2′-{2-[(2-fluoropyridin-4-yl)oxy]ethyl}-2′,3′-dihydrospiro[cyclohexane-1,1′-indene]-4-carboxylate

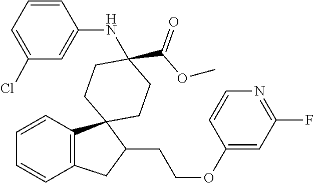

Using General procedure 40 and Preparation 8b as the appropriate mesylate and 2-fluoropyridin-4-ol as the appropriate alcohol afforded a racemate, Example 174A isolated as a white glass. LRMS calculated for C₂₉H₃₀ClFN₂O₃: 508; found: 509 (M+H).

### Example 174 (1r,4r)-4-(3-chloroanilino)-2′-{2-[(2-methoxypyridin-4-yl)oxy]ethyl}-2′,3′-dihydrospiro[cyclohexane-1,1′-indene]-4-carboxylic acid

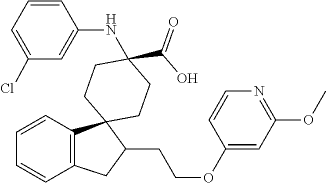

Using General procedure 33b and Example 174A as the appropriate ester afforded a racemate, Example 174, isolated as a white foam. LRMS calculated for C₂₉H₃₁ClN₂O₄: 506; found: 507 (M+H). ¹H NMR (400 MHz, DMSO-d₆) δ ppm: 12.72 (br s, 1H), 7.96 (d, J=5.9 Hz, 1H), 7.46-7.39 (m, 1H), 7.24-7.12 (m, 3H), 7.06 (t, J=8.1 Hz, 1H), 6.64-6.59 (m, 2H), 6.58-6.51 (m, 2H), 6.36 (d, J=2.2 Hz, 1H), 6.26 (br s, 1H), 4.21-4.07 (m, 2H), 3.82 (s, 3H), 3.01 (dd, J=15.7, 7.3 Hz, 1H), 2.64 (dd, J=15.8, 8.8 Hz, 1H), 2.49-2.36 (m, 1H), 2.21-1.90 (m, 6H), 1.80-1.58 (m, 2H), 1.56-1.45 (m, 1H), 1.40-1.29 (m, 1H).

### Example 175 and Example 176 Example 175A 2-[(dispiro[[1,3]dioxolane-2,1′-cyclohexane-4′,1″-inden]-2″-yl)ethynyl]pyridine

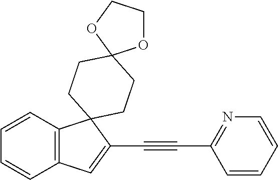 [SMILES: C(#Cc1ccccn1)C1=Cc2ccccc2C12CCC1(CC2)OCCO1]

To an oven-dried flask was added Example 76A (400 mg, 1.09 mmol, 1 eq), DIPA (0.37 mL, 2.63 mmol, 2.42 eq), CuI (4 mg, 0.02 mmol, 0.02 eq), PPh₃ (11 mg, 0.04 mmol, 0.04 eq) and Pd(PPh₃)₂Cl₂ (15 mg, 0.02 mmol, 0.02 eq) in toluene (5 mL). The mixture was sparged with N₂ before the addition of 2-ethynylpyridine (0.13 mL, 1.3 mmol, 1.2 eq) and then heated at 80° C. for 18 h. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-50% EtOAc in heptane afforded Example 175A as a yellow solid (169 mg, 0.49 mmol, 45%). LRMS calculated for C₂₃H₂₁NO₂: 343; found: 344 (M+H).

### Example 175B 2-[2-(2″,3″-dihydrodispiro[[1,3]dioxolane-2,1′-cyclohexane-4′,1″-inden]-2″-yl)ethyl]pyridine

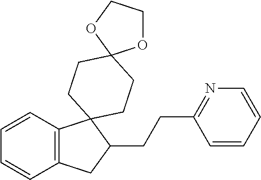 [SMILES: c1ccc(CCC2Cc3ccccc3C23CCC2(CC3)OCCO2)nc1]

To a solution of Example 175A (175 mg, 0.51 mmol, 1 eq) in EtOH (5 mL) was added 10% Pd/C (catalytic) under N₂. The mixture was evacuated and backfilled with N₂ (×3), then evacuated and backfilled with H₂ and shaken at rt for 18 h under an atmosphere of H₂. The reaction was filtered through celite, eluted with MeOH and the filtrate was concentrated under reduced pressure to afford a racemate, Example 175B as a yellow oil (178 mg, 0.51 mmol, 100%). LRMS calculated for C₂₃H₂₇NO₂: 349; found: 350 (M+H).

### Example 175C 2′-[2-(pyridin-2-yl)ethyl]-2′,3′-dihydrospiro[cyclohexane-1,1′-inden]-4-one

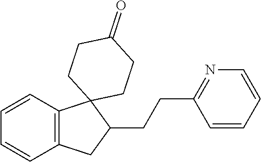 [SMILES: O=C1CCC2(CC1)c1ccccc1CC2CCc1ccccn1]

Using General procedure 9 and Example 175B as the appropriate ketal afforded a racemate, Example 175C, isolated as a yellow. LRMS calculated for C₂₁H₂₃NO: 305; found: 306 (M+H).

### Example 175 (1r,4r)-4-(3-bromoanilino)-2′-[2-(pyridin-2-yl)ethyl]-2′,3′-dihydrospiro[cyclohexane-1,1′-indene]-4-carboxylic acid

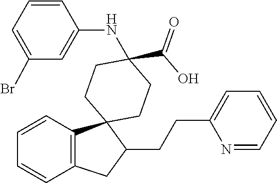

and

### Example 176 (1s,4s)-4-(3-bromoanilino)-2′-[2-(pyridin-2-yl)ethyl]-2′,3′-dihydrospiro[cyclohexane-1,1′-indene]-4-carboxylic acid

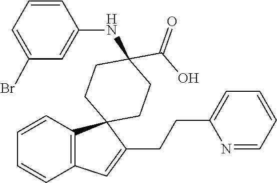 [SMILES: O=C(O)[C@]1(Nc2cccc(Br)c2)CC[C@@]2(CC1)C(CCc1ccccn1)=Cc1ccccc12]

Using General procedure 10 and Example 175C as the appropriate ketone and 3-bromoaniline as the appropriate aniline afforded a mixture of diastereoisomers. They were separated by automated flash column chromatography (CombiFlash Rf, 4 g RediSep™ silica cartridge) using a gradient of 0-10% MeOH in DCM. The racemic diastereoisomer eluting earlier was collected as Example 175. LRMS calculated for C₂₈H₂₉BrN₂O₂: 504; found: 505 (M+H). ¹H NMR (400 MHz, DMSO-d₆) δ ppm: 12.68 (br s, 1H), 8.47 (ddd, J=4.8, 1.9, 0.9 Hz, 1H), 7.69 (td, J=7.6, 1.9 Hz, 1H), 7.41-7.34 (m, 1H), 7.31-7.26 (m, 1H), 7.25-7.11 (m, 4H), 7.00 (t, J=8.1 Hz, 1H), 6.77 (t, J=2.1 Hz, 1H), 6.70-6.65 (m, 1H), 6.59-6.53 (m, 1H), 6.21 (br s, 1H), 3.02 (dd, J=15.6, 7.1 Hz, 1H), 2.94-2.84 (m, 1H), 2.78-2.59 (m, 2H), 2.46-2.35 (m, 1H), 2.15-2.04 (m, 1H), 2.04-1.82 (m, 5H), 1.81-1.68 (m, 1H), 1.61-1.48 (m, 1H), 1.47-1.32 (m, 2H).

The racemic diastereoisomer eluting later was collected as Example 176. LRMS calculated for C₂₈H₂₉BrN₂O₂: 504; found: 505 (M+H). ¹H NMR (400 MHz, DMSO-d₆) δ ppm:62 (br s, 1H), 8.46 (ddd, J=4.9, 1.9, 0.9 Hz, 1H), 7.68 (td, J=7.6, 1.8 Hz, 1H), 7.30-7.25 (dt, J=7.8, 1.1 Hz, 1H), 7.24-7.10 (m, 5H), 7.01 (t, J=8.1 Hz, 1H), 6.79 (t, J=2.1 Hz, 1H), 6.71-6.65 (m, 1H), 6.61-6.55 (m, 1H), 6.23 (br s, 1H), 3.07 (dd, J=16.0, 6.8 Hz, 1H), 2.92-2.81 (m, 1H), 2.78-2.66 (m, 2H), 2.30-2.22 (m, 1H), 2.10-1.82 (m, 6H), 1.62-1.45 (m, 3H), 1.36-1.23 (m, 1H).

### Example 177 (1r,4r)-4-(3-chloroanilino)-2′-(2-phenoxyethyl)-2′,3′-dihydrospiro[cyclohexane-1,1′-indene]-4-carboxylic acid

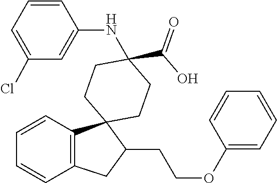

Using General procedure 40 and Preparation 8b as the appropriate mesylate and phenol as the appropriate alcohol afforded an intermediate which was hydrolysed according to General procedure 33b. Purification of the crude product by reverse phase automated flash chromatography (CombiFlash Rf, C18 4.3 g RediSep column) eluting with a gradient of 35-100% MeCN in water, followed by a pre-wetted (MeOH) PE-AX cartridge (2 g), washed with DCM, MeOH and eluting with 15% HCOOH/DCM afforded a racemate, Example 177 as a white powder. LRMS calculated for C₂₉H₃₀ClNO₃: 475; found: 476 (M+H). ¹H NMR (400 MHz, DMSO-d₆) δ ppm: 12.71 (br s, 1H), 7.43 (d, J=6.8 Hz, 1H), 7.32-7.25 (m, 2H), 7.24-7.14 (m, 3H), 7.07 (t, J=8.1 Hz, 1H), 7.00-6.88 (m, 3H), 6.63 (t, J=2.1 Hz, 1H), 6.55 (dd, J=8.1, 2.1 Hz, 2H), 6.23 (br s, 1H), 4.14-4.00 (m, 2H), 3.01 (dd, J=15.6, 7.2 Hz, 1H), 2.66 (dd, J=15.6, 8.5 Hz, 1H), 2.48-2.38 (m, 1H), 2.23-2.11 (m, 1H), 2.11-1.92 (m, 5H), 1.76 (t, J=12.3 Hz, 1H), 1.63 (tt, J=12.3, 5.9 Hz, 1H), 1.56-1.45 (m, 1H), 1.36 (d, J=13.4 Hz, 1H).

### Example 178 (1r,4r)-4-(3-chloroanilino)-2′-{2-[(thieno[3,2-b]pyridin-7-yl)oxy]ethyl}spiro[cyclohexane-1,1′-indene]-4-carboxylic acid

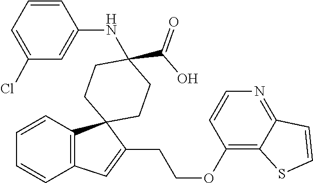 [SMILES: O=C(O)[C@]1(Nc2cccc(Cl)c2)CC[C@]2(CC1)C(CCOc1ccnc3ccsc13)=Cc1ccccc12]

Using General procedure 30b and thieno[3,2-b]pyridine-7-ol as the appropriate alcohol and Preparation 8a as the appropriate indene, an intermediate was obtained, which was hydrolysed according to General procedure 33b. Purification by automated flash column chromatography (CombiFlash Rf, 4 g RediSep™ silica cartridge) eluting with a gradient of 0-10% MeOH in DCM, followed by a pre-wetted (DCM) PE-AX cartridge (5 g), washed with DCM, MeOH and eluting with 5% HCOOH/DCM, afforded Example 178 as a white powder. LRMS calculated for C₃₀H₂₇ClN₂O₃S: 530; found: 531 (M+H). ¹H NMR (400 MHz, DMSO-d₆) δ ppm: 8.54 (d, J=5.4 Hz, 1H), 8.03 (d, J=5.4 Hz, 1H), 7.71 (d, J=7.6 Hz, 1H), 7.52 (d, J=5.4 Hz, 1H), 7.30 (dd, J=7.4, 1.3 Hz, 1H), 7.24 (t, J=7.4 Hz, 1H), 7.18-7.06 (m, 3H), 6.70-6.66 (m, 2H), 6.61-6.54 (m, 2H), 6.36 (br s, 1H), 4.60 (t, J=6.8 Hz, 2H), 2.85-2.78 (m, 2H), 2.48-2.37 (m, 2H), 2.27-2.16 (m, 4H), 1.00-0.92 (m, 2H).

### Example 179 (1s,4s)-2′-[(benzyloxy)methyl]-4-(3-chloroanilino)spiro[cyclohexane-1,1′-indene]-4-carboxylic acid

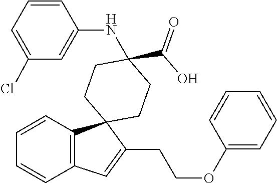 [SMILES: O=C(O)[C@]1(Nc2cccc(Cl)c2)CC[C@]2(CC1)C(CCOc1ccccc1)=Cc1ccccc12]

Using General procedure 20 and BnOH as the appropriate alcohol, Example 179 was obtained. ¹H NMR (500 MHz, DMSO-d₆) δ ppm: 7.70 (d, 1H), 7.39-7.20 (m, 5H), 7.37-7.11 (m, 3H), 7.05 (t, 1H), 6.70 (br s, 1H), 6.65 (t, 1H), 6.55 (dm, 1H), 6.52 (dm, 1H), 6.24 (m, 1H), 4.56 (s, 2H), 4.34 (d, 2H), 2.40/2.14 (tm+dm, 4H), 2.18/0.99 (tm+dm, 4H). HRMS calculated for C₂₉H₂₈NO₃Cl: 473.1758; found: 474.1831 (M+H).

### Example 180

### Example 180A methyl (1s,4s)-2′-(benzylcarbamoyl)-4-(3-chloroanilino)spiro[cyclohexane-1,1′-indene]-4-carboxylate

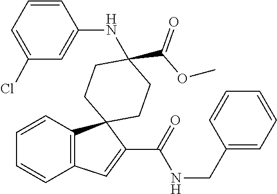 [SMILES: COC(=O)[C@]1(Nc2cccc(Cl)c2)CC[C@]2(CC1)C(C(=O)NCc1ccccc1)=Cc1ccccc12]

Using General procedure 21 and Preparation 7a as the appropriate carboxylic acid and 1-phenylmethanamine as the appropriate amine, Example 180A was obtained. ¹H NMR (500 MHz, DMSO-d₆) δ ppm: 8.75 (t, 1H), 7.78-7.19 (m, 9H), 7.47 (s, 1H), 7.08 (t, 1H), 6.61 (t, 1H), 6.58 (dm, 1H), 6.45 (dm, 1H), 6.32 (s, 1H), 4.40 (d, 2H), 3.68 (s, 3H), 2.88/0.98 (m+m, 4H), 2.37/2.27 (m+m, 4H). HRMS calculated for C₃₀H₂₉ClN₂O₃: 500.1867; found: 501.1951 (M+H).

### Example 180 (1s,4s)-2′-(benzylcarbamoyl)-4-(3-chloroanilino)spiro[cyclohexane-1,1′-indene]-4-carboxylic acid

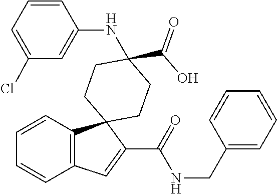 [SMILES: O=C(NCc1ccccc1)C1=Cc2ccccc2[C@]12CC[C@@](Nc1cccc(Cl)c1)(C(=O)O)CC2]

Using General procedure 33a and Example 180A as the appropriate ester, Example 180 was obtained. ¹H NMR (500 MHz, DMSO-d₆) δ ppm: 8.73 (t, 1H), 7.77 (d, 1H), 7.50 (dm, 1H), 7.46 (s, 1H), 7.37-7.18 (m, 7H), 7.05 (t, 1H), 6.62 (m, 1H), 6.55 (dm, 1H), 6.55 (dm, 1H), 6.13 (br, 1H), 4.41 (d, 2H), 2.85/0.96 (td+d, 4H), 2.37/2.17 (td+d, 4H). HRMS calculated for C₂₉H₂₇N₂O₃Cl: 486.1710; found: 487.1779 (M+H).

### Example 181

### Example 181A methyl (1s,4s)-2′-[benzyl(methyl)carbamoyl]-4-(3-chloroanilino)spiro[cyclohexane-1,1′-indene]-4-carboxylate

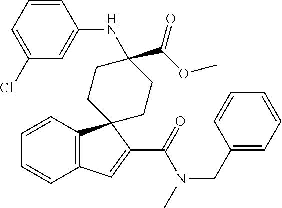 [SMILES: COC(=O)[C@]1(Nc2cccc(Cl)c2)CC[C@]2(CC1)C(C(=O)N(C)Cc1ccccc1)=Cc1ccccc12]

Using General procedure 21 and Preparation 7a as the appropriate carboxylic acid and N-methyl-1-phenyl-methanamine as the appropriate amine, Example 181A was obtained. ¹H NMR (500 MHz, DMSO-d₆) δ ppm: 7.72-7.16 (m, 9H), 7.10 (t, 1H), 6.84 (br s, 1H), 6.63 (t, 1H), 6.61 (dm, 1H), 6.47 (dm, 1H), 6.47 (br s, 1H), 4.64 (br s, 2H), 3.68 (s, 3H), 2.84 (s, 3H), 2.41/1.28 (m+m, 4H), 2.41/2.21 (m+m, 4H). HRMS calculated for C₃₁H₃₁ClN₂O₃: 514.2023; found 515.2101 (M+H).

### Example 181 (1s,4s)-2′-[benzyl(methyl)carbamoyl]-4-(3-chloroanilino)spiro[cyclohexane-1,1′-indene]-4-carboxylic acid

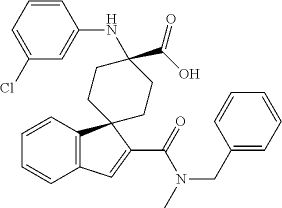 [SMILES: CN(Cc1ccccc1)C(=O)C1=Cc2ccccc2[C@]12CC[C@@](Nc1cccc(Cl)c1)(C(=O)O)CC2]

Using General procedure 33a and Example 181A as the appropriate ester, Example 181 was obtained. ¹H NMR (500 MHz, DMSO-d₆) δ ppm: 12.79 (br s, 1H), 7.74-7.16 (m, 9H), 7.10 (t, 1H), 6.82 (br s, 1H), 6.64 (t, 1H), 6.58 (dm, 1H), 6.55 (dm, 1H), 6.36 (br s, 1H), 4.64 (s, 2H), 2.84 (s, 3H), 2.40/1.27 (m+m, 4H), 2.40/2.18 (m+m, 4H). HRMS calculated for C₃₀H₂₉ClN₂O₃: 500.1867; found 501.1939 (M+H).

### Example 182 (1r,4r)-4-(3-chloroanilino)-2′-[3-(5-methyl-6-oxopyrimidin-1 (6H)-yl)propyl]spiro[cyclohexane-1,1′-indene]-4-carboxylic acid

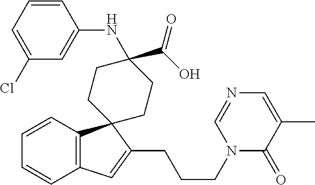 [SMILES: Cc1cncn(CCCC2=Cc3ccccc3[C@]23CC[C@@](Nc2cccc(Cl)c2)(C(=O)O)CC3)c1=O]

Using General procedure 30b and Preparation 9a as the appropriate indene and 5-methylpyrimidin-4-ol as the appropriate alcohol, an intermediate was obtained, which was hydrolysed according to General procedure 33b to obtain Example 182 as a white powder. LRMS calculated for C₂₉H₃₀ClN₃O₃: 503; found: 504 (M+H). ¹H NMR (400 MHz, DMSO-d₆) δ ppm: 12.80 (br s, 1H), 8.37 (s, 1H), 7.83-7.80 (m, 1H), 7.66 (d, J=7.6 Hz, 1H), 7.29 (dd, J=7.5, 1.3 Hz, 1H), 7.23 (td, J=7.4, 0.9 Hz, 1H), 7.16-7.08 (m, 2H), 6.64 (t, J=2.1 Hz, 1H), 6.61-6.54 (m, 3H), 6.36 (br s, 1H), 4.03-3.96 (m, 2H), 2.46-2.35 (m, 2H), 2.29-1.99 (m, 8H), 1.95 (d, J=1.1 Hz, 3H), 0.94-0.86 (m, 2H).

### Example 183 (1r,4r)-4-(3-chloroanilino)-2′-[3-(5-methyl-6-oxopyrimidin-1 (6H)-yl)propyl]-2′,3′-dihydrospiro[cyclohexane-1,1′-indene]-4-carboxylic acid

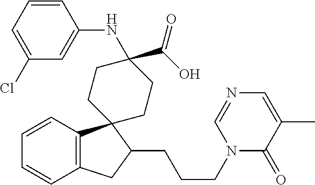

Using General procedure 40 and Preparation 9c as the appropriate mesylate and 5-methylpyrimidin-4-ol as the appropriate alcohol, an intermediate was obtained, which was hydrolysed according to General procedure 33b to obtain Example 183 as a racemate, isolated as a white powder. LRMS calculated for C₂₉H₃₂ClN₃O₃: 505; found: 506 (M+H). ¹H NMR (400 MHz, DMSO-d₆) δ ppm: 8.36 (s, 1H), 7.83-7.80 (m, 1H), 7.41-7.32 (m, 1H), 7.23-7.11 (m, 3H), 7.06 (t, J=8.1 Hz, 1H), 6.62 (t, J=2.1 Hz, 1H), 6.58-6.52 (m, 2H), 6.22 (br s, 1H), 3.98-3.83 (m, 2H), 2.99 (dd, J=15.7, 7.3 Hz, 1H), 2.58-2.49 (m, 1H), 2.46-2.35 (m, 1H), 2.17-2.07 (m, 1H), 2.06-1.34 (m, 13H), 1.24-1.11 (m, 1H).

### Example 184

### Example 184A methyl (1s,4s)-2′-{[benzyl(methyl)amino]methyl}-4-(3-chloroanilino)spiro[cyclohexane-1,1′-indene]-4-carboxylate

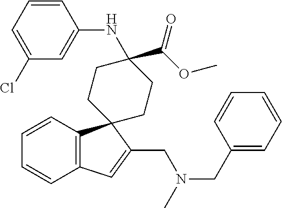 [SMILES: COC(=O)[C@]1(Nc2cccc(Cl)c2)CC[C@]2(CC1)C(CN(C)Cc1ccccc1)=Cc1ccccc12]

Example 181A (50 mg, 0.1 mmol) was dissolved in dry THE (1 mL) and the flask was flushed with N₂. BH₃×THF in THE (1 M, 300 μL, 0.3 mmol) was added to the mixture and stirred at rt, then at 70° C., but no conversion could be observed. BH₃×SMe₂ in THF (2 M, 33 μL, 0.06 mmol) was added to the mixture and stirred at rt overnight. The reaction was quenched by the addition of MeOH and water. The mixture was concentrated under reduced pressure and purified via flash chromatography using heptane and EtOAc as eluents to give Example 184A. LRMS calculated for C₃₁H₃₃ClN₂O₂: 500.2; found 499.2 (M−H).

### Example 184 (1s,4s)-2′-{[benzyl(methyl)amino]methyl}-4-(3-chloroanilino)spiro[cyclohexane-1,1′-indene]-4-carboxylic acid

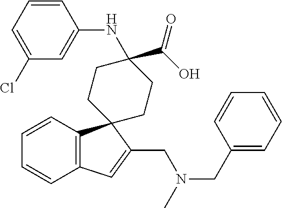 [SMILES: CN(Cc1ccccc1)CC1=Cc2ccccc2[C@]12CC[C@@](Nc1cccc(Cl)c1)(C(=O)O)CC2]

Using General procedure 33a and Example 184A as the appropriate ester, Example 184 was obtained. ¹H NMR (500 MHz, DMSO-d₆) δ ppm: 7.97 (d, 1H), 7.33 (m, 3H), 7.25-7.18 (m, 2H), 7.25 (m, 2H), 7.13 (t, 1H), 7.09 (t, 1H), 6.72 (s, 1H), 6.67 (t, 1H), 6.58 (dm, 1H), 6.57 (dm, 1H), 6.32 (br, 1H), 3.53 (s, 2H), 3.25 (s, 2H), 2.40/2.18 (td+d, 4H), 2.29/0.93 (td+d, 4H), 2.11 (s, 3H). HRMS calculated for C₃₀H₃₁ClN₂O₂: 486.2074; found 487.2146 (M+H).

### Example 185 (1r,4r)-4-(3-chloroanilino)-2′-{2-[(1H-indol-4-yl)oxy]ethyl}-2′,3′-dihydrospiro[cyclohexane-1,1′-indene]-4-carboxylic acid

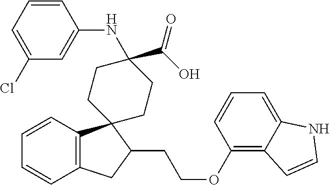

Using General procedure 40 and Preparation 8b as the appropriate mesylate and 4-hydroxyindole as the appropriate alcohol, an intermediate was obtained, which was hydrolysed according to General procedure 33b to obtain Example 185, isolated as a white powder. LRMS calculated for C₃₁H₃₁ClN₂O₃: 514; found: 515 (M+H). ¹H NMR (400 MHz, DMSO-d₆) δ ppm: 12.71 (br s, 1H), 11.05 (s, 1H), 7.45-7.40 (m, 1H), 7.24-7.13 (m, 4H), 7.07 (t, J=8.1 Hz, 1H), 7.00-6.95 (m, 2H), 6.65 (t, J=2.2 Hz, 1H), 6.60-6.54 (m, 2H), 6.53-6.48 (m, 1H), 6.44-6.41 (m, 1H), 6.29 (br s, 1H), 4.25-4.11 (m, 2H), 3.05 (dd, J=15.7, 7.2 Hz, 1H), 2.75-2.67 (m, 1H), 2.33-2.23 (m, 1H), 2.18-1.91 (m, 5H), 1.85-1.75 (m, 1H), 1.74-1.62 (m, 1H), 1.59-1.48 (m, 1H), 1.46-1.36 (m, 1H).

### Example 186 (1r,4r)-4-(3-chloroanilino)-2′-{2-[(thieno[3,2-b]pyridin-7-yl)oxy]ethyl}-2′,3′-dihydrospiro[cyclohexane-1,1′-indene]-4-carboxylic acid

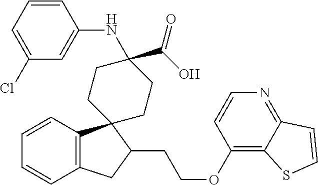

Using General procedure 40 and Preparation 8b as the appropriate mesylate and thieno[3,2-b]pyridine-7-ol as the appropriate alcohol, an intermediate was obtained, which was hydrolysed according to General procedure 33b to obtain Example 186, isolated as a white powder. LRMS calculated for C₃₀H₂₉ClN₂O₃S: 532; found: 533 (M+H). ¹H NMR (400 MHz, DMSO-d₆) δ ppm: 12.71 (br s, 1H), 8.58 (d, J=5.5 Hz, 1H), 8.08 (d, J=5.4 Hz, 1H), 7.54 (d, J=5.3 Hz, 1H), 7.46-7.41 (m, 1H), 7.24-7.13 (m, 3H), 7.11-7.04 (m, 2H), 6.64 (t, J=2.1 Hz, 1H), 6.58-6.52 (m, 2H), 6.26 (br s, 1H), 4.50-4.39 (m, 2H), 3.06 (dd, J=15.6, 7.2 Hz, 1H), 2.75-2.68 (m, 1H), 2.28-2.17 (m, 1H), 2.17-1.91 (m, 5H), 1.83-1.70 (m, 2H), 1.57-1.47 (m, 1H), 1.43-1.34 (m, 1H).

### Example 187 (1r,4r)-4-(3-chloroanilino)-2′-{2-[(2-fluoropyridin-4-yl)oxy]ethyl}-2′,3′-dihydrospiro[cyclohexane-1,1′-indene]-4-carboxylic acid

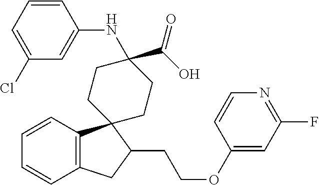

Using General procedure 33a and Example 174A as the appropriate ester afforded a racemate, Example 187 isolated as a white powder. LRMS calculated for C₂₈H₂₈ClFN₂O₃: 494; found: 495 (M+H). ¹H NMR (400 MHz, DMSO-d₆) δ ppm: 12.75 (br s, 1H), 8.04 (d, J=5.9 Hz, 1H), 7.46-7.40 (m, 1H), 7.24-7.13 (m, 3H), 7.05 (t, J=8.1 Hz, 1H), 6.95 (ddd, J=5.8, 2.1, 1.2 Hz, 1H), 6.79 (d, J=2.1 Hz, 1H), 6.63 (t, J=2.1 Hz, 1H), 6.57-6.51 (m, 2H), 6.23 (br s, 1H), 4.29-4.15 (m, 2H), 3.01 (dd, J=15.7, 7.2 Hz, 1H), 2.69-2.61 (m, 1H), 2.49-2.38 (m, 1H), 2.19-1.88 (m, 6H), 1.79-1.60 (m, 2H), 1.56-1.46 (m, 1H), 1.39-1.31 (m, 1H).

### Example 188 (1r,4r)-4-(3-chloroanilino)-2′-{2-[(3-fluoropyridin-4-yl)oxy]ethyl}-2′,3′-dihydrospiro[cyclohexane-1,1′-indene]-4-carboxylic acid

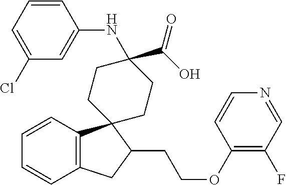

Using General procedure 40 and Preparation 8b as the appropriate mesylate and 3-fluoro-4-hydroxypyridine as the appropriate alcohol, an intermediate was obtained, which was hydrolysed according to General procedure 33a to obtain Example 188, isolated as a white powder. LRMS calculated for C₂₈H₂₈ClFN₂O₃: 494; found: 495 (M+H). ¹H NMR (400 MHz, DMSO-d₆) δ ppm: 8.42 (d, J=3.3 Hz, 1H), 8.29 (d, J=5.6 Hz, 1H), 7.46-7.42 (m, 1H), 7.30 (dd, J=7.6, 5.6 Hz, 1H), 7.23-7.12 (m, 3H), 7.03 (t, J=8.0 Hz, 1H), 6.64 (t, J=2.1 Hz, 1H), 6.58-6.48 (m, 2H), 6.17 (br s, 1H), 4.35-4.21 (m, 2H), 3.02 (dd, J=15.7, 7.2 Hz, 1H), 2.71-2.62 (m, 1H), 2.49-2.37 (m, 1H), 2.22-1.84 (m, 6H), 1.79-1.64 (m, 2H), 1.55-1.45 (m, 1H), 1.41-1.31 (m, 1H).

### Example 189 (1r,4r)-4-(3-chloroanilino)-2′-{2-[(3-methylpyridin-4-yl)oxy]ethyl}spiro[cyclohexane-1,1′-indene]-4-carboxylic acid

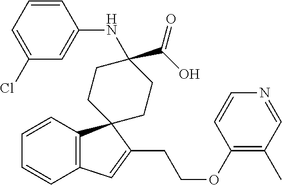 [SMILES: Cc1cnccc1OCCC1=Cc2ccccc2[C@]12CC[C@@](Nc1cccc(Cl)c1)(C(=O)O)CC2]

Using General procedure 31a and Preparation 8a as the appropriate alcohol and 4-chloro-3-methylpyridine as the appropriate aryl chloride, an intermediate was obtained, which was hydrolysed according to General procedure 33b to obtain Example 189, isolated as a white powder. LRMS calculated for C₂₉H₂₉ClN₂O₃: 488; found: 489 (M+H). ¹H NMR (400 MHz, DMSO-d₆) δ ppm: 8.29 (d, J=5.6 Hz, 1H), 8.21 (s, 1H), 7.82 (d, J=7.6 Hz, 1H), 7.27 (dd, J=7.5, 1.3 Hz, 1H), 7.20 (t, J=7.4 Hz, 1H), 7.13-7.04 (m, 2H), 6.93 (t, J=7.9 Hz, 1H), 6.68-6.56 (m, 3H), 6.39-6.33 (m, 1H), 5.83 (br s, 1H), 4.37 (t, J=6.8 Hz, 2H), 2.77-2.70 (m, 2H), 2.47-2.35 (m, 2H), 2.15-1.97 (m, 7H), 0.87-0.77 (m, 2H).

### Example 190 (1r,4r)-4-(3-chloroanilino)-2′-{2-[(3-fluoropyridin-4-yl)oxy]ethyl}spiro[cyclohexane-1,1′-indene]-4-carboxylic acid

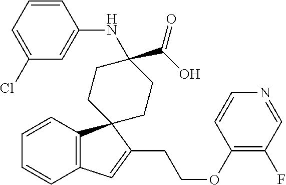 [SMILES: O=C(O)[C@]1(Nc2cccc(Cl)c2)CC[C@]2(CC1)C(CCOc1ccncc1F)=Cc1ccccc12]

Using General procedure 31a and Preparation 8a as the appropriate alcohol and 4-chloro-3-fluoropyridine as the appropriate aryl chloride, an intermediate was obtained, which was hydrolysed according to General procedure 33b to obtain Example 190, isolated as a white powder. LRMS calculated for C₂₈H₂₆ClFN₂O₃: 492; found: 493 (M+H). ¹H NMR (400 MHz, DMSO-d₆) δ ppm: 12.85 (br s, 1H), 8.44 (d, J=3.4 Hz, 1H), 8.30 (d, J=5.5 Hz, 1H), 7.73-7.68 (m, 1H), 7.37 (dd, J=7.6, 5.6 Hz, 1H), 7.33-7.29 (m, 1H), 7.25 (t, J=7.4 Hz, 1H), 7.18-7.12 (m, 1H), 7.11-7.05 (m, 1H), 6.68-6.52 (m, 4H), 6.33 (br s, 1H), 4.48 (t, J=7.0 Hz, 2H), 2.79-2.73 (m, 2H), 2.47-2.36 (m, 2H), 2.24-2.11 (m, 4H), 0.98-0.88 (m, 2H).

### Example 191

### Example 191A methyl (1r,4r)-4-(3-chloroanilino)-2′-{2-[(methanesulfonyl)oxy]ethyl}spiro[cyclohexane-1,1′-indene]-4-carboxylate

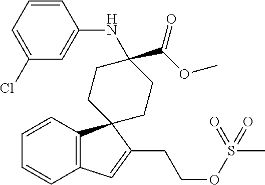 [SMILES: COC(=O)[C@]1(Nc2cccc(Cl)c2)CC[C@]2(CC1)C(CCOS(C)(=O)=O)=Cc1ccccc12]

To a solution of Preparation 8a (1 eq) in DCM (10 mL/mmol) at 0° C. was added TEA (2 eq) followed by the dropwise addition of MsCl (2 eq) and the mixture was stirred at rt until no further conversion was observed. Then it was partitioned between DCM and water, and the organic phase was washed with with sat. aq. NaHCO₃ solution, brine, dried (MgSO₄) and concentrated in vacuo to obtain Example 191A, isolated as a yellow oil. LRMS calculated for C₂₅H₂₈ClNO₅S: 489; found: 490 (M+H).

### Example 191 (1r,4r)-4-(3-chloroanilino)-2′-{2-[(2-fluoropyridin-4-yl)oxy]ethyl}spiro[cyclohexane-1,1′-indene]-4-carboxylic acid

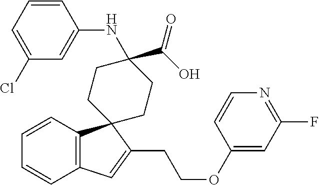 [SMILES: O=C(O)[C@]1(Nc2cccc(Cl)c2)CC[C@]2(CC1)C(CCOc1ccnc(F)c1)=Cc1ccccc12]

Using General procedure 40 and Example 191A as the appropriate mesylate and 2-fluoro-4-hydroxypyridine as the appropriate alcohol, an intermediate was obtained, which was hydrolysed according to General procedure 33b to obtain Example 191, isolated as a white powder. LRMS calculated for C₂₈H₂₆ClFN₂O₃: 492; found: 493 (M+H). ¹H NMR (400 MHz, DMSO-d₆) δ ppm: 12.84 (br s, 1H), 8.05 (d, J=5.8 Hz, 1H), 7.69 (d, J=7.5 Hz, 1H), 7.31 (dd, J=7.4, 1.3 Hz, 1H), 7.25 (td, J=7.4, 0.9 Hz, 1H), 7.15 (td, J=7.5, 1.4 Hz, 1H), 7.10 (t, J=8.1 Hz, 1H), 6.99 (ddd, J=5.9, 2.1, 1.2 Hz, 1H), 6.85 (d, J=2.1 Hz, 1H), 6.68-6.62 (m, 2H), 6.65-6.54 (m, 2H), 6.37 (br s, 1H), 4.43 (t, J=6.9 Hz, 2H), 2.76-2.68 (m, 2H), 2.48-2.36 (m, 2H), 2.25-2.11 (m, 4H), 0.94 (d, J=13.3 Hz, 2H).

### Example 192 (1r,4r)-4-(3-chloroanilino)-2′-{2-[(1H-indol-4-yl)oxy]ethyl}spiro[cyclohexane-1,1′-indene]-4-carboxylic acid

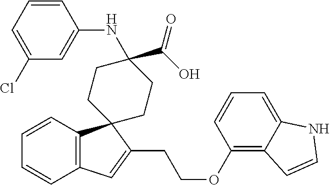 [SMILES: O=C(O)[C@]1(Nc2cccc(Cl)c2)CC[C@]2(CC1)C(CCOc1cccc3[nH]ccc13)=Cc1ccccc12]

Using General procedure 40 and Example 191A as the appropriate mesylate and 4-hydroxyindole as the appropriate alcohol, an intermediate was obtained, which was hydrolysed according to General procedure 33b to obtain Example 192, isolated as a white powder. LRMS calculated for C₃₁H₂₉ClN₂O₃: 512.19; found: 513.20 (M+H). ¹H NMR (400 MHz, DMSO-d₆) δ ppm: 12.84 (br s, 1H), 11.06 (s, 1H), 7.70 (d, J=7.6 Hz, 1H), 7.32-7.27 (m, 1H), 7.27-7.21 (m, 1H), 7.20-7.18 (m, 1H), 7.17-7.06 (m, 2H), 7.02-6.95 (m, 2H), 6.70-6.66 (m, 2H), 6.62-6.54 (m, 3H), 6.46-6.32 (m, 2H), 4.38 (t, J=6.9 Hz, 2H), 2.81-2.74 (m, 2H), 2.49-2.37 (m, 2H), 2.30-2.16 (m, 4H), 1.01-0.92 (m, 2H).

### Example 193

### Example 193A 3-[(3-methylpyridin-4-yl)oxy]propan-1-ol

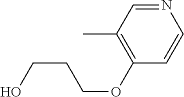

To a suspension of 4-bromo-3-methylpyridine×HCl (519 mg, 2.49 mmol, 1 eq) and 1,3-propanediol (0.90 mL, 12.4 mmol, 5 eq) in DMSO (4 mL) was added powdered NaOH (438 mg, 10.95 mmol, 4.4 eq) and the mixture was heated at 100° C. for 18 h. After cooling, the mixture was concentrated, and the residue was diluted with water. The mixture was extracted with DCM and the combined organic extracts were dried (MgSO₄) and concentrated under reduced pressure. Purification by automated reverse phase flash chromatography (CombiFlash Rf, C18 43 g RediSep column) eluting with a gradient of 10-80% MeCN in water afforded Example 193A as a white solid (94 mg, 0.56 mmol, 23%). LRMS calculated for C₉H₁₃NO₂: 167; found: 168 (M+H).

### Example 193B methyl (1s,4s)-4-(3-bromoanilino)-2′-iodospiro[cyclohexane-1,1′-indene]-4-carboxylate

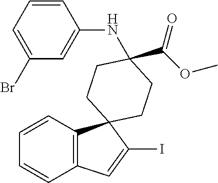 [SMILES: COC(=O)[C@]1(Nc2cccc(Br)c2)CC[C@]2(CC1)C(I)=Cc1ccccc12]

Using General procedure 17a and Example 77 as the appropriate amino acid, Example 193B was obtained. LRMS calculated for C₂₂H₂₁BrINO₂: 537; found 538 (M+H). ¹H NMR (400 MHz, DMSO-d₆) δ ppm: 7.75 (d, J=7.5 Hz, 1H), 7.36 (dd, J=7.4, 1.3 Hz, 1H), 7.32-7.27 (m, 1H), 7.24 (s, 1H), 7.20 (td, J=7.5, 1.4 Hz, 1H), 7.05 (t, J=8.0 Hz, 1H), 6.80 (t, J=2.1 Hz, 1H), 6.76-6.71 (m, 1H), 6.55-6.48 (m, 2H), 3.70 (s, 3H), 2.44 (td, J=14.2, 3.9 Hz, 2H), 2.33-2.24 (m, 2H), 2.13 (td, J=13.8, 3.9 Hz, 2H), 0.94-0.86 (m, 2H).

### Example 193 (1s,4s)-4-(3-bromoanilino)-2′-{3-[(3-methylpyridin-4-yl)oxy]propoxy}spiro[cyclohexane-1,1′-indene]-4-carboxylic acid

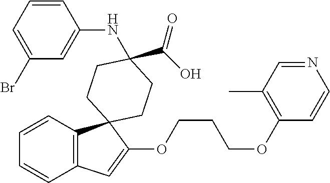 [SMILES: Cc1cnccc1OCCCOC1=Cc2ccccc2[C@]12CC[C@@](Nc1cccc(Br)c1)(C(=O)O)CC2]

To an oven-dried vial was added Example 193B (67 mg, 0.12 mmol, 1 eq), Example 193A (25 mg, 0.15 mmol, 1.2 eq), Cs₂CO₃ (122 mg, 0.37 mmol, 3 eq), allylpalladium(II) chloride dimer (2 mg, 0.01 mmol, 0.05 eq) and Josiphos SL-J009 (7 mg, 0.01 mmol, 0.1 eq) under N₂. Dry toluene (1 mL) was added and the mixture was sparged with N₂ and heated at 90° C. for 18 h under a N₂ atmosphere. The reaction was cooled, diluted with DCM, filtered and concentrated in vacuo. Purification by automated flash chromatography (CombiFlash Rf, 12 g RediSep™ silica cartridge) eluting with a gradient of 0-80% EtOAc in heptane afforded an intermediate, which was hydrolysed according to General procedure 33b to obtain Example 193 as a white solid (7.7 mg, 0.01 mmol, 11%). LRMS calculated for C₃₀H₃₁BrN₂O₄: 562; found: 563 (M+H). ¹H NMR (400 MHz, DMSO-d₆) δ ppm: 8.19 (s, 1H), 8.16 (d, J=5.6 Hz, 1H), 7.39 (d, J=7.4 Hz, 1H), 7.17-7.10 (m, 2H), 7.02-6.96 (m, 2H), 6.87 (d, J=5.6 Hz, 1H), 6.79 (t, J=2.1 Hz, 1H), 6.71-6.66 (m, 1H), 6.61-6.57 (m, 1H), 5.70 (s, 1H), 4.24 (t, J=6.2 Hz, 2H), 4.15 (t, J=6.2 Hz, 2H), 2.37-2.27 (m, 2H), 2.27-2.07 (m, 7H), 2.04-1.93 (m, 2H), 1.30-1.17 (m, 2H).

### Example 195

### Example 195A 7-[(but-3-yn-1-yl)oxy]thieno[3,2-b]pyridine

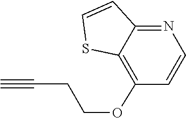

Using General procedure 30b and thieno[3,2-b]pyridine-7-ol instead of the appropriate indene and 3-butyn-1-ol as the appropriate alcohol, Example 195A was obtained as a cream powder. LRMS calculated for C_{II}H9NOS: 203; found: 204 (M+H).

### Example 195 (1s,4s)-4-(3-chloroanilino)-2′-{4-[(thieno[3,2-b]pyridin-7-yl)oxy]but-1-yn-1-yl}spiro[cyclohexane-1,1′-indene]-4-carboxylic acid

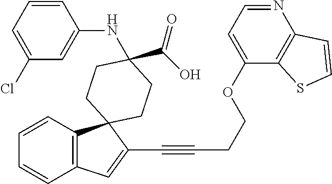 [SMILES: O=C(O)[C@]1(Nc2cccc(Cl)c2)CC[C@]2(CC1)C(C#CCCOc1ccnc3ccsc13)=Cc1ccccc12]

To an oven-dried flask was added Example 76 (50 mg, 0.1 mmol, 1 eq), DIPA (73 μL, 0.52 mmol, 5 eq), CuI (1 mg, 0.01 mmol, 0.05 eq), PPh₃ (3 mg, 0.01 mmol, 0.1 eq) and Pd(PPh₃)₂Cl₂ (4 mg, 0.01 mmol, 0.05 eq) in toluene (1 mL). The mixture was sparged with N₂ and then Example 195A (42.4 mg, 0.21 mmol, 2 eq) was added and the reaction was heated at 80° C. for 18 h. After cooling, the mixture was partitioned between EtOAc and water, and the organic phase was washed with brine, dried (MgSO₄) and concentrated in vacuo. The residue was purified on a DCM-wet PE-AX cartridge (5 g), washed successively with DCM, MeOH and eluted with 5% HCOOH in DCM and concentrated in vacuo. Further purification by automated flash chromatography (CombiFlash Rf, 4 g RediSep™ silica cartridge) eluting with a gradient of 0-10% MeOH in DCM afforded Example 195 as a cream powder (6.8 mg, 0.01 mmol, 12%). LRMS calculated for C₃₂H₂₇ClN₂O₃S: 554; found: 555 (M+H). ¹H NMR (400 MHz, DMSO-d₆) δ ppm: 12.83 (br s, 1H), 8.50 (d, J=5.4 Hz, 1H), 8.01 (d, J=5.4 Hz, 1H), 7.63-7.58 (m, 1H), 7.51 (d, J=5.4 Hz, 1H), 7.41-7.36 (m, 1H), 7.32-7.23 (m, 2H), 7.04-6.98 (m, 2H), 6.93 (s, 1H), 6.64 (t, J=2.1 Hz, 1H), 6.58-6.52 (m, 2H), 6.33 (br s, 1H), 4.50 (t, J=6.9 Hz, 2H), 3.10 (t, J=6.9 Hz, 2H), 2.43-2.13 (m, 6H), 1.22-1.11 (m, 2H).

### Example 196 (1r,4r)-4-(3-chloroanilino)-2′-[3-(morpholin-4-yl)propyl]-2′,3′-dihydrospiro[cyclohexane-1,1′-indene]-4-carboxylic acid

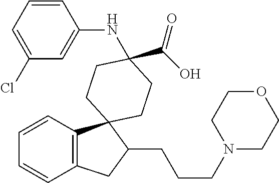

and

### Example 197 (1r,4r)-4-(3-chloroanilino)-2′-(3-methoxypropyl)-2′,3′-dihydrospiro[cyclohexane-1,1′-indene]-4-carboxylic acid

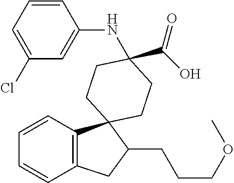

Using General procedure 40 and Preparation 9c as the appropriate mesylate and morpholine as the appropriate amine afforded an intermediate, which was hydrolysed according to General procedure 33b to obtain a mixture. The compounds were separated by preparative HPLC at pH4. The compound eluting earlier was further purified using a pre-wetted (MeOH) PE-AX cartridge (5 g), washing with MeOH, DCM and eluted with 10% HCOOH/DCM, to obtain Example 196, isolated as a white solid. LRMS calculated for C₂₈H₃₅ClN₂O₃: 482; found: 483 (M+H). ¹H NMR (400 MHz, DMSO-d₆) δ ppm: 7.40-7.33 (m, 1H), 7.22-7.10 (m, 3H), 7.07 (t, J=8.1 Hz, 1H), 6.63 (t, J=2.1 Hz, 1H), 6.58-6.52 (m, 2H), 6.23 (br s, 1H), 3.60-3.50 (m, 4H), 2.98 (dd, J=15.7, 7.2 Hz, 1H), 2.58-2.50 (m, 1H), 2.47-2.25 (m, 7H), 2.16-2.06 (m, 1H), 2.04-1.83 (m, 4H), 1.78-1.68 (m, 1H), 1.63-1.32 (m, 5H), 1.19-1.08 (m, 1H).

The compound eluting later was further purified using a pre-wetted (MeOH) PE-AX cartridge (5 g), washing with MeOH, DCM and eluted with 10% HCOOH/DCM, to obtain Example 197, isolated as a white solid. LRMS calculated for C₂₅H₃₀ClNO₃: 427; found 428 (M+H). ¹H NMR (400 MHz, DMSO-d₆) δ ppm: 7.40-7.33 (m, 1H), 7.23-7.11 (m, 3H), 7.06 (t, J=8.1 Hz, 1H), 6.63 (t, J=2.1 Hz, 1H), 6.58-6.52 (m, 2H), 6.20 (br s, 1H), 3.22 (s, 3H), 2.98 (dd, J=15.7, 7.2 Hz, 1H), 2.57-2.50 (m, 1H), 2.47-2.37 (m, 1H), 2.16-2.07 (m, 1H), 2.03-1.82 (m, 4H), 1.77-1.33 (m, 6H), 1.21-1.10 (m, 1H).

### Example 198 (1r,4r)-4-(3-chloroanilino)-2′-[3-(4-methyl-3-oxopiperazin-1-yl)propyl]-2′,3′-dihydrospiro[cyclohexane-1,1′-indene]-4-carboxylic acid

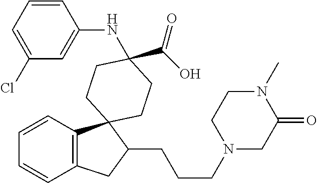

Using General procedure 40 and Preparation 9c as the appropriate mesylate and 1-methylpiperazin-2-one×HCl as the appropriate amine afforded an intermediate which was hydrolysed according to General procedure 33b. Purification by preparative HPLC at pH4 afforded a racemate, Example 198, as a solid. LRMS calculated for C₂₉H₃₆ClN₃O₃: 509; found: 510 (M+H). ¹H NMR (400 MHz, DMSO-d₆) δ ppm: 7.40-7.34 (m, 1H), 7.24-7.12 (m, 3H), 7.07 (t, J=8.1 Hz, 1H), 6.63 (t, J=2.2 Hz, 1H), 6.59-6.52 (m, 2H), 6.23 (br s, 1H), 3.00 (dd, J=15.6, 7.1 Hz, 1H), 2.84 (s, 3H), 2.55 (dd, J=16.6, 7.7 Hz, 1H), 2.47-2.36 (m, 1H), 2.17-2.07 (m, 1H), 2.04-1.83 (m, 4H), 1.78-1.61 (m, 2H), 1.59-1.33 (m, 4H), 1.23-1.09 (m, 1H).

### Example 199 (1r,4r)-4-(3-chloroanilino)-2′-[2-(pyridin-2-yl)ethyl]spiro[cyclohexane-1,1′-indene]-4-carboxylic acid

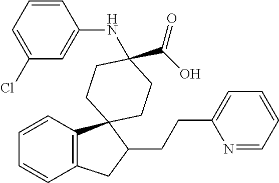

To an oven-dried microwave vial was added Preparation 6c (110 mg, 0.25 mmol, 1 eq), 2-(2-bromoethyl)pyridine×HCl (0.06 mL, 0.37 mmol, 1.5 eq), 4,4′-di-t-butyl-2,2′-bipyridine)bis[3,5-difluoro-2-[5-trifluoromethyl-2-pyridinyl-kN)phenyl-kC]iridium(III) hexafluorophosphate (3 mg, 2.46 μmol, 0.01 eq) and Na₂CO₃ (91 mg, 0.86 mmol, 3.5 eq). The mixture was sparged with N₂, followed by the addition of DME (2 mL) and 1,1,1,3,3,3-hexamethyl-2-(trimethylsilyl)trisilane (0.08 mL, 0.25 mmol, 1 eq). To a separate oven-dried microwave vial was added DME (1 mL), NiCl₂ (3 mg, 0.01 mmol, 0.05 eq) and 4,4′-di-tert-butyl-2,2′dipyridyl (3 mg, 0.01 mmol, 0.05 eq). The mixture was sparged with N₂ before the addition of DME (1 mL) and the mixture was stirred at. rt for 5 min. A solution of the nickel complex (0.1 mL) was added to the reaction mixture and then sparged with N₂. The mixture was irradiated with a 34 W blue LED lamp (450 nm), with compressed air cooling for 18 h. The reaction was partitioned between EtOAc and water, and the organic phase was washed with brine, dried (MgSO₄) and concentrated in vacuo. Purification by automated flash chromatography (CombiFlash Rf, 12 g RediSep™ silica cartridge) eluting with a gradient of 0-100% EtOAc in heptane afforded an intermediate, which was hydrolysed according to General procedure 33b to obtain Example 199 as a white solid (3 mg, 0.065 mmol, 3%). LRMS calculated for C₂₈H₂₇ClN₂O₂: 458; found: 459 (M+H). ¹H NMR (400 MHz, DMSO-d₆) δ ppm: 8.52-8.48 (m, 1H), 7.80 (d, J=7.6 Hz, 1H), 7.74-7.67 (m, 1H), 7.35 (d, J=7.8 Hz, 1H), 7.27-7.15 (m, 3H), 7.10-7.04 (m, 1H), 6.95-6.88 (m, 1H), 6.62-6.53 (m, 2H), 6.48-6.45 (m, 1H), 6.38-6.32 (m, 1H), 5.81 (br s, 1H), 3.12-3.05 (m, 2H), 2.67-2.60 (m, 2H), 2.46-2.34 (m, 2H), 2.12-1.96 (m, 4H), 0.83-0.73 (m, 2H).

### Example 200 (1r,4r)-4-(3-chloroanilino)-2′-(2-phenylethyl)spiro[cyclohexane-1,1′-indene]-4-carboxylic acid

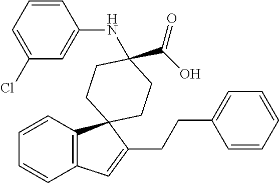 [SMILES: O=C(O)[C@]1(Nc2cccc(Cl)c2)CC[C@]2(CC1)C(CCc1ccccc1)=Cc1ccccc12]

To an oven-dried microwave vial was added Preparation 6c (118 mg, 0.26 mmol, 1 eq), (2-bromoethyl)benzene (0.05 mL, 0.4 mmol, 1.5 eq), 4,4′-di-t-butyl-2,2′-bipyridine)bis[3,5-difluoro-2-[5-trifluoromethyl-2-pyridinyl-kN)phenyl-kC]iridium(III) hexafluorophosphate (3 mg, 2.64 μmol, 0.01 eq) and Na₂CO₃ (56 mg, 0.53 mmol, 2 eq). The mixture was sparged with N₂ followed by the addition of DME (2 mL) and 1,1,1,3,3,3-hexamethyl-2-(trimethylsilyl)trisilane (0.08 mL, 0.26 mmol, 1 eq). To a separate oven-dried microwave vial was added DME (1 mL), NiCl₂ (3 mg, 0.01 mmol, 0.05 eq) and 4,4′-di-tert-butyl-2,2′dipyridyl (3.54 mg, 0.01 mmol, 0.05 eq). The mixture was sparged with N₂ before the addition of DME (1 mL) and the mixture was stirred at rt for 5 min. A solution of the nickel complex (0.1 mL) was added to the reaction mixture and then sparged with N₂. The mixture was irradiated with a 34 W blue LED lamp (450 nm), with compressed air cooling for 18 h. The reaction was partitioned between EtOAc and water, and the organic phase was washed with brine, dried (MgSO₄) and concentrated in vacuo. Purification by automated flash chromatography (CombiFlash Rf, 12 g RediSep™ silica cartridge) eluting with a gradient of 0-50% EtOAc in heptane, followed by reverse phase automated flash chromatography (CombiFlash Rf, C18 26 g RediSep column) eluting with a gradient of 10-100% MeCN in water afforded an intermediate, which was hydrolysed according to General procedure 33b to obtain Example 200 as a white solid (26.5 mg, 0.06 mmol, 22%). LRMS calculated for C₂₉H₂₈ClNO₂: 457; found: 458 (M+H). ¹H NMR (400 MHz, DMSO-d₆) δ ppm: 12.92 (br s, 1H), 7.67 (d, J=7.5 Hz, 1H), 7.34-7.27 (m, 5H), 7.26-7.17 (m, 2H), 7.15-7.04 (m, 2H), 6.64 (t, J=2.1 Hz, 1H), 6.58-6.52 (m, 3H), 6.35 (br s, 1H), 2.94 (dd, J=8.9, 7.0 Hz, 2H), 2.46-2.34 (m, 2H), 2.23-2.09 (m, 4H), 0.94-0.85 (m, 2H).

### Example 201

### Example 201A 2-[(E)-2-phenylethenyl]-1H-indene

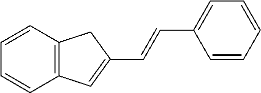 [SMILES: C1=C(/C=C/c2ccccc2)Cc2ccccc21]

To a solution of Example 148A (792 mg, 3.00 mmol, 1.0 eq) in a mixture of 1,4-dioxane (15 mL) and water (5 mL) was added Na₂CO₃ (954 mg, 9.0 mmol, 3 eq), [(E)-2-phenylethenyl]boronic acid (577 mg, 3.9 mmol, 1.3 eq) and Pd(PPh₃)₄ (173 mg, 0.15 mmol, 0.05 eq). The reaction was heated at 95° C. for 18 h and then allowed to cool to rt before being diluted with EtOAc (30 ml). The solids were separated via filtration and the filtrate was concentrated in vacuo. Purification by flash chromatography (40 g silica cartridge) eluting with PE afforded Example 201A as a colourless oil (500 mg, 2.39 mmol, 76%). ¹H NMR (400 MHz, CDCl₃) δ ppm: 7.51-7.40 (m, 3H), 7.38-7.30 (m, 3H), 7.28-7.14 (m, 4H), 6.84 (s, 1H), 6.78 (d, J=16.1 Hz, 1H), 3.66 (s, 2H).

### Example 201B 2″-[(E)-2-phenylethenyl]dispiro[[1,3]dioxolane-2,1′-cyclohexane-4′,1″-indene]

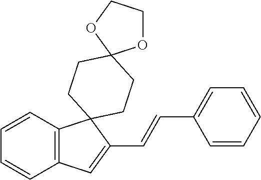 [SMILES: C1=C(/C=C/c2ccccc2)C2(CCC3(CC2)OCCO3)c2ccccc21]

Using General procedure 8a with Preparation 1a and Example 201A as the appropriate indene, Example 201B was obtained as a light yellow solid. ¹H NMR (400 MHz, CDCl₃) δ ppm: 7.73 (d, J=7.6 Hz, 1H), 7.52 (d, J=7.6 Hz, 2H), 7.40-7.32 (m, 3H), 7.32-7.21 (m, 2H), 7.19-7.08 (m, 2H), 6.99-6.90 (m, 2H), 4.08 (s, 4H), 2.50-2.37 (m, 2H), 2.35-2.23 (m, 2H), 1.97-1.86 (m, 2H), 1.41-1.30 (m, 2H).

### Example 201C 2″-(2-phenylethyl)-2″,3″-dihydrodispiro[[1,3]dioxolane-2,1′-cyclohexane-4′,1″-indene]

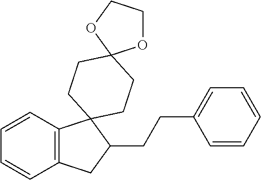 [SMILES: c1ccc(CCC2Cc3ccccc3C23CCC2(CC3)OCCO2)cc1]

Using General procedure 19 and Example 201B as the appropriate indene afforded a racemate, Example 201C, isolated as a colourless oil. ¹H NMR (400 MHz, CDCl₃) δ ppm: 7.37-7.09 (m, 9H), 3.98 (s, 4H), 3.11 (dd, J=15.7, 7.1 Hz, 1H), 2.86-2.67 (m, 2H), 2.60-2.48 (m, 1H), 2.28-2.17 (m, 1H), 2.00-1.89 (m, 1H), 1.88-1.70 (m, 6H), 1.68-1.40 (m, 3H).

### Example 201 4-(3-bromoanilino)-2′-(2-phenylethyl)-2′,3′-dihydrospiro[cyclohexane-1,1′-indene]-4-carboxylic acid

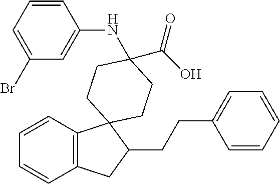 [SMILES: O=C(O)C1(Nc2cccc(Br)c2)CCC2(CC1)c1ccccc1CC2CCc1ccccc1]

Using General procedure 9 and Example 201C as the appropriate ketal afforded an intermediate which was treated according to General procedure 10 with 3-bromoaniline as the appropriate aniline to afford a racemic mixture of diastereoisomers, Example 201, isolated as an off-white solid (115 mg, 0.23 mmol, 26%). LRMS calculated for C₂₉H₃₀BrNO₂: 503; found: 504 (M+H). ¹H NMR (400 MHz, DMSO-d₆) δ ppm: 7.42-7.10 (m, 9H), 7.00/6.99 (t, J=8.1 Hz, 1H), 6.78/6.77 (t, J=2.1 Hz, 1H), 6.70-6.65 (m, 1H), 6.61-6.53 (m, 1H), 6.20 (br s, 1H), 3.12-2.97 (m, 1H), 2.82-2.19 (m, 4H), 2.11-1.20 (m, 10H).

### Example 202 and Example 203 Example 202A 2-methylindan-1-one

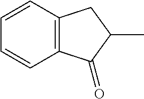

Indan-1-one (13.2 g, 100 mmol) was dissolved in dry THE (250 mL) and cooled to −78° C. under N₂. LDA in THF/heptane/ethylbenzene (2 M, 50 mL, 100 mmol) was added to the mixture dropwise at −78° C., and stirred at −78° C. for 30 min. Then the cooling bath was removed and the mixture was stirred for 1 h. The mixture was cooled back to −78° C. under N₂, MeI (9.34 mL, 150 mmol) was added to the mixture dropwise, the mixture was allowed to warm to rt and stirred at rt for 2 h. The reaction mixture was diluted with sat. aq. NH₄Cl solution and extracted with EtOAc. The combined organic layers were washed with brine, then dried over Na₂SO₄, filtered and the filtrate was concentrated under reduced pressure. The crude product was purified via flash chromatography using heptane and EtOAc as eluents to obtain Example 202A. MS (EI, 70 eV) m/z (% relative intensity, [ion]): 103 (27), 117 (34), 131 (100), 146 (60, [M+]).

### Example 202B 2,3-dimethyl-1H-indene

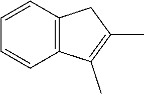

Example 202A (2.2 g, 15 mmol) was dissolved in Et₂O (50 mL) and cooled to 0° C. under N₂. MeMgCl in THE (3M, 7.5 mL, 22.5 mmol) was added to the mixture dropwise and stirred at rt for 1 h. The reaction mixture was diluted with sat. aq. NH₄Cl solution and extracted with Et₂O. The combined organic layers were washed with brine, then dried over Na₂SO₄, filtered and the filtrate was concentrated under reduced pressure. The residue was taken up in dry THE (50 mL), PTSA (40 mg, 0.2 mmol) was added to the mixture and stirred at reflux temperature for 2 h. The mixture was concentrated under reduced pressure and purified via flash chromatography using heptane and EtOAc as eluents to obtain Example 202B. MS (EI, 70 eV) m/z (% relative intensity, [ion]): 115 (13), 128 (51), 129 (100), 144 (50, [M+]).

### Example 202C 2″,3″-dimethyldispiro[[1,3]dioxolane-2,1′-cyclohexane-4′,1″-indene]

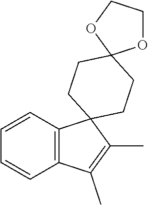 [SMILES: CC1=C(C)C2(CCC3(CC2)OCCO3)c2ccccc21]

Using General procedure 8a and Example 202B as the appropriate indene, Example 202C was obtained. ¹H NMR (500 MHz, DMSO-d₆) δ ppm: 7.61 (d, 1H), 7.25 (t, 1H), 7.22 (dd, 1H), 7.1 (td, 1H), 3.95 (s, 4H), 2.11/1.8 (td+d, 4H), 2.02/1.01 (td+d, 4H), 1.97 (s, 3H), 1.84 (s, 3H). HRMS calculated for C₁₈H₂₂O₂: 270.1620; found 271.1695 (M+H).

### Example 202D 2′,3′-dimethylspiro[cyclohexane-4,1′-indene]-1-one

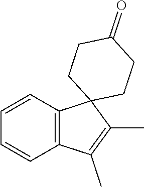 [SMILES: CC1=C(C)C2(CCC(=O)CC2)c2ccccc21]

Using General procedure 9 and Example 202C as the appropriate ketal, Example 202D was obtained. ¹H NMR (500 MHz, DMSO-d₆) δ ppm: 7.83 (dm, 1H), 7.29 (m, 1H), 7.26 (dm, 1H), 7.12 (m, 1H), 2.94 (m+m, 4H), 2.21/1.36 (m+m, 4H), 1.99 (s, 3H), 1.87 (s, 3H). HRMS calculated for C₁₆H₁₈O: 226.1358; found 227.1433 (M+H).

### Example 202E 2″,3″-dimethyldispiro[imidazolidine-4,1′-cyclohexane-4′,1″-indene]-2,5-dione

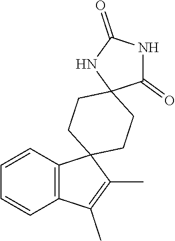 [SMILES: CC1=C(C)C2(CCC3(CC2)NC(=O)NC3=O)c2ccccc21]

Using General procedure 14 and Example 202D as the appropriate ketone, Example 202E was obtained as a mixture of diastereoisomers. ¹H NMR (500 MHz, DMSO-d₆) δ ppm: 10.76/10.58 (s, 1H), 8.98/8.21 (s, 1H), 7.65/7.80 (d, 1H), 7.32-7.06 (m, 3H), 2.37/2.11 (td, 2H), 2.06/2.23 (t, 2H), 1.67/2.00 (d, 2H), 1.97 (s, 3H), 1.91/1.94 (s, 3H), 0.96/1.19 (d, 2H). HRMS calculated for C₁₈H₂₀N₂O₂: 296.1525; found 295.1474 and 295.1455 (M−H).

### Example 202F 1-amino-2′,3′-dimethyl-spiro[cyclohexane-4,1′-indene]-1-carboxylic acid

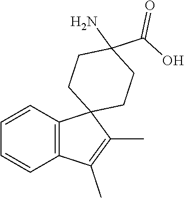 [SMILES: CC1=C(C)C2(CCC(N)(C(=O)O)CC2)c2ccccc21]

Using General procedure 15 and Example 202E as the appropriate hydantoin, Example 202F was obtained as a mixture of diastereoisomers. ¹H NMR (500 MHz, DMSO-d₆) δ ppm: 7.96 (dm, 1H), 7.87 (br s, 3H), 7.25 (m, 1H), 7.21 (dm, 1H), 7.09 (m, 1H), 2.65/1.75 (m+m, 4H), 2/0.87 (m+m, 4H), 1.96 (s, 3H), 1.9 (s, 3H). HRMS calculated for C₁₇H₂₁NO₂: 271.1572; found 272.1648 and 272.1648 (M+H).

### Example 202 (1r,4r)-4-(3-bromoanilino)-2′,3′-dimethylspiro[cyclohexane-1,1′-indene]-4-carboxylic acid

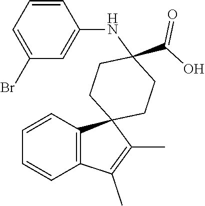 [SMILES: CC1=C(C)[C@]2(CC[C@](Nc3cccc(Br)c3)(C(=O)O)CC2)c2ccccc21]

and

### Example 203 (1s,4s)-4-(3-bromoanilino)-2′,3′-dimethylspiro[cyclohexane-1,1′-indene]-4-carboxylic acid

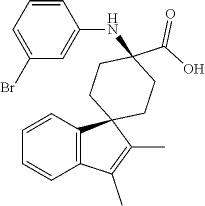 [SMILES: CC1=C(C)[C@]2(CC[C@@](Nc3cccc(Br)c3)(C(=O)O)CC2)c2ccccc21]

Using General procedure 16 and Example 202F as the appropriate amino acid, a mixture of diastereoisomers was obtained. The diastereoisomers were separated by chiral chromatography. Column: AD, 50×500 mm, 20 μm, Eluents: 15:85 EtOH/heptane+0.05% HCOOH. The diastereoisomer eluting earlier was collected as Example 203. ¹H NMR (500 MHz, DMSO-d₆) δ ppm: 7.66 (d, 1H), 7.25 (t, 1H), 7.22 (d, 1H), 7.11 (t, 1H), 7.02 (t, 1H), 6.86 (br s, 1H), 6.7 (d, 1H), 6.67 (d, 1H), 2.49/2.09 (d+t, 4H), 1.97 (s, 3H), 1.95/1.16 (t+d, 4H), 1.85 (s, 3H). HRMS calculated for C₂₃H₂₄BrNO₂: 425.099; found 426.1051 (M+H).

The diastereoisomer eluting later was collected as Example 202. ¹H NMR (500 MHz, DMSO-d₆) δ ppm: 12.81 (br s, 1H), 7.65 (dm, 1H), 7.26 (m, 1H), 7.23 (dm, 1H), 7.13 (m, 1H), 7.02 (t, 1H), 6.8 (t, 1H), 6.69 (dm, 1H), 6.59 (dm, 1H), 6.33 (s, 1H), 2.39/2.18 (m+m, 4H), 2.1/0.83 (m+m, 4H), 1.97 (s, 3H), 1.84 (s, 3H). HRMS calculated for C₂₃H₂₄BrNO₂: 425.099; found 426.1061 (M+H).

### Example 204

### Example 204A 3-isopropyl-2-methyl-1H-indene

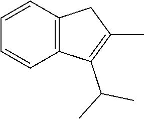

Example 202A (2.2 g, 15 mmol) was dissolved in Et₂O (50 mL) and cooled to 0° C. under N₂. iPrMgCl in THE (2 M, 11.3 mL, 22.5 mmol) was added to the mixture dropwise and stirred at rt for 2 h. The reaction mixture was diluted with sat. aq. NH₄Cl solution and extracted with Et₂O. The combined organic layers were washed with brine, then dried over Na₂SO₄, filtered and the filtrate was concentrated under reduced pressure. The residue was taken up in dry THE (50 mL), PTSA (40 mg, 0.2 mmol) was added to the mixture and stirred at reflux temperature for 2 h. The mixture was concentrated under reduced pressure and purified via flash chromatography using heptane and EtOAc as eluents to obtain Example 204A. ¹H NMR (500 MHz, DMSO-d₆) δ ppm: 7.4 (d, 1H), 7.34 (d, 1H), 7.18 (t, 1H), 7.05 (td, 1H), 3.24 (br s, 2H), 3.11 (sept., 1H), 2.05 (s, 3H), 1.28 (d, 6H). HRMS calculated for C₁₃H₁₆: 172.1252; found 172.12626 (M+).

### Example 204B 2″-methyl-3″-(propan-2-yl)dispiro[[1,3]dioxolane-2,1′-cyclohexane-4′,1″-indene]

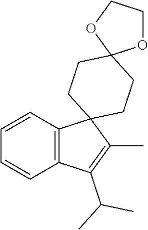 [SMILES: CC1=C(C(C)C)c2ccccc2C12CCC1(CC2)OCCO1]

Using General procedure 8b and Example 204A as the appropriate indene, Example 204B was obtained. ¹H NMR (500 MHz, DMSO-d₆) δ ppm: 7.62 (d, 1H), 7.42 (d, 1H), 7.21 (t, 1H), 7.07 (t, 1H), 3.95-3.93 (m, 4H), 3.08 (sept., 1H), 2.11/1.78 (m, 4H), 2.01/0.98 (m, 4H), 1.84 (s, 3H), 1.27 (d, 6H). HRMS calculated for C₂₀H₂₆O₂: 298.1933; found 299.2013 (M+H).

### Example 204C 3′-isopropyl-2′-methyl-spiro[cyclohexane-4,1′-indene]-1-one

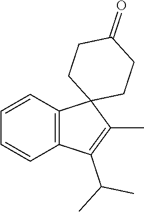 [SMILES: CC1=C(C(C)C)c2ccccc2C12CCC(=O)CC2]

Using General procedure 9 and Example 204B as the appropriate ketal, Example 204C was obtained. ¹H NMR (500 MHz, DMSO-d₆) δ ppm: 7.83 (d, 1H), 7.46 (d, 1H), 7.25 (t, 1H), 7.09 (t, 1H), 3.1 (sept., 1H), 2.93/2.38 (m, 4H), 2.21/1.34 (m, 4H), 1.88 (s, 3H), 1.28 (d, 6H). HRMS calculated for C₁₈H₂₂O: 254.1671; found 255.1748 (M+H).

### Example 204D 2″-methyl-3″-(propan-2-yl)dispiro[imidazolidine-4,1′-cyclohexane-4′,1″-indene]-2,5-dione

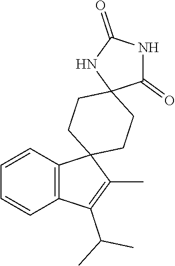 [SMILES: CC1=C(C(C)C)c2ccccc2C12CCC1(CC2)NC(=O)NC1=O]

Using General procedure 14 and Example 204C as the appropriate ketone, a mixture of diastereoisomers was obtained. The crude product was purified via flash chromatography using DCM and MeOH as eluents to obtain Example 204D as a mixture of diastereoisomers. ¹H NMR (500 MHz, DMSO-d₆) δ ppm: 10.74 (br s, 1H), 8.97 (s, 1H), 7.68 (dm, 1H), 7.43 (dm, 1H), 7.23 (m, 1H), 7.11 (m, 1H), 3.08 (sp, 1H), 2.37/1.66 (m+m, 4H), 2.04/0.94 (m+m, 4H), 1.92 (s, 3H), 1.27 (d, 6H). HRMS calculated for C₂₀H₂₄N₂O₂: 324.1838; found 325.1916 (M+H).

### Example 204E 4-amino-2′-methyl-3′-(propan-2-yl)spiro[cyclohexane-1,1′-indene]-4-carboxylic acid

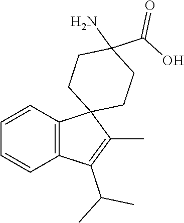 [SMILES: CC1=C(C(C)C)c2ccccc2C12CCC(N)(C(=O)O)CC2]

Using General procedure 15 and Example 204D as the appropriate hydantoin, Example 204E was obtained as a mixture of diastereoisomers. ¹H NMR (500 MHz, DMSO-d₆) δ ppm: 8.21 (br s, 3H), 7.86 (dm, 1H), 7.42 (dm, 1H), 7.22 (m, 1H), 7.08 (m, 1H), 3.09 (sp, 1H), 2.62/1.85 (m+m, 4H), 2/0.92 (m+m, 4H), 1.93 (s, 3H), 1.27 (d, 6H). HRMS calculated for C₁₉H₂₅NO₂: 299.1885; found 300.1962 (M+H).

### Example 204 (1r,4r)-4-(3-bromoanilino)-2′-methyl-3′-(propan-2-yl)spiro[cyclohexane-1,1′-indene]-4-carboxylic acid

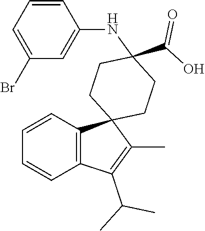 [SMILES: CC1=C(C(C)C)c2ccccc2[C@]12CC[C@@](Nc1cccc(Br)c1)(C(=O)O)CC2]

Using General procedure 16 and Example 204E as the appropriate amino acid and 1-bromo-3-iodo-benzene as the appropriate iodobenzene, a mixture of diastereoisomers was obtained. The diastereoisomers were separated by chiral chromatography. Column: AD, 50×500 mm, 20 μm, Eluents: 50:50 EtOH/heptane+0.05% HCOOH. The diastereoisomer eluting later was collected and purified via prep RP-HPLC using 25 mM aq. NH₄HCO₃ solution and MeCN as eluents resulting Example 204. ¹H NMR (500 MHz, DMSO-d₆) δ ppm: 7.69 (d, 1H), 7.41 (d, 1H), 7.21 (t, 1H), 7.08 (t, 1H), 6.99 (t, 1H), 6.8 (t, 1H), 6.65 (dm, 1H), 6.59 (dm, 1H), 6.28 (br, 1H), 3.07 (sp, 1H), 2.38/2.14 (td+d, 4H), 2.06/0.78 (td+d, 4H), 1.84 (s, 3H), 1.26 (d, 6H). HRMS calculated for C₂₅H₂₈NO₂Br: 453.1303; found: 454.1375 (M+H).

### Example 205 and Example 206

### Example 205A 3-ethyl-2-methyl-1H-indene

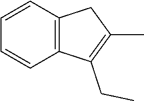

Example 202A (2.2 g, 15 mmol) was dissolved in Et₂O (50 mL) and cooled to 0° C. under N₂. EtMgCl in THF (2 M, 11.3 mL, 22.5 mmol) was added to the mixture dropwise and stirred at rt for 1 h. The reaction mixture was diluted with sat. aq. NH₄Cl solution and extracted with Et₂O. The combined organic layers were washed with brine, then dried over Na₂SO₄, filtered and the filtrate was concentrated under reduced pressure. The residue was taken up in dry THE (50 mL), PTSA (40 mg, 0.2 mmol) was added to the mixture and stirred at reflux temperature for 2 h. The mixture was concentrated under reduced pressure and purified via flash chromatography using heptane and EtOAc as eluents to obtain Example 205A. ¹H NMR (500 MHz, DMSO-d₆) δ ppm: 7.35 (d, 1H), 7.22 (d, 1H), 7.22 (t, 1H), 7.06 (t, 1H), 3.25 (s, 2H), 2.48 (q, 2H), 2.03 (s, 3H), 1.08 (t, 3H). MS (EI, 70 eV) m/z (% relative intensity, [ion]): 115 (29), 128 (73), 143 (100), 158 (48, [M+]).

### Example 205B 3″-ethyl-2″-methyldispiro[[1,3]dioxolane-2,1′-cyclohexane-4′,1″-indene]

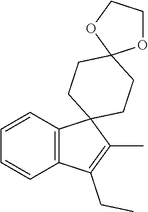 [SMILES: CCC1=C(C)C2(CCC3(CC2)OCCO3)c2ccccc21]

Using General procedure 8b and Example 205A as the appropriate indene, Example 205B was obtained. ¹H NMR (500 MHz, DMSO-d₆) δ ppm: 7.61 (dm, 1H), 7.26 (dm, 1H), 7.24 (m, 1H), 7.09 (m, 1H), 3.99-3.9 (m, 4H), 2.45 (q, 2H), 2.11/1.80 (m+m, 4H), 2.02/1.00 (m+m, 4H), 1.83 (s, 3H), 1.05 (t, 3H). HRMS calculated for C₁₉H₂₄O₂: 284.1776; found 285.1848 (M+H).

### Example 205C 3′-ethyl-2′-methylspiro[cyclohexane-1,1′-inden]-4-one

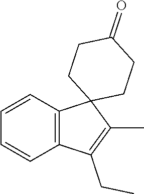 [SMILES: CCC1=C(C)C2(CCC(=O)CC2)c2ccccc21]

Using General procedure 9 and Example 205B as the appropriate ketal, Example 205C was obtained. ¹H NMR (500 MHz, DMSO-d₆) δ ppm: 7.83 (d, 1H), 7.29 (m, 1H), 7.27 (m, 1H), 7.11 (t, 1H), 2.94/2.38 (m, 4H), 2.48 (m, 2H), 2.21/1.36 (m, 4H), 1.87 (s, 3H), 1.07 (t, 3H). HRMS calculated for C₁₇H₂₀O: 240.1514; found 241.1590 (M+H).

### Example 205D 2″-methyl-3″-(propan-2-yl)dispiro[imidazolidine-4,1′-cyclohexane-4′,1″-indene]-2,5-dione

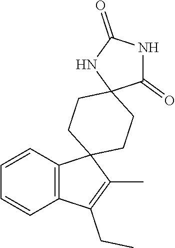 [SMILES: CCC1=C(C)C2(CCC3(CC2)NC(=O)NC3=O)c2ccccc21]

Using General procedure 14 and Example 205C as the appropriate ketone, a mixture of diastereoisomers was obtained. The crude product was purified via flash chromatography using DCM and MeOH as eluents to obtain Example 205D as a mixture of diastereoisomers. ¹H NMR (500 MHz, DMSO-d₆) δ ppm: 10.76 (s, 1H), 8.98 (s, 1H), 7.66 (dm, 1H), 7.3-7.09 (m, 3H), 2.46 (q, 2H), 2.37/1.67 (m+m, 4H), 2.06/0.96 (m+m, 4H), 1.90 (s, 3H), 1.06 (t, 3H). HRMS calculated for C₁₉H₂₂N₂O₂: 310.1681; found 311.1746 (M+H).

### Example 205E 4-amino-3′-ethyl-2′-methylspiro[cyclohexane-1,1′-indene]-4-carboxylic acid H₂N

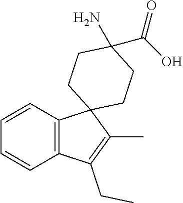 [SMILES: CCC1=C(C)C2(CCC(N)(C(=O)O)CC2)c2ccccc21]

Using General procedure 15 and Example 205D as the appropriate hydantoin, Example 205E was obtained as a mixture of diastereoisomers. ¹H NMR (500 MHz, DMSO-d₆) δ ppm: 7.93 (d, 1H), 7.24 (m, 1H), 7.24 (m, 1H), 7.08 (m, 1H), 2.64/1.77 (td+d, 4H), 2.45 (q, 2H), 1.99/0.89 (td+d, 4H), 1.89 (s, 3H), 1.06 (t, 3H). HRMS calculated for C₁₈H₂₃NO₂: 285.1729; found 286.1806 (M+H).

### Example 205 (1r,4r)-4-(3-chloroanilino)-3′-ethyl-2′-methylspiro[cyclohexane-1,1′-indene]-4-carboxylic acid

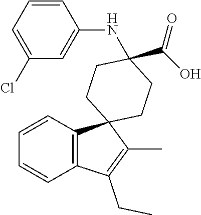 [SMILES: CCC1=C(C)[C@]2(CC[C@](Nc3cccc(Cl)c3)(C(=O)O)CC2)c2ccccc21]

and

### Example 206 (1s,4s)-4-(3-chloroanilino)-3′-ethyl-2′-methylspiro[cyclohexane-1,1′-indene]-4-carboxylic acid

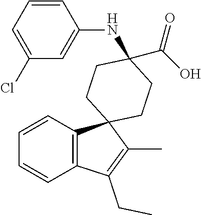 [SMILES: CCC1=C(C)[C@]2(CC[C@@](Nc3cccc(Cl)c3)(C(=O)O)CC2)c2ccccc21]

Using General procedure 16 and Example 205E as the appropriate amino acid and 1-chloro-3-iodo-benzene as the appropriate iodobenzene, a mixture of diastereoisomers was obtained. The diastereoisomers were separated by chiral chromatography. Column: AD, 50×500 mm, 20 μm, Eluents: 50:50 EtOH/heptane+0.05% HCOOH. The diastereoisomer eluting earlier was collected as Example 206. ¹H NMR (500 MHz, DMSO-d₆) δ ppm: 12.88 (br, 1H), 7.66 (d, 1H), 7.34 (d, 1H), 7.26 (t, 1H), 7.19 (t, 1H), 7.07 (t, 1H), 6.71 (t, 1H), 6.63 (dd, 1H), 6.56 (d, 1H), 6.39 (br, 1H), 2.49/2.08 (br d+br t, 4H), 2.46 (q, 2H), 1.95/1.15 (br t+br d, 4H), 1.84 (s, 3H), 1.06 (t, 3H). HRMS calculated for C₂₄H₂₆NO₂Cl: 395.1652; found: 396.1721 (M+H).

The diastereoisomer eluting later was collected and purified via prep RP-HPLC using 25 mM aq. NH₄HCO₃ solution and MeCN as eluents resulting in Example 205. ¹H NMR (500 MHz, DMSO-d₆) δ ppm: 12.84 (br s, 1H), 7.66 (d, 1H), 7.26 (m, 1H), 7.26 (m, 1H), 7.11 (t, 1H), 7.08 (t, 1H), 6.64 (dd, 1H), 6.57 (dd, 1H), 6.56 (dd, 1H), 6.34 (br s, 1H), 2.45 (q, 2H), 2.39/2.18 (t+d, 4H), 2.09/0.82 (t+d, 4H), 1.83 (s, 3H), 1.05 (t, 3H). HRMS calculated for C₂₄H₂₆NO₂Cl: 395.1652; found: 396.1721 (M+H).

### Example 207 (1r,2′RS,3′SR,4r)-4-(3-chloroanilino)-3′-ethyl-2′-methyl-2′,3′-dihydrospiro[cyclohexane-1,1′-indene]-4-carboxylic acid

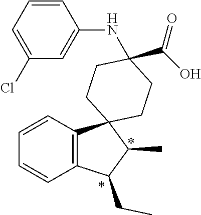 [SMILES: CC[C@H]1c2ccccc2[C@]2(CC[C@@](Nc3cccc(Cl)c3)(C(=O)O)CC2)[C@H]1C]

Example 205 (19 mg, 0.05 mmol) was dissolved in THE (1 mL) and AcOH (50 μL). 10% Pt/C (9 mg) was added to the mixture. The autoclave was evacuated and backfilled with N₂ (3 times), then evacuated and filled with H₂ (10 bar). The reaction mixture was stirred at rt until no further conversion was observed. The reaction mixture was filtered, washed with EtOAc, and concentrated under reduced pressure. The crude product was purified via prep RP-HPLC using 25 mM aq. NH₄HCO₃ solution and MeCN as eluents resulting in Example 207 as a racemate. ¹H NMR (500 MHz, DMSO-d₆) δ: 7.19-7.09 (m, 4H), 7.02 (t, 1H), 6.68 (br s, 1H), 6.58 (d, 1H), 6.51 (br d, 1H), 6.10 (br, 1H), 3.09 (m, 1H), 2.48 (m, 1H), 2.44-1.36 (m, 8H), 1.91/1.37 (m+m, 2H), 1.03 (t, 3H), 0.63 (d, 3H). HRMS calculated for C₂₄H₂₈NO₂Cl: 397.1808; found: 398.1878 (M+H).

### Example 208

### Example 208A rel-(2″R,3″S)-2″,3″-dimethyl-2″,3″-dihydrodispiro[[1,3]dioxolane-2,1′-cyclohexane-4′,1″-indene], enantiomer 1

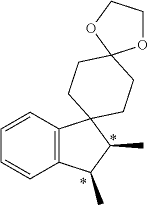 [SMILES: C[C@H]1c2ccccc2C2(CCC3(CC2)OCCO3)[C@H]1C]

Example 202C (1.1 g, 4.1 mmol) was dissolved in THF (20 mL). 10% Pt/C (50 mg) was added to the mixture. The flask was evacuated and backfilled with N₂ (3 times), then evacuated and filled with H₂. The reaction mixture was stirred at rt until no further conversion was observed. The reaction mixture was filtered, washed with EtOAc, and concentrated under reduced pressure to obtain a racemate. The enantiomers were separated by chiral chromatography. Column: OD, 50×500 mm, 20 μm, Eluents: 50:50 EtOH/heptane. The enantiomer eluting earlier was collected as Example 208A.

### Example 208B rel-(2′R,3'S)-2′,3′-dimethyl-2′,3′-dihydrospiro[cyclohexane-1,1′-inden]-4-one, enantiomer 1

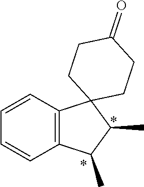 [SMILES: C[C@H]1c2ccccc2C2(CCC(=O)CC2)[C@H]1C]

Using General procedure 9 and Example 208A as the appropriate ketal, Example 208B was obtained. ¹H NMR (500 MHz, DMSO-d₆) δ ppm: 7.20 (m, 1H), 7.17 (m, 2H), 7.16 (m, 1H), 3.43 (quint, 1H), 2.73 (quint, 1H), 2.70-1.53 (m, 8H), 1.23 (d, 3H), 0.73 (d, 3H). HRMS calculated for C₁₆H₂₀O: 228.1514; found: 229.1590 (M+H).

### Example 208C rel-(2″R,3″S)-2″,3″-dimethyl-2″,3″-dihydrodispiro[imidazolidine-4,1′-cyclohexane-4′,1″-indene]-2,5-dione, enantiomer 1

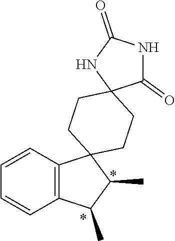 [SMILES: C[C@H]1c2ccccc2C2(CCC3(CC2)NC(=O)NC3=O)[C@H]1C]

Using General procedure 14 and Example 208B as the appropriate ketone, a mixture of diastereoisomers was obtained. The crude product was purified via flash chromatography using DCM and MeOH as eluents to obtain Example 208C as a mixture of diastereoisomers. ¹H NMR (500 MHz, DMSO-d₆) δ ppm: 10.62 (br, 1H), 8.78 (s, 1H), 7.34-7.03 (m, 4H), 3.35 (m, 1H), 2.49 (m, 1H), 1.19 (d, 3H), 0.63 (d, 3H). HRMS calculated for C₁₈H₂₂N₂O₂: 298.1681; found 299.1756 (M+H).

### Example 208D rel-(2′R,3'S)-4-amino-2′,3′-dimethyl-2′,3′-dihydrospiro[cyclohexane-1,1′-indene]-4-carboxylic acid, enantiomer 1

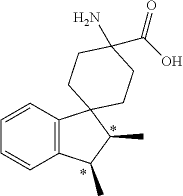 [SMILES: C[C@H]1c2ccccc2C2(CCC(N)(C(=O)O)CC2)[C@H]1C]

Using General procedure 15 and Example 208C as the appropriate hydantoin, Example 208D was obtained as a mixture of diastereoisomers. LRMS calculated for C₁₇H₂₃NO₂: 273.2; found 274.4 (M+H).

### Example 208 (1r,2′R*,3'S*,4r)-4-(3-chloroanilino)-2′,3′-dimethyl-2′,3′-dihydrospiro[cyclohexane-1,1′-indene]-4-carboxylic acid, enantiomer 1

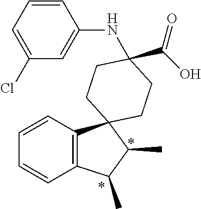 [SMILES: C[C@H]1c2ccccc2[C@]2(CC[C@@](Nc3cccc(Cl)c3)(C(=O)O)CC2)[C@H]1C]

Using General procedure 16 and Example 208D as the appropriate amino acid and 1-chloro-3-iodo-benzene as the appropriate iodobenzene, a mixture of diastereoisomers was obtained. The diastereoisomers were separated via flash chromatography using heptane and EtOAc as eluents. The diastereoisomer eluting later was collected as Example 208. ¹H NMR (500 MHz, DMSO-d₆) δ ppm: 12.63 (br s, 1H), 7.16-7.07 (m, 4H), 7.06 (t, 1H), 6.61 (t, 1H), 6.55 (dd, 1H), 6.53 (dd, 1H), 6.24 (s, 1H), 3.35 (m, 1H), 2.52 (m, 1H), 2.12-1.45 (m, 8H), 1.19 (d, 3H), 0.64 (d, 3H). HRMS calculated for C₂₃H₂₆NO₂Cl: 383.1652; found: 384.1731 (M+H).

### Example 209

### Example 209A 2″-phenyldispiro[[1,3]dioxolane-2,1′-cyclohexane-4′,1″-indene]

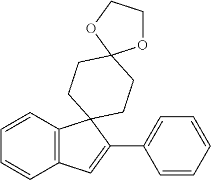 [SMILES: C1=C(c2ccccc2)C2(CCC3(CC2)OCCO3)c2ccccc21]

Using General procedure 8a and 2-phenyl-1H-indene (0.75 g, 3.9 mmol, 1 eq) as the appropriate indene and Preparation 1a (1.24 g, 3.9 mmol, 1 eq) afforded Example 209A, isolated as an orange solid (686 mg, 2.15 mmol, 55%). ¹H NMR (400 MHz, DMSO-d₆) δ ppm: 7.76-7.71 (m, 1H), 7.63-7.58 (m, 2H), 7.47-7.40 (m, 3H), 7.38-7.33 (m, 1H), 7.33-7.27 (m, 1H), 7.20 (td, J=7.5, 1.3 Hz, 1H), 7.05 (s, 1H), 3.95 (s, 4H), 2.37 (td, J=13.6, 4.4 Hz, 2H), 2.16 (td, J=13.6, 4.5 Hz, 2H), 1.85-1.76 (m, 2H), 1.32-1.23 (m, 2H).

### Example 209B 2′-phenylspiro[cyclohexane-1,1′-inden]-4-one

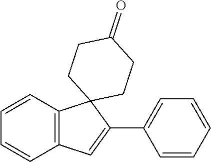 [SMILES: O=C1CCC2(CC1)C(c1ccccc1)=Cc1ccccc12]

To a solution of Example 209A (110 mg, 0.35 mmol, 1 eq) in THE (3 mL) was added 2 M aq. HCl solution (1.04 mL, 2.07 mmol, 6 eq) and the mixture was heated at 40° C. for 5 h in a sealed flask. Then it was allowed to cool to rt and partitioned between EtOAc and sat. aq. NaHCO₃ solution. The phases were separated and the organic phase was washed with brine, dried (MgSO₄) and concentrated in vacuo to afford Example 209B as an orange solid (95 mg, 0.35 mmol, 100%) that was used directly in the subsequent step without further purification. ¹H NMR (400 MHz, DMSO-d₆) δ ppm: 7.92-7.87 (m, 1H), 7.52-7.40 (m, 5H), 7.40-7.31 (m, 2H), 7.22 (td, J=7.5, 1.3 Hz, 1H), 6.98 (d, J=0.6 Hz, 1H), 3.01-2.88 (m, 2H), 2.48-2.35 (m, 4H), 1.71-1.61 (m, 2H).

### Example 209 (1r,4r)-4-(3-bromoanilino)-2′-phenylspiro[cyclohexane-1,1′-indene]-4-carboxylic acid

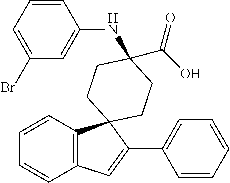 [SMILES: O=C(O)[C@]1(Nc2cccc(Br)c2)CC[C@@]2(CC1)C(c1ccccc1)=Cc1ccccc12]

Using General Procedure 10 and Example 209B (99 mg, 0.36 mmol 1 eq) as the appropriate ketone and 3-bromoaniline (39 μL, 0.36 mmol, 1 eq) as the appropriate aniline, a mixture of diastereoisomers was obtained. Purification by flash chromatography (5 g silica cartridge) eluting with 50% DCM in heptane, DCM and then 50% EtOAc in DCM afforded the major isomer as a single diastereoisomer, Example 209, isolated as an orange foam (31.3 mg, 0.06 mmol, 18%). LRMS calculated for C₂₇H₂₄BrNO₂: 473; found: 474 (M+H). ¹H NMR (400 MHz, DMSO-d₆) δ ppm: 7.78 (d, J=7.6 Hz, 1H), 7.58-7.53 (m, 2H), 7.44-7.27 (m, 5H), 7.24-7.19 (m, 1H), 7.02 (t, J=8.1 Hz, 1H), 7.00 (s, 1H), 6.84 (t, J=2.1 Hz, 1H), 6.73-6.69 (m, 1H), 6.68-6.64 (m, 1H), 2.48-2.40 (m, 2H), 2.37-2.26 (m, 2H), 2.20-2.09 (m, 2H), 1.44-1.36 (m, 2H).

### Example 210 and Example 211

### Example 210A 2″-phenyl-2″,3″-dihydrodispiro[[1,3]dioxolane-2,1′-cyclohexane-4′,1″-indene]

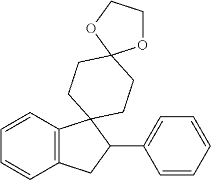 [SMILES: c1ccc(C2Cc3ccccc3C23CCC2(CC3)OCCO2)cc1]

To a solution of Example 209A (120 mg, 0.38 mmol, 1 eq) in EtOH (3 mL) and THE (3 mL) was added 10% Pd/C (40 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1), then the combined filtrates were concentrated in vacuo to afford a racemate, Example 210A, isolated as a pale yellow solid (112 mg, 0.35 mmol, 93%). ¹H NMR (400 MHz, DMSO-d₆) δ ppm: 7.31-7.13 (m, 7H), 7.08-6.99 (m, 2H), 3.90-3.77 (m, 4H), 3.54 (dd, J=7.7, 3.5 Hz, 1H), 3.43 (dd, J=16.1, 7.7 Hz, 1H), 2.94 (dd, J=16.1, 3.5 Hz, 1H), 1.96-1.73 (m, 4H), 1.67-1.57 (m, 1H), 1.44-1.22 (m, 3H).

### Example 210B 2′-phenyl-2′,3′-dihydrospiro[cyclohexane-1,1′-inden]-4-one

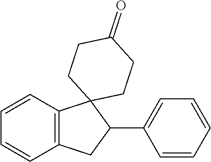 [SMILES: O=C1CCC2(CC1)c1ccccc1CC2c1ccccc1]

To a solution of Example 210A (112 mg, 0.35 mmol, 1 eq) in THE (3 mL) was added 2 M aq. HCl solution (1.05 mL, 2.09 mmol, 6 eq) and the mixture was heated at 40° C. for 4 h in a sealed flask. The mixture was allowed to cool to rt and partitioned between EtOAc and sat. aq. NaHCO₃ solution. The phases were separated, and the aq. phase was extracted with EtOAc. The combined organic extracts were washed with brine, dried (MgSO₄) and concentrated in vacuo to afford a racemate, Example 210B, isolated as a yellow solid (89.1 mg, 0.32 mmol, 92%) that was used directly in the subsequent step without further purification. ¹H NMR (400 MHz, DMSO-d₆) δ ppm: 7.40-7.29 (m, 2H), 7.29-7.12 (m, 7H), 3.74 (dd, J=7.7, 4.0 Hz, 1H), 3.50 (dd, J=16.2, 7.7 Hz, 1H), 3.04 (dd, J=16.2, 4.0 Hz, 1H), 2.83-2.71 (m, 1H), 2.46-2.36 (m, 1H), 2.16-1.97 (m, 4H), 1.93-1.83 (m, 1H), 1.56-1.44 (m, 1H).

### Example 210 (1s,4s)-4-(3-bromoanilino)-2′-phenyl-2′,3′-dihydrospiro[cyclohexane-1,1′-indene]-4-carboxylic acid, enantiomer 2

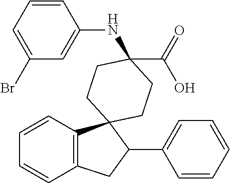

and

### Example 211 (1r,2′R,4R)-4-(3-bromoanilino)-2′-phenyl-2′,3′-dihydrospiro[cyclohexane-1,1′-indene]-4-carboxylic acid

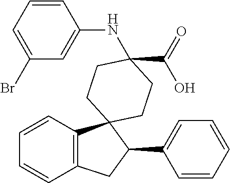 [SMILES: O=C(O)[C@]1(Nc2cccc(Br)c2)CC[C@@]2(CC1)c1ccccc1C[C@@H]2c1ccccc1]

Using General Procedure 10 and Example 210B (89 mg, 0.32 mmol, 1 eq) as the appropriate ketone and 3-bromoaniline (35 μL, 0.32 mmol, 1 eq) as the appropriate aniline, a racemic mixture of diatereoisomers was obtained. Purification by flash chromatography (5 g silica cartridge) eluting with a gradient of 0-10% EtOAc in DCM afforded a 5:2 mixture of diastereoisomers, isolated as an orange foam (73.2 mg, 0.15 mmol, 45%). The stereoisomers were separated by chiral chromatography. Column: Cellulose-1, 21×250 mm, 20 μm. Eluent: NH₄HCO₂ at pH4, MeCN-0.1% TFA 40:60. The stereoisomer pair eluting firstly was discarded. The stereoisomer eluting secondly was collected as Example 210, isolated as a brown gum (2.36 mg, 4.95 μmol, 16%). LRMS calculated for C₂₇H₂₆BrNO₂: 475; found: 476 (M+H). ¹H NMR (400 MHz, DMSO-d₆) δ ppm: 7.30-7.13 (m, 7H), 7.02-6.94 (m, 3H), 6.77-6.73 (m, 1H), 6.68-6.64 (m, 1H), 6.56-6.52 (m, 1H), 2.92-2.85 (m, 1H), 2.31-1.08 (m, 8H).

The stereoisomer eluting last was collected as Example 211, isolated as a brown gum (2.65 mg, 5.56 μmol, 18%). LRMS calculated for C₂₇H₂₆BrNO₂: 475; found: 476 (M+H). ¹H NMR (400 MHz, DMSO-d₆) δ ppm: 7.33-7.13 (m, 7H), 7.09-7.02 (m, 2H), 6.96 (t, J=8.0 Hz, 1H), 6.69-6.63 (m, 2H), 6.51-6.46 (m, 1H), 3.45-3.33 (m, 2H), 3.04-2.95 (m, 1H), 2.45-2.32 (m, 1H), 2.10-1.53 (m, 6H), 1.35-1.22 (m, 1H).

### Example 212 and Example 213 Example 212A 2-(1H-inden-2-yl)-4,4,5,5-tetramethyl-1,3,2-dioxaborolane

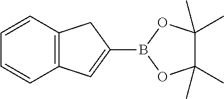 [SMILES: CC1(C)OB(C2=Cc3ccccc3C2)OC1(C)C]

To an oven-dried flask was added 2-bromo-1H-indene (10 g, 0.05 mol, 1 eq), TEA (21.4 mL, 0.15 mol, 3 eq) and 4,4,5,5-tetramethyl-1,3,2-dioxaborolane (11.2 mL, 0.08 mol, 1.5 eq) in 1,4-dioxane (100 mL). The mixture was sparged with N₂ followed by the addition of Pd(PPh₃)₂Cl₂ (1.08 g, 1.54 mmol, 0.03 eq) and the mixture was heated at 80° C. for 5.5 h. The mixture was allowed to cool to rt and partitioned between EtOAc and sat. aq. NaHCO₃ solution. The phases were separated, and the aq. phase was extracted with EtOAc. The combined organic extracts were washed with brine, dried (MgSO₄) and concentrated in vacuo. Purification by flash chromatography (330 g silica cartridge) eluting with a gradient of 0-8% EtOAc in heptane afforded Example 212A as a yellow solid (11 g, 45.4 mmol, 89%). ¹H NMR (400 MHz, DMSO-d₆) δ ppm: 7.54-7.48 (m, 3H), 7.32-7.22 (m, 2H), 3.48-3.44 (m, 2H), 1.28 (s, 12H).

### Example 212B 4-(1H-inden-2-yl)-1-methyl-1H-pyrazole

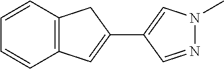

To an oven-dried microwave vial was added Example 212A (500 mg, 2.07 mmol, 1 eq), 4-bromo-1-methyl-1H-pyrazole (399 mg, 2.48 mmol, 1.2 eq) and Cs₂CO₃ (1.35 g, 4.13 mmol, 2 eq) in 1,4-dioxane (10 mL) and water (2.5 mL). The mixture was sparged with N₂ followed by the addition of Pd(dppf)Cl₂×DCM (169 mg, 0.21 mmol, 0.1 eq) and the mixture was heated at 120° C. for 1 h under microwave irradiation. The reaction was partitioned between EtOAc and sat. aq. NaHCO₃ solution, and the organic phase was washed with brine, dried (MgSO₄) and concentrated in vacuo. Purification by flash chromatography (25 g silica cartridge) eluting with a gradient of 0-5% EtOAc in DCM afforded Example 212B as an off-white solid (196 mg, 1 mmol, 48%). LRMS calculated for C₁₃H₁₂N₂: 196; found: 197 (M+H). ¹H NMR (400 MHz, DMSO-d₆) δ ppm: 7.99-7.97 (m, 1H), 7.78-7.75 (m, 1H), 7.44-7.39 (m, 1H), 7.33-7.28 (m, 1H), 7.24-7.18 (m, 1H), 7.09 (td, J=7.4, 1.2 Hz, 1H), 6.97-6.93 (m, 1H), 3.86 (s, 3H), 3.67-3.64 (m, 2H).

### Example 212C 4-(dispiro[[1,3]dioxolane-2,1′-cyclohexane-4′,1″-inden]-2″-yl)-1-methyl-1H-pyrazole

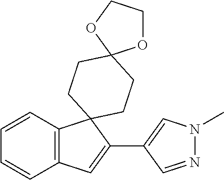 [SMILES: Cn1cc(C2=Cc3ccccc3C23CCC2(CC3)OCCO2)cn1]

Using General procedure 8a and Example 212B (148 mg, 0.76 mmol, 1 eq) as the appropriate indene, an intermediate was obtained which was purified by flash chromatography (5 g silica cartridge) eluting with a gradient of 0-6% EtOAc in DCM to obtain Example 212C as a brown solid (176 mg, 0.55 mmol, 72%). LRMS calculated for C₂₀H₂₂N₂O₂: 322; found: 323 (M+H). ¹H NMR (400 MHz, DMSO-d₆) δ ppm: 7.98-7.96 (m, 1H), 7.87-7.84 (m, 1H), 7.73-7.69 (m, 1H), 7.37-7.33 (m, 1H), 7.29-7.23 (m, 1H), 7.12 (td, J=7.5, 1.3 Hz, 1H), 6.97 (s, 1H), 4.08-3.97 (m, 4H), 3.90 (s, 3H), 2.44-2.33 (m, 2H), 2.23-2.12 (m, 2H), 1.88-1.79 (m, 2H), 1.17-1.09 (m, 2H).

### Example 212D 4-(2″,3″-dihydrodispiro[[1,3]dioxolane-2,1′-cyclohexane-4′,1″-inden]-2″-yl)-1-methyl-1H-pyrazole

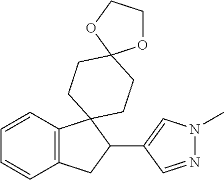 [SMILES: Cn1cc(C2Cc3ccccc3C23CCC2(CC3)OCCO2)cn1]

To a solution of Example 212C (176 mg, 0.55 mmol, 1 eq) in EtOH (4 mL) and THE (4 mL) was added 10% Pd/C (7 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1) and the combined filtrates were concentrated in vacuo to afford a racemate, Example 212D, isolated as a colourless glass (176 mg, 0.54 mmol, 99%). LRMS calculated for C₂₀H₂₄N₂O₂: 324; found: 325 (M+H). ¹H NMR (400 MHz, DMSO-d₆) δ ppm: 7.32-7.23 (m, 3H), 7.23-7.15 (m, 2H), 7.05-7.02 (m, 1H), 3.92-3.79 (m, 4H), 3.74 (s, 3H), 3.40 (dd, J=7.2, 5.4 Hz, 1H), 3.24 (dd, J=15.6, 7.2 Hz, 1H), 2.85 (dd, J=15.6, 5.4 Hz, 1H), 1.92-1.55 (m, 5H), 1.54-1.40 (m, 2H), 1.38-1.27 (m, 1H).

### Example 212E 2′-(1-methyl-1H-pyrazol-4-yl)-2′,3′-dihydrospiro[cyclohexane-1,1′-inden]-4-one

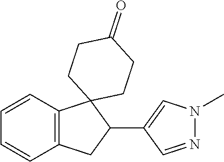 [SMILES: Cn1cc(C2Cc3ccccc3C23CCC(=O)CC3)cn1]

To a solution of Example 212D (176 mg, 0.54 mmol, 1 eq) in THE (4 mL) was added 2 M aq. HCl solution (1.63 mL, 3.25 mmol, 6 eq) and the mixture was heated at 45° C. for 4 h in a sealed flask. The mixture was allowed to cool to rt, partitioned between EtOAc and sat. aq. NaHCO₃ solution, and the organic phase was washed with brine, dried (MgSO₄) and concentrated in vacuo to afford a racemate, Example 212E, isolated as an orange gum (133 mg, 0.47 mmol, 85%) that was used directly in the subsequent step without further purification. LRMS calculated for C₁₈H₂₀N₂O: 280; found: 281 (M+H). ¹H NMR (400 MHz, DMSO-d₆) δ ppm: 7.42-7.36 (m, 2H), 7.33-7.26 (m, 1H), 7.26-7.18 (m, 2H), 7.18-7.15 (m, 1H), 3.75 (s, 3H), 3.58 (dd, J=7.2, 5.9 Hz, 1H), 3.30 (dd, J=15.7, 7.2 Hz, 1H), 2.95 (dd, J=15.7, 5.9 Hz, 1H), 2.67-2.47 (m, 2H), 2.21-2.09 (m, 2H), 2.08-1.98 (m, 1H), 1.95-1.84 (m, 2H), 1.58-1.49 (m, 1H).

### Example 212 (1r,4r)-4-(3-bromoanilino)-2′-(1-methyl-1H-pyrazol-4-yl)-2′,3′-dihydrospiro[cyclohexane-1,1′-indene]-4-carboxylic acid

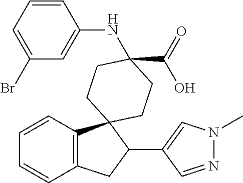

and

### Example 213 (1s,4s)-4-(3-bromoanilino)-2′-(1-methyl-1H-pyrazol-4-yl)-2′,3′-dihydrospiro[cyclohexane-1,1′-indene]-4-carboxylic acid

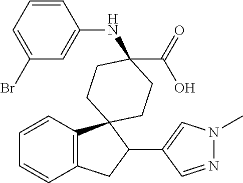

Using General Procedure 10 and Example 212E (133 mg, 0.47 mmol, 1 eq) as the appropriate ketone and 3-bromoaniline (0.06 mL, 0.56 mmol, 1.2 eq) as the appropriate aniline, a racemic mixture of diastereoisomers was obtained. The diastereoisomers were separated by flash chromatography (5 g silica cartridge) eluting with a gradient of 0-5% MeOH in DCM. The racemic diastereoisomer eluting earlier was collected as Example 212, isolated as a yellow foam (67 mg, 0.14 mmol, 27%). LRMS calculated for C₂₅H₂₆BrN₃O₂: 479; found: 480 (M+H). ¹H NMR (400 MHz, DMSO-d₆) δ ppm: 12.65 (br s, 1H), 7.43-7.36 (m, 2H), 7.31-7.14 (m, 4H), 6.97 (t, J=8.1 Hz, 1H), 6.70-6.63 (m, 2H), 6.49-6.43 (m, 1H), 6.04 (br s, 1H), 3.77 (s, 3H), 3.26-3.10 (m, 2H), 2.95 (dd, J=15.4, 7.8 Hz, 1H), 2.45-2.35 (m, 1H), 2.07-1.93 (m, 3H), 1.86-1.75 (m, 1H), 1.66-1.56 (m, 1H), 1.37-1.21 (m, 2H).

The racemic diastereoisomer eluting later was collected as Example 213, isolated as a brown solid (27.6 mg, 0.06 mmol, 11%). LRMS calculated for C₂₅H₂₆BrN₃O₂: 479; found: 480 (M+H). ¹H NMR (400 MHz, DMSO-d₆) δ ppm: 7.29-7.14 (m, 5H), 7.02-6.92 (m, 2H), 6.79-6.76 (m, 1H), 6.68-6.63 (m, 1H), 6.60-6.55 (m, 1H), 3.72 (s, 3H), 3.48 (dd, J=7.2, 3.7 Hz, 1H), 3.36-3.28 (m, 1H), 2.83-2.75 (m, 1H), 2.25-2.14 (m, 1H), 2.10-2.02 (m, 1H), 1.92-1.58 (m, 5H), 1.26-1.17 (m, 1H).

### Example 214

### Example 214A 2-(2-methylphenyl)-1H-indene

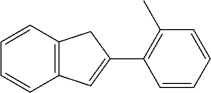 [SMILES: Cc1ccccc1C1=Cc2ccccc2C1]

To an oven-dried microwave vial was added Example 212A (500 mg, 2.07 mmol, 1 eq), 1-iodo-2-methylbenzene (0.29 mL, 2.28 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The mixture was concentrated in vacuo, partitioned between DCM and sat. aq. NaHCO₃ solution, and the organic phase was washed with brine, dried (MgSO₄) and concentrated in vacuo. Purification by flash chromatography (20 g silica cartridge) eluting with a gradient of 0-5% DCM in heptane afforded Example 214A as a white solid (332 mg, 1.61 mmol, 78%). ¹H NMR (400 MHz, DMSO-d₆) δ ppm: 7.53-7.48 (m, 2H), 7.47-7.43 (m, 1H), 7.31-7.17 (m, 5H), 7.10-7.07 (m, 1H), 3.89-3.84 (m, 2H), 2.48 (s, 3H).

### Example 214B 2″-(2-methylphenyl)dispiro[[1,3]dioxolane-2,1′-cyclohexane-4′,1″-indene]

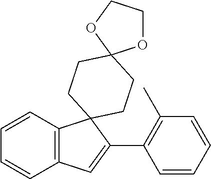 [SMILES: Cc1ccccc1C1=Cc2ccccc2C12CCC1(CC2)OCCO1]

Using General procedure 8a and Example 214A (166 mg, 0.8 mmol, 1 eq) as the appropriate indene afforded Example 214B as an orange foam (222 mg, 0.67 mmol, 83%). ¹H NMR (400 MHz, DMSO-d₆) δ ppm: 7.70-7.64 (m, 1H), 7.41-7.36 (m, 1H), 7.34-7.17 (m, 5H), 7.12-7.07 (m, 1H), 6.56 (s, 1H), 3.91-3.85 (m, 2H), 3.82-3.77 (m, 2H), 2.19 (s, 3H), 2.12-2.01 (m, 2H), 2.01-1.90 (m, 2H), 1.76-1.67 (m, 2H), 1.52-1.42 (m, 2H).

### Example 214C 2″-(2-methylphenyl)-2″,3″-dihydrodispiro[[1,3]dioxolane-2,1′-cyclohexane-4′,1″-indene]

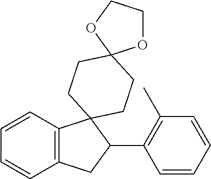 [SMILES: Cc1ccccc1C1Cc2ccccc2C12CCC1(CC2)OCCO1]

To a solution of Example 214B (222 mg, 0.67 mmol, 1 eq) in EtOH (3 mL) and THE (3 mL) was added 10% Pd/C (7 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1). The combined filtrates were concentrated in vacuo to afford a racemate, Example 214C, isolated as a white solid (217 mg, 0.65 mmol, 97%). ¹H NMR (400 MHz, DMSO-d₆) δ ppm: 7.30-7.12 (m, 5H), 7.05 (td, J=7.4, 1.4 Hz, 1H), 6.99-6.92 (m, 1H), 6.75-6.70 (m, 1H), 3.90-3.75 (m, 5H), 3.48 (dd, J=16.2, 8.1 Hz, 1H), 2.81 (dd, J=16.2, 3.2 Hz, 1H), 2.41 (s, 3H), 1.91-1.71 (m, 4H), 1.70-1.60 (m, 1H), 1.53-1.44 (m, 1H), 1.44-1.34 (m, 1H), 1.17-1.07 (m, 1H).

### Example 214D 2′-(2-methylphenyl)-2′,3′-dihydrospiro[cyclohexane-1,1′-inden]-4-one

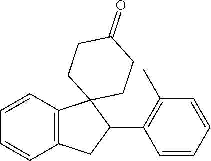 [SMILES: Cc1ccccc1C1Cc2ccccc2C12CCC(=O)CC2]

To a solution of Example 214C (217 mg, 0.65 mmol, 1 eq) in THE (4 mL) was added 2 M aq. HCl solution (1.95 mL, 3.89 mmol, 6 eq) and the mixture was heated at 55° C. for 16 h in a sealed flask. The mixture was allowed to cool to rt, concentrated in vacuo, and partitioned between EtOAc and sat. aq. NaHCO₃ solution. The organic phase was washed with brine, dried (MgSO₄) and concentrated in vacuo to afford a racemate, Example 214D, isolated as a white solid (172 mg, 0.59 mmol, 91%) that was used directly in the subsequent step without further purification. ¹H NMR (400 MHz, DMSO-d₆) δ ppm: 7.44-7.38 (m, 1H), 7.33-7.20 (m, 3H), 7.20-7.15 (m, 1H), 7.08 (td, J=7.4, 1.4 Hz, 1H), 7.01-6.95 (m, 1H), 6.86-6.80 (m, 1H), 3.95 (dd, J=8.0, 3.8 Hz, 1H), 3.53 (dd, J=16.3, 8.0 Hz, 1H), 2.92 (dd, J=16.3, 3.8 Hz, 1H), 2.66-2.38 (m, 5H), 2.17-2.00 (m, 3H), 1.98-1.83 (m, 2H), 1.77-1.67 (m, 1H).

### Example 214 4-(3-bromoanilino)-2′-(2-methylphenyl)-2′,3′-dihydrospiro[cyclohexane-1,1′-indene]-4-carboxylic acid

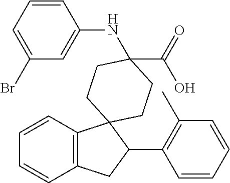 [SMILES: Cc1ccccc1C1Cc2ccccc2C12CCC(Nc1cccc(Br)c1)(C(=O)O)CC2]

Using General Procedure 10 and Example 214D (172 mg, 0.59 mmol, 1 eq) as the appropriate ketone and 3-bromoaniline (0.06 mL, 0.56 mmol, 1 eq) as the appropriate aniline, a racemic mixture of diastereoisomers was obtained. Purification by flash chromatography (5 g silica cartridge) eluting with a gradient 0-10% EtOAc in DCM, followed by preparative HPLC at pH 4 eluting with a gradient of 68-78% MeCN in water afforded a racemic mixture of diastereoisomers, Example 214, isolated as an off-white foam (45.3 mg, 0.09 mmol, 16%). LRMS calculated for C₂₈H₂₈BrNO₂: 489; found: 490 (M+H). ¹H NMR (400 MHz, DMSO-d₆) δ ppm: 12.62 (br s, 1H), 7.33-7.12 (m, 5H), 7.08-6.88 (m, 3H), 6.76-6.49 (m, 4H), 6.22 (br s, 1H), 3.87-3.81/3.75-3.69 (m, 1H), 3.61-3.52/3.52-3.42 (m, 1H), 2.87-2.79/2.77-2.69 (m, 1H), 2.49-1.30 (m, 11H).

### Example 215

### Example 215A 2-(3-methylphenyl)-1H-indene

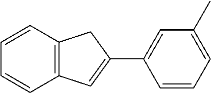 [SMILES: Cc1cccc(C2=Cc3ccccc3C2)c1]

To an oven-dried microwave vial was added Example 212A (500 mg, 2.07 mmol, 1 eq), 1-iodo-3-methylbenzene (0.29 mL, 2.28 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The mixture was concentrated in vacuo, partitioned between DCM and sat. aq. NaHCO₃ solution. The organic phase was washed with brine, dried (MgSO₄) and concentrated in vacuo. Purification by flash chromatography (20 g silica cartridge) eluting with a gradient of 0-5% DCM in heptane afforded Example 215A as a white solid (166 mg, 0.80 mmol, 39%). ¹H NMR (400 MHz, DMSO-d₆) δ ppm: 7.57-7.46 (m, 3H), 7.45-7.36 (m, 2H), 7.33-7.23 (m, 2H), 7.20-7.15 (m, 1H), 7.14-7.09 (m, 1H), 3.85-3.82 (m, 2H), 2.36 (s, 3H).

### Example 215B 2″-(3-methylphenyl)dispiro[[1,3]dioxolane-2,1′-cyclohexane-4′,1″-indene]

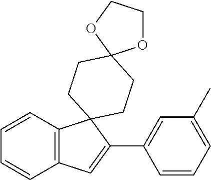 [SMILES: Cc1cccc(C2=Cc3ccccc3C23CCC2(CC3)OCCO2)c1]

Using General procedure 8a and Example 215A (166 mg, 0.8 mmol, 1 eq) as the appropriate indene afforded Example 215B, isolated as an orange gum (213 mg, 0.64 mmol, 80%). ¹H NMR (400 MHz, DMSO-d₆) δ ppm: 7.76-7.70 (m, 1H), 7.46-7.36 (m, 3H), 7.35-7.26 (m, 2H), 7.23-7.15 (m, 2H), 7.03 (s, 1H), 3.95 (s, 4H), 2.44-2.31 (m, 5H), 2.20-2.10 (m, 2H), 1.85-1.76 (m, 2H), 1.31-1.21 (m, 2H).

### Example 215C 2″-(3-methylphenyl)-2″,3″-dihydrodispiro[[1,3]dioxolane-2,1′-cyclohexane-4′,1″-indene]

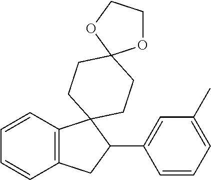 [SMILES: Cc1cccc(C2Cc3ccccc3C23CCC2(CC3)OCCO2)c1]

To a solution of Example 215B (213 mg, 0.64 mmol, 1 eq) in EtOH (3 mL) and THE (3 mL) was added 10% Pd/C (7 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1). The combined filtrates were concentrated in vacuo to afford a racemate, Example 215C, isolated as a white solid (202 mg, 0.60 mmol, 94%). ¹H NMR (400 MHz, DMSO-d₆) δ ppm: 7.30-7.16 (m, 4H), 7.07 (t, J 7.6 Hz, 1H), 7.01-6.96 (m, 1H), 6.92-6.87 (m, 1H), 6.81-6.74 (m, 1H), 3.89-3.77 (m, 4H), 3.48 (dd, J=7.7, 3.5 Hz, 1H), 3.40 (dd, J=15.9, 7.7 Hz, 1H), 2.92 (dd, J=15.9, 3.5 Hz, 1H), 2.21 (s, 3H), 1.95-1.71 (m, 4H), 1.67-1.56 (m, 1H), 1.43-1.23 (m, 3H).

### Example 215D 2′-(3-methylphenyl)-2′,3′-dihydrospiro[cyclohexane-1,1′-inden]-4-one

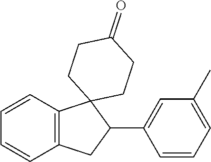 [SMILES: Cc1cccc(C2Cc3ccccc3C23CCC(=O)CC3)c1]

To a solution of Example 215C (202 mg, 0.60 mmol, 1 eq) in THE (4 mL) was added 2 M aq. HCl solution (1.95 mL, 3.89 mmol, 6 eq) and the mixture was heated at 55° C. for 16 h in a sealed flask. The mixture was allowed to cool to rt, concentrated in vacuo, and partitioned between EtOAc and sat. aq. NaHCO₃ solution. The organic phase was washed with brine, dried (MgSO₄) and concentrated in vacuo to afford a racemate, Example 215D, isolated as a pale yellow gum (171 mg, 0.59 mmol, 98%) that was used directly in the subsequent step without further purification. ¹H NMR (400 MHz, DMSO-d₆) δ ppm: 7.40-7.28 (m, 2H), 7.28-7.19 (m, 2H), 7.10 (t, J=7.5 Hz, 1H), 7.05-6.98 (m, 2H), 6.92-6.88 (m, 1H), 3.69 (dd, J=7.7, 4.2 Hz, 1H), 3.46 (dd, J=16.2, 7.7 Hz, 1H), 3.03 (dd, J=16.2, 4.2 Hz, 1H), 2.80-2.70 (m, 1H), 2.47-2.37 (m, 1H), 2.22 (s, 3H), 2.16-1.98 (m, 4H), 1.93-1.83 (m, 1H), 1.56-1.47 (m, 1H).

### Example 215 4-(3-bromoanilino)-2′-(3-methylphenyl)-2′,3′-dihydrospiro[cyclohexane-1,1′-indene]-4-carboxylic acid

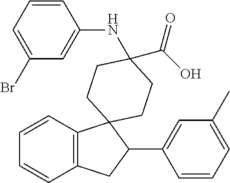 [SMILES: Cc1cccc(C2Cc3ccccc3C23CCC(Nc2cccc(Br)c2)(C(=O)O)CC3)c1]

Using General Procedure 10 and Example 215D (171 mg, 0.59 mmol, 1 eq) as the appropriate ketone and 3-bromoaniline (0.06 mL, 0.56 mmol, 1 eq) as the appropriate aniline, a racemic mixture of diastereoisomers was obtained. Purification by flash chromatography (5 g silica cartridge) eluting with a gradient of 0-10% EtOAc in DCM, followed by purification by preparative HPLC at pH 4 eluting with a gradient of 68-78% MeCN in water afforded a racemic mixture of diastereoisomers, Example 215, isolated as an off-white glass (48.5 mg, 0.10 mmol, 17%). LRMS calculated for C₂₈H₂₈BrNO₂: 489; found: 490 (M+H). ¹H NMR (400 MHz, DMSO-d₆) δ ppm: 12.59 (br s, 1H), 7.34-7.17 (m, 4H), 7.12-7.05 (m, 1H), 7.03-6.79 (m, 4H), 6.77-6.63 (m, 2H), 6.57-6.53/6.50-6.45 (m, 1H), 6.19 (br s, 1H), 3.55-3.29 (m, 2H), 3.04-2.84 (m, 1H), 2.43-2.17 (m, 4H), 2.13-1.51 (m, 6H), 1.38-1.13 (m, 1H).

### Example 216

### Example 216A 2-(4-methylphenyl)-1H-indene

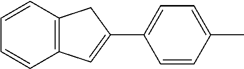 [SMILES: Cc1ccc(C2=Cc3ccccc3C2)cc1]

To an oven-dried microwave vial was added Example 212A (500 mg, 2.07 mmol, 1 eq), 1-iodo-4-methylbenzene (497 mg, 2.28 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The mixture was concentrated in vacuo and partitioned between DCM and sat. aq. NaHCO₃ solution. The organic phase was washed with brine, dried (MgSO₄) and concentrated in vacuo. Purification by flash chromatography (20 g silica cartridge) eluting with a gradient of 0-5% DCM in heptane afforded Example 216A as a white solid (259 mg, 1.26 mmol, 61%). ¹H NMR (400 MHz, DMSO-d₆) δ ppm: 7.64-7.58 (m, 2H), 7.50-7.45 (m, 1H), 7.43-7.38 (m, 1H), 7.35-7.32 (m, 1H), 7.28-7.20 (m, 3H), 7.19-7.13 (m, 1H), 3.84-3.80 (m, 2H), 2.33 (s, 3H).

### Example 216B 2″-(4-methylphenyl)dispiro[[1,3]dioxolane-2,1′-cyclohexane-4′,1″-indene]

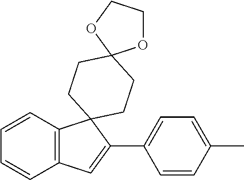 [SMILES: Cc1ccc(C2=Cc3ccccc3C23CCC2(CC3)OCCO2)cc1]

Using General procedure 8a an Example 216A (166 mg, 0.8 mmol, 1 eq) as the appropriate indene, Example 216B was isolated as an orange gum (200 mg, 0.60 mmol, 75%). ¹H NMR (400 MHz, DMSO-d₆) δ ppm: 7.72 (d, J=7.4 Hz, 1H), 7.52-7.47 (m, 2H), 7.43-7.39 (m, 1H), 7.31-7.22 (m, 3H), 7.18 (td, J=7.5, 1.3 Hz, 1H), 7.01 (s, 1H), 3.99-3.92 (m, 4H), 2.42-2.31 (m, 5H), 2.21-2.10 (m, 2H), 1.84-1.76 (m, 2H), 1.29-1.21 (m, 2H).

### Example 216C 2″-(4-methylphenyl)-2″,3″-dihydrodispiro[[1,3]dioxolane-2,1′-cyclohexane-4′,1″-indene]

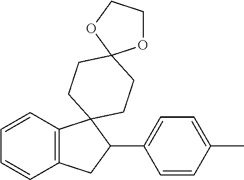 [SMILES: Cc1ccc(C2Cc3ccccc3C23CCC2(CC3)OCCO2)cc1]

To a solution of Example 216B (200 mg, 0.60 mmol, 1 eq) in EtOH (3 mL) and THE (3 mL) was added 10% Pd/C (7 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1). The combined filtrates were concentrated in vacuo to afford a racemate, Example 216C, isolated as a white solid (190 mg, 0.57 mmol, 94%). ¹H NMR (400 MHz, DMSO-d₆) δ ppm: 7.30-7.16 (m, 4H), 7.03-6.98 (m, 2H), 6.95-6.89 (m, 2H), 3.89-3.77 (m, 4H), 3.51-3.46 (m, 1H), 3.44-3.35 (m, 1H), 2.91 (dd, J=15.9, 3.6 Hz, 1H), 2.23 (s, 3H), 1.96-1.70 (m, 4H), 1.67-1.55 (m, 1H), 1.43-1.22 (m, 3H).

### Example 216D 2′-(4-methylphenyl)-2′,3′-dihydrospiro[cyclohexane-1,1′-inden]-4-one

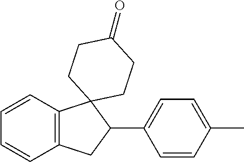 [SMILES: Cc1ccc(C2Cc3ccccc3C23CCC(=O)CC3)cc1]

To a solution of Example 216C (190 mg, 0.57 mmol, 1 eq) in THE (4 mL) was added 2 M aq. HCl solution (1.95 mL, 3.89 mmol, 6.8 eq) and the mixture was heated at 55° C. for 16 h in a sealed flask. The mixture was allowed to cool to rt, concentrated in vacuo and partitioned between EtOAc and sat. aq. NaHCO₃ solution. The organic phase was washed with brine, dried (MgSO₄) and concentrated in vacuo to afford a racemate, Example 216D, isolated as a pale yellow gum (156 mg, 0.54 mmol, 95%) that was used directly in the subsequent step without further purification. ¹H NMR (400 MHz, DMSO-d₆) δ ppm: 7.39-7.28 (m, 2H), 7.27-7.19 (m, 2H), 7.07-7.00 (m, 4H), 3.70 (dd, J=7.7, 4.1 Hz, 1H), 3.47 (dd, J=16.1, 7.7 Hz, 1H), 3.01 (dd, J=16.1, 4.1 Hz, 1H), 2.82-2.70 (m, 1H), 2.46-2.36 (m, 1H), 2.24 (s, 3H), 2.13-1.99 (m, 4H), 1.92-1.82 (m, 1H), 1.54-1.45 (m, 1H).

### Example 216 4-(3-bromoanilino)-2′-(4-methylphenyl)-2′,3′-dihydrospiro[cyclohexane-1,1′-indene]-4-carboxylic acid

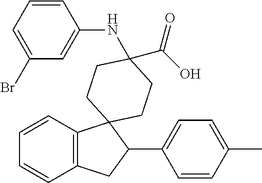 [SMILES: Cc1ccc(C2Cc3ccccc3C23CCC(Nc2cccc(Br)c2)(C(=O)O)CC3)cc1]

Using General Procedure 10 and Example 216D (156 mg, 0.54 mmol) as the appropriate ketone and 3-bromoaniline (0.06 mL, 0.59 mmol, 1.09 eq) as the appropriate aniline, a racemic mixture of diastereoisomers was obtained. Purification by flash chromatography (5 g silica cartridge) eluting with a gradient of 0-10% EtOAc in DCM, followed by preparative HPLC at pH 4 eluting with a gradient of 70-80% MeCN in water afforded a racemic mixture of diastereoisomers, Example 216, isolated as an off-white foam (62.8 mg, 0.13 mmol, 24%). LRMS calculated for C₂₈H₂₈BrNO₂: 489; found: 490 (M+H). ¹H NMR (400 MHz, DMSO-d₆) δ ppm: 7.32-7.16 (m, 4H), 7.05-6.81 (m, 5H), 6.78-6.62 (m, 2H), 6.57-6.52/6.51-6.46 (m, 1H), 6.20 (br s, 1H), 3.56-3.32 (m, 2H), 3.00-2.81 (m, 1H), 2.43-2.16 (m, 4H), 2.13-1.51 (m, 6H), 1.35-1.09 (m, 1H).

### Example 217 and Example 218

### Example 217A 2-(4-methoxyphenyl)-1H-indene

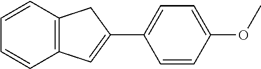 [SMILES: COc1ccc(C2=Cc3ccccc3C2)cc1]

To an oven-dried microwave vial was added Example 212A (500 mg, 2.07 mmol, 1 eq), 4-bromoanisole (284 μL, 2.27 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The reaction was partitioned between EtOAc and sat aq. NaHCO₃ solution, and the organic phase was washed with brine, dried (MgSO₄) and concentrated in vacuo. Purification by flash chromatography (25 g silica cartridge) eluting with a gradient of 0-17% DCM in heptane afforded Example 217A as brown crystals (168 mg, 0.76 mmol, 37%). ¹H NMR (400 MHz, DMSO-d₆) δ ppm: 7.69-7.63 (m, 2H), 7.48-7.43 (m, 1H), 7.40-7.35 (m, 1H), 7.27-7.21 (m, 2H), 7.14 (td, J=7.4, 1.2 Hz, 1H), 7.01-6.95 (m, 2H), 3.83-3.77 (s, 5H).

### Example 217B 2″-(4-methoxyphenyl)dispiro[[1,3]dioxolane-2,1′-cyclohexane-4′,1″-indene]

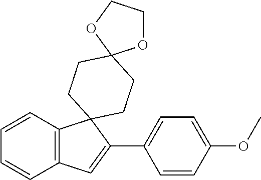 [SMILES: COc1ccc(C2=Cc3ccccc3C23CCC2(CC3)OCCO2)cc1]

Using General procedure 8a and Example 217A (168 mg, 0.76 mmol, 1 eq) as the appropriate indene and purification by flash chromatography (5 g silica cartridge) eluting with a gradient of 0-50% DCM in heptane, Example 217B was obtained as a brown solid (203 mg, 0.58 mmol, 77%). ¹H NMR (400 MHz, DMSO-d₆) δ ppm: 7.74-7.69 (m, 1H), 7.62-7.56 (m, 2H), 7.42-7.38 (m, 1H), 7.31-7.25 (m, 1H), 7.17 (td, J=7.5, 1.3 Hz, 1H), 7.03-6.98 (m, 3H), 4.00-3.94 (m, 4H), 3.81 (s, 3H), 2.40 (td, J=13.7, 4.5 Hz, 2H), 2.16 (td, J=13.6, 4.6 Hz, 2H), 1.86-1.77 (m, 2H), 1.28-1.19 (m, 2H).

### Example 217C 2″-(4-methoxyphenyl)-2″,3″-dihydrodispiro[[1,3]dioxolane-2,1′-cyclohexane-4′,1″-indene]

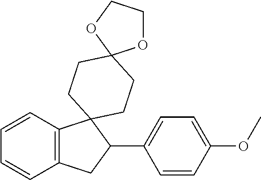 [SMILES: COc1ccc(C2Cc3ccccc3C23CCC2(CC3)OCCO2)cc1]

To a solution of Example 217B (203 mg, 0.58 mmol, 1 eq) in EtOH (4 mL) and THE (4 mL) was added 10% Pd/C (7 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1). The combined filtrates were concentrated in vacuo to afford a racemate, Example 217C, isolated as a white solid (203 mg, 0.58 mmol, 100%). ¹H NMR (400 MHz, DMSO-d₆) δ ppm: 7.30-7.16 (m, 4H), 6.99-6.91 (m, 2H), 6.80-6.73 (m, 2H), 3.90-3.77 (m, 4H), 3.70 (s, 3H), 3.50-3.45 (m, 1H), 3.43-3.33 (m, 1H), 2.90 (dd, J=15.9, 3.8 Hz, 1H), 1.94-1.70 (m, 4H), 1.66-1.55 (m, 1H), 1.45-1.22 (m, 3H).

### Example 217D 2′-(4-methoxyphenyl)-2′,3′-dihydrospiro[cyclohexane-1,1′-inden]-4-one

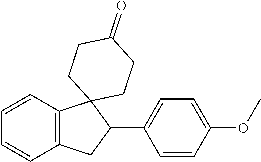 [SMILES: COc1ccc(C2Cc3ccccc3C23CCC(=O)CC3)cc1]

To a solution of Example 217C (203 mg, 0.58 mmol, 1 eq) in THE (4 mL) was added 2 M aq. HCl solution (1.63 mL, 3.25 mmol, 5.6 eq) and the mixture was heated at 55° C. for 16 h in a sealed flask. The mixture was allowed to cool to rt, concentrated in vacuo and partitioned between EtOAc and sat. aq. NaHCO₃ solution. The combined organic extracts were washed with brine, dried (MgSO₄) and concentrated in vacuo to afford a racemate, Example 217D, isolated as a pale yellow gum (171 mg, 0.56 mmol, 90%) that was used directly in the subsequent step without further purification. ¹H NMR (400 MHz, DMSO-d₆) δ ppm: 7.39-7.18 (m, 4H), 7.11-7.05 (m, 2H), 6.82-6.76 (m, 2H), 3.72-3.66 (m, 4H), 3.45 (dd, J=16.1, 7.6 Hz, 1H), 3.00 (dd, J=16.1, 4.2 Hz, 1H), 2.80-2.69 (m, 1H), 2.47-2.37 (m, 1H), 2.15-1.98 (m, 4H), 1.92-1.82 (m, 1H), 1.55-1.45 (m, 1H).

### Example 217 (1r,2′S,4S)-4-(3-bromoanilino)-2′-(4-methoxyphenyl)-2′,3′-dihydrospiro[cyclohexane-1,1′-indene]-4-carboxylic acid

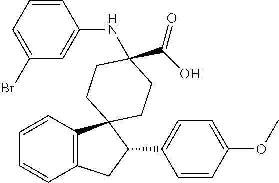 [SMILES: COc1ccc([C@@H]2Cc3ccccc3[C@]23CC[C@@](Nc2cccc(Br)c2)(C(=O)O)CC3)cc1]

### Example 218 (1r,2′R,4R)-4-(3-bromoanilino)-2′-(4-methoxyphenyl)-2′,3′-dihydrospiro[cyclohexane-1,1′-indene]-4-carboxylic acid

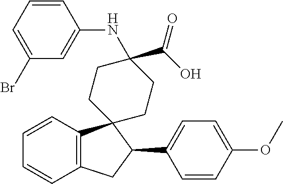 [SMILES: COc1ccc([C@H]2Cc3ccccc3[C@]23CC[C@@](Nc2cccc(Br)c2)(C(=O)O)CC3)cc1]

Using General Procedure 10 and Example 217D (171 mg, 0.56 mmol, 1 eq) as the appropriate ketone and 3-bromoaniline (0.06 mL, 0.56 mmol, 1 eq) as the appropriate aniline, a racemic mixture of diastereoisomers was obtained. Purification by flash chromatography (5 g silica cartridge) eluting with a gradient of 0-10% EtOAc in DCM afforded the major isomer as a racemic single diastereoisomer, isolated as an orange foam (43 mg, 0.08 mmol, 15%). The enantiomers were separated by chiral HPLC. Column: AD, 50×500 mm, 20 m. Eluents: 50:50 EtOH/Heptane+0.05% HCOOH. The enantiomer eluting earlier was collected as Example 217. ¹H NMR (500 MHz, DMSO-d₆) δ ppm: 7.31-7.14 (m, 4H), 6.95 (dm, 2H), 6.94 (t, 1H), 6.76 (dm, 2H), 6.68 (t, 1H), 6.64 (d, 1H), 6.48 (dd, 1H), 3.69 (s, 3H), 3.35 (m, 1H), 3.34/2.94 (m+m, 2H), 2.36/2.03/1.92/1.59 (m+m/m+m, 4H), 1.82/1.59/1.59/1.27 (m+m/m+m, 4H). HRMS calculated for C₂₈H₂₈NO₃Br: 505.1253; found: 506.1308 (M+H).

The enantiomer eluting later was collected as Example 218. HRMS calculated for C₂₈H₂₈NO₃Br: 505.1253; found: 506.1305 (M+H).

### Example 219 and Example 220

### Example 219A 2-(3-methoxyphenyl)-1H-indene

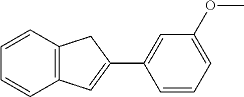 [SMILES: COc1cccc(C2=Cc3ccccc3C2)c1]

To an oven-dried microwave vial was added Example 212A (500 mg, 2.07 mmol, 1 eq), 1-bromo-3-methoxybenzene (285 μL, 2.27 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The reaction was partitioned between EtOAc and sat. aq. NaHCO₃ solution, and the organic phase was washed with brine, dried (MgSO₄) and concentrated in vacuo. Purification by flash chromatography (25 g silica cartridge) eluting with a gradient of 0-15% EtOAc in heptane afforded Example 219A as an off-white solid (233 mg, 1.05 mmol, 51%). ¹H NMR (400 MHz, DMSO-d₆) δ ppm: 7.51-7.47 (m, 1H), 7.45-7.40 (m, 2H), 7.35-7.24 (m, 4H), 7.18 (td, J=7.4, 1.2 Hz, 1H), 6.90-6.86 (m, 1H), 3.86-3.84 (m, 2H), 3.82 (s, 3H).

### Example 219B 2″-(3-methoxyphenyl)dispiro[[1,3]dioxolane-2,1′-cyclohexane-4′,1″-indene]

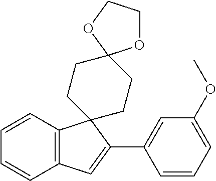 [SMILES: COc1cccc(C2=Cc3ccccc3C23CCC2(CC3)OCCO2)c1]

Using General procedure 8a and Example 219A (168 mg, 0.76 mmol, 1 eq) as the appropriate indene and then purification by flash chromatography (5 g silica cartridge) eluting with a gradient of 0-50% DCM in heptane, Example 219B was isolated as a yellow gum (216 mg, 0.62 mmol, 82%). ¹H NMR (400 MHz, DMSO-d₆) δ ppm: 7.77-7.71 (m, 1H), 7.46-7.41 (m, 1H), 7.38-7.27 (m, 2H), 7.26-7.17 (m, 3H), 7.15-7.12 (m, 1H), 6.95-6.90 (m, 1H), 4.00-3.92 (m, 4H), 3.82 (s, 3H), 2.44 (td, J=13.6, 4.5 Hz, 2H), 2.16 (td, J=13.6, 4.7 Hz, 2H), 1.86-1.77 (m, 2H), 1.30-1.21 (m, 2H).

### Example 219C 2″-(3-methoxyphenyl)-2″,3″-dihydrodispiro[[1,3]dioxolane-2,1′-cyclohexane-4′,1″-indene]

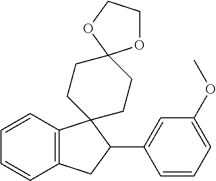 [SMILES: COc1cccc(C2Cc3ccccc3C23CCC2(CC3)OCCO2)c1]

To a solution of Example 219B (216 mg, 0.62 mmol, 1 eq) in EtOH (4 mL) and THE (4 mL) was added 10% Pd/C (7 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1). The combined filtrates were concentrated in vacuo to afford a racemate, Example 219C, isolated as a white solid (209 mg, 0.60 mmol, 96%). ¹H NMR (400 MHz, DMSO-d₆) δ ppm: 7.30-7.17 (m, 4H), 7.11 (t, J 7.9 Hz, 1H), 6.78-6.72 (m, 1H), 6.64-6.54 (m, 2H), 3.90-3.77 (m, 4H), 3.64 (s, 3H), 3.52 (dd, J=7.6, 3.3 Hz, 1H), 3.46-3.37 (m, 1H), 2.91 (dd, J=16.2, 3.3 Hz, 1H), 1.97-1.72 (m, 4H), 1.66-1.54 (m, 1H), 1.45-1.26 (m, 3H).

### Example 219D 2′-(3-methoxyphenyl)-2′,3′-dihydrospiro[cyclohexane-1,1′-inden]-4-one

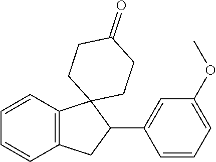 [SMILES: COc1cccc(C2Cc3ccccc3C23CCC(=O)CC3)c1]

To a solution of Example 219C (209 mg, 0.62 mmol, 1 eq) in THE (4 mL) was added 2 M aq. HCl solution (1.63 mL, 3.25 mmol, 5.2 eq) and the mixture was heated at 55° C. for 16 h in a sealed flask. The mixture was allowed to cool to rt, concentrated in vacuo and partitioned between EtOAc and sat. aq. NaHCO₃ solution. The organic phase was washed with brine, dried (MgSO₄) and concentrated in vacuo to afford a racemate, Example 219D, isolated as a pale yellow gum (143 mg, 0.47 mmol, 77%) that was used directly in the subsequent step without further purification. ¹H NMR (400 MHz, DMSO-d₆) δ ppm: 7.38-7.18 (m, 4H), 7.13 (t, J=7.1 Hz, 1H), 6.79-6.72 (m, 2H), 6.71-6.67 (m, 1H), 3.73 (dd, J=7.5, 3.5 Hz, 1H), 3.66 (s, 3H), 3.49 (dd, J=16.1, 7.5 Hz, 1H), 3.01 (dd, J=16.1, 3.5 Hz, 1H), 2.85-2.74 (m, 1H), 2.43-2.34 (m, 1H), 2.23-2.00 (m, 4H), 1.91-1.90 (m, 1H), 1.59-1.49 (m, 1H).

### Example 219 (1r,2′S,4S)-4-(3-bromoanilino)-2′-(3-methoxyphenyl)-2′,3′-dihydrospiro[cyclohexane-1,1′-indene]-4-carboxylic acid

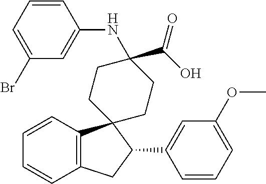 [SMILES: COc1cccc([C@@H]2Cc3ccccc3[C@]23CC[C@@](Nc2cccc(Br)c2)(C(=O)O)CC3)c1]

and

### Example 220 (1r,2′R,4R)-4-(3-bromoanilino)-2′-(3-methoxyphenyl)-2′,3′-dihydrospiro[cyclohexane-1,1′-indene]-4-carboxylic acid

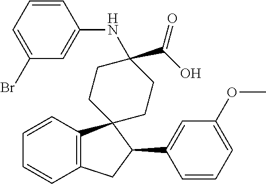 [SMILES: COc1cccc([C@H]2Cc3ccccc3[C@]23CC[C@@](Nc2cccc(Br)c2)(C(=O)O)CC3)c1]

Using General Procedure 10 and Example 219D (143 mg, 0.47 mmol) as the appropriate ketone and 3-bromoaniline (0.06 mL, 0.56 mmol, 1.2 eq) as the appropriate aniline, a racemic mixture of diastereoisomers was obtained. Purification by flash chromatography (5 g silica cartridge) eluting with a gradient of 0-10% EtOAc in DCM afforded the major isomer as a racemic single diastereoisomer, isolated as an orange foam (24.9 mg, 0.02 mmol, 10%). The enantiomers were separated by chiral HPLC. Column: AD, 50×500 mm, 20 m. Eluents: 30:70 EtOH/Heptane+0.05% HCOOH. The enantiomer eluting earlier was collected as Example 219. ¹H NMR (500 MHz, DMSO-d₆) δ ppm: 7.32-7.17 (m, 4H), 7.12 (t, 1H), 6.95 (t, 1H), 6.75 (dd, 1H), 6.68 (t, 1H), 6.65 (dd, 1H), 6.60 (br d, 1H), 6.58 (br s, 1H), 6.49 (dd, 1H), 3.65 (s, 3H), 3.38 (m, 1H), 3.37/2.96 (m+m, 2H), 2.36/2.04/1.94/1.60 (m+m/m+m, 4H), 1.86/1.6/1.6/1.34 (m+m/m+m, 4H). HRMS calculated for C₂₈H₂₈NO₃Br: 505.1253; found: 506.1329 (M+H).

The enantiomer eluting later was collected as Example 220. HRMS calculated for C₂₈H₂₈NO₃Br: 505.1253; found: 506.1305 (M+H).

### Example 221

### Example 221A 2-(3-chlorophenyl)-1H-indene

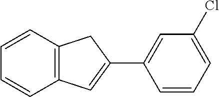 [SMILES: Clc1cccc(C2=Cc3ccccc3C2)c1]

To an oven-dried microwave vial was added Example 212A (500 mg, 2.07 mmol, 1 eq), 3-chloroiodobenzene (0.28 mL, 2.28 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The reaction was partitioned between EtOAc and sat. aq. NaHCO₃ solution, and the organic phase was washed with brine, dried (MgSO₄) and concentrated in vacuo. Purification by flash chromatography (20 g silica cartridge) eluting with a gradient of 0-5% DCM in heptane afforded Example 221A as a white solid (317 mg, 1.40 mmol, 68%). ¹H NMR (400 MHz, DMSO-d₆) δ ppm: 7.77 (t, J=1.9 Hz, 1H), 7.71-7.67 (m, 1H), 7.54-7.48 (m, 2H), 7.47-7.41 (m, 2H), 7.37-7.33 (m, 1H), 7.31-7.26 (m, 1H), 7.21 (td, J=7.4, 1.2 Hz, 1H), 3.90-3.85 (m, 2H).

### Example 221B 2″-(3-chlorophenyl)dispiro[[1,3]dioxolane-2,1′-cyclohexane-4′,1″-indene]

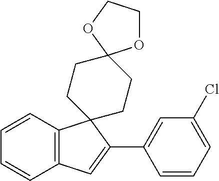 [SMILES: Clc1cccc(C2=Cc3ccccc3C23CCC2(CC3)OCCO2)c1]

Using General procedure 8a and Example 221A (300 mg, 1.32 mmol, 1 eq) as the appropriate indene, purification by flash chromatography (20 g silica cartridge) eluting with a gradient of 0-10% EtOAc in heptane afforded Example 221B as a golden gum (379 mg, 1.07 mmol, 81%). ¹H NMR (400 MHz, DMSO-d₆) δ ppm: 7.78-7.73 (m, 1H), 7.72-7.69 (m, 1H), 7.64-7.59 (m, 1H), 7.51-7.40 (m, 3H), 7.35-7.29 (m, 1H), 7.26-7.19 (m, 2H), 3.97 (s, 4H), 2.34 (td, J=13.6, 4.5 Hz, 2H), 2.16 (td, J=13.5, 4.6 Hz, 2H), 1.86-1.77 (m, 2H), 1.32-1.23 (m, 2H).

### Example 221C 2″-(3-chlorophenyl)-2″,3″-dihydrodispiro[[1,3]dioxolane-2,1′-cyclohexane-4′,1″-indene]

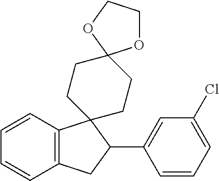 [SMILES: Clc1cccc(C2Cc3ccccc3C23CCC2(CC3)OCCO2)c1]

To a solution of Example 221B (378 mg, 1.07 mmol, 1 eq) in AcOH (15 mL) was added 5% Pt/C (209 mg) under N₂. The mixture was evacuated and backfilled with N₂ (×3), then evacuated and filled with H₂ and shaken at rt for 4 h under an atmosphere of H₂. The mixture was diluted with 5% aq. NH₃ solution and extracted with EtOAc. The combined organic extracts were washed with 5% aq. NH₃ solution, brine, dried (MgSO₄) and concentrated in vacuo to afford a racemate, Example 221C, isolated as a colourless glass (332 mg, 0.94 mmol, 87%). ¹H NMR (400 MHz, DMSO-d₆) δ ppm: 7.34-7.17 (m, 6H), 7.15-7.09 (m, 1H), 7.02-6.95 (m, 1H), 3.91-3.77 (m, 4H), 3.62-3.57 (m, 1H), 3.48-3.39 (m, 1H), 2.97-2.88 (m, 1H), 1.99-1.67 (m, 4H), 1.67-1.56 (m, 1H), 1.50-1.19 (m, 3H).

### Example 221 4-(3-bromoanilino)-2′-(3-chlorophenyl)-2′,3′-dihydrospiro[cyclohexane-1,1′-indene]-4-carboxylic acid

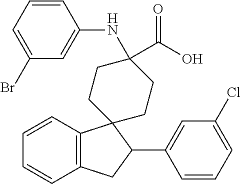 [SMILES: O=C(O)C1(Nc2cccc(Br)c2)CCC2(CC1)c1ccccc1CC2c1cccc(Cl)c1]

To a solution of Example 221C (200 mg, 0.56 mmol, 1 eq) in THE (5 mL) was added 2 M aq. HCl solution (1.69 mL, 3.38 mmol, 6 eq) and the mixture was heated at 60° C. for 16 h in a sealed flask. The mixture was allowed to cool to rt, concentrated in vacuo and partitioned between EtOAc and sat. aq. NaHCO₃ solution. The organic phase was washed with brine, dried (MgSO₄) and concentrated in vacuo to afford an intermediate which was treated according to General Procedure 10 using 3-bromoaniline (47 μL, 0.43 mmol, 1 eq) as the appropriate aniline. The crude product was purified by flash chromatography (10 g silica cartridge) eluting with a gradient of 0-10% EtOAc in DCM followed by preparative HPLC at pH 4 with a gradient of 59-79% MeCN in water to obtain Example 221, as a racemic mixture of diastereoisomers, isolated as a white solid (41.3 mg, 0.08 mmol, 19%). LRMS calculated for C₂₇H₂₅BrClNO₂: 509; found: 510 (M+H). ¹H NMR (400 MHz, DMSO-d₆) δ ppm: 7.34-6.93 (m, 9H), 6.78-6.63 (m, 2H), 6.57-6.53/6.51-6.46 (m, 1H), 3.65-3.31 (m, 2H), 3.04-2.96/2.92-2.85 (m, 1H), 2.41-2.18 (m, 1H), 2.13-1.51 (m, 6H), 1.36-1.06 (m, 1H).

### Example 222

### Example 222A 2-(4-chlorophenyl)-1H-indene

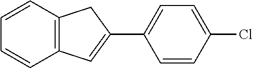 [SMILES: Clc1ccc(C2=Cc3ccccc3C2)cc1]

To an oven-dried microwave vial was added Example 212A (500 mg, 2.07 mmol, 1 eq), 4-chloroiodobenzene (544 mg, 2.28 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The reaction was partitioned between EtOAc and sat. aq. NaHCO₃ solution, and the organic phase was washed with brine, dried (MgSO₄) and concentrated in vacuo. Purification by flash chromatography (20 g silica cartridge) eluting with a gradient of 0-5% DCM in heptane afforded Example 222A as a white solid (349 mg, 1.54 mmol, 75%). ¹H NMR (400 MHz, DMSO-d₆) δ ppm: 7.77-7.72 (m, 2H), 7.52-7.41 (m, 5H), 7.31-7.25 (m, 1H), 7.22-7.17 (m, 1H), 3.87-3.83 (m, 2H).

### Example 222B 2″-(4-chlorophenyl)dispiro[[1,3]dioxolane-2,1′-cyclohexane-4′,1″-indene]

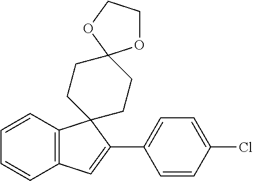 [SMILES: Clc1ccc(C2=Cc3ccccc3C23CCC2(CC3)OCCO2)cc1]

Using General procedure 8a and Example 222A (300 mg, 1.32 mmol, 1 eq) as the appropriate indene, Example 222B was isolated as an off-white solid (370 mg, 1.05 mmol, 79%). ¹H NMR (400 MHz, DMSO-d₆) δ ppm: 7.76-7.71 (m, 1H), 7.65-7.60 (m, 2H), 7.53-7.48 (m, 2H), 7.46-7.42 (m, 1H), 7.34-7.28 (m, 1H), 7.21 (td, J=7.5, 1.3 Hz, 1H), 7.11 (s, 1H), 3.98-3.93 (m, 4H), 2.38-2.26 (m, 2H), 2.16 (td, J=13.5, 4.5 Hz, 2H), 1.85-1.76 (m, 2H), 1.31-1.22 (m, 2H).

### Example 222C 2″-(4-chlorophenyl)-2″,3″-dihydrodispiro[[1,3]dioxolane-2,1′-cyclohexane-4′,1″-indene]

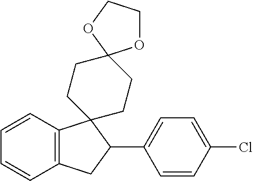 [SMILES: Clc1ccc(C2Cc3ccccc3C23CCC2(CC3)OCCO2)cc1]

To a solution of Example 222B (268 mg, 0.76 mmol, 1 eq) in AcOH (10 mL) was added 5% Pt/C (148 mg) under N₂. The mixture was evacuated and backfilled with N₂ (×3), then evacuated and filled with H₂ and shaken at rt for 4 h under an atmosphere of H₂. The mixture was diluted with 5% aq. NH₃ solution and extracted with EtOAc. The combined organic extracts were washed with 5% aq. NH₃ solution, brine, dried (MgSO₄) and concentrated in vacuo to afford a racemate, Example 222C, isolated as a white solid (236 mg, 0.67 mmol, 88%). ¹H NMR (400 MHz, DMSO-d₆) δ ppm: 7.32-7.17 (m, 6H), 7.10-7.02 (m, 2H), 3.89-3.77 (m, 4H), 3.57 (dd, J=7.7, 3.3 Hz, 1H), 3.43 (dd, J=16.2, 7.7 Hz, 1H), 2.90 (dd, J=16.2, 3.3 Hz, 1H), 1.96-1.70 (m, 4H), 1.66-1.55 (m, 1H), 1.47-1.17 (m, 3H).

### Example 222 4-(3-bromoanilino)-2′-(4-chlorophenyl)-2′,3′-dihydrospiro[cyclohexane-1,1′-indene]-4-carboxylic acid

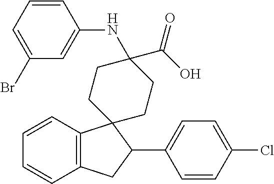 [SMILES: O=C(O)C1(Nc2cccc(Br)c2)CCC2(CC1)c1ccccc1CC2c1ccc(Cl)cc1]

To a solution of Example 222C (200 mg, 0.56 mmol, 1 eq) in THE (5 mL) was added 2 M aq. HCl solution (1.69 mL, 3.38 mmol, 6 eq) and the mixture was heated at 60° C. for 16 h in a sealed flask. The mixture was allowed to cool to rt, concentrated in vacuo and partitioned between EtOAc and sat. aq. NaHCO₃ solution. The organic phase was washed with brine, dried (MgSO₄) and concentrated in vacuo to afford an intermediate which was treated according to General Procedure 10 using 3-bromoaniline (68 μL, 0.63 mmol, 1.1 eq) as the appropriate aniline. Purification by flash chromatography (10 g silica cartridge) eluting with a gradient of 0-10% EtOAc in DCM followed by preparative HPLC at pH 4 eluting with a gradient of 59-79% MeCN in water afforded a racemic mixture of diastereoisomers, Example 222, isolated as a white solid (90.6 mg, 0.18 mmol, 28%). LRMS calculated for C₂₇H₂₅BrClNO₂: 509; found: 510 (M+H). ¹H NMR (400 MHz, DMSO-d₆) δ ppm: 7.33-7.18 (m, 6H), 7.11-6.92 (m, 3H), 6.77-6.63 (m, 2H), 6.57-6.46 (m, 1H), 3.63-3.33 (m, 2H), 3.02-2.93/2.89-2.82 (m, 1H), 2.43-2.15 (m, 1H), 2.13-1.52 (m, 6H), 1.32-1.05 (m, 1H).

### Example 223

### Example 223A 2-(1H-inden-2-yl)-1,3-thiazole

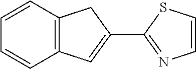 [SMILES: C1=C(c2nccs2)Cc2ccccc21]

To an oven-dried microwave vial was added Example 212A (500 mg, 2.07 mmol, 1 eq), 2-bromothiazole (205 μL, 2.27 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The reaction was partitioned between EtOAc and sat. aq. NaHCO₃ solution, and the organic phase was washed with brine, dried (MgSO₄) and concentrated in vacuo. Purification by flash chromatography (20 g silica cartridge) eluting with a gradient of 0-10% EtOAc in heptane followed by trituration with heptane, afforded Example 223A as a green solid (157 mg, 0.79 mmol, 38%). ¹H NMR (400 MHz, DMSO-d₆) δ ppm: 7.89 (d, J=3.2 Hz, 1H), 7.75 (d, J=3.2 Hz, 1H), 7.60-7.56 (m, 1H), 7.56-7.50 (m, 2H), 7.35-7.25 (m, 2H), 3.97-3.94 (m, 2H).

### Example 223B 2-(dispiro[[1,3]dioxolane-2,1′-cyclohexane-4′,1″-inden]-2″-yl)-1,3-thiazole

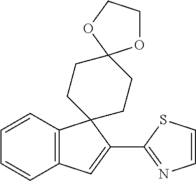 [SMILES: C1=C(c2nccs2)C2(CCC3(CC2)OCCO3)c2ccccc21]

Using General procedure 8a and Example 223A (157 mg, 0.79 mmol, 1 eq) as the appropriate indene afforded Example 223B as an orange solid (152 mg, 0.47 mmol, 59%). ¹H NMR (400 MHz, DMSO-d₆) δ ppm: 7.96 (d, J=3.3 Hz, 1H), 7.79-7.73 (m, 2H), 7.57-7.53 (m, 2H), 7.39-7.33 (m, 1H), 7.32-7.26 (m, 1H), 4.04-3.93 (m, 4H), 3.02 (td, J=13.8, 4.3 Hz, 2H), 2.19 (td, J=14.0, 4.5 Hz, 2H), 1.89-1.81 (m, 2H), 1.22-1.14 (m, 2H).

### Example 223C 2-(2″,3″-dihydrodispiro[[1,3]dioxolane-2,1′-cyclohexane-4′,1″-inden]-2″-yl)-1,3-thiazole

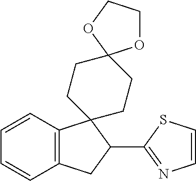 [SMILES: c1ccc2c(c1)CC(c1nccs1)C21CCC2(CC1)OCCO2]

To a solution of Example 223B (152 mg, 0.47 mmol, 1 eq) in EtOH (4 mL) and THE (4 mL) was added catalytic 10% Pd/C under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1) The combined filtrates were concentrated in vacuo to afford a racemate, Example 223C, isolated as a white solid (105 mg, 0.32 mmol, 69%). ¹H NMR (400 MHz, DMSO-d₆) δ ppm: 7.67 (d, J=3.3 Hz, 1H), 7.46 (dd, J=3.4, 0.7 Hz, 1H), 7.36-7.29 (m, 1H), 7.26-7.20 (m, 3H), 4.13-4.07 (m, 1H), 3.91-3.8 (m, 4H), 3.55-3.46 (m, 1H), 3.07 (dd, J=16.2, 3.6 Hz, 1H), 1.97-1.74 (m, 4H), 1.71-1.54 (m, 2H), 1.49-1.40 (m, 1H), 1.21-1.11 (m, 1H).

### Example 223 4-(3-bromoanilino)-2′-(1,3-thiazol-2-yl)-2′,3′-dihydrospiro[cyclohexane-1,1′-indene]-4-carboxylic acid, diastereoisomer 1

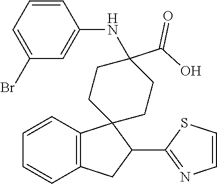 [SMILES: O=C(O)C1(Nc2cccc(Br)c2)CCC2(CC1)c1ccccc1CC2c1nccs1]

Using General procedure 9 and Example 223C (21 mg, 0.06 mmol, 1 eq) as the appropriate ketal, afforded an intermediate which was treated according to General Procedure 10 using 3-bromoaniline (12 μL, 0.11 mmol, 2 eq) as the appropriate aniline. A racemic mixture of diastereoisomers was obtained. Purification using a DCM-wet PE-AX cartridge (5 g), washing with DCM, MeOH and eluting with 5% HCOOH in DCM, followed by preparative HPLC at pH 4 eluting with a gradient of 53-63% MeCN in water, afforded the major isomer as a single racemic diastereoisomer, Example 223, isolated as a beige powder (2.3 mg, 8%). LRMS calculated for C₂₄H₂₃BrN₂O₂S: 482; found: 483 (M+H). ¹H NMR (400 MHz, DMSO-d₆) δ ppm: 12.62 (br s, 1H), 7.65 (d, J=3.3 Hz, 1H), 7.44 (d, J=3.3 Hz, 1H), 7.34-7.30 (m, 1H), 7.28-7.17 (m, 3H), 6.98 (t, J=8.0 Hz, 1H), 6.77 (t, J=2.1 Hz, 1H), 6.69-6.63 (m, 1H), 6.58-6.53 (m, 1H), 6.18 (br s, 1H), 4.15 (dd, J=7.1, 1.9 Hz, 1H), 3.55 (dd, J=16.2, 7.1 Hz, 1H), 3.06-2.97 (m, 1H), 2.27-2.16 (m, 1H), 2.14-1.99 (m, 2H), 1.95-1.84 (m, 1H), 1.82-1.61 (m, 3H), 1.04-0.96 (m, 1H).

### Example 224 (1r,4r)-4-(3-chloroanilino)-2′-(4-methoxy-3-methylphenyl)spiro[cyclohexane-1,1′-indene]-4-carboxylic acid

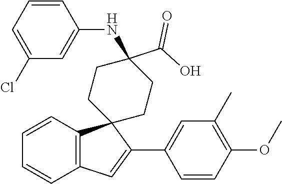 [SMILES: COc1ccc(C2=Cc3ccccc3[C@]23CC[C@](Nc2cccc(Cl)c2)(C(=O)O)CC3)cc1C]

Using General procedure 18 and Preparation 6b as the appropriate 2-bromo-indene and (4-methoxy-3-methyl-phenyl)boronic acid as the appropriate boronic acid, Example 224 was obtained. HRMS calculated for C₂₉H₂₈NO₃Cl: 473.1758; found: 474.1839 (M+H).

### Example 225 (1s,4s)-4-(3-chloroanilino)-2′-(4-methoxy-3-methylphenyl)spiro[cyclohexane-1,1′-indene]-4-carboxylic acid

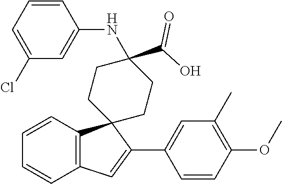 [SMILES: COc1ccc(C2=Cc3ccccc3[C@]23CC[C@@](Nc2cccc(Cl)c2)(C(=O)O)CC3)cc1C]

Using General procedure 18 and Preparation 6a as the appropriate 2-bromo-indene and (4-methoxy-3-methyl-phenyl)boronic acid as the appropriate boronic acid, Example 225 was obtained. ¹H NMR (500 MHz, DMSO-d₆) δ ppm: 12 (br s, 1H), 7.77 (dm, 1H), 7.63 (m, 1H), 7.63 (m, 1H), 7.39 (dm, 1H), 7.27 (m, 1H), 7.17 (m, 1H), 7.13 (s, 1H), 7.10 (t, 1H), 6.92 (d, 1H), 6.74 (t, 1H), 6.63 (dm, 1H), 6.57 (dm, 1H), 6.56 (br s, 1H), 3.82 (s, 3H), 2.55/1.00 (m+m, 4H), 2.45/2.27 (m+m, 4H), 2.21 (s, 3H). HRMS calculated for C₂₉H₂₈NO₃Cl: 473.1758; found: 474.1842 (M+H).

### Example 226 (1r,4r)-4-(3-chloroanilino)-2′-(3-chloro-4-methoxyphenyl)spiro[cyclohexane-1,1′-indene]-4-carboxylic acid

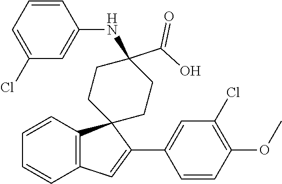 [SMILES: COc1ccc(C2=Cc3ccccc3[C@]23CC[C@](Nc2cccc(Cl)c2)(C(=O)O)CC3)cc1Cl]

Using General procedure 18 and Preparation 6b as the appropriate 2-bromo-indene and (3-chloro-4-methoxy-phenyl)boronic acid as the appropriate boronic acid, Example 226 was obtained. HRMS calculated for C₂₈H₂₅NO₃Cl₂: 493.1212; found: 494.1277 (M+H).

### Example 227 (1s,4s)-4-(3-chloroanilino)-2′-(3-chloro-4-methoxyphenyl)spiro[cyclohexane-1,1′-indene]-4-carboxylic acid

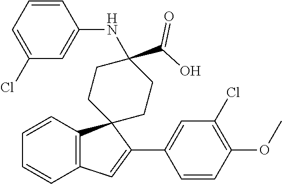 [SMILES: COc1ccc(C2=Cc3ccccc3[C@]23CC[C@@](Nc2cccc(Cl)c2)(C(=O)O)CC3)cc1Cl]

Using General procedure 18 and Preparation 6a as the appropriate 2-bromo-indene and (3-chloro-4-methoxy-phenyl)boronic acid as the appropriate boronic acid, Example 227 was obtained. HRMS calculated for C₂₈H₂₅NO₃Cl₂: 493.1212; found: 494.1291 (M+H).

### Example 228 (1r,4r)-4-(3-chloroanilino)-2′-(4-methoxy-2-methylphenyl)spiro[cyclohexane-1,1′-indene]-4-carboxylic acid

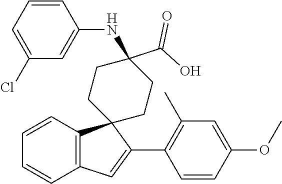 [SMILES: COc1ccc(C2=Cc3ccccc3[C@]23CC[C@](Nc2cccc(Cl)c2)(C(=O)O)CC3)c(C)c1]

Using General procedure 18 and Preparation 6b as the appropriate 2-bromo-indene and (4-methoxy-2-methyl-phenyl)boronic acid as the appropriate boronic acid, Example 228 was obtained. HRMS calculated for C₂₉H₂₈NO₃Cl: 473.1758; found: 474.1852 (M+H).

### Example 229 (1s,4s)-4-(3-chloroanilino)-2′-(4-methoxy-2-methylphenyl)spiro[cyclohexane-1,1′-indene]-4-carboxylic acid

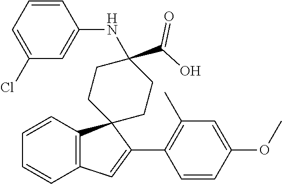 [SMILES: COc1ccc(C2=Cc3ccccc3[C@]23CC[C@@](Nc2cccc(Cl)c2)(C(=O)O)CC3)c(C)c1]

Using General procedure 18 and Preparation 6a as the appropriate 2-bromo-indene and (4-methoxy-2-methyl-phenyl)boronic acid as the appropriate boronic acid, Example 229 was obtained. ¹H NMR (500 MHz, DMSO-d₆) δ ppm: 11.94 (br s, 1H), 7.75 (d, 1H), 7.36 (d, 1H), 7.28 (t, 1H), 7.20 (t, 1H), 6.99 (d, 1H), 6.98 (t, 1H), 6.87 (d, 1H), 6.76 (dd, 1H), 6.50 (s, 1H), 6.49 (dd, 1H), 6.41 (d, 1H), 6.35 (dd, 1H), 5.99 (br s, 1H), 3.77 (s, 3H), 2.43/2.10 (t/d, 4H), 2.15 (s, 3H), 2.05/1.20 (t/d, 4H). HRMS calculated for C₂₉H₂₈NO₃Cl: 473.1758; found: 474.1833 (M+H).

### Example 230 (1s,4s)-4-(3-chloroanilino)-2′-(4-methoxyphenyl)spiro[cyclohexane-1,1′-indene]-4-carboxylic acid

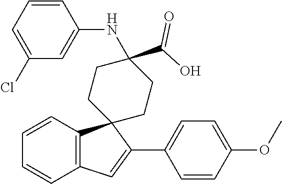 [SMILES: COc1ccc(C2=Cc3ccccc3[C@]23CC[C@@](Nc2cccc(Cl)c2)(C(=O)O)CC3)cc1]

Using General procedure 18 and Preparation 6a as the appropriate 2-bromo-indene and (4-methoxy-phenyl)boronic acid as the appropriate boronic acid, Example 229 was obtained. HRMS calculated for C₂₈H₂₆NO₃Cl: 459.1601; found: 460.1675 (M+H).

### Example 231

### Example 231A 4-(dispiro[[1,3]dioxolane-2,1′-cyclohexane-4′,1″-inden]-2″-yl)-3,5-dimethyl-1,2-oxazole

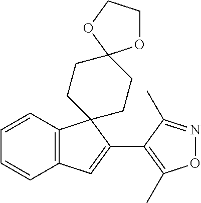 [SMILES: Cc1noc(C)c1C1=Cc2ccccc2C12CCC1(CC2)OCCO1]

To an oven-dried microwave vial was added Preparation 4aA (150 mg, 0.47 mmol, 1 eq), 3,5-dimethyl-4-(tetramethyl-1,3,2-dioxaborolan-2-yl)-1,2-oxazole (125 mg, 0.56 mmol, 1.2 eq) and Cs₂CO₃ (304 mg, 0.93 mmol, 2 eq) in 1,4-dioxane (3 mL) and water (0.75 mL). The mixture was sparged with N₂, followed by the addition of Pd(dppf)Cl₂×DCM (38 mg, 0.05 mmol, 0.1 eq) and then heated at 120° C. for 2 h under microwave irradiation. The mixture was partitioned between EtOAc and sat. aq. NaHCO₃ solution, and the organic phase was washed with brine, dried (MgSO₄) and concentrated in vacuo. Purification by flash chromatography (20 g silica cartridge) eluting with a gradient of 0-20% EtOAc in heptane afforded Example 231A as an off-white powder (99 mg, 0.29 mmol, 63%). LRMS calculated for C₂₁H₂₃NO₃: 337; found: 338 (M+H).

### Example 231 4-(3-bromoanilino)-2′-(3,5-dimethyl-1,2-oxazol-4-yl)-2′,3′-dihydrospiro[cyclohexane-1,1′-indene]-4-carboxylic acid, diastereoisomer 1

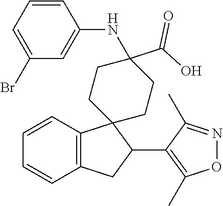 [SMILES: Cc1noc(C)c1C1Cc2ccccc2C12CCC(Nc1cccc(Br)c1)(C(=O)O)CC2]

To a solution of Example 231A (99 mg, 0.29 mmol, 1 eq) in EtOH (3 mL) and THE (3 mL) was added catalytic 10% Pd/C under N₂. The mixture was evacuated and backfilled with N₂ (×3), then evacuated and filled with H₂ and shaken at rt for 16 h under an atmosphere of H₂.

The mixture was filtered through a celite pad, washed with EtOH:THF (1:1). The combined filtrates were concentrated in vacuo. Purification by flash chromatography (5 g silica cartridge) eluting with a gradient of 0-20% EtOAc in heptane afforded an intermediate which was treated according to General procedure 9 and then General procedure 10 using 3-bromoaniline (22 μL, 0.2 mmol, 1 eq) as the appropriate aniline. A racemic mixture of diastereoisomers was obtained. Purification using a DCM-wet PE-AX cartridge (5 g), washing with DCM, MeOH and eluting in 5% HCOOH in DCM, followed by preparative HPLC at pH 4, eluting with a gradient of 53-73% MeCN in water, afforded the major isomer as a racemic single diastereoisomer, Example 231, isolated as a cream powder (4.9 mg, 0.01 mmol, 5%). LRMS calculated for C₂₆H₂₇BrN₂O₃: 494; found: 495 (M+H). ¹H NMR (400 MHz, DMSO-d₆) δ ppm: 7.31-7.19 (m, 4H), 6.99 (t, J=8.1 Hz, 1H), 6.74 (t, J=2.0 Hz, 1H), 6.70-6.66 (m, 1H), 6.57-6.52 (m, 1H), 3.41-3.30 (m, 2H), 2.95-2.85 (m, 1H), 2.48-2.37 (m, 1H), 2.21-2.06 (m, 4H), 1.93-1.67 (m, 6H), 1.66-1.55 (m, 1H), 1.50-1.32 (m, 2H).

### Example 232

### Example 232A 2″-(5-methylthiophen-3-yl)dispiro[[1,3]dioxolane-2,1′-cyclohexane-4′,1″-indene]

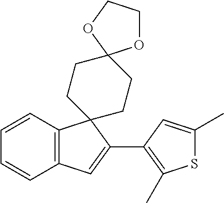 [SMILES: Cc1cc(C2=Cc3ccccc3C23CCC2(CC3)OCCO2)c(C)s1]

To an oven-dried microwave vial was added Example 212A (500 mg, 2.07 mmol, 1 eq), 4-bromo-2-methylthiophene (205 μL, 2.27 mmol, 1.1 eq) and Cs₂CO₃ (1.35 g, 4.13 mmol, 2 eq) in 1,4-dioxane (10 mL) and water (2.5 mL). The mixture was sparged with N₂, followed by the addition of Pd(dppf)Cl₂×DCM (169 mg, 0.21 mmol, 0.1 eq) and then heated at 120° C. for 1 h under microwave irradiation. The mixture was partitioned between EtOAc and sat. aq. NaHCO₃ solution, and the organic phase was washed with brine, dried (MgSO₄) and concentrated in vacuo. Purification by flash chromatography (20 g silica cartridge) eluting with a gradient of 0-10% EtOAc in heptane and then trituration in heptane, afforded an intermediate which was treated according to General procedure 8a to afford Example 232A, isolated as an orange solid (128 mg, 0.38 mmol, 38%). LRMS calculated for C₂₁H₂₂O₂S: 338; found: 339 (M+H). ¹H NMR (400 MHz, DMSO-d₆) δ ppm: 7.75 (d, J=7.5 Hz, 1H), 7.45-7.39 (m, 2H), 7.33-7.26 (m, 2H), 7.21-7.14 (m, 2H), 4.07-3.97 (m, 4H), 2.48 (d, J=1.1 Hz, 3H), 2.18 (td, J=13.8, 4.7 Hz, 2H), 1.88-1.80 (m, 2H), 1.19-1.11 (m, 2H).

### Example 232B 2″-(5-methylthiophen-3-yl)-2″,3″-dihydrodispiro[[1,3]dioxolane-2,1′-cyclohexane-4′,1″-indene]

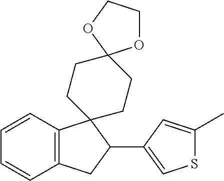 [SMILES: Cc1cc(C2Cc3ccccc3C23CCC2(CC3)OCCO2)cs1]

To a solution of Example 232A (128 mg, 0.38 mmol, 1 eq) in EtOH (4 mL) and THE (4 mL) was added catalytic 10% Pd/C under N₂. The mixture was evacuated and backfilled with N₂ (×3), then evacuated and filled with H₂ and shaken at rt for 16 h under an atmosphere of H₂. The mixture was filtered through a celite pad, washed with EtOH:THF (1:1). The combined filtrates were concentrated in vacuo. Purification by flash chromatography (5 g silica cartridge) eluting with a gradient of 0-20% EtOAc in heptane afforded a racemate, Example 232B, isolated as a colourless oil (126 mg, 0.37 mmol, 98%). ¹H NMR (400 MHz, DMSO-d₆) δ ppm: 7.30-7.15 (m, 4H), 6.92 (d, J=1.4 Hz, 1H), 6.22-6.17 (m, 1H), 3.92-3.78 (m, 4H), 3.58 (dd, J=7.4, 3.8 Hz, 1H), 3.36-3.27 (m, 1H), 2.83 (dd, J=16.0, 3.8 Hz, 1H), 2.30 (d, J=1.1 Hz, 3H), 1.93-1.71 (m, 4H), 1.64-1.42 (m, 3H), 1.36-1.22 (m, 1H).

### Example 232 4-(3-bromoanilino)-2′-(5-methylthiophen-3-yl)-2′,3′-dihydrospiro[cyclohexane-1,1′-indene]-4-carboxylic acid

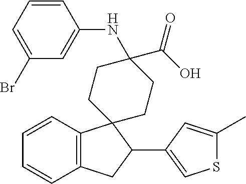 [SMILES: Cc1cc(C2Cc3ccccc3C23CCC(Nc2cccc(Br)c2)(C(=O)O)CC3)cs1]

Using General procedure 9 and Example 232B (126 mg, 0.37 mmol, 1 eq) as the appropriate ketal afforded an intermediate which was treated according to General Procedure 10 using 3-bromoaniline (38 μL, 0.35 mmol, 1 eq) as the appropriate aniline. A racemic mixture of diastereoisomers was obtained. Purification using a DCM-wet PE-AX cartridge (5 g), washing with DCM, MeOH and eluting with 5% HCOOH in DCM, and then preparative HPLC at pH 4, eluting with a gradient of 65-85% MeCN in water, afforded a racemic mixture of diastereoisomers, Example 232, isolated as a colourless solid (30.3 mg, 0.06 mmol, 18%). LRMS calculated for C₂₆H₂₆BrNO₂S: 495; found: 496 (M+H). ¹H NMR (400 MHz, DMSO-d₆) δ ppm: δ 7.36-7.15 (m, 4H), 7.02-6.94 (m, 1H), 6.92-6.89/6.87-6.84 (m, 1H), 6.79-6.64 (m, 2H), 6.59-6.54/6.51-6.46 (m, 1H), 6.42-6.37/6.16-6.13 (m, 1H), 3.65-3.19 (m, 2H), 3.00-2.90/2.84-2.75 (m, 1H), 2.43-1.11 (m, 11H).

### Example 233

### Example 233A 2-(3,4-dichlorophenyl)-1H-indene

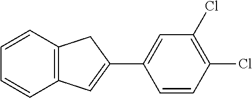 [SMILES: Clc1ccc(C2=Cc3ccccc3C2)cc1Cl]

To a solution of 1H-inden-2-ylboronic acid (500 mg, 3.13 mmol, 1 eq) in 1,4-dioxane (10 mL) and water (2.5 mL) was added Cs₂CO₃ (2.01 g, 6.16 mmol, 2 eq) and 4-bromo-1,2-dichlorobenzene (778 mg, 3.44 mmol, 1.1 eq). The mixture was sparged with N₂ followed by the addition of Pd(dppf)Cl₂×DCM (256 mg, 0.31 mmol) and then heated at 120° C. for 1 h under microwave irradiation. The mixture was concentrated in vacuo, partitioned between Et₂O and sat. aq. NaHCO₃ solution and the organic phase was washed with brine, dried (MgSO₄) and concentrated in vacuo. Purification by flash chromatography (20 g silica cartridge) eluting with heptane afforded Example 233A as an off-white solid (469 mg, 1.80 mmol, 58%). ¹H NMR (400 MHz, DMSO-d₆) δ ppm: 7.96 (d, J=2.0 Hz, 1H), 7.72 (dd, J=8.4, 2.0 Hz 1H), 7.66 (d, J=8.4 Hz, 1H), 7.58-7.55 (m, 1H), 7.53-7.48 (m, 1H), 7.47-7.43 (m, 1H), 7.32-7.26 (m, 1H), 7.25-7.19 (m, 1H), 3.90-3.85 (m, 2H).

### Example 233B 2″-(3,4-dichlorophenyl)dispiro[[1,3]dioxolane-2,1′-cyclohexane-4′,1″-indene]

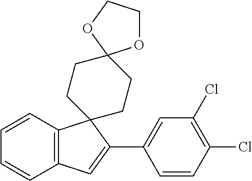 [SMILES: Clc1ccc(C2=Cc3ccccc3C23CCC2(CC3)OCCO2)cc1Cl]

Using General procedure 8a and Example 233A (356 mg, 1.36 mmol, 1 eq) as the appropriate indene, purification by flash chromatography (20 g silica cartridge) eluting with a gradient of 0-50% DCM in heptane afforded Example 233B as an off-white solid (398 mg, 1.03 mmol, 76%). ¹H NMR (400 MHz, DMSO-d₆) δ ppm: 7.93 (d, J=2.0 Hz, 1H), 7.79-7.74 (m, 1H), 7.71 (d, J=8.6 Hz, 1H), 7.67 (dd, J=8.6, 2.0 Hz, 1H), 7.48-7.44 (m, 1H), 7.35-7.28 (m, 2H), 7.24 (td, J=7.5, 1.3 Hz, 1H), 3.98 (s, 4H), 2.33 (td, J=13.6, 4.5 Hz, 2H), 2.16 (td, J=13.6, 4.6 Hz, 2H), 1.87-1.77 (m, 2H), 1.32-1.22 (m, 2H).

### Example 233C 2′-(3,4-dichlorophenyl)spiro[cyclohexane-1,1′-inden]-4-one

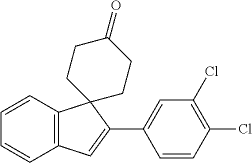 [SMILES: O=C1CCC2(CC1)C(c1ccc(Cl)c(Cl)c1)=Cc1ccccc12]

To a solution of Example 233B (100 mg, 0.26 mmol, 1 eq) in THE (2.5 mL) was added 2 M aq. HCl solution (0.77 mL, 1.55 mmol, 6 eq) and the mixture was heated at 100° C. for 1 h under microwave irradiation. The mixture was allowed to cool to rt and partitioned between DCM and sat. aq. NaHCO₃ solution. The organic phase was washed with brine, dried (MgSO₄) and concentrated in vacuo to afford Example 233C as an off-white solid (86.2 mg, 0.25 mmol, 97%) that was used directly in the subsequent step without further purification. ¹H NMR (400 MHz, DMSO-d₆) δ ppm: 7.93-7.88 (m, 1H), 7.77 (d, J=2.1 Hz, 1H), 7.68 (d, J=8.4 Hz, 1H), 7.54-7.46 (m, 2H), 7.38-7.33 (m, 1H), 7.25 (td, J=7.5, 1.3 Hz, 1H), 7.13 (s, 1H), 3.00-2.88 (m, 2H), 2.48-2.35 (m, 4H), 1.69-1.58 (m, 2H).

### Example 233 4-(3-bromoanilino)-2′-(3,4-dichlorophenyl)spiro[cyclohexane-1,1′-indene]-4-carboxylic acid, diastereoisomer 1

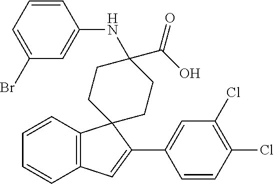 [SMILES: O=C(O)C1(Nc2cccc(Br)c2)CCC2(CC1)C(c1ccc(Cl)c(Cl)c1)=Cc1ccccc12]

Using General Procedure 10 and Example 233C (86.2 mg, 0.25 mmol, 1 eq) as the appropriate ketone and 3-bromoaniline (0.03 mL, 0.26 mmol, 1 eq) as the appropriate aniline, a mixture of diastereoisomers was obtained. Purification by preparative HPLC at pH 4 eluting with a gradient of 80-90% MeCN in water, afforded the major isomer as a single diastereoisomer, Example 233, isolated as a yellow solid (3.76 mg, 0.01 mmol, 3%). LRMS calculated for C₂₇H₂₂BrCl₂NO₂: 541; found: 542 (M+H). ¹H NMR (400 MHz, DMSO-d₆) δ ppm: 7.86-7.78 (m, 2H), 7.67-7.56 (m, 2H), 7.45 (dd, J=7.4, 1.3 Hz, 1H), 7.32 (td, J=7.2, 0.7 Hz, 1H), 7.25 (td, J=7.4, 1.3 Hz, 1H), 7.16 (s, 1H), 7.01 (t, J=8.1 Hz, 1H), 6.84 (t, J=2.0 Hz, 1H), 6.72-6.63 (m, 2H), 2.48-2.38 (m, 2H), 2.36-2.22 (m, 2H), 2.21-2.07 (m, 2H), 1.44-1.32 (m, 2H).

### Example 234

### Example 234A 2-(3-chloro-4-methylphenyl)-1H-indene

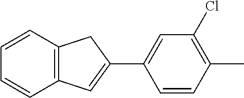 [SMILES: Cc1ccc(C2=Cc3ccccc3C2)cc1Cl]

To a solution of 1H-inden-2-ylboronic acid (500 mg, 3.13 mmol, 1 eq) in 1,4-dioxane (10 mL) and water (2.5 mL) was added Cs₂CO₃ (2.01 g, 6.16 mmol, 2 eq) and 4-bromo-2-chloro-1-methylbenzene (707 mg, 3.44 mmol, 1.1 eq). The mixture was sparged with N₂ followed by the addition of Pd(dppf)Cl₂×DCM (256 mg, 0.31 mmol) and then heated at 120° C. under microwave irradiation. The mixture was concentrated in vacuo and partitioned between Et₂O and sat. aq. NaHCO₃ solution. The organic phase was washed with brine, dried (MgSO₄) and concentrated in vacuo. Purification by flash chromatography (20 g silica cartridge) eluting with heptane afforded Example 234A, isolated as an off-white solid (464 mg, 1.93 mmol, 62%). ¹H NMR (400 MHz, DMSO-d₆) δ ppm: 7.74 (d, J=1.9 Hz, 1H), 7.60 (dd, J=7.9, 1.9 Hz, 1H), 7.51-7.36 (m, 4H), 7.30-7.24 (m, 1H), 7.19 (td, J=7.4, 1.2 Hz, 1H), 3.86-3.83 (m, 2H), 2.35 (s, 3H).

### Example 234B 2″-(3-chloro-4-methylphenyl)dispiro[[1,3]dioxolane-2,1′-cyclohexane-4′,1″-indene]

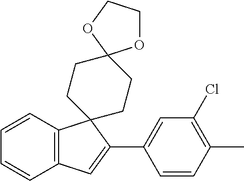 [SMILES: Cc1ccc(C2=Cc3ccccc3C23CCC2(CC3)OCCO2)cc1Cl]

Using General procedure 8a and Example 234A (328 mg, 1.36 mmol, 1 eq) as the appropriate indene, purification by flash chromatography (20 g silica cartridge) eluting with a gradient of 0-50% DCM in heptane afforded Example 234B as an off-white solid (360 mg, 0.98 mmol, 70%). ¹H NMR (400 MHz, DMSO-d₆) δ ppm: 7.76-7.71 (m, 1H), 7.69 (d. J=1.9 Hz, 1H), 7.53 (dd, J=7.9, 1.9 Hz, 1H), 7.45-7.39 (m, 2H), 7.33-7.28 (m, 1H), 7.21 (td, J=7.5, 1.3 Hz, 1H), 7.16 (s, 1H), 3.97 (s, 4H), 2.41-2.29 (m, 5H), 2.15 (td, J=13.6, 4.6 Hz, 2H), 1.86-1.77 (m, 2H), 1.30-1.21 (m, 2H).

### Example 234C 2′-(3-chloro-4-methylphenyl)spiro[cyclohexane-1,1′-inden]-4-one

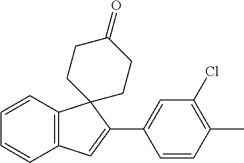 [SMILES: Cc1ccc(C2=Cc3ccccc3C23CCC(=O)CC3)cc1Cl]

To a solution of Example 234B (94.7 mg, 0.26 mmol, 1 eq) in THF (2.5 mL) was added 2 M aq. HCl solution (0.77 mL, 1.55 mmol, 6 eq) and the mixture was heated at 100° C. for 1 h under microwave irradiation. The mixture was allowed to cool to rt and partitioned between DCM and sat. aq. NaHCO₃ solution. The organic phase was washed with brine, dried (MgSO₄) and concentrated in vacuo to afford Example 234C, isolated as an off-white wax (80.8 mg, 0.25 mmol, 97%) that was used directly in the subsequent step without further purification. ¹H NMR (400 MHz, DMSO-d₆) δ ppm: 7.92-7.87 (m, 1H), 7.51 (d, J=1.7 Hz, 1H), 7.48-7.31 (m, 4H), 7.23 (td, J=7.5, 1.3 Hz, 1H), 7.03 (s, 1H), 3.00-2.87 (m, 2H), 2.47-2.34 (m, 7H), 1.71-1.58 (m, 2H).

### Example 234 4-(3-bromoanilino)-2′-(3-chloro-4-methylphenyl)spiro[cyclohexane-1,1′-indene]-4-carboxylic acid, diastereoisomer 1

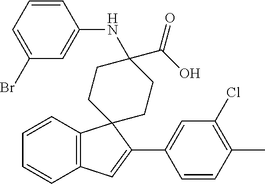 [SMILES: Cc1ccc(C2=Cc3ccccc3C23CCC(Nc2cccc(Br)c2)(C(=O)O)CC3)cc1Cl]

Using General Procedure 10 and Example 234C (80.8 mg, 0.25 mmol, 1 eq) as the appropriate ketone and 3-bromoaniline (0.03 mL, 0.26 mmol, 1 eq) as the appropriate aniline, a mixture of diastereoisomers was obtained. Purification by preparative HPLC at pH 4 eluting with a gradient of 78-85% MeCN in water, afforded the major isomer as a single diastereoisomer, Example 234, isolated as a yellow solid (2.34 mg, 0.01 mmol, 2%). LRMS calculated for C₂₈H₂₅BrClNO₂: 521; found: 522 (M+H). ¹H NMR (400 MHz, DMSO-d₆) δ ppm: 7.79 (d, J=7.6 Hz, 1H), 7.59 (d, J=1.8 Hz, 1H), 7.47-7.40 (m, 2H), 7.36 (d, J=8.1 Hz, 1H), 7.31 (td, J=7.4, 0.8 Hz, 1H), 7.22 (td, J=7.5, 1.3 Hz, 1H), 7.05 (s, 1H), 7.01 (t, J=8.1 Hz, 1H), 6.85 (t, J=2.0 Hz, 1H), 6.73-6.64 (m, 2H), 2.49-2.42 (m, 2H), 2.37 (s, 3H), 2.36-2.25 (m, 2H), 2.14 (td, J=12.8, 3.8 Hz, 2H), 1.41-1.31 (m, 2H).

### Example 235

### Example 235A 2-(4-chloro-3-methylphenyl)-1H-indene

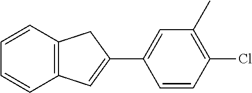 [SMILES: Cc1cc(C2=Cc3ccccc3C2)ccc1Cl]

To a solution of 1H-inden-2-ylboronic acid (500 mg, 3.13 mmol, 1 eq) in 1,4-dioxane (10 mL) and water (2.5 mL) was added Cs₂CO₃ (2.01 g, 6.16 mmol, 2 eq) and 5-bromo-2-chloro-1-methylbenzene (707 mg, 3.44 mmol, 1.1 eq). The mixture was sparged with N₂ followed by the addition of Pd(dppf)Cl₂×DCM (256 mg, 0.31 mmol) and then heated at 120° C. under microwave irradiation. The mixture was concentrated in vacuo and partitioned between Et₂O and sat. aq. NaHCO₃ solution. The organic phase was washed with brine, dried (MgSO₄) and concentrated in vacuo. Purification by flash chromatography (20 g silica cartridge) eluting with heptane afforded Example 235A as an off-white solid (450 mg, 1.87 mmol, 60%). ¹H NMR (400 MHz, DMSO-d₆) δ ppm: 7.74-7.71 (m, 1H), 7.59-7.54 (m, 1H), 7.52-7.47 (m, 1H), 7.46-7.39 (m, 3H), 7.30-7.24 (m, 1H), 7.22-7.16 (m, 1H), 3.85-3.82 (m, 2H), 2.38 (s, 3H).

### Example 235B 2″-(4-chloro-3-methylphenyl)dispiro[[1,3]dioxolane-2,1′-cyclohexane-4′,1″-indene]

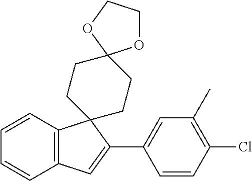 [SMILES: Cc1cc(C2=Cc3ccccc3C23CCC2(CC3)OCCO2)ccc1Cl]

Using General procedure 8a and Example 235A (328 mg, 1.36 mmol, 1 eq) as the appropriate indene, purification by flash chromatography (20 g silica cartridge) eluting with a gradient of 0-50% DCM in heptane afforded Example 235B, isolated as orange crystals (385 mg, 1.05 mmol, 77%). ¹H NMR (400 MHz, DMSO-d₆) δ ppm: 7.77-7.72 (m, 1H), 7.65-7.62 (m, 1H), 7.49-7.46 (m, 2H), 7.45-7.41 (m, 1H), 7.33-7.28 (m, 1H), 7.21 (td, J=7.5, 1.3 Hz, 1H), 7.12 (s, 1H), 3.97 (s, 4H), 2.41-2.29 (m, 5H), 2.16 (td, J=13.5, 4.5 Hz, 2H), 1.86-1.76 (m, 2H), 1.31-1.21 (m, 2H).

### Example 235C 2′-(4-chloro-3-methylphenyl)spiro[cyclohexane-1,1′-inden]-4-one

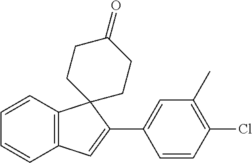 [SMILES: Cc1cc(C2=Cc3ccccc3C23CCC(=O)CC3)ccc1Cl]

To a solution of Example 235B (94.7 mg, 0.26 mmol, 1 eq) in THE (2.5 mL) was added 2 M aq. HCl solution (0.77 mL, 1.55 mmol, 6 eq) and the mixture was heated at 100° C. for 1 h under microwave irradiation. The mixture was allowed to cool to rt and partitioned between DCM and sat. aq. NaHCO₃ solution. The organic phase was washed with brine, dried (MgSO₄) and concentrated in vacuo to afford Example 235C, isolated as an off-white solid (82.2 mg, 0.25 mmol, 99%) that was used directly in the subsequent step without further purification. ¹H NMR (400 MHz, DMSO-d₆) δ ppm: 7.92-7.86 (m, 1H), 7.52-7.42 (m, 3H), 7.38-7.30 (m, 2H), 7.23 (td, J=7.6, 1.3 Hz, 1H), 7.01 (s, 1H), 3.01-2.88 (m, 2H), 2.48-2.35 (m, 7H), 1.69-1.58 (m, 2H).

### Example 235 4-(3-bromoanilino)-2′-(4-chloro-3-methylphenyl)spiro[cyclohexane-1,1′-indene]-4-carboxylic acid, diastereoisomer 1

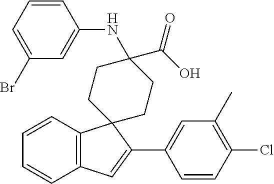 [SMILES: Cc1cc(C2=Cc3ccccc3C23CCC(Nc2cccc(Br)c2)(C(=O)O)CC3)ccc1Cl]

Using General Procedure 10 and Example 235C (82.2 mg, 0.25 mmol, 1 eq) as the appropriate ketone and 3-bromoaniline (0.03 mL, 0.26 mmol, 1 eq) as the appropriate aniline, a mixture of diastereoisomers was obtained. Purification by preparative HPLC at pH 4 eluting with a gradient of 75-85% MeCN in water afforded the major isomer as a single diastereoisomer, Example 235, isolated as a yellow solid (0.81 mg, 0.01 mmol, 1%). LRMS calculated for C₂₈H₂₅BrClNO₂: 521; found: 522 (M+H). ¹H NMR (400 MHz, DMSO-d₆) δ ppm: 7.80 (d, J=7.5 Hz, 1H), 7.66-2.57 (m, 1H), 7.52-7.37 (m, 3H), 7.31 (td, J=7.5, 0.9 Hz, 1H), 7.22 (td, J=7.5, 1.3 Hz, 1H), 7.09 (s, 1H), 7.01 (t, J=8.1 Hz, 1H), 6.86 (t, J=2.1 Hz, 1H), 6.72-6.65 (m, 2H), 2.51-2.32 (m, 7H), 2.19-2.07 (m, 2H), 1.38-1.27 (m, 2H).

### Example 236

### Example 236A 2-(3,5-dichlorophenyl)-1H-indene

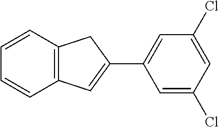 [SMILES: Clc1cc(Cl)cc(C2=Cc3ccccc3C2)c1]

To a solution of 1H-inden-2-ylboronic acid (500 mg, 3.13 mmol, 1 eq) in 1,4-dioxane (10 mL) and water (2.5 mL) was added Cs₂CO₃ (2.01 g, 6.16 mmol, 2 eq) and 1-bromo-3,5-dichlorobenzene (778 mg, 3.44 mmol, 1.1 eq). The mixture was sparged with N₂, followed by the addition of Pd(dppf)Cl₂×DCM (256 mg, 0.31 mmol, 0.1 eq) and then heated at 120° C. under microwave irradiation. The mixture was concentrated in vacuo and partitioned between Et₂O and sat. aq. NaHCO₃ solution. The organic phase was washed with brine, dried (MgSO₄) and concentrated in vacuo. Purification by flash chromatography (20 g silica cartridge) eluting with heptane afforded Example 236A, isolated as an off-white solid (461 mg, 1.77 mmol, 57%). ¹H NMR (400 MHz, DMSO-d₆) δ ppm:7.77 (d, J=1.9 Hz, 2H), 7.65-7.62 (m, 1H), 7.53-7.49 (m, 2H), 7.48-7.44 (m, 1H), 7.33-7.27 (m, 1H), 7.26-7.21 (m, 1H), 3.91-3.87 (m, 2H).

### Example 236B 2″-(3,5-dichlorophenyl)dispiro[[1,3]dioxolane-2,1′-cyclohexane-4′,1″-indene]

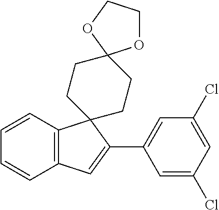 [SMILES: Clc1cc(Cl)cc(C2=Cc3ccccc3C23CCC2(CC3)OCCO2)c1]

Using General procedure 8a and Example 236A (356 mg, 1.36 mmol, 1 eq) as the appropriate indene, purification by flash chromatography (20 g silica cartridge) eluting with a gradient of 0-50% DCM in heptane afforded Example 236B, isolated as orange crystals (433 mg, 1.12 mmol, 82%). ¹H NMR (400 MHz, DMSO-d₆) δ ppm: 7.80-7.74 (m, 3H), 7.60 (t, J=1.8 Hz, 1H), 7.49-7.44 (m, 1H), 7.39 (s, 1H), 7.36-7.30 (m, 1H), 7.25 (td, J=7.5, 1.3 Hz, 1H), 3.99 (s, 4H), 2.33 (td, J=13.6, 4.5 Hz, 2H), 2.16 (td, J=13.5, 4.6 Hz, 2H), 1.88-1.78 (m, 2H), 1.32-1.22 (m, 2H).

### Example 236C 2′-(3,5-dichlorophenyl)spiro[cyclohexane-1,1′-inden]-4-one

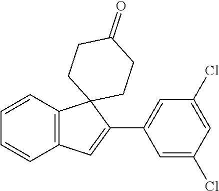 [SMILES: O=C1CCC2(CC1)C(c1cc(Cl)cc(Cl)c1)=Cc1ccccc12]

To a solution of Example 236B (100 mg, 0.26 mmol, 1 eq) in THE (2.5 mL) was added 2 M aq. HCl solution (0.77 mL, 1.55 mmol, 6 eq) and the mixture was heated at 100° C. for 1 h under microwave irradiation. The mixture was allowed to cool to rt and partitioned between DCM and sat. aq. NaHCO₃ solution. The organic phase was washed with brine, dried (MgSO₄) and concentrated in vacuo to afford Example 236C, isolated as an off-white solid (87.7 mg, 0.26 mmol, 99%) that was used directly in the subsequent step without further purification. ¹H NMR (400 MHz, DMSO-d₆) δ ppm: 7.92-7.88 (m, 1H), 7.63 (t, J=1.8 Hz, 1H), 7.53 (d, J=1.8 Hz, 2H), 7.50-7.46 (m, 1H), 7.39-7.33 (m, 1H), 7.26 (td, J=7.5, 1.3 Hz, 1H), 7.12 (s, 1H), 2.99-2.86 (m, 2H), 2.45-2.32 (m, 4H), 1.72-1.60 (m, 2H).

### Example 236 4-(3-bromoanilino)-2′-(3,5-dichlorophenyl)spiro[cyclohexane-1,1′-indene]-4-carboxylic acid, diastereoisomer 1

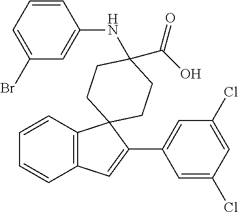 [SMILES: O=C(O)C1(Nc2cccc(Br)c2)CCC2(CC1)C(c1cc(Cl)cc(Cl)c1)=Cc1ccccc12]

Using General Procedure 10 and Example 236C (87.7 mg, 0.26 mmol, 1 eq) as the appropriate ketone and 3-bromoaniline (0.03 mL, 0.26 mmol, 1 eq) as the appropriate aniline, a mixture of diastereoisomers was obtained. Purification by preparative HPLC at pH 4 eluting with a gradient of 75-85% MeCN in water afforded the major isomer as a single diastereoisomer, Example 236, isolated as a yellow solid (4.60 mg, 0.01 mmol, 3%). LRMS calculated for C₂₇H₂₂BrCl₂NO₂: 541; found: 542 (M+H). ¹H NMR (400 MHz, DMSO-d₆) δ ppm: 7.80 (d, J=7.6 Hz, 1H), 7.62 (d, J=1.8 Hz, 2H), 7.59 (t, J=1.8 Hz, 1H), 7.47-7.43 (m, 1H), 7.33 (td, J=7.4, 1.0 Hz, 1H), 7.26 (td, J=7.5, 1.4 Hz, 1H), 7.19 (s, 1H), 7.01 (t, J=8.1 Hz, 1H), 6.83 (t, J=2.1 Hz, 1H), 6.73-6.68 (m, 1H), 6.68-6.63 (m, 1H), 2.48-2.37 (m, 2H), 2.32-2.20 (m, 2H), 2.14 (td, J=12.9, 3.9 Hz, 2H), 1.45-1.35 (m, 2H).

### Example 237

### Example 237A 2-(3,5-dimethoxyphenyl)-1H-indene

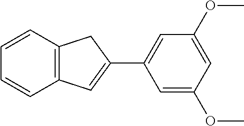 [SMILES: COc1cc(OC)cc(C2=Cc3ccccc3C2)c1]

To a solution of 1H-inden-2-ylboronic acid (500 mg, 3.13 mmol, 1 eq) in 1,4-dioxane (10 mL) and water (2.5 mL) was added Cs₂CO₃ (2.01 g, 6.16 mmol, 2 eq) and 1-bromo-3,5-dimethoxybenzene (747 mg, 3.44 mmol, 1.1 eq). The mixture was sparged with N₂, followed by the addition of Pd(dppf)Cl₂×DCM (256 mg, 0.31 mmol, 0.1 eq) and then heated at 120° C. under microwave irradiation. The mixture was concentrated in vacuo and partitioned between Et₂O and sat. aq. NaHCO₃ solution. The organic phase was washed with brine, dried (MgSO₄) and concentrated in vacuo. Purification by flash chromatography (20 g silica cartridge) eluting with a gradient of 0-25% DCM in heptane afforded Example 237A as a brown solid (457 mg, 1.81 mmol, 58%). ¹H NMR (400 MHz, DMSO-d₆) δ ppm: 7.51-7.46 (m, 1H), 7.45-7.40 (m, 2H), 7.30-7.24 (m, 1H), 7.18 (td, J=7.4, 1.2 Hz, 1H), 6.87 (d, J=2.2 Hz, 2H), 6.45 (t, J=2.2 Hz, 1H), 3.85-3.82 (m, 2H), 3.81 (s, 6H).

### Example 237B 2″-(3,5-dimethoxyphenyl)dispiro[[1,3]dioxolane-2,1′-cyclohexane-4′,1″-indene]

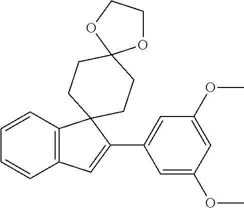 [SMILES: COc1cc(OC)cc(C2=Cc3ccccc3C23CCC2(CC3)OCCO2)c1]

Using General procedure 8a and Example 237A (344 mg, 1.36 mmol, 1 eq) as the appropriate indene, purification by flash chromatography (20 g silica cartridge) eluting with a gradient of 0-100% DCM in heptane afforded Example 237B as an orange solid (391 mg, 1.03 mmol, 76%). ¹H NMR (400 MHz, DMSO-d₆) δ ppm: 7.76-7.71 (m, 1H), 7.45-7.41 (m, 1H), 7.33-7.27 (m, 1H), 7.23-7.17 (m, 2H), 6.85 (d, J=2.2 Hz, 2H), 6.50 (t, J=2.2 Hz, 1H), 4.01-3.93 (m, 4H), 3.81 (s, 6H), 2.54-2.43 (m, 2H), 2.16 (td, J=13.6, 4.7 Hz, 2H), 1.87-1.78 (m, 2H), 1.28-1.19 (m, 2H).

### Example 237C 2′-(3,5-dimethoxyphenyl)spiro[cyclohexane-1,1′-inden]-4-one

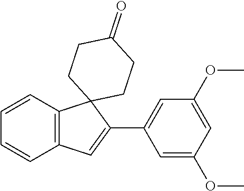 [SMILES: COc1cc(OC)cc(C2=Cc3ccccc3C23CCC(=O)CC3)c1]

To a solution of Example 237B (97.7 mg, 0.26 mmol, 1 eq) in THF (2.5 mL) was added 2 M aq. HCl solution (0.77 mL, 2 M, 1.55 mmol, 6 eq) and the mixture was heated at 100° C. for 1 h under microwave irradiation. The mixture was allowed to cool to rt and partitioned between DCM and sat. aq. NaHCO₃ solution. The organic phase was washed with brine, dried (MgSO₄) and concentrated in vacuo to afford Example 237C as an orange gum (86.3 mg, 0.26 mmol) that was used directly in the subsequent step without further purification. ¹H NMR (400 MHz, DMSO-d₆) δ ppm: 7.89-7.84 (m, 1H), 7.47-7.42 (m, 1H), 7.36-7.30 (m, 1H), 7.25-7.19 (m, 1H), 6.94 (s, 1H), 6.56 (d, J=2.3 Hz, 2H), 6.53 (t, J=2.3 Hz, 1H), 3.78 (s, 6H), 2.98-2.85 (m, 2H), 2.46-2.35 (m, 4H), 1.72-1.62 (m, 2H).

### Example 237 4-(3-bromoanilino)-2′-(3,5-dimethoxyphenyl)spiro[cyclohexane-1,1′-indene]-4-carboxylic acid, diastereoisomer 1

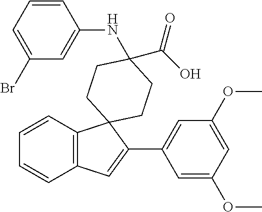 [SMILES: COc1cc(OC)cc(C2=Cc3ccccc3C23CCC(Nc2cccc(Br)c2)(C(=O)O)CC3)c1]

Using General Procedure 10 and Example 237C (86 mg, 0.26 mmol, 1 eq) as the appropriate ketone and 3-bromoaniline (0.03 mL, 0.26 mmol, 1 eq) as the appropriate aniline, a mixture of diastereoisomers was obtained. Purification by preparative HPLC at pH 4 eluting with a gradient of 67-77% MeCN in water afforded the major isomer as a single diastereoisomer, Example 237, isolated as an orange solid (1.82 mg, 0.01 mmol, 1%). LRMS calculated for C₂₉H₂₈BrNO₄: 533; found: 534 (M+H). ¹H NMR (400 MHz, DMSO-d₆) δ ppm: 7.78 (d, J=7.8 Hz, 1H), 7.43-7.39 (m, 1H), 7.32-7.27 (m, 1H), 7.21 (td, J=7.5, 1.2 Hz, 1H), 7.04-6.97 (m, 2H), 6.90-6.85 (m, 1H), 6.74-6.65 (m, 4H), 6.48 (t, J=2.2 Hz, 1H), 3.80 (s, 6H), 2.51-2.30 (m, 4H), 2.20-2.06 (m, 2H), 1.42-1.29 (m, 2H).

### Example 238

### Example 238A 2-(3,5-dimethylphenyl)-1H-indene

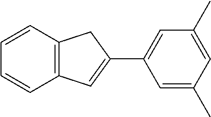 [SMILES: Cc1cc(C)cc(C2=Cc3ccccc3C2)c1]

To a solution of 1H-inden-2-ylboronic acid (500 mg, 3.13 mmol, 1 eq) in 1,4-dioxane (10 mL) and water (2.5 mL) was added Cs₂CO₃ (2.01 g, 6.16 mmol, 2 eq) and 1-bromo-3,5-dimethylbenzene (637 mg, 3.44 mmol, 1.1 eq). The mixture was sparged with N₂, followed by the addition of Pd(dppf)Cl₂×DCM (256 mg, 0.31 mmol, 0.1 eq) and then heated at 120° C. under microwave irradiation. The mixture was concentrated in vacuo and partitioned between Et₂O and sat. aq. NaHCO₃ solution. The organic phase was washed with brine, dried (MgSO₄) and concentrated in vacuo. Purification by flash chromatography (20 g silica cartridge) eluting with heptane afforded Example 238A as an off-white solid (356 mg, 1.62 mmol, 52%). ¹H NMR (400 MHz, DMSO-d₆) δ ppm: 7.50-7.45 (m, 1H), 7.43-7.39 (m, 1H), 7.37-7.32 (m, 3H), 7.29-7.23 (m, 1H), 7.19-7.14 (m, 1H), 6.95-6.92 (m, 1H), 3.84-3.80 (m, 2H), 2.33-2.30 (m, 6H).

### Example 238B 2″-(3,5-dimethylphenyl)dispiro[[1,3]dioxolane-2,1′-cyclohexane-4′,1″-indene]

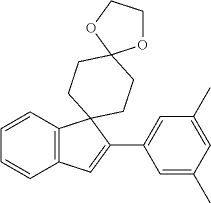 [SMILES: Cc1cc(C)cc(C2=Cc3ccccc3C23CCC2(CC3)OCCO2)c1]

Using General procedure 8a and Example 238A (300 mg, 1.36 mmol, 1 eq) as the appropriate indene, purification by flash chromatography (20 g silica cartridge) eluting with a gradient of 0-50% DCM in heptane afforded Example 238B as orange glass (375 mg, 1.08 mmol, 80%). ¹H NMR (400 MHz, DMSO-d₆) δ ppm: 7.74-7.70 (m, 1H), 7.43-7.38 (m, 1H), 7.31-7.26 (m, 1H), 7.23-7.16 (m, 3H), 7.01-6.97 (m, 2H), 3.95 (s, 4H), 2.43-2.30 (m, 8H), 2.15 (td, J=13.5, 4.7 Hz, 2H), 1.85-1.77 (m, 2H), 1.29-1.20 (m, 2H).

### Example 238C 2′-(3,5-dimethylphenyl)spiro[cyclohexane-1,1′-inden]-4-one

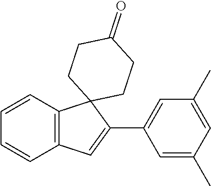 [SMILES: Cc1cc(C)cc(C2=Cc3ccccc3C23CCC(=O)CC3)c1]

To a solution of Example 238B (89.5 mg, 0.26 mmol, 1 eq) in THF (2.5 mL) was added 2 M aq. HCl solution (0.77 mL, 1.55 mmol, 6 eq) and the mixture was heated at 100° C. for 1 h under microwave irradiation. The mixture was allowed to cool to rt and partitioned between DCM and sat. aq. NaHCO₃ solution. The organic phase was washed with brine, dried (MgSO₄) and concentrated in vacuo to afford Example 238C as a cream solid (78 mg, 0.26 mmol, 100%) that was used directly in the subsequent step without further purification. ¹H NMR (400 MHz, DMSO-d₆) δ ppm: 7.90-7.86 (m, 1H), 7.46-7.41 (m, 1H), 7.35-7.30 (m, 1H), 7.21 (td, J=7.5, 1.3 Hz, 1H), 7.07-7.03 (m, 2H), 7.02-6.99 (m, 1H), 6.87 (s, 1H), 3.01-2.87 (m, 2H), 2.48-2.28 (m, 10H), 1.71-1.58 (m, 2H).

### Example 238 4-(3-bromoanilino)-2′-(3,5-dimethylphenyl)spiro[cyclohexane-1,1′-indene]-4-carboxylic acid, diastereoisomer 1

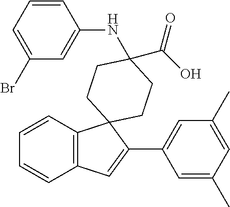 [SMILES: Cc1cc(C)cc(C2=Cc3ccccc3C23CCC(Nc2cccc(Br)c2)(C(=O)O)CC3)c1]

Using General Procedure 10 and Example 238C (78 mg, 0.26 mmol, 1 eq) as the appropriate ketone and 3-bromoaniline (0.03 mL, 0.26 mmol, 1 eq) as the appropriate aniline, a mixture of diastereoisomers was obtained. Purification by preparative HPLC at pH 4 eluting with a gradient of 65-75% MeCN in water afforded the major isomer as a single diastereoisomer, Example 238, isolated as a yellow solid (0.9 mg, 0.01 mmol, 1%). LRMS calculated for C₂₉H₂₈BrNO₂: 501; found: 502 (M+H). ¹H NMR (400 MHz, DMSO-d₆) δ ppm: 7.79 (d, J=7.6 Hz, 1H), 7.40 (dd, J=7.4, 1.3 Hz, 1H), 7.32-7.16 (m, 4H), 7.03-6.94 (m, 3H), 6.91-6.87 (m, 1H), 6.72-6.64 (m, 2H), 2.51-2.34 (m, 4H), 2.32 (s, 6H), 2.18-2.04 (m, 2H), 1.33-1.20 (m, 2H).

### Example 239

### Example 239A 2-(1H-inden-2-yl)-5-methylfuran

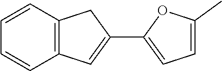

To an oven-dried microwave vial was added Example 212A (500 mg, 2.07 mmol, 1 eq), 2-bromo-5-methylfuran (0.24 mL, 2.27 mmol, 1.1 eq) and Cs₂CO₃ (1.35 g, 4.13 mmol, 2 eq) in 1,4-dioxane (10 mL) and water (2.5 mL). The mixture was sparged with N₂ followed by the addition of Pd(dppf)Cl₂×DCM (169 mg, 0.21 mmol, 0.1 eq) and heated at 120° C. for 1 h under microwave irradiation. The reaction was partitioned between EtOAc and sat. aq. NaHCO₃ solution, and the organic phase was washed with brine, dried (MgSO₄) and concentrated in vacuo. Purification by flash chromatography (25 g silica cartridge) eluting with a gradient of 0-5% EtOAc in heptane afforded Example 239A as a white powder (132 mg, 0.67 mmol, 33%). ¹H NMR (400 MHz, DMSO-d₆) δ ppm: 7.46-7.41 (m, 1H), 7.39-7.35 (m, 1H), 7.27-7.21 (m, 1H), 7.14 (td, J=7.4, 1.2 Hz, 1H), 7.01-6.97 (m, 1H), 6.57 (d, J=3.2 Hz, 1H), 6.21-6.18 (m, 1H), 3.74-3.71 (m, 2H), 2.36-2.32 (m, 3H).

### Example 239B 2″-(5-methylfuran-2-yl)dispiro[[1,3]dioxolane-2,1′-cyclohexane-4′,1″-indene]

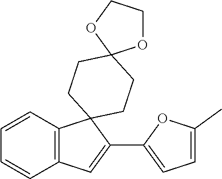 [SMILES: Cc1ccc(C2=Cc3ccccc3C23CCC2(CC3)OCCO2)o1]

Using General procedure 8a and Example 239A (132 mg, 0.67 mmol, 1 eq) as the appropriate indene Example 239B was obtained as a red solid (122 mg, 0.38 mmol, 56%). ¹H NMR (400 MHz, DMSO-d₆) δ ppm: 7.76-7.71 (m, 1H), 7.46-7.42 (m, 1H), 7.32-7.27 (m, 1H), 7.17 (td, J=7.5, 1.3 Hz, 1H), 7.02 (s, 1H), 6.53 (d, J=3.3 Hz, 1H), 6.29-6.25 (m, 1H), 4.06-3.97 (m, 4H), 2.48-2.32 (m, 5H), 2.17 (td, J=13.9, 4.7 Hz, 2H), 1.87-1.78 (m, 2H), 1.22-1.14 (m, 2H).

### Example 239 4-(3-bromoanilino)-2′-(5-methylfuran-2-yl)-2′,3′-dihydrospiro[cyclohexane-1,1′-indene]-4-carboxylic acid

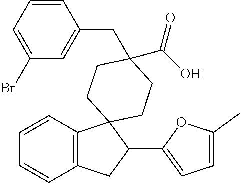 [SMILES: Cc1ccc(C2Cc3ccccc3C23CCC(Cc2cccc(Br)c2)(C(=O)O)CC3)o1]

To a solution of Example 239B (122 mg, 0.38 mmol, 1 eq) in EtOH (4 mL) and THE (4 mL) was added catalytic 10% Pd/C under N₂. The mixture was evacuated and backfilled with N₂ (×3), then evacuated and filled with H₂ and shaken at rt for 2 h under an atmosphere of H₂. The mixture was filtered through a celite pad, washed with MeOH. The combined filtrates were concentrated in vacuo to afford an intermediate which was treated according to General procedure 9 followed by General Procedure 10 using 3-bromoaniline (39 μL, 0.36 mmol, 1 eq) as the appropriate aniline. A racemic mixture of diastereoisomers was obtained. Purification using a DCM-wet PE-AX cartridge (5 g), washing with DCM, MeOH and eluting with 5% HCOOH in DCM, followed by preparative HPLC at pH 4 eluting with a gradient of 57-67% MeCN in water and then final purification using a DCM-wet PE-AX cartridge (2 g) washing with DCM, MeOH and eluting with 5% HCOOH in DCM afforded a racemic mixture of diastereoisomers, Example 239, isolated as a colourless foam (19 mg, 0.04 mmol, 11%). LRMS calculated for C₂₆H₂₆BrNO₃: 479; found: 480 (M+H). ¹H NMR (400 MHz, DMSO-d₆) δ ppm: 12.12 (br s, 1H), 7.39-7.12 (m, 4H), 7.03-6.93 (m, 1H), 6.79-6.63 (m, 2H), 6.60-6.55/6.51-6.46 (m, 1H), 6.19 (br s, 1H), 6.03-5.97 (m, 1H), 5.96-5.92 (m, 1H), 3.55-3.04 (m, 3H), 2.59-1.22 (m, 11H).

### Example 240 (1r,2′S,4S)-4-(3-chloroanilino)-2′-(4-methoxyphenyl)-2′,3′-dihydrospiro[cyclohexane-1,1′-indene]-4-carboxylic acid

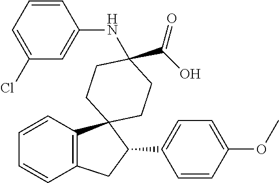 [SMILES: COc1ccc([C@@H]2Cc3ccccc3[C@]23CC[C@@](Nc2cccc(Cl)c2)(C(=O)O)CC3)cc1]

and

### Example 241 (1r,2′R,4R)-4-(3-chloroanilino)-2′-(4-methoxyphenyl)-2′,3′-dihydrospiro[cyclohexane-1,1′-indene]-4-carboxylic acid

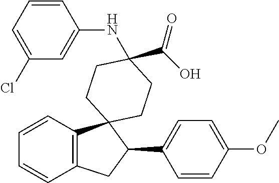 [SMILES: COc1ccc([C@H]2Cc3ccccc3[C@]23CC[C@@](Nc2cccc(Cl)c2)(C(=O)O)CC3)cc1]

Using General procedure 19 and Example 230 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 240. ¹H NMR (500 MHz, DMSO-d₆) δ ppm: 12.63 (br s, 1H), 7.31-7.17 (m, 4H), 7.01 (t, 1H), 6.96 (m, 2H), 6.77 (m, 2H), 6.52 (m, 1H), 6.52 (m, 1H), 6.45 (dm, 1H), 6.00 (br s, 1H), 3.69 (s, 3H), 3.35 (m, 1H), 3.34/2.94 (m+m, 2H), 2.42-1.23 (m, 8H). HRMS calculated for C₂₈H₂₈NO₃Cl: 461.1758; found: 462.1820 (M+H).

The enantiomer eluting later was collected as Example 241. HRMS calculated for C₂₈H₂₈NO₃Cl: 461.1758; found: 462.1853 (M+H).

### Example 242 (1r,4r)-4-(3-chloroanilino)-2′-(4-methoxy-3-methylphenyl)-2′,3′-dihydrospiro[cyclohexane-1,1′-indene]-4-carboxylic acid, enantiomer 2

and

### Example 243 (1r,4r)-4-(3-chloroanilino)-2′-(4-methoxy-3-methylphenyl)-2′,3′-dihydrospiro[cyclohexane-1,1′-indene]-4-carboxylic acid, enantiomer 1

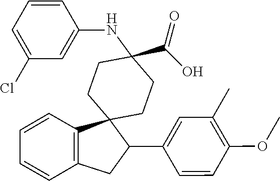

Using General procedure 19 and Example 225 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was purified further via prep RP-HPLC using 25 mM aq. NH₄HCO₃ solution and MeCN as eluents resulting Example 243. HRMS calculated for C₂₉H₃₀NO₃Cl: 475.1914; found: 476.1982 (M+H).

The enantiomer eluting later was collected as Example 242. HRMS calculated for C₂₉H₃₀NO₃Cl: 475.1914; found: 476.1984 (M+H).

### Example 244

### Example 244A 2″-[4-(benzyloxy)phenyl]dispiro[[1,3]dioxolane-2,1′-cyclohexane-4′,1″-indene]

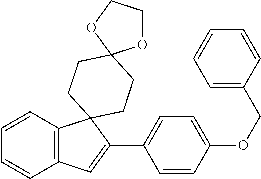 [SMILES: C1=C(c2ccc(OCc3ccccc3)cc2)C2(CCC3(CC2)OCCO3)c2ccccc21]

To an oven-dried microwave vial was added Preparation 4aA (273 mg, 0.85 mmol, 1 eq), 4-benzyloxyphenyl boronic acid (233 mg, 1.02 mmol, 1.2 eq) and Cs₂CO₃ (554 mg, 1.7 mmol, 2 eq) in 1,4-dioxane (4 mL) and water (1 mL). The mixture was sparged with N₂ followed by the addition of Pd(dppf)Cl₂×DCM (69.4 mg, 0.08 mmol, 0.1 eq) and heated at 120° C. for 2 h under microwave irradiation. The reaction was partitioned between EtOAc and sat. aq. NaHCO₃ solution, and the organic phase was washed with brine, dried (MgSO₄) and concentrated in vacuo. Purification by flash chromatography (50 g silica cartridge) eluting with a gradient of 0-10% EtOAc in heptane afforded Example 244A as a yellow powder (182 mg, 0.43 mmol, 50%). ¹H NMR (400 MHz, DMSO-d₆) δ ppm: 7.74-7.69 (m, 1H), 7.61-7.56 (m, 2H), 7.51-7.46 (m, 2H), 7.45-7.32 (m, 4H), 7.31-7.26 (m, 1H), 7.17 (td, J=7.6, 1.3 Hz, 1H), 7.11-7.06 (m, 2H), 7.02 (s, 1H), 5.17 (s, 2H), 4.00-3.94 (m, 4H), 2.45-2.35 (m, 2H), 2.22-2.11 (m, 2H), 1.86-1.77 (m, 2H), 1.28-1.19 (m, 2H).

### Example 244B 2″-[4-(benzyloxy)phenyl]-2″,3″-dihydrodispiro[[1,3]dioxolane-2,1′-cyclohexane-4′,1″-indene]

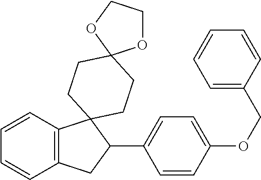 [SMILES: c1ccc(COc2ccc(C3Cc4ccccc4C34CCC3(CC4)OCCO3)cc2)cc1]

To a solution of Example 244A (99 mg, 0.29 mmol, 1 eq) in EtOH (5 mL) and THE (5 mL) was added catalytic 10% Pd/C under N₂. The mixture was evacuated and backfilled with N₂ (×3), then evacuated and filled with H₂ and shaken at rt for 2 h under an atmosphere of H₂. The mixture was filtered through a celite pad, washed with MeOH. The combined filtrates were concentrated in vacuo. Flash chromatography (5 g silica cartridge) eluting with a gradient of 0-10% EtOAc in heptane afforded a racemate, Example 244B, isolated as a colourless oil (125 mg, 0.29 mmol, 68%). LRMS calculated for C₂₉H₃₀O₃: 426; found: 427 (M+H). ¹H NMR (400 MHz, DMSO-d₆) δ ppm: 7.46-7.41 (m, 2H), 7.41-7.36 (m, 2H), 7.36-7.30 (m, 1H), 7.29-7.16 (m, 4H), 7.00-6.93 (m, 2H), 6.88-6.82 (m, 2H), 5.03 (s, 2H), 3.89-3.78 (m, 4H), 3.51-3.45 (m, 1H), 3.43-3.35 (m, 1H), 2.95-2.87 (m, 1H), 1.94-1.56 (m, 5H), 1.46-1.23 (m, 3H).

### Example 244C 2′-[4-(benzyloxy)phenyl]-2′,3′-dihydrospiro[cyclohexane-1,1′-inden]-4-one

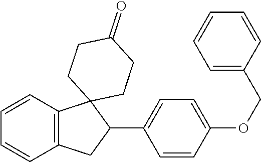 [SMILES: O=C1CCC2(CC1)c1ccccc1CC2c1ccc(OCc2ccccc2)cc1]

Using General procedure 9 and Example 244B (125 mg, 0.29 mmol, 1 eq) as the appropriate ketal afforded a racemate, Example 244C, isolated as a colourless oil (96 mg, 0.25 mmol, 86%) that was used directly in the subsequent step without further purification. LRMS calculated for C₂₇H₂₆O₂: 382; found: 383 (M+H). ¹H NMR (400 MHz, DMSO-d₆) δ ppm: 7.46-7.28 (m, 7H), 7.27-7.19 (m, 2H), 7.11-7.05 (m, 2H), 6.91-6.85 (m, 2H), 5.03 (s, 2H), 3.68 (dd, J=7.6, 4.2 Hz, 1H), 3.45 (dd, J=16.3, 7.6 Hz, 1H), 3.01 (dd, J=16.3, 4.2 Hz, 1H), 2.80-2.68 (m, 1H), 2.47-2.38 (m, 1H), 2.13-1.98 (m, 4H), 1.93-1.82 (m, 1H), 1.55-1.46 (m, 1H).

### Example 244 2′-[4-(benzyloxy)phenyl]-4-(3-bromoanilino)-2′,3′-dihydrospiro[cyclohexane-1,1′-indene]-4-carboxylic acid

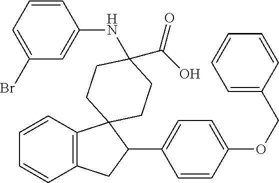 [SMILES: O=C(O)C1(Nc2cccc(Br)c2)CCC2(CC1)c1ccccc1CC2c1ccc(OCc2ccccc2)cc1]

Using General Procedure 10 and Example 244C (96 mg, 0.25 mmol, 1 eq) as the appropriate ketone and 3-bromoaniline (27 μL, 0.25 mmol, 1 eq) as the appropriate aniline, a racemic mixture of diastereoisomers was obtained. Purification using a DCM-wet PE-AX cartridge (5 g), washing with DCM, MeOH and eluting with 5% HCOOH in DCM afforded a racemic mixture of diastereoisomers, Example 244, isolated as a colourless foam (23 mg, 0.04 mmol, 16%). LRMS calculated for C₃₄H₃₂BrNO₃: 581; found: 582 (M+H). ¹H NMR (400 MHz, DMSO-d₆) δ ppm: 7.47-7.16 (m, 9H), 7.00-6.93 (m, 3H), 6.89-6.83 (m, 2H), 6.70 (t, J=1.9 Hz, 1H), 6.68-6.64 (m, 1H), 6.52-6.47 (m, 1H), 5.06-5.00 (m, 2H), 3.01-2.90 (m, 1H), 2.43-2.32 (m, 1H), 2.13-1.78 (m, 3H), 1.67-1.51 (m, 3H), 1.37-1.25 (m, 1H).

### Example 245 (1s,4s)-2′-(1-benzofuran-3-yl)-4-(3-chloroanilino)spiro[cyclohexane-1,1′-indene]-4-carboxylic acid

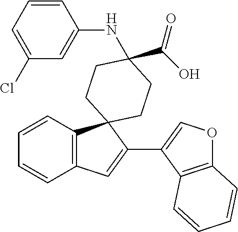 [SMILES: O=C(O)[C@]1(Nc2cccc(Cl)c2)CC[C@]2(CC1)C(c1coc3ccccc13)=Cc1ccccc12]

Using General procedure 18 and Preparation 6a as the appropriate 2-bromo-indene and benzofuran-3-ylboronic acid as the appropriate boronic acid, Example 245 was obtained. HRMS calculated for C₂₉H₂₄NO₃Cl: 469.1445; found: 470.1515 (M+H).

### Example 246 (1s,4s)-4-(3-chloroanilino)-2′-(2-chloro-5-methoxyphenyl)spiro[cyclohexane-1,1′-indene]-4-carboxylic acid

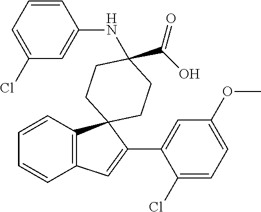 [SMILES: COc1ccc(Cl)c(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (2-chloro-5-methoxy-phenyl)boronic acid as the appropriate boronic acid, Example 246 was obtained. ¹H NMR (500 MHz, DMSO-d₆) δ ppm: 11.94 (br s, 1H), 7.74 (d, 1H), 7.44 (d, 1H), 7.41 (d, 1H), 7.31 (t, 1H), 7.24 (t, 1H), 7.00 (dd, 1H), 6.98 (t, 1H), 6.78 (d, 1H), 6.63 (s, 1H), 6.50 (dd, 1H), 6.42 (dd, 1H), 6.35 (dd, 1H), 6.06 (br s, 1H), 3.74 (s, 3H), 2.41/2.09 (t/d, 4H), 2.05/1.3 (t/d, 4H). HRMS calculated for C₂₈H₂₅NO₃Cl₂: 493.1212; found: 494.1286 (M+H).

### Example 247 (1s,4s)-2′-(1-benzofuran-5-yl)-4-(3-chloroanilino)spiro[cyclohexane-1,1′-indene]-4-carboxylic acid

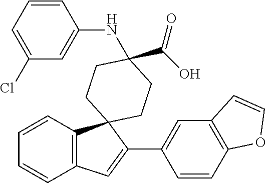 [SMILES: O=C(O)[C@]1(Nc2cccc(Cl)c2)CC[C@]2(CC1)C(c1ccc3occc3c1)=Cc1ccccc12]

Using General procedure 18 and Preparation 6a as the appropriate 2-bromo-indene and 2-(benzofuran-5-yl)-4,4,5,5-tetramethyl-1,3,2-dioxaborolane as the appropriate boronic ester, Example 247 was obtained. ¹H NMR (500 MHz, DMSO-d₆) δ ppm: 12.30 (br s, 1H), 8.08 (dd, 1H), 8.04 (d, 1H), 7.80 (t, 1H), 7.76 (dd, 1H), 7.60 (d, 1H), 7.44 (t, 1H), 7.30 (d, 1H), 7.24 (s, 1H), 7.21 (d, 1H), 7.12 (t, 1H), 6.93 (dd, 1H), 6.76 (dd, 1H), 6.65 (dd, 1H), 6.58 (dd, 1H), 6.56 (br s, 1H), 2.58/1.07 (t/d, 4H), 2.46/2.28 (t/d, 4H). HRMS calculated for C₂₉H₂₄NO₃Cl: 469.1445; found: 470.1518 (M+H).

### Example 248 (1s,4s)-4-(3-chloroanilino)-2′-(2-methoxy-6-methylphenyl)spiro[cyclohexane-1,1′-indene]-4-carboxylic acid

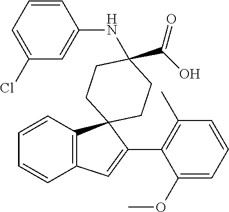 [SMILES: COc1cccc(C)c1C1=Cc2ccccc2[C@]12CC[C@@](Nc1cccc(Cl)c1)(C(=O)O)CC2]

Using General procedure 18 and Preparation 6a as the appropriate 2-bromo-indene and (2-methoxy-6-methyl-phenyl)boronic acid as the appropriate boronic acid, Example 248 was obtained. HRMS calculated for C₂₉H₂₈NO₃Cl: 473.1758; found: 474.1827 (M+H).

### Example 249 (1s,4s)-4-(3-chloroanilino)-2′-(2,6-dimethylphenyl)spiro[cyclohexane-1,1′-indene]-4-carboxylic acid

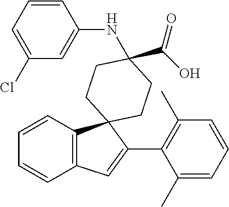 [SMILES: Cc1cccc(C)c1C1=Cc2ccccc2[C@]12CC[C@@](Nc1cccc(Cl)c1)(C(=O)O)CC2]

Using General procedure 18 and Preparation 6a as the appropriate 2-bromo-indene and (2,6-dimethylphenyl)boronic acid as the appropriate boronic acid, Example 249 was obtained. HRMS calculated for C₂₉H₂₈NO₂Cl: 457.1808; found: 458.1884 (M+H).

### Example 250 (1s,4s)-4-(3-chloroanilino)-2′-(2-chloro-6-methoxyphenyl)spiro[cyclohexane-1,1′-indene]-4-carboxylic acid

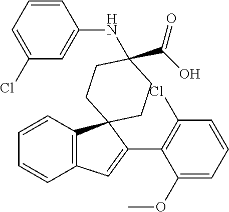 [SMILES: COc1cccc(Cl)c1C1=Cc2ccccc2[C@]12CC[C@@](Nc1cccc(Cl)c1)(C(=O)O)CC2]

Using General procedure 18 and Preparation 6a as the appropriate 2-bromo-indene and (2-chloro-6-methoxy-phenyl)boronic acid as the appropriate boronic acid, Example 250 was obtained. HRMS calculated for C₂₈H₂₅NO₃Cl₂: 493.1212; found: 494.1289 (M+H).

### Example 251 (1s,4s)-4-(3-chloroanilino)-2′-(2-chloro-4-methoxyphenyl)spiro[cyclohexane-1,1′-indene]-4-carboxylic acid

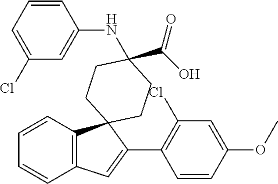 [SMILES: COc1ccc(C2=Cc3ccccc3[C@]23CC[C@@](Nc2cccc(Cl)c2)(C(=O)O)CC3)c(Cl)c1]

Using General procedure 18 and Preparation 6a as the appropriate 2-bromo-indene and (2-chloro-4-methoxy-phenyl)boronic acid as the appropriate boronic acid, Example 251 was obtained. HRMS calculated for C₂₈H₂₅NO₃Cl₂: 493.1212; found: 494.1277 (M+H).

### Example 252 (1s,4s)-4-(3-chloroanilino)-2′-(5-methoxy-2-methylphenyl)spiro[cyclohexane-1,1′-indene]-4-carboxylic acid

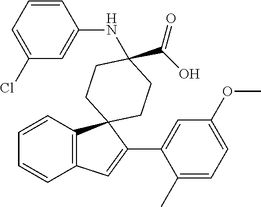 [SMILES: COc1ccc(C)c(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (5-methoxy-2-methyl-phenyl)boronic acid as the appropriate boronic acid, Example 252 was obtained. HRMS calculated for C₂₉H₂₈NO₃Cl: 473.1758; found: 474.1824 (M+H).

### Example 253 (1s,4s)-4-(3-chloroanilino)-2′-(3-methoxyphenyl)spiro[cyclohexane-1,1′-indene]-4-carboxylic acid

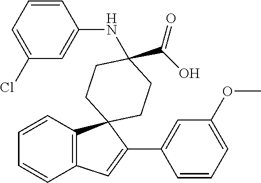 [SMILES: COc1cccc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (3-methoxyphenyl)boronic acid as the appropriate boronic acid, Example 253 was obtained. HRMS calculated for C₂₈H₂₆NO₃Cl: 459.1601; found: 460.1686 (M+H).

### Example 254 (1r,4r)-4-(3-chloroanilino)-2′-(4-methoxy-2-methylphenyl)-2′,3′-dihydrospiro[cyclohexane-1,1′-indene]-4-carboxylic acid, enantiomer 1

and

### Example 255 (1r,4r)-4-(3-chloroanilino)-2′-(4-methoxy-2-methylphenyl)-2′,3′-dihydrospiro[cyclohexane-1,1′-indene]-4-carboxylic acid, enantiomer 2

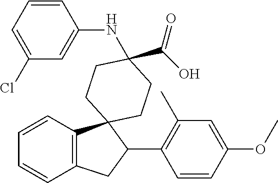

and

### Example 256 (1r,4r)-4-anilino-2′-(4-methoxy-2-methylphenyl)-2′,3′-dihydrospiro[cyclohexane-1,1′-indene]-4-carboxylic acid, enantiomer 1

and

### Example 257 (1r,4r)-4-anilino-2′-(4-methoxy-2-methylphenyl)-2′,3′-dihydrospiro[cyclohexane-1,1′-indene]-4-carboxylic acid, enantiomer 2

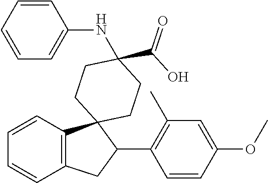

Using General procedure 19 and Example 229 as the appropriate indene and using Pd/C in EtOH/THF (1:1) instead of Pt/C in EtOAc, partial dehalogenation occurred. The mixture was separated via prep RP-HPLC using 25 mM aq. NH₄HCO₃ solution and MeCN as eluents. Both racemates were separated via chiral chromatography. Column: AD, 50×500 mm, 20 μm, eluents: EtOH+0.05% HCOOH. The enantiomer eluting earlier was collected as Example 254. HRMS calculated for C₂₉H₃₀NO₃Cl: 475.1914; found: 476.2000 (M+H).

The enantiomer eluting later was collected as Example 255. HRMS calculated for C₂₉H₃₀NO₃Cl: 475.1914; found: 476.1999 (M+H).

The enantiomer eluting earlier was collected as Example 256. HRMS calculated for C₂₉H₃₁NO₃: 441.2304; found: 442.2377 (M+H).

The enantiomer eluting later was collected as Example 257. HRMS calculated for C₂₉H₃₁NO₃: 441.2304; found: 442.2386 (M+H).

### Example 258 (1r,4r)-4-(3-chloroanilino)-2′-(2-chloro-5-methoxyphenyl)-2′,3′-dihydrospiro[cyclohexane-1,1′-indene]-4-carboxylic acid, enantiomer 1

and

### Example 259 (1r,4r)-4-(3-chloroanilino)-2′-(2-chloro-5-methoxyphenyl)-2′,3′-dihydrospiro[cyclohexane-1,1′-indene]-4-carboxylic acid, enantiomer 2

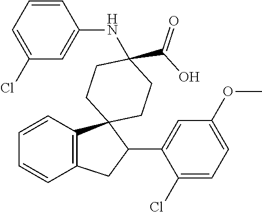

and

### Example 260 (1r,4r)-4-anilino-2′-(2-chloro-5-methoxyphenyl)-2′,3′-dihydrospiro[cyclohexane-1,1′-indene]-4-carboxylic acid, enantiomer 1

### Example 261 (1r,4r)-4-anilino-2′-(2-chloro-5-methoxyphenyl)-2′,3′-dihydrospiro[cyclohexane-1,1′-indene]-4-carboxylic acid, enantiomer 2

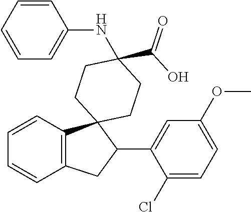

Using General procedure 19 and Example 246 as the appropriate indene and using Pd/C in EtOH/THF (1:1) instead of Pt/C in EtOAc, partial dehalogenation occurred. The mixture was separated via prep RP-HPLC using 25 mM aq. NH₄HCO₃ solution and MeCN as eluents. Both racemates were separated via chiral chromatography. Column: AD, 50×500 mm, 20 μm, eluents: EtOH+0.05% HCOOH. The enantiomer eluting earlier was collected as Example 258. HRMS calculated for C₂₈H₂₇NO₃Cl₂: 495.1368; found: 496.1458 (M+H).

The enantiomer eluting later was collected as Example 259. HRMS calculated for C₂₈H₂₇NO₃Cl₂: 495.1368; found: 496.1460 (M+H).

The enantiomer eluting earlier was collected as Example 260. ¹H NMR (500 MHz, DMSO-d₆) δ ppm: 7.32 (d, 1H), 7.31-7.19 (m, 4H), 7.00 (m, 2H), 6.80 (dd, 1H), 6.57 (m, 2H), 6.51 (m, 1H), 6.30 (d, 1H), 3.99 (dd, 1H), 3.51 (s, 3H), 3.49/2.86 (dd+dd, 2H), 2.40-1.40 (m, 8H). HRMS calculated for C₂₈H₂₈NO₃Cl: 461.1758; found: 462.1833 (M+H).

The enantiomer eluting later was collected as Example 261. HRMS calculated for C₂₈H₂₈NO₃Cl: 461.1758; found: 462.1839 (M+H).

### Example 262 and Example 263 Example 262A 2-(1H-inden-2-yl)-5-methylthiophene

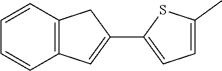

To a solution of 1H-indene-2-boronic acid (500 mg, 3.13 mmol, 1 eq) in 1,4-dioxane (10 mL) and water (2.5 mL) was added Cs₂CO₃ (2.01 g, 6.16 mmol, 2 eq) and 2-bromo-5-methylthiophene (0.39 mL, 3.44 mmol, 1.1 eq). The mixture was sparged with N₂ followed by the addition of Pd(dppf)Cl₂×DCM (256 mg, 0.31 mmol, 0.1 eq) and heated at 120° C. for 1 h under microwave irradiation. The mixture was concentrated in vacuo and partitioned between Et₂O and sat. aq. NaHCO₃ solution. The organic phase was washed with brine, dried (MgSO₄) and concentrated in vacuo. Purification by flash chromatography (20 g silica cartridge) eluting with a gradient of 0-5% EtOAc in heptane followed by trituration with Et₂O afforded Example 262A, isolated as a white powder (183 mg, 0.86 mmol, 28%). ¹H NMR (400 MHz, DMSO-d₆) δ ppm: 7.46-7.41 (m, 1H), 7.38-7.34 (m, 1H), 7.26-7.21 (m, 1H), 7.17-7.10 (m, 2H), 6.99-6.96 (m, 1H), 6.80-6.77 (m, 1H), 3.82-3.78 (m, 2H), 2.46 (d, J=1.2 Hz, 3H).

### Example 262B 2″-(5-methylthiophen-2-yl)dispiro[[1,3]dioxolane-2,1′-cyclohexane-4′,1″-indene]

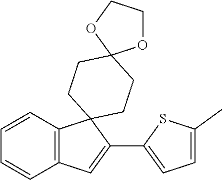 [SMILES: Cc1ccc(C2=Cc3ccccc3C23CCC2(CC3)OCCO2)s1]

Using General procedure 8a and Example 262A (183 mg, 0.86 mmol, 1 eq) as the appropriate indene, Example 262B was obtained as a purple oil (213 mg, 0.63 mmol, 73%). ¹H NMR (400 MHz, DMSO-d₆) δ ppm: 7.76-7.71 (m, 1H), 7.45-7.40 (m, 1H), 7.32-7.26 (m, 1H), 7.22-7.14 (m, 2H), 7.00 (s, 1H), 6.87-6.84 (m, 1H), 4.06-3.97 (m, 4H), 2.59-2.45 (m, 5H), 2.19 (td, J=13.8, 4.6 Hz, 2H), 1.89-1.80 (m, 2H), 1.23-1.14 (m, 2H).

### Example 262C 2″-(5-methylthiophen-2-yl)-2″,3″-dihydrodispiro[[1,3]dioxolane-2,1′-cyclohexane-4′,1″-indene]

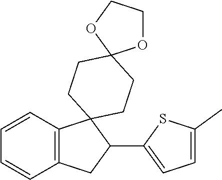 [SMILES: Cc1ccc(C2Cc3ccccc3C23CCC2(CC3)OCCO2)s1]

To a solution of Example 262B (213 mg, 0.63 mmol, 1 eq) in EtOH (5 mL) and THE (5 mL) was added catalytic 10% Pd/C under N₂. The mixture was evacuated and backfilled with N₂ (×3), then evacuated and filled with H₂ and shaken at rt under an atmosphere of H₂ until complete conversion had occurred. The mixture was filtered through a celite pad, washed with EtOH. The combined filtrates were concentrated in vacuo to afford a racemate, Example 262C, isolated as a grey solid (187 mg, 0.55 mmol, 87%) that was used directly in the subsequent step without further purification. ¹H NMR (400 MHz, DMSO-d₆) δ ppm: 7.30-7.16 (m, 4H), 6.69 (d, J=3.3 Hz, 1H), 6.55-6.52 (m, 1H), 3.92-3.80 (m, 5H), 3.44-3.36 (m, 1H), 2.84 (dd, J=15.9, 3.1 Hz, 1H), 2.28 (d, J=1.1 Hz, 3H), 1.93-1.73 (m, 4H), 1.60-1.36 (m, 4H).

### Example 262 4-(3-bromoanilino)-2′-(5-methylthiophen-2-yl)-2′,3′-dihydrospiro[cyclohexane-1,1′-indene]-4-carboxylic acid (mixture of 3 stereoisomers)

and

### Example 263 4-(3-bromoanilino)-2′-(5-methylthiophen-2-yl)-2′,3′-dihydrospiro[cyclohexane-1,1′-indene]-4-carboxylic acid, diastereoisomer 2 enantiomer 2

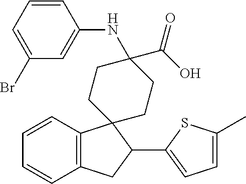 [SMILES: Cc1ccc(C2Cc3ccccc3C23CCC(Nc2cccc(Br)c2)(C(=O)O)CC3)s1]

Using General procedure 9 and Example 262C (187 mg, 0.55 mmol, 1 eq) as the appropriate ketal, afforded an intermediate which was treated according to General Procedure 10 using 3-bromoaniline (59 μL, 0.54 mmol, 1 eq) as the appropriate aniline, to obtain a racemic mixture of diastereoisomers, isolated as a colourless foam (38 mg, 0.08 mmol, 14%). The stereoisomers were partially separated by chiral chromatography. Column: AD, 50×500 mm, 20 μm. Eluent: 50:50 MeOH/EtOH+0.05% HCOOH. The stereoisomer mixture eluting earlier was collected as Example 262 (mixture of 3 stereoisomers). HRMS calculated for C₂₆H₂₆NO₂SBr: 495.0868; found: 496.0947 (M+H).

The single stereoisomer eluting last was collected as Example 263. ¹H NMR (500 MHz, DMSO-d₆) δ ppm: 7.33-7.15 (m, 4H), 6.95 (t, 1H), 6.73 (t, 1H), 6.68 (d, 1H), 6.64 (d, 1H), 6.55 (m, 1H), 6.52 (dd, 1H), 3.65 (t, 1H), 3.35/2.90 (dd+dd, 2H), 2.36/2.07/1.92/1.75 (m+m/m+m, 4H), 2.30 (d, 3H), 1.93/1.59/1.52/1.42 (m+m/m+m, 4H). HRMS calculated for C₂₆H₂₆NO₂SBr: 495.0868; found: 496.0952 (M+H).

### Example 264 (1s,4s)-4-(3-chloroanilino)-2′-(1-methyl-1H-indol-5-yl)spiro[cyclohexane-1,1′-indene]-4-carboxylic acid

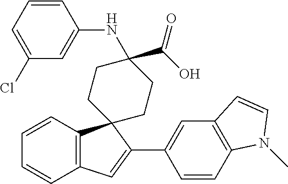 [SMILES: Cn1ccc2cc(C3=Cc4ccccc4[C@]34CC[C@@](Nc3cccc(Cl)c3)(C(=O)O)CC4)ccc21]

To an oven-dried flask was added 1-methyl-5-(4,4,5,5-tetramethyl-1,3,2-dioxaborolan-2-yl)-1H-indole (65 mg, 0.25 mmol, 1 eq), Preparation 6a (86 mg, 0.20 mmol, 0.79 eq) and K₃PO₄ (127 mg, 0.60 mmol, 2.4 eq) in 1,4-dioxane (2 mL) and water (0.4 mL). The mixture was sparged with N₂ followed by the addition of Pd(dppf)Cl₂×DCM (5 mol %, 8 mg). The mixture was heated at 120° C. for 1 h under microwave irradiation. The mixture was partitioned between EtOAc and water and the pH was adjusted to 4 with 1 M aq. HCl solution and the organic phase was washed with brine, dried (MgSO₄) and concentrated in vacuo. Purification by automated flash chromatography (CombiFlash Rf, 12 g RediSep™ silica cartridge) eluting with a gradient of 0-30% EtOAc in heptane afforded Example 264 as a white solid (35 mg, 0.07 mmol, 36%). LRMS calculated for C₃₀H₂₇ClN₂O₂: 482.18; found: 483.20 (M+H). ¹H NMR (400 MHz, DMSO-d₆) δ ppm: 12.78 (br s, 1H), 8.05 (d, J=1.9 Hz, 1H), 7.79 (d, J=7.6 Hz, 1H), 7.65 (dd, J=8.7, 1.7 Hz, 1H), 7.47-7.40 (m, 2H), 7.35 (d, J=3.0 Hz, 1H), 7.30 (td, J=7.5, 0.9 Hz, 1H), 7.22-7.12 (m, 3H), 6.83 (t, J=2.1 Hz, 1H), 6.70-6.66 (m, 1H), 6.63 (br s, 1H), 6.62-6.58 (m, 1H), 6.48 (dd, J=3.1, 0.8 Hz, 1H), 3.81 (s, 3H), 2.75-2.62 (m, 2H), 2.54-2.43 (m, 2H), 2.37-2.26 (m, 2H), 1.11-1.03 (m, 2H).

### Example 265 (1s,4s)-2′-(1-benzofuran-6-yl)-4-(3-chloroanilino)spiro[cyclohexane-1,1′-indene]-4-carboxylic acid

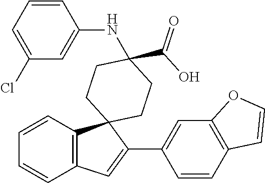 [SMILES: O=C(O)[C@]1(Nc2cccc(Cl)c2)CC[C@]2(CC1)C(c1ccc3ccoc3c1)=Cc1ccccc12]

Using General procedure 18 and Preparation 6a as the appropriate 2-bromo-indene and benzofuran-6-ylboronic acid as the appropriate boronic acid, Example 265 was obtained. ¹H NMR (500 MHz, DMSO-d₆) δ ppm: 11.93 (br s, 1H), 8.09 (m, 1H), 8.04 (d, 1H), 7.81 (dm, 1H), 7.77 (dd, 1H), 7.64 (d, 1H), 7.45 (dm, 1H), 7.37 (s, 1H), 7.31 (m, 1H), 7.22 (m, 1H), 7.11 (t, 1H), 6.98 (dd, 1H), 6.84 (t, 1H), 6.70 (dm, 1H), 6.63 (br s, 1H), 6.58 (dm, 1H), 2.60/1.05 (m+m, 4H), 2.46/2.30 (m+m, 4H). HRMS calculated for C₂₉H₂₄NO₃Cl: 469.1445; found: 470.1508 (M+H).

### Example 266 (1s,4s)-2′-(1-benzofuran-7-yl)-4-(3-chloroanilino)spiro[cyclohexane-1,1′-indene]-4-carboxylic acid

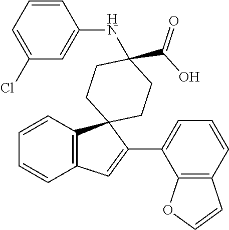 [SMILES: O=C(O)[C@]1(Nc2cccc(Cl)c2)CC[C@]2(CC1)C(c1cccc3ccoc13)=Cc1ccccc12]

Using General procedure 18 and Preparation 6a as the appropriate 2-bromo-indene and benzofuran-7-ylboronic acid as the appropriate boronic acid, Example 266 was obtained. ¹H NMR (500 MHz, DMSO-d₆) δ ppm: 12.50 (br s, 1H), 8.08 (d, 1H), 7.84 (dd, 1H), 7.84 (dm, 1H), 7.76 (s, 1H), 7.65 (dd, 1H), 7.54 (dm, 1H), 7.34 (m, 1H), 7.29 (t, 1H), 7.27 (m, 1H), 7.12 (t, 1H), 7.03 (d, 1H), 6.70 (t, 1H), 6.64 (dm, 1H), 6.58 (dm, 1H), 6.56 (br s, 1H), 2.59/1.11 (m+m, 4H), 2.47/2.29 (m+m, 4H). HRMS calculated for C₂₉H₂₄NO₃Cl: 469.1445; found: 470.1513 (M+H).

### Example 267 (1s,4s)-4-(3-chloroanilino)-2′-(2-chloro-3-methoxyphenyl)spiro[cyclohexane-1,1′-indene]-4-carboxylic acid

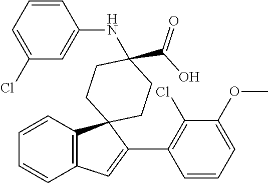 [SMILES: COc1cccc(C2=Cc3ccccc3[C@]23CC[C@@](Nc2cccc(Cl)c2)(C(=O)O)CC3)c1Cl]

Using General procedure 18 and Preparation 6a as the appropriate 2-bromo-indene and (2-chloro-3-methoxy-phenyl)boronic acid as the appropriate boronic acid, Example 267 was obtained. ¹H NMR (500 MHz, DMSO-d₆) δ ppm: 9.12 (br s, 1H), 7.76 (dm, 1H), 7.40 (dm, 1H), 7.31 (t, 1H), 7.30 (m, 1H), 7.23 (m, 1H), 7.15 (dd, 1H), 6.95 (t, 1H), 6.83 (dd, 1H), 6.59 (s, 1H), 6.46 (dm, 1H), 6.41 (t, 1H), 6.34 (dm, 1H), 5.97 (br s, 1H), 3.88 (s, 3H), 2.42/2.05 (m+m, 4H), 2.04/1.29 (m+m, 4H). HRMS calculated for C₂₈H₂₅NO₃Cl₂: 493.1212; found: 494.1306 (M+H).

### Example 268 (1r,4r)-4-(3-chloroanilino)-2′-(2-chloro-4-methoxyphenyl)-2′,3′-dihydrospiro[cyclohexane-1,1′-indene]-4-carboxylic acid, enantiomer 1

and

### Example 269 (1r,4r)-4-(3-chloroanilino)-2′-(2-chloro-4-methoxyphenyl)-2′,3′-dihydrospiro[cyclohexane-1,1′-indene]-4-carboxylic acid, enantiomer 2

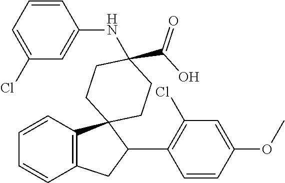

Using General procedure 19 and Example 251 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 268. ¹H NMR (500 MHz, DMSO-d₆) δ ppm: 12.67 (br s, 1H), 7.30-7.18 (m, 4H), 7.02 (t, 1H), 6.97 (d, 1H), 6.74 (m, 1H), 6.73 (m, 1H), 6.57 (t, 1H), 6.52 (dm, 1H), 6.50 (dm, 1H), 6.15 (br s, 1H), 3.97 (dd, 1H), 3.70 (s, 3H), 3.47/2.84 (dd+dd, 2H), 2.41-1.34 (m, 8H). HRMS calculated for C₂₈H₂₇NO₃Cl₂: 495.1368; found: 496.1448 (M+H).

The enantiomer eluting later was collected as Example 269. HRMS calculated for C₂₈H₂₇NO₃Cl₂: 495.1368; found: 496.1437 (M+H).

### Example 270 (1r,4r)-4-(3-chloroanilino)-2′-(5-methoxy-2-methylphenyl)-2′,3′-dihydrospiro[cyclohexane-1,1′-indene]-4-carboxylic acid, enantiomer 1

and

### Example 271 (1r,4r)-4-(3-chloroanilino)-2′-(5-methoxy-2-methylphenyl)-2′,3′-dihydrospiro[cyclohexane-1,1′-indene]-4-carboxylic acid, enantiomer 2

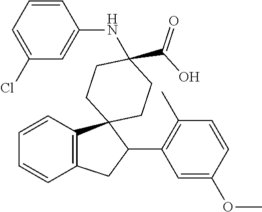

Using General procedure 19 and Example 252 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 270. ¹H NMR (500 MHz, DMSO-d₆) δ ppm: 12.66 (br s, 1H), 7.32-7.16 (m, 4H), 7.06 (d, 1H), 7.02 (t, 1H), 6.64 (dd, 1H), 6.57 (t, 1H), 6.53 (dm, 1H), 6.50 (dm, 1H), 6.19 (br s, 1H), 6.18 (d, 1H), 3.65 (dd, 1H), 3.47 (s, 3H), 3.46/2.79 (dd+dd, 2H), 2.40-1.38 (m, 8H), 2.33 (s, 3H). HRMS calculated for C₂₉H₃₀NO₃Cl: 475.1914; found: 476.1976 (M+H).

The enantiomer eluting later was collected as Example 271. HRMS calculated for C₂₉H₃₀NO₃Cl: 475.1914; found: 476.1988 (M+H).

### Example 272 (1r,4r)-4-(3-chloroanilino)-2′-(3-methoxyphenyl)-2′,3′-dihydrospiro[cyclohexane-1,1′-indene]-4-carboxylic acid, enantiomer 1

and

### Example 273 (1r,4r)-4-(3-chloroanilino)-2′-(3-methoxyphenyl)-2′,3′-dihydrospiro[cyclohexane-1,1′-indene]-4-carboxylic acid, enantiomer 2

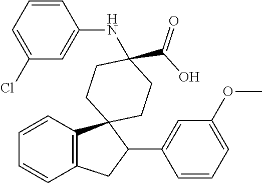

Using General procedure 19 and Example 253 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 272. ¹H NMR (500 MHz, DMSO-d₆) δ ppm: 12.64 (br s, 1H), 7.32-7.16 (m, 4H), 7.11 (t, 1H), 7.02 (t, 1H), 6.75 (dm, 1H), 6.60 (dm, 1H), 6.58 (br s, 1H), 6.53 (m, 1H), 6.52 (m, 1H), 6.46 (dm, 1H), 6.04 (br s, 1H), 3.65 (s, 3H), 3.38 (m, 1H), 3.37/2.96 (m+m, 2H), 2.43-1.28 (m, 8H). HRMS calculated for C₂₈H₂₈NO₃Cl: 461.1758; found: 462.1834 (M+H).

The enantiomer eluting later was collected as Example 273. HRMS calculated for C₂₈H₂₈NO₃Cl: 461.1758; found: 462.1826 (M+H).

### Example 274 (1r,4r)-4-(3-chloroanilino)-2′-(1-methyl-1H-indol-5-yl)-2′,3′-dihydrospiro[cyclohexane-1,1′-indene]-4-carboxylic acid

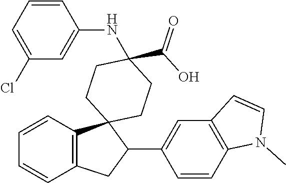

To a solution of Example 264 (35 mg, 0.59 mmol, 1 eq) in EtOH (3 mL) and THF (3 mL) was added 5% Pt/C (50 mg) under N₂. The mixture was evacuated and backfilled with N₂ (×3), then evacuated and filled with H₂ and shaken at rt for 16 h under an atmosphere of H₂. The mixture was filtered through a celite pad, washed with EtOAc. The combined filtrates were concentrated in vacuo. Purification by automated flash chromatography (CombiFlash Rf, 4 g RediSep™ silica cartridge) eluting with a gradient of 0-30% EtOAc in heptane afforded a racemate, Example 274, isolated as a pale yellow solid (12 mg, 0.02 mmol, 34%). LRMS calculated for C₃₀H₂₉ClN₂O₂: 484; found: 485 (M+H). ¹H NMR (400 MHz, DMSO-d₆) δ ppm: 7.34-7.18 (m, 7H), 6.99 (t, J=8.0 Hz, 1H), 6.83-6.77 (m, 1H), 6.55-6.49 (m, 2H), 6.48-6.43 (m, 1H), 6.28 (dd, J=3.0, 0.8 Hz, 1H), 3.73 (s, 3H), 3.55-3.49 (m, 1H), 3.49-3.40 (m, 1H), 3.05-2.96 (m, 1H), 2.46-2.36 (m, 1H), 2.08-1.82 (m, 3H), 1.68-1.55 (m, 3H), 1.41-1.32 (m, 1H).

### Example 275 (1r,4r)-4-(3-chloroanilino)-2′-(cyclohex-1-en-1-yl)spiro[cyclohexane-1,1′-indene]-4-carboxylic acid

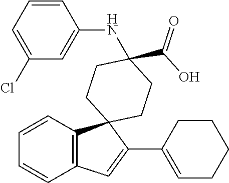 [SMILES: O=C(O)[C@]1(Nc2cccc(Cl)c2)CC[C@]2(CC1)C(C1=CCCCC1)=Cc1ccccc12]

Using General procedure 18 and Preparation 6a as the appropriate 2-bromo-indene and 2-(cyclohexen-1-yl)-4,4,5,5-tetramethyl-1,3,2-dioxaborolane as the appropriate boronic ester, Example 275 was obtained. ¹H NMR (500 MHz, DMSO-d₆) δ ppm: 12.82 (br s, 1H), 7.72 (dm, 1H), 7.36 (dm, 1H), 7.24 (m, 1H), 7.15 (m, 1H), 7.11 (t, 1H), 6.71 (s, 1H), 6.65 (t, 1H), 6.59 (dm, 1H), 6.57 (dm, 1H), 6.51 (br s, 1H), 6.42 (m, 1H), 2.54-0.83 (m, 16H). HRMS calculated for C₂₇H₂₈NO₂Cl: 433.1808; found: 434.1896 (M+H).

### Example 276 (1s,4S)-4-(3-chloroanilino)-2′-[(1R,4R)-1,7,7-trimethylbicyclo[2.2.1]hept-2-en-2-yl]spiro[cyclohexane-1,1′-indene]-4-carboxylic acid

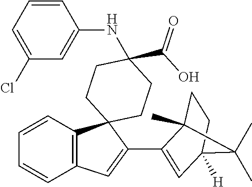 [SMILES: CC1(C)[C@H]2C=C(C3=Cc4ccccc4[C@]34CC[C@@](Nc3cccc(Cl)c3)(C(=O)O)CC4)[C@]1(C)CC2]

Using General procedure 18 and Preparation 6a as the appropriate 2-bromo-indene and [(1S,4R)-1,7,7-trimethyl-2-bicyclo[2.2.1]hept-2-enyl]boronic acid as the appropriate boronic acid, Example 276 was obtained. ¹H NMR (500 MHz, DMSO-d₆) δ ppm: 12.78 (br s, 1H), 7.71 (dm, 1H), 7.36 (dm, 1H), 7.24 (m, 1H), 7.14 (m, 1H), 7.09 (t, 1H), 6.74 (s, 1H), 6.60 (dm, 1H), 6.57 (dm, 1H), 6.55 (dm, 1H), 6.43 (m, 1H), 6.39 (br s, 1H), 2.46-0.80 (m, 8H), 2.41 (m, 1H), 1.89/1.00 (m+m, 2H), 1.55/1.12 (m+m, 2H), 1.14 (s, 3H), 0.78 (s, 6H). HRMS calculated for C₃₁H₃₄NO₂Cl: 487.2278; found: 488.2358 (M+H).

### Example 277 (1r,4r)-4-(3-chloroanilino)-2′-(cyclopent-1-en-1-yl)spiro[cyclohexane-1,1′-indene]-4-carboxylic acid

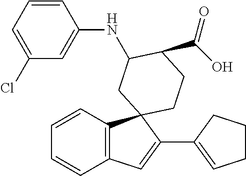 [SMILES: O=C(O)[C@H]1CC[C@@]2(CC1Nc1cccc(Cl)c1)C(C1=CCCC1)=Cc1ccccc12]

Using General procedure 18 and Preparation 6a as the appropriate 2-bromo-indene and cyclopenten-1-ylboronic acid as the appropriate boronic acid, Example 277 was obtained. ¹H NMR (500 MHz, DMSO-d₆) δ ppm: 12.80 (br s, 1H), 7.74 (dm, 1H), 7.40 (dm, 1H), 7.27 (m, 1H), 7.18 (m, 1H), 7.11 (t, 1H), 6.65 (t, 1H), 6.65 (s, 1H), 6.61 (dm, 1H), 6.58 (dm, 1H), 6.25 (br s, 1H), 6.25 (m, 1H), 2.68-0.84 (m, 14H). HRMS calculated for C₂₆H₂₆NO₂Cl: 419.1652; found: 420.1733 (M+H).

### Example 278 (1s,4s)-4-(3-chloroanilino)-2′-(5,5-dimethylcyclopent-1-en-1-yl)spiro[cyclohexane-1,1′-indene]-4-carboxylic acid

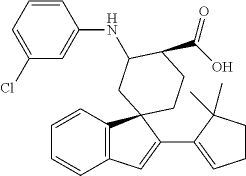 [SMILES: CC1(C)CCC=C1C1=Cc2ccccc2[C@@]12CC[C@H](C(=O)O)C(Nc1cccc(Cl)c1)C2]

Using General procedure 18 and Preparation 6a as the appropriate 2-bromo-indene and 2-(5,5-dimethylcyclopenten-1-yl)-4,4,5,5-tetramethyl-1,3,2-dioxaborolane as the appropriate boronic ester, Example 278 was obtained. ¹H NMR (500 MHz, DMSO-d₆) δ ppm: 12.79 (br s, 1H), 7.73 (dm, 1H), 7.42 (dm, 1H), 7.26 (m, 1H), 7.16 (m, 1H), 7.11 (t, 1H), 6.91 (s, 1H), 6.64 (t, 1H), 6.59 (dm, 1H), 6.58 (dm, 1H), 6.45 (s, 1H), 6.25 (m, 1H), 2.58-0.78 (m, 8H), 2.4 (m, 2H), 1.72 (t, 2H), 1.25 (s, 6H). HRMS calculated for C₂₈H₃₀NO₂Cl: 447.1965; found: 448.2050 (M+H).

### Example 279 (1r,4r)-4-(3-chloroanilino)-2′-(3-chloro-4-methoxyphenyl)-2′,3′-dihydrospiro[cyclohexane-1,1′-indene]-4-carboxylic acid, enantiomer 1

and

### Example 280 (1r,4r)-4-(3-chloroanilino)-2′-(3-chloro-4-methoxyphenyl)-2′,3′-dihydrospiro[cyclohexane-1,1′-indene]-4-carboxylic acid, enantiomer 2

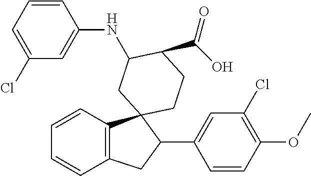

Using General procedure 19 and Example 226 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 279. ¹H NMR (500 MHz, DMSO-d₆) δ ppm: 12.65 (br s, 1H), 7.34-7.16 (m, 4H), 7.06 (s, 1H), 7.01 (t, 1H), 6.98 (s, 1H), 6.98 (s, 1H), 6.52 (m, 1H), 6.52 (dm, 1H), 6.45 (dm, 1H), 6.04 (br s, 1H), 3.79 (s, 3H), 3.38 (dd, 1H), 3.34/2.95 (dd+dd, 2H), 2.42-1.23 (m, 8H). HRMS calculated for C₂₈H₂₇NO₃Cl₂: 495.1368; found: 496.1436 (M+H).

The enantiomer eluting later was collected as Example 280. HRMS calculated for C₂₈H₂₇NO₃Cl₂: 495.1368; found: 496.1432 (M+H).

### Example 281 (1r,2′S,4S)-2′-(1-benzofuran-5-yl)-4-(3-chloroanilino)-2′,3′-dihydrospiro[cyclohexane-1,1′-indene]-4-carboxylic acid

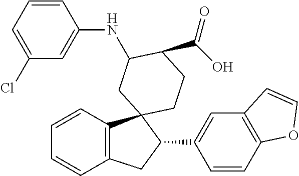 [SMILES: O=C(O)[C@H]1CC[C@]2(CC1Nc1cccc(Cl)c1)c1ccccc1C[C@H]2c1ccc2occc2c1]

and

### Example 282 (1r,2′R,4R)-2′-(1-benzofuran-5-yl)-4-(3-chloroanilino)-2′,3′-dihydrospiro[cyclohexane-1,1′-indene]-4-carboxylic acid

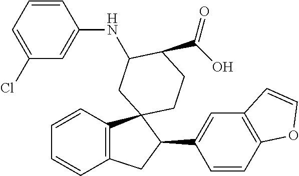 [SMILES: O=C(O)[C@H]1CC[C@]2(CC1Nc1cccc(Cl)c1)c1ccccc1C[C@@H]2c1ccc2occc2c1]

Using General procedure 19 and Example 247 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 281. HRMS calculated for C₂₉H₂₆NO₃Cl: 471.1601; found: 472.1667 (M+H).

The enantiomer eluting later was collected as Example 282. HRMS calculated for C₂₉H₂₆NO₃Cl: 471.1601; found: 472.1664 (M+H).

### Example 283 (1r,4r)-4-(3-chloroanilino)-2′-(2-chloro-6-methoxyphenyl)-2′,3′-dihydrospiro[cyclohexane-1,1′-indene]-4-carboxylic acid

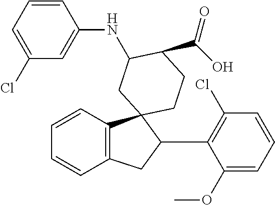

Using General procedure 19 and Example 250 as the appropriate indene and using Pd/C in EtOH/THF (1:1) instead of Pt/C in EtOAc, Example 283 was obtained as a racemate. HRMS calculated for C₂₈H₂₇NO₃Cl₂: 495.1368; found: 496.1437 (M+H).

### Example 284 (1r,4r)-2′-(1-benzofuran-3-yl)-4-(3-chloroanilino)-2′,3′-dihydrospiro[cyclohexane-1,1′-indene]-4-carboxylic acid, enantiomer 1

and

### Example 285 (1r,4r)-2′-(1-benzofuran-3-yl)-4-(3-chloroanilino)-2′,3′-dihydrospiro[cyclohexane-1,1′-indene]-4-carboxylic acid, enantiomer 2

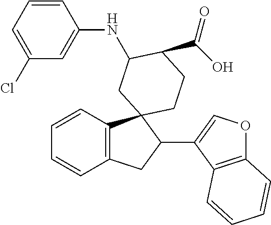

Using General procedure 19 and Example 245 as the appropriate indene and using Pd/C in EtOH/THF (1:1) instead of Pt/C in EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 284. ¹H NMR (500 MHz, DMSO-d₆) δ ppm: 12.62 (br s, 1H), 7.82 (s, 1H), 7.50 (dm, 1H), 7.38-7.23 (m, 4H), 7.20 (m, 1H), 6.98 (t, 1H), 6.97 (m, 1H), 6.65 (br d, 1H), 6.50 (m, 1H), 6.50 (m, 1H), 6.42 (dm, 1H), 6.03 (br s, 1H), 3.72 (dd, 1H), 3.41/2.98 (dd+dd, 2H), 2.48-1.41 (m, 8H). HRMS calculated for C₂₉H₂₆NO₃Cl: 471.1601; found: 472.1683 (M+H).

The enantiomer eluting later was collected as Example 285. HRMS calculated for C₂₉H₂₆NO₃Cl: 471.1601; found: 472.1667 (M+H).

### Example 286 (1r,4r)-4-(3-chloroanilino)-2′-(2-methoxy-6-methylphenyl)-2′,3′-dihydrospiro[cyclohexane-1,1′-indene]-4-carboxylic acid

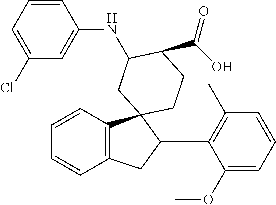

Using General procedure 19 and Example 248 as the appropriate indene and using EtOH/THF (1:1) instead of EtOAc, Example 286 was obtained as a racemate. HRMS calculated for C₂₉H₃₀NO₃Cl: 475.1914; found: 476.1986 (M+H).

### Example 287 (1r,4r)-4-(3-chloroanilino)-2′-(2,3-dihydro-1-benzofuran-6-yl)-2′,3′-dihydrospiro[cyclohexane-1,1′-indene]-4-carboxylic acid, enantiomer 1

and

### Example 288 (1r,4r)-4-(3-chloroanilino)-2′-(2,3-dihydro-1-benzofuran-6-yl)-2′,3′-dihydrospiro[cyclohexane-1,1′-indene]-4-carboxylic acid, enantiomer 2

and

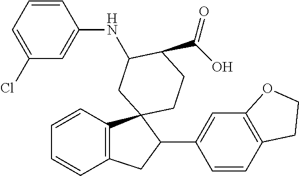

Example 289 (1r,4r)-2′-(1-benzofuran-6-yl)-4-(3-chloroanilino)-2′,3′-dihydrospiro[cyclohexane-1,1′-indene]-4-carboxylic acid, enantiomer 1

and

### Example 290 (1r,4r)-2′-(1-benzofuran-6-yl)-4-(3-chloroanilino)-2′,3′-dihydrospiro[cyclohexane-1,1′-indene]-4-carboxylic acid, enantiomer 2

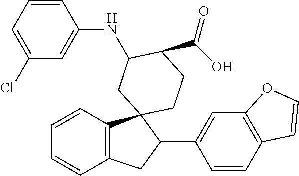

Using General procedure 19 and Example 265 as the appropriate indene and using AcOH instead of EtOAc, a partial saturation of the furan ring occurred. The mixture was separated via prep RP-HPLC using 25 mM aq. NH₄HCO₃ solution and MeCN as eluents. Both racemates were separated via chiral chromatography. Column: AD, 50×500 mm, 20 μm, eluents: EtOH+0.05% HCOOH. The enantiomer eluting earlier was collected as Example 287. HRMS calculated for C₂₉H₂₈NO₃Cl: 473.1758; found: 474.1836 (M+H).

The enantiomer eluting later was collected as Example 288. HRMS calculated for C₂₉H₂₈NO₃Cl: 473.1758; found: 474.1816 (M+H).

The enantiomer eluting earlier was collected as Example 289. HRMS calculated for C₂₉H₂₆NO₃Cl: 471.1601; found: 472.1661 (M+H).

The enantiomer eluting later was collected as Example 290. HRMS calculated for C₂₉H₂₆NO₃Cl: 471.1601; found: 472.1678 (M+H).

### Example 291 (1r,4r)-4-(3-chloroanilino)-2′-(2-chloro-3-methoxyphenyl)-2′,3′-dihydrospiro[cyclohexane-1,1′-indene]-4-carboxylic acid, enantiomer 1

and

### Example 292 (1r,4r)-4-(3-chloroanilino)-2′-(2-chloro-3-methoxyphenyl)-2′,3′-dihydrospiro[cyclohexane-1,1′-indene]-4-carboxylic acid, enantiomer 2

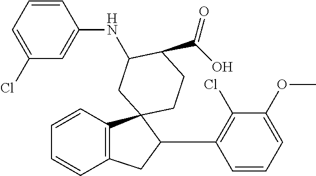

Using General procedure 19 and Example 267 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected and purified via prep RP-HPLC using 25 mM aq. NH₄HCO₃ solution and MeCN as eluents to obtain Example 291. HRMS calculated for C₂₈H₂₇NO₃Cl₂: 495.1368; found: 496.1451 (M+H).

The enantiomer eluting later was collected and purified via prep RP-HPLC using 25 mM aq. NH₄HCO₃ solution and MeCN as eluents to obtain Example 292. HRMS calculated for C₂₈H₂₇NO₃Cl₂: 495.1368; found: 496.1441 (M+H).

### Example 293 (1r,4r)-2′-(1-benzofuran-7-yl)-4-(3-chloroanilino)-2′,3′-dihydrospiro[cyclohexane-1,1′-indene]-4-carboxylic acid, enantiomer 1

and

### Example 294 (1r,4r)-2′-(1-benzofuran-7-yl)-4-(3-chloroanilino)-2′,3′-dihydrospiro[cyclohexane-1,1′-indene]-4-carboxylic acid, enantiomer 2

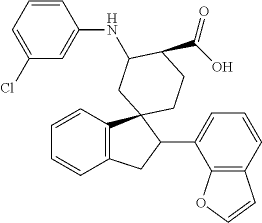

and

### Example 295 (1r,4r)-4-(3-chloroanilino)-2′-(2,3-dihydro-1-benzofuran-7-yl)-2′,3′-dihydrospiro[cyclohexane-1,1′-indene]-4-carboxylic acid, enantiomer 1

and

### Example 296 (1r,4r)-4-(3-chloroanilino)-2′-(2,3-dihydro-1-benzofuran-7-yl)-2′,3′-dihydrospiro[cyclohexane-1,1′-indene]-4-carboxylic acid, enantiomer 2

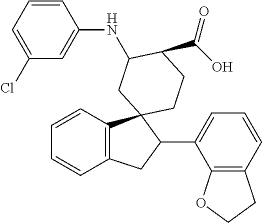

Using General procedure 19 and Example 266 as the appropriate indene and using AcOH instead of EtOAc, a partial saturation of the furan ring occurred. The mixture was separated via prep RP-HPLC using 25 mM aq. NH₄HCO₃ solution and MeCN as eluents. Both racemates were separated via chiral chromatography. Column: AD, 50×500 mm, 20 μm, eluents: EtOH+0.05% HCOOH. The enantiomer eluting earlier was collected and purified via prep RP-HPLC using 25 mM aq. NH₄HCO₃ solution and MeCN as eluents to obtain Example 293. HRMS calculated for C₂₉H₂₆NO₃Cl: 471.1601; found: 472.1675 (M+H).

The enantiomer eluting later was collected and purified via prep RP-HPLC using 25 mM aq. NH₄HCO₃ solution and MeCN as eluents to obtain Example 294. HRMS calculated for C₂₉H₂₆NO₃Cl: 471.1601; found: 472.1666 (M+H).

The enantiomer eluting earlier was collected and purified via prep RP-HPLC using 25 mM aq. NH₄HCO₃ solution and MeCN as eluents and obtain Example 295. HRMS calculated for C₂₉H₂₈NO₃Cl: 473.1758; found: 474.1842 (M+H).

The enantiomer eluting later was collected and purified via prep RP-HPLC using 25 mM aq. NH₄HCO₃ solution and MeCN as eluents to obtain Example 296. HRMS calculated for C₂₉H₂₈NO₃Cl: 473.1758; found: 474.1835 (M+H).

### Example 297 (1s,4s)-4-(3-chloroanilino)-2′-(4-methylphenyl)spiro[cyclohexane-1,1′-indene]-4-carboxylic acid

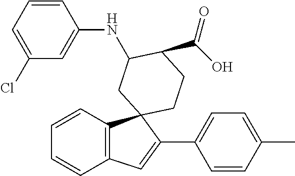 [SMILES: Cc1ccc(C2=Cc3ccccc3[C@@]23CC[C@H](C(=O)O)C(Nc2cccc(Cl)c2)C3)cc1]

Using General procedure 18 and Preparation 6a as the appropriate 2-bromo-indene and p-tolylboronic acid as the appropriate boronic acid, Example 297 was obtained. ¹H NMR (500 MHz, DMSO-d₆) δ ppm: 12.83 (br, 1H), 7.77 (d, 1H), 7.71 (d, 2H), 7.42 (d, 1H), 7.29 (t, 1H), 7.21 (s, 1H), 7.20 (d, 2H), 7.20 (t, 1H), 7.14 (t, 1H), 6.69 (t, 1H), 6.65 (dd, 1H), 6.60 (dd, 1H), 6.54 (br, 1H), 2.52/1.03 (t+d, 4H), 2.44/2.29 (td+d, 4H), 2.33 (s, 3H). HRMS calculated for C₂₈H₂₆NO₂Cl: 443.1652; found: 444.1720 (M+H).

### Example 298 (1s,4s)-4-(3-chloroanilino)-2′-(4-chlorophenyl)spiro[cyclohexane-1,1′-indene]-4-carboxylic acid

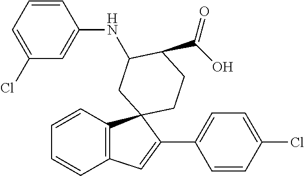 [SMILES: O=C(O)[C@H]1CC[C@@]2(CC1Nc1cccc(Cl)c1)C(c1ccc(Cl)cc1)=Cc1ccccc12]

Using General procedure 18 and Preparation 6a as the appropriate 2-bromo-indene and (4-chlorophenyl)boronic acid as the appropriate boronic acid, Example 298 was obtained. ¹H NMR (500 MHz, DMSO-d₆) δ ppm: 12.86 (br, 1H), 7.83 (d, 2H), 7.78 (d, 1H), 7.45 (d, 1H), 7.42 (d, 2H), 7.31 (s, 1H), 7.31 (t, 1H), 7.24 (t, 1H), 7.15 (t, 1H), 6.68 (t, 1H), 6.66 (dd, 1H), 6.61 (dd, 1H), 6.54 (br, 1H), 2.44/1.04 (m+d, 4H), 2.44/2.28 (m+d, 4H). HRMS calculated for C₂₇H₂₃NO₂Cl₂: 463.1106; found: 464.1182 (M+H).

### Example 299 (1s,4s)-4-(3-chloroanilino)-2′-(3-methoxy-2-methylphenyl)spiro[cyclohexane-1,1′-indene]-4-carboxylic acid

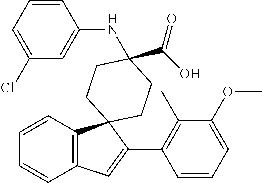 [SMILES: COc1cccc(C2=Cc3ccccc3[C@]23CC[C@@](Nc2cccc(Cl)c2)(C(=O)O)CC3)c1C]

Using General procedure 18 and Preparation 6a as the appropriate 2-bromo-indene and (3-methoxy-2-methyl-phenyl)boronic acid as the appropriate boronic acid, Example 299 was obtained. HRMS calculated for C₂₉H₂₈NO₃Cl: 473.1758; found: 474.1839 (M+H).

### Example 300 (1s,4s)-4-(3-chloroanilino)-2′-(3-phenoxyphenyl)spiro[cyclohexane-1,1′-indene]-4-carboxylic acid

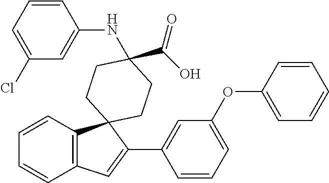 [SMILES: O=C(O)[C@]1(Nc2cccc(Cl)c2)CC[C@]2(CC1)C(c1cccc(Oc3ccccc3)c1)=Cc1ccccc12]

Using General procedure 18 and Preparation 6a as the appropriate 2-bromo-indene and (3-phenoxyphenyl)boronic acid as the appropriate boronic acid, Example 300 was obtained. HRMS calculated for C₃₃H₂₈NO₃Cl: 521.1758; found: 522.1826 (M+H).

### Example 301 (1s,4s)-2′-(3-benzylphenyl)-4-(3-chloroanilino)spiro[cyclohexane-1,1′-indene]-4-carboxylic acid

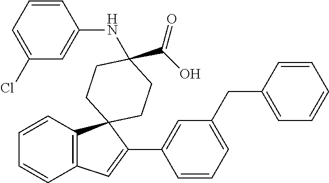 [SMILES: O=C(O)[C@]1(Nc2cccc(Cl)c2)CC[C@]2(CC1)C(c1cccc(Cc3ccccc3)c1)=Cc1ccccc12]

Using General procedure 18 and Preparation 6a as the appropriate 2-bromo-indene and (3-benzylphenyl)boronic acid as the appropriate boronic acid, Example 301 was obtained. HRMS calculated for C₃₄H30NO₂Cl: 519.1965; found: 520.2035 (M+H).

### Example 302 (1s,4s)-2′-[3-(benzyloxy)phenyl]-4-(3-chloroanilino)spiro[cyclohexane-1,1′-indene]-4-carboxylic acid

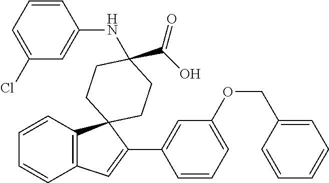 [SMILES: O=C(O)[C@]1(Nc2cccc(Cl)c2)CC[C@]2(CC1)C(c1cccc(OCc3ccccc3)c1)=Cc1ccccc12]

Using General procedure 18 and Preparation 6a as the appropriate 2-bromo-indene and (3-benzyloxyphenyl)boronic acid as the appropriate boronic acid, Example 302 was obtained. HRMS calculated for C₃₄H₃₀NO₃Cl: 535.1914; found: 536.1976 (M+H).

### Example 303 (1s,4s)-2′-(1-benzofuran-2-yl)-4-(3-chloroanilino)spiro[cyclohexane-1,1′-indene]-4-carboxylic acid

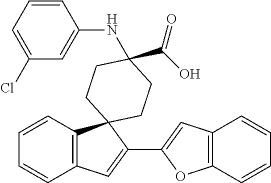 [SMILES: O=C(O)[C@]1(Nc2cccc(Cl)c2)CC[C@]2(CC1)C(c1cc3ccccc3o1)=Cc1ccccc12]

Using General procedure 18 and Preparation 6a as the appropriate 2-bromo-indene and benzofuran-2-ylboronic acid as the appropriate boronic acid, Example 303 was obtained. HRMS calculated for C₂₉H₂₄NO₃Cl: 469.1445; found: 470.1519 (M+H).

### Example 304 (1s,4s)-4-(3-chloroanilino)-2′-(3-hydroxyphenyl)spiro[cyclohexane-1,1′-indene]-4-carboxylic acid

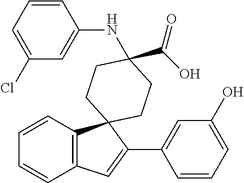 [SMILES: O=C(O)[C@]1(Nc2cccc(Cl)c2)CC[C@]2(CC1)C(c1cccc(O)c1)=Cc1ccccc12]

Using General procedure 18 and Preparation 6a as the appropriate 2-bromo-indene and (3-hydroxyphenyl)boronic acid as the appropriate boronic acid, Example 304 was obtained. HRMS calculated for C₂₇H₂₄NO₃Cl: 445.1445; found: 446.1517 (M+H).

### Example 305 (1s,4s)-4-(3-chloroanilino)-2′-phenylspiro[cyclohexane-1,1′-indene]-4-carboxylic acid

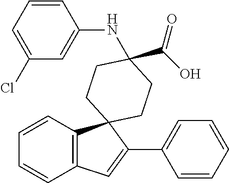 [SMILES: O=C(O)[C@]1(Nc2cccc(Cl)c2)CC[C@]2(CC1)C(c1ccccc1)=Cc1ccccc12]

Using General procedure 18 and Preparation 6a as the appropriate 2-bromo-indene and phenylboronic acid as the appropriate boronic acid, Example 305 was obtained. HRMS calculated for C₂₇H₂₄NO₂Cl: 429.1496; found: 430.1564 (M+H).

### Example 306 (1r,4r)-4-(3-chloroanilino)-2′-(4-methylphenyl)-2′,3′-dihydrospiro[cyclohexane-1,1′-indene]-4-carboxylic acid, enantiomer 1

and

### Example 307 (1r,4r)-4-(3-chloroanilino)-2′-(4-methylphenyl)-2′,3′-dihydrospiro[cyclohexane-1,1′-indene]-4-carboxylic acid, enantiomer 2

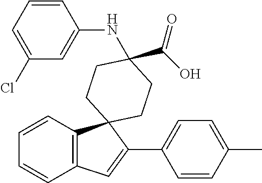 [SMILES: Cc1ccc(C2=Cc3ccccc3[C@]23CC[C@@](Nc2cccc(Cl)c2)(C(=O)O)CC3)cc1]

Using General procedure 19 and Example 297 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 306. ¹H NMR (500 MHz, DMSO-d₆) δ ppm: 12.66 (br s, 1H), 7.31-7.15 (m, 4H), 7.01 (m, 2H), 7.01 (t, 1H), 6.92 (m, 2H), 6.52 (dm, 1H), 6.51 (t, 1H), 6.44 (dm, 1H), 6.01 (br s, 1H), 3.38 (m, 1H), 3.35/2.95 (m+m, 2H), 2.43-1.23 (m, 8H), 2.23 (s, 3H). HRMS calculated for C₂₈H₂₈NO₂Cl: 445.1808; found: 446.1868 (M+H).

The enantiomer eluting later was collected as Example 307. HRMS calculated for C₂₈H₂₈NO₂Cl: 445.1808; found: 446.1880 (M+H).

### Example 308 (1s,4s)-4-(3-chloroanilino)-2′-(3,5-dimethoxyphenyl)spiro[cyclohexane-1,1′-indene]-4-carboxylic acid

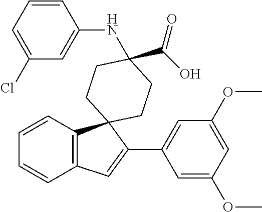 [SMILES: COc1cc(OC)cc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (3,5-dimethoxyphenyl)boronic acid as the appropriate boronic acid, Example 308 was obtained. HRMS calculated for C₂₉H₂₈NO₄Cl: 489.1707; found: 490.1784 (M+H).

### Example 309 (1s,4s)-2′-[4-(benzyloxy)phenyl]-4-(3-chloroanilino)spiro[cyclohexane-1,1′-indene]-4-carboxylic acid

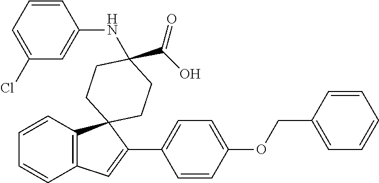 [SMILES: O=C(O)[C@]1(Nc2cccc(Cl)c2)CC[C@]2(CC1)C(c1ccc(OCc3ccccc3)cc1)=Cc1ccccc12]

Using General procedure 18 and Preparation 6a as the appropriate 2-bromo-indene and (4-benzyloxyphenyl)boronic acid as the appropriate boronic acid, Example 309 was obtained. HRMS calculated for C₃₄H₃₀NO₃Cl: 535.1914; found: 536.1998 (M+H).

### Example 310 (1s,4s)-4-(3-chloroanilino)-2′-(3-methoxy-5-methylphenyl)spiro[cyclohexane-1,1′-indene]-4-carboxylic acid

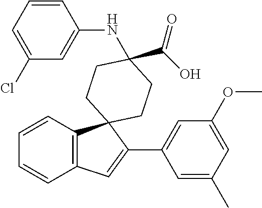 [SMILES: COc1cc(C)cc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (3-methoxy-5-methyl-phenyl)boronic acid as the appropriate boronic acid, Example 310 was obtained. HRMS calculated for C₂₉H₂₈NO₃Cl: 473.1758; found: 474.1835 (M+H).

### Example 311 (1s,4s)-4-(3-chloroanilino)-2′-(2-chlorophenyl)spiro[cyclohexane-1,1′-indene]-4-carboxylic acid

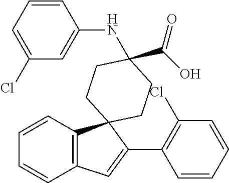 [SMILES: O=C(O)[C@]1(Nc2cccc(Cl)c2)CC[C@]2(CC1)C(c1ccccc1Cl)=Cc1ccccc12]

Using General procedure 18 and Preparation 6a as the appropriate 2-bromo-indene and (2-chlorophenyl)boronic acid as the appropriate boronic acid, Example 311 was obtained. HRMS calculated for C₂₇H₂₃NO₂Cl₂: 463.1106; found: 464.1172 (M+H).

### Example 312 (1s,4s)-4-(3-chloroanilino)-2′-[3-(trifluoromethyl)phenyl]spiro[cyclohexane-1,1′-indene]-4-carboxylic acid

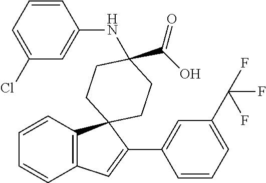 [SMILES: O=C(O)[C@]1(Nc2cccc(Cl)c2)CC[C@]2(CC1)C(c1cccc(C(F)(F)F)c1)=Cc1ccccc12]

Using General procedure 18 and Preparation 6a as the appropriate 2-bromo-indene and [3-(trifluoromethyl)phenyl]boronic acid as the appropriate boronic acid, Example 312 was obtained. HRMS calculated for C₂₈H23NO₂F₃Cl: 497.1369; found: 498.1435 (M+H).

### Example 313 (1s,4s)-4-(3-chloroanilino)-2′-(3-ethoxyphenyl)spiro[cyclohexane-1,1′-indene]-4-carboxylic acid

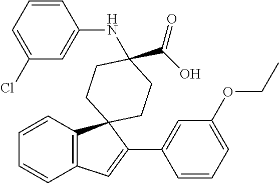 [SMILES: CCOc1cccc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (3-ethoxyphenyl)boronic acid as the appropriate boronic acid, Example 313 was obtained. HRMS calculated for C₂₉H₂₈NO₃Cl: 473.1758; found: 474.1821 (M+H).

### Example 314 (1s,4s)-4-(3-chloroanilino)-2′-[3-(trifluoromethoxy)phenyl]spiro[cyclohexane-1,1′-indene]-4-carboxylic acid

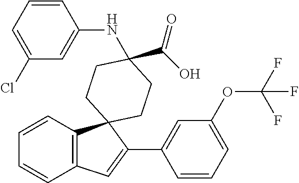 [SMILES: O=C(O)[C@]1(Nc2cccc(Cl)c2)CC[C@]2(CC1)C(c1cccc(OC(F)(F)F)c1)=Cc1ccccc12]

Using General procedure 18 and Preparation 6a as the appropriate 2-bromo-indene and [3-(trifluoromethoxy)phenyl]boronic acid as the appropriate boronic acid, Example 314 was obtained. HRMS calculated for C₂₈H23NO₃F₃Cl: 513.1318; found: 514.1340 (M+H).

### Example 315 (1s,4s)-4-(3-chloroanilino)-2′-[3-(2-methoxyethoxy)phenyl]spiro[cyclohexane-1,1′-indene]-4-carboxylic acid

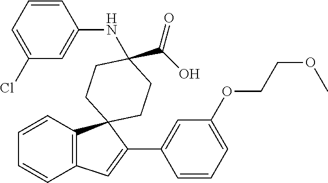 [SMILES: COCCOc1cccc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and [3-(2-methoxyethoxy)phenyl]boronic acid as the appropriate boronic acid, Example 315 was obtained. HRMS calculated for C₃₀H₃₀NO₄Cl: 503.1863; found: 504.1949 (M+H).

### Example 316 (1s,4s)-4-(3-chloroanilino)-2′-(4-methylthiophen-2-yl)spiro[cyclohexane-1,1′-indene]-4-carboxylic acid

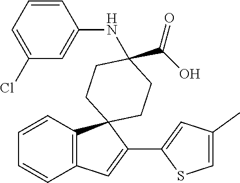 [SMILES: Cc1csc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and 4,4,5,5-tetramethyl-2-(4-methyl-2-thienyl)-1,3,2-dioxaborolane as the appropriate boronic ester, Example 316 was obtained. HRMS calculated for C₂₆H₂₄NO₂SCl: 449.1216; found: 450.1298 (M+H).

### Example 317 (1s,4s)-4-(3-chloroanilino)-2′-(5-phenylfuran-2-yl)spiro[cyclohexane-1,1′-indene]-4-carboxylic acid

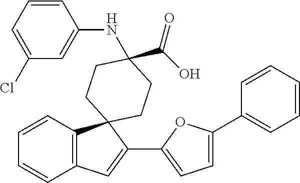 [SMILES: O=C(O)[C@]1(Nc2cccc(Cl)c2)CC[C@]2(CC1)C(c1ccc(-c3ccccc3)o1)=Cc1ccccc12]

Using General procedure 18 and Preparation 6a as the appropriate 2-bromo-indene and 4,4,5,5-tetramethyl-2-(5-phenyl-2-furyl)-1,3,2-dioxaborolane as the appropriate boronic ester, Example 317 was obtained. HRMS calculated for C₃₁H₂₆NO₃Cl: 495.1601; found: 496.1675 (M+H).

### Example 318 (1s,4s)-4-(3-chloroanilino)-2′-(5-methylthiophen-3-yl)spiro[cyclohexane-1,1′-indene]-4-carboxylic acid

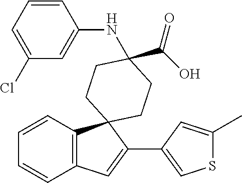 [SMILES: Cc1cc(C2=Cc3ccccc3[C@]23CC[C@@](Nc2cccc(Cl)c2)(C(=O)O)CC3)cs1]

Using General procedure 18 and Preparation 6a as the appropriate 2-bromo-indene and (5-methyl-3-thienyl)boronic acid as the appropriate boronic acid, Example 318 was obtained. HRMS calculated for C₂₆H₂₄NO₂SCl: 449.1216; found: 450.1297 (M+H).

### Example 319 (1r,4r)-4-(3-chloroanilino)-2′-(4-chlorophenyl)-2′,3′-dihydrospiro[cyclohexane-1,1′-indene]-4-carboxylic acid, enantiomer 1

and

### Example 320 (1r,4r)-4-(3-chloroanilino)-2′-(4-chlorophenyl)-2′,3′-dihydrospiro[cyclohexane-1,1′-indene]-4-carboxylic acid, enantiomer 2

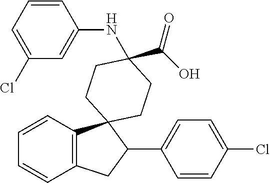

Using General procedure 19 and Example 298 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 319. HRMS calculated for C₂₇H₂₅NO₂Cl₂: 465.1262; found: 466.1330 (M+H).

The enantiomer eluting later was collected as Example 320. HRMS calculated for C₂₇H₂₅NO₂Cl₂: 465.1262; found: 466.1343 (M+H).

### Example 321 (1r,4r)-4-(3-chloroanilino)-2′-(3-methoxy-2-methylphenyl)-2′,3′-dihydrospiro[cyclohexane-1,1′-indene]-4-carboxylic acid, enantiomer 1

and

### Example 322 (1r,4r)-4-(3-chloroanilino)-2′-(3-methoxy-2-methylphenyl)-2′,3′-dihydrospiro[cyclohexane-1,1′-indene]-4-carboxylic acid, enantiomer 2

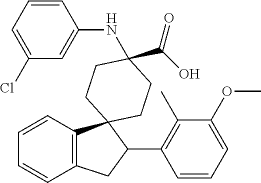

Using General procedure 19 and Example 299 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 321. HRMS calculated for C₂₉H₃₀NO₃Cl: 475.1914; found: 476.1994 (M+H).

The enantiomer eluting later was collected as Example 322. HRMS calculated for C₂₉H₃₀NO₃Cl: 475.1914; found: 476.1981 (M+H).

### Example 323 (1r,2′S,4S)-4-(3-chloroanilino)-2′-(3-phenoxyphenyl)-2′,3′-dihydrospiro[cyclohexane-1,1′-indene]-4-carboxylic acid

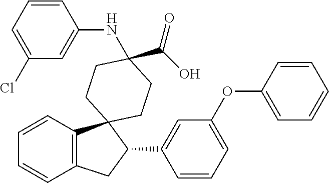 [SMILES: O=C(O)[C@]1(Nc2cccc(Cl)c2)CC[C@@]2(CC1)c1ccccc1C[C@H]2c1cccc(Oc2ccccc2)c1]

and

### Example 324 (1r,2′R,4R)-4-(3-chloroanilino)-2′-(3-phenoxyphenyl)-2′,3′-dihydrospiro[cyclohexane-1,1′-indene]-4-carboxylic acid

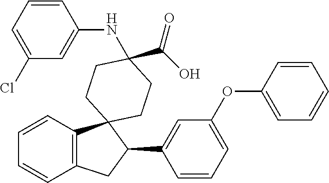 [SMILES: O=C(O)[C@]1(Nc2cccc(Cl)c2)CC[C@@]2(CC1)c1ccccc1C[C@@H]2c1cccc(Oc2ccccc2)c1]

Using General procedure 19 and Example 300 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 323. ¹H NMR (500 MHz, DMSO-d₆) δ ppm: 7.32-7.15 (m, 4H), 7.23 (m, 2H), 7.23 (t, 1H), 7.03 (tm, 1H), 7.03 (t, 1H), 6.88 (br, 3H), 6.87 (dm, 2H), 6.81 (dm, 1H), 6.74 (br, 1H), 6.54 (dm, 1H), 6.53 (t, 1H), 6.45 (dm, 1H), 6.04 (br s, 1H), 3.40 (dd, 1H), 3.32/3.02 (dd+dd, 2H), 2.42-1.27 (m, 8H). HRMS calculated for C₃₃H30NO₃Cl: 523.1914; found: 524.1990 (M+H).

The enantiomer eluting later was collected as Example 324. HRMS calculated for C₃₃H30NO₃Cl: 523.1914; found: 524.1988 (M+H).

### Example 325 (1r,2′S,4S)-2′-(3-benzylphenyl)-4-(3-chloroanilino)-2′,3′-dihydrospiro[cyclohexane-1,1′-indene]-4-carboxylic acid

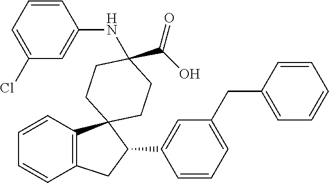 [SMILES: O=C(O)[C@]1(Nc2cccc(Cl)c2)CC[C@@]2(CC1)c1ccccc1C[C@H]2c1cccc(Cc2ccccc2)c1]

and

### Example 326 (1r,2′R,4R)-2′-(3-benzylphenyl)-4-(3-chloroanilino)-2′,3′-dihydrospiro[cyclohexane-1,1′-indene]-4-carboxylic acid

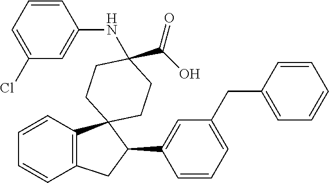 [SMILES: O=C(O)[C@]1(Nc2cccc(Cl)c2)CC[C@@]2(CC1)c1ccccc1C[C@@H]2c1cccc(Cc2ccccc2)c1]

Using General procedure 19 and Example 301 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 325. ¹H NMR (500 MHz, DMSO-d₆) δ ppm: 12.64 (br s, 1H), 7.30-7.17 (m, 4H), 7.17-6.82 (m, 9H), 7.02 (t, 1H), 6.54 (dm, 1H), 6.51 (t, 1H), 6.43 (dm, 1H), 5.98 (br s, 1H), 3.84 (s, 2H), 3.36 (m, 1H), 3.33/2.99 (m+dd, 2H), 2.41-1.17 (m, 8H). HRMS calculated for C₃₄H₃₂NO₂Cl: 521.2122; found: 522.2191 (M+H).

The enantiomer eluting later was collected as Example 326. HRMS calculated for C₃₄H₃₂NO₂Cl: 521.2122; found: 522.2185 (M+H).

### Example 327 (1r,4r)-4-(3-chloroanilino)-2′-phenyl-2′,3′-dihydrospiro[cyclohexane-1,1′-indene]-4-carboxylic acid, enantiomer 2

and

### Example 328 (1r,4r)-4-(3-chloroanilino)-2′-phenyl-2′,3′-dihydrospiro[cyclohexane-1,1′-indene]-4-carboxylic acid, enantiomer 1

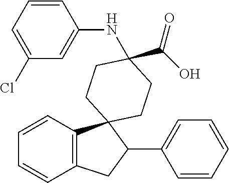

Using General procedure 19 and Example 305 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting later was collected as Example 327. HRMS calculated for C₂₇H₂₆NO₂Cl: 431.1652; found: 432.1730 (M+H).

The enantiomer eluting earlier was collected as Example 328. HRMS calculated for C₂₇H₂₆NO₂Cl: 431.1652; found: 432.1724 (M+H).

### Example 329 (1s,4s)-4-(3-chloroanilino)-2′-(furan-3-yl)spiro[cyclohexane-1,1′-indene]-4-carboxylic acid

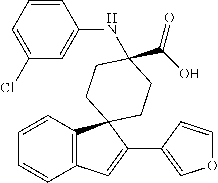 [SMILES: O=C(O)[C@]1(Nc2cccc(Cl)c2)CC[C@]2(CC1)C(c1ccoc1)=Cc1ccccc12]

Using General procedure 18 and Preparation 6a as the appropriate 2-bromo-indene and 2-(3-furyl)-4,4,5,5-tetramethyl-1,3,2-dioxaborolane as the appropriate boronic ester, Example 329 was obtained. HRMS calculated for C₂₅H₂₂NO₃Cl: 419.1288; found: 420.1351 (M+H).

### Example 330 (1s,4s)-4-(3-chloroanilino)-2′-(thiophen-3-yl)spiro[cyclohexane-1,1′-indene]-4-carboxylic acid

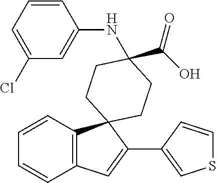 [SMILES: O=C(O)[C@]1(Nc2cccc(Cl)c2)CC[C@]2(CC1)C(c1ccsc1)=Cc1ccccc12]

Using General procedure 18 and Preparation 6a as the appropriate 2-bromo-indene and 3-thienylboronic acid as the appropriate boronic acid, Example 330 was obtained. HRMS calculated for C₂₅H₂₂NO₂SCl: 435.1060; found: 436.1135 (M+H).

### Example 331 (1s,4s)-4-(3-chloroanilino)-2′-(3-chloro-5-methoxyphenyl)spiro[cyclohexane-1,1′-indene]-4-carboxylic acid

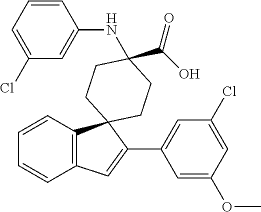 [SMILES: COc1cc(Cl)cc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and (3-chloro-5-methoxy-phenyl)boronic acid as the appropriate boronic acid, Example 331 was obtained. HRMS calculated for C₂₈H₂₅NO₃Cl₂: 493.1212; found: 494.1300 (M+H).

### Example 332 (1r,2′S,4S)-2′-[3-(benzyloxy)phenyl]-4-(3-chloroanilino)-2′,3′-dihydrospiro[cyclohexane-1,1′-indene]-4-carboxylic acid

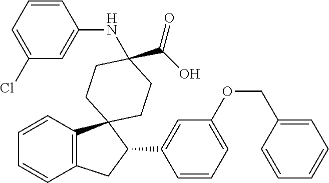 [SMILES: O=C(O)[C@]1(Nc2cccc(Cl)c2)CC[C@@]2(CC1)c1ccccc1C[C@H]2c1cccc(OCc2ccccc2)c1]

and

### Example 333 (1r,2′R,4R)-2′-[3-(benzyloxy)phenyl]-4-(3-chloroanilino)-2′,3′-dihydrospiro[cyclohexane-1,1′-indene]-4-carboxylic acid

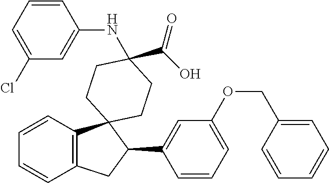 [SMILES: O=C(O)[C@]1(Nc2cccc(Cl)c2)CC[C@@]2(CC1)c1ccccc1C[C@@H]2c1cccc(OCc2ccccc2)c1]

Using General procedure 19 and Example 302 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 332. ¹H NMR (500 MHz, DMSO-d₆) δ ppm: 12.64 (br s, 1H), 7.38-7.16 (m, 9H), 7.11 (t, 1H), 7.01 (t, 1H), 6.83 (m, 1H), 6.65 (br s, 1H), 6.62 (dm, 1H), 6.52 (m, 1H), 6.52 (m, 1H), 6.45 (m, 1H), 6.01 (br s, 1H), 4.99/4.97 (d+d, 2H), 3.38 (m, 1H), 3.36/2.69 (m+m, 2H), 2.42-1.24 (m, 8H). HRMS calculated for C₃₄H₃₂NO₃Cl: 537.2071; found: 538.2134 (M+H).

The enantiomer eluting later was collected as Example 333. HRMS calculated for C₃₄H₃₂NO₃Cl: 537.2071; found: 538.2141 (M+H).

### Example 334 (1s,4s)-4-(3-chloroanilino)-2′-(3-chlorophenyl)spiro[cyclohexane-1,1′-indene]-4-carboxylic acid

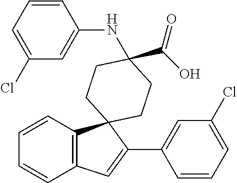 [SMILES: O=C(O)[C@]1(Nc2cccc(Cl)c2)CC[C@]2(CC1)C(c1cccc(Cl)c1)=Cc1ccccc12]

Using General procedure 18 and Preparation 6a as the appropriate 2-bromo-indene and (3-chlorophenyl)boronic acid as the appropriate boronic acid, Example 334 was obtained. HRMS calculated for C₂₇H₂₃NO₂Cl₂: 463.1106; found: 464.1164 (M+H).

### Example 335 (1s,4s)-4-(3-chloroanilino)-2′-(3-methylphenyl)spiro[cyclohexane-1,1′-indene]-4-carboxylic acid

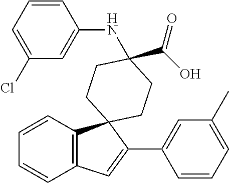 [SMILES: Cc1cccc(C2=Cc3ccccc3[C@]23CC[C@@](Nc2cccc(Cl)c2)(C(=O)O)CC3)c1]

Using General procedure 18 and Preparation 6a as the appropriate 2-bromo-indene and 4,4,5,5-tetramethyl-2-(m-tolyl)-1,3,2-dioxaborolane as the appropriate boronic ester, Example 335 was obtained. HRMS calculated for C₂₈H₂₆NO₂Cl: 443.1652; found: 444.1735 (M+H).

### Example 336 (1r,2′S,4S)-4-(3-chloroanilino)-2′-(3,5-dimethoxyphenyl)-2′,3′-dihydrospiro[cyclohexane-1,1′-indene]-4-carboxylic acid

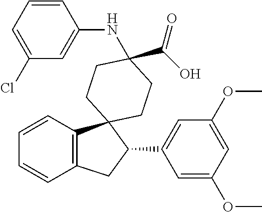 [SMILES: COc1cc(OC)cc([C@@H]2Cc3ccccc3[C@]23CC[C@@](Nc2cccc(Cl)c2)(C(=O)O)CC3)c1]

Using General procedure 19 and Example 308 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 336. HRMS calculated for C₂₉H₃₀NO₄Cl: 491.1863; found: 492.1947 (M+H).

### Example 337 (1r,2′R,4R)-2′-(1-benzofuran-2-yl)-4-(3-chloroanilino)-2′,3′-dihydrospiro[cyclohexane-1,1′-indene]-4-carboxylic acid

and

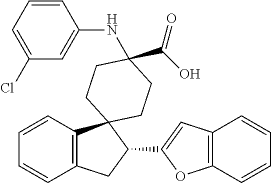 [SMILES: O=C(O)[C@]1(Nc2cccc(Cl)c2)CC[C@@]2(CC1)c1ccccc1C[C@H]2c1cc2ccccc2o1]

### Example 338 (1r,2′S,4S)-2′-(1-benzofuran-2-yl)-4-(3-chloroanilino)-2′,3′-dihydrospiro[cyclohexane-1,1′-indene]-4-carboxylic acid

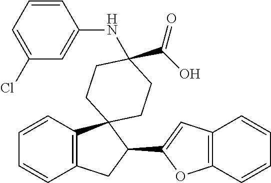 [SMILES: O=C(O)[C@]1(Nc2cccc(Cl)c2)CC[C@@]2(CC1)c1ccccc1C[C@@H]2c1cc2ccccc2o1]

Using General procedure 19 and Example 303 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 337. HRMS calculated for C₂₉H₂₆NO₃Cl: 471.1601; found: 472.1681 (M+H).

The enantiomer eluting later was collected as Example 338. HRMS calculated for C₂₉H₂₆NO₃Cl: 471.1601; found: 472.1689 (M+H).

### Example 339

### Example 339A cyclopropylmethyl (1s,4s)-4-(3-chloroanilino)-2′-[3-(cyclopropylmethoxy)phenyl]spiro[cyclohexane-1,1′-indene]-4-carboxylate

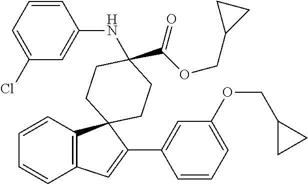 [SMILES: O=C(OCC1CC1)[C@]1(Nc2cccc(Cl)c2)CC[C@]2(CC1)C(c1cccc(OCC3CC3)c1)=Cc1ccccc12]

Example 304 (0.452 mg, 1 mmol) was dissolved in dry acetone (10 mL). Bromomethylcyclopropane (290 μL, 3 mmol) and K₂CO₃ (0.522 g, 4 mmol) were added and the mixture was stirred at 50° C. Several portions of the initial amount of bromomethylcyclopropane and K₂CO₃ were added during the reaction to reach acceptable conversion. The mixture was allowed to cool to rt, water and brine were added. The pH was set to 6-7 with 2 M aq. HCl solution. The mixture was extracted with DCM. The combined organic layers were dried over Na₂SO₄, filtered and the filtrate was concentrated under reduced pressure. The crude intermediate was purified via flash chromatography using heptane and EtOAc as eluents to obtain Example 339A. ¹H NMR (500 MHz, DMSO-d₆) δ ppm: 7.85-6.58 (m, 13H), 6.60 (s, 1H), 3.99 (d, 2H), 3.85 (d, 2H), 2.56/1.06 (t+d, 4H), 2.47/2.30 (td+d, 4H), 1.24 (m, 1H), 1.07 (m, 1H), 0.58/0.33 (m+m, 4H), 0.47/0.23 (m+m, 4H). HRMS calculated for C₃₅H₃₆ClNO₃: 553.2383; found 554.2462 (M+H).

### Example 339 (1s,4s)-4-(3-chloroanilino)-2′-[3-(cyclopropylmethoxy)phenyl]spiro[cyclohexane-1,1′-indene]-4-carboxylic acid

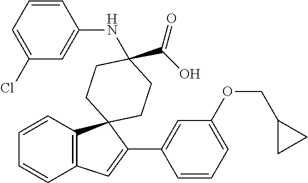 [SMILES: O=C(O)[C@]1(Nc2cccc(Cl)c2)CC[C@]2(CC1)C(c1cccc(OCC3CC3)c1)=Cc1ccccc12]

Using General procedure 33a and Example 339A as the appropriate ester, Example 339 was obtained. ¹H NMR (500 MHz, DMSO-d₆) δ ppm: 12.82 (br s, 1H), 7.82-7.17 (m, 4H), 7.35 (dm, 1H), 7.28 (m, 1H), 7.27 (t, 1H), 7.25 (s, 1H), 7.10 (t, 1H), 6.89 (dm, 1H), 6.66 (m, 1H), 6.65 (m, 1H), 6.59 (m, 1H), 6.51 (br s, 1H), 3.85 (d, 2H), 2.54/1.04 (m+m, 4H), 2.44/2.27 (m+m, 4H), 1.24 (m, 1H), 0.85/0.33 (m+m, 4H). HRMS calculated for C₃1H₃₀NO₃Cl: 499.1914; found: 500.1992 (M+H).

### Example 340 (1s,4s)-2′-(1-benzofuran-4-yl)-4-(3-chloroanilino)spiro[cyclohexane-1,1′-indene]-4-carboxylic acid

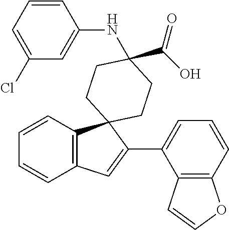 [SMILES: O=C(O)[C@]1(Nc2cccc(Cl)c2)CC[C@]2(CC1)C(c1cccc3occc13)=Cc1ccccc12]

Using General procedure 18 and Preparation 6a as the appropriate 2-bromo-indene and 2-(benzofuran-3-yl)-4,4,5,5-tetramethyl-1,3,2-dioxaborolane as the appropriate boronic ester, Example 340 was obtained. HRMS calculated for C₂₉H₂₄NO₃Cl: 469.1445; found: 470.1510 (M+H).

### Example 341 (1s,4s)-4-(3-chloroanilino)-2′-(4-phenoxyphenyl)spiro[cyclohexane-1,1′-indene]-4-carboxylic acid

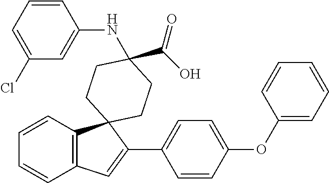 [SMILES: O=C(O)[C@]1(Nc2cccc(Cl)c2)CC[C@]2(CC1)C(c1ccc(Oc3ccccc3)cc1)=Cc1ccccc12]

Using General procedure 18 and Preparation 6a as the appropriate 2-bromo-indene and (4-phenoxyphenyl)boronic acid as the appropriate boronic acid, Example 341 was obtained. ¹H NMR (500 MHz, DMSO-d₆) δ ppm: 12.48 (br s, 1H), 7.84 (m, 2H), 7.82-7.03 (m, 9H), 7.23 (s, 1H), 7.07 (t, 1H), 7.00 (m, 2H), 6.66 (t, 1H), 6.59 (dm, 1H), 6.56 (dm, 1H), 6.50 (br s, 1H), 2.48/1.03 (m+m, 4H), 2.44/2.28 (m+m, 4H). HRMS calculated for C₃₃H₂₈NO₃Cl: 521.1758; found: 522.1827 (M+H).

### Example 342 (1s,4s)-4-(3-chloroanilino)-2′-{4-[(pyridin-4-yl)oxy]phenyl}spiro[cyclohexane-1,1′-indene]-4-carboxylic acid

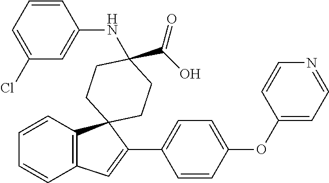 [SMILES: O=C(O)[C@]1(Nc2cccc(Cl)c2)CC[C@]2(CC1)C(c1ccc(Oc3ccncc3)cc1)=Cc1ccccc12]

Preparation 12a (460 mg, 1 mmol) was dissolved in DMF (3 mL). 4-Fluoropyridine hydrochloride (400 mg, 3 mmol) and Cs₂CO₃ (814 mg, 2.5 mmol) were added. The mixture was stirred at 80° C. until no further conversion was observed. The mixture was allowed to cool to rt, it was diluted with water. The precipitation was filtered, washed with water and dried under reduced pressure. Then it was hydrolyzed according to General procedure 33a to obtain Example 342. ¹H NMR (500 MHz, DMSO-d₆) δ ppm: 12.86 (br s, 1H), 8.50 (m, 2H), 7.91 (m, 2H), 7.80 (dm, 1H), 7.48-7.20 (m, 3H), 7.29 (s, 1H), 7.18 (m, 2H), 7.09 (t, 1H), 6.99 (m, 2H), 6.66 (t, 1H), 6.63 (dm, 1H), 6.57 (dm, 1H), 6.53 (br s, 1H), 2.48/1.06 (m+m, 4H), 2.45/2.29 (m+m, 4H). HRMS calculated for C₃₂H₂₇N₂O₃Cl: 522.1710; found: 523.1770 (M+H).

### Example 343 (1r,4r)-4-(3-chloroanilino)-2′-(3-chlorophenyl)-2′,3′-dihydrospiro[cyclohexane-1,1′-indene]-4-carboxylic acid, enantiomer 1

and

### Example 344 (1r,4r)-4-(3-chloroanilino)-2′-(3-chlorophenyl)-2′,3′-dihydrospiro[cyclohexane-1,1′-indene]-4-carboxylic acid, enantiomer 2

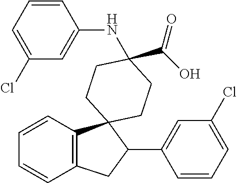

Using General procedure 19 and Example 334 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 343. ¹H NMR (500 MHz, DMSO-d₆) δ ppm: 12.64 (br s, 1H), 7.34-6.96 (m, 8H), 7.01 (t, 1H), 6.52 (dm, 1H), 6.51 (t, 1H), 6.44 (dm, 1H), 6.06 (br s, 1H), 3.44 (dd, 1H), 3.36/2.99 (dd+dd, 2H), 2.42-1.24 (m, 8H). HRMS calculated for C₂₇H₂₅NO₂C₂: 465.1262; found: 466.1336 (M+H).

The enantiomer eluting later was collected as Example 344. HRMS calculated for C₂₇H₂₅NO₂Cl₂: 465.1262; found: 466.1324 (M+H).

### Example 345 (1r,4r)-4-(3-chloroanilino)-2′-(3-methylphenyl)-2′,3′-dihydrospiro[cyclohexane-1,1′-indene]-4-carboxylic acid, enantiomer 1

and

### Example 346 (1r,4r)-4-(3-chloroanilino)-2′-(3-methylphenyl)-2′,3′-dihydrospiro[cyclohexane-1,1′-indene]-4-carboxylic acid, enantiomer 2

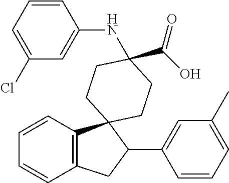

Using General procedure 19 and Example 335 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 345. ¹H NMR (500 MHz, DMSO-d₆) δ ppm: 12.63 (br s, 1H), 7.33-7.16 (m, 4H), 7.12-6.77 (m, 4H), 7.01 (t, 1H), 6.52 (dm, 1H), 6.50 (t, 1H), 6.43 (dm, 1H), 6.01 (br s, 1H), 3.35 (m, 1H), 3.34/2.98 (m+dd, 2H), 2.43-1.25 (m, 8H), 2.21 (s, 3H). HRMS calculated for C₂₈H₂₈NO₂Cl: 445.1808; found: 446.1884 (M+H).

The enantiomer eluting later was collected as Example 346. HRMS calculated for C₂₈H₂₈NO₂Cl: 445.1808; found: 446.1890 (M+H).

### Example 347 (1r,4r)-4-(3-chloroanilino)-2′-(3-chloro-5-methoxyphenyl)-2′,3′-dihydrospiro[cyclohexane-1,1′-indene]-4-carboxylic acid, enantiomer 1

and

### Example 348 (1r,4r)-4-(3-chloroanilino)-2′-(3-chloro-5-methoxyphenyl)-2′,3′-dihydrospiro[cyclohexane-1,1′-indene]-4-carboxylic acid, enantiomer 2

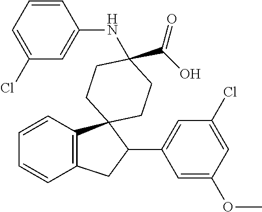

Using General procedure 19 and Example 331 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 347. ¹H NMR (500 MHz, DMSO-d₆) δ ppm: 12.67 (br s, 1H), 7.32-7.18 (m, 4H), 7.02 (t, 1H), 6.85/6.65/6.56 (t+br s+br s, 3H), 6.54 (t, 1H), 6.53 (dm, 1H), 6.46 (dm, 1H), 6.07 (br s, 1H), 3.68 (s, 3H), 3.41 (dd, 1H), 3.35/2.96 (dd+dd, 2H), 2.41-1.29 (m, 8H). HRMS calculated for C₂₈H₂₇NO₃Cl₂: 495.1368; found: 496.14312 (M+H).

The enantiomer eluting later was collected as Example 348. HRMS calculated for C₂₈H₂₇NO₃Cl₂: 495.1368; found: 496.1436 (M+H).

### Example 349 (1s,4s)-4-(3-chloroanilino)-2′-{4-[(pyridin-4-yl)methoxy]phenyl}spiro[cyclohexane-1,1′-indene]-4-carboxylic acid

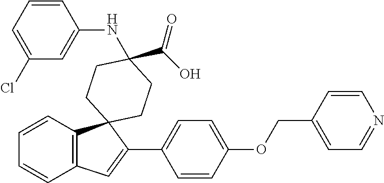 [SMILES: O=C(O)[C@]1(Nc2cccc(Cl)c2)CC[C@]2(CC1)C(c1ccc(OCc3ccncc3)cc1)=Cc1ccccc12]

Using General procedure 32 and Preparation 12a as the appropriate indene and 4-pyridylmethanol as the appropriate alcohol, Example 349 was obtained. ¹H NMR (500 MHz, DMSO-d₆) δ ppm: 12.70 (br s, 1H), 8.59 (m, 2H), 7.81-7.14 (m, 4H), 7.79 (m, 2H), 7.46 (m, 2H), 7.17 (s, 1H), 7.12 (t, 1H), 7.01 (m, 2H), 6.68 (t, 1H), 6.64 (dm, 1H), 6.59 (dm, 1H), 6.53 (br s, 1H), 5.25 (s, 2H), 2.49/1.00 (m+m, 4H), 2.44/2.27 (m+m, 4H). HRMS calculated for C₃₃H₂₉N₂O₃Cl: 536.1866; found: 537.1934 (M+H).

### Example 350 (1s,4s)-4-(3-chloroanilino)-2′-{4-[(pyridin-3-yl)methoxy]phenyl}spiro[cyclohexane-1,1′-indene]-4-carboxylic acid

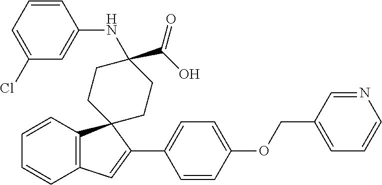 [SMILES: O=C(O)[C@]1(Nc2cccc(Cl)c2)CC[C@]2(CC1)C(c1ccc(OCc3cccnc3)cc1)=Cc1ccccc12]

Using General procedure 32 and Preparation 12a as the appropriate indene and 3-pyridylmethanol as the appropriate alcohol, Example 350 was obtained. ¹H NMR (500 MHz, DMSO-d₆) δ ppm: 12.86 (br s, 1H), 8.70 (d, 1H), 8.56 (dm, 1H), 7.90 (dm, 1H), 7.81-7.15 (m, 4H), 7.79 (m, 2H), 7.45 (m, 1H), 7.17 (s, 1H), 7.14 (t, 1H), 7.04 (m, 2H), 6.68 (t, 1H), 6.65 (dm, 1H), 6.60 (dm, 1H), 6.55 (s, 1H), 5.21 (s, 2H), 2.48/1.01 (m+m, 4H), 2.44/2.28 (m+m, 4H). HRMS calculated for C₃₃H₂₉N₂O₃Cl: 536.1866; found: 537.1934 (M+H).

### Example 351 (1s,4s)-4-(3-chloroanilino)-2′-{4-[(pyridin-3-yl)oxy]phenyl}spiro[cyclohexane-1,1′-indene]-4-carboxylic acid

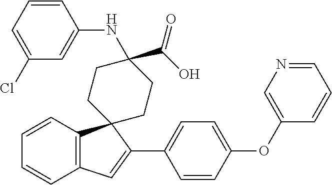 [SMILES: O=C(O)[C@]1(Nc2cccc(Cl)c2)CC[C@]2(CC1)C(c1ccc(Oc3cccnc3)cc1)=Cc1ccccc12]

Preparation 12a (230 mg, 0.5 mmol) was dissolved in DMF (1 mL). 3-Iodopyridine (103 mg, 0.5 mmol), 2,2,6,6-tetramethylheptane-3,5-dione (46 mg, 0.25 mmol), CuI (5 mg, 0.025 mmol) and Cs₂CO₃ (244 mg, 0.75 mmol) were added. The mixture was stirred at 60° C. until no further conversion was observed. The mixture was allowed to cool to rt, it was injected directly to the column and purified via flash chromatography using heptane and EtOAs as eluents. Then it was hydrolyzed according to General procedure 33a to obtain Example 351. ¹H NMR (500 MHz, DMSO-d₆) δ ppm: 12.39 (br s, 1H), 8.45 (d, 1H), 8.42 (dd, 1H), 7.87 (m, 2H), 7.82-7.17 (m, 4H), 7.51 (dm, 1H), 7.46 (m, 1H), 7.25 (s, 1H), 7.07 (t, 1H), 7.05 (m, 2H), 6.66 (t, 1H), 6.59 (dm, 1H), 6.55 (dm, 1H), 6.50 (br s, 1H), 2.46/1.02 (m+m, 4H), 2.43/2.28 (m+m, 4H). HRMS calculated for C₃₂H₂₇N₂O₃Cl: 522.1710; found: 523.1782 (M+H).

### Example 352 (1r,4r)-2′-[4-(benzyloxy)phenyl]-4-(3-chloroanilino)-2′,3′-dihydrospiro[cyclohexane-1,1′-indene]-4-carboxylic acid, enantiomer 1

and

### Example 353 (1r,4r)-2′-[4-(benzyloxy)phenyl]-4-(3-chloroanilino)-2′,3′-dihydrospiro[cyclohexane-1,1′-indene]-4-carboxylic acid, enantiomer 2

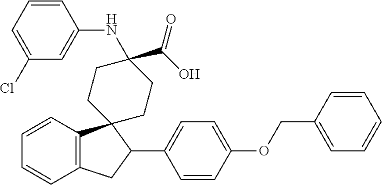

Using General procedure 19 and Example 309 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 352. ¹H NMR (500 MHz, DMSO-d₆) δ ppm: 12.58 (br, 1H), 7.45-7.29 (m, 5H), 7.30-7.17 (m, 4H), 7.01 (t, 1H), 6.96 (d, 2H), 6.85 (d, 2H), 6.52 (t, 1H), 6.52 (dm, 1H), 6.45 (dm, 1H), 6.03 (br, 1H), 5.02 (s, 2H), 3.36 (m, 1H), 3.35/2.94 (m+m, 2H), 2.43-1.25 (m, 8H). HRMS calculated for C₃₄H₃₂NO₃Cl: 537.2071; found: 538.2131 (M+H).

The enantiomer eluting later was collected as Example 353. HRMS calculated for C₃₄H₃₂NO₃Cl: 537.2071; found: 538.2144 (M+H).

### Example 354 (1r,4r)-4-(3-chloroanilino)-2′-[3-(cyclopropylmethoxy)phenyl]-2′,3′-dihydrospiro[cyclohexane-1,1′-indene]-4-carboxylic acid, enantiomer 1

and

### Example 355 (1r,4r)-4-(3-chloroanilino)-2′-[3-(cyclopropylmethoxy)phenyl]-2′,3′-dihydrospiro[cyclohexane-1,1′-indene]-4-carboxylic acid, enantiomer 2

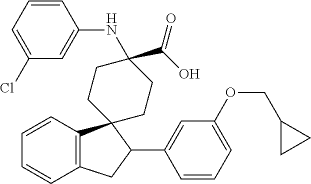

Using General procedure 19 and Example 339 as the appropriate indene and using AcOH instead of EtOAc, a racemate was obtained. The enantiomers were separated by chiral chromatography. Column: AD, 50×500 mm, 20 μm, eluents: EtOH/heptane+0.05% HCOOH. The enantiomer eluting earlier was collected as Example 354. HRMS calculated for C₃₁H₃₂NO₃Cl: 501.2071; found: 502.2143 (M+H).

The enantiomer eluting later was collected as Example 355. ¹H NMR (500 MHz, DMSO-d₆) δ ppm: 12.62 (br, 1H), 7.33-7.15 (m, 4H), 7.09 (t, 1H), 7.01 (t, 1H), 6.72 (dd, 1H), 6.59 (br d, 1H), 6.57 (br s, 1H), 6.53 (t, 1H), 6.52 (dm, 1H), 6.46 (dm, 1H), 6.04 (br, 1H), 3.70/3.68 (d+d, 2H), 3.36/2.96 (m+m, 2H), 3.36 (m, 1H), 2.42-1.29 (m, 8H), 1.13 (m, 1H), 0.51/0.25 (m+m, 4H). HRMS calculated for C₃₁H₃₂NO₃Cl: 501.2071; found: 502.2152 (M+H).

### Example 356 (1r,2′R,4R)-4-(3-chloroanilino)-2′-(5-phenylfuran-2-yl)-2′,3′-dihydrospiro[cyclohexane-1,1′-indene]-4-carboxylic acid

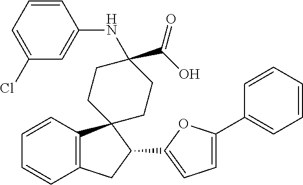 [SMILES: O=C(O)[C@]1(Nc2cccc(Cl)c2)CC[C@@]2(CC1)c1ccccc1C[C@H]2c1ccc(-c2ccccc2)o1]

and

### Example 357 (1r,2′S,4S)-4-(3-chloroanilino)-2′-(5-phenylfuran-2-yl)-2′,3′-dihydrospiro[cyclohexane-1,1′-indene]-4-carboxylic acid

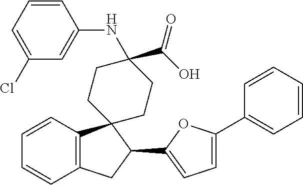 [SMILES: O=C(O)[C@]1(Nc2cccc(Cl)c2)CC[C@@]2(CC1)c1ccccc1C[C@@H]2c1ccc(-c2ccccc2)o1]

Using General procedure 19 and Example 317 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 356. HRMS calculated for C₃₁H₂₈NO₃CL: 497.1758; found: 498.1832 (M+H).

The enantiomer eluting later was collected as Example 357. HRMS calculated for C₃₁H₂₈NO₃Cl: 497.1758; found: 498.1843 (M+H).

### Example 358 (1r,2′S,4S)-4-(3-chloroanilino)-2′-(4-phenoxyphenyl)-2′,3′-dihydrospiro[cyclohexane-1,1′-indene]-4-carboxylic acid

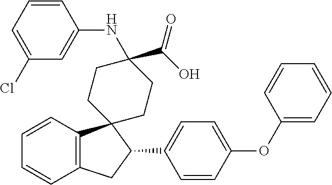 [SMILES: O=C(O)[C@]1(Nc2cccc(Cl)c2)CC[C@@]2(CC1)c1ccccc1C[C@H]2c1ccc(Oc2ccccc2)cc1]

and

### Example 359 (1r,2′R,4R)-4-(3-chloroanilino)-2′-(4-phenoxyphenyl)-2′,3′-dihydrospiro[cyclohexane-1,1′-indene]-4-carboxylic acid

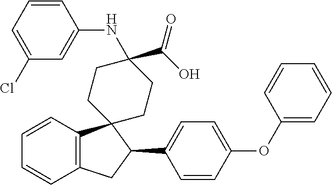 [SMILES: O=C(O)[C@]1(Nc2cccc(Cl)c2)CC[C@@]2(CC1)c1ccccc1C[C@@H]2c1ccc(Oc2ccccc2)cc1]

Using General procedure 19 and Example 341 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 358. ¹H NMR (500 MHz, DMSO-d₆) δ ppm: 12.73 (br, 1H), 7.36-7.17 (m, 4H), 7.36 (dm, 2H), 7.15-7.07 (m, 3H), 6.99 (t, 1H), 6.94 (dm, 2H), 6.89 (d, 2H), 6.51 (br s, 1H), 6.50 (d, 1H), 6.44 (d, 1H), 6.03 (br, 1H), 3.39 (t, 1H), 3.31/3.06 (dd+dd, 2H), 2.44-1.26 (m, 8H). HRMS calculated for C₃₃H30NO₃Cl: 523.1914; found: 524.1979 (M+H).

The enantiomer eluting later was collected as Example 359. HRMS calculated for C₃₃H30NO₃Cl: 523.1914; found: 524.1984 (M+H).

### Example 360

### Example 360A tert-butyl 4-[(1r,4r)-4-(3-chloroanilino)-4-(methoxycarbonyl)spiro[cyclohexane-1,1′-inden]-2′-yl]-3,6-dihydropyridine-1 (2H)-carboxylate

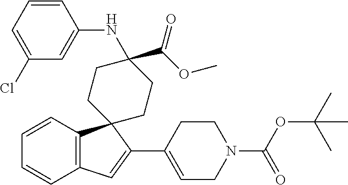 [SMILES: COC(=O)[C@]1(Nc2cccc(Cl)c2)CC[C@]2(CC1)C(C1=CCN(C(=O)OC(C)(C)C)CC1)=Cc1ccccc12]

Using General procedure 18 and Preparation 6c as the appropriate 2-bromo-indene and tert-butyl 4-(4,4,5,5-tetramethyl-1,3,2-dioxaborolan-2-yl)-3,6-dihydro-2H-pyridine-1-carboxylate as the appropriate boronic ester, Example 360A was obtained. ¹H NMR (500 MHz, DMSO-d₆) δ ppm: 7.74 (d, 1H), 7.40 (d, 1H), 7.27 (t, 1H), 7.19 (td, 1H), 7.12 (t, 1H), 6.81 (s, 1H), 6.68 (br, 1H), 6.63 (dd, 1H), 6.60 (s, 1H), 6.49 (br, 1H), 6.28 (m, 1H), 4.08 (m, 2H), 3.72 (s, 3H), 3.50 (t, 2H), 2.50-2.15 (m, 8H), 0.94 (br d, 2H).

### Example 360B methyl (1r,4r)-4-(3-chloroanilino)-2′-(1,2,3,6-tetrahydropyridin-4-yl)spiro[cyclohexane-1,1′-indene]-4-carboxylate

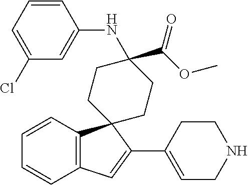 [SMILES: COC(=O)[C@]1(Nc2cccc(Cl)c2)CC[C@]2(CC1)C(C1=CCNCC1)=Cc1ccccc12]

Example 360A (1.053 g, 1.91 mmol) was dissolved in DCM (20 mL). TFA (2 mL) was added and the mixture was stirred at rt until no further conversion was observed. The mixture was concentrated under reduced pressure, then MeOH (NH₃, 1.2%, 40 mL) was added and the mixture was concentrated again under reduced pressure. The crude product was purified via flash chromatography using DCM and MeOH (1.2%) as eluents to obtain Example 360B. ¹H NMR (500 MHz, DMSO-d₆) δ ppm: 7.74 (d, 1H), 7.40 (d, 1H), 7.27 (t, 1H), 7.19 (td, 1H), 7.12 (t, 1H), 6.81 (s, 1H), 6.64 (br, 1H), 6.63 (dd, 1H), 6.60 (s, 1H), 6.50 (d, 1H), 6.32 (br, 1H), 3.71 (s, 3H), 3.58 (br, 2H), 3.04 (t, 2H), 2.50-2.22 (br, 8H), 0.94 (br d, 2H).

### Example 360C methyl (1r,4r)-2′-(1-benzyl-1,2,3,6-tetrahydropyridin-4-yl)-4-(3-chloroanilino)spiro[cyclohexane-1,1′-indene]-4-carboxylate

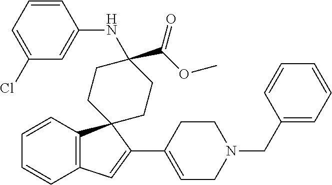 [SMILES: COC(=O)[C@]1(Nc2cccc(Cl)c2)CC[C@]2(CC1)C(C1=CCN(Cc3ccccc3)CC1)=Cc1ccccc12]

Example 360B (0.400 g, 0.89 mmol) was dissolved in DCE (6 mL). Benzaldehyde (0.117 mL, 1.15 mmol), Na(OAc)₃H (0.377 g, 1.78 mmol) and AcOH (0.51 mL) were added and the mixture was stirred at rt until no further conversion was observed. The mixture was concentrated under reduced pressure, then MeOH was added and the mixture was concentrated again under reduced pressure to obtain Example 360C. The crude product was used in the next step without further purification. LRMS calculated for C₃₄H₃₅N₂O₂Cl: 538.2; found: 539.2 (M+H).

### Example 360 (1r,4r)-2′-(1-benzyl-1,2,3,6-tetrahydropyridin-4-yl)-4-(3-chloroanilino)spiro[cyclohexane-1,1′-indene]-4-carboxylic acid

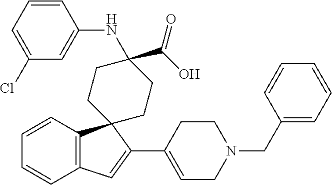 [SMILES: O=C(O)[C@]1(Nc2cccc(Cl)c2)CC[C@]2(CC1)C(C1=CCN(Cc3ccccc3)CC1)=Cc1ccccc12]

Using General procedure 33a and Example 360C as the appropriate ester, Example 360 was obtained. ¹H NMR (500 MHz, DMSO-d₆) δ ppm: 12.84 (br, 1H), 7.73 (d, 1H), 7.37 (d, 1H), 7.34 (m, 4H), 7.27 (m, 1H), 7.25 (t, 1H), 7.16 (t, 1H), 7.05 (t, 1H), 6.73 (s, 1H), 6.58 (t, 1H), 6.56 (dm, 1H), 6.51 (dm, 1H), 6.44 (br, 1H), 6.30 (br, 1H), 3.57 (s, 2H), 3.13 (br, 2H), 2.63 (t, 2H), 2.47-0.83 (m, 8H), 2.42 (br, 2H). HRMS calculated for C₃₃H₃₃N₂O₂Cl: 524.2231; found: 525.2302 (M+H).

### Example 361

### Example 361A 4-(benzofuran-4-yl)pyridine

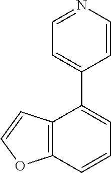 [SMILES: c1cc(-c2ccncc2)c2ccoc2c1]

A microwave vial was charged with 4-bromobenzofuran (1.43 g, 7.26 mmol), 4-pyridylboronic acid (1.071 g, 8.71 mmol), Cs₂CO₃ (3.548 g, 10.9 mmol), dioxane (15 mL) and water (7 mL). The vial was purged with N₂, followed by addition of Ataphos (0.257 g, 0.363 mmol). The mixture was heated at 110° C. for 15 min under microwave irradiation. The mixture was diluted with brine and extracted with DCM. The combined organic layers were dried over Na₂SO₄, filtered and the filtrate was concentrated under reduced pressure. The crude intermediate was purified via flash chromatography using heptane and EtOAc as eluents to obtain Example 361A. ¹H NMR (400 MHz, DMSO-d₆) δ ppm: 8.72-8.69 (m, 2H), 8.15 (d, 1H), 7.73 (d, 1H), 7.70-7.66 (m, 2H), 7.53-7.46 (m, 2H), 7.17 (m, 1H).

### Example 361B [4-(4-pyridyl)benzofuran-2-yl]boronic acid

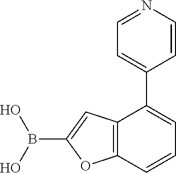 [SMILES: OB(O)c1cc2c(-c3ccncc3)cccc2o1]

A solution of Example 361A (0.195 g, 1 mmol) in dry THE (5 mL) was cooled to −78° C. under N₂ atmosphere and then nBuLi in hexanes (2.5 M, 0.42 mL, 1.05 mmol) was added dropwise.

After 30 minutes 2-isopropoxy-4,4,5,5-tetramethyl-1,3,2-dioxaborolane (0.224 mL, 1.1 mmol) was added dropwise. After 10 min the cooling bath was removed and the mixture was slowly allowed to warm up to rt. Then the mixture was stirred for 1 h at rt, then it was quenched with sat. aq. NH₄Cl solution and extracted with THF. The combined organic layers were dried over Na₂SO₄, filtered and the filtrate was concentrated under reduced pressure. The crude intermediate was purified via prep RP-HPLC using 25 mM aq. NH₄HCO₃ solution and MeCN as eluents to obtain Example 361B. ¹H NMR (400 MHz, DMSO-d₆) δ ppm: 8.73 (d, 2H), 8.65 (s, 2H), 7.23-7.67 (m, 4H), 7.53-7.47 (m, 2H).

### Example 361 (1s,4s)-4-(3-chloroanilino)-2′-[4-(pyridin-4-yl)-1-benzofuran-2-yl]spiro[cyclohexane-1,1′-indene]-4-carboxylic acid

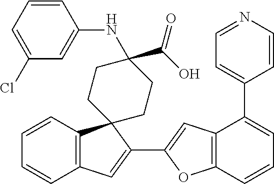 [SMILES: O=C(O)[C@]1(Nc2cccc(Cl)c2)CC[C@]2(CC1)C(c1cc3c(-c4ccncc4)cccc3o1)=Cc1ccccc12]

Using General procedure 18 and Preparation 6a as the appropriate 2-bromo-indene and Example 361B as the appropriate boronic acid, Example 361 was obtained. ¹H NMR (500 MHz, DMSO-d₆) δ ppm: 12.93 (br s, 1H), 8.85 (d, 2H), 7.96 (d, 2H), 7.83 (d, 1H), 7.75 (d, 1H), 7.59 (d, 1H), 7.58 (s, 1H), 7.54 (dd, 1H), 7.51 (t, 1H), 7.39 (t, 1H), 7.32 (t, 1H), 7.25 (s, 1H), 6.94 (t, 1H), 6.71 (br s, 1H), 6.58-6.52 (m, 3H), 2.60/1.16 (t+d, 4H), 2.50/2.29 (t+d, 4H). HRMS calculated for C₃₄H₂₇N₂O₃Cl: 546.1710; found: 547.1772 (M+H).

### Example 362 (1s,4s)-4-(3-chloroanilino)-2′-{3-[(pyridin-4-yl)methoxy]phenyl}spiro[cyclohexane-1,1′-indene]-4-carboxylic acid

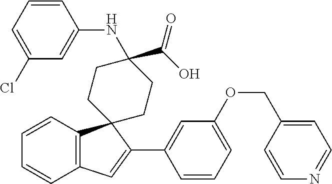 [SMILES: O=C(O)[C@]1(Nc2cccc(Cl)c2)CC[C@]2(CC1)C(c1cccc(OCc3ccncc3)c1)=Cc1ccccc12]

Using General procedure 32 and Preparation 12b as the appropriate indene and 4-pyridylmethanol as the appropriate alcohol, Example 362 was obtained. ¹H NMR (500 MHz, DMSO-d₆) δ ppm: 12.81 (br s, 1H), 8.59 (d, 2H), 7.78 (d, 1H), 7.47 (d, 2H), 7.45 (dd, 1H), 7.44 (d, 1H), 7.43 (d, 1H), 7.31 (t, 1H), 7.31 (t, 1H), 7.30 (s, 1H), 7.23 (t, 1H), 6.98 (t, 1H), 6.97 (d, 1H), 6.68 (dd, 1H), 6.62 (dd, 1H), 6.56 (dd, 1H), 6.55 (br s, 1H), 5.23 (s, 2H), 2.57/1.05 (t+d, 4H), 2.45/2.29 (t+d, 4H). HRMS calculated for C₃₃H₂₉N₂O₃Cl: 536.1866; found: 537.1934 (M+H).

### Example 363 (1s,4s)-4-(3-chloroanilino)-2′-{3-[(pyridin-3-yl)methoxy]phenyl}spiro[cyclohexane-1,1′-indene]-4-carboxylic acid

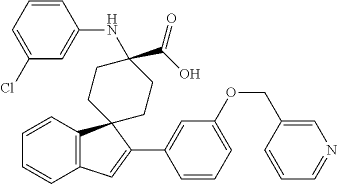 [SMILES: O=C(O)[C@]1(Nc2cccc(Cl)c2)CC[C@]2(CC1)C(c1cccc(OCc3cccnc3)c1)=Cc1ccccc12]

Using General procedure 32 and Preparation 12b as the appropriate indene and 3-pyridylmethanol as the appropriate alcohol, Example 363 was obtained. ¹H NMR (500 MHz, DMSO-d₆) δ ppm: 12.73 (br s, 1H), 8.72 (dd, 1H), 8.56 (dd, 1H), 7.90 (dt, 1H), 7.79 (d, 1H), 7.45 (dd, 1H), 7.44 (dd, 1H), 7.44 (d, 1H), 7.43 (d, 1H), 7.32 (t, 1H), 7.31 (t, 1H), 7.31 (s, 1H), 7.22 (t, 1H), 7.00 (dd, 1H), 6.92 (t, 1H), 6.64 (dd, 1H), 6.61 (dd, 1H), 6.56 (br s, 1H), 6.54 (dd, 1H), 5.20 (s, 2H), 2.57/1.04 (t+d, 4H), 2.45/2.27 (t+d, 4H). HRMS calculated for C₃₃H₂₉N₂O₃Cl: 536.1866; found: 537.1932 (M+H).

### Example 364 (1r,4r)-4-(3-chloroanilino)-2′-{4-[(pyridin-4-yl)oxy]phenyl}-2′,3′-dihydrospiro[cyclohexane-1,1′-indene]-4-carboxylic acid, enantiomer 1

and

### Example 365 (1r,4r)-4-(3-chloroanilino)-2′-{4-[(pyridin-4-yl)oxy]phenyl}-2′,3′-dihydrospiro[cyclohexane-1,1′-indene]-4-carboxylic acid, enantiomer 2

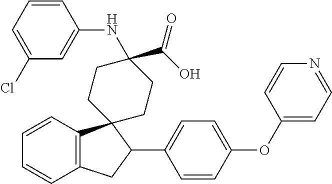

Using General procedure 19 and Example 342 as the appropriate indene and using AcOH instead of EtOAc, a racemate was obtained. The enantiomers were separated by chiral chromatography. Column: AD, 50×500 mm, 20 μm, eluents: EtOH+0.05% HCOOH. The enantiomer eluting earlier was collected as Example 364. ¹H NMR (500 MHz, DMSO-d₆) δ ppm: 12.70 (br, 1H), 8.43 (dm, 2H), 7.40-7.16 (m, 4H), 7.22 (m, 2H), 7.06 (d, 2H), 7.00 (t, 1H), 6.87 (dm, 2H), 6.52 (dm, 1H), 6.51 (m, 1H), 6.44 (dm, 1H), 6.08 (br, 1H), 3.44 (t, 1H), 3.31/3.11 (dd+dd, 2H), 2.45-1.28 (m, 8H). HRMS calculated for C₃₂H₂₉N₂O₃Cl: 524.1866; found: 525.1929 (M+H).

The enantiomer eluting later was collected as Example 365. HRMS calculated for C₃₂H₂₉N₂O₃Cl: 524.1866; found: 525.1928 (M+H).

### Example 366

### Example 366A methyl (1r,4r)-4-(3-chloroanilino)-6′-methoxy-2′-(3-methylphenyl)spiro[cyclohexane-1,1′-indene]-4-carboxylate

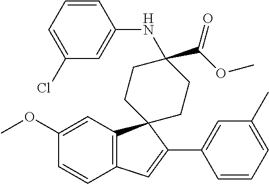

Using General procedure 18 and Preparation 18aI as the appropriate 2-bromo-indene and 4,4,5,5-tetramethyl-2-(m-tolyl)-1,3,2-dioxaborolane as the appropriate boronic ester, Example 366A was obtained. LRMS calculated for C₃₀H₃₀NO₃Cl: 487.2; found: 488.1 (M+H).

### Example 366 (1r,4r)-4-(3-chloroanilino)-6′-methoxy-2′-(3-methylphenyl)spiro[cyclohexane-1,1′-indene]-4-carboxylic acid

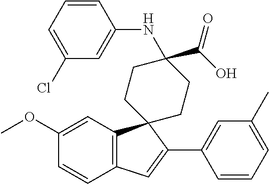

Using General procedure 33a and Example 366A as the appropriate ester, Example 366 was obtained. HRMS calculated for C₂₉H₂₈NO₃Cl: 473.1758; found: 474.1826 (M+H).

### Example 367 (1r,4r)-4-(3-chloroanilino)-2′-{4-[(pyridin-3-yl)oxy]phenyl}-2′,3′-dihydrospiro[cyclohexane-1,1′-indene]-4-carboxylic acid

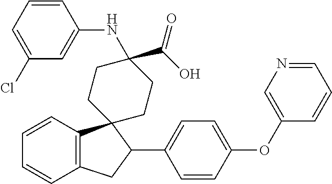

Using General procedure 19 and Example 351 as the appropriate indene and using AcOH instead of EtOAc, Example 367 was obtained as a racemate. HRMS calculated for C₃₂H₂₉N₂O₃Cl: 524.1866; found: 525.1928 (M+H).

### Example 368

### Example 368A (1r,4r)-4-(3-chloroanilino)-2′-(4-hydroxyphenyl)-2′,3′-dihydrospiro[cyclohexane-1,1′-indene]-4-carboxylic acid

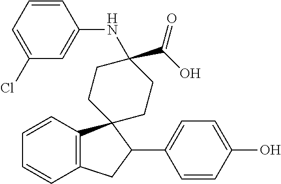

Using General procedure 19 and Example 349 as the appropriate indene and using AcOH instead of EtOAc, Example 368A was obtained as racemate. LRMS calculated for C₂₇H₂₆NO₃Cl: 447.2; found: 448.1 (M+H).

### Example 368B methyl (1r,4r)-4-(3-chloroanilino)-2′-(4-hydroxyphenyl)-2′,3′-dihydrospiro[cyclohexane-1,1′-indene]-4-carboxylate, enantiomer 1

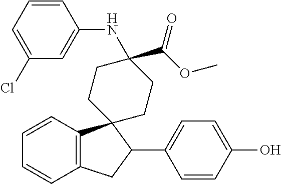

Using General procedure 17a and Example 368A as the appropriate amino acid a racemate was obtained. The enantiomers were separated by chiral chromatography. Column: AD, 50×500 mm, 20 μm, eluents: heptane/EtOH. The enantiomer eluting earlier was collected as Example 368B. LRMS calculated for C₂₈H₂₈NO₃Cl: 461.2; found: 462.1 (M+H).

### Example 368 (1r,4r)-4-(3-chloroanilino)-2′-{4-[(pyridin-3-yl)methoxy]phenyl}-2′,3′-dihydrospiro[cyclohexane-1,1′-indene]-4-carboxylic acid, enantiomer 1

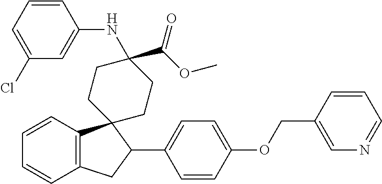

Using General procedure 32 and Example 368B as the appropriate indane and 3-pyridylmethanol as the appropriate alcohol, Example 368 was obtained. HRMS calculated for C₃₃H₃₁N₂O₃Cl: 538.2023; found: 539.2095 (M+H).

### Example 369 (1r,4r)-4-(3-chloroanilino)-2′-{4-[(pyridin-4-yl)methoxy]phenyl}-2′,3′-dihydrospiro[cyclohexane-1,1′-indene]-4-carboxylic acid, enantiomer 1

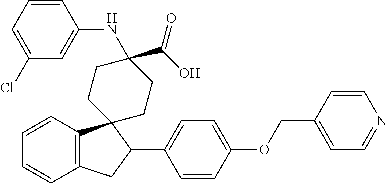

Using General procedure 32 and Example 368B as the appropriate indane and 4-pyridylmethanol as the appropriate alcohol, Example 369 was obtained. HRMS calculated for C₃₃H₃₁N₂O₃Cl: 538.2023; found: 539.2098 (M+H).

### Example 372 (1r,4r)-4-(3-chloroanilino)-2′-{3-[(pyridin-3-yl)methoxy]phenyl}-2′,3′-dihydrospiro[cyclohexane-1,1′-indene]-4-carboxylic acid, enantiomer 1

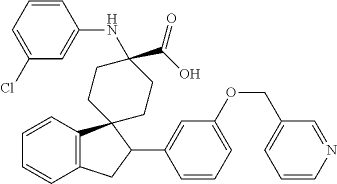

Using General procedure 32 and Preparation 12c as the appropriate ester, and 3-pyridylmethanol as the appropriate alcohol, Example 372 was obtained. ¹H NMR (500 MHz, DMSO-d₆) δ ppm: 12.66 (br s, 1H), 8.59 (d, 1H), 8.51 (dd, 1H), 7.76 (dm, 1H), 7.37 (m, 1H), 7.30-7.17 (m, 4H), 7.13 (t, 1H), 7.01 (t, 1H), 6.86 (dm, 1H), 6.68 (br s, 1H), 6.63 (dm, 1H), 6.52 (dm, 1H), 6.52 (t, 1H), 6.45 (dm, 1H), 6.01 (br s, 1H), 5.06/5.03 (d+d, 2H), 3.38 (m, 1H), 3.35/2.96 (m+m, 2H), 2.42-1.22 (m, 8H). HRMS calculated for C₃₃H₃₁N₂O₃Cl: 538.2023; found: 539.2091 (M+H).

### Example 373 (1r,4r)-4-(3-chloroanilino)-2′-{3-[(pyridin-4-yl)methoxy]phenyl}-2′,3′-dihydrospiro[cyclohexane-1,1′-indene]-4-carboxylic acid, enantiomer 1

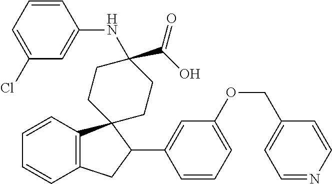

Using General procedure 32 and Preparation 12c as the appropriate ester, and 4-pyridylmethanol as the appropriate alcohol, Example 373 was obtained. ¹H NMR (500 MHz, DMSO-d₆) δ ppm: 12.65 (br s, 1H), 8.52 (m, 2H), 7.31 (m, 2H), 7.28-7.18 (m, 4H), 7.13 (t, 1H), 7.00 (t, 1H), 6.83 (dm, 1H), 6.66 (br s, 1H), 6.65 (m, 1H), 6.52 (t, 1H), 6.51 (dm, 1H), 6.45 (dm, 1H), 6.02 (br s, 1H), 5.09/5.05 (d+d, 2H), 3.38 (m, 1H), 3.35/2.96 (m+m, 2H), 2.42-1.20 (m, 8H). HRMS calculated for C₃₃H₃₁N₂O₃Cl: 538.2023; found: 539.2090 (M+H).

### Example 374 (1r,4r)-4-(3-chloroanilino)-2′-{3-[(pyridin-3-yl)oxy]phenyl}-2′,3′-dihydrospiro[cyclohexane-1,1′-indene]-4-carboxylic acid, enantiomer 1

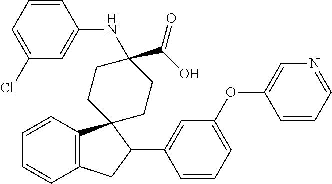

Preparation 12c (46 mg, 0.1 mmol) was dissolved in DMF (1 mL). 3-Iodopyridine (21 mg, 0.1 mmol), 2,2,6,6-tetramethylheptane-3,5-dione (9 mg, 0.05 mmol), CuI (1 mg, 0.005 mmol) and Cs₂CO₃ (49 mg, 0.15 mmol) were added. The mixture was stirred at 60° C. After 2 days all the reagents were added again in the same amount as at first time and the mixture was stirred until no further conversion was observed. The mixture was allowed to cool to rt, it was injected to column and was purified via flash chromatography using heptane and EtOAC as eluents. Then it was hydrolyzed according to General procedure 33a to obtain Example 374. ¹H NMR (500 MHz, DMSO-d₆) δ ppm: 12.6 (br s, 1H), 8.32 (dd, 1H), 8.29 (dd, 1H), 7.31-7.14 (m, 6H), 7.27 (t, 1H), 7.03 (t, 1H), 6.93 (br d, 1H), 6.88 (dm, 1H), 6.77 (br s, 1H), 6.53 (dm, 1H), 6.52 (t, 1H), 6.45 (dm, 1H), 6.04 (br s, 1H), 3.43 (dd, 1H), 3.33/3.03 (dd+dd, 2H), 2.43-1.26 (m, 8H). HRMS calculated for C₃₂H₂₉N₂O₃Cl: 524.1866; found: 525.1941 (M+H).

### Example 375

### Example 375A 4-(benzofuran-5-yl)pyridine

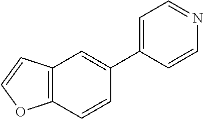 [SMILES: c1cc(-c2ccc3occc3c2)ccn1]

A microwave vial was charged with 5-bromobenzofuran (1.041 g, 5.28 mmol), 4-pyridylboronic acid (0.779 g, 6.34 mmol), Cs₂CO₃ (2.580 g, 7.92 mmol), dioxane (15 mL) and water (7 mL). The vial was purged with N₂, followed by addition of Ataphos (0.187 g, 0.264 mmol). The mixture was heated at 110° C. for 15 min under microwave irradiation. The mixture was allowed to cool to rt, it was injected directly to the column and purified via flash chromatography using heptane and EtOAc as eluents resulting Example 375A. ¹H NMR (400 MHz, DMSO-d₆) δ ppm: 8.63 (dd, 2H), 8.11 (m, 1H), 8.09 (d, 1H), 7.76-7.30 (m, 4H), 7.05 (d, 1H).

### Example 375B [5-(4-pyridyl)benzofuran-2-yl]boronic acid

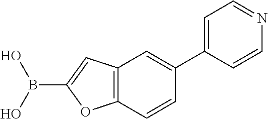 [SMILES: OB(O)c1cc2cc(-c3ccncc3)ccc2o1]

A solution of Example 375A (0.213 g, 1.09 mmol) in dry THE (5 mL) was cooled to −78° C. under N₂ atmosphere and then nBuLi in hexanes (2.5 M, 0.48 mL, 1.2 mmol) was added dropwise. After 30 minutes 2-isopropoxy-4,4,5,5-tetramethyl-1,3,2-dioxaborolane (0.255 mL, 1.25 mmol) was added dropwise. After 10 min the cooling bath was removed and the mixture was slowly allowed to warm up to rt. The reaction mixture was used in the next step as Example 375B without further work-up. LRMS calculated for C₁₃H₁₀BNO₃: 239.14; found: 240.2 (M+H).

### Example 375 (1s,4s)-4-(3-chloroanilino)-2′-[5-(pyridin-4-yl)-1-benzofuran-2-yl]spiro[cyclohexane-1,1′-indene]-4-carboxylic acid

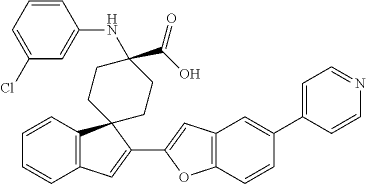 [SMILES: O=C(O)[C@]1(Nc2cccc(Cl)c2)CC[C@]2(CC1)C(c1cc3cc(-c4ccncc4)ccc3o1)=Cc1ccccc12]

Using General procedure 18 and Preparation 6a as the appropriate 2-bromo-indene and Example 375B as the appropriate boronic acid, Example 375 was obtained. HRMS calculated for C₃₄H₂₇N₂O₃Cl: 546.1710; found: 547.1770 (M+H).

### Example 376 (1r,4r)-4-(3-chloroanilino)-2′-{3-[(pyridin-4-yl)oxy]phenyl}-2′,3′-dihydrospiro[cyclohexane-1,1′-indene]-4-carboxylic acid, enantiomer 1

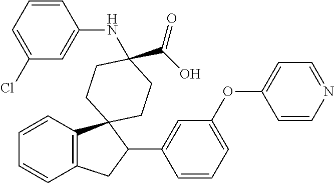

Preparation 12c (46 mg, 0.1 mmol) was dissolved in DMF (1 mL). 4-Iodopyridine (42 mg, 0.2 mmol), 2,2,6,6-tetramethylheptane-3,5-dione (18 mg, 0.1 mmol), CuI (2 mg, 0.01 mmol) and Cs₂CO₃ (98 mg, 0.3 mmol) were added. The mixture was stirred at 90° C. until no further conversion was observed. The mixture was allowed to cool to rt, it was injected to column and was purified via flash chromatography using heptane and EtOAC as eluents. Then it was hydrolyzed according to General procedure 33a to obtain Example 376. ¹H NMR (500 MHz, DMSO-d₆) δ ppm: 12.55 (br s, 1H), 8.30 (m, 2H), 7.36-7.15 (m, 4H), 7.35 (t, 1H), 7.05 (br d, 1H), 7.01 (dm, 1H), 7.01 (t, 1H), 6.89 (br s, 1H), 6.78 (m, 2H), 6.52 (t, 1H), 6.52 (dm, 1H), 6.44 (dm, 1H), 6.02 (br s, 1H), 3.44 (dd, 1H), 3.30/3.09 (dd+dd, 2H), 2.43-1.25 (m, 8H). HRMS calculated for C₃₂H₂₉N₂O₃Cl: 524.1866; found: 525.1943 (M+H).

### Example 377 (1s,4s)-4-(3-chloroanilino)-2′-{3-[(pyridin-4-yl)oxy]phenyl}spiro[cyclohexane-1,1′-indene]-4-carboxylic acid

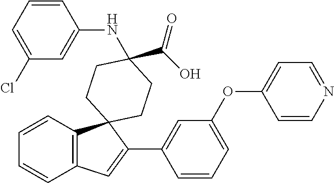 [SMILES: O=C(O)[C@]1(Nc2cccc(Cl)c2)CC[C@]2(CC1)C(c1cccc(Oc3ccncc3)c1)=Cc1ccccc12]

Preparation 12b (115 mg, 0.25 mmol) was dissolved in DMF (1 mL). 4-Iodopyridine (51 mg, 0.25 mmol), 2,2,6,6-tetramethylheptane-3,5-dione (23 mg, 0.125 mmol), CuI (3 mg, 0.013 mmol) and Cs₂CO₃ (122 mg, 0.375 mmol) were added. The mixture was stirred at 90° C. until no further conversion was observed. The mixture was allowed to cool to rt, it was diluted with water. The precipitation was filtered, washed with water and dried under reduced pressure. The crude product was purified via prep RP-HPLC using 25 mM aq. NH₄HCO₃ solution and MeCN as eluents. Then it was hydrolyzed according to General procedure 33a to obtain Example 377. HRMS calculated for C₃₂H₂₇N₂O₃Cl: 522.1710; found: 523.1785 (M+H).

### Example 378 (1r,4r)-4-(3-chloroanilino)-6′-methoxy-2′-(3-phenoxyphenyl)-2′,3′-dihydrospiro[cyclohexane-1,1′-indene]-4-carboxylic acid, enantiomer 1

and

### Example 379 (1r,4r)-4-(3-chloroanilino)-6′-methoxy-2′-(3-phenoxyphenyl)-2′,3′-dihydrospiro[cyclohexane-1,1′-indene]-4-carboxylic acid, enantiomer 2

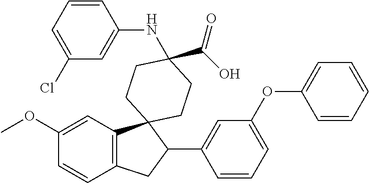

Using General procedure 33a and Preparation 18aK as the appropriate ester, Example 378 was obtained. ¹H NMR (500 MHz, DMSO-d₆) δ ppm: 12.57 (br s, 1H), 7.24 (m, 2H), 7.23 (t, 1H), 7.15 (d, 1H), 7.03 (m, 1H), 7.02 (t, 1H), 6.87 (m, 2H), 6.86 (dm, 1H), 6.81 (dm, 1H), 6.79 (d, 1H), 6.75 (dd, 1H), 6.71 (br s, 1H), 6.54 (t, 1H), 6.52 (dm, 1H), 6.46 (dm, 1H), 6.03 (br s, 1H), 3.73 (s, 3H), 3.41 (dd, 1H), 3.26/2.90 (dd+dd, 2H), 2.41-1.25 (m, 8H). HRMS calculated for C₃₄H₃₂NO₄Cl: 553.2020; found: 554.2083 (M+H).

Using General procedure 33a and Preparation 18aL as the appropriate ester, Example 379 was obtained. HRMS calculated for C₃₄H₃₂NO₄Cl: 553.2020; found: 554.2091 (M+H).

### Example 384 (1r,4r)-4-(3-chloroanilino)-6′-(2-methoxyethoxy)-2′-(3-phenoxyphenyl)-2′,3′-dihydrospiro[cyclohexane-1,1′-indene]-4-carboxylic acid, enantiomer 1

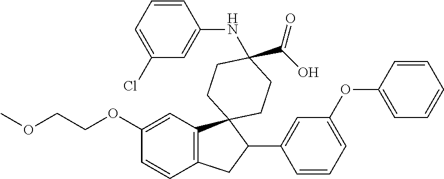

Using General procedure 32 and Preparation 18a as the appropriate indane and 2-methoxyethanol as the appropriate alcohol, Example 384 was obtained. ¹H NMR (500 MHz, DMSO-d₆) δ ppm: 12.70 (br s, 1H), 7.23 (m, 2H), 7.23 (t, 1H), 7.13 (d, 1H), 7.04 (m, 1H), 7.03 (dm, 1H), 7.03 (t, 1H), 6.87 (m, 2H), 6.81 (dm, 1H), 6.79 (d, 1H), 6.75 (dd, 1H), 6.71 (br s, 1H), 6.53 (t, 1H), 6.53 (dm, 1H), 6.45 (dm, 1H), 6.06 (br s, 1H), 4.05 (t, 2H), 3.66 (t, 2H), 3.39 (dd, 1H), 3.32 (s, 3H), 3.25/2.91 (dd+dd, 2H), 2.40-1.25 (m, 8H). HRMS calculated for C₃₆H₃₆NO₅Cl: 597.2282; found: 598.2353 (M+H).

### Example 385 (1r,4r)-4-(3-chloroanilino)-6′-(2-methylpropoxy)-2′-(3-phenoxyphenyl)-2′,3′-dihydrospiro[cyclohexane-1,1′-indene]-4-carboxylic acid, enantiomer 1

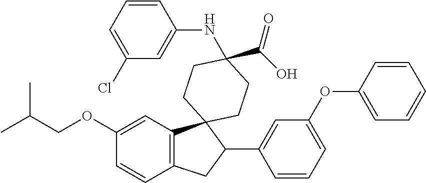

Using General procedure 32 and Preparation 18a as the appropriate indane and 2-methylpropan-1-ol as the appropriate alcohol, Example 385 was obtained. ¹H NMR (500 MHz, DMSO-d₆) δ ppm: 12.70 (br s, 1H), 7.23 (m, 2H), 7.23 (t, 1H), 7.12 (d, 1H), 7.03 (t, 1H), 7.03 (m, 1H), 6.87 (dm, 1H), 6.87 (m, 2H), 6.81 (dm, 1H), 6.77 (d, 1H), 6.73 (dd, 1H), 6.71 (br s, 1H), 6.54 (t, 1H), 6.53 (dm, 1H), 6.47 (dm, 1H), 6.07 (br s, 1H), 3.71/3.69 (m+m, 2H), 3.41 (dd, 1H), 3.26/2.89 (dd+dd, 2H), 2.41-1.25 (m, 8H), 2.01 (m, 1H), 0.99 (d, 6H). HRMS calculated for C₃₇H₃₈NO₄Cl: 595.2490; found: 596.2562 (M+H).

### Example 386 (1r,4r)-4-(3-chloroanilino)-6′-ethoxy-2′-(3-phenoxyphenyl)-2′,3′-dihydrospiro[cyclohexane-1,18′-indene]-4-carboxylic acid, enantiomer 1

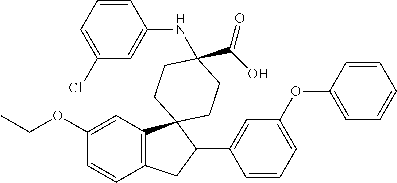

Using General procedure 32 and Preparation 18a as the appropriate indane and EtOH as the appropriate alcohol, Example 386 was obtained. HRMS calculated for C₃₅H₃₄NO₄Cl: 567.2177; found: 568.2267 (M+H).

### Example 387 (1r,4r)-4-(3-chloroanilino)-2′-(3-phenoxyphenyl)-6′-[(propan-2-yl)oxy]-2′,3′-dihydrospiro[cyclohexane-1,1′-indene]-4-carboxylic acid, enantiomer 1

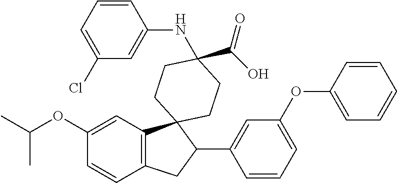

Using General procedure 32 and Preparation 18a as the appropriate indane and iPrOH as the appropriate alcohol, Example 387 was obtained. ¹H NMR (500 MHz, DMSO-d₆) δ ppm: 7.24 (t, 1H), 7.23 (m, 2H), 7.12 (d, 1H), 7.03 (m, 1H), 7.02 (t, 1H), 6.89 (br d, 1H), 6.87 (m, 2H), 6.82 (dm, 1H), 6.78 (d, 1H), 6.73 (br s, 1H), 6.73 (dd, 1H), 6.53 (dm, 1H), 6.52 (m, 1H), 6.44 (dm, 1H), 6.04 (br, 1H), 4.53 (sept., 1H), 3.38 (dd, 1H), 3.22/2.92 (dd+dd, 2H), 2.40-1.27 (m, 8H), 1.27/1.26 (d+d, 6H). HRMS calculated for C₃₆H₃₆NO₄Cl: 581.2333; found: 582.2407 (M+H).

### Example 393 (1r,4r)-4-(3-chloroanilino)-2′-(4-phenylbutyl)spiro[cyclohexane-1,1′-indene]-4-carboxylic acid

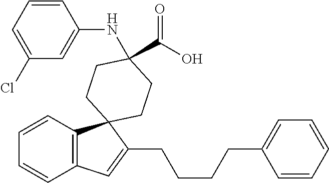 [SMILES: O=C(O)[C@]1(Nc2cccc(Cl)c2)CC[C@]2(CC1)C(CCCCc1ccccc1)=Cc1ccccc12]

To an oven-dried microwave vial was added Preparation 6a (110 mg, 0.25 mmol, 1 eq) and AtaPhos (4 mg, 0.01 mmol, 0.02 eq) in THE (2 mL). 1-methylimidazole (41 μL, 0.51 mmol, 2 eq) was added followed by 4-(phenyl)butylzinc chloride (1.02 mL, 0.5 M in THF, 0.51 mmol, 2 eq) and then heated at 110° C. for 1 h under microwave irradiation. The reaction was partitioned between DCM and water, and the organic phase was washed with 1 M aq. HCl solution, brine, dried (PTFE phase separator) and concentrated in vacuo. Purification by reverse phase automated flash chromatography (CombiFlash Rf, C18 13 g RediSep column) eluting with a gradient of 15-100% MeCN in water afforded Example 393 as a white powder (51 mg, 0.1 mmol, 41%). LRMS calculated for C₃₁H₃₂ClNO₂: 485; found: 486 (M+H). ¹H NMR (400 MHz, DMSO-d₆) δ ppm: 12.79 (br s, 1H), 7.66 (d, J=7.6 Hz, 1H), 7.30-7.13 (m, 7H), 7.13-7.06 (m, 2H), 6.66 (t, J=2.1 Hz, 1H), 6.60-6.55 (m, 2H), 6.41 (s, 1H), 6.33 (br s, 1H), 2.64 (t, J=7.0 Hz, 2H), 2.45-2.34 (m, 2H), 2.28-2.07 (m, 6H), 1.74-1.59 (m, 4H), 0.91-0.82 (m, 2H).

### Example 394 (1r,4r)-4-(3-chloroanilino)-2′-(4-phenylbutyl)-2′,3′-dihydrospiro[cyclohexane-1,1′-indene]-4-carboxylic acid

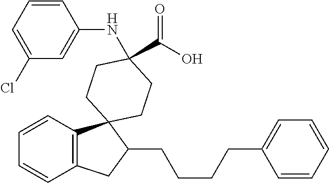

To a solution of Example 393 (97 mg, 0.2 mmol, 1 eq) in EtOH (3 mL) and THF (3 mL) was added Pt/C (39 mg, 0.01 mmol, 0.05 eq) under a N₂ atmosphere. The mixture was evacuated and backfilled with N₂ (×3), then evacuated and backfilled with H₂ and shaken at rt for 48 h under an atmosphere of H₂. The reaction was filtered through celite, eluted with EtOH and then concentrated under reduced pressure. Purification by automated flash chromatography (CombiFlash Rf, 4 g RediSep™ silica cartridge) eluting with a gradient of 0-40% EtOAc in heptane, followed by purification by preparative HPLC at pH 9 afforded the racemate Example 394 as a beige solid (4.4 mg, 0.01 mmol, 5%). LRMS calculated for C₃₁H₃₄ClNO₂: 487; found: 488 (M+H). ¹H NMR (400 MHz, DMSO-d₆) δ ppm: 7.40-7.33 (m, 1H), 7.30-7.24 (m, 2H), 7.22-7.10 (m, 6H), 7.03 (t, J=8.1 Hz, 1H), 6.65 (t, J=2.1 Hz, 1H), 6.58-6.49 (m, 2H), 6.16 (br s, 1H), 2.93 (dd, J=15.7, 7.2 Hz, 1H), 2.67-2.52 (m, 2H), 2.45-2.34 (m, 1H), 2.16-2.05 (m, 1H), 2.01-1.78 (m, 4H), 1.77-1.10 (m, 9H).

### Example 395 (1r,4r)-2′-[2-(benzyloxy)ethyl]-4-(3-chloroanilino)spiro[cyclohexane-1,1′-indene]-4-carboxylic acid

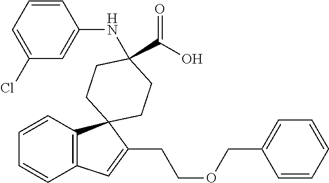 [SMILES: O=C(O)[C@]1(Nc2cccc(Cl)c2)CC[C@]2(CC1)C(CCOCc1ccccc1)=Cc1ccccc12]

To an oven-dried flask was added Preparation 8a (100 mg, 0.24 mmol, 1 eq) in THE (4 mL). NaH (60% dispersion; 29 mg, 0.73 mmol, 3 eq) was added portionwise and the mixture was allowed to stir for 5 min at 0° C. BnBr (87 μL, 0.73 mmol, 3 eq) was added and the mixture was stirred at rt for 48 h. The reaction was quenched with water, extracted with DCM, and the combined organic extracts were washed with brine, dried (PTFE phase separator) and concentrated in vacuo. To a solution of the intermediate in MeOH (1 mL) and THE (1 mL) was added LiOH×H₂O (82 mg, 1.94 mmol, 8 eq) and the mixture was heated at 120° C. for 40 min under microwave irradiation. The reaction was allowed to cool to rt and partitioned between DCM and water, and the organic phase was washed with brine, dried (PTFE phase separator) and concentrated in vacuo. Purification by automated flash chromatography (CombiFlash Rf, 4 g RediSep™ silica cartridge) eluting with a gradient of 0-50% EtOAc in heptane afforded a solid, which was triturated in heptane and Et₂O. The solids were collected by filtration and dried under vacuum to afford Example 395 as a white powder (32.4 mg, 0.07 mmol, 27%). LRMS calculated for C₃₀H₃₀ClNO₃: 487; found: 488 (M+H). ¹H NMR (400 MHz, DMSO-d₆) δ ppm: 12.82 (br s, 1H), 7.66 (d, J=7.6 Hz, 1H), 7.37-7.25 (m, 6H), 7.23 (td, J=7.4, 0.9 Hz, 1H), 7.15-7.07 (m, 2H), 6.66 (t, J=2.1 Hz, 1H), 6.61-6.56 (m, 2H), 6.49 (s, 1H), 6.37 (br s, 1H), 4.54 (s, 2H), 3.74 (t, J=7.0 Hz, 2H), 2.45-2.33 (m, 2H), 2.24-2.08 (m, 4H), 0.93-0.84 (m, 2H).

### Example 396 (1r,4r)-2′-[2-(benzyloxy)ethyl]-4-(3-chloroanilino)-2′,3′-dihydrospiro[cyclohexane-1,1′-indene]-4-carboxylic acid

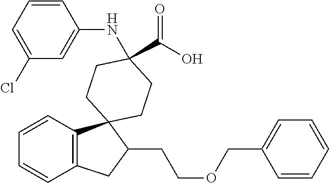

To an oven-dried flask was added Preparation 8bA (134 mg, 0.32 mmol, 1 eq) in THE (4 mL). NaH (60% dispersion; 23 mg, 0.97 mmol, 3 eq) was added and the mixture was stirred for 5 min before the addition of BnBr (116 μL, 0.97 mmol, 3 eq). The mixture was stirred at rt for 18 h and then partitioned between DCM and water. The combined organic extracts were washed with brine, dried (MgSO₄) and concentrated in vacuo. To the intermediate dissolved in MeOH (3 mL), THF (0.5 mL) and water (0.3 mL) was added LiOH×H₂O (109 mg, 2.59 mmol, 8 eq) and the mixture was heated at 110° C. for 1 h under microwave irradiation. The reaction was allowed to cool to rt and partitioned between DCM and water, acidified with 2 M aq. HCl solution, and the organic phase was washed with brine, dried (MgSO₄) and concentrated in vacuo. Purification by automated flash chromatography (CombiFlash Rf, 4 g RediSep™ silica cartridge) eluting with a gradient of 0-50% EtOAc in heptane afforded a solid. The solid was dissolved in minimal DCM, then loaded onto a DCM-wet PE-AX cartridge (5 g), washed successively with DCM, MeOH and eluted with 5% HCOOH in DCM, and concentrated in vacuo to afford a racemic mixture Example 396 as a white solid (28.4 mg, 0.06 mmol, 18%). LRMS calculated for C₃₀H₃₂ClNO₃: 489; found: 490 (M+H). ¹H NMR (400 MHz, DMSO-d₆) δ ppm: 12.69 (br s, 1H), 7.42-7.25 (m, 6H), 7.22-7.11 (m, 3H), 7.07 (t, J=8.1 Hz, 1H), 6.63 (t, J=2.1 Hz, 1H), 6.58-6.52 (m, 2H), 6.25 (br s, 1H), 4.51 (d, J=12.1 Hz, 1H), 4.46 (d, J=12.1 Hz, 1H), 3.61-3.47 (m, 2H), 2.92 (dd, J=15.6, 7.3 Hz, 1H), 2.60-2.52 (m, 1H), 2.47-2.37 (m, 1H), 2.14-2.03 (m, 2H), 2.03-1.80 (m, 4H), 1.77-1.67 (m, 1H), 1.51-1.27 (m, 3H).

### Example 397

### Example 397A methyl (1r,4r)-4-(3-chloroanilino)-2′-[3-(phenylsulfanyl)propyl]-2′,3′-dihydrospiro[cyclohexane-1,1′-indene]-4-carboxylate

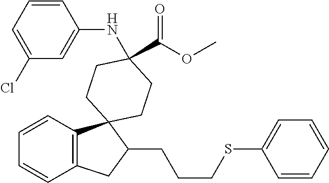

To a solution of Preparation 9b (83 mg, 0.19 mmol, 1 eq) in DCM (2 mL) was added thiophenol (24 μL, 0.23 mmol, 1.2 eq) and PPh₃ (102 mg, 0.39 mmol, 2 eq) followed by DIAD (77 μL, 0.39 mmol, 2 eq) and the mixture was stirred at rt for 18 h. The reaction was concentrated in vacuo and purification by automated flash column chromatography (CombiFlash Rf, 24 g RediSep™ silica cartridge) eluting with a gradient of 0-20% MeOH in DCM afforded racemate Example 397A as a colorless solid (15 mg, 0.03 mmol, 15%). LRMS calculated for C₃1H₃₄ClNO₂S: 519; found: 520 (M+H). ¹H NMR (400 MHz, CD₃OD) δ ppm: 7.43-7.37 (m, 1H), 7.37-7.32 (m, 2H), 7.31-7.24 (m, 2H), 7.21-7.10 (m, 4H), 7.06 (t, J=8.0 Hz, 1H), 6.67-6.60 (m, 2H), 6.56-6.51 (m, 1H), 3.73 (s, 3H), 3.08-2.92 (m, 3H), 2.60-2.50 (m, 2H), 2.34-2.24 (m, 1H), 2.13-1.44 (m, 10H), 1.42-1.27 (m, 1H).

### Example 397 (1r,4r)-4-(3-chloroanilino)-2′-[3-(phenylsulfanyl)propyl]-2′,3′-dihydrospiro[cyclohexane-1,1′-indene]-4-carboxylic acid

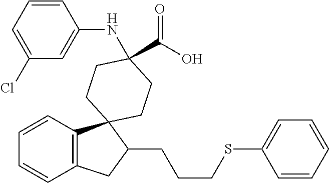

To a solution of Example 397A (15 mg, 0.03 mmol, 1 eq) in MeOH (3 mL) was added a solution of LiOH×H₂O (12.1 mg, 0.29 mmol, 10 eq) in water (0.3 mL) and the mixture was heated at 110° C. for 2 h under microwave irradiation. The mixture was allowed to cool to rt and concentrated in vacuo. The solid was dissolved in MeOH, then loaded onto a MeOH-wet PE-AX cartridge, washed successively with MeOH and DCM, eluted with 10% HCOOH in DCM, and concentrated in vacuo to afford racemic mixture Example 397 as an off-white solid (14.7 mg, 0.03 mmol, 100%). LRMS calculated for C₃₀H₃₂ClNO₂S: 505; found: 506 (M+H). ¹H NMR (400 MHz, DMSO-d₆) δ ppm: 7.38-7.27 (m, 5H), 7.22-7.11 (m, 4H), 7.07 (t, J=8.1 Hz, 1H), 6.64 (t, J=2.1 Hz, 1H), 6.58-6.53 (m, 2H), 6.21 (br s, 1H), 3.09-2.89 (m, 3H), 2.46-2.35 (m, 1H), 2.18-2.08 (m, 1H), 2.04-1.92 (m, 2H), 1.92-1.80 (m, 2H), 1.78-1.53 (m, 4H), 1.50-1.34 (m, 2H), 1.33-1.21 (m, 1H).

### Example 398 (1r,4r)-2′-[3-(benzenesulfonyl)propyl]-4-(3-chloroanilino)-2′,3′-dihydrospiro[cyclohexane-1,1′-indene]-4-carboxylic acid

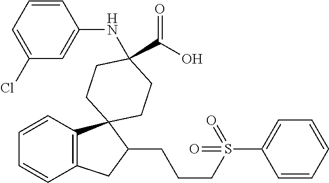

To a solution of Example 397A (8.2 mg, 0.02 mmol, 1 eq) in DCM (2 mL) was added mCPBA (8 mg, 0.05 mmol, 3 eq) and the mixture was stirred at rt for 45 min. The mixture was partitioned between DCM and sat. aq. K₂CO₃ solution, the aq. phase was extracted with DCM and the organic phase was washed with water, brine, dried (MgSO₄) and concentrated in vacuo. The intermediate was stirred in MeOH (2 mL) and a solution of LiOH×H₂O (7 mg, 0.16 mmol, 10 eq) in water (0.2 mL) was added and the mixture was heated at 110° C. for 30 min under microwave irradiation. The reaction was partitioned between DCM and water, acidified to pH 3 with 2 N aq. HCl solution and the organic phase was washed with brine, dried (MgSO₄) and concentrated in vacuo. Purification by preparative HPLC at pH 9 afforded racemic mixture Example 398 as a white solid (1.35 mg, 2.51 μmol, 16%). LRMS calculated for C₃₀H₃₂ClNO₄S: 537; found: 538 (M+H). ¹H NMR (400 MHz, DMSO-d₆) δ ppm: 12.68 (br s, 1H), 7.93-7.86 (m, 2H), 7.77-7.70 (m, 1H), 7.69-7.62 (m, 2H), 7.35-7.28 (m, 1H), 7.22-7.10 (m, 3H), 7.06 (t, J=8.1 Hz, 1H), 6.62 (t, J=2.1 Hz, 1H), 6.59-6.51 (m, 2H), 6.17 (br s, 1H), 2.89 (dd, J=15.6, 7.1 Hz, 1H), 2.49-2.32 (m, 2H), 2.17-2.07 (m, 1H), 2.02-1.89 (m, 2H), 1.87-1.33 (m, 8H), 1.27-1.12 (m, 1H).

### Example 399

### Example 399A methyl (1r,4r)-4-(3-chloroanilino)-2′-{3-[(pyridin-4-yl)sulfanyl]propyl}-2′,3′-dihydrospiro[cyclohexane-1,1′-indene]-4-carboxylate

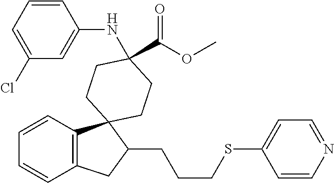

To a solution of Preparation 9c (32 mg, 0.06 mmol, 1 eq) in DCM (2 mL) was added 4-thiopyridine (10 mg, 0.09 mmol, 1.4 eq) and DIPEA (99 μL, 0.57 mmol, 9 eq). The mixture was stirred at rt for 18 h. The reaction was partitioned between DCM and water, and the organic phase was washed with water, brine, dried (MgSO₄) and concentrated in vacuo. Purification by automated flash column chromatography (CombiFlash Rf, 12 g RediSep™ silica cartridge) eluting with a gradient of 0-5% MeOH in DCM afforded racemate Example 399A as a solid (17 mg, 0.03 mmol, 52%). LRMS calculated for C₃₀H₃₃ClN₂O₂S: 520; found: 521 (M+H). ¹H NMR (400 MHz, CD₃OD) δ ppm: 8.46-8.42 (m, 2H), 7.51-7.47 (m, 2H), 7.45-7.37 (m, 1H), 7.22-7.10 (m, 3H), 7.05 (t, J=8.1 Hz, 1H), 6.67-6.60 (m, 2H), 6.55-6.50 (m, 1H), 3.73 (s, 3H), 3.20-2.99 (m, 3H), 2.68-2.49 (m, 2H), 2.35-2.24 (m, 1H), 2.16-2.05 (m, 2H), 2.05-1.28 (m, 9H).

### Example 399 (1r,4r)-4-(3-chloroanilino)-2′-{3-[(pyridin-4-yl)sulfanyl]propyl}-2′,3′-dihydrospiro[cyclohexane-1,1′-indene]-4-carboxylic acid

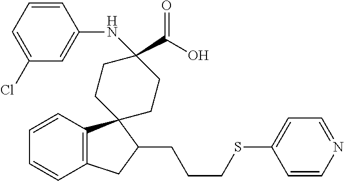

To a solution of Example 399A (17 mg, 0.03 mmol, 1 eq) in MeOH (3 mL) was added 1 M aq. NaOH solution (0.1 mL, 0.1 mmol, 3 eq) and the mixture was heated at 120° C. for 1.5 h under microwave irradiation. The mixture was concentrated in vacuo and suspended in a minimum amount of water, acidified with 2 N aq. HCl solution and the organics were extracted with DCM. The organic phase was washed with brine, dried (MgSO₄) and concentrated in vacuo.

Purification by preparative HPLC at pH 4 afforded racemic mixture Example 399 as a white solid. LRMS calculated for C₂₉H₃₁ClN₂O₂S: 506; found: 507 (M+H). ¹H NMR (400 MHz, DMSO-d₆) δ ppm: 8.38-8.32 (m, 2H), 7.38-7.33 (m, 1H), 7.28-7.24 (m, 2H), 7.18-7.07 (m, 3H), 6.91 (t, J=8.0 Hz, 1H), 6.71 (t, J=2.1 Hz, 1H), 6.62-6.57 (m, 1H), 6.36 (dd, J=7.4, 2.0 Hz, 1H), 5.65 (s, 1H), 3.16-2.91 (m, 3H), 2.43-2.33 (m, 1H), 2.18-2.08 (m, 1H), 2.08-1.98 (m, 1H), 1.81-1.43 (m, 9H), 1.30-1.18 (m, 1H).

### Example 400

### Example 400A (1s,4s)-4-(3-chloroanilino)-2′-{[(2E)-3-phenylprop-2-en-1-yl]oxy}-2′,3′-dihydrospiro[cyclohexane-1,1′-indene]-4-carboxylic acid

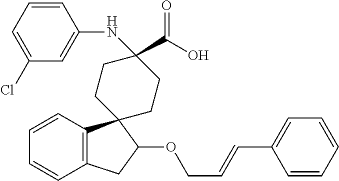

To a solution of Example 122 (25 mg, 0.06 mmol, 1 eq) in MeCN (2 mL), was added iodobenzene (14 μL, 0.12 mmol, 2 eq), TEA (34 μL, 0.24 mmol, 4 eq) and tri-o-tolylphosphine (7 mg, 0.02 mmol, 0.4 eq) and the mixture was sparged with N₂ for 15 min. Pd(OAc)₂ (3 mg, 0.01 mmol, 0.2 eq) was added and the reaction was heated at 120° C. for 1 h under microwave irradiation. The mixture was filtered through celite and the organics eluted with DCM. The combined organic extracts were concentrated in vacuo. Purification by automated flash chromatography (CombiFlash Rf, 4 g RediSep™ silica cartridge) eluting with a gradient of 0-100% EtOAc in heptane afforded racemate Example 400A as a brown solid (22.3 mg, 0.05 mmol, 75%). LRMS calculated for C₃₀H₃₀NO₃Cl: 487; found: 488 (M+H).

### Example 400 (1s,4s)-4-(3-chloroanilino)-2′-(3-phenylpropoxy)-2′,3′-dihydrospiro[cyclohexane-1,1′-indene]-4-carboxylic acid

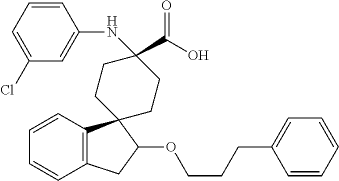

Example 400A (22.3 mg, 0.05 mmol, 1 eq) was dissolved in EtOH (5 mL) and 5% Pt/C (50 mg, 0.26 mmol, 5.61 eq) was added under a N₂ atmosphere. The mixture was evacuated and backfilled with N₂ (×3), then evacuated and backfilled with H₂ and shaken at rt for 18 h under an atmosphere of H₂. The mixture was filtered through celite and concentrated in vacuo. Purification by automated flash chromatography (CombiFlash Rf, 4 g RediSep™ silica cartridge) eluting with a gradient of 0-100% EtOAc in heptane, followed by preparative HPLC at pH 9 and then pH 4 afforded racemate Example 400 as a colourless solid (1.47 mg, 3 μmol, 7%). LRMS calculated for C₃₀H₃₂NO₃Cl: 489; found: 490 (M+H). ¹H NMR (400 MHz, DMSO-d₆) δ ppm: 7.29-7.11 (m, 9H), 7.03 (t, J=8.2 Hz, 1H), 6.69 (s, 1H), 6.63-6.57 (m, 1H), 6.55-6.50 (m, 1H), 3.97-3.90 (m, 1H), 3.64-3.55 (m, 1H), 3.08 (dd, J=16.5, 5.5 Hz, 1H), 2.87-2.78 (m, 1H), 2.71-2.56 (m, 2H), 2.45-2.24 (m, 2H), 2.14-2.00 (m, 1H), 1.91-1.54 (m, 7H).

### Example 401

### Example 401A (1s,4s)-4-(3-chloroanilino)-2′-{[(2E)-3-(3-methylpyridin-4-yl)prop-2-en-1-yl]oxy}-2′,3′-dihydrospiro[cyclohexane-1,1′-indene]-4-carboxylic acid

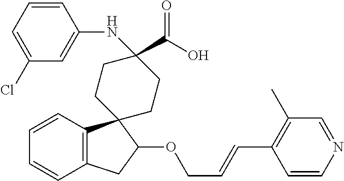

To a solution of Example 122 (25 mg, 0.06 mmol, 1 eq) in MeCN (2 mL), was added 4-bromo-3-methylpyridine×HCl (25 mg, 0.12 mmol, 2 eq), TEA (34 μL, 0.24 mmol, 4 eq) and tri-o-tolylphosphine (7 mg, 0.02 mmol, 0.4 eq) and the mixture was sparged with N₂ for 15 min. Pd(OAc)₂ (3 mg, 0.01 mmol, 0.2 eq) was added and the reaction was heated at 120° C. for 1 h under microwave irradiation. The reaction mixture was concentrated in vacuo and the residue was dissolved in a minimum amount of MeOH with a few drops of TEA. The material was loaded onto a MeOH-wet PE-AX cartridge (10 g), washed successively with MeOH, DCM and eluted with 10% HCOOH in DCM, then concentrated in vacuo to afford racemate Example 401A as a solid (26.3 mg, 0.05 mmol, 86%). LRMS calculated for C₃₀H₃₁N₂O₃Cl: 502; found: 503 (M+H).

### Example 401 (1s,4s)-4-(3-chloroanilino)-2′-[3-(3-methylpyridin-4-yl)propoxy]-2′,3′-dihydrospiro[cyclohexane-1,1′-indene]-4-carboxylic acid

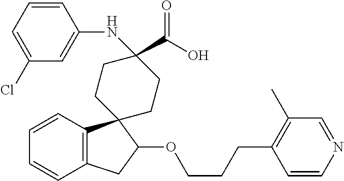

Example 401A (26 mg, 0.05 mmol, 1 eq) was dissolved in EtOH (5 mL) and 5% Pt/C (35 mg, 0.18 mmol, 3.47 eq) was added under a N₂ atmosphere. The mixture was evacuated and backfilled with N₂ (×3), then evacuated and backfilled with H₂ and shaken at rt for 18 h under an atmosphere of H₂. The mixture was filtered through celite and concentrated in vacuo. Purification by automated flash chromatography (CombiFlash Rf, 4 g RediSep™ silica cartridge) eluting with a gradient of 0-4% MeOH in DCM, followed by further automated flash chromatography (CombiFlash Rf, 12 g RediSep™ silica cartridge) eluting with a gradient of 0-4% MeOH in DCM afforded racemic mixture Example 401 as a colourless glass (4.4 mg, 0.01 mmol, 17%). LRMS calculated for C₃₀H₃₃N₂O₃Cl: 504; found: 505 (M+H). ¹H NMR (400 MHz, DMSO-d₆) δ ppm: 8.27 (br s, 2H), 7.27-7.08 (m, 5H), 7.04 (t, J=8.1 Hz, 1H), 6.66 (t, J=2.1 Hz, 1H), 6.60-6.52 (m, 2H), 3.96 (dd, J=5.4, 3.6 Hz, 1H), 3.69-3.59 (m, 1H), 3.09 (dd, J=16.3, 5.5 Hz, 1H), 2.84 (dd, J=16.4, 3.6 Hz, 1H), 2.74-2.54 (m, 2H), 2.45-2.36 (m, 1H), 2.32-2.23 (m, 1H), 2.18 (s, 3H), 2.12-2.02 (m, 1H), 1.91-1.52 (m, 7H).

### Example 402

### Example 402A methyl (1s,4s)-4-(3-chloroanilino)-2′-{[2-(pyridin-4-yl)ethyl]carbamoyl}spiro[cyclohexane-1,1′-indene]-4-carboxylate

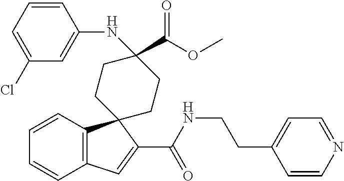 [SMILES: COC(=O)[C@]1(Nc2cccc(Cl)c2)CC[C@]2(CC1)C(C(=O)NCCc1ccncc1)=Cc1ccccc12]

Using General procedure 21 and Preparation 7a as the appropriate carboxylic acid and 2-(4-pyridyl)ethanamine as the appropriate amine Example 402A was obtained. ¹H NMR (500 MHz, DMSO-d₆) δ ppm: 8.42 (d, 2H), 8.29 (t, 1H), 7.72 (d, 1H), 7.50 (d, 1H), 7.33 (t, 1H), 7.31 (s, 1H), 7.30 (t, 1H), 7.24 (d, 2H), 7.09 (t, 1H), 6.63 (dd, 1H), 6.58 (dd, 1H), 6.46 (dd, 1H), 6.29 (s, 1H), 3.68 (s, 3H), 3.44 (q, 2H), 2.83 (t, 2H), 2.82/0.94 (t+d, 4H), 2.36/2.21 (t+d, 4H). HRMS calculated for C₃₀H₃₀N₃O₃Cl: 516.2054; found: 516.2034 (M+H).

### Example 402 (1s,4s)-4-(3-chloroanilino)-2′-{[2-(pyridin-4-yl)ethyl]carbamoyl}spiro[cyclohexane-1,1′-indene]-4-carboxylic acid

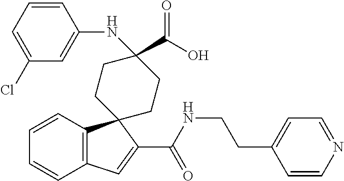 [SMILES: O=C(NCCc1ccncc1)C1=Cc2ccccc2[C@]12CC[C@@](Nc1cccc(Cl)c1)(C(=O)O)CC2]

Using General procedure 33a and Example 402A as the appropriate ester Example 402 was obtained. ¹H NMR (500 MHz, DMSO-d₆) δ ppm: 12.80 (br s, 1H), 8.43 (d, 2H), 8.28 (t, 1H), 7.73 (d, 1H), 7.49 (d, 1H), 7.33 (t, 1H), 7.30 (s, 1H), 7.29 (t, 1H), 7.24 (d, 2H), 7.08 (t, 1H), 6.64 (dd, 1H), 6.56 (dd, 1H), 6.55 (dd, 1H), 6.16 (br s, 1H), 3.44 (q, 2H), 2.83 (t, 2H), 2.80/0.93 (t+d, 4H), 2.35/2.18 (t+d, 4H). HRMS calculated for C₂₉H₂₈N₃O₃Cl: 501.1819; found: 502.1887 (M+H).

### Example 403

### Example 403A methyl (1s,4s)-4-(3-chloroanilino)-2′-{methyl[2-(pyridin-4-yl)ethyl]carbamoyl}spiro[cyclohexane-1,1′-indene]-4-carboxylate

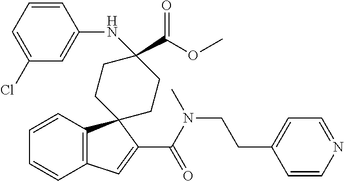 [SMILES: COC(=O)[C@]1(Nc2cccc(Cl)c2)CC[C@]2(CC1)C(C(=O)N(C)CCc1ccncc1)=Cc1ccccc12]

Using General procedure 21 and Preparation 7a as the appropriate carboxylic acid and N-methyl-2-(4-pyridyl)ethanamine as the appropriate amine Example 403A was obtained. ¹H NMR (500 MHz, DMSO-d₆) δ ppm: 8.43 (m, 2H), 7.67-7.21 (m, 4H), 7.23 (m, 2H), 7.06 (t, 1H), 6.64 (t, 1H), 6.58 (dm, 1H), 6.57 (br s, 1H), 6.50 (dm, 1H), 6.10 (s, 1H), 3.70 (s, 3H), 3.64 (t, 2H), 2.94 (s, 3H), 2.91 (t, 2H), 2.41/2.18 (m+m, 4H), 2.31/1.29 (m+m, 4H). HRMS calculated for C₃₁H₃₂N₃O₃Cl: 530.2210; found: 530.2208 (M+H).

### Example 403 (1s,4s)-4-(3-chloroanilino)-2′-{methyl[2-(pyridin-4-yl)ethyl]carbamoyl}spiro[cyclohexane-1,1′-indene]-4-carboxylic acid

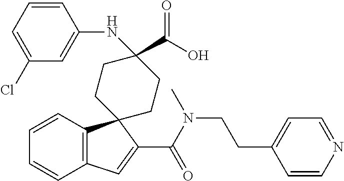 [SMILES: CN(CCc1ccncc1)C(=O)C1=Cc2ccccc2[C@]12CC[C@@](Nc1cccc(Cl)c1)(C(=O)O)CC2]

Using General procedure 33a and Example 403A as the appropriate ester Example 403 was obtained. ¹H NMR (500 MHz, DMSO-d₆) δ ppm: 11.62 (br s, 1H), 8.84 (m, 2H), 7.68-7.20 (m, 4H), 7.23 (m, 2H), 7.04 (t, 1H), 6.64 (t, 1H), 6.56 (dm, 1H), 6.56 (br s, 1H), 6.55 (dm, 1H), 6.29 (br s, 1H), 3.64 (br m, 2H), 2.93 (s, 3H), 2.90 (m, 2H), 2.40/2.14 (m+m, 4H), 2.28/1.28 (m+m, 4H). HRMS calculated for C₃₀H₃₀N₃O₃Cl: 515.1976; found: 516.2044 (M+H).

### Example 404

### Example 404A (1r,4r)-4-(3-chloroanilino)-4-(methoxycarbonyl)-2′,3′-dihydrospiro[cyclohexane-1,1′-indene]-2′-carboxylic acid, enantiomer 1

and

### Example 404B (1r,4r)-4-(3-chloroanilino)-4-(methoxycarbonyl)-2′,3′-dihydrospiro[cyclohexane-1,1′-indene]-2′-carboxylic acid, enantiomer 2

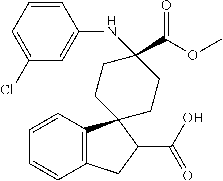

Using General procedure 19 and Preparation 7a as the appropriate indene (in AcOH/THF solvent mixture) a racemate was obtained. The enantiomers were separated by chiral chromatography. Column: IA, 100×500 mm, 20 μm, eluents: THF/EtOH/heptane. The enantiomer eluting earlier was collected as Example 404A. ¹H NMR (500 MHz, DMSO-d₆) δ ppm: 12.33 (br s, 1H), 7.29-7.14 (m, 4H), 7.06 (t, 1H), 6.58 (t, 1H), 6.56 (dm, 1H), 6.45 (dm, 1H), 6.34 (s, 1H), 3.65 (s, 3H), 3.06 (m, 2H), 3.02 (m, 1H) 2.46-1.50 (m, 8H). HRMS calculated for C₂₃H24NO₄Cl: 414.1472; found: 414.1483 (M+H).

The enantiomer eluting later was collected as Example 404B. HRMS calculated for C₂₃H₂₄NO₄Cl: 414.1472; found: 414.1463 (M+H).

### Example 404 (1r,4r)-4-(3-chloroanilino)-2′-{methyl[2-(pyridin-4-yl)ethyl]carbamoyl}-2′,3′-dihydrospiro[cyclohexane-1,1′-indene]-4-carboxylic acid, enantiomer 2

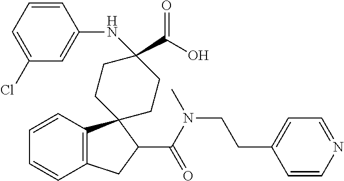

Using General procedure 21 and Example 404B as the appropriate carboxylic acid and N-methyl-2-(4-pyridyl)ethanamine as the appropriate amine an intermediate was obtained, which was hydrolysed according to General procedure 33a to obtain Example 404. HRMS calculated for C₃₀H₃₂N₃O₃Cl: 517.2132; found: 518.2204 (M+H).

### Example 405 (1r,4r)-4-(3-chloroanilino)-2′-[(2-phenylethyl)carbamoyl]-2′,3′-dihydrospiro[cyclohexane-1,1′-indene]-4-carboxylic acid, enantiomer 1

### Example 406 (1r,4r)-4-(3-chloroanilino)-2′-[(2-phenylethyl)carbamoyl]-2′,3′-dihydrospiro[cyclohexane-1,1′-indene]-4-carboxylic acid, enantiomer 2

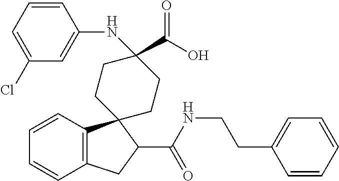

Using General procedure 21 and Example 404A as the appropriate carboxylic acid and 2-phenylethanamine as the appropriate amine an intermediate was obtained, which was hydrolysed according to General procedure 33a to obtain Example 405. ¹H NMR (500 MHz, DMSO-d₆) δ ppm: 8.08 (t, 1H), 7.27-7.08 (m, 9H), 6.97 (t, 1H), 6.65 (t, 1H), 6.55 (dm, 1H), 6.47 (dm, 1H), 6.07 (br s, 1H), 3.99/2.95 (dd+dd, 2H), 3.36/3.17 (m+m, 2H), 2.92 (dd, 1H), 2.77/2.70 (m+m, 2H), 2.44-1.55 (m, 8H). HRMS calculated for C₃₀H₃₁N₂O₃Cl: 502.2023; found: 503.2089 (M+H).

Using General procedure 21 and Example 404B as the appropriate carboxylic acid and 2-phenylethanamine as the appropriate amine an intermediate was obtained, which was hydrolysed according to General procedure 33a to obtain Example 406. HRMS calculated for C₃₀H₃₁N₂O₃Cl: 502.2023; found: 503.2098 (M+H).

### Example 407 (1r,4r)-4-(3-chloroanilino)-2′-[methyl(2-phenylethyl)carbamoyl]-2′,3′-dihydrospiro[cyclohexane-1,1′-indene]-4-carboxylic acid, enantiomer 1

and

### Example 408 (1r,4r)-4-(3-chloroanilino)-2′-[methyl(2-phenylethyl)carbamoyl]-2′,3′-dihydrospiro[cyclohexane-1,1′-indene]-4-carboxylic acid, enantiomer 2

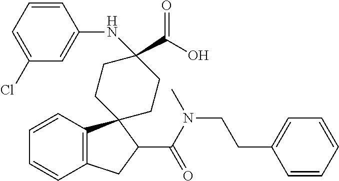

Using General procedure 21 and Example 404A as the appropriate carboxylic acid and N-methyl-2-phenyl-ethanamine as the appropriate amine an intermediate was obtained, which was hydrolysed according to General procedure 33a to obtain Example 407. ¹H NMR (500 MHz, DMSO-d₆) δ ppm: 12.32 (br s, 1H), 7.33-6.95 (m, 10H), 6.61 (t, 1H), 6.52 (dm, 1H), 6.51 (dm, 1H), 6.27 (br s, 1H), 3.95/3.71/3.50/3.28 (m+m, 2H), 3.50/3.20 (dd+dd, 1H), 3.11/2.83 (s, 3H), 3.01/2.96/2.64/2.50 (dd+dd, 2H), 2.86/2.76 (m, 2H), 2.43-1.51 (m, 8H). HRMS calculated for C₃₁H₃₃N₂O₃Cl: 516.2180; found: 517.2261 (M+H).

Using General procedure 21 and Example 404B as the appropriate carboxylic acid and N-methyl-2-phenyl-ethanamine as the appropriate amine an intermediate was obtained, which was hydrolysed according to General procedure 33a to obtain Example 408. HRMS calculated for C₃₁H₃₃N₂O₃Cl: 516.2180; found: 517.2267 (M+H).

### Example 409 (1s,4s)-4-(3-chloroanilino)-2′-[(2-phenylethoxy)methyl]spiro[cyclohexane-1,1′-indene]-4-carboxylic acid

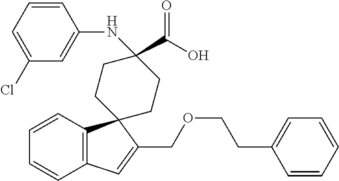 [SMILES: O=C(O)[C@]1(Nc2cccc(Cl)c2)CC[C@]2(CC1)C(COCCc1ccccc1)=Cc1ccccc12]

Using General procedure 20 and 2-phenylethanol as the appropriate alcohol Example 409 was obtained. ¹H NMR (500 MHz, DMSO-d₆) δ ppm: 12.79 (br s, 1H), 7.67-7.11 (m, 9H), 7.08 (t, 1H), 6.64 (t, 1H), 6.57 (dm, 1H), 6.55 (dm, 1H), 6.54 (t, 1H), 6.27 (br s, 1H), 4.27 (d, 2H), 3.66 (t, 2H), 2.85 (t, 2H), 2.36/2.14 (m+m, 4H), 2.15/0.94 (m+m, 4H). HRMS calculated for C₃₀H₃₀NO₃Cl: 487.1914; found: 488.1981 (M+H).

### Example 410 (1s,4s)-4-(3-chloroanilino)-2′-{[2-(2-fluorophenyl)ethoxy]methyl}spiro[cyclohexane-1,1′-indene]-4-carboxylic acid

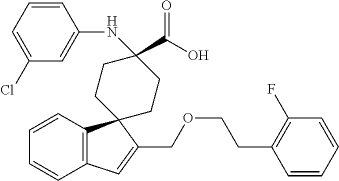 [SMILES: O=C(O)[C@]1(Nc2cccc(Cl)c2)CC[C@]2(CC1)C(COCCc1ccccc1F)=Cc1ccccc12]

Using General procedure 20 and 2-(2-fluorophenyl)ethanol as the appropriate alcohol Example 410 was obtained. HRMS calculated for C₃₀H₂₉NO₃FCl: 505.1820; found: 506.1888 (M+H).

### Example 411 (1s,4s)-4-(3-chloroanilino)-2′-{[2-(3-methylphenyl)ethoxy]methyl}spiro[cyclohexane-1,1′-indene]-4-carboxylic acid

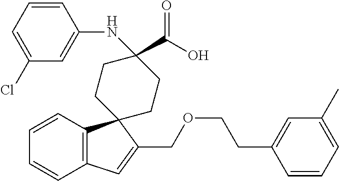 [SMILES: Cc1cccc(CCOCC2=Cc3ccccc3[C@]23CC[C@@](Nc2cccc(Cl)c2)(C(=O)O)CC3)c1]

Using General procedure 20 and 2-(m-tolyl)ethanol as the appropriate alcohol Example 411 was obtained. ¹H NMR (500 MHz, DMSO-d₆) δ ppm: 12.81 (br s, 1H), 7.65 (dm, 1H), 7.33-7.11 (m, 3H), 7.16-6.95 (m, 4H), 7.07 (t, 1H), 6.64 (t, 1H), 6.56 (m, 1H), 6.56 (m, 1H), 6.55 (t, 1H), 6.26 (br s, 1H), 4.27 (d, 2H), 3.64 (t, 2H), 2.81 (t, 2H), 2.38/2.15 (m+m, 4H), 2.24 (s, 3H), 2.16/0.96 (m+m, 4H). HRMS calculated for C₃₁H₃₂NO₃Cl: 501.2071; found: 502.2158 (M+H).

### Example 412 (1s,4s)-4-(3-chloroanilino)-2′-{[2-(3-fluorophenyl)ethoxy]methyl}spiro[cyclohexane-1,1′-indene]-4-carboxylic acid

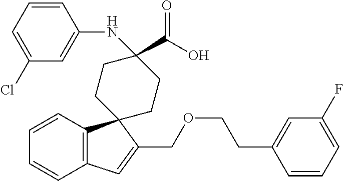 [SMILES: O=C(O)[C@]1(Nc2cccc(Cl)c2)CC[C@]2(CC1)C(COCCc1cccc(F)c1)=Cc1ccccc12]

Using General procedure 20 and 2-(3-fluorophenyl)ethanol as the appropriate alcohol Example 412 was obtained. ¹H NMR (500 MHz, DMSO-d₆) δ ppm: 12.59 (br s, 1H), 7.65 (dm, 1H), 7.33-7.11 (m, 3H), 7.26 (m, 1H), 7.08 (m, 1H), 7.07 (t, 1H), 7.07 (m, 1H), 6.99 (m, 1H), 6.64 (t, 1H), 6.55 (m, 1H), 6.55 (m, 1H), 6.54 (t, 1H), 6.26 (br s, 1H), 4.27 (d, 2H), 3.67 (t, 2H), 2.87 (t, 2H), 2.37/2.13 (m+m, 4H), 2.14/0.95 (m+m, 4H). HRMS calculated for C₃₀H₂₉NO₃FCl: 505.1820; found: 506.1900 (M+H).

### Example 413 (1s,4s)-4-(3-chloroanilino)-2′-{[2-(4-fluorophenyl)ethoxy]methyl}spiro[cyclohexane-1,1′-indene]-4-carboxylic acid

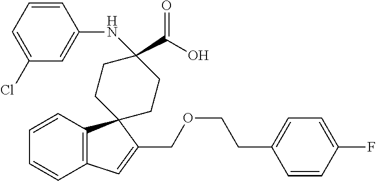 [SMILES: O=C(O)[C@]1(Nc2cccc(Cl)c2)CC[C@]2(CC1)C(COCCc1ccc(F)cc1)=Cc1ccccc12]

Using General procedure 20 and 2-(4-fluorophenyl)ethanol as the appropriate alcohol Example 413 was obtained. ¹H NMR (500 MHz, DMSO-d₆) δ ppm: 12.67 (br s, 1H), 7.65 (dm, 1H), 7.35-7.10 (m, 3H), 7.24 (m, 2H), 7.07 (t, 1H), 7.03 (m, 2H), 6.64 (t, 1H), 6.56 (m, 1H), 6.56 (m, 1H), 6.54 (t, 1H), 6.25 (br s, 1H), 4.27 (d, 2H), 3.63 (t, 2H), 2.83 (t, 2H), 2.36/2.13 (m+m, 4H), 2.14/0.93 (m+m, 4H). HRMS calculated for C₃₀H₂₉NO₃FCl: 505.1820; found: 506.1891 (M+H).

### Example 414 (1s,4s)-4-(3-chloroanilino)-2′-{[2-(2-methoxyphenyl)ethoxy]methyl}spiro[cyclohexane-1,1′-indene]-4-carboxylic acid

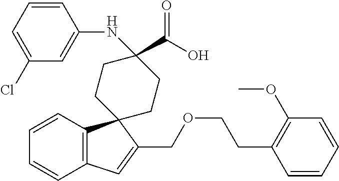 [SMILES: COc1ccccc1CCOCC1=Cc2ccccc2[C@]12CC[C@@](Nc1cccc(Cl)c1)(C(=O)O)CC2]

Using General procedure 20 and 2-(2-methoxyphenyl)ethanol as the appropriate alcohol Example 414 was obtained. HRMS calculated for C₃₁H₃₂NO₄Cl: 517.2020; found: 518.2092 (M+H).

### Example 415 (1s,4s)-4-(3-chloroanilino)-2′-{[2-(2-methylphenyl)ethoxy]methyl}spiro[cyclohexane-1,1′-indene]-4-carboxylic acid

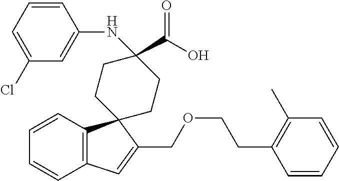 [SMILES: Cc1ccccc1CCOCC1=Cc2ccccc2[C@]12CC[C@@](Nc1cccc(Cl)c1)(C(=O)O)CC2]

Using General procedure 20 and 2-(o-tolyl)ethanol as the appropriate alcohol Example 415 was obtained. ¹H NMR (500 MHz, DMSO-d₆) δ ppm: 12.38 (br s, 1H), 7.64 (dm, 1H), 7.34-7.02 (m, 7H), 7.08 (t, 1H), 6.64 (t, 1H), 6.57 (dm, 1H), 6.56 (t, 1H), 6.55 (dm, 1H), 6.27 (br s, 1H), 4.28 (d, 2H), 3.62 (t, 2H), 2.85 (t, 2H), 2.36/2.14 (m+m, 4H), 2.24 (s, 3H), 2.16/0.94 (m+m, 4H). HRMS calculated for C₃₁H₃₂NO₃Cl: 501.2071; found: 502.2151 (M+H).

### Example 416 (1s,4s)-4-(3-chloroanilino)-2′-{[2-(4-methylphenyl)ethoxy]methyl}spiro[cyclohexane-1,1′-indene]-4-carboxylic acid

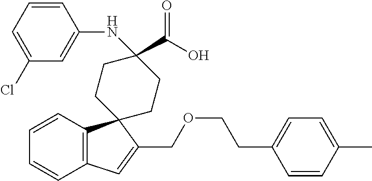 [SMILES: Cc1ccc(CCOCC2=Cc3ccccc3[C@]23CC[C@@](Nc2cccc(Cl)c2)(C(=O)O)CC3)cc1]

Using General procedure 20 and 2-(p-tolyl)ethanol as the appropriate alcohol Example 416 was obtained. ¹H NMR (500 MHz, DMSO-d₆) δ ppm: 12.76 (br s, 1H), 7.63 (dm, 1H), 7.34-6.99 (m, 7H), 7.09 (t, 1H), 6.64 (t, 1H), 6.58 (dm, 1H), 6.55 (t, 1H), 6.55 (dm, 1H), 6.28 (br s, 1H), 4.26 (d, 2H), 3.62 (t, 2H), 2.79 (t, 2H), 2.36/2.14 (m+m, 4H), 2.24 (s, 3H), 2.15/0.94 (m+m, 4H). HRMS calculated for C₃₁H₃₂NO₃Cl: 501.2071; found: 502.2135 (M+H).

### Example 417 (1s,4s)-4-(3-chloroanilino)-2′-{[2-(3-chlorophenyl)ethoxy]methyl}spiro[cyclohexane-1,1′-indene]-4-carboxylic acid

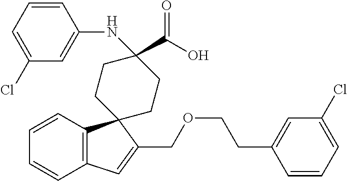 [SMILES: O=C(O)[C@]1(Nc2cccc(Cl)c2)CC[C@]2(CC1)C(COCCc1cccc(Cl)c1)=Cc1ccccc12]

Using General procedure 20 and 2-(3-chlorophenyl)ethanol as the appropriate alcohol Example 417 was obtained. ¹H NMR (500 MHz, DMSO-d₆) δ ppm: 12.82 (br s, 1H), 7.64 (dm, 1H), 7.34-7.11 (m, 7H), 7.08 (t, 1H), 6.64 (t, 1H), 6.57 (dm, 1H), 6.55 (dm, 1H), 6.54 (t, 1H), 6.28 (br s, 1H), 4.27 (d, 2H), 3.66 (t, 2H), 2.86 (t, 2H), 2.37/2.15 (m+m, 4H), 2.15/0.95 (m+m, 4H). HRMS calculated for C₃₀H₂₉NO₃Cl₂: 521.1525; found: 522.1613 (M+H).

### Example 418 (1s,4s)-4-(3-chloroanilino)-2′-{[2-(3-methoxyphenyl)ethoxy]methyl}spiro[cyclohexane-1,1′-indene]-4-carboxylic acid

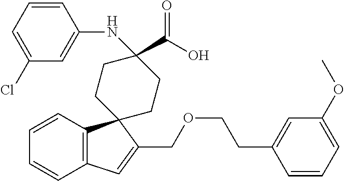 [SMILES: COc1cccc(CCOCC2=Cc3ccccc3[C@]23CC[C@@](Nc2cccc(Cl)c2)(C(=O)O)CC3)c1]

Using General procedure 20 and 2-(3-methoxyphenyl)ethanol as the appropriate alcohol Example 418 was obtained. ¹H NMR (500 MHz, DMSO-d₆) δ ppm: 12.78 (br s, 1H), 7.65 (dm, 1H), 7.33-7.12 (m, 3H), 7.15 (t, 1H), 7.07 (t, 1H), 6.83-6.73 (m, 3H), 6.64 (t, 1H), 6.56 (dm, 1H), 6.56 (t, 1H), 6.55 (dm, 1H), 6.26 (br s, 1H), 4.27 (d, 2H), 3.70 (s, 3H), 3.66 (t, 2H), 2.83 (t, 2H), 2.38/2.15 (m+m, 4H), 2.16/0.96 (m+m, 4H). HRMS calculated for C₃₁H₃₂NO₄Cl: 517.2020; found: 518.2100 (M+H).

### Example 419 (1s,4s)-4-(3-chloroanilino)-2′-{[2-(2-chlorophenyl)ethoxy]methyl}spiro[cyclohexane-1,1′-indene]-4-carboxylic acid

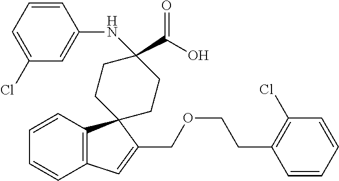 [SMILES: O=C(O)[C@]1(Nc2cccc(Cl)c2)CC[C@]2(CC1)C(COCCc1ccccc1Cl)=Cc1ccccc12]

Using General procedure 20 and 2-(2-chlorophenyl)ethanol as the appropriate alcohol Example 419 was obtained. HRMS calculated for C₃₀H₂₉NO₃Cl₂: 521.1525; found: 522.1598 (M+H).

### Example 420 (1s,4s)-4-(3-chloroanilino)-2′-{[2-(4-chlorophenyl)ethoxy]methyl}spiro[cyclohexane-1,1′-indene]-4-carboxylic acid

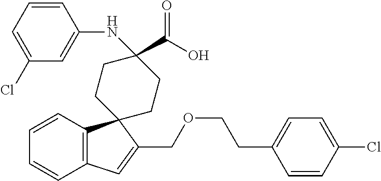 [SMILES: O=C(O)[C@]1(Nc2cccc(Cl)c2)CC[C@]2(CC1)C(COCCc1ccc(Cl)cc1)=Cc1ccccc12]

Using General procedure 20 and 2-(4-chlorophenyl)ethanol as the appropriate alcohol Example 420 was obtained. HRMS calculated for C₃₀H₂₉NO₃Cl₂: 521.1525; found: 522.1599 (M+H).

### Example 421 (1s,4s)-4-(3-chloroanilino)-2′-{[2-(4-methoxyphenyl)ethoxy]methyl}spiro[cyclohexane-1,1′-indene]-4-carboxylic acid

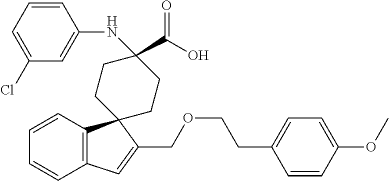 [SMILES: COc1ccc(CCOCC2=Cc3ccccc3[C@]23CC[C@@](Nc2cccc(Cl)c2)(C(=O)O)CC3)cc1]

Using General procedure 20 and 2-(4-methoxyphenyl)ethanol as the appropriate alcohol Example 421 was obtained. ¹H NMR (500 MHz, DMSO-d₆) δ ppm: 12.75 (br s, 1H), 7.65 (dm, 1H), 7.33-7.11 (m, 3H), 7.13 (m, 2H), 7.08 (m, 1H), 6.80 (m, 2H), 6.64 (t, 1H), 6.56 (dm, 1H), 6.56 (dm, 1H), 6.55 (t, 1H), 6.27 (s, 1H), 4.26 (d, 2H), 3.70 (s, 3H), 3.61 (t, 2H), 2.78 (t, 2H), 2.37/2.14 (m+m, 4H), 2.15/0.95 (m+m, 4H). HRMS calculated for C₃₁H₃₂NO₄Cl: 517.2020; found: 518.2096 (M+H).

### Example 422 (1r,4r)-4-(3-chloroanilino)-2′-(3-phenoxypropyl)spiro[cyclohexane-1,1′-indene]-4-carboxylic acid

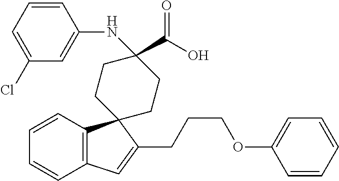 [SMILES: O=C(O)[C@]1(Nc2cccc(Cl)c2)CC[C@]2(CC1)C(CCCOc1ccccc1)=Cc1ccccc12]

Using General procedure 27a and Preparation 6a (100 mg, 0.23 mmol, 1 eq) as the appropriate 2-bromo-indene and 3-phenoxypropylzinc bromide (0.92 mL, 0.5 M in THF, 0.46 mmol, 2 eq) as the appropriate Zn reagent, the reaction was heated at 140° C. for 1 h under microwave irradiation. The reaction was allowed to cool to rt and then partitioned between DCM and water. The organic phase was washed with 1 M aq. HCl solution, brine, dried (PTFE phase separator) and concentrated in vacuo. Purification by reverse phase automated flash chromatography (CombiFlash Rf, C18 4.3 g RediSep column) eluting with a gradient of 10-100% MeCN in water afforded Example 422 as a colourless glass (32 mg, 0.07 mmol, 28%). LRMS calculated for C₃₀H₃₀ClNO₃: 487; found: 488 (M+H). ¹H NMR (400 MHz, DMSO-d₆) δ ppm: 7.67 (d, J=7.5 Hz, 1H), 7.32-7.20 (m, 4H), 7.15-7.06 (m, 2H), 6.94-6.87 (m, 3H), 6.66 (t, J=2.1 Hz, 1H), 6.60-6.55 (m, 2H), 6.55-6.52 (m, 1H), 6.37 (br s, 1H), 4.06 (t, J=6.1 Hz, 2H), 2.46-2.35 (m, 4H), 2.23-2.04 (m, 6H), 0.93-0.84 (m, 2H).

### Example 423 (1r,4r)-4-(3-chloroanilino)-2′-(3-phenoxypropyl)-2′,3′-dihydrospiro[cyclohexane-1,1′-indene]-4-carboxylic acid, enantiomer 1

and

### Example 424 (1r,4r)-4-(3-chloroanilino)-2′-(3-phenoxypropyl)-2′,3′-dihydrospiro[cyclohexane-1,1′-indene]-4-carboxylic acid, enantiomer 2

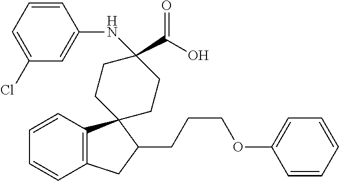

To a solution of Example 422 (110 mg, 0.23 mmol, 1 eq) in EtOH (4 mL) and THE (4 mL) was added 5% Pt/C (44 mg, 0.01 mmol, 0.05 eq) under a N₂ atmosphere. The mixture was evacuated and backfilled with N₂ (×3), then evacuated and backfilled with H₂ and shaken for 18 h at rt under an atmosphere of H₂. The reaction was filtered through celite and concentrated in vacuo. Purification by reverse phase automated flash chromatography (CombiFlash Rf, C18 13 g RediSep column) eluting with a gradient of 15-100% MeCN in water afforded a racemate. The enantiomers were separated by chiral chromatography. Column: AD, 50×500 mm, 20 μm, Eluents: EtOH+0.05% HCOOH. The enantiomer eluting earlier was collected as Example 423. ¹H NMR (500 MHz, DMSO-d₆) δ ppm: 12.67 (br s, 1H), 7.37 (dm, 1H), 7.26 (m, 2H), 7.23-7.10 (m, 3H), 7.05 (t, 1H), 6.92 (m, 2H), 6.91 (m, 1H), 6.62 (t, 1H), 6.54 (dm, 1H), 6.54 (dm, 1H), 6.21 (br s, 1H), 3.99 (t, 2H), 3.01/2.57 (dd+dd, 2H), 2.48-1.18 (m, 13H). HRMS calculated for C₃₀H₃₂NO₃Cl: 489.2071; found: 490.2148 (M+H).

The enantiomer eluting later was collected as Example 424. HRMS calculated for C₃₀H₃₂NO₃Cl: 489.2071; found: 490.2156 (M+H).

### Example 425

### Example 425A methyl (1r,4r)-4-(3-chloroanilino)-2′-[3-(4-fluorophenoxy)propyl]-2′,3′-dihydrospiro[cyclohexane-1,1′-indene]-4-carboxylate

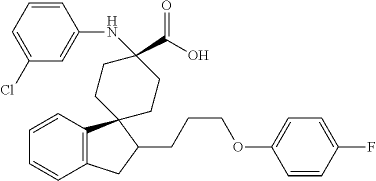

Using General procedure 40 and Preparation 9c (116 mg, 0.23 mmol, 1 eq) as the appropriate mesylate and p-fluorophenol (36 mg, 0.32 mmol, 1.4 eq) as the appropriate alcohol, racemate Example 425A was obtained as a colorless glass (104 mg, 0.2 mmol, 87%). LRMS calculated for C₃₁H₃₃ClFNO₃: 521; found: 522 (M+H). ¹H NMR (400 MHz, DMSO-d₆) δ ppm: 7.42-7.35 (m, 1H), 7.25-7.04 (m, 6H), 6.97-6.91 (m, 2H), 6.62-6.54 (m, 2H), 6.48-6.43 (m, 1H), 6.32 (s, 1H), 3.98 (t, J=6.1 Hz, 2H), 3.66 (s, 3H), 3.01 (dd, J=15.6, 7.2 Hz, 1H), 2.57 (dd, J=15.8, 7.8 Hz, 1H), 2.49-2.39 (m, 1H), 2.19-2.08 (m, 1H), 2.08-1.61 (m, 8H), 1.51-1.21 (m, 3H).

### Example 425 (1r,4r)-4-(3-chloroanilino)-2′-[3-(4-fluorophenoxy)propyl]-2′,3′-dihydrospiro[cyclohexane-1,1′-indene]-4-carboxylic acid

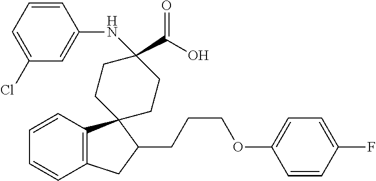

To a solution of Example 425A (104 mg, 0.2 mmol, 1 eq) in THE (4 mL) and water (1 mL) was added LiOH×H₂O (42 mg, 1 mmol, 5 eq) and the mixture was heated at 70° C. for 72 h. The reaction was allowed to cool to rt and partitioned between DCM and water. The organic phase was washed with brine, dried (MgSO₄) and concentrated in vacuo. Purification by automated flash chromatography (CombiFlash Rf, 4 g RediSep™ silica cartridge) eluting with a gradient of 0-55% EtOAc in heptane afforded racemate Example 425 as a white foam (13.3 mg, 0.03 mmol, 13%). LRMS calculated for C₃₀H₃₁ClFNO₃: 507; found: 508 (M+H). ¹H NMR (400 MHz, DMSO-d₆) δ ppm: 12.68 (br s, 1H), 7.41-7.34 (m, 1H), 7.24-7.03 (m, 6H), 6.98-6.90 (m, 2H), 6.63 (t, J=2.1 Hz, 1H), 6.58-6.52 (m, 2H), 6.22 (br s, 1H), 3.97 (t, J=6.2 Hz, 2H), 3.01 (dd, J=15.7, 7.2 Hz, 1H), 2.57 (dd, J=15.7, 7.8 Hz, 1H), 2.47-2.37 (m, 1H), 2.18-1.62 (m, 9H), 1.51-1.21 (m, 3H).

### Example 426 (1r,4r)-4-(3-chloroanilino)-2′-{3-[(pyridin-4-yl)oxy]propyl}-2′,3′-dihydrospiro[cyclohexane-1,1′-indene]-4-carboxylic acid

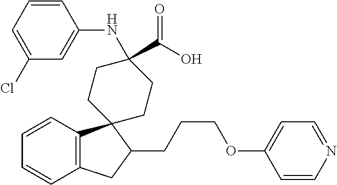

Using General procedure 30b and 4-hydroxypyridine as the appropriate alcohol and Preparation 9b (120 mg, 0.28 mmol, 1 eq) as the appropriate indane, a racemic intermediate was obtained, which was hydrolysed according to General procedure 33b to obtain Example 426 as a beige powder (16.9 mg, 0.03 mmol, 12%). LRMS calculated for C₂₉H₃₁ClN₂O₃: 490; found: 491 (M+H). ¹H NMR (400 MHz, DMSO-d₆) δ ppm: 12.70 (br s, 1H), 8.67 (d, J=6.3 Hz, 2H), 7.45 (d, J=6.3 Hz, 2H), 7.42-7.35 (m, 1H), 7.26-7.12 (m, 3H), 7.06 (t, J=8.0 Hz, 1H), 6.62 (t, J=2.1 Hz, 1H), 6.59-6.51 (m, 2H), 6.23 (br s, 1H), 4.39-4.28 (m, 2H), 3.03 (dd, J=15.6, 7.2 Hz, 1H), 2.59 (dd, J=15.7, 7.5 Hz, 1H), 2.48-2.37 (m, 1H), 2.19-1.61 (m, 9H), 1.53-1.22 (m, 3H).

### Example 427 (1r,4r)-4-(3-chloroanilino)-2′-{3-[(2-fluoropyridin-4-yl)oxy]propyl}-2′,3′-dihydrospiro[cyclohexane-1,1′-indene]-4-carboxylic acid

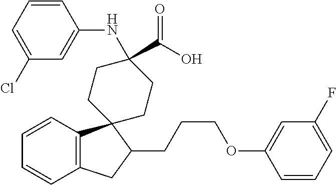

Using General procedure 40 and Preparation 9c (116 mg, 0.23 mmol, 1 eq) as the appropriate mesylate and 2-fluoro-4-hydroxypyridine (36 mg, 0.32 mmol, 1.4 eq) as the appropriate alcohol, a racemic intermediate was obtained, which was hydrolysed according to General procedure 33b to obtain Example 427 as an off-white powder (4 mg, 0.01 mmol, 3%). LRMS calculated for C₂₉H₃₀ClFN₂O₃: 508; found: 509 (M+H). ¹H NMR (400 MHz, DMSO-d₆) δ ppm: 8.02 (d, J=5.8 Hz, 1H), 7.42-7.35 (m, 1H), 7.24-7.11 (m, 3H), 7.02 (t, J=8.1 Hz, 1H), 6.94-6.90 (m, 1H), 6.75 (d, J=2.1 Hz, 1H), 6.64 (t, J=2.1 Hz, 1H), 6.58-6.52 (m, 1H), 6.52-6.47 (m, 1H), 4.14 (t, J=6.5 Hz, 2H), 3.01 (dd, J=15.7, 7.3 Hz, 1H), 2.62-2.54 (m, 1H), 2.47-2.36 (m, 1H), 2.18-1.99 (m, 2H), 1.98-1.60 (m, 7H), 1.52-1.36 (m, 2H), 1.34-1.20 (m, 1H).

### Example 428 (1r,4r)-2′-{3-[(2H-1,3-benzodioxol-5-yl)oxy]propyl}-4-(3-chloroanilino)-2′,3′-dihydrospiro[cyclohexane-1,1′-indene]-4-carboxylic acid

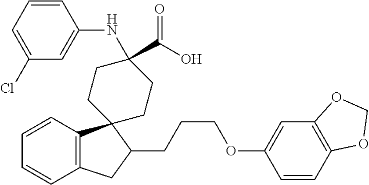

Using General procedure 40 and Preparation 9c (77 mg, 0.15 mmol, 1 eq) as the appropriate mesylate and sesamol (0.02 mL, 0.18 mmol, 1.2 eq) as the appropriate alcohol afforded an intermediate which was hydrolysed according to General procedure 33b to obtain Example 428 as a white powder (34.3 mg, 0.06 mmol, 42%). LRMS calculated for C₃₁H₃₂NOCl: 533; found: 534 (M+H). ¹H NMR (400 MHz, DMSO-d₆) δ ppm: 12.60 (br s, 1H), 7.41-7.34 (m, 1H), 7.24-7.11 (m, 3H), 7.06 (t, J=8.1 Hz, 1H), 6.78 (d, J=8.5 Hz, 1H), 6.64-6.61 (m, 2H), 6.57-6.53 (m, 2H), 6.36 (dd, J=8.5, 2.5 Hz, 1H), 6.23 (br s, 1H), 5.95 (s, 2H), 3.92 (t, J=6.0 Hz, 2H), 3.00 (dd, J=15.7, 7.2 Hz, 1H), 2.57 (dd, J=15.6, 7.7 Hz, 1H), 2.48-2.37 (m, 1H), 2.17-1.60 (m, 9H), 1.52-1.33 (m, 2H), 1.32-1.19 (m, 1H).

### Example 429 (1r,4r)-2′-[3-(4-amino-2,6-dichlorophenoxy)propyl]-4-(3-chloroanilino)-2′,3′-dihydrospiro[cyclohexane-1,1′-indene]-4-carboxylic acid

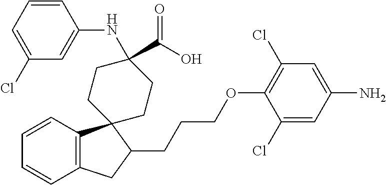

Using General procedure 40 and Preparation 9c (77 mg, 0.15 mmol, 1 eq) as the appropriate mesylate and 4-amino-2,6-dichlorophenol (33 mg, 0.18 mmol, 1.2 eq) as the appropriate alcohol afforded an intermediate which was hydrolysed according to General procedure 33b to obtain Example 429 as an off-white powder (28.2 mg, 0.05 mmol, 32%). LRMS calculated for C₃₀H₃₁N₂O₃Cl₃: 572; found: 573 (M+H). ¹H NMR (400 MHz, DMSO-d₆) δ ppm: 12.62 (br s, 1H), 7.42-7.35 (m, 1H), 7.25-7.11 (m, 3H), 7.06 (t, J=8.1 Hz, 1H), 6.62 (t, J=2.1 Hz, 1H), 6.58 (s, 2H), 6.57-6.51 (m, 2H), 6.23 (br s, 1H), 5.40 (s, 2H), 3.91-3.77 (m, 2H), 3.00 (dd, J=15.7, 7.2 Hz, 1H), 2.57 (dd, J=15.7, 8.0 Hz, 1H), 2.46-2.36 (m, 1H), 2.16-1.60 (m, 9H), 1.53-1.27 (m, 3H).

### Example 430 (1r,4r)-4-(3-chloroanilino)-2′-[3-(3-chloro-5-fluorophenoxy)propyl]-2′,3′-dihydrospiro[cyclohexane-1,1′-indene]-4-carboxylic acid

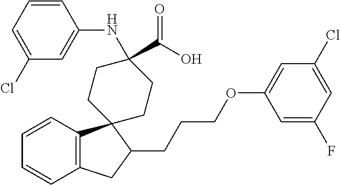

Using General procedure 40 and Preparation 9c (48 mg, 0.09 mmol, 1 eq) as the appropriate mesylate and 3-chloro-5-fluorophenol (12 μL, 0.11 mmol, 1.2 eq) as the appropriate alcohol afforded an intermediate which was hydrolysed according to General procedure 33b to obtain Example 430 as a white powder (29.8 mg, 0.05 mmol, 58%). LRMS for C₃₀H₃₀NO₃FCl₂: 541; found: 542 (M+H). ¹H NMR (400 MHz, DMSO-d₆) δ ppm: 12.71 (br s, 1H), 7.41-7.35 (m, 1H), 7.25-7.12 (m, 3H), 7.05 (t, J=8.1 Hz, 1H), 6.96 (dt, J=8.7, 2.1 Hz, 1H), 6.92-6.89 (m, 1H), 6.86 (dt, J=11.0, 2.3 Hz, 1H), 6.62 (t, J=2.1 Hz, 1H), 6.58-6.52 (m, 2H), 6.24 (br s, 1H), 4.05 (t, J=6.2 Hz, 2H), 3.01 (dd, J=15.7, 7.2 Hz, 1H), 2.57 (dd, J=15.7, 7.8 Hz, 1H), 2.47-2.37 (m, 1H), 2.18-1.60 (m, 9H), 1.52-1.21 (m, 3H).

### Example 431 (1r,4r)-4-(3-chloroanilino)-2′-[3-(2-chlorophenoxy)propyl]-2′,3′-dihydrospiro[cyclohexane-1,1′-indene]-4-carboxylic acid

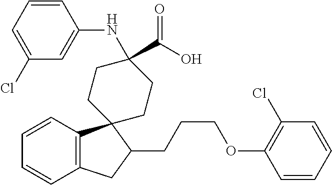

Preparation 9b (80 mg, 0.19 mmol, 1 eq) as the appropriate indane and 2-chlorophenol (29 μL, 0.28 mmol, 1.5 eq) as the appropriate alcohol were treated according to the General procedure 30b then hydrolyzed according to General procedure 33b to yield racemate Example 431 as a white powder (53.6 mg, 0.1 mmol, 55%). LRMS calculated for C₃₀H₃₁NO₃Cl₂: 523; found: 524 (M+H). ¹H NMR (400 MHz, DMSO-d₆) δ ppm: 12.70 (br s, 1H), 7.42-7.36 (m, 2H), 7.31-7.25 (m, 1H), 7.24-7.12 (m, 4H), 7.06 (t, J=8.1 Hz, 1H), 6.93 (td, J=7.6, 1.4 Hz, 1H), 6.62 (t, J=2.1 Hz, 1H), 6.57-6.52 (m, 2H), 6.21 (br s, 1H), 4.15-4.03 (m, 2H), 3.04 (dd, J=15.7, 7.2 Hz, 1H), 2.58 (dd, J=15.7, 7.7 Hz, 1H), 2.48-2.37 (m, 1H), 2.18-1.68 (m, 9H), 1.52-1.23 (m, 3H).

### Example 432

### Example 432A methyl (1r,4r)-4-(3-chloroanilino)-2′-{3-[(3-fluoropyridin-4-yl)oxy]propyl}-2′,3′-dihydrospiro[cyclohexane-1,1′-indene]-4-carboxylate

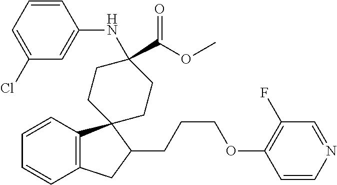

To a solution of Preparation 9c (120 mg, 0.24 mmol, 1 eq) in MeCN (3 mL) was added K₃PO₄ (66 mg, 0.47 mmol, 2 eq) and 3-fluoropyridin-4-ol (35 mg, 0.31 mmol, 1.3 eq). The mixture was heated at 75° C. for 18 h. The reaction was allowed to cool to rt and partitioned between DCM and water. The organic phase was washed with brine, dried (PTFE phase separator) and concentrated in vacuo. Purification by automated flash chromatography (CombiFlash Rf, 4 g RediSep™ silica cartridge) eluting with a gradient of 0-10% MeOH in DCM afforded racemate Example 432A as a colorless glass (50 mg, 0.1 mmol, 40%). LRMS calculated for C₃₀H₃₂ClFN₂O₃: 522; found: 523 (M+H). ¹H NMR (400 MHz, DMSO-d₆) δ ppm: 8.41 (d, J=3.4 Hz, 1H), 8.27 (d, J=5.5 Hz, 1H), 7.43-7.36 (m, 1H), 7.26 (dd, J=7.6, 5.5 Hz, 1H), 7.24-7.12 (m, 3H), 7.06 (t, J=8.0 Hz, 1H), 6.61-6.54 (m, 2H), 6.48-6.43 (m, 1H), 6.31 (s, 1H), 4.19 (t, J=6.5 Hz, 2H), 3.66 (s, 3H), 3.03 (dd, J=15.7, 7.2 Hz, 1H), 2.58 (dd, J=15.6, 7.9 Hz, 1H), 2.48-2.38 (m, 1H), 2.18-1.61 (m, 9H), 1.50-1.23 (m, 3H).

### Example 432 (1r,4r)-4-(3-chloroanilino)-2′-{3-[(3-fluoropyridin-4-yl)oxy]propyl}-2′,3′-dihydrospiro[cyclohexane-1,1′-indene]-4-carboxylic acid

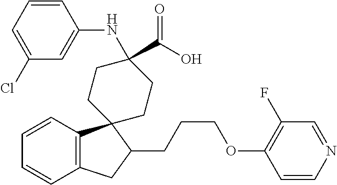

Using General procedure 33b and Example 432A (50 mg, 0.1 mmol, 1 eq) as the appropriate ester, Example 432 was obtained as a white solid (18.4 mg, 0.04 mmol, 38%). LRMS calculated for C₂₉H₃₀ClFN₂O₃: 508; found: 509 (M+H). ¹H NMR (400 MHz, DMSO-d₆) δ ppm: 12.71 (br s, 1H), 8.41 (d, J=3.4 Hz, 1H), 8.27 (d, J=5.5 Hz, 1H), 7.42-7.33 (m, 1H), 7.26 (dd, J=7.6, 5.5 Hz, 1H), 7.24-7.12 (m, 3H), 7.06 (t, J=8.1 Hz, 1H), 6.62 (t, J=2.1 Hz, 1H), 6.58-6.52 (m, 2H), 6.21 (br s, 1H), 4.19 (t, J=6.5 Hz, 2H), 3.03 (dd, J=15.6, 7.2 Hz, 1H), 2.58 (dd, J=15.7, 7.7 Hz, 1H), 2.47-2.37 (m, 1H), 2.18-1.61 (m, 9H), 1.51-1.23 (m, 3H).

### Example 433

### Example 433A tert-butyl 3-{3-[(1r,4r)-4-(3-chloroanilino)-4-(methoxycarbonyl)-2′,3′-dihydrospiro[cyclohexane-1,1′-inden]-2′-yl]propoxy}-4,5,6,7-tetrahydro-1H-indazole-1-carboxylate

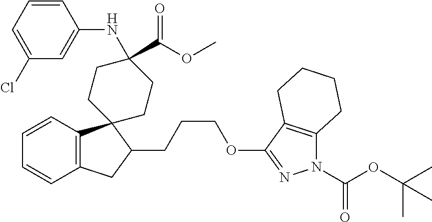

Using General procedure 40 and Preparation 9c (110 mg, 0.22 mmol, 1 eq) as the appropriate mesylate and tert-butyl 3-hydroxy-4,5,6,7-tetrahydroindazole-1-carboxylate (62 mg, 0.26 mmol, 1.2 eq) as the appropriate alcohol, racemate Example 433A was obtained as a colorless glass (86 mg, 0.13 mmol, 61%). LRMS calculated for C₃₇H₄₆ClN₃O₅: 647; found: 648 (M+H). ¹H NMR (400 MHz, DMSO-d₆) δ ppm: 7.42-7.34 (m, 1H), 7.23-7.12 (m, 3H), 7.06 (t, J=8.0 Hz, 1H), 6.61-6.54 (m, 2H), 6.48-6.43 (m, 1H), 6.32 (s, 1H), 4.26-4.14 (m, 2H), 3.66 (s, 3H), 3.02 (dd, J=15.9, 7.3 Hz, 1H), 2.79-2.71 (m, 2H), 2.56 (dd, J=15.9, 8.0 Hz, 1H), 2.48-2.37 (m, 1H), 2.25-2.08 (m, 3H), 2.08-1.96 (m, 2H), 1.96-1.53 (m, 8H), 1.53 (s, 10H), 1.49-1.23 (m, 3H).

### Example 433 (1r,4r)-4-(3-chloroanilino)-2′-{3-[(4,5,6,7-tetrahydro-1H-indazol-3-yl)oxy]propyl}-2′,3′-dihydrospiro[cyclohexane-1,1′-indene]-4-carboxylic acid

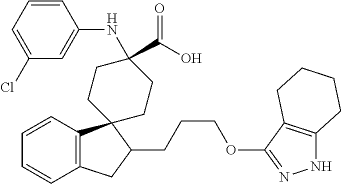

Using General procedure 33b and Example 433A (86 mg, 0.13 mmol, 1 eq) as the appropriate ester, racemate Example 433 was obtained as a white powder (26.3 mg, 0.05 mmol, 37%). LRMS calculated for C₃₁H₃₆ClN₃O₃: 533; found: 534 (M+H). ¹H NMR (400 MHz, DMSO-d₆) δ ppm: 12.68 (br s, 1H), 11.23 (br s, 1H), 7.40-7.34 (m, 1H), 7.23-7.11 (m, 3H), 7.05 (t, J=8.1 Hz, 1H), 6.62 (t, J=2.1 Hz, 1H), 6.58-6.62 (m, 2H), 6.23 (br s, 1H), 4.08 (t, J=6.3 Hz, 2H), 3.00 (dd, J=15.8, 7.2 Hz, 1H), 2.55 (dd, J=15.8, 7.8 Hz, 1H), 2.49-2.36 (m, 3H), 2.21 (t, J=6.0 Hz, 2H), 2.17-2.07 (m, 1H), 2.07-1.93 (m, 2H), 1.93-1.54 (m, 10H), 1.50-1.34 (m, 2H), 1.29-1.15 (m, 1H).

### Example 434

### Example 434A methyl (1r,4r)-4-(3-chloroanilino)-2′-{3-[(methanesulfonyl)oxy]propyl}spiro[cyclohexane-1,1′-indene]-4-carboxylate

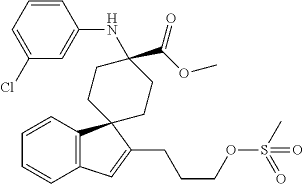 [SMILES: COC(=O)[C@]1(Nc2cccc(Cl)c2)CC[C@]2(CC1)C(CCCOS(C)(=O)=O)=Cc1ccccc12]

To a solution of Preparation 9a (78 mg, 0.18 mmol, 1 eq) in DCM (2 mL) was added TEA (51 μL, 0.37 mmol, 2 eq) followed by MsCl (17 μL, 0.22 mmol, 1.2 eq) and the mixture was stirred at rt for 2 h. The reaction was partitioned between DCM and water, and the organic phase was washed with water, brine and dried (MgSO₄). Filtration and concentration in vacuo afforded Example 434A as a colourless gum (92 mg, 0.18 mmol, 100%). LRMS calculated for C₂₆H₃₀ClNO₅S: 503; found 504 (M+H). ¹H NMR (400 MHz, DMSO-d₆) δ ppm: 7.67 (d, J=7.5 Hz, 1H), 7.31 (dd, J=7.5, 1.3 Hz, 1H), 7.24 (td, J=7.4, 0.9 Hz, 1H), 7.16-7.07 (m, 2H), 6.63 (t, J=2.1 Hz, 1H), 6.62-6.58 (m, 1H), 6.54-6.44 (m, 3H), 4.32 (t, J=6.3 Hz, 2H), 3.70 (s, 3H), 3.20 (s, 3H), 2.48-2.36 (m, 2H), 2.35-2.27 (m, 2H), 2.27-2.01 (m, 6H), 0.97-0.87 (m, 2H).

### Example 434 (1r,4r)-4-(3-chloroanilino)-2′-{3-[(1H-indol-4-yl)oxy]propyl}spiro[cyclohexane-1,1′-indene]-4-carboxylic acid

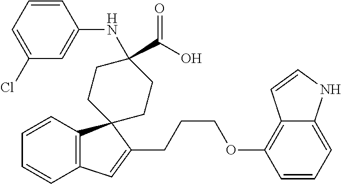 [SMILES: O=C(O)[C@]1(Nc2cccc(Cl)c2)CC[C@]2(CC1)C(CCCOc1cccc3[nH]ccc13)=Cc1ccccc12]

Using General procedure 40 and Example 434A (95 mg, 0.19 mmol, 1 eq) as the appropriate mesylate and 4-hydroxyindole (30 mg, 0.23 mmol, 1.2 eq) as the appropriate alcohol (and heating at 90° C. for 5H), an intermediate was obtained which was hydrolyzed according to General Procedure 33b to obtain Example 434 as a white foam (18 mg, 0.03 mmol, 18%). LRMS calculated for C₃₂H₃₁ClN₂O₃: 526; found: 527 (M+H). ¹H NMR (400 MHz, DMSO-d₆) δ ppm: 12.80 (br s, 1H), 11.04 (s, 1H), 7.67 (d, J=7.5 Hz, 1H), 7.30 (dd, J=7.5, 1.2 Hz, 1H), 7.23 (td, J=7.5, 0.9 Hz, 1H), 7.16-7.06 (m, 3H), 7.00-6.93 (m, 2H), 6.65 (t, J=2.1 Hz, 1H), 6.61-6.55 (m, 3H), 6.49 (dd, J=6.3, 2.2 Hz, 1H), 6.45-6.33 (m, 2H), 4.18 (t, J=6.2 Hz, 2H), 2.50-2.36 (m, 4H), 2.25-2.11 (m, 6H), 0.96-0.85 (m, 2H).

### Example 435 (1r,4r)-4-(3-chloroanilino)-2′-{3-[(1-oxo-2,3-dihydro-1H-inden-4-yl)oxy]propyl}-2′,3′-dihydrospiro[cyclohexane-1,1′-indene]-4-carboxylic acid

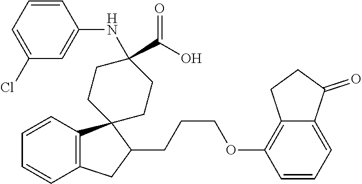

Using General procedure 40 and Preparation 9c (77 mg, 0.15 mmol, 1 eq) as the appropriate mesylate and 4-hydroxy-2,3-dihydroinden-1-one (27 mg, 0.18 mmol, 1.2 eq) as the appropriate alcohol afforded an intermediate which was hydrolyzed according to General procedure 33b to obtain Example 435 as a white powder (8.6 mg, 0.02 mmol, 10%). LRMS calculated for C₃₃H₃₄ClNO₄: 543; found: 544 (M+H). ¹H NMR (400 MHz, DMSO-d₆) δ ppm: 12.71 (br s, 1H), 7.42-7.34 (m, 2H), 7.27-7.11 (m, 5H), 7.05 (t, J=8.1 Hz, 1H), 6.62 (t, J=2.1 Hz, 1H), 6.57-6.50 (m, 2H), 6.24 (br s, 1H), 4.20-4.07 (m, 2H), 3.04 (dd, J=15.7, 7.2 Hz, 1H), 2.92 (t, J=5.6 Hz, 2H), 2.63-2.54 (m, 3H), 2.48-2.38 (m, 1H), 2.18-1.84 (m, 6H), 1.84-1.70 (m, 3H), 1.52-1.24 (m, 3H).

### Example 436 (1r,4r)-4-(3-chloroanilino)-2′-[3-(4-chloro-3,5-dimethylphenoxy)propyl]-2′,3′-dihydrospiro[cyclohexane-1,1′-indene]-4-carboxylic acid

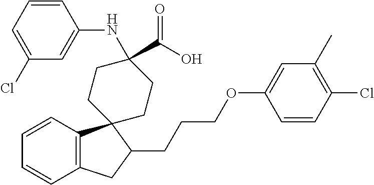

Using General procedure 40 and Preparation 9c (77 mg, 0.15 mmol, 1 eq) as the appropriate mesylate and 4-chloro-3,5-dimethylphenol (29 mg, 0.18 mmol, 1.2 eq) as the appropriate alcohol afforded an intermediate which was hydrolyzed according to General procedure 33b to obtain Example 436 as a white powder (36 mg, 0.07 mmol, 43%). LRMS calculated for C₃₂H₃₅Cl₂NO₃: 551; found: 552 (M+H). ¹H NMR (400 MHz, DMSO-d₆) δ ppm: 12.61 (br s, 1H), 7.41-7.35 (m, 1H), 7.24-7.12 (m, 3H), 7.05 (t, J=8.1 Hz, 1H), 6.79 (s, 2H), 6.62 (t, J=2.1 Hz, 1H), 6.57-6.52 (m, 2H), 6.23 (br s, 1H), 3.97 (t, J=6.2 Hz, 2H), 3.01 (dd, J=15.7, 7.2 Hz, 1H), 2.57 (dd, J=15.7, 7.7 Hz, 1H), 2.47-2.37 (m, 1H), 2.28 (s, 6H), 2.18-1.60 (m, 9H), 1.52-1.35 (m, 2H), 1.34-1.21 (m, 1H).

### Example 437 (1r,4r)-4-(3-chloroanilino)-2′-{3-[(thieno[3,2-d]pyrimidin-4-yl)oxy]propyl}-2′,3′-dihydrospiro[cyclohexane-1,1′-indene]-4-carboxylic acid

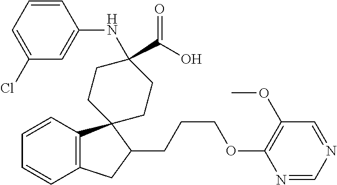

Using General procedure 31b and Preparation 9b (107 mg, 0.25 mmol, 1 eq) as the appropriate alcohol and 4-chlorothieno[3,2-d]pyrimidine (64 mg, 0.38 mmol, 1.5 eq) as the appropriate aryl chloride, racemate Example 437 was obtained as a white powder (21.2 mg, 0.04 mmol, 16%). LRMS calculated for C₃₀H₃₀ClN₃O₃S: 547; found: 548 (M+H). ¹H NMR (400 MHz, DMSO-d₆) δ ppm: 8.75 (s, 1H), 8.30 (d, J=5.4 Hz, 1H), 7.59 (d, J=5.4 Hz, 1H), 7.42-7.34 (m, 1H), 7.24-7.11 (m, 3H), 7.04 (t, J=8.1 Hz, 1H), 6.61 (t, J=2.1 Hz, 1H), 6.57-6.51 (m, 2H), 6.22 (br s, 1H), 4.60 (t, J=6.6 Hz, 2H), 3.03 (dd, J=15.7, 7.2 Hz, 1H), 2.58 (dd, J=15.7, 7.8 Hz, 1H), 2.47-2.35 (m, 1H), 2.17-1.65 (m, 9H), 1.51-1.21 (m, 3H).

### Example 438 (1r,4r)-4-(3-chloroanilino)-2′-{3-[(5-methoxypyrimidin-4-yl)oxy]propyl}-2′,3′-dihydrospiro[cyclohexane-1,1′-indene]-4-carboxylic acid

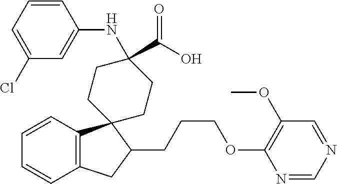

Using General procedure 31b and Preparation 9b (107 mg, 0.25 mmol, 1 eq) as the appropriate alcohol and 4-chloro-5-methoxypyrimidine (54 mg, 0.38 mmol, 1.5 eq) as the appropriate aryl chloride, racemate Example 438 was obtained as a white powder (17.9 mg, 0.03 mmol, 14%). LRMS calculated for C₂₉H₃₂ClN₃O₄: 521; found: 522 (M+H). ¹H NMR (400 MHz, DMSO-d₆) δ ppm: 12.72 (br s, 1H), 8.35 (s, 1H), 8.22 (s, 1H), 7.41-7.34 (m, 1H), 7.24-7.11 (m, 3H), 7.05 (t, J=8.1 Hz, 1H), 6.62 (t, J=2.1 Hz, 1H), 6.57-6.51 (m, 2H), 6.22 (br s, 1H), 4.45-4.34 (m, 2H), 3.82 (s, 3H), 3.01 (dd, J=15.7, 7.2 Hz, 1H), 2.56 (dd, J=15.7, 7.6 Hz, 1H), 2.47-2.36 (m, 1H), 2.18-1.81 (m, 6H), 1.81-1.57 (m, 3H), 1.51-1.34 (m, 2H), 1.33-1.19 (m, 1H).

### Example 439

### Example 439A methyl (1r,2′R,4R)-4-(3-chloroanilino)-2′-{3-[(thieno[3,2-b]pyridin-7-yl)oxy]propyl}-2′,3′-dihydrospiro[cyclohexane-1,1′-indene]-4-carboxylate

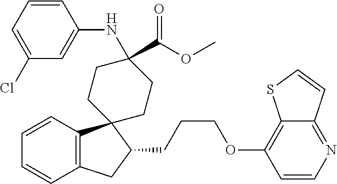 [SMILES: COC(=O)[C@]1(Nc2cccc(Cl)c2)CC[C@@]2(CC1)c1ccccc1C[C@H]2CCCOc1ccnc2ccsc12]

To a solution of Preparation 9b1 (50 mg, 0.12 mmol, 1 eq) in THE (2 mL), cooled to 0° C., was added thieno[3,2-b]pyridine-7-ol (27 mg, 0.18 mmol, 1.5 eq) and PPh₃ (46 mg, 0.18 mmol, 1.5 eq) followed by DIAD (35 μL, 0.18 mmol, 1.5 eq) dropwise and the mixture was stirred at rt for 18 h. The reaction was partitioned between DCM and water, and the organic phase was washed with brine, dried (MgSO₄) and concentrated in vacuo. Purification by automated flash column chromatography (CombiFlash Rf, 12 g RediSep™ silica cartridge) eluting with a gradient of 0-80% EtOAc in heptane afforded Example 439A as a white solid (63.9 mg, 0.11 mmol, 98%). LRMS calculated for C₃₂H₃₃ClN₂O₃S: 560; found: 561 (M+H). ¹H NMR (400 MHz, DMSO-d₆) δ ppm: 8.52 (d, J=5.4 Hz, 1H), 7.98 (d, J=5.4 Hz, 1H), 7.50 (d, J=5.4 Hz, 1H), 7.42-7.36 (m, 1H), 7.25-7.13 (m, 3H), 7.06 (t, J=8.1 Hz, 1H), 7.01 (d, J=5.4 Hz, 1H), 6.61-6.55 (m, 2H), 6.48-6.43 (m, 1H), 6.31 (s, 1H), 4.38-4.27 (m, 2H), 3.66 (s, 3H), 3.06 (dd, J=15.8, 7.2 Hz, 1H), 2.60 (dd, J=15.8, 7.8 Hz, 1H), 2.49-2.39 (m, 1H), 2.19-1.69 (m, 9H), 1.50-1.24 (m, 3H).

### Example 439 (1r,2′R,4R)-4-(3-chloroanilino)-2′-{3-[(thieno[3,2-b]pyridin-7-yl)oxy]propyl}-2′,3′-dihydrospiro[cyclohexane-1,1′-indene]-4-carboxylic acid

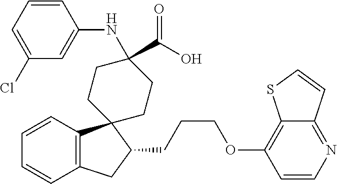 [SMILES: O=C(O)[C@]1(Nc2cccc(Cl)c2)CC[C@@]2(CC1)c1ccccc1C[C@H]2CCCOc1ccnc2ccsc12]

To a solution of Example 439A (63.9 mg, 0.11 mmol, 1 eq) in MeOH (4 mL) was added a solution of LiOH×H₂O (38 mg, 0.91 mmol, 8 eq) in water (0.4 mL) and the mixture was heated at 120° C. for 1 h under microwave irradiation. The reaction was allowed to cool to rt and partitioned between DCM and water. The organic phase was washed with 0.5 M aq. HCl solution, brine, dried (MgSO₄) and concentrated in vacuo. Purification by automated flash chromatography (CombiFlash Rf, 4 g RediSep™ silica cartridge) eluting with a gradient of 0-8% MeOH in DCM afforded Example 439 as a white solid (24 mg, 0.04 mmol, 39%). LRMS calculated for C₃1H₃1ClN₂O₃S: 546; found: 547 (M+H). ¹H NMR (400 MHz, DMSO-d₆) δ ppm: 12.72 (br s, 1H), 8.52 (d, J=5.4 Hz, 1H), 7.98 (d, J=5.4 Hz, 1H), 7.50 (d, J=5.4 Hz, 1H), 7.41-7.34 (m, 1H), 7.25-7.12 (m, 3H), 7.05 (t, J=8.1 Hz, 1H), 7.01 (d, J=5.4 Hz, 1H), 6.62 (t, J=2.1 Hz, 1H), 6.57-6.52 (m, 2H), 6.20 (br s, 1H), 4.38-4.27 (m, 2H), 3.05 (dd, J=15.7, 7.2 Hz, 1H), 2.60 (dd, J=15.7, 7.7 Hz, 1H), 2.47-2.37 (m, 1H), 2.19-1.69 (m, 9H), 1.51-1.26 (m, 3H).

### Example 440

### Example 440A methyl (1r,2′S,4S)-4-(3-chloroanilino)-2′-{3-[(thieno[3,2-b]pyridin-7-yl)oxy]propyl}-2′,3′-dihydrospiro[cyclohexane-1,1′-indene]-4-carboxylate

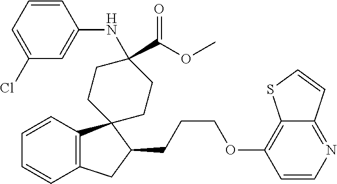 [SMILES: COC(=O)[C@]1(Nc2cccc(Cl)c2)CC[C@@]2(CC1)c1ccccc1C[C@@H]2CCCOc1ccnc2ccsc12]

To a solution of Preparation 9b2 (50 mg, 0.12 mmol, 1 eq) in THE (2 mL), cooled to 0° C., was added thieno[3,2-b]pyridine-7-ol (27 mg, 0.18 mmol, 1.5 eq) and PPh₃ (46 mg, 0.18 mmol, 1.5 eq) followed by DIAD (35 μL, 0.18 mmol, 1.5 eq) dropwise and the mixture was stirred at rt for 18 h. The reaction was partitioned between DCM and water, and the organic phase was washed with brine, dried (MgSO₄) and concentrated in vacuo. Purification by automated flash column chromatography (CombiFlash Rf, 12 g RediSep™ silica cartridge) eluting with a gradient of 0-60% EtOAc in heptane afforded Example 440A as a white solid (55.6 mg, 0.10 mmol, 85%). LRMS calculated for C₃₂H₃₃ClN₂O₃S: 560; found: 561 (M+H). ¹H NMR (400 MHz, DMSO-d₆) δ ppm: 8.52 (d, J=5.4 Hz, 1H), 7.98 (d, J=5.4 Hz, 1H), 7.51 (d, J=5.4 Hz, 1H), 7.42-7.36 (m, 1H), 7.25-7.12 (m, 3H), 7.06 (t, J=8.1 Hz, 1H), 7.01 (d, J=5.4 Hz, 1H), 6.61-6.56 (m, 2H), 6.48-6.43 (m, 1H), 6.31 (s, 1H), 4.38-4.27 (m, 2H), 3.66 (s, 3H), 3.06 (dd, J=15.6, 7.2 Hz, 1H), 2.60 (dd, J=15.6, 7.8 Hz, 1H), 2.48-2.39 (m, 1H), 2.20-1.69 (m, 9H), 1.50-1.24 (m, 3H).

### Example 440 (1r,2′S,4S)-4-(3-chloroanilino)-2′-{3-[(thieno[3,2-b]pyridin-7-yl)oxy]propyl}-2′,3′-dihydrospiro[cyclohexane-1,1′-indene]-4-carboxylic acid

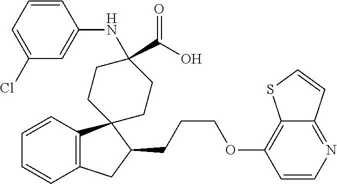 [SMILES: O=C(O)[C@]1(Nc2cccc(Cl)c2)CC[C@@]2(CC1)c1ccccc1C[C@@H]2CCCOc1ccnc2ccsc12]

To a solution of Example 440A (55.6 mg, 0.1 mmol, 1 eq) in MeOH (4 mL) was added a solution of LiOH×H₂O (33 mg, 0.79 mmol, 8 eq) in water (0.4 mL) and the mixture was heated at 100° C. for 80 min under microwave irradiation. The reaction was allowed to cool to rt, concentrated and the aq. acidified to pH 3. The mixture was extracted with DCM and the organic phase was washed brine, dried (MgSO₄) and concentrated in vacuo to afford Example 440 as a white solid (29.4 mg, 0.05 mmol, 54%). LRMS calculated for C₃₁H₃₁ClN₂O₃S: 546; found: 547 (M+H). ¹H NMR (400 MHz, DMSO-d₆) δ ppm: 12.70 (br s, 1H), 8.62 (d, J=5.8 Hz, 1H), 8.10 (d, J=5.4 Hz, 1H), 7.56 (d, J=5.4 Hz, 1H), 7.41-7.34 (m, 1H), 7.25-7.12 (m, 4H), 7.05 (t, J=8.1 Hz, 1H), 6.62 (t, J=2.1 Hz, 1H), 6.57-6.51 (m, 2H), 6.21 (br s, 1H), 4.44-4.33 (m, 2H), 3.06 (dd, J=15.7, 7.2 Hz, 1H), 2.60 (dd, J=15.6, 7.7 Hz, 1H), 2.47-2.37 (m, 1H), 2.20-1.69 (m, 9H), 1.51-1.27 (m, 3H).

### Example 441 (1r,4r)-4-(3-chloroanilino)-2′-{3-[(5-methylpyrimidin-4-yl)oxy]propyl}-2′,3′-dihydrospiro[cyclohexane-1,1′-indene]-4-carboxylic acid

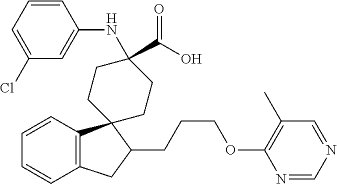

Using General procedure 31b and Preparation 9b (100 mg, 0.23 mmol, 1 eq) as the appropriate alcohol and 4-chloro-5-methylpyridine (45 mg, 0.35 mmol, 1.5 eq) as the appropriate aryl chloride, racemate Example 441 was obtained as a yellow powder (36.9 mg, 0.07 mmol, 31%). LRMS calculated for C₂₉H₃₂ClN₃O₃: 505; found: 506 (M+H). ¹H NMR (400 MHz, DMSO-d₆) δ ppm: 12.69 (br s, 1H), 8.60 (s, 1H), 8.34 (s, 1H), 7.42-7.34 (m, 1H), 7.23-7.11 (m, 3H), 7.06 (t, J=8.1 Hz, 1H), 6.61 (t, J=2.1 Hz, 1H), 6.58-6.50 (m, 2H), 6.22 (br s, 1H), 4.46-4.35 (m, 2H), 3.01 (dd, J=15.7, 7.3 Hz, 1H), 2.56 (dd, J=15.9, 8.3 Hz, 1H), 2.47-2.36 (m, 1H), 2.16-1.63 (m, 12H), 1.51-1.22 (m, 3H).

### Example 442 (1r,4r)-4-(3-chloroanilino)-2′-{3-[(thieno[3,2-b]pyridin-7-yl)oxy]propyl}spiro[cyclohexane-1,1′-indene]-4-carboxylic acid

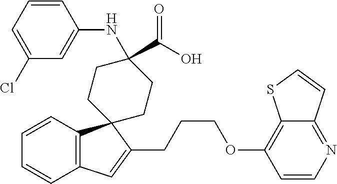 [SMILES: O=C(O)[C@]1(Nc2cccc(Cl)c2)CC[C@]2(CC1)C(CCCOc1ccnc3ccsc13)=Cc1ccccc12]

Using General procedure 30b and thieno[3,2-b]pyridin-7-ol (53 mg, 0.35 mmol, 1.5 eq) as the appropriate alcohol and Preparation 9a (100 mg, 0.23 mmol, 1 eq) as the appropriate indene afforded an intermediate which was hydrolyzed according to General procedure 33b to yield Example 442 as a white powder (36.7 mg, 0.07 mmol, 29%). LRMS calculated for C₃₁H₂₉ClN₂O₃S: 544; found: 545 (M+H). ¹H NMR (400 MHz, DMSO-d₆) δ ppm: 8.52 (d, J=5.4 Hz, 1H), 7.93 (d, J=5.4 Hz, 1H), 7.68 (d, J=7.6 Hz, 1H), 7.50 (d, J=5.4 Hz, 1H), 7.30 (dd, J=7.5, 1.3 Hz, 1H), 7.23 (t, J=7.4 Hz, 1H), 7.15-7.05 (m, 2H), 7.02 (d, J=5.4 Hz, 1H), 6.64 (t, J=2.1 Hz, 1H), 6.60-6.54 (m, 3H), 6.35 (br s, 1H), 4.39 (t, J=6.3 Hz, 2H), 2.48-2.35 (m, 4H), 2.26-2.11 (m, 6H), 0.94-0.85 (m, 2H).

### Example 443 (1r,4r)-4-(3-chloroanilino)-2′-{3-[2-(trifluoromethyl)phenoxy]propyl}-2′,3′-dihydrospiro[cyclohexane-1,1′-indene]-4-carboxylic acid

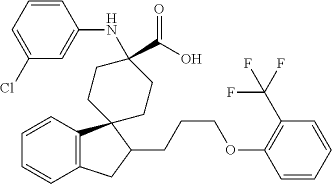

Using General procedure 30b and o-trifluoromethylphenol (46 mg, 0.28 mmol, 1.5 eq) as the appropriate alcohol and Preparation 9b (80 mg, 0.19 mmol, 1 eq) as the appropriate indane afforded an intermediate which was hydrolyzed according to General procedure 33b to yield racemate Example 443 as a white foam (57.7 mg, 0.10 mmol, 55%). LRMS calculated for C₃₁H₃₁ClF₃NO₃: 557; found: 558 (M+H). ¹H NMR (400 MHz, DMSO-d₆) δ ppm: 12.69 (br s, 1H), 7.64-7.56 (m, 2H), 7.41-7.34 (m, 1H), 7.28-7.12 (m, 4H), 7.10-7.02 (m, 2H), 6.62 (t, J=2.1 Hz, 1H), 6.57-6.51 (m, 2H), 6.19 (br s, 1H), 4.19-4.08 (m, 2H), 3.03 (dd, J=15.7, 7.2 Hz, 1H), 2.57 (dd, J=15.6, 7.6 Hz, 1H), 2.48-2.38 (m, 1H), 2.18-1.95 (m, 3H), 1.94-1.65 (m, 6H), 1.51-1.22 (m, 3H).

### Example 444

### Example 444A methyl (1r,4r)-4-(3-chloroanilino)-2′-{3-[(quinolin-4-yl)oxy]propyl}-2′,3′-dihydrospiro[cyclohexane-1,1′-indene]-4-carboxylate

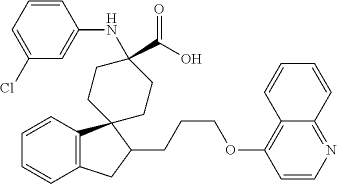

Using General procedure 31a and Preparation 9b (60 mg, 0.14 mmol, 1 eq) as the appropriate alcohol and 4-chloroquinoline (28 mg, 0.17 mmol, 1.2 eq) as the appropriate aryl chloride, racemate Example 444A was obtained as a yellow glass (62 mg, 0.11 mmol, 80%). LRMS calculated for C₃₄H₃₅ClN₂O₃: 554; found: 555 (M+H). ¹H NMR (400 MHz, DMSO-d₆) δ ppm: 8.72 (d, J=5.2 Hz, 1H), 8.14 (dd, J=8.3, 1.4 Hz, 1H), 7.96-7.90 (m, 1H), 7.76-7.69 (m, 1H), 7.53-7.48 (m, 1H), 7.43-7.36 (m, 1H), 7.27-7.21 (m, 1H), 7.21-7.13 (m, 2H), 7.05-6.99 (m, 2H), 6.60-6.53 (m, 2H), 6.45 (ddd, J=8.3, 2.2, 0.9 Hz, 1H), 6.32 (s, 1H), 4.35-4.24 (m, 2H), 3.66 (s, 3H), 3.08 (dd, J=15.7, 7.3 Hz, 1H), 2.64 (dd, J=15.7, 7.8 Hz, 1H), 2.49-2.39 (m, 1H), 2.20-1.74 (m, 9H), 1.53-1.32 (m, 3H).

### Example 444 (1r,4r)-4-(3-chloroanilino)-2′-{3-[(quinolin-4-yl)oxy]propyl}-2′,3′-dihydrospiro[cyclohexane-1,1′-indene]-4-carboxylic acid

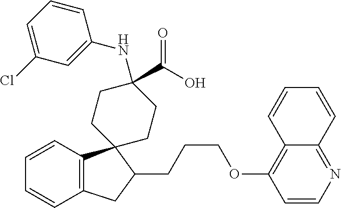

To a solution of Example 444A (60 mg, 0.11 mmol, 1 eq) in MeOH (2 mL) and THE (1 mL) was added LiOH×H₂O (32 mg, 0.76 mmol, 7 eq) and the mixture was heated at 110° C. for 40 min under microwave irradiation. The reaction was allowed to cool to rt and partitioned between DCM and water. The organic phase was washed with 1 N aq. HCl solution, brine, dried (phase separator) and concentrated in vacuo. Purification by reverse phase automated flash chromatography (CombiFlash Rf, C18 4.3 g RediSep column) eluting with a gradient of 25-85% MeCN in water afforded racemate Example 444 as a white powder (13.5 mg, 0.02 mmol, 23%). LRMS calculated for C₃₃H₃₃ClN₂O₃: 540; found: 541 (M+H). ¹H NMR (400 MHz, DMSO-d₆) δ ppm: 8.71 (d, J=5.2 Hz, 1H), 8.14 (dd, J=8.4, 1.5 Hz, 1H), 7.94 (d, J=8.5 Hz, 1H), 7.75-7.70 (m, 1H), 7.54-7.47 (m, 1H), 7.43-7.36 (m, 1H), 7.26-7.20 (m, 1H), 7.20-7.12 (m, 2H), 7.04-6.97 (m, 2H), 6.61 (t, J=2.1 Hz, 1H), 6.56-6.51 (m, 2H), 6.23 (br s, 1H), 4.36-4.23 (m, 2H), 3.07 (dd, J=15.7, 7.2 Hz, 1H), 2.63 (dd, J=15.7, 7.8 Hz, 1H), 2.49-2.37 (m, 1H), 2.20-1.72 (m, 9H), 1.54-1.32 (m, 3H).

### Example 445 (1r,4r)-4-(3-chloroanilino)-2′-{3-[(1H-pyrrolo[2,3-b]pyridin-4-yl)oxy]propyl}-2′,3′-dihydrospiro[cyclohexane-1,1′-indene]-4-carboxylic acid

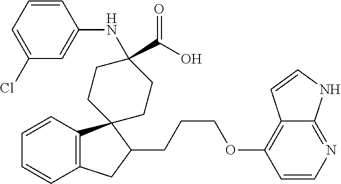

Using General procedure 31a and Preparation 9b (90 mg, 0.21 mmol, 1 eq) as the appropriate alcohol and 4-chloro-1-[tri(propan-2-yl)silyl]-1H-pyrrolo[2,3-b]pyridine (78 mg, 0.25 mmol, 1.2 eq) as the appropriate aryl chloride afforded an intermediate which was hydrolyzed according to General procedure 33b to yield racemate Example 445 as a white powder (8.6 mg, 0.02 mmol, 8%). LRMS calculated for C₃₁H₃₂ClN₃O₃: 529; found: 530 (M+H). ¹H NMR (400 MHz, DMSO-d₆) δ ppm: 11.52 (s, 1H), 8.05 (d, J=5.5 Hz, 1H), 7.42-7.34 (m, 1H), 7.25-7.11 (m, 4H), 7.04 (t, J=8.0 Hz, 1H), 6.65-6.60 (m, 2H), 6.58-6.51 (m, 2H), 6.40 (dd, J=3.4, 1.9 Hz, 1H), 6.22 (br s, 1H), 4.28-4.15 (m, 2H), 3.05 (dd, J=15.6, 7.2 Hz, 1H), 2.60 (dd, J=15.7, 7.6 Hz, 1H), 2.48-2.36 (m, 1H), 2.18-1.70 (m, 9H), 1.52-1.26 (m, 3H).

### Example 446

### Example 446A methyl (1r,4r)-4-(3-chloroanilino)-2′-{3-[(2-methylthieno[3,2-b]pyridin-7-yl)oxy]propyl}-2′,3′-dihydrospiro[cyclohexane-1,1′-indene]-4-carboxylate

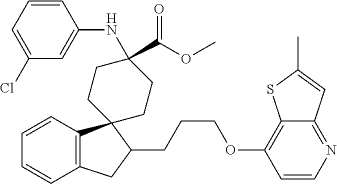

Using General procedure 31a and Preparation 9b (90 mg, 0.21 mmol, 1 eq) as the appropriate alcohol and 7-chloro-2-methyl-thieno[3,2-b]pyridine (46 mg, 0.25 mmol, 1.2 eq) as the appropriate aryl chloride, racemate Example 446A was obtained as an off-white foam (65 mg, 0.11 mmol, 54%). LRMS calculated for C₃₃H₃₅N₂O₃SCl: 574; found: 575 (M+H). ¹H NMR (400 MHz, DMSO-d₆) δ ppm: 8.42 (d, J=5.5 Hz, 1H), 7.41-7.35 (m, 1H), 7.24-7.12 (m, 4H), 7.05 (t, J=8.1 Hz, 1H), 6.93 (d, J=5.5 Hz, 1H), 6.59 (t, J=2.1 Hz, 1H), 6.57 (ddd, J=7.9, 2.0, 0.8 Hz, 1H), 6.45 (ddd, J=8.3, 2.3, 0.9 Hz, 1H), 6.31 (s, 1H), 4.36-4.23 (m, 2H), 3.66 (s, 3H), 3.05 (dd, J=15.7, 7.2 Hz, 1H), 2.59 (dd, J=15.7, 8.1 Hz, 1H), 2.56 (d, J=1.2 Hz, 3H), 2.48-2.38 (m, 1H), 2.21-1.66 (m, 9H), 1.50-1.22 (m, 3H).

### Example 446 (1r,4r)-4-(3-chloroanilino)-2′-{3-[(2-methylthieno[3,2-b]pyridin-7-yl)oxy]propyl}-2′,3′-dihydrospiro[cyclohexane-1,1′-indene]-4-carboxylic acid

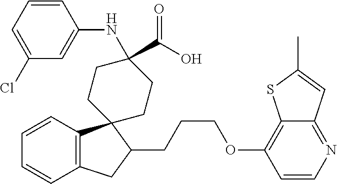

Using General procedure 33b and Example 446A (65 mg, 0.11 mmol, 1 eq) as the appropriate ester, racemate Example 446 was obtained as a white powder (15.6 mg, 0.03 mmol, 25%). LRMS calculated for C₃₂H₃₃N₂O₃SCl: 560; found: 561 (M+H). ¹H NMR (400 MHz, DMSO-d₆) δ 8.42 (d, J=5.4 Hz, 1H), 7.41-7.34 (m, 1H), 7.25-7.12 (m, 4H), 7.03 (t, J=8.1 Hz, 1H), 6.92 (d, J=5.5 Hz, 1H), 6.62 (t, J=2.1 Hz, 1H), 6.57-6.51 (m, 2H), 6.21 (br s, 1H), 4.34-4.24 (m, 2H), 3.04 (dd, J=15.7, 7.1 Hz, 1H), 2.59 (dd, J=15.7, 7.8 Hz, 1H), 2.56 (d, J=1.2 Hz, 3H), 2.47-2.37 (m, 1H), 2.20-1.67 (m, 9H), 1.52-1.38 (m, 2H), 1.38-1.25 (m, 1H).

### Example 447

### Example 447A methyl (1r,4r)-4-(3-chloroanilino)-2′-{3-[(furo[3,2-b]pyridin-7-yl)oxy]propyl}-2′,3′-dihydrospiro[cyclohexane-1,1′-indene]-4-carboxylate

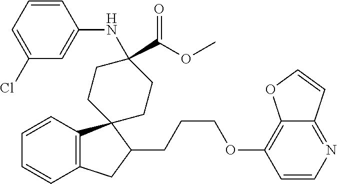

Using General procedure 31a and Preparation 9b (90 mg, 0.21 mmol, 1 eq) as the appropriate alcohol and 7-chlorofuro[3,2-b]pyridine, (39 mg, 0.25 mmol, 1.2 eq) as the appropriate aryl chloride, racemate Example 447A was obtained as an off-white foam (80 mg, 0.15 mmol, 72%). LRMS calculated for C₃₂H₃₃N₂O₄Cl: 544; found: 545 (M+H). ¹H NMR (400 MHz, DMSO-d₆) δ ppm: 8.34 (d, J=5.5 Hz, 1H), 8.17 (d, J=2.2 Hz, 1H), 7.43-7.36 (m, 1H), 7.25-7.12 (m, 3H), 7.08-6.99 (m, 3H), 6.60-6.54 (m, 2H), 6.48-6.43 (m, 1H), 6.31 (s, 1H), 4.37-4.27 (m, 2H), 3.66 (s, 3H), 3.04 (dd, J=15.7, 7.1 Hz, 1H), 2.60 (dd, J=15.7, 7.7 Hz, 1H), 2.49-2.38 (m, 1H), 2.19-1.65 (m, 9H), 1.51-1.26 (m, 3H).

### Example 447 (1r,4r)-4-(3-chloroanilino)-2′-{3-[(furo[3,2-b]pyridin-7-yl)oxy]propyl}-2′,3′-dihydrospiro[cyclohexane-1,1′-indene]-4-carboxylic acid

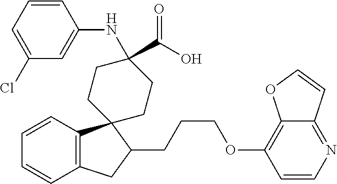

Using General procedure 33b and Example 447A (80 mg, 0.15 mmol, 1 eq) as the appropriate ester, racemate Example 447 was obtained as a white powder (23.6 mg, 0.04 mmol, 30%). LRMS calculated for C₃₁H₃₁N₂O₄Cl: 530; found: 531 (M+H). ¹H NMR (400 MHz, DMSO-d₆) δ ppm: 12.75 (br s, 1H), 8.34 (d, J=5.5 Hz, 1H), 8.16 (d, J=2.2 Hz, 1H), 7.42-7.34 (m, 1H), 7.25-7.12 (m, 3H), 7.08-6.99 (m, 3H), 6.61 (t, J=2.1 Hz, 1H), 6.57-6.51 (m, 2H), 6.21 (br s, 1H), 4.37-4.26 (m, 2H), 3.04 (dd, J=15.7, 7.2 Hz, 1H), 2.60 (dd, J=15.7, 7.7 Hz, 1H), 2.48-2.37 (m, 1H), 2.19-1.65 (m, 9H), 1.52-1.27 (m, 3H).

### Example 448 (1r,2′R,4R)-4-(3-chloroanilino)-2′-{3-[(3-methylpyridin-4-yl)oxy]propyl}-2′,3′-dihydrospiro[cyclohexane-1,1′-indene]-4-carboxylic acid

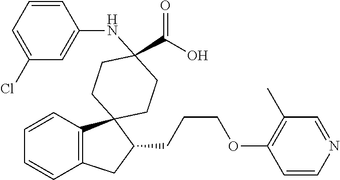 [SMILES: Cc1cnccc1OCCC[C@@H]1Cc2ccccc2[C@]12CC[C@@](Nc1cccc(Cl)c1)(C(=O)O)CC2]

To a solution of Preparation 9b1 (100 mg, 0.23 mmol, 1 eq) in THE (4 mL) was added NaH (23 mg, 0.58 mmol, 2.5 eq) and the reaction stirred for 5 min. 4-Chloro-3-methylpyridine (57 mg, 0.35 mmol, 1.5 eq) was added and the mixture was stirred at rt for 18 h. Additional NaH (23 mg, 0.58 mmol, 2.5 eq) was added and the mixture stirred at 70° C. for 18 h. DMF (4 mL) was introduced and additional 4-chloro-3-methylpyridine (57 mg, 0.35 mmol, 1.5 eq) and NaH (23 mg, 0.58 mmol, 2.5 eq) and heating continued at 70° C. for a further 48 h. The reaction was cooled to rt, partitioned between DCM and water, and the organic phase was dried (PTFE phase separator) and concentrated in vacuo. Purification by reverse phase automated flash chromatography (CombiFlash Rf, C18 4.3 g RediSep™ column) eluting with a gradient of 30-100% MeCN in water followed by automated flash chromatography (Combiflash Rf, 4 g, RediSep™ silica cartridge) eluting with a gradient of 0-10% MeOH in DCM and then preparative HPLC at pH 9 afforded Example 448 as a beige solid (6.38 mg, 0.01 mmol, 5%). LRMS calculated for C₃₀H₃₃N₂O₃Cl: 504; found: 505 (M+H). ¹H NMR (400 MHz, DMSO-d₆) δ ppm: 8.27 (d, J=5.6 Hz, 1H), 8.20 (s, 1H), 7.43-7.35 (m, 1H), 7.24-7.11 (m, 3H), 7.03 (t, J=8.1 Hz, 1H), 6.96 (d, J=5.6 Hz, 1H), 6.63 (t, J=2.1 Hz, 1H), 6.56-6.50 (m, 2H), 6.21 (bs, 1H), 4.16-4.04 (m, 2H), 3.02 (dd, J=15.7, 7.2 Hz, 1H), 2.57 (dd, J=15.7, 7.8 Hz, 1H), 2.47-2.36 (m, 1H), 2.17-1.66 (m, 12H), 1.51-1.24 (m, 3H).

### Example 449

### Example 449A methyl (1r,2′S,4S)-4-(3-chloroanilino)-2′-{3-[(3-methylpyridin-4-yl)oxy]propyl}-2′,3′-dihydrospiro[cyclohexane-1,1′-indene]-4-carboxylate

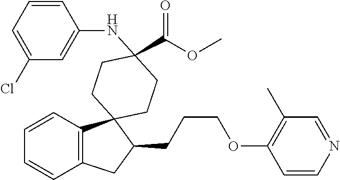 [SMILES: COC(=O)[C@]1(Nc2cccc(Cl)c2)CC[C@@]2(CC1)c1ccccc1C[C@@H]2CCCOc1ccncc1C]

Using General procedure 31a and Preparation 9b2 (100 mg, 0.23 mmol, 1 eq) as the appropriate alcohol and 4-bromo-3-methylpyridine×HCl (58 mg, 0.28 mmol, 1.2 eq) as the appropriate aryl halide, Example 449A was obtained as a beige solid (108 mg, 0.21 mmol, 89%). LRMS calculated for C₃₁H₃₅N₂O₃Cl: 518; found: 519 (M+H). ¹H NMR (400 MHz, DMSO-d₆) δ ppm: 8.27 (d, J=5.5 Hz, 1H), 8.20 (s, 1H), 7.43-7.36 (m, 1H), 7.24-7.12 (m, 3H), 7.06 (t, J=8.1 Hz, 1H), 6.96 (d, J=5.7 Hz, 1H), 6.62-6.54 (m, 2H), 6.47-6.41 (m, 1H), 6.33 (s, 1H), 4.17-4.03 (m, 2H), 3.66 (s, 3H), 3.03 (dd, J=15.6, 7.2 Hz, 1H), 2.58 (dd, J=15.7, 8.1 Hz, 1H), 2.49-2.39 (m, 1H), 2.17-1.66 (m, 12H), 1.51-1.41 (m, 1H), 1.41-1.25 (m, 2H).

### Example 449 (1r,2′S,4S)-4-(3-chloroanilino)-2′-{3-[(3-methylpyridin-4-yl)oxy]propyl}-2′,3′-dihydrospiro[cyclohexane-1,1′-indene]-4-carboxylic acid

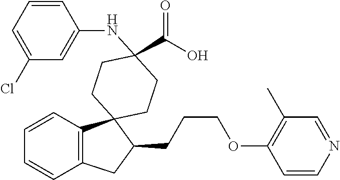 [SMILES: Cc1cnccc1OCCC[C@H]1Cc2ccccc2[C@]12CC[C@@](Nc1cccc(Cl)c1)(C(=O)O)CC2]

To a solution of Example 449A (108 mg, 0.21 mmol, 1 eq) in MeOH (5 mL) was added LiOH×H₂O (70 mg, 1.66 mmol, 8 eq) and water (0.5 mL) and the mixture was heated at 100° C. for 30 min under microwave irradiation. The reaction was concentrated, dissolved in a minimum amount of water and acidified to pH 3 with dilute aq. HCl solution. The aq. phase was extracted with DCM and the organic extract was dried (MgSO₄) and concentrated in vacuo to afford Example 449 as a solid (39.39 mg, 0.08 mmol, 37%). LRMS calculated for C₃₀H₃₃N₂O₃Cl: 504; found: 505 (M+H). ¹H NMR (400 MHz, DMSO-d₆) δ ppm: 12.71 (br s, 1H), 8.27 (d, J=5.6 Hz, 1H), 8.20 (s, 1H), 7.43-7.35 (m, 1H), 7.24-7.12 (m, 3H), 7.05 (t, J=8.1 Hz, 1H), 6.96 (d, J=5.6 Hz, 1H), 6.62 (t, J=2.2 Hz, 1H), 6.57-6.50 (m, 2H), 6.23 (br s, 1H), 4.16-4.04 (m, 2H), 3.03 (dd, J=15.7, 7.2 Hz, 1H), 2.58 (dd, J=15.7, 8.1 Hz, 1H), 2.47-2.37 (m, 1H), 2.16-1.96 (m, 6H), 1.96-1.83 (m, 3H), 1.83-1.67 (m, 3H), 1.51-1.42 (m, 1H), 1.42-1.23 (m, 2H).

### Example 450

### Example 450A methyl (1r,4r)-4-(3-chloroanilino)-2′-{3-[(pyrimidin-5-yl)oxy]propyl}-2′,3′-dihydrospiro[cyclohexane-1,1′-indene]-4-carboxylate

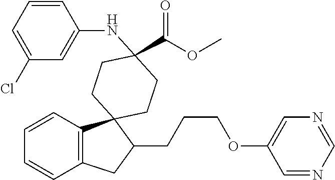

Using General procedure 30b and 5-hydroxypyrimidine (27 mg, 0.28 mmol, 1.5 eq) as the appropriate alcohol and Preparation 9b (80 mg, 0.19 mmol, 1 eq) as the appropriate indane, racemate Example 450A was obtained as an off-white glass (59 mg, 0.12 mmol, 62%). LRMS calculated for C₂₉H₃₂N₃O₃Cl: 505; found: 506 (M+H). ¹H NMR (400 MHz, DMSO-d₆) δ ppm: 8.80 (s, 1H), 8.56 (s, 2H), 7.42-7.35 (m, 1H), 7.24-7.12 (m, 3H), 7.06 (t, J=8.0 Hz, 1H), 6.61-6.54 (m, 2H), 6.45 (ddd, J=8.3, 2.2, 0.9 Hz, 1H), 6.33 (s, 1H), 4.19 (t, J=6.4 Hz, 2H), 3.66 (s, 3H), 3.02 (dd, J=15.6, 7.2 Hz, 1H), 2.58 (dd, J=15.7, 7.7 Hz, 1H), 2.48-2.39 (m, 1H), 2.18-1.84 (m, 6H), 1.81-1.62 (m, 3H), 1.51-1.42 (m, 1H), 1.42-1.22 (m, 2H).

### Example 450 (1r,4r)-4-(3-chloroanilino)-2′-{3-[(pyrimidin-5-yl)oxy]propyl}-2′,3′-dihydrospiro[cyclohexane-1,1′-indene]-4-carboxylic acid

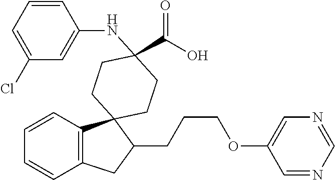

Using General procedure 33b and Example 450A (59 mg, 0.12 mmol, 1 eq) as the appropriate ester, racemate Example 450 was obtained as a white powder (33.7 mg, 0.07 mmol, 59%). LRMS calculated for C₂₈H₃₀N₃O₃Cl: 491; found: 492 (M+H). ¹H NMR (400 MHz, DMSO-d₆) δ ppm: 12.67 (s, 1H), 8.80 (s, 1H), 8.56 (s, 2H), 7.41-7.34 (m, 1H), 7.24-7.12 (m, 3H), 7.06 (t, J=8.1 Hz, 1H), 6.62 (t, J=2.1 Hz, 1H), 6.58-6.51 (m, 2H), 6.23 (br s, 1H), 4.19 (t, J=6.4 Hz, 2H), 3.02 (dd, J=15.6, 7.2 Hz, 1H), 2.58 (dd, J=15.7, 7.7 Hz, 1H), 2.47-2.37 (m, 1H), 2.17-1.83 (m, 6H), 1.82-1.62 (m, 3H), 1.52-1.24 (m, 3H).

### Example 451

### Example 451A methyl (1r,4r)-4-(3-chloroanilino)-2′-{3-[(pyrimidin-5-yl)oxy]propyl}-2′,3′-dihydrospiro[cyclohexane-1,1′-indene]-4-carboxylate

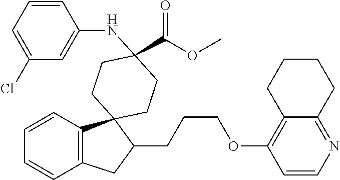

Using General procedure 31a and Preparation 9b (110 mg, 0.26 mmol, 1 eq) as the appropriate alcohol and 4-chloro-5,6,7,8-tetrahydroquinoline (52 mg, 0.31 mmol, 1.2 eq) as the appropriate aryl chloride, racemate Example 451A was obtained as a yellow glass (59 mg, 0.11 mmol, 41%). LRMS calculated for C₃₄H₃₉ClN₂O₃: 558; found: 559 (M+H). ¹H NMR (400 MHz, DMSO-d₆) δ ppm: 8.15 (d, J=5.6 Hz, 1H), 7.43-7.35 (m, 1H), 7.24-7.11 (m, 3H), 7.06 (t, J=8.0 Hz, 1H), 6.78 (d, J=5.7 Hz, 1H), 6.61-6.52 (m, 2H), 6.48-6.42 (m, 1H), 6.33 (s, 1H), 4.12-4.01 (m, 2H), 3.66 (s, 3H), 3.02 (dd, J=15.7, 7.2 Hz, 1H), 2.75-2.69 (m, 2H), 2.57 (dd, J=15.7, 8.0 Hz, 1H), 2.48-2.38 (m, 1H), 2.18-1.98 (m, 3H), 1.98-1.61 (m, 10H), 1.49-1.22 (m, 3H).

### Example 451 (1r,4r)-4-(3-chloroanilino)-2′-{3-[(5,6,7,8-tetrahydroquinolin-4-yl)oxy]propyl}-2′,3′-dihydrospiro[cyclohexane-1,1′-indene]-4-carboxylic acid

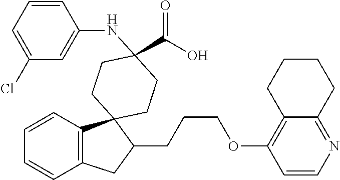

Using General procedure 33b and Example 451A (59 mg, 0.11 mmol, 1 eq) as the appropriate ester, racemate Example 451 was obtained as a white powder (19.4 mg, 0.04 mmol, 34%). LRMS calculated for C₃₃H₃₇ClN₂O₃: 544; found: 545 (M+H). ¹H NMR (400 MHz, DMSO-d₆) δ ppm: 8.15 (d, J=5.6 Hz, 1H), 7.42-7.34 (m, 1H), 7.25-7.11 (m, 3H), 7.04 (t, J=8.1 Hz, 1H), 6.78 (d, J=5.6 Hz, 1H), 6.62 (t, J=2.1 Hz, 1H), 6.58-6.51 (m, 2H), 6.23 (br s, 1H), 4.12-4.00 (m, 2H), 3.02 (dd, J=15.7, 7.2 Hz, 1H), 2.75-2.69 (m, 2H), 2.57 (dd, J=15.7, 7.8 Hz, 1H), 2.47-2.37 (m, 1H), 2.18-1.95 (m, 3H), 1.95-1.61 (m, 10H), 1.50-1.21 (m, 3H).

### Example 452

### Example 452A methyl (1r,4r)-4-(3-chloroanilino)-2′-{3-[(6,7-dihydro-5H-cyclopenta[b]pyridin-4-yl)oxy]propyl}-2′,3′-dihydrospiro[cyclohexane-1,1′-indene]-4-carboxylate

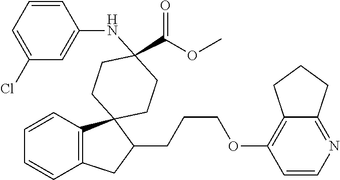

Using General procedure 31a and Preparation 9b (110 mg, 0.26 mmol, 1 eq) as the appropriate alcohol and 4-chloro-6,7-dihydro-5H-cyclopenta[b]pyridine (47 mg, 0.31 mmol, 1.2 eq) as the appropriate aryl chloride, racemate Example 452A was obtained as a yellow glass (75 mg, 0.14 mmol, 54%). LRMS calculated for C₃₃H₃₇ClN₂O₃: 544; found: 545 (M+H). ¹H NMR (400 MHz, DMSO-d₆) δ ppm: 8.16 (d, J=5.7 Hz, 1H), 7.43-7.35 (m, 1H), 7.24-7.12 (m, 3H), 7.06 (t, J=8.1 Hz, 1H), 6.78 (d, J=5.8 Hz, 1H), 6.62-6.55 (m, 2H), 6.47-6.42 (m, 1H), 6.33 (s, 1H), 4.17-4.04 (m, 2H), 3.66 (s, 3H), 3.02 (dd, J=15.7, 7.2 Hz, 1H), 2.84 (t, J=7.7 Hz, 2H), 2.75 (t, J=7.5 Hz, 2H), 2.57 (dd, J=15.7, 8.1 Hz, 1H), 2.49-2.38 (m, 1H), 2.18-2.07 (m, 1H), 2.07-1.65 (m, 10H), 1.50-1.21 (m, 3H).

### Example 452 (1r,4r)-4-(3-chloroanilino)-2′-{3-[(6,7-dihydro-5H-cyclopenta[b]pyridin-4-yl)oxy]propyl}-2′,3′-dihydrospiro[cyclohexane-1,1′-indene]-4-carboxylic acid

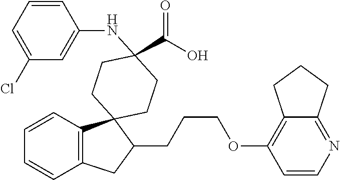

Using General procedure 33b and Example 452A (70 mg, 0.13 mmol, 1 eq) as the appropriate ester, racemate Example 452 was obtained as a white powder (20.2 mg, 0.04 mmol, 30%). LRMS calculated for C₃₂H₃₅ClN₂O₃: 530; found: 531 (M+H). ¹H NMR (400 MHz, DMSO-d₆) δ ppm: 12.72 (br s, 1H), 8.16 (d, J=5.7 Hz, 1H), 7.42-7.35 (m, 1H), 7.24-7.11 (m, 3H), 7.05 (t, J=8.1 Hz, 1H), 6.78 (d, J=5.8 Hz, 1H), 6.62 (t, J=2.1 Hz, 1H), 6.58-6.51 (m, 2H), 6.24 (br s, 1H), 4.16-4.04 (m, 2H), 3.02 (dd, J=15.7, 7.2 Hz, 1H), 2.84 (t, J=7.7 Hz, 2H), 2.75 (t, J=7.5 Hz, 2H), 2.57 (dd, J=15.6, 7.9 Hz, 1H), 2.47-2.37 (m, 1H), 2.17-1.65 (m, 11H), 1.50-1.21 (m, 3H).

### Example 453 (1r,4r)-4-(3-chloroanilino)-2′-{3-[(3-methyl-1H-indazol-4-yl)oxy]propyl}-2′,3′-dihydrospiro[cyclohexane-1,1′-indene]-4-carboxylic acid

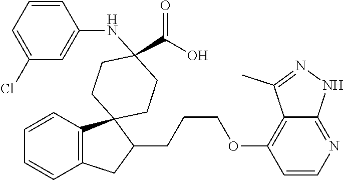

Using General procedure 30b and tert-butyl 4-hydroxy-3-methylindazole-1-carboxylate (87 mg, 0.35 mmol, 1.5 eq) as the appropriate alcohol and Preparation 9b (100 mg, 0.23 mmol, 1 eq) as the appropriate indane, an intermediate was obtained which was hydrolyzed according to General procedure 33b to yield racemate Example 453 as a white powder (40 mg, 0.07 mmol, 32%). LRMS calculated for C₃₂H₃₄ClN₃O₃: 543; found: 544 (M+H). ¹H NMR (400 MHz, DMSO-d₆) δ ppm: 12.51 (br s, 2H), 7.42-7.34 (m, 1H), 7.26-7.12 (m, 4H), 7.04 (t, J=8.0 Hz, 1H), 6.94 (d, J=8.2 Hz, 1H), 6.62 (t, J=2.1 Hz, 1H), 6.57-6.52 (m, 2H), 6.42 (d, J=7.6 Hz, 1H), 6.22 (br s, 1H), 4.10 (t, J=5.9 Hz, 2H), 3.06 (dd, J=15.6, 7.1 Hz, 1H), 2.61 (dd, J=15.7, 7.6 Hz, 1H), 2.49 (s, 3H), 2.48-2.39 (m, 1H), 2.19-1.72 (m, 9H), 1.52-1.32 (m, 3H).

### Example 454 and Example 455 Example 454A methyl (1r,4r)-4-(3-bromoanilino)-2′-(3-hydroxypropyl)spiro[cyclohexane-1,1′-indene]-4-carboxylate

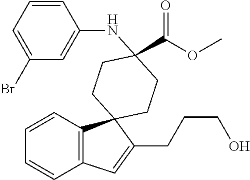 [SMILES: COC(=O)[C@]1(Nc2cccc(Br)c2)CC[C@]2(CC1)C(CCCO)=Cc1ccccc12]

Using General procedure 4 and (3-bromopropoxy)-tert-butyldimethylsilane (3.5 mL, 15.1 mmol, 1 eq) as the appropriate bromo compound, the zinc reagent was obtained as a 1 M solution. 3.32 mL (3.32 mmol, 2 eq) was coupled with Example 193B (894 mg, 1.66 mmol, 1 eq) as described in General procedure 27a, then it was treated as described in General procedure 29 to yield Example 454A as an orange solid (404 mg, 0.86 mmol, 52%). LRMS calculated for C₂₅H₂₈BrNO₃: 469; found: 470 (M+H). ¹H NMR (400 MHz, DMSO-d₆) δ ppm: 7.65 (d, J=7.6 Hz, 1H), 7.29 (dd, J=7.4, 1.3 Hz, 1H), 7.23 (td, J=7.4, 0.9 Hz, 1H), 7.11 (td, J=7.4, 1.4 Hz, 1H), 7.04 (t, J=8.1 Hz, 1H), 6.81 (t, J=2.1 Hz, 1H), 6.73 (ddd, J=7.8, 1.9, 0.8 Hz, 1H), 6.51 (ddd, J=8.2, 2.4, 0.8 Hz, 1H), 6.48-6.43 (m, 2H), 4.50 (t, J=5.1 Hz, 1H), 3.69 (s, 3H), 3.54-3.48 (m, 2H), 2.48-2.36 (m, 2H), 2.28-2.07 (m, 6H), 1.83-1.74 (m, 2H), 0.94-0.84 (m, 2H).

### Example 454B methyl (1r,4r)-4-(3-bromoanilino)-2′-{3-[(thieno[3,2-b]pyridin-7-yl)oxy]propyl}spiro[cyclohexane-1,1′-indene]-4-carboxylate

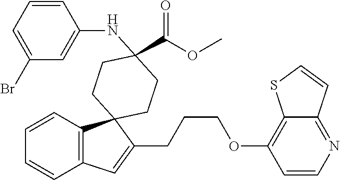 [SMILES: COC(=O)[C@]1(Nc2cccc(Br)c2)CC[C@]2(CC1)C(CCCOc1ccnc3ccsc13)=Cc1ccccc12]

To a solution of Example 454A (41.5 mg, 0.09 mmol, 1 eq) in THE (2 mL), cooled to 0° C., was added thieno[3,2-b]pyridine-7-ol (20 mg, 0.13 mmol, 1.5 eq) and PPh₃ (35 mg, 0.13 mmol, 1.5 eq) followed by DIAD (26 μL, 0.13 mmol, 1.5 eq) and the mixture was stirred at rt for 18 h.

The reaction was partitioned between DCM and water, and the organic phase was washed with brine, dried (MgSO₄) and concentrated in vacuo. Purification by automated flash column chromatography (CombiFlash Rf, 12 g RediSep™ silica cartridge) eluting with a gradient of 0-80% EtOAc in heptane afforded Example 454B as a white solid (38 mg, 0.06 mmol, 71%). LRMS calculated for C₃₂H₃₁BrN₂O₃S: 602; found: 603 (M+H). ¹H NMR (400 MHz, DMSO-d₆) δ ppm: 8.52 (d, J=5.4 Hz, 1H), 7.93 (d, J=5.4 Hz, 1H), 7.66 (d, J=7.5 Hz, 1H), 7.50 (d, J=5.4 Hz, 1H), 7.31 (d, J=7.4 Hz, 1H), 7.24 (t, J=7.4 Hz, 1H), 7.13 (t, J=7.5 Hz, 1H), 7.07-6.99 (m, 2H), 6.81 (t, J=2.1 Hz, 1H), 6.74 (dd, J=7.8, 1.8 Hz, 1H), 6.59 (s, 1H), 6.53-6.43 (m, 2H), 4.39 (t, J=6.2 Hz, 2H), 3.69 (s, 3H), 2.49-2.35 (m, 4H), 2.27-2.12 (m, 6H), 0.95-0.85 (m, 2H).

### Example 454 (1r,4r)-4-(3-bromoanilino)-2′-{3-[(thieno[3,2-b]pyridin-7-yl)oxy]propyl}spiro[cyclohexane-1,1′-indene]-4-carboxylic acid

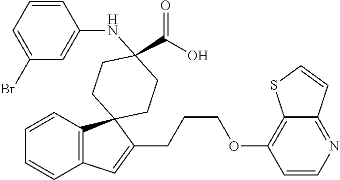 [SMILES: O=C(O)[C@]1(Nc2cccc(Br)c2)CC[C@]2(CC1)C(CCCOc1ccnc3ccsc13)=Cc1ccccc12]

and

### Example 455 (1r,4r)-4-anilino-2′-{3-[(thieno[3,2-b]pyridin-7-yl)oxy]propyl}spiro[cyclohexane-1,1′-indene]-4-carboxylic acid

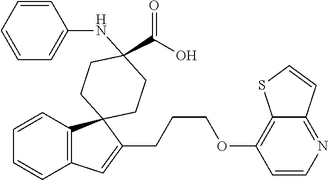 [SMILES: O=C(O)[C@]1(Nc2ccccc2)CC[C@]2(CC1)C(CCCOc1ccnc3ccsc13)=Cc1ccccc12]

Using General procedure 33b and Example 454B (38 mg, 0.06 mmol, 1 eq) as the appropriate ester, hydrolysis and partial debromination occurred. The products were separated via prep HPLC purification at pH 9. Example 454 was obtained as an off-white solid (17.2 mg, 0.03 mmol, 46%). LRMS calculated for C₃₁H₂₉BrN₂O₃S: 588; found: 589 (M+H). ¹H NMR (400 MHz, DMSO-d₆) δ ppm: 8.52 (d, J=5.4 Hz, 1H), 7.94 (d, J=5.4 Hz, 1H), 7.68 (d, J=7.6 Hz, 1H), 7.50 (d, J=5.4 Hz, 1H), 7.30 (dd, J=7.5, 1.2 Hz, 1H), 7.26-7.20 (m, 1H), 7.12 (td, J=7.5, 1.4 Hz, 1H), 7.04-6.98 (m, 2H), 6.81 (t, J=2.1 Hz, 1H), 6.70 (dd, J=7.8, 1.7 Hz, 1H), 6.62-6.56 (m, 2H), 6.31 (br s, 1H), 4.39 (t, J=6.3 Hz, 2H), 2.49-2.34 (m, 4H), 2.26-2.09 (m, 6H), 0.93-0.84 (m, 2H).

Example 455 was obtained as an off-white solid (5.15 mg, 0.01 mmol, 16%). LRMS calculated for C₃₁H₃₀N₂O₃S: 510; found: 511 (M+H). ¹H NMR (400 MHz, DMSO-d₆) δ ppm: 8.52 (d, J=5.4 Hz, 1H), 7.96 (d, J=5.4 Hz, 1H), 7.71 (d, J=7.5 Hz, 1H), 7.50 (d, J=5.4 Hz, 1H), 7.29 (dd, J=7.4, 1.2 Hz, 1H), 7.22 (t, J=7.3 Hz, 1H), 7.11 (td, J=7.5, 1.4 Hz, 1H), 7.07-7.00 (m, 3H), 6.66-6.60 (m, 2H), 6.58-6.49 (m, 2H), 4.39 (t, J=6.3 Hz, 2H), 2.49-2.34 (m, 4H), 2.25-2.12 (m, 6H), 0.90-0.81 (m, 2H).

### Example 456 (1r,4r)-4-(3-bromoanilino)-2′-{3-[(5-methylpyrimidin-4-yl)oxy]propyl}spiro[cyclohexane-1,1′-indene]-4-carboxylic acid

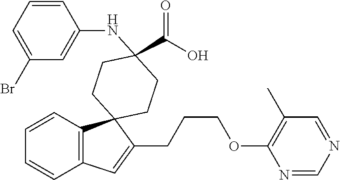 [SMILES: Cc1cncnc1OCCCC1=Cc2ccccc2[C@]12CC[C@@](Nc1cccc(Br)c1)(C(=O)O)CC2]

Using General procedure 31b and Example 454A (100 mg, 0.21 mmol, 1 eq) as the appropriate alcohol and 4-chloro-5-methylpyrimidine (41 mg, 0.32 mmol, 1.5 eq) as the appropriate aryl chloride, the desired intermediate was obtained. To a solution of the intermediate in MeOH (4 mL) was added a solution of LiOH×H₂O (36 mg, 0.85 mmol, 4 eq) in water (0.4 mL) and the mixture was heated at 110° C. for 90 min under microwave irradiation. The reaction was allowed to cool to rt, concentrated and the aq. phase acidified to pH 3 with 1 N aq. HCl solution. The mixture was extracted with DCM and the organic phase was washed with brine, dried (MgSO₄) and concentrated in vacuo. Purification by preparative HPLC at pH 4 afforded Example 456 as an off-white solid (28.4 mg, 0.05 mmol, 24%). LRMS calculated for C₂₉H₃₀BrN₃O₃: 547; found: 548 (M+H). ¹H NMR (400 MHz, DMSO-d₆) δ ppm: 12.81 (br s, 1H), 8.60 (s, 1H), 8.31 (s, 1H), 7.66 (d, J=7.6 Hz, 1H), 7.30 (dd, J=7.4, 1.3 Hz, 1H), 7.23 (td, J=7.4, 0.9 Hz, 1H), 7.12 (td, J=7.4, 1.4 Hz, 1H), 7.03 (t, J=8.0 Hz, 1H), 6.80 (t, J=2.1 Hz, 1H), 6.71 (dd, J=7.8, 1.7 Hz, 1H), 6.59-6.54 (m, 2H), 6.34 (br s, 1H), 4.46 (t, J=6.3 Hz, 2H), 2.45-2.32 (m, 4H), 2.21-2.06 (m, 6H), 2.04 (s, 3H), 0.92-0.82 (m, 2H).

### Example 457

### Example 457A methyl (1r,4r)-4-(3-chloroanilino)-2′-{3-[(2-methylpyridin-4-yl)oxy]propyl}-2′,3′-dihydrospiro[cyclohexane-1,1′-indene]-4-carboxylate

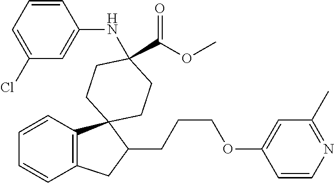

Using General procedure 31a and Preparation 9b (100 mg, 0.23 mmol, 1 eq) as the appropriate alcohol and 4-chloro-2-methylpyridine (31 μL, 0.28 mmol, 1.2 eq) as the appropriate aryl chloride, racemate Example 457A was obtained as a yellow glass (89 mg, 0.17 mmol, 74%). LRMS calculated for C₃₁H₃₅ClN₂O₃: 518; found: 519 (M+H). ¹H NMR (400 MHz, DMSO-d₆) δ ppm: 8.21 (d, J=5.8 Hz, 1H), 7.42-7.35 (m, 1H), 7.25-7.12 (m, 3H), 7.06 (t, J=8.0 Hz, 1H), 6.82 (d, J=2.4 Hz, 1H), 6.76 (dd, J=5.8, 2.5 Hz, 1H), 6.62-6.55 (m, 2H), 6.47-6.43 (m, 1H), 6.32 (s, 1H), 4.10-4.03 (m, 2H), 3.66 (s, 3H), 3.01 (dd, J=15.7, 7.2 Hz, 1H), 2.58 (dd, J=15.7, 8.0 Hz, 1H), 2.48-2.39 (m, 1H), 2.39 (s, 3H), 2.18-1.60 (m, 9H), 1.51-1.22 (m, 3H).

### Example 457 (1r,4r)-4-(3-chloroanilino)-2′-{3-[(2-methylpyridin-4-yl)oxy]propyl}-2′,3′-dihydrospiro[cyclohexane-1,1′-indene]-4-carboxylic acid

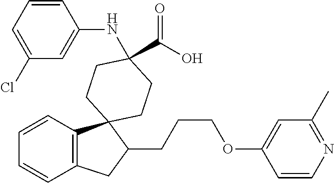

Using General procedure 33b and Example 457A (89 mg, 0.17 mmol, 1 eq) as the appropriate ester, racemate Example 457 was obtained as a white powder (6.7 mg, 0.01 mmol, 8%). LRMS calculated for C₃₀H₃₃ClN₂O₃: 504; found: 505 (M+H). ¹H NMR (400 MHz, DMSO-d₆) δ ppm: 12.67 (br s, 1H), 8.21 (d, J=5.7 Hz, 1H), 7.41-7.34 (m, 1H), 7.25-7.11 (m, 3H), 7.05 (t, J=8.1 Hz, 1H), 6.82 (d, J=2.5 Hz, 1H), 6.76 (dd, J=5.8, 2.4 Hz, 1H), 6.63 (t, J=2.1 Hz, 1H), 6.58-6.51 (m, 2H), 6.23 (br s, 1H), 4.06 (t, J=6.3 Hz, 2H), 3.01 (dd, J=15.7, 7.2 Hz, 1H), 2.57 (dd, J=15.8, 7.9 Hz, 1H), 2.47-2.38 (m, 1H), 2.39 (s, 3H), 2.18-1.60 (m, 9H), 1.51-1.21 (m, 3H).

### Example 458

### Example 458A methyl (1r,4r)-2′-(3-{[1-(benzenesulfonyl)-3-methyl-1H-pyrrolo[2,3-b]pyridin-4-yl]oxy}propyl)-4-(3-chloroanilino)-2′,3′-dihydrospiro[cyclohexane-1,1′-indene]-4-carboxylate

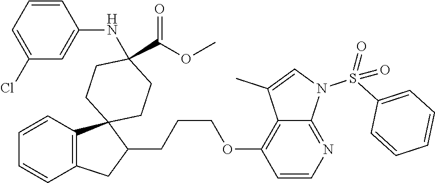

Using General procedure 31a and Preparation 9b (100 mg, 0.23 mmol, 1 eq) as the appropriate alcohol and 1-(benzenesulfonyl)-4-chloro-3-methylpyrrolo[2,3-b]pyridine (86 mg, 0.28 mmol, 1.2 eq) as the appropriate aryl chloride, racemate Example 458A was obtained as a cream foam (110 mg, 0.16 mmol, 67%). LRMS calculated for C₃₉H40ClN₃05S: 697; found: 698 (M+H). ¹H NMR (400 MHz, DMSO-d₆) δ ppm: 8.17 (d, J=5.6 Hz, 1H), 8.07-8.01 (m, 2H), 7.73-7.67 (m, 1H), 7.63-7.57 (m, 2H), 7.44-7.35 (m, 2H), 7.23-7.11 (m, 3H), 7.04 (t, J=8.3 Hz, 1H), 6.84 (d, J=5.7 Hz, 1H), 6.59-6.54 (m, 2H), 6.44-6.39 (m, 1H), 6.29 (s, 1H), 4.21-4.10 (m, 2H), 3.65 (s, 3H), 3.03 (dd, J=15.7, 7.2 Hz, 1H), 2.56 (dd, J=15.7, 8.0 Hz, 1H), 2.49-2.39 (m, 1H), 2.24 (d, J=1.3 Hz, 3H), 2.15-1.97 (m, 3H), 1.95-1.66 (m, 6H), 1.49-1.22 (m, 3H).

### Example 458 (1r,4r)-4-(3-chloroanilino)-2′-{3-[(3-methyl-1H-pyrrolo[2,3-b]pyridin-4-yl)oxy]propyl}-2′,3′-dihydrospiro[cyclohexane-1,1′-indene]-4-carboxylic acid

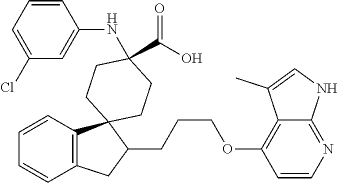

Using General procedure 33b and Example 458A (110 mg, 0.16 mmol, 1 eq) as the appropriate ester, racemate Example 458 was obtained as a white powder (35.5 mg, 0.07 mmol, 41%). LRMS calculated for C₃₂H₃₄ClN₃O₃: 543; found: 544 (M+H). ¹H NMR (400 MHz, DMSO-d₆) δ ppm: 12.69 (br s, 1H), 11.93 (s, 1H), 8.24 (d, J=6.4 Hz, 1H), 7.42-7.35 (m, 1H), 7.25-7.12 (m, 4H), 7.05 (t, J=8.1 Hz, 1H), 6.92 (d, J=6.4 Hz, 1H), 6.61 (t, J=2.1 Hz, 1H), 6.57-6.49 (m, 2H), 6.21 (br s, 1H), 4.39-4.28 (m, 2H), 3.06 (dd, J=15.7, 7.1 Hz, 1H), 2.60 (dd, J=15.7, 7.7 Hz, 1H), 2.49-2.38 (m, 1H), 2.29 (d, J=1.0 Hz, 3H), 2.18-1.72 (m, 9H), 1.51-1.30 (m, 3H).

### Example 459 (1r,4r)-4-(3-chloroanilino)-2′-{3-[(6,7-dihydro-5H-cyclopenta[d]pyrimidin-4-yl)oxy]propyl}-2′,3′-dihydrospiro[cyclohexane-1,1′-indene]-4-carboxylic acid

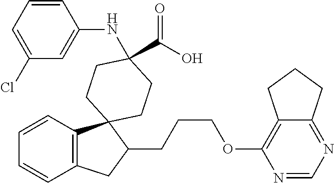

Using General procedure 31b and Preparation 9b (120 mg, 0.28 mmol, 1 eq) as the appropriate alcohol and 4-chloro-6,7-dihydro-5H-cyclopenta[d]pyrimidine (87 mg, 0.56 mmol, 2 eq) as the appropriate aryl chloride, racemate Example 459 was obtained as a beige powder (21.3 mg, 0.04 mmol, 14%). LRMS calculated for C₃₁H₃₄ClN₃O₃: 531; found: 532 (M+H). ¹H NMR (400 MHz, DMSO-d₆) δ ppm: 12.68 (br s, 1H), 8.54 (s, 1H), 7.41-7.33 (m, 1H), 7.24-7.11 (m, 3H), 7.06 (t, J=8.1 Hz, 1H), 6.62 (t, J=2.1 Hz, 1H), 6.58-6.50 (m, 2H), 6.23 (br s, 1H), 4.47-4.34 (m, 2H), 3.01 (dd, J=15.7, 7.2 Hz, 1H), 2.85 (t, J=7.8 Hz, 2H), 2.74 (t, J=7.6 Hz, 2H), 2.55 (dd, J=15.7, 7.8 Hz, 1H), 2.47-2.35 (m, 1H), 2.17-1.62 (m, 11H), 1.49-1.33 (m, 2H), 1.32-1.19 (m, 1H).

### Example 460 (1r,4r)-4-(3-chloroanilino)-2′-{3-[(5,6,7,8-tetrahydroquinazolin-4-yl)oxy]propyl}-2′,3′-dihydrospiro[cyclohexane-1,1′-indene]-4-carboxylic acid

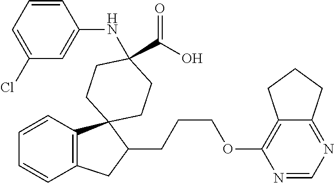

Using General procedure 31b and Preparation 9b (120 mg, 0.28 mmol, 1 eq) as the appropriate alcohol and 4-chloro-5,6,7,8-tetrahydroquinazoline (95 mg, 0.56 mmol, 2 eq) as the appropriate aryl chloride, racemate Example 460 was obtained as a cream powder (23.9 mg, 0.04 mmol, 16%). LRMS calculated for C₃₂H₃₆ClN₃O₃: 545; found: 546 (M+H). ¹H NMR (400 MHz, DMSO-d₆) δ ppm: 12.65 (br s, 1H), 8.46 (s, 1H), 7.41-7.32 (m, 1H), 7.24-7.10 (m, 3H), 7.05 (t, J=8.1 Hz, 1H), 6.61 (t, J=2.1 Hz, 1H), 6.57-6.50 (m, 2H), 6.23 (br s, 1H), 4.43-4.31 (m, 2H), 3.01 (dd, J=15.7, 7.2 Hz, 1H), 2.67 (t, J=6.2 Hz, 2H), 2.56 (dd, J=15.7, 8.1 Hz, 1H), 2.49-2.36 (m, 3H), 2.17-1.63 (m, 13H), 1.50-1.20 (m, 3H).

### Example 461 (1r,4r)-4-(3-chloroanilino)-2′-(3-{[5-(propan-2-yl)pyrimidin-4-yl]oxy}propyl)-2′,3′-dihydrospiro[cyclohexane-1,1′-indene]-4-carboxylic acid

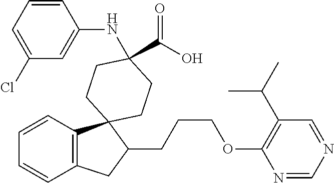

Using General procedure 31b and Preparation 9b (120 mg, 0.28 mmol, 1 eq) as the appropriate alcohol and 4-chloro-5-isopropylpyrimidine (88 mg, 0.56 mmol, 2 eq) as the appropriate aryl chloride, racemate Example 461 was obtained as a cream powder (10.9 mg, 0.02 mmol, 7%). LRMS calculated for C₃₁H₃₆ClN₃O₃: 533; found: 534 (M+H). ¹H NMR (400 MHz, DMSO-d₆) δ ppm: 12.66 (br s, 1H), 8.61 (s, 1H), 8.36 (s, 1H), 7.40-7.33 (m, 1H), 7.23-7.11 (m, 3H), 7.05 (t, J=8.1 Hz, 1H), 6.61 (t, J=2.1 Hz, 1H), 6.57-6.50 (m, 2H), 6.22 (br s, 1H), 4.45-4.35 (m, 2H), 3.07-2.93 (m, 2H), 2.57 (dd, J=15.7, 7.8 Hz, 1H), 2.47-2.36 (m, 1H), 2.18-1.62 (m, 9H), 1.50-1.36 (m, 2H), 1.34-1.22 (m, 1H), 1.16 (d, J=7.0 Hz, 3H), 1.15 (d, J=7.0 Hz, 3H).

### Example 463 (1r,4r)-4-(3-chloroanilino)-2′-(3-{[3-(trifluoromethyl)pyridin-4-yl]oxy}propyl)-2′,3′-dihydrospiro[cyclohexane-1,1′-indene]-4-carboxylic acid

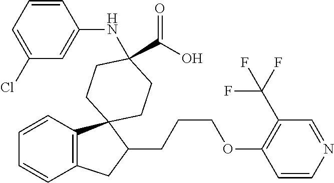

Using General procedure 31a and Preparation 9b (100 mg, 0.23 mmol, 1 eq) as the appropriate alcohol and 4-chloro-3-(trifluoromethyl)pyridine (51 mg, 0.28 mmol, 1.2 eq) as the appropriate aryl chloride, the desired intermediate was obtained. Using General procedure 33b, racemate Example 463 was obtained as a white powder (7.7 mg, 0.01 mmol, 6%). LRMS calculated for C₃₀H₃₀ClF₃N₂O₃: 558; found: 559 (M+H). ¹H NMR (400 MHz, DMSO-d₆) δ ppm: 8.70-8.65 (m, 2H), 7.40-7.35 (m, 1H), 7.33 (d, J=5.9 Hz, 1H), 7.23-7.11 (m, 3H), 7.04 (t, J=8.1 Hz, 1H), 6.62 (t, J=2.1 Hz, 1H), 6.56-6.51 (m, 2H), 6.16 (br s, 1H), 4.26 (t, J=6.3 Hz, 2H), 3.03 (dd, J=15.7, 7.2 Hz, 1H), 2.56 (dd, J=15.7, 7.8 Hz, 1H), 2.47-2.36 (m, 1H), 2.18-1.94 (m, 3H), 1.94-1.63 (m, 6H), 1.51-1.36 (m, 2H), 1.36-1.22 (m, 1H).

### Example 464

### Example 464A 4-chloro-3-cyclopropylpyridine

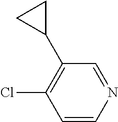

To a solution of 3-bromo-4-chloropyridine (350 mg, 1.82 mmol, 1 eq) in toluene (8 mL) and water (1 mL) was added potassium cyclopropyltrifluoroborate (296 mg, 2 mmol, 1.1 eq) and Cs₂CO₃ (1.48 g, 4.55 mmol, 2.5 eq). The mixture was sparged with N₂ (10 min) before the addition of cataCXium® A (32.6 mg, 0.09 mmol, 0.05 eq) and Pd(OAc)₂ (16 mg, 0.07 mmol, 0.04 eq) and the mixture was heated at 100° C. for 18 h. The mixture was partitioned between DCM and sat. aq. NaHCO₃ solution, and the organic phase was washed with brine, dried (PTFE phase separator) and concentrated in vacuo. Purification by automated flash chromatography (CombiFlash Rf, 12 g RediSep™ silica cartridge) eluting with a gradient of 0-10% MeOH in DCM afforded Example 464A as a colourless oil (98 mg, 0.64 mmol, 35%). LRMS calculated for C₈H₈ClN: 153; found: 154 (M+H). ¹H NMR (400 MHz, DMSO-d₆) δ ppm: 8.36 (d, J=5.3 Hz, 1H), 8.29 (s, 1H), 7.50 (dd, J=5.3, 0.5 Hz, 1H), 2.12-2.04 (m, 1H), 1.07-1.01 (m, 2H), 0.87-0.81 (m, 2H).

### Example 464 (1r,4r)-4-(3-chloroanilino)-2′-{3-[(3-cyclopropylpyridin-4-yl)oxy]propyl}-2′,3′-dihydrospiro[cyclohexane-1,1′-indene]-4-carboxylic acid

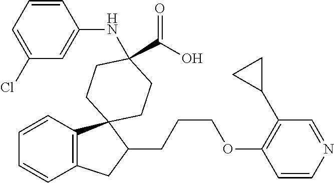

Using General procedure 31a and Preparation 9b (110 mg, 0.26 mmol, 1 eq) as the appropriate alcohol, and Example 464A (47 mg, 0.31 mmol, 1.2 eq) as the appropriate aryl chloride, an intermediate was obtained which was hydrolyzed according to General procedure 33b to obtain Example 464 as a white powder (8.9 mg, 0.017 mmol, 6%). LRMS calculated for C₃₂H₃₅ClN₂O₃: 530; found: 531 (M+H). ¹H NMR (400 MHz, DMSO-d₆) δ ppm: 8.23 (d, J=5.6 Hz, 1H), 7.98 (s, 1H), 7.41-7.34 (m, 1H), 7.25-7.11 (m, 3H), 7.05 (t, J=8.1 Hz, 1H), 6.94 (d, J=5.6 Hz, 1H), 6.62 (t, J=2.2 Hz, 1H), 6.58-6.50 (m, 2H), 6.22 (br s, 1H), 4.16-4.05 (m, 2H), 3.04 (dd, J=15.6, 7.2 Hz, 1H), 2.58 (dd, J=15.7, 7.6 Hz, 1H), 2.47-2.37 (m, 1H), 2.20-1.68 (m, 10H), 1.51-1.25 (m, 3H), 0.84-0.74 (m, 2H), 0.70-0.63 (m, 2H).

### Example 465

### Example 465A 4-chloro-5-cyclopropylpyrimidine

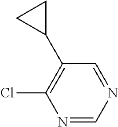

To a solution of 5-bromo-4-chloropyrimidine (350 mg, 1.81 mmol, 1 eq) in toluene (8 mL) and water (1 mL) was added potassium cyclopropyltrifluoroborate (296 mg, 2 mmol, 1.1 eq) and Cs₂CO₃ (1.48 g, 4.55 mmol, 2.5 eq). The mixture was sparged with N₂ (10 min) before the addition of cataCXium© A (33 mg, 0.09 mmol, 0.05 eq) and Pd(OAC)₂ (16 mg, 0.07 mmol, 0.04 eq) and the mixture was heated at 100° C. for 18 h. The mixture was partitioned between DCM and sat. aq. NaHCO₃ solution, and the organic phase was washed with brine, dried (PTFE phase separator) and concentrated in vacuo. Purification by automated flash chromatography (CombiFlash Rf, 12 g RediSep™ silica cartridge) eluting with a gradient of 0-10% MeOH in DCM afforded Example 465A as a yellow oil (78 mg, 0.5 mmol, 28%). LRMS calculated for C₇H7ClN₂: 154; found: 155 (M+H). ¹H NMR (400 MHz, DMSO-d₆) δ ppm: 8.86 (s, 1H), 8.52 (s, 1H), 2.10-2.01 (m, 1H), 1.11-1.04 (m, 2H), 0.94-0.88 (m, 2H).

### Example 465 (1r,4r)-4-(3-chloroanilino)-2′-{3-[(5-cyclopropylpyrimidin-4-yl)oxy]propyl}-2′,3′-dihydrospiro[cyclohexane-1,1′-indene]-4-carboxylic acid

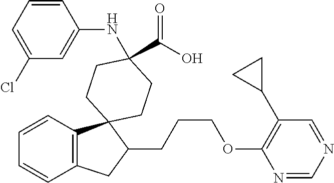

Using General procedure 31b and Preparation 9b (110 mg, 0.26 mmol, 1 eq) as the appropriate alcohol and Example 465A (80 mg, 0.51 mmol, 2 eq) as the appropriate aryl chloride, racemate Example 465 was obtained as a white powder (11.8 mg, 0.02 mmol, 9%). LRMS calculated for C₃₁H₃₄ClN₃O₃: 531; found: 532 (M+H). ¹H NMR (400 MHz, DMSO-d₆) δ ppm: 12.72 (br s, 1H), 8.56 (s, 1H), 8.15 (s, 1H), 7.41-7.33 (m, 1H), 7.23-7.11 (m, 3H), 7.05 (t, J=8.1 Hz, 1H), 6.61 (t, J=2.1 Hz, 1H), 6.57-6.50 (m, 2H), 6.21 (br s, 1H), 4.46-4.36 (m, 2H), 3.02 (dd, J=15.7, 7.1 Hz, 1H), 2.57 (dd, J=15.8, 7.8 Hz, 1H), 2.46-2.36 (m, 1H), 2.19-1.65 (m, 10H), 1.50-1.35 (m, 2H), 1.35-1.22 (m, 1H), 0.87-0.77 (m, 2H), 0.77-0.70 (m, 2H).

### Example 466

### Example 466A ethyl 2-diazo-6-methyl-3-oxoheptanoate

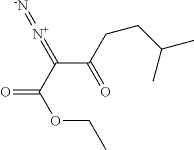

To a solution of ethyl 6-methyl-3-oxoheptanoate (2.5 g, 13.4 mmol, 1 eq) and 4-acetamidobenzenesulfonyl azide (3.29 g, 13.7 mmol, 1.02 eq) in MeCN (60 mL), cooled to 0° C., was added TEA (5.6 mL, 40.3 mmol, 3 eq). The mixture was allowed to warm to rt and stirred for 18 h. The mixture was concentrated in vacuo. The residue was triturated in Et₂O/heptane (1:1) and then filtered through celite. The filtrate was concentrated in vacuo. Purification by automated flash chromatography (CombiFlash Rf, 40 g RediSep™ silica cartridge) eluting with a gradient of 10-20% Et₂O in heptane afforded Example 466A as a yellow oil (2.31 g, 10.9 mmol, 81%). ¹H NMR (400 MHz, CDCl₃) δ ppm: 4.30 (q, J=7.1 Hz, 2H), 2.88-2.82 (m, 2H), 1.66-1.48 (m, 3H), 1.33 (t, J=7.1 Hz, 3H), 0.91 (d, J=6.5 Hz, 6H).

### Example 466B ethyl 2,2-dimethyl-5-oxocyclopentane-1-carboxylate

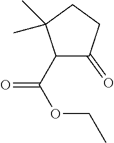

To a slurry of Rh₂(OAc)₄ (48 mg, 0.11 mmol, 0.01 eq) in DCM (40 mL), cooled to 0° C. was added a solution of Example 466A (2.31 g, 10.9 mmol, 1 eq) in DCM (10 mL). The mixture was warmed to rt and stirred for 5 h. The mixture was concentrated in vacuo and purification by automated flash chromatography (CombiFlash Rf, 24 g RediSep™ silica cartridge) eluting with a gradient of 0-12% EtOAc in heptane afforded racemate Example 466B as a colourless oil (1.72 g, 9.31 mmol, 86%). ¹H NMR (400 MHz, CDCl₃) δ ppm: 4.18 (q, J=7.1 Hz, 2H), 2.88 (d, J=0.9 Hz, 1H), 2.53-2.32 (m, 2H), 2.01 (ddd, J=12.8, 9.2, 5.9 Hz, 1H), 1.81-1.72 (m, 1H), 1.27 (t, J=7.1 Hz, 3H), 1.21 (s, 3H), 1.10 (s, 3H).

### Example 466C ethyl 2-amino-5,5-dimethylcyclopent-1-ene-1-carboxylate

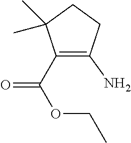

To a mixture of Example 466B (1.71 g, 9.28 mmol, 1 eq) in MeOH (20 mL) was added HCOONH₄ (2.93 g, 46.41 mmol, 5 eq) and the mixture was heated at 70° C. for 18 h. The mixture was allowed to cool to rt, concentrated and purified by automated flash chromatography (CombiFlash Rf, 24 g RediSep™ silica cartridge) eluting with a gradient of 0-15% EtOAc in heptane to afford Example 466C as a white solid (1.41 g, 7.69 mmol, 83%). ¹H NMR (400 MHz, DMSO-d₆) δ ppm: 6.71 (s, 2H), 4.03 (q, J=7.1 Hz, 2H), 2.37 (t, J=7.6 Hz, 2H), 1.55 (t, J=7.6 Hz, 2H), 1.19 (t, J=7.1 Hz, 3H), 1.13 (s, 6H).

### Example 466D 5,5-dimethyl-3,5,6,7-tetrahydro-4H-cyclopenta[d]pyrimidin-4-one

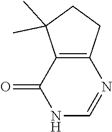

A solution of Example 466C (1.41 g, 7.69 mmol, 1 eq), HCOONH₄ (2.43 g, 38.5 mmol, 5 eq) and formamide (4.61 mL, 115 mmol, 15 eq) were heated at 150° C. for 8 h. The mixture was allowed to cool to rt and partitioned between water and DCM/iPrOH (5:1), and the organic phase was washed with brine, dried (MgSO₄) and concentrated in vacuo. Purification by automated flash chromatography (CombiFlash Rf, 24 g RediSep™ silica cartridge) eluting with a gradient of 20-100% EtOAc in heptane afforded Example 466D as a white solid (714 mg, 4.35 mmol, 57%). LRMS calculated for C₉H₁₂N₂O: 164; found: 165 (M+H). ¹H NMR (400 MHz, DMSO-d₆) δ ppm: 12.23 (s, 1H), 8.01 (s, 1H), 2.70 (dd, J=8.0, 7.2 Hz, 2H), 1.78 (dd, J=8.0, 7.2 Hz, 2H), 1.25 (s, 6H).

### Example 466E 4-chloro-5,5-dimethyl-6,7-dihydro-5H-cyclopenta[d]pyrimidine

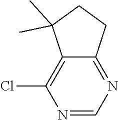

To a solution of Example 466D (714 mg, 4.35 mmol, 1 eq) in MeCN (10 mL) was added POCl₃ (3.24 mL, 34.8 mmol, 8 eq) and the mixture was heated at 80° C. for 48 h. The mixture was cooled to rt and added dropwise to a rapidly stirred solution of ice/water and stirred for 30 min. The mixture was basified with aq. NH₃ solution and extracted with DCM. The organic phase was washed with brine, dried (MgSO₄) and concentrated in vacuo. Purification by automated flash chromatography (CombiFlash Rf, 12 g RediSep™ silica cartridge) eluting with a gradient of 0-75% EtOAc in heptane afforded Example 466E as a yellow oil (289 mg, 1.58 mmol, 36%). LRMS calculated for C₉H₁₁ClN₂: 182; found: 183 (M+H). ¹H NMR (400 MHz, DMSO-d₆) δ ppm: 8.80 (s, 1H), 3.01 (t, J=7.7 Hz, 2H), 1.96 (t, J=7.7 Hz, 2H), 1.38 (s, 6H).

### Example 466 (1r,4r)-4-(3-chloroanilino)-2′-{3-[(5,5-dimethyl-6,7-dihydro-5H-cyclopenta[d]pyrimidin-4-yl)oxy]propyl}-2′,3′-dihydrospiro[cyclohexane-1,1′-indene]-4-carboxylic acid

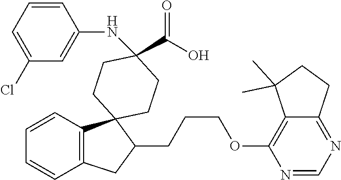

Using General procedure 31b and Preparation 9b (100 mg, 0.23 mmol, 1 eq) as the appropriate alcohol and Example 466E (77 mg, 0.5 mmol, 2 eq) as the appropriate aryl chloride, racemate Example 466 was obtained as a cream powder (23.4 mg, 0.04 mmol, 18%). LRMS calculated for C₃₃H₃ClN₃O₃: 559; found: 560 (M+H). ¹H NMR (400 MHz, DMSO-d₆) δ ppm: 12.7 (br s, 1H), 8.53 (s, 1H), 7.39-7.34 (m, 1H), 7.24-7.11 (m, 3H), 7.05 (t, J=8.1 Hz, 1H), 6.62 (t, J=2.1 Hz, 1H), 6.57-6.54 (m, 1H), 6.53-6.49 (m, 1H), 6.15 (br s, 1H), 4.47-4.34 (m, 2H), 3.03 (dd, J=15.6, 7.2 Hz, 1H), 2.85 (dd, J=8.2, 7.0 Hz, 2H), 2.56 (dd, J=15.6, 8.1 Hz, 1H), 2.45-2.36 (m, 1H), 2.19-1.64 (m, 11H), 1.49-1.34 (m, 2H), 1.34-1.22 (m, 1H), 1.22 (d, J=8.9 Hz, 6H).

### Example 467 (1r,4r)-4-(3-chloroanilino)-2′-{3-[(oxan-4-yl)oxy]propyl}-2′,3′-dihydrospiro[cyclohexane-1,1′-indene]-4-carboxylic acid

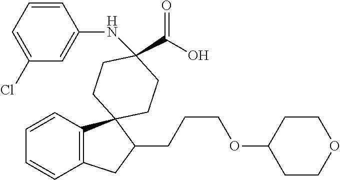

To a solution of Preparation 9c (47.8 mg, 0.09 mmol, 1 eq) in MeCN (2 mL) was added tetrahydro-2H-pyran-4-ol (90 μL, 0.94 mmol, 10 eq), followed by KOtBu (27 mg, 0.24 mmol, 2.5 eq) and the mixture was heated at 70° C. for 18 h. The mixture was concentrated in vacuo and suspended in a minimum amount of water, acidified with 2 M aq. HCl solution and the organics were extracted with DCM. The organic phase was washed with brine, dried (PTFE phase separator) and concentrated in vacuo. Purification by preparative HPLC afforded racemate Example 467 as a white solid (20 mg, 0.04 mmol, 43%). LRMS calculated for C₂₉H₃₆ClNO₄: 497; found: 498 (M+H). ¹H NMR (400 MHz, DMSO-d₆) δ ppm: 7.40-7.34 (m, 1H), 7.24-7.11 (m, 3H), 7.07 (t, J=8.1 Hz, 1H), 6.62 (t, J=2.1 Hz, 1H), 6.58-6.51 (m, 2H), 3.82-3.74 (m, 2H), 3.47-3.39 (m, 2H), 3.37-3.27 (m, 2H), 2.98 (dd, J=15.8, 7.2 Hz, 1H), 2.58-2.50 (m, 1H), 2.46-2.36 (m, 1H), 2.16-2.06 (m, 1H), 2.03-1.31 (m, 13H), 1.21-1.09 (m, 1H).

### Example 468

### Example 468A methyl 2-diazo-3-oxoheptanoate

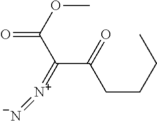

To a solution of methyl-3-oxoheptanoate (5.05 mL, 31.6 mmol, 1 eq) and 4-acetamidobenzenesulfonyl azide (7.75 g, 32.2 mmol, 1.02 eq) in MeCN (100 mL), cooled to 0° C., was added TEA (13.2 mL, 94.8 mmol, 3 eq). The mixture was allowed to warm to rt and stirred for 18 h. The mixture was concentrated in vacuo. The residue was triturated in Et₂O/heptane (1:1), filtered through celite and the filtrate was concentrated in vacuo. Purification by automated flash chromatography (CombiFlash Rf, 80 g RediSep™ silica cartridge) eluting with a gradient of 10-15% Et₂O in heptane afforded Example 468A as a yellow oil (5.22 g, 28.4 mmol, 90%). ¹H NMR (400 MHz, CDCl₃) δ ppm: 3.83 (s, 3H), 2.84 (t, J=7.5 Hz, 2H), 1.66-1.57 (m, 2H), 1.43-1.31 (m, 2H), 0.92 (t, J=7.4 Hz, 3H).

### Example 468B methyl 2-methyl-5-oxocyclopentane-1-carboxylate

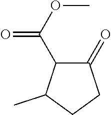

To a slurry of Rh₂(OAc)₄ (125 mg, 0.28 mmol, 0.01 eq) in DCM (80 mL), cooled to 0° C., was added a solution of Example 468A (5.2 g, 28.2 mmol, 1 eq) in DCM (20 mL). The mixture was warmed to rt and stirred for 3 h. The mixture was concentrated in vacuo and purification by automated flash chromatography (CombiFlash Rf, 80 g RediSep™ silica cartridge) eluting with a gradient of 0-16% EtOAc in heptane afforded a mixture of racemic diastereoisomers Example 468B as a colourless oil (2.83 g, 18.1 mmol, 64%). ¹H NMR (400 MHz, CDCl₃) δ ppm: 3.76 (s, 3H), 2.78 (dd, J=11.4, 1.0 Hz, 1H), 2.67-2.54 (m, 1H), 2.48-2.26 (m, 2H), 2.24-2.16 (m, 1H), 1.54-1.42 (m, 1H), 1.18 (d, J=6.6 Hz, 3H).

### Example 468C 2,5-dimethyl-3,5,6,7-tetrahydro-4H-cyclopenta[d]pyrimidin-4-one

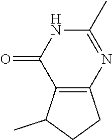

To a mixture of Example 468B (1.1 g, 7.04 mmol, 1 eq) in tBuOH (25 mL) was added acetamidine×HCl (1.00 g, 10.6 mmol, 1.5 eq) and KOtBu (2.37 g, 21.1 mmol, 3 eq) and the mixture was heated at reflux for 6 h. The mixture was allowed to cool to rt, filtered and washed with MeOH. The filtrate was concentrated in vacuo and purification by automated flash chromatography (CombiFlash Rf, 24 g RediSep™ silica cartridge) eluting with a gradient of 0-10% MeOH in DCM afforded racemate Example 468C as a white solid (586 mg, 3.57 mmol, 51%). LRMS calculated for C₉H₁₂N₂O: 164; found: 165 (M+H). ¹H NMR (400 MHz, DMSO-d₆) δ ppm: 12.16 (br s, 1H), 3.13-3.02 (m, 1H), 2.78-2.68 (m, 1H), 2.65-2.55 (m, 1H), 2.25 (s, 3H), 2.22-2.11 (m, 1H), 1.54-1.44 (m, 1H), 1.17 (d, J=6.8 Hz, 3H).

### Example 468D 4-chloro-2,5-dimethyl-6,7-dihydro-5H-cyclopenta[d]pyrimidine

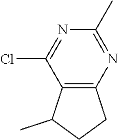

A solution of Example 468C (580 mg, 3.53 mmol, 1 eq) in POCl₃ (4.28 mL, 45.9 mmol, 13 eq) was heated at 90° C. for 3 h. The mixture was cooled to rt and added dropwise to a rapidly stirred solution of ice/water and stirred for 30 min. The mixture was basified with aq. NH₃ solution and extracted with DCM. The organic phase was washed with brine, dried (MgSO₄) and concentrated in vacuo. Purification by automated flash chromatography (CombiFlash Rf, 12 g RediSep™ silica cartridge) eluting with a gradient of 0-75% EtOAc in heptane afforded racemate Example 468D as a yellow oil (382 mg, 2.09 mmol, 59%). LRMS calculated for C₉H₁₁ClN₂: 182; found: 183 (M+H). ¹H NMR (400 MHz, DMSO-d₆) δ ppm: 3.38-3.28 (m, 1H), 3.08 (dt, J=17.9, 8.6 Hz, 1H), 2.87 (ddd, J=17.9, 9.4, 4.4 Hz, 1H), 2.56 (s, 3H), 2.35-2.24 (m, 1H), 1.76-1.68 (m, 1H), 1.25 (d, J=7.0 Hz, 3H).

### Example 468 (1r,4r)-4-(3-chloroanilino)-2′-{3-[(2,5-dimethyl-6,7-dihydro-5H-cyclopenta[d]pyrimidin-4-yl)oxy]propyl}-2′,3′-dihydrospiro[cyclohexane-1,1′-indene]-4-carboxylic acid

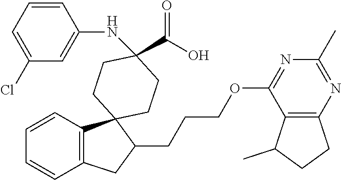

Using General procedure 31b and Preparation 9b (120 mg, 0.28 mmol, 1 eq) as the appropriate alcohol and Example 468D (81.9 mg, 0.45 mmol, 1.6 eq) as the appropriate aryl chloride, a racemic mixture of diastereoisomers, Example 468 was obtained as a yellow powder (70.9 mg, 0.13 mmol, 45%). LRMS calculated for C₃₃H₃₈ClN₃O₃: 559; found: 560 (M+H). ¹H NMR (400 MHz, DMSO-d₆) δ ppm: 12.66 (br s, 1H), 7.39-7.33 (m, 1H), 7.23-7.11 (m, 3H), 7.09-7.02 (m, 1H), 6.64-6.60 (m, 1H), 6.58-6.49 (m, 2H), 6.19 (br s, 1H), 4.51-4.28 (m, 2H), 3.22-3.13 (m, 1H), 3.08-2.97 (m, 1H), 2.93-2.81 (m, 1H), 2.80-2.68 (m, 1H), 2.61-2.50 (m, 1H), 2.47 (s, 3H), 2.47-2.36 (m, 1H), 2.28-1.93 (m, 4H), 1.93-1.61 (m, 6H), 1.60-1.20 (m, 4H), 1.15/1.13 (d, J=7.2 Hz, 3H).

### Example 469 (1r,4r)-2′-{3-[(1H-benzimidazol-4-yl)oxy]propyl}-4-(3-chloroanilino)-2′,3′-dihydrospiro[cyclohexane-1,1′-indene]-4-carboxylic acid

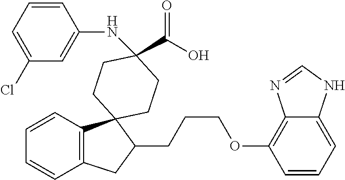

Using General procedure 30b and 1H-benzo[d]imidazol-4-ol (42 mg, 0.32 mmol, 1.5 eq) as the appropriate alcohol and Preparation 9b (90 mg, 0.21 mmol, 1 eq) as the appropriate indane afforded an intermediate which was hydrolyzed according to General procedure 33b to yield racemate Example 469 as a white powder (34.4 mg, 0.06 mmol, 40%). LRMS calculated for C₃₁H₃₂ClN₃O₃: 529; found: 530 (M+H). ¹H NMR (400 MHz, DMSO-d₆) δ ppm: 12.67 (br s, 1H), 12.58/12.40 (s, 1H), 8.10/8.06 (s, 1H), 7.43-7.34 (m, 1H), 7.27-7.00 (m, 6H), 6.81-6.65 (m, 1H), 6.63-6.51 (m, 3H), 6.21 (br s, 1H), 4.34-4.12 (m, 2H), 3.08-2.99 (m, 1H), 2.66-2.56 (m, 1H), 2.48-2.38 (m, 1H), 2.19-1.67 (m, 9H), 1.54-1.28 (m, 3H).

### Example 470 (1r,4r)-4-(3-chloroanilino)-2′-{3-[(2,3-dihydro-1H-indol-4-yl)oxy]propyl}-2′,3′-dihydrospiro[cyclohexane-1,1′-indene]-4-carboxylic acid

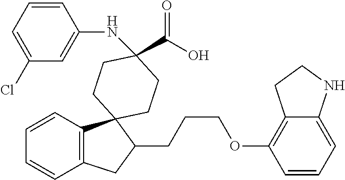

Using General procedure 30b and 4-hydroxyindoline (42 mg, 0.31 mmol, 1.5 eq) as the appropriate alcohol and Preparation 9b (90 mg, 0.21 mmol, 1 eq) as the appropriate indane afforded an intermediate which was hydrolyzed according to General procedure 33b to yield racemate Example 470 as a yellow powder (17.7 mg, 0.03 mmol, 20%). LRMS calculated for C₃₂H₃₅ClN₂O₃: 530; found: 531 (M+H). ¹H NMR (400 MHz, DMSO-d₆) δ ppm: 12.71 (br s, 1H), 7.42-7.35 (m, 1H), 7.24-7.12 (m, 3H), 7.10-7.03 (m, 2H), 6.62 (t, J=2.1 Hz, 1H), 6.61-6.50 (m, 4H), 6.25 (br s, 1H), 4.10-3.97 (m, 2H), 3.50 (t, J=8.3 Hz, 2H), 3.02 (dd, J=15.6, 7.2 Hz, 1H), 2.90 (t, J=8.2 Hz, 2H), 2.57 (dd, J=15.7, 7.9 Hz, 1H), 2.48-2.38 (m, 1H), 2.17-1.96 (m, 3H), 1.95-1.65 (m, 6H), 1.51-1.21 (m, 3H).

### Example 471 (1r,4r)-4-(3-chloroanilino)-2′-{3-[(3,4-dihydro-2H-1-benzopyran-4-yl)oxy]propyl}-2′,3′-dihydrospiro[cyclohexane-1,1′-indene]-4-carboxylic acid

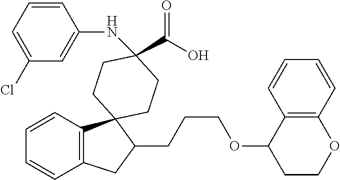

To a solution of Preparation 9c (45 mg, 0.09 mmol, 1 eq) in MeCN (2 mL) was added 4-chromanol (67 mg, 0.44 mmol, 5 eq), followed by KOtBu (25 mg, 0.22 mmol, 2.5 eq) and the mixture was heated at 70° C. for 18 h. The mixture was concentrated in vacuo and the solid was dissolved in minimal MeOH, then loaded onto a MeOH-wet PE-AX cartridge, washed successively with MeOH and DCM, eluted with 10% HCOOH in DCM, and concentrated in vacuo. Further purification by preparative HPLC afforded a racemic mixture of diastereoisomers Example 471 as a white solid (21.8 mg, 0.04 mmol, 45%). LRMS calculated for C₃₃H₃₆ClNO₄: 545; found: 546 (M+H). ¹H NMR (400 MHz, DMSO-d₆) δ ppm: 7.40-7.34 (m, 1H), 7.25-7.10 (m, 5H), 7.05 (t, J=8.1 Hz, 1H), 6.86-6.73 (m, 2H), 6.65-6.62 (m, 1H), 6.57-6.51 (m, 2H), 6.22 (br s, 1H), 4.40-4.35 (m, 1H), 4.22-4.06 (m, 2H), 3.67-3.47 (m, 2H), 2.97 (dd, J=15.7, 7.2 Hz, 1H), 2.58-2.51 (m, 1H), 2.45-2.36 (m, 1H), 2.17-1.88 (m, 7H), 1.78-1.32 (m, 6H), 1.25-1.11 (m, 1H).

### Example 472

### Example 472A methyl (5R)-2-amino-5-methylcyclopent-1-ene-1-carboxylate

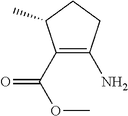

To a mixture of methyl (2R)-2-methyl-5-oxocyclopentane-1-carboxylate (1 g, 6.4 mmol, 1 eq) in MeOH (10 mL) was added NH₄HCO₂ (2.02 g, 32 mmol, 5 eq) and the mixture was heated at reflux for 6 h. The mixture was allowed to cool to rt, concentrated and purified by automated flash chromatography (CombiFlash Rf, 24 g RediSep™ silica cartridge) eluting with a gradient of 0-20% EtOAc in heptane to afford Example 472A as an off-white solid (894 mg, 5.76 mmol, 90%). LRMS calculated for C₈H₁₀N₂O: 155; found: 156 (M+H). ¹H NMR (400 MHz, DMSO-d₆) δ ppm: 6.74 (br s, 2H), 3.55 (s, 3H), 2.86-2.77 (m, 1H), 2.57-2.47 (m, 1H), 2.29 (ddd, J=16.8, 9.4, 4.1 Hz, 1H), 1.98-1.87 (m, 1H), 1.35-1.27 (m, 1H), 1.01 (d, J=6.6 Hz, 3H).

### Example 472B (5R)-5-methyl-3,5,6,7-tetrahydro-4H-cyclopenta[d]pyrimidin-4-one

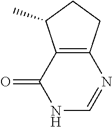

A solution of Example 472A (894 mg, 5.76 mmol, 1 eq), NH₄HCO₂ (1.82 g, 28.8 mmol, 5 eq) and formamide (1.15 mL, 28.8 mmol, 5 eq) was heated at 150° C. for 4 h. The mixture was allowed to cool to rt and partitioned between water and DCM/iPrOH (5:1). The organic phase was washed with brine, dried (MgSO₄) and concentrated in vacuo. Purification by automated flash chromatography (CombiFlash Rf, 12 g RediSep™ silica cartridge) eluting with a gradient of 0-10% MeOH in DCM afforded Example 472B as a yellow solid (135 mg, 0.9 mmol, 16%). LRMS calculated for C₉H₁₂N₂O: 150; found: 151 (M+H). ¹H NMR (400 MHz, DMSO-d₆) δ ppm: 12.26 (br s, 1H), 8.02 (s, 1H), 3.17-3.06 (m, 1H), 2.84-2.74 (m, 1H), 2.71-2.60 (m, 1H), 2.24-2.13 (m, 1H), 1.57-1.46 (m, 1H), 1.18 (d, J=6.9 Hz, 3H).

### Example 472C (5R)-4-chloro-5-methyl-6,7-dihydro-5H-cyclopenta[d]pyrimidine

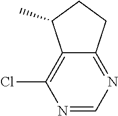

To Example 472B (130 mg, 0.87 mmol, 1 eq) was added POCl₃ (0.81 mL, 8.66 mmol, 10 eq) and the mixture was heated at 95° C. for 2 h. The mixture was cooled to rt and added dropwise to a rapidly stirred solution of ice/water and stirred for 30 min. The mixture was basified with aq. NH₃ solution and extracted with DCM. The organic phase was washed with brine, dried (PTFE phase separator) and concentrated in vacuo to afford Example 472C as a brown oil (138 mg, 0.82 mmol, 95%). LRMS calculated for C₈H₉ClN₂: 168; found: 169 (M+H). ¹H NMR (400 MHz, DMSO-d₆) δ ppm: 8.80 (s, 1H), 3.44-3.34 (m, 1H), 3.14 (dt, J=17.3, 8.5 Hz, 1H), 2.92 (ddd, J=18.0, 9.4, 4.4 Hz, 1H), 2.37-2.26 (m, 1H), 1.80-1.70 (m, 1H), 1.28 (d, J=7.1 Hz, 3H).

### Example 472 (1r,4R)-4-(3-chloroanilino)-2′-(3-{[(5R)-5-methyl-6,7-dihydro-5H-cyclopenta[d]pyrimidin-4-yl]oxy}propyl)-2′,3′-dihydrospiro[cyclohexane-1,1′-indene]-4-carboxylic acid

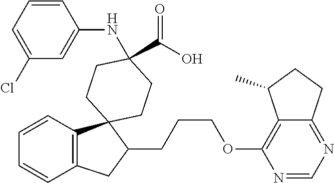

To an oven-dried flask was added Preparation 9b (84 mg, 0.19 mmol, 1 eq) and Example 472C (49.7 mg, 0.29 mmol, 1.5 eq) in DMF (2 mL) and the solution was cooled to 0° C. NaH (60% dispersion; 24 mg, 0.59 mmol, 3 eq) was added portionwise and the mixture was allowed to warm to rt and stirred for 72 h. The mixture was partitioned between DCM and water, and the organics were extracted with DCM. The combined organic extracts were washed with 1 M aq. HCl solution, brine, dried (PTFE phase separator) and concentrated in vacuo. Purification by reverse phase automated flash chromatography (CombiFlash Rf, C18 13 g RediSep column) eluting with a gradient of 30-100% MeCN in water afforded a diastereoisomer mixture Example 472 as a cream powder (22 mg, 0.04 mmol, 21%). LRMS calculated for C₃₂H₃₆ClN₃O₃: 545; found: 546 (M+H). ¹H NMR (400 MHz, DMSO-d₆) δ ppm: 12.70 (br s, 1H), 8.54 (s, 1H), 7.40-7.34 (m, 1H), 7.24-7.11 (m, 3H), 7.06/7.05 (t, J=8.1 Hz, 1H), 6.63-6.59 (m, 1H), 6.58-6.49 (m, 2H), 6.20 (br s, 1H), 4.52-4.30 (m, 2H), 3.28-3.15 (m, 1H), 3.06-2.97 (m, 1H), 2.96-2.85 (m, 1H), 2.83-2.71 (m, 1H), 2.60-2.50 (m, 1H), 2.47-2.36 (m, 1H), 2.28-1.94 (m, 4H), 1.94-1.61 (m, 6H), 1.61-1.50 (m, 1H), 1.49-1.20 (m, 3H), 1.17/1.15 (d, J=6.9 Hz, 3H).

### Example 473

### Example 473A 4-chloro-8-fluoro-5,6,7,8-tetrahydroquinoline

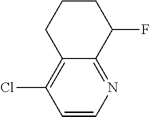 [SMILES: FC1CCCc2c(Cl)ccnc21]

To a solution of 4-chloro-5,6,7,8-tetrahydroquinolin-8-ol (500 mg, 2.72 mmol, 1 eq) in DCM (10 mL), cooled to −5° C., was added DAST (1.08 mL, 8.17 mmol, 3 eq). The mixture was slowly warmed to rt over 4 h. The mixture was cooled to 0° C., quenched by the dropwise addition of sat. aq. NH₄Cl solution and then basified to pH 11 with 2 M aq. NaOH solution. The organics were dried (PTFE phase separator) and concentrated in vacuo. Purification by reverse phase automated flash chromatography (CombiFlash Rf, C18 26 g RediSep column) eluting with a gradient of 10-60% MeCN in water afforded racemate Example 473A as a cream solid (78 mg, 0.42 mmol, 15%). LRMS calculated for C₉H₉ClFN: 185; found: 186 (M+H). ¹H NMR (400 MHz, DMSO-d₆) δ ppm: 8.47 (dd, 1H), 7.59 (dd, J=5.2, 2.0 Hz, 1H), 5.53 (dt, J=49.5, 3.5 Hz, 1H), 2.98-2.87 (m, 1H), 2.71-2.58 (m, 1H), 2.31-2.19 (m, 1H), 2.08-1.76 (m, 3H).

### Example 473B methyl (1r,4r)-4-(3-chloroanilino)-2′-{3-[(8-fluoro-5,6,7,8-tetrahydroquinolin-4-yl)oxy]propyl}-2′,3′-dihydrospiro[cyclohexane-1,1′-indene]-4-carboxylate

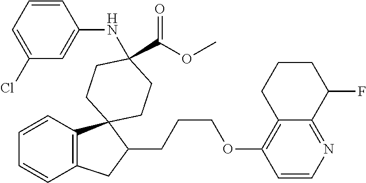

Using General procedure 31a and Preparation 9b (100 mg, 0.23 mmol, 1 eq) as the appropriate alcohol and Example 473A (52 mg, 0.28 mmol, 1.2 eq) as the appropriate aryl chloride, a racemic mixture of diastereoisomers Example 473B was obtained as a white glass (71 mg, 0.12 mmol, 53%). LRMS calculated for C₃₄H₃₈ClFN₂O₃: 576; found: 577 (M+H). ¹H NMR (400 MHz, DMSO-d₆) δ ppm: 8.34 (d, J=5.5 Hz, 1H), 7.43-7.35 (m, 1H), 7.25-7.12 (m, 3H), 7.08-6.97 (m, 2H), 6.61-6.51 (m, 2H), 6.48-6.42 (m, 1H), 6.33 (s, 1H), 5.52-5.34 (m, 1H), 4.18-4.05 (m, 2H), 3.66 (s, 3H), 3.03 (dd, J=15.7, 7.3 Hz, 1H), 2.81-2.69 (m, 1H), 2.58 (dd, J=15.6, 8.0 Hz, 1H), 2.49-2.31 (m, 2H), 2.25-1.65 (m, 13H), 1.51-1.23 (m, 3H).

### Example 473 (1r,4r)-4-(3-chloroanilino)-2′-{3-[(8-fluoro-5,6,7,8-tetrahydroquinolin-4-yl)oxy]propyl}-2′,3′-dihydrospiro[cyclohexane-1,1′-indene]-4-carboxylic acid

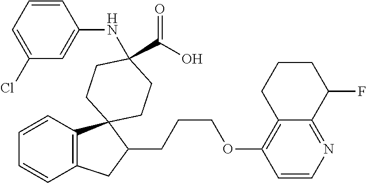

Using General procedure 33b and Example 473B (71 mg, 0.12 mmol, 1 eq) as the appropriate ester, a racemic mixture of diastereoisomers Example 473 was obtained as a white powder (34.4 mg, 0.06 mmol, 50%). LRMS calculated for C₃₃H₃₆ClFN₂O₃: 562; found: 563 (M+H). ¹H NMR (400 MHz, DMSO-d₆) δ ppm: 8.34 (d, J=5.5 Hz, 1H), 7.43-7.34 (m, 1H), 7.25-7.12 (m, 3H), 7.08-6.97 (m, 2H), 6.65-6.59 (m, 1H), 6.57-6.51 (m, 2H), 6.24 (br s, 1H), 5.52-5.34 (m, 1H), 4.19-4.05 (m, 2H), 3.03 (dd, J=15.6, 7.2 Hz, 1H), 2.81-2.70 (m, 1H), 2.58 (dd, J=15.7, 7.9 Hz, 1H), 2.48-2.31 (m, 2H), 2.25-1.63 (m, 13H), 1.52-1.22 (m, 3H).

### Example 474

### Example 474A methyl (1r,4r)-4-(3-chloroanilino)-2′-{3-[(8-hydroxy-5,6,7,8-tetrahydroquinolin-4-yl)oxy]propyl}-2′,3′-dihydrospiro[cyclohexane-1,1′-indene]-4-carboxylate

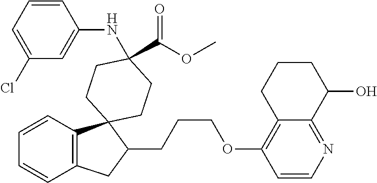

Using General procedure 31a and Preparation 9b (100 mg, 0.23 mmol, 1 eq) as the appropriate alcohol and 4-chloro-5,6,7,8-tetrahydro-quinolin-8-ol (52 mg, 0.28 mmol, 1.2 eq) as the appropriate aryl chloride and then further purification by SCX cartridge (2 g), washing successively with DCM and MeOH and eluting with 4:1 DCM/7M NH₃ in MeOH, a racemic mixture of diastereoisomers Example 474A was obtained as an off-white glass (90 mg, 0.16 mmol, 67%). LRMS calculated for C₃₄H₃₉ClN₂O₄: 574; found: 575 (M+H). ¹H NMR (400 MHz, DMSO-d₆) δ ppm: 8.27 (d, J=5.5 Hz, 1H), 7.43-7.36 (m, 1H), 7.25-7.12 (m, 3H), 7.06 (t, J=8.0 Hz, 1H), 6.87 (d, J=5.6 Hz, 1H), 6.61-6.51 (m, 2H), 6.48-6.43 (m, 1H), 6.33 (s, 1H), 5.00-4.96 (m, 1H), 4.52-4.46 (m, 1H), 4.16-4.02 (m, 2H), 3.66 (s, 3H), 3.03 (dd, J=15.7, 7.2 Hz, 1H), 2.65-2.51 (m, 2H), 2.49-2.37 (m, 2H), 2.20-1.55 (m, 13H), 1.51-1.22 (m, 3H).

### Example 474 (1r,4r)-4-(3-chloroanilino)-2′-{3-[(8-hydroxy-5,6,7,8-tetrahydroquinolin-4-yl)oxy]propyl}-2′,3′-dihydrospiro[cyclohexane-1,1′-indene]-4-carboxylic acid

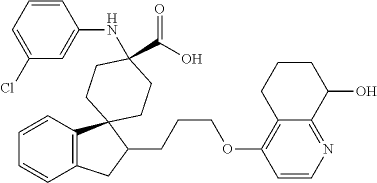

Using General procedure 33b and Example 474A (90 mg, 0.16 mmol, 1 eq) as the appropriate ester, a racemic mixture of diastereoisomers Example 474 was obtained as a white powder (39.2 mg, 0.07 mmol, 45%). LRMS calculated for C₃₃H₃₇ClN₂O₄: 560; found: 561 (M+H). ¹H NMR (400 MHz, DMSO-d₆) δ ppm: 8.27 (d, J=5.6 Hz, 1H), 7.42-7.35 (m, 1H), 7.24-7.12 (m, 3H), 7.05 (t, J=8.1 Hz, 1H), 6.87 (d, J=5.6 Hz, 1H), 6.63-6.59 (m, 1H), 6.58-6.51 (m, 2H), 6.23 (br s, 1H), 4.99 (br s, 1H), 4.52-4.46 (m, 1H), 4.16-4.01 (m, 2H), 3.03 (dd, J=15.7, 7.2 Hz, 1H), 2.66-2.51 (m, 2H), 2.49-2.36 (m, 2H), 2.18-1.55 (m, 13H), 1.51-1.23 (m, 3H).

### Example 475

### Example 475A methyl (1r,4r)-4-(3-chloroanilino)-2′-{3-[(7-hydroxy-6,7-dihydro-5H-cyclopenta[b]pyridin-4-yl)oxy]propyl}-2′,3′-dihydrospiro[cyclohexane-1,1′-indene]-4-carboxylate

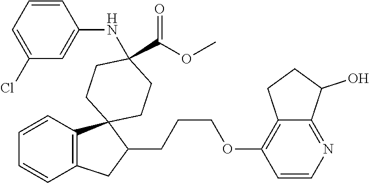

Using General procedure 31a and Preparation 9b (100 mg, 0.23 mmol, 1 eq) as the appropriate alcohol and 4-chloro-6,7-dihydro-5H-cyclopenta[b]pyridin-7-ol (48 mg, 0.28 mmol, 1.2 eq) as the appropriate aryl halide and then further purification by SCX cartridge (2 g), washing successively with DCM and MeOH and eluting with 4:1 DCM/7M NH₃ in MeOH, a racemic mixture of diastereoisomers Example 475A was obtained as a beige glass (58 mg, 0.1 mmol, 44%). LRMS calculated for C₃₃H₃₇ClN₂O₄: 560; found: 561 (M+H). ¹H NMR (400 MHz, DMSO-d₆) δ ppm: 8.27 (d, J=5.6 Hz, 1H), 7.42-7.36 (m, 1H), 7.24-7.12 (m, 3H), 7.06 (t, J=8.0 Hz, 1H), 6.86 (d, J=5.7 Hz, 1H), 6.61-6.52 (m, 2H), 6.47-6.43 (m, 1H), 6.33 (s, 1H), 5.29-5.25 (m, 1H), 4.92-4.85 (m, 1H), 4.19-4.06 (m, 2H), 3.66 (s, 3H), 3.02 (dd, J=15.7, 7.1 Hz, 1H), 2.86-2.75 (m, 1H), 2.62-2.51 (m, 2H), 2.49-2.38 (m, 1H), 2.33-2.21 (m, 1H), 2.19-1.65 (m, 10H), 1.50-1.21 (m, 3H).

### Example 475 (1r,4r)-4-(3-chloroanilino)-2′-{3-[(7-hydroxy-6,7-dihydro-5H-cyclopenta[b]pyridin-4-yl)oxy]propyl}-2′,3′-dihydrospiro[cyclohexane-1,1′-indene]-4-carboxylic acid

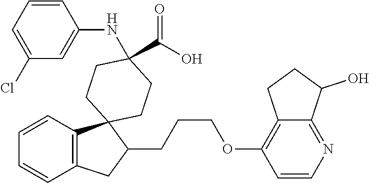

Using General procedure 33b and Example 475A (58 mg, 0.1 mmol, 1 eq) as the appropriate ester, a racemic mixture of diastereoisomers Example 475 was obtained as a white powder (31.6 mg, 0.06 mmol, 56%). LRMS calculated for C₃₂H₃₅ClN₂O₄: 546; found: 547 (M+H). ¹H NMR (400 MHz, DMSO-d₆) δ ppm: 8.27 (d, J=5.6 Hz, 1H), 7.42-7.34 (m, 1H), 7.24-7.12 (m, 3H), 7.05 (t, J=8.1 Hz, 1H), 6.86 (d, J=5.7 Hz, 1H), 6.64-6.60 (m, 1H), 6.58-6.51 (m, 2H), 6.23 (br s, 1H), 5.28 (br s, 1H), 4.93-4.85 (br m, 1H), 4.19-4.05 (m, 2H), 3.02 (dd, J=15.7, 7.2 Hz, 1H), 2.85-2.75 (m, 1H), 2.63-2.51 (m, 2H), 2.48-2.36 (m, 1H), 2.33-2.21 (m, 1H), 2.18-1.65 (m, 10H), 1.51-1.21 (m, 3H).

### Example 476

### Example 476A 4-chloro-7-fluoro-6,7-dihydro-5H-cyclopenta[b]pyridine

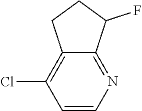 [SMILES: FC1CCc2c(Cl)ccnc21]

To a solution of 4-chloro-6,7-dihydro-5H-cyclopenta[b]pyridin-7-ol (500 mg, 2.95 mmol, 1 eq) in DCM (10 mL), cooled to −5° C., was added DAST (1.17 mL, 8.84 mmol, 3 eq). The mixture was slowly warmed to rt over 4 h. The mixture was cooled to 0° C., quenched by the dropwise addition of sat. aq. NH₄Cl solution and then basified to pH 11 with aq. NH₃ solution. The mixture was filtered through celite and the organic phase dried (PTFE phase separator) and concentrated in vacuo. Purification by automated flash chromatography (CombiFlash Rf, 12 g RediSep™ silica cartridge) eluting with a gradient of 0-40% EtOAc with 3% TEA in heptane afforded racemate Example 476A as a purple solid (358 mg, 2.09 mmol, 71%). LRMS calculated for C₈H₇ClFN: 171; found: 172 (M+H). ¹H NMR (400 MHz, DMSO-d₆) δ ppm: 8.50 (d, J=5.3 Hz, 1H), 7.56 (dd, J=5.3, 1.8 Hz, 1H), 6.00 (ddd, J=56.3, 6.7, 2.6 Hz, 1H), 3.17-3.06 (m, 1H), 3.00-2.87 (m, 1H), 2.61-2.44 (m, 1H), 2.32-2.16 (m, 1H).

### Example 476B methyl (1r,4r)-4-(3-chloroanilino)-2′-{3-[(7-fluoro-6,7-dihydro-5H-cyclopenta[b]pyridin-4-yl)oxy]propyl}-2′,3′-dihydrospiro[cyclohexane-1,1′-indene]-4-carboxylate

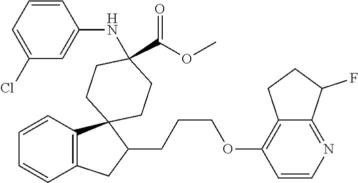

Using General procedure 31a and Preparation 9b (100 mg, 0.23 mmol, 1 eq) as the appropriate alcohol and Example 476A (48 mg, 0.28 mmol, 1.2 eq) as the appropriate aryl chloride, a racemic mixture of diastereoisomers Example 476B was obtained as a white foam (92 mg, 0.16 mmol, 70%). LRMS calculated for C₃₃H₃₆ClFN₂O₃: 562; found: 563 (M+H). ¹H NMR (400 MHz, DMSO-d₆) δ ppm: 8.37 (d, J=5.6 Hz, 1H), 7.42-7.36 (m, 1H), 7.25-7.12 (m, 3H), 7.09-6.99 (m, 2H), 6.61-6.53 (m, 2H), 6.47-6.42 (m, 1H), 6.33 (s, 1H), 5.97-5.77 (m, 1H), 4.24-4.09 (m, 2H), 3.66 (s, 3H), 3.02 (dd, J=15.7, 7.2 Hz, 1H), 2.96-2.84 (m, 1H), 2.81-2.68 (m, 1H), 2.62-2.53 (m, 1H), 2.49-2.29 (m, 2H), 2.22-1.66 (m, 10H), 1.50-1.22 (m, 3H).

### Example 476 (1r,4r)-4-(3-chloroanilino)-2′-{3-[(7-fluoro-6,7-dihydro-5H-cyclopenta[b]pyridin-4-yl)oxy]propyl}-2′,3′-dihydrospiro[cyclohexane-1,1′-indene]-4-carboxylic acid

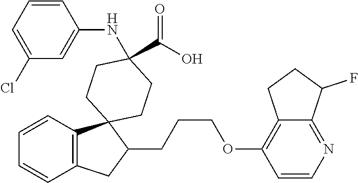

Using General procedure 33b and Example 476B (90 mg, 0.16 mmol, 1 eq) as the appropriate ester, a racemic mixture of diastereoisomers Example 476 was obtained as an off-white powder (35.2 mg, 0.06 mmol, 40%). LRMS calculated for C₃₂H₃₄ClFN₂O₃: 548; found: 549 (M+H). ¹H NMR (400 MHz, DMSO-d₆) δ ppm: 12.70 (br s, 1H), 8.37 (d, J=5.6 Hz, 1H), 7.42-7.35 (m, 1H), 7.24-7.12 (m, 3H), 7.09-6.99 (m, 2H), 6.62 (t, J=2.1 Hz, 1H), 6.58-6.51 (m, 2H), 6.24 (br s, 1H), 5.97-5.77 (m, 1H), 4.23-4.09 (m, 2H), 3.02 (dd, J=15.7, 7.2 Hz, 1H), 2.97-2.84 (m, 1H), 2.80-2.67 (m, 1H), 2.57 (dd, J=15.6, 7.9 Hz, 1H), 2.47-2.29 (m, 2H), 2.21-1.67 (m, 10H), 1.51-1.22 (m, 3H).

### Example 477 and Example 478

### Example 477A 5-methylidene-6,7-dihydro-5H-cyclopenta[b]pyridine

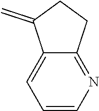

To an oven-dried flask was added methyltriphenylphosphonium bromide (3.49 g, 9.76 mmol, 1.3 eq) in THE (40 mL). The solution was cooled to 0° C. and nBuLi (4.1 mL, 2.38 M in hexanes, 9.76 mmol, 1.3 eq) was added dropwise. The solution was stirred at 0° C. for 40 min before the addition of 6,7-dihydro-5H-cyclopenta[b]pyridin-5-one (1 g, 7.51 mmol, 1 eq) in THE (10 mL). The solution was allowed to warm to rt and stirred for 18 h. The solution was cooled to 0° C., quenched with sat. aq. NaHCO₃ solution and the solvent was removed in vacuo. The mixture was extracted with EtOAc, and the combined organic extracts were washed with brine, dried (MgSO₄) and concentrated in vacuo. Purification by automated flash chromatography (CombiFlash Rf, 24 g RediSep™ silica cartridge) eluting with a gradient of 0-60% EtOAc with 3% TEA in heptane afforded Example 477A as a colorless oil (623 mg, 4.75 mmol, 63%). LRMS calculated for C₉H₉N: 131; found: 132 (M+H). ¹H NMR (400 MHz, DMSO-d₆) δ ppm: 8.40 (dd, J=4.8, 1.6 Hz, 1H), 7.94 (dd, J=7.8, 1.6 Hz, 1H), 7.24-7.20 (m, 1H), 5.63 (t, J=2.6 Hz, 1H), 5.15 (t, J=2.3 Hz, 1H), 3.02-2.97 (m, 2H), 2.80-2.74 (m, 2H).

### Example 477B 5-methyl-6,7-dihydro-5H-cyclopenta[b]pyridine

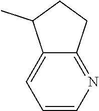

To a solution of Example 477A (623 mg, 4.75 mmol, 1 eq) in EtOH (20 mL) was added 10% Pd/C (50 mg) under a N₂ atmosphere. The mixture was evacuated and backfilled with N₂ (×3), then evacuated and backfilled with H₂ and shaken for 2 h at rt under an atmosphere of H₂. The reaction was filtered through celite, eluted with MeOH and evaporated under reduced pressure to afford racemate Example 477B as a colorless foam (624 mg, 4.69 mmol, 99%). LRMS calculated for C₉H₁₁N: 133; found: 134 (M+H). ¹H NMR (400 MHz, DMSO-d₆) δ ppm: 8.30-8.27 (m, 1H), 7.61-7.57 (m, 1H), 7.16-7.11 (m, 1H), 3.24-3.13 (m, 1H), 2.94-2.81 (m, 2H), 2.35-2.25 (m, 1H), 1.62-1.52 (m, 1H), 1.25 (d, J=6.9 Hz, 3H).

### Example 477C 5-methyl-1-oxo-6,7-dihydro-5H-1)⁵-cyclopenta[b]pyridine

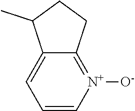

To a solution of Example 477B (620 mg, 4.65 mmol, 1 eq) in DCM (10 mL) was added mCPBA (1.12 g, 4.89 mmol, 1.05 eq) and the mixture was stirred at rt for 2.5 h. The mixture was loaded onto a MeOH-wet PE-AX cartridge (20 g), washed successively with 20% MeOH in DCM and MeOH, and concentrated in vacuo to afford racemate Example 477C as a yellow oil that was used directly in the subsequent step without further purification. LRMS calculated for C₉H₁₁NO: 149; found: 150 (M+H).

### Example 477D 4-chloro-5-methyl-6,7-dihydro-5H-cyclopenta[b]pyridine

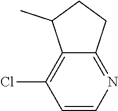

A solution of Example 477C (600 mg, 4.02 mmol, 1 eq) in POCl₃ (3.75 mL, 40.2 mmol, 10 eq) was heated at 90° C. for 48 h. The mixture was cooled to rt and added dropwise to a rapidly stirring solution of ice/water and stirred for 30 min. The mixture was basified with aq. NH₃ solution and then extracted with DCM. The organic phase was washed with brine, dried (MgSO₄) and concentrated in vacuo. Purification by automated flash chromatography (CombiFlash Rf, 12 g RediSep™ silica cartridge) eluting with a gradient of 0-47% EtOAc with 3% TEA in heptane afforded racemate Example 477D as an orange oil (380 mg, 2.27 mmol, 56%). LRMS calculated for C₉H₁₀ClN: 167; found: 168 (M+H). ¹H NMR (400 MHz, DMSO-d₆) δ ppm: 8.27 (dd, J=5.4, 0.6 Hz, 1H), 7.27-7.25 (m, 1H), 3.44-3.35 (m, 1H), 3.18-3.07 (m, 1H), 2.92-2.83 (m, 1H), 2.34-2.23 (m, 1H), 1.79-1.70 (m, 1H), 1.25 (d, J=7.0 Hz, 3H).

### Example 477 (1r,4r)-4-anilino-2′-{3-[(5-methyl-6,7-dihydro-5H-cyclopenta[b]pyridin-4-yl)oxy]propyl}-2′,3′-dihydrospiro[cyclohexane-1,1′-indene]-4-carboxylic acid

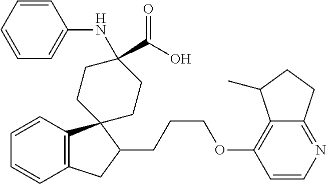

and

### Example 478 (1r,4r)-4-(3-chloroanilino)-2′-{3-[(5-methyl-6,7-dihydro-5H-cyclopenta[b]pyridin-4-yl)oxy]propyl}-2′,3′-dihydrospiro[cyclohexane-1,1′-indene]-4-carboxylic acid

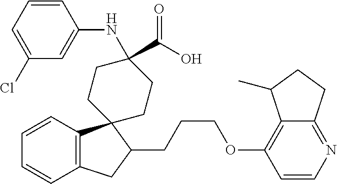

Using General procedure 31a and Preparation 9b (100 mg, 0.23 mmol, 1 eq) as the appropriate alcohol and Example 477D (47 mg, 0.28 mmol, 1.2 eq) as the appropriate aryl chloride and then further purification by SCX cartridge (2 g), washing successively with DCM and MeOH and eluting with 7 M NH₃ in MeOH/DCM (1:4), the desired intermediate was obtained. Using General procedure 33b, partial dehalogenation occurred. The products were separated by prep HPLC. The compound eluting earlier was collected as a racemic mixture of diastereoisomers Example 477 as a white powder (7.3 mg, 0.01 mmol, 6%). LRMS calculated for C₃₃H₃₈N₂O₃: 510; found: 511 (M+H). ¹H NMR (400 MHz, DMSO-d₆) δ ppm: 8.16 (d, J=5.7 Hz, 1H), 7.41-7.33 (m, 1H), 7.24-7.11 (m, 3H), 7.08-7.00 (m, 2H), 6.80-6.76 (m, 1H), 6.63-6.57 (m, 2H), 6.56-6.50 (m, 1H), 4.15-4.02 (m, 2H), 3.30-3.18 (m, 1H), 3.07-2.98 (m, 1H), 2.97-2.87 (m, 1H), 2.80-2.68 (m, 1H), 2.57 (dd, J=15.6, 7.5 Hz, 1H), 2.47-2.37 (m, 1H), 2.25-1.64 (m, 10H), 1.62-1.51 (m, 1H), 1.50-1.21 (m, 3H), 1.17/1.14 (d, J=6.9 Hz, 3H).

The compound eluting later was collected as a racemic mixture of diastereoisomers, Example 478 as a white powder (24 mg, 0.04 mmol, 19%). LRMS calculated for C₃₃H₃₇ClN₂O₃: 544; found: 545 (M+H). ¹H NMR (400 MHz, DMSO-d₆) δ ppm: 12.72 (br s, 1H), 8.16 (d, J=5.7 Hz, 1H), 7.41-7.34 (m, 1H), 7.24-7.11 (m, 3H), 7.09-7.01 (m, 1H), 6.80-6.76 (m, 1H), 6.65-6.60 (m, 1H), 6.58-6.50 (m, 2H), 6.20 (br s, 1H), 4.15-4.03 (m, 2H), 3.29-3.17 (m, 1H), 3.08-2.99 (m, 1H), 2.97-2.87 (m, 1H), 2.80-2.68 (m, 1H), 2.57 (dd, J=15.6, 7.7 Hz, 1H), 2.48-2.37 (m, 1H), 2.25-1.95 (m, 4H), 1.94-1.64 (m, 6H), 1.61-1.51 (m, 1H), 1.51-1.21 (m, 3H), 1.16/1.14 (d, J=6.9 Hz, 3H).

### Example 479 (1r,4r)-4-(3-chloroanilino)-2′-{3-[(1-methyl-2-oxo-1,2-dihydropyridin-4-yl)oxy]propyl}-2′,3′-dihydrospiro[cyclohexane-1,1′-indene]-4-carboxylic acid

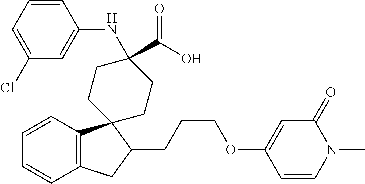

Using General procedure 31a and Preparation 9b (50 mg, 0.12 mmol, 1 eq) as the appropriate alcohol and 4-bromo-1-methylpyridin-2 (1H)-one (26 mg, 0.14 mmol, 1.2 eq) as the appropriate aryl chloride, the desired intermediate was obtained. The material was stirred in MeOH (3 mL) and 3 M aq. NaOH solution (0.1 mL) was added. The mixture was heated at 110° C. for 1 h under microwave irradiation. The mixture was concentrated in vacuo and suspended in a minimum amount of water, acidified with 2 M aq. HCl solution and the organics were extracted with DCM. The organic phase was washed with brine, dried (MgSO₄) and concentrated in vacuo. Purification by preparative HPLC at pH 4 afforded racemate Example 479 as an off-white solid (1.62 mg, 3.11 μmol, 3%). LRMS calculated for C₃₀H₃₃ClN₂O₄: 520; found: 521 (M+H). ¹H NMR (400 MHz, DMSO-d₆) δ ppm: 7.53 (d, J=7.5 Hz, 1H), 7.41-7.34 (m, 1H), 7.25-7.12 (m, 3H), 7.05 (t, J=8.1 Hz, 1H), 6.62 (t, J=2.2 Hz, 1H), 6.58-6.52 (m, 2H), 6.19 (br s, 1H), 5.90 (dd, J=7.5, 2.7 Hz, 1H), 5.78 (d, J=2.8 Hz, 1H), 3.96 (t, J=6.2 Hz, 2H), 3.32 (s, 3H), 3.00 (dd, J=15.6, 7.2 Hz, 1H), 2.56 (dd, J=15.6, 7.7 Hz, 1H), 2.47-2.37 (m, 1H), 2.18-1.57 (m, 9H), 1.51-1.33 (m, 2H), 1.32-1.18 (m, 1H).

### Example 480

### Example 480A methyl (1r,4r)-4-(3-chloroanilino)-2′-{3-[(1-methyl-2-oxo-2,3-dihydro-1H-indol-5-yl)oxy]propyl}-2′,3′-dihydrospiro[cyclohexane-1,1′-indene]-4-carboxylate

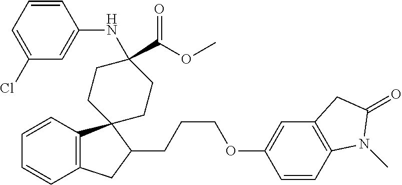

Using General procedure 30b and Preparation 9b (200 mg, 0.47 mmol, 1 eq) as the appropriate indane and 5-hydroxy-1-methyl-1,3-dihydro-2H-indol-2-one (92 mg, 0.56 mmol, 1.2 eq) as the appropriate alcohol, racemate Example 480A was obtained as a white foam (119 mg, 0.21 mmol, 44%). LRMS calculated for C₃₄H₃₇ClN₂O₄: 572; found: 573 (M+H). ¹H NMR (400 MHz, DMSO-d₆) δ ppm: 7.41-7.35 (m, 1H), 7.24-7.12 (m, 3H), 7.07 (t, J=8.0 Hz, 1H), 6.94-6.91 (m, 1H), 6.86-6.83 (m, 2H), 6.62-6.55 (m, 2H), 6.48-6.43 (m, 1H), 6.33 (s, 1H), 3.96 (t, J=6.0 Hz, 2H), 3.66 (s, 3H), 3.49 (d, J=1.1 Hz, 2H), 3.08 (s, 3H), 3.01 (dd, J=15.7, 7.1 Hz, 1H), 2.57 (dd, J=15.7, 7.7 Hz, 1H), 2.49-2.39 (m, 1H), 2.19-1.62 (m, 9H), 1.50-1.34 (m, 2H), 1.33-1.22 (m, 1H).

### Example 480 (1r,4r)-4-(3-chloroanilino)-2′-{3-[(1-methyl-2-oxo-2,3-dihydro-1H-indol-5-yl)oxy]propyl}-2′,3′-dihydrospiro[cyclohexane-1,1′-indene]-4-carboxylic acid

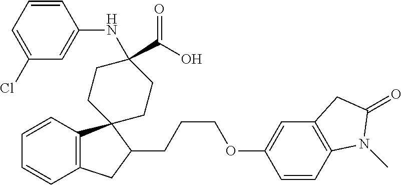

Using General procedure 33b and Example 480A (119 mg, 0.21 mmol, 1 eq) as the appropriate ester, racemate Example 480 was obtained as an off-white powder (12.9 mg, 0.02 mmol, 11%). LRMS calculated for C₃₃H₃₅ClN₂O₄: 558; found: 559 (M+H). ¹H NMR (400 MHz, DMSO-d₆) δ ppm: 12.72 (br s, 1H), 7.41-7.34 (m, 1H), 7.25-7.10 (m, 3H), 7.06 (t, J=8.1 Hz, 1H), 6.94-6.91 (m, 1H), 6.86-6.81 (m, 2H), 6.63 (t, J=2.1 Hz, 1H), 6.57-6.52 (m, 2H), 6.22 (br s, 1H), 3.96 (t, J=6.1 Hz, 2H), 3.49 (d, J=1.1 Hz, 2H), 3.08 (s, 3H), 3.01 (dd, J=15.7, 7.2 Hz, 1H), 2.57 (dd, J=15.7, 7.7 Hz, 1H), 2.48-2.37 (m, 1H), 2.18-1.62 (m, 9H), 1.51-1.36 (m, 2H), 1.33-1.21 (m, 1H).

### Example 481 (1r,2′R,4R)-4-(3-chloroanilino)-2′-{3-[(2-methyl-1H-indol-4-yl)oxy]propyl}-2′,3′-dihydrospiro[cyclohexane-1,1′-indene]-4-carboxylic acid

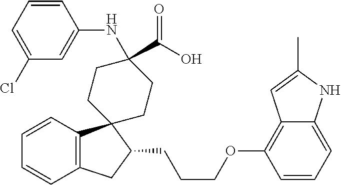 [SMILES: Cc1cc2c(OCCC[C@@H]3Cc4ccccc4[C@]34CC[C@@](Nc3cccc(Cl)c3)(C(=O)O)CC4)cccc2[nH]1]

and

### Example 482 (1r,2′S,4S)-4-(3-chloroanilino)-2′-{3-[(2-methyl-1H-indol-4-yl)oxy]propyl}-2′,3′-dihydrospiro[cyclohexane-1,1′-indene]-4-carboxylic acid

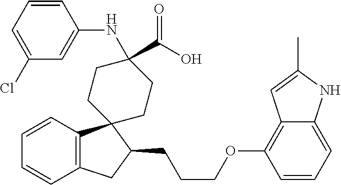 [SMILES: Cc1cc2c(OCCC[C@H]3Cc4ccccc4[C@]34CC[C@@](Nc3cccc(Cl)c3)(C(=O)O)CC4)cccc2[nH]1]

Using General procedure 32 and Preparation 9b1 as the appropriate indane and 2-methyl-1H-indol-4-ol as the appropriate alcohol Example 481 was obtained. ¹H NMR (500 MHz, DMSO-d₆) δ ppm: 10.83 (s, 1H), 7.37 (dm, 1H), 7.21 (dm, 1H), 7.18-7.10 (m, 2H), 7.00 (t, 1H), 6.85 (m, 2H), 6.61 (t, 1H), 6.55 (dm, 1H), 6.50 (dm, 1H), 6.42 (m, 1H), 6.17 (br, 1H), 6.08 (br s, 1H), 4.06 (m, 2H), 3.04/2.59 (dd+dd, 2H), 2.46-1.25 (m, 12H), 2.31 (s, 3H), 2.07 (m, 1H). HRMS calculated for C₃₃H₃₅N₂O₃Cl: 542.2336; found: 543.2414 (M+H).

Using General procedure 32 and Preparation 9b2 as the appropriate indane and 2-methyl-1H-indol-4-ol as the appropriate alcohol Example 482 was obtained. HRMS calculated for C₃₃H₃₅N₂O₃Cl: 542.2336; found: 543.2411 (M+H).

### Example 483 (1r,2′R,4R)-4-(3-chloroanilino)-2′-{3-[(3-methyl-1H-indol-4-yl)oxy]propyl}-2′,3′-dihydrospiro[cyclohexane-1,1′-indene]-4-carboxylic acid

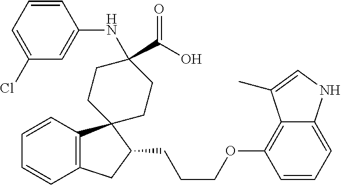 [SMILES: Cc1c[nH]c2cccc(OCCC[C@@H]3Cc4ccccc4[C@]34CC[C@@](Nc3cccc(Cl)c3)(C(=O)O)CC4)c12]

and

### Example 484 (1r,2′S,4S)-4-(3-chloroanilino)-2′-{3-[(3-methyl-1H-indol-4-yl)oxy]propyl}-2′,3′-dihydrospiro[cyclohexane-1,1′-indene]-4-carboxylic acid

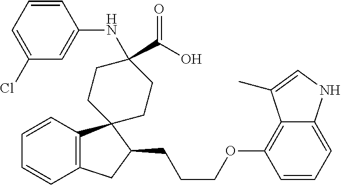 [SMILES: Cc1c[nH]c2cccc(OCCC[C@H]3Cc4ccccc4[C@]34CC[C@@](Nc3cccc(Cl)c3)(C(=O)O)CC4)c12]

Using General procedure 32 and Preparation 9b1 as the appropriate indane and 3-methyl-1H-indol-4-ol as the appropriate alcohol Example 483 was obtained. ¹H NMR (500 MHz, DMSO-d₆) δ ppm: 10.62 (d, 1H), 7.40-7.10 (m, 4H), 6.99 (t, 1H), 6.88 (t, 1H), 6.86 (m, 1H), 6.84 (d, 1H), 6.63 (t, 1H), 6.53 (dd, 1H), 6.48 (dd, 1H), 6.36 (dd, 1H), 6.08 (br s, 1H), 4.03/4.01 (m+m, 2H), 3.05/2.59 (dd+dd, 2H), 2.47-1.30 (m, 12H), 2.30 (d, 3H), 2.07 (m, 1H). HRMS calculated for C₃₃H₃₅N₂O₃Cl: 542.2336; found: 543.2408 (M+H).

Using General procedure 32 and Preparation 9b2 as the appropriate indane and 3-methyl-1H-indol-4-ol as the appropriate alcohol Example 484 was obtained. HRMS calculated for C₃₃H₃₅N₂O₃Cl: 542.2336; found: 543.2407 (M+H).

### Example 485 (1r,2′R,4R)-4-(3-chloroanilino)-2′-{3-[(1H-indol-4-yl)oxy]propyl}-2′,3′-dihydrospiro[cyclohexane-1,1′-indene]-4-carboxylic acid

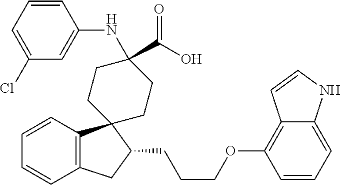 [SMILES: O=C(O)[C@]1(Nc2cccc(Cl)c2)CC[C@@]2(CC1)c1ccccc1C[C@H]2CCCOc1cccc2[nH]ccc12]

and

### Example 486 (1r,2′S,4S)-4-(3-chloroanilino)-2′-{3-[(1H-indol-4-yl)oxy]propyl}-2′,3′-dihydrospiro[cyclohexane-1,1′-indene]-4-carboxylic acid

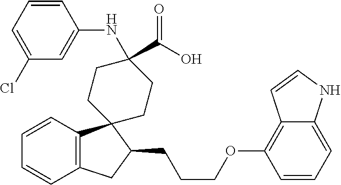 [SMILES: O=C(O)[C@]1(Nc2cccc(Cl)c2)CC[C@@]2(CC1)c1ccccc1C[C@@H]2CCCOc1cccc2[nH]ccc12]

Using General procedure 32 and Preparation 9b1 as the appropriate indane and 1H-indol-4-ol as the appropriate alcohol Example 485 was obtained. HRMS calculated for C₃₂H₃₃N₂O₃Cl: 528.2180; found: 529.2264 (M+H).

Using General procedure 32 and Preparation 9b2 as the appropriate indane and 1H-indol-4-ol as the appropriate alcohol Example 486 was obtained. HRMS calculated for C₃₂H₃₃N₂O₃Cl: 528.2180; found: 529.2240 (M+H).

### Example 487 (1r,2′R,4R)-4-(3-chloroanilino)-2′-{3-[(6-methyl-1H-indol-4-yl)oxy]propyl}-2′,3′-dihydrospiro[cyclohexane-1,1′-indene]-4-carboxylic acid

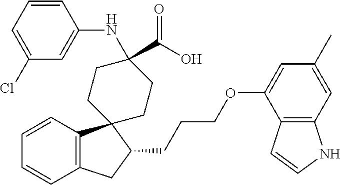 [SMILES: Cc1cc(OCCC[C@@H]2Cc3ccccc3[C@]23CC[C@@](Nc2cccc(Cl)c2)(C(=O)O)CC3)c2cc[nH]c2c1]

and

### Example 488 (1r,2′S,4S)-4-(3-chloroanilino)-2′-{3-[(6-methyl-1H-indol-4-yl)oxy]propyl}-2′,3′-dihydrospiro[cyclohexane-1,1′-indene]-4-carboxylic acid

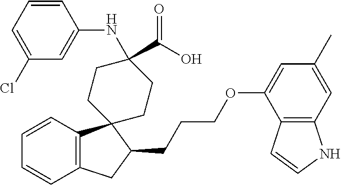 [SMILES: Cc1cc(OCCC[C@H]2Cc3ccccc3[C@]23CC[C@@](Nc2cccc(Cl)c2)(C(=O)O)CC3)c2cc[nH]c2c1]

Using General procedure 32 and Preparation 9b1 as the appropriate indane and 6-methyl-1H-indol-4-ol as the appropriate alcohol Example 487 was obtained. ¹H NMR (500 MHz, DMSO-d₆) δ ppm: 12.65 (br s, 1H), 10.85 (br s, 1H), 7.40-7.10 (m, 4H), 7.05 (dd, 1H), 7.04 (t, 1H), 6.75 (br s, 1H), 6.60 (t, 1H), 6.54 (dm, 1H), 6.54 (dm, 1H), 6.31 (dd, 1H), 6.30 (br s, 1H), 6.22 (br s, 1H), 4.08/4.05 (m+m, 2H), 3.05/2.6 (dd+dd, 2H), 2.47-1.26 (m, 12H), 2.34 (s, 3H), 2.07 (m, 1H). HRMS calculated for C₃₃H₃₅N₂O₃Cl: 542.2336; found: 543.2406 (M+H).

Using General procedure 32 and Preparation 9b2 as the appropriate indane and 6-methyl-1H-indol-4-ol as the appropriate alcohol Example 488 was obtained. HRMS calculated for C₃₃H₃₅N₂O₃Cl: 542.2336; found: 543.2403 (M+H).

### Example 489 (1r,2′R,4R)-4-(3-chloroanilino)-2′-{3-[(5-methyl-1H-indol-4-yl)oxy]propyl}-2′,3′-dihydrospiro[cyclohexane-1,1′-indene]-4-carboxylic acid

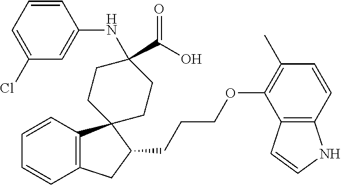 [SMILES: Cc1ccc2[nH]ccc2c1OCCC[C@@H]1Cc2ccccc2[C@]12CC[C@@](Nc1cccc(Cl)c1)(C(=O)O)CC2]

and

### Example 490 (1r,2′S,4S)-4-(3-chloroanilino)-2′-{3-[(5-methyl-1H-indol-4-yl)oxy]propyl}-2′,3′-dihydrospiro[cyclohexane-1,1′-indene]-4-carboxylic acid

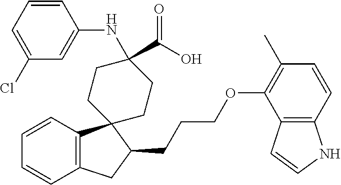 [SMILES: Cc1ccc2[nH]ccc2c1OCCC[C@H]1Cc2ccccc2[C@]12CC[C@@](Nc1cccc(Cl)c1)(C(=O)O)CC2]

Using General procedure 32 and Preparation 9b1 as the appropriate indane and 5-methyl-1H-indol-4-ol as the appropriate alcohol Example 489 was obtained. ¹H NMR (500 MHz, DMSO-d₆) δ ppm: 10.99 (br s, 1H), 7.42-7.10 (m, 4H), 7.19 (dd, 1H), 7.02 (t, 1H), 7.00 (dd, 1H), 6.84 (d, 1H), 6.63 (t, 1H), 6.54 (dm, 1H), 6.51 (dm, 1H), 6.40 (m, 1H), 6.18 (br s, 1H), 4.12/4.06 (m+m, 2H), 3.01/2.58 (dd+dd, 2H), 2.47-1.28 (m, 12H), 2.22 (s, 3H), 2.05 (m, 1H). HRMS calculated for C₃₃H₃₅N₂O₃Cl: 542.2336; found: 543.2395 (M+H).

Using General procedure 32 and Preparation 9b2 as the appropriate indane and 5-methyl-1H-indol-4-ol as the appropriate alcohol Example 490 was obtained. HRMS calculated for C₃₃H₃₅N₂O₃Cl: 542.2336; found: 543.2403 (M+H).

### Example 491 (1r,4r)-4-(3-chloroanilino)-2′-{2-(hydroxymethyl)-3-[(thieno[3,2-b]pyridin-7-yl)oxy]propyl}spiro[cyclohexane-1,1′-indene]-4-carboxylic acid

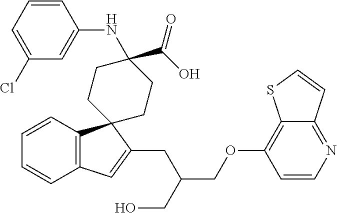 [SMILES: O=C(O)[C@]1(Nc2cccc(Cl)c2)CC[C@]2(CC1)C(CC(CO)COc1ccnc3ccsc13)=Cc1ccccc12]

and

### Example 492 (1r,4r)-4-(3-chloroanilino)-2′-(3-[(thieno[3,2-b]pyridin-7-yl)oxy]-2-{[(thieno[3,2-b]pyridin-7-yl)oxy]methyl}propyl)spiro[cyclohexane-1,1′-indene]-4-carboxylic acid

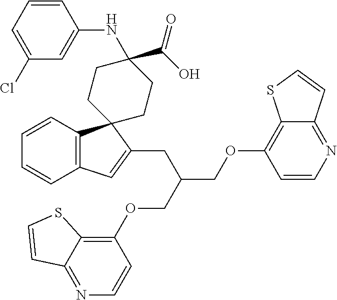 [SMILES: O=C(O)[C@]1(Nc2cccc(Cl)c2)CC[C@]2(CC1)C(CC(COc1ccnc3ccsc13)COc1ccnc3ccsc13)=Cc1ccccc12]

To a solution of Preparation 11a (63 mg, 0.14 mmol, 1 eq) in DMF (3 mL), cooled to 0° C., was added thieno[3,2-b]pyridine-7-ol (25 mg, 0.17 mmol, 1.2 eq) and PPh₃ (217 mg, 0.85 mmol, 6 eq) followed by DIAD (163 μL, 0.85 mmol, 6 eq) dropwise and the mixture was stirred at rt for 18 h. The reaction was partitioned between DCM and water, and the organic phase was washed with brine, dried (MgSO₄) and concentrated in vacuo. Purification by automated flash column chromatography (CombiFlash Rf, 40 g RediSep™ silica cartridge) eluting with a gradient of 0-100% EtOAc in heptane afforded two intermediates. The intermediate eluting earlier was hydrolyzed according to General procedure 33b to obtain Example 491 as a racemate, isolated as a beige solid (9.62 mg, 0.02 mmol, 12%). LRMS calculated for C₃₂H₃₁ClN₂O₄S: 574; found: 575 (M+H). ¹H NMR (400 MHz, DMSO-d₆) δ ppm: 12.78 (br s, 1H), 8.53 (d, J=5.5 Hz, 1H), 7.93 (d, J=5.4 Hz, 1H), 7.68-7.64 (m, 1H), 7.50 (d, J=5.4 Hz, 1H), 7.32-7.27 (m, 1H), 7.26-7.19 (m, 1H), 7.15-7.07 (m, 2H), 7.04 (d, J=5.5 Hz, 1H), 6.64 (t, J=2.1 Hz, 1H), 6.63-6.55 (m, 3H), 6.38 (br s, 1H), 4.92-4.73 (br m, 1H), 4.41-4.29 (m, 2H), 3.68-3.55 (m, 2H), 2.50-2.32 (m, 5H), 2.28-2.08 (m, 4H), 0.98-0.88 (m, 1H), 0.83-0.74 (m, 1H). The intermediate eluting later was hydrolyzed according to General procedure 33b to obtain Example 492 as a beige solid (8.18 mg, 0.01 mmol, 8%). LRMS calculated for C₃₉H₃₄ClN₃O₄S₂: 707; found: 708 (M+H). ¹H NMR (400 MHz, DMSO-d₆) δ ppm: 12.72 (br s, 1H), 8.48 (d, J=5.5 Hz, 2H), 7.89 (d, J=5.4 Hz, 2H), 7.66 (d, J=7.5 Hz, 1H), 7.48 (d, J=5.4 Hz, 2H), 7.32-7.27 (m, 1H), 7.26-7.19 (m, 1H), 7.16-7.08 (m, 2H), 7.03 (d, J=5.5 Hz, 2H), 6.74 (s, 1H), 6.67-6.55 (m, 3H), 6.39 (br s, 1H), 4.57-4.49 (m, 2H), 4.49-4.41 (m, 2H), 3.09-2.98 (m, 1H), 2.70-2.62 (m, 2H), 2.46-2.32 (m, 2H), 2.30-2.10 (m, 4H), 0.92-0.81 (m, 2H).

### Example 493 (1r,4r)-4-(3-chloroanilino)-2′-[3-methoxy-2-(methoxymethyl)propyl]spiro[cyclohexane-1,1′-indene]-4-carboxylic acid

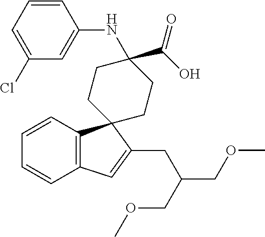 [SMILES: COCC(COC)CC1=Cc2ccccc2[C@]12CC[C@@](Nc1cccc(Cl)c1)(C(=O)O)CC2]

To a solution of Preparation 11a (41 mg, 0.09 mmol, 1 eq) in DMF (3 mL), cooled to 0° C., was added NaH (60% dispersion; 9 mg, 0.22 mmol, 2.5 eq) and the mixture was stirred for 30 min. Mel (14 μL, 0.22 mmol, 2.5 eq) was added, and the mixture was allowed to warm to rt and stirred for 18 h. The reaction was concentrated in vacuo, partitioned between EtOAc and water, and the organic phase was washed with brine, dried (MgSO₄) and concentrated in vacuo. Purification by automated flash column chromatography (CombiFlash Rf, 12 g RediSep™ silica cartridge) eluting with a gradient of 0-20% MeOH in DCM afforded an intermediate, which was hydrolyzed according to General procedure 33b to obtain Example 493 as a white solid (9.09 mg, 0.02 mmol, 21%). LRMS calculated for C₂₇H₃₂ClNO₄: 469; found: 470 (M+H). ¹H NMR (400 MHz, DMSO-d₆) δ ppm: 7.67 (d, J=7.5 Hz, 1H), 7.30 (dd, J=7.5, 1.2 Hz, 1H), 7.23 (td, J=7.4, 0.9 Hz, 1H), 7.15-7.07 (m, 2H), 6.65 (t, J=2.1 Hz, 1H), 6.60-6.54 (m, 2H), 6.51 (s, 1H), 6.34 (br s, 1H), 3.41-3.29 (m, 4H), 3.22 (s, 6H), 2.47-2.35 (m, 2H), 2.29-2.04 (m, 7H), 0.92-0.81 (m, 2H).

### Example 494 (1r,4r)-4-(3-chloroanilino)-2′-(2-{[(6,7-dihydro-5H-cyclopenta[b]pyridin-4-yl)oxy]methyl}-3-methoxypropyl)-2′,3′-dihydrospiro[cyclohexane-1,1′-indene]-4-carboxylic acid

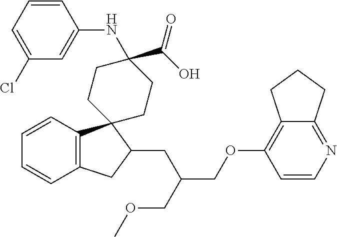

To a solution of Preparation 11b (102 mg, 0.22 mmol, 1 eq) in DMF (2 mL), cooled to 0° C., was added NaH (60% dispersion; 11 mg, 0.27 mmol, 1.2 eq) portionwise. The mixture was stirred at rt for 30 min. Mel (17 μL, 0.27 mmol, 1.2 eq) was added and the mixture was stirred at rt for 72 h. The mixture was quenched with water and extracted with EtOAc, and the combined organic extracts were washed successively with water and brine, dried (MgSO₄) and concentrated in vacuo. Purification by automated flash column chromatography (CombiFlash Rf, 24 g RediSep™ silica cartridge) eluting with a gradient of 0-18% MeOH in DCM afforded a solid that was taken up in DMSO (2 mL) and to this was added KOtBu (39 mg, 0.34 mmol, 1.5 eq), followed by 4-chloro-6,7-dihydro-5H-cyclopenta[b]pyridine (35 mg, 0.23 mmol, 1.05 eq) and the mixture was heated at 50° C. for 18 h. After cooling, it was partitioned between EtOAc and 0.2 M aq. HCl solution. The phases were separated, and the aq. phase was extracted with EtOAc. The combined organic extracts were washed with brine, dried (MgSO₄) and concentrated in vacuo. Purification by preparative HPLC at pH4 afforded a racemic mixture of diastereoisomers, Example 494 as a beige solid (13 mg, 0.022 mmol, 10%). LRMS calculated for C₃₄H₃₉ClN₂O₄: 574; found: 575 (M+H). ¹H NMR (400 MHz, DMSO-d₆) δ ppm: 8.19-8.12 (m, 1H), 7.43-7.31 (m, 1H), 7.25-7.08 (m, 3H), 7.07-6.98 (m, 1H), 6.82-6.74 (m, 1H), 6.68-6.60 (m, 1H), 6.60-6.45 (m, 2H), 6.07 (br s, 1H), 4.13-3.94 (m, 2H), 3.50-3.31 (m, 2H), 3.26/3.25 (s, 3H), 3.08-2.93 (m, 1H), 2.90-2.66 (m, 4H), 2.63-2.31 (m, 2H), 2.22-1.12 (m, 13H).

### Example 495 and Example 496 and Example 497 and Example 498

### Example 495A (1r,4r)-4-(3-chloroanilino)-2′-{2-(hydroxymethyl)-3-[2-(morpholin-4-yl)ethoxy]propyl}-2′,3′-dihydrospiro[cyclohexane-1,1′-indene]-4-carboxylic acid

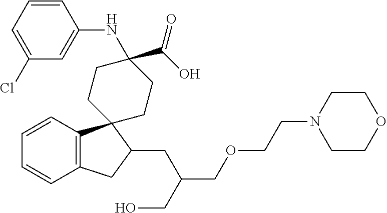

To a solution of Preparation 11b (200 mg, 0.44 mmol, 1 eq) in DMF (4 mL) at 0° C. was added NaH (60% dispersion; 21 mg, 0.52 mmol, 1.2 eq) portionwise and the mixture was stirred for further 30 min at 0° C. before 4-(2-chloroethyl)morpholine×HCl (98 mg, 0.52 mmol, 1.2 eq) was added, then the mixture was allowed to warm to rt and stirred for 18 h. The reaction was cooled to 0° C. and NaH (60% dispersion; 21 mg, 0.52 mmol, 1.2 eq) was added portionwise, stirred for further 30 min at 0° C., before 4-(2-chloroethyl)morpholine×HCl (98 mg, 0.52 mmol, 1.2 eq) was added and it was stirred for 48 h at rt. The mixture was partitioned between EtOAc and water. The phases were separated, and the aq. phase was acidified to pH 5 using 1 M aq. HCl solution and extracted with EtOAc. The combined organic extracts were washed with brine, dried (MgSO₄) and concentrated in vacuo to give a racemic mixture of diastereoisomers, Example 495A, isolated as a solid (103 mg, 0.18 mmol, 42%). ¹H NMR (400 MHz, DMSO-d₆) δ ppm: 7.39-7.31 (m, 1H), 7.24-7.10 (m, 3H), 7.06 (t, J=8.1 Hz, 1H), 6.65-6.61 (m, 1H), 6.58-6.52 (m, 2H), 6.20 (br s, 1H), 4.39-4.31 (m, 1H), 3.54-3.23 (m, 10H), 3.02-2.93 (m, 1H), 2.59-2.29 (m, 8H), 2.21-2.06 (m, 2H), 2.02-1.50 (m, 5H), 1.50-1.10 (m, 4H).

### Example 495 and Example 496 (1r,4r)-4-(3-chloroanilino)-2′-(2-{[(6,7-dihydro-5H-cyclopenta[b]pyridin-4-yl)oxy]methyl}-3-[2-(morpholin-4-yl)ethoxy]propyl)-2′,3′-dihydrospiro[cyclohexane-1,1′-indene]-4-carboxylic acid

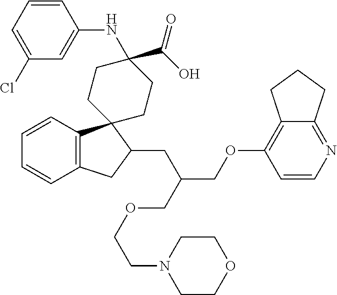

and

### Example 497 (1r,4r)-4-(3-chloroanilino)-2′-(3-[(6,7-dihydro-5H-cyclopenta[b]pyridin-4-yl)oxy]-2-{[(6,7-dihydro-5H-cyclopenta[b]pyridin-4-yl)oxy]methyl}propyl)-2′,3′-dihydrospiro[cyclohexane-1,1′-indene]-4-carboxylic acid, enantiomer 1

and

### Example 498 (1r,4r)-4-(3-chloroanilino)-2′-(3-[(6,7-dihydro-5H-cyclopenta[b]pyridin-4-yl)oxy]-2-{[(6,7-dihydro-5H-cyclopenta[b]pyridin-4-yl)oxy]methyl}propyl)-2′,3′-dihydrospiro[cyclohexane-1,1′-indene]-4-carboxylic acid, enantiomer 2

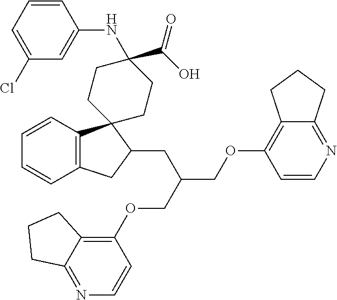

To a solution of Example 495A (103 mg, 0.18 mmol, 1 eq) in DMSO (3 mL) was added KOtBu (62 mg, 0.55 mmol, 3 eq), followed by 4-chloro-6,7-dihydro-5H-cyclopenta[b]pyridine (57 mg, 0.37 mmol, 2 eq) and the mixture was heated at 50° C. for 18 h. The mixture was allowed to cool to rt and loaded onto a MeOH-wet PE-AX cartridge (10 g), washed successively with MeOH and DCM, eluting with 10% HCOOH in DCM, and concentrated in vacuo. The two diastereoisomers were partially separated using reverse phase automated flash chromatography (CombiFlash Rf, C18 15.5 g Gold RediSep column) eluting with a gradient of 20-100% MeCN in water. The mixture eluting earlier was collected as Example 495 (contained 2 diastereoisomer pairs in 87:13 ratio) and was isolated as an off-white solid (13.1 mg, 0.02 mmol, 11%). LRMS calculated for C₃₉H₄ClN₃O₅: 673; found: 674 (M+H). ¹H NMR (400 MHz, DMSO-d₆) δ ppm: 8.15 (d, J=5.7 Hz, 1H), 7.41-7.32 (m, 1H), 7.24-7.11 (m, 3H), 7.03 (t, J=8.1 Hz, 1H), 6.78 (d, J=5.7 Hz, 1H), 6.63 (t, J=2.0 Hz, 1H), 6.56-6.50 (m, 2H), 6.17 (br s, 1H), 4.16-4.02 (m, 2H), 3.55-3.38 (m, 8H), 3.07-2.97 (m, 1H), 2.85-2.74 (m, 2H), 2.74-2.67 (m, 2H), 2.62-2.53 (m, 1H), 2.47-2.25 (m, 7H), 2.21-2.06 (m, 3H), 2.04-1.70 (m, 6H), 1.68-1.57 (m, 1H), 1.51-1.21 (m, 3H).

The mixture eluting later was collected as Example 496 (contained 2 diastereoisomer pairs in 14:86 ratio) and was isolated as an off-white solid (13.8 mg, 0.02 mmol, 11%). LRMS calculated for C₃₉H₄₈ClN₃O₅: 673; found: 674 (M+H). ¹H NMR (400 MHz, DMSO-d₆) δ ppm: 8.16 (d, J=5.7 Hz, 1H), 7.41-7.32 (m, 1H), 7.23-7.11 (m, 3H), 7.04 (t, J=8.1 Hz, 1H), 6.77 (d, J=5.7 Hz, 1H), 6.65 (t, J=2.0 Hz, 1H), 6.58-6.52 (m, 2H), 6.22 (br s, 1H), 4.08-3.96 (m, 2H), 3.57-3.38 (m, 8H), 3.05-2.96 (m, 1H), 2.90-2.71 (m, 4H), 2.59-2.51 (m, 1H), 2.48-2.39 (m, 3H), 2.33-2.25 (m, 4H), 2.22-1.69 (m, 9H), 1.64-1.54 (m, 1H), 1.50-1.22 (m, 3H).

An intermediate eluting last was also isolated. It was used as the appropriate carboxylic acid and treated as described in General Procedure 17a to obtain a racemate. The enantiomers were separated by chiral chromatography. Column: AD, 50×500 mm, 20 μm. Eluents: 50:50 nPrOH/Heptane. The enantiomer eluting earlier was hydrolyzed according to General procedure 33a to obtain Example 497. ¹H NMR (500 MHz, DMSO-d₆) δ ppm: 12.71 (br s, 1H), 8.16/8.15 (d, 2H), 7.42-7.1 (m, 4H), 7.03 (t, 1H), 6.81/6.8 (d, 2H), 6.62 (t, 1H), 6.53 (dm, 1H), 6.51 (dm, 1H), 6.16 (br s, 1H), 4.21/4.17/4.16 (dd+dd/d, 4H), 3.03/2.59 (dd+dd, 2H), 2.89-1.78 (m, 12H), 2.46-1.33 (m, 8H), 2.37 (m, 1H), 2.19 (m, 1H), 1.77/1.42 (m+m, 2H). HRMS calculated for C₄₁H₄₄N₃O₄Cl: 677.3021; found: 678.3084 (M+H).

The enantiomer eluting later was hydrolyzed according to General procedure 33a to obtain Example 498. HRMS calculated for C₄₁H₄₄N₃O₄Cl: 677.3021; found: 678.3081 (M+H).

### Example 499 (1r,4r)-4-(3-chloroanilino)-2′-(2-{[(6,7-dihydro-5H-cyclopenta[b]pyridin-4-yl)oxy]methyl}-3-hydroxypropyl)-2′,3′-dihydrospiro[cyclohexane-1,1′-indene]-4-carboxylic acid, diastereoisomer 1

and

### Example 500 (1r,4r)-4-(3-chloroanilino)-2′-(2-{[(6,7-dihydro-5H-cyclopenta[b]pyridin-4-yl)oxy]methyl}-3-hydroxypropyl)-2′,3′-dihydrospiro[cyclohexane-1,1′-indene]-4-carboxylic acid, diastereoisomer 2

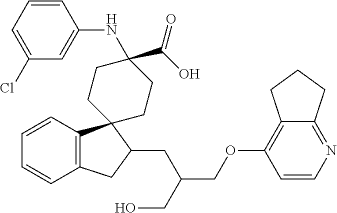

To a solution of Preparation 11b (311 mg, 0.68 mmol, 1 eq) in DMSO (5 mL) was added KOtBu (229 mg, 2.04 mmol, 3 eq), followed by 4-chloro-6,7-dihydro-5H-cyclopenta[b]pyridine (172 mg, 1.12 mmol, 1.65 eq) and the mixture was heated at 50° C. for 18 h. After cooling to rt, the mixture was partitioned between DCM and 0.2 M aq. HCl solution. The phases were separated, and the aq. phase was extracted with DCM. The combined organic extracts were washed with water, brine, dried (MgSO₄) and concentrated in vacuo to afford a racemic mixture of diastereoisomers. They were partially separated via reverse phase automated flash chromatography (CombiFlash Rf, C18 26 g RediSep column) eluting with a gradient of 5-100% MeCN in water. The diastereoisomer pair eluting earlier was collected as Example 499 and was isolated as a white solid (21.9 mg, 0.04 mmol, 6%). LRMS calculated for C₃₃H₃₇ClN₂O₄: 560; found: 561 (M+H). ¹H NMR (400 MHz, DMSO-d₆) δ ppm: 8.15 (d, J=5.7 Hz, 1H), 7.42-7.32 (m, 1H), 7.22-7.06 (m, 3H), 6.91 (t, J=8.0 Hz, 1H), 6.79 (d, J=5.7 Hz, 1H), 6.71-6.63 (m, 1H), 6.59-6.52 (m, 1H), 6.42-6.34 (m, 1H), 5.66 (br s, 1H), 4.89-4.52 (br m, 1H), 4.10 (d, J=4.5 Hz, 2H), 3.49-3.37 (m, 2H), 2.99 (dd, J=15.7, 7.1 Hz, 1H), 2.80 (t, J=7.7 Hz, 2H), 2.70 (t, J=7.5 Hz, 2H), 2.60-2.51 (m, 1H), 2.43-2.32 (m, 1H), 2.23-2.08 (m, 2H), 2.01-1.44 (m, 9H), 1.44-1.32 (m, 1H), 1.29-1.14 (m, 1H).

The diastereoisomer pair eluting later was collected as Example 500 (contained 25% Example 499) and was isolated as a white solid (12 mg, 0.02 mmol, 3%). LRMS calculated for C₃₃H₃₇ClN₂O₄: 560; found: 561 (M+H). ¹H NMR (400 MHz, DMSO-d₆) δ ppm: 8.16 (d, J=5.7 Hz, 1H), 7.42-7.33 (m, 1H), 7.20-7.07 (m, 3H), 6.93 (t, J=8.0 Hz, 1H), 6.77 (d, J=5.7 Hz, 1H), 6.69 (t, J=2.2 Hz, 1H), 6.63-6.57 (m, 1H), 6.41-6.35 (m, 1H), 5.79 (br s, 1H), 4.77-4.56 (br m, 1H), 4.11 (dd, J=9.6, 5.5 Hz, 1H), 3.95 (dd, J=9.6, 6.4 Hz, 1H), 3.65-3.57 (m, 1H), 3.48-3.39 (m, 1H), 2.98 (dd, J=15.4, 6.8 Hz, 1H), 2.88-2.81 (m, 2H), 2.77-2.68 (m, 2H), 2.56-2.47 (m, 1H), 2.46-2.35 (m, 1H), 2.21-2.07 (m, 2H), 2.05-1.41 (m, 10H), 1.28-1.15 (m, 1H).

### Example 501

### Example 501A methyl (1r,4r)-2′-[2-({[tert-butyl(dimethyl)silyl]oxy}methyl)-3-hydroxypropyl]-4-(3-chloroanilino)-2′,3′-dihydrospiro[cyclohexane-1,1′-indene]-4-carboxylate

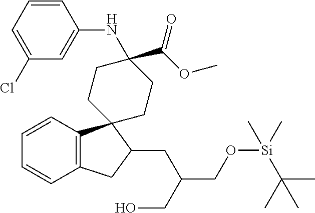

To a solution of Preparation 11b (570 mg, 1.24 mmol, 1 eq) in THE (9 mL), cooled to 0° C., was added NaH (60% dispersion; 30 mg, 1.24 mmol, 1 eq) and the mixture was stirred for 15 min. A solution of TBDMS-Cl (188 mg, 1.24 mmol, 1 eq) in THE (3 mL) was added and the mixture was allowed to warm to rt and stirred for 18 h. The mixture was partitioned between DCM and water. The phases were separated, and the organic phase was washed with brine, dried (MgSO₄) and concentrated in vacuo. Purification by automated flash chromatography (CombiFlash Rf, 24 g RediSep™ silica cartridge) eluting with a gradient of 0-40% EtOAc in heptane afforded a racemic mixture of diastereoisomers, Example 501A, isolated as a colourless gum (589 mg, 1.03 mmol, 83%). LRMS calculated for C₃₂H₄₆ClNO₄Si: 571; found: 572 (M+H).

### Example 501B methyl (1r,4r)-2′-{2-({[tert-butyl(dimethyl)silyl]oxy}methyl)-3-[(thieno[3,2-b]pyridin-7-yl)oxy]propyl}-4-(3-chloroanilino)-2′,3′-dihydrospiro[cyclohexane-1,1′-indene]-4-carboxylate

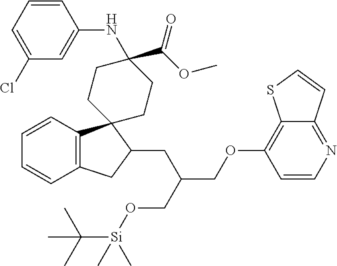

Using General Procedure 30b and Example 501A as the appropriate indane and thieno[3,2-b]pyridine-7-ol as the appropriate alcohol, a racemic mixture of diastereoisomers, Example 501B was isolated as a yellow gum (167 mg, 0.24 mmol, 90%). LRMS calculated for C₃₉H₄₉ClN₂O₄SiS: 704; found: 705 (M+H).

### Example 501C methyl (1r,4r)-4-(3-chloroanilino)-2′-{2-(hydroxymethyl)-3-[(thieno[3,2-b]pyridin-7-yl)oxy]propyl}-2′,3′-dihydrospiro[cyclohexane-1,1′-indene]-4-carboxylate

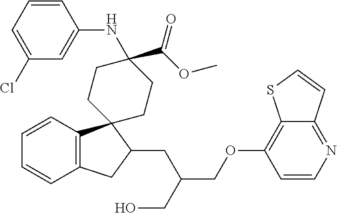

To a solution of Example 501B (167 mg, 0.24 mmol, 1 eq) in THE (5 mL) at 0° C. was added TBAF (1 M in THF, 0.28 mL, 0.28 mmol, 1.2 eq) and the mixture was stirred at rt for 1 h. The reaction was partitioned between DCM and water. The phases were separated, and the organic phase was washed with brine, dried (MgSO₄) and concentrated in vacuo. Purification by automated flash column chromatography (CombiFlash Rf, 12 g RediSep™ silica cartridge) eluting with a gradient of 0-8% MeOH in DCM afforded a racemic mixture of diastereoisomers, Example 501C, isolated as a yellow foam (124 mg, 0.21 mmol, 89%). LRMS calculated for C₃₃H₃₅ClN₂O₄S: 590; found: 591 (M+H).

### Example 501D methyl (1r,4r)-4-(3-chloroanilino)-2′-{2-(phenoxymethyl)-3-[(thieno[3,2-b]pyridin-7-yl)oxy]propyl}-2′,3′-dihydrospiro[cyclohexane-1,1′-indene]-4-carboxylate

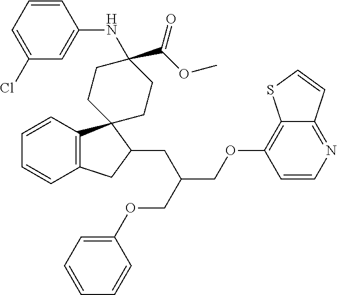

Using General Procedure 30a and Example 501C as the appropriate indane and phenol as the appropriate alcohol, a mixture of diastereoisomers, Example 501D, was isolated as a white solid (9.4 mg, 0.01 mmol, 7%). LRMS calculated for C₃₉H₃₉ClN₂O₄S: 666; found: 667 (M+H).

### Example 501 (1r,4r)-4-(3-chloroanilino)-2′-{2-(phenoxymethyl)-3-[(thieno[3,2-b]pyridin-7-yl)oxy]propyl}-2′,3′-dihydrospiro[cyclohexane-1,1′-indene]-4-carboxylic acid

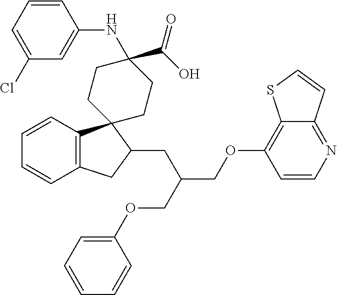

To a solution of Example 501D (9.4 mg, 0.01 mmol, 1 eq) in MeOH (2 mL) was added LiOH×H₂O (7 mg, 0.16 mmol, 6 eq) and the mixture was heated at 120° C. for 6 h under microwave irradiation. The mixture was concentrated in vacuo and suspended in a minimum amount of water, acidified with 2 M aq. HCl solution and the organics were extracted with DCM. The combined organic extracts were washed with brine, dried (MgSO₄) and concentrated in vacuo. Purification by automated flash chromatography (CombiFlash Rf, 4 g RediSep™ silica cartridge) eluting with a gradient of 0-14% MeOH in DCM afforded a solid. The solid was dissolved in MeOH, then loaded onto a DCM-wet PE-AX cartridge (5 g), washed successively with DCM, MeOH and eluted with 5% HCOOH in DCM, and concentrated in vacuo to afford Example 501 as a racemic mixture of diastereoisomers, isolated as a white powder (4.9 mg, 0.01 mmol, 53%). LRMS calculated for C₃₈H₃₇ClN₂O₄S: 652; found: 653 (M+H). ¹H NMR (400 MHz, DMSO-d₆) δ ppm: 8.52/8.51 (d, J=5.4 Hz, 1H), 8.03/7.89 (d, J=5.5 Hz, 1H), 7.51/7.48 (d, J=5.5 Hz, 1H), 7.43-7.35 (m, 1H), 7.31-7.11 (m, 5H), 7.09-7.01 (m, 2H), 7.01-6.87 (m, 3H), 6.65-6.49 (m, 3H), 4.51-4.33 (m, 2H), 4.22-4.08 (m, 2H), 3.13-3.03 (m, 1H), 2.69-2.35 (m, 2H), 2.31-1.33 (m, 11H).

### Example 502 and Example 503 and Example 504

### Example 502A methyl (1r,4r)-4-(3-chloroanilino)-2′-(3-oxopropyl)-2′,3′-dihydrospiro[cyclohexane-1,1′-indene]-4-carboxylate

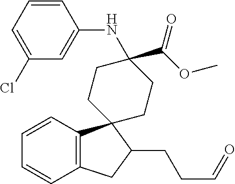

To a solution of Preparation 9b (410 mg, 0.96 mmol, 1 eq) in DCM (10 mL), cooled to 0° C., was added DMP (448 mg, 1.06 mmol, 1.1 eq) and the mixture was stirred at 0° C. for 1 h, then at rt for 18 h. The reaction was partitioned between DCM and sat. aq. NaHCO₃ solution, the aq. phase was extracted with DCM, and the combined organic extracts were washed with brine, dried (MgSO₄) and concentrated in vacuo. Purification by automated flash chromatography (CombiFlash Rf, 12 g RediSep™ silica cartridge) eluting with a gradient of 0-35% EtOAc in heptane afforded racemate Example 502A as a colourless oil (397 mg, 0.93 mmol, 97%). LRMS calculated for C₂₅H₂₈ClNO₃: 425; found: 426 (M+H). ¹H NMR (400 MHz, DMSO-d₆) δ ppm: 9.72 (t, J=1.4 Hz, 1H), 7.42-7.34 (m, 1H), 7.23-7.12 (m, 3H), 7.08 (t, J=8.1 Hz, 1H), 6.61 (t, J=2.1 Hz, 1H), 6.60-6.55 (m, 1H), 6.49-6.44 (m, 1H), 6.34 (s, 1H), 3.66 (s, 3H), 2.95 (dd, J=15.7, 7.2 Hz, 1H), 2.65-2.38 (m, 4H), 2.19-2.08 (m, 1H), 2.05-1.71 (m, 6H), 1.50-1.30 (m, 3H).

### Example 502 (1r,4r)-4-(3-chloroanilino)-2′-(2,2-difluoro-3-methoxypropyl)-2′,3′-dihydrospiro[cyclohexane-1,1′-indene]-4-carboxylic acid

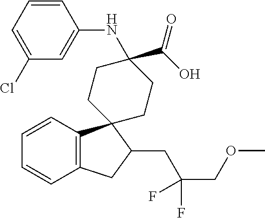

and

### Example 503 (1r,4r)-4-(3-chloroanilino)-2′-{3-[(6,7-dihydro-5H-cyclopenta[b]pyridin-4-yl)oxy]-2,2-difluoropropyl}-2′,3′-dihydrospiro[cyclohexane-1,1′-indene]-4-carboxylic acid

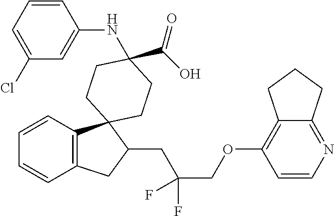

and

### Example 504 (1r,4r)-4-(3-chloroanilino)-2′-{3-[(6,7-dihydro-5H-cyclopenta[b]pyridin-4-yl)oxy]-2-fluoropropyl}-2′,3′-dihydrospiro[cyclohexane-1,1′-indene]-4-carboxylic acid

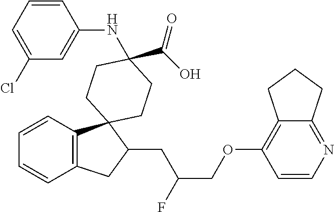

To a solution of DL-proline (43 mg, 0.37 mmol, 0.4 eq) in THE (3 mL) and iPrOH (0.3 mL) was added Example 502A (397 mg, 0.93 mmol, 1 eq). The mixture was stirred at rt for 18 h before the addition of N-fluorobenzenesulfonimide (588 mg, 1.86 mmol, 2 eq) and then stirred for a further 18 h. The reaction was partitioned between EtOAc and water, the organic phase was washed brine, dried (MgSO₄) and concentrated in vacuo. Purification by automated flash chromatography (CombiFlash Rf, 40 g RediSep™ silica cartridge) eluting with a gradient of 0-100% EtOAc in DCM afforded an intermediate, which was dissolved in MeOH (10 mL). NaBH₄ (27 mg, 0.72 mmol, 1 eq) was added and the mixture was stirred at rt for 18 h. The mixture was concentrated in vacuo and partitioned between DCM and water. The organic phase was washed with brine, dried (MgSO₄) and concentrated in vacuo. Purification by automated flash chromatography (CombiFlash Rf, 24 g RediSep™ silica cartridge) eluting with a gradient of 0-20% MeOH in DCM afforded a mixture of mono- and bis-fluorinated intermediate. It was treated as described in General procedure 31c, using 4-chloro-6,7-dihydro-5H-cyclopenta[b]pyridine (201 mg, 1.31 mmol, 2 eq) as the appropriate aryl chloride. The products were separated by preparative HPLC at pH 9 and pH 4 to obtain racemate Example 502 (28 mg, 0.06 mmol, 9%). LRMS calculated for C₂₅H₂₈ClF₂NO₃: 463; found: 464 (M+H). ¹H NMR (400 MHz, DMSO-d₆) δ ppm: 7.49-7.41 (m, 1H), 7.23-7.11 (m, 3H), 7.04 (t, J=8.1 Hz, 1H), 6.61 (t, J=2.1 Hz, 1H), 6.57-6.50 (m, 2H), 6.22 (br s, 1H), 3.17 (s, 3H), 2.97 (dd, J=15.7, 6.9 Hz, 1H), 2.62 (dd, J=15.7, 9.7 Hz, 1H), 2.49-2.38 (m, 1H), 2.24-1.72 (m, 9H), 1.67-1.56 (m, 1H), 1.47-1.38 (m, 1H), 1.28-1.16 (m, 1H).

Example 503 was isolated as a racemate (2.69 mg, 4.74 μmol, 1%). LRMS calculated for C₃₂H₃₃ClF₂N₂O₃: 566; found: 567 (M+H). ¹H NMR (400 MHz, DMSO-d₆) δ ppm: 8.20 (d, J=5.9 Hz, 1H), 7.53-7.45 (m, 1H), 7.24-7.08 (m, 3H), 7.01 (t, J=8.0 Hz, 1H), 6.89 (d, J=5.9 Hz, 1H), 6.67-6.63 (m, 1H), 6.59-6.53 (m, 1H), 6.51-6.45 (m, 1H), 6.09 (br s, 1H), 4.59-4.39 (m, 2H), 3.11-3.00 (m, 1H), 2.91-2.66 (m, 5H), 2.50-2.13 (m, 3H), 2.11-1.64 (m, 8H), 1.47-1.37 (m, 1H), 1.33-1.22 (m, 1H).

Example 504 was isolated as a racemic mixture of diastereoisomers (5.11 mg, 0.01 mmol, 1%). LRMS calculated for C₃₂H₃₄ClFN₂O₃: 548; found: 549 (M+H). ¹H NMR (400 MHz, DMSO-d₆) δ ppm: 8.18 (d, J=5.8 Hz, 1H), 7.44-7.37 (m, 1H), 7.25-7.11 (m, 3H), 7.00 (t, J=8.1 Hz, 1H), 6.82 (d, J=5.8 Hz, 1H), 6.69-6.65 (m, 1H), 6.60-6.55 (m, 1H), 6.50-6.45 (m, 1H), 6.14 (br s, 1H), 5.13-4.88 (m, 1H), 4.42-4.15 (m, 2H), 3.11-3.02 (m, 1H), 2.91-2.61 (m, 5H), 2.49-2.37 (m, 1H), 2.32-2.21 (m, 1H), 2.20-1.31 (m, 11H).

### Example 505 and Example 506

### Example 505A methyl (1r,4r)-4-(3-chloroanilino)-2′-(prop-2-en-1-yl)spiro[cyclohexane-1,1′-indene]-4-carboxylate

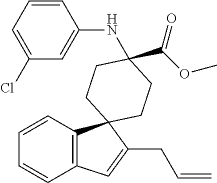 [SMILES: C=CCC1=Cc2ccccc2[C@]12CC[C@@](Nc1cccc(Cl)c1)(C(=O)OC)CC2]

Using General procedure 27a and Preparation 6a as the appropriate 2-bromo-indene derivative and allyl zinc bromide as the appropriate Zn reagent, Example 505A was obtained. ¹H NMR (500 MHz, DMSO-d₆) δ ppm: 7.70-7.10 (m, 4H), 7.09 (t, 1H), 6.64 (t, 1H), 6.59 (dm, 1H), 6.47 (dm, 1H), 6.42 (s, 1H), 6.40 (t, 1H), 5.97 (m, 1H), 5.18/5.12 (dm+dm, 2H), 3.70 (s, 3H), 2.99 (m, 2H), 2.40/2.20 (m+m, 4H), 2.14/0.91 (m+m, 4H).

### Example 505B methyl (1r,4r)-4-(3-chloroanilino)-2′-(2,3-dihydroxypropyl)spiro[cyclohexane-1,1′-indene]-4-carboxylate

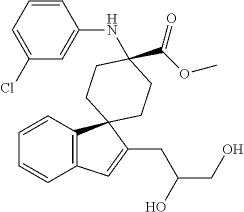 [SMILES: COC(=O)[C@]1(Nc2cccc(Cl)c2)CC[C@]2(CC1)C(CC(O)CO)=Cc1ccccc12]

Example 505A (3.58 g, 8.78 mmol) was dissolved in acetone (79 mL) and water (9 mL), then 4-methyl-4-oxido-morpholin-4-ium (1.54 g, 13.2 mmol) and then OsO₄ (854 mg, 0.105 mmol) was added at rt, and the mixture was stirred at rt for 8 h. Then it was quenched with sat. aq. Na₂SO₃ solution, and the volatiles were evaporated under reduced pressure. Then it was extracted with EtOAc, dried over MgSO₄, filtered and the filtrate was concentrated under reduced pressure. The crude product was purified via flash chromatography using heptane and EtOAc as eluents to obtain Example 505B (1.36 g, 3.08 mmol, 35%). ¹H NMR (500 MHz, DMSO-d₆) δ ppm: 7.64 (d, 1H), 7.28 (d, 1H), 7.22 (t, 1H), 7.10 (td, 1H), 7.09 (t, 1H), 6.63 (t, 1H), 6.58 (dd, 1H), 6.57 (br s, 1H), 6.47 (dd, 1H), 6.44 (s, 1H), 4.66 (d, 1H), 4.59 (t, 1H), 3.82 (m, 1H), 3.69 (s, 3H), 3.36 (m, 2H), 2.41/2.17 (m+m, 2H), 2.40/2.21/2.11/0.87 (m+m+m+m, 8H).

### Example 505C methyl (1r,4r)-4-(3-chloroanilino)-2′-(2-hydroxy-3-{[tri(propan-2-yl)silyl]oxy}propyl)spiro[cyclohexane-1,1′-indene]-4-carboxylate

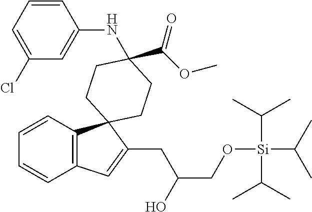 [SMILES: COC(=O)[C@]1(Nc2cccc(Cl)c2)CC[C@]2(CC1)C(CC(O)CO[Si](C(C)C)(C(C)C)C(C)C)=Cc1ccccc12]

To a solution of Example 505B (507 mg, 1.15 mmol, 1 eq) and 2,6-lutidine (174 μL, 1.49 mmol, 1.3 eq) in DCM (15 mL) cooled to 0° C. under N₂, was added triisopropylsilyl trifluoromethanesulfonate (339 μL, 1.26 mmol, 1.1 eq) and the mixture was stirred at rt for 12 h. The mixture was partitioned between DCM and water and the organic phase dried (PTFE phase separator) and concentrated in vacuo. Purification by automated flash chromatography (Combiflash Rf, Silica 12 g RediSep™ cartridge) eluting with a gradient of 0-30% EtOAc in heptane afforded racemate Example 505C as a colourless gum (542 mg, 0.91 mmol, 79%). LRMS calculated for C₃₄H₄₈NO₄SiCl: 597; found: 598 (M+H). ¹H NMR (400 MHz, DMSO-d₆) δ ppm: 7.65 (d, J=7.5 Hz, 1H), 7.29 (dd, J=7.5, 1.3 Hz, 1H), 7.22 (td, J=7.4, 1.0 Hz, 1H), 7.14-7.04 (m, 2H), 6.64 (t, J=2.1 Hz, 1H), 6.63-6.56 (m, 2H), 6.46-6.40 (m, 2H), 4.75 (d, J=5.3 Hz, 1H), 3.90-3.82 (m, 1H), 3.71-3.66 (m, 4H), 3.53 (dd, J=9.7, 6.5 Hz, 1H), 2.48-2.32 (m, 2H), 2.29-2.04 (m, 5H), 1.06-0.96 (m, 21H), 0.93-0.81 (m, 2H).

### Example 505D methyl (1r,4r)-4-(3-chloroanilino)-2′-(2-fluoro-3-{[tri(propan-2-yl)silyl]oxy}propyl)spiro[cyclohexane-1,1′-indene]-4-carboxylate

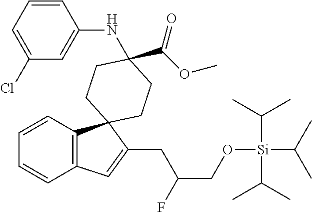 [SMILES: COC(=O)[C@]1(Nc2cccc(Cl)c2)CC[C@]2(CC1)C(CC(F)CO[Si](C(C)C)(C(C)C)C(C)C)=Cc1ccccc12]

To a solution of Example 505C (540 mg, 0.9 mmol, 1 eq) in DCM (15 mL) cooled to 0° C. under N₂ was added DAST (179 μL, 1.35 mmol, 1.5 eq) dropwise and the mixture was stirred for 1 h. The mixture was cooled to 0° C. and carefully quenched by the dropwise addition of 2 M aq. NaOH solution. The mixture was diluted with DCM and the organic phase dried (PTFE phase separator) and concentrated in vacuo to afford racemate Example 505D as a cream solid (570 mg, 0.95 mmol). LRMS calculated for C₃₄H₄₇NO₃FSiCl: 599; found: 600 (M+H). ¹H NMR (400 MHz, DMSO-d₆) δ ppm: 7.68 (d, J=7.5 Hz, 1H), 7.32 (dd, J=7.4, 1.3 Hz, 1H), 7.25 (td, J=7.4, 0.9 Hz, 1H), 7.14 (td, J=7.5, 1.4 Hz, 1H), 7.08 (t, J=8.1 Hz, 1H), 6.65 (t, J=2.1 Hz, 1H), 6.63-6.57 (m, 2H), 6.47-6.41 (m, 2H), 5.04-4.85 (m, 1H), 3.99-3.80 (m, 2H), 3.70 (s, 3H), 2.70-2.56 (m, 2H), 2.47-2.32 (m, 2H), 2.29-2.06 (m, 4H), 1.06-0.84 (m, 23H).

### Example 505E methyl (1r,4r)-4-(3-chloroanilino)-2′-(2-fluoro-3-hydroxypropyl)spiro[cyclohexane-1,1′-indene]-4-carboxylate

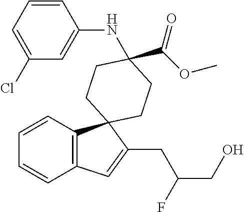 [SMILES: COC(=O)[C@]1(Nc2cccc(Cl)c2)CC[C@]2(CC1)C(CC(F)CO)=Cc1ccccc12]

Using General procedure 29 and Example 505D (165 mg, 0.27 mmol, 1 eq) as the appropriate silyl derivative, racemate Example 505E was obtained as a cream solid (106 mg, 0.24 mmol, 87%). LRMS calculated for C₂₅H₂₇NO₃FCl: 443; found: 444 (M+H). ¹H NMR (400 MHz, DMSO-d₆) δ ppm: 7.67 (d, J=7.5 Hz, 1H), 7.33 (dd, J=7.5, 1.3 Hz, 1H), 7.25 (td, J=7.5, 0.9 Hz, 1H), 7.14 (td, J=7.5, 1.4 Hz, 1H), 7.10 (t, 1H), 6.64 (t, J=2.1 Hz, 1H), 6.62-6.58 (m, 2H), 6.51-6.46 (m, 1H), 6.45 (s, 1H), 5.05 (t, J=5.7 Hz, 1H), 4.97-4.78 (m, 1H), 3.70 (s, 3H), 3.69-3.52 (m, 2H), 2.60-2.55 (m, 1H), 2.47-2.35 (m, 2H), 2.27-2.05 (m, 4H), 0.96-0.85 (m, 2H).

### Example 505F 5,6,7,8-tetrahydroquinolin-4-ol

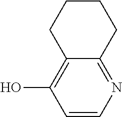 [SMILES: Oc1ccnc2c1CCCC2]

To an oven-dried microwave vial was added 4-chloro-5,6,7,8-tetrahydroquinoline (500 mg, 2.98 mmol, 1 eq), K₂CO₃ (1.24 g, 8.95 mmol, 3 eq), tBuXPhos (101 mg, 0.24 mmol, 0.08 eq) and Herrmann's catalyst (56 mg, 0.06 mmol, 0.02 eq) in DMF (5 mL) and water (0.5 mL). The mixture was sparged with N₂ (10 min) and heated at 115° C. for 2 h under microwave irradiation. The reaction was allowed to cool to rt, partitioned between DCM/iPrOH (3:1) and sat. aq. NaHCO₃ solution, and the organic phase was washed with brine, dried (MgSO₄) and concentrated in vacuo. Purification by automated flash chromatography (CombiFlash Rf, 12 g RediSep™ silica cartridge) eluting with a gradient of 0-20% MeOH in DCM afforded Example 505F as a cream solid (237 mg, 1.59 mmol, 53%). LRMS calculated for C₉H₁₁NO: 149; found: 150 (M+H). ¹H NMR (400 MHz, DMSO-d₆) δ ppm: 11.07 (s, 1H), 7.46 (d, J=7.2 Hz, 1H), 5.91 (d, J=7.2 Hz, 1H), 2.32-2.21 (m, 2H), 1.75-1.58 (m, 4H).

### Example 505 (1r,4r)-4-(3-chloroanilino)-2′-{2-fluoro-3-[(5,6,7,8-tetrahydroquinolin-4-yl)oxy]propyl}spiro[cyclohexane-1,1′-indene]-4-carboxylic acid, enantiomer 1

and

### Example 506 (1r,4r)-4-(3-chloroanilino)-2′-{2-fluoro-3-[(5,6,7,8-tetrahydroquinolin-4-yl)oxy]propyl}spiro[cyclohexane-1,1′-indene]-4-carboxylic acid, enantiomer 2

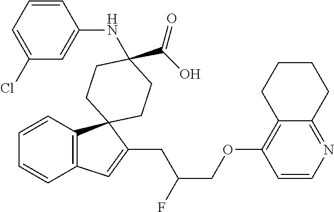 [SMILES: O=C(O)[C@]1(Nc2cccc(Cl)c2)CC[C@]2(CC1)C(CC(F)COc1ccnc3c1CCCC3)=Cc1ccccc12]

Using General procedure 30a and Example 505E (105 mg, 0.24 mmol, 1 eq) as the appropriate indene and Example 505F (71 mg, 0.47 mmol, 2 eq) as the appropriate alcohol afforded an intermediate which was hydrolyzed according to General procedure 33a, yielding a racemate (63.8 mg, 0.11 mmol, 48%). The enantiomers were separated by chiral chromatography. Column: AD, 50×500 mm, 20 μm, Eluents: iPrOH+0.1% HCOOH. The enantiomer eluting earlier was purified via prep RP-HPLC using 25 mM aq. NH₄HCO₃ solution and MeCN as eluents resulting in Example 505. ¹H NMR (500 MHz, DMSO-d₆) δ ppm: 8.16 (d, 1H), 7.68/7.32/7.24/7.15 (d+d+t+t, 4H), 7.08 (t, 1H), 6.81 (d, 1H), 6.68 (s, 1H), 6.64 (t, 1H), 6.56 (m, 1H), 6.56 (m, 1H), 6.36 (br s, 1H), 5.32 (dm, 1H), 4.41-4.21 (m, 2H), 2.83-2.65 (m, 2H), 2.73 (m, 2H), 2.55 (m, 2H), 2.4/2.19 (m+m, 4H), 2.12/0.9 (m+m, 4H), 1.72 (m, 2H), 1.64 (m, 2H). HRMS calculated for C₃₃H₃₄N₂O₃FCl: 560.2242; found: 561.2320 (M+H).

The enantiomer eluting later was purified via prep RP-HPLC using 25 mM aq. NH₄HCO₃ solution and MeCN as eluents resulting in Example 506. HRMS calculated for C₃₃H₃₄N₂O₃FCl: 560.2242; found: 561.2308 (M+H).

### Example 507 and Example 508 and Example 509 and Example 510

### Example 507A methyl (1r,4r)-4-(3-chloroanilino)-2′-(2-fluoro-3-{[tri(propan-2-yl)silyl]oxy}propyl)-2′,3′-dihydrospiro[cyclohexane-1,1′-indene]-4-carboxylate

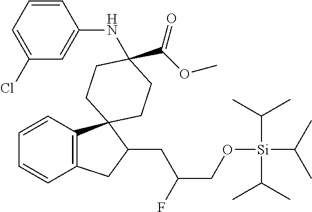

To a solution of Example 505D (400 mg, 0.67 mmol, 1 eq) in EtOAc (10 mL) was added 5% Pt/C (7 mg, 0.03 mmol, 0.05 eq) in iPrOH (1 mL) under a N₂ atmosphere. The mixture was evacuated and backfilled with N₂ (×3), then evacuated and backfilled with H₂ and shaken for 2 days at rt under an atmosphere of H₂. The reaction was filtered through celite, further 5% Pt/C (7 mg, 0.03 mmol, 0.05 eq) was added and the mixture was heated at 45° C. for 48 h under an atmosphere of H₂. This was repeated until complete conversion was observed. The reaction was filtered through celite and the solvents removed in vacuo to afford a racemic mixture of diastereoisomers, Example 507A as a colourless gum (323 mg, 0.54 mmol, 80%) that was used directly in the subsequent step without further purification. LRMS calculated for C₃₄H₄₉NO₃FSiCl: 601; found: 602 (M+H).

### Example 507B methyl (1r,4r)-4-(3-chloroanilino)-2′-(2-fluoro-3-hydroxypropyl)-2′,3′-dihydrospiro[cyclohexane-1,1′-indene]-4-carboxylate

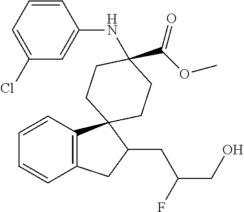

Using General procedure 29 and Example 507A as the appropriate silyl derivative (323 mg, 0.54 mmol, 1 eq), a racemic mixture of diastereoisomers, Example 507B was obtained as a white powder (180 mg, 0.4 mmol, 75%). LRMS calculated for C₂₅H₂₉NO₃FCl: 445; found: 446 (M+H). ¹H NMR (400 MHz, DMSO-d₆) δ ppm: 7.46-7.35 (m, 1H), 7.26-7.12 (m, 3H), 7.11-7.04 (m, 1H), 6.62-6.53 (m, 2H), 6.49-6.42 (m, 1H), 6.35/6.32 (s, 1H), 4.94 (t, J=5.6 Hz, 1H), 4.71-4.49 (m, 1H), 3.69-3.46 (m, 5H), 3.08-2.94 (m, 1H), 2.70-2.56 (m, 1H), 2.49-2.37 (m, 1H), 2.27-1.64 (m, 7H), 1.60-1.21 (m, 3H).

### Example 507C methyl (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylate, diastereoisomer 1

### Example 508A methyl (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylate, diastereoisomer 2

### Example 509A methyl (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylate, diastereoisomer 3

### Example 510A methyl (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylate, diastereoisomer 4

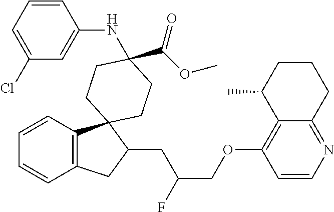

Using General procedure 30a and Example 507B (180 mg, 0.4 mmol, 1 eq) as the appropriate indane and Preparation 2a1 (138 mg, 0.85 mmol, 2.1 eq) as the appropriate alcohol, a racemic mixture of diastereoisomers was obtained (139 mg, 0.24 mmol, 58%). The diastereoisomers were separated by chiral chromatography. Column: AD, 50×500 mm, 20 μm, Eluents: 50:50 EtOH/heptane. The diastereoisomer eluting firstly was collected as Example 507C. ¹H NMR (500 MHz, DMSO-d₆) δ ppm: 8.18 (d, 1H), 7.43-7.12 (m, 4H), 7.06 (t, 1H), 6.82 (d, 1H), 6.61 (t, 1H), 6.57 (dm, 1H), 6.44 (dm, 1H), 6.35 (s, 1H), 5.02 (dm, 1H), 4.29/4.24 (dd+dd, 2H), 3.65 (s, 3H), 3.1 (m, 1H), 3.09/2.68 (dd+dd, 2H), 2.78/2.68 (dm+m, 2H), 2.5-1.34 (m, 10H), 2.29 (m, 1H), 1.86/1.7 (m+m, 2H), 1.65/1.26 (m+m, 2H), 1.14 (d, 3H). HRMS calculated for C₃₅H₄₀ClFN₂O₃: 590.2712; found: 591.2795 (M+H).

The diastereoisomer eluting secondly was collected as Example 508A. ¹H NMR (500 MHz, DMSO-d₆) δ ppm: 8.18 (d, 1H), 7.48-7.12 (m, 4H), 7.05 (t, 1H), 6.84 (d, 1H), 6.59 (t, 1H), 6.57 (dm, 1H), 6.41 (dm, 1H), 6.28 (s, 1H), 5.04 (dm, 1H), 4.29/4.24 (dd+dd, 2H), 3.65 (s, 3H), 3.07 (m, 1H), 3.03/2.7 (dd+dd, 2H), 2.77/2.68 (dm+m, 2H), 2.5-1.26 (m, 10H), 2.14 (m, 1H), 1.78/1.73 (m+m, 2H), 1.65/1.58 (m+m, 2H), 1.07 (d, 3H). HRMS calculated for C₃₅H₄₀ClFN₂O₃: 590.2712; found: 591.2780 (M+H).

The diastereoisomer eluting thirdly was collected as Example 509A. ¹H NMR (500 MHz, DMSO-d₆) δ ppm: 8.16 (d, 1H), 7.48-7.12 (m, 4H), 7.06 (t, 1H), 6.82 (d, 1H), 6.6 (t, 1H), 6.57 (dm, 1H), 6.43 (dm, 1H), 6.3 (s, 1H), 5.03 (dm, 1H), 4.33/4.23 (dd+dd, 2H), 3.64 (s, 3H), 3.03/2.69 (dd+dd, 2H), 3.03 (m, 1H), 2.75/2.64 (dm+m, 2H), 2.5-1.28 (m, 10H), 2.08 (m, 1H), 1.78/1.68 (m+m, 2H), 1.58/1.48 (m+m, 2H), 1.11 (d, 3H). HRMS calculated for C₃₅H₄₀ClFN₂O₃: 590.2712; found: 591.2777 (M+H).

The diastereoisomer eluting fourthly was collected as Example 510A. ¹H NMR (500 MHz, DMSO-d₆) δ ppm: 8.2 (d, 1H), 7.43-7.12 (m, 4H), 7.06 (t, 1H), 6.86 (d, 1H), 6.6 (t, 1H), 6.57 (dm, 1H), 6.46 (dm, 1H), 6.37 (s, 1H), 5.01 (dm, 1H), 4.36/4.19 (dd+dd, 2H), 3.65 (s, 3H), 3.09 (m, 1H), 3.08/2.67 (dd+dd, 2H), 2.79/2.69 (dm+m, 2H), 2.5-1.38 (m, 10H), 2.28 (m, 1H), 1.81/1.75 (m+m, 2H), 1.7/1.62 (m+m, 2H), 1.14 (d, 3H). HRMS calculated for C₃₅H₄₀ClFN₂O₃: 590.2712; found: 591.2772 (M+H).

### Example 507 (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylic acid, diastereoisomer 1

and

### Example 508 (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylic acid, diastereoisomer 2

and

### Example 509 (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylic acid, diastereoisomer 3

and

### Example 510 (1r,4R)-4-(3-chloroanilino)-2′-(2-fluoro-3-{[(5R)-5-methyl-5,6,7,8-tetrahydroquinolin-4-yl]oxy}propyl)-2′,3′-dihydrospiro[cyclohexane-1,1′-indene]-4-carboxylic acid, diastereoisomer 4

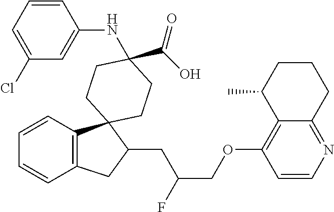

Using General procedure 33a and Example 507C as the appropriate ester Example 507 was obtained. ¹H NMR (500 MHz, DMSO-d₆) δ ppm: 12.76 (br s, 1H), 8.17 (d, 1H), 7.38 (d, 1H), 7.22 (d, 1H), 7.18 (t, 1H), 7.16 (t, 1H), 7.05 (t, 1H), 6.81 (d, 1H), 6.64 (dd, 1H), 6.55 (dd, 1H), 6.53 (dd, 1H), 6.25 (br s, 1H), 5.02 (m, 1H), 4.28/4.21 (dd+dd, 2H), 3.1 (m, 1H), 3.09/2.68 (dd+dd, 2H), 2.78/2.66 (dd+dd, 2H), 2.48-1.42 (m, 10H), 2.3 (m, 1H), 1.83/1.73 (m+m, 2H), 1.7/1.63 (m+m, 2H), 1.14 (d, 3H). HRMS calculated for C₃₄H₃₈N₂O₃FCl: 576.2555; found: 577.2623 (M+H).

Using General procedure 33a and Example 508A as the appropriate ester Example 508 was obtained. ¹H NMR (500 MHz, DMSO-d₆) δ ppm: 12.73 (br s, 1H), 8.17 (d, 1H), 7.5-7.12 (m, 4H), 7.04 (t, 1H), 6.83 (d, 1H), 6.61 (t, 1H), 6.57 (dm, 1H), 6.50 (dm, 1H), 6.19 (br s, 1H), 5.04 (dm, 1H), 4.35-4.17 (m, 2H), 3.07 (m, 1H), 3.03/2.7 (dd+dd, 2H), 2.77/2.67 (dm+m, 2H), 2.5-1.26 (m, 10H), 2.14 (m, 1H), 1.86-1.66 (m, 2H), 1.66/1.58 (m+m, 2H), 1.08 (d, 3H). HRMS calculated for C₃₄H₃₈N₂O₃FCl: 576.2555; found: 577.2614 (M+H).

Using General procedure 33a and Example 509A as the appropriate ester Example 509 was obtained. ¹H NMR (500 MHz, DMSO-d₆) δ ppm: 12.72 (br s, 1H), 8.16 (d, 1H), 7.46-7.12 (m, 4H), 7.05 (t, 1H), 6.82 (d, 1H), 6.62 (t, 1H), 6.55 (dm, 1H), 6.52 (dm, 1H), 6.19 (br s, 1H), 5.03 (dm, 1H), 4.4-4.15 (m, 2H), 3.05 (m, 1H), 3.02/2.69 (dd+dd, 2H), 2.75/2.63 (dm+m, 2H), 2.5-1.27 (m, 10H), 2.07 (m, 1H), 1.78/1.68 (m+m, 2H), 1.58/1.49 (m+m, 2H), 1.12 (d, 3H). HRMS calculated for C₃₄H₃₈N₂O₃FCl: 576.2555; found: 577.2626 (M+H).

Using General procedure 33a and Example 510A as the appropriate ester Example 510 was obtained. ¹H NMR (500 MHz, DMSO-d₆) δ ppm: 12.73 (br s, 1H), 8.17 (d, 1H), 7.41-7.12 (m, 4H), 7.05 (t, 1H), 6.81 (d, 1H), 6.63 (t, 1H), 6.55 (dm, 1H), 6.55 (dm, 1H), 6.27 (br s, 1H), 5.01 (dm, 1H), 4.4-4.1 (m, 2H), 3.09 (m, 1H), 3.08/2.67 (dd+dd, 2H), 2.78/2.68 (dm+m, 2H), 2.49-1.4 (m, 10H), 2.29 (m, 1H), 1.82/1.74 (m+m, 2H), 1.69/1.61 (m+m, 2H), 1.14 (d, 3H). HRMS calculated for C₃₄H₃₈N₂O₃FCl: 576.2555; found: 577.2622 (M+H).

### Example 511

### Example 511A methyl (1r,4r)-2′-[2-({[tert-butyl(dimethyl)silyl]oxy}methyl)-3-oxopropyl]-4-(3-chloroanilino)-2′,3′-dihydrospiro[cyclohexane-1,1′-indene]-4-carboxylate

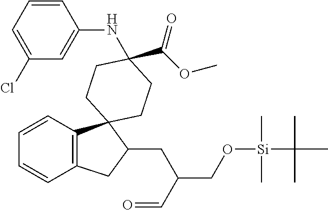

To a solution of Example 501A (426 mg, 0.74 mmol, 1 eq) in DCM (15 mL), cooled to 0° C., was added DMP (316 mg, 0.74 mmol, 1 eq) and the mixture was stirred at rt for 18 h. The mixture was quenched with 10% aq. Na₂S₂O₃ solution, partitioned between DCM and sat. aq. NaHCO₃ solution, and the organic phase was washed with brine, dried (MgSO₄) and concentrated in vacuo. Purification by automated flash chromatography (CombiFlash Rf, 12 g RediSep™ silica cartridge) eluting with a gradient of 0-25% EtOAc in heptane afforded a racemic mixture of diastereoisomers, Example 511A as a colourless oil (406 mg, 0.71 mmol, 96%). LRMS calculated for C₃₂H₄₄ClNO₄Si: 569; found: 570 (M+H). ¹H NMR (400 MHz, DMSO-d₆) δ ppm: 9.69-9.64 (m, 1H), 7.44-7.36 (m, 1H), 7.23-7.12 (m, 3H), 7.06 (t, J=8.1 Hz, 1H), 6.63-6.55 (m, 2H), 6.47-6.41 (m, 1H), 6.35/6.30 (s, 1H), 4.04-3.82 (m, 2H), 3.65 (s, 3H), 2.99-2.88 (m, 1H), 2.63-2.37 (m, 2H), 2.17-1.18 (m, 11H), 0.83/0.78 (s, 9H), 0.03/0.02/0.02/0.00 (s, 6H).

### Example 511B methyl (1r,4r)-2′-[2-({[tert-butyl(dimethyl)silyl]oxy}methyl)-3,3-difluoropropyl]-4-(3-chloroanilino)-2′,3′-dihydrospiro[cyclohexane-1,1′-indene]-4-carboxylate

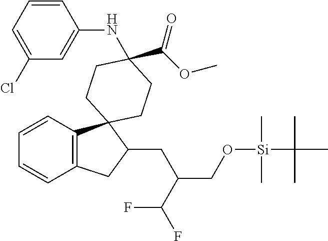

To a solution of Example 511A (491 mg, 0.86 mmol, 1 eq) in DCM (10 mL), cooled to 0° C., was added DAST (228 μL, 1.72 mmol, 2 eq) dropwise. The mixture was slowly warmed to rt and stirred for 18 h. The mixture was cooled to 0° C., quenched by the dropwise addition of 2 M aq. NaOH solution and then partitioned between DCM and water. The organic phase was washed with brine, dried (MgSO₄) and concentrated in vacuo. Purification by automated flash chromatography (CombiFlash Rf, 12 g RediSep™ silica cartridge) eluting with a gradient of 0-30% EtOAc in heptane afforded a racemic mixture of diastereoisomers, Example 511B as a yellow oil (450 mg, 0.76 mmol, 88%). LRMS C₃₂H₄₄ClF₂NO₃Si: 591; found: 592 (M+H). ¹H NMR (400 MHz, DMSO-d₆) δ ppm: 7.43-7.36 (m, 1H), 7.24-7.12 (m, 3H), 7.09-7.03 (m, 1H), 6.63-6.55 (m, 2H), 6.47-6.41 (m, 1H), 6.35/6.29 (s, 1H), 6.24-5.83 (m, 1H), 3.78-3.58 (m, 5H), 3.00 (dd, J=15.6, 7.0 Hz, 1H), 2.61-2.36 (m, 2H), 2.21-1.56 (m, 8H), 1.49-1.27 (m, 3H), 0.83/0.80 (s, 9H), 0.03/0.02/0.01/0.00 (s, 6H).

### Example 511C methyl (1r,4r)-4-(3-chloroanilino)-2′-[3,3-difluoro-2-(hydroxymethyl)propyl]-2′,3′-dihydrospiro[cyclohexane-1,1′-indene]-4-carboxylate

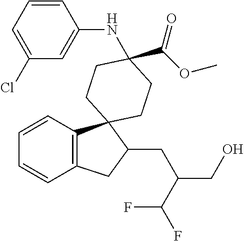

Using General procedure 29 and Example 511B (450 mg, 0.76 mmol, 1 eq) as the appropriate silyl derivative, and then purification by automated flash chromatography (CombiFlash Rf, 12 g RediSep™ silica cartridge) eluting with a gradient of 0-10% MeOH in DCM, a racemic mixture of diastereoisomers, Example 511C was obtained as a white foam (251 mg, 0.53 mmol, 69%). LRMS calculated for C₂₆H₃₀ClF₂NO₃: 477; found: 478 (M+H). ¹H NMR (400 MHz, DMSO-d₆) δ ppm: 7.41-7.35 (m, 1H), 7.24-7.13 (m, 3H), 7.07 (t, J=8.0 Hz, 1H), 6.62-6.55 (m, 2H), 6.49-6.44 (m, 1H), 6.36/6.33 (s, 1H), 6.27-5.90 (m, 1H), 4.86-4.82 (m, 1H), 3.68-3.40 (m, 5H), 3.06-2.96 (m, 1H), 2.62-2.37 (m, 2H), 2.23-1.82 (m, 6H), 1.79-1.33 (m, 4H), 1.33-1.18 (m, 1H).

### Example 511D methyl (1r,4r)-4-(3-chloroanilino)-2′-(3,3-difluoro-2-{[(thieno[3,2-b]pyridin-7-yl)oxy]methyl}propyl)-2′,3′-dihydrospiro[cyclohexane-1,1′-indene]-4-carboxylate

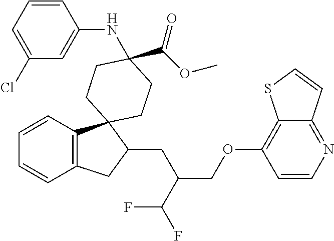

Using General procedure 30a and Example 511C (125 mg, 0.26 mmol, 1 eq) as the appropriate indane and thieno[3,2-b]pyridine-7-ol (119 mg, 0.78 mmol, 3 eq) as the appropriate alcohol, a racemic mixture of diastereoisomers, Example 511D was obtained as a cream foam (123 mg, 0.2 mmol, 77%). LRMS calculated for C₃₃H₃₃ClF₂N₂O₃S: 610; found: 611 (M+H). ¹H NMR (400 MHz, DMSO-d₆) δ ppm: 8.54/8.53 (d, J=5.4 Hz, 1H), 8.04/7.87 (d, J=5.5 Hz, 1H), 7.52/7.48 (d, J=5.5 Hz, 1H), 7.43-7.37 (m, 1H), 7.27-7.13 (m, 3H), 7.10-7.02 (m, 2H), 6.63-6.55 (m, 2H), 6.50-6.10 (m, 3H), 4.48-4.31 (m, 2H), 3.65/3.64 (s, 3H), 3.15-3.04 (m, 1H), 2.69-2.36 (m, 3H), 2.31-1.72 (m, 7H), 1.60-1.33 (m, 3H).

### Example 511 (1r,4r)-4-(3-chloroanilino)-2′-(3,3-difluoro-2-{[(thieno[3,2-b]pyridin-7-yl)oxy]methyl}propyl)-2′,3′-dihydrospiro[cyclohexane-1,1′-indene]-4-carboxylic acid

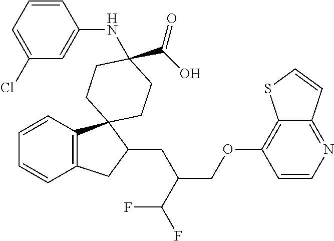

Using General procedure 33b and Example 511D (123 mg, 0.2 mmol, 1 eq) as the appropriate ester, a racemic mixture of diastereoisomers, Example 511 was obtained as a white powder (59.7 mg, 0.1 mmol, 50%). LRMS calculated for C₃₂H₃₁ClF₂N₂O₃S: 596; found: 597 (M+H). ¹H NMR (400 MHz, DMSO-d₆) δ ppm: 12.72 (br s, 1H), 8.54/8.53 (d, J=5.4 Hz, 1H), 8.04/7.88 (d, J=5.5 Hz, 1H), 7.52/7.49 (d, J=5.5 Hz, 1H), 7.43-7.36 (m, 1H), 7.26-7.12 (m, 3H), 7.10-7.02 (m, 2H), 6.66-6.48 (m, 3H), 6.48-6.09 (m, 1H), 4.48-4.31 (m, 2H), 3.14-3.04 (m, 1H), 2.69-2.35 (m, 3H), 2.31-1.72 (m, 7H), 1.60-1.35 (m, 3H).

### Example 512 (1r,4r)-4-(3-chloroanilino)-2′-(3,3-difluoro-2-{[(5,6,7,8-tetrahydroquinolin-4-yl)oxy]methyl}propyl)-2′,3′-dihydrospiro[cyclohexane-1,1′-indene]-4-carboxylic acid, diastereoisomer 1

and

### Example 513 (1r,4r)-4-(3-chloroanilino)-2′-(3,3-difluoro-2-{[(5,6,7,8-tetrahydroquinolin-4-yl)oxy]methyl}propyl)-2′,3′-dihydrospiro[cyclohexane-1,1′-indene]-4-carboxylic acid, diastereoisomer 2

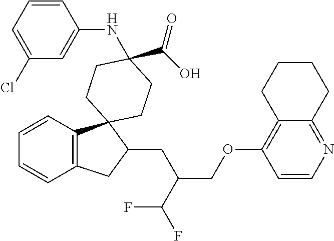

Using General procedure 30a and Example 511C (118 mg, 0.25 mmol, 1 eq) as the appropriate indane and Example 505F (73.7 mg, 0.49 mmol, 2 eq) as the appropriate alcohol, purification by automated flash chromatography (CombiFlash Rf, 12 g RediSep™ silica cartridge) eluting with a gradient of 0-15% MeOH in DCM, afforded an intermediate which was hydrolyzed according to General procedure 33b. The diastereoisomers were partially separated. The firstly eluted peak yielded a 3:2 mixture of diastereoisomers, collected as Example 512 as a white powder (7.7 mg, 0.01 mmol, 5%). LRMS calculated for C₃₄H₃₇ClF₂N₂O₃: 594; found: 595 (M+H). ¹H NMR (400 MHz, DMSO-d₆) δ ppm: 12.71 (br s, 1H), 8.24-8.20 (m, 1H), 7.42-7.34 (m, 1H), 7.26-7.13 (m, 3H), 7.05 (t, J=8.1 Hz, 1H), 6.93-6.85 (m, 1H), 6.65-6.49 (m, 3H), 6.45-6.06 (m, 1H), 4.26-4.06 (m, 2H), 3.10-3.01 (m, 1H), 2.78-2.35 (m, 7H), 2.29-2.06 (m, 2H), 2.05-1.52 (m, 9H), 1.52-1.33 (m, 3H).

The secondly eluting peak was loaded onto a DCM-wet PE-AX cartridge (20 g), washed successively with DCM, MeOH and eluted with 5% HCOOH in DCM, and concentrated in vacuo. Purification by automated flash chromatography (CombiFlash Rf, 12 g RediSep™ silica cartridge) eluting with a gradient of 0-20% MeOH in DCM afforded a 1:3 mixture of diastereoisomers, collected as Example 513 as a white powder (3.5 mg, 0.01 mmol, 2%). LRMS calculated for C₃₄H₃₇ClF₂N₂O₃: 594: found: 595 (M+H). ¹H NMR (400 MHz, DMSO-d₆) δ ppm: 8.19-8.15 (m, 1H), 7.43-7.34 (m, 1H), 7.25-7.11 (m, 3H), 7.08-6.98 (m, 1H), 6.84-6.78 (m, 1H), 6.70-6.48 (m, 3H), 6.44-6.06 (m, 1H), 4.20-4.02 (m, 2H), 3.09-3.01 (m, 1H), 2.76-2.66 (m, 2H), 2.65-2.36 (m, 5H), 2.32-2.07 (m, 2H), 2.03-1.32 (m, 12H).

### Example 514 and Example 515 Example 514A methyl (1r,4r)-2′-[2-({[tert-butyl(dimethyl)silyl]oxy}methyl)-3-hydroxypropyl]-4-(3-chloroanilino)spiro[cyclohexane-1,1′-indene]-4-carboxylate

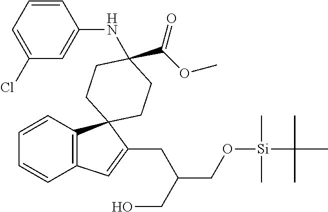 [SMILES: COC(=O)[C@]1(Nc2cccc(Cl)c2)CC[C@]2(CC1)C(CC(CO)CO[Si](C)(C)C(C)(C)C)=Cc1ccccc12]

Using General procedure 41a and Preparation 11a (267 mg, 0.59 mmol, 1 eq) as the appropriate alcohol and TBDMS-Cl (88.26 mg, 0.59 mmol, 1 eq) as the appropriate silyl chloride, racemate Example 514A was obtained as a colourless gum (230 mg, 0.4 mmol, 69%). LRMS calculated for C₃₂H₄₄NO₄SiCl: 569; found: 570 (M+H). ¹H NMR (400 MHz, DMSO-d₆) δ ppm: 7.66 (d, J=7.6 Hz, 1H), 7.29 (dd, J=7.5, 1.3 Hz, 1H), 7.25-7.20 (m, 1H), 7.11 (td, J=7.5, 1.2 Hz, 1H), 7.08 (t, J=8.1 Hz, 1H), 6.63 (t, J=2.1 Hz, 1H), 6.61-6.57 (m, 1H), 6.50-6.48 (m, 1H), 6.46-6.42 (m, 2H), 4.47 (t, J=4.9 Hz, 1H), 3.69 (s, 3H), 3.62 (d, J=4.9 Hz, 2H), 3.43 (t, J=5.4 Hz, 2H), 2.48-2.32 (m, 2H), 2.28-1.96 (m, 7H), 0.93-0.78 (m, 11H), 0.00 (s, 3H), −0.02 (s, 3H).

### Example 514B methyl (1r,4r)-2′-{2-({[tert-butyl(dimethyl)silyl]oxy}methyl)-3-[(methanesulfonyl)oxy]propyl}-4-(3-chloroanilino)spiro[cyclohexane-1,1′-indene]-4-carboxylate

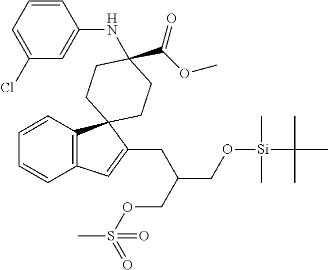 [SMILES: COC(=O)[C@]1(Nc2cccc(Cl)c2)CC[C@]2(CC1)C(CC(CO[Si](C)(C)C(C)(C)C)COS(C)(=O)=O)=Cc1ccccc12]

To a solution of Example 514A (237 mg, 0.42 mmol, 1 eq) in DCM (10 mL), cooled to 0° C., was added DIPEA (0.11 mL, 0.62 mmol, 1.5 eq) followed by MsCl (39 μL, 0.5 mmol, 1.2 eq) and the reaction was slowly allowed to warm to rt and stirred for 2 h. The reaction was partitioned between DCM and water. The organic phase was washed with dilute aq. HCl solution, sat. aq. NaHCO₃ solution, dried (MgSO₄) and concentrated in vacuo. Purification by automated flash chromatography (CombiFlash Rf, 12 g RediSep™ silica cartridge) eluting with a gradient of 0-34% EtOAc in heptane afforded racemate Example 514B as a colourless oil (253 mg, 0.39 mmol, 94%). LRMS calculated for C₃₃H₄₆NO₆SiSCl: 647; found: 648 (M+H). ¹H NMR (400 MHz, DMSO-d₆) δ ppm: 7.67 (d, J=7.5 Hz, 1H), 7.31 (dd, J=7.4, 1.3 Hz, 1H), 7.25 (td, J=7.5, 1.0 Hz, 1H), 7.14 (td, J=7.4, 1.4 Hz, 1H), 7.08 (t, J=8.1 Hz, 1H), 6.64 (t, J=2.1 Hz, 1H), 6.61-6.56 (m, 2H), 6.48-6.42 (m, 2H), 4.24 (d, J=5.4 Hz, 2H), 3.72-3.66 (m, 4H), 3.62 (dd, J=10.1, 5.0 Hz, 1H), 3.16 (s, 3H), 2.48-2.32 (m, 3H), 2.31-2.04 (m, 6H), 0.97-0.83 (m, 2H), 0.81 (s, 9H), 0.02 (s, 3H), 0.00 (s, 3H).

### Example 514C methyl (1r,4r)-4-[(3-chlorophenyl)amino]-2′-[2-(fluoromethyl)-3-hydroxypropyl]spiro[cyclohexane-1,1′-indene]-4-carboxylate

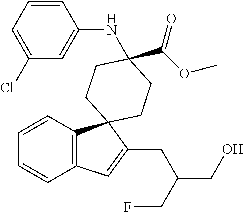 [SMILES: COC(=O)[C@]1(Nc2cccc(Cl)c2)CC[C@]2(CC1)C(CC(CO)CF)=Cc1ccccc12]

A solution of Example 514B (253 mg, 0.39 mmol, 1 eq) in TBAF (1.95 mL, 1 M in THF, 1.95 mmol, 5 eq) was stirred at 80° C. under N₂ for 1.5 h. The reaction was allowed to cool to rt, concentrated in vacuo and then purified by automated flash chromatography (CombiFlash Rf, 12 g RediSep™ silica cartridge) eluting with a gradient of 0-85% EtOAc in heptane to afford racemate Example 514C as a colourless oil (86.4 mg, 0.19 mmol, 48%). LRMS calculated for C₂₆H₂₉NO₃FCl: 457; found: 458 (M+H). ¹H NMR (400 MHz, DMSO-d₆) δ ppm: 7.66 (d, J=7.5 Hz, 1H), 7.31 (dd, J=7.5, 1.3 Hz, 1H), 7.24 (td, J=7.4, 1.0 Hz, 1H), 7.13 (td, J=7.5, 1.3 Hz, 1H), 7.10 (t, J=8.1 Hz, 1H), 6.63 (t, J=2.1 Hz, 1H), 6.62-6.58 (m, 1H), 6.57-6.54 (m, 1H), 6.50-6.46 (m, 1H), 6.45 (s, 1H), 4.74 (t, J=5.1 Hz, 1H), 4.60-4.53 (m, 1H), 4.49-4.41 (m, 1H), 3.70 (s, 3H), 3.54-3.42 (m, 2H), 2.48-2.36 (m, 2H), 2.32-2.06 (m, 7H), 0.95-0.84 (m, 2H).

### Example 514D methyl (1r,4R)-4-(3-chloroanilino)-2′-[2-(fluoromethyl)-3-{[(5R)-5-methyl-5,6,7,8-tetrahydroquinolin-4-yl]oxy}propyl]spiro[cyclohexane-1,1′-indene]-4-carboxylate, diastereoisomer 1

### Example 515A methyl (1r,4R)-4-(3-chloroanilino)-2′-[2-(fluoromethyl)-3-{[(5R)-5-methyl-5,6,7,8-tetrahydroquinolin-4-yl]oxy}propyl]spiro[cyclohexane-1,1′-indene]-4-carboxylate, diastereoisomer 2

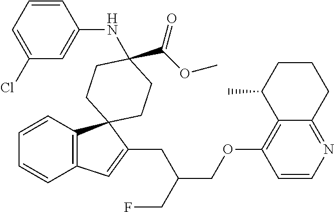 [SMILES: COC(=O)[C@]1(Nc2cccc(Cl)c2)CC[C@]2(CC1)C(CC(CF)COc1ccnc3c1[C@H](C)CCC3)=Cc1ccccc12]

Using General procedure 30a and Example 514C (86.4 mg, 0.19 mmol, 1 eq) as the appropriate indene and Preparation 2a1 (62 mg, 0.38 mmol, 2 eq) as the appropriate alcohol, a mixture of diastereoisomers was obtained as a white powder (46.4 mg, 0.08 mmol, 41%). The diastereoisomers were separated by chiral chromatography. Column: AD, 50×500 mm, 20 μm, Eluents: 30:70 nPrOH/heptane. The diastereoisomer eluting earlier was collected as Example 514D. ¹H NMR (500 MHz, DMSO-d₆) δ ppm: 8.14 (d, 1H), 7.65 (d, 1H), 7.31 (d, 1H), 7.24 (t, 1H), 7.13 (t, 1H), 7.08 (t, 1H), 6.79 (d, 1H), 6.64 (s, 1H), 6.62 (dd, 1H), 6.59 (dd, 1H), 6.45 (s, 1H), 6.45 (dd, 1H), 4.68/4.58 (m+m, 2H), 4.17/4.07 (dd+dd, 2H), 3.68 (s, 3H), 3.05 (m, 1H), 2.75/2.62 (dd+dd, 2H), 2.71 (m, 1H), 2.43 (d, 2H), 2.41-0.8 (m, 8H), 1.79/1.68 (m+m, 2H), 1.58/1.52 (m+m, 2H), 1.1 (d, 3H). HRMS calculated for C₃₆H₄₀ClFN₂O₃: 602.2712; found: 603.2781 (M+H).

The diastereoisomer eluting later was collected as Example 515A. ¹H NMR (500 MHz, DMSO-d₆) δ ppm: 8.16 (d, 1H), 7.67 (d, 1H), 7.31 (d, 1H), 7.24 (t, 1H), 7.14 (t, 1H), 7.08 (t, 1H), 6.83 (d, 1H), 6.65 (s, 1H), 6.63 (dd, 1H), 6.58 (dd, 1H), 6.43 (s, 1H), 6.42 (dd, 1H), 4.68/4.58 (m+m, 2H), 4.15/4.11 (dd+dd, 2H), 3.68 (s, 3H), 3.07 (m, 1H), 2.77/2.68 (dd+dd, 2H), 2.72 (m, 1H), 2.43 (d, 2H), 2.42-0.87 (m, 8H), 1.8/1.74 (m+m, 2H), 1.66/1.59 (m+m, 2H), 1.1 (d, 3H). HRMS calculated for C₃₆H₄₀ClFN₂O₃: 602.2712; found: 603.2767 (M+H).

### Example 514 (1r,4R)-4-(3-chloroanilino)-2′-[2-(fluoromethyl)-3-{[(5R)-5-methyl-5,6,7,8-tetrahydroquinolin-4-yl]oxy}propyl]spiro[cyclohexane-1,1′-indene]-4-carboxylic acid, diastereoisomer 1

and

### Example 515 (1r,4R)-4-(3-chloroanilino)-2′-[2-(fluoromethyl)-3-{[(5R)-5-methyl-5,6,7,8-tetrahydroquinolin-4-yl]oxy}propyl]spiro[cyclohexane-1,1′-indene]-4-carboxylic acid, diastereoisomer 2

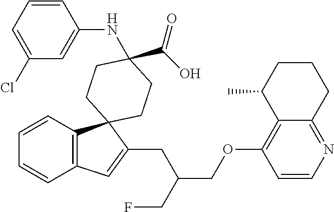 [SMILES: C[C@@H]1CCCc2nccc(OCC(CF)CC3=Cc4ccccc4[C@]34CC[C@@](Nc3cccc(Cl)c3)(C(=O)O)CC4)c21]

Using General procedure 33a and Example 514D as the appropriate ester Example 514 was obtained. ¹H NMR (500 MHz, DMSO-d₆) δ ppm: 12.84 (br s, 1H), 8.14 (d, 1H), 7.66 (d, 1H), 7.3 (d, 1H), 7.23 (t, 1H), 7.13 (t, 1H), 7.07 (t, 1H), 6.8 (d, 1H), 6.63 (s, 1H), 6.63 (dd, 1H), 6.57 (dd, 1H), 6.55 (dd, 1H), 6.36 (br s, 1H), 4.63 (m, 2H), 4.17/4.07 (dd+dd, 2H), 3.06 (m, 1H), 2.75/2.63 (dd+dd, 2H), 2.7 (m, 1H), 2.46-0.79 (m, 8H), 2.43 (d, 2H), 1.78/1.69 (m+m, 2H), 1.58/1.52 (m+m, 2H), 1.11 (d, 3H). HRMS calculated for C₃₅H₃₈N₂O₃FCl: 588.2555; found: 589.2622 (M+H).

Using General procedure 33a and Example 515A as the appropriate ester Example 515 was obtained. ¹H NMR (500 MHz, DMSO-d₆) δ ppm: 12.85 (br s, 1H), 8.15 (d, 1H), 7.68 (d, 1H), 7.31 (d, 1H), 7.24 (t, 1H), 7.13 (t, 1H), 7.06 (t, 1H), 6.8 (d, 1H), 6.64 (s, 1H), 6.63 (dd, 1H), 6.55 (dd, 1H), 6.52 (dd, 1H), 6.33 (br s, 1H), 4.63 (m, 2H), 4.14/4.1 (dd+dd, 2H), 3.07 (m, 1H), 2.77/2.65 (dd+dd, 2H), 2.71 (m, 1H), 2.43 (d, 2H), 2.42-0.84 (m, 8H), 1.81/1.74 (m+m, 2H), 1.66/1.59 (m+m, 2H), 1.1 (d, 3H). HRMS calculated for C₃₅H₃₈N₂O₃FCl: 588.2555; found: 589.2622 (M+H).

### Example 516 and Example 517

### Example 516A methyl (1r,4r)-2′-[2-({[tert-butyl(dimethyl)silyl]oxy}methyl)-3-oxopropyl]-4-(3-chloroanilino)spiro[cyclohexane-1,1′-indene]-4-carboxylate

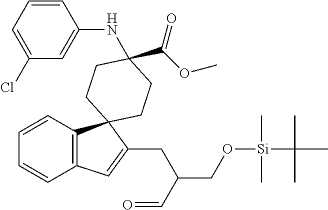 [SMILES: COC(=O)[C@]1(Nc2cccc(Cl)c2)CC[C@]2(CC1)C(CC(C=O)CO[Si](C)(C)C(C)(C)C)=Cc1ccccc12]

To a solution of Example 514A (230 mg, 0.4 mmol, 1 eq) in DCM (6 mL) cooled to 0° C., was added DMP (171 mg, 0.4 mmol, 1 eq) portionwise and the mixture was slowly allowed to warm to rt and stirred for 18 h. The reaction was quenched with 10% aq. Na₂S₂O₃ solution and then extracted with DCM. The organic phase was washed with sat. aq. NaHCO₃ solution, dried (MgSO₄) and concentrated in vacuo. Purification by automated flash chromatography (CombiFlash Rf, 12 g RediSep™ silica cartridge) eluting with a gradient of 0-32% EtOAc in heptane afforded racemate Example 516A as a colourless gum (192.1 mg, 0.34 mmol, 84%). LRMS calculated for C₃₂H₄₂NO₄SiCl: 567; found: 568 (M+H). ¹H NMR (400 MHz, DMSO-d₆) δ ppm: 9.70 (d, J=1.5 Hz, 1H), 7.67 (d, J=7.5 Hz, 1H), 7.28 (dd, J=7.4, 1.4 Hz, 1H), 7.23 (td, J=7.3, 0.9 Hz, 1H), 7.13 (td, J=7.4, 1.5 Hz, 1H), 7.08 (t, J=8.1 Hz, 1H), 6.65 (t, J=2.1 Hz, 1H), 6.61-6.57 (m, 1H), 6.49-6.41 (m, 3H), 4.06 (dd, J=10.3, 3.9 Hz, 1H), 3.86 (dd, J=10.4, 5.0 Hz, 1H), 3.70 (s, 3H), 3.03-2.95 (m, 1H), 2.70-2.61 (m, 1H), 2.49-2.06 (m, 7H), 0.96-0.83 (m, 2H), 0.78 (s, 9H), 0.00 (s, 3H), −0.01 (s, 3H).

### Example 516B methyl (1r,4r)-2′-[2-({[tert-butyl(dimethyl)silyl]oxy}methyl)-3,3-difluoropropyl]-4-(3-chloroanilino)spiro[cyclohexane-1,1′-indene]-4-carboxylate

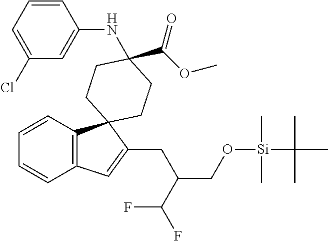 [SMILES: COC(=O)[C@]1(Nc2cccc(Cl)c2)CC[C@]2(CC1)C(CC(CO[Si](C)(C)C(C)(C)C)C(F)F)=Cc1ccccc12]

To a solution of Example 516A (286 mg, 0.5 mmol, 1 eq) in DCM (5 mL) cooled to 0° C., under N₂, was added DAST (133 μL, 1.01 mmol, 2 eq) dropwise and the mixture was slowly allowed to warm to rt and stirred for 18 h. The mixture was cooled to 0° C. and quenched with the careful addition of 2 M aq. NaOH solution. The organics were extracted with DCM, dried (MgSO₄) and concentrated in vacuo. Purification by automated flash chromatography (CombiFlash Rf, 12 g RediSep™ silica cartridge) eluting with a gradient of 0-20% EtOAc in heptane afforded racemate Example 516B as a colourless gum (243 mg, 0.41 mmol, 82%). LRMS calculated for C₃₂H₄₂NO₃F₂SiCl: 598; found: 590 (M+H). ¹H NMR (400 MHz, DMSO-d₆) δ ppm: 7.67 (d, J=7.5 Hz, 1H), 7.30 (dd, J=7.4, 1.4 Hz, 1H), 7.25 (td, J=7.4, 0.9 Hz, 1H), 7.14 (td, J=7.4, 1.4 Hz, 1H), 7.08 (t, J=8.1 Hz, 1H), 6.64 (t, J=2.1 Hz, 1H), 6.62-6.57 (m, 2H), 6.46-6.41 (m, 2H), 6.08 (td, J=56.4, 4.1 Hz, 1H), 3.77-3.63 (m, 5H), 2.50-2.06 (m, 9H), 0.99-0.91 (m, 1H), 0.89-0.70 (m, 1H), 0.77 (s, 9H), 0.01 (s, 3H), −0.05 (s, 3H).

### Example 516C methyl (1r,4r)-4-(3-chloroanilino)-2′-[3,3-difluoro-2-(hydroxymethyl)propyl]spiro[cyclohexane-1,1′-indene]-4-carboxylate

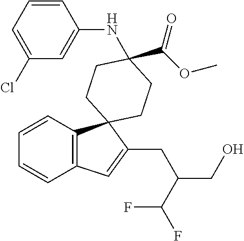 [SMILES: COC(=O)[C@]1(Nc2cccc(Cl)c2)CC[C@]2(CC1)C(CC(CO)C(F)F)=Cc1ccccc12]

Using General procedure 29 and Example 516B (243 mg, 0.41 mmol, 1 eq) as the appropriate silyl derivative, racemate Example 516C was obtained as a colourless gum (160 mg, 0.34 mmol, 82%). LRMS calculated for C₂₆H₂₈NO₃F₂Cl: 475; found: 476 (M+H). ¹H NMR (400 MHz, DMSO-d₆) δ ppm: 7.67 (d, J=7.5 Hz, 1H), 7.31 (dd, J=7.4, 1.3 Hz, 1H), 7.25 (td, J=7.4, 0.9 Hz, 1H), 7.14 (td, J=7.5, 1.3 Hz, 1H), 7.10 (t, J=8.1 Hz, 1H), 6.63 (t, J=2.1 Hz, 1H), 6.62-6.58 (m, 2H), 6.50-6.46 (m, 1H), 6.45 (s, 1H), 6.15 (td, J=56.5, 3.3 Hz, 1H), 4.89 (t, J=5.0 Hz, 1H), 3.70 (s, 3H), 3.60-3.46 (m, 2H), 2.48-2.06 (m, 9H), 0.97-0.84 (m, 2H).

### Example 516D methyl (1r,4R)-4-(3-chloroanilino)-2′-[3,3-difluoro-2-({[(5R)-5-methyl-5,6,7,8-tetrahydroquinolin-4-yl]oxy}methyl)propyl]spiro[cyclohexane-1,1′-indene]-4-carboxylate, diastereoisomer 1

### Example 517A methyl (1r,4R)-4-(3-chloroanilino)-2′-[3,3-difluoro-2-({[(5R)-5-methyl-5,6,7,8-tetrahydroquinolin-4-yl]oxy}methyl)propyl]spiro[cyclohexane-1,1′-indene]-4-carboxylate, diastereoisomer 2

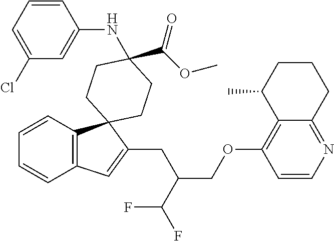 [SMILES: COC(=O)[C@]1(Nc2cccc(Cl)c2)CC[C@]2(CC1)C(CC(COc1ccnc3c1[C@H](C)CCC3)C(F)F)=Cc1ccccc12]

Using General procedure 30a and Example 516C (78 mg, 0.16 mmol, 1 eq) as the appropriate indene and Preparation 2a1 (53.5 mg, 0.33 mmol, 2 eq) as the appropriate alcohol, a mixture of diastereoisomers was obtained as a white foam (58.1 mg, 0.09 mmol, 57%). The diastereoisomers were separated by chiral chromatography. Column: AD, 50×500 mm, 20 μm, Eluents: 15:85 EtOH/heptane. The diastereoisomer eluting earlier was collected as Example 516D. ¹H NMR (500 MHz, DMSO-d₆) δ ppm: 8.14 (d, 1H), 7.67 (d, 1H), 7.31 (d, 1H), 7.24 (t, 1H), 7.14 (t, 1H), 7.07 (t, 1H), 6.77 (d, 1H), 6.67 (s, 1H), 6.63 (dd, 1H), 6.59 (dd, 1H), 6.43 (s, 1H), 6.42 (dd, 1H), 6.3 (td, 1H), 4.22/4.12 (dd+dd, 2H), 3.68 (s, 3H), 3.06 (m, 1H), 2.9 (m, 1H), 2.75/2.65 (dd+dd, 2H), 2.56 (d, 2H), 2.46-0.8 (m, 8H), 1.76/1.73 (m+m, 2H), 1.63/1.57 (m+m, 2H), 1.04 (d, 3H). HRMS calculated for C₃₆H₃₉ClF₂N₂O₃: 620.2617; found: 621.2670 (M+H).

The diastereoisomer eluting later was collected as Example 517A. ¹H NMR (500 MHz, DMSO-d₆) δ ppm: 8.12 (d, 1H), 7.65 (d, 1H), 7.29 (d, 1H), 7.23 (t, 1H), 7.13 (t, 1H), 7.09 (t, 1H), 6.76 (d, 1H), 6.66 (s, 1H), 6.63 (dd, 1H), 6.6 (dd, 1H), 6.46 (s, 1H), 6.45 (dd, 1H), 6.29 (td, 1H), 4.22/4.12 (dd+dd, 2H), 3.68 (s, 3H), 3.01 (m, 1H), 2.92 (m, 1H), 2.73/2.61 (dd+dd, 2H), 2.55 (d, 2H), 2.46-0.73 (m, 8H), 1.76/1.64 (m+m, 2H), 1.48/1.43 (m+m, 2H), 1.06 (d, 3H). HRMS calculated for C₃₆H₃₉ClF₂N₂O₃: 620.2617; found: 621.2694 (M+H).

### Example 516 (1r,4R)-4-(3-chloroanilino)-2′-[3,3-difluoro-2-({[(5R)-5-methyl-5,6,7,8-tetrahydroquinolin-4-yl]oxy}methyl)propyl]spiro[cyclohexane-1,1′-indene]-4-carboxylic acid, diastereoisomer 1

and

### Example 517 (1r,4R)-4-(3-chloroanilino)-2′-[3,3-difluoro-2-({[(5R)-5-methyl-5,6,7,8-tetrahydroquinolin-4-yl]oxy}methyl)propyl]spiro[cyclohexane-1,1′-indene]-4-carboxylic acid, diastereoisomer 2

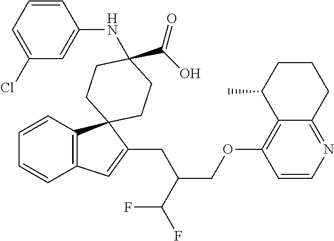 [SMILES: C[C@@H]1CCCc2nccc(OCC(CC3=Cc4ccccc4[C@]34CC[C@@](Nc3cccc(Cl)c3)(C(=O)O)CC4)C(F)F)c21]

Using General procedure 33a and Example 516D as the appropriate ester Example 516 was obtained. ¹H NMR (500 MHz, DMSO-d₆) δ ppm: 12.84 (br s, 1H), 8.13 (d, 1H), 7.68 (d, 1H), 7.3 (d, 1H), 7.24 (t, 1H), 7.14 (t, 1H), 7.06 (t, 1H), 6.78 (d, 1H), 6.67 (s, 1H), 6.63 (dd, 1H), 6.56 (dd, 1H), 6.51 (dd, 1H), 6.33 (br s, 1H), 6.3 (td, 1H), 4.22/4.12 (dd+dd, 2H), 3.06 (m, 1H), 2.91 (m, 1H), 2.75/2.64 (dd+dd, 2H), 2.59/2.53 (dd+dd, 2H), 2.45-0.8 (m, 8H), 1.77/1.73 (m+m, 2H), 1.63/1.58 (m+m, 2H), 1.05 (d, 3H). HRMS calculated for C₃₅H₃₇N₂O₃F₂Cl: 606.2461; found: 607.2538 (M+H).

Using General procedure 33a and Example 517A as the appropriate ester Example 517 was obtained. ¹H NMR (500 MHz, DMSO-d₆) δ ppm: 12.82 (br s, 1H), 8.12 (d, 1H), 7.66 (d, 1H), 7.29 (d, 1H), 7.23 (t, 1H), 7.13 (t, 1H), 7.08 (t, 1H), 6.76 (d, 1H), 6.66 (s, 1H), 6.63 (dd, 1H), 6.58 (dd, 1H), 6.55 (dd, 1H), 6.36 (br s, 1H), 6.29 (td, 1H), 4.22/4.12 (dd+dd, 2H), 3.02 (m, 1H), 2.93 (m, 1H), 2.73/2.59 (dd+dd, 2H), 2.55 (d, 2H), 2.45-0.71 (m, 8H), 1.76/1.64 (m+m, 2H), 1.498/1.43 (m+m, 2H), 1.07 (d, 3H). HRMS calculated for C₃₅H₃₇N₂O₃F₂Cl: 606.2461; found: 607.2527 (M+H).

### Example 518 and Example 519 and Example 520 and Example 521

### Example 518A and Example 520A

### methyl (1r,4r)-4-(3-chloroanilino)-2′-[3-hydroxy-2-(hydroxymethyl)propyl]-2′,3′-dihydrospiro[cyclohexane-1,1′-indene]-4-carboxylate, enantiomer 1

and

### methyl (1r,4r)-4-(3-chloroanilino)-2′-[3-hydroxy-2-(hydroxymethyl)propyl]-2′,3′-dihydrospiro[cyclohexane-1,1′-indene]-4-carboxylate, enantiomer 2

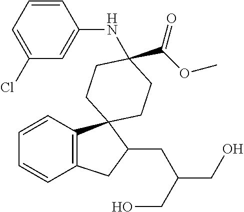

The enantiomers of Preparation 11b were separated by chiral chromatography. Column: AD, 50×500 mm, 20 μm. Eluents: 10:90 EtOH/heptane. The enantiomer eluting earlier was collected as Example 518A. ¹H NMR (500 MHz, DMSO-d₆) δ ppm: 7.39-7.09 (m, 4H), 7.06 (t, 1H), 6.59 (t, 1H), 6.56 (dm, 1H), 6.45 (dm, 1H), 6.30 (s, 1H), 4.36 (t, 1H), 4.33 (t, 1H), 3.64 (s, 3H), 3.49/3.37 (m+m, 2H), 3.46/3.28 (m+m, 2H), 2.96/2.54 (dd+dd, 2H), 2.50-1.30 (m, 8H), 2.13 (m, 1H), 1.55 (m, 1H), 1.33/1.16 (m+m, 2H). HRMS calculated for C₂₆H₃₂NO₄Cl: 457.2020; found: 458.2077 (M+H).

The enantiomer eluting later was collected as Example 520A. HRMS calculated for C₂₆H₃₂NO₄Cl: 457.2020; found: 458.2077 (M+H).

### Example 518B methyl (1r,4r)-2′-[2-({[tert-butyl(dimethyl)silyl]oxy}methyl)-3-hydroxypropyl]-4-(3-chloroanilino)-2′,3′-dihydrospiro[cyclohexane-1,1′-indene]-4-carboxylate, diastereoisomer 1 enantiomer 1 and diastereoisomer 2 enantiomer 1

### Example 520B methyl (1r,4r)-2′-[2-({[tert-butyl(dimethyl)silyl]oxy}methyl)-3-hydroxypropyl]-4-(3-chloroanilino)-2′,3′-dihydrospiro[cyclohexane-1,1′-indene]-4-carboxylate, diastereoisomer 1 enantiomer 2 and diastereoisomer 2 enantiomer 2

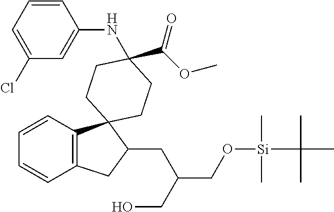

Using General procedure 41a and Example 518A (260 mg, 0.57 mmol, 1 eq) as the appropriate alcohol and TBDMS-Cl (86 mg, 0.57 mmol, 1 eq) as the appropriate silyl chloride, a mixture of 2 diastereoisomers, Example 518B was obtained as a colourless oil (210.6 mg, 0.37 mmol, 65%). LRMS calculated for C₃₂H₄₆NO₄SiCl: 571; found: 572 (M+H). ¹H NMR (400 MHz, DMSO-d₆) δ ppm: 7.40-7.34 (m, 1H), 7.23-7.10 (m, 3H), 7.09-7.03 (m, 1H), 6.62-6.54 (m, 2H), 6.46-6.41 (m, 1H), 6.31/6.28 (s, 1H), 4.42-4.35 (m, 1H), 3.69-3.54 (m, 4H), 3.52-3.24 (m, 3H), 3.01-2.92 (m, 1H), 2.59-2.38 (m, 2H), 2.18-1.68 (m, 6H), 1.65-1.54 (m, 1H), 1.48-1.12 (m, 4H), 0.83/0.80 (s, 9H), 0.02-0.02 (m, 6H).

Using General procedure 41a and Example 520A (185 mg, 0.4 mmol, 1 eq) as the appropriate alcohol and TBDMS-Cl (61 mg, 0.4 mmol, 1 eq) as the appropriate silyl chloride, a mixture of 2 diastereoisomers, Example 520B was obtained as a colourless gum (186.2 mg, 0.33 mmol, 81%). LRMS calculated for C₃₂H₄₆NO₄SiCl: 571; found: 572 (M+H). ¹H NMR (400 MHz, DMSO-d₆) δ ppm: 7.41-7.34 (m, 1H), 7.23-7.10 (m, 3H), 7.09-7.02 (m, 1H), 6.62-6.52 (m, 2H), 6.46-6.41 (m, 1H), 6.32/6.28 (s, 1H), 4.42-4.35 (m, 1H), 3.69-3.54 (m, 4H), 3.52-3.25 (m, 3H), 3.02-2.93 (m, 1H), 2.59-2.38 (m, 2H), 2.18-1.67 (m, 6H), 1.66-1.53 (m, 1H), 1.49-1.11 (m, 4H), 0.83/0.80 (s, 9H), 0.03-0.02 (m, 6H).

### Example 518C methyl (1r,4r)-2′-[2-({[tert-butyl(dimethyl)silyl]oxy}methyl)-3-oxopropyl]-4-(3-chloroanilino)-2′,3′-dihydrospiro[cyclohexane-1,1′-indene]-4-carboxylate, diastereoisomer 1 enantiomer 1 and diastereoisomer 2 enantiomer 1

and

### Example 520C methyl (1r,4r)-2′-[2-({[tert-butyl(dimethyl)silyl]oxy}methyl)-3-oxopropyl]-4-(3-chloroanilino)-2′,3′-dihydrospiro[cyclohexane-1,1′-indene]-4-carboxylate, diastereoisomer 1 enantiomer 2 and diastereoisomer 2 enantiomer 2

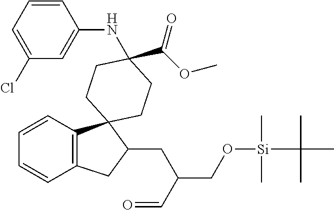

To a solution of Example 518B (210.6 mg, 0.37 mmol, 1 eq) in DCM (8 mL) cooled to 0° C., was added DMP (156 mg, 0.37 mmol, 1 eq) portionwise and the reaction was slowly allowed to warm to rt and stirred under N₂ until no further conversion was observed. The reaction was quenched with 10% aq. Na₂S₂O₃ solution and then extracted with DCM. The organic phase was washed with sat. aq. NaHCO₃ solution, dried (MgSO₄) and concentrated in vacuo. Purification by automated flash chromatography (Combiflash Rf, Silica 4 g RediSep™ cartridge) eluting with a gradient of 0-30% EtOAc in heptane afforded a mixture of 2 diastereoisomers, Example 518C as a brown gum (141.5 mg, 0.25 mmol, 67.43%). LRMS calculated for C₃₂H₄₄NO₄SiCl: 569; found: 570 (M+H). ¹H NMR (400 MHz, DMSO-d₆) δ ppm: 9.69-9.64 (m, 1H), 7.43-7.36 (m, 1H), 7.23-7.11 (m, 3H), 7.06 (t, J=8.1 Hz, 1H), 6.63-6.59 (m, 1H), 6.59-6.55 (m, 1H), 6.46-6.41 (m, 1H), 6.35/6.30 (s, 1H), 4.03-3.82 (m, 2H), 3.65 (s, 3H), 2.99-2.88 (m, 1H), 2.63-2.36 (m, 2H), 2.17-1.69 (m, 7H), 1.68-1.48 (m, 1H), 1.47-1.20 (m, 3H), 0.83/0.78 (s, 9H), 0.04-0.01 (m, 6H).

To a solution of Example 520B (186.2 mg, 0.33 mmol, 1 eq) in DCM (6 mL) cooled to 0° C., was added DMP (138 mg, 0.33 mmol, 1 eq) portionwise and the mixture was slowly allowed to warm to rt and stirred under N₂ for 18 h. The reaction was quenched with 10% aq. Na₂S₂O₃ solution and the organics extracted with DCM. The organic phase was washed with sat. aq. NaHCO₃ solution, dried (MgSO₄) and concentrated in vacuo. Purification by automated flash chromatography (Combiflash Rf, Silica 12 g RediSep™ cartridge) eluting with a gradient of 0-30% EtOAc in heptane afforded a mixture of 2 diastereoisomers, Example 520C as a colourless oil (165.7 mg, 0.29 mmol, 89%). LRMS calculated for C₃₂H₄₄NO₄SiCl: 569; found: 570 (M+H). ¹H NMR (400 MHz, DMSO-d₆) δ ppm: 9.69-9.64 (m, 1H), 7.44-7.36 (m, 1H), 7.23-7.11 (m, 3H), 7.10-7.03 (m, 1H), 6.63-6.52 (m, 2H), 6.47-6.41 (m, 1H), 6.35/6.30 (s, 1H), 4.04-3.81 (m, 2H), 3.65 (s, 3H), 3.00-2.88 (m, 1H), 2.63-2.36 (m, 2H), 2.18-1.69 (m, 7H), 1.68-1.18 (m, 4H), 0.83/0.78 (s, 9H), 0.05-0.01 (m, 6H).

### Example 518D methyl (1r,4r)-2′-[2-({[tert-butyl(dimethyl)silyl]oxy}methyl)-3,3-difluoropropyl]-4-(3-chloroanilino)-2′,3′-dihydrospiro[cyclohexane-1,1′-indene]-4-carboxylate, diastereoisomer 1 enantiomer 1 and diastereoisomer 2 enantiomer 1

and

### Example 520D methyl (1r,4r)-2′-[2-({[tert-butyl(dimethyl)silyl]oxy}methyl)-3,3-difluoropropyl]-4-(3-chloroanilino)-2′,3′-dihydrospiro[cyclohexane-1,1′-indene]-4-carboxylate, diastereoisomer 1 enantiomer 2 and diastereoisomer 2 enantiomer 2

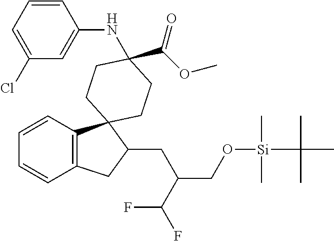

To a solution of Example 518C (141.5 mg, 0.25 mmol, 1 eq) in DCM (5 mL) cooled to 0° C. under N₂ was added DAST (66 μL, 0.5 mmol, 2 eq) dropwise and the reaction was slowly allowed to warm to rt and stirred for 18 h. The mixture was diluted with DCM, cooled to 0° C. and quenched by the careful addition of 2 M aq. NaOH solution. The organic phase was dried (MgSO₄) and concentrated in vacuo. Purification by automated flash chromatography (Combiflash Rf, Silica 12 g RediSep™ cartridge) eluting with a gradient of 0-30% EtOAc in heptane afforded a mixture of 2 diastereoisomers, Example 518D as a colourless oil (96.3 mg, 0.16 mmol, 66%). LRMS calculated for C₃₂H₄₄NO₃F₂SiCl: 591; found: 592 (M+H). ¹H NMR (400 MHz, DMSO-d₆) δ ppm: 7.44-7.35 (m, 1H), 7.24-7.12 (m, 3H), 7.09-7.03 (m, 1H), 6.62-6.55 (m, 2H), 6.47-6.41 (m, 1H), 6.36/6.29 (s, 1H), 6.24-5.82 (m, 1H), 3.77-3.57 (m, 5H), 3.06-2.96 (m, 1H), 2.63-2.38 (m, 2H), 2.21-1.55 (m, 8H), 1.48-1.22 (m, 3H), 0.83/0.80 (s, 9H), 0.04-0.01 (m, 6H).

To a solution of Example 520C (165 mg, 0.29 mmol, 1 eq) in DCM (5 mL) cooled to 0° C. under N₂ was added DAST (76 μL, 0.58 mmol, 2 eq) dropwise and the reaction was slowly allowed to warm to rt and stirred for 18 h. The reaction was diluted with DCM, cooled to 0° C. and quenched by the careful addition of 2 M aq. NaOH solution. The organics were extracted with DCM, dried (MgSO₄) and concentrated in vacuo. Purification by automated flash chromatography (Combiflash Rf, Silica 12 g RediSep™ cartridge) eluting with a gradient of 0-30% EtOAc in heptane afforded a mixture of 2 diastereoisomers, Example 520D as a colourless gum (127.6 mg, 0.22 mmol, 74%). LRMS calculated for C₃₂H₄₄NO₃F₂SiCl: 591; found: 592 (M+H). ¹H NMR (400 MHz, DMSO-d₆) δ ppm: 7.42-7.35 (m, 1H), 7.24-7.11 (m, 3H), 7.10-7.03 (m, 1H), 6.63-6.54 (m, 2H), 6.47-6.41 (m, 1H), 6.36/6.29 (s, 1H), 6.25-5.83 (m, 1H), 3.78-3.58 (m, 5H), 3.06-2.96 (m, 1H), 2.62-2.37 (m, 2H), 2.21-1.55 (m, 8H), 1.49-1.20 (m, 3H), 0.83/0.80 (s, 9H), 0.04-0.01 (m, 6H).

### Example 518E methyl (1r,4r)-4-(3-chloroanilino)-2′-[3,3-difluoro-2-(hydroxymethyl)propyl]-2′,3′-dihydrospiro[cyclohexane-1,1′-indene]-4-carboxylate, diastereoisomer 1 enantiomer 1 and diastereoisomer 2 enantiomer 1

### Example 520E methyl (1r,4r)-4-(3-chloroanilino)-2′-[3,3-difluoro-2-(hydroxymethyl)propyl]-2′,3′-dihydrospiro[cyclohexane-1,1′-indene]-4-carboxylate, diastereoisomer 1 enantiomer 2 and diastereoisomer 2 enantiomer 2

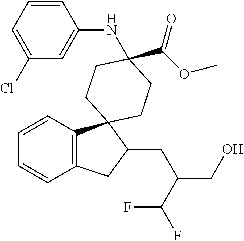

Using General procedure 29 and Example 518D (96.3 mg, 0.16 mmol, 1 eq) as the appropriate silyl derivative, a mixture of 2 diastereoisomers, Example 518E was obtained as an off-white powder (60.1 mg, 0.13 mmol, 77%). LRMS calculated for C₂₆H₃₀NO₃F₂Cl: 477; found: 478 (M+H). ¹H NMR (400 MHz, DMSO-d₆) δ ppm: 7.42-7.34 (m, 1H), 7.25-7.12 (m, 3H), 7.07 (t, J=8.1 Hz, 1H), 6.63-6.55 (m, 2H), 6.49-6.44 (m, 1H), 6.36/6.32 (s, 1H), 6.28-5.90 (m, 1H), 4.86-4.82 (m, 1H), 3.70-3.38 (m, 5H), 3.05-2.95 (m, 1H), 2.62-2.38 (m, 2H), 2.23-1.60 (m, 8H), 1.54-1.18 (m, 3H).

Using General procedure 29 and Example 520D (127.6 mg, 0.22 mmol, 1 eq) as the appropriate silyl derivative, a mixture of 2 diastereoisomers, Example 520E was obtained as an off-white powder (85.2 mg, 0.18 mmol, 83%). LRMS calculated for C₂₆H₃₀NO₃F₂Cl: 477; found: 478 (M+H). ¹H NMR (400 MHz, DMSO-d₆) δ ppm: 7.42-7.34 (m, 1H), 7.25-7.12 (m, 3H), 7.07 (t, J=8.0 Hz, 1H), 6.63-6.55 (m, 2H), 6.49-6.43 (m, 1H), 6.35/6.32 (s, 1H), 6.27-5.90 (m, 1H), 4.87-4.81 (m, 1H), 3.69-3.38 (m, 5H), 3.06-2.96 (m, 1H), 2.63-2.38 (m, 2H), 2.23-1.60 (m, 8H), 1.55-1.19 (m, 3H).

### Example 518F methyl (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylate, diastereoisomer 1

and

### Example 519A methyl (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylate, diastereoisomer 2

and

### Example 520F methyl (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylate, diastereoisomer 3

and

### Example 521A methyl (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylate, diastereoisomer 4

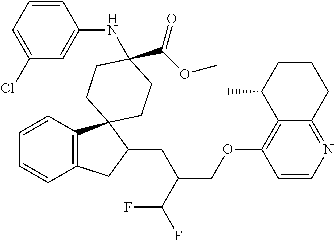

Using General procedure 30a and Example 518E (60 mg, 0.13 mmol, 1 eq) as the appropriate indane and Preparation 2a1 (41 mg, 0.25 mmol, 2 eq) as the appropriate alcohol, a mixture of diastereoisomers was obtained as a white foam (41.5 mg, 0.07 mmol, 53%). The diastereoisomers were separated by chiral chromatography. Column: AD, 50×500 mm, 20 μm, Eluents: 50:50 EtOH/heptane. The diastereoisomer eluting earlier was collected as Example 518F. ¹H NMR (500 MHz, DMSO-d₆) δ ppm: 8.19 (d, 1H), 7.47-7.13 (m, 4H), 7.04 (t, 1H), 6.87 (d, 1H), 6.57 (t, 1H), 6.56 (dm, 1H), 6.38 (dm, 1H), 6.25 (s, 1H), 6.22 (td, 1H), 4.25/4.12 (dd+dd, 2H), 3.63 (s, 3H), 3.07/2.59 (dd+dd, 2H), 3.07 (m, 1H), 2.77/2.66 (dm+m, 2H), 2.49-1.31 (m, 8H), 2.42 (m, 1H), 2.17 (m, 1H), 2.05/1.58 (m+m, 2H), 1.83-1.68 (m, 2H), 1.74/1.51 (m+m, 2H), 1.06 (d, 3H). HRMS calculated for C₃₆H₄₁ClF₂N₂O₃: 622.2774; found: 623.2841 (M+H).

The diastereoisomer eluting later was collected as Example 519A. ¹H NMR (500 MHz, DMSO-d₆) δ ppm: 8.19 (d, 1H), 7.43-7.11 (m, 4H), 7.05 (t, 1H), 6.85 (d, 1H), 6.59 (t, 1H), 6.56 (dm, 1H), 6.45 (dm, 1H), 6.36 (s, 1H), 6.29 (td, 1H), 4.13 (m, 2H), 3.64 (s, 3H), 3.05/2.59 (dd+dd, 2H), 3.05 (m, 1H), 2.78/2.67 (dm+m, 2H), 2.52 (m, 1H), 2.49-1.33 (m, 8H), 2.25 (m, 1H), 1.87/1.46 (m+m, 2H), 1.86-1.68 (m, 2H), 1.67/1.62 (m+m, 2H), 1.02 (d, 3H). HRMS calculated for C₃₆H₄₁ClF₂N₂O₃: 622.2774; found: 623.2833 (M+H).

Using General procedure 30a and Example 520E (85 mg, 0.18 mmol, 1 eq) as the appropriate indane and Preparation 2a1 (58 mg, 0.36 mmol, 2 eq) as the appropriate alcohol, a mixture of diastereoisomers was obtained as a white foam (44.7 mg, 0.07 mmol, 40%). The diastereoisomers were separated by chiral chromatography. Column: AD, 50×500 mm, 20 μm, Eluents: 50:50 EtOH/heptane. The diastereoisomer eluting earlier was collected as Example 520F. ¹H NMR (500 MHz, DMSO-d₆) δ ppm: 8.17 (d, 1H), 7.41-7.11 (m, 4H), 7.05 (t, 1H), 6.8 (d, 1H), 6.61 (t, 1H), 6.56 (dm, 1H), 6.44 (dm, 1H), 6.34 (s, 1H), 6.29 (td, 1H), 4.17/4.04 (dd+dd, 2H), 3.64 (s, 3H), 3.07/2.63 (dd+dd, 2H), 3.03 (m, 1H), 2.77/2.66 (dm+m, 2H), 2.5 (m, 1H), 2.49-1.34 (m, 8H), 2.27 (m, 1H), 1.89/1.4 (m+m, 2H), 1.86-1.69 (m, 2H), 1.63 (m, 2H), 1.12 (d, 3H). HRMS calculated for C₃₆H₄₁ClF₂N₂O₃: 622.2774; found: 623.2831 (M+H).

The diastereoisomer eluting later was collected as Example 521A. ¹H NMR (500 MHz, DMSO-d₆) δ ppm: 8.18 (d, 1H), 7.44-7.12 (m, 4H), 7.06 (t, 1H), 6.8 (d, 1H), 6.58 (t, 1H), 6.57 (dm, 1H), 6.41 (dm, 1H), 6.26 (s, 1H), 6.2 (td, 1H), 4.26/4.14 (dd+dd, 2H), 3.63 (s, 3H), 3.04/2.58 (dd+dd, 2H), 3.04 (m, 1H), 2.74/2.6 (dm+m, 2H), 2.48-1.31 (m, 8H), 2.44 (m, 1H), 2.11 (m, 1H), 1.78/1.49 (m+m, 2H), 1.75/1.64 (m+m, 2H), 1.49/1.43 (m+m, 2H), 1.06 (d, 3H). HRMS calculated for C₃₆H₄₁ClF₂N₂O₃: 622.2774; found: 623.2829 (M+H).

### Example 518 (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylic acid, diastereoisomer 1

and

### Example 519 (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylic acid, diastereoisomer 2

### Example 520 (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylic acid, diastereoisomer 3

and

### Example 521 (1r,4R)-4-(3-chloroanilino)-2′-[3,3-difluoro-2-({[(5R)-5-methyl-5,6,7,8-tetrahydroquinolin-4-yl]oxy}methyl)propyl]-2′,3′-dihydrospiro[cyclohexane-1,1′-indene]-4-carboxylic acid, diastereoisomer 4

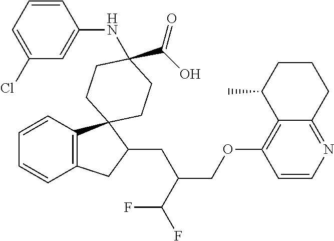

Using General procedure 33a and Example 518F as the appropriate ester Example 518 was obtained. ¹H NMR (500 MHz, DMSO-d₆) δ ppm: 12.74 (br s, 1H), 8.16 (d, 1H), 7.46-7.12 (m, 4H), 7.02 (t, 1H), 6.83 (d, 1H), 6.6 (t, 1H), 6.53 (dm, 1H), 6.47 (dm, 1H), 6.21 (td, 1H), 6.14 (br s, 1H), 4.23/4.1 (dd+dd, 2H), 3.07 (m, 1H), 3.07/2.59 (dd+dd, 2H), 2.76/2.64 (dm+m, 2H), 2.48-1.3 (m, 8H), 2.42 (m, 1H), 2.17 (m, 1H), 1.83-1.67 (m, 2H), 1.74/1.51 (m+m, 2H), 1.63/1.56 (m+m, 2H), 1.06 (d, 3H). HRMS calculated for C₃₅H₃₉N₂O₃F₂Cl: 608.2617; found: 609.2686 (M+H).

Using General procedure 33a and Example 519A as the appropriate ester Example 519 was obtained. ¹H NMR (500 MHz, DMSO-d₆) δ ppm: 12.74 (br s, 1H), 8.17 (d, 1H), 7.41-7.11 (m, 4H), 7.04 (t, 1H), 6.81 (d, 1H), 6.62 (t, 1H), 6.55 (dm, 1H), 6.53 (dm, 1H), 6.28 (td, 1H), 6.26 (br s, 1H), 4.16-4.06 (m, 2H), 3.05/2.59 (dd+dd, 2H), 3.04 (m, 1H), 2.77/2.66 (dm+m, 2H), 2.49 (m, 1H), 2.47-1.34 (m, 8H), 2.25 (m, 1H), 1.87/1.46 (m+m, 2H), 1.85-1.68 (m, 2H), 1.67/1.6 (m+m, 2H), 1.02 (d, 3H). HRMS calculated for C₃₅H₃₉N₂O₃F₂Cl: 608.2617; found: 609.2685 (M+H).

Using General procedure 33a and Example 520F as the appropriate ester Example 520 was obtained. ¹H NMR (500 MHz, DMSO-d₆) δ ppm: 12.72 (br s, 1H), 8.16 (d, 1H), 7.39-7.11 (m, 4H), 7.04 (t, 1H), 6.79 (d, 1H), 6.64 (t, 1H), 6.53 (dm, 1H), 6.53 (dm, 1H), 6.28 (td, 1H), 6.21 (br s, 1H), 4.16/4.02 (dd+dd, 2H), 3.07/2.62 (dd+dd, 2H), 3.03 (m, 1H), 2.77/2.64 (m+m, 2H), 2.5-1.38 (m, 12H), 2.49 (m, 1H), 2.27 (m, 1H), 1.9/1.4 (m+m, 2H), 1.13 (d, 3H). HRMS calculated for C₃₅H₃₉N₂O₃F₂Cl: 608.2617; found: 609.2698 (M+H).

Using General procedure 33a and Example 521A as the appropriate ester Example 521 was obtained. ¹H NMR (500 MHz, DMSO-d₆) δ ppm: 12.74 (br s, 1H), 8.15 (d, 1H), 7.43-7.11 (m, 4H), 7.03 (t, 1H), 6.83 (d, 1H), 6.61 (t, 1H), 6.53 (dm, 1H), 6.5 (dm, 1H), 6.2 (td, 1H), 6.11 (br s, 1H), 4.24/4.12 (dd+dd, 2H), 3.05 (m, 1H), 3.04/2.57 (dd+dd, 2H), 2.73/2.59 (m+m, 2H), 2.46-1.3 (m, 12H), 2.42 (m, 1H), 2.1 (m, 1H), 1.79/1.48 (m+m, 2H), 1.06 (d, 3H). HRMS calculated for C₃₅H₃₉N₂O₃F₂Cl: 608.2617; found: 609.2685 (M+H).

### Example 522 and Example 523 and Example 524 and Example 525

### Example 522A 2-isopropylpropane-1,3-diol

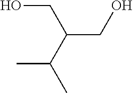

Using General procedure 1 and diethyl 2-isopropylpropanedioate as the appropriate ester, Example 522A was obtained. ¹H NMR (400 MHz, DMSO-d₆) δ ppm: 4.25 (t, 2H), 3.47-3.36 (m, 4H), 1.81-1.72 (m, 1H), 1.32-1.25 (m, 1H), 0.86 (d, 6H).

### Example 522B 1-{[2-(bromomethyl)-3-methylbutoxy]methyl}-4-methoxybenzene

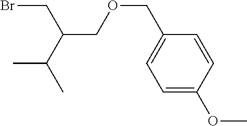 [SMILES: COc1ccc(COCC(CBr)C(C)C)cc1]

Using General procedure 2 and Example 522A as the appropriate diol, an intermediate was obtained which was treated as described in General procedure 3 to obtain Example 522B as a racemate. ¹H NMR (400 MHz, DMSO-d₆) δ ppm: 7.27-7.24 (m, 2H), 6.93-6.89 (m, 2H), 4.42 (d, 1H), 4.37 (d, 1H), 3.74 (s, 3H), 3.71-3.67 (m, 1H), 3.59-3.51 (m, 2H), 3.39-3.35 (m, 1H), 1.92-1.64 (m, 2H), 0.90 (d, 3H), 0.88 (d, 3H). LRMS calculated for C₁₄H₂₁BrO₂: 300.07; found 300.1 (M+).

### Example 522C methyl (1r,4r)-4-(3-chloroanilino)-2′-(2-{[(4-methoxyphenyl)methoxy]methyl}-3-methylbutyl)spiro[cyclohexane-1,1′-indene]-4-carboxylate

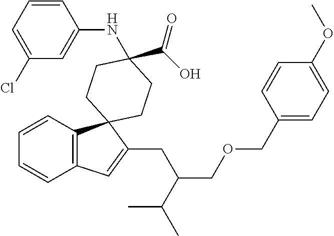 [SMILES: COc1ccc(COCC(CC2=Cc3ccccc3[C@]23CC[C@@](Nc2cccc(Cl)c2)(C(=O)O)CC3)C(C)C)cc1]

Using General procedure 4 and Example 522B as the appropriate bromo compound, an intermediate was obtained, which was used as the appropriate Zn reagent, and treated as described in General procedure 27a, using Preparation 6c as the appropriate 2-bromo-indene derivative to obtain Example 522C as a racemate. ¹H NMR (500 MHz, DMSO-d₆) δ ppm: 7.64 (d, 1H), 7.25 (d, 1H), 7.21 (t, 1H), 7.17 (d, 2H), 7.10 (t, 1H), 7.09 (t, 1H), 6.82 (d, 2H), 6.64 (t, 1H), 6.60 (dd, 1H), 6.47 (dd, 1H), 6.43 (s, 1H), 6.39 (s, 1H), 4.31 (dd, 2H), 3.71 (s, 3H), 3.70 (s, 3H), 3.34 (m, 2H), 2.38/2.21 (m+m, 4H), 2.19 (m, 2H), 2.12/0.83 (m+m, 4H), 1.86 (m, 2H), 0.91/0.88 (d+d, 6H). HRMS calculated for C₃₆H₄₂ClNO₄: 587.2802; found 588.2859 (M+H).

### Example 522D methyl (1r,4r)-4-(3-chloroanilino)-2′-[2-(hydroxymethyl)-3-methylbutyl]spiro[cyclohexane-1,1′-indene]-4-carboxylate, enantiomer 1

and

### Example 524D methyl (1r,4r)-4-(3-chloroanilino)-2′-[2-(hydroxymethyl)-3-methylbutyl]spiro[cyclohexane-1,1′-indene]-4-carboxylate, enantiomer 2

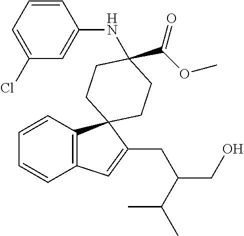 [SMILES: COC(=O)[C@]1(Nc2cccc(Cl)c2)CC[C@]2(CC1)C(CC(CO)C(C)C)=Cc1ccccc12]

Using General procedure 28b and Example 522C as the appropriate PMB derivative, a racemate was obtained. The enantiomers were separated by chiral chromatography. Column: AS-V, 100×500 mm, 20 μm, Eluents: 15:85 EtOH/heptane. The enantiomer eluting earlier was collected as Example 522D. ¹H NMR (500 MHz, DMSO-d₆) δ ppm: 7.64 (dm, 1H), 7.28 (dm, 1H), 7.21 (m, 1H), 7.10 (m, 1H), 7.09 (t, 1H), 6.63 (t, 1H), 6.59 (dm, 1H), 6.46 (s, 1H), 6.46 (m, 1H), 6.43 (s, 1H), 4.37 (t, 1H), 3.70 (s, 3H), 3.37 (m, 2H), 2.45-0.80 (m, 8H), 2.13 (m, 2H), 1.91 (m, 1H), 1.72 (m, 1H), 0.90 (d, 3H), 0.89 (d, 3H). HRMS calculated for C₂₈H₃₄ClNO₃: 467.2227; found 468.2292 (M+H).

The enantiomer eluting later was collected as Example 524D. ¹H NMR (500 MHz, DMSO-d₆) δ ppm: 7.64 (dm, 1H), 7.28 (dm, 1H), 7.21 (m, 1H), 7.10 (m, 1H), 7.09 (t, 1H), 6.63 (t, 1H), 6.59 (dm, 1H), 6.46 (s, 1H), 6.46 (dm, 1H), 6.43 (s, 1H), 4.37 (t, 1H), 3.70 (s, 3H), 3.37 (m, 2H), 2.45-0.80 (m, 8H), 2.13 (m, 2H), 1.91 (m, 1H), 1.72 (m, 1H), 0.90 (d, 3H), 0.89 (d, 3H). HRMS calculated for C₂₈H₃₄ClNO₃: 467.2227; found 468.2293 (M+H).

### Example 522E methyl (1r,4r)-4-(3-chloroanilino)-2′-[2-(hydroxymethyl)-3-methylbutyl]-2′,3′-dihydrospiro[cyclohexane-1,1′-indene]-4-carboxylate, diastereoisomer 1 enantiomer 1

and

### Example 523E methyl (1r,4r)-4-(3-chloroanilino)-2′-[2-(hydroxymethyl)-3-methylbutyl]-2′,3′-dihydrospiro[cyclohexane-1,1′-indene]-4-carboxylate, diastereoisomer 2 enantiomer 1

and

### Example 524E methyl (1r,4r)-4-(3-chloroanilino)-2′-[2-(hydroxymethyl)-3-methylbutyl]-2′,3′-dihydrospiro[cyclohexane-1,1′-indene]-4-carboxylate, diastereoisomer 1 enantiomer 2

and

### Example 525E methyl (1r,4r)-4-(3-chloroanilino)-2′-[2-(hydroxymethyl)-3-methylbutyl]-2′,3′-dihydrospiro[cyclohexane-1,1′-indene]-4-carboxylate, diastereoisomer 2 enantiomer 2

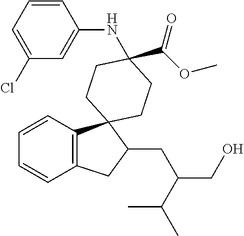

Using General procedure 19 and Example 522D as the appropriate indene, a mixture of diastereoisomers was obtained. They were separated by chiral chromatography. Column: AD, 50×500 mm, 20 μm, Eluents: 25:75 iPrOH/heptane. The diastereoisomer eluting earlier was collected as Example 522E. ¹H NMR (500 MHz, DMSO-d₆) δ ppm: 7.39-7.09 (m, 4H), 7.06 (t, 1H), 6.60 (t, 1H), 6.57 (dm, 1H), 6.45 (dm, 1H), 6.28 (s, 1H), 4.33 (t, 1H), 3.65 (s, 3H), 3.45/3.34 (m+m, 2H), 2.96/2.47 (dd+dd, 2H), 2.48-1.28 (m, 8H), 2.07 (m, 1H), 1.85 (m, 1H), 1.28 (m, 1H), 1.20/1.04 (m+m, 2H), 0.85 (d, 3H), 0.77 (d, 3H). HRMS calculated for C₂₈H₃₆ClNO₃: 469.2384; found 470.2448 (M+H).

The diastereoisomer eluting later was collected as Example 523E. ¹H NMR (500 MHz, DMSO-d₆) δ ppm: 7.42-7.10 (m, 4H), 7.06 (t, 1H), 6.60 (t, 1H), 6.45 (dm, 1H), 6.35 (s, 1H), 5.57 (dm, 1H), 4.30 (t, 1H), 3.65 (s, 3H), 3.41/3.23 (m+m, 2H), 2.93/2.53 (m+m, 2H), 2.49-1.24 (m, 8H), 1.98 (m, 1H), 1.83 (m, 1H), 1.38/1.19 (m+m, 2H), 1.37 (m, 1H), 0.91 (d, 3H), 0.84 (d, 3H). HRMS calculated for C₂₈H₃₆ClNO₃: 469.2384; found 470.2447 (M+H).

Using General procedure 19 and Example 524D as the appropriate indene, a mixture of diastereoisomers was obtained. They were separated by chiral chromatography. Column: AS-V, 100×500 mm, 20 μm, Eluents: 15:85 EtOH/heptane. The diastereoisomer eluting earlier was collected as Example 524E. HRMS calculated for C₂₈H₃₆ClNO₃: 469.2384; found 470.2449 (M+H).

The diastereoisomer eluting later was collected as Example 525E. HRMS calculated for C₂₈H₃₆ClNO₃: 469.2384; found 470.2453 (M+H).

### Example 522 (1r,4r)-4-(3-chloroanilino)-2′-(3-methyl-2-{[(thieno[3,2-b]pyridin-7-yl)oxy]methyl}butyl)-2′,3′-dihydrospiro[cyclohexane-1,1′-indene]-4-carboxylic acid, diastereoisomer 1 enantiomer 1

and

### Example 523 (1r,4r)-4-(3-chloroanilino)-2′-(3-methyl-2-{[(thieno[3,2-b]pyridin-7-yl)oxy]methyl}butyl)-2′,3′-dihydrospiro[cyclohexane-1,1′-indene]-4-carboxylic acid, diastereoisomer 2 enantiomer 1

and

### Example 524 (1r,4r)-4-(3-chloroanilino)-2′-(3-methyl-2-{[(thieno[3,2-b]pyridin-7-yl)oxy]methyl}butyl)-2′,3′-dihydrospiro[cyclohexane-1,1′-indene]-4-carboxylic acid, diastereoisomer 1 enantiomer 2

and

### Example 525 (1r,4r)-4-(3-chloroanilino)-2′-(3-methyl-2-{[(thieno[3,2-b]pyridin-7-yl)oxy]methyl}butyl)-2′,3′-dihydrospiro[cyclohexane-1,1′-indene]-4-carboxylic acid, diastereoisomer 2 enantiomer 2

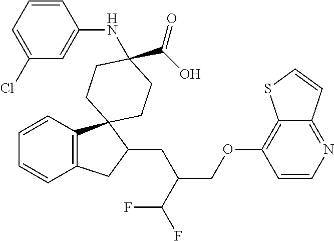

Using General procedure 32 and Example 522E as the appropriate indane and thieno[3,2-b]pyridin-7-ol as the appropriate alcohol, Example 522 was obtained. ¹H NMR (500 MHz, DMSO-d₆) δ ppm: 12.67 (br s, 1H), 8.50 (d, 1H), 7.89 (d, 1H), 7.47 (d, 1H), 7.42-7.10 (m, 4H), 7.04 (t, 1H), 7.02 (d, 1H), 6.59 (m, 1H), 6.55 (dm, 1H), 6.50 (dm, 1H), 6.09 (br s, 1H), 4.27 (d, 2H), 3.05/2.56 (dd+dd, 2H), 2.43-1.26 (m, 8H), 2.16 (m, 1H), 1.90 (m, 1H), 1.87/1.39 (m+m, 2H), 1.83 (m, 1H), 0.95 (d, 6H). HRMS calculated for C₃₄H₃₇N₂O₃SCl: 588.2213; found: 589.2277 (M+H).

Using General procedure 32 and Example 523E as the appropriate indane and thieno[3,2-b]pyridin-7-ol as the appropriate alcohol, Example 523 was obtained. ¹H NMR (500 MHz, DMSO-d₆) δ ppm: 12.69 (br s, 1H), 8.51 (d, 1H), 8.00 (d, 1H), 7.50 (d, 1H), 7.43-7.09 (m, 4H), 7.04 (t, 1H), 7.03 (d, 1H), 6.62 (t, 1H), 6.54 (m, 2H), 6.27 (br s, 1H), 4.28/4.13 (dd+dd, 2H), 3.02/2.62 (dd+dd, 2H), 2.47-1.25 (m, 8H), 2.07 (m, 1H), 1.95 (m, 1H), 1.85 (m, 1H), 1.67/1.34 (m+m, 2H), 1.05/0.96 (d+d, 6H). HRMS calculated for C₃₄H₃₇N₂O₃SCl: 588.2213; found: 589.2281 (M+H).

Using General procedure 32 and Example 524E as the appropriate indane and thieno[3,2-b]pyridin-7-ol as the appropriate alcohol, Example 524 was obtained. HRMS calculated for C₃₄H₃₇N₂O₃SCl: 588.2213; found: 589.2276 (M+H).

Using General procedure 32 and Example 525E as the appropriate indane and thieno[3,2-b]pyridin-7-ol as the appropriate alcohol, Example 525 was obtained. HRMS calculated for C₃₄H₃₇N₂O₃SCl: 588.2213; found: 589.2283 (M+H).

### Example 526 (1r,4r)-4-(3-chloroanilino)-2′-(3-methyl-2-{[(pyridin-3-yl)oxy]methyl}butyl)-2′,3′-dihydrospiro[cyclohexane-1,1′-indene]-4-carboxylic acid, diastereoisomer 1 enantiomer 1

and

### Example 527 (1r,4r)-4-(3-chloroanilino)-2′-(3-methyl-2-{[(pyridin-3-yl)oxy]methyl}butyl)-2′,3′-dihydrospiro[cyclohexane-1,1′-indene]-4-carboxylic acid, diastereoisomer 2 enantiomer 1

### Example 528 (1r,4r)-4-(3-chloroanilino)-2′-(3-methyl-2-{[(pyridin-3-yl)oxy]methyl}butyl)-2′,3′-dihydrospiro[cyclohexane-1,1′-indene]-4-carboxylic acid, diastereoisomer 2 enantiomer 2

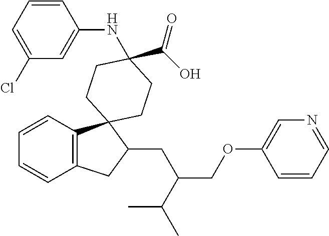

Using General procedure 32 and Example 522E as the appropriate indane and pyridin-3-ol as the appropriate alcohol, Example 526 was obtained. ¹H NMR (500 MHz, DMSO-d₆) δ ppm: 12.69 (br s, 1H), 8.30 (d, 1H), 8.13 (dd, 1H), 7.41-7.09 (m, 6H), 7.05 (t, 1H), 6.62 (t, 1H), 6.54 (m, 2H), 6.16 (br s, 1H), 4.03 (m, 2H), 3.00/2.53 (dd+dd, 2H), 2.44-1.16 (m, 8H), 2.12 (m, 1H), 1.90 (m, 1H), 1.87/1.42 (m+m, 2H), 1.71 (m, 1H), 0.92/0.88 (d+d, 6H). HRMS calculated for C₃₂H₃₇N₂O₃Cl: 532.2493; found: 533.2557 (M+H).

Using General procedure 32 and Example 523E as the appropriate indane and pyridin-3-ol as the appropriate alcohol, Example 527 was obtained. ¹H NMR (500 MHz, DMSO-d₆) δ ppm: 12.69 (br s, 1H), 8.26 (d, 1H), 8.14 (dd, 1H), 7.44-7.09 (m, 6H), 7.05 (t, 1H), 6.62 (t, 1H), 6.54 (dm, 1H), 6.54 (dm, 1H), 6.27 (br s, 1H), 4.05/3.87 (dd+dd, 2H), 2.96/2.58 (dd+dd, 2H), 2.48-1.22 (m, 12H), 2.04 (m, 1H), 1.00 (d, 3H), 0.93 (d, 3H). HRMS calculated for C₃₂H₃₇N₂O₃Cl: 532.2493; found: 533.2363 (M+H).

Using General procedure 32 and Example 525E as the appropriate indane and pyridin-3-ol as the appropriate alcohol, Example 528 was obtained. HRMS calculated for C₃₂H₃₇N₂O₃Cl: 532.2493; found: 533.2566 (M+H).

### Example 529 (1r,4r)-4-(3-chloroanilino)-2′-[3-methyl-2-(phenoxymethyl)butyl]-2′,3′-dihydrospiro[cyclohexane-1,1′-indene]-4-carboxylic acid, diastereoisomer 1 enantiomer 1

and

### Example 530 (1r,4r)-4-(3-chloroanilino)-2′-[3-methyl-2-(phenoxymethyl)butyl]-2′,3′-dihydrospiro[cyclohexane-1,1′-indene]-4-carboxylic acid, diastereoisomer 2 enantiomer 2

and

### Example 531 (1r,4r)-4-(3-chloroanilino)-2′-[3-methyl-2-(phenoxymethyl)butyl]-2′,3′-dihydrospiro[cyclohexane-1,1′-indene]-4-carboxylic acid, diastereoisomer 1 enantiomer 2

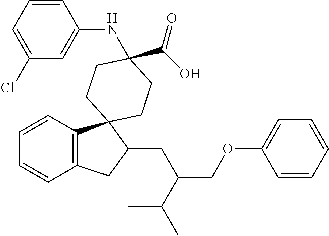

Using General procedure 32 and Example 522E as the appropriate indane and phenol as the appropriate alcohol, Example 529 was obtained. ¹H NMR (500 MHz, DMSO-d₆) δ ppm: 12.65 (br s, 1H), 7.39-7.08 (m, 4H), 7.23 (m, 2H), 7.06 (t, 1H), 6.95/6.93 (dd+dd, 2H), 6.93 (m, 2H), 6.89 (m, 1H), 6.63 (t, 1H), 6.55 (dm, 1H), 6.54 (dm, 1H), 6.16 (br s, 1H), 3.00/2.53 (dd+dd, 2H), 2.46-1.12 (m, 12H), 2.13 (m, 1H), 0.91 (d, 3H), 0.88 (d, 3H). HRMS calculated for C₃₃H₃₈NO₃Cl: 531.2540; found: 532.2623 (M+H).

Using General procedure 32 and Example 525E as the appropriate indane and phenol as the appropriate alcohol, Example 530 was obtained. ¹H NMR (500 MHz, DMSO-d₆) δ ppm: 12.65 (br s, 1H), 7.43-7.10 (m, 4H), 7.26 (m, 2H), 7.05 (t, 1H), 6.91 (m, 2H), 6.90 (m, 1H), 6.62 (t, 1H), 6.54 (dm, 1H), 6.54 (dm, 1H), 6.27 (br s, 1H), 3.96/3.79 (dd+dd, 2H), 2.96/2.58 (dd+dd, 2H), 2.50-1.20 (m, 12H), 2.04 (m, 1H), 0.99 (d, 3H), 0.93 (d, 3H). HRMS calculated for C₃₃H₃₈NO₃Cl: 531.2540; found: 532.2614 (M+H).

Using General procedure 32 and Example 524E as the appropriate indane and phenol as the appropriate alcohol, Example 531 was obtained. HRMS calculated for C₃₃H₃₈NO₃Cl: 531.2540; found: 532.2619 (M+H).

### Example 532 (1r,4r)-4-(3-chloroanilino)-2′-(3-methyl-2-{[(pyridin-4-yl)oxy]methyl}butyl)-2′,3′-dihydrospiro[cyclohexane-1,1′-indene]-4-carboxylic acid, diastereoisomer 2 enantiomer 2

and

### Example 533 (1r,4r)-4-(3-chloroanilino)-2′-(3-methyl-2-{[(pyridin-4-yl)oxy]methyl}butyl)-2′,3′-dihydrospiro[cyclohexane-1,1′-indene]-4-carboxylic acid, diastereoisomer 1 enantiomer 1

and

### Example 534 (1r,4r)-4-(3-chloroanilino)-2′-(3-methyl-2-{[(pyridin-4-yl)oxy]methyl}butyl)-2′,3′-dihydrospiro[cyclohexane-1,1′-indene]-4-carboxylic acid, diastereoisomer 2 enantiomer 1

and

### Example 535 (1r,4r)-4-(3-chloroanilino)-2′-(3-methyl-2-{[(pyridin-4-yl)oxy]methyl}butyl)-2′,3′-dihydrospiro[cyclohexane-1,1′-indene]-4-carboxylic acid, diastereoisomer 1 enantiomer 2

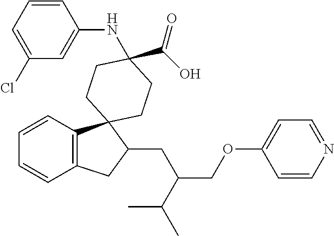

Using General procedure 32 and Example 525E as the appropriate indane and pyridin-4-ol as the appropriate alcohol, Example 532 was obtained. ¹H NMR (500 MHz, DMSO-d₆) δ ppm: 12.65 (br s, 1H), 8.35 (m, 2H), 7.42-7.10 (m, 4H), 7.06 (t, 1H), 6.94 (m, 2H), 6.62 (t, 1H), 6.55 (dm, 1H), 6.54 (dm, 1H), 6.27 (br s, 1H), 4.06/3.89 (dd+dd, 2H), 2.96/2.57 (dd+dd, 2H), 2.49-1.20 (m, 13H), 0.99 (d, 3H), 0.93 (d, 3H). HRMS calculated for C₃₂H₃₇N₂O₃Cl: 532.2493; found: 533.2567 (M+H).

Using General procedure 32 and Example 522E as the appropriate indane and pyridin-4-ol as the appropriate alcohol Example 533 was obtained. ¹H NMR (500 MHz, DMSO-d₆) δ ppm: 12.71 (br s, 1H), 8.32 (d, 2H), 7.35-7.14 (m, 4H), 7.06 (t, 1H), 6.96 (d, 2H), 6.63 (dd, 1H), 6.55 (dm, 1H), 6.54 (dm, 1H), 6.18 (br s, 1H), 4.04 (d, 2H), 3.01/2.53 (m+m, 2H), 2.4-1.22 (m, 12H), 2.12 (m, 1H), 0.91 (d, 3H), 0.88 (d, 3H). HRMS calculated for C₃₂H₃₇N₂O₃Cl: 532.2493; found: 533.2562 (M+H).

Using General procedure 32 and Example 523E as the appropriate indane and pyridin-4-ol as the appropriate alcohol, Example 534 was obtained. HRMS calculated for C₃₂H₃₇N₂O₃Cl: 532.2493; found: 533.2566 (M+H).

Using General procedure 32 and Example 524E as the appropriate indane and pyridin-4-ol as the appropriate alcohol, Example 535 was obtained. ¹H NMR (500 MHz, DMSO-d₆) δ ppm: 12.70 (br s, 1H), 8.32 (m, 2H), 7.37-7.09 (m, 4H), 7.06 (t, 1H), 6.95 (m, 2H), 6.63 (t, 1H), 6.55 (dm, 1H), 6.54 (dm, 1H), 6.18 (br s, 1H), 4.04 (d, 2H), 3.00/2.53 (dd+dd, 2H), 2.47-1.13 (m, 12H), 2.12 (m, 1H), 0.91 (d, 3H), 0.88 (d, 3H). HRMS calculated for C₃₂H₃₇N₂O₃Cl: 532.2493; found: 533.2580 (M+H).

### Example 536 and Example 537 and Example 538 and Example 539

### Example 536A 2-ethylpropane-1,3-diol

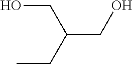

Using General procedure 1 and diethyl 2-ethylpropanedioate as the appropriate ester, Example 536A was obtained. ¹H NMR (400 MHz, DMSO-d₆) δ ppm: 4.28 (t, 2H), 3.41-3.32 (m, 4H), 1.39-1.33 (m, 1H), 1.30-1.23 (m, 2H), 0.86 (t, 3H).

### Example 536B 1-{[2-(bromomethyl)butoxy]methyl}-4-methoxybenzene

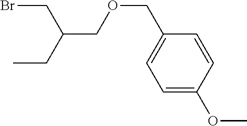 [SMILES: CCC(CBr)COCc1ccc(OC)cc1]

Using General procedure 2 and Example 536A as the appropriate diol, an intermediate was obtained which was treated as described in General procedure 3 to obtain Example 536B as a racemate. ¹H NMR (400 MHz, DMSO-d₆) δ ppm: 7.30-7.23 (m, 2H), 6.95-6.89 (m, 2H), 4.44 (d, 1H), 4.38 (d, 1H), 3.75 (s, 3H), 3.65-3.56 (m, 2H), 3.41-3.32 (m, 2H), 1.87-1.78 (m, 1H), 1.41-1.29 (m, 2H), 0.87 (t, 3H). LRMS calculated for C₁₃H₁₉BrO₂: 286.06; found 286.1 (M+).

### Example 536C methyl (1r,4r)-4-(3-chloroanilino)-2′-(2-{[(4-methoxyphenyl)methoxy]methyl}butyl)spiro[cyclohexane-1,1′-indene]-4-carboxylate

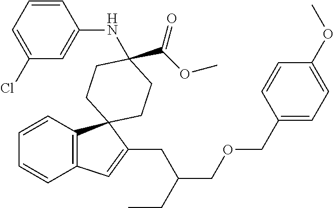 [SMILES: CCC(COCc1ccc(OC)cc1)CC1=Cc2ccccc2[C@]12CC[C@@](Nc1cccc(Cl)c1)(C(=O)OC)CC2]

Using General procedure 4 and Example 536B as the appropriate bromo compound, an intermediate was obtained, which was used as the appropriate Zn reagent, and treated as described in General procedure 27a, using Preparation 6c as the appropriate 2-bromo-indene derivative to obtain Example 536C as a racemate. ¹H NMR (500 MHz, DMSO-d₆) δ ppm: 7.64 (d, 1H), 7.26 (d, 1H), 7.21 (t, 1H), 7.19 (d, 2H), 7.11 (t, 1H), 7.09 (t, 1H), 6.83 (d, 2H), 6.64 (t, 1H), 6.6 (dd, 1H), 6.47 (dd, 1H), 6.43 (s, 1H), 6.41 (s, 1H), 4.35 (dd, 2H), 3.72 (s, 3H), 3.69 (s, 3H), 3.35 (d, 2H), 2.38/2.21 (m+m, 4H), 2.27/2.12 (m+m, 2H), 2.12/0.85 (m+m, 4H), 1.92 (m, 1H), 1.41 (m, 2H), 0.87 (t, 3H). HRMS calculated for C₃₅H₄₀ClNO₄: 573.2646; found 574.2718 (M+H).

### Example 536D methyl (1r,4S)-4-(3-chloroanilino)-2′-[(2S)-2-(hydroxymethyl)butyl]spiro[cyclohexane-1,1′-indene]-4-carboxylate

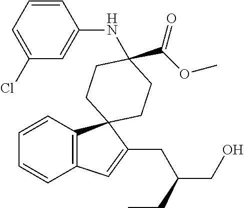 [SMILES: CC[C@H](CO)CC1=Cc2ccccc2[C@]12CC[C@@](Nc1cccc(Cl)c1)(C(=O)OC)CC2]

and

### Example 538D methyl (1r,4R)-4-(3-chloroanilino)-2′-[(2R)-2-(hydroxymethyl)butyl]spiro[cyclohexane-1,1′-indene]-4-carboxylate

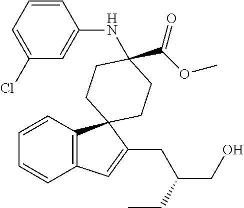 [SMILES: CC[C@@H](CO)CC1=Cc2ccccc2[C@]12CC[C@@](Nc1cccc(Cl)c1)(C(=O)OC)CC2]

Using General procedure 28b and Example 536C as the appropriate PMB derivative, a racemate was obtained. They were separated by chiral chromatography. Column: AS-V, 100×500 mm, 20 μm, Eluents: 15:85 EtOH/heptane. The enantiomer eluting earlier was collected as Example 536D. ¹H NMR (500 MHz, DMSO-d₆) δ ppm: 7.64 (d, 1H), 7.28 (d, 1H), 7.22 (t, 1H), 7.10 (t, 1H), 7.09 (t, 1H), 6.63 (t, 1H), 6.59 (dd, 1H), 6.47 (dd, 1H), 6.46 (s, 1H), 6.43 (s, 1H), 4.44 (t, 1H), 3.69 (s, 3H), 3.38 (m, 2H), 2.40/2.23 (m+m, 4H), 2.28/2.06 (m+m, 2H), 2.13/0.88 (m+m, 4H), 1.76 (m, 1H), 1.40/1.33 (m+m, 2H), 0.88 (t, 3H). HRMS calculated for C₂₇H₃₂ClNO₃: 453.2071; found 454.2142 (M+H).

The enantiomer eluting later was collected as Example 538D. HRMS calculated for C₂₇H₃₂ClNO₃: 453.2071; found 454.2145 (M+H).

### Example 537E methyl (1r,2′S,4S)-4-(3-chloroanilino)-2′-[(2S)-2-(hydroxymethyl)butyl]-2′,3′-dihydrospiro[cyclohexane-1,1′-indene]-4-carboxylate

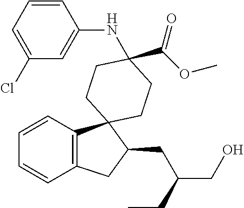 [SMILES: CC[C@H](CO)C[C@H]1Cc2ccccc2[C@]12CC[C@@](Nc1cccc(Cl)c1)(C(=O)OC)CC2]

and

### Example 536E methyl (1r,2′R,4R)-4-(3-chloroanilino)-2′-[(2S)-2-(hydroxymethyl)butyl]-2′,3′-dihydrospiro[cyclohexane-1,1′-indene]-4-carboxylate

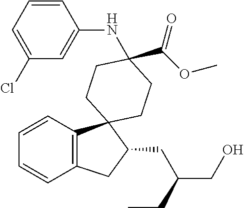 [SMILES: CC[C@H](CO)C[C@@H]1Cc2ccccc2[C@]12CC[C@@](Nc1cccc(Cl)c1)(C(=O)OC)CC2]

Using General procedure 19 and Example 536D as the appropriate indene, a mixture of diastereoisomers was obtained. They were separated by chiral chromatography. Column: AD, 50×500 mm, 20 μm, Eluents: 25:75 iPrOH/heptane. The diastereoisomer eluting earlier was collected as Example 537E. ¹H NMR (500 MHz, DMSO-d₆) δ ppm: 7.39-7.10 (m, 4H), 7.06 (t, 1H), 6.59 (t, 1H), 6.56 (dm, 1H), 6.45 (dm, 1H), 6.29 (s, 1H), 4.33 (t, 1H), 3.64 (s, 3H), 3.44/3.20 (m+m, 2H), 2.95/2.50 (dd+dd, 2H), 2.50-1.27 (m, 8H), 2.09 (m, 1H), 1.48/1.15 (m+m, 2H), 1.36/1.08 (m+m, 2H), 1.36 (m, 1H), 0.84 (t, 3H). HRMS calculated for C₂₇H₃₄ClNO₃: 455.2227; found 456.2301 (M+H).

The diastereoisomer eluting later was collected as Example 536E. ¹H NMR (500 MHz, DMSO-d₆) δ ppm: 7.41-7.10 (m, 4H), 7.06 (t, 1H), 6.59 (t, 1H), 6.56 (dm, 1H), 6.45 (dm, 1H), 6.33 (s, 1H), 4.37 (t, 1H), 3.64 (s, 3H), 3.36/3.25 (m+m, 2H), 2.93/2.52 (dd+dd, 2H), 2.49-1.28 (m, 8H), 2.04 (m, 1H), 1.42 (m, 1H), 1.41/1.27 (m+m, 2H), 1.26 (m, 2H), 0.85 (t, 3H). HRMS calculated for C₂₇H₃₄ClNO₃: 455.2227; found 456.2296 (M+H).

### Example 539E methyl (1r,2′R,4R)-4-(3-chloroanilino)-2′-[(2R)-2-(hydroxymethyl)butyl]-2′,3′-dihydrospiro[cyclohexane-1,1′-indene]-4-carboxylate

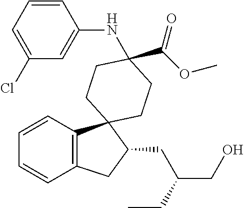 [SMILES: CC[C@@H](CO)C[C@@H]1Cc2ccccc2[C@]12CC[C@@](Nc1cccc(Cl)c1)(C(=O)OC)CC2]

and

### Example 538E methyl (1r,2′S,4S)-4-(3-chloroanilino)-2′-[(2R)-2-(hydroxymethyl)butyl]-2′,3′-dihydrospiro[cyclohexane-1,1′-indene]-4-carboxylate

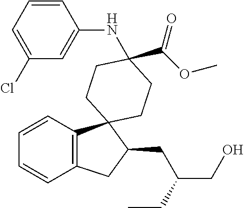 [SMILES: CC[C@@H](CO)C[C@H]1Cc2ccccc2[C@]12CC[C@@](Nc1cccc(Cl)c1)(C(=O)OC)CC2]

Using General procedure 19 and Example 538D as the appropriate indene, a mixture of diastereoisomers was obtained. They were separated by chiral chromatography. Column: AD, 50×500 mm, 20 μm, Eluents: 10:90 EtOH/heptane. The diastereoisomer eluting earlier was collected as Example 539E. HRMS calculated for C₂₇H₃₄ClNO₃: 455.2227; found 456.2299 (M+H).

The diastereoisomer eluting later was collected as Example 538E. HRMS calculated for C₂₇H₃₄ClNO₃: 455.2227; found 456.2296 (M+H).

### Example 536 (1r,2′R,4R)-4-(3-chloroanilino)-2′-[(2S)-2-{[(thieno[3,2-b]pyridin-7-yl)oxy]methyl}butyl]-2′,3′-dihydrospiro[cyclohexane-1,1′-indene]-4-carboxylic acid

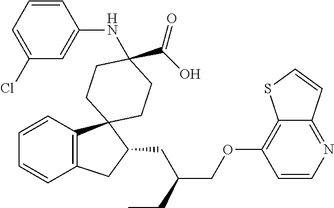 [SMILES: CC[C@H](COc1ccnc2ccsc12)C[C@@H]1Cc2ccccc2[C@]12CC[C@@](Nc1cccc(Cl)c1)(C(=O)O)CC2]

Using General procedure 32 and Example 536E as the appropriate indane and thieno[3,2-b]pyridin-7-ol as the appropriate alcohol, Example 536 was obtained. ¹H NMR (500 MHz, DMSO-d₆) δ ppm: 12.70 (br s, 1H), 8.51 (d, 1H), 8.00 (d, 1H), 7.50 (d, 1H), 7.42-7.09 (m, 4H), 7.04 (t, 1H), 7.01 (d, 1H), 6.62 (t, 1H), 6.54 (dm, 1H), 6.53 (dm, 1H), 6.25 (br s, 1H), 4.23/4.17 (dd+dd, 2H), 3.01/2.58 (dd+dd, 2H), 2.50-1.28 (m, 13H), 2.11 (m, 1H), 0.97 (t, 3H). HRMS calculated for C₃₃H₃₅N₂O₃SCl: 574.2057; found: 575.2132 (M+H).

### Example 537 (1r,2′S,4S)-4-(3-chloroanilino)-2′-[(2S)-2-{[(thieno[3,2-b]pyridin-7-yl)oxy]methyl}butyl]-2′,3′-dihydrospiro[cyclohexane-1,1′-indene]-4-carboxylic acid

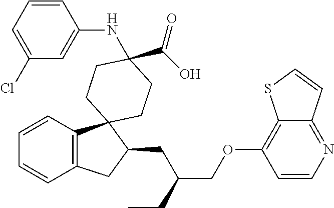 [SMILES: CC[C@H](COc1ccnc2ccsc12)C[C@H]1Cc2ccccc2[C@]12CC[C@@](Nc1cccc(Cl)c1)(C(=O)O)CC2]

Using General procedure 32 and Example 537E as the appropriate indane and thieno[3,2-b]pyridin-7-ol as the appropriate alcohol, Example 537 was obtained. ¹H NMR (500 MHz, DMSO-d₆) δ ppm: 12.66 (br s, 1H), 8.50 (d, 1H), 7.88 (d, 1H), 7.47 (d, 1H), 7.42-7.09 (m, 4H), 7.04 (t, 1H), 7.01 (d, 1H), 6.59 (t, 1H), 6.54 (dm, 1H), 6.50 (dm, 1H), 6.09 (br s, 1H), 4.26/4.23 (dd+dd, 2H), 3.05/2.58 (dd+dd, 2H), 2.46-1.27 (m, 13H), 2.16 (m, 1H), 0.95 (t, 3H). HRMS calculated for C₃₃H₃₅N₂O₃SCl: 574.2057; found: 575.2125 (M+H).

### Example 538 (1r,2′S,4S)-4-(3-chloroanilino)-2′-[(2R)-2-{[(thieno[3,2-b]pyridin-7-yl)oxy]methyl}butyl]-2′,3′-dihydrospiro[cyclohexane-1,1′-indene]-4-carboxylic acid

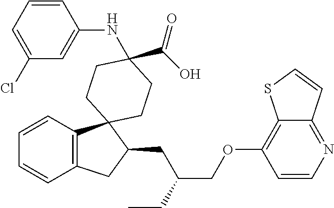 [SMILES: CC[C@@H](COc1ccnc2ccsc12)C[C@H]1Cc2ccccc2[C@]12CC[C@@](Nc1cccc(Cl)c1)(C(=O)O)CC2]

Using General procedure 32 and Example 538E as the appropriate indane and thieno[3,2-b]pyridin-7-ol as the appropriate alcohol, Example 538 was obtained. HRMS calculated for C₃₃H₃₅N₂O₃SCl: 574.2057; found: 575.2118 (M+H).

### Example 539 (1r,2′R,4R)-4-(3-chloroanilino)-2′-[(2R)-2-{[(thieno[3,2-b]pyridin-7-yl)oxy]methyl}butyl]-2′,3′-dihydrospiro[cyclohexane-1,1′-indene]-4-carboxylic acid

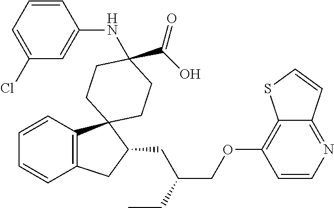 [SMILES: CC[C@@H](COc1ccnc2ccsc12)C[C@@H]1Cc2ccccc2[C@]12CC[C@@](Nc1cccc(Cl)c1)(C(=O)O)CC2]

Using General procedure 32 and Example 539E as the appropriate indane and thieno[3,2-b]pyridin-7-ol as the appropriate alcohol, Example 539 was obtained. HRMS calculated for C₃₃H₃₅N₂O₃SCl: 574.2057; found: 575.2120 (M+H).

### Example 540 (1r,2′S,4S)-4-(3-chloroanilino)-2′-[(2S)-2-{[(pyridin-3-yl)oxy]methyl}butyl]-2′,3′-dihydrospiro[cyclohexane-1,1′-indene]-4-carboxylic acid

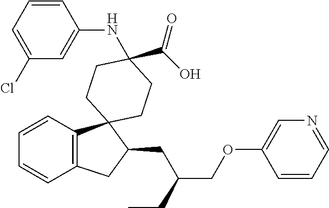 [SMILES: CC[C@H](COc1cccnc1)C[C@H]1Cc2ccccc2[C@]12CC[C@@](Nc1cccc(Cl)c1)(C(=O)O)CC2]

Using General procedure 32 and Example 537E as the appropriate indane and pyridin-3-ol as the appropriate alcohol, Example 540 was obtained. ¹H NMR (500 MHz, DMSO-d₆) δ ppm: 12.67 (br s, 1H), 8.30 (d, 1H), 8.13 (dd, 1H), 7.40-7.09 (m, 4H), 7.38 (dm, 1H), 7.27 (m, 1H), 7.05 (t, 1H), 6.61 (t, 1H), 6.54 (dm, 1H), 6.53 (dm, 1H), 6.18 (br s, 1H), 4.04/3.97 (dd+dd, 2H), 3.00/2.56 (dd+d, 2H), 2.47-1.29 (m, 8H), 2.12 (m, 1H), 1.78 (m, 1H), 1.59/1.26 (m+m, 2H), 1.57/1.36 (m+m, 2H), 0.91 (t, 3H). HRMS calculated for C₃₁H₃₅N₂O₃Cl: 518.2336; found: 519.2398 (M+H).

### Example 541 (1r,2′S,4S)-4-(3-chloroanilino)-2′-[(2S)-2-(phenoxymethyl)butyl]-2′,3′-dihydrospiro[cyclohexane-1,1′-indene]-4-carboxylic acid

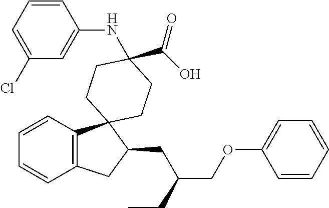 [SMILES: CC[C@H](COc1ccccc1)C[C@H]1Cc2ccccc2[C@]12CC[C@@](Nc1cccc(Cl)c1)(C(=O)O)CC2]

Using General procedure 32 and Example 537E as the appropriate indane and phenol as the appropriate alcohol, Example 541 was obtained. ¹H NMR (500 MHz, DMSO-d₆) δ ppm: 12.67 (br s, 1H), 7.39-7.09 (m, 4H), 7.23 (m, 2H), 7.05 (t, 1H), 6.93 (m, 2H), 6.89 (m, 1H), 6.62 (t, 1H), 6.54 (dm, 1H), 6.54 (dm, 1H), 6.17 (br s, 1H), 3.95/3.88 (dd+dd, 2H), 2.99/2.56 (dd+dd, 2H), 2.46-1.18 (m, 10H), 2.13 (m, 1H), 1.75 (m, 1H), 1.56/1.35 (m+m, 2H), 0.90 (t, 3H). HRMS calculated for C₃₂H₃₆NO₃Cl: 517.2383; found: 518.2460 (M+H).

### Example 542 (1r,2′S,4S)-4-(3-chloroanilino)-2′-[(2S)-2-(phenoxymethyl)butyl]-2′,3′-dihydrospiro[cyclohexane-1,1′-indene]-4-carboxylic acid

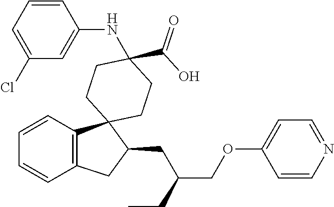 [SMILES: CC[C@H](COc1ccncc1)C[C@H]1Cc2ccccc2[C@]12CC[C@@](Nc1cccc(Cl)c1)(C(=O)O)CC2]

Using General procedure 32 and Example 537E as the appropriate indane and pyridin-4-ol as the appropriate alcohol, Example 542 was obtained. ¹H NMR (500 MHz, DMSO-d₆) δ ppm: 12.59 (br s, 1H), 8.32 (m, 2H), 7.38-7.10 (m, 4H), 7.05 (t, 1H), 6.95 (m, 2H), 6.62 (t, 1H), 6.55 (dm, 1H), 6.54 (dm, 1H), 6.19 (br s, 1H), 4.03/3.99 (dd+dd, 2H), 3.00/2.56 (dd+dd, 2H), 2.46-1.17 (m, 10H), 2.11 (m, 1H), 1.78 (m, 1H), 1.54/1.35 (m+m, 2H), 0.90 (t, 3H). HRMS calculated for C₃₁H₃₅N₂O₃Cl: 518.2336; found: 519.2401 (M+H).

### Example 543 (1r,2′R,4R)-4-(3-chloroanilino)-2′-[(2S)-2-{[(pyridin-4-yl)oxy]methyl}butyl]-2′,3′-dihydrospiro[cyclohexane-1,1′-indene]-4-carboxylic acid

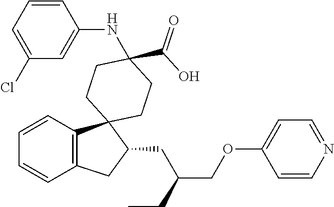 [SMILES: CC[C@H](COc1ccncc1)C[C@@H]1Cc2ccccc2[C@]12CC[C@@](Nc1cccc(Cl)c1)(C(=O)O)CC2]

Using General procedure 32 and Example 536E as the appropriate indane and pyridin-4-ol as the appropriate alcohol, Example 543 was obtained. ¹H NMR (500 MHz, DMSO-d₆) δ ppm: 12.70 (br s, 1H), 8.35 (dm, 2H), 7.38-7.12 (m, 4H), 7.04 (t, 1H), 6.94 (dm, 2H), 6.63 (t, 1H), 6.54 (dd, 1H), 6.53 (dd, 1H), 6.24 (br s, 1H), 4/3.93 (dd+dd, 2H), 2.97/2.55 (dd+dd, 2H), 2.43-1.31 (m, 10H), 2.07 (m, 1H), 1.84 (m, 1H), 1.55/1.43 (m+m, 2H), 0.93 (t, 3H). HRMS calculated for C₃₁H₃₅N₂O₃Cl: 518.2336; found: 519.2405 (M+H).

### Example 544 (1r,2′S,4S)-4-(3-chloroanilino)-2′-[(2R)-2-{[(pyridin-4-yl)oxy]methyl}butyl]-2′,3′-dihydrospiro[cyclohexane-1,1′-indene]-4-carboxylic acid

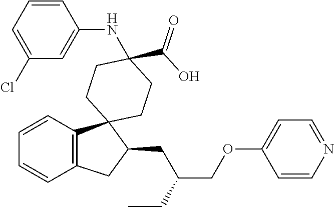 [SMILES: CC[C@@H](COc1ccncc1)C[C@H]1Cc2ccccc2[C@]12CC[C@@](Nc1cccc(Cl)c1)(C(=O)O)CC2]

Using General procedure 32 and Example 538E as the appropriate indane and pyridin-4-ol as the appropriate alcohol, Example 544 was obtained. HRMS calculated for C₃₁H₃₅N₂O₃Cl: 518.2336; found: 519.2399 (M+H).

### Example 545 and Example 546 and Example 547

### Example 545A 3-[(4-methoxyphenyl)methoxy]-2-phenylpropan-1-ol

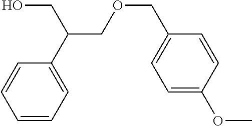 [SMILES: COc1ccc(COCC(CO)c2ccccc2)cc1]

Using General procedure 2 and 2-phenylpropane-1,3-diol as the appropriate diol, Example 545A was obtained as a racemate. ¹H NMR (400 MHz, DMSO-d₆) δ ppm: 7.30-7.16 (m, 7H), 6.89-6.87 (m, 2H), 4.63 (br s, 1H), 4.36 (s, 2H), 3.74 (s, 3H), 3.72-3.56 (m, 4H), 2.99-2.96 (m, 1H).

### Example 545B 1-[(3-bromo-2-phenylpropoxy)methyl]-4-methoxybenzene

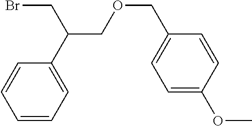 [SMILES: COc1ccc(COCC(CBr)c2ccccc2)cc1]

Using General procedure 3 and Example 545A as the appropriate alcohol, Example 545B was obtained as a racemate. ¹H NMR (400 MHz, DMSO-d₆) δ ppm: 7.38-7.23 (m, 5H), 7.20 (d, 2H), 6.89 (d, 2H), 4.41 (s, 2H), 3.84/3.77 (dd, 2H), 3.70 (s, 3H), 3.65 (dd, 2H), 3.28 (m, 1H). HRMS calculated for C₁₇H₁₉BrO₂: 334.0569; found 334.05519 (M+).

### Example 545C methyl (1r,4r)-4-(3-chloroanilino)-2′-{3-[(4-methoxyphenyl)methoxy]-2-phenylpropyl}spiro[cyclohexane-1,1′-indene]-4-carboxylate

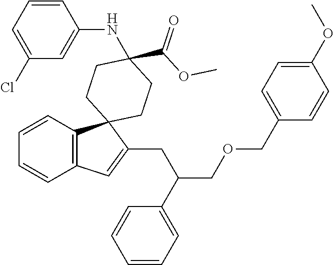 [SMILES: COc1ccc(COCC(CC2=Cc3ccccc3[C@]23CC[C@@](Nc2cccc(Cl)c2)(C(=O)OC)CC3)c2ccccc2)cc1]

Using General procedure 4 and Example 545B as the appropriate bromo compound, an intermediate was obtained, which was used as the appropriate Zn reagent, and treated as described in General procedure 27a, using Preparation 6c as the appropriate 2-bromo-indene derivative to obtain Example 545C as a racemate. ¹H NMR (500 MHz, DMSO-d₆) δ ppm: 7.60 (d, 1H), 7.29 (dm, 2H), 7.24 (tm, 2H), 7.16 (d, 1H), 7.16 (m, 2H), 7.15 (dm, 2H), 7.11 (t, 1H), 7.07 (m, 1H), 6.83 (dm, 2H), 6.68 (t, 1H), 6.62 (dd, 1H), 6.50 (dd, 1H), 6.45 (s, 1H), 6.27 (s, 1H), 4.39/4.36 (d+d, 2H), 3.71 (s, 3H), 3.69 (s, 3H), 3.56 (d, 2H), 3.36 (m, 1H), 2.71/2.55 (dd+dd, 2H), 2.39/2.35/2.22/2.16 (t+d/t+d, 4H), 2.15/2.12/0.88/0.66 (t+d/t+d, 4H). HRMS calculated for C₃₉H₄₀ClNO₄: 621.2646; found 622.2711 (M+H).

### Example 545D methyl (1r,4R)-4-(3-chloroanilino)-2′-[(2R)-3-hydroxy-2-phenylpropyl]spiro[cyclohexane-1,1′-indene]-4-carboxylate

and

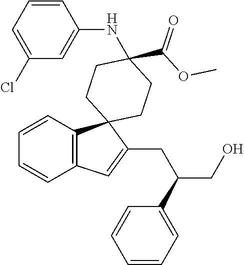 [SMILES: COC(=O)[C@]1(Nc2cccc(Cl)c2)CC[C@]2(CC1)C(C[C@@H](CO)c1ccccc1)=Cc1ccccc12]

### Example 546D methyl (1r,4S)-4-(3-chloroanilino)-2′-[(2S)-3-hydroxy-2-phenylpropyl]spiro[cyclohexane-1,1′-indene]-4-carboxylate

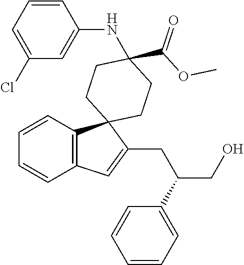 [SMILES: COC(=O)[C@]1(Nc2cccc(Cl)c2)CC[C@]2(CC1)C(C[C@H](CO)c1ccccc1)=Cc1ccccc12]

Using General procedure 28b and Example 545C as the appropriate PMB derivative, a racemate was obtained. The enantiomers were separated by chiral chromatography. Column: AS-V, 100×500 mm, 20 μm, Eluents: 15:85 EtOH/heptane. The enantiomer eluting earlier was collected as Example 545D. ¹H NMR (500 MHz, DMSO-d₆) δ ppm: 7.60 (dm, 1H), 7.25 (dm, 2H), 7.23 (tm, 2H), 7.17 (tm, 1H), 7.15 (m, 2H), 7.12 (t, 1H), 7.06 (dm, 1H), 6.67 (t, 1H), 6.62 (dm, 1H), 6.50 (dm, 1H), 6.45 (s, 1H), 6.27 (s, 1H), 4.75 (t, 1H), 3.69 (s, 3H), 3.58/3.55 (m, 2H), 3.17 (m, 1H), 2.75/2.52 (m, 2H), 2.40-2.16 (m, 4H), 2.15-0.65 (m, 4H). HRMS calculated for C₃₁H₃₂ClNO₃: 501.2071; found 502.2138 (M+H).

The enantiomer eluting later was collected as Example 546D. ¹H NMR (500 MHz, DMSO-d₆) δ ppm: 7.60 (dm, 1H), 7.25 (dm, 2H), 7.23 (tm, 2H), 7.17 (tm, 1H), 7.15 (m, 2H), 7.12 (t, 1H), 7.06 (dm, 1H), 6.67 (t, 1H), 6.62 (dm, 1H), 6.50 (dm, 1H), 6.45 (s, 1H), 6.27 (s, 1H), 4.75 (t, 1H), 3.69 (s, 3H), 3.58/3.55 (m, 2H), 3.17 (m, 1H), 2.75/2.52 (m, 2H), 2.40-2.16 (m, 4H), 2.15-0.65 (m, 4H). HRMS calculated for C₃₁H₃₂ClNO₃: 501.2071; found 502.2147 (M+H).

### Example 548E methyl (1r,2′S,4S)-4-(3-chloroanilino)-2′-[(2R)-3-hydroxy-2-phenylpropyl]-2′,3′-dihydrospiro[cyclohexane-1,1′-indene]-4-carboxylate

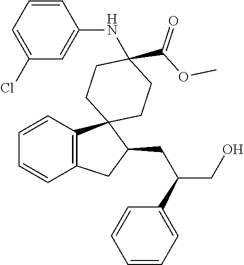 [SMILES: COC(=O)[C@]1(Nc2cccc(Cl)c2)CC[C@@]2(CC1)c1ccccc1C[C@@H]2C[C@@H](CO)c1ccccc1]

and

### Example 545E methyl (1r,2′R,4R)-4-(3-chloroanilino)-2′-[(2R)-3-hydroxy-2-phenylpropyl]-2′,3′-dihydrospiro[cyclohexane-1,1′-indene]-4-carboxylate

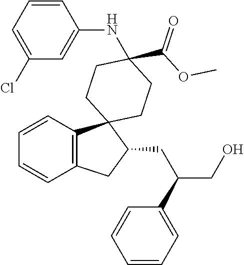 [SMILES: COC(=O)[C@]1(Nc2cccc(Cl)c2)CC[C@@]2(CC1)c1ccccc1C[C@H]2C[C@@H](CO)c1ccccc1]

Using General procedure 19 and Example 545D as the appropriate indene, a mixture of diastereoisomers was obtained. They were separated by chiral chromatography. Column: AD, 50×500 mm, 20 μm, Eluents: 25:75 iPrOH/heptane. The diastereoisomer eluting earlier was collected as Example 548E. ¹H NMR (500 MHz, DMSO-d₆) δ ppm: 7.38 (d, 1H), 7.27 (tm, 2H), 7.23 (dm, 2H), 7.18 (tm, 1H), 7.15-7.04 (m, 3H), 7.07 (t, 1H), 6.63 (t, 1H), 6.58 (dm, 1H), 6.47 (dm, 1H), 6.32 (s, 1H), 4.58 (t, 1H), 3.65 (s, 3H), 3.65-3.53 (m, 2H), 2.73 (m, 1H), 2.64/2.35 (dd+dd, 2H), 2.5-1.18 (m, 8H), 2.08 (m, 1H), 2/1.35 (m+m, 2H). HRMS calculated for C₃₁H₃₄ClNO₃: 503.2227; found 504.2299 (M+H).

The diastereoisomer eluting later was collected as Example 545E. ¹H NMR (500 MHz, DMSO-d₆) δ ppm: 7.34 (m, 1H), 7.27 (tm, 2H), 7.23 (dm, 2H), 7.2-7.08 (m, 3H), 7.16 (tm, 1H), 7.08 (t, 1H), 6.62 (t, 1H), 6.58 (dm, 1H), 6.46 (dm, 1H), 6.29 (s, 1H), 4.66 (t, 1H), 3.63 (s, 3H), 3.5 (m, 2H), 2.85/2.57 (dd+dd, 2H), 2.77 (m, 1H), 2.4-1.12 (m, 8H), 2.35/1.73 (m+m, 2H), 1.55 (m, 1H). HRMS calculated for C₃₁H₃₄ClNO₃: 503.2227; found 504.2301 (M+H).

### Example 546E methyl (1r,2′R,4R)-4-(3-chloroanilino)-2′-[(2S)-3-hydroxy-2-phenylpropyl]-2′,3′-dihydrospiro[cyclohexane-1,1′-indene]-4-carboxylate

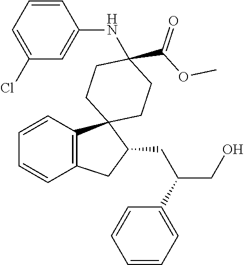 [SMILES: COC(=O)[C@]1(Nc2cccc(Cl)c2)CC[C@@]2(CC1)c1ccccc1C[C@H]2C[C@H](CO)c1ccccc1]

and

### Example 547E methyl (1r,2′S,4S)-4-(3-chloroanilino)-2′-[(2S)-3-hydroxy-2-phenylpropyl]-2′,3′-dihydrospiro[cyclohexane-1,1′-indene]-4-carboxylate

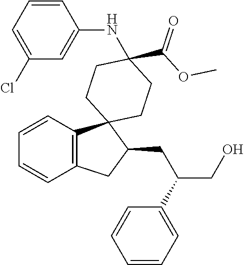 [SMILES: COC(=O)[C@]1(Nc2cccc(Cl)c2)CC[C@@]2(CC1)c1ccccc1C[C@@H]2C[C@H](CO)c1ccccc1]

Using General procedure 19 and Example 546D as the appropriate indene, a mixture of diastereoisomers was obtained. They were separated by chiral chromatography. Column: AD, 50×500 mm, 20 μm, Eluents: 25:75 iPrOH/heptane. The diastereoisomer eluting earlier was collected as Example 546E. ¹H NMR (500 MHz, DMSO-d₆) δ ppm: 7.38 (d, 1H), 7.27 (tm, 2H), 7.23 (dm, 2H), 7.18 (tm, 1H), 7.15-7.04 (m, 3H), 7.07 (t, 1H), 6.63 (t, 1H), 6.58 (dm, 1H), 6.47 (dm, 1H), 6.32 (s, 1H), 4.58 (t, 1H), 3.65 (s, 3H), 3.65-3.53 (m, 2H), 2.73 (m, 1H), 2.64/2.35 (dd+dd, 2H), 2.50-1.18 (m, 8H), 2.08 (m, 1H), 2.00/1.35 (m+m, 2H). HRMS calculated for C31 H34Cl N O3: 503.2227; found 504.2297 (M+H).

The diastereoisomer eluting later was collected as Example 547E. ¹H NMR (500 MHz, DMSO-d₆) δ ppm: 7.34 (m, 1H), 7.27 (tm, 2H), 7.23 (dm, 2H), 7.20-7.08 (m, 3H), 7.16 (tm, 1H), 7.08 (t, 1H), 6.62 (t, 1H), 6.58 (dm, 1H), 6.46 (dm, 1H), 6.29 (s, 1H), 4.66 (t, 1H), 3.63 (s, 3H), 3.50 (m, 2H), 2.85/2.57 (dd+dd, 2H), 2.77 (m, 1H), 2.40-1.12 (m, 8H), 2.35/1.73 (m+m, 2H), 1.55 (m, 1H). HRMS calculated for C₃₁H₃₄ClNO₃: 503.2227; found 504.2297 (M+H).

### Example 545 (1r,2′S,4S)-4-(3-chloroanilino)-2′-{(2R)-2-phenyl-3-[(thieno[3,2-b]pyridin-7-yl)oxy]propyl}-2′,3′-dihydrospiro[cyclohexane-1,1′-indene]-4-carboxylic acid

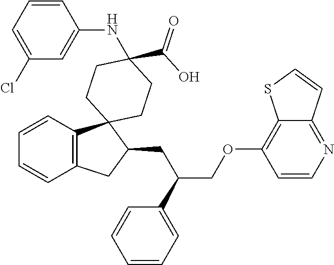 [SMILES: O=C(O)[C@]1(Nc2cccc(Cl)c2)CC[C@@]2(CC1)c1ccccc1C[C@@H]2C[C@@H](COc1ccnc2ccsc12)c1ccccc1]

Using General procedure 32 and Example 548E as the appropriate indane and thieno[3,2-b]pyridin-7-ol as the appropriate alcohol, Example 545 was obtained. ¹H NMR (500 MHz, DMSO-d₆) δ ppm: 12.68 (br s, 1H), 8.48 (d, 1H), 7.85 (d, 1H), 7.45 (d, 1H), 7.44-7.20 (m, 5H), 7.43-7.07 (m, 4H), 7.04 (t, 1H), 6.99 (d, 1H), 6.63 (t, 1H), 6.55 (dm, 1H), 6.53 (dm, 1H), 6.14 (br s, 1H), 4.49/4.45 (m+m, 2H), 3.27 (m, 1H), 2.79/2.46 (dd+dd, 2H), 2.50-1.20 (m, 8H), 2.23/1.61 (m+m, 2H), 2.18 (m, 1H). HRMS calculated for C₃₇H₃₅N₂O₃SCl: 622.2057; found: 623.2132 (M+H).

### Example 546 (1r,2′R,4R)-4-(3-chloroanilino)-2′-{(2S)-2-phenyl-3-[(thieno[3,2-b]pyridin-7-yl)oxy]propyl}-2′,3′-dihydrospiro[cyclohexane-1,1′-indene]-4-carboxylic acid

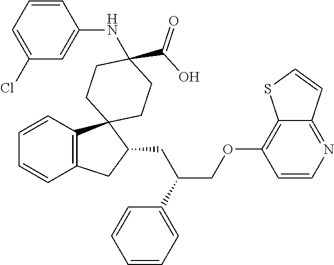 [SMILES: O=C(O)[C@]1(Nc2cccc(Cl)c2)CC[C@@]2(CC1)c1ccccc1C[C@H]2C[C@H](COc1ccnc2ccsc12)c1ccccc1]

Using General procedure 32 and Example 546E as the appropriate indane and thieno[3,2-b]pyridin-7-ol as the appropriate alcohol, Example 546 was obtained. ¹H NMR (500 MHz, DMSO-d₆) δ ppm: 12.68 (br s, 1H), 8.48 (d, 1H), 7.85 (d, 1H), 7.45 (d, 1H), 7.44-7.08 (m, 9H), 7.04 (t, 1H), 6.99 (d, 1H), 6.63 (t, 1H), 6.55 (dm, 1H), 6.52 (dm, 1H), 6.14 (br s, 1H), 4.49/4.45 (dd+dd, 2H), 3.27 (m, 1H), 2.79/2.46 (dd+dd, 2H), 2.48-1.22 (m, 8H), 2.23/1.6 (m+m, 2H), 2.18 (m, 1H). HRMS calculated for C₃₇H₃₅N₂O₃SCl: 622.2057; found: 623.2162 (M+H).

### Example 547 (1r,2′S,4S)-4-(3-chloroanilino)-2′-{(2S)-2-phenyl-3-[(thieno[3,2-b]pyridin-7-yl)oxy]propyl}-2′,3′-dihydrospiro[cyclohexane-1,1′-indene]-4-carboxylic acid

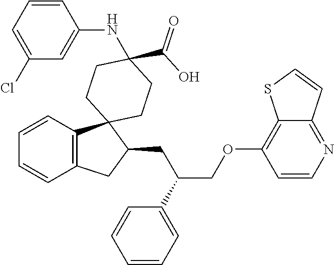 [SMILES: O=C(O)[C@]1(Nc2cccc(Cl)c2)CC[C@@]2(CC1)c1ccccc1C[C@@H]2C[C@H](COc1ccnc2ccsc12)c1ccccc1]

Using General procedure 32 and Example 547E as the appropriate indane and thieno[3,2-b]pyridin-7-ol as the appropriate alcohol, Example 547 was obtained. ¹H NMR (500 MHz, DMSO-d₆) δ ppm: 12.66 (br s, 1H), 8.49 (d, 1H), 7.99 (d, 1H), 7.48 (d, 1H), 7.44-6.97 (m, 9H), 7.04 (t, 1H), 7.01 (d, 1H), 6.63 (t, 1H), 6.54 (dm, 1H), 6.54 (dm, 1H), 6.19 (br s, 1H), 4.47/4.40 (dd+dd, 2H), 3.28 (m, 1H), 2.95/2.65 (dd+dd, 2H), 2.43-1.15 (m, 8H), 2.04/1.81 (m+m, 2H), 1.63 (m, 1H). HRMS calculated for C₃₇H₃₅N₂O₃SCl: 622.2057; found: 623.2124 (M+H).

### Example 548 (1r,2′S,4S)-4-(3-chloroanilino)-2′-{(2R)-2-phenyl-3-[(pyridin-3-yl)oxy]propyl}-2′,3′-dihydrospiro[cyclohexane-1,1′-indene]-4-carboxylic acid

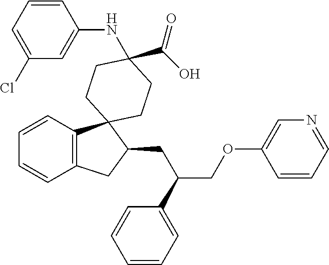 [SMILES: O=C(O)[C@]1(Nc2cccc(Cl)c2)CC[C@@]2(CC1)c1ccccc1C[C@@H]2C[C@@H](COc1cccnc1)c1ccccc1]

Using General procedure 32 and Example 548E as the appropriate indane and pyridin-3-ol as the appropriate alcohol, Example 548 was obtained. HRMS calculated for C₃₅H₃₅N₂O₃Cl: 566.2336; found: 567.2408 (M+H).

### Example 549 (1r,2′R,4R)-4-(3-chloroanilino)-2′-{(2R)-2-phenyl-3-[(pyridin-3-yl)oxy]propyl}-2′,3′-dihydrospiro[cyclohexane-1,1′-indene]-4-carboxylic acid

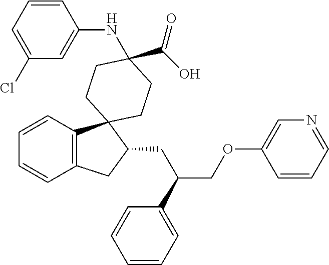 [SMILES: O=C(O)[C@]1(Nc2cccc(Cl)c2)CC[C@@]2(CC1)c1ccccc1C[C@H]2C[C@@H](COc1cccnc1)c1ccccc1]

Using General procedure 32 and Example 545E as the appropriate indane and pyridin-3-ol as the appropriate alcohol, Example 549 was obtained. ¹H NMR (500 MHz, DMSO-d₆) δ ppm: 12.69 (br s, 1H), 8.23 (d, 1H), 8.13 (dd, 1H), 7.40-7.07 (m, 11H), 7.05 (t, 1H), 6.63 (t, 1H), 6.55 (dm, 1H), 6.55 (dm, 1H), 6.19 (br s, 1H), 4.22/4.20 (dd+dd, 2H), 3.18 (m, 1H), 2.90/2.62 (dd+dd, 2H), 2.42-1.12 (m, 8H), 1.93/1.78 (m+m, 2H), 1.58 (m, 1H). HRMS calculated for C₃₅H₃₅N₂O₃Cl: 566.2336; found: 567.2402 (M+H).

### Example 550 (1r,2′R,4R)-4-(3-chloroanilino)-2′-{(2S)-2-phenyl-3-[(pyridin-3-yl)oxy]propyl}-2′,3′-dihydrospiro[cyclohexane-1,1′-indene]-4-carboxylic acid

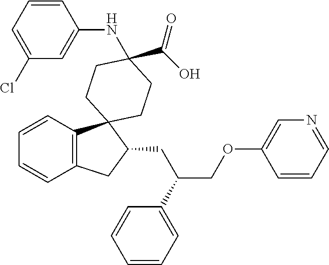 [SMILES: O=C(O)[C@]1(Nc2cccc(Cl)c2)CC[C@@]2(CC1)c1ccccc1C[C@H]2C[C@H](COc1cccnc1)c1ccccc1]

Using General procedure 32 and Example 546E as the appropriate indane and pyridin-3-ol as the appropriate alcohol, Example 550 was obtained. HRMS calculated for C₃₅H₃₅N₂O₃Cl: 566.2336; found: 567.2406 (M+H).

### Example 551 (1r,2′S,4S)-4-(3-chloroanilino)-2′-{(2S)-2-phenyl-3-[(pyridin-3-yl)oxy]propyl}-2′,3′-dihydrospiro[cyclohexane-1,1′-indene]-4-carboxylic acid

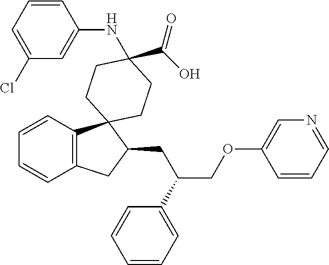 [SMILES: O=C(O)[C@]1(Nc2cccc(Cl)c2)CC[C@@]2(CC1)c1ccccc1C[C@@H]2C[C@H](COc1cccnc1)c1ccccc1]

Using General procedure 32 and Example 547E as the appropriate indane and pyridin-3-ol as the appropriate alcohol, Example 551 was obtained. HRMS calculated for C₃₅H₃₅N₂O₃Cl: 566.2336; found: 567.2399 (M+H).

### Example 552 (1r,2′S,4S)-4-(3-chloroanilino)-2′-{(2R)-2-phenyl-3-[(pyridin-4-yl)oxy]propyl}-2′,3′-dihydrospiro[cyclohexane-1,1′-indene]-4-carboxylic acid

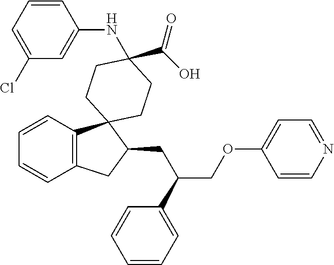 [SMILES: O=C(O)[C@]1(Nc2cccc(Cl)c2)CC[C@@]2(CC1)c1ccccc1C[C@@H]2C[C@@H](COc1ccncc1)c1ccccc1]

Using General procedure 32 and Example 548E as the appropriate indane and pyridin-4-ol as the appropriate alcohol, Example 552 was obtained. ¹H NMR (500 MHz, DMSO-d₆) δ ppm: 12.69 (br s, 1H), 8.32 (m, 2H), 7.43-7.04 (m, 9H), 7.05 (t, 1H), 6.94 (m, 2H), 6.65 (t, 1H), 6.56 (dm, 1H), 6.54 (dm, 1H), 6.24 (br s, 1H), 4.26 (d, 2H), 3.16 (m, 1H), 2.63/2.41 (dd+dd, 2H), 2.48-1.22 (m, 8H), 2.16/1.52 (m+m, 2H), 2.09 (m, 1H). HRMS calculated for C₃₅H₃₅N₂O₃Cl: 566.2336; found: 567.2406 (M+H).

### Example 553 (1r,2′R,4R)-4-(3-chloroanilino)-2′-{(2R)-2-phenyl-3-[(pyridin-4-yl)oxy]propyl}-2′,3′-dihydrospiro[cyclohexane-1,1′-indene]-4-carboxylic acid

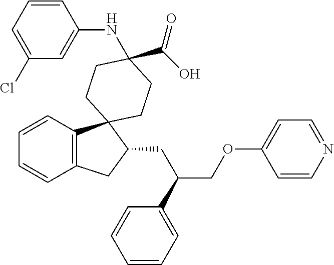 [SMILES: O=C(O)[C@]1(Nc2cccc(Cl)c2)CC[C@@]2(CC1)c1ccccc1C[C@H]2C[C@@H](COc1ccncc1)c1ccccc1]

Using General procedure 32 and Example 545E as the appropriate indane and pyridin-4-ol as the appropriate alcohol, Example 553 was obtained. ¹H NMR (500 MHz, DMSO-d₆) δ ppm: 12.64 (br s, 1H), 8.33 (m, 2H), 7.40-7.09 (m, 9H), 7.06 (t, 1H), 6.92 (m, 2H), 6.63 (t, 1H), 6.56 (dm, 1H), 6.54 (dm, 1H), 6.19 (br s, 1H), 4.24/4.21 (dd+dd, 2H), 3.18 (m, 1H), 2.90/2.62 (dd+dd, 2H), 2.41-1.13 (m, 8H), 1.93/1.77 (m+m, 2H), 1.57 (m, 1H). HRMS calculated for C₃₅H₃₅N₂O₃Cl: 566.2336; found: 567.2412 (M+H).

### Example 554 (1r,2′R,4R)-4-(3-chloroanilino)-2′-{(2S)-2-phenyl-3-[(pyridin-4-yl)oxy]propyl}-2′,3′-dihydrospiro[cyclohexane-1,1′-indene]-4-carboxylic acid

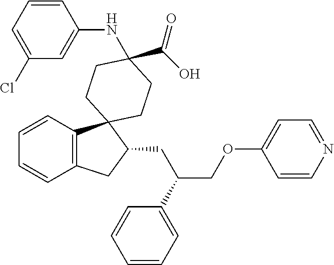 [SMILES: O=C(O)[C@]1(Nc2cccc(Cl)c2)CC[C@@]2(CC1)c1ccccc1C[C@H]2C[C@H](COc1ccncc1)c1ccccc1]

Using General procedure 32 and Example 546E as the appropriate indane and pyridin-4-ol as the appropriate alcohol, Example 554 was obtained. HRMS calculated for C₃₅H₃₅N₂O₃Cl: 566.2336; found: 567.2410 (M+H).

### Example 555 (1r,2′S,4S)-4-(3-chloroanilino)-2′-{(2S)-2-phenyl-3-[(pyridin-4-yl)oxy]propyl}-2′,3′-dihydrospiro[cyclohexane-1,1′-indene]-4-carboxylic acid

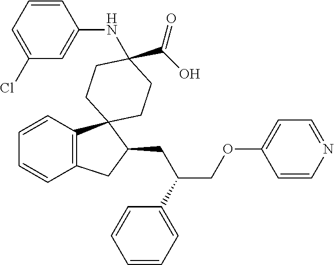 [SMILES: O=C(O)[C@]1(Nc2cccc(Cl)c2)CC[C@@]2(CC1)c1ccccc1C[C@@H]2C[C@H](COc1ccncc1)c1ccccc1]

Using General procedure 32 and Example 547E as the appropriate indane and pyridin-4-ol as the appropriate alcohol, Example 555 was obtained. HRMS calculated for C₃₅H₃₅N₂O₃Cl: 566.2336; found: 567.2403 (M+H).

### Example 556 (1r,2′S,4S)-4-(3-chloroanilino)-2′-[(2R)-3-phenoxy-2-phenylpropyl]-2′,3′-dihydrospiro[cyclohexane-1,1′-indene]-4-carboxylic acid

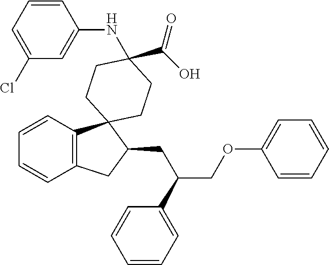 [SMILES: O=C(O)[C@]1(Nc2cccc(Cl)c2)CC[C@@]2(CC1)c1ccccc1C[C@@H]2C[C@@H](COc1ccccc1)c1ccccc1]

Using General procedure 32 and Example 548E as the appropriate indane and phenol as the appropriate alcohol, Example 556 was obtained. ¹H NMR (500 MHz, DMSO-d₆) δ ppm: 12.68 (br s, 1H), 7.43-7.03 (m, 9H), 7.23 (m, 2H), 7.05 (t, 1H), 6.91 (m, 2H), 6.89 (m, 1H), 6.65 (t, 1H), 6.55 (dm, 1H), 6.55 (dm, 1H), 6.23 (br s, 1H), 4.16 (d, 2H), 3.12 (m, 1H), 2.64/2.41 (dd+dd, 2H), 2.48-1.21 (m, 8H), 2.18/1.50 (m+m, 2H), 2.10 (m, 1H). HRMS calculated for C₃₆H₃₆NO₃Cl: 565.2383; found: 566.2446 (M+H).

### Example 557 (1r,2′R,4R)-4-(3-chloroanilino)-2′-[(2S)-3-phenoxy-2-phenylpropyl]-2′,3′-dihydrospiro[cyclohexane-1,1′-indene]-4-carboxylic acid

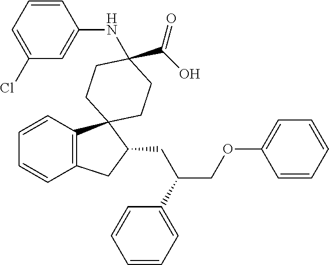 [SMILES: O=C(O)[C@]1(Nc2cccc(Cl)c2)CC[C@@]2(CC1)c1ccccc1C[C@H]2C[C@H](COc1ccccc1)c1ccccc1]

Using General procedure 32 and Example 546E as the appropriate indane and phenol as the appropriate alcohol Example 557 was obtained. HRMS calculated for C₃₆H₃₆NO₃Cl: 565.2383; found: 566.2451 (M+H).

### Example 558

### Example 558A tert-butyl 4-{[(1r,4r)-4-(3-chloroanilino)-4-(methoxycarbonyl)spiro[cyclohexane-1,1′-inden]-2′-yl]methyl}piperidine-1-carboxylate

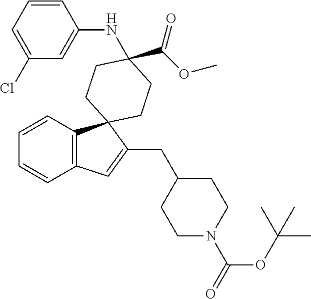 [SMILES: COC(=O)[C@]1(Nc2cccc(Cl)c2)CC[C@]2(CC1)C(CC1CCN(C(=O)OC(C)(C)C)CC1)=Cc1ccccc12]

To an oven-dried vial was added Preparation 6c (113 mg, 0.25 mmol, 1 eq), tert-butyl 4-(bromomethyl)piperidine-1-carboxylate (106 mg, 0.38 mmol, 1.5 eq), (4,4′-di-t-butyl-2,2′-bipyridine)bis[3,5-difluoro-2-[5-trifluoromethyl-2-pyridinyl-kN)phenyl-kC]iridium(III) hexafluorophosphate (2.8 mg, 2.53 μmol, 0.01 eq) and Na₂CO₃ (54 mg, 0.51 mmol, 2 eq). The mixture was evacuated and flushed with N₂ (×3) before the addition of DME (2 mL), followed by tris(trimethylsilyl)silane (0.08 mL, 0.25 mmol, 1 eq). To a separate oven-dried vial was added NiCl₂×glyme (2.8 mg, 0.01 mmol, 0.05 eq) and 4,4′-di-tert-butyl-2,2′-dipyridyl (3.4 mg, 0.01 mmol, 0.05 eq) and the vessel was evacuated and flushed with N₂ (×3). DME (1 mL) was added and the suspension was stirred for 5 min at rt. The nickel complex solution (0.1 mL) was added to the main reaction mixture, sparged with N₂ and irradiated with a 34 W blue LED lamp (450 nm), with compressed air cooling for 6.25 h. Purification by automated flash chromatography (CombiFlash Rf, 12 g RediSep™ silica cartridge) eluting with a gradient of 0-50% EtOAc in heptane afforded Example 558A as a white solid (53 mg, 0.09 mmol, 37%). ¹H NMR (400 MHz, CDCl₃) δ ppm: 7.72 (d, J=7.5 Hz, 1H), 7.31-7.23 (m, 2H), 7.17-7.06 (m, 2H), 6.77-6.72 (m, 1H), 6.63 (t, J=2.1 Hz, 1H), 6.52-6.47 (m, 1H), 6.45-6.42 (m, 1H), 4.25-3.96 (br m, 3H), 3.77 (s, 3H), 2.81-2.52 (m, 4H), 2.31-2.20 (m, 2H), 2.19-2.11 (m, 2H), 2.04 (td, J=13.8, 4.0 Hz, 2H), 1.90-1.73 (m, 3H), 1.46 (s, 9H), 1.22-1.03 (m, 4H).

### Example 558 (1r,4r)-2′-{[1-(tert-butoxycarbonyl)piperidin-4-yl]methyl}-4-(3-chloroanilino)spiro[cyclohexane-1,1′-indene]-4-carboxylic acid

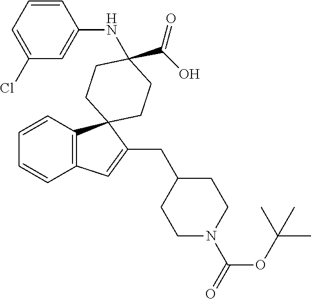 [SMILES: CC(C)(C)OC(=O)N1CCC(CC2=Cc3ccccc3[C@]23CC[C@@](Nc2cccc(Cl)c2)(C(=O)O)CC3)CC1]

To a solution of Example 558A (51 mg, 0.09 mmol, 1 eq) in MeOH (2 mL) was added LiOH×H₂O (27 mg, 0.64 mmol, 7 eq) and the mixture was heated at 120° C. for 45 min under microwave irradiation. After cooling to rt, sat. aq. NH₄Cl solution was added, and the suspension was stirred for 5 min. After cooling to 0° C. for 5 min, the solids were collected by filtration, washed with water and MeCN and dried under vacuum to afford Example 558 as a white solid (33.5 mg, 0.06 mmol, 67%). ¹H NMR (400 MHz, DMSO-d₆) δ ppm: 7.73 (d, J=7.6 Hz, 1H), 7.30-7.24 (m, 1H), 7.24-7.17 (m, 1H), 7.12-7.05 (m, 1H), 7.01 (t, J=8.0 Hz, 1H), 6.65 (t, J=2.1 Hz, 1H), 6.62-6.56 (m, 1H), 6.50-6.43 (m, 2H), 6.11 (br s, 1H), 4.03-3.84 (br m, 2H), 2.85-2.59 (br m, 2H), 2.45-2.31 (m, 2H), 2.22-1.98 (m, 6H), 1.92-1.66 (m, 3H), 1.39 (s, 9H), 1.11-0.96 (m, 2H), 0.85-0.74 (m, 2H).

### Example 559

### Example 559A 2-benzyl-3-[(4-methoxyphenyl)methoxy]propan-1-ol

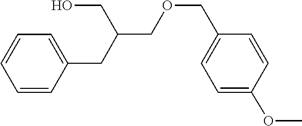 [SMILES: COc1ccc(COCC(CO)Cc2ccccc2)cc1]

Using General procedure 2 and 2-benzylpropane-1,3-diol as the appropriate diol, Example 559A was obtained as a racemate. MS (EI, 70 eV) m/z (% relative intensity, [ion]): 91 (18), 121 (100), 137 (33), 286 (2, [M+]).

### Example 559B 1-[(2-benzyl-3-bromo-propoxy)methyl]-4-methoxy-benzene

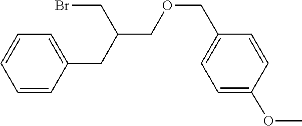 [SMILES: COc1ccc(COCC(CBr)Cc2ccccc2)cc1]

Using General procedure 3 and Example 559A as the appropriate alcohol, Example 559B was obtained as a racemate. ¹H NMR (400 MHz, DMSO-d₆) δ ppm: 7.32-7.15 (m, 7H), 6.93-6.87 (m, 2H), 4.38 (q, 2H), 3.74 (s, 3H), 3.58/3.42 (dd, 2H), 3.30-3.30 (m, 2H), 2.63 (h, 2H), 2.26-2.14 (m, 1H). MS (EI, 70 eV) m/z (% relative intensity, [ion]): 91 (15), 121 (100), 137 (3), 348 (1, [M+]), 350 (1, [M+]).

### Example 559C methyl (1r,4r)-2′-{2-benzyl-3-[(4-methoxyphenyl)methoxy]propyl}-4-(3-chloroanilino)spiro[cyclohexane-1,1′-indene]-4-carboxylate

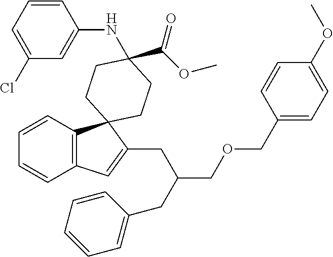 [SMILES: COc1ccc(COCC(Cc2ccccc2)CC2=Cc3ccccc3[C@]23CC[C@@](Nc2cccc(Cl)c2)(C(=O)OC)CC3)cc1]

Using General procedure 4 and Example 559B as the appropriate bromo compound, an intermediate was obtained, which was used as the appropriate Zn reagent, and treated as described in General procedure 27a, using Preparation 6c as the appropriate 2-bromo-indene derivative to obtain Example 559C as a racemate. ¹H NMR (500 MHz, DMSO-d₆) δ ppm: 7.66-7.07 (m, 4H), 7.29-7.13 (m, 5H), 7.17 (m, 2H), 7.11 (t, 1H), 6.81 (m, 2H), 6.65 (t, 1H), 6.62 (dm, 1H), 6.51 (t, 1H), 6.48 (dm, 1H), 6.43 (s, 1H), 4.31/4.29 (d+d, 2H), 3.72 (s, 3H), 3.69 (s, 3H), 3.27/3.25 (dd+dd, 2H), 2.74-2.63 (m, 2H), 2.45-0.75 (m, 8H), 2.33 (m, 1H), 2.33/2.15 (m+m, 2H). HRMS calculated for C₄₀H₄₂ClNO₄: 635.2802; found: 636.2863 (M+H).

### Example 559D methyl (1r,4r)-2′-(2-benzyl-3-hydroxypropyl)-4-(3-chloroanilino)spiro[cyclohexane-1,1′-indene]-4-carboxylate, enantiomer 1

and

### Example 559E methyl (1r,4r)-2′-(2-benzyl-3-hydroxypropyl)-4-(3-chloroanilino)spiro[cyclohexane-1,1′-indene]-4-carboxylate, enantiomer 2

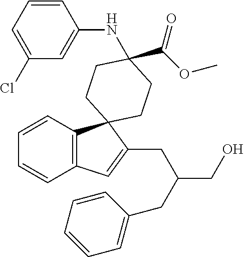 [SMILES: COC(=O)[C@]1(Nc2cccc(Cl)c2)CC[C@]2(CC1)C(CC(CO)Cc1ccccc1)=Cc1ccccc12]

Using General procedure 28b and Example 559C as the appropriate PMB derivative, a racemate was obtained. The enantiomers were separated by chiral chromatography. Column: AD, 50×500 mm, 20 μm, eluents: 50:50 iPrOH/heptane. The enantiomer eluting earlier was collected as Example 559D. ¹H NMR (500 MHz, DMSO-d₆) δ ppm: 7.64 (d, 1H), 7.30 (d, 1H), 7.26 (dm, 2H), 7.22 (t, 1H), 7.21 (tm, 2H), 7.15 (tm, 1H), 7.11 (t, 1H), 7.10 (t, 1H), 6.64 (t, 1H), 6.61 (dd, 1H), 6.55 (s, 1H), 6.47 (dd, 1H), 6.42 (s, 1H), 4.55 (t, 1H), 3.69 (s, 3H), 3.31 (m, 2H), 2.67/2.62 (m+m, 2H), 2.39/2.35/2.10/0.87 (m, 8H), 2.20/2.08 (m+m, 2H), 2.17 (m, 1H). HRMS calculated for C₃₂H₃₄ClNO₃: 515.2227; found: 516.2293 (M+H).

The enantiomer eluting later was collected as Example 559E. HRMS calculated for C₃₂H₃₄ClNO₃: 515.2227; found: 516.2290 (M+H).

### Example 559 (1r,4r)-2′-{2-benzyl-3-[(thieno[3,2-b]pyridin-7-yl)oxy]propyl}-4-(3-chloroanilino)spiro[cyclohexane-1,1′-indene]-4-carboxylic acid, enantiomer 1

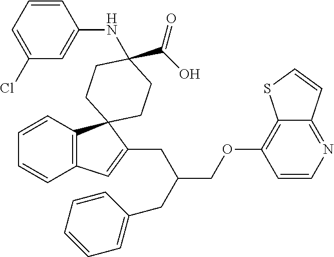 [SMILES: O=C(O)[C@]1(Nc2cccc(Cl)c2)CC[C@]2(CC1)C(CC(COc1ccnc3ccsc13)Cc1ccccc1)=Cc1ccccc12]

Using General procedure 32 and Example 559D as the appropriate indene and thieno[3,2-b]pyridin-7-ol as the appropriate alcohol, Example 559 was obtained. ¹H NMR (500 MHz, DMSO-d₆) δ ppm: 12.81 (br s, 1H), 8.43 (d, 1H), 7.87 (d, 1H), 7.68-7.07 (m, 4H), 7.47 (d, 1H), 7.31-7.15 (m, 5H), 7.10 (t, 1H), 6.83 (d, 1H), 6.66 (br s, 1H), 6.65 (t, 1H), 6.60 (dm, 1H), 6.56 (dm, 1H), 6.34 (br s, 1H), 4.14/4.10 (dd+dd, 2H), 2.89/2.85 (dd+dd, 2H), 2.70 (m, 1H), 2.52/2.41 (dd+dd, 2H), 2.44-0.66 (m, 8H). HRMS calculated for C₃₈H₃₅N₂O₃SCl: 634.2057; found: 635.2132 (M+H).

### Example 560 and Example 561 and Example 562 and Example 563

### Example 560A methyl (1r,4r)-2′-(2-benzyl-3-hydroxypropyl)-4-(3-chloroanilino)-2′,3′-dihydrospiro[cyclohexane-1,1′-indene]-4-carboxylate, diastereoisomer 1 enantiomer 1

and

### Example 561A methyl (1r,4r)-2′-(2-benzyl-3-hydroxypropyl)-4-(3-chloroanilino)-2′,3′-dihydrospiro[cyclohexane-1,1′-indene]-4-carboxylate, diastereoisomer 2 enantiomer 1

and

### Example 562A methyl (1r,4r)-2′-(2-benzyl-3-hydroxypropyl)-4-(3-chloroanilino)-2′,3′-dihydrospiro[cyclohexane-1,1′-indene]-4-carboxylate, diastereoisomer 2 enantiomer 2

and

### Example 563A methyl (1r,4r)-2′-(2-benzyl-3-hydroxypropyl)-4-(3-chloroanilino)-2′,3′-dihydrospiro[cyclohexane-1,1′-indene]-4-carboxylate, diastereoisomer 1 enantiomer 2

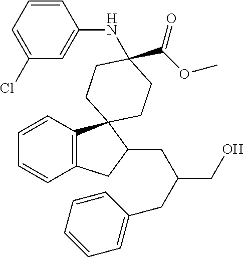

Using General procedure 19 and Example 559D as the appropriate indene, a mixture of diastereoisomers was obtained. The diastereoisomers were separated via flash chromatography using DCM and MeOH as eluents. The diastereoisomer eluting earlier was collected as Example 560A. ¹H NMR (500 MHz, DMSO-d₆) δ ppm: 7.38-7.09 (m, 9H), 7.08 (t, 1H), 6.62 (t, 1H), 6.58 (dm, 1H), 6.47 (dm, 1H), 6.31 (s, 1H), 4.46 (t, 1H), 3.65 (s/s, 3H), 3.28/3.17 (m+m, 2H), 2.95/2.54 (dd+dd, 2H), 2.66/2.50 (dd+dd, 2H), 2.49-1.26 (m, 10H), 2.09 (m, 1H), 1.75 (m, 1H). HRMS calculated for C₃₂H₃₆ClNO₃: 517.2383; found: 518.2452 (M+H).

The diastereoisomer eluting later was collected as Example 561A. ¹H NMR (500 MHz, DMSO-d₆) δ ppm: 7.36-7.08 (m, 9H), 7.08 (t, 1H), 6.60 (t/t, 1H), 6.58 (dm, 1H), 6.45 (dm, 1H), 6.29 (s/s, 1H), 4.48 (t, 1H), 3.64 (s/s, 3H), 3.41/3.31 (m+m, 2H), 2.89/2.28 (dd+dd, 2H), 2.8/2.40 (dd+dd, 2H), 2.47-1.02 (m, 10H), 2.11 (m, 1H), 1.75 (m, 1H). HRMS calculated for C₃₂H₃₆ClNO₃: 517.2383; found: 518.2452 (M+H).

Using General procedure 19 and Example 559E as the appropriate indene, a mixture of diastereoisomers was obtained. The diastereoisomers were separated by chiral chromatography. Column: IC, 50×500 mm, 20 μm, Eluents: 10:90 nPrOH/heptane. The diastereoisomer eluting earlier was collected as Example 562A. HRMS calculated for C₃₂H₃₆ClNO₃: 517.2383; found: 518.2457 (M+H).

The diastereoisomer eluting later was collected as Example 563A. HRMS calculated for C₃₂H₃₆ClNO₃: 517.2383; found: 518.2453 (M+H).

### Example 560 (1r,4r)-2′-{2-benzyl-3-[(thieno[3,2-b]pyridin-7-yl)oxy]propyl}-4-(3-chloroanilino)-2′,3′-dihydrospiro[cyclohexane-1,1′-indene]-4-carboxylic acid, diastereoisomer 1 enantiomer 1

and

### Example 561 (1r,4r)-2′-{2-benzyl-3-[(thieno[3,2-b]pyridin-7-yl)oxy]propyl}-4-(3-chloroanilino)-2′,3′-dihydrospiro[cyclohexane-1,1′-indene]-4-carboxylic acid, diastereoisomer 2 enantiomer 1

and

### Example 562 (1r,4r)-2′-{2-benzyl-3-[(thieno[3,2-b]pyridin-7-yl)oxy]propyl}-4-(3-chloroanilino)-2′,3′-dihydrospiro[cyclohexane-1,1′-indene]-4-carboxylic acid, diastereoisomer 2 enantiomer 2

and

### Example 563 (1r,4r)-2′-{2-benzyl-3-[(thieno[3,2-b]pyridin-7-yl)oxy]propyl}-4-(3-chloroanilino)-2′,3′-dihydrospiro[cyclohexane-1,1′-indene]-4-carboxylic acid, diastereoisomer 1 enantiomer 2

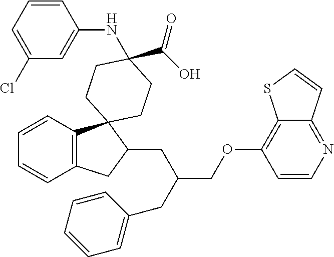

Using General procedure 32 and Example 560A as the appropriate indane and thieno[3,2-b]pyridin-7-ol as the appropriate alcohol, Example 560 was obtained. ¹H NMR (500 MHz, DMSO-d₆) δ ppm: 12.69 (br s, 1H), 8.47 (d, 1H), 8.02 (d, 1H), 7.51 (d, 1H), 7.40-7.10 (m, 4H), 7.27-7.10 (m, 5H), 7.05 (t, 1H), 6.86 (d, 1H), 6.64 (t, 1H), 6.55 (dm, 1H), 6.55 (dm, 1H), 6.24 (br s, 1H), 4.10/4.05 (dd+dd, 2H), 3.04/2.60 (dd+dd, 2H), 2.90/2.72 (dd+dd, 2H), 2.48-1.28 (m, 10H), 2.25 (m, 1H), 2.14 (m, 1H). HRMS calculated for C₃₈H₃₇N₂O₃SCl: 636.2213; found: 637.2293 (M+H).

Using General procedure 32 and Example 561A as the appropriate indane and thieno[3,2-b]pyridin-7-ol as the appropriate alcohol, Example 561 was obtained. ¹H NMR (500 MHz, DMSO-d₆) δ ppm: 12.67 (br s, 1H), 8.47 (d, 1H), 7.90 (d, 1H), 7.48 (d, 1H), 7.40-7.08 (m, 9H), 7.06 (t, 1H), 6.90 (d, 1H), 6.59 (t, 1H), 6.56 (dm, 1H), 6.49 (dm, 1H), 6.09 (br s, 1H), 4.19/4.15 (dd+dd, 2H), 3.01/2.43 (dd+dd, 2H), 2.87/2.73 (dd+dd, 2H), 2.46-1.16 (m, 10H), 2.29 (m, 1H), 2.16 (m, 1H). HRMS calculated for C₃₈H₃₇N₂O₃SCl: 636.2213; found: 637.2276 (M+H).

Using General procedure 32 and Example 562A as the appropriate indane and thieno[3,2-b]pyridin-7-ol as the appropriate alcohol, Example 562 was obtained. HRMS calculated for C₃₈H₃₇N₂O₃SCl: 636.2213; found: 637.2271 (M+H).

Using General procedure 32 and Example 563A as the appropriate indane and thieno[3,2-b]pyridin-7-ol as the appropriate alcohol, Example 563 was obtained. HRMS calculated for C₃₈H₃₇N₂O₃SCl: 636.2213; found: 637.2278 (M+H).

### Example 564 and Example 565

### Example 564A 2-[(acetyloxy)methyl]-2-hydroxyhept-3-yn-1-yl acetate

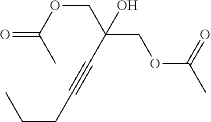 [SMILES: CCCC#CC(O)(COC(C)=O)COC(C)=O]

To a solution of n-pentyne (3.04 mL, 30.8 mmol, 1.1 eq) in THE (40 mL) cooled to 0° C. was added nBuLi solution (2.4 M in hexanes, 12.25 mL, 29.4 mmol, 1.05 eq) dropwise. The resulting solution was cooled to −78° C. and a solution of diacetoxyacetone (4.88 g, 28.0 mmol, 1 eq) in THF (15 mL) was added dropwise. After stirring at −78° C. for 30 min the reaction was quenched by the addition of AcOH (1.68 mL, 29.4 mmol, 1.05 eq) and stirred at rt for 30 min.

The mixture was partitioned between EtOAc and brine, the organic phase was separated, and the aq. phase was extracted with EtOAc. The combined organic extracts were washed with brine, dried (MgSO₄), filtered and concentrated in vacuo. Purification by automated flash chromatography (CombiFlash Rf, 80 g RediSep™ silica cartridge) eluting with a gradient of 0-40% EtOAc in heptane afforded Example 564A as a pale yellow oil (5.2 g, 21.5 mmol, 77%). ¹H NMR (400 MHz, CDCl₃) δ ppm: 4.24 (d, J=11.2 Hz, 2H), 4.18 (d, J=11.2 Hz, 2H), 2.72 (s, 1H), 2.18 (t, J=7.0 Hz, 2H), 2.12 (s, 6H), 1.57-1.46 (m, 2H), 0.97 (t, J=7.4 Hz, 3H).

### Example 564B 2-(pent-1-yn-1-yl)propane-1,3-diol

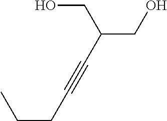

To a solution of Example 564A (4.96 g, 20.5 mmol) in DCM (30 mL), cooled to −40° C., was added TEA (8.54 mL, 61.4 mmol, 3 eq) followed by the dropwise addition of a solution of MsCl (3.17 mL, 41 mmol, 2 eq) in DCM (10 mL). The temperature was allowed to rise to −30° C. and the resulting suspension was stirred for 1 h at this temperature and then quenched with water. After 15 min the mixture was warmed to rt and diluted with brine. The organic phase was separated, and the aq. phase was extracted with DCM. The combined organic extracts were washed with brine, dried (MgSO₄) and concentrated in vacuo (low temperature) to obtain the desired intermediate that was immediately taken up in THE (40 mL) and cooled to −15° C. To this solution was added LAH (1 M in THF, 42 mL, 42 mmol, 2 eq) dropwise and stirring continued at −15° C. for 1 h and then the mixture was slowly allowed to warm to 15° C. and stirred for 18 h. Water (1.6 mL) was added dropwise, followed by the dropwise addition of 15% aq. NaOH solution (1.6 mL) and then water (4.8 mL), and the mixture was stirred for 1 h. The solids were filtered, washed with EtOAc. The filtrate was concentrated in vacuo. Purification by automated flash chromatography (CombiFlash Rf, 40 g RediSep™ silica cartridge) eluting with a gradient of 0-50% EtOAc in heptane afforded Example 564B as a yellow oil (0.98 g, 6.89 mmol, 34%). ¹H NMR (400 MHz, CDCl₃) δ ppm: 3.81-3.69 (m, 4H), 2.84-2.75 (m, 1H), 2.16 (td, J=7.1, 2.2 Hz, 2H), 2.08-1.98 (m, 2H), 1.58-1.46 (m, 2H), 0.97 (t, J=7.3 Hz, 3H).

### Example 564C 2-({[tert-butyl(dimethyl)silyl]oxy}methyl)hept-3-yn-1-ol

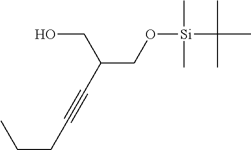 [SMILES: CCCC#CC(CO)CO[Si](C)(C)C(C)(C)C]

Using General procedure 41a and Example 564B as the appropriate alcohol and TBDMS-Cl as the appropriate silyl chloride, a racemate, Example 564C, was isolated as a colourless oil.

¹H NMR (400 MHz, CDCl₃) δ ppm: 3.85-3.65 (m, 4H), 2.80-2.71 (m, 1H), 2.50 (dd, J=7.5, 5.1 Hz, 1H), 2.13 (td, J=7.1, 2.2 Hz, 2H), 1.55-1.45 (m, 2H), 0.96 (t, J=7.4 Hz, 3H), 0.89 (s, 9H), 0.08 (s, 3H), 0.07 (s, 3H).

### Example 564D tert-butyl{[2-(iodomethyl)hept-3-yn-1-yl]oxy}dimethylsilane

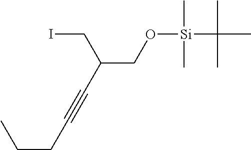 [SMILES: CCCC#CC(CI)CO[Si](C)(C)C(C)(C)C]

To a solution of Example 564C (1.0 g, 3.9 mmol, 1 eq) and PPh₃ (1.53 g, 5.85 mmol, 1.5 eq) in THE (20 mL), cooled to 0° C. was added NIS (1.32 g, 5.85 mmol, 1.5 eq) in small portions and then stirred at rt under N₂ for 18 h. The reaction was quenched by the addition of sat. aq. NH₄Cl solution, and then diluted with EtOAc. The phases were separated, and the aq. phase was extracted with EtOAc. The combined organic phases were washed with sat. aq. Na₂S₂O₃ solution, brine, dried (MgSO₄) and concentrated in vacuo. Purification by automated flash chromatography (CombiFlash Rf, 40 g RediSep™ silica cartridge) eluting with a gradient of 0-3% EtOAc in heptane afforded a racemate, Example 564D, isolated as a colourless oil (903 mg, 2.46 mmol, 63%). ¹H NMR (400 MHz, CDCl₃) δ ppm: 3.71 (dd, J=9.9, 4.7 Hz, 1H), 3.52 (dd, J=9.9, 8.2 Hz, 1H), 3.40 (dd, J=9.4, 4.8 Hz, 1H), 3.34 (dd, J=9.4, 5.7 Hz, 1H), 2.64-2.55 (m, 1H), 2.14 (td, J=7.0, 2.2 Hz, 2H), 1.57-1.47 (m, 2H), 0.99 (t, J=7.3 Hz, 3H), 0.89 (s, 9H), 0.08 (s, 3H), 0.07 (s, 3H).

### Example 564E [2-({[tert-butyl(dimethyl)silyl]oxy}methyl)hept-3-yn-1-yl](iodido)zinc

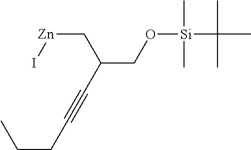

Zinc (225 mg, 3.44 mmol, 1.4 eq) was added to an oven-dried flask and heated at 150° C. under high vacuum for 30 min and then allowed to cool to rt and placed under a N₂ atmosphere. A solution of 1,2-dibromoethane (11 uL, 0.12 mmol, 0.05 eq) in THE (1 mL) was added and heated quickly with a hot air gun until bubbling occurred, followed by cooling. This process was repeated (×3) and then TMS-Cl (15 uL, 0.12 mmol, 0.05 eq) in THF (0.5 mL) was added dropwise at 25° C. and the mixture was stirred at 25° C. for 15 min. A solution of Example 564D (900 mg, 2.46 mmol, 1 eq) in THE (3.5 mL) was added dropwise at 30° C. After addition the reaction mixture was stirred at 40° C. for 6 h. After cooling and cannulation through a filter (cotton-wool/Celite/cotton-wool) into a dry Schlenk tube, a racemate, Example 564E was obtained as a colourless solution (7 mL, 1.47 mmol, 60%) with a concentration of 0.21 M, (determined by titration with a 0.5 M solution of LiCl in THE and I₂) that was used without further characterization.

### Example 564F methyl (1r,4r)-2′-[2-({[tert-butyl(dimethyl)silyl]oxy}methyl)hept-3-yn-1-yl]-4-(3-chloroanilino)spiro[cyclohexane-1,1′-indene]-4-carboxylate

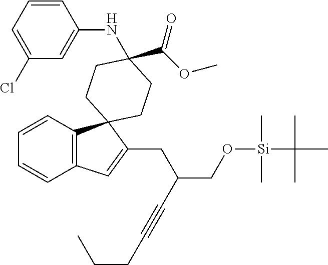 [SMILES: CCCC#CC(CO[Si](C)(C)C(C)(C)C)CC1=Cc2ccccc2[C@]12CC[C@@](Nc1cccc(Cl)c1)(C(=O)OC)CC2]

Using General Procedure 27A and Example 564E as the appropriate zinc reagent and Preparation 6c as the appropriate 2-bromo-indene derivative, a racemate, Example 564F was isolated as a solid. LRMS calculated for C₃₆H₄₈ClNO₃Si: 605; found: 606 (M+H).

### Example 564G methyl (1r,4r)-4-(3-chloroanilino)-2′-[2-(hydroxymethyl)hept-3-yn-1-yl]spiro[cyclohexane-1,1′-indene]-4-carboxylate

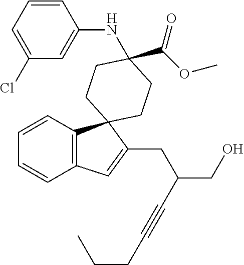 [SMILES: CCCC#CC(CO)CC1=Cc2ccccc2[C@]12CC[C@@](Nc1cccc(Cl)c1)(C(=O)OC)CC2]

Using General Procedure 29 and Example 564F as the appropriate silyl protected intermediate, a racemate, Example 564G was isolated as a yellow oil. LRMS calculated for C₃₀H₃₄ClNO₃: 491; found: 492 (M+H).

### Example 564H methyl (1r,4R)-4-(3-chloroanilino)-2′-[(2R)-2-{[(thieno[3,2-b]pyridin-7-yl)oxy]methyl}hept-3-yn-1-yl]spiro[cyclohexane-1,1′-indene]-4-carboxylate

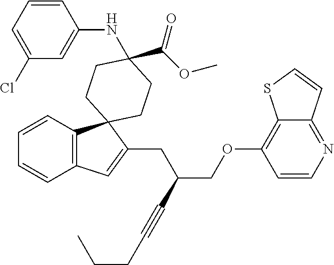 [SMILES: CCCC#C[C@H](COc1ccnc2ccsc12)CC1=Cc2ccccc2[C@]12CC[C@@](Nc1cccc(Cl)c1)(C(=O)OC)CC2]

and

### Example 5641 methyl (1r,4S)-4-(3-chloroanilino)-2′-[(2S)-2-{[(thieno[3,2-b]pyridin-7-yl)oxy]methyl}hept-3-yn-1-yl]spiro[cyclohexane-1,1′-indene]-4-carboxylate

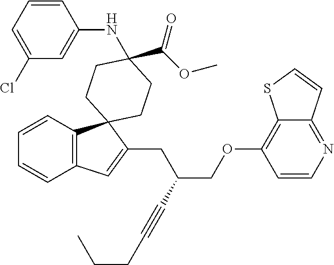 [SMILES: CCCC#C[C@@H](COc1ccnc2ccsc12)CC1=Cc2ccccc2[C@]12CC[C@@](Nc1cccc(Cl)c1)(C(=O)OC)CC2]

Using General Procedure 30a and Example 564G as the appropriate indene and thieno[2,3-b]pyridin-4-ol as the appropriate alcohol, a racemate was obtained. The enantiomers were separated by chiral chromatography. Column: AD, 50×500 mm, 20 μm, eluents: 30:70 iPrOH/heptane. The enantiomer eluting earlier was collected as Example 564H. ¹H NMR (500 MHz, DMSO-d₆) δ ppm: 8.50 (d, 1H), 7.90 (d, 1H), 7.65 (d, 1H), 7.49 (d, 1H), 7.30 (d, 1H), 7.23 (t, 1H), 7.13 (t, 1H), 7.08 (t, 1H), 7.04 (d, 1H), 6.72 (s, 1H), 6.65 (t, 1H), 6.61 (dd, 1H), 6.47 (dd, 1H), 6.45 (s, 1H), 4.37 (m, 2H), 3.68 (s, 3H), 3.41 (m, 1H), 2.64 (m, 2H), 2.39/2.10 (m, 4H), 2.18/0.89 (m, 4H), 2.06 (td, 2H), 1.32 (m, 2H), 0.81 (t, 3H). HRMS calculated for C₃₇H₃₇ClN₂O₃S: 624.2213; found: 625.2284 (M+H).

The enantiomer eluting later was collected as Example 5641. HRMS calculated for C₃₇H₃₇ClN₂O₃S: 624.2213; found: 625.2277 (M+H).

### Example 564 (1r,4R)-4-(3-chloroanilino)-2′-[(2R)-2-{[(thieno[3,2-b]pyridin-7-yl)oxy]methyl}hept-3-yn-1-yl]spiro[cyclohexane-1,1′-indene]-4-carboxylic acid

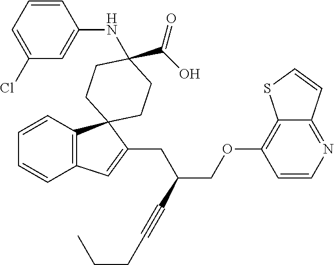 [SMILES: CCCC#C[C@H](COc1ccnc2ccsc12)CC1=Cc2ccccc2[C@]12CC[C@@](Nc1cccc(Cl)c1)(C(=O)O)CC2]

Using General procedure 33a and Example 564H as the appropriate ester, Example 564 was obtained. ¹H NMR (500 MHz, DMSO-d₆) δ ppm: 12.79 (br s, 1H), 8.50 (d, 1H), 7.90 (d, 1H), 7.66 (d, 1H), 7.49 (d, 1H), 7.30 (d, 1H), 7.23 (t, 1H), 7.12 (t, 1H), 7.08 (t, 1H), 7.04 (d, 1H), 6.72 (br s, 1H), 6.64 (t, 1H), 6.58 (dd, 1H), 6.56 (dd, 1H), 6.35 (br s, 1H), 4.37 (m, 2H), 3.40 (m, 1H), 2.66/2.54 (dd+m, 2H), 2.39-0.88 (m, 8H), 2.06 (td, 2H), 1.32 (m, 2H), 0.81 (t, 3H). HRMS calculated for C₃₆H₃₅N₂O₃SCl: 610.2057; found: 611.2140 (M+H).

### Example 565 (1r,4S)-4-(3-chloroanilino)-2′-[(2S)-2-{[(thieno[3,2-b]pyridin-7-yl)oxy]methyl}hept-3-yn-1-yl]spiro[cyclohexane-1,1′-indene]-4-carboxylic acid

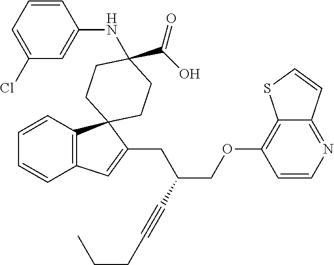 [SMILES: CCCC#C[C@@H](COc1ccnc2ccsc12)CC1=Cc2ccccc2[C@]12CC[C@@](Nc1cccc(Cl)c1)(C(=O)O)CC2]

Using General procedure 33a and Example 5641 as the appropriate ester, Example 565 was obtained. HRMS calculated for C₃₆H₃₅N₂O₃SCl: 610.2057; found: 611.2116 (M+H).

### Example 566

### Example 566A 2-(pyridin-2-yl)prop-2-en-1-ol

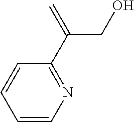

To a solution of 2-(pyridin-2-yl)propane-1,3-diol (1.58 g, 10.3 mmol, 1 eq) in DCM (30 mL) was added TEA (3.3 mL, 23.7 mmol, 2.3 eq) and the mixture was cooled to 0° C. MsCl (1.71 mL, 22.1 mmol, 2.15 eq) was added dropwise and the mixture was stirred at 0° C. for 1.5 h. Then it was concentrated in vacuo and the residue was dissolved in water (24 mL) and 1,4-dioxane (24 mL). NaOH (2.06 g, 51.5 mmol, 5 eq) was added and the mixture was heated at 80° C. for 24 h, cooled to rt and the pH was adjusted to 7 with 2 M aq. HCl solution. The mixture was extracted with EtOAc and the combined organic extracts were washed with brine, dried (MgSO₄) and concentrated in vacuo. Purification by automated flash chromatography (CombiFlash Rf, 40 g RediSep™ silica cartridge) eluting with a gradient of 0-5% MeOH in DCM afforded Example 566A as a yellow oil (708 mg, 4.4 mmol, 43%). LRMS calculated for C₈H₉NO: 135; found: 136 (M+H).

### Example 566B 2-{3-[(4-methoxyphenyl)methoxy]prop-1-en-2-yl}pyridine

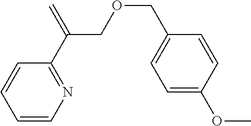 [SMILES: C=C(COCc1ccc(OC)cc1)c1ccccn1]

To a solution of Example 566A (590 mg, 4.37 mmol, 1 eq) in THE (25 mL) cooled to 0° C. was added NaH (60% dispersion in mineral oil; 168 mg, 6.98 mmol, 1.6 eq) in portions, followed by TBAI (81 mg, 0.22 mmol, 0.05 eq) and the mixture was stirred at 0° C. for 30 min. PMB-Br (0.66 mL, 4.58 mmol, 1.05 eq) was added dropwise and stirring continued at 0° C. for 2 h. The mixture was partitioned between EtOAc and water. The phases were separated, and the organic phase was washed with sat. aq. NaHCO₃ solution, brine, dried (MgSO₄) and concentrated in vacuo. Purification by automated flash chromatography (CombiFlash Rf, 24 g RediSep™ silica cartridge) eluting with a gradient of 0-65% EtOAc in heptane afforded Example 566B as a colourless oil (707 mg, 2.77 mmol, 63%). LRMS calculated for C₁₆H₁₇NO₂: 255; found: 256 (M+H).

### Example 566C methyl (1r,4r)-4-(3-chloroanilino)-2′-[(1E)-3-[(4-methoxyphenyl)methoxy]-2-(pyridin-2-yl)prop-1-en-1-yl]spiro[cyclohexane-1,1′-indene]-4-carboxylate

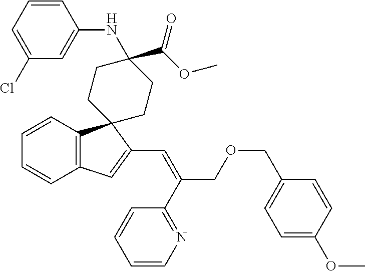 [SMILES: COc1ccc(COC/C(=C/C2=Cc3ccccc3[C@]23CC[C@@](Nc2cccc(Cl)c2)(C(=O)OC)CC3)c2ccccn2)cc1]

To an oven-dried flask was added Example 566B (505 mg, 1.98 mmol, 1 eq), Preparation 6c (1.06 g, 2.37 mmol, 1.2 eq), tris(2-methylphenyl)phosphane (120 mg, 0.4 mmol, 0.2 eq), TBAB (638 mg, 1.98 mmol, 1 eq), Pd(OAc)₂ (22 mg, 0.1 mmol, 0.05 eq) and DMF (10 mL). TEA (0.41 mL, 2.97 mmol, 1.5 eq) was added and the mixture was sparged with N₂ before heating at 100° C. for 90 h. Further Pd(OAc)₂ (22 mg, 0.1 mmol, 0.05 eq), tris(2-methylphenyl)phosphane (39 mg, 0.13 mmol, 0.06 eq) and TEA (0.41 mL, 2.97 mmol, 1.5 eq) in DMF (1 mL) were added and the mixture was heated at 110° C. for 24 h. The mixture was partitioned between EtOAc and water, the phases were separated, and the organic phase was washed with sat. aq. NaHCO₃ solution, brine, dried (MgSO₄) and concentrated in vacuo. Purification by automated flash chromatography (CombiFlash Rf, 80 g RediSep™ silica cartridge) eluting with a gradient of 0-35% EtOAc in heptane afforded Example 566C as a yellow solid (230 mg, 0.37 mmol, 16%). LRMS calculated for C₃₈H₃₇ClN₂O₄Si: 620; found: 621 (M+H).

### Example 566D methyl (1r,4r)-4-(3-chloroanilino)-2′-{3-[(4-methoxyphenyl)methoxy]-2-(pyridin-2-yl)propyl}spiro[cyclohexane-1,1′-indene]-4-carboxylate

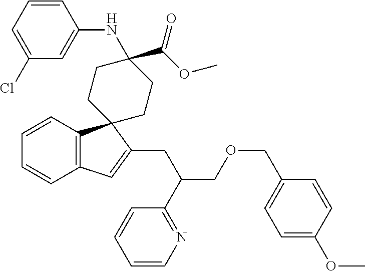 [SMILES: COc1ccc(COCC(CC2=Cc3ccccc3[C@]23CC[C@@](Nc2cccc(Cl)c2)(C(=O)OC)CC3)c2ccccn2)cc1]

To a solution of Example 566C (230 mg, 0.37 mmol, 1 eq) in MeOH (8 mL) and EtOAc (16 mL) was added Pt/C (7 mg) under N₂. The mixture was evacuated and backfilled with N₂ (×3), then evacuated and backfilled with H₂ and stirred at 50° C. for 48 h under an atmosphere of H₂.

The mixture was filtered through celite, washed with MeOH and the filtrate was concentrated under reduced pressure. Purification by reverse phase automated flash chromatography (CombiFlash Rf, C18 26 g RediSep column) eluting with a gradient of 10-100% MeCN in water afforded a racemate, Example 566D, isolated as a yellow solid (156 mg, 0.25 mmol, 68%). LRMS calculated for C₃₈H₃₉ClN₂O₄: 622; found: 623 (M+H).

### Example 566E methyl (1r,4r)-4-(3-chloroanilino)-2′-[3-hydroxy-2-(pyridin-2-yl)propyl]spiro[cyclohexane-1,1′-indene]-4-carboxylate

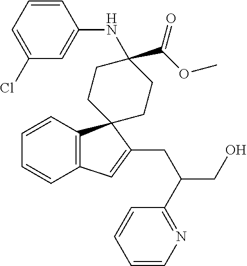 [SMILES: COC(=O)[C@]1(Nc2cccc(Cl)c2)CC[C@]2(CC1)C(CC(CO)c1ccccn1)=Cc1ccccc12]

To a solution of Example 566D (102 mg, 0.16 mmol, 1 eq) in MeCN (12 mL) was added 2-bromo-2-methylpropane (0.05 mL, 0.41 mmol, 2.5 eq) and the mixture was heated at reflux for 2 h. The mixture was cooled to rt and concentrated in vacuo. The residue was partitioned between EtOAc and water, and the organic phase was washed with sat. aq. NaHCO₃ solution, brine, dried (MgSO₄) and concentrated in vacuo. Purification by automated flash chromatography (CombiFlash Rf, 40 g RediSep™ silica cartridge) eluting with a gradient of 0-5% MeOH in DCM afforded a racemate, Example 566E, isolated as a white solid (45 mg, 0.09 mmol, 55%). LRMS calculated for C₃₀H₃1ClN₂O₃: 502; found: 503 (M+H).

### Example 566F methyl (1r,4R)-4-(3-chloroanilino)-2′-[3-{[(5R)-5-methyl-5,6,7,8-tetrahydroquinolin-4-yl]oxy}-2-(pyridin-2-yl)propyl]spiro[cyclohexane-1,1′-indene]-4-carboxylate

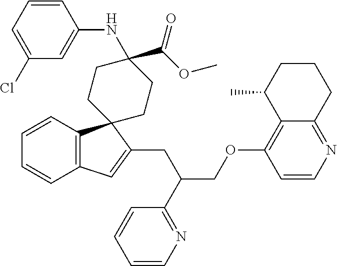 [SMILES: COC(=O)[C@]1(Nc2cccc(Cl)c2)CC[C@]2(CC1)C(CC(COc1ccnc3c1[C@H](C)CCC3)c1ccccn1)=Cc1ccccc12]

Using General Procedure 30a and Example 566E as the appropriate indene and Preparation 2a1 as the appropriate alcohol, a mixture of diastereoisomers, Example 566F, was isolated as a colourless oil. LRMS calculated for C₄₀H₄₂ClN₃O₃: 647; found: 648 (M+H).

### Example 566 (1r,4R)-4-(3-chloroanilino)-2′-[3-{[(5R)-5-methyl-5,6,7,8-tetrahydroquinolin-4-yl]oxy}-2-(pyridin-2-yl)propyl]spiro[cyclohexane-1,1′-indene]-4-carboxylic acid

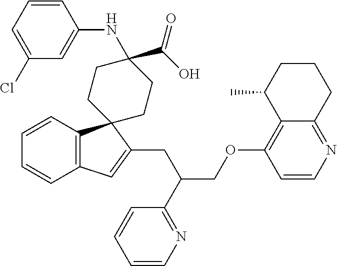 [SMILES: C[C@@H]1CCCc2nccc(OCC(CC3=Cc4ccccc4[C@]34CC[C@@](Nc3cccc(Cl)c3)(C(=O)O)CC4)c3ccccn3)c21]

To a solution of Example 566F (25 mg, 0.04 mmol, 1 eq) in MeOH (5 mL) was added LiOH×H₂O (8 mg, 0.19 mmol, 5 eq) and the mixture was heated at 110° C. for 1 h under microwave irradiation. The reaction was allowed to cool to rt and the pH was adjusted to 4 using 2 M aq. HCl solution and partitioned between EtOAc and water. The organic phase was washed with brine, dried (MgSO₄) and concentrated in vacuo. Purification by reverse phase automated flash chromatography (CombiFlash Rf, C18 5.5 g Gold RediSep column) eluting with a gradient of 10-100% MeCN in water afforded a mixture of diastereoisomers, Example 566, isolated as a white solid (13.1 mg, 0.02 mmol, 54%). LRMS calculated for C₃₉H₄₀ClN₃O₃: 633; found: 634 (M+H). ¹H NMR (400 MHz, DMSO-d₆) δ ppm: 12.80 (br s, 1H), 8.53-8.47 (m, 1H), 8.16-8.10 (m, 1H), 7.75-7.67 (m, 1H), 7.67-7.60 (m, 1H), 7.45-7.39 (m, 1H), 7.24-7.15 (m, 3H), 7.14-7.05 (m, 2H), 6.81 (d, J=5.7 Hz, 1H), 6.69-6.64 (m, 1H), 6.62-6.53 (m, 2H), 6.47-6.32 (m, 2H), 4.44-4.26 (m, 2H), 3.86-3.74 (m, 1H), 3.00-2.54 (m, 5H), 2.48-2.04 (m, 6H), 1.83-1.44 (m, 4H), 1.01-0.63 (m, 5H).

### Example 567 (1r,4R)-4-(3-chloroanilino)-2′-[(2R)-2-methyl-3-phenoxypropyl]-2′,3′-dihydrospiro[cyclohexane-1,1′-indene]-4-carboxylic acid

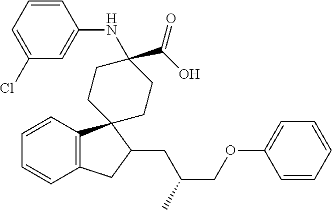

Using General procedure 30b and phenol (35 mg, 0.37 mmol, 1.5 eq) as the appropriate alcohol and Preparation 10b (110 mg, 0.25 mmol, 1 eq) as the appropriate indane, the desired intermediate was obtained. Using General procedure 33b a mixture of diastereoisomers, Example 567 was obtained as a white powder (10.2 mg, 0.02 mmol, 8%). LRMS calculated for C₃₁H₃₄ClNO₃: 503; found: 504 (M+H). ¹H NMR (400 MHz, DMSO-d₆) δ ppm: 7.42-7.35 (m, 1H), 7.30-7.11 (m, 5H), 7.08-7.02 (m, 1H), 6.97-6.87 (m, 3H), 6.63 (t, J=2.1 Hz, 1H), 6.58-6.52 (m, 2H), 3.96-3.76 (m, 2H), 3.06-2.92 (m, 1H), 2.64-2.37 (m, 2H), 2.18-1.62 (m, 7H), 1.54-1.14 (m, 4H), 1.06/1.02 (d, J=6.7 Hz, 3H).

### Example 568 (1r,2′S,4S)-4-(3-chloroanilino)-2′-{(2R)-2-methyl-3-[(pyridin-3-yl)oxy]propyl}-2′,3′-dihydrospiro[cyclohexane-1,1′-indene]-4-carboxylic acid

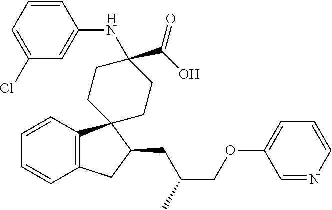 [SMILES: C[C@@H](COc1cccnc1)C[C@H]1Cc2ccccc2[C@]12CC[C@@](Nc1cccc(Cl)c1)(C(=O)O)CC2]

Using General procedure 31c and Preparation 10b1 (100 mg, 0.23 mmol, 1 eq) as the appropriate alcohol and 3-chloropyridine (50 μL, 0.52 mmol, 2.3 eq) as the appropriate aryl chloride, Example 568 was obtained as an orange powder (11.9 mg, 0.02 mmol, 10%). LRMS calculated for C₃₀H₃₃ClN₂O₃: 504; found: 505 (M+H). ¹H NMR (400 MHz, DMSO-d₆) δ ppm: 8.28 (d, J=2.9 Hz, 1H), 8.16 (dd, J=4.5, 1.5 Hz, 1H), 7.41-7.35 (m, 2H), 7.31 (dd, J=8.5, 4.5 Hz, 1H), 7.24-7.11 (m, 3H), 7.06 (t, J=8.1 Hz, 1H), 6.63 (t, J=2.1 Hz, 1H), 6.58-6.52 (m, 2H), 3.96-3.85 (m, 2H), 2.98 (dd, J=15.6, 7.2 Hz, 1H), 2.59-2.39 (m, 2H), 2.20-1.83 (m, 6H), 1.79-1.67 (m, 1H), 1.54-1.27 (m, 4H), 1.03 (d, J=6.7 Hz, 3H).

### Example 569 (1r,2′S,4S)-4-(3-chloroanilino)-2′-{(2R)-2-methyl-3-[3-(methylamino)phenoxy]propyl}-2′,3′-dihydrospiro[cyclohexane-1,1′-indene]-4-carboxylic acid

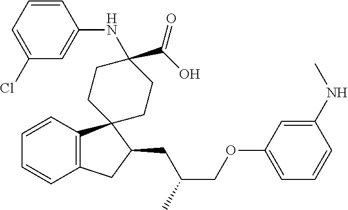 [SMILES: CNc1cccc(OC[C@H](C)C[C@H]2Cc3ccccc3[C@]23CC[C@@](Nc2cccc(Cl)c2)(C(=O)O)CC3)c1]

Using General procedure 32 and Preparation 10b1 as the appropriate indane and 3-(methylamino)phenol as the appropriate alcohol Example 569 was obtained. ¹H NMR (500 MHz, DMSO-d₆) δ ppm: 7.38 (dm, 1H), 7.21 (dm, 1H), 7.15 (m, 2H), 7.05 (tm, 1H), 6.93 (tm, 1H), 6.62 (t, 1H), 6.54 (m, 2H), 6.11 (dm, 1H), 6.09 (dm, 1H), 6.04 (t, 1H), 5.56 (br s, 1H), 3.74/3.71 (t+t, 2H), 2.97/2.53 (dd, 2H), 2.62 (s, 3H), 2.15-1.32 (m, 8H), 2.11 (m, 1H), 1.92 (m, 1H), 1.43/1.28 (t+t, 1H), 1.00 (d, 3H). HRMS calculated for C₃₂H₃₇N₂O₃Cl: 532.2493; found: 533.2562 (M+H).

### Example 570 and Example 571 and Example 572 and Example 573

### Example 570A 2-(1,4-Dioxaspiro[4.5]decan-8-yl)propan-1-ol

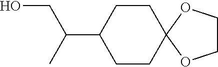

Using General procedure 1 and ethyl 2-(1,4-dioxaspiro[4.5]decan-8-yl)propanoate (4.85 g, 20 mmol, 1.0 eq.) as the appropriate ester and LAH (0.76 g, 20 mmol, 1.0 eq.) at rt the crude alcohol was obtained. Then it was purified via flash chromatography using heptane and EtOAc as eluents to yield Example 570A (3.61 g, 18 mmol, 90%). MS (EI, 70 eV) m/z (% relative intensity, [ion]): 55 (12), 86 (14), 99 (100), 141 (5), 182 (2), 200 (1, [M⁺]).

### Example 570B 8-(1-bromopropan-2-yl)-1,4-dioxaspiro[4.5]decane

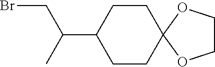

Using General procedure 3 and Example 570A (3.61 g, 18 mmol, 90%). as the appropriate alcohol Example 570B (3.79 g, 14.4 mmol, 80%) was obtained. ¹H NMR (400 MHz, CDCl₃) δ ppm: 3.95-3.90 (m, 4H), 3.47-3.41 (m, 1H), 3.40-3.35 (m, 1H), 1.80-1.73 (m, 2H), 1.73-1.62 (m, 3H), 1.59-1.46 (m, 2H), 1.46-1.39 (m, 1H), 1.39-1.20 (m, 2H), 1.01 (d, 3H).

### Example 570C bromido[2-(1,4-dioxaspiro[4.5]decan-8-yl)propyl]zinc

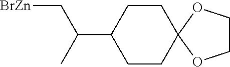

Using General procedure 4 and Example 570B (3.79 g, 14.4 mmol) as the appropriate bromo compound Example 570C was obtained, which was directly used in the next step.

### Example 570D methyl (1r,4r)-4-(3-chloroanilino)-2′-[2-(1,4-dioxaspiro[4.5]decan-8-yl)propyl]spiro[cyclohexane-1,1′-indene]-4-carboxylate, enantiomer 1

and

### Example 572D methyl (1r,4r)-4-(3-chloroanilino)-2′-[2-(1,4-dioxaspiro[4.5]decan-8-yl)propyl]spiro[cyclohexane-1,1′-indene]-4-carboxylate, enantiomer 2

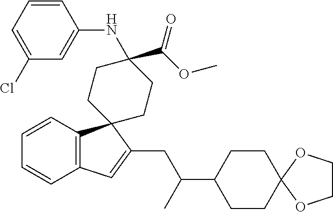 [SMILES: COC(=O)[C@]1(Nc2cccc(Cl)c2)CC[C@]2(CC1)C(CC(C)C1CCC3(CC1)OCCO3)=Cc1ccccc12]

Using General procedure 27a and Preparation 6c as the appropriate 2-bromo-indene derivative and Example 570C as the appropriate Zn reagent a mixture of enantiomers was obtained. ¹H NMR (500 MHz, DMSO-d₆) δ ppm: 7.64 (dm, 1H), 7.27 (dm, 1H), 7.21 (m, 1H), 7.10 (m, 1H), 7.09 (t, 1H), 6.65 (t, 1H), 6.59 (dm, 1H), 6.46 (dm, 1H), 6.45 (m, 1H), 6.44 (s, 1H), 3.86-3.82 (m, 4H), 3.70 (s, 3H), 2.45-0.74 (m, 8H), 2.32/1.96 (m+m, 2H), 1.75-1.25 (m, 8H), 1.78 (m, 1H), 1.32 (m, 1H), 0.85 (d, 3H). HRMS calculated for C₃₃H₄₀ClNO₄: 549.2646; found: 550.2717 (M+H).

The isomers were separated by chiral chromatography. Column: AD, 50×500 mm, 20 μm, Eluent: EtOH. The enantiomer eluting earlier was collected as Example 570D. The enantiomer eluting later was collected as Example 572D.

### Example 570E methyl (1r,4r)-4-(3-chloroanilino)-2′-[2-(1,4-dioxaspiro[4.5]decan-8-yl)propyl]spiro[cyclohexane-1,1′-indene]-4-carboxylate, diastereoisomer 1 enantiomer 1

and

### Example 571E methyl (1r,4r)-4-(3-chloroanilino)-2′-[2-(1,4-dioxaspiro[4.5]decan-8-yl)propyl]spiro[cyclohexane-1,1′-indene]-4-carboxylate, diastereoisomer 2 enantiomer 1

and

### Example 572E methyl (1r,4r)-4-(3-chloroanilino)-2′-[2-(1,4-dioxaspiro[4.5]decan-8-yl)propyl]spiro[cyclohexane-1,1′-indene]-4-carboxylate, diastereoisomer 1 enantiomer 2

and

### Example 573E methyl (1r,4r)-4-(3-chloroanilino)-2′-[2-(1,4-dioxaspiro[4.5]decan-8-yl)propyl]spiro[cyclohexane-1,1′-indene]-4-carboxylate, diastereoisomer 2 enantiomer 2

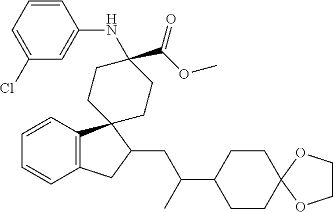

Using General procedure 19 and Example 570D as the appropriate indene derivative a mixture of diastereoisomers was obtained, which was purified via flash chromatography using heptane and EtOAc as eluents. The diastereoisomers were separated by chiral chromatography. Column: AD, 50×500 mm, 20 μm, Eluents: heptane/EtOH 9:1. The diastereoisomer eluting earlier was collected as Example 570E. HRMS calculated for C₃₃H₄₂ClNO₄: 551.2802; found: 552.2889 (M+H).

The diastereoisomer eluting later was collected as Example 571E. HRMS calculated for C₃₃H₄₂ClNO₄: 551.2802; found: 552.2883 (M+H).

Using General procedure 19 and Example 572D as the appropriate indene derivative a mixture of diastereoisomers was obtained. The diastereoisomers were separated by chiral chromatography. Column: AD, 50×500 mm, 20 μm, Eluents: heptane/EtOH 1:1. The diastereoisomer eluting earlier was collected as Example 572E. HRMS calculated for C₃₃H₄₂ClNO₄: 551.2802; found: 552.2855 (M+H).

The diastereoisomer eluting later was collected as Example 573E. HRMS calculated for C₃₃H₄₂ClNO₄: 551.2802; found: 552.2860 (M+H).

### Example 570 (1r,4r)-4-(3-chloroanilino)-2′-[2-(4-oxocyclohexyl)propyl]-2′,3′-dihydrospiro[cyclohexane-1,1′-indene]-4-carboxylic acid, diastereoisomer 1 enantiomer 1

and

### Example 571 (1r,4r)-4-(3-chloroanilino)-2′-[2-(4-oxocyclohexyl)propyl]-2′,3′-dihydrospiro[cyclohexane-1,1′-indene]-4-carboxylic acid, diastereoisomer 2 enantiomer 1

and

### Example 572 (1r,4r)-4-(3-chloroanilino)-2′-[2-(4-oxocyclohexyl)propyl]-2′,3′-dihydrospiro[cyclohexane-1,1′-indene]-4-carboxylic acid, diastereoisomer 1 enantiomer 2

and

### Example 573 (1r,4r)-4-(3-chloroanilino)-2′-[2-(4-oxocyclohexyl)propyl]-2′,3′-dihydrospiro[cyclohexane-1,1′-indene]-4-carboxylic acid, diastereoisomer 2 enantiomer 2

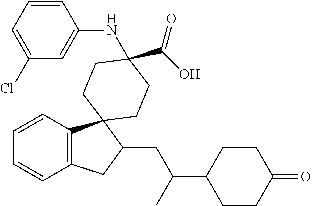

Using General procedure 33a and Example 570E as the appropriate ester a crude reaction mixture was obtained, which was acidified to pH=3 with 2 M aq HCl solution, then silica-gel was added and the volatiles were evaporated under reduced pressure. Then it was purified via flash chromatography using DCM and MeOH as eluents and further purified via prep RP-HPLC using 25 mM aq. NH₄HCO₃ solution and MeCN as eluents which resulted in Example 570. ¹H NMR (500 MHz, DMSO-d₆) δ ppm: 7.40-7.10 (m, 4H), 7.06 (t, 1H), 6.64 (t, 1H), 6.55 (dm, 1H), 6.53 (dm, 1H), 6.20 (br s, 1H), 2.96/2.50 (dd+dd, 2H), 2.46-1.30 (m, 8H), 2.37/2.19 (m+m, 4H), 2.04 (m, 1H), 1.94-1.81/1.49-1.38 (m+m, 4H), 1.65 (m, 1H), 1.52 (m, 1H), 1.26 (m, 2H), 0.89 (d, 3H). HRMS calculated for C₃₀H₃₆NO₃Cl: 493.2384; found: 494.2450 (M+H).

Using General procedure 33a and Example 571E as the appropriate ester a crude reaction mixture was obtained, which was acidified to pH=3 with 2 M aq HCl solution, then silica-gel was added and the volatiles were evaporated under reduced pressure. Then it was purified via flash chromatography using DCM and MeOH as eluents and further purified via prep RP-HPLC using 25 mM aq. NH₄HCO₃ solution and MeCN as eluents which resulted in Example 571. ¹H NMR (500 MHz, DMSO-d₆) δ ppm: 7.47-7.10 (m, 4H), 7.06 (t, 1H), 6.61 (t, 1H), 6.55 (dm, 1H), 6.52 (dm, 1H), 6.24 (br s, 1H), 2.96/2.56 (dd+dd, 2H), 2.48-2.14 (m, 4H), 2.48-1.17 (m, 8H), 2.03 (m, 1H), 2.00/1.84/1.38 (br/m+qd, 4H), 1.84 (m, 1H), 1.66 (br, 1H), 1.58/1.13 (m+m, 2H), 0.87 (d, 3H). HRMS calculated for C₃₀H₃₆NO₃Cl: 493.2384; found: 494.2461 (M+H).

Using General procedure 33a and Example 572E as the appropriate ester a crude reaction mixture was obtained, which was acidified to pH=3 with 2 M aq HCl solution, then silica-gel was added and the volatiles were evaporated under reduced pressure. Then it was purified via flash chromatography using DCM and MeOH as eluents and further purified via prep RP-HPLC using 25 mM aq. NH₄HCO₃ solution and MeCN as eluents which resulted in Example 572. HRMS calculated for C₃₀H₃₆NO₃Cl: 493.2384; found: 494.2454 (M+H).

Using General procedure 33a and Example 573E as the appropriate ester a crude reaction mixture was obtained, which was acidified to pH=3 with 2 M aq HCl solution, then silica-gel was added and the volatiles were evaporated under reduced pressure. Then it was purified via flash chromatography using DCM and MeOH as eluents and further purified via prep RP-HPLC using 25 mM aq. NH₄HCO₃ solution and MeCN as eluents which resulted in Example 573. HRMS calculated for C₃₀H₃₆NO₃Cl: 493.2384; found: 494.2461 (M+H).

### Example 574 (1r,2′R,4R)-4-(3-chloroanilino)-2′-{(2R)-2-methyl-3-[(pyridin-4-yl)amino]propyl}-2′,3′-dihydrospiro[cyclohexane-1,1′-indene]-4-carboxylic acid

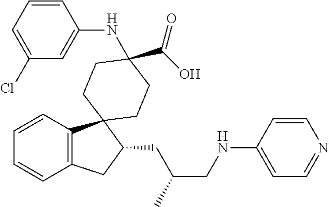 [SMILES: C[C@@H](CNc1ccncc1)C[C@@H]1Cc2ccccc2[C@]12CC[C@@](Nc1cccc(Cl)c1)(C(=O)O)CC2]

Using General procedure 32 and Preparation 10b2 as the appropriate ester and tert-butyl N-(4-pyridyl)carbamate as the appropriate amine resulted in an intermediate, which was further treated with TFA (30 eq.) in DCM (0.05M). The crude mixture was neutralized by the addition of TEA, then it was concentrated under reduced pressure and then purified via prep RP-HPLC using 25 mM aq. NH₄HCO₃ solution and MeCN as eluents to obtain Example 574. ¹H NMR (500 MHz, DMSO-d₆) δ ppm: 12.71 (br s, 1H), 7.99 (m, 2H), 7.42-7.10 (m, 4H), 7.05 (t, 1H), 6.62 (t, 1H), 6.55 (dm, 1H), 6.54 (dm, 1H), 6.51 (m, 3H), 6.16 (br s, 1H), 3.18/2.80 (m+m, 2H), 3.00/2.57 (dd+dd, 2H), 2.50-1.23 (m, 8H), 2.10 (m, 1H), 1.82 (m, 1H), 1.50/1.18 (m+m, 2H), 0.98 (d, 3H). HRMS calculated for C₃₀H₃₄N₃O₂Cl: 503.2339; found: 504.2413 (M+H).

### Example 575 (1r,2′S,4S)-4-(3-chloroanilino)-2′-{(2R)-2-methyl-3-[(pyridin-4-yl)amino]propyl}-2′,3′-dihydrospiro[cyclohexane-1,1′-indene]-4-carboxylic acid

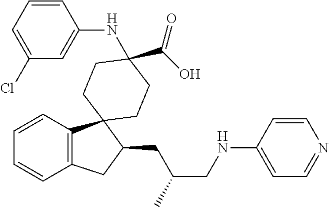 [SMILES: C[C@@H](CNc1ccncc1)C[C@H]1Cc2ccccc2[C@]12CC[C@@](Nc1cccc(Cl)c1)(C(=O)O)CC2]

Using General procedure 32 and Preparation 10b1 as the appropriate ester and tert-butyl N-(4-pyridyl)carbamate as the appropriate amine resulted in an intermediate, which was further treated with TFA (30 eq.) in DCM (0.05M). The crude mixture was neutralized by the addition of TEA, then it was concentrated under reduced pressure and then purified via prep RP-HPLC using 25 mM aq. NH₄HCO₃ solution and MeCN as eluents to obtain Example 575. ¹H NMR (500 MHz, DMSO-d₆) δ ppm: 12.79 (br s, 1H), 7.98 (m, 2H), 7.40-7.09 (m, 4H), 7.05 (t, 1H), 6.68 (t, 1H), 6.63 (t, 1H), 6.54 (dm, 1H), 6.53 (dm, 1H), 6.48 (m, 2H), 6.20 (br s, 1H), 2.95/2.92 (m+m, 2H), 2.91/2.47 (dd+dd, 2H), 2.50-1.30 (m, 8H), 2.10 (m, 1H), 1.77 (m, 1H), 1.38/1.19 (m+m, 2H), 0.95 (d, 3H). HRMS calculated for C₃₀H₃₄N₃O₂Cl: 503.2339; found: 504.2428 (M+H).

### Example 576 (1r,4R)-4-(3-chloroanilino)-2′-{(2R)-2-methyl-3-[(3-methylpyridin-4-yl)oxy]propyl}-2′,3′-dihydrospiro[cyclohexane-1,1′-indene]-4-carboxylic acid

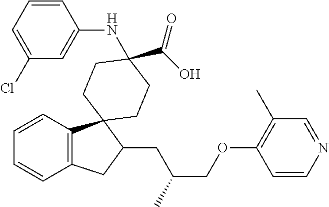

Using General procedure 31a and Preparation 10b (90 mg, 0.2 mmol, 1 eq) as the appropriate alcohol and 4-choro-3-methylpyridine (40 mg, 0.24 mmol, 1.2 eq) as the appropriate aryl chloride, the desired intermediate was obtained. Using General procedure 33b, a mixture of diastereoisomers, Example 576 was obtained as a white powder (1.4 mg, 2.7 μmol, 1%). LRMS calculated for C₃₁H₃₅N₂O₃Cl: 518; found: 519 (M+H). ¹H NMR (400 MHz, DMSO-d₆) δ ppm: 8.29-8.24 (m, 1H), 8.20/8.16 (s, 1H), 7.43-7.36 (m, 1H), 7.23-7.10 (m, 3H), 7.06-7.00 (m, 1H), 6.97-6.93 (m, 1H), 6.64/6.62 (t, J=2.1 Hz, 1H), 6.57-6.49 (m, 2H), 4.04-3.87 (m, 2H), 3.06-2.95 (m, 1H), 2.64-2.36 (m, 2H), 2.18-1.63 (m, 10H), 1.53-1.18 (m, 4H), 1.09/1.05 (d, J=6.7 Hz, 3H).

### Example 577

### Example 577A 4-chloro-3-cyclopropylpyridine

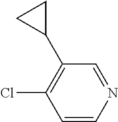

To an oven-dried flask was added 3-bromo-4-chloropyridine (520 mg, 2.7 mmol, 1 eq), potassium cyclopropyltrifluoroborate (440 mg, 2.97 mmol, 1.1 eq) and Cs₂CO₃ (2.2 g, 6.76 mmol, 2.5 eq) in toluene (11.5 mL) and water (1.5 mL). The mixture was sparged with N₂ (10 min), butyldi-1-adamantylphosphine (48 mg, 0.14 mmol, 0.05 eq) and Pd(OAc)₂ (24 mg, 0.11 mmol, 0.04 eq) were added and then the mixture was heated at 100° C. for 18 h. The mixture was allowed to cool to rt, partitioned between DCM and sat. aq. NaHCO₃ solution, and the organic phase was washed with brine, dried (MgSO₄) and concentrated in vacuo. Purification by automated flash chromatography (CombiFlash Rf, 24 g RediSep™ silica cartridge) eluting with a gradient of 0-5% MeOH in DCM afforded Example 577A as a yellow oil (216 mg, 1.4 mmol, 52%). LRMS calculated for C₈H₈ClN: 153; found: 154 (M+H). ¹H NMR (400 MHz, DMSO-d₆) δ ppm: 8.35 (d, J=5.2 Hz, 1H), 8.28 (s, 1H), 7.50 (d, J=5.2 Hz, 1H), 2.13-2.04 (m, 1H), 1.07-1.01 (m, 2H), 0.87-0.82 (m, 2H).

### Example 577 (1r,2′S,4S)-4-(3-chloroanilino)-2′-{(2R)-3-[(3-cyclopropylpyridin-4-yl)oxy]-2-methylpropyl}-2′,3′-dihydrospiro[cyclohexane-1,1′-indene]-4-carboxylic acid

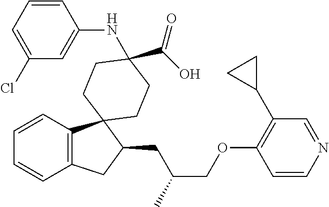 [SMILES: C[C@@H](COc1ccncc1C1CC1)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and Example 577A (120 mg, 0.78 mmol, 2.3 eq) as the appropriate aryl chloride, Example 577 was obtained as an off-white powder (108 mg, 0.2 mmol, 58%). LRMS calculated for C₃₃H₃₇ClN₂O₃: 544; found: 545 (M+H). ¹H NMR (400 MHz, DMSO-d₆) δ ppm: 8.23 (d, J=5.6 Hz, 1H), 8.00 (s, 1H), 7.40-7.34 (m, 1H), 7.21-7.11 (m, 3H), 7.05 (t, J=8.1 Hz, 1H), 6.93 (d, J=5.7 Hz, 1H), 6.63 (t, J=2.1 Hz, 1H), 6.57-6.52 (m, 2H), 3.97-3.88 (m, 2H), 3.01 (dd, J=15.6, 7.1 Hz, 1H), 2.60-2.39 (m, 2H), 2.21-2.09 (m, 2H), 2.08-1.95 (m, 2H), 1.94-1.82 (m, 3H), 1.80-1.69 (m, 1H), 1.54-1.30 (m, 4H), 1.06 (d, J=6.7 Hz, 3H), 0.89-0.61 (m, 4H).

### Example 578 (1r,2′S,4S)-4-(3-chloroanilino)-2′-[(2R)-2-methyl-3-{[3-(trifluoromethyl)pyridin-4-yl]oxy}propyl]-2′,3′-dihydrospiro[cyclohexane-1,1′-indene]-4-carboxylic acid

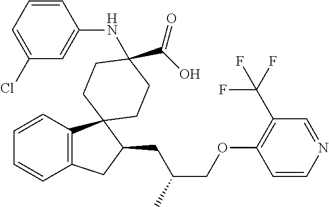 [SMILES: C[C@@H](COc1ccncc1C(F)(F)F)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and 4-chloro-3-(trifluoromethyl)pyridine×HCl (170 mg, 0.78 mmol, 2.3 eq) as the appropriate aryl chloride, Example 578 was obtained as a beige powder (44.5 mg, 0.08 mmol, 23%). LRMS calculated by C₃₁H₃₂ClF₃N₂O₃: 572; found: 573 (M+H). ¹H NMR (400 MHz, DMSO-d₆) δ ppm: 8.70-8.66 (m, 2H), 7.42-7.36 (m, 1H), 7.32 (d, J=5.9 Hz, 1H), 7.23-7.12 (m, 3H), 7.05 (t, J=8.1 Hz, 1H), 6.62 (t, J=2.1 Hz, 1H), 6.57-6.51 (m, 2H), 4.15-4.02 (m, 2H), 3.00 (dd, J=15.7, 7.2 Hz, 1H), 2.59-2.39 (m, 2H), 2.18-1.82 (m, 6H), 1.78-1.67 (m, 1H), 1.53-1.29 (m, 4H), 1.04 (d, J=6.6 Hz, 3H).

### Example 579

### Example 579A 2-({[tert-butyl(dimethyl)silyl]oxy}methyl)-4-chloropyridine

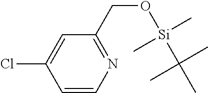 [SMILES: CC(C)(C)[Si](C)(C)OCc1cc(Cl)ccn1]

To a solution of 4-chloro-2-hydroxymethylpyridine (285 mg, 1.99 mmol, 1 eq) and DIPEA (1.04 mL, 5.96 mmol, 3 eq) in DCM (6 mL), cooled to 0° C., was added tert-butyldimethylsilyl trifluoromethanesulfonate (1.14 mL, 4.96 mmol, 2.5 eq) dropwise and the mixture was allowed to warm to rt and stirred for 18 h. The mixture was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-30% EtOAc in heptane afforded Example 579A as a colorless oil (474 mg, 1.84 mmol, 93%). LRMS calculated for C₁₂H₂₀NOSi: 257; found: 258 (M+H). ¹H NMR (400 MHz, DMSO-d₆) δ ppm: 8.53-8.47 (m, 1H), 7.47-7.43 (m, 2H), 4.77 (s, 2H), 0.93 (s, 9H), 0.12 (s, 6H).

### Example 579B methyl (1r,2′S,4S)-2′-[(2R)-3-{[2-({[tert-butyl(dimethyl)silyl]oxy}methyl)pyridin-4-yl]oxy}-2-methylpropyl]-4-(3-chloroanilino)-2′,3′-dihydrospiro[cyclohexane-1,1′-indene]-4-carboxylate

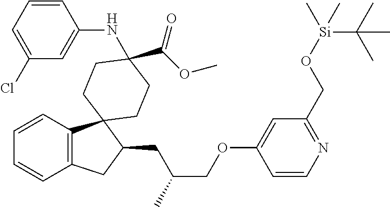 [SMILES: COC(=O)[C@]1(Nc2cccc(Cl)c2)CC[C@@]2(CC1)c1ccccc1C[C@@H]2C[C@@H](C)COc1ccnc(CO[Si](C)(C)C(C)(C)C)c1]

Using General procedure 31a and Preparation 10b1 (100 mg, 0.23 mmol, 1 eq) as the appropriate alcohol and Example 579A (70 mg, 0.27 mmol, 1.2 eq) as the appropriate aryl chloride, Example 579B was obtained as a yellow solid (55 mg, 0.08 mmol, 37%). LRMS calculated for C₃₈H₅₁ClN₂O₄Si: 662; found: 663 (M+H). ¹H NMR (400 MHz, DMSO-d₆) δ ppm: 8.28 (d, J=5.7 Hz, 1H), 7.42-7.37 (m, 1H), 7.22-7.11 (m, 3H), 7.06 (t, J=8.1 Hz, 1H), 6.93 (d, J=2.5 Hz, 1H), 6.83 (dd, J=5.7, 2.6 Hz, 1H), 6.60 (t, J=2.1 Hz, 1H), 6.59-6.55 (m, 1H), 6.48-6.44 (m, 1H), 6.32 (s, 1H), 4.68 (s, 2H), 3.97-3.87 (m, 2H), 3.66 (s, 3H), 2.97 (dd, J=15.6, 7.0 Hz, 1H), 2.49-2.40 (m, 1H), 2.17-1.86 (m, 6H), 1.78-1.68 (m, 1H), 1.53-1.21 (m, 4H), 1.02 (d, J=6.6 Hz, 3H), 0.91 (s, 9H), 0.09 (s, 6H).

### Example 579 (1r,2′S,4S)-4-(3-chloroanilino)-2′-[(2R)-3-{[2-(hydroxymethyl)pyridin-4-yl]oxy}-2-methylpropyl]-2′,3′-dihydrospiro[cyclohexane-1,1′-indene]-4-carboxylic acid

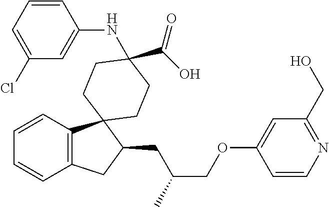 [SMILES: C[C@@H](COc1ccnc(CO)c1)C[C@H]1Cc2ccccc2[C@]12CC[C@@](Nc1cccc(Cl)c1)(C(=O)O)CC2]

Using General procedure 29 and Example 579B (55 mg, 0.08 mmol, 1 eq) as the appropriate silyl derivative, an intermediate was obtained, which was hydrolyzed according to General procedure 33b to obtain Example 579 as a white powder (8.4 mg, 0.02 mmol, 19%). LRMS calculated for C₃₁H₃₅ClN₂O₄: 534; found: 535 (M+H). ¹H NMR (400 MHz, DMSO-d₆) δ ppm: 8.26 (d, J=5.7 Hz, 1H), 7.42-7.36 (m, 1H), 7.24-7.12 (m, 3H), 7.06 (t, J=8.0 Hz, 1H), 6.99 (d, J=2.5 Hz, 1H), 6.80 (dd, J=5.8, 2.6 Hz, 1H), 6.63 (t, J=2.1 Hz, 1H), 6.59-6.52 (m, 2H), 5.35 (t, J=5.9 Hz, 1H), 4.49 (d, J=5.8 Hz, 2H), 3.96-3.87 (m, 2H), 2.99 (dd, J=15.7, 7.1 Hz, 1H), 2.59-2.40 (m, 2H), 2.18-1.82 (m, 6H), 1.79-1.68 (m, 1H), 1.54-1.25 (m, 4H), 1.03 (d, J=6.6 Hz, 3H).

### Example 580

### Example 580A 4-chloro-3-(2,2-dimethylcyclopropyl)pyridine

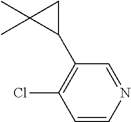

A solution of 3-bromo-4-chloropyridine (0.34 mL, 2.08 mmol, 1 eq), 2-(2,2-dimethylcyclopropyl)-4,4,5,5-tetramethyl-1,3,2-dioxaborolane (0.52 mL, 2.39 mmol, 1.15 eq) and Cs₂CO₃, (1.69 g, 5.2 mmol, 2.5 eq) in toluene (15 mL) and water (1.5 mL) was sparged with N₂. CataCXium© A (37 mg, 0.1 mmol, 0.05 eq) and Pd(OAc)₂ (19 mg, 0.08 mmol, 0.04 eq) were added and the mixture was heated at 100° C. under N₂ for 24 h. The reaction was allowed to cool to rt, sparged with N₂, followed by the addition of further 2-(2,2-dimethylcyclopropyl)-4,4,5,5-tetramethyl-1,3,2-dioxaborolane (0.52 mL, 2.39 mmol, 1.15 eq) and catalytic Pd(dppf)Cl₂×DCM and the mixture was heated at 100° C. under N₂ for 24 h. The mixture was allowed to cool to rt and partitioned between DCM and sat. aq. NaHCO₃ solution.

The aq. phase was extracted with DCM and the combined organic extracts were washed with brine, dried (MgSO₄) and concentrated in vacuo. Purification by automated flash column chromatography (CombiFlash Rf, 12 g RediSep™ silica cartridge) eluting with a gradient of 0-60% EtOAc in heptane afforded racemate Example 580A as a yellow oil (289 mg, 1.59 mmol, 77%). LRMS calculated for C₁₀H₁₂NCl: 181; found: 182 (M+H). ¹H NMR (400 MHz, DMSO-d₆) δ ppm: 8.42-8.37 (m, 2H), 7.54 (d, J=5.2 Hz, 1H), 1.85 (dd, J=8.3, 6.0 Hz, 1H), 1.29 (s, 3H), 1.11 (dd, J=6.0, 5.0 Hz, 1H), 0.89 (dd, J=8.3, 5.0 Hz, 1H), 0.69 (s, 3H).

### Example 580 (1r,2′S,4S)-4-(3-chloroanilino)-2′-[(2R)-3-{[3-(2,2-dimethylcyclopropyl)pyridin-4-yl]oxy}-2-methylpropyl]-2′,3′-dihydrospiro[cyclohexane-1,1′-indene]-4-carboxylic acid

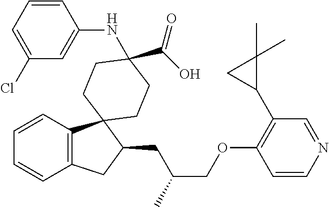 [SMILES: C[C@@H](COc1ccncc1C1CC1(C)C)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and Example 580A (142 mg, 0.78 mmol, 2.3 eq) as the appropriate aryl chloride, a mixture of diastereoisomers, Example 580 was obtained as a cream powder (80.9 mg, 0.14 mmol, 42%). LRMS calculated for C₃₅H₄₁N₂O₃Cl: 572; found: 573 (M+H). ¹H NMR (400 MHz, DMSO-d₆) δ ppm: 8.29-8.25 (m, 1H), 8.06/8.05 (s, 1H), 7.42-7.36 (m, 1H), 7.24-7.11 (m, 3H), 7.08-7.01 (m, 1H), 6.96 (d, J=5.7 Hz, 1H), 6.64-6.60 (m, 1H), 6.57-6.50 (m, 2H), 3.99-3.86 (m, 2H), 3.05-2.96 (m, 1H), 2.61-2.41 (m, 2H), 2.22-1.71 (m, 7H), 1.67-1.30 (m, 5H), 1.15/1.04 (s, 3H), 1.08 (d, J=6.7 Hz, 3H), 0.91-0.84 (m, 1H), 0.72-0.66 (m, 1H), 0.63/0.57 (s, 3H).

### Example 581 (1r,2′S,4S)-4-(3-chloroanilino)-2′-[(2R)-3-{[2-(difluoromethyl)pyridin-4-yl]oxy}-2-methylpropyl]-2′,3′-dihydrospiro[cyclohexane-1,1′-indene]-4-carboxylic acid

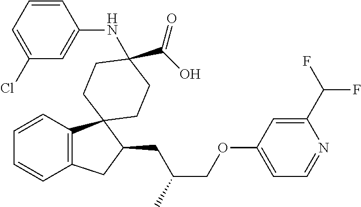 [SMILES: C[C@@H](COc1ccnc(C(F)F)c1)C[C@H]1Cc2ccccc2[C@]12CC[C@@](Nc1cccc(Cl)c1)(C(=O)O)CC2]

Using General procedure 32 and Preparation 10b1 as the appropriate ester and 2-(difluoromethyl)pyridin-4-ol as the appropriate alcohol Example 581 was obtained. ¹H NMR (500 MHz, DMSO-d₆) δ ppm: 12.50 (br s, 1H), 8.46 (d, 1H), 7.38-7.15 (m, 4H), 7.21 (m, 1H), 7.11 (dd, 1H), 7.03 (t, 1H), 6.86 (t, 1H), 6.62 (dd, 1H), 6.54 (dd, 1H), 6.52 (dd, 1H), 6.27 (br s, 1H), 4.01 (m, 2H), 2.97/2.53 (m, 2H), 2.43-1.37 (m, 8H), 2.13 (m, 1H), 1.47 (m, 1H), 1.43/1.32 (m, 2H), 1.03 (d, 3H). HRMS calculated for C₃₁H₃₃N₂O₃F₂Cl: 554.2148; found: 555.2213 (M+H).

### Example 582 (1r,2′S,4S)-4-(3-chloroanilino)-2′-{(2R)-2-methyl-3-[(pyridin-4-yl)oxy]propyl}-2′,3′-dihydrospiro[cyclohexane-1,1′-indene]-4-carboxylic acid

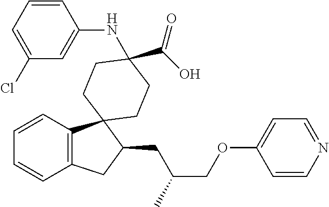 [SMILES: C[C@@H](COc1ccncc1)C[C@H]1Cc2ccccc2[C@]12CC[C@@](Nc1cccc(Cl)c1)(C(=O)O)CC2]

Using General procedure 31c and Preparation 10b1 (140 mg, 0.32 mmol, 1 eq) as the appropriate alcohol and 4-chloropyridine×HCl (109 mg, 0.73 mmol, 2.3 eq) as the appropriate aryl chloride, Example 582 was obtained as a white powder (14 mg, 0.03 mmol, 9%). LRMS calculated for C₃₀H₃₃ClN₂O₃: 504; found: 505 (M+H). ¹H NMR (400 MHz, DMSO-d₆) δ ppm: 8.39-8.34 (m, 2H), 7.42-7.36 (m, 1H), 7.24-7.11 (m, 3H), 7.06 (t, J=8.1 Hz, 1H), 6.97-6.93 (m, 2H), 6.63 (t, J=2.1 Hz, 1H), 6.57-6.52 (m, 2H), 3.97-3.86 (m, 2H), 2.98 (dd, J=15.6, 7.1 Hz, 1H), 2.59-2.38 (m, 2H), 2.20-1.81 (m, 6H), 1.77-1.67 (m, 1H), 1.53-1.27 (m, 4H), 1.02 (d, J=6.6 Hz, 3H).

### Example 583 (1r,2′S,4S)-2′-{(2R)-3-[(2-aminopyridin-4-yl)oxy]-2-methylpropyl}-4-(3-chloroanilino)-2′,3′-dihydrospiro[c clohexane-1,1′-indene]-4-carboxylic acid

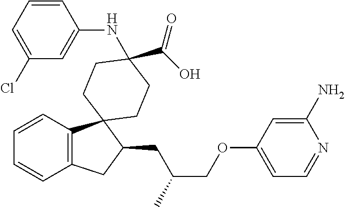 [SMILES: C[C@@H](COc1ccnc(N)c1)C[C@H]1Cc2ccccc2[C@]12CC[C@@](Nc1cccc(Cl)c1)(C(=O)O)CC2]

Using General procedure 31c and Preparation 10b1 as the appropriate ester and 4-chloropyridin-2-amine as the appropriate aryl chloride Example 583 was obtained. ¹H NMR (500 MHz, DMSO-d₆) δ ppm: 12.80 (br s, 1H), 7.68 (d, 1H), 7.38-7.13 (m, 4H), 7.03 (t, 1H), 6.62 (dd, 1H), 6.54 (dd, 1H), 6.52 (dd, 1H), 6.22 (br s, 1H), 6.09 (dd, 1H), 5.93 (d, 1H), 5.75 (s, 2H), 3.77 (m, 2H), 2.97/2.52 (m, 2H), 2.44-1.37 (m, 8H), 2.11 (m, 1H), 1.46 (m, 1H), 1.41/1.28 (m, 2H), 0.99 (d, 3H). HRMS calculated for C₃₀H₃₄N₃O₃Cl: 519.2289; found: 520.2356 (M+H).

### Example 584 (1r,2′S,4S)-4-(3-chloroanilino)-2′-{(2R)-3-[(3,5-dimethylpyridin-4-yl)oxy]-2-methylpropyl}-2′,3′-dihydrospiro[cyclohexane-1,1′-indene]-4-carboxylic acid

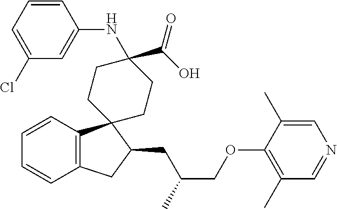 [SMILES: Cc1cncc(C)c1OC[C@H](C)C[C@H]1Cc2ccccc2[C@]12CC[C@@](Nc1cccc(Cl)c1)(C(=O)O)CC2]

Using General procedure 31c and Preparation 10b1 as the appropriate ester and 4-chloro-3,5-dimethyl-pyridine as the appropriate aryl chloride Example 584 was obtained. ¹H NMR (500 MHz, DMSO-d₆) δ ppm: 8.18 (s, 2H), 7.40 (dm, 1H), 7.21 (dm, 1H), 7.17 (tm, 1H), 7.14 (tm, 1H), 7.06 (tm, 1H), 6.62 (t, 1H), 6.54 (dm, 1H), 6.52 (dm, 1H), 3.75/3.73 (t+t, 2H), 2.98/2.55 (dd+dd, 2H), 2.43/2.09/1.97/1.93/1.91/1.71/1.47/1.34 (m, 8H), 2.17 (s, 6H), 2.12 (m, 1H), 1.98 (m, 1H), 1.54/1.42 (m+m, 2H), 1.06 (d, 3H). HRMS calculated for C₃₂H₃₇N₂O₃Cl: 532.2493; found: 533.2565 (M+H).

### Example 585 (1r,2′S,4S)-4-(3-chloroanilino)-2′-[(2R)-2-methyl-3-{[2-(methylamino)pyridin-4-yl]oxy}propyl]-2′,3′-dihydrospiro[cyclohexane-1,1′-indene]-4-carboxylic acid

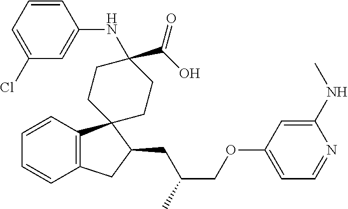 [SMILES: CNc1cc(OC[C@H](C)C[C@H]2Cc3ccccc3[C@]23CC[C@@](Nc2cccc(Cl)c2)(C(=O)O)CC3)ccn1]

Using General procedure 31c and Preparation 10b1 as the appropriate ester and 4-chloro-N-methyl-pyridin-2-amine as the appropriate aryl chloride Example 585 was obtained. ¹H NMR (500 MHz, DMSO-d₆) δ ppm: 7.76 (d, 1H), 7.38 (dm, 1H), 7.21 (dm, 1H), 7.16 (tm, 1H), 7.14 (tm, 1H), 7.05 (t, 1H), 6.62 (t, 1H), 6.54 (m, 2H), 6.14 (dd, 1H), 5.92 (d, 1H), 3.82/3.78 (dd+dd, 2H), 2.97/2.53 (dd+dd, 2H), 2.73 (d, 3H), 2.48-1.32 (m, 8H), 2.11 (m, 1H), 1.96 (m, 1H), 1.41/1.28 (m+m, 2H), 0.99 (d, 3H). HRMS calculated for C₃₁H₃₆N₃O₃Cl: 533.2445; found: 534.2511 (M+H).

### Example 586

### Example 586A (4-chloro-3-methylpyridin-2-yl)methanol

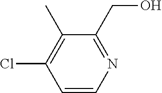

To a solution of methyl 4-chloro-3-methylpyridine-2-carboxylate (225 mg, 1.21 mmol, 1 eq) in MeOH (6 mL), cooled to 0° C. under N₂, was added NaBH₄ (183 mg, 4.85 mmol, 4 eq) portionwise. The reaction was allowed to slowly warm to rt and stirred for 3 h. The mixture was concentrated in vacuo and then partitioned between DCM and water. The organic phase was dried (MgSO₄) and concentrated in vacuo to afford Example 586A as a yellow oil (190 mg, 1.21 mmol, 99%). LRMS calculated for C₇H₈NOCl: 157; found: 158 (M+H). ¹H NMR (400 MHz, DMSO-d₆) δ ppm: 8.30 (d, J=5.3 Hz, 1H), 7.45 (d, J=5.3 Hz, 1H), 5.20 (t, J=5.5 Hz, 1H), 4.62 (d, J=5.5 Hz, 2H), 2.37 (s, 3H).

### Example 586B 4-chloro-3-methyl-2-({[tri(propan-2-yl)silyl]oxy}methyl)pyridine

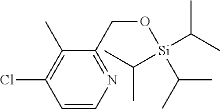 [SMILES: Cc1c(Cl)ccnc1CO[Si](C(C)C)(C(C)C)C(C)C]

To a solution of Example 586A (190 mg, 1.21 mmol, 1 eq) and DIPEA (0.63 mL, 3.62 mmol, 3 eq) in DCM (10 mL), cooled to 0° C., was added triisopropylsilyl trifluoromethanesulfonate (0.49 mL, 1.81 mmol, 1.5 eq) and the mixture was stirred at rt for 48 h. The mixture was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-18% EtOAc in heptane afforded Example 586B as a colorless oil (355 mg, 1.13 mmol, 94%). LRMS calculated for C₁₆H₂₈ClNOSi: 313; found: 314 (M+H). ¹H NMR (400 MHz, DMSO-d₆) δ ppm: 8.30-8.28 (m, 1H), 7.47 (d, J=5.3 Hz, 1H), 4.89 (s, 2H), 2.45 (s, 3H), 1.04-0.98 (m, 21H).

### Example 586C methyl (1r,2′S,4S)-4-(3-chloroanilino)-2′-[(2R)-2-methyl-3-{[3-methyl-2-({[tri(propan-2-yl)silyl]oxy}methyl)pyridin-4-yl]oxy}propyl]-2′,3′-dihydrospiro[cyclohexane-1,1′-indene]-4-carboxylate

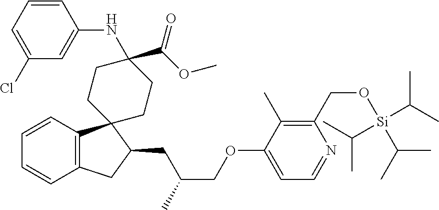 [SMILES: COC(=O)[C@]1(Nc2cccc(Cl)c2)CC[C@@]2(CC1)c1ccccc1C[C@@H]2C[C@@H](C)COc1ccnc(CO[Si](C(C)C)(C(C)C)C(C)C)c1C]

Using General procedure 31a and Preparation 10b1 (150 mg, 0.34 mmol, 1 eq) as the appropriate alcohol and Example 586B (160 mg, 0.51 mmol, 1.5 eq) as the appropriate aryl chloride, Example 586C was obtained as a yellow oil (74 mg, 0.1 mmol, 30%). LRMS calculated for C₄₂H₅₉ClN₂O₄Si: 718; found: 719 (M+H).

### Example 586 (1r,2′S,4S)-4-(3-chloroanilino)-2′-[(2R)-3-{[2-(hydroxymethyl)-3-methylpyridin-4-yl]oxy}-2-methylpropyl]-2′,3′-dihydrospiro[cyclohexane-1,1′-indene]-4-carboxylic acid

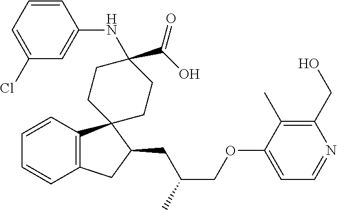 [SMILES: Cc1c(OC[C@H](C)C[C@H]2Cc3ccccc3[C@]23CC[C@@](Nc2cccc(Cl)c2)(C(=O)O)CC3)ccnc1CO]

Using General procedure 29 and Example 586C (74 mg, 0.1 mmol, 1 eq) as the appropriate silyl derivative, the desired intermediate was obtained. Using General procedure 33b, Example 586 was obtained as a white powder (2.9 mg, 0.01 mmol, 8%). LRMS calculated for C₃₂H₃₇ClN₂O₄: 548; found: 549 (M+H). ¹H NMR (400 MHz, DMSO-d₆) δ ppm: 8.22 (d, J=5.5 Hz, 1H), 7.42-7.37 (m, 1H), 7.23-7.11 (m, 3H), 7.09-7.01 (m, 1H), 6.93 (d, J=5.7 Hz, 1H), 6.65-6.61 (m, 1H), 6.58-6.51 (m, 2H), 5.01-4.95 (m, 1H), 4.52 (d, J=4.8 Hz, 2H), 3.97-3.86 (m, 2H), 3.00 (dd, J=15.7, 7.2 Hz, 1H), 2.59-2.40 (m, 2H), 2.18-1.67 (m, 10H), 1.54-1.31 (m, 4H), 1.05 (d, J=6.7 Hz, 3H).

### Example 587 (1r,2′S,4S)-4-(3-chloroanilino)-2′-[(2R)-2-methyl-3-{[3-(propan-2-yl)pyridin-4-yl]oxy}propyl]-2′,3′-dihydrospiro[cyclohexane-1,1′-indene]-4-carboxylic acid

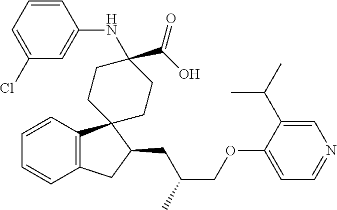 [SMILES: CC(C)c1cnccc1OC[C@H](C)C[C@H]1Cc2ccccc2[C@]12CC[C@@](Nc1cccc(Cl)c1)(C(=O)O)CC2]

Using General procedure 32 and Preparation 10b1 as the appropriate ester and 3-isopropylpyridin-4-ol as the appropriate alcohol Example 587 was obtained. ¹H NMR (500 MHz, DMSO-d₆) δ ppm: 12.64 (br s, 1H), 8.26 (d, 1H), 8.23 (s, 1H), 7.39-7.11 (m, 4H), 7.05 (t, 1H), 6.94 (d, 1H), 6.62 (t, 1H), 6.54 (dm, 1H), 6.53 (dm, 1H), 6.22 (br s, 1H), 3.92/3.88 (dd+dd, 2H), 3.08 (sp, 1H), 3.01/2.54 (dd+dd, 2H), 2.50-1.33 (m, 8H), 2.16 (m, 1H), 2.02 (m, 1H), 1.46/1.36 (m+m, 2H), 1.17 (d, 3H), 1.15 (d, 3H), 1.05 (d, 3H). HRMS calculated for C₃₃H₃₉N₂O₃Cl: 546.2649; found: 547.2725 (M+H).

### Example 588 (1r,2′S,4S)-4-(3-chloroanilino)-2′-{(2R)-3-[(3-ethylpyridin-4-yl)oxy]-2-methylpropyl}-2′,3′-dihydrospiro[cyclohexane-1,1′-indene]-4-carboxylic acid

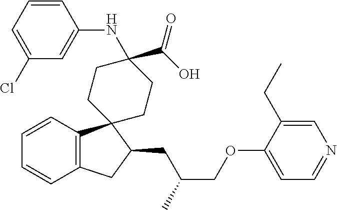 [SMILES: CCc1cnccc1OC[C@H](C)C[C@H]1Cc2ccccc2[C@]12CC[C@@](Nc1cccc(Cl)c1)(C(=O)O)CC2]

Using General procedure 32 and Preparation 10b1 as the appropriate ester and 3-ethylpyridin-4-ol as the appropriate alcohol Example 588 was obtained. ¹H NMR (500 MHz, DMSO-d₆) δ ppm: 12.69 (br s, 1H), 8.26 (d, 1H), 8.19 (s, 1H), 7.41-7.11 (m, 4H), 7.04 (t, 1H), 6.94 (d, 1H), 6.62 (t, 1H), 6.53 (dm, 2H), 6.24 (br s, 1H), 3.91 (d, 2H), 3.00/2.54 (dd+dd, 2H), 2.51 (q, 2H), 2.50-1.31 (m, 8H), 2.13 (m, 1H), 2.00 (m, 1H), 1.45/1.36 (m+m, 2H), 1.08 (t, 3H), 1.04 (d, 3H). HRMS calculated for C₃₂H₃₇N₂O₃Cl: 532.2493; found: 533.2571 (M+H).

### Example 589 (1r,4R)-4-(3-chloroanilino)-2′-{(2R)-2-methyl-3-[(thieno[3,2-b]pyridin-7-yl)oxy]propyl}spiro[cyclohexane-1,1′-indene]-4-carboxylic acid

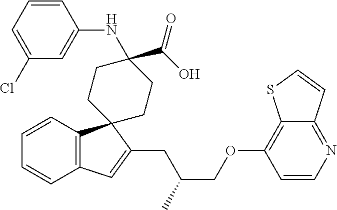 [SMILES: C[C@@H](COc1ccnc2ccsc12)CC1=Cc2ccccc2[C@]12CC[C@@](Nc1cccc(Cl)c1)(C(=O)O)CC2]

Using General Procedure 30b and Preparation 10a as the appropriate indene and thieno[3,2-b]pyridin-7-ol as the appropriate alcohol, afforded an intermediate which was hydrolyzed according to General procedure 33b to afford Example 589 as a white powder. LRMS calculated for C₃₂H₃₁ClN₂O₃S: 558; found: 559 (M+H). ¹HNMR (400 MHz, DMSO-d₆) δ ppm: 12.80 (br s, 1H), 8.51 (d, J=5.5 Hz, 1H), 7.93 (d, J=5.4 Hz, 1H), 7.70-7.63 (m, 1H), 7.50 (d, J=5.4 Hz, 1H), 7.32-7.27 (m, 1H), 7.26-7.20 (m, 1H), 7.15-7.07 (m, 2H), 6.99 (d, J=5.5 Hz, 1H), 6.65 (t, J=2.1 Hz, 1H), 6.62-6.55 (m, 3H), 6.37 (br s, 1H), 4.28 (dd, J=9.5, 4.5 Hz, 1H), 4.18 (dd, J=9.5, 5.6 Hz, 1H), 2.58-2.46 (m, 2H), 2.46-2.32 (m, 2H), 2.29-2.08 (m, 5H), 1.13 (d, J=6.3 Hz, 3H), 0.96-0.81 (m, 2H).

### Example 590 (1r,2′R,4R)-4-(3-chloroanilino)-2′-{(2R)-2-methyl-3-[(thieno[3,2-b]pyridin-7-yl)oxy]propyl}-2′,3′-dihydrospiro[cyclohexane-1,1′-indene]-4-carboxylic acid

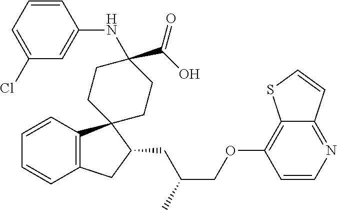 [SMILES: C[C@@H](COc1ccnc2ccsc12)C[C@@H]1Cc2ccccc2[C@]12CC[C@@](Nc1cccc(Cl)c1)(C(=O)O)CC2]

and

### Example 591 (1r,2′S,4S)-4-(3-chloroanilino)-2′-{(2R)-2-methyl-3-[(thieno[3,2-b]pyridin-7-yl)oxy]propyl}-2′,3′-dihydrospiro[cyclohexane-1,1′-indene]-4-carboxylic acid

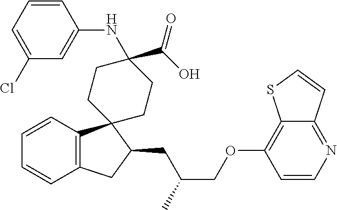 [SMILES: C[C@@H](COc1ccnc2ccsc12)C[C@H]1Cc2ccccc2[C@]12CC[C@@](Nc1cccc(Cl)c1)(C(=O)O)CC2]

Using General Procedure 30b and Preparation 10b as the appropriate indane and thieno[3,2-b]pyridin-7-ol as the appropriate alcohol, afforded an intermediate which was hydrolyzed according to General procedure 33b to afford a mixture of diastereoisomers. They were separated by chiral chromatography. Column: AD, 50×500 mm, 20 μm. Eluents: iPrOH+0.05% HCOOH. The diastereoisomer eluting earlier was collected as Example 590. HRMS calculated for C₃₂H₃₃N₂O₃SCl: 560.1901; found: 561.1979 (M+H). ¹H NMR (400 MHz, DMSO-d₆) δ ppm: 8.51 (d, J=5.5 Hz, 1H), 7.92 (d, J=5.4 Hz, 1H), 7.49 (d, J=5.4 Hz, 1H), 7.43-7.37 (m, 1H), 7.24-7.11 (m, 3H), 7.06-6.98 (m, 2H), 6.61 (t, J=2.1 Hz, 1H), 6.56-6.49 (m, 2H), 6.09 (br s, 1H), 4.25 (dd, J=9.7, 5.1 Hz, 1H), 4.14 (dd, J=9.7, 6.1 Hz, 1H), 3.06 (dd, J=15.7, 7.1 Hz, 1H), 2.61 (dd, J=15.7, 8.4 Hz, 1H), 2.47-2.36 (m, 1H), 2.24-1.66 (m, 8H), 1.47-1.22 (m, 3H), 1.11 (d, J=6.8 Hz, 3H).

The diastereoisomer eluting later was collected as Example 591. HRMS calculated for C₃₂H₃₃N₂O₃SCl: 560.1901; found: 561.1966 (M+H). ¹H NMR (400 MHz, DMSO-d₆) δ ppm: 8.52 (d, J=5.5 Hz, 1H), 8.02 (d, J=5.4 Hz, 1H), 7.51 (d, J=5.4 Hz, 1H), 7.42-7.36 (m, 1H), 7.24-7.11 (m, 3H), 7.08-6.98 (m, 2H), 6.64 (t, J=2.1 Hz, 1H), 6.58-6.51 (m, 2H), 4.21-4.08 (m, 2H), 3.02 (dd, J=15.6, 7.1 Hz, 1H), 2.62-2.39 (m, 2H), 2.21-1.67 (m, 7H), 1.55-1.33 (m, 4H), 1.08 (d, J=6.6 Hz, 3H).

### Example 592

### Example 592A [(2R)-3-bromo-2-methylpropoxy](tert-butyl)dimethylsilane

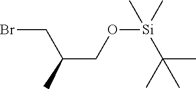

Using General procedure 41b and TBDMS-Cl as the appropriate silyl chloride and (2R)-3-bromo-2-methylpropan-1-ol as the appropriate alcohol, Example 592A was obtained as a colourless oil. ¹H NMR (400 MHz, DMSO-d₆) δ ppm: 3.58-3.45 (m, 4H), 2.00-1.86 (m, 1H), 0.93 (d, J=6.8 Hz, 3H), 0.88 (s, 9H), 0.05 (s, 6H).

### Example 592B bromido[(2R)-3-{[tert-butyl(dimethyl)silyl]oxy}-2-methylpropyl]zinc

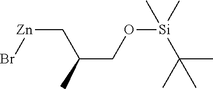

Using General procedure 4 and Example 592A as the appropriate bromo compound, Example 592B was obtained.

### Example 592C methyl (1r,4S)-4-(3-chloroanilino)-2′-[(2S)-3-hydroxy-2-methylpropyl]spiro[cyclohexane-1,1′-indene]-4-carboxylate

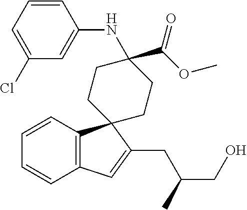 [SMILES: COC(=O)[C@]1(Nc2cccc(Cl)c2)CC[C@]2(CC1)C(C[C@H](C)CO)=Cc1ccccc12]

Using General Procedure 27a and Preparation 6c as the appropriate 2-bromo-indene derivative and Example 592B as the appropriate Zn reagent, afforded an intermediate that was treated according to General Procedure 29 to give Example 592C as a beige powder. LRMS calculated for C₂₆H₃₀ClNO₃: 439; found: 440 (M+H).

### Example 592D methyl (1r,4S)-4-(3-chloroanilino)-2′-[(2S)-3-hydroxy-2-methylpropyl]-2′,3′-dihydrospiro[cyclohexane-1,1′-indene]-4-carboxylate

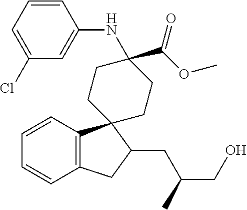

Using General Procedure 19 and Example 592C as the appropriate indene, a racemate, Example 592D was isolated as a colourless gum. LRMS calculated for C₂₆H₃₂ClNO₃: 441; found: 442 (M+H).

### Example 592 (1r,4S)-4-(3-chloroanilino)-2′-{(2S)-2-methyl-3-[(thieno[3,2-b]pyridin-7-yl)oxy]propyl}-2′,3′-dihydrospiro[cyclohexane-1,1′-indene]-4-carboxylic acid

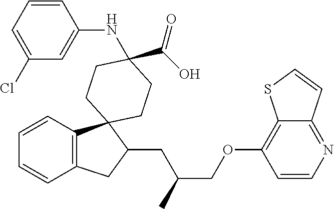

Using General Procedure 30b and Example 592D as the appropriate indane and thieno[3,2-b]pyridin-7-ol as the appropriate alcohol, afforded an intermediate which was treated according to General procedure 33b to obtain a mixture of diastereoisomers, Example 592, isolated as a white powder. LRMS calculated for C₃₂H₃₃ClN₂O₃S: 560; found: 561 (M+H). ¹H NMR (400 MHz, DMSO-d₆) δ ppm: 12.67 (br s, 1H), 8.54-8.49 (m, 1H), 8.02/7.91 (d, J=5.4 Hz, 1H), 7.51/7.49 (d, J=5.4 Hz, 1H), 7.43-7.36 (m, 1H), 7.24-7.11 (m, 3H), 7.08-6.99 (m, 2H), 6.63/6.60 (t, J=2.1 Hz, 1H), 6.58-6.49 (m, 2H), 6.16 (br s, 1H), 4.29-4.08 (m, 2H), 3.10-2.97 (m, 1H), 2.67-2.36 (m, 2H), 2.25-1.21 (m, 11H), 1.15-1.04 (m, 3H).

### Example 593 (1r,4R)-4-(3-chloroanilino)-2′-{(2R)-3-[(6,7-dihydro-5H-cyclopenta[d]pyrimidin-4-yl)oxy]-2-methylpropyl}-2′,3′-dihydrospiro[cyclohexane-1,1′-indene]-4-carboxylic acid

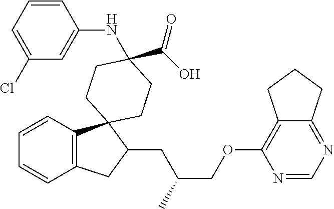

Using General Procedure 31b and Preparation 10b as the appropriate alcohol and 4-chloro-6,7-dihydro-5H-cyclopenta[d]pyrimidine as the appropriate aryl chloride, a mixture of diastereoisomers, Example 593 was isolated as a beige powder. LRMS calculated for C₃₂H₃₆ClN₃O₃: 545; found: 546 (M+H). ¹H NMR (400 MHz, DMSO-d₆) δ ppm: 8.54/8.52 (s, 1H), 7.43-7.34 (m, 1H), 7.24-7.10 (m, 3H), 7.09-7.01 (m, 1H), 6.64-6.59 (m, 1H), 6.58-6.49 (m, 2H), 6.21 (br s, 1H), 4.36-4.18 (m, 2H), 3.07-2.92 (m, 1H), 2.92-2.64 (m, 4H), 2.64-2.32 (m, 2H), 2.17-1.13 (m, 13H), 1.07-0.97 (m, 3H).

### Example 594 (1r,4R)-4-(3-chloroanilino)-2′-{(2R)-3-[(1H-indol-4-yl)oxy]-2-methylpropyl}-2′,3′-dihydrospiro[cyclohexane-1,1′-indene]-4-carboxylic acid

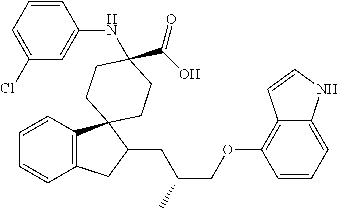

Using General Procedure 30b and Preparation 10b as the appropriate indane and 4-hydroxyindole as the appropriate alcohol, an intermediate was obtained, which was hydrolyzed according to General Procedure 33b to obtain a mixture of diastereoisomers, Example 594 as a white powder. LRMS calculated for C₃₃H₃₅ClN₂O₃: 542; found: 543 (M+H). ¹H NMR (400 MHz, DMSO-d₆) δ ppm: 11.08-10.99 (m, 1H), 7.43-7.35 (m, 1H), 7.25-7.09 (m, 4H), 7.08-7.01 (m, 1H), 7.00-6.92 (m, 2H), 6.65-6.44 (m, 4H), 6.41-6.36 (m, 1H), 6.10 (br s, 1H), 4.10-3.85 (m, 2H), 3.11-2.97 (m, 1H), 2.67-2.35 (m, 2H), 2.27-1.21 (m, 11H), 1.17-1.04 (m, 3H).

### Example 595 (1r,4R)-4-(3-chloroanilino)-2′-{(2R)-3-[(5,5-dimethyl-6,7-dihydro-5H-cyclopenta[d]pyrimidin-4-yl)oxy]-2-methylpropyl}-2′,3′-dihydrospiro[cyclohexane-1,1′-indene]-4-carboxylic acid

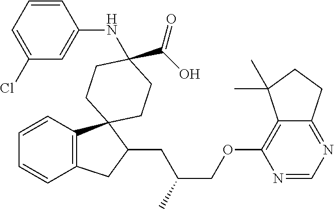

Using General Procedure 31b and Preparation 10b as the appropriate alcohol and Example 466E as the appropriate aryl chloride, a mixture of diastereoisomers, Example 595 was isolated as a cream powder. LRMS calculated for C₃₄H₄₀ClN₃O₃: 573; found: 574 (M+H). ¹H NMR (400 MHz, DMSO-d₆) δ ppm: 12.67 (br s, 1H), 8.53/8.52 (s, 1H), 7.44-7.33 (m, 1H), 7.25-7.09 (m, 3H), 7.09-7.01 (m, 1H), 6.65-6.45 (m, 3H), 6.15 (br s, 1H), 4.38-4.17 (m, 2H), 3.08-2.95 (m, 1H), 2.91-2.76 (m, 2H), 2.64-2.35 (m, 2H), 2.20-1.17 (m, 19H), 1.11-1.01 (m, 3H).

### Example 596 (1r,4R)-4-anilino-2′-{(2R)-3-[(6,7-dihydro-5H-cyclopenta[b]pyridin-4-yl)oxy]-2-methylpropyl}-2′,3′-dihydrospiro[cyclohexane-1,1′-indene]-4-carboxylic acid

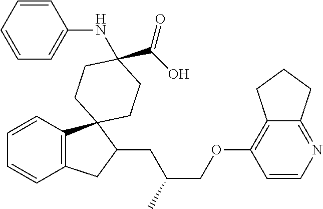

At the isolation of Example 598 and Example 599, Example 596 was also obtained as a mixture of diastereoisomers, isolated as a white powder. LRMS calculated for C₃₃H₃₈N₂O₃: 510; found: 511 (M+H). ¹H NMR (400 MHz, DMSO-d₆) δ ppm: 8.19-8.13 (m, 1H), 7.42-7.35 (m, 1H), 7.23-7.11 (m, 3H), 7.08-7.00 (m, 2H), 6.81-6.76 (m, 1H), 6.64-6.50 (m, 3H), 4.05-3.87 (m, 2H), 3.06-2.93 (m, 1H), 2.90-2.68 (m, 4H), 2.63-2.35 (m, 2H), 2.18-1.16 (m, 13H), 1.10-0.98 (m, 3H).

### Example 597 (1r,4R)-4-(3-chloroanilino)-2′-[(2R)-2-methyl-3-{[(5R)-5-methyl-6,7-dihydro-5H-cyclopenta[d]pyrimidin-4-yl]oxy}propyl]-2′,3′-dihydrospiro[cyclohexane-1,1′-indene]-4-carboxylic acid

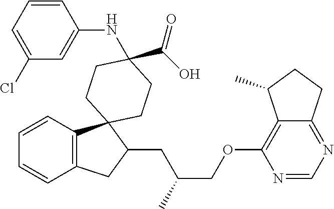

Using General Procedure 31b and Preparation 10b as the appropriate alcohol and Example 472C as the appropriate aryl chloride, a mixture of diastereoisomers, Example 597 was isolated as a yellow powder. LRMS calculated for C₃₃H₃₈ClN₃O₃: 559; found: 560 (M+H). ¹H NMR (400 MHz, DMSO-d₆) δ ppm: 12.67 (br s, 1H), 8.54-8.51 (m, 1H), 7.41-7.33 (m, 1H), 7.24-7.10 (m, 3H), 7.09-7.02 (m, 1H), 6.64-6.59 (m, 1H), 6.58-6.48 (m, 2H), 6.19 (br s, 1H), 4.40-4.12 (m, 2H), 3.29-1.12 (m, 22H), 1.06/1.01 (d, J=6.7 Hz, 3H).

### Example 598 (1r,2′R,4R)-4-(3-chloroanilino)-2′-{(2R)-3-[(6,7-dihydro-5H-cyclopenta[b]pyridin-4-yl)oxy]-2-methylpropyl}-2′,3′-dihydrospiro[cyclohexane-1,1′-indene]-4-carboxylic acid

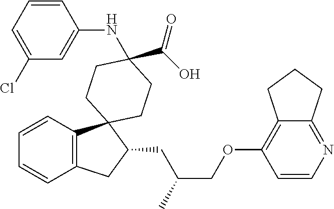 [SMILES: C[C@@H](COc1ccnc2c1CCC2)C[C@@H]1Cc2ccccc2[C@]12CC[C@@](Nc1cccc(Cl)c1)(C(=O)O)CC2]

### Example 599 (1r,2′S,4S)-4-(3-chloroanilino)-2′-{(2R)-3-[(6,7-dihydro-5H-cyclopenta[b]pyridin-4-yl)oxy]-2-methylpropyl}-2′,3′-dihydrospiro[cyclohexane-1,1′-indene]-4-carboxylic acid

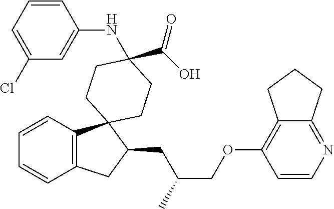 [SMILES: C[C@@H](COc1ccnc2c1CCC2)C[C@H]1Cc2ccccc2[C@]12CC[C@@](Nc1cccc(Cl)c1)(C(=O)O)CC2]

Using General Procedure 31a and Preparation 10b as the appropriate alcohol and 4-chloro-6,7-dihydro-5H-cyclopenta[b]pyridine as the appropriate aryl chloride, an intermediate was obtained, which was hydrolyzed according to General Procedure 33b to obtain a mixture of diastereoisomers. They were separated by chiral chromatography. Column: AD, 50×500 mm, 20 μm. Eluents: 50:50 nPrOH/Heptane+0.05% HCOOH. The diastereoisomer eluting earlier was collected and further purified via prep RP-HPLC using 25 mM aq. NH₄HCO₃ solution and MeCN as eluents to obtain Example 598. ¹H NMR (500 MHz, DMSO-d₆) δ ppm: 8.14 (d, 1H), 7.38 (m, 1H), 7.20 (m, 1H), 7.14 (m, 2H), 7.04 (t, 1H), 6.77 (d, 1H), 6.61 (t, 1H), 6.55 (dd, 1H), 6.51 (dd, 1H), 6.18 (br s, 1H), 3.99/3.93 (dd+dd, 2H), 3.00/2.58 (dd+dd, 2H), 2.80 (m, 2H), 2.69 (t, 2H), 2.45-1.28 (m, 8H), 2.11 (m, 1H), 2.03 (m, 1H), 1.88 (m, 2H), 1.67/1.20 (m+m, 2H), 1.05 (d, 3H). HRMS calculated for C₃₃H₃₇N₂O₃Cl: 544.2493; found: 545.2563 (M+H).

The diastereoisomer eluting later was collected and further purified via prep RP-HPLC using 25 mM aq. NH₄HCO₃ solution and MeCN as eluents to obtain Example 599. ¹H NMR (500 MHz, DMSO-d₆) δ ppm: 8.15 (d, 1H), 7.38 (d, 1H), 7.19 (m, 1H), 7.15 (m, 2H), 7.04 (t, 1H), 6.77 (d, 1H), 6.62 (t, 1H), 6.54 (dm, 1H), 6.54 (dm, 1H), 6.25 (br s, 1H), 3.91 (m, 2H), 2.97/2.51 (dd+dd, 2H), 2.84 (t, 2H), 2.74 (m, 2H), 2.48-1.27 (m, 8H), 2.10 (m, 1H), 1.99 (m, 2H), 1.98 (m, 1H), 1.42/1.33 (m+m, 2H), 1.01 (d, 3H). HRMS calculated for C₃₃H₃₇N₂O₃Cl: 544.2493; found: 545.2566 (M+H).

### Example 600 (1r,4R)-4-(3-chloroanilino)-2′-{(2R)-2-methyl-3-[(5-methyl-6,7-dihydro-5H-cyclopenta[b]pyridin-4-yl)oxy]propyl}-2′,3′-dihydrospiro[cyclohexane-1,1′-indene]-4-carboxylic acid, diastereoisomer 1

and

### Example 601 (1r,4R)-4-(3-chloroanilino)-2′-{(2R)-2-methyl-3-[(5-methyl-6,7-dihydro-5H-cyclopenta[b]pyridin-4-yl)oxy]propyl}-2′,3′-dihydrospiro[cyclohexane-1,1′-indene]-4-carboxylic acid, diastereoisomer 2

### Example 602 (1r,4R)-4-(3-chloroanilino)-2′-{(2R)-2-methyl-3-[(5-methyl-6,7-dihydro-5H-cyclopenta[b]pyridin-4-yl)oxy]propyl}-2′,3′-dihydrospiro[cyclohexane-1,1′-indene]-4-carboxylic acid, diastereoisomer 3 and diastereoisomer 4

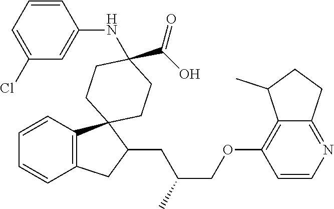

Using General Procedure 31c and Preparation 10b as the appropriate alcohol and Example 477D as the appropriate aryl chloride, a mixture of 4 diastereoisomers was obtained. They were separated by chiral chromatography. Column: AD, 50×500 mm, 20 μm. Eluents: 30:70 EtOH/Heptane+0.05% HCOOH. The diastereoisomer eluting firstly was collected as Example 600. HRMS calculated for C₃₄H₃₉N₂O₃Cl: 558.2649; found: 559.2717 (M+H).

The mixture of 3 diastereoisomers eluting secondly was collected and purified again by chiral chromatography. Column: AD, 50×500 mm, 20 μm. Eluents: 25:75 iPrOH/Heptane+0.05% TFA. The diastereoisomer eluting firstly was collected as Example 601. HRMS calculated for C₃₄H₃₉N₂O₃Cl: 558.2649; found: 559.2715 (M+H).

The diastereoisomer mixture eluting secondly was collected as Example 602. HRMS calculated for C₃₄H₃₉N₂O₃Cl: 558.2649; found: 559.2721 (M+H).

### Example 603 (1r,2′S,4S)-4-(3-chloroanilino)-2′-{(2R)-3-[(7-fluoro-6,7-dihydro-5H-cyclopenta[b]pyridin-4-yl)oxy]-2-methylpropyl}-2′,3′-dihydrospiro[cyclohexane-1,1′-indene]-4-carboxylic acid

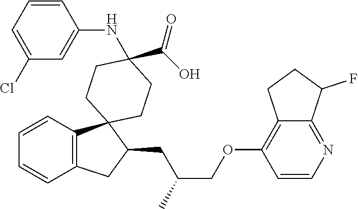 [SMILES: C[C@@H](COc1ccnc2c1CCC2F)C[C@H]1Cc2ccccc2[C@]12CC[C@@](Nc1cccc(Cl)c1)(C(=O)O)CC2]

Using General Procedure 31a and Preparation 10b1 as the appropriate alcohol and Example 476A as the appropriate aryl chloride, an intermediate was obtained, which was hydrolyzed as described in General Procedure 33b to obtain Example 603 as a mixture of diastereoisomers, isolated as a white powder. LRMS calculated for C₃₃H₃₆ClFN₂O₃: 562; found: 563 (M+H). ¹H NMR (400 MHz, DMSO-d₆) δ ppm: 8.37 (d, J=5.6 Hz, 1H), 7.42-7.36 (m, 1H), 7.24-7.11 (m, 3H), 7.08-6.98 (m, 2H), 6.64-6.61 (m, 1H), 6.57-6.52 (m, 2H), 5.98-5.78 (m, 1H), 4.05-3.92 (m, 2H), 3.04-2.83 (m, 2H), 2.81-2.68 (m, 1H), 2.57-2.32 (m, 3H), 2.24-1.82 (m, 7H), 1.79-1.68 (m, 1H), 1.54-1.31 (m, 4H), 1.04 (d, J=6.6 Hz, 3H).

### Example 604 (1r,2′S,4S)-4-(3-chloroanilino)-2′-{(2R)-2-methyl-3-[(thieno[2,3-b]pyridin-4-yl)oxy]propyl}-2′,3′-dihydrospiro[cyclohexane-1,1′-indene]-4-carboxylic acid

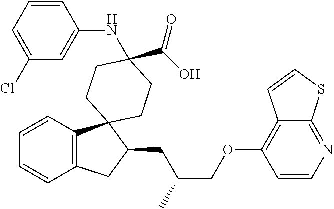 [SMILES: C[C@@H](COc1ccnc2sccc12)C[C@H]1Cc2ccccc2[C@]12CC[C@@](Nc1cccc(Cl)c1)(C(=O)O)CC2]

Using General procedure 32 and Preparation 10b1 as the appropriate indane and thieno[2,3-b]pyridin-4-ol as the appropriate alcohol Example 604 was obtained. ¹H NMR (500 MHz, DMSO-d₆) δ ppm: 8.40 (d, 1H), 7.68 (d, 1H), 7.42-7.10 (m, 4H), 7.37 (d, 1H), 7.02 (t, 1H), 7.00 (d, 1H), 6.61 (t, 1H), 6.55 (dd, 1H), 6.52 (dd, 1H), 6.25 (s, 1H), 4.11/4.05 (dd+dd, 2H), 3.00/2.57 (d+d, 2H), 2.49-1.32 (m, 8H), 2.14 (m, 1H), 2.08 (m, 1H), 1.48/1.41 (m+m, 2H), 1.08 (d+d, 3H). HRMS calculated for C₃₂H₃₃N₂O₃SCl: 560.1901; found: 561.1977 (M+H).

### Example 605 (1r,2′S,4S)-4-(3-chloroanilino)-2′-{(2R)-2-methyl-3-[(3-methylthieno[2,3-b]pyridin-4-yl)oxy]propyl}-2′,3′-dihydrospiro[cyclohexane-1,1′-indene]-4-carboxylic acid

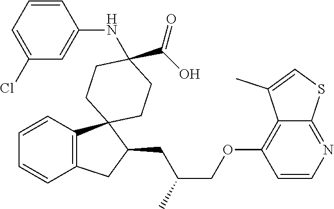 [SMILES: Cc1csc2nccc(OC[C@H](C)C[C@H]3Cc4ccccc4[C@]34CC[C@@](Nc3cccc(Cl)c3)(C(=O)O)CC4)c12]

Using General procedure 32 and Preparation 10b1 as the appropriate indane and 3-methylthieno[2,3-b]pyridin-4-ol as the appropriate alcohol Example 605 was obtained. ¹H NMR (500 MHz, DMSO-d₆) δ ppm: 8.34 (d, 1H), 7.38 (d, 1H), 7.22 (s, 1H), 7.21-7.11 (m, 3H), 7.03 (t, 1H), 6.94 (d, 1H), 6.62 (t, 1H), 6.53 (m, 1H), 6.53 (m, 1H), 4.05/4.03 (dd+dd, 2H), 3.02/2.56 (dd+dd, 2H), 2.52-1.34 (m, 8H), 2.46 (s, 3H), 2.17 (m, 1H), 2.08 (m, 1H), 1.53/1.40 (m+m, 2H), 1.09 (d, 3H). HRMS calculated for C₃₃H₃₅N₂O₃SCl: 574.2057; found: 575.2115 (M+H).

### Example 606 (1r,2′S,4S)-4-(3-chloroanilino)-2′-{(2R)-3-[(furo[2,3-b]pyridin-4-yl)oxy]-2-methylpropyl}-2′,3′-dihydrospiro[cyclohexane-1,1′-indene]-4-carboxylic acid

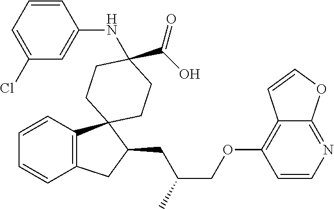 [SMILES: C[C@@H](COc1ccnc2occc12)C[C@H]1Cc2ccccc2[C@]12CC[C@@](Nc1cccc(Cl)c1)(C(=O)O)CC2]

Using General procedure 31c and Preparation 10b1 as the appropriate indane and 4-chlorofuro[2,3-b]pyridine as the appropriate aryl chloride Example 606 was obtained. ¹H NMR (500 MHz, DMSO-d₆) δ ppm: 12.50 (br s, 1H), 8.16 (d, 1H), 7.93 (d, 1H), 7.42-7.11 (m, 4H), 7.02 (t, 1H), 6.95 (m, 2H), 6.62 (t, 1H), 6.54 (dd, 1H), 6.52 (dd, 1H), 6.22 (br s, 1H), 4.12/4.07 (m+m, 2H), 2.99/2.56 (dd+dd, 2H), 2.48-1.33 (m, 8H), 2.14 (m, 1H), 2.06 (m, 1H), 1.47/1.39 (m+m, 2H), 1.07 (d, 3H). HRMS calculated for C₃₂H₃₃N₂O₄Cl: 544.2129; found: 545.2200 (M+H).

### Example 607

### Example 607A methyl (1r,4R)-4-(3-chloroanilino)-2′-[(2R)-2-methyl-3-oxopropyl]-2′,3′-dihydrospiro[cyclohexane-1,1′-indene]-4-carboxylate

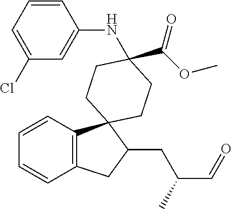

To a solution of Preparation 10b (290 mg, 0.66 mmol, 1 eq) in DCM (10 mL) at 0° C. was added DMP (334 mg, 0.79 mmol, 1.2 eq) and the mixture was stirred at rt for 2.5 h. The mixture was partitioned between EtOAc and water, and the organic phase was washed with a 1:1 solution of aq. Na₂S₂O₃ solution and sat. aq. NaHCO₃ solution, then brine, then dried (MgSO₄) and concentrated in vacuo to afford Example 607A (288 mg, 0.66 mmol, 100%). LRMS calculated for C₂₆H₃₀ClNO₃: 439; found: 440 (M+H).

### Example 607B methyl (1r,4r)-4-(3-chloroanilino)-2′-(2-fluoro-3-hydroxy-2-methylpropyl)-2′,3′-dihydrospiro[cyclohexane-1,1′-indene]-4-carboxylate

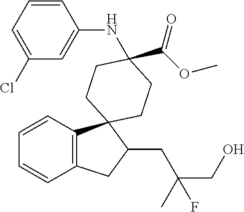

To a solution of Example 607A (288 mg, 0.66 mmol, 1 eq) in THE (2.5 mL) and TFA (9.82 μL, 0.13 mmol, 0.2 eq) was added methyl (R)-(+)-alpha,alpha-diphenyl-2-pyrrolidinemethanol trimethylsilyl ether (41 μL, 0.13 mmol, 0.2 eq) and NFSI (229 mg, 0.73 mmol, 1.1 eq). The mixture was stirred at rt for 48 h. Then MeOH (4 mL) and NaBH₄ (75 mg, 1.98 mmol, 3 eq) were added and the mixture was stirred for a further 4 h at rt. The mixture was partitioned between DCM and water, and the organic phase was washed with sat. aq. NaHCO₃ solution, brine, dried (PTFE phase separator) and concentrated in vacuo. Purification by automated flash chromatography (CombiFlash Rf, 12 g RediSep™ silica cartridge) eluting with a gradient of 0-40% EtOAc in heptane afforded a mixture of diastereoisomers, Example 607B, isolated as a yellow glass (75 mg, 0.16 mmol, 25%). LRMS calculated for C₂₆H₃₁ClFNO₃: 459; found: 460 (M+H).

### Example 607 (1r,4r)-4-(3-chloroanilino)-2′-{3-[(6,7-dihydro-5H-cyclopenta[b]pyridin-4-yl)oxy]-2-fluoro-2-methylpropyl}-2′,3′-dihydrospiro[cyclohexane-1,1′-indene]-4-carboxylic acid

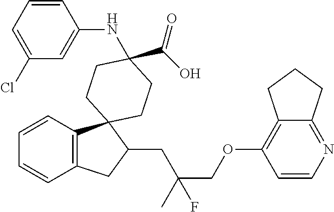

Using General Procedure 31c and Example 607B as the appropriate alcohol and 4-chloro-6,7-dihydro-5H-cyclopenta[b]pyridine as the appropriate aryl chloride, a mixture of 4 diastereoisomers, Example 607, was isolated as a white powder. LRMS calculated for C₃₃H₃₆ClFN₂O₃: 562; found: 563 (M+H). ¹H NMR (400 MHz, DMSO-d₆) δ ppm: 8.20-8.15 (m, 1H), 7.52-7.44 (m, 1H), 7.25-7.11 (m, 3H), 7.11-7.01 (m, 1H), 6.87-6.81 (m, 1H), 6.65-6.61 (m, 1H), 6.59-6.50 (m, 2H), 6.22 (br s, 1H), 4.27-4.10 (m, 2H), 3.08-2.97 (m, 1H), 2.88-2.79 (m, 2H), 2.79-2.59 (m, 3H), 2.59-2.40 (m, 1H), 2.15-1.70 (m, 10H), 1.54-1.38 (m, 4H), 1.27-1.18 (m, 1H).

### Example 608 and Example 609

### Example 608A 7-{[tert-butyl(dimethyl)silyl]oxy}-4-chloro-6,7-dihydro-5H-cyclopenta[b]pyridine, enantiomer 1

and

### Example 609A 7-{[tert-butyl(dimethyl)silyl]oxy}-4-chloro-6,7-dihydro-5H-cyclopenta[b]pyridine, enantiomer 2

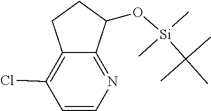 [SMILES: CC(C)(C)[Si](C)(C)OC1CCc2c(Cl)ccnc21]

To a solution of 4-chloro-6,7-dihydro-5H-cyclopenta[b]pyridin-7-ol (465 mg, 2.74 mmol, 1 eq) and DIPEA (1.36 mL, 8.22 mmol, 3 eq) in DCM (10 mL), cooled to 0° C., was added TBDMS-OTf (1.58 mL, 6.85 mmol, 2.5 eq) and the mixture was stirred at 0° C. for 1 h, then at rt for 1 h.

The mixture was partitioned between DCM and water, and the organic phase was washed with brine, dried (PTFE phase separator) and concentrated in vacuo. Purification by automated flash chromatography (CombiFlash Rf, 24 g RediSep™ silica cartridge) eluting with a gradient of 0-17% EtOAc in heptane afforded a racemate as a colourless oil (760 mg, 2.68 mmol, 98%). The enantiomers were separated by chiral chromatography. Column: IC, 50×500 mm, 20 μm. Eluent: Heptane. The enantiomer eluting earlier was collected as Example 609A. ¹H NMR (500 MHz, DMSO-d₆) δ ppm: 8.40 (d, 1H), 7.38 (d, 1H), 5.20 (dd, 1H), 2.98/2.79 (m+m, 2H), 2.44/1.88 (m+m, 2H), 0.89 (s, 9H), 0.16/0.11 (s+s, 3H). HRMS calculated for C₁₄H₂₂ClNOSi: 283.1159; found: 284.1230 (M+H).

The enantiomer eluting second was collected as Example 608A. HRMS calculated for C₁₄H₂₂ClNOSi: 283.1159; found: 284.1227 (M+H).

### Example 608B methyl (1r,2′S,4S)-2′-{(2R)-3-[(7-{[tert-butyl(dimethyl)silyl]oxy}-6,7-dihydro-5H-cyclopenta[b]pyridin-4-yl)oxy]-2-methylpropyl}-4-(3-chloroanilino)-2′,3′-dihydrospiro[cyclohexane-1,1′-indene]-4-carboxylate, diastereoisomer 1

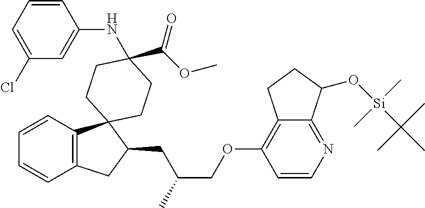 [SMILES: COC(=O)[C@]1(Nc2cccc(Cl)c2)CC[C@@]2(CC1)c1ccccc1C[C@@H]2C[C@@H](C)COc1ccnc2c1CCC2O[Si](C)(C)C(C)(C)C]

Using General Procedure 31a and Preparation 10b1 as the appropriate alcohol and Example 608A as the appropriate aryl chloride, Example 608B was isolated as a colourless gum. LRMS calculated for C₄₀H₅₃ClN₂O₄Si: 688; found: 689 (M+H).

### Example 608C methyl (1r,2′S,4S)-4-(3-chloroanilino)-2′-{(2R)-3-[(7-hydroxy-6,7-dihydro-5H-cyclopenta[b]pyridin-4-yl)oxy]-2-methylpropyl}-2′,3′-dihydrospiro[cyclohexane-1,1′-indene]-4-carboxylate, diastereoisomer 1

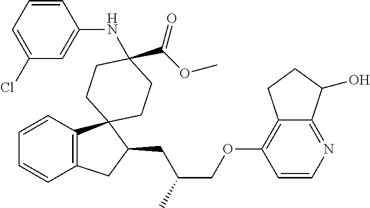 [SMILES: COC(=O)[C@]1(Nc2cccc(Cl)c2)CC[C@@]2(CC1)c1ccccc1C[C@@H]2C[C@@H](C)COc1ccnc2c1CCC2O]

Using General Procedure 29 and Example 608B as the appropriate silyl derivative, Example 608C was isolated as a colourless solid. LRMS calculated for C₃₄H₃₉ClN₂O₄: 574; found: 575 (M+H).

### Example 608 (1r,2′S,4S)-4-(3-chloroanilino)-2′-{(2R)-3-[(7-hydroxy-6,7-dihydro-5H-cyclopenta[b]pyridin-4-yl)oxy]-2-methylpropyl}-2′,3′-dihydrospiro[cyclohexane-1,1′-indene]-4-carboxylic acid, diastereoisomer 1

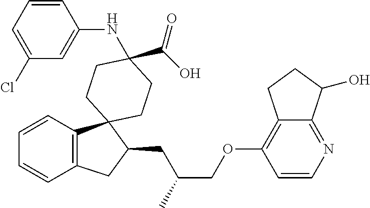 [SMILES: C[C@@H](COc1ccnc2c1CCC2O)C[C@H]1Cc2ccccc2[C@]12CC[C@@](Nc1cccc(Cl)c1)(C(=O)O)CC2]

Using General Procedure 33b and Example 608C as the appropriate ester, Example 608 was isolated as a white powder. LRMS calculated for C₃₃H₃₇ClN₂O₄: 560; found: 561 (M+H). ¹H NMR (400 MHz, DMSO-d₆) δ ppm: 8.54 (d, J=6.4 Hz, 1H), 7.43-7.37 (m, 1H), 7.34-7.27 (m, 1H), 7.23-7.12 (m, 3H), 7.06 (t, J=8.0 Hz, 1H), 6.63 (t, J=2.1 Hz, 1H), 6.58-6.51 (m, 2H), 5.87 (br s, 1H), 5.23-5.15 (m, 1H), 4.23-4.09 (m, 2H), 3.00 (dd, J=15.6, 7.0 Hz, 1H), 2.88 (ddd, J=16.2, 8.7, 5.0 Hz, 1H), 2.74-2.63 (m, 1H), 2.58-2.39 (m, 3H), 2.18-1.82 (m, 7H), 1.78-1.67 (m, 1H), 1.54-1.31 (m, 4H), 1.05 (d, J=6.7 Hz, 3H).

### Example 609B methyl (1r,2′S,4S)-2′-{(2R)-3-[(7-{[tert-butyl(dimethyl)silyl]oxy}-6,7-dihydro-5H-cyclopenta[b]pyridin-4-yl)oxy]-2-methylpropyl}-4-(3-chloroanilino)-2′,3′-dihydrospiro[cyclohexane-1,1′-indene]-4-carboxylate, diastereoisomer 2

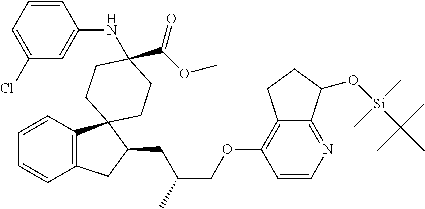 [SMILES: COC(=O)[C@]1(Nc2cccc(Cl)c2)CC[C@@]2(CC1)c1ccccc1C[C@@H]2C[C@@H](C)COc1ccnc2c1CCC2O[Si](C)(C)C(C)(C)C]

Using General Procedure 31a and Preparation 10b1 as the appropriate alcohol and Example 609A as the appropriate aryl chloride, Example 609B was isolated as a colourless gum. LRMS calculated for C₄₀H₅₃ClN₂O₄Si: 688; found: 689 (M+H).

### Example 609C methyl (1r,2′S,4S)-4-(3-chloroanilino)-2′-{(2R)-3-[(7-hydroxy-6,7-dihydro-5H-cyclopenta[b]pyridin-4-yl)oxy]-2-methylpropyl}-2′,3′-dihydrospiro[cyclohexane-1,1′-indene]-4-carboxylate, diastereoisomer 2

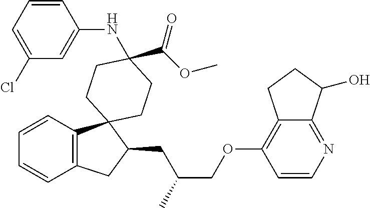 [SMILES: COC(=O)[C@]1(Nc2cccc(Cl)c2)CC[C@@]2(CC1)c1ccccc1C[C@@H]2C[C@@H](C)COc1ccnc2c1CCC2O]

Using General Procedure 29 and Example 609B as the appropriate silyl derivative, Example 609C was isolated as a colourless solid. LRMS calculated for C₃₄H₃₉ClN₂O₄: 574; found: 575 (M+H).

### Example 609 (1r,2′S,4S)-4-(3-chloroanilino)-2′-{(2R)-3-[(7-hydroxy-6,7-dihydro-5H-cyclopenta[b]pyridin-4-yl)oxy]-2-methylpropyl}-2′,3′-dihydrospiro[cyclohexane-1,1′-indene]-4-carboxylic acid, diastereoisomer 2

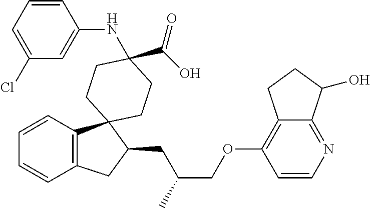 [SMILES: C[C@@H](COc1ccnc2c1CCC2O)C[C@H]1Cc2ccccc2[C@]12CC[C@@](Nc1cccc(Cl)c1)(C(=O)O)CC2]

Using General Procedure 33b and Example 609C as the appropriate ester, Example 609 was isolated as a white powder. LRMS calculated for C₃₃H₃₇ClN₂O₄: 560; found: 561 (M+H). ¹H NMR (400 MHz, DMSO-d₆) δ ppm: 8.27 (d, J=5.6 Hz, 1H), 7.42-7.36 (m, 1H), 7.23-7.11 (m, 3H), 7.05 (t, J=8.1 Hz, 1H), 6.85 (d, J=5.7 Hz, 1H), 6.62 (t, J=2.1 Hz, 1H), 6.57-6.52 (m, 2H), 5.28 (d, J=5.6 Hz, 1H), 4.92-4.86 (m, 1H), 3.99-3.87 (m, 2H), 2.99 (dd, J=15.6, 7.2 Hz, 1H), 2.81 (ddd, J=16.1, 8.7, 4.8 Hz, 1H), 2.63-2.39 (m, 3H), 2.34-2.24 (m, 1H), 2.17-1.67 (m, 8H), 1.53-1.29 (m, 4H), 1.03 (d, J=6.6 Hz, 3H).

### Example 610

### Example 610A 4-chloro-5-methyl-1-oxo-6,7-dihydro-5H-1λ⁵-cyclopenta[b]pyridine

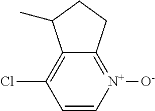

To a solution of Example 477D (1 g, 5.97 mmol, 1 eq) in DCM (20 mL) at 0° C., was added mCPBA (70% purity, 1.76 g, 7.16 mmol, 1.2 eq) in portions and the mixture was stirred at rt for 2 h. 2 M aq. NaOH solution (30 mL) was added and the mixture was stirred at rt for 1 h.

The mixture was partitioned between DCM and water, and the organic phase was washed with brine, dried (MgSO₄) and concentrated in vacuo to afford a racemate, Example 610A, isolated as a yellow oil (1.1 g, 5.97 mmol, 100%). LRMS calculated for C₉H₁₀ClNO: 183; found: 184 (M+H).

### Example 610B 4-chloro-5-methyl-6,7-dihydro-5H-cyclopenta[b]pyridin-7-ol

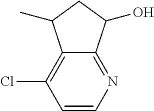

To a solution of Example 610A (1.1 g, 5.99 mmol, 1 eq) in DCM (20 mL), cooled to 0° C., was added TFAA (2.5 mL, 18 mmol, 3 eq) dropwise. The mixture was stirred at rt for 4 h. 2 M aq. NaOH solution (30 mL) was added and the mixture was stirred vigorously for 1 h. The mixture was partitioned between DCM and water, and the organic phase was washed with brine, dried (MgSO₄) and concentrated in vacuo. Purification by automated flash chromatography (CombiFlash Rf, 24 g RediSep™ silica cartridge) eluting with a gradient of 25-75% EtOAc (3% TEA) in heptane afforded a racemic mixture of diastereoisomers, Example 610B as a yellow wax (595 mg, 3.24 mmol, 54%). LRMS calculated for C₉H₁₀ClNO: 183; found: 184 (M+H). ¹H NMR (400 MHz, DMSO-d₆) δ ppm: 8.41-8.37 (m, 1H), 7.39-7.34 (m, 1H), 5.57-5.51 (m, 1H), 5.18-5.10/4.91-4.86 (m, 1H), 3.44-3.34/3.30-3.20 (m, 1H), 2.59-2.51/2.15-1.99/1.63-1.55 (m, 2H), 1.38/1.24 (d, J=7.1 Hz, 3H).

### Example 610C 4-chloro-7-[(4-methoxyphenyl)methoxy]-5-methyl-6,7-dihydro-5H-cyclopenta[b]pyridine

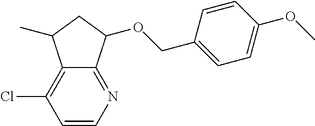 [SMILES: COc1ccc(COC2CC(C)c3c(Cl)ccnc32)cc1]

To an oven-dried flask was added Example 610B (210 mg, 1.14 mmol, 1 eq), PMB-Cl (185 μL, 1.37 mmol, 1.2 eq) and TBAI (42.2 mg, 114 μmol, 0.1 eq) in DMF (3 mL). NaH (60% dispersion; 91.5 mg, 2.29 mmol, 2 eq) was added in portions and the mixture was stirred at rt for 18 h. The mixture was partitioned between DCM and sat. aq. NaHCO₃ solution, and the organic phase was washed with brine, dried (PTFE phase separator) and concentrated in vacuo. Purification by automated flash chromatography (CombiFlash Rf, 12 g RediSep™ silica cartridge) eluting with a gradient of 0-34% EtOAc in heptane afforded a racemic mixture of diastereoisomers, Example 610C, isolated as a colourless oil (259 mg, 0.85 mmol, 75%). LRMS calculated for C₁₇H₁₈ClNO₂: 303; found: 304 (M+H). ¹H NMR (400 MHz, DMSO-d₆) δ ppm: 8.46-8.41 (m, 1H), 7.47-7.41 (m, 1H), 7.32-7.24 (m, 2H), 6.94-6.88 (m, 2H), 5.10-4.63 (m, 3H), 3.75/3.74 (s, 3H), 3.50-3.40/3.36-3.26 (m, 1H), 2.56-2.46/2.28-2.19 (m, 1H), 2.14-2.06/1.85-1.78 (m, 1H), 1.37/1.27 (d, J=7.1 Hz, 3H).

### Example 610D (1r,2′S,4S)-4-(3-chloroanilino)-2′-[(2R)-3-({7-[(4-methoxyphenyl)methoxy]-5-methyl-6,7-dihydro-5H-cyclopenta[b]pyridin-4-yl}oxy)-2-methylpropyl]-2′,3′-dihydrospiro[cyclohexane-1,1′-indene]-4-carboxylic acid

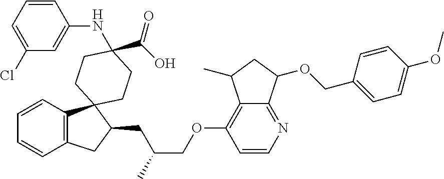 [SMILES: COc1ccc(COC2CC(C)c3c(OC[C@H](C)C[C@H]4Cc5ccccc5[C@]45CC[C@@](Nc4cccc(Cl)c4)(C(=O)O)CC5)ccnc32)cc1]

Using General Procedure 31c and Preparation 10b1 as the appropriate alcohol and Example 610C as the appropriate aryl chloride, a mixture of 4 diastereoisomers, Example 610D was isolated as an off-white powder. LRMS calculated for C₄₂H₄₇ClN₂O₅: 694; found: 695 (M+H).

### Example 610 (1r,2′S,4S)-4-(3-chloroanilino)-2′-{(2R)-3-[(7-hydroxy-5-methyl-6,7-dihydro-5H-cyclopenta[b]pyridin-4-yl)oxy]-2-methylpropyl}-2′,3′-dihydrospiro[cyclohexane-1,1′-indene]-4-carboxylic acid

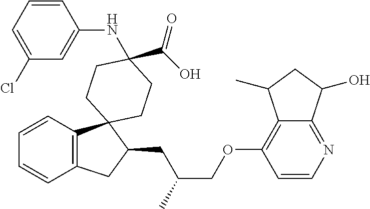 [SMILES: CC1CC(O)c2nccc(OC[C@H](C)C[C@H]3Cc4ccccc4[C@]34CC[C@@](Nc3cccc(Cl)c3)(C(=O)O)CC4)c21]

Using General Procedure 28b and Example 610D as the appropriate PMB derivative, a mixture of 4 diastereoisomers, Example 610 was isolated as a white powder. LRMS calculated for C₃₄H₃₉ClN₂O₄: 574; found: 575 (M+H). ¹H NMR (400 MHz, DMSO-d₆) δ ppm: 8.30-8.25 (m, 1H), 7.42-7.34 (m, 1H), 7.24-7.11 (m, 3H), 7.09-7.01 (m, 1H), 6.88-6.82 (m, 1H), 6.66-6.61 (m, 1H), 6.58-6.51 (m, 2H), 5.42-5.11 (br m, 1H), 5.01-4.92/4.84-4.75 (m, 1H), 4.00-3.86 (m, 2H), 3.40-2.95 (m, 2H), 2.59-1.94 (m, 7H), 1.94-1.09 (m, 11H), 1.09-0.99 (m, 3H).

### Example 611

### Example 611A 4-chloro-6,6-dimethyl-6,7-dihydro-5H-cyclopenta[b]pyridine

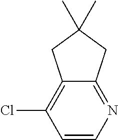

To an oven-dried flask was added 3,3-dimethylcyclopentan-1-one (1.67 mL, 13.4 mmol, 1 eq) in EtOH (35 mL), followed by prop-2-yn-1-amine (1.84 mL, 26.8 mmol, 2 eq) and NaAuCl₄×2H₂O (106 mg, 0.27 mmol, 0.02 eq). The flask was sealed and heated at 80° C. for 24 h. The mixture was allowed to cool to rt, filtered through celite, washed with EtOH and concentrated in vacuo to afford an intermediate that was taken up in DCM (50 mL). After cooling to 0° C., mCPBA (70% purity, 7.95 g, 32.3 mmol, 2.4 eq) was added in portions and then the mixture was stirred at rt for 4 h. 2 M aq. NaOH solution was added and the mixture was stirred at rt for 1 h. The mixture was partitioned between DCM and water, and the organic phase was washed with brine, dried (MgSO₄) and concentrated in vacuo. Purification by automated flash chromatography (CombiFlash Rf, 24 g RediSep™ silica cartridge) eluting with a gradient of 0-8% MeOH in DCM afforded an intermediate that was added dropwise to rapidly stirring POCl₃ (2.36 mL, 25.3 mmol) at rt. The mixture was heated at 100° C. for 72 h and then allowed to cool to rt and added dropwise to a rapidly stirring solution of ice/water. The mixture was partitioned between DCM and aq. NH₃ solution, and the organic phase was washed with brine, dried (MgSO₄) and concentrated in vacuo. Purification by automated flash chromatography (CombiFlash Rf, 12 g RediSep™ silica cartridge) eluting with a gradient of 0-33% EtOAc (3% TEA) in heptane afforded Example 611A as a yellow oil (72 mg, 0.4 mmol, 3%). LRMS calculated for C₁₀H₁₂ClN: 181; found: 182 (M+H). ¹H NMR (400 MHz, DMSO-d₆) δ ppm: 8.27-8.24 (m, 1H), 7.29-7.25 (m, 1H), 2.84 (s, 2H), 2.77 (s, 2H), 1.15 (s, 6H).

### Example 611B (1r,2′S,4S)-4-(3-chloroanilino)-2′-[(2R)-3-hydroxy-2-methylpropyl]-2′,3′-dihydrospiro[cyclohexane-1,1′-indene]-4-carboxylic acid

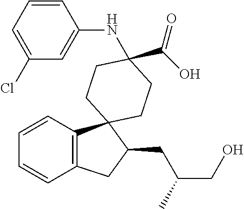 [SMILES: C[C@@H](CO)C[C@H]1Cc2ccccc2[C@]12CC[C@@](Nc1cccc(Cl)c1)(C(=O)O)CC2]

Using General Procedure 33b and Preparation 10b1 as the appropriate ester, Example 611B was isolated as a white solid. LRMS calculated for C₂₅H₃₀ClNO₃: 427; found: 428 (M+H).

### Example 611 (1r,2′S,4S)-4-(3-chloroanilino)-2′-{(2R)-3-[(6,6-dimethyl-6,7-dihydro-5H-cyclopenta[b]pyridin-4-yl)oxy]-2-methylpropyl}-2′,3′-dihydrospiro[cyclohexane-1,1′-indene]-4-carboxylic acid

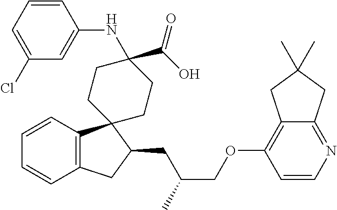 [SMILES: C[C@@H](COc1ccnc2c1CC(C)(C)C2)C[C@H]1Cc2ccccc2[C@]12CC[C@@](Nc1cccc(Cl)c1)(C(=O)O)CC2]

Using General Procedure 31c and Example 611B as the appropriate alcohol and Example 611A as the appropriate aryl chloride, Example 611 was isolated as a cream powder. LRMS calculated for C₃₅H₄₁ClN₂O₃: 572; found: 573 (M+H). ¹H NMR (400 MHz, DMSO-d₆) δ ppm: 8.18 (d, J=5.8 Hz, 1H), 7.41-7.34 (m, 1H), 7.24-7.11 (m, 3H), 7.05 (t, J=8.1 Hz, 1H), 6.82 (d, J=5.8 Hz, 1H), 6.62 (t, J=2.1 Hz, 1H), 6.58-6.53 (m, 2H), 3.93 (d, J=6.2 Hz, 2H), 2.98 (dd, J=15.6, 7.1 Hz, 1H), 2.71 (s, 2H), 2.62-2.38 (m, 4H), 2.19-2.07 (m, 2H), 2.05-1.80 (m, 4H), 1.78-1.66 (m, 1H), 1.53-1.23 (m, 4H), 1.11 (s, 3H), 1.09 (s, 3H), 1.02 (d, J=6.6 Hz, 3H).

### Example 612 and Example 613

### Example 612A 5-ethylidene-6,7-dihydro-5H-cyclopenta[b]pyridine

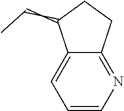

Ethyltriphenylphosphonium bromide (10.87 g, 29.29 mmol, 1.3 eq) was suspended in THE (80 mL) and the mixture cooled to 0° C. under N₂. nBuLi (12.74 mL, 2.3 M in hexanes, 29.29 mmol, 1.3 eq) was added dropwise and the mixture was stirred at 0° C. for 40 min. 6,7-dihydro-5H-cyclopenta[b]pyridin-5-one (3.00 g, 22.53 mmol, 1 eq) in THE (20 mL) was added dropwise at 0° C. and the mixture was allowed to warm to rt and stirred for 18 h. The mixture was cooled to 0° C. and quenched by the careful addition of sat. aq. NaHCO₃ solution. Excess THE was removed in vacuo, and the aq. phase was extracted with EtOAc. The organic phase was dried (MgSO₄) and concentrated in vacuo. Purification by automated flash chromatography (Combiflash Rf, Silica 40 g, RediSep™ cartridge) eluting with a gradient of 0-40% EtOAc (3% TEA) in heptane afforded a mixture of cis and trans isomers, Example 612A, isolated as a mixture of colourless oil and white crystals (1.76 g, 12.12 mmol, 54%). ¹H NMR (400 MHz, DMSO-d₆) δ ppm: 8.36-8.27 (m, 1H), 7.95-7.91/7.85-7.80 (m, 1H), 7.24-7.19/7.19-7.12 (m, 1H), 6.18-6.10/5.79-5.71 (m, 1H), 3.03-2.91 (m, 2H), 2.74-2.65 (m, 2H), 1.97-1.91/1.81-1.74 (m, 3H).

### Example 612B 5-ethyl-6,7-dihydro-5H-cyclopenta[b]pyridine

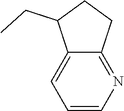

To a solution of Example 612A (1.76 g, 12.12 mmol, 1 eq) in EtOH (50 mL) was added 10% Pd/C (200 mg) under N₂ atmosphere. The mixture was evacuated and backfilled with N₂ (×3), then evacuated and backfilled with H₂ and shaken at rt under an atmosphere of H₂ for 3 h. The mixture was filtered through celite, washed with EtOH and the filtrate concentrated in vacuo to afford a racemate, Example 612B, isolated as a colourless oil (1.73 g, 11.75 mmol, 97%). LRMS calculated for C₁₀H₁₃N: 147; found: 148 (M+H). ¹H NMR (400 MHz, DMSO-d₆) δ ppm: 8.31-8.26 (m, 1H), 7.61-7.57 (m, 1H), 7.15-7.10 (m, 1H), 3.10-3.00 (m, 1H), 2.96-2.79 (m, 2H), 2.31-2.20 (m, 1H), 1.90-1.78 (m, 1H), 1.72-1.60 (m, 1H), 1.48-1.35 (m, 1H), 0.94 (t, J=7.4 Hz, 3H).

### Example 612C 5-ethyl-1-oxo-6,7-dihydro-5H-1λ⁵-cyclopenta[b]pyridine

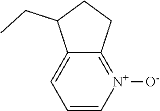

mCPBA (75% purity, 2.84 g, 12.34 mmol, 1.05 eq) was added portionwise to a stirred solution of Example 612B (1.73 g, 11.75 mmol, 1 eq) in DCM and the mixture was stirred at rt for 3 h. The mixture was partitioned between DCM and 1 M aq. NaOH solution. The aq. phase was extracted with DCM and the combined organic extracts were dried (MgSO₄) and concentrated in vacuo to afford a racemate, Example 612C, isolated as a yellow oil. LRMS calculated for C₁₀H₁₃NO: 163; found: 164 (M+H). ¹H NMR (400 MHz, DMSO-d₆) δ ppm: 8.07-8.02 (m, 1H), 7.27-7.19 (m, 2H), 3.21-3.12 (m, 1H), 3.02-2.92 (m, 1H), 2.91-2.81 (m, 1H), 2.32-2.21 (m, 1H), 1.88-1.66 (m, 2H), 1.51-1.39 (m, 1H), 0.92 (t, J=7.4 Hz, 3H).

### Example 612D 4-chloro-5-ethyl-6,7-dihydro-5H-cyclopenta[b]pyridine

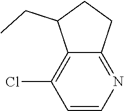

Example 612C (1.96 g, 12.01 mmol, 1 eq) was heated at 100° C. for 48 h in POCl₃ (11.19 mL, 120.09 mmol, 10 eq). The mixture was allowed to cool to rt and then added dropwise to a rapidly stirred mixture of ice/water. The solution was basified to pH 11 with aq. NH₃ solution and then extracted with DCM. The organic phase was washed with brine, dried (MgSO₄) and concentrated in vacuo. Purification by automated flash chromatography (Combiflash Rf, Silica 24 g, RediSep™ cartridge) eluting with a gradient of 0-28% EtOAc (3% TEA) in heptane afforded a racemate, Example 612D, isolated as a brown oil (1.17 g, 6.44 mmol, 54%). LRMS calculated for C₁₀H₁₂ClN: 181; found: 182 (M+H). ¹H NMR (400 MHz, DMSO-d₆) δ ppm: 8.29-8.25 (m, 1H), 7.28-7.24 (m, 1H), 3.29-3.21 (m, 1H), 3.14-3.03 (m, 1H), 2.92-2.82 (m, 1H), 2.25-2.13 (m, 1H), 1.96-1.87 (m, 1H), 1.83-1.72 (m, 1H), 1.57-1.45 (m, 1H), 0.91 (t, J=7.4 Hz, 3H).

### Example 612 (1r,2′S,4S)-4-(3-chloroanilino)-2′-[(2R)-3-{[(5R)-5-ethyl-6,7-dihydro-5H-cyclopenta[b]pyridin-4-yl]oxy}-2-methylpropyl]-2′,3′-dihydrospiro[cyclohexane-1,1′-indene]-4-carboxylic acid

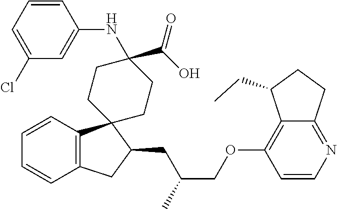 [SMILES: CC[C@@H]1CCc2nccc(OC[C@H](C)C[C@H]3Cc4ccccc4[C@]34CC[C@@](Nc3cccc(Cl)c3)(C(=O)O)CC4)c21]

and

### Example 613 (1r,2′S,4S)-4-(3-chloroanilino)-2′-[(2R)-3-{[(5S)-5-ethyl-6,7-dihydro-5H-cyclopenta[b]pyridin-4-yl]oxy}-2-methylpropyl]-2′,3′-dihydrospiro[cyclohexane-1,1′-indene]-4-carboxylic acid

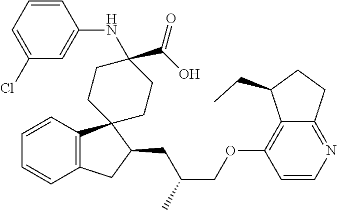 [SMILES: CC[C@H]1CCc2nccc(OC[C@H](C)C[C@H]3Cc4ccccc4[C@]34CC[C@@](Nc3cccc(Cl)c3)(C(=O)O)CC4)c21]

Using General procedure 31c and Preparation 10b1 as the appropriate indane and Example 612D as the appropriate aryl chloride a mixture of diastereoisomers was obtained. They were separated by chiral chromatography. Column: AD, 50 mm×500 mm, 20 μm, Eluents: 15:85 iPrOH/heptane. The diastereoisomer eluting earlier was hydrolyzed according to General procedure 33a to obtain Example 612. ¹H NMR (400 MHz, DMSO-d₆) δ ppm: 8.15 (d, 1H), 7.41-7.11 (m, 4H), 7.03 (t, 1H), 6.77 (d, 1H), 6.61 (t, 1H), 6.53 (m, 2H), 3.89 (m, 2H), 3.12 (m, 1H), 2.99/2.52 (dd+dd, 2H), 2.89/2.73 (m+dm, 2H), 2.48-1.29 (m, 8H), 2.13 (m, 1H), 2.11/1.71 (m+m, 2H), 1.99 (m, 1H), 1.71/1.35 (m+m, 2H), 1.51/1.31 (m+m, 2H), 1.01 (d, 3H), 0.67 (t, 3H). HRMS calculated for C₃₅H₄₁N₂O₃Cl: 572.2806; found: 573.2886 (M+H).

The diastereoisomer eluting later was hydrolyzed according to General procedure 33a to obtain Example 613. ¹H NMR (500 MHz, DMSO-d₆) δ ppm: 8.15 (d, 1H), 7.41-7.10 (m, 4H), 7.05 (t, 1H), 6.76 (d, 1H), 6.62 (t, 1H), 6.55 (dm, 1H), 6.53 (dm, 1H), 6.21 (br s, 1H), 3.93/3.86 (dd+dd, 2H), 3.09 (m, 1H), 2.99/2.52 (dd+dd, 2H), 2.89/2.73 (m+m, 2H), 2.49-1.34 (m, 8H), 2.15 (m, 1H), 2.08/1.70 (m+m, 2H), 1.98 (m, 1H), 1.79/1.40 (m+m, 2H), 1.44/1.33 (m+m, 2H), 1.03 (d, 3H), 0.76 (t, 3H). HRMS calculated for C₃₅H₄₁N₂O₃Cl: 572.2806; found: 573.2871 (M+H).

### Example 614 (1r,4r)-4-(3-chloroanilino)-2′-{3-[(6,7-dihydro-5H-cyclopenta[b]pyridin-4-yl)oxy]-2-methylbutyl}-2′,3′-dihydrospiro[cyclohexane-1,1′-indene]-4-carboxylic acid, diastereoisomer 1, enantiomer 2

and

### Example 615 (1r,4r)-4-(3-chloroanilino)-2′-{3-[(6,7-dihydro-5H-cyclopenta[b]pyridin-4-yl)oxy]-2-methylbutyl}-2′,3′-dihydrospiro[cyclohexane-1,1′-indene]-4-carboxylic acid, diastereoisomer 2, enantiomer 2

and

### Example 616 (1r,2′S,4S)-4-(3-chloroanilino)-2′-{(2R,3R)-3-[(6,7-dihydro-5H-cyclopenta[b]pyridin-4-yl)oxy]-2-methylbutyl}-2′,3′-dihydrospiro[cyclohexane-1,1′-indene]-4-carboxylic acid

and

### Example 617 (1r,4r)-4-(3-chloroanilino)-2′-{3-[(6,7-dihydro-5H-cyclopenta[b]pyridin-4-yl)oxy]-2-methylbutyl}-2′,3′-dihydrospiro[cyclohexane-1,1′-indene]-4-carboxylic acid, diastereoisomer 3, enantiomer 2

### Example 614A 2-(4-methoxyphenyl)-4,5-dimethyl-1,3-dioxane

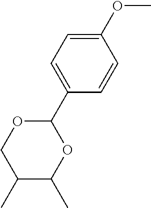 [SMILES: COc1ccc(C2OCC(C)C(C)O2)cc1]

1-(dimethoxymethyl)-4-methoxy-benzene (6.01 g, 33.0 mmol) was dissolved in dry DCM (60 mL). 2-methylbutane-1,3-diol (3.1 g, 30 mmol) and PPTS (503 mg, 2.0 mmol) were added to the mixture and stirred at rt until no further conversion was observed. The reaction was diluted with 10% aq. K₂CO₃ solution and stirred at rt for 30 min. The layers were separated and the aq. layer was extracted with DCM. The combined organic layers were dried over Na₂SO₄, filtered and the filtrate was concentrated under reduced pressure to obtain Example 614A as a racemic mixture of diastereoisomers. MS (EI, 70 eV) m/z (% relative intensity, [ion]): 55 (13), 77 (16), 108 (17), 135 (100), 136 (64), 221 (23), 222 (14, [M+]).

### Example 614B 3-[(4-methoxyphenyl)methoxy]-2-methylbutan-1-ol, diastereoisomers 1 enantiomer 1

and

### Example 614C 3-[(4-methoxyphenyl)methoxy]-2-methylbutan-1-ol, diastereoisomers 2 enantiomer 1

and

### Example 614D 3-[(4-methoxyphenyl)methoxy]-2-methylbutan-1-ol, diastereoisomers 1 enantiomer 2 and diastereoisomer 2 enantiomer 2

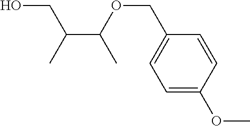 [SMILES: COc1ccc(COC(C)C(C)CO)cc1]

Example 614A (4.69 g, 21.1 mmol) was dissolved in DCM (85 mL) and cooled to 0° C. DIBAL-H (23.3 mL, 1 M solution in DCM) was added dropwise at 0° C. The reaction mixture was allowed to warm to rt and stirred for 1 h. The mixture was cooled to 0° C., MeOH (10 mL) was added dropwise at 0° C., then water (10 mL) was added. The mixture was stirred at rt for 1 h, then it was diluted with water (30 mL). The layers were separated. The organic layer was dried over Na₂SO₄, filtered and the filtrate was concentrated under reduced pressure. The diastereoisomers were partially separated by chiral chromatography. Column: AS-V, 100×500 mm, 20 μm, Eluents: 3:97 iPrOH/heptane. The obtained fractions were characterized by chiral HPLC (column: AS-H, 250×4.6 mm, 5 μm, eluents: 3:97 iPrOH/heptane).

A single diastereoisomer eluting firstly was collected as Example 614B. rt: 19.35 min.

A single diastereoisomer eluting secondly was collected as Example 614C. rt: 23.39 min.

A mixture of two diastereoisomers eluting thirdly was collected as Example 614D. rt: 28.82 and 31.41 min.

### Example 614E 1-{[(4-bromo-3-methylbutan-2-yl)oxy]methyl}-4-methoxybenzene

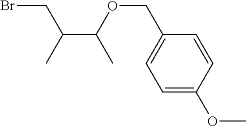 [SMILES: COc1ccc(COC(C)C(C)CBr)cc1]

Using General procedure 3 and Example 614D as the appropriate alcohol Example 614E was obtained as a mixture of diastereoisomers. ¹H NMR (500 MHz, DMSO-d₆) δ ppm: 7.25 (m, 2H), 6.90 (m, 2H), 4.48 (d, 1H), 4.30 (d, 1H), 3.74 (s, 3H), 3.57 (m, 2H), 3.42 (m, 1H), 1.88 (m, 1H), 1.11 (d, 3H), 0.97/0.94 (d, 3H). HRMS calculated for C₁₃H₁₉BrO₂: 286.0569; found: 286.05610 and 286.0558 (M+H).

### Example 614F bromido{3-[(4-methoxyphenyl)methoxy]-2-methylbutyl}zinc

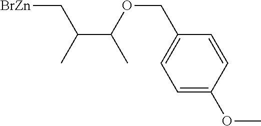

Using General procedure 4 and Example 614E as the appropriate bromo compound, Example 614F was obtained as a mixture of diastereoisomers.

### Example 614G methyl (1r,4r)-4-(3-chloroanilino)-2′-{3-[(4-methoxyphenyl)methoxy]-2-methylbutyl}spiro[cyclohexane-1,1′-indene]-4-carboxylate

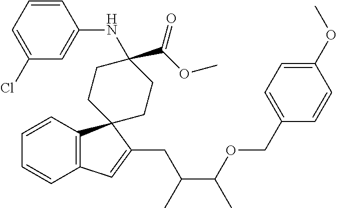 [SMILES: COc1ccc(COC(C)C(C)CC2=Cc3ccccc3[C@]23CC[C@@](Nc2cccc(Cl)c2)(C(=O)OC)CC3)cc1]

Using General procedure 27a and Preparation 6c as the appropriate 2-bromo-indene derivative and Example 614F as the appropriate Zn reagent, a mixture of diastereoisomers, Example 614G was obtained as a yellow glass (1.03 g, 1.79 mmol, 80%). LRMS calculated for C₃₅H₄₀ClNO₄: 573; found: 574 (M+H). ¹H NMR (400 MHz, DMSO-d₆) δ ppm: 7.69-7.62 (m, 1H), 7.30-7.18 (m, 4H), 7.14-7.06 (m, 2H), 6.88-6.77 (m, 2H), 6.68-6.63 (m, 1H), 6.63-6.58 (m, 1H), 6.53-6.39 (m, 3H), 4.51-4.44 (m, 1H), 4.39-4.28 (m, 1H), 3.74-3.68 (m, 6H), 3.54-3.42 (m, 1H), 2.48-1.90 (m, 9H), 1.15/1.10 (d, J=6.2 Hz, 3H), 0.92-0.81 (m, 5H).

### Example 614H methyl (1r,4r)-4-(3-chloroanilino)-2′-(3-hydroxy-2-methylbutyl)spiro[cyclohexane-1,1′-indene]-4-carboxylate

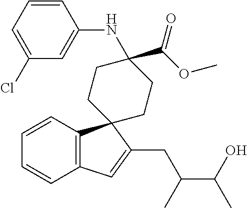 [SMILES: COC(=O)[C@]1(Nc2cccc(Cl)c2)CC[C@]2(CC1)C(CC(C)C(C)O)=Cc1ccccc12]

Using General procedure 28b and Example 614G as the appropriate PMB derivative, a mixture of diastereoisomers, Example 614H, was obtained as a white foam (744 mg, 1.64 mmol, 94%). LRMS calculated for C₂₇H₃₂NO₃Cl: 453; found: 454 (M+H). ¹H NMR (400 MHz, DMSO-d₆) δ ppm: 7.68-7.63 (m, 1H), 7.32-7.26 (m, 1H), 7.26-7.19 (m, 1H), 7.14-7.07 (m, 2H), 6.66-6.62 (m, 1H), 6.62-6.58 (m, 1H), 6.51-6.41 (m, 3H), 4.47/4.39 (d, J=4.7 Hz, 1H), 3.70 (s, 3H), 3.69-3.62/3.59-3.50 (m, 1H), 2.48-2.35 (m, 3H), 2.27-1.80 (m, 6H), 1.11-1.05 (m, 3H), 0.94-0.81 (m, 5H).

### Example 6141 methyl (1r,4r)-4-(3-chloroanilino)-2′-(3-hydroxy-2-methylbutyl)-2′,3′-dihydrospiro[cyclohexane-1,1′-indene]-4-carboxylate

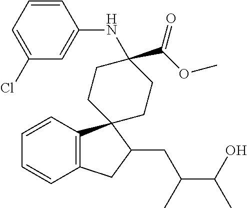

Using General procedure 19 and Example 614H (744 mg, 1.64 mmol, 1 eq) as the appropriate indene, a mixture of 4 diastereoisomers, Example 6141, was obtained as a white foam (682 mg, 1.5 mmol, 91%). LRMS calculated for C₂₇H₃₄ClNO₃: 455; found: 456 (M+H). ¹H NMR (400 MHz, DMSO-d₆) δ ppm: 7.43-7.33 (m, 1H), 7.25-7.03 (m, 4H), 6.63-6.53 (m, 2H), 6.50-6.42 (m, 1H), 6.34-6.28 (m, 1H), 4.36-4.19 (m, 1H), 3.71-3.38 (m, 4H), 3.03-2.88 (m, 1H), 2.64-2.38 (m, 2H), 2.21-1.13 (m, 10H), 1.13-0.73 (m, 7H).

### Example 614 (1r,4r)-4-(3-chloroanilino)-2′-{3-[(6,7-dihydro-5H-cyclopenta[b]pyridin-4-yl)oxy]-2-methylbutyl}-2′,3′-dihydrospiro[cyclohexane-1,1′-indene]-4-carboxylic acid, diastereoisomer 1, enantiomer 2

and

### Example 615 (1r,4r)-4-(3-chloroanilino)-2′-{3-[(6,7-dihydro-5H-cyclopenta[b]pyridin-4-yl)oxy]-2-methylbutyl}-2′,3′-dihydrospiro[cyclohexane-1,1′-indene]-4-carboxylic acid, diastereoisomer 2, enantiomer 2

and

### Example 617 (1r,4r)-4-(3-chloroanilino)-2′-{3-[(6,7-dihydro-5H-cyclopenta[b]pyridin-4-yl)oxy]-2-methylbutyl}-2′,3′-dihydrospiro[cyclohexane-1,1′-indene]-4-carboxylic acid, diastereoisomer 3, enantiomer 2

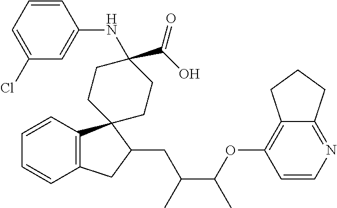

and

### Example 616 (1r,2′S,4S)-4-(3-chloroanilino)-2′-{(2R,3R)-3-[(6,7-dihydro-5H-cyclopenta[b]pyridin-4-yl)oxy]-2-methylbutyl}-2′,3′-dihydrospiro[cyclohexane-1,1′-indene]-4-carboxylic acid

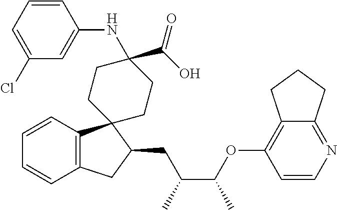 [SMILES: C[C@H](C[C@H]1Cc2ccccc2[C@]12CC[C@@](Nc1cccc(Cl)c1)(C(=O)O)CC2)[C@@H](C)Oc1ccnc2c1CCC2]

Using General procedure 31c and Example 6141 as the appropriate indane and 4-chloro-6,7-dihydro-5H-cyclopenta[b]pyridine as the appropriate aryl chloride, followed by General procedure 17a, a mixture of diastereoisomers was obtained. They were separated by chiral chromatography. Column: AD, 50 mm×500 mm, 20 μm, Eluents: 10:90 EtOH/heptane. The diastereoisomer eluting firstly was hydrolyzed according to General procedure 33a to obtain Example 614. ¹H NMR (500 MHz, DMSO-d₆) δ ppm: 12.71 (br s, 1H), 8.12 (d, 1H), 7.41-7.10 (m, 4H), 7.04 (t, 1H), 6.76 (d, 1H), 6.62 (t, 1H), 6.53 (m, 2H), 6.23 (br s, 1H), 4.44 (m, 1H), 2.97/2.54 (dd+dd, 2H), 2.88-1.88 (m, 6H), 2.51-1.29 (m, 8H), 2.05 (m, 1H), 1.80 (m, 1H), 1.42/1.26 (m+m, 2H), 1.21 (d, 3H), 0.95 (d, 3H). HRMS calculated for C₃₄H₃₉N₂O₃Cl: 558.2649; found: 559.2711 (M+H).

The diastereoisomer eluting secondly was hydrolyzed according to General procedure 33a to obtain Example 615. ¹H NMR (500 MHz, DMSO-d₆) δ ppm: 12.67 (br s, 1H), 8.13 (d, 1H), 7.44-7.10 (m, 4H), 7.03 (t, 1H), 6.79 (d, 1H), 6.61 (t, 1H), 6.54 (dm, 1H), 6.52 (dm, 1H), 6.21 (br s, 1H), 4.59 (m, 1H), 2.98/2.55 (dd+dd, 2H), 2.89-1.89 (m, 6H), 2.50-1.20 (m, 8H), 2.13 (m, 1H), 1.97 (m, 1H), 1.65/1.15 (m+m, 2H), 1.20 (d, 3H), 0.97 (d, 3H). HRMS calculated for C₃₄H₃₉N₂O₃Cl: 558.2649; found: 559.2730 (M+H).

The diastereoisomer eluting thirdly was hydrolyzed according to General procedure 33a to obtain Example 616. ¹H NMR (500 MHz, DMSO-d₆) δ ppm: 12.65 (br s, 1H), 8.13 (d, 1H), 7.38-7.09 (m, 4H), 7.02 (t, 1H), 6.79 (d, 1H), 6.60 (t, 1H), 6.54 (dm, 1H), 6.51 (dm, 1H), 6.24 (br s, 1H), 4.50 (m, 1H), 2.91-1.93 (m, 6H), 2.89/2.34 (dd+dd, 2H), 2.48-1.32 (m, 8H), 2.05 (m, 1H), 1.80 (m, 1H), 1.30 (m, 2H), 1.21 (d, 3H), 0.99 (d, 3H). HRMS calculated for C₃₄H₃₉N₂O₃Cl: 558.2649; found: 559.2723 (M+H).

The diastereoisomer eluting fourthly was hydrolyzed according to General procedure 33a to obtain Example 617. ¹H NMR (500 MHz, DMSO-d₆) δ ppm: 12.67 (br s, 1H), 8.08 (d, 1H), 7.38-7.10 (m, 4H), 7.03 (t, 1H), 6.78 (d, 1H), 6.59 (t, 1H), 6.55 (dd, 1H), 6.49 (dd, 1H), 6.08 (br s, 1H), 4.64 (m, 1H), 2.92/2.52 (dd+dd, 2H), 2.73/2.64 (m+m, 2H), 2.65/2.57 (m+m, 2H), 2.37-1.17 (m, 8H), 2.01 (m, 1H), 1.89 (m, 1H), 1.81/1.71 (m+m, 2H), 1.70/1.12 (m+m, 2H), 1.25 (d, 3H), 1.03 (d, 3H). HRMS calculated for C₃₄H₃₉N₂O₃Cl: 558.2649; found: 559.2728 (M+H).

### Example 618

### Example 618A 4-chloro-7-methyl-6,7-dihydro-5H-cyclopenta[b]pyridin-7-ol

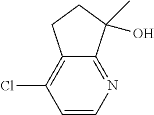

To a solution of 4-chloro-5,6-dihydro-7H-cyclopenta[b]pyridin-7-one (400 mg, 2.39 mmol, 1 eq) in THE (8 mL) cooled to 0° C. was added MeMgBr (2.39 mL, 3 M, 7.16 mmol, 3 eq) dropwise and the mixture was warmed to rt and stirred for 48 h. The mixture was cooled to 0° C., quenched with sat. aq. NH₄Cl solution and partitioned between EtOAc and water. The organic phase was washed with brine, dried (MgSO₄) and concentrated in vacuo. Purification by automated flash chromatography (CombiFlash Rf, 24 g RediSep™ silica cartridge) eluting with a gradient of 0-48% EtOAc in heptane afforded a racemate, Example 618A, isolated as a green solid (152 mg, 0.83 mmol, 35%). LRMS calculated for C₉H₁₀ClNO: 183; found: 184 (M+H).

### Example 618B 4-chloro-7-[(4-methoxyphenyl)methoxy]-7-methyl-6,7-dihydro-5H-cyclopenta[b]pyridine

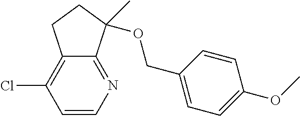 [SMILES: COc1ccc(COC2(C)CCc3c(Cl)ccnc32)cc1]

To a solution of Example 618A (207 mg, 1.13 mmol, 1 eq) in DMF (4 mL) cooled to 0° C. was added PMB-Cl (0.18 mL, 1.35 mmol, 1.2 eq) and TBAI (0.02 mL, 0.11 mmol, 0.1 eq), followed by NaH (60% dispersion; 54 mg, 2.25 mmol, 2 eq). The mixture was warmed to rt and stirred for 18 h. The mixture was quenched with sat. aq. NH₄Cl solution and partitioned between EtOAc and water. The phases were separated, and the organic phase was washed with brine, dried (MgSO₄) and concentrated in vacuo. Purification by automated flash chromatography (CombiFlash Rf, 12 g RediSep™ silica cartridge) eluting with a gradient of 0-50% EtOAc in heptane afforded a racemate, Example 618B, isolated as a yellow oil (121 mg, 0.4 mmol, 35%). LRMS calculated for C₁₇H₁₈ClNO₂: 303; found: 304 (M+H).

### Example 618C (1r,2′S,4S)-4-(3-chloroanilino)-2′-[(2R)-3-({7-[(4-methoxyphenyl)methoxy]-7-methyl-6,7-dihydro-5H-cyclopenta[b]pyridin-4-yl}oxy)-2-methylpropyl]-2′,3′-dihydrospiro[cyclohexane-1,1′-indene]-4-carboxylic acid

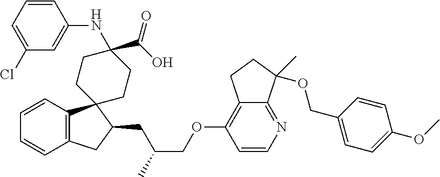 [SMILES: COc1ccc(COC2(C)CCc3c(OC[C@H](C)C[C@H]4Cc5ccccc5[C@]45CC[C@@](Nc4cccc(Cl)c4)(C(=O)O)CC5)ccnc32)cc1]

Using General Procedure 31c and Example 611B as the appropriate alcohol and Example 618B as the appropriate aryl chloride, a mixture of diastereoisomers, Example 618C, was isolated as a colourless gum. LRMS calculated for C₄₂H₄₇ClN₂O₅: 694; found: 695 (M+H).

### Example 618 (1r,2′S,4S)-4-(3-chloroanilino)-2′-{(2R)-3-[(7-hydroxy-7-methyl-6,7-dihydro-5H-cyclopenta[b]pyridin-4-yl)oxy]-2-methylpropyl}-2′,3′-dihydrospiro[cyclohexane-1,1′-indene]-4-carboxylic acid

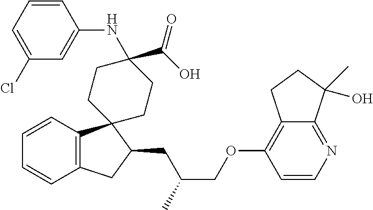 [SMILES: C[C@@H](COc1ccnc2c1CCC2(C)O)C[C@H]1Cc2ccccc2[C@]12CC[C@@](Nc1cccc(Cl)c1)(C(=O)O)CC2]

Using General Procedure 28b and Example 618C as the appropriate PMB derivative, a mixture of diastereoisomers, Example 618, was isolated as a white powder. LRMS calculated for C₃₄H₃₉ClN₂O₄: 574; found: 575 (M+H). ¹H NMR (400 MHz, DMSO-d₆) δ ppm: 8.26 (d, J=5.6 Hz, 1H), 7.42-7.36 (m, 1H), 7.23-7.11 (m, 3H), 7.09-7.01 (m, 1H), 6.84 (d, J=5.7 Hz, 1H), 6.64-6.61 (m, 1H), 6.58-6.52 (m, 2H), 4.95/4.94 (s, 1H), 3.99-3.87 (m, 2H), 3.03-2.94 (m, 1H), 2.82-2.38 (m, 4H), 2.18-1.81 (m, 8H), 1.78-1.66 (m, 1H), 1.53-1.30 (m, 7H), 1.03 (d, J=6.6 Hz, 3H).

### Example 619

### Example 619A 4-oxo-1H-4λ⁵-pyrrolo[3,2-b]pyridine

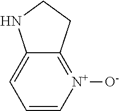

1H-pyrrolo[3,2-b]pyridine (1.0 eq.) was dissolved in DCM (4 mL/mmol) under N₂, then mCPBA (2.0 eq.) was added to the stirred solution portionwise at rt. The reaction mixture was stirred at rt under N₂ atmosphere until no further conversion was observed. The reaction mixture was diluted with Et₂O, and was concentrated under reduced pressure. The crude product was purified via flash chromatography using heptane and EtOAc as eluents to obtain Example 619A. ¹H NMR (400 MHz, DMSO-d₆) δ ppm: 11.79 (br s, 1H), 8.03 (dd, 1H), 7.59 (dd, 1H), 7.45 (dt, 1H), 7.08 (dd, 1H), 6.66 (d, 1H).

### Example 619B 7-chloro-1H-pyrrolo[3,2-b]pyridine

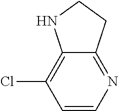

Example 619A (1.0 eq.) was dissolved in POCl₃ (7.0 eq.). The mixture was stirred at 90° C. under N₂ atmosphere until no further conversion was observed. The mixture was allowed to cool to rt. It was quenched with water and extracted with EtOAc. The combined organic layers were dried over Na₂SO₄, filtered and the filtrate was concentrated under reduced pressure. The crude product was purified via flash chromatography using heptane and EtOAc as eluents to obtain Example 619B. ¹H NMR (400 MHz, DMSO-d₆) δ ppm: 11.86 (br s, 1H), 8.27 (d, 1H), 7.72 (d, 1H), 7.25 (d, 1H), 6.66 (d, 1H).

### Example 619C 7-chloro-1-methyl-1H-pyrrolo[3,2-b]pyridine

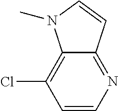

Example 619B (1 eq.) was dissolved in DMF (5 mL/mmol), then cooled to 0° C. To the stirred solution NaH (1.05 eq. 60% suspension in mineral oil) was added under N₂ atmosphere. After gas evolution stopped, Mel (1.05 eq.) was added. The mixture was allowed to warm to rt. The mixture was stirred at rt until no further conversion was observed. Water was added to the mixture, then it was concentrated under reduced pressure. Then water and brine were added and it was extracted with EtOAc. The combined organic layers were dried over Na₂SO₄, filtered and the filtrate was concentrated under reduced pressure. The crude product was purified via flash chromatography using heptane and EtOAc as eluents to obtain Example 619C. ¹H NMR (400 MHz, DMSO-d₆) δ ppm: 8.23 (d, 1H), 7.69 (d, 1H), 7.21 (d, 1H), 6.61 (d, 1H), 4.09 (s, 3H).

### Example 619D methyl (1r,2′S,4S)-4-(3-chloroanilino)-2′-{(2R)-2-methyl-3-[(1-methyl-1H-pyrrolo[3,2-b]pyridin-7-yl)oxy]propyl}-2′,3′-dihydrospiro[cyclohexane-1,1′-indene]-4-carboxylate

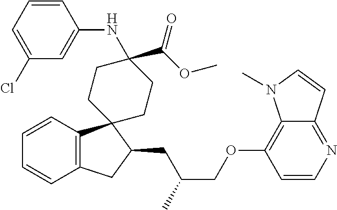 [SMILES: COC(=O)[C@]1(Nc2cccc(Cl)c2)CC[C@@]2(CC1)c1ccccc1C[C@@H]2C[C@@H](C)COc1ccnc2ccn(C)c12]

Preparation 10b1 (1 eq.) was dissolved in toluene (12.5 mL/mmol), then Josiphos SL-J009 PDG3 (0.2 eq.), Example 619C (2 eq.) and Cs₂CO₃ (3 eq.) were added. The mixture was stirred at 110° C. until no further conversion was observed. The mixture was allowed to cool to rt. The solvent was removed under reduced pressure. The crude intermediate was purified via prep RP-HPLC using 25 mM aq. NH₄HCO₃ solution and MeCN as eluents to obtain Example 619D. ¹H NMR (500 MHz, DMSO-d₆) δ ppm: 8.12 (d, 1H), 7.42-7.11 (m, 4H), 7.37 (d, 1H), 7.05 (t, 1H), 6.68 (d, 1H), 6.60 (t, 1H), 6.56 (dm, 1H), 6.44 (dm, 1H), 6.42 (d, 1H), 6.33 (s, 1H), 4.04/4.01 (dd+dd, 2H), 3.90 (s, 3H), 3.64 (s, 3H), 3.02/2.55 (dd+dd, 2H), 2.51-1.32 (m, 8H), 2.17 (m, 1H), 2.09 (m, 1H), 1.53/1.39 (m+m, 2H), 1.09 (d, 3H). HRMS calculated for C₃₄H₃₈N₃O₃Cl: 571.2606; found: 572.2656 (M+H).

### Example 619 (1r,2′S,4S)-4-(3-chloroanilino)-2′-{(2R)-2-methyl-3-[(1-methyl-1H-pyrrolo[3,2-b]pyridin-7-yl)oxy]propyl}-2′,3′-dihydrospiro[cyclohexane-1,1′-indene]-4-carboxylic acid

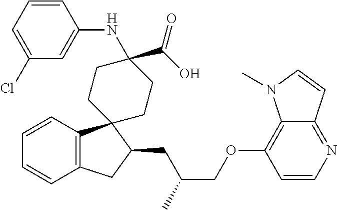 [SMILES: C[C@@H](COc1ccnc2ccn(C)c12)C[C@H]1Cc2ccccc2[C@]12CC[C@@](Nc1cccc(Cl)c1)(C(=O)O)CC2]

Using General procedure 33a and Example 619D as the appropriate ester Example 619 was obtained. ¹H NMR (500 MHz, DMSO-d₆) δ ppm: 8.16 (d, 1H), 7.43 (d, 1H), 7.40-7.11 (m, 4H), 7.04 (t, 1H), 6.74 (d, 1H), 6.62 (t, 1H), 6.54 (dm, 1H), 6.53 (dm, 1H), 6.45 (d, 1H), 6.23 (br s, 1H), 4.05 (m, 2H), 3.91 (s, 3H), 3.02/2.55 (dd+dd, 2H), 2.50-1.36 (m, 8H), 2.17 (m, 1H), 2.09 (m, 1H), 1.52/1.39 (m+m, 2H), 1.09 (d, 3H). HRMS calculated for C₃₃H₃₆N₃O₃Cl: 557.2445; found: 558.2507 (M+H).

### Example 620 and Example 621

### Example 620A rac-(4bR,7aR)-4b,5,6,7,7a,8-hexahydropentaleno[2,1-b]pyridine

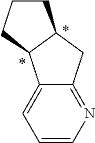 [SMILES: c1cnc2c(c1)[C@H]1CCC[C@H]1C2]

Hexahydropentalen-2 (1H)-one (0.6 g, 4.83 mmol, 1 eq) and prop-2-yn-1-amine (0.53 g, 0.67 mL, 9.66 mmol, 2 eq) were combined in EtOH (10 mL) followed by the addition of NaAuCl₄×2H₂O (58 mg, 0.15 mmol, 0.03 eq) and the mixture was heated at 80° C. in a sealed tube for 24 h. The mixture was allowed to cool to rt, diluted with DCM and filtered through celite. The filtrate was concentrated in vacuo and purified by automated flash chromatography (Combiflash Rf, Silica 24 g, RediSep™ cartridge) eluting with a gradient of 0-40% EtOAc in heptane to afford a racemate, Example 620A, isolated as a pale yellow oil (0.22 g, 1.36 mmol, 28%). LRMS calculated for C₁₁H₁₃N: 159; found: 160 (M+H). ¹H NMR (400 MHz, CDCl₃) δ ppm: 8.36-8.30 (m, 1H), 7.45-7.39 (m, 1H), 7.06-7.00 (m, 1H), 3.65-3.58 (m, 1H), 3.30 (dd, J=17.5, 9.4 Hz, 1H), 2.93-2.82 (m, 1H), 2.77 (dd, J=17.5, 3.3 Hz, 1H), 2.12-2.00 (m, 1H), 1.95-1.84 (m, 1H), 1.71-1.35 (m, 4H).

### Example 620B rac-(4bR,7aR)-1-oxo-4b,5,6,7,7a,8-hexahydro-1λ⁵-pentaleno[2,1-b]pyridine

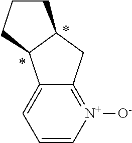 [SMILES: [O-][n+]1cccc2c1C[C@@H]1CCC[C@H]21]

mCPBA (77% purity, 341 mg, 1.52 mmol, 1.1 eq) was added in portions to a solution of Example 620A (220 mg, 1.38 mmol, 1 eq) in DCM (6 mL) and the mixture was stirred at rt for 2 h. 2 M aq. NaOH solution (5 mL) was added and the mixture stirred rapidly for 1 h. The organic phase was separated, and the aq. phase was extracted with DCM. The combined organic extracts were washed with brine, dried (MgSO₄) and concentrated in vacuo to afford a racemate, Example 620B, isolated as an oil that slowly solidified to an off-white solid (220 mg, 1.26 mmol, 91%). LRMS calculated for C₁₁H₁₃NO: 175; found: 176 (M+H). ¹H NMR (400 MHz, CDCl₃) δ ppm: 8.14-8.03 (m, 1H), 7.15-7.05 (m, 2H), 3.75-3.65 (m, 1H), 3.45-3.32 (m, 1H), 3.02-2.86 (m, 2H), 2.12-1.54 (m, 4H), 1.54-1.38 (m, 2H).

### Example 620C rac-(4bR,7aR)-4-chloro-4b,5,6,7,7a,8-hexahydropentaleno[2,1-b]pyridine

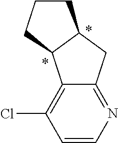 [SMILES: Clc1ccnc2c1[C@H]1CCC[C@H]1C2]

POCl₃ (1.65 mL, 2.71 g, 17.7 mmol, 10 eq) was added dropwise with rapid stirring to cooled Example 620B (310 mg, 1.77 mmol, 1 eq) under N₂ and then the mixture was heated at 80° C. for 6 h. The mixture was allowed to cool to rt, then cooled to −5° C. and quenched by the dropwise addition of water with rapid stirring. The mixture was basified by the careful addition of aq. NH₃ solution and then extracted with DCM. The organic phase was dried (MgSO₄) and concentrated in vacuo. Purification by automated flash chromatography (Combiflash Rf, Silica 24 g, RediSep™ cartridge) eluting with a gradient of 0-30% EtOAc in heptane afforded a racemate, Example 620C, isolated as a colourless oil (195 mg, 1.01 mmol, 57%). LRMS calculated for C₁₁H₁₂ClN: 193; found: 194 (M+H). ¹H NMR (400 MHz, CDCl₃) δ ppm: 8.24 (dd, J=5.4, 0.8 Hz, 1H), 7.06 (dt, J=5.3, 0.8 Hz, 1H), 3.76-3.68 (m, 1H), 3.33 (ddd, J=17.8, 9.8, 0.8 Hz, 1H), 2.98-2.87 (m, 1H), 2.86-2.78 (m, 1H), 2.21-2.10 (m, 1H), 1.99-1.89 (m, 1H), 1.78-1.68 (m, 1H), 1.64-1.40 (m, 3H).

### Example 620 (1r,2′S,4S)-4-(3-chloroanilino)-2′-[(2R)-3-{[(4bR*,7aR*)-4b,5,6,7,7a,8-hexahydropentaleno[2,1-b]pyridin-4-yl]oxy}-2-methylpropyl]-2′,3′-dihydrospiro[cyclohexane-1,1′-indene]-4-carboxylic acid, diastereoisomer 2

and

### Example 621 (1r,2′S,4S)-4-(3-chloroanilino)-2′-[(2R)-3-{[(4bR*,7aR*)-4b,5,6,7,7a,8-hexahydropentaleno[2,1-b]pyridin-4-yl]oxy}-2-methylpropyl]-2′,3′-dihydrospiro[cyclohexane-1,1′-indene]-4-carboxylic acid, diastereoisomer 1

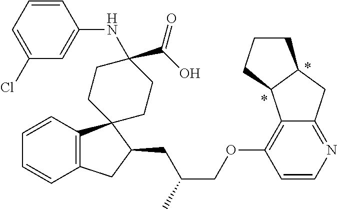 [SMILES: C[C@@H](COc1ccnc2c1[C@H]1CCC[C@H]1C2)C[C@H]1Cc2ccccc2[C@]12CC[C@@](Nc1cccc(Cl)c1)(C(=O)O)CC2]

Using General procedure 31c and Preparation 10b1 as the appropriate indane and Example 620C as the appropriate aryl chloride a mixture of diastereoisomers was obtained. They were separated by chiral chromatography. Column: AD, 50 mm×500 mm, 20 μm, Eluents: 50:50 EtOH/heptane. The diastereoisomer eluting later was hydrolyzed according to General procedure 33a to obtain Example 620. HRMS calculated for C₃₆H₄₁N₂O₃Cl: 584.2806; found: 585.2865 (M+H).

The diastereoisomer eluting earlier was hydrolyzed according to General procedure 33a to obtain Example 621. HRMS calculated for C₃₆H₄₁N₂O₃Cl: 584.2806; found: 585.2874 (M+H).

### Example 622 and Example 623

### Example 622A 2-(4-methoxyphenyl)-5,5-dimethyl-1,3-dioxane

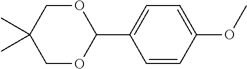

1-(dimethoxymethyl)-4-methoxy-benzene (10.0 g, 55.0 mmol) was dissolved in dry DCM (330 mL). 2,2-dimethylpropane-1,3-diol (4.81 g, 46.2 mmol) and PPTS (1.38 g, 5.5 mmol) were added to the mixture and stirred at rt for 3 h. NaHCO₃ (924 mg, 11.0 mmol) was added to the mixture and stirred at rt for 30 min. The reaction mixture was concentrated under reduced pressure. The crude product was purified via flash chromatography using heptane and EtOAc as eluents to obtain Example 622A. ¹H NMR (400 MHz, DMSO-d₆) δ ppm: 7.34 (m, 2H), 6.90 (m, 2H), 5.34 (s, 1H), 3.74 (s, 3H), 3.65/3.59 (d+d, 4H), 1.18 (s, 3H), 0.74 (s, 3H).

### Example 622B 3-[(4-methoxyphenyl)methoxy]-2,2-dimethylpropan-1-ol

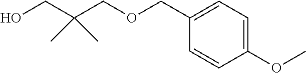 [SMILES: COc1ccc(COCC(C)(C)CO)cc1]

Example 622A (9.85 g, 44.3 mmol) was dissolved in dry DCM (220 mL) and cooled to 0° C. DIBAL-H 1 M in DCM (111 mL) was added dropwise at 0° C. The reaction mixture was allowed to warm to rt and stirred until no further conversion was observed. The mixture was cooled to 0° C., MeOH (42 mL) was added dropwise at 0° C., then water (42 mL) and DCM (100 mL) was added. The mixture was stirred at rt for 1 h, then it was diluted with water (40 mL). The layers were separated. The aq. layer was filtered and extracted with DCM. The combined organic layers were dried over Na₂SO₄, filtered and the filtrate was concentrated under reduced pressure to obtain Example 622B. ¹H NMR (400 MHz, DMSO-d₆) δ ppm: 7.23 (m, 2H), 6.90 (m, 2H), 4.42 (t, 1H), 4.36 (s, 2H), 3.74 (s, 3H), 3.16 (d, 2H), 3.12 (s, 2H), 0.79 (s, 6H).

### Example 622C 1-[(3-bromo-2,2-dimethylpropoxy)methyl]-4-methoxybenzene

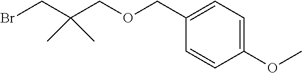 [SMILES: COc1ccc(COCC(C)(C)CBr)cc1]

Using General procedure 3 and Example 622B as the appropriate alcohol Example 622C was obtained. ¹H NMR (500 MHz, DMSO-d₆) δ ppm: 7.24 (dm, 2H), 6.91 (dm, 2H), 4.40 (s, 2H), 3.74 (s, 3H), 3.46 (s, 2H), 3.20 (s, 2H), 0.96 (s, 6H). HRMS calculated for C₁₃H₁₉BrO₂: 286.0569; found 286.05564 (M+).

### Example 622D bromido{3-[(4-methoxyphenyl)methoxy]-2,2-dimethylpropyl}zinc

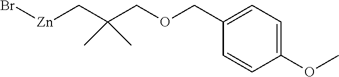

Using General procedure 4 and Example 622C as the appropriate bromo compound Example 622D was obtained.

### Example 622E methyl (1r,4r)-4-(3-chloroanilino)-2′-{3-[(4-methoxyphenyl)methoxy]-2,2-dimethylpropyl}spiro[cyclohexane-1,1′-indene]-4-carboxylate

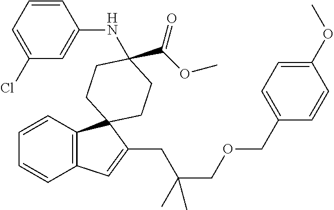

Using General procedure 27a and Preparation 6c as the appropriate 2-bromo-indene derivative and Example 622D as the appropriate Zn reagent Example 622E was obtained. ¹H NMR (500 MHz, DMSO-d₆) δ ppm: 7.69-7.04 (m, 4H), 7.23 (m, 2H), 7.09 (t, 1H), 6.87 (m, 2H), 6.65 (t, 1H), 6.60 (dm, 1H), 6.48 (dm, 1H), 6.42 (s, 1H), 6.40 (s, 1H), 4.40 (s, 2H), 3.73 (s, 3H), 3.69 (s, 3H), 3.19 (s, 2H), 2.37/2.20 (m+m, 4H), 2.22 (s, 2H), 2.21/0.80 (m+m, 4H), 0.95 (s, 6H). HRMS calculated for C₃₅H₄₀ClNO₄: 573.2646; found 574.2718 (M+H).

### Example 622F methyl (1r,4r)-4-(3-chloroanilino)-2′-(3-hydroxy-2,2-dimethylpropyl)spiro[cyclohexane-1,1′-indene]-4-carboxylate

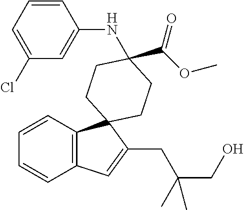

Using General procedure 28b and Example 622E as the appropriate PMB derivative Example 622F was obtained. ¹H NMR (500 MHz, DMSO-d₆) δ ppm: 7.68-7.05 (m, 4H), 7.10 (t, 1H), 6.64 (t, 1H), 6.60 (dm, 1H), 6.58 (s, 1H), 6.49 (dm, 1H), 6.42 (s, 1H), 4.64 (t, 1H), 3.69 (s, 3H), 3.24 (d, 2H), 2.37/2.20 (m+m, 4H), 2.18 (s, 2H), 2.13/0.82 (m+m, 4H), 0.91 (s, 6H). HRMS calculated for C₂₇H₃₂ClNO₃: 453.2071; found 454.2147 (M+H).

### Example 622G methyl (1r,4r)-4-(3-chloroanilino)-2′-(3-hydroxy-2,2-dimethylpropyl)-2′,3′-dihydrospiro[cyclohexane-1,1′-indene]-4-carboxylate, enantiomer 1

and

### Example 622H methyl (1r,4r)-4-(3-chloroanilino)-2′-(3-hydroxy-2,2-dimethylpropyl)-2′,3′-dihydrospiro[cyclohexane-1,1′-indene]-4-carboxylate, enantiomer 2

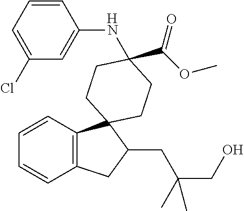

Using General procedure 19 and Example 622F as the appropriate indene, a racemate was obtained. The enantiomers were separated by chiral chromatography. Column: AS-V, 100 mm×500 mm, 20 μm, Eluents: 10:90 EtOH/heptane. The enantiomer eluting earlier was collected as Example 622G. ¹H NMR (500 MHz, DMSO-d₆) δ ppm: 7.50-7.10 (m, 4H), 7.07 (t, 1H), 6.59 (t, 1H), 6.57 (dm, 1H), 6.44 (dm, 1H), 6.29 (s, 1H), 4.48 (t, 1H), 3.64 (s, 3H), 3.15/3.13 (dd+dd, 2H), 2.97/2.57 (dd+dd, 2H), 2.51-1.02 (m, 8H), 1.89 (m, 1H), 1.42/1.24 (m, 2H), 0.85 (s, 6H). HRMS calculated for C₂₇H₃₄ClNO₃: 455.2227; found 456.2301 (M+H).

The enantiomer eluting later was collected as Example 622H. HRMS calculated for C₂₇H₃₄ClNO₃: 455.2227; found 546.2296 (M+H).

### Example 622 (1r,4r)-4-(3-chloroanilino)-2′-{2,2-dimethyl-3-[(thieno[2,3-b]pyridin-4-yl)oxy]propyl}-2′,3′-dihydrospiro[cyclohexane-1,1′-indene]-4-carboxylic acid, enantiomer 1

and

### Example 623 (1r,4r)-4-(3-chloroanilino)-2′-{2,2-dimethyl-3-[(thieno[2,3-b]pyridin-4-yl)oxy]propyl}-2′,3′-dihydrospiro[cyclohexane-1,1′-indene]-4-carboxylic acid, enantiomer 2

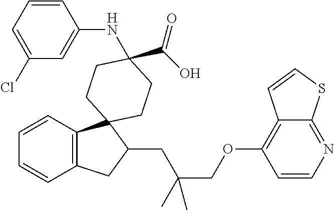

Using General procedure 32 and Example 622G as the appropriate indane and thieno[2,3-b]pyridin-4-ol as the appropriate alcohol Example 622 was obtained. ¹H NMR (500 MHz, DMSO-d₆) δ ppm: 12.61 (br s, 1H), 8.51 (d, 1H), 7.87 (d, 1H), 7.50-7.09 (m, 4H), 7.48 (d, 1H), 7.07 (t, 1H), 7.00 (d, 1H), 6.62 (t, 1H), 6.57 (dm, 1H), 6.54 (dm, 1H), 6.19 (br s, 1H), 4.04/4.01 (d+d, 2H), 3.00/2.59 (dd+dd, 2H), 2.49-1.06 (m, 8H), 1.98 (m, 1H), 1.74/1.47 (d+dd, 2H), 1.11 (s, 3H), 1.09 (s, 3H). HRMS calculated for C₃₃H₃₅N₂O₃SCl: 574.2057; found: 575.2118 (M+H).

Using General procedure 32 and Example 622H as the appropriate indane and thieno[2,3-b]pyridin-4-ol as the appropriate alcohol Example 623 was obtained. HRMS calculated for C₃₃H₃₅N₂O₃SCl: 574.2057; found: 575.2124 (M+H).

### Example 624

### Example 624A 4-chloro-5-ethyl-1-oxo-6,7-dihydro-5H-1λ⁵-cyclopenta[b]pyridine

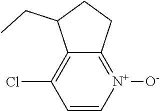

To a solution of Example 612D (465 mg, 2.56 mmol, 1 eq) in DCM (10 mL) cooled to 0° C. was added mCPBA (70% purity, 757 mg, 3.07 mmol, 1.2 eq) and the mixture was stirred at rt for 3 h. The mixture was partitioned between DCM and 1 M aq. NaOH solution and the organic phase was washed with brine, dried (MgSO₄) and concentrated in vacuo to afford a racemate, Example 624A, isolated as a yellow oil (506 mg, 2.56 mmol, 100%). LRMS calculated for C₁₀H₁₂ClNO: 197; found: 198 (M+H).

### Example 624B 4-chloro-5-ethyl-6,7-dihydro-5H-cyclopenta[b]pyridin-7-ol

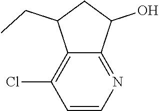

To a solution of Example 624A (520 mg, 2.63 mmol, 1 eq) in DCM (10 mL), cooled to 0° C., was added TFAA (1.1 mL, 7.89 mmol, 3 eq) and the mixture was stirred at rt for 5 h. The mixture was quenched with 2 M aq. NaOH solution (15 mL) and stirred vigorously for 1 h, then partitioned between water and a solution of 25% iPrOH in DCM and the organic phase separated. The aq. phase was extracted with 25% iPrOH in DCM and the combined organic extracts were dried (MgOS₄) and concentrated in vacuo. Purification by automated flash chromatography (CombiFlash Rf, 12 g RediSep™ silica cartridge) eluting with a gradient of 20-70% EtOAc in heptane afforded a racemic mixture of diastereoisomers, Example 624B, isolated as a colourless glass (188 mg, 0.95 mmol, 36%). LRMS calculated for C₁₀H₁₂ClNO: 197; found: 198 (M+H).

### Example 624C 4-chloro-5-ethyl-7-[(4-methoxyphenyl)methoxy]-6,7-dihydro-5H-cyclopenta[b]pyridine

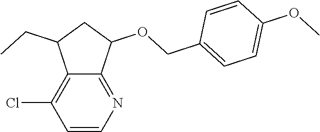 [SMILES: CCC1CC(OCc2ccc(OC)cc2)c2nccc(Cl)c21]

To an oven-dried flask was added Example 624B (188 mg, 0.95 mmol, 1 eq), PMB-Cl (154 μL, 1.14 mmol, 1.2 eq) and TBAI (35 mg, 95.1 μmol, 0.1 eq) in DMF (4 mL). NaH (60% dispersion; 76 mg, 1.9 mmol, 2 eq) was added in portions and the mixture was allowed to stir at rt for 18 h. The mixture was partitioned between DCM and sat. aq. NaHCO₃ solution. The phases were separated, and the organic phase was washed with brine, dried (PTFE phase separator) and concentrated in vacuo. Purification by automated flash chromatography (CombiFlash Rf, 4 g RediSep™ silica cartridge) eluting with a gradient of 0-20% EtOAc in heptane afforded a racemic mixture of diastereoisomers, Example 624C, isolated as a colourless oil (241 mg, 0.76 mmol, 80%). LRMS calculated for C₁₈H₂₀ClNO₂: 317; found: 318 (M+H).

### Example 624D (1r,2′S,4S)-4-(3-chloroanilino)-2′-[(2R)-3-({5-ethyl-7-[(4-methoxyphenyl)methoxy]-6,7-dihydro-5H-cyclopenta[b]pyridin-4-yl}oxy)-2-methylpropyl]-2′,3′-dihydrospiro[cyclohexane-1,1′-indene]-4-carboxylic acid

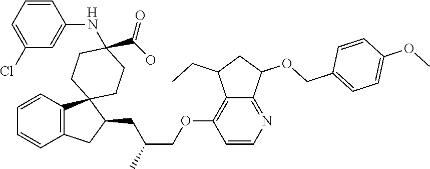

Using General Procedure 31c and Example 611B as the appropriate alcohol and Example 624C as the appropriate aryl chloride, a mixture of 4 diastereoisomers, Example 624D, was isolated as a beige glass. LRMS calculated for C₄₃H₄₉ClN₂O₅: 708; found: 709 (M+H).

### Example 624 (1r,2′S,4S)-4-(3-chloroanilino)-2′-{(2R)-3-[(5-ethyl-7-hydroxy-6,7-dihydro-5H-cyclopenta[b]pyridin-4-yl)oxy]-2-methylpropyl}-2′,3′-dihydrospiro[cyclohexane-1,1′-indene]-4-carboxylic acid

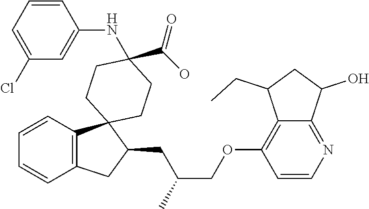

Using General Procedure 28b and Example 624D as the appropriate PMB derivative, a mixture of 4 diastereoisomers, Example 624 was isolated as an off-white powder. LRMS calculated for C₃₅H₄₁ClN₂O₄: 588; found: 589 (M+H). ¹H NMR (400 MHz, DMSO-d₆) δ ppm: 8.27 (d, J=5.7 Hz, 1H), 7.42-7.34 (m, 1H), 7.24-7.10 (m, 3H), 7.07-6.99 (m, 1H), 6.85 (d, J=5.7 Hz, 1H), 6.66-6.60 (m, 1H), 6.58-6.49 (m, 2H), 5.35-5.18 (m, 1H), 4.98-4.75 (m, 1H), 4.00-3.84 (m, 2H), 3.24-2.86 (m, 2H), 2.60-1.22 (m, 17H), 1.08-0.99 (m, 3H), 0.83-0.62 (m, 3H).

### Example 625 (1r,2′S,4S)-2′-{(2R)-3-[(1,3-benzothiazol-7-yl)oxy]-2-methylpropyl}-4-(3-chloroanilino)-2′,3′-dihydrospiro[cyclohexane-1,1′-indene]-4-carboxylic acid

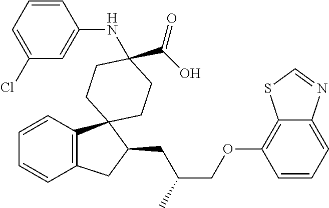

Using General procedure 32 and Preparation 10b1 as the appropriate indane and 1,3-benzothiazol-7-ol as the appropriate alcohol Example 625 was obtained. ¹H NMR (500 MHz, DMSO-d₆) δ ppm: 9.32 (s, 1H), 7.68 (d, 1H), 7.47 (t, 1H), 7.37 (d, 1H), 7.18 (d, 1H), 7.14/7.14 (t+t, 2H), 7.07 (d, 1H), 7.01 (t, 1H), 6.64 (t, 1H), 6.55 (dd, 1H), 6.50 (dd, 1H), 6.16 (br s, 1H), 4.08/4.03 (dd+dd, 2H), 3.00/2.55 (dd+dd, 2H), 2.44-1.41 (m, 8H), 2.16 (m, 1H), 2.05 (m, 1H), 1.46/1.38 (m+m, 2H), 1.07 (d, 3H). HRMS calculated for C₃₂H₃₃N₂O₃SCl: 560.1901; found: 561.1958 (M+H).

### Example 626

### Example 626A tert-butyl 4-hydroxy-1H-pyrrolo[2,3-b]pyridine-1-carboxylate

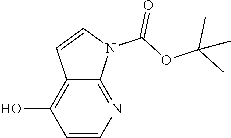 [SMILES: CC(C)(C)OC(=O)n1ccc2c(O)ccnc21]

1H-pyrrolo[2,3-b]pyridin-4-ol (1.0 eq.) and DMAP (0.05 eq.) were dissolved in the mixture of DCM (4.8 mL/mmol) and DMF (4.0 mL/mmol). The solution was cooled to 0° C., then Boc₂0 (1.1 eq.) was added at 0° C. The mixture was allowed to warm to rt, and was stirred at rt until no further conversion was observed. The mixture was concentrated under reduced pressure. The crude product was purified via flash chromatography using heptane and EtOAc, then EtOAc and MeOH as eluents to obtain Example 626A. ¹H NMR (500 MHz, DMSO-d₆) δ ppm: 11.58 (br s, 1H), 7.97 (d, 1H), 6.84 (t, 1H), 6.41 (dd, 1H), 5.98 (d, 1H), 1.63 (s, 9H). HRMS calculated for C₁₂H₁₄N₂O₃: 234.1004; found: 179.0454 (M-tBu+H).

### Example 626 (1r,2′S,4S)-4-(3-chloroanilino)-2′-{(2R)-2-methyl-3-[(1H-pyrrolo[2,3-b]pyridin-4-yl)oxy]propyl}-2′,3′-dihydrospiro[cyclohexane-1,1′-indene]-4-carboxylic acid

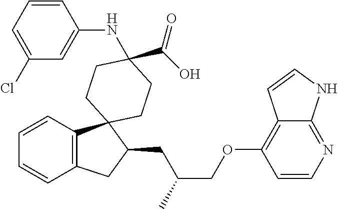

Using General procedure 32 and Preparation 10b1 as the appropriate indane and Example 626A as the appropriate alcohol Example 626 was obtained. ¹H NMR (400 MHz, DMSO-d₆) δ ppm: 11.51 (s, 1H), 8.04 (d, 1H), 7.38 (dm, 1H), 7.23 (dd, 1H), 7.21 (dm, 1H), 7.15-7.14 (m, 2H), 7.03 (t, 1H), 6.63 (t, 1H), 6.60 (d, 1H), 6.55 (dd, 1H), 6.52 (dd, 1H), 6.37 (dd, 1H), 6.22 (br s, 1H), 4.05/3.99 (dd+dd, 2H), 3.00/2.56 (dd+dd, 2H), 2.44-1.39 (m, 8H), 2.15 (m, 1H), 2.06 (m, 1H), 1.48/1.38 (m+m, 2H), 1.07 (d, 3H). HRMS calculated for C₃₂H₃₄N₃O₃Cl: 543.2289; found: 544.2352 (M+H).

### Example 627

### Example 627A tert-butyl thieno[3,2-b]pyridin-7-ylcarbamate

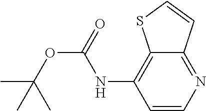 [SMILES: CC(C)(C)OC(=O)Nc1ccnc2ccsc12]

Boc₂O (1.0 eq.) was dissolved in THE (2.5 mL/mmol), then thieno[3,2-b]pyridin-7-amine (1.0 eq.) was added. The mixture was stirred at rt until no further conversion was observed. The mixture was concentrated under reduced pressure. To the residue brine was added, then it was extracted with DCM. The combined organic layers were dried over Na₂SO₄, filtered and the filtrate was concentrated under reduced pressure. The crude product was purified via flash chromatography using DCM and MeOH as eluents to obtain Example 627A. ¹H NMR (400 MHz, DMSO-d₆) δ ppm: 9.94 (s, 1H), 8.50 (d, 1H), 8.05 (d, 1H), 7.60 (d, 1H), 7.48 (d, 1H), 1.52 (s, 9H).

### Example 627 (1r,2′S,4S)-4-(3-chloroanilino)-2′-{(2R)-2-methyl-3-[(thieno[3,2-b]pyridin-7-yl)amino]propyl}-2′,3′-dihydrospiro[cyclohexane-1,1′-indene]-4-carboxylic acid

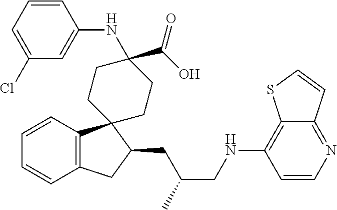 [SMILES: C[C@@H](CNc1ccnc2ccsc12)C[C@H]1Cc2ccccc2[C@]12CC[C@@](Nc1cccc(Cl)c1)(C(=O)O)CC2]

Using General procedure 32 and Preparation 10b1 as the appropriate indane and Example 627A as the appropriate amine, an intermediate was obtained which was dissolved in DCM (12 mL/mmol), then the solution was cooled to 0° C., then TFA (26.0 eq.) was added. The mixture was allowed to warm to rt, and was stirred at rt until no further conversion was observed. TEA (16.5 eq.) was added to the mixture, then it was concentrated under reduced pressure. The crude product was purified via prep RP-HPLC using 25 mM aq. NH₄HCO₃ solution and MeCN as eluents to obtain Example 627. ¹H NMR (400 MHz, DMSO-d₆) δ ppm: 8.19 (d, 1H), 7.87 (d, 1H), 7.39-7.09 (m, 4H), 7.34 (d, 1H), 7.07 (t, 1H), 6.83 (t, 1H), 6.64 (t, 1H), 6.55 (m, 2H), 6.44 (d, 1H), 6.24 (br s, 1H), 3.17/3.12 (dd+dd, 2H), 2.90/2.44 (dd+dd, 2H), 2.48-1.32 (m, 8H), 2.10 (m, 1H), 1.90 (m, 1H), 1.51/1.42 (m+m, 2H), 0.96 (d, 3H). HRMS calculated for C₃₂H₃₄N₃O₂SCl: 559.2061; found: 560.2132 (M+H).

### Example 628

### Example 628A tert-butyl 3-(acetyloxy)-1H-indole-1-carboxylate

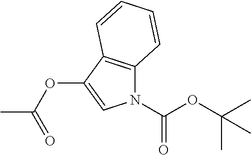 [SMILES: CC(=O)Oc1cn(C(=O)OC(C)(C)C)c2ccccc12]

1H-indol-3-yl acetate (1.0 eq.) and DMAP (0.05 eq.) were dissolved in dry DCM (5 mL/mmol) under N₂ atmosphere. The solution was cooled to 0° C., then Boc₂0 (1.1 eq.) was added. The reaction mixture was allowed to warm to rt while stirring. The mixture was stirred at rt under N₂ atmosphere until no further conversion was observed. The mixture was concentrated under reduced pressure. The crude product was purified via flash chromatography using heptane and EtOAc as eluents to obtain Example 628A. ¹H NMR (400 MHz, DMSO-d₆) δ ppm: 8.09 (d, 1H), 7.70 (s, 1H), 7.54 (dt, 1H), 7.40 (td, 1H), 7.29 (td, 1H), 2.36 (s, 3H), 1.62 (s, 9H).

### Example 628B tert-butyl 3-oxo-2,3-dihydro-1H-indole-1-carboxylate

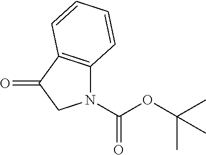 [SMILES: CC(C)(C)OC(=O)N1CC(=O)c2ccccc21]

Example 628A (1.0 eq.) was dissolved in MeOH (2 mL/mmol), then the solution of NaOMe (0.1 eq.) in MeOH (5 mL/mmol NaOMe) was added. The mixture was stirred at rt until no further conversion was observed. The mixture was concentrated under reduced pressure. The crude product was purified via flash chromatography using heptane and EtOAc as eluents to obtain Example 628B. ¹H NMR (400 MHz, DMSO-d₆) δ ppm: 8.12 (m, 1H), 7.72 (tm, 1H), 7.65 (dm, 1H), 7.19 (tm, 1H), 4.27 (s, 2H), 1.53 (s, 9H). HRMS calculated for C₁₃H₁₅NO₃: 233.1052; found: 234.1129 (M+H).

### Example 628 (1r,2′S,4S)-4-(3-chloroanilino)-2′-{(2R)-3-[(1H-indol-3-yl)oxy]-2-methylpropyl}-2′,3′-dihydrospiro[cyclohexane-1,1′-indene]-4-carboxylic acid

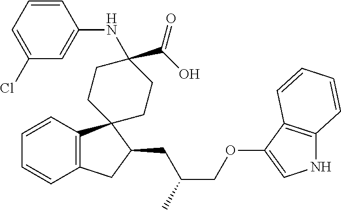 [SMILES: C[C@@H](COc1c[nH]c2ccccc12)C[C@H]1Cc2ccccc2[C@]12CC[C@@](Nc1cccc(Cl)c1)(C(=O)O)CC2]

Using General procedure 32 and Preparation 10b1 as the appropriate indane and Example 628B as the appropriate alcohol Example 628 was obtained. ¹H NMR (400 MHz, DMSO-d₆) δ ppm: 10.37 (d, 1H), 7.42 (d, 1H), 7.40-7.10 (m, 4H), 7.25 (d, 1H), 7.05 (t, 1H), 7.02 (t, 1H), 6.90 (t, 1H), 6.89 (d, 1H), 6.63 (t, 1H), 6.56 (dd, 1H), 6.51 (dd, 1H), 6.22 (br s, 1H), 3.80 (m, 2H), 2.99/2.56 (dd+dd, 2H), 2.50-1.36 (m, 8H), 2.15 (m, 1H), 2.01 (m, 1H), 1.49/1.33 (m+m, 2H), 1.05 (d, 3H). HRMS calculated for C₃₃H₃₅N₂O₃Cl: 542.2336; found: 543.2413 (M+H).

### Example 629 and Example 630

### Example 629A 5-methylidene-6,7-dihydro-5H-cyclopenta[b]pyridine

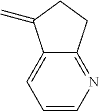

To a suspension of methyltriphenylphosphonium bromide (17.44 g, 48.82 mmol, 1.3 eq) in THE (100 mL), cooled to 0° C. under N₂, was added nBuLi (20.51 mL, 2.38 M in hexanes, 48.82 mmol, 1.3 eq) dropwise and then the mixture was stirred at 0° C. for 40 min. 6,7-dihydro-5H-cyclopenta[b]pyridin-5-one (5 g, 37.55 mmol, 1 eq) in THE (20 mL) was added dropwise at 0° C., and the mixture was allowed to warm to rt and stirred for 18 h. The mixture was cooled to 0° C. and quenched by the careful addition of sat. aq. NaHCO₃ solution. Excess THE was removed in vacuo, and the aq. phase was extracted with EtOAc. The organic phase was dried (MgSO₄) and concentrated in vacuo. Purification by automated flash chromatography (Combiflash Rf, Silica 80 g, RediSep™ cartridge) eluting with a gradient of 0-33% EtOAc (3% TEA) in heptane afforded Example 629A as a colourless oil (2.7 g, 20.58 mmol, 55%). LRMS calculated for C₉H₉N: 131; found: 132 (M+H). ¹H NMR (400 MHz, DMSO-d₆) δ ppm: 8.40 (dd, J=4.8, 1.6 Hz, 1H), 7.94 (dd, J=7.8, 1.6 Hz, 1H), 7.25-7.19 (m, 1H), 5.63 (t, J=2.6 Hz, 1H), 5.15 (t, J=2.2 Hz, 1H), 3.03-2.96 (m, 2H), 2.81-2.74 (m, 2H).

### Example 629B 5-methyl-6,7-dihydro-5H-cyclopenta[b]pyridine

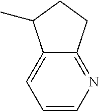

To a solution of Example 629A (2.7 g, 20.58 mmol, 1 eq) in EtOH (80 mL) was added 10% Pd/C (100 mg) under a N₂ atmosphere. The mixture was evacuated and backfilled with N₂ (×3), then evacuated and backfilled with H₂ and shaken at rt under an atmosphere of H₂ for 2 h. The reaction was filtered through celite, washed with EtOH and the solvents removed in vacuo to afford a racemate, Example 629B, isolated as a colourless oil (2.65 g, 19.9 mmol, 97%). LRMS calculated for C₉H₁₁N: 133; found 134 (M+H). ¹H NMR (400 MHz, DMSO-d₆) δ ppm; 8.31-8.26 (m, 1H), 7.61-7.56 (m, 1H), 7.16-7.11 (m, 1H), 3.25-3.13 (m, 1H), 2.95-2.80 (m, 2H), 2.35-2.25 (m, 1H), 1.63-1.51 (m, 1H), 1.25 (d, J=6.8 Hz, 3H).

### Example 629C 5-methyl-1-oxo-6,7-dihydro-5H-1λ⁵-cyclopenta[b]pyridine

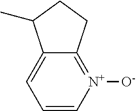

mCPBA (70% purity, 5.89 g, 23.88 mmol, 1.2 eq) was added portionwise to a solution of Example 629B (2.65 g, 19.9 mmol, 1 eq) in DCM (50 mL), cooled to 0° C. under N₂ and stirred at rt for 2.5 h. The mixture was diluted with DCM, 2 M aq. NaOH solution was added, and the mixture was stirred vigorously for 1 h. The layers were separated, and the aq. phase was extracted with 5:1 DCM/IPA. The combined organic extracts were washed with sat. aq. NaHCO₃ solution, dried (MgSO₄) and concentrated in vacuo to afford a racemate, Example 629C, isolated as a white solid (2.45 g, 16.42 mmol, 83%). LRMS calculated for C₉H₁₁NO: 149; found: 150 (M+H). ¹H NMR (400 MHz, DMSO-d₆) δ ppm: 8.06-8.02 (m, 1H), 7.28-7.19 (m, 2H), 3.34-3.23 (m, 1H), 3.04-2.94 (m, 1H), 2.90-2.80 (m, 1H), 2.37-2.27 (m, 1H), 1.68-1.56 (m, 1H), 1.25 (d, J=6.9 Hz, 3H).

### Example 629D 4-chloro-5-methyl-6,7-dihydro-5H-cyclopenta[b]pyridine

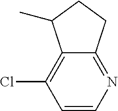

Example 629C (2.45 g, 16.42 mmol, 1 eq) was heated at 90° C. in POCl₃ (15.31 mL, 164.22 mmol, 10 eq) for 72 h. The mixture was allowed to cool to rt and added dropwise to a rapidly stirred mixture of ice/water. The solution was basified to pH 11 with aq. NH₃ solution and then extracted with DCM. The organic phase was dried (MgSO₄) and concentrated in vacuo. Purification by automated flash chromatography (Combiflash Rf, Silica 40 g, RediSep™ cartridge) eluting with a gradient of 0-35% EtOAc (3% TEA) in heptane afforded a racemate, Example 629D, isolated as a yellow oil (1.85 g, 11.04 mmol, 67%). LRMS calculated for C₉H₁₀NCl: 167, found: 168 (M+H). ¹H NMR (400 MHz, DMSO-d₆) δ ppm: 8.27 (d, J=5.4, 1H), 7.28-7.24 (m, 1H), 3.44-3.34 (m, 1H), 3.18-3.07 (m, 1H), 2.88 (ddd, J=16.9, 9.3, 3.4, 1H), 2.35-2.23 (m, 1H), 1.79-1.70 (m, 1H), 1.25 (d, J=7.0 Hz, 3H).

### Example 629E (1r,4R)-4-(3-chloroanilino)-2′-[(2R)-3-hydroxy-2-methylpropyl]spiro[cyclohexane-1,1′-indene]-4-carboxylic acid

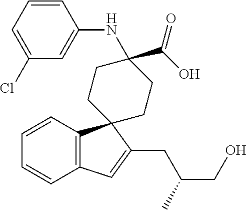 [SMILES: C[C@@H](CO)CC1=Cc2ccccc2[C@]12CC[C@@](Nc1cccc(Cl)c1)(C(=O)O)CC2]

Using General procedure 33b and Preparation 10a (580 mg, 1.32 mmol, 1 eq) as the appropriate ester, Example 629E was obtained as an off-white foam. LRMS calculated for C₂₅H₂₈ClNO₃: 425; found: 426 (M+H). ¹H NMR (400 MHz, DMSO-d₆) δ ppm: 12.80 (br s, 1H), 7.66 (d, J=7.5 Hz, 1H), 7.29 (dd, J=7.4, 1.2 Hz, 1H), 7.26-7.19 (m, 1H), 7.15-7.07 (m, 2H), 6.64 (t, J=2.1 Hz, 1H), 6.61-6.55 (m, 2H), 6.48-6.45 (m, 1H), 6.36 (br s, 1H), 3.39-3.27 (m, 2H), 2.47-2.32 (m, 3H), 2.24-2.04 (m, 4H), 2.04-1.86 (m, 2H), 0.94-0.81 (m, 5H).

### Example 629 (1r,4R)-4-(3-chloroanilino)-2′-{(2R)-2-methyl-3-[(5-methyl-6,7-dihydro-5H-cyclopenta[b]pyridin-4-yl)oxy]propyl}spiro[cyclohexane-1,1′-indene]-4-carboxylic acid, diastereoisomer 2

### Example 630 (1r,4R)-4-(3-chloroanilino)-2′-{(2R)-2-methyl-3-[(5-methyl-6,7-dihydro-5H-cyclopenta[b]pyridin-4-yl)oxy]propyl}spiro[cyclohexane-1,1′-indene]-4-carboxylic acid, diastereoisomer 1

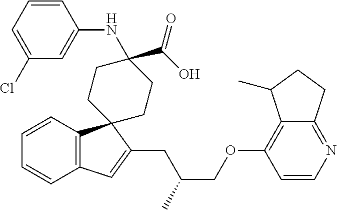 [SMILES: CC1CCc2nccc(OC[C@H](C)CC3=Cc4ccccc4[C@]34CC[C@@](Nc3cccc(Cl)c3)(C(=O)O)CC4)c21]

Using General procedure 31c and Example 629E as the appropriate indene and Example 629D as the appropriate aryl chloride, followed by General procedure 17a a mixture of diastereoisomers was obtained. They were separated via prep RP-HPLC using 25 mM aq. NH₄HCO₃ solution and MeCN as eluents. The diastereoisomer eluting later was hydrolyzed according to General procedure 33a to obtain Example 629. ¹H NMR (500 MHz, DMSO-d₆) δ ppm: 12.87 (br s, 1H), 8.14 (d, 1H), 7.70-7.08 (m, 4H), 7.06 (t, 1H), 6.76 (d, 1H), 6.64 (t, 1H), 6.55 (dm, 1H), 6.54 (t, 1H), 6.53 (dm, 1H), 6.32 (br s, 1H), 4.01/3.97 (dd+dd, 2H), 3.23 (m, 1H), 2.90/2.74 (m+m, 2H), 2.50-0.80 (m, 11H), 2.17/1.54 (m+m, 2H), 1.17 (d, 3H), 1.09 (d, 3H). HRMS calculated for C₃₄H₃₇N₂O₃Cl: 556.2493; found: 557.2563 (M+H).

The diastereoisomer eluting earlier was hydrolyzed according to General procedure 33a to obtain Example 630. ¹H NMR (500 MHz, DMSO-d₆) δ ppm: 12.87 (br s, 1H), 8.11 (d, 1H), 7.69-7.07 (m, 4H), 7.07 (t, 1H), 6.72 (d, 1H), 6.65 (t, 1H), 6.56 (m, 1H), 6.55 (dm, 1H), 6.54 (s, 1H), 6.34 (br s, 1H), 3.98 (m, 2H), 3.09 (m, 1H), 2.86/2.68 (m+m, 2H), 2.47/2.21 (m+m, 2H), 2.45-0.66 (m, 8H), 2.43 (m, 1H), 2.04/1.47 (m+m, 2H), 1.14 (d, 3H), 1.10 (d, 3H). HRMS calculated for C₃₄H₃₇N₂O₃Cl: 556.2493; found: 557.2559 (M+H).

### Example 631

### Example 631A methyl (1r,2′S,4S)-4-(3-chloroanilino)-2′-{(2R)-3-[(3-chloro-1-methyl-1H-pyrrolo[3,2-b]pyridin-7-yl)oxy]-2-methylpropyl}-2′,3′-dihydrospiro[cyclohexane-1,1′-indene]-4-carboxylate

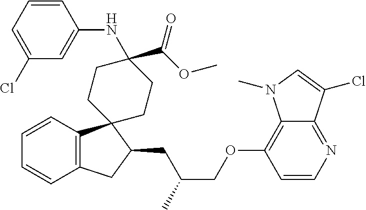 [SMILES: COC(=O)[C@]1(Nc2cccc(Cl)c2)CC[C@@]2(CC1)c1ccccc1C[C@@H]2C[C@@H](C)COc1ccnc2c(Cl)cn(C)c12]

Example 619D (1 eq.) was dissolved in DMF (0.05 M), the solution was cooled to 0° C., then NCS (1.0 eq.) was added. The reaction mixture was allowed to warm to rt and stirred until no further conversion was observed. Then it was diluted with THF and injected on prep RP-HPLC and purified using 25 mM aq. NH₄HCO₃ solution and MeCN as eluents to obtain Example 631A. ¹H NMR (500 MHz, DMSO-d₆) δ ppm: 8.20 (d, 1H), 7.61 (s, 1H), 7.41-7.11 (m, 4H), 7.05 (t, 1H), 6.79 (d, 1H), 6.59 (t, 1H), 6.56 (dm, 1H), 6.44 (dm, 1H), 6.33 (s, 1H), 4.06+4.02 (dd+dd, 2H), 3.88 (s, 3H), 3.65 (s, 3H), 3.01+2.55 (dd+dd, 2H), 2.51-1.31 (m, 11H), 2.17 (m, 1H), 1.08 (d, 3H). HRMS calculated for C₃₄H₃₇N₃O₃Cl₂: 605.2212; found: 605.2212 (M+H).

### Example 631 (1r,2′S,4S)-4-(3-chloroanilino)-2′-{(2R)-3-[(3-chloro-1-methyl-1H-pyrrolo[3,2-b]pyridin-7-yl)oxy]-2-methylpropyl}-2′,3′-dihydrospiro[cyclohexane-1,1′-indene]-4-carboxylic acid

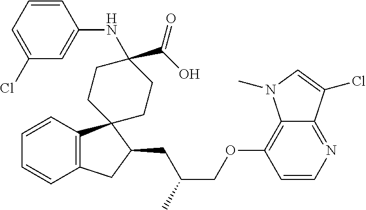 [SMILES: C[C@@H](COc1ccnc2c(Cl)cn(C)c12)C[C@H]1Cc2ccccc2[C@]12CC[C@@](Nc1cccc(Cl)c1)(C(=O)O)CC2]

Using General procedure 33a and Example 631A as the appropriate ester Example 631 was obtained. ¹H NMR (500 MHz, DMSO-d₆) δ ppm: 12.69 (br s, 1H), 8.19 (d, 1H), 7.61 (s, 1H), 7.41-7.09 (m, 4H), 7.02 (t, 1H), 6.79 (d, 1H), 6.62 (t, 1H), 6.53 (dm, 1H), 6.51 (dm, 1H), 6.18 (br s, 1H), 4.06/4.02 (dd+dd, 2H), 3.88 (s, 3H), 3.01/2.54 (dd+dd, 2H), 2.50-1.36 (m, 9H), 2.17 (m, 1H), 1.49/1.37 (m+m, 2H), 1.08 (d, 3H). HRMS calculated for C₃₃H₃₅N₃O₃Cl₂: 591.2056; found: 592.2121 (M+H).

### Example 632

### Example 632A 1-oxo-6,7,8,9-tetrahydro-5H-1λ⁵-cyclohepta[b]pyridine

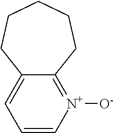 [SMILES: [O-][n+]1cccc2c1CCCCC2]

To a solution of 6,7,8,9-tetrahydro-5H-cyclohepta[b]pyridine (1.59 mL, 10.2 mmol, 1 eq) in DCM (45 mL), cooled to 0° C., was added mCPBA (2.58 g, 75% purity, 11.2 mmol, 1.1 eq) in portions and the mixture was warmed to rt and stirred for 3 h. The mixture was partitioned between DCM and 1 M aq. NaOH solution, and the organic phase was washed with brine, dried (MgSO₄) and concentrated in vacuo to afford Example 632A as a white solid that was used directly in the subsequent step without further purification. LRMS calculated for C₁₀H₁₃NO: 163; found: 164 (M+H). ¹H NMR (400 MHz, DMSO-d₆) δ ppm: 8.11-8.06 (m, 1H), 7.14-7.07 (m, 2H), 3.31-3.22 (m, 2H), 2.84-2.78 (m, 2H), 1.85-1.77 (m, 2H), 1.64-1.49 (m, 4H).

### Example 632B 4-chloro-6,7,8,9-tetrahydro-5H-cyclohepta[b]pyridine

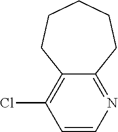 [SMILES: Clc1ccnc2c1CCCCC2]

A solution of Example 632A (1.70 g, 10.4 mmol, 1 eq) in POCl₃ (9.71 mL, 104 mmol, 10 eq) was heated at 90° C. for 3 h. The mixture was cooled to rt and added dropwise to a rapidly stirred solution of ice/water and then stirred for 30 min. The mixture was basified by the addition of aq. NH₃ solution, and then extracted with DCM. The combined organic extracts were washed with brine, dried (MgSO₄) and concentrated in vacuo. Purification by automated flash chromatography (CombiFlash Rf, 24 g RediSep™ silica cartridge) eluting with a gradient of 0-30% EtOAc (3% TEA) in heptane afforded Example 632B as a colourless oil (1.31 g, 7.21 mmol, 69%). LRMS calculated for C₁₀H₁₂ClN: 181; found: 182 (M+H). ¹H NMR (400 MHz, DMSO-d₆) δ ppm: 8.16 (d, J=5.4 Hz, 1H), 7.31 (d, J=5.4 Hz, 1H), 3.07-2.96 (m, 4H), 1.87-1.79 (m, 2H), 1.65-1.55 (m, 4H).

### Example 632 (1r,4R)-4-(3-chloroanilino)-2′-{(2R)-2-methyl-3-[(6,7,8,9-tetrahydro-5H-cyclohepta[b]pyridin-4-yl)oxy]propyl}spiro[cyclohexane-1,1′-indene]-4-carboxylic acid

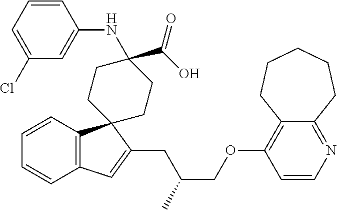 [SMILES: C[C@@H](COc1ccnc2c1CCCCC2)CC1=Cc2ccccc2[C@]12CC[C@@](Nc1cccc(Cl)c1)(C(=O)O)CC2]

Using General procedure 31c and Example 629E (147 mg, 0.35 mmol, 1 eq) as the appropriate alcohol and Example 632B (82 mg, 0.45 mmol, 1.3 eq) as the appropriate aryl chloride, Example 632 was obtained as a white powder (61.6 mg, 0.11 mmol, 31%). LRMS calculated for C₃₅H₃₉ClN₂O₃: 570; found: 571 (M+H). ¹H NMR (400 MHz, DMSO-d₆) δ ppm: 12.82 (br s, 1H), 8.04 (d, J=5.8 Hz, 1H), 7.65 (d, J=7.6 Hz, 1H), 7.30 (dd, J=7.4, 1.3 Hz, 1H), 7.23 (td, J=7.4, 0.9 Hz, 1H), 7.15-7.07 (m, 2H), 6.79 (d, J=5.8 Hz, 1H), 6.65 (t, J=2.1 Hz, 1H), 6.61-6.53 (m, 3H), 6.37 (br s, 1H), 4.00-3.90 (m, 2H), 2.91-2.71 (m, 3H), 2.61-2.32 (m, 5H), 2.26-2.12 (m, 4H), 2.12-2.00 (m, 1H), 1.79-1.61 (m, 2H), 1.61-1.23 (m, 4H), 1.10 (d, J=6.3 Hz, 3H), 0.95-0.85 (m, 1H), 0.82-0.73 (m, 1H).

### Example 633 (1r,2′S,4S)-4-(3-chloroanilino)-2′-{(2R)-2-methyl-3-[(6,7,8,9-tetrahydro-5H-cyclohepta[b]pyridin-4-yl)oxy]propyl}-2′,3′-dihydrospiro[cyclohexane-1,1′-indene]-4-carboxylic acid

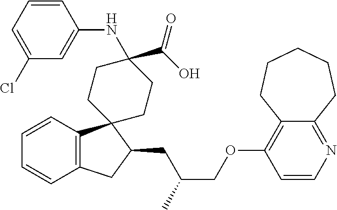 [SMILES: C[C@@H](COc1ccnc2c1CCCCC2)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and Example 632B (136 mg, 0.75 mmol, 2.2 eq) as the appropriate aryl chloride, Example 633 was obtained as a cream powder (52.7 mg, 0.09 mmol, 27%). LRMS calculated for C₃₅H₄₁ClN₂O₃: 572; found: 573 (M+H). ¹H NMR (400 MHz, DMSO-d₆) δ ppm: 8.05 (d, J=5.7 Hz, 1H), 7.46-7.37 (m, 1H), 7.20-7.00 (m, 3H), 6.96 (t, J=8.1 Hz, 1H), 6.79 (d, J=5.8 Hz, 1H), 6.77-6.69 (m, 1H), 6.66-6.57 (m, 1H), 6.47-6.40 (m, 1H), 5.91 (br s, 1H), 3.85 (d, J=6.0 Hz, 2H), 2.99 (dd, J=15.6, 7.0 Hz, 1H), 2.93-2.84 (m, 2H), 2.77-2.66 (m, 2H), 2.27-2.10 (m, 2H), 2.05-1.93 (m, 1H), 1.93-1.28 (m, 14H), 1.03 (d, J=6.6 Hz, 3H).

### Example 634

### Example 634A 4-chloro-1-oxo-6,7,8,9-tetrahydro-5H-1λ⁵-cyclohepta[b]pyridine

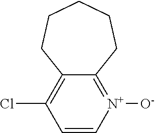 [SMILES: [O-][n+]1ccc(Cl)c2c1CCCCC2]

To a solution of Example 632B (726 mg, 4 mmol, 1 eq) in DCM (20 mL) was added mCPBA (1.01 g, 75% purity, 4.40 mmol, 1.1 eq) in portions and the mixture was stirred at rt for 2 h. 1 M aq. NaOH solution was added slowly and the mixture was stirred at rt for 1 h. The mixture was partitioned between DCM and water and the organic phase was washed with brine, dried (MgSO₄) and concentrated in vacuo to afford Example 634A as a white solid (770 mg, 3.9 mmol, 98%) that was used directly in the subsequent step without further purification. LRMS calculated for C₁₀H₁₂ClNO: 197; found: 198 (M+H). ¹H NMR (400 MHz, DMSO-d₆) δ ppm: 8.11 (d, J=7.0 Hz, 1H), 7.36 (d, J=7.0 Hz, 1H), 3.33-3.27 (m, 2H), 3.02-2.95 (m, 2H), 1.86-1.77 (m, 2H), 1.64-1.51 (m, 4H).

### Example 634B 4-chloro-6,7,8,9-tetrahydro-5H-cyclohepta[b]pyridin-9-ol

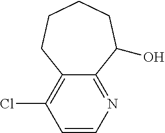 [SMILES: OC1CCCCc2c(Cl)ccnc21]

A solution of Example 634A (450 mg, 2.28 mmol, 1 eq) in AcOH (4 mL) was heated at 80° C. for 5 h and then allowed to cool to rt. MeOH (3 mL) and 2 M aq. NaOH solution (3 mL) were added and the mixture stirred vigorously at rt for 2 h. The mixture was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-39% EtOAc in heptane afforded racemate Example 634B as a solid (139 mg, 0.7 mmol, 31%). LRMS calculated for C₁₀H₁₂ClNO: 197; found: 198 (M+H). ¹H NMR (400 MHz, DMSO-d₆) δ ppm: 8.29 (d, J=5.4 Hz, 1H), 7.45 (d, J=5.4 Hz, 1H), 5.43 (d, J=4.2 Hz, 1H), 4.87 (ddd, J=9.7, 4.2, 2.0 Hz, 1H), 3.33-3.25 (m, 1H), 2.80-2.71 (m, 1H), 2.05-1.95 (m, 1H), 1.94-1.66 (m, 3H), 1.57-1.46 (m, 1H), 1.40-1.28 (m, 1H).

### Example 634C 4-chloro-9-[(4-methoxyphenyl)methoxy]-6,7,8,9-tetrahydro-5H-cyclohepta[b]pyridine

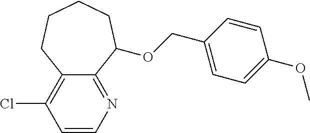 [SMILES: COc1ccc(COC2CCCCc3c(Cl)ccnc32)cc1]

To a solution of Example 634B (130 mg, 0.66 mmol, 1 eq), PMBCl (0.11 mL, 0.79 mmol, 1.2 eq) and TBAI (24 mg, 0.07 mmol, 0.1 eq) in DMF (3 mL) was added NaH (60% dispersion; 53 mg, 1.32 mmol, 2 eq) and the mixture was stirred at rt for 18 h under N₂. The mixture was partitioned between DCM and brine, dried (PTFE phase separator) and concentrated in vacuo. Purification by automated flash chromatography (CombiFlash Rf, 12 g RediSep™ silica cartridge) eluting with a gradient of 0-31% EtOAc in heptane afforded racemate Example 634C as a colourless oil (195 mg, 0.61 mmol, 93%). LRMS calculated for C₁₈H₂₀ClNO₂: 317; found: 318 (M+H). ¹H NMR (400 MHz, DMSO-d₆) δ ppm: 8.26 (d, J=5.3 Hz, 1H), 7.43 (d, J=5.3 Hz, 1H), 7.24-7.19 (m, 2H), 6.92-6.87 (m, 2H), 4.75 (dd, J=6.7, 1.4 Hz, 1H), 4.33 (d, J=11.4 Hz, 1H), 4.26 (d, J=11.4 Hz, 1H), 3.74 (s, 3H), 3.18-3.06 (m, 2H), 2.16-2.03 (m, 2H), 1.94-1.83 (m, 1H), 1.77-1.54 (m, 2H), 1.37-1.24 (m, 1H).

### Example 634D (1r,2′S,4S)-4-(3-chloroanilino)-2′-[(2R)-3-({9-[(4-methoxyphenyl)methoxy]-6,7,8,9-tetrahydro-5H-cyclohepta[b]pyridin-4-yl}oxy)-2-methylpropyl]-2′,3′-dihydrospiro[cyclohexane-1,1′-indene]-4-carboxylic acid

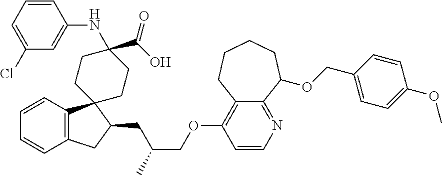 [SMILES: COc1ccc(COC2CCCCc3c(OC[C@H](C)C[C@H]4Cc5ccccc5[C@]45CC[C@@](Nc4cccc(Cl)c4)(C(=O)O)CC5)ccnc32)cc1]

Using General procedure 31c and Preparation 10b1 (110 mg, 0.25 mmol, 1 eq) as the appropriate alcohol and Example 634C (174 mg, 0.55 mmol, 2.2 eq) as the appropriate aryl chloride, a mixture of diastereoisomers, Example 634D was obtained as a colourless glass (135 mg, 0.19 mmol, 77%). LRMS calculated for C₄₃H₄₉ClN₂O₅: 708; found: 709 (M+H). ¹H NMR (400 MHz, DMSO-d₆) δ ppm: 8.53-8.43 (m, 1H), 7.52-7.29 (m, 4H), 7.23-7.12 (m, 3H), 7.08-7.02 (m, 1H), 6.95-6.89 (m, 2H), 6.65-6.61 (m, 1H), 6.57-6.50 (m, 2H), 5.00-4.89 (m, 1H), 4.65-4.51 (m, 2H), 4.17 (d, J=5.7 Hz, 1H), 3.75 (s, 3H), 3.16-2.96 (m, 2H), 2.66-2.38 (m, 2H), 2.21-1.17 (m, 19H), 1.06 (d, J=6.5 Hz, 3H).

### Example 634 (1r,2′S,4S)-4-(3-chloroanilino)-2′-{(2R)-3-[(9-hydroxy-6,7,8,9-tetrahydro-5H-cyclohepta[b]pyridin-4-yl)oxy]-2-methylpropyl}-2′,3′-dihydrospiro[cyclohexane-1,1′-indene]-4-carboxylic acid

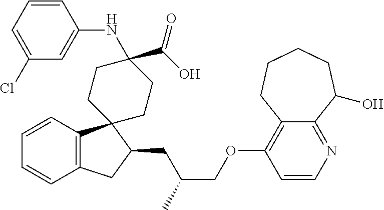 [SMILES: C[C@@H](COc1ccnc2c1CCCCC2O)C[C@H]1Cc2ccccc2[C@]12CC[C@@](Nc1cccc(Cl)c1)(C(=O)O)CC2]

Using General procedure 28b and Example 634D (135 mg, 0.19 mmol, 1 eq) as the appropriate PMB derivative, and then purification by reverse phase automated flash chromatography at pH 4 (CombiFlash Rf, C18 13 g RediSep column) eluting with a gradient of 10-75% MeCN in water, a mixture of diastereoisomers, Example 634 was obtained as a white powder (33.4 mg, 0.06 mmol, 30%). LRMS calculated for C₃₅H₄₁ClN₂O₄: 588; found: 589 (M+H). ¹H NMR (400 MHz, DMSO-d₆) δ ppm: 8.24-8.19 (m, 1H), 7.42-7.34 (m, 1H), 7.24-7.11 (m, 3H), 7.05 (t, J=8.1 Hz, 1H), 6.97 (d, J=5.8 Hz, 1H), 6.63 (t, J=2.1 Hz, 1H), 6.57-6.51 (m, 2H), 5.57 (br s, 1H), 4.72-4.65 (m, 1H), 3.97-3.89 (m, 2H), 3.26-3.16 (m, 1H), 3.00 (dd, J=15.6, 7.1 Hz, 1H), 2.58-2.39 (m, 2H), 2.33-2.21 (m, 1H), 2.19-1.80 (m, 8H), 1.80-1.61 (m, 3H), 1.53-1.23 (m, 5H), 1.10-0.94 (m, 4H).

### Example 635

### Example 635A 8-oxo-3,4-dihydro-2H-8λ⁵-pyrano[2,3-b]pyridine

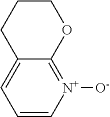 [SMILES: [O-][n+]1cccc2c1OCCC2]

To a solution of 3,4-dihydro-2H-pyrano[2,3-b]pyridine (225 mg, 1.66 mmol, 1 eq) in DCM (5 mL), cooled to 0° C., was added mCPBA (410 mg, 77% purity, 1.83 mmol, 1.1 eq) in portions and the mixture was stirred at 0° C. for 3 h, then at rt for 18 h. The mixture was cooled to 0° C., sat. aq. NaHCO₃ solution was added and the mixture was stirred for 30 min. The mixture was partitioned between DCM and 2 M aq. NaOH solution. The aq. phase was extracted further with DCM/iPrOH (3:1), and the combined organic extracts were washed with brine, dried (MgSO₄) and concentrated in vacuo to afford Example 635A as a cream gum that was used directly in the subsequent step without further purification. LRMS calculated for C₈H₉NO₂: 151; found: 152 (M+H).

### Example 635B 5-chloro-3,4-dihydro-2H-pyrano[2,3-b]pyridine

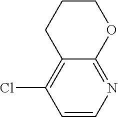 [SMILES: Clc1ccnc2c1CCCO2]

A solution of Example 635A (251 mg, 1.66 mmol, 1 eq) in POCl₃ (1.55 mL, 16.6 mmol, 10 eq) was heated at 90° C. for 18 h. The mixture was cooled to rt and added dropwise to a rapidly stirred solution of ice/water and stirred for 30 min. The mixture was basified with aq. NH₃ solution and extracted with DCM. The organic phase was washed with brine, dried (MgSO₄) and concentrated in vacuo. Purification by automated flash chromatography (CombiFlash Rf, 12 g RediSep™ silica cartridge) eluting with a gradient of 0-50% EtOAc in heptane afforded Example 635B as a colourless solid (59 mg, 0.35 mmol, 21%). LRMS calculated for C₈H₈ClNO: 169; found: 170 (M+H). ¹H NMR (400 MHz, DMSO-d₆) δ ppm: 7.98 (dt, J=5.4, 0.9 Hz, 1H), 7.11 (d, J=5.3 Hz, 1H), 4.30-4.25 (m, 2H), 2.79-2.73 (m, 2H), 2.00-1.93 (m, 2H).

### Example 635 (1r,2′S,4S)-4-(3-chloroanilino)-2′-{(2R)-3-[(3,4-dihydro-2H-pyrano[2,3-b]pyridin-5-yl)oxy]-2-methylpropyl}-2′,3′-dihydrospiro[cyclohexane-1,1′-indene]-4-carboxylic acid

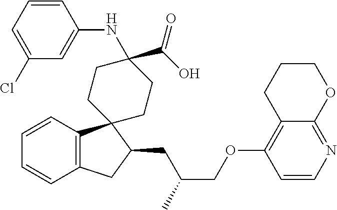 [SMILES: C[C@@H](COc1ccnc2c1CCCO2)C[C@H]1Cc2ccccc2[C@]12CC[C@@](Nc1cccc(Cl)c1)(C(=O)O)CC2]

Using General procedure 31c and Preparation 10b1 (100 mg, 0.23 mmol, 1 eq) as the appropriate alcohol and Example 635B (57.6 mg, 0.34 mmol, 1.5 eq) as the appropriate aryl chloride, Example 635 was obtained as a white powder (7.5 mg, 0.01 mmol, 6%). LRMS calculated for C₃₃H₃₇ClN₂O₄: 560; found: 561 (M+H). ¹H NMR (400 MHz, DMSO-d₆) δ ppm: 7.87 (d, J=5.7 Hz, 1H), 7.42-7.36 (m, 1H), 7.23-7.12 (m, 3H), 7.06 (t, J=8.1 Hz, 1H), 6.65-6.60 (m, 2H), 6.58-6.52 (m, 2H), 4.20-4.14 (m, 2H), 3.94-3.86 (m, 2H), 3.00 (dd, J=15.7, 7.1 Hz, 1H), 2.59-2.40 (m, 4H), 2.18-1.67 (m, 9H), 1.54-1.29 (m, 4H), 1.04 (d, J=6.6 Hz, 3H).

### Example 636 (1r,4R)-4-(3-chloroanilino)-2′-{(2R)-2-methyl-3-[(5,6,7,8-tetrahydroquinazolin-4-yl)oxy]propyl}spiro[cyclohexane-1,1′-indene]-4-carboxylic acid

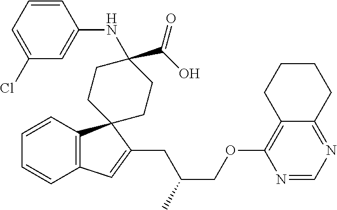 [SMILES: C[C@@H](COc1ncnc2c1CCCC2)CC1=Cc2ccccc2[C@]12CC[C@@](Nc1cccc(Cl)c1)(C(=O)O)CC2]

Using General procedure 31c and Example 629E (140 mg, 0.33 mmol, 1 eq) as the appropriate alcohol and 4-chloro-5,6,7,8-tetrahydroquinazoline (83 mg, 0.49 mmol, 1.5 eq) as the appropriate aryl chloride, Example 636 was obtained as a white powder (100 mg, 0.18 mmol, 55%). LRMS calculated for C₃₃H₃₆ClN₃O₃: 557; found: 558 (M+H). ¹H NMR (400 MHz, DMSO-d₆) δ ppm: 12.81 (br s, 1H), 8.44 (s, 1H), 7.66 (d, J=7.5 Hz, 1H), 7.30 (dd, J=7.4, 1.3 Hz, 1H), 7.23 (td, J=7.4, 0.9 Hz, 1H), 7.15-7.06 (m, 2H), 6.64 (t, J=2.1 Hz, 1H), 6.60-6.54 (m, 3H), 6.37 (br s, 1H), 4.33-4.21 (m, 2H), 2.69-2.63 (m, 2H), 2.49-2.33 (m, 6H), 2.25-2.04 (m, 5H), 1.78-1.59 (m, 4H), 1.07 (d, J=6.1 Hz, 3H), 0.94-0.77 (m, 2H).

### Example 637 (1r,2′S,4S)-4-(3-chloroanilino)-2′-{(2R)-2-methyl-3-[(5,6,7,8-tetrahydroquinazolin-4-yl)oxy]propyl}-2′,3′-dihydrospiro[cyclohexane-1,1′-indene]-4-carboxylic acid

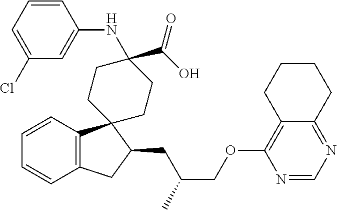 [SMILES: C[C@@H](COc1ncnc2c1CCCC2)C[C@H]1Cc2ccccc2[C@]12CC[C@@](Nc1cccc(Cl)c1)(C(=O)O)CC2]

Using General procedure 31c and Preparation 10b1 (120 mg, 0.27 mmol, 1 eq) as the appropriate alcohol and 4-chloro-5,6,7,8-tetrahydroquinazoline (101 mg, 0.6 mmol, 2.2 eq) as the appropriate aryl chloride, Example 637 was obtained as a yellow powder (28.7 mg, 0.05 mmol, 19%). LRMS calculated for C₃₃H₃₈ClN₃O₃: 559; found: 560 (M+H). ¹H NMR (400 MHz, DMSO-d₆) δ ppm: 8.92 (s, 1H), 7.42-7.35 (m, 1H), 7.23-7.12 (m, 3H), 7.06 (t, J=8.1 Hz, 1H), 6.63 (t, J=2.1 Hz, 1H), 6.59-6.52 (m, 2H), 4.40-4.30 (m, 2H), 2.99 (dd, J=15.7, 7.1 Hz, 1H), 2.86-2.79 (m, 2H), 2.58-2.38 (m, 4H), 2.17-1.64 (m, 11H), 1.53-1.32 (m, 4H), 1.04 (d, J=6.4, 3H).

### Example 638

### Example 638A methyl (2R)-2-methyl-6-oxocyclohexane-1-carboxylate

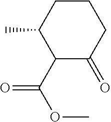

A suspension of Cu(OTf)₂ (41 mg, 0.11 mmol, 0.01 eq) and N,N-bis-[(R)-1-phenylethyl]dibenzo[d,f][1,3,2]dioxaphosphepin-6-amine (101 mg, 0.23 mmol, 0.01 eq) in toluene (35 mL) was stirred at rt for 30 min under N₂. The solution was cooled to 0° C. for 10 min, followed by the addition of cyclohexenone (2.22 mL, 22.89 mmol, 1 eq). Dimethyl zinc (12.59 mL, 2 M in toluene, 25.17 mmol, 1.1 eq) was added dropwise over 20 min and the mixture was stirred at 0° C. for 30 min. The solution was cooled to −78° C. and stirred for 20 min. MeLi (15.73 mL, 1.6 M in Et₂O, 25.17 mmol, 1.1 eq) was added dropwise over 10 min and stirring continued at −78° C. for 10 min. A solution of methyl 1H-imidazole-1-carboxylate (6.65 mL, 4.3 M, 28.61 mmol, 1.25 eq) in toluene was added dropwise over 10 min, the mixture was then stirred at −78° C. for a further 10 min and then allowed to slowly warm to rt and stirred for 4 h. The reaction was quenched with sat. aq. NH₄Cl solution and extracted with EtOAc. The combined organic extracts were dried (MgSO₄) and concentrated in vacuo. Purification by automated flash chromatography (Combiflash Rf, Silica 80 g, RediSep™ cartridge) eluting with a gradient of 0-13% EtOAc in heptane afforded Example 638A as a colourless oil (2.56 g, 15.03 mmol, 66%).

### Example 638B (5R)-5-methyl-5,6,7,8-tetrahydroquinazolin-4 (3H)-one

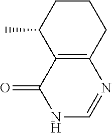

To a solution of Example 638A (2.50 g, 14.69 mmol, 1 eq) in toluene (40 mL) was added 1,1,1-trimethyl-N-(triphenylphosphoranylidene)silanamine (3.59 g, 10.28 mmol, 0.7 eq) and PTSA (28 mg, 0.15 mmol, 0.01 eq) and the mixture was refluxed for 5 h. The mixture was allowed to cool to rt, poured into sat. aq. NaHCO₃ solution and extracted with EtOAc. The organic phase was dried (MgSO₄), concentrated in vacuo and purified by automated flash chromatography (Combiflash Rf, Silica 40 g, RediSep™ cartridge) eluting with a gradient of 0-15% EtOAc in heptane. Then it was dissolved in formamide (1.89 mL, 47.28 mmol, 5 eq), NH₄HCO₂ (2.98 g, 47.3 mmol, 5 eq) was added and the mixture was stirred at 150° C. for 4 h. The mixture was cooled to rt and partitioned between DCM/iPrOH(5:1) and water, and the organic phase was washed with brine, dried (MgSO₄) and concentrated in vacuo. Purification by automated flash chromatography (CombiFlash Rf, 24 g RediSep™ silica cartridge) eluting with a gradient of 0-10% MeOH in DCM afforded Example 638B as a colourless gum (70 mg, 0.43 mmol, 5%). LRMS calculated for C₉H₁₂N₂O: 164; found: 165 (M+H). ¹H NMR (400 MHz, DMSO-d₆) δ ppm: 12.18 (s, 1H), 7.94 (s, 1H), 2.87-2.77 (m, 1H), 2.50-2.45 (m, 2H), 1.82-1.51 (m, 4H), 1.10 (d, J=6.9 Hz, 3H).

### Example 638C (5R)-4-chloro-5-methyl-5,6,7,8-tetrahydroquinazoline

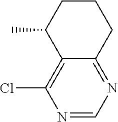

A solution of Example 638B (70 mg, 0.43 mmol, 1 eq) in POCl₃ (0.4 mL, 4.26 mmol, 10 eq) was heated at 90° C. for 2 h. The mixture was cooled to rt and added dropwise to a rapidly stirred solution of ice/water and then basified with aq. NH₃ solution. The mixture was extracted with DCM and the combined organic extracts were washed with brine, dried (MgSO₄) and concentrated in vacuo to afford Example 638C as a yellow oil (68 mg, 0.37 mmol, 87%). LRMS calculated for C₉H₁₁ClN₂: 182; found: 183 (M+H). ¹H NMR (400 MHz, DMSO-d₆) δ ppm: 8.74 (s, 1H), 3.18-3.10 (m, 1H), 2.95-2.85 (m, 1H), 2.84-2.73 (m, 1H), 1.97-1.72 (m, 4H), 1.23 (d, J=7.0 Hz, 3H).

### Example 638 (1r,2′S,4S)-4-(3-chloroanilino)-2′-[(2R)-2-methyl-3-{[(5R)-5-methyl-5,6,7,8-tetrahydroquinazolin-4-yl]oxy}propyl]-2′,3′-dihydrospiro[cyclohexane-1,1′-indene]-4-carboxylic acid

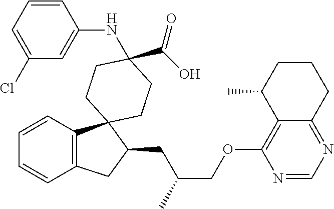 [SMILES: C[C@@H](COc1ncnc2c1[C@H](C)CCC2)C[C@H]1Cc2ccccc2[C@]12CC[C@@](Nc1cccc(Cl)c1)(C(=O)O)CC2]

Using General procedure 31c and Preparation 10b1 (125 mg, 0.29 mmol, 1 eq) as the appropriate alcohol and Example 638C (69 mg, 0.38 mmol, 1.3 eq) as the appropriate aryl chloride, purification by prep RP-HPLC at pH 4 afforded Example 638 as an off-white glass (11 mg, 0.02 mmol, 6%). LRMS calculated for C₃₄H₄₀ClN₃O₃: 573; found: 574 (M+H). ¹H NMR (400 MHz, DMSO-d₆) δ ppm: 8.49 (s, 1H), 7.43-7.34 (m, 1H), 7.25-7.11 (m, 3H), 7.05 (t, J=8.0 Hz, 1H), 6.64-6.59 (m, 1H), 6.58-6.51 (m, 2H), 4.26 (dd, J=10.4, 6.4 Hz, 1H), 4.16 (dd, J=10.5, 5.7 Hz, 1H), 3.05-2.89 (m, 2H), 2.77-2.59 (m, 2H), 2.48-2.39 (m, 1H), 2.18-1.58 (m, 11H), 1.53-1.29 (m, 4H), 1.08 (d, J=6.9 Hz, 3H), 1.03 (d, J=6.7 Hz, 3H).

### Example 639 and Example 640

### Example 639A 3,5-dimethylquinolin-2 (1H)-one

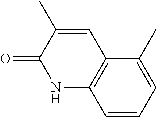

To a solution of 2-bromo-3-methylaniline (5.00 g, 26.87 mmol, 1 eq) and methyl methacrylate (3.74 mL, 34.94 mmol, 1.3 eq) in DMF (50 mL) was added tri-o-tolylphosphine (818 mg, 2.69 mmol, 0.1 eq) and DIPEA (7.02 mL, 40.31 mmol, 1.5 eq) and the mixture was sparged with N₂. Pd(OAc)₂ (302 mg, 1.34 mmol, 0.05 eq) was added and the mixture was heated at 100° C. under N₂ for 72 h. The mixture was allowed to cool to rt and partitioned between DCM and water. The layers were separated, and the organic phase was washed with water, dried (MgSO₄) and concentrated in vacuo to form a precipitate that was filtered and washed with Et₂O. The filtrate was concentrated and purified by automated flash chromatography (Combiflash Rf, Silica 40 g RediSep™ cartridge) eluting with a gradient of 0-61% EtOAc in heptane and triturated with Et₂O to afford Example 639A as a cream powder (1.46 g, 8.4 mmol, 31%). LRMS calculated for C₁₁H₁₁NO: 173; found: 174 (M+H). ¹H NMR (400 MHz, DMSO-d₆) δ ppm: 11.71 (s, 1H), 7.93-7.90 (m, 1H), 7.33-7.27 (m, 1H), 7.16-7.12 (m, 1H), 7.01-6.96 (m, 1H), 2.50 (s, 3H), 2.13 (d, J=1.2 Hz, 3H).

### Example 639B 2-chloro-3,5-dimethylquinoline

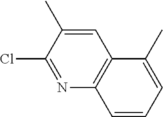

POCl₃ (6 mL) was added dropwise to Example 639A (1.46 g, 8.43 mmol, 1 eq) cooled to 0° C. and then the mixture was stirred at 90° C. for 2.5 h under N₂. The mixture was allowed to cool to rt, and the resulting suspension was added dropwise to vigorously stirred ice-water. The precipitate was collected by filtration, washed with water and dried in vacuo to afford Example 639B as a yellow powder (1.39 g, 7.25 mmol, 86%). LRMS calculated for C_{II}H₁₀ClN: 191; found: 192 (M+H). ¹H NMR (400 MHz, DMSO-d₆) δ ppm: 8.48-8.44 (m, 1H), 7.80-7.74 (m, 1H), 7.67-7.61 (m, 1H), 7.49-7.44 (m, 1H), 2.69-2.65 (m, 3H), 2.54 (d, J=1.0 Hz, 3H).

### Example 639C 3,5-dimethylquinoline

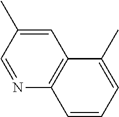

To a solution of Example 639B (1.39 g, 7.25 mmol, 1 eq) in EtOH (60 mL) was added 10% Pd/C under a N₂ atmosphere. The mixture was evacuated and backfilled N₂ (×3), then evacuated and backfilled with H₂ and shaken at rt under an atmosphere of H₂ until no further conversion was observed. The mixture was filtered through celite, washed with EtOH and the filtrate was concentrated in vacuo and triturated with Et₂O. The solid was suspended in DCM, washed with 1 M aq. NaOH solution and the organic phase dried (MgSO₄) and concentrated to afford Example 639C as a brown oil. LRMS calculated for C₁₁H₁₁N: 157; found: 158 (M+H). ¹H NMR (400 MHz, DMSO-d₆) δ ppm: 8.78 (d, J=2.1 Hz, 1H), 8.26-8.22 (m, 1H), 7.85-7.80 (m, 1H), 7.60-7.53 (m, 1H), 7.44-7.39 (m, 1H), 2.67-2.64 (m, 3H), 2.54-2.52 (m, 3H).

### Example 639D 3,5-dimethyl-1-oxo-1λ⁵-quinoline

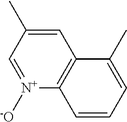

To a solution of Example 639C (1.14 g, 7.25 mmol, 1 eq) in DCM (20 mL) cooled to 0° C., was added mCPBA (77% purity, 2.44 g, 10.88 mmol, 1.5 eq) portionwise and the mixture was allowed to warm to rt and stirred for 2 h. 2 M aq. NaOH solution was added, and the mixture was stirred for 10 min. The layers were separated, and the aq. phase extracted with DCM. The combined organic extracts were dried (MgSO₄) and concentrated in vacuo. Trituration with Et₂O afforded Example 639D as a cream powder (893 mg, 5.16 mmol, 71%). LRMS calculated for C_{II}H₁₁NO: 173; found: 174 (M+H). ¹H NMR (400 MHz, DMSO-d₆) δ ppm: 8.57-8.53 (m, 1H), 8.38-8.32 (m, 1H), 7.85-7.80 (m, 1H), 7.64-7.58 (m, 1H), 7.56-7.51 (m, 1H), 2.68-2.64 (m, 3H), 2.46-2.43 (m, 3H).

### Example 639E 4-chloro-3,5-dimethylquinoline

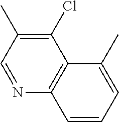

Example 639D (1.02 g, 5.87 mmol, 1 eq) was added portionwise to POCl₃ (6 mL, 64.37 mmol, 10.97 eq) cooled to 0° C. The mixture was then heated at 90° C. and stirred under N₂ for 2 h. A mixture of regioisomers was observed. The mixture was allowed to cool to rt and then quenched by adding dropwise to a vigorously stirred ice-water. The aq. phase was basified with 2 M aq. NaOH solution and then extracted with DCM. The organic phase was dried (MgSO₄) and concentrated in vacuo. The regioisomers were separated by automated flash chromatography (Combiflash Rf, Silica 40 g RediSep™ cartridge) eluting with a gradient of 0-10% MeOH in DCM. The regioisomer eluting later was collected and further purified by reverse phase automated flash chromatography (Combiflash Rf, C18 13 g RediSep column) eluting with a gradient of 10-100% MeCN in water to obtain Example 639E as a yellow powder (357 mg, 1.86 mmol, 32%). LRMS calculated for C_{II}H₁₀ClN: 191; found: 192 (M+H). ¹H NMR (400 MHz, DMSO-d₆) δ ppm: 8.80 (s, 1H), 7.94-7.88 (m, 1H), 7.66-7.59 (m, 1H), 7.52-7.46 (m, 1H), 3.04-2.99 (m, 3H), 2.52-2.50 (m, 3H).

### Example 639F 4-(benzyloxy)-3,5-dimethylquinoline

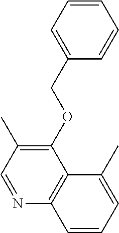 [SMILES: Cc1cnc2cccc(C)c2c1OCc1ccccc1]

To a solution of BnOH (0.58 mL, 5.6 mmol, 2.5 eq) in THE (15 mL) cooled to 0° C. was added NaH (60% dispersion; 161 mg, 6.72 mmol, 3 eq) and the mixture was stirred for 10 min. A solution of Example 639E (429 mg, 2.24 mmol, 1 eq) in THE (10 mL) was added and the mixture was refluxed for 18 h. The mixture was allowed to cool to rt and then cooled to 0° C., NaH (60% dispersion; 161 mg, 6.72 mmol, 3 eq) was added again and the mixture was stirred for 10 min and then refluxed until no further conversion was observed. The mixture was partitioned between EtOAc and water. The layers were separated, and the organic phase was dried (MgSO₄) and concentrated in vacuo. Purification by automated flash chromatography (Combiflash Rf, Silica 24 g RediSep™ cartridge) eluting with a gradient of 0-50% EtOAc in hexane afforded Example 639F as a yellow oil (173.5 mg, 0.66 mmol, 29%). LRMS calculated for C₁₈H₁₇NO: 263; found 264 (M+H). ¹H NMR (400 MHz, DMSO-d₆) δ ppm: 8.76 (s, 1H), 7.87-7.82 (m, 1H), 7.59-7.53 (m, 3H), 7.50-7.43 (m, 2H), 7.43-7.37 (m, 1H), 7.37-7.32 (m, 1H), 5.00 (s, 2H), 2.85-2.81 (m, 3H), 2.47-2.44 (s, 3H).

### Example 639G 3,5-dimethyl-5,6,7,8-tetrahydro-1H-quinolin-4-one

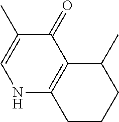

To a solution of Example 639F (302 mg, 1.74 mmol, 1 eq) in TFA (5 mL) was added catalytic PtO₂. The mixture was evacuated and backfilled N₂ (×3), then evacuated and backfilled with H₂ and shaken at 50° C. under an atmosphere of H₂ until no further conversion was observed. The mixture was diluted with DCM and washed with water. The organic phase was filtered through a celite cartridge and the filtrate was washed with dilute aq. NaOH solution. The aq. phase was basified, extracted with DCM/IPA (3:1) and the combined organic extracts were dried (MgSO₄) and concentrated in vacuo. Purification by automated flash chromatography (Combiflash Rf, Silica 12 g RediSep™ cartridge) eluting with a gradient of 0-20% MeOH in DCM afforded a racemate, Example 639G, isolated as a colourless gum (181 mg, 1.02 mmol, 59%). LRMS calculated for C₁₁H₁₅NO: 177; found: 178 (M+H). ¹H NMR (400 MHz, DMSO-d₆) δ ppm: 10.97 (br s, 1H), 7.45-7.40 (m, 1H), 2.93-2.83 (m, 1H), 2.51-2.45 (m, 2H), 1.85-1.80 (m, 3H), 1.79-1.64 (m, 2H), 1.64-1.52 (m, 2H), 1.06 (d, J=6.8 Hz, 3H).

### Example 639H methyl (1r,2′S,4S)-4-(3-chloroanilino)-2′-[(2R)-3-(3,5-dimethyl-4-oxo-5,6,7,8-tetrahydroquinolin-1 (4H)-yl)-2-methylpropyl]-2′,3′-dihydrospiro[cyclohexane-1,1′-indene]-4-carboxylate, diastereoisomer 1

and

### Example 640A methyl (1r,2′S,4S)-4-(3-chloroanilino)-2′-[(2R)-3-(3,5-dimethyl-4-oxo-5,6,7,8-tetrahydroquinolin-1 (4H)-yl)-2-methylpropyl]-2′,3′-dihydrospiro[cyclohexane-1,1′-indene]-4-carboxylate, diastereoisomer 2

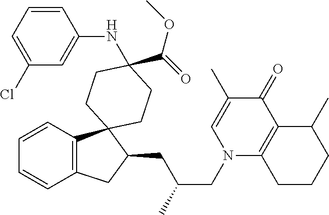 [SMILES: COC(=O)[C@]1(Nc2cccc(Cl)c2)CC[C@@]2(CC1)c1ccccc1C[C@@H]2C[C@@H](C)Cn1cc(C)c(=O)c2c1CCCC2C]

Using General procedure 30a and Preparation 10b1 as the appropriate indane and Example 639G as the appropriate alcohol, under refluxing conditions, a mixture of diastereoisomers was obtained. The diastereoisomers were separated by chiral chromatography. Column: AD, 50×500 mm, 20 μm, Eluents: EtOH/heptane. The diastereoisomer eluting earlier was collected as Example 639H. ¹H NMR (500 MHz, DMSO-d₆) δ ppm: 7.53 (s, 1H), 7.37 (d, 1H), 7.20-7.10 (m, 3H), 7.07 (t, 1H), 6.62 (t, 1H), 6.57 (dm, 1H), 6.44 (dm, 1H), 6.31 (s, 1H), 3.80+3.66 (m+m, 2H), 3.65 (s, 3H), 2.98-1.12 (m, 21H), 1.78 (s, 3H), 0.97 (d, 3H), 0.85 (d, 3H).

The diastereoisomer eluting later was collected as Example 640A. ¹H NMR (400 MHz, DMSO-d₆) δ ppm: 7.55 (s, 1H), 7.38 (d, 1H), 7.18-7.13 (m, 3H), 7.08 (t, 1H), 6.62 (t, 1H), 6.59 (dd, 1H), 6.45 (dd, 1H), 6.31 (s, 1H), 3.83/3.62 (dd+dd, 2H), 3.66 (s, 3H), 2.94/2.38 (dd+dd, 2H), 2.92 (m, 1H), 2.72-1.17 (m, 16H), 2.07 (m, 1H), 1.91 (m, 1H), 1.80 (s, 3H), 1.03 (d, 3H), 0.86 (d, 3H). HRMS calculated for C₃₇H₄₅N₂O₃Cl: 600.3119; found: 601.3193 (M+H).

### Example 639 (1r,2′S,4S)-4-(3-chloroanilino)-2′-[(2R)-3-(3,5-dimethyl-4-oxo-5,6,7,8-tetrahydroquinolin-1 (4H)-yl)-2-methylpropyl]-2′,3′-dihydrospiro[cyclohexane-1,1′-indene]-4-carboxylic acid, diastereoisomer 1

and

### Example 640 (1r,2′S,4S)-4-(3-chloroanilino)-2′-[(2R)-3-(3,5-dimethyl-4-oxo-5,6,7,8-tetrahydroquinolin-1 (4H)-yl)-2-methylpropyl]-2′,3′-dihydrospiro[cyclohexane-1,1′-indene]-4-carboxylic acid, diastereoisomer 2

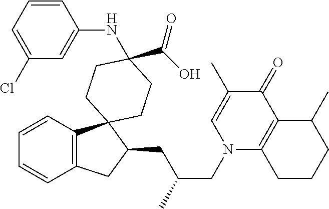 [SMILES: Cc1cn(C[C@H](C)C[C@H]2Cc3ccccc3[C@]23CC[C@@](Nc2cccc(Cl)c2)(C(=O)O)CC3)c2c(c1=O)C(C)CCC2]

Using General procedure 33a and Example 639H as the appropriate ester Example 639 was obtained. ¹H NMR (500 MHz, DMSO-d₆) δ ppm: 12.72 (br s, 1H), 7.53 (q, 1H), 7.41-7.09 (m, 4H), 7.06 (t, 1H), 6.64 (t, 1H), 6.55 (dm, 1H), 6.53 (dm, 1H), 6.22 (br s, 1H), 3.77/3.65 (dd+dd, 2H), 2.93/2.39 (dd+dd, 2H), 2.92 (m, 1H), 2.63/2.50 (m+m, 2H), 2.50-1.20 (m, 12H), 2.06 (m, 1H), 1.91 (m, 1H), 1.78 (d, 3H), 1.23/1.17 (m+m, 2H), 0.98 (d, 3H), 0.85 (d, 3H). HRMS calculated for C₃₆H₄₃N₂O₃Cl: 586.2962; found: 587.3032 (M+H).

Using General procedure 33a and Example 640A as the appropriate ester Example 640 was obtained. ¹H NMR (500 MHz, DMSO-d₆) δ ppm: 12.65 (br s, 1H), 7.54 (q, 1H), 7.4-7.09 (m, 4H), 7.06 (t, 1H), 6.64 (t, 1H), 6.55 (dm, 1H), 6.53 (dm, 1H), 6.21 (br s, 1H), 3.84/3.61 (dd+dd, 2H), 2.93/2.39 (dd+dd, 2H), 2.92 (m, 1H), 2.70/2.46 (m+m, 2H), 2.48-1.24 (m, 12H), 2.06 (m, 1H), 1.90 (m, 1H), 1.79 (d, 3H), 1.23/1.18 (m+m, 2H), 1.03 (d, 3H), 0.84 (d, 3H). HRMS calculated for C₃₆H₄₃N₂O₃Cl: 586.2962; found: 587.3033 (M+H).

### Example 641 and Example 642

### Example 641A ethyl 4-(benzyloxy)-2-chloropyridine-3-carboxylate

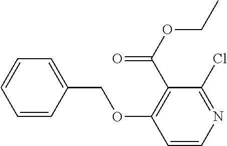 [SMILES: CCOC(=O)c1c(OCc2ccccc2)ccnc1Cl]

NaH (60% dispersion; 2.10 g, 52.5 mmol, 1.1 eq) was added portionwise to a solution of ethyl 2,4-dichloropyridine-3-carboxylate (10.5 g, 47.72 mmol, 1 eq) and BnOH (5.21 mL, 50.10 mmol, 1.05 eq) in DMF (100 mL), cooled to 0° C., under N₂. The mixture was stirred at 0° C. for 1.5 h and then quenched by the dropwise addition of sat. aq. NH₄Cl solution, diluted with water and extracted with EtOAc. The combined organic extracts were washed with brine, dried (MgSO₄) and concentrated in vacuo. Purification by automated flash chromatography (CombiFlash Rf, 24 g RediSep™ silica cartridge) eluting with a gradient of 0-40% EtOAc in heptane afforded Example 641A as a white solid (11.5 g, 39.42 mmol, 83%). LRMS calculated for: C₁₅H₁₄NO₃Cl: 291; found: 292 (M+H). ¹H NMR (400 MHz, CDCl₃) δ ppm: 8.25 (d, J=5.8 Hz, 1H), 7.42-7.30 (m, 5H), 6.84 (d, J=5.8 Hz, 1H), 5.19 (s, 2H), 4.42 (q, J=7.1 Hz, 2H), 1.35 (t, J=7.1 Hz, 3H).

### Example 641B [4-(benzyloxy)-2-chloropyridin-3-yl]methanol

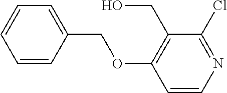 [SMILES: OCc1c(OCc2ccccc2)ccnc1Cl]

LAH (46.8 mL, 1 M in THF, 46.8 mmol, 1.2 eq) was added dropwise to a solution of Example 641A (11.5 g, 39.42 mmol, 1 eq) in THE (100 mL) under N₂ at 0° C., and then stirring was continued for 1 h at 0° C. and then at rt. On completion, the reaction was carefully quenched by the dropwise addition of water (1.8 mL), 15% aq. NaOH solution (1.8 mL) and water (5.4 mL) and then diluted with EtOAc. MgSO₄ was added and then stirring was continued for 1 h and then the mixture was filtered through celite and the filter cake was washed with EtOAc. The filtrate was dried (MgSO₄) and concentrated in vacuo. Purification by automated flash chromatography (CombiFlash Rf, 120 g RediSep™ silica cartridge) eluting with a gradient of 0-70% EtOAc in heptane afforded Example 641B as a white solid (6.4 g, 25.63 mmol, 65%). LRMS calculated for C₁₃H₁₂NO₂Cl: 249; found: 250 (M+H). ¹H NMR (400 MHz, CDCl₃) δ ppm: 8.21 (d, J=5.7 Hz, 1H), 7.45-7.35 (m, 5H), 6.85 (d, J=5.7 Hz, 1H), 5.18 (s, 2H), 4.89 (d, J=7.0 Hz, 2H), 2.27 (t, J=7.0 Hz, 1H).

### Example 641C 4-(benzyloxy)-2-chloropyridine-3-carbaldehyde

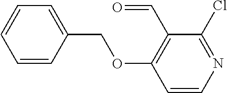 [SMILES: O=Cc1c(OCc2ccccc2)ccnc1Cl]

To a solution of Example 641B (6.4 g, 25.63 mmol, 1 eq) in DCM (100 mL) at 0° C. was added DMP (11.03 g, 26.00 mmol, 1.0 eq) in portions, and then the mixture was stirred at rt for 1 h. The mixture was diluted with DCM, washed with 2 M aq. NaOH solution, 10% aq. Na₂S₂O₃ solution and then the organic phase was dried (MgSO₄) and concentrated in vacuo to afford Example 641C (5.7 g, 23.01 mmol, 90%) as an off-white solid, that was used without further purification. LRMS calculated for C₁₃H₁₀NO₂Cl: 247; found: 248 (M+H).

### Example 641D 1-[4-(benzyloxy)-2-chloropyridin-3-yl]prop-2-en-1-ol

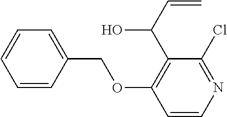 [SMILES: C=CC(O)c1c(OCc2ccccc2)ccnc1Cl]

To a solution of Example 641C (9.2 g, 37.15 mmol, 1 eq) in THE (200 mL), cooled to −78° C., was added vinylmagnesium bromide solution (55.7 mL, 1 M in THF, 55.7 mmol, 1.5 eq) dropwise. The mixture was stirred at −78° C. for 2 h and then at rt for 18 h. The mixture was cooled to 0° C., quenched by the dropwise addition of sat. aq. NH₄Cl solution and then extracted with EtOAc. The combined organic extracts were dried (MgSO₄) and concentrated in vacuo. Purification by automated flash chromatography (CombiFlash Rf, 120 g RediSep™ silica cartridge) eluting with a gradient of 0-50% EtOAc in heptane afforded Example 641D as a pale yellow solid (6.85 g, 24.84 mmol, 67%). LRMS calculated for C₁₅H₁₄NO₂Cl: 275; found: 276 (M+H). ¹H NMR (400 MHz, DMSO-d₆) δ ppm: 8.18 (d, J=5.7 Hz, 1H), 7.53-7.47 (m, 2H), 7.45-7.39 (m, 2H), 7.38-7.32 (m, 1H), 7.17 (d, J=5.7 Hz, 1H), 6.22 (ddd, J=17.3, 10.3, 5.9 Hz, 1H), 5.67 (tt, J=5.6, 1.6 Hz, 1H), 5.42 (d, J=5.3 Hz, 1H), 5.33-5.25 (m, 2H), 5.19 (dt, J=17.3, 1.7 Hz, 1H), 5.07 (dt, J=10.3, 1.6 Hz, 1H).

### Example 641E 1-[4-(benzyloxy)-2-chloropyridin-3-yl]prop-2-en-1-one

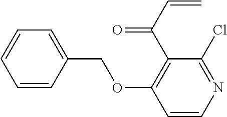 [SMILES: C=CC(=O)c1c(OCc2ccccc2)ccnc1Cl]

To a solution of Example 641D (6.8 g, 24.66 mmol, 1 eq) in DCM (120 mL), cooled to 0° C., was added DMP (10.99 g, 25.90 mmol, 1.05 eq) portionwise and then stirring continued at rt for 3 h. The mixture was diluted with DCM, washed with 2 M aq. NaOH solution, 1 M aq. Na₂S₂O₃ solution, dried (MgSO₄) and concentrated in vacuo to afford Example 641E as a yellow solid (6.4 g, 23.38 mmol, 95%). LRMS calculated for C₁₅H₁₂NO₂Cl: 273; found: 274 (M+H). ¹H NMR (400 MHz, DMSO-d₆) δ ppm: 8.40 (d, J=5.9 Hz, 1H), 7.46-7.30 (m, 6H), 6.60 (dd, J=17.5, 10.5 Hz, 1H), 6.31 (d, J=10.6 Hz, 1H), 6.03 (d, J=17.5 Hz, 1H), 5.29 (s, 2H).

### Example 641F 5-(benzyloxy)-1-[(4-methoxyphenyl)methyl]-2,3-dihydro-1,8-naphthyridin-4 (1H)-one

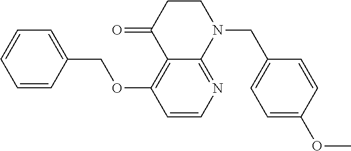 [SMILES: COc1ccc(CN2CCC(=O)c3c(OCc4ccccc4)ccnc32)cc1]

To a solution of Example 641E (2.4 g, 8.76 mmol, 1 eq) in DMF (42 mL) was added 4-methoxybenzylamine (1.49 mL, 11.4 mmol, 1.3 eq) and the reaction was stirred at 100° C. for 1.5 h under microwave irradiation. The reaction was allowed to cool to rt and concentrated in vacuo. Purification by automated flash chromatography (CombiFlash Rf, 40 g Gold RediSep™ silica cartridge) eluting with a gradient of 0-40% EtOAc in heptane afforded Example 641F as an oil (0.82 g, 2.18 mmol, 25%). LRMS calculated for C₂₃H₂₂N₂O₃: 374; found: 375 (M+H). ¹H NMR (400 MHz, CDCl₃) δ ppm: 8.11 (d, J=5.8 Hz, 1H), 7.57-7.52 (m, 2H), 7.42-7.36 (m, 2H), 7.33-7.27 (m, 1H), 7.24-7.19 (m, 2H), 6.87-6.82 (m, 2H), 6.25 (d, J=5.9 Hz, 1H), 5.24 (s, 2H), 4.89 (s, 2H), 3.79 (s, 3H), 3.47-3.40 (m, 2H), 2.67-2.60 (m, 2H).

### Example 641G 5-(benzyloxy)-1-[(4-methoxyphenyl)methyl]-4-methylidene-1,2,3,4-tetrahydro-1,8-naphthyridine

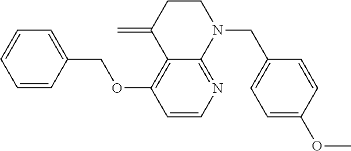 [SMILES: C=C1CCN(Cc2ccc(OC)cc2)c2nccc(OCc3ccccc3)c21]

LiHMDS (4.83 mL, 1 M in THF, 4.83 mmol, 1.6 eq) was added dropwise to a suspension of methyltriphenylphosphonium bromide (1.72 g, 4.83 mmol, 1.6 eq) in THE (16 mL), cooled to 0° C. under N₂ and then stirred for 20 min at 0° C. A solution of Example 641F (1.13 g, 3.02 mmol, 1 eq) in THE (8 mL) was added dropwise at 0° C. and the reaction was slowly allowed to warm to rt and stirred for 18 h. The mixture was cooled to 0° C., quenched with sat. aq. NaHCO₃ solution and then extracted with EtOAc, dried (MgSO₄) and concentrated in vacuo. Purification by automated flash chromatography (Combiflash Rf, Silica 40 g RediSep™ cartridge) eluting with a gradient of 0-40% EtOAc in heptane afforded Example 641G as an off-white powder (585 mg, 1.57 mmol, 52%). LRMS calculated for C₂₄H₂₄N₂O₂: 372; found: 373 (M+H). ¹H NMR (400 MHz, DMSO-d₆) δ ppm: 7.87 (d, J=5.8 Hz, 1H), 7.49-7.45 (m, 2H), 7.44-7.39 (m, 2H), 7.38-7.32 (m, 1H), 7.18-7.13 (m, 2H), 6.88-6.83 (m, 2H), 6.49 (d, J=5.8 Hz, 1H), 5.75 (d, J=2.6 Hz, 1H), 5.19 (s, 2H), 5.04-5.01 (m, 1H), 4.77 (s, 2H), 3.71 (s, 3H), 3.30-3.23 (m, 2H), 2.48-2.42 (m, 2H).

### Example 641H 8-[(4-methoxyphenyl)methyl]-5-methyl-5,6,7,8-tetrahydro-1,8-naphthyridin-4-ol, enantiomer 1

and

### Example 641I 8-[(4-methoxyphenyl)methyl]-5-methyl-5,6,7,8-tetrahydro-1,8-naphthyridin-4-ol, enantiomer 2

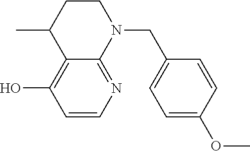 [SMILES: COc1ccc(CN2CCC(C)c3c(O)ccnc32)cc1]

To a solution of Example 641G (585 mg, 1.57 mmol, 1 eq) in EtOH (15 mL), MeOH (15 mL) and EtOAc (5 mL) was added catalytic amount of Pd/C under N₂. The mixture was evacuated and backfilled with N₂ (×3), then evacuated and backfilled with H₂ and shaken for 24 h at rt under an atmosphere of H₂. The reaction was filtered through celite, eluted with MeOH and concentrated in vacuo. Purification by automated flash chromatography (Combiflash Rf, Silica 24 g RediSep cartridge) eluting with a gradient of 0-20% MeOH in DCM afforded racemate. The enantiomers were separated by chiral chromatography. Column: OK, 50×500 mm, 20 μm. Eluents: iPrOH/Heptane 25:75+0.05% DEA. The enantiomer eluting earlier was collected as Example 641H (181 mg, 0.636 mmol, 41%). ¹H NMR (500 MHz, DMSO-d₆) δ ppm: 9.84 (br s, 1H), 7.61 (d, 1H), 7.13 (m, 2H), 6.85 (m, 2H), 6.09 (d, 1H), 4.76/4.69 (d+d, 2H), 3.71 (s, 3H), 3.26/3.13 (m+m, 2H), 3.09 (m, 1H), 1.72/1.59 (m+m, 2H), 1.05 (d, 3H). HRMS calculated for C₁₇H₂₀N₂O₂: 284.1525; found: 285.1593 (M+H).

The enantiomer eluting later was collected as Example 6411 (200 mg, 0.703 mmol, 45%). HRMS calculated for C₁₇H₂₀N₂O₂: 284.1525; found: 285.1592 (M+H).

### Example 641J methyl (1r,2′S,4S)-4-(3-chloroanilino)-2′-[(2R)-3-({8-[(4-methoxyphenyl)methyl]-5-methyl-5,6,7,8-tetrahydro-1,8-naphthyridin-4-yl}oxy)-2-methylpropyl]-2′,3′-dihydrospiro[cyclohexane-1,1′-indene]-4-carboxylate, diastereoisomer 1

and

### Example 642A methyl (1r,2′S,4S)-4-(3-chloroanilino)-2′-[(2R)-3-({8-[(4-methoxyphenyl)methyl]-5-methyl-5,6,7,8-tetrahydro-1,8-naphthyridin-4-yl}oxy)-2-methylpropyl]-2′,3′-dihydrospiro[cyclohexane-1,1′-indene]-4-carboxylate, diastereoisomer 2

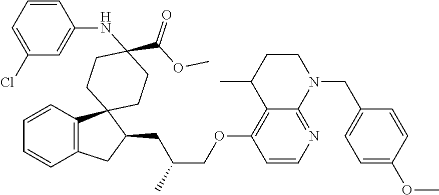 [SMILES: COc1ccc(CN2CCC(C)c3c(OC[C@H](C)C[C@H]4Cc5ccccc5[C@]45CC[C@@](Nc4cccc(Cl)c4)(C(=O)OC)CC5)ccnc32)cc1]

Using General procedure 30a and Preparation 10b1 (100 mg, 0.23 mmol, 1 eq) as the appropriate indane and Example 641H (97 mg, 0.34 mmol, 1.5 eq) as the appropriate alcohol, the mixture was heated at 70° C. for 1 h. The mixture was cooled to rt and partitioned between DCM and water. The organic phase was washed with brine, dried (MgSO₄) and concentrated in vacuo. Purification by automated flash chromatography (CombiFlash Rf, 24 g RediSep™ silica cartridge) eluting with a gradient of 0-37% EtOAc in heptane afforded Example 641J as a white gum that was used directly in the subsequent step without further purification. LRMS calculated for C₄₃H₅₀ClN₃O₄: 707; found: 708 (M+H).

Using General procedure 30a and Preparation 10b1 (100 mg, 0.23 mmol, 1 eq) as the appropriate indane and Example 6411 (96.5 mg, 0.34 mmol, 1.5 eq) as the appropriate alcohol, the mixture was heated at 70° C. for 2 h. The mixture was cooled to rt and partitioned between DCM and water. The organic phase was washed with brine, dried (MgSO₄) and concentrated in vacuo. Purification by automated flash chromatography (CombiFlash Rf, 24 g RediSep™ silica cartridge) eluting with a gradient of 0-50% EtOAc in heptane afforded Example 642A as a colourless gum that was used directly in the subsequent step without further purification. LRMS calculated for C₄₃H₅₀ClN₃O₄: 707; found: 708 (M+H).

### Example 641K methyl (1r,2′S,4S)-4-(3-chloroanilino)-2′-{(2R)-2-methyl-3-[(5-methyl-5,6,7,8-tetrahydro-1,8-naphthyridin-4-yl)oxy]propyl}-2′,3′-dihydrospiro[cyclohexane-1,1′-indene]-4-carboxylate, diastereoisomer 1

and

### Example 642B methyl (1r,2′S,4S)-4-(3-chloroanilino)-2′-{(2R)-2-methyl-3-[(5-methyl-5,6,7,8-tetrahydro-1,8-naphthyridin-4-yl)oxy]propyl}-2′,3′-dihydrospiro[cyclohexane-1,1′-indene]-4-carboxylate, diastereoisomer 2

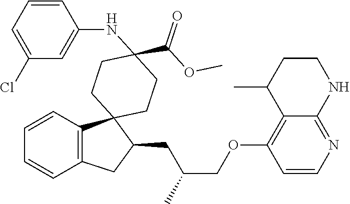 [SMILES: COC(=O)[C@]1(Nc2cccc(Cl)c2)CC[C@@]2(CC1)c1ccccc1C[C@@H]2C[C@@H](C)COc1ccnc2c1C(C)CCN2]

To a solution of Example 641J (194 mg, 0.27 mmol, 1 eq) in DCM (2 mL) was added TFA (0.5 mL) and the mixture was stirred at rt for 4 h. The mixture was diluted with water, basified with 2 M aq. NaOH solution, and then extracted with DCM. The organic phase was washed with brine, dried (MgSO₄) and concentrated in vacuo. Purification by automated flash chromatography (CombiFlash Rf, 12 g RediSep™ silica cartridge) eluting with a gradient of 0-23% MeOH in DCM afforded Example 641K as a yellow gum (92.5 mg, 0.16 mmol, 57%). LRMS calculated for C₃₅H₄₂ClN₃O₃: 587; found: 588 (M+H).

To a solution of Example 642A (220 mg, 0.31 mmol, 1 eq) in DCM (3 mL) was added TFA (0.5 mL) and the mixture was stirred at rt for 18 h. The mixture was diluted with water, basified with 2 M aq. NaOH solution and extracted with DCM. The organic phase was washed with brine, dried (MgSO₄) and concentrated in vacuo. Purification by automated flash chromatography (CombiFlash Rf, 12 g RediSep™ silica cartridge) eluting with a gradient of 0-17% MeOH in DCM afforded Example 642B as a yellow gum (110 mg, 0.19 mmol, 60%). LRMS calculated for C₃₅H₄₂ClN₃O₃: 587; found: 588 (M+H). ¹H NMR (400 MHz, DMSO-d₆) δ ppm: 7.77 (d, J=6.5 Hz, 1H), 7.42-7.36 (m, 1H), 7.25-7.12 (m, 3H), 7.06 (t, J=8.0 Hz, 1H), 6.98 (br s, 1H), 6.62-6.54 (m, 2H), 6.50-6.42 (m, 2H), 6.34 (s, 1H), 3.99-3.91 (m, 2H), 3.65 (s, 3H), 3.33-3.18 (m, 2H), 3.10-2.97 (m, 2H), 2.59-2.39 (m, 2H), 2.20-1.83 (m, 6H), 1.79-1.58 (m, 3H), 1.54-1.27 (m, 4H), 1.04 (d, J=6.6 Hz, 3H), 0.99 (d, J=6.9 Hz, 3H).

### Example 641 (1r,2′S,4S)-4-(3-chloroanilino)-2′-{(2R)-2-methyl-3-[(5-methyl-5,6,7,8-tetrahydro-1,8-naphthyridin-4-yl)oxy]propyl}-2′,3′-dihydrospiro[cyclohexane-1,1′-indene]-4-carboxylic acid, diastereoisomer 1

and

### Example 642 (1r,2′S,4S)-4-(3-chloroanilino)-2′-{(2R)-2-methyl-3-[(5-methyl-5,6,7,8-tetrahydro-1,8-naphthyridin-4-yl)oxy]propyl}-2′,3′-dihydrospiro[cyclohexane-1,1′-indene]-4-carboxylic acid, diastereoisomer 2

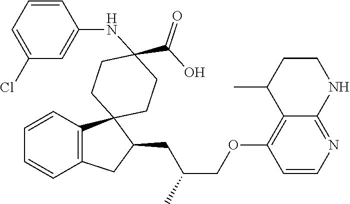 [SMILES: CC1CCNc2nccc(OC[C@H](C)C[C@H]3Cc4ccccc4[C@]34CC[C@@](Nc3cccc(Cl)c3)(C(=O)O)CC4)c21]

Using General procedure 33b and Example 641K (92.5 mg, 0.16 mmol, 1 eq) as the appropriate ester, Example 641 was obtained as a white powder (32.1 mg, 0.06 mmol, 36%). LRMS calculated for C₃₄H₄₀ClN₃O₃: 573; found: 574 (M+H). ¹H NMR (400 MHz, DMSO-d₆) δ ppm: 12.77 (br s, 1H), 7.74 (d, J=6.2 Hz, 1H), 7.41-7.34 (m, 1H), 7.24-7.12 (m, 3H), 7.06 (t, J=8.1 Hz, 1H), 6.77 (br s, 1H), 6.63 (t, J=2.2 Hz, 1H), 6.58-6.51 (m, 2H), 6.37 (d, J=6.2 Hz, 1H), 3.98-3.84 (m, 2H), 3.29-3.17 (m, 2H), 3.07-2.96 (m, 2H), 2.63-2.39 (m, 2H), 2.21-2.08 (m, 2H), 2.06-1.67 (m, 5H), 1.66-1.31 (m, 6H), 1.09 (d, J=7.0 Hz, 3H), 1.05 (d, J=6.6 Hz, 3H).

Using General procedure 33b and Example 642B (110 mg, 0.19 mmol, 1 eq) as the appropriate ester, Example 642 was obtained as a white powder (35.9 mg, 0.06 mmol, 33%). LRMS calculated for C₃₄H₄₀ClN₃O₃: 573; found: 574 (M+H). ¹H NMR (400 MHz, DMSO-d₆) δ ppm: 7.79 (d, J=6.6 Hz, 1H), 7.41-7.35 (m, 1H), 7.24-7.12 (m, 3H), 7.06 (t, J=8.1 Hz, 1H), 6.62 (t, J=2.1 Hz, 1H), 6.58-6.51 (m, 3H), 4.03-3.94 (m, 2H), 3.29-3.18 (m, 1H), 3.11-2.96 (m, 2H), 2.59-2.38 (m, 2H), 2.20-1.82 (m, 6H), 1.78-1.60 (m, 3H), 1.54-1.29 (m, 4H), 1.04 (d, J=6.6 Hz, 3H), 1.00 (d, J=7.0 Hz, 3H).

### Example 643 and Example 644

### Example 643A 5-(benzyloxy)-1-methyl-2,3-dihydro-1,8-naphthyridin-4 (1H)-one

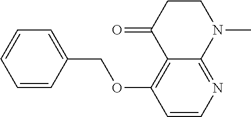 [SMILES: CN1CCC(=O)c2c(OCc3ccccc3)ccnc21]

To a solution of Example 641E (1.60 g, 5.84 mmol, 1 eq) in DMF (14 mL) was added MeNH₂ (4.38 mL, 2 M in THF, 1.5 eq) and the reaction was stirred at 100° C. for 1.5 h under microwave irradiation. The reaction was concentrated in vacuo and purification by automated flash chromatography (CombiFlash Rf, 40 g RediSep™ silica cartridge) eluting with a gradient of 0-20% EtOAc in DCM afforded Example 643A as an oil (790 mg, 2.94 mmol, 50%). LRMS calculated for C₁₆H₁₆N₂O₂: 268; found: 269 (M+H). ¹H NMR (400 MHz, CDCl₃) δ ppm: 8.11 (d, J=5.8 Hz, 1H), 7.56-7.51 (m, 2H), 7.42-7.36 (m, 2H), 7.33-7.27 (m, 1H), 6.24 (d, J=5.9 Hz, 1H), 5.23 (s, 2H), 3.55-3.49 (m, 2H), 3.19 (s, 3H), 2.74-2.68 (m, 2H).

### Example 643B 5-(benzyloxy)-1-methyl-4-methylidene-1,2,3,4-tetrahydro-1,8-naphthyridine

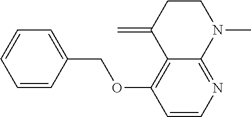 [SMILES: C=C1CCN(C)c2nccc(OCc3ccccc3)c21]

To a suspension of methyltriphenylphosphonium bromide (2.21 g, 6.22 mmol, 1.6 eq) in THE (20 mL) cooled to 0° C., was added LiHMDS (6.2 mL, 1 M in THF, 6.2 mmol, 1.6 eq) dropwise and the mixture stirred for 20 min. A solution of Example 643A (1.045 g, 3.89 mmol, 1 eq) in THE (10 mL) was added dropwise and the reaction was slowly allowed to warm to rt and stirred for 1 h. The mixture was cooled in an ice bath, quenched by the careful addition of sat. aq. NaHCO₃ solution and extracted with EtOAc. The combined organic extracts were washed with brine, dried (MgSO₄) and concentrated in vacuo. Purification by automated flash chromatography (CombiFlash Rf, 40 g RediSep™ silica cartridge) eluting with a gradient of 0-60% EtOAc in heptane afforded Example 643B as a white solid (0.59 g, 2.2 mmol, 56%). LRMS calculated for C₁₇H₁₈N₂O: 266; found: 267 (M+H). ¹H NMR (400 MHz, CDCl₃) δ ppm: 7.91 (d, J=5.8 Hz, 1H), 7.44-7.30 (m, 5H), 6.27 (d, J=5.8 Hz, 1H), 5.86-5.82 (m, 1H), 5.13 (s, 2H), 5.10-5.05 (m, 1H), 3.41-3.35 (m, 2H), 3.13 (s, 3H), 2.62-2.55 (m, 2H).

### Example 643C 5,8-dimethyl-5,6,7,8-tetrahydro-1,8-naphthyridin-4-ol, enantiomer 1

and

### Example 643D 5,8-dimethyl-5,6,7,8-tetrahydro-1,8-naphthyridin-4-ol, enantiomer 2

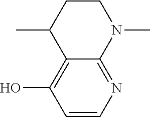

To a solution of Example 643B (550 mg, 2.07 mmol, 1 eq) in EtOH (30 mL) was added catalytic amount of Pd/C under N₂. The mixture was evacuated and backfilled with N₂ (×3), then evacuated and backfilled with H₂ and shaken for 18 h at rt under an atmosphere of H₂. The reaction was filtered through celite, eluted with MeOH and concentrated in vacuo. Purification by automated flash chromatography (Combiflash Rf, Silica 12 g RediSep cartridge) eluting with a gradient of 0-20% MeOH in DCM afforded racemate. The enantiomers were separated by chiral chromatography. Column: IC, 50×500 mm, 20 μm. Eluents: iPrOH/Heptane 50:50+0.05% DEA. The enantiomer eluting earlier was collected as Example 643C. ¹H NMR (500 MHz, DMSO-d₆) δ ppm: 9.90 (br s, 1H), 7.59 (d, 1H), 6.07 (d, 1H), 3.32/3.17 (m+m, 2H), 3.05 (m, 1H), 2.99 (s, 3H), 1.79/1.60 (m+m, 2H), 1.07 (d, 3H). HRMS calculated for C₁₀H₁₄N₂O: 178.1106; found: 179.1178 (M+H).

The enantiomer eluting later was collected as Example 643D. HRMS calculated for C₁₀H₁₄N₂O: 178.1106; found: 179.1186 (M+H).

### Example 643 (1r,2′S,4S)-4-(3-chloroanilino)-2′-{(2R)-3-[(5,8-dimethyl-5,6,7,8-tetrahydro-1,8-naphthyridin-4-yl)oxy]-2-methylpropyl}-2′,3′-dihydrospiro[cyclohexane-1,1′-indene]-4-carboxylic acid, diastereoisomer 1

and

### Example 644 (1r,2′S,4S)-4-(3-chloroanilino)-2′-{(2R)-3-[(5,8-dimethyl-5,6,7,8-tetrahydro-1,8-naphthyridin-4-yl)oxy]-2-methylpropyl}-2′,3′-dihydrospiro[cyclohexane-1,1′-indene]-4-carboxylic acid, diastereoisomer 2

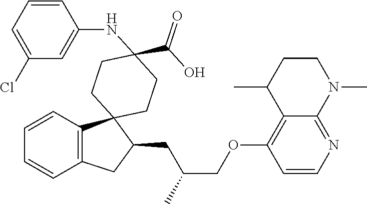 [SMILES: CC1CCN(C)c2nccc(OC[C@H](C)C[C@H]3Cc4ccccc4[C@]34CC[C@@](Nc3cccc(Cl)c3)(C(=O)O)CC4)c21]

Using General procedure 30a and Preparation 10b1 (150 mg, 0.34 mmol, 1 eq) as the appropriate indane and Example 643C (115 mg, 0.64 mmol, 1.9 eq) as the appropriate alcohol, afforded an intermediate which was hydrolyzed according to General procedure 33b to afford Example 643 as a white powder (40.9 mg, 0.07 mmol, 21%). LRMS calculated for C₃₅H₄₂ClN₃O₃: 587; found: 588 (M+H). ¹H NMR (400 MHz, DMSO-d₆) δ ppm: 7.79 (d, J=5.8 Hz, 1H), 7.41-7.33 (m, 1H), 7.24-7.11 (m, 3H), 7.06 (t, J=8.1 Hz, 1H), 6.63 (t, J=2.1 Hz, 1H), 6.57-6.51 (m, 2H), 6.26 (d, J=5.8 Hz, 1H), 3.88-3.77 (m, 2H), 3.35-3.26 (m, 1H), 3.22-3.14 (m, 1H), 3.08-2.96 (m, 5H), 2.60-2.40 (m, 2H), 2.19-2.08 (m, 2H), 2.05-1.81 (m, 4H), 1.80-1.69 (m, 2H), 1.62-1.55 (m, 1H), 1.53-1.31 (m, 4H), 1.08 (d, J=6.9 Hz, 3H), 1.04 (d, J=6.7 Hz, 3H).

Using General procedure 30a and Preparation 10b1 (100 mg, 0.23 mmol, 1 eq) as the appropriate indane and Example 643D (60.5 mg, 0.34 mmol, 1.5 eq) as the appropriate alcohol, afforded an intermediate which was hydrolyzed according to General procedure 33b to afford Example 644 as a white powder (24.7 mg, 0.04 mmol, 38%). LRMS calculated for C₃₅H₄₂ClN₃O₃: 587; found: 588 (M+H). ¹H NMR (400 MHz, DMSO-d₆) δ ppm: 7.82 (d, J=6.5 Hz, 1H), 7.42-7.34 (m, 1H), 7.25-7.12 (m, 3H), 7.06 (t, J=8.1 Hz, 1H), 6.62 (t, J=2.1 Hz, 1H), 6.59-6.49 (m, 3H), 4.04-3.88 (m, 2H), 3.46-3.22 (m, 2H), 3.13-2.96 (m, 5H), 2.59-2.39 (m, 2H), 2.20-2.07 (m, 2H), 2.06-1.59 (m, 7H), 1.54-1.28 (m, 4H), 1.04 (d, J=6.7 Hz, 3H), 0.98 (d, J=6.9 Hz, 3H).

### Example 645 (1r,4R)-4-(3-chloroanilino)-2′-{(2R)-2-methyl-3-[(5,6,7,8-tetrahydroquinolin-4-yl)oxy]propyl}-2′,3′-dihydrospiro[cyclohexane-1,1′-indene]-4-carboxylic acid

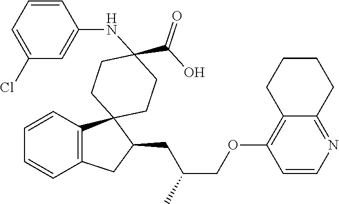 [SMILES: C[C@@H](COc1ccnc2c1CCCC2)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and 4-chloro-5,6,7,8-tetrahydroquinoline (131 mg, 0.78 mmol, 2.3 eq) as the appropriate aryl chloride, Example 645 was obtained as a beige powder (14.7 mg, 0.03 mmol, 8%). LRMS calculated for C₃₄H₃₉ClN₂O₃: 558; found: 559 (M+H). ¹H NMR (400 MHz, DMSO-d₆) δ ppm: 8.15 (d, J=5.6 Hz, 1H), 7.42-7.35 (m, 1H), 7.23-7.11 (m, 3H), 7.08-7.00 (m, 1H), 6.77 (d, J=5.6 Hz, 1H), 6.66-6.59 (m, 1H), 6.59-6.50 (m, 2H), 3.88 (d, J=6.0 Hz, 2H), 3.00 (dd, J=15.4, 7.0 Hz, 1H), 2.76-2.70 (m, 2H), 2.60-2.39 (m, 4H), 2.18-2.06 (m, 2H), 2.06-1.80 (m, 4H), 1.80-1.62 (m, 5H), 1.54-1.30 (m, 4H), 1.04 (d, J=6.6 Hz, 3H).

### Example 646 (1r,4R)-4-(3-chloroanilino)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

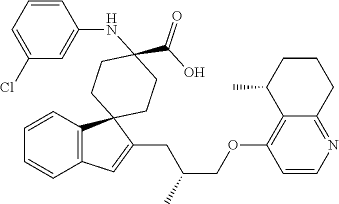 [SMILES: C[C@@H](COc1ccnc2c1[C@H](C)CCC2)CC1=Cc2ccccc2[C@]12CC[C@@](Nc1cccc(Cl)c1)(C(=O)O)CC2]

Using General procedure 32 and Preparation 10a as the appropriate indene and Preparation 2a1 as the appropriate alcohol Example 646 was obtained. HRMS calculated for C₃₅H₃₉N₂O₃Cl: 570.2649; found: 571.2715 (M+H).

### Example 647 (1r,4R)-4-(3-chloroanilino)-2′-[(2R)-2-methyl-3-{[(5S)-5-methyl-5,6,7,8-tetrahydroquinolin-4-yl]oxy}propyl]spiro[cyclohexane-1,1′-indene]-4-carboxylic acid

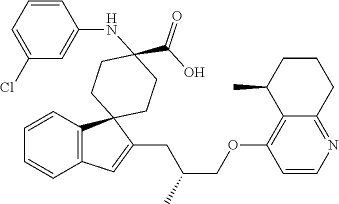 [SMILES: C[C@@H](COc1ccnc2c1[C@@H](C)CCC2)CC1=Cc2ccccc2[C@]12CC[C@@](Nc1cccc(Cl)c1)(C(=O)O)CC2]

Using General procedure 32 and Preparation 10a as the appropriate indene and Preparation 2a2 as the appropriate alcohol Example 647 was obtained. HRMS calculated for C₃₅H₃₉N₂O₃Cl: 570.2649; found: 571.2715 (M+H).

### Example 648

### Example 648A 5,6,7,8-tetrahydro-5,8-ethanoquinoline

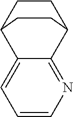 [SMILES: c1cnc2c(c1)C1CCC2CC1]

To a solution of bicyclo[2.2.2]octan-2-one (746 mg, 6.01 mmol, 1 eq) in EtOH (20 mL) was added prop-2-yn-1-amine (827 μL, 12.01 mmol, 2 eq) and NaAuCl₄×2H₂O (72 mg, 0.18 mmol, 0.03 eq). The mixture was heated in a sealed tube at 80° C. for 5 h. The mixture was cooled to rt, filtered through celite and concentrated in vacuo. Purification by automated flash chromatography (CombiFlash Rf, Silica 12 g RediSep™ cartridge) eluting with a gradient of 0-50% EtOAc in heptane afforded Example 648A as a brown oil (86.5 mg, 0.54 mmol, 9%). LRMS calculated for C₁₁H₁₃N: 159; found: 160 (M+H). ¹H NMR (400 MHz, DMSO-d₆) δ ppm: 8.27 (dd, J=5.1, 1.7 Hz, 1H), 7.54-7.50 (m, 1H), 7.18 (dd, J=7.4, 5.1 Hz, 1H), 3.11-3.03 (m, 2H), 1.88-1.73 (m, 4H), 1.42-1.24 (m, 4H).

### Example 648B 1-oxo-5,6,7,8-tetrahydro-5,8-ethano-1λ⁵-quinoline

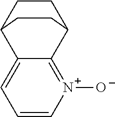 [SMILES: [O-][n+]1cccc2c1C1CCC2CC1]

To a solution of Example 648A (367 mg, 2.3 mmol, 1 eq) in DCM (10 mL), cooled to 0° C., was added mCPBA (77% purity, 775 mg, 3.46 mmol, 1.5 eq) portionwise and the mixture was slowly allowed to warm to rt and stirred for 3 h. The mixture was cooled to 0° C., sat. aq. NaHCO₃ solution was added slowly, and the mixture was stirred for 30 min. Then it was partitioned between DCM and 2 M aq. NaOH solution/sat. aq. NaHCO₃ solution (1:1). The phases were separated, and the organic phase was washed with brine, dried (MgSO₄) and concentrated in vacuo to afford Example 648B as a yellow oil that was used directly in the subsequent step without further purification. LRMS calculated for C₁₁H₁₃NO: 175; found: 176 (M+H). ¹H NMR (400 MHz, DMSO-d₆) δ ppm: 8.12-8.07 (m, 1H), 7.23-7.16 (m, 2H), 3.92-3.87 (m, 1H), 3.17-3.12 (m, 1H), 1.89-1.70 (m, 4H), 1.40-1.22 (m, 4H).

### Example 648C 4-chloro-5,6,7,8-tetrahydro-5,8-ethanoquinoline

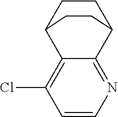 [SMILES: Clc1ccnc2c1C1CCC2CC1]

A solution of Example 648B (439 mg, 2.51 mmol, 1 eq) in POCl₃ (1.87 mL, 20 mmol, 8 eq) was heated at 90° C. for 18 h. The mixture was cooled to rt and added dropwise to a rapidly stirring ice-water and stirred for 30 min. The mixture was basified with aq. NH₃ solution and then extracted with DCM. The organic phase was washed with brine, dried (MgSO₄) and concentrated in vacuo. Purification by automated flash chromatography (CombiFlash Rf, Silica 12 g RediSep™ cartridge) eluting with a gradient of 0-22% EtOAc in heptane afforded Example 648C as a brown oil (290 mg, 1.5 mmol, 60%). LRMS calculated for C₁₁H₁₂ClN: 193; found: 194 (M+H). ¹H NMR (400 MHz, DMSO-d₆) δ ppm: 8.25 (d, J=5.6 Hz, 1H), 7.36 (d, J=5.5 Hz, 1H), 3.48-3.44 (m, 1H), 3.15-3.10 (m, 1H), 1.90-1.78 (m, 4H), 1.43-1.24 (m, 4H).

### Example 648 (1r,2′S,4S)-4-(3-chloroanilino)-2′-{(2R)-2-methyl-3-[(5,6,7,8-tetrahydro-5,8-ethanoquinolin-4-yl)oxy]propyl}-2′,3′-dihydrospiro[cyclohexane-1,1′-indene]-4-carboxylic acid

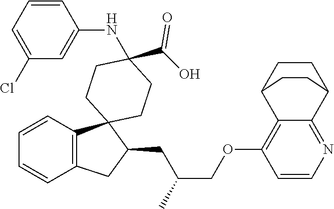 [SMILES: C[C@@H](COc1ccnc2c1C1CCC2CC1)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and Example 648C (151 mg, 0.78 mmol, 2.3 eq) as the appropriate aryl chloride, Example 648 was obtained as a white powder (98 mg, 0.17 mmol, 49%). LRMS calculated for C₃₆H₄₁ClN₂O₃: 584; found: 585 (M+H). ¹H NMR (400 MHz, DMSO-d₆) δ ppm: 8.48 (d, J=6.7 Hz, 1H), 7.41-7.32 (m, 2H), 7.23-7.11 (m, 3H), 7.06 (t, J=8.1 Hz, 1H), 6.63 (t, J=2.1 Hz, 1H), 6.59-6.51 (m, 2H), 4.14 (d, J=6.1 Hz, 2H), 3.47-3.40 (m, 1H), 3.31-3.26 (m, 1H), 3.01 (dd, J=15.6, 7.1 Hz, 1H), 2.60-2.39 (m, 2H), 2.21-1.66 (m, 11H), 1.54-1.30 (m, 6H), 1.27-1.09 (m, 2H), 1.06 (d, J=6.6 Hz, 3H).

### Example 649

### Example 649A 3-methyl-1-oxo-5,6,7,8-tetrahydro-1λ⁵-quinoline

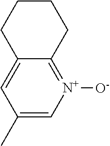

To a solution of 3-methyl-5,6,7,8-tetrahydroquinoline (500 mg, 3.4 mmol, 1 eq) in DCM (10 mL), cooled to 0° C., was added mCPBA (77% purity, 0.91 g, 4.08 mmol, 1.2 eq) portionwise and the mixture was warmed slowly to rt and stirred for 18 h. The mixture was cooled to 0° C., sat. aq. NaHCO₃ solution was added slowly and then stirred for 30 min. The mixture was partitioned between DCM and 2 M aq. NaOH solution. The phases were separated, and the organic phase was washed brine, dried (MgSO₄) and concentrated in vacuo. Purification by automated flash chromatography (CombiFlash Rf, Silica 12 g RediSep™ cartridge) eluting with a gradient of 0-10% MeOH in DCM afforded Example 649A as a colourless oil. LRMS calculated for C₁₀H₁₃NO: 163; found: 164 (M+H). ¹H NMR (400 MHz, DMSO-d₆) δ ppm: 8.03-8.00 (m, 1H), 6.96-6.92 (m, 1H), 2.72-2.64 (m, 4H), 2.21-2.17 (m, 3H), 1.83-1.74 (m, 2H), 1.70-1.62 (m, 2H).

### Example 649B 4-chloro-3-methyl-5,6,7,8-tetrahydroquinoline

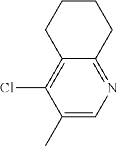

A solution of Example 649A (605 mg, 3.71 mmol, 1 eq) in POCl₃ (2.76 mL, 29.7 mmol, 8 eq) was heated at 80° C. for 18 h. A mixture of regioisomers was formed. The mixture was cooled to rt and added dropwise to a rapidly stirring ice-water and stirred for 30 min. The mixture was basified with aq. NH₃ solution and extracted with DCM. The organic phase was washed with brine, dried (MgSO₄) and concentrated in vacuo. The regioisomers were separated and purified by automated flash chromatography (CombiFlash Rf, Silica 12 g RediSep™ cartridge) eluting with a gradient of 0-33% EtOAc in heptane. The regioisomer eluting later was collected as Example 649B, isolated as a colourless oil (411 mg, 2.26 mmol, 61%). LRMS calculated for C₁₀H₁₂ClN: 181; found: 182 (M+H). ¹H NMR (400 MHz, DMSO-d₆) δ ppm: 8.27-8.24 (m, 1H), 2.87-2.66 (m, 4H), 2.30-2.26 (m, 3H), 1.83-1.73 (m, 4H).

### Example 649 (1r,2′S,4S)-4-(3-chloroanilino)-2′-{(2R)-2-methyl-3-[(3-methyl-5,6,7,8-tetrahydroquinolin-4-yl)oxy]propyl}-2′,3′-dihydrospiro[cyclohexane-1,1′-indene]-4-carboxylic acid

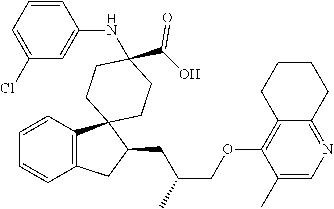 [SMILES: Cc1cnc2c(c1OC[C@H](C)C[C@H]1Cc3ccccc3[C@]13CC[C@@](Nc1cccc(Cl)c1)(C(=O)O)CC3)CCCC2]

Using General procedure 31c and Preparation 10b1 (100 mg, 0.23 mmol, 1 eq) as the appropriate alcohol and Example 649B (86.3 mg, 0.48 mmol, 2.1 eq) as the appropriate aryl chloride, Example 649 was obtained as a beige powder (76.3 mg, 0.13 mmol, 55%). LRMS calculated for C₃₅H₄₁ClN₂O₃: 572; found: 573 (M+H). ¹H NMR (400 MHz, DMSO-d₆) δ ppm: 8.45-8.38 (m, 1H), 7.43-7.35 (m, 1H), 7.26-7.12 (m, 3H), 7.06 (t, J=8.1 Hz, 1H), 6.63 (t, J=2.1 Hz, 1H), 6.59-6.50 (m, 2H), 4.05 (d, J=5.6 Hz, 2H), 3.00 (dd, J=15.6, 7.1 Hz, 1H), 2.92-2.84 (m, 2H), 2.75-2.64 (m, 2H), 2.60-2.37 (m, 2H), 2.32-2.27 (m, 3H), 2.21-2.08 (m, 2H), 2.07-1.84 (m, 4H), 1.83-1.64 (m, 5H), 1.56-1.45 (m, 2H), 1.45-1.33 (m, 2H), 1.07 (d, J=6.7 Hz, 3H).

### Example 650

### Example 650A 7-methyl-5,6,7,8-tetrahydroquinoline

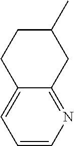

PtO₂ (119 mg, 0.52 mmol, 0.05 eq) was added to a solution of 7-methylquinoline (1.5 g, 10.48 mmol, 1 eq) in TFA (35 mL, 469.64 mmol, 45 eq). The mixture was evacuated and backfilled with N₂ (×3), then evacuated and backfilled with H₂ and shaken at 50° C. under an atmosphere of H₂ for 4 h. The mixture was allowed to cool to rt and filtered through celite, washed with minimal MeOH. The solvents were removed in vacuo, and the residue was dissolved in DCM and washed with sat. aq. NaHCO₃ solution. The organic phase was dried (MgSO₄) and concentrated in vacuo. Purification by automated flash chromatography (Combiflash Rf, Silica 24 g RediSep™ cartridge) eluting with a gradient of 0-44% EtOAc (3% TEA) in heptane followed by reverse phase automated flash chromatography (Combiflash Rf, C18 50 g Gold, RediSep column) eluting with a gradient of 10-60% MeCN in water afforded a racemate, Example 650A, isolated as a colourless oil (444 mg, 3.02 mmol, 29%). LRMS calculated for C₁₀H₁₃N: 147; found: 148 (M+H). ¹H NMR (400 MHz, DMSO-d₆) δ ppm: 8.31-8.25 (m, 1H), 7.49-7.43 (m, 1H), 7.10 (dd, J=7.7, 4.7 Hz, 1H), 2.94-2.84 (m, 1H), 2.82-2.70 (m, 2H), 2.47-2.37 (m, 1H), 1.96-1.80 (m, 2H), 1.42-1.28 (m, 1H), 1.05 (d, J=6.6 Hz, 3H).

### Example 650B 7-methyl-1-oxo-5,6,7,8-tetrahydro-1λ⁵-quinoline

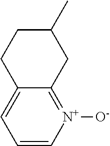

mCPBA (70% purity, 892 mg, 3.62 mmol, 1.2 eq) was added portionwise to a stirred solution of Example 650A (444 mg, 3.02 mmol, 1 eq) in DCM (15 mL) and the mixture was stirred at rt for 18 h. 2 M aq. NaOH solution was added and the mixture was stirred for 30 min and then diluted with DCM, dried (PTFE phase separator) and concentrated in vacuo to afford a racemate, Example 650B, isolated as a white powder (436 mg, 2.67 mmol, 89%). LRMS calculated for C₁₀H₁₃NO: 163; found: 164 (M+H). ¹H NMR (400 MHz, DMSO-d₆) δ ppm: 8.14-8.09 (m, 1H), 7.22-7.15 (m, 1H), 7.13-7.07 (m, 1H), 3.07-2.97 (m, 1H), 2.86-2.70 (m, 2H), 2.19-2.07 (m, 1H), 1.91-1.75 (m, 2H), 1.35-1.22 (m, 1H), 1.08 (d, J=6.5 Hz, 3H).

### Example 650C 4-chloro-7-methyl-5,6,7,8-tetrahydroquinoline

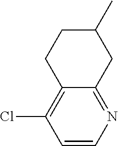

POCl₃ (2.48 mL, 26.59 mmol, 10 eq) was added slowly to Example 650B (434 mg, 2.66 mmol, 1 eq) cooled to −5° C. The mixture was allowed to warm to rt and then heated at 80° C. for 5 h.

The mixture was allowed to cool to rt and added dropwise to vigorously stirred ice-water, stirred for 30 min and then basified by the slow addition of aq. NH₃ solution. The mixture was extracted with DCM and the combined organic extracts dried (MgSO₄) and concentrated in vacuo. Purification by automated flash chromatography (Combiflash Rf, Silica 12 g RediSep™ cartridge) eluting with a gradient of 0-35% EtOAc (3% TEA) in heptane afforded a racemate, Example 650C, isolated as a colourless oil (343 mg, 1.89 mmol, 71%). LRMS calculated for C₁₀H₁₂ClN: 181; found: 182 (M+H). ¹H NMR (400 MHz, DMSO-d₆) δ ppm: 8.30-8.24 (m, 1H), 7.33 (d, J=5.2 Hz, 1H), 2.97-2.81 (m, 2H), 2.73-2.60 (m, 1H), 2.51-2.42 (m, 1H), 1.96-1.82 (m, 2H), 1.45-1.32 (m, 1H), 1.05 (d, J=6.4 Hz, 3H).

### Example 650 (1r,2′S,4S)-4-(3-chloroanilino)-2′-{(2R)-2-methyl-3-[(7-methyl-5,6,7,8-tetrahydroquinolin-4-yl)oxy]propyl}-2′,3′-dihydrospiro[cyclohexane-1,1′-indene]-4-carboxylic acid

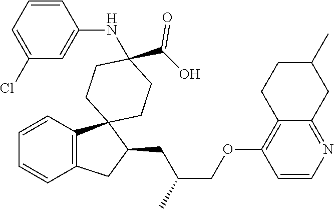 [SMILES: CC1CCc2c(OC[C@H](C)C[C@H]3Cc4ccccc4[C@]34CC[C@@](Nc3cccc(Cl)c3)(C(=O)O)CC4)ccnc2C1]

Using General procedure 33b and Preparation 10b1 as the appropriate indane, followed by General Procedure 31c and Example 650C as the appropriate aryl chloride, Example 650 was obtained as a mixture of diastereoisomers. ¹H NMR (500 MHz, DMSO-d₆) δ ppm: 12.73 (br s, 1H), 8.14 (d, 1H), 7.37-7.13 (m, 4H), 7.04 (t, 1H), 6.76/6.75 (d/d, 1H), 6.61 (t, 1H), 6.54 (dd, 1H), 6.54 (dd, 1H), 6.26 (br s, 1H), 3.87 (m, 2H), 2.99/2.54 (dd+dd, 2H), 2.80/2.35 (m+m, 2H), 2.69/2.42 (m+m, 2H), 2.42-1.38 (m, 8H), 2.12 (m, 1H), 1.98 (m, 1H), 1.81 (m, 1H), 1.80/1.23 (m+m, 2H), 1.42/1.35 (m+m, 2H), 1.03 (d, 3H), 1.02 (d, 3H). HRMS calculated for C₃₅H₄₁N₂O₃Cl: 572.2806; found: 573.2875 (M+H).

### Example 651

### Example 651A 6-methyl-5,6,7,8-tetrahydroquinoline

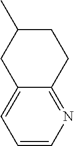

To a solution of 4-methylcyclohexanone 1 (0.74 mL, 6 mmol, 1 eq) in EtOH (20 mL) was added prop-2-yn-1-amine (0.83 mL, 12 mmol, 2 eq) and NaAuCl₄×2H₂O (72 mg, 0.18 mmol, 0.03 eq) and the mixture was heated at 80° C. for 5 h in a sealed tube. The mixture was allowed to cool to rt, filtered through celite, washed with EtOH and the filtrate was concentrated in vacuo. Purification by automated flash chromatography (CombiFlash Rf, Silica 12 g RediSep™ cartridge) eluting with a gradient of 0-50% EtOAc (3% TEA) in heptane afforded a racemate, Example 651A, isolated as a yellow oil (465 mg, 3.16 mmol, 53%). ¹H NMR (400 MHz, CDCl₃) δ ppm: 8.37-8.32 (m, 1H), 7.35-7.30 (m, 1H), 7.04-6.98 (m, 1H), 3.05-2.86 (m, 2H), 2.84-2.75 (m, 1H), 2.47-2.36 (m, 1H), 2.02-1.82 (m, 2H), 1.57-1.44 (m, 1H), 1.08 (d, J=6.5 Hz, 3H).

### Example 651B 6-methyl-1-oxo-5,6,7,8-tetrahydro-1λ⁵-quinoline

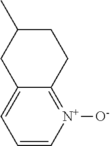

To a solution of Example 651A (465 mg, 3.16 mmol, 1 eq) in DCM (10 mL) was added mCPBA (70% purity, 934 mg, 3.79 mmol, 1.2 eq) portionwise and the mixture was stirred at rt for 2 h. 1 M aq. NaOH solution was added slowly and the mixture was stirred at rt for 30 min, then partitioned between DCM and water. The organic phase was washed with brine, dried (PTFE phase separator) and concentrated in vacuo to afford a racemate, Example 651B, isolated as a yellow solid (467 mg, 2.86 mmol, 91%). LRMS calculated for C₁₀H₁₃NO: 163; found: 164 (M+H). ¹H NMR (400 MHz, DMSO-d₆) δ ppm: 8.13-8.09 (m, 1H), 7.22-7.16 (m, 1H), 7.10-7.06 (m, 1H), 2.97-2.88 (m, 1H), 2.85-2.76 (m, 1H), 2.64-2.52 (m, 1H), 2.44-2.33 (m, 1H), 1.98-1.89 (m, 1H), 1.84-1.70 (m, 1H), 1.45-1.32 (m, 1H), 1.02 (d, J=6.6 Hz, 3H).

### Example 651C 4-chloro-6-methyl-5,6,7,8-tetrahydroquinoline

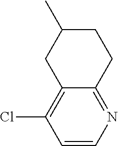

A solution of Example 651B (647 mg, 3.96 mmol, 1 eq) in POCl₃ (3.69 mL, 39.6 mmol, 10 eq) was heated at 90° C. for 24 h. The mixture was cooled to rt and added dropwise to rapidly stirring ice-water and stirred for 30 min. The mixture was basified with aq. NH₃ solution and extracted with DCM. The organic phase was washed with brine, dried (MgSO₄) and concentrated in vacuo. Purification by automated flash chromatography (CombiFlash Rf, Silica 12 g RediSep™ cartridge) eluting with a gradient of 0-27% EtOAc (3% TEA) in heptane afforded a racemate, Example 651C, isolated as a colourless oil (259 mg, 1.43 mmol, 36%). LRMS calculated for C₁₀H₁₂ClN: 181; found: 182 (M+H). ¹H NMR (400 MHz, DMSO-d₆) δ ppm: 8.29-8.25 (m, 1H), 7.35-7.30 (m, 1H), 2.97-2.85 (m, 3H), 2.31-2.20 (m, 1H), 1.94-1.79 (m, 2H), 1.50-1.37 (m, 1H), 1.08 (d, J=6.5 Hz, 3H).

### Example 651 (1r,2′S,4S)-4-(3-chloroanilino)-2′-{(2R)-2-methyl-3-[(6-methyl-5,6,7,8-tetrahydroquinolin-4-yl)oxy]propyl}-2′,3′-dihydrospiro[cyclohexane-1,1′-indene]-4-carboxylic acid

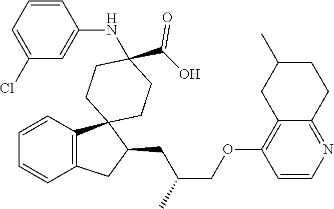 [SMILES: CC1CCc2nccc(OC[C@H](C)C[C@H]3Cc4ccccc4[C@]34CC[C@@](Nc3cccc(Cl)c3)(C(=O)O)CC4)c2C1]

Using General procedure 31c and Preparation 10b1 (150 mg, 0.34 mmol, 1 eq) as the appropriate alcohol and Example 651C (136 mg, 0.75 mmol, 2.2 eq) as the appropriate aryl chloride, Example 651 was obtained as a mixture of diastereoisomers, isolated as a white powder (138 mg, 0.24 mmol, 71%). LRMS calculated for C₃₅H₄₁ClN₂O₃: 572; found: 573 (M+H). ¹H NMR (400 MHz, DMSO-d₆) δ ppm: 8.16 (d, J=5.6 Hz, 1H), 7.42-7.32 (m, 1H), 7.24-7.11 (m, 3H), 7.05 (t, J=8.1 Hz, 1H), 6.81-6.77 (m, 1H), 6.63 (t, J=2.1 Hz, 1H), 6.58-6.52 (m, 2H), 3.94-3.82 (m, 2H), 3.00 (dd, J=15.6, 7.1 Hz, 1H), 2.82-2.66 (m, 3H), 2.59-2.38 (m, 2H), 2.21-2.09 (m, 2H), 2.07-1.65 (m, 8H), 1.54-1.26 (m, 5H), 1.08-0.96 (m, 6H).

### Example 652

### Example 652A 6,6-dimethyl-5,6,7,8-tetrahydroquinoline

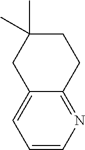

To a solution of 4,4-dimethylcyclohexanone (1.08 g, 8.56 mmol, 1.08 eq) in EtOH (20 mL) was added prop-2-yn-1-amine (1.09 mL, 15.9 mmol, 2 eq) and NaAuCl₄×2H₂O (63 mg, 0.16 mmol, 0.02 eq). The mixture was heated at 80° C. for 18 h in a sealed tube. The mixture was allowed to cool to rt, filtered through celite, washed with EtOH. The filtrate was concentrated in vacuo. Purification by automated flash chromatography (CombiFlash Rf, Silica 24 g RediSep™ cartridge) eluting with a gradient of 0-37% EtOAc (3% TEA) in heptane afforded Example 652A as a yellow oil (1.05 g, 6.51 mmol, 76%). LRMS calculated for C_{II}H₁₅N: 161; found: 162 (M+H). ¹H NMR (400 MHz, CDCl₃) δ ppm: 8.39-8.33 (m, 1H), 7.35-7.29 (m, 1H), 7.05-6.99 (m, 1H), 2.99-2.91 (m, 2H), 2.54 (s, 2H), 1.70-1.65 (m, 2H), 1.01 (s, 6H).

### Example 652B 6,6-dimethyl-1-oxo-5,6,7,8-tetrahydro-1λ⁵-quinoline

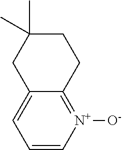

To a solution of Example 652A (1 g, 6.2 mmol, 1 eq) in DCM (30 mL) was added mCPBA (75% purity, 1.71 g, 7.44 mmol, 1.2 eq) portionwise and the mixture was stirred at rt for 3 h. 2 M aq. NaOH solution (30 mL) was added slowly and the mixture was stirred at rt for 30 min, and then partitioned between DCM and water. The organic phase was washed with brine, dried (MgSO₄) and concentrated in vacuo to afford Example 652B as an orange solid that was used directly in the subsequent step without further purification. LRMS calculated for C₁₁H₁₅NO: 177; found: 178 (M+H). ¹H NMR (400 MHz, DMSO-d₆) δ ppm: 8.14-8.09 (m, 1H), 7.23-7.17 (m, 1H), 7.10-7.04 (m, 1H), 2.72 (t, J=6.8 Hz, 2H), 2.55 (s, 2H), 1.61 (t, J=6.8 Hz, 2H), 0.94 (s, 6H).

### Example 652C 4-chloro-6,6-dimethyl-5,6,7,8-tetrahydroquinoline

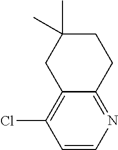

A solution of Example 652B (1 g, 5.64 mmol, 1 eq) in POCl₃ (5.26 mL, 56.4 mmol, 10 eq) was heated at 90° C. for 3 h. The mixture was cooled to rt and added dropwise to a rapidly stirring ice-water and stirred for 30 min. The mixture was basified with aq. NH₃ solution and extracted with DCM. The organic phase was washed with brine, dried (MgSO₄) and concentrated in vacuo. Purification by automated flash chromatography (CombiFlash Rf, Silica 24 g RediSep™ cartridge) eluting with a gradient of 0-47% EtOAc (3% TEA) in heptane afforded Example 652C as an orange oil (687 mg, 3.51 mmol, 62%). LRMS calculated for C₁H₁₄ClN: 195; found: 196 (M+H). ¹H NMR (400 MHz, DMSO-d₆) δ ppm: 8.30-8.27 (m, 1H), 7.36-7.32 (m, 1H), 2.91-2.84 (m, 2H), 2.54 (s, 2H), 1.63 (t, J=6.8 Hz, 2H), 1.00 (s, 6H).

### Example 652 (1r,2′S,4S)-4-(3-chloroanilino)-2′-{(2R)-3-[(6,6-dimethyl-5,6,7,8-tetrahydroquinolin-4-yl)oxy]-2-methylpropyl}-2′,3′-dihydrospiro[cyclohexane-1,1′-indene]-4-carboxylic acid

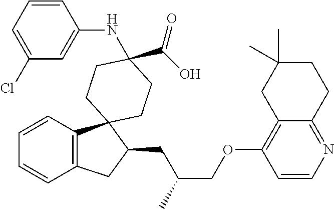 [SMILES: C[C@@H](COc1ccnc2c1CC(C)(C)CC2)C[C@H]1Cc2ccccc2[C@]12CC[C@@](Nc1cccc(Cl)c1)(C(=O)O)CC2]

Using General procedure 31c and Preparation 10b1 (150 mg, 0.34 mmol, 1 eq) as the appropriate alcohol and Example 652C (146 mg, 0.75 mmol, 2.2 eq) as the appropriate aryl chloride, Example 652 was obtained as an off-white powder (111 mg, 0.19 mmol, 56%). LRMS calculated for C₃₆H₄₃ClN₂O₃: 586; found: 587 (M+H). ¹H NMR (400 MHz, DMSO-d₆) δ ppm: 8.17 (d, J=5.6 Hz, 1H), 7.39-7.32 (m, 1H), 7.23-7.11 (m, 3H), 7.05 (t, J=8.1 Hz, 1H), 6.78 (d, J=5.6 Hz, 1H), 6.63 (t, J=2.1 Hz, 1H), 6.58-6.52 (m, 2H), 3.87 (d, J=6.3 Hz, 2H), 3.00 (dd, J=15.6, 7.0 Hz, 1H), 2.79-2.71 (m, 2H), 2.59-2.38 (m, 2H), 2.37-2.25 (m, 2H), 2.22-2.11 (m, 2H), 2.06-1.79 (m, 4H), 1.79-1.68 (m, 1H), 1.58-1.36 (m, 5H), 1.35-1.25 (m, 1H), 1.04 (d, J=6.6 Hz, 3H), 0.92 (s, 3H), 0.89 (s, 3H).

### Example 653 (1r,2′S,4S)-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

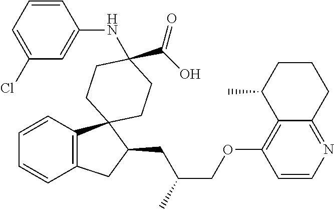 [SMILES: C[C@@H](COc1ccnc2c1[C@H](C)CCC2)C[C@H]1Cc2ccccc2[C@]12CC[C@@](Nc1cccc(Cl)c1)(C(=O)O)CC2]

Using General procedure 32 and Preparation 10b1 as the appropriate indane and Preparation 2a1 as the appropriate alcohol Example 653 was obtained. HRMS calculated for C₃₅H₄₁N₂O₃Cl: 572.2806; found: 573.2870 (M+H).

### Example 654 (1r,2′S,4S)-4-(3-chloroanilino)-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylic acid

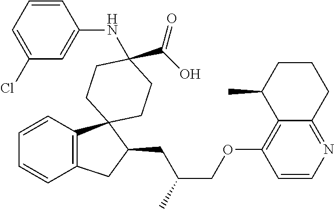 [SMILES: C[C@@H](COc1ccnc2c1[C@@H](C)CCC2)C[C@H]1Cc2ccccc2[C@]12CC[C@@](Nc1cccc(Cl)c1)(C(=O)O)CC2]

Using General procedure 32 and Preparation 10b1 as the appropriate indane and Preparation 2a2 as the appropriate alcohol Example 654 was obtained. HRMS calculated for C₃₅H₄₁N₂O₃Cl: 572.2806; found: 573.2871 (M+H).

### Example 655

### Example 655A 5-methyl-1-oxo-5,6,7,8-tetrahydro-1λ⁵-quinoline

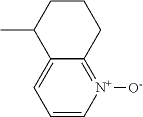

5-methyl-5,6,7,8-tetrahydroquinoline (2.00 g, 13.6 mmol) was dissolved in DCM (7 mL) and cooled to 0° C. MeReO₃ (169 mg, 0.68 mmol) and H₂O₂ (30%, 2 mL) were added and the mixture was stirred until no further conversion was observed. MnO₂ was added and the mixture was concentrated under reduced pressure. Toluene was added and the mixture was concentrated under reduced pressure. The crude product was purified via flash chromatography using DCM and MeOH as eluents to obtain Example 655A as a racemate. ¹H NMR (500 MHz, DMSO-d₆) δ ppm: 8.11 (m, 1H), 7.21 (m, 1H), 7.20 (m, 1H), 2.90 (m, 1H), 2.77-2.62 (m, 2H), 1.84+1.73 (m+m, 2H), 1.77+1.45 (m+m, 2H), 1.22 (d, 3H).

### Example 655B 5-methyl-4-nitro-1-oxo-5,6,7,8-tetrahydro-1λ⁵-quinoline

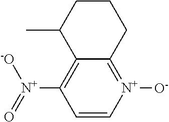

cc. H₂SO₄ (2 mL, 37 mmol) and cc. HNO₃ (1 mL, 24 mmol) were mixed in a dry flask at 0° C. under N₂ atmosphere. This mixture was added dropwise to Example 655A (0.51 g, 3.12 mmol) at 0° C. under N₂ atmosphere. The mixture was stirred at rt overnight then at 45° C. for a week. The mixture was allowed to cool to rt and it was poured onto ice and the pH was set to 10 with 2 M aq. NaOH solution. Brine was added and the mixture was extracted with DCM. The combined organic layers were dried over Na₂SO₄, filtered and the filtrate was concentrated under reduced pressure. The crude product was purified via prep RP-HPLC using 25 mM aq. NH₄HCO₃ solution and MeCN as eluents to obtain Example 655B. ¹H NMR (500 MHz, DMSO-d₆) δ ppm: 8.36 (d, 1H), 7.92 (d, 1H), 3.65 (m, 1H), 2.92+2.59 (m+m, 2H), 1.86 (m, 2H), 1.69 (m, 2H), 1.18 (d, 3H).

### Example 655C 5-methyl-5,6,7,8-tetrahydroquinolin-4-amine

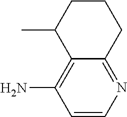

Example 655B (110 mg, 0.53 mmol) was dissolved in MeOH (15 mL), then 10% Pd/C (11 mg) was added to the mixture. The autoclave was evacuated and backfilled with N₂ (×3), then evacuated and filled with H₂. The reaction mixture was stirred at rt at 4.5 bar overnight. Then 10% Pd/C (10 mg) was added to the mixture. The autoclave was evacuated and backfilled with N₂ (×3), then evacuated and filled with H₂. The reaction mixture was stirred at rt at 4.5 bar until no further conversion was observed. The reaction mixture was filtered, washed with MeOH and THF. The filtrate was concentrated under reduced pressure to obtain Example 655C. LRMS calculated for C₁₀H₁₄N₂: 162.1; found: 179.2 (M+NH₄).

### Example 655D tert-butyl N-(5-methyl-5,6,7,8-tetrahydroquinolin-4-yl)carbamate

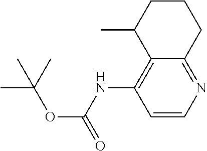 [SMILES: CC1CCCc2nccc(NC(=O)OC(C)(C)C)c21]

Example 655C (100 mg, 0.57 mmol) was dissolved in THF (2 mL) and Boc₂0 (138 mg, 0.63 mmol) was added to the solution. The reaction mixture was stirred at rt until no further conversion was observed. The solvent was removed under reduced pressure and brine was added. The mixture was extracted with DCM and the combined organic layers were dried over Na₂SO₄, filtered and the filtrate was concentrated under reduced pressure. The crude product was purified via flash chromatography using DCM and MeOH as eluents to obtain Example 655D as a racemate. LRMS calculated for C₁₅H₂₂N₂O₂: 262.2; found: 207.2 (M-tBu+H).

### Example 655 (1r,2′S,4S)-4-(3-chloroanilino)-2′-{(2R)-2-methyl-3-[(5-methyl-5,6,7,8-tetrahydroquinolin-4-yl)amino]propyl}-2′,3′-dihydrospiro[cyclohexane-1,1′-indene]-4-carboxylic acid

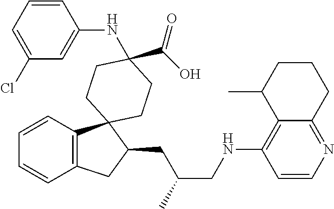 [SMILES: CC1CCCc2nccc(NC[C@H](C)C[C@H]3Cc4ccccc4[C@]34CC[C@@](Nc3cccc(Cl)c3)(C(=O)O)CC4)c21]

Using General procedure 32 and Preparation 10b1 as the appropriate indane and Example 655D as the appropriate amine, a mixture of diastereoisomers was obtained. It was dissolved in DCM, then TFA (100 eq) was added. The reaction mixture was stirred at rt overnight. The mixture was slowly quenched with TEA, then it was concentrated under reduced pressure. The crude product was purified via prep RP-HPLC using 25 mM aq. NH₄HCO₃ solution and MeCN as eluents to obtain Example 655 as a mixture of diastereoisomers. HRMS calculated for C₃₅H₄₂N₃O₂Cl: 571.2966; found: 572.3024 (M+H).

### Example 656 and Example 657

### Example 656A 5-ethylidene-5,6,7,8-tetrahydroquinoline

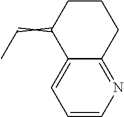

To a suspension of ethyl triphenylphosphonium bromide (10.59 g, 28.54 mmol, 1.4 eq) in THE (60 mL), cooled to 0° C. under N₂, was added nBuLi (12.41 mL, 2.3 M, 28.54 mmol, 1.4 eq) dropwise. The mixture was stirred at 0° C. for 30 min, followed by the addition of a solution of 7,8-dihydro-5 (6H)-quinolinone (2.5 mL, 20.38 mmol, 1 eq) in THE (15 mL). The suspension was stirred at rt for 12 h and then the mixture was cooled to 0° C. and quenched by the careful addition of sat. aq. NaHCO₃ solution (50 mL). Excess THE was removed in vacuo, and the residual mixture was extracted with EtOAc. The organic phase was dried (MgSO₄) and concentrated in vacuo. Purification by automated flash chromatography (Combiflash Rf, Silica 80 g RediSep™ cartridge) eluting with a gradient of 0-30% EtOAc in heptane afforded a 2:5 mixture of E/Z isomers, Example 656A, isolated as a colourless oil (957 mg, 6.01 mmol, 29%). LRMS calculated for C₁₁H₁₃N: 159; found: 160 (M+H). ¹H NMR (400 MHz, DMSO-d₆) δ ppm: 8.35/8.29 (dd, J=4.7, 1.7 Hz, 1H), 7.96/7.74 (dd, J=8.0, 1.7 Hz, 1H), 7.23-7.13 (m, 1H), 6.25-6.16/5.72-5.64 (m, 1H), 2.93-2.88/2.87-2.82 (m, 2H), 2.49-2.43/2.40-2.35 (m, 2H), 1.93-1.76 (m, 5H).

### Example 656B 5-ethyl-5,6,7,8-tetrahydroquinoline

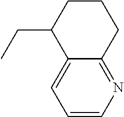

To a solution of Example 656A (958 mg, 6.02 mmol, 1 eq) in EtOH (40 mL) was added catalytic Pd/C (50 mg). The mixture was evacuated and backfilled with N₂ (×3), then evacuated and backfilled with H₂ and shaken at rt under an atmosphere of H₂ for 3 h. The mixture was filtered through celite, washed with EtOH and the solvent removed in vacuo to afford a racemate, Example 656B, isolated as a yellow oil. LRMS calculated for C₁₁H₁₅N: 161; found: 162 (M+H). ¹H NMR (400 MHz, DMSO-d₆) δ ppm: 8.33-8.29 (m, 1H), 7.67-7.62 (m, 1H), 7.21-7.16 (m, 1H), 2.89-2.67 (m, 3H), 1.95-1.59 (m, 5H), 1.59-1.46 (m, 1H), 0.92 (t, J=7.4 Hz, 3H).

### Example 656C 5-ethyl-1-oxo-5,6,7,8-tetrahydro-1)⁵-quinoline

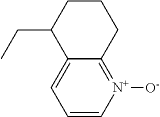

mCPBA (70% purity, 1.98 g, 8.04 mmol, 1.2 eq) was added to a solution of Example 656B (1.08 g, 6.7 mmol, 1 eq) in DCM (30 mL), cooled to 0° C. and the mixture was stirred for 2 h. 2 M aq. NaOH solution was added, and the mixture was stirred for 20 min. The layers were separated, and the organic phase was dried (MgSO₄) and concentrated in vacuo to afford a racemate, Example 656C, isolated as a yellow solid (1.02 g, 5.75 mmol, 86%). LRMS calculated for C_{II}H₁₅NO: 177; found: 178 (M+H). ¹H NMR (400 MHz, DMSO-d₆) δ ppm: 8.16-8.09 (m, 1H), 7.25-7.18 (m, 2H), 2.81-2.59 (m, 3H), 1.89-1.46 (m, 6H), 0.92 (t, J=7.4 Hz, 3H).

### Example 656D 4-chloro-5-ethyl-5,6,7,8-tetrahydroquinoline

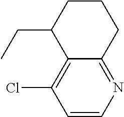

Example 656C (1 g, 5.64 mmol, 1 eq) was added portionwise to POCl₃ (5.26 mL, 56.42 mmol, 10 eq), cooled to 0° C., over 15 min. The mixture was stirred at rt until all the material had dissolved, and then heated at 100° C. for 3 h. A mixture of regioisomers was obtained. The mixture was allowed to cool to rt, and then quenched by the dropwise addition to rapidly stirred ice-water and then stirred for 20 min. The mixture was cooled to 0° C. and basified by the slow addition of aq. NH₃ solution. The mixture was extracted with DCM, and the combined organic extracts dried (MgSO₄) and concentrated in vacuo. Purification by automated flash chromatography (Combiflash Rf, Silica 24 g RediSep™ cartridge) eluting with a gradient of 0-22% EtOAc in DCM afforded a racemate, Example 656D, isolated as a yellow oil (401 mg, 2.05 mmol, 36%). LRMS calculated for C₁₁H₁₄ClN: 195; found: 196 (M+H). ¹H NMR (400 MHz, DMSO-d₆) δ ppm: 8.27 (d, J=5.2 Hz, 1H), 7.33 (d, J=5.2 Hz, 1H), 2.95-2.84 (m, 2H), 2.84-2.71 (m, 1H), 1.99-1.73 (m, 3H), 1.67-1.33 (m, 3H), 0.99 (t, J=7.4 Hz, 3H).

### Example 656E 5-ethyl-5,6,7,8-tetrahydroquinolin-4-ol

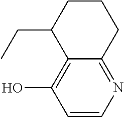

Example 656D (210 mg, 1.07 mmol, 1 eq) and K₂CO₃ (445 mg, 3.22 mmol, 3 eq) were combined in a mixture of DMF (2 mL) and water (0.2 mL) and the mixture was sparged with N₂. tBuXPhos (36 mg, 0.09 mmol, 0.08 eq) and Herrmann's catalyst (20 mg, 21.46 μmol, 0.02 eq) were added, and the mixture was sparged with N₂ and then heated at 115° C. for 45 min under microwave irradiation. The mixture was allowed to cool to rt, diluted with DCM and washed with 1 M aq. HCl solution. The organic phase was discarded, and the aq. phase was adjusted to pH8 with sat. aq. NaHCO₃ solution and extracted with DCM/IPA (3:1). The combined organic extracts were dried (MgSO₄) and concentrated in vacuo to afford a racemate, Example 656E, isolated as a cream wax (142 mg, 0.8 mmol, 75%). LRMS calculated for C₁₁H₁₅NO: 177; found: 178 (M+H). ¹H NMR (400 MHz, DMSO-d₆) δ ppm: 11.12 (br s, 1H), 7.47 (d, J=7.2 Hz, 1H), 5.96 (d, J=7.2 Hz, 1H), 2.62-2.46 (m, 3H), 1.83-1.61 (m, 4H), 1.49-1.36 (m, 1H), 1.18-1.03 (m, 1H), 0.89 (t, J=7.3 Hz, 3H).

### Example 656F methyl (1r,2′S,4S)-4-(3-chloroanilino)-2′-[(2R)-3-{[(5S)-5-ethyl-5,6,7,8-tetrahydroquinolin-4-yl]oxy}-2-methylpropyl]-2′,3′-dihydrospiro[cyclohexane-1,1′-indene]-4-carboxylate

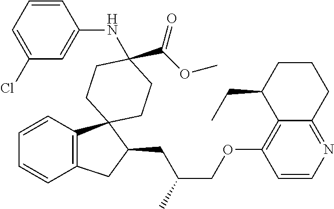 [SMILES: CC[C@H]1CCCc2nccc(OC[C@H](C)C[C@H]3Cc4ccccc4[C@]34CC[C@@](Nc3cccc(Cl)c3)(C(=O)OC)CC4)c21]

### Example 657A methyl (1r,2′S,4S)-4-(3-chloroanilino)-2′-[(2R)-3-{[(5R)-5-ethyl-5,6,7,8-tetrahydroquinolin-4-yl]oxy}-2-methylpropyl]-2′,3′-dihydrospiro[cyclohexane-1,1′-indene]-4-carboxylate

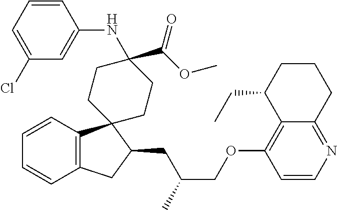 [SMILES: CC[C@@H]1CCCc2nccc(OC[C@H](C)C[C@H]3Cc4ccccc4[C@]34CC[C@@](Nc3cccc(Cl)c3)(C(=O)OC)CC4)c21]

Using General procedure 30a and Preparation 10b1 as the appropriate indane and Example 656E as the appropriate alcohol a mixture of diastereoisomers was obtained. The diastereoisomers were separated by chiral chromatography. Column: AD, 50×500 mm, 20 μm, Eluents: EtOH/heptane. The diastereoisomer eluting earlier was collected as Example 656F. ¹H NMR (500 MHz, DMSO-d₆) δ ppm: 8.14 (d, 1H), 7.41-7.11 (m, 4H), 7.05 (t, 1H), 6.77 (d, 1H), 6.60 (t, 1H), 6.56 (dm, 1H), 6.43 (dm, 1H), 6.30 (s, 1H), 3.93/3.83 (dd+dd, 2H), 3.64 (s, 3H), 3.00/2.54 (dd+dd, 2H), 2.74/2.64 (m+m, 2H), 2.71 (m, 1H), 2.50-2.18 (m, 12H), 2.14 (m, 1H), 2.14 (m, 1H), 1.59/1.28 (m+m, 2H), 1.46/1.36 (m+m, 2H), 1.05 (d, 3H), 0.83 (t, 3H). HRMS calculated for C₃₇H₄₅N₂O₃Cl: 600.3119; found: 601.3186 (M+H).

The diastereoisomer eluting later was collected as Example 657A. ¹H NMR (500 MHz, DMSO-d₆) δ ppm: 8.15 (d, 1H), 7.43-7.11 (m, 4H), 7.04 (t, 1H), 6.79 (d, 1H), 6.58 (t, 1H), 6.55 (dm, 1H), 6.43 (dm, 1H), 6.31 (s, 1H), 3.88 (d, 2H), 3.64 (s, 3H), 3.00/2.54 (dd+dd, 2H), 2.75/2.66 (m+m, 2H), 2.73 (m, 1H), 2.50-1.14 (m, 12H), 2.13 (m, 1H), 2.00 (m, 1H), 1.54/1.22 (m+m, 2H), 1.53/1.35 (m+m, 2H), 1.03 (d, 3H), 0.74 (t, 3H). HRMS calculated for C₃₇H₄₅N₂O₃Cl: 600.3119; found: 601.3190 (M+H).

### Example 656 (1r,2′S,4S)-4-(3-chloroanilino)-2′-[(2R)-3-{[(5S)-5-ethyl-5,6,7,8-tetrahydroquinolin-4-yl]oxy}-2-methylpropyl]-2′,3′-dihydrospiro[cyclohexane-1,1′-indene]-4-carboxylic acid

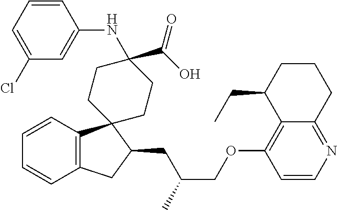 [SMILES: CC[C@H]1CCCc2nccc(OC[C@H](C)C[C@H]3Cc4ccccc4[C@]34CC[C@@](Nc3cccc(Cl)c3)(C(=O)O)CC4)c21]

Using General procedure 33a and Example 656F as the appropriate ester Example 656 was obtained. ¹H NMR (500 MHz, DMSO-d₆) δ ppm: 12.69 (br s, 1H), 8.14 (d, 1H), 7.42-7.10 (m, 4H), 7.04 (t, 1H), 6.77 (d, 1H), 6.62 (t, 1H), 6.54 (dm, 1H), 6.52 (dm, 1H), 6.22 (br s, 1H), 3.93/3.83 (dd+dd, 2H), 3.00/2.54 (dd+dd, 2H), 2.75/2.64 (m+m, 2H), 2.72 (m, 1H), 2.50-1.33 (m, 12H), 2.14 (m, 1H), 2.00 (m, 1H), 1.59/1.28 (m+m, 2H), 1.46/1.36 (m+m, 2H), 1.05 (d, 3H), 0.83 (t, 3H). HRMS calculated for C₃₆H₄₃N₂O₃Cl: 586.2962; found: 587.3024 (M+H).

### Example 657 (1r,2′S,4S)-4-(3-chloroanilino)-2′-[(2R)-3-{[(5R)-5-ethyl-5,6,7,8-tetrahydroquinolin-4-yl]oxy}-2-methylpropyl]-2′,3′-dihydrospiro[cyclohexane-1,1′-indene]-4-carboxylic acid

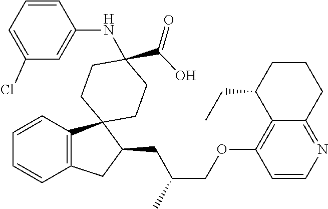 [SMILES: CC[C@@H]1CCCc2nccc(OC[C@H](C)C[C@H]3Cc4ccccc4[C@]34CC[C@@](Nc3cccc(Cl)c3)(C(=O)O)CC4)c21]

Using General procedure 33a and Example 657A as the appropriate ester Example 657 was obtained. ¹H NMR (500 MHz, DMSO-d₆) δ ppm: 12.71 (br s, 1H), 8.14 (d, 1H), 7.43-7.10 (m, 4H), 7.03 (t, 1H), 6.78 (d, 1H), 6.61 (t, 1H), 6.53 (dm, 2H), 6.23 (br s, 1H), 3.89/3.87 (dd+dd, 2H), 3.00/2.54 (dd+dd, 2H), 2.75/2.65 (m+m, 2H), 2.74 (m, 1H), 2.50-1.28 (m, 12H), 2.14 (m, 1H), 2.00 (m, 1H), 1.55/1.22 (m+m, 2H), 1.53/1.36 (m+m, 2H), 1.03 (d, 3H), 0.75 (t, 3H). HRMS calculated for C₃₆H₄₃N₂O₃Cl: 586.2962; found: 587.3039 (M+H).

### Example 658 and Example 659 and Example 660 and Example 661

### Example 658A methyl 4-hydroxy-5,8-dimethylquinoline-2-carboxylate

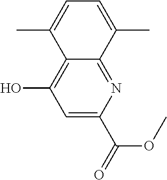

2,5-dimethylaniline (12 g, 99 mmol) was dissolved in MeOH (100 mL), then dimethyl but-2-ynedioate (15.48 g, 109 mmol) was added dropwise while temperature was maintained between 20 and 25° C., and then the mixture was stirred under N₂ atmosphere at 24° C. for 20 min to reach complete conversion. The reaction mixture was concentrated to dryness under reduced pressure, then heptane (100 mL) was added, and the mixture was concentrated again under reduced pressure. The obtained adduct (27.8 g, 105.6 mmol) was added slowly (5 min) to the stirred Eaton's reagent (79 mL, 1.5 g/mL P₂O₅ in methane sulfonic acid) at 65° C., then the mixture was stirred at 65° C. until no further conversion was observed. K₂CO₃ (79 g) was dissolved in water (300 mL), then ice (600 mL) was added. The crude reaction mixture was added dropwise from a dropping funnel into the K₂CO₃ solution. The pH was set to 6-7 with solid K₂CO₃, then the mixture was stirred for 15 min, then cooled to 0° C. The cooled mixture was filtered, precipitates were washed with water (2×200 mL) and dried to obtain Example 658A. ¹H NMR (500 MHz, DMSO-d₆) δ ppm: 7.42 (d, 1H), 7.08 (d, 1H), 6.95 (br s, 1H), 3.94 (s, 3H), 2.76 (s, 3H), 2.54 (s, 3H). HRMS calculated for C₁₃H₁₃NO₃: 231.0895; found: 232.0970 (M+H).

### Example 658B methyl 4-hydroxy-5,8-dimethyl-5,6,7,8-tetrahydroquinoline-2-carboxylate

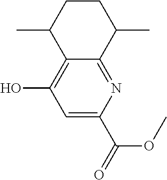

Example 658A (23.2 g, 100 mmol) was dissolved in TFA (250 mL). PtO₂ (1.16 g, 5.1 mmol) was added and the autoclave was evacuated and backfilled with N₂ (×3), then evacuated and filled with H₂. The reaction mixture was stirred at 4 bar at 80° C. for 5 h. The reaction mixture was filtered, washed with MeOH, and the filtrate was concentrated under reduced pressure. MeOH/NH₃ (6 M, 50 mL) was added and the mixture was concentrated under reduced pressure The crude product was purified via flash chromatography using EtOAc and 1.2% NH₃ in MeOH as eluents to obtain Example 658B as a racemic mixture of diastereoisomers. ¹H NMR (500 MHz, DMSO-d₆) δ ppm: 7.23 (s, 1H), 3.81 (s, 3H), 3.06 (m, 1H), 2.74 (m, 1H), 1.81/1.55 (m+m, 2H), 1.70/1.57 (m+m, 2H), 1.29 (d, 3H), 1.14 (d, 3H).

### Example 658C 4-hydroxy-5,8-dimethyl-5,6,7,8-tetrahydroquinoline-2-carboxylic acid

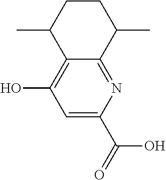 [SMILES: CC1CCC(C)c2c(O)cc(C(=O)O)nc21]

A solution of Example 658B (16.2 g, 81.2 mmol) and NaOH (16.2 g, 406 mmol) in THE (245 mL) and in water (80 mL) was stirred at 50° C. for 45 min. The pH was set to 1 with cc. aq. HCl solution (29 mL) and 2 M aq. HCl (35 mL) solution. THE was removed under reduced pressure and the mixture was freeze-dried to obtain Example 658C which contained NaCl (about 5 eq.). LRMS calculated for C₁₂H₁₅NO₃: 221.1; found: 222.2 (M+H).

### Example 658D 5,8-dimethyl-5,6,7,8-tetrahydroquinolin-4-ol

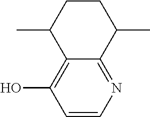

The solution of Example 658C (40.2 g, contains 5 eq. NaCl) in Ph₂O (120 mL) was stirred at 260° C. for 50 min. The mixture was allowed to cool to rt. Heptane (550 mL) was added and the mixture was sonicated for 3 min. It was stirred at rt for 15 min, then filtered, the precipitate was washed with heptane, then it was dissolved in MeOH, silica gel was added and it was concentrated under reduced pressure. Then it was purified via flash chromatography using EtOAc and 1.2% NH₃ in MeOH as eluents. Then it was refluxed with DIPE (200 mL), then cooled to rt, filtered, washed with DIPE. The precipitate was dried under reduced pressure to give Example 658D. ¹H NMR (500 MHz, DMSO-d₆) δ ppm: 11.07/10.91 (br s, 1H), 7.49 (d, 1H), 5.98/5.94 (d, 1H), 2.87/2.85 (m, 1H), 2.71/2.68 (m, 1H), 2.05-1.34 (m, 4H), 1.24/1.19 (d, 3H), 1.07/1.03 (d, 3H).

### Example 658E (5R*,8S*)-5,8-dimethyl-5,6,7,8-tetrahydroquinolin-4-ol, enantiomer 1

and

### Example 658F (5R*,8S*)-5,8-dimethyl-5,6,7,8-tetrahydroquinolin-4-ol, enantiomer 2

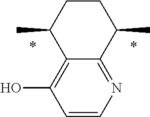

and

### Example 658G (5R,8R)-5,8-dimethyl-5,6,7,8-tetrahydroquinolin-4-ol

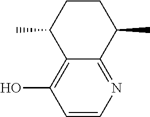

### Example 658H (5S,8S)-5,8-dimethyl-5,6,7,8-tetrahydroquinolin-4-ol

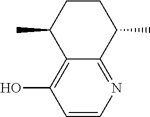

To the solution of Example 658D (2.00 g, 11.28 mmol) and TEA (2 mL, 14.67 mmol) in CHCl₃ (45 mL) AcCl (1.04 mL, 14.67 mmol) was added dropwise, while the temperature was maintained between 10 and 25° C. using ice cooled bath. Then the mixture was stirred at 25° C. for 20 min. Then sat. aq. NaHCO₃ solution (10 mL), then water (10 mL) were added, then phases were separated. The aq. phase was extracted with CHCl₃ (10 mL). The combined organic layers were dried over MgSO₄, filtered, and the filtrate was concentrated onto silica. The cis and trans isomers were separated by flash chromatography using heptane and EtOAc than EtOAc and 1.2% NH₃ in MeOH as eluents. The separated isomers were dissolved in MeOH (5-5 mL), then NH₃/MeOH (6 M, 2-2 mL) was added and they were stirred at rt for 15 min. The reaction mixtures were filtered and concentrated under reduced pressure. The enantiomers of cis isomer were separated by chiral chromatography. Column: AS-V, 100×500 mm, 20 μm, Eluents: EtOH/heptane+0.05% DEA. The enantiomer eluting earlier was collected as Example 658E. ¹H NMR (500 MHz, DMSO-d₆) δ ppm: 10.81 (br s, 1H), 7.44 (t, 1H), 5.92 (d, 1H), 2.85 (m, 1H), 2.67 (m, 1H), 1.79/1.50 (m+m, 2H), 1.61/1.50 (m+m, 2H), 1.24 (d, 3H), 1.06 (d, 3H).

The enantiomer eluting later was collected as Example 658F.

The enantiomers of trans isomer were separated by chiral chromatography. Column: AD, 50×500 mm, 20 μm, Eluents: EtOH/heptane+0.05% DEA. The enantiomer eluting earlier was collected as Example 658G. ¹H NMR (500 MHz, DMSO-d₆) δ ppm: 11.01 (br s, 1H), 7.47 (d, 1H), 5.91 (d, 1H), 2.84 (m, 1H), 2.69 (m, 1H), 1.95/1.44 (td+d, 2H), 1.77/1.39 (td+d, 2H), 1.18 (d, 3H), 1.02 (d, 3H).

The enantiomer eluting later was collected as Example 658H.

### Example 658 (1r,2′S,4S)-4-(3-chloroanilino)-2′-[(2R)-3-{[(5R*,8S*)-5,8-dimethyl-5,6,7,8-tetrahydroquinolin-4-yl]oxy}-2-methylpropyl]-2′,3′-dihydrospiro[cyclohexane-1,1′-indene]-4-carboxylic acid, diastereoisomer 1

### Example 659 (1r,2′S,4S)-4-(3-chloroanilino)-2′-[(2R)-3-{[(5R*,8S*)-5,8-dimethyl-5,6,7,8-tetrahydroquinolin-4-yl]oxy}-2-methylpropyl]-2′,3′-dihydrospiro[cyclohexane-1,1′-indene]-4-carboxylic acid, diastereoisomer 2

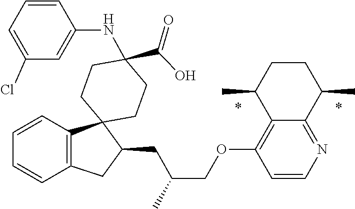 [SMILES: C[C@@H](COc1ccnc2c1[C@@H](C)CC[C@H]2C)C[C@H]1Cc2ccccc2[C@]12CC[C@@](Nc1cccc(Cl)c1)(C(=O)O)CC2]

Using General procedure 32 and Preparation 10b1 as the appropriate indane and Example 658E as the appropriate alcohol Example 658 was obtained. ¹H NMR (500 MHz, DMSO-d₆) δ ppm: 12.71 (br s, 1H), 8.20 (d, 1H), 7.36 (d, 1H), 7.20 (d, 1H), 7.15 (tm, 2H), 7.04 (t, 1H), 6.76 (d, 1H), 6.63 (t, 1H), 6.53 (dm, 1H), 6.52 (dm, 1H), 6.22 (br s, 1H), 3.90/3.84 (dd+dd, 2H), 3.03 (m, 1H), 3.01/2.55 (dd+dd, 2H), 2.70 (m, 1H), 2.46-1.41 (m, 8H), 2.01 (m, 1H), 1.99 (m, 1H), 1.85/1.58 (m+m, 2H), 1.68/1.59 (m+m, 2H), 1.43/1.38 (m+m, 2H), 1.31 (d, 3H), 1.15 (d, 3H), 1.05 (d, 3H). HRMS calculated for C₃₆H₄₃N₂O₃Cl: 586.2962; found: 587.3023 (M+H).

Using General procedure 32 and Preparation 10b1 as the appropriate indane and Example 658F as the appropriate alcohol Example 659 was obtained. ¹H NMR (500 MHz, DMSO-d₆) δ ppm: 12.69 (br s, 1H), 8.20 (d, 1H), 7.37 (d, 1H), 7.20 (d, 1H), 7.15 (tm, 2H), 7.04 (t, 1H), 6.77 (d, 1H), 6.61 (t, 1H), 6.53 (dd, 1H), 6.53 (dd, 1H), 6.25 (br s, 1H), 3.89/3.86 (dd+dd, 2H), 3.07 (m, 1H), 3.00/2.54 (dd+dd, 2H), 2.71 (m, 1H), 2.71 (m, 1H), 2.44-1.36 (m, 8H), 2.13 (m, 1H), 1.84/1.55 (m+m, 2H), 1.72/1.58 (m+m, 2H), 1.49/1.35 (m+m, 2H), 1.31 (d, 3H), 1.06 (d, 3H), 1.05 (d, 3H). HRMS calculated for C₃₆H₄₃N₂O₃Cl: 586.2962; found: 587.3025 (M+H).

### Example 660 (1r,2′S,4S)-4-(3-chloroanilino)-2′-[(2R)-3-{[(5R,8R)-5,8-dimethyl-5,6,7,8-tetrahydroquinolin-4-yl]oxy}-2-methylpropyl]-2′,3′-dihydrospiro[cyclohexane-1,1′-indene]-4-carboxylic acid

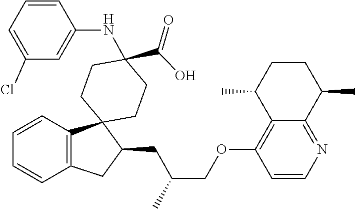 [SMILES: C[C@@H](COc1ccnc2c1[C@H](C)CC[C@H]2C)C[C@H]1Cc2ccccc2[C@]12CC[C@@](Nc1cccc(Cl)c1)(C(=O)O)CC2]

Using General procedure 32 and Preparation 10b1 as the appropriate indane and Example 658G as the appropriate alcohol Example 660 was obtained. ¹H NMR (500 MHz, DMSO-d₆) δ ppm: 12.71 (br s, 1H), 8.18 (d, 1H), 7.37 (d, 1H), 7.20 (d, 1H), 7.16 (t, 1H), 7.14 (t, 1H), 7.03 (t, 1H), 6.75 (d, 1H), 6.61 (dd, 1H), 6.53 (m, 2H), 6.24 (br s, 1H), 3.90/3.84 (dd+dd, 2H), 3.02 (m, 1H), 3.00/2.53 (dd+dd, 2H), 2.86 (m, 1H), 2.48-1.31 (m, 8H), 2.13 (dd, 1H), 2.04 (m, 1H), 2.00/1.50 (m+m, 2H), 1.86/1.44 (m+m, 2H), 1.50/1.37 (d+dd, 2H), 1.18 (d, 3H), 1.05 (d, 3H), 1.03 (d, 3H). HRMS calculated for C₃₆H₄₃N₂O₃Cl: 586.2962; found: 587.3026 (M+H).

### Example 661 (1r,2′S,4S)-4-(3-chloroanilino)-2′-[(2R)-3-{[(5S,8S)-5,8-dimethyl-5,6,7,8-tetrahydroquinolin-4-yl]oxy}-2-methylpropyl]-2′,3′-dihydrospiro[cyclohexane-1,1′-indene]-4-carboxylic acid

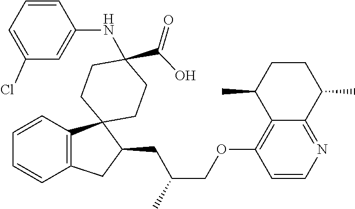 [SMILES: C[C@@H](COc1ccnc2c1[C@@H](C)CC[C@@H]2C)C[C@H]1Cc2ccccc2[C@]12CC[C@@](Nc1cccc(Cl)c1)(C(=O)O)CC2]

Using General procedure 32 and Preparation 10b1 as the appropriate indane and Example 658H as the appropriate alcohol Example 661 was obtained. ¹H NMR (500 MHz, DMSO-d₆) δ ppm: 12.71 (br s, 1H), 8.18 (d, 1H), 7.36 (d, 1H), 7.20 (d, 1H), 7.16 (t, 1H), 7.14 (t, 1H), 7.04 (t, 1H), 6.74 (d, 1H), 6.63 (dd, 1H), 6.54 (dd, 1H), 6.52 (dd, 1H), 6.22 (br s, 1H), 3.92/3.82 (dd+dd, 2H), 3.01/2.55 (dd+dd, 2H), 3.00 (m, 1H), 2.86 (m, 1H), 2.48-1.33 (m, 8H), 2.15 (m, 1H), 2.04/1.50 (m+m, 2H), 2.00 (m, 1H), 1.81/1.44 (m+m, 2H), 1.45/1.38 (m+m, 2H), 1.16 (d, 3H), 1.13 (d, 3H), 1.05 (d, 3H). HRMS calculated for C₃₆H₄₃N₂O₃Cl: 586.2962; found: 587.3021 (M+H).

### Example 662 and Example 663

### Example 662A 8-bromo-2,5-dimethyl-quinolin-4-ol

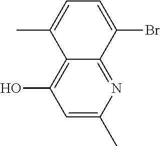

2-bromo-5-methyl-aniline (9.3 g, 50 mmol) was added to PPA (120 g) and the mixture was heated to 80° C. Ethyl 3-oxobutanoate (8.3 g, 64 mmol) was added and the mixture was stirred in 140° C. oil bath for 3 h. Then the reaction mixture was added to a cooled 20% aq. NaOH solution (600 mL). The precipitate was filtered and the solid was washed with water and dried to obtain Example 662A. ¹H NMR (500 MHz, DMSO-d₆) δ ppm: 9.92 (s, 1H), 7.76 (d, 1H), 6.93 (d, 1H), 5.91 (s, 1H), 2.74 (s, 3H), 2.38 (s, 3H).

### Example 662B 2,5-dimethyl-5,6,7,8-tetrahydroquinolin-4-ol, enantiomer 1

and

### Example 662C 2,5-dimethyl-5,6,7,8-tetrahydroquinolin-4-ol, enantiomer 2

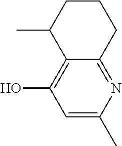

Example 662A (2.52 g, 10 mmol) was dissolved in AcOH (25 mL). Pd/C (10%, 0.25 g) was added to the mixture. The flask was evacuated and backfilled with N₂ (×3), then evacuated and filled with H₂. The reaction mixture was stirred at 70° C. overnight. Then TFA (100 mL) was added and the mixture was filtered through celite, the filtrate was concentrated under reduced pressure. The residue was dissolved in AcOH (100 mL), PtO₂ (0.18 g) was added and the flask was evacuated and backfilled with N₂ (×3), then evacuated and filled with H₂. The reaction mixture was stirred at 70° C. overnight. Then reaction mixture was filtered through celite, washed with TFA, and the filtrate was concentrated under reduced pressure. Then NH₃ in MeOH (6 M, 50 mL) was added and the mixture was concentrated under reduced pressure. The crude product was purified via flash chromatography using EtOAc and MeOH as eluents to obtain a racemate. The enantiomers were separated by chiral chromatography. Column: AS-V, 100×500 mm, 20 μm, Eluents: EtOH/heptane+0.05% DEA. The enantiomer eluting earlier was collected as Example 662B. The enantiomer eluting later was collected as Example 662C. ¹H NMR (500 MHz, DMSO-d₆) S ppm: 13.58 (br s, 1H), 6.81 (s, 1H), 3.03 (m, 1H), 2.86/2.77 (dm+m, 2H), 2.47 (s, 3H), 1.88-1.62 (m, 4H), 1.15 (d, 3H).

### Example 662 (1r,2′S,4S)-4-(3-chloroanilino)-2′-{(2R)-3-[(2,5-dimethyl-5,6,7,8-tetrahydroquinolin-4-yl)oxy]-2-methylpropyl}-2′,3′-dihydrospiro[cyclohexane-1,1′-indene]-4-carboxylic acid, diastereoisomer 1

### Example 663 (1r,2′S,4S)-4-(3-chloroanilino)-2′-{(2R)-3-[(2,5-dimethyl-5,6,7,8-tetrahydroquinolin-4-yl)oxy]-2-methylpropyl}-2′,3′-dihydrospiro[cyclohexane-1,1′-indene]-4-carboxylic acid, diastereoisomer 2

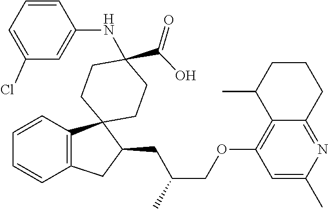 [SMILES: Cc1cc(OC[C@H](C)C[C@H]2Cc3ccccc3[C@]23CC[C@@](Nc2cccc(Cl)c2)(C(=O)O)CC3)c2c(n1)CCCC2C]

Using General procedure 32 and Preparation 10b1 as the appropriate indane and Example 662B as the appropriate alcohol Example 662 was obtained. ¹H NMR (500 MHz, DMSO-d₆) δ ppm: 12.72 (br s, 1H), 7.40-7.10 (m, 4H), 7.04 (t, 1H), 6.63 (s, 1H), 6.63 (t, 1H), 6.54 (dm, 1H), 6.52 (dm, 1H), 6.23 (m, 1H), 3.89/3.81 (dd+dd, 2H), 3.01/2.55 (dd+dd, 2H), 2.95 (m, 1H), 2.69/2.59 (dm+m, 2H), 2.49-1.34 (m, 8H), 2.30 (s, 3H), 2.15 (m, 1H), 1.98 (br, 1H), 1.78/1.71 (m, 2H), 1.59 (m, 2H), 1.43/1.36 (m+m, 2H), 1.12 (d, 3H), 1.05 (d, 3H). HRMS calculated for C₃₆H₄₃N₂O₃Cl: 586.2962; found: 587.3022 (M+H).

Using General procedure 32 and Preparation 10b1 as the appropriate indane and Example 662C as the appropriate alcohol Example 663 was obtained. ¹H NMR (500 MHz, DMSO-d₆) δ ppm: 12.72 (br s, 1H), 7.42-7.10 (m, 4H), 7.04 (t, 1H), 6.64 (s, 1H), 6.61 (t, 1H), 6.53 (dd, 1H), 6.53 (dd, 1H), 6.26 (m, 1H), 3.88/3.83 (dd+dd, 2H), 3.00/2.53 (dd+dd, 2H), 2.99 (m, 1H), 2.69/2.60 (dm+m, 2H), 2.49-1.32 (m, 8H), 2.31 (s, 3H), 2.14 (m, 1H), 1.99 (m, 1H), 1.78/1.67 (m, 2H), 1.66/1.58 (m+m, 2H), 1.49/1.33 (m+m, 2H), 1.04 (d, 3H), 1.01 (d, 3H). HRMS calculated for C₃₆H₄₃N₂O₃Cl: 586.2962; found: 587.3025 (M+H).

### Example 664 and Example 665 and Example 666 and Example 667

### Example 664A 5,7-dimethylquinolin-4-ol

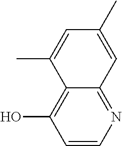

3,5-dimethylaniline (2.42 g, 20 mmol) and Meldrum's acid (4.1 g, 22 mmol) were suspended in dry EtOH (90 mL). The mixture was stirred at rt for 1 h. Then the mixture was concentrated under reduced pressure and the residue was suspended in DIPE (60 mL) and filtered. The solid was washed with DIPE (20 mL) and dried. Then it was added to PPA (50 g) and the mixture was heated at 130° C. for 4 h. Then the mixture was added to a cooled 20% aq. NaOH solution (200 mL). The precipitate was filtered and the solid was washed with water and dried to obtain Example 664A. ¹H NMR (500 MHz, DMSO-d₆) δ ppm: 11.39 (s, 1H), 7.69 (d, 1H), 7.09 (s, 1H), 6.81 (s, 1H), 5.89 (d, 1H), 2.75 (s, 3H), 2.32 (s, 3H).

### Example 664B (5S,7R)-5,7-dimethyl-5,6,7,8-tetrahydroquinolin-4-ol

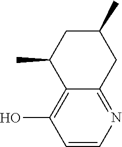

and

### Example 664C (5R,7S)-5,7-dimethyl-5,6,7,8-tetrahydroquinolin-4-ol

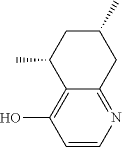

and

### Example 664D (5S,7S)-5,7-dimethyl-5,6,7,8-tetrahydroquinolin-4-ol

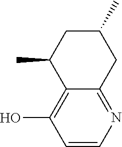

and

### Example 664E (5R,7R)-5,7-dimethyl-5,6,7,8-tetrahydroquinolin-4-ol

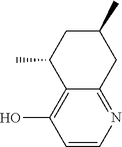

Example 664A (3.20 g, 18.47 mmol) was dissolved in AcOH (100 mL). PtO₂ (0.3 g) and TFA (4.62 mL) were added to the mixture. The flask was evacuated and backfilled with N₂ (×3), then evacuated and filled with H₂. The reaction mixture was stirred at 70° C. overnight. The reaction mixture was filtered through celite, washed with TFA, and the filtrate was concentrated under reduced pressure. The crude product was purified via flash chromatography using DCM and MeOH as eluents to obtain a racemic mixture of diastereoisomers. The diastereoisomers were separated via prep RP-HPLC using 25 mM aq. NH₄HCO₃ solution and MeCN as eluents. The enantiomers of the secondly eluting diastereoisomer were separated by chiral chromatography. Column: IC, 50×500 mm, 20 μm, Eluents: MeOH/EtOH/heptane+0.05% DEA. The enantiomer eluting earlier was collected as Example 664B. ¹H NMR (500 MHz, DMSO-d₆) S ppm: 11.00 (br s, 1H), 7.41 (t, 1H), 5.89 (d, 1H), 2.71 (m, 1H), 2.42/2.17 (dd+dd, 2H), 1.93/0.91 (m+dd, 2H), 1.63 (d, 1H), 1.18 (d, 3H), 0.98 (m, 3H).

The enantiomer eluting later was collected as Example 664C.

The enantiomers of the firstly eluted diastereoisomer were separated by chiral chromatography. Column: ID, 50×500 mm, 20 μm, Eluents: MeOH/EtOH/heptane+0.05% DEA. The enantiomer eluting earlier was collected as Example 664D. ¹H NMR (400 MHz, DMSO-d₆) δ ppm: 11.01 (br s, 1H), 7.44 (t, 1H), 5.90 (d, 1H), 2.87 (m, 1H), 2.53/2.10 (dd+dd, 2H), 1.94 (m, 1H), 1.51/1.31 (d+t, 2H), 1.05 (d, 3H), 1.00 (d, 3H).

The enantiomer eluting later was collected as Example 664E.

### Example 664 (1r,2′S,4S)-4-(3-chloroanilino)-2′-[(2R)-3-{[(5S,7R)-5,7-dimethyl-5,6,7,8-tetrahydroquinolin-4-yl]oxy}-2-methylpropyl]-2′,3′-dihydrospiro[cyclohexane-1,1′-indene]-4-carboxylic acid

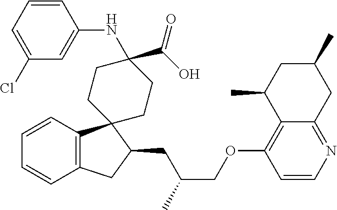 [SMILES: C[C@H]1Cc2nccc(OC[C@H](C)C[C@H]3Cc4ccccc4[C@]34CC[C@@](Nc3cccc(Cl)c3)(C(=O)O)CC4)c2[C@@H](C)C1]

Using General procedure 32 and Preparation 10b1 as the appropriate indane and Example 664B as the appropriate alcohol Example 664 was obtained. ¹H NMR (500 MHz, DMSO-d₆) δ ppm: 12.67 (br s, 1H), 8.12 (d, 1H), 7.40-7.11 (m, 4H), 7.04 (t, 1H), 6.78 (d, 1H), 6.62 (t, 1H), 6.54 (dm, 1H), 6.52 (dm, 1H), 6.23 (m, 1H), 3.95/3.80 (dd+dd, 2H), 3.00/2.55 (dd+dd, 2H), 2.92 (m, 1H), 2.66/2.35 (dm+dd, 2H), 2.49-1.34 (m, 8H), 2.13 (m, 1H), 2.00 (m, 1H), 2.00/0.98 (m+m, 2H), 1.58 (m, 1H), 1.45/1.37 (m+m, 2H), 1.21 (d, 3H), 1.04 (d, 3H), 1.00 (d, 3H). HRMS calculated for C₃₆H₄₃N₂O₃Cl: 586.2962; found: 587.3039 (M+H).

### Example 665 (1r,2′S,4S)-4-(3-chloroanilino)-2′-[(2R)-3-{[(5R,7S)-5,7-dimethyl-5,6,7,8-tetrahydroquinolin-4-yl]oxy}-2-methylpropyl]-2′,3′-dihydrospiro[cyclohexane-1,1′-indene]-4-carboxylic acid

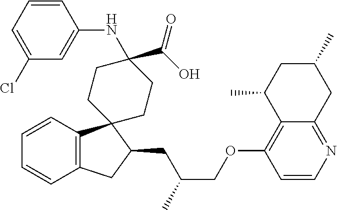 [SMILES: C[C@@H]1Cc2nccc(OC[C@H](C)C[C@H]3Cc4ccccc4[C@]34CC[C@@](Nc3cccc(Cl)c3)(C(=O)O)CC4)c2[C@H](C)C1]

Using General procedure 32 and Preparation 10b1 as the appropriate indane and Example 664C as the appropriate alcohol Example 665 was obtained. ¹H NMR (500 MHz, DMSO-d₆) δ ppm: 12.68 (br s, 1H), 8.12 (d, 1H), 7.41-7.10 (m, 4H), 7.04 (t, 1H), 6.79 (d, 1H), 6.61 (t, 1H), 6.54 (m, 2H), 6.25 (m, 1H), 3.95/3.82 (dd+dd, 2H), 2.99/2.52 (dd+dd, 2H), 2.97 (m, 1H), 2.67/2.36 (dm+dd, 2H), 2.48-1.31 (m, 8H), 2.13 (m, 1H), 2.02/0.96 (m+m, 2H), 2.00 (m, 1H), 1.62 (m, 1H), 1.46/1.36 (m+m, 2H), 1.14 (d, 3H), 1.04 (d, 3H), 1.01 (d, 3H). HRMS calculated for C₃₆H₄₃N₂O₃Cl: 586.2962; found: 587.3014 (M+H).

### Example 666 (1r,2′S,4S)-4-(3-chloroanilino)-2′-[(2R)-3-{[(5S,7S)-5,7-dimethyl-5,6,7,8-tetrahydroquinolin-4-yl]oxy}-2-methylpropyl]-2′,3′-dihydrospiro[cyclohexane-1,1′-indene]-4-carboxylic acid

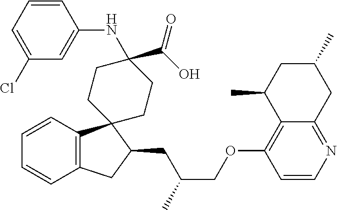 [SMILES: C[C@@H]1Cc2nccc(OC[C@H](C)C[C@H]3Cc4ccccc4[C@]34CC[C@@](Nc3cccc(Cl)c3)(C(=O)O)CC4)c2[C@@H](C)C1]

Using General procedure 32 and Preparation 10b1 as the appropriate indane and Example 664D as the appropriate alcohol Example 666 was obtained. ¹H NMR (500 MHz, DMSO-d₆) δ ppm: 12.67 (br s, 1H), 8.15 (d, 1H), 7.40-7.10 (m, 4H), 7.05 (t, 1H), 6.76 (d, 1H), 6.63 (t, 1H), 6.54 (dm, 1H), 6.52 (dm, 1H), 6.22 (m, 1H), 3.92/3.84 (dd+dd, 2H), 3.04 (m, 1H), 3.01/2.55 (dd+dd, 2H), 2.83/2.25 (dd+dd, 2H), 2.48-1.35 (m, 8H), 2.15 (m, 1H), 2.06-1.94 (m, 2H), 1.57/1.34 (m+m, 2H), 1.49-1.32 (m, 2H), 1.14 (d, 3H), 1.05 (d, 3H), 1.02 (d, 3H). HRMS calculated for C₃₆H₄₃N₂O₃Cl: 586.2962; found: 587.2962 (M+H).

### Example 667 (1r,2′S,4S)-4-(3-chloroanilino)-2′-[(2R)-3-{[(5R,7R)-5,7-dimethyl-5,6,7,8-tetrahydroquinolin-4-yl]oxy}-2-methylpropyl]-2′,3′-dihydrospiro[cyclohexane-1,1′-indene]-4-carboxylic acid

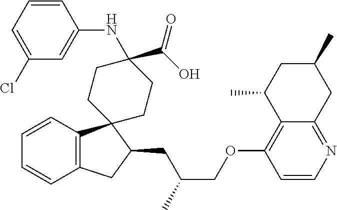 [SMILES: C[C@H]1Cc2nccc(OC[C@H](C)C[C@H]3Cc4ccccc4[C@]34CC[C@@](Nc3cccc(Cl)c3)(C(=O)O)CC4)c2[C@H](C)C1]

Using General procedure 32 and Preparation 10b1 as the appropriate indane and Example 664E as the appropriate alcohol Example 667 was obtained. ¹H NMR (500 MHz, DMSO-d₆) δ ppm: 12.67 (br s, 1H), 8.15 (d, 1H), 7.40-7.11 (m, 4H), 7.04 (t, 1H), 6.77 (d, 1H), 6.61 (t, 1H), 6.53 (m, 2H), 6.24 (m, 1H), 3.91/3.86 (dd+dd, 2H), 3.07 (m, 1H), 3.00/2.53 (dd+dd, 2H), 2.83/2.25 (dd+dd, 2H), 2.48-1.32 (m, 8H), 2.13 (m, 1H), 2.06-1.94 (m, 1H), 2.00 (m, 1H), 1.56/1.38 (d+m, 2H), 1.48/1.35 (m+m, 2H), 1.05 (d, 3H), 1.05 (d, 3H), 1.02 (d, 3H). HRMS calculated for C₃₆H₄₃N₂O₃Cl: 586.2962; found: 587.3018 (M+H).

### Example 668 and Example 669

### Example 668A (8R)-8-{[tert-butyl(dimethyl)silyl]oxy}-4-chloro-5,6,7,8-tetrahydroquinoline

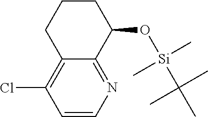 [SMILES: CC(C)(C)[Si](C)(C)O[C@@H]1CCCc2c(Cl)ccnc21]

and

### Example 669A (8S)-8-{[tert-butyl(dimethyl)silyl]oxy}-4-chloro-5,6,7,8-tetrahydroquinoline

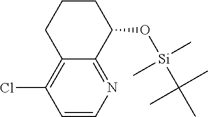 [SMILES: CC(C)(C)[Si](C)(C)O[C@H]1CCCc2c(Cl)ccnc21]

To a solution of 4-chloro-5,6,7,8-tetrahydro-quinolin-8-ol (440 mg, 2.4 mmol, 1 eq) and DIPEA (1.19 mL, 7.19 mmol, 3 eq) in DCM (10 mL), cooled to 0° C., was added TBDMS-OTf (1.38 mL, 5.99 mmol, 2.5 eq). The mixture was stirred at 0° C. for 1 h and then at rt for 1 h. The mixture was partitioned between DCM and water, and the organic phase was washed with brine, dried (PTFE phase separator) and concentrated in vacuo. Purification by automated flash chromatography (CombiFlash Rf, Silica 24 g RediSep™ cartridge) eluting with a gradient of 0-17% EtOAc in heptane afforded a racemate. The enantiomers were separated by chiral chromatography. Column: OD, 100 mm×500 mm, 20 m, Eluent: heptane. The enantiomer eluting earlier was collected as Example 668A, isolated as a colourless oil (297 mg, 1.0 mmol, 42%). ¹H NMR (400 MHz, DMSO-d₆) δ ppm: 8.40-8.37 (m, 1H), 7.44 (d, J=5.2 Hz, 1H), 4.77 (t, J=3.7 Hz, 1H), 2.90-2.80 (m, 1H), 2.66-2.54 (m, 1H), 2.04-1.74 (m, 4H), 0.85 (s, 9H), 0.19 (s, 3H), 0.02 (s, 3H). HRMS calculated for C₁₅H₂₄ClNOSi: 297.1316; found: 298.1386 (M+H).

The enantiomer eluting later was collected as Example 669A, isolated as a colourless oil (300 mg, 1.0 mmol, 43%). HRMS calculated for C₁₅H₂₄ClNOSi: 297.1316; found: 298.1389 (M+H).

### Example 668B methyl (1r,2′S,4S)-2′-[(2R)-3-{[(8R)-8-{[tert-butyl(dimethyl)silyl]oxy}-5,6,7,8-tetrahydroquinolin-4-yl]oxy}-2-methylpropyl]-4-(3-chloroanilino)-2′,3′-dihydrospiro[cyclohexane-1,1′-indene]-4-carboxylate

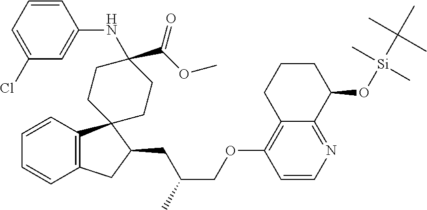 [SMILES: COC(=O)[C@]1(Nc2cccc(Cl)c2)CC[C@@]2(CC1)c1ccccc1C[C@@H]2C[C@@H](C)COc1ccnc2c1CCC[C@H]2O[Si](C)(C)C(C)(C)C]

Using General procedure 31a and Preparation 10b1 (120 mg, 0.27 mmol, 1 eq) as the appropriate alcohol and Example 668A (97 mg, 0.33 mmol, 1.2 eq) as the appropriate aryl chloride, Example 668B was obtained as an off-white glass (74 mg, 0.11 mmol, 39%). LRMS calculated for C₄₁H₅₅ClN₂O₄Si: 702; found: 703 (M+H).

### Example 668 (1r,2′S,4S)-4-(3-chloroanilino)-2′-[(2R)-3-{[(8R)-8-hydroxy-5,6,7,8-tetrahydroquinolin-4-yl]oxy}-2-methylpropyl]-2′,3′-dihydrospiro[cyclohexane-1,1′-indene]-4-carboxylic acid

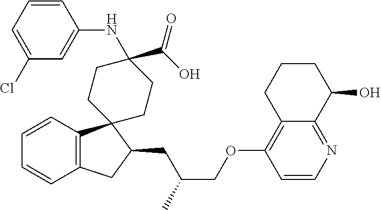 [SMILES: C[C@@H](COc1ccnc2c1CCC[C@H]2O)C[C@H]1Cc2ccccc2[C@]12CC[C@@](Nc1cccc(Cl)c1)(C(=O)O)CC2]

To a solution of Example 668B (74 mg, 0.11 mmol, 1 eq) in 1,4-dioxane (2 mL) was added 4 M HCl solution in 1,4-dioxane (0.5 mL, 2 mmol, 19 eq). The mixture was stirred at rt for 18 h. The mixture was partitioned between DCM and 2 M aq. NaOH solution, and the organic phase was washed with brine, dried (MgSO₄) and concentrated in vacuo. Purification by automated flash chromatography (CombiFlash Rf, Silica 4 g RediSep™ cartridge) eluting with a gradient of 0-10% MeOH in DCM afforded an intermediate that was hydrolyzed according to General procedure 33b to afford Example 668, isolated as a white powder (12.7 mg, 0.02 mmol, 21%). LRMS calculated for C₃₄H₃₉ClN₂O₄: 574; found: 575 (M+H). ¹H NMR (400 MHz, DMSO-d₆) δ ppm: 8.26 (d, J=5.6 Hz, 1H), 7.41-7.35 (m, 1H), 7.24-7.12 (m, 3H), 7.05 (t, J=8.0 Hz, 1H), 6.86 (d, J=5.6 Hz, 1H), 6.62 (t, J=2.1 Hz, 1H), 6.58-6.52 (m, 2H), 5.03-4.93 (br m, 1H), 4.53-4.46 (m, 1H), 3.95-3.86 (m, 2H), 3.00 (dd, J=15.6, 7.1 Hz, 1H), 2.67-2.35 (m, 4H), 2.20-2.06 (m, 2H), 2.06-1.57 (m, 9H), 1.54-1.31 (m, 4H), 1.04 (d, J=6.6 Hz, 3H).

### Example 669B methyl (1r,2′S,4S)-2′-[(2R)-3-{[(8S)-8-{[tert-butyl(dimethyl)silyl]oxy}-5,6,7,8-tetrahydroquinolin-4-yl]oxy}-2-methylpropyl]-4-(3-chloroanilino)-2′,3′-dihydrospiro[cyclohexane-1,1′-indene]-4-carboxylate

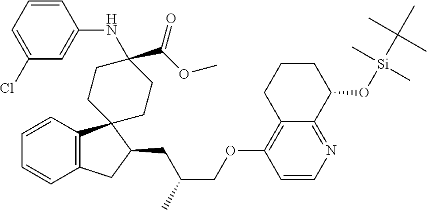 [SMILES: COC(=O)[C@]1(Nc2cccc(Cl)c2)CC[C@@]2(CC1)c1ccccc1C[C@@H]2C[C@@H](C)COc1ccnc2c1CCC[C@@H]2O[Si](C)(C)C(C)(C)C]

Using General procedure 31a and Preparation 10b1 (120 mg, 0.27 mmol, 1 eq) as the appropriate alcohol and Example 669A (97 mg, 0.33 mmol, 1.2 eq) as the appropriate aryl chloride, Example 669B was obtained as an off-white glass (90 mg, 0.13 mmol, 47%). LRMS calculated for C₄₁H₅₅ClN₂O₄Si: 702; found: 703 (M+H).

### Example 669 (1r,2′S,4S)-4-(3-chloroanilino)-2′-[(2R)-3-{[(8S)-8-hydroxy-5,6,7,8-tetrahydroquinolin-4-yl]oxy}-2-methylpropyl]-2′,3′-dihydrospiro[cyclohexane-1,1′-indene]-4-carboxylic acid

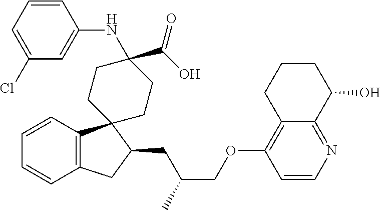 [SMILES: C[C@@H](COc1ccnc2c1CCC[C@@H]2O)C[C@H]1Cc2ccccc2[C@]12CC[C@@](Nc1cccc(Cl)c1)(C(=O)O)CC2]

To a solution of Example 669B (90 mg, 0.13 mmol, 1 eq) in 1,4-dioxane (2 mL) was added 4 M HCl solution in 1,4-dioxane (0.5 mL, 2 mmol, 19 eq). The mixture was stirred at rt for 18 h and then partitioned between DCM and 2 M aq. NaOH solution. The phases were separated, and the organic phase was washed with brine, dried (MgSO₄) and concentrated in vacuo. Purification by automated flash chromatography (CombiFlash Rf, Silica 4 g RediSep™ cartridge) eluting with a gradient of 0-10% MeOH in DCM afforded an intermediate which was hydrolyzed according to General procedure 33b, to afford Example 669, isolated as a white powder (16.6 mg, 0.03 mmol, 23%). LRMS calculated for C₃₄H₃₉ClN₂O₄: 574; found: 575 (M+H). ¹H NMR (400 MHz, DMSO-d₆) δ ppm: 8.26 (d, J=5.6 Hz, 1H), 7.42-7.34 (m, 1H), 7.24-7.11 (m, 3H), 7.04 (t, J=8.0 Hz, 1H), 6.86 (d, J=5.6 Hz, 1H), 6.63 (t, J=2.1 Hz, 1H), 6.59-6.50 (m, 2H), 5.07-4.87 (br m, 1H), 4.54-4.44 (m, 1H), 3.95-3.85 (m, 2H), 3.00 (dd, J=15.6, 7.1 Hz, 1H), 2.64-2.37 (m, 4H), 2.19-2.06 (m, 2H), 2.05-1.94 (m, 2H), 1.94-1.60 (m, 7H), 1.54-1.30 (m, 4H), 1.04 (d, J=6.6 Hz, 3H).

### Example 670

### Example 670A 4-chloro-6-methyl-1-oxo-5,6,7,8-tetrahydro-1λ⁵-quinoline

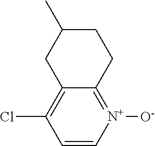

To a solution of Example 651C (1.74 g, 9.58 mmol, 1 eq) in DCM (50 mL) was added mCPBA (75% purity, 2.64 g, 11.5 mmol, 1.2 eq) portionwise and the mixture was stirred at rt for 18 h. 2 M aq. NaOH solution (50 mL) was added slowly and the mixture was stirred at rt for 30 min, then partitioned between DCM and water. The phases were separated and the organic phase was washed with brine, dried (MgSO₄) and concentrated in vacuo to afford a racemate, Example 670A, isolated as a yellow solid (1.78 g, 9.02 mmol, 94%) that was used directly in the subsequent step without further purification. LRMS calculated for C₁₀H₁₂ClNO: 197; found: 198 (M+H). ¹H NMR (400 MHz, DMSO-d₆) δ ppm: 8.16 (d, J=6.9 Hz, 1H), 7.44-7.39 (m, 1H), 3.01-2.90 (m, 1H), 2.90-2.80 (m, 1H), 2.64-2.52 (m, 1H), 2.30-2.19 (m, 1H), 1.97-1.87 (m, 1H), 1.86-1.72 (m, 1H), 1.43-1.30 (m, 1H), 1.06 (d, J=6.6 Hz, 3H).

### Example 670B rac-(6R,8S)-4-chloro-6-methyl-5,6,7,8-tetrahydroquinolin-8-ol

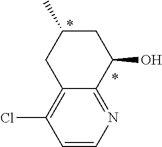

To a solution of Example 670A (1.78 g, 9.01 mmol, 1 eq) in DCM (40 mL) was added TFAA (3.76 mL, 27 mmol, 3 eq) and the mixture was stirred at rt for 18 h. A mixture of cis and trans isomers was formed. The mixture was quenched with 2 M aq. NaOH solution and partitioned between DCM/iPrOH (5:1) and water. The phases were separated, and the organic phase was washed with brine, dried (MgSO₄) and concentrated in vacuo. The diastereoisomers were separated by automated flash chromatography (CombiFlash Rf, Silica 24 g RediSep™ cartridge) eluting with a gradient of 25-90% EtOAc in heptane. The racemic diastereoisomer eluting later was collected as Example 670B, isolated as a white powder (381 mg, 1.93 mmol, 21%). LRMS calculated for C₁₀H₁₂ClNO: 197; found: 198 (M+H). ¹H NMR (400 MHz, DMSO-d₆) δ ppm: 8.40-8.35 (m, 1H), 7.43 (d, J=5.2 Hz, 1H), 5.34 (d, J=4.7 Hz, 1H), 4.63-4.58 (m, 1H), 3.00-2.87 (m, 1H), 2.27-2.12 (m, 2H), 1.96-1.87 (m, 1H), 1.59-1.46 (m, 1H), 1.09 (d, J=6.2 Hz, 3H).

### Example 670C rac-(6R,8S)-4-chloro-8-[(4-methoxyphenyl)methoxy]-6-methyl-5,6,7,8-tetrahydroquinoline

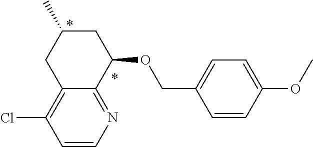 [SMILES: COc1ccc(CO[C@@H]2C[C@@H](C)Cc3c(Cl)ccnc32)cc1]

NaH (60% dispersion; 154 mg, 3.86 mmol, 2 eq) was added to a solution of Example 670B (381 mg, 1.93 mmol, 1 eq), PMBCl (312 μL, 2.31 mmol, 1.2 eq) and TBAI (71 mg, 193 μmol, 0.1 eq) in DMF (7 mL) and stirred at rt under N₂ for 18 h. The mixture was partitioned between DCM and sat. aq. NaHCO₃ solution. The phases were separated and the organic phase was dried (PTFE phase separator) and concentrated in vacuo. Purification by automated flash chromatography (CombiFlash Rf, Silica 12 g RediSep™ cartridge) eluting with a gradient of 0-35% EtOAc in heptane afforded a racemate, Example 670C, isolated as a colourless oil. LRMS calculated for C₁₈H₂0ClNO₂: 317; found: 318 (M+H). ¹H NMR (400 MHz, DMSO-d₆) δ ppm: 8.44-8.41 (m, 1H), 7.50 (d, J=5.2 Hz, 1H), 7.29-7.22 (m, 2H), 6.92-6.86 (m, 2H), 4.67 (d, J=11.6 Hz, 1H), 4.61 (d, J=11.6 Hz, 1H), 4.47 (t, J=2.8 Hz, 1H), 3.74 (s, 3H), 3.04-2.92 (m, 1H), 2.26-2.08 (m, 3H), 1.55-1.45 (m, 1H), 1.10 (d, J=5.9 Hz, 3H).

### Example 670D (1r,2′S,4S)-4-(3-chloroanilino)-2′-[(2R)-3-({(6RS,8SR)-8-[(4-methoxyphenyl)methoxy]-6-methyl-5,6,7,8-tetrahydroquinolin-4-yl}oxy)-2-methylpropyl]-2′,3′-dihydrospiro[cyclohexane-1,1′-indene]-4-carboxylic acid

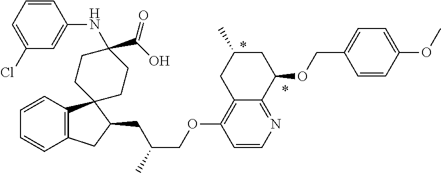 [SMILES: COc1ccc(CO[C@@H]2C[C@@H](C)Cc3c(OC[C@H](C)C[C@H]4Cc5ccccc5[C@]45CC[C@@](Nc4cccc(Cl)c4)(C(=O)O)CC5)ccnc32)cc1]

Using General procedure 31c and Preparation 10b1 (160 mg, 0.36 mmol, 1 eq) as the appropriate alcohol and Example 670C (253 mg, 0.8 mmol, 2.2 eq) as the appropriate aryl chloride, a mixture of diastereoisomers, Example 670D was isolated as a colourless glass (67 mg, 0.09 mmol, 26%). LRMS calculated for C₄₃H₄₉ClN₂O₅: 708; found: 709 (M+H). ¹H NMR (400 MHz, DMSO-d₆) δ ppm: 8.72 (d, J=6.7 Hz, 1H), 7.55-7.44 (m, 1H), 7.40-7.28 (m, 3H), 7.24-7.11 (m, 3H), 7.08-7.01 (m, 1H), 6.93-6.87 (m, 2H), 6.65-6.60 (m, 1H), 6.58-6.51 (m, 2H), 4.82-4.76 (m, 1H), 4.73-4.62 (m, 2H), 4.25-4.14 (m, 2H), 3.74/3.74 (s, 3H), 3.02 (dd, J=15.7, 7.0 Hz, 1H), 2.89-2.77 (m, 1H), 2.60-2.37 (m, 2H), 2.26-1.80 (m, 9H), 1.78-1.64 (m, 1H), 1.55-1.28 (m, 5H), 1.12-1.02 (m, 6H).

### Example 670 (1r,2′S,4S)-4-(3-chloroanilino)-2′-[(2R)-3-{[(6RS,8SR)-8-hydroxy-6-methyl-5,6,7,8-tetrahydroquinolin-4-yl]oxy}-2-methylpropyl]-2′,3′-dihydrospiro[cyclohexane-1,1′-indene]-4-carboxylic acid

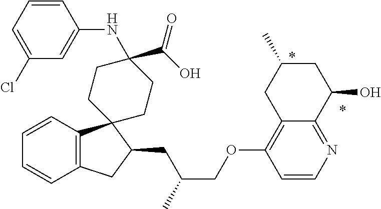 [SMILES: C[C@H]1Cc2c(OC[C@H](C)C[C@H]3Cc4ccccc4[C@]34CC[C@@](Nc3cccc(Cl)c3)(C(=O)O)CC4)ccnc2[C@H](O)C1]

Using General procedure 28b and Example 670D (67 mg, 0.09 mmol, 1 eq) as the appropriate PMB derivative and then purification by reverse phase automated flash chromatography (CombiFlash Rf, C18 5.5 g Gold RediSep column) eluting with a gradient of 10-75% MeCN in water, a mixture of 2 diastereoisomers, Example 670 was isolated as a white powder (17.9 mg, 0.03 mmol, 32%). LRMS calculated for C₃₅H₄₁ClN₂O₄: 588; found: 589 (M+H). ¹H NMR (400 MHz, DMSO-d₆) δ ppm: 8.26 (d, J=5.6 Hz, 1H), 7.41-7.32 (m, 1H), 7.24-7.11 (m, 3H), 7.05 (t, J=8.1 Hz, 1H), 6.89-6.83 (m, 1H), 6.65-6.61 (m, 1H), 6.59-6.52 (m, 2H), 5.08-5.02 (m, 1H), 4.54-4.49 (m, 1H), 3.94-3.83 (m, 2H), 3.01 (dd, J=15.6, 7.0 Hz, 1H), 2.82-2.70 (m, 1H), 2.60-2.38 (m, 2H), 2.22-1.67 (m, 10H), 1.54-1.26 (m, 5H), 1.08-0.98 (m, 6H).

### Example 671 and Example 672

### Example 671A 2,2-dimethyl-5-{[(3-oxocyclohex-1-en-1-yl)amino]methylidene}-1,3-dioxane-4,6-dione

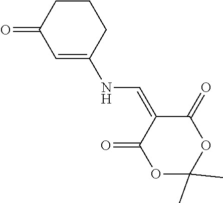 [SMILES: CC1(C)OC(=O)C(=CNC2=CC(=O)CCC2)C(=O)O1]

5-(Methoxymethylene)-2,2-dimethyl-1,3-dioxane-4,6-dione (12.06 g, 64.78 mmol, 1.2 eq) was slowly added to a stirred solution of 3-aminocyclohex-2-en-1-one (6.00 g, 53.99 mmol, 1 eq) in MeCN (60 mL) and the suspension was heated at 70° C. for 4 h and then stirred at rt for 12 h. The suspension was filtered, the solids were washed with MeOH and dried in vacuo to afford Example 671A as a cream powder (4.37 g, 16.47 mmol, 31%). The filtrate was concentrated in vacuo and triturated with MeOH and the solids were collected by filtration and washed with MeOH and dried to afford additional Example 671A as a yellow powder (1.8 g, 6.79 mmol, 13%). ¹H NMR (400 MHz, DMSO-d₆) δ ppm: 10.92 (d, J=14.0 Hz, 1H), 8.34 (d, J=14.0 Hz, 1H), 6.15-6.10 (m, 1H), 2.72 (t, J=6.1 Hz, 2H), 2.32-2.25 (m, 2H), 2.03-1.94 (m, 2H), 1.68 (s, 6H).

### Example 671B 4-chloro-7,8-dihydroquinolin-5 (6H)-one

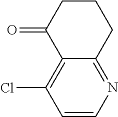 [SMILES: O=C1CCCc2nccc(Cl)c21]

Example 671A (4.37 g, 16.47 mmol, 1 eq) was added to degassed Dowtherm A (24 mL) at rt and then the mixture was placed in a hotplate, pre-heated to 180° C., and then heated at 215° C. for 30 min. The mixture was allowed to cool to rt, diluted with DCM/heptane (3:1) and loaded onto a silica pad, washed successively with heptane, 50% EtOAc in heptane, EtOAc, 10% MeOH in DCM and 12% MeOH in DCM (3% TEA). The solvents were removed in vacuo and triturated with Et₂O and EtOAc. The intermediate was cooled to 0° C., POCl₃ (13 mL, 140.34 mmol, 10 eq) was added slowly and then the cooling bath was removed, and the mixture was heated at 60° C. with stirring for 2 h. The mixture was allowed to cool to rt and quenched by the dropwise addition to rapidly stirred ice-water. The mixture was stirred for 15 min and then basified with aq. NH₃ solution. The aq. phase was extracted with 10% MeOH in DCM, and the combined organic extracts were dried (MgSO₄) and concentrated in vacuo. Purification by automated flash chromatography (Combiflash Rf, Silica 40 g RediSep™ cartridge) eluting with a gradient of 0-50% EtOAc (3% TEA) in heptane afforded Example 671B as an orange oil (587 mg, 3.23 mmol, 23%). LRMS calculated for C₉H₈ClNO: 181; found: 182 (M+H). ¹H NMR (400 MHz, DMSO-d₆) δ ppm: 8.55 (d, J=5.3 Hz, 1H), 7.52-7.48 (m, 1H), 3.15-3.08 (m, 2H), 2.71-2.65 (m, 2H), 2.11-2.02 (m, 2H).

### Example 671C 4-chloro-5,6,7,8-tetrahydroquinolin-5-ol

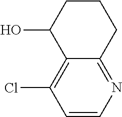 [SMILES: OC1CCCc2nccc(Cl)c21]

To a solution of Example 671B (587 mg, 3.23 mmol, 1 eq) in MeOH (15 mL), cooled to 0° C., was added NaBH₄ (183 mg, 4.85 mmol, 1.5 eq) and the mixture was allowed to warm to rt and stirred under N₂ for 3 h. The solvent was removed in vacuo and the residue was loaded on to a pre-wetted (MeOH) 10 g SCX-2 cartridge, washed with DCM, MeOH and eluting with 4:1 DCM/MeOH (10% TEA). The solvents were removed in vacuo to afford a racemate, Example 671C, isolated as a yellow solid (516 mg, 2.81 mmol, 87%). LRMS calculated for C₉H₁₀ClNO: 183; found: 184 (M+H). ¹H NMR (400 MHz, DMSO-d₆) δ ppm: 8.34 (d, J=5.2 Hz, 1H), 7.37-7.34 (m, 1H), 5.18 (d, J=5.8 Hz, 1H), 4.92-4.86 (m, 1H), 2.94-2.85 (m, 1H), 2.79-2.68 (m, 1H), 2.10-1.91 (m, 2H), 1.80-1.62 (m, 2H).

### Example 671D 4-chloro-5-methoxy-5,6,7,8-tetrahydroquinoline

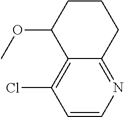

NaH (60% dispersion; 72 mg, 1.8 mmol, 1.1 eq) was added to a solution of Example 671C (300 mg, 1.63 mmol, 1 eq) in THE (5 mL), cooled to 0° C. under N₂ and the mixture was stirred for 5 min. Mel (107 μL, 1.72 mmol, 1.05 eq) was added and the mixture was allowed to warm to rt and stirred for 2 h. DMF (2 mL) and NaH (36 mg, 0.9 mmol, 0.55 eq) followed by Mel (53 μL, 0.86 mmol, 0.5 eq) were added and the mixture was stirred at rt for 18 h. The mixture was partitioned between DCM and sat. aq. NaHCO₃ solution and the organic phase was dried (PTFE phase separator) and concentrated in vacuo. Purification by automated flash chromatography (Combiflash Rf, Silica 12 g RediSep™ cartridge) eluting with a gradient of 0-50% EtOAc (3% TEA) in heptane afforded a racemate, Example 671D, isolated as a yellow oil (238 mg, 1.2 mmol, 74%). LRMS calculated for C₁₀H₁₂ClNO: 197; found: 198 (M+H). ¹H NMR (400 MHz, DMSO-d₆) δ ppm: 8.38 (d, J=5.2 Hz, 1H), 7.42-7.37 (m, 1H), 4.50-4.45 (m, 1H), 3.39 (s, 3H), 2.95-2.85 (m, 1H), 2.82-2.69 (m, 1H), 2.31-2.22 (m, 1H), 1.90-1.73 (m, 2H), 1.60-1.49 (m, 1H).

### Example 671 (1r,2′S,4S)-4-(3-chloroanilino)-2′-[(2R)-3-{[(5S)-5-methoxy-5,6,7,8-tetrahydroquinolin-4-yl]oxy}-2-methylpropyl]-2′,3′-dihydrospiro[cyclohexane-1,1′-indene]-4-carboxylic acid

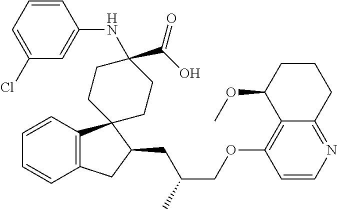 [SMILES: CO[C@H]1CCCc2nccc(OC[C@H](C)C[C@H]3Cc4ccccc4[C@]34CC[C@@](Nc3cccc(Cl)c3)(C(=O)O)CC4)c21]

and

### Example 672 (1r,2′S,4S)-4-(3-chloroanilino)-2′-[(2R)-3-{[(5R)-5-methoxy-5,6,7,8-tetrahydroquinolin-4-yl]oxy}-2-methylpropyl]-2′,3′-dihydrospiro[cyclohexane-1,1′-indene]-4-carboxylic acid

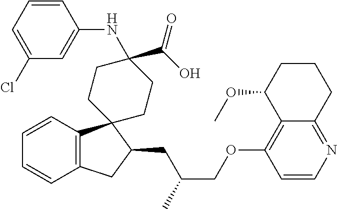 [SMILES: CO[C@@H]1CCCc2nccc(OC[C@H](C)C[C@H]3Cc4ccccc4[C@]34CC[C@@](Nc3cccc(Cl)c3)(C(=O)O)CC4)c21]

Using General procedure 31c and Preparation 10b1 as the appropriate indane and Example 671D as the appropriate aryl chloride, a mixture of diastereoisomers was obtained. The diastereoisomers were separated by chiral chromatography. Column: AD, 50×500 mm, 20 μm, Eluents: MeOH/iPrOH/heptane. The diastereoisomer eluting earlier was collected as Example 671. ¹H NMR (500 MHz, DMSO-d₆) δ ppm: 12.70 (br s, 1H), 8.45 (d, 1H), 7.41-7.10 (m, 4H), 7.14 (d, 1H), 7.05 (t, 1H), 6.62 (t, 1H), 6.55 (dm, 1H), 6.53 (dm, 1H), 6.22 (br s, 1H), 4.38 (m, 1H), 4.11/3.98 (dd+dd, 2H), 3.27 (s, 3H), 3.00/2.56 (dd+dd, 2H), 2.94-1.28 (m, 17H), 2.17 (m, 1H), 1.07 (d, 3H). HRMS calculated for C₃₅H₄₁N₂O₄Cl: 588.2755; found: 589.2811 (M+H).

The diastereoisomer eluting later was collected as Example 672. ¹H NMR (500 MHz, DMSO-d₆) δ ppm: 12.73 (br s, 1H), 8.55 (d, 1H), 7.41-7.10 (m, 4H), 7.29 (d, 1H), 7.06 (t, 1H), 6.62 (t, 1H), 6.55 (dm, 1H), 6.25 (br s, 1H), 6.24 (dm, 1H), 4.42 (m, 1H), 4.17/4.08 (dd+dd, 2H), 3.17 (s, 3H), 3.02/2.56 (dd+dd, 2H), 2.98-1.30 (m, 17H), 2.18 (m, 1H), 1.07 (d, 3H). HRMS calculated for C₃₅H₄₁N₂O₄Cl: 588.2755; found: 589.2832 (M+H).

### Example 673 and Example 674 and Example 675 and Example 676

### Example 673A 5-methyl-5,6,7,8-tetrahydroquinoline

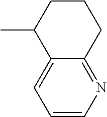

PtO₂ (476 mg, 2.1 mmol, 0.05 eq) was added to a solution of 5-methylquinoline (6.00 g, 41.9 mmol, 1 eq) in TFA (100 mL, 1341.83 mmol, 32 eq) under a N₂ atmosphere. The mixture was evacuated and backfilled with N₂ (×3), then evacuated and backfilled with H₂ and heated at 52° C., with stirring, under an atmosphere of H₂ for 10 h. The mixture was allowed to cool to rt, filtered through celite, washed with minimal MeOH and the solvents were removed in vacuo. The residue was dissolved in DCM and washed with 2 M aq. NaOH solution, then the organic phase was dried (MgSO₄) and concentrated in vacuo. Purification by automated flash chromatography (Combiflash Rf, 80 g RediSep™ silica cartridge) eluting with a gradient of 0-27% EtOAc (3% TEA) in heptane afforded a racemate, Example 673A, isolated as a colourless oil (3.59 g, 24.36 mmol, 58%). LRMS calculated for C₁₀H₁₃N: 147; found: 148 (M+H). ¹H NMR (400 MHz, DMSO-d₆) δ ppm: 8.31-8.25 (m, 1H), 7.62-7.57 (m, 1H), 7.16-7.11 (m, 1H), 2.97-2.72 (m, 3H), 1.95-1.82 (m, 2H), 1.81-1.69 (m, 1H), 1.55-1.43 (m, 1H), 1.24 (d, J=7.0 Hz, 3H).

### Example 673B 5-methyl-1-oxo-5,6,7,8-tetrahydro-1λ⁵-quinoline

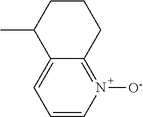

mCPBA (77% purity, 6.01 g, 26.8 mmol, 1.1 eq) was added portionwise over 45 min to a stirred solution of Example 673A (3.59 g, 24.4 mmol, 1 eq) in DCM (100 mL) at 0° C. and then the mixture was stirred at rt for 4 h. Sat. aq. NaHCO₃ solution (50 mL) was added and the mixture was stirred for 30 min. 2 M aq. NaOH solution was added, and the layers were separated. The aq. phase was extracted with DCM and the combined organic extracts were washed with a 1:1 mix of 2 M aq. NaOH solution and sat. aq. NaHCO₃ solution. The organic phase was dried (MgSO₄) and concentrated in vacuo to afford a racemate, Example 673B, isolated as a yellow solid (3.68 g, 22.55 mmol, 92%). LRMS calculated for C₁₀H₁₃NO: 163; found: 164 (M+H). ¹H NMR (400 MHz, DMSO-d₆) δ ppm: 8.16-8.09 (m, 1H), 7.27-7.18 (m, 2H), 2.98-2.87 (m, 1H), 2.80-2.63 (m, 2H), 1.93-1.69 (m, 3H), 1.52-1.42 (m, 1H), 1.25 (d, J=7.0 Hz, 3H).

### Example 673C 4-chloro-5-methyl-5,6,7,8-tetrahydroquinoline

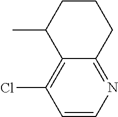

To stirring POCl₃ (21 mL, 34.6 g, 225 mmol, 10 eq) at 0° C. was added Example 673B (3.68 g, 22.55 mmol) in portions over 25 min. On complete addition, the temperature was gradually increased, and the mixture was stirred at 60° C. until no further conversion was observed. A mixture of regioisomers was obtained. The mixture was allowed to cool to rt and then quenched by the dropwise addition to rapidly stirred ice-water. The pH was adjusted to 12 by the dropwise addition of aq. cc. NH₃ solution and the mixture was extracted with DCM. The combined organic extracts were washed with brine, dried (MgSO₄) and concentrated in vacuo. The regioisomers were separated by automated flash chromatography (Combiflash Rf, 80 g RediSep™ silica cartridge) eluting with a gradient of 0-20% EtOAc in heptane. The regioisomer eluting later was collected and further purified by automated flash chromatography (Combiflash Rf, Gold 40 g RediSep™ silica cartridge) eluting with a gradient of 0-25% EtOAc in DCM to obtain Example 673C, as a racemate, isolated as a colourless oil (1.68 g, 9.26 mmol, 41%). LRMS calculated for C₁₀H₁₂NCl: 181; found: 182 (M+H). ¹H NMR (400 MHz, DMSO-d₆) δ ppm: 8.27 (d, J=5.2 Hz, 1H), 7.33-7.29 (m, 1H), 3.26-3.16 (m, 1H), 2.96-2.86 (m, 1H), 2.84-2.71 (m, 1H), 2.01-1.70 (m, 4H), 1.21 (d, J=7.0 Hz, 3H).

### Example 673D 4-chloro-5-methyl-1-oxo-5,6,7,8-tetrahydro-1λ⁵-quinoline

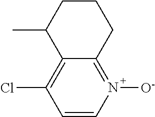

mCPBA (70% purity; 2.07 g, 8.39 mmol, 1.2 eq) was added portionwise to a solution of Example 673C (1.27 g, 6.99 mmol, 1 eq) in DCM (100 mL) cooled to 0° C. The mixture was allowed to warm to rt and stirred for 18 h. 2 M aq. NaOH solution was added and the mixture was stirred for 20 min, extracted with DCM and the combined organic extracts were dried (MgSO₄) and concentrated in vacuo to afford a racemate, Example 673D, isolated as a yellow oil (1.46 g, 7.39 mmol). LRMS calculated for C₁₀H₁₂ClNO: 197; found: 198 (M+H). ¹H NMR (400 MHz, DMSO-d₆) δ ppm: 8.17 (d, J=6.9 Hz, 1H), 7.44-7.39 (m, 1H), 3.21-3.11 (m, 1H), 3.00-2.89 (m, 1H), 2.53-2.42 (m, 1H), 1.94-1.60 (m, 4H), 1.21 (d, J=7.0 Hz, 3H).

### Example 673E rac-(5R,8S)-4-chloro-5-methyl-5,6,7,8-tetrahydroquinolin-8-ol

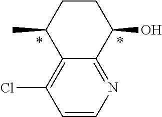

and

### Example 675A rac-(5R,8R)-4-chloro-5-methyl-5,6,7,8-tetrahydroquinolin-8-ol

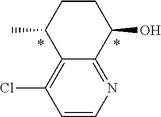

TFAA (2.96 mL, 21.25 mmol, 3 eq) was added dropwise to a solution of Example 673D (1.4 g, 7.08 mmol, 1 eq) in DCM (20 mL), cooled to 0° C. under N₂, and the mixture was allowed to warm to rt and stirred for 18 h. A racemic mixture of diastereoisomers was obtained. 2 M aq. NaOH solution was added, and the mixture was stirred for 2 h and then extracted with DCM. The organic phase was dried (PTFE phase separator) and concentrated in vacuo. The diastereoisomers were separated by automated flash chromatography (Combiflash Rf, 24 g RediSep™ silica cartridge) eluting with a gradient of 0-65% EtOAc (3% TEA) in heptane. The racemic diastereoisomer eluting earlier was collected as Example 673E, isolated as a white solid (472 mg, 2.39 mmol, 34%). LRMS calculated for C₁₀H₁₂ClNO: 197; found 198 (M+H). ¹H NMR (400 MHz, DMSO-d₆) δ ppm: 8.38 (d, J=5.2 Hz, 1H), 7.43 (d, J=5.2 Hz, 1H), 5.21 (d, J=4.5 Hz, 1H), 4.58-4.53 (m, 1H), 3.24-3.15 (m, 1H), 2.21-2.01 (m, 2H), 1.87-1.79 (m, 1H), 1.57-1.49 (m, 1H), 1.18 (d, J=7.0 Hz, 3H).

The racemic diastereoisomer eluting later was collected as Example 675A, isolated as a colourless oil (548 mg, 2.77 mmol, 39%). LRMS calculated for C₁₀H₁₂ClNO: 197; found 198 (M+H). ¹H NMR (400 MHz, DMSO-d₆) δ ppm: 8.39 (d, J=5.2 Hz, 1H), 7.41 (dd, J=5.2, 0.7 Hz, 1H), 5.26 (d, J=3.8 Hz, 1H), 4.57-4.49 (m, 1H), 3.23-3.13 (m, 1H), 2.06-1.81 (m, 3H), 1.76-1.68 (m, 1H), 1.26 (d, J=7.0 Hz, 3H).

### Example 673 (1r,2′S,4S)-4-(3-chloroanilino)-2′-[(2R)-3-{[(5R,8S)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid

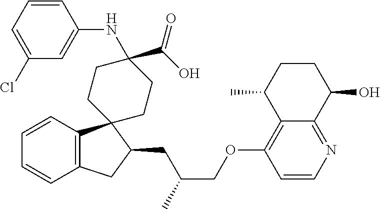 [SMILES: C[C@@H](COc1ccnc2c1[C@H](C)CC[C@H]2O)C[C@H]1Cc2ccccc2[C@]12CC[C@@](Nc1cccc(Cl)c1)(C(=O)O)CC2]

and

### Example 674 (1r,2′S,4S)-4-(3-chloroanilino)-2′-[(2R)-3-{[(5S,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid

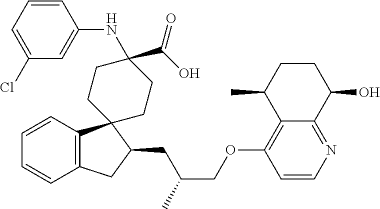 [SMILES: C[C@@H](COc1ccnc2c1[C@@H](C)CC[C@H]2O)C[C@H]1Cc2ccccc2[C@]12CC[C@@](Nc1cccc(Cl)c1)(C(=O)O)CC2]

Using General procedure 31a and Preparation 10b1 as the appropriate indane and Example 673E as the appropriate aryl chloride, a mixture of diastereoisomers was obtained. The diastereoisomers were separated by chiral chromatography. Column: AD, 50×500 mm, 20 μm, Eluents: iPrOH/heptane. The diastereoisomer eluting earlier was hydrolyzed according to General Procedure 33a to obtain Example 673. ¹H NMR (500 MHz, DMSO-d₆) δ ppm: 8.26 (d, 1H), 7.42-7.09 (m, 8H), 7.03 (t, 1H), 6.87 (d, 1H), 6.61 (t, 1H), 6.53 (m, 2H), 6.25 (br s, 1H), 4.92 (br, 1H), 4.43 (dd, 1H), 3.93/3.87 (dd+dd, 2H), 3.02 (m, 1H), 3.00/2.53 (dd+dd, 2H), 2.48-1.33 (m, 4H), 2.13 (m, 1H), 2.00 (m, 1H), 1.93/1.75 (m+m, 2H), 1.83/1.60 (m+m, 2H), 1.48/1.35 (m+m, 2H), 1.09 (d, 3H), 1.05 (d, 3H). HRMS calculated for C₃₅H₄₁N₂O₄Cl: 588.2755; found: 589.2820 (M+H).

The diastereoisomer eluting later was hydrolyzed according to General procedure 33a to obtain Example 674. ¹H NMR (500 MHz, DMSO-d₆) δ ppm: 12.75 (br s, 1H), 8.26 (d, 1H), 7.39-7.10 (m, 4H), 7.02 (t, 1H), 6.85 (d, 1H), 6.64 (t, 1H), 6.53 (dm, 1H), 6.51 (dm, 1H), 6.17 (br s, 1H), 4.92 (d, 1H), 4.41 (m, 1H), 3.94/3.85 (dd+dd, 2H), 3.01/2.56 (dd+dd, 2H), 2.98 (m, 1H), 2.50-1.10 (m, 15H), 2.15 (m, 1H), 1.18 (d, 3H), 1.05 (d, 3H). HRMS calculated for C₃₅H₄₁N₂O₄Cl: 588.2755; found: 589.2842 (M+H).

### Example 675 (1r,2′S,4S)-4-(3-chloroanilino)-2′-[(2R)-3-{[(5S,8S)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid

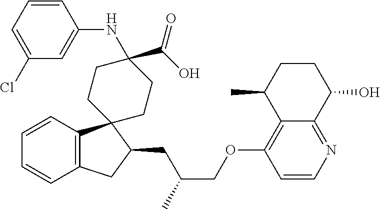 [SMILES: C[C@@H](COc1ccnc2c1[C@@H](C)CC[C@@H]2O)C[C@H]1Cc2ccccc2[C@]12CC[C@@](Nc1cccc(Cl)c1)(C(=O)O)CC2]

### Example 676 (1r,2′S,4S)-4-(3-chloroanilino)-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid

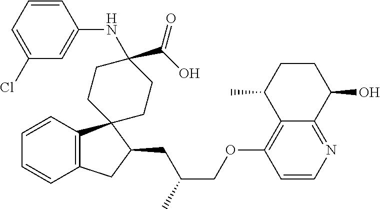 [SMILES: C[C@@H](COc1ccnc2c1[C@H](C)CC[C@H]2O)C[C@H]1Cc2ccccc2[C@]12CC[C@@](Nc1cccc(Cl)c1)(C(=O)O)CC2]

Using General procedure 31a and Preparation 10b1 as the appropriate indane and Example 675A as the appropriate aryl chloride, a mixture of diastereoisomers was obtained. The diastereoisomers were separated by chiral chromatography. Column: AD, 50×500 mm, 20 μm, Eluents: EtOH/heptane. The diastereoisomer eluting earlier was hydrolyzed according to General Procedure 33a to obtain Example 675. ¹H NMR (500 MHz, DMSO-d₆) δ ppm: 12.79 (br s, 1H), 8.24 (d, 1H), 7.35-7.13 (m, 4H), 7.01 (t, 1H), 6.84 (d, 1H), 6.64 (t, 1H), 6.54 (dd, 1H), 6.50 (m, 1H), 6.15 (br s, 1H), 4.91 (d, 1H), 4.44 (dm, 1H), 3.94/3.84 (dd+dd, 2H), 3.01/2.56 (dd+dd, 2H), 2.98 (m, 1H), 2.40-1.45 (m, 8H), 2.17 (m, 1H), 2.01 (m, 1H), 2.00/1.38 (m+m, 2H), 1.97/1.73 (m+m, 2H), 1.44-1.38 (m, 2H), 1.11 (d, 3H), 1.06 (d, 3H). HRMS calculated for C₃₅H₄₁N₂O₄Cl: 588.2755; found: 589.2831 (M+H).

The diastereoisomer eluting later was hydrolyzed according to General procedure 33a to obtain Example 676. ¹H NMR (500 MHz, DMSO-d₆) δ ppm: 12.76 (br s, 1H), 8.25 (d, 1H), 7.37 (dm, 1H), 7.21 (dm, 1H), 7.15-7.13 (m, 2H), 7.03 (t, 1H), 6.86 (d, 1H), 6.62 (t, 1H), 6.54 (dd, 1H), 6.52 (dd, 1H), 6.25 (br s, 1H), 4.93 (m, 1H), 4.44 (m, 1H), 3.92/3.84 (dd+dd, 2H), 3.03 (m, 1H), 3.01/2.54 (dd+dd, 2H), 2.44-1.39 (m, 8H), 2.14 (m, 1H), 2.05/1.38 (m+m, 2H), 2.02 (m, 1H), 1.96/1.73 (m+m, 2H), 1.48/1.36 (m+m, 2H), 1.05 (d, 3H), 1.01 (d, 3H). HRMS calculated for C₃₅H₄₁N₂O₄Cl: 588.2755; found: 589.2827 (M+H).

### Example 677 (1r,2′S,4S)-4-(3-chloroanilino)-2′-[(2R)-2-methyl-3-{[(5R)-5-methyl-5,6-dihydroquinolin-4-yl]oxy}propyl]-2′,3′-dihydrospiro[cyclohexane-1,1′-indene]-4-carboxylic acid

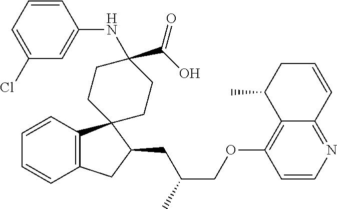 [SMILES: C[C@@H](COc1ccnc2c1[C@H](C)CC=C2)C[C@H]1Cc2ccccc2[C@]12CC[C@@](Nc1cccc(Cl)c1)(C(=O)O)CC2]

At the isolation of Example 689, Example 677 was also obtained. LRMS calculated for C₃₅H₃₉ClN₂O₃: 570; found: 571 (M+H). ¹H NMR (400 MHz, DMSO-d₆) δ ppm: 8.17 (d, J=5.8 Hz, 1H), 7.41-7.34 (m, 1H), 7.25-7.12 (m, 3H), 7.05 (t, J=8.1 Hz, 1H), 6.85 (d, J=5.8 Hz, 1H), 6.62 (t, J=2.1 Hz, 1H), 6.57-6.51 (m, 2H), 6.44 (dd, J=9.9, 3.0 Hz, 1H), 6.20-6.13 (m, 1H), 3.98-3.86 (m, 2H), 3.27-3.16 (m, 1H), 3.02 (dd, J=15.6, 7.0 Hz, 1H), 2.60-2.39 (m, 3H), 2.21-2.07 (m, 3H), 2.07-1.81 (m, 4H), 1.79-1.67 (m, 1H), 1.57-1.44 (m, 2H), 1.44-1.28 (m, 2H), 1.05 (d, J=6.7 Hz, 3H), 0.89 (d, J=7.0 Hz, 3H).

### Example 678 and Example 679

### Example 678A N-(3-ethylphenyl)acetamide

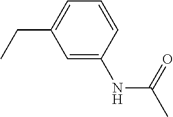

AcCl (3.24 mL, 45.39 mmol, 1.1 eq) was added dropwise to a solution of m-ethylaniline, (5.13 mL, 41.26 mmol, 1 eq) and TEA (11.47 mL, 82.52 mmol, 2 eq) in DCM (50 mL), cooled to 0° C. under N₂ and stirred for 2 h. The mixture was diluted with DCM and washed with water and sat. aq. NH₄Cl solution. The organic phase was dried (MgSO₄) and concentrated in vacuo to afford Example 678A as a brown oil (7.25 g, 44.42 mmol). LRMS calculated for C₁₀H₁₃NO: 163; found: 164 (M+H). ¹H NMR (400 MHz, DMSO-d₆) δ ppm: 9.86 (s, 1H), 7.45-7.36 (m, 2H), 7.19 (t, J=7.8 Hz, 1H), 6.90-6.85 (m, 1H), 2.56 (q, J=7.6 Hz, 2H), 2.03 (s, 3H), 1.16 (t, J=7.6 Hz, 3H).

### Example 678B N-(2-chloro-5-ethylphenyl)acetamide

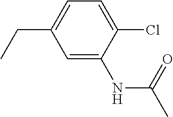

A mixture of Example 678A (525 mg, 3.22 mmol, 1 eq), PTSA (306 mg, 1.61 mmol, 0.5 eq), NCS (515 mg, 3.86 mmol, 1.2 eq) and Pd(OAc)₂ (36 mg, 0.16 mmol, 0.05 eq) in toluene (12 mL) was stirred under air at 50° C. for 3 h. The mixture was allowed to cool to rt and partitioned between EtOAc and sat. aq. NaHCO₃ solution. The organic phase was washed with water, dried (MgSO₄) and concentrated in vacuo. Purification by automated flash chromatography (Combiflash Rf, 12 g RediSep™ silica cartridge) eluting with a gradient of 0-40% EtOAc in heptane afforded Example 678B as a white powder (432 mg, 2.19 mmol, 68%). LRMS calculated for C₁₀H₁₂ClNO: 197; found: 198 (M+H). ¹H NMR (400 MHz, DMSO-d₆) δ ppm: 9.47 (s, 1H), 7.58-7.50 (m, 1H), 7.38 (d, J=8.2 Hz, 1H), 7.07-7.00 (m, 1H), 2.58 (q, J=7.6 Hz, 2H), 2.08 (s, 3H), 1.16 (t, J=7.6 Hz, 3H).

### Example 678C 2-chloro-5-ethylaniline

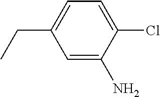

Aq. cc. HCl solution (7 mL, 12 M) was added slowly to the stirred solution of Example 678B (5.74 g, 29.04 mmol, 1 eq) in 1,4-dioxane (50 mL) and then heated at 85° C. for 7 h. The mixture was allowed to cool to rt and concentrated in vacuo until the volume was reduced by half. The mixture was diluted with DCM and washed with water and 2 M aq. NaOH solution. The organic phase was dried (MgSO₄) and concentrated in vacuo to afford Example 678C as an orange oil (4.10 g, 26.35 mmol, 91%). LRMS calculated for C₈H₁₀ClN: 155; found: 156 (M+H). ¹H NMR (400 MHz, DMSO-d₆) δ ppm: 7.06 (d, J=8.1 Hz, 1H), 6.63 (d, J=2.1 Hz, 1H), 6.39 (dd, J=8.1 Hz, 2.1 Hz, 1H), 5.21 (s, 2H), 2.45 (q, J=7.6 Hz, 2H), 1.12 (t, J=7.6 Hz, 3H).

### Example 678D 5-[(2-chloro-5-ethylanilino)methylidene]-2,2-dimethyl-1,3-dioxane-4,6-dione

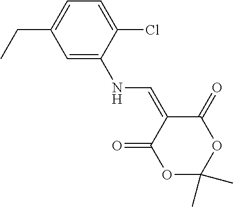 [SMILES: CCc1ccc(Cl)c(NC=C2C(=O)OC(C)(C)OC2=O)c1]

5-(Methoxymethylene)-2,2-dimethyl-1,3-dioxane-4,6-dione (5.39 g, 28.98 mmol, 1.1 eq) was added to a solution of Example 678C (4.1 g, 26.35 mmol, 1 eq) in EtOH (100 mL) and the suspension was stirred at rt for 3 h. The precipitate was collected by filtration, washed with EtOH and dried in vacuo to afford Example 678D as a cream powder (7.63 g, 24.63 mmol, 94%). ¹H NMR (400 MHz, DMSO-d₆) δ ppm: 11.61 (d, J=14.0 Hz, 1H), 8.85 (d, J=14.0 Hz, 1H), 7.84 (d, J=2.0 Hz, 1H), 7.51 (d, J=8.2 Hz, 1H), 7.15 (dd, J=8.2, 2.0 Hz, 1H), 2.66 (q, J=7.6 Hz, 2H), 1.70 (s, 6H), 1.22 (t, J=7.6 Hz, 3H).

### Example 678E 8-chloro-5-ethylquinolin-4-ol

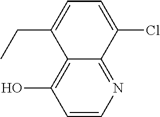

Example 678D (7.67 g, 24.76 mmol, 1 eq) was suspended in Ph₂O (70 mL), sparged with N₂ and then placed in a heating block, pre-heated to 260° C. and stirred rapidly at 250-260° C. for 45 min. The mixture was allowed to cool to rt, diluted with heptane (100 mL) and the mixture was stirred for 10 min. The precipitate was collected by filtration, washed with further heptane and the solids were dried in vacuo to afford Example 678E as a brown powder (4.3 g, 20.71 mmol, 84%). LRMS calculated for C₁₁H₁₀ClNO: 207; found: 208 (M+H). ¹H NMR (400 MHz, DMSO-d₆) δ ppm: 11.12-11.01 (br m, 1H), 7.75 (dd, J=7.5, 5.9 Hz, 1H), 7.70 (d, J=8.0 Hz, 1H), 7.05 (d, J=8.0 Hz, 1H), 6.07 (dd, J=7.5, 1.0 Hz, 1H), 3.28 (q, J=7.3 Hz, 2H), 1.15 (t, J=7.3 Hz, 3H).

### Example 678F 5-ethyl-5,6,7,8-tetrahydroquinolin-4-ol

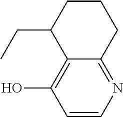

To a solution of Example 678E (4.3 g, 20.71 mmol, 1 eq) in TFA (100 mL, 1341.83 mmol, 64.8 eq) under N₂ was added PtO₂ (235 mg, 1.04 mmol, 0.05 eq). The mixture was evacuated and backfilled with N₂ (×3), then evacuated and backfilled with H₂ and heated at 57° C., with stirring, under an atmosphere of H₂ until no further conversion was observed. The mixture was allowed to cool to rt, filtered through celite, washed with a minimal mixture of DCM and MeOH. The solvents were removed in vacuo and the residue was partitioned between DCM/IPA (3:1) and sat. aq. NaHCO₃ solution. The phases were separated, and the aq. phase was extracted with DCM/IPA (3:1). The aq. phase was filtered, and the filter cake was washed with Et₂O and dried in vacuo to afford a racemate, Example 678F, isolated as a grey powder (1.1 g, 6.21 mmol, 30%). The organic phase was washed with brine, dried (MgSO₄), concentrated and dried in vacuo to afford a racemate, Example 678F, isolated as a brown foam (2.71 g, 13.76 mmol, 66%). LRMS calculated for C_{II}H₁₅NO: 177; found: 178 (M+H). ¹H NMR (400 MHz, DMSO-d₆) δ ppm: 11.62 (br s, 1H), 7.58 (d, J=7.1 Hz, 1H), 6.10 (d, J=7.1 Hz, 1H), 2.65-2.58 (m, 1H), 2.58-2.52 (m, 2H), 1.84-1.75 (m, 1H), 1.75-1.62 (m, 3H), 1.52-1.38 (m, 1H), 1.21-1.08 (m, 1H), 0.90 (t, J=7.4 Hz, 3H).

### Example 678G 4-chloro-5-ethyl-5,6,7,8-tetrahydroquinoline

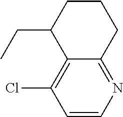

Example 678F (1.38 g, 7.79 mmol, 1 eq) was added portionwise to POCl₃ (7.26 mL, 77.86 mmol, 10 eq) with stirring at rt and then the mixture was heated at 100° C. for 10 h. The mixture was allowed to cool to rt and quenched by adding dropwise to rapidly stirred ice-water and stirred for 15 min. The mixture was basified with aq. NH₃ solution, extracted with DCM. Then the organic phase was dried (MgSO₄) and concentrated in vacuo. Purification by automated flash chromatography (Combiflash Rf, Silica 24 g RediSep™ cartridge) eluting with a gradient of 0-40% EtOAc in heptane afforded a racemate, Example 678G, isolated as a yellow oil (1.03 g, 5.26 mmol, 68%). LRMS calculated for C_{II}H₁₄ClN: 195; found: 196 (M+H). ¹H NMR (400 MHz, DMSO-d₆) δ ppm: 8.27 (d, J=5.2 Hz, 1H), 7.34-7.30 (m, 1H), 2.95-2.85 (m, 2H), 2.83-2.72 (m, 1H), 1.99-1.74 (m, 3H), 1.67-1.54 (m, 2H), 1.47-1.33 (m, 1H), 0.99 (t, J=7.4 Hz, 3H).

### Example 678H rac-(5R,8R)-4-chloro-5-ethyl-5,6,7,8-tetrahydroquinolin-8-ol

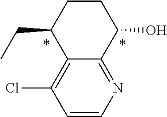

and

### Example 6781 rac-(5R,8S)-4-chloro-5-ethyl-5,6,7,8-tetrahydroquinolin-8-ol

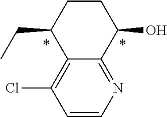

mCPBA (70% purity; 3.57 g, 14.47 mmol, 1.2 eq) was added portionwise to a solution of Example 678G (2.36 g, 12.06 mmol, 1 eq) in DCM (30 mL), cooled to 0° C. The mixture was slowly allowed to warm to rt and stirred for 2 h. 2 M aq. NaOH solution (30 mL) was added and the mixture was stirred for 10 min. The organic phase was separated, and the aq. phase was extracted with DCM. The combined organic extracts were washed with sat. aq. NaHCO₃ solution, dried (MgSO₄) and concentrated in vacuo to afford an intermediate that was dissolved in DCM (30 mL) and cooled to 0° C. under N₂. TFAA (5.03 mL, 36.18 mmol, 3 eq) was added dropwise and the mixture was allowed to slowly warm to rt and stirred for 18 h to afford a mixture of cis and trans isomers. 2 M aq. NaOH solution was added, and the mixture was stirred for 1 h. The phases were separated, and the aq. phase was extracted with DCM. The combined organic extracts were dried (MgSO₄) and concentrated in vacuo. Purification by automated flash chromatography (Combiflash Rf, Silica 40 g RediSep™ cartridge) eluting with a gradient of 0-50% EtOAc in heptane afforded the cis isomer, Example 6781, eluting firstly, isolated as a cream solid (1.09 g, 5.15 mmol, 43%). LRMS calculated for C_{II}H₁₄ClNO: 211; found: 212 (M+H). ¹H NMR (400 MHz, DMSO-d₆) δ ppm: 8.38 (d, J=5.2 Hz, 1H), 7.42 (dd, J=5.2, 0.7 Hz, 1H), 5.25 (d, J=3.7 Hz, 1H), 4.57-4.50 (m, 1H), 2.89-2.81 (m, 1H), 2.05-1.71 (m, 4H), 1.71-1.58 (m, 1H), 1.54-1.40 (m, 1H), 1.00 (t, J=7.3 Hz, 3H).

The trans isomer eluted later, Example 678H, isolated as a white powder (311 mg, 1.47 mmol, 12%). LRMS calculated for C₁₁H₁₄ClNO: 211; found: 212 (M+H). ¹H NMR (400 MHz, DMSO-d₆) δ ppm: 8.39 (d, J=5.2 Hz, 1H), 7.45 (d, J=5.2 Hz, 1H), 4.57-4.53 (m, 1H), 2.91-2.82 (m, 1H), 2.07-1.93 (m, 2H), 1.84-1.70 (m, 2H), 1.61-1.49 (m, 1H), 1.44-1.30 (m, 1H), 1.00 (t, J=7.3 Hz, 3H).

### Example 678J rac-(5R,8R)-4-chloro-5-ethyl-8-{[tri(propan-2-yl)silyl]oxy}-5,6,7,8-tetrahydroquinoline

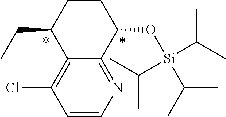 [SMILES: CC[C@H]1CC[C@H](O[Si](C(C)C)(C(C)C)C(C)C)c2nccc(Cl)c21]

TIPSOTf (1.92 mL, 7.2 mmol, 2 eq) was added dropwise to a solution of Example 678H (762 mg, 3.6 mmol, 1 eq) and DIPEA (1.79 mL, 10.8 mmol, 3 eq) in DCM (30 mL), cooled to 0° C. under N₂. The mixture was stirred at rt for 18 h. The mixture was partitioned with sat. aq. NH₄Cl solution and the phases were separated. The aq. phase was extracted with DCM, and the combined organic extracts were dried (MgSO₄) and concentrated in vacuo. Purification by automated flash chromatography (Combiflash Rf, Silica 24 g RediSep™ cartridge) eluting with a gradient of 0-5% EtOAc in heptane afforded a racemate, Example 678J, isolated as a colourless oil (1.14 g, 3.08 mmol, 86%). LRMS calculated for C₂₀H₃₄ClNOSi: 367; found: 368 (M+H). ¹H NMR (400 MHz, DMSO-d₆) δ ppm: 8.36 (d, J=5.2 Hz, 1H), 7.45 (d, J=5.2 Hz, 1H), 4.87-4.83 (m, 1H), 2.90-2.83 (m, 1H), 2.15-2.01 (m, 2H), 1.94-1.74 (m, 2H), 1.60-1.47 (m, 1H), 1.43-1.30 (m, 1H), 1.18-0.94 (m, 24H).

### Example 678K rac-(5R,8R)-5-ethyl-8-{[tri(propan-2-yl)silyl]oxy}-5,6,7,8-tetrahydroquinolin-4-ol

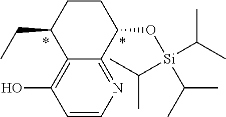 [SMILES: CC[C@H]1CC[C@H](O[Si](C(C)C)(C(C)C)C(C)C)c2nccc(O)c21]

Example 678J (1.14 g, 3.1 mmol, 1 eq) and K₂CO₃ (1.28 g, 9.29 mmol, 3 eq) were combined in DMF (19 mL) and water (1.9 mL) and the mixture was sparged with N₂. Herrmann's catalyst (58 mg, 61.95 μmol, 0.02 eq) and tBuXPhos (105 mg, 0.25 mmol, 0.08 eq) were added, the mixture was sparged with N₂ and then heated at 95° C. for 3 h under an atmosphere of N₂. The mixture was allowed to cool to rt, filtered through celite, washed with DCM/IPA (3:1) and the solvents were removed in vacuo. Purification by automated flash chromatography (Combiflash Rf, Silica 24 g RediSep™ cartridge) eluting with a gradient of 0-10% MeOH in DCM afforded a racemate, Example 678K, isolated as a yellow powder (968 mg, 2.77 mmol, 89%). LRMS calculated for C₂₀H₃₅NO₂Si: 349; found: 350 (M+H). ¹H NMR (400 MHz, DMSO-d₆) δ ppm: 10.65-10.54/10.45-10.37 (m, 1H), 8.10-8.02/7.59-7.50 (m, 1H), 6.72-6.64/6.02-5.94 (m, 1H), 4.77-4.67/4.67-4.57 (m, 1H), 2.78-2.68/2.64-2.52 (m, 1H), 2.09-0.83 (m, 30H).

### Example 678 (1r,2′S,4S)-4-(3-chloroanilino)-2′-[(2R)-3-{[(5S,8S)-5-ethyl-8-hydroxy-5,6,7,8-tetrahydroquinolin-4-yl]oxy}-2-methylpropyl]-2′,3′-dihydrospiro[cyclohexane-1,1′-indene]-4-carboxylic acid

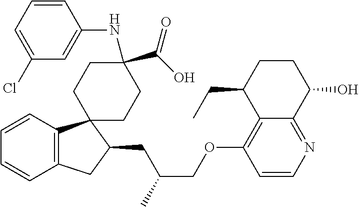 [SMILES: CC[C@H]1CC[C@H](O)c2nccc(OC[C@H](C)C[C@H]3Cc4ccccc4[C@]34CC[C@@](Nc3cccc(Cl)c3)(C(=O)O)CC4)c21]

and

### Example 679 (1r,2′S,4S)-4-(3-chloroanilino)-2′-[(2R)-3-{[(5R,8R)-5-ethyl-8-hydroxy-5,6,7,8-tetrahydroquinolin-4-yl]oxy}-2-methylpropyl]-2′,3′-dihydrospiro[cyclohexane-1,1′-indene]-4-carboxylic acid

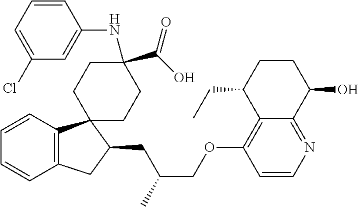 [SMILES: CC[C@@H]1CC[C@@H](O)c2nccc(OC[C@H](C)C[C@H]3Cc4ccccc4[C@]34CC[C@@](Nc3cccc(Cl)c3)(C(=O)O)CC4)c21]

Using General procedure 30a and Preparation 10b1 as the appropriate indane and Example 678K as the appropriate alcohol a mixture of diastereoisomers was obtained. TIPS was cleaved according to General Procedure 29. Then the diastereoisomers were separated by chiral chromatography. Column: AD, 50×500 mm, 20 μm, Eluents: EtOH/heptane. The diastereoisomer eluting earlier was hydrolyzed according to General procedure 33a to obtain Example 678. ¹H NMR (500 MHz, DMSO-d₆) δ ppm: 12.68 (br s, 1H), 8.25 (d, 1H), 7.41-7.10 (m, 4H), 7.04 (t, 1H), 6.86 (d, 1H), 6.62 (t, 1H), 6.53 (dm, 1H), 6.52 (dm, 1H), 6.19 (br s, 1H), 4.91 (d, 1H), 4.43 (m, 1H), 3.95/3.85 (dd+dd, 2H), 3.01/2.54 (dd+dd, 2H), 2.70 (m, 1H), 2.50-1.35 (m, 12H), 2.15 (m, 1H), 2.01 (m, 1H), 1.55/1.25 (m+m, 2H), 1.45/1.36 (m+m, 2H), 1.05 (d, 3H), 0.84 (t, 3H). HRMS calculated for C₃₆H₄₃N₂O₄Cl: 602.2911; found: 603.2983 (M+H).

The diastereoisomer eluting later was hydrolyzed according to General procedure 33a to obtain Example 679. ¹H NMR (500 MHz, DMSO-d₆) δ ppm: 12.68 (br s, 1H), 8.25 (d, 1H), 7.44-7.10 (m, 4H), 7.03 (t, 1H), 6.87 (d, 1H), 6.60 (t, 1H), 6.53 (m, 2H), 6.23 (br s, 1H), 4.91 (d, 1H), 4.43 (m, 1H), 3.91/3.88 (dd+dd, 2H), 3.01/2.54 (dd+dd, 2H), 2.72 (m, 1H), 2.50-1.28 (m, 12H), 2.14 (m, 1H), 2.01 (m, 1H), 1.53/1.36 (m+m, 2H), 1.50/1.19 (m+m, 2H), 1.04 (d, 3H), 0.75 (t, 3H). HRMS calculated for C₃₆H₄₃N₂O₄Cl: 602.2911; found: 603.2976 (M+H).

### Example 680

### Example 680A (8S)-8-fluoro-5,6,7,8-tetrahydroquinoline

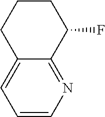 [SMILES: F[C@H]1CCCc2cccnc21]

To a solution of (8R)-5,6,7,8-tetrahydroquinolin-8-ol (500 mg, 3.35 mmol, 1 eq) in DCM (15 mL), cooled to 0° C., was added DAST (0.89 mL, 6.7 mmol, 2 eq) dropwise. The mixture was slowly warmed to rt and stirred for 72 h. The mixture was cooled to 0° C. and quenched by the dropwise addition of sat. aq. NH₄Cl solution, then basified to pH 11 with 2 M aq. NaOH solution and extracted with DCM. The organic phase was dried (MgSO₄) and concentrated in vacuo. Purification by automated flash chromatography (CombiFlash Rf, Silica 12 g RediSep™ cartridge) eluting with a gradient of 0-40% EtOAc in heptane afforded Example 680A, isolated as a yellow oil (408 mg, 2.7 mmol, 81%). LRMS calculated for C₉H₁₀FN: 151; found: 152 (M+H). ¹H NMR (400 MHz, DMSO-d₆) δ ppm: 8.50-8.45 (m, 1H), 7.66-7.60 (m, 1H), 7.34 (ddd, J=7.8, 4.7, 2.1 Hz, 1H), 5.58-5.41 (m, 1H), 2.90-2.66 (m, 2H), 2.32-2.19 (m, 1H), 2.08-1.88 (m, 1H), 1.85-1.76 (m, 2H).

### Example 680B (8S)-8-fluoro-1-oxo-5,6,7,8-tetrahydro-1λ⁵-quinoline

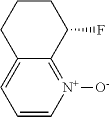 [SMILES: [O-][n+]1cccc2c1[C@@H](F)CCC2]

To a solution of Example 680A (408 mg, 2.7 mmol, 1 eq) in DCM (10 mL), cooled to 0° C., was added mCPBA (77% purity, 726 mg, 3.24 mmol, 1.2 eq) portionwise and the mixture was warmed slowly to rt and stirred for 4 h. The mixture was cooled to 0° C., sat. aq. NaHCO₃ solution was added slowly, and the mixture was stirred for 30 min. The mixture was partitioned between DCM and water, and the organic phase was washed with brine, dried (MgSO₄) and concentrated in vacuo. Purification by automated flash chromatography (CombiFlash Rf, Silica 4 g RediSep™ cartridge) eluting with a gradient of 0-9% MeOH in DCM afforded Example 680B as a yellow oil (381 mg, 2.28 mmol, 85%). LRMS calculated for C₉H₁₀FNO: 167; found: 168 (M+H). ¹H NMR (400 MHz, DMSO-d₆) δ ppm: 8.20 (d, J=6.4 Hz, 1H), 7.39 (ddd, J=8.2, 6.4, 2.0 Hz, 1H), 7.22-7.16 (m, 1H), 6.03-5.87 (m, 1H), 2.93-2.83 (m, 1H), 2.77-2.63 (m, 1H), 2.33-2.21 (m, 1H), 1.91-1.63 (m, 3H).

### Example 680C (8S)-4-chloro-8-fluoro-5,6,7,8-tetrahydroquinoline

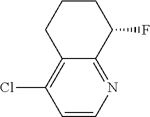 [SMILES: F[C@H]1CCCc2c(Cl)ccnc21]

A solution of Example 680B (381 mg, 2.28 mmol, 1 eq) in POCl₃ (1.7 mL, 18.2 mmol, 8 eq) was heated at 70° C. for 18 h. The mixture was cooled to rt and added dropwise to rapidly stirring ice-water and then basified with aq. NH₃ solution. The mixture was extracted with DCM and the organic phase was washed with brine, dried (MgSO₄) and concentrated in vacuo. Purification by automated flash chromatography (CombiFlash Rf, Silica 12 g RediSep™ cartridge) eluting with a gradient of 0-32% EtOAc in heptane afforded Example 680C as a colourless solid (174 mg, 0.94 mmol, 41%). LRMS calculated for C₉H₉ClFN: 185; found: 186 (M+H). ¹H NMR (400 MHz, DMSO-d₆) δ ppm: 8.50-8.44 (m, 1H), 7.59 (dd, J=5.2, 1.9 Hz, 1H), 5.61-5.44 (m, 1H), 2.98-2.88 (m, 1H), 2.71-2.58 (m, 1H), 2.31-2.18 (m, 1H), 2.08-1.76 (m, 3H).

### Example 680D methyl (1r,2′S,4S)-4-(3-chloroanilino)-2′-[(2R)-3-{[(8S)-8-fluoro-5,6,7,8-tetrahydroquinolin-4-yl]oxy}-2-methylpropyl]-2′,3′-dihydrospiro[cyclohexane-1,1′-indene]-4-carboxylate

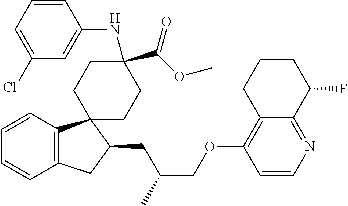 [SMILES: COC(=O)[C@]1(Nc2cccc(Cl)c2)CC[C@@]2(CC1)c1ccccc1C[C@@H]2C[C@@H](C)COc1ccnc2c1CCC[C@@H]2F]

Using General procedure 31a and Preparation 10b1 (150 mg, 0.34 mmol, 1 eq) as the appropriate alcohol and Example 680C (75.6 mg, 0.41 mmol, 1.2 eq) as the appropriate aryl chloride, Example 680D was isolated as a cream powder (119 mg, 0.2 mmol, 59%). LRMS calculated for C₃₅H₄₀ClFN₂O₃: 590; found: 591 (M+H). ¹H NMR (400 MHz, DMSO-d₆) δ ppm: 8.34 (d, J=5.6 Hz, 1H), 7.43-7.36 (m, 1H), 7.24-7.12 (m, 3H), 7.06 (t, J=8.1 Hz, 1H), 7.01-6.96 (m, 1H), 6.62-6.54 (m, 2H), 6.48-6.43 (m, 1H), 6.34 (s, 1H), 5.52-5.35 (m, 1H), 3.98-3.89 (m, 2H), 3.65 (s, 3H), 3.00 (dd, J=15.6, 7.0 Hz, 1H), 2.82-2.69 (m, 1H), 2.59-2.32 (m, 3H), 2.27-1.66 (m, 11H), 1.54-1.30 (m, 4H), 1.05 (d, J=6.6 Hz, 3H).

### Example 680 (1r,2′S,4S)-4-(3-chloroanilino)-2′-[(2R)-3-{[(8S)-8-fluoro-5,6,7,8-tetrahydroquinolin-4-yl]oxy}-2-methylpropyl]-2′,3′-dihydrospiro[cyclohexane-1,1′-indene]-4-carboxylic acid

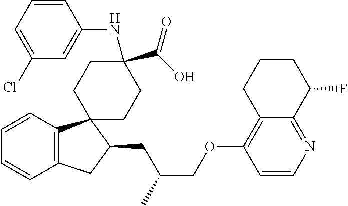 [SMILES: C[C@@H](COc1ccnc2c1CCC[C@@H]2F)C[C@H]1Cc2ccccc2[C@]12CC[C@@](Nc1cccc(Cl)c1)(C(=O)O)CC2]

Using General procedure 33b and Example 680D (119 mg, 0.2 mmol, 1 eq) as the appropriate ester, Example 680 was isolated as an off-white powder (2.4 mg, 4.16 μmol, 2%). LRMS calculated for C₃₄H₃₈ClFN₂O₃: 576; found: 577 (M+H). ¹H NMR (400 MHz, DMSO-d₆) δ ppm: 8.34 (d, J=5.7 Hz, 1H), 7.42-7.35 (m, 1H), 7.24-7.11 (m, 3H), 7.04 (t, J=8.1 Hz, 1H), 7.01-6.96 (m, 1H), 6.65-6.60 (m, 1H), 6.58-6.51 (m, 2H), 5.52-5.35 (m, 1H), 3.97-3.89 (m, 2H), 3.00 (dd, J=15.6, 7.0 Hz, 1H), 2.82-2.69 (m, 1H), 2.60-2.33 (m, 3H), 2.26-1.61 (m, 11H), 1.55-1.31 (m, 4H), 1.05 (d, J=6.6 Hz, 3H).

### Example 681

### Example 681A (8R)-8-fluoro-5,6,7,8-tetrahydroquinoline

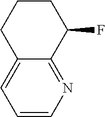 [SMILES: F[C@@H]1CCCc2cccnc21]

To a solution of (8S)-5,6,7,8-tetrahydroquinolin-8-ol (500 mg, 3.35 mmol, 1 eq) in DCM (15 mL), cooled to 0° C., was added DAST (0.89 mL, 6.7 mmol, 2 eq) dropwise. The mixture was slowly warmed to rt over 18 h. The mixture was cooled to 0° C., quenched by the dropwise addition of sat. aq. NH₄Cl solution and then basified to pH 11 with 2 M aq. NaOH solution.

The mixture was extracted with DCM and the organic phase was dried (MgSO₄) and concentrated in vacuo. Purification by automated flash chromatography (CombiFlash Rf, Silica 12 g RediSep™ cartridge) eluting with a gradient of 0-36% EtOAc in heptane afforded Example 681A as a yellow oil (373 mg, 2.47 mmol, 74%). LRMS calculated for C₉H₁₀FN: 151; found: 152 (M+H). ¹H NMR (400 MHz, DMSO-d₆) δ ppm: 8.50-8.45 (m, 1H), 7.65-7.60 (m, 1H), 7.34 (ddd, J=7.8, 4.7, 2.1 Hz, 1H), 5.57-5.41 (m, 1H), 2.90-2.80 (m, 1H), 2.80-2.66 (m, 1H), 2.31-2.19 (m, 1H), 2.09-1.88 (m, 1H), 1.85-1.76 (m, 2H).

### Example 681B (8R)-8-fluoro-1-oxo-5,6,7,8-tetrahydro-1λ⁵-quinoline

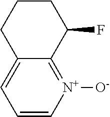 [SMILES: [O-][n+]1cccc2c1[C@H](F)CCC2]

To a solution of Example 681A (400 mg, 2.65 mmol, 1 eq) in DCM (10 mL), cooled to 0° C., was added mCPBA (77% purity, 711 mg, 3.18 mmol, 1.2 eq) portionwise and the mixture was stirred at 0° C. for 3 h, then at rt for 18 h. The mixture was cooled to 0° C., sat. aq. NaHCO₃ solution was added slowly, and the mixture was stirred for 30 min. The mixture was partitioned between DCM and water, and the organic phase was washed with brine, dried (MgSO₄) and concentrated in vacuo. Purification by automated flash chromatography (CombiFlash Rf, Silica 12 g RediSep™ cartridge) eluting with a gradient of 0-9% MeOH in DCM afforded Example 681B as a yellow oil. LRMS calculated for C₉H₁₀FNO: 167; found: 168 (M+H). ¹H NMR (400 MHz, DMSO-d₆) δ ppm: 8.20 (d, J=6.4 Hz, 1H), 7.39 (ddd, J=8.2, 6.4, 2.0 Hz, 1H), 7.22-7.17 (m, 1H), 6.03-5.87 (m, 1H), 2.93-2.83 (m, 1H), 2.77-2.65 (m, 1H), 2.35-2.21 (m, 1H), 1.91-1.67 (m, 3H).

### Example 681C (8R)-4-chloro-8-fluoro-5,6,7,8-tetrahydroquinoline

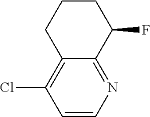 [SMILES: F[C@@H]1CCCc2c(Cl)ccnc21]

A solution of Example 681B (461 mg, 2.76 mmol, 1 eq) in POCl₃ (2.57 mL, 27.6 mmol, 10 eq) was heated at 70° C. for 18 h. The mixture was cooled to rt and added dropwise to rapidly stirring ice-water and stirred for 30 min. The mixture was basified with aq. NH₃ solution, extracted with DCM and the organic phase was washed with brine, dried (MgSO₄) and concentrated in vacuo. Purification by automated flash chromatography (CombiFlash Rf, Silica 24 g RediSep™ cartridge) eluting with a gradient of 0-36% EtOAc in heptane afforded Example 681C as a colourless solid (167 mg, 0.9 mmol, 33%). LRMS calculated for C₉H₉ClFN: 185; found: 186 (M+H). ¹H NMR (400 MHz, DMSO-d₆) δ ppm: 8.50-8.44 (m, 1H), 7.59 (dd, J=5.2, 1.9 Hz, 1H), 5.61-5.44 (m, 1H), 2.98-2.88 (m, 1H), 2.71-2.58 (m, 1H), 2.31-2.18 (m, 1H), 2.08-1.76 (m, 3H).

### Example 681D methyl (1r,2′S,4S)-4-(3-chloroanilino)-2′-[(2R)-3-{[(8R)-8-fluoro-5,6,7,8-tetrahydroquinolin-4-yl]oxy}-2-methylpropyl]-2′,3′-dihydrospiro[cyclohexane-1,1′-indene]-4-carboxylate

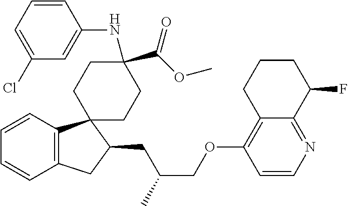 [SMILES: COC(=O)[C@]1(Nc2cccc(Cl)c2)CC[C@@]2(CC1)c1ccccc1C[C@@H]2C[C@@H](C)COc1ccnc2c1CCC[C@H]2F]

Using General procedure 31a and Preparation 10b1 (150 mg, 0.34 mmol, 1 eq) as the appropriate alcohol and Example 681C (75.6 mg, 0.41 mmol, 1.2 eq) as the appropriate aryl chloride, Example 681D was obtained as a cream powder (59 mg, 0.1 mmol, 29%). LRMS calculated for C₃₅H₄₀ClFN₂O₃: 590; found: 591 (M+H). ¹H NMR (400 MHz, DMSO-d₆) δ ppm: 8.34 (d, J=5.6 Hz, 1H), 7.43-7.36 (m, 1H), 7.23-7.12 (m, 3H), 7.06 (t, J=8.1 Hz, 1H), 7.01-6.96 (m, 1H), 6.62-6.54 (m, 2H), 6.48-6.43 (m, 1H), 6.34 (s, 1H), 5.51-5.35 (m, 1H), 3.98-3.89 (m, 2H), 3.66 (s, 3H), 3.00 (dd, J=15.6, 7.0 Hz, 1H), 2.83-2.73 (m, 1H), 2.59-2.31 (m, 3H), 2.27-1.67 (m, 11H), 1.53-1.32 (m, 4H), 1.05 (d, J=6.7 Hz, 3H).

### Example 681 (1r,2′S,4S)-4-(3-chloroanilino)-2′-[(2R)-3-{[(8R)-8-fluoro-5,6,7,8-tetrahydroquinolin-4-yl]oxy}-2-methylpropyl]-2′,3′-dihydrospiro[cyclohexane-1,1′-indene]-4-carboxylic acid

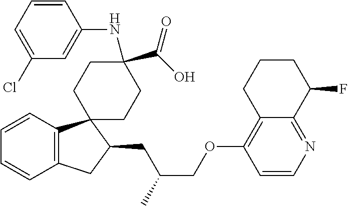 [SMILES: C[C@@H](COc1ccnc2c1CCC[C@H]2F)C[C@H]1Cc2ccccc2[C@]12CC[C@@](Nc1cccc(Cl)c1)(C(=O)O)CC2]

Using General procedure 33b and Example 681D (59 mg, 0.1 mmol, 1 eq) as the appropriate ester, Example 681 was obtained as an off-white powder (9.2 mg, 0.02 mmol, 16%). LRMS calculated for C₃₄H₃₈ClFN₂O₃: 576; found: 577 (M+H). ¹H NMR (400 MHz, DMSO-d₆) δ ppm: 8.34 (d, J=5.5 Hz, 1H), 7.42-7.35 (m, 1H), 7.24-7.12 (m, 3H), 7.05 (t, J=8.1 Hz, 1H), 7.01-6.96 (m, 1H), 6.62 (t, J=2.2 Hz, 1H), 6.57-6.52 (m, 2H), 5.51-5.35 (m, 1H), 3.98-3.89 (m, 2H), 3.00 (dd, J=15.6, 7.0 Hz, 1H), 2.83-2.73 (m, 1H), 2.59-2.30 (m, 3H), 2.26-1.65 (m, 11H), 1.54-1.30 (m, 4H), 1.05 (d, J=6.6 Hz, 3H).

### Example 682 and Example 683 and Example 684

### Example 682A 8-(difluoromethyl)quinoline

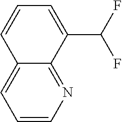 [SMILES: FC(F)c1cccc2cccnc12]

DAST (5.55 mL, 42 mmol, 2.2 eq) was added dropwise to a solution of 8-quinolinecarbaldehyde (3 g, 19.09 mmol, 1 eq) in anhydrous DCM (50 mL), cooled to 0° C. under N₂ and then allowed to warm to rt and stirred for 3 days under N₂. The mixture was diluted with DCM, cooled to 0° C. and slowly quenched by the careful addition of 2 M aq. NaOH solution. The phases were separated, and the aq. phase was extracted with DCM. The combined organic extracts were dried (MgsO₄) and concentrated in vacuo. Purification by automated flash chromatography (Combiflash Rf, Silica 40 g RediSep™ cartridge) eluting with a gradient of 0-32% EtOAc (3% TEA) in heptane afforded Example 682A as a yellow oil (1.99 g, 11.09 mmol, 58%). LRMS calculated for C₁₀H₇F₂N: 179; found: 180 (M+H). ¹H NMR (400 MHz, DMSO-d₆) δ ppm: 9.02 (dd, J=4.2, 1.8 Hz, 1H), 8.51 (dd, J=8.3, 1.8 Hz, 1H), 8.24-8.18 (m, 1H), 8.09-8.04 (m 1H), 7.90 (t, J=55.3 Hz, 1H), 7.79-7.74 (m, 1H), 7.68 (dd, J=8.3, 4.2 Hz, 1H).

### Example 682B 8-(difluoromethyl)-5,6,7,8-tetrahydroquinoline

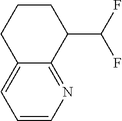 [SMILES: FC(F)C1CCCc2cccnc21]

PtO₂ (250 mg, 0.1 eq) was added to a solution of Example 682A (1.98 g, 11.05 mmol) in TFA (35 mL, 469.64 mmol, 65.74 eq) under a N₂ atmosphere. The mixture was evacuated and flushed with H₂ and then heated at 52° C. with stirring for 7 h under an atmosphere of H₂. The mixture was allowed to cool to rt and filtered through celite, washed with minimal MeOH. The solvents were removed in vacuo, and the residue was partitioned between DCM and 2 M aq. NaOH solution. The organic phase was dried (MgSO₄) and concentrated in vacuo. Purification by automated flash chromatography (Combiflash Rf, Silica 24 g RediSep™ cartridge) eluting with a gradient of 0-40% EtOAc (3% TEA) in heptane afforded a racemate, Example 682B, isolated as a yellow oil (831 mg, 4.54 mmol, 41%). LRMS calculated for C₁₀H₁₁F₂N: 183; found: 184 (M+H). ¹H NMR (400 MHz, DMSO-d₆) δ ppm: 8.42-8.37 (m, 1H), 7.58-7.53 (m, 1H), 7.22 (ddd, J=7.7, 4.7, 0.8 Hz, 1H), 6.72 (td, J=56.7, 2.3 Hz, 1H), 3.52-3.36 (m, 1H), 2.79-2.72 (m, 2H), 2.09-1.63 (m, 4H).

### Example 682C 8-(difluoromethyl)-1-oxo-5,6,7,8-tetrahydro-1λ⁵-quinoline

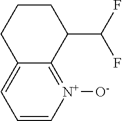 [SMILES: [O-][n+]1cccc2c1C(C(F)F)CCC2]

mCPBA (70% purity; 1.34 g, 5.44 mmol, 1.2 eq) was added portionwise to a solution of Example 682B (831 mg, 4.54 mmol, 1 eq) in DCM (15 mL), cooled to 0° C., and the mixture was stirred for 3 h. 2 M aq. NaOH solution was added and the mixture was stirred for 10 min. The layers were separated, and the aq. phase was extracted with DCM/IPA (3:1). The combined organic extracts were dried (MgSO₄) and concentrated in vacuo to afford a racemate, Example 682C, isolated as an off-white solid (809 mg, 4.06 mmol, 90%). LRMS calculated for C₁₀H₁₁F₂NO: 199; found: 200 (M+H). ¹H NMR (400 MHz, DMSO-d₆) δ ppm: 8.24-8.19 (m, 1H), 7.37-7.30 (m, 1H), 7.25-7.20 (m, 1H), 6.93-6.61 (m, 1H), 3.90-3.74 (m, 1H), 2.84-2.67 (m, 2H), 2.27-2.15 (m, 1H), 1.89-1.63 (m, 3H).

### Example 682D 4-chloro-8-(difluoromethyl)-5,6,7,8-tetrahydroquinoline

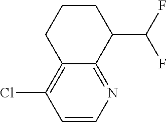 [SMILES: FC(F)C1CCCc2c(Cl)ccnc21]

Example 682C (800 mg, 4.02 mmol, 1 eq) was cooled to 0° C. and POCl₃ (3.74 mL, 40.16 mmol, 10 eq) was added slowly. The suspension was stirred for 5 min at 0° C. and then slowly heated to 80° C. and stirred for 3 h. A mixture of regioisomers was observed. The mixture was allowed to cool to rt, and then quenched by the dropwise addition to rapidly stirred ice-water. The solution was basified to pH 11 with aq. NH₃ solution and extracted with DCM. The organic phase was dried (MgSO₄) and concentrated in vacuo. Purification by automated flash chromatography (Combiflash Rf, Silica 24 g RediSep™ cartridge) eluting with a gradient of 0-27% EtOAc (3% TEA) in heptane afforded a racemate, Example 682D, isolated as a white solid (531 mg, 2.44 mmol, 61%). LRMS calculated for C₁₀H₁₀ClF₂N: 217; found: 218 (M+H). ¹H NMR (400 MHz, DMSO-d₆) δ ppm: 8.41-8.37 (m, 1H), 7.47 (dd, J=5.2, 0.8 Hz, 1H), 6.73 (td, J=56.6, 2.4 Hz, 1H), 3.58-3.42 (m, 1H), 2.87-2.77 (m, 1H), 2.76-2.66 (m, 1H), 2.09-1.93 (m, 2H), 1.92-1.67 (m, 2H).

### Example 682 (1r,2′S,4S)-4-(3-chloroanilino)-2′-[(2R)-3-{[8-(difluoromethyl)-5,6,7,8-tetrahydroquinolin-4-yl]oxy}-2-methylpropyl]-2′,3′-dihydrospiro[cyclohexane-1,1′-indene]-4-carboxylic acid, diastereoisomer 1

and

### Example 683 (1r,2′S,4S)-4-(3-chloroanilino)-2′-[(2R)-3-{[8-(difluoromethyl)-5,6,7,8-tetrahydroquinolin-4-yl]oxy}-2-methylpropyl]-2′,3′-dihydrospiro[cyclohexane-1,1′-indene]-4-carboxylic acid, diastereoisomer 2

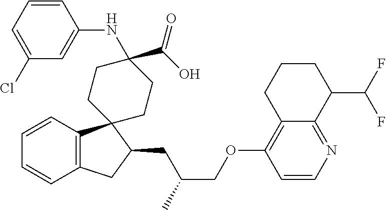 [SMILES: C[C@@H](COc1ccnc2c1CCCC2C(F)F)C[C@H]1Cc2ccccc2[C@]12CC[C@@](Nc1cccc(Cl)c1)(C(=O)O)CC2]

and

### Example 684 (1r,2′S,4S)-4-(3-chloroanilino)-2′-[(2R)-3-{[(8E)-8-(fluoromethylidene)-5,6,7,8-tetrahydroquinolin-4-yl]oxy}-2-methylpropyl]-2′,3′-dihydrospiro[cyclohexane-1,1′-indene]-4-carboxylic acid

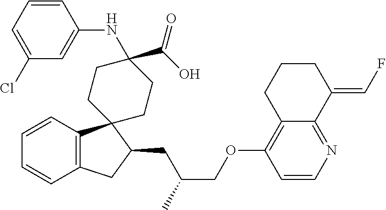 [SMILES: C[C@@H](COc1ccnc2c1CCC/C2=C\F)C[C@H]1Cc2ccccc2[C@]12CC[C@@](Nc1cccc(Cl)c1)(C(=O)O)CC2]

Using General procedure 31a and Preparation 10b1 as the appropriate indane and Example 682D as the appropriate aryl chloride, a mixture of diastereoisomers was obtained. The diastereoisomers were separated by chiral chromatography. Column: AD, 50×500 mm, 20 μm, Eluents: EtOH/heptane. The diastereoisomer eluting earlier was hydrolyzed according to General Procedure 33a to obtain Example 682. HRMS calculated for C₃₅H₃₉N₂O₃F₂Cl: 608.2617; found: 609.2697 (M+H).

The diastereoisomer eluting later was hydrolyzed according to General procedure 33a to obtain Example 683. HRMS calculated for C₃₅H₃₉N₂O₃F₂Cl: 608.2617; found: 609.2688 (M+H).

During the hydrolysis of Example 682 HF elimination was partly occurred and the by-product was isolated as Example 684. HRMS calculated for C₃₅H₃₈N₂O₃FCl: 588.2555; found: 589.2628 (M+H).

### Example 685 and Example 686

### Example 685A 4-chloro-5-fluoro-5,6,7,8-tetrahydroquinoline

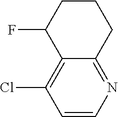 [SMILES: FC1CCCc2nccc(Cl)c21]

DAST (0.51 mL, 3.86 mmol, 2 eq) was added dropwise to a suspension of Example 671C (354 mg, 1.93 mmol, 1 eq) in DCM (10 mL), cooled to 0° C. under N₂ and then stirred for 3 h at rt. The mixture was cooled to 0° C. and sat. aq. NH₄Cl (3 mL) was added dropwise followed by the slow addition of 2 M aq. NaOH solution (15 mL). The mixture was diluted with DCM and the organics collected (PTFE phase separator) and concentrated in vacuo. Purification by automated flash chromatography (Combiflash Rf, Silica 12 g RediSep™ cartridge) eluting with a gradient of 0-35% EtOAc (3% TEA) in heptane afforded a racemate, Example 685A, isolated as a colourless oil (269 mg, 1.45 mmol, 75%). LRMS calculated for C₉H₉ClFN: 185; found: 186 (M+H). ¹H NMR (400 MHz, DMSO-d₆) δ ppm: 8.49 (dd, J=5.2, 2.2 Hz, 1H), 7.51-7.47 (m, 1H), 5.97-5.81 (m, 1H), 3.02-2.92 (m, 1H), 2.89-2.76 (m, 1H), 2.35-2.20 (m, 1H), 2.00-1.78 (m, 3H).

### Example 685 (1r,2′S,4S)-4-(3-chloroanilino)-2′-{(2R)-3-[(5-fluoro-5,6,7,8-tetrahydroquinolin-4-yl)oxy]-2-methylpropyl}-2′,3′-dihydrospiro[cyclohexane-1,1′-indene]-4-carboxylic acid, diastereoisomer 1

and

### Example 686 (1r,2′S,4S)-4-(3-chloroanilino)-2′-{(2R)-3-[(5-fluoro-5,6,7,8-tetrahydroquinolin-4-yl)oxy]-2-methylpropyl}-2′,3′-dihydrospiro[cyclohexane-1,1′-indene]-4-carboxylic acid, diastereoisomer 2

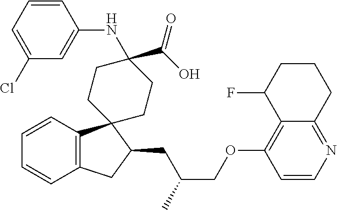 [SMILES: C[C@@H](COc1ccnc2c1C(F)CCC2)C[C@H]1Cc2ccccc2[C@]12CC[C@@](Nc1cccc(Cl)c1)(C(=O)O)CC2]

Using General procedure 31a and Preparation 10b1 as the appropriate indane and Example 685A as the appropriate aryl chloride, a mixture of diastereoisomers was obtained. The diastereoisomers were separated by chiral chromatography. Column: IC, 50×500 mm, 20 μm, Eluents: EtOH/heptane. The diastereoisomer eluting earlier was hydrolyzed according to General Procedure 33a to obtain Example 685. ¹H NMR (500 MHz, DMSO-d₆) δ ppm: 12.65 (br s, 1H), 8.33 (dm, 1H), 7.37 (dm, 1H), 7.20 (dm, 1H), 7.16-7.13 (m, 2H), 7.05 (t, 1H), 6.91 (d, 1H), 6.62 (t, 1H), 6.55 (dd, 1H), 6.54 (dd, 1H), 6.21 (br s, 1H), 5.78/5.68 (m+m, 1H), 3.92 (m, 2H), 2.99/2.56 (dd+dd, 2H), 2.83/2.68 (m+m, 2H), 2.42-1.40 (m, 12H), 2.14 (m, 1H), 2.00 (m, 1H), 1.41/1.31 (m+m, 2H), 1.04 (d, 3H). HRMS calculated for C₃₄H₃₈N₂O₃FCl: 576.2555; found: 577.2628 (M+H).

The diastereoisomer eluting later was hydrolyzed according to General procedure 33a to obtain Example 686. ¹H NMR (500 MHz, DMSO-d₆) δ ppm: 12.83 (br s, 1H), 8.33 (d, 1H), 7.42-7.10 (m, 4H), 7.02 (t, 1H), 6.92 (d, 1H), 6.62 (t, 1H), 6.54 (dm, 1H), 6.51 (dm, 1H), 6.17 (br s, 1H), 5.50 (dm, 1H), 4.01/3.90 (dd+dd, 2H), 2.98/2.52 (dd+dd, 2H), 2.90-1.29 (m, 17H), 2.11 (m, 1H), 1.04 (d, 3H). HRMS calculated for C₃₄H₃₈N₂O₃FCl: 576.2555; found: 577.2624 (M+H).

### Example 687 and Example 688 and Example 689 and Example 690

### Example 687A rac-(5R,8S)-4-chloro-8-fluoro-5-methyl-5,6,7,8-tetrahydroquinoline

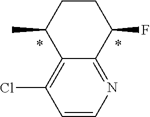

To a solution of Example 675A (232 mg, 1.17 mmol, 1 eq) in DCM (10 mL), cooled to 0° C., was added DAST (0.31 mL, 2.35 mmol, 2 eq). The mixture was slowly warmed to rt and stirred for 2 h. A mixture of diastereoisomers was obtained. The mixture was cooled to 0° C., quenched by the dropwise addition of sat. aq. NH₄Cl solution (1 mL), followed by the careful addition of 2 M aq. NaOH solution (10 mL). The mixture was extracted with DCM and the combined organic extracts were washed with brine, dried (PTFE phase separator) and concentrated in vacuo. The diastereoisomer were separated by automated flash chromatography (CombiFlash Rf, Silica Gold 4 g RediSep™ cartridge) eluting with a gradient of 0-30% EtOAc in heptane. The diastereoisomer eluting later was collected as Example 687A, isolated as a colourless oil (125 mg, 0.63 mmol, 53%). LRMS calculated for C₁₀H₁₁ClFN: 199; found: 200 (M+H). ¹H NMR (400 MHz, DMSO-d₆) δ ppm: 8.49-8.46 (m, 1H), 7.59 (dd, J=5.2, 2.1 Hz, 1H), 5.55-5.39 (m, 1H), 3.33-3.24 (m, 1H), 2.38-1.98 (m, 3H), 1.71-1.63 (m, 1H), 1.20 (d, J=7.0 Hz, 3H).

### Example 689A rac-(5R,8R)-4-chloro-8-fluoro-5-methyl-5,6,7,8-tetrahydroquinoline

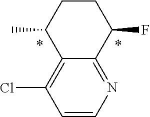

To a solution of Example 673E (220 mg, 1.11 mmol, 1 eq) in DCM (10 mL), cooled to 0° C., was added DAST (0.29 mL, 2.23 mmol, 2 eq). The mixture was slowly warmed to rt and stirred for 2 h. A mixture of diastereoisomers was obtained. The mixture was cooled to 0° C., quenched by the dropwise addition of sat. aq. NH₄Cl solution (1 mL) followed by the careful addition of 2 M aq. NaOH solution (10 mL). The mixture was extracted with DCM and the combined organic extracts were washed with brine, dried (MgSO₄) and concentrated in vacuo. The diastereoisomer were separated by automated flash chromatography (CombiFlash Rf, Silica 4 g RediSep™ cartridge) eluting with a gradient of 0-50% EtOAc in heptane. The diastereoisomer eluting earlier was collected as Example 689A, isolated as a colourless oil (150 mg, 0.75 mmol, 67%). LRMS calculated for C₁₀H₁₁ClFN: 199; found: 200 (M+H). ¹H NMR (400 MHz, DMSO-d₆) δ ppm: 8.45 (d, J=5.1 Hz, 1H), 7.55-7.51 (m, 1H), 5.59 (ddd, J=49.9, 8.6, 6.5 Hz, 1H), 3.25-3.15 (m, 1H), 2.27-2.06 (m, 2H), 2.06-1.95 (m, 1H), 1.79-1.69 (m, 1H), 1.30 (d, J=7.0 Hz, 3H).

### Example 687 (1r,2′S,4S)-4-(3-chloroanilino)-2′-[(2R)-3-{[(5R,8S)-8-fluoro-5-methyl-5,6,7,8-tetrahydroquinolin-4-yl]oxy}-2-methylpropyl]-2′,3′-dihydrospiro[cyclohexane-1,1′-indene]-4-carboxylic acid

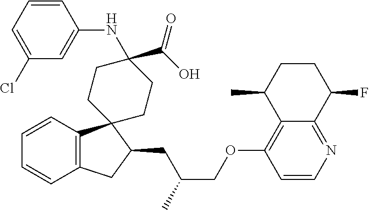 [SMILES: C[C@@H](COc1ccnc2c1[C@@H](C)CC[C@H]2F)C[C@H]1Cc2ccccc2[C@]12CC[C@@](Nc1cccc(Cl)c1)(C(=O)O)CC2]

### Example 688 (1r,2′S,4S)-4-(3-chloroanilino)-2′-[(2R)-3-{[(5S,8R)-8-fluoro-5-methyl-5,6,7,8-tetrahydroquinolin-4-yl]oxy}-2-methylpropyl]-2′,3′-dihydrospiro[cyclohexane-1,1′-indene]-4-carboxylic acid

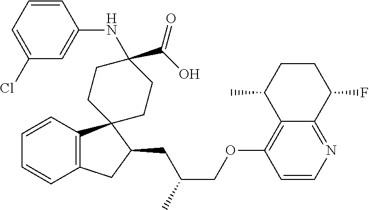 [SMILES: C[C@@H](COc1ccnc2c1[C@H](C)CC[C@@H]2F)C[C@H]1Cc2ccccc2[C@]12CC[C@@](Nc1cccc(Cl)c1)(C(=O)O)CC2]

Using General procedure 31a and Preparation 10b1 as the appropriate indane and Example 687A as the appropriate aryl chloride, a mixture of diastereoisomers was obtained. The diastereoisomers were separated by chiral chromatography. Column: AD, 50×500 mm, 20 μm, Eluents: nPrOH/heptane. The diastereoisomer eluting earlier was hydrolyzed according to General Procedure 33a to obtain Example 687. ¹H NMR (500 MHz, DMSO-d₆) δ ppm: 12.74 (br s, 1H), 8.31 (d, 1H), 7.38-7.10 (m, 4H), 7.00 (t, 1H), 6.95 (d, 1H), 6.64 (t, 1H), 6.54 (dm, 1H), 6.49 (dm, 1H), 6.10 (br s, 1H), 5.45 (dm, 1H), 3.97/3.85 (dd+dd, 2H), 3.00/2.54 (dd+dd, 2H), 2.96 (m, 1H), 2.50-1.30 (m, 15H), 2.16 (m, 1H), 1.23 (d, 3H), 1.05 (d, 3H). HRMS calculated for C₃₅H₄₀N₂O₃FCl: 590.2712; found: 591.2781 (M+H).

The diastereoisomer eluting later was hydrolyzed according to General procedure 33a to obtain Example 688. ¹H NMR (500 MHz, DMSO-d₆) δ ppm: 12.69 (br s, 1H), 8.31 (d, 1H), 7.40-7.10 (m, 4H), 7.02 (t, 1H), 6.96 (d, 1H), 6.62 (t, 1H), 6.54 (dm, 1H), 6.52 (dm, 1H), 6.23 (br s, 1H), 5.46 (dm, 1H), 3.97/3.88 (dd+dd, 2H), 3.01 (m, 1H), 3.00/2.53 (dd+dd, 2H), 2.50-1.29 (m, 15H), 2.14 (m, 1H), 1.14 (d, 3H), 1.05 (d, 3H). HRMS calculated for C₃₅H₄₀N₂O₃FCl: 590.2712; found: 591.2780 (M+H).

### Example 689 (1r,2′S,4S)-4-(3-chloroanilino)-2′-[(2R)-3-{[(5R,8R)-8-fluoro-5-methyl-5,6,7,8-tetrahydroquinolin-4-yl]oxy}-2-methylpropyl]-2′,3′-dihydrospiro[cyclohexane-1,1′-indene]-4-carboxylic acid

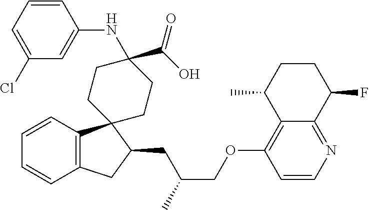 [SMILES: C[C@@H](COc1ccnc2c1[C@H](C)CC[C@H]2F)C[C@H]1Cc2ccccc2[C@]12CC[C@@](Nc1cccc(Cl)c1)(C(=O)O)CC2]

### Example 690 (1r,2′S,4S)-4-(3-chloroanilino)-2′-[(2R)-3-{[(5S,8S)-8-fluoro-5-methyl-5,6,7,8-tetrahydroquinolin-4-yl]oxy}-2-methylpropyl]-2′,3′-dihydrospiro[cyclohexane-1,1′-indene]-4-carboxylic acid

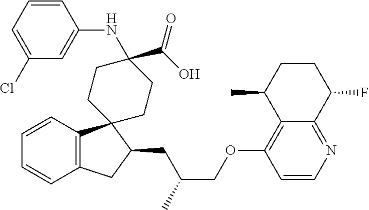 [SMILES: C[C@@H](COc1ccnc2c1[C@@H](C)CC[C@@H]2F)C[C@H]1Cc2ccccc2[C@]12CC[C@@](Nc1cccc(Cl)c1)(C(=O)O)CC2]

Using General procedure 31a and Preparation 10b1 as the appropriate indane and Example 689A as the appropriate aryl chloride, a mixture of diastereoisomers was obtained. The diastereoisomers were separated by chiral chromatography. Column: AD, 50×500 mm, 20 μm, Eluents: iPrOH/heptane. The diastereoisomer eluting earlier was hydrolyzed according to General Procedure 33a to obtain Example 689. ¹H NMR (500 MHz, DMSO-d₆) δ ppm: 12.70 (br s, 1H), 8.33 (d, 1H), 7.40-7.10 (m, 4H), 7.02 (t, 1H), 6.99 (d, 1H), 6.62 (t, 1H), 6.54 (dm, 1H), 6.52 (dm, 1H), 6.25 (br s, 1H), 5.35 (dm, 1H), 3.95/3.91 (dd+dd, 2H), 3.12 (m, 1H), 3.00/2.54 (dd+dd, 2H), 2.52-1.30 (m, 15H), 2.15 (m, 1H), 1.06 (d, 3H), 1.02 (d, 3H). HRMS calculated for C₃₅H₄₀N₂O₃FCl: 590.2712; found: 591.2782 (M+H).

The diastereoisomer eluting later was hydrolyzed according to General procedure 33a to obtain Example 690. ¹H NMR (500 MHz, DMSO-d₆) δ ppm: 12.69 (br s, 1H), 8.32 (d, 1H), 7.39-7.09 (m, 4H), 7.01 (t, 1H), 6.98 (d, 1H), 6.64 (t, 1H), 6.54 (dm, 1H), 6.49 (dm, 1H), 6.11 (br s, 1H), 5.36 (dm, 1H), 3.96/3.90 (dd+dd, 2H), 3.08 (m, 1H), 3.01/2.55 (dd+dd, 2H), 2.50-1.32 (m, 15H), 2.17 (m, 1H), 1.12 (d, 3H), 1.07 (d, 3H). HRMS calculated for C₃₅H₄₀N₂O₃FCl: 590.2712; found: 591.2776 (M+H).

### Example 691

### Example 691A 4-chloro-5-(difluoromethoxy)-5,6,7,8-tetrahydroquinoline

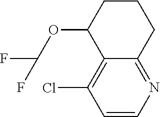 [SMILES: FC(F)OC1CCCc2nccc(Cl)c21]

To a solution of Example 671C (410 mg, 2.23 mmol, 1 eq) in MeCN (20 mL) was added CuI (85 mg, 0.45 mmol, 0.2 eq) and the mixture was heated to 50° C. under N₂. 2,2-difluoro-2-(fluorosulfonyl)acetic acid (0.46 mL, 4.47 mmol, 2 eq) was added and the mixture was heated at 60° C. for 1 h. The mixture was cooled to rt and partitioned between DCM and sat. aq. NaHCO₃ solution. The phases were separated, and the organic phase was washed with brine, dried (MgSO₄) and concentrated in vacuo. Purification by reverse phase automated flash chromatography at pH 4 (CombiFlash Rf, C18 30 g RediSep column) eluting with a gradient of 20-100% MeCN in water afforded a racemate, Example 691A, isolated as a red oil (140 mg, 0.6 mmol, 13%). LRMS calculated for C₁₀H₁₀F₂NO: 233; found: 234 (M+H). ¹H NMR (400 MHz, DMSO-d₆) δ ppm: 8.47 (d, J=5.2 Hz, 1H), 7.48-7.45 (m, 1H), 6.96 (dd, J=78.6, 73.3 Hz, 1H), 5.50-5.46 (m, 1H), 3.01-2.92 (m, 1H), 2.87-2.76 (m, 1H), 2.27-2.18 (m, 1H), 1.98-1.76 (m, 3H).

### Example 691B methyl (1r,2′S,4S)-4-(3-chloroanilino)-2′-[(2R)-3-{[5-(difluoromethoxy)-5,6,7,8-tetrahydroquinolin-4-yl]oxy}-2-methylpropyl]-2′,3′-dihydrospiro[cyclohexane-1,1′-indene]-4-carboxylate

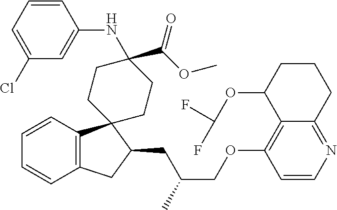 [SMILES: COC(=O)[C@]1(Nc2cccc(Cl)c2)CC[C@@]2(CC1)c1ccccc1C[C@@H]2C[C@@H](C)COc1ccnc2c1C(OC(F)F)CCC2]

A mixture of Preparation 10b1 (175 mg, 0.4 mmol, 1 eq), Example 691A (120 mg, 0.51 mmol, 1.3 eq), Cs₂CO₃ (323 mg, 0.99 mmol, 2.5 eq), bis[cinnamyl palladium (II) chloride] (10 mg, 19.8 μmol, 0.05 eq) and Josiphos SL-J009 (22 mg, 0.04 mmol, 0.1 eq) in toluene (2 mL) was sparged with N₂ and then heated at 90° C. for 48 h. The mixture was allowed to cool to rt and partitioned between DCM and sat. aq. NaHCO₃ solution. The organic phase was washed with brine, dried (PTFE phase separator) and concentrated in vacuo. Purification by reverse phase automated flash chromatography at pH 4 (CombiFlash Rf, C18 15.5 g Gold RediSep column) eluting with a gradient of 20-60% MeCN in water afforded a mixture of diastereoisomers, Example 691B, isolated as an off-white powder (23 mg, 0.04 mmol, 9%). LRMS calculated for C₃₆H₄₁ClF₂N₂O₄: 638; found: 639 (M+H). ¹H NMR (400 MHz, DMSO-d₆) δ ppm: 8.35-8.31 (m, 1H), 7.44-7.36 (m, 1H), 7.25-7.12 (m, 3H), 7.09-7.03 (m, 1H), 6.94-6.90 (m, 1H), 6.73/6.65 (dd, J=80, 74 Hz, 1H), 6.62-6.53 (m, 2H), 6.48-6.43 (m, 1H), 6.34/6.31 (s, 1H), 5.38-5.31 (m, 1H), 4.08-3.95 (m, 1H), 3.90-3.81 (m, 1H), 3.65 (s, 3H), 3.03-2.94 (m, 1H), 2.90-2.80 (m, 1H), 2.75-2.41 (m, 3H), 2.21-1.60 (m, 11H), 1.55-1.21 (m, 4H), 1.09-1.00 (m, 3H).

### Example 691 (1r,2′S,4S)-4-(3-chloroanilino)-2′-[(2R)-3-{[5-(difluoromethoxy)-5,6,7,8-tetrahydroquinolin-4-yl]oxy}-2-methylpropyl]-2′,3′-dihydrospiro[cyclohexane-1,1′-indene]-4-carboxylic acid

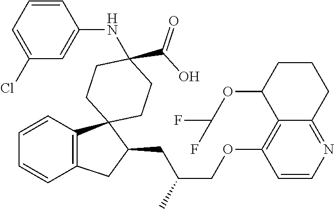 [SMILES: C[C@@H](COc1ccnc2c1C(OC(F)F)CCC2)C[C@H]1Cc2ccccc2[C@]12CC[C@@](Nc1cccc(Cl)c1)(C(=O)O)CC2]

Using General procedure 33a and Example 691B (23 mg, 0.04 mmol, 1 eq) as the appropriate ester, a mixture of diastereoisomers, Example 691, was obtained as a white powder (15.9 mg, 0.03 mmol, 71%). LRMS calculated for C₃₅H₃₉ClF₂N₂O₄: 624; found: 625 (M+H). ¹H NMR (400 MHz, DMSO-d₆) δ ppm: 8.36-8.31 (m, 1H), 7.44-7.35 (m, 1H), 7.25-7.12 (m, 3H), 7.09-7.03 (m, 1H), 6.95-6.90 (m, 1H), 6.73/6.65 (dd, J=80, 74 Hz, 1H), 6.64-6.60 (m, 1H), 6.57-6.52 (m, 2H), 5.38-5.31 (m, 1H), 4.08-3.95 (m, 1H), 3.89-3.82 (m, 1H), 3.03-2.94 (m, 1H), 2.90-2.80 (m, 1H), 2.76-2.40 (m, 3H), 2.21-1.61 (m, 11H), 1.55-1.23 (m, 4H), 1.09-1.00 (m, 3H).

### Example 692

### Example 692A (5S)-4-chloro-5-methyl-5,6,7,8-tetrahydroquinoline

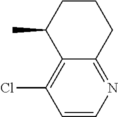

and

### Example 692B (5R)-4-chloro-5-methyl-5,6,7,8-tetrahydroquinoline

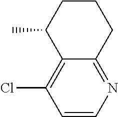

The enantiomers of Example 673C were separated by chiral chromatography. Column: AD, 100 mm×500 mm, 20 m. Eluent: EtOH/Heptane 1:99. The enantiomer eluting earlier was collected as Example 692A. The enantiomer eluting later was collected as Example 692B.

### Example 692C (5R)-4-chloro-5-methyl-1-oxo-5,6,7,8-tetrahydro-1λ⁵-quinoline

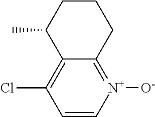

Example 692C was synthesized as described at Example 673D, using Example 692B instead of Example 673C.

### Example 692D (5R,8S)-4-chloro-5-methyl-5,6,7,8-tetrahydroquinolin-8-ol

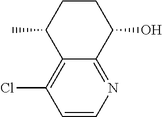

and

### Example 692E (5R,8R)-4-chloro-5-methyl-5,6,7,8-tetrahydroquinolin-8-ol

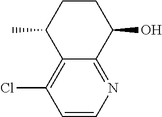

Example 692D and Example 692E were synthesized as described at Example 673E and Example 675A, using Example 692C instead of Example 673D.

Example 692D: LRMS calculated for C₁₀H₁₀ClNO: 197; found: 198 (M+H). ¹H NMR (400 MHz, DMSO-d₆) δ ppm: 8.39 (d, J=5.2 Hz, 1H), 7.41 (dd, J=5.2, 0.7 Hz, 1H), 5.25 (d, J=3.8 Hz, 1H), 4.57-4.50 (m, 1H), 3.23-3.14 (m, 1H), 2.06-1.81 (m, 3H), 1.77-1.68 (m, 1H), 1.26 (d, J=7.0 Hz, 3H).

### Example 692F (5R)-4-chloro-5-methyl-6,7-dihydroquinolin-8 (5H)-one

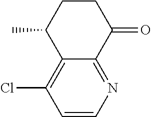

To a solution of Example 692D (403 mg, 2.04 mmol, 1 eq) in CHCl₃ (15 mL) was added MnO₂ (1.77 g, 20.4 mmol, 10 eq) and the mixture was stirred at rt for 18 h. The mixture was filtered through celite, washed with 10% MeOH in DCM and the filtrate was concentrated in vacuo to afford Example 692F as a brown solid (284 mg, 1.45 mmol, 71%). LRMS calculated for C₁₀H₁₂ClNO: 195; found: 196 (M+H). ¹H NMR (400 MHz, DMSO-d₆) δ ppm: 8.59 (d, J=5.0 Hz, 1H), 7.77 (d, J=5.0 Hz, 1H), 3.49-3.40 (m, 1H), 3.02 (ddd, J=17.8, 15.1, 5.3 Hz, 1H), 2.64-2.55 (m, 1H), 2.40-2.27 (m, 1H), 2.03-1.95 (m, 1H), 1.36 (d, J=7.1 Hz, 3H).

### Example 692G (5R)-4-chloro-8,8-difluoro-5-methyl-5,6,7,8-tetrahydroquinoline

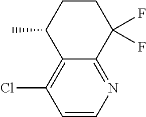

To a solution of Example 692F (285 mg, 1.46 mmol, 1 eq) in DCM (10 mL), cooled to 0° C., was added DAST (0.67 mL, 5.1 mmol, 3.5 eq). The mixture was slowly warmed to rt and stirred for 48 h under N₂. The mixture was cooled to 0° C. and quenched by the dropwise addition of 2 M aq. NaOH solution. The mixture was partitioned between DCM and 2 M aq. NaOH solution. The phases were separated, and the organic phase was washed with brine, dried (PTFE phase separator) and concentrated in vacuo. Purification by automated flash chromatography (CombiFlash Rf, Silica 12 g RediSep™ cartridge) eluting with a gradient of 0-29% EtOAc in heptane afforded Example 692G as a yellow oil (193 mg, 0.89 mmol, 61%). LRMS calculated for C₁₀H₁₀ClF₂N: 217; found: 218 (M+H). ¹H NMR (400 MHz, DMSO-d₆) δ ppm: 8.56 (d, J=5.1 Hz, 1H), 7.71 (dd, J=5.1, 1.4 Hz, 1H), 3.39-3.27 (m, 1H), 2.65-2.31 (m, 2H), 2.17-2.04 (m, 1H), 1.91-1.80 (m, 1H), 1.29 (d, J=7.1 Hz, 3H).

### Example 692H (5R)-8,8-difluoro-5-methyl-5,6,7,8-tetrahydroquinolin-4-ol

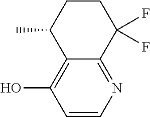

To an oven-dried flask was added Example 692G (190 mg, 0.87 mmol, 1 eq), K₂CO₃ (362 mg, 2.62 mmol, 3 eq), tBuXPhos (30 mg, 0.07 mmol, 0.08 eq) and Herrmann's catalyst (16 mg, 17.5 μmol, 0.02 eq) in DMF (5 mL) and water (0.5 mL). The mixture was sparged with N₂ and heated at 115° C. for 20 h. The mixture was allowed to cool to rt, filtered through celite, washed with DCM, then the filtrate was concentrated in vacuo. The residue was partitioned between DCM and 2 M aq. HCl solution, and the organic phase was discarded. The aq. phase was adjusted to pH 8 with solid NaHCO₃ and saturated with NaCl and the organics were extracted with DCM/iPrOH (3:1). The combined organic extracts were washed with brine, dried (MgSO₄) and concentrated in vacuo to afford Example 692H as a beige powder (157 mg, 0.79 mmol, 90%). LRMS calculated for C₁₀H₁₁F₂NO: 199; found: 200 (M+H).

### Example 692I methyl (1r,2′S,4S)-4-(3-chloroanilino)-2′-[(2R)-3-{[(5R)-8,8-difluoro-5-methyl-5,6,7,8-tetrahydroquinolin-4-yl]oxy}-2-methylpropyl]-2′,3′-dihydrospiro[cyclohexane-1,1′-indene]-4-carboxylate

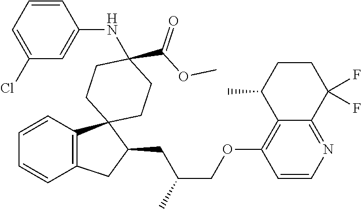 [SMILES: COC(=O)[C@]1(Nc2cccc(Cl)c2)CC[C@@]2(CC1)c1ccccc1C[C@@H]2C[C@@H](C)COc1ccnc2c1[C@H](C)CCC2(F)F]

Using General procedure 30a and Preparation 10b1 (150 mg, 0.34 mmol, 1 eq) as the appropriate indane and Example 692H (142 mg, 0.71 mmol, 2.1 eq) as the appropriate alcohol, Example 692I was obtained as a white powder (158 mg, 0.25 mmol, 75%). LRMS calculated for C₃₆H₄₁ClF₂N₂O₃: 622; found: 623 (M+H). ¹H NMR (400 MHz, DMSO-d₆) δ ppm: 8.41 (d, J=5.6 Hz, 1H), 7.43-7.35 (m, 1H), 7.24-7.12 (m, 3H), 7.10 (d, J=5.6 Hz, 1H), 7.05 (t, J=8.1 Hz, 1H), 6.61-6.54 (m, 2H), 6.48-6.42 (m, 1H), 6.34 (s, 1H), 4.04-3.90 (m, 2H), 3.66 (s, 3H), 3.21-3.10 (m, 1H), 3.01 (dd, J=15.6, 7.1 Hz, 1H), 2.59-2.23 (m, 4H), 2.21-1.85 (m, 7H), 1.79-1.68 (m, 2H), 1.55-1.44 (m, 2H), 1.42-1.31 (m, 2H), 1.11 (d, J=7.0 Hz, 3H), 1.06 (d, J=6.7 Hz, 3H).

### Example 692 (1r,2′S,4S)-4-(3-chloroanilino)-2′-[(2R)-3-{[(5R)-8,8-difluoro-5-methyl-5,6,7,8-tetrahydroquinolin-4-yl]oxy}-2-methylpropyl]-2′,3′-dihydrospiro[cyclohexane-1,1′-indene]-4-carboxylic acid

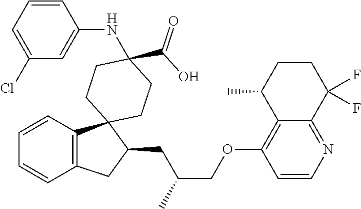 [SMILES: C[C@@H](COc1ccnc2c1[C@H](C)CCC2(F)F)C[C@H]1Cc2ccccc2[C@]12CC[C@@](Nc1cccc(Cl)c1)(C(=O)O)CC2]

Using General procedure 33a and Example 692I(158 mg, 0.25 mmol, 1 eq) as the appropriate ester (and heating at 75° C. for 7H), Example 692 was obtained, isolated as a white powder (107 mg, 0.18 mmol, 70%). LRMS calculated for C₃₅H₃₉ClF₂N₂O₃: 608; found: 609 (M+H). ¹H NMR (400 MHz, DMSO-d₆) δ ppm: 8.41 (d, J=5.5 Hz, 1H), 7.43-7.34 (m, 1H), 7.24-7.12 (m, 3H), 7.10 (d, J=5.5 Hz, 1H), 7.05 (t, J=8.1 Hz, 1H), 6.62 (t, J=2.1 Hz, 1H), 6.57-6.51 (m, 2H), 4.04-3.90 (m, 2H), 3.20-3.10 (m, 1H), 3.01 (dd, J=15.6, 7.1 Hz, 1H), 2.60-2.23 (m, 4H), 2.20-1.81 (m, 7H), 1.79-1.67 (m, 2H), 1.55-1.44 (m, 2H), 1.44-1.31 (m, 2H), 1.11 (d, J=6.9 Hz, 3H), 1.06 (d, J=6.6 Hz, 3H).

### Example 693

### Example 693A 4-chloro-5,5-difluoro-5,6,7,8-tetrahydroquinoline

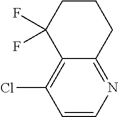 [SMILES: FC1(F)CCCc2nccc(Cl)c21]

To a solution of Example 671B (0.23 g, 1.28 mmol, 1 eq) in DCM (10 mL), cooled to 0° C., was added DAST (0.42 mL, 3.2 mmol, 2.5 eq) dropwise. The mixture was stirred at 0° C. for 45 min, then slowly allowed to warm to rt and stirred for 18 h. Further DAST (0.42 mL, 3.2 mmol, 2.5 eq) was added and the mixture was stirred at rt for 18 h, then heated at reflux for 4 h. The mixture was cooled to 0° C. and quenched by the dropwise addition of 2 M aq. NaOH solution and then the mixture was stirred for 10 min. The phases were separated, and the aq. phase was extracted with DCM. The combined organic extracts were washed with brine, dried (MgSO₄) and concentrated in vacuo. Purification by automated flash chromatography (CombiFlash Rf, Silica 4 g RediSep™ cartridge) eluting with a gradient of 0-30% EtOAc in heptane afforded Example 693A, isolated as a yellow solid (55 mg, 0.27 mmol, 21%). LRMS calculated for C₉H₈ClF₂N: 203; found: 204 (M+H). ¹H NMR (400 MHz, DMSO-d₆) δ ppm: 8.55 (dt, J=5.2, 1.0 Hz, 1H), 7.54 (d, J=5.2 Hz, 1H), 3.03-2.95 (m, 2H), 2.45-2.32 (m, 2H), 1.95-1.87 (m, 2H).

### Example 693B methyl (1r,2′S,4S)-4-(3-chloroanilino)-2′-{(2R)-3-[(5,5-difluoro-5,6,7,8-tetrahydroquinolin-4-yl)oxy]-2-methylpropyl}-2′,3′-dihydrospiro[cyclohexane-1,1′-indene]-4-carboxylate

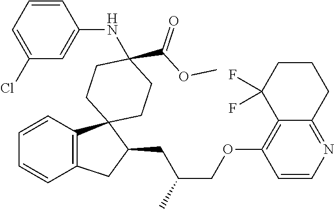 [SMILES: COC(=O)[C@]1(Nc2cccc(Cl)c2)CC[C@@]2(CC1)c1ccccc1C[C@@H]2C[C@@H](C)COc1ccnc2c1C(F)(F)CCC2]

A mixture of Preparation 10b1 (93 mg, 0.21 mmol, 1 eq), Example 693A (50 mg, 0.25 mmol, 1.17 eq), Cs₂CO₃ (171 mg, 0.53 mmol, 2.5 eq), bis[cinnamyl palladium (II) chloride] (5 mg, 10 μmol, 0.05 eq) and Josiphos SL-J009 (12 mg, 20 umol, 0.1 eq) in toluene (1 mL) was sparged with N₂ and then heated at 90° C. for 24 h. The mixture was allowed to cool to rt and partitioned between DCM and sat. aq. NaHCO₃ solution. The phases were separated, and the organic phase was washed with brine, dried (MgSO₄) and concentrated in vacuo. Purification by automated flash chromatography (CombiFlash Rf, Silica 4 g RediSep™ cartridge) eluting with a gradient of 0-50% EtOAc in heptane afforded Example 693B as a yellow glass (70 mg, 0.11 mmol, 55%). LRMS calculated for C₃₅H₃₉ClF₂N₂O₃: 608; found: 609 (M+H). ¹H NMR (400 MHz, CDCl₃) δ ppm: 8.40 (d, J=5.7 Hz, 1H), 7.52-7.44 (m, 1H), 7.28-7.10 (m, 3H), 7.03 (t, J=8.0 Hz, 1H), 6.72-6.67 (m, 2H), 6.61 (t, J=2.1 Hz, 1H), 6.47-6.42 (m, 1H), 4.06 (s, 1H), 3.98-3.89 (m, 2H), 3.74 (s, 3H), 3.01 (dd, J=15.5, 7.3 Hz, 1H), 2.97-2.90 (m, 2H), 2.68-2.56 (m, 2H), 2.37-2.03 (m, 6H), 2.03-1.72 (m, 5H), 1.70-1.38 (m, 4H), 1.10 (d, J=6.7 Hz, 3H).

### Example 693 (1r,2′S,4S)-4-(3-chloroanilino)-2′-{(2R)-3-[(5,5-difluoro-5,6,7,8-tetrahydroquinolin-4-yl)oxy]-2-methylpropyl}-2′,3′-dihydrospiro[cyclohexane-1,1′-indene]-4-carboxylic acid

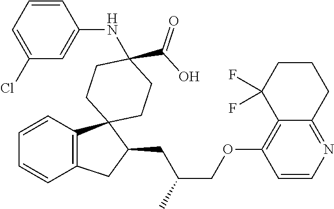 [SMILES: C[C@@H](COc1ccnc2c1C(F)(F)CCC2)C[C@H]1Cc2ccccc2[C@]12CC[C@@](Nc1cccc(Cl)c1)(C(=O)O)CC2]

Using General procedure 33a and Example 693B (70 mg, 0.11 mmol, 1 eq) as the appropriate ester (and heating at 75° C. for 7H), Example 693 was isolated as an off-white solid (32 mg, 0.05 mmol, 47%). LRMS calculated for C₃₄H₃₇ClF₂N₂O₃: 594; found: 595 (M+H). ¹H NMR (400 MHz, DMSO-d₆) δ ppm: 8.39 (d, J=5.8 Hz, 1H), 7.44-7.35 (m, 1H), 7.24-7.11 (m, 3H), 7.05 (t, J=8.1 Hz, 1H), 7.00 (d, J=5.9 Hz, 1H), 6.62 (t, J=2.1 Hz, 1H), 6.58-6.51 (m, 2H), 4.00 (dd, J=9.4, 5.8 Hz, 1H), 3.93 (dd, J=9.4, 6.2 Hz, 1H), 2.98 (dd, J=15.6, 7.1 Hz, 1H), 2.89-2.79 (m, 2H), 2.60-2.39 (m, 2H), 2.33-2.17 (m, 2H), 2.17-1.79 (m, 8H), 1.79-1.67 (m, 1H), 1.55-1.42 (m, 2H), 1.40-1.28 (m, 2H), 1.04 (d, J=6.6 Hz, 3H).

### Example 694 and Example 695

### Example 694A N-[3-(2,2,2-trifluoroethyl)phenyl]acetamide

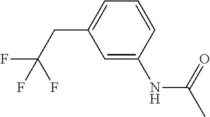

AcCl (1.12 mL, 15.7 mmol, 1.1 eq) was added dropwise to a solution of 3-(2,2,2-trifluoroethyl)aniline (2.5 g, 14.27 mmol, 1 eq) and TEA (3.97 mL, 28.55 mmol, 2 eq) in DCM (40 mL) cooled to 0° C. under N₂ and stirred for 2 h. The mixture was quenched with water (60 mL), 2 M aq. HCl solution (5 mL) was added and the mixture was extracted with DCM. The organic extracts were combined, washed with sat. aq. NH₄Cl solution, dried (MgSO₄) and concentrated in vacuo to afford Example 694A as a cream powder (3.03 g, 13.97 mmol, 98%). LRMS calculated for C₁₀H₁₀F₃NO: 217; found: 218 (M+H). ¹H NMR (400 MHz, DMSO-d₆) δ ppm: 10.00 (s, 1H), 7.63-7.58 (m, 1H), 7.58-7.52 (m, 1H), 7.29 (t, J=7.8 Hz, 1H), 7.04-6.99 (m, 1H), 3.61 (q, J=11.6 Hz, 2H), 2.04 (s, 3H).

### Example 694B N-[2-chloro-5-(2,2,2-trifluoroethyl)phenyl]acetamide

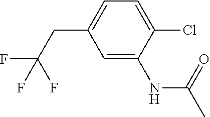 [SMILES: CC(=O)Nc1cc(CC(F)(F)F)ccc1Cl]

A mixture of Example 694A (3.03 g, 13.95 mmol, 1 eq), PTSA (1.33 g, 6.98 mmol, 0.5 eq), NCS (2.24 g, 16.74 mmol, 1.2 eq) and Pd(OAc)₂ (157 mg, 0.7 mmol, 0.05 eq) in toluene (60 mL) was stirred under air at 50° C. for 3 h and then at rt for 18 h. The mixture was partitioned between EtOAc and sat. aq. NaHCO₃ solution. The phases were separated, and the organic phase was washed with water, dried (MgSO₄) and concentrated in vacuo. The residue was triturated with DCM and the solids were collected by filtration to afford Example 694B as an off-white powder (3.07 g, 12.2 mmol, 87%). LRMS calculated for C₁₀H₉ClF₃NO: 251; found: 252 (M+H). ¹H NMR (400 MHz, DMSO-d₆) δ ppm: 9.57 (s, 1H), 7.77-7.69 (m, 1H), 7.51 (d, J=8.2 Hz, 1H), 7.22-7.14 (m, 1H), 3.68 (q, J=11.6 Hz, 2H), 2.10 (s, 3H).

### Example 694C 2-chloro-5-(2,2,2-trifluoroethyl)aniline

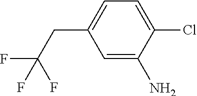 [SMILES: Nc1cc(CC(F)(F)F)ccc1Cl]

Cc. aq. HCl solution (12 M, 5 mL) was added slowly to a stirred solution of Example 694B (3.07 g, 12.2 mmol, 1 eq) in 1,4-dioxane (50 mL) and the mixture was heated at 85° C. for 5 h. The mixture was allowed to cool to rt, concentrated in vacuo to half the volume and then partitioned between DCM and water. The phases were separated, and the organic phase was washed with 2 M aq. NaOH solution, dried (MgSO₄) and concentrated in vacuo to afford Example 694C as an orange oil (2.3 g, 10.99 mmol, 90%). ¹H NMR (400 MHz, DMSO-d₆) δ ppm: 7.17 (d, J=8.2 Hz, 1H), 6.76 (d, J=2.1 Hz, 1H), 6.51 (dd, J=8.2, 2.1 Hz, 1H), 5.44 (s, 2H), 3.49 (q, J=11.6 Hz, 2H).

### Example 694D 5-{[2-chloro-5-(2,2,2-trifluoroethyl)anilino]methylidene}-2,2-dimethyl-1,3-dioxane-4,6-dione

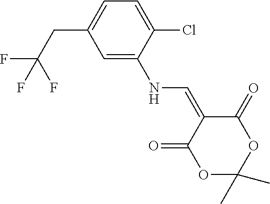 [SMILES: CC1(C)OC(=O)C(=CNc2cc(CC(F)(F)F)ccc2Cl)C(=O)O1]

5-(Methoxymethylene)-2,2-dimethyl-1,3-dioxane-4,6-dione (2.25 g, 12.07 mmol, 1.1 eq) was added to a solution of Example 694C (2.3 g, 10.97 mmol, 1 eq) in EtOH (50 mL) and the suspension was stirred at rt for 3 h. The precipitate was collected by filtration, washed with EtOH and dried to afford Example 694D as a cream powder (3.26 g, 8.96 mmol, 82%). ¹H NMR (400 MHz, DMSO-d₆) δ ppm: 11.58 (d, J=13.8 Hz, 1H), 8.79 (d, J=13.8 Hz, 1H), 8.03 (d, J=1.9 Hz, 1H), 7.66 (d, J=8.3 Hz, 1H), 7.32-7.27 (m, 1H), 3.75 (q, J=11.4 Hz, 2H), 1.70 (s, 6H).

### Example 694E 8-chloro-5-(2,2,2-trifluoroethyl)quinolin-4-ol

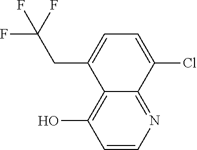 [SMILES: Oc1ccnc2c(Cl)ccc(CC(F)(F)F)c12]

A suspension of Example 694D (3.26 g, 8.96 mmol, 1 eq) in Ph₂O (30 mL) was sparged with N₂, and then placed in heating block, pre-heated to 260° C., and stirred rapidly at 250-260° C. for 30 min. The mixture was allowed to cool to rt, heptane (50 mL) was added and the mixture was stirred for 10 min. The precipitate was collected by filtration, washed with heptane and the solids were dried in vacuo to afford Example 694E as a brown powder (1.55 g, 5.92 mmol, 66%). LRMS calculated for C₁₁H₇ClF₃NO: 261; found: 262 (M+H). ¹H NMR (400 MHz, DMSO-d₆) δ ppm: 11.32 (br s, 1H), 7.86 (d, J=8.0 Hz, 1H), 7.82 (d, J=7.5 Hz, 1H), 7.24 (d, J=8.0 Hz, 1H), 6.13 (d, J=7.5 Hz, 1H), 4.69 (q, J=11.4 Hz, 2H).

### Example 694F (5R)-5-(2,2,2-trifluoroethyl)-5,6,7,8-tetrahydroquinolin-4-ol

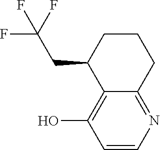 [SMILES: Oc1ccnc2c1[C@@H](CC(F)(F)F)CCC2]

and

### Example 695A (5S)-5-(2,2,2-trifluoroethyl)-5,6,7,8-tetrahydroquinolin-4-ol

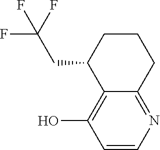 [SMILES: Oc1ccnc2c1[C@H](CC(F)(F)F)CCC2]

Example 694E (1.08 g, 4.13 mmol, 1 eq) was added portionwise to TFA (20 mL, 268.37 mmol, 65.01 eq), cooled to 0° C. The mixture was allowed to warm to rt, placed under a N₂ atmosphere and PtO₂ (47 mg, 0.21 mmol, 0.05 eq) was added. The mixture was evacuated and flushed with H₂ and then heated at 57° C. with vigorous stirring for 24 h under a H₂ atmosphere. The mixture was allowed to cool to rt, filtered through celite, washed with DCM/MeOH (3:1). The solvents were removed in vacuo and the residue was partitioned between DCM and 2 M aq. NaOH solution. The phases were separated, and the aq. phase was extracted with DCM/IPA (4:1). The combined organic extracts were dried (MgSO₄) and concentrated in vacuo. The residue was triturated with Et₂O and the solids were collected by filtration. The enantiomers were separated by chiral chromatography. Column: IC, 50×500 mm, 20 μm, Eluents: EtOH/heptane. The enantiomer eluting earlier was collected as Example 694F. ¹H NMR (400 MHz, DMSO-d₆) δ ppm: 11.23 (br s, 1H), 7.52 (d, 1H), 5.98 (d, 1H), 3.08 (m, 1H), 2.63/2.16 (m+m, 2H), 2.50 (m, 2H), 1.84/1.59 (m+m, 2H), 1.73 (m, 2H). HRMS calculated for C₁₁H₁₂NOF₃: 231.0871; found: 232.0945 (M+H).

The enantiomer eluting later was collected as Example 695A. HRMS calculated for C₁₁H₁₂NOF₃: 231.0871; found: 232.0948 (M+H).

### Example 694 (1r,2′S,4S)-4-(3-chloroanilino)-2′-[(2R)-2-methyl-3-{[(5R)-5-(2,2,2-trifluoroethyl)-5,6,7,8-tetrahydroquinolin-4-yl]oxy}propyl]-2′,3′-dihydrospiro[cyclohexane-1,1′-indene]-4-carboxylic acid

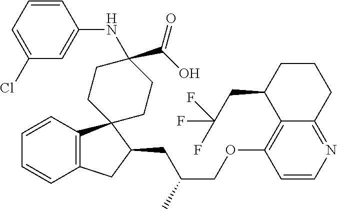 [SMILES: C[C@@H](COc1ccnc2c1[C@@H](CC(F)(F)F)CCC2)C[C@H]1Cc2ccccc2[C@]12CC[C@@](Nc1cccc(Cl)c1)(C(=O)O)CC2]

Using General procedure 32 and Preparation 10b1 as the appropriate indane and Example 694F as the appropriate alcohol Example 694 was obtained. ¹H NMR (500 MHz, DMSO-d₆) δ ppm: 12.65 (br s, 1H), 8.21 (d, 1H), 7.40-7.10 (m, 4H), 7.03 (t, 1H), 6.86 (d, 1H), 6.62 (t, 1H), 6.53 (dm, 1H), 6.52 (dm, 1H), 6.18 (br s, 1H), 3.95/3.86 (dd+dd, 2H), 3.29 (m, 1H), 2.97/2.53 (dd+dd, 2H), 2.79/2.68 (m+m, 2H), 2.50-1.30 (m, 12H), 2.44/2.37 (m+m, 2H), 2.15 (m, 1H), 2.01 (m, 1H), 1.46/1.28 (m+m, 2H), 1.03 (d, 3H). HRMS calculated for C₃₆H₄₀N₂O₃F₃Cl: 640.2679; found: 641.2742 (M+H).

### Example 695 (1r,2′S,4S)-4-(3-chloroanilino)-2′-[(2R)-2-methyl-3-{[(5S)-5-(2,2,2-trifluoroethyl)-5,6,7,8-tetrahydroquinolin-4-yl]oxy}propyl]-2′,3′-dihydrospiro[cyclohexane-1,1′-indene]-4-carboxylic acid

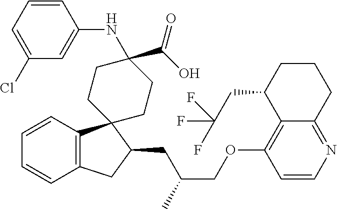 [SMILES: C[C@@H](COc1ccnc2c1[C@H](CC(F)(F)F)CCC2)C[C@H]1Cc2ccccc2[C@]12CC[C@@](Nc1cccc(Cl)c1)(C(=O)O)CC2]

Using General procedure 32 and Preparation 10b1 as the appropriate indane and Example 695A as the appropriate alcohol Example 695 was obtained. ¹H NMR (500 MHz, DMSO-d₆) δ ppm: 12.70 (br s, 1H), 8.22 (d, 1H), 7.43-7.10 (m, 4H), 7.02 (t, 1H), 6.86 (d, 1H), 6.61 (t, 1H), 6.52 (m, 2H), 6.16 (br s, 1H), 4.01/3.84 (dd+dd, 2H), 3.33 (m, 1H), 2.97/2.52 (dd+dd, 2H), 2.80/2.70 (m+m, 2H), 2.50-1.25 (m, 12H), 2.44/2.36 (m+m, 2H), 2.12 (m, 1H), 2.01 (m, 1H), 1.45/1.37 (m+m, 2H), 1.04 (d, 3H). HRMS calculated for C₃₆H₄₀N₂O₃F₃Cl: 640.2679; found: 641.2754 (M+H).

### Example 696 and Example 697

### Example 696A rac-(5R,8R)-4-chloro-5-ethyl-8-fluoro-5,6,7,8-tetrahydroquinoline

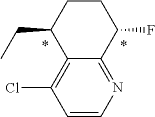

DAST (0.59 mL, 4.46 mmol, 1.5 eq) was added dropwise to a solution of Example 6781 (629 mg, 2.97 mmol, 1 eq) in DCM (30 mL), cooled to 0° C. under N₂ and then the mixture was slowly allowed to warm to rt over 3 h. The mixture was cooled to 0° C. and quenched by the slow addition of sat. aq. NH₄Cl solution (5 mL), followed by the careful dropwise addition of 2 M aq. NaOH solution (20 mL). The mixture was extracted with DCM and the organic extracts were combined, dried (MgSO₄) and concentrated in vacuo. Purification by automated flash chromatography (Combiflash Rf, Silica 24 g Gold RediSep™ cartridge) eluting with a gradient of 0-40% EtOAc in heptane afforded a racemate, Example 696A, isolated as a colourless oil (439 mg, 2.05 mmol, 69%). LRMS calculated for C_{II}H₁₃ClFN: 213; found: 214 (M+H). ¹H NMR (400 MHz, DMSO-d₆) δ ppm: 8.49-8.46 (m, 1H), 7.60 (dd, J=5.2, 2.1 Hz, 1H), 5.55-5.39 (m, 1H), 2.99-2.90 (m, 1H), 2.31-2.07 (m, 2H), 1.93-1.81 (m, 2H), 1.62-1.48 (m, 1H), 1.48-1.33 (m, 1H), 1.02 (t, J=7.4 Hz, 3H).

### Example 696B (5S,8S)-5-ethyl-8-fluoro-5,6,7,8-tetrahydroquinolin-4-ol

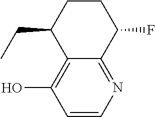

and

### Example 696C (5R,8R)-5-ethyl-8-fluoro-5,6,7,8-tetrahydroquinolin-4-ol

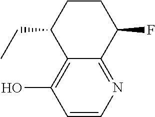

A mixture of Example 696A (439 mg, 2.05 mmol, 1 eq) and K₂CO₃ (852 mg, 6.16 mmol, 3 eq) were combined in DMF (9 mL) and water (0.9 mL) and then sparged with N₂. tBuXPhos (70 mg, 0.16 mmol, 0.08 eq) and Herrmann's catalyst (39 mg, 41.09 μmol, 0.02 eq) were added, the mixture was sparged with N₂ and then heated at 115° C. for 18 h. The mixture was allowed to cool to rt, filtered through celite, washed with 10% DCM/MeOH and the filtrate was concentrated in vacuo. Purification by reverse phase automated flash chromatography at pH 4 (Combiflash Rf, C18 30 g Gold RediSep™ cartridge) eluting with a gradient of 10-100% MeCN in water afforded a racemate, isolated as a white powder. The enantiomers were separated by chiral chromatography. Column: IC, 50×500 mm, 20 μm, Eluents: EtOH/heptane+0.005% DEA. The enantiomer eluting earlier was collected as Example 696B. ¹H NMR (500 MHz, DMSO-d₆) δ ppm: 8.55 (d, 1H), 7.30 (d, 1H), 5.71 (dt, 1H), 2.91 (m, 1H), 2.21/2.14 (m+m, 2H), 1.87/1.79 (dm+tm, 2H), 1.64/1.34 (m+m, 2H), 0.97 (t, 3H). HRMS calculated for C₁₁H₁₄NOF: 195.1059; found: 196.1137 (M+H).

The enantiomer eluting later was collected as Example 696C. HRMS calculated for C₁₁H₁₄NOF: 195.1059; found: 196.1130 (M+H).

### Example 696 (1r,2′S,4S)-4-(3-chloroanilino)-2′-[(2R)-3-{[(5S,8S)-5-ethyl-8-fluoro-5,6,7,8-tetrahydroquinolin-4-yl]oxy}-2-methylpropyl]-2′,3′-dihydrospiro[cyclohexane-1,1′-indene]-4-carboxylic acid

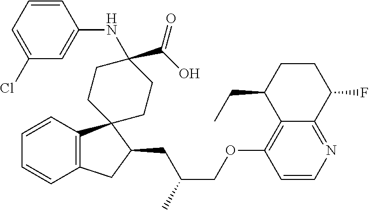 [SMILES: CC[C@H]1CC[C@H](F)c2nccc(OC[C@H](C)C[C@H]3Cc4ccccc4[C@]34CC[C@@](Nc3cccc(Cl)c3)(C(=O)O)CC4)c21]

Using General procedure 32 and Preparation 10b1 as the appropriate indane and Example 696B as the appropriate alcohol Example 696 was obtained. ¹H NMR (500 MHz, DMSO-d₆) δ ppm: 8.33 (d, 1H), 7.40-7.10 (m, 4H), 7.03 (t, 1H), 6.99 (dd, 1H), 6.63 (t, 1H), 6.53 (m, 2H), 6.19 (br s, 1H), 5.36 (dm, 1H), 3.98/3.89 (dd+dd, 2H), 3.01/2.55 (dd+dd, 2H), 2.77 (m, 1H), 2.50-1.31 (m, 12H), 2.15 (m, 1H), 2.02 (m, 1H), 1.52/1.27 (m+m, 2H), 1.43/1.37 (m+m, 2H), 1.06 (d, 3H), 0.87 (t, 3H). HRMS calculated for C₃₆H₄₂N₂O₃FCl: 604.2868; found: 605.2933 (M+H).

### Example 697 (1r,2′S,4S)-4-(3-chloroanilino)-2′-[(2R)-3-{[(5R,8R)-5-ethyl-8-fluoro-5,6,7,8-tetrahydroquinolin-4-yl]oxy}-2-methylpropyl]-2′,3′-dihydrospiro[cyclohexane-1,1′-indene]-4-carboxylic acid

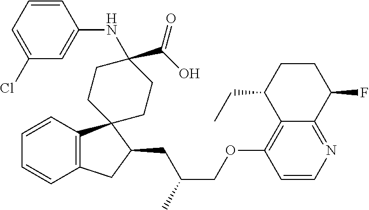 [SMILES: CC[C@@H]1CC[C@@H](F)c2nccc(OC[C@H](C)C[C@H]3Cc4ccccc4[C@]34CC[C@@](Nc3cccc(Cl)c3)(C(=O)O)CC4)c21]

Using General procedure 32 and Preparation 10b1 as the appropriate indane and Example 696C as the appropriate alcohol Example 697 was obtained. ¹H NMR (500 MHz, DMSO-d₆) δ ppm: 8.33 (d, 1H), 7.43-7.10 (m, 4H), 7.02 (t, 1H), 7.00 (dd, 1H), 6.61 (t, 1H), 6.52 (m, 2H), 6.20 (br s, 1H), 5.36 (dm, 1H), 3.93 (d, 2H), 3.01/2.54 (dd+dd, 2H), 2.79 (m, 1H), 2.50-1.29 (m, 12H), 2.13 (m, 1H), 2.02 (m, 1H), 1.54/1.37 (m+m, 2H), 1.47/1.2 (m+m, 2H), 1.04 (d, 3H), 0.78 (t, 3H). HRMS calculated for C₃₆H₄₂N₂O₃FCl: 604.2868; found: 605.2934 (M+H).

### Example 698 and Example 699

### Example 698A 5-{[2-chloro-5-(trifluoromethyl)anilino]methylidene}-2,2-dimethyl-1,3-dioxane-4,6-dione

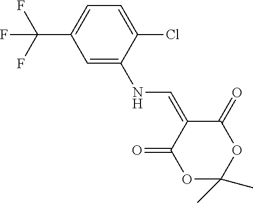 [SMILES: CC1(C)OC(=O)C(=CNc2cc(C(F)(F)F)ccc2Cl)C(=O)O1]

2-chloro-5-(trifluoromethyl)aniline (3.91 g, 20 mmol) and Meldrum's acid (4.10 g, 22 mmol) were suspended in dry EtOH (90 mL). The mixture was stirred at rt for 1 h. Then the mixture was concentrated under reduced pressure and the residue was suspended in DIPE (60 mL) and filtered. The solid was washed with DIPE (20 mL) and dried to obtain Example 698A. ¹H NMR (500 MHz, DMSO-d₆) δ ppm: 11.66 (d, 1H), 8.98 (d, 1H), 8.41 (s, 1H), 7.85 (d, 1H), 7.60 (dd, 1H), 1.70 (s, 6H).

### Example 698B (5S)-5-(trifluoromethyl)-5,6,7,8-tetrahydroquinolin-4-ol

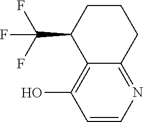 [SMILES: Oc1ccnc2c1[C@@H](C(F)(F)F)CCC2]

and

### Example 698C (5R)-5-(trifluoromethyl)-5,6,7,8-tetrahydroquinolin-4-ol

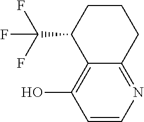 [SMILES: Oc1ccnc2c1[C@H](C(F)(F)F)CCC2]

Example 698A (500 mg, 1.43 mmol) was added to the stirred Eaton's reagent (1.4 mL, 1.5 g/mL P₂O₅ in methane sulfonic acid) at rt, then the mixture was stirred at 65° C. for 2 h, then at 85° C. for 2 h. KHCO₃ (10 g) was dissolved in water (40 mL) and cooled to 0° C. The crude reaction mixture was added dropwise into the KHCO₃ solution. The mixture was extracted with EtOAc, dried over MgSO₄, filtered and the filtrate was concentrated under reduced pressure. Then it was dissolved in AcOH (6 mL) and MeOH (2 mL). Pd/C (0.045 g) and NaOAc (0.225 g, 2.73 mmol) were added to the mixture. The flask was evacuated and backfilled with N₂ (×3), then evacuated and filled with H₂. The reaction mixture was stirred at 70° C. overnight. Then it was filtered through celite and the filtrate was concentrated under reduced pressure. The residue was dissolved in AcOH (6 mL). PtO₂ (0.040 g) and TFA (0.42 mL) were added to the mixture. The flask was evacuated and backfilled with N₂ (×3), then evacuated and filled with H₂. The reaction mixture was stirred at 70° C. overnight. The reaction mixture was filtered through celite and the filtrate was concentrated under reduced pressure. The crude product was purified via flash chromatography using DCM and MeOH as eluents to obtain a racemate. ¹H NMR (500 MHz, DMSO-d₆) δ ppm: 12.02 (br s, 1H), 7.73 (d, 1H), 6.24 (d, 1H), 3.88 (m, 1H), 2.68 (m, 2H), 2.07/1.67 (m+m, 2H), 1.86/1.82 (m+m, 2H).

The enantiomers were separated by chiral chromatography. Column: AS-V, 100×500 mm, 20 μm, Eluents: EtOH/heptane. The enantiomer eluting earlier was collected as Example 698B. The enantiomer eluting later was collected as Example 698C.

### Example 698 (1r,2′S,4S)-4-(3-chloroanilino)-2′-[(2R)-2-methyl-3-{[(5S)-5-(trifluoromethyl)-5,6,7,8-tetrahydroquinolin-4-yl]oxy}propyl]-2′,3′-dihydrospiro[cyclohexane-1,1′-indene]-4-carboxylic acid

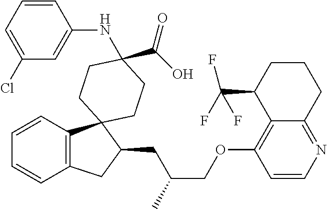 [SMILES: C[C@@H](COc1ccnc2c1[C@@H](C(F)(F)F)CCC2)C[C@H]1Cc2ccccc2[C@]12CC[C@@](Nc1cccc(Cl)c1)(C(=O)O)CC2]

Using General procedure 32 and Preparation 10b1 as the appropriate indane and Example 698B as the appropriate alcohol Example 698 was obtained. ¹H NMR (400 MHz, DMSO-d₆) δ ppm: 12.69 (br s, 1H), 8.28 (d, 1H), 7.38-7.10 (m, 4H), 7.04 (t, 1H), 6.88 (d, 1H), 6.63 (t, 1H), 6.55 (dm, 1H), 6.54 (dm, 1H), 6.23 (br s, 1H), 3.92/3.83 (dd+dd, 2H), 3.88 (m, 1H), 3.00/2.55 (dd+dd, 2H), 2.85/2.73 (m+m, 2H), 2.50-1.38 (m, 12H), 2.16 (m, 1H), 1.99 (m, 1H), 1.39/1.30 (m+m, 2H), 1.04 (d, 3H). HRMS calculated for C₃₅H₃₈N₂O₃F₃Cl: 626.2523; found: 627.2600 (M+H).

### Example 699 (1r,2′S,4S)-4-(3-chloroanilino)-2′-[(2R)-2-methyl-3-{[(5R)-5-(trifluoromethyl)-5,6,7,8-tetrahydroquinolin-4-yl]oxy}propyl]-2′,3′-dihydrospiro[cyclohexane-1,1′-indene]-4-carboxylic acid

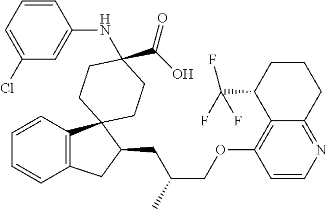 [SMILES: C[C@@H](COc1ccnc2c1[C@H](C(F)(F)F)CCC2)C[C@H]1Cc2ccccc2[C@]12CC[C@@](Nc1cccc(Cl)c1)(C(=O)O)CC2]

Using General procedure 32 and Preparation 10b1 as the appropriate indane and Example 698C as the appropriate alcohol Example 699 was obtained. ¹H NMR (400 MHz, DMSO-d₆) δ ppm: 12.64 (br s, 1H), 8.29 (d, 1H), 7.43-7.09 (m, 4H), 7.03 (t, 1H), 6.90 (d, 1H), 6.61 (t, 1H), 6.53 (m, 2H), 6.24 (br s, 1H), 3.97/3.86 (dd+dd, 2H), 3.95 (m, 1H), 2.99/2.52 (dd+dd, 2H), 2.86/2.77 (m+m, 2H), 2.51-1.26 (m, 12H), 2.11 (m, 1H), 1.97 (m, 1H), 1.47/1.32 (m+m, 2H), 1.03 (d, 3H). HRMS calculated for C₃₅H₃₈N₂O₃F₃Cl: 626.2523; found: 627.2596 (M+H).

### Example 700 and Example 701

### Example 700A 2,2-dimethyl-5-{[(3-oxocyclohex-1-en-1-yl)amino]methylidene}-1,3-dioxane-4,6-dione

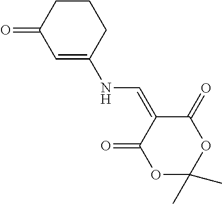 [SMILES: CC1(C)OC(=O)C(=CNC2=CC(=O)CCC2)C(=O)O1]

5-(Methoxymethylene)-2,2-dimethyl-1,3-dioxane-4,6-dione (20.8 g, 112 mmol, 1.13 eq) was slowly added to a stirred suspension of 3-aminocyclohex-2-en-1-one (11 g, 99 mmol, 1 eq) in MeCN (100 mL) and the suspension was stirred for 72 h at rt. The suspension was filtered, and the solids were washed with MeCN and dried in vacuo to afford Example 700A as a beige solid (10 g, 37.7 mmol, 38%). The filtrate was concentrated in vacuo, triturated with MeOH and the mixture was placed in a freezer for 1 h. The solids were collected by filtration, washed with cold MeOH and dried in vacuo to afford additional Example 700A, isolated as a yellow powder (10 g, 37.7 mmol, 38%). LRMS calculated for C₁₁H₁₅NO₅: 265; found: 266 (M+H). ¹H NMR (400 MHz, DMSO-d₆) δ ppm: 10.92 (d, J=14.0 Hz, 1H), 8.34 (d, J=14.0 Hz, 1H), 6.15-6.10 (m, 1H), 2.72 (t, J=6.1 Hz, 2H), 2.32-2.25 (m, 2H), 2.03-1.94 (m, 2H), 1.68 (s, 6H).

### Example 700B 7,8-dihydroquinoline-4,5 (1H,6H)-dione

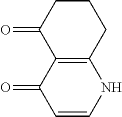 [SMILES: O=C1CCCc2[nH]ccc(=O)c21]

Example 700A (7.5 g, 28.3 mmol, 1 eq) was suspended in Ph₂O (51.1 g, 40 mL, 300 mmol) and the mixture was then placed in a heating block, pre-heated to 260° C. and stirred rapidly at 250-260° C. for 20 min. The mixture was allowed to cool to 80° C. and then poured into rapidly stirring heptane (200 mL) and stirred at rt for 18 h. The solids were collected by filtration, washed with heptane and dried in vacuo to afford Example 700B (3.2 g, 19.61 mmol, 75%) that was used in the subsequent step without further purification. LRMS calculated for C₉H₉NO₂: 163; found: 164 (M+H).

### Example 700C 4-(benzyloxy)-7,8-dihydroquinolin-5 (6H)-one

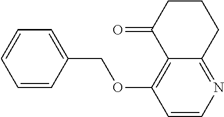 [SMILES: O=C1CCCc2nccc(OCc3ccccc3)c21]

A mixture of Example 700B (300 mg, 1.84 mmol, 1 eq), BnOH (0.57 mL, 5.52 mmol, 3 eq) and PPh₃ (1.45 g, 5.52 mmol, 3 eq) in THE (10 mL) was heated at 50° C. under N₂. DTBAD (1.27 g, 5.52 mmol, 3 eq) was added portionwise, and the mixture was heated for 1.5 h. The mixture was partitioned between DCM and sat. aq. NaHCO₃ solution and the layers were separated. The organic phase was dried (PTFE phase separator) and concentrated in vacuo. The residue was loaded on to a pre-wetted (MeOH) SCX-2 cartridge (10 g), washed with DCM, MeOH and eluting with DCM/7 M NH₃ solution in MeOH (4:1). Further purification by automated flash chromatography (Combiflash Rf, Silica 12 g RediSep™ cartridge) eluting with a gradient of 0-100% EtOAc in hexane afforded Example 700C, isolated as a yellow gum (350 mg, 1.38 mmol, 75%). LRMS calculated for C₁₆H₁₅NO₂: 253; found: 254 (M+H). ¹H NMR (400 MHz, DMSO-d₆) δ ppm: 8.46 (d, J=5.9 Hz, 1H), 7.57-7.52 (m, 2H), 7.45-7.38 (m, 2H), 7.37-7.31 (m, 1H), 7.13 (d, J=5.9 Hz, 1H), 5.29 (s, 2H), 3.01 (t, J=6.2 Hz, 2H), 2.61-2.55 (m, 2H), 2.06-1.97 (m, 2H).

### Example 700D 4-(benzyloxy)-6,6-difluoro-7,8-dihydroquinolin-5 (6H)-one

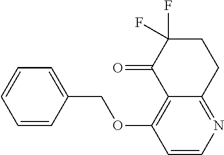 [SMILES: O=C1c2c(OCc3ccccc3)ccnc2CCC1(F)F]

LiHMDS (17.63 mL, 1 M in THF, 17.63 mmol, 2.2 eq) was added dropwise to a solution of Example 700C (2.03 g, 8.01 mmol, 1 eq) and bis(phenylsulphonyl)(fluoro)amine (5.81 g, 18.43 mmol, 2.3 eq) in THE (75 mL) cooled to −78° C., under N₂. The mixture was stirred for 30 min at −78° C. and then slowly allowed to warm to rt and stirred for 18 h. A mixture of mono and bis fluorinated products was observed. The mixture was filtered through celite, washed with THF, and the filtrate was quenched with sat. aq. NH₄Cl solution. Sat. aq. NaHCO₃ solution was added, and the mixture was extracted with EtOAc. The combined organic extracts were dried (MgSO₄) and concentrated in vacuo. Purification by automated flash chromatography (Combiflash Rf, Silica 80 g Gold RediSep™ cartridge) eluting with a gradient of 0-100% EtOAc in hexane afforded the bis fluorinated compound, Example 700D, isolated as an orange gum (1.95 g, 6.74 mmol, 84%). LRMS calculated for C₁₆H₁₃F₂NO₂: 289; found: 290 (M+H). ¹H NMR (400 MHz, DMSO-d₆) δ ppm: 8.61 (d, J=5.9 Hz, 1H), 7.58-7.53 (m, 2H), 7.48-7.33 (m, 3H), 7.27 (d, J=5.9 Hz, 1H), 5.38 (s, 2H), 3.20 (t, J=6.5 Hz, 2H), 2.75-2.60 (m, 2H).

### Example 700E 4-(benzyloxy)-6,6-difluoro-5-methylidene-5,6,7,8-tetrahydroquinoline

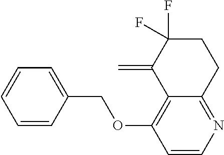 [SMILES: C=C1c2c(OCc3ccccc3)ccnc2CCC1(F)F]

A suspension of methyltriphenylphosphonium bromide (3.24 g, 9.07 mmol, 1.5 eq) in THE (30 mL) was cooled to 0° C. under N₂. LiHMDS (9.07 mL, 1 M in THF, 9.07 mmol, 1.5 eq) was added dropwise, and the mixture was stirred at 0° C. for 30 min. A solution of Example 700D (1.75 g, 6.05 mmol, 1 eq) in THE (8 mL) was added over 2 min and the mixture was allowed to warm to rt and stirred for 18 h. The mixture was cooled to 0° C., quenched by the careful addition of sat. aq. NaHCO₃ solution, and then partitioned with EtOAc. The phases were separated, and the aq. phase was extracted with EtOAc. The combined organic extracts were washed with brine, dried (MgSO₄) and concentrated in vacuo. Purification by automated flash chromatography (Combiflash Rf, Silica 40 g RediSep™ cartridge) eluting with a gradient of 0-87% EtOAc in hexane afforded Example 700E as an orange oil (652 mg, 2.27 mmol, 38%). LRMS calculated for C₁₇H₁₅F₂NO: 287; found: 288 (M+H). ¹H NMR (400 MHz, DMSO-d₆) δ ppm: 8.32 (d, J=5.8 Hz, 1H), 7.52-7.47 (m, 2H), 7.47-7.41 (m, 2H), 7.41-7.35 (m, 1H), 7.16 (d, J=5.8 Hz, 1H), 6.46-6.42 (m, 1H), 5.95-5.91 (m, 1H), 5.31 (s, 2H), 3.07 (t, J=6.9 Hz, 2H), 2.44-2.31 (m, 2H).

### Example 700F 6,6-difluoro-5-methyl-5,6,7,8-tetrahydroquinolin-4-ol

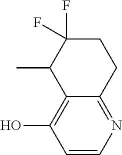

To a solution of Example 700E (652 mg, 2.27 mmol, 1 eq) in EtOAc (30 mL) under a N₂ atmosphere was added Pd/C (30 mg). The mixture was evacuated and back filled with N₂ (×3) and then evacuated and flushed with H₂. The mixture was shaken under a H₂ atmosphere at rt for 7 h. The mixture was filtered through celite, washed with EtOAc and 20% MeOH in DCM and the solvents were removed in vacuo. Purification by reverse phase automated flash chromatography (Combiflash Rf, C18 50 g Gold RediSep™ cartridge) eluting with a gradient of 10-55% MeCN in water afforded a racemate, Example 700F, isolated as a white powder (350 mg, 1.76 mmol, 77%). LRMS calculated for C₁₀H₁₁F₂NO: 199; found: 200 (M+H). ¹H NMR (400 MHz, DMSO-d₆) δ ppm: 11.39 (br s, 1H), 7.58 (d, J=7.2 Hz, 1H), 6.03 (d, J=7.2 Hz, 1H), 3.20-3.07 (m, 1H), 2.90-2.67 (m, 2H), 2.43-2.21 (m, 1H), 2.21-2.07 (m, 1H), 1.13 (dd, J=6.9, 1.5 Hz, 3H).

### Example 700 (1r,2′S,4S)-4-(3-chloroanilino)-2′-[(2R)-3-{[(5S)-6,6-difluoro-5-methyl-5,6,7,8-tetrahydroquinolin-4-yl]oxy}-2-methylpropyl]-2′,3′-dihydrospiro[cyclohexane-1,1′-indene]-4-carboxylic acid

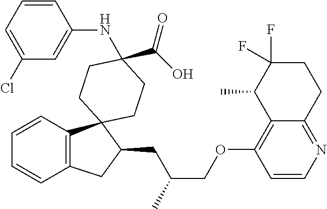 [SMILES: C[C@@H](COc1ccnc2c1[C@H](C)C(F)(F)CC2)C[C@H]1Cc2ccccc2[C@]12CC[C@@](Nc1cccc(Cl)c1)(C(=O)O)CC2]

and

### Example 701 (1r,2′S,4S)-4-(3-chloroanilino)-2′-[(2R)-3-{[(5R)-6,6-difluoro-5-methyl-5,6,7,8-tetrahydroquinolin-4-yl]oxy}-2-methylpropyl]-2′,3′-dihydrospiro[cyclohexane-1,1′-indene]-4-carboxylic acid

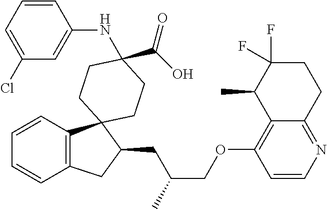 [SMILES: C[C@@H](COc1ccnc2c1[C@@H](C)C(F)(F)CC2)C[C@H]1Cc2ccccc2[C@]12CC[C@@](Nc1cccc(Cl)c1)(C(=O)O)CC2]

Using General procedure 30a and Preparation 10b1 as the appropriate indane and Example 700F as the appropriate alcohol, a mixture of diastereoisomers was obtained. The diastereoisomers were separated by chiral chromatography. Column: AD, 50×500 mm, 20 μm, Eluents: EtOH/heptane. The diastereoisomer eluting earlier was hydrolyzed according to General Procedure 33a to obtain Example 700. ¹H NMR (500 MHz, DMSO-d₆) δ ppm: 12.69 (br s, 1H), 8.26 (d, 1H), 7.39-7.10 (m, 4H), 7.03 (t, 1H), 6.89 (d, 1H), 6.60 (t, 1H), 6.55 (dm, 1H), 6.53 (dm, 1H), 6.25 (br s, 1H), 3.93/3.91 (dd+dd, 2H), 3.28 (m, 1H), 3.03/2.90 (m+m, 2H), 3.02/2.55 (dd+dd, 2H), 2.50-1.30 (m, 10H), 2.16 (m, 1H), 2.02 (m, 1H), 1.48/1.31 (m+m, 2H), 1.10 (d, 3H), 1.05 (d, 3H). HRMS calculated for C₃₅H₃₉N₂O₃F₂Cl: 608.2617; found: 609.2676 (M+H).

The diastereoisomer eluting later was hydrolyzed according to General procedure 33a to obtain Example 701. ¹H NMR (500 MHz, DMSO-d₆) δ ppm: 12.68 (br s, 1H), 8.26 (d, 1H), 7.39-7.10 (m, 4H), 7.04 (t, 1H), 6.88 (d, 1H), 6.62 (t, 1H), 6.53 (m, 2H), 6.24 (br s, 1H), 3.96/3.91 (dd+dd, 2H), 3.26 (m, 1H), 3.04/2.90 (m+m, 2H), 3.02/2.55 (dd+dd, 2H), 2.51-1.35 (m, 10H), 2.16 (m, 1H), 2.02 (m, 1H), 1.40/1.36 (m+m, 2H), 1.23 (d, 3H), 1.06 (d, 3H). HRMS calculated for C₃₅H₃₉N₂O₃F₂Cl: 608.2617; found: 609.2690 (M+H).

### Example 702

### Example 702A 2″-{(2R)-3-[(4-methoxyphenyl)methoxy]-2-methylpropyl}dispiro[[1,3]dioxolane-2,1′-cyclohexane-4′,1″-indene]

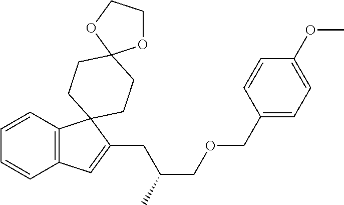 [SMILES: COc1ccc(COC[C@H](C)CC2=Cc3ccccc3C23CCC2(CC3)OCCO2)cc1]

Using General Procedure 27A and Preparation 4aA as the 2-bromo-indene derivative and Preparation 3d as the appropriate Zn reagent, Example 702A was isolated as a white solid. ¹H NMR (400 MHz, CDCl₃) δ ppm: 7.72-7.67 (m, 1H), 7.31-7.22 (m, 4H), 7.12-7.06 (m, 1H), 6.91-6.86 (m, 2H), 6.43-6.39 (m, 1H), 4.49-4.42 (m, 2H), 4.07-4.01 (m, 4H), 3.81 (s, 3H), 3.41 (dd, J=9.1, 5.8 Hz, 1H), 3.33 (dd, J=9.1, 6.5 Hz, 1H), 2.43 (ddd, J=16.5, 5.7, 1.8 Hz, 1H), 2.33-2.20 (m, 3H), 2.17-1.97 (m, 3H), 1.91-1.82 (m, 2H), 1.24-1.15 (m, 2H), 1.02 (d, J=6.6 Hz, 3H).

### Example 702B 2″-{(2R)-3-[(4-methoxyphenyl)methoxy]-2-methylpropyl}-2″,3″-dihydrodispiro[[1,3]dioxolane-2,1′-cyclohexane-4′,1″-indene]

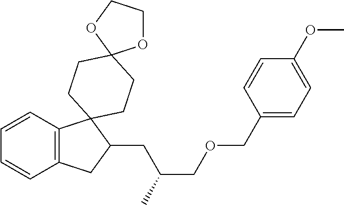 [SMILES: COc1ccc(COC[C@H](C)CC2Cc3ccccc3C23CCC2(CC3)OCCO2)cc1]

and

### Example 702C (2″R)-2″-{(2R)-3-[(4-methoxyphenyl)methoxy]-2-methylpropyl}-2″,3″-dihydrodispiro[[1,3]dioxolane-2,1′-cyclohexane-4′,1″-indene]

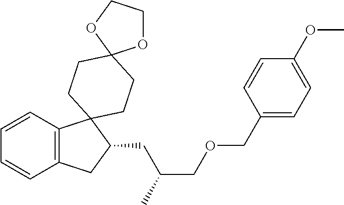 [SMILES: COc1ccc(COC[C@H](C)C[C@@H]2Cc3ccccc3C23CCC2(CC3)OCCO2)cc1]

To a solution of Example 702A (40 g, 92.1 mmol, 1 eq) in EtOAc (100 mL) and MeOH (300 mL) was added 10% Pd/C (2.00 g) under a N₂ atmosphere. The mixture was evacuated and backfilled with N₂ (×3), then evacuated and backfilled with H₂ and stirred at 50° C. for 48 h under an atmosphere of H₂. The mixture was filtered through a large plug of celite and the filtrate was concentrated under reduced pressure to afford Example 702B as a 2:1 mixture of diastereoisomers, isolated as a yellow oil (27.4 g, 62.8 mmol, 68%). ¹H NMR (400 MHz, CDCl₃) δ ppm: 7.36-7.12 (m, 6H), 6.92-6.83 (m, 2H), 4.50-4.37 (m, 2H), 4.04-3.95 (m, 4H), 3.81/3.80 (s, 3H), 3.47-3.18 (m, 2H), 3.07-2.96 (m, 1H), 2.65-2.55 (m, 1H), 2.36-2.25 (m, 1H), 2.03-1.56 (m, 9H), 1.51-1.42/1.38-1.28 (m, 1H), 1.18-1.02 (m, 1H), 1.01-0.93 (m, 3H).

The plug of celite was eluted with a 75% solution of EtOAC in MeOH until no further material could be detected. The filtrate was concentrated in vacuo to afford a single diastereoisomer, Example 702C, isolated as a white solid (11.83 g, 27.1 mmol, 29%). ¹H NMR (400 MHz, CDCl₃) δ ppm: 7.35-7.25 (m, 3H), 7.22-7.12 (m, 3H), 6.91-6.85 (m, 2H), 4.46 (d, J=11.6 Hz, 1H), 4.41 (d, J=11.6 Hz, 1H), 4.00 (s, 4H), 3.80 (s, 3H), 3.43 (dd, J=9.0, 4.5 Hz, 1H), 3.25 (dd, J=9.0, 6.7 Hz, 1H), 3.02 (dd, J=15.7, 7.0 Hz, 1H), 2.60 (dd, J=15.7, 5.3 Hz, 1H), 2.34-2.24 (m, 1H), 2.01-1.91 (m, 1H), 1.90-1.55 (m, 8H), 1.50-1.41 (m, 1H), 1.12-1.02 (m, 1H), 0.98 (d, J=6.8 Hz, 3H).

### Example 702D (2′S)-2′-{(2R)-3-[(4-methoxyphenyl)methoxy]-2-methylpropyl}-2′,3′-dihydrospiro[cyclohexane-1,1′-inden]-4-one

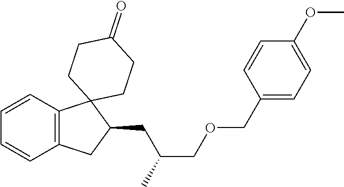 [SMILES: COc1ccc(COC[C@H](C)C[C@H]2Cc3ccccc3C23CCC(=O)CC3)cc1]

To a solution of Example 702B (16.4 g, 37.6 mmol, 1 eq) in a mixture of acetone (150 mL) and water (150 mL) was added PPTS (18.9 g, 75.1 mmol, 2 eq) and the mixture was heated at 60° C. for 24 h. The mixture was allowed to cool to rt and concentrated in vacuo. The residue was diluted with water and the pH was adjusted to 8 with 1 M aq. NaOH solution and extracted with EtOAc. The combined organic extracts were washed with brine, dried (MgSO₄) and concentrated in vacuo. The diastereoisomers were purified and separated by automated flash chromatography (CombiFlash Rf, 330 g Gold RediSep™ silica cartridge) eluting with a gradient of 0-20% EtOAc in heptane. The diastereoisomer eluting later was collected as Example 702D, isolated as a colourless oil (6.64 g, 16.9 mmol, 45%)¹H NMR (400 MHz, CDCl₃) S ppm: 7.32-7.16 (m, 6H), 6.90-6.84 (m, 2H), 4.43 (s, 2H), 3.80 (s, 3H), 3.26 (d, J=6.4 Hz, 2H), 3.06 (dd, J=15.7, 7.1 Hz, 1H), 2.75-2.46 (m, 4H), 2.45-2.31 (m, 2H), 2.12-1.81 (m, 5H), 1.54-1.44 (m, 1H), 1.22-1.13 (m, 1H), 0.96 (d, J=6.6 Hz, 3H). HRMS calculated for C₂₆H₃₂O₃: 392.2351; found 410.2683 (M+NH₄).

### Example 702E (2′S)-2′-[(2R)-3-hydroxy-2-methylpropyl]-2′,3′-dihydrospiro[cyclohexane-1,1′-inden]-4-one

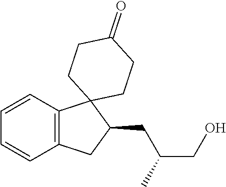 [SMILES: C[C@@H](CO)C[C@H]1Cc2ccccc2C12CCC(=O)CC2]

To a solution of Example 702D (7.14 g, 18.2 mmol, 1 eq) in MeCN (150 mL) was added tBuBr (2.45 mL, 21.8 mmol, 1.2 eq) and the mixture was heated at 90° C. for 3 h. The mixture was allowed to cool to rt and the solvent was removed in vacuo. Purification by automated flash chromatography (CombiFlash Rf, 120 g RediSep™ silica cartridge) eluting with a gradient of 10-65% EtOAc in heptane afforded a single diastereoisomer, Example 702E, isolated as a yellow gum (3.2 g, 11.8 mmol, 65%). ¹H NMR (400 MHz, DMSO-d₆) δ ppm: 7.39-7.32 (m, 1H), 7.26-7.20 (m, 1H), 7.19-7.12 (m, 2H), 4.45 (t, J=5.4 Hz, 1H), 3.27-3.15 (m, 2H), 3.04 (dd, J=15.7, 6.9 Hz, 1H), 2.64-2.49 (m, 3H), 2.46-2.25 (m, 3H), 2.02-1.86 (m, 3H), 1.83-1.73 (m, 1H), 1.66-1.53 (m, 1H), 1.39-1.29 (m, 1H), 1.14-1.04 (m, 1H), 0.89 (d, J=6.6 Hz, 3H).

### Example 702F (2R)-3-[(2″S)-2″,3″-dihydrodispiro[[1,3]dioxolane-2,1′-cyclohexane-4′,1″-inden]-2″-yl]-2-methylpropan-1-ol

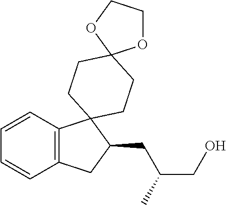 [SMILES: C[C@@H](CO)C[C@H]1Cc2ccccc2C12CCC1(CC2)OCCO1]

To a solution of Example 702E (3.2 g, 11.8 mmol, 1 eq) in toluene (90 mL) was added ethylene glycol (6.57 mL, 118 mmol, 10 eq) followed by PTSA (224 mg, 1.17 mmol, 0.1 eq) and the mixture was heated at 125° C. for 48 h. The mixture was allowed to cool to rt, filtered through celite, washed with EtOAc and the filtrate was concentrated in vacuo. Purification by automated flash chromatography (CombiFlash Rf, 80 g RediSep™ silica cartridge) eluting with a gradient of 0-48% EtOAc in heptane afforded Example 702F as a colourless gum (2.74 g, 8.66 mmol, 74%). ¹H NMR (400 MHz, DMSO-d₆) δ ppm: 7.28-7.09 (m, 4H), 4.44 (t, J=5.4 Hz, 1H), 3.94-3.86 (m, 4H), 3.26-3.13 (m, 2H), 2.98 (dd, J=15.7, 6.9 Hz, 1H), 2.56-2.49 (m, 1H), 2.31-2.20 (m, 1H), 1.90-1.63 (m, 6H), 1.62-1.43 (m, 3H), 1.27-1.18 (m, 1H), 1.03-0.93 (m, 1H), 0.87 (d, J=6.6 Hz, 3H).

### Example 702G (5R)-4-{(2R)-3-[(2″S)-2″,3″-dihydrodispiro[[1,3]dioxolane-2,1′-cyclohexane-4′,1″-inden]-2″-yl]-2-methylpropoxy}-5-methyl-5,6,7,8-tetrahydroquinoline

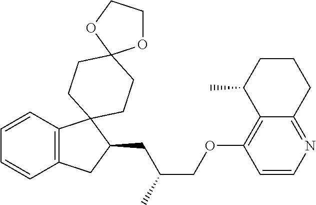 [SMILES: C[C@@H](COc1ccnc2c1[C@H](C)CCC2)C[C@H]1Cc2ccccc2C12CCC1(CC2)OCCO1]

Using General procedure 30a and Example 702F as the appropriate indane and Preparation 2a1 as the appropriate alcohol, a single diastereoisomer, Example 702G was isolated as a colourless gum. LRMS calculated for C₃₀H₃₉NO₃: 461; found: 462 (M+H).

### Example 702H (2′S)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-inden]-4-one

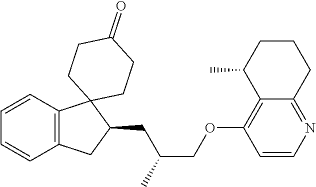 [SMILES: C[C@@H](COc1ccnc2c1[C@H](C)CCC2)C[C@H]1Cc2ccccc2C12CCC(=O)CC2]

Using General procedure 9 and Example 702G as the appropriate ketal, a single diastereoisomer, Example 702H, was isolated as a cream gum. LRMS calculated for C₂₈H₃₅NO₂: 417; found: 418 (M+H). ¹H NMR (400 MHz, DMSO-d₆) δ ppm: 8.15 (d, J=5.6 Hz, 1H), 7.38-7.32 (m, 1H), 7.26-7.20 (m, 1H), 7.20-7.10 (m, 2H), 6.76 (d, J=5.6 Hz, 1H), 3.86 (d, J=6.3 Hz, 2H), 3.10 (dd, J=15.7, 6.9 Hz, 1H), 3.06-2.97 (m, 1H), 2.81-2.72 (m, 1H), 2.72-2.57 (m, 3H), 2.57-2.46 (m, 2H), 2.40-2.26 (m, 2H), 2.10-1.56 (m, 9H), 1.55-1.45 (m, 1H), 1.39-1.29 (m, 1H), 1.08 (d, J=6.7 Hz, 3H), 1.01 (d, J=6.8 Hz, 3H).

### Example 702 (1r,2′S,4S)-4-(3-chloro-4-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

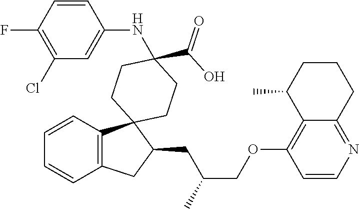 [SMILES: C[C@@H](COc1ccnc2c1[C@H](C)CCC2)C[C@H]1Cc2ccccc2[C@]12CC[C@@](Nc1ccc(F)c(Cl)c1)(C(=O)O)CC2]

Using General procedure 10 and Example 702H as the appropriate ketone and 3-chloro-4-fluoroaniline as the appropriate aniline, a single diastereoisomer, Example 702 was isolated as a white powder. LRMS calculated for C₃₅H₄₀ClFN₂O₃: 590; found: 591 (M+H). ¹H NMR (400 MHz, DMSO-d₆) δ ppm: 8.15 (d, J=5.6 Hz, 1H), 7.41-7.33 (m, 1H), 7.24-7.06 (m, 4H), 6.78 (d, J=5.6 Hz, 1H), 6.69 (dd, J=6.3, 2.8 Hz, 1H), 6.58-6.51 (m, 1H), 3.95-3.83 (m, 2H), 3.10-2.96 (m, 2H), 2.82-2.72 (m, 1H), 2.72-2.60 (m, 1H), 2.59-2.38 (m, 2H), 2.21-2.07 (m, 2H), 2.07-1.56 (m, 9H), 1.56-1.44 (m, 2H), 1.44-1.29 (m, 2H), 1.09-1.01 (m, 6H).

### Example 703

### Example 703A (2′R)-2′-[(2R)-3-hydroxy-2-methylpropyl]-2′,3′-dihydrospiro[cyclohexane-1,1′-inden]-4-one

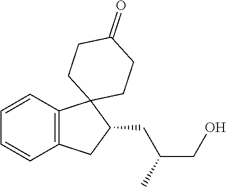 [SMILES: C[C@@H](CO)C[C@@H]1Cc2ccccc2C12CCC(=O)CC2]

To a solution of Example 702C (2.6 g, 5.96 mmol, 1 eq) in MeCN (50 mL) was added tBuBr (0.8 mL, 7.15 mmol, 1.2 eq) and the mixture was heated at 85° C. for 3.5 h. The mixture was allowed to cool to rt and the solvent was removed in vacuo. Purification by automated flash chromatography (CombiFlash Rf, 40 g RediSep™ silica cartridge) eluting with a gradient of 0-65% EtOAc in heptane afforded Example 703A as a colourless gum (1.21 g, 4.43 mmol, 74%). LRMS calculated for C₁₈H₂₄O₂: 272; found: 273 (M+H). ¹H NMR (400 MHz, DMSO-d₆) δ ppm: 7.38-7.31 (m, 1H), 7.27-7.20 (m, 1H), 7.20-7.10 (m, 2H), 4.44 (t, J=5.1 Hz, 1H), 3.45-3.37 (m, 1H), 3.37-3.28 (m, 1H), 3.07 (dd, J=15.7, 6.9 Hz, 1H), 2.67-2.27 (m, 6H), 2.01-1.89 (m, 3H), 1.81-1.47 (m, 3H), 1.01-0.86 (m, 4H).

### Example 703B (2R)-3-[(2″R)-2″,3″-dihydrodispiro[[1,3]dioxolane-2,1′-cyclohexane-4′,1″-inden]-2″-yl]-2-methylpropan-1-ol

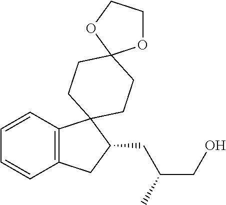 [SMILES: C[C@@H](CO)C[C@@H]1Cc2ccccc2C12CCC1(CC2)OCCO1]

To a solution of Example 703A (1.2 g, 4.41 mmol, 1 eq) in toluene (30 mL) was added ethylene glycol (2.46 mL, 44.1 mmol, 10 eq) followed by PTSA (0.04 mL, 0.44 mmol, 0.1 eq) and the mixture was heated at 125° C. for 48 h. The mixture was allowed to cool to rt, filtered through celite, washed with EtOAc and the filtrate was concentrated in vacuo. Purification by automated flash chromatography (CombiFlash Rf, 24 g RediSep™ silica cartridge) eluting with a gradient of 0-60% EtOAc in heptane afforded Example 703B as a colourless oil (1.35 g, 4.27 mmol, 87%). ¹H NMR (400 MHz, DMSO-d₆) δ ppm: 7.28-7.08 (m, 4H), 4.43 (t, J=5.0 Hz, 1H), 3.94-3.86 (m, 4H), 3.45-3.38 (m, 1H), 3.29-3.22 (m, 1H), 3.01 (dd, J=15.8, 6.9 Hz, 1H), 2.56 (dd, J=15.8, 4.9 Hz, 1H), 2.33-2.22 (m, 1H), 1.89-1.51 (m, 8H), 1.51-1.35 (m, 2H), 0.92-0.80 (m, 4H).

### Example 703C (5R)-4-{(2R)-3-[(2″R)-2″,3″-dihydrodispiro[[1,3]dioxolane-2,1′-cyclohexane-4′,1″-inden]-2″-yl]-2-methylpropoxy}-5-methyl-5,6,7,8-tetrahydroquinoline

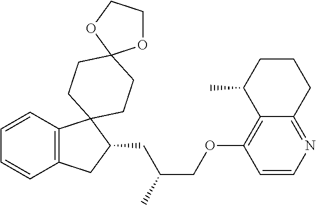 [SMILES: C[C@@H](COc1ccnc2c1[C@H](C)CCC2)C[C@@H]1Cc2ccccc2C12CCC1(CC2)OCCO1]

Using General procedure 30a and Example 703B as the appropriate indane and Preparation 2a1 as the appropriate alcohol, Example 703C was isolated as a colourless gum. LRMS calculated for C₃₀H₃₉NO₃: 461; found: 462 (M+H).

### Example 703D (2′R)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-inden]-4-one

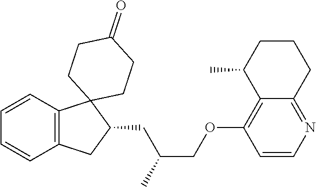 [SMILES: C[C@@H](COc1ccnc2c1[C@H](C)CCC2)C[C@@H]1Cc2ccccc2C12CCC(=O)CC2]

Using General procedure 9 and Example 703C as the appropriate ketal, Example 703D was isolated as a colourless oil. LRMS calculated for C₂₈H₃₅NO₂: 417; found: 418 (M+H).

### Example 703 (1r,2′R,4R)-4-(3-chloro-4-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

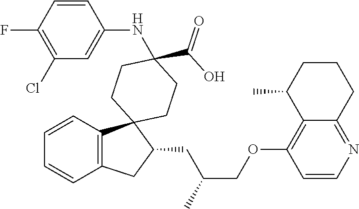 [SMILES: C[C@@H](COc1ccnc2c1[C@H](C)CCC2)C[C@@H]1Cc2ccccc2[C@]12CC[C@@](Nc1ccc(F)c(Cl)c1)(C(=O)O)CC2]

Using General procedure 10 and Example 703D as the appropriate ketone and 3-chloro-4-fluoroaniline as the appropriate aniline, a single diastereoisomer, Example 703 was isolated as a cream solid. LRMS calculated for C₃₅H₄₀ClFN₂O₃: 590; found: 591 (M+H). ¹H NMR (400 MHz, DMSO-d₆) δ ppm: 12.76 (br s, 1H), 8.14 (d, J=5.6 Hz, 1H), 7.45-7.34 (m, 1H), 7.24-7.05 (m, 4H), 6.79 (d, J=5.6 Hz, 1H), 6.67 (dd, J=6.3, 2.8 Hz, 1H), 6.55-6.47 (m, 1H), 6.06 (br s, 1H), 4.00 (dd, J=9.6, 4.5 Hz, 1H), 3.89 (dd, J=9.6, 5.2 Hz, 1H), 3.06-2.94 (m, 2H), 2.80-2.69 (m, 1H), 2.67-2.54 (m, 2H), 2.46-2.34 (m, 1H), 2.17-1.93 (m, 4H), 1.92-1.59 (m, 6H), 1.58-1.19 (m, 5H), 1.16-1.04 (m, 6H).

### Example 704

### Example 704A (2′S)-4-(3-chloro-5-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carbonitrile

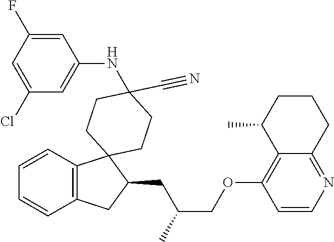 [SMILES: C[C@@H](COc1ccnc2c1[C@H](C)CCC2)C[C@H]1Cc2ccccc2C12CCC(C#N)(Nc1cc(F)cc(Cl)c1)CC2]

Using General Procedure 11 and Example 702H as the appropriate ketone and 3-chloro-5-fluoroaniline as the appropriate aniline, a mixture of diastereoisomers, Example 704A was obtained as a colourless glass. LRMS calculated for C₃₅H₃₉ClFN₃O: 571; found: 572 (M+H).

### Example 704B (1r,2′S,4S)-4-(3-chloro-5-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxamide

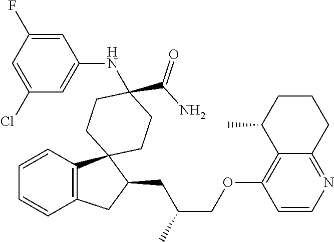 [SMILES: C[C@@H](COc1ccnc2c1[C@H](C)CCC2)C[C@H]1Cc2ccccc2[C@]12CC[C@@](Nc1cc(F)cc(Cl)c1)(C(N)=O)CC2]

Using General Procedure 12a and Example 704A as the appropriate nitrile, a mixture of diastereoisomers was obtained. The diastereoisomers were purified and separated by automated flash chromatography (Combiflash Rf, Silica 12 g Gold RediSep column) eluting with a gradient of 0-10% MeOH in EtOAc. The diastereoisomer eluting earlier was collected as Example 704B, isolated as a colourless glass. LRMS calculated for C₃₅H₄₁ClFN₃O₂: 589; found: 590 (M+H).

### Example 704 (1r,2′S,4S)-4-(3-chloro-5-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

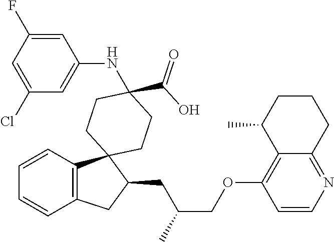 [SMILES: C[C@@H](COc1ccnc2c1[C@H](C)CCC2)C[C@H]1Cc2ccccc2[C@]12CC[C@@](Nc1cc(F)cc(Cl)c1)(C(=O)O)CC2]

Using General Procedure 13 and Example 704B as the appropriate amide, Example 704 was isolated as a white powder. LRMS calculated for C₃₅H₄₀ClFN₂O₃: 590; found: 591 (M+H). ¹H NMR (400 MHz, DMSO-d₆) δ ppm: 8.15 (d, J=5.7 Hz, 1H), 7.41-7.34 (m, 1H), 7.24-7.12 (m, 3H), 6.78 (d, J=5.7 Hz, 1H), 6.61 (br s, 1H), 6.50-6.44 (m, 2H), 6.34-6.27 (m, 1H), 3.95-3.84 (m, 2H), 3.10-2.97 (m, 2H), 2.81-2.72 (m, 1H), 2.72-2.60 (m, 1H), 2.59-2.39 (m, 2H), 2.20-2.07 (m, 2H), 2.06-1.94 (m, 2H), 1.94-1.56 (m, 7H), 1.56-1.45 (m, 2H), 1.45-1.30 (m, 2H), 1.10-1.01 (m, 6H).

### Example 705 (1r,2′S,4S)-4-(5-chloro-2-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

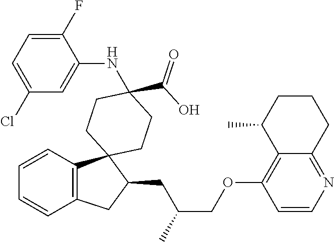 [SMILES: C[C@@H](COc1ccnc2c1[C@H](C)CCC2)C[C@H]1Cc2ccccc2[C@]12CC[C@@](Nc1cc(Cl)ccc1F)(C(=O)O)CC2]

Using General procedure 10 and Example 702H as the appropriate ketone, and 5-chloro-2-fluoroaniline as the appropriate aniline, a single diastereoisomer, Example 705 was isolated as a white powder. LRMS calculated for C₃₅H₄₀ClFN₂O₃: 590; found: 591 (M+H). ¹H NMR (400 MHz, DMSO-d₆) δ ppm: 8.15 (d, J=5.6 Hz, 1H), 7.42-7.32 (m, 1H), 7.24-7.02 (m, 4H), 6.78 (d, J=5.6 Hz, 1H), 6.65-6.56 (m, 2H), 5.65 (br s, 1H), 3.93-3.81 (m, 2H), 3.09-2.95 (m, 2H), 2.82-2.71 (m, 1H), 2.71-2.60 (m, 1H), 2.60-2.38 (m, 2H), 2.23-1.94 (m, 4H), 1.94-1.55 (m, 7H), 1.55-1.38 (m, 3H), 1.38-1.27 (m, 1H), 1.09-0.99 (m, 6H).

### Example 706

### Example 706A di-tert-butyl (1′s,1″s)-2″-bromo-2,5-dioxodispiro[imidazolidine-4,1′-cyclohexane-4′,1″-indene]-1,3-dicarboxylate

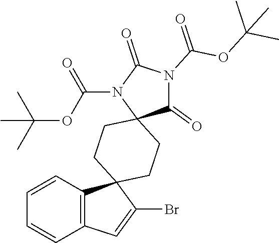 [SMILES: CC(C)(C)OC(=O)N1C(=O)N(C(=O)OC(C)(C)C)[C@]2(CC[C@]3(CC2)C(Br)=Cc2ccccc23)C1=O]

To a solution of Preparation 5aA (17.9 g, 51.7 mmol, 1 eq) and Boc₂0 (44.2 mL, 207 mmol, 4 eq) in THE (300 mL) was added DMAP (316 mg, 2.58 mmol, 0.05 eq) and TEA (7.9 mL, 56.8 mmol, 1.1 eq). The mixture was stirred at rt for 16 h. The mixture was partitioned between EtOAc and water, and the organic phase was washed with brine, dried (MgSO₄) and concentrated in vacuo. Purification by automated flash chromatography (CombiFlash Rf, 330 g RediSep™ silica cartridge) eluting with a gradient of 0-12% EtOAc in heptane afforded Example 706A as a cream solid (24 g, 43.8 mmol, 85%). ¹H NMR (400 MHz, DMSO-d₆) δ ppm: 7.70-7.63 (m, 1H), 7.34-7.20 (m, 3H), 7.00 (s, 1H), 2.99-2.87 (m, 2H), 2.26-2.11 (m, 4H), 1.91-1.82 (m, 2H), 1.55 (s, 9H), 1.53 (s, 9H).

### Example 706B di-tert-butyl (1′R,1″r)-2″-{(2R)-3-[(4-methoxyphenyl)methoxy]-2-methylpropyl}-2,5-dioxodispiro[imidazolidine-4,1′-cyclohexane-4′,1″-indene]-1,3-dicarboxylate

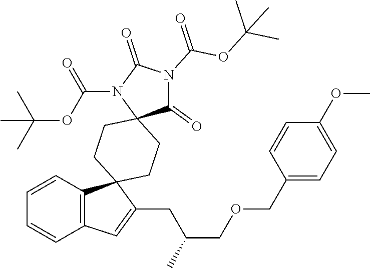 [SMILES: COc1ccc(COC[C@H](C)CC2=Cc3ccccc3[C@]23CC[C@]2(CC3)C(=O)N(C(=O)OC(C)(C)C)C(=O)N2C(=O)OC(C)(C)C)cc1]

Using General Procedure 27b and Example 706A as the appropriate 2-bromo-indene and Preparation 3d as the appropriate Zn reagent, Example 706B was obtained as a white foam. ¹H NMR (400 MHz, DMSO-d₆) δ ppm: 7.53-7.46 (m, 1H), 7.29-7.10 (m, 5H), 6.93-6.85 (m, 2H), 6.40-6.34 (m, 1H), 4.43 (d, J=11.6 Hz, 1H), 4.37 (d, J=11.6 Hz, 1H), 3.73 (s, 3H), 3.43-3.29 (m, 2H), 2.80-2.67 (m, 3H), 2.47-2.38 (m, 1H), 2.30-2.03 (m, 5H), 1.69-1.57 (m, 2H), 1.55 (s, 9H), 1.50 (s, 9H), 1.01 (d, J=6.6 Hz, 3H).

### Example 706C (1r,4R)-4-amino-2′-{(2R)-3-[(4-methoxyphenyl)methoxy]-2-methylpropyl}spiro[cyclohexane-1,1′-indene]-4-carboxylic acid

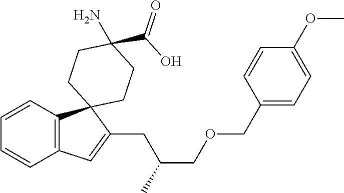 [SMILES: COc1ccc(COC[C@H](C)CC2=Cc3ccccc3[C@]23CC[C@@](N)(C(=O)O)CC3)cc1]

To a solution of Example 706B (19 g, 28.98 mmol, 1 eq) in DME (300 mL) was added 1 M aq. NaOH solution (259 mL, 259 mmol, 9 eq) and the mixture was stirred at rt for 3 h. The mixture was concentrated in vacuo and the aq. phase was neutralized with 2 M aq. HCl solution. The mixture was partitioned between DCM and water, the phases separated, and the organic phase was washed with brine, dried (MgSO₄) and concentrated in vacuo to afford Example 706C as an overweight cream foam (12.5 g). LRMS calculated for C₂₇H₃₃NO₄: 435; found: 436 (M+H).

### Example 706D methyl (1r,4R)-4-amino-2′-[(2R)-3-hydroxy-2-methylpropyl]spiro[cyclohexane-1,1′-indene]-4-carboxylate

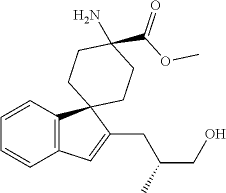 [SMILES: COC(=O)[C@]1(N)CC[C@]2(CC1)C(C[C@@H](C)CO)=Cc1ccccc12]

To a solution of Example 706C (12.5 g) in MeOH (200 mL), cooled to 0° C., was added SOCl₂ (6.53 mL, 89.5 mmol) dropwise. The mixture was heated at 70° C. for 48 h and then cooled to rt, neutralized with 7 M NH₃ solution in MeOH, and concentrated in vacuo. Purification by automated flash chromatography (CombiFlash Rf, 120 g RediSep™ silica cartridge) eluting with a gradient of 0-24% EtOAc in heptane afforded Example 706D as a yellow gum (8.43 g, 25.6 mmol, 88% for 2 steps). LRMS calculated for C₂₀H₂₇NO₃: 329; found: 330 (M+H).

### Example 706E methyl (1r,2′S,4S)-4-amino-2′-[(2R)-3-hydroxy-2-methylpropyl]-2′,3′-dihydrospiro[cyclohexane-1,1′-indene]-4-carboxylate

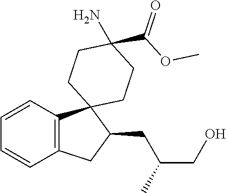 [SMILES: COC(=O)[C@]1(N)CC[C@@]2(CC1)c1ccccc1C[C@@H]2C[C@@H](C)CO]

To a solution of Example 706D (8.40 g, 25.5 mmol, 1 eq) in EtOAc (190 mL) and AcOH (60 mL) was added 10% Pd/C (250 mg) under a N₂ atmosphere. The mixture was evacuated and backfilled with N₂ (×3), then evacuated and backfilled with H₂ and shaken for 24 h at rt under an atmosphere of H₂. A mixture of diastereoisomers was obtained. The reaction was filtered through celite, eluted with EtOAc and concentrated under reduced pressure. Purification by automated flash chromatography (CombiFlash Rf, 330 g RediSep™ silica cartridge) eluting with a gradient of 0-13% MeOH in EtOAc afforded a single diastereoisomer, Example 706E, eluting firstly, isolated as a colourless gum (3.50 g, 10.6 mmol, 41%). LRMS calculated for C₂₀H₂₉NO₃: 331; found: 332 (M+H).

### Example 706F methyl (1r,2′S,4S)-4-amino-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

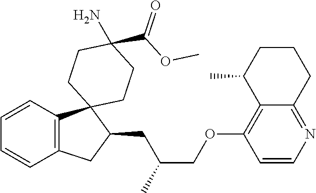 [SMILES: COC(=O)[C@]1(N)CC[C@@]2(CC1)c1ccccc1C[C@@H]2C[C@@H](C)COc1ccnc2c1[C@H](C)CCC2]

Using General procedure 30b and Example 706E as the appropriate indane and Preparation 2a1 as the appropriate alcohol, Example 706F was isolated as a yellow oil. LRMS calculated for C₃₀H₄₀N₂O₃: 476; found: 477 (M+H).

### Example 706G (1r,2′S,4S)-4-amino-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

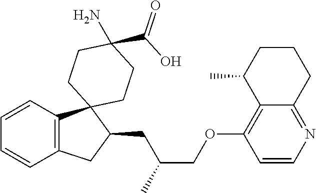 [SMILES: C[C@@H](COc1ccnc2c1[C@H](C)CCC2)C[C@H]1Cc2ccccc2[C@]12CC[C@@](N)(C(=O)O)CC2]

Using General procedure 33b and Example 706F as the appropriate ester, Example 706G was obtained as a white powder (614 mg, 1.33 mmol, 63%). LRMS calculated for C₂₉H₃₈N₂O₃: 462; found: 463 (M+H). ¹H NMR (400 MHz, DMSO-d₆) δ ppm: 8.16 (d, J=5.6 Hz, 1H), 7.54-7.45 (m, 1H), 7.23-7.10 (m, 3H), 6.78 (d, J=5.6 Hz, 1H), 3.91-3.82 (m, 2H), 3.11-2.97 (m, 2H), 2.82-2.73 (m, 1H), 2.73-2.61 (m, 1H), 2.58-2.40 (m, 2H), 2.21-2.09 (m, 2H), 2.06-1.95 (m, 1H), 1.90-1.39 (m, 12H), 1.10-1.03 (m, 6H).

### Example 706 (1r,2′S,4S)-4-(3-chloro-2-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

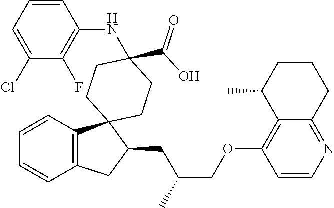 [SMILES: C[C@@H](COc1ccnc2c1[C@H](C)CCC2)C[C@H]1Cc2ccccc2[C@]12CC[C@@](Nc1cccc(Cl)c1F)(C(=O)O)CC2]

Using General procedure 16 and Example 706G as the appropriate amino acid and 1-chloro-2-fluoro-3-iodobenzene as the appropriate iodobenzene and following purification via prep RP-HPLC using 25 mM aq. NH₄HCO₃ solution and MeCN as eluents, Example 706 was obtained. ¹H NMR (500 MHz, DMSO-d₆) δ ppm: 12.81 (br s, 1H), 8.14 (d, 1H), 7.41-7.11 (m, 4H), 6.93 (td, 1H), 6.77 (d, 1H), 6.72 (td, 1H), 6.58 (t, 1H), 5.63 (br, 1H), 3.89/3.86 (dd+dd, 2H), 3.04 (m, 1H), 2.99/2.53 (dd+dd, 2H), 2.76/2.65 (dm+m, 2H), 2.48-1.28 (m, 16H), 1.04 (d, 3H), 1.02 (d, 3H). HRMS calculated for C₃₅H₄₀N₂O₃FCl: 590.2712; found: 591.2767 (M+H).

### Example 707 (1r,2′S,4S)-4-(3-chloro-2,4-difluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

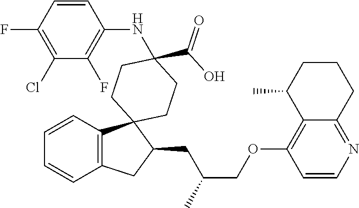 [SMILES: C[C@@H](COc1ccnc2c1[C@H](C)CCC2)C[C@H]1Cc2ccccc2[C@]12CC[C@@](Nc1ccc(F)c(Cl)c1F)(C(=O)O)CC2]

Using General procedure 16 and Example 706G as the appropriate amino acid and 1-bromo-3-chloro-2,4-difluorobenzene as the appropriate bromobenzene, the crude material was obtained. Purification by reverse phase automated flash chromatography at pH 4 (CombiFlash Rf, C18 13 g RediSep column) eluting with a gradient of 10-100% MeCN in water afforded a solid which was dissolved in minimal DCM, then loaded onto a DCM-wet PE-AX cartridge (5 g), washed successively with DCM, MeOH and eluted with 5% HCOOH in DCM, then concentrated in vacuo to afford Example 707 as an off-white powder. LRMS calculated for C₃₅H₃₉ClF₂N₂O₃: 608; found: 609 (M+H). ¹H NMR (400 MHz, DMSO-d₆) δ ppm: 12.83 (br s, 1H), 8.15 (d, J=5.6 Hz, 1H), 7.41-7.34 (m, 1H), 7.25-7.11 (m, 3H), 7.11-7.03 (m, 1H), 6.78 (d, J=5.6 Hz, 1H), 6.69-6.59 (m, 1H), 5.49 (br s, 1H), 3.92-3.84 (m, 2H), 3.08-2.96 (m, 2H), 2.81-2.72 (m, 1H), 2.71-2.60 (m, 1H), 2.59-2.38 (m, 2H), 2.21-1.95 (m, 4H), 1.94-1.27 (m, 11H), 1.05 (d, J=6.7 Hz, 3H), 1.02 (d, J=6.9 Hz, 3H).

### Example 708 (1r,2′S,4S)-4-(3-chloro-2-methylanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

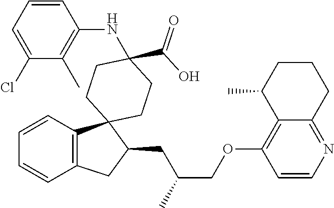 [SMILES: Cc1c(Cl)cccc1N[C@]1(C(=O)O)CC[C@]2(CC1)c1ccccc1C[C@@H]2C[C@@H](C)COc1ccnc2c1[C@H](C)CCC2]

Using General procedure 16 and Example 706G as the appropriate amino acid and 1-chloro-3-iodo-2-methylbenzene as the appropriate iodobenzene and following purification via prep RP-HPLC using 25 mM aq. NH₄HCO₃ solution and MeCN as eluents, Example 708 was obtained. ¹H NMR (500 MHz, DMSO-d₆) δ ppm: 8.15 (d, 1H), 7.46 (d, 1H), 7.21 (dd, 1H), 7.17 (td, 1H), 7.14 (t, 1H), 6.94 (t, 1H), 6.79 (d, 1H), 6.68 (d, 1H), 6.37 (d, 1H), 3.90 (m, 2H), 3.05 (m, 1H), 2.97/2.51 (dd+dd, 2H), 2.77/2.66 (dm+m, 2H), 2.48-1.13 (m, 8H), 2.23 (s, 3H), 2.06 (m, 1H), 2.00 (m, 1H), 1.79/1.74 (m+m, 2H), 1.68/1.60 (m+d, 2H), 1.56/1.37 (m+m, 2H), 1.04 (d, 3H), 1.04 (d, 3H). HRMS calculated for C₃₆H₄₃N₂O₃Cl: 586.2962; found: 587.3034 (M+H).

### Example 709 (1r,2′S,4S)-4-(3-chloro-4-methylanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

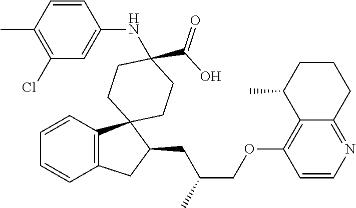 [SMILES: Cc1ccc(N[C@]2(C(=O)O)CC[C@]3(CC2)c2ccccc2C[C@@H]3C[C@@H](C)COc2ccnc3c2[C@H](C)CCC3)cc1Cl]

Using General procedure 10 and Example 702H as the appropriate ketone and 3-chloro-4-methylaniline as the appropriate aniline, a single diastereoisomer, Example 709, was isolated as a white powder. LRMS calculated for C₃₆H₄₃ClN₂O₃: 586; found: 587 (M+H). ¹H NMR (400 MHz, DMSO-d₆) δ ppm: 8.16 (d, J=5.7 Hz, 1H), 7.41-7.34 (m, 1H), 7.24-7.12 (m, 3H), 6.99 (d, J=8.4 Hz, 1H), 6.79 (d, J=5.7 Hz, 1H), 6.65 (d, J=2.4 Hz, 1H), 6.49 (dd, J=8.4, 2.4 Hz, 1H), 3.96-3.83 (m, 2H), 3.10-2.95 (m, 2H), 2.82-2.72 (m, 1H), 2.72-2.60 (m, 1H), 2.59-2.37 (m, 2H), 2.20-1.57 (m, 14H), 1.54-1.42 (m, 2H), 1.42-1.30 (m, 2H), 1.08-1.02 (m, 6H).

### Example 710 (1r,2′S,4S)-4-(3-chloro-5-methylanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

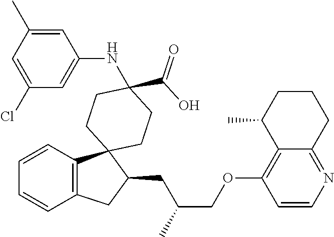 [SMILES: Cc1cc(Cl)cc(N[C@]2(C(=O)O)CC[C@]3(CC2)c2ccccc2C[C@@H]3C[C@@H](C)COc2ccnc3c2[C@H](C)CCC3)c1]

Using General procedure 10 and Example 702H as the appropriate ketone and 3-chloro-5-methylaniline as the appropriate aniline, a single diastereoisomer, Example 710, was isolated as a white powder. LRMS calculated for C₃₆H₄₃ClN₂O₃: 586; found: 587 (M+H). ¹H NMR (400 MHz, DMSO-d₆) δ ppm: 8.16 (d, J=5.7 Hz, 1H), 7.40-7.33 (m, 1H), 7.24-7.12 (m, 3H), 6.79 (d, J=5.7 Hz, 1H), 6.44-6.38 (m, 2H), 6.38-6.35 (m, 1H), 3.95-3.83 (m, 2H), 3.10-2.96 (m, 2H), 2.82-2.72 (m, 1H), 2.71-2.60 (m, 1H), 2.59-2.38 (m, 2H), 2.21-2.07 (m, 5H), 2.07-1.95 (m, 2H), 1.95-1.57 (m, 7H), 1.55-1.28 (m, 4H), 1.10-1.00 (m, 6H).

### Example 711 (1r,2′S,4S)-4-(3-bromoanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

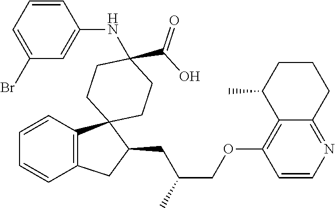 [SMILES: C[C@@H](COc1ccnc2c1[C@H](C)CCC2)C[C@H]1Cc2ccccc2[C@]12CC[C@@](Nc1cccc(Br)c1)(C(=O)O)CC2]

Using General procedure 10 and Example 702H as the appropriate ketone and 3-bromoaniline as the appropriate aniline, a single diastereoisomer, Example 711, was isolated as a beige powder. LRMS calculated for C₃₅H₄₁BrN₂O₃: 616; found: 617 (M+H). ¹H NMR (400 MHz, DMSO-d₆) δ ppm: 12.72 (br s, 1H), 8.15 (d, J=5.5 Hz, 1H), 7.42-7.34 (m, 1H), 7.24-7.11 (m, 3H), 6.99 (t, J=8.0 Hz, 1H), 6.81-6.75 (m, 2H), 6.70-6.65 (m, 1H), 6.61-6.54 (m, 1H), 6.24 (br s, 1H), 3.96-3.82 (m, 2H), 3.11-2.96 (m, 2H), 2.82-2.72 (m, 1H), 2.72-2.60 (m, 1H), 2.59-2.38 (m, 2H), 2.20-2.07 (m, 2H), 2.07-1.95 (m, 2H), 1.95-1.56 (m, 7H), 1.56-1.44 (m, 2H), 1.44-1.30 (m, 2H), 1.09-1.01 (m, 6H).

### Example 712 (1r,2′S,4S)-4-(3-bromo-4-fluoroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

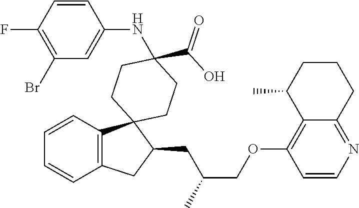 [SMILES: C[C@@H](COc1ccnc2c1[C@H](C)CCC2)C[C@H]1Cc2ccccc2[C@]12CC[C@@](Nc1ccc(F)c(Br)c1)(C(=O)O)CC2]

Using General procedure 10 and Example 702H as the appropriate ketone and 3-bromo-4-fluoroaniline as the appropriate aniline, a single diastereoisomer, Example 712, was isolated as a white powder. LRMS calculated for C₃₅H₄₀BrFN₂O₃: 634; found: 635 (M+H). ¹H NMR (400 MHz, DMSO-d₆) δ ppm: 8.29 (d, J=5.9 Hz, 1H), 7.42-7.33 (m, 1H), 7.24-7.12 (m, 3H), 7.08 (t, J=8.9 Hz, 1H), 6.98 (d, J=5.9 Hz, 1H), 6.84 (dd, J=5.9, 2.8 Hz, 1H), 6.61-6.54 (m, 1H), 4.05-3.92 (m, 2H), 3.11-2.96 (m, 2H), 2.88-2.78 (m, 1H), 2.78-2.66 (m, 1H), 2.59-2.38 (m, 2H), 2.22-1.58 (m, 11H), 1.55-1.30 (m, 4H), 1.09-1.02 (m, 6H).

### Example 713 (1r,2′S,4S)-4-(4-chloro-2,3-dihydro-1H-indol-1-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

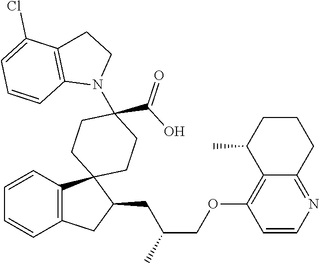 [SMILES: C[C@@H](COc1ccnc2c1[C@H](C)CCC2)C[C@H]1Cc2ccccc2[C@]12CC[C@](C(=O)O)(N1CCc3c(Cl)cccc31)CC2]

Using General procedure 10 and Example 702H as the appropriate ketone and 4-chloroindoline as the appropriate aniline, Example 713 was obtained as a white powder. LRMS calculated for C₃₇H₄₃ClN₂O₃: 598; found: 599 (M+H). ¹H NMR (400 MHz, DMSO-d₆) δ ppm: 8.13 (d, J=5.6 Hz, 1H), 7.28-7.11 (m, 4H), 6.94 (t, J=8.0 Hz, 1H), 6.76 (d, J=5.6 Hz, 1H), 6.58 (d, J=8.0 Hz, 1H), 6.48 (d, J=8.0 Hz, 1H), 3.93-3.79 (m, 2H), 3.76-3.67 (m, 2H), 3.09-2.84 (m, 4H), 2.80-2.71 (m, 1H), 2.70-2.52 (m, 2H), 2.46-2.37 (m, 1H), 2.37-2.19 (m, 2H), 2.13-1.53 (m, 11H), 1.45-1.27 (m, 2H), 1.08 (d, J=6.6 Hz, 3H), 0.99 (d, J=6.9 Hz, 3H).

### Example 714

### Example 714A (1'S,1″r,2″S)-2″-[(2R)-3-hydroxy-2-methylpropyl]-2″,3″-dihydrodispiro[imidazolidine-4,1′-cyclohexane-4′,1″-indene]-2,5-dione

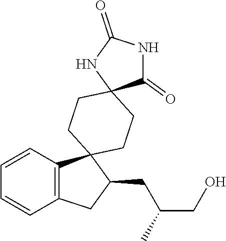 [SMILES: C[C@@H](CO)C[C@H]1Cc2ccccc2[C@]12CC[C@@]1(CC2)NC(=O)NC1=O]

To a solution of the Example 702E (2.65 g, 9.73 mmol, 1 eq) in a mixture of EtOH (40 mL) and water (40 mL) was added NaCN (0.95 g, 19.5 mmol, 2 eq) and (NH₄)₂CO₃ (3.74 g, 38.9 mmol, 4 eq). The mixture was heated at 60° C. for 18 h. A mixture of diastereoisomers was obtained. The mixture was allowed to cool to rt, partitioned between EtOAc and water, and the organic phase was washed with sat. aq. NaHCO₃ solution, brine, dried (MgSO₄) and concentrated in vacuo. Purification by automated flash chromatography (CombiFlash Rf, 80 g Gold RediSep™ silica cartridge) eluting with a gradient of 0-97% EtOAc in heptane afforded a single diastereoisomer, Example 714A, eluting secondly, isolated as a white glass (391 mg, 1.14 mmol, 12%). LRMS calculated for C₂₀H₂₆N₂O₃: 342; found: 343 (M+H).

### Example 714B (1r,2′S,4S)-4-amino-2′-[(2R)-3-hydroxy-2-methylpropyl]-2′,3′-dihydrospiro[cyclohexane-1,1′-indene]-4-carboxylic acid

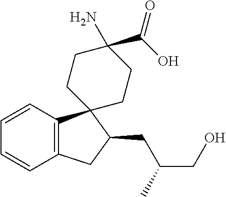 [SMILES: C[C@@H](CO)C[C@H]1Cc2ccccc2[C@]12CC[C@@](N)(C(=O)O)CC2]

Using General procedure 15 and Example 714A as the appropriate hydantoin, Example 714B was obtained as an off-white powder. LRMS calculated for C₁₉H₂₇NO₃: 317; found: 318 (M+H).

### Example 714C methyl (1r,2′S,4S)-4-[(2-chloropyridin-4-yl)amino]-2′-[(2R)-3-hydroxy-2-methylpropyl]-2′,3′-dihydrospiro[cyclohexane-1,1′-indene]-4-carboxylate

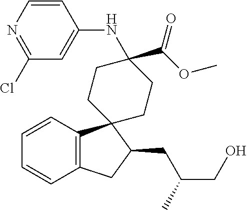 [SMILES: COC(=O)[C@]1(Nc2ccnc(Cl)c2)CC[C@@]2(CC1)c1ccccc1C[C@@H]2C[C@@H](C)CO]

Using General procedure 16 and Example 714B as the appropriate amino acid and 2-chloro-4-iodopyridine as the appropriate iodobenzene, an intermediate was obtained, which was treated according to General procedure 17a to afford Example 714C, isolated as a colourless glass. LRMS calculated for C₂₅H₃₁ClN₂O₃: 442; found: 443 (M+H). ¹H NMR (400 MHz, DMSO-d₆) δ ppm: 7.86 (d, J=5.9 Hz, 1H), 7.41-7.35 (m, 1H), 7.26-7.12 (m, 4H), 6.51-6.42 (m, 2H), 4.47 (t, J=5.3 Hz, 1H), 3.69 (s, 3H), 3.28-3.18 (m, 2H), 2.95 (dd, J=15.6, 7.2 Hz, 1H), 2.56-2.42 (m, 2H), 2.20-1.81 (m, 5H), 1.74-1.44 (m, 3H), 1.43-1.31 (m, 2H), 1.11-1.01 (m, 1H), 0.86 (d, J=6.5 Hz, 3H).

### Example 714 (1r,2′S,4S)-4-[(2-chloropyridin-4-yl)amino]-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

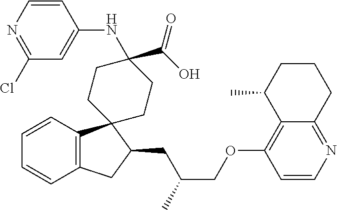 [SMILES: C[C@@H](COc1ccnc2c1[C@H](C)CCC2)C[C@H]1Cc2ccccc2[C@]12CC[C@@](Nc1ccnc(Cl)c1)(C(=O)O)CC2]

Using General procedure 30b and Example 714C as the appropriate indane and Preparation 2a1 as the appropriate alcohol, an intermediate was obtained, which was treated according to General Procedure 33b to obtain Example 714, isolated as a white powder. LRMS calculated for C₃₄H₄₀ClN₃O₃: 573; found: 574 (M+H). ¹H NMR (400 MHz, DMSO-d₆) δ ppm: 8.14 (d, J=5.6 Hz, 1H), 7.65 (d, J=5.8 Hz, 1H), 7.41-7.34 (m, 1H), 7.21-7.08 (m, 3H), 6.77 (d, J=5.6 Hz, 1H), 6.70 (br s, 1H), 6.61-6.54 (m, 2H), 3.94-3.81 (m, 2H), 3.09-2.95 (m, 2H), 2.81-2.72 (m, 1H), 2.72-2.59 (m, 1H), 2.58-2.38 (m, 2H), 2.23-2.10 (m, 2H), 2.06-1.93 (m, 1H), 1.89-1.39 (m, 11H), 1.39-1.29 (m, 1H), 1.10-1.00 (m, 6H).

### Example 715 (1r,2′S,4S)-4-(3,4-dichloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

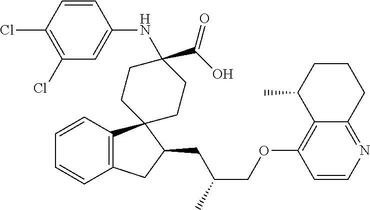 [SMILES: C[C@@H](COc1ccnc2c1[C@H](C)CCC2)C[C@H]1Cc2ccccc2[C@]12CC[C@@](Nc1ccc(Cl)c(Cl)c1)(C(=O)O)CC2]

Using General procedure 16 and Example 706G as the appropriate amino acid and 3,4-dichlorobromobenzene as the appropriate bromobenzene, Example 715 was obtained as a yellow powder. LRMS calculated for C₃₅H₄₀Cl₂N₂O₃: 606; found: 607 (M+H). ¹H NMR (400 MHz, DMSO-d₆) δ ppm: 12.87 (br s, 1H), 8.15 (d, J=5.6 Hz, 1H), 7.42-7.34 (m, 1H), 7.28-7.11 (m, 4H), 6.81-6.75 (m, 2H), 6.57 (dd, J=8.9, 2.7 Hz, 1H), 6.38 (br s, 1H), 3.95-3.82 (m, 2H), 3.10-2.95 (m, 2H), 2.82-2.39 (m, 4H), 2.20-1.29 (m, 15H), 1.08-1.00 (m, 6H).

### Example 716 (1r,2′S,4S)-4-(3-cyanoanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

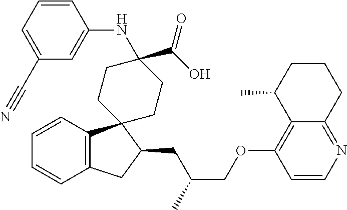 [SMILES: C[C@@H](COc1ccnc2c1[C@H](C)CCC2)C[C@H]1Cc2ccccc2[C@]12CC[C@@](Nc1cccc(C#N)c1)(C(=O)O)CC2]

Using General procedure 10 and Example 702H as the appropriate ketone and 3-aminobenzonitrile as the appropriate aniline, a single diastereoisomer, Example 716 was isolated as a beige solid. LRMS calculated for C₃₆H₄₁N₃O₃: 563; found: 564 (M+H). ¹H NMR (400 MHz, DMSO-d₆) δ ppm: 8.15 (d, J=5.6 Hz, 1H), 7.46-7.32 (m, 1H), 7.29-7.07 (m, 4H), 6.97-6.84 (m, 3H), 6.78 (d, J=5.6 Hz, 1H), 6.48 (br s, 1H), 3.96-3.80 (m, 2H), 3.11-2.94 (m, 2H), 2.82-2.41 (m, 4H), 2.22-1.30 (m, 15H), 1.10-0.99 (m, 6H).

### Example 717

### Example 717A (2′S)-4-(3-chlorophenoxy)-2′-{(2R)-3-[(4-methoxyphenyl)methoxy]-2-methylpropyl}-2′ 3′-dihydrospiro[cyclohexane-1,1′-indene]-4-carboxylic acid

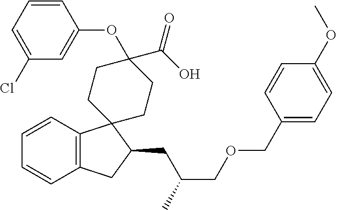 [SMILES: COc1ccc(COC[C@H](C)C[C@H]2Cc3ccccc3C23CCC(Oc2cccc(Cl)c2)(C(=O)O)CC3)cc1]

Using General procedure 10 and Example 702D as the appropriate ketone and 3-chlorophenol as the appropriate phenol, Example 717A was obtained as a mixture of diastereoisomers. ¹H NMR (500 MHz, DMSO-d₆) δ ppm: 7.45-6.76 (m, 12H), 4.39/4.32 (s/s, 2H), 3.73/3.72 (s/s, 3H), 3.26/3.16 (d/m, 2H), 3.02/2.92/2.55/2.48 (dd+dd/dd+dd, 2H), 2.52-0.96 (m, 10H), 2.40/2.01 (m/m, 1H), 1.77 (m, 1H), 0.92/0.89 (d/d, 3H).

### Example 717B methyl (1r,2′S,4S)-4-(3-chlorophenoxy)-2′-{(2R)-3-[(4-methoxyphenyl)methoxy]-2-methylpropyl}-2′,3′-dihydrospiro[cyclohexane-1,1′-indene]-4-carboxylate

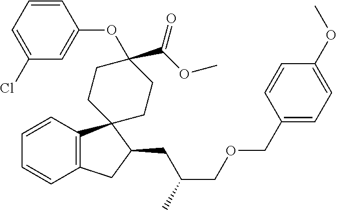 [SMILES: COc1ccc(COC[C@H](C)C[C@H]2Cc3ccccc3[C@]23CC[C@@](Oc2cccc(Cl)c2)(C(=O)OC)CC3)cc1]

Using General procedure 17a and Example 717A as the appropriate amino acid, a mixture of diastereoisomers was obtained. The diastereoisomers were separated via flash chromatography using heptane and EtOAc as eluents. The diastereoisomer eluting earlier was collected as Example 717B. ¹H NMR (500 MHz, DMSO-d₆) δ ppm: 7.44-7.11 (m, 4H), 7.31 (t, 1H), 7.22 (dm, 2H), 7.09 (dm, 1H), 6.88 (t, 1H), 6.88 (dm, 2H), 6.77 (dm, 1H), 4.38 (s, 2H), 3.77 (s, 3H), 3.73 (s, 3H), 3.25 (d, 2H), 2.92/2.14 (dd+dd, 2H), 2.53-1.20 (m, 8H), 2.03 (m, 1H), 1.77 (m, 1H), 1.39/1.17 (td+m, 2H), 0.89 (d, 3H). HRMS calculated for C₃₄H₃₉O₅Cl: 562.2486; found 580.2818 (M+NH₄).

### Example 717C methyl (1r,2′S,4S)-4-(3-chlorophenoxy)-2′-[(2R)-3-hydroxy-2-methylpropyl]-2′,3′-dihydrospiro[cyclohexane-1,1′-indene]-4-carboxylate

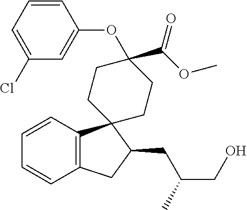 [SMILES: COC(=O)[C@]1(Oc2cccc(Cl)c2)CC[C@@]2(CC1)c1ccccc1C[C@@H]2C[C@@H](C)CO]

Using General procedure 28b and Example 717B as the appropriate PMB derivative, Example 717C was obtained. ¹H NMR (500 MHz, DMSO-d₆) δ ppm: 7.42-7.11 (m, 4H), 7.34 (t, 1H), 7.09 (dm, 1H), 6.89 (t, 1H), 6.79 (dm, 1H), 4.47 (t, 1H), 3.77 (s, 3H), 3.25 (m, 2H), 2.93/2.50 (dd+dd, 2H), 2.50-1.22 (m, 8H), 2.04 (m, 1H), 1.57 (m, 1H), 1.40/1.08 (m+m, 2H), 0.85 (d, 3H). HRMS calculated for C₂₆H₃₁O₄Cl: 442.1911; found 460.2247 (M+NH₄).

### Example 717 (1r,2′S,4S)-4-(3-chlorophenoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

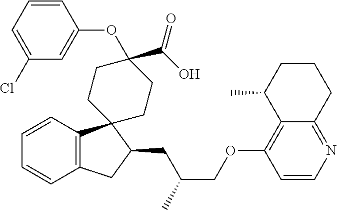 [SMILES: C[C@@H](COc1ccnc2c1[C@H](C)CCC2)C[C@H]1Cc2ccccc2[C@]12CC[C@@](Oc1cccc(Cl)c1)(C(=O)O)CC2]

Using General procedure 32 and Example 717C as the appropriate indane and Preparation 2a1 as the appropriate alcohol, Example 717 was obtained. ¹H NMR (500 MHz, DMSO-d₆) δ ppm: 13.44 (br s, 1H), 8.17 (d, 1H), 7.44-7.12 (m, 4H), 7.28 (t, 1H), 7.04 (dm, 1H), 6.88 (t, 1H), 6.83 (dm, 1H), 6.81 (d, 1H), 3.95/3.91 (dd+dd, 2H), 3.08 (m, 1H), 2.99/2.53 (dd+dd, 2H), 2.77/2.66 (m+m, 2H), 2.53-1.22 (m, 12H), 2.11 (m, 1H), 2.02 (m, 1H), 1.57/1.37 (m+m, 2H), 1.08 (d, 3H), 1.05 (d, 3H). HRMS calculated for C₃₅H₄₀NO₄Cl: 573.2646; found: 574.2712 (M+H).

### Example 718

### Example 718A (2′S)-4-[(3-chlorophenyl)sulfanyl]-2′-{(2R)-3-[(4-methoxyphenyl)methoxy]-2-methylpropyl}-2′,3′-dihydrospiro[cyclohexane-1,1′-indene]-4-carboxylic acid

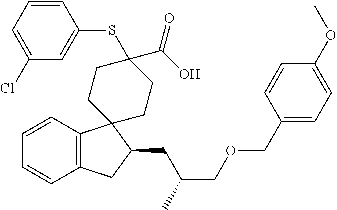 [SMILES: COc1ccc(COC[C@H](C)C[C@H]2Cc3ccccc3C23CCC(Sc2cccc(Cl)c2)(C(=O)O)CC3)cc1]

Using General procedure 10 and Example 702D as the appropriate ketone and 3-chlorobenzenethiol as the appropriate thiol, Example 718A was obtained as a mixture of diastereoisomers. ¹H NMR (500 MHz, DMSO-d₆) δ ppm: 7.56-6.83 (m, 12H), 4.38-4.31 (m, 2H), 3.73/3.72 (s/s, 3H), 3.23-3.13 (m, 2H), 2.99/2.92/2.55/2.47 (dd+dd/dd+dd, 2H), 2.48-0.91 (m, 11H), 1.78 (m, 1H), 0.91/0.83 (d/d, 3H). HRMS calculated for C₃₃H₃₇O₄SCl: 564.2101; found 582.2438 and 582.2438 (M+NH₄).

### Example 718B methyl (1r,2′S,4S)-4-[(3-chlorophenyl)sulfanyl]-2′-{(2R)-3-[(4-methoxyphenyl)methoxy]-2-methylpropyl}-2′,3′-dihydrospiro[cyclohexane-1,1′-indene]-4-carboxylate

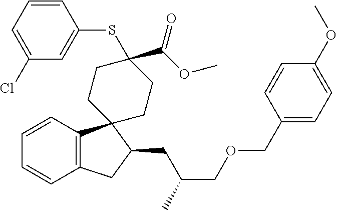 [SMILES: COc1ccc(COC[C@H](C)C[C@H]2Cc3ccccc3[C@]23CC[C@@](Sc2cccc(Cl)c2)(C(=O)OC)CC3)cc1]

Using General procedure 17a and Example 718A as the appropriate amino acid, a mixture of diastereoisomers was obtained. The diastereoisomers were separated via flash chromatography using heptane and EtOAc as eluents. The diastereoisomer eluting later was collected as Example 718B. ¹H NMR (500 MHz, DMSO-d₆) δ ppm: 7.58-7.42 (m, 4H), 7.40-7.12 (m, 4H), 7.19 (m, 2H), 6.87 (m, 2H), 4.33 (s, 2H), 3.73 (s, 3H), 3.64 (s, 3H), 3.18/3.16 (dd+dd, 2H), 2.93/2.48 (dd+dd, 2H), 2.20-0.88 (m, 11H), 2.06 (m, 1H), 0.83 (d, 3H). HRMS calculated for C₃₄H₃₉CO₄SCl: 578.2258; found 596.2580 (M+NH₄).

### Example 718C methyl (1r,2′S,4S)-4-[(3-chlorophenyl)sulfanyl]-2′-[(2R)-3-hydroxy-2-methylpropyl]-2′,3′-dihydrospiro[cyclohexane-1,1′-indene]-4-carboxylate

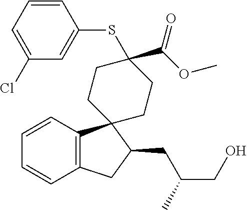 [SMILES: COC(=O)[C@]1(Sc2cccc(Cl)c2)CC[C@@]2(CC1)c1ccccc1C[C@@H]2C[C@@H](C)CO]

Using General procedure 28b and Example 718B as the appropriate PMB derivative, Example 718C was obtained. ¹H NMR (500 MHz, DMSO-d₆) δ ppm: 7.58-7.42 (m, 4H), 7.40-7.11 (m, 4H), 4.42 (t, 1H), 3.65 (s, 3H), 3.19/3.15 (m+m, 2H), 2.93/2.47 (dd+dd, 2H), 2.21-1.36 (m, 8H), 2.07 (m, 1H), 1.52 (m, 1H), 1.23/0.87 (m+m, 2H), 0.79 (d, 3H). HRMS calculated for C₂₆H₃₁O₃SCl: 458.1682; found 476.2021 (M+NH₄).

### Example 718 (1r,2′S,4S)-4-[(3-chlorophenyl)sulfanyl]-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

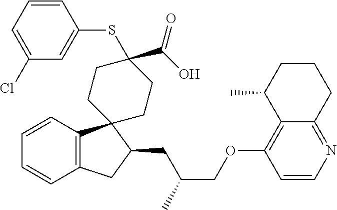 [SMILES: C[C@@H](COc1ccnc2c1[C@H](C)CCC2)C[C@H]1Cc2ccccc2[C@]12CC[C@@](Sc1cccc(Cl)c1)(C(=O)O)CC2]

Using General procedure 32 and Example 718C as the appropriate indane and Preparation 2a1 as the appropriate alcohol, Example 718 was obtained. ¹H NMR (500 MHz, DMSO-d₆) δ ppm: 12.87 (br s, 1H), 8.13 (d, 1H), 7.56-7.39 (m, 4H), 7.38-7.12 (m, 4H), 6.75 (d, 1H), 3.84/3.82 (dd+dd, 2H), 3.00 (m, 1H), 2.99/2.51 (dd+dd, 2H), 2.75/2.65 (m+m, 2H), 2.22-1.41 (m, 12H), 2.15 (m, 1H), 1.97 (m, 1H), 1.42/1.14 (m+m, 2H), 1.02 (d, 3H), 0.99 (d, 3H). HRMS calculated for C₃₅H₄₀NO₃SCl: 589.2418; found: 590.2488 (M+H).

### Example 719

### Example 719A (1s,4s)-2′-bromospiro[cyclohexane-1,1′-indene]-4-carbonitrile

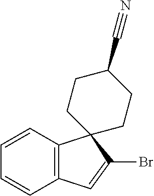 [SMILES: N#C[C@H]1CC[C@]2(CC1)C(Br)=Cc1ccccc12]

To the solution of Preparation 4a (4.25 g, 15.33 mmol) and 1-(isocyanomethylsulfonyl)-4-methyl-benzene (5.09 g, 26.06 mmol) in DME (77 mL) and EtOH (1.5 mL, 26.06 mmol) KOtBu (4.13 g, 36.8 mmol) was added portionwise at 0° C. and the reaction mixture was stirred at rt until no further conversion was observed. The reaction mixture was filtered, the solid was washed with DME. The filtrate was concentrated under reduced pressure. The crude product was purified and the diastereoisomers were separated via flash chromatography using heptane and EtOAc as eluents. The diastereoisomer eluting later was collected as Example 719A. ¹H NMR (500 MHz, DMSO-d₆) δ ppm: 7.77 (dm, 1H), 7.36 (dm, 1H), 7.28 (m, 1H), 7.18 (m, 1H), 7.07 (s, 1H), 3.38 (m, 1H), 2.25/2.03 (m+m, 4H), 2.04/1.20 (m+m, 4H). HRMS calculated for C₁₅H₁₄NBr: 287.0309; found 305.0643 (M+NH₄).

### Example 719B (1r,4R)-2′-{(2R)-3-[(4-methoxyphenyl)methoxy]-2-methylpropyl}spiro[cyclohexane-1,1′-indene]-4-carbonitrile

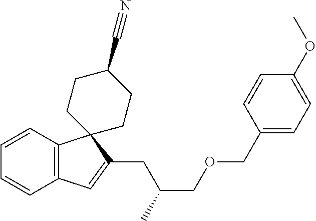 [SMILES: COc1ccc(COC[C@H](C)CC2=Cc3ccccc3[C@]23CC[C@H](C#N)CC3)cc1]

Using General procedure 27b and Example 719A as the appropriate 2-bromo-indene derivative and Preparation 3d as the appropriate Zn reagent, Example 719B was obtained. ¹H NMR (500 MHz, DMSO-d₆) δ ppm: 7.72-7.01 (m, 4H), 7.26 (m, 2H), 6.89 (m, 2H), 6.47 (t, 1H), 4.41/4.39 (d+d, 2H), 3.73 (s, 3H), 3.39 (m, 1H), 3.36/3.32 (m+m, 2H), 2.4/2.00 (m+m, 2H), 2.23/1.92 (m+m, 4H), 2.19 (m, 1H), 1.94/1.02 (m+m, 4H), 0.97 (d, 3H). HRMS calculated for C₂₇H₃₁NO₂: 401.2355; found 401.2345 (M⁺).

### Example 719C (1r,2′S,4S)-2′-{(2R)-3-[(4-methoxyphenyl)methoxy]-2-methylpropyl}-2′,3′-dihydrospiro[cyclohexane-1,1′-indene]-4-carbonitrile

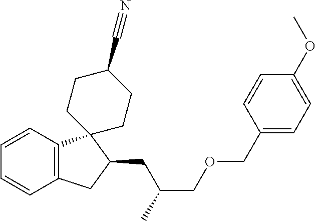 [SMILES: COc1ccc(COC[C@H](C)C[C@H]2Cc3ccccc3[C@]23CC[C@H](C#N)CC3)cc1]

Using General procedure 19 and Example 719B as the appropriate indene and using EtOAc/EtOH 1:3 instead of EtOAc, Example 719C was obtained as a mixture of diastereoisomers. The diastereoisomers were separated by prep RP-HPLC using 25 mM aq. NH₄HCO₃ solution and MeCN as eluents. The diastereoisomer eluting earlier was collected as Example 719C. ¹H NMR (500 MHz, DMSO-d₆) δ ppm: 7.24-7.08 (m, 4H), 7.19 (dm, 2H), 6.87 (dm, 2H), 4.34 (s, 2H), 3.73 (s, 3H), 3.21+3.17 (dd+dd, 2H), 2.98+2.53 (dd+dd, 2H), 2.96 (m, 1H), 2.26 (m, 1H), 2.14-0.99 (m, 11H), 0.92 (d, 3H). HRMS calculated for C₂₇H₃₃NO₂: 403.2511; found 421.2868 (M+NH₄).

### Example 719D (1s,2′S,4R)-4-[(3-chlorophenyl)methyl]-2′-{(2R)-3-[(4-methoxyphenyl)methoxy]-2-methylpropyl}-2′,3′-dihydrospiro[cyclohexane-1,1′-indene]-4-carbonitrile

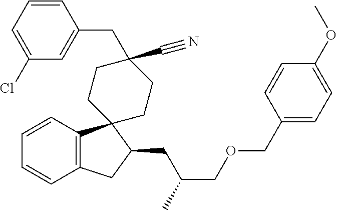 [SMILES: COc1ccc(COC[C@H](C)C[C@H]2Cc3ccccc3[C@]23CC[C@](C#N)(Cc2cccc(Cl)c2)CC3)cc1]

An oven-dried flask was charged with Example 719C (292 mg, 0.723 mmol) dissolved in dry THE (11 mL). The solution was cooled to −20° C. under N₂ atmosphere, then LDA (2 M, 0.9 mL, 1.81 mmol) was added dropwise via syringe (keeping the temperature below −20° C.), and then stirred at that temperature for 2 hours. Then 1-(bromomethyl)-3-chloro-benzene (0.11 mL, 0.868 mmol) was added dropwise. The mixture was stirred at −20° C. for 10 min, then it was allowed to warm up to rt and stirred at rt overnight. The mixture was quenched with sat. aq. NH₄Cl solution, then it was extracted with DCM. The combined organic layers were dried over Na₂SO₄, filtered and the filtrate was concentrated under reduced pressure to obtain a mixture of diastereoisomers. The diastereoisomers were separated by prep RP-HPLC using 25 mM aq. NH₄HCO₃ solution and MeCN as eluents. The diastereoisomer eluting later was collected as Example 719D. ¹H NMR (500 MHz, DMSO-d₆) δ ppm: 7.50 (d, 1H), 7.47 (t, 1H), 7.43-7.32 (m, 3H), 7.23 (dm, 2H), 7.20 (d, 1H), 7.15 (t, 1H), 7.10 (t, 1H), 6.88 (dm, 2H), 4.38 (s, 2H), 3.72 (s, 3H), 3.25 (d, 2H), 3.06 (s, 2H), 2.91+2.47 (dd+dd, 2H), 2.24-1.08 (m, 12H), 0.86 (d, 3H). HRMS calculated for C₃₄H₃₈NO₂Cl: 527.2591; found 566.2220 (M+K).

### Example 719E (1s,2′S,4R)-4-[(3-chlorophenyl)methyl]-2′-{(2R)-3-[(4-methoxyphenyl)methoxy]-2-methylpropyl}-2′,3′-dihydrospiro[cyclohexane-1,1′-indene]-4-carboxylic acid

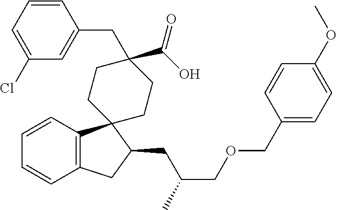 [SMILES: COc1ccc(COC[C@H](C)C[C@H]2Cc3ccccc3[C@]23CC[C@@](Cc2cccc(Cl)c2)(C(=O)O)CC3)cc1]

Example 719D (130 mg, 0.246 mmol) was dissolved in ethylene glycol (1.2 mL). KOH (83 mg, 1.477 mmol) was added. The reaction mixture was then warmed up to 180° C. and stirred at that temperature. After 1 day stirring KOH (83 mg, 1.477 mmol) was added again. The reaction mixture was stirred until no further conversion was observed. During the reaction the PMB protecting group was partly (˜50%) removed. Then it was cooled to rt, and diluted with water. The pH was set to 2-3 with 2 M aq. HCl solution, and then it was extracted with DCM. The combined organic layers were dried over Na₂SO₄, filtered and the filtrate was concentrated under reduced pressure to obtain Example 719E, which was used in the next step without further purification. HRMS calculated for C₃₄H₃₉O₄Cl: 546.2537; found 564.2864 (M+NH₄).

### Example 719F methyl (1s,2′S,4R)-4-[(3-chlorophenyl)methyl]-2′-{(2R)-3-[(4-methoxyphenyl)methoxy]-2-methylpropyl}-2′,3′-dihydrospiro[cyclohexane-1,1′-indene]-4-carboxylate

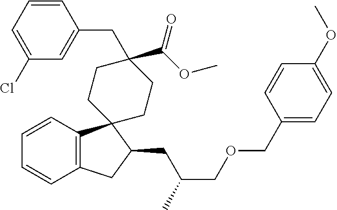 [SMILES: COc1ccc(COC[C@H](C)C[C@H]2Cc3ccccc3[C@]23CC[C@@](Cc2cccc(Cl)c2)(C(=O)OC)CC3)cc1]

Using General procedure 17a and Example 719E as the appropriate amino acid, Example 719F was obtained. HRMS calculated for C₃₅H₄₁O₄Cl: 560.2693; found 578.3041 (M+NH₄).

### Example 719G methyl (1s,2′S,4R)-4-[(3-chlorophenyl)methyl]-2′-[(2R)-3-hydroxy-2-methylpropyl]-2′,3′-dihydrospiro[cyclohexane-1,1′-indene]-4-carboxylate

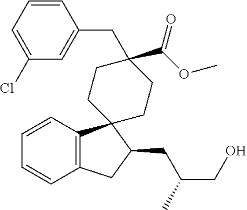 [SMILES: COC(=O)[C@]1(Cc2cccc(Cl)c2)CC[C@@]2(CC1)c1ccccc1C[C@@H]2C[C@@H](C)CO]

Using General procedure 28b and Example 719F as the appropriate PMB derivative, Example 719G was obtained. ¹H NMR (500 MHz, DMSO-d₆) δ ppm: 7.46-7.04 (m, 4H), 7.36-7.04 (m, 4H), 4.44 (t, 1H), 3.62 (s, 3H), 3.20 (m, 2H), 2.95/2.91 (d+d, 2H), 2.89/2.45 (dd+dd, 2H), 2.00-1.30 (m, 8H), 1.93 (m, 1H), 1.51 (m, 1H), 1.33/0.88 (m+m, 2H), 0.79 (d, 3H). HRMS calculated for C₂₇H₃₃O₃Cl: 440.2118; found 458.2444 (M+NH₄).

### Example 719 (1s,2′S,4R)-4-[(3-chlorophenyl)methyl]-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylic acid

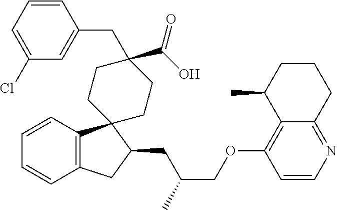 [SMILES: C[C@@H](COc1ccnc2c1[C@@H](C)CCC2)C[C@H]1Cc2ccccc2[C@]12CC[C@@](Cc1cccc(Cl)c1)(C(=O)O)CC2]

Using General procedure 32 and Example 719G as the appropriate indane and Preparation 2a2 as the appropriate alcohol, Example 719 was obtained. ¹H NMR (500 MHz, DMSO-d₆) δ ppm: 12.49 (br s, 1H), 8.14 (d, 1H), 7.38 (d, 1H), 7.31 (t, 1H), 7.30 (dd, 1H), 7.26 (t, 1H), 7.18 (d, 1H), 7.16 (dd, 1H), 7.12 (t, 1H), 7.06 (t, 1H), 6.76 (d, 1H), 3.89/3.82 (dd+dd, 2H), 3.03 (m, 1H), 2.95/2.50 (dd+dd, 2H), 2.93/2.88 (d+d, 2H), 2.76/2.65 (m+m, 2H), 2.03 (dd, 1H), 1.99-1.18 (m, 8H), 1.97 (m, 1H), 1.80/1.74 (m+m, 2H), 1.64 (m, 2H), 1.42/1.22 (dd+dd, 2H), 1.15 (d, 3H), 0.99 (d, 3H). HRMS calculated for C₃₆H₄₂NO₃Cl: 571.2853; found: 572.2918 (M+H).

### Example 721 and Example 722

### Example 721A methyl (1r,4R)-6′-acetyl-4-(3-chloroanilino)-2′-{(2R)-2-methyl-3-[(thieno[3,2-b]pyridin-7-yl)oxy]propyl}-2′,3′-dihydrospiro[cyclohexane-1,1′-indene]-4-carboxylate

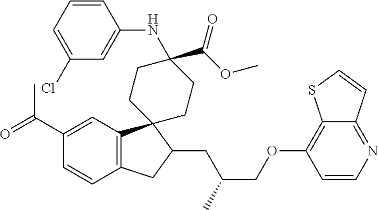

Using General procedure 30a and a mixture of Preparation 17a and Preparation 17b as the appropriate indane and thieno[3,2-b]pyridin-7-ol as the appropriate alcohol, Example 721A was obtained as a mixture of diastereoisomers. ¹H NMR (500 MHz, DMSO-d₆) δ ppm: 8.89 (d, 1H), 8.43/8.30 (d/d, 1H), 7.93/7.92 (s/s, 1H), 7.79 (d, 1H), 7.73/7.68 (d/d, 1H), 7.49/7.47 (d/d, 1H), 7.33 (d, 1H), 7.04/7.01 (t/t, 1H), 6.61/6.53 (m/m, 1H), 6.54/6.52 (d/d, 1H), 6.45/6.39 (d/d, 1H), 4.43/4.36 (m/m, 2H), 3.66/3.65 (s/s, 3H), 3.10/2.64 (m+m, 2H), 2.55 (s, 3H), 2.52-1.22 (m, 10H), 2.24 (m/m, 1H), 2.20/2.12 (m/m, 1H), 1.11/1.09 (d/d, 3H) HRMS calculated for C₃₅H₃₇N₂O₄SCl: 616.2162; found: 617.2241 (M+H).

### Example 721 (1r,2′R,4R)-4-(3-chloroanilino)-6′-(1-hydroxyethyl)-2′-{(2R)-2-methyl-3-[(thieno[3,2-b]pyridin-7-yl)oxy]propyl}-2′,3′-dihydrospiro[cyclohexane-1,1′-indene]-4-carboxylic acid

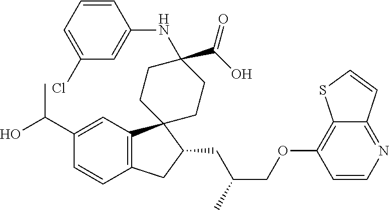 [SMILES: CC(O)c1ccc2c(c1)[C@]1(CC[C@@](Nc3cccc(Cl)c3)(C(=O)O)CC1)[C@H](C[C@@H](C)COc1ccnc3ccsc13)C2]

and

### Example 722 (1r,2′S,4S)-4-(3-chloroanilino)-6′-(1-hydroxyethyl)-2′-{(2R)-2-methyl-3-[(thieno[3,2-b]pyridin-7-yl)oxy]propyl}-2′,3′-dihydrospiro[cyclohexane-1,1′-indene]-4-carboxylic acid

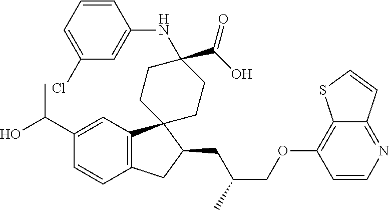 [SMILES: CC(O)c1ccc2c(c1)[C@]1(CC[C@@](Nc3cccc(Cl)c3)(C(=O)O)CC1)[C@@H](C[C@@H](C)COc1ccnc3ccsc13)C2]

The diastereoisomers of Example 721A were separated by chiral chromatography. Column: OD, 50×500 mm, 20 μm, Eluents: EtOH/heptane. The diastereoisomer eluting earlier was treated as described in General procedure 36, then hydrolyzed according to General procedure 33a to obtain Example 721 as a mixture of diastereoisomers. ¹H NMR (500 MHz, DMSO-d₆) δ ppm: 12.59 (br s, 1H), 8.50 (d, 1H), 7.91 (d, 1H), 7.48 (d, 1H), 7.37/7.35 (br s, 1H), 7.14-7.07 (m, 2H), 7.03 (t, 1H), 7.00 (d, 1H), 6.60 (t, 1H), 6.54 (dm, 1H), 6.52 (dm, 1H), 6.09 (br s, 1H), 5.08 (d, 1H), 4.67 (m, 1H), 4.25/4.13 (dd+dd, 2H), 3.02/2.57 (dd+dd, 2H), 2.49-1.34 (m, 8H), 2.21 (m, 1H), 2.14 (m, 1H), 1.69/1.25 (m+m, 2H), 1.30 (d, 3H), 1.10 (d, 3H). HRMS calculated for C₃₄H₃₇N₂O₄SCl: 604.2162; found: 605.2236 (M+H).

The diastereoisomer eluting later was treated as described in General procedure 36, then hydrolyzed according to General procedure 33a to obtain Example 722 as a mixture of diastereoisomers. ¹H NMR (500 MHz, DMSO-d₆) δ ppm: 12.64 (br s, 1H), 8.51 (d, 1H), 8.01 (d, 1H), 7.50 (d, 1H), 7.36/7.34 (br s, 1H), 7.14-7.07 (m, 2H), 7.05 (t, 1H), 6.99 (d, 1H), 6.64 (t, 1H), 6.56 (dm, 1H), 6.54 (dm, 1H), 6.24 (br s, 1H), 5.08 (d, 1H), 4.67 (m, 1H), 4.16/4.10 (dd+dd, 2H), 2.98/2.52 (dd+dd, 2H), 2.52-1.37 (m, 8H), 2.18 (m, 1H), 2.07 (m, 1H), 1.43/1.37 (m+m, 2H), 1.30 (d, 3H), 1.07 (d, 3H). HRMS calculated for C₃₄H₃₇N₂O₄SCl: 604.2162; found: 605.2232 (M+H).

### Example 729 (1r,2′R,4R)-4-(3-chloroanilino)-6′-(2-methoxyethoxy)-2′-{(2R)-2-methyl-3-[(thieno[3,2-b]pyridin-7-yl)oxy]propyl}-2′,3′-dihydrospiro[cyclohexane-1,1′-indene]-4-carboxylic acid

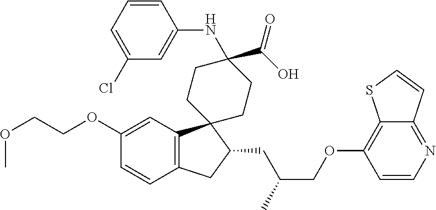 [SMILES: COCCOc1ccc2c(c1)[C@]1(CC[C@@](Nc3cccc(Cl)c3)(C(=O)O)CC1)[C@H](C[C@@H](C)COc1ccnc3ccsc13)C2]

Using General procedure 32 and Preparation 19b as the appropriate indane and 2-methoxyethanol as the appropriate alcohol Example 729 was obtained. HRMS calculated for C₃₅H₃₉N₂O₅SCl: 634.2268; found: 635.2330 (M+H).

### Example 730 (1r,2′S,4S)-4-(3-chloroanilino)-6′-(2-methoxyethoxy)-2′-{(2R)-2-methyl-3-[(thieno[3,2-b]pyridin-7-yl)oxy]propyl}-2′,3′-dihydrospiro[cyclohexane-1,1′-indene]-4-carboxylic acid

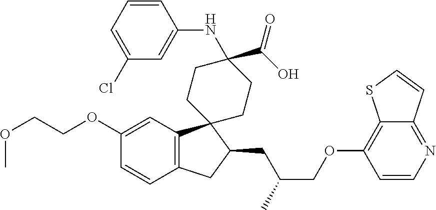 [SMILES: COCCOc1ccc2c(c1)[C@]1(CC[C@@](Nc3cccc(Cl)c3)(C(=O)O)CC1)[C@@H](C[C@@H](C)COc1ccnc3ccsc13)C2]

Using General procedure 32 and Preparation 19a as the appropriate indane and 2-methoxyethanol as the appropriate alcohol Example 730 was obtained. ¹H NMR (500 MHz, DMSO-d₆) δ ppm: 12.72 (br s, 1H), 8.51 (d, 1H), 8.01 (d, 1H), 7.50 (d, 1H), 7.09 (d, 1H), 7.04 (t, 1H), 6.99 (d, 1H), 6.91 (d, 1H), 6.73 (dd, 1H), 6.61 (t, 1H), 6.53 (m, 2H), 6.21 (br s, 1H), 4.16/4.10 (dd+dd, 2H), 4.04 (m, 2H), 3.65 (t, 2H), 3.31 (s, 3H), 2.93/2.46 (dd+dd, 2H), 2.42-1.35 (m, 8H), 2.13 (m, 1H), 2.05 (m, 1H), 1.47/1.35 (m+m, 2H), 1.06 (d, 3H). HRMS calculated for C₃₅H₃₉N₂O₅SCl: 634.2268; found: 635.2333 (M+H).

### Example 733 and Example 734

### Example 733A methyl (1s,4s)-2′-bromo-4-(3-chloroanilino)-5′,6′-dimethylspiro[cyclohexane-1,1′-indene]-4-carboxylate

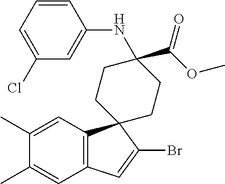

Using General procedure 17a and Example 140 as the appropriate amino acid Example 733A was obtained. ¹H NMR (500 MHz, DMSO-d₆) δ ppm: 7.47 (br s, 1H), 7.13 (br s, 1H), 7.09 (t, 1H), 6.94 (s, 1H), 6.61 (m, 1H), 6.59 (dm, 1H), 6.48 (br s, 1H), 6.47 (dm, 1H), 3.69 (s, 3H), 2.40 (td+m, 4H), 2.26 (s, 3H), 2.22 (s, 3H), 2.17/0.95 (td+m, 4H). HRMS calculated for C₂₄H₂₅NO₂BrCl: 473.0757; found: 474.0820 (M+H).

### Example 733B methyl (1r,4R)-4-(3-chloroanilino)-2′-{(2R)-3-[(4-methoxyphenyl)methoxy]-2-methylpropyl}-5′,6′-dimethylspiro[cyclohexane-1,1′-indene]-4-carboxylate

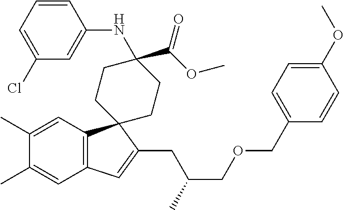 [SMILES: COc1ccc(COC[C@H](C)CC2=Cc3cc(C)c(C)cc3[C@]23CC[C@@](Nc2cccc(Cl)c2)(C(=O)OC)CC3)cc1]

Using General procedure 27a and Example 733A as the appropriate 2-bromo-indene derivative and Preparation 3d as the appropriate Zn reagent Example 733B was obtained. ¹H NMR (500 MHz, DMSO-d₆) δ ppm: 7.41 (s, 1H), 7.21 (dm, 2H), 7.09 (t, 1H), 7.03 (s, 1H), 6.86 (dm, 2H), 6.63 (t, 1H), 6.59 (dd, 1H), 6.46 (dd, 1H), 6.40 (s, 1H), 6.33 (s, 1H), 4.38/4.36 (d+d, 2H), 3.72 (s, 3H), 3.69 (s, 3H), 3.32/3.26 (dd+dd, 2H), 2.39/2.17/2.06/0.82 (m, 8H), 2.30/1.95 (dd+dd, 2H), 2.24 (s, 3H), 2.20 (s, 3H), 2.13 (m, 1H), 0.93 (d, 3H). HRMS calculated for C₃₆H₄₂NO₄Cl: 587.2802; found: 588.2876 (M+H).

### Example 733C methyl (1r,4R)-4-(3-chloroanilino)-2′-{(2R)-3-[(4-methoxyphenyl)methoxy]-2-methylpropyl}-5′,6′-dimethyl-2′,3′-dihydrospiro[cyclohexane-1,1′-indene]-4-carboxylate

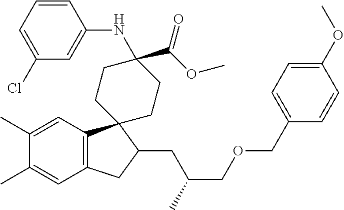

Using General procedure 19 and Example 733B as the appropriate indene Example 733C was obtained as a mixture of diastereoisomers. ¹H NMR (500 MHz, DMSO-d₆) δ ppm: 7.20 (m, 2H), 7.10/7.09 (s, 1H), 7.06/7.05 (t, 1H), 6.94/6.93 (s, 1H), 6.86/6.79 (m, 2H), 6.61/6.59 (t, 1H), 6.57/6.56 (dm, 1H), 6.47/6.45 (dm, 1H), 6.31/6.29 (s, 1H), 4.38/4.36/4.35/4.34 (d+d, 2H), 3.72/3.68 (s, 3H), 3.65 (s, 3H), 3.35-3.11 (dd+dd, 2H), 2.84/2.42 (dd+dd, 2H), 2.50-0.95 (m, 11H), 2.19 (s, 3H), 2.15 (s, 3H), 2.03 (m, 1H), 0.93/0.88 (d, 3H). HRMS calculated for C₃₆H₄₄NO₄Cl: 589.2959; found: 612.2833 and 612.2850 (M+Na).

### Example 733D methyl (1r,2′S,4S)-4-(3-chloroanilino)-2′-[(2R)-3-hydroxy-2-methylpropyl]-5′,6′-dimethyl-2′,3′-dihydrospiro[cyclohexane-1,1′-indene]-4-carboxylate

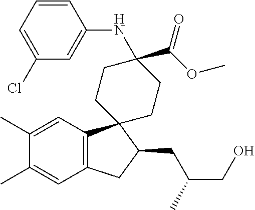 [SMILES: COC(=O)[C@]1(Nc2cccc(Cl)c2)CC[C@@]2(CC1)c1cc(C)c(C)cc1C[C@@H]2C[C@@H](C)CO]

and

### Example 733E methyl (1r,2′R,4R)-4-(3-chloroanilino)-2′-[(2R)-3-hydroxy-2-methylpropyl]-5′,6′-dimethyl-2′,3′-dihydrospiro[cyclohexane-1,1′-indene]-4-carboxylate

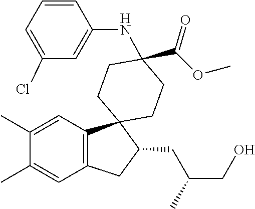 [SMILES: COC(=O)[C@]1(Nc2cccc(Cl)c2)CC[C@@]2(CC1)c1cc(C)c(C)cc1C[C@H]2C[C@@H](C)CO]

Using General procedure 28b and Example 733C as the appropriate PMB derivative a mixture of diastereoisomers was obtained. The diastereoisomers were separated via prep RP-HPLC using 25 mM aq. NH₄HCO₃ solution and MeCN as eluents. The diastereoisomer eluting earlier was collected as Example 733D. ¹H NMR (500 MHz, DMSO-d₆) δ ppm: 7.10 (s, 1H), 7.06 (t, 1H), 6.95 (s, 1H), 6.59 (t, 1H), 6.56 (dm, 1H), 6.44 (dm, 1H), 6.29 (s, 1H), 4.43 (t, 1H), 3.65 (s, 3H), 3.20 (m, 2H), 2.85/2.40 (dd+dd, 2H), 2.50-0.96 (m, 8H), 2.19 (s, 3H), 2.15 (s, 3H), 2.05 (m, 1H), 1.55 (m, 1H), 1.30/1.03 (m+m, 2H), 0.84 (d, 3H). HRMS calculated for C₂₈H₃₆NO₃Cl: 469.2384; found: 470.2460 (M+H).

The diastereoisomer eluting later was collected as Example 733E. ¹H NMR (500 MHz, DMSO-d₆) δ ppm: 7.10 (s, 1H), 7.06 (t, 1H), 6.95 (s, 1H), 6.59 (t, 1H), 6.56 (dm, 1H), 6.45 (dm, 1H), 6.27 (s, 1H), 4.38 (t, 1H), 3.64 (s, 3H), 3.41/3.18 (m+m, 2H), 2.88/2.45 (dd+dd, 2H), 2.50-1.23 (m, 8H), 2.19 (s, 3H), 2.15 (s, 3H), 2.05 (m, 1H), 1.58 (m, 1H), 1.41/0.97 (m+m, 2H), 0.89 (d, 3H). HRMS calculated for C₂₈H₃₆NO₃Cl: 469.2384; found: 470.2445 (M+H).

### Example 733 (1r,2′S,4S)-4-(3-chloroanilino)-5′,6′-dimethyl-2′-{(2R)-2-methyl-3-[(thieno[3,2-b]pyridin-7-yl)oxy]propyl}-2′,3′-dihydrospiro[cyclohexane-1,1′-indene]-4-carboxylic acid

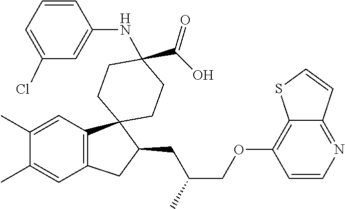 [SMILES: Cc1cc2c(cc1C)[C@]1(CC[C@@](Nc3cccc(Cl)c3)(C(=O)O)CC1)[C@@H](C[C@@H](C)COc1ccnc3ccsc13)C2]

Using General procedure 32 and Example 733D as the appropriate indane and thieno[3,2-b]pyridin-7-ol as the appropriate alcohol Example 733 was obtained. ¹H NMR (500 MHz, DMSO-d₆) δ ppm: 12.61 (br s, 1H), 8.51 (d, 1H), 8.01 (d, 1H), 7.50 (d, 1H), 7.10 (s, 1H), 7.01 (t, 1H), 6.99 (d, 1H), 6.95 (s, 1H), 6.62 (t, 1H), 6.54 (dm, 2H), 6.21 (br s, 1H), 4.15/4.09 (dd+dd, 2H), 2.92/2.47 (dd+dd, 2H), 2.55-1.20 (m, 8H), 2.18 (s, 3H), 2.15 (s, 3H), 2.13 (m, 1H), 2.06 (m, 1H), 1.43/1.36 (m+m, 2H), 1.06 (d, 3H). HRMS calculated for C₃₄H₃₇N₂O₃SCl: 588.2213; found: 589.2278 (M+H).

### Example 734 (1r,2′R,4R)-4-(3-chloroanilino)-5′,6′-dimethyl-2′-{(2R)-2-methyl-3-[(thieno[3,2-b]pyridin-7-yl)oxy]propyl}-2′,3′-dihydrospiro[cyclohexane-1,1′-indene]-4-carboxylic acid

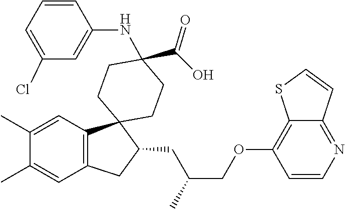 [SMILES: Cc1cc2c(cc1C)[C@]1(CC[C@@](Nc3cccc(Cl)c3)(C(=O)O)CC1)[C@H](C[C@@H](C)COc1ccnc3ccsc13)C2]

Using General procedure 32 and Example 733E as the appropriate indane and thieno[3,2-b]pyridin-7-ol as the appropriate alcohol Example 734 was obtained. ¹H NMR (500 MHz, DMSO-d₆) δ ppm: 12.65 (br s, 1H), 8.50 (d, 1H), 7.91 (d, 1H), 7.48 (d, 1H), 7.12 (s, 1H), 7.03 (t, 1H), 6.99 (d, 1H), 6.96 (s, 1H), 6.59 (t, 1H), 6.54 (dm, 1H), 6.50 (dm, 1H), 6.08 (br s, 1H), 4.23/4.12 (dd+dd, 2H), 2.96/2.52 (dd+dd, 2H), 2.45-1.29 (m, 8H), 2.18 (s, 3H), 2.15 (s, 3H), 2.15 (m, 1H), 2.13 (m, 1H), 1.67/1.24 (m+m, 2H), 1.09 (d, 3H). HRMS calculated for C₃₄H₃₇N₂O₃SCl: 588.2213; found: 589.2278 (M+H).

### Example 735 and Example 736

### Example 735A methyl (1s,4s)-2′-bromo-4-(3-chloroanilino)-6′-fluorospiro[cyclohexane-1,1′-indene]-4-carboxylate

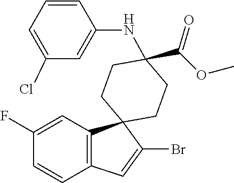 [SMILES: COC(=O)[C@]1(Nc2cccc(Cl)c2)CC[C@]2(CC1)C(Br)=Cc1ccc(F)cc12]

Using General procedure 17a and Example 139 as the appropriate amino acid Example 735A was obtained. ¹H NMR (500 MHz, DMSO-d₆) δ ppm: 7.49 (dd, 1H), 7.39 (dd, 1H), 7.17 (m, 1H), 7.10 (t, 1H), 7.03 (s, 1H), 6.62 (t, 1H), 6.60 (dm, 1H), 6.49 (s, 1H), 6.48 (dm, 1H), 3.69 (s, 3H), 2.35/2.27 (m+m, 4H), 2.20/1.01 (m+m, 4H). HRMS calculated for C₂₂H₂₀NO₂FClBr: 463.0350; found: 464.0413 (M+H).

### Example 735B methyl (1r,4R)-4-(3-chloroanilino)-6′-fluoro-2′-{(2R)-3-[(4-methoxyphenyl)methoxy]-2-methylpropyl}spiro[cyclohexane-1,1′-indene]-4-carboxylate

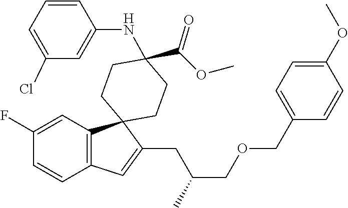 [SMILES: COc1ccc(COC[C@H](C)CC2=Cc3ccc(F)cc3[C@]23CC[C@@](Nc2cccc(Cl)c2)(C(=O)OC)CC3)cc1]

Using General procedure 27a and Example 735A as the appropriate 2-bromo-indene derivative and Preparation 3d as the appropriate Zn reagent Example 735B was obtained. ¹H NMR (500 MHz, DMSO-d₆) δ ppm: 7.39 (dd, 1H), 7.27 (dd, 1H), 7.21 (dm, 2H), 7.09 (t, 1H), 7.07 (td, 1H), 6.85 (dm, 2H), 6.64 (t, 1H), 6.60 (dd, 1H), 6.47 (dd, 1H), 6.43 (s, 1H), 6.42 (s, 1H), 4.40/4.36 (d+d, 2H), 3.72 (s, 3H), 3.69 (s, 3H), 3.32/3.28 (dd+dd, 2H), 2.34/1.96 (dd+dd, 2H), 2.34/2.21/2.10/0.88 (m+m+m+m, 8H), 2.15 (m, 1H), 0.93 (d, 3H). HRMS calculated for C₃₄H₃₇NO₄FCl: 577.2395; found: 578.2463 (M+H).

### Example 735C methyl (1r,4R)-4-(3-chloroanilino)-6′-fluoro-2′-{(2R)-3-[(4-methoxyphenyl)methoxy]-2-methylpropyl}-2′,3′-dihydrospiro[cyclohexane-1,1′-indene]-4-carboxylate

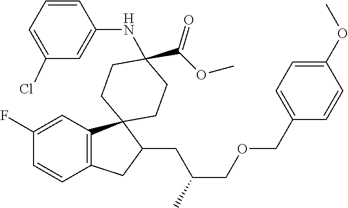

Using General procedure 19 and Example 735B as the appropriate indene Example 735C was obtained as a mixture of diastereoisomers. ¹H NMR (500 MHz, DMSO-d₆) δ ppm: 7.18 (m, 3H), 7.09 (m, 1H), 7.06/7.05 (t/t, 1H), 6.94 (m, 1H), 6.84/6.77 (dm/dm, 2H), 6.59 (t, 1H), 6.56 (dm, 1H), 6.45/6.44 (dm/dm, 1H), 6.30/6.28 (s/s, 1H), 4.40-4.28 (m, 2H), 3.70/3.66 (s/s, 3H), 3.63 (s, 3H), 3.30/3.24/3.20 (dd+dd/d, 2H), 2.89/2.44 (dd+m, 2H), 2.44-1.25 (m, 8H), 2.08 (m, 1H), 1.74 (m, 1H), 1.46/1.30/1.12/1.00 (m+m/m+m, 2H), 0.91/0.86 (d/d, 3H). HRMS calculated for C₃₄H₃₉NO₄FCl: 579.2552; found: 580.2586 and 580.2597 (M+H).

### Example 735D methyl (1r,2′S,4S)-4-(3-chloroanilino)-6′-fluoro-2′-[(2R)-3-hydroxy-2-methylpropyl]-2′,3′-dihydrospiro[cyclohexane-1,1′-indene]-4-carboxylate

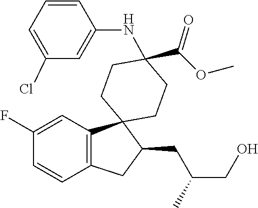 [SMILES: COC(=O)[C@]1(Nc2cccc(Cl)c2)CC[C@@]2(CC1)c1cc(F)ccc1C[C@@H]2C[C@@H](C)CO]

and

### Example 735E methyl (1r,2′R,4R)-4-(3-chloroanilino)-6′-fluoro-2′-[(2R)-3-hydroxy-2-methylpropyl]-2′,3′-dihydrospiro[cyclohexane-1,1′-indene]-4-carboxylate

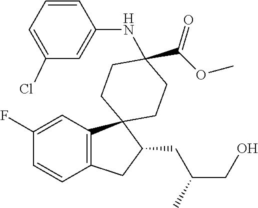 [SMILES: COC(=O)[C@]1(Nc2cccc(Cl)c2)CC[C@@]2(CC1)c1cc(F)ccc1C[C@H]2C[C@@H](C)CO]

Using General procedure 28b and Example 735C as the appropriate PMB derivative a mixture of diastereoisomers was obtained. The diastereoisomers were separated via prep RP-HPLC using 25 mM aq. NH₄HCO₃ solution and MeCN as eluents. The diastereoisomer eluting earlier was collected as Example 735D. HRMS calculated for C₂₆H₃₁NO₃FCl: 459.1977; found: 460.2043 (M+H).

The diastereoisomer eluting later was collected as Example 735E. HRMS calculated for C₂₆H₃₁NO₃FCl: 459.1977; found: 460.2052 (M+H).

### Example 735 (1r,2′S,4S)-4-(3-chloroanilino)-6′-fluoro-2′-{(2R)-2-methyl-3-[(thieno[3,2-b]pyridin-7-yl)oxy]propyl}-2′,3′-dihydrospiro[cyclohexane-1,1′-indene]-4-carboxylic acid

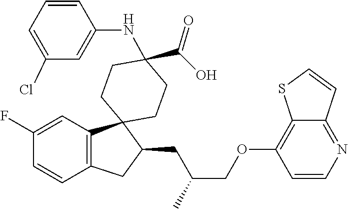 [SMILES: C[C@@H](COc1ccnc2ccsc12)C[C@H]1Cc2ccc(F)cc2[C@]12CC[C@@](Nc1cccc(Cl)c1)(C(=O)O)CC2]

Using General procedure 32 and Example 735D as the appropriate indane and thieno[3,2-b]pyridin-7-ol as the appropriate alcohol Example 735 was obtained. ¹H NMR (500 MHz, DMSO-d₆) δ ppm: 12.70 (br s, 1H), 8.51 (d, 1H), 8.01 (d, 1H), 7.50 (d, 1H), 7.21 (dd, 1H), 7.10 (dd, 1H), 7.04 (t, 1H), 7.00 (d, 1H), 6.96 (td, 1H), 6.62 (t, 1H), 6.54 (m, 2H), 6.23 (br s, 1H), 4.16/4.11 (dd+dd, 2H), 2.99/2.51 (dd+dd, 2H), 2.40/2.08/1.99/1.89/1.86/1.74/1.50/1.38 (m, 8H), 2.20 (m, 1H), 2.06 (m, 1H), 1.46/1.36 (m+m, 2H), 1.06 (d, 3H). HRMS calculated for C₃₂H₃₂N₂O₃FSCl: 578.1806; found: 579.1868 (M+H).

### Example 736 (1r,2′R,4R)-4-(3-chloroanilino)-6′-fluoro-2′-{(2R)-2-methyl-3-[(thieno[3,2-b]pyridin-7-yl)oxy]propyl}-2′,3′-dihydrospiro[cyclohexane-1,1′-indene]-4-carboxylic acid

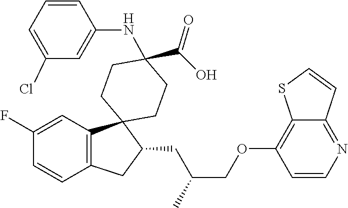 [SMILES: C[C@@H](COc1ccnc2ccsc12)C[C@@H]1Cc2ccc(F)cc2[C@]12CC[C@@](Nc1cccc(Cl)c1)(C(=O)O)CC2]

Using General procedure 32 and Example 735E as the appropriate indane and thieno[3,2-b]pyridin-7-ol as the appropriate alcohol Example 736 was obtained. ¹H NMR (500 MHz, DMSO-d₆) δ ppm: 12.71 (br s, 1H), 8.50 (d, 1H), 7.91 (d, 1H), 7.48 (d, 1H), 7.22 (dd, 1H), 7.12 (dd, 1H), 7.04 (t, 1H), 7.00 (d, 1H), 6.96 (m, 1H), 6.59 (t, 1H), 6.54 (dm, 1H), 6.50 (dm, 1H), 6.12 (br s, 1H), 4.24/4.13 (dd+dd, 2H), 3.02/2.57 (dd+dd, 2H), 2.43-1.25 (m, 8H), 2.23 (m, 1H), 2.13 (m, 1H), 1.69/1.28 (m+m, 2H), 1.10 (d, 3H). HRMS calculated for C₃₂H₃₂N₂O₃FSCl: 578.1806; found: 579.1877 (M+H).

### Example 737 and Example 738

### Example 737A methyl (1s,4s)-2′-bromo-4-(3-chloroanilino)-5′-fluorospiro[cyclohexane-1,1′-indene]-4-carboxylate

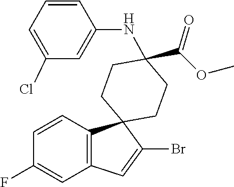 [SMILES: COC(=O)[C@]1(Nc2cccc(Cl)c2)CC[C@]2(CC1)C(Br)=Cc1cc(F)ccc12]

Using General procedure 17a and Example 138 as the appropriate amino acid Example 737A was obtained. ¹H NMR (500 MHz, DMSO-d₆) δ ppm: 7.70 (dd, 1H), 7.22 (dd, 1H), 7.10 (t, 1H), 7.06 (m, 1H), 7.04 (s, 1H), 6.61 (t, 1H), 6.60 (dm, 1H), 6.50 (s, 1H), 6.47 (dm, 1H), 3.68 (s, 3H), 2.36/2.27 (m+m, 4H), 2.19/0.98 (m+m, 4H). HRMS calculated for C₂₂H₂₀BrClFNO₂: 463.035; found 464.0426 (M+H).

### Example 737B methyl (1r,4R)-4-(3-chloroanilino)-5′-fluoro-2′-{(2R)-3-[(4-methoxyphenyl)methoxy]-2-methylpropyl}spiro[cyclohexane-1,1′-indene]-4-carboxylate

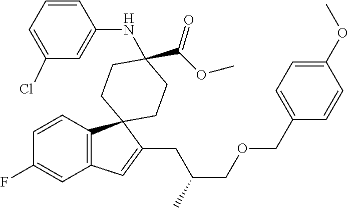 [SMILES: COc1ccc(COC[C@H](C)CC2=Cc3cc(F)ccc3[C@]23CC[C@@](Nc2cccc(Cl)c2)(C(=O)OC)CC3)cc1]

Using General procedure 27a and Example 737A as the appropriate 2-bromo-indene derivative and Preparation 3d as the appropriate Zn reagent Example 737B was obtained. ¹H NMR (500 MHz, DMSO-d₆) δ ppm: 7.61 (dd, 1H), 7.21 (dm, 2H), 7.09 (m, 2H), 6.91 (m, 1H), 6.86 (dm, 2H), 6.63 (t, 1H), 6.60 (dm, 1H), 6.47 (dm, 1H), 6.44 (br s, 1H), 6.42 (s, 1H), 4.39/4.36 (d+d, 2H), 3.72 (s, 3H), 3.68 (s, 3H), 3.30 (m, 2H), 2.41-0.81 (m, 8H), 2.36/1.99 (dm+dd, 2H), 2.15 (m, 1H), 0.93 (d, 3H). HRMS calculated for C₃₄H₃₇NO₄FCl: 577.2395; found: 578.2451 (M+H).

### Example 737C methyl (1r,4R)-4-(3-chloroanilino)-5′-fluoro-2′-{(2R)-3-[(4-methoxyphenyl)methoxy]-2-methylpropyl}-2′,3′-dihydrospiro[cyclohexane-1,1′-indene]-4-carboxylate

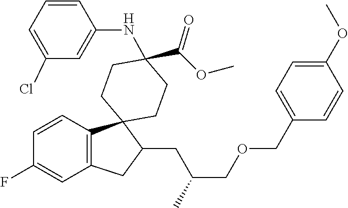

Using General procedure 19 and Example 737B as the appropriate indene Example 737C was obtained as a mixture of diastereoisomers. ¹H NMR (500 MHz, DMSO-d₆) δ ppm: 7.36 (dd, 1H), 7.20 (m, 2H), 7.06 (t, 1H), 7.01 (dd, 1H), 6.96 (m, 1H), 6.86/6.80 (m, 2H), 6.64-6.40 (m, 3H), 6.33/6.31 (s, 1H), 4.43-4.30 (d+d, 2H), 3.76-3.62 (s, 6H), 3.38-3.10 (m, 2H), 2.93/2.50 (dd+dd, 2H), 2.50-0.76 (m, 11H), 2.09 (m, 1H), 0.93/0.88 (d, 3H). HRMS calculated for C₃₄H₃₉NO₄FCl: 579.2552; found: 618.2132 and 618.2132 (M+K).

### Example 737D methyl (1r,2′S,4S)-4-(3-chloroanilino)-5′-fluoro-2′-[(2R)-3-hydroxy-2-methylpropyl]-2′,3′-dihydrospiro[cyclohexane-1,1′-indene]-4-carboxylate

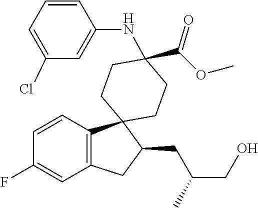 [SMILES: COC(=O)[C@]1(Nc2cccc(Cl)c2)CC[C@@]2(CC1)c1ccc(F)cc1C[C@@H]2C[C@@H](C)CO]

and

### Example 737E methyl (1r,2′R,4R)-4-(3-chloroanilino)-5′-fluoro-2′-[(2R)-3-hydroxy-2-methylpropyl]-2′,3′-dihydrospiro[cyclohexane-1,1′-indene]-4-carboxylate

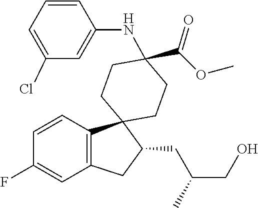 [SMILES: COC(=O)[C@]1(Nc2cccc(Cl)c2)CC[C@@]2(CC1)c1ccc(F)cc1C[C@H]2C[C@@H](C)CO]

Using General procedure 28b and Example 737C as the appropriate PMB derivative a mixture of diastereoisomers was obtained. The diastereoisomers were separated via prep RP-HPLC using 25 mM aq. NH₄HCO₃ solution and MeCN as eluents. The diastereoisomer eluting earlier was collected as Example 737D. LRMS calculated for C₂₆H₃₁NO₃FCl: 459.2; found: 460.4 (M+H).

The diastereoisomer eluting later was collected as Example 737E. LRMS calculated for C₂₆H₃₁NO₃FCl: 459.2; found: 460.4 (M+H).

### Example 737 (1r,2′S,4S)-4-(3-chloroanilino)-5′-fluoro-2′-{(2R)-2-methyl-3-[(thieno[3,2-b]pyridin-7-yl)oxy]propyl}-2′,3′-dihydrospiro[cyclohexane-1,1′-indene]-4-carboxylic acid

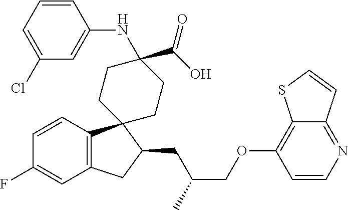 [SMILES: C[C@@H](COc1ccnc2ccsc12)C[C@H]1Cc2cc(F)ccc2[C@]12CC[C@@](Nc1cccc(Cl)c1)(C(=O)O)CC2]

Using General procedure 32 and Example 737D as the appropriate indane and thieno[3,2-b]pyridin-7-ol as the appropriate alcohol Example 737 was obtained. ¹H NMR (500 MHz, DMSO-d₆) δ ppm: 8.50 (d, 1H), 8.01 (d, 1H), 7.57 (dd, 1H), 7.50 (d, 1H), 7.03 (t, 1H), 7.02 (m, 1H), 7.00 (d, 1H), 6.97 (m, 1H), 6.62 (t, 1H), 6.54 (dd, 1H), 6.52 (dd, 1H), 4.16/4.11 (dd+dd, 2H), 3.02/2.55 (dd+dd, 2H), 2.40-1.37 (m, 8H), 2.18 (m, 1H), 2.06 (m, 1H), 1.47/1.37 (m+m, 2H), 1.06 (d, 3H). HRMS calculated for C₃₂H₃₂N₂O₃FSCl: 578.1806; found: 579.1867 (M+H).

### Example 738 (1r,2′R,4R)-4-(3-chloroanilino)-5′-fluoro-2′-{(2R)-2-methyl-3-[(thieno[3,2-b]pyridin-7-yl)oxy]propyl}-2′,3′-dihydrospiro[cyclohexane-1,1′-indene]-4-carboxylic acid

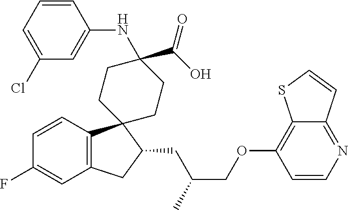 [SMILES: C[C@@H](COc1ccnc2ccsc12)C[C@@H]1Cc2cc(F)ccc2[C@]12CC[C@@](Nc1cccc(Cl)c1)(C(=O)O)CC2]

Using General procedure 32 and Example 737E as the appropriate indane and thieno[3,2-b]pyridin-7-ol as the appropriate alcohol Example 738 was obtained. ¹H NMR (500 MHz, DMSO-d₆) δ ppm: 12.72 (br s, 1H), 8.50 (d, 1H), 7.91 (d, 1H), 7.48 (d, 1H), 7.38 (dd, 1H), 7.03 (m, 1H), 7.02 (t, 1H), 7.00 (d, 1H), 6.96 (m, 1H), 6.59 (t, 1H), 6.53 (br d, 1H), 6.50 (dd, 1H), 6.10 (br s, 1H), 4.24/4.13 (dd+dd, 2H), 3.05/2.61 (dd+dd, 2H), 2.37-1.31 (m, 8H), 2.21 (m, 1H), 2.13 (m, 1H), 1.69/1.29 (m+m, 2H), 1.10 (d, 3H). HRMS calculated for C₃₂H₃₂N₂O₃FSCl: 578.1806; found: 579.1866 (M+H).

### Example 739

### Example 739A methyl (1s,4s)-2′-bromo-4′-chloro-4-(3-chloroanilino)spiro[cyclohexane-1,1′-indene]-4-carboxylate

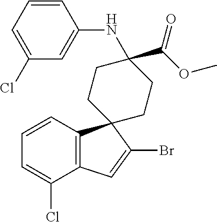 [SMILES: COC(=O)[C@]1(Nc2cccc(Cl)c2)CC[C@]2(CC1)C(Br)=Cc1c(Cl)cccc12]

Using General procedure 17b and Example 137 as the appropriate amino acid, Example 739A was obtained. ¹H NMR (500 MHz, DMSO-d₆) δ ppm: 7.68 (d, 1H), 7.38 (d, 1H), 7.28 (t, 1H), 7.10 (t, 1H), 7.09 (s, 1H), 6.61 (t, 1H), 6.60 (dm, 1H), 6.51 (s, 1H), 6.48 (dm, 1H), 3.68 (s, 3H), 2.37+2.28 (m+m, 4H), 2.21+1.04 (m+m, 4H).

### Example 739B methyl (1r,4R)-4′-chloro-4-(3-chloroanilino)-2′-{(2R)-3-[(4-methoxyphenyl)methoxy]-2-methylpropyl}spiro[cyclohexane-1,1′-indene]-4-carboxylate

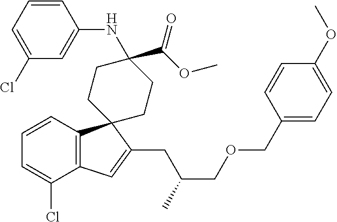 [SMILES: COc1ccc(COC[C@H](C)CC2=Cc3c(Cl)cccc3[C@]23CC[C@@](Nc2cccc(Cl)c2)(C(=O)OC)CC3)cc1]

Using General procedure 27b and Example 739A as the appropriate 2-bromo-indene derivative and Preparation 3d as the appropriate Zn reagent, Example 739B was obtained. ¹H NMR (500 MHz, DMSO-d₆) δ ppm: 7.60 (d, 1H), 7.29 (d, 1H), 7.21 (m, 2H), 7.15 (t, 1H), 7.09 (t, 1H), 6.85 (m, 2H), 6.64 (t, 1H), 6.60 (dm, 1H), 6.52 (t, 1H), 6.48 (dm, 1H), 6.43 (s, 1H), 4.40/4.37 (d+d, 2H), 3.72 (s, 3H), 3.68 (s, 3H), 3.32/3.29 (dd+dd, 2H), 2.43-0.82 (m, 8H), 2.38/2.02 (m+m, 2H), 2.19 (m, 1H), 0.94 (d, 3H). HRMS calculated for C₃₄H₃₇NO₄Cl₂: 593.2100; found: 594.2164 (M+H).

### Example 739C methyl (1r,4R)-4′-chloro-4-(3-chloroanilino)-2′-[(2R)-3-hydroxy-2-methylpropyl]spiro[cyclohexane-1,1′-indene]-4-carboxylate

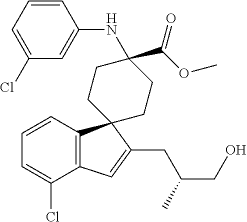 [SMILES: COC(=O)[C@]1(Nc2cccc(Cl)c2)CC[C@]2(CC1)C(C[C@@H](C)CO)=Cc1c(Cl)cccc12]

Using General procedure 28b and Example 739B as the appropriate PMB derivative, Example 739C was obtained. ¹H NMR (500 MHz, DMSO-d₆) δ ppm: 7.60 (d, 1H), 7.30 (d, 1H), 7.15 (t, 1H), 7.10 (t, 1H), 6.63 (dd, 1H), 6.60 (dd, 1H), 6.52 (s, 1H), 6.48 (dd, 1H), 6.43 (s, 1H), 4.57 (t, 1H), 3.68 (s, 3H), 3.35/3.30 (m+m, 2H), 2.41-0.88 (m, 8H), 2.38/1.95 (dd+dd, 2H), 1.98 (m, 1H), 0.90 (d, 3H).). HRMS calculated for C₂₆H₂₉NO₃Cl₂: 473.1524; found: 474.1595 (M+H).

### Example 739 (1r,4R)-4′-chloro-4-(3-chloroanilino)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

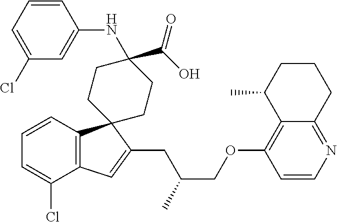 [SMILES: C[C@@H](COc1ccnc2c1[C@H](C)CCC2)CC1=Cc2c(Cl)cccc2[C@]12CC[C@@](Nc1cccc(Cl)c1)(C(=O)O)CC2]

Using General procedure 32 and Example 739C as the appropriate indene and Preparation 2a1 as the appropriate alcohol, Example 739 was obtained. ¹H NMR (500 MHz, DMSO-d₆) δ ppm: 12.85 (br s, 1H), 8.14 (d, 1H), 7.61 (d, 1H), 7.30 (d, 1H), 7.17 (t, 1H), 7.07 (t, 1H), 6.78 (d, 1H), 6.63 (t, 1H), 6.60 (s, 1H), 6.57 (dm, 1H), 6.55 (dm, 1H), 6.36 (br s, 1H), 4.02/3.95 (dd+dd, 2H), 3.06 (m, 1H), 2.75/2.63 (m+m, 2H), 2.51/2.24 (m+m, 2H), 2.49 (m, 1H), 2.42-0.80 (m, 12H), 1.114 (d, 3H), 1.111 (d, 3H). HRMS calculated for C₃₅H₃₈N₂O₃Cl₂: 604.2260; found: 605.2325 (M+H).

### Example 740 and Example 741

### Example 740A methyl (1r,4R)-4′-chloro-4-(3-chloroanilino)-2′-[(2R)-3-hydroxy-2-methylpropyl]-2′,3′-dihydrospiro[cyclohexane-1,1′-indene]-4-carboxylate, diastereoisomer 1

and

### Example 740B methyl (1r,4R)-4′-chloro-4-(3-chloroanilino)-2′-[(2R)-3-hydroxy-2-methylpropyl]-2′,3′-dihydrospiro[cyclohexane-1,1′-indene]-4-carboxylate, diastereoisomer 2

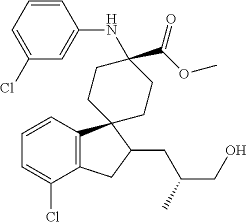

Using General procedure 19 and Example 739B as the appropriate indene, a mixture of diastereoisomers was obtained. The diastereoisomers were separated by chiral chromatography. Column: (R, R) WHELK 0-1, 50×500 mm, 10 μm, Eluents: EtOH/heptane. The diastereoisomer eluting earlier was collected as Example 740A. ¹H NMR (500 MHz, DMSO-d₆) δ ppm: 7.34 (dd, 1H), 7.22 (dd, 1H), 7.21 (t, 1H), 7.07 (t, 1H), 6.59 (t, 1H), 6.56 (dd, 1H), 6.45 (dd, 1H), 6.30 (s, 1H), 4.41 (t, 1H), 3.63 (s, 3H), 3.41/3.20 (m+m, 2H), 3.03/2.55 (dd+dd, 2H), 2.40-1.31 (m, 8H), 2.14 (m, 1H), 1.61 (m, 1H), 1.46/1.05 (m+m, 2H), 0.91 (d, 3H). HRMS calculated for C₂₆H₃₁N₂O₃Cl₂: 475.1681; found: 476.1750 (M+H).

The diastereoisomer eluting later was collected as Example 740B. ¹H NMR (500 MHz, DMSO-d₆) δ ppm: 7.33 (dd, 1H), 7.22 (dd, 1H), 7.21 (t, 1H), 7.06 (t, 1H), 6.58 (t, 1H), 6.57 (dd, 1H), 6.45 (dd, 1H), 6.32 (s, 1H), 4.47 (t, 1H), 3.64 (s, 3H), 3.23 (m, 2H), 3.00/2.51 (dd+dd, 2H), 2.42-1.36 (m, 8H), 2.14 (m, 1H), 1.57 (m, 1H), 1.36/1.08 (m+m, 2H), 0.86 (d, 3H). HRMS calculated for C₂₆H₃₁N₂O₃Cl₂: 475.1681; found: 476.1752 (M+H).

### Example 740 (1r,4R)-4′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 741 (1r,4R)-4′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

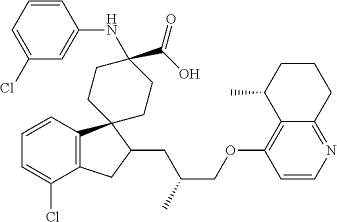

Using General procedure 32 and Example 740A as the appropriate indene and Preparation 2a1 as the appropriate alcohol, Example 740 was obtained. ¹H NMR (400 MHz, DMSO-d₆) δ ppm: 12.73 (br s, 1H), 8.13 (d, 1H), 7.37 (d, 1H), 7.23 (d, 1H), 7.21 (t, 1H), 7.03 (t, 1H), 6.78 (d, 1H), 6.60 (t, 1H), 6.54 (dm, 1H), 6.50 (dm, 1H), 6.15 (br s, 1H), 4.00/3.9 (dd+dd, 2H), 3.07/2.59 (dd+dd, 2H), 3.00 (m, 1H), 2.73/2.60 (m+m, 2H), 2.45-1.26 (m, 12H), 2.14 (m, 1H), 2.09 (m, 1H), 1.71/1.27 (m+m, 2H), 1.11 (d, 3H), 1.07 (d, 3H). HRMS calculated for C₃₅H₄₀N₂O₃Cl₂: 606.2416; found: 607.2496 (M+H).

Using General procedure 32 and Example 740B as the appropriate indene and Preparation 2a1 as the appropriate alcohol Example 741 was obtained. ¹H NMR (400 MHz, DMSO-d₆) δ ppm: 12.73 (br s, 1H), 8.14 (d, 1H), 7.31 (d, 1H), 7.24 (d, 1H), 7.22 (t, 1H), 7.03 (t, 1H), 6.76 (d, 1H), 6.61 (t, 1H), 6.54 (m, 2H), 6.28 (br s, 1H), 3.88 (d, 2H), 3.06/2.57 (dd+dd, 2H), 3.04 (m, 1H), 2.75/2.65 (m+m, 2H), 2.47-1.39 (m, 12H), 2.22 (m, 1H), 2.01 (m, 1H), 1.49/1.34 (m+m, 2H), 1.05 (d, 3H), 0.98 (d, 3H). HRMS calculated for C₃₅H₄₀N₂O₃Cl₂: 606.2416; found: 607.2485 (M+H).

### Example 742 and Example 743

### Example 742A 2-bromo-4-fluoro-2,3-dihydro-1H-inden-1-one

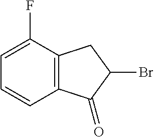 [SMILES: O=C1c2cccc(F)c2CC1Br]

Using General procedure 5 and 4-fluoro-2,3-dihydro-1H-inden-1-one as the appropriate indan-1-one, Example 742A was obtained. MS (EI, 70 eV) m/z (% relative intensity, [ion]): 74 (21), 75 (28), 101 (100), 121 (50), 149 (78), 228 (23, [M+]), 250 (23, [M+]).

### Example 742B 2-bromo-4-fluoro-2,3-dihydro-1H-inden-1-ol

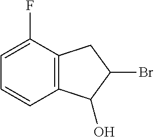 [SMILES: OC1c2cccc(F)c2CC1Br]

Using General procedure 6 and Example 742A as the appropriate bromo-indan-1-one, Example 742B was obtained as a single diastereoisomer. ¹H NMR (500 MHz, DMSO-d₆) δ ppm: 7.31 (m, 1H), 7.17 (d, 1H), 7.07 (t, 1H), 5.96 (d, 1H), 4.99 (m, 1H), 4.92 (m, 1H), 3.45 (dd, 1H), 3.23 (dd, 1H).

### Example 742C 2-bromo-7-fluoro-1H-indene

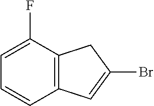 [SMILES: Fc1cccc2c1CC(Br)=C2]

Using General procedure 7 and Example 742B as the appropriate indane, Example 742C was obtained. ¹H NMR (500 MHz, DMSO-d₆) δ ppm: 7.32 (m, 1H), 7.23 (d, 1H), 7.17 (m, 1H), 7.01 (t, 1H), 3.80 (s, 2H).

### Example 742D 2″-bromo-7″-fluorodispiro[[1,3]dioxolane-2,1′-cyclohexane-4′,1″-indene]

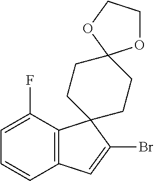 [SMILES: Fc1cccc2c1C1(CCC3(CC1)OCCO3)C(Br)=C2]

and

### Example 742E 2″-bromo-4″-fluorodispiro[[1,3]dioxolane-2,1′-cyclohexane-4′,1″-indene]

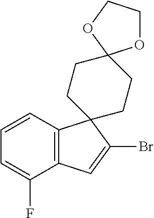 [SMILES: Fc1cccc2c1C=C(Br)C21CCC2(CC1)OCCO2]

Using General procedure 8a and Example 742C as the appropriate indene, a mixture of regioisomers was obtained. The regioisomers were separated via flash chromatography using hexane and EtOAc as eluents. The regioisomer eluting earlier was collected as Example 742D. MS (EI, 70 eV) m/z (% relative intensity, [ion]): 79 (12), 86 (100), 99 (78), 224 (12), 226 (12), 231 (26), 310 (13), 312 (13), 338 (6, [M+]), 340 (6, [M+]).

The regioisomer eluting later was collected as Example 742E. ¹H NMR (500 MHz, DMSO-d₆) δ ppm: 7.54 (d, 1H), 7.26 (m, 1H), 7.14 (t, 1H), 7.13 (s, 1H), 4.00-3.91 (m, 4H), 2.13 (td, 2H), 2.08 (td, 2H), 1.88 (br d, 2H), 1.22 (br d, 2H). MS (EI, 70 eV) m/z (% relative intensity, [ion]): 79 (16), 86 (100), 99 (58), 224 (15), 226 (15), 231 (29), 310 (13), 312 (13), 338 (8, [M+]), 340 (8, [M+]).

### Example 742F 2′-bromo-4′-fluorospiro[cyclohexane-1,1′-inden]-4-one

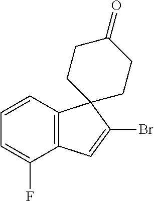 [SMILES: O=C1CCC2(CC1)C(Br)=Cc1c(F)cccc12]

Using General procedure 9 and Example 742E as the appropriate ketal, Example 742F was obtained. ¹H NMR (500 MHz, DMSO-d₆) δ ppm: 7.78 (d, 1H), 7.28 (m, 1H), 7.20 (s, 1H), 7.18 (t, 1H), 2.90 (m, 2H), 2.51 (m, 2H), 2.23 (td, 2H), 1.63 (m, 2H).

### Example 742G 2″-bromo-4″-fluorodispiro[imidazolidine-4,1′-cyclohexane-4′,1″-indene]-2,5-dione

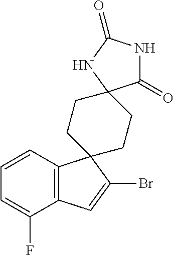 [SMILES: O=C1NC(=O)C2(CCC3(CC2)C(Br)=Cc2c(F)cccc23)N1]

Using General procedure 14 and Example 742F as the appropriate ketone, Example 742G was obtained as a mixture of diastereoisomers. ¹H NMR (500 MHz, DMSO-d₆) δ ppm: 10.69 (br, 1H), 8.89/8.40 (s/s, 1H), 7.74/7.56 (d/d, 1H), 7.33/7.27 (m/m, 1H), 7.21-7.09 (m, 2H), 2.42-1.14 (m, 8H).

### Example 742H 4-amino-2′-bromo-4′-fluorospiro[cyclohexane-1,1′-indene]-4-carboxylic acid

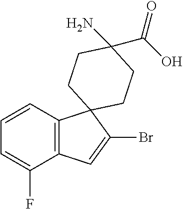 [SMILES: NC1(C(=O)O)CCC2(CC1)C(Br)=Cc1c(F)cccc12]

Using General procedure 15 and Example 742G as the appropriate hydantoin, Example 742H was obtained as a mixture of diastereoisomers. LRMS calculated for C₁₅H₁₅NO₂BrF: 339.0; found: 339.9 (M+H).

### Example 742I 2′-bromo-4′-fluoro-4-(3-chloroanilino)spiro[cyclohexane-1,1′-indene]-4-carboxylic acid

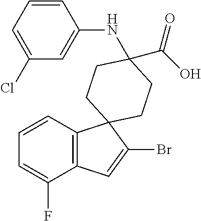 [SMILES: O=C(O)C1(Nc2cccc(Cl)c2)CCC2(CC1)C(Br)=Cc1c(F)cccc12]

Using General procedure 16 and Example 742H as the appropriate amino acid and 1-chloro-3-iodo-benzene as the appropriate iodobenzene, Example 742I was obtained as a mixture of diastereoisomers. ¹H NMR (500 MHz, DMSO-d₆) δ ppm: 12.82 (br s, 1H), 7.56/7.54 (d/d, 1H), 7.35-7.25 (m, 1H), 7.20-7.06 (m, 3H), 6.73-6.53 (m, 3H), 6.40 (br, 1H), 2.66-1.94 (m, 6H), 1.44/1.03 (m/d, 2H).

### Example 742J methyl 2′-bromo-4-(3-chloroanilino)-4′-fluorospiro[cyclohexane-1,1′-indene]-4-carboxylate

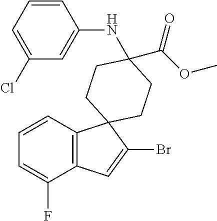 [SMILES: COC(=O)C1(Nc2cccc(Cl)c2)CCC2(CC1)C(Br)=Cc1c(F)cccc12]

Using General procedure 17b and Example 742I as the appropriate amino acid, Example 742J was obtained as a mixture of diastereoisomers. ¹H NMR (500 MHz, DMSO-d₆) δ ppm: 7.56 (m, 1H), 7.30 (m, 1H), 7.16 (m, 1H), 7.13 (s, 1H), 7.10 (t, 1H), 6.62 (t, 1H), 6.60 (dm, 1H), 6.52 (s, 1H), 6.48 (dm, 1H), 3.69 (s, 3H), 2.38/2.28 (m+m, 4H), 2.21/1.03 (m+m, 4H). HRMS calculated for C₂₂H₂₀NO₂FClBr: 463.0350; found: 464.0414 (M+H).

### Example 742K methyl 4-(3-chloroanilino)-4′-fluoro-2′-{(2R)-3-[(4-methoxyphenyl)methoxy]-2-methylpropyl}spiro[cyclohexane-1,1′-indene]-4-carboxylate

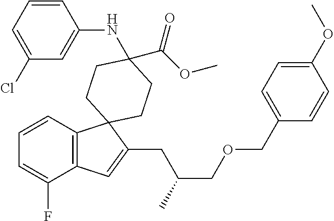 [SMILES: COC(=O)C1(Nc2cccc(Cl)c2)CCC2(CC1)C(C[C@@H](C)COCc1ccc(OC)cc1)=Cc1c(F)cccc12]

Using General procedure 27b and Example 742J as the appropriate 2-bromo-indene derivative and Preparation 3d as the appropriate Zn reagent Example 742K was obtained as a mixture of diastereoisomers. ¹H NMR (500 MHz, DMSO-d₆) δ ppm: 7.49 (dm, 1H), 7.21 (m, 2H), 7.16 (m, 1H), 7.09 (t, 1H), 7.08 (m, 1H), 6.85 (m, 2H), 6.64 (t, 1H), 6.60 (dm, 1H), 6.53 (t, 1H), 6.48 (dm, 1H), 6.44 (s, 1H), 4.40/4.36 (d+d, 2H), 3.72 (s, 3H), 3.68 (s, 3H), 3.32/3.29 (dd+dd, 2H), 2.43-0.83 (m, 11H), 0.93 (d, 3H). HRMS calculated for C₃₄H₃₇NO₄FCl: 577.2395; found: 578.2445 (M+H).

### Example 742L methyl (1r,4R)-4′-fluoro-4-(3-chloroanilino)-2′-[(2R)-3-hydroxy-2-methylpropyl]spiro[cyclohexane-1,1′-indene]-4-carboxylate

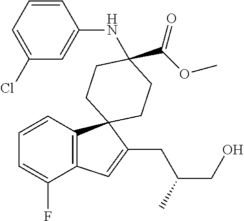 [SMILES: COC(=O)[C@]1(Nc2cccc(Cl)c2)CC[C@]2(CC1)C(C[C@@H](C)CO)=Cc1c(F)cccc12]

Using General procedure 28b and Example 742K as the appropriate PMB derivative Example 742L was isolated as a single diastereoisomer. ¹H NMR (500 MHz, DMSO-d₆) δ ppm: 7.49 (d, 1H), 7.16 (dd, 1H), 7.10 (t, 1H), 7.08 (t, 1H), 6.63 (dd, 1H), 6.59 (dd, 1H), 6.53 (s, 1H), 6.47 (dd, 1H), 6.44 (s, 1H), 3.68 (s, 3H), 3.35/3.30 (m+m, 2H), 2.37/1.92 (dd+dd, 2H), 2.24-0.87 (m, 8H), 2.00 (m, 1H), 0.90 (d, 3H). HRMS calculated for C₂₆H₂₉NO₃FCl: 457.1820; found: 457.1782 (M).

### Example 742M methyl (1r,4R)-4-(3-chloroanilino)-4′-fluoro-2′-[(2R)-3-hydroxy-2-methylpropyl]-2′,3′-dihydrospiro[cyclohexane-1,1′-indene]-4-carboxylate

and

### Example 742N methyl (1r,4R)-4-(3-chloroanilino)-4′-fluoro-2′-[(2R)-3-hydroxy-2-methylpropyl]-2′,3′-dihydrospiro[cyclohexane-1,1′-indene]-4-carboxylate

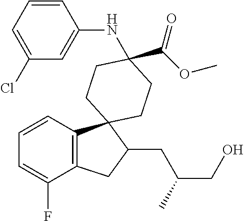

Using General procedure 19 and Example 742L as the appropriate indene a mixture of diastereoisomers was obtained. The diastereoisomers were separated by chiral chromatography. Column: AD, 50×500 mm, 20 μm, Eluents: EtOH/heptane. The diastereoisomer eluting earlier was collected as Example 742M. ¹H NMR (500 MHz, DMSO-d₆) δ ppm: 7.22 (m, 2H), 7.06 (t, 1H), 6.98 (m, 1H), 6.58 (m, 1H), 6.57 (dm, 1H), 6.45 (dm, 1H), 6.30 (s, 1H), 4.39 (t, 1H), 3.64 (s, 3H), 3.40/3.21 (m+m, 2H), 3.02/2.51 (dd+dd, 2H), 2.46-1.25 (m, 8H), 2.15 (m, 1H), 1.62 (m, 1H), 1.47/1.05 (m+m, 2H), 0.91 (d, 3H). HRMS calculated for C₂₆H₃₁NO₃FCl: 459.1977; found: 460.2043 (M+H).

The diastereoisomer eluting later was collected as Example 742N. ¹H NMR (500 MHz, DMSO-d₆) δ ppm: 7.22 (m, 2H), 7.06 (t, 1H), 6.98 (m, 1H), 6.59 (t, 1H), 6.57 (dm, 1H), 6.45 (dm, 1H), 6.32 (s, 1H), 4.46 (t, 1H), 3.64 (s, 3H), 3.22 (m, 2H), 2.98/2.48 (dd+dd, 2H), 2.47-1.31 (m, 8H), 2.14 (m, 1H), 1.57 (m, 1H), 1.37/1.07 (m+m, 2H), 0.85 (d, 3H). HRMS calculated for C₂₆H₃₁NO₃FCl: 459.1977; found: 460.2042 (M+H).

### Example 742 (1r,4R)-4-(3-chloroanilino)-4′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 743 (1r,4R)-4-(3-chloroanilino)-4′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

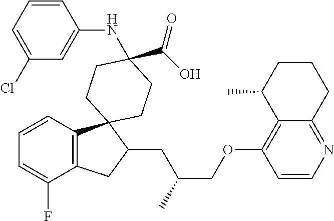

Using General procedure 32 and Example 742M as the appropriate indene and Preparation 2a1 as the appropriate alcohol, Example 742 was obtained. ¹H NMR (500 MHz, DMSO-d₆) δ ppm: 12.72 (br s, 1H), 8.13 (d, 1H), 7.25 (dm, 1H), 7.22 (m, 1H), 7.03 (t, 1H), 6.98 (t, 1H), 6.78 (d, 1H), 6.60 (t, 1H), 6.53 (dm, 1H), 6.50 (dm, 1H), 6.14 (br s, 1H), 4.00/3.89 (dd+dd, 2H), 3.06/2.55 (dd+dd, 2H), 3.00 (m, 1H), 2.73/2.60 (m+m, 2H), 2.44-1.27 (m, 12H), 2.13 (m, 1H), 2.09 (m, 1H), 1.71/1.27 (m+m, 2H), 1.11 (d, 3H), 1.08 (d, 3H). HRMS calculated for C₃₅H₄₀N₂O₃FCl: 590.2712; found: 591.2777 (M+H).

Using General procedure 32 and Example 742N as the appropriate indene and Preparation 2a1 as the appropriate alcohol, Example 743 was obtained. ¹H NMR (500 MHz, DMSO-d₆) δ ppm: 12.7 (br s, 1H), 8.14 (d, 1H), 7.23 (m, 1H), 7.20 (dm, 1H), 7.04 (t, 1H), 6.99 (t, 1H), 6.77 (d, 1H), 6.61 (t, 1H), 6.54 (dm, 1H), 6.53 (dm, 1H), 6.27 (br s, 1H), 3.89/3.87 (dd+dd, 2H), 3.05/2.52 (dd+dd, 2H), 3.04 (m, 1H), 2.76/2.65 (m+m, 2H), 2.47-1.35 (m, 12H), 2.21 (m, 1H), 2.01 (m, 1H), 1.49/1.34 (m+m, 2H), 1.05 (d, 3H), 1.02 (d, 3H). HRMS calculated for C₃₅H₄₀N₂O₃FCl: 590.2712; found: 591.2780 (M+H).

### Example 744

### Example 744A 2-bromo-6,7-difluoro-2,3-dihydro-1H-inden-1-one

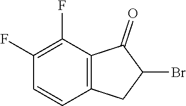 [SMILES: O=C1c2c(ccc(F)c2F)CC1Br]

Using General procedure 5 and 4,5-difluoro-2,3-dihydro-1H-inden-1-one as the appropriate indan-1-one, Example 744A was obtained. MS (EI, 70 eV) m/z (% relative intensity, [ion]): 28 (62), 119 (78), 138 (31), 139 (66), 167 (100), 246 (29, [M+]), 248 (29).

### Example 744B 2-bromo-6,7-difluoro-2,3-dihydro-1H-inden-1-ol

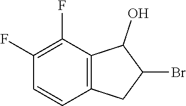 [SMILES: OC1c2c(ccc(F)c2F)CC1Br]

Using General procedure 6 and Example 744A as the appropriate bromo-indan-1-one, Example 744B was obtained. MS (EI, 70 eV) m/z (% relative intensity, [ion]): 55 (41), 75 (33), 101 (34), 119 (31), 151 (88), 169 (100), 248 (29, [M+]), 250 (29, [M+]).

### Example 744C 2-bromo-4,5-difluoro-1H-indene

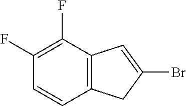 [SMILES: Fc1ccc2c(c1F)C=C(Br)C2]

Using General procedure 7 and Example 744B as the appropriate indane, Example 744C was obtained. ¹H NMR (500 MHz, DMSO-d₆) δ ppm: 7.30-7.17 (m, 3H), 3.81 (s, 2H).

### Example 744D 2″-bromo-4″,5″-difluorodispiro[[1,3]dioxolane-2,1′-cyclohexane-4′,1″-indene]

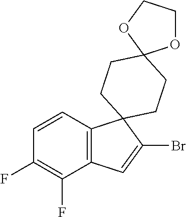 [SMILES: Fc1ccc2c(c1F)C=C(Br)C21CCC2(CC1)OCCO2]

Using General procedure 8a and Example 744C as the appropriate indene, a mixture of regioisomers was obtained. The regioisomers were separated via flash chromatography using hexane and EtOAc as eluents. The regioisomer eluting later was collected as Example 744D. ¹H NMR (500 MHz, DMSO-d₆) δ ppm: 7.51 (dd, 1H), 7.22 (m, 1H), 7.20 (s, 1H), 4.00-3.90 (m, 4H), 2.11 (td, 2H), 2.04 (td, 2H), 1.87 (br d, 2H), 1.21 (br d, 2H).

### Example 744E 2′-bromo-4′,5′-difluorospiro[cyclohexane-1,1′-inden]-4-one

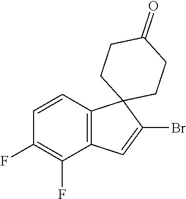 [SMILES: O=C1CCC2(CC1)C(Br)=Cc1c2ccc(F)c1F]

Using General procedure 9 and Example 744D as the appropriate ketal, Example 744E was obtained. ¹H NMR (500 MHz, DMSO-d₆) δ ppm: 7.79 (dd, 1H), 7.28 (s, 1H), 7.26 (ddd, 1H), 2.88/2.50 (m+m, 4H), 2.21/1.62 (m+m, 4H). HRMS calculated for C₁₄H₁₁OF₂Br: 311.9961; found: 311.9950 (M).

### Example 744F 2″-bromo-4″,5″-difluorodispiro[imidazolidine-4,1′-cyclohexane-4′,1″-indene]-2,5-dione

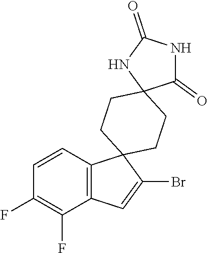 [SMILES: O=C1NC(=O)C2(CCC3(CC2)C(Br)=Cc2c3ccc(F)c2F)N1]

Using General procedure 14 and Example 744E as the appropriate ketone, Example 744F was obtained as a mixture of diastereoisomers. ¹H NMR (500 MHz, DMSO-d₆) δ ppm: 10.73 (br, 1H), 8.95/8.40 (s/s, 1H), 7.56/7.49 (dd/dd, 1H), 7.36/7.29 (m/m, 1H), 7.22/7.21 (s/s, 1H), 2.40-1.15 (m, 8H).

### Example 744G 4-amino-2′-bromo-4′,5′-difluorospiro[cyclohexane-1,1′-indene]-4-carboxylic acid

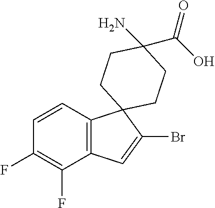 [SMILES: NC1(C(=O)O)CCC2(CC1)C(Br)=Cc1c2ccc(F)c1F]

Using General procedure 15 and Example 744F as the appropriate hydantoin, Example 744G was obtained as a mixture of diastereoisomers. ¹H NMR (500 MHz, DMSO-d₆) δ ppm: 7.85/7.83 (dd/dd, 1H), 7.31/7.23 (m/m, 1H), 7.20/7.16 (s/s, 1H), 2.62-2.46 (m, 2H), 2.20/1.79 (d/d, 2H), 2.06/1.92 (td/td, 2H), 1.11 (d, 2H).

### Example 744H 2′-bromo-4-(3-chloroanilino)-4′,5′-difluorospiro[cyclohexane-1,1′-indene]-4-carboxylic acid

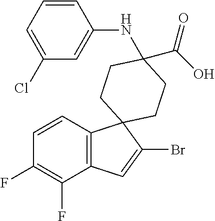 [SMILES: O=C(O)C1(Nc2cccc(Cl)c2)CCC2(CC1)C(Br)=Cc1c2ccc(F)c1F]

Using General procedure 16 and Example 744G as the appropriate amino acid and 1-chloro-3-iodo-benzene as the appropriate iodobenzene, Example 744H was obtained as a mixture of diastereoisomers. LRMS calculated for C₂₁H₁₇NO₂F₂ClBr: 467; found: 468 (M+H).

### Example 7441 methyl (1s,4s)-2′-bromo-4-(3-chloroanilino)-4′,5′-difluorospiro[cyclohexane-1,1′-indene]-4-carboxylate

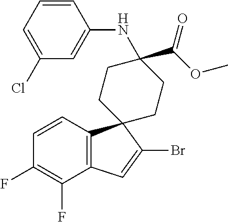 [SMILES: COC(=O)[C@]1(Nc2cccc(Cl)c2)CC[C@]2(CC1)C(Br)=Cc1c(F)c(F)ccc12]

Using General procedure 17b and Example 744H as the appropriate amino acid, a mixture of diastereoisomers was obtained. The diastereoisomers were suspended in hexane and filtered. The solid material was collected as Example 7441. ¹H NMR (500 MHz, DMSO-d₆) δ ppm: 7.52 (dd, 1H), 7.31 (m, 1H), 7.21 (s, 1H), 7.10 (t, 1H), 6.61 (t, 1H), 6.60 (dm, 1H), 6.51 (s, 1H), 6.48 (dm, 1H), 3.68 (s, 3H), 2.33/2.28 (m+m, 4H), 2.2/1.03 (m+m, 4H). HRMS calculated for C₂₂H₁₉NO₂F₂ClBr: 481.0256; found: 482.0325 (M+H).

### Example 744J methyl (1r,4R)-4-(3-chloroanilino)-4′,5′-difluoro-2′-{(2R)-3-[(4-methoxyphenyl)methoxy]-2-methylpropyl}spiro[cyclohexane-1,1′-indene]-4-carboxylate

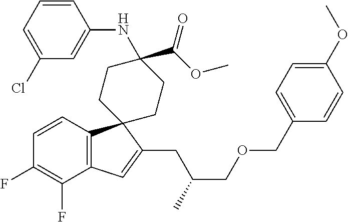 [SMILES: COc1ccc(COC[C@H](C)CC2=Cc3c(F)c(F)ccc3[C@]23CC[C@@](Nc2cccc(Cl)c2)(C(=O)OC)CC3)cc1]

Using General procedure 27b and Example 7441 as the appropriate 2-bromo-indene derivative and Preparation 3d as the appropriate Zn reagent, Example 744J was obtained. ¹H NMR (500 MHz, DMSO-d₆) δ ppm: 7.42 (dd, 1H), 7.21 (m, 2H), 7.15 (m, 1H), 7.09 (t, 1H), 6.85 (m, 2H), 6.63 (t, 1H), 6.60 (dm, 1H), 6.57 (s, 1H), 6.47 (dm, 1H), 6.43 (s, 1H), 4.39/4.36 (d+d, 2H), 3.72 (s, 3H), 3.68 (s, 3H), 3.32/3.29 (dd+dd, 2H), 2.39/2.01 (dd+dd, 2H), 2.38-0.84 (m, 8H), 2.20 (m, 1H), 0.93 (d, 3H). HRMS calculated for C₃₄H₃₆NO₄F₂Cl: 595.2301; found: 596.2376 (M+H).

### Example 744K methyl (1r,4R)-4-(3-chloroanilino)-4′,5′-difluoro-2′-[(2R)-3-hydroxy-2-methylpropyl]spiro[cyclohexane-1,1′-indene]-4-carboxylate

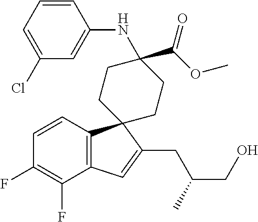 [SMILES: COC(=O)[C@]1(Nc2cccc(Cl)c2)CC[C@]2(CC1)C(C[C@@H](C)CO)=Cc1c(F)c(F)ccc12]

Using General procedure 28b and Example 744J as the appropriate PMB derivative, Example 744K was obtained. ¹H NMR (500 MHz, DMSO-d₆) δ ppm: 7.42 (dd, 1H), 7.15 (m, 1H), 7.09 (t, 1H), 6.62 (t, 1H), 6.60 (dm, 1H), 6.57 (s, 1H), 6.47 (dm, 1H), 6.43 (s, 1H), 4.57 (t, 1H), 3.68 (s, 3H), 3.32 (m, 2H), 2.38/1.95 (dm+m, 2H), 2.37-0.86 (m, 8H), 2.00 (m, 1H), 0.89 (d, 3H). HRMS calculated for C₂₆H₂₈NO₃F₂Cl: 475.1726; found: 476.1794 (M+H).

### Example 744 (1r,4R)-4-(3-chloroanilino)-4′,5′-difluoro-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

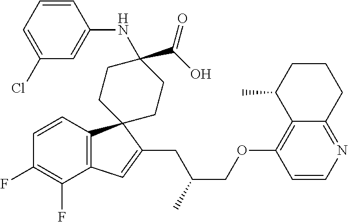 [SMILES: C[C@@H](COc1ccnc2c1[C@H](C)CCC2)CC1=Cc2c(F)c(F)ccc2[C@]12CC[C@@](Nc1cccc(Cl)c1)(C(=O)O)CC2]

Using General procedure 32 and Example 744K as the appropriate indene and Preparation 2a1 as the appropriate alcohol, Example 744 was obtained. ¹H NMR (500 MHz, DMSO-d₆) δ ppm: 12.90 (br s, 1H), 8.13 (d, 1H), 7.44 (dd, 1H), 7.17 (m, 1H), 7.07 (t, 1H), 6.77 (d, 1H), 6.67 (s, 1H), 6.62 (t, 1H), 6.56 (dm, 1H), 6.55 (dm, 1H), 6.36 (br s, 1H), 4.01/3.94 (dd+dd, 2H), 3.05 (m, 1H), 2.75/2.63 (m+m, 2H), 2.51/2.24 (dd+dd, 2H), 2.50 (m, 1H), 2.42-0.78 (m, 12H), 1.10 (d, 3H), 1.10 (d, 3H). HRMS calculated for C₃₅H₃₇N₂O₃F₂Cl: 606.2461; found: 607.2543 (M+H).

### Example 745

### Example 745A 2-bromo-7-chloro-6-fluoro-2,3-dihydro-1H-inden-1-one

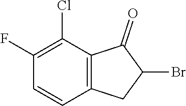 [SMILES: O=C1c2c(ccc(F)c2Cl)CC1Br]

Using General procedure 5 and 4-chloro-5-fluoro-2,3-dihydro-1H-inden-1-one as the appropriate indan-1-one, Example 745A was obtained. MS (EI, 70 eV) m/z (% relative intensity, [ion]): 28 (30), 120 (52), 135 (25), 155 (33), 183 (100), 185 (33), 262 (28, [M+]), 264 (36, [M+]).

### Example 745B 2-bromo-7-chloro-6-fluoro-2,3-dihydro-1H-inden-1-ol

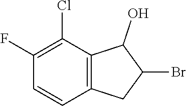 [SMILES: OC1c2c(ccc(F)c2Cl)CC1Br]

Using General procedure 6 and Example 745A as the appropriate bromo-indan-1-one, Example 745B was obtained. ¹H NMR (500 MHz, DMSO-d₆) δ ppm: 7.32 (dd, 1H), 7.25 (dd, 1H), 5.91 (d, 1H), 4.97 (dd, 1H), 4.72 (ddd, 1H), 3.35/3.21 (m+m, 2H). HRMS calculated for C₉H₇₀FClBr: 263.9353; found: 263.9354 (M).

### Example 745C 2-bromo-4-chloro-5-fluoro-1H-indene

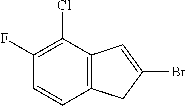 [SMILES: Fc1ccc2c(c1Cl)C=C(Br)C2]

Using General procedure 7 and Example 745B as the appropriate indane, Example 745C was obtained. ¹H NMR (500 MHz, DMSO-d₆) δ ppm: 7.39 (dd, 1H), 7.21 (dd, 1H), 7.19 (s, 1H), 3.85 (s, 2H).

### Example 745D 2″-bromo-4″-chloro-5″-fluorodispiro[[1,3]dioxolane-2,1′-cyclohexane-4′,1″-indene]

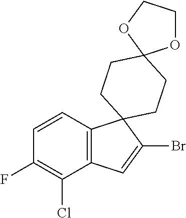 [SMILES: Fc1ccc2c(c1Cl)C=C(Br)C21CCC2(CC1)OCCO2]

Using General procedure 8a and Example 745C as the appropriate indene, a mixture of regioisomers was obtained. The regioisomers were separated via flash chromatography using hexane and EtOAc as eluents. The regioisomer eluting later was collected as Example 745D. ¹H NMR (500 MHz, DMSO-d₆) δ ppm: 7.68 (dd, 1H), 7.21 (dd, 1H), 7.15 (s, 1H), 3.96 (br s, 4H), 2.12 (td, 2H), 2.05 (td, 2H), 1.88 (br d, 2H), 1.23 (br d, 2H).

### Example 745E 2′-bromo-4′-chloro-5′-fluorospiro[cyclohexane-1,1′-inden]-4-one

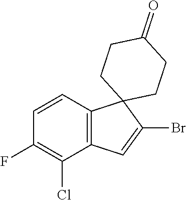 [SMILES: O=C1CCC2(CC1)C(Br)=Cc1c2ccc(F)c1Cl]

Using General procedure 9 and Example 745D as the appropriate ketal, Example 745E was obtained. ¹H NMR (500 MHz, DMSO-d₆) δ ppm: 7.94 (dd, 1H), 7.24 (dd, 1H), 7.22 (s, 1H), 2.88/2.50 (m+m, 4H), 2.21/1.64 (m+m, 4H). HRMS calculated for C₁₄H₁₁OFClBr: 327.9666; found: 327.9652 (M).

### Example 745F 2″-bromo-4″-chloro-5″-fluorodispiro[imidazolidine-4,1′-cyclohexane-4′,1″-indene]-2,5-dione

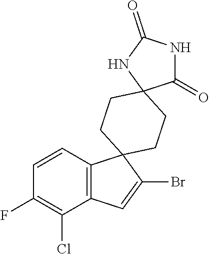 [SMILES: O=C1NC(=O)C2(CCC3(CC2)C(Br)=Cc2c3ccc(F)c2Cl)N1]

Using General procedure 14 and Example 745E as the appropriate ketone Example 745F was obtained as a mixture of diastereoisomers. ¹H NMR (500 MHz, DMSO-d₆) δ ppm: 10.80/10.64 (s, 1H), 8.96/8.42 (s/s, 1H), 7.85/7.63 (dd/dd, 1H), 7.35/7.29 (dd/dd, 1H), 7.17/7.15 (s/s, 1H), 2.35-1.20 (m, 8H).

### Example 745G 4-amino-2′-bromo-4′-chloro-5′-fluorospiro[cyclohexane-1,1′-indene]-4-carboxylic acid

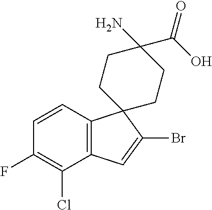 [SMILES: NC1(C(=O)O)CCC2(CC1)C(Br)=Cc1c2ccc(F)c1Cl]

Using General procedure 15 and Example 745F as the appropriate hydantoin, Example 745G was obtained as a mixture of diastereoisomers. ¹H NMR (500 MHz, DMSO-d₆) δ ppm: 8.01/7.63 (dd/dd, 1H), 7.35/7.30 (t/t, 1H), 7.17/7.14 (s/s, 1H), 2.66-1.05 (m, 8H).

### Example 745H 2′-bromo-4′-chloro-4-(3-chloroanilino)-5′-fluorospiro[cyclohexane-1,1′-indene]-4-carboxylic acid

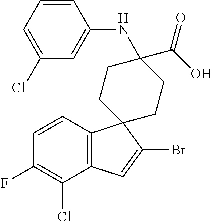 [SMILES: O=C(O)C1(Nc2cccc(Cl)c2)CCC2(CC1)C(Br)=Cc1c2ccc(F)c1Cl]

Using General procedure 16 and Example 745G as the appropriate amino acid and 1-chloro-3-iodo-benzene as the appropriate iodobenzene, Example 745H was obtained as a mixture of diastereoisomers. LRMS calculated for C₂₁H₁₇NO₂FCl₂Br: 483; found: 484 (M+H).

### Example 7451 methyl (1s,4s)-2′-bromo-4′-chloro-4-(3-chloroanilino)-5′-fluorospiro[cyclohexane-1,1′-indene]-4-carboxylate

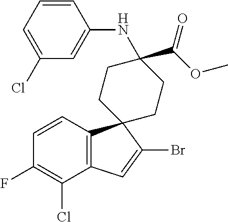 [SMILES: COC(=O)[C@]1(Nc2cccc(Cl)c2)CC[C@]2(CC1)C(Br)=Cc1c(Cl)c(F)ccc12]

Using General procedure 17b and Example 745H as the appropriate amino acid, a mixture of diastereoisomers was obtained. The diastereoisomers were suspended in hexane and filtered, washed with hexane and cold MeOH. The solid material was collected as Example 7451. ¹H NMR (500 MHz, DMSO-d₆) δ ppm: 7.67 (dd, 1H), 7.29 (dd, 1H), 7.15 (s, 1H), 7.10 (t, 1H), 6.61 (dd, 1H), 6.60 (dd, 1H), 6.51 (s, 1H), 6.48 (dd, 1H), 3.68 (s, 3H), 2.33/2.29 (t+d, 4H), 2.20/1.05 (t+d, 4H). HRMS calculated for C₂₂H₁₉NO₂FCl₂Br: 496.9960; found: 498.0031 (M+H).

### Example 745J methyl (1r,4R)-4′-chloro-4-(3-chloroanilino)-5′-fluoro-2′-{(2R)-3-[(4-methoxyphenyl)methoxy]-2-methylpropyl}spiro[cyclohexane-1,1′-indene]-4-carboxylate

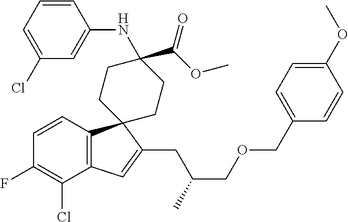 [SMILES: COc1ccc(COC[C@H](C)CC2=Cc3c(Cl)c(F)ccc3[C@]23CC[C@@](Nc2cccc(Cl)c2)(C(=O)OC)CC3)cc1]

Using General procedure 27b and Example 7451 as the appropriate 2-bromo-indene derivative and Preparation 3d as the appropriate Zn reagent, Example 745J was obtained. ¹H NMR (500 MHz, DMSO-d₆) δ ppm: 7.57 (dd, 1H), 7.21 (d, 2H), 7.14 (dd, 1H), 7.09 (t, 1H), 6.85 (d, 2H), 6.64 (t, 1H), 6.60 (dd, 1H), 6.54 (s, 1H), 6.48 (dd, 1H), 6.43 (s, 1H), 4.39/4.36 (d+d, 2H), 3.72 (s, 3H), 3.68 (s, 3H), 3.32/3.29 (dd+dd, 2H), 2.40/2.04 (dd+dd, 2H), 2.31/2.21 (t+m, 4H), 2.19 (m, 1H), 2.11/0.9 (t+d, 4H), 0.93 (d, 3H). HRMS calculated for C₃₄H₃₆NO₄FCl₂: 611.2006; found: 612.2062 (M+H).

### Example 745K methyl (1r,4R)-4′-chloro-4-(3-chloroanilino)-5′-fluoro-2′-[(2R)-3-hydroxy-2-methylpropyl]spiro[cyclohexane-1,1′-indene]-4-carboxylate

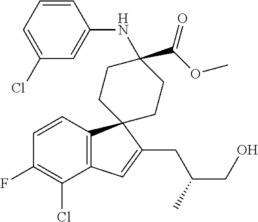 [SMILES: COC(=O)[C@]1(Nc2cccc(Cl)c2)CC[C@]2(CC1)C(C[C@@H](C)CO)=Cc1c(Cl)c(F)ccc12]

Using General procedure 28b and Example 745J as the appropriate PMB derivative, Example 745K was obtained. ¹H NMR (500 MHz, DMSO-d₆) δ ppm: 7.58 (dd, 1H), 7.14 (dd, 1H), 7.10 (t, 1H), 6.63 (t, 1H), 6.60 (dm, 1H), 6.55 (s, 1H), 6.47 (dm, 1H), 6.43 (s, 1H), 4.58 (t, 1H), 3.68 (s, 3H), 3.34/3.31 (m+m, 2H), 2.41/1.96 (dd+dd, 2H), 2.39-0.86 (m, 8H), 2.00 (m, 1H), 0.90 (d, 3H). HRMS calculated for C₂₆H₂₈NO₃FCl₂: 491.1430; found: 492.1505 (M+H).

### Example 745 (1r,4R)-4′-chloro-4-(3-chloroanilino)-5′-fluoro-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

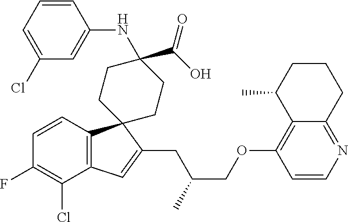 [SMILES: C[C@@H](COc1ccnc2c1[C@H](C)CCC2)CC1=Cc2c(Cl)c(F)ccc2[C@]12CC[C@@](Nc1cccc(Cl)c1)(C(=O)O)CC2]

Using General procedure 32 and Example 745K as the appropriate indene and Preparation 2a1 as the appropriate alcohol, Example 745 was obtained. ¹H NMR (500 MHz, DMSO-d₆) δ ppm: 12.94 (br s, 1H), 8.14 (d, 1H), 7.59 (dd, 1H), 7.16 (dd, 1H), 7.07 (t, 1H), 6.78 (d, 1H), 6.63 (s, 1H), 6.62 (t, 1H), 6.56 (dd, 1H), 6.55 (dd, 1H), 6.35 (br s, 1H), 4.02/3.95 (dd+dd, 2H), 3.05 (m, 1H), 2.75/2.63 (dd+dd, 2H), 2.57-0.85 (m, 8H), 2.53/2.25 (dd+dd, 2H), 2.49 (m, 1H), 1.78/1.69 (m+m, 2H), 1.58/1.53 (m+m, 2H), 1.11 (d, 3H), 1.10 (d, 3H). HRMS calculated for C₃₅H₃₇N₂O₃FCl₂: 622.2166; found: 623.2241 (M+H).

### Example 746 and Example 747

### Example 746A methyl (1r,4R)-4′-chloro-4-(3-chloroanilino)-5′-fluoro-2′-[(2R)-3-hydroxy-2-methylpropyl]-2′,3′-dihydrospiro[cyclohexane-1,1′-indene]-4-carboxylate, diastereoisomer 1

and

### Example 746B methyl (1r,4R)-4′-chloro-4-(3-chloroanilino)-5′-fluoro-2′-[(2R)-3-hydroxy-2-methylpropyl]-2′,3′-dihydrospiro[cyclohexane-1,1′-indene]-4-carboxylate, diastereoisomer 2

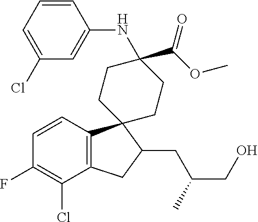

Using General procedure 19 and Example 745K as the appropriate indene, a mixture of diastereoisomers was obtained. The diastereoisomers were separated by chiral chromatography. Column: IC, 50×500 mm, 20 μm, Eluents: nPrOH/heptane. The diastereoisomer eluting earlier was collected as Example 746A. ¹H NMR (500 MHz, DMSO-d₆) δ ppm: 7.35 (dd, 1H), 7.20 (t, 1H), 7.06 (t, 1H), 6.58 (t, 1H), 6.57 (dd, 1H), 6.44 (dd, 1H), 6.30 (s, 1H), 4.41 (t, 1H), 3.64 (s, 3H), 3.40/3.21 (dt+dt, 2H), 3.06/2.59 (dd+dd, 2H), 2.42-1.02 (m, 8H), 2.19 (m, 1H), 1.62 (m, 1H), 1.46/1.06 (dd+t, 2H), 0.91 (d, 3H). HRMS calculated for C₂₆H₃₀NO₃FCl₂: 493.1587; found: 494.1660 (M+H).

The diastereoisomer eluting later was collected as Example 746B. ¹H NMR (500 MHz, DMSO-d₆) δ ppm: 7.34 (dd, 1H), 7.21 (t, 1H), 7.06 (t, 1H), 6.58 (t, 1H), 6.57 (dd, 1H), 6.44 (dd, 1H), 6.31 (s, 1H), 4.47 (t, 1H), 3.64 (s, 3H), 3.25/3.21 (dt+dt, 2H), 3.02/2.56 (dd+dd, 2H), 2.43-1.04 (m, 8H), 2.17 (m, 1H), 1.56 (m, 1H), 1.38/1.07 (dd+t, 2H), 0.86 (d, 3H). HRMS calculated for C₂₆H₃₀NO₃FCl₂: 493.1587; found: 494.1660 (M+H).

### Example 746 (1r,4R)-4′-chloro-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 747 (1r,4R)-4′-chloro-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

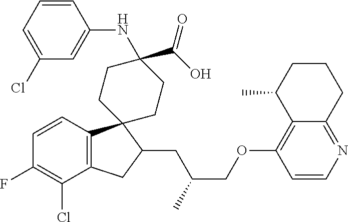

Using General procedure 32 and Example 746A as the appropriate indane and Preparation 2a1 as the appropriate alcohol, Example 746 was obtained. ¹H NMR (500 MHz, DMSO-d₆) δ ppm: 8.13 (d, 1H), 7.35 (dd, 1H), 7.16 (t, 1H), 7.01 (t, 1H), 6.76 (d, 1H), 6.65 (m, 1H), 6.54 (br s, 1H), 6.53 (br s, 1H), 4.02/3.91 (dd+dd, 2H), 3.11/2.64 (d+d, 2H), 3.05 (m, 1H), 2.75/2.63 (d+m, 2H), 2.42-1.34 (m, 8H), 2.23 (m, 1H), 2.10 (m, 1H), 1.79/1.69 (m/m, 2H), 1.74/1.28 (m/m, 2H), 1.59/1.50 (m+m, 2H), 1.12 (d, 3H), 1.10 (d, 3H). HRMS calculated for C₃₅H₃₉N₂O₃FCl₂: 624.2322; found: 625.2397 (M+H).

Using General procedure 32 and Example 746B as the appropriate indane and Preparation 2a1 as the appropriate alcohol, Example 747 was obtained. ¹H NMR (500 MHz, DMSO-d₆) δ ppm: 8.14 (d, 1H), 7.30 (dd, 1H), 7.21 (t, 1H), 7.01 (t, 1H), 6.76 (d, 1H), 6.61 (t, 1H), 6.53 (m, 2H), 3.87 (m, 2H), 3.09/2.59 (dd+dd, 2H), 3.02 (m, 1H), 2.75/2.64 (dm+m, 2H), 2.43-1.41 (m, 8H), 2.27 (m, 1H), 2.00 (m, 1H), 1.79/1.73 (m+m, 2H), 1.67/1.58 (m+d, 2H), 1.49/1.32 (m+m, 2H), 1.05 (d, 3H), 0.96 (d, 3H). HRMS calculated for C₃₅H₃₉N₂O₃FCl₂: 624.2322; found: 625.2403 (M+H).

### Example 748 and Example 749

### Example 748A 2-bromo-6-chloro-7-fluoro-2,3-dihydro-1H-inden-1-one

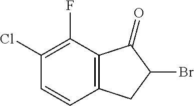 [SMILES: O=C1c2c(ccc(Cl)c2F)CC1Br]

Using General procedure 5 and 6-chloro-7-fluoro-2,3-dihydro-1H-inden-1-one as the appropriate indan-1-one, Example 748A was obtained. ¹H NMR (500 MHz, DMSO-d₆) δ ppm: 7.96 (dd, 1H), 7.44 (d, 1H), 5.06 (dd, 1H), 3.87/3.33 (ddd+ddd, 2H). HRMS calculated for C₉H₅OFClBr: 261.9196; found: 261.9190 (M).

### Example 748B 2-bromo-6-chloro-7-fluoro-2,3-dihydro-1H-inden-1-ol

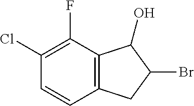 [SMILES: OC1c2c(ccc(Cl)c2F)CC1Br]

Using General procedure 6 and Example 748A as the appropriate bromo-indan-1-one, Example 748B was obtained as a single diastereoisomer. ¹H NMR (500 MHz, DMSO-d₆) δ ppm: 7.47 (t, 1H), 7.12 (d, 1H), 5.97 (d, 1H), 5.13 (dd, 1H), 4.97-4.74 (m, 1H), 3.39 (dd, 1H), 3.21 (dd, 1H).

### Example 748C 2-bromo-5-chloro-4-fluoro-1H-indene

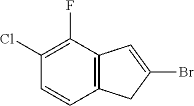 [SMILES: Fc1c(Cl)ccc2c1C=C(Br)C2]

Using General procedure 7 and Example 748B as the appropriate indane, Example 748C was obtained. ¹H NMR (500 MHz, DMSO-d₆) δ ppm: 7.38 (t, 1H), 7.28 (d, 1H), 7.24 (s, 1H), 3.85 (s, 2H).

### Example 748D 2″-bromo-5″-chloro-4″-fluorodispiro[[1,3]dioxolane-2,1′-cyclohexane-4′,1″-indene] and 2″-bromo-6″-chloro-7″-fluorodispiro[[1,3]dioxolane-2,1′-cyclohexane-4′,1″-indene]

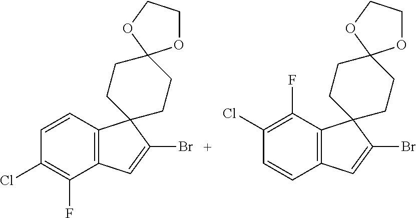 [SMILES: Fc1c(Cl)ccc2c1C=C(Br)C21CCC2(CC1)OCCO2 ; Fc1c(Cl)ccc2c1C1(CCC3(CC1)OCCO3)C(Br)=C2]

Using General procedure 8a and Example 748C as the appropriate indene, Example 748D was obtained as a mixture of regioisomers. MS (EI, 70 eV) m/z (% relative intensity, [ion]): 28 (28), 42 (23), 86 (100), 99 (31), 372 (6, [M+]), 374 (9, [M+]).

### Example 748E 2′-bromo-5′-chloro-4′-fluorospiro[cyclohexane-1,1′-inden]-4-one

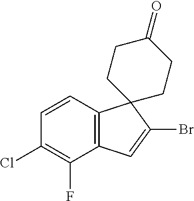 [SMILES: O=C1CCC2(CC1)C(Br)=Cc1c2ccc(Cl)c1F]

Using General procedure 9 and Example 748D as the appropriate ketal, a mixture of regioisomers was obtained. The regioisomers were separated via flash chromatography using hexane and EtOAc as eluents. The regioisomer eluting later was collected as Example 748E. ¹H NMR (500 MHz, DMSO-d₆) δ ppm: 7.80 (d, 1H), 7.41 (t, 1H), 7.25 (s, 1H), 2.87 (ddd, 2H), 2.55-2.46 (m, 2H), 2.22 (td, 2H), 1.65 (dm, 2H).

### Example 748F 2″-bromo-5″-chloro-4″-fluorodispiro[imidazolidine-4,1′-cyclohexane-4′,1″-indene]-2,5-dione

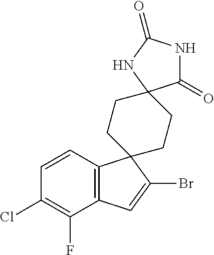 [SMILES: O=C1NC(=O)C2(CCC3(CC2)C(Br)=Cc2c3ccc(Cl)c2F)N1]

Using General procedure 14 and Example 748E as the appropriate ketone Example 748F was obtained as a mixture of diastereoisomers. ¹H NMR (500 MHz, DMSO-d₆) δ ppm: 10.85-10.50 (br s, 1H), 8.95/8.41 (s/s, 1H), 7.77-7.42 (m, 2H), 7.21/7.19 (s/s, 1H), 2.40-1.19 (m, 8H).

### Example 748G 4-amino-2′-bromo-5′-chloro-4′-fluorospiro[cyclohexane-1,1′-indene]-4-carboxylic acid

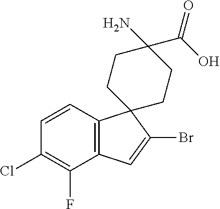 [SMILES: NC1(C(=O)O)CCC2(CC1)C(Br)=Cc1c2ccc(Cl)c1F]

Using General procedure 15 and Example 748F as the appropriate hydantoin, Example 748G was obtained as a mixture of diastereoisomers. ¹H NMR (500 MHz, DMSO-d₆) δ ppm: 7.87 (d, 1H), 7.45/7.38 (t/t, 1H), 7.17/7.14 (s/s, 1H), 2.62-1.03 (m, 8H).

### Example 748H 2′-bromo-5′-chloro-4-(3-chloroanilino)-4′-fluorospiro[cyclohexane-1,1′-indene]-4-carboxylic acid

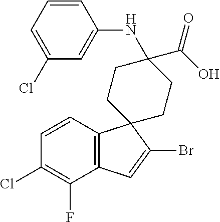 [SMILES: O=C(O)C1(Nc2cccc(Cl)c2)CCC2(CC1)C(Br)=Cc1c2ccc(Cl)c1F]

Using General procedure 16 and Example 748G as the appropriate amino acid and 1-chloro-3-iodo-benzene as the appropriate iodobenzene, Example 748H was obtained as a mixture of diastereoisomers. LRMS calculated for C₂₁H₁₇NO₂FCl₂Br: 483; found: 484 (M+H).

### Example 7481 methyl (1s,4s)-2′-bromo-5′-chloro-4-(3-chloroanilino)-4′-fluorospiro[cyclohexane-1,1′-indene]-4-carboxylate

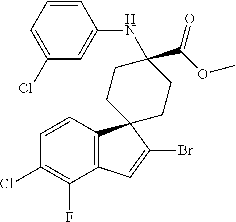 [SMILES: COC(=O)[C@]1(Nc2cccc(Cl)c2)CC[C@]2(CC1)C(Br)=Cc1c(F)c(Cl)ccc12]

Using General procedure 17b and Example 748H as the appropriate amino acid a mixture of diastereoisomers was obtained. The diastereoisomers were suspended in hexane and filtered, washed with hexane, dried. The solid material was collected as Example 7481. ¹H NMR (500 MHz, DMSO-d₆) δ ppm: 7.55 (d, 1H), 7.46 (t, 1H), 7.19 (s, 1H), 7.10 (t, 1H), 6.61 (t, 1H), 6.60 (dd, 1H), 6.51 (s, 1H), 6.48 (dd, 1H), 3.68 (s, 3H), 2.33/2.28 (t+d, 4H), 2.21/1.05 (t+d, 4H). HRMS calculated for C₂₂H₁₉NO₂FCl₂Br: 496.9960; found: 498.0050 (M+H).

### Example 748J methyl (1r,4R)-5′-chloro-4-(3-chloroanilino)-4′-fluoro-2′-{(2R)-3-[(4-methoxyphenyl)methoxy]-2-methylpropyl}spiro[cyclohexane-1,1′-indene]-4-carboxylate

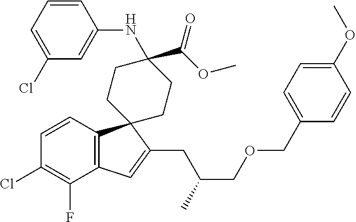 [SMILES: COc1ccc(COC[C@H](C)CC2=Cc3c(F)c(Cl)ccc3[C@]23CC[C@@](Nc2cccc(Cl)c2)(C(=O)OC)CC3)cc1]

Using General procedure 27b and Example 7481 as the appropriate 2-bromo-indene derivative and Preparation 3d as the appropriate Zn reagent, Example 748J was obtained. ¹H NMR (500 MHz, DMSO-d₆) δ ppm: 7.46 (d, 1H), 7.32 (t, 1H), 7.21 (dm, 2H), 7.09 (t, 1H), 6.85 (dm, 2H), 6.64 (t, 1H), 6.60 (dm, 1H), 6.56 (s, 1H), 6.48 (dm, 1H), 6.44 (s, 1H), 4.39/4.36 (d+d, 2H), 3.72 (s, 3H), 3.68 (s, 3H), 3.33/3.29 (dd+dd, 2H), 2.38/2.01 (dd+dd, 2H), 2.36-0.85 (m, 8H), 2.21 (m, 1H), 0.93 (d, 3H). HRMS calculated for C₃₄H₃₆NO₄FCl₂: 611.2006; found: 612.2092 (M+H).

### Example 748K methyl (1r,4R)-5′-chloro-4-(3-chloroanilino)-4′-fluoro-2′-[(2R)-3-hydroxy-2-methylpropyl]spiro[cyclohexane-1,1′-indene]-4-carboxylate

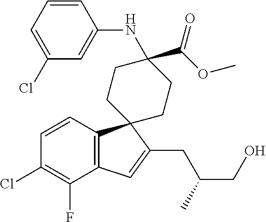 [SMILES: COC(=O)[C@]1(Nc2cccc(Cl)c2)CC[C@]2(CC1)C(C[C@@H](C)CO)=Cc1c(F)c(Cl)ccc12]

Using General procedure 28b and Example 748J as the appropriate PMB derivative, Example 748K was obtained. ¹H NMR (500 MHz, DMSO-d₆) δ ppm: 7.47 (d, 1H), 7.32 (dd, 1H), 7.10 (t, 1H), 6.63 (t, 1H), 6.60 (dm, 1H), 6.56 (s, 1H), 6.47 (dm, 1H), 6.44 (s, 1H), 4.57 (br s, 1H), 3.68 (s, 3H), 3.33/3.30 (dd+dd, 2H), 2.39/1.93 (dd+dd, 2H), 2.36-0.85 (m, 8H), 2.00 (m, 1H), 0.89 (d, 3H). HRMS calculated for C₂₆H₂₈NO₃FCl₂: 491.1430; found: 492.1498 (M+H).

### Example 748L methyl (1r,4R)-5′-chloro-4-(3-chloroanilino)-4′-fluoro-2′-[(2R)-3-hydroxy-2-methylpropyl]-2′,3′-dihydrospiro[cyclohexane-1,1′-indene]-4-carboxylate, diastereoisomer 1

and

### Example 748M methyl (1r,4R)-5′-chloro-4-(3-chloroanilino)-4′-fluoro-2′-[(2R)-3-hydroxy-2-methylpropyl]-2′,3′-dihydrospiro[cyclohexane-1,1′-indene]-4-carboxylate, diastereoisomer 1

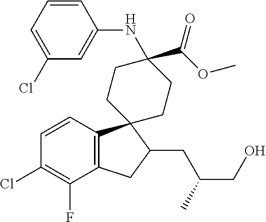

Using General procedure 19 and Example 748K as the appropriate indene, a mixture of diastereoisomers was obtained. The diastereoisomers were separated by chiral chromatography. Column: IC, 50×500 mm, 20 μm, Eluents: nPrOH/heptane. The diastereoisomer eluting earlier was collected as Example 748L. ¹H NMR (500 MHz, DMSO-d₆) δ ppm: 7.38 (t, 1H), 7.22 (d, 1H), 7.06 (t, 1H), 6.58 (t, 1H), 6.57 (dm, 1H), 6.44 (dm, 1H), 6.03 (s, 1H), 4.40 (t, 1H), 3.63 (s, 3H), 3.39/3.22 (m+m, 2H), 3.06/2.57 (dd+dd, 2H), 2.43-1.28 (m, 8H), 2.19 (m, 1H), 1.62 (m, 1H), 1.46/1.05 (m+m, 2H), 0.91 (d, 3H). HRMS calculated for C₂₆H₃₀NO₃FCl₂: 493.1587; found: 494.1665 (M+H).

The diastereoisomer eluting earlier was collected as Example 748M. ¹H NMR (500 MHz, DMSO-d₆) δ ppm: 7.38 (t, 1H), 7.22 (d, 1H), 7.06 (t, 1H), 6.58 (t, 1H), 6.57 (dm, 1H), 6.44 (dm, 1H), 6.32 (s, 1H), 4.46 (t, 1H), 3.64 (s, 3H), 3.22 (m, 2H), 3.03/2.53 (dd+dd, 2H), 2.44-1.31 (m, 8H), 2.18 (m, 1H), 1.56 (m, 1H), 1.37/1.07 (m+m, 2H), 0.85 (d, 3H). HRMS calculated for C₂₆H₃₀NO₃FCl₂: 493.1587; found: 494.1659 (M+H).

### Example 748 (1r,4R)-5′-chloro-4-(3-chloroanilino)-4′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 749 (1r,4R)-5′-chloro-4-(3-chloroanilino)-4′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

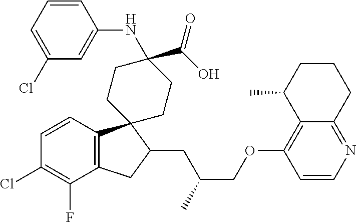

Using General procedure 32 and Example 748L as the appropriate indene and Preparation 2a1 as the appropriate alcohol, Example 748 was obtained. ¹H NMR (500 MHz, DMSO-d₆) δ ppm: 12.77 (br s, 1H), 8.13 (d, 1H), 7.38 (t, 1H), 7.24 (d, 1H), 7.03 (t, 1H), 6.77 (d, 1H), 6.60 (t, 1H), 6.53 (dd, 1H), 6.49 (dd, 1H), 6.15 (br s, 1H), 4.89/4.00 (dd+dd, 2H), 3.11/2.61 (dd+dd, 2H), 2.99 (m, 1H), 2.73/2.59 (dm+m, 2H), 2.41-1.29 (m, 8H), 2.17 (m, 1H), 2.09 (m, 1H), 1.75/1.65 (m+m, 2H), 1.70/1.28 (m+m, 2H), 1.51/1.45 (m+m, 2H), 1.10 (d, 3H), 1.08 (d, 3H). HRMS calculated for C₃₅H₃₉N₂O₃FCl₂: 624.2322; found: 625.2388 (M+H).

Using General procedure 32 and Example 748M as the appropriate indene and Preparation 2a1 as the appropriate alcohol, Example 749 was obtained. ¹H NMR (500 MHz, DMSO-d₆) δ ppm: 12.77 (br s, 1H), 8.14 (d, 1H), 7.38 (t, 1H), 7.18 (d, 1H), 7.04 (t, 1H), 6.76 (d, 1H), 6.61 (t, 1H), 6.54 (m, 2H), 6.27 (br s, 1H), 3.87 (m, 2H), 3.10/2.58 (m+m, 2H), 3.02 (m, 1H), 2.75/2.65 (m+m, 2H), 2.43-1.39 (m, 12H), 2.27 (m, 1H), 2.00 (m, 1H), 1.47/1.32 (m+m, 2H), 1.04 (d, 3H), 0.98 (d, 3H). HRMS calculated for C₃₅H₃₉N₂O₃FCl₂: 624.2322; found: 625.2387 (M+H).

### Example 750

### Example 750A 2-bromo-6,7-dichloro-2,3-dihydro-1H-inden-1-one

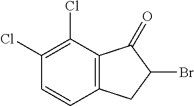 [SMILES: O=C1c2c(ccc(Cl)c2Cl)CC1Br]

Using General procedure 5 and 6,7-dichloro-2,3-dihydro-1H-inden-1-one as the appropriate indan-1-one, Example 750A was obtained. ¹H NMR (500 MHz, DMSO-d₆) δ ppm: 7.98 (d, 1H), 7.58 (dm, 1H), 5.07 (dd, 1H), 3.83/3.29 (dd+dd, 2H). HRMS calculated for C₉H₅N₂OCl₂Br: 277.8901; found: 277.8896 (M).

### Example 750B 2-bromo-6,7-dichloro-2,3-dihydro-1H-inden-1-ol

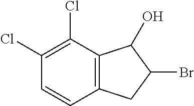 [SMILES: OC1c2c(ccc(Cl)c2Cl)CC1Br]

Using General procedure 6 and Example 745A as the appropriate bromo-indan-1-one, Example 750B was obtained as a single diastereoisomer. ¹H NMR (500 MHz, DMSO-d₆) δ ppm: 7.54 (d, 1H), 7.25 (d, 1H), 5.88 (d, 1H), 4.96 (dd, 1H), 4.74-4.67 (m, 1H), 3.37 (dd, 1H), 3.24 (dd, 1H).

### Example 750C 2-bromo-4,5-dichloro-1H-indene

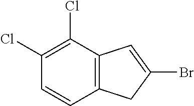 [SMILES: Clc1ccc2c(c1Cl)C=C(Br)C2]

Using General procedure 7 and Example 750B as the appropriate indane, Example 750C was obtained.

### Example 750D 2″-bromo-4″,5″-dichlorodispiro[[1,3]dioxolane-2,1′-cyclohexane-4′,1″-indene] and 2″-bromo-6″,7″-dichlorodispiro[[1,3]dioxolane-2,1′-cyclohexane-4′,1″-indene]

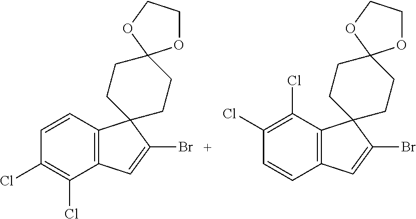 [SMILES: Clc1ccc2c(c1Cl)C=C(Br)C21CCC2(CC1)OCCO2 ; Clc1ccc2c(c1Cl)C1(CCC3(CC1)OCCO3)C(Br)=C2]

Using General procedure 8a and Example 745C as the appropriate indene, Example 750D was obtained as a mixture of regioisomers. ¹H NMR (500 MHz, DMSO-d₆) δ ppm: 7.55 (d, 1H), 7.29 (d, 1H), 7.07 (s, 1H), 3.90 (s, 4H), 3.05 (td, 2H), 2.54 (td, 2H), 1.72 (dm, 2H), 1.41 (dm, 2H).

### Example 750E 2′-bromo-4′,5′-dichlorospiro[cyclohexane-1,1′-inden]-4-one

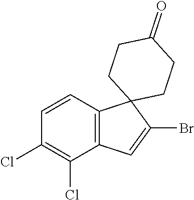 [SMILES: O=C1CCC2(CC1)C(Br)=Cc1c2ccc(Cl)c1Cl]

Using General procedure 9 and Example 750D as the appropriate ketal, a mixture of regioisomers was obtained. The regioisomers were separated via flash chromatography using hexane and EtOAc as eluents. The regioisomer eluting later was collected as Example 750E. ¹H NMR (500 MHz, DMSO-d₆) δ ppm: 7.93 (d, 1H), 7.47 (d 1H), 7.20 (s, 1H), 2.86 (ddd, 2H), 2.57-2.48 (m, 2H), 2.22 (td, 2H), 1.67 (dm, 2H).

### Example 750F 2″-bromo-4″,5″-dichlorodispiro[imidazolidine-4,1′-cyclohexane-4′,1″-indene]-2,5-dione

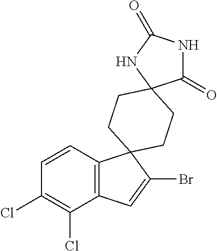 [SMILES: O=C1NC(=O)C2(CCC3(CC2)C(Br)=Cc2c3ccc(Cl)c2Cl)N1]

Using General procedure 14 and Example 750E as the appropriate ketone, Example 750F was obtained as a mixture of diastereoisomers. ¹H NMR (500 MHz, DMSO-d₆) δ ppm: 10.80/10.64 (s, 1H), 8.95/8.43 (s/s, 1H), 7.85/7.63 (d/d, 1H), 7.57/7.51 (d/d, 1H), 7.16/7.14 (s/s, 1H), 2.40-1.18 (m, 8H).

### Example 750G 4-amino-2′-bromo-4′,5′-dichlorospiro[cyclohexane-1,1′-indene]-4-carboxylic acid

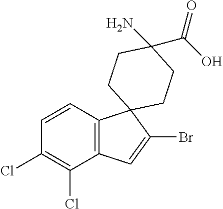 [SMILES: NC1(C(=O)O)CCC2(CC1)C(Br)=Cc1c2ccc(Cl)c1Cl]

Using General procedure 15 and Example 750F as the appropriate hydantoin, Example 750G was obtained as a mixture of diastereoisomers. ¹H NMR (500 MHz, DMSO-d₆) δ ppm: 7.98 (d, 1H), 7.51/7.44 (d/d, 1H), 7.13/7.09 (s/s, 1H), 2.60-2.43 (m, 2H), 2.22/1.79 (d, 2H), 2.08/1.91 (td, 2H), 1.14 (d, 2H).

### Example 750H 2′-bromo-4′,5′-dichloro-4-(3-chloroanilino)spiro[cyclohexane-1,1′-indene]-4-carboxylic acid

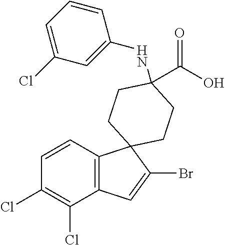 [SMILES: O=C(O)C1(Nc2cccc(Cl)c2)CCC2(CC1)C(Br)=Cc1c2ccc(Cl)c1Cl]

Using General procedure 16 and Example 750G as the appropriate amino acid and 1-chloro-3-iodo-benzene as the appropriate iodobenzene, Example 750H was obtained as a mixture of diastereoisomers. ¹H NMR (500 MHz, CDCl₃) δ ppm: 7.61-7.56 (m, 1H), 7.34-7.29 (m, 1H), 7.17/7.12 (t, 1H), 7.05-7.00 (m, 1H), 6.88/6.83 (dm/dm, 1H), 6.79/6.76 (t/t, 1H), 6.66/6.64 (dd/dd, 1H), 2.86-1.15 (m, 8H).

### Example 7501 methyl (1s,4s)-2′-bromo-4′,5′-dichloro-4-(3-chloroanilino)spiro[cyclohexane-1,1′-indene]-4-carboxylate

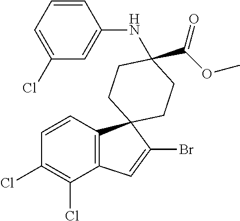 [SMILES: COC(=O)[C@]1(Nc2cccc(Cl)c2)CC[C@]2(CC1)C(Br)=Cc1c(Cl)c(Cl)ccc12]

Using General procedure 17b and Example 750H as the appropriate amino acid, a mixture of diastereoisomers was obtained. The diastereoisomers were suspended in hexane and filtered, washed with hexane. The solid material was collected as Example 7501. ¹H NMR (500 MHz, DMSO-d₆) δ ppm: 7.67 (d, 1H), 7.52 (d, 1H), 7.14 (s, 1H), 7.10 (t, 1H), 6.61 (dd, 1H), 6.60 (dd, 1H), 6.51 (s, 1H), 6.48 (dd, 1H), 3.68 (s, 3H), 2.30 (m, 4H), 2.20/1.06 (t+d, 4H). HRMS calculated for C₂₂H₁₉NO₂Cl₃Br: 512.9665; found: 513.9729 (M+H).

### Example 750J methyl (1r,4R)-4′,5′-dichloro-4-(3-chloroanilino)-2′-{(2R)-3-[(4-methoxyphenyl)methoxy]-2-methylpropyl}spiro[cyclohexane-1,1′-indene]-4-carboxylate

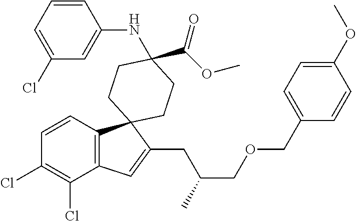 [SMILES: COc1ccc(COC[C@H](C)CC2=Cc3c(Cl)c(Cl)ccc3[C@]23CC[C@@](Nc2cccc(Cl)c2)(C(=O)OC)CC3)cc1]

Using General procedure 27b and Example 7501 as the appropriate 2-bromo-indene derivative and Preparation 3d as the appropriate Zn reagent, Example 750J was obtained. ¹H NMR (500 MHz, DMSO-d₆) δ ppm: 7.58 (d, 1H), 7.39 (d, 1H), 7.21 (dm, 2H), 7.09 (t, 1H), 6.85 (dm, 2H), 6.64 (t, 1H), 6.60 (dm, 1H), 6.54 (s, 1H), 6.48 (dm, 1H), 6.43 (s, 1H), 4.40/4.36 (d+d, 2H), 3.72 (s, 3H), 3.68 (s, 3H), 3.33/3.29 (dd+dd, 2H), 2.45-0.87 (m, 8H), 2.40/2.04 (dd+dd, 2H), 2.20 (m, 1H), 0.93 (d, 3H). HRMS calculated for C₃₄H₃₆NO₄Cl₃: 627.1710; found: 628.1777 (M+H).

### Example 750K methyl (1r,4R)-4′,5′-dichloro-4-(3-chloroanilino)-2′-[(2R)-3-hydroxy-2-methylpropyl]spiro[cyclohexane-1,1′-indene]-4-carboxylate

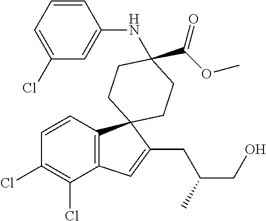 [SMILES: COC(=O)[C@]1(Nc2cccc(Cl)c2)CC[C@]2(CC1)C(C[C@@H](C)CO)=Cc1c(Cl)c(Cl)ccc12]

Using General procedure 28b and Example 750J as the appropriate PMB derivative, Example 750K was obtained. ¹H NMR (500 MHz, DMSO-d₆) δ ppm: 7.58 (d, 1H), 7.39 (d, 1H), 7.10 (t, 1H), 6.63 (t, 1H), 6.60 (dm, 1H), 6.54 (s, 1H), 6.47 (dm, 1H), 6.43 (s, 1H), 4.58 (br s, 1H), 3.68 (s, 3H), 3.33/3.30 (dd+dd, 2H), 2.40/1.95 (dd+dd, 2H), 2.37-0.87 (m, 8H), 2.00 (m, 1H), 0.90 (d, 3H). HRMS calculated for C₂₆H₂₈NO₃Cl₃: 507.1135; found: 508.1195 (M+H).

### Example 750 (1r,4R)-4′,5′-dichloro-4-(3-chloroanilino)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

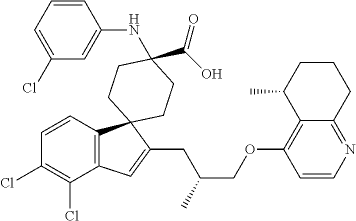 [SMILES: C[C@@H](COc1ccnc2c1[C@H](C)CCC2)CC1=Cc2c(Cl)c(Cl)ccc2[C@]12CC[C@@](Nc1cccc(Cl)c1)(C(=O)O)CC2]

Using General procedure 32 and Example 750K as the appropriate indene and Preparation 2a1 as the appropriate alcohol, Example 750 was obtained. ¹H NMR (500 MHz, DMSO-d₆) δ ppm: 12.95 (br s, 1H), 8.14 (d, 1H), 7.60 (d, 1H), 7.40 (d, 1H), 7.06 (t, 1H), 6.77 (d, 1H), 6.62 (m, 2H), 6.56 (dd, 1H), 6.55 (dd, 1H), 6.35 (br s, 1H), 4.01/3.95 (dd+dd, 2H), 3.05 (m, 1H), 2.75/2.63 (m+m, 2H), 2.53/2.25 (dd+dd, 2H), 2.48 (m, 1H), 2.34-0.86 (m, 8H), 1.78/1.70 (m+m, 2H), 1.59/1.53 (m+m, 2H), 1.11 (d, 3H), 1.10 (d, 3H). HRMS calculated for C₃₅H₃₇N₂O₃Cl₃: 638.1870; found: 639.1940 (M+H).

### Example 751 and Example 752

### Example 751A methyl (1r,4R)-4′,5′-dichloro-4-(3-chloroanilino)-2′-[(2R)-3-hydroxy-2-methylpropyl]-2′,3′-dihydrospiro[cyclohexane-1,1′-indene]-4-carboxylate, diastereoisomer 1

### Example 751B methyl (1r,4R)-4′,5′-dichloro-4-(3-chloroanilino)-2′-[(2R)-3-hydroxy-2-methylpropyl]-2′,3′-dihydrospiro[cyclohexane-1,1′-indene]-4-carboxylate, diastereoisomer 2

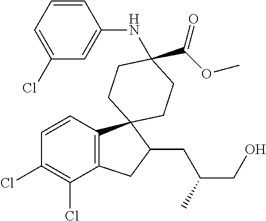

Using General procedure 19 and Example 750K as the appropriate indene, a mixture of diastereoisomers was obtained. The diastereoisomers were separated by chiral chromatography. Column: IC, 50×500 mm, 20 μm, Eluents: nPrOH/heptane. The diastereoisomer eluting earlier was collected as Example 751A. ¹H NMR (500 MHz, DMSO-d₆) δ ppm: 7.44 (d, 1H), 7.35 (d, 1H), 7.06 (t, 1H), 6.58 (t, 1H), 6.57 (dm, 1H), 6.45 (dm, 1H), 6.30 (s, 1H), 4.41 (t, 1H), 3.64 (s, 3H), 3.40/3.21 (m+m, 2H), 3.07/2.61 (dd+dd, 2H), 2.43-1.28 (m, 8H), 2.19 (m, 1H), 1.61 (m, 1H), 1.45/1.05 (m+td, 2H), 0.91 (d, 3H). HRMS calculated for C₂₆H₃₀NO₃Cl₃: 509.1291; found: 510.1353 (M+H).

The diastereoisomer eluting later was collected as Example 751B. ¹H NMR (500 MHz, DMSO-d₆) δ ppm: 7.45 (d, 1H), 7.34 (d, 1H), 7.06 (t, 1H), 6.58 (t, 1H), 6.57 (dm, 1H), 6.44 (dm, 1H), 6.32 (s, 1H), 4.47 (m, 1H), 3.64 (s, 3H), 3.24/3.2 (dd+dd, 2H), 3.04/2.57 (dd+dd, 2H), 2.44-1.32 (m, 8H), 2.17 (m, 1H), 1.56 (m, 1H), 1.37/1.07 (m+m, 2H), 0.85 (d, 3H). HRMS calculated for C₂₆H₃₀NO₃Cl₃: 509.1291; found: 510.1352 (M+H).

### Example 751 (1r,4R)-4′,5′-dichloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 752 (1r,4R)-4′,5′-dichloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

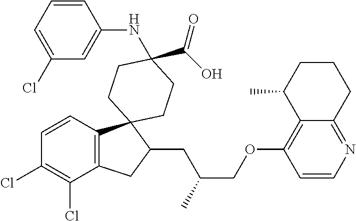

Using General procedure 32 and Example 751A as the appropriate indene and Preparation 2a1 as the appropriate alcohol, Example 751 was obtained. ¹H NMR (500 MHz, DMSO-d₆) δ ppm: 12.83 (br s, 1H), 8.13 (d, 1H), 7.45 (d, 1H), 7.36 (d, 1H), 7.02 (t, 1H), 6.77 (d, 1H), 6.60 (t, 1H), 6.52 (dm, 1H), 6.49 (dm, 1H), 6.13 (br s, 1H), 4.00/3.90 (dd+dd, 2H), 3.11/2.65 (dd+dd, 2H), 3.00 (m, 1H), 2.73/2.61 (m+m, 2H), 2.39-1.30 (m, 12H), 2.18 (m, 1H), 2.08 (m, 1H), 1.70/1.28 (m+m, 2H), 1.10 (d, 3H), 1.07 (d, 3H). HRMS calculated for C₃₅H₃₉N₂O₃Cl₃: 640.2026; found: 641.2105 (M+H).

Using General procedure 32 and Example 751B as the appropriate indene and Preparation 2a1 as the appropriate alcohol, Example 752 was obtained. ¹H NMR (500 MHz, DMSO-d₆) δ ppm: 12.77 (br s, 1H), 8.14 (d, 1H), 7.45 (d, 1H), 7.29 (d, 1H), 7.03 (t, 1H), 6.76 (d, 1H), 6.62 (t, 1H), 6.54 (m, 2H), 6.24 (br s, 1H), 3.89/3.84 (dd+dd, 2H), 3.11/2.63 (dd+dd, 2H), 3.01 (m, 1H), 2.75/2.64 (m+m, 2H), 2.46-1.42 (m, 12H), 2.29 (m, 1H), 2.00 (m, 1H), 1.48/1.31 (m+m, 2H), 1.04 (d, 3H), 0.93 (d, 3H). HRMS calculated for C₃₅H₃₉N₂O₃Cl₃: 640.2026; found: 641.2096 (M+H).

### Example 753

### Example 753A methyl (1s,4s)-2′-bromo-4-(3-chloroanilino)-4′-methylspiro[cyclohexane-1,1′-indene]-4-carboxylate

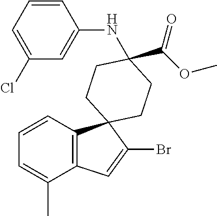 [SMILES: COC(=O)[C@]1(Nc2cccc(Cl)c2)CC[C@]2(CC1)C(Br)=Cc1c(C)cccc12]

Using General procedure 17a and Example 141 (330 mg, 0.74 mmol, 1 eq) as the appropriate amino acid, Example 753A was isolated as a white powder (290 mg, 0.63 mmol, 85%). LRMS calculated for C₂₃H₂₃BrClNO₂: 459; found: 460 (M+H). ¹H NMR (400 MHz, DMSO-d₆) δ ppm: 7.56-7.50 (m, 1H), 7.19-7.07 (m, 4H), 6.64-6.58 (m, 2H), 6.51 (s, 1H), 6.50-6.46 (m, 1H), 3.69 (s, 3H), 2.46-2.32 (m, 5H), 2.31-2.14 (m, 4H), 1.03-0.95 (m, 2H).

### Example 753B methyl (1r,4R)-4-(3-chloroanilino)-2′-{(2R)-3-[(4-methoxyphenyl)methoxy]-2-methylpropyl}-4′-methylspiro[cyclohexane-1,1′-indene]-4-carboxylate

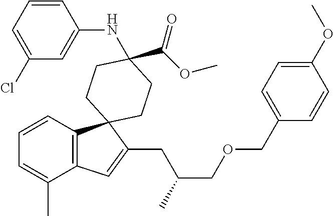 [SMILES: COc1ccc(COC[C@H](C)CC2=Cc3c(C)cccc3[C@]23CC[C@@](Nc2cccc(Cl)c2)(C(=O)OC)CC3)cc1]

Using General procedure 27a and Example 753A (290 mg, 0.63 mmol, 1 eq) as the appropriate 2-bromo-indene and Preparation 3d (1.98 mL, 0.57 M, 1.13 mmol, 1.8 eq) as the appropriate zinc reagent, Example 753B was obtained as a colourless glass (280 mg, 0.49 mmol, 78%). LRMS calculated for C₃₅H₄₀ClNO₄: 573; found: 574 (M+H). ¹H NMR (400 MHz, DMSO-d₆) δ ppm: 7.50-7.44 (m, 1H), 7.26-7.20 (m, 2H), 7.10 (t, J=8.1 Hz, 1H), 7.06-6.98 (m, 2H), 6.89-6.84 (m, 2H), 6.64 (t, J=2.1 Hz, 1H), 6.63-6.58 (m, 1H), 6.57-6.54 (m, 1H), 6.51-6.46 (m, 1H), 6.44 (s, 1H), 4.44-4.35 (m, 2H), 3.73 (s, 3H), 3.69 (s, 3H), 3.38-3.27 (m, 2H), 2.46-2.31 (m, 6H), 2.25-1.94 (m, 6H), 0.95 (d, J=6.5 Hz, 3H), 0.90-0.81 (m, 2H).

### Example 753C methyl (1r,4R)-4-(3-chloroanilino)-2′-[(2R)-3-hydroxy-2-methylpropyl]-4′-methylspiro[cyclohexane-1,1′-indene]-4-carboxylate

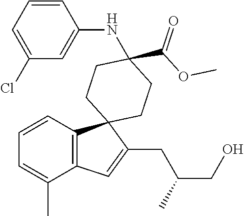 [SMILES: COC(=O)[C@]1(Nc2cccc(Cl)c2)CC[C@]2(CC1)C(C[C@@H](C)CO)=Cc1c(C)cccc12]

Using General procedure 28b and Example 753B (280 mg, 0.49 mmol, 1 eq) as the appropriate PMB derivative, Example 753C was obtained as a colourless gum (210 mg, 0.46 mmol, 95%). LRMS calculated for C₂₇H₃₂ClNO₃: 453; found: 454 (M+H). ¹H NMR (400 MHz, DMSO-d₆) δ ppm: 7.47 (d, J=6.6 Hz, 1H), 7.10 (t, J=8.1 Hz, 1H), 7.07-6.98 (m, 2H), 6.63 (t, J=2.1 Hz, 1H), 6.62-6.58 (m, 1H), 6.58-6.54 (m, 1H), 6.51-6.45 (m, 1H), 6.44 (s, 1H), 4.61-4.51 (m, 1H), 3.69 (s, 3H), 3.42-3.26 (m, 2H), 2.47-2.31 (m, 6H), 2.25-1.87 (m, 6H), 0.95-0.80 (m, 5H).

### Example 753D methyl (1r,4R)-4-(3-chloroanilino)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylate

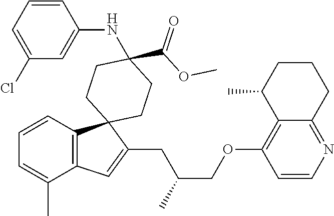 [SMILES: COC(=O)[C@]1(Nc2cccc(Cl)c2)CC[C@]2(CC1)C(C[C@@H](C)COc1ccnc3c1[C@H](C)CCC3)=Cc1c(C)cccc12]

Using General procedure 30a and Example 753C (210 mg, 0.46 mmol, 1 eq) as the appropriate indene and Preparation 2a1 (151 mg, 0.93 mmol, 2 eq) as the appropriate alcohol, Example 753D was obtained as a colourless gum (139 mg, 0.23 mmol, 50%) that was used directly in the subsequent step without further purification. LRMS calculated for C₃₇H₄₃ClN₂O₃: 598; found: 599 (M+H).

### Example 753 (1r,4R)-4-(3-chloroanilino)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

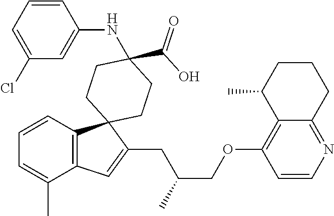 [SMILES: Cc1cccc2c1C=C(C[C@@H](C)COc1ccnc3c1[C@H](C)CCC3)[C@]21CC[C@](Nc2cccc(Cl)c2)(C(=O)O)CC1]

Using General procedure 33b and Example 753D (138 mg, 0.23 mmol, 1 eq) as the appropriate ester, Example 753 was obtained as a cream powder (34.4 mg, 0.06 mmol, 26%). LRMS calculated for C₃₆H₄₁ClN₂O₃: 584; found: 585 (M+H). ¹H NMR (400 MHz, DMSO-d₆) δ ppm: 12.84 (br s, 1H), 8.14 (d, J=5.6 Hz, 1H), 7.54-7.46 (m, 1H), 7.10-6.97 (m, 3H), 6.78 (d, J=5.6 Hz, 1H), 6.66-6.60 (m, 2H), 6.59-6.51 (m, 2H), 6.32 (br s, 1H), 4.03 (dd, J=9.6, 4.2 Hz, 1H), 3.95 (dd, J=9.4, 4.8 Hz, 1H), 3.12-3.03 (m, 1H), 2.80-2.72 (m, 1H), 2.69-2.58 (m, 1H), 2.52-2.29 (m, 7H), 2.27-2.01 (m, 5H), 1.88-1.66 (m, 2H), 1.66-1.50 (m, 2H), 1.15-1.09 (m, 6H), 0.92-0.77 (m, 2H).

### Example 754 and Example 755

### Example 754A methyl (1r,4R)-4-(3-chloroanilino)-2′-[(2R)-3-hydroxy-2-methylpropyl]-4′-methyl-2′,3′-dihydrospiro[cyclohexane-1,1′-indene]-4-carboxylate, diastereoisomer 1

and

### Example 755A methyl (1r,4R)-4-(3-chloroanilino)-2′-[(2R)-3-hydroxy-2-methylpropyl]-4′-methyl-2′,3′-dihydrospiro[cyclohexane-1,1′-indene]-4-carboxylate, diastereoisomer 2

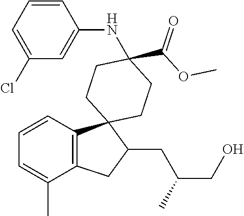

Using General procedure 19 and Example 753C (1.64 g, 3.61 mmol, 1 eq) as the appropriate indene, a mixture of diastereoisomers was obtained. They were purified and separated via prep RP-HPLC using 5 mM aq. NH₄HCO₃ solution and MeCN as eluents. The diastereoisomer eluting earlier was collected as Example 754A, white powder (278 mg, 0.61 mmol). ¹H NMR (400 MHz, DMSO-d₆) δ ppm: 7.19 (d, 1H), 7.06 (m, 2H), 6.96 (d, 1H), 6.58 (t, 1H), 6.56 (dm, 1H), 6.44 (dm, 1H), 6.31 (s, 1H), 4.46 (t, 1H), 3.64 (s, 3H), 3.25/3.21 (m+m, 2H), 2.88/2.38 (dd+dd, 2H), 2.50-1.22 (m, 8H), 2.19 (s, 3H), 2.04 (m, 1H), 1.60 (m, 1H), 1.37/1.08 (m+m, 2H), 0.86 (d, 3H). HRMS calculated for C₂₇H₃₄ClNO₃: 455.2227; found: 456.2298 (M+H).

The diastereoisomer eluting later was collected as Example 755A, white powder (294 mg, 0.64 mmol). ¹H NMR (400 MHz, DMSO-d₆) δ ppm: 7.20 (d, 1H), 7.06 (t, 1H), 7.05 (t, 1H), 6.96 (d, 1H), 6.58 (t, 1H), 6.56 (dm, 1H), 6.44 (dm, 1H), 6.29 (s, 1H), 4.38 (t, 1H), 3.64 (s, 3H), 3.43/3.20 (m+m, 2H), 2.91/2.42 (dd+dd, 2H), 2.50-1.17 (m, 8H), 2.19 (s, 3H), 2.05 (m, 1H), 1.64 (m, 1H), 1.46/1.04 (m+m, 2H), 0.92 (d, 3H). HRMS calculated for C₂₇H₃₄ClNO₃: 455.2227; found: 456.2292 (M+H).

### Example 754B methyl (1r,4R)-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate, diastereoisomer 1

and

### Example 755B methyl (1r,4R)-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate, diastereoisomer 2

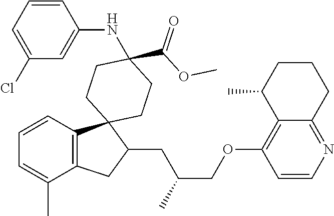

Using General procedure 30a and Example 754A (200 mg, 0.44 mmol, 1 eq) as the appropriate indane and Preparation 2a1 (143 mg, 0.88 mmol, 2 eq) as the appropriate alcohol, Example 754B was obtained as a white powder (187 mg, 0.31 mmol, 71%). LRMS calculated for C₃₇H₄₅ClN₂O₃: 600; found: 601 (M+H). ¹H NMR (400 MHz, DMSO-d₆) δ ppm: 8.15 (d, J=5.5 Hz, 1H), 7.23-7.17 (m, 1H), 7.10-7.02 (m, 2H), 7.00-6.95 (m, 1H), 6.78 (d, J=5.6 Hz, 1H), 6.60-6.53 (m, 2H), 6.47-6.42 (m, 1H), 6.35 (s, 1H), 3.95-3.85 (m, 2H), 3.65 (s, 3H), 3.10-3.00 (m, 1H), 2.95 (dd, J=15.7, 7.2 Hz, 1H), 2.82-2.72 (m, 1H), 2.72-2.59 (m, 1H), 2.51-2.38 (m, 2H), 2.23-1.95 (m, 7H), 1.95-1.42 (m, 9H), 1.41-1.29 (m, 2H), 1.06 (d, J=6.7 Hz, 3H), 1.03 (d, J=6.9 Hz, 3H).

Using General procedure 30a and Example 755A (200 mg, 0.44 mmol, 1 eq) as the appropriate indane and Preparation 2a1 (143 mg, 0.88 mmol, 2 eq) as the appropriate alcohol, Example 755B was obtained as a white powder (199 mg, 0.33 mmol, 75%). LRMS calculated for C₃₇H₄₅ClN₂O₃: 600; found: 601 (M+H). ¹H NMR (400 MHz, DMSO-d₆) δ ppm: 8.14 (d, J=5.5 Hz, 1H), 7.26-7.20 (m, 1H), 7.10-7.02 (m, 2H), 7.00-6.95 (m, 1H), 6.79 (d, J=5.6 Hz, 1H), 6.60-6.54 (m, 2H), 6.44-6.39 (m, 1H), 6.27 (s, 1H), 4.02 (dd, J=9.6, 4.2 Hz, 1H), 3.89 (dd, J=9.6, 5.3 Hz, 1H), 3.64 (s, 3H), 3.06-2.91 (m, 2H), 2.79-2.69 (m, 1H), 2.66-2.55 (m, 1H), 2.51-2.37 (m, 2H), 2.20 (s, 3H), 2.17-1.97 (m, 4H), 1.95-1.61 (m, 6H), 1.58-1.34 (m, 3H), 1.34-1.22 (m, 2H), 1.13 (d, J=6.7 Hz, 3H), 1.09 (d, J=6.9 Hz, 3H).

### Example 754 (1r,4R)-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 755 (1r,4R)-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

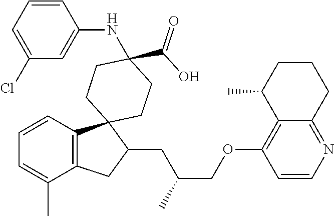

Using General procedure 33b and Example 754B (187 mg, 0.31 mmol, 1 eq) as the appropriate ester, Example 754 was obtained as a white powder (123 mg, 0.21 mmol, 67%). ¹H NMR (500 MHz, DMSO-d₆) δ ppm: 12.69 (br s, 1H), 8.14 (d, 1H), 7.18 (d, 1H), 7.06 (t, 1H), 7.03 (t, 1H), 6.97 (d, 1H), 6.77 (d, 1H), 6.61 (t, 1H), 6.53 (dm, 1H), 6.53 (dm, 1H), 6.23 (s, 1H), 3.91/3.87 (dd+dd, 2H), 3.04 (m, 1H), 2.94/2.41 (dd+dd, 2H), 2.76/2.65 (m+m, 2H), 2.5-1.31 (m, 12H), 2.18 (s, 3H), 2.13 (m, 1H), 2.03 (m, 1H), 1.49/1.34 (m+m, 2H), 1.05 (d, 3H), 1.02 (d, 3H). HRMS calculated for C₃₆H₄₃N₂O₃Cl: 586.2962; found: 587.3027 (M+H).

Using General procedure 33b and Example 755B (199 mg, 0.33 mmol, 1 eq) as the appropriate ester, Example 755 was obtained as a white powder (116 mg, 0.2 mmol, 60%). LRMS calculated for C₃₆H₄₃ClN₂O₃: 586; found: 587 (M+H). ¹H NMR (400 MHz, DMSO-d₆) δ ppm: 12.75 (br s, 1H), 8.14 (d, J=5.7 Hz, 1H), 7.31-7.17 (m, 1H), 7.11-7.01 (m, 2H), 7.01-6.93 (m, 1H), 6.79 (d, J=5.7 Hz, 1H), 6.64-6.58 (m, 1H), 6.58-6.47 (m, 2H), 6.17 (br s, 1H), 4.02 (dd, J=9.7, 4.3 Hz, 1H), 3.90 (dd, J=9.6, 5.3 Hz, 1H), 3.07-2.91 (m, 2H), 2.79-2.36 (m, 4H), 2.20 (s, 3H), 2.17-1.95 (m, 4H), 1.94-1.60 (m, 6H), 1.59-1.22 (m, 5H), 1.12 (d, J=6.7 Hz, 3H), 1.09 (d, J=6.9 Hz, 3H).

### Example 756

### Example 756A methyl (1s,4s)-2′-bromo-4-[(3-chlorophenyl)(trifluoroacetyl)amino]-4′-methylspiro[cyclohexane-1,1′-indene]-4-carboxylate

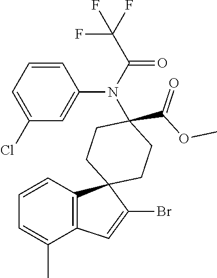 [SMILES: COC(=O)[C@]1(N(C(=O)C(F)(F)F)c2cccc(Cl)c2)CC[C@]2(CC1)C(Br)=Cc1c(C)cccc12]

To a solution of Example 753A (646 mg, 1.4 mmol, 1 eq) in DCM (15 mL) was added TEA (0.97 mL, 7.01 mmol, 5 eq) and DMAP (9 mg, 0.07 mmol, 0.05 eq), followed by TFAA (1.95 mL, 14.02 mmol, 10 eq) and the mixture was stirred at rt for 48 h. The mixture was partitioned between DCM and sat. aq. NaHCO₃ solution, and the organic phase was washed with brine, dried (MgSO₄) and concentrated in vacuo. Purification by reverse phase automated flash chromatography (CombiFlash Rf, C18 13 g RediSep column) eluting with a gradient of 10-100% MeCN in water afforded Example 756A as a yellow foam (552 mg, 0.99 mmol, 71%). ¹H NMR (400 MHz, DMSO-d₆) δ ppm: 7.84-7.79 (m, 1H), 7.73-7.60 (m, 3H), 7.33-7.27 (m, 1H), 7.18-7.11 (m, 2H), 7.10-7.05 (m, 1H), 3.84 (s, 3H), 2.54-2.23 (m, 7H), 1.79-1.66 (m, 1H), 1.60-1.35 (m, 3H).

### Example 756B methyl (1s,4s)-6′-acetyl-2′-bromo-4-[(3-chlorophenyl)(trifluoroacetyl)amino]-4′-methylspiro[cyclohexane-1,1′-indene]-4-carboxylate

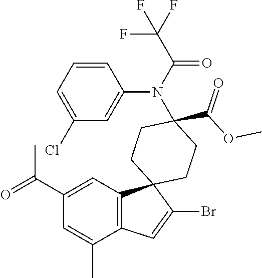 [SMILES: COC(=O)[C@]1(N(C(=O)C(F)(F)F)c2cccc(Cl)c2)CC[C@]2(CC1)C(Br)=Cc1c(C)cc(C(C)=O)cc12]

Using General procedure 23 and Example 756A (477 mg, 0.86 mmol, 1 eq) as the appropriate indene, Example 756B was obtained as a white solid (499 mg, 0.83 mmol, 97%). LRMS calculated for C₂₇H₂₄BrClF₃NO₄: 597; found: 598 (M+H). ¹H NMR (400 MHz, DMSO-d₆) δ ppm: 7.95-7.91 (m, 1H), 7.85-7.82 (m, 1H), 7.78-7.75 (m, 1H), 7.73-7.67 (m, 2H), 7.67-7.61 (m, 1H), 7.27 (s, 1H), 3.87 (s, 3H), 2.64-2.48 (m, 4H), 2.48-2.37 (m, 5H), 2.36-2.24 (m, 1H), 1.83-1.70 (m, 1H), 1.62-1.35 (m, 3H).

### Example 756C methyl (1r,4R)-6′-acetyl-4-[(3-chlorophenyl)(trifluoroacetyl)amino]-2′-[(2R)-3-hydroxy-2-methylpropyl]-4′-methylspiro[cyclohexane-1,1′-indene]-4-carboxylate

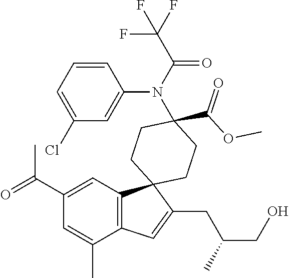 [SMILES: COC(=O)[C@]1(N(C(=O)C(F)(F)F)c2cccc(Cl)c2)CC[C@]2(CC1)C(C[C@@H](C)CO)=Cc1c(C)cc(C(C)=O)cc12]

Using General procedure 27a and Example 756B (499 mg, 0.99 mmol, 1 eq) as the appropriate 2-bromo-indene and Preparation 3d (3.13 mL, 0.57 M, 1.78 mmol, 1.8 eq) as the appropriate zinc reagent, afforded an intermediate which was treated according to General procedure 28b to afford Example 756C as a yellow gum (73.9 mg, 0.12 mmol, 18%). LRMS calculated for C₃₁H₃₃ClF₃NO₅: 591; found: 592 (M+H). ¹H NMR (400 MHz, DMSO-d₆) δ ppm: 8.03-7.98 (m, 1H), 7.95-7.91/7.91-7.87 (m, 1H), 7.80-7.68 (m, 3H), 7.63 (t, J=8.0 Hz, 1H), 6.65-6.61 (m, 1H), 4.64-4.57 (m, 1H), 3.87 (s, 3H), 3.44-3.26 (m, 2H), 2.76-2.10 (m, 11H), 2.06-1.59 (m, 3H), 1.58-1.37 (m, 1H), 1.28-0.96 (m, 2H), 0.95-0.86 (m, 3H).

### Example 756 (1r,4R)-6′-acetyl-4-(3-chloroanilino)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

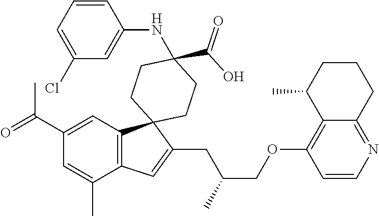 [SMILES: CC(=O)c1cc(C)c2c(c1)[C@]1(CC[C@@](Nc3cccc(Cl)c3)(C(=O)O)CC1)C(C[C@@H](C)COc1ccnc3c1[C@H](C)CCC3)=C2]

Using General procedure 30a and Example 756C (73.9 mg, 0.12 mmol, 1 eq) as the appropriate indene and Preparation 2a1 (41 mg, 0.25 mmol, 2 eq) as the appropriate alcohol, an intermediate was obtained which was hydrolyzed according to General procedure 33b to yield Example 756 as a white powder (41.6 mg, 0.07 mmol, 55%). LRMS calculated for C₃₈H₄₃ClN₂O₄: 626; found: 627 (M+H). ¹H NMR (400 MHz, DMSO-d₆) δ ppm: 12.87 (br s, 1H), 8.39 (d, J=6.3 Hz, 1H), 8.11 (s, 1H), 7.75 (s, 1H), 7.21-7.06 (m, 2H), 6.78 (s, 1H), 6.64 (t, J=2.1 Hz, 1H), 6.62-6.52 (m, 2H), 6.40 (br s, 1H), 4.26-4.09 (m, 2H), 3.14-3.04 (m, 1H), 2.92-2.82 (m, 1H), 2.81-2.70 (m, 1H), 2.64-2.35 (m, 10H), 2.35-2.15 (m, 4H), 2.15-2.01 (m, 1H), 1.89-1.70 (m, 2H), 1.66-1.52 (m, 2H), 1.18-1.10 (m, 6H), 1.00-0.83 (m, 2H).

### Example 757 and Example 758

### Example 757A methyl (1r,4r)-4-[N-(3-chlorophenyl)-2,2,2-trifluoroacetamido]-2′-[(2R)-3-hydroxy-2-methylpropyl]-4′-methyl-6′-(2-methyl-1,3-dioxolan-2-yl)spiro[cyclohexane-1,1′-indene]-4-carboxylate

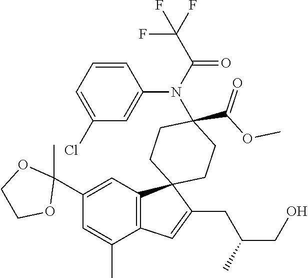 [SMILES: COC(=O)[C@]1(N(C(=O)C(F)(F)F)c2cccc(Cl)c2)CC[C@]2(CC1)C(C[C@@H](C)CO)=Cc1c(C)cc(C3(C)OCCO3)cc12]

To a solution of Example 756C (500 mg, 0.84 mmol, 1 eq) in toluene (13 mL) was added ethylene glycol (0.47 mL, 8.45 mmol, 10 eq) and PPTS (17 mg, 0.07 mmol, 0.08 eq) followed by 4A molecular sieves and the reaction was refluxed for 5 h. The reaction was allowed to cool to rt and partitioned between DCM and water. The organic phase was dried (MgSO₄), filtered and concentrated in vacuo. Purification by automated flash chromatography (Combiflash Rf, Silica 24 g RediSep™ cartridge) eluting with a gradient of 0-60% EtOAc in hexane afforded Example 757A as a colourless glass (433 mg, 0.68 mmol, 81%). LRMS calculated for C₃₃H₃₇NO₆F₃Cl: 635; found: 636 (M+H). ¹H NMR (400 MHz, DMSO-d₆) δ ppm: 7.92-7.84 (m, 1H), 7.79-7.67 (m, 2H), 7.66-7.59 (m, 1H), 7.53-7.47 (m, 1H), 7.08 (s, 1H), 6.54-6.47 (m, 1H), 4.61-4.54 (m, 1H), 4.03-3.95 (m, 2H), 3.85 (s, 3H), 3.79-3.67 (m, 2H), 3.45-3.23 (m, 2H), 2.71-2.07 (m, 8H), 2.03-1.36 (m, 7H), 1.26-0.97 (m, 2H), 0.94-0.85 (m, 3H).

### Example 757B methyl (1r,4R)-6′-acetyl-4-[(3-chlorophenyl)(trifluoroacetyl)amino]-2′-[(2R)-3-hydroxy-2-methylpropyl]-4′-methyl-2′,3′-dihydrospiro[cyclohexane-1,1′-indene]-4-carboxylate

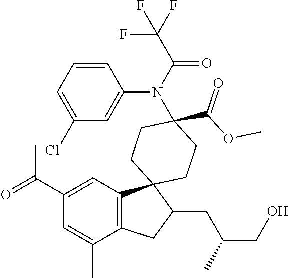

and

### Example 757C methyl (1r,4R)-4-[(3-chlorophenyl)(trifluoroacetyl)amino]-6′-(1-hydroxyethyl)-2′-[(2R)-3-hydroxy-2-methylpropyl]-4′-methyl-2′,3′-dihydrospiro[cyclohexane-1,1′-indene]-4-carboxylate

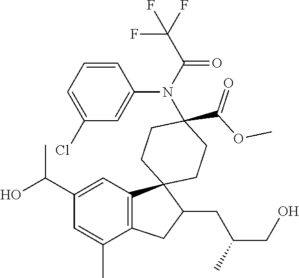

Using General procedure 19 and Example 757A as the appropriate indene, partial reduction of the ketone was observed. Example 757B and Example 757C were obtained as mixture of diastereoisomers.

### Example 757D methyl (1r,4R)-6′-acetyl-4-[(3-chlorophenyl)(trifluoroacetyl)amino]-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

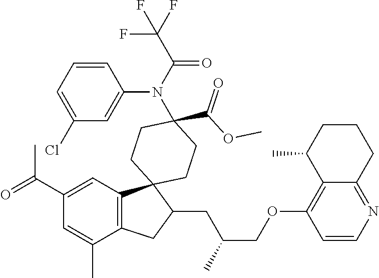

Using General procedure 30a and Example 757B as the appropriate indane and Preparation 2a1 as the appropriate alcohol, a mixture of diastereoisomers, Example 757D was isolated as a white foam (145 mg, 0.2 mmol, 29%). LRMS calculated for C₄₁H₄₆N₂O₅F₃CL: 738; found: 739 (M+H). ¹H NMR (400 MHz, DMSO-d₆) δ ppm: 8.30-8.09 (m, 1H), 7.83-7.05 (m, 6H), 6.94-6.68 (m, 1H), 4.15-3.64 (m, 5H), 3.09-1.36 (m, 24H), 1.34-0.71 (m, 9H).

### Example 757E methyl (1r,4R)-6′-acetyl-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate, diastereoisomer 1

and

### Example 758A methyl (1r,4R)-6′-acetyl-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate, diastereoisomer 2

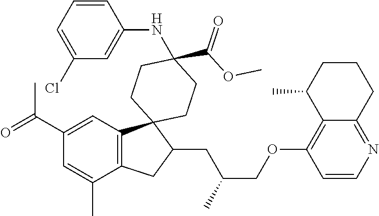

Using General procedure 33a and Example 757D as the appropriate ester, an intermediate was obtained, which was treated as described in General procedure 17a to yield a mixture of diastereoisomers. They were separated by chiral chromatography. Column: ID, 100×500 mm, 20 μm. Eluents: 10:90 DCM/EtOH+0.05% DEA. The diastereoisomer eluting earlier was collected as Example 757E. ¹H NMR (500 MHz, DMSO-d₆) δ ppm: 8.15 (d, 1H), 7.74 (s, 1H), 7.66 (s, 1H), 7.05 (t, 1H), 6.78 (d, 1H), 6.59 (t, 1H), 6.56 (dm, 1H), 6.44 (dm, 1H), 6.36 (s, 1H), 3.91/3.87 (dd+dd, 2H), 3.66 (s, 3H), 3.03 (m, 1H), 3.02/2.49 (dd+dd, 2H), 2.75/2.65 (m+m, 2H), 2.55 (s, 3H), 2.54-1.18 (m, 15H), 2.26 (s, 3H), 2.21 (m, 1H), 1.05 (d, 3H), 1.00 (d, 3H). HRMS calculated for C₃₉H₄₇N₂O₄Cl: 642.3224; found: 643.3279 (M+H).

The diastereoisomer eluting later was collected as Example 758A. HRMS calculated for C₃₉H₄₇N₂O₄Cl: 642.3224; found: 643.3287 (M+H).

### Example 757 (1r,4R)-6′-acetyl-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

### Example 758 (1r,4R)-6′-acetyl-4-(3-chloroanilino)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

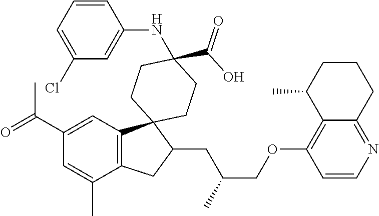

Using General procedure 33a and Example 757E as the appropriate ester, Example 757 was obtained. ¹H NMR (500 MHz, DMSO-d₆) δ ppm: 8.14 (d, 1H), 7.73 (s, 1H), 7.65 (s, 1H), 7.03 (t, 1H), 6.77 (d, 1H), 6.63 (t, 1H), 6.55 (d, 1H), 6.53 (d, 1H), 6.25 (br, 1H), 3.88 (m, 2H), 3.02 (m, 1H), 3.01/2.49 (dd+dd, 2H), 2.76/2.64 (dm+m, 2H), 2.54 (s, 3H), 2.52-1.4 (m, 8H), 2.26 (s, 3H), 2.22 (m, 1H), 2.04 (m, 1H), 1.85-1.62 (m, 2H), 1.65/1.58 (m+d, 2H), 1.46/1.35 (m+m, 2H), 1.06 (d, 3H), 1.00 (d, 3H). HRMS calculated for C₃₈H₄₅N₂O₄Cl: 628.3068; found: 629.3132 (M+H).

Using General procedure 33a and Example 758A as the appropriate ester, Example 758 was obtained. ¹H NMR (500 MHz, DMSO-d₆) δ ppm: 8.14 (d, 1H), 7.78 (s, 1H), 7.64 (s, 1H), 7.03 (t, 1H), 6.78 (d, 1H), 6.61 (t, 1H), 6.54 (dd, 1H), 6.50 (dd, 1H), 6.15 (br, 1H), 4.02/3.89 (dd+dd, 2H), 3.03/2.54 (dd+dd, 2H), 3.02 (m, 1H), 2.73/2.60 (dm+m, 2H), 2.53 (s, 3H), 2.48-1.31 (m, 8H), 2.27 (s, 3H), 2.15 (m, 1H), 2.11 (m, 1H), 1.76/1.65 (m+m, 2H), 1.72/1.25 (m+m, 2H), 1.52/1.47 (m+m, 2H), 1.11 (d, 3H), 1.08 (d, 3H). HRMS calculated for C₃₈H₄₅N₂O₄Cl: 628.3068; found: 629.3150 (M+H).

### Example 759 and Example 760

### Example 759A methyl (1r,4R)-6′-acetyl-4-(3-chloroanilino)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylate

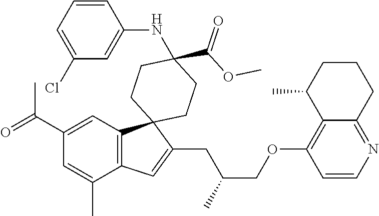 [SMILES: COC(=O)[C@]1(Nc2cccc(Cl)c2)CC[C@]2(CC1)C(C[C@@H](C)COc1ccnc3c1[C@H](C)CCC3)=Cc1c(C)cc(C(C)=O)cc12]

Using General procedure 17a and Example 756 (214 mg, 0.34 mmol, 1 eq) as the appropriate amino acid, Example 759A was obtained as a yellow gum (198.6 mg, 0.31 mmol, 91%). LRMS calculated for C₃₉H₄₅N₂O₄Cl: 640; found: 641 (M+H). ¹H NMR (400 MHz, DMSO-d₆) δ ppm: 8.15 (d, J=5.6 Hz, 1H), 8.10 (s, 1H), 7.75 (s, 1H), 7.10 (t, J=8.1 Hz, 1H), 6.79 (d, J=5.6 Hz, 1H), 6.76 (s, 1H), 6.65-6.58 (m, 2H), 6.50-6.43 (m, 2H), 4.07-3.94 (m, 2H), 3.72 (s, 3H), 3.12-3.02 (m, 1H), 2.81-2.72 (m, 1H), 2.70-2.38 (m, 11H), 2.32-2.05 (m, 5H), 1.88-1.49 (m, 4H), 1.15-1.09 (m, 6H), 0.99-0.83 (m, 2H).

### Example 759B methyl (1r,4R)-4-(3-chloroanilino)-6′-(1-hydroxyethyl)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylate, diastereoisomer 1

and

### Example 760A methyl (1r,4R)-4-(3-chloroanilino)-6′-(1-hydroxyethyl)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylate, diastereoisomer 2

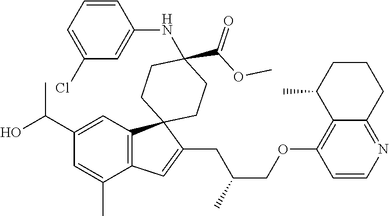 [SMILES: COC(=O)[C@]1(Nc2cccc(Cl)c2)CC[C@]2(CC1)C(C[C@@H](C)COc1ccnc3c1[C@H](C)CCC3)=Cc1c(C)cc(C(C)O)cc12]

Using General procedure 36 and Example 759A (198.6 mg, 0.31 mmol, 1 eq) as the appropriate acetyl derivative, a mixture of diastereoisomers was obtained. The diastereoisomers were separated by chiral chromatography. Column: AD, 50×500 mm, 20 μm. Eluents: 20:80 iPrOH/Heptane+0.05% DEA. The diastereoisomer eluting earlier was collected as Example 759B. ¹H NMR (500 MHz, DMSO-d₆) δ ppm: 8.13 (d, 1H), 7.49 (d, 1H), 7.08 (t, 1H), 7.01 (d, 1H), 6.77 (d, 1H), 6.62 (t, 1H), 6.59 (dm, 1H), 6.59 (s, 1H), 6.46 (dm, 1H), 6.43 (s, 1H), 5.05 (br s, 1H), 4.68 (q, 1H), 4.01/3.94 (dd+dd, 2H), 3.68 (s, 3H), 3.06 (m, 1H), 2.75/2.64 (m+m, 2H), 2.52-0.78 (m, 15H), 2.31 (s, 3H), 1.34 (d, 3H), 1.10 (d, 3H), 1.10 (d, 3H). HRMS calculated for C₃₉H₄₇N₂O₄Cl: 642.3224; found: 643.3309 (M+H).

The diastereoisomer eluting later was collected as Example 760A. ¹H NMR (500 MHz, DMSO-d₆) δ ppm: 8.13 (d, 1H), 7.50 (d, 1H), 7.08 (t, 1H), 7.01 (d, 1H), 6.76 (d, 1H), 6.62 (t, 1H), 6.59 (dm, 1H), 6.59 (s, 1H), 6.47 (dm, 1H), 6.43 (s, 1H), 5.06 (br s, 1H), 4.68 (q, 1H), 4.02/3.94 (dd+dd, 2H), 3.69 (s, 3H), 3.06 (m, 1H), 2.76/2.63 (m+m, 2H), 2.52-0.78 (m, 15H), 2.31 (s, 3H), 1.34 (d, 3H), 1.11 (d, 3H), 1.11 (d, 3H). HRMS calculated for C₃₉H₄₇N₂O₄Cl: 642.3224; found: 643.3296 (M+H).

### Example 759 (1r,4R)-4-(3-chloroanilino)-6′-(1-hydroxyethyl)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid, diastereoisomer 1

and

### Example 760 (1r,4R)-4-(3-chloroanilino)-6′-(1-hydroxyethyl)-4′-methyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid, diastereoisomer 2

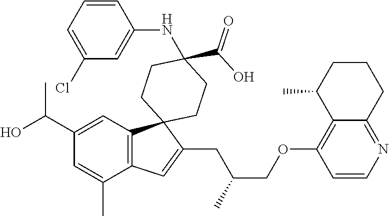 [SMILES: Cc1cc(C(C)O)cc2c1C=C(C[C@@H](C)COc1ccnc3c1[C@H](C)CCC3)[C@]21CC[C@](Nc2cccc(Cl)c2)(C(=O)O)CC1]

Using General procedure 33a and Example 759B as the appropriate ester, Example 759 was obtained. ¹H NMR (500 MHz, DMSO-d₆) δ ppm: 12.7 (br s, 1H), 8.13 (d, 1H), 7.50 (s, 1H), 7.07 (t, 1H), 7.00 (s, 1H), 6.77 (d, 1H), 6.63 (t, 1H), 6.59 (s, 1H), 6.57 (dm, 1H), 6.56 (dm, 1H), 6.34 (br s, 1H), 5.05 (d, 1H), 4.67 (m, 1H), 4.02/3.94 (dd+dd, 2H), 3.07 (m, 1H), 2.75/2.63 (m+m, 2H), 2.50-0.73 (m, 12H), 2.47/2.19 (dd+dd, 2H), 2.45 (m, 1H), 2.31 (s, 3H), 1.33 (d, 3H), 1.12 (d, 3H), 1.11 (d, 3H). HRMS calculated for C₃₈H₄₅N₂O₄Cl: 628.3068; found: 629.3133 (M+H).

Using General procedure 33a and Example 760A as the appropriate ester, Example 760 was obtained. HRMS calculated for C₃₈H₄₅N₂O₄Cl: 628.3068; found: 629.3144 (M+H).

### Example 761 (1r,4R)-4-(3-chloroanilino)-6′-(1-hydroxyethyl)-4′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

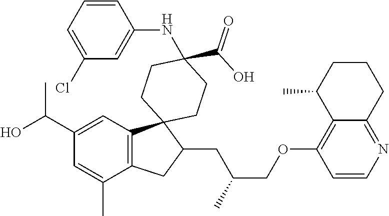

Using General procedure 30a and Example 757C as the appropriate indane and Preparation 2a1 (68.5 mg, 0.42 mmol, 2 eq) as the appropriate alcohol an intermediate was obtained which was hydrolyzed according to General procedure 33b to afford a mixture of diastereoisomers, Example 761, isolated as a white powder (7.6 mg, 0.01 mmol, 5%). LRMS calculated for C₃₈H₄₇ClN₂O₄: 630; found: 631 (M+H). ¹H NMR (400 MHz, DMSO-d₆) δ ppm: 8.09-8.04 (m, 1H), 7.40-7.25 (m, 1H), 7.11-7.01 (m, 2H), 6.80-6.66 (m, 1H), 6.65-6.51 (m, 3H), 5.64-5.49 (m, 1H), 4.48-4.35 (m, 1H), 3.46-3.14 (m, 3H), 2.92-2.28 (m, 5H), 2.22-2.15 (m, 3H), 2.15-1.17 (m, 20H), 1.12-0.99 (m, 1H), 0.95-0.81 (m, 3H).

### Example 762

### Example 762A 2′-bromo-7′-fluorospiro[cyclohexane-1,1′-inden]-4-one

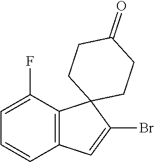 [SMILES: O=C1CCC2(CC1)C(Br)=Cc1cccc(F)c12]

Using General procedure 9 and Example 742D as the appropriate ketal, Example 762A was obtained. ¹H NMR (500 MHz, DMSO-d₆) δ ppm: 7.35 (m, 1H), 7.24 (d, 1H), 7.12 (d, 1H), 7.06 (dd, 1H), 2.67 (m, 4H), 2.21 (m, 2H), 1.96 (m, 2H).

### Example 762B 2″-bromo-7″-fluorodispiro[imidazolidine-4,1′-cyclohexane-4′,1″-indene]-2,5-dione

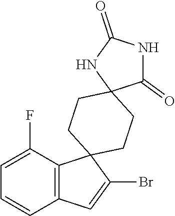 [SMILES: O=C1NC(=O)C2(CCC3(CC2)C(Br)=Cc2cccc(F)c23)N1]

Using General procedure 14 and Example 762A as the appropriate ketone, Example 762B was obtained as a mixture of diastereoisomers. ¹H NMR (500 MHz, DMSO-d₆) δ ppm: 10.57 (br s, 1H), 8.54/8.22 (s/s, 1H), 7.32 (m, 1H), 7.20/7.19 (d/d, 1H), 7.08 (m, 1H), 7.04/7.02 (dd/dd, 1H), 2.35 (m, 1H), 2.25 (m, 2H), 2.02 (m, 2H), 1.86 (m, 1H), 1.73-1.57 (m, 2H).

### Example 762C 4-amino-2′-bromo-7′-fluorospiro[cyclohexane-1,1′-indene]-4-carboxylic acid

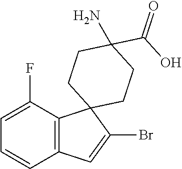 [SMILES: NC1(C(=O)O)CCC2(CC1)C(Br)=Cc1cccc(F)c12]

Using General procedure 15 and Example 762B as the appropriate hydantoin, Example 762C was obtained as a mixture of diastereoisomers. ¹H NMR (500 MHz, DMSO-d₆) δ ppm: 7.29 (m, 1H), 7.16 (d, 1H), 7.04/7.03 (d/d, 1H), 7.03-6.95 (m, 1H), 2.42 (m, 2H), 2.14 (m, 1H), 1.97-1.63 (m, 2H).

### Example 762D 2′-bromo-7′-fluoro-4-(3-chloroanilino)spiro[cyclohexane-1,1′-indene]-4-carboxylic acid

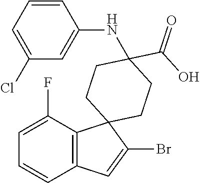 [SMILES: O=C(O)C1(Nc2cccc(Cl)c2)CCC2(CC1)C(Br)=Cc1cccc(F)c12]

Using General procedure 16 and Example 762C as the appropriate amino acid and 1-chloro-3-iodo-benzene as the appropriate iodobenzene, Example 762D was obtained as a mixture of diastereoisomers. ¹H NMR (500 MHz, DMSO-d₆) δ ppm: 12.71 (br s, 1H), 7.30 (m, 1H), 7.20/7.15 (d/d, 1H), 7.11-6.95 (m, 3H), 6.65 (m, 1H), 6.60-6.54 (m, 2H), 6.34 (br s, 1H), 2.68 (m, 1H), 2.45 (m, 1H), 2.31-2.10 (m, 4H), 1.66 (m, 1H), 1.31 (m, 1H).

### Example 762E methyl (1s,4s)-2′-bromo-7′-fluoro-4-(3-chloroanilino)spiro[cyclohexane-1,1′-indene]-4-carboxylate

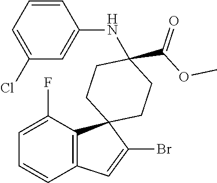 [SMILES: COC(=O)[C@]1(Nc2cccc(Cl)c2)CC[C@]2(CC1)C(Br)=Cc1cccc(F)c12]

Using General procedure 17b and Example 762D as the appropriate amino acid, a mixture of diastereoisomers was obtained. The diastereoisomers were separated by chiral chromatography. Column: (R, R) WHELK-01, 50×500 mm, 10 μm, Eluents: DCM/iPrOH/heptane. The diastereoisomer eluting later was collected as Example 762E. ¹H NMR (500 MHz, DMSO-d₆) δ ppm: 7.32 (m, 1H), 7.20 (dm, 1H), 7.08 (t, 1H), 7.06 (d, 1H), 7.04 (m, 1H), 6.61 (t, 1H), 6.59 (dm, 1H), 6.48 (dm, 1H), 6.47 (s, 1H), 3.65 (s, 3H), 2.46/2.26 (m+m, 4H), 2.19/1.29 (m+m, 4H). HRMS calculated for C₂₂H₂₀NO₂FClBr: 463.0350; found: 464.0422 (M+H).

### Example 762F methyl (1r,4R)-7′-fluoro-4-(3-chloroanilino)-2′-{(2R)-3-[(4-methoxyphenyl)methoxy]-2-methylpropyl}spiro[cyclohexane-1,1′-indene]-4-carboxylate

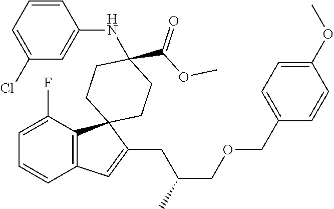 [SMILES: COc1ccc(COC[C@H](C)CC2=Cc3cccc(F)c3[C@]23CC[C@@](Nc2cccc(Cl)c2)(C(=O)OC)CC3)cc1]

Using General procedure 27b and Example 762E as the appropriate 2-bromo-indene derivative and Preparation 3d as the appropriate Zn reagent, Example 762F was obtained. ¹H NMR (500 MHz, DMSO-d₆) δ ppm: 7.23 (m, 1H), 7.21 (m, 2H), 7.12-7.05 (m, 2H), 6.89 (dd, 1H), 6.85 (m, 2H), 6.63 (t, 1H), 6.59 (dm, 1H), 6.48 (dm, 1H), 6.45 (br s, 1H), 6.39 (s, 1H), 4.39+4.36 (d+d, 2H), 3.72 (s, 3H), 3.65 (s, 3H), 3.33+3.28 (dd+dd, 2H), 2.49-2.33 (m, 3H), 2.20-1.97 (m, 6H), 1.09 (br d, 2H), 0.93 (d, 3H). HRMS calculated for C₃₄H₃₇NO₄FCl: 577.2395; found: 578.2463 (M+H).

### Example 762G methyl (1r,4R)-7′-fluoro-4-(3-chloroanilino)-2′-[(2R)-3-hydroxy-2-methylpropyl]spiro[cyclohexane-1,1′-indene]-4-carboxylate

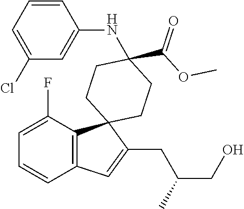 [SMILES: COC(=O)[C@]1(Nc2cccc(Cl)c2)CC[C@]2(CC1)C(C[C@@H](C)CO)=Cc1cccc(F)c12]

Using General procedure 28b and Example 762F as the appropriate PMB derivative, Example 762G was obtained. ¹H NMR (500 MHz, DMSO-d₆) δ ppm: 7.23 (m, 1H), 7.11 (dm, 1H), 7.09 (t, 1H), 6.89 (m, 1H), 6.62 (t, 1H), 6.59 (dm, 1H), 6.48 (dm, 1H), 6.47 (m, 1H), 6.39 (s, 1H), 4.56 (t, 1H), 3.65 (s, 3H), 3.34/3.29 (m+m, 2H), 2.45/2.15 (m+m, 4H), 2.39/1.94 (m+m, 2H), 2.09/1.11 (m+m, 4H), 1.95 (m, 1H), 0.89 (d, 3H). HRMS calculated for C₂₆H₂₉NO₃FCl: 457.1820; found: 458.1899 (M+H).

### Example 762H methyl (1r,2′S,4S)-4-(3-chloroanilino)-7′-fluoro-2′-[(2R)-3-hydroxy-2-methylpropyl]-2′,3′-dihydrospiro[cyclohexane-1,1′-indene]-4-carboxylate

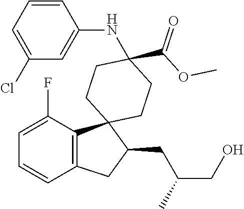 [SMILES: COC(=O)[C@]1(Nc2cccc(Cl)c2)CC[C@@]2(CC1)c1c(F)cccc1C[C@@H]2C[C@@H](C)CO]

Using General procedure 19 and Example 762G as the appropriate indane, a mixture of diastereoisomers was obtained. The diastereoisomers were separated by chiral chromatography. Column: AD, 100×500 mm, 20 μm, Eluents: EtOH/heptane. The diastereoisomer eluting later was collected as Example 762H. ¹H NMR (400 MHz, DMSO-d₆) δ ppm: 7.16 (m, 1H), 7.06 (t, 1H), 7.02 (d, 1H), 6.91 (dd, 1H), 6.60 (t, 1H), 6.56 (dm, 1H), 6.48 (dm, 1H), 6.30 (s, 1H), 4.44 (m, 1H), 3.63 (s, 3H), 3.19 (m, 2H), 3.02/2.58 (dd+dd, 2H), 2.29-1.63 (m, 8H), 2.27 (m, 1H), 1.56 (m, 1H), 1.24/1.07 (td+td, 2H), 0.88 (d, 3H). HRMS calculated for C₂₆H₃₁NO₃FCl: 459.1977; found: 460.2052 (M+H).

### Example 762 (1r,2′S,4S)-4-(3-chloroanilino)-7′-fluoro-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylic acid

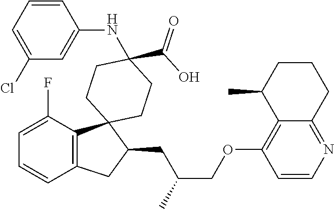 [SMILES: C[C@@H](COc1ccnc2c1[C@@H](C)CCC2)C[C@H]1Cc2cccc(F)c2[C@]12CC[C@@](Nc1cccc(Cl)c1)(C(=O)O)CC2]

Using General procedure 32 and Example 762H as the appropriate indane and Preparation 2a2 as the appropriate alcohol, Example 762 was obtained. ¹H NMR (400 MHz, DMSO-d₆) δ ppm: 8.12 (d, 1H), 7.16 (m, 1H), 7.03 (t, 1H), 7.01 (d, 1H), 6.90 (dd, 1H), 6.73 (d, 1H), 6.69 (t, 1H), 6.59 (dd, 1H), 6.53 (br d, 1H), 6.18 (br s, 1H), 3.90/3.77 (dd+dd, 2H), 3.09/2.64 (dd+dd, 2H), 2.95 (m, 1H), 2.75/2.62 (dm+m, 2H), 2.55-1.55 (m, 8H), 2.38 (m, 1H), 2.00 (m, 1H), 1.79/1.72 (m+m, 2H), 1.59 (m, 2H), 1.41-1.21 (m, 2H), 1.11 (d, 3H), 1.08 (d, 3H). HRMS calculated for C₃₅H₄₀N₂O₃FCl: 590.2712; found: 591.2774 (M+H).

### Example 763 (1r,2′S,4S)-4-(3-chloroanilino)-7′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

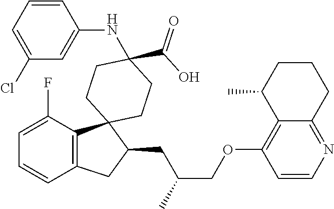 [SMILES: C[C@@H](COc1ccnc2c1[C@H](C)CCC2)C[C@H]1Cc2cccc(F)c2[C@]12CC[C@@](Nc1cccc(Cl)c1)(C(=O)O)CC2]

Using General procedure 32 and Example 762H as the appropriate indane and Preparation 2a1 as the appropriate alcohol, Example 763 was obtained. ¹H NMR (400 MHz, DMSO-d₆) δ ppm: 12.67 (br s, 1H), 8.13 (d, 1H), 7.16 (m, 1H), 7.04 (t, 1H), 7.02 (dm, 1H), 6.9 (dd, 1H), 6.75 (d, 1H), 6.66 (t, 1H), 6.58 (dm, 1H), 6.54 (dm, 1H), 6.23 (br s, 1H), 3.85 (d, 2H), 3.08/2.62 (dd+dd, 2H), 2.98 (m, 1H), 2.74/2.64 (m+m, 2H), 2.56-1.49 (m, 12H), 2.36 (m, 1H), 2.00 (m, 1H), 1.38/1.32 (m+m, 2H), 1.07 (d, 3H), 0.95 (d, 3H). HRMS calculated for C₃₅H₄₀N₂O₃FCl: 590.2712; found: 591.2765 (M+H).

### Example 765 (1r,2′S,4S)-4-(3-chloroanilino)-6′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

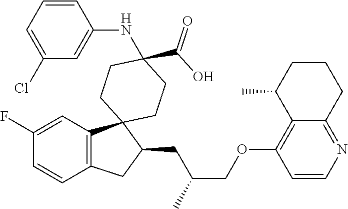 [SMILES: C[C@@H](COc1ccnc2c1[C@H](C)CCC2)C[C@H]1Cc2ccc(F)cc2[C@]12CC[C@@](Nc1cccc(Cl)c1)(C(=O)O)CC2]

Using General procedure 32 and Example 735D as the appropriate indane and Preparation 2a1 as the appropriate alcohol, Example 765 was obtained. ¹H NMR (500 MHz, DMSO-d₆) δ ppm: 12.74 (br s, 1H), 8.14 (d, 1H), 7.22 (dd, 1H), 7.09 (dd, 1H), 7.04 (t, 1H), 6.97 (m, 1H), 6.76 (d, 1H), 6.61 (t, 1H), 6.54 (dm, 1H), 6.53 (dm, 1H), 6.25 (br s, 1H), 3.90/3.85 (dd+dd, 2H), 3.04 (m, 1H), 2.98/2.50 (dd+dd, 2H), 2.82-1.32 (m, 14H), 2.19 (m, 1H), 1.98 (m, 1H), 1.48/1.33 (m+m, 2H), 1.04 (d, 3H), 1.04 (d, 3H). HRMS calculated for C₃₅H₄₀N₂O₃FCl: 590.2712; found: 591.2791 (M+H).

### Example 766 (1r,2′R,4R)-4-(3-chloroanilino)-6′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

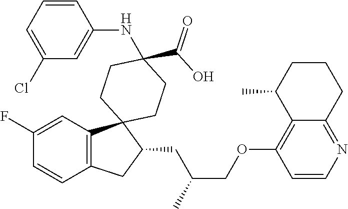 [SMILES: C[C@@H](COc1ccnc2c1[C@H](C)CCC2)C[C@@H]1Cc2ccc(F)cc2[C@]12CC[C@@](Nc1cccc(Cl)c1)(C(=O)O)CC2]

Using General procedure 32 and Example 735E as the appropriate indane and Preparation 2a1 as the appropriate alcohol, Example 766 was obtained. ¹H NMR (500 MHz, DMSO-d₆) δ ppm: 12.69 (br s, 1H), 8.13 (d, 1H), 7.22 (dd, 1H), 7.12 (dd, 1H), 7.04 (t, 1H), 6.96 (m, 1H), 6.78 (d, 1H), 6.60 (t, 1H), 6.54 (dm, 1H), 6.49 (dm, 1H), 6.16 (br s, 1H), 4.00/3.87 (dd+dd, 2H), 3.00 (m, 1H), 2.98/2.56 (dd+dd, 2H), 2.78-1.17 (m, 17H), 2.13 (m, 1H), 1.10 (d, 3H), 1.08 (d, 3H). HRMS calculated for C₃₅H₄₀N₂O₃FCl: 590.2712; found: 591.2777 (M+H).

### Example 767

### Example 767A methyl (1r,4R)-2′-[(2R)-3-{[tert-butyl(diphenyl)silyl]oxy}-2-methylpropyl]-4-(3-chloroanilino)-5′-fluorospiro[cyclohexane-1,1′-indene]-4-carboxylate

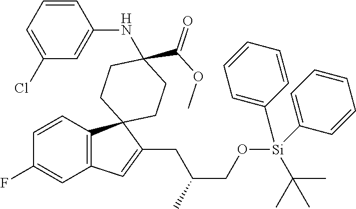 [SMILES: COC(=O)[C@]1(Nc2cccc(Cl)c2)CC[C@]2(CC1)C(C[C@@H](C)CO[Si](c1ccccc1)(c1ccccc1)C(C)(C)C)=Cc1cc(F)ccc12]

Using General procedure 27b and Example 737A as the appropriate 2-bromo-indene derivative and Preparation 3b as the appropriate Zn reagent, Example 767A was obtained. ¹H NMR (500 MHz, DMSO-d₆) δ ppm: 7.62-7.34 (m, 10H), 7.60 (dd, 1H), 7.06 (m, 2H), 6.90 (m, 1H), 6.64 (t, 1H), 6.58 (dm, 1H), 6.45 (dm, 1H), 6.42 (s, 1H), 6.34 (t, 1H), 3.68 (s, 3H), 3.58/3.54 (dd+dd, 2H), 2.43-0.73 (m, 11H), 0.98 (d, 3H), 0.96 (s, 9H).

### Example 767B methyl (1r,4R)-4-(3-chloroanilino)-5′-fluoro-2′-[(2R)-3-hydroxy-2-methylpropyl]spiro[cyclohexane-1,1′-indene]-4-carboxylate

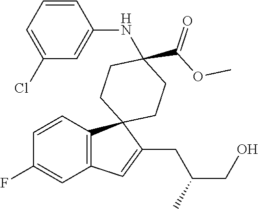 [SMILES: COC(=O)[C@]1(Nc2cccc(Cl)c2)CC[C@]2(CC1)C(C[C@@H](C)CO)=Cc1cc(F)ccc12]

Using General procedure 29 and Example 767A as the appropriate silyl derivative, Example 767B was obtained. ¹H NMR (500 MHz, DMSO-d₆) δ ppm: 7.61 (dd, 1H), 7.09 (t, 1H), 6.91 (m, 1H), 6.62 (t, 1H), 6.59 (dm, 1H), 6.47 (dm, 1H), 6.46 (m, 1H), 6.42 (s, 1H), 4.56 (br s, 1H), 3.68 (s, 3H), 3.31 (m, 2H), 2.43-0.78 (m, 11H), 0.89 (d, 3H). HRMS calculated for C₂₆H₂₉NO₃FCl: 457.1820; found: 458.1895 (M+H).

### Example 767 (1r,4R)-4-(3-chloroanilino)-5′-fluoro-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

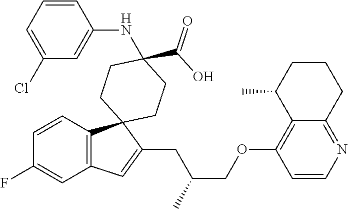 [SMILES: C[C@@H](COc1ccnc2c1[C@H](C)CCC2)CC1=Cc2cc(F)ccc2[C@]12CC[C@@](Nc1cccc(Cl)c1)(C(=O)O)CC2]

Using General procedure 32 and Example 767B as the appropriate indene and Preparation 2a1 as the appropriate alcohol, Example 767 was obtained. ¹H NMR (500 MHz, DMSO-d₆) δ ppm: 12.94 (br s, 1H), 8.13 (d, 1H), 7.64 (dd, 1H), 7.11 (dd, 1H), 7.05 (t, 1H), 6.92 (m, 1H), 6.77 (d, 1H), 6.62 (t, 1H), 6.54 (m, 3H), 6.32 (br s, 1H), 4.02/3.93 (dd+dd, 2H), 3.06 (m, 1H), 2.75/2.63 (m+m, 2H), 2.47/2.21 (dd+dd, 2H), 2.43 (m, 1H), 2.42-0.74 (m, 12H), 1.11 (d, 3H), 1.10 (d, 3H). HRMS calculated for C₃₅H₃₈N₂O₃FCl: 588.2555; found: 589.2641 (M+H).

### Example 768 (1r,2′S,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

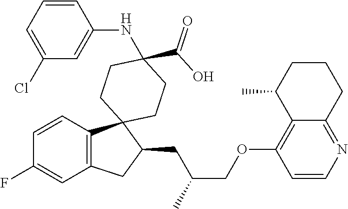 [SMILES: C[C@@H](COc1ccnc2c1[C@H](C)CCC2)C[C@H]1Cc2cc(F)ccc2[C@]12CC[C@@](Nc1cccc(Cl)c1)(C(=O)O)CC2]

Using General procedure 32 and Example 737D as the appropriate indane and Preparation 2a1 as the appropriate alcohol, Example 768 was obtained. ¹H NMR (500 MHz, DMSO-d₆) δ ppm: 12.68 (br s, 1H), 8.14 (d, 1H), 7.35 (dd, 1H), 7.04 (t, 1H), 7.03 (dd, 1H), 6.97 (m, 1H), 6.77 (d, 1H), 6.61 (t, 1H), 6.53 (m, 2H), 6.23 (br s, 1H), 3.89/3.86 (dd+dd, 2H), 3.04 (m, 1H), 3.01/2.53 (dd+dd, 2H), 2.76/2.65 (m+m, 2H), 2.46-1.30 (m, 12H), 2.17 (m, 1H), 1.98 (m, 1H), 1.48/1.33 (m+m, 2H), 1.04 (d, 3H), 1.03 (d, 3H). HRMS calculated for C₃₅H₄₀N₂O₃FCl: 590.2712; found: 591.2774 (M+H).

### Example 769 (1r,2′R,4R)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

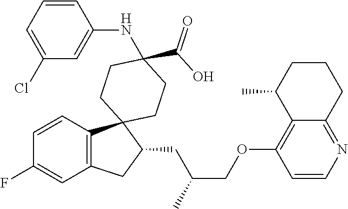 [SMILES: C[C@@H](COc1ccnc2c1[C@H](C)CCC2)C[C@@H]1Cc2cc(F)ccc2[C@]12CC[C@@](Nc1cccc(Cl)c1)(C(=O)O)CC2]

Using General procedure 32 and Example 737E as the appropriate indane and Preparation 2a1 as the appropriate alcohol, Example 769 was obtained. ¹H NMR (500 MHz, DMSO-d₆) δ ppm: 12.73 (br s, 1H), 8.13 (d, 1H), 7.38 (dd, 1H), 7.03 (m, 2H), 6.97 (m, 1H), 6.78 (d, 1H), 6.60 (t, 1H), 6.53 (dm, 1H), 6.50 (dm, 1H), 6.13 (br s, 1H), 4.00/3.87 (dd+dd, 2H), 3.01 (m, 1H), 3.01/2.59 (dd+dd, 2H), 2.73/2.60 (m+m, 2H), 2.46-1.22 (m, 12H), 2.11 (m, 1H), 2.03 (m, 1H), 1.70/1.25 (m+m, 2H), 1.10 (d, 3H), 1.09 (d, 3H). HRMS calculated for C₃₅H₄₀N₂O₃FCl: 590.2712; found: 591.2780 (M+H).

### Example 770

### Example 770A (5R,8R)-8-{[tert-butyl(dimethyl)silyl]oxy}-5-methyl-5,6,7,8-tetrahydroquinolin-4-ol

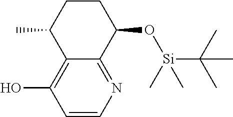 [SMILES: C[C@@H]1CC[C@@H](O[Si](C)(C)C(C)(C)C)c2nccc(O)c21]

To a solution of Example 771C (490 mg, 2.73 mmol, 1 eq) in DMF (10 mL), cooled to 0° C., was added DIPEA (1.36 mL, 8.2 mmol, 3 eq) and DMAP (33 mg, 0.27 mmol, 0.1 eq), followed by the dropwise addition of TBDMS-OTf (1.13 mL, 4.92 mmol, 1.8 eq), and the mixture was stirred at rt for 5 h. Further TBDMS-OTf (0.5 mL, 2.18 mmol, 0.8 eq) was added and then the mixture was stirred for 18 h. The mixture was partitioned between DCM and sat. aq. NaHCO₃ solution. The layers were separated, and the organic layer was dried (PTFE phase separator) and concentrated under reduced pressure. The crude product was purified by automated flash chromatography (Combiflash Rf, Silica 24 g RediSep cartridge) eluting with a gradient of 0-14% MeOH in DCM to obtain Example 770A as a cream wax (471 mg, 1.6 mmol, 59%). LRMS calculated for C₁₆H₂₆ClNOSi: 293; found: 294 (M+H). ¹H NMR (400 MHz, DMSO-d₆) δ ppm: 10.80-10.67 (m, 1H), 7.60-7.51 (m, 1H), 5.98 (d, J=7.1 Hz, 1H), 4.56-4.51 (m, 1H), 2.89-2.78 (m, 1H), 2.05-1.86 (m, 2H), 1.72-1.61 (m, 1H), 1.43-1.33 (m, 1H), 1.04 (d, J=6.8 Hz, 3H), 0.85 (s, 9H), 0.16 (s, 3H), 0.14 (s, 3H).

### Example 770B methyl (1r,4R)-2′-[(2R)-3-{[(5R,8R)-8-{[tert-butyl(dimethyl)silyl]oxy}-5-methyl-5,6,7,8-tetrahydroquinolin-4-yl]oxy}-2-methylpropyl]-4-(3-chloroanilino)-5′-fluorospiro[cyclohexane-1,1′-indene]-4-carboxylate

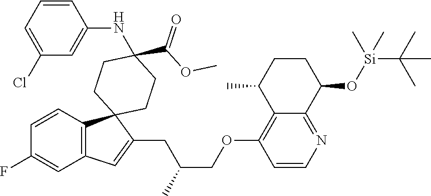 [SMILES: COC(=O)[C@]1(Nc2cccc(Cl)c2)CC[C@]2(CC1)C(C[C@@H](C)COc1ccnc3c1[C@H](C)CC[C@H]3O[Si](C)(C)C(C)(C)C)=Cc1cc(F)ccc12]

Using General procedure 30a and Example 767B as the appropriate indene and Example 770A as the appropriate alcohol, Example 770B was obtained. ¹H NMR (500 MHz, DMSO-d₆) δ ppm: 8.24 (d, 1H), 7.60 (m, 1H), 7.12 (m, 1H), 7.06 (t, 1H), 6.91 (m, 1H), 6.85 (d, 1H), 6.62 (t, 1H), 6.58 (dd, 1H), 6.55 (s, 1H), 6.45 (dd, 1H), 6.44 (s, 1H), 4.03/3.97 (dd+dd, 2H), 3.68 (s, 3H), 3.04 (m, 1H), 2.48-0.89 (m, 15H), 1.10 (d, 3H), 1.08 (d, 3H), 0.81 (s, 9H), 0.15/−0.08 (s+s, 6H).

### Example 770C methyl (1r,4R)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]spiro[cyclohexane-1,1′-indene]-4-carboxylate

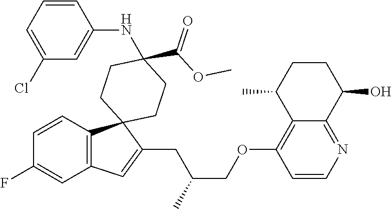 [SMILES: COC(=O)[C@]1(Nc2cccc(Cl)c2)CC[C@]2(CC1)C(C[C@@H](C)COc1ccnc3c1[C@H](C)CC[C@H]3O)=Cc1cc(F)ccc12]

Using General procedure 29 and Example 770B as the appropriate silyl derivative, Example 770C was obtained. ¹H NMR (500 MHz, DMSO-d₆) δ ppm: 8.25 (d, 1H), 7.62 (dd, 1H), 7.13 (dd, 1H), 7.08 (t, 1H), 6.92 (dd, 1H), 6.86 (d, 1H), 6.62 (t, 1H), 6.59 (dm, 1H), 6.56 (t, 1H), 6.47 (dm, 1H), 6.45 (s, 1H), 4.93 (d, 1H), 4.44 (m, 1H), 4.05/3.95 (dd+dd, 2H), 3.68 (s, 3H), 3.03 (m, 1H), 2.55-0.78 (m, 15H), 1.10 (d, 3H), 1.09 (d, 3H). HRMS calculated for C₃₆H₄₀N₂04FCl: 618.2661; found: 619.2718 (M+H).

### Example 770 (1r,4R)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]spiro[cyclohexane-1,1′-indene]-4-carboxylic acid

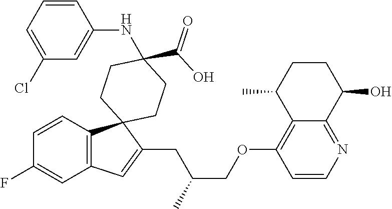 [SMILES: C[C@@H](COc1ccnc2c1[C@H](C)CC[C@H]2O)CC1=Cc2cc(F)ccc2[C@]12CC[C@@](Nc1cccc(Cl)c1)(C(=O)O)CC2]

Using General procedure 33a and Example 770C as the appropriate ester, Example 770 was obtained. ¹H NMR (500 MHz, DMSO-d₆) δ ppm: 8.24 (d, 1H), 7.70 (dd, 1H), 7.09 (dd, 1H), 6.98 (t, 1H), 6.89 (m, 1H), 6.86 (d, 1H), 6.62 (t, 1H), 6.57 (dm, 1H), 6.52 (t, 1H), 6.44 (dm, 1H), 6.08 (br s, 1H), 4.93 (br s, 1H), 4.44 (m, 1H), 4.05/3.93 (dd+dd, 2H), 3.06 (m, 1H), 2.48/2.22 (m+m, 2H), 2.44-0.70 (m, 12H), 2.43 (m, 1H), 1.12 (d, 3H), 1.11 (d, 3H). HRMS calculated for C₃₅H₃₈N₂O₄FCl: 604.2504; found: 605.2577 (M+H).

### Example 771

### Example 771A (5R,8R)-4-chloro-5-methyl-8-{[tri(propan-2-yl)silyl]oxy}-5,6,7,8-tetrahydroquinoline

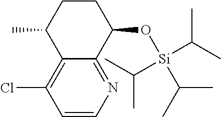 [SMILES: CC(C)[Si](O[C@@H]1CC[C@@H](C)c2c(Cl)ccnc21)(C(C)C)C(C)C]

To a solution of Example 692E (850 mg, 4.3 mmol, 1 eq) in DCM (30 mL), cooled to 0° C., was added DIPEA (2.14 mL, 12.9 mmol, 3 eq) then TIPSOTf (2.29 mL, 8.6 mmol, 2 eq) and the mixture was stirred at rt for 2 h under N₂. Sat. aq. NH₄Cl solution was added, and it was extracted with DCM. The combined organic layer was washed with brine, dried over MgSO₄, filtered and the filtrate was concentrated under reduced pressure. The crude product was purified by automated flash chromatography (Combiflash Rf, Silica 24 g RediSep cartridge) eluting with a gradient of 0-8% EtOAc in heptane to obtain Example 771A as a colourless oil (1.51 g, 4.27 mmol, 99%). LRMS calculated for C₁₉H₃₂ClNOSi: 353; found: 354 (M+H). ¹H NMR (400 MHz, DMSO-d₆) δ ppm: 8.37 (d, J=5.2 Hz, 1H), 7.44 (d, J=5.2 Hz, 1H), 4.88-4.83 (m, 1H), 3.27-3.18 (m, 1H), 2.31-2.20 (m, 1H), 2.20-2.09 (m, 1H), 1.95-1.86 (m, 1H), 1.62-1.54 (m, 1H), 1.18 (d, J=7.0 Hz, 3H), 1.16-0.94 (m, 21H).

### Example 771B (5R,8R)-5-methyl-8-{[tri(propan-2-yl)silyl]oxy}-5,6,7,8-tetrahydroquinolin-4-ol

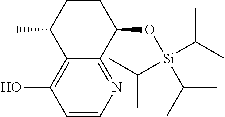 [SMILES: CC(C)[Si](O[C@@H]1CC[C@@H](C)c2c(O)ccnc21)(C(C)C)C(C)C]

and

### Example 771C (5R,8R)-5-methyl-5,6,7,8-tetrahydroquinoline-4,8-diol

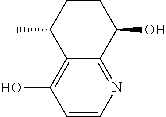

Example 771A (1.51 g, 4.27 mmol, 1 eq) and K₂CO₃ (1.77 g, 12.8 mmol, 3 eq) were combined in DMF (19 mL) and water (1.9 mL). The mixture was sparged with N₂ and then Herrmann's catalyst (80 mg, 85.31 μmol, 0.02 eq) and tBuXPhos (145 mg, 0.34 mmol, 0.08 eq) were added and the mixture was heated at 110° C. for 12 h under N₂. The mixture was allowed to cool to rt, filtered through celite, washed with 3:1 DCM/IPA (3:1). Then it was concentrated under reduced pressure and the residue was triturated with DCM. The solids were collected by filtration and dried to afford Example 771C as a white powder (300 mg, 1.67 mmol, 39%). LRMS calculated for C₁₀H₁₃NO₂: 179; found: 180 (M+H). ¹H NMR (400 MHz, DMSO-d₆) δ ppm: 7.48 (d, J=6.8 Hz, 1H), 5.92 (d, J=6.8 Hz, 1H), 4.37-4.32 (m, 1H), 2.87-2.78 (m, 1H), 1.98-1.85 (m, 2H), 1.65-1.56 (m, 1H), 1.36-1.27 (m, 1H), 1.05 (d, J=6.8 Hz, 3H).

The filtrate was concentrated in vacuo and purified by automated flash chromatography (Combiflash Rf, Silica 24 g RediSep cartridge) eluting with a gradient of 0-10% MeOH in DCM to afford Example 771B as a colourless glass (506 mg, 1.51 mmol, 35%). LRMS calculated for C₁₉H₃₃NO₂Si: 335; found: 336 (M+H). ¹H NMR (400 MHz, DMSO-d₆) δ ppm: 10.72-10.55 (br m, 1H), 7.64-7.57 (br m, 1H), 6.07-5.91 (br m, 1H), 4.72-4.56 (br m, 1H), 2.95-2.77 (br m, 1H), 2.13-1.92 (br m, 2H), 1.83-1.65 (br m, 1H), 1.49-1.34 (br m, 1H), 1.28-0.92 (m, 24H).

### Example 771D methyl (1r,2′S,4S)-4-(3-chloroanilino)-5′-fluoro-2′-[(2R)-2-methyl-3-{[(5R,8R)-5-methyl-8-{[tri(propan-2-yl)silyl]oxy}-5,6,7,8-tetrahydroquinolin-4-yl]oxy}propyl]-2′,3′-dihydrospiro[cyclohexane-1,1′-indene]-4-carboxylate

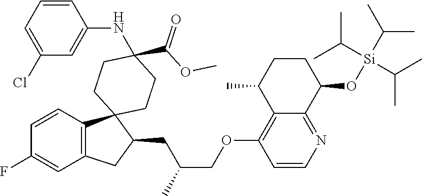 [SMILES: COC(=O)[C@]1(Nc2cccc(Cl)c2)CC[C@@]2(CC1)c1ccc(F)cc1C[C@@H]2C[C@@H](C)COc1ccnc2c1[C@H](C)CC[C@H]2O[Si](C(C)C)(C(C)C)C(C)C]

Using General procedure 30a and Example 737D as the appropriate indane and Example 771B as the appropriate alcohol, Example 771D was obtained. LRMS calculated for C₄₅H₆₂N₂O₄FClSi: 776.4; found: 777.4 (M+H).

### Example 771E methyl (1r,2′S,4S)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylate

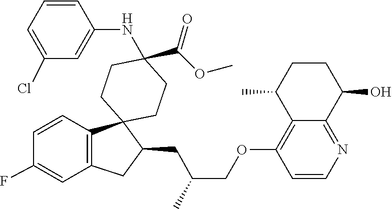 [SMILES: COC(=O)[C@]1(Nc2cccc(Cl)c2)CC[C@@]2(CC1)c1ccc(F)cc1C[C@@H]2C[C@@H](C)COc1ccnc2c1[C@H](C)CC[C@H]2O]

Using General procedure 29 and Example 771D as the appropriate silyl derivative, Example 771E was obtained. ¹H NMR (500 MHz, DMSO-d₆) δ ppm: 8.25 (d, 1H), 7.37 (dd, 1H), 7.05 (t, 1H), 7.04 (dd, 1H), 6.97 (td, 1H), 6.86 (d, 1H), 6.57 (dd, 1H), 6.56 (dd, 1H), 6.45 (dd, 1H), 6.33 (s, 1H), 4.93 (d, 1H), 4.44 (q, 1H), 3.92/3.87 (dd+dd, 2H), 3.64 (s, 3H), 3.02 (m, 1H), 3.01/2.55 (dd+dd, 2H), 2.46-1.28 (m, 8H), 2.18 (m, 1H), 2.06/1.38 (m+m, 2H), 2.00 (m, 1H), 1.94/1.72 (m+m, 2H), 1.48/1.34 (dd+dd, 2H), 1.05 (d, 3H), 1.00 (d, 3H).

### Example 771 (1r,2′S,4S)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid

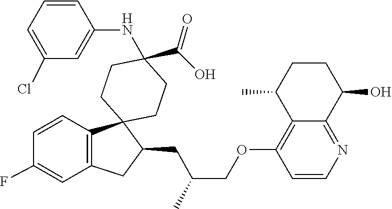 [SMILES: C[C@@H](COc1ccnc2c1[C@H](C)CC[C@H]2O)C[C@H]1Cc2cc(F)ccc2[C@]12CC[C@@](Nc1cccc(Cl)c1)(C(=O)O)CC2]

Using General procedure 33a and Example 771E as the appropriate ester, Example 771 was obtained. ¹H NMR (500 MHz, DMSO-d₆) δ ppm: 12.71 (br s, 1H), 8.25 (d, 1H), 7.35 (dd, 1H), 7.05 (dd, 1H), 7.04 (t, 1H), 6.97 (td, 1H), 6.86 (d, 1H), 6.60 (dd, 1H), 6.54 (m, 2H), 6.26 (br s, 1H), 4.93 (d, 1H), 4.44 (q, 1H), 3.92/3.86 (dd+dd, 2H), 3.02 (m, 1H), 3.01/2.55 (dd+dd, 2H), 2.44-1.34 (m, 8H), 2.18 (m, 1H), 2.06/1.38 (m+m, 2H), 2.00 (m, 1H), 1.94/1.72 (m+m, 2H), 1.48/1.34 (dd+t, 2H), 1.05 (d, 3H), 1.00 (d, 3H). HRMS calculated for C₃₅H₄₀N₂04FCl: 606.2661; found: 607.2730 (M+H).

### Example 772

### Example 772A (5R,8R)-4-chloro-8-fluoro-5-methyl-5,6,7,8-tetrahydroquinoline

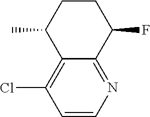

Example 772A was synthesized as described at Example 689A, using Example 692D instead of Example 673E.

### Example 772B (5R,8R)-8-fluoro-5-methyl-5,6,7,8-tetrahydroquinolin-4-ol

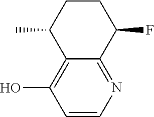

Example 772A (670 mg, 3.36 mmol, 1 eq) and K₂CO₃ (1.39 g, 10.1 mmol, 3 eq) were combined in DMF (9 mL) and water (0.9 mL). tBuXPhos (114 mg, 0.27 mmol, 0.08 eq) and Herrmann's catalyst (63 mg, 67.1 μmol, 0.02 eq) were added and the mixture was sparged with N₂ and heated at 115° C. for 18 h. The mixture was allowed to cool to rt, filtered through celite, washed with 10% DCM in MeOH, then the filtrate was concentrated in vacuo. The residue was partitioned between DCM and 2 M aq. HCl solution, and the organic phase was discarded. The aq. phase was adjusted to pH 8 with solid NaHCO₃ and saturated with solid NaCl. The mixture was extracted with DCM/iPrOH (4:1). The combined organic extracts were washed with brine, dried (MgSO₄) and concentrated in vacuo to afford Example 772B, isolated as an off-white powder (545 mg, 3.01 mmol, 90%). ¹H NMR (400 MHz, DMSO-d₆) δ ppm; 11.56 (br s, 1H), 7.80-7.61 (br m, 1H), 6.30-6.04 (br m, 1H), 5.45-5.28 (m, 1H), 3.02-2.89 (m, 1H), 2.21-1.96 (m, 2H), 1.91-1.78 (m, 1H), 1.55-1.46 (m, 1H), 1.06 (d, J=6.9 Hz, 3H). HRMS calculated for C₁₀H₁₂FNO: 181.0903; found: 182.0978 (M+H).

### Example 772 (1r,4R)-4-(3-chloroanilino)-5′-fluoro-2′-[(2R)-3-{[(5R,8R)-8-fluoro-5-methyl-5,6,7,8-tetrahydroquinolin-4-yl]oxy}-2-methylpropyl]spiro[cyclohexane-1,1′-indene]-4-carboxylic acid

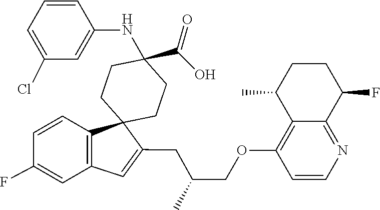 [SMILES: C[C@@H](COc1ccnc2c1[C@H](C)CC[C@H]2F)CC1=Cc2cc(F)ccc2[C@]12CC[C@@](Nc1cccc(Cl)c1)(C(=O)O)CC2]

Using General procedure 32 and Example 767B as the appropriate indene and Example 772B as the appropriate alcohol, Example 772 was obtained. ¹H NMR (500 MHz, DMSO-d₆) δ ppm: 12.85 (br s, 1H), 8.33 (d, 1H), 7.63 (dd, 1H), 7.12 (dd, 1H), 7.07 (t, 1H), 6.99 (d, 1H), 6.93 (td, 1H), 6.63 (dd, 1H), 6.57 (dd, 1H), 6.56 (s, 1H), 6.55 (dd, 1H), 6.35 (br s, 1H), 5.36 (dt, 1H), 4.08/3.99 (dd+dd, 2H), 3.13 (m, 1H), 2.50/2.23 (dd+dd, 2H), 2.45 (m, 1H), 2.40-0.81 (m, 10H), 1.89/1.46 (m+m, 2H), 1.11 (d, 3H), 1.09 (d, 3H). HRMS calculated for C₃₅H₃₇N₂O₃F₂Cl: 606.2461; found: 607.2531 (M+H).

### Example 773 (1r,2′S,4S)-4-(3-chloroanilino)-2′-[(2R)-3-{[(5R,8R)-5,8-dimethyl-5,6,7,8-tetrahydroquinolin-4-yl]oxy}-2-methylpropyl]-5′-fluoro-2′,3′-dihydrospiro[cyclohexane-1,1′-indene]-4-carboxylic acid

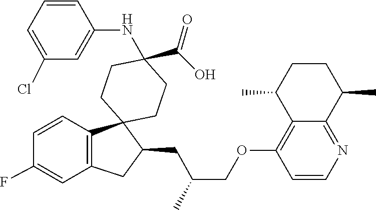 [SMILES: C[C@@H](COc1ccnc2c1[C@H](C)CC[C@H]2C)C[C@H]1Cc2cc(F)ccc2[C@]12CC[C@@](Nc1cccc(Cl)c1)(C(=O)O)CC2]

Using General procedure 32 and Example 737D as the appropriate indane and Example 658G as the appropriate alcohol, Example 773 was obtained. ¹H NMR (500 MHz, DMSO-d₆) δ ppm: 12.69 (s, 1H), 8.18 (d, 1H), 7.35 (dd, 1H), 7.04 (m, 2H), 6.97 (td, 1H), 6.75 (d, 1H), 6.61 (t, 1H), 6.53 (m, 2H), 6.25 (br s, 1H), 3.89/3.84 (dd+dd, 2H), 3.03 (m, 1H), 3.01/2.54 (dd+dd, 2H), 2.86 (m, 1H), 2.46-1.29 (m, 8H), 2.17 (m, 1H), 2.02/1.50 (tm+d, 2H), 1.99 (m, 1H), 1.86/1.43 (tm+d, 2H), 1.48-1.34 (m+m, 2H), 1.18 (d, 3H), 1.04 (d, 3H), 1.02 (d, 3H). HRMS calculated for C₃₆H₄₂N₂O₃FCl: 604.2868; found: 605.2937 (M+H).

### Example 774

### Example 774A 2-bromo-5,6-difluoro-2,3-dihydro-1H-inden-1-ol

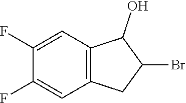 [SMILES: OC1c2cc(F)c(F)cc2CC1Br]

5,6-difluoro-1H-indene (13.77 g, 90.5 mmol) was dissolved in DMSO (140 mL) and water (14 mL), then cooled to 5° C. NBS (17.71 g, 99.55 mmol) was added portionwise over 30 min. The suspension was stirred at 5° C. for 1 h, then it was allowed to warm up to rt. Water (500 mL) was added to the suspension and the mixture was extracted with EtOAc. The combined organic layers were washed with brine, dried over Na₂SO₄, filtered and the filtrate was concentrated under reduced pressure. The product was triturated with cold MeOH, filtered and the solid material was dried to obtain Example 774A. MS (EI, 70 eV) m/z (% relative intensity, [ion]): 55 (38), 101 (23), 119 (22), 151 (55), 169 (100), 248 (21, [M+]), 250 (21, [M+]).

### Example 774B 2-bromo-5,6-difluoro-1H-indene

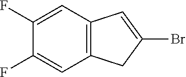 [SMILES: Fc1cc2c(cc1F)CC(Br)=C2]

Using General procedure 7 and Example 774A as the appropriate indane, Example 774B was obtained. MS (EI, 70 eV) m/z (% relative intensity, [ion]): 50 (4), 75 (22), 101 (5), 125 (5), 151 (100), 230 (15, [M+]), 252 (15, [M+]).

### Example 774C 2″-bromo-5″,6″-difluorodispiro[[1,3]dioxolane-2,1′-cyclohexane-4′,1″-indene]

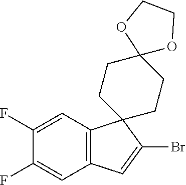 [SMILES: Fc1cc2c(cc1F)C1(CCC3(CC1)OCCO3)C(Br)=C2]

Using General procedure 8a and Example 774B as the appropriate indene, Example 774C was obtained. ¹H NMR (500 MHz, DMSO-d₆) δ ppm: 7.70 (dd, 1H), 7.43 (dd, 1H), 7.01 (s, 1H), 3.95 (s, 4H), 2.10 (td, 2H), 2.05 (td, 2H), 1.87 (br d, 2H), 1.20 (br d, 2H).

### Example 774D 2′-bromo-5′,6′-difluorospiro[cyclohexane-1,1′-inden]-4-one

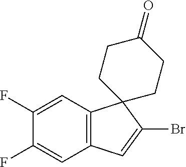 [SMILES: O=C1CCC2(CC1)C(Br)=Cc1cc(F)c(F)cc12]

Using General procedure 9 and Example 774C as the appropriate ketal, Example 774D was obtained. ¹H NMR (500 MHz, DMSO-d₆) δ ppm: 8.12 (dd, 1H), 7.46 (dd 1H), 7.07 (s, 1H), 2.91 (ddd, 2H), 2.52-2.40 (m, 2H), 2.19 (td, 2H), 1.62 (dm, 2H).

### Example 774E 2″-bromo-5″,6″-difluorodispiro[imidazolidine-4,1′-cyclohexane-4′,1″-indene]-2,5-dione

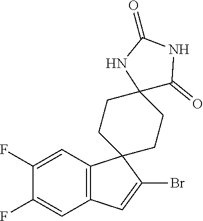 [SMILES: O=C1NC(=O)C2(CCC3(CC2)C(Br)=Cc2cc(F)c(F)cc23)N1]

Using General procedure 14 and Example 774D as the appropriate ketone, a mixture of diastereoisomers was obtained. EtOAc was added to the product and it was refluxed for 30 min, then filtered. The filtrate was concentrated under reduced pressure to obtain Example 774E as a mixture of diastereoisomers. LRMS calculated for C₁₆H₁₃N₂O₂F₂C₁Br: 382.0; found: 380.9 (M−H).

### Example 774F 4-amino-2′-bromo-5′,6′-difluorospiro[cyclohexane-1,1′-indene]-4-carboxylic acid

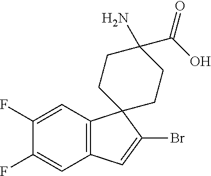 [SMILES: NC1(C(=O)O)CCC2(CC1)C(Br)=Cc1cc(F)c(F)cc12]

Using General procedure 15 and Example 774E as the appropriate hydantoin, Example 774F was obtained as a mixture of diastereoisomers. ¹H NMR (500 MHz, DMSO-d₆) δ ppm: 8.08 (dd, 1H), 7.43 (dd 1H), 7.00 (s, 1H), 2.53 (m, 2H), 2.09 (td, 2H), 1.76 (d, 2H), 1.04 (d, 2H).

### Example 774G 2′-bromo-4-(3-chloroanilino)-5′,6′-difluorospiro[cyclohexane-1,1′-indene]-4-carboxylic acid

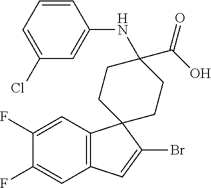 [SMILES: O=C(O)C1(Nc2cccc(Cl)c2)CCC2(CC1)C(Br)=Cc1cc(F)c(F)cc12]

Using General procedure 16 and Example 774E as the appropriate amino acid and 1-chloro-3-iodo-benzene as the appropriate iodobenzene, Example 774G was obtained as a mixture of diastereoisomers. LRMS calculated for C₂₁H₁₇NO₂F₂ClBr: 467; found: 468 (M+H).

### Example 774H methyl (1s,4s)-2′-bromo-4-(3-chloroanilino)-5′,6′-difluorospiro[cyclohexane-1,1′-indene]-4-carboxylate

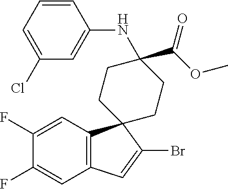 [SMILES: COC(=O)[C@]1(Nc2cccc(Cl)c2)CC[C@]2(CC1)C(Br)=Cc1cc(F)c(F)cc12]

Using General procedure 17b and Example 774G as the appropriate amino acid, a mixture of diastereoisomers was obtained. DCM and hexane were added and the formed precipitate was filtered to obtain Example 774H. ¹H NMR (500 MHz, DMSO-d₆) δ ppm: 7.71 (dd, 1H), 7.46 (dd, 1H), 7.10 (t, 1H), 7.01 (s, 1H), 6.62 (t, 1H), 6.60 (dm, 1H), 6.49 (dm, 1H), 6.48 (s, 1H), 3.69 (s, 3H), 2.38-2.23 (m, 4H), 2.19/1.00 (m+m, 4H). HRMS calculated for C₂₂H₁₉NO₂F₂ClBr: 481.0256; found: 482.0319 (M+H).

### Example 774I methyl (1r,4R)-4-(3-chloroanilino)-5′,6′-difluoro-2′-{(2R)-3-[(4-methoxyphenyl)methoxy]-2-methylpropyl}spiro[cyclohexane-1,1′-indene]-4-carboxylate

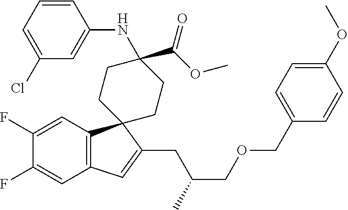 [SMILES: COc1ccc(COC[C@H](C)CC2=Cc3cc(F)c(F)cc3[C@]23CC[C@@](Nc2cccc(Cl)c2)(C(=O)OC)CC3)cc1]

Using General procedure 27b and Example 774H as the appropriate 2-bromo-indene derivative and Preparation 3d as the appropriate Zn reagent, Example 774I was obtained. ¹H NMR (500 MHz, DMSO-d₆) δ ppm: 7.58 (dd, 1H), 7.31 (dd, 1H), 7.21 (d, 2H), 7.09 (t, 1H), 6.85 (d, 2H), 6.64 (dd, 1H), 6.60 (dd, 1H), 6.48 (dd, 1H), 6.41 (m, 2H), 4.39/4.36 (d+d, 2H), 3.72 (s, 3H), 3.69 (s, 3H), 3.32/3.27 (dd+dd, 2H), 2.34/1.98 (dd+dd, 2H), 2.33-0.84 (m, 8H), 2.13 (m, 1H), 0.92 (d, 3H). HRMS calculated for C₃₄H₃₆NO₄F₂Cl: 595.2301; found: 596.2371 (M+H).

### Example 774J methyl (1r,4R)-4-(3-chloroanilino)-5′,6′-difluoro-2′-[(2R)-3-hydroxy-2-methylpropyl]spiro[cyclohexane-1,1′-indene]-4-carboxylate

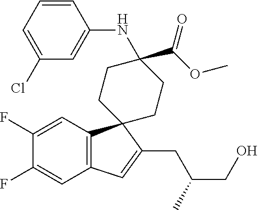 [SMILES: COC(=O)[C@]1(Nc2cccc(Cl)c2)CC[C@]2(CC1)C(C[C@@H](C)CO)=Cc1cc(F)c(F)cc12]

Using General procedure 28b and Example 774I as the appropriate PMB derivative, Example 774J was obtained. ¹H NMR (500 MHz, DMSO-d₆) δ ppm: 7.58 (dd, 1H), 7.34 (dd, 1H), 7.09 (t, 1H), 6.63 (t, 1H), 6.59 (dm, 1H), 6.48 (dm, 1H), 6.43 (t, 1H), 6.41 (s, 1H), 4.56 (t, 1H), 3.69 (s, 3H), 3.32/3.29 (m+m, 2H), 2.35/1.90 (m+m, 2H), 2.31/2.21 (m+m, 4H), 2.10/0.88 (m+m, 4H), 1.94 (m, 1H), 0.88 (d, 3H). HRMS calculated for C₂₆H₂₈NO₃F₂Cl: 475.1726; found: 476.1790 (M+H).

### Example 774 (1r,4R)-4-(3-chloroanilino)-5′,6′-difluoro-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

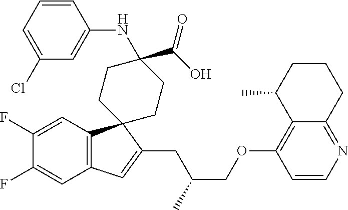 [SMILES: C[C@@H](COc1ccnc2c1[C@H](C)CCC2)CC1=Cc2cc(F)c(F)cc2[C@]12CC[C@@](Nc1cccc(Cl)c1)(C(=O)O)CC2]

Using General procedure 32 and Example 774J as the appropriate indene and Preparation 2a1 as the appropriate alcohol, Example 774 was obtained. ¹H NMR (500 MHz, DMSO-d₆) δ ppm: 12.87 (br s, 1H), 8.13 (d, 1H), 7.57 (dd, 1H), 7.35 (dd, 1H), 7.07 (t, 1H), 6.76 (d, 1H), 6.62 (dd, 1H), 6.57 (dd, 1H), 6.54 (dd, 1H), 6.52 (s, 1H), 6.35 (br s, 1H), 4.02/3.93 (dd+dd, 2H), 3.06 (m, 1H), 2.75/2.63 (dd+dd, 2H), 2.48/2.21 (dd+dd, 2H), 2.41 (dd, 1H), 2.34-0.82 (m, 8H), 1.79/1.69 (m+m, 2H), 1.59/1.54 (m+m, 2H), 1.11 (d, 3H), 1.09 (d, 3H). HRMS calculated for C₃₅H₃₇N₂O₃F₂Cl: 606.2461; found: 607.2528 (M+H).

### Example 775 and Example 776

### Example 775A methyl (1r,2′S,4S)-4-(3-chloroanilino)-5′,6′-difluoro-2′-[(2R)-3-hydroxy-2-methylpropyl]-2′,3′-dihydrospiro[cyclohexane-1,1′-indene]-4-carboxylate

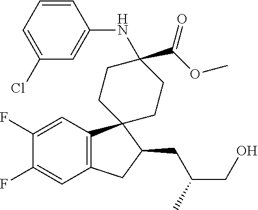 [SMILES: COC(=O)[C@]1(Nc2cccc(Cl)c2)CC[C@@]2(CC1)c1cc(F)c(F)cc1C[C@@H]2C[C@@H](C)CO]

and

### Example 775B methyl (1r,2′R,4R)-4-(3-chloroanilino)-5′,6′-difluoro-2′-[(2R)-3-hydroxy-2-methylpropyl]-2′,3′-dihydrospiro[cyclohexane-1,1′-indene]-4-carboxylate

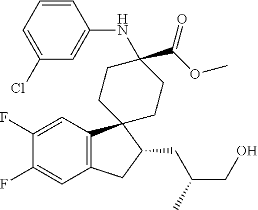 [SMILES: COC(=O)[C@]1(Nc2cccc(Cl)c2)CC[C@@]2(CC1)c1cc(F)c(F)cc1C[C@H]2C[C@@H](C)CO]

Using General procedure 19 and Example 774J as the appropriate indene, a mixture of diastereoisomers was obtained. The diastereoisomers were separated by prep RP-HPLC using 25 mM aq. NH₄HCO₃ solution and MeCN as eluents. The diastereoisomer eluting earlier was collected as Example 775A. ¹H NMR (500 MHz, DMSO-d₆) δ ppm: 7.32 (dd, 1H), 7.27 (dd, 1H), 7.06 (t, 1H), 6.59 (t, 1H), 6.57 (dm, 1H), 6.45 (dm, 1H), 6.30 (s, 1H), 4.46 (t, 1H), 3.65 (s, 3H), 3.22/3.20 (m+m, 2H), 2.92/2.47 (dd+dd, 2H), 2.48-1.28 (m, 8H), 2.13 (m, 1H), 1.53 (m, 1H), 1.32/1.04 (m+m, 2H), 0.85 (d, 3H). HRMS calculated for C₂₆H₃₀NO₃F₂Cl: 477.1882; found: 478.1950 (M+H).

The diastereoisomer eluting later was collected as Example 775B. ¹H NMR (500 MHz, DMSO-d₆) δ ppm: 7.32 (dd, 1H), 7.27 (dd, 1H), 7.06 (t, 1H), 6.59 (t, 1H), 6.57 (dm, 1H), 6.45 (dm, 1H), 6.29 (s, 1H), 4.40 (t, 1H), 3.65 (s, 3H), 3.40/3.20 (m+m, 2H), 2.95/2.52 (dd+dd, 2H), 2.47-1.24 (m, 8H), 2.15 (m, 1H), 1.58 (m, 1H), 1.43/0.99 (m+m, 2H), 0.90 (d, 3H). HRMS calculated for C₂₆H₃₀NO₃F₂Cl: 477.1882; found: 478.1955 (M+H).

### Example 775 (1r,2′S,4S)-4-(3-chloroanilino)-5′,6′-difluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

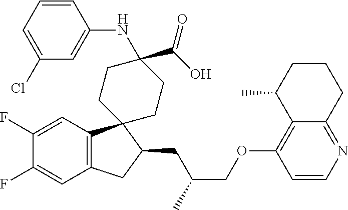 [SMILES: C[C@@H](COc1ccnc2c1[C@H](C)CCC2)C[C@H]1Cc2cc(F)c(F)cc2[C@]12CC[C@@](Nc1cccc(Cl)c1)(C(=O)O)CC2]

Using General procedure 32 and Example 775A as the appropriate indene and Preparation 2a1 as the appropriate alcohol, Example 775 was obtained. ¹H NMR (500 MHz, DMSO-d₆) δ ppm: 12.68 (br s, 1H), 8.14 (d, 1H), 7.33-7.24 (m, 2H), 7.04 (t, 1H), 6.76 (d, 1H), 6.61 (t, 1H), 6.54 (dm, 1H), 6.53 (dm, 1H), 6.27 (br s, 1H), 3.88/3.85 (dd+dd, 2H), 3.03 (m, 1H), 2.99/2.51 (dd+dd, 2H), 2.76/2.65 (m+m, 2H), 2.47-1.33 (m, 12H), 2.20 (m, 1H), 1.97 (m, 1H), 1.46/1.31 (m+m, 2H), 1.04 (d, 3H), 1.02 (d, 3H). HRMS calculated for C₃₅H₃₉N₂O₃F₂Cl: 608.2617 found: 609.2685 (M+H).

### Example 776 (1r,2′R,4R)-4-(3-chloroanilino)-5′,6′-difluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

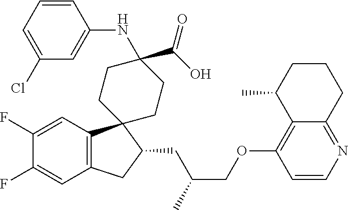 [SMILES: C[C@@H](COc1ccnc2c1[C@H](C)CCC2)C[C@@H]1Cc2cc(F)c(F)cc2[C@]12CC[C@@](Nc1cccc(Cl)c1)(C(=O)O)CC2]

Using General procedure 32 and Example 775B as the appropriate indene and Preparation 2a1 as the appropriate alcohol, Example 776 was obtained. ¹H NMR (500 MHz, DMSO-d₆) δ ppm: 12.72 (br s, 1H), 8.13 (d, 1H), 7.31 (dd, 1H), 7.28 (dd, 1H), 7.03 (t, 1H), 6.77 (d, 1H), 6.59 (t, 1H), 6.54 (dm, 1H), 6.49 (dm, 1H), 6.16 (br s, 1H), 4/3.86 (dd+dd, 2H), 3.00 (m, 1H), 2.99/2.57 (dd+dd, 2H), 2.73/2.60 (m+m, 2H), 2.42-1.24 (m, 12H), 2.14 (m, 1H), 2.05 (m, 1H), 1.68/1.23 (m+m, 2H), 1.09 (d, 3H), 1.08 (d, 3H). HRMS calculated for C₃₅H₃₉N₂O₃F₂Cl: 608.2617; found: 609.2681 (M+H).

### Example 777

### Example 777A methyl (1s,4s)-2′-bromo-5′-chloro-4-(3-chloroanilino)spiro[cyclohexane-1,1′-indene]-4-carboxylate

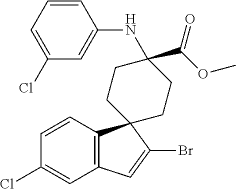 [SMILES: COC(=O)[C@]1(Nc2cccc(Cl)c2)CC[C@]2(CC1)C(Br)=Cc1cc(Cl)ccc12]

Using General procedure 17a and Example 135 as the appropriate amino acid, Example 777A was obtained. ¹H NMR (500 MHz, DMSO-d₆) δ ppm: 7.69 (d, 1H), 7.45 (d, 1H), 7.29 (dd, 1H), 7.09 (t, 1H), 7.04 (s, 1H), 6.61 (m, 1H), 6.60 (dm, 1H), 6.50 (br, 1H), 6.48 (dm, 1H), 3.68 (s, 3H), 2.40-2.24 (m, 4H), 2.20/0.99 (td+br d, 4H). HRMS calculated for C₂₂H₂₀BrCl₂NO₂: 479.0055; found 480.0122 (M+H).

### Example 777B methyl (1r,4R)-5′-chloro-4-(3-chloroanilino)-2′-{(2R)-3-[(4-methoxyphenyl)methoxy]-2-methylpropyl}spiro[cyclohexane-1,1′-indene]-4-carboxylate

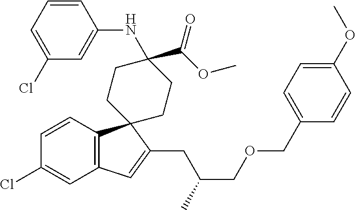 [SMILES: COc1ccc(COC[C@H](C)CC2=Cc3cc(Cl)ccc3[C@]23CC[C@@](Nc2cccc(Cl)c2)(C(=O)OC)CC3)cc1]

Using General procedure 27b and Example 777A as the appropriate 2-bromo-indene derivative and Preparation 3d as the appropriate Zn reagent, Example 777B was obtained. ¹H NMR (500 MHz, DMSO-d₆) δ ppm: 7.61 (d, 1H), 7.32 (d, 1H), 7.21 (d, 2H), 7.15 (dd, 1H), 7.09 (t, 1H), 6.86 (d, 2H), 6.64 (t, 1H), 6.60 (dd, 1H), 6.48 (dd, 1H), 6.44 (s, 1H), 6.40 (s, 1H), 4.39 (d, 1H), 4.37 (d, 1H), 3.73 (s, 3H), 3.68 (s, 3H), 3.36-3.24 (m, 2H), 2.40-1.95 (m, 9H), 0.94 (d, 3H), 0.90-0.81 (m, 2H).

### Example 777C methyl (1r,4R)-5′-chloro-4-(3-chloroanilino)-2′-[(2R)-3-hydroxy-2-methylpropyl]spiro[cyclohexane-1,1′-indene]-4-carboxylate

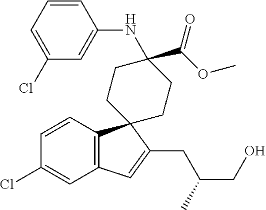 [SMILES: COC(=O)[C@]1(Nc2cccc(Cl)c2)CC[C@]2(CC1)C(C[C@@H](C)CO)=Cc1cc(Cl)ccc12]

Using General procedure 28b and Example 777B as the appropriate PMB derivative, Example 777C was obtained. ¹H NMR (500 MHz, DMSO-d₆) δ ppm: 7.61 (d, 1H), 7.35 (d, 1H), 7.15 (dd, 2H), 7.09 (t, 1H), 6.63 (t, 1H), 6.59 (dd, 1H), 6.48 (dd, 1H), 6.47 (s, 1H), 6.41 (s, 1H), 3.68 (s, 3H), 3.38-3.25 (m, 2H), 2.42-1.84 (m, 9H), 0.89 (d, 3H), 0.95-0.80 (m, 2H).

### Example 777 (1r,4R)-5′-chloro-4-(3-chloroanilino)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

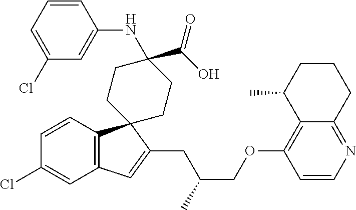 [SMILES: C[C@@H](COc1ccnc2c1[C@H](C)CCC2)CC1=Cc2cc(Cl)ccc2[C@]12CC[C@@](Nc1cccc(Cl)c1)(C(=O)O)CC2]

Using General procedure 32 and Example 777C as the appropriate indene and Preparation 2a1 as the appropriate alcohol, Example 777 was obtained. ¹H NMR (500 MHz, DMSO-d₆) δ ppm: 12.84 (br s, 1H), 8.14 (d, 1H), 7.63 (d, 1H), 7.36 (d, 1H), 7.17 (dd, 1H), 7.07 (t, 1H), 6.77 (d, 1H), 6.63 (t, 1H), 6.57 (dd, 1H), 6.55 (m, 2H), 6.35 (br s, 1H), 4.02/3.93 (dd+dd, 2H), 3.06 (m, 1H), 2.76/2.64 (m+m, 2H), 2.49/2.22 (m+m, 2H), 2.41 (m, 1H), 2.32-0.86 (m, 8H), 1.79/1.70 (m+m, 2H), 1.59/1.55 (m+m, 2H), 1.12 (d, 3H), 1.10 (d, 3H). HRMS calculated for C₃₅H₃₈N₂O₃Cl₂: 604.2260; found: 605.2336 (M+H).

### Example 778 and Example 779

### Example 778A methyl (1r,2′S,4S)-5′-chloro-4-(3-chloroanilino)-2′-[(2R)-3-hydroxy-2-methylpropyl]-2′,3′-dihydrospiro[cyclohexane-1,1′-indene]-4-carboxylate

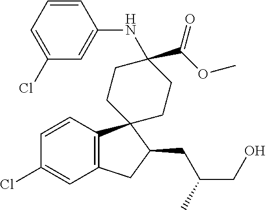 [SMILES: COC(=O)[C@]1(Nc2cccc(Cl)c2)CC[C@@]2(CC1)c1ccc(Cl)cc1C[C@@H]2C[C@@H](C)CO]

and

### Example 778B methyl (1r,2′R,4R)-5′-chloro-4-(3-chloroanilino)-2′-[(2R)-3-hydroxy-2-methylpropyl]-2′,3′-dihydrospiro[cyclohexane-1,1′-indene]-4-carboxylate

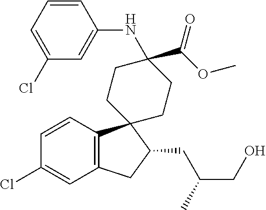 [SMILES: COC(=O)[C@]1(Nc2cccc(Cl)c2)CC[C@@]2(CC1)c1ccc(Cl)cc1C[C@H]2C[C@@H](C)CO]

Using General procedure 19 and Example 777C as the appropriate indene, a mixture of diastereoisomers was obtained. The diastereoisomers were separated by prep RP-HPLC using 25 mM aq. NH₄HCO₃ solution and MeCN as eluents. The diastereoisomer eluting earlier was collected as Example 778A. ¹H NMR (500 MHz, DMSO-d₆) δ ppm: 7.36 (d, 1H), 7.26 (d, 1H), 7.21 (dd, 1H), 7.06 (t, 1H), 6.58 (t, 1H), 6.56 (dm, 1H), 6.44 (dm, 1H), 6.31 (s, 1H), 4.46 (t, 1H), 3.64 (s, 3H), 3.21 (m, 2H), 2.94/2.51 (dd+dd, 2H), 2.47-1.27 (m, 8H), 2.10 (m, 1H), 1.54 (m, 1H), 1.33/1.05 (m+m, 2H), 0.84 (d, 3H). HRMS calculated for C₂₆H₃₁N₂O₃Cl₂: 475.1681; found: 476.1753 (M+H).

The diastereoisomer eluting later was collected as Example 778B. ¹H NMR (500 MHz, DMSO-d₆) δ ppm: 7.36 (d, 1H), 7.27 (d, 1H), 7.20 (dd, 1H), 7.06 (t, 1H), 6.58 (t, 1H), 6.57 (dm, 1H), 6.45 (dm, 1H), 6.30 (s, 1H), 4.40 (m, 1H), 3.64 (s, 3H), 3.40/3.20 (dd+dd, 2H), 2.97/2.55 (dd+dd, 2H), 2.45-1.24 (m, 8H), 2.10 (m, 1H), 1.59 (m, 1H), 1.44/1.01 (m+td, 2H), 0.90 (d, 3H). HRMS calculated for C₂₆H₃₁N₂O₃Cl₂: 475.1681; found: 476.1754 (M+H).

### Example 778 (1r,2′S,4S)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

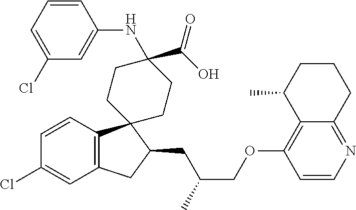 [SMILES: C[C@@H](COc1ccnc2c1[C@H](C)CCC2)C[C@H]1Cc2cc(Cl)ccc2[C@]12CC[C@@](Nc1cccc(Cl)c1)(C(=O)O)CC2]

Using General procedure 32 and Example 778A as the appropriate indene and Preparation 2a1 as the appropriate alcohol, Example 778 was obtained. ¹H NMR (500 MHz, DMSO-d₆) δ ppm: 12.71 (br s, 1H), 8.14 (d, 1H), 7.35 (d, 1H), 7.26 (d, 1H), 7.22 (dd, 1H), 7.04 (t, 1H), 6.76 (d, 1H), 6.61 (t, 1H), 6.54 (dm, 1H), 6.53 (dm, 1H), 6.25 (br s, 1H), 3.88/3.86 (dd+dd, 2H), 3.03 (m, 1H), 3.02/2.54 (dd+dd, 2H), 2.76/2.65 (m+m, 2H), 2.44-1.34 (m, 12H), 2.17 (m, 1H), 1.98 (m, 1H), 1.47/1.32 (m+m, 2H), 1.04 (d, 3H), 1.01 (d, 3H). HRMS calculated for C₃₅H₄₀N₂O₃Cl₂: 606.2416; found: 607.2484 (M+H).

### Example 779 (1r,2′R,4R)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

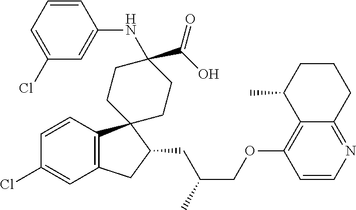 [SMILES: C[C@@H](COc1ccnc2c1[C@H](C)CCC2)C[C@@H]1Cc2cc(Cl)ccc2[C@]12CC[C@@](Nc1cccc(Cl)c1)(C(=O)O)CC2]

Using General procedure 32 and Example 778B as the appropriate indene and Preparation 2a1 as the appropriate alcohol, Example 779 was obtained. ¹H NMR (500 MHz, DMSO-d₆) δ ppm: 12.69 (br s, 1H), 8.13 (d, 1H), 7.38 (d, 1H), 7.26 (d, 1H), 7.21 (dd, 1H), 7.03 (t, 1H), 6.77 (d, 1H), 6.60 (t, 1H), 6.54 (dm, 1H), 6.49 (dm, 1H), 6.15 (br s, 1H), 4.00/3.86 (dd+dd, 2H), 3.01/2.59 (dd+dd, 2H), 3.00 (m, 1H), 2.73/2.6 (m+m, 2H), 2.44-1.25 (m, 12H), 2.10 (m, 1H), 2.06 (m, 1H), 1.69/1.23 (m+m, 2H), 1.10 (d, 3H), 1.08 (d, 3H). HRMS calculated for C₃₅H₄₀N₂O₃Cl₂: 606.2416; found: 607.2502 (M+H).

### Example 780 (1r,2′S,4S)-4-(3-chloroanilino)-6′-hydroxy-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylic acid

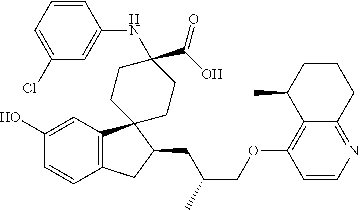 [SMILES: C[C@@H](COc1ccnc2c1[C@@H](C)CCC2)C[C@H]1Cc2ccc(O)cc2[C@]12CC[C@@](Nc1cccc(Cl)c1)(C(=O)O)CC2]

Using General procedure 33a and Preparation 14b as the appropriate ester, Example 780 was obtained. ¹H NMR (500 MHz, DMSO-d₆) δ ppm: 12.69 (br s, 1H), 9.09 (s, 1H), 8.13 (d, 1H), 7.05 (t, 1H), 6.95 (d, 1H), 6.80 (d, 1H), 6.75 (d, 1H), 6.63 (t, 1H), 6.53 (m, 3H), 6.20 (br s, 1H), 3.91/3.82 (dd+dd, 2H), 3.02 (m, 1H), 2.87/2.42 (dd+dd, 2H), 2.76/2.64 (dm+m, 2H), 2.44-1.32 (m, 8H), 2.11 (m, 1H), 1.97 (m, 1H), 1.81/1.73 (m+m, 2H), 1.62 (m, 2H), 1.42/1.34 (m+m, 2H), 1.14 (d, 3H), 1.04 (d, 3H). HRMS calculated for C₃₅H₄₁N₂O₄Cl: 588.2755; found: 589.2828 (M+H).

### Example 781 (1r,2′S,4S)-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

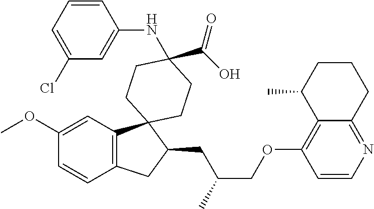 [SMILES: COc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 14a as the appropriate indane and MeOH as the appropriate alcohol, Example 781 was obtained. ¹H NMR (500 MHz, DMSO-d₆) δ ppm: 8.14 (d, 1H), 7.10 (d, 1H), 7.04 (t, 1H), 6.90 (d, 1H), 6.77 (d, 1H), 6.73 (dd, 1H), 6.60 (t, 1H), 6.53 (dm, 1H), 6.52 (dm, 1H), 3.90/3.84 (dd+dd, 2H), 3.90/3.84 (dd+dd, 2H), 3.72 (s, 3H), 3.05 (m, 1H), 2.76/2.65 (dm+m, 2H), 2.48-1.32 (m, 8H), 2.14 (m, 1H), 1.98 (m, 1H), 1.82-1.66 (m, 2H), 1.71-1.56 (m, 2H), 1.48/1.33 (m+m, 2H), 1.06 (d, 3H), 1.04 (d, 3H). HRMS calculated for C₃₆H₄₃N₂O₄Cl: 602.2911; found: 603.2999 (M+H).

### Example 782 (1r,2′R,4R)-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

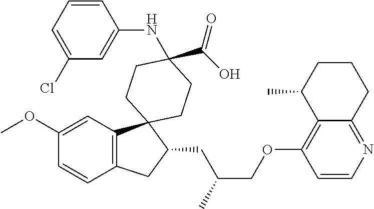 [SMILES: COc1ccc2c(c1)[C@]1(CC[C@@](Nc3cccc(Cl)c3)(C(=O)O)CC1)[C@H](C[C@@H](C)COc1ccnc3c1[C@H](C)CCC3)C2]

Using General procedure 32 and Preparation 14c as the appropriate indane and MeOH as the appropriate alcohol, Example 782 was obtained as a white powder. LRMS calculated for C₃₆H₄₃ClN₂O₄: 602; found: 603 (M+H). ¹H NMR (400 MHz, DMSO-d₆) δ ppm: 8.14 (d, J=5.6 Hz, 1H), 7.11 (d, J=8.2 Hz, 1H), 7.03 (t, J=8.1 Hz, 1H), 6.94 (d, J=2.3 Hz, 1H), 6.78 (d, J=5.6 Hz, 1H), 6.73 (dd, J=8.2, 2.3 Hz, 1H), 6.61 (t, J=2.2 Hz, 1H), 6.57-6.47 (m, 2H), 6.12 (br s, 1H), 4.01 (dd, J=9.6, 4.3 Hz, 1H), 3.87 (dd, J=9.6, 5.4 Hz, 1H), 3.72 (s, 3H), 3.07-2.98 (m, 1H), 2.93 (dd, J=15.2, 7.1 Hz, 1H), 2.79-2.69 (m, 1H), 2.67-2.46 (m, 2H), 2.45-2.33 (m, 1H), 2.18-1.92 (m, 4H), 1.90-1.61 (m, 6H), 1.60-1.17 (m, 5H), 1.15-1.06 (m, 6H).

### Example 783 (1r,2′S,4S)-4-(3-chloroanilino)-6′-methoxy-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylic acid

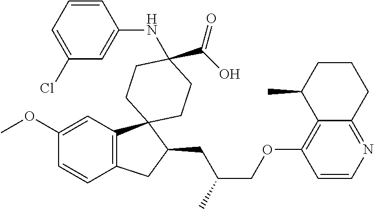 [SMILES: COc1ccc2c(c1)[C@]1(CC[C@@](Nc3cccc(Cl)c3)(C(=O)O)CC1)[C@@H](C[C@@H](C)COc1ccnc3c1[C@@H](C)CCC3)C2]

Using General procedure 32 and Preparation 14b as the appropriate indane and MeOH as the appropriate alcohol, Example 781 was obtained. ¹H NMR (500 MHz, DMSO-d₆) δ ppm: 12.69 (br s, 1H), 8.14 (d, 1H), 7.10 (d, 1H), 7.04 (t, 1H), 6.89 (d, 1H), 6.75 (d, 1H), 6.72 (dd, 1H), 6.62 (t, 1H), 6.54 (dm, 1H), 6.51 (dm, 1H), 6.22 (br s, 1H), 3.91/3.83 (dd+dd, 2H), 3.71 (s, 3H), 3.01 (m, 1H), 2.93/2.45 (dd+dd, 2H), 2.76/2.64 (m+m, 2H), 2.50-1.30 (m, 12H), 2.14 (m, 1H), 1.98 (m, 1H), 1.41/1.35 (m+m, 2H), 1.14 (d, 3H), 1.04 (d, 3H). HRMS calculated for C₃₆H₄₃N₂O₄Cl: 602.2911; found: 603.2979 (M+H).

### Example 784 (1r,2′S,4S)-4-(3-chloroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

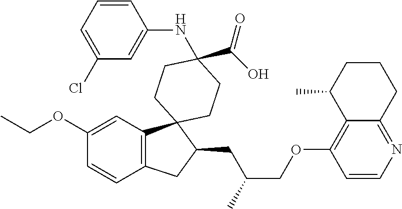 [SMILES: CCOc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 14a as the appropriate indane and EtOH as the appropriate alcohol, Example 784 was obtained. ¹H NMR (500 MHz, DMSO-d₆) δ ppm: 8.14 (d, 1H), 7.08 (d, 1H), 7.04 (t, 1H), 6.89 (d, 1H), 6.77 (d, 1H), 6.71 (dd, 1H), 6.60 (t, 1H), 6.54 (dm, 1H), 6.52 (dm, 1H), 6.23 (br s, 1H), 3.97 (m, 2H), 3.90/3.84 (dd+dd, 2H), 3.05 (m, 1H), 2.92/2.44 (dd+dd, 2H), 2.76/2.65 (dm+m, 2H), 2.46-1.29 (m, 14H), 2.12 (m, 1H), 1.98 (m, 1H), 1.32 (t, 3H), 1.06 (d, 3H), 1.04 (d, 3H). HRMS calculated for C₃₇H₄₅N₂O₄Cl: 616.3068; found: 617.3162 (M+H).

### Example 785

### Example 785A (1s,4s)-2′-bromo-4-(3-chloro-4-fluoroanilino)spiro[cyclohexane-1,1′-indene]-4-carboxylic acid

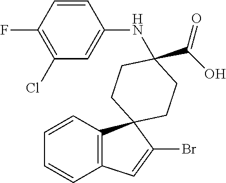 [SMILES: O=C(O)[C@]1(Nc2ccc(F)c(Cl)c2)CC[C@]2(CC1)C(Br)=Cc1ccccc12]

Using General procedure 16 and Preparation 5a as the appropriate amino acid and 2-chloro-1-fluoro-4-iodobenzene as the appropriate iodobenzene, Example 785A was obtained. LRMS calculated for C₂₁H₁₈NO₂ClF: 449.0; found: 450.0 (M+H).

### Example 785B methyl (1s,4s)-2′-bromo-4-(3-chloro-4-fluoroanilino)spiro[cyclohexane-1,1′-indene]-4-carboxylate

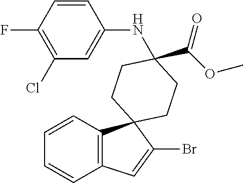 [SMILES: COC(=O)[C@]1(Nc2ccc(F)c(Cl)c2)CC[C@]2(CC1)C(Br)=Cc1ccccc12]

Using General procedure 17b and Example 785A as the appropriate amino acid, Example 785B was obtained. ¹H NMR (500 MHz, DMSO-d₆) δ ppm: 7.71 (dm, 1H), 7.37 (dm, 1H), 7.30 (m, 1H), 7.23 (m, 1H), 7.14 (t, 1H), 7.04 (s, 1H), 6.68 (dd, 1H), 6.49 (dm, 1H), 6.38 (s, 1H), 3.69 (s, 3H), 2.40/2.25 (m+m, 4H), 2.20/0.98 (m+m, 4H). HRMS calculated for C₂₂H₂₀NO₂BrClF: 463.0350; found: 464.0425 (M+H).

### Example 785C methyl (1s,4s)-2′-bromo-4-[(3-chloro-4-fluorophenyl)(trifluoroacetyl)amino]spiro[cyclohexane-1,1′-indene]-4-carboxylate

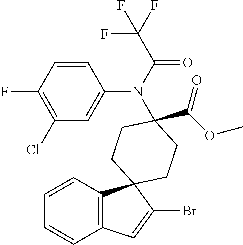 [SMILES: COC(=O)[C@]1(N(C(=O)C(F)(F)F)c2ccc(F)c(Cl)c2)CC[C@]2(CC1)C(Br)=Cc1ccccc12]

Using General procedure 22 and Example 785B as the appropriate indene, Example 785C was obtained. ¹H NMR (500 MHz, DMSO-d₆) δ ppm: 8.01 (dd, 1H), 7.74 (dm, 1H), 7.67 (d, 1H), 7.49 (dm, 1H), 7.32 (dm, 1H), 7.27 (m, 1H), 7.23 (m, 1H), 7.03 (s, 1H), 3.83 (s, 3H), 2.56-1.36 (m, 8H). HRMS calculated for C₂₄H₁₉NO₃BrClF₄: 559.0173; found: 559.0175 (M).

### Example 785D methyl (1s,4s)-6′-acetyl-2′-bromo-4-[(3-chloro-4-fluorophenyl)(trifluoroacetyl)amino]spiro[cyclohexane-1,1′-indene]-4-carboxylate

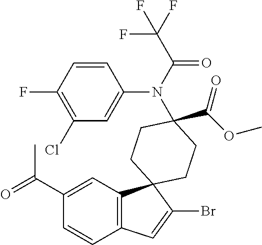 [SMILES: COC(=O)[C@]1(N(C(=O)C(F)(F)F)c2ccc(F)c(Cl)c2)CC[C@]2(CC1)C(Br)=Cc1ccc(C(C)=O)cc12]

Using General procedure 23 and Example 785C as the appropriate indene, Example 785D was obtained. ¹H NMR (500 MHz, DMSO-d₆) δ ppm: 8.10 (d, 1H), 8.03 (dd, 1H), 7.94 (dd, 1H), 7.76 (dm, 1H), 7.68 (t, 1H), 7.47 (d, 1H), 7.16 (s, 1H), 3.86 (s, 3H), 2.60 (s, 3H), 2.60-1.34 (m, 8H). HRMS calculated for C₂₆H₂₁NO₄BrClF₄: 601.0278; found: 602.0353 (M+H).

### Example 785E methyl (1s,4s)-6′-(acetyloxy)-2′-bromo-4-[(3-chloro-4-fluorophenyl)(trifluoroacetyl)amino]spiro[cyclohexane-1,1′-indene]-4-carboxylate

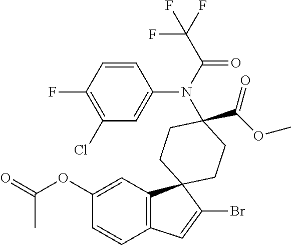 [SMILES: COC(=O)[C@]1(N(C(=O)C(F)(F)F)c2ccc(F)c(Cl)c2)CC[C@]2(CC1)C(Br)=Cc1ccc(OC(C)=O)cc12]

Using General procedure 24 and Example 785D as the appropriate indene, Example 785E was obtained. ¹H NMR (500 MHz, DMSO-d₆) δ ppm: 7.99 (dd, 1H), 7.72 (dm, 1H), 7.67 (t, 1H), 7.34 (d, 1H), 7.17 (d, 1H), 7.05 (dd, 1H), 7.03 (s, 1H), 3.82 (s, 3H), 2.50-1.42 (m, 8H), 2.29 (s, 3H). HRMS calculated for C₂₆H₂₁NO₅BrClF₄: 617.0228; found: 635.0571 (M+NH₄).

### Example 785F methyl (1s,4s)-2′-bromo-4-[(3-chloro-4-fluorophenyl)(trifluoroacetyl)amino]-6′-hydroxyspiro[cyclohexane-1,1′-indene]-4-carboxylate

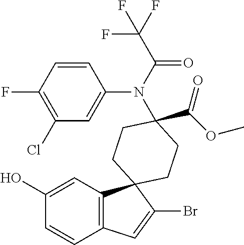 [SMILES: COC(=O)[C@]1(N(C(=O)C(F)(F)F)c2ccc(F)c(Cl)c2)CC[C@]2(CC1)C(Br)=Cc1ccc(O)cc12]

Using General procedure 25 and Example 785E as the appropriate indene, Example 785F was obtained. ¹H NMR (500 MHz, DMSO-d₆) δ ppm: 9.53 (s, 1H), 7.98 (dd, 1H), 7.71 (m, 1H), 7.66 (t, 1H), 7.09 (d, 1H), 6.92 (d, 1H), 6.87 (s, 1H), 6.65 (dd, 1H), 3.83 (s, 3H), 2.48-1.43 (m, 8H). HRMS calculated for C₂₄H₁₉NO₄BrClF₄: 575.0122; found: 593.0453 (M+NH₄).

### Example 785G methyl (1s,4s)-2′-bromo-4-[(3-chloro-4-fluorophenyl)(trifluoroacetyl)amino]-6′-ethoxyspiro[cyclohexane-1,1′-indene]-4-carboxylate

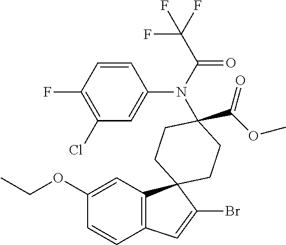 [SMILES: CCOc1ccc2c(c1)[C@]1(CC[C@](C(=O)OC)(N(C(=O)C(F)(F)F)c3ccc(F)c(Cl)c3)CC1)C(Br)=C2]

Using General procedure 30a and Example 785F as the appropriate indene and EtOH as the appropriate alcohol, Example 785G was obtained. ¹H NMR (500 MHz, DMSO-d₆) δ ppm: 8.00 (dd, 1H), 7.73 (dm, 1H), 7.67 (t, 1H), 7.22 (d, 1H), 7.04 (d, 1H), 6.93 (s, 1H), 6.83 (dd, 1H), 4.04 (q, 2H), 3.83 (s, 3H), 2.55-1.31 (m, 8H), 1.35 (t, 3H). HRMS calculated for C₂₆H₂₃NO₄BrClF₄: 603.0435; found: 621.0780 (M+NH₄).

### Example 785H methyl (1r,4R)-4-[(3-chloro-4-fluorophenyl)(trifluoroacetyl)amino]-6′-ethoxy-2′-{(2R)-3-[(4-methoxyphenyl)methoxy]-2-methylpropyl}spiro[cyclohexane-1,1′-indene]-4-carboxylate

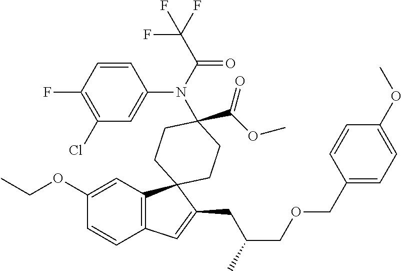

Using General procedure 27b and Example 785G as the appropriate 2-bromo-indene derivative and Preparation 3d as the appropriate Zn reagent, Example 785H was obtained. ¹H NMR (400 MHz, DMSO-d₆) δ ppm: 8.08/7.99 (dd, 1H), 7.76/7.70 (dm, 1H), 7.59/7.53 (t, 1H), 7.24/7.22 (m, 2H), 7.11 (d, 1H), 7.07 (d, 1H), 6.88 (m, 2H), 6.77 (dd, 1H), 6.32/6.31 (br s, 1H), 4.47-4.30 (d+d, 2H), 4.01 (q, 2H), 3.82 (s, 3H), 3.73 (s, 3H), 3.40-3.23 (m, 2H), 2.70-0.96 (m, 11H), 1.34 (s, 3H), 0.94/0.91 (d, 3H). HRMS calculated for C₃₈H₄₀NO₆ClF₄: 717.2480; found: 735.2798 (M+NH₄).

### Example 7851 methyl (1r,2′S,4S)-4-[(3-chloro-4-fluorophenyl)(trifluoroacetyl)amino]-6′-ethoxy-2′-{(2R)-3-[(4-methoxyphenyl)methoxy]-2-methylpropyl}-2′,3′-dihydrospiro[cyclohexane-1,1′-indene]-4-carboxylate

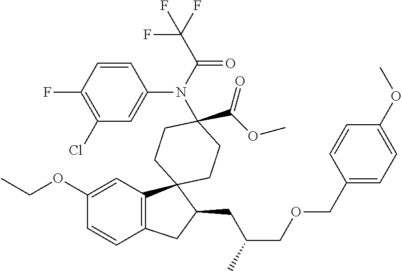 [SMILES: CCOc1ccc2c(c1)[C@]1(CC[C@](C(=O)OC)(N(C(=O)C(F)(F)F)c3ccc(F)c(Cl)c3)CC1)[C@@H](C[C@@H](C)COCc1ccc(OC)cc1)C2]

Using General procedure 19 and Example 785H as the appropriate indene, a mixture of diastereoisomers was obtained. The diastereoisomers were separated via flash chromatography using hexane and EtOAc as eluents. The diastereoisomer eluting later was collected as Example 7851. ¹H NMR (500 MHz, DMSO-d₆) δ ppm: 8.02-7.45 (m, 3H), 7.16/7.13 (m, 2H), 7.03 (d, 1H), 6.86/6.85 (m, 2H), 6.67 (dd, 1H), 6.54/6.53 (d, 1H), 4.31/4.28 (s, 2H), 3.95 (q, 2H), 3.78 (s, 3H), 3.72 (s, 3H), 3.20-2.94 (m, 2H), 2.87/2.38 (dd+dd, 2H), 2.50-0.57 (m, 11H), 2.22/2.16 (m, 1H), 1.29 (t, 3H), 0.77/0.73 (d, 3H). HRMS calculated for C₃₈H₄₂NO₆ClF₄: 719.1637; found: 737.2993 (M+NH₄).

### Example 785J methyl (1r,2′S,4S)-4-[(3-chloro-4-fluorophenyl)(trifluoroacetyl)amino]-6′-ethoxy-2′-[(2R)-3-hydroxy-2-methylpropyl]-2′,3′-dihydrospiro[cyclohexane-1,1′-indene]-4-carboxylate

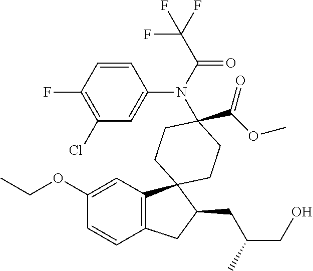 [SMILES: CCOc1ccc2c(c1)[C@]1(CC[C@](C(=O)OC)(N(C(=O)C(F)(F)F)c3ccc(F)c(Cl)c3)CC1)[C@@H](C[C@@H](C)CO)C2]

Using General procedure 28b and Example 7851 as the appropriate PMB derivative, Example 785J was obtained. ¹H NMR (500 MHz, DMSO-d₆) δ ppm: 7.98/7.81 (dd/dd, 1H), 7.61/7.51 (t/t, 1H), 7.61/7.52 (m/m, 1H), 7.03 (d, 1H), 6.65 (dm, 1H), 6.55/6.54 (d/d, 1H), 4.37/4.34 (t/t, 1H), 3.95 (q, 2H), 3.79/3.78 (s/s, 3H), 3.18-2.96 (m, 2H), 2.88/2.78/2.38 (dd/dd+d, 2H), 2.47-0.53 (m, 10H), 2.22/2.16 (m/m, 1H), 1.45 (m, 1H), 1.30/1.29 (t/t, 3H), 0.73/0.69 (d/d, 3H). HRMS calculated for C₃₀H₃₄NO₅ClF₄: 599.2062; found: 617.2399 (M+NH₄).

### Example 785 (1r,2′S,4S)-4-(3-chloro-4-fluoroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

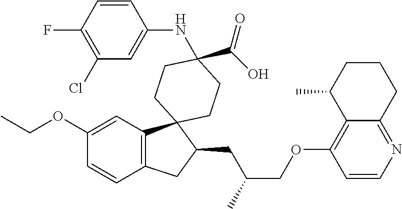 [SMILES: CCOc1ccc2c(c1)[C@]1(CC[C@@](Nc3ccc(F)c(Cl)c3)(C(=O)O)CC1)[C@@H](C[C@@H](C)COc1ccnc3c1[C@H](C)CCC3)C2]

Using General procedure 32 and Example 785J as the appropriate indane and Preparation 2a1 as the appropriate alcohol, Example 785 was obtained. ¹H NMR (500 MHz, DMSO-d₆) δ ppm: 12.66 (br s, 1H), 8.14 (d, 1H), 7.09 (t, 1H), 7.08 (d, 1H), 6.87 (d, 1H), 6.77 (d, 1H), 6.71 (dd, 1H), 6.67 (dd, 1H), 6.53 (dm, 1H), 6.14 (br s, 1H), 3.97 (q, 2H), 3.89/3.85 (dd+dd, 2H), 3.04 (m, 1H), 2.91/2.44 (dd+dd, 2H), 2.76/2.65 (m+m, 2H), 2.50-1.26 (m, 12H), 2.12 (m, 1H), 1.98 (m, 1H), 1.48/1.32 (m+m, 2H), 1.32 (t, 3H), 1.05 (d, 3H), 1.03 (d, 3H). HRMS calculated for C₃₇H₄₄N₂O₄FCl: 634.2974; found: 635.3045 (M+H).

### Example 786 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-[(propan-2-yl)oxy]-2′,3′-dihydrospiro[cyclohexane-1,1′-indene]-4-carboxylic acid

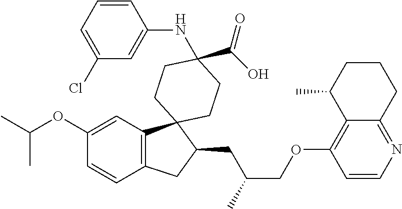 [SMILES: CC(C)Oc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 14a as the appropriate indane and iPrOH as the appropriate alcohol, Example 786 was obtained. ¹H NMR (500 MHz, DMSO-d₆) δ ppm: 12.71 (br s, 1H), 8.14 (d, 1H), 7.07 (d, 1H), 7.04 (t, 1H), 6.88 (d, 1H), 6.77 (d, 1H), 6.70 (dd, 1H), 6.59 (t, 1H), 6.54 (dd, 1H), 6.52 (dd, 1H), 6.23 (br s, 1H), 4.51 (m, 1H), 3.90/3.85 (dd+dd, 2H), 3.05 (m, 1H), 2.91/2.43 (dd+dd, 2H), 2.76/2.65 (dd+m, 2H), 2.44-1.30 (m, 8H), 2.11 (m, 1H), 1.98 (m, 1H), 1.80/1.74 (m+m, 2H), 1.67/1.60 (m+m, 2H), 1.50/1.33 (dd+dd, 2H), 1.26/1.25 (d+d, 6H), 1.06 (d, 3H), 1.03 (d, 3H). HRMS calculated for C₃₈H₄₇N₂O₄Cl: 630.3224; found: 631.3284 (M+H).

### Example 787 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-propoxy-2′,3′-dihydrospiro[cyclohexane-1,1′-indene]-4-carboxylic acid

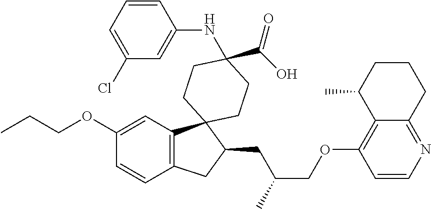 [SMILES: CCCOc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 14a as the appropriate indane and nPrOH as the appropriate alcohol, Example 787 was obtained. ¹H NMR (500 MHz, DMSO-d₆) δ ppm: 12.73 (br s, 1H), 8.14 (d, 1H), 7.07 (d, 1H), 7.02 (t, 1H), 6.89 (d, 1H), 6.76 (d, 1H), 6.70 (dd, 1H), 6.61 (t, 1H), 6.53 (dm, 1H), 6.52 (dm, 1H), 6.24 (br s, 1H), 3.89/3.85 (dd+dd, 2H), 3.87 (t, 2H), 3.05 (m, 1H), 2.91/2.44 (dd+dd, 2H), 2.76/2.65 (m+m, 2H), 2.49-1.29 (m, 12H), 2.13 (m, 1H), 1.98 (m, 1H), 1.71 (m, 2H), 1.47/1.32 (m+m, 2H), 1.05 (d, 3H), 1.04 (d, 3H), 0.97 (t, 3H). HRMS calculated for C₃₈H₄₇N₂O₄Cl: 630.3224; found: 631.3301 (M+H).

### Example 788 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-(2-methylpropoxy)-2′,3′-dihydrospiro[cyclohexane-1,1′-indene]-4-carboxylic acid

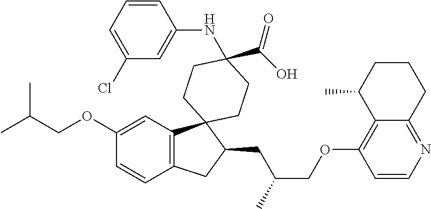 [SMILES: CC(C)COc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 14a as the appropriate indane and 2-methylpropan-1-ol as the appropriate alcohol, Example 788 was obtained. ¹H NMR (500 MHz, DMSO-d₆) δ ppm: 12.71 (br s, 1H), 8.14 (d, 1H), 7.08 (d, 1H), 7.04 (t, 1H), 6.89 (d, 1H), 6.77 (d, 1H), 6.71 (dd, 1H), 6.60 (t, 1H), 6.54 (dd, 1H), 6.52 (dd, 1H), 6.24 (br s, 1H), 3.90/3.84 (dd+dd, 2H), 3.69 (d, 2H), 3.05 (m, 1H), 2.92/2.44 (dd+dd, 2H), 2.76/2.65 (m+m, 2H), 2.44-1.34 (m, 8H), 2.14 (m, 1H), 2.00 (m, 1H), 1.98 (m, 1H), 1.80/1.73 (m+m, 2H), 1.67/1.60 (m+m, 2H), 1.48/1.33 (dd+dd, 2H), 1.05 (d, 3H), 1.04 (d, 3H), 0.97 (d, 6H). HRMS calculated for C₃₉H₄₉N₂O₄Cl: 644.3381; found: 645.3441 (M+H).

### Example 789 (1r,2′S,4S)-4-(3-chloroanilino)-6′-(cyclopentyloxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

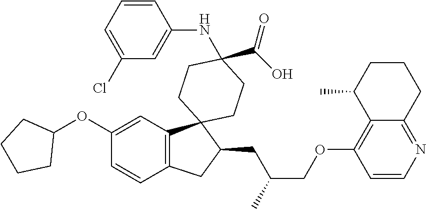 [SMILES: C[C@@H](COc1ccnc2c1[C@H](C)CCC2)C[C@H]1Cc2ccc(OC3CCCC3)cc2[C@]12CC[C@@](Nc1cccc(Cl)c1)(C(=O)O)CC2]

Using General procedure 32 and Preparation 14a as the appropriate indane and cyclopentanol as the appropriate alcohol, Example 789 was obtained. ¹H NMR (500 MHz, DMSO-d₆) δ ppm: 12.70 (br s, 1H), 8.14 (d, 1H), 7.07 (d, 1H), 7.04 (t, 1H), 6.89 (d, 1H), 6.77 (d, 1H), 6.68 (dd, 1H), 6.60 (t, 1H), 6.54 (dd, 1H), 6.52 (dd, 1H), 6.24 (br s, 1H), 4.73 (m, 1H), 3.91/3.85 (dd+dd, 2H), 3.06 (m, 1H), 2.91/2.43 (dd+dd, 2H), 2.76/2.65 (dd+m, 2H), 2.45-1.29 (m, 16H), 2.11 (m, 1H), 1.99 (m, 1H), 1.80/1.74 (m+m, 2H), 1.67/1.61 (m+m, 2H), 1.50/1.33 (m+m, 2H), 1.06 (d, 3H), 1.03 (d, 3H). HRMS calculated for C₄₀H₄₉N₂O₄Cl: 656.3381; found: 657.3439 (M+H).

### Example 790 (1r,2′R,4R)-4-(3-chloroanilino)-6′-(cyclopropylmethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

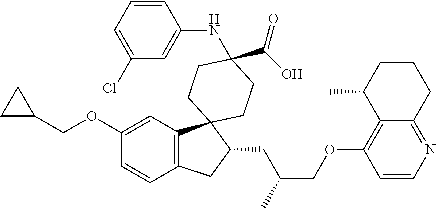 [SMILES: C[C@@H](COc1ccnc2c1[C@H](C)CCC2)C[C@@H]1Cc2ccc(OCC3CC3)cc2[C@]12CC[C@@](Nc1cccc(Cl)c1)(C(=O)O)CC2]

Using General procedure 30a and Preparation 14c as the appropriate indane and cyclopropanemethanol as the appropriate alcohol, an intermediate was obtained, which was hydrolyzed as described in General procedure 33b to obtain Example 790, as a white powder. LRMS calculated for C₃₉H₄₇ClN₂O₄: 642; found: 643 (M+H). ¹H NMR (400 MHz, DMSO-d₆) δ ppm: 12.76 (br s, 1H), 8.14 (d, J=5.6 Hz, 1H), 7.11-7.02 (m, 2H), 6.93 (d, J=2.3 Hz, 1H), 6.78 (d, J=5.6 Hz, 1H), 6.70 (dd, J=8.2, 2.3 Hz, 1H), 6.60 (t, J=2.1 Hz, 1H), 6.57-6.53 (m, 1H), 6.52-6.47 (m, 1H), 6.15 (br s, 1H), 4.01 (dd, J=9.6, 4.2 Hz, 1H), 3.87 (dd, J=9.6, 5.4 Hz, 1H), 3.76 (d, J=6.9 Hz, 2H), 3.08-2.97 (m, 1H), 2.92 (dd, J=15.2, 7.1 Hz, 1H), 2.79-2.69 (m, 1H), 2.66-2.45 (m, 2H), 2.45-2.33 (m, 1H), 2.15-1.94 (m, 4H), 1.91-1.60 (m, 6H), 1.59-1.16 (m, 6H), 1.14-1.06 (m, 6H), 0.61-0.53 (m, 2H), 0.34-0.28 (m, 2H).

### Example 791 (1r,2′S,4S)-4-(3-chloroanilino)-6′-(cyclopropylmethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

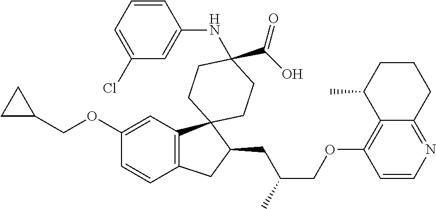 [SMILES: C[C@@H](COc1ccnc2c1[C@H](C)CCC2)C[C@H]1Cc2ccc(OCC3CC3)cc2[C@]12CC[C@@](Nc1cccc(Cl)c1)(C(=O)O)CC2]

Using General procedure 32 and Preparation 14a as the appropriate indane and cyclopropanemethanol as the appropriate alcohol, Example 791 was obtained as a white solid. LRMS calculated for C₃₉H₄₇ClN₂O₄: 642; found: 643 (M+H). ¹H NMR (400 MHz, DMSO-d₆) δ ppm: 8.15 (d, J=5.6 Hz, 1H), 7.08 (d, J=8.1 Hz, 1H), 7.03 (t, J=8.1 Hz, 1H), 6.91 (d, J=2.3 Hz, 1H), 6.77 (d, J=5.6 Hz, 1H), 6.70 (dd, J=8.1, 2.3 Hz, 1H), 6.62 (t, J=2.1 Hz, 1H), 6.58-6.47 (m, 2H), 3.91 (dd, J=9.4, 6.2 Hz, 1H), 3.85 (dd, J=9.4, 5.6 Hz, 1H), 3.76 (d, J=6.9 Hz, 2H), 3.11-3.00 (m, 1H), 2.92 (dd, J=15.2, 7.0 Hz, 1H), 2.82-2.72 (m, 1H), 2.72-2.59 (m, 1H), 2.49-2.37 (m, 2H), 2.20-2.07 (m, 2H), 2.05-1.92 (m, 2H), 1.91-1.56 (m, 7H), 1.55-1.16 (m, 5H), 1.11-0.98 (m, 6H), 0.61-0.52 (m, 2H), 0.35-0.27 (m, 2H).

### Example 792 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-(2,2,2-trifluoroethoxy)-2′,3′-dihydrospiro[cyclohexane-1,1′-indene]-4-carboxylic acid

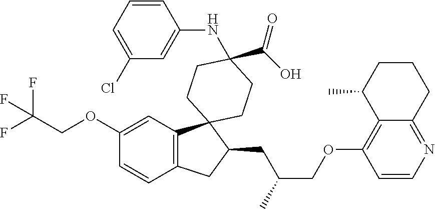 [SMILES: C[C@@H](COc1ccnc2c1[C@H](C)CCC2)C[C@H]1Cc2ccc(OCC(F)(F)F)cc2[C@]12CC[C@@](Nc1cccc(Cl)c1)(C(=O)O)CC2]

Using General procedure 32 and Preparation 14a as the appropriate indane and 2,2,2-trifluoroethanol as the appropriate alcohol, Example 792 was obtained. ¹H NMR (500 MHz, DMSO-d₆) δ ppm: 12.7 (br s, 1H), 8.14 (d, 1H), 7.14 (d, 1H), 7.03 (t, 1H), 6.96 (d, 1H), 6.85 (dd, 1H), 6.76 (d, 1H), 6.61 (t, 1H), 6.54 (dm, 1H), 6.53 (dm, 1H), 6.24 (br s, 1H), 4.71/4.68 (m+m, 2H), 3.90/3.84 (dd+dd, 2H), 3.04 (m, 1H), 2.95/2.46 (dd+dd, 2H), 2.76/2.65 (m+m, 2H), 2.49-1.32 (m, 12H), 2.17 (m, 1H), 1.98 (m, 1H), 1.46/1.32 (m+m, 2H), 1.04 (d, 3H), 1.04 (d, 3H). HRMS calculated for C₃₇H₄₂N₂O₄F₃Cl: 670.2785; found: 671.2861 (M+H).

### Example 793 (1r,2′S,4S)-4-(3-chloroanilino)-2′-[(2R)-2-methyl-3-{[(5S)-5-methyl-5,6,7,8-tetrahydroquinolin-4-yl]oxy}propyl]-6′-phenoxy-2′,3′-dihydrospiro[cyclohexane-1,1′-indene]-4-carboxylic acid

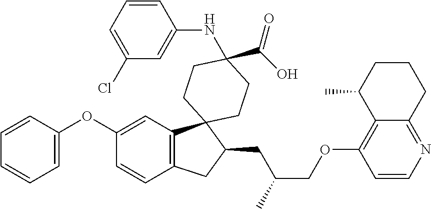 [SMILES: C[C@@H](COc1ccnc2c1[C@H](C)CCC2)C[C@H]1Cc2ccc(Oc3ccccc3)cc2[C@]12CC[C@@](Nc1cccc(Cl)c1)(C(=O)O)CC2]

Preparation 14b (345 mg, 0.493 mmol) and phenylboronic acid (1.48 mmol, 180 mg) were dissolved in dry DCM (4.9 mL). then Cu(OAc)₂ (0.99 mmol, 179 mg) dry pyridine (1.48 mmol, 0.12 mL) were added and the mixture was stirred under N₂ at rt for 1 day. Then phenylboronic acid (1.48 mmol, 180 mg) was added and further stirred for 2 days, then at 40° C. for 1 day. Then it was diluted with water and aq. NaHCO₃ solution, extracted with DCM. pH was adjusted to 6 with 1 M aq. HCl solution and extracted again with DCM. The combined organic layer was washed with brine, dried over Na₂SO₄, filtered and the filtrate was concentrated. Then it was purified via flash chromatography using hexane and EtOAc as eluents to obtain an intermediate which was hydrolyzed according to General procedure 33a to obtain Example 793 (131 mg, 0.197 mmol, 40%). ¹H NMR (500 MHz, DMSO-d₆) δ ppm: 12.66 (br s, 1H), 8.14 (d, 1H), 7.37 (t, 2H), 7.20 (d, 1H), 7.10 (t, 1H), 7.03 (t, 1H), 7.01 (d, 1H), 6.97 (d, 2H), 6.78 (dd, 1H), 6.76 (d, 1H), 6.60 (t, 1H), 6.53 (dd, 1H), 6.50 (dd, 1H), 6.22 (br s, 1H), 3.93/3.83 (dd+dd, 2H), 3.01 (m, 1H), 2.99/2.54 (dd+dd, 2H), 2.76/2.65 (m+m, 2H), 2.35-1.37 (m, 8H), 2.20 (dd, 1H), 2.00 (m, 1H), 1.80/1.73 (m+m, 2H), 1.62 (m, 2H), 1.46/1.38 (dd+dd, 2H), 1.14 (d, 3H), 1.06 (d, 3H). HRMS calculated for C₄₁H₄₅N₂O₄Cl: 664.3068; found: 665.3128 (M+H).

### Example 794 (1r,2′S,4S)-4-(3-chloroanilino)-6′-(2-methoxyethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

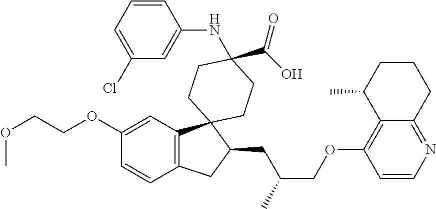 [SMILES: COCCOc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 14a as the appropriate indane and 2-methoxyethanol as the appropriate alcohol, Example 794 was obtained. ¹H NMR (500 MHz, DMSO-d₆) δ ppm: 12.64 (br s, 1H), 8.15 (d, 1H), 7.10 (d, 1H), 7.05 (t, 1H), 6.91 (d, 1H), 6.78 (d, 1H), 6.74 (dd, 1H), 6.61 (t, 1H), 6.55 (dm, 1H), 6.53 (dm, 1H), 6.25 (br s, 1H), 4.05 (m, 2H), 3.91/3.86 (dd+dd, 2H), 3.66 (m, 2H), 3.32 (s, 3H), 3.06 (m, 1H), 2.93/2.45 (dd+dd, 2H), 2.77/2.66 (dm+m, 2H), 2.48-1.30 (m, 8H), 2.13 (m, 1H), 1.99 (m, 1H), 1.86-1.67 (m, 2H), 1.68/1.63 (m+m, 2H), 1.49/1.34 (m+m, 2H), 1.06 (d, 3H), 1.04 (d, 3H). HRMS calculated for C₃₈H₄₇N₂O₅Cl: 646.3173; found: 647.3262 (M+H).

### Example 797 (1r,2′S,4S)-4-(3-chloroanilino)-2′-[(2S)-3-{[(5R)-5-methyl-5,6,7,8-tetrahydroquinolin-4-yl]oxy}-2-phenylpropyl]-2′,3′-dihydrospiro[cyclohexane-1,1′-indene]-4-carboxylic acid

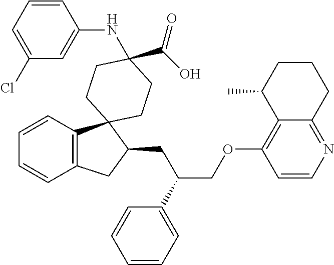 [SMILES: C[C@@H]1CCCc2nccc(OC[C@@H](C[C@H]3Cc4ccccc4[C@]34CC[C@@](Nc3cccc(Cl)c3)(C(=O)O)CC4)c3ccccc3)c21]

Using General procedure 32 and Example 547E as the appropriate indane and Preparation 2a1 as the appropriate alcohol, Example 797 was obtained. ¹H NMR (500 MHz, DMSO-d₆) δ ppm: 12.68 (br s, 1H), 8.59 (d, 1H), 7.47 (d, 1H), 7.42-7.10 (m, 9H), 7.06 (t, 1H), 6.63 (t, 1H), 6.55 (dd, 1H), 6.53 (dd, 1H), 6.21 (br s, 1H), 4.58/4.43 (dd+dd, 2H), 3.30 (m, 1H), 2.95/2.66 (dd+dd, 2H), 2.92 (m, 1H), 2.40-1.18 (m, 8H), 2.07/1.89 (m+m, 2H), 2.01/1.77 (m+m, 2H), 1.80 (m, 2H), 1.64 (m, 3H), 1.00 (d, 3H). HRMS calculated for C₄₀H₄₃N₂O₃Cl: 634.2962; found: 635.3038 (M+H).

### Example 813

### Example 813A 2-bromo-5-methoxy-6-methyl-2,3-dihydro-1H-inden-1-one

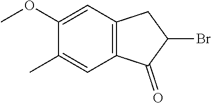

Using General procedure 5 and 5-methoxy-6-methyl-2,3-dihydro-1H-inden-1-one as the appropriate indan-1-one, Example 813A was obtained. ¹H NMR (500 MHz, DMSO-d₆) δ ppm: 7.52 (s, 1H), 7.10 (s, 1H), 4.94 (dd, 1H), 3.92 (s, 3H), 3.82 (dd, 1H), 3.25 (dd, 1H), 2.19 (s, 3H).

### Example 813B 2-bromo-5-methoxy-6-methyl-2,3-dihydro-1H-inden-1-ol

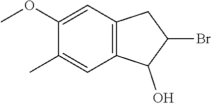

Using General procedure 6 and Example 813A as the appropriate bromo-indan-1-one, Example 813B was obtained as a single diastereoisomer. ¹H NMR (500 MHz, DMSO-d₆) δ ppm: 7.07 (s, 1H), 6.83 (s, 1H), 5.53 (d, 1H), 4.85-4.77 (m, 2H), 3.76 (s, 3H), 3.34 (dd, 1H), 3.15 (dd, 1H), 2.13 (s, 3H).

### Example 813C 2-bromo-6-methoxy-5-methyl-1H-indene

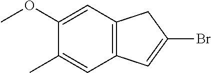

Using General procedure 7 and Example 813B as the appropriate indane, Example 813C was obtained. ¹H NMR (500 MHz, DMSO-d₆) δ ppm: 7.12 (s, 1H), 7.07 (s, 1H), 6.97 (m, 1H), 3.78 (s, 3H), 3.62 (m, 2H), 2.15 (s, 3H). HRMS calculated for C₁₁H₁₁OBr: 237.9953; found: 238.0003 (M).

### Example 813D 2″-bromo-6″-methoxy-5″-methyldispiro[[1,3]dioxolane-2,1′-cyclohexane-4′,1″-indene]

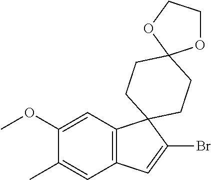 [SMILES: COc1cc2c(cc1C)C=C(Br)C21CCC2(CC1)OCCO2]

Using General procedure 8a and Example 813C as the appropriate indene, a mixture of regioisomers was obtained. The regioisomers were separated via flash chromatography using hexane and EtOAc as eluents. The regioisomer eluting later was collected as Example 813D. ¹H NMR (500 MHz, DMSO-d₆) δ ppm: 7.14 (s, 1H), 7.13 (s, 1H), 6.90 (s, 1H), 3.99-3.91 (m, 4H), 3.83 (s, 3H), 2.15 (s, 3H), 2.12/1.90 (m+m, 4H), 2.07/1.23 (m+m, 4H). HRMS calculated for C₁₈H₂₁O₃Br: 364.0674; found: 365.0748 (M+H).

### Example 813E 2′-bromo-6′-methoxy-5′-methylspiro[cyclohexane-1,1′-inden]-4-one

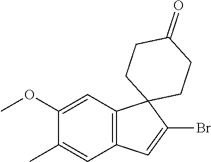 [SMILES: COc1cc2c(cc1C)C=C(Br)C21CCC(=O)CC1]

Using General procedure 9 and Example 813D as the appropriate ketal, Example 813E was obtained. ¹H NMR (500 MHz, DMSO-d₆) δ ppm: 7.38 (s, 1H), 7.15 (s, 1H), 6.96 (s, 1H), 3.85 (s, 3H), 2.95/2.52 (m+m, 4H), 2.17/1.65 (m+m, 4H), 2.16 (s, 3H).

### Example 813F 2″-bromo-6″-methoxy-5″-methyldispiro[imidazolidine-4,1′-cyclohexane-4′,1″-indene]-2,5-dione

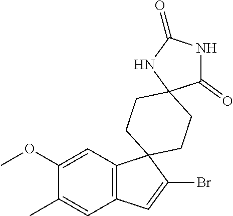 [SMILES: COc1cc2c(cc1C)C=C(Br)C21CCC2(CC1)NC(=O)NC2=O]

Using General procedure 14 and Example 813E as the appropriate ketone, Example 813F was obtained as a mixture of diastereoisomers. ¹H NMR (500 MHz, DMSO-d₆) δ ppm: 10.80/10.68 (s, 1H), 8.97/8.84 (s, 1H), 7.41/7.38 (s, 1H), 7.14/7.07 (s, 1H), 6.92/6.91 (s, 1H), 3.85 (s, 3H), 2.15/2.13 (s, 3H), 2.56-1.07 (m, 8H). HRMS calculated for C₁₈H₁₉N₂O₃Br: 390.0579; found: 391.0644 and 391.0655 (M+H).

### Example 813G 4-amino-2′-bromo-6′-methoxy-5′-methylspiro[cyclohexane-1,1′-indene]-4-carboxylic acid

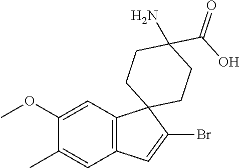 [SMILES: COc1cc2c(cc1C)C=C(Br)C21CCC(N)(C(=O)O)CC1]

Using General procedure 15 and Example 813F as the appropriate hydantoin, Example 813G was obtained as a mixture of diastereoisomers. ¹H NMR (500 MHz, DMSO-d₆) δ ppm: 7.90/7.39 (s, 1H), 7.81 (br s, 3H), 7.10/7.08 (s, 1H), 6.89/6.86 (s, 1H), 3.85 (s, 3H), 2.85-0.86 (m, 8H), 2.15/2.14 (s, 3H). HRMS calculated for C₁₇H₂₀NO₃Br: 365.0627; found: 366.0701 (M+H).

### Example 813H 2′-bromo-4-(3-chloroanilino)-6′-methoxy-5′-methylspiro[cyclohexane-1,1′-indene]-4-carboxylic acid

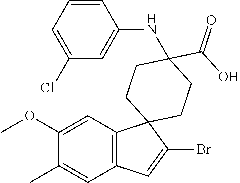 [SMILES: COc1cc2c(cc1C)C=C(Br)C21CCC(Nc2cccc(Cl)c2)(C(=O)O)CC1]

Using General procedure 16 and Example 813G as the appropriate amino acid and 1-chloro-3-iodo-benzene as the appropriate iodobenzene, Example 813H was obtained as a mixture of diastereoisomers. HRMS calculated for C₂₃H₂₃NO₃BrCl: 475.0550; found: 476.0626 (M+H).

### Example 8131 methyl (1s,4s)-2′-bromo-4-(3-chloroanilino)-6′-methoxy-5′-methylspiro[cyclohexane-1,1′-indene]-4-carboxylate

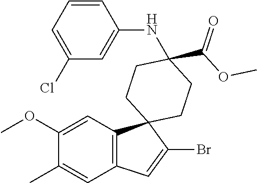

Using General procedure 17b and Example 813H as the appropriate amino acid, a mixture of diastereoisomers was obtained. The diastereoisomers were separated by chiral chromatography. Column: AD, 50×500 mm, 20 μm, Eluents: EtOH/heptane. The diastereoisomer eluting earlier was further purified by flash chromatography using heptane and EtOAC as eluents, then by prep RP-HPLC using 25 mM aq. NH₄HCO₃ solution and MeCN as eluents to obtain Example 8131. ¹H NMR (500 MHz, DMSO-d₆) δ ppm: 7.31 (s, 1H), 7.14 (s, 1H), 7.09 (t, 1H), 6.90 (s, 1H), 6.60 (dm, 1H), 6.59 (m, 1H), 6.49 (s, 1H), 6.46 (dm, 1H), 3.85 (s, 3H), 3.70 (s, 3H), 2.56-2.45 (m, 2H), 2.25 (d, 2H), 2.17 (m, 2H), 2.16 (s, 3H), 0.99 (d, 2H). HRMS calculated for C₂₄H₂₅NO₃ClBr: 489.0706; found: 490.0780 (M+H).

### Example 813J methyl (1r,4R)-4-(3-chloroanilino)-6′-methoxy-2′-{(2R)-3-[(4-methoxyphenyl)methoxy]-2-methylpropyl}-5′-methylspiro[cyclohexane-1,1′-indene]-4-carboxylate

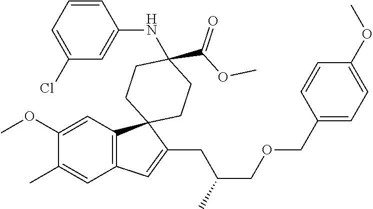 [SMILES: COc1ccc(COC[C@H](C)CC2=Cc3cc(C)c(OC)cc3[C@]23CC[C@@](Nc2cccc(Cl)c2)(C(=O)OC)CC3)cc1]

Using General procedure 27b and Example 8131 as the appropriate 2-bromo-indene derivative and Preparation 3d as the appropriate Zn reagent, Example 813J was obtained. ¹H NMR (500 MHz, DMSO-d₆) δ ppm: 7.28 (s, 1H), 7.21 (d, 2H), 7.09 (t, 1H), 7.03 (s, 1H), 6.86 (d, 2H), 6.61 (t, 1H), 6.59 (dd, 1H), 6.46 (dd, 1H), 6.42 (s, 1H), 6.30 (s, 1H), 4.39/4.36 (d+d, 2H), 3.82 (s, 3H), 3.72 (s, 3H), 3.69 (s, 3H), 3.33/3.27 (dd+dd, 2H), 2.52-0.83 (m, 8H), 2.30/1.93 (dd+dd, 2H), 2.14 (s, 3H), 2.13 (m, 1H), 0.93 (d, 3H). HRMS calculated for C₃₆H₄₂NO₅Cl: 603.2751; found: 604.2824 (M+H).

### Example 813K methyl (1r,4R)-4-(3-chloroanilino)-6′-methoxy-2′-{(2R)-3-[(4-methoxyphenyl)methoxy]-2-methylpropyl}-5′-methyl-2′,3′-dihydrospiro[cyclohexane-1,1′-indene]-4-carboxylate

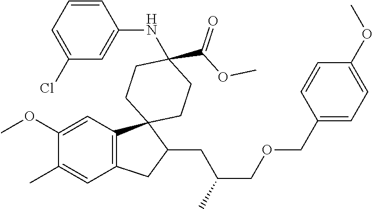

Using General procedure 19 and Example 813J as the appropriate indene, Example 813K was obtain as a mixture of diastereoisomers. ¹H NMR (500 MHz, DMSO-d₆) δ ppm: 7.20 (m, 2H), 7.06 (t, 1H), 6.94/6.93 (s, 1H), 6.91/6.90 (s, 1H), 6.86/6.80 (m, 2H), 6.62-6.41 (m, 3H), 6.32/6.31 (s, 1H), 4.42-4.30 (d+d, 2H), 3.76 (s, 3H), 3.72/3.68 (s, 3H), 3.65 (s, 3H), 3.36-3.16 (m, 2H), 2.82/2.38 (dd+dd, 2H), 2.56-0.96 (m, 11H), 2.09 (s, 3H), 2.04 (m, 1H), 0.93/0.88 (d, 3H). HRMS calculated for C₃₆H₄₄NO₅Cl: 605.2908; found: 606.2955 (M+H).

### Example 813L methyl (1r,2′S,4S)-4-(3-chloroanilino)-2′-[(2R)-3-hydroxy-2-methylpropyl]-6′-methoxy-5′-methyl-2′,3′-dihydrospiro[cyclohexane-1,1′-indene]-4-carboxylate

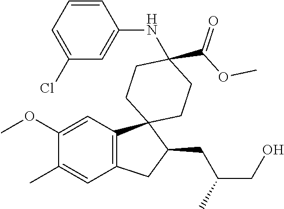 [SMILES: COc1cc2c(cc1C)C[C@H](C[C@@H](C)CO)[C@]21CC[C@](Nc2cccc(Cl)c2)(C(=O)OC)CC1]

Using General procedure 28b and Example 813K as the appropriate PMB derivative, a mixture of diastereoisomers was obtained. The diastereoisomers were separated by prep RP-HPLC using 25 mM aq. NH₄HCO₃ solution and MeCN as eluents. The diastereoisomer eluting earlier was collected as Example 813L. ¹H NMR (500 MHz, DMSO-d₆) δ ppm: 7.06 (t, 1H), 6.95 (s, 1H), 6.91 (s, 1H), 6.57 (t, 1H), 6.56 (dd, 1H), 6.44 (dd, 1H), 6.30 (s, 1H), 4.44 (t, 1H), 3.77 (s, 3H), 3.65 (s, 3H), 3.21 (t, 2H), 2.83/2.38 (dd+dd, 2H), 2.23-1.30 (m, 8H), 2.09 (s, 3H), 2.06 (m, 1H), 1.54 (m, 1H), 1.33/1.04 (t+t, 2H), 0.84 (d, 3H). HRMS calculated for C₂₈H₃₆NO₄Cl: 485.2333; found: 486.2406 (M+H).

### Example 813 (1r,2′S,4S)-4-(3-chloroanilino)-6′-methoxy-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

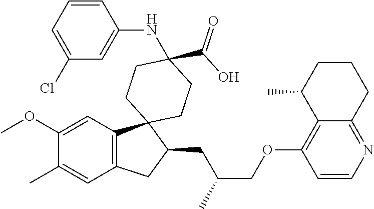 [SMILES: COc1cc2c(cc1C)C[C@H](C[C@@H](C)COc1ccnc3c1[C@H](C)CCC3)[C@]21CC[C@](Nc2cccc(Cl)c2)(C(=O)O)CC1]

Using General procedure 32 and Example 813L as the appropriate indane and Preparation 2a1 as the appropriate alcohol, Example 813 was obtained. ¹H NMR (500 MHz, DMSO-d₆) δ ppm: 12.69 (br s, 1H), 8.14 (d, 1H), 7.04 (t, 1H), 6.95 (s, 1H), 6.90 (s, 1H), 6.76 (d, 1H), 6.60 (t, 1H), 6.53 (dd, 1H), 6.52 (dd, 1H), 6.24 (br s, 1H), 3.90/3.84 (dd+dd, 2H), 3.75 (s, 3H), 3.04 (m, 1H), 2.89/2.41 (dd+dd, 2H), 2.76/2.65 (m+m, 2H), 2.52-1.36 (m, 8H), 2.14 (m, 1H), 2.09 (s, 3H), 1.97 (m, 1H), 1.81/1.74 (m+m, 2H), 1.67/1.6 (m+m, 2H), 1.46/1.31 (t+t, 2H), 1.04 (d, 3H), 1.04 (d, 3H). HRMS calculated for C₃₇H₄₅N₂O₄Cl: 616.3068; found: 617.3134 (M+H).

### Example 814

### Example 814A methyl (1r,2′S,4S)-5′-chloro-4-[(3-chlorophenyl)(trifluoroacetyl)amino]-6′-(methoxymethoxy)-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylate

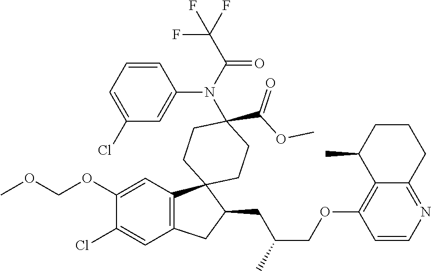 [SMILES: COCOc1cc2c(cc1Cl)C[C@H](C[C@@H](C)COc1ccnc3c1[C@@H](C)CCC3)[C@]21CC[C@@](C(=O)OC)(N(C(=O)C(F)(F)F)c2cccc(Cl)c2)CC1]

Using General procedure 30a and Preparation 15aA as the appropriate indane and Preparation 2a2 as the appropriate alcohol, Example 814A was obtained. ¹H NMR (500 MHz, DMSO-d₆) δ ppm: 8.11/8.09 (d, 1H), 7.84-7.43 (m, 4H), 7.20 (s, 1H), 6.89/6.88 (s, 1H), 6.66/6.63 (d, 1H), 5.25-5.18 (d+d, 2H), 3.81/3.62 (dd+dd, 2H), 3.79 (s, 3H), 3.40/3.39 (s, 3H), 2.97/2.50 (dd+dd, 2H), 2.80/2.74 (m, 1H), 2.73/2.61 (m+m, 2H), 2.62-0.80 (m, 14H), 2.36/2.28 (m, 1H), 1.88 (m, 1H), 1.02/0.99 (d, 3H), 0.91 (d, 3H). HRMS calculated for C₄₀H₄₅N₂O₆F₃Cl₂: 776.2607; found: 777.2692 (M+H).

### Example 814B methyl (1r,2′S,4S)-5′-chloro-4-[(3-chlorophenyl)(trifluoroacetyl)amino]-6′-hydroxy-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylate

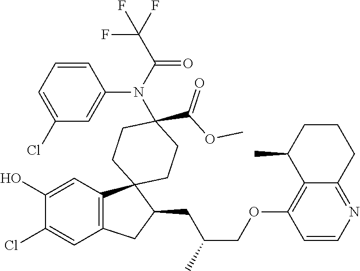 [SMILES: COC(=O)[C@]1(N(C(=O)C(F)(F)F)c2cccc(Cl)c2)CC[C@@]2(CC1)c1cc(O)c(Cl)cc1C[C@@H]2C[C@@H](C)COc1ccnc2c1[C@@H](C)CCC2]

To Example 814A (0.3 g, 0.168 mmol) 1.25 M HCl solution in EtOH (3.35 mL, 4.19 mmol) was added and the mixture was stirred at rt overnight. Then it was diluted with water and sat. aq. NaHCO₃ solution. It was extracted with DCM. The combined organic layers were dried over MgSO₄, filtered and the filtrate was concentrated under reduced pressure. The crude product was purified via flash chromatography using heptane and EtOAc as eluents to obtain Example 814B. ¹H NMR (500 MHz, DMSO-d₆) δ ppm: 9.82/9.81 (s/s, 1H), 8.10/8.09 (d/d, 1H), 7.84-7.40 (m, 4H), 7.06 (s, 1H), 6.67/6.64 (d/d, 1H), 6.64/6.62 (s/s, 1H), 3.83/3.80/3.65/3.62 (dd/dd+dd/dd, 2H), 2.91/2.43 (m+dd, 2H), 2.81/2.75 (m/m, 1H), 2.34-0.83 (m, 14H), 2.30/2.23 (m/m, 1H), 1.87 (m, 1H), 1.83-1.66 (m, 2H), 1.02/0.98 (d/d, 3H), 0.90 (d, 3H).). HRMS calculated for C₃₈H₄₁N₂O₅F₃Cl₂: 732.2345; found: 733.2411 (M+H).

### Example 814 (1r,2′S,4S)-5′-chloro-4-(3-chloroanilino)-6′-hydroxy-2′-[(2R)-2-methyl-3-{[(5S)-5-methyl-5,6,7,8-tetrahydroquinolin-4-yl]oxy}propyl]-2′,3′-dihydrospiro[cyclohexane-1,1′-indene]-4-carboxylic acid

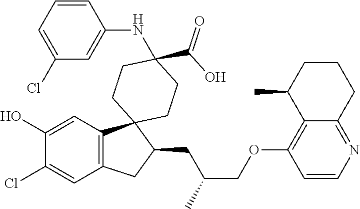 [SMILES: C[C@@H](COc1ccnc2c1[C@@H](C)CCC2)C[C@H]1Cc2cc(Cl)c(O)cc2[C@]12CC[C@@](Nc1cccc(Cl)c1)(C(=O)O)CC2]

Using General procedure 33a and Example 814B as the appropriate ester, Example 814 was obtained. ¹H NMR (500 MHz, DMSO-d₆) δ ppm: 12.70 (br s, 1H), 9.83 (s, 1H), 8.13 (d, 1H), 7.11 (s, 1H), 7.05 (t, 1H), 7.01 (s, 1H), 6.75 (d, 1H), 6.63 (m, 1H), 6.54 (m, 2H), 6.20 (br s, 1H), 3.91/3.80 (dd+dd, 2H), 2.99 (m, 1H), 2.90/2.44 (dd+dd, 2H), 2.76/2.64 (dm+m, 2H), 2.40-1.35 (m, 8H), 2.14 (m, 1H), 1.96 (m, 1H), 1.81/1.73 (m+m, 2H), 1.62 (m, 2H), 1.40/1.31 (m+m, 2H), 1.13 (d, 3H), 1.04 (d, 3H). HRMS calculated for C₃₅H₄₀N₂O₄Cl₂: 622.2365; found: 623.2434 (M+H).

### Example 815 (1r,2′S,4S)-5′-chloro-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

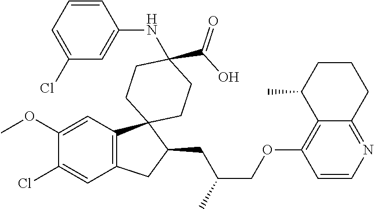 [SMILES: COc1cc2c(cc1Cl)C[C@H](C[C@@H](C)COc1ccnc3c1[C@H](C)CCC3)[C@]21CC[C@](Nc2cccc(Cl)c2)(C(=O)O)CC1]

Using General procedure 32 and Preparation 15a as the appropriate indane and MeOH as the appropriate alcohol, Example 815 was obtained. ¹H NMR (500 MHz, DMSO-d₆) δ ppm: 12.71 (br s, 1H), 8.14 (d, 1H), 7.24 (s, 1H), 7.05 (t, 1H), 7.04 (s, 1H), 6.76 (d, 1H), 6.61 (t, 1H), 6.54 (dm, 1H), 6.53 (dm, 1H), 6.28 (br s, 1H), 3.87/3.85 (dd+dd, 2H), 3.83 (s, 3H), 3.03 (m, 1H), 2.95/2.47 (dd+dd, 2H), 2.76/2.65 (m+m, 2H), 2.52-1.40 (m, 12H), 2.21 (m, 1H), 1.96 (m, 1H), 1.45/1.30 (m+m, 2H), 1.04 (d, 3H), 1.01 (d, 3H). HRMS calculated for C₃₆H₄₂N₂O₄Cl₂: 636.2521; found: 637.2595 (M+H).

### Example 816 (1r,2′S,4S)-5′-chloro-4-(3-chloroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

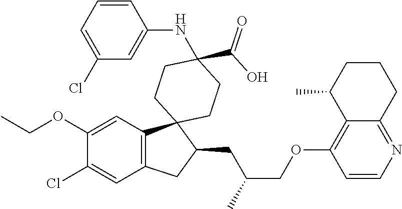 [SMILES: CCOc1cc2c(cc1Cl)C[C@H](C[C@@H](C)COc1ccnc3c1[C@H](C)CCC3)[C@]21CC[C@](Nc2cccc(Cl)c2)(C(=O)O)CC1]

Using General procedure 32 and Preparation 15a as the appropriate indane and EtOH as the appropriate alcohol, Example 816 was obtained. ¹H NMR (500 MHz, DMSO-d₆) δ ppm: 12.71 (br s, 1H), 8.14 (d, 1H), 7.24 (s, 1H), 7.05 (s, 1H), 7.04 (t, 1H), 6.76 (d, 1H), 6.60 (t, 1H), 6.54 (dd, 1H), 6.53 (dd, 1H), 6.24 (br s, 1H), 4.08 (q, 2H), 3.88/3.85 (dd+dd, 2H), 3.03 (m, 1H), 2.95/2.47 (dd+dd, 2H), 2.76/2.65 (m+m, 2H), 2.48-1.39 (m, 8H), 2.19 (m, 1H), 1.96 (m, 1H), 1.80/1.73 (m+m, 2H), 1.66/1.60 (m+m, 2H), 1.46/1.30 (t+t, 2H), 1.35 (t, 3H), 1.04 (d, 3H), 1.02 (d, 3H). HRMS calculated for C₃₇H₄₄N₂O₄Cl₂: 650.2678; found: 651.2757 (M+H).

### Example 817

### Example 817A methyl (1r,2′S,4S)-5′-bromo-4-[(3-chlorophenyl)(trifluoroacetyl)amino]-2′-[(2R)-3-hydroxy-2-methylpropyl]-6′-(methoxymethoxy)-2′,3′-dihydrospiro[cyclohexane-1,1′-indene]-4-carboxylate

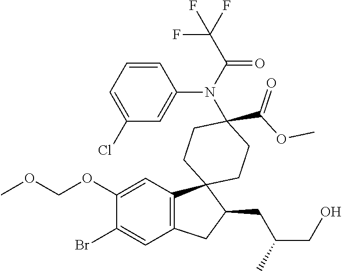 [SMILES: COCOc1cc2c(cc1Br)C[C@H](C[C@@H](C)CO)[C@]21CC[C@@](C(=O)OC)(N(C(=O)C(F)(F)F)c2cccc(Cl)c2)CC1]

Preparation 14aG (1.53 g, 2.56 mmol) was dissolved in DCM (51 mL), then PPh₃Se (87 mg, 0.26 mmol) was added and the flask was purged with N₂. NBS (546 mg, 3.07 mmol) was added portionwise, and then the mixture was stirred at rt for 90 min. The reaction mixture was diluted with water and the layers were separated. The aq. layer was extracted with DCM. The combined organic layers were dried over MgSO₄, filtered and the filtrate was concentrated under reduced pressure. The crude product was purified via flash chromatography using heptane and EtOAc as eluents to obtain Example 817A (1.19 g, 1.75 mmol, 69%). ¹H NMR (500 MHz, DMSO-d₆) δ ppm: 7.74-7.42 (m, 4H), 7.36 (s, 1H), 6.88/6.86 (s, 1H), 5.29-5.19 (d+d, 2H), 4.38/4.35 (t, 1H), 3.79/3.78 (s, 3H), 3.42/3.41 (s, 3H), 3.20-2.96 (m, 2H), 2.90/2.42 (dd+dd, 2H), 2.48-0.52 (m, 11H), 2.23/2.17 (m, 1H), 0.73/0.70 (d, 3H). HRMS calculated for C₃₀H₃₄BrClF₃NO₆: 675.121; found 693.1531 (M+NH₄).

### Example 817B methyl (1r,2′S,4S)-5′-bromo-4-[(3-chlorophenyl)(trifluoroacetyl)amino]-6′-(methoxymethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

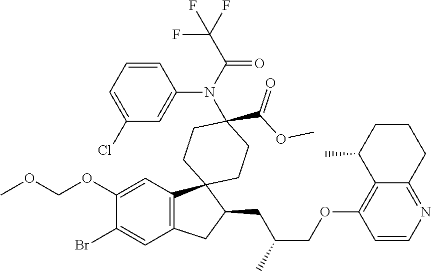 [SMILES: COCOc1cc2c(cc1Br)C[C@H](C[C@@H](C)COc1ccnc3c1[C@H](C)CCC3)[C@]21CC[C@@](C(=O)OC)(N(C(=O)C(F)(F)F)c2cccc(Cl)c2)CC1]

Using General procedure 30a and Example 817A as the appropriate indane and Preparation 2a1 as the appropriate alcohol, Example 817B was obtained. ¹H NMR (500 MHz, DMSO-d₆) δ ppm: 8.12/8.10 (d, 1H), 7.82-7.41 (m, 4H), 7.34 (s, 1H), 6.87/6.86 (s, 1H), 6.70/6.68 (d, 1H), 5.25-5.19 (d+d, 2H), 3.82-3.62 (m, 2H), 3.79 (s, 3H), 3.40/3.39 (s, 3H), 2.96/2.45 (dd+dd, 2H), 2.88 (m, 1H), 2.73/2.63 (m+m, 2H), 2.54-0.75 (m, 15H), 2.32/2.27 (m, 1H), 0.90/0.88 (d, 3H), 0.86/0.81 (d, 3H). LRMS calculated for C₄₀H₄₅BrClF₃N₂O₆: 820.21; found 821.2 (M+H).

### Example 817C methyl (1r,2′S,4S)-5′-bromo-4-[(3-chlorophenyl)(trifluoroacetyl)amino]-6′-hydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

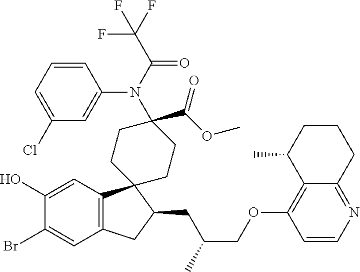 [SMILES: COC(=O)[C@]1(N(C(=O)C(F)(F)F)c2cccc(Cl)c2)CC[C@@]2(CC1)c1cc(O)c(Br)cc1C[C@@H]2C[C@@H](C)COc1ccnc2c1[C@H](C)CCC2]

Example 817B (780 mg, 0.95 mmol) was dissolved in DCM (9.5 mL). 4 M HCl solution in dioxane (1.42 mL, 5.70 mmol) was added to the mixture and stirred at rt for 8 h. Then it was diluted with water and sat. aq. NaHCO₃ solution. It was extracted with DCM. The combined organic layers were dried over MgSO₄, filtered and the filtrate was concentrated under reduced pressure. The crude product was purified via flash chromatography using heptane and EtOAc as eluents to obtain Example 817C. ¹H NMR (500 MHz, DMSO-d₆) δ ppm: 9.86/9.84 (s, 1H), 8.12/8.1 (d, 1H), 7.80-7.42 (m, 4H), 7.20 (s, 1H), 6.70/6.68 (d, 1H), 6.65/6.63 (s, 1H), 3.84-3.61 (m, 2H), 3.78 (s, 3H), 2.90/2.40 (dd+dd, 2H), 2.89 (m, 1H), 2.73/2.62 (m+m, 2H), 2.50-0.73 (m, 15H), 2.27/2.21 (m, 1H), 0.90/0.88 (d, 3H), 0.86/0.82 (d, 3H). LRMS calculated for C₃₈H₄₁BrClF₃N₂O₅: 776.18; found 777.2 (M+H).

### Example 817 (1r,2′S,4S)-5′-bromo-4-(3-chloroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

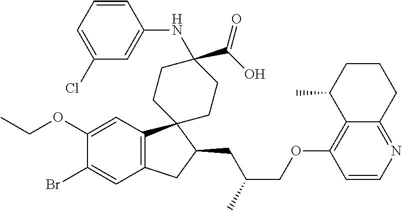 [SMILES: CCOc1cc2c(cc1Br)C[C@H](C[C@@H](C)COc1ccnc3c1[C@H](C)CCC3)[C@]21CC[C@](Nc2cccc(Cl)c2)(C(=O)O)CC1]

Using General procedure 32 and Example 817C as the appropriate indane and EtOH as the appropriate alcohol, Example 817 was obtained. ¹H NMR (500 MHz, DMSO-d₆) δ ppm: 12.64 (br s, 1H), 8.14 (d, 1H), 7.38 (s, 1H), 7.04 (t, 1H), 7.02 (s, 1H), 6.76 (d, 1H), 6.60 (t, 1H), 6.55 (dm, 1H), 6.53 (dm, 1H), 6.27 (br s, 1H), 4.09/4.06 (m+m, 2H), 3.88/3.85 (dd+dd, 2H), 3.03 (m, 1H), 2.95/2.46 (dd+dd, 2H), 2.76/2.65 (m+m, 2H), 2.51-1.35 (m, 12H), 2.19 (m, 1H), 1.96 (m, 1H), 1.46/1.30 (m+m, 2H), 1.35 (t, 3H), 1.04 (d, 3H), 1.02 (d, 3H). HRMS calculated for C₃₇H₄₄N₂O₄C₁Br: 694.2173; found: 695.2237 (M+H).

### Example 818

### Example 818A methyl (1s,4s)-2′-bromo-4-[(3-chlorophenyl)(trifluoroacetyl)amino]-5′-fluorospiro[cyclohexane-1,1′-indene]-4-carboxylate

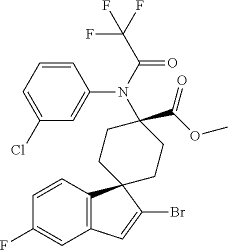 [SMILES: COC(=O)[C@]1(N(C(=O)C(F)(F)F)c2cccc(Cl)c2)CC[C@]2(CC1)C(Br)=Cc1cc(F)ccc12]

Using General procedure 22 and Example 737A as the appropriate indene, Example 818A was obtained as a yellow gum. ¹H NMR (400 MHz, DMSO-d₆) δ ppm: 7.84-7.80 (m, 1H), 7.72-7.61 (m, 3H), 7.48 (dd, J=8.4, 5.0 Hz, 1H), 7.19 (dd, J=8.9, 2.6 Hz, 1H), 7.11-7.04 (m, 1H), 7.03 (s, 1H), 3.84 (s, 3H), 2.49-2.24 (m, 4H), 1.81-1.65 (m, 1H), 1.59-1.35 (m, 3H).

### Example 818B methyl (1s,4s)-6′-acetyl-2′-bromo-4-[(3-chlorophenyl)(trifluoroacetyl)amino]-5′-fluorospiro[cyclohexane-1,1′-indene]-4-carboxylate

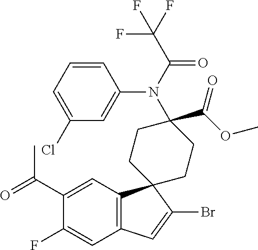 [SMILES: COC(=O)[C@]1(N(C(=O)C(F)(F)F)c2cccc(Cl)c2)CC[C@]2(CC1)C(Br)=Cc1cc(F)c(C(C)=O)cc12]

Using General procedure 23 and Example 818A as the appropriate indene, Example 818B was obtained as a yellow gum. LRMS calculated for C₂₆H₂₁BrClF₄NO₄: 601; found: 602 (M+H).

### Example 818C methyl (1s,4s)-6′-(acetyloxy)-2′-bromo-4-[(3-chlorophenyl)(trifluoroacetyl)amino]-5′-fluorospiro[cyclohexane-1,1′-indene]-4-carboxylate

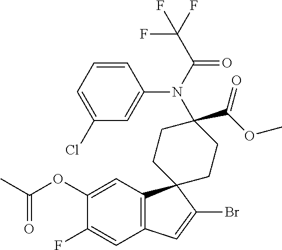 [SMILES: COC(=O)[C@]1(N(C(=O)C(F)(F)F)c2cccc(Cl)c2)CC[C@]2(CC1)C(Br)=Cc1cc(F)c(OC(C)=O)cc12]

Using General procedure 24 and Example 818B as the appropriate indene, Example 818C was obtained as a white foam. LRMS calculated for C₂₆H₂₁BrClF₄NO₅: 617; found: 616 (M−H).

### Example 818D methyl (1s,4s)-2′-bromo-4-[(3-chlorophenyl)(trifluoroacetyl)amino]-5′-fluoro-6′-hydroxyspiro[cyclohexane-1,1′-indene]-4-carboxylate

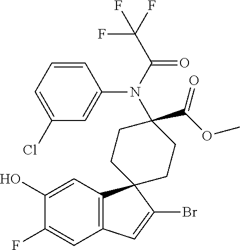 [SMILES: COC(=O)[C@]1(N(C(=O)C(F)(F)F)c2cccc(Cl)c2)CC[C@]2(CC1)C(Br)=Cc1cc(F)c(O)cc12]

Using General procedure 25 and Example 818C as the appropriate indene, Example 818D was obtained as a white solid. LRMS calculated for C₂₄H₁₉BrClF₄NO₄: 575; found: 574 (M−H). ¹H NMR (400 MHz, DMSO-d₆) δ ppm: 9.93 (s, 1H), 7.80-7.77 (m, 1H), 7.71-7.59 (m, 3H), 7.14-7.07 (m, 2H), 6.86 (s, 1H), 3.85 (s, 3H), 2.43-2.35 (m, 2H), 2.31-2.23 (m, 2H), 1.74-1.44 (m, 4H).

### Example 818E methyl (1s,4s)-2′-bromo-4-[(3-chlorophenyl)(trifluoroacetyl)amino]-5′-fluoro-6′-(methoxymethoxy)spiro[cyclohexane-1,1′-indene]-4-carboxylate

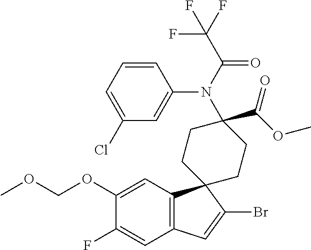

To a solution of Example 818D (937 mg, 1.62 mmol, 1 eq) in DCM (55 mL) cooled to 0° C., was added DIPEA (1.08 mL, 6.5 mmol, 4 eq) followed by MOM-Cl (0.37 mL, 4.87 mmol, 3 eq) and the mixture was stirred at rt for 18 h. The mixture was partitioned between DCM and sat. aq. NaHCO₃ solution, and the organic phase was washed with brine, dried (MgSO₄) and concentrated in vacuo. Purification by automated flash chromatography (CombiFlash Rf, 24 g RediSep™ silica cartridge) eluting with a gradient of 0-26% EtOAc in heptane afforded Example 818E as a white solid (837 mg, 1.35 mmol, 83%). ¹H NMR (400 MHz, DMSO-d₆) δ ppm: 7.84-7.80 (m, 1H), 7.72-7.59 (m, 3H), 7.41 (d, J=7.6 Hz, 1H), 7.25 (d, J=10.8 Hz, 1H), 6.95 (s, 1H), 5.25 (s, 2H), 3.84 (s, 3H), 3.47 (s, 3H), 2.55-2.21 (m, 4H), 1.81-1.67 (m, 1H), 1.60-1.27 (m, 3H).

### Example 818F methyl (1r,4R)-4-[(3-chlorophenyl)(trifluoroacetyl)amino]-5′-fluoro-6′-(methoxymethoxy)-2′-{(2R)-3-[(4-methoxyphenyl)methoxy]-2-methylpropyl}spiro[cyclohexane-1,1′-indene]-4-carboxylate

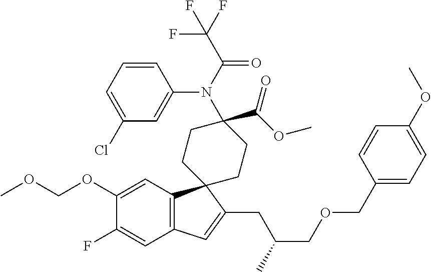 [SMILES: COCOc1cc2c(cc1F)C=C(C[C@@H](C)COCc1ccc(OC)cc1)[C@]21CC[C@@](C(=O)OC)(N(C(=O)C(F)(F)F)c2cccc(Cl)c2)CC1]

Using General procedure 27b and Example 818E as the appropriate 2-bromo-indene and Preparation 3d as the appropriate Zn reagent, Example 818F was obtained as a colourless oil. ¹H NMR (400 MHz, DMSO-d₆) δ ppm: 7.89-7.86/7.82-7.79 (m, 1H), 7.74-7.62 (m, 2H), 7.56/7.51 (t, J=8.1 Hz, 1H), 7.41 (d, J=7.7 Hz, 1H), 7.28-7.21 (m, 2H), 7.15-7.09 (m, 1H), 6.93-6.87 (m, 2H), 6.37-6.31 (m, 1H), 5.19 (s, 2H), 4.47-4.33 (m, 2H), 3.83 (s, 3H), 3.74 (s, 3H), 3.47 (s, 3H), 3.39-3.25 (m, 2H), 2.64-2.03 (m, 6H), 1.96-1.86/1.86-1.76 (m, 1H), 1.72-1.57 (m, 1H), 1.46-1.32 (m, 1H), 1.18-0.89 (m, 5H).

### Example 818G methyl (1r,4R)-4-[(3-chlorophenyl)(trifluoroacetyl)amino]-5′-fluoro-2′-[(2R)-3-hydroxy-2-methylpropyl]-6′-(methoxymethoxy)spiro[cyclohexane-1,1′-indene]-4-carboxylate

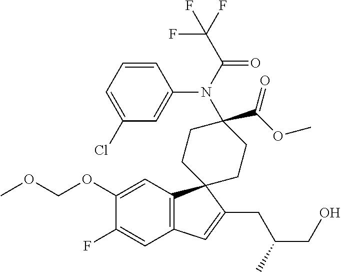 [SMILES: COCOc1cc2c(cc1F)C=C(C[C@@H](C)CO)[C@]21CC[C@@](C(=O)OC)(N(C(=O)C(F)(F)F)c2cccc(Cl)c2)CC1]

Using General procedure 28a and Example 818F as the appropriate PMB derivative, Example 818G was obtained as a cream powder. ¹H NMR (400 MHz, DMSO-d₆) δ ppm: 7.91-7.83 (m, 1H), 7.78-7.66 (m, 2H), 7.62 (t, J=8.0 Hz, 1H), 7.42 (d, J=7.6 Hz, 1H), 7.14 (d, J=11.0 Hz, 1H), 6.37-6.33 (m, 1H), 5.19 (s, 2H), 4.59-4.52 (m, 1H), 3.83 (s, 3H), 3.47 (s, 3H), 3.43-3.23 (m, 2H), 2.66-2.07 (m, 5H), 1.97-1.36 (m, 4H), 1.22-0.95 (m, 2H), 0.93-0.83 (m, 3H).

### Example 818H methyl (1r,2′S,4S)-4-[(3-chlorophenyl)(trifluoroacetyl)amino]-5′-fluoro-2′-[(2R)-3-hydroxy-2-methylpropyl]-6′-(methoxymethoxy)-2′,3′-dihydrospiro[cyclohexane-1,1′-indene]-4-carboxylate

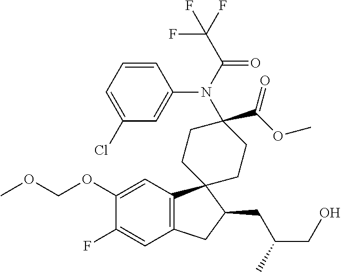 [SMILES: COCOc1cc2c(cc1F)C[C@H](C[C@@H](C)CO)[C@]21CC[C@@](C(=O)OC)(N(C(=O)C(F)(F)F)c2cccc(Cl)c2)CC1]

Using General procedure 19 and Example 818G as the appropriate indene, a mixture of diastereoisomers was obtained. They were purified and separated by flash chromatography, using heptane and EtOAc as eluent. The diastereoisomer eluting later was collected as Example 818H, isolated as a colourless gum. ¹H NMR (400 MHz, DMSO-d₆) δ ppm: 7.74-7.44 (m, 4H), 7.03 (d, J=10.9 Hz, 1H), 6.92-6.86 (m, 1H), 5.20-5.13 (m, 2H), 4.40-4.32 (m, 1H), 3.79/3.79 (s, 3H), 3.42/3.42 (s, 3H), 3.19-2.98 (m, 2H), 2.96-2.85 (m, 1H), 2.47-2.36 (m, 1H), 2.27-2.12 (m, 2H), 2.04-1.38 (m, 7H), 1.32-1.20 (m, 1H), 1.10-0.90 (m, 1H), 0.77-0.55 (m, 4H).

### Example 8181 methyl (1r,2′S,4S)-4-[(3-chlorophenyl)(trifluoroacetyl)amino]-5′-fluoro-6′-(methoxymethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

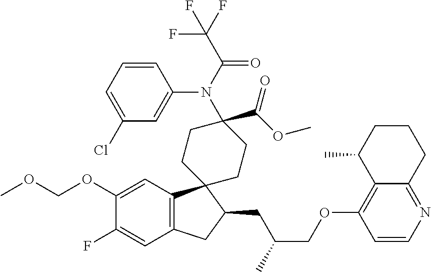 [SMILES: COCOc1cc2c(cc1F)C[C@H](C[C@@H](C)COc1ccnc3c1[C@H](C)CCC3)[C@]21CC[C@@](C(=O)OC)(N(C(=O)C(F)(F)F)c2cccc(Cl)c2)CC1]

Using General procedure 30a and Example 818H as the appropriate indane and Preparation 2a1 as the appropriate alcohol, Example 8181 was obtained as a cream powder. LRMS calculated for C₄₀H₄₅ClF₄N₂O₆: 760; found: 761 (M+H).

### Example 818J methyl (1r,2′S,4S)-4-[(3-chlorophenyl)(trifluoroacetyl)amino]-5′-fluoro-6′-hydroxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

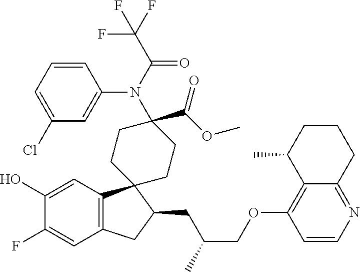 [SMILES: COC(=O)[C@]1(N(C(=O)C(F)(F)F)c2cccc(Cl)c2)CC[C@@]2(CC1)c1cc(O)c(F)cc1C[C@@H]2C[C@@H](C)COc1ccnc2c1[C@H](C)CCC2]

To a solution of Example 8181 (153 mg, 0.2 mmol, 1 eq) in DCM (3 mL) was added 4 M HCl solution in 1,4-dioxane (0.1 mL, 0.4 mmol, 2 eq) and the mixture was stirred at rt for 2 h. The mixture was partitioned between DCM and sat. aq. NaHCO₃ solution, and the organic phase was washed with brine, dried (MgSO₄) and concentrated in vacuo. Purification by automated flash chromatography (CombiFlash Rf, 4 g RediSep™ silica cartridge) eluting with a gradient of 0-20% MeOH in DCM afforded Example 818J as a white powder (115 mg, 0.16 mmol, 80%). LRMS calculated for C₃₈H₄₁ClF₄N₂O₅: 716; found: 717 (M+H).

### Example 818K methyl (1r,2′S,4S)-4-[(3-chlorophenyl)(trifluoroacetyl)amino]-6′-ethoxy-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

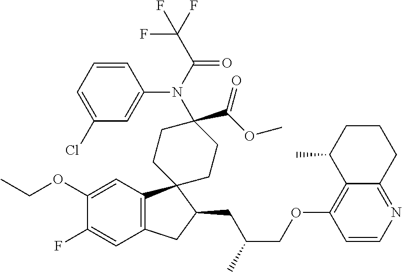 [SMILES: CCOc1cc2c(cc1F)C[C@H](C[C@@H](C)COc1ccnc3c1[C@H](C)CCC3)[C@]21CC[C@@](C(=O)OC)(N(C(=O)C(F)(F)F)c2cccc(Cl)c2)CC1]

Using General procedure 30a and Example 818J as the appropriate indane and EtOH as the appropriate alcohol, Example 818K was obtained as a white powder. LRMS calculated for C₄₀H₄₅ClF₄N₂O₅: 744; found: 745 (M+H).

### Example 818 (1r,2′S,4S)-4-(3-chloroanilino)-6′-ethoxy-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

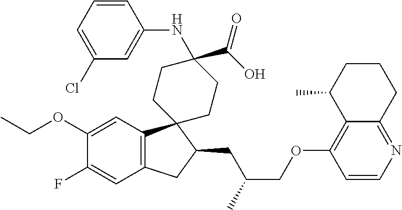 [SMILES: CCOc1cc2c(cc1F)C[C@H](C[C@@H](C)COc1ccnc3c1[C@H](C)CCC3)[C@]21CC[C@](Nc2cccc(Cl)c2)(C(=O)O)CC1]

Using General procedure 33a and Example 818K as the appropriate indane, Example 818 was obtained as a white powder. LRMS calculated for C₃₇H₄₄ClFN₂O₄: 634; found: 635 (M+H). ¹H NMR (400 MHz, DMSO-d₆) δ ppm: 8.18 (d, J=5.7 Hz, 1H), 7.10-7.01 (m, 3H), 6.82 (d, J=5.7 Hz, 1H), 6.61 (t, J=2.1 Hz, 1H), 6.57-6.51 (m, 2H), 4.14-4.02 (m, 2H), 3.96-3.84 (m, 2H), 3.10-3.00 (m, 1H), 2.95 (dd, J=15.5, 7.0 Hz, 1H), 2.83-2.73 (m, 1H), 2.73-2.62 (m, 1H), 2.51-2.40 (m, 2H), 2.23-2.13 (m, 2H), 2.05-1.92 (m, 2H), 1.91-1.57 (m, 7H), 1.54-1.27 (m, 7H), 1.09-1.00 (m, 6H).

### Example 819 (1r,2′S,4S)-5′-chloro-4-(3-chloroanilino)-2′-[(2R)-2-methyl-3-{[(5R)-5-methyl-5,6,7,8-tetrahydroquinolin-4-yl]oxy}propyl]-6′-[(propan-2-yl)oxy]-2′,3′-dihydrospiro[cyclohexane-1,1′-indene]-4-carboxylic acid

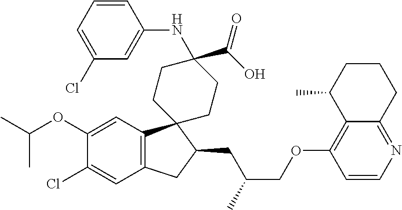 [SMILES: CC(C)Oc1cc2c(cc1Cl)C[C@H](C[C@@H](C)COc1ccnc3c1[C@H](C)CCC3)[C@]21CC[C@](Nc2cccc(Cl)c2)(C(=O)O)CC1]

Using General procedure 32 and Preparation 15a as the appropriate indane and iPrOH as the appropriate alcohol, Example 819 was obtained. ¹H NMR (400/500 MHz, DMSO-d₆) δ ppm: 12.75 (br s, 1H), 8.14 (d, 1H), 7.24 (s, 1H), 7.09 (s, 1H), 7.04 (t, 1H), 6.77 (d, 1H), 6.59 (t, 1H), 6.54 (dm, 1H), 6.52 (dm, 1H), 6.27 (br s, 1H), 4.53 (sp, 3H), 3.89/3.85 (dd+dd, 2H), 3.04 (m, 1H), 2.94/2.46 (dd+dd, 2H), 2.76/2.65 (m+m, 2H), 2.50-1.30 (m, 12H), 2.16 (m, 1H), 1.96 (m, 1H), 1.49/1.31 (m+m, 2H), 1.30 (d, 3H), 1.29 (d, 1H), 1.03 (d, 3H), 1.03 (d, 3H). HRMS calculated for C₃₈H₄₆N₂O₄Cl₂: 664.2834; found: 665.2922 (M+H).

### Example 820 (1r,2′S,4S)-5′-chloro-4-(3-chloroanilino)-2′-[(2R)-2-methyl-3-{[(5R)-5-methyl-5,6,7,8-tetrahydroquinolin-4-yl]oxy}propyl]-6′-(2-methylpropoxy)-2′,3′-dihydrospiro[cyclohexane-1,1′-indene]-4-carboxylic acid

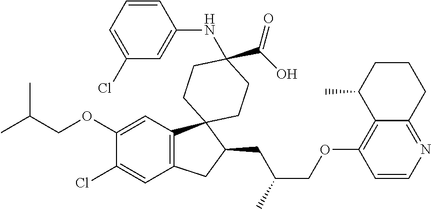 [SMILES: CC(C)COc1cc2c(cc1Cl)C[C@H](C[C@@H](C)COc1ccnc3c1[C@H](C)CCC3)[C@]21CC[C@](Nc2cccc(Cl)c2)(C(=O)O)CC1]

Using General procedure 32 and Preparation 15a as the appropriate indane and 2-methylpropan-1-ol as the appropriate alcohol, Example 820 was obtained. HRMS calculated for C₃₉H₄₈N₂O₄Cl₂: 678.2991; found: 679.3044 (M+H).

### Example 821 (1r,2′S,4S)-5′-chloro-4-(3-chloroanilino)-6′-(cyclopentyloxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

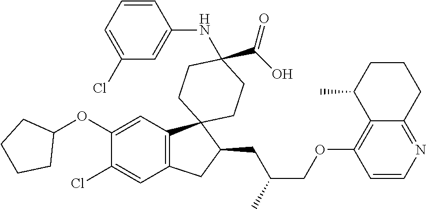 [SMILES: C[C@@H](COc1ccnc2c1[C@H](C)CCC2)C[C@H]1Cc2cc(Cl)c(OC3CCCC3)cc2[C@]12CC[C@@](Nc1cccc(Cl)c1)(C(=O)O)CC2]

Using General procedure 32 and Preparation 15a as the appropriate indane and cyclopentanol as the appropriate alcohol, Example 821 was obtained. HRMS calculated for C₄₀H₄₈N₂O₄Cl₂: 690.2991; found: 691.3051 (M+H).

### Example 822 (1r,2′S,4S)-5′-chloro-4-(3-chloroanilino)-6′-(2-methoxyethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

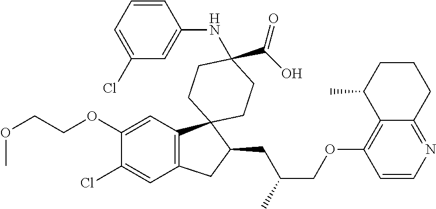 [SMILES: COCCOc1cc2c(cc1Cl)C[C@H](C[C@@H](C)COc1ccnc3c1[C@H](C)CCC3)[C@]21CC[C@](Nc2cccc(Cl)c2)(C(=O)O)CC1]

Using General procedure 32 and Preparation 15a as the appropriate indane and 2-methoxyethanol as the appropriate alcohol, Example 822 was obtained. ¹H NMR (500 MHz, DMSO-d₆) δ ppm: 12.72 (br s, 1H), 8.14 (d, 1H), 7.24 (s, 1H), 7.08 (s, 1H), 7.04 (t, 1H), 6.76 (d, 1H), 6.61 (t, 1H), 6.54 (dd, 1H), 6.53 (dd, 1H), 6.25 (br s, 1H), 4.15 (m, 2H), 3.88/3.85 (dd+dd, 2H), 3.68 (t, 2H), 3.33 (s, 3H), 3.03 (m, 1H), 2.95/2.47 (dd+dd, 2H), 2.75/2.65 (m+m, 2H), 2.48-1.26 (m, 8H), 2.19 (dd, 1H), 1.97 (m, 1H), 1.80/1.74 (m+m, 2H), 1.66/1.60 (m+m, 2H), 1.46/1.30 (t+t, 2H), 1.04 (d, 3H), 1.02 (d, 3H). HRMS calculated for C₃₈H₄₆N₂O₅Cl₂: 680.2784; found: 681.2841 (M+H).

### Example 823 (1r,2′S,4S)-5′-chloro-4-(3-chloroanilino)-6′-(3-methoxypropoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

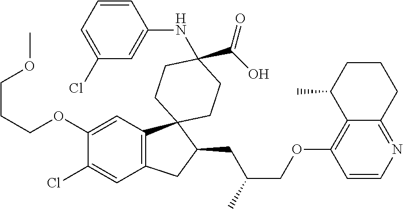 [SMILES: COCCCOc1cc2c(cc1Cl)C[C@H](C[C@@H](C)COc1ccnc3c1[C@H](C)CCC3)[C@]21CC[C@](Nc2cccc(Cl)c2)(C(=O)O)CC1]

Using General procedure 32 and Preparation 15a as the appropriate indane and 3-methoxypropan-1-ol as the appropriate alcohol, Example 823 was obtained. ¹H NMR (500 MHz, DMSO-d₆) δ ppm: 12.72 (br s, 1H), 8.14 (d, 1H), 7.24 (s, 1H), 7.05 (t, 1H), 7.04 (s, 1H), 6.76 (d, 1H), 6.61 (t, 1H), 6.54 (dm, 1H), 6.53 (dm, 1H), 6.25 (br s, 1H), 4.06/4.06 (m+m, 2H), 3.87/3.84 (dd+dd, 2H), 3.49 (t, 2H), 3.25 (s, 3H), 3.02 (m, 1H), 2.95/2.46 (dd+dd, 2H), 2.75/2.65 (m+m, 2H), 2.51-1.37 (m, 12H), 2.20 (m, 1H), 1.97 (m, 1H), 1.96 (m, 2H), 1.45/1.29 (m+m, 2H), 1.04 (d, 3H), 1.01 (d, 3H). HRMS calculated for C₃₉H₄₈N₂O₅Cl₂: 694.2940; found: 695.2990 (M+H).

### Example 824

### Example 824A tert-butyl(dimethyl)[(5-methyl-1H-inden-3-yl)oxy]silane

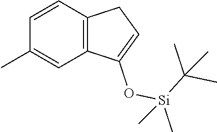 [SMILES: Cc1ccc2c(c1)C(O[Si](C)(C)C(C)(C)C)=CC2]

To NaHMDS solution in THE (1.0 M, 142 mL, 142 mmol, 1.3 eq) at 0° C. was added a solution of 6-methyl-2,3-dihydro-1H-inden-1-one (15.9 g, 109 mmol, 1 eq) in THE (360 mL) dropwise. The mixture was stirred for 10 min then a solution of TBDMS-Cl (19.7 g, 131 mmol, 1.2 eq) in THF (120 mL) was added dropwise. The mixture was stirred at 0° C. for 2 h then at rt for 16 h before quenching with sat. aq. NaHCO₃ solution. The mixture was partitioned between EtOAc and water, and the organic phase was washed with brine, dried (MgSO₄), filtered and concentrated in vacuo to afford Example 824A as a brown oil (28.4 g, 109 mmol, 100%). LRMS calculated for C₁₆H₂₄OSi: 260; found 261 (M+H).

### Example 824B tert-butyl(dimethyl)[(5″-methyldispiro[[1,3]dioxolane-2,1′-cyclohexane-4′,1″-inden]-3″-yl)oxy]silane

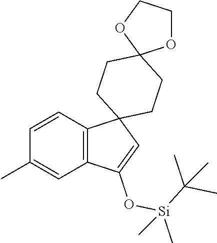 [SMILES: Cc1ccc2c(c1)C(O[Si](C)(C)C(C)(C)C)=CC21CCC2(CC1)OCCO2]

Using General procedure 8b and Example 824A as the appropriate indene, Example 824B was obtained as a brown oil (50.8 g, 109 mmol, 100%). LRMS calculated for C₂₃H₃₄O₃Si: 386; found 387 (M+H).

### Example 824C 5″-methyldispiro[[1,3]dioxolane-2,1′-cyclohexane-4′,1″-inden]-3″(2″H)-one

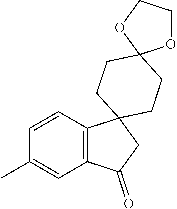 [SMILES: Cc1ccc2c(c1)C(=O)CC21CCC2(CC1)OCCO2]

Using General procedure 29 and Example 824B as the appropriate silyl intermediate, Example 824C was obtained as a a peach solid (8.96 g, 32.9 mmol, 32%). LRMS calculated for C₁₇H₂₀O₃: 272; found 273 (M+H).

### Example 824D 2″-bromo-5″-methyldispiro[[1,3]dioxolane-2,1′-cyclohexane-4′,1″-inden]-3″(2″H)-one

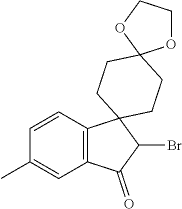 [SMILES: Cc1ccc2c(c1)C(=O)C(Br)C21CCC2(CC1)OCCO2]

To a solution of Example 824C (1 g, 3.67 mmol, 1 eq) in THE (12 mL) at 0° C. was added Br₂ (0.19 mL, 3.11 g/mL, 3.67 mmol, 1 eq) dropwise. The mixture was stirred at 0° C. for 30 min, then at rt for 1 h. DIPEA (1.22 mL, 7.34 mmol, 2 eq) was added and the mixture was stirred for 10 min before partitioning between EtOAc and water. The combined organic extracts were washed with brine, dried (MgSO₄), filtered and concentrated in vacuo. Purification by reverse phase automated flash chromatography (CombiFlash Rf, C18 50 g RediSep column) eluting with a gradient of 15-100% MeCN in water afforded Example 824D as a white solid (773 mg, 2.20 mmol, 60%). LRMS calculated for C₁₇H₁₉BrO₃: 350; found 351 (M+H).

### Example 824E 2″-bromo-5″-methyl-2″,3″-dihydrodispiro[[1,3]dioxolane-2,1′-cyclohexane-4′,1″-inden]-3″-ol

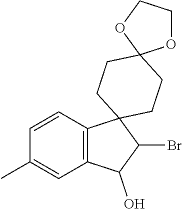 [SMILES: Cc1ccc2c(c1)C(O)C(Br)C21CCC2(CC1)OCCO2]

Using General procedure 6 and Example 824D as the appropriate ketone, Example 824E was obtained as a white solid (652 mg, 1.85 mmol, 84%). ¹H NMR (400 MHz, CDCl₃) δ ppm: 7.25-7.22 (m, 1H), 7.18-7.10 (m, 2H), 5.14 (d, J=4.6 Hz, 1H), 5.08 (d, J=4.6 Hz, 1H), 4.04-3.94 (m, 4H), 2.42-2.25 (m, 4H), 2.20-2.11 (m, 1H), 1.89-1.69 (m, 6H).

### Example 824F 2″-bromo-5″-methyldispiro[[1,3]dioxolane-2,1′-cyclohexane-4′,1″-indene]

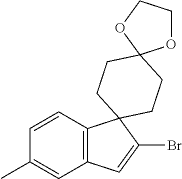 [SMILES: Cc1ccc2c(c1)C=C(Br)C21CCC2(CC1)OCCO2]

To a solution of Example 824E (650 mg, 1.84 mmol, 1 eq) in CHCl₃ (30 mL) was added PTSA (0.01 mL, 0.09 mmol, 0.05 eq) and the mixture was heated at reflux for 20 h. The mixture was cooled to rt and partitioned between DCM and sat. aq. NaHCO₃ solution. The combined organic extracts were washed with brine, dried (MgSO₄) and concentrated in vacuo. Purification by automated flash column chromatography (CombiFlash Rf, 24 g RediSep™ silica cartridge) eluting with a gradient of 0-10% EtOAc in heptane afforded Example 824F as a white solid (387 mg, 1.15 mmol, 63%). LRMS calculated for C₁₇H₁₉BrO₂: 334; found 335 (M+H).

### Example 824G 2′-bromo-5′-methylspiro[cyclohexane-1,1′-inden]-4-one

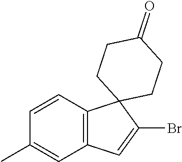 [SMILES: Cc1ccc2c(c1)C=C(Br)C21CCC(=O)CC1]

Using General procedure 9 and Example 824F as the appropriate ketone, Example 824G was obtained as a white solid (282 mg, 0.97 mmol, 67%). LRMS calculated for C₁₅H₁₅BrO: 290; found 291 (M+H).

### Example 824H 2″-bromo-5″-methyldispiro[imidazolidine-4,1′-cyclohexane-4′,1″-indene]-2,5-dione

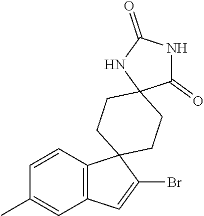 [SMILES: Cc1ccc2c(c1)C=C(Br)C21CCC2(CC1)NC(=O)NC2=O]

Using General procedure 14 and Example 824G as the appropriate ketone and NaCN instead of KCN, Example 824H was obtained as a mixture of diastereoisomers, isolated as a white solid (183 mg, 0.51 mmol, 53%). LRMS calculated for C₁₇H₁₇BrN₂O₂: 360; found 361 (M+H).

### Example 824I 4-amino-2′-bromo-5′-methylspiro[cyclohexane-1,1′-indene]-4-carboxylic acid

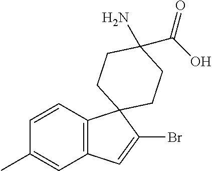 [SMILES: Cc1ccc2c(c1)C=C(Br)C21CCC(N)(C(=O)O)CC1]

Using General procedure 15 and Example 824H as the appropriate hydantoin, Example 8241 was obtained as a mixture of diastereoisomers, isolated as a white solid (1.48 g, 4.4 mmol, 92%). LRMS calculated for C₁₆H₁₈BrNO₂: 335; found 336 (M+H).

### Example 824J (1s,4s)-2′-bromo-4-(3-chloroanilino)-5′-methylspiro[cyclohexane-1,1′-indene]-4-carboxylic acid

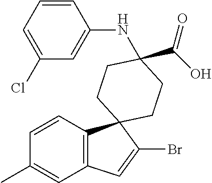 [SMILES: Cc1ccc2c(c1)C=C(Br)[C@]21CC[C@](Nc2cccc(Cl)c2)(C(=O)O)CC1]

Using General procedure 16 and Example 8241 as the appropriate amino acid and 1-chloro-3-iodobenzene as the appropriate iodobenzene, Example 824J was obtained as a yellow solid (84 mg, 0.19 mmol, 63%). LRMS calculated for C₂₂H₂₁BrClNO₂: 445; found 446 (M+H). ¹H NMR (400 MHz, CDCl₃) δ ppm: 7.66 (d, J=7.7 Hz, 1H), 7.16-7.10 (m, 2H), 7.04-6.99 (m, 1H), 6.83-6.78 (m, 2H), 6.74 (t, J=2.1 Hz, 1H), 6.64-6.59 (m, 1H), 2.70-2.57 (m, 2H), 2.38 (s, 3H), 2.35-2.26 (m, 2H), 2.19-2.07 (m, 2H), 1.21-1.13 (m, 2H).

### Example 824K methyl (1s,4s)-2′-bromo-4-(3-chloroanilino)-5′-methylspiro[cyclohexane-1,1′-indene]-4-carboxylate

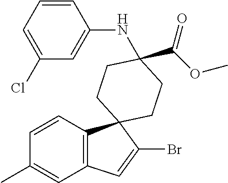

Using General procedure 17a and Example 824J as the appropriate amino acid, Example 824K was obtained as a cream solid. LRMS calculated for C₂₃H₂₃BrClNO₂: 459; found: 460 (M+H).

### Example 824L methyl (1s,4s)-2′-bromo-4-[(3-chlorophenyl)(trifluoroacetyl)amino]-5′-methylspiro[cyclohexane-1,1′-indene]-4-carboxylate

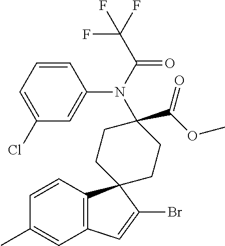

Using General procedure 22 and Example 824K as the appropriate indene, Example 824L was obtained as a yellow solid. ¹H NMR (400 MHz, DMSO-d₆) δ ppm: 7.83-7.77 (m, 1H), 7.72-7.58 (m, 3H), 7.40-7.32 (m, 1H), 7.15-7.10 (m, 1H), 7.06-6.99 (m, 1H), 6.95 (s, 1H), 3.84 (s, 3H), 2.54-2.21 (m, 7H), 1.81-1.35 (m, 4H).

### Example 824M methyl (1s,4s)-6′-acetyl-2′-bromo-4-[(3-chlorophenyl)(trifluoroacetyl)amino]-5′-methylspiro[cyclohexane-1,1′-indene]-4-carboxylate

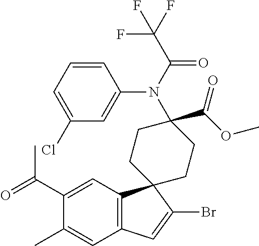 [SMILES: COC(=O)[C@]1(N(C(=O)C(F)(F)F)c2cccc(Cl)c2)CC[C@]2(CC1)C(Br)=Cc1cc(C)c(C(C)=O)cc12]

Using General procedure 23 and Example 824L as the appropriate indene, Example 824M was obtained as a white solid. LRMS calculated for C₂₇H₂₄BrClF₃NO₄: 597; found: 598 (M+H).

### Example 824N methyl (1s,4s)-6′-(acetyloxy)-2′-bromo-4-[(3-chlorophenyl)(trifluoroacetyl)amino]-5′-methylspiro[cyclohexane-1,1′-indene]-4-carboxylate

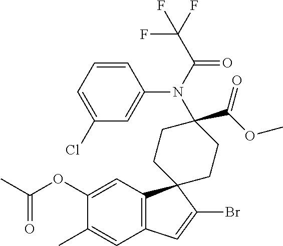 [SMILES: COC(=O)[C@]1(N(C(=O)C(F)(F)F)c2cccc(Cl)c2)CC[C@]2(CC1)C(Br)=Cc1cc(C)c(OC(C)=O)cc12]

Using General procedure 24 and Example 824M as the appropriate indene, Example 824N was obtained as an off white solid. ¹H NMR (400 MHz, DMSO-d₆) δ ppm: 7.81-7.76 (m, 1H), 7.71-7.59 (m, 3H), 7.24-7.21 (m, 1H), 7.12-7.09 (m, 1H), 6.98 (s, 1H), 3.83 (s, 3H), 2.45-2.23 (m, 7H), 2.10 (d, J=0.7 Hz, 3H), 1.78-1.63 (m, 1H), 1.61-1.38 (m, 3H).

### Example 8240 methyl (1s,4s)-2′-bromo-4-[(3-chlorophenyl)(trifluoroacetyl)amino]-6′-hydroxy-5′-methylspiro[cyclohexane-1,1′-indene]-4-carboxylate

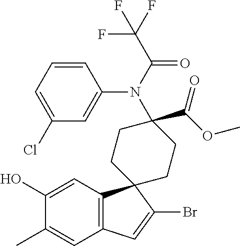

Using General procedure 25 and Example 824N as the appropriate indene, Example 8240 was obtained as an off-white solid. LRMS calculated for C₂₅H₂₂BrClF₃NO₄: 571; found: 572 (M+H).

### Example 824P methyl (1s,4s)-2′-bromo-4-[(3-chlorophenyl)(trifluoroacetyl)amino]-6′-ethoxy-5′-methylspiro[cyclohexane-1,1′-indene]-4-carboxylate

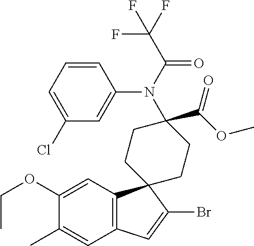 [SMILES: CCOc1cc2c(cc1C)C=C(Br)[C@]21CC[C@@](C(=O)OC)(N(C(=O)C(F)(F)F)c2cccc(Cl)c2)CC1]

Using General procedure 30a with Example 8240 as the appropriate indene and EtOH as the appropriate alcohol, Example 824P was obtained as a white solid. ¹H NMR (400 MHz, DMSO-d₆) δ ppm: 7.83-7.78 (m, 1H), 7.72-7.60 (m, 3H), 7.13-7.08 (m, 2H), 6.88 (s, 1H), 4.09 (q, J=7.0 Hz, 2H), 3.84 (s, 3H), 2.64-2.33 (m, 3H), 2.31-2.19 (m, 1H), 2.14 (d, J=0.8 Hz, 3H), 1.80-1.67 (m, 1H), 1.59-1.20 (m, 5H), 1.08-0.95 (m, 1H).

### Example 824Q methyl (1r,4R)-4-[(3-chlorophenyl)(trifluoroacetyl)amino]-6′-ethoxy-2′-{(2R)-3-[(4-methoxyphenyl)methoxy]-2-methylpropyl}-5′-methylspiro[cyclohexane-1,1′-indene]-4-carboxylate

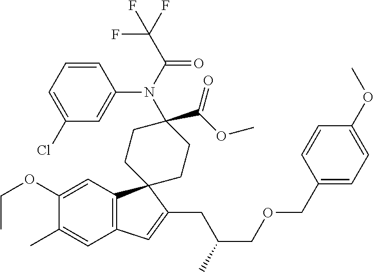 [SMILES: CCOc1cc2c(cc1C)C=C(C[C@@H](C)COCc1ccc(OC)cc1)[C@]21CC[C@@](C(=O)OC)(N(C(=O)C(F)(F)F)c2cccc(Cl)c2)CC1]

Using General procedure 27b with Example 824P as the appropriate 2-bromo-indene and Preparation 3d as the appropriate zinc reagent, Example 824Q was obtained as an off-white foam. LRMS calculated for C₃₉H₄₃ClF₃NO₆: 713; found: 714 (M+H).

### Example 824R (1r,4R)-4-(3-chloroanilino)-6′-ethoxy-2′-{(2R)-3-[(4-methoxyphenyl)methoxy]-2-methylpropyl}-5′-methylspiro[cyclohexane-1,1′-indene]-4-carboxylic acid

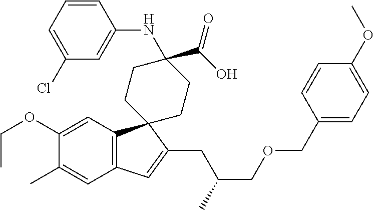 [SMILES: CCOc1cc2c(cc1C)C=C(C[C@@H](C)COCc1ccc(OC)cc1)[C@]21CC[C@](Nc2cccc(Cl)c2)(C(=O)O)CC1]

Using General procedure 33b and Example 824Q as the appropriate ester, Example 824R was obtained as a colourless gum. LRMS calculated for C₃₆H₄₂ClNO₅: 603; found: 604 (M+H).

### Example 824S methyl (1r,4R)-4-(3-chloroanilino)-6′-ethoxy-2′-{(2R)-3-[(4-methoxyphenyl)methoxy]-2-methylpropyl}-5′-methylspiro[cyclohexane-1,1′-indene]-4-carboxylate

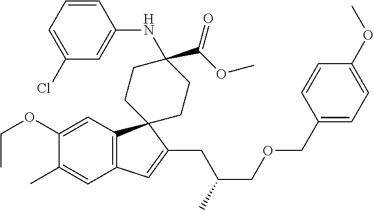 [SMILES: CCOc1cc2c(cc1C)C=C(C[C@@H](C)COCc1ccc(OC)cc1)[C@]21CC[C@](Nc2cccc(Cl)c2)(C(=O)OC)CC1]

Using General procedure 17a and Example 824R as the appropriate amino acid, Example 824S was obtained as a colourless gum. LRMS calculated for C₃₇H₄₄ClNO₅: 617; found: 618 (M+H).

### Example 824T methyl (1r,4R)-4-(3-chloroanilino)-6′-ethoxy-2′-[(2R)-3-hydroxy-2-methylpropyl]-5′-methylspiro[cyclohexane-1,1′-indene]-4-carboxylate

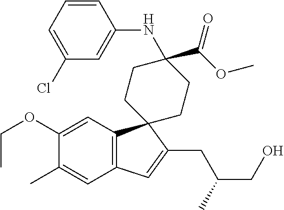 [SMILES: CCOc1cc2c(cc1C)C=C(C[C@@H](C)CO)[C@]21CC[C@](Nc2cccc(Cl)c2)(C(=O)OC)CC1]

Using General procedure 28b and Example 824S as the appropriate PMB derivative, Example 824T was obtained as a colourless gum. LRMS calculated for C₂₉H₃₆ClNO₄: 497; found: 498 (M+H).

### Example 824U methyl (1r,4R)-4-(3-chloroanilino)-6′-ethoxy-2′-[(2R)-3-hydroxy-2-methylpropyl]-5′-methyl-2′,3′-dihydrospiro[cyclohexane-1,1′-indene]-4-carboxylate

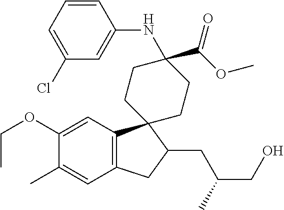

Using General procedure 19 and Example 824T as the appropriate indene, Example 824U was obtained as a mixture of diastereoisomers. LRMS calculated for C₂₉H₃₈ClNO₄: 499; found: 500 (M+H).

### Example 824V methyl (1r,4R)-4-(3-chloroanilino)-6′-ethoxy-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

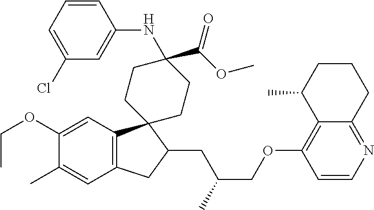

Using General procedure 30a with Example 824U as the appropriate indane and Preparation 2a1 as the appropriate alcohol, Example 824V was obtained as a mixture of diastereoisomers. LRMS calculated for C₃₉H₄₉ClN₂O₄: 644; found: 645 (M+H).

### Example 824 (1r,4R)-4-(3-chloroanilino)-6′-ethoxy-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

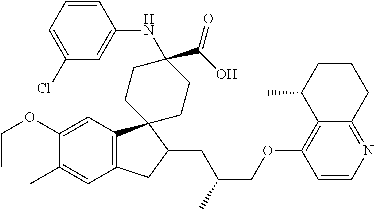

Using General procedure 33a and Example 824V as the appropriate ester, Example 824 was obtained as a mixture of diastereoisomers. ¹H NMR (500 MHz, DMSO-d₆) δ ppm: 12.76 (br s, 1H), 8.14/8.13 (d, 1H), 7.08-6.38 (m, 4H), 6.96-6.88 (s, 2H), 6.77/6.76 (d, 1H), 6.05 (br s, 1H), 4.00/3.88/3.86/3.84 (dd+dd, 2H), 3.98 (q, 2H), 3.04 (m, 1H), 2.95-1.13 (m, 20H), 2.09/2.08 (s, 3H), 1.33 (t, 3H), 1.10/1.04 (d, 3H), 1.09/1.04 (d, 3H). HRMS calculated for C₃₈H₄₇N₂O₄Cl: 630.3224; found: 631.3302 and 631.3304 (M+H).

### Example 835

### Example 835A methyl (1s,4s)-2′-bromo-4-(3-chloroanilino)-5′,6′-dimethoxyspiro[cyclohexane-1,1′-indene]-4-carboxylate

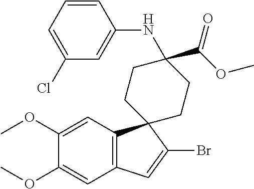

Using General procedure 17b and Example 136 as the appropriate amino acid, Example 835A was obtained. ¹H NMR (500 MHz, DMSO-d₆) δ ppm: 7.33 (s, 1H), 7.09 (t, 1H), 7.04 (s, 1H), 6.92 (s, 1H), 6.60 (dd, 1H), 6.59 (t, 1H), 6.49 (s, 1H), 6.46 (dd, 1H), 3.81 (s, 3H), 3.77 (s, 3H), 3.69 (s, 3H), 2.47/2.25 (t+d, 4H), 2.16/0.97 (t+d, 4H). HRMS calculated for C₂₄H₂₅NO₄ClBr: 505.0656; found: 506.0714 (M+H).

### Example 835B methyl (1r,4R)-4-(3-chloroanilino)-5′,6′-dimethoxy-2′-{(2R)-3-[(4-methoxyphenyl)methoxy]-2-methylpropyl}spiro[cyclohexane-1,1′-indene]-4-carboxylate

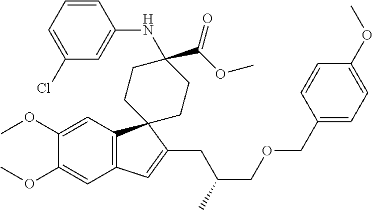 [SMILES: COc1ccc(COC[C@H](C)CC2=Cc3cc(OC)c(OC)cc3[C@]23CC[C@@](Nc2cccc(Cl)c2)(C(=O)OC)CC3)cc1]

Using General procedure 27b and Example 835A as the appropriate 2-bromo-indene derivative and Preparation 3d as the appropriate Zn reagent, Example 835B was obtained. ¹H NMR (500 MHz, DMSO-d₆) δ ppm: 7.30 (s, 1H), 7.22 (d, 2H), 7.09 (t, 1H), 6.94 (s, 1H), 6.86 (d, 2H), 6.61 (t, 1H), 6.59 (dd, 1H), 6.46 (dd, 1H), 6.41 (s, 1H), 6.33 (s, 1H), 4.39/4.36 (d+d, 2H), 3.78 (s, 3H), 3.75 (s, 3H), 3.72 (s, 3H), 3.69 (s, 3H), 3.33/3.27 (dd+dd, 2H), 2.45/2.17 (t+d, 4H), 2.31/1.94 (dd+dd, 2H), 2.13 (m, 1H), 2.06/0.85 (t+d, 4H), 0.93 (d, 3H). HRMS calculated for C₃₆H₄₂NO₆Cl: 619.2701; found: 620.2751 (M+H).

### Example 835C methyl (1r,4R)-4-(3-chloroanilino)-5′,6′-dimethoxy-2′-{(2R)-3-[(4-methoxyphenyl)methoxy]-2-methylpropyl}-2′,3′-dihydrospiro[cyclohexane-1,1′-indene]-4-carboxylate

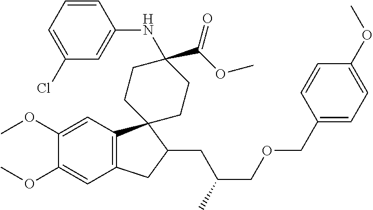

Using General procedure 19 and Example 835B as the appropriate indene, Example 835C was obtained as a mixture of diastereoisomers. ¹H NMR (500 MHz, DMSO-d₆) δ ppm: 7.21/7.20 (d/d, 2H), 7.06/7.05 (t/t, 1H), 6.95/6.94 (s/s, 1H), 6.87/6.80 (d/d, 2H), 6.82/6.81 (s/s, 1H), 6.60/6.58 (t/t, 1H), 6.56 (dd/dd, 1H), 6.46/6.44 (dd/dd, 1H), 6.32/6.30 (s/s, 1H), 4.39/4.38/4.35/4.34 (d+d/d+d, 2H), 3.73 (s, 3H), 3.72/3.69 (s/s, 3H), 3.71 (s, 3H), 3.65 (s, 3H), 3.33/3.25/3.22/3.15 (dd+dd/dd+dd, 2H), 2.86/2.85/2.44/2.41 (dd+dd/dd+dd, 2H), 2.47-1.00 (m, 8H), 2.05 (m, 1H), 1.76 (m, 1H), 1.48/1.33/1.13/1.05 (m+m/m+m, 2H), 0.94/0.88 (d/d, 3H). HRMS calculated for C₃₆H₄₄NO₆Cl: 621.2857; found: 622.2922 (M+H).

### Example 835D methyl (1r,2′S,4S)-4-(3-chloroanilino)-2′-[(2R)-3-hydroxy-2-methylpropyl]-5′,6′-dimethoxy-2′,3′-dihydrospiro[cyclohexane-1,1′-indene]-4-carboxylate

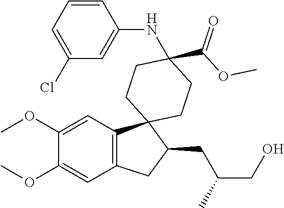 [SMILES: COc1cc2c(cc1OC)[C@]1(CC[C@@](Nc3cccc(Cl)c3)(C(=O)OC)CC1)[C@@H](C[C@@H](C)CO)C2]

Using General procedure 28b and Example 835C as the appropriate PMB derivative, a mixture of diastereoisomers was obtained. The diastereoisomers were separated via flash chromatography using hexane and EtOAc as eluents. The diastereoisomer eluting later was collected as Example 835D. ¹H NMR (500 MHz, DMSO-d₆) δ ppm: 7.06 (t, 1H), 6.94 (s, 1H), 6.83 (s, 1H), 6.57 (t, 1H), 6.56 (dm, 1H), 6.43 (dm, 1H), 6.30 (s, 1H), 4.45 (t, 1H), 3.74 (s, 3H), 3.71 (s, 3H), 3.65 (s, 3H), 3.22 (t, 2H), 2.86/2.41 (dd+dd, 2H), 2.52-1.26 (m, 8H), 2.06 (m, 1H), 1.55 (m, 1H), 1.35/1.04 (m+m, 2H), 0.85 (d, 3H). HRMS calculated for C₂₈H₃₆NO₅Cl: 501.2282; found: 502.2351 (M+H).

### Example 835 (1r,2′S,4S)-4-(3-chloroanilino)-5′,6′-dimethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

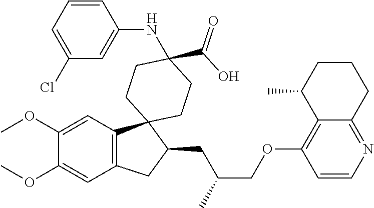 [SMILES: COc1cc2c(cc1OC)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Example 835D as the appropriate indane and Preparation 2a1 as the appropriate alcohol, Example 835 was obtained. HRMS calculated for C₃₇H₄₅N₂O₅Cl: 632.3017; found: 633.3091 (M+H).

### Example 836

### Example 836A (2E)-3-(2,3,4,5-tetrahydro-1,6-benzodioxocin-8-yl)prop-2-enoic acid

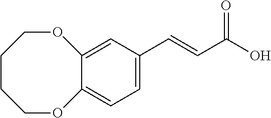 [SMILES: O=C(O)/C=C/c1ccc2c(c1)OCCCCO2]

2,3,4,5-tetrahydro-1,6-benzodioxocine-8-carbaldehyde (19.99 g, 98.8 mmol) was dissolved in pyridine (26 mL), then malonic acid (10.08 g, 96.8 mmol) and piperidine (0.52 mL, 5.24 mmol) were added. The mixture was stirred at reflux temperature for 4 h, then it was cooled. The mixture was poured onto a mixture of 150 mL ice and 50 mL cc. aq. HCl solution. The precipitation was filtered off and dried. The crude product was suspended in water (110 mL) and 5% aq. NaOH solution (300 mL), washed with DCM. The aq. phase was acidified with 5% aq. HCl solution and the white precipitation was filtered off, washed with water and dried to obtain Example 836A. ¹H NMR (500 MHz, DMSO-d₆) δ ppm: 12.25 (br s, 1H), 7.48 (d, 1H), 7.31 (br s, 1H), 7.30 (dd, 1H), 6.95 (d, 1H), 6.37 (d, 1H), 4.36 (t, 2H), 4.23 (t, 2H), 1.85 (m, 2H), 1.77 (m, 2H).

### Example 836B 3-(2,3,4,5-tetrahydro-1,6-benzodioxocin-8-yl)propanoic acid

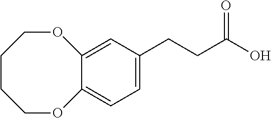 [SMILES: O=C(O)CCc1ccc2c(c1)OCCCCO2]

Example 836A (18.39 g, 78.5 mmol) was dissolved in EtOH (216 mL). 10% Pd/C (0.53 g) was added to the mixture. The autoclave was evacuated and backfilled with N₂ (×3), then evacuated and filled with H₂. The reaction mixture was stirred at 4 bar at rt overnight. Then it was filtered, washed with EtOH, and the filtrate was concentrated under reduced pressure to obtain Example 836B. ¹H NMR (500 MHz, DMSO-d₆) δ ppm: 12.09 (br s, 1H), 6.85 (d, 1H), 6.80 (br s, 1H), 6.78 (d, 1H), 4.24 (t, 2H), 4.17 (t, 2H), 2.71 (t, 2H), 2.48 (t, 1H), 1.85-1.70 (m, 4H).

### Example 836C 2,3,4,5,9,10-hexahydro-8H-indeno[5,6-b][1,4]dioxocin-8-one

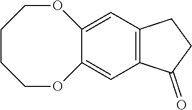 [SMILES: O=C1CCc2cc3c(cc21)OCCCCO3]

Example 836B (18.59 g, 78.7 mmol) was dissolved in dry DCM (23 mL) and DMF (0.787 mL), then oxalyl dichloride (8.1 mL, 94.44 mmol) was added dropwise over a period of 30 min. The mixture was stirred at rt overnight. TfOH (10.5 mL, 118 mmol) was added to the mixture over the period of 30 min, then it was stirred for 1 h. The mixture was poured into water (230 mL) and the organic phase was washed with water, sat. aq. NaHCO₃ solution and brine. The organic phase was dried over Na₂SO₄, filtered and the filtrate was concentrated under reduced pressure to obtain a mixture of regioisomers. The regioisomers were separated via flash chromatography using hexane and EtOAc as eluents. The regioisomer eluting earlier was collected as Example 836C. ¹H NMR (500 MHz, DMSO-d₆) δ ppm: 7.20 (s, 1H), 7.08 (s, 1H), 4.49 (t, 2H), 4.17 (m, 2H), 2.98 (m, 2H), 2.57 (m, 2H), 1.90 (m, 2H), 1.73 (m, 2H).

### Example 836D 9-bromo-2,3,4,5,9,10-hexahydro-8H-indeno[5,6-b][1,4]dioxocin-8-one

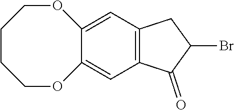 [SMILES: O=C1c2cc3c(cc2CC1Br)OCCCCO3]

Using General procedure 5 and Example 836C as the appropriate indan-1-one, Example 836D was obtained. ¹H NMR (500 MHz, DMSO-d₆) δ ppm: 7.33 (s, 1H), 7.07 (s, 1H), 4.96 (dd, 1H), 4.55 (t, 2H), 4.24-4.16 (m, 3H), 3.76 (dd, 1H), 3.20 (dd, 1H), 1.93 (m, 2H), 1.73 (m, 2H).

### Example 836E 9-bromo-2,3,4,5,9,10-hexahydro-8H-indeno[5,6-b][1,4]dioxocin-8-ol

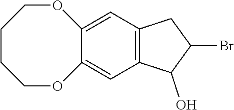 [SMILES: OC1c2cc3c(cc2CC1Br)OCCCCO3]

Using General procedure 6 and Example 836D as the appropriate bromo-indan-1-one, Example 836E was obtained as a single diastereoisomer. ¹H NMR (500 MHz, DMSO-d₆) δ ppm: 6.89 (s, 1H), 6.84 (s, 1H), 5.65 (br, 1H), 4.85-4.79 (m, 2H), 4.28-4.13 (m, 4H), 3.30 (dd, 1H), 3.09 (dd, 1H), 1.85-1.69 (m, 4H).

### Example 836F 9-bromo-2,3,4,5-tetrahydro-8H-indeno[5,6-b][1,4]dioxocine

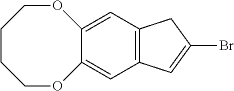 [SMILES: BrC1=Cc2cc3c(cc2C1)OCCCCO3]

Using General procedure 7 and Example 836E as the appropriate indane, Example 836F was obtained. ¹H NMR (500 MHz, DMSO-d₆) δ ppm: 7.02 (s, 1H), 6.98 (s, 1H), 6.96 (s, 1H), 4.20 (m, 4H), 3.59 (s, 2H), 1.77 (m, 4H).

### Example 836G 9″-bromo-2″,3″,4″,5″-tetrahydrodispiro[[1,3]dioxolane-2,1′-cyclohexane-4′,8″-indeno[5,6-b][1,4]dioxocine]

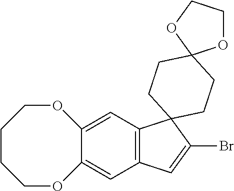 [SMILES: BrC1=Cc2cc3c(cc2C12CCC1(CC2)OCCO1)OCCCCO3]

Using General procedure 8a and Example 836F as the appropriate indene, Example 836G was obtained. ¹H NMR (500 MHz, DMSO-d₆) δ ppm: 7.21 (s, 1H), 6.95 (s, 1H), 6.91 (s, 1H), 4.24 (m, 4H), 3.95 (m, 4H), 2.08 (td, 2H), 2.02 (td, 2H), 1.84 (d, 2H), 1.79 (m, 4H), 1.16 (d, 2H).

### Example 836H 9′-bromo-2′,3′,4′,5′-tetrahydrospiro[cyclohexane-1,8′-indeno[5,6-b][1,4]dioxocin]-4-one

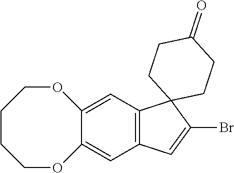 [SMILES: O=C1CCC2(CC1)C(Br)=Cc1cc3c(cc12)OCCCCO3]

Using General procedure 9 and Example 836G as the appropriate ketal, Example 836H was obtained. ¹H NMR (500 MHz, DMSO-d₆) δ ppm: 7.54 (s, 1H), 6.98 (s, 1H), 6.97 (s, 1H), 4.29-4.17 (m, 4H), 2.90/2.45 (m+m, 4H), 2.17/1.56 (m+m, 4H), 1.85-1.73 (m, 4H). HRMS calculated for C₁₈H₁₉O₃Br: 362.0518; found: 363.0592 (M+H).

### Example 836I 9″-bromo-2″,3″,4″,5″-tetrahydrodispiro[imidazolidine-4,1′-cyclohexane-4′,8″-indeno[5,6-b][1,4]dioxocine]-2,5-dione

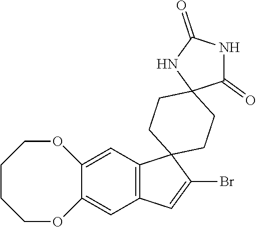 [SMILES: O=C1NC(=O)C2(CCC3(CC2)C(Br)=Cc2cc4c(cc23)OCCCCO4)N1]

Using General procedure 14 and Example 836H as the appropriate ketone, Example 8361 was obtained as a mixture of diastereoisomers. ¹H NMR (500 MHz, DMSO-d₆) δ ppm: 10.80/10.63 (br s, 1H), 8.93/8.46 (s, 1H), 7.56/7.31 (s, 1H), 6.97/6.91 (s, 1H), 6.92 (s, 1H) 4.36-4.15 (m, 4H), 2.40-1.08 (m, 8H), 1.88-1.74 (m, 4H). HRMS calculated for C₂₀H₂₁N₂O₄Br: 432.0685; found: 433.0757 and 433.0759 (M+H).

### Example 836J 4-amino-9′-bromo-2′,3′,4′,5′-tetrahydrospiro[cyclohexane-1,8′-indeno[5,6-b][1,4]dioxocine]-4-carboxylic acid

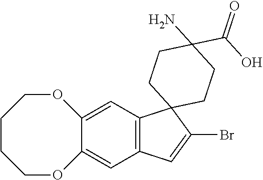 [SMILES: NC1(C(=O)O)CCC2(CC1)C(Br)=Cc1cc3c(cc12)OCCCCO3]

Using General procedure 15 and Example 8361 as the appropriate hydantoin Example 836J was obtained as a mixture of diastereoisomers. ¹H NMR (500 MHz, DMSO-d₆) δ ppm: 7.66/7.63 (s, 1H), 7.26 (br s, 3H), 6.93/6.91 (s, 1H), 6.88/6.86 (s, 1H), 4.34-4.12 (m, 4H), 2.62-0.92 (m, 8H), 1.88-1.70 (m, 4H). HRMS calculated for C₁₉H₂₂NO₄Br: 407.0732; found: 408.0806 (M+H).

### Example 836K 9′-bromo-4-(3-chloroanilino)-2′,3′,4′,5′-tetrahydrospiro[cyclohexane-1,8′-indeno[5,6-b][1,4]dioxocine]-4-carboxylic acid

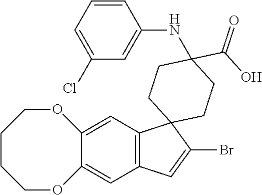 [SMILES: O=C(O)C1(Nc2cccc(Cl)c2)CCC2(CC1)C(Br)=Cc1cc3c(cc12)OCCCCO3]

Using General procedure 16 and Example 836J as the appropriate amino acid and 1-chloro-3-iodo-benzene as the appropriate iodobenzene, Example 836K was obtained as a mixture of diastereoisomers. ¹H NMR (500 MHz, DMSO-d₆) δ ppm: 12.78 (br s, 1H), 7.29/7.28 (s, 1H), 7.09 (t, 1H), 6.96/6.94 (s, 1H), 6.91/6.90 (s, 1H), 6.71/6.61 (t, 1H), 6.63/6.54 (dm, 1H), 6.59/6.57 (dm, 1H), 6.39 (br s, 1H) 4.33-4.19 (m, 4H), 2.65-0.90 (m, 8H), 1.91-1.70 (m, 4H). HRMS calculated for C₂₅H₂₅NO₄ClBr: 517.0656; found: 518.0731 and 518.0732 (M+H).

### Example 836L methyl (1s,4s)-9′-bromo-4-(3-chloroanilino)-2′,3′,4′,5′-tetrahydrospiro[cyclohexane-1,8′-indeno[5,6-b][1,4]dioxocine]-4-carboxylate

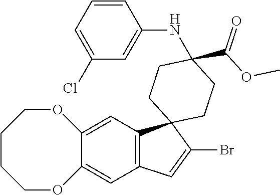 [SMILES: COC(=O)[C@]1(Nc2cccc(Cl)c2)CC[C@]2(CC1)C(Br)=Cc1cc3c(cc12)OCCCCO3]

Using General procedure 17b and Example 836K as the appropriate amino acid, a mixture of diastereoisomers was obtained. The diastereoisomers were separated by flash chromatography using heptane and EtOAC as eluents. The diastereoisomer eluting later was collected as Example 836L. ¹H NMR (500 MHz, DMSO-d₆) δ ppm: 7.27 (s, 1H), 7.09 (t, 1H), 6.96 (s, 1H), 6.90 (s, 1H), 6.60 (t, 1H), 6.59 (dm, 1H), 6.48 (s, 1H), 6.46 (dm, 1H), 4.33-4.18 (m, 4H), 3.68 (s, 3H), 2.33/2.24 (m+m, 4H), 2.16/0.98 (m+m, 4H), 1.89-1.72 (m, 4H). HRMS calculated for C₂₆H₂₇NO₄ClBr: 531.0812; found: 532.0892 (M+H).

### Example 836M methyl (1r,4R)-4-(3-chloroanilino)-9′-{(2R)-3-[(4-methoxyphenyl)methoxy]-2-methylpropyl}-2′,3′,4′,5′-tetrahydrospiro[cyclohexane-1,8′-indeno[5,6-b][1,4]dioxocine]-4-carboxylate

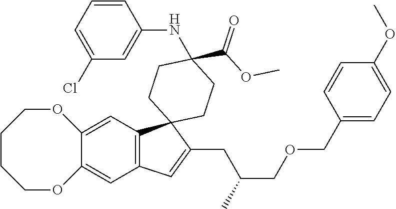 [SMILES: COc1ccc(COC[C@H](C)CC2=Cc3cc4c(cc3[C@]23CC[C@@](Nc2cccc(Cl)c2)(C(=O)OC)CC3)OCCCCO4)cc1]

Using General procedure 27b and Example 836L as the appropriate 2-bromo-indene derivative and Preparation 3d as the appropriate Zn reagent, Example 836M was obtained. ¹H NMR (500 MHz, DMSO-d₆) δ ppm: 7.21 (s, 1H), 7.20 (d, 2H), 7.08 (t, 1H), 6.85 (d, 2H), 6.84 (s, 1H), 6.62 (t, 1H), 6.59 (dd, 1H), 6.46 (dd, 1H), 6.40 (s, 1H), 6.31 (s, 1H), 4.39/4.35 (d+d, 2H), 4.23 (t, 2H), 4.21 (t, 2H), 3.72 (s, 3H), 3.68 (s, 3H), 3.32/3.26 (d+d, 2H), 2.35-0.82 (m, 8H), 2.30/1.94 (dd+dd, 2H), 2.11 (m, 1H), 1.80 (m, 4H), 0.92 (d, 3H). HRMS calculated for C₃₈H₄₄NO₆Cl: 645.2857; found: 646.2933 (M+H).

### Example 836N methyl (1r,4R)-4-(3-chloroanilino)-9′-{(2R)-3-[(4-methoxyphenyl)methoxy]-2-methylpropyl}-2′,3′,4′,5′,9′,10′-hexahydrospiro[cyclohexane-1,8′-indeno[5,6-b][1,4]dioxocine]-4-carboxylate

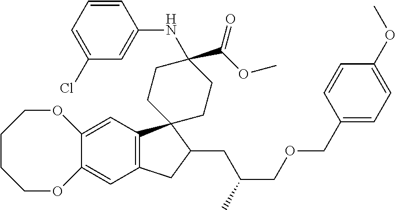

Using General procedure 19 and Example 836M as the appropriate indene (and 50° C. and 6 bar), Example 836N was obtain as a mixture of diastereoisomers. ¹H NMR (500 MHz, DMSO-d₆) δ ppm: 7.20 (m, 2H), 7.06 (t, 1H), 6.93 (s, 1H), 6.80 (m, 2H), 6.76 (s, 1H), 6.60 (t, 1H), 6.57 (dm, 1H), 6.45 (dm, 1H), 6.31 (s, 1H), 4.38/4.33 (d+d, 2H), 4.26-4.12 (m, 4H), 3.69 (s, 3H), 3.64 (s, 3H), 3.31/3.23 (dd+dd, 2H), 2.81/2.41 (dd+dd, 2H), 2.45-1.16 (m, 8H), 2.00 (m, 1H), 1.77 (m, 5H), 1.47/1.05 (m+m, 2H), 0.93 (d, 3H). HRMS calculated for C₃₈H₄₆NO₆Cl: 647.3014; found: 648.3059 (M+H).

### Example 8360 methyl (1r,4S,9′S)-4-(3-chloroanilino)-9′-[(2R)-3-hydroxy-2-methylpropyl]-2′,3′,4′,5′,9′,10′-hexahydrospiro[cyclohexane-1,8′-indeno[5,6-b][1,4]dioxocine]-4-carboxylate

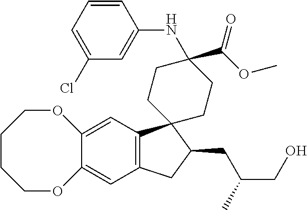 [SMILES: COC(=O)[C@]1(Nc2cccc(Cl)c2)CC[C@@]2(CC1)c1cc3c(cc1C[C@@H]2C[C@@H](C)CO)OCCCCO3]

Using General procedure 28b and Example 836N as the appropriate PMB derivative, a mixture of diastereoisomers was obtained. The diastereoisomers were separated by prep RP-HPLC using 25 mM aq. NH₄HCO₃ solution and MeCN as eluents. The diastereoisomer eluting earlier was collected as Example 8360. ¹H NMR (500 MHz, DMSO-d₆) δ ppm: 7.06 (t, 1H), 6.94 (s, 1H), 6.78 (s, 1H), 6.58 (t, 1H), 6.56 (dd, 1H), 6.43 (dd, 1H), 6.29 (s, 1H), 4.45 (t, 1H), 4.21 (m, 2H), 4.18 (m, 2H), 3.64 (s, 3H), 3.21 (t, 2H), 2.82/2.38 (dd+dd, 2H), 2.40-1.23 (m, 8H), 2.03 (m, 1H), 1.77 (m, 4H), 1.53 (m, 1H), 1.35/1.04 (t+t, 2H), 0.83 (d, 3H). HRMS calculated for C₃₀H₃₈NO₅Cl: 527.2438; found: 528.2508 (M+H).

### Example 836 (1r,4S,9′S)-4-(3-chloroanilino)-9′-[(2R)-2-methyl-3-{[(5R)-5-methyl-5,6,7,8-tetrahydroquinolin-4-yl]oxy}propyl]-2′,3′,4′,5′,9′,10′-hexahydrospiro[cyclohexane-1,8′-indeno[5,6-b][1,4]dioxocine]-4-carboxylic acid

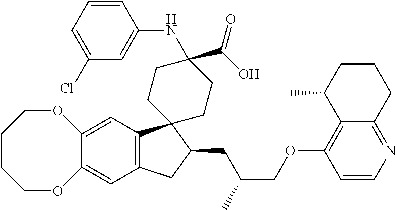 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCCCCO3]

Using General procedure 32 and Example 8360 as the appropriate indane and Preparation 2a1 as the appropriate alcohol, Example 836 was obtained. ¹H NMR (500 MHz, DMSO-d₆) δ ppm: 12.69 (br s, 1H), 8.59 (d, 1H), 7.41 (d, 1H), 7.04 (t, 1H), 6.95 (s, 1H), 6.79 (s, 1H), 6.60 (t, 1H), 6.54 (dm, 1H), 6.50 (dm, 1H), 6.23 (br s, 1H), 4.27-4.10 (m, 6H), 3.11 (m, 1H), 2.95/2.86 (m+m, 2H), 2.90/2.43 (dd+dd, 2H), 2.45-1.22 (m, 16H), 2.11 (m, 1H), 2.04 (m, 1H), 1.46/1.34 (m+m, 2H), 1.09 (d, 3H), 1.06 (d, 3H). HRMS calculated for C₃₉H₄₇N₂O₅Cl: 658.3173; found: 659.3243 (M+H).

### Example 837

### Example 837A (2E)-3-(3,4-dihydro-2H-1,5-benzodioxepin-7-yl)prop-2-enoic acid

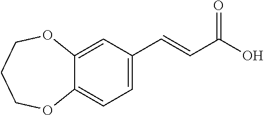 [SMILES: O=C(O)/C=C/c1ccc2c(c1)OCCCO2]

3,4-dihydro-2H-1,5-benzodioxepine-7-carbaldehyde (94.98 g, 533 mmol) was dissolved in pyridine (140 mL), then malonic acid (54.35 g, 522 mmol) and piperidine (2.8 mL, 28.2 mmol) were added. The mixture was stirred at reflux temperature for 4 h, then it was cooled. The mixture was poured onto a mixture of 1.2 L ice and 800 mL cc. aq. HCl solution. The precipitation was filtered off and dried. The crude product was suspended in water (570 mL) and 5% aq. NaOH solution (1520 mL), washed with DCM. The aq. phase was acidified with 5% aq. HCl solution and the white precipitation was filtered off, washed with water and dried to obtain Example 837A. ¹H NMR (500 MHz, DMSO-d₆) δ ppm: 12.29 (s, 1H), 7.48 (d, 1H), 7.30 (d, 1H), 7.27 (dd, 1H), 6.97 (d, 1H), 6.39 (d, 1H), 4.20-4.12 (m, 4H), 2.12 (m, 2H). HRMS calculated for C₁₂H₁₂O₄: 220.0736; found: 220.0741 (M).

### Example 837B 3-(3,4-dihydro-2H-1,5-benzodioxepin-7-yl)propanoic acid

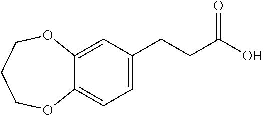 [SMILES: O=C(O)CCc1ccc2c(c1)OCCCO2]

Example 837A (85.2 g, 387 mmol) was dissolved in EtOH (1 L). 10% Pd/C (2.61 g) was added to the mixture. The flask was evacuated and backfilled with N₂ (×3), then evacuated and filled with H₂. The reaction mixture was stirred at rt overnight. Then it was filtered, washed with EtOH, and the filtrate was concentrated under reduced pressure to obtain Example 837B. ¹H NMR (500 MHz, DMSO-d₆) δ ppm: 12.10 (s, 1H), 6.85 (d, 1H), 6.82 (d, 1H), 6.77 (dd, 1H), 4.10-4.02 (m, 4H), 2.71 (t, 2H), 2.47 (t, 2H), 2.06 (m, 2H). HRMS calculated for C₁₂H₁₄O₄: 222.0892; found: 221.0822 (M−H).

### Example 837C 3,4,8,9-tetrahydro-2H,7H-indeno[5,6-b][1,4]dioxepin-7-one

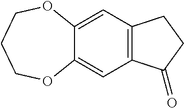 [SMILES: O=C1CCc2cc3c(cc21)OCCCO3]

Example 837B (104 g, 468 mmol) was dissolved in dry DCM (1370 mL) and DMF (4.68 mL), then oxalyl dichloride (48.2 mL, 561 mmol) was added dropwise over a period of 1 h. The mixture was stirred at rt overnight. TfOH (62.1 mL, 702 mmol) was added to the mixture over the period of 1 h, then it was stirred for 1 h. The mixture was poured into water (1370 mL) and the organic phase was washed with water, sat. aq. NaHCO₃ solution and brine. The organic phase was dried over Na₂SO₄, filtered and the filtrate was concentrated under reduced pressure to obtain Example 837C. ¹H NMR (500 MHz, DMSO-d₆) δ ppm: 7.12 (s, 1H), 7.11 (s, 1H), 4.25 (t, 2H), 4.16 (t, 2H), 2.97 (t, 2H), 2.58 (m, 2H), 2.15 (m, 2H). HRMS calculated for C₁₂H₁₂O₃: 204.0786; found: 205.0860 (M+H).

### Example 837D 8-bromo-3,4,8,9-tetrahydro-2H,7H-indeno[5,6-b][1,4]dioxepin-7-one

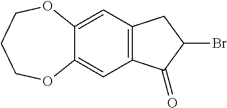 [SMILES: O=C1c2cc3c(cc2CC1Br)OCCCO3]

Using General procedure 5 and Example 837C as the appropriate indan-1-one, Example 837D was obtained. ¹H NMR (500 MHz, DMSO-d₆) δ ppm: 7.24 (s, 1H), 7.11 (s, 1H), 4.97 (dd, 1H), 4.35/4.31 (t+m, 4H), 3.75/3.19 (dd+dd, 2H), 2.19 (m, 2H). HRMS calculated for C₁₂H₁₁O₃Br: 281.9822; found: 282.9966 (M+H).

### Example 837E 8-bromo-3,4,8,9-tetrahydro-2H,7H-indeno[5,6-b][1,4]dioxepin-7-ol

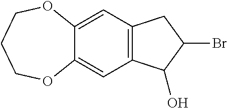 [SMILES: OC1c2cc3c(cc2CC1Br)OCCCO3]

Using General procedure 6 and Example 837D as the appropriate bromo-indan-1-one, Example 837E was obtained. ¹H NMR (500 MHz, DMSO-d₆) δ ppm: 6.88 (s, 1H), 6.85 (s, 1H), 4.82 (m, 2H), 4.12-4.05 (m, 4H), 3.29/3.08 (dd+d, 2H), 2.07 (m, 2H). HRMS calculated for C₁₂H₁₃O₃Br: 284.0048; found: 284.0025 (M).

### Example 837F 8-bromo-3,4-dihydro-2H,7H-indeno[5,6-b][1,4]dioxepine

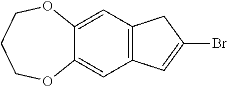 [SMILES: BrC1=Cc2cc3c(cc2C1)OCCCO3]

Using General procedure 7 and Example 837E as the appropriate indane, Example 837F was obtained. ¹H NMR (500 MHz, DMSO-d₆) δ ppm: 7.02 (s, 1H), 6.98 (br s, 1H), 6.96 (s, 1H), 4.08 (t, 4H), 3.59 (br s, 2H), 2.07 (m, 2H). HRMS calculated for C₁₂H₁₁O₂Br: 265.9942; found: 265.9925 (M).

### Example 837G 8″-bromo-3″,4″-dihydro-2″H-dispiro[[1,3]dioxolane-2,1′-cyclohexane-4′,7″-indeno[5,6-b][1,4]dioxepine]

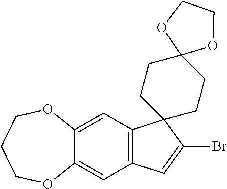 [SMILES: BrC1=Cc2cc3c(cc2C12CCC1(CC2)OCCO1)OCCCO3]

Using General procedure 8a and Example 837F as the appropriate indene, Example 837G was obtained. ¹H NMR (500 MHz, DMSO-d₆) δ ppm: 7.21 (s, 1H), 6.97 (s, 1H), 6.91 (s, 1H), 4.12/4.11 (t+t, 4H), 3.95 (m, 4H), 2.08 (m, 2H), 2.06/1.15 (m+d, 4H), 2.01/1.84 (td+d, 4H). HRMS calculated for C₁₉H₂₁O₄Br: 392.0623; found: 393.0697 (M+H).

### Example 837H 8′-bromo-3′,4′-dihydro-2′H-spiro[cyclohexane-1,7′-indeno[5,6-b][1,4]dioxepin]-4-one

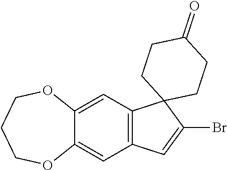 [SMILES: O=C1CCC2(CC1)C(Br)=Cc1cc3c(cc12)OCCCO3]

Using General procedure 9 and Example 837G as the appropriate ketal, Example 837H was obtained. ¹H NMR (500 MHz, DMSO-d₆) δ ppm: 7.53 (s, 1H), 6.99 (s, 1H), 6.97 (s, 1H), 4.13 (m, 4H), 2.88/2.45 (m+dm, 4H), 2.16/1.57 (td+dm, 4H), 2.10 (m, 2H). HRMS calculated for C₁₇H₁₇O₃Br: 348.0361; found: 349.0431 (M+H).

### Example 837I 8″-bromo-3″,4″-dihydro-2″H-dispiro[imidazolidine-4,1′-cyclohexane-4′,7″-indeno[5,6-b][1,4]dioxepine]-2,5-dione

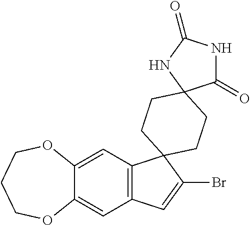 [SMILES: O=C1NC(=O)C2(CCC3(CC2)C(Br)=Cc2cc4c(cc23)OCCCO4)N1]

Using General procedure 14 and Example 837H as the appropriate ketone, Example 8371 was obtained as a mixture of diastereoisomers. ¹H NMR (500 MHz, DMSO-d₆) δ ppm: 10.81/10.60 (s, 1H), 8.95/8.46 (s, 1H), 7.59/7.33 (s, 1H), 6.97/6.92 (s, 1H), 6.92/6.91 (s, 1H), 4.19-4.07 (m, 4H), 2.42-1.06 (m, 8H), 2.09 (m, 2H). HRMS calculated for C₁₉H₁₉N₂O₄Br: 418.0528; found: 419.0600 and 419.0603 (M+H).

### Example 837J 4-amino-8′-bromo-3′,4′-dihydro-2′H-spiro[cyclohexane-1,7′-indeno[5,6-b][1,4]dioxepine]-4-carboxylic acid

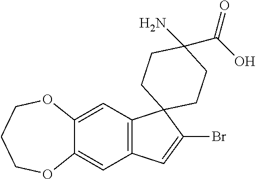 [SMILES: NC1(C(=O)O)CCC2(CC1)C(Br)=Cc1cc3c(cc12)OCCCO3]

Using General procedure 15 and Example 8371 as the appropriate hydantoin, Example 837J was obtained as a mixture of diastereoisomers. ¹H NMR (500 MHz, DMSO-d₆) δ ppm: 7.68/7.64 (s, 1H), 7.31 (br s, 3H), 6.94 (s, 1H), 6.88 (s, 1H), 4.17-4.04 (m, 4H), 2.65-0.85 (m, 8H), 2.08 (m, 2H). HRMS calculated for C₁₈H₂₀NO₄Br: 393.0576; found: 394.0649 (M+H).

### Example 837K 8′-bromo-4-(3-chloroanilino)-3′,4′-dihydro-2′H-spiro[cyclohexane-1,7′-indeno[5,6-b][1,4]dioxepine]-4-carboxylic acid

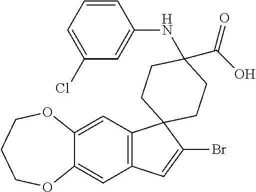 [SMILES: O=C(O)C1(Nc2cccc(Cl)c2)CCC2(CC1)C(Br)=Cc1cc3c(cc12)OCCCO3]

Using General procedure 16 and Example 837J as the appropriate amino acid and 1-chloro-3-iodo-benzene as the appropriate iodobenzene, Example 837K was obtained as a mixture of diastereoisomers. ¹H NMR (500 MHz, DMSO-d₆) δ ppm: 12.88 (br s, 1H), 7.32/7.30 (s, 1H), 7.19/7.08 (t, 1H), 6.99/6.97 (s, 1H), 6.91/6.90 (s, 1H), 6.71/6.60 (t, 1H), 6.63/6.54 (dm, 1H), 6.59/6.57 (dm, 1H), 6.39 (br s, 1H), 4.20-4.05 (m, 4H), 2.65-0.88 (m, 8H), 2.09 (m, 2H). HRMS calculated for C₂₄H₂₃NO₄ClBr: 503.0499; found: 504.0575 and 504.0576 (M+H).

### Example 837L methyl (1s,4s)-8′-bromo-4-(3-chloroanilino)-3′,4′-dihydro-2′H-spiro[cyclohexane-1,7′-indeno[5,6-b][1,4]dioxepine]-4-carboxylate

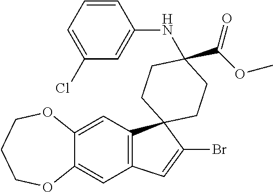 [SMILES: COC(=O)[C@]1(Nc2cccc(Cl)c2)CC[C@]2(CC1)C(Br)=Cc1cc3c(cc12)OCCCO3]

Using General procedure 17b and Example 837K as the appropriate amino acid, a mixture of diastereoisomers was obtained. The diastereoisomers were separated by flash chromatography using heptane and EtOAC as eluents. The diastereoisomer eluting later was collected as Example 837L. ¹H NMR (500 MHz, DMSO-d₆) δ ppm: 7.29 (s, 1H), 7.09 (t, 1H), 6.97 (s, 1H), 6.90 (s, 1H), 6.60 (m, 1H), 6.59 (dm, 1H), 6.48 (br s, 1H), 6.46 (dm, 1H), 4.15 (t, 2H), 4.12 (t, 2H), 3.68 (s, 3H), 2.32/2.24 (td+d, 4H), 2.16/0.97 (td+d, 4H), 2.09 (quint., 2H). HRMS calculated for C₂₅H₂₅NO₄ClBr: 517.0656; found: 518.0719 (M+H).

### Example 837M methyl (1r,4R)-4-(3-chloroanilino)-8′-{(2R)-3-[(4-methoxyphenyl)methoxy]-2-methylpropyl}-3′,4′-dihydro-2′H-spiro[cyclohexane-1,7′-indeno[5,6-b][1,4]dioxepine]-4-carboxylate

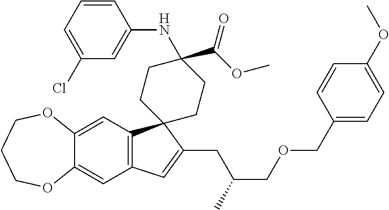 [SMILES: COc1ccc(COC[C@H](C)CC2=Cc3cc4c(cc3[C@]23CC[C@@](Nc2cccc(Cl)c2)(C(=O)OC)CC3)OCCCO4)cc1]

Using General procedure 27b and Example 837L as the appropriate 2-bromo-indene derivative and Preparation 3d as the appropriate Zn reagent, Example 837M was obtained. ¹H NMR (500 MHz, DMSO-d₆) δ ppm: 7.23 (s, 1H), 7.21 (dm, 2H), 7.09 (t, 1H), 6.86 (s, 1H), 6.85 (dm, 2H), 6.62 (t, 1H), 6.59 (dm, 1H), 6.46 (dm, 1H), 6.41 (s, 1H), 6.31 (s, 1H), 4.39/4.35 (d+d, 2H), 4.09 (m, 4H), 3.72 (s, 3H), 3.68 (s, 3H), 3.32/3.26 (dd+dd, 2H), 2.35-0.80 (m, 10H), 2.08 (m, 1H), 2.08 (m, 2H), 0.92 (d, 3H). HRMS calculated for C₃₇H₄₂NO₆Cl: 631.2701; found: 632.2775 (M+H).

### Example 837N methyl (1r,4R)-4-(3-chloroanilino)-8′-{(2R)-3-[(4-methoxyphenyl)methoxy]-2-methylpropyl}-3′,4′,8′,9′-tetrahydro-2′H-spiro[cyclohexane-1,7′-indeno[5,6-b][1,4]dioxepine]-4-carboxylate

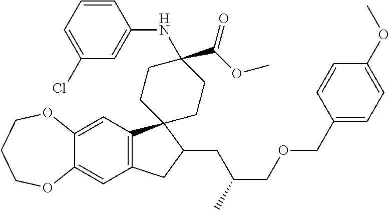

Using General procedure 19 and Example 837M as the appropriate indene, Example 837N was obtain as a mixture of diastereoisomers. LRMS calculated for C₃₇H₄₄NO₆Cl: 633.3; found: 634.3 (M+H).

### Example 8370 methyl (1r,4S,8′S)-4-(3-chloroanilino)-8′-[(2R)-3-hydroxy-2-methylpropyl]-3′,4′,8′,9′-tetrahydro-2′H-spiro[cyclohexane-1,7′-indeno[5,6-b][1,4]dioxepine]-4-carboxylate

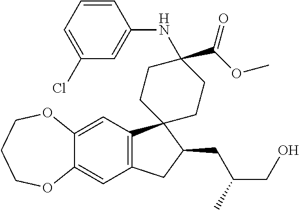 [SMILES: COC(=O)[C@]1(Nc2cccc(Cl)c2)CC[C@@]2(CC1)c1cc3c(cc1C[C@@H]2C[C@@H](C)CO)OCCCO3]

Using General procedure 28b and Example 837N as the appropriate PMB derivative, a mixture of diastereoisomers was obtained. The diastereoisomers were separated via chiral chromatography. Column: IC, 50×500 mm, 20 μm, Eluents: EtOH/heptane. The diastereoisomer eluting later was collected as Example 8370. ¹H NMR (500 MHz, DMSO-d₆) δ ppm: 7.06 (t, 1H), 6.94 (s, 1H), 6.79 (s, 1H), 6.58 (t, 1H), 6.56 (dm, 1H), 6.43 (dm, 1H), 6.29 (s, 1H), 4.45 (t, 1H), 4.14-3.98 (m, 4H), 3.64 (s, 3H), 3.26-3.17 (m, 2H), 2.82/2.38 (dd+dd, 2H), 2.41-0.98 (m, 10H), 2.06 (m, 2H), 2.03 (m, 1H), 1.53 (m, 1H), 0.83 (d, 3H). HRMS calculated for C₂₉H₃₆NO₅Cl: 513.2282; found: 514.2350 (M+H).

### Example 837 (1r,4S,8′S)-4-(3-chloroanilino)-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

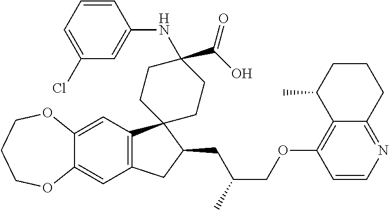 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCCCO3]

Using General procedure 32 and Example 8370 as the appropriate indane and Preparation 2a1 as the appropriate alcohol, Example 837 was obtained. ¹H NMR (500 MHz, DMSO-d₆) δ ppm: 12.68 (br s, 1H), 8.14 (d, 1H), 7.04 (t, 1H), 6.96 (s, 1H), 6.80 (s, 1H), 6.77 (d, 1H), 6.59 (t, 1H), 6.53 (dm, 1H), 6.51 (dm, 1H), 6.23 (br s, 1H), 4.14-3.98 (m, 4H), 3.90/3.84 (dd+dd, 2H), 3.06 (m, 1H), 2.88/2.42 (dd+dd, 2H), 2.76/2.65 (m+m, 2H), 2.44-1.19 (m, 12H), 2.08 (m, 1H), 2.06 (m, 2H), 1.96 (m, 1H), 1.49/1.32 (m+m, 2H), 1.06 (d, 3H), 1.02 (d, 3H). HRMS calculated for C₃₈H₄₅N₂O₅Cl: 644.3017; found: 645.3069 (M+H).

### Example 838

### Example 838A (2E)-3-(2,3-dihydro-1,4-benzodioxin-6-yl)prop-2-enoic acid

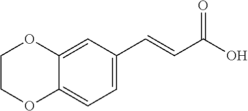 [SMILES: O=C(O)/C=C/c1ccc2c(c1)OCCO2]

2,3-dihydro-1,4-benzodioxine-6-carbaldehyde (22.65 g, 138 mmol) was dissolved in pyridine (36 mL), then malonic acid (14.07 g, 135 mmol) and piperidine (0.72 mL, 7.3 mmol) were added. The mixture was stirred at reflux temperature for 4 h, then it was cooled. The mixture was poured onto a mixture of 200 mL ice and 60 mL cc. aq. HCl solution. The precipitation was filtered off and dried. The crude product was suspended in water (150 mL) and 5% aq. NaOH solution (400 mL), washed with DCM. The aq. phase was acidified with 5% aq. HCl solution and the white precipitation was filtered off, washed with water and dried to obtain Example 838A. ¹H NMR (500 MHz, DMSO-d₆) δ ppm: 12.23 (s, 1H), 7.47 (d, 1H), 7.23 (d, 1H), 7.17 (dd, 1H), 6.87 (d, 1H), 6.35 (d, 1H), 4.30-4.22 (m, 4H). HRMS calculated for C₁₁H₁₀O₄: 206.0579; found: 205.0506 (M−H).

### Example 838B 3-(2,3-dihydro-1,4-benzodioxin-6-yl)propanoic acid

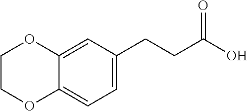 [SMILES: O=C(O)CCc1ccc2c(c1)OCCO2]

Example 838A (15.9 g, 77.1 mmol) was dissolved in EtOH (210 mL). 10% Pd/C (0.53 g) was added to the mixture. The autoclave was evacuated and backfilled with N₂ (×3), then evacuated and filled with H₂. The reaction mixture was stirred at 4 bar and at rt overnight. The reaction mixture was filtered, washed with EtOH, and the filtrate was concentrated under reduced pressure to obtain Example 838B. ¹H NMR (500 MHz, DMSO-d₆) δ ppm: 12.08 (s, 1H), 6.73 (d, 1H), 6.70 (d, 1H), 6.65 (dd, 1H), 4.22-4.15 (m, 4H), 2.68 (t, 2H), 2.46 (t, 2H). HRMS calculated for C₁₁H₁₂O₄: 208.0736; found: 207.0663 (M−H).

### Example 838C 2,3,7,8-tetrahydro-6H-indeno[5,6-b][1,4]dioxin-6-one

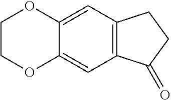 [SMILES: O=C1CCc2cc3c(cc21)OCCO3]

Example 838B (93.7 g, 450 mmol) was dissolved in dry DCM (1310 mL) and DMF (4.5 mL), then oxalyl dichloride (46.3 mL, 540 mmol) was added dropwise over a period of 2 h. The mixture was stirred at rt overnight. TfOH (59.7 mL, 675 mmol) was added to the mixture over the period of 2 h, then it was stirred for 1 h. The mixture was poured into water (1300 mL) and the organic phase was washed with water, sat. aq. NaHCO₃ solution and brine. The organic phase was dried over Na₂SO₄, filtered and the filtrate was concentrated under reduced pressure to obtain Example 838C. ¹H NMR (500 MHz, DMSO-d₆) δ ppm: 7.03 (s, 1H), 7.02 (t, 1H), 4.33/4.26 (m+m, 4H), 2.95 (m, 2H), 2.55 (m, 2H). HRMS calculated for C₁₁H₁₀O₃: 190.0630; found: 191.0703 (M+H).

### Example 837D 7-bromo-2,3,7,8-tetrahydro-6H-indeno[5,6-b][1,4]dioxin-6-one

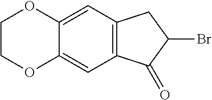 [SMILES: O=C1c2cc3c(cc2CC1Br)OCCO3]

Using General procedure 5 and Example 838C as the appropriate indan-1-one, Example 838D was obtained. ¹H NMR (500 MHz, DMSO-d₆) δ ppm: 7.16 (s, 1H), 7.03 (s, 1H), 4.95 (dd, 1H), 4.39-4.26 (m, 4H), 3.75/3.18 (dd+dd, 2H). HRMS calculated for C₁₁H₉O₃Br: 267.9735; found: 268.9807 (M+H).

### Example 838E 7-bromo-2,3,7,8-tetrahydro-6H-indeno[5,6-b][1,4]dioxin-6-ol

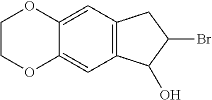 [SMILES: OC1c2cc3c(cc2CC1Br)OCCO3]

Using General procedure 6 and Example 838D as the appropriate bromo-indan-1-one, Example 838E was obtained as a single diastereoisomer. ¹H NMR (500 MHz, DMSO-d₆) δ ppm: 6.76 (s, 1H), 6.73 (s, 1H), 5.61 (d, 1H), 4.83-4.77 (m, 2H), 4.24-4.15 (m, 4H), 3.28/3.07 (dd+dd, 2H).

### Example 838F 7-bromo-2,3-dihydro-6H-indeno[5,6-b][1,4]dioxine

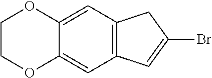 [SMILES: BrC1=Cc2cc3c(cc2C1)OCCO3]

Using General procedure 7 and Example 838E as the appropriate indane, Example 838F was obtained. ¹H NMR (500 MHz, DMSO-d₆) δ ppm: 6.95 (m, 1H), 6.92 (br s, 1H), 6.85 (s, 1H), 4.22-4.19 (m, 4H), 3.55 (m, 2H). HRMS calculated for C₁₁H₉O2Br: 251.9786; found: 251.9762 (M).

### Example 838G 7″-bromo-2″,3″-dihydrodispiro[[1,3]dioxolane-2,1′-cyclohexane-4′,6″-indeno[5,6-b][1,4]dioxine]

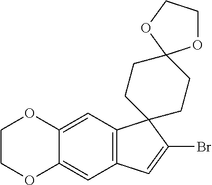 [SMILES: BrC1=Cc2cc3c(cc2C12CCC1(CC2)OCCO1)OCCO3]

Using General procedure 8a and Example 838F as the appropriate indene, Example 838G was obtained. ¹H NMR (500 MHz, DMSO-d₆) δ ppm: 7.09 (s, 1H), 6.87 (s, 1H), 6.86 (s, 1H), 4.25-4.20 (m, 4H), 3.96-3.91 (m, 4H), 2.12-1.96 (m, 4H), 1.87-1.81 (m, 2H), 1.19-1.13 (m, 2H).

### Example 838H 7′-bromo-2′,3′-dihydrospiro[cyclohexane-1,6′-indeno[5,6-b][1,4]dioxin]-4-one

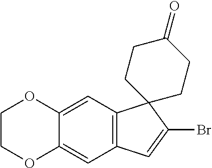 [SMILES: O=C1CCC2(CC1)C(Br)=Cc1cc3c(cc12)OCCO3]

Using General procedure 9 and Example 838G as the appropriate ketal, Example 838H was obtained. ¹H NMR (500 MHz, DMSO-d₆) δ ppm: 7.43 (s, 1H), 6.94 (s, 1H), 6.89 (s, 1H), 4.24 (m, 4H), 2.88 (ddd, 2H), 2.45 (dm, 2H), 2.16 (td, 2H), 1.57 (dm, 2H).

### Example 838I 7″-bromo-2″,3″-dihydrodispiro[imidazolidine-4,1′-cyclohexane-4′,6″-indeno[5,6-b][1,4]dioxine]-2,5-dione

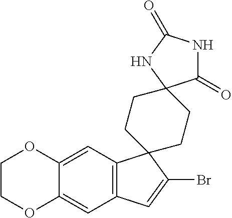 [SMILES: O=C1NC(=O)C2(CCC3(CC2)C(Br)=Cc2cc4c(cc23)OCCO4)N1]

Using General procedure 14 and Example 838H as the appropriate ketone, Example 8381 was obtained as a mixture of diastereoisomers. ¹H NMR (500 MHz, DMSO-d₆) δ ppm: 10.73 (br s, 1H), 8.87/8.45 (s/s, 1H), 7.47/7.22 (s/s, 1H), 6.89/6.88 (s/s, 1H), 6.87/6.81 (s/s, 1H), 4.24 (m, 4H), 2.30/1.90/1.70 (td+d/br, 4H), 2.07/1.53/1.12 (td+d/m, 4H). HRMS calculated for C₁₈H₁₇N₂O₄Br: 404.0372; found: 405.0446 and 405.0442 (M+H).

### Example 838J 4-amino-7′-bromo-2′,3′-dihydrospiro[cyclohexane-1,6′-indeno[5,6-b][1,4]dioxine]-4-carboxylic acid

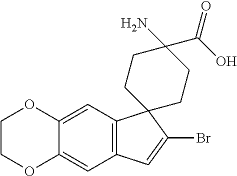 [SMILES: NC1(C(=O)O)CCC2(CC1)C(Br)=Cc1cc3c(cc12)OCCO3]

Using General procedure 15 and Example 8381 as the appropriate hydantoin, Example 838J was obtained as a mixture of diastereoisomers. ¹H NMR (500 MHz, DMSO-d₆) δ ppm: 7.82 (br s, 3H), 7.59 (s, 1H), 6.87/6.83 (4, 1H), 6.84/6.81 (s, 1H), 4.29-4.15 (m, 4H), 2.66-0.94 (m, 8H). HRMS calculated for C₁₇H₁₈NO₄Br: 379.0419; found: 405.0446 and 380.0491 (M+H).

### Example 838K 7′-bromo-4-(3-chloroanilino)-2′,3′-dihydrospiro[cyclohexane-1,6′-indeno[5,6-b][1,4]dioxine]-4-carboxylic acid

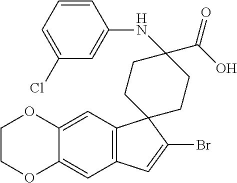 [SMILES: O=C(O)C1(Nc2cccc(Cl)c2)CCC2(CC1)C(Br)=Cc1cc3c(cc12)OCCO3]

Using General procedure 16 and Example 838J as the appropriate amino acid and 1-chloro-3-iodo-benzene as the appropriate iodobenzene, Example 838K was obtained as a mixture of diastereoisomers. ¹H NMR (500 MHz, DMSO-d₆) δ ppm: 12.65 (br s, 1H), 7.21/7.19 (s, 1H), 7.08 (t, 1H), 6.87 (s, 1H), 6.87 (s, 1H), 6.59 (t, 1H), 6.53 (dm, 2H), 6.36 (br s, 1H), 4.33-4.11 (m, 4H), 2.60-0.88 (m, 8H). HRMS calculated for C₂₃H₂₁NO₄ClBr: 489.0342; found: 490.0415 and 490.0412 (M+H).

### Example 838L methyl (1s,4s)-7′-bromo-4-(3-chloroanilino)-2′,3′-dihydrospiro[cyclohexane-1,6′-indeno[5,6-b][1,4]dioxine]-4-carboxylate

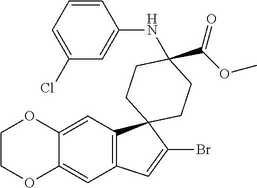 [SMILES: COC(=O)[C@]1(Nc2cccc(Cl)c2)CC[C@]2(CC1)C(Br)=Cc1cc3c(cc12)OCCO3]

Using General procedure 17b and Example 838K as the appropriate amino acid, a mixture of diastereoisomers was obtained. The diastereoisomers were separated by flash chromatography using heptane and EtOAC as eluents. The diastereoisomer eluting later was collected as Example 838L. ¹H NMR (500 MHz, DMSO-d₆) δ ppm: 7.18 (s, 1H), 7.09 (t, 1H), 6.88 (s, 1H), 6.87 (s, 1H), 6.59 (m, 2H), 6.47 (s, 1H), 6.45 (dm, 1H), 4.24 (m, 4H), 3.68 (s, 3H), 2.32/2.23 (td+d, 4H), 2.15/0.96 (td+d, 4H). HRMS calculated for C₂₄H₂₃NO₄ClBr: 503.0499; found: 504.0573 (M+H).

### Example 838M methyl (1r,4R)-4-(3-chloroanilino)-7′-{(2R)-3-[(4-methoxyphenyl)methoxy]-2-methylpropyl}-2′,3′-dihydrospiro[cyclohexane-1,6′-indeno[5,6-b][1,4]dioxine]-4-carboxylate

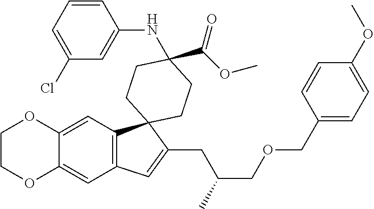 [SMILES: COc1ccc(COC[C@H](C)CC2=Cc3cc4c(cc3[C@]23CC[C@@](Nc2cccc(Cl)c2)(C(=O)OC)CC3)OCCO4)cc1]

Using General procedure 27b and Example 838L as the appropriate 2-bromo-indene derivative and Preparation 3d as the appropriate Zn reagent, Example 838M was obtained. ¹H NMR (500 MHz, DMSO-d₆) δ ppm: 7.21 (m, 2H), 7.11 (s, 1H), 7.08 (t, 1H), 6.85 (m, 2H), 6.74 (s, 1H), 6.62 (t, 1H), 6.59 (dm, 1H), 6.45 (dm, 1H), 6.40 (s, 1H), 6.28 (m, 1H), 4.39/4.35 (d+d, 2H), 4.26-4.17 (m, 4H), 3.72 (s, 3H), 3.68 (s, 3H), 3.32/3.26 (dd+dd, 2H), 2.31/2.15 (m+m, 4H), 2.29/1.92 (dd+dd, 2H), 2.10 (m, 1H), 2.04/0.82 (m+m, 4H), 0.92 (d, 3H).

### Example 838N methyl (1r,4R)-4-(3-chloroanilino)-7′-{(2R)-3-[(4-methoxyphenyl)methoxy]-2-methylpropyl}-2′,3′,7′,8′-tetrahydrospiro[cyclohexane-1,6′-indeno[5,6-b][1,4]dioxine]-4-carboxylate

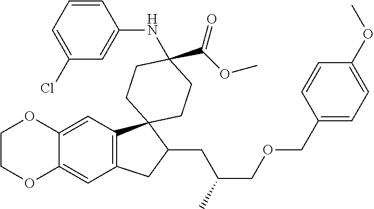

Using General procedure 19 and Example 838M as the appropriate indene, Example 838N was obtain as a mixture of diastereoisomers. LRMS calculated for C₃₆H₄₂NO₆Cl: 619.3; found: 620.3 (M+H).

### Example 8380 methyl (1r,4S,7′S)-4-(3-chloroanilino)-7′-[(2R)-3-hydroxy-2-methylpropyl]-2′,3′,7′,8′-tetrahydrospiro[cyclohexane-1,6′-indeno[5,6-b][1,4]dioxine]-4-carboxylate

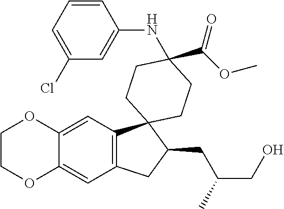 [SMILES: COC(=O)[C@]1(Nc2cccc(Cl)c2)CC[C@@]2(CC1)c1cc3c(cc1C[C@@H]2C[C@@H](C)CO)OCCO3]

Using General procedure 28b and Example 838N as the appropriate PMB derivative, a mixture of diastereoisomers was obtained. The diastereoisomers were separated via chiral chromatography. Column: AD, 100×500 mm, 20 μm, Eluents: iPrOH/heptane. The diastereoisomer eluting later was collected as Example 8380. ¹H NMR (500 MHz, DMSO-d₆) δ ppm: 7.06 (t, 1H), 6.82 (s, 1H), 6.67 (s, 1H), 6.57 (t, 1H), 6.56 (dm, 1H), 6.43 (dm, 1H), 6.29 (s, 1H), 4.44 (t, 1H), 4.24-4.11 (m, 4H), 3.64 (s, 3H), 3.21 (m, 2H), 2.80/2.36 (dd+dd, 2H), 2.43-1.19 (m, 8H), 2.00 (m, 1H), 1.53 (m, 1H), 1.33/1.03 (m+m, 2H), 0.83 (d, 3H).

### Example 838 (1r,4S,7′S)-4-(3-chloroanilino)-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

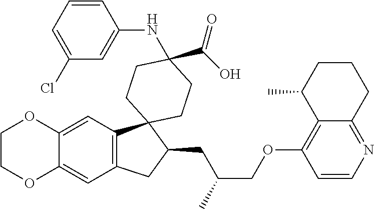 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCCO3]

Using General procedure 32 and Example 8380 as the appropriate indane and Preparation 2a1 as the appropriate alcohol, Example 838 was obtained. ¹H NMR (500 MHz, DMSO-d₆) δ ppm: 12.67 (br s, 1H), 8.14 (d, 1H), 7.03 (t, 1H), 6.83 (s, 1H), 6.77 (d, 1H), 6.68 (s, 1H), 6.58 (t, 1H), 6.53 (dd, 1H), 6.51 (dd, 1H), 6.22 (br s, 1H), 4.18 (s, 4H), 3.90/3.84 (dd+dd, 2H), 3.06 (m, 1H), 2.87/2.40 (dd+dd, 2H), 2.76/2.65 (m+m, 2H), 2.39-1.22 (m, 8H), 2.06 (m, 1H), 1.96 (m, 1H), 1.81/1.74 (m+m, 2H), 1.68/1.62 (m+m, 2H), 1.48/1.31 (t+t, 2H), 1.06 (d, 3H), 1.02 (d, 3H). HRMS calculated for C₃₇H₄₃N₂O₅Cl: 630.2861; found: 631.2917 (M+H).

### Example 839, Example 840, Example 841, Example 842, Example 843 and Example 844

### Example 839A 6′-bromo-4-(3-chloroanilino)-2′H-spiro[cyclohexane-1,5′-indeno[5,6-d][1,3]dioxole]-4-carboxylic acid

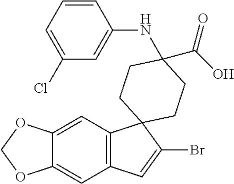 [SMILES: O=C(O)C1(Nc2cccc(Cl)c2)CCC2(CC1)C(Br)=Cc1cc3c(cc12)OCO3]

Using General procedure 16 and Preparation 5b as the appropriate amino acid and 1-chloro-3-iodo-benzene as the appropriate iodobenzene, Example 839A was obtained as a mixture of diastereoisomers. ¹H NMR (500 MHz, DMSO-d₆) δ ppm: 12.84 (br s, 1H), 7.25 (s, 1H), 7.09 (t, 1H), 6.98 (s, 1H), 6.89 (s, 1H), 6.61 (t, 1H), 6.57 (dm, 1H), 6.54 (dm, 1H), 6.39 (br s, 1H), 6.02 (s, 2H), 2.33/2.23 (m+m, 4H), 2.15/0.97 (m+m, 4H). HRMS calculated for C₂₂H₁₉NO₄ClBr: 475.0186; found: 476.0240 and 476.0248 (M+H).

### Example 839B methyl (1s,4s)-6′-bromo-4-(3-chloroanilino)-2′H-spiro[cyclohexane-1,5′-indeno[5,6-d][1,3]dioxole]-4-carboxylate

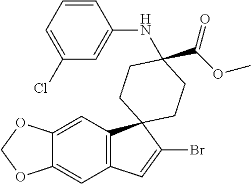 [SMILES: COC(=O)[C@]1(Nc2cccc(Cl)c2)CC[C@]2(CC1)C(Br)=Cc1cc3c(cc12)OCO3]

Using General procedure 17b and Example 839A as the appropriate amino acid, a mixture of diastereoisomers was obtained. The diastereoisomers were separated by flash chromatography using heptane and EtOAC as eluents. The diastereoisomer eluting later was collected as Example 839B. ¹H NMR (500 MHz, DMSO-d₆) δ ppm: 7.27 (s, 1H), 7.09 (t, 1H), 6.97 (s, 1H), 6.89 (s, 1H), 6.61 (t, 1H), 6.59 (dm, 1H), 6.47 (dm, 2H), 6.02 (s, 2H), 3.68 (s, 3H), 2.34 (td, 2H), 2.25 (d, 2H), 2.16 (td, 2H), 0.97 (d, 2H). HRMS calculated for C₂₃H₂₁NO₄ClBr: 489.0342; found: 490.0400 (M+H).

### Example 839C methyl (1r,4R)-4-(3-chloroanilino)-6′-{(2R)-3-[(4-methoxyphenyl)methoxy]-2-methylpropyl}-2′H-spiro[cyclohexane-1,5′-indeno[5,6-d][1,3]dioxole]-4-carboxylate

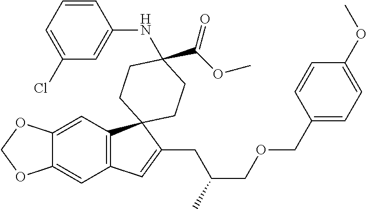 [SMILES: COc1ccc(COC[C@H](C)CC2=Cc3cc4c(cc3[C@]23CC[C@@](Nc2cccc(Cl)c2)(C(=O)OC)CC3)OCO4)cc1]

Using General procedure 27b and Example 839B as the appropriate 2-bromo-indene derivative and Preparation 3d as the appropriate Zn reagent, Example 839C was obtained. ¹H NMR (500 MHz, DMSO-d₆) δ ppm: 7.21 (m, 2H), 7.19 (s, 1H), 7.08 (t, 1H), 6.87 (s, 1H), 6.85 (m, 2H), 6.63 (t, 1H), 6.59 (dm, 1H), 6.47 (dm, 1H), 6.40 (s, 1H), 6.31 (t, 1H), 5.97 (s, 2H), 4.39/3.35 (d+d, 2H), 3.72 (s, 3H), 3.68 (s, 3H), 3.32/3.26 (dd+dd, 2H), 2.41-078 (m, 8H), 2.29/1.93 (m+m, 2H), 2.11 (m, 1H), 0.92 (d, 3H). HRMS calculated for C₃₅H₃₈NO₆Cl: 603.2388; found: 604.2448 (M+H).

### Example 839D methyl (1r,4R)-4-(3-chloroanilino)-6′-{(2R)-3-[(4-methoxyphenyl)methoxy]-2-methylpropyl}-6′,7′-dihydro-2′H-spiro[cyclohexane-1,5′-indeno[5,6-d][1,3]dioxole]-4-carboxylate

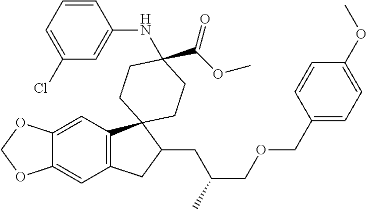

Using General procedure 19 and Example 839C as the appropriate indene, Example 839D was obtain as a mixture of diastereoisomers. ¹H NMR (500 MHz, DMSO-d₆) δ ppm: 7.20 (m, 2H), 7.10-6.42 (m, 4H), 6.87 (s, 1H), 6.87/6.80 (m, 2H), 6.75 (s, 1H), 6.30/6.29 (s, 1H), 5.99-5.90 (s, 2H), 4.42-4.30 (d+d, 2H), 3.72/3.68 (s, 3H), 3.64 (s, 3H), 3.36-3.16 (m, 2H), 2.83/2.40 (dd+dd, 2H), 2.47-0.96 (m, 11H), 2.06 (m, 1H), 0.93/0.88 (d, 3H). HRMS calculated for C₃₅H₄₀NO₆Cl: 605.2544; found: 606.2603 (M+H).

### Example 839E methyl (1r,4S,6′S)-4-(3-chloroanilino)-6′-[(2R)-3-hydroxy-2-methylpropyl]-6′,7′-dihydro-2′H-spiro[cyclohexane-1,5′-indeno[5,6-d][1,3]dioxole]-4-carboxylate

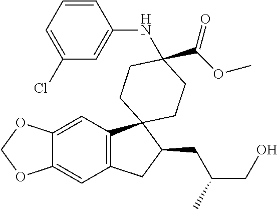 [SMILES: COC(=O)[C@]1(Nc2cccc(Cl)c2)CC[C@@]2(CC1)c1cc3c(cc1C[C@@H]2C[C@@H](C)CO)OCO3]

and

### Example 839F methyl (1r,4R,6′R)-4-(3-chloroanilino)-6′-[(2R)-3-hydroxy-2-methylpropyl]-6′,7′-dihydro-2′H-spiro[cyclohexane-1,5′-indeno[5,6-d][1,3]dioxole]-4-carboxylate

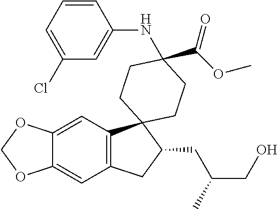 [SMILES: COC(=O)[C@]1(Nc2cccc(Cl)c2)CC[C@@]2(CC1)c1cc3c(cc1C[C@H]2C[C@@H](C)CO)OCO3]

Using General procedure 28b and Example 839D as the appropriate PMB derivative a mixture of diastereoisomers was obtained. The diastereoisomers were separated via prep RP-HPLC using 25 mM aq. NH₄HCO₃ solution and MeCN as eluents. The diastereoisomer eluting earlier was collected as Example 839E. ¹H NMR (500 MHz, DMSO-d₆) δ ppm: 7.06 (t, 1H), 6.87 (s, 1H), 6.76 (s, 1H), 6.58 (t, 1H), 6.56 (dm, 1H), 6.44 (dm, 1H), 6.29 (s, 1H), 5.94 (m, 2H), 4.45 (t, 1H), 3.64 (s, 3H), 3.21 (m, 2H), 2.83/2.38 (dd+dd, 2H), 2.45-1.26 (m, 8H), 2.08 (m, 1H), 1.53 (m, 1H), 1.33/1.03 (m+m, 2H), 0.84 (d, 3H). HRMS calculated for C₂₇H₃₂NO₅Cl: 485.1969; found: 486.2041 (M+H).

The diastereoisomer eluting later was collected as Example 839F. ¹H NMR (500 MHz, DMSO-d₆) δ ppm: 7.06 (t, 1H), 6.88 (s, 1H), 6.77 (s, 1H), 6.58 (t, 1H), 6.55 (dm, 1H), 6.45 (dm, 1H), 6.27 (s, 1H), 5.94 (s, 2H), 4.39 (t, 1H), 3.64 (s, 3H), 3.40/3.18 (m+m, 2H), 2.86/2.42 (dd+dd, 2H), 2.44-1.22 (m, 8H), 2.07 (m, 1H), 1.57 (m, 1H), 1.41/0.99 (m+m, 2H), 0.90 (d, 3H). HRMS calculated for C₂₇H₃₂NO₅Cl: 485.1969; found: 486.2043 (M+H).

### Example 839 methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

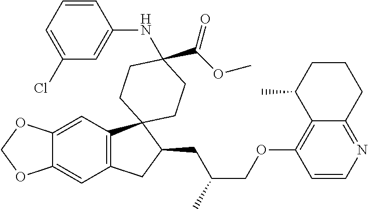 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Using General procedure 30a and Example 839E as the appropriate indane and Preparation 2a1 as the appropriate alcohol, Example 839 was obtained. HRMS calculated for C₃₇H₄₃N₂O₅Cl: 630.2861; found: 631.2917 (M+H).

### Example 840 (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

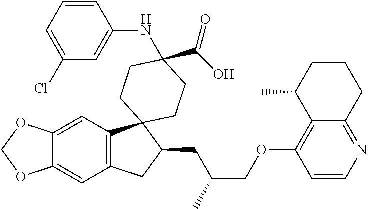 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCO3]

Using General procedure 33a and Example 839 as the appropriate ester, Example 840 was obtained. ¹H NMR (500 MHz, DMSO-d₆) δ ppm: 12.71 (br s, 1H), 8.14 (d, 1H), 7.02 (t, 1H), 6.86 (s, 1H), 6.77 (d, 1H), 6.76 (s, 1H), 6.61 (t, 1H), 6.53 (dd, 1H), 6.51 (dd, 1H), 6.28 (br s, 1H), 5.95/5.93 (s+s, 2H), 3.89/3.84 (dd+dd, 2H), 3.05 (m, 1H), 2.90/2.43 (dd+dd, 2H), 2.76/2.65 (m+m, 2H), 2.38-1.37 (m, 8H), 2.15 (m, 1H), 1.97 (m, 1H), 1.79/1.73 (m+m, 2H), 1.68/1.61 (m+m, 2H), 1.47/1.31 (m+m, 2H), 1.05 (d, 3H), 1.04 (d, 3H). HRMS calculated for C₃₆H₄₁N₂O₅Cl: 616.2704; found: 617.2769 (M+H).

### Preparation of the Sodium Salt of Example 840

The compound of Example 840 (1.68 g) and sodium hydroxide (0.11 g) were suspended in water (450 mL) at 25° C. tBuOH (250 mL) was added to the suspension and the reaction mixture was heated at 60° C. for at least 1 hour (until complete dissolution). The solution was then cooled to −20° C. for fast solidification before the lyophilization step for 96 hours. After isolation from the lyophilization vessel, 1.64 g of the amorphous sodium salt of Example 840 (water content: 7.2%) were obtained. IR (cm⁻¹): 3000-2850; 1575; 1475; 1595; 1498; 1446; 1386; 1290; 1247; 1038; 950-750.

### Example 841 (1r,4R,6′R)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

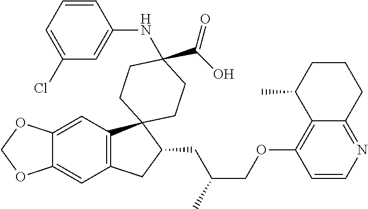 [SMILES: C[C@@H](COc1ccnc2c1[C@H](C)CCC2)C[C@@H]1Cc2cc3c(cc2[C@]12CC[C@@](Nc1cccc(Cl)c1)(C(=O)O)CC2)OCO3]

Using General procedure 32 and Example 839F as the appropriate indane and Preparation 2a1 as the appropriate alcohol, Example 841 was obtained. ¹H NMR (500 MHz, DMSO-d₆) δ ppm: 12.66 (br s, 1H), 8.13 (d, 1H), 7.03 (t, 1H), 6.88 (s, 1H), 6.78 (d, 1H), 6.77 (s, 1H), 6.59 (t, 1H), 6.54 (dm, 1H), 6.48 (dm, 1H), 6.12 (br s, 1H), 5.96-5.91 (m, 2H), 4.00/3.86 (dd+dd, 2H), 3.01 (m, 1H), 2.90/2.48 (dd+dd, 2H), 2.73/2.60 (m+m, 2H), 2.42-1.22 (m, 12H), 2.08 (m, 1H), 2.04 (m, 1H), 1.67/1.23 (m+m, 2H), 1.09 (d, 3H), 1.09 (d, 3H). HRMS calculated for C₃₆H₄₁N₂O₅Cl: 616.2704; found: 617.2770 (M+H).

### Example 842 (1r,4S,6′S)-4-(3-chloroanilino)-6′-[(2R)-2-methyl-3-{[(5S)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

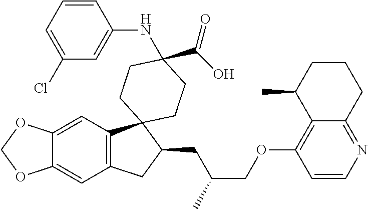 [SMILES: C[C@@H](COc1ccnc2c1[C@@H](C)CCC2)C[C@H]1Cc2cc3c(cc2[C@]12CC[C@@](Nc1cccc(Cl)c1)(C(=O)O)CC2)OCO3]

Using General procedure 32 and Example 839E as the appropriate indane and Preparation 2a2 as the appropriate alcohol, Example 842 was obtained. HRMS calculated for C₃₆H₄₁N₂O₅Cl: 616.2704; found: 617.2767 (M+H).

### Example 843 (1r,4R,6′R)-4-(3-chloroanilino)-6′-[(2R)-2-methyl-3-{[(5S)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

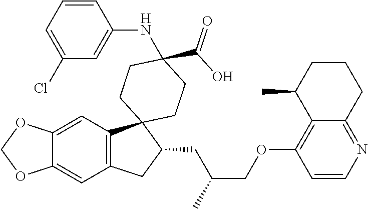 [SMILES: C[C@@H](COc1ccnc2c1[C@@H](C)CCC2)C[C@@H]1Cc2cc3c(cc2[C@]12CC[C@@](Nc1cccc(Cl)c1)(C(=O)O)CC2)OCO3]

Using General procedure 32 and Example 839F as the appropriate indane and Preparation 2a2 as the appropriate alcohol, Example 843 was obtained. ¹H NMR (500 MHz, DMSO-d₆) δ ppm: 12.67 (br s, 1H), 8.14 (s, 1H), 7.02 (t, 1H), 6.90 (s, 1H), 6.78 (s, 1H), 6.78 (d, 1H), 6.59 (t, 1H), 6.53 (dm, 1H), 6.48 (dm, 1H), 6.13 (br s, 1H), 5.96/5.93 (d+d, 2H), 3.92/3.89 (dd+dd, 2H), 3.06 (m, 1H), 2.92/2.49 (dd+dd, 2H), 2.75/2.64 (m+m, 2H), 2.46-1.16 (m, 14H), 2.13 (m, 1H), 2.03 (m, 1H), 1.09 (d, 3H), 1.08 (d, 3H). HRMS calculated for C₃₆H₄₁N₂O₅Cl: 616.2704; found: 617.2781 (M+H).

### Example 844

### Example 844A 3-chloro(2H₄)aniline

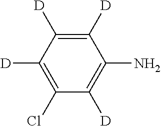 [SMILES: [2H]c1c([2H])c(N)c([2H])c(Cl)c1[2H]]

An oven-dried microwave reactor vial equipped with magnetic stirring bar was charged with K₂(PtCl₄) (141 mg, 0.38 mmol), D₂O (15 mL, 832 mmol), DCl (35 wt % in D₂O, 3.5 mL, 37.6 mmol) and 3-chloroaniline (2.40 g, 18.8 mmol). The headspaces of the vials were flushed with N₂. The mixture was heated to 200° C. for 2 h while stirring at 600 rpm. The mixture was transferred to an oven dried flask and the solvents were removed under reduced pressure. The residue was taken up in D₂O (15 mL, 832 mmol) and DCl (35 wt % in D₂O, 1.77 mL, 18.8 mmol), transferred to an oven-dried microwave reactor vial equipped with magnetic stirring bar and heated to 200° C. for 2 h while stirring at 600 rpm. The mixture was transferred to an oven dried flask and the solvents were removed under reduced pressure. The residue was taken up in D₂O (15 mL, 832 mmol) and DCl (35 wt % in D₂O, 1.78 mL, 18.8 mmol), transferred to an oven-dried microwave reactor vial equipped with magnetic stirring bar and heated to 200° C. for 2 h while stirring at 600 rpm. Then 2 M aq. NaOH solution (50 mL) and DCM (40 mL) were added and the obtained slurry was filtered through a pad of celite. Then the filtrate was extracted with DCM. The combined organic phases were washed with water, then dried over Na₂SO₄, filtered, and the filtrate was concentrated under reduced pressure to obtain Example 844A. According to the ¹H NMR the overall ²H rate is more than 98%. ¹H NMR (500 MHz, DMSO-d₆) δ ppm: 5.37 (s, 2H). HRMS calculated for C₆H₂[²H]₄NCl: 131.0440; found: 132.0516 (M+H).

### Example 844B 1-chloro-3-iodo(2H₄)benzene

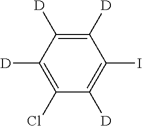 [SMILES: [2H]c1c([2H])c(Cl)c([2H])c(I)c1[2H]]

A 100 mL 3-necked flask fitted with an overhead mechanical stirrer and a dropping funnel was charged with Example 844A (12.20 g, 92.7 mmol), water (35.3 mL, 1955 mmol) and HBF₄ (50% aq. solution, 23.6 mL, 185.4 mmol). The mixture was cooled to 0° C. and stirred vigorously while a solution of NaNO₂ (6.72 g, 97.3 mmol) in water (18.5 mL) was added dropwise during a 15 min period. The reaction mixture was stirred at 0° C. for further 30 min, then the formed precipitate was filtered, washed with ice-cold water (2×5 mL) and Et₂O (3×5 mL) to afford the corresponding diazonium-tetrafluoroborate salt. The obtained intermediate was suspended in water (278 mL) and stirred at 25° C. while a solution of KI (23.08 g, 139 mmol) in water (185 mL) was added dropwise during a 10 min period. The reaction mixture was stirred at 25° C. for further 10 min, until the bubbling ceased. The mixture was extracted with Et₂O, and the combined organic layers were washed with sat. aq. Na₂S₂O₃ solution, dried over Na₂SO₄, and filtered through a short pad of silicagel. The filtrate was concentered under reduced pressure. The crude product was distilled at 140° C./7-9 mbar to obtain Example 844B. ¹H NMR (500 MHz, DMSO-d₆) δ ppm: −. ¹³C NMR (125 MHz, DMSO-d₆) δ ppm: 136.4, 136.0, 131.9, 128.1, 134.3, 95.6.

### Example 844C 6′-bromo-4-{[3-chloro(2H₄)phenyl]amino}-2′H-spiro[cyclohexane-1,5′-indeno[5,6-d][1,3]dioxole]-4-carboxylic acid

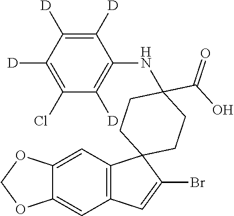 [SMILES: [2H]c1c([2H])c(Cl)c([2H])c(NC2(C(=O)O)CCC3(CC2)C(Br)=Cc2cc4c(cc23)OCO4)c1[2H]]

Using General procedure 16 and Preparation 5b as the appropriate amino acid and Example 844B as the appropriate iodobenzene, a mixture of diastereoisomers was obtained. The diastereoisomers were separated by flash chromatography using heptane and EtOAC as eluents. The diastereoisomer eluting earlier was collected as Example 844C. ¹H NMR (500 MHz, DMSO-d₆) δ ppm: 12.85 (br s, 1H), 7.25 (s, 1H), 6.97 (s, 1H), 6.89 (s, 1H), 6.38 (br s, 1H), 6.02 (s, 2H), 2.34/2.23 (t+d, 4H), 2.15/0.97 (t+d, 4H). HRMS calculated for C₂₂H₁₅[²H]₄NO₄ClBr: 479.0437; found: 480.0510 (M+H).

### Example 844D methyl (1s,4s)-6′-bromo-4-{[3-chloro(2H₄)phenyl]amino}-2′H-spiro[cyclohexane-1,5′-indeno[5,6-d][1,3]dioxole]-4-carboxylate

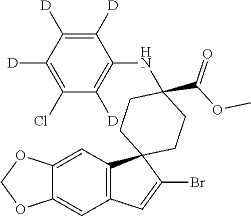 [SMILES: [2H]c1c([2H])c(Cl)c([2H])c(N[C@]2(C(=O)OC)CC[C@@]3(CC2)C(Br)=Cc2cc4c(cc23)OCO4)c1[2H]]

Using General procedure 17b and Example 844C as the appropriate amino acid, Example 844D was obtained. ¹H NMR (500 MHz, DMSO-d₆) δ ppm: 7.27 (s, 1H), 6.97 (s, 1H), 6.89 (s, 1H), 6.47 (s, 1H), 6.02 (s, 2H), 3.68 (s, 3H), 2.34 (td, 2H), 2.25 (d, 2H), 2.16 (td, 2H), 0.97 (d, 2H). HRMS calculated for C₂₃H₁₇[²H]₄NO₄ClBr: 493.0594; found: 494.0666 (M+H).

### Example 844E methyl (1r,4R)-4-{[3-chloro(2H₄)phenyl]amino}-6′-{(2R)-3-[(4-methoxyphenyl)methoxy]-2-methylpropyl}-2′H-spiro[cyclohexane-1,5′-indeno[5,6-d][1,3]dioxole]-4-carboxylate

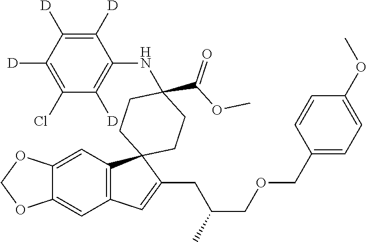 [SMILES: [2H]c1c([2H])c(Cl)c([2H])c(N[C@]2(C(=O)OC)CC[C@@]3(CC2)C(C[C@@H](C)COCc2ccc(OC)cc2)=Cc2cc4c(cc23)OCO4)c1[2H]]

Using General procedure 27b and Example 844D as the appropriate 2-bromo-indene derivative and Preparation 3d as the appropriate Zn reagent, Example 844E was obtained. ¹H NMR (500 MHz, DMSO-d₆) δ ppm: 7.21 (dm, 2H), 7.20 (s, 1H), 6.87 (s, 1H), 6.86 (dm, 2H), 6.40 (s, 1H), 6.31 (s, 1H), 5.97 (s, 2H), 4.39/4.35 (d+d, 2H), 3.73 (s, 3H), 3.68 (s, 3H), 3.32/3.26 (dd+dd, 2H), 2.40-0.80 (m, 8H), 2.29/1.93 (dm+dd, 2H), 2.11 (m, 1H), 0.92 (d, 3H). HRMS calculated for C₃₅H₃₄[²H]₄NO₆Cl: 607.2639; found: 608.2712 (M+H).

### Example 844F methyl (1r,4R)-4-{[3-chloro(2H₄)phenyl]amino}-6′-{(2R)-3-[(4-methoxyphenyl)methoxy]-2-methylpropyl}-6′,7′-dihydro-2′H-spiro[cyclohexane-1,5′-indeno[5,6-d][1,3]dioxole]-4-carboxylate

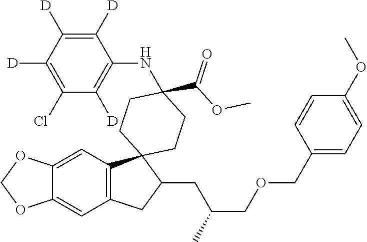

Using General procedure 19 and Example 844E as the appropriate indene, Example 844F was obtain as a mixture of diastereoisomers. ¹H NMR (500 MHz, DMSO-d₆) δ ppm: 7.20 (m, 2H), 6.88/6.87 (s, 1H), 6.86/6.80 (m, 2H), 6.75/6.74 (s, 1H), 6.30/6.28 (s, 1H), 5.96-5.92 (d+d, 2H), 4.43-4.30 (d+d, 2H), 3.72/3.69 (s, 3H), 3.64 (s, 3H), 3.37-3.09 (m, 2H), 2.82/2.40 (dd+dd, 2H), 2.47-0.95 (m, 11H), 2.06 (m, 1H), 0.93/0.88 (d, 3H). HRMS calculated for C₃₅H₃₆[²H]₄NO₆Cl: 609.2795; found: 610.2838 and 610.2840 (M+H).

### Example 844G methyl (1r,4S,6′S)-4-{[3-chloro(2H₄)phenyl]amino}-6′-[(2R)-3-hydroxy-2-methylpropyl]-6′,7′-dihydro-2′H-spiro[cyclohexane-1,5′-indeno[5,6-d][1,3]dioxole]-4-carboxylate

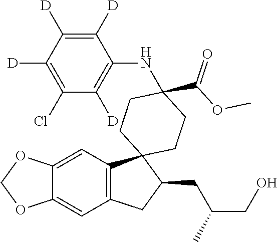 [SMILES: [2H]c1c([2H])c(Cl)c([2H])c(N[C@]2(C(=O)OC)CC[C@]3(CC2)c2cc4c(cc2C[C@@H]3C[C@@H](C)CO)OCO4)c1[2H]]

Using General procedure 28b and Example 844F as the appropriate PMB derivative, a mixture of diastereoisomers was obtained. The diastereoisomers were separated by chiral chromatography. Column: AD, 100×500 mm, 20 μm, Eluents: iPrOH/heptane. The diastereoisomer eluting earlier was collected as Example 844G. ¹H NMR (500 MHz, DMSO-d₆) δ ppm: 6.87 (s, 1H), 6.76 (s, 1H), 6.28 (s, 1H), 5.95/5.93 (d+d, 2H), 4.44 (t, 1H), 3.64 (s, 3H), 3.21 (t, 2H), 2.83/2.39 (dd+dd, 2H), 2.43-1.29 (m, 8H), 2.07 (m, 1H), 1.53 (m, 1H), 1.32/1.03 (t+t, 2H), 0.84 (d, 3H). HRMS calculated for C₂₇H₂₈[²H]₄NO₅Cl: 489.2220; found: 490.2275 (M+H).

### Example 844 (1r,4S,6′S)-4-{[3-chloro(2H₄)phenyl]amino}-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

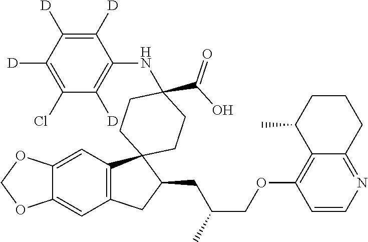 [SMILES: [2H]c1c([2H])c(Cl)c([2H])c(N[C@]2(C(=O)O)CC[C@]3(CC2)c2cc4c(cc2C[C@@H]3C[C@@H](C)COc2ccnc3c2[C@H](C)CCC3)OCO4)c1[2H]]

Using General procedure 32 and Example 844G as the appropriate indane and Preparation 2a1 as the appropriate alcohol, Example 844 was obtained. HRMS calculated for C₃₆H₃₇[²H]₄N₂O₅Cl: 620.2955; found: 621.3030 (M+H).

### Example 845

### Example 845A (1s,4s)-6′-bromo-4-(3-chloro-4-fluoroanilino)-2′H-spiro[cyclohexane-1,5′-indeno[5,6-d][1,3]dioxole]-4-carboxylic acid

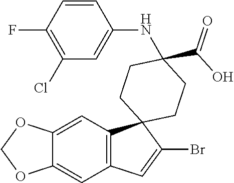 [SMILES: O=C(O)[C@]1(Nc2ccc(F)c(Cl)c2)CC[C@]2(CC1)C(Br)=Cc1cc3c(cc12)OCO3]

Using General procedure 16 and Preparation 5b as the appropriate amino acid and 2-chloro-1-fluoro-4-iodo-benzene as the appropriate iodobenzene, a mixture of diastereoisomers was obtained. The diastereoisomers were separated by flash chromatography using heptane and EtOAC as eluents. The diastereoisomer eluting later was collected as Example 845A. ¹H NMR (500 MHz, DMSO-d₆) δ ppm: 12.86 (br s, 1H), 7.25 (s, 1H), 7.14 (t, 1H), 6.97 (s, 1H), 6.88 (s, 1H), 6.67 (dd, 1H), 6.55 (m, 1H), 6.28 (br s, 1H), 6.02 (s, 2H), 2.33/2.21 (m+m, 4H), 2.14/0.96 (m+m, 4H).

### Example 845B methyl (1s,4s)-6′-bromo-4-(3-chloro-4-fluoroanilino)-2′H-spiro[cyclohexane-1,5′-indeno[5,6-d][1,3]dioxole]-4-carboxylate

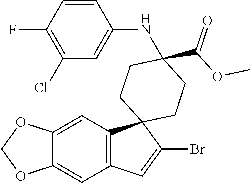 [SMILES: COC(=O)[C@]1(Nc2ccc(F)c(Cl)c2)CC[C@]2(CC1)C(Br)=Cc1cc3c(cc12)OCO3]

Using General procedure 17b and Example 845A as the appropriate amino acid, Example 845B was obtained. ¹H NMR (500 MHz, DMSO-d₆) δ ppm: 7.28 (s, 1H), 7.14 (t, 1H), 6.97 (s, 1H), 6.89 (s, 1H), 6.68 (dd, 1H), 6.49 (m, 1H), 6.35 (s, 1H), 6.02 (s, 2H), 3.68 (s, 3H), 2.34/2.23 (m+m, 4H), 2.15/0.97 (m+m, 4H).

### Example 845C methyl (1r,4R)-4-(3-chloro-4-fluoroanilino)-6′-{(2R)-3-[(4-methoxyphenyl)methoxy]-2-methylpropyl}-2′H-spiro[cyclohexane-1,5′-indeno[5,6-d][1,3]dioxole]-4-carboxylate

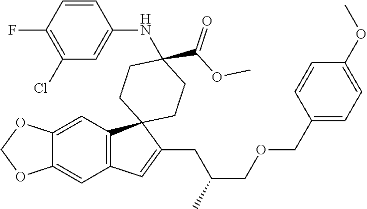 [SMILES: COc1ccc(COC[C@H](C)CC2=Cc3cc4c(cc3[C@]23CC[C@@](Nc2ccc(F)c(Cl)c2)(C(=O)OC)CC3)OCO4)cc1]

Using General procedure 27b and Example 845B as the appropriate 2-bromo-indene derivative and Preparation 3d as the appropriate Zn reagent, Example 845C was obtained. ¹H NMR (500 MHz, DMSO-d₆) δ ppm: 7.21 (m, 2H), 7.20 (s, 1H), 7.12 (t, 1H), 6.87 (s, 1H), 6.85 (m, 2H), 6.70 (dd, 1H), 6.48 (m, 1H), 6.31 (s, 1H), 6.27 (s, 1H), 5.97 (s, 2H), 4.38/4.05 (d+d, 2H), 3.72 (s, 3H), 3.68 (s, 3H), 3.32/3.26 (dd+dd, 2H), 2.33/2.16 (m+m, 4H), 2.30/1.93 (m+m, 2H), 2.11 (m, 1H), 2.05/0.84 (m+m, 4H), 0.92 (d, 3H). HRMS calculated for C₃₅H₃₇NO₆FCl: 621.2294; found: 622.2366 (M+H).

### Example 845D methyl (1r,4R)-4-(3-chloro-4-fluoroanilino)-6′-{(2R)-3-[(4-methoxyphenyl)methoxy]-2-methylpropyl}-6′,7′-dihydro-2′H-spiro[cyclohexane-1,5′-indeno[5,6-d][1,3]dioxole]-4-carboxylate

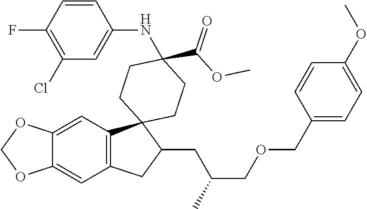

Using General procedure 19 and Example 845C as the appropriate indene, Example 845D was obtain as a mixture of diastereoisomers. ¹H NMR (500 MHz, DMSO-d₆) δ ppm: 8.60/6.86 (m, 2H), 7.20 (m, 2H), 7.10 (t, 1H), 6.87/6.86 (s, 1H), 6.75 (s, 1H), 6.68/6.66 (dd, 1H), 6.47 (m, 1H), 6.17/6.15 (s, 1H), 5.95/5.93 (d+d, 2H), 4.41-4.30 (d+d, 2H), 3.72/3.69 (s, 3H), 3.65 (s, 3H), 3.35-3.10 (m, 2H), 2.83/2.40 (dd+dd, 2H), 2.46-0.96 (m, 10H), 2.06 (m, 1H), 1.74 (m, 1H), 0.93/0.88 (d, 3H). HRMS calculated for C₃₅H₃₉NO₆FCl: 623.2450; found: 624.2517 and 624.2525 (M+H).

### Example 845E methyl (1r,4S,6′S)-4-(3-chloro-4-fluoroanilino)-6′-[(2R)-3-hydroxy-2-methylpropyl]-6′,7′-dihydro-2′H-spiro[cyclohexane-1,5′-indeno[5,6-d][1,3]dioxole]-4-carboxylate

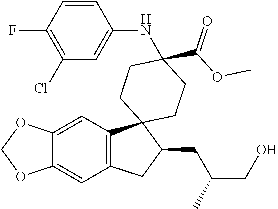 [SMILES: COC(=O)[C@]1(Nc2ccc(F)c(Cl)c2)CC[C@@]2(CC1)c1cc3c(cc1C[C@@H]2C[C@@H](C)CO)OCO3]

Using General procedure 28b and Example 845D as the appropriate PMB derivative, a mixture of diastereoisomers was obtained. The diastereoisomers were separated by chiral chromatography. Column: AD, 50×500 mm, 20 μm, Eluents: iPrOH/heptane. The diastereoisomer eluting earlier was collected as Example 845E. ¹H NMR (500 MHz, DMSO-d₆) δ ppm: 7.11 (t, 1H), 6.87 (s, 1H), 6.76 (s, 1H), 6.66 (dd, 1H), 6.46 (dt, 1H), 6.15 (s, 1H), 5.95/5.93 (d+d, 2H), 4.44 (t, 1H), 3.65 (s, 3H), 3.21 (m, 2H), 2.44-1.26 (m, 8H), 2.83/2.38 (dd+dd, 2H), 2.08 (m, 1H), 1.53 (m, 1H), 1.32/1.02 (td+m, 2H), 0.84 (d, 3H). HRMS calculated for C₂₇H₃₁NO₅FCl: 503.1875; found: 504.1946 (M+H).

### Example 845 (1r,4S,6′S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

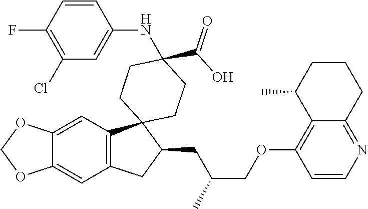 [SMILES: C[C@@H](COc1ccnc2c1[C@H](C)CCC2)C[C@H]1Cc2cc3c(cc2[C@]12CC[C@@](Nc1ccc(F)c(Cl)c1)(C(=O)O)CC2)OCO3]

Using General procedure 32 and Example 845E as the appropriate indane and Preparation 2a1 as the appropriate alcohol, Example 845 was obtained. HRMS calculated for C₃₆H₄₀N₂05FCl: 634.2610; found: 635.2694 (M+H).

### Example 846

### Example 846A (1s,4s)-6′-bromo-4-(3-chloro-2-fluoroanilino)-2′H-spiro[cyclohexane-1,5′-indeno[5,6-d][1,3]dioxole]-4-carboxylic acid

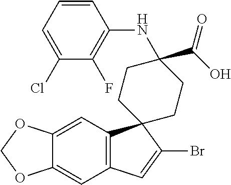 [SMILES: O=C(O)[C@]1(Nc2cccc(Cl)c2F)CC[C@]2(CC1)C(Br)=Cc1cc3c(cc12)OCO3]

Using General procedure 16 and Preparation 5b as the appropriate amino acid and 1-chloro-2-fluoro-3-iodo-benzene as the appropriate iodobenzene, a mixture of diastereoisomers was obtained. The diastereoisomers were separated by flash chromatography using heptane and EtOAC as eluents. The diastereoisomer eluting later was collected as Example 846A. ¹H NMR (500 MHz, DMSO-d₆) δ ppm: 12.94 (br s, 1H), 7.26 (s, 1H), 6.98 (s, 1H), 6.97 (m, 1H), 6.89 (s, 1H), 6.76 (m, 1H), 6.53 (m, 1H), 6.03 (s, 2H), 5.86 (s, 1H), 2.41-2.28 (m, 4H), 2.12/0.95 (m+m, 4H). HRMS calculated for C₂₂H₁₈NO₄FClBr: 493.0092; found: 494.0166 (M+H).

### Example 846B methyl (1s,4s)-6′-bromo-4-(3-chloro-2-fluoroanilino)-2′H-spiro[cyclohexane-1,5′-indeno[5,6-d][1,3]dioxole]-4-carboxylate

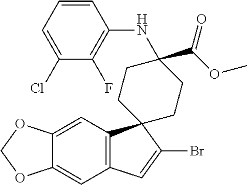 [SMILES: COC(=O)[C@]1(Nc2cccc(Cl)c2F)CC[C@]2(CC1)C(Br)=Cc1cc3c(cc12)OCO3]

Using General procedure 17b and Example 846A as the appropriate amino acid, Example 846B was obtained. ¹H NMR (500 MHz, DMSO-d₆) δ ppm: 7.27 (s, 1H), 6.98 (s, 1H), 6.96 (m, 1H), 6.90 (s, 1H), 6.78 (m, 1H), 6.46 (m, 1H), 6.03 (s, 2H), 6.01 (d, 1H), 3.71 (s, 3H), 2.41-2.29 (m, 4H), 2.12/0.96 (m+m, 4H). HRMS calculated for C₂₃H₂₀NO₄FClBr: 507.0248; found: 508.0319 (M+H).

### Example 846C methyl (1r,4R)-4-(3-chloro-2-fluoroanilino)-6′-{(2R)-3-[(4-methoxyphenyl)methoxy]-2-methylpropyl}-2′H-spiro[cyclohexane-1,5′-indeno[5,6-d][1,3]dioxole]-4-carboxylate

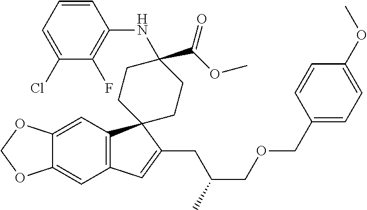 [SMILES: COc1ccc(COC[C@H](C)CC2=Cc3cc4c(cc3[C@]23CC[C@@](Nc2cccc(Cl)c2F)(C(=O)OC)CC3)OCO4)cc1]

Using General procedure 27b and Example 846B as the appropriate 2-bromo-indene derivative and Preparation 3d as the appropriate Zn reagent, Example 846C was obtained. ¹H NMR (500 MHz, DMSO-d₆) δ ppm: 7.21 (s, 1H), 7.20 (m, 2H), 6.98 (s, 2H), 6.96 (m, 1H), 6.87 (s, 1H), 6.85 (m, 2H), 6.79 (m, 1H), 6.48 (m, 1H), 6.31 (s, 1H), 5.95 (d, 1H), 4.38/4.35 (d+d, 2H), 3.72 (s, 3H), 3.71 (s, 3H), 3.31/3.25 (dd+dd, 2H), 2.34/2.26 (m+m, 4H), 2.29/1.92 (m+m, 2H), 2.09 (m, 1H), 2.02/0.84 (m+m, 4H), 0.92 (d, 3H). HRMS calculated for C₃₅H₃₇NO₆FCl: 621.2294; found: 622.2273 (M+H).

### Example 846D methyl (1r,4R)-4-(3-chloro-2-fluoroanilino)-6′-{(2R)-3-[(4-methoxyphenyl)methoxy]-2-methylpropyl}-6′,7′-dihydro-2′H-spiro[cyclohexane-1,5′-indeno[5,6-d][1,3]dioxole]-4-carboxylate

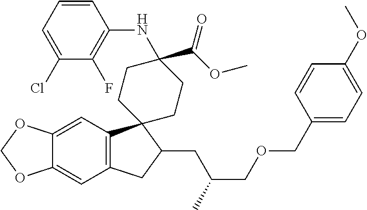

Using General procedure 19 and Example 846C as the appropriate indene, Example 846D was obtain as a mixture of diastereoisomers. ¹H NMR (500 MHz, DMSO-d₆) δ ppm: 7.20/7.19 (m, 2H), 6.94 (m, 1H), 6.88/6.87 (s, 1H), 6.86/6.80 (m, 2H), 6.76 (m, 1H), 6.75 (s, 1H), 6.49/6.47 (m, 1H), 5.95/5.93 (d+d, 2H), 5.79/5.76 (d, 1H), 4.42-4.30 (d+d, 2H), 3.72/3.69 (s, 3H), 3.66 (s, 3H), 3.35-3.17 (m, 2H), 2.82/2.39 (dd+dd, 2H), 2.46-0.96 (m, 11H), 2.06 (m, 1H), 0.93/0.88 (d, 3H). HRMS calculated for C₃₅H₃₉NO₆FCl: 623.2450; found: 624.2477 and 624.2480 (M+H).

### Example 846E methyl (1r,4S,6′S)-4-(3-chloro-2-fluoroanilino)-6′-[(2R)-3-hydroxy-2-methylpropyl]-6′,7′-dihydro-2′H-spiro[cyclohexane-1,5′-indeno[5,6-d][1,3]dioxole]-4-carboxylate

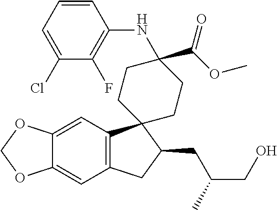 [SMILES: COC(=O)[C@]1(Nc2cccc(Cl)c2F)CC[C@@]2(CC1)c1cc3c(cc1C[C@@H]2C[C@@H](C)CO)OCO3]

Using General procedure 28b and Example 846D as the appropriate PMB derivative, a mixture of diastereoisomers was obtained. The diastereoisomers were separated by flash chromatography using heptane and EtOAc as solvents. The diastereoisomer eluting later was collected as Example 846E. ¹H NMR (500 MHz, DMSO-d₆) δ ppm: 6.94 (m, 1H), 6.88 (s, 1H), 6.76 (m, 1H), 6.76 (s, 1H), 6.48 (m, 1H), 5.95/5.93 (d, 2H), 5.79 (d, 1H), 4.44 (t, 1H), 3.66 (s, 3H), 3.20 (m, 2H), 2.83/2.38 (dd+dd, 2H), 2.46-1.26 (m, 8H), 2.07 (m, 1H), 1.53 (m, 1H), 1.33/1.01 (m+m, 2H), 0.84 (d, 3H). HRMS calculated for C₂₇H₃₁NO₅FCl: 503.1875; found: 504.1950 (M+H).

### Example 846 (1r,4S,6′S)-4-(3-chloro-2-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

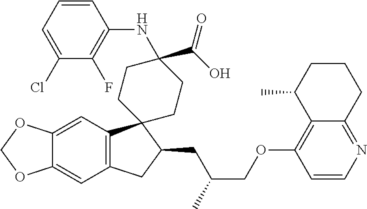 [SMILES: C[C@@H](COc1ccnc2c1[C@H](C)CCC2)C[C@H]1Cc2cc3c(cc2[C@]12CC[C@@](Nc1cccc(Cl)c1F)(C(=O)O)CC2)OCO3]

Using General procedure 32 and Example 846E as the appropriate indane and Preparation 2a1 as the appropriate alcohol, Example 846 was obtained. ¹H NMR (500 MHz, DMSO-d₆) δ ppm: 12.8 (br s, 1H), 8.14 (d, 1H), 6.93 (td, 1H), 6.87 (s, 1H), 6.77 (d, 1H), 6.77 (s, 1H), 6.72 (td, 1H), 6.57 (td, 1H), 5.95/5.94 (d+d, 2H), 5.62 (br s, 1H), 3.87 (m, 2H), 3.04 (m, 1H), 2.89/2.42 (dd+dd, 2H), 2.76/2.65 (dm+m, 2H), 2.44-1.24 (m, 14H), 2.14 (m, 1H), 1.97 (m, 1H), 1.03 (d, 3H), 1.03 (d, 3H). HRMS calculated for C₃₆H₄₀N₂05FCl: 634.2610; found: 635.2690 (M+H).

### Example 847

### Example 847A (1s,4s)-6′-bromo-4-(3-chloro-2-methylanilino)-2′H-spiro[cyclohexane-1,5′-indeno[5,6-d][1,3]dioxole]-4-carboxylic acid

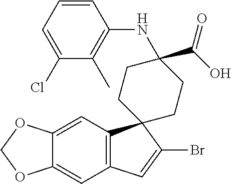 [SMILES: Cc1c(Cl)cccc1N[C@]1(C(=O)O)CC[C@@]2(CC1)C(Br)=Cc1cc3c(cc12)OCO3]

Using General procedure 16 and Preparation 5b as the appropriate amino acid and 1-chloro-3-iodo-2-methyl-benzene as the appropriate iodobenzene, a mixture of diastereoisomers was obtained. The diastereoisomers were separated by flash chromatography using heptane and EtOAC as eluents. The diastereoisomer eluting later was collected as Example 847A. ¹H NMR (500 MHz, DMSO-d₆) δ ppm: 12.87 (br s, 1H), 7.27 (s, 1H), 6.99 (t, 1H), 6.98 (s, 1H), 6.90 (s, 1H), 6.74 (d, 1H), 6.34 (d, 1H), 6.03 (s, 2H), 5.15 (br s, 1H), 2.40 (s, 3H), 2.39-2.26 (m, 4H), 2.11/0.91 (m+m, 4H). HRMS calculated for C₂₃H₂₁NO₄ClBr: 489.0342; found: 490.0417 (M+H).

### Example 847B methyl (1s,4s)-6′-bromo-4-(3-chloro-2-methylanilino)-2′H-spiro[cyclohexane-1,5′-indeno[5,6-d][1,3]dioxole]-4-carboxylate

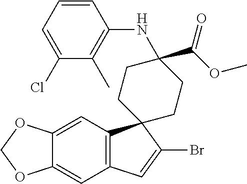 [SMILES: COC(=O)[C@]1(Nc2cccc(Cl)c2C)CC[C@]2(CC1)C(Br)=Cc1cc3c(cc12)OCO3]

Using General procedure 17b and Example 847A as the appropriate amino acid, Example 847B was obtained. ¹H NMR (500 MHz, DMSO-d₆) δ ppm: 7.28 (s, 1H), 6.99 (t, 1H), 6.98 (s, 1H), 6.90 (s, 1H), 6.76 (d, 1H), 6.22 (d, 1H), 6.03 (s, 2H), 5.29 (s, 1H), 3.73 (s, 3H), 2.41 (s, 3H), 2.39-2.27 (m, 4H), 2.12/0.92 (td+d, 4H). HRMS calculated for C₂₄H₂₃NO₄ClBr: 503.0499; found: 504.0573 (M+H).

### Example 847C methyl (1r,4R)-4-(3-chloro-2-methylanilino)-6′-{(2R)-3-[(4-methoxyphenyl)methoxy]-2-methylpropyl}-2′H-spiro[cyclohexane-1,5′-indeno[5,6-d][1,3]dioxole]-4-carboxylate

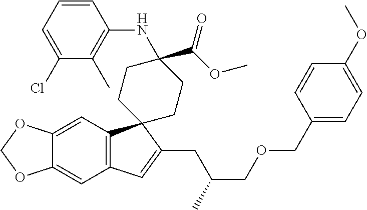 [SMILES: COc1ccc(COC[C@H](C)CC2=Cc3cc4c(cc3[C@]23CC[C@@](Nc2cccc(Cl)c2C)(C(=O)OC)CC3)OCO4)cc1]

Using General procedure 27b and Example 847B as the appropriate 2-bromo-indene derivative and Preparation 3d as the appropriate Zn reagent, Example 847C was obtained. ¹H NMR (500 MHz, DMSO-d₆) δ ppm: 7.21 (s, 1H), 7.19 (m, 2H), 6.99 (t, 1H), 6.87 (s, 1H), 6.86 (m, 2H), 6.76 (d, 1H), 6.31 (m, 1H), 6.21 (d, 1H), 5.98 (s, 2H), 5.25 (s, 1H), 4.37/4.35 (d+d, 2H), 3.73 (s, 3H), 3.72 (s, 3H), 3.29/3.26 (dd+dd, 2H), 2.34/2.28 (m+m, 4H), 2.34 (s, 3H), 2.32/1.93 (m+m, 2H), 2.09 (m, 1H), 2.01/0.80 (m+m, 4H), 0.91 (d, 3H). HRMS calculated for C₃₆H₄₀NO₆Cl: 617.2544; found: 618.2626 (M+H).

### Example 847D methyl (1r,4R)-4-(3-chloro-2-methylanilino)-6′-{(2R)-3-[(4-methoxyphenyl)methoxy]-2-methylpropyl}-6′,7′-dihydro-2′H-spiro[cyclohexane-1,5′-indeno[5,6-d][1,3]dioxole]-4-carboxylate

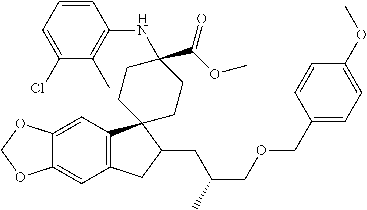

Using General procedure 19 and Example 847C as the appropriate indene, Example 847D was obtain as a mixture of diastereoisomers. ¹H NMR (500 MHz, DMSO-d₆) δ ppm: 7.21/7.17 (s/s, 2H), 6.97/6.96 (t/t, 1H), 6.97/6.94 (s/s, 1H), 6.87/6.80 (s/s, 2H), 6.76/6.75 (s/s, 1H), 6.74/6.73 (d/d, 1H), 6.23/6.21 (d/d, 1H), 5.96/5.93 (m+m, 2H), 5.08/5.06 (s/s, 1H), 4.37/4.36/4.33 (d+d/s, 2H), 3.73/3.70 (s/s, 3H), 3.67 (s, 3H), 3.29/3.23/3.22/3.14 (dd+dd/dd+dd, 2H), 2.85/2.79/2.41/2.37 (dd+dd/dd+dd, 2H), 2.42-1.09 (m, 8H), 2.29/2.27 (s/s, 3H), 1.98/1.96 (m/m, 1H), 1.74/1.72 (m/m, 1H), 1.51/1.39/1.15/1.04 (t+t/t+t, 2H), 0.93/0.86 (d/d, 3H). HRMS calculated for C₃₆H₄₂NO₆Cl: 619.2701; found: 620.2783 and 620.2775 (M+H).

### Example 847E methyl (1r,4S,6′S)-4-(3-chloro-2-methylanilino)-6′-[(2R)-3-hydroxy-2-methylpropyl]-6′,7′-dihydro-2′H-spiro[cyclohexane-1,5′-indeno[5,6-d][1,3]dioxole]-4-carboxylate

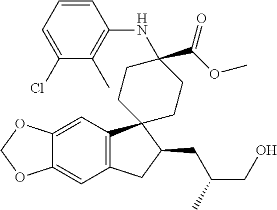 [SMILES: COC(=O)[C@]1(Nc2cccc(Cl)c2C)CC[C@@]2(CC1)c1cc3c(cc1C[C@@H]2C[C@@H](C)CO)OCO3]

Using General procedure 28b and Example 847D as the appropriate PMB derivative, a mixture of diastereoisomers was obtained. The diastereoisomers were separated by chiral chromatography. Column: OD, 100×500 mm, 20 μm, Eluents: iPrOH/heptane. The diastereoisomer eluting earlier was collected as Example 847E. ¹H NMR (500 MHz, DMSO-d₆) δ ppm: 6.96 (s, 1H), 6.96 (t, 1H), 6.77 (s, 1H), 6.73 (d, 1H), 6.22 (d, 1H), 5.96/5.93 (d+d, 2H), 5.08 (s, 1H), 4.46 (t, 1H), 3.67 (s, 3H), 3.21 (m, 2H), 2.80/2.36 (dd+dd, 2H), 2.44-1.09 (m, 8H), 2.29 (s, 3H), 1.98 (m, 1H), 1.52 (m, 1H), 1.40/1.04 (m+m, 2H), 0.83 (d, 3H). HRMS calculated for C₂₈H₃₄NO₅Cl: 499.2126; found: 500.2202 (M+H).

### Example 847 (1r,4S,6′S)-4-(3-chloro-2-methylanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

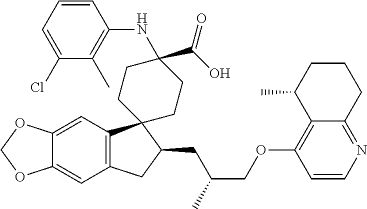 [SMILES: Cc1c(Cl)cccc1N[C@]1(C(=O)O)CC[C@]2(CC1)c1cc3c(cc1C[C@@H]2C[C@@H](C)COc1ccnc2c1[C@H](C)CCC2)OCO3]

Using General procedure 32 and Example 847E as the appropriate indane and Preparation 2a1 as the appropriate alcohol, Example 847 was obtained. ¹H NMR (500 MHz, DMSO-d₆) δ ppm: 12.73 (br s, 1H), 8.15 (d, 1H), 6.96 (s, 1H), 6.95 (t, 1H), 6.79 (d, 1H), 6.78 (s, 1H), 6.69 (d, 1H), 6.35 (d, 1H), 5.96/5.94 (d+d, 2H), 4.95 (br s, 1H), 3.90/3.88 (dd+dd, 2H), 3.05 (m, 1H), 2.86/2.40 (dd+dd, 2H), 2.76/2.66 (m+m, 2H), 2.46-1.10 (m, 12H), 2.22 (s, 3H), 2.05 (m, 1H), 1.96 (m, 1H), 1.55/1.33 (m+m, 2H), 1.04 (d, 3H), 1.03 (d, 3H). HRMS calculated for C₃₇H₄₃N₂O₅Cl: 630.2861; found: 631.2936 (M+H).

### Example 848

### Example 848A methyl (1r,4S,6′S)-4-(3-chloroanilino)-6′-[(2R)-3-hydroxy-2-methylpropyl]-2′,2′-dimethyl-6′,7′-dihydro-2′H-spiro[cyclohexane-1,5′-indeno[5,6-d][1,3]dioxole]-4-carboxylate

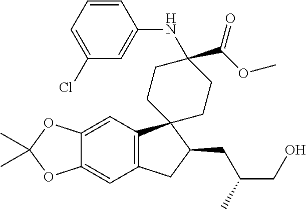 [SMILES: COC(=O)[C@]1(Nc2cccc(Cl)c2)CC[C@@]2(CC1)c1cc3c(cc1C[C@@H]2C[C@@H](C)CO)OC(C)(C)O3]

Example 839E (0.486 g, 1 mmol) was dissolved in DCM (10 mL) and cooled to 0° C. 1 M BBr₃ solution in heptane (3 mL, 3 mmol) was added dropwise and the mixture was stirred at 0° C. for 30 min. Then MeOH was added and the mixture was concentrated under reduced pressure. MeOH was added again and the mixture was concentrated under reduced pressure. The residue was transferred to a microwave tube and then 2,2-dimethoxypropane (8 mL) and [(1S,4R)-7,7-dimethyl-2-oxo-norbornan-1-yl]methanesulfonic acid (0.116 g, 0.5 mmol) were added. The mixture was stirred at 130° C. for 30 min, heated via microwave irradiation. 1.25 M HCl solution in EtOH (0.15 mL) was added and the mixture was concentrated under reduced pressure. The crude product was purified via flash chromatography using heptane and EtOAc as eluents to obtain Example 848A. ¹H NMR (400 MHz, DMSO-d₆) δ ppm: 7.06 (t, 1H), 6.78 (s, 1H), 6.65 (s, 1H), 6.58 (t, 1H), 6.56 (dm, 1H), 6.44 (dm, 1H), 6.28 (s, 1H), 4.44 (br s, 1H), 3.64 (s, 3H), 3.21 (m, 2H), 2.80/2.37 (dd+dd, 2H), 2.46-1.25 (m, 8H), 2.05 (m, 1H), 1.62 (s, 3H), 1.59 (s, 3H), 1.53 (m, 1H), 1.33/1.03 (m+m, 2H), 0.84 (d, 3H). HRMS calculated for C₂₉H₃₆NO₅Cl: 513.2282; found: 514.2359 (M+H).

### Example 848 (1r,4S,6′S)-4-(3-chloroanilino)-2′,2′-dimethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

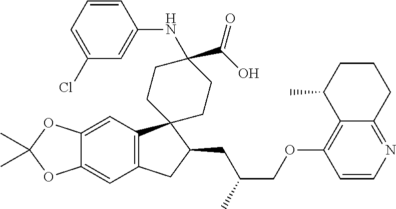 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OC(C)(C)O3]

Using General procedure 32 and Example 848A as the appropriate indane and Preparation 2a1 as the appropriate alcohol, Example 848 was obtained. ¹H NMR (500 MHz, DMSO-d₆) δ ppm: 12.56 (br s, 1H), 8.14 (d, 1H), 7.02 (t, 1H), 6.76 (s, 1H), 6.76 (d, 1H), 6.65 (s, 1H), 6.60 (t, 1H), 6.52 (dm, 1H), 6.51 (dm, 1H), 6.18 (br s, 1H), 3.89/3.85 (dd+dd, 2H), 3.04 (m, 1H), 2.87/2.40 (dd+dd, 2H), 2.76/2.65 (m+m, 2H), 2.45-1.28 (m, 12H), 2.11 (m, 1H), 1.96 (m, 1H), 1.61/1.59 (s, 6H), 1.47/1.30 (m+m, 2H), 1.04 (d, 3H), 1.03 (d, 3H). HRMS calculated for C₃₈H₄₅N₂O₅Cl: 644.3017; found: 645.3074 (M+H).

### Example 849

### Example 849A (2E)-3-(2,2-difluoro-2H-1,3-benzodioxol-5-yl)prop-2-enoic acid

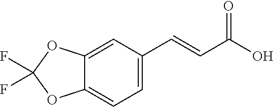 [SMILES: O=C(O)/C=C/c1ccc2c(c1)OC(F)(F)O2]

2,2-difluoro-2H-1,3-benzodioxole-5-carbaldehyde (44.0 g, 138 mmol) was dissolved in pyridine (62 mL), then malonic acid (24.6 g, 236.4 mmol) and piperidine (1.23 mL, 7.3 mmol) were added. The mixture was stirred at reflux temperature for 2 h, then it was cooled. The mixture was poured onto a mixture of 800 mL ice and 80 mL cc. aq. HCl solution. The precipitation was filtered off and dried. The crude product was suspended in 5% aq. NaOH solution, washed with DCM. The aq. phase was acidified with 5% aq. HCl solution and the white precipitation was filtered off, washed with water and dried to obtain Example 849A. ¹H NMR (500 MHz, DMSO-d₆) δ ppm: 12.47 (s, 1H), 7.89 (d, 1H), 7.59 (s, 1H), 7.55 (dd, 1H), 7.45 (d, 1H), 6.57 (d, 1H). HRMS calculated for C₁₀H₆O₄F₂: 228.0234; found: 227.0162 (M−H).

### Example 849B 3-(2,2-difluoro-2H-1,3-benzodioxol-5-yl)propanoic acid

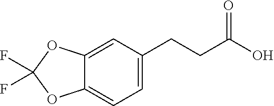 [SMILES: O=C(O)CCc1ccc2c(c1)OC(F)(F)O2]

Example 849A (3.9 g, 17.1 mmol) was dissolved in EtOH (47 mL). 10% Pd/C (0.12 g) was added to the mixture. The flask was evacuated and backfilled with N₂ (×3), then evacuated and filled with H₂. The reaction mixture was stirred at rt overnight. The reaction mixture was filtered, washed with EtOH, and the filtrate was concentrated under reduced pressure to obtain Example 849B. ¹H NMR (500 MHz, DMSO-d₆) δ ppm: 12.17 (s, 1H), 7.31 (d, 1H), 7.29 (d, 1H), 7.06 (dd, 1H), 2.83 (t, 2H), 2.54 (t, 2H). HRMS calculated for C₁₀H₈O₄F₂: 230.0391; found: 229.0318 (M−H).

### Example 849C 2,2-difluoro-6,7-dihydro-2H,5H-indeno[5,6-d][1,3]dioxol-5-one

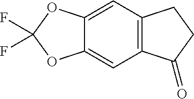 [SMILES: O=C1CCc2cc3c(cc21)OC(F)(F)O3]

Example 849B (3.7 g, 16.1 mmol) was dissolved in dry DCM (90 mL) and DMF (0.16 mL), then oxalyl dichloride (1.65 mL, 19.3 mmol) was added dropwise. The mixture was stirred at rt overnight. TfOH (2.11 mL, 24 mmol) was added to the mixture, then it was stirred for 3 h. The mixture was poured into water (45 mL) and the organic phase was washed with water, sat. aq. NaHCO₃ solution and brine. The organic phase was dried over Na₂SO₄, filtered and the filtrate was concentrated under reduced pressure. The crude product was purified via flash chromatography using hexane and EtOAc as eluents to obtain Example 849C. ¹H NMR (500 MHz, DMSO-d₆) δ ppm: 7.64 (s, 1H), 7.61 (s, 1H), 3.10 (m, 2H), 2.68 (m, 2H). HRMS calculated for C₁₀H₆O₃F₂: 212.0285; found: 212.0297 (M).

### Example 849D 6-bromo-2,2-difluoro-6,7-dihydro-2H,5H-indeno[5,6-d][1,3]dioxol-5-one

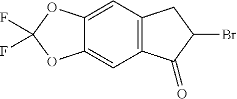 [SMILES: O=C1c2cc3c(cc2CC1Br)OC(F)(F)O3]

Using General procedure 5 and Example 849C as the appropriate indan-1-one, Example 849D was obtained. ¹H NMR (500 MHz, DMSO-d₆) δ ppm: 7.76 (s, 1H), 7.65 (s, 1H), 5.05 (dd, 1H), 3.88/3.31 (dd+dd, 2H). HRMS calculated for C₁₀H₅O₃BrF₂: 289.9390; found: 289.9388 (M).

### Example 849E 6-bromo-2,2-difluoro-6,7-dihydro-2H,5H-indeno[5,6-d][1,3]dioxol-5-ol

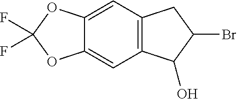 [SMILES: OC1c2cc3c(cc2CC1Br)OC(F)(F)O3]

Using General procedure 6 and Example 849D as the appropriate bromo-indan-1-one, Example 849E was obtained. ¹H NMR (500 MHz, DMSO-d₆) δ ppm: 7.33 (s, 1H), 7.27 (s, 1H), 5.96 (br s, 1H), 4.93 (d, 1H), 4.91 (m, 1H), 3.42/3.18 (dd+dd, 2H). HRMS calculated for C₁₀H₇O₃BrF₂: 291.9547; found: 291.9545 (M).

### Example 849F 6-bromo-2,2-difluoro-2H,5H-indeno[5,6-d][1,3]dioxole

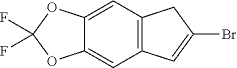 [SMILES: FC1(F)Oc2cc3c(cc2O1)CC(Br)=C3]

Using General procedure 7 and Example 849E as the appropriate indane, Example 849F was obtained. ¹H NMR (500 MHz, DMSO-d₆) δ ppm: 7.50 (m, 1H), 7.41 (s, 1H), 7.10 (m, 1H), 3.73 (m, 2H). HRMS calculated for C₁₀H₅O2BrF₂: 273.9441; found: 273.9436 (M).

### Example 849G 6″-bromo-2″,2″-difluoro-2″H-dispiro[[1,3]dioxolane-2,1′-cyclohexane-4′,5″-indeno[5,6-d][1,3]dioxole]

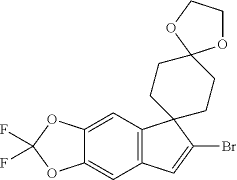 [SMILES: FC1(F)Oc2cc3c(cc2O1)C1(CCC2(CC1)OCCO2)C(Br)=C3]

Using General procedure 8a and Example 849F as the appropriate indene, Example 849G was obtained. ¹H NMR (500 MHz, DMSO-d₆) δ ppm: 7.77 (s, 1H), 7.42 (s, 1H), 7.03 (s, 1H), 3.95 (s, 4H), 2.09/1.21 (m+m, 4H), 2.09/1.86 (m+m, 4H). HRMS calculated for C₁₇H₁₅O₄BrF₂: 400.0122; found: 401.0193 (M+H).

### Example 849H 6′-bromo-2′,2′-difluoro-2′H-spiro[cyclohexane-1,5′-indeno[5,6-d][1,3]dioxol]-4-one

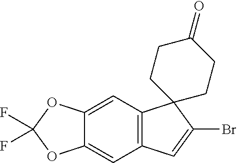 [SMILES: O=C1CCC2(CC1)C(Br)=Cc1cc3c(cc12)OC(F)(F)O3]

Using General procedure 9 and Example 849G as the appropriate ketal, Example 849H was obtained. ¹H NMR (500 MHz, DMSO-d₆) δ ppm: 8.13 (s, 1H), 7.46 (s, 1H), 7.09 (s, 1H), 2.91/2.49 (m+m, 4H), 2.19/1.61 (m+m, 4H). HRMS calculated for C₁₅H₁₁O₃BrF₂: 355.9860; found: 355.9845 (M).

### Example 849I 6″-bromo-2″,2″-difluoro-2″H-dispiro[imidazolidine-4,1′-cyclohexane-4′,5″-indeno[5,6-d][1,3]dioxole]-2,5-dione

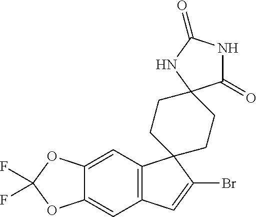 [SMILES: O=C1NC(=O)C2(CCC3(CC2)C(Br)=Cc2cc4c(cc23)OC(F)(F)O4)N1]

Using General procedure 14 and Example 849H as the appropriate ketone, Example 8491 was obtained as a mixture of diastereoisomers. ¹H NMR (500 MHz, DMSO-d₆) δ ppm: 10.83/10.67 (s, 1H), 8.96/8.46 (s, 1H), 7.98/7.56 (s, 1H), 7.47/7.40 (s, 1H), 7.06/7.05 (s, 1H), 2.48-1.13 (m, 8H). HRMS calculated for C₁₇H₁₃N₂O₄BrF₂: 426.0027; found: 426.0020 and 426.0032 (M).

### Example 849J 4-amino-6′-bromo-2′,2′-difluoro-2′H-spiro[cyclohexane-1,5′-indeno[5,6-d][1,3]dioxole]-4-carboxylic acid

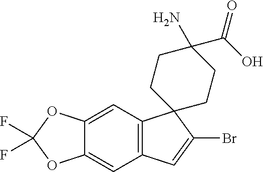 [SMILES: NC1(C(=O)O)CCC2(CC1)C(Br)=Cc1cc3c(cc12)OC(F)(F)O3]

Using General procedure 15 and Example 8491 as the appropriate hydantoin, Example 849J was obtained as a mixture of diastereoisomers. ¹H NMR (500 MHz, DMSO-d₆) δ ppm: 8.14/8.11 (s/s, 1H), 7.83 (br s, 2H), 7.44/7.40 (s/s, 1H), 7.03/7.00 (s/s, 1H), 2.58/2.45 (td/t, 2H), 2.24/1.83 (d/d, 2H), 2.06/1.92 (td/td, 2H), 1.12/1.09 (d/d, 2H).

### Example 849K 6′-bromo-4-(3-chloroanilino)-2′,2′-difluoro-2′H-spiro[cyclohexane-1,5′-indeno[5,6-d][1,3]dioxole]-4-carboxylic acid

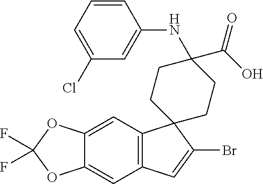 [SMILES: O=C(O)C1(Nc2cccc(Cl)c2)CCC2(CC1)C(Br)=Cc1cc3c(cc12)OC(F)(F)O3]

Using General procedure 16 and Example 849J as the appropriate amino acid and 1-chloro-3-iodo-benzene as the appropriate iodobenzene, Example 849K was obtained as a mixture of diastereoisomers. ¹H NMR (500 MHz, DMSO-d₆) δ ppm: 12.80 (br s, 1H), 7.76/7.67 (s/s, 1H), 7.45/7.42 (s/s, 1H), 7.13-7.06 (m, 1H), 7.03/7.02 (s/s, 1H), 6.71/6.64 (t/t, 1H), 6.63-6.54 (m, 2H), 2.41-2.00 (m, 6H), 1.00 (d, 2H).

### Example 849L methyl (1s,4s)-6′-bromo-4-(3-chloroanilino)-2′,2′-difluoro-2′H-spiro[cyclohexane-1,5′-indeno[5,6-d][1,3]dioxole]-4-carboxylate

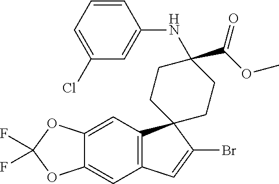 [SMILES: COC(=O)[C@]1(Nc2cccc(Cl)c2)CC[C@]2(CC1)C(Br)=Cc1cc3c(cc12)OC(F)(F)O3]

Using General procedure 17b and Example 849K as the appropriate amino acid, a mixture of diastereoisomers was obtained. The diastereoisomers were separated by flash chromatography using heptane and EtOAC as eluents. The diastereoisomer eluting later was collected as Example 849L. ¹H NMR (500 MHz, DMSO-d₆) δ ppm: 7.74 (s, 1H), 7.45 (s, 1H), 7.09 (t, 1H), 7.03 (s, 1H), 6.63 (t, 1H), 6.60 (dm, 1H), 6.50 (dm, 1H), 6.47 (s, 1H), 3.69 (s, 3H), 2.35/2.27 (td+d, 4H), 2.19/1.01 (td+d, 4H). HRMS calculated for C₂₃H₁₉NO₄F₂ClBr: 525.0154; found: 526.0222 (M+H).

### Example 849M methyl (1r,4R)-4-(3-chloroanilino)-2′,2′-difluoro-6′-{(2R)-3-[(4-methoxyphenyl)methoxy]-2-methylpropyl}-2′H-spiro[cyclohexane-1,5′-indeno[5,6-d][1,3]dioxole]-4-carboxylate

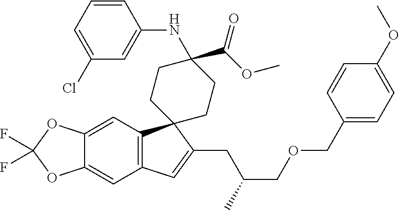 [SMILES: COc1ccc(COC[C@H](C)CC2=Cc3cc4c(cc3[C@]23CC[C@@](Nc2cccc(Cl)c2)(C(=O)OC)CC3)OC(F)(F)O4)cc1]

Using General procedure 27b and Example 849L as the appropriate 2-bromo-indene derivative and Preparation 3d as the appropriate Zn reagent, Example 849M was obtained. ¹H NMR (500 MHz, DMSO-d₆) δ ppm: 7.60 (s, 1H), 7.32 (s, 1H), 7.21 (m, 2H), 7.09 (t, 1H), 6.85 (m, 2H), 6.65 (t, 1H), 6.60 (dm, 1H), 6.49 (dm, 1H), 6.44 (m, 1H), 6.40 (s, 1H), 3.72 (s, 3H), 3.69 (s, 3H), 3.31/3.28 (m+m, 2H), 3.31/3.28 (d+d, 2H), 2.34/1.98 (dd+dd, 2H), 2.33/2.21 (m+m, 4H), 2.14 (m, 1H), 2.10/0.87 (m+m, 4H), 0.92 (d, 3H). HRMS calculated for C₃₅H₃₆NO₆F₂Cl: 639.2199; found: 640.2275 (M+H).

### Example 849N methyl (1r,4R)-4-(3-chloroanilino)-2′,2′-difluoro-6′-{(2R)-3-[(4-methoxyphenyl)methoxy]-2-methylpropyl}-6′,7′-dihydro-2′H-spiro[cyclohexane-1,5′-indeno[5,6-d][1,3]dioxole]-4-carboxylate

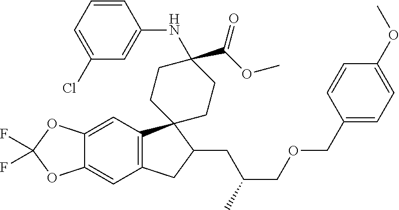

Using General procedure 19 and Example 849M as the appropriate indene, Example 849N was obtain as a mixture of diastereoisomers. ¹H NMR (500 MHz, DMSO-d₆) δ ppm: 7.33/7.31 (s, 1H), 7.23 (s, 1H), 7.21/7.18 (m, 2H), 7.06 (t, 1H), 6.86/6.79 (m, 2H), 6.62/6.60 (t, 1H), 6.57 (dm, 1H), 6.48/6.46 (dm, 1H), 6.31/6.29 (s, 1H), 4.42-4.30 (d+d, 2H), 3.72/3.68 (s, 3H), 3.65 (s, 3H), 3.36-3.09 (m, 2H), 2.95/2.50 (dd+dd, 2H), 2.50-0.94 (m, 11H), 2.15 (m, 1H), 0.92/0.87 (d, 3H). HRMS calculated for C₃₅H₃₈NO₆F₂Cl: 641.2356; found: 642.2423 and 642.2427 (M+H).

### Example 8490 methyl (1r,4S,6′S)-4-(3-chloroanilino)-2′,2′-difluoro-6′-[(2R)-3-hydroxy-2-methylpropyl]-6′,7′-dihydro-2′H-spiro[cyclohexane-1,5′-indeno[5,6-d][1,3]dioxole]-4-carboxylate

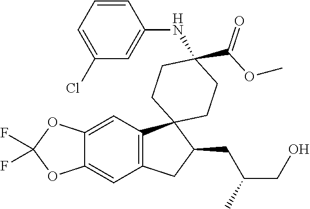 [SMILES: COC(=O)[C@]1(Nc2cccc(Cl)c2)CC[C@@]2(CC1)c1cc3c(cc1C[C@@H]2C[C@@H](C)CO)OC(F)(F)O3]

Using General procedure 28b and Example 849N as the appropriate PMB derivative, a mixture of diastereoisomers was obtained. The diastereoisomers were separated via chiral chromatography. Column: AD, 50×500 mm, 20 μm, Eluents: EtOH/heptane. The diastereoisomer eluting later was collected as Example 8490. ¹H NMR (500 MHz, DMSO-d₆) δ ppm: 7.33 (s, 1H), 7.25 (s, 1H), 7.06 (t, 1H), 6.59 (t, 1H), 6.57 (dd, 1H), 6.46 (dd, 1H), 6.30 (s, 1H), 4.45 (t, 1H), 3.65 (s, 3H), 3.23/3.18 (dd+dd, 2H), 2.95/2.51 (dd+dd, 2H), 2.47-1.36 (m, 8H), 2.17 (m, 1H), 1.54 (m, 1H), 1.30/1.04 (t+t, 2H), 0.85 (d, 3H). HRMS calculated for C₂₇H₃₀NO₅F₂Cl: 521.1781; found: 522.1857 (M+H).

### Example 849 (1r,4S,6′S)-4-(3-chloroanilino)-2′,2′-difluoro-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

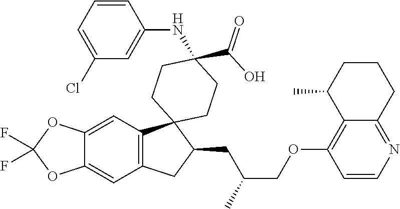 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OC(F)(F)O3]

Using General procedure 32 and Example 8490 as the appropriate indane and Preparation 2a1 as the appropriate alcohol, Example 849 was obtained. ¹H NMR (500 MHz, DMSO-d₆) δ ppm: 12.68 (br s, 1H), 8.14 (d, 1H), 7.28 (s, 1H), 7.25 (s, 1H), 7.04 (t, 1H), 6.76 (d, 1H), 6.62 (t, 1H), 6.54 (dm, 2H), 6.27 (br s, 1H), 3.86 (d, 2H), 3.02/2.54 (dd+dd, 2H), 3.01 (m, 1H), 2.75/2.64 (m+m, 2H), 2.47-1.37 (m, 12H), 2.24 (m, 1H), 1.98 (m, 1H), 1.45/1.30 (m+m, 2H), 1.04 (d, 3H), 0.99 (d, 3H). HRMS calculated for C₃₆H₃₉N₂O₅F₂Cl: 652.2515; found: 653.2582 (M+H).

### Example 850

### Example 850A (2E)-3-(2,3-dihydro-1-benzofuran-5-yl)prop-2-enoic acid

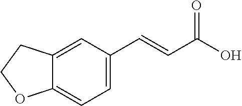 [SMILES: O=C(O)/C=C/c1ccc2c(c1)CCO2]

Pyrrolidine (1.41 ml, 0.85 g/cm³, 16.9 mmol, 0.1 eq) was added to a suspension of 2,3-dihydro-1-benzofuran-5-carbaldehyde (25 g, 169 mmol, 1 eq) and malonic acid (26.3 g, 254 mmol, 1.5 eq) in pyridine (75 ml). The mixture was heated at 100° C. for 8 h, cooled to rt and poured onto ice/water (500 mL). The suspension was acidified to pH 1 by the careful addition of 6 M aq. HCl solution, and this suspension was stirred for 30 min. The solids were removed by filtration, washed with water (300 mL) then heptane (2×150 mL) and finally dried in vacuo to afford Example 850A as a yellow solid (29.2 g, 154 mmol, 91%). ¹H NMR (400 MHz, DMSO-d₆) δ 12.20 (s, 1H), 7.61 (d, J=1.8 Hz, 1H), 7.53 (d, J=15.9 Hz, 1H), 7.43 (dd, J=8.4, 1.9 Hz, 1H), 6.80 (d, J=8.3 Hz, 1H), 6.34 (d, J=15.9 Hz, 1H), 4.59 (t, J=8.7 Hz, 2H), 3.20 (t, J=8.7 Hz, 2H).

### Example 850B 3-(2,3-dihydro-1-benzofuran-5-yl)propanoic acid

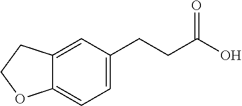 [SMILES: O=C(O)CCc1ccc2c(c1)CCO2]

To a suspension of Example 850A (3.00 g, 15.8 mmol, 1 eq) in EtOH (20 mL) was added 10% Pd/C (100 mg). The flask was evacuated and backfilled with N₂ (×3), then evacuated and filled with H₂. The reaction mixture was stirred at rt overnight. The reaction mixture was filtered, washed with EtOH, and the filtrate was concentrated under reduced pressure to afford Example 850B as a pale brown solid (3.00 g, 15.6 mmol, 99%). ¹H NMR (400 MHz, DMSO-d₆) δ ppm: 12.10 (s, 1H), 7.10-7.06 (m, 1H), 6.95-6.89 (m, 1H), 6.65 (d, J=8.1 Hz, 1H), 4.48 (t, J=8.7 Hz, 2H), 3.16-3.09 (m, 2H), 2.77-2.70 (m, 2H), 2.50-2.44 (m, 2H).

### Example 850C 2,3,5,6-tetrahydro-7H-indeno[5,6-b]furan-7-one

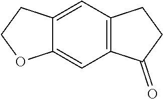 [SMILES: O=C1CCc2cc3c(cc21)OCC3]

Example 850B (87 g, 453 mmol, 1 eq) was added in portions to SOCl₂ (82.52 mL, 1.63 g/mL, 1131.31 mmol, 2.5 eq) at 0° C. After addition, the reaction mixture was slowly heated to 75° C. and continued heating until gas evolution ceased (1H). The reaction was cooled to rt and concentrated in vacuo and azeotroped with toluene (×3) to remove last traces of excess SOCl₂. The crude acid chloride obtained was dissolved in DCM (250 mL) and added dropwise to a suspension of AlCl₃ (66.38 g, 497.79 mmol, 1.1 eq) in DCM (750 mL) at 0° C. After addition stirring was continued at 0° C. and then at rt for 18 h. The reaction was poured onto ice/water (2 L) with stirring. This was further diluted with DCM (1 L) and then 2 M aq. HCl solution (1 L) was added to the mixture with rapid stirring. After stirring for a further 30 min the mixture was filtered through celite and the organics were separated. The aq. phase was extracted with further portions of DCM and the combined organic layers were washed with water, sat. aq. NH₄Cl solution, brine, dried (MgSO₄), filtered and concentrated in vacuo. Purification by automated flash chromatography (CombiFlash Rf, material split across 2×330 g RediSep™ silica cartridges) eluting with a gradient of 0-30% EtOAc in heptane afforded Example 850C as a white solid (48.86 g, 280.49 mmol, 62%). LRMS calculated for C_{II}H₁₀O₂: 174; found 175 (M+H). ¹H NMR (400 MHz, DMSO-d₆) δ ppm: 7.44-7.40 (m, 1H), 6.85 (s, 1H), 4.59 (t, J=8.6 Hz, 2H), 3.30-3.23 (m, 2H), 3.02-2.96 (m, 2H), 2.66-2.59 (m, 2H).

### Example 850D 6-bromo-2,3,5,6-tetrahydro-7H-indeno[5,6-b]furan-7-one

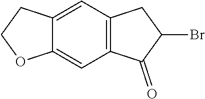 [SMILES: O=C1c2cc3c(cc2CC1Br)CCO3]

Using General procedure 5 and Example 850C as the appropriate indan-1-one, Example 850D was obtained. ¹H NMR (400 MHz, DMSO-d₆) δ ppm: 7.44-7.40 (m, 1H), 6.96 (s, 1H), 5.01 (dd, J=7.2, 2.8 Hz, 1H), 4.62 (t, J=8.7 Hz, 2H), 3.77 (dd, J=17.9, 7.2 Hz, 1H), 3.33-3.25 (m, 2H), 3.21 (dd, J=17.9, 2.8 Hz, 1H).

### Example 850E 6-bromo-3,5,6,7-tetrahydro-2H-indeno[5,6-b]furan-7-ol

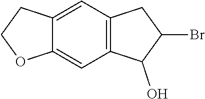 [SMILES: OC1c2cc3c(cc2CC1Br)CCO3]

Using General procedure 6 and Example 850D as the appropriate bromo-indan-1-one, Example 850E was obtained. ¹H NMR (400 MHz, DMSO-d₆) δ ppm: 7.10-7.05 (m, 1H), 6.67 (s, 1H), 5.69 (d, J=5.9 Hz, 1H), 4.87-4.79 (m, 2H), 4.56-4.45 (m, 2H), 3.33-3.26 (m, 1H), 3.17-3.04 (m, 3H).

### Example 850F 6-bromo-3,5-dihydro-2H-indeno[5,6-b]furan

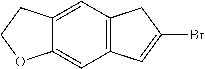 [SMILES: BrC1=Cc2cc3c(cc2C1)CCO3]

Using General procedure 7 and Example 850E as the appropriate indane and CHCl₃ instead of toluene, Example 850F was obtained. ¹H NMR (400 MHz, DMSO-d₆) δ ppm: 7.26-7.23 (m, 1H), 7.03-6.99 (m, 1H), 6.78-6.75 (m, 1H), 4.52 (t, J=8.7 Hz, 2H), 3.60-3.56 (m, 2H), 3.18-3.11 (m, 2H).

### Example 850G 6″-bromo-2″,3″-dihydrodispiro[[1,3]dioxolane-2,1′-cyclohexane-4′,5″-indeno[5,6-b]furan]

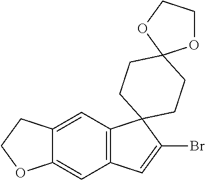 [SMILES: BrC1=Cc2cc3c(cc2C12CCC1(CC2)OCCO1)CCO3]

### Example 850H 6″-bromo-2″,3″-dihydrodispiro[[1,3]dioxolane-2,1′-cyclohexane-4′,7″-indeno[5,6-b]furan]

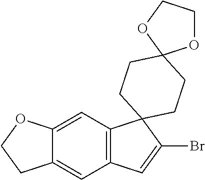 [SMILES: BrC1=Cc2cc3c(cc2C12CCC1(CC2)OCCO1)OCC3]

Using General procedure 8a and Example 850F as the appropriate indene, a mixture of regioisomers was obtained. The regioisomers were separated by automated flash chromatography (CombiFlash Rf, 330 g RediSep™ silica cartridge) eluting with a gradient of 0-80% DCM in heptane. The regioisomer eluting first was collected as Example 850G. ¹H NMR (400 MHz, DMSO-d₆) δ ppm: 7.56-7.51 (m, 1H), 6.93 (s, 1H), 6.76 (s, 1H), 4.52 (t, J=8.7 Hz, 2H), 4.00-3.91 (m, 4H), 3.23-3.15 (m, 2H), 2.16-2.02 (m, 4H), 1.93-1.79 (m, 2H), 1.22-1.07 (m, 2H).

The regioisomer eluting last was collected as Example 850H. ¹H NMR (400 MHz, DMSO-d₆) δ ppm: 7.22-7.19 (m, 1H), 7.03 (s, 1H), 6.92 (s, 1H), 4.54 (t, J=8.7 Hz, 2H), 3.99-3.91 (m, 4H), 3.21-3.13 (m, 2H), 2.13-1.97 (m, 4H), 1.93-1.81 (m, 2H), 1.26-1.12 (m, 2H).

### Example 850I 6′-bromo-2′,3′-dihydrospiro[cyclohexane-1,7′-indeno[5,6-b]furan]-4-one

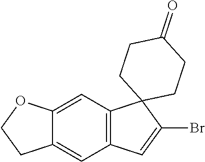 [SMILES: O=C1CCC2(CC1)C(Br)=Cc1cc3c(cc12)OCC3]

Using General procedure 9 and Example 850H as the appropriate ketal, Example 8501 was obtained. ¹H NMR (400 MHz, CDCl₃-d) δ ppm: 7.16-7.12 (m, 1H), 7.07 (s, 1H), 6.80-6.77 (m, 1H), 4.61 (t, J=8.7 Hz, 2H), 3.27-3.19 (m, 2H), 2.92-2.81 (m, 2H), 2.75-2.65 (m, 2H), 2.40-2.29 (m, 2H), 1.76-1.67 (m, 2H).

### Example 850J 6″-bromo-2″,3″-dihydrodispiro[imidazolidine-4,1′-cyclohexane-4′,7″-indeno[5,6-b]furan]-2,5-dione

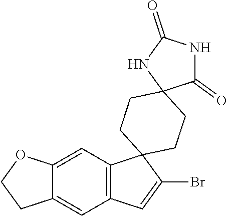 [SMILES: O=C1NC(=O)C2(CCC3(CC2)C(Br)=Cc2cc4c(cc23)OCC4)N1]

Using General procedure 14 and Example 8501 as the appropriate ketone, Example 850J was obtained as a mixture of diastereoisomers. ¹H NMR (400 MHz, DMSO-d₆) δ ppm: 8.55/8.42 (br s, 1H), 7.43-7.39/7.23-7.18 (m, 1H), 7.18-7.13 (m, 1H), 6.94-6.90 (m, 1H), 4.59-4.49 (m, 2H), 3.22-3.11 (m, 2H), 2.41-2.24 (m, 2H), 2.14-1.94 (m, 2H), 1.93-1.08 (m, 4H).

### Example 850K 4-amino-6′-bromo-2′,3′-dihydrospiro[cyclohexane-1,7′-indeno[5,6-b]furan]-4-carboxylic acid

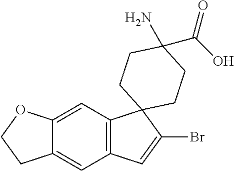 [SMILES: NC1(C(=O)O)CCC2(CC1)C(Br)=Cc1cc3c(cc12)OCC3]

Using General procedure 15 and Example 850J as the appropriate hydantoin, Example 850K was obtained as a mixture of diastereoisomers. LRMS calculated for C₁₇H₁₈BrNO₃: 363; found 364 (M+H). ¹H NMR (400 MHz, DMSO-d₆) δ ppm: 7.55-7.48 (m, 1H), 7.21-7.14 (m, 1H), 6.90/6.87 (s, 1H), 4.54 (t, J=8.7 Hz, 2H), 3.21-3.12 (m, 2H), 2.66-2.53/2.44-2.33 (m, 2H), 2.27-2.17/1.81-1.70 (m, 2H), 2.08-1.86 (m, 2H), 1.14-1.00 (m, 2H).

### Example 850L (1s,4s)-6′-bromo-4-(3-chloroanilino)-2′,3′-dihydrospiro[cyclohexane-1,7′-indeno[5,6-b]furan]-4-carboxylic acid

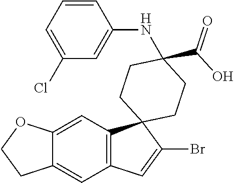 [SMILES: O=C(O)[C@]1(Nc2cccc(Cl)c2)CC[C@]2(CC1)C(Br)=Cc1cc3c(cc12)OCC3]

Using General procedure 16 and Example 850K as the appropriate amino acid and 1-chloro-3-iodo-benzene as the appropriate iodo-benzene and 2-(2-methylpropanyl)cyclohexan-1-one instead of ethyl-2-oxocyclohexanecarboxylate, a mixture of diastereoisomers was obtained. The diastereoisomers were separated by flash chromatography using heptane and EtOAc as eluents. The diastereoisomer eluting later was collected as Example 850L. ¹H NMR (400 MHz, DMSO-d₆) δ ppm: 12.82 (br s, 1H), 7.23-7.20 (m, 1H), 7.13-7.06 (m, 2H), 6.91 (s, 1H), 6.61 (t, J=2.1 Hz, 1H), 6.60-6.52 (m, 2H), 6.38 (br s, 1H), 4.55 (t, J=8.7 Hz, 2H), 3.23-3.13 (m, 2H), 2.42-2.28 (m, 2H), 2.28-2.10 (m, 4H), 1.03-0.92 (m, 2H).

### Example 850M methyl (1s,4s)-6′-bromo-4-(3-chloroanilino)-2′,3′-dihydrospiro[cyclohexane-1,7′-indeno[5,6-b]furan]-4-carboxylate

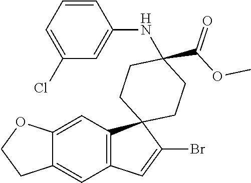 [SMILES: COC(=O)[C@]1(Nc2cccc(Cl)c2)CC[C@]2(CC1)C(Br)=Cc1cc3c(cc12)OCC3]

Using General procedure 17a and Example 850L as the appropriate amino acid, Example 850M was obtained. ¹H NMR (400 MHz, DMSO-d₆) δ ppm: 7.22-7.19 (m, 1H), 7.14-7.06 (m, 2H), 6.91 (s, 1H), 6.63-6.57 (m, 2H), 6.50-6.45 (m, 2H), 4.55 (t, J=8.7 Hz, 2H), 3.69 (s, 3H), 3.22-3.14 (m, 2H), 2.42-2.30 (m, 2H), 2.30-2.11 (m, 4H), 1.03-0.92 (m, 2H).

### Example 850N methyl (1r,4R)-4-(3-chloroanilino)-6′-{(2R)-3-[(4-methoxyphenyl)methoxy]-2-methylpropyl}-2′,3′-dihydrospiro[cyclohexane-1,7′-indeno[5,6-b]furan]-4-carboxylate

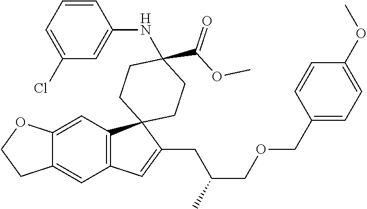 [SMILES: COc1ccc(COC[C@H](C)CC2=Cc3cc4c(cc3[C@]23CC[C@@](Nc2cccc(Cl)c2)(C(=O)OC)CC3)OCC4)cc1]

Using General procedure 27b and Example 850M as the appropriate 2-bromo-indene derivative and Preparation 3d as the appropriate Zn reagent, Example 850N was obtained. LRMS calculated for C₃₆H₄₀ClNO₅: 601; found 602 (M+H).

### Example 8500 methyl (1r,4R)-4-(3-chloroanilino)-6′-{(2R)-3-[(4-methoxyphenyl)methoxy]-2-methylpropyl}-2′,3′,5′,6′-tetrahydrospiro[cyclohexane-1,7′-indeno[5,6-b]furan]-4-carboxylate

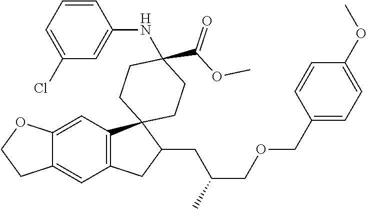

Using General procedure 19 and Example 850N as the appropriate indene and EtOH instead of EtOAc, Example 8500 was obtained as a mixture of diastereoisomers. LRMS calculated for C₃₆H₄₂ClNO₅: 603; found 604 (M+H).

### Example 850P methyl (1r,4R)-4-(3-chloroanilino)-6′-[(2R)-3-hydroxy-2-methylpropyl]-2′,3′,5′,6′-tetrahydrospiro[cyclohexane-1,7′-indeno[5,6-b]furan]-4-carboxylate

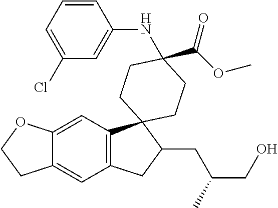

Using General procedure 28b and Example 8500 as the appropriate PMB derivative, Example 850P was obtained as a mixture of diastereoisomers. LRMS calculated for C₂₈H₃₄ClNO₄: 483; found 484 (M+H).

### Example 850Q methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-2′,3′,5′,6′-tetrahydrospiro[cyclohexane-1,7′-indeno[5,6-b]furan]-4-carboxylate

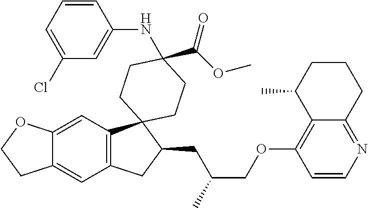 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)CCO3]

Using General procedure 30a and Example 850P as the appropriate indane and Preparation 2a1 as the appropriate alcohol, a mixture of diastereoisomers was obtained. The diastereoisomers were separated via chiral chromatography. Column: AD, 50×500 mm, 20 μm, Eluents: EtOH/heptane. The diastereoisomer eluting earlier was collected as Example 850Q. ¹H NMR (500 MHz, DMSO-d₆) δ ppm: 8.14 (d, 1H), 7.04 (t, 1H), 7.02 (s, 1H), 6.76 (d, 1H), 6.73 (s, 1H), 6.57 (t, 1H), 6.55 (dm, 1H), 6.43 (dm, 1H), 6.31 (s, 1H), 4.48 (t, 2H), 3.90/3.84 (dd+dd, 2H), 3.64 (s, 3H), 3.09 (t, 2H), 3.05 (m, 1H), 2.88/2.41 (dd+dd, 2H), 2.76/2.65 (dm+m, 2H), 2.45-1.17 (m, 14H), 2.11 (m, 1H), 1.97 (m, 1H), 1.05 (d, 3H), 1.03 (d, 3H). HRMS calculated for C₃₈H₄₅N₂O₄Cl: 628.3068; found: 629.3149 (M+H).

### Example 850 (1r,4S,6′S)-4-(3-chloroanilino)-6′-[(2R)-2-methyl-3-{[(5R)-5-methyl-5,6,7,8-tetrahydroquinolin-4-yl]oxy}propyl]-2′,3′,5′,6′-tetrahydrospiro[cyclohexane-1,7′-indeno[5,6-b]furan]-4-carboxylic acid

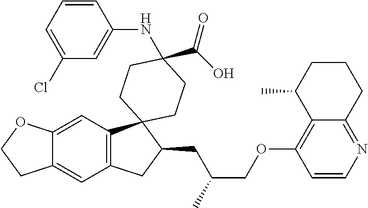 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC3]

Using General procedure 33a and Example 850Q as the appropriate ester, Example 850 was obtained. ¹H NMR (500 MHz, DMSO-d₆) δ ppm: 12.65 (br s, 3H), 8.14 (d, 1H), 7.03 (t, 1H), 7.02 (s, 1H), 6.77 (d, 1H), 6.71 (s, 1H), 6.59 (t, 1H), 6.53 (dm, 1H), 6.52 (dm, 1H), 6.22 (br s, 1H), 4.48 (t, 2H), 3.90/3.84 (dd+dd, 2H), 3.09 (t, 2H), 3.05 (m, 1H), 2.89/2.41 (dd+dd, 2H), 2.76/2.65 (dm+m, 2H), 2.43-1.26 (m, 14H), 2.12 (m, 1H), 1.97 (m, 1H), 1.05 (d, 3H), 1.03 (d, 3H). HRMS calculated for C₃₇H₄₃N₂O₄Cl: 614.2911; found: 615.2986 (M+H).

### Example 851

### Example 851A 6′-bromo-2′,3′-dihydrospiro[cyclohexane-1,5′-indeno[5,6-b]furan]-4-one

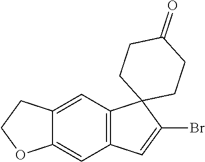 [SMILES: O=C1CCC2(CC1)C(Br)=Cc1cc3c(cc12)CCO3]

Using General procedure 9 and Example 850G as the appropriate ketal, Example 851A was obtained. LRMS calculated for C₁₆H₁₅BrO₂: 318; found 319 (M+H).

### Example 851B 6″-bromo-2″,3″-dihydrodispiro[imidazolidine-4,1′-cyclohexane-4′,5″-indeno[5,6-b]furan]-2,5-dione

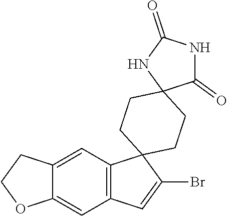 [SMILES: O=C1NC(=O)C2(CCC3(CC2)C(Br)=Cc2cc4c(cc23)CCO4)N1]

Using General procedure 14 and Example 851A as the appropriate ketone, Example 851B was obtained as a mixture of diastereoisomers. ¹H NMR (400 MHz, DMSO-d₆) δ ppm: 10.79/10.62 (br s, 1H), 8.91/8.44 (s, 1H), 7.76-7.73/7.56-7.52 (m, 1H), 6.96-6.94/6.94-6.93 (m, 1H), 6.80-6.78/6.74-6.73 (m, 1H), 4.58-4.49 (m, 2H), 3.26-3.18 (m, 2H), 2.41-1.11 (m, 8H).

### Example 851C 4-amino-6′-bromo-2′,3′-dihydrospiro[cyclohexane-1,5′-indeno[5,6-b]furan]-4-carboxylic acid

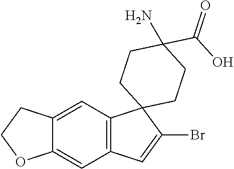 [SMILES: NC1(C(=O)O)CCC2(CC1)C(Br)=Cc1cc3c(cc12)CCO3]

Using General procedure 15 and Example 851B as the appropriate hydantoin, Example 851C was obtained as a mixture of diastereoisomers. LRMS calculated for C₁₇H₁₈BrlNO₃: 363; found 364 (M+H).

### Example 851D (1s,4s)-6′-bromo-4-(3-chloroanilino)-2′,3′-dihydrospiro[cyclohexane-1,5′-indeno[5,6-b]furan]-4-carboxylic acid

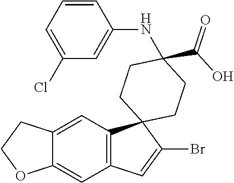 [SMILES: O=C(O)[C@]1(Nc2cccc(Cl)c2)CC[C@]2(CC1)C(Br)=Cc1cc3c(cc12)CCO3]

Using General procedure 16 and Example 851C as the appropriate amino acid and 1-chloro-3-iodo-benzene as the appropriate iodo-benzene and 2-(2-methylpropanyl)cyclohexan-1-one instead of ethyl-2-oxocyclohexanecarboxylate, a mixture of diastereoisomers was obtained. The diastereoisomers were separated by flash chromatography using heptane and EtOAC as eluents. The diastereoisomer eluting later was collected as Example 851D. ¹H NMR (400 MHz, DMSO-d₆) δ ppm: 12.78 (br s, 1H), 7.57-7.51 (m, 1H), 7.10 (t, J=8.1 Hz, 1H), 6.92 (s, 1H), 6.78 (s, 1H), 6.63 (t, J=2.1 Hz, 1H), 6.61-6.53 (m, 2H), 6.38 (br s, 1H), 4.53 (t, J=8.7 Hz, 2H), 3.26-3.18 (m, 2H), 2.43-2.30 (m, 2H), 2.30-2.11 (m, 4H), 1.02-0.91 (m, 2H).

### Example 851E methyl (1s,4s)-6′-bromo-4-(3-chloroanilino)-2′,3′-dihydrospiro[cyclohexane-1,5′-indeno[5,6-b]furan]-4-carboxylate

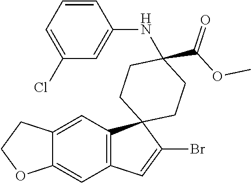 [SMILES: COC(=O)[C@]1(Nc2cccc(Cl)c2)CC[C@]2(CC1)C(Br)=Cc1cc3c(cc12)CCO3]

To a solution of Example 851D (4.34 g, 9.14 mmol, 1 eq) in DMF (35 mL) was added CsF (2.78 g, 18.28 mmol, 2 eq) and Mel (1.14 mL, 2.28 g/mL, 18.28 mmol, 2 eq). The reaction was heated at 40° C. for 3 h and after cooling to rt, partitioned between EtOAc and brine. The aq. phase was extracted with another portion of EtOAc and the combined organic layer was washed with water, brine, dried (MgSO₄), filtered and concentrated in vacuo. Purification by automated flash chromatography (CombiFlash Rf, 120 g RediSep™ silica cartridge) eluting with a gradient of 0-18% EtOAc in heptane afforded Example 851E as a yellow solid (4.11 g, 8.41 mmol, 92%). ¹H NMR (400 MHz, DMSO-d₆) δ ppm: 7.57-7.53 (m, 1H), 7.10 (t, J=8.0 Hz, 1H), 6.92 (s, 1H), 6.78 (s, 1H), 6.64-6.57 (m, 2H), 6.51-6.44 (m, 2H), 4.54 (t, J=8.7 Hz, 2H), 3.68 (s, 3H), 3.25-3.17 (m, 2H), 2.43-2.31 (m, 2H), 2.31-2.11 (m, 4H), 1.02-0.93 (m, 2H).

### Example 851F methyl (1r,4R)-4-(3-chloroanilino)-6′-{(2R)-3-[(4-methoxyphenyl)methoxy]-2-methylpropyl}-2′,3′-dihydrospiro[cyclohexane-1,5′-indeno[5,6-b]furan]-4-carboxylate

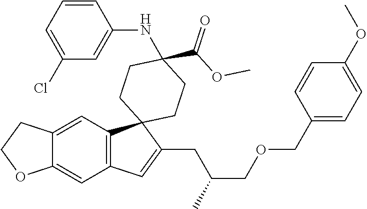 [SMILES: COc1ccc(COC[C@H](C)CC2=Cc3cc4c(cc3[C@]23CC[C@@](Nc2cccc(Cl)c2)(C(=O)OC)CC3)CCO4)cc1]

Using General procedure 27b and Example 851E as the appropriate 2-bromo-indene derivative and Preparation 3d as the appropriate Zn reagent, Example 851F was obtained. LRMS calculated for C₃₆H₄₀ClNO₅: 601; found 602 (M+H).

### Example 851G methyl (1r,4R)-4-(3-chloroanilino)-6′-{(2R)-3-[(4-methoxyphenyl)methoxy]-2-methylpropyl}-2′,3′,6′,7′-tetrahydrospiro[cyclohexane-1,5′-indeno[5,6-b]furan]-4-carboxylate

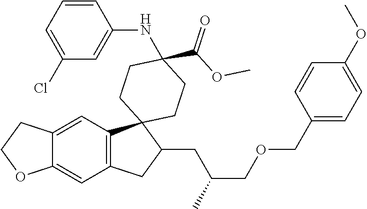

Using General procedure 19 and Example 851F as the appropriate indene, Example 851G was obtained as a mixture of diastereoisomers. LRMS calculated for C₃₈H₄0ClNO₅: 603; found 604 (M+H).

### Example 851H methyl (1r,4S,6′S)-4-(3-chloroanilino)-6′-[(2R)-3-hydroxy-2-methylpropyl]-2′,3′,5′,6-tetrahydrospiro[cyclohexane-1,7′-indeno[5,6-b]furan]-4-carboxylate

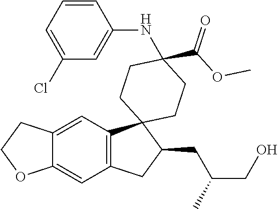 [SMILES: COC(=O)[C@]1(Nc2cccc(Cl)c2)CC[C@@]2(CC1)c1cc3c(cc1C[C@@H]2C[C@@H](C)CO)OCC3]

Using General procedure 28b and Example 851G as the appropriate PMB derivative, a mixture of diastereoisomers was obtained. The diastereoisomers were separated via chiral chromatography. Column: AD, 50×500 mm, 20 μm, Eluents: EtOH/heptane. The diastereoisomer eluting later was collected as Example 851H. ¹H NMR (500 MHz, DMSO-d₆) δ ppm: 7.16 (s, 1H), 7.06 (t, 1H), 6.59 (t, 1H), 6.58 (s, 1H), 6.56 (dm, 1H), 6.45 (dm, 1H), 6.29 (s, 1H), 4.47 (t, 2H), 4.44 (t, 1H), 3.64 (s, 3H), 3.20 (t, 2H), 3.12 (t, 2H), 2.83/2.40 (dd+dd, 2H), 2.47-1.26 (m, 8H), 2.06 (m, 1H), 1.54 (m, 1H), 1.31/1.03 (m+m, 2H), 0.84 (d, 3H). HRMS calculated for C₂₈H₃₄NO₄Cl: 483.2176; found: 484.2253 (M+H).

### Example 851 (1r,4S,6′S)-4-(3-chloroanilino)-6′-[(2R)-2-methyl-3-{[(5R)-5-methyl-5,6,7,8-tetrahydroquinolin-4-yl]oxy}propyl]-2′,3′,6′,7′-tetrahydrospiro[cyclohexane-1,5′-indeno[5,6-b]furan]-4-carboxylic acid

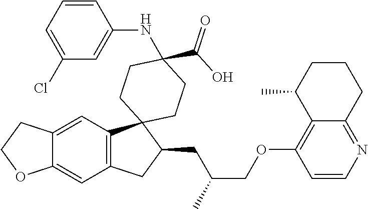 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)CCO3]

Using General procedure 32 and Example 851H as the appropriate indane and Preparation 2a1 as the appropriate alcohol, Example 851 was obtained. ¹H NMR (500 MHz, DMSO-d₆) δ ppm: 12.64 (br s, 1H), 8.14 (d, 1H), 7.17 (s, 1H), 7.04 (t, 1H), 6.76 (d, 1H), 6.61 (t, 1H), 6.58 (s, 1H), 6.53 (dm, 2H), 6.23 (br s, 1H), 4.47 (t, 2H), 3.90/3.84 (dd+dd, 2H), 3.12 (t, 2H), 3.05 (m, 1H), 2.90/2.43 (dd+dd, 2H), 2.76/2.65 (m+m, 2H), 2.48-1.28 (m, 12H), 2.11 (m, 1H), 1.97 (m, 1H), 1.46/1.32 (m+m, 2H), 1.05 (d, 3H), 1.03 (d, 3H). HRMS calculated for C₃₇H₄₃N₂O₄Cl: 614.2911; found: 615.2987 (M+H).

### Example 852

### Example 852A 2-bromo-3,5,6,7-tetrahydro-s-indacen-1 (2H)-one

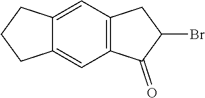 [SMILES: O=C1c2cc3c(cc2CC1Br)CCC3]

Using General procedure 5 and 3,5,6,7-tetrahydro-s-indacen-1 (2H)-one as the appropriate indan-1-one derivative, Example 852A was obtained. ¹H NMR (500 MHz, DMSO-d₆) δ ppm: 7.54 (s, 1H), 7.40 (s, 1H), 4.99 (dd, 1H), 3.81 (dd, 1H), 3.24 (dd, 1H), 2.99-2.85 (m, 4H), 2.11-2.01 (m, 2H).

### Example 852B 2-bromo-1,2,3,5,6,7-hexahydro-s-indacen-1-ol

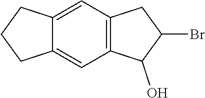 [SMILES: OC1c2cc3c(cc2CC1Br)CCC3]

Using General procedure 6 and Example 852A as the appropriate bromo-indan-1-one derivative, Example 852B was obtained. ¹H NMR (500 MHz, DMSO-d₆) δ ppm: 7.14 (s, 1H), 7.07 (s, 1H), 5.63 (br s, 1H), 4.88-4.80 (m, 2H), 3.33 (dd, 1H), 3.14-3.08 (m, 1H), 2.85-2.77 (m, 4H), 2.06-1.94 (m, 2H).

### Example 852C 6-bromo-1,2,3,5-tetrahydro-s-indacene

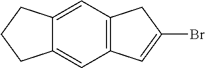 [SMILES: BrC1=Cc2cc3c(cc2C1)CCC3]

Using General procedure 7 and Example 852B as the appropriate indane derivative, Example 852C was obtained. ¹H NMR (500 MHz, DMSO-d₆) δ ppm: 7.25 (s, 1H), 7.19 (s, 1H), 7.03 (s, 1H), 3.61-3.57 (m, 2H), 2.87-2.79 (m, 4H), 2.05-1.97 (m, 2H).

### Example 852D 2″-bromo-6″,7″-dihydro-5″H-dispiro[[1,3]dioxolane-2,1′-cyclohexane-4′,1″-[s]indacene]

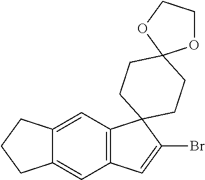 [SMILES: BrC1=Cc2cc3c(cc2C12CCC1(CC2)OCCO1)CCC3]

Using General procedure 8a and Example 852C as the appropriate indene derivative, Example 852D was obtained. ¹H NMR (500 MHz, DMSO-d₆) δ ppm: 7.52 (s, 1H), 7.19 (s, 1H), 6.96 (s, 1H), 3.96 (s, 4H), 2.88 (t, 2H), 2.84 (t, 2H), 2.14-2.05 (m, 4H), 2.05-1.97 (m, 2H), 1.89-1.81 (m, 2H), 1.19-1.11 (m, 2H).

### Example 852E 2′-bromo-6′,7′-dihydro-5′H-spiro[cyclohexane-1,1′-[s]indacen]-4-one

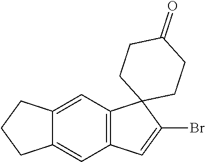 [SMILES: O=C1CCC2(CC1)C(Br)=Cc1cc3c(cc12)CCC3]

Using General procedure 9 and Example 852D as the appropriate ketal derivative, Example 852E was obtained. ¹H NMR (500 MHz, DMSO-d₆) δ ppm: 7.77 (s, 1H), 7.23 (s, 1H), 7.03 (s, 1H), 2.98-2.90 (m, 2H), 2.90-2.82 (m, 4H), 2.50-2.43 (m, 2H), 2.20 (td, 2H), 2.08-1.96 (m, 2H), 1.61-1.51 (m, 2H).

### Example 852F 2″-bromo-6″,7″-dihydro-5″H-dispiro[imidazolidine-4,1′-cyclohexane-4′,1″-[s]indacene]-2,5-dione

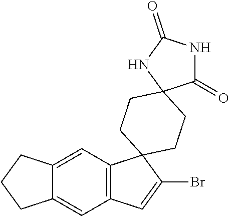 [SMILES: O=C1NC(=O)C2(CCC3(CC2)C(Br)=Cc2cc4c(cc23)CCC4)N1]

Using General procedure 14 and Example 852E as the appropriate ketone derivative, Example 852F was obtained as as a mixture of diastereoisomers. ¹H NMR (500 MHz, DMSO-d₆) δ ppm: 10.77/10.59 (s, 1H), 8.88/8.47 (s, 1H), 7.75/7.56 (s, 1H), 7.20/7.15 (s, 1H), 6.98/6.97 (s, 1H), 2.94-2.80 (m, 4H), 2.42-2.32 (m, 2H), 2.14-2.06 (m, 2H), 2.06-1.98 (m, 2H), 1.79-1.70 (m, 2H), 1.21-1.09 (m, 2H).

### Example 852G 4-amino-2′-bromo-6′,7′-dihydro-5′H-spiro[cyclohexane-1,1′-[s]indacene]-4-carboxylic acid

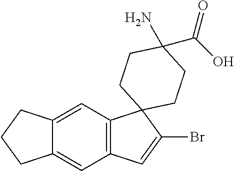 [SMILES: NC1(C(=O)O)CCC2(CC1)C(Br)=Cc1cc3c(cc12)CCC3]

Using General procedure 15 and Example 852F as the appropriate hydantoin derivative, Example 852G was obtained as as a mixture of diastereoisomers. ¹H NMR (500 MHz, DMSO-d₆) δ ppm: 7.86 (s, 1H), 7.18/7.15 (s, 1H), 6.95/6.91 (s, 1H), 2.92-2.77 (m, 4H), 2.68-2.57 (m, 2H), 2.10-1.95 (m, 4H), 1.80-1.68 (m, 2H), 1.13-1.00 (m, 2H).

### Example 852H methyl (1s,4s)-2′-bromo-4-(3-chloroanilino)-6′,7′-dihydro-5′H-spiro[cyclohexane-1,1′-[s]indacene]-4-carboxylate

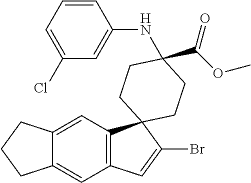 [SMILES: COC(=O)[C@]1(Nc2cccc(Cl)c2)CC[C@]2(CC1)C(Br)=Cc1cc3c(cc12)CCC3]

Using General procedure 16 and Example 852G as the appropriate amino acid derivative a mixture of diastereoisomers was obtained. It was treated as described in General procedure 17b. The formed diastereoisomers were separated via flash chromatography using heptane and EtOAc as eluents. The diastereoisomer eluting later was collected as Example 852H. ¹H NMR (500 MHz, DMSO-d₆) δ ppm: 7.55 (s, 1H), 7.20 (s, 1H), 7.09 (t, 1H), 6.96 (s, 1H), 6.61 (t, 1H), 6.59 (dd, 1H), 6.48 (s, 1H), 6.47 (dd, 1H), 3.68 (s, 3H), 2.89 (t, 2H), 2.84 (t, 2H), 2.38/2.25 (t+d, 4H), 2.18/0.97 (t+d, 4H), 2.03 (quint, 2H). HRMS calculated for C₂₅H₂₅BrClNO₂: 485.0757; found: 486.0829 (M+H).

### Example 8521 methyl (1r,4R)-4-(3-chloroanilino)-2′-{(2R)-3-[(4-methoxyphenyl)methoxy]-2-methylpropyl}-6′,7′-dihydro-5′H-spiro[cyclohexane-1,1′-[s]indacene]-4-carboxylate

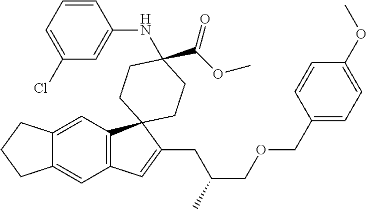 [SMILES: COc1ccc(COC[C@H](C)CC2=Cc3cc4c(cc3[C@]23CC[C@@](Nc2cccc(Cl)c2)(C(=O)OC)CC3)CCC4)cc1]

Using General procedure 27b and Example 852H as the appropriate 2-bromo-indene derivative and Preparation 3d as the appropriate Zn reagent, Example 8521 was obtained. ¹H NMR (500 MHz, DMSO-d₆) δ ppm: 7.48 (s, 1H), 7.21 (m, 2H), 7.09 (t, 1H), 7.09 (s, 1H), 6.85 (m, 2H), 6.64 (t, 1H), 6.59 (dm, 1H), 6.47 (dm, 1H), 6.40 (s, 1H), 6.36 (s, 1H), 4.39/4.35 (d+d, 2H), 3.72 (s, 3H), 3.68 (s, 3H), 3.33/3.27 (dd+dd, 2H), 2.87 (t, 2H), 2.81 (t, 2H), 2.37/2.18 (m+m, 4H), 2.31/1.95 (m+m, 2H), 2.13 (m, 1H), 2.09/0.84 (m+m, 4H), 2.02 (m, 2H), 0.93 (d, 3H). HRMS calculated for C₃₇H₄₂ClNO₄: 599.2802; found: 600.2853 (M+H).

### Example 852J methyl (1r,2′S,4S)-4-(3-chloroanilino)-2′-[(2R)-3-hydroxy-2-methylpropyl]-3′,5′,6′,7′-tetrahydro-2′H-spiro[cyclohexane-1,1′-[s]indacene]-4-carboxylate

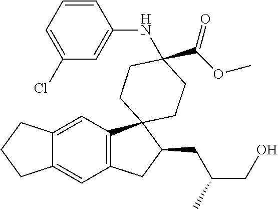 [SMILES: COC(=O)[C@]1(Nc2cccc(Cl)c2)CC[C@@]2(CC1)c1cc3c(cc1C[C@@H]2C[C@@H](C)CO)CCC3]

Using General procedure 19 and Example 8521 as the appropriate indene derivative, a mixture of diastereoisomers was obtained. It was treated as described in General procedure 28b. The obtained diastereoisomers were separated via chiral chromatography. Column: AS, 100×500 mm, 20 μm, Eluents: 15:85 nPrOH/heptane. The diastereoisomer eluting earlier was collected as Example 852J. ¹H NMR (500 MHz, DMSO-d₆) δ ppm: 7.16 (s, 1H), 7.06 (t, 1H), 7.02 (s, 1H), 6.59 (t, 1H), 6.56 (dm, 1H), 6.45 (dm, 1H), 6.29 (s, 1H), 4.43 (t, 1H), 3.64 (s, 3H), 3.20 (m, 2H), 2.86/2.42 (dd+dd, 2H), 2.81 (t, 2H), 2.77 (t, 2H), 2.47-0.98 (m, 10H), 2.08 (m, 1H), 1.99 (m, 2H), 1.55 (m, 1H), 0.84 (d, 3H). HRMS calculated for C₂₉H₃₆ClNO₃: 481.2384; found: 482.2467 (M+H).

### Example 852 (1r,2′S,4S)-4-(3-chloroanilino)-2′-[(2R)-2-methyl-3-{[(5R)-5-methyl-5,6,7,8-tetrahydroquinolin-4-yl]oxy}propyl]-3′,5′,6′,7′-tetrahydro-2′H-spiro[cyclohexane-1,1′-[s]indacene]-4-carboxylic acid

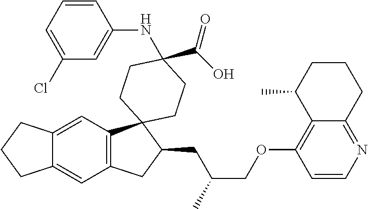 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)CCC3]

Using General procedure 32 and Example 852J as the appropriate indane and Preparation 2a1 as the appropriate alcohol, Example 852 was obtained. ¹H NMR (500 MHz, DMSO-d₆) δ ppm: 12.65 (br s, 1H), 8.14 (d, 1H), 7.17 (s, 1H), 7.04 (t, 1H), 7.02 (s, 1H), 6.76 (d, 1H), 6.62 (t, 1H), 6.53 (dm, 2H), 6.23 (br s, 1H), 3.89/3.84 (dd+dd, 2H), 3.04 (m, 1H), 2.92/2.45 (dd+dd, 2H), 2.81 (t, 2H), 2.77 (t, 2H), 2.76/2.65 (m+m, 2H), 2.49-1.33 (m, 12H), 2.13 (m, 1H), 1.99 (m, 2H), 1.98 (m, 1H), 1.46/1.31 (m+m, 2H), 1.04 (d, 3H), 1.04 (d, 3H). HRMS calculated for C₃₈H₄₅N₂O₃Cl: 612.3119; found: 613.3193 (M+H).

### Example 853

### Example 853A methyl (1r,4R)-4-(3-chloroanilino)-2′-{(2R)-3-[(4-methoxyphenyl)methoxy]-2-methylpropyl}-5′-methylspiro[cyclohexane-1,1′-indene]-4-carboxylate

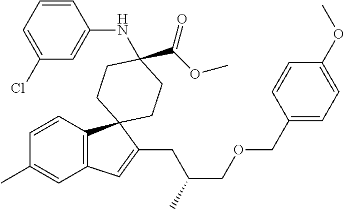 [SMILES: COc1ccc(COC[C@H](C)CC2=Cc3cc(C)ccc3[C@]23CC[C@@](Nc2cccc(Cl)c2)(C(=O)OC)CC3)cc1]

Using General procedure 27b and Example 824K as the appropriate 2-bromo-indene and Preparation 3d as the appropriate Zn reagent, Example 853A was obtained as a yellow gum. LRMS calculated for C₃₅H₄₀ClNO₄: 573; found: 574 (M+H).

### Example 853B methyl (1r,4R)-4-(3-chloroanilino)-2′-[(2R)-3-hydroxy-2-methylpropyl]-5′-methylspiro[cyclohexane-1,1′-indene]-4-carboxylate

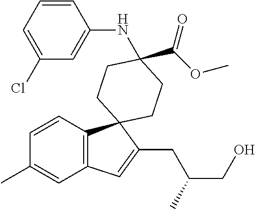 [SMILES: COC(=O)[C@]1(Nc2cccc(Cl)c2)CC[C@]2(CC1)C(C[C@@H](C)CO)=Cc1cc(C)ccc12]

Using General procedure 28a and Example 853A as the appropriate PMB derivative, Example 853B was obtained as a brown gum. LRMS calculated for C₂₇H₃₂ClNO₃: 453; found: 454 (M+H).

### Example 853C methyl (1r,2′S,4S)-4-(3-chloroanilino)-2′-[(2R)-3-hydroxy-2-methylpropyl]-5′-methyl-2′,3′-dihydrospiro[cyclohexane-1,1′-indene]-4-carboxylate

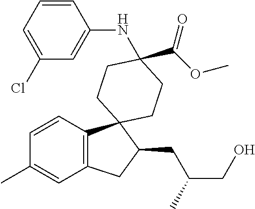 [SMILES: COC(=O)[C@]1(Nc2cccc(Cl)c2)CC[C@@]2(CC1)c1ccc(C)cc1C[C@@H]2C[C@@H](C)CO]

Using General procedure 19 and Example 853B as the appropriate indene, a mixture of diastereoisomers was obtained. They were purified and separated by preparative HPLC eluting with a gradient of 5-94% MeCN in water. The diastereoisomer eluting earlier was collected as Example 853C, isolated as a colourless gum. LRMS calculated for C₂₇H₃₄ClNO₃: 455; found: 456 (M+H).

### Example 853D methyl (1r,2′S,4S)-4-(3-chloroanilino)-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate

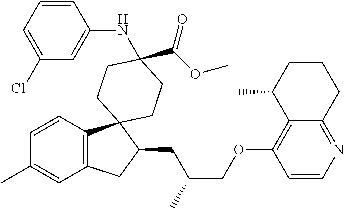 [SMILES: COC(=O)[C@]1(Nc2cccc(Cl)c2)CC[C@@]2(CC1)c1ccc(C)cc1C[C@@H]2C[C@@H](C)COc1ccnc2c1[C@H](C)CCC2]

Using General procedure 30a and Example 853C as the appropriate indane and Preparation 2a1 as the appropriate alcohol, Example 853D was obtained as a colourless glass. LRMS calculated for C₃₇H₄₅ClN₂O₃: 600; found: 601 (M+H).

### Example 853 (1r,2′S,4S)-4-(3-chloroanilino)-5′-methyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

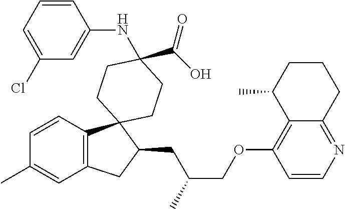 [SMILES: Cc1ccc2c(c1)C[C@H](C[C@@H](C)COc1ccnc3c1[C@H](C)CCC3)[C@]21CC[C@](Nc2cccc(Cl)c2)(C(=O)O)CC1]

Using General procedure 33a and Example 853D as the appropriate ester, Example 853 was obtained as a white solid. LRMS calculated for C₃₆H₄₃ClN₂O₃: 586; found: 587 (M+H). ¹H NMR (400 MHz, DMSO-d₆) δ ppm: 8.15 (d, J=5.6 Hz, 1H), 7.25 (d, J=7.8 Hz, 1H), 7.07-6.99 (m, 2H), 6.99-6.93 (m, 1H), 6.77 (d, J=5.6 Hz, 1H), 6.62 (t, J=2.1 Hz, 1H), 6.57-6.51 (m, 2H), 6.21 (br s, 1H), 3.95-3.81 (m, 2H), 3.10-3.01 (m, 1H), 2.95 (dd, J=15.6, 7.1 Hz, 1H), 2.82-2.72 (m, 1H), 2.66 (ddd, J=17.6, 11.0, 6.4 Hz, 1H), 2.51-2.36 (m, 2H), 2.26 (s, 3H), 2.17-2.05 (m, 2H), 2.05-1.93 (m, 2H), 1.93-1.56 (m, 7H), 1.54-1.40 (m, 2H), 1.40-1.28 (m, 2H), 1.08-1.00 (m, 6H).

### Example 854 (1r,2′S,4S)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

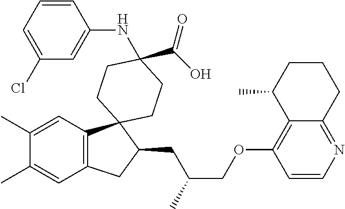 [SMILES: Cc1cc2c(cc1C)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Example 733D as the appropriate indane and Preparation 2a1 as the appropriate alcohol, Example 854 was obtained. ¹H NMR (500 MHz, DMSO-d₆) δ ppm: 12.63 (br s, 1H), 8.14 (d, 1H), 7.10 (s, 1H), 7.04 (t, 1H), 6.95 (s, 1H), 6.76 (d, 1H), 6.62 (t, 1H), 6.53 (dm, 2H), 6.22 (br s, 1H), 3.89/3.84 (dd+dd, 2H), 3.04 (m, 1H), 2.91/2.44 (dd+dd, 2H), 2.82-1.25 (m, 17H), 2.18 (s, 3H), 2.15 (s, 3H), 2.11 (m, 1H), 1.03 (d, 3H), 1.03 (d, 3H). HRMS calculated for C₃₇H₄₅N₂O₃Cl: 600.3119; found: 601.3203 (M+H).

### Example 855 (1r,2′R,4R)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

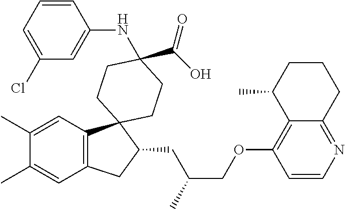 [SMILES: Cc1cc2c(cc1C)[C@]1(CC[C@@](Nc3cccc(Cl)c3)(C(=O)O)CC1)[C@H](C[C@@H](C)COc1ccnc3c1[C@H](C)CCC3)C2]

Using General procedure 32 and Example 733E as the appropriate indane and Preparation 2a1 as the appropriate alcohol, Example 855 was obtained. ¹H NMR (500 MHz, DMSO-d₆) δ ppm: 12.65 (br s, 1H), 8.13 (d, 1H), 7.12 (s, 1H), 7.03 (t, 1H), 6.95 (s, 1H), 6.77 (d, 1H), 6.60 (t, 1H), 6.53 (dm, 1H), 6.50 (dm, 1H), 6.10 (br s, 1H), 4.00/3.86 (dd+dd, 2H), 3.01 (m, 1H), 2.91/2.49 (dd+dd, 2H), 2.78-1.14 (m, 17H), 2.18 (s, 3H), 2.15 (s, 3H), 2.05 (m, 1H), 1.09 (d, 3H), 1.08 (d, 3H). HRMS calculated for C₃₇H₄₅N₂O₃Cl: 600.3119; found: 601.3181 (M+H).

### Example 856 (1r,4R)-6′-acetyl-4-(3-chloroanilino)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

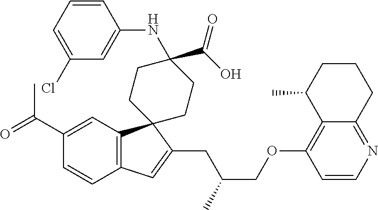 [SMILES: CC(=O)c1ccc2c(c1)[C@]1(CC[C@@](Nc3cccc(Cl)c3)(C(=O)O)CC1)C(C[C@@H](C)COc1ccnc3c1[C@H](C)CCC3)=C2]

Using General procedure 32 and Preparation 17aC as the appropriate indene and Preparation 2a1 as the appropriate alcohol, Example 856 was obtained. ¹H NMR (500 MHz, DMSO-d₆) δ ppm: 12.87 (br s, 1H), 8.26 (d, 1H), 8.14 (d, 1H), 7.92 (dd, 1H), 7.42 (d, 1H), 7.08 (t, 1H), 6.77 (d, 1H), 6.68 (s, 1H), 6.64 (t, 1H), 6.59 (dd, 1H), 6.55 (dd, 1H), 6.39 (br s, 1H), 4.03/3.95 (dd+dd, 2H), 3.07 (m, 1H), 2.76/2.65 (m+m, 2H), 2.57 (s, 3H), 2.57/2.56 (m+m, 2H), 2.47 (m, 1H), 2.43-0.9 (m, 8H), 1.80/1.70 (m+m, 2H), 1.61/1.55 (m+m, 2H), 1.13 (d, 3H), 1.13 (d, 3H). HRMS calculated for C₃₇H₄₁N₂O₄Cl: 612.2755; found: 613.2821 (M+H).

### Example 857 (1r,4R)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

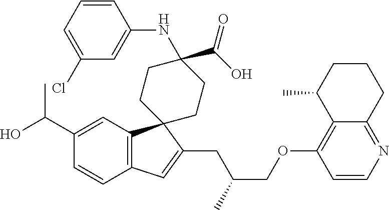 [SMILES: CC(O)c1ccc2c(c1)[C@]1(CC[C@@](Nc3cccc(Cl)c3)(C(=O)O)CC1)C(C[C@@H](C)COc1ccnc3c1[C@H](C)CCC3)=C2]

Using General procedure 30a and Preparation 17aC as the appropriate indene and Preparation 2a1 as the appropriate alcohol, an intermediate was obtained which was treated as described in General procedure 36 to obtain a mixture of diastereoisomers. It was further purified via prep RP-HPLC using 25 mM aq. NH₄HCO₃ solution and MeCN as eluents. Then it was hydrolyzed according to General procedure 33a to obtain Example 857 as a mixture of diastereoisomers. ¹H NMR (500 MHz, DMSO-d₆) δ ppm: 12.76 (br s, 1H), 8.13 (d, 1H), 7.69 (br s, 1H), 7.21 (d, 1H), 7.19 (dd, 1H), 7.08 (t, 1H), 6.77 (d, 1H), 6.64 (t, 1H), 6.57 (dm, 1H), 6.56 (dm, 1H), 6.51 (s, 1H), 6.35 (br s, 1H), 5.10 (d, 1H), 4.71 (m, 1H), 4.02/3.93 (dd+dd, 2H), 3.07 (m, 1H), 2.75/2.64 (m+m, 2H), 2.52-0.77 (m, 15H), 1.34 (d, 3H), 1.12 (d, 3H), 1.10 (d, 3H). HRMS calculated for C₃₇H₄₃N₂O₄Cl: 614.2911; found: 615.2978 (M+H) and 615.2986 (M+H).

### Example 858 (1r,2′S,4S)-6′-acetyl-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

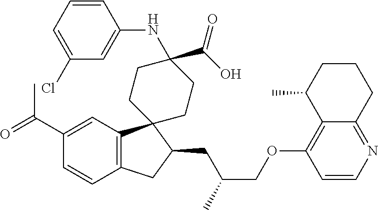 [SMILES: CC(=O)c1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Preparation 17a as the appropriate indane and Preparation 2a1 as the appropriate alcohol, Example 858 was obtained. ¹H NMR (500 MHz, DMSO-d₆) δ ppm: 12.74 (br s, 1H), 8.14 (d, 1H), 7.90 (d, 1H), 7.82 (dd, 1H), 7.35 (d, 1H), 7.05 (t, 1H), 6.86 (d, 1H), 6.62 (t, 1H), 6.54 (dm, 2H), 6.27 (br s, 1H), 3.90/3.85 (dd+dd, 2H), 3.09/2.61 (dd+dd, 2H), 3.03 (m, 1H), 2.76/2.64 (m+m, 2H), 2.56 (s, 3H), 2.50-1.38 (m, 12H), 2.22 (m, 1H), 2.00 (m, 1H), 1.45/1.35 (m+m, 2H), 1.05 (d, 3H), 1.03 (d, 3H). HRMS calculated for C₃₇H₄₃N₂O₄Cl: 614.2911; found: 615.2977 (M+H).

### Example 859 and Example 860

### Example 859A methyl (1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate, diastereoisomer 1

and

### Example 860A methyl (1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylate, diastereoisomer 2

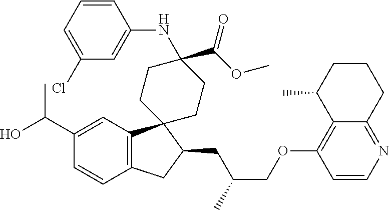 [SMILES: COC(=O)[C@]1(Nc2cccc(Cl)c2)CC[C@@]2(CC1)c1cc(C(C)O)ccc1C[C@@H]2C[C@@H](C)COc1ccnc2c1[C@H](C)CCC2]

Using General procedure 30a and Preparation 17a as the appropriate indane derivative and Preparation 2a1 as the appropriate alcohol, an intermediate was obtained which was treated as described in General procedure 36 to obtain a mixture of diastereoisomers. They were separated by chiral chromatography. Column: AD, 50×500 mm, 20 μm, Eluents: 10:90 MeCN/EtOH+0.05% DEA. The diastereoisomer eluting earlier was collected as Example 859A. ¹H NMR (500 MHz, DMSO-d₆) δ ppm: 8.14 (d, 1H), 7.34 (br s, 1H), 7.11 (m, 2H), 7.05 (t, 1H), 6.76 (d, 1H), 6.59 (t, 1H), 6.56 (dm, 1H), 6.45 (dm, 1H), 6.33 (s, 1H), 4.69 (q, 1H), 3.90/3.84 (dd+dd, 2H), 3.65 (s, 3H), 3.04 (m, 1H), 2.96/2.51 (dd+dd, 2H), 2.76/2.66 (dm+m, 2H), 2.52-1.33 (m, 8H), 2.15 (m, 1H), 2.00 (m, 1H), 1.79/1.73 (m+m, 2H), 1.66/1.60 (m+m, 2H), 1.46/1.32 (m+m, 2H), 1.31 (d, 3H), 1.04 (d, 3H), 1.04 (d, 3H). HRMS calculated for C₃₈H₄₇ClN₂O₄: 630.3224; found: 631.3277 (M+H).

The diastereoisomer eluting later was collected as Example 860A. ¹H NMR (500 MHz, DMSO-d₆) δ ppm: 8.14 (d, 1H), 7.37 (br s, 1H), 7.12 (d, 1H), 7.09 (dd, 1H), 7.04 (t, 1H), 6.76 (d, 1H), 6.59 (t, 1H), 6.56 (dm, 1H), 6.45 (dm, 1H), 6.34 (s, 1H), 4.68 (q, 1H), 3.90/3.85 (dd+dd, 2H), 3.65 (s, 3H), 3.04 (m, 1H), 2.96/2.50 (dd+dd, 2H), 2.76/2.66 (dm+m, 2H), 2.53-1.33 (m, 8H), 2.15 (m, 1H), 2.00 (m, 1H), 1.79/1.73 (m+m, 2H), 1.67/1.59 (m+m, 2H), 1.47/1.34 (m+m, 2H), 1.30 (d, 3H), 1.04 (d, 3H), 1.04 (d, 3H). HRMS calculated for C₃₈H₄₇ClN₂O₄: 630.3224; found: 631.3316 (M+H).

### Example 859 (1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 1

and

### Example 860 (1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid, diastereoisomer 2

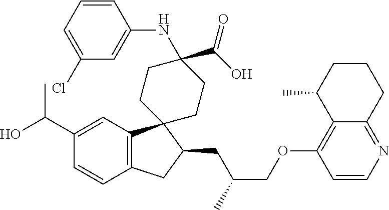 [SMILES: CC(O)c1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 33a and Example 859A as the appropriate ester derivative, Example 859 was obtained. ¹H NMR (400 MHz, DMSO-d₆) δ ppm: 12.66 (br s, 1H), 8.14 (d, 1H), 7.32 (m, 1H), 7.12 (d, 1H), 7.10 (dd, 1H), 7.04 (t, 1H), 6.76 (d, 1H), 6.63 (t, 1H), 6.55 (dd, 1H), 6.53 (dd, 1H), 6.23 (br s, 1H), 5.08 (d, 1H), 4.68 (dq, 1H), 3.90/3.84 (dd+dd, 2H), 3.04 (m, 1H), 2.97/2.49 (dd+dd, 2H), 2.76/2.66 (m+m, 2H), 2.46-1.45 (m, 8H), 2.17 (m, 1H), 2.00 (m, 1H), 1.79/1.73 (m+m, 2H), 1.67/1.60 (m+m, 2H), 1.45/1.33 (m+m, 2H), 1.30 (d, 3H), 1.05 (d, 3H), 1.03 (d, 3H). HRMS calculated for C₃₇H₄₅N₂O₄Cl: 616.3068; found: 617.3150 (M+H).

Using General procedure 33a and Example 860A as the appropriate ester derivative, Example 860 was obtained. ¹H NMR (400 MHz, DMSO-d₆) δ ppm: 12.67 (br s, 1H), 8.14 (d, 1H), 7.32 (m, 1H), 7.11 (d, 1H), 7.08 (dd, 1H), 7.04 (t, 1H), 6.76 (d, 1H), 6.63 (t, 1H), 6.55 (dd, 1H), 6.53 (dd, 1H), 6.21 (br s, 1H), 5.08 (d, 1H), 4.67 (dq, 1H), 3.90/3.84 (dd+dd, 2H), 3.04 (m, 1H), 2.97/2.50 (dd+dd, 2H), 2.76/2.65 (m+m, 2H), 2.48-1.42 (m, 8H), 2.16 (m, 1H), 1.98 (m, 1H), 1.80/1.73 (m+m, 2H), 1.66/1.60 (m+m, 2H), 1.46/1.33 (m+m, 2H), 1.29 (d, 3H), 1.05 (d, 3H), 1.04 (d, 3H). HRMS calculated for C₃₇H₄₅N₂O₄Cl: 616.3068; found: 617.3124 (M+H).

### Example 861 and Example 862

### Example 861A methyl (1r,2′S,4S)-6′-acetyl-2′-[(2R)-3-{[(5R,8R)-8-{[tert-butyl(dimethyl)silyl]oxy}-5-methyl-5,6,7,8-tetrahydroquinolin-4-yl]oxy}-2-methylpropyl]-4-(3-chloroanilino)-2′,3′-dihydrospiro[cyclohexane-1,1′-indene]-4-carboxylate

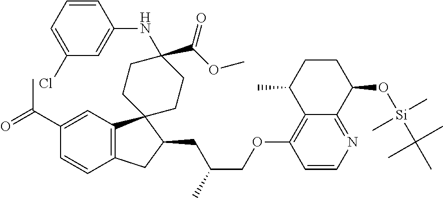 [SMILES: COC(=O)[C@]1(Nc2cccc(Cl)c2)CC[C@@]2(CC1)c1cc(C(C)=O)ccc1C[C@@H]2C[C@@H](C)COc1ccnc2c1[C@H](C)CC[C@H]2O[Si](C)(C)C(C)(C)C]

Using General procedure 30a and Preparation 17a as the appropriate indane derivative and Example 770A as the appropriate alcohol, Example 861A was obtained. ¹H NMR (500 MHz, DMSO-d₆) δ ppm: 8.25 (d, 1H), 7.92 (br s, 1H), 7.82 (dd, 1H), 7.36 (d, 1H), 7.05 (t, 1H), 6.85 (d, 1H), 6.59 (t, 1H), 6.56 (dm, 1H), 6.45 (dm, 1H), 6.36 (s, 1H), 4.63 (m, 1H), 3.92/3.89 (dd+dd, 2H), 3.66 (s, 3H), 3.08/2.59 (dd+dd, 2H), 3.03 (m, 1H), 2.56 (s, 3H), 2.53-1.31 (m, 8H), 2.21 (m, 1H), 2.11/1.41 (m+m, 2H), 2.01 (m, 1H), 1.98/1.75 (m+m, 2H), 1.47/1.38 (m+m, 2H), 1.06 (d, 3H), 1.00 (d, 3H), 0.82 (s, 9H), 0.17/−0.05 (s+s, 6H). HRMS calculated for C₄₄H₅₉ClN₂O₅Si: 758.3882; found: 759.3967 (M+H).

### Example 861B methyl (1r,2′S,4S)-6′-acetyl-4-(3-chloroanilino)-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylate

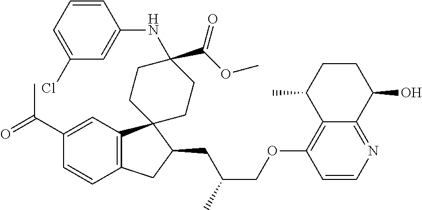 [SMILES: COC(=O)[C@]1(Nc2cccc(Cl)c2)CC[C@@]2(CC1)c1cc(C(C)=O)ccc1C[C@@H]2C[C@@H](C)COc1ccnc2c1[C@H](C)CC[C@H]2O]

Using General procedure 29 and Example 861A as the appropriate silyl derivative, Example 861B was obtained. ¹H NMR (500 MHz, DMSO-d₆) δ ppm: 8.25 (d, 1H), 7.91 (s, 1H), 7.82 (dd, 1H), 7.37 (d, 1H), 7.06 (t, 1H), 6.85 (d, 1H), 6.59 (dd, 1H), 6.57 (dd, 1H), 6.46 (dd, 1H), 6.35 (s, 1H), 4.93 (d, 1H), 4.44 (q, 1H), 3.92/3.86 (dd+dd, 2H), 3.66 (s, 3H), 3.09/2.61 (dd+dd, 2H), 3.01 (m, 1H), 2.57 (s, 3H), 2.52-1.19 (m, 8H), 2.22 (m, 1H), 2.08/1.44 (m+m, 2H), 2.01 (m, 1H), 1.94/1.72 (m+m, 2H), 1.47/1.35 (dd+dd, 2H), 1.05 (d, 3H), 1.00 (d, 3H). HRMS calculated for C₃₈H₄₅ClN₂O₅: 644.3017; found: 645.3082 (M+H).

### Example 861C methyl (1r,2′S,4S)-4-(3-chloroanilino)-6′-(1-hydroxyethyl)-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylate, diastereoisomer 1

and

### Example 862C methyl (1r,2′S,4S)-4-(3-chloroanilino)-6′-(1-hydroxyethyl)-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylate, diastereoisomer 2

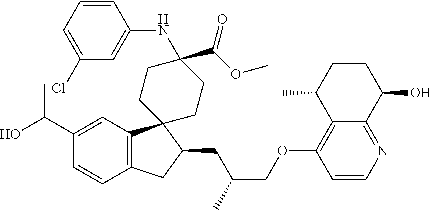 [SMILES: COC(=O)[C@]1(Nc2cccc(Cl)c2)CC[C@@]2(CC1)c1cc(C(C)O)ccc1C[C@@H]2C[C@@H](C)COc1ccnc2c1[C@H](C)CC[C@H]2O]

Using General procedure 36 and Example 861B as the appropriate acetyl derivative, a mixture of diastereoisomers was obtained. They were separated by chiral chromatography. Column: AD, 50×500 mm, 20 μm, Eluents: 8:32:60 MeCN/EtOH/Heptane+0.05% DEA. The diastereoisomer eluting earlier was collected as Example 861C. ¹H NMR (500 MHz, DMSO-d₆) δ ppm: 8.25 (d, 1H), 7.34 (br s, 1H), 7.13 (d, 1H), 7.11 (dd, 1H), 7.05 (t, 1H), 6.88 (t, 1H), 6.85 (d, 1H), 6.56 (dm, 1H), 6.46 (dm, 1H), 6.33 (s, 1H), 5.09 (d, 1H), 4.93 (d, 1H), 4.69 (m, 1H), 4.44 (m, 1H), 3.92/3.85 (dd+dd, 2H), 3.65 (s, 3H), 3.02 (m, 1H), 2.97/2.50 (dd+dd, 2H), 2.52-1.26 (m, 8H), 2.16 (m, 1H), 2.06/1.37 (m+m, 2H), 2.00 (m, 1H), 1.94/1.73 (m+m, 2H), 1.46/1.33 (m+m, 2H), 1.31 (d, 3H), 1.05 (d, 3H), 1.01 (d, 3H). HRMS calculated for C₃₈H₄₇ClN₂O₅: 646.3173; found: 647.3235 (M+H).

The diastereoisomer eluting later was collected as Example 862C. ¹H NMR (500 MHz, DMSO-d₆) δ ppm: 8.25 (d, 1H), 7.37 (br s, 1H), 7.13 (d, 1H), 7.09 (d, 1H), 7.05 (t, 1H), 6.88 (t, 1H), 6.85 (d, 1H), 6.56 (dm, 1H), 6.46 (dm, 1H), 6.33 (s, 1H), 5.09 (br, 1H), 4.93 (br, 1H), 4.68 (q, 1H), 4.44 (m, 1H), 3.93/3.85 (dd+dd, 2H), 3.65 (s, 3H), 3.02 (m, 1H), 2.97/2.50 (dd+dd, 2H), 2.52-1.26 (m, 8H), 2.15 (m, 1H), 2.06/1.37 (m+m, 2H), 2.00 (m, 1H), 1.94/1.73 (m+m, 2H), 1.46/1.33 (m+m, 2H), 1.30 (d, 3H), 1.05 (d, 3H), 1.01 (d, 3H). HRMS calculated for C₃₈H₄₇ClN₂O₅: 646.3173; found: 647.3233 (M+H).

### Example 861 (1r,2′S,4S)-4-(3-chloroanilino)-6′-(1-hydroxyethyl)-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid, diastereoisomer 1

and

### Example 862 (1r,2′S,4S)-4-(3-chloroanilino)-6′-(1-hydroxyethyl)-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid, diastereoisomer 2

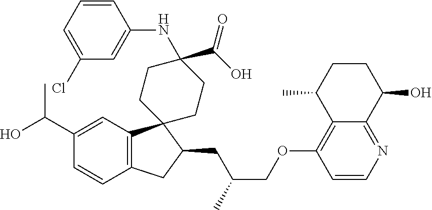 [SMILES: CC(O)c1ccc2c(c1)[C@]1(CC[C@@](Nc3cccc(Cl)c3)(C(=O)O)CC1)[C@@H](C[C@@H](C)COc1ccnc3c1[C@H](C)CC[C@H]3O)C2]

Using General procedure 33a and Example 861C as the appropriate ester derivative, Example 861 was obtained. ¹H NMR (500 MHz, DMSO-d₆) δ ppm: 12.68 (br s, 1H), 8.24 (d, 1H), 7.33 (d, 1H), 7.13 (d, 1H), 7.10 (dd, 1H), 7.04 (t, 1H), 6.85 (d, 1H), 6.63 (dd, 1H), 6.56 (dd, 1H), 6.54 (dd, 1H), 6.26 (br s, 1H), 5.08 (d, 1H), 4.93 (d, 1H), 4.68 (m, 1H), 4.44 (q, 1H), 3.92/3.85 (dd+dd, 2H), 3.02 (m, 1H), 2.97/2.50 (dd+dd, 2H), 2.50-1.32 (m, 8H), 2.18 (m, 1H), 2.06/1.38 (dd+dd, 2H), 1.99 (m, 1H), 1.96/1.72 (dd+dd, 2H), 1.45/1.35 (dd+dd, 2H), 1.30 (d, 3H), 1.05 (d, 3H), 1.01 (d, 3H). HRMS calculated for C₃₇H₄₅N₂O₅Cl: 632.3017; found: 633.3084 (M+H).

Using General procedure 33a and Example 862C as the appropriate ester derivative, Example 862 was obtained. ¹H NMR (500 MHz, DMSO-d₆) δ ppm: 12.66 (br s, 1H), 8.24 (d, 1H), 7.35 (d, 1H), 7.12 (d, 1H), 7.08 (dd, 1H), 7.04 (t, 1H), 6.85 (d, 1H), 6.62 (dd, 1H), 6.55 (dd, 1H), 6.54 (dd, 1H), 6.24 (br s, 1H), 5.08 (d, 1H), 4.93 (d, 1H), 4.67 (m, 1H), 4.44 (q, 1H), 3.93/3.85 (dd+dd, 2H), 3.02 (m, 1H), 2.97/2.50 (dd+dd, 2H), 2.50-1.31 (m, 8H), 2.16 (m, 1H), 2.06/1.38 (dd+dd, 2H), 2.00 (m, 1H), 1.94/1.73 (dd+dd, 2H), 1.47/1.35 (dd+dd, 2H), 1.29 (d, 3H), 1.05 (d, 3H), 1.01 (d, 3H). HRMS calculated for C₃₇H₄₅N₂O₅Cl: 632.3017; found: 633.3079 (M+H).

### Example 863 and Example 864

### Example 863A methyl (1r,2′S,4S)-6′-acetyl-4-(3-chloroanilino)-2′-[(2R)-3-{[(5R,8R)-8-fluoro-5-methyl-5,6,7,8-tetrahydroquinolin-4-yl]oxy}-2-methylpropyl]-2′,3′-dihydrospiro[cyclohexane-1,1′-indene]-4-carboxylate

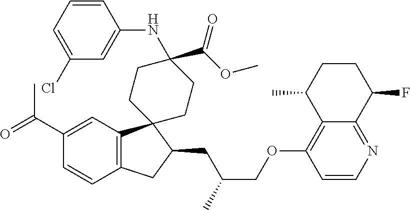 [SMILES: COC(=O)[C@]1(Nc2cccc(Cl)c2)CC[C@@]2(CC1)c1cc(C(C)=O)ccc1C[C@@H]2C[C@@H](C)COc1ccnc2c1[C@H](C)CC[C@H]2F]

Using General procedure 30a and Preparation 17a as the appropriate indane derivative and Example 772B as the appropriate alcohol, Example 863A was obtained. ¹H NMR (500 MHz, DMSO-d₆) δ ppm: 8.33 (d, 1H), 7.92 (d, 1H), 7.82 (dd, 1H), 7.36 (d, 1H), 7.05 (t, 1H), 6.99 (d, 1H), 6.59 (t, 1H), 6.57 (dm, 1H), 6.45 (dm, 1H), 6.36 (s, 1H), 5.36 (dm, 1H), 3.95/3.91 (dd+dd, 2H), 3.67 (s, 3H), 3.10 (m, 1H), 3.09/2.61 (dd+dd, 2H), 2.57 (s, 3H), 2.54-1.36 (m, 12H), 2.22 (m, 1H), 2.02 (m, 1H), 1.47/1.36 (m+m, 2H), 1.06 (d, 3H), 1.01 (d, 3H). HRMS calculated for C₃₈H₄₄ClFN₂O₄: 646.2974; found: 647.3041 (M+H).

### Example 863B methyl (1r,2′S,4S)-4-(3-chloroanilino)-2′-[(2R)-3-{[(5R,8R)-8-fluoro-5-methyl-5,6,7,8-tetrahydroquinolin-4-yl]oxy}-2-methylpropyl]-6′-(1-hydroxyethyl)-2′,3′-dihydrospiro[cyclohexane-1,1′-indene]-4-carboxylate, diastereoisomer 1

and

### Example 864B methyl (1r,2′S,4S)-4-(3-chloroanilino)-2′-[(2R)-3-{[(5R,8R)-8-fluoro-5-methyl-5,6,7,8-tetrahydroquinolin-4-yl]oxy}-2-methylpropyl]-6′-(1-hydroxyethyl)-2′,3′-dihydrospiro[cyclohexane-1,1′-indene]-4-carboxylate, diastereoisomer 2

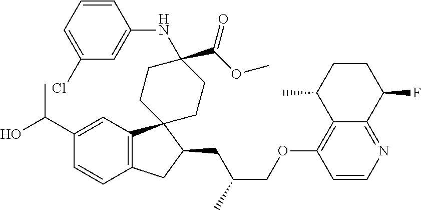 [SMILES: COC(=O)[C@]1(Nc2cccc(Cl)c2)CC[C@@]2(CC1)c1cc(C(C)O)ccc1C[C@@H]2C[C@@H](C)COc1ccnc2c1[C@H](C)CC[C@H]2F]

Using General procedure 36 and Example 863A as the appropriate acetyl derivative, a mixture of diastereoisomers was obtained. They were separated by chiral chromatography. Column: AD, 50×500 mm, 20 μm, Eluents: 50:50 EtOH/Heptane. The diastereoisomer eluting earlier was collected as Example 863B. ¹H NMR (500 MHz, DMSO-d₆) δ ppm: 8.33 (d, 1H), 7.35 (s, 1H), 7.12 (br s, 2H), 7.05 (t, 1H), 6.98 (d, 1H), 6.59 (t, 1H), 6.56 (dd, 1H), 6.45 (dd, 1H), 6.23 (s, 1H), 5.35 (dt, 1H), 5.09 (d, 1H), 4.70 (m, 1H), 3.95/3.90 (dd+dd, 2H), 3.65 (s, 3H), 3.11 (m, 1H), 2.97/2.50 (dd+dd, 2H), 2.48-1.38 (m, 8H), 2.15 (m, 1H), 2.15-2.06 (m, 2H), 2.02 (m, 1H), 1.96/1.53 (m+m, 2H), 1.46/1.35 (m+m, 2H), 1.31 (d, 3H), 1.05 (d, 3H), 1.02 (d, 3H). HRMS calculated for C₃₈H₄₆ClFN₂O₄: 648.3130; found: 649.3195 (M+H).

The diastereoisomer eluting later was collected as Example 864B. ¹H NMR (500 MHz, DMSO-d₆) δ ppm: 8.33 (d, 1H), 7.37 (br s, 1H), 7.12 (d, 1H), 7.09 (dd, 1H), 7.04 (t, 1H), 6.99 (d, 1H), 6.59 (t, 1H), 6.56 (dd, 1H), 6.45 (dd, 1H), 6.33 (s, 1H), 5.35 (dt, 1H), 5.09 (d, 1H), 4.68 (m, 1H), 3.95/3.90 (dd+dd, 2H), 3.65 (s, 3H), 3.11 (m, 1H), 2.97/2.50 (dd+dd, 2H), 2.49-1.33 (m, 8H), 2.16-2.05 (m, 2H), 2.15 (m, 1H), 2.00 (m, 1H), 1.96/1.53 (m+m, 2H), 1.48/1.35 (m+m, 2H), 1.31 (d, 3H), 1.05 (d, 3H), 1.02 (d, 3H). HRMS calculated for C₃₈H₄₆ClFN₂O₄: 648.3130; found: 649.3192 (M+H).

### Example 863 (1r,2′S,4S)-4-(3-chloroanilino)-2′-[(2R)-3-{[(5R,8R)-8-fluoro-5-methyl-5,6,7,8-tetrahydroquinolin-4-yl]oxy}-2-methylpropyl]-6′-(1-hydroxyethyl)-2′,3′-dihydrospiro[cyclohexane-1,1′-indene]-4-carboxylic acid, diastereoisomer 1

and

### Example 864 (1r,2′S,4S)-4-(3-chloroanilino)-2′-[(2R)-3-{[(5R,8R)-8-fluoro-5-methyl-5,6,7,8-tetrahydroquinolin-4-yl]oxy}-2-methylpropyl]-6′-(1-hydroxyethyl)-2′,3′-dihydrospiro[cyclohexane-1,1′-indene]-4-carboxylic acid, diastereoisomer 2

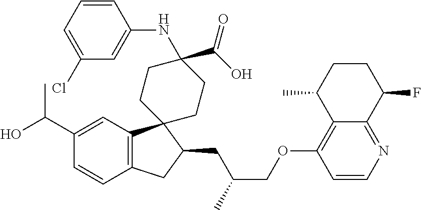 [SMILES: CC(O)c1ccc2c(c1)[C@]1(CC[C@@](Nc3cccc(Cl)c3)(C(=O)O)CC1)[C@@H](C[C@@H](C)COc1ccnc3c1[C@H](C)CC[C@H]3F)C2]

Using General procedure 33a and Example 863B as the appropriate ester derivative, Example 863 was obtained. ¹H NMR (500 MHz, DMSO-d₆) δ ppm: 12.65 (br s, 1H), 8.33 (d, 1H), 7.33 (br s, 1H), 7.12 (d, 1H), 7.10 (dd, 1H), 7.03 (t, 1H), 6.98 (d, 1H), 6.63 (t, 1H), 6.55 (dm, 1H), 6.53 (dm, 1H), 6.24 (br s, 1H), 5.35 (dm, 1H), 5.08 (d, 1H), 4.68 (m, 1H), 3.95/3.90 (dd+dd, 2H), 3.11 (m, 1H), 2.98/2.50 (dd+dd, 2H), 2.53-1.39 (m, 12H), 2.17 (m, 1H), 2.01 (m, 1H), 1.45/1.34 (m+m, 2H), 1.30 (d, 3H), 1.06 (d, 3H), 1.02 (d, 3H). HRMS calculated for C₃₇H₄₄ClFN₂O₄: 634.2974; found: 635.3032 (M+H).

Using General procedure 33a and Example 864B as the appropriate ester derivative, Example 864 was obtained. ¹H NMR (500 MHz, DMSO-d₆) δ ppm: 8.32 (d, 1H), 7.34 (br s, 1H), 7.11 (d, 1H), 7.08 (dd, 1H), 7.01 (t, 1H), 6.98 (d, 1H), 6.65 (t, 1H), 6.56 (dm, 1H), 6.49 (dm, 1H), 6.15 (br s, 1H), 5.35 (dm, 1H), 5.08 (d, 1H), 4.66 (m, 1H), 3.95/3.90 (dd+dd, 2H), 3.11 (m, 1H), 2.97/2.50 (dd+dd, 2H), 2.54-1.40 (m, 12H), 2.18 (m, 1H), 2.01 (m, 1H), 1.45/1.34 (m+m, 2H), 1.29 (d, 3H), 1.06 (d, 3H), 1.02 (s, 3H). HRMS calculated for C₃₇H₄₄ClFN₂O₄: 634.2974; found: 635.3041 (M+H).

### Example 865 (1r,2′S,4S)-4-(3-chloroanilino)-2′-[(2R)-3-{[(5R,8R)-5,8-dimethyl-5,6,7,8-tetrahydroquinolin-4-yl]oxy}-2-methylpropyl]-6′-(1-hydroxyethyl)-2′,3′-dihydrospiro[cyclohexane-1,1′-indene]-4-carboxylic acid

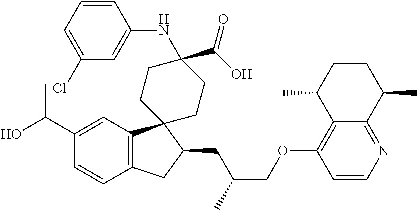 [SMILES: CC(O)c1ccc2c(c1)[C@]1(CC[C@@](Nc3cccc(Cl)c3)(C(=O)O)CC1)[C@@H](C[C@@H](C)COc1ccnc3c1[C@H](C)CC[C@H]3C)C2]

Using General procedure 30a and Preparation 17a as the appropriate indane derivative and Example 658G as the appropriate alcohol, an intermediate was obtained which was treated as described in General procedure 36. The obtained intermediate was hydrolyzed as described in General procedure 33a to obtain Example 865 as a mixture of diastereoisomers. ¹H NMR (500 MHz, DMSO-d₆) δ ppm: 12.68 (br s, 1H), 8.18 (d, 1H), 7.36/7.33 (d, 1H), 7.14-7.06 (m, 2H), 7.04 (t, 1H), 6.75 (d, 1H), 6.63 (t, 1H), 6.55 (dm, 1H), 6.53 (dm, 1H), 6.23 (br s, 1H), 5.08 (d, 1H), 4.68 (m, 1H), 3.90/3.83 (dd+dd, 2H), 3.03 (m, 1H), 2.96/2.49 (dd+dd, 2H), 2.86 (m, 1H), 2.54-1.27 (m, 16H), 1.30 (d, 3H), 1.17 (d, 3H), 1.05 (d, 3H), 1.03 (d, 3H). HRMS calculated for C₃₈H₄₇N₂O₄Cl: 630.3224; found: 631.3300 (M+H).

### Example 866

### Example 866A methyl (1s,4s)-2′-bromo-4-[(3-chlorophenyl)(trifluoroacetyl)amino]-6′-(2-hydroxypropan-2-yl)spiro[cyclohexane-1,1′-indene]-4-carboxylate

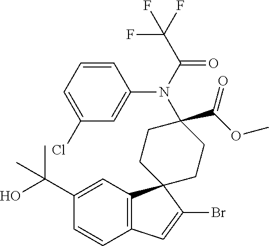 [SMILES: COC(=O)[C@]1(N(C(=O)C(F)(F)F)c2cccc(Cl)c2)CC[C@]2(CC1)C(Br)=Cc1ccc(C(C)(C)O)cc12]

Preparation 14aA (13.5 g, 23.1 mmol, 1.0 eq.) was dissolved in THE (230 mL) under N₂ atmosphere and cooled to 0° C. 3 M MeMgCl solution in THE (15.4 mL, 46.2 mmol, 2.0 eq.) was added and the mixture was stirred at 0° C. until no further conversion was observed. Then the reaction mixture was quenched with sat. aq. NH₄Cl solution, and extracted with DCM. The combined organic layers were dried over Na₂SO₄, filtered and the filtrate was concentrated under reduced pressure. The crude product was purified via flash chromatography using heptane and EtOAc as eluents, to obtain Example 866A (6.25 g, 10.4 mmol, 45%). ¹H NMR (500 MHz, DMSO-d₆) δ ppm: 7.70 (br s, 1H), 7.72-7.60 (m, 4H), 7.36 (dd, 1H), 7.23 (d, 1H), 6.98 (s, 1H), 5.04 (s, 1H), 3.84 (s, 3H), 2.61-2.50 (m, 1H), 2.45-2.36 (m, 2H), 2.33-2.25 (m, 1H), 2.79-2.70 (m, 1H), 1.60-1.30 (m, 3H), 1.44 (s, 6H). HRMS calculated for C₂₇H₂₆BrClF₃NO₄: 599.0686; found: 599.0634 (M+).

### Example 866B methyl (1s,4s)-2′-bromo-4-[(3-chlorophenyl)(trifluoroacetyl)amino]-6′-(prop-1-en-2-yl)spiro[cyclohexane-1,1′-indene]-4-carboxylate

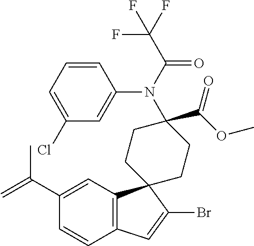 [SMILES: C=C(C)c1ccc2c(c1)[C@]1(CC[C@](C(=O)OC)(N(C(=O)C(F)(F)F)c3cccc(Cl)c3)CC1)C(Br)=C2]

Example 866A (4.15 g, 6.91 mmol, 1.0 eq.) and 2 M aq. HCl solution (69.1 mL, 138.2 mmol, 20.0 eq.) were stirred at reflux temperature until no further conversion was observed. The reaction mixture was diluted with water, the pH was set to 3-4 with sat. aq. NaHCO₃ solution, and then it was extracted with DCM. The combined organic layers were dried over Na₂SO₄, filtered and the filtrate was concentrated under reduced pressure. The crude product was purified via flash chromatography, using heptane and EtOAc as eluents to obtain Example 866B (1.61 g, 2.77 mmol, 40%). ¹H NMR (500 MHz, DMSO-d₆) δ ppm: 7.85-7.60 (m, 4H), 7.70 (d, 1H), 7.44 (dd, 1H), 7.31 (d, 1H), 7.03 (s, 1H), 5.44/5.14 (br s+m, 2H), 3.84 (s, 3H), 2.68-1.08 (m, 8H), 2.15 (s, 3H).

### Example 866C methyl (1s,4s)-2′-bromo-4-[(3-chlorophenyl)(trifluoroacetyl)amino]-6′-(2-methoxypropan-2-yl)spiro[cyclohexane-1,1′-indene]-4-carboxylate

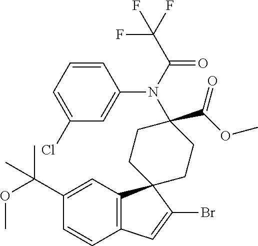 [SMILES: COC(=O)[C@]1(N(C(=O)C(F)(F)F)c2cccc(Cl)c2)CC[C@]2(CC1)C(Br)=Cc1ccc(C(C)(C)OC)cc12]

To a solution of Example 866B (1.61 g, 2.77 mmol, 1.0 eq.) in MeOH (27.7 mL) 4 M HCl solution in dioxane (5.53 mL, 22.1 mmol, 8.0 eq.) was added and the mixture was stirred at 70° C. until no further conversion was observed. Then it was cooled to rt and neutralized with sat. aq. NaHCO₃ solution, and then it was extracted with DCM. The combined organic layers were dried over Na₂SO₄, filtered and the filtrate was concentrated under reduced pressure. The crude product was purified via flash chromatography, using heptane and EtOAc as eluents to obtain Example 866C. ¹H NMR (500 MHz, DMSO-d₆) δ ppm: 7.84-7.60 (m, 4H), 7.61 (d, 1H), 7.29 (m, 1H), 7.29 (m, 1H), 7.01 (s, 1H), 3.85 (s, 3H), 3.00 (s, 3H), 2.62-1.26 (m, 8H), 1.47 (s, 6H). HRMS calculated for C₂₈H₂₈BrClF₃NO₄: 613.0842; found: 613.0846 (M+).

### Example 866D methyl (1r,4R)-4-[(3-chlorophenyl)(trifluoroacetyl)amino]-2′-{(2R)-3-[(4-methoxyphenyl)methoxy]-2-methylpropyl}-6′-(2-methoxypropan-2-yl)spiro[cyclohexane-1,1′-indene]-4-carboxylate

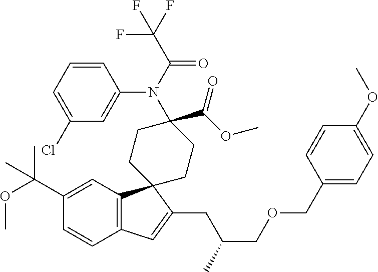 [SMILES: COc1ccc(COC[C@H](C)CC2=Cc3ccc(C(C)(C)OC)cc3[C@]23CC[C@](C(=O)OC)(N(C(=O)C(F)(F)F)c2cccc(Cl)c2)CC3)cc1]

Using General procedure 27b and Example 866C as the appropriate 2-bromo-indene derivative and Preparation 3d as the appropriate Zn reagent, Example 866D was obtained. ¹H NMR (500 MHz, DMSO-d₆) δ ppm: 7.92-7.48 (m, 4H), 7.64 (d, 1H), 7.25/7.24 (m, 2H), 7.22 (dd, 1H), 7.19 (d, 1H), 6.89 (m, 2H), 6.40/6.38 (s, 1H), 4.46-4.33 (d+d, 2H), 3.84 (s, 3H), 3.73 (s, 3H), 3.41-3.24 (m, 2H), 2.98 (s, 3H), 2.73-0.90 (m, 11H), 1.46 (s, 6H), 0.94/0.91 (d, 3H). HRMS calculated for C₄₀H₄₅ClF₃NO₆: 727.2888; found: 750.2759 (M+Na).

### Example 866E methyl (1r,4R)-4-[(3-chlorophenyl)(trifluoroacetyl)amino]-2′-[(2R)-3-hydroxy-2-methylpropyl]-6′-(2-methoxypropan-2-yl)spiro[cyclohexane-1,1′-indene]-4-carboxylate

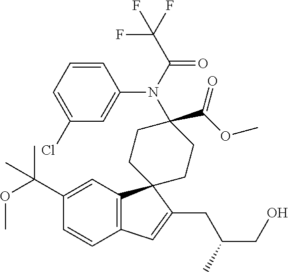 [SMILES: COC(=O)[C@]1(N(C(=O)C(F)(F)F)c2cccc(Cl)c2)CC[C@]2(CC1)C(C[C@@H](C)CO)=Cc1ccc(C(C)(C)OC)cc12]

Using General procedure 28a and Example 866D as the appropriate PMB derivative, Example 866E was obtained. ¹H NMR (500 MHz, DMSO-d₆) δ ppm: 7.93-7.58 (m, 4H), 7.65 (d, 1H), 7.22 (dd, 1H), 7.20 (d, 1H), 6.40 (s, 1H), 4.57/4.55 (t, 1H), 3.84 (s, 3H), 3.43-3.23 (m, 2H), 2.98 (s, 3H), 2.76-0.88 (m, 10H), 1.92 (m, 1H), 1.47 (s, 6H), 0.90/0.87 (d, 3H). HRMS calculated for C₃₂H₃₇ClF₃NO₅: 607.2313; found: 630.2208 (M+Na).

### Example 866F methyl (1r,2′S,4S)-4-[(3-chlorophenyl)(trifluoroacetyl)amino]-2′-[(2R)-3-hydroxy-2-methylpropyl]-6′-(2-methoxypropan-2-yl)-2′,3′-dihydrospiro[cyclohexane-1,1′-indene]-4-carboxylate

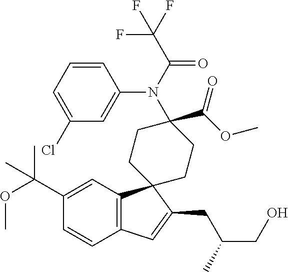

Using General procedure 19 and Example 866E as the appropriate indene, a mixture of diastereoisomers was obtained. They were separated via prep RP-HPLC using 25 mM aq. NH₄HCO₃ solution and MeCN as eluents. The diastereoisomer eluting earlier was collected as Example 866F. ¹H NMR (500 MHz, DMSO-d₆) δ ppm: 7.75-7.44 (m, 4H), 7.12-7.09 (m, 2H), 3.24-2.95 (m, 2H), 7.07/7.04 (br s, 1H), 4.38/4.35 (m, 1H), 3.79 (s, 3H), 2.94 (s, 3H), 2.93/2.46 (dd+dd, 2H), 2.49-0.55 (m, 10H), 2.22/2.17 (m, 1H), 1.47 (m, 1H), 1.42 (s, 6H), 0.74/0.71 (d, 3H). HRMS calculated for C₃₂H₃₉ClF₃NO₅: 609.2469; found: 632.2356 (M+Na).

### Example 866 (1r,2′S,4S)-4-(3-chloroanilino)-6′-(2-methoxypropan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

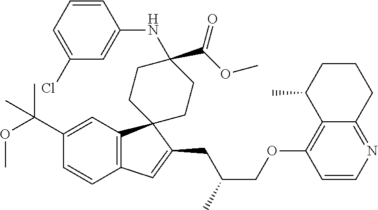

Using General procedure 32 and Example 866F as the appropriate indane and Preparation 2a1 as the appropriate alcohol, Example 866 was obtained. ¹H NMR (500 MHz, DMSO-d₆) δ ppm: 8.17 (d, 1H), 7.45 (br s, 1H), 7.16 (d, 1H), 7.14 (dd, 1H), 7.04 (t, 1H), 6.82 (d, 1H), 6.59 (t, 1H), 6.54 (dm, 1H), 6.52 (dm, 1H), 3.93/3.88 (dd+dd, 2H), 3.05 (m, 1H), 2.98/2.51 (dd+dd, 2H), 2.97 (s, 3H), 2.77/2.66 (dm+m, 2H), 2.52-1.30 (m, 16H), 1.44/1.42 (s+s, 6H), 1.05 (d, 3H), 1.05 (d, 3H). HRMS calculated for C₃₉H₄₉N₂O₄Cl: 644.3381; found: 645.3440 (M+H).

### Example 867 (1r,2′S,4S)-4-(3-chloroanilino)-6′-formyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

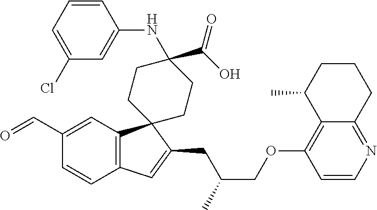

Using General procedure 33a and Preparation 13b as the appropriate indane, Example 867 was obtained as a white solid. LRMS calculated for C₃₆H₄₁ClN₂O₄: 600; found: 601 (M+H). ¹H NMR (400 MHz, DMSO-d₆) δ ppm: 9.98 (s, 1H), 8.15 (d, J=5.6 Hz, 1H), 7.86-7.82 (m, 1H), 7.75 (dd, J=7.6, 1.3 Hz, 1H), 7.46 (d, J=7.6 Hz, 1H), 7.04 (t, J=8.1 Hz, 1H), 6.77 (d, J=5.6 Hz, 1H), 6.65 (t, J=2.1 Hz, 1H), 6.59-6.51 (m, 2H), 3.94-3.82 (m, 2H), 3.13 (dd, J=16.7, 7.0 Hz, 1H), 3.08-2.98 (m, 1H), 2.81-2.72 (m, 1H), 2.71-2.59 (m, 2H), 2.49-2.39 (m, 1H), 2.32-2.21 (m, 1H), 2.21-2.10 (m, 1H), 2.07-1.31 (m, 13H), 1.06 (d, J=6.6 Hz, 3H), 1.01 (d, J=6.9 Hz, 3H).

### Example 868

### Example 868A methyl (1r,4R)-4-(3-chloroanilino)-6′-formyl-2′-[(2R)-2-methyl-3-{[(5R)-5-methyl-5,6,7,8-tetrahydroquinolin-4-yl]oxy}propyl]spiro[cyclohexane-1,1′-indene]-4-carboxylate

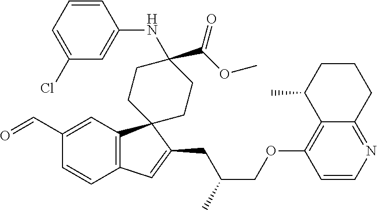

Using General procedure 30a and Preparation 13aF as the appropriate indene and Preparation 2a1 as the appropriate alcohol, an intermediate was obtained which was treated as described in General procedure 9 to obtain Example 868A. ¹H NMR (500 MHz, DMSO-d₆) δ ppm: 9.99 (s, 1H), 8.14 (d, 1H), 8.13 (s, 1H), 7.85 (dd, 1H), 7.52 (d, 1H), 7.09 (t, 1H), 6.77 (d, 1H), 6.72 (s, 1H), 6.64 (t, 1H), 6.60 (dd, 1H), 6.48 (s, 1H), 6.47 (dd, 1H), 4.03/3.95 (dd+dd, 2H), 3.71 (s, 3H), 3.06 (m, 1H), 2.76/2.64 (m+m, 2H), 2.58-0.86 (m, 8H), 2.55/2.27 (dd+dd, 2H), 2.48 (m, 1H), 1.78/1.70 (m+m, 2H), 1.66/1.54 (m+m, 2H), 1.11 (d, 3H), 1.10 (d, 3H). HRMS calculated for C₃₇H₄₁ClN₂O₄: 612.2755; found: 613.2822 (M+H).

### Example 868B methyl (1r,4R)-4-(3-chloroanilino)-6′-(hydroxymethyl)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylate

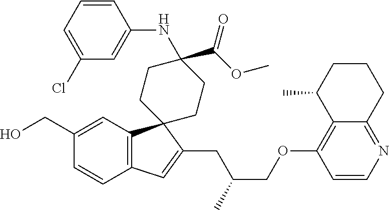 [SMILES: COC(=O)[C@]1(Nc2cccc(Cl)c2)CC[C@]2(CC1)C(C[C@@H](C)COc1ccnc3c1[C@H](C)CCC3)=Cc1ccc(CO)cc12]

Using General procedure 36 and Example 868A as the appropriate formyl derivative, Example 868B was obtained. ¹H NMR (500 MHz, DMSO-d₆) δ ppm: 8.13 (d, 1H), 7.65 (br s, 1H), 7.22 (d, 1H), 7.17 (dd, 1H), 7.07 (t, 1H), 6.76 (d, 1H), 6.64 (t, 1H), 6.59 (dd, 1H), 6.52 (s, 1H), 6.47 (dd, 1H), 6.44 (s, 1H), 5.14 (t, 1H), 4.50 (d, 2H), 4.01/3.93 (m+m, 2H), 3.68 (s, 3H), 3.06 (m, 1H), 2.75/2.63 (m+m, 2H), 2.45-0.88 (m, 14H), 2.42 (m, 1H), 1.10 (d, 3H), 1.10 (d, 3H). HRMS calculated for C₃₇H₄₃ClN₂O₄: 614.2911; found: 615.2983 (M+H).

### Example 868 (1r,4R)-4-(3-chloroanilino)-6′-(hydroxymethyl)-2′-[(2R)-2-methyl-3-{[(5R)-5-methyl-5,6,7,8-tetrahydroquinolin-4-yl]oxy}propyl]spiro[cyclohexane-1,1′-indene]-4-carboxylic acid

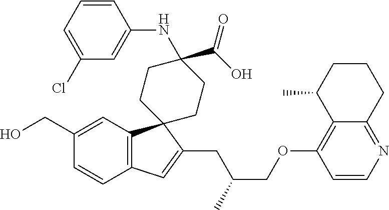 [SMILES: C[C@@H](COc1ccnc2c1[C@H](C)CCC2)CC1=Cc2ccc(CO)cc2[C@]12CC[C@@](Nc1cccc(Cl)c1)(C(=O)O)CC2]

Using General procedure 33a and Example 868B as the appropriate ester derivative, Example 868 was obtained. ¹H NMR (500 MHz, DMSO-d₆) δ ppm: 12.80 (br s, 1H), 8.13 (d, 1H), 7.64 (s, 1H), 7.22 (d, 1H), 7.16 (dd, 1H), 7.07 (t, 1H), 6.76 (d, 1H), 6.64 (t, 1H), 6.56 (m, 2H), 6.51 (s, 1H), 6.33 (br s, 1H), 5.12 (t, 1H), 4.49 (d, 2H), 4.02/3.93 (dd+dd, 2H), 3.06 (m, 1H), 2.75/2.63 (m+m, 2H), 2.46/2.20 (dd+dd, 2H), 2.46-0.80 (m, 8H), 2.42 (m, 1H), 1.79/1.70 (m+m, 2H), 1.60/1.55 (m+m, 2H), 1.11 (d, 3H), 1.10 (d, 3H). HRMS calculated for C₃₆H₄₁N₂O₄Cl: 600.2755; found: 601.2813 (M+H).

### Example 869 (1r,2′R,4R)-4-(3-chloroanilino)-6′-(hydroxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

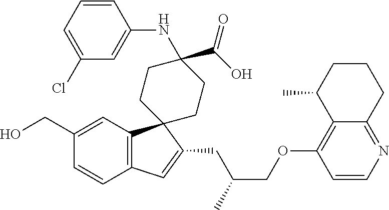

and

### Example 871 (1r,2′R,4R)-4-(3-chloroanilino)-2′-[(2R)-2-methyl-3-{[(5R)-5-methyl-5,6,7,8-tetrahydroquinolin-4-yl]oxy}propyl]-6′-{[(oxolan-2-yl)oxy]methyl}-2′,3′-dihydrospiro[cyclohexane-1,1′-indene]-4-carboxylic acid

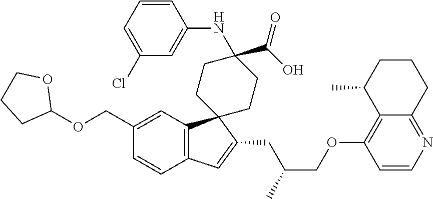

Preparation 13d (as the appropriate ester derivative) was hydrolyzed according to General procedure 33a. During extraction inhibitor-free THE was used which gave rise to a side product. They were separated and purified via prep RP-HPLC using 25 mM aq. NH₄HCO₃ solution and MeCN as eluents. The product eluting earlier was collected as Example 869. ¹H NMR (500 MHz, DMSO-d₆) δ ppm: 12.66 (br s, 1H), 8.13 (d, 1H), 7.33 (d, 1H), 7.13 (d, 1H), 7.06 (dd, 1H), 7.04 (t, 1H), 6.78 (d, 1H), 6.62 (t, 1H), 6.54 (dm, 1H), 6.51 (dm, 1H), 6.12 (br s, 1H), 5.11 (t, 1H), 4.45 (d, 2H), 4.01/3.87 (dd+dd, 2H), 3.01 (m, 1H), 2.98/2.55 (dd+dd, 2H), 2.73/2.60 (m+m, 2H), 2.46-1.30 (m, 12H), 2.11 (m, 1H), 2.07 (m, 1H), 1.69/1.20 (m+m, 2H), 1.09 (d, 3H), 1.09 (d, 3H). HRMS calculated for C₃₆H₄₃N₂O₄Cl: 602.2911; found: 603.2980 (M+H).

The product eluting later was collected as Example 871, isolated as a mixture of diastereoisomers. ¹H NMR (500 MHz, DMSO-d₆) δ ppm: 12.66 (br s, 1H), 8.13 (d, 1H), 7.34 (d, 1H), 7.15 (d, 1H), 7.06 (dd, 1H), 7.03 (t, 1H), 6.77 (d, 1H), 6.61 (t, 1H), 6.54 (dm, 1H), 6.51 (dm, 1H), 6.13 (br s, 1H), 5.16 (dd, 1H), 4.56/4.39 (d+d, 2H), 4.00/3.87 (dd+dd, 2H), 3.80 (m, 2H), 3.02 (m, 1H), 2.98/2.57 (dd+dd, 2H), 2.73/2.60 (m+m, 2H), 2.46-1.28 (m, 16H), 2.10 (m, 1H), 2.07 (m, 1H), 1.70/1.23 (m+m, 2H), 1.10 (d, 3H), 1.09 (d, 3H). HRMS calculated for C₄₀H₄₉N₂O₅Cl: 672.3330; found: 673.3400 (M+H).

### Example 870 (1r,2′S,4S)-4-(3-chloroanilino)-6′-(hydroxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

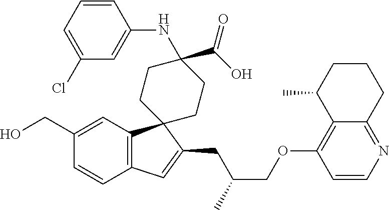

and

### Example 872 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-{[(oxolan-2-yl)oxy]methyl}-2′,3′-dihydrospiro[cyclohexane-1,1′-indene]-4-carboxylic acid

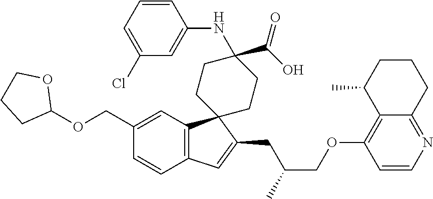

Preparation 13c (as the appropriate ester derivative) was hydrolyzed according to General procedure 33a. During extraction inhibitor-free THE was used which gave rise to a side product. They were separated and purified via prep RP-HPLC using 25 mM aq. NH₄HCO₃ solution and MeCN as eluents. The product eluting earlier was collected as Example 870. ¹H NMR (500 MHz, DMSO-d₆) δ ppm: 12.66 (br s, 1H), 8.14 (d, 1H), 7.31 (d, 1H), 7.13 (d, 1H), 7.07 (dd, 1H), 7.04 (t, 1H), 6.76 (d, 1H), 6.63 (t, 1H), 6.55 (dm, 1H), 6.54 (dm, 1H), 6.23 (br s, 1H), 5.12 (t, 1H), 4.45 (d, 2H), 3.90/3.84 (dd+dd, 2H), 3.04 (m, 1H), 2.98/2.50 (dd+dd, 2H), 2.76/2.65 (m+m, 2H), 2.50-1.35 (m, 12H), 2.16 (m, 1H), 1.99 (m, 1H), 1.45/1.33 (m+m, 2H), 1.05 (d, 3H), 1.04 (d, 3H). HRMS calculated for C₃₆H₄₃N₂O₄Cl: 602.2911; found: 603.2982 (M+H).

The product eluting earlier was collected as Example 872, isolated as a mixture of diastereoisomers. ¹H NMR (500 MHz, DMSO-d₆) δ ppm: 12.67 (br s, 1H), 8.14 (d, 1H), 7.31 (d, 1H), 7.15 (d, 1H), 7.06 (dd, 1H), 7.04 (t, 1H), 6.76 (d, 1H), 6.62 (t, 1H), 6.55 (dm, 1H), 6.53 (dm, 1H), 6.24 (br s, 1H), 5.17 (dd, 1H), 4.57/4.39 (d+d, 2H), 3.89/3.85 (dd+dd, 2H), 3.80 (m, 2H), 3.04 (m, 1H), 2.98/2.51 (dd+dd, 2H), 2.76/2.65 (m+m, 2H), 2.50-1.32 (m, 12H), 2.15 (m, 1H), 1.99 (m, 1H), 1.90/1.78 (m+m, 2H), 1.88/1.82 (m+m, 2H), 1.46/1.34 (m+m, 2H), 1.04 (d, 3H), 1.03 (d, 3H). HRMS calculated for C₄₀H₄₉N₂O₅Cl: 672.3330; found: 673.3408 (M+H).

### Example 873

### Example 873A methyl (1s,4s)-6′-acetyl-2′-bromo-4-[(3-chlorophenyl)(trifluoroacetyl)amino]-5′-fluorospiro[cyclohexane-1,1′-indene]-4-carboxylate

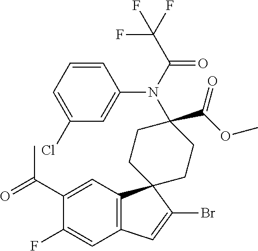 [SMILES: COC(=O)[C@]1(N(C(=O)C(F)(F)F)c2cccc(Cl)c2)CC[C@]2(CC1)C(Br)=Cc1cc(F)c(C(C)=O)cc12]

Using General procedure 22 and Example 737A (140 mg, 0.30 mmol, 1.0 eq) as the appropriate 2-bromo-indene derivative, an intermediate was obtained, which was treated as described in General procedure 23 to obtain Example 873A. ¹H NMR (500 MHz, DMSO-d₆) δ ppm: 7.96 (d, 1H), 7.82 (br s, 1H), 7.70-7.61 (m, 3H), 7.32 (d, 1H), 7.12 (s, 1H), 3.86 (s, 3H), 2.56 (s, 3H), 2.47-1.35 (m, 8H). HRMS calculated for C₂₆H₂₁BrClF₄NO₄: 601.0278; found: 602.0339 (M+H).

### Example 873B methyl (1r,4R)-6′-acetyl-4-[(3-chlorophenyl)(trifluoroacetyl)amino]-5′-fluoro-2′-{(2R)-3-[(4-methoxyphenyl)methoxy]-2-methylpropyl}spiro[cyclohexane-1,1′-indene]-4-carboxylate

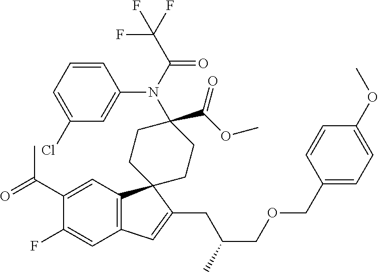 [SMILES: COc1ccc(COC[C@H](C)CC2=Cc3cc(F)c(C(C)=O)cc3[C@]23CC[C@](C(=O)OC)(N(C(=O)C(F)(F)F)c2cccc(Cl)c2)CC3)cc1]

Using General procedure 27b and Example 873A as the appropriate 2-bromo-indene derivative and Preparation 3d as the appropriate Zn Reagent, Example 873B was obtained. ¹H NMR (500 MHz, DMSO-d₆) δ ppm: 8.01 (d, 1H), 7.92-7.48 (m, 4H), 7.25/7.23 (m, 2H), 7.19 (d, 1H), 6.89 (m, 2H), 6.53/6.51 (br s, 1H), 4.46-4.33 (d+d, 2H), 3.85 (s, 3H), 3.73 (s, 3H), 3.40-3.24 (m, 2H), 2.62-0.94 (m, 11H), 2.57/2.56 (s, 3H), 0.94/0.91 (d, 3H). HRMS calculated for C₃₈H₃₈ClF₄NO₆: 715.2324; found: 716.2360 (M+H).

### Example 873 (1r,4R)-6′-acetyl-4-(3-chloroanilino)-5′-fluoro-2′-[(2R)-2-methyl-3-{[(5S)-5-methyl-5,6,7,8-tetrahydroquinolin-4-yl]oxy}propyl]spiro[cyclohexane-1,1′-indene]-4-carboxylic acid

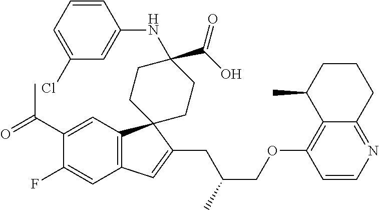 [SMILES: CC(=O)c1cc2c(cc1F)C=C(C[C@@H](C)COc1ccnc3c1[C@@H](C)CCC3)[C@]21CC[C@](Nc2cccc(Cl)c2)(C(=O)O)CC1]

Using General procedure 28b and Example 873B as the appropriate PMB derivative, an intermediate was obtained, which was treated as described in General procedure 32, using Preparation 2a2 as the appropriate alcohol to obtain Example 873. ¹H NMR (500 MHz, DMSO-d₆) δ ppm: 8.15 (d, 1H), 8.13 (d, 1H), 7.25 (d, 1H), 7.04 (t, 1H), 6.77 (d, 1H), 6.64 (br s, 1H), 6.60 (s, 1H), 6.53 (m, 2H), 6.26 (br s, 1H), 3.98 (m, 2H), 3.05 (m, 1H), 2.76/2.65 (d+m, 2H), 2.57 (d, 3H), 2.52/2.28 (dd+dd, 2H), 2.44 (m, 1H), 2.39-0.82 (m, 8H), 1.80/1.74 (m+m, 2H), 1.65/1.60 (m+m, 2H), 1.12 (d, 3H), 1.10 (d, 3H). HRMS calculated for C₃₇H₄₀N₂04FCl: 630.2661; found: 631.2735 (M+H).

### Example 874 (1r,2′S,4S)-4-(3-chloroanilino)-6′-[(1S,2S)-2-(methoxymethyl)cyclopropyl]-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

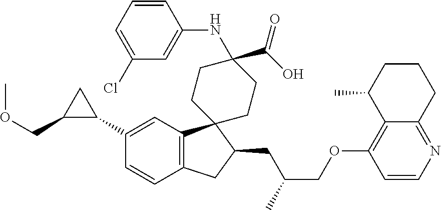 [SMILES: COC[C@H]1C[C@@H]1c1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

### Example 875 (1r,2′S,4S)-4-(3-chloroanilino)-6′-[(1R,2R)-2-(methoxymethyl)cyclopropyl]-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

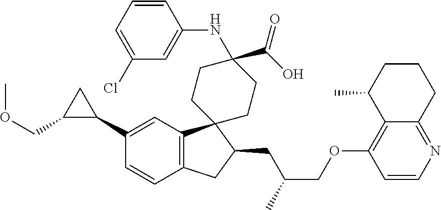 [SMILES: COC[C@@H]1C[C@H]1c1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

To a solution of Preparation 16a (400 mg, 0.48 mmol, 1.0 eq.) in dry toluene (4.8 mL) in a microwave tube (±)-trans-2-(methoxymethyl)cyclopropylboronic acid (94 mg, 0.722 mmol, 1.5 eq), Pd(PPh₃)₄ (111 mg, 0.096 mmol, 0.2 eq.), Cs₂CO₃ (157 mg, 0.48 mmol, 1.0 eq.) and KF×2H₂O (121 mg, 1.59 mmol, 3.3 eq.) were added and the mixture was sparged with N₂, then sealed and was irradiated in a microwave reactor at 120° C. until no further conversion was observed. Then it was concentrated under reduced pressure and purified via prep RP-HPLC using 25 mM aq. NH₄HCO₃ solution and MeCN as eluents to obtain an intermediate, which was hydrolyzed according to General procedure 33a to obtain a mixture of diastereoisomers. They were separated via SFC chromatography. The diastereoisomer eluting earlier was collected as Example 874. ¹H NMR (500 MHz, DMSO-d₆) δ ppm: 8.14 (d, 1H), 7.06 (m, 1H), 7.05 (d, 1H), 7.02 (t, 1H), 6.84 (dd, 1H), 6.76 (d, 1H), 6.61 (t, 1H), 6.54 (dd, 1H), 6.51 (m, 1H), 6.20 (br s, 1H), 3.90/3.83 (dd+dd, 2H), 3.41/3.24 (dd+dd, 2H), 3.25 (s, 3H), 3.04 (m, 1H), 2.94/2.46 (dd+dd, 2H), 2.76/2.65 (dm+m, 2H), 2.48-1.27 (m, 14H), 2.13 (m, 1H), 1.97 (m, 1H), 1.79 (m, 1H), 1.25 (m, 1H), 1.04 (d, 3H), 1.04 (d, 3H), 0.84 (m, 2H). HRMS calculated for C₄₀H₄₉N₂O₄Cl: 656.3381; found: 657.3462 (M+H).

The diastereoisomer eluting later was collected as Example 875. ¹H NMR (500 MHz, DMSO-d₆) δ ppm: 8.14 (d, 1H), 7.06 (s, 1H), 7.05 (d, 1H), 7.04 (t, 1H), 6.84 (dd, 1H), 6.76 (d, 1H), 6.60 (t, 1H), 6.53 (m, 2H), 6.22 (br s, 1H), 3.90/3.84 (d/d+d/d, 2H), 3.39/3.24 (m+m, 2H), 3.25 (s, 3H), 3.04 (m, 1H), 2.93/2.46 (dd+dd, 2H), 2.76/2.65 (dm+m, 2H), 2.49-1.27 (m, 14H), 2.12 (m, 1H), 1.98 (m, 1H), 1.80 (m, 1H), 1.26 (m, 1H), 1.05 (d, 3H), 1.03 (d, 3H), 0.85 (m, 2H). HRMS calculated for C₄₀H₄₉N₂O₄Cl: 656.3381; found: 657.3451 (M+H).

### Example 876 (1r,2′S,4S)-4-(3-chloroanilino)-6′-(2-methoxyphenyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

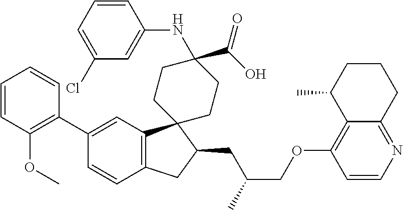 [SMILES: COc1ccccc1-c1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 34 and (2-methoxyphenyl)boronic acid as the appropriate boronic acid, an intermediate was obtained, which was hydrolyzed as described in General procedure 33a to obtain Example 876. ¹H NMR (500 MHz, DMSO-d₆) δ ppm: 12.67 (br s, 1H), 8.15 (d, 1H), 7.58 (s, 1H), 7.32 (m, 1H), 7.27 (dm, 1H), 7.23 (s, 1H), 7.23 (s, 1H), 7.10 (dm, 1H), 7.04 (t, 1H), 7.02 (m, 1H), 6.78 (d, 1H), 6.61 (t, 1H), 6.53 (m, 2H), 6.26 (br s, 1H), 3.94/3.87 (dd+dd, 2H), 3.77 (s, 3H), 3.08 (m, 1H), 3.02/2.55 (dd+dd, 2H), 2.77/2.66 (m+m, 2H), 2.50-1.29 (m, 12H), 2.15 (m, 1H), 2.02 (m, 1H), 1.55/1.39 (m+m, 2H), 1.08 (d, 3H), 1.06 (d, 3H). HRMS calculated for C₄₂H₄₇N₂O₄Cl: 678.3224; found: 679.3279 (M+H).

### Example 877 (1r,2′S,4S)-4-(3-chloroanilino)-6′-(3-methoxyphenyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

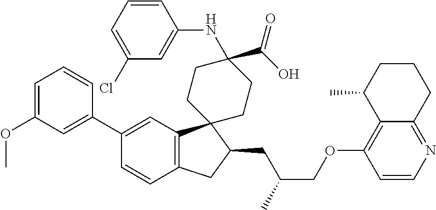 [SMILES: COc1cccc(-c2ccc3c(c2)[C@]2(CC[C@@](Nc4cccc(Cl)c4)(C(=O)O)CC2)[C@@H](C[C@@H](C)COc2ccnc4c2[C@H](C)CCC4)C3)c1]

Using General procedure 34 and (3-methoxyphenyl)boronic acid as the appropriate boronic acid, an intermediate was obtained, which was hydrolyzed as described in General procedure 33a to obtain Example 877. ¹H NMR (500 MHz, DMSO-d₆) δ ppm: 12.7 (br s, 1H), 8.15 (d, 1H), 7.61 (s, 1H), 7.44 (dd, 1H), 7.37 (t, 1H), 7.29 (d, 1H), 7.16 (dd, 1H), 7.11 (t, 1H), 7.04 (t, 1H), 6.92 (dd, 1H), 6.78 (d, 1H), 6.60 (t, 1H), 6.54 (dd, 1H), 6.53 (dd, 1H), 6.27 (br s, 1H), 3.93/3.87 (dd+dd, 2H), 3.82 (s, 3H), 3.06 (m, 1H), 3.05/2.57 (dd+dd, 2H), 2.76/2.65 (m+m, 2H), 2.54-1.39 (m, 8H), 2.20 (m, 1H), 2.02 (m, 1H), 1.80/1.74 (m+m, 2H), 1.69/1.62 (m+m, 2H), 1.53/1.38 (t+t, 2H), 1.07 (d, 3H), 1.06 (d, 3H). HRMS calculated for C₄₂H₄₇N₂O₄Cl: 678.3224; found: 679.3276 (M+H).

### Example 878 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-(thiophen-3-yl)-2′,3′-dihydrospiro[cyclohexane-1,1′-indene]-4-carboxylic acid

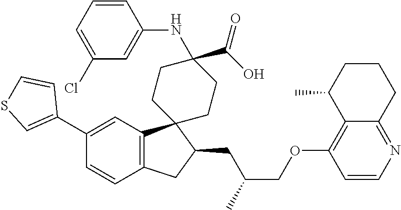 [SMILES: C[C@@H](COc1ccnc2c1[C@H](C)CCC2)C[C@H]1Cc2ccc(-c3ccsc3)cc2[C@]12CC[C@@](Nc1cccc(Cl)c1)(C(=O)O)CC2]

To an oven-dried microwave vial was added Preparation 16a (52 mg, 0.06 mmol, 1 eq), thiophen-3-ylboronic acid (12 mg, 0.09 mmol, 1.5 eq), K₂CO₃ (26 mg, 0.19 mmol, 3 eq) in THE (2 mL) and water (0.2 mL). The mixture was sparged with N₂ before the addition of Pd(dppf)Cl₂×DCM (5 mg, 0.01 mmol, 0.1 eq) and the mixture was heated at 110° C. for 1 h under microwave irradiation. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-10% MeOH in DCM afforded an intermediate, which was hydrolyzed as described in General procedure 33a to obtain Example 878 as a white powder. LRMS calculated for C₃₉H₄₃ClN₂O₃S: 654; found: 655 (M+H). ¹H NMR (400 MHz, DMSO-d₆) δ ppm: 8.15 (d, J=5.7 Hz, 1H), 7.74 (dd, J=2.9, 1.4 Hz, 1H), 7.67-7.62 (m, 2H), 7.52-7.46 (m, 2H), 7.25 (d, J=7.8 Hz, 1H), 7.06 (t, J=8.1 Hz, 1H), 6.79 (d, J=5.7 Hz, 1H), 6.62 (t, J=2.1 Hz, 1H), 6.59-6.51 (m, 2H), 3.97-3.83 (m, 2H), 3.11-2.97 (m, 2H), 2.81-2.72 (m, 1H), 2.72-2.50 (m, 3H), 2.27-2.15 (m, 2H), 2.08-1.32 (m, 13H), 1.11-1.01 (m, 6H).

### Example 879 (1r,2′R,4R)-4-(3-chloroanilino)-2′-[(2R)-2-methyl-3-{[(5R)-5-methyl-5,6,7,8-tetrahydroquinolin-4-yl]oxy}propyl]-6′-(thiophen-3-yl)-2′,3′-dihydrospiro[cyclohexane-1,1′-indene]-4-carboxylic acid

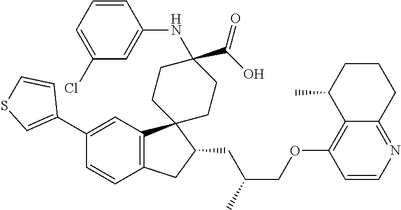 [SMILES: C[C@@H](COc1ccnc2c1[C@H](C)CCC2)C[C@@H]1Cc2ccc(-c3ccsc3)cc2[C@]12CC[C@@](Nc1cccc(Cl)c1)(C(=O)O)CC2]

To an oven-dried microwave vial was added Preparation 16b (113 mg, 0.14 mmol, 1 eq), thiophen-3-ylboronic acid (26 mg, 0.2 mmol, 1.5 eq), K₂CO₃ (56 mg, 0.41 mmol, 3 eq) in THE (2 mL) and water (0.2 mL). The mixture was sparged with N₂ before the addition of Pd(dppf)Cl₂×DCM (11 mg, 0.01 mmol, 0.1 eq) and the mixture was heated at 110° C. for 2 h under microwave irradiation. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-10% MeOH in DCM afforded an intermediate, which was hydrolyzed as described in General procedure 33a to obtain Example 879 as a white powder. LRMS calculated for C₃₉H₄₃ClN₂O₃S: 654; found: 655 (M+H). ¹H NMR (400 MHz, DMSO-d₆) δ ppm: 12.70 (br s, 1H), 8.22 (d, J=5.7 Hz, 1H), 7.73 (dd, J=2.9, 1.3 Hz, 1H), 7.69-7.63 (m, 2H), 7.52-7.45 (m, 2H), 7.25 (d, J=7.7 Hz, 1H), 7.06 (t, J=8.1 Hz, 1H), 6.90 (d, J=5.7 Hz, 1H), 6.61 (t, J=2.1 Hz, 1H), 6.58-6.54 (m, 1H), 6.53-6.48 (m, 1H), 6.17 (br s, 1H), 4.11-4.03 (m, 1H), 4.00-3.91 (m, 1H), 3.09-2.99 (m, 2H), 2.82-2.72 (m, 1H), 2.67-2.43 (m, 3H), 2.23-1.98 (m, 4H), 1.94-1.62 (m, 6H), 1.60-1.37 (m, 4H), 1.33-1.22 (m, 1H), 1.15-1.09 (m, 6H).

### Example 880 (1r,2′R,4R)-4-(3-chloroanilino)-2′-[(2R)-2-methyl-3-{[(5R)-5-methyl-5,6,7,8-tetrahydroquinolin-4-yl]oxy}propyl]-6′-(1H-pyrrol-2-yl)-2′,3′-dihydrospiro[cyclohexane-1,1′-indene]-4-carboxylic acid

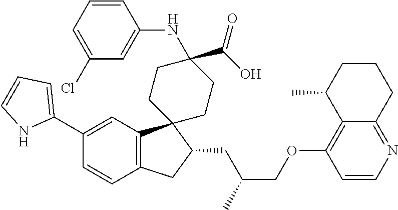 [SMILES: C[C@@H](COc1ccnc2c1[C@H](C)CCC2)C[C@@H]1Cc2ccc(-c3ccc[nH]3)cc2[C@]12CC[C@@](Nc1cccc(Cl)c1)(C(=O)O)CC2]

To an oven-dried microwave vial was added Preparation 16b (120 mg, 0.14 mmol, 1 eq), [1-(tert-butoxycarbonyl)-1H-pyrrol-2-yl]boronic acid (46 mg, 0.22 mmol, 1.5 eq), K₂CO₃ (60 mg, 0.43 mmol, 3 eq) in THE (3 mL) and water (0.3 mL). The mixture was sparged with N₂ before the addition of Pd(dppf)Cl₂×DCM (11 mg, 0.01 mmol, 0.1 eq) and the mixture was heated at 110° C. for 1 h under microwave irradiation. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-100% EtOAc in heptane afforded an intermediate, which was hydrolyzed as described in General procedure 33a to obtain Example 880 as a white powder. LRMS calculated for C₃₉H₄₄ClN₃O₃: 637; found: 638 (M+H). ¹H NMR (400 MHz, DMSO-d₆) δ ppm: 12.64 (br s, 1H), 11.28-11.22 (m, 1H), 8.20 (d, J=5.7 Hz, 1H), 7.63-7.58 (m, 1H), 7.39 (dd, J=7.8, 1.5 Hz, 1H), 7.18 (d, J=7.8 Hz, 1H), 7.05 (t, J=8.1 Hz, 1H), 6.87 (d, J=5.7 Hz, 1H), 6.84-6.80 (m, 1H), 6.63 (t, J=2.1 Hz, 1H), 6.58-6.50 (m, 2H), 6.39-6.34 (m, 1H), 6.14 (br s, 1H), 6.12-6.07 (m, 1H), 4.06 (dd, J=9.7, 4.3 Hz, 1H), 3.93 (dd, J=9.7, 5.4 Hz, 1H), 3.09-2.95 (m, 2H), 2.81-2.72 (m, 1H), 2.68-2.50 (m, 3H), 2.24-2.05 (m, 3H), 2.05-1.95 (m, 1H), 1.93-1.63 (m, 6H), 1.61-1.38 (m, 4H), 1.29-1.19 (m, 1H), 1.15-1.08 (m, 6H).

### Example 881 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-(1-methyl-1H-pyrrol-2-yl)-2′,3′-dihydrospiro[cyclohexane-1,1′-indene]-4-carboxylic acid

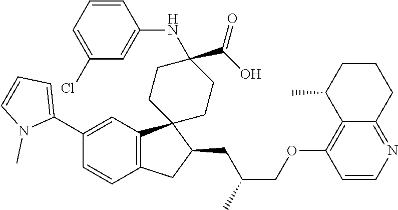 [SMILES: C[C@@H](COc1ccnc2c1[C@H](C)CCC2)C[C@H]1Cc2ccc(-c3cccn3C)cc2[C@]12CC[C@@](Nc1cccc(Cl)c1)(C(=O)O)CC2]

To an oven-dried microwave vial was added Preparation 16a (50 mg, 0.06 mmol, 1 eq), 1-methyl-2-(4,4,5,5-tetramethyl-1,3,2-dioxaborolan-2-yl)-1H-pyrrole (19 mg, 0.09 mmol, 1.5 eq), K₂CO₃ (25 mg, 0.18 mmol, 3 eq) in THE (2 mL) and water (0.2 mL). The mixture was sparged with N₂ before the addition of Pd(dppf)Cl₂×DCM (5 mg, 0.01 mmol, 0.1 eq) and the mixture was heated at 110° C. for 2 h under microwave irradiation. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-100% EtOAc in heptane afforded an intermediate, which was hydrolyzed as described in General procedure 33a to obtain Example 881 as a white powder. LRMS calculated for C₄₀H₄₆ClN₃O₃: 651; found: 652 (M+H). ¹H NMR (400 MHz, DMSO-d₆) δ ppm: 12.74 (br s, 1H), 8.16 (d, J=5.7 Hz, 1H), 7.46-7.41 (m, 1H), 7.28-7.20 (m, 2H), 7.05 (t, J=8.1 Hz, 1H), 6.85-6.82 (m, 1H), 6.79 (d, J=5.7 Hz, 1H), 6.62-6.49 (m, 3H), 6.30 (br s, 1H), 6.11-6.08 (m, 1H), 6.07-6.04 (m, 1H), 3.98-3.83 (m, 2H), 3.65 (s, 3H), 3.13-2.98 (m, 2H), 2.82-2.72 (m, 1H), 2.72-2.40 (m, 3H), 2.22-1.49 (m, 13H), 1.44-1.32 (m, 2H), 1.11-1.02 (m, 6H).

### Example 882 (1r,2′R,4R)-4-(3-chloroanilino)-2′-[(2R)-2-methyl-3-{[(5R)-5-methyl-5,6,7,8-tetrahydroquinolin-4-yl]oxy}propyl]-6′-(1H-pyrrol-3-yl)-2′,3′-dihydrospiro[cyclohexane-1,1′-indene]-4-carboxylic acid

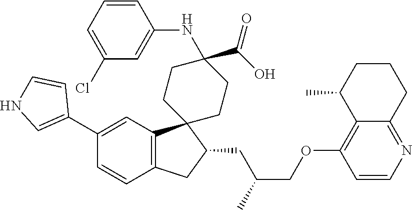 [SMILES: C[C@@H](COc1ccnc2c1[C@H](C)CCC2)C[C@@H]1Cc2ccc(-c3cc[nH]c3)cc2[C@]12CC[C@@](Nc1cccc(Cl)c1)(C(=O)O)CC2]

To an oven-dried microwave vial was added Preparation 16b (80 mg, 0.1 mmol, 1 eq), 3-(4,4,5,5-tetramethyl-1,3,2-dioxaborolan-2-yl)-1H-pyrrole (28 mg, 0.14 mmol, 1.5 eq), K₂CO₃ (40 mg, 0.29 mmol, 3 eq) in THF (2 mL) and water (0.2 mL). The mixture was sparged with N₂ before the addition of Pd(dppf)Cl₂×DCM (8 mg, 0.01 mmol, 0.1 eq) and the mixture was heated at 110° C. for 1.5 h under microwave irradiation. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-20% MeOH in DCM afforded an intermediate, which was hydrolyzed as described in General procedure 33a to obtain Example 882 as a white powder (5.5 mg, 8.62 μmol, 19%). LRMS calculated for C₃₉H₄₄ClN₃O₃: 637; found: 638 (M+H). ¹H NMR (400 MHz, DMSO-d₆) δ ppm: 10.91-10.85 (m, 1H), 8.14 (d, J=5.6 Hz, 1H), 7.58-7.55 (m, 1H), 7.30 (dd, J=7.7, 1.4 Hz, 1H), 7.15-7.02 (m, 3H), 6.81-6.77 (m, 2H), 6.62 (t, J=2.1 Hz, 1H), 6.58-6.49 (m, 2H), 6.37-6.33 (m, 1H), 4.03 (dd, J=9.7, 4.2 Hz, 1H), 3.89 (dd, J=9.7, 5.5 Hz, 1H), 3.09-2.93 (m, 2H), 2.80-2.70 (m, 1H), 2.67-2.49 (m, 3H), 2.23-1.97 (m, 4H), 1.93-1.61 (m, 6H), 1.61-1.32 (m, 4H), 1.32-1.21 (m, 1H), 1.15-1.08 (m, 6H).

### Example 883 (1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-(1-methyl-1H-pyrrol-3-yl)-2′,3′-dihydrospiro[cyclohexane-1,1′-indene]-4-carboxylic acid

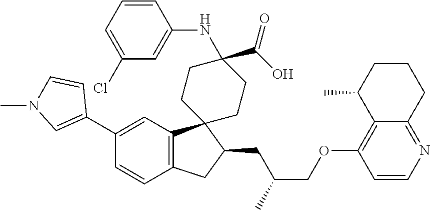 [SMILES: C[C@@H](COc1ccnc2c1[C@H](C)CCC2)C[C@H]1Cc2ccc(-c3ccn(C)c3)cc2[C@]12CC[C@@](Nc1cccc(Cl)c1)(C(=O)O)CC2]

To an oven-dried microwave vial was added Preparation 16a (50 mg, 0.06 mmol, 1 eq), 1-methyl-3-(4,4,5,5-tetramethyl-1,3,2-dioxaborolan-2-yl)-1H-pyrrole (19 mg, 0.09 mmol, 1.5 eq), K₂CO₃ (25 mg, 0.18 mmol, 3 eq) in THE (2 mL) and water (0.2 mL). The mixture was sparged with N₂ before the addition of Pd(dppf)Cl₂×DCM (5 mg, 0.01 mmol, 0.1 eq) and the mixture was heated at 110° C. for 1 h under microwave irradiation. The reaction was partitioned between DCM and water, and the organic phase was washed with brine, dried (MgSO₄) and concentrated in vacuo. Purification by automated flash chromatography (CombiFlash Rf, 12 g RediSep™ silica cartridge) eluting with a gradient of 0-100% EtOAc in heptane afforded an intermediate, which was hydrolyzed as described in General procedure 33a to obtain Example 883 as a white powder (2.6 mg, 3.99 μmol, 7%). LRMS calculated for C₄₀H₄₆ClN₃O₃: 651; found: 652 (M+H). ¹H NMR (400 MHz, DMSO-d₆) δ ppm: 8.15 (d, J=5.6 Hz, 1H), 7.50-7.47 (m, 1H), 7.26 (dd, J=7.7, 1.5 Hz, 1H), 7.11 (d, J=7.7 Hz, 1H), 7.07-7.00 (m, 2H), 6.77 (d, J=5.6 Hz, 1H), 6.74-6.72 (m, 1H), 6.64 (t, J=2.1 Hz, 1H), 6.58-6.49 (m, 2H), 6.31 (dd, J=2.7, 1.8 Hz, 1H), 3.95-3.83 (m, 2H), 3.64 (s, 3H), 3.11-3.02 (m, 1H), 2.97 (dd, J=15.6, 7.1 Hz, 1H), 2.81-2.72 (m, 1H), 2.71-2.43 (m, 3H), 2.26-2.12 (m, 2H), 2.07-1.93 (m, 2H), 1.91-1.31 (m, 11H), 1.09-1.02 (m, 6H).

### Example 885 (1r,2′S,4S)-4-(3-chloroanilino)-6′-[1-(4-hydroxyphenyl)ethyl]-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

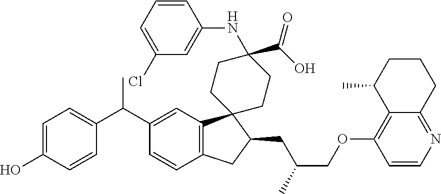 [SMILES: CC(c1ccc(O)cc1)c1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and a mixture of Example 859A and Example 860A as the appropriate indane and phenol as the appropriate alcohol, Example 885 was obtained unexpectedly as a mixture of diastereoisomers. ¹H NMR (500 MHz, DMSO-d₆) δ ppm: 8.13 (d, 1H), 7.71/7.62 (br s/br s, 1H), 7.07/7.06 (dd/dd, 1H), 7.06/6.95 (m/m, 2H), 7.02/7.00 (d/d, 1H), 6.94 (t, 1H), 6.78/6.69 (m/m, 2H), 6.75 (d, 1H), 6.63/6.60 (t/t, 1H), 6.54/6.52 (dm/dm, 1H), 6.41/6.39 (br d/br d, 1H), 4.48/4.37 (q/q, 1H), 3.89/3.82 (m+m, 2H), 3.03 (m, 1H), 2.89/2.42 (dd+dd, 2H), 2.75/2.64 (dm+m, 2H), 2.50-1.27 (m, 16H), 1.53/1.52 (d/d, 3H), 1.03 (d, 3H), 1.02 (d, 3H). HRMS calculated for C₄₃H₄₉N₂O₄Cl: 692.3381; found: 693.3447 and 693.3433 (M+H).

### Example 886

### Example 886A (5R)-3-chloro-5-methyl-5,6,7,8-tetrahydroquinolin-4-ol

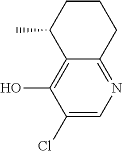

Preparation 2a1 (300 mg, 1.84 mmol) was dissolved in DMF (4.6 mL), then NCS (295 mg, 2.21 mmol) was added portionwise to the stirred suspension. The reaction mixture was stirred at rt until no further conversion was observed. The reaction mixture was diluted with brine, and extracted with THF. The combined organic layers were dried over Na₂SO₄, filtered and the filtrate was concentrated under reduced pressure. The crude product was purified via flash chromatography using DCM and MeOH as eluents to obtain Example 886A. ¹H NMR (500 MHz, DMSO-d₆) δ ppm: 11.53 (br s, 1H), 7.92 (d, 1H), 2.90 (m, 1H), 2.51 (m, 2H), 1.70 (m, 2H), 1.57 (m, 2H), 1.06 (d, 3H). HRMS calculated for C₁₀H₁₂ClNO: 197.0607; found 198.0675 (M+H).

### Example 886 (1r,4S,6′S)-4-(3-chloro-4-fluoroanilino)-6′-[(2R)-3-{[(5R)-3-chloro-5-methyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid

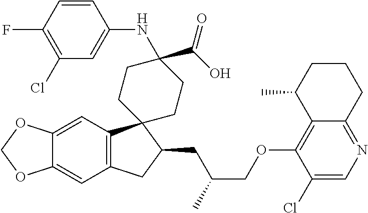 [SMILES: C[C@@H](COc1c(Cl)cnc2c1[C@H](C)CCC2)C[C@H]1Cc2cc3c(cc2[C@]12CC[C@@](Nc1ccc(F)c(Cl)c1)(C(=O)O)CC2)OCO3]

Using General procedure 32 and Example 845E as the appropriate indane and Example 886A as the appropriate alcohol, Example 886 was obtained. HRMS calculated for C₃₆H₃₉N₂O₅FCl₂: 668.2220; found: 669.2293 (M+H).

### Example 887 (1r,4S,6′S)-4-(3,4-dichloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

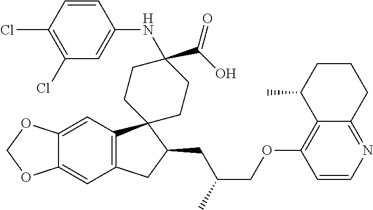 [SMILES: C[C@@H](COc1ccnc2c1[C@H](C)CCC2)C[C@H]1Cc2cc3c(cc2[C@]12CC[C@@](Nc1ccc(Cl)c(Cl)c1)(C(=O)O)CC2)OCO3]

A 4 mL-vial was charged with Example 839 (0.1 g, 0.158 mmol), NCS (0.021 g, 0.158 mmol) and MeCN (1.6 mL). The mixture was stirred for 12 h at rt, then NCS (0.01 g, 0.075 mmol) was added and the mixture was stirred for 12 h at rt. Then the mixture was purified via prep RP-HPLC using 25 mM aq. NH₄HCO₃ solution, IPA and MeCN as eluents to obtain an intermediate, which was hydrolyzed according to General Procedure 33a to obtain Example 887. ¹H NMR (500 MHz, DMSO-d₆) δ ppm: 12.73 (br s, 1H), 8.14 (d, 1H), 7.23 (d, 1H), 6.85 (s, 1H), 6.77 (d, 1H), 6.76 (d, 1H), 6.76 (s, 1H), 6.55 (dd, 1H), 6.36 (br s, 1H), 5.95/5.93 (d+d, 2H), 3.89/3.85 (dd+dd, 2H), 3.04 (m, 1H), 2.90/2.43 (dd+dd, 2H), 2.76/2.65 (m+m, 2H), 2.43-1.32 (m, 8H), 2.14 (m, 1H), 1.97 (m, 1H), 1.80/1.74 (m+m, 2H), 1.67/1.61 (m+m, 2H), 1.48/1.29 (t+t, 2H), 1.04 (d, 3H), 1.03 (d, 3H). HRMS calculated for C₃₆H₄₀Cl₂N₂O₅: 650.2314; found 651.2390 (M+H).

### Example 888

### Example 888A methyl (1r,4S,6′S)-4-[(3-chlorophenyl)(trifluoroacetyl)amino]-6′-{(2R)-2-methyl-3-[(trifluoroacetyl)oxy]propyl}-6′,7′-dihydro-2′H-spiro[cyclohexane-1,5′-indeno[5,6-d][1,3]dioxole]-4-carboxylate

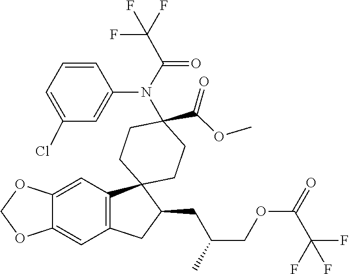 [SMILES: COC(=O)[C@]1(N(C(=O)C(F)(F)F)c2cccc(Cl)c2)CC[C@@]2(CC1)c1cc3c(cc1C[C@@H]2C[C@@H](C)COC(=O)C(F)(F)F)OCO3]

Example 839E (1.50 g, 3.09 mmol, 1 eq) was dissolved in 2-Me-THF (15 mL), then TEA (2.15 mL, 15.4 mmol, 5 eq.) and DMAP (37.7 mg, 0.309 mmol, 0.1 eq.) were added, then cooled to 0° C. TFAA (4.29 mL, 30.9 mmol, 10 eq.) was added dropwise at 0° C. (keeping the temperature of the mixture below 10° C.), then it was stirred at rt until no further conversion was observed. The organic layer was washed with sat. aq. NaHCO₃ solution, dried over Na₂SO₄, filtered and the filtrate was concentrated under reduced pressure. The crude product was purified via flash chromatography using heptane and EtOAc as eluents to obtain Example 888A (1.86 g, 2.75 mmol, 89%). ¹H NMR (500 MHz, DMSO-d₆) δ ppm: 7.78-7.41 (m, 4H), 6.68 (s, 1H), 6.58 (s, 1H), 5.93/5.91 (s/s, 2H), 4.17-4.07 (m, 2H), 3.79 (s, 3H), 2.89/2.34 (m+d, 2H), 2.44-0.75 (m, 10H), 2.22/2.17 (m/m, 1H), 1.83 (m, 1H), 0.84/0.82 (d/d, 3H). LRMS calculated for C₃₁H₃₀ClF₆NO₇: 677.1614; found: 676.1 (M−H).

### Example 888B methyl (1r,4S,6′S)-8′-chloro-4-[(3-chlorophenyl)(trifluoroacetyl)amino]-6′-{(2R)-2-methyl-3-[(trifluoroacetyl)oxy]propyl}-6′,7′-dihydro-2′H-spiro[cyclohexane-1,5′-indeno[5,6-d][1,3]dioxole]-4-carboxylate

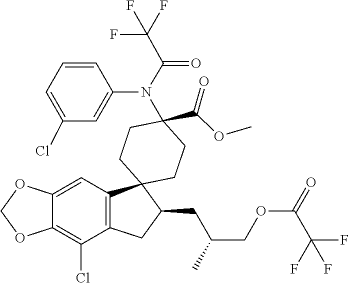 [SMILES: COC(=O)[C@]1(N(C(=O)C(F)(F)F)c2cccc(Cl)c2)CC[C@@]2(CC1)c1cc3c(c(Cl)c1C[C@@H]2C[C@@H](C)COC(=O)C(F)(F)F)OCO3]

To a solution of Example 888A (1.00 g, 1.48 mmol, 1 eq) in MeCN (15 mL) was added 1,3-dichloro-5,5-dimethyl-imidazolidine-2,4-dione (291 mg, 1.48 mmol, 1 eq.), then it was stirred at rt until no further conversion was observed. Then it was quenched with sat. aq. Na₂S₂O₃ solution and the layers were separated. The organic layer was filtered, and the filtrate was purified via prep RP-HPLC using 25 mM aq. NH₄HCO₃ solution and MeCN as eluents to obtain Example 888B (240 mg, 0.337 mmol, 23%). ¹H NMR (500 MHz, DMSO-d₆) δ ppm: 7.75-7.44 (m, 4H), 6.61/6.60 (s/s, 1H), 6.06/6.05 (d/d, 2H), 4.19-4.08 (dd+dd, 2H), 3.79 (s, 3H), 2.91/2.89/2.41/2.40 (dd+d/dd+d, 2H), 2.43-1.16 (m, 8H), 2.27/2.22 (m/m, 1H), 1.85/1.82 (m/m, 1H), 1.00/0.91/0.91/0.82 (t+t/t+t, 2H), 0.85/0.83 (d/d, 3H). HRMS calculated for C₃₁H₂₉Cl₂F₆NO₇: 711.1225; found: 710.1132 (M−H).

### Example 888C methyl (1r,4S,6′S)-8′-chloro-4-[(3-chlorophenyl)(trifluoroacetyl)amino]-6′-[(2R)-3-hydroxy-2-methylpropyl]-6′,7′-dihydro-2′H-spiro[cyclohexane-1,5′-indeno[5,6-d][1,3]dioxole]-4-carboxylate

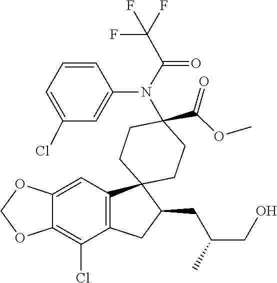 [SMILES: COC(=O)[C@]1(N(C(=O)C(F)(F)F)c2cccc(Cl)c2)CC[C@@]2(CC1)c1cc3c(c(Cl)c1C[C@@H]2C[C@@H](C)CO)OCO3]

To a solution of Example 888B (240 mg, 0.337 mmol, 1 eq) in MeOH (3.4 mL) was added K₂CO₃ (23.3 mg, 0.168 mmol, 0.5 eq.), then it was stirred at rt until no further conversion was observed. Then the mixture was concentrated under reduced pressure. The residue was dissolved in DCM and washed with brine. The organic layer was dried over Na₂SO₄, filtered and the filtrate was concentrated under reduced pressure to obtain Example 888C (208 mg, 0.337 mmol, 100%). ¹H NMR (500 MHz, DMSO-d₆) δ ppm: 7.73-7.42 (m, 4H), 6.61/6.60 (s, 1H), 6.05 (s, 2H), 4.40/4.37 (t, 1H), 3.79 (s, 3H), 3.19-2.97 (m, 2H), 2.88/2.42 (dd+dd, 2H), 2.46-1.16 (m, 8H), 2.25/2.20 (m, 1H), 1.44 (m, 1H), 1.08/0.98/0.71/0.60 (m+m, 2H), 0.73/0.70 (d, 3H). HRMS calculated for C₂₉H₃₀C₁₂F₃NO₆: 615.1402; found: 660.1370 (M+HCOO).

### Example 888 (1r,4S,6′S)-8′-chloro-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid

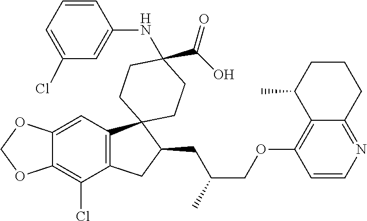 [SMILES: C[C@@H](COc1ccnc2c1[C@H](C)CCC2)C[C@H]1Cc2c(Cl)c3c(cc2[C@]12CC[C@@](Nc1cccc(Cl)c1)(C(=O)O)CC2)OCO3]

Using General procedure 32 and Example 888C as the appropriate indane and Preparation 2a1 as the appropriate aryl-alcohol, Example 888 was obtained. ¹H NMR (500 MHz, DMSO-d₆) δ ppm: 12.73 (br s, 1H), 8.14 (d, 1H), 7.03 (t, 1H), 6.83 (s, 1H), 6.76 (d, 1H), 6.61 (t, 1H), 6.53 (dm, 2H), 6.28 (br s, 1H), 6.08 (s, 2H), 3.87 (d, 2H), 3.04 (m, 1H), 2.96/2.46 (dd+dd, 2H), 2.76/2.65 (m+m, 2H), 2.42-1.37 (m, 12H), 2.22 (m, 1H), 1.98 (m, 1H), 1.49/1.30 (m+m, 2H), 1.04 (d, 3H), 1.00 (d, 3H). HRMS calculated for C₃₆H₄₀Cl₂N₂O₅: 650.2314; found: 651.2392 (M+H).

### Example 919 and Example 920

### Example 919A methyl (1r,4R)-4-(3-chloroanilino)-6′-(hydroxymethyl)-2′-{(2R)-3-[(4-methoxyphenyl)methoxy]-2-methylpropyl}spiro[cyclohexane-1,1′-indene]-4-carboxylate

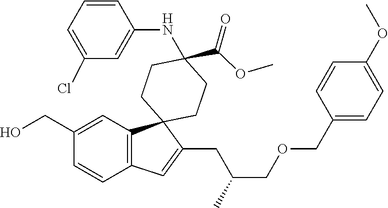 [SMILES: COc1ccc(COC[C@H](C)CC2=Cc3ccc(CO)cc3[C@]23CC[C@@](Nc2cccc(Cl)c2)(C(=O)OC)CC3)cc1]

Using General procedure 36 and Preparation 13aD as the appropriate aldehyde and THE instead of MeOH, Example 919A was obtained. LRMS calculated for C₃₅H₄₁ClNO₅: 589; found 590 (M+H).

### Example 919B methyl (1r,4R)-4-(3-chloroanilino)-6′-(methoxymethyl)-2′-{(2R)-3-[(4-methoxyphenyl)methoxy]-2-methylpropyl}spiro[cyclohexane-1,1′-indene]-4-carboxylate

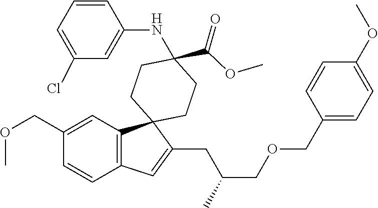 [SMILES: COCc1ccc2c(c1)[C@]1(CC[C@@](Nc3cccc(Cl)c3)(C(=O)OC)CC1)C(C[C@@H](C)COCc1ccc(OC)cc1)=C2]

To a solution of Example 919A (2.18 g, 3.69 mmol, 1 eq) in CHCl₃ (100 mL) was added Ag₂O (3.42 g, 14.78 mmol, 4 eq) and Mel (1.84 mL, 2.28 g/mL, 29.55 mmol, 8 eq). The reaction was heated at 40° C. under an atmosphere of N₂ for 16 h. A further lot of Mel (6 eq) was added and the reaction was heated at 40° C. for 20 h. The reaction was diluted with DCM and filtered through celite and the filtrate was concentrated under reduced pressure. Purification by automated flash chromatography (CombiFlash Rf, 80 g RediSep™ silica cartridge) eluting with a gradient of 0-30% EtOAc in heptane afforded Example 919B as an off-white gum (2.07 g, 3.43 mmol, 93%). LRMS calculated for C₃₆H₄₂ClNO₅: 603; found 604 (M+H). ¹H NMR (400 MHz, CDCl₃-d) δ ppm: 7.72-7.65 (m, 1H), 7.29-7.19 (m, 4H), 7.05 (t, J=8.0 Hz, 1H), 6.90-6.82 (m, 2H), 6.75-6.69 (m, 1H), 6.62 (t, J=2.2 Hz, 1H), 6.50-6.44 (m, 1H), 6.40 (s, 1H), 4.49 (s, 2H), 4.48-4.39 (m, 2H), 4.18 (s, 1H), 3.76 (s, 3H), 3.75 (s, 3H), 3.42 (s, 3H), 3.41-3.31 (m, 2H), 2.67-2.53 (m, 2H), 2.49-2.39 (m, 1H), 2.32-2.13 (m, 3H), 2.13-1.93 (m, 3H), 1.13-0.98 (m, 5H).

### Example 919C methyl (1r,4R)-4-(3-chloroanilino)-6′-(methoxymethyl)-2′-{(2R)-3-[(4-methoxyphenyl)methoxy]-2-methylpropyl}-2′,3′-dihydrospiro[cyclohexane-1,1′-indene]-4-carboxylate

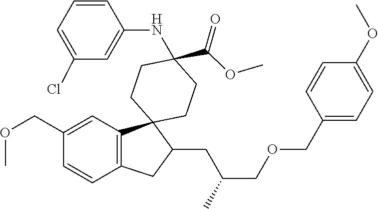

Using General procedure 19 with Example 919B as the appropriate indene, Example 919C was obtained as a mixture of diastereoisomers. LRMS calculated for C₃₆H₄₄ClNO₅: 605; found 606 (M+H).

### Example 919D methyl (1r,2′S,4S)-4-(3-chloroanilino)-2′-[(2R)-3-hydroxy-2-methylpropyl]-6′-(methoxymethyl)-2′,3′-dihydrospiro[cyclohexane-1,1′-indene]-4-carboxylate

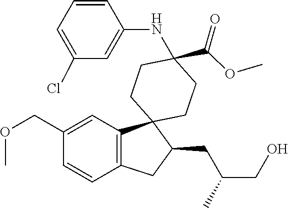 [SMILES: COCc1ccc2c(c1)[C@]1(CC[C@@](Nc3cccc(Cl)c3)(C(=O)OC)CC1)[C@@H](C[C@@H](C)CO)C2]

and

### Example 920A methyl (1r,2′R,4R)-4-(3-chloroanilino)-2′-[(2R)-3-hydroxy-2-methylpropyl]-6′-(methoxymethyl)-2′,3′-dihydrospiro[cyclohexane-1,1′-indene]-4-carboxylate

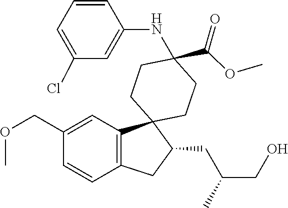 [SMILES: COCc1ccc2c(c1)[C@]1(CC[C@@](Nc3cccc(Cl)c3)(C(=O)OC)CC1)[C@H](C[C@@H](C)CO)C2]

Using General procedure 28a and Example 919C as the appropriate PMB derivative, a mixture of diastereoisomers was obtained. The diastereoisomers were separated via chiral chromatography. Column: AD, 100×500 mm, 20 μm, Eluents: iPrOH/heptane. The diastereoisomer eluting later was collected as Example 919D. ¹H NMR (500 MHz, DMSO-d₆) δ ppm: 7.28 (d, 1H), 7.16 (d, 1H), 7.08 (dd, 1H), 7.06 (t, 1H), 6.59 (t, 1H), 6.56 (dm, 1H), 6.45 (dm, 1H), 6.31 (s, 1H), 4.44 (t, 1H), 4.37 (s, 2H), 3.65 (s, 3H), 3.28 (s, 3H), 3.22/3.19 (m+m, 2H), 2.92/2.48 (dd+dd, 2H), 2.50-0.98 (m, 11H), 2.12 (m, 1H), 0.85 (d, 3H).

The diastereoisomer eluting earlier was collected as Example 920A. ¹H NMR (500 MHz, DMSO-d₆) δ ppm: 7.29 (d, 1H), 7.17 (d, 1H), 7.08 (dd, 1H), 7.06 (t, 1H), 6.59 (t, 1H), 6.56 (dm, 1H), 6.45 (dm, 1H), 6.29 (s, 1H), 4.39 (t, 1H), 4.37 (s, 2H), 3.65 (s, 3H), 3.42/3.20 (m+m, 2H), 3.28 (s, 3H), 2.95/2.52 (dd+dd, 2H), 2.50-0.93 (m, 11H), 2.11 (m, 1H), 0.90 (d, 3H).

### Example 919 (1r,2′S,4S)-4-(3-chloroanilino)-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

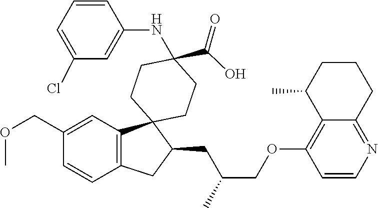 [SMILES: COCc1ccc2c(c1)[C@]1(CC[C@@](Nc3cccc(Cl)c3)(C(=O)O)CC1)[C@@H](C[C@@H](C)COc1ccnc3c1[C@H](C)CCC3)C2]

Using General procedure 32 and Example 919D as the appropriate indane and Preparation 2a1 as the appropriate alcohol, Example 919 was obtained. ¹H NMR (500 MHz, DMSO-d₆) δ ppm: 12.65 (br s, 1H), 8.14 (d, 1H), 7.28 (d, 1H), 7.17 (d, 1H), 7.08 (dd, 1H), 7.04 (t, 1H), 6.76 (d, 1H), 6.62 (t, 1H), 6.55 (dm, 1H), 6.54 (dm, 1H), 6.26 (br s, 1H), 4.38 (s, 2H), 3.89/3.84 (dd+dd, 2H), 3.27 (s, 3H), 3.04 (m, 1H), 2.99/2.52 (dd+dd, 2H), 2.75/2.65 (m+m, 2H), 2.50-1.36 (m, 12H), 2.18 (m, 1H), 2.00 (m, 1H), 1.46/1.33 (m+m, 2H), 1.05 (d, 3H), 1.03 (d, 3H). HRMS calculated for C₃₇H₄₅N₂O₄Cl: 616.3068; found: 617.3140 (M+H).

### Example 920 (1r,2′R,4R)-4-(3-chloroanilino)-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

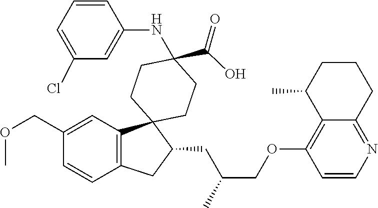 [SMILES: COCc1ccc2c(c1)[C@]1(CC[C@@](Nc3cccc(Cl)c3)(C(=O)O)CC1)[C@H](C[C@@H](C)COc1ccnc3c1[C@H](C)CCC3)C2]

Using General procedure 32 and Example 920A as the appropriate indane and Preparation 2a1 as the appropriate alcohol, Example 920 was obtained. ¹H NMR (500 MHz, DMSO-d₆) δ ppm: 12.7 (br s, 1H), 8.13 (d, 1H), 7.31 (s, 1H), 7.17 (d, 1H), 7.07 (dd, 1H), 7.03 (t, 1H), 6.78 (d, 1H), 6.61 (t, 1H), 6.54 (dd, 1H), 6.51 (dd, 1H), 6.12 (br s, 1H), 4.37 (s, 2H), 4.01/3.88 (dd+dd, 2H), 3.27 (s, 3H), 3.01 (m, 1H), 2.95/2.57 (dd+dd, 2H), 2.73/2.60 (m+m, 2H), 2.43-1.17 (m, 8H), 2.12 (m, 1H), 2.08 (m, 1H), 1.76/1.66 (m+m, 2H), 1.69/1.21 (m+m, 2H), 1.53/1.47 (m+m, 2H), 1.09 (d, 3H), 1.09 (d, 3H). HRMS calculated for C₃₇H₄₅N₂O₄Cl: 616.3068; found: 617.3120 (M+H).

### Example 921

### Example 921A methyl (1s,4s)-2′-bromo-4-[(3-chlorophenyl)(trifluoroacetyl)amino]-5′-fluorospiro[cyclohexane-1,1′-indene]-4-carboxylate

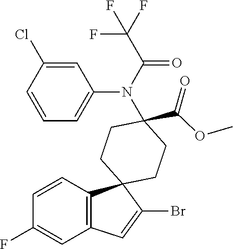 [SMILES: COC(=O)[C@]1(N(C(=O)C(F)(F)F)c2cccc(Cl)c2)CC[C@]2(CC1)C(Br)=Cc1cc(F)ccc12]

Using General procedure 22 and Example 737A as the appropriate indene, Example 921A was obtained as a yellow gum. ¹H NMR (400 MHz, DMSO-d₆) δ ppm: 7.84-7.80 (m, 1H), 7.72-7.61 (m, 3H), 7.48 (dd, J=8.4, 5.0 Hz, 1H), 7.19 (dd, J=8.9, 2.6 Hz, 1H), 7.11-7.04 (m, 1H), 7.03 (s, 1H), 3.84 (s, 3H), 2.49-2.24 (m, 4H), 1.83-1.64 (m, 1H), 1.60-1.36 (m, 3H).

### Example 921B methyl (1s,4s)-2′-bromo-4-[(3-chlorophenyl)(trifluoroacetyl)amino]-5′-fluoro-6′-formylspiro[cyclohexane-1,1′-indene]-4-carboxylate

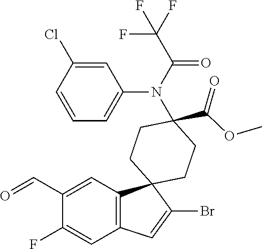 [SMILES: COC(=O)[C@]1(N(C(=O)C(F)(F)F)c2cccc(Cl)c2)CC[C@]2(CC1)C(Br)=Cc1cc(F)c(C=O)cc12]

Using General procedure 26 and Example 921A as the appropriate indene, Example 921B was obtained as a white foam. ¹H NMR (400 MHz, DMSO-d₆) δ ppm: 10.24 (s, 1H), 7.90 (d, J=6.1 Hz, 1H), 7.84-7.81 (m, 1H), 7.72-7.61 (m, 3H), 7.39 (d, J=10.5 Hz, 1H), 7.17 (s, 1H), 3.87 (s, 3H), 2.49-2.24 (m, 4H), 1.82-1.70 (m, 1H), 1.63-1.35 (m, 3H).

### Example 921C methyl (1r,4R)-4-[(3-chlorophenyl)(trifluoroacetyl)amino]-5′-fluoro-6′-formyl-2′-{(2R)-3-[(4-methoxyphenyl)methoxy]-2-methylpropyl}spiro[cyclohexane-1,1′-indene]-4-carboxylate

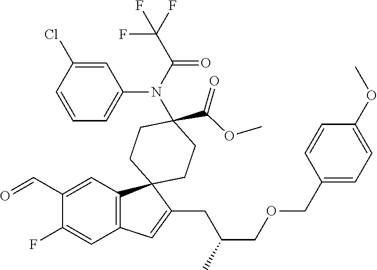 [SMILES: COc1ccc(COC[C@H](C)CC2=Cc3cc(F)c(C=O)cc3[C@]23CC[C@](C(=O)OC)(N(C(=O)C(F)(F)F)c2cccc(Cl)c2)CC3)cc1]

Using General procedure 27b and Example 921B as the appropriate 2-bromo-indene derivative and Preparation 3d as the appropriate Zn reagent, Example 921C was obtained as a yellow gum. ¹H NMR (400 MHz, DMSO-d₆) δ ppm: 10.22 (s, 1H), 7.96-7.80 (m, 2H), 7.75-7.62 (m, 2H), 7.60-7.50 (m, 1H), 7.30-7.21 (m, 3H), 6.93-6.87 (m, 2H), 6.61-6.55 (m, 1H), 4.47-4.34 (m, 2H), 3.87 (s, 3H), 3.74 (s, 3H), 3.40-3.27 (m, 2H), 2.63-2.24 (m, 4H), 2.22-2.10 (m, 2H), 2.06-1.87 (m, 1H), 1.75-1.63 (m, 1H), 1.47-1.34 (m, 1H), 1.24-1.01 (m, 2H), 0.98-0.89 (m, 3H).

### Example 921D (1r,4R)-4-(3-chloroanilino)-5′-fluoro-6′-formyl-2′-{(2R)-3-[(4-methoxyphenyl)methoxy]-2-methylpropyl}spiro[cyclohexane-1,1′-indene]-4-carboxylic acid

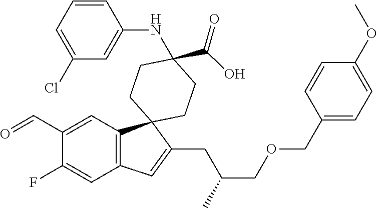 [SMILES: COc1ccc(COC[C@H](C)CC2=Cc3cc(F)c(C=O)cc3[C@]23CC[C@@](Nc2cccc(Cl)c2)(C(=O)O)CC3)cc1]

Using General procedure 33a and Example 921C as the appropriate ester, Example 921D was obtained. LRMS calculated for C₃₄H₃₅ClFNO₅: 591; found 592 (M+H). ¹H NMR (400 MHz, DMSO-d₆) δ ppm: 12.85 (br s, 1H), 10.24 (s, 1H), 8.03 (d, J=6.3 Hz, 1H), 7.30 (d, J=10.8 Hz, 1H), 7.25-7.19 (m, 2H), 7.10 (t, J=8.1 Hz, 1H), 6.89-6.83 (m, 2H), 6.66 (t, J=2.1 Hz, 1H), 6.63-6.54 (m, 3H), 6.35 (br s, 1H), 4.43-4.35 (m, 2H), 3.73 (s, 3H), 3.36-3.28 (m, 2H), 2.48-2.39 (m, 1H), 2.38-2.03 (m, 8H), 0.98-0.85 (m, 5H).

### Example 921E methyl (1r,4R)-4-(3-chloroanilino)-5′-fluoro-6′-formyl-2′-{(2R)-3-[(4-methoxyphenyl)methoxy]-2-methylpropyl}spiro[cyclohexane-1,1′-indene]-4-carboxylate

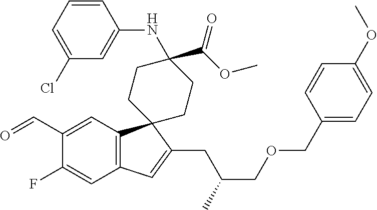 [SMILES: COc1ccc(COC[C@H](C)CC2=Cc3cc(F)c(C=O)cc3[C@]23CC[C@@](Nc2cccc(Cl)c2)(C(=O)OC)CC3)cc1]

Using General procedure 17a and Example 921D as the appropriate amino acid, Example 921E was obtained. LRMS calculated for C₃₅H₃₇ClFNO₅: 605; found 606 (M+H).

### Example 921F methyl (1r,4R)-4-(3-chloroanilino)-5′-fluoro-6′-(hydroxymethyl)-2′-{(2R)-3-[(4-methoxyphenyl)methoxy]-2-methylpropyl}spiro[cyclohexane-1,1′-indene]-4-carboxylate

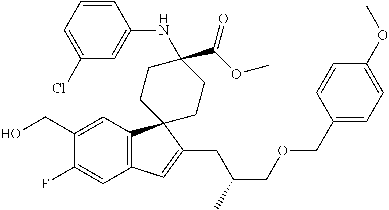 [SMILES: COc1ccc(COC[C@H](C)CC2=Cc3cc(F)c(CO)cc3[C@]23CC[C@@](Nc2cccc(Cl)c2)(C(=O)OC)CC3)cc1]

Using General procedure 36 and Example 921E as the appropriate aldehyde and THE instead of MeOH, Example 921F was obtained. LRMS calculated for C₃₅H₃₉ClFNO₅: 607; found 608 (M+H).

### Example 921G methyl (1r,4R)-4-(3-chloroanilino)-5′-fluoro-6′-(methoxymethyl)-2′-{(2R)-3-[(4-methoxyphenyl)methoxy]-2-methylpropyl}spiro[cyclohexane-1,1′-indene]-4-carboxylate

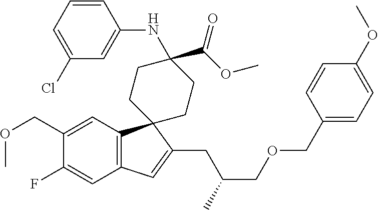 [SMILES: COCc1cc2c(cc1F)C=C(C[C@@H](C)COCc1ccc(OC)cc1)[C@]21CC[C@](Nc2cccc(Cl)c2)(C(=O)OC)CC1]

To a solution of Example 921F (521 mg, 0.86 mmol, 1 eq) in CHCl₃ (15 mL) was added Ag₂O (397.07 mg, 1.71 mmol, 2 eq) and Mel (213.43 μL, 2.28 g/mL, 3.43 mmol, 4 eq). The reaction was heated at 40° C. under an atmosphere of N₂ for 6 h. A further lot of Mel (8 eq) and Ag₂O (1 eq) were added and the reaction was heated at 40° C. for 24 h. The reaction was diluted with DCM and filtered through celite and then the filtrate was concentrated under reduced pressure. Purification by automated flash chromatography (CombiFlash Rf, 80 g RediSep™ silica cartridge) eluting with a gradient of 0-35% EtOAc in heptane afforded Example 921G as a colourless gum (475 mg, 0.76 mmol, 89%). LRMS calculated for C₃₆H₄₁ClFNO₅: 621; found 622 (M+H). ¹H NMR (400 MHz, DMSO-d₆) δ ppm: 7.66 (d, J=6.7 Hz, 1H), 7.25-7.19 (m, 2H), 7.13-7.06 (m, 2H), 6.89-6.83 (m, 2H), 6.65 (t, J=2.1 Hz, 1H), 6.63-6.58 (m, 1H), 6.51-6.46 (m, 1H), 6.46-6.43 (m, 1H), 6.41 (s, 1H), 4.47 (s, 2H), 4.43-4.34 (m, 2H), 3.73 (s, 3H), 3.70 (s, 3H), 3.37-3.25 (m, 5H), 2.43-2.30 (m, 3H), 2.27-1.95 (m, 6H), 0.94 (d, J=6.6 Hz, 3H), 0.91-0.83 (m, 2H).

### Example 921H methyl (1r,4R)-4-(3-chloroanilino)-5′-fluoro-6′-(methoxymethyl)-2′-{(2R)-3-[(4-methoxyphenyl)methoxy]-2-methylpropyl}-2′,3′-dihydrospiro[cyclohexane-1,1′-indene]-4-carboxylate

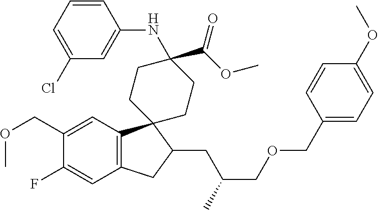

Using General procedure 19 with Example 921G as the appropriate indene, Example 921H was obtained as a mixture of diastereoisomers. LRMS calculated for C₃₆H₄₃ClFNO₅: 623; found 624 (M+H).

### Example 9211 methyl (1r,2′S,4S)-4-(3-chloroanilino)-2′-[(2R)-3-hydroxy-2-methylpropyl]-6′-(methoxymethyl)-2′,3′-dihydrospiro[cyclohexane-1,1′-indene]-4-carboxylate

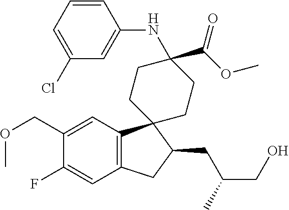 [SMILES: COCc1cc2c(cc1F)C[C@H](C[C@@H](C)CO)[C@]21CC[C@](Nc2cccc(Cl)c2)(C(=O)OC)CC1]

Using General procedure 28a and Example 921H as the appropriate PMB derivative, a mixture of diastereoisomers was obtained. The diastereoisomers were separated via chiral chromatography. Column: AD, 50×500 mm, 20 μm, Eluents: iPrOH/heptane. The diastereoisomer eluting later was collected as Example 9211. ¹H NMR (500 MHz, DMSO-d₆) δ ppm: 7.36 (d, 1H), 7.06 (t, 1H), 7.03 (d, 1H), 6.59 (t, 1H), 6.57 (dm, 1H), 6.45 (dm, 1H), 6.29 (s, 1H), 4.42 (s, 2H), 4.40 (t, 1H), 3.64 (s, 3H), 3.40/3.20 (m+m, 2H), 3.30 (s, 3H), 2.97/2.54 (dd+dd, 2H), 2.46-0.94 (m, 10H), 2.13 (m, 1H), 1.59 (m, 1H), 0.90 (d, 3H). HRMS calculated for C₂₈H₃₅NO₄ClF: 503.2239; found: 504.2333 (M+H).

### Example 921 (1r,2′S,4S)-4-(3-chloroanilino)-5′-fluoro-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid

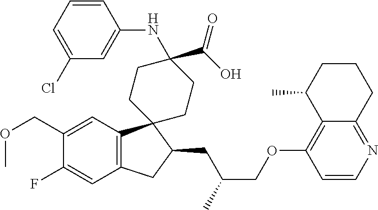 [SMILES: COCc1cc2c(cc1F)C[C@H](C[C@@H](C)COc1ccnc3c1[C@H](C)CCC3)[C@]21CC[C@](Nc2cccc(Cl)c2)(C(=O)O)CC1]

Using General procedure 32 and Example 9211 as the appropriate indane and Preparation 2a1 as the appropriate alcohol, Example 921 was obtained. ¹H NMR (500 MHz, DMSO-d₆) δ ppm: 12.61 (br s, 1H), 8.14 (d, 1H), 7.33 (d, 1H), 7.04 (t, 1H), 7.03 (d, 1H), 6.77 (d, 1H), 6.62 (t, 1H), 6.54 (dm, 2H), 6.26 (br s, 1H), 4.42 (s, 2H), 3.89/3.85 (dd+dd, 2H), 3.29 (s, 3H), 3.03 (m, 1H), 3.01/2.54 (dd+dd, 2H), 2.76/2.65 (m+m, 2H), 2.48-1.35 (m, 12H), 2.20 (m, 1H), 1.98 (m, 1H), 1.45/1.32 (m+m, 2H), 1.04 (d, 3H), 1.02 (d, 3H). HRMS calculated for C₃₇H₄₄N₂O₄FCl: 634.2974; found: 635.3052 (M+H).

### Example 930 ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

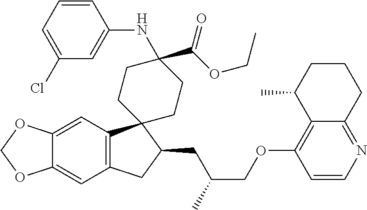 [SMILES: CCOC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Example 840 (100 mg, 0.162 mmol) was dissolved in DMF (1.62 mL), then CsF (49.2 mg, 0.324 mmol) and EtI (314 mg, 0.162 mL, 0.324 mmol) were added. The reaction mixture was stirred at 35° C. until no further conversion was observed. The reaction mixture was neutralized with 2 M aq. HCl. The product was isolated and purified by prep RP-HPLC using 25 mM aq. NH₄HCO₃ solution and MeCN as eluents resulting Example 930. ¹H NMR (500 MHz, DMSO-d₆) δ ppm: 8.14 (d, 1H), 7.04 (t, 1H), 6.87 (s, 1H), 6.77 (s, 1H), 6.76 (d, 1H), 6.58 (t, 1H), 6.55 (dd, 1H), 6.47 (dd, 1H), 6.28 (s, 1H), 5.95/5.94 (d+d, 2H), 4.14 (q, 2H), 3.89/3.84 (dd+dd, 2H), 3.04 (m, 1H), 2.90/2.43 (dd+dd, 2H), 2.76/2.65 (m+m, 2H), 2.44-1.31 (m, 8H), 2.15 (m, 1H), 1.97 (m, 1H), 1.80/1.74 (m+m, 2H), 1.68/1.60 (m+m, 2H), 1.47/1.30 (t+t, 2H), 1.11 (t, 3H), 1.05 (d, 3H), 1.03 (d, 3H). HRMS calculated for C₃₈H₄₅N₂O₅Cl: 644.3017; found: 645.3091 (M+H).

### Example 931 propan-2-yl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

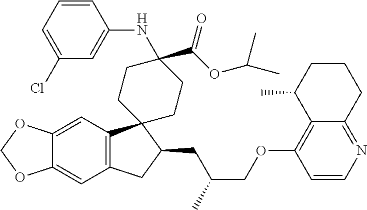 [SMILES: CC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Example 840 (100 mg, 0.162 mmol) and PPh₃ (85.0 mg, 0.324 mmol) were dissolved in THE (1.62 mL). The solution was heated to 50° C., then iPrOH (0.025 mL, 0.324 mmol) and DTBAD (74.6 mg, 0.324 mmol) were added. The reaction mixture was stirred at 50° C. under N₂ until no further conversion was observed. The product was isolated and purified by prep RP-HPLC using 25 mM aq. NH₄HCO₃ solution and MeCN as eluents resulting Example 931. ¹H NMR (500 MHz, DMSO-d₆) δ ppm: 8.14 (d, 1H), 7.04 (t, 1H), 6.84 (s, 1H), 6.77 (s, 1H), 6.76 (d, 1H), 6.57 (t, 1H), 6.55 (dm, 1H), 6.49 (dm, 1H), 6.25 (s, 1H), 5.95 (s, 2H), 4.97 (sept., 1H), 3.89/3.84 (dd+dd, 2H), 3.65/2.76 (dm+m, 2H), 3.04 (m, 1H), 2.90/2.43 (dd+dd, 2H), 2.44-1.25 (m, 14H), 2.16 (m, 1H), 1.96 (m, 1H), 1.14/1.12 (d+d, 6H), 1.05 (d, 3H), 1.03 (d, 3H). HRMS calculated for C₃₉H₄₇N₂O₅Cl: 658.3173; found: 659.3253 (M+H).

### Example 932 2,2,2-trifluoroethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

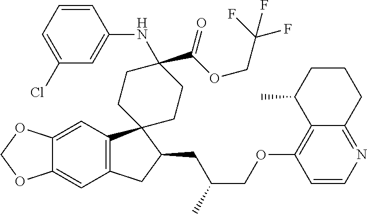 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCC(F)(F)F)CC2)OCO3]

Example 840 (100 mg, 0.162 mmol) was dissolved in THE (1.62 mL), then TEA (0.0452 mL, 0.324 mmol) and HBTU (122.9 mg, 0.324 mmol) were added. To the stirred reaction mixture 2,2,2-trifluoroethanol (0.024 mL, 0.324 mmol) was added at rt. The reaction mixture was stirred at rt until no further conversion was observed. The reaction mixture was diluted with water and brine and extracted with EtOAc. The combined organic layers were dried over Na₂SO₄, filtered and the filtrate was concentrated under reduced pressure. The crude product was purified by prep RP-HPLC using 25 mM aq. NH₄HCO₃ solution and MeCN as eluents resulting Example 932. ¹H NMR (500 MHz, DMSO-d₆) δ ppm: 8.14 (d, 1H), 7.04 (t, 1H), 6.95 (m, 2H), 6.86 (s, 1H), 6.77 (s, 1H), 6.76 (d, 1H), 6.60 (t, 1H), 6.58 (dm, 1H), 6.48 (dm, 1H), 6.39 (s, 1H), 4.86 (q, 2H), 3.89/3.85 (dd+dd, 2H), 3.04 (m, 1H), 2.90/2.43 (dd+dd, 2H), 2.76/2.65 (dm+m, 2H), 2.48-1.25 (m, 14H), 2.16 (m, 1H), 1.97 (m, 1H), 1.05 (d, 3H), 1.03 (d, 3H). HRMS calculated for C₃₈H₄₂N₂O₅F₃Cl: 698.2734; found: 699.2808 (M+H).

### Example 933 2-(2-methoxyethoxy)ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

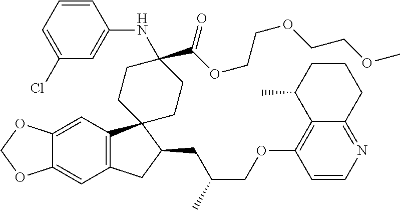 [SMILES: COCCOCCOC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Example 840 (100 mg, 0.162 mmol) and PPh₃ (85.0 mg, 0.324 mmol) were dissolved in THE (1.62 mL). The solution was heated to 50° C., then 2-(2-methoxyethoxy)ethanol in THE (2 M, 0.162 mL, 0.324 mmol) and DTBAD (74.6 mg, 0.324 mmol) were added. The reaction mixture was stirred at 50° C. under N₂ until no further conversion was observed. The product was isolated and purified by prep RP-HPLC using 25 mM aq. NH₄HCO₃ solution and MeCN as eluents resulting Example 933. ¹H NMR (500 MHz, DMSO-d₆) δ ppm: 8.14 (d, 1H), 7.03 (t, 1H), 6.88 (s, 1H), 6.76 (s, 1H), 6.76 (d, 1H), 6.59 (t, 1H), 6.55 (dm, 1H), 6.47 (dm, 1H), 6.31 (s, 1H), 5.95/5.94 (d+d, 2H), 4.23 (m, 2H), 3.89/3.84 (dd+dd, 2H), 3.55 (m, 2H), 3.44 (m, 2H), 3.34 (m, 2H), 3.18 (s, 3H), 3.04 (m, 1H), 2.90/2.43 (dd+dd, 2H), 2.76/2.65 (m+m, 2H), 2.48-1.31 (m, 12H), 2.15 (m, 1H), 1.97 (m, 1H), 1.47/1.30 (m+m, 2H), 1.04 (d, 3H), 1.04 (d, 3H). HRMS calculated for C₄₁H₅₁N₂O₇Cl: 718.3385; found: 719.3461 (M+H).

### Example 934 2-methoxyethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

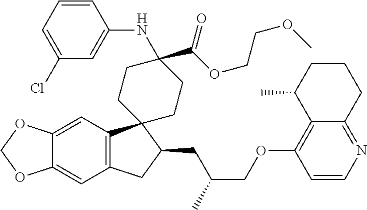 [SMILES: COCCOC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Example 840 (100 mg, 0.162 mmol), PPh₃ (85.0 mg, 0.324 mmol) and 2-methoxyethanol (0.026 mL, 0.324 mmol) were dissolved in THE (1.62 mL), then DTBAD (74.6 mg, 0.324 mmol) was added. The reaction mixture was stirred at 50° C. under N₂ until no further conversion was observed. The product was isolated and purified by prep RP-HPLC using 25 mM aq. NH₄HCO₃ solution and MeCN as eluents resulting Example 934. HRMS calculated for C₃₉H₄₇N₂O₆Cl: 674.3123; found: 675.3199 (M+H).

### Example 935 2-(dimethylamino)ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

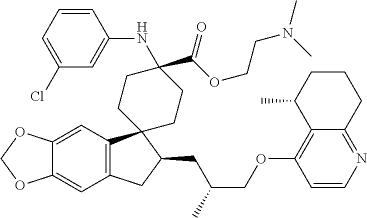 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCCN(C)C)CC2)OCO3]

Example 840 (100 mg, 0.162 mmol) was dissolved in THE (1.62 mL), then TEA (0.0452 mL, 0.324 mmol) and HBTU (122.9 mg, 0.324 mmol) were added. To the stirred reaction mixture 2-(dimethylamino)ethanol (0.033 mL, 0.324 mmol) was added at rt. The reaction mixture was stirred at rt until no further conversion was observed. The crude product was purified by prep RP-HPLC using 25 mM aq. NH₄HCO₃ solution and MeCN as eluents resulting Example 935. ¹H NMR (500 MHz, DMSO-d₆) δ ppm: 8.14 (d, 1H), 7.03 (t, 1H), 6.89 (s, 1H), 6.76 (s, 1H), 6.76 (d, 1H), 6.58 (t, 1H), 6.55 (dm, 1H), 6.49 (dm, 1H), 6.29 (s, 1H), 5.95/5.94 (d+d, 2H), 4.20/4.17 (m+m, 2H), 3.89/3.84 (dd+dd, 2H), 3.04 (m, 1H), 2.90/2.43 (dd+dd, 2H), 2.76/2.66 (m+m, 2H), 2.50-1.32 (m, 12H), 2.42 (t, 2H), 2.15 (m, 1H), 2.11 (s, 6H), 1.97 (m, 1H), 1.46/1.3 (m+m, 2H), 1.04 (d, 3H), 1.03 (d, 3H). HRMS calculated for C₄₀H₅₀N₃O₅Cl: 687.3439; found: 688.3510 (M+H).

### Example 936 2-(dimethylamino)-2-oxoethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

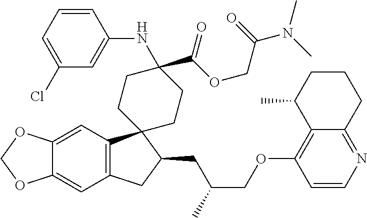 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCC(=O)N(C)C)CC2)OCO3]

Example 840 (86.4 mg, 0.140 mmol), NaI (31.5 mg, 0.210 mmol), 2-chloro-N,N-dimethyl-acetamide (0.022 mL, 0.210 mmol) were suspended in DMF (1.4 mL). To the stirred mixture Cs₂CO₃ (68.4 mg, 0.210 mmol) was added. The reaction mixture was stirred at rt until no further conversion was observed. The reaction mixture was diluted with water and brine and was extracted with EtOAc. The combined organic layers were dried over Na₂SO₄, filtered and the filtrate was concentrated under reduced pressure. The crude product was purified by prep RP-HPLC using 25 mM aq. NH₄HCO₃ solution and MeCN as eluents resulting Example 936. ¹H NMR (500 MHz, DMSO-d₆) δ ppm: 8.14 (d, 1H), 7.03 (t, 1H), 6.95 (s, 1H), 6.77 (d, 1H), 6.76 (s, 1H), 6.65 (t, 1H), 6.55 (dm, 1H), 6.53 (dm, 1H), 6.30 (s, 1H), 5.94/5.93 (d+d, 2H), 4.88/4.86 (d+d, 2H), 3.90/3.85 (dd+dd, 2H), 3.05 (m, 1H), 2.91/2.43 (dd+dd, 2H), 2.91 (s, 3H), 2.84 (s, 3H), 2.76/2.65 (m+m, 2H), 2.63-1.45 (m, 12H), 2.17 (m, 1H), 1.97 (m, 1H), 1.48/1.31 (m+m, 2H), 1.05 (d, 3H), 1.04 (d, 3H). HRMS calculated for C₄₀H₄₈N₃O₆Cl: 701.3232; found: 702.3311 (M+H).

### Example 937 2,3-dihydro-1H-inden-5-yl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

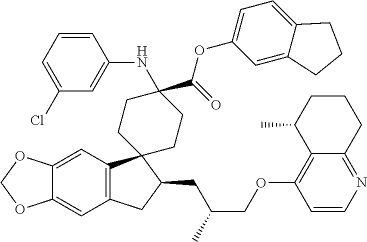 [SMILES: C[C@@H](COc1ccnc2c1[C@H](C)CCC2)C[C@H]1Cc2cc3c(cc2[C@]12CC[C@@](Nc1cccc(Cl)c1)(C(=O)Oc1ccc4c(c1)CCC4)CC2)OCO3]

Example 840 (100 mg, 0.162 mmol) was dissolved in dry THE (3.24 mL), then indan-5-ol (43.5 mg, 0.324 mmol), HBTU (122.9 mg, 0.324 mmol) and TEA (0.045 mL, 0.324 mmol) were added. The reaction mixture was stirred at 40° C. under N₂ atmosphere for 3 hours. To the reaction mixture indan-5-ol (43.5 mg, 0.324 mmol), HBTU (122.9 mg, 0.324 mmol) and TEA (0.045 mL, 0.324 mmol) were added. The reaction mixture was stirred at 40° C. under N₂ atmosphere until no further conversion was observed. The reaction mixture was diluted with water and brine and extracted with EtOAc. The combined organic layers were washed with brine, dried over Na₂SO₄, filtered and the filtrate was concentrated under reduced pressure. The crude product was purified by prep RP-HPLC using 25 mM aq. NH₄HCO₃ solution and MeCN as eluents resulting Example 937. ¹H NMR (500 MHz, DMSO-d₆) δ ppm: 8.15 (d, 1H), 7.21 (d, 1H), 7.13 (t, 1H), 6.94 (s, 1H), 6.79 (d, 1H), 6.78 (d, 1H), 6.78 (s, 1H), 6.71 (t, 1H), 6.63 (dm, 1H), 6.62 (dd, 1H), 6.60 (dm, 1H), 6.50 (s, 1H), 5.95/5.94 (d+d, 2H), 3.91/3.86 (dd+dd, 2H), 3.06 (m, 1H), 2.92/2.45 (dd+dd, 2H), 2.83 (t, 2H), 2.82 (t, 2H), 2.77/2.65 (m+m, 2H), 2.64-1.34 (m, 12H), 2.18 (m, 1H), 2.02 (m, 2H), 1.99 (m, 1H), 1.52/1.34 (m+m, 2H), 1.06 (d, 3H), 1.06 (d, 3H). HRMS calculated for C₄₅H₄₉N₂O₅Cl: 732.3330; found: 733.3407 (M+H).

### Example 938 (5-methyl-2-oxo-2H-1,3-dioxol-4-yl)methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

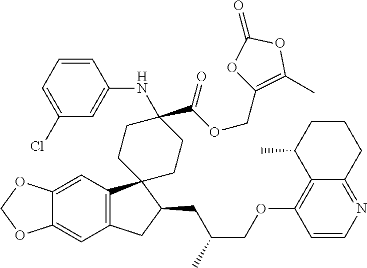 [SMILES: Cc1oc(=O)oc1COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Example 840 (100 mg, 0.162 mmol) was dissolved dry DMF (1.62 mL), then NaI (48.6 mg, 0.324 mmol) and Cs₂CO₃ (105.6 mg, 0.324 mmol) were added. 4-(Chloromethyl)-5-methyl-1,3-dioxol-2-one (48.1 mg, 0.324 mmol) was dissolved in dry DMF (0.690 mL), and the solution was added to the reaction mixture. Then it was stirred at rt until no further conversion was observed. The reaction mixture was diluted with water and brine, and was extracted with EtOAc. The combined organic layers were washed with brine, dried over Na₂SO₄, filtered and the filtrate was concentrated under reduced pressure. The crude product was purified by prep RP-HPLC using 25 mM aq. NH₄HCO₃ solution and MeCN as eluents resulting Example 938. ¹H NMR (500 MHz, DMSO-d₆) δ ppm: 8.14 (d, 1H), 6.99 (t, 1H), 6.84 (s, 1H), 6.77 (d, 1H), 6.76 (s, 1H), 6.54 (dd, 1H), 6.53 (t, 1H), 6.45 (dd, 1H), 6.32 (s, 1H), 5.95/5.94 (d+d, 2H), 5.06/5.03 (d+d, 2H), 3.89/3.84 (dd+dd, 2H), 3.03 (m, 1H), 2.90/2.43 (dd+dd, 2H), 2.76/2.65 (m+m, 2H), 2.46-1.34 (m, 8H), 2.17 (m, 1H), 2.12 (s, 3H), 1.97 (m, 1H), 1.80/1.73 (m+m, 2H), 1.67/1.60 (m+m, 2H), 1.46/1.29 (t+t, 2H), 1.04 (d, 3H), 1.03 (d, 3H). HRMS calculated for C₄₁H₄₅N₂O₈Cl: 728.2864; found: 729.2944 (M+H).

### Example 939 1-(acetyloxy)ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

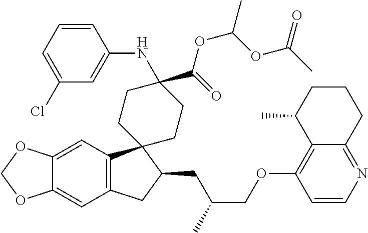 [SMILES: CC(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

2,4,6-Trimethyl-1,3,5-trioxane (1.44 mL, 10.8 mml) and NaI (5130 mg, 34.2 mmol) was dissolved in DCM (100 mL), then AcCl (2.03 mL, 28.5 mmol) was added. The reaction mixture was stirred at rt overnight. The reaction mixture was filtered, and the filtrate was concentrated under reduced pressure resulting 1-iodoethyl acetate as a racemate. Example 840 (100 mg, 0.162 mmol) and Cs₂CO₃ (316.7 mg, 0.9721 mmol) were suspended in dry DMF (3.24 mL), then 1-iodoethyl acetate (173.4 mg, 0.8101 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 0.1 V/V % aq. TFA solution and MeCN as eluents resulting Example 939 as a mixture of diastereoisomers and as TFA salt. HRMS calculated for C₄₀H₄₇N₂O₇Cl: 702.3072; found: 703.3127 (M+H) and 703.3124 (M+H).

### Example 940 1-(propanoyloxy)ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

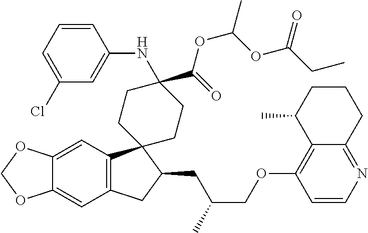 [SMILES: CCC(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

2,4,6-Trimethyl-1,3,5-trioxane (1.44 mL, 10.8 mml) and NaI (5130 mg, 34.2 mmol) was dissolved in DCM (100 mL), then propanoyl chloride (2.49 mL, 28.5 mmol) was added. The reaction mixture was stirred at rt overnight. The reaction mixture was filtered, and the filtrate was concentrated under reduced pressure resulting 1-iodoethyl propanoate as a racemate. Example 840 (100 mg, 0.162 mmol) and Cs₂CO₃ (316.7 mg, 0.9721 mmol) were suspended in dry DMF (3.24 mL), then 1-iodoethyl propanoate (184.7 mg, 0.8101 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 0.1 V/V % aq. TFA solution and MeCN as eluents resulting Example 940 as a mixture of diastereoisomers and as TFA salt. HRMS calculated for C₄₁H₄₉N₂O₇Cl: 716.3228; found: 717.3284 and 717.3282 (M+H).

### Example 941 1-[(methoxyacetyl)oxy]ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

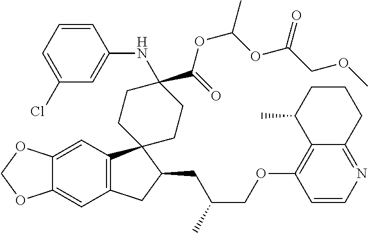 [SMILES: COCC(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

2,4,6-Trimethyl-1,3,5-trioxane (0.233 mL, 1.751 mml) and NaI (828.7 mg, 5.529 mmol) was dissolved in DCM (22 mL), then 2-methoxyacetyl chloride (0.421 mL, 4.607 mmol) was added.

The reaction mixture was stirred at rt overnight in darkness. The reaction mixture was filtered, and the filtrate was concentrated under reduced pressure resulting 1-iodoethyl methoxyacetate as a racemate. Example 840 (100 mg, 0.162 mmol) and Cs₂CO₃ (264.0 mg, 0.8101 mmol) were suspended in dry DMF (3.24 mL), then 1-iodoethyl methoxyacetate (197.7 mg, 0.8101 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 0.1 V/V % aq. TFA solution and MeCN as eluents resulting Example 941 as a mixture of diastereoisomers and as TFA salt. ¹H NMR (500 MHz, DMSO-d₆) δ ppm: 8.60 (d, 1H), 7.42 (d, 1H), 7.06/7.05 (t, 1H), 6.90 (m, 1H), 6.85/6.84 (s/s, 1H), 6.77 (s, 1H), 6.59 (t, 1H), 6.58 (dm, 1H), 6.48 (dm, 1H), 6.32/6.31 (br s/br s, 1H), 5.95 (m, 2H), 4.21/4.16 (dd+dd, 2H), 4.05/3.97/3.93 (d+d/d, 2H), 3.25 (s, 3H), 3.09 (m, 1H), 3.00-1.26 (m, 16H), 2.92/2.44 (m+dd, 2H), 2.19 (m, 1H), 2.04 (m, 1H), 1.38/1.35 (d/d, 3H), 1.07 (d, 3H), 1.06 (d, 3H). HRMS calculated for C₄₁H₄₉N₂O₈Cl: 732.3177; found: 733.3252 (M+H).

### Example 942 [(2,2-dimethylpropanoyl)oxy]methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

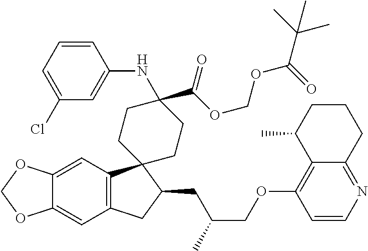 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCOC(=O)C(C)(C)C)CC2)OCO3]

Example 840 (100 mg, 0.162 mmol), NaI (36.4 mg, 0.243 mmol), chloromethyl 2,2-dimethylpropanoate (0.025 mL, 0.1701 mmol) were suspended in DMF (1.62 mL). To the stirred mixture Cs₂CO₃ (63.4 mg, 0.1944 mmol) was added. The reaction mixture was stirred at rt under N₂ atmosphere until no further conversion was observed. The product was isolated and purified by prep RP-HPLC using 0.1% aq. TFA solution and MeCN as eluents resulting Example 942 as TFA salt. HRMS calculated for C₄₂H₅₁N₂O₇Cl: 730.3385; found: 731.3436 (M+H).

### Example 943 1-[(methoxycarbonyl)oxy]ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

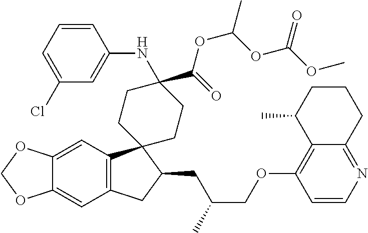 [SMILES: COC(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Dry MeOH (0.425 mL, 10.5 mmol) and pyridine (0.930 mL, 11.5 mmol) were dissolved in dry DCM (13 mL). The solution was cooled to −78° C., then 1-chloroethyl carbonochloridate (0.873 mL, 10.0 mmol) was added. The reaction mixture was stirred at −78° C. for 3 hours resulting 1-chloroethyl methyl carbonate as DCM solution. Example 840 (100 mg, 0.162 mmol) and Cs₂CO₃ (527.9 mg, 1.620 mmol) were suspended in DMF (3.24 mL). To the stirred mixture solution of 1-chloroethyl methyl carbonate in DCM (2.0 mL, 1.30 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 943 as a mixture of diastereoisomers. ¹H NMR (500 MHz, DMSO-d₆) δ ppm: 8.14 (d, 1H), 7.05/7.04 (t/t, 1H), 6.85/6.82 (s/s, 1H), 6.77 (s, 1H), 6.76 (d, 1H), 6.73 (m, 1H), 6.59 (m, 1H), 6.58 (m, 1H), 6.48 (m, 1H), 6.31 (s, 1H), 5.95 (m, 2H), 3.86 (m, 2H), 3.71 (s, 3H), 3.04 (m, 1H), 2.90/2.43 (d+d, 2H), 2.76/2.65 (dm+m, 2H), 2.48-1.23 (m, 14H), 2.16 (m, 1H), 1.96 (m, 1H), 1.38/1.37 (d/d, 3H), 1.05 (d, 3H), 1.04 (d, 3H). HRMS calculated for C₄₀H₄₇N₂O₈Cl: 718.3021; found: 719.3071 (M+H) and 719.3070 (M+H).

### Example 944 1-({[(propan-2-yl)oxy]carbonyl}oxy)ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

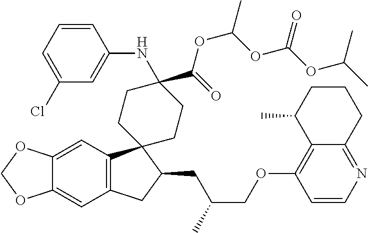 [SMILES: CC(C)OC(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Dry propan-2-ol (0.804 mL, 10.5 mmol) and pyridine (0.930 mL, 11.5 mmol) were dissolved in dry DCM (13 mL). The solution was cooled to −78° C., then 1-chloroethyl carbonochloridate (0.873 mL, 10.0 mmol) was added. The reaction mixture was stirred at −78° C. for 3 hours resulting 1-chloroethyl propan-2-yl carbonate as DCM solution. Example 840 (100 mg, 0.162 mmol) and Cs₂CO₃ (527.9 mg, 1.620 mmol) were suspended in DMF (3.24 mL). To the stirred mixture solution of 1-chloroethyl propan-2-yl carbonate in DCM (2.0 mL, 1.30 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 944 as a mixture of diastereoisomers. ¹H NMR (500 MHz, DMSO-d₆) δ ppm: 8.15 (d, 1H), 7.05/7.04 (t/t, 1H), 6.82/6.80 (s/s, 1H), 6.77 (d, 1H), 6.77/6.76 (s/s, 1H), 6.73/6.72 (q/q, 1H), 6.60/6.59 (t/t, 1H), 6.58/6.57 (dd/dd, 1H), 6.50/6.48 (dd/dd, 1H), 6.31/6.30 (s/s, 1H), 5.95/5.94 (m/m, 2H), 4.75 (m, 1H), 3.90/3.88/3.86/3.84 (dd+dd/dd+dd, 2H), 3.04 (m, 1H), 2.91/2.90/2.43/2.43 (dd+dd/dd+dd, 2H), 2.76/2.65 (m+m, 2H), 2.45-1.24 (m, 8H), 2.19/2.17 (m/m, 1H), 1.96 (m, 1H), 1.80/1.73 (m+m, 2H), 1.68/1.60 (m+m, 2H), 1.45/1.28 (t+t, 2H), 1.36/1.34 (d/d, 3H), 1.21/1.16/1.14 (d/d+d, 6H), 1.04 (d, 3H), 1.03 (d, 3H). HRMS calculated for C₄₂H₅₁N₂O₈Cl: 746.3334; found: 747.3391 (M+H) and 747.3394 (M+H).

### Example 945 1-{[(cyclopentyloxy)carbonyl]oxy}ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

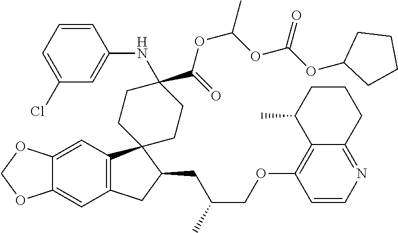 [SMILES: CC(OC(=O)OC1CCCC1)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Dry cyclopentanol (0.953 mL, 10.5 mmol) and pyridine (0.930 mL, 11.5 mmol) were dissolved in dry DCM (13 mL). The solution was cooled to −78° C., then 1-chloroethyl carbonochloridate (0.873 mL, 10.0 mmol) was added. The reaction mixture was stirred at −78° C. for 3 hours resulting 1-chloroethyl cyclopentyl carbonate as DCM solution. Example 840 (100 mg, 0.162 mmol) and Cs₂CO₃ (527.9 mg, 1.620 mmol) were suspended in DMF (3.24 mL). To the stirred mixture solution of 1-chloroethyl cyclopentyl carbonate in DCM (2.0 mL, 1.30 mmol) was added dropwise at rt. The reaction mixture was stirred at rt for 10 hours, then another portion of 1-chloroethyl cyclopentyl carbonate (2.0 mL, 1.30 mmol) was added. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 945 as a mixture of diastereoisomers. ¹H NMR (500 MHz, DMSO-d₆) δ ppm: 8.14 (d, 1H), 7.04 (t, 1H), 6.82/6.81 (s/s, 1H), 6.77 (s, 1H), 6.76 (d, 1H), 6.72 (m, 1H), 6.59 (m, 1H), 6.58 (m, 1H), 6.49 (m, 1H), 6.30/6.29 (s/s, 1H), 5.94 (m, 2H), 4.98 (m, 1H), 3.92-3.80 (m, 2H), 3.03 (m, 1H), 2.90/2.43 (dm+dd, 2H), 2.76/2.65 (dm+m, 2H), 2.47-1.23 (m, 22H), 2.17 (m, 1H), 1.95 (m, 1H), 1.36/1.34 (d/d, 3H), 1.04 (d, 3H), 1.03 (d, 3H). HRMS calculated for C₄₄H₅₃N₂O₈Cl: 772.3491; found: 773.3569 (M+H) and 773.3582 (M+H).

### Example 946 [(methoxycarbonyl)oxy]methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

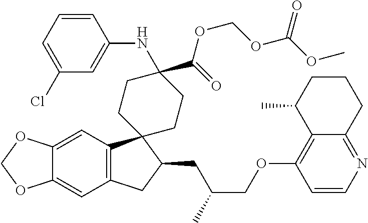 [SMILES: COC(=O)OCOC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Dry MeOH (0.425 mL, 10.5 mmol) and pyridine (0.930 mL, 11.5 mmol) were dissolved in dry DCM (13 mL). The solution was cooled to −78° C., then chloromethyl carbonochloridate (0.889 mL, 10.0 mmol) was added. The reaction mixture was stirred at −78° C. for 3 hours resulting chloromethyl methyl carbonate as DCM solution. Example 840 (100 mg, 0.162 mmol) and Cs₂CO₃ (527.9 mg, 1.620 mmol) were suspended in DMF (3.24 mL). To the stirred mixture solution of chloromethyl methyl carbonate in DCM (2.0 mL, 1.30 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 946. ¹H NMR (500 MHz, DMSO-d₆) δ ppm: 8.14 (d, 1H), 7.03 (t, 1H), 6.84 (s, 1H), 6.77 (s, 1H), 6.76 (d, 1H), 6.58 (m, 1H), 6.57 (m, 1H), 6.44 (dm, 1H), 6.35 (s, 1H), 5.95/5.94 (d+d, 2H), 5.76/5.74 (d+d, 2H), 3.89/3.84 (dd+dd, 2H), 3.65 (s, 3H), 3.04 (m, 1H), 2.90/2.43 (dd+dd, 2H), 2.76/2.65 (dm+m, 2H), 2.45-1.25 (m, 14H), 2.15 (m, 1H), 1.96 (m, 1H), 1.05 (d, 3H), 1.03 (d, 3H). HRMS calculated for C₃₉H₄₅N₂O₈Cl: 704.2864; found: 705.2931 (M+H).

### Example 947 1-{[(2-methoxyethyl)carbamoyl]oxy}ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

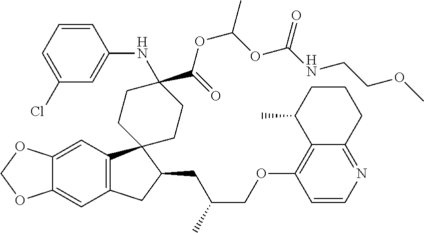 [SMILES: COCCNC(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

2-Methoxyethanamine (0.913 mL, 10.5 mmol) and pyridine (0.930 mL, 11.5 mmol) were dissolved in dry DCM (13 mL). The solution was cooled to −78° C., then 1-chloroethyl carbonochloridate (0.873 mL, 10.0 mmol) was added. The reaction mixture was stirred at −78° C. for 3 hours resulting 1-chloroethyl (2-methoxyethyl)carbamate as DCM solution. Example 840 (100 mg, 0.162 mmol) and Cs₂CO₃ (527.9 mg, 1.620 mmol) were suspended in DMF (3.24 mL). To the stirred mixture solution of 1-chloroethyl (2-methoxyethyl)carbamate in DCM (2.0 mL, 1.30 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 947 as a mixture of diastereoisomers. ¹H NMR (500 MHz, DMSO-d₆) δ ppm: 8.14/8.13 (d/d, 1H), 7.55/7.52 (t/t, 1H), 7.04 (t, 1H), 6.83/6.82 (s/s, 1H), 6.76 (d, 1H), 6.76/6.75 (s/s, 1H), 6.75 (s/s, 1H), 6.61/6.60 (t/t, 1H), 6.57 (dd, 1H), 6.50 (dd, 1H), 6.28/6.24 (s/s, 1H), 5.95/5.94/5.93/5.92 (m+m/m+m, 2H), 3.88/3.84 (dd+dd, 2H), 3.29/3.25 (m/m, 2H), 3.18/3.17 (s/s, 3H), 3.14/3.09 (m/m, 2H), 3.03 (m, 1H), 2.90/2.43 (dd+dd, 2H), 2.75/2.65 (m+m, 2H), 2.45-1.24 (m, 8H), 2.21/2.19 (m/m, 1H), 1.96 (m, 1H), 1.80/1.74 (m+m, 2H), 1.67/1.60 (m+m, 2H), 1.43/1.27 (t+t, 2H), 1.32/1.30 (d/d, 3H), 1.05/1.03 (d/d, 3H), 1.04/1.02 (d/d, 3H). HRMS calculated for C₄₂H₅₂N₃O₈Cl: 761.3443; found: 762.351 (M+H).

### Example 948 1-({(1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carbonyl}oxy)ethyl morpholine-4-carboxylate

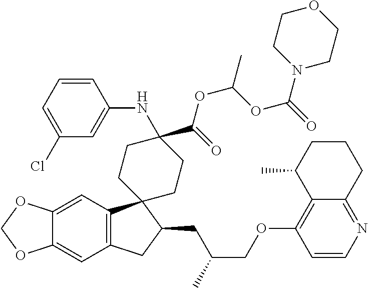 [SMILES: CC(OC(=O)N1CCOCC1)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Morpholine (0.918 mL, 10.5 mmol) and pyridine (0.930 mL, 11.5 mmol) were dissolved in dry DCM (13 mL). The solution was cooled to −78° C., then 1-chloroethyl carbonochloridate (0.873 mL, 10.0 mmol) was added. The reaction mixture was stirred at −78° C. for 3 hours, them it was allowed to warm to rt resulting 1-chloroethyl morpholine-4-carboxylate as DCM solution. Example 840 (100 mg, 0.162 mmol) and Cs₂CO₃ (527.9 mg, 1.620 mmol) were suspended in DMF (3.24 mL). To the stirred mixture solution of 1-chloroethyl morpholine-4-carboxylate in DCM (2.0 mL, 1.30 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 948 as a mixture of diastereoisomers. ¹H NMR (500 MHz, DMSO-d₆) δ ppm: 8.14 (d, 1H), 7.05/7.04 (t/t, 1H), 6.83 (s, 1H), 6.78/6.77 (q/q, 1H), 6.76/6.75 (s/s, 1H), 6.76 (d, 1H), 6.60/6.59 (t/t, 1H), 6.58/6.57 (dd/dd, 1H), 6.51/6.49 (dd/dd, 1H), 6.30/6.29 (s/s, 1H), 5.95/5.94 (m/m, 2H), 3.89/3.88/3.85/3.83 (dd+dd/dd+dd, 2H), 3.59-3.38 (br, 4H), 3.35-3.26 (br, 4H), 3.03 (m, 1H), 2.91/2.90/2.43/2.42 (dd+dd/dd+dd, 2H), 2.76/2.65 (m+m, 2H), 2.44-1.22 (m, 8H), 2.18/2.15 (m/m, 1H), 1.97/1.95 (m/m, 1H), 1.79/1.73 (m+m, 2H), 1.67/1.60 (m+m, 2H), 1.45/1.43/1.29/1.26 (t+t/t+t, 2H), 1.39/1.38 (d/d, 3H), 1.04/1.03 (d/d, 3H), 1.03 (d, 3H). HRMS calculated for C₄₃H₅₂N₃O₈Cl: 773.3443; found: 774.3491 (M+H) and 774.3496 (M+H).

### Example 949 1-[(diethylcarbamoyl)oxy]ethyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

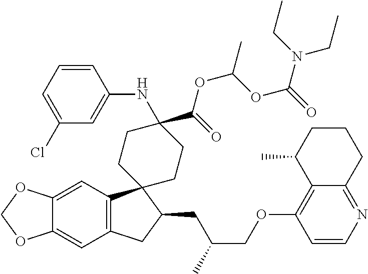 [SMILES: CCN(CC)C(=O)OC(C)OC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

N-ethylethanamine (1.086 mL, 10.5 mmol) and pyridine (0.930 mL, 11.5 mmol) were dissolved in dry DCM (13 mL). The solution was cooled to −78° C., then 1-chloroethyl carbonochloridate (0.873 mL, 10.0 mmol) was added. The reaction mixture was stirred at −78° C. for 3 hours resulting 1-chloroethyl diethylcarbamate as DCM solution. Example 840 (100 mg, 0.162 mmol) and Cs₂CO₃ (527.9 mg, 1.620 mmol) were suspended in DMF (3.24 mL). To the stirred mixture solution of 1-chloroethyl diethylcarbamate in DCM (2.0 mL, 1.30 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 949 as a mixture of diastereoisomers. ¹H NMR (500 MHz, DMSO-d₆) δ ppm: 8.13 (d, 1H), 7.04/7.03 (t/t, 1H), 6.83/6.81 (s/s, 1H), 6.78/6.77 (q/q, 1H), 6.76/6.75 (s/s, 1H), 6.76 (d, 1H), 6.61/6.60 (t/t, 1H), 6.57 (dd, 1H), 6.50/6.49 (dd/dd, 1H), 6.28/6.26 (s/s, 1H), 5.94 (m, 2H), 3.88/3.84 (dd+dd, 2H), 3.22/3.15 (m/m, 4H), 3.03 (m, 1H), 2.90/2.43 (dd+dd, 2H), 2.76/2.65 (m+m, 2H), 2.44-1.38 (m, 8H), 2.18/2.16 (m, 1H), 1.95 (m, 1H), 1.79/1.74 (m+m, 2H), 1.67/1.59 (m+m, 2H), 1.43/1.27 (t+t, 2H), 1.37/1.36 (d/d, 3H), 1.03 (d, 3H), 1.03 (d, 3H), 1.00/0.98 (t/t, 6H). HRMS calculated for C₄₃H₅₄N₃O₇Cl: 759.3651; found: 760.3713 (M+H) and 760.3704 (M+H).

### Example 950 [(diethylcarbamoyl)oxy]methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

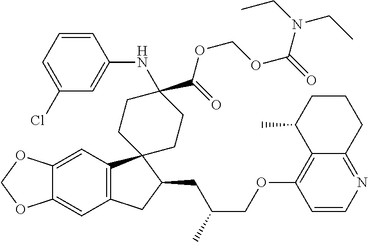 [SMILES: CCN(CC)C(=O)OCOC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

N-ethylethanamine (1.086 mL, 10.5 mmol) and pyridine (0.930 mL, 11.5 mmol) were dissolved in dry DCM (13 mL). The solution was cooled to −78° C., then chloromethyl N,N-diethylcarbamate (0.889 mL, 10.0 mmol) was added. The reaction mixture was stirred at −78° C. for 3 hours resulting chloromethyl N,N-diethylcarbamate as DCM solution. Example 840 (200 mg, 0.3241 mmol) and Cs₂CO₃ (1055.9 mg, 3.2406 mmol) were suspended in DMF (6.481 mL). To the stirred mixture solution of chloromethyl N,N-diethylcarbamate in DCM (4.0 mL, 2.60 mmol) was added dropwise at rt. The reaction mixture was stirred at rt until no further conversion was observed. The reaction mixture was poured onto ice/water mixture, then brine was added. The mixture was extracted with EtOAc. The combined organic layers were dried over MgSO₄, filtered and the filtrate was concentrated under reduced pressure. The crude product was purified by prep RP-HPLC using 25 mM aq. NH₄HCO₃ solution and MeCN as eluents resulting Example 950. ¹H NMR (500 MHz, DMSO-d₆) δ ppm: 8.14 (d, 1H), 7.02 (t, 1H), 6.81 (s, 1H), 6.76 (s, 1H), 6.76 (d, 1H), 6.60 (t, 1H), 6.56 (dm, 1H), 6.46 (dm, 1H), 6.34 (s, 1H), 5.96/5.94 (d+d, 2H), 5.76/5.74 (d+d, 2H), 3.88/3.84 (dd+dd, 2H), 3.17/3.11 (q, 4H), 3.03 (m, 1H), 2.90/2.43 (dd+dd, 2H), 2.75/2.65 (m+m, 2H), 2.47-1.31 (m, 12H), 2.17 (m, 1H), 1.96 (m, 1H), 1.44/1.28 (m+m, 2H), 1.03 (d, 3H), 1.03 (d, 3H), 0.97 (t, 6H). HRMS calculated for C₄₂H₅₂N₃O₇Cl: 745.3494; found: 746.3566 (M+H).

### Example 951 [(di-tert-butoxyphosphoryl)oxy]methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

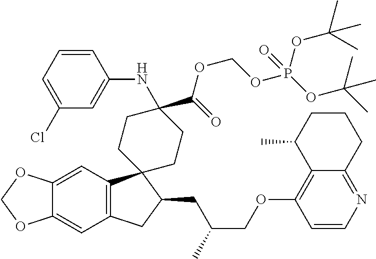 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCOP(=O)(OC(C)(C)C)OC(C)(C)C)CC2)OCO3]

Example 840 (100 mg, 0.162 mmol) was dissolved dry DMF (3.240 mL), then NaI (36.4 mg, 0.243 mmol), Cs₂CO₃ (158.4 mg, 0.4861 mmol) and ditert-butyl chloromethyl phosphate (62.9 mg, 0.243 mmol) were added at rt. The reaction mixture was stirred at rt until no further conversion was observed. The reaction mixture was diluted with water and brine, and was extracted with EtOAc. The combined organic layers were washed with brine, dried over Na₂SO₄, filtered and the filtrate was concentrated under reduced pressure. The crude product was purified by prep RP-HPLC using 25 mM aq. NH₄HCO₃ solution and MeCN as eluents resulting Example 951. ¹H NMR (500 MHz, DMSO-d₆) δ ppm: 8.14 (d, 1H), 7.03 (t, 1H), 6.86 (s, 1H), 6.77 (s, 1H), 6.76 (d, 1H), 6.62 (t, 1H), 6.57 (dd, 1H), 6.48 (dd, 1H), 6.35 (s, 1H), 5.95/5.93 (d+d, 2H), 5.58/5.55 (t+t, 2H), 3.89/3.85 (dd+dd, 2H), 3.04 (m, 1H), 2.90/2.43 (dd+dd, 2H), 2.76/2.65 (m+m, 2H), 2.48-1.34 (m, 8H), 2.17 (m, 1H), 1.97 (m, 1H), 1.80/1.74 (m+m, 2H), 1.68/1.60 (m+m, 2H), 1.47/1.30 (t+t, 2H), 1.39/1.38 (s+s, 18H), 1.04 (d, 3H), 1.03 (d, 3H). HRMS calculated for C₄₅H₆₀N₂O₉PCl: 838.3725; found: 839.3795 (M+H).

### Example 952 (phosphonooxy)methyl (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

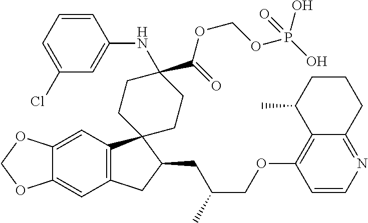 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCOP(=O)(O)O)CC2)OCO3]

Example 951 (43 mg, 0.0512 mmol) was dissolved in DCM (5.0 mL). The solution was cooled to −15° C., then TFA (0.70 mL, 9.0 mmol) was added. The reaction mixture was stirred at −15° C. until no further conversion was observed. The reaction mixture was concentrated under reduced pressure at rt, then sat. aq. NaHCO₃ solution was added to the residue. The crude product was purified by prep RP-HPLC using 25 mM aq. NH₄HCO₃ solution and MeCN as eluents resulting Example 952. HRMS calculated for C₃₇H₄₄N₂O₉PCl: 726.2473; found: 727.2540 (M+H).

### Example 953 3-({(1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carbonyl}oxy)propane-1-sulfonic acid

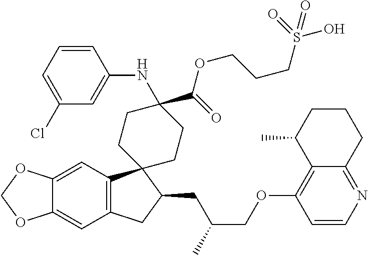 [SMILES: C[C@@H](COc1ccnc2c1[C@H](C)CCC2)C[C@H]1Cc2cc3c(cc2[C@]12CC[C@@](Nc1cccc(Cl)c1)(C(=O)OCCCS(=O)(=O)O)CC2)OCO3]

Example 840 (100 mg, 0.162 mmol) was dissolved in DMF (3.240 mL), then oxathiolane 2,2-dioxide (29.7 mg, 0.243 mmol) and Cs₂CO₃ (25.8 mg, 0.243 mmol) were added. The reaction mixture was stirred at 80° C. under N₂ atmosphere until no further conversion was observed. The reaction mixture was diluted with water, and the pH was set to 6 with 1 M aq. HCl solution. The reaction mixture was extracted with EtOAc. The combined organic layers were washed with brine, dried over Na₂SO₄, filtered and the filtrate was concentrated under reduced pressure. The crude product was purified by flash chromatography using heptane and EtOAc, then EtOAc and MeOH as eluents to obtain Example 953. HRMS calculated for C₃₉H₄₇N₂O₈SCl: 738.2742; found: 739.2823 (M+H).

### Example 954 (1r,4S,6′S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxamide

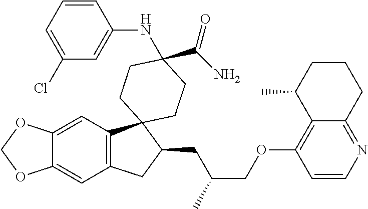 [SMILES: C[C@@H](COc1ccnc2c1[C@H](C)CCC2)C[C@H]1Cc2cc3c(cc2[C@]12CC[C@@](Nc1cccc(Cl)c1)(C(N)=O)CC2)OCO3]

Example 840 (60 mg, 0.097 mmol) was dissolved in dry DMF (970 μL). HOBt×H₂0 (22 mg, 0.15 mmol) and EDC×HCl (26 mg, 0.14 mmol) were added to the mixture and stirred at rt under N₂ for 1 hour. Aq. NH₃ solution (37 μL, 0.51 mmol) was added and the mixture was stirred at rt under N₂ until no further conversion was observed. The crude product was purified via prep RP-HPLC using 25 mM aq. NH₄HCO₃ solution and MeCN as eluents resulting Example 954. ¹H NMR (500 MHz, DMSO-d₆) δ ppm: 8.16 (d, 1H), 7.31/7.12 (s+s, 2H), 7.05 (t, 1H), 6.93 (s, 1H), 6.78 (d, 1H), 6.77 (s, 1H), 6.65 (t, 1H), 6.58 (dd, 1H), 6.55 (dd, 1H), 6.05 (s, 1H), 5.96/5.94 (d+d, 2H), 3.90/3.86 (dd+dd, 2H), 3.08 (m, 1H), 2.89/2.41 (dd+dd, 2H), 2.77/2.67 (m+m, 2H), 2.39-1.23 (m, 8H), 2.08 (m, 1H), 1.97 (m, 1H), 1.78/1.72 (m+m, 2H), 1.63/1.59 (m+m, 2H), 1.50/1.37 (dd+dd, 2H), 1.08 (d, 3H), 1.04 (d, 3H). HRMS calculated for C₃₆H₄₂N₃O₄Cl: 615.2864; found: 616.2933 (M+H).

### Example 955 (1r,4S,6′S)-4-(3-chloroanilino)-N-methyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxamide

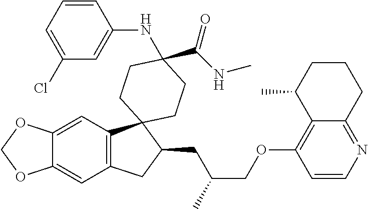 [SMILES: CNC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCO3]

Example 840 (60 mg, 0.097 mmol) was dissolved in dry DMF (970 μL). HOBt×H₂0 (22 mg, 0.15 mmol) and EDC×HCl (26 mg, 0.14 mmol) was added to the mixture and stirred at rt under N₂ for 1 hour. Methanamine (44 μL, 0.51 mmol) was added and the mixture was stirred at rt under N₂ until no further conversion was observed. The crude product was purified via prep RP-HPLC using 25 mM aq. NH₄HCO₃ solution and MeCN as eluents resulting Example 955. ¹H NMR (500 MHz, DMSO-d₆) δ ppm: 8.16 (d, 1H), 7.84 (q, 1H), 7.05 (t, 1H), 6.96 (s, 1H), 6.79 (d, 1H), 6.77 (s, 1H), 6.65 (t, 1H), 6.58 (dd, 1H), 6.47 (dd, 1H), 6.06 (s, 1H), 5.96/5.94 (d+d, 2H), 3.90/3.87 (dd+dd, 2H), 3.09 (m, 1H), 2.88/2.40 (dd+dd, 2H), 2.78/2.67 (m+m, 2H), 2.60 (d, 3H), 2.40-1.20 (m, 8H), 2.08 (m, 1H), 1.97 (m, 1H), 1.78/1.73 (m+m, 2H), 1.64/1.60 (m+m, 2H), 1.52/1.38 (t+t, 2H), 1.08 (d, 3H), 1.04 (d, 3H). HRMS calculated for C₃₇H₄₄N₃O₄Cl: 629.3021; found: 630.3097 (M+H).

### Example 956 (1r,4S,6′S)-4-(3-chloroanilino)-N,N-dimethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxamide

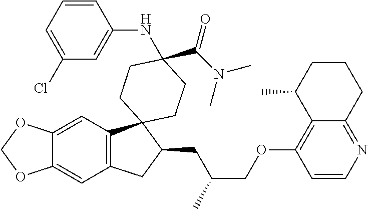 [SMILES: C[C@@H](COc1ccnc2c1[C@H](C)CCC2)C[C@H]1Cc2cc3c(cc2[C@]12CC[C@@](Nc1cccc(Cl)c1)(C(=O)N(C)C)CC2)OCO3]

Example 840 (100 mg, 0.16 mmol) and TEA (68 μL, 0.49 mmol) were dissolved in dry THF (1.6 mL) and cooled to 0° C. Ethyl carbonochloridate (33 μL, 0.34 mmol) was added to the mixture and stirred at 0° C. for 5 minutes. N-methylmethanamine (203 μL, 0.41 mmol) was added to the mixture and stirred at rt until no further conversion was observed. Water was added to the mixture and it was concentrated under reduced pressure. The crude product was purified via prep RP-HPLC using 25 mM aq. NH₄HCO₃ solution and MeCN as eluents resulting Example 956. ¹H NMR (500 MHz, DMSO-d₆) δ ppm: 8.16 (d, 1H), 7.03 (t, 1H), 6.88 (s, 1H), 6.79 (d, 1H), 6.77 (s, 1H), 6.56 (t, 1H), 6.52 (dd, 1H), 6.45 (dd, 1H), 6.39 (s, 1H), 5.95 (d, 2H), 3.92/3.87 (dd+dd, 2H), 3.10/2.83 (br s+br s, 6H), 3.06 (m, 1H), 2.89/2.42 (dd+dd, 2H), 2.77/2.66 (m+m, 2H), 2.38-1.23 (m, 8H), 2.09 (m, 1H), 1.96 (m, 1H), 1.79/1.74 (m+m, 2H), 1.67/1.63 (m+m, 2H), 1.50/1.36 (t+t, 2H), 1.08 (d, 3H), 1.05 (d, 3H). HRMS calculated for C₃₈H₄₆N₃O₄Cl: 643.3177; found: 644.3253 (M+H).

### Example 957 (1r,4S,6′S)-4-(3-chloroanilino)-N-(methanesulfonyl)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxamide

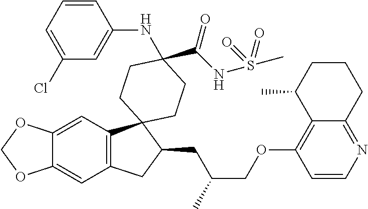 [SMILES: C[C@@H](COc1ccnc2c1[C@H](C)CCC2)C[C@H]1Cc2cc3c(cc2[C@]12CC[C@@](Nc1cccc(Cl)c1)(C(=O)NS(C)(=O)=O)CC2)OCO3]

Example 840 (60 mg, 0.097 mmol) was dissolved in dry DCM (1.5 mL). EDC×HCl (37 mg, 0.19 mmol), DMAP (24 mg, 0.19 mmol) and methanesulfonamide (19 μL, 0.097 mmol) were added to the mixture and stirred at 40° C. under N₂ until no further conversion was observed. The reaction mixture was concentrated under reduced pressure and the crude product was purified via prep RP-HPLC using 25 mM aq. NH₄HCO₃ solution and MeCN as eluents resulting Example 957. ¹H NMR (500 MHz, DMSO-d₆) δ ppm: 11.93 (br s, 1H), 8.19 (d, 1H), 7.06 (t, 1H), 6.86 (s, 1H), 6.83 (d, 1H), 6.77 (s, 1H), 6.68 (t, 1H), 6.58 (dd, 1H), 6.50 (dd, 1H), 5.96/5.94 (d+d, 2H), 5.95 (br s, 1H), 3.93/3.88 (dd+dd, 2H), 3.08 (s, 3H), 3.06 (m, 1H), 2.90/2.42 (dd+dd, 2H), 2.79/2.68 (m+m, 2H), 2.39-1.34 (m, 8H), 2.13 (m, 1H), 1.97 (m, 1H), 1.78/1.73 (m+m, 2H), 1.64/1.60 (m+m, 2H), 1.48/1.36 (t+t, 2H), 1.07 (d, 3H), 1.05 (d, 3H). HRMS calculated for C₃₇H₄₄N₃O₆SCl: 693.2639; found: 694.2710 (M+H).

### Example 958 (1r,4S,6′S)-4-(3-chloroanilino)-6′-[(2R)-2-methyl-3-{[(5R)-5-methyl-5,6,7,8-tetrahydroquinolin-4-yl]oxy}propyl]-N-(trifluoromethanesulfonyl)-6′,7′-dihydro-2′H-spiro[cyclohexane-1,5′-indeno[5,6-d][1,3]dioxole]-4-carboxamide

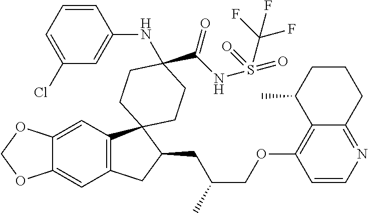 [SMILES: C[C@@H](COc1ccnc2c1[C@H](C)CCC2)C[C@H]1Cc2cc3c(cc2[C@]12CC[C@@](Nc1cccc(Cl)c1)(C(=O)NS(=O)(=O)C(F)(F)F)CC2)OCO3]

Example 840 (120 mg, 0.19 mmol) was dissolved in dry DCM (2.9 mL). EDC×HCl (75 mg, 0.39 mmol), DMAP (48 mg, 0.39 mmol) and 1,1,1-trifluoromethanesulfonamide (61 μL, 0.19 mmol) were added to the mixture and stirred at 40° C. under N₂ until no further conversion was observed. Then it was diluted with brine and extracted with DCM. The combined organic layer was washed with brine, water, then dried over MgSO₄, filtered and the filtrate was concentrated under reduced pressure. The crude product was purified via prep RP-HPLC using 25 mM aq. NH₄HCO₃ solution and MeCN as eluents resulting Example 958. ¹H NMR (500 MHz, DMSO-d₆) δ ppm: 14.46 (br s, 1H), 8.55 (d, 1H), 7.38 (d, 1H), 6.93 (t, 1H), 6.85 (s, 1H), 6.74 (s, 1H), 6.61 (t, 1H), 6.51 (dm, 1H), 6.42 (dm, 1H), 5.94/5.93 (d+d, 2H), 5.93 (br s, 1H), 4.21/4.14 (dd+dd, 2H), 3.11 (m, 1H), 2.94/2.86 (m+m, 2H), 2.90/2.42 (dd+dd, 2H), 2.47-1.30 (m, 12H), 2.13 (m, 1H), 2.03 (m, 1H), 1.45/1.36 (m+m, 2H), 1.10 (d, 3H), 1.06 (d, 3H). HRMS calculated for C₃₇H₄₁N₃O₆F₃SCl: 747.2357; found: 748.2433 (M+H).

### Example 959

### Example 959A methyl (1r,4S,6′S)-4-(3-chloroanilino)-6′-[(2R)-2-methyl-3-{[(5R,8R)-5-methyl-8-{[tri(propan-2-yl)silyl]oxy}-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate

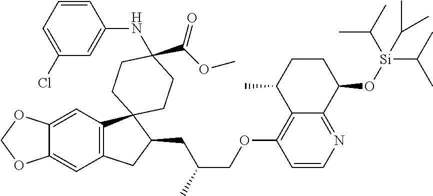 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CC[C@H]2O[Si](C(C)C)(C(C)C)C(C)C)OCO3]

Using General procedure 30a and Example 839E as the appropriate indane and Example 771B as the appropriate alcohol, Example 959A was obtained. HRMS calculated for C₄₆H₆₃ClN₂O₆Si: 802.4144; found: 803.4222 (M+H).

### Example 959B methyl (1r,4S,6′S)-4-(3-chloroanilino)-6′-[(2R)-3-{[(5R,8R)-8-hydroxy-5-methyl-5,6,7,8-tetrahydroquinolin-4-yl]oxy}-2-methylpropyl]-6′,7′-dihydro-2′H-spiro[cyclohexane-1,5′-indeno[5,6-d[1,3]dioxole]-4-carboxylate

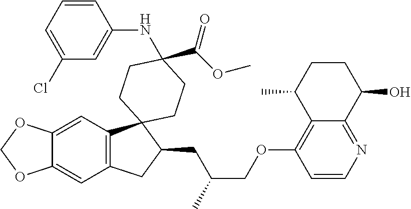 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CC[C@H]2O)OCO3]

Using General procedure 29 (performing the reaction at 50° C. instead of rt) and Example 959A as the appropriate silyl derivative, Example 959B was obtained. ¹H NMR (500 MHz, DMSO-d₆) δ ppm: 8.25 (d, 1H), 7.04 (t, 1H), 6.89 (s, 1H), 6.85 (d, 1H), 6.78 (s, 1H), 6.57 (t, 1H), 6.55 (dm, 1H), 6.45 (dd, 1H), 6.31 (s, 1H), 5.95/5.94 (d+d, 2H), 4.92 (d, 1H), 4.44 (q, 1H), 3.92/3.85 (dd+dd, 2H), 3.65 (s, 3H), 3.02 (m, 1H), 2.90/2.43 (dd+dd, 2H), 2.47-1.25 (m, 10H), 2.14 (m, 1H), 2.06/1.38 (m+dm, 2H), 1.98 (m, 1H), 1.95/1.73 (m+m, 2H), 1.04 (d, 3H), 1.02 (d, 3H). HRMS calculated for C₃₇H₄₃N₂O₆Cl: 646.2810; found: 647.2886 (M+H).

### Example 959 (1r,4S,6′S)-4-(3-chloroanilino)-6′-[(2R)-3-{[(5R,8R)-8-hydroxy-5-methyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid

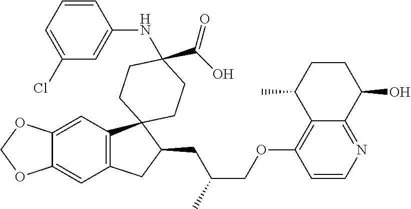 [SMILES: C[C@@H](COc1ccnc2c1[C@H](C)CC[C@H]2O)C[C@H]1Cc2cc3c(cc2[C@]12CC[C@@](Nc1cccc(Cl)c1)(C(=O)O)CC2)OCO3]

Using General procedure 33a and Example 959B as the appropriate ester, Example 959 was obtained. ¹H NMR (500 MHz, DMSO-d₆) δ ppm: 12.64 (br s, 1H), 8.26 (d, 1H), 7.04 (t, 1H), 6.87 (d, 1H), 6.87 (s, 1H), 6.79 (s, 1H), 6.60 (t, 1H), 6.55 (dm, 1H), 6.45 (dd, 1H), 6.24 (br s, 1H), 5.96/5.95 (d+d, 2H), 4.95 (d, 1H), 4.45 (q, 1H), 3.93/3.86 (dd+dd, 2H), 3.04 (m, 1H), 2.91/2.44 (dd+dd, 2H), 2.46-1.26 (m, 10H), 2.15 (m, 1H), 2.08/1.39 (m+m, 2H), 1.99 (m, 1H), 1.96/1.74 (m+m, 2H), 1.05 (d, 3H), 1.03 (d, 3H). HRMS calculated for C₃₆H₄₁N₂O₆Cl: 632.2653; found: 633.2730 (M+H).

### Example 960 (1r,4S,6′S)-4-(3-chloroanilino)-6′-[(2R)-3-{[(5R,8R)-5,8-dimethyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid

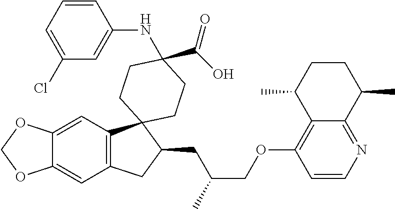 [SMILES: C[C@@H](COc1ccnc2c1[C@H](C)CC[C@H]2C)C[C@H]1Cc2cc3c(cc2[C@]12CC[C@@](Nc1cccc(Cl)c1)(C(=O)O)CC2)OCO3]

Using General procedure 32 and Example 839E as the appropriate indane and Example 658G as the appropriate alcohol, Example 960 was obtained. ¹H NMR (500 MHz, DMSO-d₆) δ ppm: 12.71 (br s, 1H), 8.18 (d, 1H), 7.03 (t, 1H), 6.86 (s, 1H), 6.76 (s, 1H), 6.75 (d, 1H), 6.60 (t, 1H), 6.53 (dd, 1H), 6.52 (dd, 1H), 6.25 (br s, 1H), 5.94 (d, 2H), 3.89/3.83 (dd+dd, 2H), 3.04 (m, 1H), 2.90/2.43 (dd+dd, 2H), 2.85 (m, 1H), 2.41-1.41 (m, 8H), 2.14 (m, 1H), 2.02/1.50 (td+dd, 2H), 1.96 (m, 1H), 1.86/1.44 (td+dd, 2H), 1.45/1.31 (t+t, 2H), 1.17 (d, 3H), 1.04 (d, 3H), 1.04 (d, 3H). HRMS calculated for C₃₇H₄₃N₂O₅Cl: 630.2861; found: 631.2936 (M+H).

### Example 961 (1r,4S,6′S)-4-(3-chloroanilino)-6′-[(2R)-3-{[(5R,8R)-8-fluoro-5-methyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid

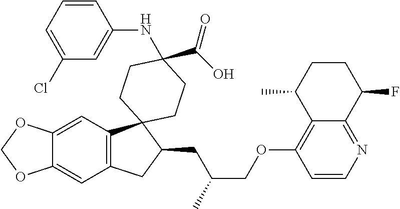 [SMILES: C[C@@H](COc1ccnc2c1[C@H](C)CC[C@H]2F)C[C@H]1Cc2cc3c(cc2[C@]12CC[C@@](Nc1cccc(Cl)c1)(C(=O)O)CC2)OCO3]

Using General procedure 32 and Example 839E as the appropriate indane and Example 772B as the appropriate alcohol, Example 961 was obtained. ¹H NMR (500 MHz, DMSO-d₆) δ ppm: 8.33 (d, 1H), 7.02 (t, 1H), 6.99 (d, 1H), 6.86 (s, 1H), 6.77 (s, 1H), 6.60 (t, 1H), 6.52 (m, 2H), 6.22 (br s, 1H), 5.95/5.94 (s+s, 2H), 5.36 (d, 1H), 3.95/3.90 (dd+dd, 2H), 3.12 (m, 1H), 2.90/2.43 (dd+dd, 2H), 2.43-1.27 (m, 10H), 2.43-1.27 (m, 2H), 2.14 (m, 1H), 1.99 (m, 1H), 1.96/1.53 (m+m, 2H), 1.05 (d, 3H), 1.03 (d, 3H). HRMS calculated for C₃₆H₄₀N₂05FCl: 634.2610; found: 635.2684 (M+H).

### Example 962

### Example 962A methyl (1r,4S,7′S)-4-(3-chloroanilino)-7′-[(2R)-2-methyl-3-{[(5R,8R)-5-methyl-8-{[tri(propan-2-yl)silyl]oxy}-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

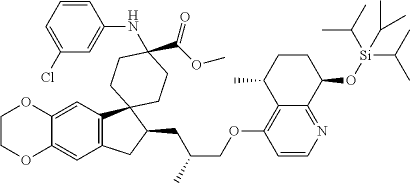 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CC[C@H]2O[Si](C(C)C)(C(C)C)C(C)C)OCCO3]

Using General procedure 30a and Example 8380 as the appropriate indane and Example 771B as the appropriate alcohol, Example 962A was obtained. HRMS calculated for C₄₇H₆₅ClN₂O₆Si: 816.4300; found: 817.4377 (M+H).

### Example 962B methyl (1r,4S,7′S)-4-(3-chloroanilino)-7′-[(2R)-3-{[(5R,8R)-8-hydroxy-5-methyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylate

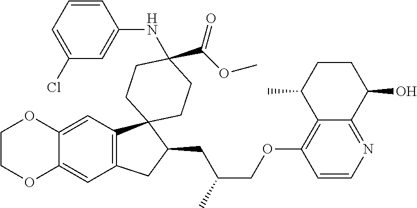 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CC[C@H]2O)OCCO3]

Using General procedure 29 (performing the reaction at 50° C. instead of rt) and Example 962A as the appropriate silyl derivative, Example 962B was obtained. LRMS calculated for C₃₈H₄₅ClN₂O₆: 660.3; found: 661.3 (M+H).

### Example 962 (1r,4S,7′S)-4-(3-chloroanilino)-7′-[(2R)-3-{[(5R,8R)-8-hydroxy-5-methyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylic acid

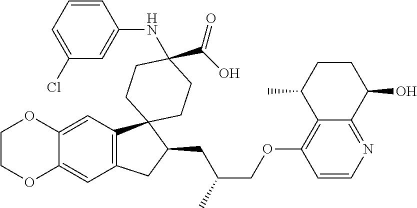 [SMILES: C[C@@H](COc1ccnc2c1[C@H](C)CC[C@H]2O)C[C@H]1Cc2cc3c(cc2[C@]12CC[C@@](Nc1cccc(Cl)c1)(C(=O)O)CC2)OCCO3]

Using General procedure 33a and Example 962B as the appropriate ester, Example 962 was obtained. ¹H NMR (500 MHz, DMSO-d₆) δ ppm: 12.67 (br s, 1H), 8.25 (d, 1H), 7.04 (t, 1H), 6.86 (d, 1H), 6.83 (s, 1H), 6.69 (s, 1H), 6.58 (t, 1H), 6.53 (dm, 1H), 6.52 (dm, 1H), 6.23 (br s, 1H), 4.93 (d, 1H), 4.44 (m, 1H), 4.22-4.13 (m, 4H), 3.93/3.85 (dd+dd, 2H), 3.04 (m, 1H), 2.87/2.41 (dd+dd, 2H), 2.41-1.20 (m, 12H), 2.07 (m, 1H), 1.97 (m, 1H), 1.48/1.32 (m+m, 2H), 1.04 (d, 3H), 1.03 (d, 3H). HRMS calculated for C₃₇H₄₃N₂O₆Cl: 646.2809; found: 647.2885 (M+H).

### Example 963 (1r,4S,7′S)-4-(3-chloroanilino)-7′-[(2R)-3-{[(5R,8R)-5,8-dimethyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylic acid

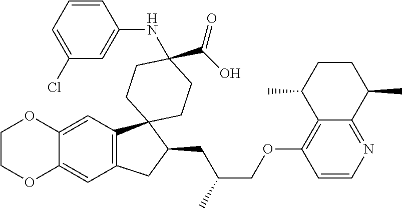 [SMILES: C[C@@H](COc1ccnc2c1[C@H](C)CC[C@H]2C)C[C@H]1Cc2cc3c(cc2[C@]12CC[C@@](Nc1cccc(Cl)c1)(C(=O)O)CC2)OCCO3]

Using General procedure 32 and Example 8380 as the appropriate indane and Example 658G as the appropriate alcohol, Example 963 was obtained. ¹H NMR (500 MHz, DMSO-d₆) δ ppm: 12.65 (br s, 1H), 8.19 (d, 1H), 7.03 (t, 1H), 6.84 (s, 1H), 6.75 (d, 1H), 6.68 (s, 1H), 6.59 (t, 1H), 6.53 (dm, 1H), 6.51 (dm, 1H), 6.25 (br s, 1H), 4.24-4.12 (m, 4H), 3.90/3.83 (dd+dd, 2H), 3.04 (m, 1H), 2.86 (m, 1H), 2.86/2.40 (dd+dd, 2H), 2.45-1.17 (m, 14H), 2.06 (m, 1H), 1.95 (m, 1H), 1.18 (d, 3H), 1.05 (d, 3H), 1.02 (d, 3H). HRMS calculated for C₃₈H₄₅N₂O₅Cl: 644.3017; found: 645.3095 (M+H).

### Example 964 (1r,4S,7′S)-4-(3-chloroanilino)-7′-[(2R)-3-{[(5R,8R)-8-fluoro-5-methyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylic acid

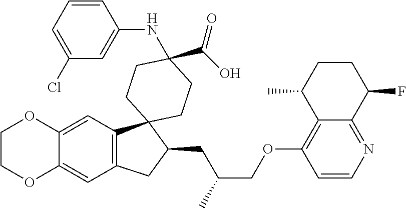 [SMILES: C[C@@H](COc1ccnc2c1[C@H](C)CC[C@H]2F)C[C@H]1Cc2cc3c(cc2[C@]12CC[C@@](Nc1cccc(Cl)c1)(C(=O)O)CC2)OCCO3]

Using General procedure 32 and Example 8380 as the appropriate indane and Example 772B as the appropriate alcohol, Example 964 was obtained. ¹H NMR (500 MHz, DMSO-d₆) δ ppm: 12.55 (br s, 1H), 8.33 (d, 1H), 7.02 (t, 1H), 6.99 (dd, 1H), 6.83 (s, 1H), 6.68 (s, 1H), 6.59 (t, 1H), 6.52 (dd, 1H), 6.51 (dd, 1H), 6.21 (br s, 1H), 5.36 (d, 1H), 4.18 (m, 4H), 3.95/3.90 (dd+dd, 2H), 3.13 (m, 1H), 2.88/2.41 (dd+dd, 2H), 2.39-1.24 (m, 8H), 2.13/2.08 (m+m, 2H), 2.06 (m, 1H), 1.98/1.54 (m+m, 2H), 1.97 (m, 1H), 1.48/1.33 (t+t, 2H), 1.05 (d, 3H), 1.03 (d, 3H). HRMS calculated for C₃₇H₄₂N₂O₅FCl: 648.2766; found: 649.2838 (M+H).

### Example 965 and Example 966

### Example 965A methyl (1r,4S,6′S)-4-(3-chloroanilino)-6′-[(2R)-3-hydroxy-2-methylpropyl]-2′-methyl-6′,7′-dihydro-2′H-spiro[cyclohexane-1,5′-indeno[5,6-d][1,3]dioxole]-4-carboxylate

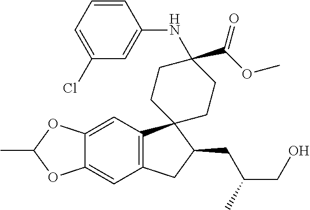 [SMILES: COC(=O)[C@]1(Nc2cccc(Cl)c2)CC[C@@]2(CC1)c1cc3c(cc1C[C@@H]2C[C@@H](C)CO)OC(C)O3]

To a solution of Preparation 20a (400 mg, 0.84 mmol, 1 eq) in DMF (13 mL), were added Cs₂CO₃ (4.12 g, 12.7 mmol, 15 eq) and 1,1-dibromoethane (1.13 mL, 12.7 mmol, 15 eq). The mixture was heated under N₂ at 60° C. for 30 min then cooled to rt. The mixture was filtered, the filtrate was neutralized with 2 M aq. HCl solution, then it was purified via prep RP-HPLC using 25 mM aq. NH₄HCO₃ solution and MeCN as eluents. Example 965A (240 mg, 0.48 mmol, 57%) was obtained as a mixture of diastereoisomers. ¹H NMR (500 MHz, DMSO-d₆) δ ppm: 7.07 (t, 1H), 6.82/6.81 (s/s, 1H), 6.69 (s, 1H), 6.58 (m, 1H), 6.56 (dm, 1H), 6.44 (dm, 1H), 6.28 (s, 1H), 6.27 (m, 1H), 4.44 (br t, 1H), 3.64 (s, 3H), 3.21 (d, 2H), 2.82/2.80 (dd/dd, 1H), 2.44-0.97 (m, 8H), 2.37 (m, 1H), 2.06 (m, 1H), 1.65/1.32 (m+m, 2H), 1.59/1.58 (d/d, 3H), 1.52 (m, 1H), 0.84 (d, 3H). HRMS calculated for C₂₈H₃₄ClNO₅: 499.2126; found: 500.2198 (M+H).

### Example 965B methyl (1r,4S,6′S)-4-(3-chloroanilino)-2′-methyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate, diastereoisomer 1

and

### Example 965C methyl (1r,4S,6′S)-4-(3-chloroanilino)-2′-methyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylate, diastereoisomer 2

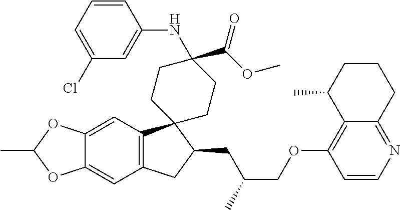 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OC(C)O3]

Using General procedure 30a and Example 965A as the appropriate indane and Preparation 2a1 as the appropriate aryl-alcohol, a mixture of diastereoisomers was obtained. The diastereoisomers were separated by chiral chromatography. Column: AD, 50×500 mm, 20 μm, Eluents: 15:85 EtOH/heptane. The diastereoisomer eluting earlier was collected as Example 965B. ¹H NMR (500 MHz, DMSO-d₆) δ ppm: 8.14 (d, 1H), 7.04 (t, 1H), 6.82 (s, 1H), 6.76 (d, 1H), 6.69 (s, 1H), 6.57 (t, 1H), 6.55 (dd, 1H), 6.44 (dd, 1H), 6.30 (s, 1H), 6.28 (q, 1H), 3.90-3.82 (m, 2H), 3.65 (s, 3H), 3.04 (m, 1H), 2.88/2.41 (dd+dd, 2H), 2.76/2.65 (m+m, 2H), 2.43-1.28 (m, 8H), 2.12 (m, 1H), 1.97 (m, 1H), 1.80/1.74 (m+m, 2H), 1.67/1.61 (m+m, 2H), 1.59 (d, 3H), 1.47/1.30 (t+t, 2H), 1.04 (d, 3H), 1.02 (d, 3H). HRMS calculated for C₃₈H₄₅ClN₂O₅: 644.3017; found 645.3072 (M+H).

The diastereoisomer eluting later was collected as Example 965C. ¹H NMR (500 MHz, DMSO-d₆) δ ppm: 8.14 (d, 1H), 7.04 (t, 1H), 6.83 (s, 1H), 6.76 (d, 1H), 6.70 (s, 1H), 6.57 (t, 1H), 6.55 (dd, 1H), 6.44 (dd, 1H), 6.30 (s, 1H), 6.28 (q, 1H), 3.91-3.81 (m, 2H), 3.65 (s, 3H), 3.05 (m, 1H), 2.88/2.41 (dd+dd, 2H), 2.76/2.65 (m+m, 2H), 2.43-1.28 (m, 8H), 2.12 (m, 1H), 1.97 (m, 1H), 1.80/1.74 (m+m, 2H), 1.67/1.61 (m+m, 2H), 1.58 (d, 3H), 1.47/1.30 (t+t, 2H), 1.05 (d, 3H), 1.03 (d, 3H). HRMS calculated for C₃₈H₄₅ClN₂O₅: 644.3017; found 645.3098 (M+H).

### Example 965 (1r,4S,6′S)-4-(3-chloroanilino)-2′-methyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid, diastereoisomer 1

and

### Example 966 (1r,4S,6′S)-4-(3-chloroanilino)-2′-methyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid, diastereoisomer 2

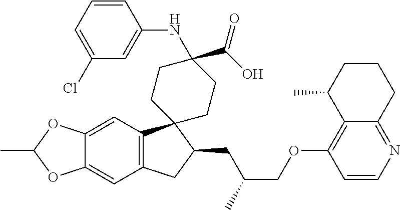 [SMILES: CC1Oc2cc3c(cc2O1)[C@]1(CC[C@@](Nc2cccc(Cl)c2)(C(=O)O)CC1)[C@@H](C[C@@H](C)COc1ccnc2c1[C@H](C)CCC2)C3]

Using General procedure 33a and Example 965B as the appropriate ester, Example 965 was obtained. ¹H NMR (500 MHz, DMSO-d₆) δ ppm: 12.70 (br s, 1H), 8.14 (d, 1H), 7.03 (t, 1H), 6.79 (s, 1H), 6.76 (d, 1H), 6.70 (s, 1H), 6.60 (t, 1H), 6.53 (dd, 1H), 6.52 (dd, 1H), 6.28 (q, 1H), 6.24 (br s, 1H), 3.89/3.85 (dd+dd, 2H), 3.04 (m, 1H), 2.88/2.42 (dd+dd, 2H), 2.76/2.65 (m+m, 2H), 2.41-1.33 (m, 8H), 2.13 (m, 1H), 1.96 (m, 1H), 1.80/1.74 (m+m, 2H), 1.67/1.61 (m+m, 2H), 1.58 (d, 3H), 1.47/1.30 (t+t, 2H), 1.05 (d, 3H), 1.03 (d, 3H). HRMS calculated for C₃₇H₄₃ClN₂O₅: 630.2861; found 631.2937 (M+H).

Using General procedure 33a and Example 965C as the appropriate ester, Example 966 was obtained. ¹H NMR (500 MHz, DMSO-d₆) δ ppm: 12.70 (br s, 1H), 8.14 (d, 1H), 7.03 (t, 1H), 6.80 (s, 1H), 6.77 (d, 1H), 6.70 (s, 1H), 6.60 (t, 1H), 6.53 (dd, 1H), 6.52 (dd, 1H), 6.27 (q, 1H), 6.23 (br s, 1H), 3.90/3.84 (dd+dd, 2H), 3.05 (m, 1H), 2.89/2.41 (dd+dd, 2H), 2.76/2.65 (m+m, 2H), 2.40-1.30 (m, 8H), 2.12 (m, 1H), 1.96 (m, 1H), 1.80/1.74 (m+m, 2H), 1.67/1.61 (m+m, 2H), 1.58 (d, 3H), 1.48/1.31 (t+t, 2H), 1.06 (d, 3H), 1.03 (d, 3H). HRMS calculated for C₃₇H₄₃ClN₂O₅: 630.2861; found 631.2937 (M+H).

### Example 967 (1r,4S,6′S)-4-(3-chloroanilino)-2′-ethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid, diastereoisomer 1

### Example 968 (1r,4S,6′S)-4-(3-chloroanilino)-2′-ethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid, diastereoisomer 2

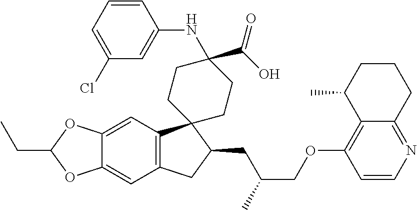 [SMILES: CCC1Oc2cc3c(cc2O1)[C@]1(CC[C@@](Nc2cccc(Cl)c2)(C(=O)O)CC1)[C@@H](C[C@@H](C)COc1ccnc2c1[C@H](C)CCC2)C3]

To a solution of Preparation 20b (200 mg, 0.24 mmol, 1 eq) in DMF (3.6 mL), were added Cs₂CO₃ (1.18 g, 3.63 mmol, 15 eq) and 1,1-dichloropropane (0.39 mL, 3.63 mmol, 15 eq). The mixture was heated under N₂ at 80° C. for 1 h, then cooled to rt. The mixture was filtered, the filtrate was purified via prep RP-HPLC using 25 mM aq. NH₄HCO₃ solution and MeCN and IPA as eluents to obtain a mixture of diastereoisomers. The diastereoisomers were separated by chiral chromatography. Column: AD, 50×500 mm, 20 μm, Eluents: 50:50 EtOH/heptane. The diastereoisomer eluting earlier was hydrolyzed according to General Procedure 33a to obtain Example 967. ¹H NMR (500 MHz, DMSO-d₆) δ ppm: 8.14 (d, 1H), 7.03 (t, 1H), 6.79 (s, 1H), 6.76 (d, 1H), 6.70 (s, 1H), 6.60 (t, 1H), 6.53 (m, 2H), 6.12 (t, 1H), 3.88/3.85 (dd+dd, 2H), 3.03 (m, 1H), 2.88/2.42 (dd+dd, 2H), 2.76/2.65 (m+m, 2H), 2.40-1.34 (m, 8H), 2.13 (m, 1H), 1.96 (m, 1H), 1.91/1.88 (dd+dd, 2H), 1.80/1.73 (m+m, 2H), 1.67/1.61 (m+m, 2H), 1.48/1.29 (t+t, 2H), 1.03 (d, 3H), 1.03 (d, 3H), 0.98 (t, 3H). HRMS calculated for C₃₈H₄₅ClN₂O₅: 644.3017; found: 645.3094 (M+H).

The diastereoisomer eluting later was hydrolyzed according to General Procedure 33a to obtain Example 968. ¹H NMR (500 MHz, DMSO-d₆) δ ppm: 12.76 (br s, 1H), 8.14 (d, 1H), 7.00 (t, 1H), 6.81 (s, 1H), 6.76 (d, 1H), 6.69 (s, 1H), 6.61 (t, 1H), 6.53 (dd, 1H), 6.48 (d, 1H), 6.14 (br s, 1H), 6.12 (t, 1H), 3.90/3.84 (dd+dd, 2H), 3.05 (m, 1H), 2.88/2.40 (dd+dd, 2H), 2.76/2.65 (m+m, 2H), 2.42-1.26 (m, 14H), 2.13 (m, 1H), 1.96 (m, 1H), 1.88 (m, 2H), 1.06 (d, 3H), 1.03 (d, 3H), 0.97 (t, 3H). HRMS calculated for C₃₈H₄₅ClN₂O₅: 644.3017; found: 645.3091 (M+H).

### Example 971

### Example 971A (2R)-propane-1,2-diyl bis(4-methylbenzene-1-sulfonate)

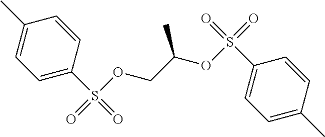

Using General procedure 42 and (2R)-propane-1,2-diol as the appropriate alcohol, Example 971A was obtained. ¹H NMR (400 MHz, DMSO-d₆) δ ppm: 7.71 (d, 2H), 7.70 (d, 2H), 7.47 (d, 2H), 7.43 (d, 2H), 4.71 (m, 1H), 4.01 (d, 2H), 2.43 (s, 3H), 2.41 (s, 3H), 1.14 (d, 3H). HRMS calculated for C₁₇H₂₀O6S₂: 384.0701; found 402.1032 (M+NH₄).

### Example 971B methyl (1r,2′S,4S,7′S)-4-(3-chloroanilino)-7′-[(2R)-3-hydroxy-2-methylpropyl]-2′-methyl-2′,3′,7′,8′-tetrahydrospiro[cyclohexane-1,6′-indeno[5,6-b][1,4]dioxine]-4-carboxylate and methyl (1r,3'S,4S,7′S)-4-(3-chloroanilino)-7′-[(2R)-3-hydroxy-2-methylpropyl]-3′-methyl-2′,3′,7′,8′-tetrahydrospiro[cyclohexane-1,6′-indeno[5,6-b][1,4]dioxine]-4-carboxylate

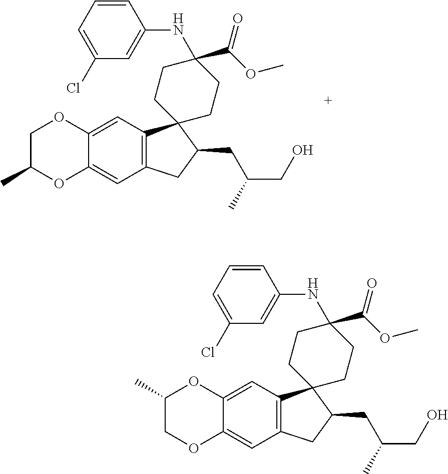 [SMILES: COC(=O)[C@]1(Nc2cccc(Cl)c2)CC[C@@]2(CC1)c1cc3c(cc1C[C@@H]2C[C@@H](C)CO)O[C@@H](C)CO3 ; COC(=O)[C@]1(Nc2cccc(Cl)c2)CC[C@@]2(CC1)c1cc3c(cc1C[C@@H]2C[C@@H](C)CO)OC[C@H](C)O3]

Using General procedure 43 and Preparation 20a as the appropriate catechol and Example 971A as the appropriate bis-tosylate, Example 971B was obtained as a mixture of regioisomers. ¹H NMR (400 MHz, DMSO-d₆) δ ppm: 7.07 (t, 1H), 6.84/6.83 (s/s, 1H), 6.69/6.67 (s/s, 1H), 6.60-6.53 (m, 2H), 6.44 (dd, 1H), 6.30 (s, 1H), 4.45 (br t, 1H), 4.23 (m, 2H), 3.75 (m, 1H), 3.65 (s, 3H), 3.22 (br d, 2H), 2.82/2.80 (dd/dd, 1H), 2.37 (m, 1H), 2.36-1.74 (m, 6H), 1.67 (t, 1H), 1.59-1.31 (m, 3H), 1.27 (dd, 4H), 1.04 (t, 1H), 0.84 (d, 3H). HRMS calculated for C₂₉H₃₆ClNO₅: 513.2282; found: 514.2361 (M+H).

### Example 971C methyl (1r,2′S,4S,7′S)-4-(3-chloroanilino)-2′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

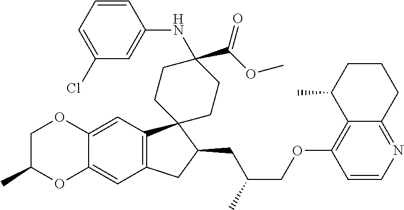 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)O[C@@H](C)CO3]

and

### Example 971D methyl (1r,3'S,4S,7′S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

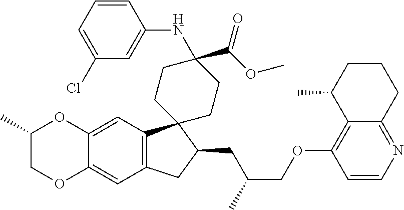 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OC[C@H](C)O3]

Using General procedure 30a and Example 971B as the appropriate indane and Preparation 2a1 as the appropriate aryl-alcohol, a mixture of regioisomers was obtained. The regioisomers were separated by chiral chromatography. Column: AD, 50×500 mm, 20 μm, Eluents: 15:85 EtOH/heptane. The regioisomer eluting earlier was collected as Example 971C. ¹H NMR (500 MHz, DMSO-d₆) δ ppm: 8.14 (d, 1H), 7.04 (t, 1H), 6.85 (s, 1H), 6.77 (d, 1H), 6.70 (s, 1H), 6.57 (t, 1H), 6.55 (dd, 1H), 6.42 (dd, 1H), 6.31 (s, 1H), 4.24/3.76 (dd+dd, 2H), 4.20 (m, 1H), 3.90/3.84 (dd+dd, 2H), 3.64 (s, 3H), 3.05 (m, 1H), 2.87/2.39 (dd+dd, 2H), 2.76/2.66 (m+m, 2H), 2.40-1.23 (m, 8H), 2.06 (m, 1H), 1.96 (m, 1H), 1.80/1.74 (m+m, 2H), 1.68/1.62 (m+m, 2H), 1.48/1.31 (t+t, 2H), 1.26 (d, 3H), 1.06 (d, 3H), 1.02 (d, 3H). HRMS calculated for C₃₉H₄₇ClN₂O₅: 658.3174; found 659.3252 (M+H).

The regioisomer eluting later was collected as Example 971D. ¹H NMR (500 MHz, DMSO-d₆) δ ppm: 8.14 (d, 1H), 7.04 (t, 1H), 6.83 (s, 1H), 6.77 (d, 1H), 6.69 (s, 1H), 6.57 (t, 1H), 6.55 (dd, 1H), 6.42 (dd, 1H), 6.31 (s, 1H), 4.24/3.73 (dd+dd, 2H), 4.22 (m, 1H), 3.90/3.84 (dd+dd, 2H), 3.64 (s, 3H), 3.05 (m, 1H), 2.87/2.40 (dd+dd, 2H), 2.76/2.66 (m+m, 2H), 2.40-1.22 (m, 8H), 2.06 (m, 1H), 1.96 (m, 1H), 1.81/1.74 (m+m, 2H), 1.68/1.62 (m+m, 2H), 1.49/1.31 (t+t, 2H), 1.27 (d, 3H), 1.06 (d, 3H), 1.02 (d, 3H). HRMS calculated for C₃₉H₄₇ClN₂O₅: 658.3174; found 659.3253 (M+H).

### Example 971 (1r,2′S,4S,7′S)-4-(3-chloroanilino)-2′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

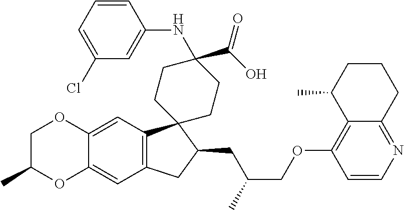 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OC[C@H](C)O3]

Using General procedure 33a and Example 971C as the appropriate ester, Example 971 was obtained. ¹H NMR (500 MHz, DMSO-d₆) δ ppm: 12.70 (br s, 1H), 8.14 (d, 1H), 7.03 (t, 1H), 6.84 (s, 1H), 6.77 (d, 1H), 6.67 (s, 1H), 6.59 (t, 1H), 6.53 (dd, 1H), 6.51 (dd, 1H), 6.22 (br s, 1H), 4.23/3.75 (dd+dd, 2H), 4.19 (m, 1H), 3.90/3.84 (dd+dd, 2H), 3.06 (m, 1H), 2.87/2.40 (dd+dd, 2H), 2.76/2.66 (m+m, 2H), 2.38-1.23 (m, 8H), 2.06 (m, 1H), 1.96 (m, 1H), 1.74/1.80 (m+m, 2H), 1.67/1.62 (m+m, 2H), 1.48/1.32 (t+t, 2H), 1.26 (d, 3H), 1.07 (d, 3H), 1.02 (d, 3H). HRMS calculated for C₃₈H₄₅ClN₂O₅: 644.3017; found 645.3088 (M+H).

### Example 972 (1r,3'S,4S,7′S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

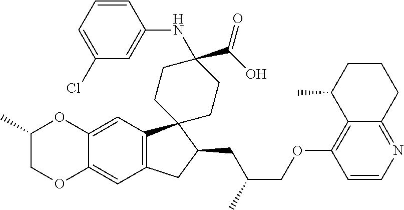 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)O[C@@H](C)CO3]

Using General procedure 33a and Example 971D as the appropriate ester, Example 972 was obtained. ¹H NMR (500 MHz, DMSO-d₆) δ ppm: 12.69 (br s, 1H), 8.14 (d, 1H), 7.03 (t, 1H), 6.83 (s, 1H), 6.77 (d, 1H), 6.69 (s, 1H), 6.59 (t, 1H), 6.53 (dd, 1H), 6.51 (dd, 1H), 6.22 (br s, 1H), 4.24/3.73 (m+dd, 2H), 4.22 (m, 1H), 3.91/3.84 (dd+dd, 2H), 3.06 (m, 1H), 2.87/2.40 (dd+dd, 2H), 2.76/2.65 (br d+m, 2H), 2.40-1.19 (m, 14H), 2.05 (m, 1H), 1.96 (m, 1H), 1.27 (d, 3H), 1.07 (d, 3H), 1.02 (d, 3H). HRMS calculated for C₃₈H₄₅ClN₂O₅: 644.3017; found 645.3092 (M+H).

### Example 973

### Example 973A (2S)-propane-1,2-diyl bis(4-methylbenzene-1-sulfonate)

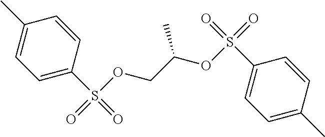

Using General procedure 42 and (2S)-propane-1,2-diol as the appropriate alcohol, Example 973A was obtained. ¹H NMR (400 MHz, DMSO-d₆) δ ppm: 7.71 (d, 2H), 7.70 (d, 2H), 7.47 (d, 2H), 7.43 (d, 2H), 4.71 (m, 1H), 4.01 (d, 2H), 2.43 (s, 3H), 2.41 (s, 3H), 1.14 (d, 3H). HRMS calculated for C₁₇H₂₀O6S₂: 384.0701; found 402.1032 (M+NH₄).

### Example 973B methyl (1r,3′R,4S,7′S)-4-(3-chloroanilino)-7′-[(2R)-3-hydroxy-2-methylpropyl]-3′-methyl-2′,3′,7′,8′-tetrahydrospiro[cyclohexane-1,6′-indeno[5,6-b][1,4]dioxine]-4-carboxylate and methyl (1r,2′R,4S,7′S)-4-(3-chloroanilino)-7′-[(2R)-3-hydroxy-2-methylpropyl]-2′-methyl-2′,3′,7′,8′-tetrahydrospiro[cyclohexane-1,6′-indeno[5,6-b][1,4]dioxine]-4-carboxylate

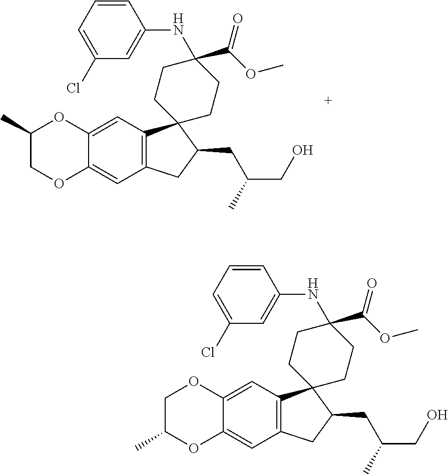 [SMILES: COC(=O)[C@]1(Nc2cccc(Cl)c2)CC[C@@]2(CC1)c1cc3c(cc1C[C@@H]2C[C@@H](C)CO)OC[C@@H](C)O3 ; COC(=O)[C@]1(Nc2cccc(Cl)c2)CC[C@@]2(CC1)c1cc3c(cc1C[C@@H]2C[C@@H](C)CO)O[C@H](C)CO3]

Using General procedure 43 and Preparation 20a as the appropriate catechol and Example 973A as the appropriate bis-tosylate, Example 973B was obtained as a mixture of regioisomers. ¹H NMR (400 MHz, DMSO-d₆) δ ppm: 7.07 (t, 1H), 6.84/6.83 (s/s, 1H), 6.69/6.67 (s/s, 1H), 6.60-6.53 (m, 2H), 6.44 (dd, 1H), 6.30 (s, 1H), 4.45 (br t, 1H), 4.23 (m, 2H), 3.75 (m, 1H), 3.65 (s, 3H), 3.22 (br d, 2H), 2.82/2.80 (dd/dd, 1H), 2.37 (m, 1H), 2.36-1.74 (m, 6H), 1.67 (t, 1H), 1.59-1.31 (m, 3H), 1.27 (dd, 4H), 1.04 (t, 1H), 0.84 (d, 3H). HRMS calculated for C₂₉H₃₆ClNO₅: 513.2282; found: 514.2361 (M+H).

### Example 973C methyl (1r,3′R,4S,7′S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

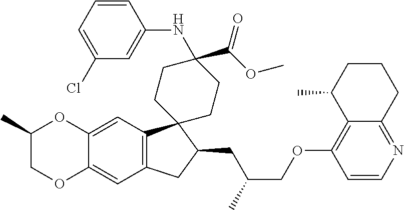 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OC[C@@H](C)O3]

and

### Example 973D methyl (1r,2′R,4S,7′S)-4-(3-chloroanilino)-2′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

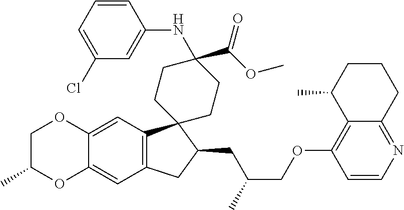 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)O[C@H](C)CO3]

Using General procedure 30a and Example 973B as the appropriate indane and Preparation 2a1 as the appropriate aryl-alcohol, a mixture of regioisomers was obtained. The regioisomers were separated by chiral chromatography. Column: AD, 50×500 mm, 20 μm, Eluents: 15:85 EtOH/heptane. The regioisomer eluting earlier was collected as Example 973C. ¹H NMR (500 MHz, DMSO-d₆) δ ppm: 8.14 (d, 1H), 7.04 (t, 1H), 6.83 (s, 1H), 6.77 (d, 1H), 6.69 (s, 1H), 6.57 (t, 1H), 6.55 (dd, 1H), 6.42 (dd, 1H), 6.31 (s, 1H), 4.24/3.74 (dd+dd, 2H), 4.18 (td, 1H), 3.90/3.85 (dd+dd, 2H), 3.64 (s, 3H), 3.05 (m, 1H), 2.86/2.40 (dd+dd, 2H), 2.76/2.66 (m+m, 2H), 2.41-1.22 (m, 8H), 2.06 (m, 1H), 1.97 (m, 1H), 1.81/1.74 (m+m, 2H), 1.68/1.62 (m+m, 2H), 1.48/1.31 (t+t, 2H), 1.28 (d, 3H), 1.06 (d, 3H), 1.02 (d, 3H). HRMS calculated for C₃₉H₄₇ClN₂O₅: 658.3174; found 659.3248 (M+H).

The regioisomer eluting later was collected as Example 973D. ¹H NMR (500 MHz, DMSO-d₆) δ ppm: 8.14 (d, 1H), 7.04 (t, 1H), 6.85 (s, 1H), 6.77 (d, 1H), 6.67 (s, 1H), 6.57 (t, 1H), 6.55 (dd, 1H), 6.42 (dd, 1H), 6.30 (s, 1H), 4.24/3.74 (dd+dd, 2H), 4.21 (m, 1H), 3.90/3.84 (dd+dd, 2H), 3.64 (s, 3H), 3.05 (m, 1H), 2.86/2.40 (dd+dd, 2H), 2.76/2.65 (m+m, 2H), 2.40-1.22 (m, 8H), 2.06 (m, 1H), 1.96 (m, 1H), 1.80/1.72 (m+m, 2H), 1.68/1.62 (m+m, 2H), 1.48/1.31 (t+t, 2H), 1.26 (d, 3H), 1.06 (d, 3H), 1.02 (d, 3H). HRMS calculated for C₃₉H₄₇ClN₂O₅: 658.3174; found 659.3251 (M+H).

### Example 973 (1r,3′R,4S,7′S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

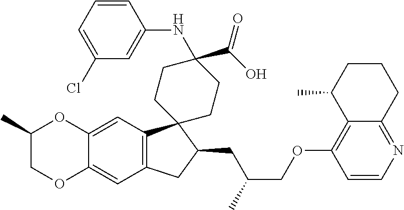 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)O[C@H](C)CO3]

Using General procedure 33a and Example 973C as the appropriate ester, Example 973 was obtained. ¹H NMR (500 MHz, DMSO-d₆) δ ppm: 12.68 (br s, 1H), 8.14 (d, 1H), 7.03 (t, 1H), 6.83 (s, 1H), 6.77 (d, 1H), 6.69 (s, 1H), 6.59 (t, 1H), 6.53 (dd, 1H), 6.51 (dd, 1H), 6.24 (br s, 1H), 4.24/3.74 (dd+dd, 2H), 4.19 (m, 1H), 3.90/3.85 (dd+dd, 2H), 3.06 (m, 1H), 2.86/2.40 (dd+dd, 2H), 2.76/2.65 (m+m, 2H), 2.38-1.22 (m, 8H), 2.06 (m, 1H), 1.96 (m, 1H), 1.80/1.74 (m+m, 2H), 1.68/1.62 (m+m, 2H), 1.49/1.32 (t+t, 2H), 1.28 (d, 3H), 1.06 (d, 3H), 1.02 (d, 3H). HRMS calculated for C₃₈H₄₅ClN₂O₅: 644.3017; found 645.3086 (M+H).

### Example 974 (1r,2′R,4S,7′S)-4-(3-chloroanilino)-2′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

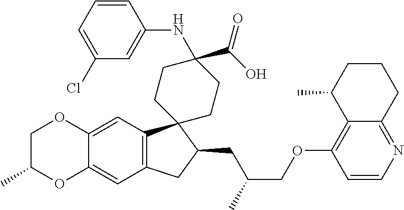 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OC[C@@H](C)O3]

Using General procedure 33a and Example 973D as the appropriate ester, Example 974 was obtained. ¹H NMR (500 MHz, DMSO-d₆) δ ppm: 12.66 (br s, 1H), 8.14 (d, 1H), 7.04 (t, 1H), 6.85 (s, 1H), 6.77 (d, 1H), 6.67 (s, 1H), 6.59 (t, 1H), 6.53 (dd, 1H), 6.51 (dd, 1H), 6.22 (br s, 1H), 4.23/3.74 (dd+dd, 2H), 4.20 (m, 1H), 3.90/3.84 (dd+dd, 2H), 3.06 (m, 1H), 2.86/2.40 (dd+dd, 2H), 2.76/2.65 (m+m, 2H), 2.39-1.22 (m, 8H), 2.06 (m, 1H), 1.96 (m, 1H), 1.81/1.74 (m+m, 2H), 1.67/1.62 (m+m, 2H), 1.48/1.31 (t+t, 2H), 1.26 (d, 3H), 1.07 (d, 3H), 1.02 (d, 3H). HRMS calculated for C₃₈H₄₅ClN₂O₅: 644.3017; found 645.3092 (M+H).

### Example 975

### Example 975A (2R,3R)-butane-2,3-diyl bis(4-methylbenzene-1-sulfonate)

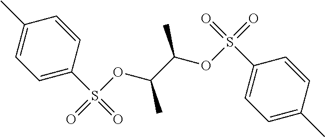

Using General procedure 42 and (2R,3R)-butane-2,3-diol as the appropriate alcohol, Example 975A was obtained. ¹H NMR (500 MHz, DMSO-d₆) δ ppm: 7.74 (m, 4H), 7.46 (m, 4H), 4.57 (m, 2H), 2.42 (s, 6H), 1.01 (d, 6H). HRMS calculated for C₁₈H₂₂O₆S₂: 398.0858; found 399.0931 (M+H).

### Example 975B methyl (1r,2′S,3'S,4S,7′S)-4-(3-chloroanilino)-2′,3′-dimethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

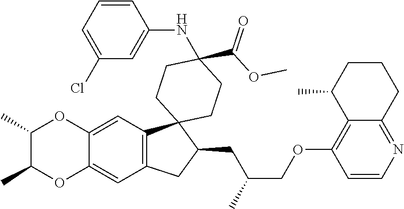 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)O[C@@H](C)[C@H](C)O3]

Using General procedure 43 and Preparation 20b as the appropriate catechol and Example 975A as the appropriate bis-tosylate, Example 975B was obtained. ¹H NMR (500 MHz, DMSO-d₆) δ ppm: 8.14 (d, 1H), 7.04 (t, 1H), 6.83 (s, 1H), 6.77 (d, 1H), 6.67 (s, 1H), 6.57 (t, 1H), 6.55 (dd, 1H), 6.42 (dd, 1H), 6.31 (s, 1H), 3.93-3.82 (m, 2H), 3.82 (m, 2H), 3.64 (s, 3H), 3.05 (m, 1H), 2.87/2.39 (dd+dd, 2H), 2.76/2.65 (br d+m, 2H), 2.44-1.20 (m, 14H), 2.05 (m, 1H), 1.96 (m, 1H), 1.28/1.27 (d+d, 6H), 1.07 (d, 3H), 1.02 (d, 3H). HRMS calculated for C₄₀H₄₉ClN₂O₅: 672.3333; found 673.3408 (M+H).

### Example 975 (1r,2′S,3'S,4S,7′S)-4-(3-chloroanilino)-2′,3′-dimethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

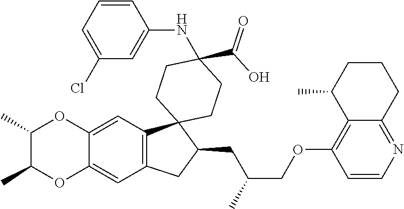 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)O[C@@H](C)[C@H](C)O3]

Using General procedure 33a and Example 975B as the appropriate ester, Example 975 was obtained. ¹H NMR (500 MHz, DMSO-d₆) δ ppm: 12.67 (br s, 1H), 8.14 (d, 1H), 7.03 (t, 1H), 6.83 (s, 1H), 6.77 (d, 1H), 6.67 (s, 1H), 6.59 (t, 1H), 6.53 (dm, 1H), 6.51 (dm, 1H), 6.22 (br s, 1H), 3.93-3.82 (m, 2H), 3.82 (m, 2H), 3.05 (m, 1H), 2.87/2.39 (dd+dd, 2H), 2.76/2.65 (br d+m, 2H), 2.40-1.19 (m, 14H), 2.05 (m, 1H), 1.96 (m, 1H), 1.28/1.27 (d+d, 6H), 1.07 (d, 3H), 1.02 (d, 3H). HRMS calculated for C₃₉H₄₇ClN₂O₅: 658.3173; found 659.3246 (M+H).

### Example 976

### Example 976A (2S,3S)-butane-2,3-diyl bis(4-methylbenzene-1-sulfonate)

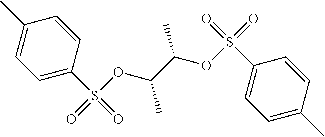

Using General procedure 42 and (2S,3S)-butane-2,3-diol as the appropriate alcohol, Example 976A was obtained. ¹H NMR (500 MHz, DMSO-d₆) δ ppm: 7.74 (m, 4H), 7.46 (m, 4H), 4.57 (m, 2H), 2.42 (s, 6H), 1.01 (d, 6H). HRMS calculated for C₁₈H₂₂O₆S₂: 398.0858; found 421.0753 (M+Na).

### Example 976B methyl (1r,2′R,3′R,4S,7′S)-4-(3-chloroanilino)-2′,3′-dimethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylate

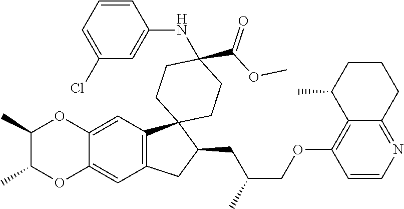 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)O[C@H](C)[C@@H](C)O3]

Using General procedure 43 and Preparation 20b as the appropriate catechol and Example 976A as the appropriate bis-tosylate, Example 976B was obtained. ¹H NMR (500 MHz, DMSO-d₆) δ ppm: 8.14 (d, 1H), 7.04 (t, 1H), 6.84 (s, 1H), 6.77 (d, 1H), 6.67 (s, 1H), 6.57 (t, 1H), 6.55 (dd, 1H), 6.42 (dd, 1H), 6.31 (s, 1H), 3.90/3.85 (dd+dd, 2H), 3.82 (m, 1H), 3.82 (m, 1H), 3.64 (s, 3H), 3.05 (m, 1H), 2.86/2.40 (dd+dd, 2H), 2.76/2.66 (m+m, 2H), 2.41-1.21 (m, 8H), 2.06 (m, 1H), 1.96 (m, 1H), 1.80/1.74 (m+m, 2H), 1.68/1.62 (m+m, 2H), 1.48/1.31 (t+t, 2H), 1.29 (d, 3H), 1.27 (d, 3H), 1.06 (d, 3H), 1.02 (d, 3H). HRMS calculated for C₄₀H₄₉ClN₂O₅: 672.3333; found 673.3404 (M+H).

### Example 976 (1r,2′R,3′R,4S,7′S)-4-(3-chloroanilino)-2′,3′-dimethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

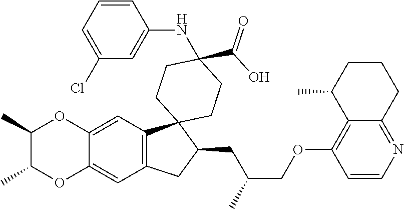 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)O[C@H](C)[C@@H](C)O3]

Using General procedure 33a and Example 976B as the appropriate ester, Example 976 was obtained. ¹H NMR (500 MHz, DMSO-d₆) δ ppm: 12.66 (br s, 1H), 8.14 (d, 1H), 7.03 (t, 1H), 6.84 (s, 1H), 6.77 (d, 1H), 6.67 (s, 1H), 6.59 (t, 1H), 6.52 (dd, 1H), 6.51 (dd, 1H), 6.21 (br s, 1H), 3.90/3.84 (dd+dd, 2H), 3.82 (m, 2H), 3.06 (m, 1H), 2.86/2.40 (dd+dd, 2H), 2.76/2.65 (br d+m, 2H), 2.39-1.19 (m, 14H), 2.05 (br s, 1H), 1.96 (m, 1H), 1.28/1.26 (d+d, 6H), 1.07 (d, 3H), 1.02 (d, 3H). HRMS calculated for C₃₉H₄₇ClN₂O₅: 658.3173; found 659.3247 (M+H).

### Example 977 and Example 978

### Example 977A (2R)-butane-1,2-diyl bis(4-methylbenzene-1-sulfonate)

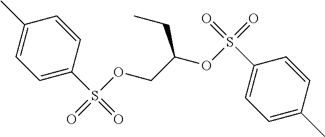

Using General procedure 42 and (2R)-butane-1,2-diol as the appropriate alcohol, Example 977A was obtained. ¹H NMR (400 MHz, DMSO-d₆) δ ppm: 7.71/7.69 (d+d, 4H), 7.47/7.43 (d+d, 4H), 4.59 (m, 1H), 4.04/4.00 (dd+dd, 2H), 2.43/2.41 (s+s, 6H), 1.58-1.48 (m, 2H), 0.67 (t, 3H). HRMS calculated for C₁₈H₂₂O₆S₂: 398.0858; found 421.0748 (M+Na).

### Example 977 (1r,3'S,4S,7′S)-4-(3-chloroanilino)-3′-ethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

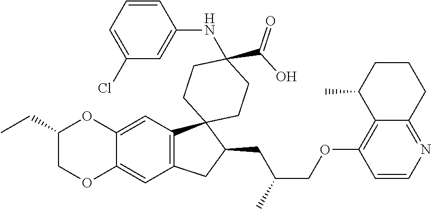 [SMILES: CC[C@H]1COc2cc3c(cc2O1)[C@]1(CC[C@@](Nc2cccc(Cl)c2)(C(=O)O)CC1)[C@@H](C[C@@H](C)COc1ccnc2c1[C@H](C)CCC2)C3]

and

### Example 978 (1r,2′S,4S,7′S)-4-(3-chloroanilino)-2′-ethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

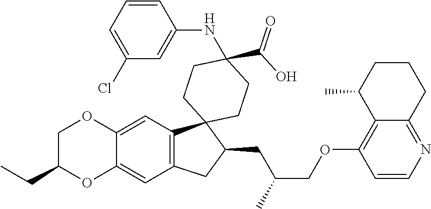 [SMILES: CC[C@H]1COc2cc3c(cc2O1)C[C@H](C[C@@H](C)COc1ccnc2c1[C@H](C)CCC2)[C@]31CC[C@](Nc2cccc(Cl)c2)(C(=O)O)CC1]

Using General procedure 43 and Preparation 20a as the appropriate catechol and Example 977A as the appropriate bis-tosylate, a mixture of regioisomers was obtained. The regioisomers were separated by chiral chromatography. Column: (R, R) WHELK-02, 50×500 mm, 10 μm, Eluents: 15:85 EtOH/heptane. The regioisomer eluting earlier was collected and hydrolyzed according to General procedure 33a to obtain Example 977. ¹H NMR (500 MHz, DMSO-d₆) δ ppm: 12.70 (br s, 1H), 8.14 (d, 1H), 7.02 (t, 1H), 6.82 (s, 1H), 6.76 (d, 1H), 6.68 (s, 1H), 6.60 (t, 1H), 6.52 (dd, 1H), 6.52 (dd, 1H), 6.20 (br s, 1H), 4.25/3.80 (dd+dd, 2H), 4.01 (m, 1H), 3.90/3.84 (dd+dd, 2H), 3.06 (m, 1H), 2.87/2.40 (dd+dd, 2H), 2.77/2.66 (m+m, 2H), 2.38-1.26 (m, 8H), 2.07 (m, 1H), 1.97 (m, 1H), 1.80/1.74 (m+m, 2H), 1.68/1.62 (m+m, 2H), 1.62 (m, 2H), 1.48/1.32 (t+t, 2H), 1.07 (d, 3H), 1.03 (d, 3H), 0.99 (t, 3H). HRMS calculated for C₃₉H₄₇ClN₂O₅: 658.3173; found 659.3250 (M+H).

The regioisomer eluting later was collected and hydrolyzed according to General procedure 33a to obtain Example 978. ¹H NMR (500 MHz, DMSO-d₆) δ ppm: 12.69 (br s, 1H), 8.14 (d, 1H), 7.03 (t, 1H), 6.83 (s, 1H), 6.77 (d, 1H), 6.67 (s, 1H), 6.59 (t, 1H), 6.53 (dd, 1H), 6.51 (dd, 1H), 6.19 (br s, 1H), 4.24/3.83 (dd+dd, 2H), 4.00 (dd, 1H), 3.90/3.84 (dd+dd, 2H), 3.05 (m, 1H), 2.87/2.40 (dd+dd, 2H), 2.76/2.65 (m+m, 2H), 2.39-1.25 (m, 8H), 2.07 (m, 1H), 1.97 (m, 1H), 1.81/1.74 (m+m, 2H), 1.69/1.63 (m+m, 2H), 1.61 (m, 2H), 1.48/1.31 (t+t, 2H), 1.06 (d, 3H), 1.03 (d, 3H), 0.99 (t, 3H). HRMS calculated for C₃₉H₄₇ClN₂O₅: 658.3173; found 659.3248 (M+H).

### Example 979 and Example 980

### Example 979A (2S)-butane-1,2-diyl bis(4-methylbenzene-1-sulfonate)

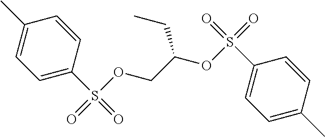

Using General procedure 42 and (2S)-butane-1,2-diol as the appropriate alcohol, Example 979A was obtained. HRMS calculated for C₁₈H₂₂O₆S₂: 398.0858; found 421.0748 (M+Na).

### Example 979 (1r,3′R,4S,7′S)-4-(3-chloroanilino)-3′-ethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

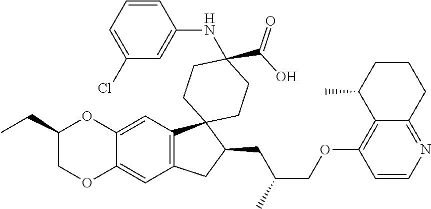 [SMILES: CC[C@@H]1COc2cc3c(cc2O1)[C@]1(CC[C@@](Nc2cccc(Cl)c2)(C(=O)O)CC1)[C@@H](C[C@@H](C)COc1ccnc2c1[C@H](C)CCC2)C3]

and

### Example 980 (1r,2′R,4S,7′S)-4-(3-chloroanilino)-2′-ethyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid

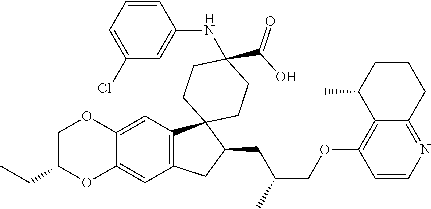 [SMILES: CC[C@@H]1COc2cc3c(cc2O1)C[C@H](C[C@@H](C)COc1ccnc2c1[C@H](C)CCC2)[C@]31CC[C@](Nc2cccc(Cl)c2)(C(=O)O)CC1]

Using General procedure 43 and Preparation 20a as the appropriate catechol and Example 979A as the appropriate bis-tosylate, a mixture of regioisomers was obtained. The regioisomers were separated by chiral chromatography. Column: (R, R) WHELK-02, 50×500 mm, 10 μm, Eluents: 15:85 EtOH/heptane. The regioisomer eluting earlier was collected and hydrolyzed according to General procedure 33a to obtain Example 979. ¹H NMR (500 MHz, DMSO-d₆) δ ppm: 12.69 (br s, 1H), 8.14 (d, 1H), 7.03 (t, 1H), 6.82 (s, 1H), 6.77 (d, 1H), 6.68 (s, 1H), 6.60 (t, 1H), 6.53 (dd, 1H), 6.52 (dd, 1H), 6.21 (br s, 1H), 4.26/3.79 (dd+dd, 2H), 3.97 (dd, 1H), 3.90/3.84 (dd+dd, 2H), 3.05 (m, 1H), 2.86/2.40 (dd+dd, 2H), 2.76/2.65 (m+m, 2H), 2.39-1.24 (m, 8H), 2.07 (m, 1H), 1.97 (m, 1H), 1.80/1.74 (m+m, 2H), 1.68/1.62 (m+m, 2H), 1.62 (m, 2H), 1.48/1.31 (t+t, 2H), 1.06 (d, 3H), 1.03 (d, 3H), 1.00 (t, 3H). HRMS calculated for C₃₉H₄₇ClN₂O₅: 658.3173; found 659.3248 (M+H).

The regioisomer eluting later was collected and hydrolyzed according to General procedure 33a to obtain Example 980. ¹H NMR (500 MHz, DMSO-d₆) δ ppm: 12.69 (br s, 1H), 8.14 (d, 1H), 7.03 (t, 1H), 6.83 (s, 1H), 6.77 (d, 1H), 6.67 (s, 1H), 6.59 (t, 1H), 6.53 (dd, 1H), 6.51 (dd, 1H), 6.20 (br s, 1H), 4.24/3.82 (dd+dd, 2H), 4.00 (dd, 1H), 3.90/3.85 (dd+dd, 2H), 3.05 (m, 1H), 2.86/2.40 (dd+dd, 2H), 2.76/2.65 (m+m, 2H), 2.40-1.24 (m, 8H), 2.07 (m, 1H), 1.96 (m, 1H), 1.81/1.74 (m+m, 2H), 1.68/1.62 (m+m, 2H), 1.60 (m, 2H), 1.48/1.31 (t+t, 2H), 1.06 (d, 3H), 1.02 (d, 3H), 0.99 (t, 3H). HRMS calculated for C₃₉H₄₇ClN₂O₅: 658.3173; found 659.3249 (M+H).

### Example 981

### Example 981A 2,2-dimethylpropane-1,3-diyl bis(4-methylbenzene-1-sulfonate)

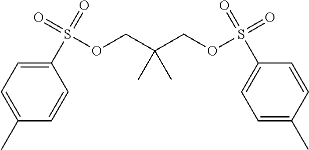 [SMILES: Cc1ccc(S(=O)(=O)OCC(C)(C)COS(=O)(=O)c2ccc(C)cc2)cc1]

Using General procedure 42 and 2,2-dimethylpropane-1,3-diol as the appropriate alcohol, Example 981A was obtained. ¹H NMR (500 MHz, DMSO-d₆) δ ppm: 7.74 (dm, 4H), 7.48 (dm, 4H), 3.72 (s, 4H), 2.43 (s, 6H), 0.78 (s, 6H). HRMS calculated for C₁₉H₂₄O₆S₂: 412.1014; found 413.1089 (M+H).

### Example 981B methyl (1r,4S,8′S)-4-(3-chloroanilino)-3′,3′-dimethyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate

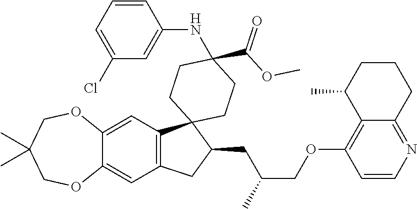 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCC(C)(C)CO3]

Using General procedure 43 and Preparation 20b as the appropriate catechol and Example 981A as the appropriate bis-tosylate Example 981B was obtained. The crude product was purified via prep RP-HPLC using 0.1 V/V % aq. TFA solution and MeCN as eluents to afford Example 981B as a TFA salt. ¹H NMR (500 MHz, DMSO-d₆) δ ppm: 14.62 (br s, 1H), 8.60 (d, 1H), 7.44 (d, 1H), 7.05 (t, 1H), 6.93 (s, 1H), 6.78 (s, 1H), 6.58 (t, 1H), 6.56 (d, 1H), 6.42 (d, 1H), 6.31 (br s, 1H), 4.23/4.16 (dd+dd, 2H), 3.80-3.68 (m, 4H), 3.64 (s, 3H), 3.10 (m, 1H), 3.00-2.82 (m, 2H), 2.89/2.42 (dd+dd, 2H), 2.43-1.22 (m, 14H), 2.11 (br s, 1H), 2.04 (m, 1H), 1.09 (d, 3H), 1.05 (d, 3H), 0.99/0.95 (s+s, 6H). HRMS calculated for C₄₁H₅₁ClN₂O₅: 686.3486; found 687.3565 (M+H).

### Example 981 (1r,4S,8′S)-4-(3-chloroanilino)-3′,3′-dimethyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

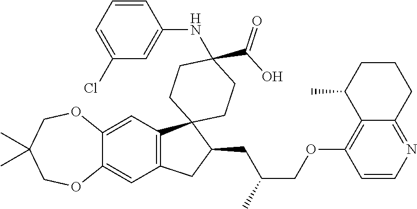 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC(C)(C)CO3]

Using General procedure 33a and Example 981B as the appropriate ester, Example 981 was obtained. ¹H NMR (500 MHz, DMSO-d₆) δ ppm: 12.72 (br s, 1H), 8.14 (d, 1H), 7.03 (t, 1H), 6.93 (s, 1H), 6.77 (t, 2H), 6.59 (t, 1H), 6.53 (dd, 1H), 6.51 (dd, 1H), 6.22 (br s, 1H), 3.90/3.84 (dd+dd, 2H), 3.76/3.72 (dd+dd, 4H), 3.05 (m, 1H), 2.88/2.41 (dd+dd, 2H), 2.76/2.65 (m+m, 2H), 2.39-1.25 (m, 8H), 2.08 (m, 1H), 1.97 (m, 1H), 1.81/1.74 (m+m, 2H), 1.68/1.62 (m+m, 2H), 1.48/1.31 (t+t, 2H), 1.06 (d, 3H), 1.02 (d, 3H), 0.98/0.95 (s+s, 6H). HRMS calculated for C₄₀H₄₉ClN₂O₅: 672.3330; found 673.3407 (M+H).

### Example 982 and Example 983

### Example 982A 2-methylpropane-1,3-diyl bis(4-methylbenzene-1-sulfonate)

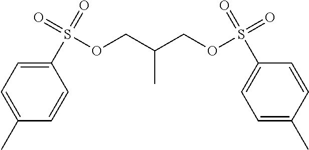 [SMILES: Cc1ccc(S(=O)(=O)OCC(C)COS(=O)(=O)c2ccc(C)cc2)cc1]

Using General procedure 42 and 2-methylpropane-1,3-diol as the appropriate alcohol, Example 982A was obtained. ¹H NMR (500 MHz, DMSO-d₆) δ ppm: 7.74 (m, 4H), 7.48 (m, 4H), 3.87 (d, 4H), 2.43 (s, 6H), 2.09 (m, 1H), 0.77 (d, 3H). HRMS calculated for C18H₂₂O₆S₂: 398.0858; found 416.1198 (M+NH₄).

### Example 982B methyl (1r,4S,8′S)-4-(3-chloroanilino)-8′-[(2R)-3-hydroxy-2-methylpropyl]-3′-methyl-3′,4′,8′,9′-tetrahydro-2′H-spiro[cyclohexane-1,7′-indeno[5,6-b][1,4]dioxepine]-4-carboxylate

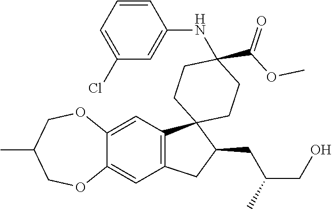 [SMILES: COC(=O)[C@]1(Nc2cccc(Cl)c2)CC[C@@]2(CC1)c1cc3c(cc1C[C@@H]2C[C@@H](C)CO)OCC(C)CO3]

Using General procedure 43 and Preparation 20a as the appropriate catechol and Example 982A as the appropriate bis-tosylate, Example 982B was obtained as a mixture of diastereoisomers. ¹H NMR (400 MHz, DMSO-d₆) δ ppm: 7.06 (t, 1H), 6.93 (s, 1H), 6.78 (s, 1H), 6.58 (t, 1H), 6.56 (dm, 1H), 6.43 (dm, 1H), 6.29 (s, 1H), 4.44 (br t, 1H), 4.16/3.66 (m+m, 4H), 3.64 (s, 3H), 3.21 (br s, 2H), 2.81/2.37 (dd+dd, 2H), 2.40-1.20 (m, 8H), 2.26 (m, 1H), 2.03 (m, 1H), 1.53 (m, 1H), 1.34/1.03 (td+m, 2H), 0.92/0.90 (d/d, 3H), 0.83 (d, 3H). HRMS calculated for C₃₀H₃₈ClNO₅: 527.2438; found 528.2503 (M+H).

### Example 982C methyl (1r,4S,8′S)-4-(3-chloroanilino)-3′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate, diastereoisomer 1

and

### Example 982D methyl (1r,4S,8′S)-4-(3-chloroanilino)-3′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate, diastereoisomer 2

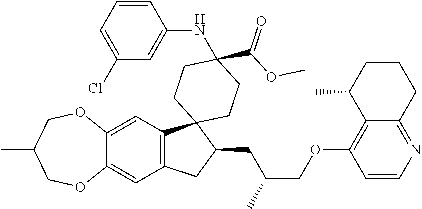 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCC(C)CO3]

Using General procedure 30a and Example 982B as the appropriate indane and Preparation 2a1 as the appropriate aryl-alcohol, a mixture of diastereoisomers was obtained. The diastereoisomers were separated by chiral chromatography. Column: AD, 50×500 mm, 20 μm, Eluents: 30:70 EtOH/heptane. The diastereoisomer eluting earlier was collected as Example 982C. ¹H NMR (500 MHz, DMSO-d₆) δ ppm: 8.14 (d, 1H), 7.04 (t, 1H), 6.94 (s, 1H), 6.78 (s, 1H), 6.77 (d, 1H), 6.57 (t, 1H), 6.55 (dm, 1H), 6.43 (dd, 1H), 6.31 (s, 1H), 4.15/3.69 (m+m, 4H), 3.90/3.85 (dd+dd, 2H), 3.64 (s, 3H), 3.05 (m, 1H), 2.88/2.41 (dd+dd, 2H), 2.76/2.65 (br d+m, 2H), 2.41-1.24 (m, 14H), 2.27 (m, 1H), 2.09 (m, 1H), 1.96 (m, 1H), 1.06 (d, 3H), 1.03 (d, 3H), 0.93 (d, 3H). HRMS calculated for C₄₀H₄₉ClN₂O₅: 672.3333; found 673.3403 (M+H).

The diastereoisomer eluting later was collected as Example 982D. ¹H NMR (500 MHz, DMSO-d₆) δ ppm: 8.14 (d, 1H), 7.04 (t, 1H), 6.94 (s, 1H), 6.79 (s, 1H), 6.76 (d, 1H), 6.57 (t, 1H), 6.55 (dm, 1H), 6.43 (dd, 1H), 6.31 (s, 1H), 4.18/3.63 (m+m, 4H), 3.90/3.84 (dd+dd, 2H), 3.64 (s, 3H), 3.05 (m, 1H), 2.88/2.41 (dd+dd, 2H), 2.76/2.65 (br d+m, 2H), 2.41-1.24 (m, 14H), 2.28 (m, 1H), 2.09 (m, 1H), 1.96 (m, 1H), 1.05 (d, 3H), 1.03 (d, 3H), 0.90 (d, 3H). HRMS calculated for C₄₀H₄₉ClN₂O₅: 672.3333; found 673.3406 (M+H).

### Example 982 (1r,4S,8′S)-4-(3-chloroanilino)-3′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid, diastereoisomer 1

### Example 983 (1r,4S,8′S)-4-(3-chloroanilino)-3′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid, diastereoisomer 2

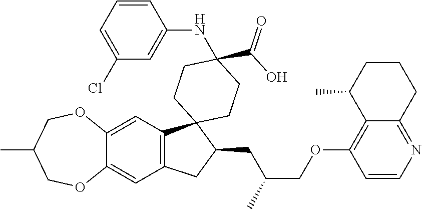 [SMILES: CC1COc2cc3c(cc2OC1)[C@]1(CC[C@@](Nc2cccc(Cl)c2)(C(=O)O)CC1)[C@@H](C[C@@H](C)COc1ccnc2c1[C@H](C)CCC2)C3]

Using General procedure 33a and Example 982C as the appropriate ester, Example 982 was obtained. ¹H NMR (500 MHz, DMSO-d₆) δ ppm: 12.71 (br s, 1H), 8.14 (d, 1H), 7.03 (t, 1H), 6.94 (s, 1H), 6.78 (s, 1H), 6.77 (d, 1H), 6.59 (t, 1H), 6.53 (dd, 1H), 6.51 (dd, 1H), 6.22 (br s, 1H), 4.15/3.69 (m+m, 4H), 3.90/3.85 (dd+dd, 2H), 3.05 (m, 1H), 2.88/2.41 (dd+dd, 2H), 2.76/2.65 (br d+m, 2H), 2.41-1.24 (m, 14H), 2.27 (m, 1H), 2.08 (m, 1H), 1.96 (m, 1H), 1.07 (d, 3H), 1.03 (d, 3H), 0.92 (d, 3H). HRMS calculated for C₃₉H₄₇ClN₂O₅: 658.3174; found 659.3248 (M+H).

Using General procedure 33a and Example 982D as the appropriate ester, Example 983 was obtained. ¹H NMR (500 MHz, DMSO-d₆) δ ppm: 12.71 (br s, 1H), 8.14 (d, 1H), 7.03 (t, 1H), 6.94 (s, 1H), 6.79 (s, 1H), 6.76 (d, 1H), 6.59 (t, 1H), 6.53 (dd, 1H), 6.51 (dd, 1H), 6.23 (br s, 1H), 4.18/3.63 (m+m, 4H), 3.90/3.84 (dd+dd, 2H), 3.06 (m, 1H), 2.88/2.41 (dd+dd, 2H), 2.76/2.65 (br d+m, 2H), 2.41-1.24 (m, 14H), 2.28 (m, 1H), 2.09 (m, 1H), 1.96 (m, 1H), 1.06 (d, 3H), 1.03 (d, 3H), 0.90 (d, 3H). HRMS calculated for C₃₉H₄₇ClN₂O₅: 658.3174; found 659.3251 (M+H).

### Example 984 and Example 985

### Example 984A (3R)-butane-1,3-diyl bis(4-methylbenzene-1-sulfonate)

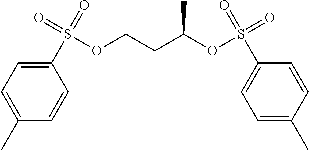 [SMILES: Cc1ccc(S(=O)(=O)OCC[C@@H](C)OS(=O)(=O)c2ccc(C)cc2)cc1]

Using General procedure 42 and (3R)-butane-1,3-diol as the appropriate alcohol, Example 984A was obtained. ¹H NMR (400 MHz, DMSO-d₆) δ ppm: 7.74 (d, 2H), 7.73 (d, 2H), 7.49 (d, 2H), 7.45 (d, 2H), 4.59 (m, 1H), 3.91/3.84 (dt+dt, 2H), 2.43 (s, 3H), 2.42 (s, 3H), 1.86/1.84 (m+m, 2H), 1.13 (d, 3H). HRMS calculated for C₁₈H₂₂O₆S₂: 398.0858; found 416.1191 (M+NH₄).

### Example 984B methyl (1r,4S,4'S,8′S)-4-(3-chloroanilino)-8′-[(2R)-3-hydroxy-2-methylpropyl]-4′-methyl-3′,4′,8′,9′-tetrahydro-2′H-spiro[cyclohexane-1,7′-indeno[5,6-b][1,4]dioxepine]-4-carboxylate

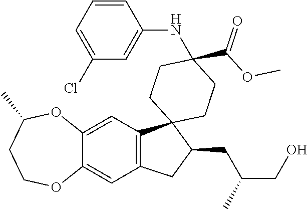 [SMILES: COC(=O)[C@]1(Nc2cccc(Cl)c2)CC[C@@]2(CC1)c1cc3c(cc1C[C@@H]2C[C@@H](C)CO)OCC[C@H](C)O3]

and

### Example 985B methyl (1r,2′S,4S,8′S)-4-(3-chloroanilino)-8′-[(2R)-3-hydroxy-2-methylpropyl]-2′-methyl-3′,4′,8′,9′-tetrahydro-2′H-spiro[cyclohexane-1,7′-indeno[5,6-b][1,4]dioxepine]-4-carboxylate

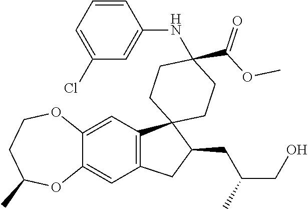 [SMILES: COC(=O)[C@]1(Nc2cccc(Cl)c2)CC[C@@]2(CC1)c1cc3c(cc1C[C@@H]2C[C@@H](C)CO)O[C@@H](C)CCO3]

Using General procedure 43 and Preparation 20a as the appropriate catechol and Example 984A as the appropriate bis-tosylate, a mixture of regioisomers was obtained. The regioisomers were separated via prep RP-HPLC using 25 mM aq. NH₄HCO₃ solution and MeCN as eluents. The regioisomer eluting earlier was collected as Example 984B. ¹H NMR (500 MHz, DMSO-d₆) δ ppm: 7.06 (t, 1H), 6.89 (s, 1H), 6.75 (s, 1H), 6.58 (t, 1H), 6.56 (dm, 1H), 6.44 (dm, 1H), 6.29 (s, 1H), 4.44 (t, 1H), 4.23/3.88 (m+m, 2H), 4.07 (m, 1H), 3.65 (s, 3H), 3.22/3.19 (m+m, 2H), 2.83/2.37 (dd+dd, 2H), 2.40-1.28 (m, 8H), 2.06/1.90 (m+m, 2H), 2.06 (m, 1H), 1.53 (m, 1H), 1.34 (d, 3H), 1.31/1.02 (m+m, 2H), 0.84 (d, 3H). HRMS calculated for C₃₀H₃₈ClNO₅: 527.2438; found 528.2515 (M+H).

The regioisomer eluting later was collected as Example 985B. ¹H NMR (500 MHz, DMSO-d₆) δ ppm: 7.06 (t, 1H), 6.92 (s, 1H), 6.77 (s, 1H), 6.58 (t, 1H), 6.56 (dm, 1H), 6.43 (dm, 1H), 6.29 (s, 1H), 4.45 (t, 1H), 4.27/3.85 (m+m, 2H), 4.01 (m, 1H), 3.64 (s, 3H), 3.21 (m, 2H), 2.81/2.37 (dd+dd, 2H), 2.42-1.20 (m, 8H), 2.05/1.91 (m+m, 2H), 2.01 (m, 1H), 1.53 (m, 1H), 1.35/1.04 (m+m, 2H), 1.33 (d, 3H), 0.83 (d, 3H). HRMS calculated for C₃₀H₃₈ClNO₅: 527.2438; found 528.2512 (M+H).

### Example 984C methyl (1r,4S,4'S,8′S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate

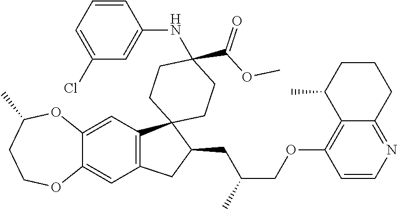 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCC[C@H](C)O3]

Using General procedure 30a and Example 984B as the appropriate indane and Preparation 2a1 as the appropriate aryl-alcohol, Example 984C was obtained. ¹H NMR (500 MHz, DMSO-d₆) δ ppm: 8.14 (d, 1H), 7.04 (t, 1H), 6.90 (s, 1H), 6.76 (d, 1H), 6.76 (s, 1H), 6.58 (t, 1H), 6.56 (dd, 1H), 6.44 (dd, 1H), 6.30 (s, 1H), 4.23/3.86 (m+m, 2H), 4.05 (m, 1H), 3.88/3.84 (dd+dd, 2H), 3.65 (s, 3H), 3.04 (m, 1H), 2.89/2.41 (dd+dd, 2H), 2.76/2.66 (m+m, 2H), 2.37-1.21 (m, 8H), 2.12 (m, 1H), 2.06/1.90 (m+m, 2H), 1.96 (m, 1H), 1.79/1.74 (m+m, 2H), 1.67/1.61 (m+m, 2H), 1.45/1.29 (t+t, 2H), 1.34 (d, 3H), 1.05 (d, 3H), 1.03 (d, 3H). HRMS calculated for C₄₀H₄₉ClN₂O₅: 672.3333; found 673.3405 (M+H).

### Example 984 (1r,4S,4'S,8′S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

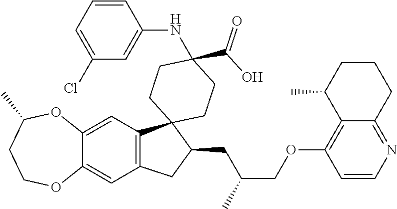 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)O[C@@H](C)CCO3]

Using General procedure 33a and Example 984C as the appropriate ester, Example 984 was obtained. ¹H NMR (500 MHz, DMSO-d₆) δ ppm: 12.64 (br s, 1H), 8.14 (d, 1H), 7.04 (t, 1H), 6.90 (s, 1H), 6.77 (d, 1H), 6.76 (s, 1H), 6.60 (t, 1H), 6.54 (dd, 1H), 6.52 (dd, 1H), 6.23 (br s, 1H), 4.23/3.87 (m+m, 2H), 4.05 (m, 1H), 3.89/3.84 (dd+dd, 2H), 3.05 (m, 1H), 2.89/2.41 (dd+dd, 2H), 2.76/2.65 (m+m, 2H), 2.37-1.32 (m, 8H), 2.12 (m, 1H), 2.06/1.91 (m+m, 2H), 1.96 (m, 1H), 1.80/1.74 (m+m, 2H), 1.68/1.61 (m+m, 2H), 1.46/1.31 (t+t, 2H), 1.33 (d, 3H), 1.05 (d, 3H), 1.03 (d, 3H). HRMS calculated for C₃₉H₄₇ClN₂O₅: 658.3174; found 659.3255 (M+H).

### Example 985C methyl (1r,2′S,4S,8′S)-4-(3-chloroanilino)-2′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate

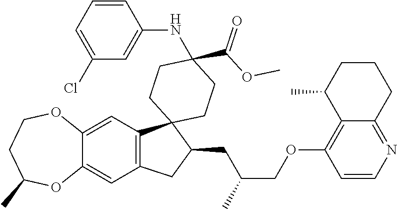 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)O[C@@H](C)CCO3]

Using General procedure 30a and Example 985B as the appropriate indane and Preparation 2a1 as the appropriate aryl-alcohol, Example 985C was obtained. ¹H NMR (500 MHz, DMSO-d₆) δ ppm: 8.14 (d, 1H), 7.04 (t, 1H), 6.93 (s, 1H), 6.78 (s, 1H), 6.76 (d, 1H), 6.57 (t, 1H), 6.55 (dd, 1H), 6.43 (dd, 1H), 6.31 (s, 1H), 4.27/3.96 (m+m, 2H), 4.01 (m, 1H), 3.90/3.84 (dd+dd, 2H), 3.64 (s, 3H), 3.06 (m, 1H), 2.88/2.41 (dd+dd, 2H), 2.76/2.66 (m+m, 2H), 2.40-1.23 (m, 8H), 2.08 (m, 1H), 2.06/1.92 (m+m, 2H), 1.96 (m, 1H), 1.80/1.74 (m+m, 2H), 1.68/1.62 (m+m, 2H), 1.50/1.32 (t+t, 2H), 1.33 (d, 3H), 1.06 (d, 3H), 1.02 (d, 3H). HRMS calculated for C₄₀H₄₉ClN₂O₅: 672.3333; found 673.3401 (M+H).

### Example 985 (1r,2′S,4S,8′S)-4-(3-chloroanilino)-2′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

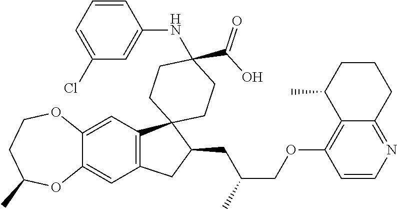 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC[C@H](C)O3]

Using General procedure 33a and Example 985C as the appropriate ester, Example 985 was obtained. ¹H NMR (500 MHz, DMSO-d₆) δ ppm: 12.63 (br s, 1H), 8.14 (d, 1H), 7.04 (t, 1H), 6.94 (s, 1H), 6.78 (s, 1H), 6.77 (d, 1H), 6.59 (t, 1H), 6.53 (dd, 1H), 6.51 (dd, 1H), 6.20 (br s, 1H), 4.26/3.86 (m+m, 2H), 4.01 (m, 1H), 3.91/3.85 (dd+dd, 2H), 3.06 (m, 1H), 2.88/2.41 (dd+dd, 2H), 2.76/2.65 (m+m, 2H), 2.39-1.23 (m, 8H), 2.07 (m, 1H), 2.06/1.91 (m+m, 2H), 1.97 (m, 1H), 1.81/1.74 (m+m, 2H), 1.68/1.62 (m+m, 2H), 1.50/1.33 (t+t, 2H), 1.33 (d, 3H), 1.07 (d, 3H), 1.02 (d, 3H). HRMS calculated for C₃₉H₄₇ClN₂O₅: 658.3174; found 659.3253 (M+H).

### Example 986 and Example 987

### Example 986A (3S)-butane-1,3-diyl bis(4-methylbenzene-1-sulfonate)

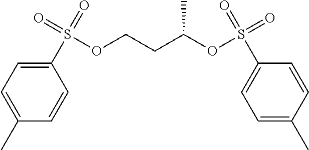 [SMILES: Cc1ccc(S(=O)(=O)OCC[C@H](C)OS(=O)(=O)c2ccc(C)cc2)cc1]

Using General procedure 42 and (3S)-butane-1,3-diol as the appropriate alcohol, Example 986A was obtained. ¹H NMR (400 MHz, DMSO-d₆) δ ppm: 7.74 (d, 2H), 7.73 (d, 2H), 7.49 (d, 2H), 7.45 (d, 2H), 4.59 (m, 1H), 3.91/3.84 (dt+dt, 2H), 2.43 (s, 3H), 2.42 (s, 3H), 1.86/1.84 (m+m, 2H), 1.13 (d, 3H). HRMS calculated for C₁₈H₂₂O₆S₂: 398.0858; found 416.1191 (M+NH₄).

### Example 986B methyl (1r,2′R,4S,8′S)-4-(3-chloroanilino)-8′-[(2R)-3-hydroxy-2-methylpropyl]-2′-methyl-3′,4′,8′,9′-tetrahydro-2′H-spiro[cyclohexane-1,7′-indeno[5,6-b][1,4]dioxepine]-4-carboxylate and methyl (1r,4S,4′R,8′S)-4-(3-chloroanilino)-8′-[(2R)-3-hydroxy-2-methylpropyl]-4′-methyl-3′,4′,8′,9′-tetrahydro-2′H-spiro[cyclohexane-1,7′-indeno[5,6-b][1,4]dioxepine]-4-carboxylate

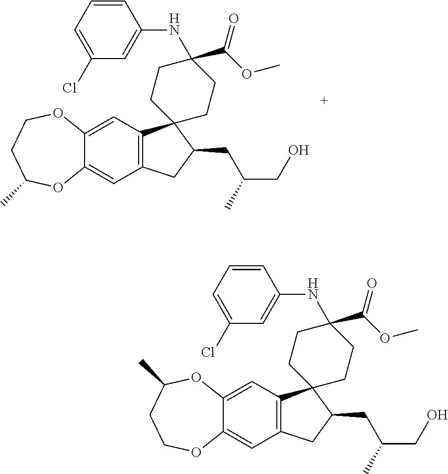 [SMILES: COC(=O)[C@]1(Nc2cccc(Cl)c2)CC[C@@]2(CC1)c1cc3c(cc1C[C@@H]2C[C@@H](C)CO)O[C@H](C)CCO3 ; COC(=O)[C@]1(Nc2cccc(Cl)c2)CC[C@@]2(CC1)c1cc3c(cc1C[C@@H]2C[C@@H](C)CO)OCC[C@@H](C)O3]

Using General procedure 43 and Preparation 20a as the appropriate catechol and Example 986A as the appropriate bis-tosylate, Example 986B was obtained as a mixture of regioisomers. ¹H NMR (400 MHz, DMSO-d₆) δ ppm: 7.06 (t, 1H), 6.91/6.90 (s/s, 1H), 6.77/6.75 (s/s, 1H), 6.58 (m, 1H), 6.56 (dm, 1H), 6.44 (dd, 1H), 6.29 (s, 1H), 4.45 (t, 1H), 4.23/3.88 (m+m, 2H), 4.07 (m, 1H), 3.65 (s, 3H), 3.22/3.19 (m+m, 2H), 2.83/2.37 (dd+dd, 2H), 2.40-1.28 (m, 8H), 2.06/1.9 (m+m, 2H), 2.06 (m, 1H), 1.53 (m, 1H), 1.34 (d, 3H), 1.31/1.02 (m+m, 2H), 0.84 (d, 3H). HRMS calculated for C₃₀H₃₈ClNO₅: 527.2438; found 528.2514 (M+H).

### Example 986C methyl (1r,2′R,4S,8′S)-4-(3-chloroanilino)-2′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate

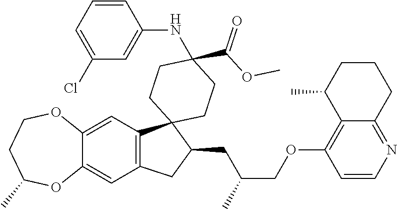 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)O[C@H](C)CCO3]

and

### Example 987C methyl (1r,4S,4′R,8′S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylate

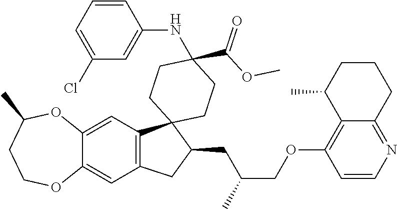 [SMILES: COC(=O)[C@]1(Nc2cccc(Cl)c2)CC[C@@]2(CC1)c1cc3c(cc1C[C@@H]2C[C@@H](C)COc1ccnc2c1[C@H](C)CCC2)OCC[C@@H](C)O3]

Using General procedure 30a and Example 986B as the appropriate indane and Preparation 2a1 as the appropriate aryl-alcohol, a mixture of regioisomers was obtained. The regioisomers were separated by chiral chromatography. Column: AD, 50×500 mm, 20 μm, Eluents: 15:85 EtOH/heptane. The regioisomer eluting earlier was collected as Example 986C. ¹H NMR (500 MHz, DMSO-d₆) δ ppm: 8.14 (d, 1H), 7.04 (t, 1H), 6.91 (s, 1H), 6.772 (d, 1H), 6.770 (s, 1H), 6.57 (t, 1H), 6.55 (dd, 1H), 6.43 (dd, 1H), 6.31 (s, 1H), 4.25/3.91 (m+m, 2H), 4.08 (m, 1H), 3.89/3.85 (dd+dd, 2H), 3.64 (s, 3H), 3.05 (m, 1H), 2.88/2.41 (dd+dd, 2H), 2.76/2.65 (m+m, 2H), 2.40-1.24 (m, 8H), 2.11 (m, 1H), 2.08/1.91 (m+m, 2H), 1.96 (m, 1H), 1.80/1.74 (m+m, 2H), 1.68/1.62 (m+m, 2H), 1.47/1.30 (t+t, 2H), 1.32 (d, 3H), 1.05 (d, 3H), 1.03 (d, 3H). HRMS calculated for C₄₀H₄₉ClN₂O₅: 672.3333; found 673.3409 (M+H).

The regioisomer eluting later was collected as Example 987C. ¹H NMR (500 MHz, DMSO-d₆) δ ppm: 8.14 (d, 1H), 7.04 (t, 1H), 6.92 (s, 1H), 6.77 (d, 1H), 6.76 (s, 1H), 6.57 (t, 1H), 6.55 (dd, 1H), 6.43 (dd, 1H), 6.31 (s, 1H), 4.25/3.87 (m+m, 2H), 4.04 (m, 1H), 3.90/3.85 (dd+dd, 2H), 3.64 (s, 3H), 3.05 (m, 1H), 2.87/2.42 (dd+dd, 2H), 2.76/2.66 (m+m, 2H), 2.36-1.24 (m, 8H), 2.09 (m, 1H), 2.07/1.91 (m+m, 2H), 1.97 (m, 1H), 1.80/1.74 (m+m, 2H), 1.68/1.62 (m+m, 2H), 1.46/1.31 (t+t, 2H), 1.35 (d, 3H), 1.05 (d, 3H), 1.03 (d, 3H). HRMS calculated for C₄₀H₄₉ClN₂O₅: 672.3333; found 673.3404 (M+H).

### Example 986 (1r,2′R,4S,8′S)-4-(3-chloroanilino)-2′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

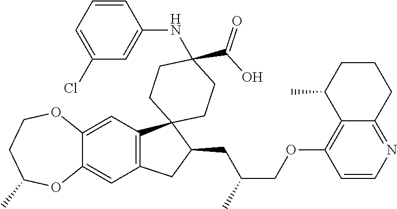 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC[C@@H](C)O3]

Using General procedure 33a and Example 986C as the appropriate ester, Example 986 was obtained. ¹H NMR (500 MHz, DMSO-d₆) δ ppm: 12.69 (br s, 1H), 8.14 (d, 1H), 7.03 (t, 1H), 6.91 (s, 1H), 6.774 (s, 1H), 6.772 (d, 1H), 6.59 (t, 1H), 6.53 (dm, 1H), 6.51 (dm, 1H), 6.22 (br s, 1H), 4.25/3.91 (m+m, 2H), 4.07 (m, 1H), 3.89/3.85 (dd+dd, 2H), 3.06 (m, 1H), 2.88/2.41 (dd+dd, 2H), 2.76/2.66 (m+m, 2H), 2.41-1.23 (m, 14H), 2.09 (m, 1H), 1.96 (m, 1H), 1.48/1.32 (m+m, 2H), 1.32 (d, 3H), 1.06 (d, 3H), 1.02 (d, 3H). HRMS calculated for C₃₉H₄₇ClN₂O₅: 658.3174; found 659.3243 (M+H).

### Example 987 (1r,4S,4′R,8′S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

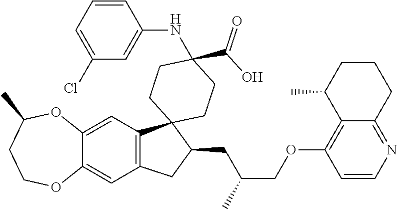 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)O[C@H](C)CCO3]

Using General procedure 33a and Example 987C as the appropriate ester, Example 987 was obtained. ¹H NMR (500 MHz, DMSO-d₆) δ ppm: 12.70 (br s, 1H), 8.14 (d, 1H), 7.04 (t, 1H), 6.92 (s, 1H), 6.77 (d, 1H), 6.76 (s, 1H), 6.60 (t, 1H), 6.54 (dd, 1H), 6.52 (dd, 1H), 6.22 (br s, 1H), 4.25/3.86 (m+m, 2H), 4.03 (m, 1H), 3.90/3.85 (dd+dd, 2H), 3.05 (m, 1H), 2.87/2.42 (dd+dd, 2H), 2.76/2.65 (m+m, 2H), 2.40-1.24 (m, 8H), 2.09 (m, 1H), 2.06/1.91 (m+m, 2H), 1.96 (m, 1H), 1.80/1.74 (m+m, 2H), 1.68/1.62 (m+m, 2H), 1.49/1.31 (t+t, 2H), 1.35 (d, 3H), 1.05 (d, 3H), 1.03 (d, 3H). HRMS calculated for C₃₉H₄₇ClN₂O₅: 658.3174; found 659.3251 (M+H).

### Example 988 and Example 989

### Example 988A 2-{[(4-methylbenzene-1-sulfonyl)oxy]methyl}butyl 4-methylbenzene-1-sulfonate

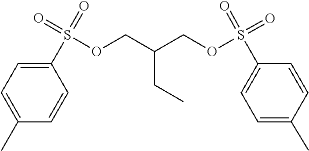

Using General procedure 42 and 2-ethylpropane-1,3-diol as the appropriate alcohol, Example 988A was obtained. ¹H NMR (500 MHz, DMSO-d₆) δ ppm: 7.74 (m, 4H), 7.48 (m, 4H), 3.92/3.89 (dd+dd, 4H), 2.43 (s, 6H), 1.88 (m, 1H), 1.20 (m, 2H), 0.67 (t, 3H). HRMS calculated for C₁₉H₂₄O₆S₂: 412.1014; found 435.0908 (M+Na).

### Example 988 (1r,4S,8′S)-4-(3-chloroanilino)-3′-ethyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid, diastereoisomer 1

and

### Example 989 (1r,4S,8′S)-4-(3-chloroanilino)-3′-ethyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid, diastereoisomer 2

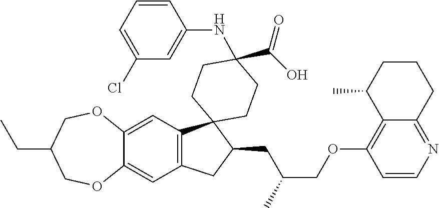 [SMILES: CCC1COc2cc3c(cc2OC1)[C@]1(CC[C@@](Nc2cccc(Cl)c2)(C(=O)O)CC1)[C@@H](C[C@@H](C)COc1ccnc2c1[C@H](C)CCC2)C3]

Using General Procedure 43 and Preparation 20b as the appropriate catechol and Example 988A as the appropriate bis-tosylate, a mixture of diastereoisomers was obtained. The diastereoisomers were separated by chiral chromatography. Column: IG, 100×500 mm, 20 μm, Eluents: 50:50 EtOH/heptane. The diastereoisomer eluting earlier was hydrolyzed according to General Procedure 33a to obtain Example 988. ¹H NMR (500 MHz, DMSO-d₆) δ ppm: 12.71 (br s, 1H), 8.14 (d, 1H), 7.03 (t, 1H), 6.93 (s, 1H), 6.77 (d, 1H), 6.76 (s, 1H), 6.59 (t, 1H), 6.53 (dm, 1H), 6.51 (dm, 1H), 6.22 (br s, 1H), 4.15/3.86 (m+m, 4H), 3.90/3.84 (dd+dd, 2H), 3.05 (m, 1H), 2.87/2.40 (dd+dd, 2H), 2.76/2.65 (br d+m, 2H), 2.40-1.21 (m, 14H), 2.07 (m, 1H), 2.02 (m, 1H), 1.96 (m, 1H), 1.37 (m, 2H), 1.06 (d, 3H), 1.02 (d, 3H), 0.94 (t, 3H). HRMS calculated for C₄₀H₄₉ClN₂O₅: 672.3330; found: 673.3405 (M+H).

The diastereoisomer eluting later was hydrolyzed according to General Procedure 33a to obtain Example 989. HRMS calculated for C₄₀H₄₉ClN₂O₅: 672.3330; found: 673.3408 (M+H).

### Example 990 and Example 991

### Example 990A 2-{[(4-methylbenzene-1-sulfonyl)oxy]methyl}pentyl 4-methylbenzene-1-sulfonate

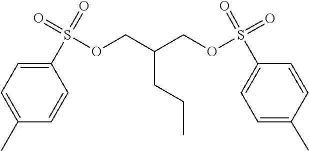

Using General procedure 42 and 2-propylpropane-1,3-diol as the appropriate alcohol, Example 990A was obtained. ¹H NMR (500 MHz, DMSO-d₆) δ ppm: 7.74 (d, 4H), 7.48 (d, 4H), 3.91/3.88 (dd+dd, 4H), 2.43 (s, 6H), 1.94 (m, 1H), 1.11 (m, 2H), 1.04 (m, 2H), 0.71 (t, 3H). HRMS calculated for C₂₀H₂₆O6S₂: 426.1171; found 427.1246 (M+H).

### Example 990 (1r,4S,8′S)-4-(3-chloroanilino)-8′-[(2R)-2-methyl-3-{[(5R)-5-methyl-5,6,7,8-tetrahydroquinolin-4-yl]oxy}propyl]-3′-propyl-3′,4′,8′,9′-tetrahydro-2′H-spiro[cyclohexane-1,7′-indeno[5,6-b][1,4]dioxepine]-4-carboxylic acid, diastereoisomer 1

and

### Example 991 (1r,4S,8′S)-4-(3-chloroanilino)-8′-[(2R)-2-methyl-3-{[(5R)-5-methyl-5,6,7,8-tetrahydroquinolin-4-yl]oxy}propyl]-3′-propyl-3′,4′,8′,9′-tetrahydro-2′H-spiro[cyclohexane-1,7′-indeno[5,6-b][1,4]dioxepine]-4-carboxylic acid, diastereoisomer 2

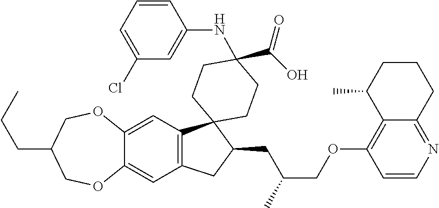 [SMILES: CCCC1COc2cc3c(cc2OC1)[C@]1(CC[C@@](Nc2cccc(Cl)c2)(C(=O)O)CC1)[C@@H](C[C@@H](C)COc1ccnc2c1[C@H](C)CCC2)C3]

Using General Procedure 43 and Preparation 20b as the appropriate catechol and Example 990A as the appropriate bis-tosylate, a mixture of diastereoisomers was obtained. The diastereoisomers were separated by chiral chromatography. Column: IG, 100×500 mm, 20 μm, Eluents: 50:50 EtOH/heptane. The diastereoisomer eluting earlier was hydrolyzed according to General Procedure 33a to obtain Example 990. ¹H NMR (500 MHz, DMSO-d₆) δ ppm: 12.72 (br s, 1H), 8.18 (d, 1H), 7.03 (t, 1H), 6.93 (s, 1H), 6.77 (d, 1H), 6.76 (s, 1H), 6.59 (t, 1H), 6.53 (dd, 1H), 6.51 (dd, 1H), 6.22 (br s, 1H), 4.15/3.84 (m+m, 4H), 3.90/3.84 (dd+dd, 2H), 3.05 (m, 1H), 2.87/2.40 (dd+dd, 2H), 2.76/2.65 (m, 2H), 2.40-1.20 (m, 18H), 2.11 (m, 1H), 2.07 (m, 1H), 1.96 (m, 1H), 1.06 (d, 3H), 1.02 (d, 3H), 0.90 (t, 3H). HRMS calculated for C₄₁H₅₁ClN₂O₅: 686.3486; found: 687.3561 (M+H).

The diastereoisomer eluting later was hydrolyzed according to General Procedure 33a to obtain Example 991. HRMS calculated for C₄₁H₅₁ClN₂O₅: 686.3486; found: 687.3562 (M+H).

### Example 992 and Example 993

### Example 992A 3-methyl-2-{[(4-methylbenzene-1-sulfonyl)oxy]methyl}butyl 4-methylbenzene-1-sulfonate

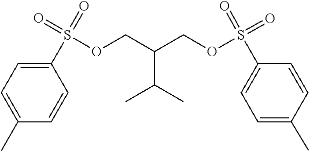

Using General procedure 42 and 2-isopropylpropane-1,3-diol as the appropriate alcohol, Example 992A was obtained. ¹H NMR (500 MHz, DMSO-d₆) δ ppm: 7.74 (dm, 4H), 7.48 (dm, 4H), 3.99/3.92 (dd+dd, 4H), 2.43 (s, 6H), 1.76 (m, 1H), 1.61 (m, 1H), 0.72 (d, 6H). HRMS calculated for C₂₀H₂₆O6S₂: 426.1171; found 449.1067 (M+Na).

### Example 992 (1r,4S,8′S)-4-(3-chloroanilino)-8′-[(2R)-2-methyl-3-{[(5R)-5-methyl-5,6,7,8-tetrahydroquinolin-4-yl]oxy}propyl]-3′-(propan-2-yl)-3′,4′,8′,9′-tetrahydro-2′H-spiro[cyclohexane-1,7′-indeno[5,6-b][1,4]dioxepine]-4-carboxylic acid, diastereoisomer 1

and

### Example 993 (1r,4S,8′S)-4-(3-chloroanilino)-8′-[(2R)-2-methyl-3-{[(5R)-5-methyl-5,6,7,8-tetrahydroquinolin-4-yl]oxy}propyl]-3′-(propan-2-yl)-3′,4′,8′,9′-tetrahydro-2′H-spiro[cyclohexane-1,7′-indeno[5,6-b][1,4]dioxepine]-4-carboxylic acid, diastereoisomer 2

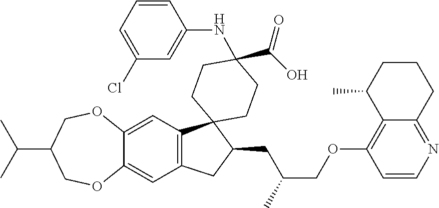 [SMILES: CC(C)C1COc2cc3c(cc2OC1)[C@]1(CC[C@@](Nc2cccc(Cl)c2)(C(=O)O)CC1)[C@@H](C[C@@H](C)COc1ccnc2c1[C@H](C)CCC2)C3]

Using General Procedure 43 and Preparation 20b as the appropriate catechol and Example 992A as the appropriate bis-tosylate, a mixture of diastereoisomers was obtained. The diastereoisomers were separated by chiral chromatography. Column: IG, 100×500 mm, 20 μm, Eluents: 30:70 EtOH/heptane. The diastereoisomer eluting earlier was hydrolyzed according to General Procedure 33a to obtain Example 992. ¹H NMR (500 MHz, DMSO-d₆) δ ppm: 12.7 (br s, 1H), 8.14 (d, 1H), 7.03 (t, 1H), 6.90 (s, 1H), 6.77 (d, 1H), 6.73 (s, 1H), 6.59 (t, 1H), 6.52 (dm, 1H), 6.51 (dm, 1H), 6.22 (br s, 1H), 4.17/4.07 (dd+dd, 4H), 3.90/3.84 (dd+dd, 2H), 3.05 (m, 1H), 2.86/2.40 (dd+dd, 2H), 2.76/2.65 (m+m, 2H), 2.41-1.19 (m, 14H), 2.07 (m, 1H), 1.96 (m, 1H), 1.90 (m, 1H), 1.77 (m, 1H), 1.06 (d, 3H), 1.02 (d, 3H), 0.94 (d, 6H). HRMS calculated for C₄₁H₅₁ClN₂O₅: 686.3486; found: 687.3565 (M+H).

The diastereoisomer eluting later was hydrolyzed according to General Procedure 33a to obtain Example 993. HRMS calculated for C₄₁H₅₁ClN₂O₅: 686.3486; found: 687.3564 (M+H).

### Example 994

### Example 994A (cyclopropane-1,1-diyl)bis(methylene) bis(4-methylbenzene-1-sulfonate)

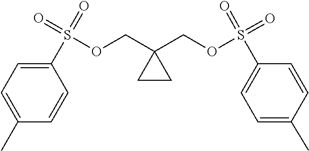

Using General procedure 42 and (cyclopropane-1,1-diyl)dimethanol as the appropriate alcohol, Example 994A was obtained. ¹H NMR (500 MHz, DMSO-d₆) δ ppm: 7.72 (d, 4H), 7.46 (d, 4H), 3.87 (s, 4H), 2.42 (s, 6H), 0.57 (s, 4H). HRMS calculated for C₁₉H₂₂O₆S₂: 410.0858; found 433.0751 (M+Na).

### Example 994 (1r,4S,8′S)-4-(3-chloroanilino)-8′-[(2R)-2-methyl-3-{[(5R)-5-methyl-5,6,7,8-tetrahydroquinolin-4-yl]oxy}propyl]-8′,9′-dihydro-2′H,4′H-dispiro[cyclohexane-1,7′-indeno[5,6-b][1,4]dioxepine-3′,1″-cyclopropane]-4-carboxylic acid

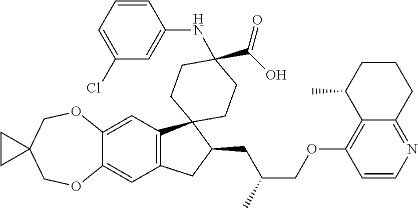 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC1(CC1)CO3]

Using General Procedure 43 and Preparation 20b as the appropriate catechol and Example 994A as the appropriate bis-tosylate, an intermediate was formed which was hydrolyzed according to General Procedure 33a to obtain Example 994. ¹H NMR (500 MHz, DMSO-d₆) δ ppm: 14.65 (br s, 1H), 12.74 (brs, 1H), 8.61 (d, 1H), 7.45 (d, 1H), 7.04 (t, 1H), 6.98 (s, 1H), 6.83 (s, 1H), 6.60 (t, 1H), 6.54 (dm, 1H), 6.51 (dm, 1H), 6.28 (br s, 1H), 4.24/4.17 (dd+dd, 2H), 3.88-3.74 (d+d, 4H), 3.12 (m, 1H), 2.97/2.88 (m+m, 2H), 2.92/2.44 (dd+dd, 2H), 2.45-1.14 (m, 14H), 2.12 (m, 1H), 2.05 (m, 1H), 1.10 (d, 3H), 1.06 (d, 3H), 0.68-0.56 (m, 4H). HRMS calculated for C₄₀H₄₇ClN₂O₅: 670.3173; found: 670.3174 (M+H).

### Example 995

### Example 995A (cyclobutane-1,1-diyl)bis(methylene) bis(4-methylbenzene-1-sulfonate)

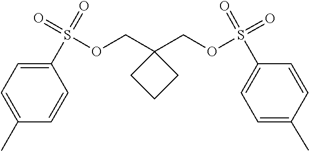

Using General procedure 42 and (cyclobutane-1,1-diyl)dimethanol as the appropriate alcohol, Example 995A was obtained. ¹H NMR (500 MHz, DMSO-d₆) δ ppm: 7.75 (d, 4H), 7.48 (d, 4H), 3.94 (s, 4H), 2.43 (s, 6H), 1.80-1.64 (m, 6H). HRMS calculated for C₂₀H₂₄O₆S₂: 424.1014; found 463.0647 (M+K).

### Example 995 (1″r,4″S,8′S)-4″-(3-chloroanilino)-8′-[(2R)-2-methyl-3-{[(5R)-5-methyl-5,6,7,8-tetrahydroquinolin-4-yl]oxy}propyl]-8′,9′-dihydro-2′H,4′H-dispiro[cyclobutane-1,3′-indeno[5,6-b][1,4]dioxepine-7′,1″-cyclohexane]-4″-carboxylic acid

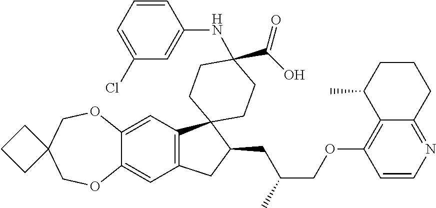 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC1(CCC1)CO3]

Using General Procedure 43 and Preparation 20b as the appropriate catechol and Example 995A as the appropriate bis-tosylate, an intermediate was formed which was hydrolyzed according to General Procedure 33a to obtain Example 995. ¹H NMR (500 MHz, DMSO-d₆) δ ppm: 14.66 (br s, 1H), 12.73 (br s, 1H), 8.61 (d, 1H), 7.44 (d, 1H), 7.04 (t, 1H), 6.95 (s, 1H), 6.79 (s, 1H), 6.59 (t, 1H), 6.54 (dm, 1H), 6.50 (dm, 1H), 6.27 (br s, 1H), 4.23/4.16 (dd+dd, 2H), 4.05-3.90 (d+d, 4H), 3.11 (m, 1H), 2.96/2.88 (m+m, 2H), 2.89/2.42 (dd+dd, 2H), 2.45-1.20 (m, 20H), 2.10 (m, 1H), 2.04 (m, 1H), 1.10 (d, 3H), 1.05 (d, 3H). HRMS calculated for C₄₁H₄₉ClN₂O₅: 684.3330; found: 685.3402 (M+H).

### Example 996

### Example 996A 2,2-difluoropropane-1,3-diyl bis(4-methylbenzene-1-sulfonate)

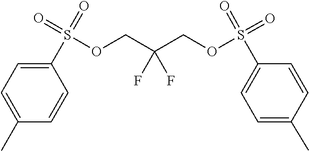

Using General procedure 42 and 2,2-difluoropropane-1,3-diol as the appropriate alcohol, Example 996A was obtained. ¹H NMR (500 MHz, DMSO-d₆) δ ppm: 7.79 (m, 4H), 7.50 (m, 4H), 4.41 (t, 4H), 2.43 (s, 6H). HRMS calculated for C₁₇H₁₈F₂O₆S₂: 420.0513; found 443.0408 (M+Na).

### Example 996 (1r,4S,8′S)-4-(3-chloroanilino)-3′,3′-difluoro-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid

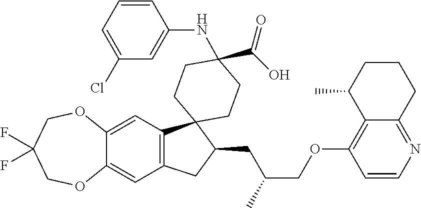 [SMILES: C[C@@H](COc1ccnc2c1[C@H](C)CCC2)C[C@H]1Cc2cc3c(cc2[C@]12CC[C@@](Nc1cccc(Cl)c1)(C(=O)O)CC2)OCC(F)(F)CO3]

Using General Procedure 43 and Preparation 20b as the appropriate catechol and Example 996A as the appropriate bis-tosylate, an intermediate was obtained which was hydrolyzed according to General Procedure 33a to obtain Example 996. ¹H NMR (500 MHz, DMSO-d₆) δ ppm: 12.71 (br s, 1H), 8.14 (d, 1H), 7.03 (t, 1H), 7.02 (s, 1H), 6.89 (s, 1H), 6.77 (d, 1H), 6.59 (t, 1H), 6.53 (dd, 1H), 6.51 (dd, 1H), 6.22 (br s, 1H), 4.52-4.34 (m, 4H), 3.91/3.85 (dd+dd, 2H), 3.05 (m, 1H), 2.92/2.44 (dd+dd, 2H), 2.77/2.65 (br d+m, 2H), 2.44-1.21 (m, 14H), 2.10 (m, 1H), 1.96 (m, 1H), 1.07 (d, 3H), 1.03 (d, 3H). HRMS calculated for C₃₈H₄₃ClF₂N₂O₅: 680.2828; found 681.2908 (M+H).

### Example 997 and Example 998

### Example 997A (3R)-3-[(methanesulfonyl)oxy]-4-methylpentyl 4-methylbenzene-1-sulfonate

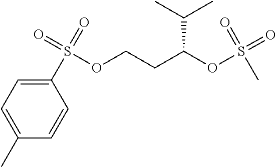 [SMILES: Cc1ccc(S(=O)(=O)OCC[C@@H](OS(C)(=O)=O)C(C)C)cc1]

To a solution of (3R)-4-methylpentane-1,3-diol (250 mg, 2.12 mmol, 1 eq.) in DCM (4.2 mL) was added DMAP (25.9 mg, 0.212 mmol, 0.1 eq), TEA (1.03 mL, 7.40 mmol, 3.5 eq) and TsCl (1.41 g, 7.40 mmol, 3.5 eq). The mixture was stirred at 40° C. for 1 h. Then, it was cooled to 0° C. and MsCl (327 L, 4.23 mmol, 2.0 eq) was added. The mixture was stirred at 0° C. until no further conversion was observed. Then it was quenched with 2 M aq. HCl solution and the layers were separated. The organic layer was dried over Na₂SO₄, filtered and the filtrate was concentrated under reduced pressure. The crude product was purified via flash chromatography using heptane and EtOAc as eluents to obtain Example 997A (635 mg, 1.81 mmol, 86%). ¹H NMR (500 MHz, DMSO-d₆) δ ppm: 7.80 (d, 2H), 7.49 (d, 2H), 4.51 (m, 1H), 4.18-4.07 (m, 2H), 3.13 (s, 3H), 2.43 (s, 3H), 1.99-1.85 (m, 2H), 1.99-1.85 (m, 1H), 0.86/0.82 (d+d, 6H). HRMS calculated for C₁₄H₂₂O₆S₂: 350.0588; found: 368.1197 (M+NH₄).

### Example 997 (1r,2′S,4S,8′S)-4-(3-chloroanilino)-8′-[(2R)-2-methyl-3-{[(5R)-5-methyl-5,6,7,8-tetrahydroquinolin-4-yl]oxy}propyl]-2′-(propan-2-yl)-3′,4′,8′,9′-tetrahydro-2′H-spiro[cyclohexane-1,7′-indeno[5,6-b][1,4]dioxepine]-4-carboxylic acid

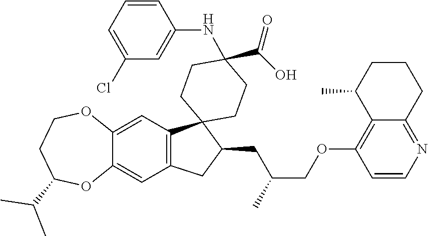 [SMILES: CC(C)[C@@H]1CCOc2cc3c(cc2O1)C[C@H](C[C@@H](C)COc1ccnc2c1[C@H](C)CCC2)[C@]31CC[C@](Nc2cccc(Cl)c2)(C(=O)O)CC1]

and

### Example 998 (1r,4S,4'S,8′S)-4-(3-chloroanilino)-8′-[(2R)-2-methyl-3-{[(5R)-5-methyl-5,6,7,8-tetrahydroquinolin-4-yl]oxy}propyl]-4′-(propan-2-yl)-3′,4′,8′,9′-tetrahydro-2′H-spiro[cyclohexane-1,7′-indeno[5,6-b][1,4]dioxepine]-4-carboxylic acid

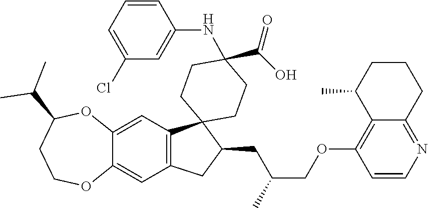 [SMILES: CC(C)[C@@H]1CCOc2cc3c(cc2O1)[C@]1(CC[C@@](Nc2cccc(Cl)c2)(C(=O)O)CC1)[C@@H](C[C@@H](C)COc1ccnc2c1[C@H](C)CCC2)C3]

Using General procedure 43 and Preparation 20b as the appropriate cathecol and Example 997A instead the appropriate bis-tosylate, a mixture of regioisomers was obtained. The regioisomers were separated by chiral chromatography. Column: AD, 50×500 mm, 20 μm, Eluents: 20:80 PrOH/heptane. The regioisomer eluting earlier was hydrolyzed according to General Procedure 33a to obtain Example 997. ¹H NMR (500 MHz, DMSO-d₆) δ ppm: 12.70 (br s, 1H), 8.14 (d, 1H), 7.03 (t, 1H), 6.89 (s, 1H), 6.77 (s, 1H), 6.76 (d, 1H), 6.59 (t, 1H), 6.53 (dd, 1H), 6.51 (dd, 1H), 6.22 (br s, 1H), 4.28/3.95 (m+m, 2H), 3.90/3.84 (dd+dd, 2H), 3.64 (m, 1H), 3.06 (m, 1H), 2.88/2.42 (dd+dd, 2H), 2.76/2.65 (m+m, 2H), 2.38-1.25 (m, 8H), 2.09 (m, 1H), 2.08/1.93 (m+m, 2H), 1.97 (m, 1H), 1.82 (m, 1H), 1.80/1.74 (m+m, 2H), 1.68/1.62 (m+m, 2H), 1.48/1.31 (t+t, 2H), 1.06 (d, 3H), 1.03 (d, 3H), 1.01/0.95 (d+d, 6H). HRMS calculated for C₄₁H₅₁ClN₂O₅: 686.3487; found: 687.3563 (M+H).

The regioisomer eluting later was hydrolyzed according to General procedure 33a to obtain Example 998. ¹H NMR (500 MHz, DMSO-d₆) δ ppm: 12.70 (br s, 1H), 8.14 (d, 1H), 7.03 (t, 1H), 6.94 (s, 1H), 6.77 (d, 1H), 6.74 (s, 1H), 6.59 (t, 1H), 6.53 (dd, 1H), 6.52 (dd, 1H), 6.22 (br s, 1H), 4.29/3.93 (m+m, 2H), 3.90/3.84 (dd+dd, 2H), 3.60 (m, 1H), 3.05 (m, 1H), 2.87/2.42 (dd+dd, 2H), 2.76/2.65 (m+m, 2H), 2.39-1.26 (m, 8H), 2.09 (m, 1H), 2.06/1.93 (m+m, 2H), 1.96 (m, 1H), 1.84 (m, 1H), 1.80/1.74 (m+m, 2H), 1.68/1.62 (m+m, 2H), 1.49/1.32 (t+t, 2H), 1.06 (d, 3H), 1.02/0.95 (d+d, 6H), 1.02 (d, 3H). HRMS calculated for C₄₁H₅₁ClN₂O₅: 686.3487; found: 687.3561 (M+H).

### Biophysical and Pharmacological Studies

### Example A: Binding Affinity Assays

### Method A: Inhibition of Mcl-1 by the Fluorescence Polarisation Assay

The relative binding potency of each compound was determined via Fluorescence Polarisation (FP). The method utilised a Fluorescein labelled ligand (Fluorescein-βAla-Ahx-A-REIGAQLRRMADDLNAQY-OH; MW 2,765) which binds to the Mcl-1 protein (such that Mcl-1 corresponds to the UniProtKB® primary accession number: Q07820) leading to an increased anisotropy measured in milli-polarisation (mP) units using a reader. The addition of a compound which binds competitively to the same site as the ligand will result in a greater proportion of unbound ligand in the system indicated by a decrease in mP units.

An 11 point serial dilution of each compound was prepared in DMSO and 2 μl transferred into flat bottomed, low binding, 384-well plate (final DMSO concentration 5%). 38 μl of buffer containing the Fluorescein labelled ligand (final concentration 1 nM) and Mcl-1 protein (final concentration 5 nM) was then added. Buffer components were 10 mM 4-(2-hydroxyethyl)-1-piperazineethanesulfonic acid [HEPES], 150 mM NaCl, pH 7.4 with the addition of 0.05% Tween 20.

Assay plates were incubated for 2 hours at room temperature before FP was measured on a Biomek Synergy Neo reader (Ex. 528 nm, Em. 640 nm, Cut off 510 nm) and mP units calculated. The binding of increasing doses of test compound was expressed as a percentage reduction in mP compared to a window established between ‘5% DMSO only’ and ‘100% inhibition’ controls. 11-point dose response curves were plotted with XL-Fit software using a 4-Parameter Logistic Model (Sigmoidal Dose-Response Model) and the inhibitory concentrations that gave a 50% reduction in mP (IC₅₀) were determined. The Ki values were determined from the IC₅₀ values according to Cer et al, Nucleic Acids Res, 2009, 37 (WebServer issue): W441-W445. Inhibition constants (Ki) are determined from complete binding inhibition curves (cKi) or from estimated from incomplete binding inhibition curves (eKi) in most cases due to low activity.

### Method B: Inhibition of Mcl-1 by the Fluorescence Quenching Assay

Fluorescence quenching assay measures the change fluorescence intensity of C-terminally Cy5-labelled Mcl1 (amino acids 171-321) C286S protein (UniProtKB® primary accession number Q07820) having an amino acid sequence:

[MHHHHHHSSGLVPRGSGMKETAAAKFERQHMDSPDLGTDDDDKAMAHH
HHHHSSENLYFQGPLGSEDELYRQSLEIISRYLREQATGAKDTKPMGRS
GATSRKALETLRRVGDGVQRNHETAFQGMLRKLDIKNEDDVKSLSRVMI
HVFSDGVTNWGRIVTLISFGAFVAKHLKTINQESSIEPLAESITDVLVR
TKRDWLVKQRGWDGFVEFFH]

which is linked at the C-terminus to the amino acid X which corresponds to a cysteine labelled on the sulfur with sulpho-Cyanine5 from Lumiprobe GmbH catalogue number 13380,
upon binding of a C-terminally labelled peptide derived from PUMA (UniProtKB® primary accession number Q9BXH1) having an amino acid sequence:

[QWAREIGAQLRRMADDLNAQY]

which is linked at the C-terminus to the amino acid X′, where X′ is cysteine labelled on the sulfur with TQ5WS from AAT Bioquest catalogue number 2079.

The addition of a compound which binds competitively to the same site as the peptide will result in an increase in the fluorescence intensity of the protein due to displacement of the fluorescence quencher.

An 11-point serial dilution of each compound was prepared in DMSO, the final buffer conditions were 10 mM 4-(2-hydroxyethyl)-1-piperazineethanesulfonic acid [HEPES], 150 mM NaCl, 0.05% Tween 20, pH 7.4 and 5% DMSO. The final protein concentration in the assay was 1 nM with the peptide present at 10, 20 or 400 nM. The experiments were incubated for 2 hours at room temperature before fluorescence intensity was measured on a Biotek SynergyNeo plate reader (Excitation 620 nm, emission 680 nm). The dose response curves were plotted with XL-Fit software using a 4-Parameter Logistic Model (Sigmoidal DoseResponse Model) and the inhibitory concentrations that gave a 50% increase in fluorescence intensity was determined (IC₅₀). The Ki values were determined from the IC₅₀ values according to Cer et al, Nucleic Acids Res, 2009, 37 (WebServer issue): W441-W445. Inhibition constants (Ki) are determined from complete binding inhibition curves (cKi) or from estimated from incomplete binding inhibition curves (eKi) in most cases due to low activity.

The results are summarized in Table 1 below and show that the compounds of the invention inhibit interaction between the Mcl-1 protein and the fluorescent peptide described hereinbefore.

TABLE 1
Determination of Mcl-1 inhibition by fluorescence polarization assay
(Method A) and by fluorescence quenching assay (Method B)

| Ex. | Method | Ki (M) |
|---|---|---|
| 1 | A | 2.33E−05 |
| 2 | A | 7.67E−06 |
| 3 | A | 1.28E−05 |
| 4 | A | 5.95E−06 |
| 5 | A | 2.20E−05 |
| 6 | A | 9.91E−06 |
| 7 | A | 2.38E−05 |
| 8 | A | 2.25E−05 |
| 9 | A | 4.82E−06 |
| 10 | A | 1.07E−05 |
| 11 | A | 6.43E−06 |
| 12 | A | 1.24E−05 |
| 13 | A | 7.08E−06 |
| 14 | A | 5.20E−06 |
| 15 | A | 1.15E−05 |
| 17 | A | 2.31E−05 |
| 18 | A | 3.19E−05 |
| 19 | A | 2.11E−05 |
| 20 | A | 2.57E−05 |
| 22 | A | 2.41E−05 |
| 23 | A | 1.08E−06 |
| 24 | A | 1.99E−05 |
| 25 | A | 8.87E−07 |
| 26 | A | 4.28E−05 |
| 27 | A | 3.63E−05 |
| 28 | A | 1.48E−05 |
| 29 | A | 1.58E−05 |
| 30 | A | 9.25E−06 |
| 31 | A | 3.63E−05 |
| 32 | A | 3.21E−06 |
| 33 | A | 1.21E−05 |
| 34 | A | 6.48E−07 |
| 35 | A | 3.32E−07 |
| 36 | B | 7.49E−07 |
| 37 | A | 2.95E−07 |
| 38 | B | 1.44E−08 |
| 39 | B | 4.49E−08 |
| 40 | B | 9.81E−10 |
| 41 | B | 3.55E−10 |
| 42 | B | 2.94E−10 |
| 43 | B | 2.50E−10 |
| 44 | B | 1.26E−10 |
| 45 | B | 3.00E−07 |
| 46 | B | 2.55E−10 |
| 47 | B | 9.33E−11 |
| 48 | B | 1.04E−07 |
| 49 | B | 3.10E−10 |
| 50 | B | 7.20E−08 |
| 51 | B | 7.62E−11 |
| 52 | B | 5.83E−10 |
| 53 | B | 1.50E−09 |
| 54 | B | 1.77E−10 |
| 55 | B | 1.04E−10 |
| 56 | B | 7.62E−10 |
| 57 | A | 9.34E−07 |
| 58 | A | 6.96E−07 |
| 59 | A | 4.21E−06 |
| 60 | A | 2.76E−07 |
| 61 | A | 3.58E−07 |
| 62 | A | 4.35E−05 |
| 63 | A | 2.40E−07 |
| 64 | A | 7.35E−07 |
| 65 | A | 4.99E−06 |
| 67 | A | 1.01E−07 |
| 68 | A | 3.02E−07 |
| 69 | A | 7.06E−07 |
| 70 | A | 2.59E−05 |
| 71 | A | 9.84E−07 |
| 72 | A | 1.91E−04 |
| 73 | A | 6.05E−05 |
| 74 | A | 2.31E−05 |
| 75 | B | 1.19E−07 |
| 76 | A | 2.84E−07 |
| 77 | A | 2.27E−07 |
| 78 | A | 8.76E−08 |
| 79 | A | 5.71E−07 |
| 80 | A | 1.18E−05 |
| 81 | A | 1.55E−05 |
| 82 | B | 4.37E−07 |
| 83 | A | 1.36E−06 |
| 84 | A | 7.22E−06 |
| 85 | B | 4.24E−07 |
| 86 | A | 4.10E−06 |
| 87 | A | 4.38E−06 |
| 88 | B | 5.03E−07 |
| 89 | A | 2.12E−06 |
| 90 | A | 2.15E−06 |
| 91 | A | 1.43E−06 |
| 92 | A | 7.37E−06 |
| 93 | A | 7.31E−06 |
| 94 | A | 1.29E−04 |
| 95 | A | 1.19E−06 |
| 96 | A | 4.50E−06 |
| 97 | A | 5.78E−07 |
| 98 | A | 1.04E−05 |
| 99 | A | 2.86E−06 |
| 100 | A | 2.62E−06 |
| 101 | A | 2.70E−05 |
| 102 | A | 1.07E−05 |
| 103 | A | 2.14E−07 |
| 104 | A | 1.36E−06 |
| 105 | A | 3.70E−06 |
| 106 | A | 2.26E−06 |
| 107 | A | 1.87E−06 |
| 108 | A | 5.73E−06 |
| 109 | A | 7.45E−07 |
| 110 | A | 8.45E−06 |
| 111 | A | 9.68E−06 |
| 112 | A | 1.62E−06 |
| 113 | A | 2.23E−05 |
| 114 | A | 2.73E−05 |
| 115 | A | 1.14E−06 |
| 116 | A | 8.82E−07 |
| 117 | A | 5.76E−06 |
| 118 | A | 3.99E−06 |
| 119 | A | 8.36E−07 |
| 120 | A | 1.93E−05 |
| 121 | A | 1.55E−06 |
| 122 | A | 3.77E−06 |
| 123 | A | 6.40E−06 |
| 124 | A | 1.29E−06 |
| 125 | A | 7.50E−06 |
| 126 | A | 9.15E−06 |
| 127 | A | 2.82E−06 |
| 128 | A | 4.62E−05 |
| 129 | A | 8.22E−08 |
| 130 | A | 9.24E−08 |
| 132 | A | 1.50E−07 |
| 133 | A | 5.76E−08 |
| 134 | A | 2.65E−07 |
| 135 | A | 2.00E−07 |
| 136 | A | 2.59E−07 |
| 137 | A | 1.92E−07 |
| 138 | A | 2.19E−07 |
| 139 | A | 2.65E−07 |
| 140 | B | 5.74E−08 |
| 141 | B | 9.16E−08 |
| 142 | A | 2.61E−07 |
| 143 | A | 1.82E−07 |
| 144 | A | 1.82E−07 |
| 145 | A | 4.14E−07 |
| 146 | A | 6.92E−06 |
| 147 | A | 8.16E−07 |
| 148 | A | 2.58E−07 |
| 149 | A | 6.33E−05 |
| 150 | A | 2.26E−07 |
| 151 | A | 1.34E−06 |
| 152 | A | 6.09E−07 |
| 153 | A | 5.07E−07 |
| 154 | A | 1.62E−07 |
| 155 | A | 7.10E−07 |
| 156 | A | 6.21E−07 |
| 157 | A | 8.46E−07 |
| 158 | A | 3.11E−07 |
| 159 | A | 2.33E−07 |
| 160 | A | 7.68E−07 |
| 161 | A | 4.47E−07 |
| 162 | A | 5.58E−07 |
| 163 | A | 6.79E−07 |
| 164 | A | 6.00E−07 |
| 165 | A | 9.04E−05 |
| 166 | A | 6.88E−07 |
| 167 | A | 3.27E−05 |
| 168 | A | 1.67E−06 |
| 169 | A | 4.77E−06 |
| 170 | A | 1.92E−06 |
| 171 | A | 5.14E−08 |
| 172 | A | 5.58E−08 |
| 173 | A | 4.55E−07 |
| 174 | A | 1.38E−07 |
| 175 | A | 1.54E−06 |
| 176 | A | 1.18E−05 |
| 177 | A | 2.26E−07 |
| 178 | B | 3.71E−08 |
| 179 | A | 5.06E−07 |
| 180 | A | 1.28E−05 |
| 181 | A | 1.14E−05 |
| 182 | A | 1.62E−06 |
| 183 | A | 2.48E−06 |
| 184 | A | 1.23E−06 |
| 185 | A | 7.40E−08 |
| 186 | A | 1.23E−07 |
| 187 | A | 2.71E−07 |
| 188 | A | 2.14E−07 |
| 189 | A | 1.21E−07 |
| 190 | A | 4.43E−08 |
| 191 | A | 6.97E−08 |
| 192 | A | 6.15E−08 |
| 193 | A | 5.31E−07 |
| 195 | A | 2.66E−07 |
| 196 | A | 9.47E−06 |
| 197 | A | 1.38E−06 |
| 198 | A | 7.48E−06 |
| 199 | B | 1.25E−06 |
| 200 | B | 1.39E−07 |
| 201 | A | 1.53E−06 |
| 202 | A | 3.00E−07 |
| 203 | A | 4.01E−05 |
| 204 | A | 2.24E−05 |
| 205 | A | 1.71E−06 |
| 206 | A | 4.68E−06 |
| 207 | A | 4.66E−06 |
| 208 | A | 3.16E−05 |
| 209 | A | 3.50E−05 |
| 210 | A | 8.90E−05 |
| 211 | A | 1.10E−05 |
| 212 | A | 9.47E−06 |
| 213 | A | 8.73E−05 |
| 214 | A | 7.90E−06 |
| 215 | A | 8.46E−07 |
| 216 | A | 1.74E−06 |
| 217 | A | 4.84E−07 |
| 218 | A | 1.34E−05 |
| 219 | A | 3.81E−07 |
| 220 | A | 7.23E−06 |
| 221 | A | 9.95E−07 |
| 222 | A | 2.26E−06 |
| 223 | A | 1.43E−05 |
| 224 | A | 2.23E−05 |
| 225 | A | 1.02E−06 |
| 226 | A | 2.37E−05 |
| 227 | A | 1.19E−06 |
| 228 | A | 1.47E−05 |
| 229 | A | 1.34E−06 |
| 230 | A | 1.06E−06 |
| 231 | A | 7.72E−06 |
| 232 | A | 5.78E−07 |
| 233 | A | 4.40E−05 |
| 234 | A | 1.15E−04 |
| 235 | A | 1.36E−04 |
| 236 | A | 2.71E−05 |
| 237 | A | 8.90E−05 |
| 238 | A | 5.78E−05 |
| 239 | A | 3.01E−07 |
| 240 | A | 2.17E−07 |
| 241 | A | 4.35E−06 |
| 242 | A | 1.21E−06 |
| 243 | A | 2.73E−07 |
| 244 | A | 7.79E−07 |
| 245 | A | 4.30E−06 |
| 246 | A | 4.20E−06 |
| 247 | A | 1.22E−06 |
| 248 | A | 2.82E−05 |
| 249 | A | 1.90E−05 |
| 250 | A | 1.39E−05 |
| 251 | A | 1.69E−06 |
| 252 | A | 5.27E−06 |
| 253 | A | 8.36E−07 |
| 254 | A | 2.17E−06 |
| 255 | A | 1.16E−05 |
| 256 | A | 2.42E−05 |
| 257 | A | 4.04E−05 |
| 258 | A | 2.37E−06 |
| 259 | A | 9.97E−06 |
| 260 | A | 9.01E−05 |
| 261 | A | 9.40E−06 |
| 262 | A | 2.36E−07 |
| 263 | A | 5.02E−06 |
| 264 | A | 1.75E−06 |
| 265 | A | 1.10E−06 |
| 266 | A | 5.93E−07 |
| 267 | A | 2.59E−07 |
| 268 | A | 1.02E−06 |
| 269 | A | 1.11E−05 |
| 270 | A | 3.96E−06 |
| 271 | A | 1.25E−05 |
| 272 | A | 1.27E−07 |
| 273 | A | 1.72E−06 |
| 274 | A | 4.69E−06 |
| 275 | A | 1.01E−05 |
| 276 | A | 3.67E−05 |
| 277 | A | 6.09E−06 |
| 278 | A | 1.18E−05 |
| 279 | A | 3.83E−07 |
| 280 | A | 9.99E−06 |
| 281 | A | 4.73E−07 |
| 282 | A | 5.13E−06 |
| 283 | A | 4.16E−06 |
| 284 | A | 1.22E−06 |
| 285 | A | 4.96E−06 |
| 286 | A | 5.80E−06 |
| 287 | A | 9.25E−07 |
| 288 | A | 1.22E−05 |
| 289 | A | 5.69E−07 |
| 290 | A | 1.44E−05 |
| 291 | A | 2.54E−06 |
| 292 | A | 2.14E−05 |
| 293 | A | 4.99E−07 |
| 294 | A | 6.63E−06 |
| 295 | A | 6.43E−07 |
| 296 | A | 3.46E−06 |
| 297 | A | 2.37E−06 |
| 298 | A | 1.11E−06 |
| 299 | A | 4.10E−07 |
| 300 | A | 1.74E−07 |
| 301 | A | 4.91E−07 |
| 302 | A | 1.51E−06 |
| 303 | A | 2.26E−06 |
| 304 | A | 1.74E−06 |
| 305 | A | 6.63E−07 |
| 306 | A | 6.63E−07 |
| 307 | A | 9.17E−06 |
| 308 | A | 2.93E−06 |
| 309 | A | 7.32E−07 |
| 310 | A | 7.21E−07 |
| 311 | A | 5.59E−07 |
| 312 | A | 8.39E−07 |
| 313 | A | 3.85E−07 |
| 314 | A | 5.28E−07 |
| 315 | A | 5.46E−07 |
| 316 | A | 1.87E−06 |
| 317 | A | 9.42E−07 |
| 318 | A | 1.91E−06 |
| 319 | A | 9.06E−07 |
| 320 | A | 6.86E−06 |
| 321 | A | 2.94E−06 |
| 322 | A | 1.77E−05 |
| 323 | A | 1.06E−07 |
| 324 | A | 8.45E−06 |
| 325 | A | 2.89E−07 |
| 326 | A | 1.93E−05 |
| 327 | A | 7.14E−06 |
| 328 | A | 6.77E−07 |
| 329 | A | 1.65E−06 |
| 330 | A | 2.12E−06 |
| 331 | A | 2.18E−06 |
| 332 | A | 4.75E−07 |
| 333 | A | 1.32E−05 |
| 334 | A | 2.00E−06 |
| 335 | A | 8.89E−07 |
| 336 | A | 2.44E−06 |
| 337 | A | 2.47E−07 |
| 338 | A | 5.69E−06 |
| 339 | A | 1.54E−07 |
| 340 | A | 9.21E−07 |
| 341 | A | 1.59E−07 |
| 342 | A | 7.85E−07 |
| 343 | A | 9.78E−07 |
| 344 | A | 1.12E−05 |
| 345 | A | 1.24E−06 |
| 346 | A | 9.29E−06 |
| 347 | A | 1.13E−06 |
| 348 | A | 1.06E−05 |
| 349 | A | 3.59E−06 |
| 350 | A | 1.75E−06 |
| 351 | A | 5.44E−07 |
| 352 | A | 8.02E−07 |
| 353 | A | 8.72E−06 |
| 354 | A | 4.02E−07 |
| 355 | A | 9.96E−06 |
| 356 | A | 4.76E−07 |
| 357 | A | 1.54E−05 |
| 358 | A | 6.22E−08 |
| 359 | A | 2.14E−06 |
| 360 | A | 1.75E−05 |
| 361 | A | 1.48E−05 |
| 362 | A | 1.74E−07 |
| 363 | A | 2.27E−07 |
| 364 | A | 4.59E−07 |
| 365 | A | 3.09E−06 |
| 366 | A | 1.39E−06 |
| 367 | A | 4.43E−07 |
| 368 | A | 1.32E−06 |
| 369 | A | 1.91E−06 |
| 372 | A | 2.01E−07 |
| 373 | A | 2.21E−07 |
| 374 | A | 1.89E−07 |
| 375 | A | 4.79E−06 |
| 376 | A | 2.67E−07 |
| 377 | A | 7.43E−08 |
| 378 | A | 6.32E−08 |
| 379 | A | 3.10E−06 |
| 384 | A | 5.88E−08 |
| 385 | A | 8.10E−07 |
| 386 | A | 1.61E−07 |
| 387 | A | 2.20E−07 |
| 393 | A | 1.50E−07 |
| 394 | A | 1.34E−07 |
| 395 | A | 1.01E−07 |
| 396 | A | 3.21E−07 |
| 397 | A | 5.98E−07 |
| 398 | A | 1.31E−06 |
| 399 | A | 2.99E−07 |
| 400 | A | 9.17E−07 |
| 401 | A | 3.20E−07 |
| 402 | A | 1.88E−05 |
| 403 | A | 8.21E−06 |
| 404 | A | 7.78E−06 |
| 405 | A | 1.52E−05 |
| 406 | A | 6.17E−06 |
| 407 | A | 1.37E−06 |
| 408 | A | 4.27E−06 |
| 409 | A | 1.55E−07 |
| 410 | A | 5.50E−07 |
| 411 | A | 5.83E−07 |
| 412 | A | 4.73E−07 |
| 413 | A | 7.04E−07 |
| 414 | A | 3.12E−07 |
| 415 | A | 3.65E−07 |
| 416 | A | 6.23E−07 |
| 417 | A | 5.22E−07 |
| 418 | A | 6.48E−07 |
| 419 | A | 3.00E−07 |
| 420 | A | 1.10E−06 |
| 421 | A | 7.93E−07 |
| 422 | A | 9.59E−08 |
| 423 | A | 2.24E−07 |
| 424 | A | 1.84E−07 |
| 425 | A | 4.77E−07 |
| 426 | A | 5.53E−08 |
| 427 | A | 2.26E−07 |
| 428 | A | 3.93E−07 |
| 429 | A | 1.65E−07 |
| 430 | A | 2.06E−07 |
| 431 | A | 1.38E−07 |
| 432 | A | 3.89E−08 |
| 433 | A | 1.75E−06 |
| 434 | A | 9.35E−08 |
| 435 | A | 5.55E−07 |
| 436 | A | 9.18E−07 |
| 437 | A | 7.76E−08 |
| 438 | A | 2.02E−07 |
| 439 | A | 6.86E−08 |
| 440 | A | 4.70E−08 |
| 441 | A | 6.91E−08 |
| 442 | A | 1.07E−07 |
| 443 | A | 1.06E−07 |
| 444 | A | 8.52E−08 |
| 445 | A | 7.53E−08 |
| 446 | A | 1.34E−07 |
| 447 | A | 1.27E−07 |
| 448 | A | 6.54E−08 |
| 449 | A | 8.00E−08 |
| 450 | A | 7.81E−07 |
| 451 | A | 4.97E−08 |
| 452 | A | 3.00E−08 |
| 453 | A | 3.18E−07 |
| 454 | A | 1.41E−07 |
| 455 | A | 1.03E−06 |
| 456 | A | 1.04E−07 |
| 457 | A | 1.29E−07 |
| 458 | A | 1.10E−07 |
| 459 | A | 8.57E−08 |
| 460 | A | 1.59E−07 |
| 461 | A | 3.25E−07 |
| 463 | A | 5.92E−08 |
| 464 | A | 4.91E−08 |
| 465 | A | 7.54E−08 |
| 466 | A | 5.66E−08 |
| 467 | A | 7.46E−07 |
| 468 | A | 2.34E−07 |
| 469 | A | 3.24E−07 |
| 470 | A | 1.84E−07 |
| 471 | A | 6.20E−07 |
| 472 | A | 3.30E−08 |
| 473 | A | 8.19E−08 |
| 474 | A | 9.60E−08 |
| 475 | A | 9.92E−08 |
| 476 | A | 1.69E−07 |
| 477 | A | 2.36E−07 |
| 478 | A | 2.97E−08 |
| 479 | A | 8.09E−07 |
| 480 | A | 4.31E−07 |
| 481 | A | 7.21E−08 |
| 482 | A | 3.17E−07 |
| 483 | A | 1.74E−07 |
| 484 | A | 7.59E−07 |
| 485 | B | 4.55E−08 |
| 486 | B | 8.20E−08 |
| 487 | B | 7.29E−08 |
| 488 | B | 1.52E−07 |
| 489 | B | 7.60E−08 |
| 490 | B | 6.18E−08 |
| 491 | A | 1.54E−07 |
| 492 | A | 9.37E−08 |
| 493 | A | 1.78E−06 |
| 494 | B | 3.62E−09 |
| 495 | A | 1.14E−08 |
| 496 | B | 1.80E−09 |
| 497 | B | 1.34E−09 |
| 498 | B | 7.69E−10 |
| 499 | B | 7.41E−08 |
| 500 | B | 1.55E−08 |
| 501 | B | 4.80E−09 |
| 502 | A | 7.78E−05 |
| 503 | B | 6.38E−08 |
| 504 | B | 2.99E−08 |
| 505 | B | 2.34E−08 |
| 506 | B | 4.92E−08 |
| 507 | B | 8.76E−09 |
| 508 | B | 2.03E−08 |
| 509 | B | 5.30E−09 |
| 510 | B | 1.51E−09 |
| 511 | B | 1.99E−08 |
| 512 | B | 2.27E−08 |
| 513 | B | 2.79E−08 |
| 514 | B | 1.84E−09 |
| 515 | B | 2.64E−08 |
| 516 | B | 2.80E−08 |
| 517 | B | 4.98E−09 |
| 518 | B | 1.26E−06 |
| 519 | B | 9.09E−10 |
| 520 | B | 9.14E−08 |
| 521 | B | 3.23E−08 |
| 522 | A | 6.36E−06 |
| 523 | A | 7.63E−06 |
| 524 | A | 1.68E−06 |
| 525 | A | 2.93E−07 |
| 526 | A | 1.86E−06 |
| 527 | A | 8.94E−06 |
| 528 | A | 3.28E−06 |
| 529 | A | 4.31E−06 |
| 530 | A | 2.05E−06 |
| 531 | A | 5.97E−06 |
| 532 | A | 8.09E−07 |
| 533 | A | 7.85E−07 |
| 534 | A | 1.52E−06 |
| 535 | A | 2.28E−06 |
| 536 | A | 2.10E−06 |
| 537 | A | 5.75E−07 |
| 538 | B | 9.15E−09 |
| 539 | B | 2.54E−08 |
| 540 | A | 1.85E−07 |
| 541 | A | 1.17E−06 |
| 542 | A | 1.87E−07 |
| 543 | B | 1.26E−07 |
| 544 | B | 2.24E−08 |
| 545 | A | 3.49E−06 |
| 546 | A | 1.64E−06 |
| 547 | B | 6.95E−09 |
| 548 | A | 9.95E−07 |
| 549 | A | 9.48E−06 |
| 550 | A | 5.28E−06 |
| 551 | A | 6.07E−07 |
| 552 | A | 1.10E−07 |
| 553 | A | 5.64E−06 |
| 554 | A | 3.23E−07 |
| 555 | B | 1.46E−08 |
| 556 | A | 7.19E−06 |
| 557 | A | 1.27E−06 |
| 558 | B | 5.49E−07 |
| 559 | B | 7.30E−08 |
| 560 | B | 9.20E−08 |
| 561 | B | 5.71E−08 |
| 562 | B | 8.09E−08 |
| 563 | B | 4.35E−08 |
| 564 | B | 2.99E−08 |
| 565 | B | 1.08E−08 |
| 566 | B | 4.34E−09 |
| 567 | A | 1.55E−07 |
| 568 | A | 4.38E−07 |
| 569 | A | 7.43E−08 |
| 570 | B | 3.63E−07 |
| 571 | B | 1.07E−07 |
| 572 | B | 8.42E−08 |
| 573 | B | 8.06E−07 |
| 574 | B | 2.21E−07 |
| 575 | B | 3.50E−07 |
| 576 | A | 1.24E−07 |
| 577 | B | 1.04E−08 |
| 578 | B | 7.84E−09 |
| 579 | A | 2.16E−07 |
| 580 | A | 9.27E−08 |
| 581 | A | 4.75E−08 |
| 582 | A | 5.19E−08 |
| 583 | A | 1.91E−07 |
| 584 | B | 7.03E−09 |
| 585 | A | 3.60E−08 |
| 586 | B | 2.62E−09 |
| 587 | B | 3.93E−09 |
| 588 | B | 2.68E−09 |
| 589 | B | 8.39E−09 |
| 590 | A | 1.92E−08 |
| 591 | B | 2.72E−09 |
| 592 | A | 7.10E−07 |
| 593 | A | 1.45E−08 |
| 594 | B | 4.41E−08 |
| 595 | A | 2.99E−08 |
| 596 | A | 3.12E−08 |
| 597 | B | 5.36E−09 |
| 598 | B | 4.29E−09 |
| 599 | B | 1.05E−09 |
| 600 | A | 4.26E−09 |
| 601 | A | 3.28E−08 |
| 602 | A | 3.37E−09 |
| 603 | B | 8.00E−09 |
| 604 | A | 3.00E−08 |
| 605 | A | 2.42E−08 |
| 606 | A | 7.73E−08 |
| 607 | A | 1.64E−07 |
| 608 | B | 1.62E−09 |
| 609 | B | 2.61E−09 |
| 610 | B | 7.67E−10 |
| 611 | B | 1.23E−08 |
| 612 | B | 5.65E−10 |
| 613 | B | 1.89E−08 |
| 614 | B | 1.23E−07 |
| 615 | B | 2.41E−07 |
| 616 | B | 1.21E−08 |
| 617 | B | 9.35E−08 |
| 618 | B | 4.26E−08 |
| 619 | B | 9.28E−09 |
| 620 | B | 1.27E−08 |
| 621 | B | 3.76E−08 |
| 622 | B | 4.46E−08 |
| 623 | B | 2.32E−06 |
| 624 | B | 4.48E−09 |
| 625 | B | 3.77E−08 |
| 626 | B | 3.91E−09 |
| 627 | B | 7.36E−09 |
| 628 | B | 6.99E−08 |
| 629 | B | 9.55E−09 |
| 630 | B | 1.17E−09 |
| 631 | B | 1.26E−08 |
| 632 | B | 6.01E−09 |
| 633 | A | 5.30E−09 |
| 634 | A | 8.68E−08 |
| 635 | A | 4.48E−08 |
| 636 | B | 1.62E−08 |
| 637 | B | 5.13E−09 |
| 638 | B | 3.76E−09 |
| 639 | B | 3.72E−08 |
| 640 | B | 9.80E−07 |
| 641 | B | 1.40E−08 |
| 642 | B | 7.24E−10 |
| 643 | B | 2.68E−08 |
| 644 | B | 2.80E−09 |
| 645 | B | 4.04E−09 |
| 646 | B | 5.93E−10 |
| 647 | B | 9.39E−09 |
| 648 | B | 1.80E−08 |
| 649 | B | 6.36E−09 |
| 650 | B | 5.54E−09 |
| 651 | A | 1.79E−08 |
| 652 | A | 2.03E−08 |
| 653 | B | 2.82E−10 |
| 654 | B | 5.20E−09 |
| 655 | B | 5.09E−08 |
| 656 | B | 6.29E−08 |
| 657 | B | 2.94E−09 |
| 658 | B | 3.42E−08 |
| 659 | B | 5.73E−09 |
| 660 | B | 1.39E−09 |
| 661 | B | 8.07E−08 |
| 662 | B | 6.87E−08 |
| 663 | B | 2.31E−08 |
| 664 | B | 5.25E−08 |
| 665 | B | 3.01E−09 |
| 666 | B | 2.87E−08 |
| 667 | B | 4.05E−09 |
| 668 | B | 2.74E−09 |
| 669 | A | 3.23E−08 |
| 670 | B | 4.16E−08 |
| 671 | B | 3.93E−08 |
| 672 | B | 2.44E−09 |
| 673 | B | 6.47E−10 |
| 674 | B | 6.95E−09 |
| 675 | B | 2.99E−08 |
| 676 | B | 4.18E−10 |
| 677 | B | 1.18E−09 |
| 678 | B | 2.21E−07 |
| 679 | B | 2.32E−09 |
| 680 | B | 2.09E−09 |
| 681 | B | 3.26E−09 |
| 682 | B | 4.66E−08 |
| 683 | B | 1.06E−07 |
| 684 | B | 4.55E−08 |
| 685 | B | 5.80E−09 |
| 686 | B | 1.85E−09 |
| 687 | B | 4.25E−09 |
| 688 | B | 9.65E−10 |
| 689 | B | 1.46E−09 |
| 690 | B | 2.95E−08 |
| 691 | B | 1.09E−08 |
| 692 | B | 1.75E−09 |
| 693 | B | 3.70E−09 |
| 694 | B | 1.00E−07 |
| 695 | B | 2.96E−08 |
| 696 | B | 1.03E−07 |
| 697 | B | 8.28E−09 |
| 698 | B | 6.56E−08 |
| 699 | B | 1.15E−09 |
| 700 | B | 5.09E−09 |
| 701 | B | 2.85E−08 |
| 702 | B | 1.94E−10 |
| 703 | B | 1.27E−09 |
| 704 | B | 1.49E−09 |
| 705 | B | 2.77E−08 |
| 706 | B | 9.01E−10 |
| 707 | B | 5.85E−10 |
| 708 | B | 3.62E−10 |
| 709 | B | 6.40E−09 |
| 710 | B | 4.41E−08 |
| 711 | B | 4.46E−10 |
| 712 | B | 4.39E−10 |
| 713 | B | 3.23E−09 |
| 714 | B | 4.75E−09 |
| 715 | B | 2.01E−09 |
| 716 | B | 4.71E−09 |
| 717 | B | 2.28E−09 |
| 718 | B | 14.8% @ 10 μM |
| 719 | B | 1.45E−06 |
| 721 | B | 2.12E−09 |
| 722 | B | 3.49E−10 |
| 729 | B | 7.68E−09 |
| 730 | B | 2.10E−09 |
| 733 | B | 1.52E−09 |
| 734 | B | 1.98E−09 |
| 735 | B | 2.55E−09 |
| 736 | B | 5.19E−09 |
| 737 | B | 9.32E−10 |
| 738 | B | 7.40E−09 |
| 739 | B | 1.50E−09 |
| 740 | B | 8.27E−10 |
| 741 | B | 1.58E−09 |
| 742 | B | 1.23E−09 |
| 743 | B | 4.63E−10 |
| 744 | B | 3.42E−10 |
| 745 | B | 1.58E−09 |
| 746 | B | 3.56E−10 |
| 747 | B | 7.88E−10 |
| 748 | B | 1.50E−09 |
| 749 | B | 3.21E−10 |
| 750 | B | 1.06E−09 |
| 751 | B | 6.44E−10 |
| 752 | B | 3.21E−10 |
| 753 | B | 3.22E−09 |
| 754 | B | 1.23E−09 |
| 755 | B | 9.40E−10 |
| 756 | B | 1.01E−09 |
| 757 | B | 1.90E−09 |
| 758 | B | 1.19E−09 |
| 759 | B | 3.03E−10 |
| 760 | B | 1.98E−10 |
| 761 | B | 6.53E−08 |
| 762 | B | 1.21E−08 |
| 763 | B | 8.13E−11 |
| 765 | B | 6.61E−10 |
| 766 | B | 2.30E−09 |
| 767 | B | 3.05E−10 |
| 768 | B | 1.09E−10 |
| 769 | B | 1.52E−09 |
| 770 | B | 3.50E−10 |
| 771 | B | 1.76E−10 |
| 772 | B | 3.63E−09 |
| 773 | B | 2.79E−10 |
| 774 | B | 1.42E−09 |
| 775 | B | 5.18E−10 |
| 776 | B | 2.28E−09 |
| 777 | B | 7.25E−10 |
| 778 | B | 1.77E−10 |
| 779 | B | 4.23E−10 |
| 780 | B | 8.89E−09 |
| 781 | B | 2.34E−10 |
| 782 | B | 2.54E−10 |
| 783 | B | 1.03E−08 |
| 784 | B | 8.01E−11 |
| 785 | B | 1.22E−10 |
| 786 | B | 8.03E−11 |
| 787 | B | 5.73E−11 |
| 788 | B | 2.41E−10 |
| 789 | B | 1.35E−10 |
| 790 | B | 7.64E−10 |
| 791 | B | 1.24E−10 |
| 792 | B | 4.69E−10 |
| 793 | B | 1.79E−08 |
| 794 | B | 2.13E−10 |
| 797 | B | 2.59E−10 |
| 813 | B | 9.08E−11 |
| 814 | B | 3.42E−09 |
| 815 | B | 2.51E−10 |
| 816 | B | 3.53E−10 |
| 817 | B | 2.29E−10 |
| 818 | B | 7.83E−11 |
| 819 | B | 1.62E−10 |
| 820 | B | 4.69E−10 |
| 821 | B | 4.08E−10 |
| 822 | B | 1.49E−10 |
| 823 | B | 1.08E−10 |
| 824 | B | 3.29E−10 |
| 835 | B | 4.38E−10 |
| 836 | B | 4.72E−11 |
| 837 | B | 7.33E−12 |
| 838 | B | 3.37E−11 |
| 839 | B | 11.1% @ 1 μM |
| 840 | B | 5.54E−11 |
| 841 | B | 1.72E−10 |
| 842 | B | 1.55E−09 |
| 843 | B | 3.58E−09 |
| 844 | B | 1.06E−11 |
| 845 | B | 7.36E−11 |
| 846 | B | 1.05E−10 |
| 847 | B | 8.47E−11 |
| 848 | B | 3.43E−11 |
| 849 | B | 2.28E−10 |
| 850 | B | 1.73E−11 |
| 851 | B | 2.56E−11 |
| 852 | B | 8.96E−11 |
| 853 | B | 9.17E−11 |
| 854 | B | 4.55E−10 |
| 855 | B | 7.03E−10 |
| 856 | B | 8.64E−10 |
| 857 | B | 1.69E−10 |
| 858 | B | 6.99E−11 |
| 859 | B | 3.75E−11 |
| 860 | B | 1.88E−11 |
| 861 | B | 9.09E−11 |
| 862 | B | 1.13E−10 |
| 863 | B | 3.82E−10 |
| 864 | B | 3.74E−10 |
| 865 | B | 2.04E−10 |
| 866 | B | 2.13E−11 |
| 867 | B | 9.88E−11 |
| 868 | B | 1.90E−10 |
| 869 | B | 3.84E−10 |
| 870 | B | 5.09E−11 |
| 871 | B | 7.37E−10 |
| 872 | B | 5.05E−11 |
| 873 | B | 4.17E−09 |
| 874 | B | 1.61E−10 |
| 875 | B | 8.43E−11 |
| 876 | B | 3.99E−10 |
| 877 | B | 6.42E−10 |
| 878 | B | 3.86E−10 |
| 879 | B | 4.06E−09 |
| 880 | B | 1.23E−09 |
| 881 | B | 1.54E−10 |
| 882 | B | 1.84E−09 |
| 883 | B | 2.46E−10 |
| 885 | B | 2.03E−09 |
| 886 | B | 1.45E−10 |
| 887 | B | 2.93E−10 |
| 888 | B | 2.50E−10 |
| 919 | B | 2.67E−11 |
| 920 | B | 3.85E−09 |
| 921 | B | 1.08E−11 |
| 930 | B | 4.5% @ 1 μM |
| 931 | B | 9.5% @ 10 μM |
| 932 | B | 2.13E−07 |
| 933 | B | 3.03E−07 |
| 934 | B | 6.90E−08 |
| 935 | B | 5.46E−09 |
| 936 | B | 1.42E−07 |
| 937 | B | 2.80E−08 |
| 938 | B | 2.43E−09 |
| 939 | B | 1.70E−09 |
| 940 | B | 2.52E−09 |
| 941 | B | 3.94E−09 |
| 942 | B | 1.55E−08 |
| 943 | B | 1.00E−09 |
| 944 | B | 13.9% @ 1 μM |
| 945 | B | 1.62E−07 |
| 946 | B | 1.10E−08 |
| 947 | B | 2.56E−10 |
| 948 | B | 6.49E−08 |
| 949 | B | 13.6% @ 1 μM |
| 950 | B | 7.1% @ 1 μM |
| 951 | B | 3.69E−08 |
| 952 | B | 3.71E−10 |
| 953 | B | 5.56E−08 |
| 957 | B | 6.24E−09 |
| 959 | B | 1.17E−11 |
| 960 | B | 8.34E−11 |
| 961 | B | 3.08E−11 |
| 962 | B | 1.09E−11 |
| 963 | B | 2.35E−11 |
| 964 | B | 5.79E−11 |
| 965 | B | 1.73E−11 |
| 966 | B | 2.67E−11 |
| 967 | B | 2.63E−11 |
| 968 | B | 4.05E−11 |
| 971 | B | 1.43E−11 |
| 972 | B | 8.93E−12 |
| 973 | B | 1.18E−11 |
| 974 | B | 2.30E−11 |
| 975 | B | 2.44E−11 |
| 976 | B | 1.63E−11 |
| 977 | B | 2.31E−11 |
| 978 | B | 8.31E−11 |
| 979 | B | 2.99E−11 |
| 980 | B | 1.02E−10 |
| 981 | B | 2.38E−11 |
| 982 | B | 2.64E−11 |
| 983 | B | 3.70E−11 |
| 984 | B | 7.28E−12 |
| 985 | B | 9.55E−11 |
| 986 | B | 3.42E−11 |
| 987 | B | 4.47E−12 |
| 988 | B | 3.23E−11 |
| 989 | B | 2.88E−11 |
| 990 | B | 5.75E−11 |
| 991 | B | 7.64E−11 |
| 992 | B | 7.95E−11 |
| 993 | B | 6.66E−11 |
| 994 | B | 4.42E−11 |
| 995 | B | 8.85E−11 |
| 996 | B | 2.80E−11 |
| 997 | B | 4.15E−09 |
| 998 | B | 1.06E−10 |

### Example B: In Vitro Cytotoxicity

### Method A: MTT on 11929 Cell Lines

The cytotoxicity studies were carried out on the H929 multiple myeloma tumor line.

The cells are distributed onto microplates and exposed to the test compounds for 48 hours. The cell viability is then quantified by a colorimetric assay, the Microculture Tetrazolium Assay (Carmichael et al, Cancer Res. 1987, 47, 939-942).

The results are expressed in C50 which means concentration corresponding to 5000 of effect/inhibition.

### Method B: CTG on NCI-H1929 Cell Lines

Cellular viability was monitored with the cell viability assay (Cell Titer-Glo®) on NCI-H929 cell lines. The CellTiter-Glo® (CTG) Luminescent Cell Viability Assay (from Promega) is a homogeneous method to determine the number of viable cells in culture based on quantitation of the ATP present, which signals the presence of metabolically active cells.

Concentration response of test compounds are performed with 10 mM stock solutions (DMSO), 11-points curve, in a 384-well compound plate. The assay plate is prepared by stamping 120 nL from compound dilution plate of all different concentrations (e.g. 10 mM to have final 30 μM assay test) into a cell culture microplate, using Echo acoustic liquid handling (Labcyte, Beckman). 40 μl of cell solution is added and cells are incubated with compounds for 48 h at 37° C., 5% CO₂. CTG was added to the cells (according to the manufacturer instructions) and luminescence intensity was recorded using the Pherastar plate reader (BMG).

This assay provides in vitro cellular efficacy of compounds of Formula (I) tested, measuring the C₅₀, defined as the concentration needed to reach 50% of the cellular viability.

The results are summarized in Table 2 below and show that the compounds of the invention are cytotoxic.

TABLE 2
Determination of in vitro cytotoxicity by MTT assay (Method A)
and by CTG assay (Method B)

| Ex. | Method | C50 (M) |
|---|---|---|
| 34 | A | 3.00E−05 |
| 35 | A | 3.00E−05 |
| 36 | A | 1.85E−05 |
| 40 | B | 2.80E−07 |
| 41 | B | 1.15E−07 |
| 42 | A | 3.39E−08 |
| 43 | B | 1.62E−07 |
| 44 | B | 5.34E−08 |
| 45 | B | 2.42E−05 |
| 46 | B | 2.62E−07 |
| 47 | B | 3.74E−08 |
| 48 | B | 2.15E−05 |
| 49 | B | 2.42E−07 |
| 50 | B | 1.35E−05 |
| 51 | B | 3.77E−08 |
| 52 | B | 4.54E−07 |
| 53 | B | 4.51E−07 |
| 54 | B | 1.77E−07 |
| 55 | B | 1.81E−07 |
| 56 | B | 1.51E−07 |
| 57 | A | 3.00E−05 |
| 60 | A | 3.00E−05 |
| 61 | A | 3.00E−05 |
| 75 | A | 3.00E−05 |
| 144 | A | 3.00E−05 |
| 145 | A | 3.00E−05 |
| 147 | A | 3.00E−05 |
| 172 | A | 1.87E−05 |
| 190 | A | 6.40E−06 |
| 191 | A | 5.72E−06 |
| 192 | A | 4.19E−06 |
| 323 | A | 1.05E−05 |
| 332 | A | 1.64E−05 |
| 337 | A | 3.00E−05 |
| 341 | A | 9.26E−06 |
| 345 | A | 3.00E−05 |
| 346 | A | 3.00E−05 |
| 358 | A | 7.43E−06 |
| 362 | A | 2.16E−05 |
| 363 | A | 3.00E−05 |
| 378 | A | 9.24E−06 |
| 384 | A | 1.14E−05 |
| 385 | A | 1.89E−05 |
| 386 | A | 7.92E−06 |
| 387 | A | 9.59E−06 |
| 393 | A | 1.20E−05 |
| 409 | A | 3.00E−05 |
| 426 | A | 1.65E−05 |
| 427 | A | 1.74E−05 |
| 439 | A | 3.88E−06 |
| 440 | A | 3.48E−06 |
| 442 | A | 5.79E−06 |
| 444 | A | 5.44E−06 |
| 445 | A | 5.35E−06 |
| 448 | A | 1.05E−05 |
| 449 | A | 9.27E−06 |
| 451 | A | 3.49E−06 |
| 452 | A | 2.44E−06 |
| 459 | A | 6.96E−06 |
| 463 | A | 8.23E−06 |
| 464 | A | 1.48E−05 |
| 465 | A | 9.60E−06 |
| 472 | A | 5.04E−06 |
| 481 | A | 5.08E−06 |
| 482 | A | 1.24E−05 |
| 483 | A | 4.17E−06 |
| 484 | A | 9.64E−06 |
| 494 | A | 8.08E−07 |
| 497 | A | 1.22E−06 |
| 498 | A | 4.36E−07 |
| 501 | A | 4.31E−07 |
| 505 | A | 2.38E−06 |
| 506 | A | 2.79E−06 |
| 507 | A | 2.24E−06 |
| 508 | A | 3.64E−06 |
| 509 | A | 9.99E−07 |
| 510 | A | 2.52E−07 |
| 511 | A | 2.72E−06 |
| 514 | A | 3.20E−07 |
| 515 | A | 2.39E−06 |
| 516 | A | 3.86E−06 |
| 517 | A | 5.50E−07 |
| 518 | A | 1.20E−05 |
| 519 | A | 2.02E−07 |
| 520 | A | 1.04E−05 |
| 521 | A | 9.86E−07 |
| 522 | A | 1.71E−05 |
| 523 | A | 1.64E−05 |
| 524 | A | 3.00E−05 |
| 525 | A | 2.49E−06 |
| 526 | A | 3.00E−05 |
| 527 | A | 1.64E−05 |
| 528 | A | 3.00E−05 |
| 529 | A | 2.53E−05 |
| 530 | A | 1.53E−05 |
| 531 | A | 1.59E−05 |
| 532 | A | 1.26E−05 |
| 533 | A | 1.45E−05 |
| 535 | A | 3.00E−05 |
| 536 | A | 1.72E−05 |
| 537 | A | 1.60E−05 |
| 544 | A | 8.32E−06 |
| 545 | A | 8.92E−06 |
| 546 | A | 3.00E−05 |
| 547 | A | 3.76E−07 |
| 548 | A | 3.00E−05 |
| 549 | A | 1.79E−05 |
| 550 | A | 3.00E−05 |
| 551 | A | 1.59E−05 |
| 552 | A | 3.38E−06 |
| 553 | A | 1.75E−05 |
| 554 | A | 3.00E−05 |
| 555 | A | 2.86E−06 |
| 556 | A | 1.93E−05 |
| 557 | A | 2.01E−05 |
| 559 | A | 3.00E−05 |
| 560 | A | 1.26E−05 |
| 561 | A | 1.23E−05 |
| 562 | A | 1.78E−05 |
| 563 | A | 3.95E−06 |
| 564 | A | 1.61E−05 |
| 565 | A | 2.39E−06 |
| 566 | A | 7.21E−07 |
| 568 | A | 1.83E−05 |
| 569 | A | 1.72E−06 |
| 570 | A | 3.00E−05 |
| 571 | A | 3.00E−05 |
| 572 | A | 3.00E−05 |
| 573 | A | 3.00E−05 |
| 575 | A | 1.62E−05 |
| 577 | A | 1.19E−06 |
| 578 | A | 4.71E−07 |
| 580 | A | 5.12E−06 |
| 582 | A | 2.55E−06 |
| 584 | A | 1.76E−06 |
| 585 | A | 2.61E−06 |
| 586 | A | 1.61E−06 |
| 587 | A | 6.79E−07 |
| 589 | A | 6.94E−07 |
| 590 | A | 1.17E−06 |
| 591 | A | 4.38E−07 |
| 593 | A | 2.01E−06 |
| 595 | A | 1.51E−06 |
| 596 | A | 1.82E−06 |
| 597 | A | 1.31E−06 |
| 598 | A | 5.90E−07 |
| 599 | A | 2.37E−07 |
| 600 | A | 5.12E−07 |
| 602 | A | 5.18E−07 |
| 603 | A | 1.11E−06 |
| 604 | A | 6.94E−07 |
| 605 | A | 9.06E−07 |
| 606 | A | 2.14E−06 |
| 608 | A | 7.92E−07 |
| 609 | A | 6.29E−07 |
| 610 | A | 4.23E−07 |
| 611 | A | 1.67E−06 |
| 612 | A | 3.15E−07 |
| 613 | A | 2.36E−06 |
| 619 | A | 1.36E−06 |
| 620 | A | 8.89E−07 |
| 621 | A | 2.52E−06 |
| 622 | A | 1.71E−05 |
| 623 | A | 3.00E−05 |
| 624 | A | 5.25E−07 |
| 625 | A | 6.59E−06 |
| 626 | A | 5.40E−07 |
| 627 | A | 1.33E−06 |
| 628 | A | 2.40E−06 |
| 629 | A | 8.80E−07 |
| 630 | A | 3.40E−07 |
| 631 | A | 3.17E−06 |
| 633 | A | 3.71E−07 |
| 634 | A | 4.23E−06 |
| 635 | A | 4.59E−06 |
| 636 | A | 3.08E−06 |
| 637 | A | 1.54E−06 |
| 638 | A | 3.73E−07 |
| 639 | A | 1.26E−05 |
| 640 | A | 1.81E−05 |
| 641 | B | 3.03E−06 |
| 642 | B | 4.60E−07 |
| 643 | A | 1.23E−06 |
| 645 | A | 6.17E−07 |
| 646 | A | 1.48E−07 |
| 647 | A | 8.71E−07 |
| 648 | A | 7.90E−07 |
| 650 | A | 9.12E−07 |
| 651 | A | 1.26E−06 |
| 652 | A | 4.71E−06 |
| 653 | B | 1.13E−07 |
| 654 | A | 7.65E−07 |
| 655 | A | 8.42E−06 |
| 656 | A | 2.91E−06 |
| 657 | A | 1.39E−07 |
| 658 | A | 8.79E−07 |
| 659 | A | 5.59E−07 |
| 660 | A | 6.20E−08 |
| 661 | A | 4.97E−06 |
| 662 | A | 9.06E−06 |
| 663 | A | 3.43E−06 |
| 664 | A | 3.03E−06 |
| 665 | A | 2.76E−07 |
| 666 | A | 1.07E−06 |
| 667 | A | 5.74E−07 |
| 668 | A | 3.55E−07 |
| 669 | A | 1.52E−06 |
| 670 | A | 3.60E−06 |
| 671 | A | 2.56E−06 |
| 672 | A | 3.91E−07 |
| 673 | A | 2.08E−07 |
| 674 | A | 1.71E−06 |
| 675 | A | 6.98E−06 |
| 676 | A | 6.56E−08 |
| 677 | A | 2.45E−07 |
| 678 | A | 9.63E−06 |
| 679 | A | 1.99E−07 |
| 680 | A | 6.87E−07 |
| 681 | A | 1.16E−06 |
| 682 | A | 5.18E−06 |
| 683 | A | 9.85E−06 |
| 684 | A | 5.85E−06 |
| 685 | A | 1.46E−06 |
| 686 | A | 4.42E−07 |
| 687 | A | 6.00E−07 |
| 688 | A | 2.34E−07 |
| 689 | A | 1.17E−07 |
| 690 | A | 1.41E−06 |
| 691 | A | 8.06E−07 |
| 692 | A | 1.32E−07 |
| 693 | A | 5.27E−07 |
| 694 | A | 6.29E−06 |
| 695 | A | 3.24E−06 |
| 696 | A | 5.07E−06 |
| 697 | A | 3.74E−07 |
| 698 | A | 5.43E−06 |
| 699 | A | 1.15E−07 |
| 700 | A | 3.30E−07 |
| 701 | A | 1.56E−06 |
| 702 | A | 4.15E−08 |
| 703 | A | 1.69E−07 |
| 704 | A | 2.40E−07 |
| 705 | A | 2.75E−06 |
| 706 | A | 9.39E−08 |
| 707 | B | 2.30E−07 |
| 708 | B | 1.07E−07 |
| 709 | A | 1.01E−06 |
| 710 | A | 3.28E−06 |
| 711 | A | 5.98E−08 |
| 712 | A | 7.40E−08 |
| 713 | A | 1.52E−07 |
| 714 | A | 1.26E−06 |
| 715 | B | 1.29E−06 |
| 716 | A | 3.11E−07 |
| 717 | B | 3.67E−07 |
| 718 | A | 1.59E−05 |
| 719 | A | 1.32E−05 |
| 721 | A | 6.70E−07 |
| 722 | A | 2.44E−07 |
| 729 | A | 8.64E−07 |
| 730 | A | 2.10E−07 |
| 733 | A | 2.11E−07 |
| 734 | A | 1.94E−07 |
| 735 | A | 4.33E−07 |
| 736 | A | 1.22E−06 |
| 737 | A | 1.91E−07 |
| 738 | A | 4.68E−07 |
| 739 | A | 8.72E−08 |
| 740 | A | 4.59E−08 |
| 741 | A | 1.32E−07 |
| 742 | A | 5.96E−08 |
| 743 | A | 1.10E−07 |
| 744 | A | 6.52E−08 |
| 745 | A | 6.64E−08 |
| 746 | A | 2.54E−08 |
| 747 | A | 1.18E−07 |
| 748 | A | 1.38E−07 |
| 749 | A | 7.24E−08 |
| 750 | B | 3.78E−07 |
| 751 | B | 2.79E−07 |
| 752 | B | 3.44E−07 |
| 753 | A | 1.11E−07 |
| 754 | A | 9.66E−08 |
| 755 | A | 5.84E−08 |
| 756 | A | 6.20E−08 |
| 757 | B | 2.00E−07 |
| 758 | B | 9.72E−08 |
| 759 | A | 1.85E−07 |
| 760 | A | 8.17E−08 |
| 762 | A | 3.90E−07 |
| 763 | B | 1.18E−07 |
| 765 | A | 2.10E−07 |
| 766 | A | 3.84E−07 |
| 767 | A | 7.88E−08 |
| 768 | B | 4.96E−08 |
| 769 | A | 1.25E−07 |
| 770 | A | 9.06E−08 |
| 771 | A | 1.83E−08 |
| 772 | A | 1.26E−07 |
| 773 | A | 3.22E−08 |
| 774 | A | 1.18E−07 |
| 775 | B | 8.44E−08 |
| 776 | A | 2.27E−07 |
| 777 | A | 1.75E−07 |
| 778 | B | 6.72E−08 |
| 779 | A | 1.43E−07 |
| 780 | A | 6.41E−07 |
| 781 | B | 8.23E−08 |
| 782 | B | 2.35E−07 |
| 783 | A | 4.30E−07 |
| 784 | B | 1.06E−07 |
| 785 | B | 6.10E−08 |
| 786 | B | 7.72E−08 |
| 787 | B | 1.03E−07 |
| 788 | B | 2.62E−07 |
| 789 | B | 2.70E−07 |
| 790 | B | 1.46E−06 |
| 791 | B | 1.92E−07 |
| 792 | B | 1.43E−07 |
| 793 | A | 8.57E−07 |
| 794 | B | 4.45E−08 |
| 797 | B | 2.51E−07 |
| 813 | B | 1.84E−07 |
| 814 | A | 1.35E−07 |
| 815 | B | 8.77E−08 |
| 816 | B | 1.01E−07 |
| 817 | B | 1.65E−07 |
| 818 | B | 1.06E−07 |
| 819 | B | 1.51E−07 |
| 821 | B | 5.36E−07 |
| 822 | B | 6.41E−08 |
| 823 | B | 1.28E−07 |
| 824 | B | 4.21E−07 |
| 835 | B | 1.46E−07 |
| 836 | B | 1.06E−07 |
| 837 | B | 3.10E−08 |
| 838 | B | 2.16E−08 |
| 840 | B | 3.05E−08 |
| 841 | A | 7.66E−08 |
| 842 | A | 8.10E−08 |
| 843 | B | 1.38E−06 |
| 844 | B | 2.92E−08 |
| 845 | B | 3.83E−08 |
| 846 | B | 4.20E−08 |
| 847 | B | 6.43E−08 |
| 848 | B | 4.86E−08 |
| 849 | B | 9.05E−08 |
| 850 | B | 5.09E−08 |
| 851 | B | 3.49E−08 |
| 852 | B | 2.35E−07 |
| 853 | B | 1.37E−07 |
| 854 | B | 1.47E−07 |
| 855 | A | 5.08E−08 |
| 856 | A | 7.48E−08 |
| 857 | A | 1.43E−07 |
| 858 | A | 5.06E−08 |
| 859 | B | 4.37E−08 |
| 860 | B | 9.30E−08 |
| 861 | A | 5.63E−08 |
| 862 | A | 7.38E−08 |
| 863 | A | 7.67E−08 |
| 864 | A | 1.83E−07 |
| 865 | A | 4.59E−08 |
| 867 | A | 7.30E−08 |
| 868 | A | 2.31E−07 |
| 869 | A | 1.93E−07 |
| 870 | B | 1.26E−07 |
| 871 | A | 1.14E−07 |
| 872 | B | 5.29E−08 |
| 873 | A | 3.41E−07 |
| 874 | B | 1.13E−07 |
| 875 | B | 5.97E−08 |
| 876 | B | 3.07E−07 |
| 877 | B | 5.05E−07 |
| 878 | B | 1.11E−06 |
| 879 | B | 1.36E−06 |
| 880 | B | 8.29E−07 |
| 881 | B | 1.53E−07 |
| 882 | B | 1.30E−06 |
| 883 | B | 1.33E−07 |
| 885 | B | 3.41E−07 |
| 886 | B | 3.81E−07 |
| 887 | B | 1.51E−07 |
| 888 | B | 9.25E−08 |
| 919 | B | 3.89E−08 |
| 920 | A | 1.04E−07 |
| 921 | B | 3.59E−08 |
| 930 | B | >3E−05 |
| 932 | B | 1.36E−05 |
| 933 | B | 1.89E−05 |
| 934 | B | >3E−05 |
| 935 | B | 1.90E−06 |
| 936 | B | 6.84E−06 |
| 938 | B | 4.42E−08 |
| 939 | B | 1.57E−07 |
| 940 | B | 2.66E−07 |
| 941 | B | 4.62E−08 |
| 942 | B | 2.22E−07 |
| 943 | B | 2.28E−07 |
| 944 | B | 2.13E−06 |
| 945 | B | 2.47E−05 |
| 946 | B | 3.42E−08 |
| 947 | B | 6.04E−07 |
| 948 | B | 7.12E−06 |
| 949 | B | >3E−05 |
| 950 | B | >3E−05 |
| 951 | B | 1.79E−06 |
| 952 | B | 9.60E−08 |
| 954 | B | 2.79E−06 |
| 955 | B | 1.13E−05 |
| 956 | B | 1.45E−05 |
| 957 | B | 4.70E−06 |
| 959 | B | 2.87E−08 |
| 960 | B | 7.75E−08 |
| 961 | B | 9.88E−08 |
| 962 | B | 4.28E−08 |
| 963 | B | 9.73E−08 |
| 964 | B | 1.09E−07 |
| 965 | B | 6.88E−08 |
| 966 | B | 4.83E−08 |
| 967 | B | 1.37E−07 |
| 968 | B | 7.27E−08 |
| 971 | B | 4.10E−08 |
| 972 | B | 3.10E−08 |
| 973 | B | 3.43E−08 |
| 974 | B | 6.04E−08 |
| 975 | B | 8.02E−08 |
| 976 | B | 7.25E−08 |
| 977 | B | 5.60E−08 |
| 978 | B | 1.64E−07 |
| 979 | B | 6.72E−08 |
| 980 | B | 1.42E−07 |
| 981 | B | 8.60E−08 |
| 982 | B | 1.57E−07 |
| 983 | B | 7.44E−08 |
| 984 | B | 3.55E−08 |
| 985 | B | 1.21E−07 |
| 986 | B | 8.69E−08 |
| 987 | B | 3.77E−08 |
| 988 | B | 2.04E−07 |
| 989 | B | 8.85E−08 |
| 990 | B | 3.53E−07 |
| 991 | B | 1.62E−07 |
| 992 | B | 1.84E−07 |
| 993 | B | 1.43E−07 |
| 994 | B | 5.97E−08 |
| 995 | B | 1.08E−07 |
| 996 | B | 8.45E−08 |
| 997 | B | 9.65E−07 |
| 998 | B | 6.31E−08 |

### Example C: Quantification of the Cleaved Form of PARP In Vivo

The ability of the compounds of the invention to induce apoptosis was evaluated in vivo using the AMO-1 multiple myeloma xenograft.

1×10⁷ cells were subcutaneously grafted into immunosuppressed SCID mice. 12-14 days later, when tumors had reached the appropriate volume, mice (3 per group) were treated once with the various compounds (per os, HEC 1%/Tween 80 99:1). Tumor samples were recovered 16 hours after dosing and lysed. Cleared lysates were used for cleaved PARP quantification (as a marker of apoptosis induction) using the Meso Scale Discovery (MSD) Elisa platform test. Results are expressed as the ratio between cleaved PARP in treated mice over control mice. As shown in Table below, compounds of the invention efficiently induced apoptosis in AMO-1 tumors in vivo:

| | | Cleaved PARP |
|---|---|---|
| Ex. | Dose (mpk) | (fold to control) |
| 42 | 120 | 5 |
| 44 | 30 | 0.7 |
| | 60 | 0.7 |
| | 120 | 1.4 |
| 708 | 120 | 12.6 |
| 768 | 30 | 0.8 |
| | 60 | 15.4 |
| | 120 | 30.3 |
| 781 | 30 | 11.6 |
| | 60 | 4.5 |
| 794 | 60 | 6.8 |
| | 120 | 12.1 |
| 815 | 60 | 2.3 |
| | 120 | 4.6 |
| 838 | 30 | 15 |
| | 60 | 7.8 |
| 840 | 30 | 19.2 |
| | 60 | 29.4 |
| 854 | 30 | 6.9 |
| | 60 | 26.4 |
| | 120 | 29.1 |
| 866 | 30 | 1.2 |
| | 60 | 6.5 |
| | 120 | 6.3 |
| 919 | 30 | 6.7 |
| | 60 | 9.7 |

### Example D: Anti-Tumor Activity In Vivo

The anti-tumor activity of the compounds of the invention was evaluated using AMO-1 multiple myeloma xenografts.

1×10⁷ cells were subcutaneously grafted into immunosuppressed SCID mice. 7-10 days later, when the tumor mass had reached an average volume of about 150 mm³, mice were treated with the various compounds once a week for 2 weeks (per os, HEC 1%/Tween 80 99:1). Tumor size was measured three times per week using electronic calipers.

At the end of the treatment cycle (d9), tumor growth inhibition (TGI) was calculated using the formula:

$$( 1 - \frac{Median ( DTV at Dx in treated group )}{Median ( DTV at Dx in control group )} ) \times 100 ,$$

wherein DTV (Delta Tumor Volume) at Dx was calculated as follows:

(Tumor Volume at Dx)−(Tumor Volume at Randomization).

As shown in Table below, compounds of the invention display significant anti-tumor activity during the treatment period (tumor growth inhibition (or TGI) ranging from 51.5% to 129.2% at the highest dose):

| Example | Dose (mpk) | % TGI (d9) |
|---|---|---|
| 41 | 30 | 1 |
| | 60 | 7.4 |
| | 120 | 66.8 |
| 42 | 60 | 25.8 |
| | 120 | 114.1 |
| 781 | 30 | 76.4 |
| | 60 | 107.8 |
| 784 | 30 | 103.6 |
| | 60 | 112 |
| 786 | 30 | 43.2 |
| | 60 | 98.9 |
| 794 | 60 | 70.2 |
| | 120 | 51.5 |
| 815 | 60 | 18.7 |
| | 120 | 66.8 |
| 838 | 60 | 129.2 |
| 840 | 30 | 83.8 |
| | 60 | 119.8 |
| 845 | 30 | 88.2 |
| | 60 | 128.3 |
| 854 | 30 | 32 |
| | 60 | 95.2 |
| | 120 | 111.8 |
| 919 | 60 | 121 |

### Example E: Quantification of the Cleaved Form of PARP In Vivo

The ability of the compounds of the invention to induce apoptosis was evaluated in vivo using the AMO-1 multiple myeloma xenograft.

1×10⁷ cells were subcutaneously grafted into immunosuppressed SCID mice. 12-14 days later, when tumors had reached the appropriate volume, mice (3 per group) were treated once with the various compounds (IV). Tumor samples were recovered 6 or 16 hours after dosing and lysed. Cleared lysates were used for cleaved PARP quantification (as a marker of apoptosis induction) using the Meso Scale Discovery (MSD) Elisa platform test. Results are expressed as the ratio between cleaved PARP in treated mice over control mice. As shown in the Table below, compounds of the invention efficiently induced apoptosis in AMO-1 tumors in vivo.

| | | | Cleaved PARP |
|---|---|---|---|
| Example | Dose (mpk) | Time-point (h) | (fold to control) |
| 784 | 15 | 6 | 227.5 |
| | 30 | 6 | 174.2 |
| | 30 | 16 | 129.9 |
| 840 | 15 | 6 | 127.8 |
| | 30 | 6 | 166.3 |
| | 30 | 16 | 138.2 |
| 845 | 15 | 6 | 188.7 |
| | 30 | 6 | 158.2 |
| | 30 | 16 | 116.4 |
| 847 | 7.5 | 6 | 185.9 |
| | 15 | 6 | 215 |
| 919 | 15 | 6 | 185.6 |
| | 30 | 6 | 199.9 |
| | 30 | 16 | 69.7 |

1-43. (canceled)

44. A compound of Formula (I): 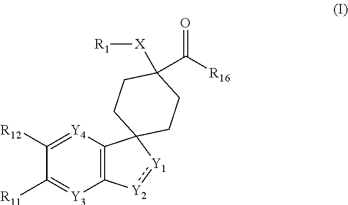
wherein:
X represents —S—, —O—, —CH₂— or —N(R₂)—,
Y₁ represents —C(R₄)(R₅)— or —N(R₆)—,
Y₂ represents —N(R₇)—, —C(R₈)(R₉)—, or —C(R₈)(R₉)—C(R₁₄)(R₁₅)—, [symbol]
represents a single bond or a double bond,
Y₃ represents —C(R₁₀)— or —N—,
Y₄ represents —C(R₁₃)— or —N—,
R₁ represents an aryl group or a heteroaryl group,
R₂ represents a hydrogen atom or a linear or branched (C₁-C₆)alkyl group,
or the pair (R₁,R₂) together with the nitrogen atom to which they are attached, forms a non-aromatic or aromatic mono- or bicyclic ring having from 5 to 12 ring members, which ring may have, in addition to the nitrogen, a second heteroatom selected from oxygen, sulphur and nitrogen, and which ring may be substituted by from 1 to 2 groups selected from a hydrogen atom, a halogen atom, and a linear or branched (C₁-C₆)alkyl group,
R₃ represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)(OR_{3A})₂, or —W₁—SO₂—OR_{3A}, —W₁—Cy₁, wherein:
W₁ represents a bond or a linear or branched (C₁-C₄)alkylene group,
R_{3A} and R_{3B}, independently of one another, represent a hydrogen atom, a linear or branched (C₁-C₆)alkyl group, a linear or branched (C₁-C₆)alkoxy(C₁-C₆)alkyl group, or a cycloalkyl group,
or the pair (R_{3A},R_{3B}) together with the nitrogen atom to which they are attached, forms a non-aromatic ring having from 4 to 7 ring members, which ring may have, in addition to the nitrogen, a second heteroatom selected from oxygen and nitrogen,
Cy₁ represents an aryl group or a heteroaryl group,
R₄ represents a hydrogen atom, a halogen atom, a linear or branched (C₁-C₆)alkyl group, a linear or branched (C₁-C₆)alkyl group substituted by 2 linear or branched (C₁-C₆)alkoxy groups, a linear or branched (C₂-C₆)alkenyl group, a linear or branched (C₂-C₆)alkynyl group, a linear or branched (C₁-C₆)alkoxy group, a linear or branched (C₂-C₆)alkenyloxy group, a linear or branched (C₁-C₆)alkoxy(C₁-C₆)alkyl group, a linear or branched (C₁-C₆)alkoxy(C₂-C₆)alkenyl group, a linear or branched (C₁-C₆)alkoxy(C₁-C₆)haloalkyl group, a hydroxy group, a linear or branched (C₁-C₆)hydroxyalkyl group, a —W₂-Cy₂ group, a —W₃-L-Cy₃ group, a —W₄—NR_{4A}R_{4B} group, or a —CO—NR_{4C}R_{4D} group, wherein:
W₂ represents a bond, a linear or branched (C₁-C₆)alkylene group, a linear or branched (C₂-C₆)alkenylene group, or a linear or branched (C₂-C₆)alkynylene group,
W₃ represents a bond, a linear or branched (C₁-C₆)alkylene group, a linear or branched (C₂-C₆)alkenylene group, a linear or branched (C₂-C₈)alkynylene group, a linear or branched (C₁-C₆)alkoxylene group, a linear or branched (C₁-C₄)hydroxyalkylene group, a linear or branched (C₁-C₄)haloalkylene group, or a —CH₂—CH(R_{4E})—CH₂— group,
W₄ represents a linear or branched (C₁-C₄)alkylene group,
L represents —O—, —S—, or —SO₂—,
R_{4A} and R_{4B}, independently of one another, represent a hydrogen atom, a linear or branched (C₁-C₆)alkyl group, an aryl group, a heteroaryl group, or an arylalkyl group,
R_{4C} and R_{4D}, independently of one another, represent a hydrogen atom, a linear or branched (C₁-C₆)alkyl group, an arylalkyl group, or a heteroarylalkyl group,
Cy₂ represents an aryl group, a heteroaryl group, a cycloalkyl group, a cycloalkenyl group, or a heterocycloalkyl group,
Cy₃ represents an aryl group, a heteroaryl group, a cycloalkyl group, a heterocycloalkyl group, an arylalkyl group, or a heteroarylalkyl group,
Cy₄ represents an aryl group, a heteroaryl group, an arylalkyl group, or a heterocycloalkylalkyl group,
R₅ represents a hydrogen atom or a linear or branched (C₁-C₆)alkyl group,
or the pair (R₄,R₅) represents an oxo group, or a cycloalkylidene group,
or the pair (R₄,R₅), together with carbon atoms to which they are attached, forms a non-aromatic ring having from 3 to 6 ring members,
R₆ represents an aryl group, a —SO₂-aryl group, or a —W₅—O—Cy₅ group, wherein:
W₅ represents a linear or branched (C₁-C₄)alkylene group,
Cy₅ represents an aryl group, or a heteroaryl group,
R₇ represents a hydrogen atom, a linear or branched (C₁-C₆)alkyl group, an arylalkyl group, or a formyl group,
R₈ represents a hydrogen atom, or a linear or branched (C₁-C₆)alkyl group,
or the pair (R₄,R₈), together with carbon atoms to which they are attached forms a non-aromatic or aromatic ring having from 3 to 7 ring members,
R₉ represents a hydrogen atom,
or the pair (R₈,R₉) represents an oxo group,
R₁₀ represents a hydrogen atom, a halogen atom, or a linear or branched (C₁-C₆)alkyl group,
or the pair (R₇,R₁₀) together with the nitrogen atom to which they are attached, forms a non-aromatic ring composed of from 4 to 7 ring members,
R₁₁ represents a hydrogen atom, a halogen atom, a linear or branched (C₁-C₆)alkyl group, or a linear or branched (C₁-C₆)alkoxy group,
R₁₂ represents a hydrogen atom, a halogen atom, a linear or branched (C₁-C₆)alkyl group, a linear or branched (C₁-C₆)alkenyl group, a linear or branched (C₁-C₆)alkynyl group, a linear or branched (C₁-C₆)alkoxy group, a linear or branched (C₁-C₆)alkenyloxy group, a linear or branched halo(C₁-C₆)alkyloxy group, a linear or branched (C₁-C₆)alkoxy(C₁-C₆)alkyl group, a linear or branched (C₁-C₆)alkoxy(C₁-C₆)alkoxy group, a hydroxy group, a linear or branched hydroxy(C₁-C₆)alkyl group, an acetyl group, a formyl group, a —CH₂—O-tetrahydrofuranyl group, —Cy₆, or —O—Cy₇, wherein:
Cy₆ represents an aryl group, a heteroaryl group, a cycloalkyl group, an arylalkyl group, or an arylalkenyl group,
Cy₇ represents an aryl group, cycloalkyl group, or a cycloalkylalkyl group,
or the pair (R₁₁,R₁₂), together with the carbon atoms to which they are attached, forms a non-aromatic ring having from 5 to 8 ring members, which ring may have 1 or 2 oxygen atoms, and which ring may be substituted by R₁₈ and R₁₈′,
R₁₃ represents a hydrogen atom, a halogen atom, or a linear or branched (C₁-C₆)alkyl group,
R₁₄ and R₁₅, independently of one another, represent a hydrogen atom or a linear or branched (C₁-C₆)alkyl group,
R₁₆ represents a —O—R₃ group or a —NR₁₇R₁₇′ group,
R₁₇ and R₁₇′ independently of one another, represent a hydrogen atom, a linear or branched (C₁-C₆)alkyl group, a —SO₂—CF₃ group, or a —SO₂—CH₃ group, and
R₁₈ and R₁₈′ independently of one another, represent a hydrogen atom, a halogen atom, or a linear or branched (C₁-C₆)alkyl group,
or the pair (R₁₈,R₁₈′), together with the carbon atoms to which they are attached, forms a non-aromatic ring having from 3 to 5 ring members,
wherein the aryl, heteroaryl, cycloalkyl, cycloalkenyl, heterocycloalkyl, arylalkyl, arylalkenyl, heteroarylalkyl, cycloalkylalkyl, and heterocycloalkylalkyl groups so defined may be optionally substituted by from 1 to 4 groups selected from halogen, linear or branched (C₁-C₆)alkyl, linear or branched halo(C₁-C₆)alkyl, linear or branched halo(C₁-C₆)alkylidene, linear or branched (C₁-C₆)alkoxy, linear or branched (C₁-C₆)alkoxy(C₁-C₆)alkoxy, a linear or branched halo(C₁-C₆)alkyloxy, hydroxy, linear or branched hydroxy(C₁-C₆)alkyl, cyano, oxo, —NR′R″, —C(O)—OR′, cyclopropyl, 2,2-dimethylcyclopropyl, phenyl, pyridinyl, benzyl, (2,3,6-trifluorophenyl)methyl, —CH₂-pyridinyl, —O-phenyl, —O-benzyl, —O-pyridinyl, —O—CH₂-cyclopropyl, —O—CH₂-pyridinyl, aryloxyalkyl, and heteroaryloxyalkyl, wherein R′ and R″ independently of one another, represent a hydrogen atom or linear or branched (C₁-C₆)alkyl,
its enantiomers, diastereoisomers, and addition salts thereof with a pharmaceutically acceptable acid or base.

45. The compound according to claim 44, wherein X represents —O— or —N(R₂)—.

46. The compound according to claim 44, wherein [symbol] represents a single bond.

47. The compound according to claim 44, wherein Y₁ represents —C(R₄)(R₅)—.

48. The compound according to claim 44, wherein Y₂ represents —C(R₈)(R₉)—.

49. The compound according to claim 44, which is a compound of Formula (I-e): 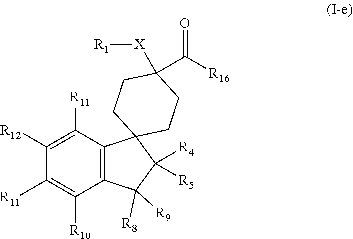
wherein R₁, R₄, R₅, R₈, R₉, R₁₀, R₁₁, R₁₂, R₁₃, R₁₆ and X are as defined in claim 44.

50. The compound according to claim 44, wherein R₁ represents an aryl group.

51. The compound according to claim 44, wherein R₁ represents an aryl group which is substituted by from 1 to 3 groups selected from halogen, linear or branched (C₁-C₆)alkyl, linear or branched halo(C₁-C₆)alkyl, linear or branched (C₁-C₆)alkoxy, cyano, and hydroxy.

52. The compound according to claim 44, wherein R₂ represents a hydrogen atom or a methyl group.

53. The compound according to claim 44, wherein R₁₆ represents a —O—R₃ group.

54. The compound according to claim 44, wherein R₃ represents a hydrogen atom, a linear or branched (C₁-C₆)alkyl group, a linear or branched halo(C₁-C₆)alkyl group, —W₁—OR_{3A}, —W₁—O—C(O)—R_{3A}, —W₁—NR_{3A}R_{3B}, —W₁—C(O)—NR_{3A}R_{3B}, —W₁—O—C(O)—OR_{3A}, —W₁—O—C(O)—NR_{3A}R_{3B}, —W₁—O—P(O)—(OR_{3A})₂, —W₁—SO₂—OR_{3A}, or —W₁-Cy₁, wherein:
W₁ represents a bond, a —CH₂— group, a —(CH₂)₂— group, a —(CH₂)₃— group, or a —CH(CH₃)— group,
R_{3A} and R_{3B}, independently of one another, represent a hydrogen atom, a methyl group, an ethyl group, an isopropyl group, a tert-butyl group, a methoxymethyl group, a methoxyethyl group, or a cyclopentyl group,
or the pair (R_{3A},R_{3B}), together with the nitrogen atom to which they are attached, forms a morpholinyl group, and
Cy₁ represents an indanyl group or a 5-methyl-2-oxo-1,3-dioxol-4-yl group.

55. The compound according to claim 44, wherein R₃ represents a hydrogen atom.

56. The compound according to claim 44, wherein R₄ represents a hydrogen atom, a halogen atom, a linear or branched (C₁-C₆)alkyl group, a branched (C₁-C₆)alkyl group substituted by 2 linear (C₁-C₆)alkoxy groups, a linear or branched (C₂-C₆)alkenyl group, a linear or branched (C₂-C₆)alkenyloxy group, a linear or branched (C₁-C₆)alkoxy(C₁-C₆)alkyl group, a linear or branched (C₁-C₆)alkoxy(C₂-C₆)alkenyl group, a linear or branched (C₁-C₆)alkoxy(C₁-C₆)haloalkyl group, a —W₂-Cy₂ group, a —W₃-L-Cy₃ group, a —W₄—NR_{4A}R_{4B} group, or a —CO—NR_{4C}R_{4D} group, wherein
W₂ represents a bond, a linear or branched (C₁-C₄)alkylene group, a linear or branched (C₂-C₄)alkenylene group, or a linear or branched (C₂-C₄)alkynylene group,
W₃ represents a bond, a linear or branched (C₁-C₆)alkylene group, a linear or branched (C₂-C₈)alkynylene group, a linear or branched (C₁-C₆)alkoxylene group, a branched (C₁-C₄)hydroxyalkylene group, a linear or branched (C₁-C₄)haloalkylene, or a —CH₂—CH(R_{4E})—CH₂— group,
L represents —O—, —S—, or —SO₂—,
Cy₂ represents an aryl group, a heteroaryl group, a cycloalkyl group, a cycloalkenyl group, or a heterocycloalkyl group, and
Cy₃ represents an aryl group, a heteroaryl group, a heterocycloalkyl group, an arylalkyl group, or a heteroarylalkyl group.

57. The compound according to claim 44, wherein R₄ represents a hydrogen atom, a bromine atom, a iodine atom, a methyl group, an ethyl group, a propyl group, a 3-methoxy-2-(methoxymethyl)propyl, a prop-1-en-1-yl group, a (prop-2-en-1-yl)oxy group, a methoxypropyl group, an ethoxypropryl group, a 3-ethoxyprop-1-en-1yl group, a 2,2-difluoro-3-methoxypropyl group, a —W₂-Cy₂ group, a —W₃-L-Cy₃ group, a —W₄—NR_{4A}R_{4B} group, or a —CO—NR_{4C}R_{4D} group.

58. The compound according to claim 44, wherein R₄ represents a —W₃-L-Cy₃ group.

59. The compound according to claim 44, wherein R₄ represents 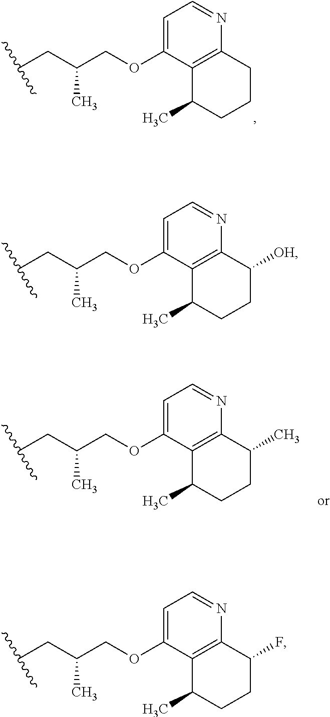 [SMILES: *C[C@@H](C)COc1ccnc2c1[C@H](C)CCC2 ; *C[C@@H](C)COc1ccnc2c1[C@H](C)CC[C@H]2O ; *C[C@@H](C)COc1ccnc2c1[C@H](C)CC[C@H]2C ; *C[C@@H](C)COc1ccnc2c1[C@H](C)CC[C@H]2F]
wherein the wavy line indicates the covalent attachment site to the spirocyclohexane scaffold.

60. The compound according to claim 44, wherein R₅ represents a hydrogen atom, or a methyl group.

61. The compound according to claim 44, wherein R₆ represents a phenyl group, an —SO₂-phenyl group, or a —W₅—O-Cy₅ group.

62. The compound according to claim 44, wherein R₇ represents a hydrogen atom, a methyl group, an ethyl group, a benzyl group, or a formyl group.

63. The compound according to claim 44, wherein R₈ represents a hydrogen atom, a methyl group, an ethyl group, or an isopropyl group.

64. The compound according to claim 44, wherein R₉ represents a hydrogen atom.

65. The compound according to claim 44, wherein R₁₀ represents a hydrogen atom, a chlorine atom, a fluorine atom, a bromine atom, or a methyl group.

66. The compound according to claim 44, wherein R₁₁ represents a hydrogen atom, a chlorine atom, a fluorine atom, a bromine atom, a methyl group, or a methoxy group.

67. The compound according to claim 44, wherein R₁₂ represents a hydrogen atom, a fluorine atom, a bromine atom, a iodine atom, a chlorine atom, a methyl group, an ethyl group, a prop-1-enyl group, a —C≡CH group, a methoxy group, an ethoxy group, a propoxy group, an isopropoxy group, an isobutyloxy group, a 2-methoxypropan-2-yl group, a prop-2-en-1-yloxy group, a 2,2,2-trifluoroethoxy group, a methoxymethyl group, a methoxyethoxy group, a methoxypropoxy group, a hydroxy group, a hydroxymethyl group, a 1-hydroxyethyl group, an acetyl group, a formyl group,
a —CH₂—O-tetrahydrofuranyl group, —Cy₆, or —O-Cy₇.

68. The compound according to claim 44, wherein the pair (R₁₁,R₁₂), together with the carbon atoms to which they are attached, forms a non-aromatic ring having from 5 to 8 ring members, which ring may have 1 or 2 oxygen atoms, and which ring may be substituted by R₁₈ and R_{˜}′.

69. The compound according to claim 44, wherein the pair (R₁₁R₁₂), together with the carbon atoms to which they are attached, forms a non-aromatic ring as follows: 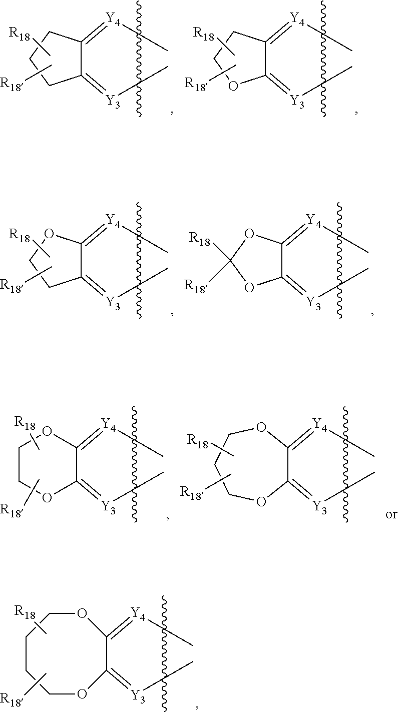
wherein R₁₈ and R₁₈′ are as defined in claim 44.

70. The compound according to claim 44, wherein R₁₃ represents a hydrogen atom, a fluorine atom, a bromine atom, or a methyl group.

71. The compound according to claim 44, which is selected from:
(1s,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid;
(1s,4S)-4-(3-chloro-4-fluoroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-isoindole]-4-carboxylic acid;
(1s,4S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid;
(1s,4S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-[1,3]dioxolo[4,5-f]isoindole]-4-carboxylic acid;
(1r,2′S,4S)-4-(3-chloro-2-methylanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-5′-fluoro-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-5′-fluoro-2′-[(2R)-3-{[(5R,8R)-8-hydroxy-5-methyl-5,6,7,8-tetrahydroquinolin-4-yl]oxy}-2-methylpropyl]-2′,3′-dihydrospiro[cyclohexane-1,1′-indene]-4-carboxylic acid;
(1r,2′S,4S)-5′-chloro-4-(3-chloroanilino)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-ethoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-2′-[(2R)-2-methyl-3-{[(5R)-5-methyl-5,6,7,8-tetrahydroquinolin-4-yl]oxy}propyl]-6′-[(propan-2-yl)oxy]-2′,3′-dihydrospiro[cyclohexane-1,1′-indene]-4-carboxylic acid;
(1r,2′S,4S)-4-(3-chloroanilino)-6′-(2-methoxyethoxy)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-5′-chloro-4-(3-chloroanilino)-6′-methoxy-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,4S,8'S)-4-(3-chloroanilino)-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid;
(1r,4S,7'S)-4-(3-chloroanilino)-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,4S,6'S)-4-(3-chloroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-4-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-2-fluoroanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloro-2-methylanilino)-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,4S,6'S)-4-(3-chloroanilino)-2′,2′-dimethyl-6′-[(2R)-2-methyl-3-{[(5R)-5-methyl-5,6,7,8-tetrahydroquinolin-4-yl]oxy}propyl]-6′,7′-dihydro-2′H-spiro[cyclohexane-1,5′-indeno[5,6-d][1,3]dioxole]-4-carboxylic acid;
(1r,2′S,4S)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′R,4R)-4-(3-chloroanilino)-5′,6′-dimethyl-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(1-hydroxyethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(2-methoxypropan-2-yl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,2′S,4S)-4-(3-chloroanilino)-6′-(methoxymethyl)-2′-[(2R)-2-methyl-3-{[(5R)-5-methyl-5,6,7,8-tetrahydroquinolin-4-yl]oxy}propyl]-2′,3′-dihydrospiro[cyclohexane-1,1′-indene]-4-carboxylic acid;
(1r,4S,6'S)-4-(3-chloroanilino)-6′-[(2R)-3-{[(5R,8R)-8-hydroxy-5-methyl-5,6,7,8-tetrahydroquinolin-4-yl]oxy}-2-methylpropyl]-6′,7′-dihydro-2′H-spiro[cyclohexane-1,5′-indeno[5,6-d][1,3]dioxole]-4-carboxylic acid;
(1r,4S,7'S)-4-(3-chloroanilino)-7′-[(2R)-3-{[(5R,8R)-8-hydroxy-5-methyl-5,6,7,8-tetrahydroquinolin-4-yl]oxy}-2-methylpropyl]-2′,3′,7′,8′-tetrahydrospiro[cyclohexane-1,6′-indeno[5,6-b][1,4]dioxine]-4-carboxylic acid;
(1r,3'S,4S,7'S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,3′R,4S,7'S)-4-(3-chloroanilino)-3′-methyl-7′-[(2R)-2-methyl-3-{[(5R)-5-methyl-5,6,7,8-tetrahydroquinolin-4-yl]oxy}propyl]-2′,3′,7′,8′-tetrahydrospiro[cyclohexane-1,6′-indeno[5,6-b][1,4]dioxine]-4-carboxylic acid;
(1r,4S,4'S,8'S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid; and
(1r,4S,4′R,8'S)-4-(3-chloroanilino)-4′-methyl-8′-[(2R)-2-methyl-3-{[(5R)-5-methyl-5,6,7,8-tetrahydroquinolin-4-yl]oxy}propyl]-3′,4′,8′,9′-tetrahydro-2′H-spiro[cyclohexane-1,7′-indeno[5,6-b][1,4]dioxepine]-4-carboxylic acid.

72. A process for preparing the compound of Formula (I) according to claim 44, employing a compound of Formula (IIIA): 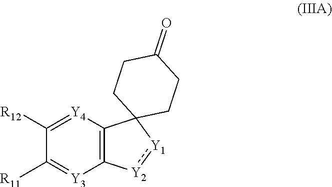
wherein R₁₁, R₁₂, Y₁, Y₂, Y₃, Y₄ and [symbol] are as defined in claim 44.

73. The process according to claim 72, wherein the compound of Formula (IIIA) is 6′-bromo-2′H-spiro[cyclohexane-1,5′-indeno[5,6-d][1,3]dioxol]-4-one.

74. A process for preparing the compound of Formula (I) according to claim 44, employing a compound of Formula (VA): 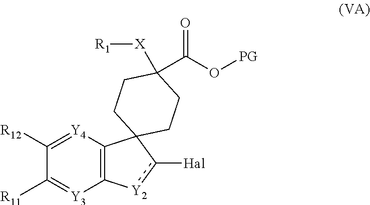
wherein R₁, R₁₁, R₁₂, X, Y₂, Y₃, Y₄ and [symbol] are as defined in claim 44, Hal represents a halogen atom, and PG represents a protecting group.

75. The process according to claim 74, wherein the compound of Formula (VA) is methyl (1s,4s)-2′-bromo-4-(3-chloroanilino)spiro[cyclohexane-1,1′-indene]-4-carboxylate or methyl (1s,4s)-6′-bromo-4-(3-chloroanilino)-2′H-spiro[cyclohexane-1,5′-indeno[5,6-d][1,3]dioxole]-4-carboxylate.

76. A pharmaceutical composition comprising the compound of Formula (I) according to claim 44, or an addition salt thereof with a pharmaceutically acceptable acid or base, in combination with one or more pharmaceutically acceptable excipients.

77. A method of treating a condition requiring an an anti-apoptotic inhibitor in a subject in need thereof, comprising administration of an effective amount of the compound according to claim 44, alone or in combination with one or more pharmaceutically acceptable excipients.

78. A method of treating a condition selected from cancer and auto-immune and immune system diseases in a subject in need thereof, comprising administration of an effective amount of the compound according to claim 44, alone or in combination with one or more pharmaceutically acceptable excipients.

79. The method according to claim 78, wherein the cancer is an haematological malignancy or a solid tumor.

80. The method according to claim 79, wherein the haematological malignancy is selected from myeloma, multiple myeloma, lymphoma, Non-Hodgkin Lymphoma (NHL), Diffuse Large B-cell Lymphoma (DLBCL), leukemia, Chronic Lymphocytic Leukemia (CLL), T-cell Acute Lymphoblastic Leukemia (r-ALL), B-cell Acute Lymphoblastic Leukemia (B-ALL) and Acute Myelogenous Leukemia (AML).

81. The method according to claim 79, wherein the solid tumor is selected from bladder, brain, breast, uterus, oesophagus and liver cancers, colorectal cancer, renal cancer, melanoma, ovarian cancer, prostate cancer, pancreatic cancer and lung cancer, including non-small-cell lung cancer and small-cell lung cancer.

82. A combination comprising the compound of Formula (I) according to claim 44 and an anti-cancer agent selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies.

83. A pharmaceutical composition comprising the combination according to claim 82 in combination with one or more pharmaceutically acceptable excipients.

84. A method of treating cancer in a subject in need thereof, comprising administration of an effective amount of the combination according to claim 82, alone or in combination with one or more pharmaceutically acceptable excipients.
